(19) United States
(12) Patent Application Publication
(10) Pub. No.: US 2026/0250290 A1
(43) Pub. Date: Aug. 27, 2026

(54) AMINO-SUBSTITUTED PYRROLOTRIAZINE DERIVATIVES AS INHIBITORS OF SGK1

(71) Applicant: BRISTOL-MYERS SQUIBB COMPANY, Princeton, NJ (US)

(72) Inventors: Peter W. Glunz, Yardley, PA (US); Zilun Hu, Jamison, PA (US); Daniel S. Gardner, Furlong, PA (US); Mimi L. Quan, Poway, CA (US); Vladimir Ladziata, Ewing, NJ (US); Shun Su, San Diego, CA (US); Adam James Clarke, Crosswicks, NJ (US); John S. Tokarski, Princeton, NJ (US); Andrea Bortolato, Metuchen, NJ (US); Susan Nicole Pieniazek, Robbinsville, NJ (US); John V. Duncia, Newtown, PA (US); Tarun Kumar Maishal, Bangalore (IN); Satheesh Kesavan Nair, Bangalore (IN); Nagalakshmi Pulicharla, Bangalore (IN); Sharanabasappa Patil, Raichur (IN); Kamalraj Thiyagarajan, Vellore (IN); Vadiraj Kurdekar, Hangal (IN); Soumyo Sen, Bangalore (IN); Pratik Pal, Bangalore (IN); Soodamani Thangavel, Krishnagir (IN)

(21) Appl. No.: 19/154,125

(22) PCT Filed: Feb. 13, 2024
(86) PCT No.: PCT/US2024/015503

§ 371 Date: Aug. 6, 2025

Related U.S. Application Data
(60) Provisional application No. 63/484,776, filed on Feb. 14, 2023.

(51) Int. Cl. C07D 487/04; A61K 31/53

(52) U.S. Cl. CPC C07D 487/04; A61K 31/53

(57) ABSTRACT

The present invention provides compounds of Formula (I) or stereoisomers, tautomers, or pharmaceutically acceptable salts thereof, wherein all the variables am as defined herein. These compounds are selective SGK1 inhibitors. This invention also relates to pharmaceutical compositions comprising these compounds and methods of treating disorders associated with serum- and glucocorticoid-regulated kinase 1 (SGK1) activity, such as cardiovascular disorders, fibrotic diseases, metabolic diseases, immune and inflammatory diseases, neurological disorders, and cancer, by using the compounds and pharmaceutical compositions.

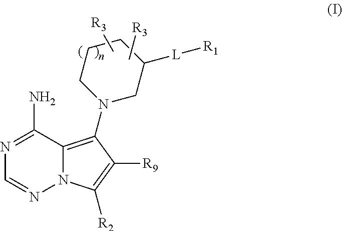

### CROSS REFERENCE TO RELATED APPLICATION

This application claims the benefit of U.S. Provisional Application No. 63/484,776, filed Feb. 14, 2023, the content of which is hereby incorporated herein by reference in its entirety.

### FIELD OF THE INVENTION

The present invention relates to novel amino-substituted pyrrolotriazine derivatives, compositions containing them, and methods of using them, for example, for the treatment or prophylaxis of disorders associated with aberrant serum and glucocorticoid-regulated kinase 1 (SGK1) activity.

### BACKGROUND OF THE INVENTION

Serum and glucocorticoid regulated kinase 1 (SGK1) belongs to a subfamily of serine/threonine kinase family consisting of SGK1, SGK2 and SGK3. Despite sharing 80% sequence homology at the catalytic domain and general properties of regulating ion channels, transport, and transcription for all three isoforms, SGK1 exhibits distinct patho-physiological functions (Lang, F. at al., Physiol Rev, 86(4), 1151-1178 (2006)).

SGK1 is expressed at low levels in most cells but is stimulated under a variety of pathological conditions such as excess glucocorticoid, mineralocorticoid, hypertension, inflammation, ischemia, and hyperglycemia (Lang, F. et al., Sci STKE, 108, re17 (2001)). Accumulating evidence suggests that over expression and excess activity of SGK1 may contribute to various diseases or disorders, such as cardiovascular and cerebrovascular diseases (including hypertension, heart failure, coronary artery disease, myocardial infarction, peripheral vascular disease, stroke and arrhythmia), fibrotic diseases (including diabetic nephropathy, glomerulonephritis, experimental nephrotic syndrome, obstructive nephropathy, lung fibrosis, liver cirrhosis, fibrotic pancreatitis, peritoneal fibrosis, Crohn's disease and coeliac disease), metabolic disorders and complications (including diabetes, obesity, metabolic syndrome), immune/inflammatory diseases (including rheumatoid arthritis and osteoarthritis), neuronal diseases (including Alzheimer, Parkinson. Seizure, and Lafora progressive myoclonic epilepsy), and cancer (Lang, F. et al., Expert Opin Investg Drugs. 22, 701-714 (2013)). Thus, targeting SGK1 may represent a therapeutic option for treating these disease conditions.

SGK1 activates tubular ion channels and transporters, and stimulates both Na⁺ intake and renal Na⁺ excretion. SGK1 is expected to influence blood pressure via regulation of salt balance. SGK1 is known to activate epithelial Na⁺ channel (ENAC), a channel regulated by mineralocorticoids. It is important to note that stimulation of Na⁺ reabsorption by mineralocorticoids is only partially mediated by SGK1. This is supported by the fact that gene deletion of SGK1 in mice had no effect on blood pressure under either normal conditions or after treatment with excess mineralocorticoids and excess salt (Wulff, P. et al., The Journal of Clinical Investigation, 110, 1263-1268 (2002)). However, under high-fructose or high-fat diet, hypertension induced by salt excess is attenuated in SGK1-deficient animals (Huang, D. Y. at al., Am J physiol Renal Physiol. 291, F1264-1273 (2006)). These data suggests that SGK1 also mediates insulin-dependent stimulation of renal Na⁺ retention and might contributes to the secondary hypertension in type II diabetes.

SGK1 is expressed in the heart and upregulated in heart failure (Das, S., et al., Circulation, 126, 2208-2219 (2012)). Substantial preclinical evidence suggests that SGK1, in response to aldosterone and other pathological stimuli, mediates multiple intracellular pathways which lead to cardiac hypertrophy, fibrosis and myocardial dysfunction. Aldosterone induces SGK1 gene expression in the heart, causing an increase of Na⁺ in the cytosol of cardiomyocytes and cardiac fibroblasts with the subsequent increase in Ca²⁺ through Na⁺/Ca²⁺ exchanger (Martin-Fernandez, B. et al., J Cardiovasc Pharmcol. 57, 114-121 (2011)).

In addition to mediating aldosterone effect, a broader role of SGK1 in fibrosis is suggested by the fact that SGK1 is upregulated by TGFβ and is involved in Smad2/3-TGFβ-CTGF signaling pathway, which leads to pathological fibrosis and negative remodeling (Artunc, F. et al., Nephron Physiol, 128, 35-39 (2014)). In several heart failure animal models, cardiac fibrosis and hypertrophy induced by deoxycorticosterone acetate (DOCA), angiotensin II or pressure overload are attenuated in SGK1 knockout mice (Das, S., et al., Circulation, 126, 2208-2219 (2012) and Vallon, V. et al., J Mol Med (Berl), 4, 396-404 (2006)). Furthermore, SGK1 is highly expressed in other fibrotic tissues and may play a role in diabetic nephropathy, glomerulonephritis, experimental nephrotic syndrome, obstructive nephropathy, lung fibrosis, liver cirrhosis, fibrotic pancreatitis, peritoneal fibrosis, Crohn's disease and coeliac disease (Lang, F. at al., Physiol Rev, 86(4), 1151-1178 (2006)). Therefore, inhibition of SGK1 kinase activity may provide benefit to other fibrotic diseases.

SGK1 may play a role in cardiac arrhythmia. In the heart, SGK1 stimulates the activity of several channels (SCN5A, Kv4.3, Kv1.5, and KCNE1/KCNQ1) affecting the shape and duration of the cardiac action potential. In fact, the E8CC/CT;I6CC gene variant of SGK1 is also reported to be associated with a shortened Q-T interval in humans (Busjahn, A. et al., Cell physiol Biochem, 14, 135-142 (2004)). Increased SGK1 expression and activity is also implicated in coagulation. SGK1 is stimulated by thrombin and oxidative stress, which in turn induces upregulation of tissue factor, a key trigger for extrinsic coagulation cascade (Belaiba, R S. et al., Circ Res. 98, 828-836 (2006)). An association between genetic variants in SGK1 and ischemic stroke has been reported (Dahlberg. J. et al., J Hypertens, 29, 884-889 (2011)).

SGK1 is expressed in glomerular podocytes and upregulated by aldosterone and oxidative stress. SGK1 phosphorylates nephrin and may participate in the development of proteinuria during mineralocorticoid excess, inflammation or other pathological stimuli (Ohashi, T. et al., Clin Exp Nephrol, 15, 688-693 (201)).

SGK1 stimulates the Na⁺-coupled glucose transporter SGLT1, which accelerates the intestinal uptake of glucose and contributes to the development of obesity. SGK1 is expressed in white adipose and is shown to stimulate adipocyte differentiation and adipogenesis (Lang, F. et al., Expert Opin Ther Targets. 13, 1303-1311 (2009)). Furthermore, a SGK1 polymorphism is reported to be associated with insulin secretion and development of type 2 diabetes mellitus (Schwab. M. et al., Cell Physiol Biochem. 21, 151-160 (2008)).

SGK1 appears to mediate the differentiation and maintenance of CD4⁺TH17 cells and is thus implicated in related disorders such as autoimmune and proinflammatory diseases (Kuchroo, V. K. et al., WO/2011014775). Furthermore, SGK1 is expressed in diseased osteoarthritic cartilage and is implicated in the pathogenesis of rheumatoid arthritis and osteoarthritis (Bartnik, E. et al., US 2008/0274487).

SGk1 is expressed in the brain and may contribute to the pathological effect of glucocorticoids during cerebral injury such as stroke and seizure (Kaufer, D. et al., Nat Neurosci, 7, 947-953 (2004)). In addition, SGK1 phosphorylates several neural factors involved in neurodegenerative diseases, although the role of SGK1 in the neuronal function is not well understood. For example, SGK1 phosphorylates tau, an activity that may be relevant to the development of Alzheimer's disease (Chuna, J. et al., Molecules & Cells (Springer Science & Business Media BV) 18 (3) (2004)). In addition, upregulation of SGK1 expression is shown to coincide with the onset of dopaminergic cell death in 1-year subacute MPTP model of Parkinson's disease (Stichel, C. C. et al., Eur J Neurosci. 21, 301-316 (2005)).

SGK1 is highly expressed in many tumor cells, these include but not limited to breast cancer, colon cancer, myeloma, medulloblastoma, non-small-cell lung cancer, ovarian cancer, and prostate cancer (Lang, F. at al., Physiol Rev, 86(4), 1151-1178 (2006)). As a down downstream regulator, SGK1 may mediate the effect of glucocorticoids on the invasiveness, motility and adhesiveness of breast cancer cells (Tangir, J. et al., Clin Exp Metastasis, 21, 477-483 (2004)). Furthermore, a potential role of SGK1 in immune-mediated tumor response is suggested (Heikamp, E. B. et al., Nat Immunol, 15, 457-464 (2014)).

Overall, SGK1 is a stress-responsive protein kinase that is upregulated by various pathological stimuli. Excess SGK1 activity is involved in cellular mechanisms that contribute to many disease states as described above. As such, inhibitors of SGK1, such as the compounds in the present invention, can be used to treat disease states in which SGK-1 activity contributes to the pathogenesis of these diseases.

### SUMMARY OF THE INVENTION

The present invention provides novel amino-substituted pyrrolotriazine derivatives including stereoisomers, tautomers, pharmaceutically acceptable salts, or solvates thereof, which are useful as selective inhibitors of serum- and glucocorticoid-regulated kinases 1 (SGK1).

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The present invention also provides methods of treating diseases, which methods comprise administering to a patient in need of such treatment compounds of the present invention as described above. Examples of such diseases that may be treated include, but are not limited to, cardiovascular and cerebrovascular diseases (including hypertension, heart failure, coronary artery disease, myocardial infarction, peripheral vascular disease, stroke and arrhythmia), fibrotic diseases (including diabetic nephropathy, glomerulonephritis, experimental nephrotic syndrome, obstructive nephropathy, lung fibrosis, liver cirrhosis, fibrotic pancreatitis, peritoneal fibrosis. Crohn's disease and coeliac disease), metabolic disorders and complications (including diabetes, obesity, metabolic syndrome), immune/inflammatory diseases (including rheumatoid arthritis and osteoarthritis), neuronal diseases (including Alzheimer, Parkinson, seizure, and Lafora progressive myoclonic epilepsy) and cancer.

The compounds of the invention may be used in the treatment and/or prophylaxis of conditions associated with aberrant SGK1 activity.

The compounds of the present invention may be used in therapy.

The compounds of the present invention may be used for the manufacture of a medicament for the treatment and/or prophylaxis of a condition associated with aberrant SGK1 activity.

The compounds of the invention can be used alone, in combination with other compounds of the present invention, or in combination with one or more, preferably one to two other agent(s).

These and other features of the invention will be set forth in expanded form as the disclosure continues.

### DETAILED DESCRIPTION OF THE INVENTION

The present application provides compounds, including all stereoisomers, solvates, prodrugs and pharmaceutically acceptable salts and solvate forms thereof, according to Formula (I). The present application also provides pharmaceutical compositions containing at least one compound according to Formula (I), or a stereoisomer, a tautomer, or a pharmaceutically acceptable salt or a solvate thereof, and optionally at least one additional therapeutic agent. Additionally, the present application provides methods for treating a patient suffering from an SGK1-modulated diseases or disorders, for example, cardiovascular and cerebrovascular diseases (including hypertension, heart failure, coronary artery disease, myocardial infarction, peripheral vascular disease, stroke and arrhythmia), fibrotic diseases (including diabetic nephropathy, glomerulonephritis, experimental nephrotic syndrome, obstructive nephropathy, lung fibrosis, liver cirrhosis, fibrotic pancreatitis, peritoneal fibrosis, Crohn's disease and coeliac disease), metabolic disorders and complications (including diabetes, obesity, metabolic syndrome), immune/inflammatory diseases (including rheumatoid arthritis and osteoarthritis), neuronal diseases (including Alzheimer, Parkinson, Seizure, and Lafora progressive myoclonic epilepsy) and cancer, by administering to a patient in need of such treatment a therapeutically effective amount of a compound of the present invention, or a stereoisomer, a tautomer, or a pharmaceutically acceptable salt or a solvate thereof, and optionally in combination with at least one additional therapeutic agent.

### I. Compounds of the Invention

In a first aspect, the present invention provides, inter alia, compounds of Formula (I):

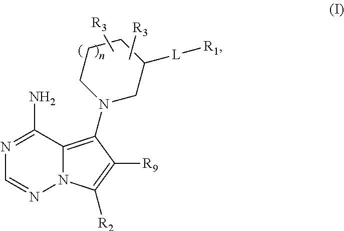

or stereoisomers, tautomers, pharmaceutically acceptable salts or solvates thereof,
wherein:
L is selected from the group consisting of —C(═O)NR₈—, —C(═O)O—, —NR₈C(═O)—, —NR₈C(═O)NR₈—, —NR₈C(═O)O—, and —NR₈S(═O)ₚ—;
R₁ is selected from the group consisting of C₁₋₅ alkyl, —(CR_{d}R_{d})ᵣ—C₃₋₁₀carbocyclyl substituted with 1-5 R₄, and 4- to 10-membered heterocyclyl comprising carbon atoms and 1-5 heteroatoms selected from N, NR₄ₐ, O, and S(═O)ₚ, and substituted with 1-5 R₄;
R₂ is selected from the group consisting of CN, CONHR₉, C₃₋₁₀ carbocyclyl substituted with 1-5 R₆, 3- to 10-membered heterocyclyl comprising carbon atoms and 1-5 heteroatoms selected from N, NR₆ₐ, O, and S(═O)ₚ, and substituted with 1-5 R₆, and C₂₋₃ alkynyl substituted with 0-1 C₃₋₁₀carbocyclyl substituted with 1-5 R₆ or 3- to 10-membered heterocyclyl comprising carbon atoms and 1-5 heteroatoms selected from N, NR₆ₐ, O, and S(═O)ₚ, and substituted with 1-5 R₆;
R₃ is selected from the group consisting of H, halo, —OR_{b}, —NRₐRₐ and C₁₋₃ alkyl substituted with 0-4 halo, OR_{b}, or NRₐRₐ substituents; alternatively, two adjacent R₃ groups are taken together with the carbon atoms to which they are attached to form a C₃₋₆ cycloalkyl;
R₄, at each occurrence, is independently selected from the group consisting of H, F, Cl, Br, ═O, CN, NO₂, —OR_{b}, —(CR_{d}R_{d})ᵣNRₐRₐ, —O(CR_{d}R_{d})₁₋₅OR_{b}, —O(CR_{d}R_{d})ᵣC(═O)NRₐRₐ, —O(CR_{d}R_{d})₁₋₅NRₐC(═O)R_{b}, —O(CR_{d}R_{d})₁₋₅NRₐC(═O)OR_{b}, —O(CR_{d}R_{d})₁₋₅NRₐRₐ, —C(═O)NRₐRₐ, —C(═O)R_{b}, —NRₐC(═O)OR_{b}, —NRₐC(═O)(CR_{d}R_{d})ᵣNRₐRₐ, S(═O)R₄, S(═O)NRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₂ carbocyclyl substituted with 1-5 R₅, —(CR_{d}R_{d})ᵣ—O—(CR_{d}R_{d})ᵣ—C₃₋₁₂ carbocyclyl substituted with 1-5 R₅, —(CR_{d}R_{d})ᵣ-3- to 18-membered heterocyclyl comprising carbon atoms and 1-4 heteroatoms selected from N, NR₅ₐ, O, and S(═O)ₚ, and substituted with 1-5 R₅; and —(CR_{d}R_{d})ᵣ—O—(CR_{d}R_{d})ᵣ-3- to 18-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₁₀carbocyclyl substituted with 1-5 R₅, and —(CR_{d}R_{d})ᵣ-3- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CR_{d}R_{d})ᵣ—NR₁₀R₁₀, C₁₋₆ alkyl substituted with 1-5 Rₑ, C₂₋₄ alkenyl substituted with 1-5 Rₑ, C₂₋₆ alkynyl substituted with 1-5 Rₑ, C₃₋₁₀ carbocyclyl with 1-5 Rₑ, and 3- to 10-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₆ alkyl substituted with 1-5 Rₑ, C(═O)R_{b}, C(═O)OR_{b}, C(═O)NRₐRₐ, S(═O)ₚR_{c}, S(═O)ₚNRₐRₐ, C₃₋₁₀ carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₆ alkyl substituted with 1-5 Rₑ;
R₆, at each occurrence, is independently selected from the group consisting of H, F, Cl, Br, CN, ═O, —(CR_{d}R_{d})ᵣNRₐRₐ, NO₂, —OR_{b}, —C(═O)NRₐRₐ, —C(═O)NRₐ(CR_{d}R_{d})ᵣOR_{b}, —C(═O)R_{b}, —C(═O)(CR_{d}R_{d})OR_{b}, —NRₐC(═O)OR_{b}, —NRₐC(═O)(CR_{d}R_{d})ᵣNRₐRₐ, —S(═O)_{P}R_{c}, —NRₐS(═O)_{P}R_{c}, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣ—C₃₋₁₀ carbocyclyl substituted with 1-5 R₇, and —(CR_{d}R_{d})ᵣ-3- to 10-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —(CR_{d}R_{d})ᵣC(═O)NRₐRₐ, —(CR_{d}R_{d}C)ᵣ(═O)R_{b}, —(CR_{d}R_{d})ᵣC(═O)(CR_{d}R_{d})ᵣOR_{b}, —(CR_{d}R_{d})ᵣC(═O)(CR_{d}R_{d})ᵣNRₐC(═O)R_{b}, —(CR_{d}R_{d})ᵣS(═O)ₚR_{c}, —(CR_{d}R_{d})ᵣS(═O)ₚNRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₁₀carbocyclyl substituted with 1-5 R₇, and —(CR_{d}R_{d})ᵣ-3- to 10-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₆ alkyl substituted with 1-5 Rₑ, C₂₋₆ alkenyl substituted with 1-5 Rₑ, and C₂₋₆ alkynyl substituted with 1-5 Rₑ;
R₇ₐ, at each occurrence, is independently selected from the group consisting of H and C₁₋₆ alkyl substituted with 1-5 Rₑ;
R₈, at each occurrence, is independently selected from the group consisting of H and C₁₋₃ alkyl substituted with 0-4 halo, OR_{b}, or NRₐRₐ;
R₉ is selected from the group consisting of H and C₁₋₃ alkyl;
R₁₀, at each occurrence, is independently selected from the group consisting of H, C₁₋₇ alkyl substituted with 1-5 Rₑ, —(CR_{d}R_{d})ᵣ—C₃₋₁₀carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
Rₐ, at each occurrence, is independently selected from the group consisting of H, —C(═O)OR_{b}, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣ—C₃₋₁₀carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or Rₐ and Rₐ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
R_{b}, at each occurrence, is independently selected from the group consisting of H, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣ—C₃₋₁₀carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ;
R_{c}, at each occurrence, is independently selected from the group consisting of F, Cl, C₁₋₆ alkyl substituted with 1-5 Rₑ, C₂₋₆alkenyl substituted with 1-5 Rₑ, C₂₋₆alkynyl substituted with 1-5 Rₑ, C₃₋₁₀carbocyclyl substituted with 1-5 Rₑ, and 3- to 10-membered heterocyclyl comprising carbon atoms and 1-4 heteroatoms selected from the group consisting of N, NR_{f}, O, and S(═O)ₚ, and substituted with 1-5 Rₑ;
R_{d}, at each occurrence, is independently selected from the group consisting of H, C₁₋₆ alkyl substituted with 1-5 Rₑ, and C₃₋₆ cycloalkyl substituted with 1-5 Rₜ;
Rₑ is independently selected from the group consisting of H, F, Cl, Br, CN, ═O, C₁₋₆alkyl substituted with 1-5 R_{g}, C₂₋₆alkenyl substituted with 1-5 R_{g}, C₂₋₆alkynyl substituted with 1-5 R_{g}, —(CH₂)ᵣ—C₃₋₁₀ carbocyclyl substituted with 1-5 R_{g}, —(CH₂)ᵣ-3- to 10-membered heterocyclyl comprising carbon atoms and 1-4 heteroatoms selected from the group consisting of N, NR_{f}, O, and S(═O)ₚ, and substituted with 1-5 R_{g}, —(CH₂)ᵣOR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₆alkyl substituted with 1-3 R_{g}, C₃₋₁₀ carbocyclyl substituted with 1-3 R_{g}, a 3- to 10-membered heterocyclyl comprising carbon atoms and 1-4 heteroatoms selected from the group consisting of N, NC₁₋₄ alkyl, O, and S(═O)ₚ, and substituted with 1-3 R_{g}; or R_{f} and R_{f} together with the nitrogen atom to which they are both attached form a 3- to 10-membered heterocyclyl comprising carbon atoms and 1-4 heteroatoms selected from the group consisting of N, NC₁₋₄ alkyl, O, and S(═O)ₚ, and substituted with 1-3 R_{g};
R_{g}, at each occurrence, is independently selected from the group consisting of H, F, Cl, Br, CN, —OH, —O(C₁₋₅ alkyl), NH₂, NH(C₁₋₅ alkyl), NH(C₁₋₅ alkyl)₂, C₁₋₅ alkyl, C₃₋₁₀ carbocyclyl, and a 3- to 10-membered heterocyclyl comprising carbon atoms and 1-4 heteroatoms selected from the group consisting of N, NC₁₋₄ alkyl, O, and S(═O)ₚ;
n is an integer of zero, 1, or 2;
p is an integer of zero, 1, or 2; and
r is an integer of zero, 1, 2, 3, 4, or 5.

In a second aspect within the scope of the first aspect, the present invention provides compounds of Formula (II):

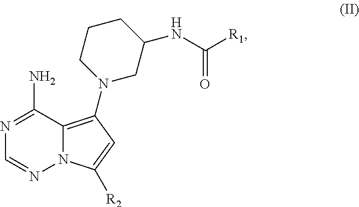

or stereoisomers, tautomers, pharmaceutically acceptable salts or solvates thereof,
wherein:
R₁ is selected from the group consisting of

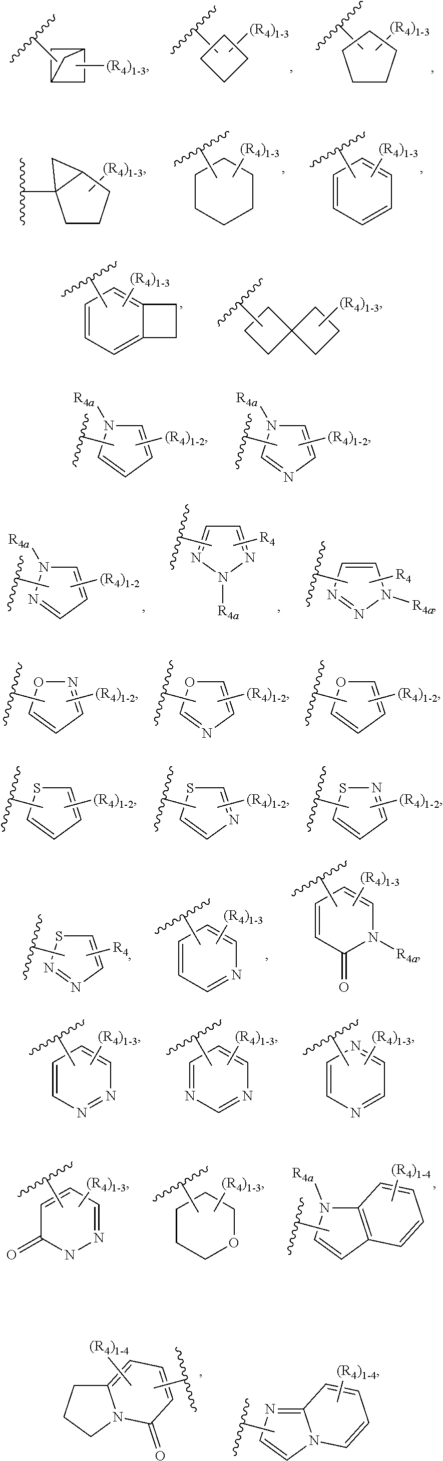

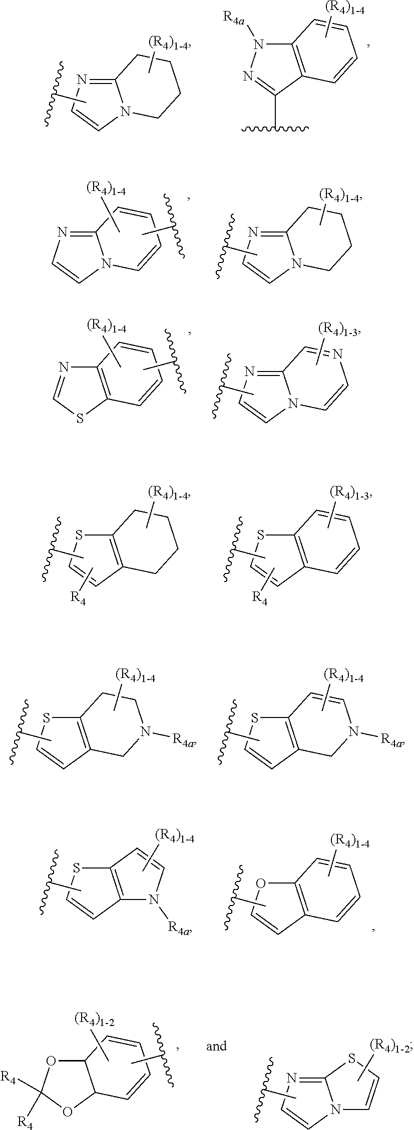

R₂ is selected from the group consisting of

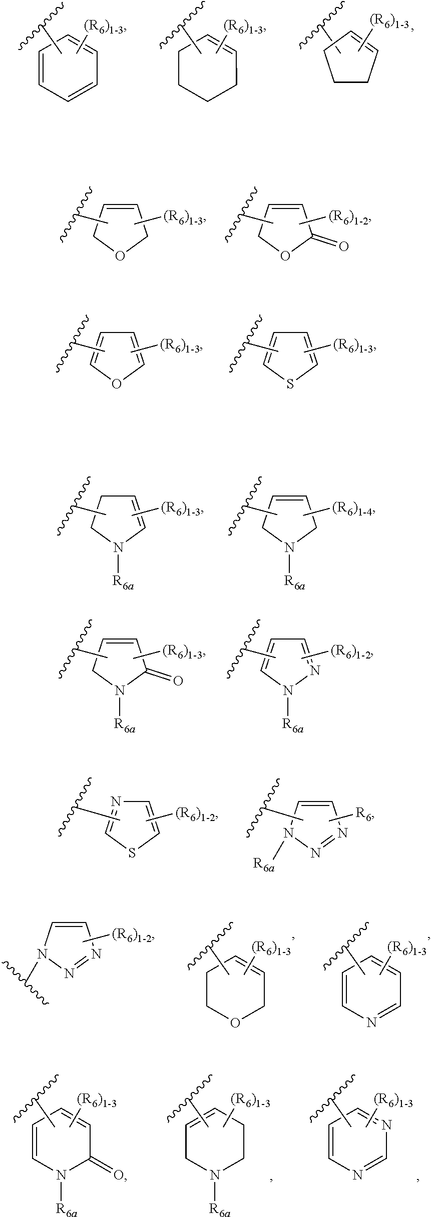

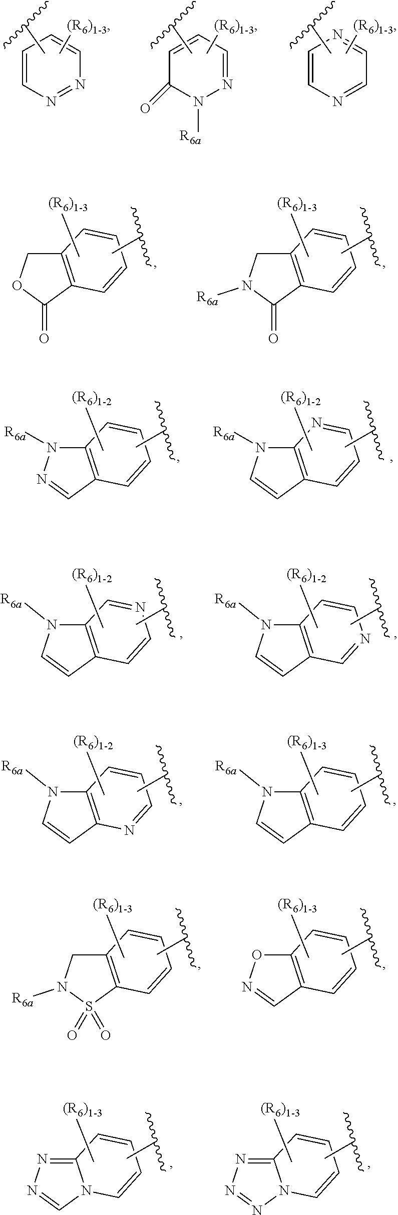

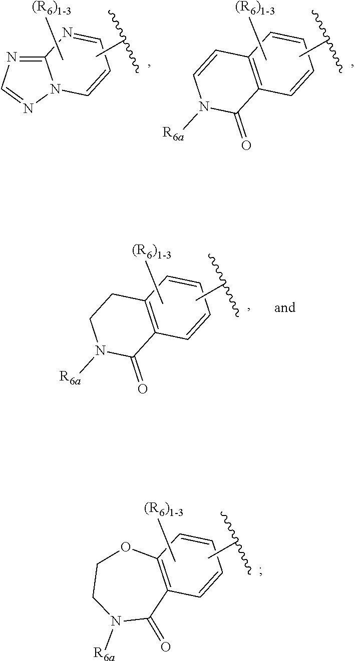

R₄, at each occurrence, is independently selected from the group consisting of H, F, Cl, Br, ═O, CN, —(CH₂)ᵣNRₐRₐ, —OR_{b}, —O(CH₂)₁₋₄OR_{b}, —O(CH₂)ᵣC(═O)NRₐRₐ, —O(CH₂)₁₋₄NRₐC(═O)R_{b}, —O(CH₂)₁₋₄NRₐC(═O)OR_{b}, —O(CH₂)₁₋₄NRₐRₐ, —C(═O)NRₐRₐ, —C(═O)R_{b}, —NRₐC(═O)OR_{b}, —NRₐC(═O)(CH₂)ᵣNRₐRₐ, C₁₋₅ alkyl substituted with 1-5 Rₑ, C₂₋₅ alkenyl substituted with 1-5 Rₑ, C₂₋₅ alkynyl substituted with 1-5 Rₑ; —(CH₂)ᵣ—C₃₋₁₀ carbocyclyl substituted with 1-5 R₅, —(CH₂)ᵣ—O—(CH₂)ᵣ—C₃₋₁₀carbocyclyl substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, C₁₋₅ alkyl substituted with 1-5 Rₑ, C₂₋₅ alkenyl substituted with 1-5 Rₑ, —(CH₂)ᵣ—C₃₋₆ carbocyclyl substituted with 1-5 R₅, and —(CH₂)ᵣ-5- to 9-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, —OR_{b}, ═O, —(CH₂)ᵣ—NR₁₀R₁₀, C₁₋₅ alkyl substituted with 1-5 Rₑ, C₂₋₅ alkenyl substituted with 1-5 Rₑ, C₂₋₅ alkynyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₅ alkyl substituted with 1-5 Rₑ, C(═O)R_{b}, C(═O)OR_{b}, C(═O)NRₐRₐ, S(═O)ₚR_{c}, S(═O)ₚNRₐRₐ, C₃₋₆carbocyclyl substituted with 1-5 Rₑ, and 5- to 9-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₅ alkyl substituted with 1-5 Rₑ;
R₆, at each occurrence, is independently selected from the group consisting of H, F, Cl, Br, CN, ═O, —(CH₂)ᵣNRₐRₐ, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)_{P}R_{c}, —NRₐS(═O)_{P}R_{c}, C₁₋₅ alkyl substituted with 1-5 Rₑ, C₂₋₅ alkenyl substituted with 1-5 Rₑ, —(CH₂)ᵣ—C₃₋₆carbocyclyl substituted with 1-5 R₇, and —(CH₂)ᵣ-5- to 9-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —(CH₂)ᵣC(═O)NRₐRₐ, —(CH₂)ᵣC(═O)R_{b}, (CH₂)ᵣC(═O)(CHR_{d})ᵣOR_{b}, —(CH₂)ᵣS(═O)ₚRₑ, —(CH₂)ᵣS(═O)ₚNRₐRₐ, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)ᵣ—C₃₋₆ carbocyclyl substituted with 1-5 R₇, and —(CH₂)ᵣ-4- to 9-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₅ alkyl substituted with 1-5 Rₑ, C₂₋₅ alkenyl substituted with 1-5 Rₑ, and C₂₋₅ alkynyl substituted with 1-5 Rₑ;
R₇ₐ, at each occurrence, is independently selected from the group consisting of H and C₁₋₅ alkyl substituted with 1-5 Rₑ;
R₁₀, at each occurrence, is independently selected from the group consisting of H, C₁₋₆ alkyl substituted with 1-5 Rₑ, —(CH₂)ᵣ—C₃₋₁₀carbocyclyl substituted with 1-5 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
Rₐ is independently selected from the group consisting of H, C(═O)OR_{b}, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)ᵣ—C₃₋₁₀carbocyclyl substituted with 1-5 Rₑ, and —(CH₂)ᵣ-3- to 9-membered heterocyclyl comprising carbon atoms and 1-4 heteroatoms selected from the group consisting of N, NR_{f}, O, and S(═O)ₚ, and substituted with 1-5 Rₑ; or Rₐ and Rₐ together with the nitrogen atom to which they are both attached form a 4- to 9-membered heterocyclyl comprising carbon atoms and 1-4 heteroatoms selected from the group consisting of N, NR_{f}, O, and S(═O)ₚ, and substituted with 1-5 Rₑ;
R_{b}, at each occurrence, is independently selected from the group consisting of H, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)ᵣ—C₃₋₁₀carbocyclyl substituted with 1-5 Rₑ, and —(CH₂)ᵣ-4- to 9-membered heterocyclyl comprising carbon atoms and 1-4 heteroatoms selected from the group consisting of N, NR_{f}, O, and S(═O)ₚ, and substituted with 1-5 Rₑ;
R_{c}, at each occurrence, is independently selected from the group consisting of C₁₋₅ alkyl substituted with 1-5 Rₑ, C₂₋₅ alkenyl substituted with 1-5 Rₑ, C₂₋₅ alkynyl substituted with 1-5 Rₑ, C₃₋₆carbocyclyl substituted with 1-5 Rₑ, and 4- to 9-membered heterocyclyl comprising carbon atoms and 1-4 heteroatoms selected from the group consisting of N, NR_{f}, O, and S(═O)ₚ, and substituted with 1-5 Rₑ;
R_{d}, at each occurrence, is independently selected from the group consisting of H, C₁₋₃ alkyl substituted with 1-4 Rₑ, and C₃₋₆ cycloalkyl substituted with 1-4 Rₑ;
Rₑ, at each occurrence, is independently selected from the group consisting of H, F, Cl, Br, CN, ═O, C₁₋₅ alkyl substituted with 1-4 R_{g}, C₂₋₃ alkenyl substituted with 1-4 R_{g}, C₂₋₅ alkynyl substituted with 1-4 R_{g}, —(CH₂)ᵣ—C₃₋₆ cycloalkyl substituted with 1-4 R_{g}, —(CH₂)ᵣ-4- to 9-membered heterocyclyl comprising carbon atoms and 1-4 heteroatoms selected from the group consisting of N, NR_{f}, O, and S(═O)ₚ, and substituted with 1-4 R_{g}, —(CH₂)ᵣOR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a 4- to 9-membered heterocyclyl;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4 or 5.

In a third aspect within the scope of the second aspect, the present invention provides compounds of Formula (II), or stereoisomers, tautomers, pharmaceutically acceptable salts or solvates thereof, wherein:
R₁ is selected from the group consisting of

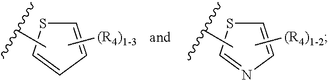

R₂ is selected from the group consisting of

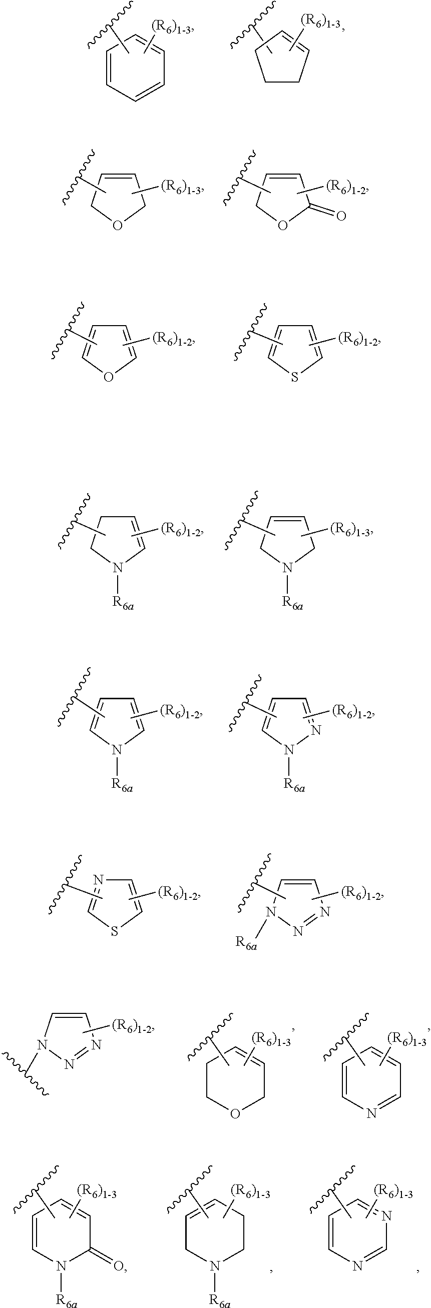

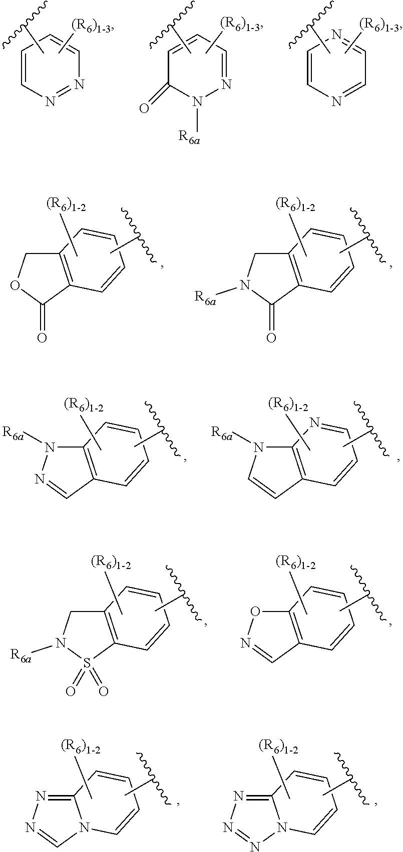

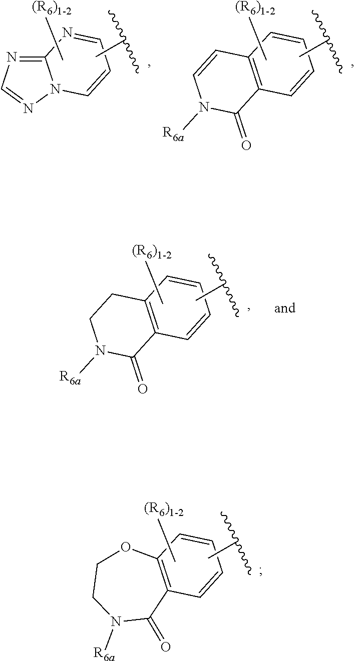

R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₁₀carbocyclyl substituted with 1-5 R₅, —(CH₂)ᵣ—O—(CH₂)ᵣ—C₃₋₁₀carbocyclyl substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CH₂)ᵣ—NR₁₀R₁₀, C₁₋₅ alkyl substituted with 1-4 Rₑ, C₃₋₆ carbocyclyl with 1-4 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₅ alkyl substituted with 1-4 Rₑ, C(═O)R_{b}, C(═O)OR_{b}, C₃₋₆ carbocyclyl substituted with 1-4 Rₑ, and 5- to 9-membered heterocyclyl comprising carbon atoms and 1-4 heteroatoms selected from the group consisting of N, NR_{5b}, O, and S(═O)ₚ, and substituted with 1-4 Rₑ;
R_{5b}, at each occurrence, is independently selected from the group consisting of H and C₁₋₄ alkyl substituted with 1-4 Rₑ;
R₆, at each occurrence, is independently selected from the group consisting of H, F, Cl, Br, CN, ═O, —(CH₂)ᵣNRₐRₐ, NO₂, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂R_{c}, —NRₐS(═O)₂R_{c}, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₄ alkynyl substituted with 1-5 Rₑ, —(CH₂)ᵣC₃₋₆carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ, at each occurrence, is independently selected from the group consisting of H and C₁₋₄ alkyl substituted with 1-5 Rₑ;
R₁₀, at each occurrence, is independently selected from the group consisting of H, C₁₋₅ alkyl substituted with 1-4 Rₑ, —(CH₂)ᵣ—C₃₋₁₀carbocyclyl substituted with 1-4 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-4 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-4 Rₑ;
Rₐ, at each occurrence, is independently selected from the group consisting of H, C₁₋₅ alkyl substituted with 1-4 Rₑ, C₃₋₁₀carbocyclyl substituted with 1-4 Rₑ, and —(CH₂)ᵣ-4- to 9-membered heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a substituted with 1-5 R₅ heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 1-4 Rₑ, C₂₋₅ alkynyl substituted with 1-4 Rₑ, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of C₁₋₅ alkyl substituted with 1-4 Rₑ, C₃₋₆carbocyclyl substituted with 1-4 Rₑ, and heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H, C₁₋₄ alkyl, and C₃₋₆ cycloalkyl;
Rₑ, at each occurrence, is independently selected from the group consisting of H, F, Cl, Br, CN, ═O, C₁₋₅ alkyl substituted with 1-4 R_{g}, C₂₋₅ alkenyl substituted with 1-4 R_{g}, C₂₋₅ alkynyl substituted with 1-4 R_{g}, —(CH₂)ᵣ—C₃₋₆ cycloalkyl, —(CH₂)ᵣ-4- to 8-membered heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a 4- to 8-membered heterocyclic ring;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4, or 5.

In a fourth aspect within the scope of the third aspect, the present invention provides compounds of Formula (II), or stereoisomers, tautomers, pharmaceutically acceptable salts or solvates thereof, wherein:
R₁ is selected from the group consisting of

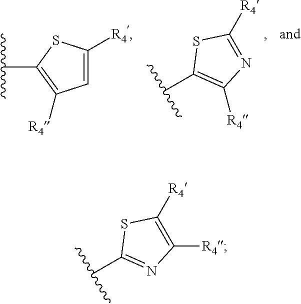

R₂ is selected from the group consisting

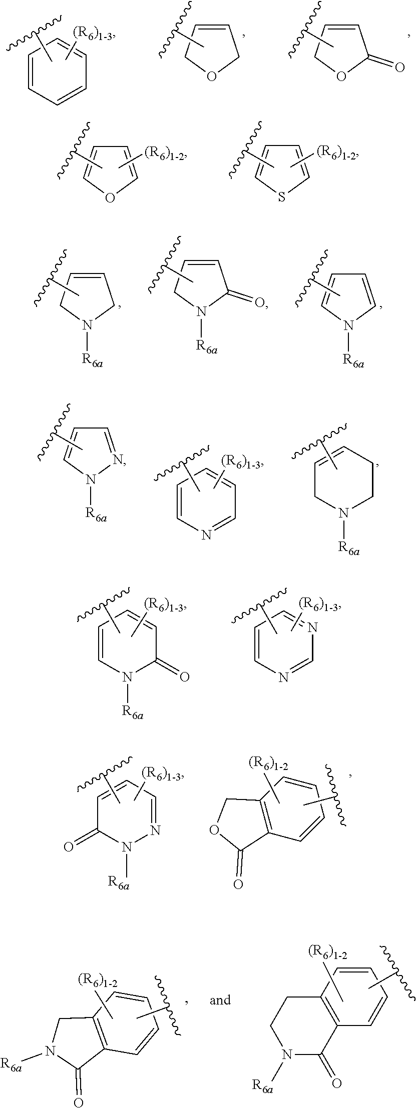

R₄′ is selected from the group consisting of H, F, Cl, CN, and C₁₋₄ alkyl substituted with 1-3 Rₑ;
R₄″ is selected from the group consisting of H, F, —O(CH₂)₁₋₃OR_{b}, —O(CH₂)₁₋₃C(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, C₃₋₁₀ carbocyclyl substituted with 1-5 R₅, —(CH₂)₀₋₄—O—(CH₂)₀₋₄ C₃₋₁₀ carbocyclyl substituted with 1-5 R₅, —(CH₂)₀₋₄-4- to 10-membered heterocyclyl comprising carbon atoms and 1-4 heteroatoms selected from N, NR₅ₐ, O, and S(═O)ₚ, and substituted with 1-5 R₅, and —(CH₂)₀₋₄—O—(CH₂)₀₋₅-4- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CH₂)₀₋₂—NR₁₀R₁₀, C₁₋₄ alkyl substituted with 1-4 Rₑ, C₃₋₆ carbocyclyl with 1-4 Rₑ, and 3- to 6-membered heterocyclyl comprising carbon atoms and 1-4 heteroatoms selected from N, NR_{5b}, O, and S(═O)ₚ, and substituted with 1-4 Rₑ;
R₅ₐ, at each occurrence, is independently selected from the group consisting of H, C₁₋₃ alkyl substituted with 1-4 Rₑ, C(═O)R_{b}, C(═O)OR_{b}, C₃₋₆carbocyclyl substituted with 1-4 Rₑ, and 5- to 8-membered heterocyclyl comprising carbon atoms and 1-4 heteroatoms selected from the group consisting of N, NR_{5b}, O, and S(═O)ₚ, and substituted with 1-4 Rₑ;
R_{5b}, at each occurrence, is independently selected from the group consisting of H and C₁₋₃ alkyl substituted with 1-3 Rₑ;
R₆, at each occurrence, is independently selected from the group consisting of H, F, Cl, Br, CN, —NRₐRₐ, —OR_{b}, —C(═O)NRₐRₐ, —C(═O)NRₐOR_{b}, —C(═O)R_{b}, —S(═O)₂R_{c}, —NRₐS(═O)₂R_{c}, C₁₋₄ alkyl substituted with 1-4 Rₑ, —(CH₂)ᵣC₃₋₆carbocyclyl substituted with 1-4 R₇, and 5- to 6-membered heterocyclyl comprising carbon atoms and 1-4 heteroatoms selected from the group consisting of N, NR₇ₐ, O, and S(═O)ₚ, and substituted with 1-4 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚRₑ, —S(═O)ₚNRₐRₐ, C₁₋₄ alkyl substituted with 1-4 Rₑ, C₂₋₄ alkenyl substituted with 1-4 Rₑ, C₂₋₅ alkynyl substituted with 1-4 Rₑ, —(CH₂C₃₋₆carbocyclyl substituted with 1-4 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-4 R₇;
R₇, at each occurrence, is independently selected from the group consisting of H, F, Cl, Br, CN, OR_{b}, ═O, and C₁₋₃ alkyl;
R₇ₐ, at each occurrence, is independently selected from the group consisting of H and C₁₋₃ alkyl;
R₁₀, at each occurrence, is independently selected from the group consisting of H, C₁₋₄ alkyl substituted with 1-3 Rₑ, —(CH₂)ᵣ—C₃₋₁₀carbocyclyl substituted with 1-3 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H and C₁₋₃ alkyl;
R_{b}, at each occurrence, is independently selected from the group consisting of H, C₁₋₅ alkyl substituted with 1-5 Rₑ, C₃₋₁₀carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-3 R_{c};
R_{c}, at each occurrence, is independently selected from the group consisting of C₁₋₃ alkyl and C₃₋₆ cycloalkyl;
Rₑ, at each occurrence, is independently selected from the group consisting of H, F, Cl, Br, CN, ═O, C₁₋₃ alkyl substituted with 1-4 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, Br, OH, and CN.

In a fifth aspect within the scope of the fourth aspect, the present invention provides compounds of Formula (II), or stereoisomers, tautomers, pharmaceutically acceptable salts or solvates thereof, wherein:
R₂ is selected from the group consisting

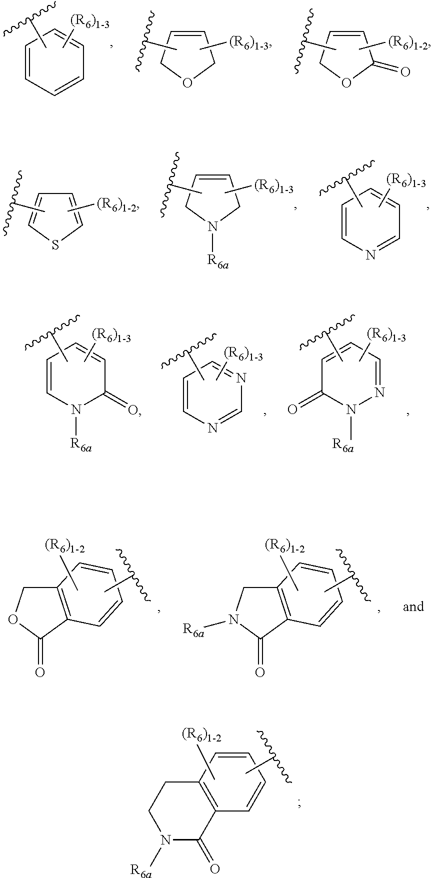

R₄′ is selected from the group consisting of F, Cl, CN, and C₁₋₃ alkyl;
R₄″ is selected from the group consisting of —O(CH₂)₁₋₃OR_{b}, —O(CH₂)₁₋₃C(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ,

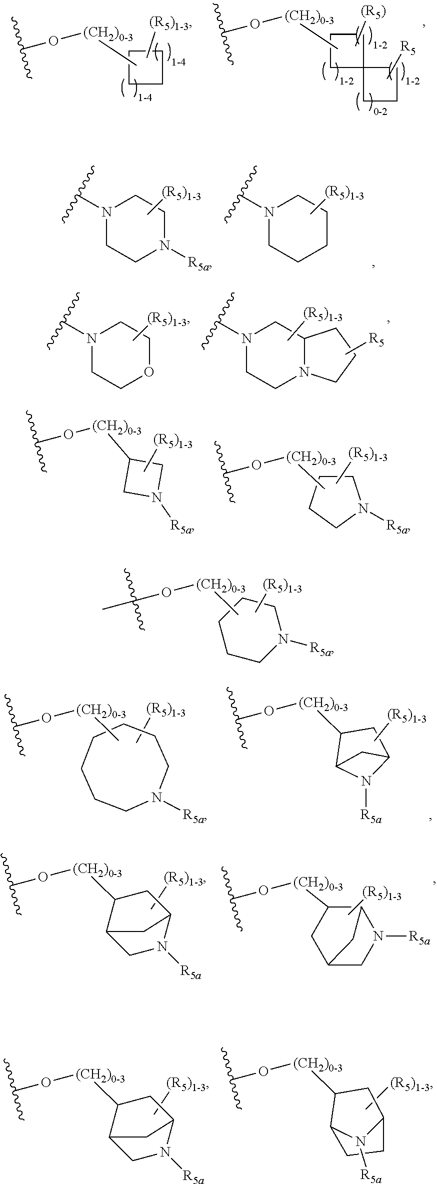

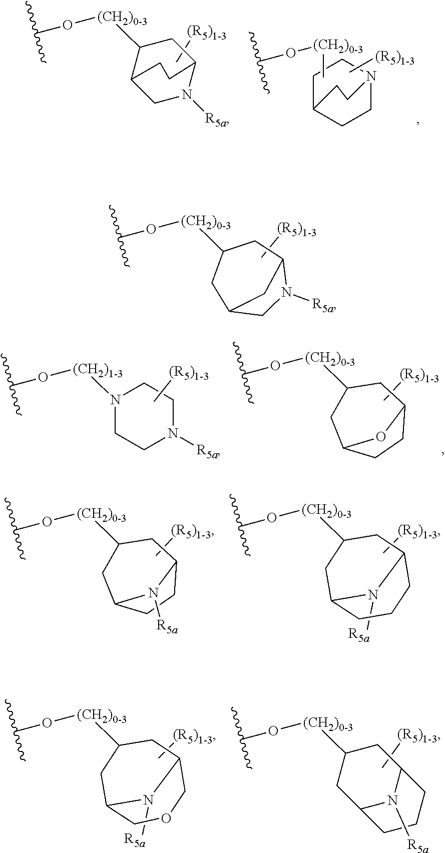

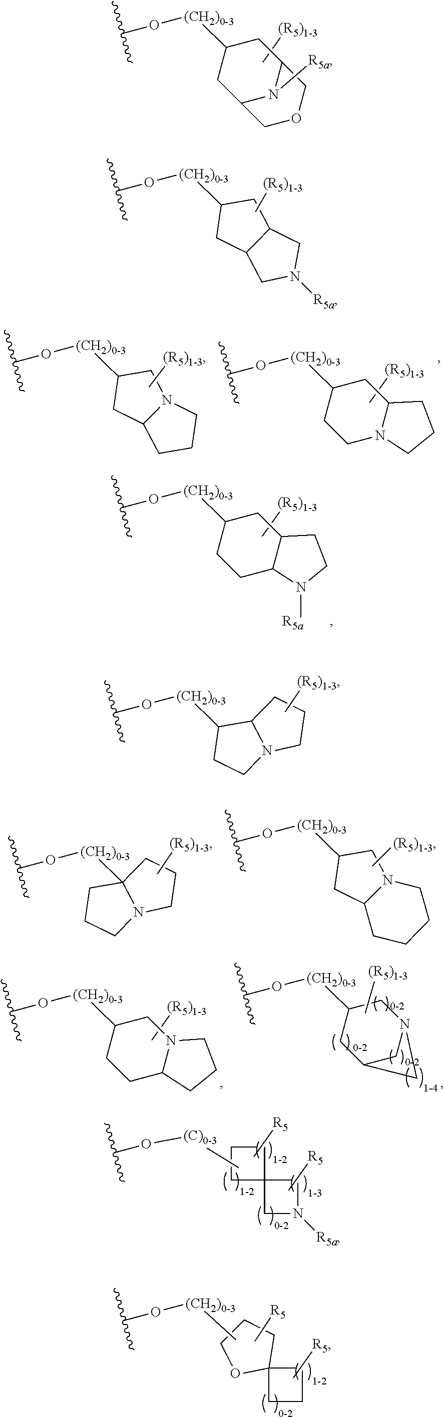

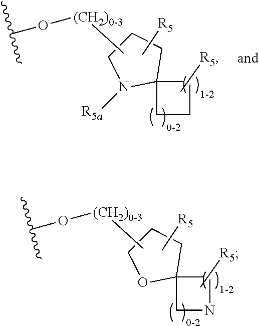

R₅, at each occurrence, is independently selected from the group consisting of H, F, Cl, Br, CN, OR_{b}, ═O, —(CH₂)₀₋₁—NR₁₀R₁₀, C₁₋₃ alkyl substituted with 1-4 Rₑ, C₃₋₆ carbocyclyl with 1-3 Rₑ, and 3- to 6-membered heterocyclyl comprising carbon atoms and 1-4 heteroatoms selected from N, NR_{5b}, O, and S(═O)ₚ, and substituted with 1-3 Rₑ;
R₅ₐ, at each occurrence, is independently selected from the group consisting of H, C₁₋₃ alkyl substituted with 1-3 Rₑ, C(═O)R_{b}, C(═O)OR_{b}, C₃₋₆carbocyclyl substituted with 1-3 Rₑ, and 3- to 10-membered heterocyclyl comprising carbon atoms and 1-4 heteroatoms selected from the group consisting of N, NR_{5b}, O, and S(═O)ₚ, and substituted with 1-3 Rₑ;
R_{5b}, at each occurrence, is independently selected from the group consisting of H and C₁₋₄ alkyl substituted with 1-3 Rₑ;
R₆, at each occurrence, is independently selected from the group consisting of H, F, Cl, Br, CN, OR_{b}, C(═O)R_{b}, —C(═O)NRₐRₐ, —S(═O)₂R_{c}, C₁₋₃ alkyl substituted with 1-5 Rₑ,

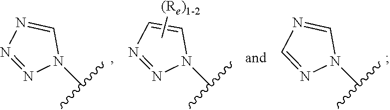

R₆ₐ, at each occurrence, is independently selected from the group consisting of —C(═O)R_{b}, —C(═O)OR_{b}, —S(═O)ₚR_{c}, C₁₋₃ alkyl substituted with 1-3 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₁₀, at each occurrence, is independently selected from the group consisting of H, C₁₋₅ alkyl substituted with 1-3 Rₑ, —(CH₂)ᵣ—C₃₋₆carbocyclyl substituted with 1-3 Rₑ, and —(CH₂)ᵣ-3- to 6-membered heterocyclyl comprising carbon atoms and 1-4 heteroatoms selected from the group consisting of N, NR_{f}, O, and S(═O)ₚ, and substituted with 1-3 Rₑ, or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H and C₁₋₃ alkyl;
R_{b}, at each occurrence, is independently selected from the group consisting of H, C₁₋₃ alky substituted with 1-4 Rₑ, C₃₋₆carbocyclyl substituted with 1-3 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
Rₑ is C₁₋₃ alky;
Rₑ, at each occurrence, is independently selected from the group consisting of H, F, Cl, CN, C₁₋₃alkyl substituted with 1-4 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, Br, OH, and CN.

In a sixth aspect within the scope of the fifth aspects, the present invention provides compounds of Formula (II), or stereoisomers, tautomers, pharmaceutically acceptable salts or solvates thereof, wherein:
R₂ is selected from the group consisting

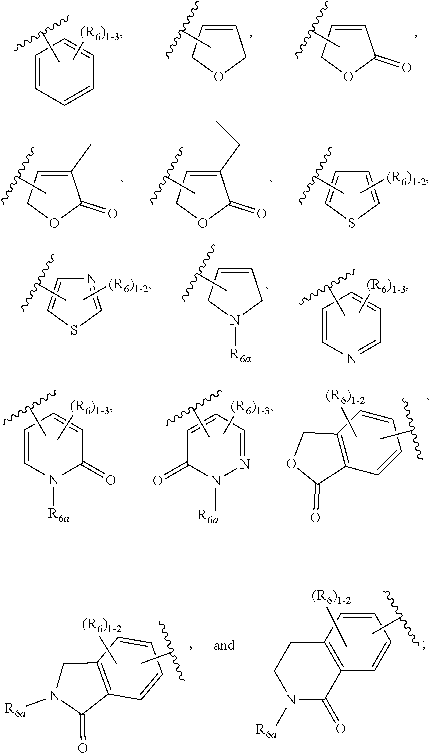

R₄′ is selected from the group consisting of F, Cl, CN, and C₁₋₃ alkyl;
R₄″ is selected from the group consisting of

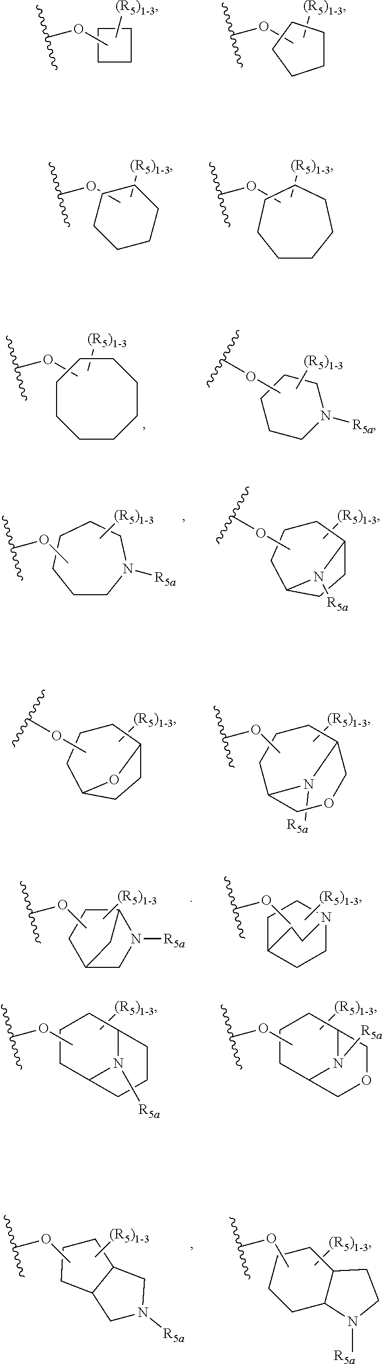

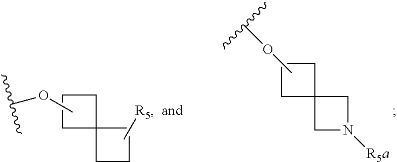

R₅, at each occurrence, is independently selected from the group consisting of H, F, Cl, Br, CN, OR_{b}, —NR₁₀R₁₀, C₁₋₃ alkyl substituted with 1-3 Rₑ, C₃₋₆ carbocyclyl, with 1-3 Rₑ, and 3- to 6-membered heterocyclyl comprising carbon atoms and 1-4 heteroatoms selected from N, NR_{5b}, O, and S(═O)ₚ, and substituted with 1-3 Rₑ;
R₅ₐ, at each occurrence, is independently selected from the group consisting of H, C₁₋₃ alkyl substituted with 1-4 Rₑ, and C(═O)R_{b};
R₆, at each occurrence, is independently selected from the group consisting of H, F, Cl, Br, CN, —S(═O)₂R_{c}, C₁₋₃ alkyl substituted with 1-4 Rₑ,

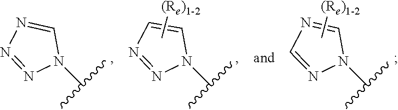

R₆ₐ, at each occurrence, is independently selected from the group consisting of —C(═O)R_{b}. —S(═O)ₚR_{c}, and C₁₋₃ alkyl substituted with 1-3 Rₑ;
R₁₀, at each occurrence, is independently selected from the group consisting of H, C₁₋₅ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H and C₁₋₃ alkyl;
R_{b}, at each occurrence, is independently selected from the group consisting of H C₁₋₃ alky substituted with 1-3 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
R_{c} is C₁₋₃ alky;
Rₑ, at each occurrence, is independently selected from the group consisting of H, F, Cl, CN, C₁₋₃ alkyl substituted with 1-3 R_{g}, NR_{f}R_{f}, and —OR_{f};
R_{f} is independently selected from the group consisting of H and C₁₋₃ alkyl; and
Rₐ is independently selected from the group consisting of H, F, Cl, Br, OH, and CN.

In a seventh aspect within the scope of the sixth aspect, the present invention provides compounds of Formula (II), or stereoisomers, tautomers, pharmaceutically acceptable salts or solvates thereof, wherein:
R₄″ is selected from the group consisting of

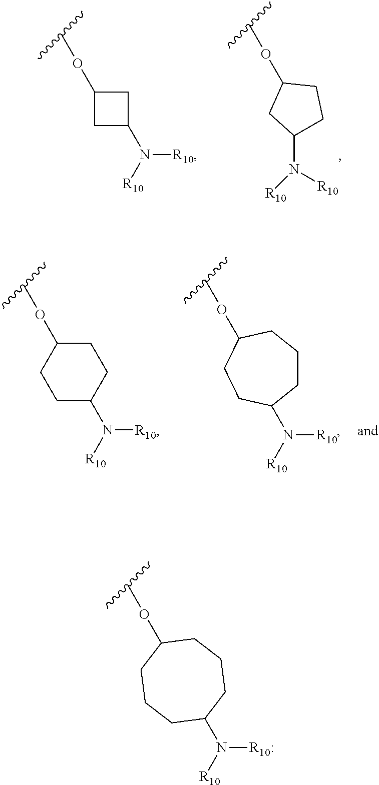

R₁₀, at each occurrence, is independently selected from the group consisting of H, C₁₋₅ alkyl substituted with 1-3 Rₑ, —(CH₂)₀₋₁—C₃₋₆ cycloalkyl substituted with 1-3 Rₑ, and 3- to 6-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a —(CH₂)₀₋₁3- to 6-membered heterocyclyl comprising carbon atoms and 1-4 heteroatoms selected from the group consisting of N, NR_{f}, and O, and substituted with 1-3 Rₑ;
Rₑ, at each occurrence, is independently selected from the group consisting of H, F, Cl, CN, C₁₋₃ alkyl substituted with 1-3 R_{g}, and —OR_{f};
R_{f} is independently selected from the group consisting of H and C₁₋₃ alkyl,
R_{g}, at each occurrence, is independently selected from the group consisting of H, F, Cl, —OH, and CN.

In an eighth aspect within the scope of the seventh aspect, the present invention provides compounds of Formula (II), or stereoisomers, tautomers, pharmaceutically acceptable salts or solvates thereof, wherein:
R₁₀, at each occurrence, is independently selected from the group consisting of H, Me, Et,

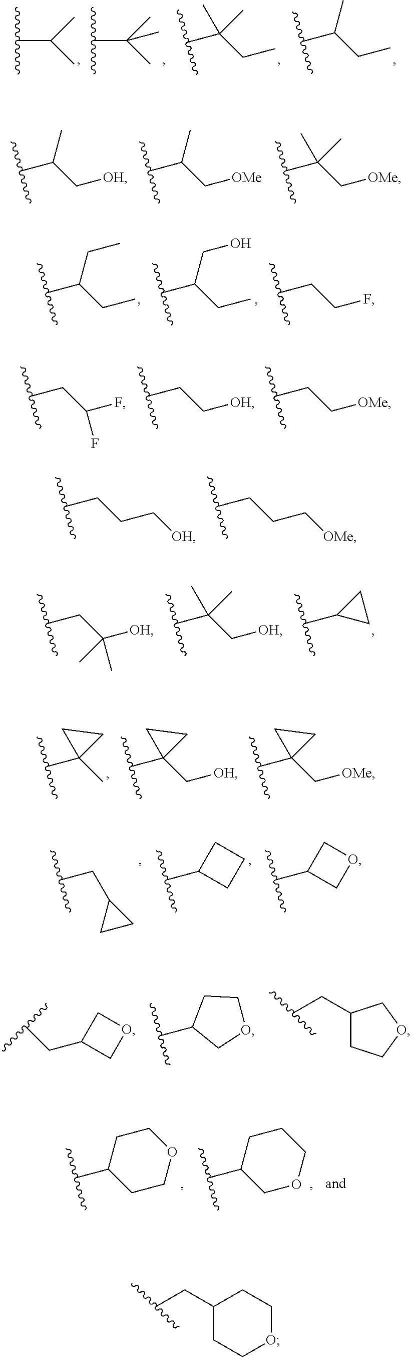

or R₁₀ and R₁₀ together with the nitrogen atom to which they are both attached form a heterocyclyl selected from the group consisting of

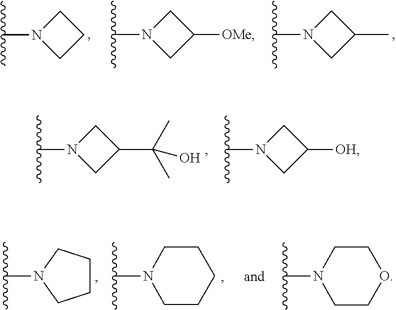

In a ninth aspect within the scope of the second or fourth aspect, the present invention provides compounds of Formula (II), or stereoisomers, tautomers, pharmaceutically acceptable salts or solvates thereof, wherein:
R₁ is selected from the group consisting of

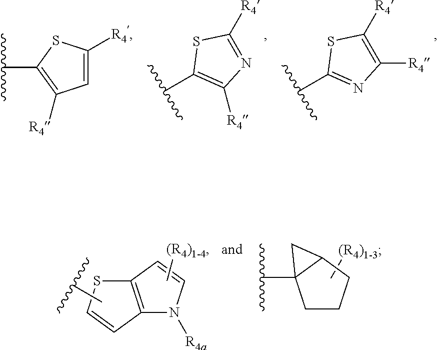

R₂ is selected from the group consisting of

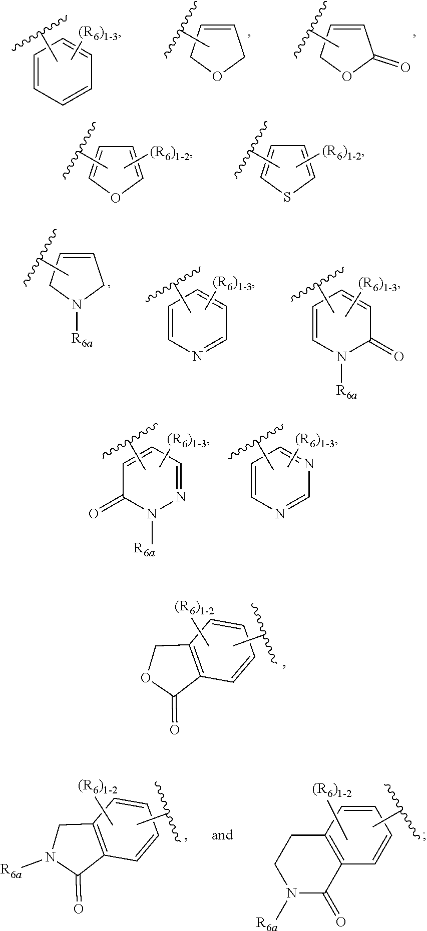

R₄, at each occurrence, is independently selected from the group consisting of H, F, Cl, Br, ═O, and CN;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, and C₁₋₅ alkyl substituted with 1-5 Rₑ;
R₄′ is selected from the group consisting of F, Cl, CN, and C₁₋₃ alkyl substituted with 1-3 Rₑ;
R₄″ is selected from the group consisting of —OC₁₋₄ alkyl substituted with 1-2 Rₑ, —O(CH₂)₁₋₃NRₐRₐ,

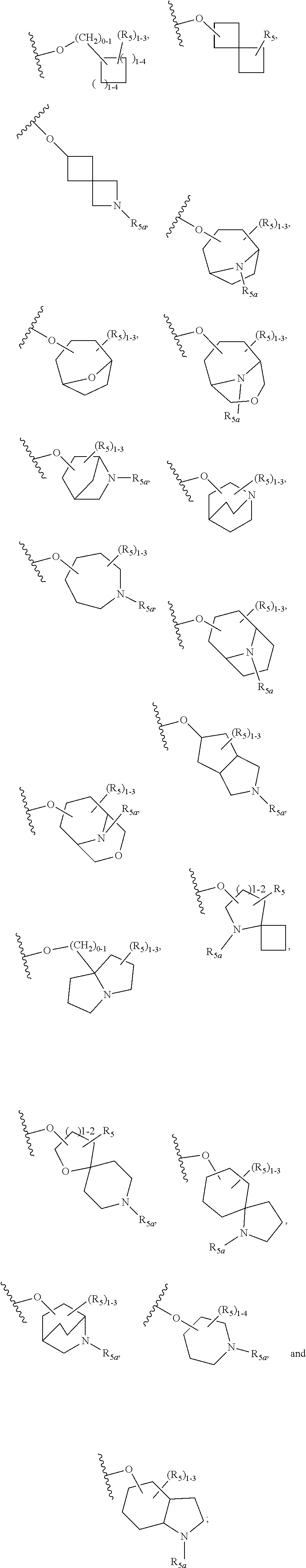

R₅, at each occurrence, is independently selected from the group consisting of H, F, Cl, Br, CN, OR_{b}, —(CH₂)₀₋₁—NR₁₀R₁₀, C₁₋₃ alkyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₃ alkyl substituted with 1-4 Rₑ, C(═O)R_{b}, and C₃₋₆ cycloalkyl;
R_{b}, at each occurrence, is independently selected from the group consisting of H, F, Cl, Br, CN, OR_{b}, —S(═O)₂R_{c}, C₁₋₃ alkyl substituted with 1-5 Rₑ,

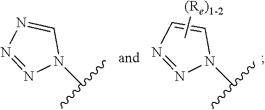

R₆ₐ, at each occurrence, is independently selected from the group consisting of —C(═O)R_{b}, —S(═O)ₚR_{c}, and C₁₋₃ alkyl substituted with 1-3 Rₑ;
R₁₀, at each occurrence, is independently selected from the group consisting of H, C₁₋₅ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, and O, and substituted with 1-3 Rₑ;
Rₐ, at each occurrence, is independently selected from the group consisting of H, C₁₋₄ alkyl substituted with 1-2 Rₑ, C₃₋₆ carbocyclyl, and 3- to 6-membered heterocyclyl comprising carbon atoms and 1-4 heteroatoms selected from the group consisting of N, NR_{f}, O, and S(═O)ₚ;
R_{b}, at each occurrence, is independently selected from the group consisting of H, C₁₋₃ alky substituted with 1-3 Rₑ, C₃₋₆ carbocyclyl substituted with 1-2 Rₑ, and 5- to 6-membered heterocyclyl comprising carbon atoms and 1-4 heteroatoms selected from the group consisting of N, NR_{f}, O, and S(═O)ₚ, and substituted with 1-2 Rₑ;
R_{c} is C₁₋₃ alky;
Rₑ, at each occurrence, is independently selected from the group consisting of H, F, Cl, CN, C₁₋₃ alkyl substituted with 1-3 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, Br, OH, and CN.

In a tenth aspect within the scope of the second aspect, the present invention provides compounds of Formula (III):

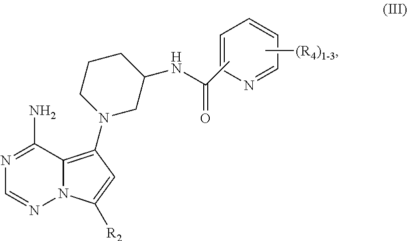

or stereoisomers, tautomers, pharmaceutically acceptable salts or solvates thereof,
wherein:
R₂ is selected from the group consisting of

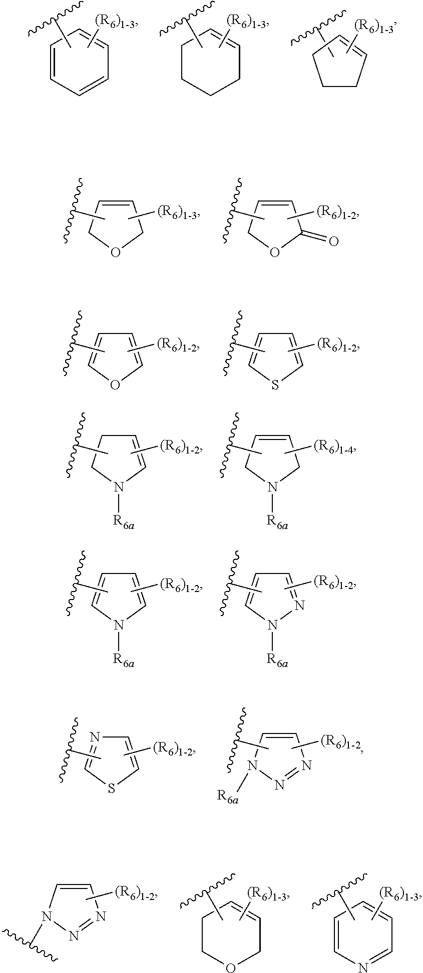

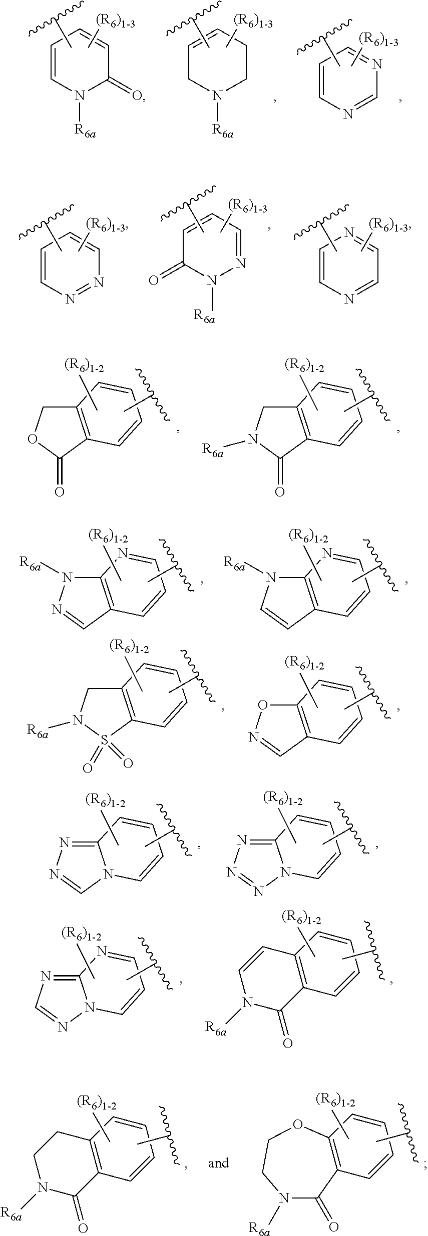

R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₁₀carbocyclyl substituted with 1-5 R₅, —(CH₂)ᵣ—O—(CH₂)ᵣ—C₃₋₁₀carbocyclyl, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CH₂)ᵣ—NR₁₀R₁₀, C₁₋₅ alkyl substituted with 1-4 Rₑ, C₃₋₆ carbocyclyl with 1-4 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H and C₁₋₅ alkyl substituted with 1-4 Rₐ, C(═O)R_{b}, C(═O)OR_{b}, C₃₋₆carbocyclyl substituted with 1-4 Rₑ, and heterocyclyl comprising carbon atoms and 1-4 heteroatoms selected from the group consisting of N, NR_{5b}, O, and S(═O)ₚ, and substituted with 1-4 Rₑ;
R_{5b}, at each occurrence, is independently selected from the group consisting of H and C₁₋₄ alkyl substituted with 1-4 Rₑ;
R₆, at each occurrence, is independently selected from the group consisting of H, F, Cl, Br, CN, ═O, —(CH₂)ᵣNRₐRₐ, NO₂, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂R_{c}, —NRₐS(═O)₂R_{c}, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₆ alkynyl substituted with 1-5 Rₑ, —(CH₂)ᵣC₃₋₆carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ, at each occurrence, is independently selected from the group consisting of H and C₁₋₄ alkyl substituted with 1-5 Rₑ;
R₁₀, at each occurrence, is independently selected from the group consisting of H, C₁₋₅ alkyl substituted with 1-4 Rₑ, —(CH₂)ᵣ—C₃₋₁₀carbocyclyl substituted with 1-4 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-4 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-4 Rₑ;
Rₐ, at each occurrence, is independently selected from the group consisting of H, C₁₋₅ alkyl substituted with 1-4 Rₑ, C₃₋₁₀carbocyclyl substituted with 1-4 Rₑ, and —(CH₂)ᵣ-heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 1-4 Rₑ, C₂₋₅ alkynyl substituted with 1-4 Rₑ, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c}, at each occurrence, is independently selected from the group consisting of C₁₋₅ alkyl substituted with 1-4 Rₑ, C₃₋₆carbocyclyl substituted with 1-4 Rₑ, and heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H C₁₋₅ alkyl, and C₃₋₆ cycloalkyl;
Rₑ, at each occurrence, is independently selected from the group consisting of H, F, Cl, Br, CN, ═O, C₁₋₅alkyl substituted with 1-4 R_{g}, C₂₋₅ alkenyl substituted with 1-4 R_{g}, C₂₋₅ alkynyl substituted with 1-4 R_{g}, —(CH₂)ᵣ—C₃₋₆ cycloalkyl, —(CH₂)ᵣ-heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a heterocyclic ring;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4, or 5.

In an eleventh aspect within the scope of the second aspect, the present invention provides compounds of Formula (II), or stereoisomers, tautomers, pharmaceutically acceptable salts or solvates thereof, wherein:
R₁ is selected from the group consisting of

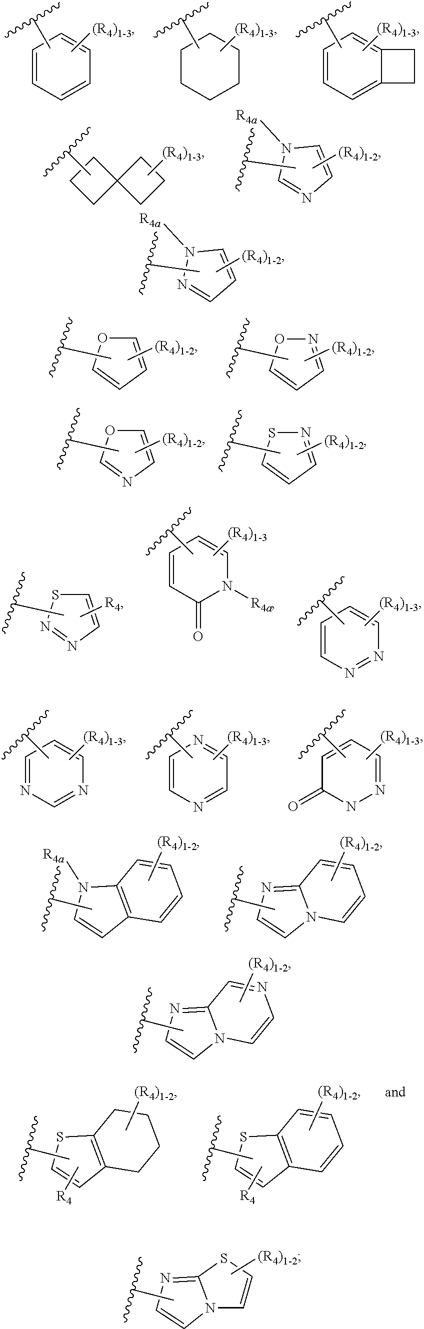

R₂ is selected from the group consisting of

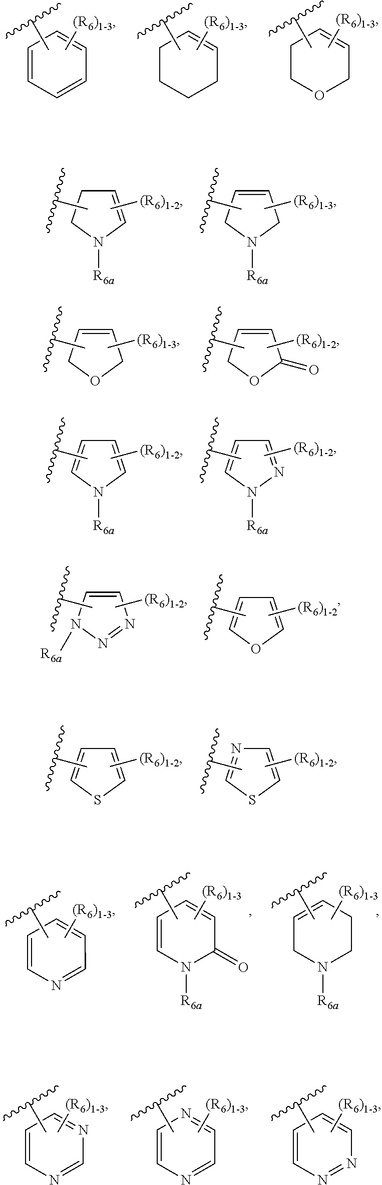

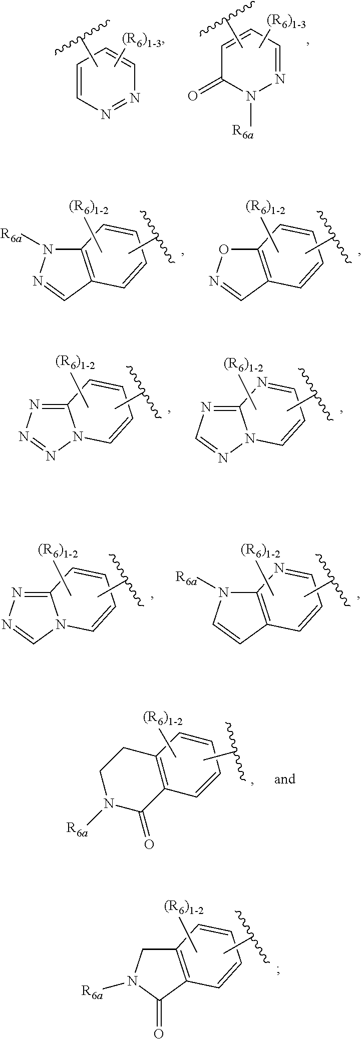

R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 N, C₂₋₄ alkenyl substituted with 1-5 Rₑ, and C₂₋₄ alkynyl substituted with 1-5 R₅, —(CH₂)ᵣ—C₃₋₁₀carbocyclyl substituted with 1-5 R₅, —(CH₂)ᵣ—O—(CH₂)ᵣ—C₃₋₁₀carbocyclyl, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H and C₁₋₄ alkyl substituted with 1-5 Rₑ;
R₅, at each occurrence, is independently selected from the group consisting of H, Cl, Br, CN, OR_{b}, ═O, —(CH₂)₀₋₂—NR₁₀R₁₀, C₁₋₅ alkyl substituted with 1-3 Rₑ, C₂₋₆ alkenyl substituted with 1-5 Rₑ, C₂₋₆ alkynyl substituted with 1-3 Rₑ, C₃₋₆ carbocyclyl with 1-3 Rₑ, and 3- to 6-membered heterocyclyl comprising carbon atoms and 1-4 heteroatoms selected from N, NR_{5b}, O, and S(═O)ₚ, and substituted with 1-4 Rₑ;
R₅ₐ, at each occurrence, is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₆ alkyl substituted with 1-5 Rₑ;
R₆, at each occurrence, is independently selected from the group consisting of H, F, Cl, OR_{b}, —S(═O)₂Rₑ, and 5- to 6-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-4 Rₑ, C₂₋₄ alkenyl substituted with 1-5 Rₑ, C₂₋₄ alkynyl substituted with 1-4 Rₑ, —(CH₂)ᵣC₃₋₆ carbocyclyl substituted with 1-4 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-4 R₇;
R₇, at each occurrence, is independently selected from the group consisting of H, F, Cl, Br, OR_{b}, ═O, CN, and C₁₋₃ alkyl;
R₇ₐ, at each occurrence, is independently selected from the group consisting of H and C₁₋₃ alkyl;
R₁₀, at each occurrence, is independently selected from the group consisting of H, C₁₋₄ alkyl substituted with 1-3 Rₑ, —(CH₂)ᵣ—C₃₋₁₀carbocyclyl substituted with 1-3 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H, C(═O)OR_{b}, C₁₋₅ alkyl substituted with 1-4 Rₑ, C₃₋₁₀carbocyclyl substituted with 1-4 Rₑ, and —(CH₂)ᵣ-heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 14 Rₑ, C₂₋₅ alkynyl substituted with 1-4 Rₑ, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c}, at each occurrence, is independently selected from the group consisting of C₁₋₅ alkyl substituted with 1-4 Rₑ, C₃₋₆carbocyclyl substituted with 1-4 Rₑ, and heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H C₁₋₄ alkyl, and C₃₋₆ cycloalkyl;
Rₑ, at each occurrence, is independently selected from the group consisting of H, F, Cl, Br, CN, ═O, C₁₋₅alkyl substituted with 1-4 R_{g}, C₂₋₅ alkenyl substituted with 1-4 R_{g}, C₂₋₅ alkynyl substituted with 1-4 R_{g}, —(CH₂)ᵣ—C₃₋₆ cycloalkyl, —(CH₂)ᵣ-heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a heterocyclic ring;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4, or 5.

In a twelfth aspect within the scope of the eleventh aspect, the present invention provides compounds of Formula (II), or stereoisomers, tautomers, pharmaceutically acceptable salts or solvates thereof, wherein:
R₂ is selected from the group consisting of

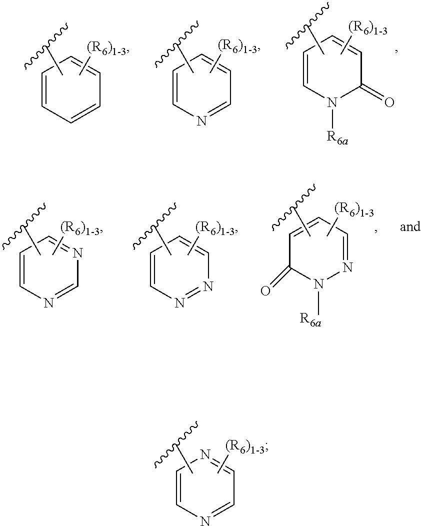

R₅, at each occurrence, is independently selected from the group consisting of H, F, Cl, CN, OR_{b}, C(═O)R_{b}, —C(═O)NRₐRₐ, —S(═O)₂R_{c} C₁₋₃ alkyl substituted with 1-5 Rₑ,

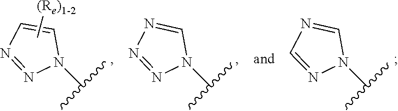

R₆ₐ, at each occurrence, is independently selected from the group consisting of H, C(═O)R_{b}, C(═O)OR_{b}, and C₁₋₃ alkyl substituted with 1-5 Rₑ;
Rₐ, at each occurrence, is independently selected from the group consisting of H, C₁₋₃ alkyl, and C(═O)OR_{b};
R_{b}, at each occurrence, is independently selected from the group consisting of H C₁₋₃ alkyl substituted with 1-5 Rₑ, C₃₋₁₀carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c} is C₁₋₃ alkyl;
Rₑ, at each occurrence, is independently selected from the group consisting of H, F, Cl, CN, and OR_{f}; and
R_{f}, at each occurrence, is independently selected from the group consisting of H and C₁₋₃ alkyl.

In a thirteenth aspect within the scope of the eleventh aspect, the present invention provides compounds of Formula (II), or stereoisomers, tautomers, pharmaceutically acceptable salts or solvates thereof, wherein:
R₂ is selected from the group consisting of

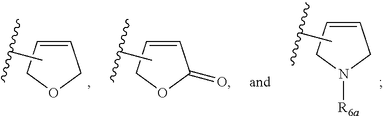

R₆ₐ, is selected from the group consisting of H, C(═O)R_{b}, C(═O)OR_{b}, —S(═O)₂R_{c}, and C₁₋₃ alkyl substituted with 1-5 Rₑ;
R_{b}, at each occurrence, is independently selected from the group consisting of H, C₁₋₃ alkyl, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c} is C₁₋₃ alkyl;
Rₑ, at each occurrence, is independently selected from the group consisting of H, F, Cl, CN, and OR_{f}, and
R_{f}, at each occurrence, is independently selected from the group consisting of H and C₁₋₃ alkyl.

In a fourteenth aspect within the scope of the eleventh aspect, the present invention provides compounds of Formula (II), or stereoisomers, tautomers, pharmaceutically acceptable salts or solvates thereof, wherein:
R₁ is selected from the group consisting of

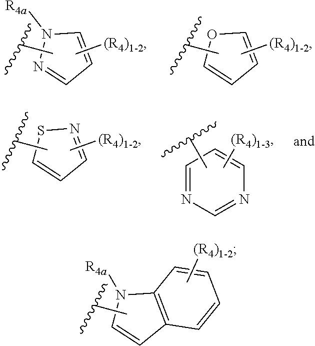

R₂ is selected from the group consisting of

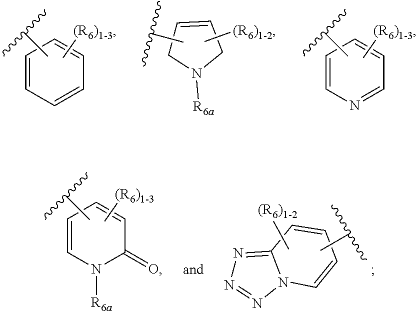

R₄, at each occurrence, is independently selected from the group consisting of H F, Cl, —OR_{b}, —O(CH₂)₁₋₄NRₐRₐ, C₁₋₃ alkyl substituted with 1-5 Rₑ, C₃₋₆ carbocyclyl substituted with 1-5 R₅, —O—(CH₂)ᵣ—C₃₋₆ carbocyclyl substituted with 1-5 R₅, 4- to 10-membered heterocyclyl substituted with 1-5 R₅, and —O—(CH₂)ᵣ-4- to 10-membered heterocyclyl substituted with 1-5 R₅, wherein the C₃₋₆carbocyclyl is selected from the group consisting of C₃₋₆ cycloalkyl and phenyl and the heterocyclyl is selected from the group consisting of

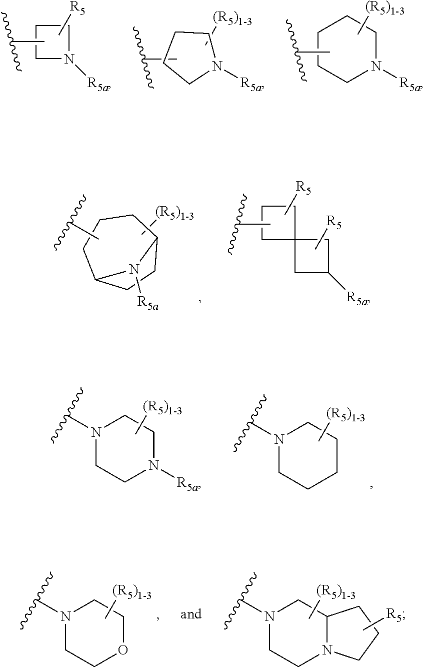

R₄ₐ, at each occurrence, is independently selected from the group consisting of H and C₁₋₃ alkyl;
R₅, at each occurrence, is independently selected from the group consisting of H, F, Cl, Br, CN, OH, ═O, —NR₁₀R₁₀, C₁₋₃ alkyl substituted with 1-5 Rₑ, —OC₁₋₅ alkyl substituted with 1-5 Rₑ, and C₃₋₆ cycloalkyl substituted with 1-5 Rₑ;
R₅ₐ, at each occurrence, is independently selected from the group consisting of H, C₁₋₃ alkyl substituted with 1-4 Rₑ, C(═O)R_{b}, and C(═O)OR_{b};
R_{5b}, at each occurrence, is independently selected from the group consisting of H and C₁₋₆ alkyl substituted with 1-5 Rₑ;
R₆, at each occurrence, is independently selected from the group consisting of H, F, Cl, Br, CN, OR_{b}, C(═O)R_{b}, —C(═O)NRₐRₐ, —S(═O)₂R_{c}, C₁₋₃ alkyl substituted with 1-5 Rₑ,

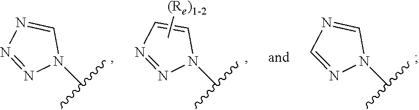

R₆ₐ, at each occurrence, is independently selected from the group consisting of H, C(═O)R_{b}, C(═O)OR_{b}, and C₁₋₃ alkyl substituted with 1-5 Rₑ;
R₁₀, at each occurrence, is independently selected from the group consisting of H and C₁₋₄ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H, C₁₋₃ alkyl, and C(═O)OR_{b};
R_{b}, at each occurrence, is independently selected from the group consisting of H, C₁₋₃ alkyl, and

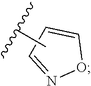

R_{c}, at each occurrence, is C₁₋₃ alkyl substituted with 1-5 Rₑ;
Rₑ, at each occurrence, is independently selected from the group consisting of H, F, Cl, CN, and OR_{f};
R_{f}, at each occurrence, is independently selected from the group consisting of H and C₁₋₃ alkyl; and
r is an integer of zero or 1.

In a fifteenth aspect within the scope of the second aspect, the present invention provides compounds of Formula (IV):

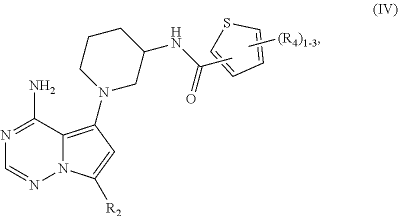

or stereoisomers, tautomers, pharmaceutically acceptable salts or solvates thereof,
wherein:
R₂ is independently selected from the group consisting of

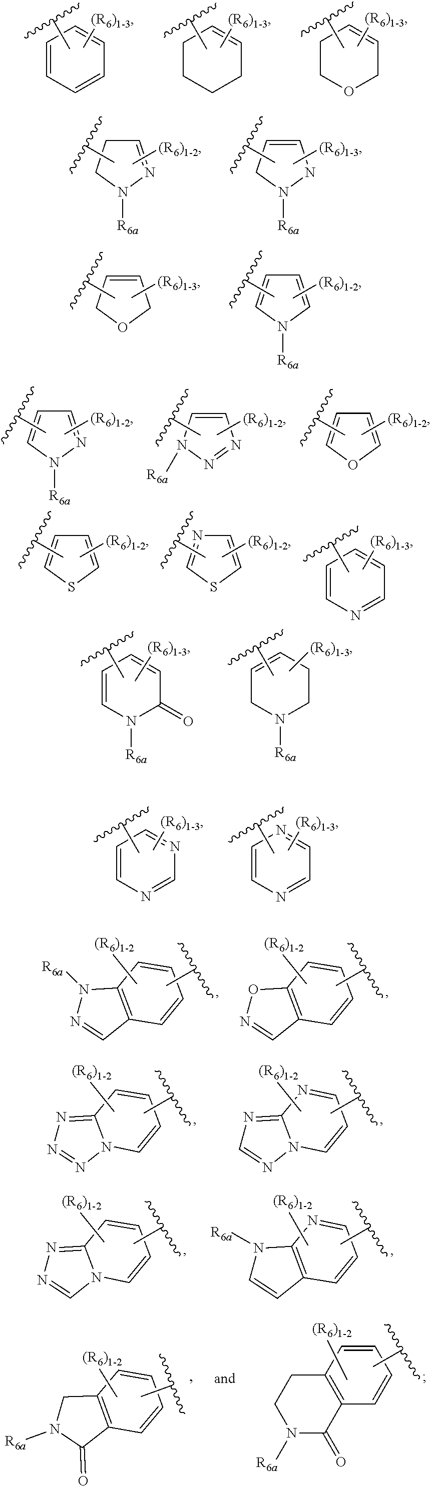

R₄ is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ-3-6carbocyclyl substituted with 1-5 R₅, —(CH₂)ᵣ-4- to 16-membered heterocyclyl comprising carbon atoms and 1-4 heteroatoms selected from N, NR₅ₐ, O, and S(═O)ₚ, and substituted with 1-5 R₅, and —(CH₂)—O—(CH₂)ᵣ-4- to 16-membered heterocyclyl comprising carbon atoms and 1-4 heteroatoms selected from N, NR₅ₐ, O, and S(═O)ₚ, and substituted with 1-5 R₅;
R₅ is independently selected from the group consisting of H, F, Cl, Br, CN, OR_{b}, ═O, —NR₁₀R₁₀, and C₁₋₅ alkyl substituted with 1-5 Rₑ, C₃₋₆ carbocyclyl with 1-5 Rₑ, and 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H and C₁₋₅ alkyl substituted with 1-4 Rₑ, C(═O)R_{b}, C(═O)OR_{b}, C₃₋₆carbocyclyl substituted with 1-4 Rₑ, and heterocyclyl comprising carbon atoms and 1-4 heteroatoms selected from the group consisting of N, NR_{5b}, O, and S(═O)ₚ, and substituted with 1-4 Rₑ;
R_{5b} is independently selected from the group consisting of H and C₁₋₄ alkyl substituted with 1-4 Rₑ;
R₆ is independently selected from the group consisting of H, F, Cl, Br, CN, ═O, —(CH₂)ᵣNRₐRₑ, NO₂, —OR_{b}, —C(═O)NRₐRₐ, —C(═O)NRₐ(CH₂)R_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂R_{c}, —NRₐS(═O)₂Rₑ, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₆ alkynyl substituted with 1-5 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇ is independently selected from the group consisting of H, F, Cl, Br, OR_{b}, ═O, CN, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ is independently selected from the group consisting of H and C₁₋₄ alkyl substituted with 1-5 Rₑ;
R₁₀, at each occurrence, is independently selected from the group consisting of H, C₁₋₅ alkyl substituted with 1-4 Rₑ, —(CH₂)ᵣ—C₃₋₁₀carbocyclyl substituted with 1-4 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-4 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-4 Rₑ;
Rₐ is independently selected from the group consisting of H, C₁₋₅ alkyl substituted with 1-4 Rₑ, C₃₋₁₀carbocyclyl substituted with 1-4 Rₑ, and —(CH₂)ᵣ-heterocyclyl comprising carbon atoms and 1-4 heteroatoms selected from the group consisting of N, O, and S(═O)ₚ, and substituted with 1-5 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-5 Rₑ;
R_{b} is independently selected from the group consisting of H, C₁₋₅ alkyl substituted with 1-5 Rₑ, C₂₋₅ alkenyl substituted with 1-5 Rₑ, C₂₋₅ alkynyl substituted with 1-5 Rₑ, C₃₋₁₀carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
R_{c} is independently selected from the group consisting of C₁₋₅ alkyl substituted with 1-5 Rₑ, C₃₋₆carbocyclyl substituted with 1-5 Rₑ, and heterocyclyl comprising carbon atoms and 1-4 heteroatoms selected from the group consisting of N, NR_{f}, O, and S(═O)ₚ, and substituted with 1-5 Rₑ;
R_{d} is independently selected from the group consisting of H, C₁₋₄ alkyl and C₃₋₆ cycloalkyl;
Rₑ is independently selected from the group consisting of H, F, Cl, Br, CN, ═O, C₁₋₅ alkyl substituted with 1-4 R_{g}, C₂₋₅ alkenyl substituted with 1-4 R_{g}, C₂₋₅alkynyl substituted with 1-4 R_{g}, —(CH₂)ᵣ—C₃₋₆ cycloalkyl, —(CH₂)ᵣ-heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, —(CH₂)ᵣC(═O)R₄, and —(CH₂)ᵣC(═O)OR_{f};
R_{f} is independently selected from the group consisting of H, C₁₋₅ alkyl (optionally substituted with F, Cl, Br, OH, NH₂), C₃₋₆ cycloalkyl; or R_{f} and R_{f} together with the nitrogen atom to which they are both attached form a heterocyclic ring;
R_{g} is independently selected from the group consisting of H, F, Cl, Br, OH, C₁₋₅ alkyl, and C₃₋₆ cycloalkyl;
p is an integer of zero, 1, or 2; and
r is an integer of zero, 1, 2, 3, 4, or 5.

In a sixteenth aspect within the scope of the fifteenth aspect, the present invention provides compounds of Formula (IV), or stereoisomers, tautomers, pharmaceutically acceptable salts or solvates thereof, wherein:

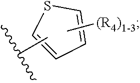

is

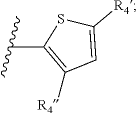

R₂ is independently selected from the group consisting

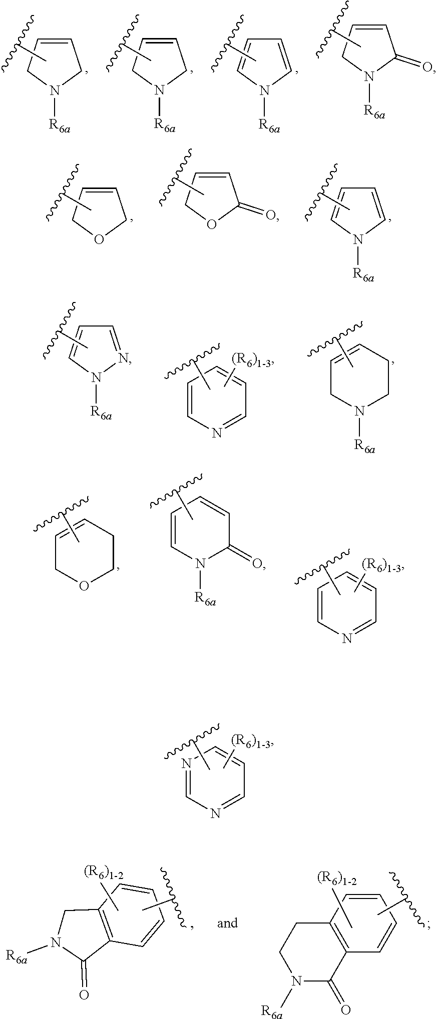

R₄′ is independently selected from the group consisting of H, F, Cl, CN, and C₁₋₄ alkyl substituted with 1-3 Rₑ;
R₄″ is independently selected from the group consisting of —O(CH₂)₁₋₃OR_{b}, —O(CH₂)₁₋₃C(═O)NRₐRₐ, —O(CH₂)ᵣNRₐC(═O)R_{b}, —O(CH₂)ᵣNRₐC(═O)OR_{b}, —O(CH₂)₁₋₃NRₐRₐ, and —O—C₃₋₆ cycloalkyl substituted with 1-5 R₅,

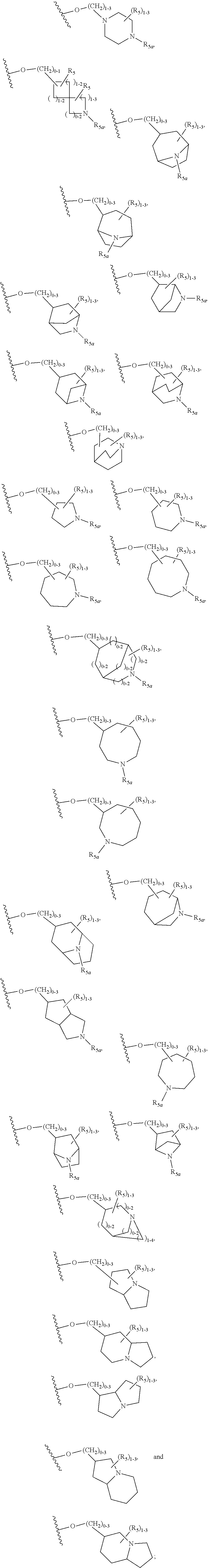

R₅ is independently selected from the group consisting of H, F, Cl, Br, CN, OR_{b}, ═O, C₁₋₃ alkyl substituted with 1-4 Rₑ, C₃₋₆ carbocyclyl with 1-4 Rₑ, 3- to 6-membered heterocyclyl comprising 1-4 heteroatoms selected from N, NR_{5b}, O, and S(═O)ₚ, and substituted with 1-4 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-4 Rₑ, and 3- to 10-membered heterocyclyl comprising carbon atoms and 1-4 heteroatoms selected from the group consisting of N, NR_{5b}, O, and S(═O)ₚ, and substituted with 1-4 Rₑ;
R_{5b} is independently selected from the group consisting of H and C₁₋₅ alkyl substituted with 1-4 Rₑ;
R₆ is independently selected from the group consisting of H, F, Cl, Br, CN, OR_{b}, C(═O)R_{b}, —C(═O)NRₐRₐ, —S(═O)₂R_{c}, C₁₋₃ alkyl substituted with 1-5 Rₑ,

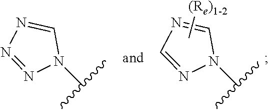

R₆ₐ is independently selected from the group consisting of H, —C(═O)NRₐRₐ, —C(═O)R_{b}, —C(═O)(CH₂)ᵣOR_{b}, —S(═O)ₚR₄, and C₁₋₃ alkyl substituted with 1-3 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
Rₐ is independently selected from the group consisting of H and C₁₋₃ alkyl;
R_{b} is independently selected from the group consisting of H, C₁₋₃ alkyl, C₃₋₁₀carbocyclyl substituted with 1-3 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
R_{c} is independently selected from the group consisting of C₁₋₃ alkyl substituted with 1-3 Rₑ,
Rₑ is independently selected from the group consisting of H, F, Cl, Br, CN, C₁₋₃ alkyl, and —OR_{f}; and
R_{f} is independently selected from the group consisting of H and C₁₋₃ alkyl.

In a seventeenth aspect within the scope of the second aspect, the present invention provides compounds of Formula (V):

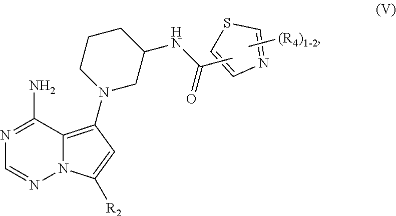

or stereoisomers, tautomers, pharmaceutically acceptable salts or solvates thereof,
wherein:
R₂ is independently selected from the group consisting of

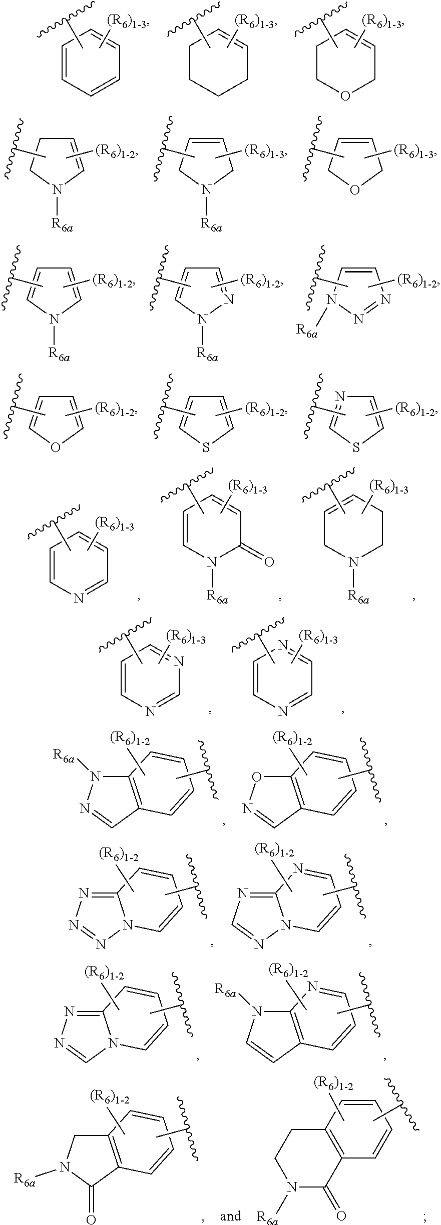

R₄ is independently selected from the group consisting of H, F, Cl, Br, ═O, CN, —(CH₂)ᵣNRₐRₐ, —OR_{b}, —O(CH₂)₁₋₄OR_{b}, —O(CH₂)ᵣC(═O)NRₐRₐ, —O(CH₂)₁₋₄NRₐC(═O)R_{b}, —O(CH₂)₁₋₄NRₐC(═O)OR_{b}, —O(CH₂)₁₋₄NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₆ carbocyclyl substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and (CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₅ is independently selected from the group consisting of H, F, Cl, Br, CN, OR_{b}, ═O, —NRₐRₐ, C₁₋₅ alkyl substituted with 1-5 Rₑ, C₃₋₆ carbocyclyl with 1-5 Rₑ, and 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₁₋₆ alkyl substituted with 1-5 Rₑ;
R₆ is independently selected from the group consisting of H, F, Cl, Br, CN, ═O, —(CH₂)ᵣNRₐRₐ, NO₂, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂R_{c}, —NRₐS(═O)₂R_{c}, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₆ alkynyl substituted with 1-5 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇ is independently selected from the group consisting of H, F, Cl, Br, OR_{b}, ═O, CN, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ is independently selected from the group consisting of H and C₁₋₄ alkyl substituted with 1-5 Rₑ;
Rₐ is independently selected from the group consisting of H, C₁₋₅ alkyl substituted with 1-4 Rₑ, C₃₋₁₀carbocyclyl substituted with 1-4 Rₑ, and —(CH₂)ᵣ-heterocyclyl comprising carbon atoms and 1-4 heteroatoms selected from the group consisting of N, O, and S(═O)ₚ, and substituted with 1-5 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-5 Rₑ;
R_{b} is independently selected from the group consisting of H, C₁₋₅ alkyl substituted with 1-5 Rₑ, C₂₋₅ alkenyl substituted with 1-5 Rₑ, C₂₋₅ alkynyl substituted with 1-5 Rₑ, C₃₋₁₀carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
Rₑ is independently selected from the group consisting of C₁₋₅ alkyl substituted with 1-5 Rₑ, C₂₋₅ alkenyl substituted with 1-5 Rₑ, C₂₋₅ alkynyl substituted with 1-5 Rₑ, C₃₋₆ carbocyclyl substituted with 1-5 Rₑ, and heterocyclyl comprising carbon atoms and 1-4 heteroatoms selected from the group consisting of N, NR_{f}, O, and S(═O)ₚ, and substituted with 1-5 Rₑ;
R_{d} is independently selected from the group consisting of H, C₁₋₄ alkyl and C₃₋₆ cycloalkyl;
Rₑ is independently selected from the group consisting of H, F, Cl, Br, CN, ═O, C₁₋₅ alkyl substituted with 1-4 R_{g}, C₂₋₅ alkenyl substituted with 1-4 R_{g}, C₂₋₅ alkynyl substituted with 1-4 R_{g}, —(CH₂)ᵣC₃₋₆ cycloalkyl, —(CH₂)ᵣ-heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, (CH₂)ᵣC(═O)R_{f}, and —(CH₂)ᵣC(═O)OR_{f};
R_{f} is independently selected from the group consisting of H, C₁₋₅ alkyl (optionally substituted with F, Cl, Br, OH, NH₂), C₃₋₆ cycloalkyl; or R_{f} and R_{f} together with the nitrogen atom to which they are both attached form a heterocyclic ring;
R_{g} is independently selected from the group consisting of H, F, Cl, Br, OH, C₁₋₅ alkyl, and C₃₋₆ cycloalkyl;
p is an integer independently selected from the group consisting of zero, 1, and 2; and
   r is an integer independently selected from the group consisting of zero, 1, 2, 3, and 4.

In an eighteenth aspect within the scope of the seventh aspect, the present invention provides compounds of Formula (VI):

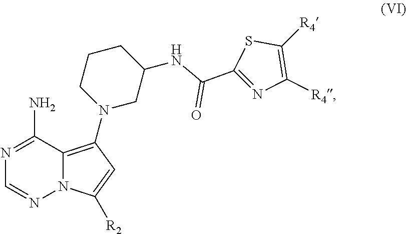

or stereoisomers, tautomers, pharmaceutically acceptable salts or solvates thereof,
wherein:
R₂ is independently selected from the group consisting of

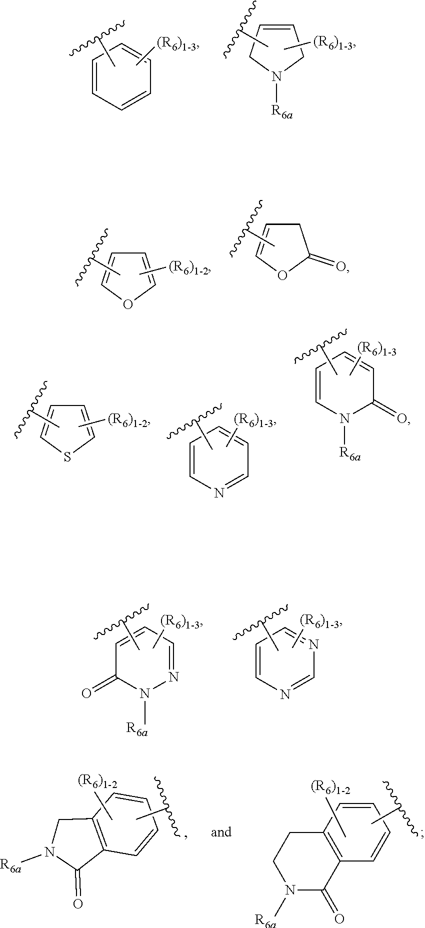

R₄′ is selected from the group consisting of H and C₁₋₃ alkyl;
R₄″ is selected from the group consisting of H, —O(CH₂)₁₋₃OR_{b}, —O(CH₂)₁₋₃NRₐRₐ,

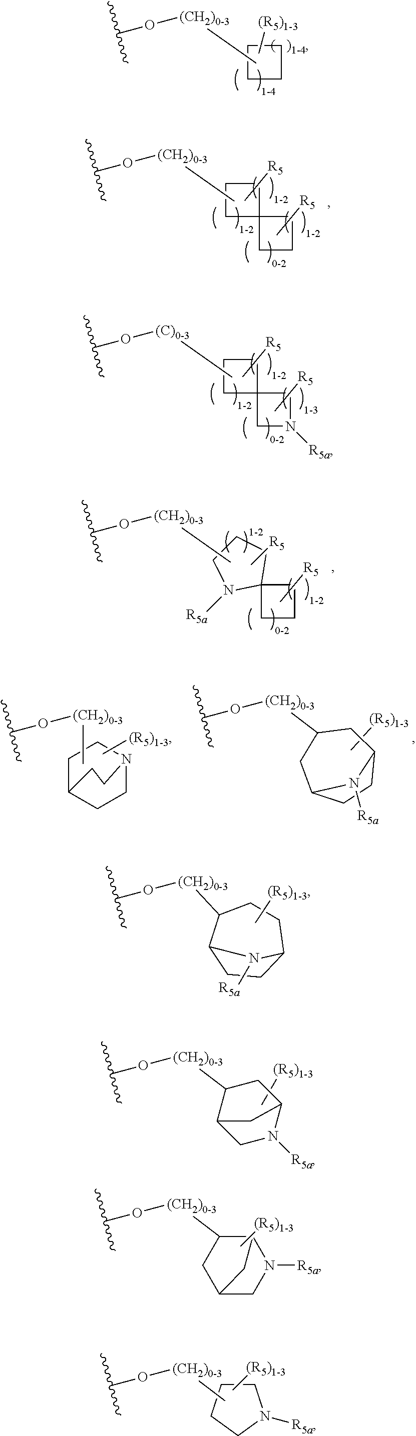

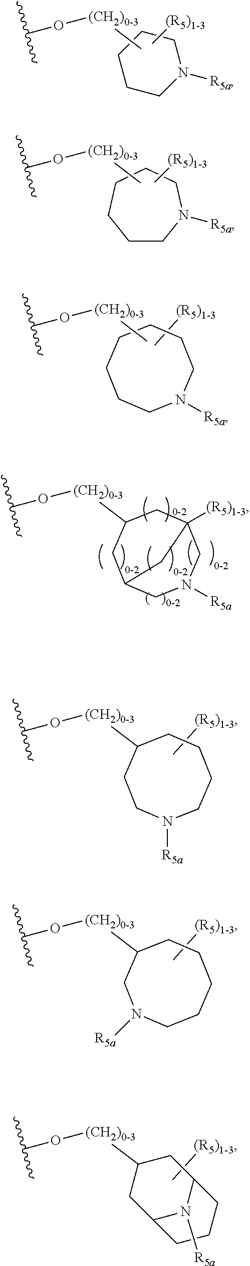

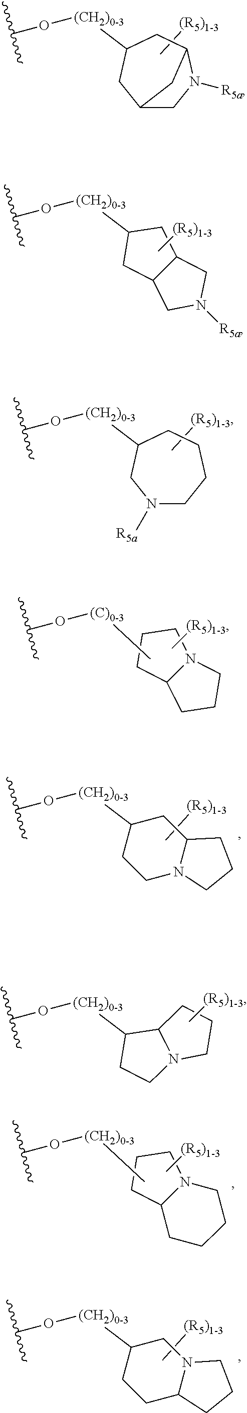

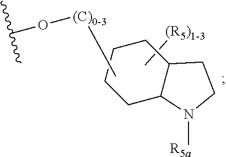

R₅ is independently selected from the group consisting of H, F, Cl, Br, CN, OR_{b}, ═O, —(CH₂)₀₋₁NRₐRₐ, C₁₋₃ alkyl substituted with 1-5 Rₑ, C₃₋₆ carbocyclyl with 1-5 Rₑ, 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₁₋₆ alkyl substituted with 1-5 Rₑ;
R₆ is independently selected from the group consisting of H, F, Cl, Br, CN, OR_{b}, C(═O)R_{b}, —C(═O)NRₐRₐ, —S(═O)₂R_{c}, C₁₋₃ alkyl substituted with 1-5 Rₑ, and heterocyclyl selected from the group consisting of

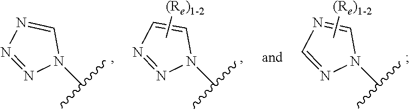

R₆ₐ is independently selected from the group consisting of —C(═O)R_{b}, —C(═O)OR_{b}, —S(═O)ₚR_{c}, —(CH₂)C₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
Rₐ is independently selected from the group consisting of H, C₁₋₃ alkyl, and C₂₋₃ alkynyl;
R_{b} is independently selected from the group consisting of H, C₁₋₃ alky, C₃₋₆ carbocyclyl substituted with 1-3 Rₑ, and 5- to 6-membered heterocyclyl;
Rₑ is independently selected from the group consisting of H, F, Cl, CN, C₁₋₃ alkyl, NR_{f}R_{f}, and —OR_{f}; and
R_{f} is independently selected from the group consisting of H and C₁₋₃ alkyl.

In a nineteenth aspect within the scope of the seventeenth aspect, the present invention provides compounds of Formula (VII):

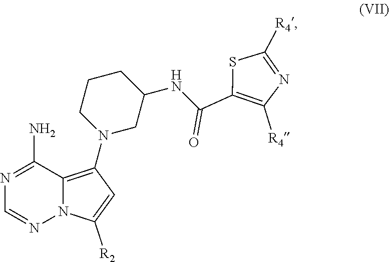

or stereoisomers, tautomers, pharmaceutically acceptable salts or solvates thereof,
wherein:
R₂ is independently selected from the group consisting of

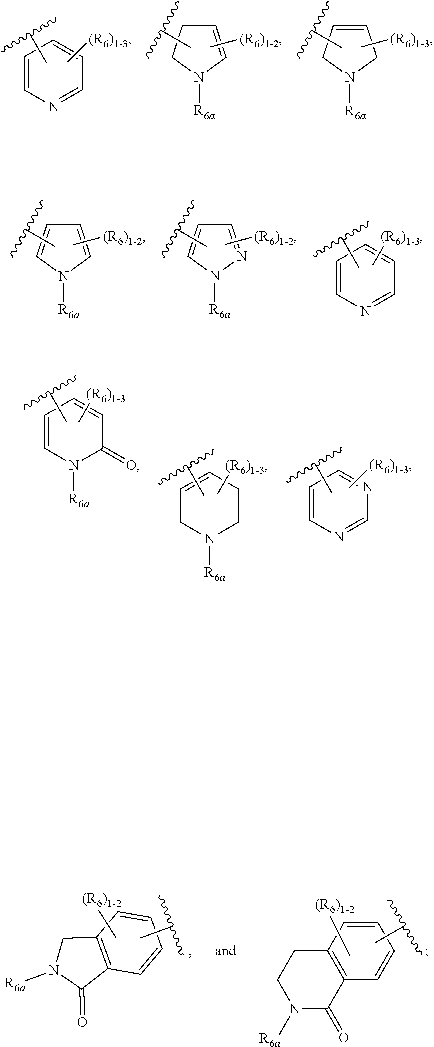

R₄′ is independently selected from the group consisting of H, F, Cl, CN, and C₁₋₃ alkyl substituted with 1-3 Rₑ;
R₄″ is independently selected from the group consisting of H, F, C₁₋₃ alkyl substituted with 1-5 Rₑ, —O(CH₂)₁₋₃OR_{b}, —O(CH₂)₁₋₃C(═O)NRₐRₐ, —O(CH₂)₁₋₃NRₐRₐ, and —O—(CH₂)₀₋₂-4- to 10-membered heterocyclyl comprising carbon atoms and 1-4 heteroatoms selected from N, NR₅ₐ, O, and S(═O)ₚ, and substituted with 1-5 R₅;
R₅ is independently selected from the group consisting of H, F, Cl, Br, CN, OR_{b}, and C₁₋₃ alkyl substituted with 1-5 Rₑ;
R₅ₐ is independently selected from the group consisting of H and C₁₋₃ alkyl substituted with 1-4 Rₑ;
R₆ is independently selected from the group consisting of H, F, Cl, Br, CN, OR_{b}, C₁₋₃ alkyl substituted with 1-5 Re, —C(═O)NRₐRₐ, —S(═O)₂Rₑ, and —(CH₂)ᵣ-5- to 6-membered heterocyclyl comprising carbon atoms and 1-4 heteroatoms selected from the group consisting of N, NR7a, O, and S(═O)p, and substituted with 1-4 R7;
R₆ₐ is independently selected from the group consisting of —C(═O)Rb, —C(═O)ORb, —S(═O)pRc, C1-4 alkyl substituted with 1-5 Re;
Rₐ is independently selected from the group consisting of H and C₁₋₃ alkyl substituted with 1-4 Rₑ;
R_{b} is independently selected from the group consisting of H and C₁₋₃ alkyl substituted with 1-5 Rₑ;
Rₑ is C₁₋₃ alkyl;
Rₑ is independently selected from the group consisting of H, F, Cl, Br, CN, ═O, C₁₋₅ alkyl substituted with 1-4 R_{g}, —OR_{f}, NR_{f}R_{f}, C(═O)R_{f}, and —C(═O)OR_{f};
R_{f} is independently selected from the group consisting of H and C₁₋₅ alkyl;
R_{g} is independently selected from the group consisting of H, F, Cl, Br, OH, C₁₋₅ alkyl, and C3-6 cycloalkyl;
p is an integer independently selected from the group consisting of zero, 1, and 2; and
r is an integer independently selected from the group consisting of zero, 1, 2, 3, and 4.

In a twentieth aspect within the scope of the nineteenth aspect, the present invention provides compounds of Formula (VII), or stereoisomers, tautomers, pharmaceutically acceptable salts or solvates thereof, wherein:
R₂ is independently selected from the group consisting of

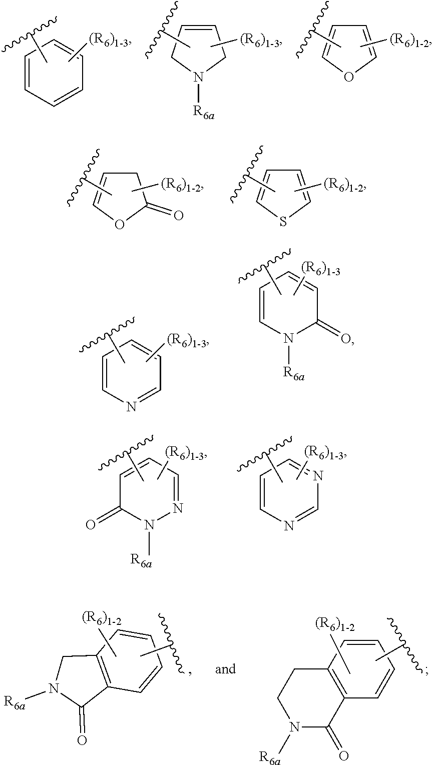

R₄′ is C₁₋₃ alkyl;
R₄″ is independently selected from the group consisting of —O(CH₂)₁₋₃OR_{b}, —O(CH₂)₁₋₃NRₐRₐ,

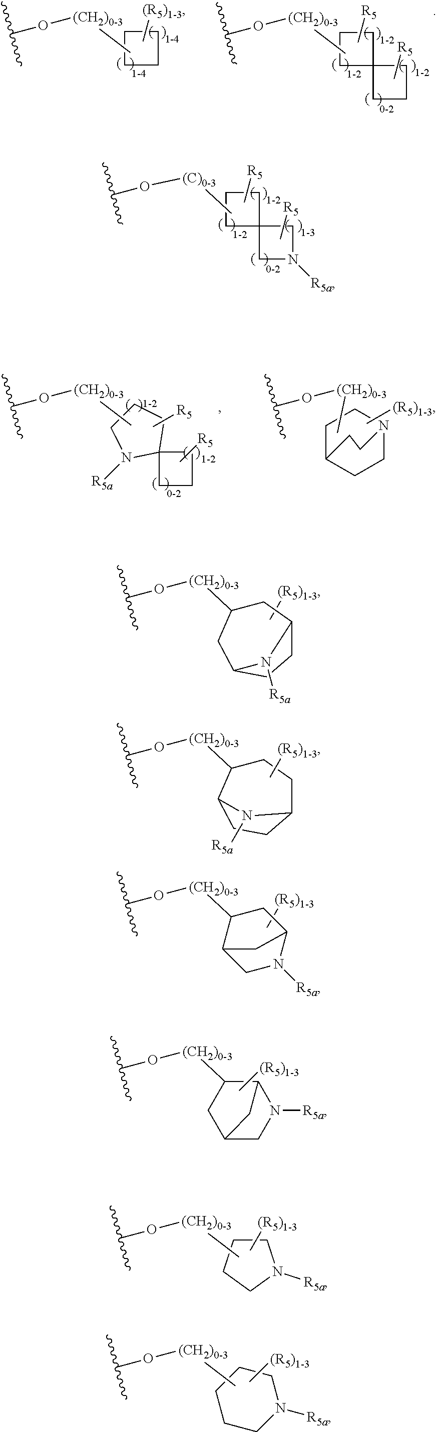

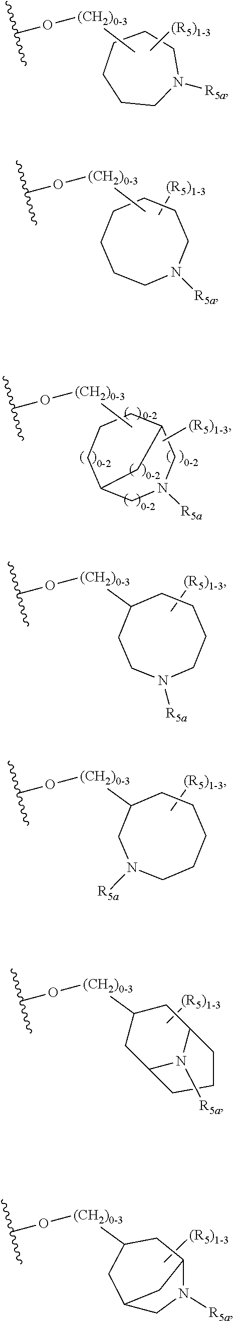

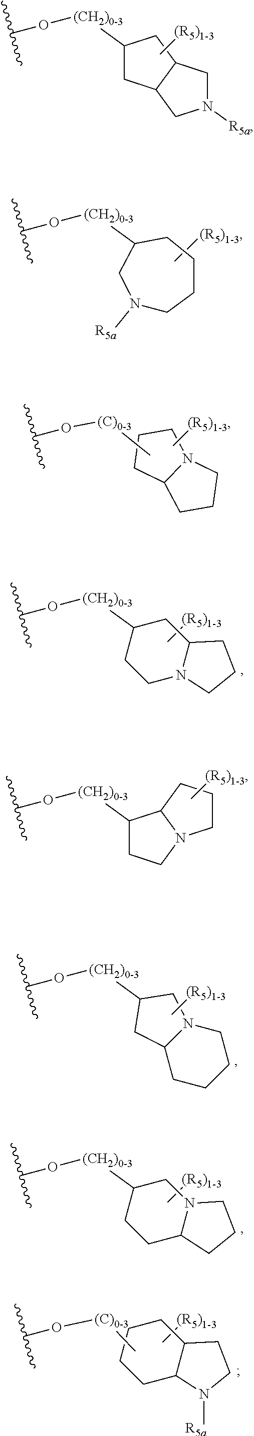

R₅ is independently selected from the group consisting of H, F, Cl, Br, CN, OR_{b}, ═O, —(CH₂)₀₋₁NRₐRₐ, C₁₋₃ alkyl substituted with 1-5 Rₑ, C₃₋₆ carbocyclyl with 1-5 Rₑ, 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₁₋₆ alkyl substituted with 1-5 Rₑ;
R₆ is independently selected from the group consisting of H, F, Cl, Br, CN, OR_{b}, C(═O)R_{b}, —C(═O)NRₐRₐ, —S(═O)₂R_{c}, C₁₋₃ alkyl substituted with 1-5 Rₑ, and heterocyclyl selected from the group consisting of

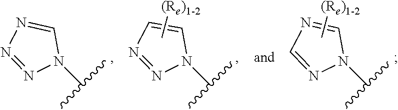

R₆ₐ is independently selected from the group consisting of —C(═O)R_{b}, —C(═O)OR_{b}, —S(═O)ₚR_{c}, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
Rₐ is independently selected from the group consisting of H, C₁₋₃ alkyl, and C₂₋₃ alkynyl;
R_{b} is independently selected from the group consisting of H, C₁₋₃ alky, C₃₋₆ carbocyclyl substituted with 1-3 Rₑ, and 5- to 6-membered heterocyclyl;
Rₑ is independently selected from the group consisting of H, F, Cl, CN, C₁₋₃ alkyl, NR_{f}R_{f}, and —OR_{f}; and
R_{f} is independently selected from the group consisting of H and C₁₋₃ alkyl.

In a twenty first aspect within the scope of the second aspect, the present invention provides compounds of Formula (VIII):

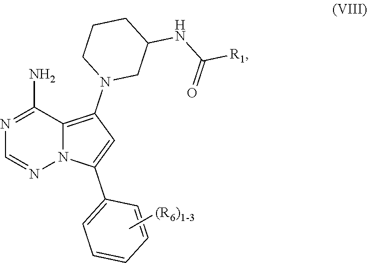

or stereoisomers, tautomers, pharmaceutically acceptable salts or solvates thereof,
wherein:
R₁ is independently selected from the group consisting of

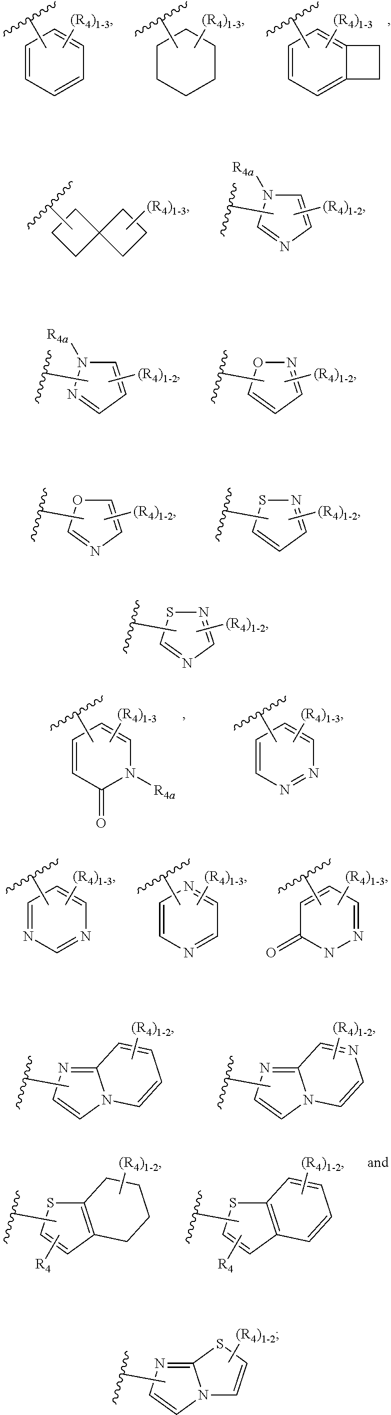

R₄ is independently selected from the group consisting of H, F, Cl, Br, ═O, CN, —OR_{b}, C₁₋₄ alkyl substituted with 1-5 Rₑ, C₂₋₄ alkenyl substituted with 1-5 Rₑ, C₂₋₄ alkynyl substituted with 1-5 Rₑ; C₃₋₆ carbocyclyl substituted with 1-5 R₅, 5- to 7-membered heterocyclyl comprising carbon atoms and 1-4 heteroatoms selected from N, NR₅ₐ, O, and S(═O)ₚ, and substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ is independently selected from the group consisting of H, C₁₋₄ alkyl substituted with 1-5 Rₑ;
R₅ is independently selected from the group consisting of H, F, Cl, Br, CN, OR_{b}, ═O, —NRₐRₐ, C₁₋₅ alkyl substituted with 1-5 Rₑ, C₃₋₆ carbocyclyl with 1-5 Rₑ, and 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₁₋₆ alkyl substituted with 1-5 Rₑ;
R₆ is independently selected from the group consisting of H, F, Cl, OR_{b}, —S(═O)₂R_{c}, and 5- to 6-membered heterocyclyl comprising carbon atoms and 1-4 heteroatoms selected from the group consisting of N, NR₇ₐ, O, and S(═O)ₚ, and substituted with 1-5 R₇;
Rₐ is independently selected from the group consisting of H and C₁₋₃ alkyl;
R_{b} is independently selected from the group consisting of H and C₁₋₃ alkyl;
R_{c} is independently selected from the group consisting of C₁₋₃ alkyl and cycloalkyl; and
Rₑ is independently selected from the group consisting of H, C₁₋₄ alkyl F, Cl, and Br.

In a twenty second aspect within the scope of the second aspect, the present invention provides compounds of Formula (II), or stereoisomers, tautomers, pharmaceutically acceptable salts or solvates thereof, wherein:
R₁ is independently selected from the group consisting of

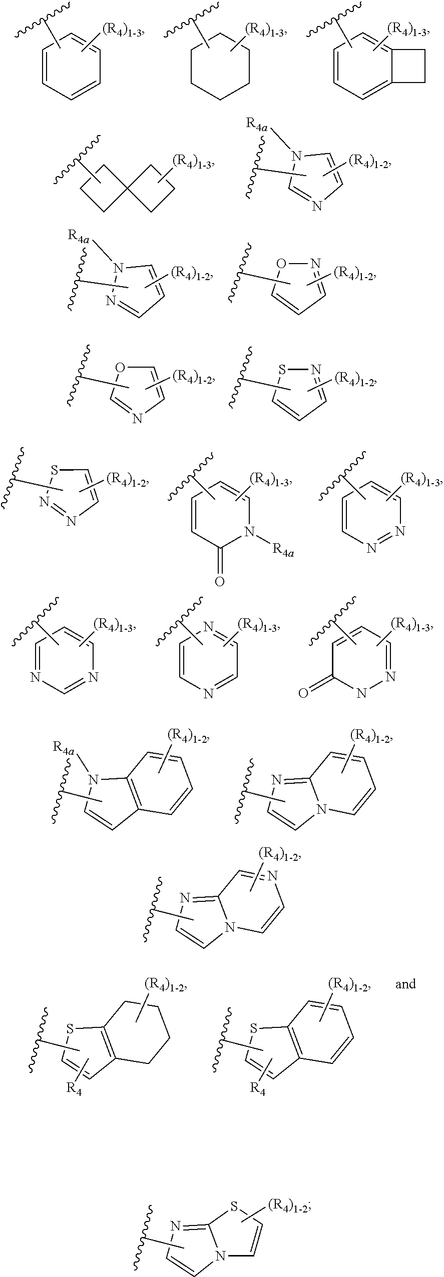

R₂ is independently selected from the group consisting of

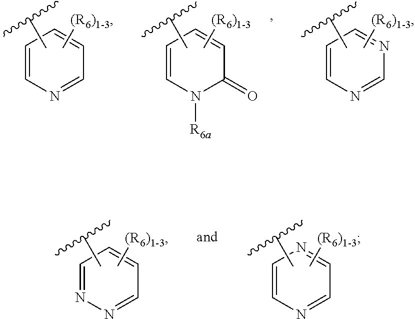

R₄ is independently selected from the group consisting of H, F, Cl, Br, ═O, CN, —OR_{b}, C₁₋₄ alkyl substituted with 1-5 Rₑ, C₂₋₄ alkenyl substituted with 1-5 Rₑ, C₂₋₄ alkynyl substituted with 1-5 Rₑ; C₃₋₆ carbocyclyl substituted with 1-5 R₅, 5- to 7-membered heterocyclyl comprising carbon atoms and 1-4 heteroatoms selected from N, NR₅ₐ, O, and S(═O)ₚ, and substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ is independently selected from the group consisting of H and C₁₋₄ alkyl substituted with 1-5 Rₑ;
R₅ is independently selected from the group consisting of H, F, Cl, Br, CN, OR_{b}, ═O, —NRₐRₐ, C₁₋₅ alkyl substituted with 1-5 Rₑ, C₃₋₆ carbocyclyl with 1-5 Rₑ, and 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₃₋₆ alkyl substituted with 1-5 Rₑ;
R₆ is independently selected from the group consisting H, F, Cl, Br, CN, —(CH₂)ᵣNRₐRₐ, —OR_{b}, —C(═O)NRₐRₐ, —C(═O)NRₐ(CH₂)ᵣOR_{b}, —C(═O)R_{b}, —C(═O)(CH₂)OR_{b}, —NRₐC(═O)R_{b}, —NRₐC(═O)OR_{b}, —NRₐC(═O)(CH₂)ᵣNRₐRₐ, —S(═O)_{P}R_{c}, and —NRₐS(═O)_{P}R_{c}, C₁₋₄ alkyl substituted with 1-5 Rₑ, and 5- to 6-membered heterocyclyl comprising carbon atoms and 1-4 heteroatoms selected from the group consisting of N, NR₇ₐ, O, and S(═O)ₚ, and substituted with 1-5 R₇;
R₆ₐ is independently selected from the group consisting of H and C₁₋₅ alkyl substituted with 1-5 Rₑ;
Rₐ is independently selected from the group consisting of H and C₁₋₃ alkyl;
R_{b} is independently selected from the group consisting of H and C₁₋₃ alkyl;
R_{c} is C₁₋₃ alkyl;
Rₑ is independently selected from the group consisting of H, F, Cl, CN, C₁₋₃ alkyl, and —OR_{f}; and
R_{f} is independently selected from the group consisting of H and C₁₋₃ alkyl.

In a twenty third aspect within the scope of the second aspect, the present invention provides compounds of Formula (IX):

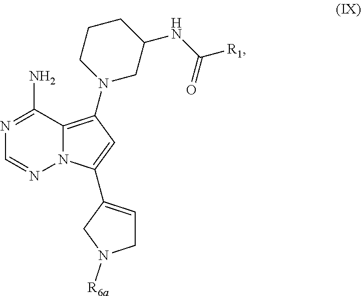

or stereoisomers, tautomers, pharmaceutically acceptable salts or solvates thereof,
wherein:
R₁ is independently selected from the group consisting of

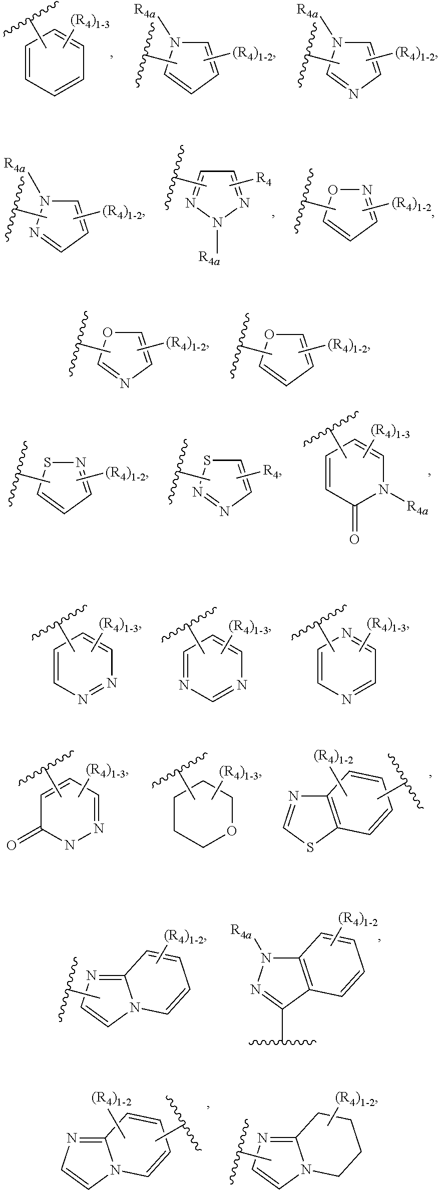

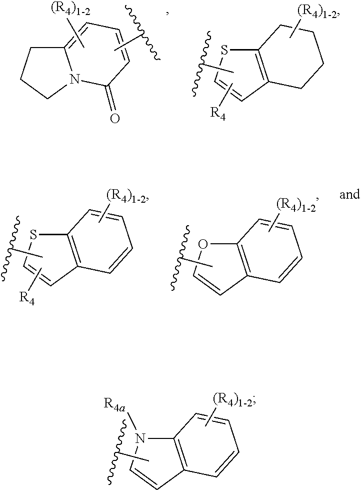

R₄ is independently selected from the group consisting of H, F, Cl, Br, ═O, CN, —NRₐRₐ, —OR_{b}, —O(CH₂)ᵣOR_{b}, —O(CH₂)ᵣC(═O)NRₐRₐ, —O(CH₂)ᵣNRₐC(═O)R_{b}, —O(CH₂)ᵣNRₐC(═O)OR_{b}, —O(CH₂)ᵣNRₐRₐ, C₁₋₄ alkyl substituted with 1-5 Rₑ, C₃₋₆ carbocyclyl substituted with 1-5 R₅, 4- to 10-membered heterocyclyl comprising carbon atoms and 1-4 heteroatoms selected from N, NR₅ₐ, O, and S(═O)ₚ, and substituted with 1-5 R₅; and —O-4- to 10-membered heterocyclyl comprising carbon atoms and 1-4 heteroatoms selected from N, NR₅ₐ, O, and S(═O)ₚ, and substituted with 1-5 R₅;
R₄ₐ is independently selected from the group consisting of H, C₁₋₃ alkyl, C₃₋₆ carbocyclyl, and heterocyclyl comprising carbon atoms and 1-4 heteroatoms selected from N, NR₅ₐ, O, and S(═O)ₚ, and substituted with 1-5 R₅;
R₅ is independently selected from the group consisting of H, F, Cl, Br, CN, OR_{b}, ═O, —NRₐRₐ, C₁₋₅ alkyl substituted with 1-5 Rₑ, C₃₋₆ carbocyclyl with 1-5 Rₑ, and 3-6 membered heterocyclyl comprising 1-4 heteroatoms selected from N, NR_{5b}, O, and S(═O)ₚ, and substituted with 1-5 Rₑ;
R₅ₐ is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b} is independently selected from the group consisting of H and C₁₋₆ alkyl substituted with 1-5 Rₑ;
R₆ₐ is independently selected from the group consisting of —C(═O)NRₐRₐ, —C(═O)R_{b}, —C(═O)(CH₂)₀₋₂OR_{b}, —S(═O)₂R_{c}, —S(═O)ₚNRₐRₐ, and —(CH₂)ᵣ-4- to 9-membered heterocyclyl comprising carbon atoms and 1-4 heteroatoms selected from the group consisting of N, NR₇ₐ, O, and S(═O)ₚ, and substituted with 1-3 R₇;
R₇ is independently selected from the group consisting of H, F, Cl, Br, CN, OR_{b}, ═O, and C₁₋₅ alkyl substituted with 1-5 Rₑ;
R₇ₐ is independently selected from the group consisting of H and C₁₋₅ alkyl substituted with 1-5 Rₑ;
Rₐ is independently selected from the group consisting of H and C₁₋₃ alkyl substituted with 1-3 Rₑ;
R_{b} is independently selected from the group consisting of H, C₁₋₄ alkyl substituted with 1-3 Rₑ, C₃₋₆carbocyclyl substituted with 1-3 Rₑ,
Rₑ is independently selected from the group consisting of H, F, Cl, Br, CN, ═O, C₁₋₃ alkyl substituted with 1-4 R_{g}, and —OR_{f}; and
R_{f} is independently selected from the group consisting of H and C₁₋₃ alkyl.

In a twenty fourth aspect within the scope of the first aspect, the present invention provides compounds of Formula (VII):

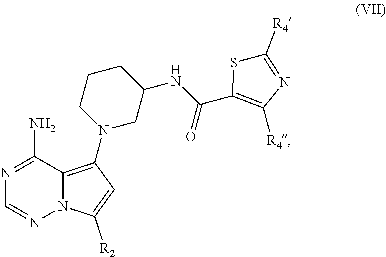

or a pharmaceutically acceptable salt thereof, wherein:
R₂ is selected from the group consisting CN,

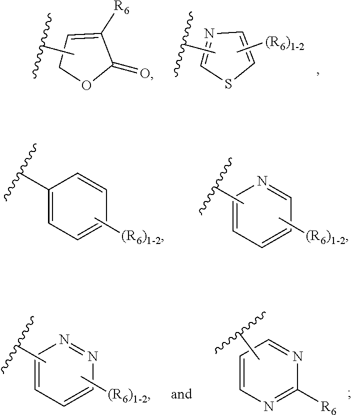

R₄′ is selected from the group consisting of CH₃ and CD₃;
R₄″ is selected from the group consisting of

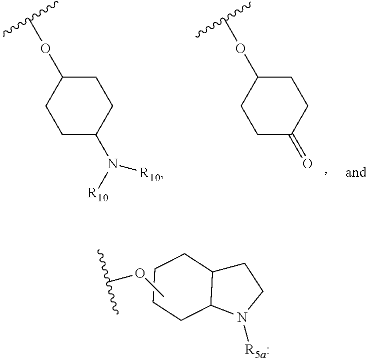

R₅ₐ is selected from the group consisting of H and C₁₋₃ alkyl;
R₆, at each occurrence, is independently selected from the group consisting of H, OR_{b}, —C(═O)OR_{b}, —C(═O)NRₐRₐ, —S(═O)₂R_{c}, and C₁₋₃ alkyl,
R₁₀, at each occurrence, is independently selected from the group consisting of H, CH₃, CD₃, CH₂CH₃,

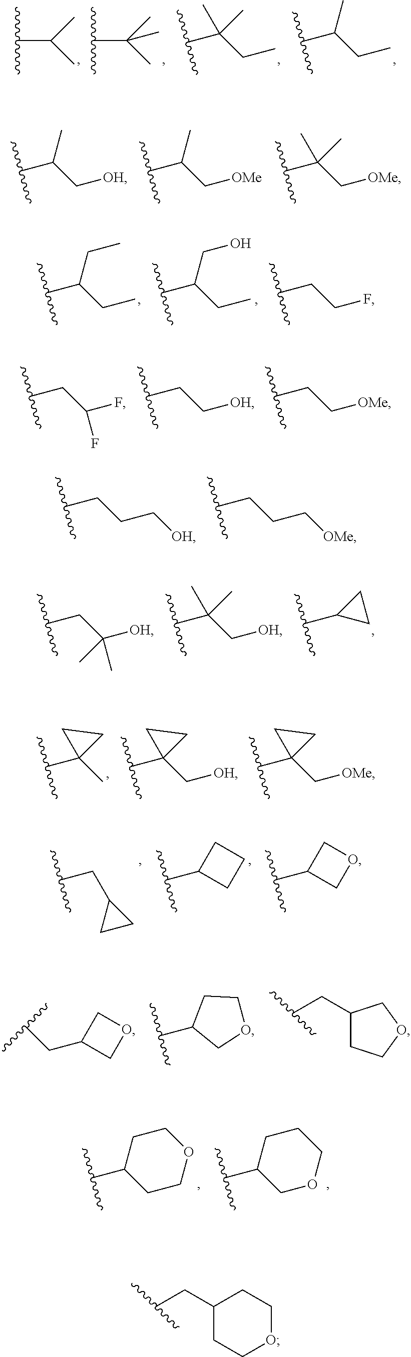

or R₁₀ and R₁₀ together with the nitrogen atom to which they are both attached form a heterocyclyl selected from the group consisting of

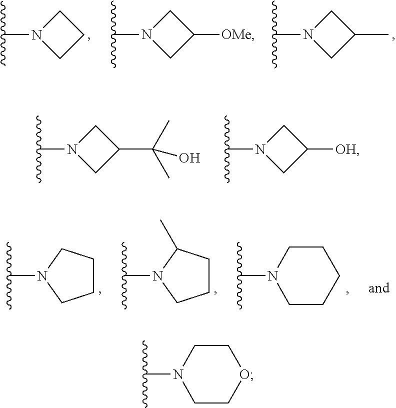

and
R_{b} is selected from the group consisting of CH₃ and CD₃.

In a twenty fifth aspect within the scope of the twenty fourth aspect, the present invention provides compounds of Formula (X):

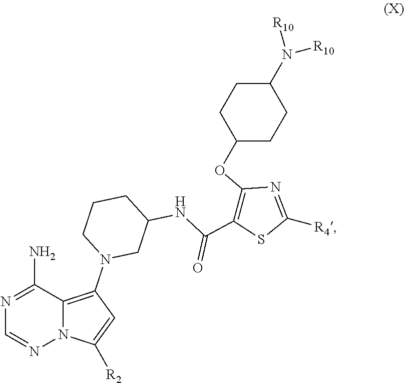

or a pharmaceutically acceptable salt thereof, wherein:
R₂ is selected from the group consisting of

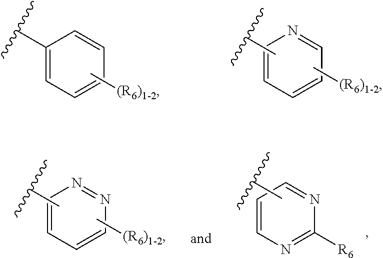

R₄′ is selected from the group consisting of CH₃ and CD₃;
R₆, at each occurrence, is independently selected from the group consisting of OR_{b}, C(═O)R_{b}, —C(═O)NRₐRₐ, —S(═O)₂R_{c}, C₁₋₃ alkyl substituted with 1-5 Rₑ,
R₁₀, at each occurrence, is independently selected from the group consisting of H, CH₃, CD₃, CH₂CH₃,

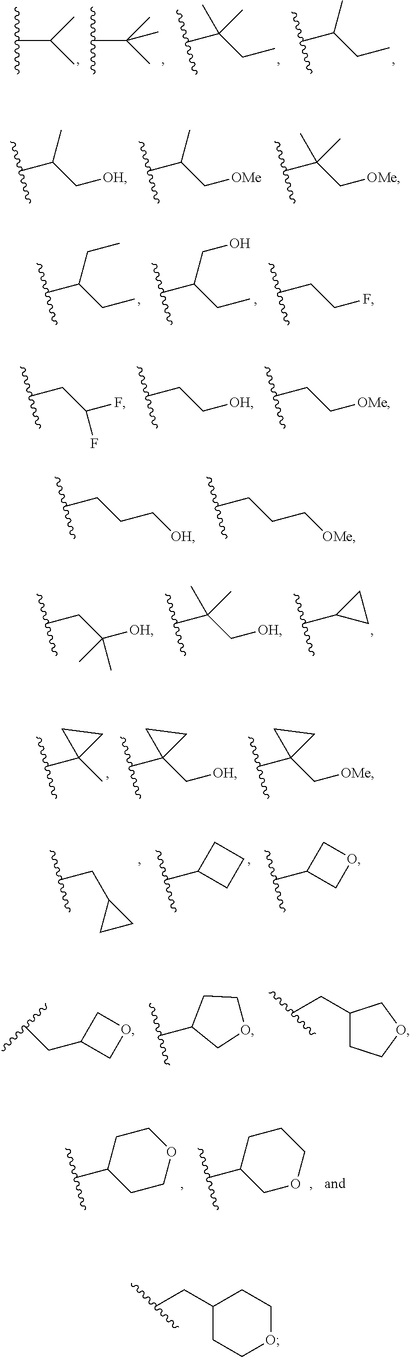

or R₁₀ and R₁₀ together with the nitrogen atom to which they are both attached form a heterocyclyl selected from the group consisting of

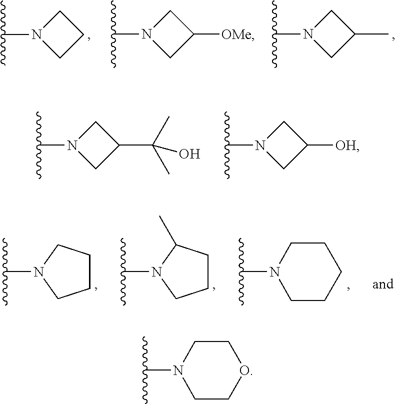

In a twenty sixth aspect within the scope of the twenty fifth aspect, the present invention provides compounds of Formula (X), or a pharmaceutically acceptable salt thereof, wherein:
R₂ is selected from the group consisting of

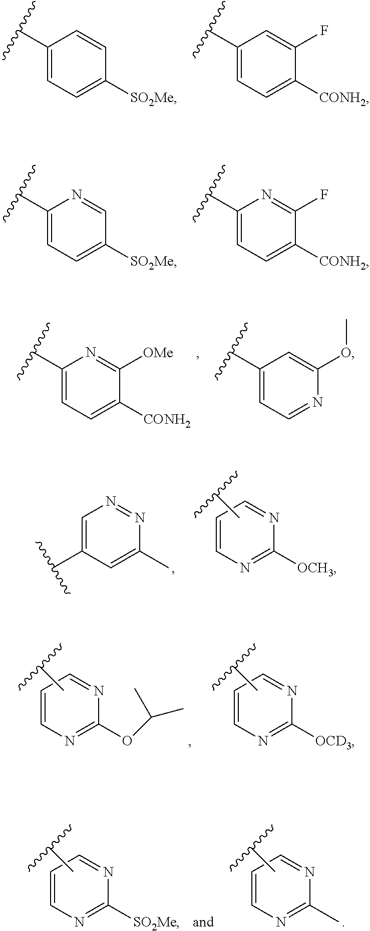

In a twenty seventh aspect within the scope of the twenty fourth aspect, the present invention provides compounds of Formula (X):

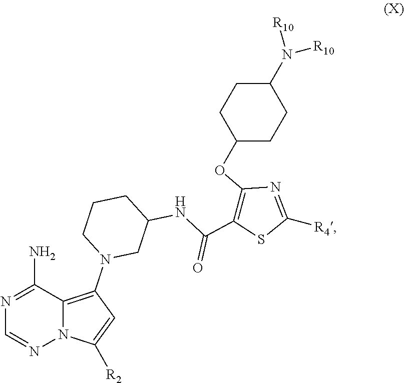

or a pharmaceutically acceptable salt thereof, wherein:
R₂ is selected from the group consisting of

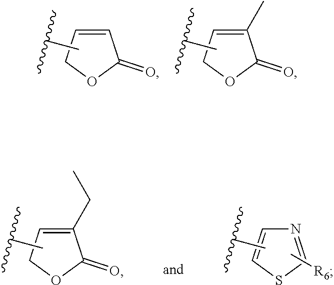

R₄′ is selected from the group consisting of CH₃ and CD₃;
R₆ is selected from the group consisting of —C(═O)NH₂, —C(═O)NHCH₃, and —C(═O)OH;
R₁₀, at each occurrence, is independently selected from the group consisting of H, CH₃, CD₃, CH₂CH₃,

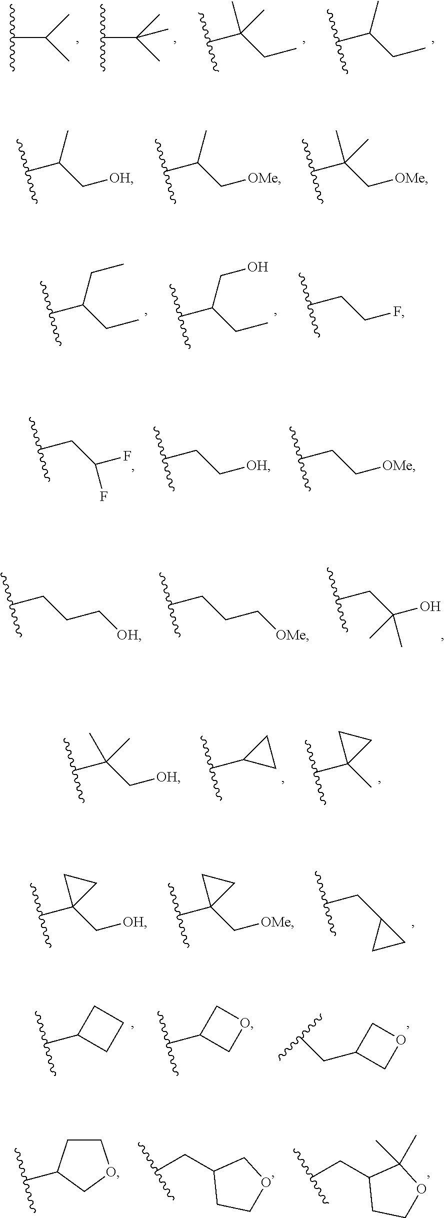

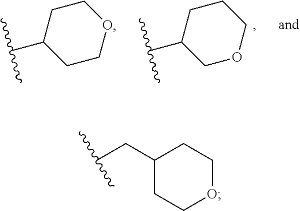 [SMILES: *C1CCOCC1 ; *C1CCCOC1 ; *CC1CCOCC1]

or R₁₀ and R₁₀ together with the nitrogen atom to which they are both attached form a heterocyclyl selected from the group consisting of

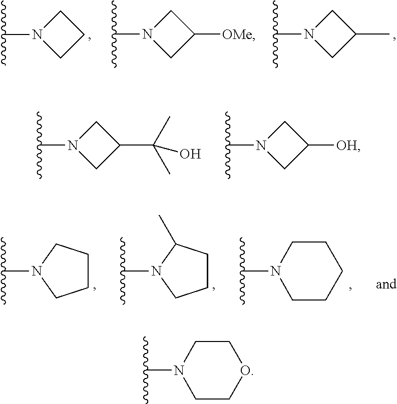

In one embodiment of the compounds of Formula (I) or (II), R₁ is

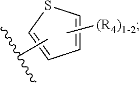

### R₂ is

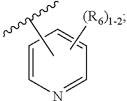

R₄ is H, F, Cl, CH₃CHF₂, —O(CH₂)₁₋₃N(CH₃)₂, C₃₋₆ cycloalkyl,

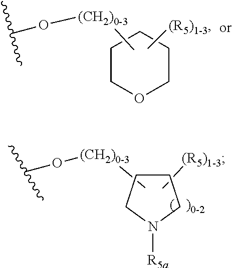

R₅ is H; R₅ₐ is H or C₁₋₃ alkyl substituted with 0-2 F, Cl, or OH; and R₆ is H, F, Cl, CH₃, CHF₂, CN,

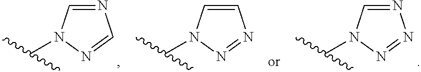 [SMILES: *n1cncn1 ; *n1ccnn1 ; *n1cnnn1]

In another embodiment of the compounds of Formula (I) or (II), R₁ is

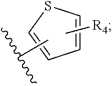

### R₂ is

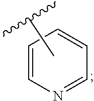

R₄ is CH₃.

In another embodiment of the compounds of Formula (I) or (II), R₁ is

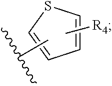

### R₂ is

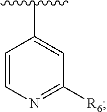

R₄ is CH₃; and R₆ is F, Cl, CH₃, or CN.

In another embodiment of the compounds of Formula (I) or (II), R₂ is

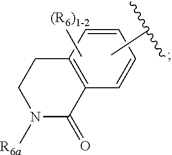

R₆ is H, F, Cl, CH₃, or CN; R₆ₐ is H or C₁₋₃ alkyl.

In another embodiment of the compounds of Formula (I) or (II), R₂ is

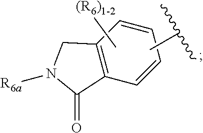

R₆ is H, F, Cl, CH₃, or CN; R₆ₐ is H or C₁₋₃ alkyl.

In another embodiment of the compounds of Formula (I) or (II), R₁ is

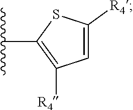

### R₂ is

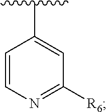

R₄′ is F, Cl, or CH₃; R₄″ is —O(CH₂)₁₋₃N(CH₃)₂,

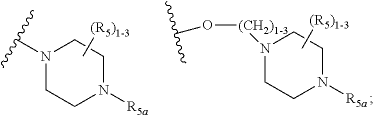

R₅ is H; R₅ₐ is CH₃; and R₆ is F, Cl, CH₃, CHF₂, or CN.

In another embodiment of the compounds of Formula (I) or (II), R₁ is

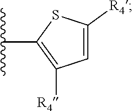

### R₂ is

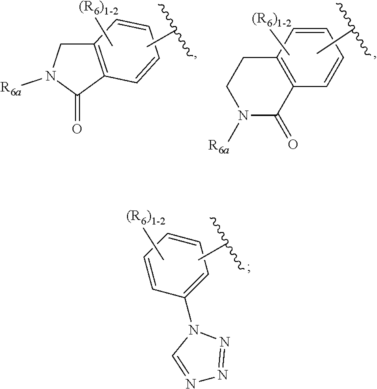

R₄′ is F, Cl, or CH₃; R₄″ is H, —O(CH₂)₁₋₃N(CH₃)₂,

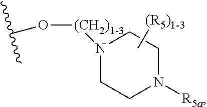

of

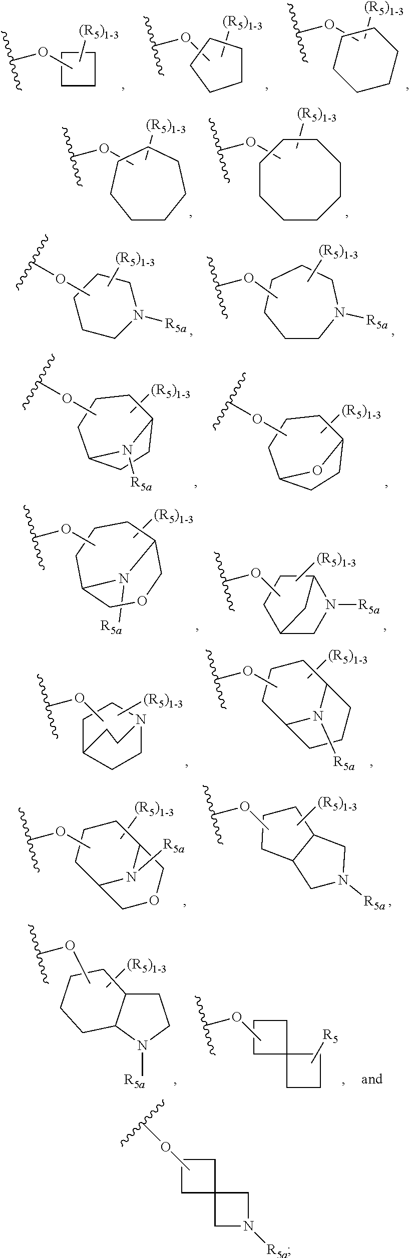

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R₆ is H, F, CH₃, OCH₃, OCH₂CH₃, OCH₂CHF₂, OCH₂CH₂OH, CN, CHF₂, C(═O)CH₃, or C(═O)NH₂; R₆ₐ is H, C₁₋₃ alkyl, C(═O)CH₃, and C(═O)OCH₃.

In another embodiment of the compounds of Formula (I) or (II), R₁ is

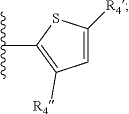

### R₂ is

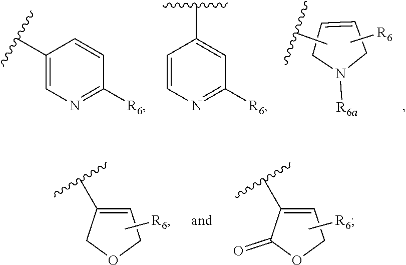

R₄′ is F, Cl, or CH₃; R₄″ is H, —O(CH₂)₁₋₃N(CH₃)₂,

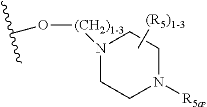

of

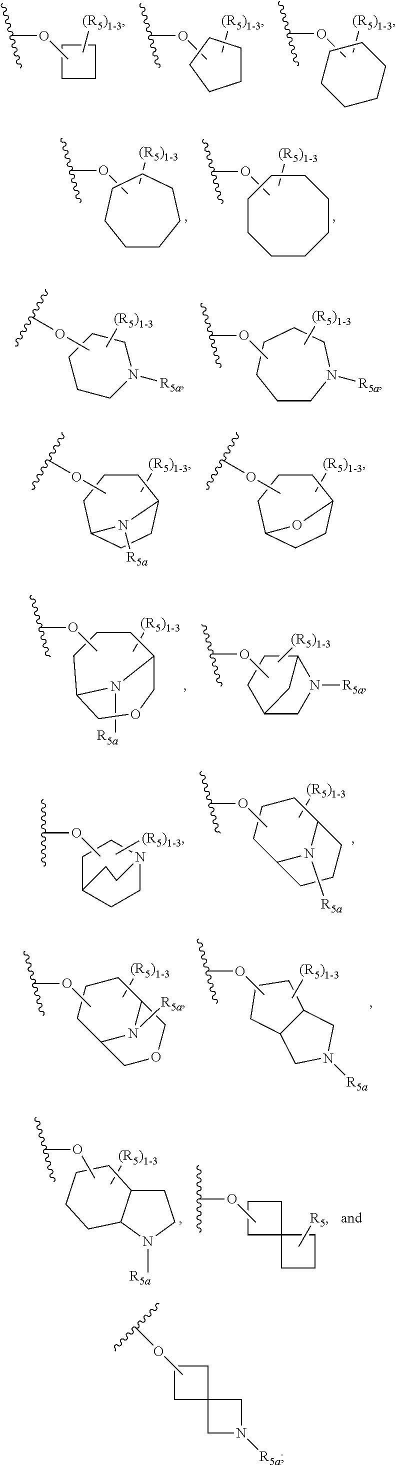

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R₆ is H, F, CH₃, OCH₃, OCH₂CH₃, OCH₂CHF₂, OCH₂CH₂OH, CN, CHF₂, C(═O)CH₃, or C(═O)NH₂; R₆ₐ is C(═O)CH₃, C(═O)OCH₃.

In another embodiment of the compounds of Formula (I) or (II), R₁ is

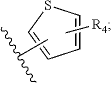

### R₂ is

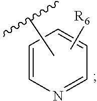

R₄ is H, F, or Cl; and R₆ is

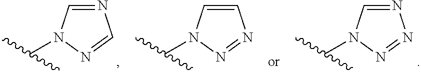 [SMILES: *n1cncn1 ; *n1ccnn1 ; *n1cnnn1]

In one embodiment of the compounds of Formula (I) or (II), R₁ is

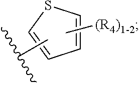

### R₂ is

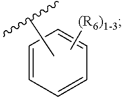

R₄ is H, F, Cl, CHF₂, —O(CH₂)₁₋₃N(CH₃)₂, C₃₋₆ cycloalkyl,

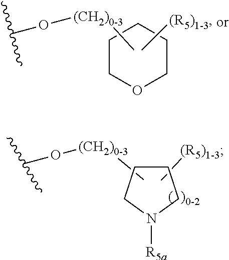

R₅ is H or C₁₋₃ alkyl; R₅ₐ is H, CH₃, Et, i-Pr, CH₂CH₂OH, CHMeCH₂OH, CH₂CMe₂OH, C(═O)CH₃, C(═O)OCH₃; and R₆ is H, F, Cl, Br, CN, C(═O)H, C(═O)NH₂,

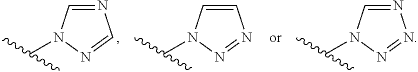 [SMILES: *n1cncn1 ; *n1ccnn1 ; *n1cnnn1]

In another embodiment of the compounds of Formula (I) or (II), R₁ is

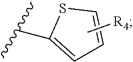

### R₂ is

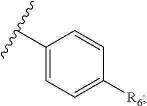

R₄ is F, Cl, or CH₃; and R₆ is —S(═O)₂CH₃.

In another embodiment of the compounds of Formula (I) or (II), R₁ is

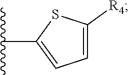

### R₂ is

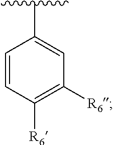

R₄ is H, F, Cl, CH₃; R₆″ is H, F, Cl; and R₆′ is CN, C(═O)NH₂,

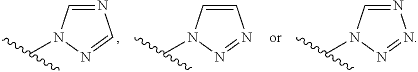 [SMILES: *n1cncn1 ; *n1ccnn1 ; *n1cnnn1]

In another embodiment of the compounds of Formula (I) or (II), R₁ is

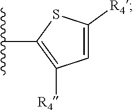

### R₂ is

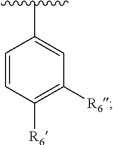

R₄′ is H, F, Cl, CH₃CHF₂, or cyclopropyl; R₄′ is H, —O(CH₂)₁₋₃N(CH₃)₂,

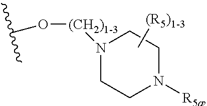

of

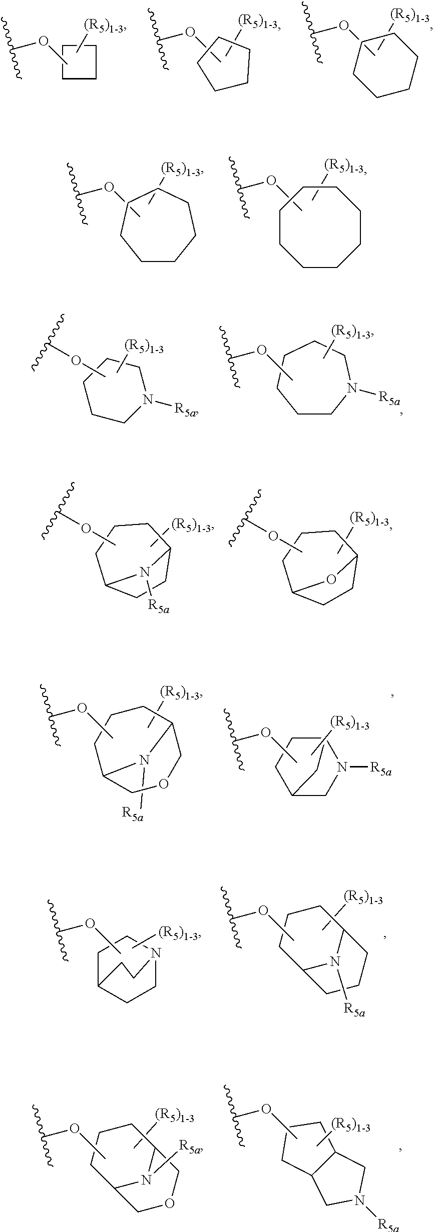

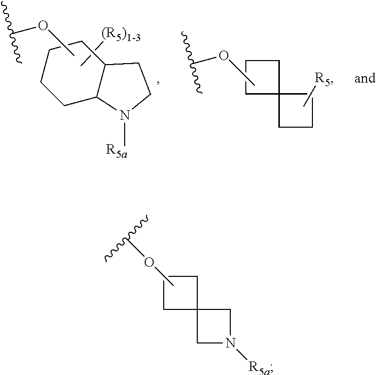

R₅ is H; R₅ₐ is H, CH₃, Et, i-Pr, CH₂CH₂OH, CHMeCH₂OH, CH₂CMe₂OH, C(═O)CH₃, C(═O)OCH₃; R₆″ is H, F, Cl; and R₆′ is C(═O)H, C(═O)NH₂,

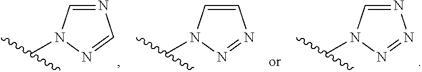 [SMILES: *n1cncn1 ; *n1ccnn1 ; *n1cnnn1]

In another embodiment of the compounds of Formula (I) or (II), R₁ is

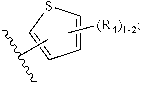

### R₂ is

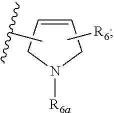

R₄ is H, F, Cl, CH₃,

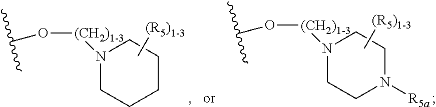

R₅ is H; R₅ₐ is H or CH₃; R₅ₐ is H, CH₃, Et, i-Pr, CH₂CH₂OH, CHMeCH₂OH, CH₂CMe₂OH, C(═O)CH₃, C(═O)OCH₃; and R₆ is H; R₆ₐ is —C(═O)CH₃, —C(═O)OCH₃, or —S(═O)₂CH₃.

In another embodiment of the compounds of Formula (I) or (II), R₁ is

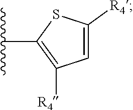

### R₂ is

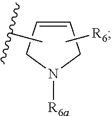

R₄′ is H, F, Cl, CH₃, CHF₂; R₄″ is H, —O(CH₂)₁₋₃N(CH₃)₂,

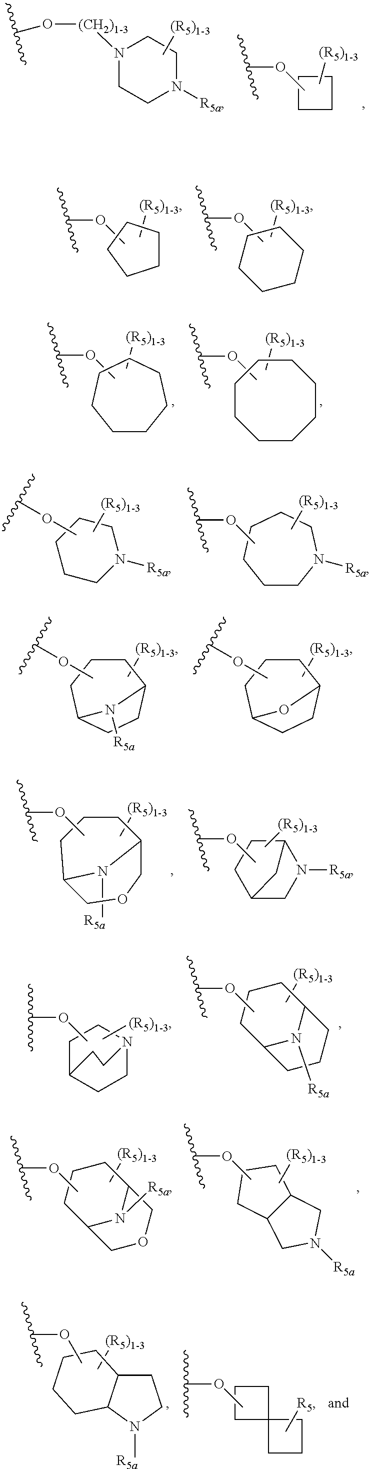

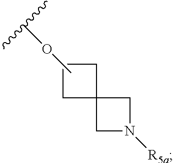

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R₆ is H; and R₆ₐ is —C(═O)CH₃, —C(═O)OCH₃, or —S(═O)₂CH₃.

In another embodiment of the compounds of Formula (I) or (II), R₁ is

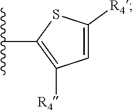

### R₂ is

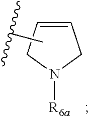

R₄′ is F, Cl, or CH₃; R₄″ is H, —O(CH₂)₁₋₃N(CH₂)₂,

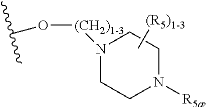

of

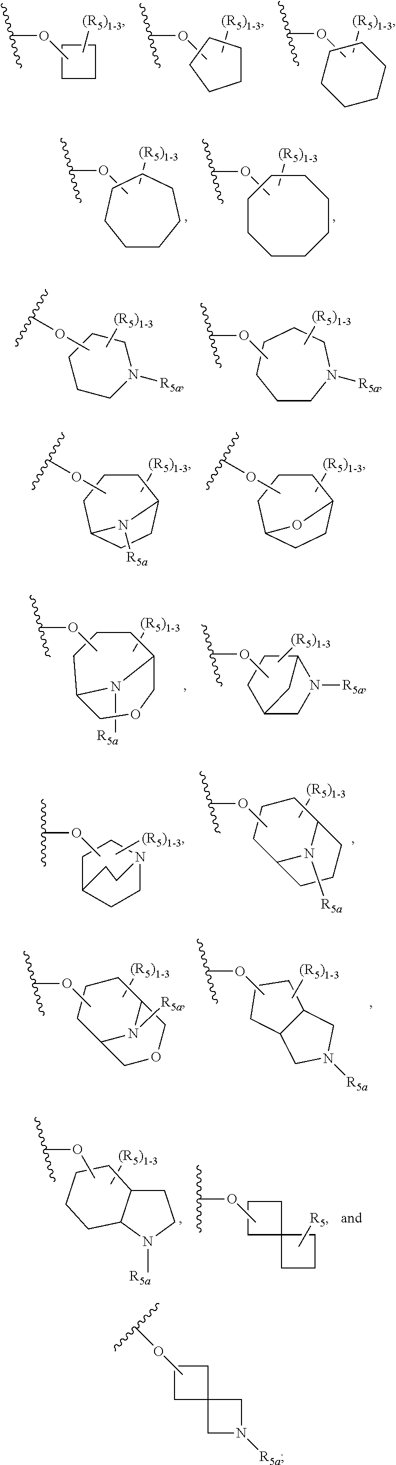

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R₆ₐ is —C(═O)R_{b}; R_{b} is CH₃,

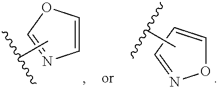

In another embodiment of the compounds of Formula (I) or (II), R₁ is

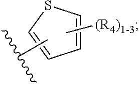

### R₂ is

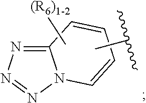

R₄ is H, F, Cl, CH₃; and R₆ is H.

In one embodiment of the compounds of Formula (I) or (II), R₁ is

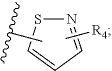

### R₂ is

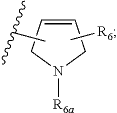

R₄ is H, F, Cl, or OCH₃; and R₆ is H; R₆ₐ is —S(═O)₂CH₃ or C(═O)CH₃.

In one embodiment of the compounds of Formula (I) or (II), R₁ is

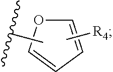

### R₂ is

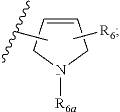

R₄ is phenyl; and R is H; R₆ₐ is —S(═O)₂CH₃ or C(═O)CH₃.

In one embodiment of the compounds of Formula (I) or (II), R₁ is

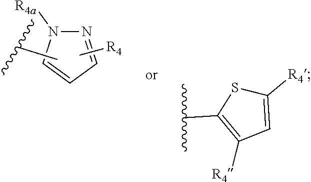

### R₂ is

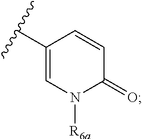

R₄′ is H, F, Cl, CH₃, CHF₂, or phenyl; R₄″ is H or

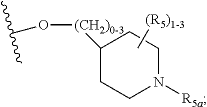

R₅ is H; R₅ₐ is H or CH₃; and R₆ₐ is CHF₂ or CH₂CHF₂.

In one embodiment of the compounds of Formula (I) or (II), R₁ is

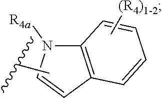

### R₂ is

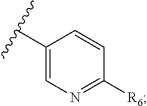

R₄ is F, Cl, CH₃, —O(CH₂)₁₋₃N(CH₃)₂, or

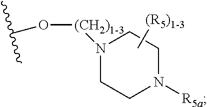

R₄ₐ is H; R₅ is H; and R₆ is CN or CHF₂.

In one embodiment of the compounds of Formula (I) or (II), R₁ is

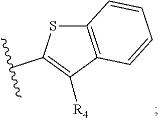

### R₂ is

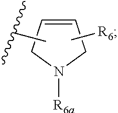

R₄ is H or —O(CH₂)₁₋₃N(CH₃)₂, or

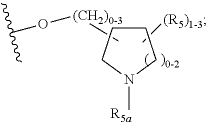

R₅ is H; R₅ₐ is H or CH₃; and R₆ is H; R₆ₐ is —C(═O)CH₃, —C(═O)OCH₃, or —S(═O)₂CH₃.

In one embodiment of the compounds of Formula (I) or (II), R₁ is

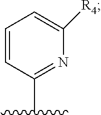

### R₂ is

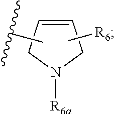

### R₄ is

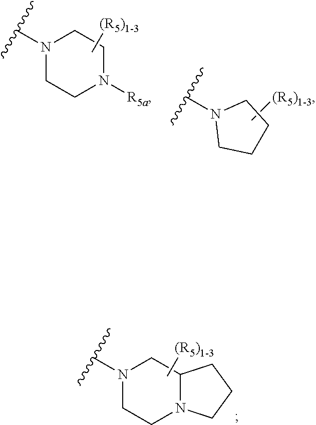

R₅ is H or CH₃; R₅ₐ is H or CH₃; R₆ is H; R₆ₐ is —C(═O)CH₃, —C(═O)OCH₃, or —S(═O)₂CH₃.

In one embodiment of the compounds of Formula (I) or (II), R₁ is

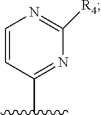

### R₂ is

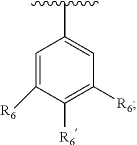

### R₄ is H, or

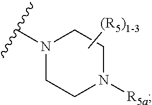

R₅ s is H; R₅ₐ is H or CH₃; R₆ is H, F, Cl; and R₆′ is C(═O)H, C(═O)NH₂,

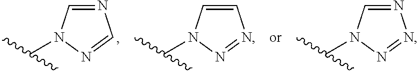 [SMILES: *n1cncn1 ; *n1ccnn1 ; *n1cnnn1]

In another embodiment of the compounds of Formula (I) or (II), R₁ is

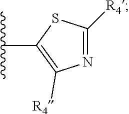

### R₂ is

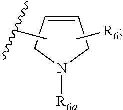

R₄′ is H, CH₃, CHF₂, Et, iPr, or c-Pr; R₄″ is H, —O(CH₂)₁₋₃N(CH₃)₂,

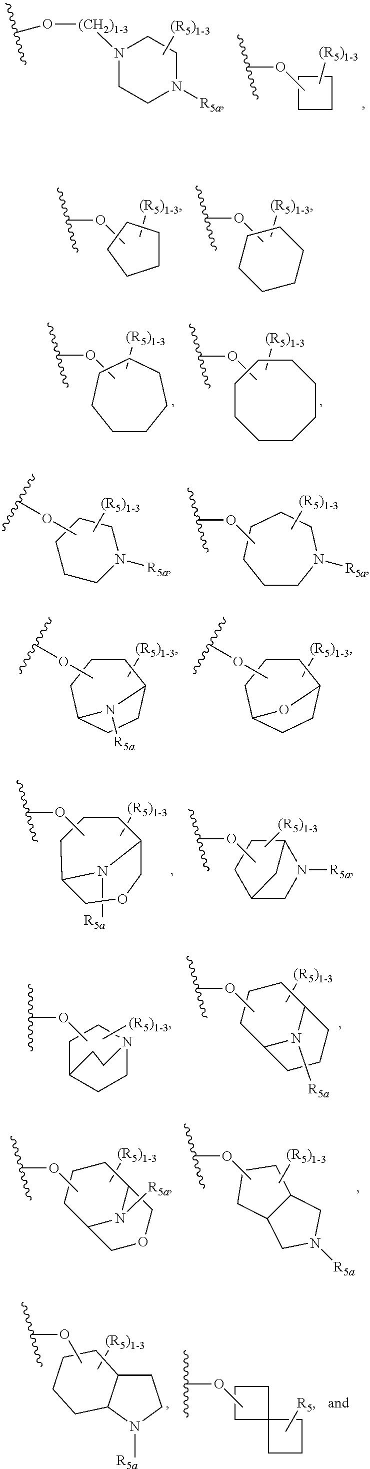

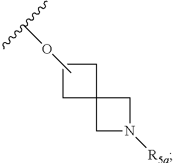

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R₆ is H; and R₆ₐ is —C(═O)CH₃, —C(═O)OCH₃, or —S(═O)₂CH₃.
In another embodiment of the compounds of Formula (I) or (II), R₁ is

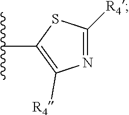

### R₂ is

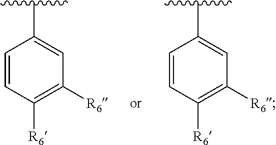

R₆ is H, F, Cl, CH₃; R₆″ is H, F, Cl; and R₆′ is CN, C(═O)NH₂,

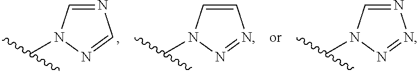 [SMILES: *n1cncn1 ; *n1ccnn1 ; *n1cnnn1]

R₄′ is H, CH₃, CHF₂, Et, iPr, or c-Pr; R₄″ is H, —O(CH₂)₁₋₃N(CH₃)₂,

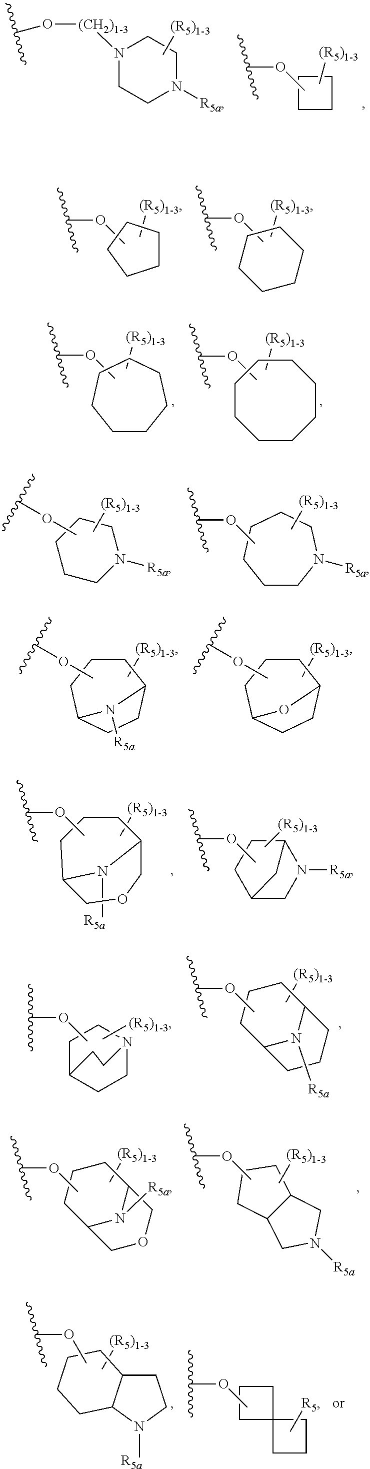

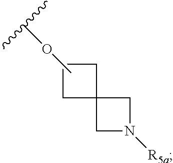

R₅ is H, CH₃, Et, i-Pr, F, OH, OMe, CH₂OH, CH₂CH₂OH, CMe₂OH, CH₂CMe₂OH, ═O; R₅ₐ is H, CH₃, Et, i-Pr, CH₂CH₂OH, CHMeCH₂OH, CH₂CMe₂OH, C(═O)CH₃, C(═O)OCH₃; R_{b} is H; and R₆ₐ is —C(═O)CH₃, —C(═O)OCH₃, or —S(═O)₂CH₃.
In one embodiment of the compounds of Formula (X), R₂ is

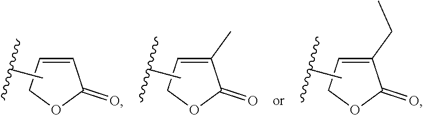

R₄′ is CH₃ or CD₃; one of R₁₀ is H, CH₃ or CD₃; the other R₁₀ is H, CH₃, CD₃,

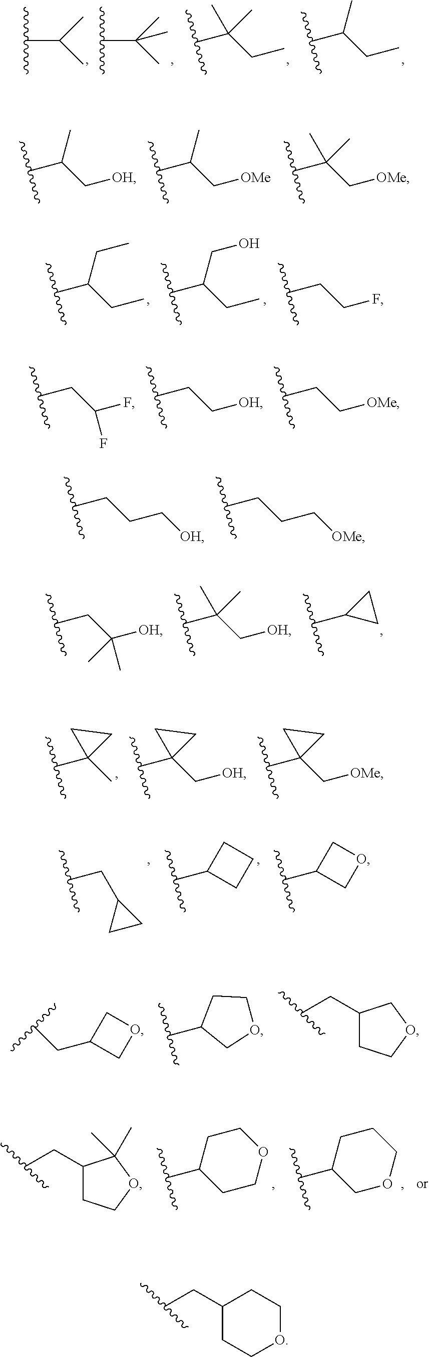

In another embodiment of the compounds of Formula (X), R₂ is

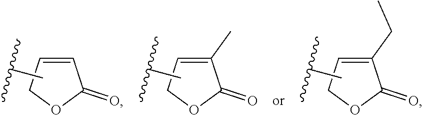

R₄′ is CH₃ or CD₃; R₁₀ and R₁₀ together with the nitrogen atom to which they are both attached form a heterocyclyl selected from the group consisting of

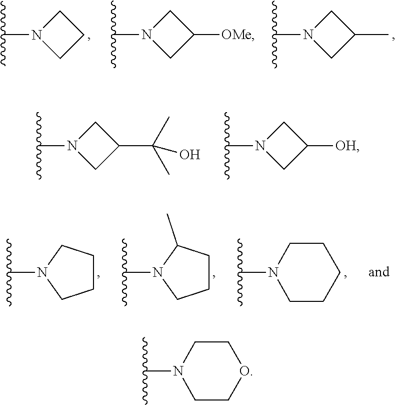

In another embodiment of the compounds of Formula (X), R₂ is

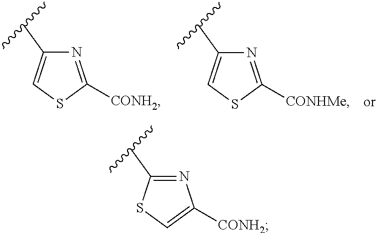

R₄′ is CH₃ or CD₃; one of R₁₀ is H, CH₃ or CD₃; the other R₁₀ is H, CH₃, CD₃; the other R₁₀ is H, CH₃, CD₃,

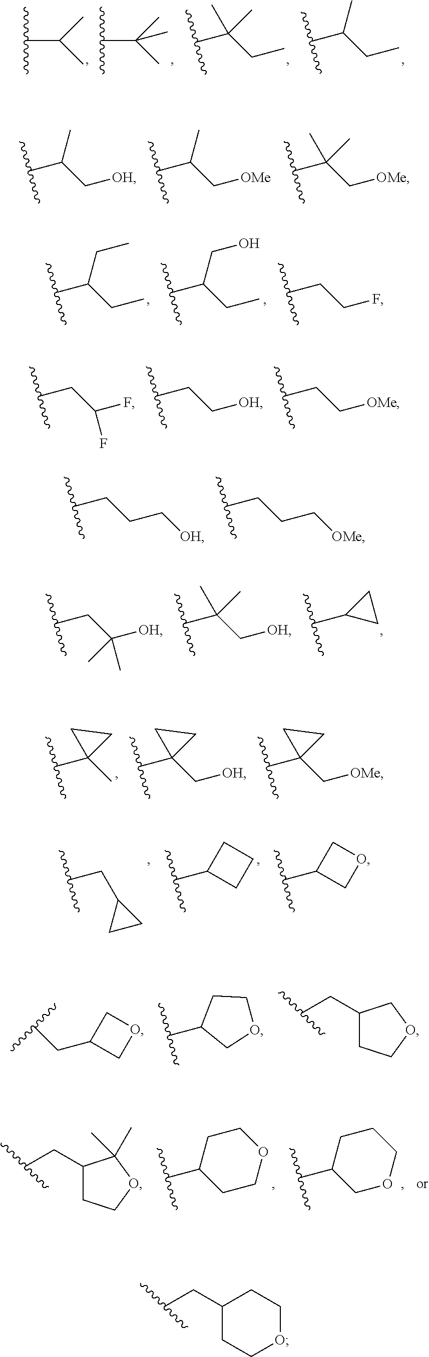

or R₁₀ and R₁₀ together with the nitrogen atom to which they are both attached form a heterocyclyl selected from the group consisting of

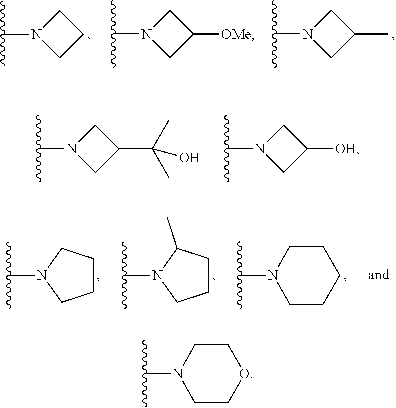

In another embodiment of the compounds of Formula (X), R₂ is

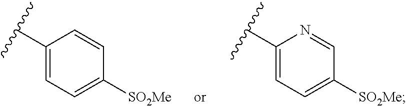 [SMILES: *c1ccc(S(C)(=O)=O)cc1 ; *c1ccc(S(C)(=O)=O)cn1]

R₄′ is CH₃ or CD₃; one of R₁₀ is H, CH₃ or CD₃; the other R₁₀ is H, CH₃, CD₃; the other R₁₀ is H, CH₃, CD₃,

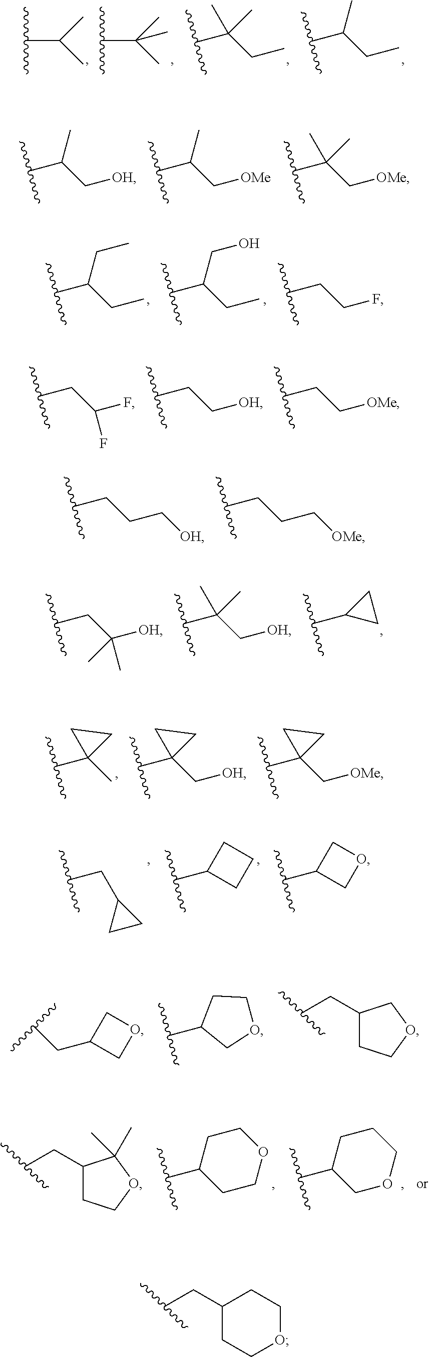

or R₁₀ and R₁₀ together with the nitrogen atom to which they are both attached form a heterocyclyl selected from the group consisting of

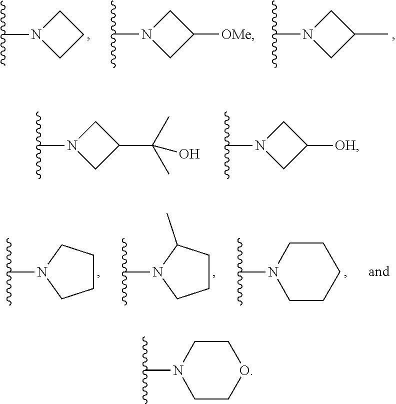

In another embodiment of the compounds of Formula (X), R₂ is

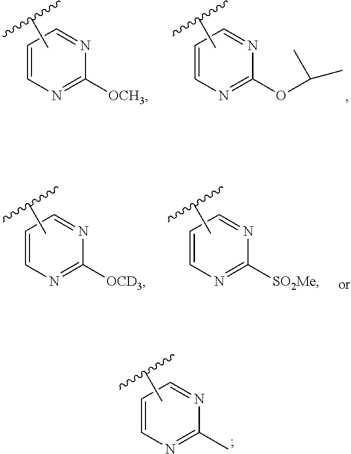

R₄′ is CH₃ or CH₃;
one of R₁₀ is H, CH₃ or CD₃; the other R₁₀ is H, CH₃, CD₃; the other R₁₀ is H, CH₃, CD₃,

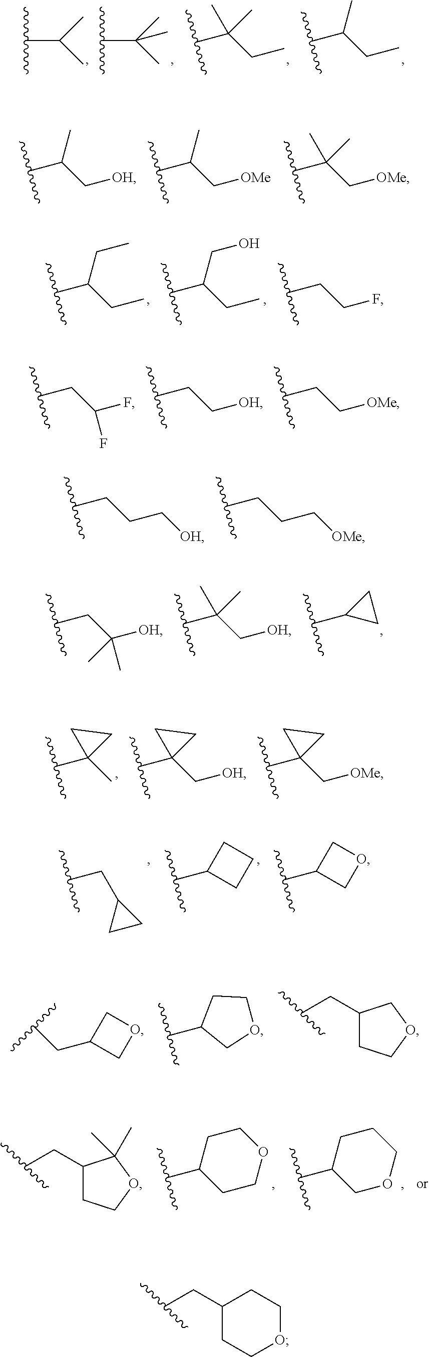

or R₁₀ and R₁₀ together with the nitrogen atom to which they are both attached form a heterocyclyl selected from the group consisting of

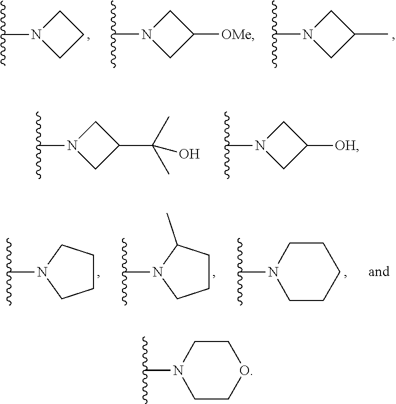

In another embodiment of the compounds of Formula (X), R₂ is

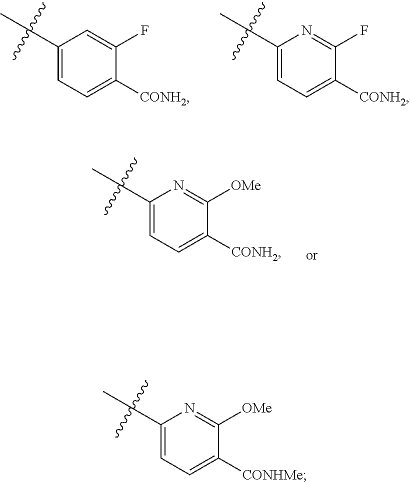

R₄′ is CH₃ or CD₃; one of R₁₀ is H, CH₃ or CD₃; the other R₁₀ is H, CH₃, CD₃; the other R₁₀ is H, CH₃, CD₃,

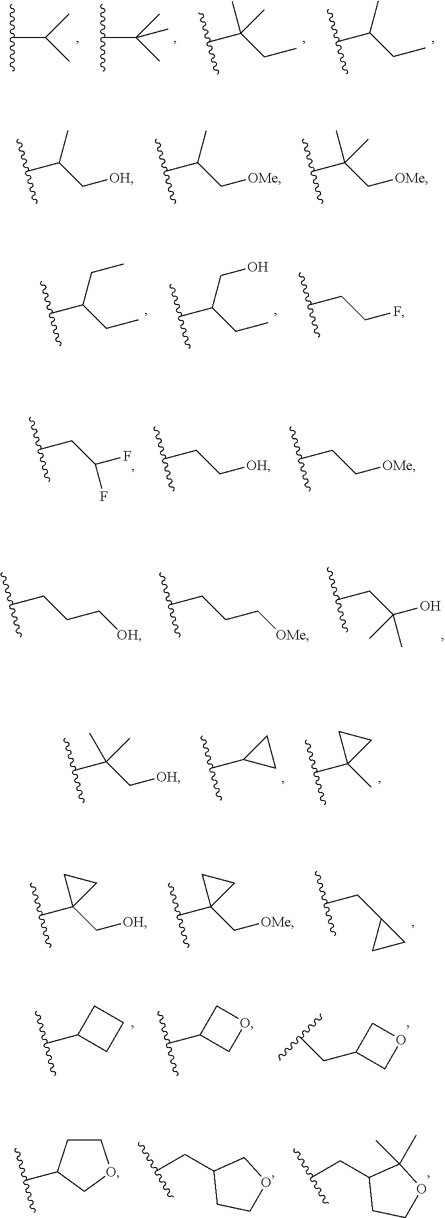

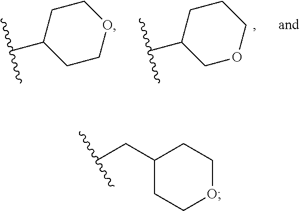 [SMILES: *C1CCOCC1 ; *C1CCCOC1 ; *CC1CCOCC1]

or R₁₀ and R₁₀ together with the nitrogen atom to which they are both attached form a heterocyclyl selected from the group consisting of

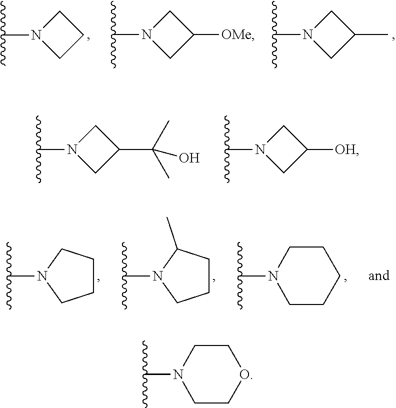

In another embodiment of the compounds of Formula (X), R₂ is

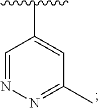 [SMILES: *c1cnnc(C)c1]

R₄′ is CH₃ or CD₃; one of R₁₀ is H, CH₃ or CD₃; the other R₁₀ is H, CH₃, CD₃; the other R₁₀ is H. CH₃, CD₃,

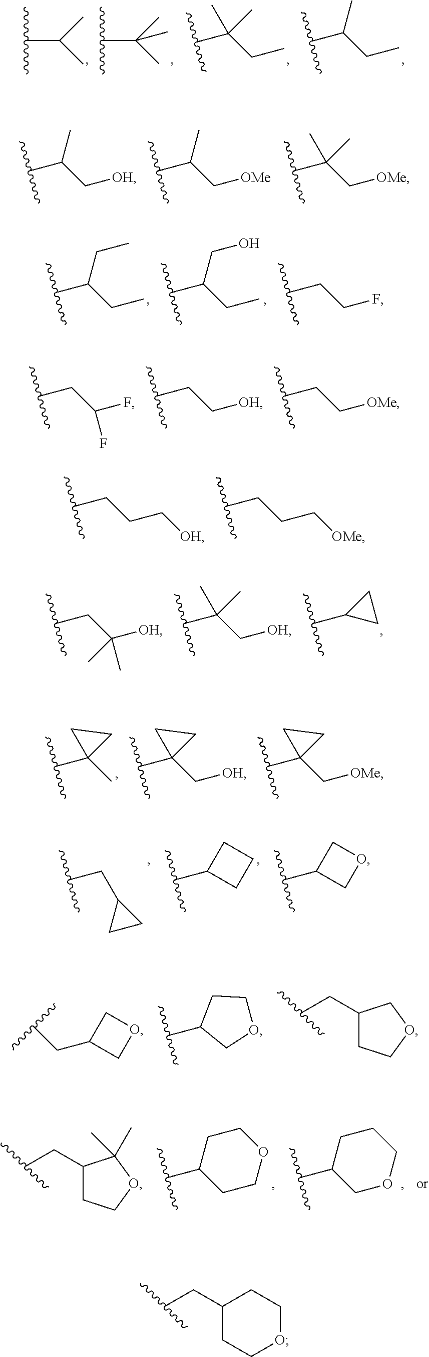

or R₁₀ and R₁₀ together with the nitrogen atom to which they are both attached form a heterocyclyl selected from the group consisting of

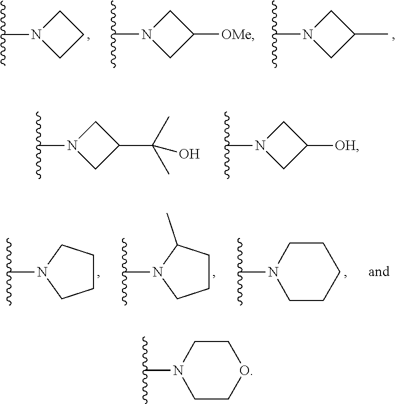

For a compound of Formulae (I)—(X), the scope of any instance of a variable substituent, including R₁, R₂, R₃, R₄ (R₄′ and R₄″), R₄, R₅, R₅ₐ, R_{5b}, R₆ (R₆′ and R₆″), R₆ₐ, R₇, R₇ₐ, R₈, R₉, R₁₀, Rₐ, R_{b}, R_{c}, R_{d}, Rₑ, R_{f}, and R_{g}, can be used independently with the scope of any other instance of a variable substituent. Variable substituents such as R₄′ and R₄″ or R₆′ and R₆″ represent a subset of the variable substituents R₄ and R₆, respectively. As such, the invention includes combinations of the different aspects.

In another embodiment, the compounds of the present invention have SGK1 IC₅₀ values≤10 μM.

In another embodiment, the compounds of the present invention have SGK1 IC₅₀ values≤1 μM.

In another embodiment, the compounds of the present invention have SGK1 IC₅₀ values≤0.5 μM.

In another embodiment, the compounds of the present invention have SGK1 IC₅₀ values≤0.1 μM.

In another embodiment, the compounds of the present invention have SGK1 IC₅₀ values≤0.05 μM.

In another embodiment, the compounds of the present invention have SGK1 IC₅₀ values≤0.01 μM.

### II. Other Embodiments of the Invention

In another embodiment, the present invention provides a pharmaceutical composition, comprising a pharmaceutically acceptable carrier and a therapeutically effective amount of at least one of the compounds of the present invention or a stereoisomer, a tautomer, a pharmaceutically acceptable salt, or a solvate thereof.

In another embodiment, the present invention provides a process for making a compound of the present invention.

In another embodiment, the present invention provides an intermediate for making a compound of the present invention.

In another embodiment, the present invention provides a pharmaceutical composition further comprising additional therapeutic agent(s).

In another embodiment, the present invention provides a method for the treatment and/or prophylaxis of a condition associated with aberrant SGK1 activity comprising administering to a patient in need of such treatment and/or prophylaxis a therapeutically effective amount of at least one of the compounds of the present invention or a stereoisomer, a tautomer, a pharmaceutically acceptable salt, or a solvate thereof. As used herein, the term “patient” encompasses all mammalian species.

The term “treating” or “treatment” as used herein refers to an approach for obtaining beneficial or desired results, including clinical results, by using a compound or a composition of the present invention. For purposes of this invention, beneficial or desired clinical results include, but are not limited to, one or more of the following: decreasing the severity and/or frequency one or more symptoms resulting from the disease, disorder, or condition; diminishing the extent of or causing regression of the disease, disorder, or condition; stabilizing the disease, disorder, or condition (e.g., preventing or delaying the worsening of the disease, disorder, or condition); delay or slowing the progression of the disease, disorder, or condition; ameliorating the disease, disorder, or condition state; decreasing the dose of one or more other medications required to treat the disease, disorder, or condition; and/or increasing the quality of life.

As used herein, “prophylaxis” is the protective treatment of a disease state to reduce and/or minimize the risk and/or reduction in the risk of recurrence of a disease state by administering to a patient a therapeutically effective amount of at least one of the compounds of the present invention or a or a stereoisomer, a tautomer, a pharmaceutically acceptable salt, or a solvate thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, “prevention” covers the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment.

### III. Chemistry

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt, a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention. As used herein, “a compound of the invention” or “compounds of the invention” means one or more compounds encompassed by Formulae (I)—(VII), and any subgenus and exemplified species thereof.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

In accordance with a convention used in the art, a bond pointing to a wave line, such as

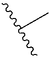

used in structural formulas herein, depicts the bond that is the point of attachment of the moiety or substituent to the core or backbone structure.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Alkyl group can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). “Alkyl” also includes deuteroalkyl such as CD₃.

“Heteroalkyl” refers to an alkyl group where one or more carbon atoms have been replaced with a heteroatom, such as, O, N, or S. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) and the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

“Alkenyl” or “alkenylene” is intended to include hydrocarbon chains of either straight or branched configuration having the specified number of carbon atoms and one or more, preferably one to two, carbon-carbon double bonds that may occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3, pentenyl, 4-pentenyl, 2-hexenyl, 3-hexenyl, 4-hexenyl, 5-hexenyl, 2-methyl-2-propenyl, and 4-methyl-3-pentenyl.

“Alkynyl” or “alkynylene” is intended to include hydrocarbon chains of either straight or branched configuration having one or more, preferably one to three, carbon-carbon triple bonds that may occur in any stable point along the chain. For example. “C₂₋₆ alkynyl” (or alkynylene), is intended to include C₂, C₃, C₄, C₅, and C₆ alkynyl groups; such as ethynyl, propynyl, butynyl, pentynyl, and hexynyl.

As used herein, “arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group can comprise 7 to 20 carbon atoms, e.g., the alkyl moiety is 1 to 6 carbon atoms and the aryl moiety is 6 to 14 carbon atoms.

The term “benzyl”, as used herein, refers to a methyl group on which one of the hydrogen atoms is replaced by a phenyl group, wherein said phenyl group may optionally be substituted with 1 to 5 groups, preferably 1 to 3 groups, OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and CO₂CH₃. “Benzyl” can also be represented by formula “Bn”.

The term “lower alkoxy”, “alkoxy” or “alkyloxy”, “aryloxy” or “aralkoxy” refers to any of the above alkyl, aralkyl or aryl groups linked to an oxygen atom. “C₁ to C₆ alkoxy” or “C₁₋₆ alkoxy” (or alkyloxy), is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “lower alkylthio”, “alkylthio”, “thioalkoxy”, “arylthio”, or “aralkylthio” represents an alkyl, aryl, or aralkyl group as defined above with the indicated number of carbon atoms attached through a sulphur bridge; for example methyl-S— and ethyl-S—.

The term “halogen” or “halo” as used herein alone or as part of another group refers to chlorine, bromine, fluorine, and iodine, with chlorine or fluorine being preferred.

“Haloalkyl” is intended to include both branched and straight-chain saturated aliphatic hydrocarbon groups having the specified number of carbon atoms, substituted with one or more halogens. “C₁ to C₆ haloalkyl” or “C₁₋₆ haloalkyl” (or haloalkyl), is intended to include C₁, C₂, C₃, C₄, C₅, and C₆ haloalkyl groups. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with 1 or more fluorine atoms. The term “polyhaloalkyl” as used herein refers to an “alkyl” group as defined above which includes from 2 to 9, preferably from 2 to 5, halo substituents, such as F or Cl, preferably F, such as polyfluoroalkyl, for example, CF₃CH₂, CF₃ or CF₃CF₂CH₂.

“Haloalkoxy” or “haloalkyloxy” represents a haloalkyl group as defined above with the indicated number of carbon atoms attached through an oxygen bridge. For example, “C₁₋₆ haloalkoxy”, is intended to include C₁, C₂, C₃, C₄, C₅, and C₆ haloalkoxy groups. Examples of haloalkoxy include, but are not limited to, trifluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—. The term “polyhaloalkyloxy” as used herein refers to an “alkoxy” or “alkyloxy” group as defined above which includes from 2 to 9, preferably from 2 to 5, halo substituents, such as F or Cl, preferably F, such as polyfluoroalkoxy, for example, CF₃CH₂O, CF₃O or CF₃CF₂CH₂O.

“Hydroxyalkyl” is intended to include both branched and straight-chain saturated aliphatic hydrocarbon groups having the specified number of carbon atoms, substituted with 1 or more hydroxyl (OH). For example, “C₁₋₆ hydroxyalkyl” (or hydroxyalkyl), is intended to include C₁, C₂, C₃, C₄, C₅, and C₆ hydroxyalkyl groups.

The term “cycloalkyl” refers to cyclized alkyl groups, including mono-, bi- or poly-cyclic ring systems. “C₃ to C₇ cycloalkyl” or “C₃₋₇ cycloalkyl” is intended to include C₃, C₄, C₅, C₆, and C₇ cycloalkyl groups. Example cycloalkyl groups include, but are not limited to, cyclopropyl, cyclobutyl, cyclopentyl, cyclohexyl, and norbornyl. Branched cycloalkyl groups such as 1-methylcyclopropyl and 2-methylcyclopropyl are included in the definition of “cycloalkyl”.

As used herein, “carbocycle”. “carbocyclyl”, or “carbocyclic” is intended to mean any stable 3-, 4-, 5-, 6-, 7-, or 8-membered monocyclic or 5-, 6-, 7-, 8-, 9-, 10-, 11-, 12-, or 13-membered polycyclic (including bicyclic or tricyclic) hydrocarbon ring, any of which may be saturated or partially unsaturated. That is, the term “carbocycle”, “carbocyclyl”, or “carbocyclic” includes, without limitation, cycloalkyl and cycloalkenyl. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane (decalin), [2.2.2]bicyclooctane, fluorenyl, indanyl, adamantyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, indanyl, and tetrahydronaphthyl. A bridged ring occurs when one or more, preferably one to three, carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge.

Furthermore, the term “carbocyclyl”, including “cycloalkyl” and “cycloalkenyl”, as employed herein alone or as part of another group includes saturated or partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl and cyclododecyl, cyclohexenyl, any of which groups may be optionally substituted with 1 to 4 substituents such as halogen, alkyl, alkoxy, hydroxy, aryl, aryloxy, arylalkyl, cycloalkyl, alkylamido, alkanoylamino, oxo, acyl, arylcarbonylamino, nitro, cyano, thiol and/or alkylthio and/or any of the alkyl substituents.

As used herein, the term “bicyclic carbocycle” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated or partially unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, 1,2-dihydronaphthyl, 1,2,3,4-tetrahydronaphthyl, and indanyl.

As used herein, the term “aryl”, as employed herein alone or as part of another group, refers to monocyclic or polycyclic (including bicyclic and tricyclic) aromatic hydrocarbons, including, for example, phenyl, naphthyl, anthracenyl, and phenanthranyl. Aryl moieties are well known and described, for example, in Lewis, R. J., ed., Hawley's Condensed Chemical Dictionary, 13th Edition, John Wiley & Sons, Inc., New York (1997). In one embodiment, the term “aryl” denotes monocyclic and bicyclic aromatic groups containing 6 to 10 carbons in the ring portion (such as phenyl or naphthyl including 1-naphthyl and 2-naphthyl). For example, “C₆ or C₁₀ aryl” refers to phenyl and naphthyl.

As used herein, the term “heterocycle”. “heterocyclyl”, or “heterocyclic group” is intended to mean a stable 3-, 4-, 5-, 6-, or 7-membered monocyclic or 5-, 6-, 7-, 8-, 9-, 10-, 11-, 12-, 13-, or 18-membered polycyclic (including bicyclic and tricyclic) heterocyclic ring that is saturated, or partially unsaturated, and that contains carbon atoms and 1, 2, 3 or 4 heteroatoms independently selected from N, O and S; and including any polycyclic group in which any of the above-defined heterocyclic rings is fused to a carbocyclic or an aryl (e.g., benzene) ring. That is, the term “heterocycle”, “heterocyclyl”, or “heterocyclic group” includes non-aromatic ring systems, such as heterocycloalkyl and heterocycloalkenyl. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. Examples of hetercyclyl include, without limitation, azetidinyl, piperazinyl, piperidinyl, piperidonyl, piperonyl, pyranyl, morpholinyl, tetrahydrofuranyl, tetrahydroisoquinolinyl, tetrahydroquinolinyl, morpholinyl, dihydrofuro[2,3-b]tetrahydrofuran.

As used herein, the term “bicyclic heterocycle” or “bicyclic heterocyclic group” is intended to mean a stable 9- or 10-membered heterocyclic ring system which contains two fused rings and consists of carbon atoms and 1, 2, 3, or 4 heteroatoms independently selected from N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocycle, a 6-membered heterocycle or a carbocycle (provided the first ring is not benzo when the second ring is a carbocycle).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocycle exceeds 1, then these heteroatoms are not adjacent to one another. It is preferred that the total number of S and O atoms in the heterocycle is not more than 1. Examples of a bicyclic heterocyclic group are, but not limited to, 1,2,3,4-tetrahydroquinolinyl, 1,2,3,4-tetrahydroisoquinolinyl, 5,6,7,8-tetrahydro-quinolinyl, 2,3-dihydro-benzofuranyl, chromanyl, 1,2,3,4-tetrahydro-quinoxalinyl, and 1,2,3,4-tetrahydro-quinazolinyl.

Bridged rings are also included in the definition of heterocycle. A bridged ring occurs when one or more, preferably one to three, atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge.

As used herein, the term “heteroaryl” is intended to mean stable monocyclic and polycyclic (including bicyclic and tricyclic) aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

Examples of heteroaryl include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathianyl, phenoxazinyl, phthalazinyl, pteridinyl, purinyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Examples of 5- to 10-membered heteroaryl include, but are not limited to, pyridinyl, furanyl, thienyl, pyrazolyl, imidazolyl, imidazolidinyl, indolyl, tetrazolyl, isoxazolyl, oxazolyl, oxadiazolyl, oxazolidinyl, thiadiazinyl, thiadiazolyl, thiazolyl, triazinyl, triazolyl, benzimidazolyl, 1H-indazolyl, benzofuranyl, benzothiofuranyl, benztetrazolyl, benzotriazolyl, benzisoxazolyl, benzoxazolyl, oxindolyl, benzoxazolinyl, benzthiazolyl, benzisothiazolyl, isatinoyl, isoquinolinyl, octahydroisoquinolinyl, isoxazolopyridinyl, quinazolinyl, quinolinyl, isothiazolopyridinyl, thiazolopyridinyl, oxazolopyridinyl, imidazolopyridinyl, and pyrazolopyridinyl. Examples of 5- to 6-membered heterocycles include, but are not limited to, pyridinyl, furanyl, thienyl, pyrrolyl, pyrazolyl, pyrazinyl, imidazolyl, imidazolidinyl, indolyl, tetrazolyl, isoxazolyl, oxazolyl, oxadiazolyl, oxazolidinyl, thiadiazinyl, thiadiazolyl, thiazolyl, triazinyl, and triazolyl.

It is understood herein that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

When a dotted ring is used within a ring structure, this indicates that the ring structure may be saturated, partially saturated or unsaturated.

When a bond to a substituent is shown to cross a bond connecting two atoms in a ring, then such substituent may be bonded to any atom on the ring. For example, when the ring has a bicyclic or tricyclic structure, then such substituent may be bonded to any ring-member atom of the bicyclic or tricyclic structure. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

One skilled in the art will recognize that substituents and other moieties of the compounds of the present invention should be selected in order to provide a compound which is sufficiently stable to provide a pharmaceutically useful compound which can be formulated into an acceptably stable pharmaceutical composition. Compounds of the present invention which have such stability are contemplated as falling within the scope of the present invention.

The term “counter ion” is used to represent a negatively charged species such as chloride, bromide, hydroxide, acetate, and sulfate. The term “metal ion” refers to alkali metal ions such as sodium, potassium or lithium and alkaline earth metal ions such as magnesium and calcium, as well as zinc and aluminum.

As referred to herein, the term “substituted” means that at least one hydrogen atom is replaced with a non-hydrogen group, provided that normal valencies are maintained and that the substitution results in a stable compound. When a substituent is keto, also known as “oxo” which is a substituent oxygen atom connected to another atom by a double bond (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0, 1, 2, or 3 R groups, then said group be unsubstituted when it is substituted with 0 R group, or be substituted with up to three R groups, and at each occurrence R is selected independently from the definition of R.

Also, combinations of substituents and/or variables are permissible only if such combinations result in stable compounds.

As used herein, the term “tautomer” refers to each of two or more isomers of a compound that exist together in equilibrium, and are readily interchanged by migration of an atom or group within the molecule For example, one skilled in the art would readily understand that a 1,2,3-triazole exists in two tautomeric forms as defined above:

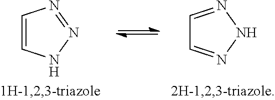 [SMILES: c1c[nH]nn1 ; c1cn[nH]n1]

Thus, this disclosure is intended to cover all possible tautomers even when a structure depicts only one of them.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

The compounds of the present invention can be present as salts, which are also within the scope of this invention. Pharmaceutically acceptable salts are preferred. As used herein, “pharmaceutically acceptable salts” refer to derivatives of the disclosed compounds wherein the parent compound is modified by making acid or base salts thereof. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two, generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA (1990), the disclosure of which is hereby incorporated by reference.

If the compounds of the present invention have, for example, at least one basic center, they can form acid addition salts. These are formed, for example, with strong inorganic acids, such as mineral acids, for example sulfuric acid, phosphoric acid or a hydrohalic acid, with organic carboxylic acids, such as alkanecarboxylic acids of 1 to 4 carbon atoms, for example acetic acid, which are unsubstituted or substituted, for example, by halogen as chloroacetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or p-toluene-sulfonic acid. Corresponding acid addition salts can also be formed having, if desired, an additionally present basic center. The compounds of the present invention having at least one acid group (for example COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono, di or tri-lower alkylamine, for example ethyl, tert-butyl, diethyl, diisopropyl, triethyl, tributyl or dimethyl-propylamine, or a mono, di or trihydroxy lower alkylamine, for example mono, di or triethanolamine. Corresponding internal salts may furthermore be formed. Salts which are unsuitable for pharmaceutical uses but which can be employed, for example, for the isolation or purification of free compounds of Formula (I) or their pharmaceutically acceptable salts, are also included.

Preferred salts of the compounds of Formula (I) which contain a basic group include monohydrochloride, hydrogensulfate, methanesulfonate, phosphate, nitrate or acetate.

Preferred salts of the compounds of Formula (I) which contain an acid group include sodium, potassium and magnesium salts and pharmaceutically acceptable organic amines.

In addition, the compounds of the present invention may have prodrug forms. Any compound that will be converted in vivo to provide the bioactive agent, i.e., a compound of formula (I), is a prodrug within the scope and spirit of the invention. Preparation of prodrugs is well known in the art and described in, for example, King, F. D., ed., Medicinal Chemistry: Principles and Practice, The Royal Society of Chemistry, Cambridge, UK (1994); Testa, B. et al., Hydrolysis in Drug and Prodrug Metabolism, Chemistry, Biochemistry and Enzymology, VCHA and Wiley-VCH, Zurich, Switzerland (2003); Wermuth, C. G., ed., The Practice of Medicinal Chemistry, Academic Press, San Diego, CA (1999).

The present invention is intended to include all isotopes of atoms occurring in the present compounds. Isotopes include those atoms having the same atomic number but different mass numbers. By way of general example and without limitation, isotopes of hydrogen include deuterium (²H or D) and tritium (³H or T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds have a variety of potential uses, e.g., as standards and reagents in determining the ability of a potential pharmaceutical compound to bind to target proteins or receptors, or for imaging compounds of this invention bound to biological receptors in vivo or in vitro.

“Stable compound” and “stable structure” are meant to indicate a compound that is sufficiently robust to survive isolation to a useful degree of purity from a reaction mixture, and formulation into an efficacious therapeutic agent. It is preferred that compounds of the present invention do not contain a N-halo, S(O)₂H, or S(O)H group.

The term “solvate” means a physical association of a compound of this invention with one or more solvent molecules, whether organic or inorganic. This physical association includes hydrogen bonding. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

Abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “saturated” for saturated, “MW” for molecular weight, “mp” for melting point, “ee” for enantiomeric excess, “MS” or “Mass Spec” for mass spectrometry. “ESI” for electrospray ionization mass spectroscopy, “HR” for high resolution, “HRMS” for high resolution mass spectrometry, “LCMS” for liquid chromatography mass spectrometry. “HPLC” for high pressure liquid chromatography. “RP HPLC” for reverse phase HPLC, “TLC” or “tlc” for thin layer chromatography, “NMR” for nuclear magnetic resonance spectroscopy, “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.
Me Methyl
Et Ethyl
Pr Propyl
i-Pr Isopropyl
Bu Butyl
i-Bu Isobutyl
t-Bu tert-butyl
Ph Phenyl
Bn Benzyl
Boc tert-butyloxycarbonyl
ACN acetonitrile
AcOH or HOAc acetic acid
AlCl₃ aluminum chloride
AIBN Azobisisobutyronitrile
BBr₃ boron tribromide
BCl₃ boron trichloride
BOP reagent benzotriazol-1-yloxytris(dimethylamino)phosphonium hexafluorophosphate
Burgess reagent [methoxycarbonylsulfamoyl]triethylammonium hydroxide
CBz Carbobenzyloxy
CH₂Cl₂ Dichloromethane
CH₃CN or ACN Acetonitrile
CDCl₃ deutero-chloroform
CHCl₃ Chloroform
mCPBA or m-CPBA meta-chloroperbenzoic acid
Cs₂CO₃ cesium carbonate
Cu(OAc)₂ copper (II) acetate
DABCO 1,4-diazabicyclo[2.2.2]octane
DBU 1,8-diazabicyclo[5.4.0]undec-7-ene
DCE 1,2 dichloroethane
DCM dichloromethane
DEA diethylamine
Dess-Martin 1,1,1-tris(acetyloxy)-1,1-dihydro-1,2-beniziodoxol-3-(1H)-one
DIEA, or Hunig's base diisopropylethylamine
DMA or DMAc dimethylacetamide
DMAP 4-dimethylaminopyridine
DME 1,2-dimethoxyethane
DMF dimethyl formamide
DMSO dimethyl sulfoxide
dppf 1,1′-Ferrocenediyl-bis(diphenylphosphine)
EDC N-(3-dimethylaminopropyl)-N′-ethylcarbodiimide
EDCI N-(3-dimethylaminopropyl)-N′-ethylcarbodiimide hydrochloride
EDTA ethylenediaminetetraacetic acid
Et₃N or TEA triethylamine
EtOAc ethyl acetate
Et₂O diethyl ether
EOH Ethanol
HCl hydrochloric acid
HATU O-(7-azabenzotriazol-1-yl)-N,N,N′,N′-tetramethyluronium
hexafluorophosphate
Hex Hexane
HOBt or HOBT 1-hydroxybenzotriazole
H₂SO₄ sulfuric acid
Ir[dF(CF₃)ppy]₂(dtbbpy) PF₆ [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
K₂CO₃ potassium carbonate
KOAc potassium acetate
K₃PO₄ potassium phosphate
LAH lithium aluminum hydride
LED light-emitting diode
LG leaving group
LiOH lithium hydroxide
MeCN acetonitrile
MeOH methanol
MgSO₄ magnesium sulfate
MsCl methylsulfonyl chloride
MsOH or MSA methylsulfonic acid
NaCl sodium chloride
NaH sodium hydride
NaHCO₃ sodium bicarbonate
Na₂CO₃ sodium carbonate
NaOH sodium hydroxide
Na₂SO₃ sodium sulfite
Na₂SO₄ sodium sulfate
NBS N-bromosuccinimide
NCS N-chlorosuccinimide
NH₃ Ammonia
NH₄Cl ammonium chloride
NH₄OH ammonium hydroxide
NIS N-iodosuccinimide
OTf triflate or trifluoromethanesulfonate
Pd₂(dba)₃ tris(dibenzylideneacetone)dipalladium(0)
Pd(OAc)₂ palladium(II) acetate
Pd(PPh₃)₄ tetrakis(triphenylphosphine)palladium(0)
Pd/C palladium on carbon
PdCl₂(dppf) [1,1′-bis(diphenylphosphino)-ferrocene]dichloropalladium(II)
Pet ether petroleum ether
PG protecting group
POCl₃ phosphorus oxychloride
i-PrOH or IPA isopropanol
PyBOP benzotriazol-1-yloxytripyrrolidinophosphonium
hexafluorophosphate
SEM-Cl 2-(trimethysilyl)ethoxymethyl chloride
SFC supercritical fluid chromatography
SiO₂ silica oxide
TEA triethylamine
TFA trifluoroacetic acid
THF tetrahydrofuran
TMSCHN₂ trimethylsilyldiazomethane
T3P® propane phosphonic acid anhydride

The compounds of the present invention can be prepared in a number of ways known to one skilled in the art of organic synthesis.

### IV. Biology

The inhibitory activity of compounds in SGK1 was determined by one of two assays.

Assay A was carried out in 20 mM Hepes pH 7.5, 10 mM MgCl₂, 0.05 mg/ml BSA, 0.015% Brij-15 and 2 mM DTT. Incubation mixtures containing 125 μM full length SGK1 (Life Technologies Part #PR7358A), 20 μM ATP at Km, and 1.5 mM peptide substrate ([FITC]-AHA-KKRNRRSVA-[OH]) were incubated for 40 min., after which they were quenched with 1 mM EDTA solution. The reaction mixture was analyzed on a Caliper LabChip 3000 (Caliper LifeSciences, Hopinkton, MA, USA) by electrophoretic separation of the fluorescent substrate and phosphorylated product using the following run conditions: pressure of −0.7 psi, downstream voltage of −2200 V, and upstream voltage of −500 V.

In Assay B, 0.67 μL compound+20 μL of 40 μM ATP+20 μM Peptide: [FITC]-AHA-KKRNRRSVA-[OH]) were added in 20 mM Hepes+10 mM MgCl₂ 1+0015% Brij-35+4 mM DTT+0.05 mg/ml BSA. 20 μl of 2 nM SGK1 were added in 20 mM Hepes+10 mM MgCl₂+0015% Brij-35+4 mM DTT+0.05 mg/ml BSA and the reaction incubated for 60 min at room temperature. The enzymatic reaction was quenched by addition 10 μl of 1% Formic Acid+150 nM Internal Standard: FITC-(Ahx)KKRNRR(pS)VA\hat{ }—OH (final concentration). The plates are spun down at 3700 RPM for 10 minutes to remove all precipitate. Samples were loaded on to the Sciex Echo Mass Spec attached to a Sciex 6500 equipped with ESI and was used to analyze the phosphorylated peptide product. MRM transition of product and internal standard were monitored at m/z 755 to 937.5 and m/z 757.7 to 941.3 respectively. Peak area of analyte was normalized with internal standard.

Inhibition data were calculated from the product conversion generated by the no enzyme control reactions for 100% inhibition and vehicle-only reactions for 0% inhibition. Dose response curves were generated to determine the concentration required for inhibiting 50% of the enzyme activity. Compounds were dissolved at 10 mM in dimethylsulfoxide (DMSO) and evaluated at eleven concentrations.

Representative Examples were tested in the SGK1 assays (Assay A or B) described above and found having SGK1 inhibitory activity. Their SGK1 inhibitory activity (IC₅₀ values) of 3.6 μM (3,600 nM) was observed and shown in Table A below along with the assay used.

TABLE A

| Example No | SGK1 IC₅₀ |
|---|---|
| 1 | A, 2990 |
| 2 | A, 81 |
| 3 | A, 239 |
| 4 | A, 33 |
| 5 | A, 73 |
| 6 | A, 157 |
| 7 | A, 90 |
| 8 | A, 67 |
| 9 | A, 263 |
| 11 | A, 1751 |
| 13 | A, 28 |
| 14 | A, 941 |
| 15 | A, 499 |
| 16 | A, 324 |
| 17 | A, 1299 |
| 19 | A, 274 |
| 20 | A, 47 |
| 21 | A, 13 |
| 22 | A, 12 |
| 23 | A, 11 |
| 24 | A, 41 |
| 25 | A, 22 |
| 26 | A, 19 |
| 27 | A, 12 |
| 28 | A, 32 |
| 29 | A, 181 |
| 30 | A, 65 |
| 31 | A, 112 |
| 32 | A, 11 |
| 33 | A, 9 |
| 34 | A, 24 |
| 35 | A, 21 |
| 36 | A, 41 |
| 37 | A, 136 |
| 38 | A, 22 |
| 39 | A, 48 |
| 40 | A, 115 |
| 41 | A, 5 |
| 42 | A, 187 |
| 43 | A, 79 |
| 44 | A, 735 |
| 45 | A, 28 |
| 46 | A, 10 |
| 47 | A, 44 |
| 48 | A, 307 |
| 49 | A, 43 |
| 50 | A, 69 |
| 51 | A, 13 |
| 52 | A, 210 |
| 53 | A, 15 |
| 54 | A, 596 |
| 55 | A, 344 |
| 55 | A, 8 |
| 56 | A, 486 |
| 57 | A, 112 |
| 58 | A, 854 |
| 59 | A, 10 |
| 60 | A, 130 |
| 61 | A, 33 |
| 62 | A, 111 |
| 63 | A, 105 |
| 64 | A, 285 |
| 65 | A, 19 |
| 66 | A, 39 |
| 67 | A, 261 |
| 68 | A, 287 |
| 69 | A, 37 |
| 70 | A, 21 |
| 71 | A, 6 |
| 72 | A, 13 |
| 73 | A, 8 |
| 74 | A, 25 |
| 75 | A, 44 |
| 76 | A, 53 |
| 77 | A, 31 |
| 78 | A, 67 |
| 79 | A, 26 |
| 80 | A, 14 |
| 81 | A, 32 |
| 82 | A, 15 |
| 83 | A, 48 |
| 84 | A, 19 |
| 85 | A, 27 |
| 86 | A, 9 |
| 87 | A, 2 |
| 88 | A, 8 |
| 89 | A, 6 |
| 90 | A, 49 |
| 91 | A, 10 |
| 92 | A, 10 |
| 93 | A, 5 |
| 94 | A, 3 |
| 95 | A, 6 |
| 96 | A, 8 |
| 97 | A, 3 |
| 98 | A, 14 |
| 99 | A, 7 |
| 100 | A, 8 |
| 101 | A, 21 |
| 102 | A, 45 |
| 103 | A, 726 |
| 104 | A, 247 |
| 105 | A, 335 |
| 106 | A, 823 |
| 112 | A, 4 |
| 113 | A, 23 |
| 114 | A, 16 |
| 115 | A, 103 |
| 116 | A, 12 |
| 117 | A, 6 |
| 118 | A, 130 |
| 119 | A, 18 |
| 120 | A, 3405 |
| 121 | A, 383 |
| 122 | A, 29 |
| 123 | A, 606 |
| 124 | A, 2 |
| 125 | A, 27 |
| 126 | A, 5 |
| 127 | A, 5 |
| 128 | A, 2 |
| 129 | A, 2 |
| 130 | A, 19 |
| 131 | A, 2 |
| 132 | A, 4 |
| 133 | A, 4 |
| 134 | A, 2 |
| 135 | A, 3 |
| 136 | A, 230 |
| 137 | A, 143 |
| 138 | A, 7 |
| 139 | A, 3 |
| 140 | A, 6 |
| 141 | A, 3 |
| 142 | A, 9 |
| 143 | A, 10 |
| 144 | A, 24 |
| 145 | A, 765 |
| 146 | A, 835 |
| 147 | A, 4 |
| 148 | A, 5 |
| 149 | A, 1 |
| 150 | A, 5 |
| 151 | A, 285 |
| 152 | A, 5 |
| 153 | A, 1 |
| 154 | A, 7 |
| 155 | A, 2 |
| 156 | A, 3 |
| 157 | A, 15 |
| 158 | A, 6 |
| 159 | A, 17 |
| 160 | A, 20 |
| 161 | A, 2 |
| 162 | A, 5 |
| 163 | A, 6 |
| 164 | A, 2 |
| 165 | A, 284 |
| 166 | A, 3 |
| 167 | A, 1 |
| 169 | A, 12 |
| 170 | A, 6 |
| 171 | A, 16 |
| 172 | A, 350 |
| 173 | A, 4 |
| 174 | A, 37 |
| 175 | A, 4 |
| 176 | A, 4 |
| 177 | A, 5 |
| 178 | A, 6 |
| 179 | A, 8 |
| 180 | A, 47 |
| 181 | A, 17 |
| 182 | A, 35 |
| 183 | A, 42 |
| 184 | A, 36 |
| 185 | A, 43 |
| 186 | A, 3 |
| 187 | A, 6 |
| 188 | A, 4 |
| 189 | A, 2 |
| 190 | A, 19 |
| 191 | A, 6 |
| 192 | A, 10 |
| 193 | A, 25 |
| 194 | A, 188 |
| 195 | A, 107 |
| 196 | A, 222 |
| 197 | A, 30 |
| 198 | A, 2 |
| 199 | A, 2 |
| 200 | A, 15 |
| 201 | A, 3 |
| 202 | A, 12 |
| 203 | A, 19 |
| 204 | A, 5 |
| 205 | A, 7 |
| 206 | A, 12 |
| 207 | A, 4 |
| 208 | A, 20 |
| 209 | A, 3 |
| 210 | A, 9 |
| 211 | A, 3 |
| 212 | A, 6 |
| 213 | A, 5 |
| 214 | A, 11 |
| 215 | A, 6 |
| 216 | A, 26 |
| 217 | A, 62 |
| 218 | A, 233 |
| 219 | A, 68 |
| 220 | A, 7 |
| 221 | A, 800 |
| 222 | A, 43 |
| 223 | A, 199 |
| 224 | A, 11 |
| 225 | A, 15 |
| 226 | A, 2 |
| 227 | A, 18 |
| 228 | A, 6 |
| 229 | A, 46 |
| 230 | A, 21 |
| 231 | A, 12 |
| 232 | A, 161 |
| 233 | A, 33 |
| 234 | A, 64 |
| 235 | A, 37 |
| 236 | A, 19 |
| 237 | A, 4 |
| 238 | A, 91 |
| 239 | A, 43 |
| 240 | A, 14 |
| 241 | A, 0.3 |
| 242 | A, 5 |
| 243 | A, 1 |
| 244 | A, 1 |
| 245 | A, 1 |
| 246 | A, 13 |
| 247 | A, 12 |
| 248 | A, 3 |
| 249 | A, 1 |
| 250 | A, 3 |
| 251 | A, 1 |
| 252 | A, 4 |
| 253 | A, 2 |
| 254 | A, 23 |
| 255 | A, 10 |
| 256 | A, 0.5 |
| 257 | A, 65 |
| 258 | A, 67 |
| 259 | A, 28 |
| 260 | A, 48 |
| 261 | A, 32 |
| 262 | A, 18 |
| 263 | A, 12 |
| 264 | A, 12 |
| 265 | A, 18 |
| 266 | A, 18 |
| 267 | A, 9 |
| 268 | A, 38 |
| 269 | A, 31 |
| 270 | A, 49 |
| 271 | A, 22 |
| 272 | A, 28 |
| 273 | A, 5 |
| 274 | A, 916 |
| 275 | A, 919 |
| 276 | A, 1 |
| 277 | A, 10 |
| 278 | A, 1 |
| 279 | A, 3 |
| 280 | A, 2 |
| 281 | A, 4 |
| 283 | A, 6 |
| 284 | A, 18 |
| 285 | A, 17 |
| 286 | A, 23 |
| 287 | A, 22 |
| 288 | A, 16 |
| 289 | A, 14 |
| 290 | A, 21 |
| 291 | A, 8 |
| 292 | A, 10 |
| 293 | A, 9 |
| 294 | A, 7 |
| 295 | A, 7 |
| 296 | A, 12 |
| 297 | A, 19 |
| 298 | A, 14 |
| 299 | A, 7 |
| 300 | A, 1 |
| 301 | A, 14 |
| 302 | A, 4 |
| 303 | A, 10 |
| 304 | A, 3 |
| 305 | A, 15 |
| 306 | A, 11 |
| 307 | A, 27 |
| 308 | A, 1 |
| 309 | A, 17 |
| 310 | A, 3 |
| 312 | A, 32 |
| 313 | A, 4 |
| 314 | A, 374 |
| 315 | A, 65 |
| 316 | A, 31 |
| 317 | A, 34 |
| 318 | A, 15 |
| 319 | A, 11 |
| 320 | A, 25 |
| 321 | A, 13 |
| 322 | A, 96 |
| 323 | A, 19 |
| 324 | A, 4 |
| 325 | A, 15 |
| 326 | A, 4 |
| 327 | A, 9 |
| 328 | A, 3 |
| 329 | A, 3 |
| 330 | A, 3 |
| 331 | A, 21 |
| 332 | A, 18 |
| 332D | A, 10 |
| 333 | A, 1 |
| 334 | A, 9 |
| 335 | A, 4 |
| 336 | A, 28 |
| 337 | A, 14 |
| 339 | A, 9 |
| 340 | A, 3 |
| 341 | A, 2 |
| 342 | A, 4 |
| 343 | A, 5 |
| 344 | A, 7 |
| 345 | A, 19 |
| 346 | A, 79 |
| 347 | A, 9 |
| 348 | A, 6 |
| 349 | A, 2 |
| 350 | A, 13 |
| 351 | A, 8 |
| 352 | A, 14 |
| 353 | A, 16 |
| 354 | A, 6 |
| 355 | A, 47 |
| 356 | A, 13 |
| 357 | A, 24 |
| 357E | A, 19 |
| 358 | A, 29 |
| 359 | B, 3 |
| 360 | B, 14 |
| 361 | B, 26 |
| 362 | A, 19 |
| 363 | A, 7 |
| 364 | A, 12 |
| 365 | A, 21 |
| 366 | A, 32 |
| 367 | A, 41 |
| 368 | A, 36 |
| 369 | A, 196 |
| 370 | A, 18 |
| 371 | A, 1 |
| 372 | A, 2 |
| 373 | A, 2 |
| 374 | A, 111 |
| 375 | A, 21 |
| 376 | A, 7 |
| 376E | A, 3 |
| 377 | A, 35 |
| 378 | A, 43 |
| 379 | A, 3 |
| 380 | A, 1 |
| 381 | A, 6 |
| 382 | A, 5 |
| 383 | A, 25 |
| 384 | A, 57 |
| 385 | A, 21 |
| 386 | A, 6 |
| 387 | A, 29 |
| 388 | A, 3 |
| 389 | A, 0.4 |
| 391 | A, 4 |
| 391D | A, 2 |
| 391E | A, 4 |
| 391F | A, 6 |
| 392 | A, 151 |
| 393 | A, 197 |
| 393E | A, 162 |
| 394 | A, 249 |
| 395 | A, 210 |
| 396 | A, 146 |
| 397 | A, 134 |
| 398 | A, 246 |
| 398F | A, 130 |
| 399 | A, 310 |
| 400 | A, 299 |
| 401 | A, 250 |
| 402 | A, 219 |
| 403 | A, 229 |
| 404 | A, 254 |
| 405 | A, 304 |
| 406 | A, 309 |
| 407 | A, 322 |
| 408 | A, 22 |
| 409 | A, 24 |
| 410 | A, 198 |
| 411 | A, 5 |
| 412 | A, 50 |
| 413 | A, 113 |
| 414 | A, 11 |
| 415 | A, 8 |
| 416 | A, 29 |
| 418 | A, 108 |
| 419 | A, 120 |
| 420 | A, 13 |
| 421 | A, 11 |
| 422 | A, 15 |
| 423 | A, 30 |
| 424 | A, 49 |
| 425 | A, 14 |
| 426 | A, 30 |
| 427 | A, 12 |
| 428 | A, 21 |
| 429 | A, 60 |
| 431 | A, 14 |
| 432 | A, 8 |
| 433 | A, 18 |
| 434 | A, 10 |
| 435 | A, 7 |
| 436 | A, 12 |
| 437 | A, 8 |
| 438 | A, 6 |
| 439 | A, 2 |
| 440 | A, 3 |
| 441 | A, 10 |
| 442 | A, 14 |
| 445 | A, 5 |
| 446 | A, 126 |
| 447 | A, 19 |
| 448 | A, 4 |
| 449 | A, 34 |
| 450 | A, 53 |
| 451 | A, 225 |
| 452 | A, 198 |
| 453 | A, 7 |
| 454 | A, 1 |
| 455 | A, 246 |
| 456 | A, 41 |
| 457 | A, 18 |
| 458 | A, 3 |
| 459 | A, 392 |
| 460 | A, 67 |
| 461 | A, 89 |
| 462 | A, 26 |
| 463 | A, 400 |
| 464 | A, 636 |
| 465 | A, 64 |
| 466 | A, 50 |
| 467 | A, 64 |
| 468 | A, 26 |
| 470 | A, 75 |
| 471 | A, 146 |
| 472 | A, 62 |
| 473 | A, 107 |
| 474 | A, 45 |
| 478 | A, 771 |
| 480 | A, 3 |
| 481 | A, 16 |
| 482 | A, 6 |
| 482 | B, 3 |
| 483 | A, 4 |
| 484 | A, 4 |
| 485 | A, 26 |
| 486 | A, 27 |
| 487 | A, 3 |
| 488 | A, 1 |
| 489 | A, 53 |
| 490 | A, 24 |
| 491 | A, 6 |
| 492 | A, 53 |
| 493 | A, 6 |
| 494 | A, 38 |
| 495 | A, 87 |
| 496 | A, 344 |
| 497 | A, 1 |
| 498 | A, 41 |
| 499 | A, 121 |
| 500 | A, 188 |
| 501 | A, 292 |
| 502 | A, 39 |
| 503 | A, 9 |
| 504 | A, 2 |
| 505 | A, 6 |
| 506 | A, 886 |
| 507 | A, 82 |
| 508 | A, 195 |
| 509 | A, 21 |
| 510 | A, 44 |
| 511 | A, 450 |
| 512 | A, 66 |
| 513 | A, 32 |
| 514 | A, 131 |
| 515 | A, 130 |
| 516 | A, 164 |
| 517 | A, 28 |
| 518 | A, 138 |
| 519 | A, 1046 |
| 520 | A, 3 |
| 522 | A, 17 |
| 523 | A, 118 |
| 524 | A, 362 |
| 525 | A, 26 |
| 526 | A, 190 |
| 527 | A, 148 |
| 528 | A, 139 |
| 530 | A, 11 |
| 531 | A, 18 |
| 532 | A, 6 |
| 533 | A, 728 |
| 534 | A, 8 |
| 535 | A, 58 |
| 536 | A, 7 |
| 537 | A, 2 |
| 538 | A, 20 |
| 539 | A, 0.4 |
| 540 | A, 216 |
| 541 | A, 5 |
| 542 | A, 4 |
| 543 | A, 7 |
| 544 | A, 5 |
| 545 | A, 22 |
| 546 | A, 29 |
| 548 | A, 90 |
| 549 | A, 5 |
| 549 | A, 351 |
| 550 | A, 8 |
| 551 | A, 6 |
| 552 | A, 27 |
| 553 | A, 16 |
| 555 | A, 7 |
| 556 | A, 6 |
| 561 | A, 2 |
| 562 | A, 2 |
| 563 | A, 3 |
| 564 | A, 2 |
| 565 | A, 3 |
| 566 | A, 12 |
| 567 | A, 4 |
| 568 | A, 5 |
| 569 | A, 3 |
| 570 | A, 3 |
| 571 | A, 8 |
| 572 | A, 3 |
| 573 | A, 73 |
| 575 | A, 1 |
| 576 | A, 7 |
| 577 | A, 6 |
| 578 | A, 17 |
| 579 | A, 4 |
| 580 | A, 5 |
| 581 | A, 4 |
| 582 | A, 10 |
| 583 | A, 16 |
| 583 | A, 26 |
| 584 | A, 26 |
| 585 | A, 9 |
| 586 | A, 4 |
| 587 | A, |
| 588 | A, 2 |
| 589 | A, 2 |
| 590 | A, 2 |
| 591 | A, 1 |
| 592 | A, 0.5 |
| 593 | A, 43 |
| 593 | A, 1 |
| 594 | A, 68 |
| 594 | A, 4 |
| 595 | A, 37 |
| 597 | A, 10 |
| 598 | A, 6 |
| 599 | A, 8 |
| 600 | A, 6 |
| 600D | A, 19 |
| 601 | A, 4 |
| 602 | A, 3 |
| 603 | A, 7 |
| 604 | A, 5 |
| 605 | A, 3 |
| 606 | A, 9 |
| 607 | A, 3 |
| 608 | A, 8 |
| 609 | A, 7 |
| 610 | A, 11 |
| 611 | A, 7 |
| 612 | A, 0.6 |
| 613 | A, 3 |
| 614 | A, 3 |
| 615 | A, 50 |
| 616 | A, 6 |
| 617 | A, 24 |
| 618 | A, 9 |
| 619 | A, 11 |
| 621 | A, 6 |
| 622 | A, 3 |
| 623 | A, 4 |
| 624 | A, 7 |
| 625 | A, 6 |
| 626 | A, 8 |
| 627 | A, 10 |
| 628 | A, 1 |
| 629 | A, 5 |
| 630 | A, 2 |
| 631 | A, 3 |
| 632 | A, 8 |
| 633 | A, 6 |
| 634 | A, 18 |
| 635 | A, 7 |
| 636 | A, 5 |
| 637 | A, 12 |
| 638 | A, 8 |
| 639 | A, 4 |
| 640 | A, 8 |
| 641 | A, 0.9 |
| 642 | A, 4 |
| 643 | A, 8 |
| 644 | A, 0.4 |
| 645 | A, 4 |
| 646 | A, 8 |
| 647 | A, 63 |
| 648 | A, 104 |
| 649 | A, 6 |
| 650 | A, 54 |
| 651 | A, 100 |
| 652 | A, 10 |
| 653 | A, 51 |
| 654 | A, 57 |
| 655 | A, 9 |
| 656 | A, 3 |
| 657 | A, 51 |
| 658 | A, 18 |
| 659 | A, 7 |
| 660 | A, 2 |
| 661 | A, 13 |
| 662 | A, 16 |
| 664 | A, 13 |
| 666 | A, 11 |
| 668 | A, 21 |
| 669 | A, 22 |
| 670 | A, 40 |
| 671 | A, 67 |
| 672 | A, 65 |
| 673 | A, 96 |
| 674 | A, 85 |
| 675 | A, 25 |
| 676 | A, 117 |
| 677 | A, 35 |
| 678 | A, 477 |
| 679 | A, 484 |
| 682 | A, 1422 |
| 683 | A, 193 |
| 684 | A, 261 |
| 685 | A, 276 |
| 686 | A, 7 |
| 688 | A, 161 |
| 689 | A, 53 |
| 690 | A, 507 |
| 691 | A, 115 |
| 692 | A, 98 |
| 693 | A, 69 |
| 694 | A, 103 |
| 695 | A, 242 |
| 696 | A, 80 |
| 700 | A, 25 |
| 701 | A, 11 |
| 702 | A, 12 |
| 703 | A, 29 |
| 704 | A, 11 |
| 705 | A, 760 |
| 706 | A, 57 |
| 707 | A, 68 |
| 708 | A, 494 |
| 709 | A, 16 |
| 710 | A, 13 |
| 711 | A, 997 |
| 712 | A, 1297 |
| 713 | A, 59 |
| 714 | A, 7 |
| 715 | A, 22 |
| 716 | A, 23 |
| 717 | A, 39 |
| 718 | A, 26 |
| 719 | A, 5 |
| 720 | A, 5 |
| 721 | A, 10 |
| 722 | A, 114 |
| 723 | A, 267 |
| 724 | A, 15 |
| 725 | A, 26 |
| 726 | A, 61 |
| 727 | A, 161 |
| 728 | A, 81 |
| 729 | A, 111 |
| 730 | A, 26 |
| 731 | A, 6 |
| 732 | A, 25 |
| 733 | A, 23 |
| 734 | A, 10 |
| 735 | A, 21 |
| 736 | A, 48 |
| 737 | A, 10 |
| 738 | A, 115 |
| 739 | A, 98 |
| 740 | A, 241 |
| 741 | A, 3 |
| 742 | A, 135 |
| 743 | A, 51 |
| 744 | A, 8 |
| 745 | A, 366 |
| 746 | A, 784 |
| 747 | A, 13 |
| 748 | A, 318 |
| 750 | A, 41 |
| 751 | A, 37 |
| 752 | A, 79 |
| 753 | B, 12 |
| 754 | B, 163 |
| 755 | B, 21 |
| 756 | B, 52 |
| 757 | B, 89 |
| 758 | B, 143 |
| 759 | B, 887 |
| 760 | A, 23 |
| 761 | A, 7 |
| 763 | A, 10 |
| 764 | A, 62 |
| 765 | A, 200 |
| 766 | A, 679 |
| 767 | A, 24 |
| 768 | A, 46 |
| 769 | A, 2 |
| 770 | A, 7 |
| 771 | A, 17 |
| 772 | A, 72 |
| 773 | A, 1 |
| 774 | A, 1 |
| 775 | A, 9 |
| 776 | A, 5 |
| 777 | A, 44 |
| 778 | A, 21 |
| 779 | A, 33 |
| 780 | A, 25 |
| 781 | A, 5 |
| 784 | A, 20 |
| 785 | A, 7 |
| 786 | A, 6 |
| 787 | A, 77 |
| 788 | A, 434 |
| 789 | A, 17 |
| 790 | A, 30 |
| 791 | A, 15 |
| 792 | A, 29 |
| 795 | A, 88 |
| 796 | A, 785 |
| 797 | A, 13 |
| 798 | A, 21 |
| 799 | A, 3 |
| 800 | A, 17 |
| 801 | A, 26 |
| 802 | A, 237 |
| 803 | A, 2047 |
| 804 | B, 7 |
| 805 | B, 215 |
| 806 | B, 134 |
| 807 | B, 41 |
| 808 | B, 34 |
| 809 | B, 80 |
| 810 | B, 10 |
| 811 | B, 45 |
| 812 | A, 984 |
| 813 | A, 32 |
| 814 | A, 9 |
| 815 | A, 104 |
| 816 | B, 6 |
| 817 | B. 14 |
| 818 | B, 3 |
| 819 | B, 21 |
| 820 | B, 21 |
| 821 | B, 69 |
| 822 | B, 4 |
| 823 | B, 18 |
| 824 | B, 10 |
| 825 | B, 7 |
| 826 | B, 3 |
| 827 | B, 7 |
| 828 | B, 25 |
| 829 | B, 2 |
| 830 | B, 4 |
| 831 | B, 22 |
| 832 | B, 12 |
| 833 | B, 9 |
| 834 | B, 20 |
| 835 | B, 13 |
| 836 | B, 2 |
| 837 | B, 4 |
| 838 | B, 2 |
| 839 | B, 2 |
| 840 | B, 1 |
| 841 | B, 17 |
| 843 | B, 2 |
| 844 | B, 3 |
| 845 | B, 3 |
| 846 | B, 16 |
| 847 | B, 2 |
| 848 | B, 6 |
| 849 | B, 2 |
| 850 | B, 10 |
| 851 | B, 18 |
| 852 | B, 10 |
| 853 | B, 12 |
| 854 | B, 1 |
| 855 | B, 14 |
| 856 | B, 16 |
| 857 | B, 15 |
| 858 | B, 4 |
| 859 | B, 5 |
| 860 | B, 3 |
| 861 | B, 2 |
| 862 | A, 111 |
| 863 | A, 467 |
| 864 | A, 120 |
| 865 | A, 731 |
| 866 | A, 517 |
| 867 | A, 213 |
| 868 | A, 114 |
| 869 | A, 125 |
| 870 | A, 32 |
| 871 | A, 6 |
| 872 | A, 3 |
| 873 | A, 936 |
| 874 | A, 482 |
| 875 | A, 477 |
| 876 | A, 264 |
| 879 | A, 149 |
| 880 | A, 15 |
| 881 | A, 7 |
| 882 | A, 1 |
| 883 | A, 6 |
| 884 | A, 99 |
| 885 | A, 8 |
| 886 | A, 4 |
| 887 | A, 8 |
| 888 | A, 6 |
| 889 | A, 21 |
| 890 | A, 1 |
| 891 | A, 25 |
| 892 | A, 15 |
| 893 | A, 7 |
| 894 | A, 14 |
| 895 | A, 5 |
| 896 | A, 10 |
| 897 | A, 8 |
| 898 | A, 10 |
| 899 | A, 12 |
| 900 | A, 12 |
| 901 | A, 29 |
| 902 | A, 28 |
| 903 | A, 8 |
| 904 | A, 12 |
| 905 | A, 11 |
| 906 | A, 1 |
| 907 | A, 4 |
| 908 | A, 3 |
| 909 | A, 14 |
| 910 | A, 59 |
| 911 | A, 70 |
| 912 | A, 214 |
| 913 | A, 3 |
| 914 | A, 3 |
| 915 | A, 10 |
| 916 | B, 56 |
| 917 | B, 3 |
| 918 | B, 2 |
| 919 | B, 5 |
| 920 | B, 2 |
| 921 | B, 7 |
| 922 | B, 0.6 |
| 923 | B, 1 |
| 924 | B, 0.9 |
| 925 | B, 0.4 |
| 926 | B, 0.8 |
| 927 | B, 0.1 |
| 928 | B, 0.1 |
| 929 | B, 1 |
| 930 | B, 1 |
| 931 | B, 0.5 |
| 932 | B, 3 |
| 933 | B, 0.8 |
| 934 | B, 0.5 |
| 935 | B, 0.2 |
| 936 | B, 2 |
| 937 | B, 1 |
| 938 | B, 2 |
| 939 | B, 0.9 |
| 940 | B, 3 |
| 941 | B, 1 |
| 942 | B, 4 |
| 943 | B, 2 |
| 944 | B, 1 |
| 945 | B, 4 |
| 946 | B, 12 |
| 947 | B, 1 |
| 948 | B, 2 |
| 949 | B, 0.8 |
| 950 | B, 0.6 |
| 951 | B, 3 |
| 952 | B, 7 |
| 953 | B, 7 |
| 954 | B, 4 |
| 955 | B, 17 |
| 956 | B, 2 |
| 957 | B, 0.6 |
| 958 | B, 2 |
| 959 | B, 2 |
| 960 | B, 1 |
| 961 | B, 5 |
| 962 | B, 5 |
| 963 | B, 3 |
| 964 | B, 46 |
| 965 | B, 3 |
| 966 | B, 8 |
| 967 | B, 0.9 |
| 968 | B, 2 |
| 969 | B, 4 |
| 970 | B, 5 |
| 971 | B, 1 |
| 972 | B, 2 |
| 973 | B, 4 |
| 974 | B, 1 |
| 975 | B, 7 |
| 976 | B, 1 |
| 977 | B, 1 |
| 978 | B, 3 |
| 979 | B, 4 |
| 980 | B, 1 |
| 981 | B, 1 |
| 982 | B, 6 |
| 983 | B, 2 |
| 984 | B, 14 |
| 985 | B, 4 |
| 986 | B, 37 |
| 986E | B, 0.9 |
| 987 | B, 1 |
| 988 | B, 3 |
| 989 | B, 0.9 |
| 990 | B, 1 |
| 991 | B, 0.8 |
| 992 | B, 10 |
| 993 | B, 2 |
| 994 | B, 0.6 |
| 995 | B, 1 |
| 996 | B, 1.1 |
| 997 | B, 0.6 |
| 998 | B, 1.1 |
| 999 | B, 0.7 |
| 1000 | B, 0.7 |
| 1001 | B, 0.6 |
| 1002 | B, 0.3 |
| 1003 | B, 0.8 |
| 1004 | B, 0.9 |
| 1005 | B, 0.5 |
| 1006 | B, 0.8 |
| 1007 | B, 1 |
| 1008 | B, 1 |
| 1009 | B, 1.9 |
| 1010 | B, 2.1 |
| 1011 | B, 2.2 |
| 1012 | B, 1.1 |
| 1013 | B, 1.7 |
| 1014 | |
| 1015 | |
| 1016 | B, 1 |
| 1017 | B, 1.6 |
| 1018 | B, 0.8 |
| 1019 | B, 0.5 |
| 1020 | B, 1.1 |
| 1021 | B, 0.7 |
| 1022 | B, 1.3 |
| 1023 | B, 0.8 |
| 1024 | B, 0.8 |
| 1025 | |
| 1026 | B, 2.2 |
| 1027 | |
| 1028 | B, 1.7 |
| 1029 | B, 0.1 |
| 1030 | B, 0.9 |
| 1031 | B, 1.1 |
| 1032 | B, 1.2 |
| 1033 | B, 1 |
| 1034 | |
| 1035 | B, 0.8 |
| 1036 | B, 0.1 |
| 1037 | B, 1.2 |
| 1038 | B, 0.9 |
| 1039 | B, 0.9 |
| 1040 | B, 1.7 |
| 1041 | B, 1.7 |
| 1042 | |
| 1043 | B, 2.1 |
| 1044 | B, 2.8 |
| 1045 | B, 0.5 |
| 1046 | B, 16 |
| 1047 | B, 9.9 |
| 1048 | B, 3.1 |
| 1049 | B, 1.9 |
| 1050 | B, 0.6 |
| 1051 | B, 2.2 |
| 1052 | B, 0.9 |
| 1053 | B, 0.8 |
| 1054 | |
| 1055 | B, 0.5 |
| 1056 | B, 0.7 |
| 1057 | B, 1.7 |
| 1058 | B, 1.1 |

### V. Pharmaceutical Compositions, Formulations and Combinations

The compounds of this invention can be administered in such oral dosage forms as tablets, capsules (each of which includes sustained release or timed-release formulations), pills, powders, granules, elixirs, tinctures, suspensions, syrups, and emulsions. They may also be administered in intravenous (bolus or infusion), intraperitoneal, subcutaneous, or intramuscular form, using dosage forms well known to those of ordinary skill in the pharmaceutical arts. They can be administered alone, but generally will be administered with a pharmaceutical carrier selected on the basis of the chosen route of administration and standard pharmaceutical practice.

The term “pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Pharmaceutically acceptable carriers are formulated according to a number of factors well within the purview of those of ordinary skill in the art. These include, without limitation: the type and nature of the active agent being formulated; the patient to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 18th Edition (1990).

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, and most preferably between about 0.1 to about 20 mg/kg/day. Intravenously, the most preferred doses will range from about 0.001 to about 10 mg/kg/minute during a constant rate infusion. Compounds of this invention may be administered in a single daily dose, or the total daily dosage may be administered in divided doses of two, three, or four times daily.

Compounds of this invention can also be administered by parenteral administration (e.g., intra-venous, intra-arterial, intramuscularly, or subcutaneously. When administered intra-venous or intra-arterial, the dose can be given continuously or intermittent. Furthermore, formulation can be developed for intramuscularly and subcutaneous delivery that ensure a gradual release of the active pharmaceutical ingredient.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 1000 milligrams of active ingredient per dosage unit. In these pharmaceutical compositions the active ingredient will ordinarily be present in an amount of about 0.1-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

The compounds of the present invention can be administered alone or in combination with one or more additional therapeutic agents. By “administered in combination” or “combination therapy” it is meant that the compound of the present invention and one or more additional therapeutic agents are administered concurrently to the mammal being treated. When administered in combination, each component may be administered at the same time or sequentially in any order at different points in time. Thus, each component may be administered separately but sufficiently closely in time so as to provide the desired therapeutic effect.

The compounds of the present invention are also useful as standard or reference compounds, for example, as a quality standard or control, in tests or assays involving the inhibition of SGK1. Such compounds may be provided in a commercial kit, for example, for use in pharmaceutical research involving SGK1. For example, a compound of the present invention could be used as a reference in an assay to compare its known activity to a compound with an unknown activity. This would ensure the experimentor that the assay was being performed properly and provide a basis for comparison, especially if the test compound was a derivative of the reference compound. When developing new assays or protocols, compounds according to the present invention could be used to test their effectiveness.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises, a first therapeutic agent, comprising: a compound of the present invention or a pharmaceutically acceptable salt form thereof; and, (c) a package insert stating that the pharmaceutical composition can be used for the treatment of a cardiovascular and/or inflammatory disorder (as defined previously). In another embodiment, the package insert states that the pharmaceutical composition can be used in combination (as defined previously) with a second therapeutic agent to treat cardiovascular and/or inflammatory disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

Other features of the invention will become apparent in the course of the following descriptions of exemplary embodiments that are given for illustration of the invention and are not intended to be limiting thereof. The following Examples have been prepared, isolated and characterized using the methods disclosed herein.

### VI. General Synthesis Including Schemes

The compounds of the present invention may be synthesized by many methods available to those skilled in the art of organic chemistry (Maffrand, J. P. et al., Heterocycles, 16(1):35-37 (1981)). General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds.

Examples of compounds of the present invention prepared by methods described in the general schemes are given in the intermediates and examples section set out hereinafter. Preparation of homochiral examples may be carried out by techniques known to one skilled in the art. For example, homochiral compounds may be prepared by separation of racemic products by chiral phase preparative HPLC. Alternatively, the example compounds may be prepared by methods known to give enantiomerically enriched products. These include, but are not limited to, the incorporation of chiral auxiliary functionalities into racemic intermediates which serve to control the diastereoselectivity of transformations, providing enantio-enriched products upon cleavage of the chiral auxiliary.

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, 4th Edition. Wiley-Interscience (2006)).

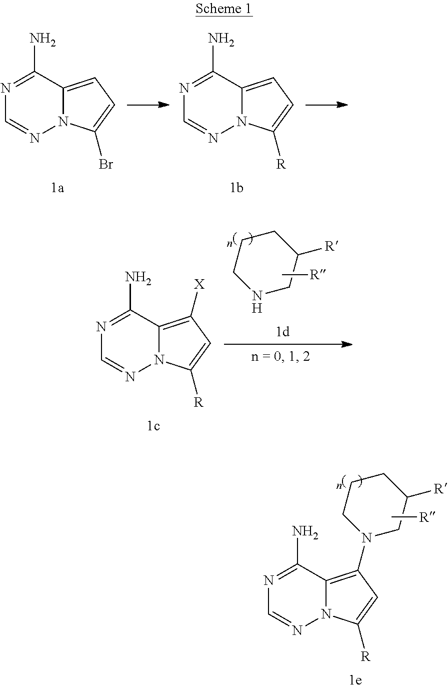

Scheme 1 describes the synthesis of compound 1e from the coupling of compound 1d with compound 1c via photocatalyzed N-arylation methods (Corcoran, Emily B., et al. “Aryl amination using ligand-free Ni (II) salts and photoredox catalysis.” Science 353.6296 (2016): 279-283). Alternatively, other methods such as Pd catalysis may be utilized for N-arylation. Compound 1e may be further elaborated on the R, R′ and R″ substituents, as can any intermediate in this scheme. Compound 1c is prepared from intermediate 1b via halogenation with NIS or NBS, or other halogenating reagents. Intermediate 1b is prepared from intermediate 1a via cross-coupling with an appropriate coupling partner using PdCl₂(dppf) or other suitable reagent.

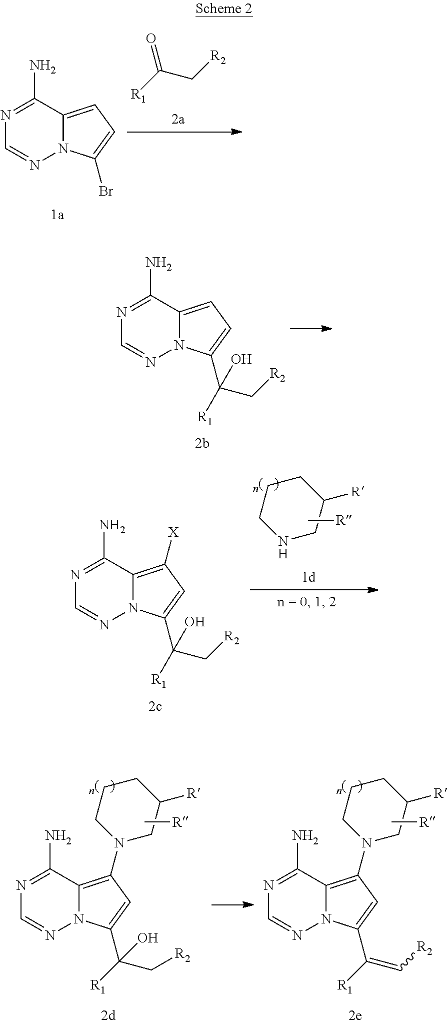

Scheme 2 describes the synthesis of compound 2e from compound 2d via dehydration with (methoxycarbonylsulfamoyl)triethylammonium hydroxide (Burgess reagent). Other dehydration methods may be utilized, such as reaction with methanesulfonyl chloride, followed by treatment with a base such as TEA. Compound 2e may be further elaborated on the R₁, R₂, R′ and R″ substitutents, as can any intermediate in this scheme. Compound 2d is prepared via the coupling of amine compound 1d and compound 2c via photocatalyzed N-arylation methods. Alternatively, other methods such as Pd catalysis may be utilized for N-arylation. Compound 2c is prepared via halogenation of compound 2b with reagents such as NBS or NIS. Compound 2b is prepared from compound 1a via persilylation of the amine with TMS-Cl, followed by halogen-metal exchange with i-PrMgCl or an alkyl lithium reagent, followed by reaction with the ketone compound 2a. The order of the dehydration and N-arylation steps may be reversed.

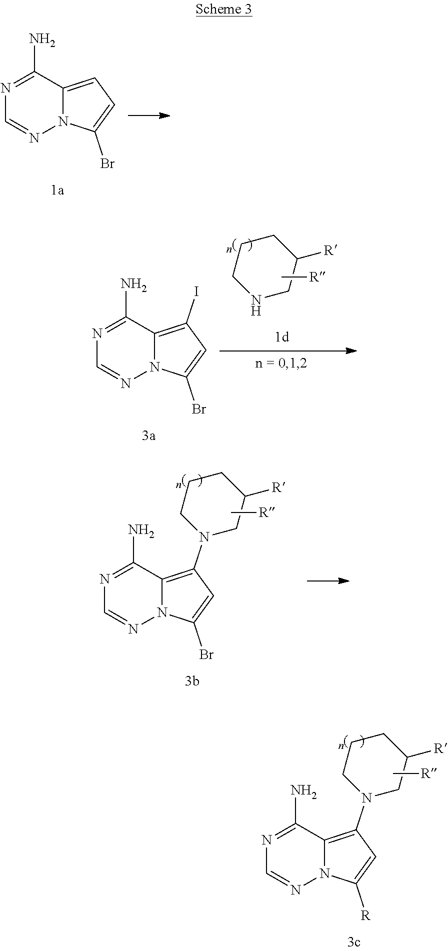

Scheme 3 describes the synthesis of compound 3c from the compound 3b via cross coupling (i.e., Suzuki coupling, etc.) using PdCl₂(dppf) or other appropriate reagent. Compound 3b is prepared via the coupling of amine compound 1d and compound 3a via photocatalyzed N-arylation methods. Alternatively, other methods such as Pd catalysis may be utilized for N-arylation. Compound 3a is prepared via iodination of compound 1a with NIS. Compound 3c may be further elaborated on the R, R′ and R″ substituents, as can any intermediate in this scheme.

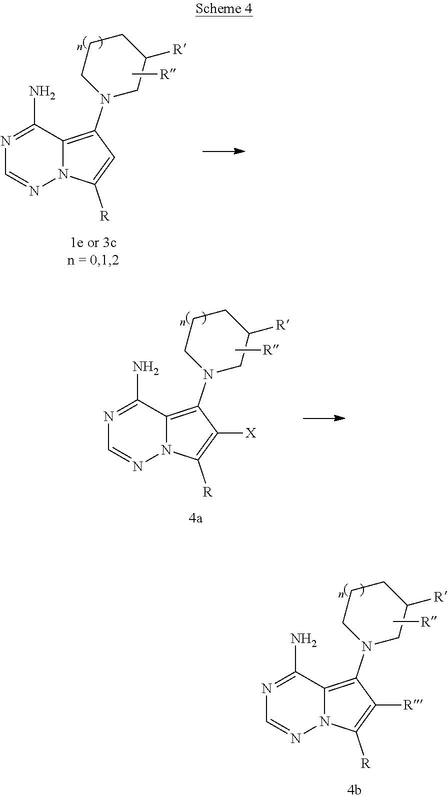

Scheme 4 describes the synthesis of halo compound 4a (X=halogen) from either compound 1e or compound 3c via halogenation using a reagent such as N-bromosuccinimide, N-chlorosuccinimide, or other appropriate reagent. Compound 4a (X ═Cl or Br) can be further elaborated to compound 4b via metal-mediated coupling with reagents such as Zn(CN)₂/Pd(PPh₃)₄, trimethylboroxine/PdCl₂(dppf), potassium cyclopropyltrifluoroborate/PdCl₂(dppf). Compound 4b may also be prepared via metal/halogen exchange of compound 4a with i-PrMgCl or BuLi, followed by reaction with an appropriate electrophile. Compound 4b may be further elaborated on the R, R′, R′ and R′″ substituents, as can any intermediate in this scheme.

Purification of intermediates and final products was carried out via either normal or reverse phase chromatography. Normal phase chromatography was carried out using prepacked SiO₂ cartridges eluting with either gradients of hexanes and EtOAc or DCM and MeOH unless otherwise indicated. Reverse phase preparative HPLC was carried out using C18 columns eluting with gradients of Solvent A (90% H₂O, 10% MeOH, 0.1% TFA) and Solvent B (10% H₂O, 90% MeOH, 0.1% TFA, UV 220 nm) or with gradients of Solvent A (90% H₂O, 10% ACN, 0.1% TFA) and Solvent B (10% H₂O, 90% ACN, 0.1% TFA, UV 220 nm) or with gradients of Solvent A (98% H₂O, 2% ACN, 0.05% TFA) and Solvent B (98% ACN, 2% H₂O, 0.05% TFA, UV 220 nm) (or) Sunfire Prep C18 OBD 5u 30×100 mm, 25 min gradient from 0-100% B. A=H₂O/ACN/TFA 90:10:0.1. B=ACN/H₂O/TFA 90:10:0.1 (or) Waters XBridge C18, 19×200 mm, 5-μm particles; Guard Column: Waters XBridge C18, 19×10 mm, 5-μm particles; Solvent A: water with 20-mM ammonium acetate; Solvent B: 95:5 acetonitrile:water with 20-mM ammonium acetate; Gradient: 25-65% B over 20 minutes, then a 5-minute hold at 100% B; Flow: 20 mL/min.

Unless otherwise stated, analysis of final products was carried out by reverse phase analytical HPLC.

### Analytical HPLC Methods

Method A: Column: Waters Acquity UPLC BEH C18, 2.1×50 mm, 1.7-μm particles; Mobile Phase A: 5:95 acetonitrile/water with 10 mM ammonium acetate; Mobile Phase B: 95:5 acetonitrile/water with 10 mM ammonium acetate; Temperature: 50° C.; Gradient: 0-100% B over 3 minutes, then a 0.75-minute hold at 100% B; Flow: 1.11 mL/min. UV 220 nm.

Method B: Column: Waters Acquity UPLC BEH C18, 2.1×50 mm, 1.7-μm particles; Mobile Phase A: 5:95 acetonitrile/water with 0.1% TFA; Mobile Phase B: 95:5 acetonitrile/water with 0.1% TFA; Temperature: 50° C.; Gradient: 0-100% B over 3 minutes, then a 0.75-minute hold at 100% B; Flow: 1.11 mL/min. UV 220 nm.

Method C: Column: SunFire C18, 3.0×150 mm, 3.5-μm particles; Mobile Phase A: 5:95 acetonitrile/water with 0.1% TFA; Mobile Phase B: 95:5 acetonitrile/water with 0.1% TFA; Temperature: 25° C.; Gradient: 10-100% B over 10 minutes, then a 5-minute hold at 100% B: Flow: 1.0 mL/min. UV 220 nm.

Method D: Column: XBridge Phenyl, 3.0×150 mm, 3.5-μm particles; Mobile Phase A: 5:95 acetonitrile/water with 0.1% TFA; Mobile Phase B: 95:5 acetonitrile/water with 0.1% TFA; Temperature: 25° C.; Gradient: 10-100% B over 10 minutes, then a 5-minute hold at 100% B; Flow: 1.0 mL/min. UV 220 nm.

Method E: Column: XBridge BEH XP C18 (50×2.1 mm, 2.5 μm); Mobile phase A: 10 mM NH₄OAc/Acetonitrile (95:5); Mobile phase B: 10 mM NH₄OAc/Acetonitrile (5:95); Gradient=0-100% B over 3 minutes; Temperature: 50° C.; Flow rate: 1.1 mL/min; Detection: UV at 220 nm.

Method F: Column: XBridge BEH XP C18 (50×2.1 mm, 2.5 μm); Mobile phase A: 0.1% TFA in water, Acetonitrile (95:5); Mobile phase B: 0.1% TFA in water/Acetonitrile (5:95); Gradient=0-100% B over 3 minutes; Temperature: 50° C.; Flow rate: 1.1 mL/min; Detection: UV at 220 nm.

Method G: Column: Kinetex EVO-C18 (4.6×100 mm, 2.6 μm). Buffer: 0.05% TFA in water, Mobile Phase C: Buffer/Acetonitrile (95:5), Mobile Phase D: Acetonitrile/Buffer (95:5), Gradient: 10% B to 60% B over 9 min, Flow: 1.0 mL/min; 60% B to 100% B up to 3.5 min, Flow: 1.5 mL/min; hold until 15 min; 2.5 min, UV (300 nm).

Method H: Column: Kinetex Biphenyl-C18 (4.6×100 mm, 2.6 μm); Buffer: 0.05% TFA in water, Mobile Phase C: Buffer/Acetonitrile (95:5), Mobile Phase D: Acetonitrile/Buffer (95:5), Gradient: 10% B to 60% B over 9 min, Flow: 1.0 mL/min; 60% B to 100% B up to 3.5 min, Flow: 1.5 mL/min; hold until 15 min; 2.5 min, UV (300 nm).

Method I: Column: Kinetex EVO-C18 (4.6×100 mm, 2.6 μm); Buffer: 0.05% TFA in water, Mobile Phase C: Buffer/Acetonitrile (95:5), Mobile Phase D: Acetonitrile/Buffer (95:5), Gradient: 0% B to 30% B over 9 min, Flow: 1.0 mL/min; 30% B to 100% B up to 3.5 min, Flow: 1.5 mL/min; hold until 13 min; 2.5 min, UV (300 nm), Temparature−60° C.

Method J: Column: Kinetex Biphenyl-C18 (4.6×100 mm, 2.6 μm); Buffer: 0.05% TFA in water, Mobile Phase C: Buffer/Acetonitrile (95:5), Mobile Phase D: Acetonitrile/Buffer (95:5), Gradient: 0% B to 30% B over 9 min, Flow: 1.0 mL/min; 30% B to 100% B up to 3.5 min, Flow: 1.5 mL/min; hold till 13 min; 0.5 min, UV (300 nm), Temparature−60° C.

Method K: Column: Xbridge Phenyl (4.6×150 mm, 3 μm); Buffer: 0.1% TFA in water, Mobile Phase C: Buffer/Acetonitrile (95:5). Mobile Phase D: Acetonitrile/Buffer (95:5), Gradient: 5% B to 40% B over 15 min, Flow: 1.0 mL/min; 40% B to 100% B up to 22 min, Flow: 1.0 mL/min; hold until 22 min; 6 min, UV (300 nm), Temparature−60° C.

Method L: Column: Waters XBridge C18, 150 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 12% B, 12-32% B over 25 minutes, then a 5-minute hold at 100% B; Flow Rate: 20 mL/min

Method M: Column: Waters XBridge C18, 150 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 15% B, 15-30% B over 20 minutes, then a 5-minute hold at 100% B: Flow Rate: 20 mL/min.

Method N: Column: Waters XBridge C18, 150 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 12% B, 12-45% B over 25 minutes, then a 5-minute hold at 100% B; Flow Rate: 20 mL/min

Method O: Column: Ascentis Express C18 (50 mm×2.1 mm), 2.7 μm; Mobile Phase A: 5:95 acetonitrile: water with 10 mM NH₄OAc; Mobile Phase B: 95:5 acetonitrile: water with 10 mM NH₄OAc; Gradient: 0-100% B over 30 minutes; Flow Rate: 20 mL/min

Method P: Waters XBridge C18, 150 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 15% B, 15-35% B over 15 minutes, then a 5-minute hold at 100% B; Flow Rate: 20 mL/min.

### Intermediate 1.

### 6-(4,4,5,5-tetramethyl-1,3,2-dioxaborolan-2-yl)-[1,2,4]triazolo[1,5-a]pyrimidine

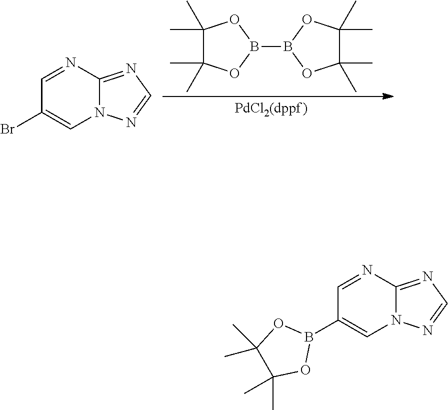

To a vial containing 6-bromo-[1,2,4]triazolo[1,5-a]pyrimidine (200 mg, 1.005 mmol), bis(pinacolato)diboron (306 mg, 1.206 mmol) and potassium acetate (296 mg, 3.01 mmol), was added Dioxane (5 mL). The mixture was degassed (evacuated and flushed with N2, 3×), then PdCl₂(dppf)-CH₂C₂ adduct (82 mg, 0.100 mmol) was added. The mixture was degassed (3×), then the vial was sealed and heated at 90° C. for 3.75 h. The mixture was diluted with EtOAc, then filtered through 1″ Celite, rinsing with EtOAc. the filtrate was concentrated to afford Intermediate 1 (43.5% purity, 570 mg, 100% yield) as a brown solid. The mixture was used without further purification. MS: [M+H]⁺=165.1 (boronic acid)

### Intermediate 2.

### 1-(2-fluoro-4-(4,4,5,5-tetramethyl-1,3,2-dioxaborolan-2-yl)phenyl)-1H-tetrazole

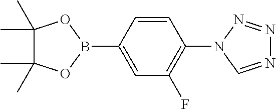 [SMILES: CC1(C)OB(c2ccc(-n3cnnn3)c(F)c2)OC1(C)C]

According to the procedure for the preparation of Intermediate 1, starting with 1-(4-bromo-2-fluorophenyl)-1H-tetrazole afforded after flash chromatographic purification Intermediate 2. MS: [M+H]⁺=209.0; ¹H NMR (500 MHz, CDCl₃) δ 9.14 (d, J=2.8 Hz, 1H), 8.03-7.95 (m, 1H), 7.83-7.73 (m, 2H), 1.37 (s, 12H); ¹⁹F NMR (471 MHz, CDCl₃) δ −124.01 (s, 1F)

### Intermediate 3.

### 1-(4-(4,4,5,5-tetramethyl-1,3,2-dioxaborolan-2-yl)phenyl)-1H-tetrazole

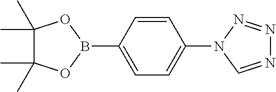 [SMILES: CC1(C)OB(c2ccc(-n3cnnn3)cc2)OC1(C)C]

According to the procedure for the preparation of Intermediate 1, starting with 1-(4-bromophenyl)-1H-tetrazole afforded crude Intermediate 3 (~51% purity), which was used without further purification. MS: [M+H]⁺=272.9

### Intermediate 4.

### 7-(4,4,5,5-tetramethyl-1,3,2-dioxaborolan-2-yl)tetrazolo[1,5-a]pyridine

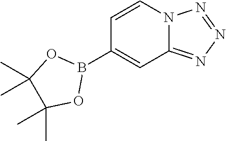 [SMILES: CC1(C)OB(c2ccn3nnnc3c2)OC1(C)C]

According to the procedure for the preparation of Intermediate 1, starting with 7-bromotetrazolo[1,5-a]pyridine afforded crude Intermediate 4 (49% purity), which was used without further purification. MS: [M+H]⁺=165.1 (boronic acid)

### Intermediate 5.

### 6-(4,4,5,5-tetramethyl-1,3,2-dioxaborolan-2-yl)-[1,2,4]triazolo[4,3-a]pyridine

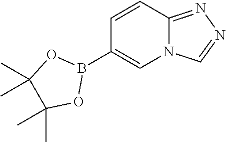 [SMILES: CC1(C)OB(c2ccc3nncn3c2)OC1(C)C]

According to the procedure for the preparation of Intermediate 1, starting with 6-bromo-[1,2,4]triazolo[4,3-a]pyridine afforded crude Intermediate 5 (48.5% purity), which was used without further purification. MS: [M+H]⁺=164.0 (boronic acid)

### Intermediate 6.

### 3-methyl-6-(4,4,5,5-tetramethyl-1,3,2-dioxaborolan-2-yl)-[1,2,4]triazolo[4,3-a]pyridine

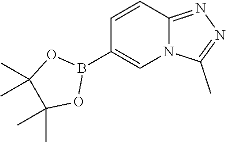 [SMILES: Cc1nnc2ccc(B3OC(C)(C)C(C)(C)O3)cn12]

According to the procedure for the preparation of Intermediate 1, starting with 6-bromo-3-methyl-[1,2,4]triazolo[4,3-a]pyridine afforded crude Intermediate 6 (52.5% purity), which was used without further purification. MS: [M+H]⁺=178.0 (boronic acid)

### Intermediate 7.

### 1-(5-(4,4,5,5-tetramethyl-1,3,2-dioxaborolan-2-yl)pyridin-2-yl)-1H-1,2,4-triazole-3-carbonitrile

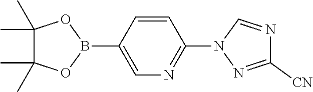 [SMILES: CC1(C)OB(c2ccc(-n3cnc(C#N)n3)nc2)OC1(C)C]

According to the procedure for the preparation of Intermediate 1, starting with 1-(5-bromopyridin-2-yl)-1H-1,2,4-triazole-3-carbonitrile afforded crude Intermediate 7 (55% purity), which was used without further purification. MS: [M+H]⁺=216.0 (boronic acid)

### Intermediate 8.

### 5-(4,4,5,5-tetramethyl-1,3,2-dioxaborolan-2-yl)-2-(1H-1,2,4-triazol-1-yl)pyridine

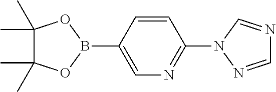 [SMILES: CC1(C)OB(c2ccc(-n3cncn3)nc2)OC1(C)C]

According to the procedure for the preparation of Intermediate 1, starting with 5-bromo-2-(1H-1,2,4-triazol-1-yl)pyridine afforded crude Intermediate 8 (67% purity), which was used without further purification. MS: [M+H]⁺=216.0 (boronic acid)

### Intermediate 9. Preparation of 5-bromo-1-ethylpyridin-2(1H)-one (9A) and 5-bromo-2-ethoxypyridine (9B)

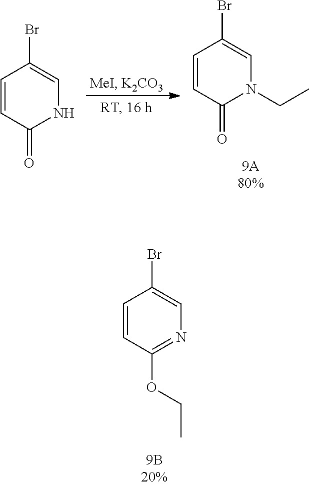

To a stirred solution of 5-bromopyridin-2(1H)-one (0.5 g, 2.87 mmol)) in DMF (5 mL), ethyl iodide (0.522 mL, 6.47 mmol) and K₂CO₃ (0.894 g, 6.47 mmol) were added. Reaction mixture was allowed to stir at 80° C. for 12 h. After cooling, mixture was filtered through Celite and filtrate was concentrated. Residue obtained was purified by silica-gel chromatography with ethyl acetate and pet-ether mixture with a gradient of 0-100% which afforded Intermediate 9A (320 mg, 80% yield) as a brown solid; LCMS Method E: RT=1.08 min, [M+H]⁺=202.9 and Intermediate 9B (100 mg, 20% yield) as colorless liquid; RT=1.61 min, MS [M+H]⁺=202.9.

### Intermediate 10. Preparation of 1-ethyl-5-(trimethylstannyl)pyridin-2(1H)-one

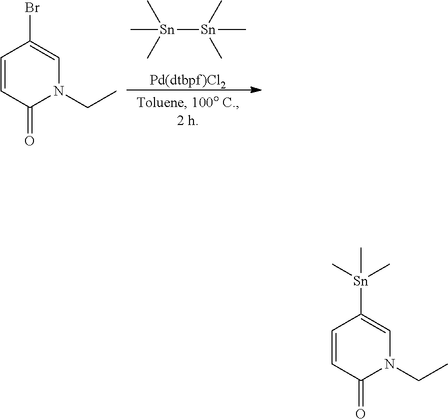

Intermediate 9A (0.33 g, 1.633 mmol) was taken in toluene (5 mL) and purged with N₂ for 5 minutes. To this mixture, hexamethylditin (0.745 mL, 3.59 mmol) and [1,1′-bis(di-tert-butylphosphino)ferrocene]dichloropalladium(II) (0.053 g, 0.082 mmol) were added, sealed the vial, and stirred for 2 h at 100° C. Reaction mixture was filtered and the filtrate was concentrated to afford Intermediate 10 (320 mg, 86% yield) as an off-white solid. LCMS Method E: MS [M+H]⁺=286.0.

### Intermediate 11. Preparation of 5-bromo-1-(2,2-difluoroethyl)pyridin-2(1H)-one

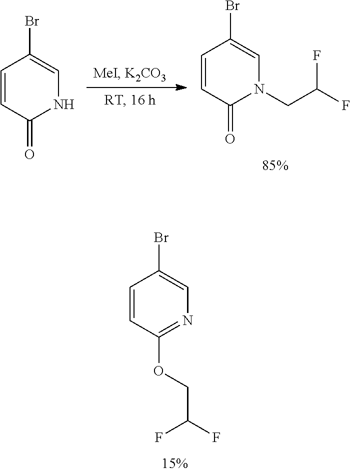 [SMILES: O=c1ccc(Br)c[nH]1 ; O=c1ccc(Br)cn1CC(F)F ; FC(F)COc1ccc(Br)cn1]

Analogous to the procedure for the preparation of Intermediate 9A & 9B, reaction of 5-bromopyridin-2(1H)-one (700 mg, 3.93 mmol) and 2,2-difluoroethyl trifluoromethanesulfonate yielded Intermediate 11A (480 mg, 70% yield) as a brown liquid; MS: RT=0.94 min, [M+H]⁺=237.9 and Intermediate 11B (220 mg, 25% yield) as an off-white solid; LCMS Method E: RT=1.70 min, [M+H]⁺=237.9.

### Intermediate 12. 5-(4,4,5,5-tetramethyl-1,3,2-dioxaborolan-2-yl)pyrimidine

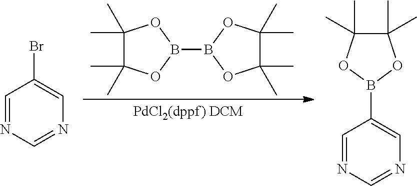

To a vial containing 5-bromopyrimidine (200 mg, 1.01 mmol), bis(pinacolato)diboron (306 mg, 1.21 mmol) and potassium acetate (296 mg, 3.01 mmol), was added dioxane (5 mL). The mixture was degassed with N2 and PdCl₂(dppf)-CH₂Cl₂ adduct (82 mg, 0.10 mmol) was added. The vial was sealed and heated at 90° C. for 4 h. After cooling, mixture was diluted with EtOAc and passed through Celite. Filtrate was concentrated to afford Intermediate 12 (570 mg) as a crude brown solid which was taken up for next step without further purification. LCMS Method E: MS [M+H]⁺=207.1 (boronic acid).

### Intermediate 13. 2-(5-(4,4,5,5-tetramethyl-1,3,2-dioxaborolan-2-yl)pyrimidin-2-yl)propan-2-ol

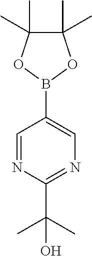

Analogous to the procedure for the preparation of Intermediate 12, 2-(5-bromopyrimidin-2-yl)propan-2-ol (350 mg, 1.52 mmol) afforded Intermediate 13 (410 mg, 85% yield) as a brown color semi-solid. MS [M+H]⁺=265.2.

### Intermediate 14. 2-(2,2-difluoroethoxy)-5-(4,4,5,5-tetramethyl-1,3,2-dioxaborolan-2-yl)pyridine

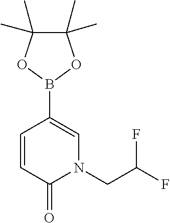 [SMILES: CC1(C)OB(c2ccc(=O)n(CC(F)F)c2)OC1(C)C]

Analogous to the procedure for the preparation of Intermediate 12, 5,5-bromo-2-(2,2-difluoroethoxy)pyridine (350 mg, 1.48 mmol) afforded Intermediate 14 (380 mg, 80% yield) as a brown color semi-solid. MS [M+H]⁺=286.2.

### Intermediate 15. 2-(2,2-difluoroethoxy)-5-(4,4,5,5-tetramethyl-1,3,2-dioxaborolan-2-yl)pyridine

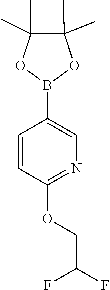 [SMILES: CC1(C)OB(c2ccc(OCC(F)F)nc2)OC1(C)C]

Analogous to the procedure for the preparation of Intermediate 12, 5-bromo-1-(2,2-difluoroethyl)pyridin-2(1H)-one (150 mg, 0.632 mmol) afforded Intermediate 15 (180 mg, 90% yield) as a brown color semi-solid. MS [M+H]⁺=286.2.

### Intermediate 16. 3-fluoro-5-(trimethylstannyl)picolinonitrile

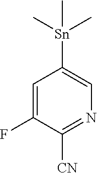

Analogous to the procedure for the preparation of Intermediate 10, 5-bromo-3-fluoropicolinonitrile (300 mg, 1.49 mmol) afforded Intermediate 16 (380 mg, 85% yield) as a brown color semi-solid. MS [M+H]⁺=286.9.

### Intermediate 17. 3-(trifluoromethyl)-5-(trimethylstannyl)picolinonitrile

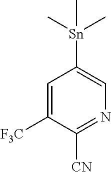 [SMILES: [CH3][Sn]([CH3])([CH3])[c]1cnc(C#N)c(C(F)(F)F)c1]

Analogous to the procedure for the preparation of Intermediate 10, 5-bromo-3-trifluoropicolinonitrile (300 mg, 1.23 mmol) afforded Intermediate 17 (360 mg, 79% yield) as a brown color semi-solid. MS [M+H]⁺=336.9.

### Intermediate 18. 5-(4,4,5,5-tetramethyl-1,3,2-dioxaborolan-2-yl)-1,2-dihydropyridine-2-carbonitrile

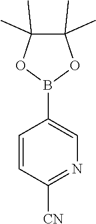

Following the procedure for the preparation of Intermediate 12, 5-bromo-1,2-dihydropyridine-2-carbonitrile (250 mg, 1.24 mmol) afforded Intermediate 18 (250 mg, 75% yield) as a brown color semi-solid. MS [M+H]⁺=233.2.

### Intermediate 19. 5-(4,4,5,5-tetramethyl-1,3,2-dioxaborolan-2-yl)pyridin-2(1H)-one

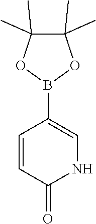

Analogous to the procedure for the preparation of Intermediate 12, 5-bromopyridin-2(1H)-one (300 mg, 1.13 mmol) afforded Intermediate 19 (320 mg, 75% yield) as a brown color semi-solid. MS [M+H]⁺=222.2.

### Intermediate 20. 3-methyl-5-(4,4,5,5-tetramethyl-1,3,2-dioxaborolan-2-yl)picolinonitrile

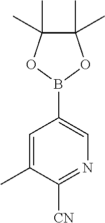

Analogous to the procedure for the preparation of Intermediate 12, 5-bromo-3-methylpicolinonitrile (300 mg, 1.12 mmol) afforded Intermediate 20 (350 mg, 80% yield) as a brown color semi-solid. MS [M+H]⁺=245.2.

### Intermediate 21. 3-fluoro-5-(4,4,5,5-tetramethyl-1,3,2-dioxaborolan-2-yl)picolinonitrile

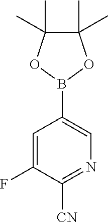 [SMILES: CC1(C)OB(c2cnc(C#N)c(F)c2)OC1(C)C]

Analogous to the procedure for the preparation of Intermediate 12, 5-bromo-3-fluoro-picolinonitrile (300 mg, 1.14 mmol) afforded Intermediate 21 (330 mg, 75% yield) as a brown color semi-solid. MS [M+H]⁺=248.5.

### Intermediate 22. 5-(4,4,5,5-tetramethyl-1,3,2-dioxaborolan-2-yl)-3-(trifluoromethyl)picolinonitrile

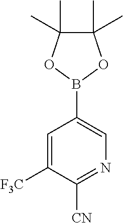 [SMILES: CC1(C)OB(c2cnc(C#N)c(C(F)(F)F)c2)OC1(C)C]

Analogous to the procedure for the preparation of Intermediate 12, 5-bromo-3-(trifluoromethyl)picolinonitrile (300 mg, 1.25 mmol) afforded Intermediate 22 (360 mg, 79% yield) as a brown color semi-solid. MS [M+H]⁺=299.1.

### Intermediate 23. 5-(4,4,5,5-tetramethyl-1,3,2-dioxaborolan-2-yl)-3-(trifluoromethyl) picolinonitrile

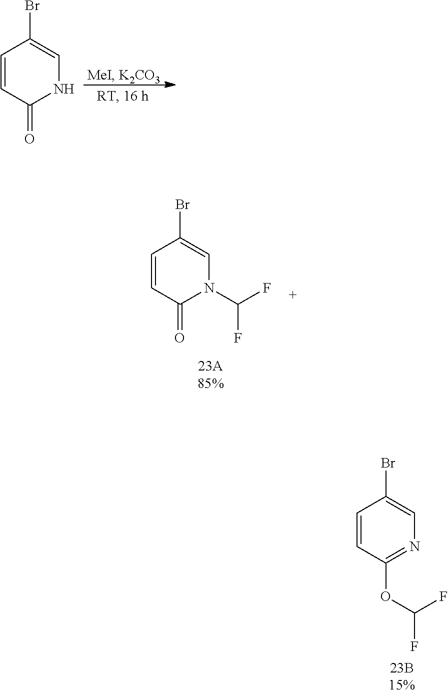 [SMILES: O=c1ccc(Br)c[nH]1 ; O=c1ccc(Br)cn1C(F)F ; FC(F)Oc1ccc(Br)cn1]

Analogous to the procedure for the preparation of Intermediate 10, reaction of 5-bromopyridin-2(1H)-one 2,2-difluoroethyl difluoro (700 mg, 1.23 mmol) and iodomethane as alkylation agent resulted in Intermediate 23A (460 mg, 69% yield) as an off-white solid; LCMS Method F: RT=1.08 min, [M+H]⁺=223.1 and Intermediate 23B (100 mg, 15% yield) as a brown color semi-solid; LCMS Method F: RT=1.65 min, [M+H]⁺=223.1.

### Intermediate 24. 1-(difluoromethyl)-5-(4,4,5,5-tetramethyl-1,3,2-dioxaborolan-2-yl)pyridin-2(1H)-one

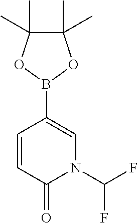 [SMILES: CC1(C)OB(c2ccc(=O)n(C(F)F)c2)OC1(C)C]

Analogous to the procedure for the preparation of Intermediate 12, 5-bromo-2-(difluoromethoxy)pyridine (300 mg, 1.35 mmol) afforded Intermediate 24 (360 mg, 89% yield) as a brown color semi-solid. MS [M+H]⁺=272.3.

### Intermediate 25. 2-(difluoromethoxy)-5-(4,4,5,5-tetramethyl-1,3,2-dioxaborolan-2-yl)pyridine

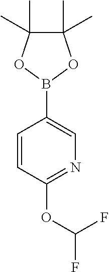 [SMILES: CC1(C)OB(c2ccc(OC(F)F)nc2)OC1(C)C]

Analogous to the procedure for the preparation of Intermediate 12, 5-bromo-2-(difluoromethoxy)pyridine (150 mg, 0.67 mmol) afforded Intermediate 25 (160 mg, 80% yield) as a brown color semi-solid. MS [M+H]⁺=272.2.

### Intermediate 26. 3-(4,4,5,5-tetramethyl-1,3,2-dioxaborolan-2-yl)-2,5-dihydro-1H-pyrrole

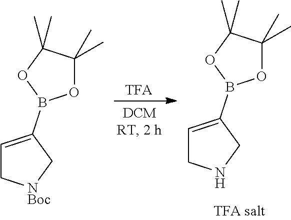 [SMILES: CC(C)(C)OC(=O)N1CC=C(B2OC(C)(C)C(C)(C)O2)C1 ; CC1(C)OB(C2=CCNC2)OC1(C)C]

TFA (13.1 mL, 169 mmol) was added dropwise to a solution of tert-butyl 3-(4,4,5,5-tetramethyl-1,3,2-dioxaborolan-2-yl)-2,5-dihydro-1H-pyrrole-1-carboxylate (2.5 g, 8.47 mmol) in DCM (50 mL) at rt. Stirred at it for 90 min and was evaporated under reduced pressure to afford Intermediate 26 (1.5 g, 7.69 mmol, 91% yield) as a brown oil. ¹H NMR (300 MHz, CDCl₃) δ ppm 6.52-6.44 (m, 1H), 6.05 (br s, 1H), 4.24 (m, 4H), 1.29 (s, 12H).

### Intermediate 27. 1-(3-(4,4,5,5-tetramethyl-1,3,2-dioxaborolan-2-yl)-2,5-dihydro-1H-pyrrol-1-yl)ethan-1-one

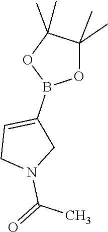 [SMILES: CC(=O)N1CC=C(B2OC(C)(C)C(C)(C)O2)C1]

Triethylamine (2.50 mL, 17.9 mmol) was added dropwise to a solution of Intermediate 26 and 3-(4,4,5,5-tetramethyl-1,3,2-dioxaborolan-2-yl)-2,5-dihydro-1H-pyrrole (700 mg, 3.59 mmol) in DCM (15 mL) at 0° C. After 5 min, acetyl chloride (0.306 mL, 4.31 mmol) was added and stirred at 0° C. for 1 h. The reaction mixture was partitioned between DCM (20 mL) and water (50 mL). Organic layer was separated, dried over sodium sulphate and concentrated under reduced pressure to afford Intermediate 27 (700 mg, 2.95 mmol, 82% yield) as a brown semisolid. MS: [M+H]⁺=238.4; ¹H NMR (400 MHz, CDCl₃) δ ppm 6.67-6.32 (m, 1H) 4.40-4.31 (m, 4H), 2.25-2.06 (s, 3H), 1.36-1.26 (s, 12H).

### Intermediate 28. methyl 3-(4,4,5,5-tetramethyl-1,3,2-dioxaborolan-2-yl)-2,5-dihydro-1H-pyrrole-1-carboxylate

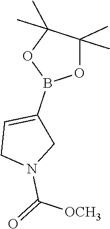 [SMILES: COC(=O)N1CC=C(B2OC(C)(C)C(C)(C)O2)C1]

Analogous to the procedure for the preparation of Intermediate 27, reaction of 3-(4,4,5,5-tetramethyl-1,3,2-dioxaborolan-2-yl)-2,5-dihydro-1H-pyrrole (700 mg, 3.59 mmol) and methyl chloroformate (0.334 mL, 4.31 mmol) afforded Intermediate 28 (900 mg, 3.56 mmol, 99% yield) as a pale brown solid. MS: [M+Na]⁺=276.3; ¹H NMR (400 MHz, CDCl₃) δ ppm 6.56-6.30 (m, 1H), 4.33-4.11 (m, 4H), 3.73 (s, 3H), 1.38-1.13 (s, 12H).

### Intermediate 29, tert-butyl (R)-(1-(4-amino-7-bromopyrrolo[2,1-f][1,2,4]triazin-5-yl)piperidin-3-yl)carbamate

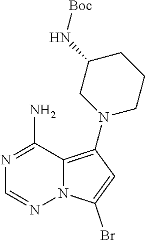 [SMILES: CC(C)(C)OC(=O)N[C@@H]1CCCN(c2cc(Br)n3ncnc(N)c23)C1]

7-Bromo-5-iodopyrrolo[2,1-f][1,2,4]triazin-4-amine (3.0 g, 8.85 mmol), tert-butyl (R)-piperidin-3-ylcarbamate (7.09 g, 35.4 mmol), Nickel(II) chloride hexahydrate (0.421 g, 1.77 mmol), tris(2,2′-bipyridine)ruthenium(II) hexafluorophosphate (0.152 g, 0.177 mmol) and DABCO (3.18 g, 28.3 mmol) were taken in a pressure tube, degassed with nitrogen and then DMSO (90 mL) was added. The reaction mixture was stirred under blue LED irradiation at it for 24 h. The mixture was diluted with THF and ethyl acetate, and washed with brine. The organic layer was concentrated, and the crude product was recrystallized from acetone-water mixture to afford Intermediate 29 (2.5 g, 69% yield) as an off-white solid. MS: [M+H]⁺=413.2; ¹H NMR (400 MHz, DMSO-d₆) δ ppm 8.00 (br s, 1H), 7.82 (s, 1H), 7.04 (br s, 1H), 6.88 (br s, 1H), 6.75 (s, 1H), 3.80-3.47 (m, 3H), 3.15-2.63 (m, 3H), 1.90-1.56 (m, 3H), 1.38 (br s, 9H).

### Intermediate 30. Methyl (R)-3-(4-amino-5-(3-((tert-butoxycarbonyl)amino)piperidin-1-yl)pyrrolo[2,1-f][1,2,4]triazin-7-yl)-2,5-dihydro-1H-pyrrole-1-carboxylate

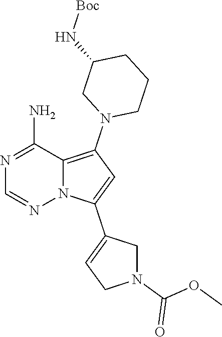 [SMILES: COC(=O)N1CC=C(c2cc(N3CCC[C@@H](NC(=O)OC(C)(C)C)C3)c3c(N)ncnn23)C1]

To a solution of Intermediate 29 (1.0 g, 2.431 mmol) and Intermediate 28 (0.738 g, 2.92 mmol) in THF (10 mL), was added aqueous potassium phosphate tribasic anhydrous (3.04 mL 2M, 6.08 mmol). The mixture was purged with argon for 3 min, charged with PdCl₂(dppf)-CH₂Cl₂ adduct (0.119 g, 0.146 mmol) and heated at 80° C. for 2 h. After cooling, reaction mixture was diluted with ethyl acetate and brine solution. Organic layer was separated, dried over Na₂SO₄, filtered and concentrated to afford brown gummy solid, which was purified by silica gel chromatography (0-8% MeOH in DCM) which afforded Intermediate 30 (820 mg, 74% yield) as a yellow solid. MS: [M+H]⁺=458.3; ¹H NMR (400 MHz, DMSO-d₆) δ ppm 7.85-7.82 (m, 2H), 7.02 (br s, 1H), 6.90 (br s, 1H), 6.81-6.67 (m, 2H), 4.53 (br d, J=10.5 Hz, 2H), 4.29 (br s, 2H), 3.66 (d, J=4.5 Hz, 3H), 3.37-332 (m, 2H), 3.03 (br s, 1H), 2.85 (br s, 1H), 2.68 (br s, 1H), 1.75 (br s, 2H), 1.66 (br s, 1H), 1.38 (s, 9H), 1.30-1.14 (m, 1H).

### Intermediate 31. Methyl (R)-3-(4-amino-5-(3-aminopiperidin-1-yl)pyrrolo[2,1-f][1,2,4]triazin-7-yl)-2,5-dihydro-1H-pyrrole-1-carboxylate, HCl salt

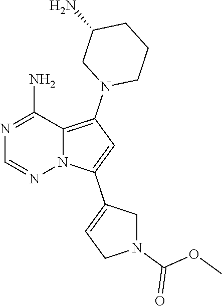 [SMILES: COC(=O)N1CC=C(c2cc(N3CCC[C@@H](N)C3)c3c(N)ncnn23)C1]

To a solution of Intermediate 30 (1.05 g, 2.30 mmol) in dioxane (10 mL), was added 4N HCl in dioxane (2.87 mL, 11.5 mmol) and stirred at rt for 4 h. Reaction was concentrated and residue was washed with diethyl ether to afford Intermediate 31 (850 mg, 86% yield) as a yellow solid. MS: [M+H]⁺=358.2; ¹H NMR (400 MHz, DMSO-d₆) δ ppm 8.12 (s, 1H), 6.96 (d, J=6.0 Hz, 1H), 6.82 (br s, 1H), 4.64-4.52 (m, 2H), 4.38-4.22 (m, 2H), 3.66 (br d, J=4.0 Hz, 5H), 3.24-3.14 (m, 1H), 3.08-2.93 (m, 2H), 2.75-2.64 (m, 1H), 2.36-2.17 (m, 1H), 2.11 (br s, 1H), 1.83 (br s, 3H).

Analogous to the synthetic procedure of Intermediate 31, the following intermediates were synthesized using the corresponding boronate, followed by Boc-deprotection using 4N HCl in dioxane to obtain the HCl salt, or using TFA to obtain the TFA salt.

| | | | MS |
|---|---|---|---|
| Intermediate | Structure | Name | (M + H)⁺ |
| 32 | 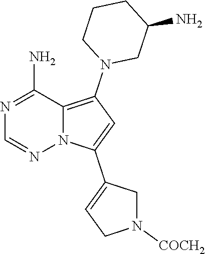 | (R)-1-(3-(4-amino-5-(3-aminopiperidin- 1-yl)pyrrolo[2,1-f][1,2,4]triazin-7-yl)- 2,5-dihydro-1H-pyrrol-1-yl)ethan-1-one | 342.2 |
| 33 | 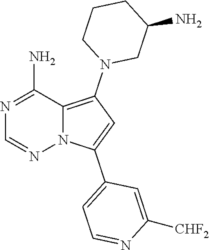 [SMILES: Nc1ncnn2c(-c3ccnc(C(F)F)c3)cc(N3CCC[C@@H](N)C3)c12] | (R)-5-(3-aminopiperidin-1-yl)-7-(2- (difluoromethyl)pyridin-4-yl)pyrrolo [2,1-f][1,2,4]triazin-4-amine | 360.4 |
| 34 | 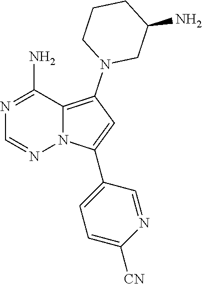 [SMILES: N#Cc1ccc(-c2cc(N3CCC[C@@H](N)C3)c3c(N)ncnn23)cn1] | (R)-5-(4-amino-5-(3-aminopiperidin- 1-yl)pyrrolo[2,1-f][1,2,4]triazin-7- yl)picolinonitrile | 335.4 |
| 34A | 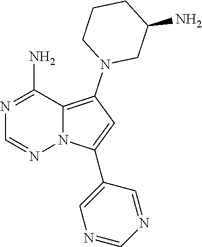 [SMILES: Nc1ncnn2c(-c3cncnc3)cc(N3CCC[C@@H](N)C3)c12] | (R)-5-(3-aminopiperidin-1-yl)- 7-(pyrimidin-5-yl)pyrrolo[2,1-f] [1,2,4]triazin-4-amine, HCl | 311.0 |

### Intermediate 35. 6-(2-morpholinoethoxy)picolinic acid TFA salt

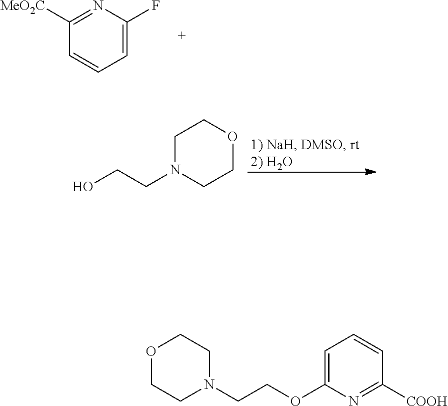 [SMILES: COC(=O)c1cccc(F)n1 ; OCCN1CCOCC1 ; O=C(O)c1cccc(OCCN2CCOCC2)n1]

To a solution of methyl 6-fluoropicolinate (400 mg, 2.58 mmol) in DMSO (4 mL) was added 2-morpholinoethan-1-ol (406 mg, 3.09 mmol) followed by the addition of NaH (206 mg, 5.16 mmol) in portions carefully at rt. The reaction was stirred under N2 at rt for 3 h. H₂O (1 mL) was carefully added. After stirring at rt for another 1 h, the reaction was acidified with TFA, and was diluted with MeOH. The crude product was purified by reverse phase chromatography to provide Intermediate 35 (623 mg, 50%). MS (ESI) m/z 253 (M+H)⁺.

By following the same procedure as described in Intermediate 35. Intermediates 36-46 were prepared.

### Intermediate 36. 6-(2-(4-methylpiperazin-1-yl)ethoxy)picolinic acid TFA salt

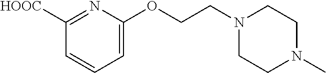

MS (ESI) m/z 266 (M+H)⁺.

### Intermediate 37. 6-(2-(dimethylamino)ethoxy)picolinic acid TFA salt

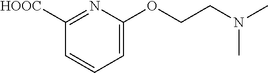

MS (ESI) m/z 211 (M+H)⁺.

### Intermediate 38. 2-(2-morpholinoethoxy)isonicotinic acid TFA salt

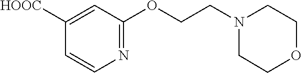 [SMILES: O=C(O)c1ccnc(OCCN2CCOCC2)c1]

MS (EST) m/z 253 (M+H)⁺.

### Intermediate 39. 2-(2-(4-methylpiperazin-1-yl)ethoxy)isonicotinic acid, TFA salt

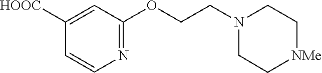 [SMILES: CN1CCN(CCOc2cc(C(=O)O)ccn2)CC1]

MS (ESI) m/z 266 (M+H)⁺.

### Intermediate 40. 2-(2-(dimethylamino)ethoxy)isonicotinic acid TFA salt

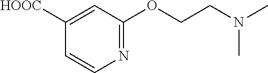

MS (ESI) m/z 211 (M+H)⁺.

### Intermediate 41. 6-((1-methylpiperidin-4-yl)oxy)picolinic acid, TFA salt

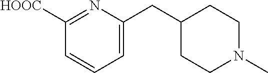 [SMILES: CN1CCC(Cc2cccc(C(=O)O)n2)CC1]

MS (ESI) m/z 237 (M+H)⁺.

### Intermediate 42. 6-((1-methylazetidin-3-yl)oxy)picolinic acid TFA salt

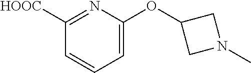

MS (ESI) m/z 209 (M+H)⁺.

### Intermediate 43. 6-((1-methylazetidin-3-yl)methoxy)picolinic acid TFA salt

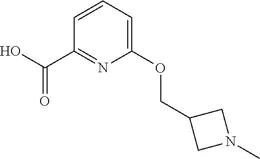 [SMILES: CN1CC(COc2cccc(C(=O)O)n2)C1]

Intermediate 43 was prepared by following the same procedure as described in Intermediate 35. MS (ESI) m/z 223 (M+H)⁺.

### Intermediate 44. 6-((1-isopropylpiperidin-4-yl)oxy)picolinic acid TFA salt

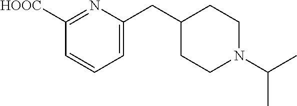 [SMILES: CC(C)N1CCC(Cc2cccc(C(=O)O)n2)CC1]

MS (ESI) m/z 265 (M+H)⁺.

### Intermediate 45. 2-(2-(dimethylamino)ethoxy)nicotinic acid TFA salt

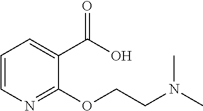

MS (ESI) m/z 211 (M+H)⁺.

### Intermediate 46. 6-(quinuclidin-3-yloxy)picolinic acid, TFA salt

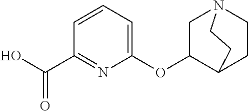 [SMILES: O=C(O)c1cccc(OC2CN3CCC2CC3)n1]

MS (ESI) m/z 249 (M+H)⁺.

### Intermediate 47A. methyl 3-(2-(dimethylamino)ethoxy)isonicotinate TFA salt

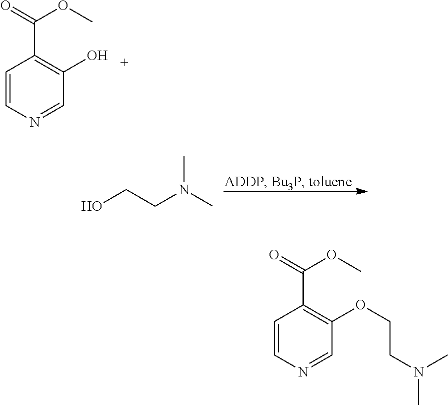

To a solution of methyl 3-hydroxyisonicotinate (100 mg, 0.653 mmol) in toluene (4 mL) was added 2-(dimethylamino)ethan-1-ol (0.131 mL, 1.31 mmol) followed by the addition of 1,1′-(azodicarbonyl)dipiperidine (494 mg, 1.96 mmol) and tributylphosphine (0.489 mL, 1.96 mmol) at rt. The reaction was heated with microwave at 150° C. for 30 min. The solvent was removed. The crude product was purified by reverse phase chromatography to give Intermediate 47A (202 mg, 68%). MS (ESI) m/z 225 (M+H)⁺; ¹H NMR (500 MHz, CD₃OD) δ 8.65 (s, 1H), 8.46 (d, J=5.0 Hz, 1H), 7.85 (d, J=5.0 Hz, 1H), 4.69-4.60 (m, 2H), 3.97 (s, 3H), 3.73-3.67 (m, 2H), 3.09 (s, 6H).

### Intermediate 47. 3-(2-(dimethylamino)ethoxy)isonicotinic acid

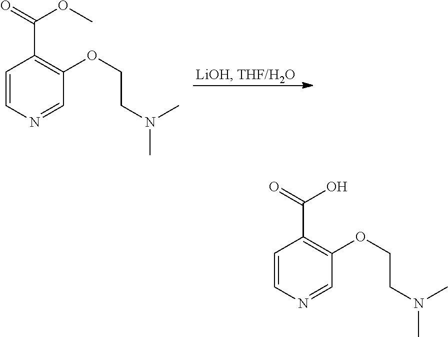 [SMILES: COC(=O)c1ccncc1OCCN(C)C ; CN(C)CCOc1cnccc1C(=O)O]

To a solution of Intermediate 47A (202 mg, 0.447 mmol) in THF (3 mL) and H₂O (1 mL) was added LiOH (42.8 mg, 1.786 mmol) at rt. The reaction was stirred under N2 at rt for 3 h. The reaction was neutralized with 1.0 N HCl solution. The solvent was removed and the crude product was dried to give a white solid, which was used without further purification. MS (ESI) m/z 211 (M+H)⁺.

### Intermediate 48A. methyl 6-morpholinopicolinate

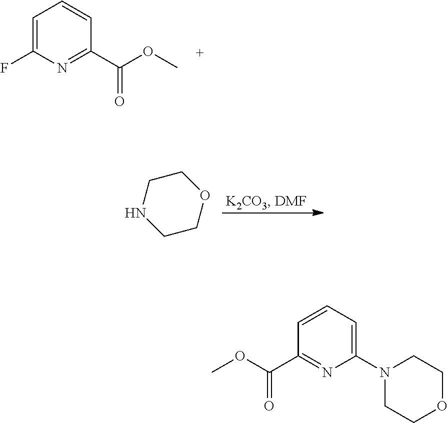 [SMILES: COC(=O)c1cccc(F)n1 ; C1COCCN1 ; COC(=O)c1cccc(N2CCOCC2)n1]

To a solution of methyl 6-fluoropicolinate (300 mg, 1.93 mmol) in DMF (3 mL) was added morpholine (202 mg, 2.32 mmol) followed by the addition of K₂CO₃ (535 mg, 3.87 mmol) at rt. The reaction was heated with microwave at 150° C. for 15 min. The reaction mixture was diluted with EtOAc, washed with H₂O and brine. The organic phase was dried over Na₂SO₄, filtered and concentrated. The crude product was purified by normal phase chromatography to give Intermediate 48A (340 mg, 79%) was obtained. MS (ESI) m/z 223 (M+H); ¹H NMR (500 MHz, CDCl₃) δ 7.61 (dd, J=8.6, 7.4 Hz, 1H), 7.47 (d, J=7.2 Hz, 1H), 6.81 (d, J=8.2 Hz, 1H), 3.95 (s, 3H), 3.87-3.80 (m, 4H), 3.65-3.56 (m, 4H).

### Intermediate 48. 6-morpholinopicolinic acid

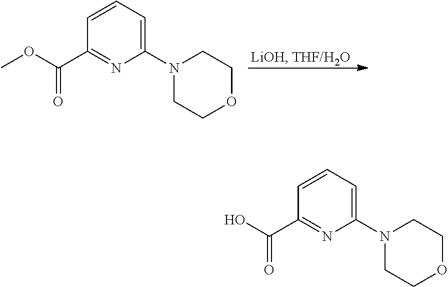 [SMILES: COC(=O)c1cccc(N2CCOCC2)n1 ; O=C(O)c1cccc(N2CCOCC2)n1]

To a solution of Intermediate 48A (340 mg, 1.530 mmol) in THF (4 mL) was added H₂O (2 mL) followed by the addition of LiOH (73 mg, 3.06 mmol) at rt. The reaction was stirred under N2 at rt for 3 h. The reaction was neutralized with 1.0 N HCl solution (~3 mL). The solvent was removed to give a white solid, which was used without further workup. MS (ESI) m/z 209 (M+H)⁺.

### Intermediate 49. 6-(4-methylpiperazin-1-yl)picolinic acid

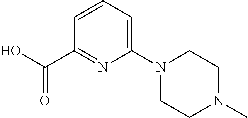 [SMILES: CN1CCN(c2cccc(C(=O)O)n2)CC1]

Intermediate 49 was prepared by following the same procedure as described in Intermediate 48. MS (ESI) m/z 222 (M+H)⁺.

### Intermediate 50. 4-methyl-6-(4-methylpiperazin-1-yl)picolinic acid

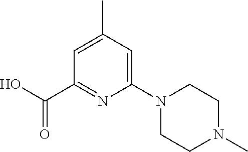 [SMILES: Cc1cc(C(=O)O)nc(N2CCN(C)CC2)c1]

Intermediate 50 was prepared by following the same procedure as described in Intermediate 48 using ethyl 6-chloro-4-methylpyridine-2-carboxylate. MS (ESI) m/z 236 (M+H)⁺.

Intermediate 51A. methyl 3-fluoro-6-(4-methylpiperazin-1-yl)picolinate TFA salt, and Intermediate 51B. methyl 6-fluoro-3-(4-methylpiperazin-1-yl)picolinate TFA salt

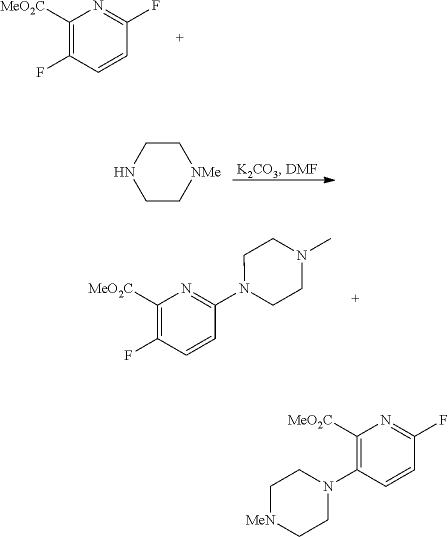 [SMILES: COC(=O)c1nc(F)ccc1F ; CN1CCNCC1 ; COC(=O)c1nc(N2CCN(C)CC2)ccc1F ; COC(=O)c1nc(F)ccc1N1CCN(C)CC1]

To a solution of methyl 3,6-difluoropicolinate (700 mg, 4.04 mmol) in DMF (9 mL) was added 1-methylpiperazine (446 mg, 4.45 mmol) followed by the addition of K₂CO₃ (838 mg, 6.07 mmol) at rt. The reaction was stirred under N2 at 80° C. for 5 h. The reaction mixture was diluted with EtOAc, washed with H₂O and brine. The organic phase was dried over Na₂SO₄, filtered and concentrated. This product was purified by reverse phase prep HPLC to give Intermediate 51A (354 mg, 35%) and Intermediate 51B (610 mg, 60%). Intermediate 51A: MS (ESI) m/z 254 (M+H)⁺; ¹H NMR (500 MHz, CD₃OD) δ 7.58 (t, J=9.4 Hz, 1H), 7.19 (dd, J=9.3, 2.6 Hz, 1H), 4.50 (br d, J=12.8 Hz, 2H), 3.95 (s, 3H), 3.60 (br d, J=10.5 Hz, 2H), 3.31-3.10 (m, 4H), 2.96 (s, 3H). Intermediate 51B: MS (ESI) m/z 254 (M+H)⁺; ¹H NMR (500 MHz, CDCl₃) δ 7.74 (dd, J=8.7, 6.5 Hz, 1H), 7.16 (dd, J=8.9, 3.9 Hz, 1H), 3.97 (s, 3H), 3.73 (br d, J=12.2 Hz, 2H), 3.45-3.36 (m, 4H), 3.20 (br s, 2H), 2.96 (s, 3H)

### Intermediate 51. 3-fluoro-6-(4-methylpiperazin-1-yl)picolinic acid

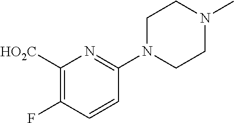 [SMILES: CN1CCN(c2ccc(F)c(C(=O)O)n2)CC1]

Intermediate 51 was prepared by following a similar procedure as described in Intermediate 48 from Intermediate 51A. MS (ESI) m/z 240 (M+H)⁺.

### Intermediate 52. 6-fluoro-3-(4-methylpiperazin-1-yl)picolinic acid

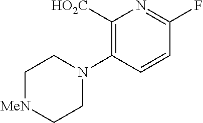 [SMILES: CN1CCN(c2ccc(F)nc2C(=O)O)CC1]

Intermediate 52 was prepared by following a similar procedure as described in Intermediate 48 from Intermediate 51B. MS (ESI) m/z 240 (M+H)⁺.

### Intermediate 53A. tert-butyl 4-(6-(methoxycarbonyl)pyridin-2-yl)piperazine-1-carboxylate

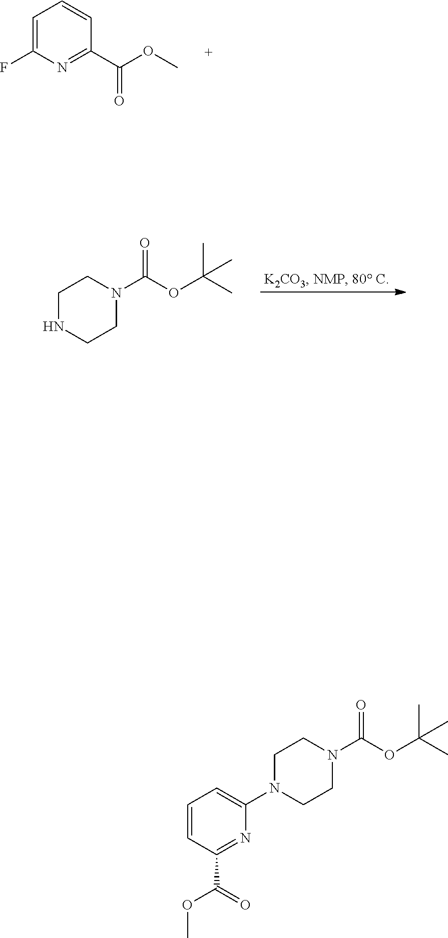

To a solution oft-butyl piperazine-1-carboxylate (3.60 g, 19.34 mmol) in NMP (10 mL) was added K₂CO₃ (3.56 g, 25.8 mmol) followed by addition of methyl 6-fluoropicolinate (2.0 g, 12.89 mmol) at rt. The reaction was heated at 80° C. for 16 h. The reaction mixture was diluted with EtOAc, washed with H₂O and brine. The organic phase was dried over Na₂SO₄, filtered and concentrated. The crude product was purified by normal phase chromatography to give Intermediate 53A (3.6 g, 87%). MS (ESI) m/z 322 (M+H)⁺; ¹H NMR (500 MHz, CDCl₃) δ 7.59 (dd, J=8.5, 7.4 Hz, 1H), 7.44 (d, J=7.4 Hz, 1H), 6.81 (d, J=8.6 Hz, 1H), 3.93 (s, 3H), 3.64-3.58 (m, 4H), 3.57-3.52 (m, 4H), 1.48 (s, 9H).

### Intermediate 53B. methyl 6-(piperazin-1-yl)picolinate

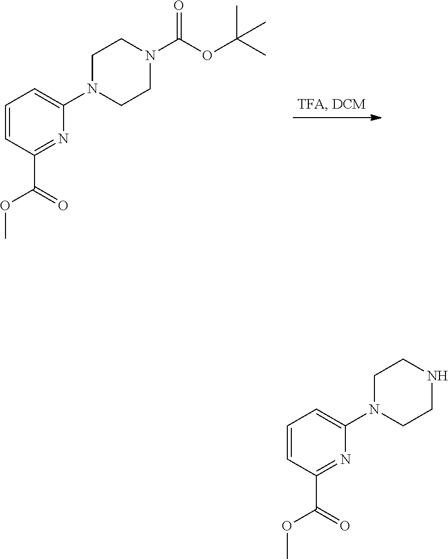 [SMILES: COC(=O)c1cccc(N2CCN(C(=O)OC(C)(C)C)CC2)n1 ; COC(=O)c1cccc(N2CCNCC2)n1]

To a solution of Intermediate 53A (3.60 g, 11.2 mmol) in DCM (15 mL) was added TFA (5 mL, 64.9 mmol) dropwise at rt. The reaction was stirred under N₂ at it for overnight. The reaction mixture was diluted with CH₂Cl₂, washed with saturated NaHCO₃. The aq. phase was back extracted with DCM. The combined organic phase was further washed with brine. The organic phase was dried over Na₂SO₄, filtered and concentrated to provide Intermediate 53B (2.25 g, 91%). MS (ESI) m/z 222 (M+H)⁺; ¹H NMR (500 MHz, CDCl₃) δ 7.57 (dd, J=8.6, 7.4 Hz, 1H), 7.41 (d, J=7.4 Hz, 1H), 6.80 (d, J=8.6 Hz, 1H), 3.93 (s, 3H), 3.58 (dd, J=5.9, 4.2 Hz, 4H), 2.98 (dd, J=6.0, 4.2 Hz, 4H).

### Intermediate 53C. methyl 6-(4-(2,2-difluoroethyl)piperazin-1-yl)picolinate

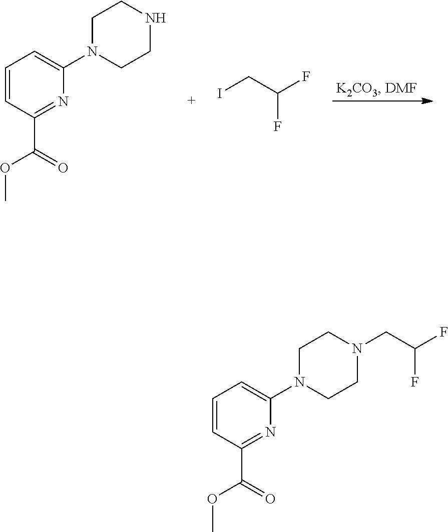 [SMILES: COC(=O)c1cccc(N2CCNCC2)n1 ; FC(F)CI ; COC(=O)c1cccc(N2CCN(CC(F)F)CC2)n1]

To a solution of Intermediate 53B (150 mg, 0.678 mmol) in DMF (2 mL) was added 1,1-difluoro-2-iodoethane (195 mg, 1.017 mmol) followed by addition of K₂CO₃ (234 mg, 1.695 mmol) at rt. The reaction was stirred under N₂ at 80° C. for 16 h. The reaction mixture was diluted with EtOAc, washed with H₂O and brine. The organic phase was dried over Na₂SO₄, filtered and concentrated. The crude product was purified by normal phase chromatography to provide Intermediate 53C (185 mg, 96%). MS (ESI) m/z 286 (M+H)⁺; ¹H NMR (500 MHz, CDCl₃) δ 7.60 (dd, J=8.6, 7.4 Hz, 1H), 7.44 (d, J=7.4 Hz, 1H), 6.82 (d, J=8.6 Hz, 1H), 6.08-5.80 (m, 1H), 3.95 (s, 3H), 3.65 (br d, J=4.9 Hz, 4H), 2.81 (td, J=15.0, 4.3 Hz, 2H), 2.74-2.68 (m, 4H). ¹⁹F NMR (471 MHz, CDCl₃) δ −118.27 (s, 2F).

### Intermediate 53. 6-(4-(2,2-difluoroethyl)piperazin-1-yl)picolinic acid

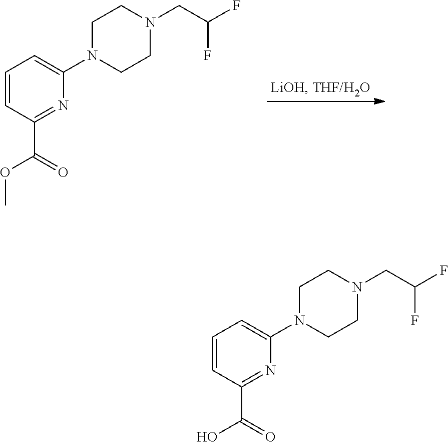 [SMILES: COC(=O)c1cccc(N2CCN(CC(F)F)CC2)n1 ; O=C(O)c1cccc(N2CCN(CC(F)F)CC2)n1]

To a solution of methyl Intermediate 53C (165 mg, 0.578 mmol) in THF (3 mL) was added LiOH (27.7 mg, 1.16 mmol) followed by the addition of H₂O (1 mL) at it. The reaction was stirred under N₂ at rt for 4 h. The reaction was neutralized with 1.0 N HCl solution. The solvent was removed to give a white solid, which was used without further purification. MS (EST) m/z 272 (M+H)⁺.

### Intermediate 54. 6-(4-tert-butoxycarbonyl)piperazin-1-yl)picolinic acid

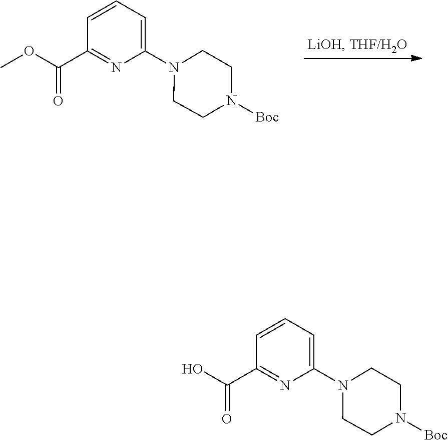 [SMILES: COC(=O)c1cccc(N2CCN(C(=O)OC(C)(C)C)CC2)n1 ; CC(C)(C)OC(=O)N1CCN(c2cccc(C(=O)O)n2)CC1]

To a solution of Intermediate 53A (137 mg, 0.426 mmol) in THF (4 mL) was added H₂O (2 mL) followed by addition of LiOH (30.6 mg, 1.28 mmol) at rt. The reaction was stirred under N₂ at rt for 2 h. The reaction was neutralized with 1.0 N HCl solution. The solvent was removed to give a white solid, which was used without further purification. MS (ESI) m/z 308 (M+H)⁺.

### Intermediate 55. 6-((1R,5S)-8-(tert-butoxycarbonyl)-3,8-diazabicyclo[3.2.1]octan-3-yl)picolinic acid

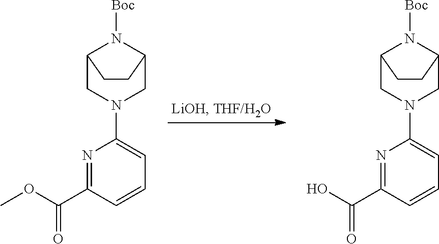 [SMILES: COC(=O)c1cccc(N2CC3CCC(C2)N3C(=O)OC(C)(C)C)n1 ; CC(C)(C)OC(=O)N1C2CCC1CN(c1cccc(C(=O)O)n1)C2]

Intermediate 55 was prepared by following a similar procedure to that described in Intermediate 54 by using tert-butyl 3,8-diazabicyclo[3.2.1]octane-8-carboxylate. MS (ESI) m/z 334 (M+H)⁺.

### Intermediate 56. 6-(4-methylpiperazin-1-yl)pyrazine-2-carboxylic acid

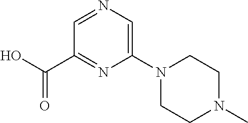 [SMILES: CN1CCN(c2cncc(C(=O)O)n2)CC1]

Intermediate 56 was prepared by following the same procedure as described in Intermediate 48 using methyl 6-chloropyrazine-2-carboxylate. MS (ESI) m/z 223 (M+H)⁺.

Intermediate 57A. methyl 6-((4-methylpiperazin-1-yl)methyl)picolinate

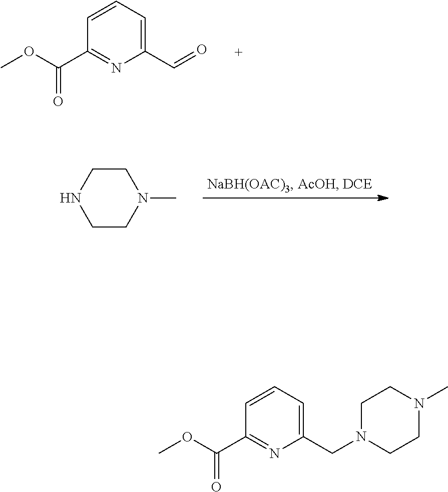

To a solution of 6-formyl-2-pyridine carboxylic acid methyl ester (220 mg, 0.33 mmol) in DCE (4 mL) was added 1-methylpiperazine (160 mg, 1.60 mmol) followed by addition of NaBH(OAc)₃ (565 mg, 2.66 mmol) and acetic acid (0.114 mL, 2.00 mmol) at it. The reaction was stirred under N₂ at rt for 5 h. The reaction mixture was diluted with EtOAc, washed with saturated NaHCO₃ and brine. The organic phase was dried over Na₂SO₄, filtered and concentrated. The crude product was purified by reverse phase chromatography to give Intermediate 57A (65 mg, 20%). MS (ESI) m/z 250 (M+H)⁺; ¹H NMR (500 MHz, CD₃OD) δ 8.20-8.12 (m, 1H), 8.08 (t, J=7.8 Hz, 1H), 7.79 (d, J=7.8 Hz, 1H), 4.36 (s, 2H), 4.02 (s, 3H), 3.55 (br s, 4H), 3.40 (br s, 4H), 2.98 (s, 3H).

### Intermediate 57. 6-((4-methylpiperazin-1-yl)methyl)picolinic acid

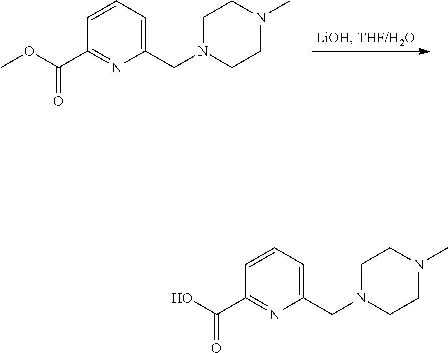

To a solution of Intermediate 57A (65 mg, 0.261 mmol) in THF (3 mL) and H₂O (1 mL) was added LiOH (31 mg, 1.304 mmol) at it. The reaction was stirred under N₂ at rt for 3 h. The reaction was neutralized with 1.0 N HCl, and the solvent was removed to give Intermediate 57 as a crude product that was used without further workup. MS (ESI) m/z 236 (M+H)⁺.

### Intermediate 58. 2-(4-methylpiperazin-1-yl)pyrimidine-4-carboxylic acid

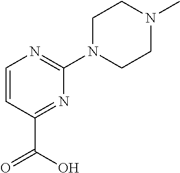 [SMILES: CN1CCN(c2nccc(C(=O)O)n2)CC1]

Intermediate 58 was prepared by following the same procedure as described in Intermediate 48 methyl 2-chloropyrimidine-4-carboxylate. MS (ESI) m/z 223 (M+H)⁺.

### Intermediate 59. 2-(6-isopropyl-2,6-diazaspiro[3.3]heptan-2-yl)pyrimidine-4-carboxylic acid

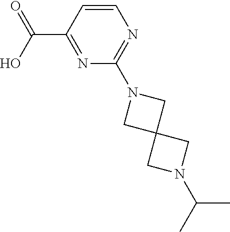 [SMILES: CC(C)N1CC2(CN(c3nccc(C(=O)O)n3)C2)C1]

Intermediate 59 was prepared by following the same procedure as described in Intermediate 48 using methyl 2-chloropyrimidine-4-carboxylate and 2-isopropyl-2,6-diazaspiro[3.3]heptane. MS (ESI) m/z 263 (M+H)⁺.

### Intermediate 60A. methyl 5-chloro-3-(((1R,3s,5S)-8-methyl-8-azabicyclo[3.2.1]octan-3-yl)oxy)thiophene-2-carboxylate

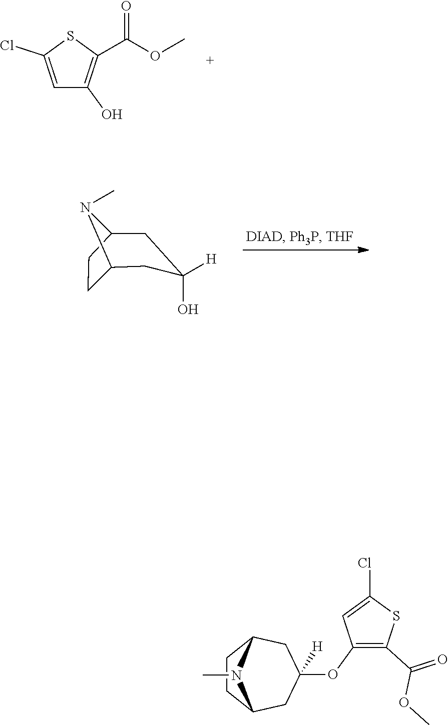

To a solution of Methyl 5-chloro-3-hydroxythiophene-2-carboxylate (1.0 g, 5.19 mmol) in THF (20 mL) was added tropine (0.806 g, 5.71 mmol) followed by addition of triphenylphosphine (2.043 g, 7.79 mmol) and DIAD (1.514 mL, 7.79 mmol) at rt. The reaction was stirred at rt for 2 h. The crude product was purified by normal phase chromatography to give Intermediate 60A

(1.22 g, 74.4%). MS (ESI) m/z 316 (M+H)⁺. ¹H NMR (500 MHz, CDCl₃) δ 6.70 (s, 1H), 4.43 (tt, J=9.7, 7.0 Hz, 1H), 3.80 (s, 3H), 3.30-3.23 (m, 2H), 2.38 (s, 3H), 2.11-2.03 (m, 2H), 1.98-1.90 (m, 4H), 1.62-1.54 (m, 2H)

### Intermediate 60. 5-chloro-3-(((1R,3s,5S)-8-methyl-8-azabicyclo[3.2.1]octan-3-yl)oxy)thiophene-2-carboxylic acid

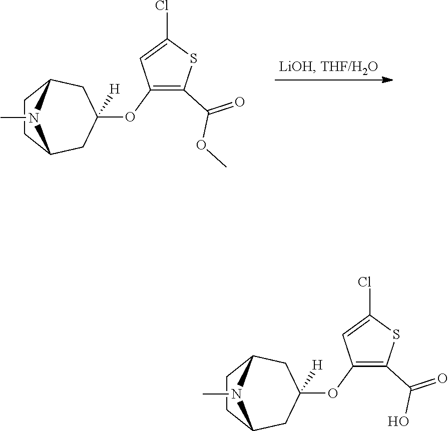 [SMILES: COC(=O)c1sc(Cl)cc1O[C@@H]1C[C@H]2CC[C@@H](C1)N2C ; CN1[C@@H]2CC[C@H]1C[C@H](Oc1cc(Cl)sc1C(=O)O)C2]

To a solution of Intermediate 60A (900 mg, 2.85 mmol) in THF (10 mL) and H₂O (2 mL) was added LiOH (102 mg, 4.27 mmol) at rt. The reaction was stirred under N₂ at rt for 16 h. It was neutralized with 1.0 N HCl, and the solvent was removed. MS (ESI) m/z 302 (M+H)⁺.

### Intermediate 61. (R)-5-(3-aminopiperidin-1-yl)-7-bromopyrrolo[2,1-f][1,2,4]triazin-4-amine TFA salt

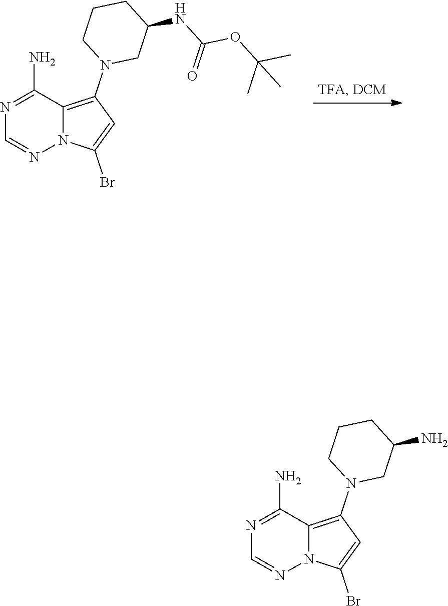 [SMILES: CC(C)(C)OC(=O)N[C@@H]1CCCN(c2cc(Br)n3ncnc(N)c23)C1 ; Nc1ncnn2c(Br)cc(N3CCC[C@@H](N)C3)c12]

To a solution of Intermediate 29 (500 mg, 1.216 mmol) in DCM (8 mL) was added TFA (2 mL) dropwise at rt. Tc reaction was stirred under N₂ at it for 1 h. The solvent was removed to provide Intermediate 61 (660 mg, 100%) as a brown solid. MS (ESI) m/z 311/313 (M+H)⁺.

### Intermediate 62. (R)—N-(1-(4-amino-7-bromopyrrolo[2,1-f][1,2,4]triazin-5-yl)piperidin-3-yl)-6-(4-methylpiperazin-1-yl)picolinamide

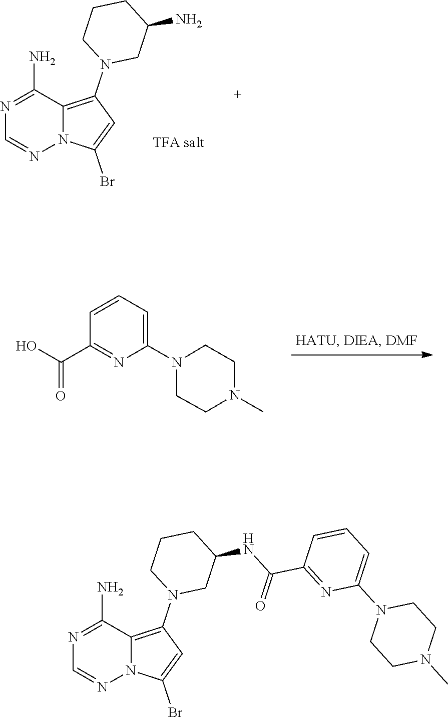 [SMILES: Nc1ncnn2c(Br)cc(N3CCC[C@@H](N)C3)c12 ; CN1CCN(c2cccc(C(=O)O)n2)CC1 ; CN1CCN(c2cccc(C(=O)N[C@@H]3CCCN(c4cc(Br)n5ncnc(N)c45)C3)n2)CC1]

To a solution of Intermediate 61 (487 mg, 0.904 mmol) in DMF (10 mL) was added Intermediate 49 (200 mg, 0.904 mmol) followed by addition of HATU (412 mg, 1.085 mmol) and DIEA (0.789 mL, 4.52 mmol) at 0° C. The reaction was stirred under N₂ at 0° C. for 1 h. The reaction mixture was diluted with EtOAc, washed with H₂O and brine. The organic phase was dried over Na₂SO₄, filtered and concentrated to give Intermediate 62, which was used as crude (480 mg, 100%). MS (ESI) m/z 514 (M+H)⁺.

### Intermediate 63A. tert-butyl 6-(6-(methoxycarbonyl)pyridin-2-yl)-2,6-diazaspiro[3.3]heptane-2-carboxylate

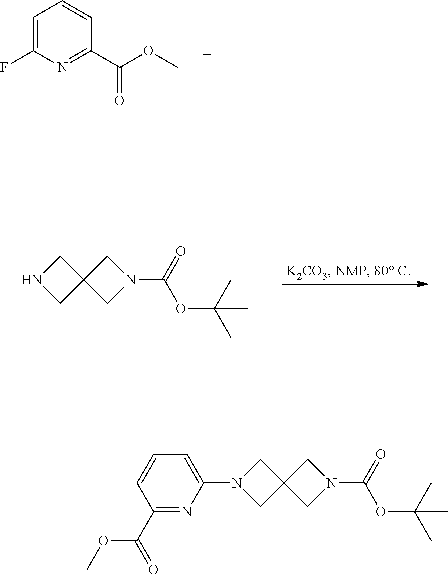 [SMILES: COC(=O)c1cccc(F)n1 ; CC(C)(C)OC(=O)N1CC2(CNC2)C1 ; COC(=O)c1cccc(N2CC3(CN(C(=O)OC(C)(C)C)C3)C2)n1]

To a solution of 2-Boc-2,6-diaza-spiro[3.3]heptane (773 mg, 3.90 mmol) in DMF (10 mL) was added K₂CO₃ (980 mg, 7.09 mmol) followed by addition of Methyl 6-fluoropicolinate (550 mg, 3.55 mmol) at rt. The reaction was stirred under N₂ at 80° C. for 16 h. The reaction mixture was diluted with EtOAc, washed with H₂O and brine. The organic phase was dried over Na₂SO₄, filtered and concentrated. Purification by normal phase chromatography provide Intermediate 63A (620 mg, 53%) as a white solid. MS (ESI) m/z 334 (M+H)⁺. ¹H NMR (500 MHz, CDCl₃) δ 7.56 (dd, J=8.2, 7.4 Hz, 1H), 7.45 (dd, J=7.4, 0.7 Hz, 1H), 6.47 (dd, J=8.3, 0.8 Hz, 1H), 4.19 (s, 4H), 4.11 (s, 4H), 3.94 (s, 3H), 1.48-1.43 (m, 9H)

### Intermediate 63B. methyl 6-(2,6-diazaspiro[3.3]heptan-2-yl)picolinate, TFA salt

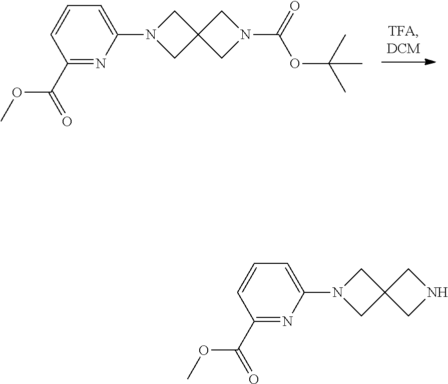

To a solution of Intermediate 63A (620 mg, 1.860 mmol) in DCM (4 mL) was added TFA (1 mL) at rt. The reaction was stirred under N₂ at rt for 1 h. The solvent was removed. MS (ESI) m/z 234 (M+H)⁺.

### Intermediate 63C. methyl 6-(6-isopropyl-2,6-diazaspiro[3.3]heptan-2-yl)picolinate

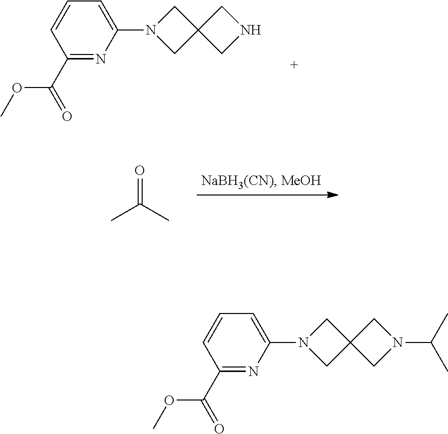

To a solution of Intermediate 63B (640 mg, 1.843 mmol) in MeOH (10 mL) was added acetone (214 mg, 3.69 mmol) followed by addition of sodium cyanoborohydride (139 mg, 2.211 mmol) at 0° C. The reaction was stirred under N₂ from 0° C. to it for 6 h. The solvent was removed. The reaction mass was diluted with EtOAc, washed with 1 M K₂HPO₄ and brine. The organic phase was dried over Na₂SO₄, filtered and concentrated. The crude product was purified by normal phase chromatography to give Intermediate 63C (505 mg, 100%). MS (ESI) m/z 276 (M+H)⁺. ¹H NMR (500 MHz, CDCl₃) δ 7.59 (dd, J=8.3, 7.4 Hz, 1H), 7.52-7.46 (m, 1H), 6.53 (dd, J=8.3, 0.7 Hz, 1H), 4.28 (br s, 4H), 3.94 (s, 3H), 4.91-3.40 (br m, 4H), 3.21-3.10 (m, 1H), 1.30 (d, J=6.4 Hz, 6H).

### Intermediate 63. 6-(6-isopropyl-2,6-diazaspiro[3.3]heptan-2-yl)picolinic acid

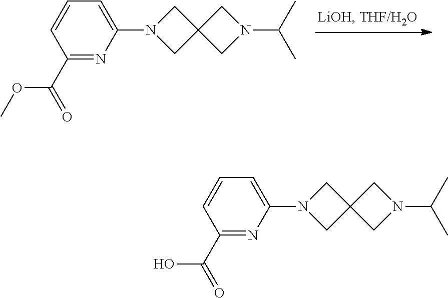 [SMILES: COC(=O)c1cccc(N2CC3(C2)CN(C(C)C)C3)n1 ; CC(C)N1CC2(CN(c3cccc(C(=O)O)n3)C2)C1]

To a solution of Intermediate 63C (505 mg, 1.834 mmol) in THF (8 mL) and H₂O (2 mL) was added LiOH (88 mg, 3.67 mmol) at rt. The reaction was stirred under N₂ at it for 4 h. The reaction was neutralized with 1.0 N HCl solution. The solvent was removed to leave a white solid of Intermediate 63 which was used without further purification. MS (ESI) m/z 262 (M+H)⁺.

### Intermediate 64. (R)—N-(1-(4-amino-7-bromopyrrolo[2,1-f][1,2,4]triazin-5-yl)piperidin-3-yl)-6-(6-isopropyl-2,6-diazaspiro[3.3]heptan-2-yl)picolinamide TFA salt

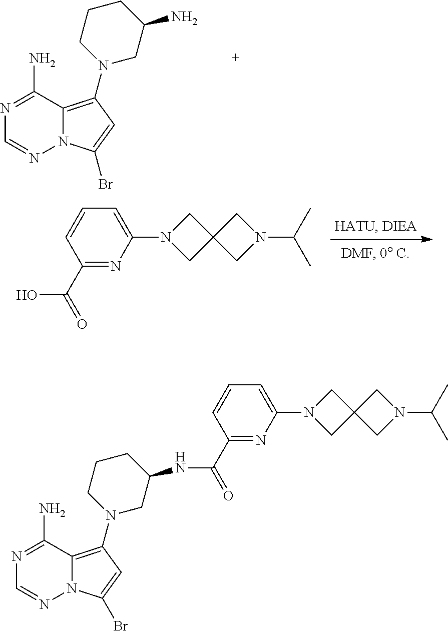

To a solution of Intermediate 61 (100 mg, 0.321 mmol) in DMF (4 mL) was added Intermediate 63 (92 mg, 0.353 mmol) followed by addition of DIEA (0.168 mL, 0.964 mmol) and HATU (147 mg, 0.386 mmol) at 0° C. The reaction was stirred under N₂ at 0° C. for 1 h. The reaction was quenched by adding H₂O. The crude product was purified by reverse phase chromatography to afford Intermediate 64 (150 mg, 60%). MS (ESI) m/z 554/556 (M+H)⁺.

¹H NMR (500 MHz, METHANOL-d₄) δ 7.96 (s, 1H), 7.73 (dd, J=8.4, 7.3 Hz, 1H), 7.41 (d, J=7.4 Hz, 1H), 6.94 (s, 1H), 6.68 (dd, J=8.5, 0.7 Hz, 1H), 4.47-4.41 (m, 2H), 4.40-4.34 (m, 4H), 4.34-4.27 (m, 1H), 4.26 (s, 2H), 3.52-3.42 (m, 1H), 3.38-3.34 (m, 1H), 3.22-3.14 (m, 1H), 3.05-2.88 (m, 2H), 2.11-2.03 (m, 1H), 2.01-1.93 (m, 2H), 1.78-1.66 (m, 1H), 1.27 (d, J=6.4 Hz, 6H).

### Intermediate 65A. methyl 5-cyano-3-(((1R,3s,5S)-8-methyl-8-azabicyclo[3.2.1]octan-3-yl)oxy)thiophene-2-carboxylate

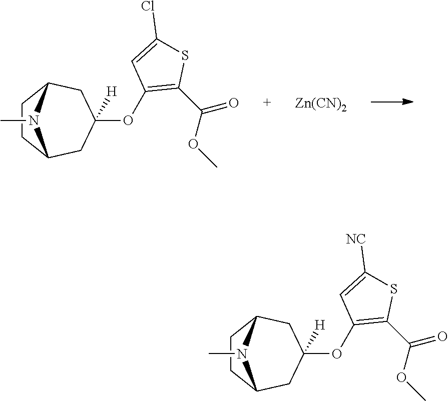

A pressure vial charged with Intermediate 60A (60 mg, 0.190 mmol), ZINC CYANIDE (446 mg, 3.80 mmol), zinc (248 mg, 3.80 mmol) and RuPhos-Pd-G3 (47.7 mg, 0.057 mmol) was degassed and then DMF (2 mL) was added. It was bubbled with N₂ for 5 min at rt. The reaction was heated at 90° C. for 2 h. The reaction was filtered. The crude product was purified by reverse phase chromatography to give Intermediate 65A (55 mg, 94%). MS (ESI) m/z 307 (M+H)⁺.

### Intermediate 65. 5-cyano-3-(((1R,3s,5S)-8-methyl-8-azabicyclo[3.2.1]octan-3-yl)oxy)thiophene-2-carboxylic acid

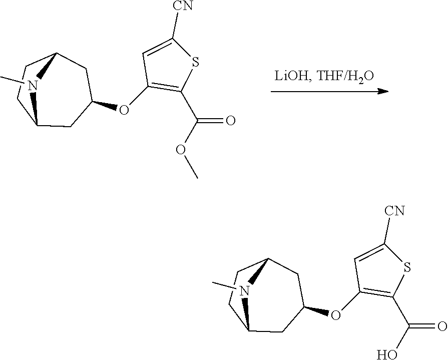 [SMILES: COC(=O)c1sc(C#N)cc1O[C@@H]1C[C@H]2CC[C@@H](C1)N2C ; CN1[C@@H]2CC[C@H]1C[C@H](Oc1cc(C#N)sc1C(=O)O)C2]

To a solution of Intermediate 65A (55 mg, 0.180 mmol) in THF (2 mL) and H₂O (1 mL) was added LiOH (12.90 mg, 0.539 mmol) at rt. The reaction was stirred under N₂ at rt for 2 h. The reaction was neutralized with 1.0 N HCl solution. Most solvent was removed. The crude product was purified by reverse phase chromatography to provide Intermediate 65 (16 mg, 31%).

MS (ESI) m/z 293 (M+H)⁺.

### Intermediate 66. 5-methyl-3-(((1R,3s,5S)-8-methyl-8-azabicyclo[3.2.1]octan-3-yl)oxy)thiophene-2-carboxylic acid

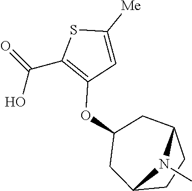 [SMILES: Cc1cc(O[C@@H]2C[C@H]3CC[C@@H](C2)N3C)c(C(=O)O)s1]

Intermediate 66 was prepared by following a similar procedure as described in Intermediate 60. MS (EST) m/z 282 (M+H)⁺.

### Example 1

### Preparation of ethyl (S)-1-(4-amino-7-(4-(methylsulfonyl)phenyl)pyrrolo[2,1-f][1,2,4]triazin-5-yl)piperidine-3-carboxylate

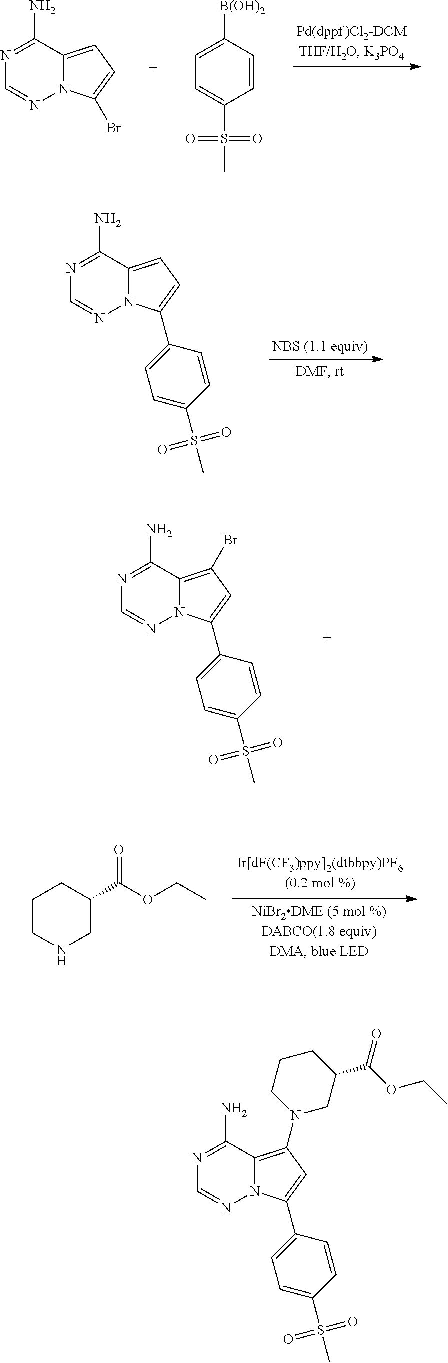 [SMILES: Nc1ncnn2c(Br)ccc12 ; CS(=O)(=O)c1ccc(B(O)O)cc1 ; CS(=O)(=O)c1ccc(-c2ccc3c(N)ncnn23)cc1 ; CS(=O)(=O)c1ccc(-c2cc(Br)c3c(N)ncnn23)cc1 ; CCOC(=O)[C@H]1CCCNC1 ; CCOC(=O)[C@H]1CCCN(c2cc(-c3ccc(S(C)(=O)=O)cc3)n3ncnc(N)c23)C1]

### Example 1A. 7-(4-(Methylsulfonyl)phenyl)pyrrolo[2,1-f][1,2,4]triazin-4-amine

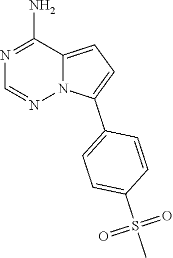 [SMILES: CS(=O)(=O)c1ccc(-c2ccc3c(N)ncnn23)cc1]

7-bromopyrrolo[2,1-f][1,2,4]triazin-4-amine (2.00 g, 9.39 mmol), (4-(methylsulfonyl)phenyl)boronic acid (1.972 g, 9.86 mmol) and PdCl₂(dppf)-CH₂Cl₂ adduct (383 mg, 0.469 mmol) were placed in a round-bottom flask. Then THF (60 mL), water (37.6 mL) and Phosphoric acid, potassium salt (3986 mg, 18.8 mmol) were added, and the reaction mixture was degassed (3×, vacuum/Ar). The flask was equipped a reflux condenser, and the reaction mixture was stirred at 100° C. (reflux) for 4 h. The reaction mixture was cooled to rt, diluted with EtOAc (100 mL), Celite was added and most of the solvent was removed under reduced pressure. The residue was purified by flash chromatography to give Example 1A (2015 mg, 74% yield) as an off-white solid. MS: [M+H]⁺=289.0; ¹H NMR (500 MHz, DMSO-d₆) δ ppm 8.38 (d, J=8.8 Hz, 2H), 8.00 (s, 1H), 7.98 (d, J=8.8 Hz, 2H), 7.89 (br s, 2H), 7.25 (d, J=4.4 Hz, 1H), 7.06 (d, J=4.7 Hz, 1H), 3.24 (s, 3H).

### Example 1B. 5-Bromo-7-(4-(methylsulfonyl)phenyl)pyrrolo[2,1-f][1,2,4]triazin-4-amine

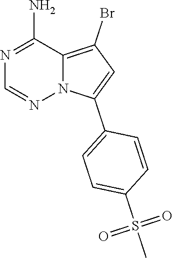 [SMILES: CS(=O)(=O)c1ccc(-c2cc(Br)c3c(N)ncnn23)cc1]

To a solution of Example 1A (500 mg, 1.73 mmol) in DMF (16 mL) at rt, was added NBS (340 mg, 1.91 mmol). The mixture was stirred at rt protected from light for 1 h. The reaction mixture was added into well-stirred 150 mL half-saturated NaHCO₃ and ~10 mL sat. Na₂SO₃ was added. The resultant suspension was stirred at rt for 2 h, then was filtered. The collected solid was rinsed with H₂O (3×), then Et₂O (1×). The solid was dried in vacuo to afford Example 1B (564 mg, 89% yield) as a grey solid. MS: [M+H]⁺=367.0; ¹H NMR (500 MHz, DMSO-d₆) δ ppm 8.36-8.30 (m, 2H), 8.02 (s, 1H), 8.01-7.98 (m, 2H), 7.45 (s, 1H), 3.25 (s, 3H).

### Example 1

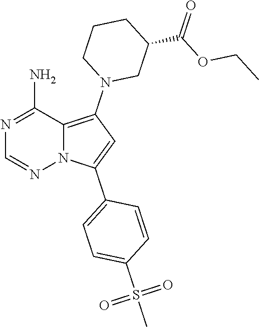 [SMILES: CCOC(=O)[C@H]1CCCN(c2cc(-c3ccc(S(C)(=O)=O)cc3)n3ncnc(N)c23)C1]

Example 1B (15 mg, 0.041 mmol), ethyl (S)-piperidine-3-carboxylate (12.8 mg, 0.082 mmol), Ir[dF(CF₃)ppy]2(dtbbpy)PF₆ (0.46 mg, 0.408 μmol) and DABCO (8.3 mg, 0.074 mmol) were placed in a pressure relief vial. The reaction mixture was degassed (3× vacuum/nitrogen), then a solution of NiBr₂-DME (0.63 mg, 2.04 μmol) in DMA (1.0 mL) was added. The reaction mixture was degassed again, capped and stirred under blue LED irradiation with fan cooling at rt for 20 h. Additional amount of Ir[dF(CF₃)ppy]₂(dtbbpy)PF₆ (0.46 mg, 0.41 μmol) and NiBr₂-DME (0.63 mg, 2.0 μmol) were added. The reaction mixture was degassed again, capped and stirred under blue LED irradiation with fan cooling at rt for 20 h. The reaction mixture was diluted with DMF (1.0 mL), silica-based Pd scavenger was added, and the mixture was stirred at rt for 15 min. The mixture was filtered through a membrane filter, the obtained fraction (2.0 mL) was acidified with TFA (~0.05 mL) and was purified by preparative HPLC to afford Example 1 (3.2 mg, 17% yield). LC-MS Method A: RT=1.680 min, [M+H]⁺=444.0; LC-MS Method B: RT=1.322 min, [M+H]⁺=444.0; ¹H NMR (500 MHz, DMSO-d₆) δ ppm 8.38-8.31 (m, 2H), 8.07-7.96 (m, 1H), 7.95 (d, J=8.7 Hz, 2H), 7.88 (d, J=1.3 Hz, 1H), 7.26 (d, J=1.9 Hz, 1H), 7.23-7.05 (m, 1H), 4.16-4.01 (m, 2H), 3.61-3.54 (m, 8H), 3.22 (d, J=1.3 Hz, 3H), 1.17 (t, J=7.1 Hz, 3H).

### Example 2

### Preparation of N-(1-(4-amino-7-(4-(methylsulfonyl)phenyl)pyrrolo[2,1-f][1,2,4]triazin-5-yl)piperidin-3-yl)picolinamide, TFA

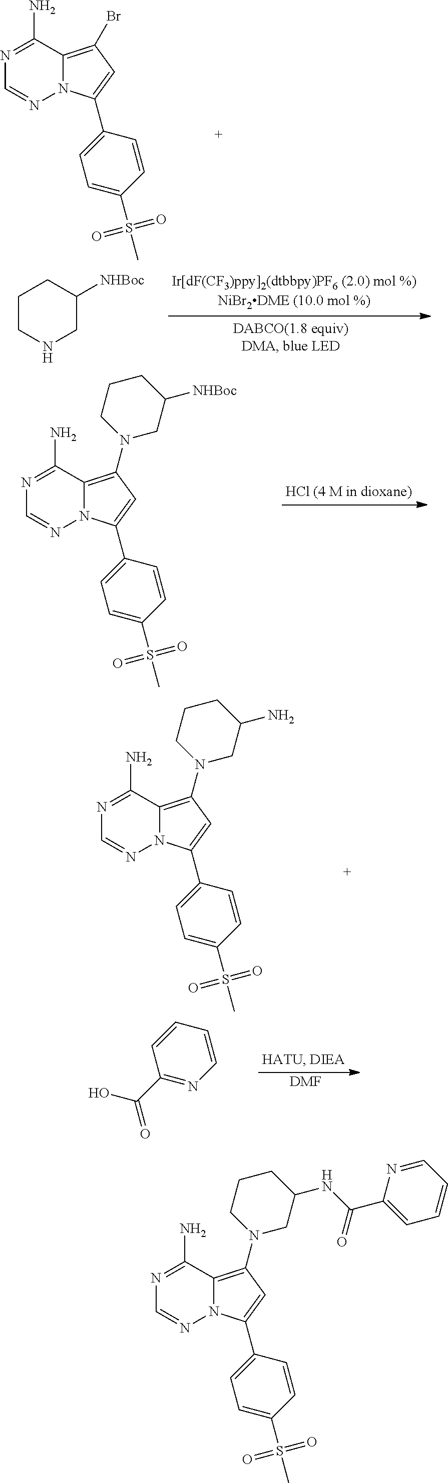

### Example 2a. tert-Butyl (1-(4-amino-7-(4-(methylsulfonyl)phenyl)pyrrolo[2,1-f][1,2,4]triazin-5-yl)piperidin-3-yl)carbamate

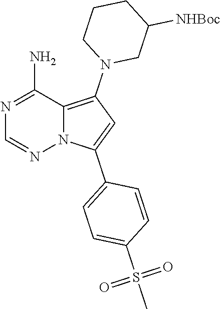 [SMILES: CC(C)(C)OC(=O)NC1CCCN(c2cc(-c3ccc(S(C)(=O)=O)cc3)n3ncnc(N)c23)C1]

Example 1B (150 mg, 0.408 mmol), Ir[dF(CF₃)ppy]₂(dtbbpy)PF₆ (9.2 mg, 8.2 μmol), NiBr₂-DME (12.6 mg, 0.041 mmol) and DABCO (165 mg, 1.47 mmol) were placed in a pressure relief vial. The reaction mixture was degassed (3× vacuum/nitrogen), then a solution of tert-butyl piperidin-3-ylcarbamate (491 mg, 2.45 mmol) in DMA (5.0 mL) was added. The reaction mixture was degassed again, capped and stirred under blue LED irradiation without fan cooling at 50° C. for 3 d. The reaction mixture was diluted with EtOAc (200 mL), washed with water (3×50 mL), brine (1×50 mL), dried (Na₂SO₄) and filtered. EtOAc was removed under reduced pressure, the residue was dissolved in CHCl₃, and was purified by flash chromatography (20-100% EtOAc/DCM) to give Example 2a (97 mg, 49% yield) as a white solid. MS: [M+H]⁺=487.0; ¹H NMR (500 MHz, DMSO-d₆) δ ppm 8.33 (br d, J=8.2 Hz, 2H), 7.94 (br d, J=8.5 Hz, 2H), 7.87 (s, 1H), 7.16 (s, 1H), 7.10-6.92 (m, 1H), 3.68 (br d, J=4.6 Hz, 2H), 3.20 (s, 3H), 3.13-3.02 (m, 1H), 2.95-2.84 (m, 1H), 1.89-1.73 (m, 2H), 1.71-1.56 (m, 1H), 1.35 (s, 9H).

### Example 2b. 5-(3-Aminopiperidin-1-yl)-7-(4-(methylsulfonyl)phenyl)pyrrolo[2,1-f][1,2,4]triazin-4-amine, HCl

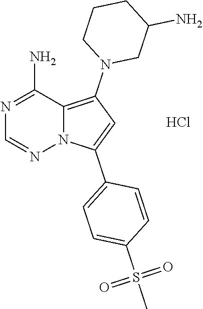 [SMILES: CS(=O)(=O)c1ccc(-c2cc(N3CCCC(N)C3)c3c(N)ncnn23)cc1 ; Cl]

Example 2a (97 mg, 0.199 mmol) was placed in a round-bottom flask. HCl (4 M in dioxane) (5.0 mL, 20.0 mmol) was added. The reaction mixture was stirred at rt for 5 h. Solvent was removed under reduced pressure, the residue was co-evaporated with Et₂O (3×5 mL), and dried under vacuum to afford Example 2b (83 mg, 98% yield) as an off-white solid. MS: [M+H]⁺=387.0; ¹H NMR (500 MHz, DMSO-d₆) δ ppm 8.37-8.32 (m, 2H), 8.27 (br s, 2H), 8.04 (s, 1H), 8.03-7.98 (m, 2H), 7.30 (s, 1H), 3.74-3.70 (m, 3H), 3.69-3.65 (m, 31H), 3.63-3.57 (m, 1H), 3.54-3.50 (m, 2H), 3.49-3.44 (m, 2H), 3.27 (s, 3H).

### Example 2

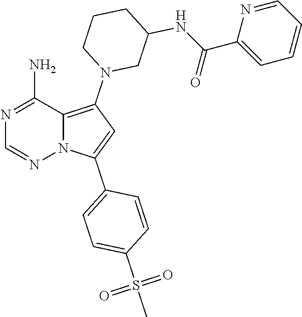 [SMILES: CS(=O)(=O)c1ccc(-c2cc(N3CCCC(NC(=O)c4ccccn4)C3)c3c(N)ncnn23)cc1]

Example 2b (15 mg, 0.035 mmol), picolinic acid (6.6 mg, 0.053 mmol) and DIEA (0.031 mL, 0.18 mmol) were suspended in anhydrous DMF (1.5 mL). Afterwards, HATU (20.2 mg, 0.053 mmol) was added and the reaction mixture was stirred at 80° C. for 16 h. The reaction mixture was quenched with MeOH (0.1 mL), acidified with TFA (0.1 mL), diluted with DMF to 2 mL, filtered and purified by preparative HPLC to afford Example 2 (4.0 mg, 19% yield). LC-MS Method A: RT=1.595 min, [M+H]⁺=492.2; LC-MS Method B: RT=1.315 min, [M+H]⁺=492.2; ¹H NMR (500 MHz, DMSO-d₆) δ ppm 8.69 (br d, J=8.5 Hz, 1H), 8.66 (br d, J=4.6 Hz, 1H), 8.32 (d, J=8.2 Hz, 2H), 8.03 (br t, J=7.6 Hz, 1H), 8.01-7.92 (m, 3H), 7.65-7.57 (m, 1H), 7.26 (s, 1H), 4.31-4.21 (m, 1H), 3.56-3.37 (m, 4H), 3.23 (s, 3H), 3.11-3.02 (m, 1H), 2.94-2.84 (m, 1H), 2.84-2.75 (m, 1H), 1.95-1.83 (m, 3H), 1.73-1.61 (m, 1H).

The following examples in Table 1 were prepared using the same procedure as shown in Example 2. Example 2b was coupled with the appropriate carboxylic acid. Various coupling reagents could be used other than the one described, such as BOP, PyBop, EDC/HOBt or T3P.

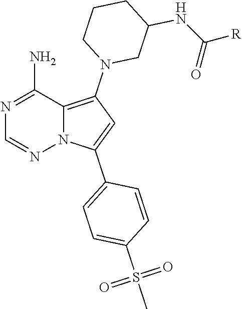

TABLE 1

| | | | | HPLC | |
|---|---|---|---|---|---|
| | | | LCMS | Method, | |
| Example | R | Name | (M + H)⁺ | RT (min.) | ¹H NMR |
| 3 | 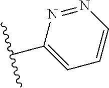 [SMILES: *c1cccnn1] | N-(1-(4-amino-7-(4- (methylsulfonyl)phenyl) pyrrolo[2,1-f][1,2,4]triazin-5- yl)piperidin-3-yl)pyridazine- 3-carboxamide, TFA | 493.1 | A: 1.408 B: 1.183 | (500 MHz, DMSO-d₆) δ ppm 9.46-9.36 (m, 1H), 9.15 (br d, J = 7.6 Hz, 1H), 8.37 (br d, J = 8.5 Hz, 2H), 8.20 (br d, J = 8.9 Hz, 1H), 7.96 (br d, J = 8.9 Hz, 2H), 7.94-7.85 (m, 2H), 7.23 (s, 1H), 4.36-4.25 (m, 1H), 3.36-3.25 (m, 2H), 3.13-3.03 (m, 1H), 2.98-2.84 (m, 1H), 2.82-2.69 (m, 1H), |
| | | | | | 2.01-1.82 (m, 3H), 1.80-1.66 (m, 1H), 1.16 (br t, |
| | | | | | J = 7.2 Hz, 1H) |
| 4 | 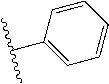 [SMILES: *c1ccccc1] | N-(1-(4-amino-7-(4- (methylsulfonyl)phenyl) pyrrolo[2,1-f][1,2,4]triazin-5- yl)piperidin-3-yl)benzamide | 491.1 | A: 1.578 B: 1.314 | (500 MHz, DMSO-d₆) δ ppm 8.42-8.33 (m, 3H), 8.13-7.99 (m, 1H), 7.99-7.93 (m, 2H), 7.90 (d, J = 3.4 Hz, 1H), 7.83 (br dd, J = 7.6, 2.1 Hz, 2H), 7.56-7.49 (m, 1H), 7.49-7.38 (m, 2H), 7.21 (d, J = 3.4 Hz, 1H), 7.09-6.94 (m, 1H), 4.27-4.14 (m, 1H), 3.22 (br s, 3H), 3.11-3.00 (m, 1H), 2.82-2.66 |
| | | | | | (m, 1H), 2.00-1.86 (m, 2H), 1,85-1.73 (m, 1H), |
| | | | | | 1.61-1.47 (m, 1H) |
| 5 | 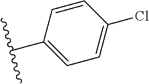 [SMILES: *c1ccc(Cl)cc1] | N-(1-(4-amino-7-(4- (methylsulfonyl)phenyl) pyrrolo[2,1-f][1,2,4]triazin-5- yl)piperidin-3-yl)-4- chlorobenzamide, TFA | 524.9 | A: 1.785 B: 1.545 | (500 MHz, DMSO-d₆) δ ppm 8.49 (br d, J = 7.4 Hz, 1H), 8.35 (br d, J = 8.5 Hz, 2H), 7.97 (br d, J = 8.4 Hz, 2H), 7.94 (br s, 1H), 7.86 (br d, J = 8.2 Hz, 2H), 7.53 (br d, J = 8.3 Hz, 2H), 7.24 (s, 1H), 4.26-4.13 (m, 1H), 3.54-3.41 (m, 1H), 3.23 (s, 3H), 3.11-3.00 (m, 1H), 2.83-2.67 (m, |
| | | | | | 1H), 2.00-1.86 (m, 2H), 1.86-1.76 (m, 1H), |
| | | | | | 1.65-1.47 (m, 1H) |
| 6 | 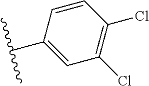 [SMILES: *c1ccc(Cl)c(Cl)c1] | N-(1-(4-amino-7-(4- (methylsulfonyl)phenyl) pyrrolo[2,1-f][1,2,4]triazin-5- yl)piperidin-3-yl)-3,4- dichlorobenzamide | 559.2 | A: 1.960 B: 1.690 | (500 MHz, DMSO-d₆) δ ppm 8.58 (br d, J = 7.3 Hz, 1H), 8.41-8.32 (m, 2H), 8.10 (d. J = 1.8 Hz, 1H), 8.00-7.96 (m, 2H), 7.93 (s, 1H), 7.84 (dd, J = 8.4, 2.0 Hz, 1H), 7.75 (d, J = 8.5 Hz, 1H), 7.24 (s, 1H), 4.26-4.12 (m, 1H), 3.24 (s, 2H), 3.13-3.02 (m, 1H), 2.82-2.70 (m, 1H), 1.99-1.88 |
| | | | | | (m, 2H), 1.87-1.77 (m, 1H), 1.63-1.47 (m, 1H) |
| 7 | 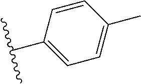 [SMILES: *c1ccc(C)cc1] | N-(1-(4-amino-7-(4- (methylsulfonyl)phenyl) pyrrolo[2,1-f][1,2,4]triazin-5- yl)piperidin-3-yl)-4- methylbenzamide, TFA | 505.9 | A: 1.624 B: 1.379 | (500 MHz, DMSO-d₆) δ ppm 8.32 (ddd, J = 6.3, 4.4, 2.0 Hz, 3H), 8.00-7.98 (m, 1H), 7.96 (d, J = 2.6 Hz, 2H),7.73 (br dd, J = 8.2, 1.2 Hz, 2H), 7.29-7.21 (m, 3H), 4.27-4.14 (m, 1H), 3.30-3.24 (m, 1H), 3.22 (br d, J = 0.9 Hz, 2H), 3.11-3.03 (m, 1H), 2.80-2.67 (m, 2H), 2.33 (s, 3H), |
| | | | | | 1.98-1.78 (m, 3H), 1.60-1.46 (m, 1H) |
| 8 | 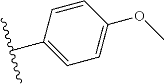 [SMILES: *c1ccc(OC)cc1] | N-(1-(4-amino-7-(4- (methylsulfonyl)phenyl) pyrrolo[2,1-f][1,2,4]triazin-5- yl)piperidin-3-yl)-4- methoxybenzamide, TFA | 521.3 | A: 1.591 B: 1.363 | (500 MHz, DMSO-d₆) δ ppm 8.33 (br d, J = 8.6 Hz, 2H), 8.24 (br d, J = 7.6 Hz, 1H), 8.01-7.93 (m, 3H), 7.85-7.79 (m, 2H), 7.27-7.21 (m, 1H), 6.98 (d, J = 8.8 Hz, 2H), 4.25-4.13 (m, 1H), 3.84-3.74 (m, 3H), 3.59-3.48 (m, 2H), 3.26-3.19 (m, 3H), 3.11-3.03 (m, 1H), 2.82-2.66 (m, 2H), |
| | | | | | 1.96-1.79 (m, 3H), 1.63-1.47 (m, 1H) |

### Example 9

### Preparation of 1-(1-(4-amino-7-(4-(methylsulfonyl)phenyl)pyrrolo[2,1-f][1,2,4]triazin-5-yl)piperidin-3-yl)-3-phenylurea, TFA

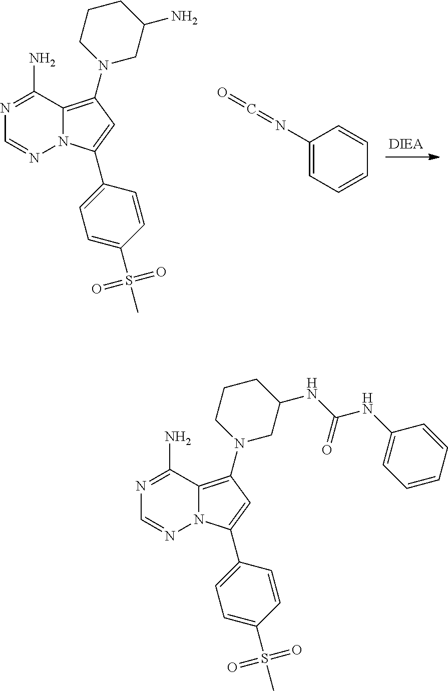 [SMILES: CS(=O)(=O)c1ccc(-c2cc(N3CCCC(N)C3)c3c(N)ncnn23)cc1 ; O=C=Nc1ccccc1 ; CS(=O)(=O)c1ccc(-c2cc(N3CCCC(NC(=O)Nc4ccccc4)C3)c3c(N)ncnn23)cc1]

Example 2B (15 mg, 0.035 mmol) and DIEA (0.031 mL, 0.177 mmol) were suspended in anhydrous THF (1.5 mL). Afterwards, isocyanatobenzene (0.012 mL, 0.106 mmol) was added and the reaction mixture was stirred at rt for 1 h. The reaction mixture was quenched with MeOH (1.0 mL), and the solvent was removed under reduced pressure. The residue was dissolved in DMF (2.0 mL), acidified with TFA (0.1 mL), filtered and purified by preparative HPLC to afford Example 9 (2.6 mg, 10% yield). LC-MS Method A: RT=1.650 min, [M+H]⁺=506.3; LC-MS Method B: RT=1.405 min, [M+H]⁺=506.3; ¹H NMR (500 MHz, DMSO-d₆) δ ppm 8.39 (s, 1H), 8.35 (br d, J=8.5 Hz, 2H), 7.96 (br d, J=8.5 Hz, 2H), 7.91 (s, 1H), 7.37 (br d, J=8.5 Hz, 2H), 7.26-7.17 (m, 4H), 6.93-6.83 (m, 1H), 3.96-3.86 (m, 1H), 3.75-3.62 (m, 1H), 3.22 (s, 3H), 3.05-2.79 (m, 2H), 1.90-1.70 (m, 3H), 1.48-1.35 (m, 1H), 1.29-1.09 (m, 1H).

### Example 11

### Preparation of tert-butyl (R)-(1-(4-amino-7-(4-(methylsulfonyl)phenyl)pyrrolo[2,1-f][1,2,4]triazin-5-yl)piperidin-3-yl)carbamate

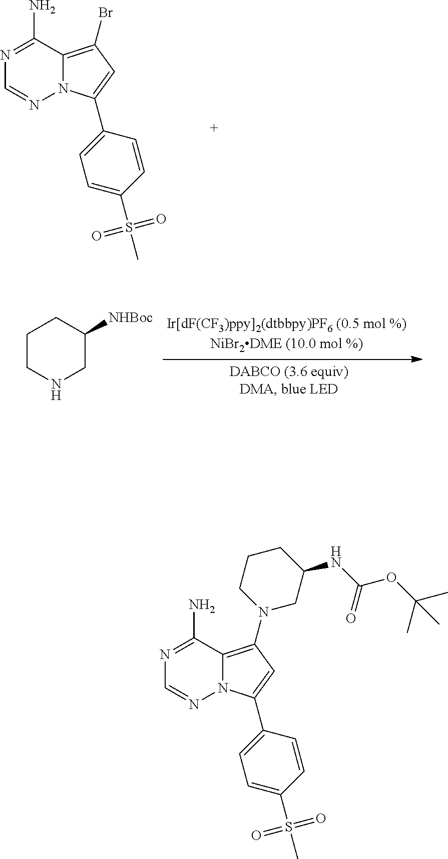 [SMILES: CS(=O)(=O)c1ccc(-c2cc(Br)c3c(N)ncnn23)cc1 ; CC(C)(C)OC(=O)N[C@@H]1CCCNC1 ; CC(C)(C)OC(=O)N[C@@H]1CCCN(c2cc(-c3ccc(S(C)(=O)=O)cc3)n3ncnc(N)c23)C1]

Example 1B (150 mg, 0.408 mmol), Ir[dF(CF₃)ppy]₂(dtbbpy)PF₆ (2.3 mg, 2.04 μmol), tert-butyl (R)-piperidin-3-ylcarbamate (245 mg, 1.23 mmol) and DABCO (165 mg, 1.47 mmol) were placed in a pressure relief vial. The reaction mixture was degassed (3× vacuum/nitrogen), then a solution of NiBr₂-DME (12.6 mg, 0.041 mmol) in DMA (5.0 mL) was added. The reaction mixture was degassed again, capped and stirred under blue LED irradiation without fan cooling at 70° C. for 2 d. The reaction mixture was diluted with EtOAc (200 mL), washed with water (3×50 mL), brine (1×50 mL), dried (Na₂SO₄) and filtered. EtOAc was removed under reduced pressure, the residue was purified by flash chromatography (20-100% EtOAc/DCM gradient) to give Example 11 (120 mg, 60% yield) as a white solid. LC-MS Method A: RT=1.834 min, [M+H]⁺=487.1; LC-MS Method B: RT=1.499 min, [M+H]⁺=; 487.1 ¹H NMR (500 MHz, DMSO-d₆) δ ppm 8.41-8.35 (m, 1H), 8.03-7.97 (m, 1H), 7.96 (d, J=8.9 Hz, 2H), 7.90 (s, 1H), 7.20 (s, 1H), 3.74-3.61 (m, 1H), 3.24 (s, 3H), 3.15-3.05 (m, 1H), 2.96-2.84 (m, 1H), 1.94-1.82 (m, 1H), 1.82-1.74 (m, 1H), 1.72-1.61 (m, 1H), 1.38 (s, 9H), 1.36-1.30 (m, 1H), 1.29-1.21 (m, 2H).

### Example 13

### Preparation of (R)—N-(1-(4-amino-7-(4-(methylsulfonyl)phenyl)pyrrolo[2,1-f][1,2,4]triazin-5-yl)piperidin-3-yl)benzamide

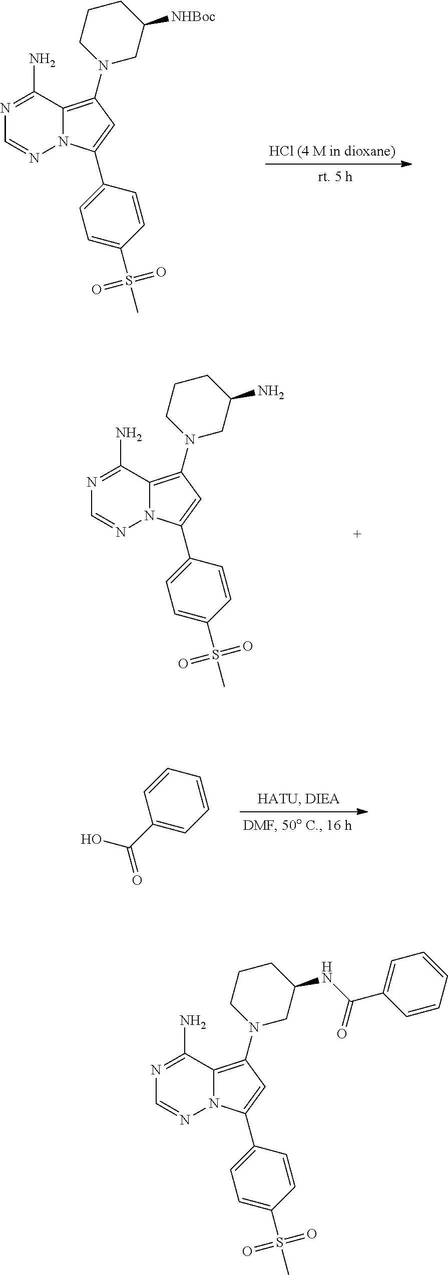 [SMILES: CC(C)(C)OC(=O)N[C@@H]1CCCN(c2cc(-c3ccc(S(C)(=O)=O)cc3)n3ncnc(N)c23)C1 ; CS(=O)(=O)c1ccc(-c2cc(N3CCC[C@@H](N)C3)c3c(N)ncnn23)cc1 ; O=C(O)c1ccccc1 ; CS(=O)(=O)c1ccc(-c2cc(N3CCC[C@@H](NC(=O)c4ccccc4)C3)c3c(N)ncnn23)cc1]

### Example 13A. (R)-5-(3-aminopiperidin-1-yl)-7-(4-(methylsulfonyl)phenyl)pyrrolo[2,1-f][1,2,4]triazin-4-amine, HCl

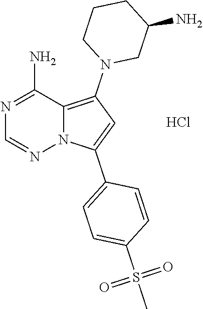 [SMILES: CS(=O)(=O)c1ccc(-c2cc(N3CCC[C@@H](N)C3)c3c(N)ncnn23)cc1 ; Cl]

Example 11 (120 mg, 0.247 mmol) was placed in a round-bottom flask. HCl (4 M in dioxane) (5.0 mL, 20.0 mmol) was added. The reaction mixture was stirred at rt for 5 h. Solvent was removed under reduced pressure, the residue was co-evaporated with Et₂O (3×5 mL), and dried under vacuum to afford Example 13A (75 mg, 72% yield) as an off-white solid. MS: [M+H]⁺=387.2; ¹H NMR (500 MHz, DMSO-d₆) δ ppm 8.37-8.32 (m, 2H), 8.27 (br s, 2H), 8.04 (s, 1H), 8.03-7.98 (m, 2H), 7.30 (s, 1H), 3.74-3.70 (m, 3H), 3.69-3.65 (m, 3H), 3.63-3.57 (m, 1H), 3.54-3.50 (m, 2H), 3.49-3.44 (m, 2H), 3.27 (s, 3H).

### Example 13

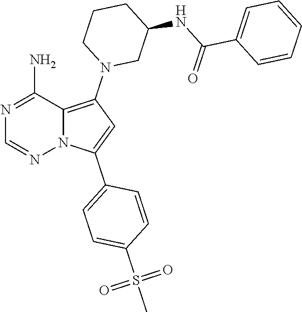 [SMILES: CS(=O)(=O)c1ccc(-c2cc(N3CCC[C@@H](NC(=O)c4ccccc4)C3)c3c(N)ncnn23)cc1]

Example 13A (11 mg, 0.026 mmol), benzoic acid (4.1 mg, 0.034 mmol) and DIEA (0.023 mL, 0.13 mmol) were suspended in anhydrous DMF (1.5 mL). Afterwards, HATU (12.9 mg, 0.034 mmol) was added and the reaction mixture was stirred at 50° C. for 16 h. The reaction mixture was quenched with MeOH (0.1 mL), acidified with TFA (0.1 mL), diluted with DMF, filtered and purified by preparative HPLC to afford Example 13 (1.3 mg, 10% yield). LC-MS Method A: RT=1.514 min, [M+H]⁺=491.3; LC-MS Method B: RT=1.211 min, [M+H]⁺=491.3; ¹H NMR (500 MHz, DMSO-d₆) δ ppm. 8.41-8.37 (m, 1H), 8.36 (d, J=8.5 Hz, 2H), 8.12-7.97 (m, 2H), 7.98-7.92 (m, 2H), 7.89 (s, 1H), 7.85-7.79 (m, 2H), 7.56-7.49 (m, 1H), 7.47-7.41 (m, 2H), 7.21 (s, 1H), 7.11-6.93 (m, 1H), 4.25-4.13 (m, 1H), 3.07-3.00 (m, 1H), 2.79-2.69 (m, 1H), 1.97-1.86 (m, 2H), 1.85-1.75 (m, 1H), 1.62-1.49 (m, 1H).

The following examples in Table 2 were prepared using the same procedure as shown in Example 13. Example 13A was coupled with the appropriate carboxylic acid. Various coupling reagents could be used other than the one described, such as BOP, PyBop, EDC/HOBt or T3P.

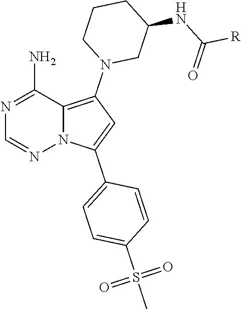

TABLE 2

| | | | | HPLC | |
|---|---|---|---|---|---|
| | | | | Method, | |
| Ex- | Structure | | LCMS | RT | |
| ample | (R) | Name | (M + H)⁺ | (min.) | ¹H NMR |
| 14 | 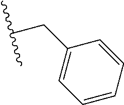 [SMILES: *Cc1ccccc1] | (R)-N-(1-(4-amino-7-(4- (methylsulfonyl)phenyl) pyrrolo[2,1- f][1,2,4]triazin-5- yl)piperidin-3-yl)-2- phenylacetamide, TFA | 505.1 | A: 1.660 B: 1.403 | (500 MHz, DMSO-d₆) δ ppm 8.33 (d, J = 8.5 Hz, 2H), 8.27- 8.19 (m, 1H), 8.00-7.94 (m, 2H), 7.92 (s, 1H), 7.30- 7.22 (m, 4H), 7.20 (s, 1H), 4.00-3.89 (m, 1H), 3.72- 3.64 (m, 1H), 3.22 (s, 3H), 3.12-3.02 (m, 1H), 2.99- 2.91 (m, 1H), 1.93-1.84 (m, 1H), 1.83-1.65 (m, 2H), 1.48-1.33 (m, 1H) |
| 15 | 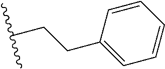 [SMILES: *CCc1ccccc1] | (R)-N-(1-(4-amino-7-(4- (methylsulfonyl)phenyl) pyrrolo[2,1- f][1,2,4]triazin-5- yl)piperidin-3-yl)-3- phenylpropanamide, TFA | 519.1 | A: 1.457 B: 1.178 | (500 MHz, DMSO-d₆) δ ppm 8.35 (d, J = 8.9 Hz, 2H), 7.98 (d, J = 8.5 Hz, 2H), 7.94 (s, 1H), 7.29-7.22 (m, 2H), 7.21- 7.17 (m, 3H), 7.16-7.09 (m, 1H), 4.05-3.92 (m, 1H), 3.24 (s, 3H), 3.09-3.02 (m, 1H), 3.01-2.86 (m, 1H), 2.84-2.75 (m, 2H), 2.45-2.36 (m, 2H), 1.91-1.81 (m, 1H), 1.80-1.64 (m, 2H), 1.43-1.30 (m, 1H) |
| 16 | 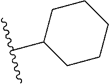 [SMILES: *C1CCCCC1] | (R)-N-(1-(4-amino-7-(4- (methylsulfonyl)phenyl) pyrrolo[2,1- f][1,2,4]triazin-5- yl)piperidin-3- yl) cyclohexanecarboxamide | 497.2 | A: 1.754 B: 1.489 | (500 MHz, DMSO-d₆) δ ppm 8.40-8.31 (m, 2H), 8.14- 7.98 (m, 1H), 7.98-7.93 (m, 2H), 7.90 (s, 1H), 7.85- 7.70 (m, 1H), 7.19 (s, 1H), 7.08-6.88 (m, 1H), 4.00- 3.86 (m, 1H), 3.23 (s, 3H), 3.13-3.04 (m, 1H), 3.01- 2.90 (m, 1H), 2.19-2.06 (m, 1H), 1.94-1.83 (m, 1H), 1.81-1.74 (m, 1H), 1.67 (br d, J = 11.3 Hz, 4H), 1.64- 1.54 (m, 2H), 1.45-1.26 (m, 3H), 1.25-1.02 (m, 3H) |
| 17 | 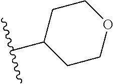 [SMILES: *C1CCOCC1] | (R)-N-(1-(4-amino-7-(4- (methylsulfonyl)phenyl) pyrrolo[2,1- f][1,2,4]triazin-5- yl)piperidin-3- yl)tetrahydro-2H-pyran- 4-carboxamide, TFA | 499.3 | A: 1.326 B: 1.108 | (500 MHz, DMSO-d₆) δ ppm 8.39-8.31 (m, 2H), 7.97 (d, J = 8.5 Hz, 2H), 7.93 (s, 1H), 7.91-7.85 (m, 1H), 7.22 (s, 1H), 3.96 (ddt, J = 6.8, 3.2, 1.5 Hz, 1H), 3.89-3.77 (m, 2H), 3.35-3.25 (m, 1H), 3.23 (s, 3H), 3.13-3.03 (m, 1H), 3.01-2.87 (m, 1H), 2.44-2.33 (m, 1H), 1.94-1.84 (m, 1H), 1.81-1.67 (m, 2H), 1.62-1.51 (m, 4H), 1.48- 1.31 (m, 1H) |
| 19 | 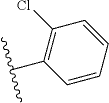 [SMILES: *c1ccccc1Cl] | (R)-N-(1-(4-amino-7-(4- (methylsulfonyl)phenyl) pyrrolo[2,1- f][1,2,4]triazin-5- yl)piperidin-3-yl)-2- chlorobenzamide | 525.0 | A: 1.692 B: 1.430 | (500 MHz, DMSO-d₆) δ ppm 8.69-8.49 (m, 1H), 8.34 (dt, J = 8.7, 1.9 Hz, 2H), 7.95 (dt, J = 8.6, 1.5 Hz, 2H), 7.91- 7.84 (m, 1H), 7.51-7.44 (m, 1H), 7.45-7.32 (m, 3H), 7.25-7.17 (m, 1H), 7.12-6.98 (m, 1H), 4.25-4.12 (m, 1H), 3.82 (s, 1H), 3.65-3.59 (m, 2H), 3.27-3.17 (m, 3H), 1,99-1.81 (m, 2H), 1.81-1.66 (m, 1H), 1.58-1.30 (m, 1H) |
| 20 | 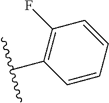 [SMILES: *c1ccccc1F] | (R)-N-(1-(4-amino-7-(4- (methylsulfonyl)phenyl) pyrrolo[2,1- f][1,2,4]triazin-5- yl)piperidin-3-yl)-2- fluorobenzamide | 509.3 | A: 1.647 B: 1.385 | (500 MHz, DMSO-d₆) δ ppm 8.41 (ddt, J = 8.4, 3.5, 1.6 Hz, 1H), 8.37 (d, J = 8.5 Hz, 2H), 8.12-7.99 (m, 1H), 7.96 (d, J = 8.5 Hz, 2H), 7.91 (s, 1H), 7.56 (td, J = 7.2, 1.7 Hz, 1H), 7.54-7.44 (m, 1H), 7.30-7.24 (m, 2H), 7.23 (s, 1H), 7.13-6.88 (m, 1H), 4.26-4.13 (m, 1H), 3.23 (s, 3H), 2.85-2.68 (m, 1H), 1.97-1.85 (m, 2H), 1.85-1.70 (m, 1H), 1.62-1.44 (m, 1H) |
| 21 | 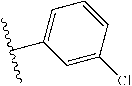 [SMILES: *c1cccc(Cl)c1] | (R)-N-(1-(4-amino-7-(4- (methylsulfonyl)phenyl) pyrrolo[2,1- f][1,2,4]triazin-5- yl)piperidin-3-yl)-3- chlorobenzamide | 525.3 | A: 1.770 B: 1.507 | (500 MHz, DMSO-d₆) δ ppm 8.51 (br d, J = 8.2 Hz, 1H), 8.37 (d, J = 8.5 Hz, 2H), 8.14-8.01 (m, 1H), 7.96 (d, J = 8.9 Hz, 2H), 7.92-7.87 (m, 2H), 7.80 (d, J = 8.2 Hz, 1H), 7.59 (dd, J = 8.1, 1.4 Hz, 1H), 7.53-7.44 (m, 1H), 7.22 (s, 1H), 4.28-4.12 (m, 1H), 3.23 (s, 3H), 3.10-3.01 (m, 1H), 2.81-2.67 (m, 1H), 2.00-1.87 (m, 2H), 1.87-1.76 (m, 1H), 1.63-1.48 (m, 1H) |
| 22 | 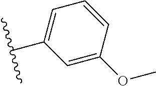 [SMILES: *c1cccc(OC)c1] | (R)-N-(1-(4-amino-7-(4- (methylsulfonyl)phenyl) pyrrolo[2,1- f][1,2,4]triazin-5- yl)piperidin-3-yl)-3- methoxybenzamide | 521.3 | A: 1.625 B: 1.608 | (500 MHz, DMSO-d₆) δ ppm 8.36 (br d, J = 8.9 Hz, 3H), 8.09-7.99 (m, 1H), 7.95 (br d, J = 8.5 Hz, 2H), 7.90 (s, 1H), 7.45-7.40 (m, 1H), 7.39-7.32 (m, 2H), 7.21 (s, 1H), 7.08 (br dd, J = 7.9, 1.5 Hz, 1H), 7.05-6.91 (m, 1H), 4.24-4.12 (m, 1H), 3.79 (s, 3H), 3.22 (s, 3H), 3.10-3.01 (m, 1H), 2.80-2.69 (m, 1H), 2.00-1.86 (m, 2H), 1.86- 1.74 (m, 1H), 1.61-1,47 (m, 1H) |
| 23 | 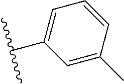 [SMILES: *c1cccc(C)c1] | (R)-N-(1-(4-amino-7-(4- (methylsulfonyl)phenyl) pyrrolo[2,1- f][1,2,4]triazin-5- yl)piperidin-3-yl)-3- methylbenzamide | 505.1 | A: 1.704 B: 1.686 | (500 MHz, DMSO-d₆) δ ppm 8.37 (d, J = 8.5 Hz, 2H), 8.32 (br d, J = 7.6 Hz, 1H), 8.20-8.04 (m, 1H), 7.96 (d, J = 8.5 Hz, 2H), 7.92 (s, 1H), 7.65 (s, 1H), 7.63 (dd, J = 5.0, 3.8 Hz, 1H), 7.32 (br d, J = 0.9 Hz, 2H), 7.22 (s, 1H), 7.14- 6.96 (m, 1H), 4.27-4.13 (m, 1H), 3.23 (s, 3H), 3.11- 3.01 (m, 1H), 2.80-2.67 (m, 1H), 2.35 (s, 3H), 1.98- 1.86 (m, 2H), 1.86-1.76 (m, 1H), 1.63-1.47 (m. 1H) |
| 24 | 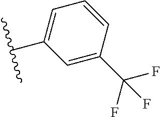 [SMILES: *c1cccc(C(F)(F)F)c1] | (R)-N-(1-(4-amino-7-(4- (methylsulfonyl)phenyl) pyrrolo[2,1- f][1,2,4]triazin-5- yl)piperidin-3-yl)-3- (trifluoromethyl) benzamide | 559.3 | A: 1.831 B: 1.847 | (500 MHz, DMSO-d₆) δ ppm 8.65 (br d, J = 7.6 Hz, 1H). 8.43-8.33 (m, 2H), 8.18 (s, 1H), 8.15 (d, J = 7.6 Hz, 1H), 8.09-8.00 (m, 1H), 7.96 (d, J = 8.5 Hz, 2H), 7.90 (s, 1H), 7.72 (t, J = 7.8 Hz, 1H), 7.22 (s, 1H), 7.10-6.92 (m, 1H), 4.30-4.16 (m, 1H), 3.22 (s, 3H), 3.11-3.05 (m, 1H), 2.84-2.68 (m, 1H), 2.00-1,94 (m, 1H), 1.92-1.76 (m, 2H), 1.63-1.48 (m, 1H) |
| 25 | 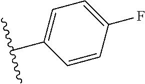 [SMILES: *c1ccc(F)cc1] | (R)-N-(1-(4-amino-7-(4- (methylsulfonyl)phenyl) pyrrolo[2,1- f][1,2,4]triazin-5- yl)piperidin-3-yl)-4- fluorobenzamide | 509.3 | A: 1.831 B: 1.847 | (500 MHz, DMSO-d₆) δ ppm 8.41 (br d, J = 7.6 Hz, 1H), 8.35 (d, J = 8.5 Hz, 2H), 7.95 (d. J = 8.5 Hz, 2H), 7.93-7.84 (m, 3H), 7.27 (t, J = 8.9 Hz, 2H), 7.21 (s, 1H), 4.24-4.11 (m, 1H), 3.22 (s, 3H), 3.10-3.00 (m, 1H), 2.79-2.69 (m, 1H), 1.97-1.86 (m, 2H), 1.85-1.73 (m, 1H), 1.62-1.47 (m, 1H) |
| 26 | 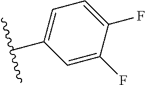 [SMILES: *c1ccc(F)c(F)c1] | (R)-N-(1-(4-amino-7-(4- (methylsulfonyl)phenyl) pyrrolo [2,1- f][1,2,4]triazin-5- yl)piperidin-3-yl)-3,4- difluorobenzamide | 527.2 | A: 1.732 B: 1.460 | (500 MHz, DMSO-d₆) δ ppm 8.47 (br d, J = 7.6 Hz, 1H), 8.40-8.31 (m, 2H), 8.09-8.00 (m, 1H), 7.98-7.93 (m, 2H), 7.89 (s, 1H), 7.78-7.70 (m, 1H), 7.52 (dt, J = 10.5, 8.3 Hz, 1H), 7.21 (s, 1H), 7.09-6.90 (m, 1H), 4.17 (td, J = 5.3, 3.5 Hz, 1H), 3.22 (s, 3H), 3.08-3.00 (m, 1H), 2.80- 2.67 (m, 1H), 2.01-1.85 (m, 2H), 1.86-1.72 (m, 1H), 1.60-1.45 (m, 1H) |
| 27 | 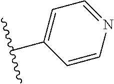 [SMILES: *c1ccncc1] | (R)-N-(1-(4-amino-7-(4- (methylsulfonyl)phenyl) pyrrolo[2,1- f][1,2,4]triazin-5- yl)piperidin-3- yl)isonicotinamide | 492.3 | A: 1.320 B: 1.336 | (500 MHz, DMSO-d₆) δ ppm 8.72 (br d, J = 6.1 Hz, 2H), 8.68 (br d, J = 7.3 Hz, 1H), 8.40-8.33 (m, 2H), 8.24-8.07 (m, 1H), 7.97 (d, J = 8.5 Hz, 2H), 7.92 (s, 1H), 7.75 (d, J = 6.1 Hz, 2H), 7.24 (s, 1H), 7.15-7.00 (m, 1H), 4.29- 4.14 (m, 1H), 3.23 (s, 3H), 3.10-3.01 (m, 1H), 2.85- 2.68 (m, 1H), 1.99-1.88 (m, 2H), 1.88-1.73 (m, 1H), 1.65-1.49 (m, 1H) |
| 28 | 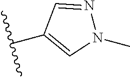 [SMILES: *c1cnn(C)c1] | (R)-N-(1-(4-amino-7-(4- (methylsulfonyl)phenyl) pyrrolo[2,1- f][1,2,4]triazin-5- yl)piperidin-3-yl)-1- methyl-1H-pyrazole-4- carboxamide | 495.1 | A: 1.309 B: 1.109 | (500 MHz, DMSO-d₆) δ ppm 8.36 (d, J = 8.9 Hz, 2H), 8.29- 8.17 (m, 1H), 8.13 (s, 1H), 7.99-7.95 (m, 2H), 7.93 (s, 2H), 7.85 (s, 1H), 7.23 (s, 1H), 4.19-4.09 (m, 1H), 3.84 (s, 3H), 3.23 (s, 3H), 3.05 (dt, J = 2.7, 1.3 Hz, 1H), 2.80- 2.63 (m, 1H), 1.96-1.86 (m, 2H), 1.84-1.72 (m, 1H), 1.56-1.42 (m, 1H) |
| 29 | 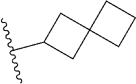 [SMILES: *C1CC2(CCC2)C1] | (R)-N-(1-(4-amino-7-(4- (methylsulfonyl)phenyl) pyrrolo [2,1- f][1,2,4]triazin-5- yl)piperidin-3- yl)spiro[3.3]heptane-2- carboxamide | 509.1 | A: 1.802 B: 1.551 | (500 MHz, DMSO-d₆) δ ppm 8.37 (d, J = 8.5 Hz, 2H), 8.13- 8.00 (m, 1H), 7.96 (d, J = 8.5 Hz, 2H), 7.90 (s, 1H), 7.80- 7.67 (m, 1H), 7.20 (s, 1H), 7.09-6.84 (m, 1H), 3.99- 3.86 (m, 1H), 3.24 (s, 3H), 2.87 (br s, 1H), 2.12-2.02 (m, 3H), 1.99 (br t, J = 7.3 Hz, 2H), 1.91-1.81 (m, 3H), 1.78- 1.62 (m, 3H), 1.43-1.29 (m, 1H) |
| 30 | 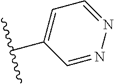 [SMILES: *c1ccnnc1] | (R)-N-(1-(4-amino-7-(4- (methylsulfonyl)phenyl) pyrrolo[2,1- f][1,2,4]triazin-5- yl)piperidin-3- yl)pyridazine-4- carboxamide | 492.9 | A: 1.205 B: 0.939 | (500 MHz, DMSO-d₆) δ ppm 9.50 (s, 1H), 9.41 (d, J = 4.3 Hz, 1H), 8,94 (br d, J = 7.3 Hz, 1H), 8.32 (d, J = 8.5 Hz, 2H), 8.03-7.91 (m, 4H), 7.23 (s, 1H), 4.30-4.15 (m, 1H), 3.22 (s, 3H), 3.10-2.98 (m, 1H), 2.87-2.71 (m, 1H), 2.03-1.87 (m, 2H), 1.87-1.76 (m, 1H), 1.65-1.47 (m, 1H) |
| 31 | 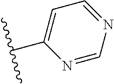 [SMILES: *c1ccncn1] | (R)-N-(1-(4-amino-7-(4- (methylsulfonyl)phenyl) pyrrolo[2,1- f][1,2,4]triazin-5- yl)piperidin-3- yl)pyrimidine-4- carboxamide | 493.1 | A: 1.438 B: 1.229 | (500 MHz, DMSO-d₆) δ ppm 9.33 (s, 1H), 9.05 (d, J = 5.2 Hz, 1H), 8.91 (br d, J = 8.5 Hz, 1H), 8.34 (d, J = 8.9 Hz, 2H), 8.00 (dd, J = 5.2, 1.2 Hz, 1H), 7.97-7.92 (m, 2H), 7.89 (s, 1H), 7.21 (s, 1H), 4.30-4.15 (m, 1H), 3.64-3.41 (m, 2H), 3.21 (s, 3H), 3.19-3.13 (m, 1H), 3.08-2.98 (m, 1H), 2.95-2.70 (m, 1H), 1.95-1,76 (m, 3H), 1.73-1.60 (m, 1H) |
| 32 | 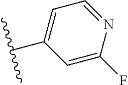 [SMILES: *c1ccnc(F)c1] | (R)-N-(1-(4-amino-7-(4- (methylsulfonyl)phenyl) pyrrolo[2,1- f][1,2,4]triazin-5- yl)piperidin-3-yl)-2- fluoroisonicotinamide, TFA | 510.2 | A: 1.553 B: 1.293 | (500 MHz, DMSO-d₆) δ ppm 8.76 (br d, J = 7.3 Hz, 1H), 8.38 (br d, J = 5.5 Hz, 1H), 8.34 (br d, J = 8.5 Hz, 2H), 8.06- 7.91 (m. 3H), 7.76-7.68 (m, 1H), 7.52 (s, 1H), 7.25 (s, 1H), 4.30-4.15 (m, 1H), 3.30-3.20 (m, 3H), 3.09-3.02 (m, 1H), 2.86-2.68 (m, 1H), 2.01-1.77 (m, 3H), 1.64- 1.47 (m, 1H) |
| 33 | 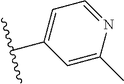 [SMILES: *c1ccnc(C)c1] | (R)-N-(1-(4-amino-7-(4- (methylsulfonyl)phenyl) pyrrolo[2,1- f][1,2,4]triazin-5- yl)piperidin-3-yl)-2- methylisonicotinamide | 506.2 | A: 1.462 B: 1.068 | (500 MHz, DMSO-d₆) δ ppm 8.70-8.62 (m, 1H), 8.59 (d, J = 4.9 Hz, 1H), 8.36 (br d, J = 8.2 Hz, 2H), 7.97 (br d, J = 8.5 Hz, 2H), 7.93 (s, 1H), 7.65 (d, J = 0.6 Hz, 1H), 7.58 (br d, J = 5.5 Hz, 1H), 7.23 (s, 1H), 4.28-4.15 (m, 1H), 3.23 (s, 2H), 3.05 (dt, J = 6.3, 2.5 Hz, 1H), 2.83-2.70 (m, 1H), 2.55 (br s, 3H), 2.54 (br s, 3H), 1.98-1,87 (m, 2H), 1.87- 1.74 (m, 1H), 1.62-1.48 (m, 1H) |
| 34 | 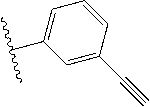 [SMILES: *c1cccc(C#C)c1] | (R)-N-(1-(4-amino-7-(4- (methylsulfonyl)phenyl) pyrrolo[2,1- f][1,2,4]triazin-5- yl)piperidin-3-yl)-3- ethynylbenzamide | 515.2 | A: 1.817 B: 1.562 | (500 MHz, DMSO-d₆) δ ppm 8.49 (br d, J = 8.2 Hz, 1H), 8.37 (br d, J = 8.5 Hz, 2H), 7.99-7.94 (m, 2H), 7.93 (br d, J = 0.9 Hz, 1H), 7.87 (br d, J = 8.2 Hz, 1H), 7.66-7.59 (m, 1H), 7.48 (t, J = 7.9 Hz, 1H), 7.23 (s, 1H), 7.09-6.93 (m, 1H), 4.27 (s, 1H), 4.25-4.14 (m, 1H), 3.23 (s, 1H), 3.13- 2.99 (m, 1H), 2.80-2.64 (m, 1H), 2.00-1.76 (m, 3H), 1.63-1.46 (m, 1H) |
| 35 | 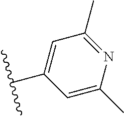 [SMILES: *c1cc(C)nc(C)c1] | (R)-N-(1-(4-amino-7-(4- (methylsulfonyl)phenyl) pyrrolo[2,1- f][1,2,4]triazin-5- yl)piperidin-3-yl)-2,6- dimethylisonicotinamide | 520.2 | A: 1.559 B: 1.124 | (500 MHz, DMSO-d₆) δ ppm 8.58 (br s, 1H), 8.39 (d, J = 8.5 Hz, 2H), 7.99 (d, J = 8.5 Hz, 2H), 7.93 (s, 1H), 7.41 (s, 2H), 7.24 (s, 1H), 7.10-6.94 (m, 1H), 4.28-4.13 (m, 1H), 3.26 (s, 3H), 3.11-3.04 (m, 1H), 2.83-2.72 (m, 1H), 2.55-2.52 (m, 6H), 1.98-1.89 (m, 2H), 1.89-1.78 (m. 1H), 1.65-1.49 (m, 1H) |
| 36 | 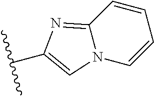 [SMILES: *c1cn2ccccc2n1] | (R)-N-(1-(4-amino-7-(4- (methylsulfonyl)phenyl) pyrrolo[2,1- f][1,2,4]triazin-5- yl)piperidin-3- yl)imidazo[1,2- alpyridine-2- carboxamide | 531.1 | A: 1.450 B: 0.997 | (500 MHz, DMSO-d₆) δ ppm 8.55 (br d, J = 6.7 Hz, 1H), 8.37-8.32 (m, 3H), 8.23 (br d, J = 8.2 Hz, 1H), 7.94 (br d, J = 8.5 Hz, 2H), 7.88 (s, 1H), 7.60 (br d, J = 9.2 Hz, 1H), 7.34 (br dd, J = 8.5, 7.3 Hz, 1H), 7.21 (s, 1H), 6.97 (br t, J = 6.7 Hz, 1H), 4.26-4.16 (m, 1H), 3.64-3.52 (m, 2H), 3.20 (s, 3H), 3.08-2.96 (m, 1H), 2.91-2.71 (m, 1H), 1.96-1.84 (m, 2H), 1.78 (s, 1H), 1.67-1.55 (m, 1H) |
| 37 | 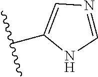 [SMILES: *c1cnc[nH]1] | (R)-N-(1-(4-amino-7-(4- (methylsulfonyl)phenyl) pyrrolo[2,1- f][1,2,4]triazin-5- yl)piperidin-3-yl)-1H- imidazole-5- carboxamide, TFA | 481.2 | A: 1.262 B: 1.017 | (500 MHz, DMSO-d₆) δ ppm 8.72-8.62 (m, 1H), 8.53- 8.43 (m, 1H), 8.37 (br d, J = 8.5 Hz, 2H), 8.06 (br s, 1H), 8.03-7.96 (m, 3H), 7.27 (s, 1H), 4,32-4.18 (m, 1H), 3.61-3.48 (m, 1H), 3.27 (s, 3H), 3.14-3.05 (m, 1H), 2.90-2.73 (m, 2H), 2.58 (s, 3H), 2.01-1.82 (m, 3H), 1.65-1.51 (m, 1H) |
| 38 | 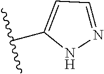 [SMILES: *c1ccn[nH]1] | (R)-N-(1-(4-amino-7-(4- (methylsulfonyl)phenyl) pyrrolo[2,1- f][1,2,4]triazin-5- yl)piperidin-3-yl)-1H- pyrazole-5-carboxamide | 481.0 | A: 1.370 B: 1.164 | (500 MHz, DMSO-d₆) δ ppm 8.36 (br d, J = 8.9 Hz, 2H), 8.13-7.99 (m, 1H), 7.95 (br d, J = 8.5 Hz, 2H), 7.90 (s, 1H), 7.79-7.69 (m, 1H), 7.20 (s, 1H), 6.72-6,64 (m, 1H), 4.23-4.10 (m, 1H), 3.22 (s, 3H), 3.08-3.00 (m, 1H), 2.83-2.69 (m, 1H), 1.95-1.76 (m, 3H), 1.65-1.52 (m, 1H) |
| 39 | 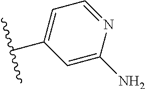 [SMILES: *c1ccnc(N)c1] | (R)-2-amino-N-(1-(4- amino-7-(4- (methylsulfonyl)phenyl) pyrrolo [2,1- f][1,2,4]triazin-5- yl)piperidin-3- yl)isonicotinamide | 507.3 | A: 1.253 B: 1.062 | (500 MHz, DMSO-d₆) δ ppm 8.91-8.83 (m, 1H), 8.40- 8.31 (m, 2H), 8.08-8.02 (m, 1H), 8.00-7.91 (m, 3H), 7.28-7.21 (m, 3H), 7.15 (d, J = 1.8 Hz, 1H), 7.10 (br d, J = 6.3 Hz, 1H), 7.05 (br d, J = 1.4 Hz, 1H), 4.27-4.15 (m, 1H), 3.26-3.20 (m, 3H), 3.09-3.01 (m, 1H), 2.84-2.70 (m, 1H), 1.98-1.87 (m, 2H), 1.87-1.75 (m, 1H), 1.63- 1.48 (m, 1H) |
| 40 | 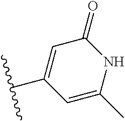 [SMILES: *c1cc(C)[nH]c(=O)c1] | (R)-N-(1-(4-amino-7-(4- (methylsulfonyl)phenyl) pyrrolo[2,1- f][1,2,4]triazin-5- yl)piperidin-3-yl)-6- methyl-2-oxo-1,2- d1Hydropyridine-4- carboxamide, TFA | 522.1 | A: 1.214 B: 1.138 | (500 MHz, DMSO-d₆) δ ppm 8.48 (br d, J = 7.3 Hz, 1H), 8.33 (br d, J = 8.5 Hz, 2H), 8.04-7.89 (m, 3H), 7.23 (s, 1H), 6.52 (br d, J = 0.6 Hz, 1H), 6.27 (s, 1H), 4.42-4.31 (m, 1H), 4.22-4.08 (m, 1H), 3.77-3.73 (m, 1H), 3.23 (s, 3H), 3.09-2.97 (m, 1H), 2.80-2.67 (m, 1H), 2.19 (s, 3H), 2.00-1.71 (m, 3H), 1.62-1.41 (m, 1H) |
| 41 | 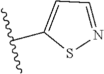 [SMILES: *c1ccns1] | (R)-N-(1-(4-amino-7-(4- (methylsulfonyl)phenyl) pyrrolo[2,1- f][1,2,4]triazin-5- yl)piperidin-3- yl)isothiazole-5- carboxamide, TFA | 498.3 | A: 1.513 B: 1.259 | (500 MHz, DMSO-d₆) δ ppm 8.80 (br d, J = 7.6 Hz, 1H), 8.65 (d, J = 1.8 Hz, 1H), 8.39-8.30 (m, 2H), 8.06-7.91 (m, 4H), 7.24 (s, 1H), 4.31-4.10 (m, 1H), 3.24 (s, 3H), 3.13-3.02 (m, 1H), 2.81-2.70 (m, 1H), 2.01-1.80 (m, 3H), 1.61-1.47 (m, 1H) |
| 42 | 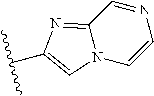 [SMILES: *c1cn2ccncc2n1] | (R)-N-(1-(4-amino-7-(4- (methylsulfonyl)phenyl) pyrrolo 2,1- f][1,2,4]triazin-5- yl)piperidin-3- yl)imidazo[1,2- alpyrazine-2- carboxamide, TFA | 532.1 | A: 1.414 B: 1.198 | (500 MHz, DMSO-d₆) δ ppm 9.14 (s, 1H), 8.66-8.58 (m, 1H), 8.52 (s, 1H), 8.49 (br d, J = 8.5 Hz, 1H), 8.33 (d, J = 8.9 Hz, 2H), 8.01-7.93 (m, 4H), 7.32-7.21 (m, 1H), 4.37-4.19 (m, 1H), 3.23 (s, 3H), 3.10-3.01 (m, 1H), 2.95-2.73 (m, 2H), 1.99-1.80 (m, 3H), 1.77-1.61 (m, 1H) |
| 43 | 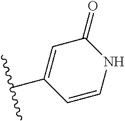 [SMILES: *c1cc[nH]c(=O)c1] | (R)-N-(1-(4-amino-7-(4- (methylsulfonyl)phenyl) pyrrolo[2,1- f][1,2,4]triazin-5- yl)piperidin-3-yl)-2-oxo- 1,2-d1Hydropyridine-4- carboxamide, TFA | 508.0 | A: 1.272 B: 1.095 | (500 MHz, DMSO-d₆) δ ppm 8.52 (br s, 1H), 8.36 (d, J = 8.5 Hz, 2H), 7.96 (d, J = 8.5 Hz, 2H), 7.92 (s, 1H), 7.44 (d, J = 6.7 Hz, 1H), 7.22 (s, 1H), 6.73 (d, J = 1.2 Hz, 1H), 6.45 (dd, J = 6.9, 1.4 Hz, 1H), 4.23-4.06 (m, 1H), 3.23 (s, 3H), 3.06-2.96 (m, 1H), 2.81-2.65 (m, 1H), 1.96-1.71 (m, 3H), 1.60-1.46 (m, 1H) |
| 44 | 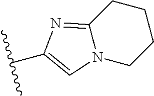 [SMILES: *c1cn2c(n1)CCCC2] | (R)-N-(1-(4-amino-7-(4- (methylsulfonyl)phenyl) pyrrolo[2,1- f][1,2,4]triazin-5- yl)piperidin-3-yl)- 5,6,7,8- tetrahydroimidazo[1,2- alpyridine-2- carboxamide | 534.9 | A: 1.512 B: 1.077 | (500 MHz, DMSO-d₆) δ ppm 8.36 (br d, J = 8.5 Hz, 2H), 7.95 (br d, J = 8.5 Hz, 2H), 7.90 (s, 1H), 7.68 (br d, J = 7.3 Hz, 1H), 7.48 (s, 1H), 7.20 (s, 1H), 4.18-4.07 (m, 1H), 3.96 (br t, J = 5.6 Hz, 2H), 3.22 (s, 3H), 2.73 (brt, J = 6.0 Hz, 3H), 1.95-1.76 (m, 7H), 1.64-1.50 (m, 1H) |
| 45 | 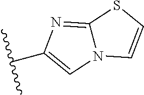 [SMILES: *c1cn2ccsc2n1] | (R)-N-(1-(4-amino-7-(4- (methylsulfonyl)phenyl) pyrrolo [2,1- f][1,2,4]triazin-5- yl)piperidin-3- yl)imidazo[2,1- b]thiazole-6- carboxamide | 537.2 | A: 1.453 B: 1.168 | (500 MHz, DMSO-d₆) δ ppm 8.35 (br d, J = 8.9 Hz, 2H), 8.19 (s, 1H), 8.05 (br d, J = 8.8 Hz, 1H), 7.98-7.91 (m, 3H), 7.88 (s, 1H), 7.35 (d, J = 4.6 Hz, 1H), 7.20 (s, 1H), 7.09-6.80 (m, 1H), 4.24-4.09 (m, 1H), 3.21 (s, 2H), 3.19-3.10 (m, 1H), 3.08-2.95 (m, 1H), 2.87-2.68 (m, 1H), 1.94-1.71 (m, 3H), 1.67-1.54 (m, 1H) |
| 46 | 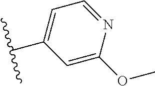 [SMILES: *c1ccnc(OC)c1] | (R)-N-(1-(4-amino-7-(4- (methylsulfonyl)phenyl) pyrrolo[2,1- f][1,2,4]triazin-5- yl)piperidin-3-yl)-2- methoxyisonicotinamide, TFA | 522.2 | A: 1.479 B: 1.183 | (500 MHz, DMSO-d₆) δ ppm 8.62 (br d, J = 7.9 Hz, 1H), 8.33 (d, J = 8.9 Hz, 2H), 8.27 (d, J = 5.5 Hz, 1H), 8.02-7.93 (m, 3H), 7.36-7.29 (m, 1H), 7.24 (s, 1H), 7.17 (s, 1H), 4.29-4.16 (m, 1H), 3.88 (s, 3H), 3.23 (s, 3H), 3.09-3.01 (m, 1H), 2.83-2.68 (m, 2H), 1,98-1.75 (m, 3H), 1.63- 1.45 (m, 1H) |
| 47 | 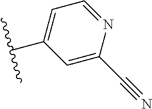 [SMILES: *c1ccnc(C#N)c1] | (R)-N-(1-(4-amino-7-(4- (methylsulfonyl)phenyl) pyrrolo[2,1- f][1,2,4]triazin-5- yl)piperidin-3-yl)-2- cyanoisonicotinamide, TFA | 517.0 | A: 1.512 B: 1.262 | (500 MHz, DMSO-d₆) δ ppm 8.95-8.82 (m, 2H), 8.40- 8.29 (m, 3H), 8.05 (br dd, J = 5.0, 1.6 Hz, 1H), 8.01-7.90 (m, 3H), 7.24 (s, 1H), 4.30-4.18 (m, 1H), 3.71-3.55 (m, 1H), 3.32-3.20 (m, 3H), 3.11-3.01 (m, 1H), 2.87-2.72 (m, 1H), 2.00-1.77 (m, 3H), 1.66-1.49 (m, 1H) |
| 48 | 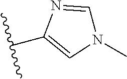 [SMILES: *c1cn(C)cn1] | (R)-N-(1-(4-amino-7-(4- (methylsulfonyl)phenyl) pyrrolo[2,1- f][1,2,4]triazin-5- yl)piperidin-3-yl)-1- methyl-1H-imidazole-4- carboxamide | 495.2 | A: 1.320 B: 1.048 | (500 MHz, DMSO-d₆) δ ppm 8.37 (br d, J = 8.4 Hz, 2H), 7.97 (br d, J = 8.2 Hz, 2H), 7.27 (s, 2H), 7.17 (s, 2H), 7.07 (s, 2H), 3.80-3.62 (m, 1H), 3.24 (s, 3H), 3.13-2.98 (m, 1H), 2.85-2.73 (m, 1H), 1.96-1.80 (m, 2H) |
| 49 | 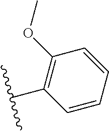 [SMILES: *c1ccccc1OC] | (R)-N-(1-(4-amino-7-(4- (methylsulfonyl)phenyl) pyrrolo[2,1- f][1,2,4]triazin-5- yl)piperidin-3-yl)-2- methoxybenzamide | 521.1 | A: 1.652 B: 1.469 | (500 MHz, DMSO-d₆) δ ppm 8.36 (d, J = 8.7 Hz, 2H), 8.30- 8.15 (m, 1H), 8.14-8.02 (m, 1H), 7.96 (d, J = 8.7 Hz, 2H), 7.90 (s, 1H), 7.76-7.67 (m, 1H), 7.52-7.42 (m, 1H), 7.24 (s, 1H), 7.14 (br d, J = 8.4 Hz, 1H), 7.03 (t, J = 7.6 Hz, 1H), 7.00-6.87 (m, 1H), 4.30-4.17 (m, 1H), 3.88 (s, 3H), 3.58-3.46 (m, 2H), 3.22 (s, 3H), 1.97-1,73 (m, 3H), 1.68-1.45 (m, 1H) |
| 50 | 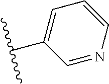 [SMILES: *c1cccnc1] | (R)-N-(1-(4-amino-7-(4- (methylsulfonyl)phenyl) pyrrolo [2,1- f][1,2,4]triazin-5- yl)piperidin-3- yl)nicotinamide, TFA | 492.1 | A: 1.402 B: 1.088 | (500 MHz, DMSO-d₆) δ ppm 8.61 (br d, J = 7.0 Hz, 1H), 8.36 (d, J = 8.5 Hz, 2H), 8.21 (br d, J = 7.9 Hz, 1H), 7.97 (br d, J = 8.5 Hz, 3H), 7.66-7.53 (m, 1H), 7.24 (s, 1H), 7.18- 7.03 (m, 1H), 4.23 (dt, J = 4.5, 2.2 Hz, 1H), 3.23 (s, 3H), 3.13-3.01 (m, 1H), 2.84-2.69 (m, 1H), 2.01-1.87 (m, 2H), 1.88-1.74 (m, 1H), 1.64-1.48 (m, 1H) |
| 51 | 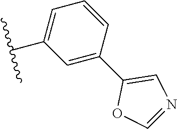 [SMILES: *c1cccc(-c2cnco2)c1] | (R)-N-(1-(4-amino-7-(4- (methylsulfonyl)phenyl) pyrrolo[2,1- f][1,2,4]triazin-5- yl)piperidin-3-yl)-3- (oxazol-5-yl)benzamide | 558.3 | A: 1.615 B: 1.367 | (500 MHz, DMSO-d₆) δ ppm 8.53 (br d, J = 7.9 Hz, 1H), 8.47 (s, 1H), 8.36 (br d, J = 8.5 Hz, 2H), 8.15 (s, 1H), 8.07- 7.97 (m, 1H), 7.95 (br d, J = 8.5 Hz, 2H), 7.90 (s, 1H), 7.87 (br d, J = 7.9 Hz, 1H), 7.83 (br d, J = 7.9 Hz, 1H), 7.57 (t. J = 7.8 Hz, 1H), 7.21 (s, 1H), 7.07-6.93 (m, 1H), 4.30- 4.11 (m, 1H), 3.22 (s, 3H), 3.12-3.01 (m, 1H), 2.81- 2.67 (m, 1H), 2.02-1.77 (m, 3H), 1.65-1.49 (m, 1H) |
| 52 | 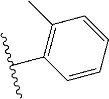 [SMILES: *c1ccccc1C] | (R)-N-(1-(4-amino-7-(4- (methylsulfonyl)phenyl) pyrrolo[2,1- f][1,2,4]triazin-5- yl)piperidin-3-yl)-2- methylbenzamide | 505.2 | A: 1.629 B: 1.389 | (500 MHz, DMSO-d₆) δ ppm 8.38 (br d, J = 8.9 Hz, 3H), 8.17-8.00 (m, 1H), 7.96 (br d, J = 8.9 Hz, 3H), 7.39-7.25 (m, 2H), 7.26-7.16 (m, 3H), 7.12-6.95 (m, 1H), 4.29- 4.11 (m, 1H), 3.23 (s, 3H), 2.87-2.60 (m, 1H), 2.30 (s, 3H), 2.01-1.83 (m, 2H), 1.84-1.68 (m, 1H), 1.58-1.40 (m, 1H) |
| 53 | 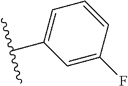 [SMILES: *c1cccc(F)c1] | (R)-N-(1-(4-amino-7-(4- (methylsulfonyl)phenyl) pyrrolo[2,1- f][1,2,4]triazin-5- yl)piperidin-3-yl)-3- fluorobenzamide | 509.0 | A: 1.663 B: 1.402 | (500 MHz, DMSO-d₆) δ ppm 8.47 (br d, J = 7.6 Hz, 1H), 8.42-8.33 (m, 2H), 8.16-8.02 (m, 1H), 7.96 (d, J = 8.5 Hz, 2H), 7.91 (s, 1H), 7.71 (br d, J = 8.2 Hz, 1H), 7.68- 7.62 (m, 1H), 7.52 (td, J = 8.0, 6.0 Hz, 1H), 7.41-7.34 (m, 1H), 7.23 (s, 1H), 7.02 (br dd, J = 3.7, 0.9 Hz, 1H), 4.27- 4.15 (m, 1H), 3.24 (s, 3H), 2.86-2.64 (m, 1H), 2.01- 1.77 (m, 3H), 1.63-1,48 (m, 1H) |
| 54 | 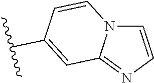 [SMILES: *c1ccn2ccnc2c1] | (R)-N-(1-(4-amino-7-(4- (methylsulfonyl)phenyl) pyrrolo[2,1- f][1,2,4]triazin-5- yl)piperidin-3- yl)imidazo[1,2- alpyridine-7- carboxamide | 531.2 | A: 1.273 B: 0.850 | (500 MHz, DMSO-d₆) δ ppm 8.71 (br d, J = 6.4 Hz, 2H), 8.37-8.31 (m, 2H), 8.26-8.15 (m, 2H), 7.96 (d, J = 8.5 Hz, 2H), 7.90 (s, 2H), 7.49 (br d, J = 7.0 Hz, 1H), 7.23 (s, 1H), 4.28-4.17 (m, 1H), 3.22 (s, 3H), 3.12-3.02 (m, 1H), 2.85-2.69 (m, 1H), 2.00-1.89 (m, 2H), 1.87-1.74 (m, 1H), 1.66-1.50 (m, 1H) |
| 55 | 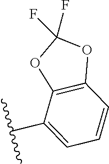 [SMILES: *c1cccc2c1OC(F)(F)O2] | (R)-N-(1-(4-amino-7-(4- (methylsulfonyl)phenyl) pyrrolo[2,1- f][1,2,4]triazin-5- yl)piperidin-3-yl)-2,2- difluorobenzo[d][1,3] dioxole-4-carboxamide | 571.3 | A: 1.788 B: 1.485 | (500 MHz, DMSO-d₆) δ ppm 8.45 (br d, J = 8.2 Hz, 1H), 8.39-8.32 (m, 2H), 8.08-7.99 (m, 1H), 7.96 (d, J = 8.9 Hz, 2H), 7.90 (s, 1H), 7.53 (dd, J = 7.9, 1.2 Hz, 1H), 7.47 (dd, J = 7.9, 0.9 Hz, 1H), 7.28 (t, J = 8.1 Hz, 1H), 7.23 (s, 1H), 7.08-6.94 (m, 1H), 4.27-4.14 (m, 1H), 3.89 (s, 3H), 3.16 (d, J = 5.2 Hz, 1H), 3,07-2.90 (m, 1H), 2.90- 2.69 (m, 1H), 1.96-1.86 (m, 2H), 1.84-1.75 (m, 1H), 1.62-1.46 (m, 2H) |
| 56 | 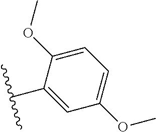 [SMILES: *c1cc(OC)ccc1OC] | (R)-N-(1-(4-amino-7-(4- (methylsulfonyl)phenyl) pyrrolo[2,1- f][1,2,4]triazin-5- yl)piperidin-3-yl)-2,5- dimethoxybenzamide, TFA | 551.2 | A: 1.646 B: 1.372 | (500 MHz, DMSO-d₆) δ ppm 8.33 (d, J = 8.5 Hz, 2H), 7.96 (d, J = 8.5 Hz, 2H), 7.32-7.27 (m, 1H), 7.24 (s, 1H), 7.11- 7.07 (m, 1H), 7.06-7.02 (m, 1H), 4.26-4.17 (m, 1H), 3.84 (s, 3H), 3.71 (s, 3H), 3.55 (br d, J = 4.3 Hz, 1H), 3.22 (s, 3H), 3.00-2.79 (m, 2H), 1.93-1.76 (m, 3H), 1.66- 1.48 (m, 1H) |
| 57 | 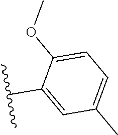 [SMILES: *c1cc(C)ccc1OC] | (R)-N-(1-(4-amino-7-(4- (methylsulfonyl)phenyl) pyrrolo[2,1- f][1,2,4]triazin-5- yl)piperidin-3-yl)-2- methoxy-5- methylbenzamide, TFA | 535.2 | A: 1.740 B: 1.437 | (500 MHz, DMSO-d₆) δ ppm 8.56-8.39 (m, 1H), 8.41- 8.32 (m, 1H), 8.29-8.15 (m, 1H), 8.04-7.92 (m, 2H), 7.53 (br dd, J = 3.5, 2.3 Hz, 1H), 7.27 (br d, J = 4.3 Hz, 2H), 7.03 (br dd, J = 8.5, 4.6 Hz, 1H), 4.31-4.17 (m, 1H), 3.85 (d, J = 4.3 Hz, 3H), 3.24 (d, J = 4.3 Hz, 3H), 3.02- 2.93 (m, 1H), 2.93-2.73 (m, 1H), 2.61-2.52 (m, 2H), 2.26 (br d, J = 4.3 Hz, 3H), 1.91-1.74 (m, 2H), 1.64- 1.49 (m, 1H), 1.31-1.20 (m, 1H) |
| 58 | 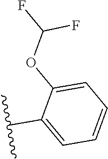 [SMILES: *c1ccccc1OC(F)F] | (R)-N-(1-(4-amino-7-(4- (methylsulfonyl)phenyl) pyrrolo[2,1- f][1,2,4]triazin-5- yl)piperidin-3-yl)-2- (difluoromethoxy) benzamide, TFA | 557.1 | A: 1.606 B: 1.345 | (500 MHz, DMSO-d₆) δ ppm 8.44-8.37 (m, 1H), 8.34 (d, J = 8.5 Hz, 2H), 8.00-7.95 (m, 2H), 7.93 (s, 1H), 7.50 (d, J = 7.3 Hz, 2H), 7.34-7.28 (m, 1H), 7.26-7.19 (m, 3H), 7.16-7.03 (m, 1H), 4.25-4.14 (m, 1H), 3.53-3.46 (m, 1H), 3.23 (s, 3H), 3.01-2.88 (m, 1H), 2.87-2.68 (m, 2H), 1.95-1.85 (m, 2H), 1.84-1.71 (m, 1H), 1.57- 1.41 (m, 1H) |
| 59 | 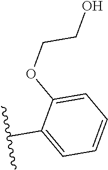 [SMILES: *c1ccccc1OCCO] | (R)-N-(1-(4-amino-7-(4- (methylsulfonyl)phenyl) pyrrolo[2,1- f][1,2,4]triazin-5- yl)piperidin-3-yl)-2-(2- hydroxyethoxy) benzamide | 551.3 | A: 1.502 B: 1.299 | (500 MHz, DMSO-d₆) δ ppm 8.57-8.42 (m, 1H), 8.35 (d, J = 8.5 Hz, 2H), 7.95 (d, J = 8.5 Hz, 2H), 7.89 (s, 1H), 7.84 (dd, J = 7.8, 1.7 Hz, 1H), 7.50-7.42 (m, 1H), 7.21 (s, 1H), 7.16 (d, J = 8.2 Hz, 1H), 7.05 (t, J = 7.5 Hz, 1H), 6.98 (br s, 1H), 4.18 (br t, J = 4.4 Hz, 2H), 3.83-3.74 (m, 2H), 3.22 (s, 3H), 3.10-2.96 (m, 1H), 2.87-2.69 (m, 1H), 2.01-1.86 (m, 2H), 1.85-1.74 (m, 1H), 1.59-1.44 (m, 1H) |
| 60 | 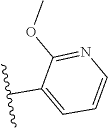 [SMILES: *c1cccnc1OC] | (R)-N-(1-(4-amino-7-(4- (methylsulfonyl)phenyl) pyrrolo[2,1- f][1,2,4]triazin-5- yl)piperidin-3-yl)-2- methoxynicotinamide | 522.3 | A: 1.543 B: 1.423 | (500 MHz, DMSO-d₆) δ ppm 8.35 (d, J = 8.5 Hz, 2H), 8.29 (dd, J = 4.9, 2.1 Hz, 2H), 8.12-8.05 (m, 1H), 7.96 (d, J = 8.9 Hz, 2H), 7.91 (s, 1H), 7.23 (s, 1H), 7.12 (dd, J = 7.3, 4.9 Hz, 1H), 4.26-4.17 (m, 1H), 3.96 (s, 3H), 3.22 (s, 3H), 3.01-2.78 (m, 2H), 1.95-1.83 (m, 2H), 1.83-1.70 (m, 1H), 1.67-1.49 (m, 1H) |
| 61 | 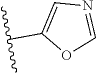 [SMILES: *c1cnco1] | (R)-N-(1-(4-amino-7-(4- (methylsulfonyl)phenyl) pyrrolo[2,1- f][1,2,4]triazin-5- yl)piperidin-3- yl)oxazole-5- carboxamide | 482.0 | A: 1.326 B: 1.120 | (500 MHz, DMSO-d₆) δ ppm 8.60 (br d, J = 7.9 Hz, 1H), 8.52 (s, 1H), 8.35 (d, J = 8.5 Hz, 2H), 7.96 (d, J = 8.9 Hz, 2H), 7.91 (s, 1H), 7.79 (s, 1H), 7.21 (s, 1H), 4.24-4.10 (m, 1H), 3.22 (s, 2H), 3.19-3.14 (m, 1H), 3.10-2.97 (m, 1H), 2.82-2.66 (m, 1H), 1.96-1.84 (m, 2H), 1.84- 1.73 (m, 1H), 1.63-1.46 (m, 1H) |
| 62 | 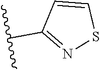 [SMILES: *c1ccsn1] | (R)-N-(1-(4-amino-7-(4- (methylsulfonyl)phenyl) pyrrolo[2,1- f][1,2,4]triazin-5- yl)piperidin-3- yl)isothiazole-3- carboxamide | 498.2 | A: 1.551 B: 1.304 | (500 MHz, DMSO-d₆) δ ppm 9.21-9.10 (m, 1H), 8.60 (br d, J = 8.7 Hz, 1H), 8.35 (br d, J = 8.8 Hz, 2H), 7.97 (br d. J = 8.4 Hz, 1H), 7.94 (s, 1H), 7.75 (d, J = 4.7 Hz, 1H), 7.23 (s, 1H), 4.30-4.14 (m, 1H), 3.23 (s, 3H), 3.11- 2.99 (m, 1H), 2.89-2.69 (m, 2H), 1.98-1.79 (m, 3H), 1.73-1.55 (m, 1H) |
| 63 | 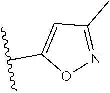 [SMILES: *c1cc(C)no1] | (R)-N-(1-(4-amino-7-(4- (methylsulfonyl)phenyl) pyrrolo[2,1- f][1,2,4]triazin-5- yl)piperidin-3-yl)-3- methylisoxazole-5- carboxamide, TFA | 496.3 | A: 1.383 B: 1.105 | (500 MHz, DMSO-d₆) δ ppm 8.88 (d, J = 7.6 Hz, 1H), 8.36 (d, J = 8.5 Hz, 2H), 7.98 (d, J = 8.9 Hz, 2H), 7.95 (s, 1H), 7.24 (s, 1H), 6.94 (s, 1H), 4.28-4.12 (m, 1H), 3.56- 3.37 (m, 1H), 3.24 (s, 3H), 3.10-3.02 (m, 1H), 2.82- 2.70 (m, 1H), 2.30 (s, 3H), 1.97-1.86 (m, 2H), 1.86- 1.74 (m, 1H) |
| 64 | 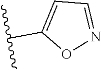 [SMILES: *c1ccno1] | (R)-N-(1-(4-amino-7-(4- (methylsulfonyl)phenyl) pyrrolo[2,1- f][1,2,4]triazin-5- yl)piperidin-3- yl)isoxazole-5- carboxamide | 482.1 | A: 1.314 B: 1.227 | (500 MHz, DMSO-d₆) δ ppm 8.94 (d, J = 7.9 Hz, 1H), 8,70 (d. J = 1.8 Hz, 1H), 8.34 (d, J = 8.5 Hz, 2H), 7.95 (d, J = 8.9 Hz, 2H), 7.19 (s, 1H), 7.06 (d, J = 1.8 Hz, 1H), 7.03-6.92 (m, 1H), 4.24-4.12 (m, 1H), 3.21 (s, 3H), 3.03 (ddd, J = 5.8, 4.1, 2.0 Hz, 1H), 2.83-2.66 (m, 1H), 1.97-1.85 (m, 2H), 1.84-1.73 (m, 1H), 1.62-1.47 (m, 1H) |
| 65 | 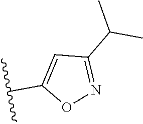 [SMILES: *c1cc(C(C)C)no1] | (R)-N-(1-(4-amino-7-(4- (methylsulfonyl)phenyl) pyrrolo [2,1- f][1,2,4]triazin-5- yl)piperidin-3-yl)-3- isopropylisoxazole-5- carboxamide | 524.3 | A: 1.708 B: 1.461 | (500 MHz, DMSO-d₆) δ ppm 8.85 (d, J = 7.3 Hz, 1H), 8.35 (d, J = 8.9 Hz, 2H), 7.95 (d, J = 8.5 Hz, 2H), 7.89 (s, 1H), 7.20 (s, 1H), 7.04 (s, 1H), 4.25-4.11 (m, 1H), 3.22 (s, 3H), 3.09-2.99 (m, 1H), 2.83-2.64 (m, 2H), 1.96-1.84 (m, 2H), 1.84-1.72 (m, 1H), 1.63-1.46 (m, 1H), 1.23 (d, J = 7.0 Hz, 6H) |
| 66 | 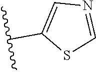 [SMILES: *c1cncs1] | (R)-N-(1-(4-amino-7-(4- (methylsulfonyl)phenyl) pyrrolo[2,1- f][1,2,4]triazin-5- yl)piperidin-3- yl)thiazole-5- carboxamide | 498.2 | A: 1.394 B: 1.174 | (500 MHz, DMSO-d₆) δ ppm 9.20 (s, 1H), 8.64 (br d, J = 7.3 Hz, 1H), 8.50 (s, 1H), 8.39-8.32 (m, 2H), 7.99- 7.94 (m, 2H), 7.91 (s, 1H), 7.22 (s, 1H), 4.27-4.09 (m, 2H), 3.22 (s, 3H), 3.11-2.97 (m, 1H), 2.80-2.68 (m, 1H), 2.01-1.86 (m, 2H), 1.85-1.75 (m, 1H), 1.64-1.42 (m, 2H) |
| 67 | 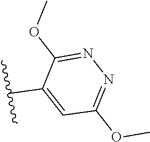 [SMILES: *c1cc(OC)nnc1OC] | (R)-N-(1-(4-amino-7-(4- (methylsulfonyl)phenyl) pyrrolo[2,1- f][1,2,4]triazin-5- yl)piperidin-3-yl)-3,6- dimethoxypyridazine-4- carboxamide | 553.3 | A: 1.566 B: 1.324 | (500 MHz, DMSO-d₆) δ ppm 8.65-8.55 (m, 1H), 8.35 (br d, J = 8.6 Hz, 2H), 8.31-8.19 (m, 1H), 7.97 (br d, J = 8.1 Hz, 2H), 7.94 (s, 1H), 7.25 (d. J = 8.2 Hz, 2H), 4.24- 4.13 (m, 1H), 3.98 (s, 3H), 3.96 (s, 3H), 3.51-3.39 (m, 2H), 3.24 (s, 3H), 3.09-2.77 (m, 1H), 1.96-1.83 (m, 2H), 1.83-1.71 (m, 1H), 1.63-1.44 (m, 1H) |
| 68 | 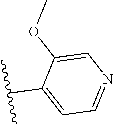 [SMILES: *c1ccncc1OC] | (R)-N-(1-(4-amino-7-(4- (methylsulfonyl)phenyl) pyrrolo[2,1- f][1,2,4]triazin-5- yl)piperidin-3-yl)-3- methoxyisonicotinamide | 522.3 | A: 1.357 B: 0.949 | (500 MHz, DMSO-d₆) δ ppm 8.51 (s, 1H), 8.45-8.37 (m, 1H), 8.34 (d, J = 8.9 Hz, 2H), 8.30 (br s, 1H), 7.96 (d, J = 8.5 Hz, 2H), 7.92 (s, 1H), 7.50 (br d, J = 4.6 Hz, 1H), 7.26 (s, 1H), 7.23 (s, 1H), 7.16 (s, 1H), 7.06 (s, 1H), 4.26- 4.16 (m, 1H), 3.96 (s, 3H), 3.27-3.23 (m, 1H), 3.22 (s, 3H), 3.02-2.78 (m, 2H), 1.93-1.83 (m, 2H), 1.84-1.72 (m, 1H), 1.63-1.47 (m, 1H) |
| 69 | 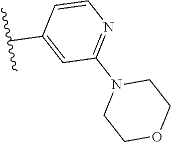 [SMILES: *c1ccnc(N2CCOCC2)c1] | (R)-N-(1-(4-amino-7-(4- (methylsulfonyl)phenyl) pyrrolo[2,1- f][1,2,4]triazin-5- yl)piperidin-3-yl)-2- morpholinoisonicotinamide | 577.3 | A: 1.424 B: 0.938 | (500 MHz, DMSO-d₆) δ ppm 8.50 (br d, J = 7.6 Hz, 1H), 8.37 (d, J = 8.5 Hz, 2H), 8.21 (d. J = 5.2 Hz, 1H), 8.14- 8.02 (m, 1H), 7.96 (d, J = 8.5 Hz, 2H), 7.91 (s, 1H), 7.22 (s, 1H), 7.13 (s, 1H), 7.02 (d, J = 5.2 Hz, 1H), 4.26-4.12 (m, 1H), 3.75-3.66 (m, 4H), 3.48 (br t, J = 4,9 Hz, 2H), 3.11-2.98 (m, 1H), 2.83-2.66 (m, 1H), 2.03-1.75 (m, 3H), 1.60-1.45 (m, 1H) |
| 70 | 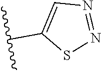 [SMILES: *c1cnns1] | (R)-N-(1-(4-amino-7-(4- (methylsulfonyl)phenyl) pyrrolo[2,1- f][1,2,4]triazin-5- yl)piperidin-3-yl)-1,2,3- thiadiazole-5- carboxamide | 499.1 | A: 1.537 B: 1.201 | (500 MHz, DMSO-d₆) δ ppm 9.31 (s, 1H), 9.05 (br d, J = 7.6 Hz, 1H), 8.30 (d, J = 8.9 Hz, 2H), 7.91 (d, J = 8.5 Hz, 2H), 7.85 (s, 1H), 7.17 (s, 1H), 7.06-6.80 (m, 1H), 4.20- 4.07 (m, 1H), 3.51 (br dd, J = 3.4, 2.4 Hz, 1H), 3.17 (s, 3H), 3.06-2.95 (m, 1H), 2.80-2.65 (m, 1H), 1.98-1.82 (m, 2H), 1.82-1.69 (m, 1H), 1.59-1.43 (m, 1H) |
| 71 | 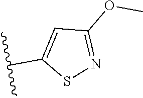 [SMILES: *c1cc(OC)ns1] | (R)-N-(1-(4-amino-7-(4- (methylsulfonyl)phenyl) pyrrolo[2,1- f][1,2,4]triazin-5- yl)piperidin-3-yl)-3- methoxyisothiazole-5- carboxamide | 528.1 | A: 1.630 B: 1.433 | (500 MHz, DMSO-d₆) δ ppm 8.80-8.72 (m, 1H), 8.40- 8.33 (m, 2H), 8.14-7.99 (m, 1H), 7.96 (d, J = 8.6 Hz, 2H), 7.90 (s, 1H), 7.38 (br s, 1H), 7.22 (d, J = 1.2 Hz, 1H), 7.07-6.89 (m, 1H), 4.28-4.09 (m, 1H), 3.94 (s, 3H), 3.23 (d. J = 0.7 Hz, 3H), 3.10-2.98 (m, 1H), 2.85-2.64 (m, 1H), 2.05-1.73 (m, 3H), 1.61-1.44 (m, 1H) |
| 72 | 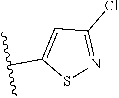 [SMILES: *c1cc(Cl)ns1] | (R)-N-(1-(4-amino-7-(4- (methylsulfonyl)phenyl) pyrrolo[2,1- f][1,2,4]triazin-5- yl)piperidin-3-yl)-3- chloroisothiazole-5- carboxamide, TFA | 532.0 | A: 1.702 B: 1.424 | (500 MHz, DMSO-d₆) δ ppm 8.90 (d, J = 7.6 Hz, 1H), 8.34 (d, J = 8.5 Hz, 2H), 8.00-7.94 (m, 3H), 7.92 (s, 1H), 7.23 (s, 1H), 4.25-4.12 (m, 1H), 3.52-3.40 (m, 1H), 3.23 (s, 3H), 3.11-3.00 (m, 1H), 2.84-2.72 (m, 1H), 2.00-1.87 (m, 2H), 1.87-1.76 (m, 1H), 1.61-1.46 (m, 1H) |
| 73 | 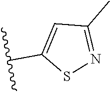 [SMILES: *c1cc(C)ns1] | (R)-N-(1-(4-amino-7-(4- (methylsulfonyl)phenyl) pyrrolo[2,1- f][1,2,4]triazin-5- yl)piperidin-3-yl)-3- methylisothiazole-5- carboxamide | 512.2 | A: 1.617 B: 1.273 | (500 MHz, DMSO-d₆) δ ppm 8.73 (br d, J = 7.6 Hz, 1H), 8.35 (d, J = 8.5 Hz, 2H), 7.95 (d, J = 8.5 Hz, 2H), 7.89 (s, 1H), 7.71 (s, 1H), 7.20 (s, 1H), 7.10-6.88 (m, 1H), 4.21- 4.07 (m, 1H), 3.50 (br d, J = 2.1 Hz, 1H), 3.21 (s, 3H), 3.12-3.00 (m, 1H), 2.84-2.71 (m, 1H), 2.45 (s, 3H), 1.99-1.86 (m, 2H), 1.84-1.72 (m, 1H), 1.62-1.44 (m, 1H) |
| 74 | 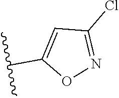 [SMILES: *c1cc(Cl)no1] | (R)-N-(1-(4-amino-7-(4- (methylsulfonyl)phenyl) pyrrolo[2,1- f][1,2,4]triazin-5- yl)piperidin-3-yl)-3- chloroisoxazole-5- carboxamide, TFA | 516.1 | A: 1.647 B: 1.377 | (500 MHz, DMSO-d₆) δ ppm 9.06 (br d, J = 7.6 Hz, 1H), 8.32 (d, J = 8.5 Hz, 2H), 7.96 (d, J = 8.5 Hz, 2H), 7.92 (s, 1H), 7.34 (s, 1H), 7.21 (s, 1H), 4.24-4.12 (m, 1H), 3.65- 3.49 (m, 1H), 3.21 (s, 3H), 3.09-2.99 (m, 1H), 2.85- 2.70 (m, 1H), 1.98-1.85 (m, 2H), 1.86-1.74 (m, 1H), 1.62-1.47 (m, 1H) |
| 75 | 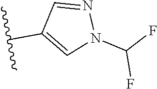 [SMILES: *c1cnn(C(F)F)c1] | (R)-N-(1-(4-amino-7-(4- (methylsulfonyl)phenyl) pyrrolo[2,1- f][1,2,4]triazin-5- yl)piperidin-3-yl)-1- (difluoromethyl)-1H- pyrazole-4- carboxamide, TFA | 531.1 | A: 1.503 B: 1.279 | (500 MHz, DMSO-d₆) δ ppm 8.69 (s, 1H), 8.34 (d, J = 8.9 Hz, 2H), 8.26 (br d, J = 7.6 Hz, 1H), 8.17 (s, 1H), 8.00- 7.93 (m, 3H), 7.95-7.68 (m, 1H), 7.27-7.20 (m, 1H), 4.28-4.08 (m, 1H), 3.23 (s, 3H), 3.11-3.03 (m, 1H), 2.83-2.68 (m, 1H), 1.97-1.77 (m, 3H), 1.57-1.43 (m, 1H) |
| 76 | 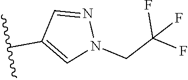 [SMILES: *c1cnn(CC(F)(F)F)c1] | (R)-N-(1-(4-amino-7-(4- (methylsulfonyl)phenyl) pyrrolo[2,1- f][1,2,4]triazin-5- yl)piperidin-3-yl)-1- (2,2,2-trifluoroethyl)- 1H-pyrazole-4- carboxamide | 563.1 | A: 1.544 B: 1.327 | (500 MHz, DMSO-d₆) δ ppm 8.35 (d, J = 8.5 Hz, 2H), 8.31 (s, 1H), 8.14 (br d, J = 7.9 Hz, 1H), 8.00 (s, 1H), 7.95 (d, J = 8.9 Hz, 2H), 7.89 (s, 1H), 7.20 (s, 1H), 7.10-6.89 (m, 1H), 5.16 (q, J = 9.0 Hz, 2H), 4.21-4.07 (m, 1H), 3.22 (s, 3H), 3.09-3.02 (m, 1H), 2.78-2.61 (m, 1H), 1.98-1.84 (m, 2H), 1.84-1.68 (m, 1H), 1.60-1.38 (m, 1H) |
| 77 | 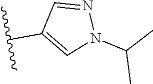 [SMILES: *c1cnn(C(C)C)c1] | (R)-N-(1-(4-amino-7-(4- (methylsulfonyl)phenyl) pyrrolo[2,1- f][1,2,4]triazin-5- yl)piperidin-3-yl)-1- isopropyl-1H-pyrazole- 4-carboxamide, TFA | 523.2 | A: 1.509 B: 1.292 | (500 MHz, DMSO-d₆) δ ppm 8.33 (d, J = 8.5 Hz, 2H), 8.20 (s, 1H), 7.96 (br d, J = 8.5 Hz, 3H), 7.92 (s, 1H), 7.86 (s, 1H), 7.21 (s, 1H), 4.48 (dquin, J = 13.3, 6.4 Hz, 1H), 4.20- 4.05 (m, 1H), 3.56-3.47 (m, 1H), 3.22 (s, 3H), 3.11- 3.02 (m, 1H), 2.79-2.61 (m, 1H), 1.96-1.73 (m, 3H), 1.54-1.43 (m, 1H), 1.40 (d. J = 6.7 Hz, 6H) |
| 78 | 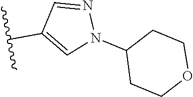 [SMILES: *c1cnn(C2CCOCC2)c1] | (R)-N-(1-(4-amino-7-(4- (methylsulfonyl)phenyl) pyrrolo[2,1- f][1,2,4]triazin-5- yl)piperidin-3-yl)-1- (tetrahydro-2H-pyran-4- yl)-1H-pyrazole-4- carboxamide | 565.1 | A: 1.432 B: 1.231 | (500 MHz, DMSO-d₆) δ ppm 8.36 (d, J = 8.5 Hz, 2H), 8.25 (s, 1H), 8.00-7.91 (m, 4H), 7.89 (s, 1H), 7.22 (s, 1H), 4.45-4.36 (m, 1H), 4.20-4.09 (m, 1H), 4.00-3.89 (m, 2H), 3.23 (s, 3H), 3.05 (br s, 1H), 2.87-2.61 (m, 1H), 2.05-1.95 (m, 2H), 1.96-1.74 (m, 5H), 1.58-1.41 (m, 1H) |
| 79 | 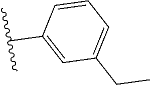 [SMILES: *c1cccc(CC)c1] | (R)-N-(1-(4-amino-7-(4- (methylsulfonyl)phenyl) pyrrolo[2,1- f][1,2,4]triazin-5- yl)piperidin-3-yl)-3- ethylbenzamide, TFA | 519.2 | A: 1.858 B: 1.597 | (500 MHz, DMSO-d₆) δ ppm 8.39-8.29 (m, 3H), 7.96 (d, J = 8.5 Hz, 2H), 7.93 (s, 1H), 7.67 (s, 1H), 7.66-7.58 (m, 1H), 7.35 (d, J = 4.6 Hz, 2H), 7.23 (s, 1H), 4.30-4.13 (m, 1H), 3.51-3.32 (m, 1H), 3.23 (s, 3H), 3.12-3.02 (m, 1H), 2.81-2.71 (m, 1H), 2.64 (q, J = 7.5 Hz, 2H), 1.99-1.75 (m, 3H), 1.62-1.48 (m, 1H), 1.19 (t, J = 7.5 Hz, 3H) |
| 80 | 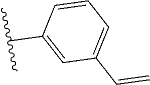 [SMILES: *c1cccc(C=C)c1] | (R)-N-(1-(4-amino-7-(4- (methylsulfonyl)phenyl) pyrrolo[2,1- f][1,2,4]triazin-5- yl)piperidin-3-yl)-3- vinylbenzamide | 517.1 | A: 1.817 B: 1.598 | (500 MHz, DMSO-d₆) δ ppm 8.41 (br d, J = 7.6 Hz, 1H), 8.37 (d, J = 8.5 Hz, 2H), 8.13-8.00 (m, 1H), 7.96 (d, J = 8.5 Hz, 2H), 7.91 (s, 2H), 7.73 (br d, J = 7.3 Hz, 1H), 7.62 (br d, J = 7.6 Hz, 1H), 7.47-7.39 (m, 1H), 7.22 (s, 1H), 7.12-6.93 (m, 1H), 6.79 (dd, J = 17.5, 10.8 Hz, 1H), 5.91 (d, J = 17.7 Hz, 1H), 5.33 (d, J = 11.0 Hz, 1H), 4.28- 4.06 (m, 1H), 3.23 (s, 3H), 3.13-3.01 (m, 1H), 2.83- 2.67 (m, 1H), 2.00-1.76 (m, 3H), 1.62-1.47 (m, 1H) |
| 81 | 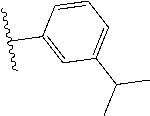 [SMILES: *c1cccc(C(C)C)c1] | (R)-N-(1-(4-amino-7-(4- (methylsulfonyl)phenyl) pyrrolo[2,1- f][1,2,4]triazin-5- yl)piperidin-3-yl)-3- isopropylbenzamide | 532.9 | A: 1.923 B: 1.656 | (500 MHz, DMSO-d₆) δ ppm 8.35 (br d, J = 8.5 Hz, 3H), 7.95 (d, J = 8.9 Hz, 2H), 7.94-7.88 (m, 1H), 7.68 (s, 1H), 7.63 (br d, J = 7.6 Hz, 1H), 7.43-7.30 (m, 2H), 7.20 (s, 1H), 7.10-6.91 (m, 1H), 4.24-4.12 (m, 1H), 3.21 (s, 3H), 3.11-3.02 (m, 1H), 2.96-2.86 (m, 1H), 2.83-2.66 (m, 2H), 2.00-1.74 (m, 3H), 1.63-1.48 (m, 1H), 1.20 (d, J = 7.0 Hz, 6H) |
| 82 | 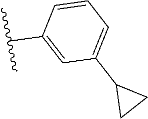 [SMILES: *c1cccc(C2CC2)c1] | (R)-N-(1-(4-amino-7-(4- (methylsulfonyl)phenyl) pyrrolo[2,1- f][1,2,4]triazin-5- yl)piperidin-3-yl)-3- cyclopropylbenzamide | 531.2 | A: 1.865 B: 1.604 | (500 MHz, DMSO-d₆) δ ppm 8.35 (d, J = 8.9 Hz, 2H), 8.06- 7.98 (m, 1H), 7.95 (d, J = 8.9 Hz, 2H), 7.89 (s, 1H), 7.57 (br d, J = 7.3 Hz, 1H), 7.49 (s, 1H), 7.31 (t, J = 7.6 Hz, 1H). 7.24-7.16 (m, 2H), 7.09-6.90 (m, 1H), 4.24-4.11 (m, 1H), 3.60-3.47 (m, 1H), 3.21 (s, 3H), 3.09-3.01 (m, 1H), 2.80-2.65 (m, 1H), 2.01-1.74 (m, 4H), 1.62-1.44 (m, 1H), 1.02-0.92 (m, 2H), 0.75-0.65 (m, 2H) |
| 83 | 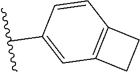 [SMILES: *c1ccc2c(c1)CC2] | (R)-N-(1-(4-amino-7-(4- (methylsulfonyl)phenyl) pyrrolo[2,1- f][1,2,4]triazin-5- yl)piperidin-3- yl)bicyclo[4.2.0]octa- 1(6),2,4-triene-3- carboxamide, TFA | 516.9 | A: 1.763 B: 1.498 | (500 MHz, DMSO-d₆) δ ppm 8.36 (d, J = 8.5 Hz, 2H), 8.28 (br d, J = 7.6 Hz, 1H), 7.98 (d, J = 8.5 Hz, 2H), 7.93 (s, 1H), 7.70 (br d, J = 7.6 Hz, 1H), 7.54 (s, 1H), 7.23 (s, 1H), 7.16 (d, J = 7.3 Hz, 1H), 4.26-4.11 (m, 1H), 3.24 (s, 3H), 3.17 (s, 4H), 3.11-2.98 (m, 1H), 2.82-2.69 (m, 1H), 2.00-1.87 (m, 2H), 1.86-1.73 (m, 1H), 1.65-1.46 (m, 1H) |
| 84 | 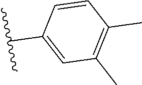 [SMILES: *c1ccc(C)c(C)c1] | (R)-N-(1-(4-amino-7-(4- (methylsulfonyl)phenyl) pyrrolo[2,1- f][1,2,4]triazin-5- yl)piperidin-3-yl)-3,4- dimethylbenzamide, TFA | 519.2 | A: 1.901 B: 1.622 | (500 MHz, DMSO-d₆) δ ppm 8.35 (br d, J = 8.8 Hz, 2H), 8.25 (br d, J = 7.9 Hz, 1H), 7.96 (d, J = 8.6 Hz, 2H), 7.92 (s, 1H), 7.62 (s, 1H), 7.56 (br d, J = 8.3 Hz, 1H), 7.27- 7.17 (m, 2H), 4.26-4.12 (m, 1H), 3.63-3.41 (m, 3H), 3.23 (s, 3H), 3.11-3.00 (m, 1H), 2.80-2.65 (m, 1H), 2.25 (s, 6H), 1.97-1.74 (m, 3H), 1.61-1.46 (m, 1H) |
| 85 | 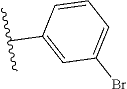 [SMILES: *c1cccc(Br)c1] | (R)-N-(1-(4-amino-7-(4- (methylsulfonyl)phenyl) pyrrolo[2,1- f][1,2,4]triazin-5- yl)piperidin-3-yl)-3- bromobenzamide, TFA | 569.0 | A: 1.831 B: 1.563 | (500 MHz, DMSO-d₆) δ ppm 8.52 (br d, J = 7.9 Hz, 1H), 8.34 (d, J = 8.5 Hz, 2H), 8.02 (d. J = 1.8 Hz, 1H), 7.99- 7.92 (m, 3H), 7.84 (d, J = 7.9 Hz, 1H), 7.72 (br d, J = 7.0 Hz, 1H), 7.43 (t, J = 7.9 Hz, 1H), 7.23 (s, 1H), 4.27-4.12 (m, 1H), 3.23 (s, 3H), 3.11-3.02 (m, 1H), 2.82-2.69 (m, 1H), 1.98-1.79 (m, 3H), 1.60-1.48 (m, 1H) |
| 86 | 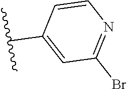 [SMILES: *c1ccnc(Br)c1] | (R)-N-(1-(4-amino-7-(4- (methylsulfonyl)phenyl) pyrrolo[2,1- f][1,2,4]triazin-5- yl)piperidin-3-yl)-2- bromoisonicotinamide, TFA | 570.0 | A: 1.638 B: 1.407 | (500 MHz, DMSO-d₆) δ ppm 8.78 (br d, J = 7.8 Hz, 1H), 8.52 (d, J = 5.1 Hz, 1H), 8.38-8.30 (m, 2H), 8.03-7.92 (m, 4H), 7.77 (br d, J = 5.1 Hz, 1H), 7.29-7.21 (m, 1H), 4.28-4.14 (m, 1H), 3.23 (s, 3H), 3.09-2.99 (m, 1H), 2.86-2.67 (m, 1H), 1.98-1.74 (m, 3H), 1.62-1.47 (m, 1H) |
| 87 | 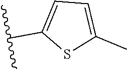 [SMILES: *c1ccc(C)s1] | (R)-N-(1-(4-amino-7-(4- (methylsulfonyl)phenyl) pyrrolo[2,1- f][1,2,4]triazin-5- yl)piperidin-3-yl)-5- methylthiophene-2- carboxamide | 511.3 | A: 1.717 B: 1.490 | (500 MHz, DMSO-d₆) δ ppm 8.37 (br d, J = 8.5 Hz, 2H), 8.25 (br d, J = 7.2 Hz, 1H), 7.96 (br d, J = 8.7 Hz, 3H), 7.62 (br s, 1H), 7.22 (s, 1H), 7.08-6.90 (m, 1H), 6.83 (d, J = 3.4 Hz, 1H), 4.20-4.06 (m, 1H), 3.48-3.35 (m, 1H), 3.23 (s, 3H), 3.10-3.01 (m, 1H), 2.79-2.60 (m, 1H), 2.45 (s, 3H), 2.00-1.75 (m, 3H), 1.58-1.45 (m, 1H) |
| 88 | 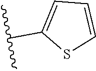 [SMILES: *c1cccs1] | (R)-N-(1-(4-amino-7-(4- (methylsulfonyl)phenyl) pyrrolo[2,1- f][1,2,4]triazin-5- yl)piperidin-3- yl)thiophene-2- carboxamide, TFA | 497.0 | A: 1.609 B: 1.396 | (500 MHz, DMSO-d₆) δ ppm 8.38 (br d, J = 8.2 Hz, 1H), 8.33 (d, J = 8.5 Hz, 2H), 8.00-7.95 (m, 3H), 7.84-7.80 (m, 1H), 7.74 (d, J = 4.3 Hz, 1H), 7.25 (s, 1H), 7.14 (dd, J = 5.0, 3.8 Hz, 1H), 4.27-4.12 (m, 1H), 3.24 (s, 3H), 3.13-3.05 (m, 1H), 2.81-2.68 (m, 2H), 1.99-1.91 (m, 1H), 1.91-1.80 (m, 2H), 1.63-1.46 (m, 1H) |
| 89 | 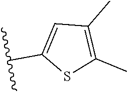 [SMILES: *c1cc(C)c(C)s1] | (R)-N-(1-(4-amino-7-(4- (methylsulfonyl)phenyl) pyrrolo[2,1- f][1,2,4]triazin-5- yl)piperidin-3-yl)-4,5- dimethylthiophene-2- carboxamide | 525.1 | A: 1.778 B: 1.590 | (500 MHz, DMSO-d₆) δ ppm 8.41-8.32 (m, 2H), 8.19 (br d, J = 7.4 Hz, 1H), 8.12-7.98 (m, 1H), 7.95 (d, J = 8.5 Hz, 2H), 7.90 (s, 1H), 7.51 (s, 1H), 7.20 (s, 1H), 7.07- 6.88 (m, 1H), 4.20-4.00 (m, 1H), 3.27-3.15 (m, 3H), 2.81-2.58 (m, 2H), 2.30 (s, 3H), 2.09 (s, 3H), 1,95- 1.84 (m, 2H), 1.85-1.71 (m, 1H), 1.56-1.39 (m, 1H) |
| 90 | 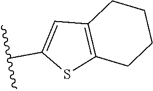 [SMILES: *c1cc2c(s1)CCCC2] | (R)-N-(1-(4-amino-7-(4- (methylsulfonyl)phenyl) pyrrolo[2,1- f][1,2,4]triazin-5- yl)piperidin-3-yl)- 4,5,6,7- tetrahydrobenzo[b] thiophene-2-carboxamide | 551.2 | A: 1.926 B: 1.639 | (500 MHz, DMSO-d₆) δ ppm 8.35 (d, J = 8.5 Hz, 2H), 8.20 (br d, J = 7.6 Hz, 1H), 8.08-7.98 (m, 1H), 7.95 (d, J = 8.9 Hz, 2H), 7.89 (s, 1H), 7.48 (s, 1H), 7.20 (s, 1H), 7.06- 6.88 (m, 1H), 4.18-4.03 (m, 1H), 3.22 (s, 3H), 3.09- 3.01 (m, 1H), 2.70 (brt, J = 5.5 Hz, 3H), 1.94-1.85 (m, 2H), 1.83-1.63 (m, 6H), 1.59-1.43 (m, 1H) |
| 91 | 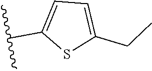 [SMILES: *c1ccc(CC)s1] | (R)-N-(1-(4-amino-7-(4- (methylsulfonyl)phenyl) pyrrolo[2,1- f][1,2,4]triazin-5- yl)piperidin-3-yl)-5- ethylthiophone-2- carboxamide | 525.1 | A: 1.748 B: 1.648 | (500 MHz, DMSO-d₆) δ ppm 8.34 (br d, J = 8.5 Hz, 2H), 8.27 (br d, J = 8.2 Hz, 1H), 7.95 (br d, J = 8.9 Hz, 2H), 7.88 (s, 1H), 7.61 (br d, J = 3.4 Hz, 1H), 7.19 (s, 1H), 7.08- 6.90 (m, 1H), 6.85 (d, J = 3,7 Hz, 1H), 4.19-4.03 (m, 1H), 3.21 (s, 3H), 3.09-3.00 (m, 1H), 2.79 (q, J = 7.9 Hz, 2H), 2.75-2.64 (m, 1H), 1.97-1.83 (m, 2H), 1.83-1.70 (m, 1H), 1.60-1.41 (m, 1H), 1.21 (t, J = 7.5 Hz, 3H) |
| 92 | 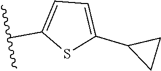 [SMILES: *c1ccc(C2CC2)s1] | (R)-N-(1-(4-amino-7-(4- (methylsulfonyl)phenyl) pyrrolo[2,1- f][1,2,4]triazin-5- yl)piperidin-3-yl)-5- cyclopropylthiophene-2- carboxamide, TFA | 537.4 | A: 1.887 B: 1.578 | (500 MHz, DMSO-d₆) δ ppm 8.30 (d, J = 8.7 Hz, 2H), 8.19 (br d, J = 7.7 Hz, 1H), 7.96-7.89 (m, 3H), 7.56 (d, J = 3.8 Hz, 1H), 7.19 (s, 1H), 6.78 (d, J = 3.8 Hz, 1H), 4.18-4.02 (m, 1H), 3.20 (s, 3H), 3.07-2.97 (m, 1H), 2.74-2.61 (m, 2H), 2.15-2.06 (m, 1H), 1.91-1.73 (m, 3H), 1.55- 1.42 (m, 1H), 1.02-0.96 (m, 2H), 0.69-0.61 (m, 2H) |
| 93 | 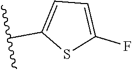 [SMILES: *c1ccc(F)s1] | (R)-N-(1-(4-amino-7-(4- (methylsulfonyl)phenyl) pyrrolo[2,1- f][1,2,4]triazin-5- yl)piperidin-3-yl)-5- fluorothiophene-2- carboxamide | 515.1 | A: 1.728 B: 1.454 | (500 MHz, DMSO-d₆) δ ppm 8.43 (br d, J = 7.6 Hz, 1H), 8.36 (br d, J = 8.2 Hz, 2H), 8.12-8.00 (m, 1H), 7.96 (br d, J = 8.5 Hz, 2H), 7.90 (s, 1H), 7.59 (br t, J = 3.8 Hz, 1H), 7.21 (s, 1H), 7.08-6.88 (m, 1H), 6.79 (br d, J = 2.1 Hz, 1H), 4.21-4.05 (m, 1H), 3.23 (s, 2H), 3.11-3.01 (m, 1H), 2.79-2.63 (m, 1H), 1.99-1.75 (m, 3H), 1.61-1.44 (m, 1H) |
| 94 | 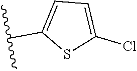 [SMILES: *c1ccc(Cl)s1] | (R)-N-(1-(4-amino-7-(4- (methylsulfonyl)phenyl) pyrrolo[2,1- f][1,2,4]triazin-5- yl)piperidin-3-yl)-5- chlorothiophene-2- carboxamide | 530.8 | A: 1.838 B: 1.553 | (500 MHz, DMSO-d₆) δ ppm 8.49 (d, J = 8.0 Hz, 1H), 8.41- 8.30 (m, 2H), 8.10-7.98 (m, 1H), 7.98-7.92 (m, 1H), 7.90 (s, 1H), 7.70 (d. J = 4.0 Hz, 1H), 7.21 (s, 1H), 7.16 (d, J = 4.1 Hz, 1H), 4.21-4.02 (m, 1H), 3.22 (s, 3H), 3.12- 3.00 (m, 1H), 2.79-2.66 (m, 1H), 1.97-1.74 (m, 3H), 1.60-1.44 (m, 1H) |
| 93 | 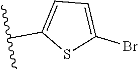 [SMILES: *c1ccc(Br)s1] | (R)-N-(1-(4-amino-7-(4- (methylsulfonyl)phenyl) pyrrolo[2,1- f][1,2,4]triazin-5- yl)piperidin-3-yl)-5- bromothiophene-2- carboxamide, TFA | 574.9 | A: 1.854 B: 1.553 | (500 MHz, DMSO-d₆) δ ppm 8.47 (br d, J = 7.6 Hz, 1H), 8.35 (br d, J = 8.5 Hz, 2H), 7.97 (br d, J = 8.5 Hz, 2H), 7.94 (s, 1H), 7.66 (d, J = 4.3 Hz, 1H), 7.27 (d, J = 4,0 Hz, 1H), 7.25-7.20 (m, 1H), 4.21-4.08 (m, 1H), 3.23 (s, 3H), 3.13-3.02 (m, 1H), 2.80-2.65 (m, 2H), 1.99-1.76 (m, 3H), 1.63-1.45 (m, 1H) |
| 96 | 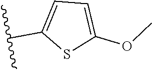 [SMILES: *c1ccc(OC)s1] | (R)-N-(1-(4-amino-7-(4- (methylsulfonyl)phenyl) pyrrolo[2,1- f][1,2,4]triazin-5- yl)piperidin-3-yl)-5- methoxythiophene-2- carboxamide | 527.1 | A: 1.693 B: 1.426 | (500 MHz, DMSO-d₆) δ ppm 8.35 (d, J = 8.6 Hz, 2H), 8.18 (d, J = 7.8 Hz, 1H), 7.99-7.93 (m, 2H), 7.89 (s, 1H), 7.53 (d, J = 4.1 Hz, 1H), 7.20 (s, 1H), 6.33 (d, J = 4.2 Hz, 1H), 4.16-4.03 (m, 1H), 3.87 (s, 3H), 3.22 (s, 3H), 3,10- 2.96 (m, 1H), 2.78-2.59 (m, 2H), 2.03-1.83 (m, 2H), 1.84-1.71 (m, 1H), 1.59-1.41 (m, 1H) |
| 97 | 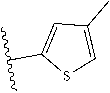 [SMILES: *c1cc(C)cs1] | (R)-N-(1-(4-amino-7-(4- (methylsulfonyl)phenyl) pyrrolo[2,1- f][1,2,4]triazin-5- yl)piperidin-3-yl)-4- methylthiophene-2- carboxamide, TFA | 510.9 | A: 1.466 B: 1.670 | (500 MHz, DMSO-d₆) δ ppm 8.32 (br d, J = 8.5 Hz, 3H), 8.02-7.95 (m, 3H), 7.62 (s, 1H), 7.32 (d, J = 1.2 Hz, 1H), 7.23 (s, 1H), 4.22-4.09 (m, 1H), 3.62-3.52 (m, 1H), 3.23 (s, 3H), 3.12-3.03 (m, 1H), 2.80-2.67 (m, 2H), 2.21 (s, 3H), 1.96-1.79 (m, 3H), 1.61-1.46 (m, 1H) |
| 98 | 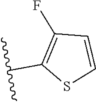 [SMILES: *c1sccc1F] | (R)-N-(1-(4-amino-7-(4- (methylsulfonyl)phenyl) pyrrolo[2,1- f][1,2,4]triazin-5- yl)piperidin-3-yl)-3- fluorothiophene-2- carboxamide | 515.1 | A: 1.597 B: 1.502 | (500 MHz, DMSO-d₆) δ ppm 8.34 (br d, J = 8.5 Hz, 2H), 7.95 (br d, J = 8.5 Hz, 2H), 7.89 (s, 1H), 7.81-7.76 (m, 1H), 7.74 (br t, J = 4.7 Hz, 1H), 7.20 (s, 1H), 7.06 (d. J = 5.5 Hz, 1H), 7.02-6.79 (m, 1H), 4.22-4.08 (m, 1H), 3.21 (s, 3H), 3.03-2.93 (m, 1H), 2.86-2.71 (m, 1H), 1.91-1.70 (m, 3H), 1.66-1.51 (m, 1H) |
| 99 | 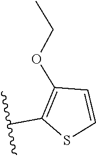 [SMILES: *c1sccc1OCC] | (R)-N-(1-(4-amino-7-(4- (methylsulfonyl)phenyl) pyrrolo[2,1- f][1,2,4]triazin-5- yl)piperidin-3-yl)-3- ethoxythiophene-2- carboxamide | 541.4 | A: 1.496 B: 1.768 | (500 MHz, DMSO-d₆) δ ppm 8.39-8.29 (m, 2H), 8.20- 8.02 (m, 1H), 7.95 (dd, J = 8.5, 1.5 Hz, 2H), 7.92-7.87 (m, 1H), 7.70 (td, J = 3.6, 1.7 Hz, 1H), 7.24 (br d, J = 1.6 Hz, 1H), 7.11 (dd, J = 5.4, 1.6 Hz, 1H), 4.39-4.23 (m, 2H), 4.24-4.15 (m, 1H), 3.57-3.48 (m, 5H), 3.26-3.17 (m, 3H), 1.97-1.72 (m, 3H), 1.64-1.50 (m, 1H), 1.41- 1.30 (m, 3H) |
| 100 | 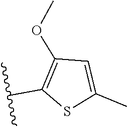 [SMILES: *c1sc(C)cc1OC] | (R)-N-(1-(4-amino-7-(4- (methylsulfonyl)phenyl) pyrrolo[2,1- f][1,2,4]triazin-5- yl)piperidin-3-yl)-3- methoxy-5- methylthiophene-2- carboxamide | 540.9 | A: 1.798 B: 1.519 | (500 MHz, DMSO-d₆) δ ppm 8.35 (br d, J = 8.5 Hz, 2H), 8.18-8.04 (m, 1H), 7.96 (br d, J = 8.5 Hz, 2H), 7.90 (s, 1H), 7.23 (s, 1H), 6.90 (s, 1H), 6.88-6.75 (m, 1H), 4.25- 4.09 (m, 1H), 3.95 (s, 3H), 3.45-3.36 (m, 1H), 3.22 (s, 3H), 2.96-2.73 (m, 1H), 2.41 (s, 3H), 1.88-1.71 (m, 3H), 1.70-1.49 (m, 1H) |
| 101 | 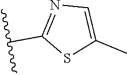 [SMILES: *c1ncc(C)s1] | (R)-N-(1-(4-amino-7-(4- (methylsulfonyl)phenyl) pyrrolo[2,1- f][1,2,4]triazin-5- yl)piperidin-3-yl)-5- methylthiazole-2- carboxamide | 512.1 | A: 1.776 B: 1.423 | (500 MHz, DMSO-d₆) δ ppm 8.65 (br d, J = 8.2 Hz, 1H), 8.36 (br d, J = 8.5 Hz, 2H), 7.95 (br d, J = 8.2 Hz, 2H), 7.90 (s, 1H), 7.69 (s, 1H), 7.20 (s, 1H), 4.23-4.10 (m, 1H), 3.22 (s, 3H), 3.06-2.96 (m, 1H), 2.85-2.68 (m, 1H), 2.54 (s, 3H), 1.93-1.75 (m, 3H), 1.71-1.57 (m, 1H) |
| 102 | 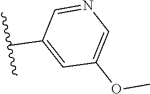 [SMILES: *c1cncc(OC)c1] | (R)-N-(1-(4-amino-7-(4- (methylsulfonyl)phenyl) pyrrolo[2,1- f][1,2,4]triazin-5- yl)piperidin-3-yl)-5- methoxynicotinamide, TFA | 522.1 | A: 1.563 B: 1.185 | (500 MHz, DMSO-d₆) δ ppm 8.60 (br s, 2H), 8.41 (br d, J = 2.7 Hz, 1H), 8.33 (br d, J = 8.6 Hz, 2H), 8.03-7.94 (m, 3H), 7.73 (dd, J = 2.8, 1.8 Hz, 1H), 7.24 (s, 1H), 4.34- 4.18 (m, 1H), 3.87 (s, 3H), 3.62-3.49 (m, 4H), 3.23 (s, 3H), 2.84-2.70 (m, 1H), 2.02-1.77 (m, 3H), 1.64-1.49 (m, 1H) |

### Example 103

### Preparation of (S)-1-(4-amino-7-(4-(methylsulfonyl)phenyl)pyrrolo[2,1-f][1,2,4]triazin-5-yl)-N-(thiazol-2-yl)piperidine-3-carboxamide

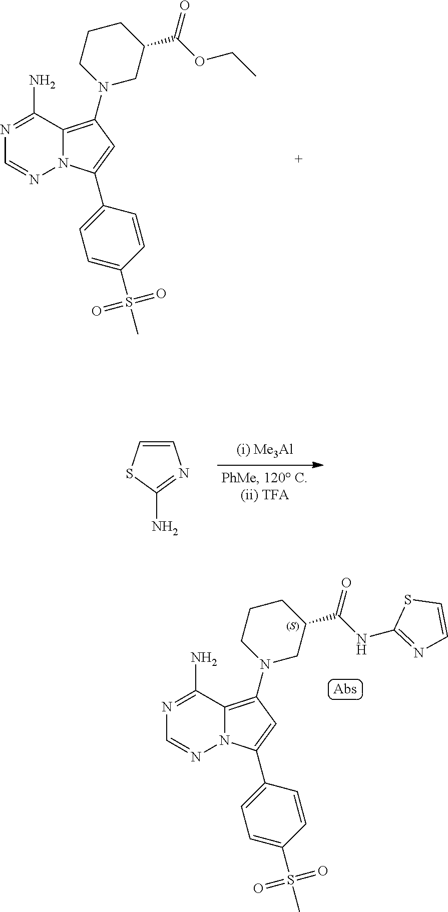

Thiazol-2-amine (4.5 mg, 0.045 mmol) was suspended in anhydrous toluene (1 mL), then trimethylaluminum (2 M in toluene) (0.045 mL, 0.090 mmol) was added dropwise. After stirring for 5 min at rt, Example 1 (10 mg, 0.023 mmol) was added, and the reaction mixture was stirred at 120° C. for 15 min under microwave irradiation. The reaction mixture was cooled to rt, diluted with MeOH (0.5 mL) and carefully quenched with TFA. Solvent was removed under reduced pressure, the residue was diluted with DMF (2 mL), filtered, and purified by preparative HPLC to afford Example 103 (1.3 mg, 9% yield). LC-MS Method A: RT=1.762 min, [M+H]⁺=498.2; LC-MS Method B: RT=1.221 min, [M+H]⁺=498.2; ¹H NMR (500 MHz, DMSO-d₆) δ ppm 8.38 (d, J=8.8 Hz, 3H), 8.31 (t, J=1.5 Hz, 3H), 8.06 (br d, J=8.7 Hz, 2H), 7.99-7.90 (m, 4H), 7.44 (d, J=3.6 Hz, 2H), 7.31 (s, 1H), 7.17 (d, J=3.5 Hz, 2H), 3.90 (d, J=1.0 Hz, 1H), 1.85 (br s, 2H), 1.70 (s, 3H).

### Example 104

### Preparation of (R)-1-(4-amino-7-(4-(methylsulfonyl)phenyl)pyrrolo[2,1-f][1,2,4]triazin-5-yl)-N-(thiazol-2-yl)piperidine-3-carboxamide

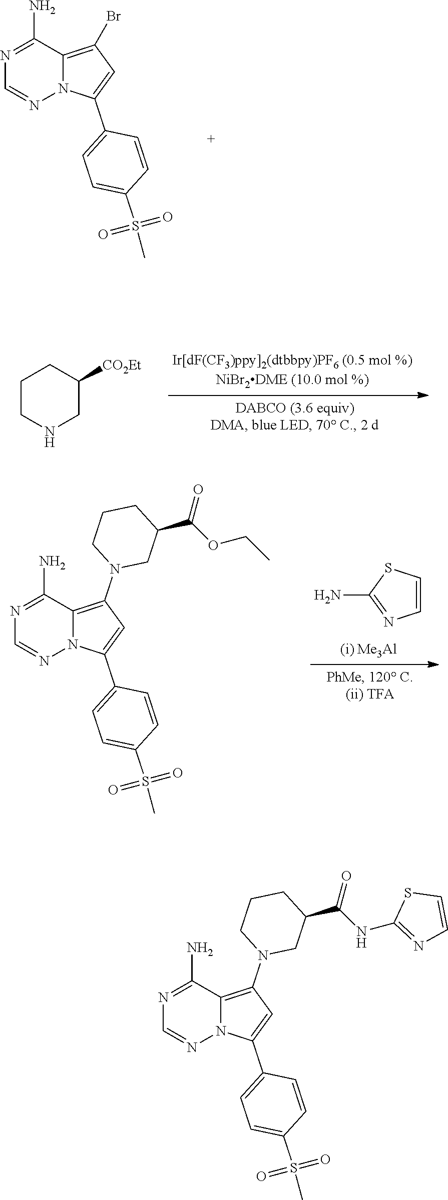 [SMILES: CS(=O)(=O)c1ccc(-c2cc(Br)c3c(N)ncnn23)cc1 ; CCOC(=O)[C@@H]1CCCNC1 ; CCOC(=O)[C@@H]1CCCN(c2cc(-c3ccc(S(C)(=O)=O)cc3)n3ncnc(N)c23)C1 ; Nc1nccs1 ; CS(=O)(=O)c1ccc(-c2cc(N3CCC[C@@H](C(=O)Nc4nccs4)C3)c3c(N)ncnn23)cc1]

### Example 104A. ethyl (R)-1-(4-amino-7-(4-(methylsulfonyl)phenyl)pyrrolo[2,1-f][1,2,4]triazin-5-yl)piperidine-3-carboxylate

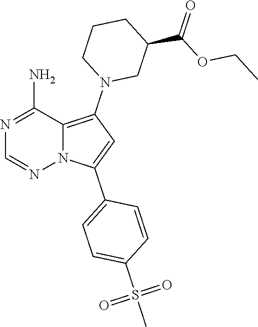 [SMILES: CCOC(=O)[C@@H]1CCCN(c2cc(-c3ccc(S(C)(=O)=O)cc3)n3ncnc(N)c23)C1]

According to the procedure for the preparation of Example 11, reaction of Example 1B and tert-butyl ethyl (R)-piperidine-3-carboxylate afforded Example 104A (113 mg, 62% yield) as a white solid. MS: [M+H]⁺=444.1; ¹H NMR (500 MHz, DMSO-d₆) δ ppm 8.37 (d, J=8.9 Hz, 2H), 7.96 (d, J=8.7 Hz, 2H), 7.91 (s, 1H), 7.29 (s, 1H), 7.23-7.05 (m, 1H), 4.17-4.07 (m, 2H), 3.24 (s, 3H), 3.07-2.94 (m, 2H), 2.93-2.85 (m, 1H), 1.82-1.68 (m, 3H), 1.23 (br s, 1H), 1.19 (t, J=7.1 Hz, 3H).

### Example 104

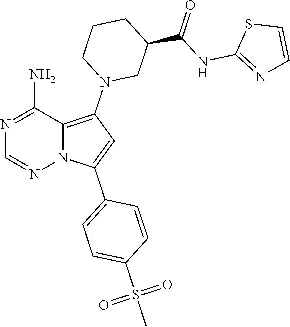 [SMILES: CS(=O)(=O)c1ccc(-c2cc(N3CCC[C@@H](C(=O)Nc4nccs4)C3)c3c(N)ncnn23)cc1]

According to the procedure for the preparation of Example 103, reaction of Example 104A and thiazol-2-amine afforded Example 104 (2.3 mg, 19% yield). LC-MS Method A: RT=1.575 min, [M+H]⁺=498.1; LC-MS Method B: RT=1.322 min, [M+H]⁺=498.1; ¹H NMR (500 MHz, DMSO-d₆) δ ppm. 8.38 (d, J=8.5 Hz, 2H), 8.07 (br d, J=8.5 Hz, 1H), 7.96 (d, J=8.5 Hz, 21H), 7.91 (s, 1H), 7.46 (d, J=3.7 Hz, 1H), 7.31 (s, 1H), 7.21 (d, J=3.7 Hz, 1H), 3.24 (s, 3H), 3.12-3.03 (m, 1H), 2.99 (s, 1H), 2.87-2.71 (m, 1H), 2.01-1.91 (m, 1H), 1.90-1.82 (m, 1H), 1.79-1.62 (m, 2H).

The following Examples in Table 3 were prepared using the same procedure as shown in Example 104. Example 104A was coupled with the appropriate ester.

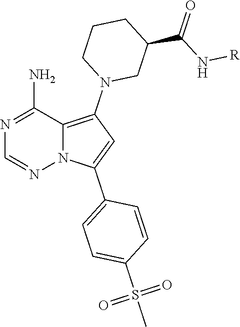

TABLE 3

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 105 | 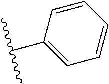 [SMILES: *c1ccccc1] | (R)-1-(4-amino-7-(4- (methylsulfonyl)phenyl)pyrrolo [2,1-f][1,2,4]triazin-5-yl)-N- phenylpiperidine-3-carboxamide | 491.1 | A: 2.073 B: 1.791 | (500 MHz, DMSO-d₆) δ ppm 9.96 (s, 1H), 8.41-8.33 (m, 2H), 8.14-8.01 (m, 1H), 7.96 (d, J = 8.5 Hz, 2H), 7.91 (s, 1H), 7.59 (d, J = 7.6 Hz, 2H), 7.34-7.24 (m, 3H), 7.03 (t, J = 7.5 Hz, 1H), 3.23 (s, 3H), 3.11-3.03 (m, 1H), 2.90-2.71 (m, 1H), 1.97-1.90 (m, 1H), 1.89-1.81 (m, 1H), 1.80-1.61 (m, 2H) |
| 106 | 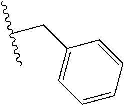 [SMILES: *Cc1ccccc1] | (R)-1-(4-amino-7-(4- (methylsulfonyl)phenyl)pyrrolo [2,1-f][1,2,4]triazin-5-yl)-N- benzylpiperidine-3-carboxamide | 505.1 | A: 1.741 B: 1.348 | (500 MHz, DMSO-d₆) δ ppm 8.42 (br t, J = 5.8 Hz, 1H), 8.36 (d, J = 8.9 Hz, 2H), 8.07-7.98 (m, 1H), 7.96 (d, J = 8.5 Hz, 2H), 7.89 (s, 1H), 7.34-7.25 (m, 3H), 7.24-7.16 (m, 3H), 4.36-4.21 (m, 2H), 3.22 (s, 3H), 3.02 (br dd, J = 4.7, 2.3 Hz, 1H), 2.84-2.72 (m, 1H), 2.69 (br dd, J = 4.1, 1.7 Hz, 1H), 1.94- 1.77 (m, 2H), 1.76-1.59 (m, 2H) |

### Example 107

### Preparation of (R)—N-(1-(4-amino-7-(4-(methylsulfonyl)phenyl)pyrrolo[2,1-f][1,2,4]triazin-yl)piperidin-3-yl)benzenesulfonamide

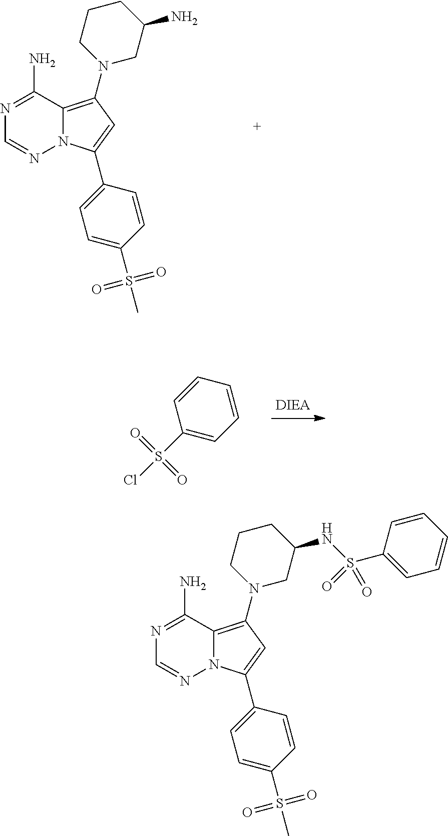 [SMILES: CS(=O)(=O)c1ccc(-c2cc(N3CCC[C@@H](N)C3)c3c(N)ncnn23)cc1 ; O=S(=O)(Cl)c1ccccc1 ; CS(=O)(=O)c1ccc(-c2cc(N3CCC[C@@H](NS(=O)(=O)c4ccccc4)C3)c3c(N)ncnn23)cc1]

Example 13A (10 mg, 0.024 mmol) and DIEA (0.021 mL, 0.118 mmol) were dissolved in anhydrous THF (1.5 mL). Afterwards, benzenesulfonyl chloride (3.9 μl, 0.031 mmol) was added and the reaction mixture was stirred at 0° C. for 15 min, and then at rt for 1 h. The reaction mixture was quenched with MeOH (0.1 mL), and most of the solvent was removed under reduced pressure. The residue was diluted with DMF (2 mL), acidified with TFA (0.1 mL), filtered and purified by preparative HPLC to afford Example 107 (5.7 mg, 45% yield). LC-MS Method A: RT=1.581 min, [M+H]⁺=527.1; LC-MS Method B: RT=1.410 min, [M+H]⁺=527.1; ¹H NMR (500 MHz, DMSO-d₆) δ ppm 8.30 (d, J=8.5 Hz, 2H), 7.94 (d, J=8.6 Hz, 2H), 7.87 (s, 1H), 7.85-7.80 (m, 2H), 7.61-7.48 (m, 3H), 7.07 (br s, 1H), 3.47-3.33 (m, 1H), 2.97-2.82 (m, 1H), 1.84-1.69 (m, 1H), 1.68-1.50 (m, 2H), 1.35-1.20 (m, 1H).

### Example 108

### Preparation of (S)—N-(1-(4-amino-7-(4-(methylsulfonyl)phenyl)pyrrolo[2,1-f][1,2,4]triazin-5-yl)piperidin-3-yl)benzamide

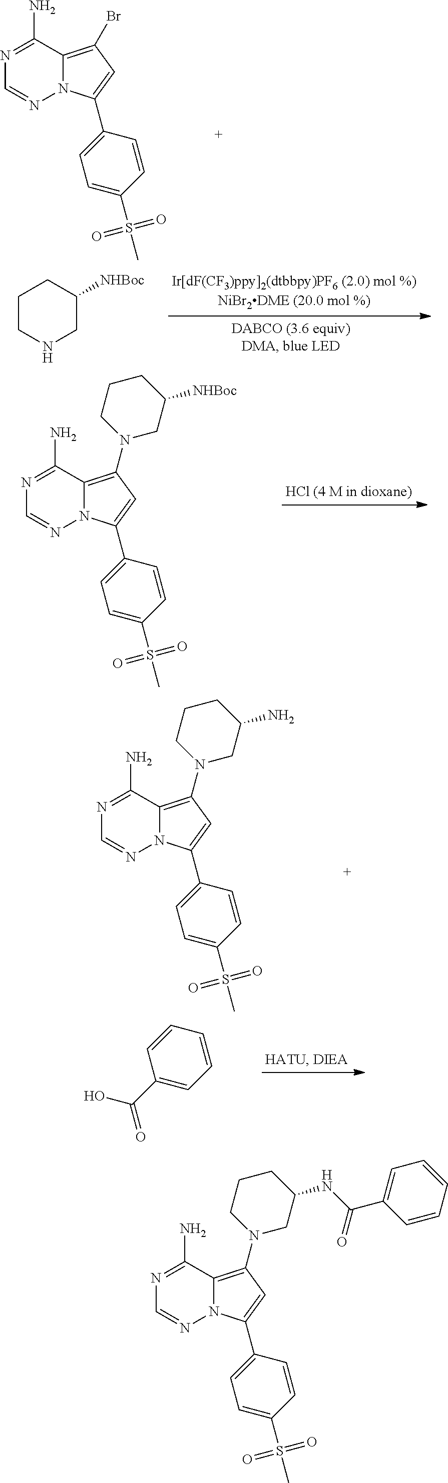

### Example 108A. tert-butyl (S)-(1-(4-amino-7-(4-(methylsulfonyl)phenyl)pyrrolo[2,1-f][1,2,4]triazin-5-yl)piperidin-3-yl)carbamate

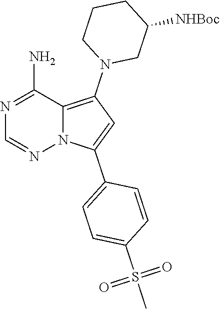 [SMILES: CC(C)(C)OC(=O)N[C@H]1CCCN(c2cc(-c3ccc(S(C)(=O)=O)cc3)n3ncnc(N)c23)C1]

Example 1B (100 mg, 0.272 mmol), Ir[dF(CF₃)ppy]₂(dtbbpy)PF₆ (6.1 mg, 5.5 μmol), tert-butyl (S)-piperidin-3-ylcarbamate (218 mg, 1.09 mmol) and DABCO (110 mg, 0.98 mmol) were placed in a pressure relief vial. The reaction mixture was degassed (3× vacuum/nitrogen), then a solution of NiBr₂-DME (16.8 mg, 0.054 mmol) in DMA (3.3 mL) was added. The reaction mixture was degassed again, capped and stirred under blue LED irradiation with fan cooling at rt for 7 d. The reaction mixture was diluted with EtOAc (200 mL), washed with water (3×50 mL), brine (50 mL), dried (Na₂SO₄) and filtered. EtOAc was removed under reduced pressure, the crude material was purified by flash chromatography (20-100% EtOAc/DCM gradient) to afford Example 108A (105 mg, 53% yield) as an off-white solid. MS: [M+H]⁺=487.2; ¹H NMR (500 MHz, DMSO-d₆) δ ppm 8.43-8.32 (m, 3H), 8.15-8.00 (m, 1H), 7.96 (d, J=8.5 Hz, 2H), 7.91 (s, 1H), 7.86-7.79 (m, 2H), 7.56-7.49 (m, 1H), 7.49-7.41 (m, 2H), 7.22 (s, 1H), 7.10-6.90 (m, 1H), 4.29-4.10 (m, 1H), 3.23 (s, 3H), 3.11-2.99 (m, 1H), 2.84-2.65 (m, 1H), 1.99-1.86 (m, 2H), 1.88-1.71 (m, 1H), 1.67-1.44 (m, 1H).

### Example 108B. (S)-5-(3-aminopiperidin-1-yl)-7-(4-(methylsulfonyl)phenyl)pyrrolo[2,1-f][1,2,4]triazin-4-amine, HCl

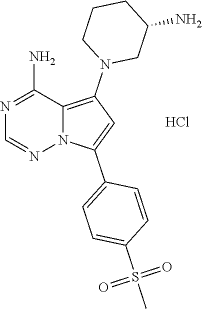 [SMILES: CS(=O)(=O)c1ccc(-c2cc(N3CCC[C@H](N)C3)c3c(N)ncnn23)cc1 ; Cl]

According to the procedure for the preparation of Example 13A, reaction of Example 108A afforded Example 108B (55 mg) as an off-white solid. MS: [M+H]⁺=387.2; ¹H NMR (500 MHz, DMSO-d₆) δ ppm 8.96-8.64 (m, 1H), 8.38-8.26 (m, 51H), 8.07-7.99 (m, 3H), 7.81-7.59 (m, 1H), 7.29 (s, 1H), 3.75-3.64 (m, 2H), 3.62-3.57 (m, 1H), 3.51-3.42 (m, 1H), 3.26 (s, 3H), 2.15-1.96 (m, 1H), 1.92-1.79 (m, 1H), 1.79-1.63 (m, 2H).

### Example 108

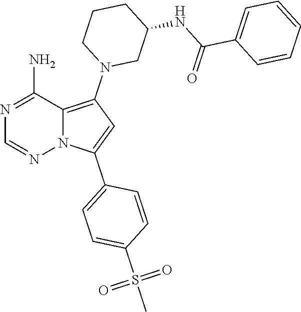 [SMILES: CS(=O)(=O)c1ccc(-c2cc(N3CCC[C@H](NC(=O)c4ccccc4)C3)c3c(N)ncnn23)cc1]

According to the procedure for the preparation of Example 13, reaction of Example 108B and tert-butyl piperidin-4-ylcarbamate afforded Example 108 (3.0 mg, 24% yield). LC-MS Method A: RT=1.421 min, [M+H]⁺=491.0; LC-MS Method B: RT=1.421 min, [M+H]⁺=491.0; ¹H NMR (500 MHz, DMSO-d₅) δ ppm 8.43-8.32 (m, 3H), 8.15-8.00 (m, 1H), 7.96 (d, J=8.5 Hz, 2H), 7.91 (s, 1H), 7.86-7.79 (m, 21H), 7.56-7.49 (m, 1H), 7.49-7.41 (m, 2H), 7.22 (s, 1H), 7.10-6.90 (m, 1H), 4.29-4.10 (m, 1H), 3.23 (s, 3H), 3.11-2.99 (m, 1H), 2.84-2.65 (m, 1H), 1.99-1.86 (m, 2H), 1.88-1.71 (m, 1H), 1.67-1.44 (m, 1H).

The following examples in Table 4 were prepared using the same procedure as shown in Example 108. Example 108B was coupled with the appropriate carboxylic acid. Various coupling reagents could be used other than the one described, such as BOP, PyBop, EDC/HOBt or T3P.

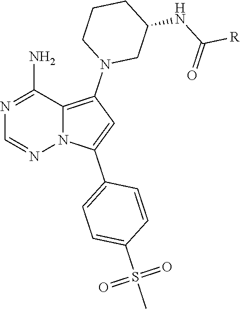

TABLE 4

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 109 | 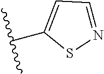 [SMILES: *c1ccns1] | (S)-N-(1-(4-amino-7-(4- (methylsulfonyl)phenyl) pyrrolo[2,1-f] [1,2,4]triazin-5- yl)piperidin-3-yl)isothiazole- 5-carboxamide, TFA | 497.9 | A: 1.453 B: 1.217 | (500 MHz, DMSO-d₆) δ ppm 8.81 (br d, J = 7.6 Hz, 1H), 8.65 (d, J = 1.8 Hz, 1H), 8.38-8.28 (m, 2H), 8.04-7.92 (m, 4H), 7.26 (s, 1H), 4.32-4.09 (m, 1H), 3.33-3.26 (m, 1H), 3.24 (s, 2H), 3.14-3.04 (m, 1H), 2.82-2.71 (m, 1H), 2.03-1.90 (m, 1H), 1.92-1.78 (m, 2H), 1.66-1.47 (m, 1H), 1.27-1.09 (m, 1H) |
| 110 | 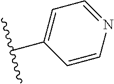 [SMILES: *c1ccncc1] | (S)-N-(1-(4-amino-7-(4- (methylsulfonyl)phenyl) pyrrolo[2,1-f] [1,2,4]triazin-5- yl)piperidin-3- yl)isonicotinamide | 492.0 | A: 1.344 B: 1.030 | (500 MHz, DMSO-d₆) δ ppm 8.36 (d, J = 8.5 Hz, 2H), 8.21- 8.08 (m, 1H), 7.96 (d, J = 8.2 Hz, 2H), 7.92 (s, 1H), 7.75 (d, J = 5.8 Hz, 2H), 7.23 (s, 1H), 4.29-4.18 (m, 1H), 3.23 (s, 3H), 3.11-2.96 (m, 1H), 2.84-2.68 (m, 1H), 1.99-1.86 (m, 2H), 1.89-1.74 (m, 1H), 1.66-1,46 (m, 1H) |
| 111 | 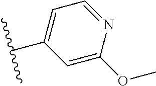 [SMILES: *c1ccnc(OC)c1] | (S)-N-(1-(4-amino-7-(4- (methylsulfonyl)phenyl) pyrrolo[2,1-f] [1,2,4]triazin-5- yl)piperidin-3-yl)-2- methoxyisonicotinamide | 522.0 | A: 1.515 B: 1.273 | (500 MHz, DMSO-d₆) δ ppm 8.61 (br d, J = 7.6 Hz, 1H), 8.36 (d, J = 8.1 Hz, 2H), 8.27 (d, J = 5.2 Hz, 1H), 8.16-8.00 (m, 1H), 7.96 (d, J = 8.0 Hz, 2H), 7.91 (s, 1H), 7.35-7.30 (m, 1H), 7.22 (s, 1H), 7.18 (s, 1H), 4.26-4.13 (m, 1H), 3.88 (d, J = 0.8 Hz, 3H), 3.56-3.39 (m, 1H), 3.23 (s, 3H), 3.10-3.01 (m, 1H), 2.84-2.66 (m, 1H), 2.02-1.85 (m. 2H), 1.85-1.73 (m, 1H), 1.60-1.45 (m, 1H). 1.20 (br t. J = 7.2 Hz, 1H) |

### Example 112

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)isothiazole-5-carboxamide, TFA

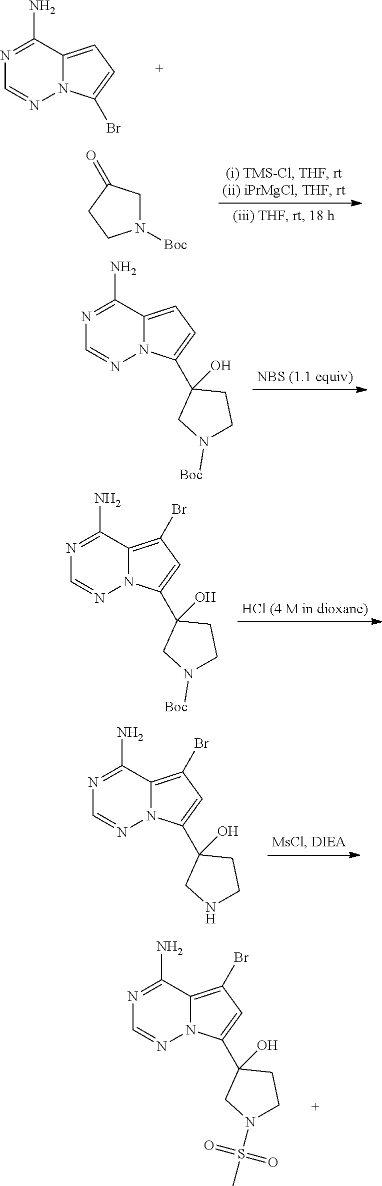

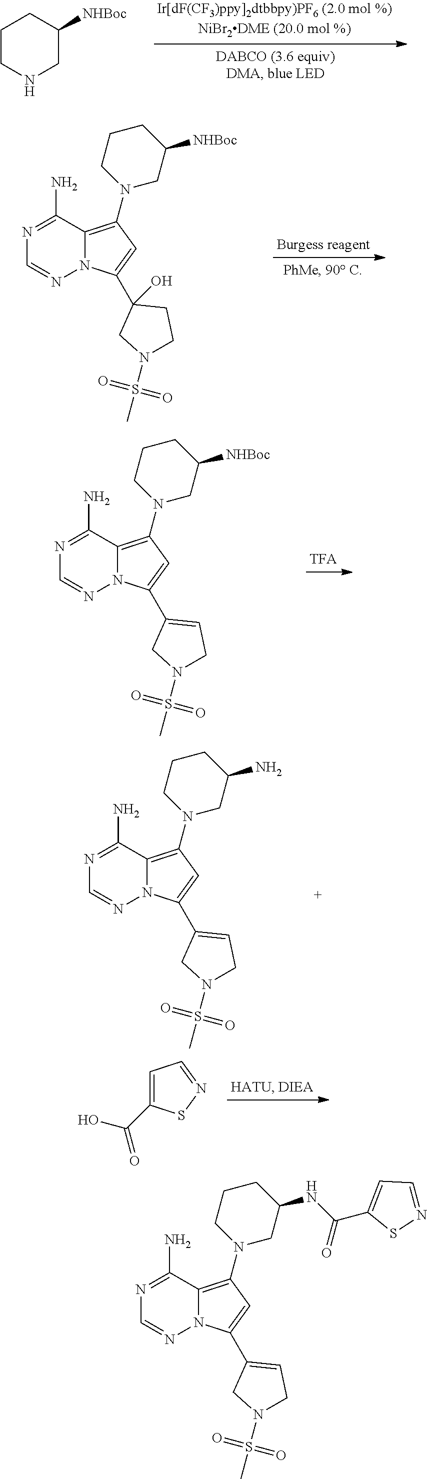

### Example 112A. tert-Butyl 3-(4-aminopyrrolo[2,1-f][1,2,4]triazin-7-yl)-3-hydroxypyrrolidine-1-carboxylate

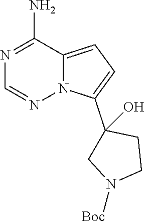 [SMILES: CC(C)(C)OC(=O)N1CCC(O)(c2ccc3c(N)ncnn23)C1]

7-Bromopyrrolo[2,1-f][1,2,4]triazin-4-amine (1000 mg, 4.69 mmol) was suspended in anhydrous THF (25 mL), and TMS-Cl (1.50 mL, 11.7 mmol) was added. The reaction mixture was stirred at rt for 12 h. Afterwards, iPrMgCl (2 M in THF) (12.3 mL, 24.6 mmol) was added dropwise over 5 min (slight exotherm, water bath is used), resulting in a clear solution. The reaction mixture was stirred at rt for 3 h, and tert-butyl 3-oxopyrrolidine-1-carboxylate (2174 mg, 11.7 mmol) was added portionwise. The reaction mixture was stirred at rt for 16 h. The reaction mixture was poured over a mixture of ice (~200 g) and saturated aqueous NH₄Cl (150 mL). The mixture was allowed to warm to rt with stirring, then was extracted with EtOAc (3×100 mL). The combined organic layers were washed with brine (100 mL), dried (Na₂SO₄), and concentrated. The residue was purified by flash chromatography (1-10% MeOH/DCM gradient) to give Example 112A (945 mg, 63% yield) as an off-white solid. MS: [M+H]⁺=320.2; ¹H NMR (500 MHz, DMSO-d₆) δ ppm 7.84 (d, J=4.9 Hz, 1H), 7.67 (br s, 2H), 6.84 (d, J=4.4 Hz, 1H), 6.62 (d, J=4.4 Hz, 1H), 5.52 (d, J=18.8 Hz, 1H), 3.79 (dd, J=13.1, 11.3 Hz, 1H), 3.64-3.52 (m, 1H), 3.51-3.38 (m, 2H), 2.71-2.55 (m, 1H), 2.13-1.99 (m, 1H), 1.41 (d, J=13.7 Hz, 9H).

### Example 112B. tert-Butyl 3-(4-amino-5-bromopyrrolo[2,1-f][1,2,4]triazin-7-yl)-3-hydroxypyrrolidine-1-carboxylate

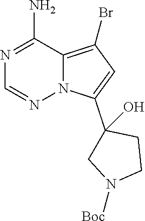 [SMILES: CC(C)(C)OC(=O)N1CCC(O)(c2cc(Br)c3c(N)ncnn23)C1]

To a solution of Example 112A (945 mg, 2.96 mmol) in DMF (30 mL) at rt, was added NBS (579 mg, 3.25 mmol). The mixture was stirred at rt protected from light for 1 h. The reaction mixture was added into well-stirred 245 mL half-saturated aq. NaHCO₃ and ~13 mL of aq. sat. Na₂SO₃ was added. The resultant suspension was stirred at rt for 2 h, then was filtered, The collected solid was rinsed with H₂O (5×5.0 mL). The solid was dried in vacuo to afford Example 112B (1005 mg, 85% yield) as a white solid. MS: [M+H]⁺=398.0; ¹H NMR (500 MHz, DMSO-d₆) δ ppm 7.89 (d, J=5.2 Hz, 1H), 6.76 (d, J=0.6 Hz, 1H), 5.64 (d, J=15.1 Hz, 1H), 3.77 (dd, J=15.6, 11.2 Hz, 1H), 3.66-3.52 (m, 1H), 3.50-3.36 (m, 2H), 2.67-2.53 (m, 1H), 2.13-1.98 (m, 1H), 1.40 (d, J=13.0 Hz, 9H).

### Example 112C. 3-(4-amino-5-bromopyrrolo[2,1-f][1,2,4]triazin-7-yl)pyrrolidin-3-ol, HCl

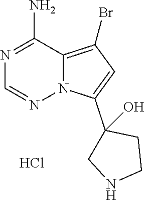 [SMILES: Nc1ncnn2c(C3(O)CCNC3)cc(Br)c12 ; Cl]

To a mixture of Example 112B (300 mg, 0.753 mmol) in DCM (10 mL), was added HCl (4 M in dioxane) (5.0 mL, 20.00 mmol). The reaction mixture was stirred at rt for 1 h. Solvent was removed under reduced pressure, the residue was co-evaporated with Et₂O (3×), and dried under vacuum to afford Example 112C (260 mg) as an off-white solid. MS: [M+H]⁺=298.1; ¹H NMR (500 MHz, DMSO-d₆) δ ppm 9.84-9.68 (m, 1H), 9.61-9.44 (m, 1H), 8.06 (s, 1H), 6.97 (s, 1H), 3.64-3.57 (m, 1H), 3.53-3.48 (m, 1H), 3.47-3.34 (m, 2H), 2.58-2.51 (m, 1H), 2.37-2.29 (m, 1H).

### Example 112D. 3-(4-Amino-5-bromopyrrolo[2,1-f][1,2,4]triazin-7-yl)-1-(methylsulfonyl)pyrrolidin-3-ol

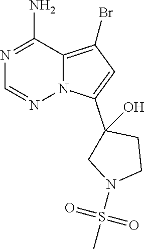 [SMILES: CS(=O)(=O)N1CCC(O)(c2cc(Br)c3c(N)ncnn23)C1]

To a mixture of Example 112C (260 mg, 0.777 mmol) and DIEA (0.679 mL, 3.89 mmol) in THF (15 mL) at 0° C., and, was added Ms-Cl (0.067 mL, 0.855 mmol). The reaction mixture was stirred at 0° C. for 1 h, and then at rt for 1 h. The reaction mixture was quenched with water (1.0 mL), and most of THF was removed under reduced pressure. The residue was diluted with EtOAc (150 mL) and water (100 mL), organic phase was separated, washed with brine, dried (Na₂SO₄), and filtered. EtOAc was removed under reduced pressure to afford Example 112D (252 mg, 86% yield) as an off-white solid. MS: [M+H]⁺=375.9; ¹H NMR (500 MHz, DMSO-d₆) δ ppm 7.90 (s, 1H), 6.80 (s, 1H), 5.78 (s, 1H), 3.79 (d, J=11.3 Hz, 1H), 3.62 (dd, J=1.1, 1.5 Hz, 1H), 3.51-3.41 (m, 2H), 2.92 (s, 3H), 2.68-2.55 (m, 1H), 2.18 (ddt, J=12.6, 5.5, 1.9 Hz, 1H).

### Example 112E. tert-Butyl ((3R)-1-(4-amino-7-(3-hydroxy-1-(methylsulfonyl)pyrrolidin-3-yl)pyrrolo[2,1-f][1,2,4]triazin-5-yl)piperidin-3-yl)carbamate

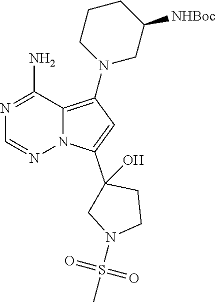 [SMILES: CC(C)(C)OC(=O)N[C@@H]1CCCN(c2cc(C3(O)CCN(S(C)(=O)=O)C3)n3ncnc(N)c23)C1]

Example 112D (150 mg, 0.399 mmol), Ir[dF(CF₃)ppy]₂(dtbbpy)PF₆ (8.9 mg, 8.0 μmol), tert-butyl (R)-piperidin-3-ylcarbamate (319 mg, 1.60 mmol) and DABCO (161 mg, 1.44 mmol) were placed in a pressure relief vial. The reaction mixture was degassed (3× vacuum/nitrogen), then a solution of NiBr₂-DME (24.6 mg, 0.080 mmol) in DMA (4 mL) was added. The reaction mixture was degassed again, capped and stirred under blue LED irradiation with fan cooling at rt for 7 d. The reaction mixture was diluted with EtOAc (200 mL), washed with water (3×50 mL), brine (1×50 mL), dried (Na₂SO₄) and filtered. EtOAc was removed under reduced pressure, the crude material was purified was purified by flash chromatography (50-100% EtOAc/DCM gradient) to give Example 112E (100 mg, 51% yield) as an amber film. MS: [M+H]⁺=496.1 H NMR (500 MHz, DMSO-d₅) δ ppm 7.75 (s, 1H), 6.57 (s, 1H), 5.67 (d, J=1.5 Hz, 1H), 3.80 (d, J=11.0 Hz, 1H), 3.68-3.59 (m, 1H), 3.58 (dd, J=11.1, 1.5 Hz, 1H), 3.50-3.40 (m, 2H), 3.28 (s, 1H), 3.09-2.98 (m, 1H), 2.91 (s, 3H), 2.87-2.79 (m, 1H), 2.70-2.56 (m, 2H), 2.21-2.11 (m, 1H), 1.87-1.72 (m, 2H), 1.69-1.56 (m, 1H), 1.38 (s, 9H), 1.34-1.20 (m, 3H).

### Example 112F. tert-Butyl (R)-(1-(4-amino-7-(1-(methylsulfonyl)-25-dihydro-1H-pyrrol-3-yl)pyrrolo[2,1-f][1,2,4]triazin-5-yl)piperidin-3-yl)carbamate

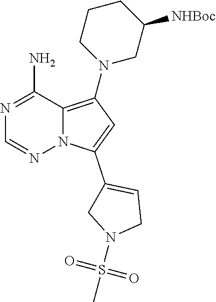 [SMILES: CC(C)(C)OC(=O)N[C@@H]1CCCN(c2cc(C3=CCN(S(C)(=O)=O)C3)n3ncnc(N)c23)C1]

To a mixture of Example 112E (50 mg, 0.101 mmol) in anhydrous toluene (5.0 mL) at it, was added (methoxycarbonylsulfamoyl)triethylammonium hydroxide, inner salt (26.4 mg, 0.111 mmol). The reaction mixture was stirred at 90° C. for 4 h under nitrogen atmosphere. Additional (methoxycarbonylsulfamoyl)triethylammonium hydroxide, inner salt (26.4 mg, 0.111 mmol) was added, and the reaction mixture was stirred at 90° C. for 2 h. The reaction mixture was cooled to rt, quenched with MeOH (2.5 mL), concentrated, and the residue was purified by flash chromatography (50-100% EtOAc/DCM gradient) to afford Example 112F (34 mg, 71% yield) as a white solid. MS: [M+H]⁺=478.1; ¹H NMR (500 MHz, DMSO-d₆) δ ppm 7.86 (s, 1H), 6.74-6.69 (m, 2H), 4.57-4.50 (m, 2H), 4.29 (td, J=4.3, 2.4 Hz, 2H), 3.69-3.57 (m, 1H), 3.28 (s, 2H), 3.02 (s, 2H), 2.95 (s, 3H), 1.85-1.57 (m, 4H), 1.38 (s, 9H), 1.24 (br s, 2H).

### Example 112G. (R)-5-(3-Aminopiperidin-1-yl)-7-(1-(methylsulfonyl)-2,5-dihydro-1H-pyrrol-3-yl)pyrrolo[2,1-f][1,2,4]triazin-4-amine, TFA

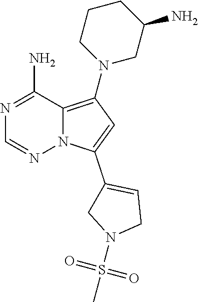 [SMILES: CS(=O)(=O)N1CC=C(c2cc(N3CCC[C@@H](N)C3)c3c(N)ncnn23)C1]

To a solution of Example 112F (34 mg, 0.071 mmol) in anhydrous DCM (2.0 mL) at rt, was added TFA (1.0 mL). The reaction mixture was stirred for 30 min at rt under nitrogen atmosphere. Solvent was removed under reduced pressure, the residue was co-evaporated with Et₂O (2×), and dried under reduced pressure to afford Example 112G (37 mg) as an off-white solid. MS: [M+H]⁺=378.1; ¹H NMR (500 MHz, DMSO-d₆) δ ppm 8.01-7.92 (m, 3H), 7.89 (s, 1H), 6.76 (s, 1H), 6.73 (t, J=2.1 Hz, 1H), 4.56-4.51 (m, 2H), 4.33-4.27 (m, 2H), 3.82-3.75 (m, 3H), 3.60-3.53 (m, 3H), 2.96 (s, 3H), 2.04-1.91 (m, 3H).

### Example 112

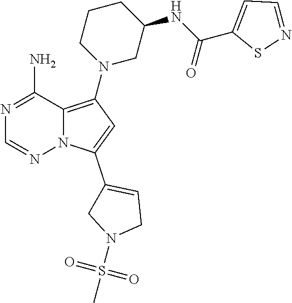 [SMILES: CS(=O)(=O)N1CC=C(c2cc(N3CCC[C@@H](NC(=O)c4ccns4)C3)c3c(N)ncnn23)C1]

Example 112G (12 mg, 0.024 mmol), isothiazole-5-carboxylic acid (4.1 mg, 0.032 mmol) and DIEA (0.021 mL, 0.122 mmol) were suspended in anhydrous DMF (1.5 mL). Afterwards, HATU (12.07 mg, 0.032 mmol) was added and the reaction mixture was stirred at rt for 16 h. The reaction mixture was quenched with MeOH (0.1 mL), acidified with TFA (0.1 mL), diluted with DMF to 2 mL, filtered and purified by preparative HPLC to afford Example 112 (2.6 mg, 18% yield). LC-MS Method A: RT=1.314 min, [M+H]⁺=489.3; LC-MS Method B: RT=1.025 min, [M+H]⁺=489.3; ¹H NMR (500 MHz, DMSO-d₆) δ ppm. 8.81 (br d, J=7.6 Hz, 1H), 8.64 (s, 1H), 7.94 (s, 1H), 7.89 (s, 1H), 6.76 (s, 1H), 6.71 (br d, J=1.5 Hz, 1H), 4.53 (br s, 2H), 4.29 (br s, 2H), 4.20-4.07 (m, 1H), 3.40 (s, 2H), 3.16 (s, 1H), 3.05-2.97 (m, 1H), 2.94 (s, 3H), 2.74-2.62 (m, 2H), 2.02-1.89 (m, 1H), 1.89-1.72 (m, 2H), 1.60-1.42 (m, 1H).

### Example 113

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)benzamide, TFA

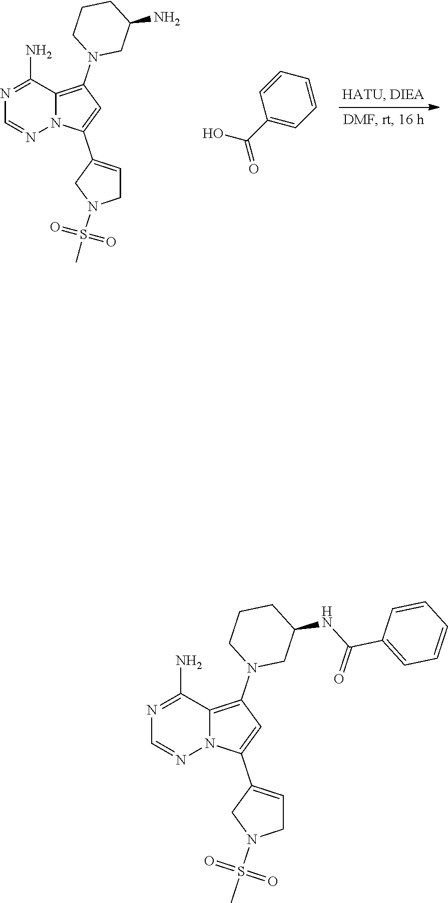 [SMILES: CS(=O)(=O)N1CC=C(c2cc(N3CCC[C@@H](N)C3)c3c(N)ncnn23)C1 ; O=C(O)c1ccccc1 ; CS(=O)(=O)N1CC=C(c2cc(N3CCC[C@@H](NC(=O)c4ccccc4)C3)c3c(N)ncnn23)C1]

According to the procedure for the preparation of Example 112, reaction of Example 112G and benzoic acid afforded Example 113 (2.9 mg, 20% yield). LC-MS Method A: RT=1.309 min, [M+H]⁺=482.0; LC-MS Method B: RT=1.541 min, [M+H]⁺=482.0; ¹H NMR (500 MHz, DMSO-d₆) δ ppm 8.38 (br d, J=7.4 Hz, 1H), 7.85 (s, 1H), 7.83-7.77 (m, 2H), 7.55-7.49 (m, 1H), 7.47-7.41 (m, 2H), 6.75-6.67 (m, 2H), 4.52 (br s, 2H), 4.28 (br s, 2H), 4.21-4.10 (m, 1H), 3.63-3.48 (m, 2H), 3.16 (s, 1H), 2.98 (br s, 1H), 2.93 (s, 3H), 2.73-2.61 (m, 1H), 1.95-1.82 (m, 2H), 1.82-1.71 (m, 1H), 1.59-1.44 (m, 1H).

### Example 114

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-2-methoxyisonicotinamide

### Example 115

### Preparation of (R)—N-(1-(4-amino-7-(1-(methylsulfonyl)-4,5-dihydro-1H-pyrrol-3-yl)pyrrolo[2,1-f][1,2,4]triazin-5-yl)piperidin-3-yl)-2-methoxyisonicotinamide

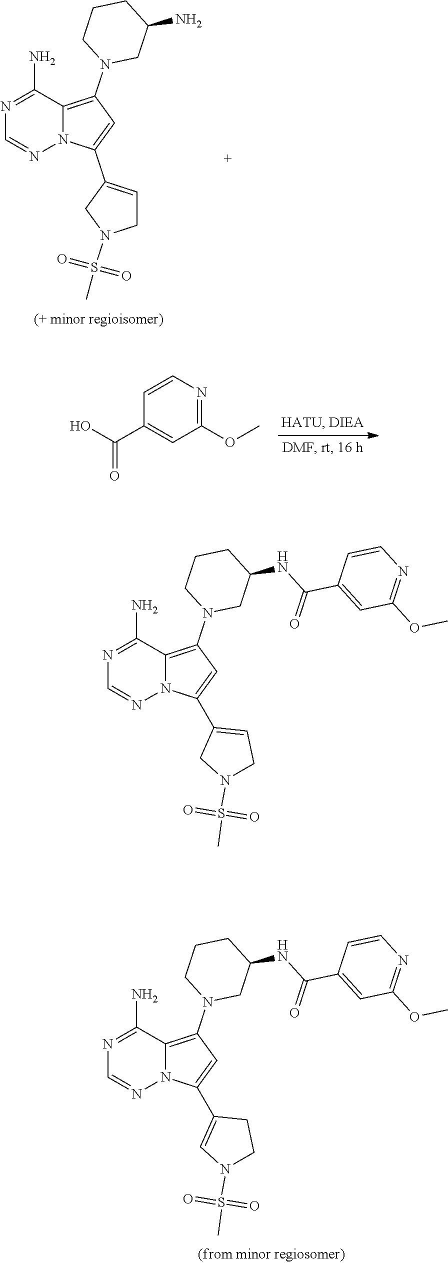 [SMILES: CS(=O)(=O)N1CC=C(c2cc(N3CCC[C@@H](N)C3)c3c(N)ncnn23)C1 ; COc1cc(C(=O)O)ccn1 ; COc1cc(C(=O)N[C@@H]2CCCN(c3cc(C4=CCN(S(C)(=O)=O)C4)n4ncnc(N)c34)C2)ccn1 ; COc1cc(C(=O)N[C@@H]2CCCN(c3cc(C4=CN(S(C)(=O)=O)CC4)n4ncnc(N)c34)C2)ccn1]

According to the procedure for the preparation of Example 112, reaction of Example 112G (+minor dihydropyrrole isomer) and 2-methoxisonicotinic acid afforded Example 114 (1.1 mg, 8.5% yield). LC-MS Method A: RT=1.094 min, [M+H]⁺=513.4; LC-MS Method B: RT=1.391 mt, [M+H]⁺=513.4; ¹H NMR (500 MHz, DMSO-d₆) δ ppm 8.58 (br d, J=7. Hz, 1H), 8.27 (d, J=5.2 Hz, 1H), 7.86 (s, 1H), 7.32 (dd, J=5.2, 1.2 Hz, 1H), 7.16 (s, 1H), 6.75-6.66 (m, 2H), 4.52 (br s, 2H), 4.28 (br d, J=3.7 Hz, 2H), 4.20-4.04 (m, 1H), 3.89 (d, J=6.1 Hz, 3H), 3.44-3.36 (m, 1H), 3.20-3.12 (m, 1H), 2.94 (s, 3H), 2.74-2.60 (m, 1H), 1.93-1.82 (m, 2H), 1.83-1.72 (m, 1H), 1.61-1.43 (m, 2H).

The minor isomer was isolated as Example 115. MS: [M+H]⁺=513.2; HPLC Method C: RT=7.50 min; HPLC Method D. RT=4.67 min; ¹H NMR (500 MHz, DMSO-d₆) δ ppm 8.57 (br d, J=7.8 Hz, 1H), 8.27 (d, J=5.3 Hz, 1H), 7.96 (s, 1H), 7.37 (t, J=1.8 Hz, 1H), 7.33 (dd, J=5.3, 1.4 Hz, 1H), 7.17 (d, J=1.2 Hz, 1H), 6.62 (s, 1H), 4.26-4.15 (m, 1H), 3.89 (s, 31H), 3.79 (br t, J=9.3 Hz, 2H), 3.23-3.16 (m, 2H), 3.10-3.05 (m, 2H), 3.04 (s, 3H), 3.03-2.97 (m, 1H), 2.74-2.65 (m, 2H), 1.95-1.78 (m, 3H).

### Example 116

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-2-methylisonicotinamide

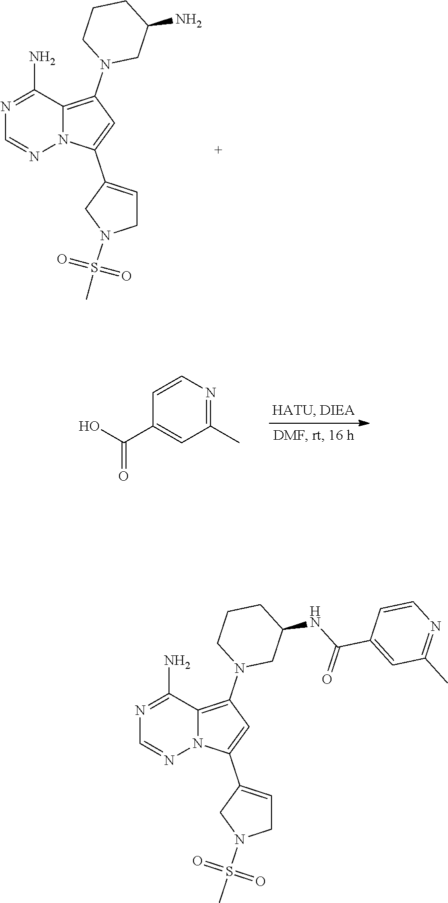

According to the procedure for the preparation of Example 112, reaction of Example 112G and 2-methylisonicotinic acid afforded Example 116 (1.1 mg, 10% yield). LC-MS Method A: RT=1.378 min, [M+H]⁺=497.0; LC-MS Method B: RT=1.004 min, [M+H]⁺=497.0; ¹H NMR (500 MHz, DMSO-d₆) δ ppm 8.64-8.58 (m, 1H), 8.55 (d, J=4.7 Hz, 1H), 7.86 (s, 1H), 7.59 (s, 1H), 7.52 (br d, J=4.9 Hz, 1H), 6.76-6.68 (m, 2H), 4.59-4.51 (m, 2H), 4.33-4.25 (m, 2H), 4.23-4.09 (m, 1H), 2.94 (s, 3H), 2.75-2.63 (m, 1H), 1.96-1.84 (m, 2H), 1.83-1.72 (m, 1H), 1.63-1.46 (m, 1H), 1.31-1.14 (m, 2H).

### Example 117

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-2-fluoroisonicotinamide, TFA

### Example 118

### Preparation of (R)—N-(1-(4-amino-7-(1-(methylsulfonyl)-4,5-dihydro-1H-pyrrol-3-yl)pyrrolo[2,1-f][1,2,4]triazin-5-yl)piperidin-3-yl)-2-fluoroisonicotinamide, TFA

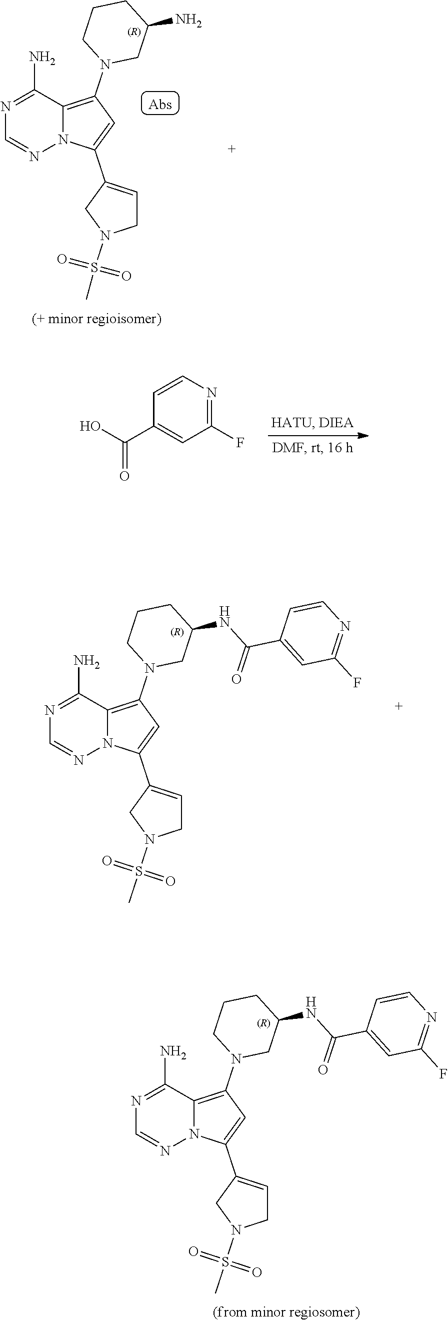

According to the procedure for the preparation of Example 112, reaction of Example 112G (+minor dihydropyrrole regioisomer) and 2-fluoroisonicotinic acid afforded the following analogs.

Example 117 (2.5 mg, 19% yield). LC-MS Method A: RT=1.420 min, [M+H]⁺=501.3; LC-MS Method B: RT=1.208 min, [M+H]⁺=501.3; ¹H NMR (500 MHz, DMSO-d₆) δ ppm 8.77 (br d, J=8.2 Hz, 1H), 8.39 (d, J=5.2 Hz, 1H), 7.93 (s, 1H), 7.75-7.68 (m, 1H), 7.52 (s, 1H), 6.79 (s, 1H), 6.74 (br s, 1H), 4.54 (br d, J=2.6 Hz, 2H), 4.30 (br s, 2H), 4.26-4.13 (m, 1H), 3.24-3.16 (m, 1H), 2.96 (s, 3H), 2.78-2.65 (m, 2H), 1.99-1.78 (m, 3H), 1.62-1.47 (m, 1H).

Example 118 (1.1 mg, 9% yield). LC-MS Method A: RT=1.459 min, [M+H]⁺=501.3; LC-MS Method B: RT=1.208 min, [M+H]⁺=501.3; ¹H NMR (500 MHz, DMSO-d₆) δ ppm 8.76-8.68 (m, 1H), 8.38 (d, J=5.2 Hz, 1H), 7.93 (s, 1H), 7.73-7.69 (m, 1H), 7.52 (s, 1H), 7.36 (s, 1H), 6.60 (s, 1H), 4.27-4.15 (m, 2H), 3.81-3.74 (m, 2H), 3.07 (br d, J=2.4 Hz, 2H), 3.03 (s, 3H), 2.75-2.65 (m, 2H), 1.97-1.76 (m, 4H), 1.58-1.46 (m, 2H).

### Example 119

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-3-isopropylisoxazole-5-carboxamide, TFA

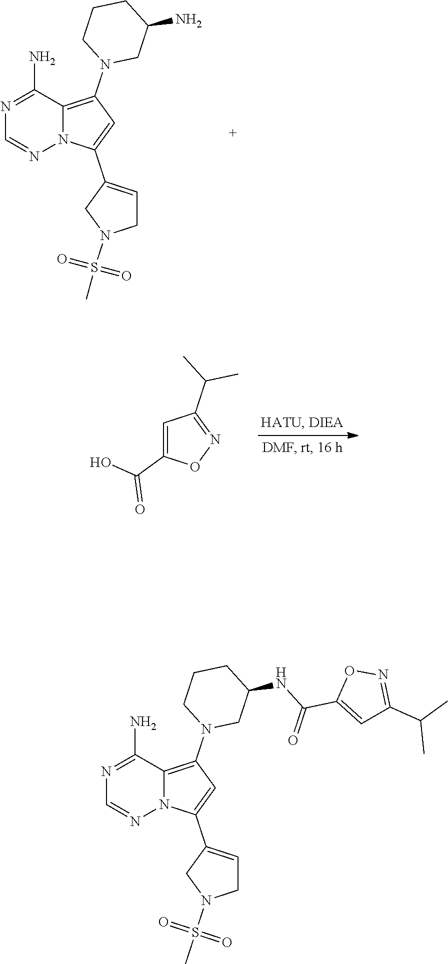 [SMILES: CS(=O)(=O)N1CC=C(c2cc(N3CCC[C@@H](N)C3)c3c(N)ncnn23)C1 ; CC(C)c1cc(C(=O)O)on1 ; CC(C)c1cc(C(=O)N[C@@H]2CCCN(c3cc(C4=CCN(S(C)(=O)=O)C4)n4ncnc(N)c34)C2)on1]

According to the procedure for the preparation of Example 112, reaction of Example 112G and 3-isopropylisoxazole-5-carboxylic acid afforded Example 119 (1.2 mg, 9% yield). LC-MS Method A: RT=1.758 min, [M+H]⁺=515.1; LC-MS Method B: RT=1.475 min, [M+H]⁺=515.1; ¹H NMR (500 MHz, DMSO-d₆) δ ppm 8.89-8.80 (m, 1H), 7.88 (s, 1H), 7.05 (br d, J=2.2 Hz, 1H), 6.74 (s, 1H), 6.71 (br s, 1H), 4.52 (br d, J=3.7 Hz, 2H), 4.32-4.25 (m, 2H), 4.22-4.10 (m, 1H), 3.18-3.10 (m, 1H), 3.08-3.01 (m, 1H), 2.94 (s, 3H), 2.72-2.60 (m, 2H), 1.91-1.75 (m, 3H), 1.58-1.48 (m, 1H), 1.23 (d, J=6.9 Hz, 6H).

### Example 120

### Preparation of tert-butyl (R)-(1-(4-amino-7-(4-(methylsulfonyl)phenyl)pyrrolo[2,1-f][1,2,4]triazin-5-yl)azepan-3-yl)carbamate

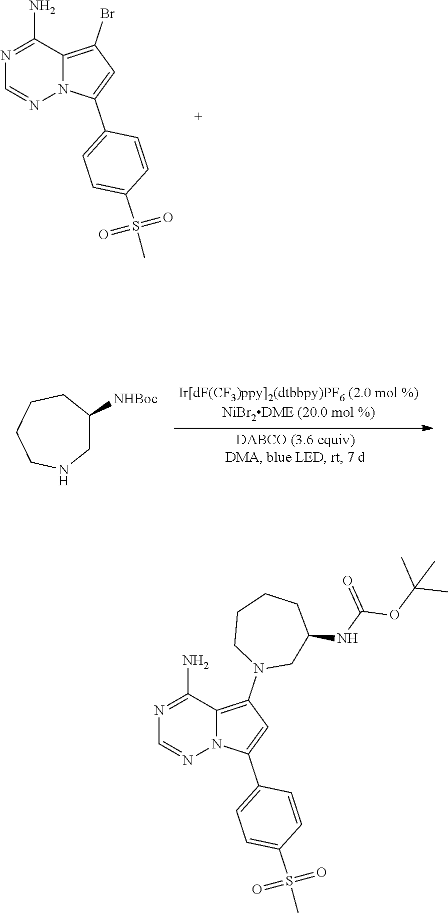 [SMILES: CS(=O)(=O)c1ccc(-c2cc(Br)c3c(N)ncnn23)cc1 ; CC(C)(C)OC(=O)N[C@@H]1CCCCNC1 ; CC(C)(C)OC(=O)N[C@@H]1CCCCN(c2cc(-c3ccc(S(C)(=O)=O)cc3)n3ncnc(N)c23)C1]

According to the procedure for the preparation of Example 11, reaction of Example 1B and tert-butyl (R)-azepan-3-ylcarbamate afforded Example 120 (2.1 mg, 5% yield). LC-MS Method A: RT=1.840 min, [M+H]+=501.3; LC-MS Method B: RT=1.522 min, [M+H]⁺=501.3; 1H NMR (500 MHz, DMSO-d₆) δ ppm 8.32 (d, J=8.5 Hz, 2H), 7.94 (d, J=8.9 Hz, 2H), 7.86 (s, 1H), 7.17 (s, 1H), 6.83 (br d, J=7.0 Hz, 1H), 3.20 (s, 3H), 3.18-3.10 (m, 2H), 3.09-3.00 (m, 2H), 1.94-1.84 (m, 1H), 1.81-1.62 (m, 4H), 1.62-1.48 (m, 2H), 1.29 (br s, 9H).

### Example 121

### Preparation of (R)—N-(1-(4-amino-7-(4-(methylsulfonyl)phenyl)pyrrolo[2,1-f][1,2,4]triazin-5-yl)piperidin-3-yl)-2-methoxy-N-methylisonicotinamide

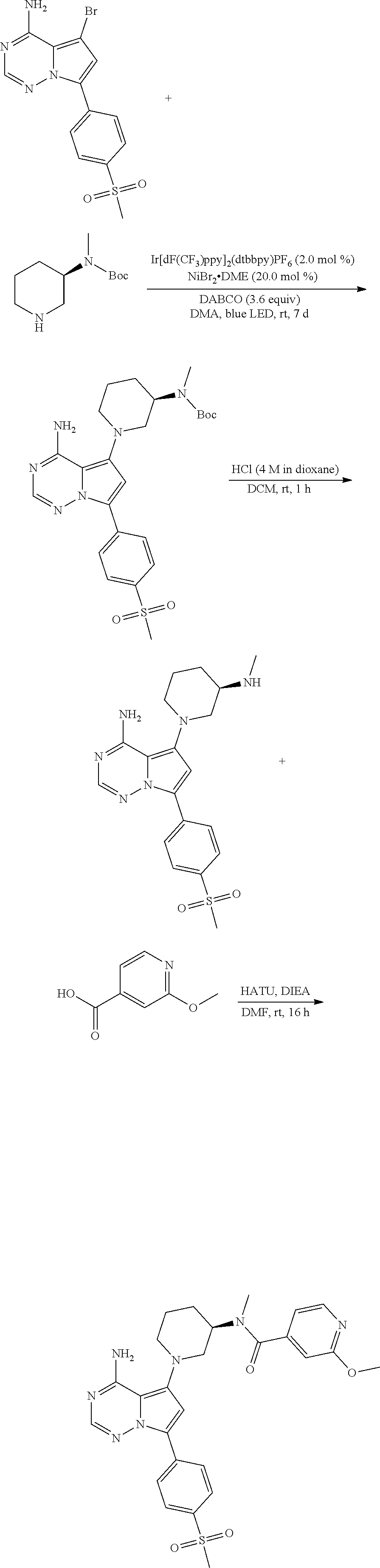

### Example 121A. tert-butyl (R)-(1-(4-amino-7-(4-(methylsulfonyl)phenyl)pyrrolo[2,1-f][1,2,4]triazin-5-yl)piperidin-3-yl)(methyl)carbamate

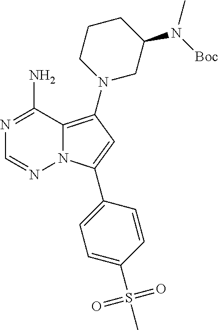 [SMILES: CN(C(=O)OC(C)(C)C)[C@@H]1CCCN(c2cc(-c3ccc(S(C)(=O)=O)cc3)n3ncnc(N)c23)C1]

According to the procedure for the preparation of Example 11, reaction of Example 1B and tert-butyl (R)-methyl(piperidin-3-yl)carbamate afforded Example 121A (35 mg, 17% yield) as a colorless film. MS: [M+H]⁺=501.2; ¹H NMR (500 MHz, DMSO-d₆) δ ppm 8.40-8.34 (m, 2H), 7.97 (d, J=8.9 Hz, 2H), 7.91 (s, 1H), 7.28 (s, 1H), 7.05-6.83 (m, 1H), 3.24 (s, 3H), 3.10-3.01 (m, 1H), 3.02-2.94 (m, 1H), 2.91-2.83 (m, 1H), 2.76 (s, 3H), 1.87-1.70 (m, 4H), 1.68-1.56 (m, 1H), 1.40 (s, 9H).

### Example 121B. (R)-5-(3-(methylamino)piperidin-1-yl)-7-(4-(methylsulfonyl)phenyl)pyrrolo[2,1-f][1,2,4]triazin-4-amine, HCl

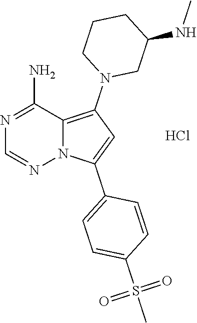 [SMILES: CN[C@@H]1CCCN(c2cc(-c3ccc(S(C)(=O)=O)cc3)n3ncnc(N)c23)C1 ; Cl]

According to the procedure for the preparation of Example 13A, reaction of Example 121A afforded Example 121B (30 mg, 98% yield) as an off-white solid. MS: [M+H]⁺=401.2; ¹H NMR (500 MHz, DMSO-d₆) δ ppm. 8.39-8.34 (m, 2H), 7.99 (d, J=8.9 Hz, 2H), 7.95 (s, 1H), 7.27 (s, 1H), 3.74-3.66 (m, 3H), 3.25 (s, 4H), 2.64 (ddd, J=5.5, 3.6, 1.7 Hz, 5H), 2.37 (dt, J=3.7, 1.9 Hz, 1H), 2.05-1.95 (m, 3H).

### Example 121

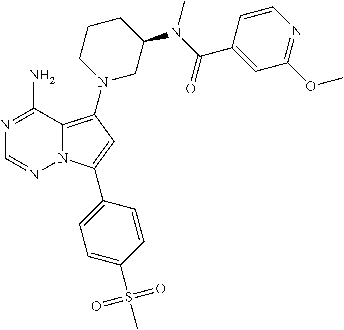 [SMILES: COc1cc(C(=O)N(C)[C@@H]2CCCN(c3cc(-c4ccc(S(C)(=O)=O)cc4)n4ncnc(N)c34)C2)ccn1]

According to the procedure for the preparation of Example 13, reaction of Example 121B and 2-methoxyisonicotinic acid afforded Example 121 (6.1 mg, 49% yield). LC-MS Method A: RT=1.565 min, [M+H]⁺=536.3; LC-MS Method B: RT=1.379 min, [M+H]⁺=536.3; ¹H NMR (500 MHz, DMSO-d₆) δ ppm 8.39-8.30 (m, 2H), 8.28-8.18 (m, 1H), 7.96 (br d, J=8.5 Hz, 2H), 7.34-7.18 (m, 1H), 6.94 (br t, J=5.0 Hz, 1H), 6.82-6.73 (m, 1H), 4.72-4.57 (m, 1H), 3.86 (s, 3H), 3.23 (s, 3H), 3.17-3.01 (m, 2H), 2.94 (br s, 3H), 1.95-1.50 (m, 4H).

### Example 122

### Preparation of (R)—N-(1-(4-amino-7-(4-(methylsulfonyl)phenyl)pyrrolo[2,1-f][1,2,4]triazin-5-yl)piperidin-3-yl)-N,5-dimethylthiophene-2-carboxamide

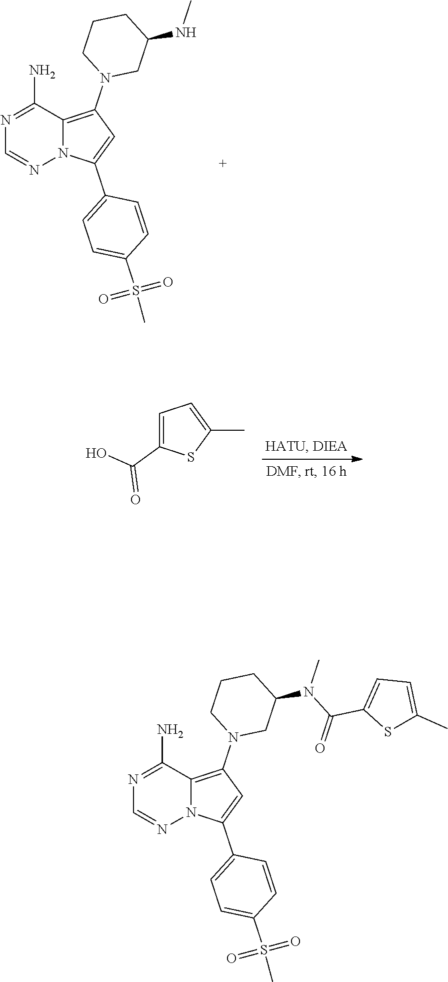

According to the procedure for the preparation of Example 13, reaction of Example 121B and 5-methylthiophene-2-carboxylic acid afforded Example 122 (6.1 mg, 49% yield). LC-MS Method A: RT=1.890 min. [M+H]⁺=525.2; LC-MS Method B: RT=1.582 min, [M+H]⁺=525.2; ¹H NMR (500 MHz, DMSO-d₆) δ ppm 8.34 (d, J=8.9 Hz, 2H), 7.95 (d, J=8.5 Hz, 2H), 7.89 (s, 1H), 7.26 (s, 2H), 6.82 (br d, J=2.7 Hz, 1H), 4.53-4.40 (m, 1H), 3.58 (br s, 1H), 3.21 (s, 3H), 3.13-2.97 (m, 4H), 2.74-2.64 (m, 1H), 2.44 (s, 3H), 1.86-1.71 (m, 5H).

### Example 123

### Preparation of (R)—N-(1-(4-amino-7-(4-(methylsulfonyl)phenyl)pyrrolo[2,1-f][1,2,4]triazin-5-yl)piperidin-3-yl)-N-methylbenzamide

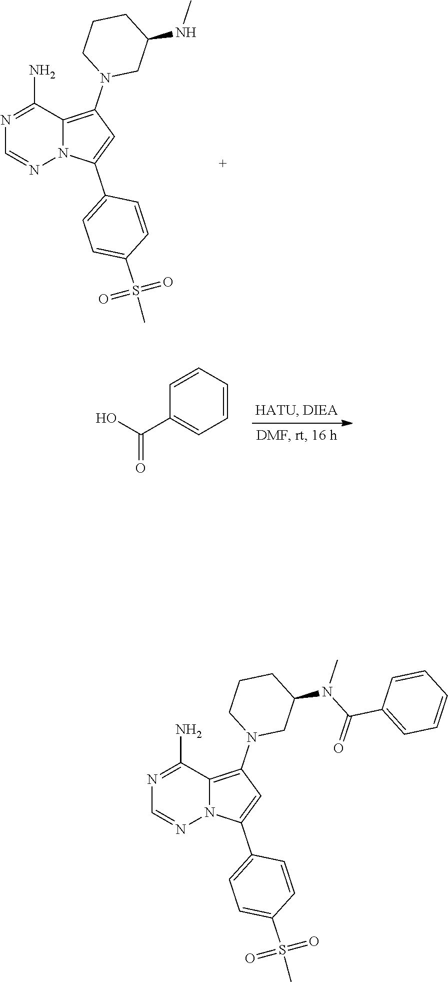 [SMILES: CN[C@@H]1CCCN(c2cc(-c3ccc(S(C)(=O)=O)cc3)n3ncnc(N)c23)C1 ; O=C(O)c1ccccc1 ; CN(C(=O)c1ccccc1)[C@@H]1CCCN(c2cc(-c3ccc(S(C)(=O)=O)cc3)n3ncnc(N)c23)C1]

According to the procedure for the preparation of Example 13, reaction of Example 121B and benzoic acid afforded Example 123 (4.6 mg, 38% yield). LC-MS Method A: RT=1.648 min, [M+H]⁺=505.2; LC-MS Method B: RT=1.469 min, [M+H]⁺=505.2; ¹H NMR (500 MHz, DMSO-d₆) δ ppm 8.33 (br dd, J=6.3, 1.4 Hz, 2H), 7.96 (br d, J=8.9 Hz, 2H), 7.89 (br s, 1H), 7.43 (br s, 3H), 7.39-7.31 (m, 2H), 3.62-3.51 (m, 1H), 3.22 (s, 3H), 3.15-2.74 (m, 6H), 2.72-2.57 (m, 1H), 2.00-1.60 (m, 4H).

### Example 124

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-methylthiophene-2-carboxamide

### Example 125

### Preparation of (R)—N-(1-(4-amino-7-(1-(methylsulfonyl-4,5-dihydro-1H-pyrrol-3-yl)pyrrolo[2,1-f][1,2,4]triazin-5-yl)piperidin-3-yl)-5-methylthiophene-2-carboxamide

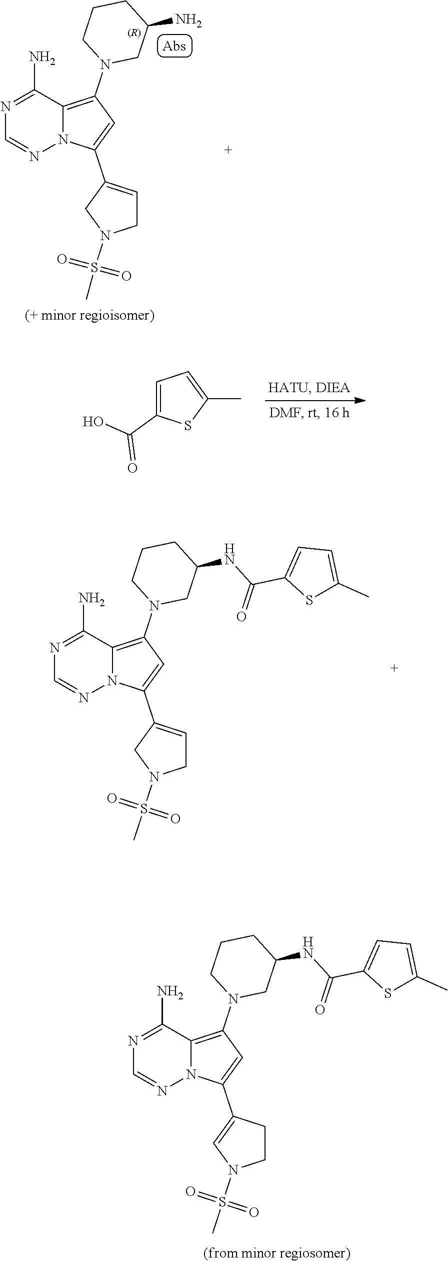

According to the procedure for the preparation of Example 112, reaction of Example 112G (+minor dihydropyrrole regioisomer) and 5-methylthiophene-2-carboxylic acid afforded the following analogs.

Example 124 (10.5 mg, 51% yield) was obtained as a white solid. MS: [M+H]⁺=502.2; HPLC Method C: RT=8.08 min; HPLC Method D: RT=5.17 min; ¹H NMR (500 MHz, DMSO-&) δ ppm 8.44-8.26 (m, 1H), 8.21 (d, J=7.8 Hz, 1H), 7.92 (s, 1H), 7.61 (d, J=3.7 Hz, 1H), 7.26-7.01 (m, 1H), 6.83 (dd, J=3.7, 1.1 Hz, 1H), 6.78 (s, 1H), 6.73 (t, J=2.1 Hz, 1H), 4.54 (td, J=4.4, 1.8 Hz, 2H), 4.30 (td, J=4.3, 2.4 Hz, 2H), 4.16-4.06 (m, 1H), 3.21-3.15 (m, 1H), 3.02 (br d, J=11.0 Hz, 1H), 2.95 (s, 3H), 2.71-2.58 (m, 2H), 2.49-2.42 (m, 3H), 1.94-1.79 (m, 3H), 1.55-1.43 (m, 1H).

Example 125 (4.0 mg, 19% yield) was obtained as a white solid. MS: [M+H]⁺=502.2; HPLC Method C: RT=8.40 min; HPLC Method D: RT=5.33 min; ¹H NMR (500 MHz, DMSO-d₆) δ ppm 8.21 (d, J=7.8 Hz, 1H), 7.97 (s, 1H), 7.61 (d, J=3.7 Hz, 1H), 7.37 (t, J=1.8 Hz, 1H), 6.83 (dd, J=3.7, 1.1 Hz, 1H), 6.62 (s, 1H), 4.18-4.07 (m, 1H), 3.79 (br t, J=9.2 Hz, 3H), 3.23-3.16 (m, 1H), 3.07 (br t, J=1.7 Hz, 2H), 3.05 (s, 3H), 3.03-2.99 (m, 1H), 2.72-2.61 (m, 2H), 2.45 (s, 3H), 1.94-1.79 (m, 3H), 1.54-1.44 (m, 1H).

### Example 126

### Preparation of N-[(3R)-1-[4-amino-7-(pyridin-4-yl)pyrrolo[2,1-f][1,2,4]triazin-5-yl]piperidin-3-yl]benzamide

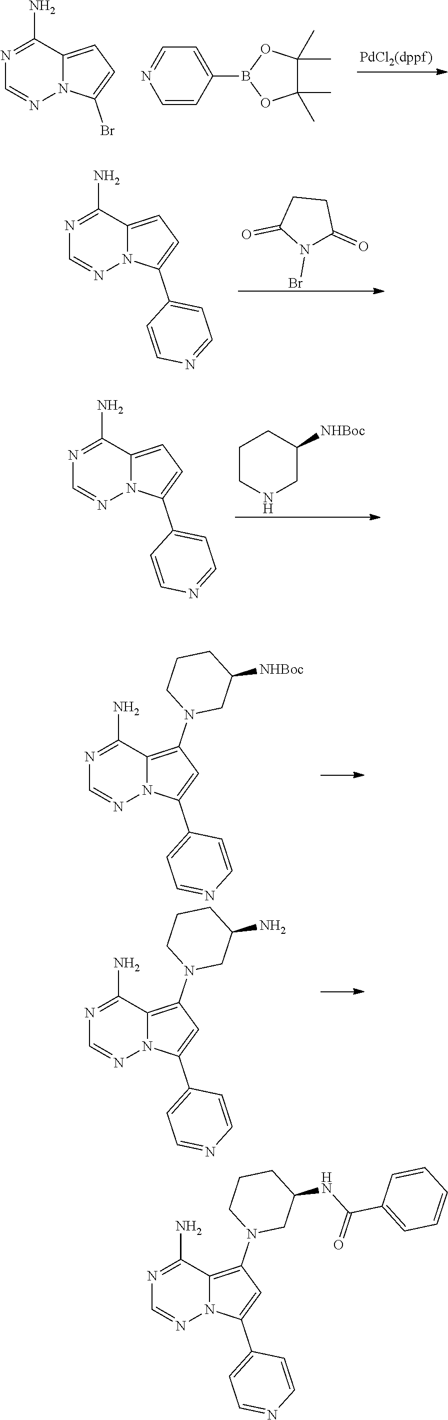

### Example 126A. 7-(pyridin-4-yl)pyrrolo[2,1-f][1,2,4]triazin-4-amine

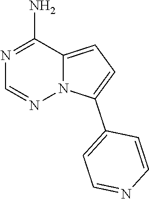 [SMILES: Nc1ncnn2c(-c3ccncc3)ccc12]

To a vial containing 7-bromopyrrolo[2,1-f][1,2,4]triazin (250 mg, 1.17 mmol), 4-(4,4,5,5-tetramethyl-1,3,2-dioxaborolan-2-yl)pyridine (481 mg, 2.35 mmol) and tripotassium phosphate (623 mg, 2.93 mmol), were added THF (10 mL) and Water (1.1 mL). The mixture was degassed (evacuated and flushed with N₂, 3×), then PdCl₂(dppf)-CH₂Cl₂ adduct (96 mg, 0.12 mmol) was added. The mixture was degassed (3×), then the vial was sealed and heated at 90° C. for 7 h. The reaction mixture was concentrated in vacuo. The crude product was purified by flash chromatography (1 to 15% methanol/methylene chloride gradient) to afford Example 126A (248 mg, 100% yield) as a yellow solid. MS: [M+H]⁺=212.2; ¹H NMR (500 MHz, DMSO-d₆) δ 8.63-8.57 (m, 2H), 8.17-8.11 (m, 2H), 8.02 (s, 1H), 7.92 (br d, J=2.6 Hz, 2H), 7.33 (d, J=4.7 Hz, 1H), 7.06 (d, J=4.7 Hz, 1H)

### Example 126B. 5-bromo-7-(pyridin-4-yl)pyrrolo[2,1-f][1,2,4]triazin-4-amine

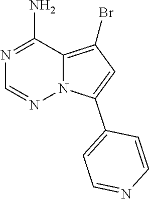 [SMILES: Nc1ncnn2c(-c3ccncc3)cc(Br)c12]

To a solution of Example 126A (100 mg, 0.473 mmol) in DMF (3 mL), was added NBS (88 mg, 0.497 mmol). The mixture was stirred at rt 45 min. The heterogeneous reaction mixture was added dropwise to a well-stirred solution of NaHCO₃ (5 g) and NaSO₂ (0.5 g) in water (100 mL). The mixture was stirred for 5 min, then was filtered and the solid collected to afford Example 126B (124 mg, 90% yield) as a tan solid. MS: [M+H]⁺=290.1; ¹H NMR (500 MHz, DMSO-d₆) δ 8.64 (d, J=5.3 Hz, 2H), 8.10 (d, J=5.3 Hz, 2H), 8.04 (s, 1H), 7.54 (s, 1H)

### Example 126C. tert-butyl (R)-(1-(4-amino-7-(pyridin-4-yl)pyrrolo[2,1-f][1,2,4]triazin-5-yl)piperidin-3-yl)carbamate TFA

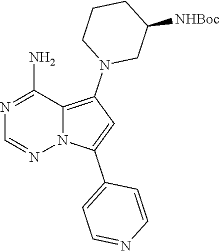 [SMILES: CC(C)(C)OC(=O)N[C@@H]1CCCN(c2cc(-c3ccncc3)n3ncnc(N)c23)C1]

Example 126B (123 mg, 0.424 mmol), Ir[dF(CF₃)ppy]₂(dtbbpy)PF₆ (9.5 mg, 8.5 μmol), tert-butyl (R)-piperidin-3-ylcarbamate (340 mg, 1.70 mmol) and DABCO (171 mg, 1.53 mmol) were placed in a pressure relief vial. The reaction mixture was degassed (3× vacuum/nitrogen), then a solution of NiBr₂-DME (26.2 mg, 0.085 mmol) in DMA (6 mL) was added. The reaction mixture was degassed again, capped and stirred under blue LED irradiation with fan cooling at rt for 10 days. The reaction mixture was diluted with EtOAc (200 mL), washed with water (50 mL) and brine (2×50 mL). The organic phase was filtered, rinsing the collected solid extensively with EtOAc. The combined organic phase was concentrated. The residue was suspended in CHCl3 (20 mL), then was filtered. The filtrate was concentrated, then was purified by flash chromatography (1 to 20% methanol/methylene chloride gradient). The product was further purified by prep HPLC (CH₃CN/H₂O/TFA) to afford Example 126C (71 mg, 32% yield) as a yellow solid. MS: [M+H]⁺=410.3; ¹H NMR (500 MHz, DMSO-d₆) δ 8.78 (d, J=6.7 Hz, 2H), 8.66 (br d, J=6.4 Hz, 2H), 8.41 (br s, 1H), 8.09 (s, 1H), 7.64 (s, 1H), 7.20 (br s, 1H), 7.14-7.13 (m, 1H), 7.10 (br s, 1H), 3.71 (br s, 2H), 3.17-3.06 (m, 1H), 2.91 (br s, 1H), 1.94-1.64 (m, 3H), 1.38 (s, 9H)

### Example 126D. (R)-5-(3-aminopiperidin-1-yl)-7-(pyridin-4-yl)pyrrolo[2,1-f][1,2,4]triazin-4-amine, 2 HCl

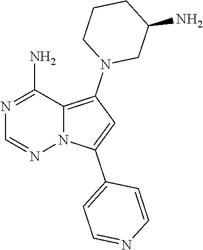 [SMILES: Nc1ncnn2c(-c3ccncc3)cc(N3CCC[C@@H](N)C3)c12]

Example 126C (70 mg, 0.13 mmol) was suspended in 4N HCl in dioxane (5 mL, 20 mmol). The mixture was stirred at rt for 1.25 h. The suspension was concentrated to afford Example 126D (51 mg, 100% yield) as an orange solid. LC-MS: [M+H]⁺=310.3

### Example 126

To a vial containing benzoic acid (4.8 mg, 0.039 mmol) and HATU (14.9 mg, 0.039 mmol), was added a solution of Example 126D (12.5 mg, 0.033 mmol) and DIEA (0.029 mL, 0.163 mmol) in DMF (1 mL). The mixture was stirred at rt for 19.5 h. The reaction was quenched with MeOH (0.1 mL), followed by TFA (2 drops), then was purified by preparative HPLC to afford Example 126 (7.0 mg, 52% yield). LC-MS Method A: RT=1.64 min, [M+H]⁺=413.84; LC-MS Method B: RT=1.14 min, [M+H]⁺=414.15; ¹H NMR (500 MHz, DMSO-d₆) δ 8.59 (br d, J=5.3 Hz, 2H), 8.38 (br d, J=7.6 Hz, 1H), 8.14 (d, J=6.2 Hz, 1H), 8.12 (br s, 1H), 7.93 (s, 1H), 7.84 (d, J=7.2 Hz, 2H), 7.55-7.49 (m, 1H), 7.49-7.41 (m, 2H), 7.31 (s, 1H), 7.04 (br s, 1H), 4.28-4.12 (m, 1H), 3.30-3.18 (m, 1H), 3.03 (br s, 1H), 2.73 (br s, 1H), 1.96-1.86 (m, 2H), 1.86-1.74 (m, 1H), 1.64-1.45 (m, 1H)

### Example 127

### N-[(3R)-1-[4-amino-7-(pyridin-4-yl)pyrrolo[2,1-f][1,2,4]triazin-5-yl]piperidin-3-yl]-2-methoxypyridine-4-carboxamide

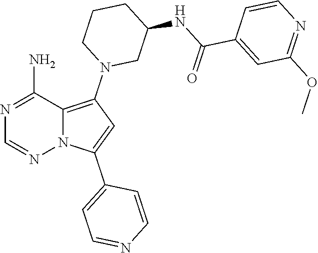 [SMILES: COc1cc(C(=O)N[C@@H]2CCCN(c3cc(-c4ccncc4)n4ncnc(N)c34)C2)ccn1]

According to the procedure for the preparation of Example 126, coupling of Example 126C with 2-methoxyisonicotinic acid, afforded Example 127 (7.2 mg, 50% yield). LC-MS Method A: RT=1.51 min, [M+H]⁺=445.19; LC-MS Method B: RT=1.09 min, [M+H]⁺=445.19; ¹H NMR (500 MHz, DMSO-d₆) δ 8.61 (br d, J=7.3 Hz, 1H), 8.58 (br d, J=5.4 Hz, 2H), 8.27 (d, J=5.2 Hz, 1H), 8.12 (d, J=6.1 Hz, 2H), 8.06 (br s, 1H), 7.92 (s, 1H), 7.32 (dd, J=5.3, 1.2 Hz, 1H), 7.29 (s, 1H), 7.17 (s, 1H), 7.08-6.94 (m, 1H), 4.18 (br d, J=7.5 Hz, 1H), 3.88 (s, 2H), 3.26-3.15 (m, 1H), 3.08-2.95 (m, 1H), 2.83-2.67 (m, 1H), 1.97-1.84 (m, 2H), 1.84-1.73 (m, 1H), 1.62-1.44 (m, 1H)

### Example 128

### N-[(3R)-1-[4-amino-7-(pyridin-4-yl)pyrrolo[2,1-f][1,2,4]triazin-5-yl]piperidin-3-yl]-5-methylthiophene-2-carboxamide

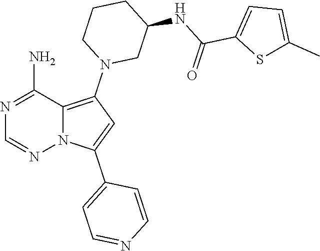 [SMILES: Cc1ccc(C(=O)N[C@@H]2CCCN(c3cc(-c4ccncc4)n4ncnc(N)c34)C2)s1]

According to the procedure for the preparation of Example 126, coupling of Example 126C with 5-methylthiophene-2-carboxylic acid, afforded Example 128 (8.3 mg, 59% yield). LC-MS Method A: RT=1.72 min, [M+H]⁺=434.30; LC-MS Method B: RT=1.21 min, [M+H]⁺=434.14 ¹H NMR (500 MHz, DMSO-d₆) δ 8.59 (br d, J=5.4 Hz, 2H), 8.24 (br d, J=7.8 Hz, 1H), 8.14 (d, J=6.1 Hz, 2H), 8.08 (br s, 1H), 7.93 (s, 1H), 7.61 (d, J=3.5 Hz, 1H), 7.31 (s, 1H), 7.00 (br s, 1H), 6.82 (d, J=2.7 Hz, 1H), 4.11 (br dd, J=4.3, 1.2 Hz, 1H), 3.27-3.15 (m, 1H), 3.09-2.98 (m, 1H), 2.79-2.62 (m, 1H), 2.44 (s, 3H), 1.96-1.72 (m, 3H), 1.60-1.44 (m, 1H)

### Example 129

### N-[(3R)-1-[4-amino-7-(pyridin-4-yl)pyrrolo[2,1-f][1,2,4]triazin-5-yl]piperidin-3-yl]-5-chlorothiophene-2-carboxamide

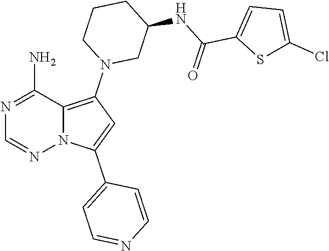 [SMILES: Nc1ncnn2c(-c3ccncc3)cc(N3CCC[C@@H](NC(=O)c4ccc(Cl)s4)C3)c12]

According to the procedure for the preparation of Example 126, coupling of Example 126C with 5-chlorothiophene-2-carboxylic acid, afforded Example 129 (7.6 mg, 51% yield). LC-MS Method A: RT=1.76 min, [M+H]⁺=454.08; LC-MS Method B: RT=1.30 min, [M+H]⁺=454.09; ¹H NMR (500 MHz, DMSO-d₆) δ 8.58 (br d, J=5.8 Hz, 2H), 8.48 (d, J=7.7 Hz, 1H), 8.16-8.10 (m, 2H), 8.06 (br s, 1H), 7.92 (s, 1H), 7.70 (d, J=4.0 Hz, 1H), 7.30 (s, 1H), 7.17 (d, J=4.1 Hz, 1H), 7.00 (br s, 1H), 4.21-4.00 (m, 1H), 3.26-3.15 (m, 1H), 3.04 (br dd, J=11.1, 3.2 Hz, 1H), 2.81-2.64 (m, 1H), 1.98-1.74 (m, 3H), 1.60-1.42 (m, 1H)

### Example 130

### Preparation of N-[(3R)-1-[4-amino-7-(2-fluoropyridin-4-yl)pyrrolo[2,1-f][1,2,4]triazin-5-yl]piperidin-3-yl]-5-chlorothiophene-2-carboxamide

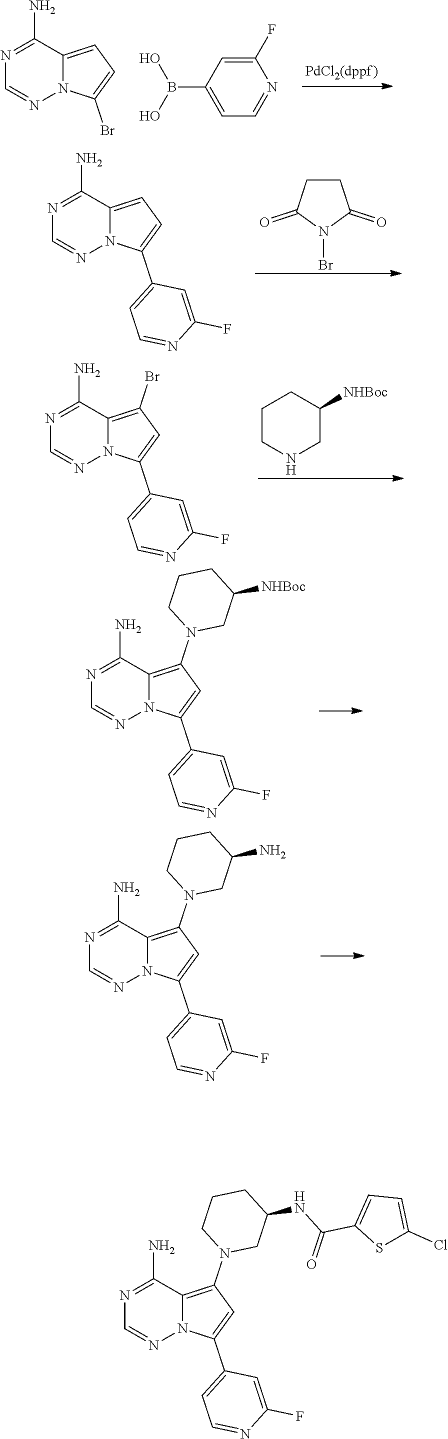

### Example 130A. 7-(2-fluoropyridin-4-yl)pyrrolo[2,1-f][1,2,4]triazin-4-amine

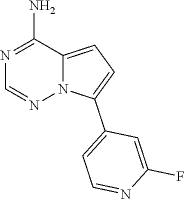 [SMILES: Nc1ncnn2c(-c3ccnc(F)c3)ccc12]

To a vial containing 7-bromopyrrolo-[2,1-f][1,2,4]triazin-4-amine (250 mg, 1.17 mmol), (2-fluoropyridin-4-yl)boronic acid (275 mg, 1.95 mmol) and tripotassium phosphate (623 mg, 2.93 mmol), were added THF (10 mL) and Water (1.11 mL). The mixture was degassed (evacuated and flushed with N₂, 3×), then PdCl₂(dppf)-CH₂Cl₂ adduct (96 mg, 0.12 mmol) was added. The mixture was degassed (3×), then the vial was sealed and heated at 90° C. for 5.25 h. The reaction mixture was concentrated, then was purified by flash chromatography (gradient from 1 to 15% methanol/methylene chloride). The product was suspended in DCM (~3 mL). The solid was collected by filtration to afford Example 130A (215 mg, 80% yield) as a beige solid. MS: [M+H]⁺=230.2; ¹H NMR (500 MHz, DMSO-d₆) δ 8.27 (d, J=5.5 Hz, 1H), 8.12-8.09 (m, 1H), 8.07 (s, 1H), 8.01 (s, 1H), 7.99 (br s, 2H), 7.45 (d, J=4.7 Hz, 1H), 7.07 (d, J=4.7 Hz, 1H)

### Example 130B. 5-bromo-7-(2-fluoropyridin-4-yl)pyrrolo[2,1-f][1,2,4]triazin-4-amine

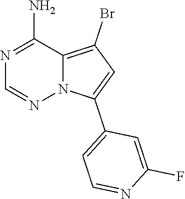 [SMILES: Nc1ncnn2c(-c3ccnc(F)c3)cc(Br)c12]

To a solution of Example 130A (213 mg, 0.929 mmol) in DMF (6 mL), was added NBS (165 mg, 0.929 mmol). The mixture was stirred at rt protected from light for 1.5 h. Additional NBS (33 mg, 0.19 mmol) was added and the mixture was stirred at rt for 1.5 h. The heterogeneous reaction mixture was added dropwise to a well-stirred solution of NaHCO₃ (5 g) and NaSO₂ (0.5 g) in water (100 mL). The mixture was stirred for 10 min, then was filtered and the solid collected. The solid was rinsed with H₂O and sucked dry to afford Example 130B (290 mg, 100% yield) as an off-white solid. MS: [M+H]⁺=308.1; ¹H NMR (500 MHz, DMSO-d₆) δ 8.30 (d, J=5.2 Hz, 1H), 8.10 (t, J=1.7 Hz, 1H), 8.09 (s, 1H), 7.96 (s, 1H), 7.67 (s, 1H)

### Example 130C. tert-butyl (R)-(1-(4-amino-7-(2-fluoropyridin-4-yl)pyrrolo[2,1-f][1,2,4]triazin-5-yl)piperidin-3-yl)carbamate, 2 TFA

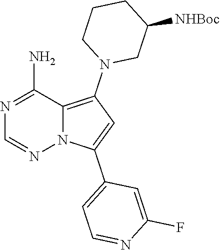 [SMILES: CC(C)(C)OC(=O)N[C@@H]1CCCN(c2cc(-c3ccnc(F)c3)n3ncnc(N)c23)C1]

Example 130B (285 mg, 0.925 mmol), Ir[dF(CF₃)ppy]₂(dtbbpy)PF₆ (20.8 mg, 0.018 mmol), tert-butyl (R)-piperidin-3-ylcarbamate (741 mg, 3.70 mmol) and DABCO (374 mg, 3.33 mmol) were placed in a 40 mL pressure relief vial. The reaction mixture was degassed (3× vacuum/nitrogen), then a solution of NiBr₂-DME (57.1 mg, 0.185 mmol) in DMA (16 mL) was added. The reaction mixture was degassed again (3×), capped and stirred under blue LED irradiation with fan cooling at rt for 10 days. The mixture was diluted with H₂O (300 mL) and EtOAc (200 mL). A portion of material remained insoluble and was removed by filtration. The phases were separated, then the aqueous phase was extracted with EtOAc (2×). The combined organic phase was washed with H₂O and brine, dried (Na₂SO₄) and concentrated. The residue was suspended in CHCl3 (20 mL) and filtered, removing insoluble starting material. The filtrate was concentrated, then was purified by flash chromatography (0 to 100% ethyl acetate/hexanes gradient). The product was further purified by preparative HPLC (CH₃CN/H₂O/TFA) to afford Example 130C (85 mg, 14% yield) as a pale yellow solid. MS: [M+H]⁺=428.3; ¹H NMR (500 MHz, Methanol-d₄) δ 8.27 (d, J=5.5 Hz, 1H), 8.00 (s, 1H), 7.97 (dt, J=5.4, 1.6 Hz, 1H), 7.88 (s, 1H), 7.35 (s, 1H), 3.84-3.73 (m, 1H), 3.11 (br d, J=11.0 Hz, 1H), 2.90 (br s, 1H), 2.72 (br d, J=6.3 Hz, 1H), 1.98-1.89 (m, 2H), 1.89-1.81 (m, 1H), 1.45 (s, 9H); ¹⁹F NMR (471 MHz, METHANOL-d₄) δ −70.52 (s), −77.31 (s)

### Example 130D. (R)-5-(3-aminopiperidin-1-yl)-7-(2-fluoropyridin-4-yl)pyrrolo[2,1-f][1,2,4]triazin-4-amine, HCl

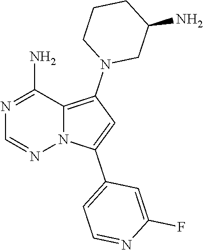 [SMILES: Nc1ncnn2c(-c3ccnc(F)c3)cc(N3CCC[C@@H](N)C3)c12]

Example 130C (83 mg, 0.127 mmol) was suspended in 4N HCl in dioxane (1 mL). The mixture was stirred at rt for 2.5 h. The mixture was concentrated, then was coevaporated with EtOAc to afford Example 130D (74 mg) as a yellow solid, which was used without further purification. MS: [M+H]⁺=328.2

### Example 130

To a solution of Example 130D (13.5 mg, 0.031 mmol), 5-chlorothiophene-2-carboxylic acid (6.0 mg, 0.037 mmol) and DIEA (0.032 mL, 0.19 mmol) in DMF (1 mL), was added HATU (14.1 mg, 0.037 mmol). The mixture was stirred at rt for 19 h. The reaction was quenched with MeOH (0.1 mL), followed by TFA (2 drops), then was purified by preparative HPLC to afford Example 130 (11.2 mg, 73% yield). LC-MS Method A: RT=1.96 min, [M+H]⁺=472.08; LC-MS Method B: RT=1.63 min, [M+H]⁺=472.08; ¹H NMR (500 MHz, DMSO-d₆) δ 8.49 (br d, J=7.6 Hz, 1H), 8.23 (d, J=5.5 Hz, 1H), 8.17-8.07 (m, 1H), 8.06 (br d, J=4.6 Hz, 1H), 7.95 (s, 2H), 7.69 (br d, J=3.7 Hz, 1H), 7.39 (s, 1H), 7.16 (d, J=4.3 Hz, 1H), 7.11-6.93 (m, 1H), 4.18-4.03 (m, 1H), 3.27-3.12 (m, 1H), 3.09-2.99 (m, 1H), 2.77-2.61 (m, 1H), 1.97-1.72 (m, 3H), 1.61-1.40 (m, 1H)

### Example 131

### N-[(3R)-1-[4-amino-7-(2-fluoropyridin-4-yl)pyrrolo[2,1-f][1,2,4]triazin-5-yl]piperidin-3-yl]-5-methylthiophene-2-carboxamide

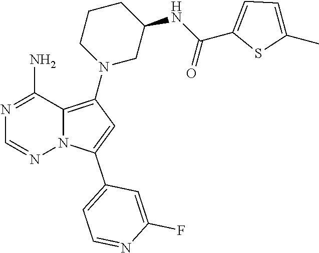 [SMILES: Cc1ccc(C(=O)N[C@@H]2CCCN(c3cc(-c4ccnc(F)c4)n4ncnc(N)c34)C2)s1]

According to the procedure for the preparation of Example 130, coupling of Example 130D with 5-methylthiophene-2-carboxylic acid afforded Example 131. LC-MS Method A: RT=1.85 min, [M+H]⁺=452.14; LC-MS Method B: RT=1.54 min, [M+H]⁺=452.12; ¹H NMR (500 MHz, DMSO-d₆) δ 8.26 (br d, J=7.6 Hz, 1H), 8.23 (d, J=5.5 Hz, 1H), 8.08 (br s, 1H), 8.06 (br d, J=5.2 Hz, 1H), 7.95 (s, 2H), 7.59 (br d, J=3.4 Hz, 1H), 7.39 (s, 1H), 7.02 (br s, 1H), 6.82 (br d, J=2.7 Hz, 1H), 4.09 (br s, 1H), 3.24-3.14 (m, 1H), 3.08-2.99 (m, 1H), 2.77-2.61 (m, 2H), 2.43 (s, 3H), 1.95-1.71 (m, 3H), 1.56-1.45 (m, 1H)

### Example 132

### Preparation of N-[(3R)-1-[4-amino-7-(2-methylpyridin-4-yl)pyrrolo[2,1-f][1,2,4]triazin-5-yl]piperidin-3-yl)-5-chlorothiophene-2-carboxamide

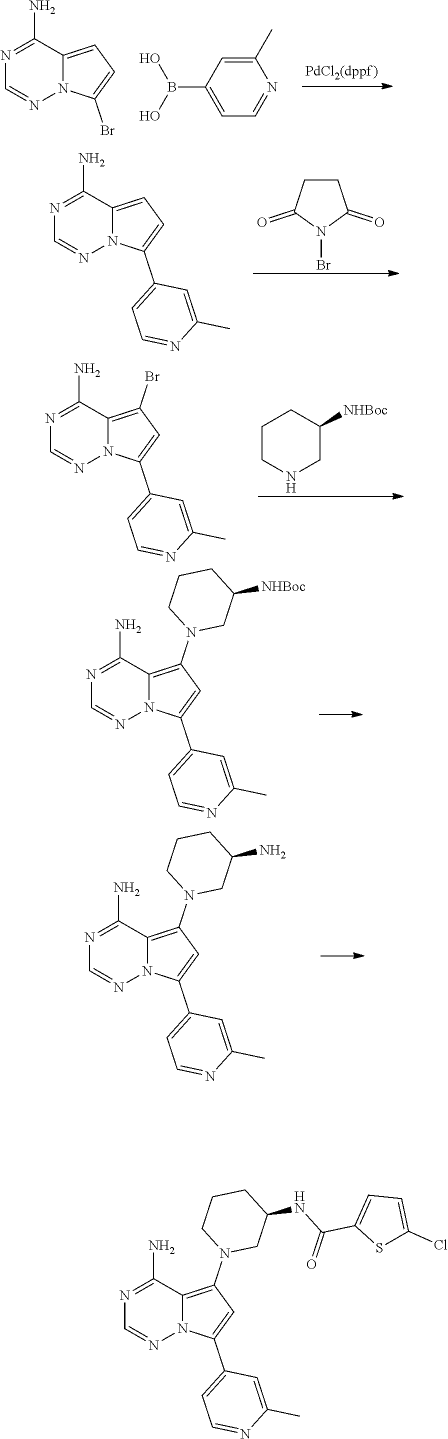

### Example 132A. 7-(2-methylpyridin-4-yl)pyrrolo 2,1-f][1,2,4]triazin-4-amine

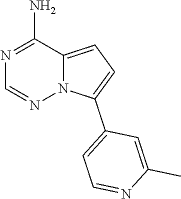 [SMILES: Cc1cc(-c2ccc3c(N)ncnn23)ccn1]

To a vial containing 7-bromopyrrolo[2,1-][1,2,4]triazin-4-amine (250 mg, 1.17 mmol), (2-methylpyridin-4-yl)boronic acid (321 mg, 2.35 mmol) and tripotassium phosphate (623 mg, 2.93 mmol), were added THF (10 mL) and Water (1.11 mL). The mixture was degassed (evacuated and flushed with N₂, 3×), then PdCl₂(dppf)-CH₂Cl₂ adduct (96 mg, 0.117 mmol) was added. The mixture was degassed (3×), then the vial was sealed and heated at 90° C. for 5.25 h. The reaction mixture was concentrated. The crude product was purified by flash chromatography (solid loaded from Celite, 1 to 15% methanol/methylene chloride gradient). The product was suspended in DCM (~3 mL). The suspension was filtered and the solid was collected, rinsed with DCM (2×1 mL), and sucked dry to afford Example 132A (212 mg, 80% yield) as a beige solid. MS: [M+H]⁺=230.2; ¹H NMR (500 MHz, DMSO-d₆) δ 8.27 (d, J=5.5 Hz, 1H), 8.12-8.09 (m, 1H), 8.07 (s, 1H), 8.01 (s, 1H), 7.99 (br s, 2H), 7.45 (d, J=4.7 Hz, 1H), 7.07 (d, J=4.7 Hz, 1H)

### Example 132B. 5-bromo-7-(2-methylpyridin-4-yl)pyrrolo[2,1-f][1,2,4]triazin-4-amine

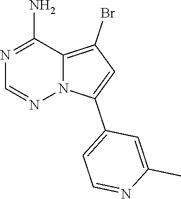 [SMILES: Cc1cc(-c2cc(Br)c3c(N)ncnn23)ccn1]

To a solution of Example 132A (210 mg, 0.932 mmol) in DMF (5 mL) at rt, was added NBS (174 mg, 0.979 mmol). The mixture was stirred at rt for 15 min. The mixture was added dropwise to a well-stirred solution of NaHCO₃ (5 g) and Na₂SO₃ (0.5 g) in 100 mL H₂O. The mixture was stirred for 15 min. The resultant aqueous suspension was filtered and the solid collected. The solid was rinsed with H₂O, followed by Et₂₀. The residue was sucked dry, then was dried in vacuo to afford Example 132B (259 mg, 91% yield) as an off-white solid.

MS: [M+H]⁺=304.1; ¹H NMR (500 MHz, DMSO-d₅) δ 8.50 (d, J=5.0 Hz, 1H), 8.04 (s, 1H), 7.96 (d, J=0.6 Hz, 1H), 7.90 (dd, J=5.3, 1.3 Hz, 1H), 7.50 (s, 1H), 2.51 (s, 3H)

### Example 132C. tert-butyl (R)-(1-(4-amino-7-(2-methylpyridin-4-yl)pyrrolo[2,1-f][1,2,4]triazin-5-yl)piperidin-3-yl)carbamate

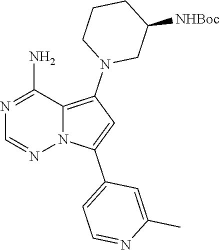 [SMILES: Cc1cc(-c2cc(N3CCC[C@@H](NC(=O)OC(C)(C)C)C3)c3c(N)ncnn23)ccn1]

Example 132B (258 mg, 0.848 mmol), Ir[dF(CF₃)ppy]₂(dtbbpy)PF₆ (19.0 mg, 0.017 mmol), tert-butyl (R)-piperidin-3-ylcarbamate (680 mg, 3.39 mmol) and DABCO (343 mg, 3.05 mmol) were placed in a 40 mL pressure relief vial. The reaction mixture was degassed (3× vacuum/nitrogen), then a solution of NiBr₂-DME (52.4 mg, 0.170 mmol) in DMA (14.1 mL) was added. The reaction mixture was degassed again (3×), capped and stirred under blue LED irradiation with fan cooling at rt for 11 days. The reaction mixture was partitioned with EtOAc (200 mL) and water (300 mL). The aqueous phase was extracted with EtOAc (2×100 mL). The combined organic extract was filtered, then the filtrate was washed with brine, dried (Na₂SO₄) and concentrated. The crude product was purified by flash chromatography (2 to 20% MeOH/DCM gradient). The residue was suspended in DCM (5 mL), then filtered. The filtrate was concentrated to afford Example 132C (109 mg, 30% yield) as a pale yellow solid. MS: [M+H]⁺=424.3; ¹H NMR (500 MHz, Methanol-d₄) δ 8.39 (d, J=5.5 Hz, 1H), 8.06 (s, 1H), 7.97 (dd, J=5.5, 1.4 Hz, 1H), 7.84 (s, 1H), 7.18 (s, 1H), 3.74 (br s, 1H), 3.27-3.18 (m, 1H), 2.87-2.73 (m, 1H), 2.57 (s, 3H), 1.98-1.86 (m, 2H), 1.80 (br d, J=8.9 Hz, 1H), 1.45 (s, 9H)

### Example 132D. (R)-5-(3-aminopiperidin-1-yl)-7-(2-methylpyridin-4-yl)pyrrolo[2,1-f][1,2,4]triazin-4-amine, 3 HCl

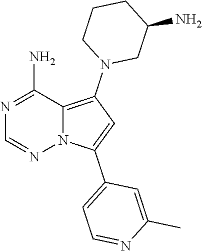 [SMILES: Cc1cc(-c2cc(N3CCC[C@@H](N)C3)c3c(N)ncnn23)ccn1]

Example 132C (109 mg, 0.257 mmol) was suspended in 4N HCl in dioxane (5 mL). The mixture was stirred at rt for 3 h, then was concentrated to afford Example 132D (120 mg) as a yellow solid, which was used without additional purification. MS: [M+H]⁺=324.3; ¹H NMR (500 MHz, Methanol-d₄) δ 8.72-8.64 (m, 3H), 8.14 (s, 1H), 7.75 (s, 1H), 3.72-3.67 (m, 1H), 3.49-3.42 (m, 2H), 2.83 (s, 3H), 2.19-2.10 (m, 1H), 2.07-2.00 (m, 1H), 1.97-1.88 (m, 1H), 1.78-1.64 (m, 1H)

### Example 132

To a solution of Example 132D (15 mg, 0.035 mmol), 5-chlorothiophene-2-carboxylic acid (6.8 mg, 0.042 mmol) and DIEA (0.036 mL, 0.21 mmol) in DMF (1 mL), was added HATU (15.8 mg, 0.042 mmol). The mixture was stirred at rt for 15.5 h. The reaction was quenched with MeOH (0.1 mL), followed by TFA (2 drops), then was purified by preparative HPLC to afford Example 132 (4.4 mg, 26% yield). LC-MS Method A: RT=RT=1.76 min, [M+H]⁺=468.08; LC-MS Method B: RT=1.34 min, [M+H]⁺=468.08; ¹H NMR (500 MHz, DMSO-d₆) δ 8.49 (br d, J=7.3 Hz, 1H), 8.43 (br d, J=4.9 Hz, 1H), 7.99-7.92 (m, 2H), 7.91 (s, 1H), 7.69 (br d, J=3.7 Hz, 1H), 7.25 (s, 1H), 7.16 (d, J=4.0 Hz, 1H), 4.10 (br d, J=4.3 Hz, 1H), 3.25-3.12 (m, 1H), 3.08-2.97 (m, 1H), 2.95-2.86 (m, 1H), 2.81-2.61 (m, 2H), 2.49 (br s, 3H), 1.96-1.85 (m, 2H), 1.84-1.72 (m, 1H), 1.59-1.43 (m, 1H), 1.15 (br t, J=7.3 Hz, 1H)

### Example 133 N-[(3R)-1-[4-amino-7-(2-methylpyridin-4-yl)pyrrolo[2,1-f][1,2,4]triazin-5-yl]piperidin-3-yl]-5-methylthiophene-2-carboxamide

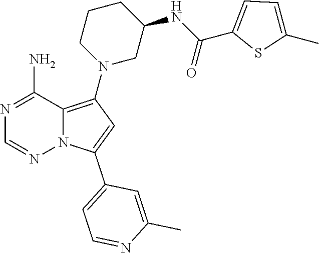 [SMILES: Cc1cc(-c2cc(N3CCC[C@@H](NC(=O)c4ccc(C)s4)C3)c3c(N)ncnn23)ccn1]

According to the procedure for the preparation of Example 132, coupling of Example 132D with 5-methylthiophene-2-carboxylic acid afforded Example 133. LC-MS Method A: RT=1.72 min, [M+H]⁺=448.14; LC-MS Method B: RT=1.26 min, [M+H]⁺=448.17; ¹H NMR (500 MHz, DMSO-d₆) δ 8.60 (br d, J=6.1 Hz, 1H), 8.46 (br d, J=5.8 Hz, 1H), 8.41 (br s, 1H), 8.26 (br d, J=7.3 Hz, 1H), 8.06-8.02 (m, 1H), 7.61 (br d, J=3.4 Hz, 1H), 7.54 (s, 1H), 6.83 (br d, J=2.1 Hz, 2H), 4.19-4.04 (m, 1H), 3.27-3.12 (m, 1H), 2.63 (s, 3H), 2.33 (s, 3H), 1.98-1.77 (m, 4H), 1.53 (br dd, J=14.3, 8.9 Hz, 2H), 1.16 (br t, J=7.3 Hz, 1H)

### Example 134

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chlorothiophene-2-carboxamide

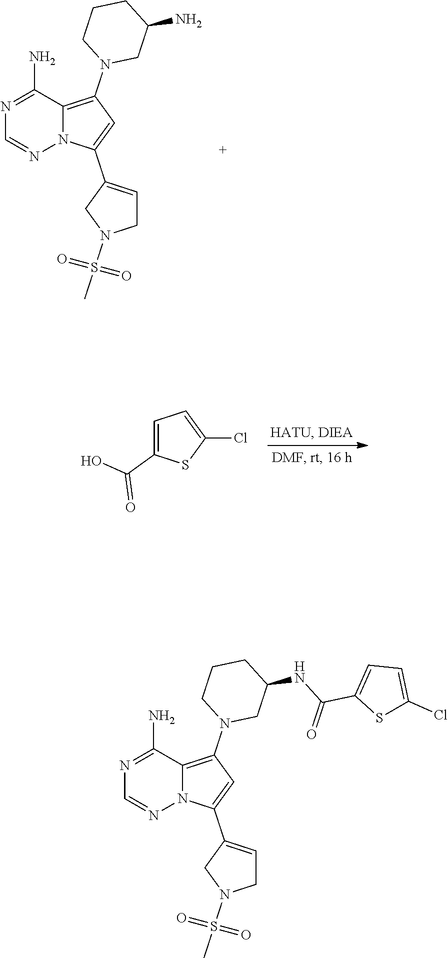 [SMILES: CS(=O)(=O)N1CC=C(c2cc(N3CCC[C@@H](N)C3)c3c(N)ncnn23)C1 ; O=C(O)c1ccc(Cl)s1 ; CS(=O)(=O)N1CC=C(c2cc(N3CCC[C@@H](NC(=O)c4ccc(Cl)s4)C3)c3c(N)ncnn23)C1]

According to the procedure for the preparation of Example 112, reaction of Example 112G and 5-chlorothiophene-2-carboxylic acid afforded Example 134 (7.0 mg, 39% yield). LC-MS Method A: RT=1.759 min, [M+H]⁺=522.1; LC-MS Method B: RT=1.498 min, [M+H]⁺=522.1; ¹H NMR (500 MHz, DMSO-d₆) δ ppm 8.46 (br d, J=7.8 Hz, 1H), 7.86 (s, 1H), 7.69 (d, J=4.1 Hz, 1H), 7.16 (d, J=4.1 Hz, 1H), 6.76-6.71 (m, 1H), 6.72-6.68 (m, 1H), 4.56-4.47 (m, 2H), 4.33-4.23 (m, 2H), 4.17-4.01 (m, 1H), 3.54-3.42 (m, 4H), 3.19-3.10 (m, 1H), 3.05-2.98 (m, 1H), 2.94 (s, 3H), 2.72-2.58 (m, 2H), 1.96-1.73 (m, 3H), 1.56-1.42 (m, 1H).

### Example 135

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)thiophene-2-carboxamide, TFA

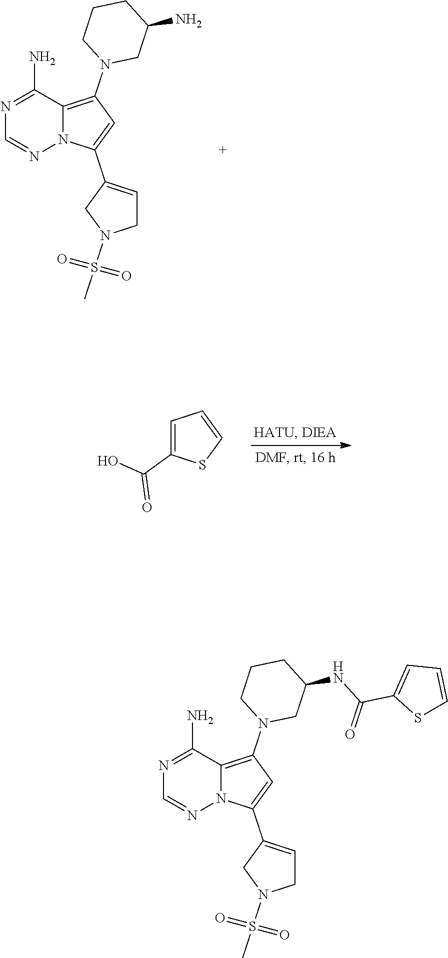 [SMILES: CS(=O)(=O)N1CC=C(c2cc(N3CCC[C@@H](N)C3)c3c(N)ncnn23)C1 ; O=C(O)c1cccs1 ; CS(=O)(=O)N1CC=C(c2cc(N3CCC[C@@H](NC(=O)c4cccs4)C3)c3c(N)ncnn23)C1]

According to the procedure for the preparation of Example 112, reaction of Example 112G and thiophene-2-carboxylic acid afforded Example 135 (14.3 mg, 58% yield). LC-MS Method A: RT=1.581 min, [M+H]⁺=488.1; LC-MS Method B: RT=1.330 min, [M+H]⁺=488.1; ¹H NMR (500 MHz, DMSO-d₆) δ ppm 8.45-8.33 (m, 1H), 7.88 (br d, J=17.1 Hz, 1H), 7.82-7.75 (m, 1H), 7.75-7.66 (m, 1H), 7.22-7.06 (m, 1H), 6.84-6.63 (m, 1H), 4.57-4.47 (m, 1H), 4.34-4.24 (m, 1H), 4.22-4.03 (m, 1H), 3.71-3.55 (m, 2H), 3.54-3.41 (m, 1H), 3.30-3.09 (m, 1H), 3.09-2.79 (m, 4H), 2.74-2.59 (m, 1H), 2.00-1.74 (m, 3H), 1.57-1.44 (m, 1H), 1.15 (br t, J=7.3 Hz, 1H).

### Example 136

### Preparation of (R)—N-(1-(4-amino-7-(4-(methylsulfonyl)phenyl)pyrrolo[2,1-f][1,2,4]triazin-5-yl)pyrrolidin-3-yl)-5-methylthiophene-2-carboxamide, TFA

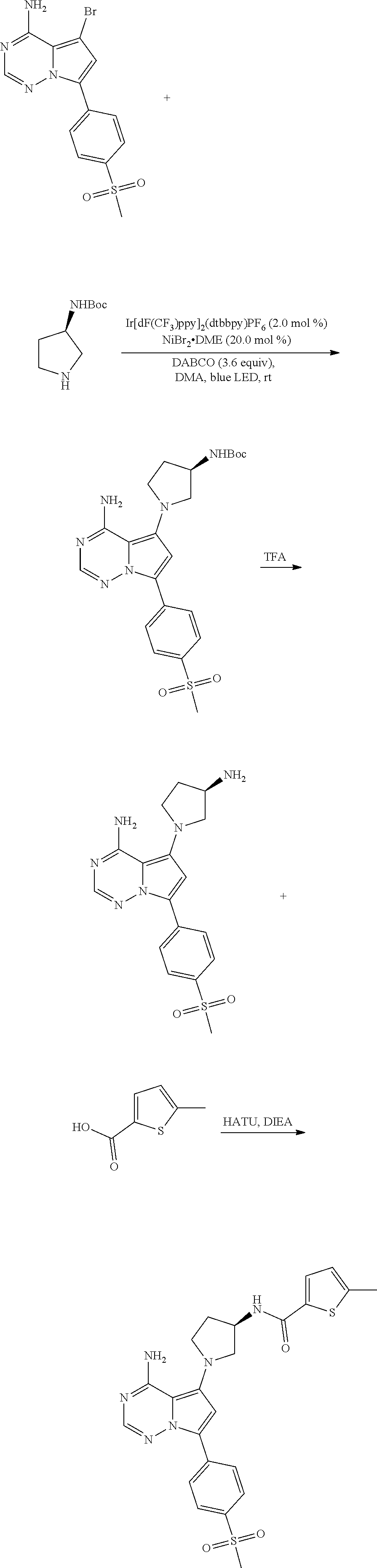

### Example 136A. tert-Butyl (R)-(1-(4-amino-7-(4-(methylsulfonyl)phenyl)pyrrolo[2,1-f][1,2,4]triazin-5-yl)pyrrolidin-3-yl)carbamate

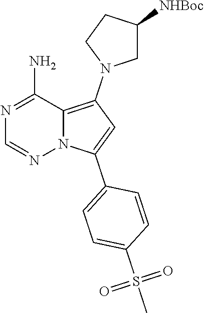 [SMILES: CC(C)(C)OC(=O)N[C@@H]1CCN(c2cc(-c3ccc(S(C)(=O)=O)cc3)n3ncnc(N)c23)C1]

Example 1B (110 mg, 0.30 mmol), Ir[dF(CF₃)ppy]₂(dtbbpy)PF₆ (6.7 mg, 6.0 μmol), tert-butyl (R)-pyrrolidin-3-ylcarbamate (223 mg, 1.20 mmol), sodium trifluoroacetate (147 mg, 1.08 mmol) and DABCO (121 mg, 1.08 mmol) were placed in a pressure relief vial. The reaction mixture was degassed (3× vacuum/nitrogen), then a solution of NiBr₂-DME (18.5 mg, 0.060 mmol) in DMA (5.0 mL) was added. The reaction mixture was degassed again, capped and stirred under blue LED irradiation with fan cooling at rt for 3 d. The crude reaction mixture was diluted with EtOAc, washed with water (3×), brine, dried (Na₂SO₄) and filtered. EtOAc was removed under reduced pressure, the residue was dissolved in DCM, and was purified by flash chromatography (20-100% EtOAc/DCM gradient). Fractions were combined, concentrated under reduced pressure and the residue was further purified by preparative HPLC to give Example 136A (0.030 g, 21% yield) as an off-white solid. MS: [M+H]⁺=473.3; ¹H NMR (500 MHz, DMSO-d₆) δ ppm 8.35-8.31 (m, 2H), 7.98 (d, J=8.5 Hz, 2H), 7.95-7.90 (m, 1H), 7.35 (br d, J=7.3 Hz, 1H), 7.17 (s, 1H), 4.26-4.12 (m, 2H), 4.02-3.88 (m, 2H), 3.27 (br dd, J=8.3, 3.7 Hz, 1H), 2.95-2.88 (m, 2H), 2.29-2.20 (m, 1H), 2.05-1.94 (m, 1H), 1.80 (dt, J=7.2, 4.8 Hz, 1H), 1.40 (s, 9H).

### Example 136B. (R)-5-(3-aminopyrrolidin-1-yl)-7-(4-(methylsulfonyl)phenyl)pyrrolo[2,1-f][1,2,4]triazin-4-amine, TFA

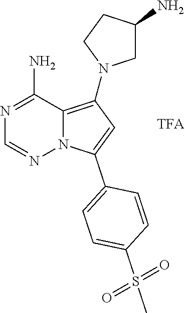 [SMILES: CS(=O)(=O)c1ccc(-c2cc(N3CC[C@@H](N)C3)c3c(N)ncnn23)cc1]

According to the procedure for the preparation of Example 112G, reaction of Example 136A afforded Example 136B (31 mg, 100% yield) as an off-white solid. MS: [M+H]⁺=372.95; ¹H NMR (500 MHz, DMSO-d₆) δ ppm 8.59 (br d, J=7.6 Hz, 1H), 8.36 (br d, J=8.5 Hz, 2H), 7.96 (br d, J=8.5 Hz, 2H), 7.88 (s, 1H), 7.61 (d, J=3.7 Hz, 1H), 7.21 (s, 1H), 6.85 (br d, J=3.4 Hz, 1H), 4.66-4.52 (m, 1H), 3.47-3.34 (m, 1H), 3.33-3.26 (m, 1H), 3.23 (s, 3H), 3.07-2.96 (m, 2H), 2.42-2.29 (m, 1H), 2.02-1.92 (m, 1H).

### Example 136

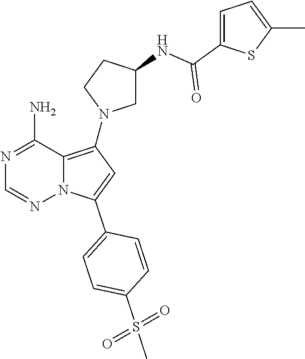 [SMILES: Cc1ccc(C(=O)N[C@@H]2CCN(c3cc(-c4ccc(S(C)(=O)=O)cc4)n4ncnc(N)c34)C2)s1]

According to the procedure for the preparation of Example 112, reaction of Example 136B and 5-methylthiophene-2-carboxylic acid afforded Example 136 (1.5 mg, 8% yield). LC-MS Method A: RT=1.387 min, [M+H]⁺=497.1; LC-MS Method B: RT=1.626 min, [M+H]⁺=497.1; ¹H NMR (500 MHz, DMSO-d₆) δ ppm 8.59 (br d, J=7.6 Hz, 1H), 8.36 (br d, J=8.5 Hz, 2H), 7.96 (br d, J=8.5 Hz, 2H), 7.88 (s, 1H), 7.61 (d, J=3.7 Hz, 1H), 7.21 (s, 1H), 6.85 (br d, J=3.4 Hz, 1H), 4.66-4.52 (m, 1H), 3.47-3.34 (m, 1H), 3.33-3.26 (m, 1H), 3.23 (s, 3H), 3.07-2.96 (m, 2H), 2.42-2.29 (m, 1H), 2.02-1.92 (m, 1H).

### Example 137

### Preparation of (R)—N-(1-(4-amino-7-(4-(methylsulfonyl)phenyl)pyrrolo[2,1-f][1,2,4]triazin-5-yl)pyrrolidin-3-yl)-5-chlorothiophene-2-carboxamide, TFA

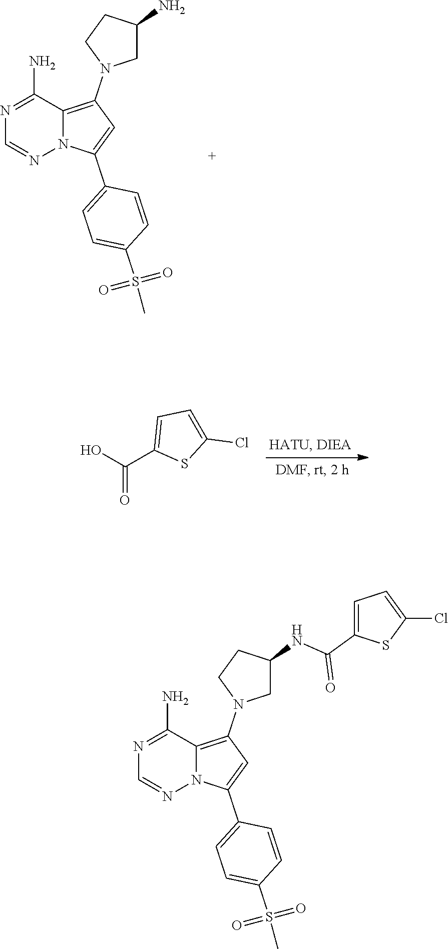 [SMILES: CS(=O)(=O)c1ccc(-c2cc(N3CC[C@@H](N)C3)c3c(N)ncnn23)cc1 ; O=C(O)c1ccc(Cl)s1 ; CS(=O)(=O)c1ccc(-c2cc(N3CC[C@@H](NC(=O)c4ccc(Cl)s4)C3)c3c(N)ncnn23)cc1]

According to the procedure for the preparation of Example 136, reaction of Example 136B and 5-chlorothiophene-2-carboxylic acid afforded Example 137 (2.3 mg, 8% yield). LC-MS Method A: RT=1.770 min, [M+H]⁺=517.1; LC-MS Method B: RT=1.130 min, [M+H]⁺=, 517.1 ¹H NMR (500 MHz, DMSO-d₆) δ ppm 8.41-8.24 (m, 2H), 8.07 (br dd, J=7.9, 4.3 Hz, 1H), 8.02-7.85 (m, 2H), 7.71-7.56 (m, 1H), 7.23-7.17 (m, 1H), 5.37-5.25 (m, 1H), 4.91-4.77 (m, 1H), 4.65-4.51 (m, 1H), 4.45-4.32 (m, 1H), 4.28-4.15 (m, 1H), 3.33-3.27 (m, 1H), 3.27-3.18 (m, 4H), 2.95-2.83 (m, 1H), 2.80-2.68 (m, 1H), 2.41-2.28 (m, 1H), 2.04-1.91 (m, 1H).

### Example 138

### Preparation of methyl (R)-3-(4-amino-5-(3-(5-chlorothiophene-2-carboxamido)piperidin-1-yl)pyrrolo[2,1-f][1,2,4]triazin-7-yl)-2,5-dihydro-1H-pyrrole-1-carboxylate, TFA

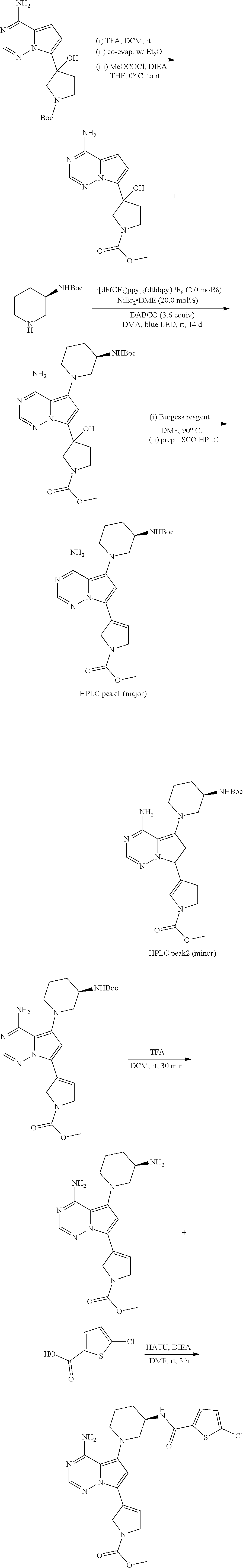

### Example 138A. Methyl 3-(4-amino-5-bromopyrrolo[2,1-f][1,2,4]triazin-7-yl)-3-hydroxypyrrolidine-1-carboxylate

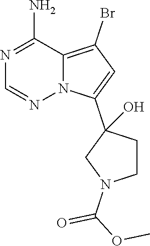 [SMILES: COC(=O)N1CCC(O)(c2cc(Br)c3c(N)ncnn23)C1]

Example 112B (100 mg, 0.251 mmol) was suspended in anhydrous DCM (2.5 mL), and was treated with TFA (2.5 mL). The reaction mixture was stirred at rt for 15 min. Solvent was removed under reduced pressure, the residue was co-evaporated with Et₂O (3×5 mL), and dried under vacuum to afford des-Boc intermediate as an off-white solid. The obtained material was dissolved/suspended in THF (5.0 mL) at 0° C., and DIEA (0.219 mL, 1.26 mmol) was added, followed by methyl chloroformate (0.021 mL, 0.28 mmol). The reaction mixture was stirred at 0° C. for 1 h, and then was allowed to reach at rt overnight. The reaction mixture was quenched with water (1.0 mL), and most of THF was removed under reduced pressure. The residue was diluted with EtOAc (150 mL) and brine (100 mL), organic phase was separated, washed with brine (1×50 mL), dried (Na₂SO₄), and filtered. EtOAc was removed under reduced pressure to afford Example 138A (86 mg, 96% yield) as an off-white solid. MS: [M+H]⁺=356.0; ¹H NMR (500 MHz, DMSO-d₆) δ ppm 7.89 (s, 1H), 6.78 (d, J=1.8 Hz, 1H), 5.66 (s, 1H), 3.80 (dd, J=11.2, 3.3 Hz, 1H), 3.71-3.65 (m, 1H), 3.60 (d, J=9.2 Hz, 3H), 3.55-3.42 (m, 2H), 2.64-2.52 (m, 1H), 2.16-2.07 (m, 1H).

### Example 138B. Methyl 3-(4-amino-5-((R)-3-((tert-butoxycarbonyl)amino)piperidin-1-yl)pyrrolo[2,1-f][1,2,4]triazin-7-yl)-3-hydroxypyrrolidine-1-carboxylate

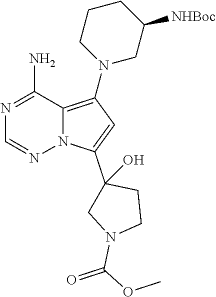 [SMILES: COC(=O)N1CCC(O)(c2cc(N3CCC[C@@H](NC(=O)OC(C)(C)C)C3)c3c(N)ncnn23)C1]

According to the procedure for the preparation of Example 112E, reaction of Example 138A and tert-butyl (R)-piperidin-3-ylcarbamate afforded Example 138B (35 mg, 30% yield) as a colorless film. MS: [M+H]⁺=476.2; ¹H NMR (500 MHz, DMSO-d₆) δ ppm 7.89-7.77 (m, 1H), 7.76-7.70 (m, 1H), 7.07-6.76 (m, 2H), 6.55 (d, J=2.7 Hz, 1H), 5.55 (s, 1H), 3.81 (dd, J=10.9, 2.1 Hz, 1H), 3.64 (br dd, J=11.2, 7.1 Hz, 1H), 3.59 (d, J=9.8 Hz, 3H), 3.55-3.40 (m, 2H), 3.07-2.99 (m, 1H), 2.91-2.79 (m, 1H), 2.62-2.54 (m, 1H), 2.14-2.05 (m, 1H), 1.85-1.56 (m, 3H), 1.38 (s, 9H), 1.24 (br s, 2H).

### Example 138C. Methyl (R)-3-(4-amino-5-(3-((tert-butoxycarbonyl)amino)piperidin-1-yl)pyrrolo[2,1-f][1,2,4]triazin-7-yl)-2,5-dihydro-1H-pyrrole-1-carboxylate

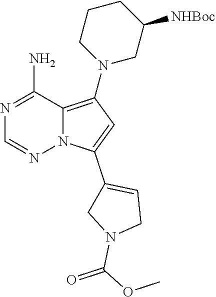 [SMILES: COC(=O)N1CC=C(c2cc(N3CCC[C@@H](NC(=O)OC(C)(C)C)C3)c3c(N)ncnn23)C1]

### Example 138D. Methyl (R)-4-(4-amino-5-(3-((tert-butoxycarbonyl)amino)piperidin-1-yl)pyrrolo[2,1-f][1,2,4]triazin-7-yl)-2,3-dihydro-1H-pyrrole-1-carboxylate

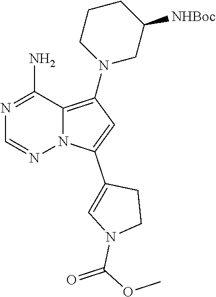 [SMILES: COC(=O)N1C=C(c2cc(N3CCC[C@@H](NC(=O)OC(C)(C)C)C3)c3c(N)ncnn23)CC1]

To a solution of Example 138B (35 mg, 0.074 mmol) in anhydrous DMF (2.0 mL) at rt, was added Burgess reagent (28.1 mg, 0.118 mmol). The reaction mixture was stirred at 90° C. for 1.0 h under nitrogen atmosphere. Additional amount of Burgess reagent (28.1 mg, 0.118 mmol) was added, and the reaction mixture was stirred at 90° C. for 16 h. The reaction mixture was cooled to rt, quenched with MeOH (2.0 mL). Then TFA (0.028 mL, 0.37 mmol) was added, and the residue was purified by preparative HPLC to afford the following compounds:

Example 138C (HPLC peak 1 (major); 20 mg, 0.044 mmol, 59.4% yield) as an off-white solid. MS: [M+H]⁺=458.25; ¹H NMR (500 MHz, DMSO-d₆) δ ppm 8.52-8.21 (m, 1H), 7.92 (s, 1H), 7.24-7.12 (m, 1H), 7.11-6.97 (m, 1H), 6.77 (br d, J=7.6 Hz, 1H), 6.76-6.72 (m, 1H), 4.54 (br dd, J=13.2, 3.0 Hz, 2H), 4.32-4.25 (m, 2H), 3.65 (d, J=5.6 Hz, 5H), 3.12-3.02 (m, 1H), 2.92-2.82 (m, 1H), 2.73-2.65 (m, 1H), 1.90-1.61 (m, 4H), 1.38 (s, 9H).

Example 138D (HPLC peak 2 (minor); (5.0 mg, 10.93 μmol, 14.9% yield) as an off-white solid. MS: [M+H]⁺=458.25; ¹H NMR (500 MHz, DMSO-d₆) δ ppm 7.96 (br s, 1H), 7.71-7.62 (m, 1H), 7.07-6.99 (m, 1H), 6.53 (s, 1H), 3.85-3.76 (m, 3H), 3.73-3.67 (m, 5H), 3.09-2.96 (m, 3H), 2.91-2.82 (m, 1H), 1.88-1.58 (m, 4H), 1.38 (s, 9H).

### Example 138E. Methyl (R)-3-(4-amino-5-(3-aminopiperidin-1-yl)pyrrolo[2,1-f][1,2,4]triazin-7-yl)-2,5-dihydro-1H-pyrrole-1-carboxylate, TFA

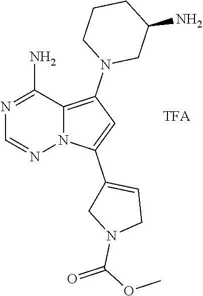 [SMILES: COC(=O)N1CC=C(c2cc(N3CCC[C@@H](N)C3)c3c(N)ncnn23)C1]

According to the procedure for the preparation of Example 112G, reaction of Example 138C afforded Example 138E (22 mg) as an off-white solid. MS: [M+H]⁺=358.10. Alternatively, Example 138E was prepared by the procedure described in Intermediate 32.

### Example 138

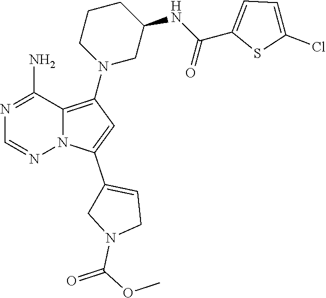 [SMILES: COC(=O)N1CC=C(c2cc(N3CCC[C@@H](NC(=O)c4ccc(Cl)s4)C3)c3c(N)ncnn23)C1]

According to the procedure for the preparation of Example 112, reaction of Example 138E and 5-chlorothiophene-2-carboxylic acid afforded Example 138 (2.9 mg, 20% yield). LC-MS Method A: RT=1.912 min, [M+H]⁺=502.3; LC-MS Method B: RT=1.568 min, [M+H]⁺=502.3 ¹H NMR (500 MHz, DMSO-d₆) δ ppm 8.48 (br d, J=7.8 Hz, 1H), 7.91 (s, 1H), 7.67 (d, J=4.1 Hz, 1H), 7.15 (d, J=4.1 Hz, 1H), 6.77 (d, J=5.1 Hz, 1H), 6.74 (br s, 1H), 4.56-4.48 (m, 2H), 4.32-4.23 (m, 2H), 4.14-4.05 (m, 1H), 3.67-3.60 (m, 3H), 3.20-3.12 (m, 1H), 3.04-2.97 (m, 1H), 2.73-2.60 (m, 2H), 1.94-1.87 (m, 1H), 1.87-1.73 (m, 2H), 1.56-1.43 (m, 1H).

### Example 139

### Preparation of (R)—N-(1-(4-amino-7-(4-carbamoyl-3-fluorophenyl)pyrrolo[2,1-f][1,2,4]triazin-5-yl)piperidin-3-yl)-5-chlorothiophene-2-carboxamide, TFA

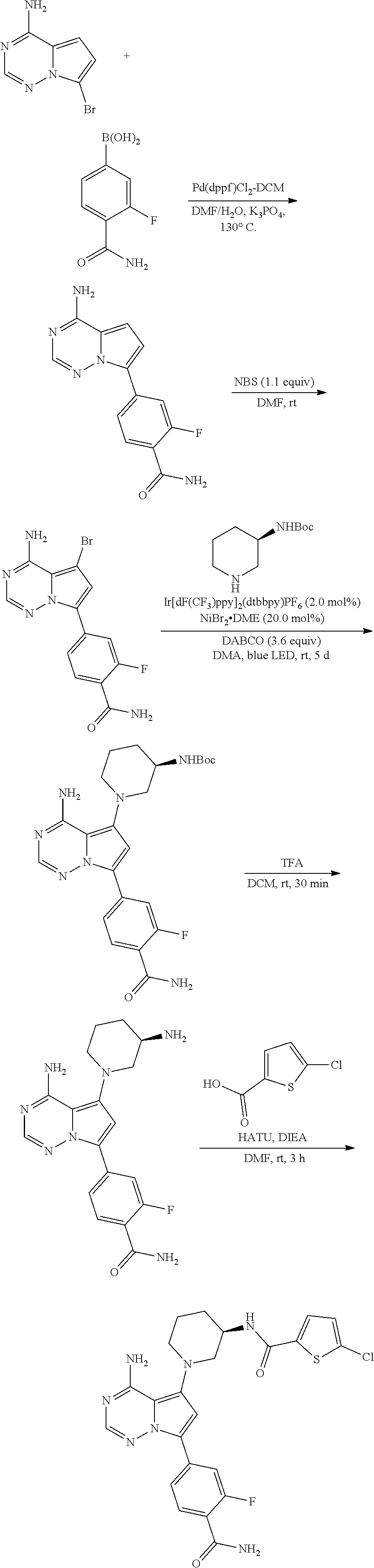

### Example 139A. 4-(4-Aminopyrrolo[2,1-f][1,2,4]triazin-7-yl)-2-fluorobenzamide

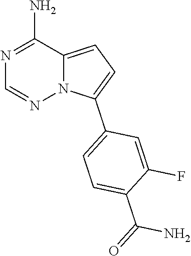 [SMILES: NC(=O)c1ccc(-c2ccc3c(N)ncnn23)cc1F]

Analogous to the procedure for the preparation of Example 1A, reaction of 7-bromopyrrolo[2,1-f][1,2,4]triazin-4-amine and (4-carbamoyl-3-fluorophenyl)boronic acid afforded Example 139A (159 mg, 62% yield) as a white solid. MS: [M+H]⁺=272.2; ¹H NMR (500 MHz, DMSO-d₆) δ ppm 8.14 (dd, J=13.0, 1.7 Hz, 1H), 8.05-7.98 (m, 2H), 7.95-7.79 (m, 2H), 7.75 (t, J=8.2 Hz, 1H), 7.68-7.58 (m, 2H), 7.25 (d, J=4.7 Hz, 1H), 7.04 (d, J=4.6 Hz, 1H); ¹⁹F NMR (471 MHz, DMSO-d₆) δ ppm −113.01 (s, 1F).

### Example 139B. 4-(4-Amino-5-bromopyrrolo[2,1-f][1,2,4]triazin-7-yl)-2-fluorobenzamide

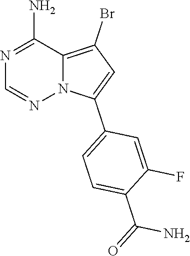 [SMILES: NC(=O)c1ccc(-c2cc(Br)c3c(N)ncnn23)cc1F]

Analogous to the procedure for the preparation of Example 1B, reaction of Example 139A afforded Example 139B (174 mg, 85% yield) as a grey solid. MS: [M+H]⁺=350.0; ¹H NMR (500 MHz, DMSO-d₆) δ ppm 8.08 (br d, J=12.8 Hz, 1H), 8.02 (s, 1H), 7.99 (br d, J=8.2 Hz, 1H), 7.74 (br t, J=8.1 Hz, 1H), 7.72-7.60 (m, 2H), 7.46 (s, 1H); ¹⁹F NMR (471 MHz, DMSO-d₆) δ ppm −112.98 (br s, 1F).

### Example 139C. tert-Butyl (R)-(1-(4-amino-7-(4-carbamoyl-3-fluorophenyl)pyrrolo[2,1-f][1,2,4]triazin-5-yl)piperidin-3-yl)carbamate

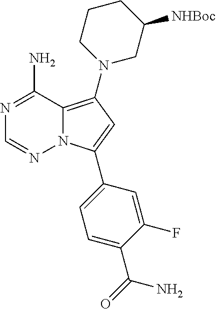 [SMILES: CC(C)(C)OC(=O)N[C@@H]1CCCN(c2cc(-c3ccc(C(N)=O)c(F)c3)n3ncnc(N)c23)C1]

According to the procedure for the preparation of Example 112E, reaction of Example 139B and tert-butyl (R)-piperidin-3-ylcarbamate afforded Example 139C (21 mg, 9% yield) as a yellowish solid. MS: [M+H]⁺=470.15; ¹H NMR (500 MHz, DMSO-d₆) δ ppm 8.36-8.19 (m, 1H), 8.11 (dd, J=13.0, 1.7 Hz, 1H), 8.01 (dd, J=8.2, 1.7 Hz, 1H), 7.96 (s, 1H), 7.74 (t, J=8.1 Hz, 1H), 7.65 (br d, J=17.9 Hz, 2H), 7.24 (s, 1H), 7.24-7.15 (m, 1H), 7.11-7.02 (m, 1H), 3.15-3.06 (m, 1H), 2.98-2.85 (m, 1H), 2.05-1.93 (m, 1H), 1.93-1.62 (m, 3H); ¹⁹F NMR (471 MHz, DMSO-d₆) δ ppm −113.00 (s, 1F).

### Example 139D. (R)-4-(4-amino-5-(3-aminopiperidin-1-yl)pyrrolo[2,1-f][1,2,4]triazin-7-yl)-2-fluorobenzamide, TFA

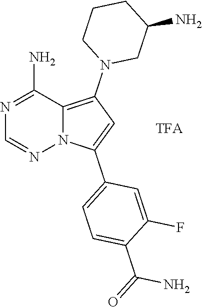 [SMILES: NC(=O)c1ccc(-c2cc(N3CCC[C@@H](N)C3)c3c(N)ncnn23)cc1F ; "TFA"]

According to the procedure for the preparation of Example 112G, reaction of Example 139C afforded Example 139D (22 mg) as an off-white solid. MS: [M+H]⁺=370.0.

### Example 139

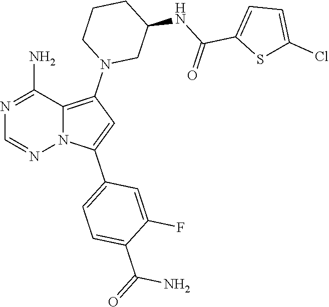 [SMILES: NC(=O)c1ccc(-c2cc(N3CCC[C@@H](NC(=O)c4ccc(Cl)s4)C3)c3c(N)ncnn23)cc1F]

According to the procedure for the preparation of Example 112, reaction of Example 139D and 5-chlorothiophene-2-carboxylic acid afforded Example 139 (10.3 mg, 33% yield). LC-MS Method A: RT=1.732 min, [M+H]⁺=514.2; LC-MS Method B: RT=1.435 min, [M+H]⁺=514.2; ¹H NMR (500 MHz, DMSO-d₆) δ ppm 8.50 (br d, J=7.6 Hz, 1H), 8.07 (br d, J=12.8 Hz, 1H), 8.00-7.91 (m, 2H), 7.78-7.72 (m, 1H), 7.72-7.67 (m, 2H), 7.63 (br s, 1H), 7.60 (br d, J=4.0 Hz, 1H), 7.25 (s, 1H), 7.21 (d, J=4.0 Hz, 1H), 7.16 (d, J=4.3 Hz, 1H), 4.22-4.07 (m, 1H), 3.27-3.19 (m, 1H), 3.12-3.01 (m, 1H), 2.79-2.66 (m, 2H), 1.98-1.76 (m, 3H), 1.60-1.44 (m, 1H); ¹⁹F NMR (471 MHz, DMSO-d₆) δ ppm −112.89 (br s, 1F).

### Example 140

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chloro-3-(2-methoxyethoxy)thiophene-2-carboxamide

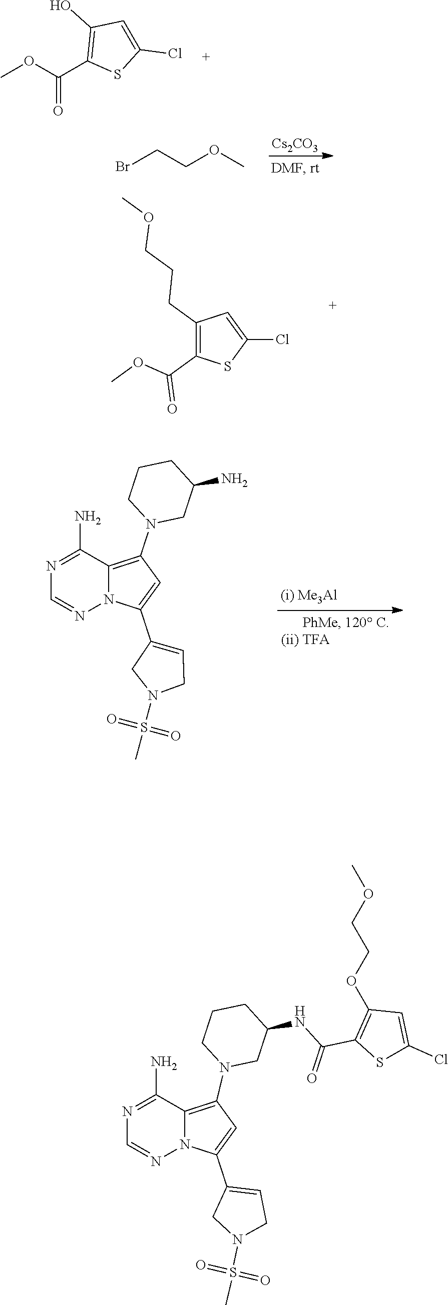

### Example 140A. Methyl 5-chloro-3-(2-methoxyethoxy)thiophene-2-carboxylate

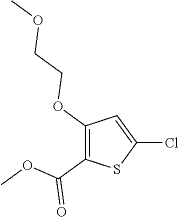

Methyl 5-chloro-3-hydroxythiophene-2-carboxylate (83 mg, 0.431 mmol) was dissolved in anhydrous DMF (4.0 mL), and cesium carbonate (281 mg, 0.862 mmol) was added. The reaction mixture was stirred at rt for 10 min. Afterwards, 1-bromo-2-methoxyethane (0.045 mL, 0.47 mmol) was added and the reaction mixture was stirred at rt for 16 h. The reaction mixture was quenched with TFA (0.332 mL, 4.31 mmol); caution: gas evolution), diluted with water to 6 mL, filtered and purified by preparative HPLC to give Example 140A (60 mg, 56% yield) as an off-white solid. MS: [M+H]⁺=250.9; ¹H NMR (500 MHz, DMSO-d₆) δ ppm. 7.33 (s, 1H), 4.29-4.26 (m, 2H), 3.72 (s, 3H), 3.65-3.60 (m, 2H), 3.31 (s, 3H).

### Example 140

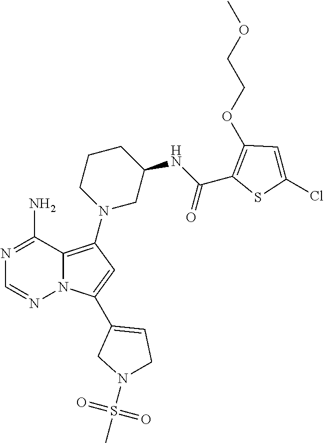 [SMILES: COCCOc1cc(Cl)sc1C(=O)N[C@@H]1CCCN(c2cc(C3=CCN(S(C)(=O)=O)C3)n3ncnc(N)c23)C1]

Example 112G (15 mg, 0.031 mmol) was suspended in anhydrous toluene (1 mL), then trimethylaluminum (2 M in toluene) (0.076 mL, 0.153 mmol) was added dropwise. After stirring for 5 min at rt, methyl 5-chloro-3-(2-methoxyethoxy)thiophene-2-carboxylate (8.4 mg, 0.034 mmol) was added, the vial was capped and the reaction mixture was stirred at 120° C. for 15 min under microwave irradiation. The reaction mixture was cooled to rt, diluted with MeOH (0.5 mL) and carefully quenched with TFA. Solvent was removed under reduced pressure, the residue was diluted with DMF (2 mL), filtered, and purified by preparative HPLC to afford Example 140 (1.6 mg, 8% yield). LC-MS Method A: RT=1.870 min, [M+H]⁺=596.1; LC-MS Method B: RT=1.588 min, [M+H]⁺=596.1; ¹H NMR (500 MHz, DMSO-d₆) δ ppm 8.11-7.87 (m, 1H), 7.84 (s, 1H), 7.68-7.45 (m, 1H), 7.31 (s, 1H), 6.74 (s, 1H), 6.70 (br s, 1H), 4.51 (br s, 2H), 4.39-4.30 (m, 2H), 4.28 (br s, 2H), 4.20-4.08 (m, 1H), 3.63 (br t, J=3.8 Hz, 1H), 3.52-3.40 (m, 2H), 2.98 (s, 1H), 2.93 (s, 4H), 1.90-1.72 (m, 3H).

### Example 141

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chloro-3-(2-(dimethylamino)ethoxy)thiophene-2-carboxamide, TFA

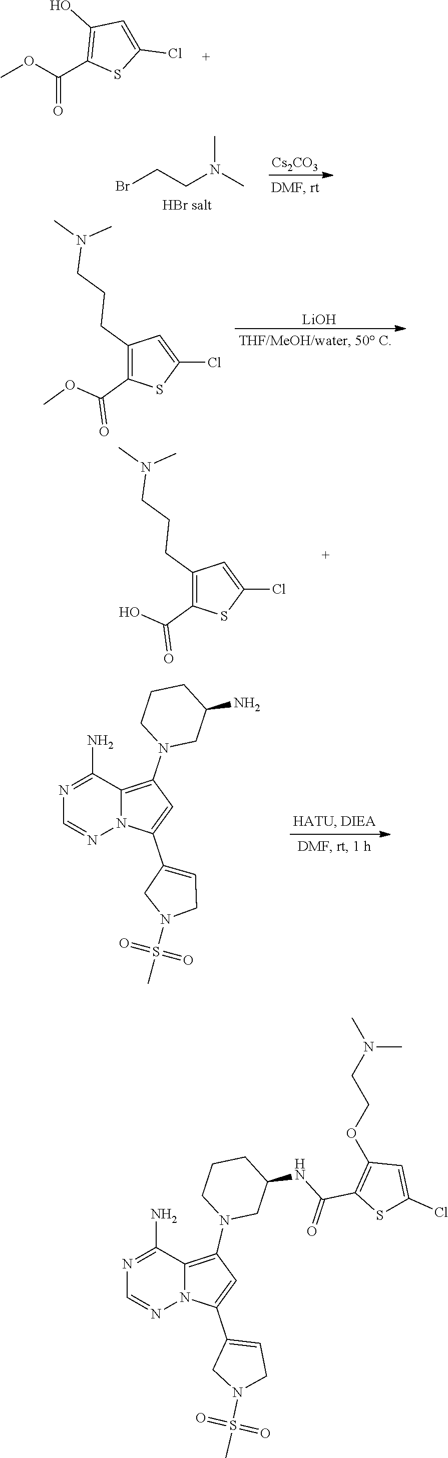

### Example 141A. Methyl 5-cgkiri-3-(2-(dimethylamino)ethoxy)thiophene-2-carboxylate, TFA.

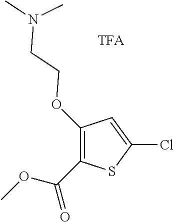 [SMILES: COC(=O)c1sc(Cl)cc1OCCN(C)C ; "TFA"]

Methyl 5-chloro-3-hydroxythiophene-2-carboxylate (83 mg, 0.431 mmol) was dissolved in DMF (4.0 mL), and cesium carbonate (421 mg, 1.293 mmol) was added. The reaction mixture was stirred at rt for 10 min. Afterwards, 2-bromo-N,N-dimethylethan-1-amine, hydrobromide (110 mg, 0.474 mmol) was added and the reaction mixture was stirred at rt for 16 h. The reaction mixture was quenched with TFA (0.332 mL, 4.31 mmol), diluted with water to 6 mL, filtered and purified by preparative HPLC to afford Example 141A (96 mg, 59% yield) as an off-white semisolid. MS: [M+H]⁺=263.9; ¹H NMR (500 MHz, DMSO-d₆) δ ppm 7.42 (s, 1H), 4.53-4.47 (m, 2H), 3.76-3.70 (m, 3H), 3.55-3.49 (m, 2H), 2.92 (s, 6H).

### Example 141B. 5-Chloro-3-(2-(dimethylamino)ethoxy)thiophene-2-carboxylic acid, TFA

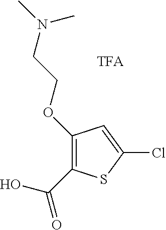

Example 141A (96 mg, 0.254 mmol) was dissolved in THF (1.6 mL) and MeOH (1.6 mL), then LiOH (1 M in water) (1.0 mL, 1.0 mmol) was added. The reaction was heated to 50° C. for 14 h. The reaction mixture was quenched with TFA (0.078 mL, 1.02 mmol), and concentrated under reduced pressure. The residue was diluted with DMF/water, and was purified by preparative HPLC to afford Example 141B (27 mg, 29% yield) as a white solid. MS: [M+H]⁺=249.85; ¹H NMR (500 MHz, DMSO-d₆) δ ppm 7.11 (s, 1H), 4.58-4.52 (m, 2H), 3.54-3.46 (m, 2H), 2.93 (s, 6H).

### Example 141

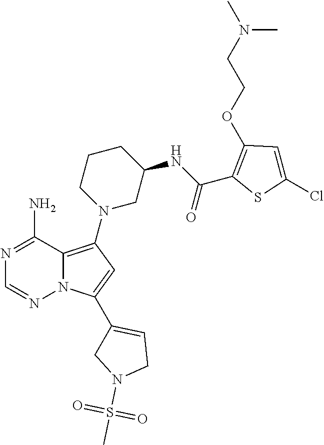 [SMILES: CN(C)CCOc1cc(Cl)sc1C(=O)N[C@@H]1CCCN(c2cc(C3=CCN(S(C)(=O)=O)C3)n3ncnc(N)c23)C1]

According to the procedure for the preparation of Example 112, reaction of Example 112G and Example 141B afforded Example 141 (14.1 mg, 60% yield). LC-MS Method A: RT=1.858 min, [M+H]⁺=609.0; LC-MS Method B: RT=1.152 min, [M+H]⁺=609.0; ¹H NMR (500 MHz, DMSO-d₆) δ ppm 7.90 (s, 1H), 7.34 (s, 1H), 7.25-7.19 (m, 1H), 6.71 (s, 2H), 4.60-4.50 (m, 4H), 4.50-4.45 (m, 1H), 4.29 (br s, 2H), 4.16-4.04 (m, 1H), 3.03-2.97 (m, 1H), 2.94 (s, 3H), 2.91 (s, 3H), 2.85 (s, 6H), 2.77-2.63 (m, 2H), 1.94-1.85 (m, 1H), 1.84-1.75 (m, 2H).

### Example 142

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chloro-3-(2-morpholinoethoxy)thiophene-2-carboxamide, TFA

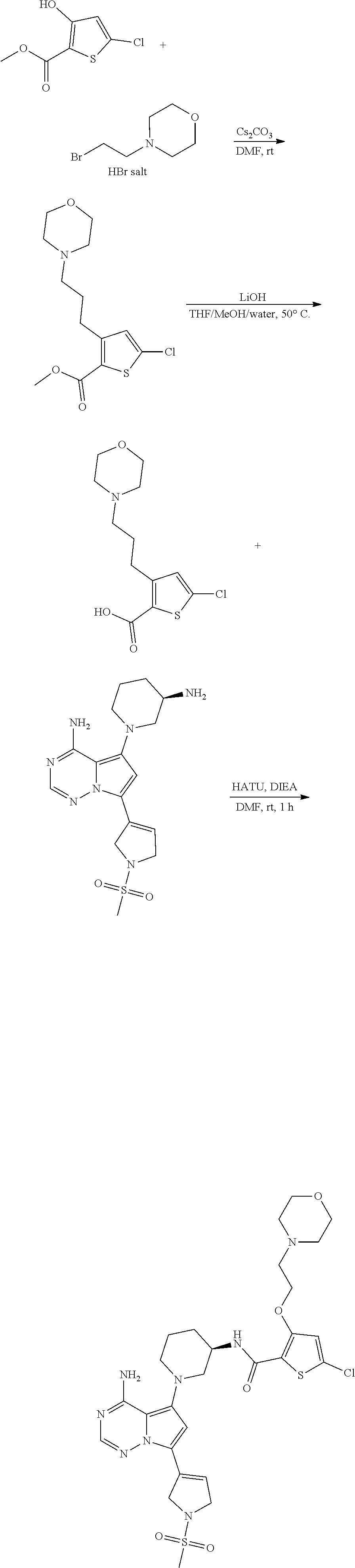

### Example 142A. Methyl 5-chloro-3-(2-morpholinoethoxy)thiophene-2-carboxylate, TFA

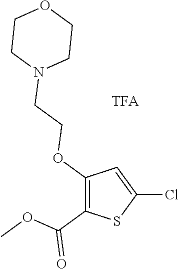 [SMILES: COC(=O)c1sc(Cl)cc1OCCN1CCOCC1 ; "TFA"]

According to the procedure for the preparation of Example 141A, reaction of methyl 5-chloro-3-hydroxythiophene-2-carboxylate and 4-(2-bromoethyl)morpholine, hydrobromide afforded Example 142A (174 mg, 96% yield) as an off-white semisolid. MS: [M+H]⁺=305.9, H NMR (500 MHz, DMSO-d₆) δ ppm 7.43 (s, 1H), 4.56-4.50 (m, 2H), 4.12-3.90 (m, 2H), 3.74 (s, 3H), 3.73-3.64 (m, 4H), 3.61-3.54 (m, 2H), 3.38-3.12 (m, 2H).

### Example 142B. 5-Chloro-3-(2-morpholinoethoxy)thiophene-2-carboxylic acid, TFA

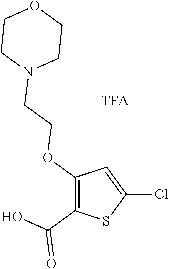 [SMILES: O=C(O)c1sc(Cl)cc1OCCN1CCOCC1 ; "TFA"]

According to the procedure for the preparation of Example 141B, reaction of Example 142A afforded Example 142B (109 mg, 65% yield) as a white solid. MS: [M+H]⁺=291.85; ¹H NMR (500 MHz, DMSO-d₆) δ ppm 9.37 (br s, 1H), 7.10 (s, 1H), 4.59-4.54 (m, 2H), 3.90 (t, J=4.8 Hz, 4H), 3.52-3.48 (m, 2H), 3.41 (br d, J=17.3 Hz, 4H).

### Example 142

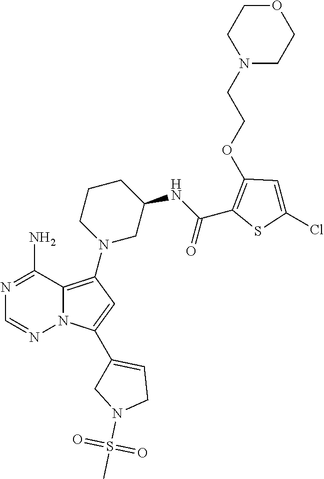 [SMILES: CS(=O)(=O)N1CC=C(c2cc(N3CCC[C@@H](NC(=O)c4sc(Cl)cc4OCCN4CCOCC4)C3)c3c(N)ncnn23)C1]

According to the procedure for the preparation of Example 141, reaction of Example 112G and Example 142B afforded Example 142 (17.5 mg, 66% yield). LC-MS Method A: RT=1.727 min, [M+H]⁺=651.1; LC-MS Method B: RT=1.170 min, [M+H]⁺=651.1; ¹H NMR (500 MHz, DMSO-d₆) δ ppm 7.93 (s, 1H), 7.32 (s, 1H), 7.29-7.21 (m, 1H), 6.74 (s, 2H), 4.55 (br s, 2H), 4.53-4.46 (m, 3H), 4.29 (br s, 2H), 4.18-4.05 (m, 1H), 3.63-3.51 (m, 1H), 3.39-3.22 (m, 1H), 2.93 (s, 3H), 2.78-2.63 (m, 2H), 1.94-1.86 (m, 1H), 1.84-1.74 (m, 2H), 1.59-1.44 (m, 1H).

### Example 143

### Preparation of rel-N-((3R,4S)-1-(4-amino-7-(4-(methylsulfonyl)phenyl)pyrrolo[2,1-f][1,2,4]triazin-5-yl)-4-fluoropiperidin-3-yl)-5-chlorothiophene-2-carboxamide, TFA

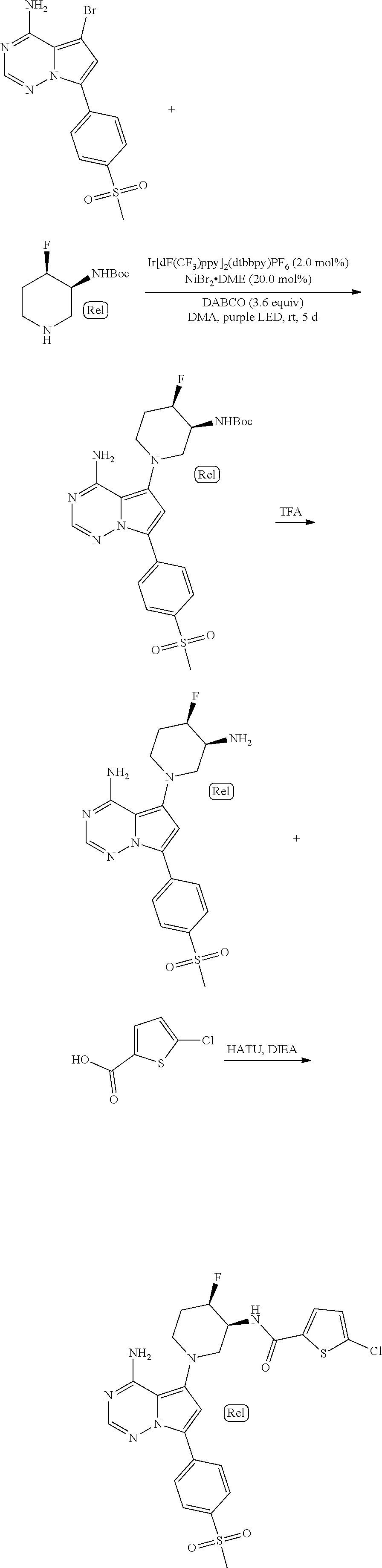

### Example 143A. rel-tert-Butyl ((3R,4S)-1-(4-amino-7-(4-(methylsulfonyl)phenyl)pyrrolo[2,1-f][1,2,4]triazin-5-yl)-4-fluoropiperidin-3-yl)carbamate

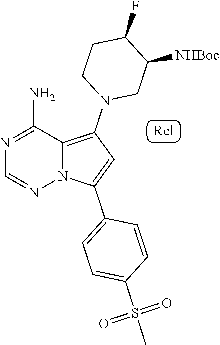 [SMILES: CC(C)(C)OC(=O)N[C@H]1CN(c2cc(-c3ccc(S(C)(=O)=O)cc3)n3ncnc(N)c23)CC[C@H]1F]

Analogous to the procedure for the preparation of Example 112E employing purple LED, reaction of Example 1B and rel-tert-butyl ((3R,4S)-4-fluoropiperidin-3-yl)carbamate afforded Example 143A (16 mg, 12% yield) as an off-white solid. MS: [M+H]⁺=505.35; ¹H NMR (500 MHz, DMSO-d₆) δ ppm 8.14-8.09 (m, 2H), 8.09-8.04 (m, 2H), 7.79 (s, 1H), 7.65 (br s, 1H), 6.94 (s, 1H), 5.03-4.86 (m, 2H), 3.31-3.20 (m, 1H), 3.05 (br d, J=7.5 Hz, 2H), 2.68 (s, 3H), 2.33-2.21 (m, 3H), 2.12-1.91 (m, 2H), 1.46 (s, 9H).

### Example 143B. rel-5-((3R,4S)-3-Amino-4-fluoropiperidin-1-yl)-7-(4-(methylsulfonyl)phenyl)pyrrolo[2,1-f][1,2,4]triazin-4-amine, TFA

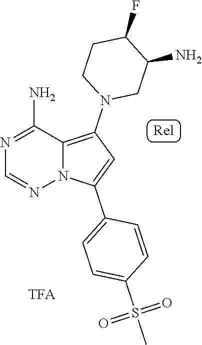 [SMILES: CS(=O)(=O)c1ccc(-c2cc(N3CC[C@@H](F)[C@@H](N)C3)c3c(N)ncnn23)cc1 ; "TFA"]

According to the procedure for the preparation of Example 112G, reaction of Example 143A afforded Example 143B (16 mg, 97% yield) as an off-white solid. MS: [M+H]⁺=405.0.

### Example 143

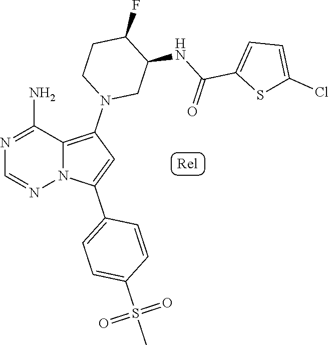 [SMILES: CS(=O)(=O)c1ccc(-c2cc(N3CC[C@@H](F)[C@@H](NC(=O)c4ccc(Cl)s4)C3)c3c(N)ncnn23)cc1]

According to the procedure for the preparation of Example 112, reaction of Example 143B and 5-chlorothiophene-2-carboxylic acid afforded Example 143 (10.6 mg, 49% yield) as an off-white solid. LC-MS Method A: RT=1.823 min, [M+H]⁺=549.0; LC-MS Method B: RT=1.546 min, [M+H]⁺=549.0; ¹H NMR (500 MHz, THF-d₈) δ ppm 8.63-8.46 (m, 1H), 8.33 (d, J=8.7 Hz, 2H), 7.99-7.93 (m, 2H), 7.89 (s, 1H), 7.70 (br d, J=8.4 Hz, 1H), 7.51 (d, J=4.0 Hz, 1H), 7.37-7.25 (m, 1H), 7.14 (s, 1H), 6.98 (d, J=4.0 Hz, 1H), 5.05-4.87 (m, 1H), 4.66-4.53 (m, 1H), 3.24 (dd, J=10.8, 4.9 Hz, 1H), 3.16-3.06 (m, 3H), 3.04 (s, 3H), 2.35-2.24 (m, 1H), 2.23-2.14 (m, 1H).

### Example 144

### Preparation of rel-N-((3R,4R)-1-(4-amino-7-(4-(methylsulfonyl)phenyl)pyrrolo[2,1-f][1,2,4]triazin-5-yl)-4-fluoropiperidin-3-yl)-5-chlorothiophene-2-carboxamide, TFA

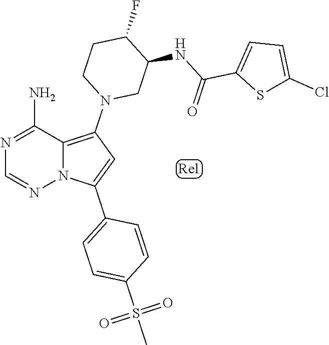 [SMILES: CS(=O)(=O)c1ccc(-c2cc(N3CC[C@H](F)[C@@H](NC(=O)c4ccc(Cl)s4)C3)c3c(N)ncnn23)cc1]

According to the procedure for the preparation of Example 143, using rel-tert-butyl ((3R,4R)-4-fluoropiperidin-3-yl)carbamate afforded Example 144 (10.7 mg, 36% yield) as a white solid. LC-MS Method A: RT=1.778 min, [M+H]⁺=548.95; LC-MS Method B: RT=1.517 min, [M+H]⁺=548.95; ¹H NMR (500 MHz, THF-d₈) δ ppm 8.36-8.30 (m, 2H), 7.97-7.92 (m, 2H), 7.87 (s, 1H), 7.73 (br d, J=7.2 Hz, 1H), 7.41 (d, J=4.1 Hz, 1H), 7.11 (s, 1H), 6.98 (d, J=4.0 Hz, 1H), 4.78-4.59 (m, 1H), 4.51-4.35 (m, 1H), 3.55-3.50 (m, 2H), 3.30 (br d, J=12.1 Hz, 2H), 3.03 (s, 3H), 3.00-2.91 (m, 1H), 2.90-2.82 (m, 1H), 2.33-2.23 (m, 1H), 2.21-2.11 (m, 1H).

### Example 145

### Preparation of N-(1-(4-amino-7-(4-(methylsulfonyl)phenyl)pyrrolo[2,1-f][1,2,4]triazin-5-yl)-3-methylpiperidin-3-yl)-5-chlorothiophene-2-carboxamide, TFA

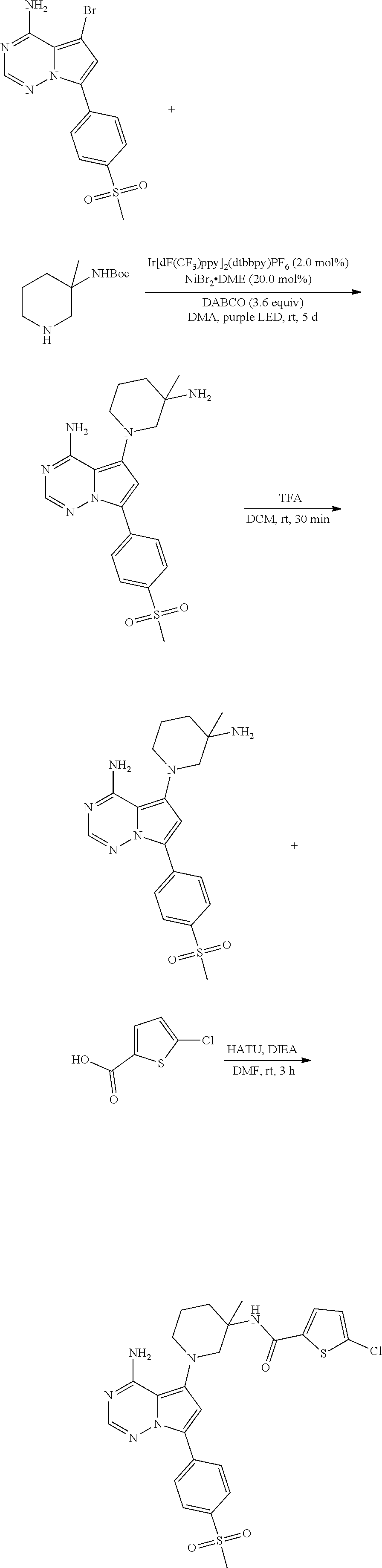 [SMILES: CS(=O)(=O)c1ccc(-c2cc(Br)c3c(N)ncnn23)cc1 ; CC1(NC(=O)OC(C)(C)C)CCCNC1 ; CC1(N)CCCN(c2cc(-c3ccc(S(C)(=O)=O)cc3)n3ncnc(N)c23)C1 ; CC1(N)CCCN(c2cc(-c3ccc(S(C)(=O)=O)cc3)n3ncnc(N)c23)C1 ; O=C(O)c1ccc(Cl)s1 ; CC1(NC(=O)c2ccc(Cl)s2)CCCN(c2cc(-c3ccc(S(C)(=O)=O)cc3)n3ncnc(N)c23)C1]

### Example 145A. ter-Butyl (1-(4-amino-7-(4-(methylsulfonyl)phenyl)pyrrolo[2,1-f][1,2,4]triazin-5-yl)-3-methylpiperidin-3-yl)carbamate

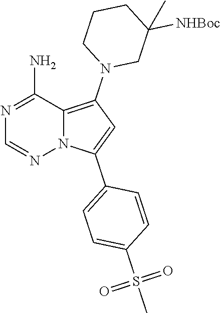 [SMILES: CC1(NC(=O)OC(C)(C)C)CCCN(c2cc(-c3ccc(S(C)(=O)=O)cc3)n3ncnc(N)c23)C1]

Analogous to the procedure for the preparation of Example 112E employing purple LED, reaction of Example 1B and tert-butyl (3-methylpiperidin-3-yl)carbamate afforded Example 145A (10 mg, 7% yield) as an off-white solid. MS: [M+H]⁺=501.25; ¹H NMR (500 MHz, DMSO-d₆) δ ppm 8.12-8.09 (m, 2H), 8.08 (d, J=3.5 Hz, 2H), 8.07-8.03 (m, 2H), 7.75 (s, 1H), 6.91 (s, 1H), 3.11 (s, 3H), 2.89-2.79 (m, 2H), 2.74 (br d, J=12.4 Hz, 1H), 1.44 (s, 9H), 1.43 (s, 4H).

### Example 145B. 5-(3-Amino-3-methylpiperidin-1-yl)-7-(4-(methylsulfonyl)phenyl)pyrrolo[2,1-f][1,2,4]triazin-4-amine, TFA

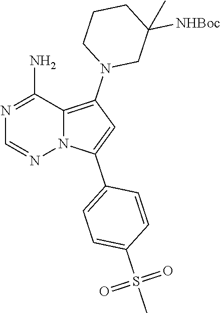 [SMILES: CC1(NC(=O)OC(C)(C)C)CCCN(c2cc(-c3ccc(S(C)(=O)=O)cc3)n3ncnc(N)c23)C1]

According to the procedure for the preparation of Example 112G, reaction of Example 145A afforded Example 145B (11 mg) as an off-white solid. MS: [M+H]⁺=401.0.

### Example 145

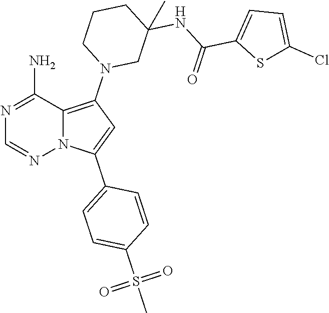 [SMILES: CC1(NC(=O)c2ccc(Cl)s2)CCCN(c2cc(-c3ccc(S(C)(=O)=O)cc3)n3ncnc(N)c23)C1]

According to the procedure for the preparation of Example 112, reaction of Example 145B and 5-chlorothiophene-2-carboxylic acid afforded Example 145 (3.4 mg, 23% yield) as an off-white solid. LC-MS Method A: RT=1.928 min, [M+H]⁺=545.0; LC-MS Method B: RT=1.518 min, [M+H]⁺=545.0; ¹H NMR (500 MHz, CD₃OD) δ ppm 8.19 (br d, J=8.3 Hz, 2H), 8.02 (d, J=8.5 Hz, 2H), 7.90 (s, 1H), 7.62 (br s, 1H), 7.60 (d, J=4.1 Hz, 1H), 7.48 (br d, J=3.6 Hz, 1H), 7.18 (s, 1H), 7.05 (d, J=4.1 Hz, 1H), 6.86 (br d, J=2.8 Hz, 1H), 4.07 (br d, J=11.8 Hz, 1H), 3.37-3.33 (m, 1H), 3.17 (s, 3H), 3.08 (br d, J=12.1 Hz, 2H), 2.27 (br d, J=12.7 Hz, 1H), 2.21-2.11 (m, 1H), 1.82-1.74 (m, 1H), 1.70-1.61 (m, 1H), 1.46 (s, 3H).

### Example 146

### Preparation of (R)—N-(1-(4-amino-7-(4-(methylsulfonyl)phenyl)pyrrolo[2,1-f][1,2,4]triazin-5-yl)-3-methylpiperidin-3-yl)-5-chlorothiophene-2-carboxamide, TFA

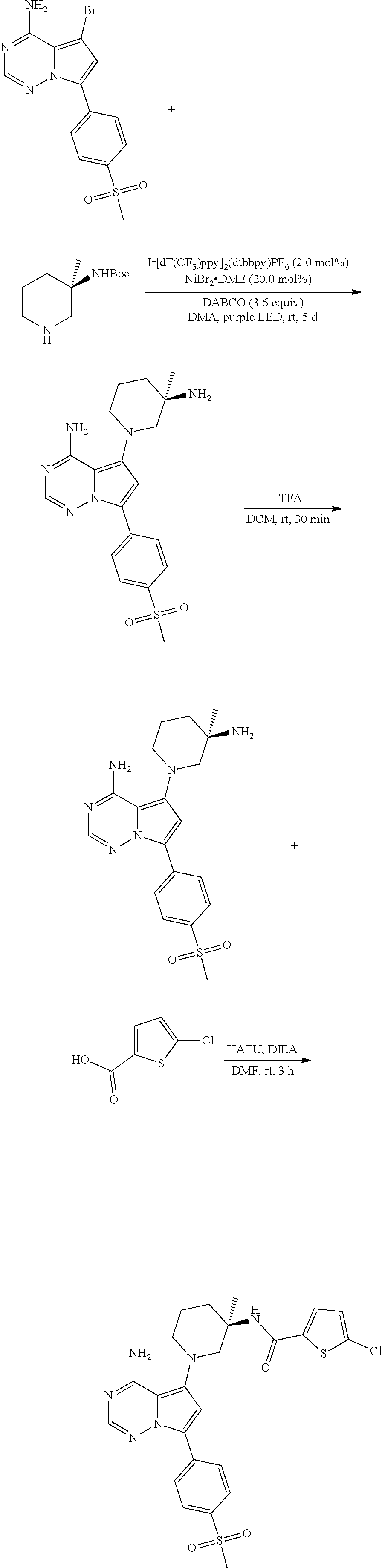 [SMILES: CS(=O)(=O)c1ccc(-c2cc(Br)c3c(N)ncnn23)cc1 ; CC(C)(C)OC(=O)N[C@]1(C)CCCNC1 ; C[C@@]1(N)CCCN(c2cc(-c3ccc(S(C)(=O)=O)cc3)n3ncnc(N)c23)C1 ; C[C@@]1(N)CCCN(c2cc(-c3ccc(S(C)(=O)=O)cc3)n3ncnc(N)c23)C1 ; O=C(O)c1ccc(Cl)s1 ; C[C@@]1(NC(=O)c2ccc(Cl)s2)CCCN(c2cc(-c3ccc(S(C)(=O)=O)cc3)n3ncnc(N)c23)C1]

### Example 146A. tert-Butyl (R)-(1-(4-amino-7-(4-(methylsulfonyl)phenyl)pyrrolo[2,1-f][1,2,4]triazin-5-yl)-3-methylpiperidin-3-yl)carbamate

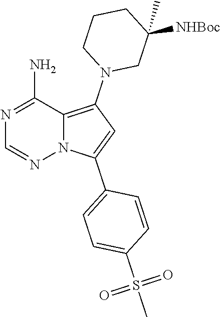 [SMILES: CC(C)(C)OC(=O)N[C@]1(C)CCCN(c2cc(-c3ccc(S(C)(=O)=O)cc3)n3ncnc(N)c23)C1]

Analogous to the procedure for the preparation of Example 112E employing purple LED, reaction of Example 1B and tert-butyl (R)-(3-methylpiperidin-3-yl)carbamate afforded Example 146A (16 mg, 12% yield) as an off-white solid. MS: [M+H]⁺=501.25; ¹H NMR (500 MHz, DMSO-d₆) δ ppm 8.12-8.09 (m, 2H), 8.08-8.02 (m, 3H), 7.75 (s, 1H), 6.91 (s, 1H), 3.11 (s, 4H), 3.11-3.06 (m, 1H), 2.86 (br d, J=7.9 Hz, 2H), 1.94-1.85 (m, 2H), 1.83-1.76 (m, 2H), 1.44 (s, 9H), 1.41 (br s, 3H).

### Example 146B. (R)-5-(3-Amino-3-methylpiperidin-1-yl)-7-(4-(methylsulfonyl)phenyl)pyrrolo[2,1-f][1,2,4]triazin-4-amine, TFA

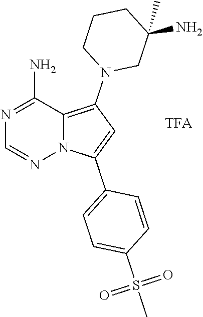 [SMILES: C[C@@]1(N)CCCN(c2cc(-c3ccc(S(C)(=O)=O)cc3)n3ncnc(N)c23)C1]

According to the procedure for the preparation of Example 112G, reaction of Example 146A afforded Example 146B (17 mg) as an off-white solid. MS: [M+H]⁺=401.2.

### Example 146

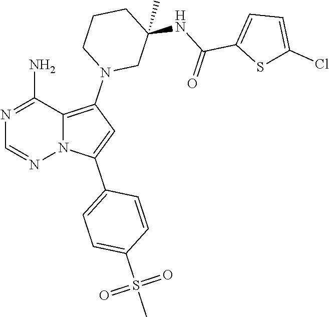 [SMILES: C[C@@]1(NC(=O)c2ccc(Cl)s2)CCCN(c2cc(-c3ccc(S(C)(=O)=O)cc3)n3ncnc(N)c23)C1]

According to the procedure for the preparation of Example 112, reaction of Example 146B and 5-chlorothiophene-2-carboxylic acid afforded Example 146 (3.4 mg, 23% yield) as an off-white solid. LC-MS Method A: RT=1.925 min, [M+H]⁺=545.0; LC-MS Method B: RT=1.518 min, [M+H]⁺=545.0; ¹H NMR (500 MHz, CD₃OD) δ ppm 8.22 (br d, J=8.5 Hz, 2H), 8.01 (d, J=8.5 Hz, 2H), 7.88 (s, 1H), 7.63 (br s, 1H), 7.49 (br d, J=3.6 Hz, 1H), 7.17 (s, 1H), 6.89 (br d, J=3.0 Hz, 1H), 4.00 (br d, J=12.1 Hz, 1H), 3.33 (br s, 1H), 3.17 (s, 3H), 3.11-2.99 (m, 2H), 2.29 (br d, J=12.4 Hz, 1H), 2.20-2.05 (m, 1H), 1.82-1.75 (m, 1H), 1.70-1.58 (m, 1H), 1.47 (s, 3H).

The following examples in Table 5 were prepared using the same procedure as shown in Example 130. Example 130D was coupled with the appropriate carboxylic acid. Various coupling reagents could be used other than the one described, such as BOP, PyBop, EDC/HOBt or T3P.

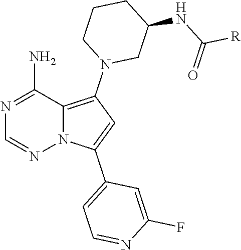

TABLE 5

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 147 | 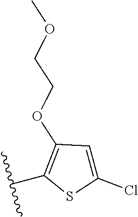 [SMILES: *c1sc(Cl)cc1OCCOC] | (R)-N-(1-(4-amino-7-(2- fluoropyridin-4- yl)pyrrolo[2,1- f][1,2,4]triazin-5- yl)piperidin-3-yl)-5-chloro- 3-(2- methoxyethoxy)thiophene- 2-carboxamide | 546.1 | A: 2.052 B: 1.697 | (500 MHz, DMSO-d₆) δ ppm 8.19 (br d, J = 5.2 Hz, 1H), 8.14- 8.02 (m, 1H), 8.00 (br d, J = 4.6 Hz, 1H), 7.90 (s, 2H), 7.68- 7.45 (m, 1H), 7.37 (s, 1H), 7.25 (s, 1H), 6.99-6.80 (m, 1H), 4.29 (br d, J = 4.3 Hz, 2H), 4.18-4.01 (m, 1H), 3.63-3.48 (m, 2H), 3.20 (br s, 1H), 1.91-1.67 (m, 3H), 1.60-1.35 (m, 1H) |
| 148 | 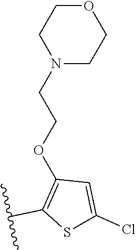 [SMILES: *c1sc(Cl)cc1OCCN1CCOCC1] | (R)-N-(1-(4-amino-7-(2- fluoropyridin-4- yl)pyrrolo[2,1- f][1,2,4]triazin-5- yl)piperidin-3-yl)-5-chloro- 3-(2- morpholinoethoxy) thiophene- 2-carboxamide, TFA | 601.3 | A: 1.711 B: 1.446 | (500 MHz, DMSO-d₆) δ ppm 8.26 (br d, J = 5.2 Hz, 1H), 8.05 (br d, J = 4.3 Hz, 1H), 7.98 (s, 1H), 7.95 (s, 1H), 7.38 (s, 2H), 7.32 (s, 2H), 4.51 (br s, 2H), 4.25-4.09 (m, 1H), 3.83-3.60 (m, 3H), 3.42-3.28 (m, 1H), 3.25-3.17 (m, 1H), 3.12-3.01 (m, 2H), 2.97-2.90 (m, 1H), 2.88-2.74 (m, 1H), 2.02-1.77 (m, 3H), 1.67-1.51 (m, 1H) |
| 149 | 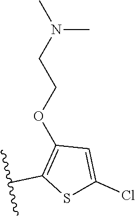 [SMILES: *c1sc(Cl)cc1OCCN(C)C] | (R)-N-(1-(4-amino-7-(2- fluoropyridin-4- yl)pyrrolo[2,1- f][1,2,4]triazin-5- yl)piperidin-3-yl)-5-chloro- 3-(2- (dimethylamino)ethoxy) thiophene- 2-carboxamide, TFA | 559.1 | A: 1.938 B: 1.299 | (500 MHz, DMSO-d₆) δ ppm 8.22 (d. J = 5.2 Hz, 1H), 8.03 (br d, J = 4.3 Hz, 1H), 7.96 (s, 1H), 7.92 (s, 1H), 7.36 (s, 1H), 7.32 (s, 1H), 7.22-7.18 (m, 1H), 4.52 (br d, J = 4.9 Hz, 2H), 4.16-4.05 (m, 1H), 3.06-2.96 (m, 1H), 2.81 (br s, 6H), 2.77-2.68 (m, 1H), 1.92-1.84 (m, 1H), 1.84-1.74 (m, 2H), 1.62-1.47 (m, 1H) |
| 150 | 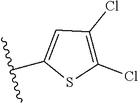 [SMILES: *c1cc(Cl)c(Cl)s1] | (R)-N-(1-(4-amino-7-(2- fluoropyridin-4- yl)pyrrolo[2,1- f][1,2,4]triazin-5- yl)piperidin-3-yl)-4,5- dichlorothiophene-2- carboxamide | 506.0 | A: 2.130 B: 1.795 | (500 MHz, DMSO-d₆) δ ppm 8.58 (br d, J = 7.0 Hz, 1H), 8.24 (d, J = 5.5 Hz, 1H), 8.20-8.10 (m, 1H), 8.08 (br d, J = 5.2 Hz, 1H), 7.96 (s, 2H), 7.91 (br s, 1H), 7.42 (s, 1H), 7.11-6.94 (m, 1H), 4.13 (br d, J = 5.2 Hz, 1H), 3.25-3.13 (m, 1H), 3.10- 2.97 (m, 1H), 2.85-2.62 (m, 1H), 1.90 (br d, J = 2.1 Hz, 2H), 1.87-1.75 (m, 1H), 1.61-1.42 (m, 1H) |
| 151 | 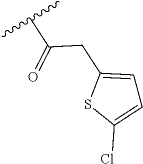 [SMILES: *C(=O)Cc1ccc(Cl)s1] | (R)-N-(1-(4-amino-7-(2- fluoropyridin-4- yl)pyrrolo[2,1- f][1,2,4]triazin-5- yl)piperidin-3-yl)-2-(5- chlorothiophen-2- yl)acetamide | 486.3 | A: 1.926 B: 1.651 | (500 MHz, DMSO-d₆) δ ppm 8.38-8.26 (m, 1H), 8.24 (d, J = 5.5 Hz, 1H), 8.18-8.09 (m, 1H), 8.07 (br d, J = 4.9 Hz, 1H), 7.96 (s, 2H), 7.40 (s, 1H), 7.10-6.96 (m, 1H), 6.90 (d, J = 3.7 Hz, 1H), 6.75 (d, J = 3.7 Hz, 1H), 4.02-3.92 (m, 1H), 3.65 (br d, J = 4.9 Hz, 1H), 3.40 (br d, J = 2.4 Hz, 2H), 3.03- 2.85 (m, 1H), 1.96-1.84 (m, 1H), 1.84-1.68 (m, 2H), 1.50- 1.30 (m, 1H) |
| 152 | 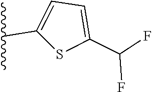 [SMILES: *c1ccc(C(F)F)s1] | (R)-N-(1-(4-amino-7-(2- fluoropyridin-4- yl)pyrrolo[2,1- f][1,2,4]triazin-5- yl)piperidin-3-yl)-5- (difluoromethyl)thiophene- 2-carboxamide, TFA | 488.3 | A: 1.940 B: 1.523 | |
| 153 | 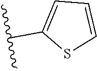 [SMILES: *c1cccs1] | (R)-N-(1-(4-amino-7-(2- fluoropyridin-4- yl)pyrrolo[2,1- f][1,2,4]triazin-5- yl)piperidin-3- yl)thiophene-2- carboxamide | 438.1 | A: 1.752 B: 1.442 | (500 MHz, DMSO-d₆) δ ppm 8.38 (br d, J = 7.9 Hz, 1H), 8.24 (d, J = 5.4 Hz, 1H), 8.20-8.11 (m, 1H), 8.08 (br d, J = 5.4 Hz, 1H), 7.97 (s, 2H), 7.81 (d, J = 3.2 Hz, 1H), 7.76-7.70 (m, 1H), 7.41 (s, 1H), 7.14 (dd, J = 4.9, 3.9 Hz, 1H), 7.11-6.94 (m, 1H), 4.24-4.09 (m, 1H), 3.29-3.20 (m, 1H), 3.10- 3.00 (m, 1H), 2.78-2.64 (m, 1H), 1.98-1.74 (m, 3H), 1.62- 1.45 (m, 1H) |
| 154 | 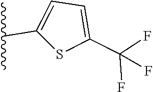 [SMILES: *c1ccc(C(F)(F)F)s1] | (R)-N-(1-(4-amino-7-(2- fluoropyridin-4- yl)pyrrolo[2,1- f][1,2,4]triazin-5- yl)piperidin-3-yl)-5- (trifluoromethyl)thiophene- 2-carboxamide, TFA | 506.1 | A: 2.074 B: 1.746 | (500 MHz, DMSO-d₆) δ ppm 8.74 (br d, J = 7.6 Hz, 1H), 8.26 (d, J = 5.6 Hz, 1H), 8.08 (br d, J = 3.4 Hz, 1H), 8.00 (d, J = 1.1 Hz, 1H), 7.96 (s, 1H), 7.90 (br s, 1H), 7.75 (br s, 1H), 7.44 (d. J = 2.3 Hz, 1H), 4.25-4.13 (m, 1H), 3.23 (br dd, J = 10.6, 3.6 Hz, 1H), 3.11-3.02 (m, 1H), 2.80-2.66 (m, 1H), 2.00-1.77 (m, 3H), 1.62-1.48 (m, 1H) |
| 155 | 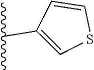 [SMILES: *c1ccsc1] | (R)-N-(1-(4-amino-7-(2- fluoropyridin-4- yl)pyrrolo[2,1- f][1,2,4]triazin-5- yl)piperidin-3- yl)thiophone-3- carboxamide | 438.3 | A: 1.742 B: 1.437 | (500 MHz, DMSO-d₆) δ ppm 8.24 (d, J = 5.5 Hz, 1H), 8.19 (br d, J = 7.9 Hz, 1H), 8.14 (br d, J = 1.5 Hz, 1H), 8.08 (br d, J = 5.2 Hz, 1H), 7.97 (s, 2H), 7.56 (dd, J = 4.7, 2.9 Hz, 1H), 7.50 (d, J = 4.9 Hz, 1H), 7.41 (s, 1H), 7.12-6.95 (m, 1H), 4.22-4.09 (m, 1H), 3.39 (br dd, J = 7.2, 2.3 Hz, 1H), 3.27- 3.18 (m, 1H), 3.10-3.01 (m, 1H), 2.77-2.63 (m, 1H), 2.01- 1.74 (m, 3H), 1.60-1.47 (m, 1H) |
| 156 | 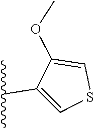 [SMILES: *c1cscc1OC] | (R)-N-(1-(4-amino-7-(2- fluoropyridin-4- yl)pyrrolo[2,1- f][1,2,4]triazin-5- yl)piperidin-3-yl)-4- methoxythiophene-3- carboxamide | 468.3 | A: 1.862 B: 1.548 | (500 MHz, DMSO-d₆) δ ppm 8.25 (d, J = 5.4 Hz, 1H), 8.23- 8.16 (m, 1H), 8.08 (br d, J = 5.3 Hz, 1H), 8.02 (d, J = 3.4 Hz, 1H), 7.97 (d, J = 5.5 Hz, 2H), 7.89-7.61 (m, 1H), 7.44 (s, 1H), 7.03-6.86 (m, 1H), 6.79 (d. J = 3.5 Hz, 1H), 4.30-4.15 (m, 1H), 3.89 (s, 3H), 3.45-3.35 (m, 3H), 3.08-2.63 (m, 1H), 1.89-1.71 (m, 3H), 1.66-1.46 (m, 1H) |
| 157 | 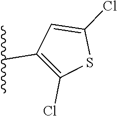 [SMILES: *c1cc(Cl)sc1Cl] | (R)-N-(1-(4-amino-7-(2- fluoropyridin-4- yl)pyrrolo[2,1- f][1,2,4]triazin-5- yl)piperidin-3-yl)-2,5- dichlorothiophene-3- carboxamide | 506.0 | A: 2.202 B: 1.906 | (500 MHz, DMSO-d₆) δ ppm 8.39 (br dd, J = 5.0, 2.0 Hz, 1H), 8.25 (d, J = 5.2 Hz, 1H), 8.21-8.14 (m, 1H), 8.09 (br d, J = 4.6 Hz, 1H), 7.98 (s, 2H), 7.43 (s, 1H), 7.33 (s, 1H), 7.11- 6.97 (m, 1H), 4.19-4.07 (m, 1H), 3.05-2.87 (m, 1H), 2.82-2.66 (m, 1H), 1.96-1.83 (m, 2H), 1.83-1.69 (m, 1H), 1.60-1.40 (m, 1H) |
| 158 | 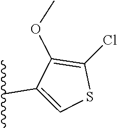 [SMILES: *c1csc(Cl)c1OC] | (R)-N-(1-(4-amino-7-(2- fluoropyridin-4- yl)pyrrolo[2,1- f][1,2,4]triazin-5- yl)piperidin-3-yl)-5-chloro- 4-methoxythiophene-3- carboxamide | 502.3 | A: 2.065 B: 1.729 | (500 MHz, DMSO-d₆) δ ppm 8.25 (d, J = 5.4 Hz, 1H), 8.23- 8.15 (m, 1H), 8.08 (br d, J = 4.8 Hz, 1H), 8.06 (br d, J = 2.7 Hz, 1H), 8.02-7.99 (m, 1H), 7.97 (d, J = 3.2 Hz, 1H), 7.84 (s, 1H), 7.43 (s, 1H), 7.12-6.91 (m, 1H), 4.23-4.13 (m, 1H), 3.87 (s, 3H), 3.48-3.40 (m, 2H), 3.02-2.74 (m, 1H), 1.95-1.84 (m, 2H), 1.84-1.72 (m, 1H), 1.63-1.47 (m, 1H) |
| 159 | 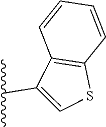 [SMILES: *c1csc2ccccc12] | (R)-N-(1-(4-amino-7-(2- fluoropyridin-4- yl)pyrrolo[2,1- f][1,2,4]triazin-5- yl)piperidin-3- yl)benzo[b]thiophene-3- carboxamide, TFA | 488.1 | A: 2.094 B: 1.826 | (500 MHz, DMSO-d₆) δ ppm 8.45 (br d, J = 6.7 Hz, 1H), 8.37 (br d, J = 7.9 Hz, 1H), 8.33 (s, 1H), 8.25 (d, J = 5.5 Hz, 1H), 8.09 (br d, J = 5.2 Hz, 1H), 8.02 (br d, J = 7.6 Hz, 1H), 8.01- 7.99 (m, 1H), 7.97 (s, 1H), 7.45 (s, 1H), 7.44-7.36 (m, 2H), 4.32-4.19 (m, 1H), 3.10-3.01 (m, 1H), 2.85-2.69 (m, 1H), 2.03-1.89 (m, 2H), 1.89-1.76 (m, 1H), 1.67-1.52 (m, 1H) |
| 160 | 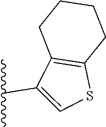 [SMILES: *c1csc2c1CCCC2] | (R)-N-(1-(4-amino-7-(2- fluoropyridin-4- yl)pyrrolo[2,1- f][1,2,4]triazin-5- yl)piperidin-3-yl)-4,5,6,7- tetrahydrobenzo[b] thiophene-3- carboxamide, TFA | 492.2 | A: 2.203 B: 1.926 | (500 MHz, DMSO-d₆) δ ppm 8.25 (d, J = 5.5 Hz, 1H), 8.12- 8.03 (m, 2H), 7.98 (d, J = 7.6 Hz, 2H), 7.70 (s, 1H), 7.43 (s, 1H), 4.18-4.03 (m, 1H), 3.26-3.14 (m, 1H), 2.76 (br d, J = 4.6 Hz, 1H), 2.69 (br d, J = 6.1 Hz, 4H), 1.88 (br d, J = 5.2 Hz, 2H), 1.82-1.70 (m, 3H), 1.70-1.62 (m, 2H), 1.56- 1.40 (m, 1H) |
| 161 | 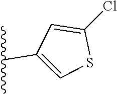 [SMILES: *c1csc(Cl)c1] | (R)-N-(1-(4-amino-7-(2- fluoropyridin-4- yl)pyrrolo[2,1- f][1,2,4]triazin-5- yl)piperidin-3-yl)-5- chlorothiophone-3- carboxamide | 472.0 | A: 1.943 B: 1.567 | (500 MHz, DMSO-d₆) δ ppm 8.28-8.20 (m, 2H), 8.17- 8.09 (m, 1H), 8.07 (br d, J = 4.9 Hz, 1H), 8.01 (s, 1H), 7.96 (s, 2H), 7.50 (s, 1H), 7.40 (s, 1H), 7.13-6.92 (m, 1H), 4.18- 4.06 (m, 1H), 3.24-3.14 (m, 1H), 3.07-2.97 (m, 1H), 2.80-2.62 (m, 1H), 1.96-1.84 (m, 2H), 1.85-1.71 (m, 1H), 1.60-1.44 (m, 1H) |

The following Examples in Table 6 were prepared using the same procedure as shown in Example 139. Example 139D was coupled with the appropriate carboxylic acid. Various coupling reagents could be used other than the one described, such as BOP, PyBop, EDC/HOBt or T3P.

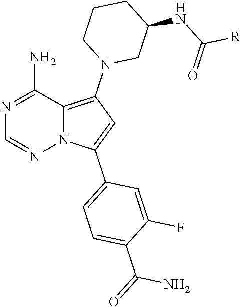

TABLE 6

| | | | LCMS | HPLC | 1HNMR |
|---|---|---|---|---|---|
| | | | (M + H)⁺ | Method, | |
| | Structure | | | RT | |
| Example | (R) | Name | | (min.) | |
| 162 | 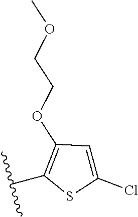 [SMILES: *c1sc(Cl)cc1OCCOC] | (R)-N-(1-(4-amino-7- (4-carbamoyl-3- fluorophenyl)pyrrolo [2,1-f][1,2,4]triazin- 5-yl)piperidin-3-yl)- 5-chloro-3-(2- methoxyethoxy) thiophene- 2-carboxamide | 588.0 | A: 1.683 B: 1.638 | (500 MHz, DMSO-d₆) δ ppm 8.11 (br d, J = 12.5 Hz, 1H), 8.00 (br d, J = 8.5 Hz, 1H), 7.91 (s, 1H), 7.73 (br t. J = 8.1 Hz, 1H), 7.66 (br s, 1H), 7.62 (br s, 1H), 7.33 (s, 1H), 7.26 (s, 1H), 6.98-6.81 (m, 1H), 4.42-4.30 (m, 2H), 4.25-4.12 (m, 1H), 3.65 (br d, J = 3.4 Hz, 2H), 3.40-3.32 (m, 1H), 3.04-2.93 (m, 1H), 1.96-1.75 (m, 3H), 1.62- 1.43 (m, 1H) |
| 163 | 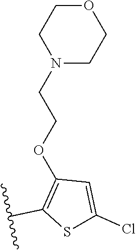 [SMILES: *c1sc(Cl)cc1OCCN1CCOCC1] | (R)-N-(1-(4-amino-7- (4-carbamoyl-3- fluorophenyl)pyrrolo [2,1-f][1,2,4]triazin- 5-yl)piperidin-3-yl)- 5-chloro-3-(2- morpholinoethoxy) thiophene-2- carboxamide, TFA | 643.0 | A: 1.801 B: 1.227 | (500 MHz, DMSO-d₆) δ ppm 8.08 (br d, J = 12.2 Hz, 1H), 8.01-7.95 (m, 2H), 7.75 (br t, J = 7.9 Hz, 1H), 7.68 (br s, 1H), 7.65 (br s, 1H), 7.37 (s, 1H), 7.30-7.25 (m, 1H), 7.22 (s, 1H), 4.59 (br s, 1H), 4.55-4.49 (m, 1H), 4.23- 4.10 (m, 1H), 3.29-3.20 (m, 1H), 3.09-2.99 (m, 1H), 2.84-2.72 (m, 1H), 2.54 (s, 4H), 1.97-1.88 (m, 1H), 1.88-1.77 (m, 2H), 1.66-1.48 (m, 1H) |
| 164 | 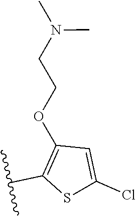 [SMILES: *c1sc(Cl)cc1OCCN(C)C] | (R)-N-(1-(4-amino-7- (4-carbamoyl-3- fluorophenyl)pyrrolo [2,1-f][1,2,4]triazin- 5-yl)piperidin-3-yl)- 5-chloro-3-(2- (dimethylamino) ethoxy)thiophene-2- carboxamide, 2 TFA | 601.1 | A: 1.750 B: 1.173 | (500 MHz, DMSO-d₆) δ ppm 8.43 (br d, J = 13.1 Hz, 1H), 8.36-8.24 (m, 2H), 8.08 (br t, J = 7.8 Hz, 1H), 7.99 (br d, J = 16.5 Hz, 2H), 7.70 (s, 1H), 7.58-7.52 (m, 1H), 4.89 (br s, 2H), 4.54-4.41 (m, 1H), 3.63-3.52 (m, 1H), 3.44- 3.33 (m, 1H), 3.19 (br s, 6H), 3.13-3.01 (m, 1H), 2.87 (s, 1H), 2.24 (br dd, J = 9.8, 7.6 Hz, 1H), 2.22-2.07 (m, 2H), 1.99-1.81 (m, 1H) |

### Example 165

### Preparation of rel-N-((1R,6R)-3-(4-amino-7-(4-(methylsulfonyl)phenyl)pyrrolo[2,1-f][1,2,4]triazin-5-yl)-3-azabicyclo[4.1.0]heptan-1-yl)-5-chlorothiophene-2-carboxamide, TFA

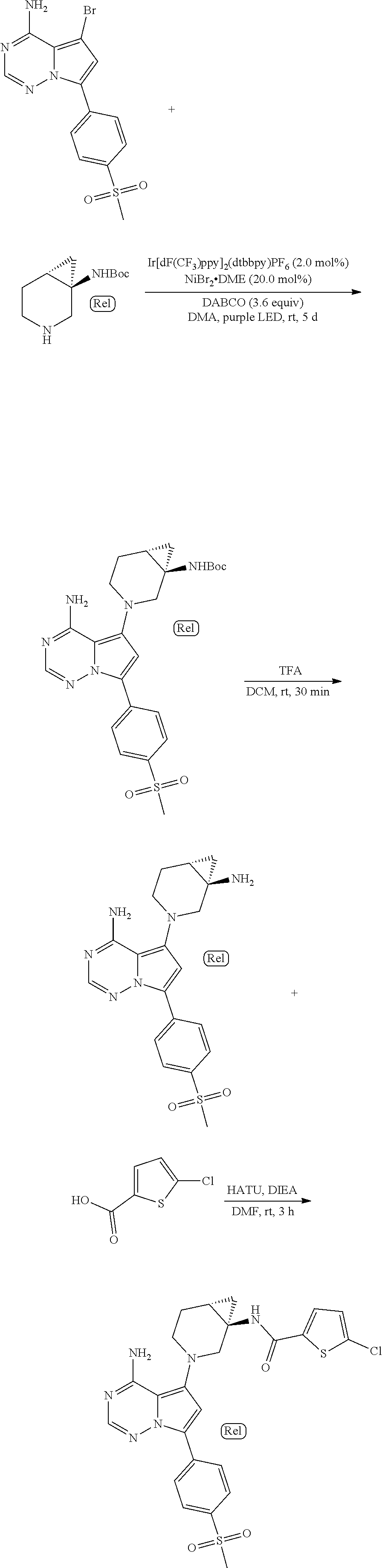 [SMILES: CS(=O)(=O)c1ccc(-c2cc(Br)c3c(N)ncnn23)cc1 ; CC(C)(C)OC(=O)N[C@@]12CNCC[C@@H]1C2 ; CC(C)(C)OC(=O)N[C@]12C[C@H]1CCN(c1cc(-c3ccc(S(C)(=O)=O)cc3)n3ncnc(N)c13)C2 ; CS(=O)(=O)c1ccc(-c2cc(N3CC[C@@H]4C[C@]4(N)C3)c3c(N)ncnn23)cc1 ; O=C(O)c1ccc(Cl)s1 ; CS(=O)(=O)c1ccc(-c2cc(N3CC[C@@H]4C[C@]4(NC(=O)c4ccc(Cl)s4)C3)c3c(N)ncnn23)cc1]

### Example 165A. rel-tert-Butyl ((1R,6R)-3-(4-amino-7-(4-(methylsulfonyl)phenyl)pyrrolo[2,1-f][1,2,4]triazin-5-yl)-3-azabicyclo[4.1.0]heptan-1-yl)carbamate

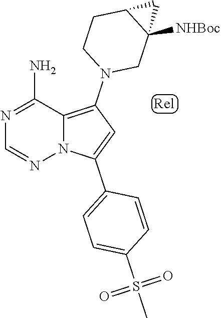 [SMILES: CC(C)(C)OC(=O)N[C@]12C[C@H]1CCN(c1cc(-c3ccc(S(C)(=O)=O)cc3)n3ncnc(N)c13)C2]

Analogous to the procedure for the preparation of Example 112E employing purple LED, reaction of Example 1B and rel-tert-butyl ((1R,6R)-3-azabicyclo[4.1.0]heptan-1-yl)carbamate afforded Example 165A (10 mg, 7% yield) as a brown solid. MS: [M+H]⁺=499.20; ¹H NMR (500 MHz, DMSO-d₆) δ ppm 8.38-8.34 (m, 1H), 8.32 (br d, J=8.4 Hz, 1H), 8.09-8.03 (m, 2H), 7.97 (d, J=5.6 Hz, 1H), 3.61 (d, J=11.6 Hz, 1H), 3.49-3.42 (m, 1H), 3.41-3.35 (m, 2H), 3.23-3.20 (m, 1H), 3.20 (s, 3H), 3.05-2.95 (m, 1H), 2.94-2.86 (m, 1H), 2.85-2.78 (m, 1H), 2.45-2.33 (m, 1H), 2.04-1.92 (m, 1H), 1.55-1.42 (m, 9H), 1.34-1.28 (m, 1H), 1.17 (t, J=6.6 Hz, 1H).

### Example 165B. rel-5-((1R,6R)-1-Amino-3-azabicyclo[4.1.0]heptan-3-yl)-7-(4-(methylsulfonyl)phenyl)pyrrolo[2,1-f][1,2,4]triazin-4-amine, 2 TFA

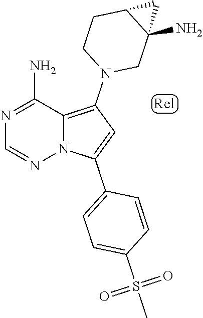 [SMILES: CS(=O)(=O)c1ccc(-c2cc(N3CC[C@@H]4C[C@]4(N)C3)c3c(N)ncnn23)cc1]

According to the procedure for the preparation of Example 112G, reaction of Example 165A afforded Example 165B (13 mg) as an off-white solid. MS: [M+H]⁺=399.1.

### Example 165

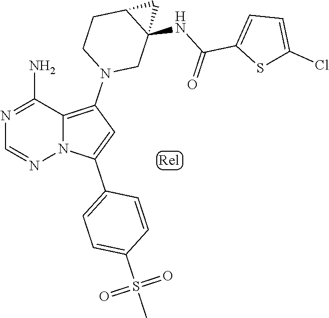 [SMILES: CS(=O)(=O)c1ccc(-c2cc(N3CC[C@@H]4C[C@]4(NC(=O)c4ccc(Cl)s4)C3)c3c(N)ncnn23)cc1]

According to the procedure for the preparation of Example 112, reaction of Example 165B and 5-chlorothiophene-2-carboxylic acid afforded Example 165 (3.4 mg, 21% yield). LC-MS Method A: RT=1.976 min, [M+H]⁺=543.0; LC-MS Method B: RT=1.662 min, [M+H]⁺=543.0; ¹H NMR (500 MHz, DMSO-d₆) δ ppm 9.10 (s, 1H), 8.35 (br d, J=8.6 Hz, 2H), 7.98-7.93 (m, 3H), 7.62 (d, J=4.1 Hz, 1H), 7.25 (d, J=8.1 Hz, 2H), 7.16 (d, J=4.0 Hz, 1H), 7.14 (s, 1H), 7.04 (s, 1H), 3.43-3.35 (m, 1H), 3.31-3.24 (m, 1H), 3.23 (s, 2H), 2.86-2.77 (m, 2H), 2.56 (br d, J=11.4 Hz, 2H), 2.28-2.18 (m, 1H), 1.93-1.81 (m, 1H), 1.35-1.28 (m, 1H), 1.28-1.19 (m, 2H), 1.03 (br dd, J=9.7, 5.1 Hz, 1H), 0.91 (br t, J=5.6 Hz, 1H).

### Example 166

### Preparation of (R)—N-(1-(4-amino-7-(2-fluoropyridin-4-yl)pyrrolo[2,1-f][1,2,4]triazin-5-yl)piperidin-3-yl)-5-chloro-3-(3-hydroxy-3-methylbutoxy)thiophene-2-carboxamide

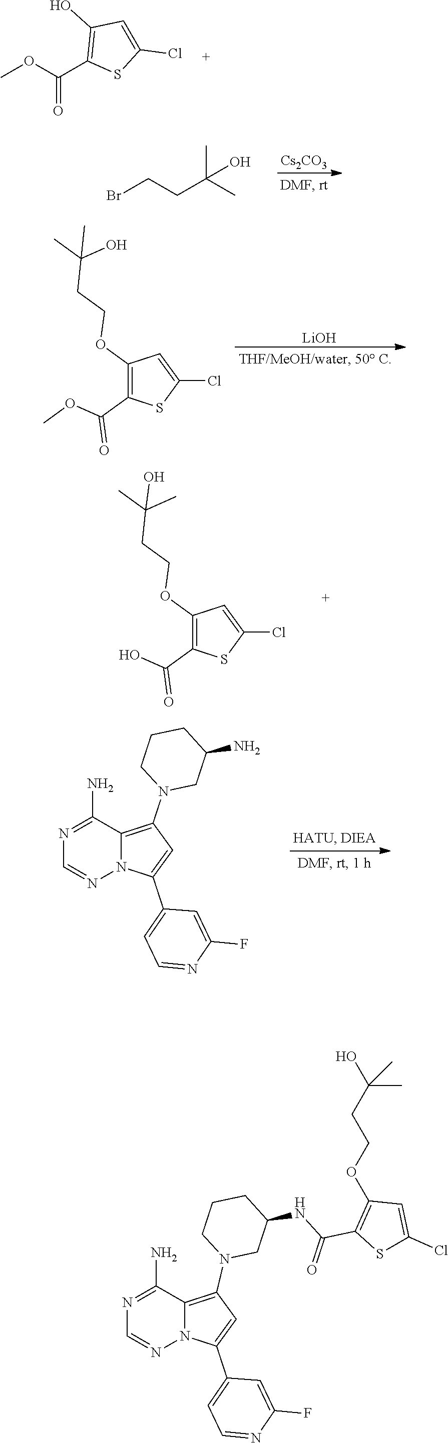

### Example 166A. Methyl 5-chloro-3-(3-hydroxy-3-methylbutoxy)thiophene-2-carboxylate

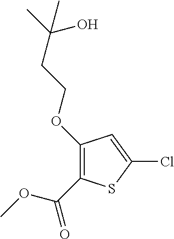 [SMILES: COC(=O)c1sc(Cl)cc1OCCC(C)(C)O]

According to the procedure for the preparation of Example 141A, reaction of methyl 5-chloro-3-hydroxythiophene-2-carboxylate and 4-bromo-2-methylbutan-2-ol afforded Example 166A as an off-white semisolid. MS: [M+H]⁺=278.90; ¹H NMR (500 MHz, CDCl₃) δ ppm 6.77 (s, 1H), 4.32 (t, J=5.9 Hz, 2H), 3.81 (s, 3H), 2.04 (t, J=5.9 Hz, 2H), 1.34 (s, 6H).

### Example 166B. 5-Chloro-3-(3-hydroxy-3-methylbutoxy)thiophene-2-carboxylic acid

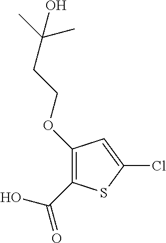 [SMILES: CC(C)(O)CCOc1cc(Cl)sc1C(=O)O]

According to the procedure for the preparation of Example 141B, reaction of Example 166A afforded Example 166B (146 mg, 85% yield) as a white solid. MS: [M+H]⁺=286.85.

### Example 166

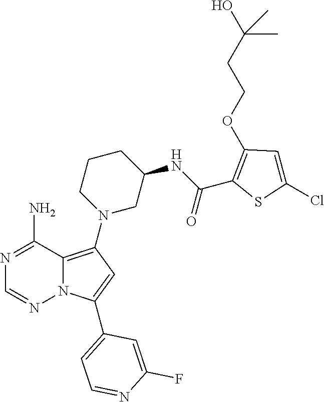 [SMILES: CC(C)(O)CCOc1cc(Cl)sc1C(=O)N[C@@H]1CCCN(c2cc(-c3ccnc(F)c3)n3ncnc(N)c23)C1]

According to the procedure for the preparation of Example 130, reaction of Example 130D and Example 166B afforded Example 166 (14.2 mg, 69% yield). LC-MS Method A: RT=2.063 min, [M+H]⁺=574.0; LC-MS Method B: RT=1.852 min, [M+H]⁺=574.0; ¹H NMR (500 MHz, DMSO-d₆) δ ppm 8.25 (d, J=5.5 Hz, 1H), 8.24-8.15 (m, 1H), 8.07 (br d, J=5.3 Hz, 1H), 8.00-7.92 (m, 2H), 7.62-7.46 (m, 1H), 7.41 (s, 1H), 7.34 (s, 1H), 6.99-6.77 (m, 1H), 4.31 (t, J=6.7 Hz, 2H), 4.21-4.09 (m, 1H), 3.47-3.36 (m, 2H), 1.93-1.75 (m, 5H), 1.10 (br s, 6H); ¹⁹F NMR (471 MHz, DMSO-d₆) δ ppm −68.78 (s, 1F).

### Example 167

### Preparation of (R)—N-(1-(4-amino-7-(2-fluoropyridin-4-yl)pyrrolo[2,1-f][1,2,4]triazin-5-yl)piperidin-3-yl)-5-chloro-3-(2-(4-methylpiperazin-1-yl)ethoxy)thiophene-2-carboxamide, 2 TFA

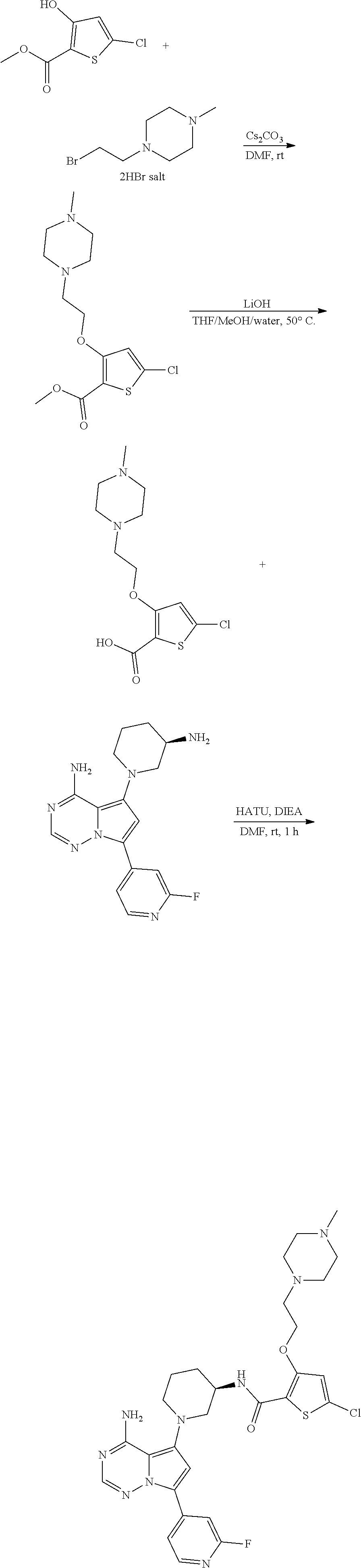

### Example 167A. Methyl 5-chloro-3-(2-(4-methylpiperazin-1-yl)ethoxy)thiophene-2-carboxylate, 2 TFA

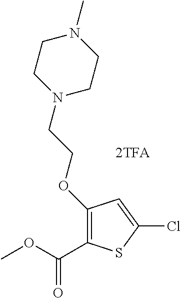 [SMILES: COC(=O)c1sc(Cl)cc1OCCN1CCN(C)CC1]

According to the procedure for the preparation of Example 141A, reaction of methyl 5-chloro-3-hydroxythiophene-2-carboxylate and 1-(2-bromoethyl)-4-methylpiperazine, dihydrobromide afforded Example 167A (43 mg, 5% yield) as an off-white semisolid. MS: [M+H]⁺=318.95; ¹H NMR (500 MHz, DMSO-d₆) δ ppm 6.72 (s, 1H), 4.63-4.59 (m, 1H), 4.50-4.46 (m, 2H), 3.79 (s, 3H), 3.72 (br d, J=0.6 Hz, 4H), 3.61 (br s, 2H), 3.56-3.52 (m, 2H), 2.94 (s, 4H).

### Example 167B. 5-Chloro-3-(2-(4-methylpiperazin-1-yl)ethoxy)thiophene-2-carboxylic acid, 2 TFA

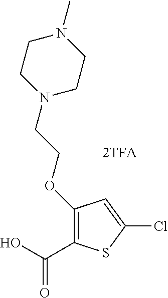 [SMILES: CN1CCN(CCOc2cc(Cl)sc2C(=O)O)CC1]

According to the procedure for the preparation of Example 141B, reaction of Example 167A afforded Example 167B (30 mg, 72% yield) as an off-white solid. MS: [M+H]⁺=304.90.

### Example 167

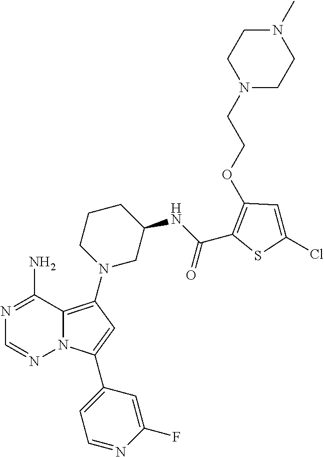 [SMILES: CN1CCN(CCOc2cc(Cl)sc2C(=O)N[C@@H]2CCCN(c3cc(-c4ccnc(F)c4)n4ncnc(N)c34)C2)CC1]

According to the procedure for the preparation of Example 130, reaction of Example 130D and Example 167B afforded Example 167 (27.7 mg, 67% yield). LC-MS Method A: RT=1.684 min, [M+H]⁺=614.2; LC-MS Method B: RT=1.283 min, [M+H]⁺=614.2; ¹H NMR (500 MHz, DMSO-d₆) δ ppm 8.26 (d, J=5.2 Hz, 1H), 8.06 (br d, J=4.6 Hz, 1H), 8.00 (s, 1H), 7.94 (s, 1H), 7.43 (s, 1H), 7.41-7.34 (m, 1H), 7.31 (s, 1H), 4.41-4.28 (m, 2H), 4.23-4.11 (m, 1H), 3.07-2.96 (m, 1H), 2.86 (br s, 2H), 2.69 (s, 3H), 2.54 (s, 9H), 1.94-1.78 (m, 3H), 1.63-1.50 (m, 1H); ¹⁹F NMR (471 MHz, DMSO-d₆) δ ppm −70.86 (s, 1F).

### Example 168

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chloro-3-(2-(4-methylpiperazin-1-yl)ethoxy)thiophene-2-carboxamide, 2 TFA

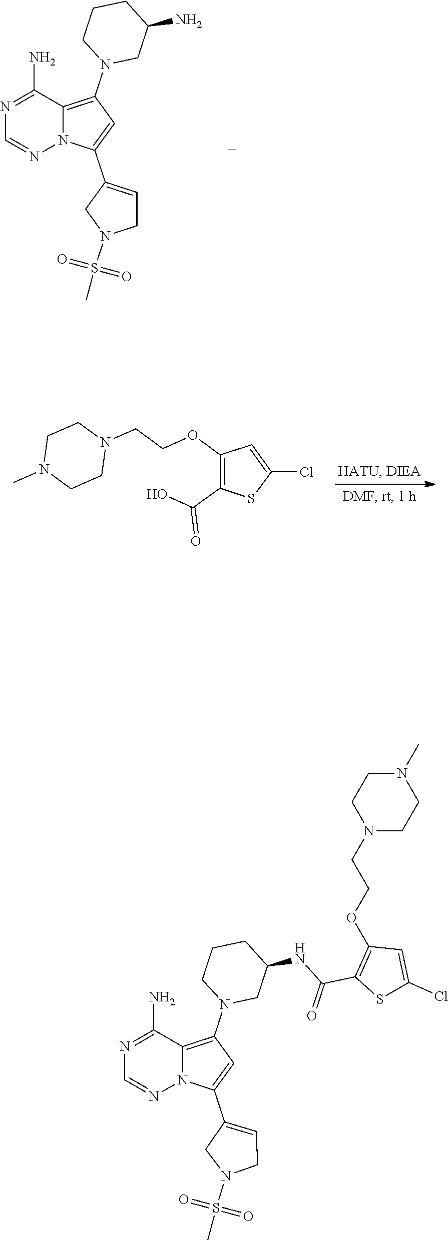 [SMILES: CS(=O)(=O)N1CC=C(c2cc(N3CCC[C@@H](N)C3)c3c(N)ncnn23)C1 ; CN1CCN(CCOc2cc(Cl)sc2C(=O)O)CC1 ; CN1CCN(CCOc2cc(Cl)sc2C(=O)N[C@@H]2CCCN(c3cc(C4=CCN(S(C)(=O)=O)C4)n4ncnc(N)c34)C2)CC1]

According to the procedure for the preparation of Example 112, reaction of Example 112G and Example 167B afforded Example 168 (5.5 mg, 39% yield). LC-MS Method A: RT=1.640 min, [M+H]⁺=664.3; LC-MS Method B: RT=1.213 min, [M+H]⁺=664.3; ¹H NMR (500 MHz, DMSO-d₆) δ ppm 7.89 (s, 1H), 7.45-7.34 (m, 1H), 7.33 (s, 1H), 7.26 (s, 1H), 7.16 (s, 1H), 7.06 (s, 1H), 6.75 (s, 1H), 6.72 (br s, 1H), 4.53 (br s, 2H), 4.41-4.27 (m, 4H), 4.21-4.07 (m, 1H), 2.95 (s, 5H), 2.81 (br s, 3H), 2.70 (br s, 3H), 2.54 (s, 3H), 2.44-2.28 (m, 1H), 1.94-1.75 (m, 3H), 1.61-1.45 (m, 1H).

### Example 169

### Preparation of (R)—N-(1-(4-amino-7-(2-fluoropyridin-4-yl)pyrrolo[2,1-f][1,2,4]triazin-5-yl)piperidin-3-yl)-5-methyl-3-vinylthiophene-2-carboxamide

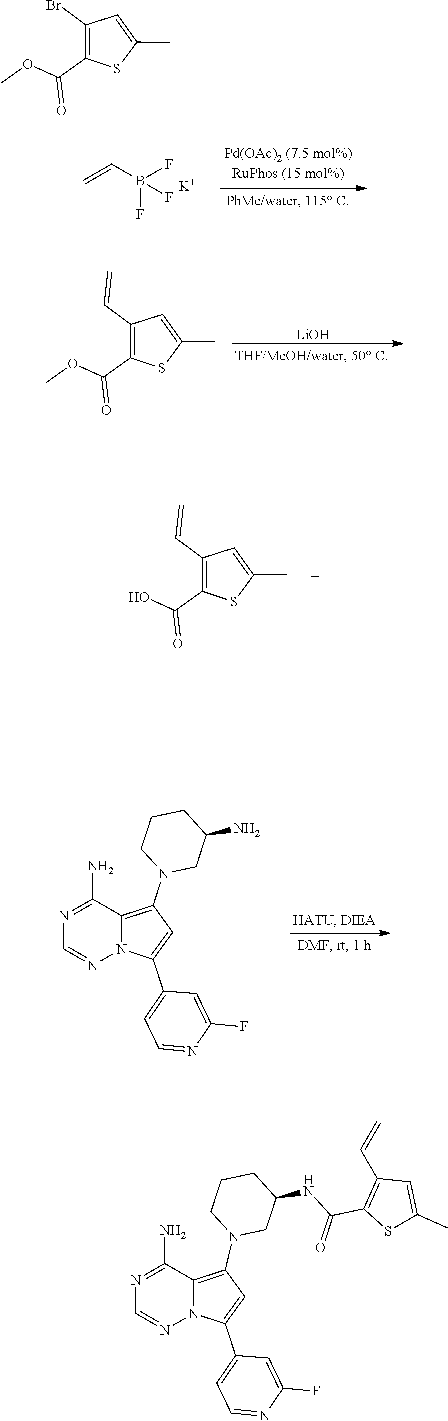

### Example 169A. Methyl 5-methyl-3-vinylthiophene-2-carboxylate

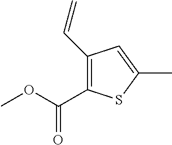

Methyl 3-bromo-5-methylthiophene-2-carboxylate (750 mg, 3.19 mmol), potassium trifluoro(vinyl)borate (641 mg, 4.79 mmol), RuPhos (223 mg, 0.479 mmol), palladium(II) acetate (53.7 mg, 0.239 mmol) and K₃PO₄ (2.03 g, 9.57 mmol) were added to a pressure vial, and the reaction mixture was degassed (3× vacuum/N₂). Then toluene (13 mL) and water (1.3 mL) were added, the reaction mixture was degassed again (3× vacuum/N₂), sealed and stirred at 115° C. for 16 h. The reaction mixture was diluted with DCM/MeOH, Celite was added, and the solvent was removed under reduced pressure. The residue was purified by flash chromatography (0-20% EtOAc/hex gradient) to give methyl Example 169A (499 mg, 86% yield) as a yellow oil, which solidified upon standing to an amber solid. MS: [M+H]⁺=183.0; ¹H NMR (500 MHz, DMSO-d₆) δ ppm 8.25 (d, J=5.5 Hz, 1H), 8.21-8.12 (m, 1H), 8.15-8.04 (m, 1H), 7.98 (s, 2H), 7.42 (s, 1H), 7.22-7.11 (m, 2H), 7.10-6.95 (m, 1H), 5.69 (dd, J=17.7, 1.0 Hz, 1H), 5.31-5.21 (m, 1H), 4.22-4.04 (m, 1H), 3.26-3.16 (m, 1H), 3.04-2.91 (m, 1H), 2.80-2.62 (m, 1H), 2.43 (s, 3H), 1.95-1.81 (m, 1H), 1.83-1.68 (m, 1H), 1.62-1.45 (m, 1H).

### Example 169B. 5-Methyl-3-vinylthiophene-2-carboxylic acid

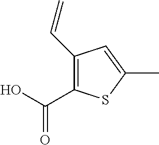

According to the procedure for the preparation of Example 141B, reaction of Example 169A afforded Example 169B (0.461 g, 100% yield) as a white solid. MS: [M+H]⁺=169.0; ¹H NMR (500 MHz, DMSO-d₆) δ ppm 7.56 (dd, J=17.9, 11.0 Hz, 1H), 7.05 (s, 1H), 5.72 (dd, J=17.7, 1.4 Hz, 1H), 5.43 (dd, J=11.0, 1.2 Hz, 1H), 2.50 (d, J=0.9 Hz, 3H).

### Example 169

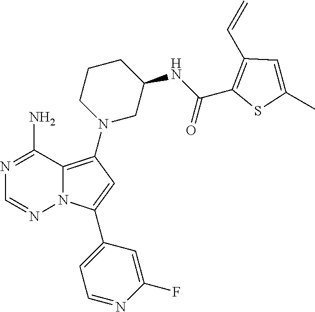 [SMILES: C=Cc1cc(C)sc1C(=O)N[C@@H]1CCCN(c2cc(-c3ccnc(F)c3)n3ncnc(N)c23)C1]

According to the procedure for the preparation of Example 130, reaction of Example 130D and Example 169B afforded Example 169 (9.3 mg, 54% yield). LC-MS Method A: RT=1.962 min, [M+H]⁺=478.0; LC-MS Method B: RT=1.701 min, [M+H]⁺=478.0; ¹H NMR (500 MHz, DMSO-d₆) δ ppm 8.25 (d, J=5.5 Hz, 1H), 8.21-8.12 (m, 1H), 8.15-8.04 (m, 1H), 7.98 (s, 2H), 7.42 (s, 1H), 7.22-7.11 (m, 2H), 7.10-6.95 (m, 1H), 5.69 (dd, J=17.7, 1.0 Hz, 1H), 5.31-5.21 (m, 1H), 4.22-4.04 (m, 1H), 3.26-3.16 (m, 1H), 3.04-2.91 (m, 1H), 2.80-2.62 (m, 1H), 2.43 (s, 3H), 1.95-1.81 (m, 1H), 1.83-1.68 (m, 1H), 1.62-1.45 (m, 1H).

### Example 170

### Preparation of (R)—N-(1-(4-amino-7-(2-fluoropyridin-4-yl)pyrrolo[2,1-f][1,2,4]triazin-5-yl)piperidin-3-yl)-3-bromo-5-methylthiophene-2-carboxamide

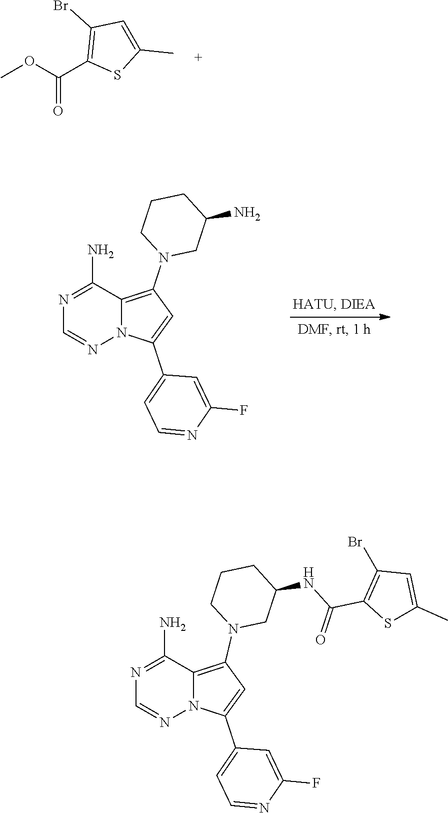

According to the procedure for the preparation of Example 130, reaction of Example 130D and 3-bromo-5-methylthiophene-2-carboxylic acid afforded Example 170 (5.6 mg, 37% yield). LC-MS Method A: RT=2.068 min, [M+H]⁺=530.0; LC-MS Method B: RT=1.721 min, [M+H]⁺=530.0; ¹H NMR (500 MHz, DMSO-d₆) δ ppm 8.30 (d, J=5.5 Hz, 1H), 8.27-8.19 (m, 1H), 8.14 (br d, J=5.2 Hz, 1H), 8.06 (br d, J=6.1 Hz, 1H), 8.03 (s, 2H), 7.48 (s, 1H), 7.13-6.99 (m, 1H), 6.96 (s, 1H), 4.25-4.14 (m, 1H), 3.14-2.69 (m, 2H), 2.59 (s, 3H), 2.00-1.88 (m, 2H), 1.88-1.77 (m, 1H), 1.71-1.56 (m, 1H).

### Example 171

### Preparation of (R)—N-(1-(4-amino-7-(2,5-dihydro-1H-pyrrol-3-yl)pyrrolo[2,1-f][1,2,4]triazin-5-yl)piperidin-3-yl)-5-chloro-3-(2-(dimethylamino)ethoxy)thiophene-2-carboxamide, 3 TFA

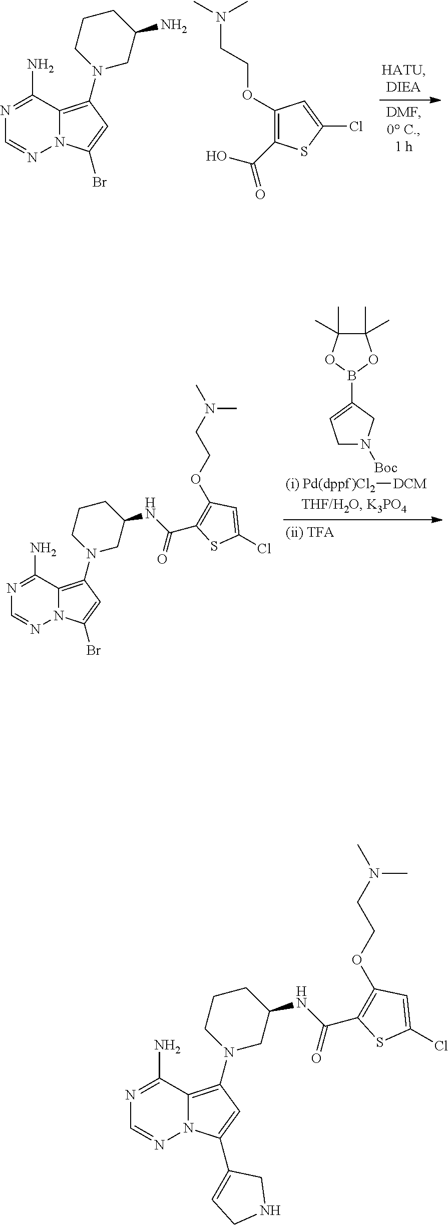

### Example 171A. (R)—N-(1-(4-Amino-7-bromopyrrolo[2,1-f][1,2,4]triazin-5-yl)piperidin-3-yl)-5-chloro-3-(2-(dimethylamino)ethoxy)thiophene-2-carboxamide, 2 TFA

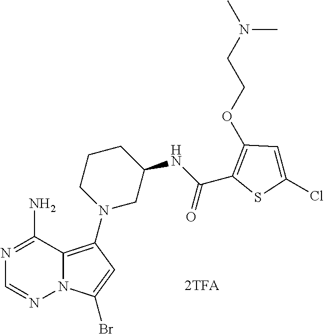 [SMILES: CN(C)CCOc1cc(Cl)sc1C(=O)N[C@@H]1CCCN(c2cc(Br)n3ncnc(N)c23)C1]

According to the procedure for the preparation of Example 112, reaction of Intermediate 61 and Example 141B at 0° C. afforded Example 171A (211 mg, 79% yield) as an off-white solid. MS: [M+H]⁺=541.94; ¹H NMR (500 MHz, DMSO-d₆) δ ppm 9.93 (br s, 1H), 8.76-8.51 (m, 1H), 7.94 (s, 1H), 7.37 (s, 1H), 7.20 (br d, J=7.8 Hz, 1H), 6.81 (s, 1H), 4.56 (t, J=4.9 Hz, 2H), 4.19-4.08 (m, 1H), 3.55 (br s, 2H), 3.19 (br dd, J=10.7, 3.1 Hz, 1H), 3.07-2.98 (m, 1H), 2.86 (br s, 6H), 2.77-2.63 (m, 2H), 1.99-1.71 (m, 3H), 1.63-1.43 (m, 1H).

### Example 171

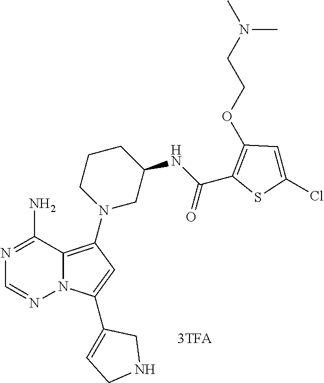 [SMILES: CN(C)CCOc1cc(Cl)sc1C(=O)N[C@@H]1CCCN(c2cc(C3=CCNC3)n3ncnc(N)c23)C1]

Example 171A (100 mg, 0.130 mmol), tert-butyl 3-(4,4,5,5-tetramethyl-1,3,2-dioxaborolan-2-yl)-2,5-dihydro-1H-pyrrole-1-carboxylate (42.1 mg, 0.143 mmol) and PdCl₂(dppf)-CH₂Cl₂ adduct (10.6 mg, 0.013 mmol) were placed in a pressure vial. Then THF (2.4 mL), water (0.3 mL) and phosphoric acid, potassium salt (110 mg, 0.519 mmol) were added, and the reaction mixture was degassed (3×, vacuum/N₂). The vial was capped, and the reaction mixture was stirred at 75° C. for 14 h. The mixture was treated with Silicycle SiliaMetS DMT scavenger silica gel, then was filtered, solid rinsed with THF, and the filtrate was concentrated. The residue was treated with TFA (2.0 mL) and stirred at rt for 30 min, TFA was removed under reduced pressure, the crude material was purified by preparative HPLC to give Example 171 (70 mg, 62% yield) as an amber glass. LC-MS Method A: RT=1.362 min, [M+H]⁺=531.2; LC-MS Method B: RT=0.922 min, [M+H]⁺=531.2; ¹H NMR (500 MHz, DMSO-d₆) δ ppm 9.55-9.41 (m, 1H), 7.92 (s, 1H), 7.51 (br d, J=7.3 Hz, 1H), 7.41 (br t, J=7.6 Hz, 1H), 7.35 (s, 1H), 7.30-7.20 (m, 1H), 6.78 (s, 1H), 6.72 (br s, 1H), 4.55 (br s, 2H), 4.39 (br s, 2H), 4.18 (br s, 2H), 4.14-4.04 (m, 1H), 3.23-3.13 (m, 1H), 3.06-2.94 (m, 1H), 2.85 (s, 6H), 2.78-2.63 (m, 2H), 1.99-1.84 (m, 1H), 1.86-1.72 (m, 2H), 1.65-1.46 (m, 1H).

### Example 172

### Preparation of (R)—N-(1-(4-amino-7-(1,2,3,6-tetrahydropyridin-4-yl)pyrrolo[2,1-f][1,2,4]triazin-5-yl)piperidin-3-yl)-5-chloro-3-(2-(dimethylamino)ethoxy)thiophene-2-carboxamide, 3 TFA

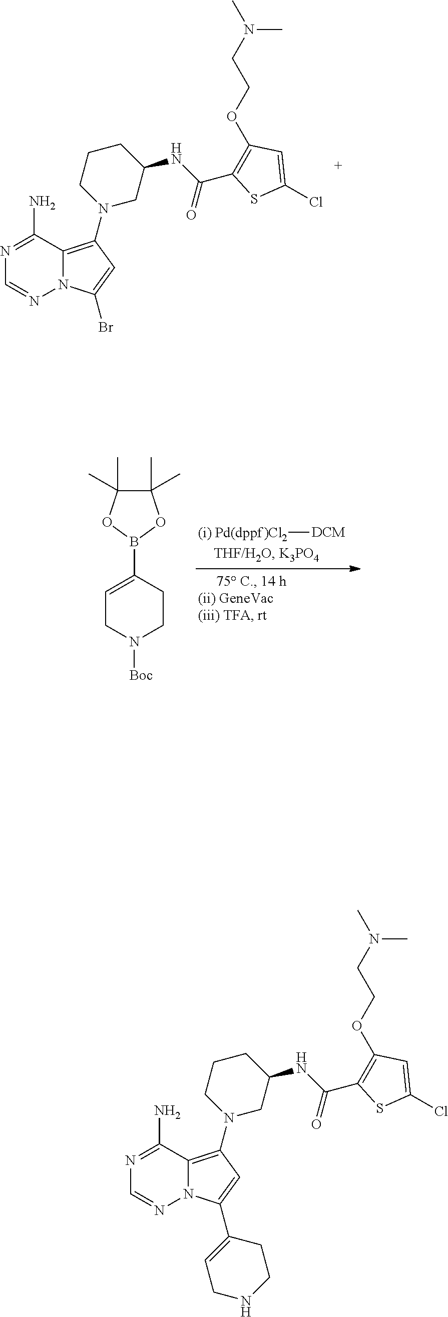

According to the procedure for the preparation of Example 171, reaction of Example 171A and tert-butyl 4-(4,4,5,5-tetramethyl-1,3,2-dioxaborolan-2-yl)-3,6-dihydropyridine-1(2H)-carboxylate afforded Example 172 (71 mg, 62% yield) as an amber glass. LC-MS Method A: RT=1.372 min, [M+H]⁺=545.2; LC-MS Method B: RT=0.942 min, [M+H]⁺=545.2; ¹H NMR (500 MHz, DMSO-d₅) δ ppm 9.08-8.93 (m, 1H), 7.88 (s, 1H), 7.36 (s, 1H), 7.23 (br d, J=5.8 Hz, 1H), 7.02 (br s, 1H), 6.71 (s, 1H), 4.55 (br d, J=4.0 Hz, 2H), 4.20-4.03 (m, 1H), 3.82 (br s, 2H), 3.60-3.49 (m, 1H), 3.37-3.28 (m, 1H), 3.23-3.13 (m, 1H), 2.99 (br d, J=4.3 Hz, 1H), 2.85 (s, 6H), 2.72 (br s, 3H), 1.95-1.85 (m, 1H), 1.84-1.73 (m, 2H), 1.63-1.48 (m, 1H).

### Example 173

### Preparation of methyl (R)-3-(4-amino-5-(3-(5-chloro-3-(2-(dimethylamino)ethoxy)thiophene-2-carboxamido)piperidin-1-yl)pyrrolo[2,1-f][1,2,4]triazin-7-yl)-2,5-dihydro-1H-pyrrole-1-carboxylate, 2 TFA

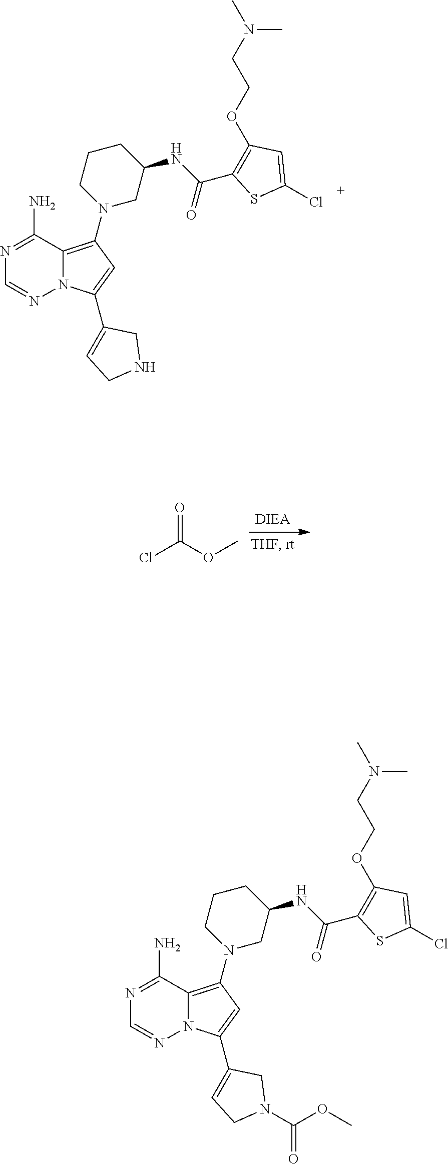

To a solution of Example 171 (15 mg, 0.017 mmol) and DIEA (0.015 mL, 0.086 mmol) in THF (1.0 mL), was added methyl chloroformate (1.7 μl, 0.022 mmol). The reaction mixture was stirred at rt for 30 min. The reaction mixture was quenched with MeOH, then was concentrated. The residue was purified by preparative HPLC to afford Example 173 (5.7 mg, 39% yield). LC-MS Method A: RT=1.612 min, [M+H]⁺=589.1; LC-MS Method B: RT=1.219 min, [M+H]⁺=589.1; ¹H NMR (500 MHz, DMSO-d₆) δ ppm 7.91 (s, 1H), 7.35 (s, 1H), 7.25-7.19 (m, 1H), 6.74 (br s, 1H), 6.73 (d, J=1.9 Hz, 1H), 4.59-4.53 (m, 3H), 4.51 (br s, 2H), 4.33-4.25 (m, 2H), 4.16-4.07 (m, 1H), 3.65 (d, J=5.4 Hz, 2H), 3.06-2.94 (m, 1H), 2.85 (s, 6H), 2.78-2.65 (m, 2H), 1.90 (br dd, J=4.9, 3.6 Hz, 1H), 1.84-1.70 (m, 2H), 1.63-1.45 (m, 1H).

### Example 174

### Preparation of methyl (R)-4-(4-amino-5-(3-(5-chloro-3-(2-(dimethylamino)ethoxy)thiophene-2-carboxamido)piperidin-1-yl)pyrrolo[2,1-f][1,2,4]triazin-7-yl)-3,6-dihydropyridine-1(2H)-carboxylate

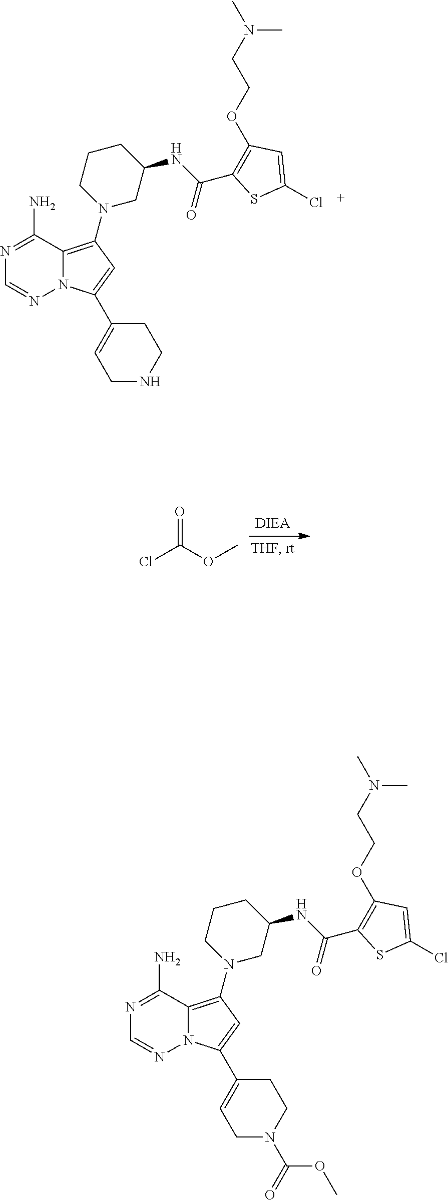

According to the procedure for the preparation of Example 173, reaction of Example 172 afforded Example 174 (4.6 mg, 45% yield). LC-MS Method A: RT=1.876 min, [M+H]⁺=603.4; LC-MS Method B: RT=1.225 min, [M+H]⁺=603.4 ¹H NMR (500 MHz, DMSO-d₆) δ ppm 7.77 (s, 1H), 7.74-7.68 (m, 1H), 7.33 (s, 1H), 6.96 (br s, 1H), 6.64 (s, 1H), 4.27 (br t, J=4.9 Hz, 2H), 4.16-4.00 (m, 3H), 3.62 (s, 2H), 3.60-3.53 (m, 1H), 2.61-2.54 (m, 6H), 2.15 (br s, 6H), 1.87 (s, 5H), 1.82-1.70 (m, 2H).

### Example 175

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-(2-(dimethylamino)ethoxy)thiophene-2-carboxamide, 2 TFA

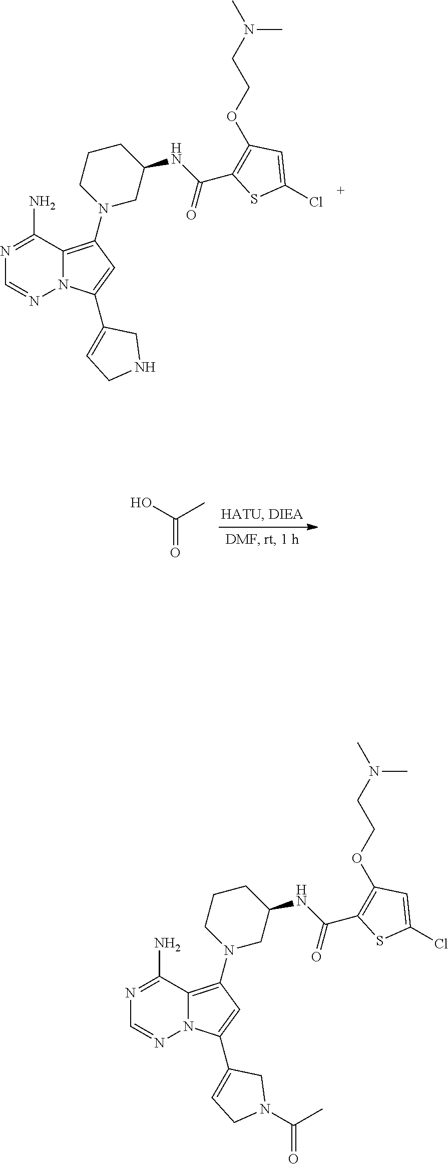

To a mixture of Example 172 (7.0 mg, 8.0 μmol), acetic acid (0.55 μl, 9.6 μmol) and DIEA (9.8 μl, 0.056 mmol) in DMF (1.5 mL), was added HATU (3.7 mg, 9.6 μmol). The reaction mixture was stirred at rt for 1 h. The reaction mixture was quenched with MeOH (0.1 mL), then was purified by preparative HPLC to afford Example 175 (3.9 mg, 59% yield). LC-MS Method A: RT=1.552 min, [M+H]⁺=573.4; LC-MS Method B: RT=1.161 min, [M+H]⁺=573.4; ¹H NMR (500 MHz, DMSO-d₆) δ ppm 7.92 (s, 1H), 7.35 (s, 1H), 7.24-7.20 (m, 1H), 6.81-6.76 (m, 1H), 6.71 (d, J=11.9 Hz, 1H), 4.68 (br s, 1H), 4.55 (br t, J=4.3 Hz, 2H), 4.51-4.42 (m, 2H), 4.27 (br s, 1H), 4.16-4.07 (m, 1H), 2.85 (s, 6H), 2.78-2.68 (m, 2H), 2.02 (d, J=19.4 Hz, 3H), 1.95-1.86 (m, 1H), 1.80 (br d, J=4.0 Hz, 2H).

The following examples in Table 7 were prepared using the same procedure as shown in Example 175. Example 172 was coupled with the appropriate carboxylic acid. Various coupling reagents could be used other than the one described, such as BOP, PyBop, EDC/HOBt or T3P.

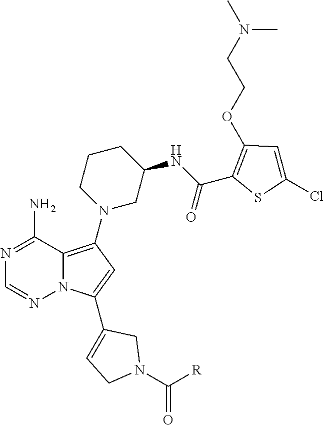

TABLE 7

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 176 | 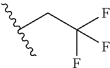 [SMILES: *CC(F)(F)F] | (R)-N-(1-(4-amino-7-(1-(3,3,3- trifluoropropanoyl)-2,5- dihydro-1H-pyrrol-3- yl)pyrrolo[2,1-f][1,2,4]triazin- 5-yl)piperidin-3-yl)-5-chloro- 3-(2- (dimethylamino)ethoxy) thiophene-2-carboxamide, 2 TFA | 641.1 | A: 1.636 B: 1.266 | (500 MHz, DMSO-d₆) δ ppm 7.91 (s, 1H), 7.35-7.30 (m, 1H), 7.24 (br t, J = 6.6 Hz, 1H), 6.81-6.76 (m, 1H), 6,75- 6.65 (m, 1H), 4.53 (br s, 4H), 4.75-4.28 (m, 2H), 4.15- 4.05 (m, 1H), 2.99 (ddd, J = 8.1, 6.3, 3.9 Hz, 1H), 2.84 (s, 6H), 2.79-2.67 (m, 2H), 1.97-1.85 (m, 1H), 1.84-1.75 (m, 2H), 1.60-1.47 (m, 1H) |
| 177 | 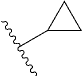 [SMILES: *C1CC1] | (R)-N-(1-(4-amino-7-(1- (cyclopropanecarbonyl)-2,5- dihydro-1H-pyrrol-3- yl)pyrrolo[2,1-f][1,2,4]triazin- 5-yl)piperidin-3-yl)-5-chloro- 3-(2- (dimethylamino)ethoxy) thiophene-2-carboxamide, 2 TFA | 598.9 | A: 1.462 B: 1.205 | (500 MHz, DMSO-d₆) δ ppm 7.92 (d, J = 2.1 Hz, 1H), 7.34 (s, 1H), 7.24-7.18 (m, 1H), 6.81 (br d, J = 8.2 Hz, 1H), 6.73 (d, J = 15.3 Hz, 1H), 4.87 (br s, 1H), 4.65 (br s, 1H), 4.56-4.47 (m, 3H), 4.29 (br s, 1H), 4.18-4.02 (m, 1H), 2.99 (br dd, J = 8.9, 2.1 Hz, 1H), 2.84 (br s, 6H), 2.77- 2.64 (m, 2H), 1.96-1.84 (m, 2H), 1.84-1.75 (m, 3H), 1.60-1.45 (m, 2H), 0.84-0.73 (m, 5H) |
| 178 | 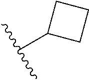 [SMILES: *C1CCC1] | (R)-N-(1-(4-amino-7-(1- (cyclobutanecarbonyl)-2,5- dihydro-1H-pyrrol-3- yl)pyrrolo[2,1-f][1,2,4]triazin- 5-yl)piperidin-3-yl)-5-chloro- 3-(2- (dimethylamino)ethoxy) thiophene-2-carboxamide, 2 TFA | 613.1 | A: 1.910 B: 1.281 | (500 MHz, DMSO-d₆) δ ppm 7.92 (s, 1H), 7.33 (d, J = 2.1 Hz, 1H), 7.26-7.20 (m, 1H), 6.78 (br d, J = 17.4 Hz, 1H), 6.72 (d, J = 4.0 Hz, 1H), 4.61-4.52 (m, 3H), 4.49 (br s, 1H), 4.37 (br s, 1H), 4.28 (br s, 1H), 4.15-4.06 (m, 1H), 3.39-3.26 (m, 1H), 3.23-3.13 (m, 1H), 3.05-2.95 (m, 1H), 2.85 (s, 6H), 2.72 (br s, 2H), 2.24-2.08 (m, 4H), 1.97-1.86 (m, 2H), 1.85-1.72 (m, 3H), 1.61-1.46 (m, 1H) |
| 179 | 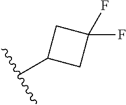 [SMILES: *C1CC(F)(F)C1] | (R)-N-(1-(4-amino-7-(1-(3,3- difluorocyclobutane-1- carbonyl)-2,5-dihydro-1H- pyrrol-3-yl)pyrrolo[2,1- f][1,2,4]triazin-5-yl)piperidin- 3-yl)-5-chloro-3-(2- (dimethylamino)ethoxy) thiophene-2-carboxamide, 2 TFA | 649.2 | A: 1.557 B: 1.267 | (500 MHz, DMSO-d₆) δ ppm 7.93 (s, 1H), 7.36 (d, J = 1.8 Hz, 1H), 7.23-7.19 (m, 1H), 6.84-6.76 (m, 1H), 6.74 (s, 1H), 4.65 (br s, 1H), 4.55 (br d, J = 3.4 Hz, 3H), 4.46 (br s, 1H), 4.33 (br s, 1H), 4.19-4.06 (m, 1H), 3.28- 3.14 (m, 2H), 3.07-2.95 (m, 1H), 2.85 (br s, 6H), 2.84- 2.76 (m, 4H), 2.76-2.66 (m, 2H), 1.95-1.87 (m, 1H), 1.81 (br s, 2H), 1.64-1.48 (m, 1H) |

### Example 180

### Preparation of (R)—N-(1-(7-(1-acetyl-1,2,3,6-tetrahydropyridin-4-yl)-4-aminopyrrolo[2,1-f][1,2,4]triazin-5-yl)piperidin-3-yl)-5-chloro-3-(2-(dimethylamino)ethoxy)thiophene-2-carboxamide 2 TFA

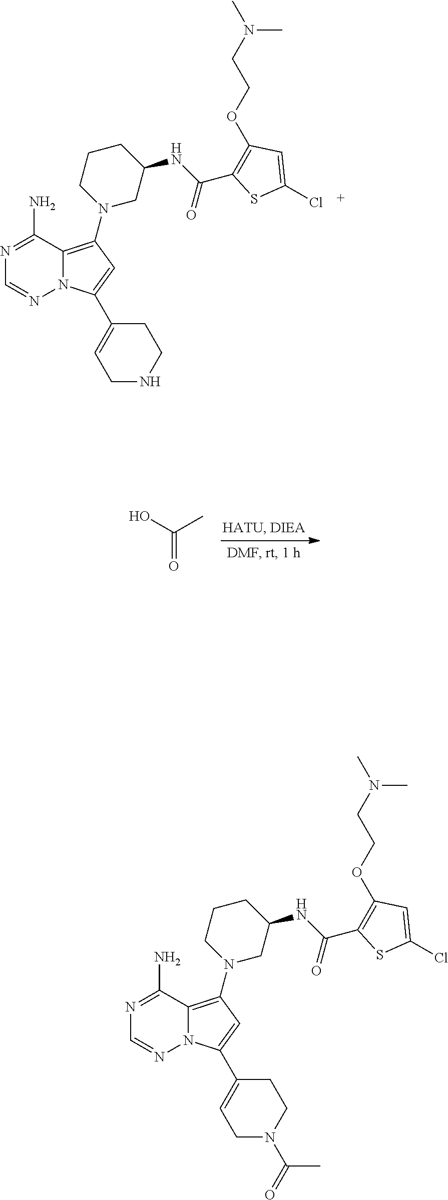

According to the procedure for the preparation of Example 175, coupling of Example 172 with acetic acid afforded Example 180 (4.8 mg, 73% yield). LC-MS Method A. RT=1.444 min, [M+H]⁺=587.4; LC-MS Method B: RT=1.116 min, [M+H]⁺=587.4; ¹H NMR (500 MHz, DMSO-d₆) δ ppm 7.89 (d, J=2.1 Hz, 1H), 7.35 (s, 1H), 7.22 (br s, 1H), 6.99-6.86 (m, 1H), 6.67 (d, J=8.2 Hz, DH), 4.55 (br t, J=4.6 Hz, 2H), 4.20 (br s, 1H), 4.13 (br s, 2H), 3.68-3.58 (m, 1H), 3.22-3.13 (m, 1H), 3.05-2.96 (m, 1H), 2.85 (s, 6H), 2.78-2.68 (m, 2H), 2.60 (br d, J=0.9 Hz, 1H), 2.04 (d, J=17.7 Hz, 3H), 1.95-1.86 (m, 1H), 1.85-1.72 (m, 2H).

The following examples in Table 8 were prepared using the same procedure as shown in Example 180. Example 172 was coupled with the appropriate carboxylic acid. Various coupling reagents could be used other than the one described, such as BOP, PyBop, EDC/HOBt or T3P.

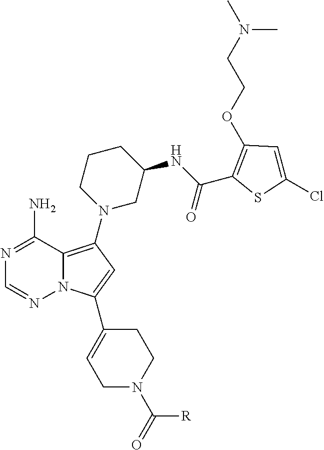

TABLE 8

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 181 | 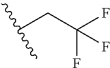 [SMILES: *CC(F)(F)F] | (R)-N-(1-(4-amino-7-(1- (3,3,3-trifluoropropanoyl)- 1,2,3,6-tetrahydropyridin-4- yl)pyrrolo[2,1- f][1,2,4]triazin-5-yl)piperidin- 3-yl)-5-chloro-3-(2- (dimethylamino)ethoxy) thiophene-2-carboxamide, 2 TFA | 654.9 | A: 1.527 B: 1.241 | (500 MHz, DMSO-d₆) δ ppm 7.87 (d, J = 3.7 Hz, 1H), 7.35 (s, 1H), 7.25-7.20 (m, 1H), 7.00-6.88 (m, 1H), 6.67 (d, J = 9.8 Hz, 1H), 4.55 (br t, J = 4.4 Hz, 2H), 4.21 (br s, 1H), 4.16 (br s, 1H), 4.14-4.06 (m, 1H), 3.78-3.60 (m, 3H), 3.23-3.14 (m, 1H), 3.04-2.97 (m, 2H), 2.85 (s, 6H), 2.73 (br d, J = 3.7 Hz, 2H), 2.66-2.59 (m, 1H), 1.95-1.85 (m, 1H), 1.85- 1.75 (m, 2H), 1.62-1.50 (m, 1H) |
| 182 | 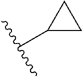 [SMILES: *C1CC1] | (R)-N-(1-(4-amino-7-(1- (cyclopropanecarbonyl)- 1,2,3,6-tetrahydropyridin-4- yl)pyrrolo[2,1- f][1,2,4]triazin-5-yl)piperidin- 3-yl)-5-chloro-3-(2- (dimethylamino)ethoxy) thiophene-2-carboxamide, 2 TFA | 612.9 | A: 1.593 B: 1.218 | (500 MHz, DMSO-d₆) δ ppm 7.89 (s, 1H), 7.36 (s, 1H), 7.24- 7.19 (m, 1H), 7.02 (br s, 1H), 6.90 (br d, J = 1.2 Hz, 1H), 6.68 (s, 1H), 4.55 (br t, J = 4.6 Hz, 2H), 4.44 (br s, 1H), 4.21- 4.08 (m, 2H), 3.89-3.83 (m, 1H), 3.67 (br d, J = 4.6 Hz, 1H), 3.22-3.13 (m, 1H), 3.06-2.97 (m, 1H), 2.85 (br s, 6H), 2.78-2.68 (m, 2H), 2.67-2.61 (m, 1H), 2.09-2.02 (m, 1H), 2.00-1.94 (m, 1H), 1.93-1.87 (m, 1H), 1.85-1.75 (m, 2H), 1.59-1.49 (m, 1H), 0.78-0.70 (m, 4H) |
| 183 | 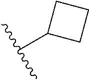 [SMILES: *C1CCC1] | (R)-N-(1-(4-amino-7-(1- (cyclobutanecarbonyl)- 1,2,3,6-tetrahydropyridin-4- yl)pyrrolo[2,1- f][1,2,4]triazin-5-yl)piperidin- 3-yl)-5-chloro-3-(2- (dimethylamino)ethoxy) thiophene-2-carboxamide, 2 TFA | 627.2 | A: 1.335 B: 1.953 | (500 MHz, DMSO-d₆) δ ppm 7.88 (s, 1H), 7.36 (s, 1H), 7.24- 7.19 (m, 1H), 6.96 (br s, 1H), 6.90 (br s, 1H), 6.66 (d, J = 6.5 Hz, 1H), 4.55 (br t, J = 4.7 Hz, 2H), 4.13 (br s, 2H), 4.10-4.05 (m, 1H), 3.89 (s, 1H), 3.58-3.52 (m, 1H), 3.23- 3.16 (m, 1H), 3.00 (br dd, J = 6.8, 3.7 Hz, 1H), 2.85 (s, 6H), 2.77-2.66 (m, 2H), 2.21-2.05 (m, 5H), 2.00-1.87 (m, 3H), 1.84-1.69 (m, 4H), 1.59-1.47 (m, 1H) |
| 184 | 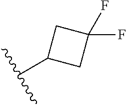 [SMILES: *C1CC(F)(F)C1] | (R)-N-(1-(4-amino-7-(1-(3,3- difluorocyclobutane-1- carbonyl)-1,2,3,6- tetrahydropyridin-4- yl)pyrrolo[2, 1- f][1,2,4]triazin-5-yl)piperidin- 3-yl)-5-chloro-3-(2- (dimethylamino)ethoxy) thiophene-2-carboxamide, 2 TFA | 663.0 | A: 1.570 B: 1.270 | (500 MHz, DMSO-d₆) δ ppm 7.87 (d, J = 2.7 Hz, 1H), 7.34 (s, 1H), 7.25-7.19 (m, 1H), 6.99-6.88 (m, 1H), 6.66 (d. J = 9.2 Hz, 1H), 4.54 (br t, J = 4.6 Hz, 2H), 4.21-4.13 (m, 2H), 4.11 (br dd, J = 7.5, 3.5 Hz, 1H), 3.74-3.64 (m, 1H), 3.62-3.50 (m, 1H), 3.37-3.23 (m, 1H), 3.00 (br dd, J = 8.1, 5.0 Hz, 1H), 2.85 (s, 6H), 2.82-2.75 (m, 4H), 2.75-2.65 (m, 2H), 2.57 (br d, J = 2.1 Hz, 1H), 1.94-1,85 (m, 1H), 1.84-1.68 (m, 2H), 1.61-1.48 (m, 1H) |

### Example 185

### Preparation of methyl (R)-3-(4-amino-5-(3-(5-chloro-3-(2-morpholinoethoxy)thiophene-2-carboxamido)piperidin-1-yl)pyrrolo[2,1-f][1,2,4]triazin-7-yl)-2,5-dihydro-1H-pyrrole-1-carboxylate, 2 TFA

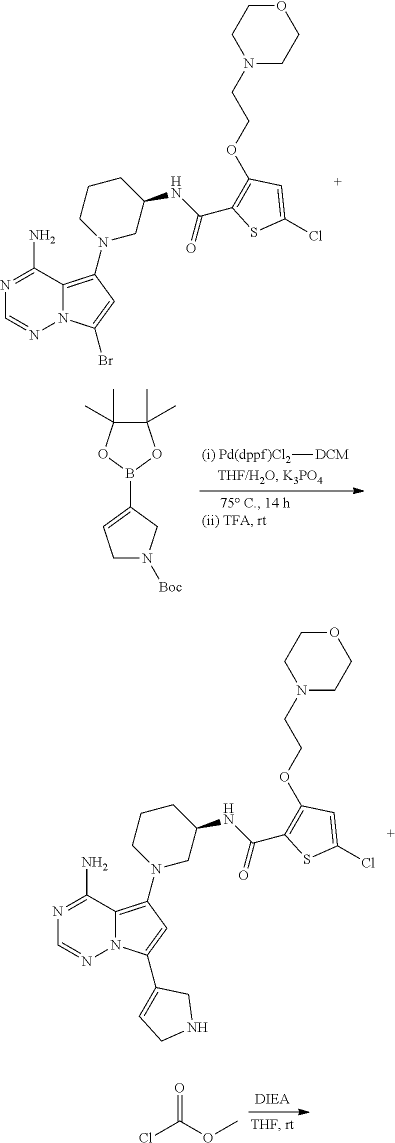

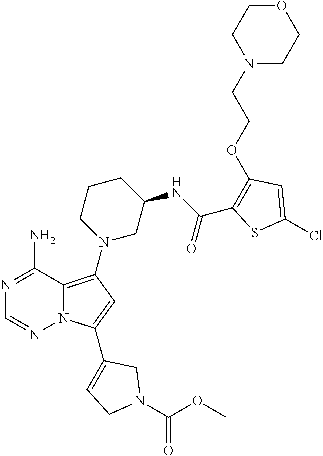 [SMILES: COC(=O)N1CC=C(c2cc(N3CCC[C@@H](NC(=O)c4sc(Cl)cc4OCCN4CCOCC4)C3)c3c(N)ncnn23)C1]

### Example 185A. (R)—N-(1-(4-amino-7-(2,5-dihydro-1H-pyrrol-3-yl)pyrrolo[2,1-f][1,2,4]triazin-5-yl)piperidin-3-yl)-5-chloro-3-(2-morpholinoethoxy)thiophene-2-carboxamide, 3TFA

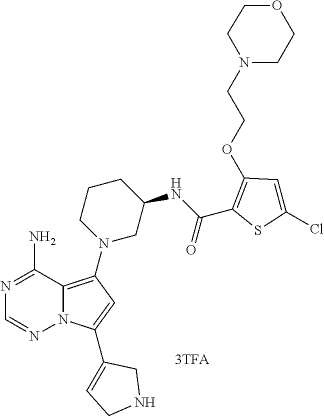 [SMILES: Nc1ncnn2c(C3=CCNC3)cc(N3CCC[C@@H](NC(=O)c4sc(Cl)cc4OCCN4CCOCC4)C3)c12]

According to the procedure for the preparation of Example 171, Example 185A was obtained as an amber glass. MS: [M+H]⁺=573.15.

### Example 185

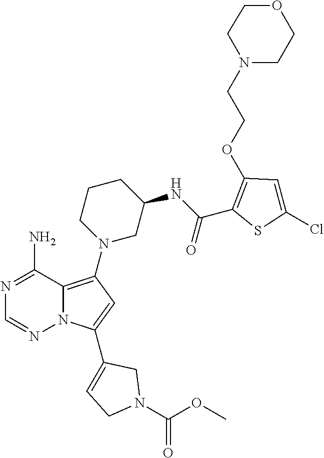 [SMILES: COC(=O)N1CC=C(c2cc(N3CCC[C@@H](NC(=O)c4sc(Cl)cc4OCCN4CCOCC4)C3)c3c(N)ncnn23)C1]

According to the procedure for the preparation of Example 173, reaction of Example 185A afforded Example 185 (15.3 mg, 70% yield) as an off-white solid. LC-MS Method A: RT=1.832 min, [M+H]⁺=631.25; LC-MS Method B: RT=1.176 min, [M+H]⁺=631.25; ¹H NMR (500 MHz, DMSO-d₆) δ ppm 7.91 (s, 1H), 7.70-7.41 (m, 1H), 7.23 (br s, 1H), 7.07 (s, 1H), 6.87 (br s, 1H), 6.72 (br d, J=12.1 Hz, 1H), 4.65-4.50 (m, 4H), 4.33 (br s, 2H), 4.29 (br d, J=3.7 Hz, 1H), 3.77 (br d, J=4.0 Hz, 4H), 3.67 (d, J=3.7 Hz, 4H), 3.27 (br d, J=9.0 Hz, 1H), 3.19 (br s, 2H), 3.09 (br d, J=9.9 Hz, 1H), 3.02-2.83 (m, 6H), 2.03-1.86 (m, 3H).

### Example 186

### (R)—N-(1-(4-amino-7-(tetrazolo[1,5-a]pyridin-7-yl)pyrrolo[2,1-f][1,2,4]triazin-5-yl)piperidin-3-yl)-5-chloro-3-(2-(dimethylamino)ethoxy)thiophene-2-carboxamide

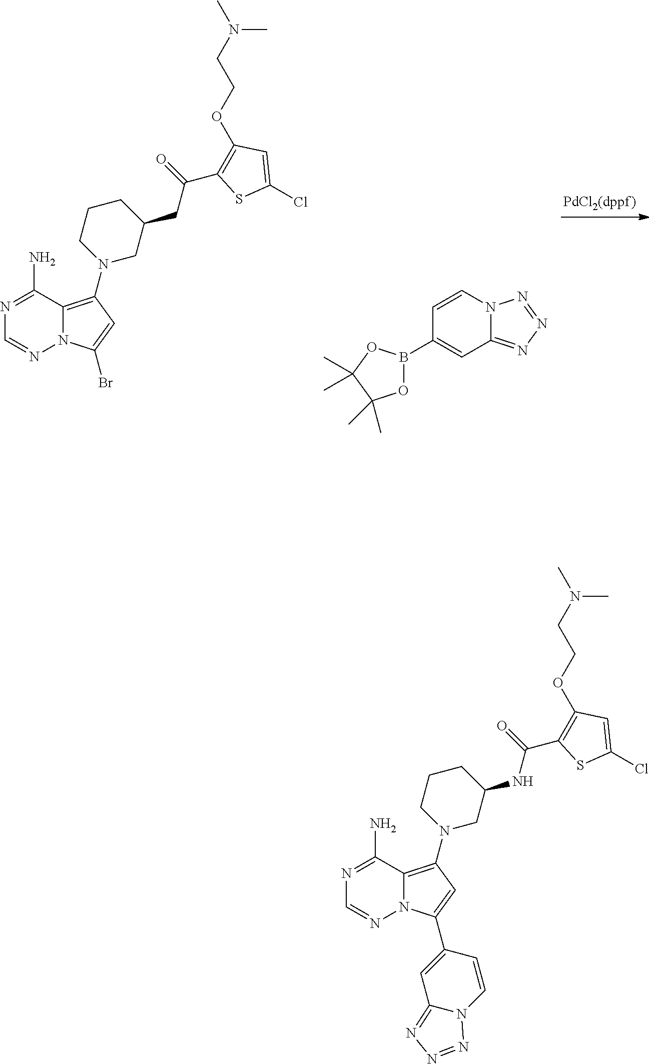 [SMILES: CN(C)CCOc1cc(Cl)sc1C(=O)C[C@@H]1CCCN(c2cc(Br)n3ncnc(N)c23)C1 ; CC1(C)OB(c2ccn3nnnc3c2)OC1(C)C ; CN(C)CCOc1cc(Cl)sc1C(=O)N[C@@H]1CCCN(c2cc(-c3ccn4nnnc4c3)n3ncnc(N)c23)C1]

To a vial containing Example 171B (20 mg, 0.037 mmol), 7-(4,4,5,5-tetramethyl-1,3,2-dioxaborolan-2-yl)tetrazolo[1,5-a]pyridine (22.2 mg, 0.044 mmol) and tripotassium phosphate (19.6 mg, 0.092 mmol), were added DMF (1 mL) and water (0.200 mL). The mixture was degassed (evacuated and flushed with N₂ (3×)), then PdCl₂(dppf)-CH₂Cl₂ adduct (6.0 mg, 7.4 μmol) was added. The mixture was degassed, then was sealed and heated at 70° C. for 1 h. The mixture was treated with ~100 mg SiliaMetS DMT. The mixture was stirred for 5 min, then was filtered rinsing with MeOH. The filtrate was purified by preparative HPLC to afford Example 186 (2.7 mg, 13% yield). LC-MS Method A: RT=1.76 min, [M+H]⁺=582.22; LC-MS Method B: RT=1.16 min, [M+H]⁺=582.33; ¹H NMR (500 MHz, DMSO-d₆) δ 9.31 (d, J=7.3 Hz, 1H), 9.10 (s, 1H), 8.24 (br s, 1H), 8.11 (dd, J=7.3, 1.2 Hz, 1H), 8.04 (s, 1H), 7.68 (br s, 1H), 7.54 (s, 1H), 7.34 (s, 1H), 6.98 (br s, 3H), 4.33 (br s, 2H), 4.18 (br s, 1H), 3.17 (br d, J=4.6 Hz, 1H), 2.74 (br s, 2H), 2.28 (br s, 3H), 1.87 (br s, 3H), 1.52 (br s, 1H)

The following examples in Table 9 were prepared using the same procedure as shown in Example 186. Example 171B was coupled with the appropriate boronic acid or boronate ester. Various coupling reagents could be used other than the one described, such as Pd(PPh₃)₄.

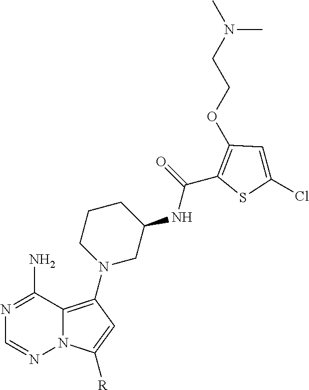

TABLE 9

| | | | | HPLC | |
|---|---|---|---|---|---|
| | | | | Method, | |
| | Structure | | LCMS | RT | |
| Example | (R) | Name | (M + H)⁺ | (min.) | ¹H NMR |
| 187 | 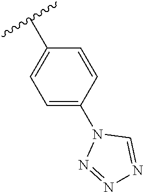 [SMILES: *c1ccc(-n2cnnn2)cc1] | (R)-N-(1-(7-(4-(1H-tetrazol- 1-yl)phenyl)-4- aminopyrrolo[2,1- f][1,2,4]triazin-5-yl)piperidin- 3-yl)-5-chloro-3-(2- (dimethylamino)ethoxy) thiophene-2-carboxamide, 2 TFA | 608.20 | A: 1.90 B: 1.23 | ¹H NMR (500 MHz, DMSO-d₆) δ 10.11 (s, 1H), 8.34 (d, J = 8.9 Hz, 2H), 7.99 (d, J = 8.9 Hz, 2H), 7.93 (s, 1H), 7.34 (s, 1H), 7.27 (s, 1H), 7.17 (s, 2H), 7.07 (s, 1H), 4.55 (br t, J = 4.3 Hz, 2H), 4.20-4.07 (m, 1H), 3.55-3.40 (m, 2H), 3.11-3.01 (m, 1H), 2.85 (s, 6H), 1.96-1.88 (m, 1H), 1.83 (br d, J = 4.0 Hz, 2H), 1.67-1.50 (m, 2H) |
| 188 | 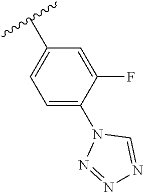 [SMILES: *c1ccc(-n2cnnn2)c(F)c1] | (R)-N-(1-(4-amino-7-(3- fluoro-4-(1H-tetrazol-1- yl)phenyl)pyrrolo[2,1- f\|[1,2,4]triazin-5-yl)piperidin- 3-yl)-5-chloro-3-(2- (dimethylamino)ethoxy) thiophene-2-carboxamide | 626.10 | A: 1.94 B: 1.26 | ¹H NMR (500 MHz, DMSO-d₆) δ 9.95 (s, 1H), 8.46- 8.38 (m, 1H), 8.23 (br d, J = 9.5 Hz, 1H), 8. 11 (br s, 1H), 7.98-7.91 (m, 2H), 7.75 (br d, J = 7.3 Hz, 1H), 7.33 (d, J = 6.1 Hz, 2H), 6.92 (br s, 1H), 4.28 (brt, J = 5.0 Hz, 2H), 4.20-4.10 (m, 1H), 2.62-2.55 (m, 2H), 2.17 (br s, 6H), 1.94-1.81 (m, 3H) |
| 189 | 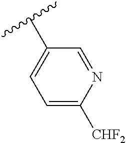 [SMILES: *c1ccc(C(F)F)nc1] | (R)-N-(1-(4-amino-7-(6- (difluoromethyl)pyridin-3- yl)pyrrolo[2,1- f][1,2,4]triazin-5-yl)piperidin- 3-yl)-5-chloro-3-(2- (dimethylamino)ethoxy) thiophene-2-carboxamide | 591.20 | A: 2.02 B: 1.27 | ¹H NMR (500 MHz, DMSO-d₆) δ 9.31 (s, 1H), 8.74- 8.66 (m, 1H), 7.90 (s, 1H), 7.80-7.68 (m, 2H), 7.34 (s, 1H), 7.28 (s, 1H), 7.10-6.82 (m, 1H), 4.28 (brt, J = 4.7 Hz, 2H), 4.17 (br s, 1H), 2.62-2.55 (m, 2H), 2.17 (br s, 6H), 1.95-1.78 (m, 4H), 1.49 (br s, 1H) |
| 190 | 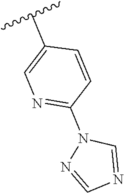 [SMILES: *c1ccc(-n2cncn2)nc1] | (R)-N-(1-(7-(6-(1H-1,2,4- triazol-1-yl)pyridin-3-yl)-4- aminopyrrolo[2,1- f][1,2,4]triazin-5-yl)piperidin- 3-yl)-5-chloro-3-(2- (dimethylamino)ethoxy) thiopbene-2-carboxamide | 608.1 | A: 1.55 B: 1.18 | ¹H NMR (500 MHz, DMSO-d₆) δ 9.38 (s, 1H), 9.19 (br d, J = 1.2 Hz, 1H), 8.75 (dd, J = 8.7, 2.0 Hz, 1H), 8.31 (s, 1H), 8.07 (br s, 1H), 7.94 (br d, J = 8.9 Hz, 1H), 7.89 (s, 1H), 7.75 (br d, J = 6.1 Hz, 1H), 7.33 (s, 1H), 7.24 (s, 1H), 6.89 (br s, 1H), 4.27 (brt, J = 4.1 Hz, 2H), 4.21-4.10 (m, 1H), 3.09-3.00 (m, 1H), 2.63-2.55 (m, 2H), 2.16 (br s, 6H), 1.98-1.76 (m, 4H) |
| 191 | 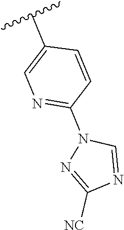 [SMILES: *c1ccc(-n2cnc(C#N)n2)nc1] | (R)-N-(1-(4-amino-7-(6-(3- cyano-1H-1,2,4-triazol-1- yl)pyridin-3-yl)pyrrolo[2,1- f][1,2,4]triazin-5-yl)piperidin- 3-yl)-5-chloro-3-(2- (dimethylamino)ethoxy) thiophene-2-carboxamide | 633.31 | A: 1.86 B: 1.36 | ¹H NMR (500 MHz, DMSO-d₆) δ 9.73 (s, 1H), 9.25 (d, J = 1.8 Hz, 1H), 8.83 (br d, J = 8.6 Hz, 1H), 8.11 (br s, 1H), 8.01 (d, J = 8.5 Hz, 1H), 7.91 (s, 1H), 7.75 (br d, J = 7.0 Hz. 1H), 7.32 (d, J = 15.9 Hz, 2H), 6.90 (br s, 1H), 4.28 (br t, J = 5.0 Hz, 2H), 4.17 (br s, 1H), 2.63-2.56 (m, 2H), 2.17 (br s, 6H), 1.97-1.75 (m, 3H), 1.51 (br s, 1H) |
| 192 | 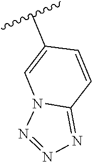 [SMILES: *c1ccc2nnnn2c1] | (R)-N-(1-(4-amino-7- (tetrazolo[1,5-a]pyridin-6- yl)pyrrolo[2,1- f][1,2,4]triazin-5-yl)piperidin- 3-yl)-5-chloro-3-(2- (dimethylamino)ethoxy) thiophene-2-carboxamide | 582.20 | A: 1.78 B: 1.14 | ¹H NMR (500 MHz, DMSO-d₆) δ 10.11 (s, 1H), 8.53- 8.46 (m, 1H), 8.28 (d, J = 9.2 Hz, 1H), 8.25-8.11 (m, 1H), 8.01 (s, 1H), 7.75 (br d, J = 6.7 Hz, 1H), 7.44 (s, 1H), 7.35 (s, 1H), 6.94 (br s, 1H), 4.29 (br t, J = 4.7 Hz, 2H), 4.19 (br s, 1H), 2.66-2.56 (m, 2H), 2.19 (br s, 6H), 2.00- 1.78 (m, 3H), 1,60-1.36 (m, 1H) |
| 193 | 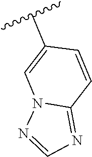 [SMILES: *c1ccc2ncnn2c1] | (R)-N-(1-(7- ([1,2,4]triazolo{ 1,5-a]pyridin- 6-yl)-4-aminopyrrolo[2,1- f][1,2,4]triazin-5-yl)piperidin- 3-yl)-5-chloro-3-(2- (dimethylamino)ethoxy) thiophene-2-carboxamide | 581.06 | A: 1.42 B: 1.10 | ¹H NMR (500 MHz, DMSO-d₆) δ 9.82 (s, 1H), 8.52 (s, 1H), 8.27 (dd, J = 9.3, 1.4 Hz, 1H), 8.09 (br s, 1H), 7.95 (s, 1H), 7.91 (d, J = 9.5 Hz, 1H), 7.74 (br d, J = 7.6 Hz, 1H), 7.34 (s, 1H), 7.31 (s, 1H), 6.89 (br s, 1H), 4.29 (br t, J = 4.4 Hz, 2H), 4.24-4.07 (m, 1H), 3.16 (br d, J = 4.3 Hz, 1H), 3.08-2.96 (m, 1H), 2.65-2.55 (m, 2H), 2.19 (br s, 6H), 1,99-1.71 (m, 3H), 1.59-1.41 (m, 1H) |

### Example 194

### N-[(3R)-1-[4-amino-6-bromo-7-(4-methanesulfonylphenyl)pyrrolo[2,1-f][1,2,4]triazin-5-yl]piperidin-3-yl]-5-methylthiophene-2-carboxamide

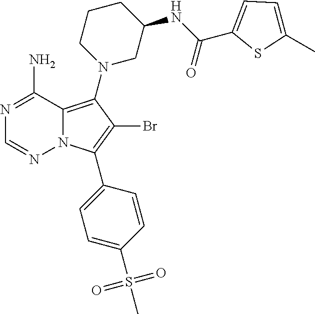 [SMILES: Cc1ccc(C(=O)N[C@@H]2CCCN(c3c(Br)c(-c4ccc(S(C)(=O)=O)cc4)n4ncnc(N)c34)C2)s1]

### Example 194A. tert-butyl (R)-(1-(4-amino-6-bromo-7-(4-(methylsulfonyl)phenyl)pyrrolo[2,1-f][1,2,4]triazin-5-yl)piperidin-3-yl)carbamate

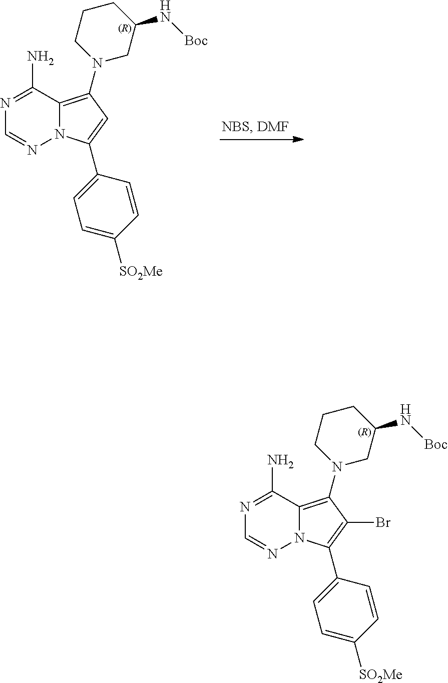

To a solution of Example 11 (200 mg, 0.411 mmol) in DCM (5 mL) at rt, was added NBS (80 mg, 0.45 mmol). The mixture was stirred at rt protected from light for 2 h. The reaction mixture was diluted with DCM, washed with water and brine solution, dried over Na₂SO₄, filtered and concentrated. The product was purified by flash chromatography (0-100% EtOAc in DCM gradient) to afford Example 194A (80 mg, 34% yield) as a yellow solid. MS: [M+H]⁺=565.0

### Example 194B. (R)-5-(3-aminopiperidin-1-yl)-6-bromo-7-(4-(methylsulfonyl)phenyl)pyrrolo[2,1-f][1,2,4]triazin-4-amine, 3 HCl

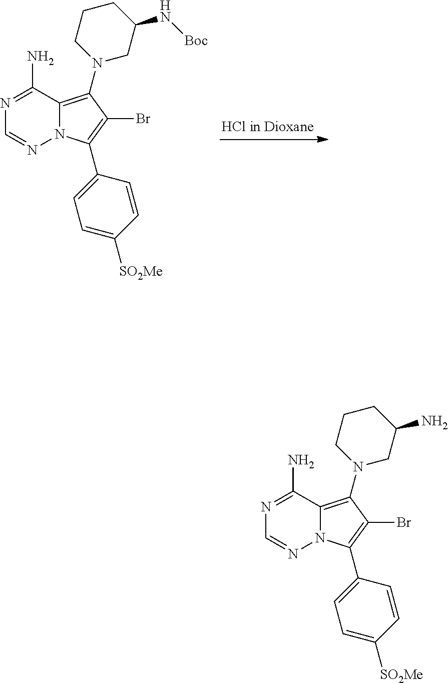 [SMILES: CC(C)(C)OC(=O)N[C@@H]1CCCN(c2c(Br)c(-c3ccc(S(C)(=O)=O)cc3)n3ncnc(N)c23)C1 ; CS(=O)(=O)c1ccc(-c2c(Br)c(N3CCC[C@@H](N)C3)c3c(N)ncnn23)cc1]

To a solution of Example 194A (80 mg, 0.141 mmol) in dioxane (1 mL), was added HCl (4 M in dioxane) (0.043 mL, 1.42 mmol). The mixture was stirred at rt for 2 h, then the reaction was concentrated, then coevaporated with toluene to afford Example 194B (85 mg, 98% yield) as a yellow solid. MS: [M+H]⁺=465.2

### Example 194

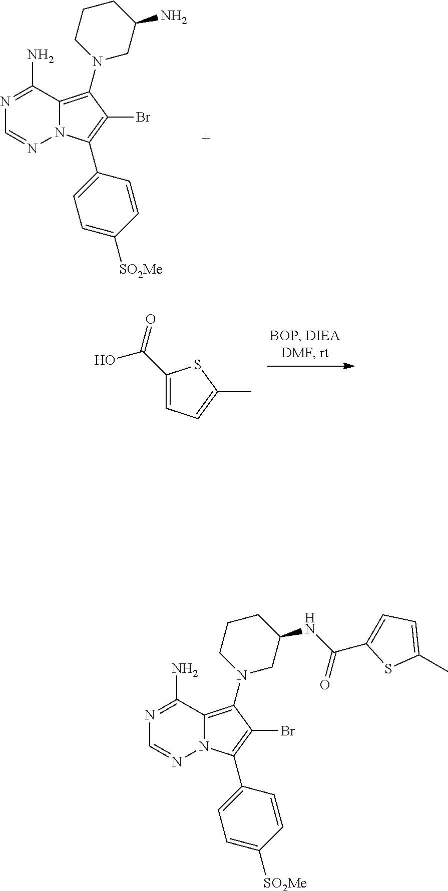

To a solution of Example 194B (86 mg, 0.141 mmol) and 5-methylthiophene-2-carboxylic acid (20 mg, 0.141 mmol) in DMF (2 mL), were added BOP (93 mg, 0.211 mmol) and DIEA (0.123 mL, 0.703 mmol). The mixture was stirred at rt for 2 h, then was quenched with ice water to afford a precipitate (80 mg) that was collected by filtration. A 15 mg aliquot was purified by preparative HPLC to afford Example 194 (8.8 mg). LC-MS Method A: RT=1.75 min, [M+H]⁺=589.1; LC-MS Method B: RT=1.51 min, [M+H]⁺=589.2; ¹H NMR (400 MHz, DMSO-d₆) δ 8.31-8.16 (m, 2H), 8.03 (d, J=8.6 Hz, 2H), 7.91 (d, J=8.6 Hz, 2H), 7.81 (s, 1H), 7.59 (d, J=3.7 Hz, 1H), 7.56-7.43 (m, 1H), 6.82 (d, J=2.9 Hz, 1H), 4.10-3.98 (m, 1H), 3.28 (s, 3H), 3.20-3.16 (m, 1H), 3.08-2.98 (m, 1H), 2.44 (s, 3H), 1.99-1.91 (m, 1H), 1.87-1.70 (m, 2H), 1.51-1.36 (m, 1H).

### Example 195

### N-[(3R)-1-[4-amino-6-chloro-7-(4-methanesulfonylphenyl)pyrrolo[2,1-f][1,2,4]triazin-5-yl]piperidin-3-yl]-5-methylthiophene-2-carboxamide

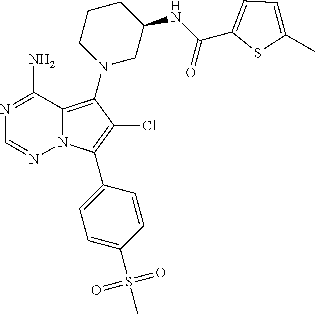 [SMILES: Cc1ccc(C(=O)N[C@@H]2CCCN(c3c(Cl)c(-c4ccc(S(C)(=O)=O)cc4)n4ncnc(N)c34)C2)s1]

According to the procedure for the preparation of Example 194, substituting NCS for NBS afforded Example 195. LC-MS Method A: RT=1.74 min, [M+H]⁺=545.1; LC-MS Method B: RT=1.52 min, [M+H]⁺=545.1; ¹H NMR (400 MHz, DMSO-d₆) δ 8.29-8.16 (m, 2H), 8.08-8.01 (m, 2H), 7.99-7.92 (m, 2H), 7.86 (s, 1H), 7.59 (d, J=3.7 Hz, 1H), 7.46 (dd, J=2.2, 1.2 Hz, 1H), 6.82 (d, J=3.2 Hz, 1H), 4.11-4.00 (m, 1H), 3.29-3.24 (m, 4H), 3.22-3.18 (m, 1H), 3.12-3.03 (m, 1H), 2.44 (s, 3H), 1.99-1.92 (m, 1H), 1.86-1.69 (m, 2H), 1.51-1.39 (m, 1H).

### Example 196

### N-[(3R)-1-[4-amino-6-chloro-7-(4-methanesulfonylphenyl)pyrrolo[2,1-f][1,2,4]triazin-5-yl]piperidin-3-yl]pyridine-4-carboxamide

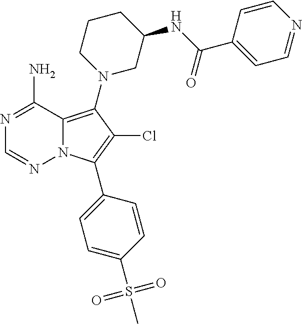 [SMILES: CS(=O)(=O)c1ccc(-c2c(Cl)c(N3CCC[C@@H](NC(=O)c4ccncc4)C3)c3c(N)ncnn23)cc1]

According to the procedure for the preparation of Example 195, amide coupling with isonicotinic acid afforded Example 196. LC-MS Method A: RT=1.41 min, [M+H]⁺=526.1; LC-MS Method B: RT=1.07 min, [M+H]⁺=526.2; ¹H NMR (400 MHz, DMSO-d₆) δ 8.76-8.68 (m, 2H), 8.68-8.58 (m, 1H), 8.26 (br s, 1H), 8.08-8.01 (m, 2H), 8.00-7.93 (m, 2H), 7.86 (s, 1H), 7.74 (d, J=5.9 Hz, 2H), 7.48 (dt, J=3.8, 1.8 Hz, 1H), 4.20-4.10 (m, 1H), 3.90 (s, 1H), 3.28 (s, 3H), 3.11-3.05 (m, 1H), 2.02-1.95 (m, 1H), 1.88-1.73 (m, 2H), 1.53-1.42 (m, 1H).

### Example 197

### N-[(3R)-1-[4-amino-6-cyano-7-(4-methanesulfonylphenyl)pyrrolo[2,1-f][1,2,4]triazin-5-yl]piperidin-3-yl]-5-methylthiophene-2-carboxamide

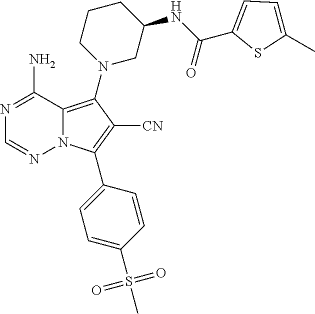 [SMILES: Cc1ccc(C(=O)N[C@@H]2CCCN(c3c(C#N)c(-c4ccc(S(C)(=O)=O)cc4)n4ncnc(N)c34)C2)s1]

To a vial containing a solution of Example 194 (40 mg, 0.068 mmol) and 1,1′-bis(diphenylphosphino)ferrocene (18.8 mg, 0.034 mmol) in NMP (2 mL), was added zinc cyanide (15.9 mg, 0.136 mmol). The mixture was bubbled with argon for 3 min, then Pd₂(dba)₃ (18.6 mg, 0.020 mmol) was added. The vial was sealed and heated at 120° C. for 16 h. The reaction was diluted with ethyl acetate. The organic layer was washed with water and brine, dried over Na₂SO₄, filtered and concentrated. The product was purified by preparative HPLC to afford Example 197 (7 mg, 19% yield). MS: [M+H]⁺=536.2; ¹H NMR (400 MHz, DMSO-d₆) δ ppm 8.60 (br s, 1H), 8.30 (d, J=8.0 Hz, 1H), 8.16-8.01 (m, 4H), 7.96 (s, 1H), 7.61 (br s, 1H), 7.44 (br s, 1H), 6.83 (dd, J=3.5, 1.0 Hz, 1H), 4.24-4.09 (m, 1H), 3.30 (s, 3H), 3.23-3.05 (m, 3H), 2.45 (s, 3H), 2.02-1.80 (m, 3H), 1.63-1.36 (m, 2H).

### Example 198

### (R)—N-(1-(4-amino-7-(2-fluoropyridin-4-yl)pyrrolo[2,1-f][1,2,4]triazin-5-yl)piperidin-3-yl)-5-methyl-3-morpholinothiophene-2-carboxamide, 2 TFA

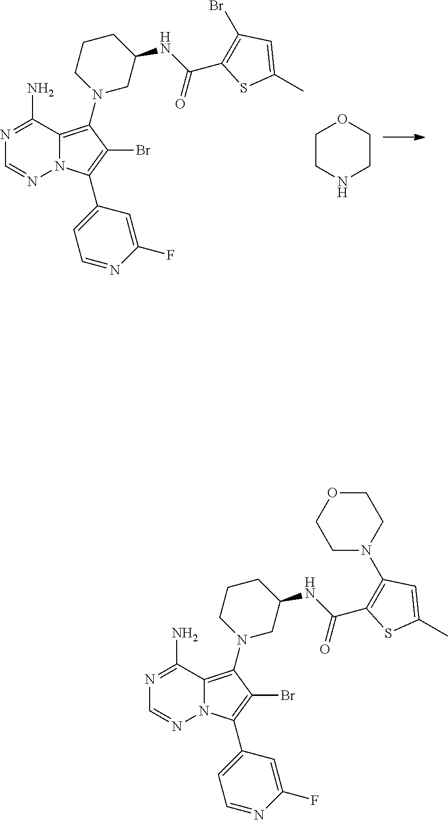 [SMILES: Cc1cc(Br)c(C(=O)N[C@@H]2CCCN(c3c(Br)c(-c4ccnc(F)c4)n4ncnc(N)c34)C2)s1 ; C1COCCN1 ; Cc1cc(N2CCOCC2)c(C(=O)N[C@@H]2CCCN(c3c(Br)c(-c4ccnc(F)c4)n4ncnc(N)c34)C2)s1]

Example 170 (25 mg, 0.047 mmol), Ir[dF(CF₃)ppy]₂(dtbbpy)PF₆ (1.1 mg, 0.94 μmol), morpholine (0.016 mL, 0.19 mmol) and DABCO (19 mg, 0.17 mmol) were placed in a pressure relief vial. The reaction mixture was degassed (3× vacuum/nitrogen), then a solution of NiBr₂-DME (2.9 mg, 9.4 μmol) in DMA (1.5 mL) was added. The reaction mixture was degassed again, capped and stirred under purple LED irradiation with fan cooling at rt for 2 d. SiliMetS DMT scavenger was added (25 mg; 0.64 mmol/g loading), the mixture was stirred for 15 min at rt, filtered. The filtrate was purified by preparative HPLC to afford Example 198 (2.3 mg, 6% yield). LC-MS Method A: RT=1.646 min, [M+H]⁺=537.2; LC-MS Method B: RT=1.694 min, [M+H]⁺=537.2. ¹H NMR (500 MHz, DMSO-d₆) δ ppm 8.88 (br d, J=7.6 Hz, 1H), 8.38-8.25 (m, 1H), 8.22 (d, J=5.2 Hz, 1H), 8.04 (br d, J=5.2 Hz, 1H), 7.96 (s, 1H), 7.92 (s, 1H), 7.41 (s, 1H), 6.99 (s, 1H), 4.18-4.05 (m, 1H), 3.70 (br s, 3H), 3.06-2.96 (m, 1H), 2.91-2.80 (m, 4H), 2.79-2.63 (m, 1H), 2.37 (s, 3H), 2.03-1.92 (m, 1H), 1.89-1.78 (m, 2H), 1.58-1.38 (m, 1H).

### Example 199

### (R)—N-(1-(4-amino-7-(2-fluoropyridin-4-yl)pyrrolo[2,1-f][1,2,4]triazin-5-yl)piperidin-3-yl)-5-methyl-3-(4-methylpiperazin-1-yl)thiophene-2-carboxamide, 3 TFA

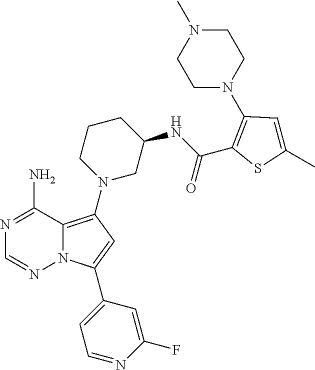 [SMILES: Cc1cc(N2CCN(C)CC2)c(C(=O)N[C@@H]2CCCN(c3cc(-c4ccnc(F)c4)n4ncnc(N)c34)C2)s1]

According to the procedure for the preparation of Example 198, coupling with 1-methylpiperazine afforded Example 199. LC-MS Method A: RT=1.838 min, [M+H]⁺=549.9; LC-MS Method B: RT=1.245 min, [M+H]⁺=549.9; ¹H NMR (500 MHz, DMSO-d₆) δ ppm 8.24 (d, J=5.4 Hz, 1H), 8.21-8.13 (m, 1H), 8.04 (br d, J=4.9 Hz, 1H), 7.97 (s, 1H), 7.93 (s, 1H), 7.39 (s, 1H), 6.93 (s, 1H), 4.17-4.06 (m, 1H), 3.74-3.56 (m, 5H), 3.33-3.22 (m, 2H), 3.09-2.99 (m, 2H), 2.97 (s, 3H), 2.90-2.71 (m, 4H), 2.40 (s, 3H), 1.94 (br d, J=9.3 Hz, 1H), 1.88-1.78 (m, 2H), 1.68-1.55 (m, 1H).

### Example 200

### (R)—N-(1-(4-amino-7-(2-fluoropyridin-4-yl)pyrrolo[2,1-f][1,2,4]triazin-5-yl)piperidin-3-yl)-5-methyl-3-((4-methylpiperazin-1-yl)methyl)thiophene-2-carboxamide

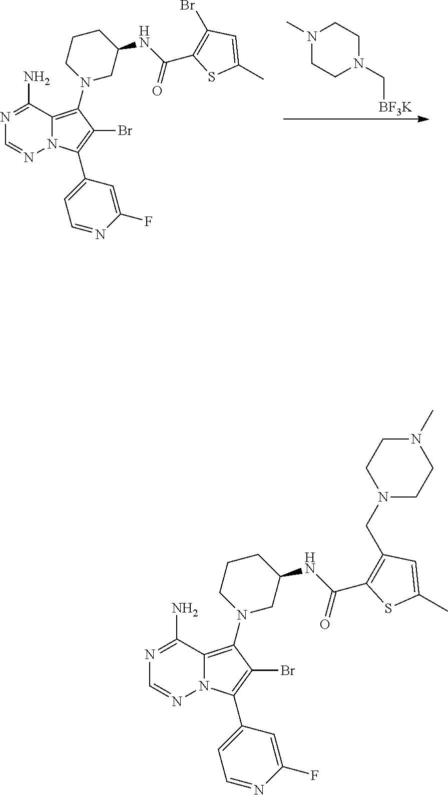

To a 8 mL pressure vial were added NiBr₂-DME (1.3 mg, 4.1 μmol), 4,4′-di-tert-butyl-2,2′-dipyridyl (1.1 mg, 4.1 μmol) and DMA (0.4 mL). The tube was sealed and the contents were stirred under N₂ for 5 min; a deep blue-green solution formed. Example 170 (18 mg, 0.034 mmol), 1-methyl-4-((trifluoro-λ⁴-boraneyl)methyl)piperazine, potassium salt (22.4 mg, 0.102 mmol), 2,6-lutidine (6.3 μl, 0.054 mmol), and Ir[dF(CF₃)ppy]₂(dtbbpy)PF₆ (1.1 mg, 1.0 μmol) were then added in succession. The contents of the reaction were stirred to dissolve as much solid as possible, then 1,4-dioxane (1.6 mL) was added. The reaction mixture was degassed (3× vacuum/N₂), capped and stirred under purple LED irradiation at rt for 3 d. SiliMetS DMT scavenger was added (25 mg; 0.64 mmol/g loading), the mixture was stirred for 15 min at rt, filtered, concentrated. The residue was purified by preparative HPLC to afford Example 200 (2.6 mg, 12% yield). LC-MS Method A: RT=1.761 min, [M+H]⁺=564.2; LC-MS Method B: RT=1.247 min, [M+H]⁺=564.2; ¹H NMR (500 MHz, DMSO-d₆) δ ppm 10.08-9.96 (m, 1H), 8.25 (d, J=5.5 Hz, 1H), 8.20-8.12 (m, 1H), 8.07 (br d, J=5.7 Hz, 1H), 8.01-7.92 (m, 2H), 7.40 (s, 1H), 7.07-6.92 (m, 1H), 6.74 (s, 1H), 4.16-4.03 (m, 1H), 3.56-3.41 (m, 3H), 3.14-3.08 (m, 1H), 2.47-2.40 (m, 3H), 2.39-2.34 (m, 1H), 2.16 (s, 3H), 2.11-2.01 (m, 1H), 1.95-1.82 (m, 2H), 1.81-1.72 (m, 2H), 1.47-1.34 (m, 1H).

### Example 201

### (R)—N-(1-(4-amino-7-(2-fluoropyridin-4-yl)pyrrolo[2,1-f][1,2,4]triazin-5-yl)piperidin-3-yl)-5-methyl-3-((2-morpholinoethoxy)methyl)thiophene-2-carboxamide, 2 TFA

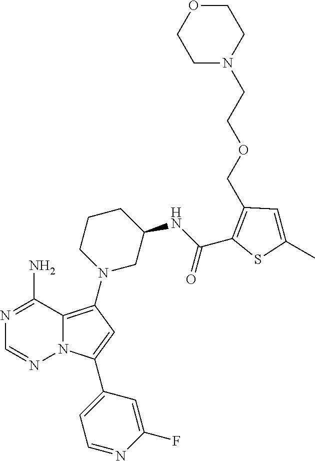 [SMILES: Cc1cc(COCCN2CCOCC2)c(C(=O)N[C@@H]2CCCN(c3cc(-c4ccnc(F)c4)n4ncnc(N)c34)C2)s1]

According to the procedure for the preparation of Example 200, coupling with 4-(2-((trifluoro-λ⁴-boraneyl)methoxy)ethyl)morpholine, potassium salt afforded Example 201. LC-MS Method A: RT=1.862 min, [M+H]⁺=595.2; LC-MS Method B: RT=1.334 min, [M+H]⁺=595.2; ¹H NMR (500 MHz, DMSO-d₆) δ ppm 8.21 (d, J=5.5 Hz, 1H), 8.03 (br d, J=5.2 Hz, 1H), 8.00-7.93 (m, 2H), 7.91 (s, 1H), 7.38 (s, 1H), 6.85 (s, 1H), 4.63 (s, 2H), 4.14-3.99 (m, 1H), 3.68 (br d, J=4.6 Hz, 1H), 3.28 (br d, J=4.6 Hz, 1H), 3.16 (br d, J=8.5 Hz, 1H), 2.96 (td, J=6.9, 5.2 Hz, 1H), 2.81-2.61 (m, 2H), 2.46 (br s, 4H), 2.40 (s, 3H), 1.92-1.79 (m, 2H), 1.78-1.67 (m, 1H), 1.56-1.43 (m, 1H).

### Example 202

### (R)—N-(1-(4-amino-7-(2-fluoropyridin-4-yl)pyrrolo[2,1-f][1,2,4]triazin-5-yl)piperidin-3-yl)-3-(hydroxymethyl)-5-methylthiophene-2-carboxamide

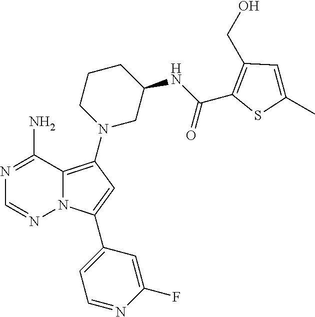 [SMILES: Cc1cc(CO)c(C(=O)N[C@@H]2CCCN(c3cc(-c4ccnc(F)c4)n4ncnc(N)c34)C2)s1]

According to the procedure for the preparation of Example 200, coupling with trimethyl(2-((trifluoro-λ⁴-boraneyl)methoxy)ethyl)silane, potassium salt afforded Example 202. LC-MS Method A: RT=1.700 min, [M+H]⁺=482.1; LC-MS Method B: RT=1.431 min, [M+H]⁺=482.1; ¹H NMR (500 MHz, DMSO-d₆) δ ppm 8.24 (d, J=5.5 Hz, 1H), 8.08 (br d, J=5.2 Hz, 1H), 7.97 (s, 2H), 7.43 (s, 1H), 7.09-6.94 (m, 1H), 6.80 (s, 1H), 4.55 (br d, J=4.3 Hz, 2H), 4.26-4.10 (m, 1H), 3.44-3.33 (m, 1H), 2.99 (s, 1H), 2.41 (s, 3H), 1.95-1.83 (m, 2H), 1.82-1.69 (m, 1H), 1.62-1.41 (m, 1H)

### Example 203

### (R)—N-(1-(4-amino-7-(3-cyclopropyl-1-methyl-1H-pyrazol-4-yl)pyrrolo[2,1-f][1,2,4]triazin-5-yl)piperidin-3-yl)-5-chlorothiophene-2-carboxamide, TFA

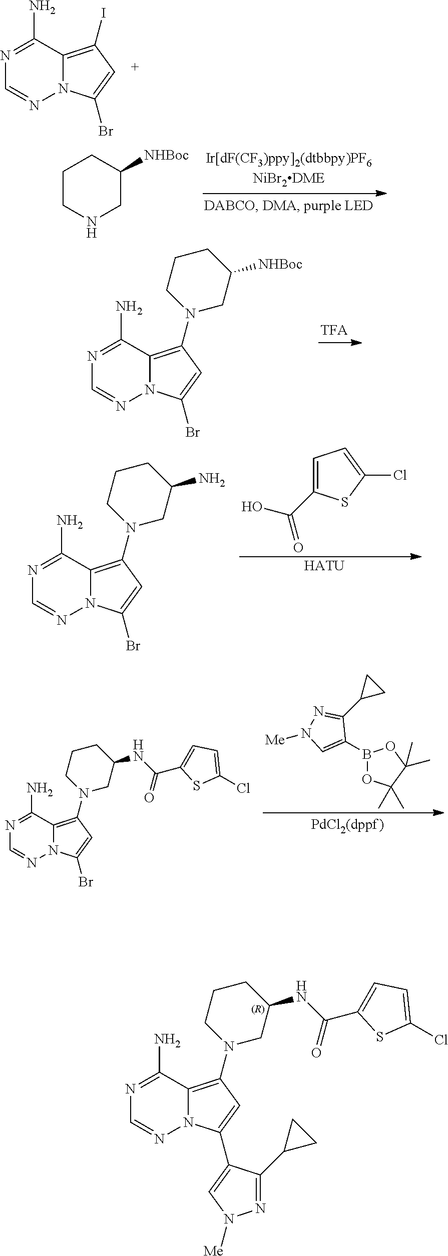

### Example 203A, Intermediate 29. tert-butyl (R)-(1-(4-amino-7-bromopyrrolo[2,1-f][1,2,4]triazin-5-yl)piperidin-3-yl)carbamate

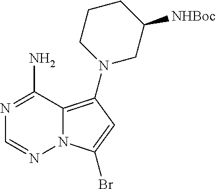 [SMILES: CC(C)(C)OC(=O)N[C@@H]1CCCN(c2cc(Br)n3ncnc(N)c23)C1]

Alternative the procedure as described in Intermediate 29, Example 203A (Intermediate 29) was prepared with a slightly modified procedure: Six vials containing 7-bromo-5-iodopyrrolo[2,1-f][1,2,4]triazin-4-amine (500 mg, 1.48 mmol), Ir[dF(CF₃)ppy]₂(dtbbpy)PF₆ (33 mg, 0.03 mmol), tert-butyl (R)-piperidin-3-ylcarbamate (1.18 g, 5.90 mmol) and DABCO (596 mg, 5.31 mmol) were degassed (3× vacuum/nitrogen), then a solution of NiBr₂-DME (91 mg, 0.30 mmol) in DMA (30 mL) was added to each vial. The reaction mixtures were degassed again, capped and stirred under purple LED irradiation with fan cooling at it for 3 days. The reactions were combined then concentrated in vacuo. The residue was dissolved in DCM and purified by flash chromatography to afford Example 203A (920 mg, 25% yield) as a yellow foam. MS: [M+H]⁺=412.80; ¹H NMR (500 MHz, CDCl₃) δ ppm 7.90 (s, 1H), 6.54 (s, 1H), 4.54 (s, 1H), 3.90-3.71 (m, 1H), 3.25 (d, J=9.3 Hz, 1H), 3.00 (s, 1H), 2.88-2.75 (m, 1H), 2.71-2.59 (m, 1H), 1.97 (dd, J=11.5, 5.4 Hz, 1H), 1.91-1.83 (m, 1H), 1.82-1.71 (m, 1H), 1.46 (s, 9H)

### Example 203B, Intermediate 61 (R)-5-(3-aminopiperidin-1-yl)-7-bromopyrrolo[2,1-f][1,2,4]triazin-4-amine, TFA salt

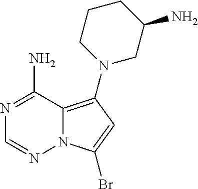 [SMILES: Nc1ncnn2c(Br)cc(N3CCC[C@@H](N)C3)c12]

MS: [M+H]⁺=310.9; ¹H NMR (500 MHz, DMSO-d₆) δ ppm 8.28-8.13 (m, 1H), 7.97 (br s, 2H), 7.88 (s, 1H), 7.10-6.96 (m, 1H), 6.83 (s, 1H), 3.55 (br s, 1H), 3.13 (br d, J=9.8 Hz, 1H), 2.95-2.82 (m, 2H), 2.78-2.65 (m, 1H), 2.07-1.89 (m, 1H), 1.89-1.76 (m, 1H), 1.75-1.54 (m, 2H).

### Example 203C. (R)—N-(1-(4-amino-7-bromopyrrolo[2,1-f][1,2,4]triazin-5-yl)piperidin-3-yl)-5-chlorothiophene-2-carboxamide

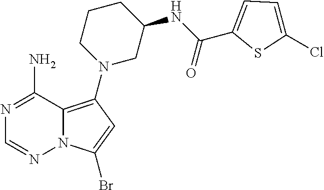 [SMILES: Nc1ncnn2c(Br)cc(N3CCC[C@@H](NC(=O)c4ccc(Cl)s4)C3)c12]

To a mixture of Intermediate 61 (640 mg, 1.19 mmol), 5-chlorothiophene-2-carboxylic acid (193 mg, 1.19 mmol) and DIEA (1.04 ml, 5.93 mmol) in DMF (11.9 ml) at 0° C., was added HATU (496 mg, 1.31 mmol). The reaction mixture was stirred at this temperature for 2 h, then was quenched with MeOH and concentrated. The resulting residue was purified by flash chromatography to afford Example 203C (454 mg, 84% yield). MS: [M+H]⁺=454.95; ¹H NMR (500 MHz, DMSO-d₆) δ ppm 8.47 (d, J=7.7 Hz, 1H), 8.24 (s, 1H), 7.93 (s, 1H), 7.70 (d, J=4.0 Hz, 1H), 7.17 (d, J=4.1 Hz, 1H), 6.93 (s, 1H), 4.23-4.11 (m, 1H), 3.80 (s, 3H), 3.24 (dd, J=10.2, 1.7 Hz, 1H), 3.18-2.97 (m, 1H), 2.85-2.67 (m, 2H), 2.05-1.96 (m, 1H), 1.96-1.89 (m, 1H), 1.88-1.82 (m, 2H), 1.56-1.46 (m, 1H), 0.93-0.88 (m, 2H), 0.78 (t, J=5.5 Hz, 2H).

### Example 203

To a vial containing 3-cyclopropyl-1-methyl-4-(4,4,5,5-tetramethyl-1,3,2-dioxaborolan-2-yl)-1H-pyrazole, HCl (13.7 mg, 0.048 mmol) and potassium phosphate (18.6 mg, 0.088 mmol), were added a solution of Example 203C (20 mg, 0.044 mmol) in THF (0.9 mL) and H₂O (0.1 mL). The mixture was degassed (evacuated and flushed with N₂, 3×). PdCl₂(dppf)-CH₂Cl₂ adduct (7.2 mg, 8.8 μmol) was added, then the mixture was degassed (3×) again. The vial was sealed and heated at 75° C. for 12 h. The mixture was treated with Pd-scavenger silica gel (~100 mg) and was stirred for 10 min. The mixture was filtered, rinsing with EtOAc, and concentrated. The residue was dissolved in 1:1 TFA/DCM (1 mL) and was stirred for 30 min. The mixture was concentrated and purified by preparative HPLC to afford Example 203 (6.2 mg, 27% yield).

LC-MS Method A: RT=1.83 min, [M+H]⁺=479.3; LC-MS Method B: RT=1.70 min, [M+H]⁺=479.0; ¹H NMR (500 MHz, DMSO-d₆) δ ppm 8.47 (d, J=7.7 Hz, 1H), 8.24 (s, 1H), 7.93 (s, 1H), 7.70 (d, J=4.0 Hz, 1H), 7.17 (d, J=4.1 Hz, 1H), 6.93 (s, 1H), 4.23-4.11 (m, 1H), 3.80 (s, 3H), 3.24 (dd, J=10.2, 1.7 Hz, 1H), 3.18-2.97 (m, 1H), 2.85-2.67 (m, 2H), 2.05-1.96 (m, 1H), 1.96-1.89 (m, 1H), 1.88-1.82 (m, 2H), 1.56-1.46 (m, 1H), 0.93-0.88 (m, 2H), 0.78 (t, J=5.5 Hz, 2H).

The following examples in Table 10 were prepared using the same procedure as shown in Example 203. Example 203C was coupled with the appropriate boronic acid or boronate ester. Various coupling reagents could be used other than the one described, such as Pd(PPh₃)₄.

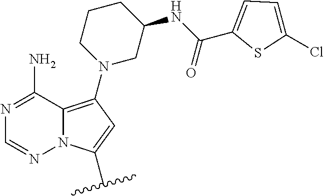 [SMILES: *c1cc(N2CCC[C@@H](NC(=O)c3ccc(Cl)s3)C2)c2c(N)ncnn12]

TABLE 10

| | | | | HPLC | |
|---|---|---|---|---|---|
| | | | | Method, | |
| | | | LCMS | RT | |
| Example | Structure | Name | (M + H)⁺ | (min.) | ¹H NMR |
| 204 | 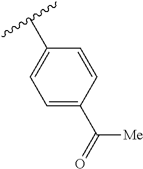 [SMILES: *c1ccc(C(C)=O)cc1] | (R)-N-(1-(7-(4- acetylphenyl)-4- aminopyrrolo[2,1- f][1,2,4]triazin-5- yl)piperidin-3-yl)-5- chlorothiophene-2- carboxamide, TFA | 495.0 | A: 2.02 B: 1.80 | (500 MHz, DMSO-d6) δ ppm 8.51 (br d, J = 7.6 Hz, 1H), 8.28 (d, J = 8.5 Hz, 2H), 8.04 (d, J = 8.5 Hz, 2H), 7.96 (s, 1H), 7.74 (d, J = 4,0 Hz, 1H), 7.23 (s, 1H), 7.20 (d, J = 4.0 Hz, 1H), 4.28-4.09 (m, 1H), 3.31-3.21 (m, 1H), 3.15-3.02 (m, 1H), 2.75 (br d, J = 8.9 Hz, 2H), 2.62 (s, 3H), 2.08-1.77 (m, 3H), 1.67-1.47 (m, 1H) |
| 205 | 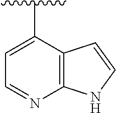 [SMILES: *c1ccnc2[nH]ccc12] | (R)-N-(1-(4-amino-7- (1H-pyrrolo[2,3- b]pyridin-4- yl)pyrrolo[2,1- f][1,2,4]triazin-5- yl)piperidin-3-yl)-5- chlorothiophene-2- carboxamide, TFA | 493.23 | A: 1.74 B: 1.42 | |
| 206 | 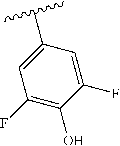 [SMILES: *c1cc(F)c(O)c(F)c1] | (R)-N-(1-(4-amino-7- (3,5-difluoro-4- hydroxyphenyl)pyrrolo [2,1-f][1,2,4]triazin-5- yl)piperidin-3-yl)-5- chlorothiophene-2- carboxamide | 505.0 | A: 1.87 B: 1.56 | (500 MHz, DMSO-d6) δ ppm 8.49 (br d, J = 7.7 Hz, 1H), 7.91-7.82 (m, 3H), 7.70 (br d, J = 4.0 Hz, 1H), 7.48-7.29 (m, 1H), 7.17 (d, J = 4.1 Hz, 1H), 7.08 (s, 1H), 4.21-4.02 (m, 1H), 3.51 (br s, 1H), 3.25-3.14 (m, 1H), 3.02 (br s, 1H), 2.75-2.64 (m, 1H), 2.00- 1.85 (m, 2H), 1.84-1.74 (m, 1H), 1.60-1.45 (m, 1H) |
| 207 | 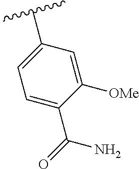 [SMILES: *c1ccc(C(N)=O)c(OC)c1] | (R)-N-(1-(4-amino-7-(4- carbamoyl-3- methoxyphenyl)pyrrolo [2,1-f][1,2,4]triazin-5- yl)piperidin-3-yl)-5- chlorothiophene-2- carboxamide | 526.3 | A: 1.60 B: 1.49 | (500 MHz, DMSO-d6) δ ppm 8.51 (d, J = 7.6 Hz, 1H), 7.93-7.86 (m, 3H), 7.84 (d, J = 7.9 Hz, 1H), 7.74 (d, J = 3.4 Hz, 1H), 7.68 (br s, 1H), 7.53 (s, 1H), 7.26-7.17 (m, 2H), 4.25-4.08 (m, 1H), 4.00 (s, 3H), 3.48-3.35 (m, 1H), 3.15-3.03 (m, 1H), 2.86-2.67 (m, 2H), 1.98- 1.81 (m, 3H), 1.64-1.45 (m, 1H) |
| 208 | 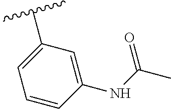 [SMILES: *c1cccc(NC(C)=O)c1] | (R)-N-(1-(7-(3- acetamidophenyl)-4- aminopyrrolo[2,1- f][1,2,4]triazin-5- yl)piperidin-3-yl)-5- chlorothiophene-2- carboxamide | 510.3 | A: 1.71 B: 1.57 | (500 MHz, DMSO-d6) δ ppm 10.04 (s, 1H), 8.50 (d, J = 7.6 Hz, 1H), 8.21 (s, 1H), 7.85 (s, 1H), 7.73 (d, J = 4.0 Hz, 1H), 7.69 (d, J = 7.6 Hz, 1H), 7.60 (d, J = 8.2 Hz, 1H), 7.37 (t, J = 7.8 Hz, 1H), 7.20 (d, J = 4.3 Hz, 1H), 6.92 (s, 1H), 4.22-4.09 (m, 1H), 3.30-3.19 (m, 1H), 3.15-3.03 (m, 1H), 2.88-2.66 (m, 2H), 2.08 (s, 3H), 2.02-1.76 (m, 3H), 1.62-1.44 (m, 1H) |
| 209 | 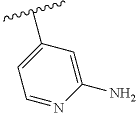 [SMILES: *c1ccnc(N)c1] | (R)-N-(1-(4-amino-7-(2- aminopyridin-4- yl)pyrrolo[2,1- f][1,2,4]triazin-5- yl)piperidin-3-yl)-5- chlorothiophene-2- carboxamide, TFA | 469.0 | A: 1.62 B: 1.30 | (500 MHz, DMSO-d6) δ ppm 8.52 (br d, J = 7.6 Hz, 1H), 8.16 (s, 1H), 8.04 (s, 1H), 7.92 (d, J = 6.7 Hz, 1H), 7.73 (br d, J = 4.0 Hz, 1H), 7.47 (s, 1H), 7.45-7.41 (m, 1H), 7.19 (d, J = 4.3 Hz, 1H), 4.15 (br s, 1H), 3.35-3.15 (m, 1H), 3.13-3.00 (m, 1H), 2.86-2.59 (m, 2H), 2.01- 1.76 (m, 3H), 1.65-1.44 (m, 1H) |
| 210 | 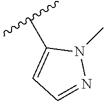 [SMILES: *c1ccnn1C] | (R)-N-(1-(4-amino-7-(1- methyl-1H-pyrazol-5- yl)pyrrolo[2,1- f][1,2,4]triazin-5- yl)piperidin-3-yl)-5- chlorothiophene-2- carboxamide, TFA | 457.18 | A: 1.79 B: 1.52 | (500 MHz, DMSO-d6) δ ppm 8.50 (br d, J = 7.6 Hz, 1H), 7.92 (s, 1H), 7.73 (d, J = 4.0 Hz, 1H), 7.54 (d. J = 1.8 Hz, 1H), 7.22-7.18 (m, 1H), 6.95 (s, 1H), 6.67 (d, J = 1.8 Hz, 1H), 4.26-4.12 (m, 1H), 3.86 (s, 3H), 3.33-3.22 (m, 1H), 3.09 (br d, J = 10.7 Hz, 1H), 2.85- 2.66 (m, 2H), 1.97-1.83 (m, 3H), 1.62-1.44 (m, 1H) |
| 211 | 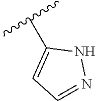 [SMILES: *c1ccn[nH]1] | (R)-N-(1-(4-amino-7- (1H-pyrazol-5- yl)pyrrolo[2,1- f][1,2,4]triazin-5- yl)piperidin-3-yl)-5- chlorothiophene-2- carboxamide | 443.30 | A: 1.58 B: 1.41 | (500 MHz, DMSO-d6) δ ppm 8.46 (d, J = 7.7 Hz, 1H), 7.90 (s, 1H), 7.80-7.65 (m, 2H), 7.18 (d, J = 4.1 Hz, 1H), 7.00 (d, J = 8.3 Hz, 2H), 4.26-4.07 (m, 1H), 3.23- 3.19 (m, 1H), 3.09-3.03 (m, 1H), 2.82-2.60 (m, 2H), 1.98-1.73 (m, 3H), 1.60-1.41 (m, 1H) |
| 212 | 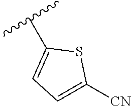 [SMILES: *c1ccc(C#N)s1] | (R)-N-(1-(4-amino-7-(5- cyanothiophen-2- yl)pyrrolo[2,1- f][1,2,4]triazin-5- yl)piperidin-3-yl)-5- chlorothiophene-2- carboxamide, TFA | 484.25 | A: 2.09 B: 1.73 | (500 MHz, DMSO-d6) δ ppm 8.48 (br d, J = 7.6 Hz, 1H), 8.06 (s, 1H), 7.96 (d, J = 4.0 Hz, 1H), 7.81 (d, J = 4.0 Hz, 1H), 7.71 (d, J = 4.0 Hz, 1H), 7.36 (s, 1H), 7.18 (d, J = 4.0 Hz, 1H), 4.28-4.06 (m, 1H), 3.31-3.17 (m, 1H), 3.13-2.99 (m, 1H), 2.78-2.60 (m, 2H), 1.97- 1.76 (m, 3H), 1.60-1.44 (m, 1H) |
| 213 | 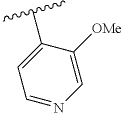 [SMILES: *c1ccncc1OC] | (R)-N-(1-(4-amino-7-(3- methoxypyridin-4- yl)pyrrolo[2,1- f][1,2,4]triazin-5- yl)piperidin-3-yl)-5- chlorothiophene-2- carboxamide, TFA | 484.20 | A: 1.81 B: 1.38 | (500 MHz, DMSO-d6) δ ppm 8.59 (br s, 1H), 8.48 (br d, J = 7.3 Hz, 1H), 8.44-8.33 (m, 2H), 7.95 (s, 1H), 7.71 (d, J = 4.0 Hz, 1H), 7.19 (s, 1H), 7.17 (d, J = 4.3 Hz, 1H), 4.23-4.08 (m, 1H), 4.01 (s, 3H), 3.26-3.16 (m, 1H), 3.11-2.98 (m, 1H), 2.82-2.65 (m, 2H), 1.95- 1.79 (m, 3H), 1.61-1.42 (m, 1H) |
| 214 | 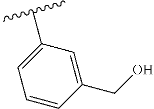 [SMILES: *c1cccc(CO)c1] | (R)-N-(1-(4-amino-7-(3- (hydroxymethyl)phenyl) pyrrolo[2,1- f][1,2,4]triazin-5- yl)piperidin-3-yl)-5- chlorothiophene-2- carboxamide | 483.09 | A: 1.58 B: 1.73 | (500 MHz, DMSO-d6) δ ppm 8.47 (br d, J = 7.3 Hz, 1H). 8.01-7.92 (m, 2H), 7.83 (s, 1H), 7.71 (d, J = 3.7 Hz, 1H), 7.39 (t, J = 7.6 Hz, 1H), 7.26 (d, J = 7.6 Hz, 1H), 7.17 (d, J = 3.7 Hz, 1H), 6.98 (s, 1H), 4.54 (s, 2H), 4.22- 4.03 (m, 1H), 3.45-3.15 (m, 1H), 3.12-2.96 (m, 1H), 2.85-2.61 (m, 2H), 1.98-1.73 (m, 3H), 1.63- 1.43 (m, 1H) |
| 215 | 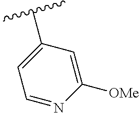 [SMILES: *c1ccnc(OC)c1] | (R)-N-(1-(4-amino-7-(2- methoxypyridin-4- yl)pyrrolo[2,1- f][1,2,4]triazin-5- yl)piperidin-3-yl)-5- chlorothiophene-2- carboxamide, TFA | 484.01 | A: 1.98 B: 1.76 | (500 MHz, DMSO-d6) δ ppm 8.48 (br d, J = 7.7 Hz, 1H), 8.18 (d, J = 5.4 Hz, 1H), 7.96 (s, 1H), 7.71 (d, J = 3.9 Hz, 1H), 7.68-7.62 (m, 1H), 7.31 (s, 1H), 7.17 (d, J = 4.0 Hz, 1H), 4.24-4.07 (m, 1H), 3.87 (m, 3H), 3.27-3.15 (m, 1H), 3.05 (m, 1H), 2.88-2.58 (m, 2H), 1.95-1.80 (m, 3H), 1.62-1.43 (m, 1H) |
| 216 | 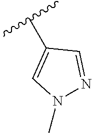 [SMILES: *c1cnn(C)c1] | (R)-N-(1-(4-amino-7-(1- methyl-1H-pyrazol-4- yl)pyrrolo[2,1- f][1,2,4]triazin-5- yl)piperidin-3-yl)-5- chlorothiophene-2- carboxamide, TFA | 457.25 | A: 1.68 B: 1.67 | (500 MHz, DMSO-d6) δ ppm 8.49 (br d, J = 7.7 Hz, 1H), 8.37 (s, 1H), 8.07 (s, 1H), 8.01 (s, 1H), 7.70 (d, J = 4.1 Hz, 1H), 7.18 (d, J = 4.1 Hz, 1H), 6.99 (s, 1H), 4.26-4.09 (m, 1H), 3.91 (s, 3H), 3.30-3.19 (m, 1H), 3.13-3.06 (m, 1H), 2.78-2.61 (m, 2H), 1.95-1.84 (m, 3H), 1.62-1.42 (m, 1H) |
| 217 | 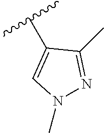 [SMILES: *c1cn(C)nc1C] | (R)-N-(1-(4-amino-7- (1,3-dimethyl-1H- pyrazol-4-yl)pyrrolo[2,1- f][1,2,4]triazin-5- yl)piperidin-3-yl)-5- chlorothiophene-2- carboxamide | 471.30 | A: 1.68 B: 1.47 | (500 MHz, DMSO-d6) δ ppm 8.46 (br d, J = 7.3 Hz, 1H), 8.25 (s, 1H), 7.85-7.73 (m, 2H), 7.69 (br d, J = 3.7 Hz, 1H), 7.16 (d, J = 4.0 Hz, 1H), 6.65 (s, 1H), 4.18- 4.03 (m, 1H), 3.81 (s, 2H), 3,23-3.14 (m, 1H), 3.06- 2.99 (m, 1H), 2.80-2.62 (m, 2H), 2.31 (s, 2H), 1.96- 1.69 (m, 3H), 1.56-1.40 (m, 1H) |
| 218 | 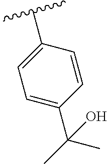 [SMILES: *c1ccc(C(C)(C)O)cc1] | (R)-N-(1-(4-amino-7-(4- (2-hydroxypropan-2- yl)phenyl)pyrrolo[2,1- f][1,2,4]triazin-5- yl)piperidin-3-yl)-5- chlorothiophene-2- carboxamide, TFA | 511.16 | A: 1.92 B: 2.26 | (500 MHz, DMSO-d6) δ ppm 8.51 (br d, J = 7.6 Hz, 1H), 8.01-7.91 (m, 3H), 7.74 (br d, J = 4.0 Hz, 1H). 7.57 (br d, J = 8.2 Hz, 2H), 7.20 (d, J = 4.0 Hz, 1H), 7.06 (s, 1H), 4.26-4.14 (m, 1H), 3.34-3.22 (m, 1H), 3.16- 3.07 (m, 1H), 2.85-2.67 (m, 2H), 1.98-1.86 (m, 3H), 1.59-1.51 (m, 1H), 1.47 (s, 6H) |
| 219 | 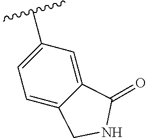 [SMILES: *c1ccc2c(c1)C(=O)NC2] | (R)-N-(1-(4-amino-7-(3- oxoisoindolin-5- yl)pyrrolo[2,1- f][1,2,4]triazin-5- yl)piperidin-3-yl)-5- chlorothiophene-2- carboxamide | 508.29 | A: 1.74 B: 1.44 | (500 MHz, DMSO-d6) δ ppm 8.69-8.51 (m, 1H), 8.46 (br s, 1H), 8.25 (d, J = 7.0 Hz, 1H), 7.92-7.85 (m, 1H), 7.72 (s, 1H), 7.67-7.59 (m, 1H), 7.24-7.09 (m, 2H), 4.40 (br s, 2H), 4.20-4.05 (m, 1H), 3.38-3.19 (m, 1H), 3.16-3.03 (m, 1H), 2.86-2.63 (m, 2H), 1,98- 1.75 (m, 3H), 1.57-1.47 (m, 1H) |
| 220 | 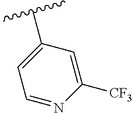 [SMILES: *c1ccnc(C(F)(F)F)c1] | (R)-N-(1-(4-amino-7-(2- (trifluoromethyl)pyridin- 4-yl)pyrrolo[2,1- f][1,2,4]triazin-5- yl)piperidin-3-yl)-5- chlorothiophene-2- carboxamide | 521.97 | A: 2.06 B: 1.82 | (500 MHz, DMSO-d6) δ ppm 8.75 (br d, J = 4.8 Hz, 1H), 8.65 (s, 1H), 8.50-8.45 (m, 2H), 8.27-8.10 (m, 1H), 7.99 (s, 1H), 7.72 (br s, 1H), 7.53 (s, 1H), 7.17 (d, J = 3.8 Hz, 1H), 7.11-6.93 (m, 1H), 4.23-4.06 (m, 1H), 2.82-2.60 (m, 2H), 2.00-1.77 (m, 4H), 1.66- 1.41 (m, 2H) |
| 221 | 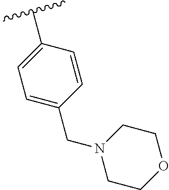 [SMILES: *c1ccc(CN2CCOCC2)cc1] | (R)-N-(1-(4-amino-7-(4- (morpholinomethyl) phenyl)pyrrolo[2,1- [][1,2,4]triazin-5- yl)piperidin-3-yl)-5- chlorothiophene-2- carboxamide, TFA | 552.14 | A: 1.99 B: 1.27 | (500 MHz, DMSO-d6) δ ppm 8.50 (br d, J = 7.6 Hz, 1H), 8.14 (d, J = 8.2 Hz, 2H), 7.98 (s, 1H), 7.72 (d, J = 4.0 Hz, 1H), 7.60 (br d, J = 8.2 Hz, 3H), 7.18 (d, J = 4.0 Hz, 1H), 7.14 (s, 1H), 4.38 (s, 2H), 4.27-4.08 (m, 1H), 3.26 (br d, J = 7.6 Hz, 2H), 3.15-3.01 (m, 2H), 2.84-2.64 (m, 2H), 2.02-1.78 (m, 4H), 1.71-1,40 (m, 2H) |
| 222 | 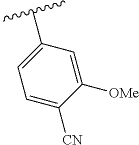 [SMILES: *c1ccc(C#N)c(OC)c1] | (R)-N-(1-(4-amino-7-(4- cyano-3- methoxyphenyl)pyrrolo [2,1-f][1,2,4]triazin-5- yl)piperidin-3-yl)-5- chlorothiophene-2- carboxamide, TFA | 508.01 | A: 2.06 B: 1.73 | (500 MHz, DMSO-d6) δ ppm 8.49 (d, J = 7.5 Hz, 1H), 7.95 (s, 1H), 7.94-7.91 (m, 1H), 7.87 (s, 1H), 7.76 (d, J = 8.1 Hz, 1H), 7.71 (d, J = 3.6 Hz, 1H), 7.32 (s, 1H), 7.17 (d, J = 4.1 Hz, 1H), 4.26-4.08 (m, 1H), 3.98 (s, 3H), 3.34-2.97 (m, 2H), 2.82-2.65 (m, 2H), 2.00- 1.78 (m, 3H), 1.61-1.44 (m, 1H) |
| 223 | 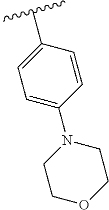 [SMILES: *c1ccc(N2CCOCC2)cc1] | (R)-N-(1-(4-amino-7-(4- morpholinophenyl)pyrrolo [2,1-f][1,2,4]triazin-5- yl)piperidin-3-yl)-5- chlorothiophene-2- carboxamide, TFA | 538.27 | A: 2.07 B: 1.66 | (500 MHz, DMSO-d6) δ ppm 8.49 (d, J = 7.9 Hz, 1H), 7.96 (s, 1H), 7.91 (d, J = 8.5 Hz, 2H), 7.71 (d, J = 4.3 Hz, 1H), 7.18 (d, J = 4.0 Hz, 1H), 7.02 (d, J = 8.9 Hz, 2H), 6.99 (s, 1H), 4.25-4.10 (m, 1H), 3.81-3.70 (m, 4H), 3.31-3.23 (m, 1H), 3.22-3.15 (m, 4H), 3.13-3.04 (m, 1H), 2.81-2.65 (m, 2H), 1.97-1.90 (m, 1H), 1.90- 1.82 (m. 2H), 1.57-1.46 (m, 1H) |
| 224 | 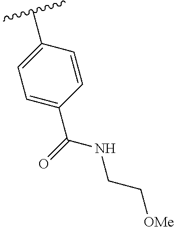 [SMILES: *c1ccc(C(=O)NCCOC)cc1] | (R)-N-(1-(4-amino-7-(4- ((2- methoxyethyl)carbamoyl) phenyl)pyrrolo[2,1- f][1,2,4]triazin-5- yl)piperidin-3-yl)-5- chlorothiophene-2- carboxamide | 554.02 | A: 1.79 B: 1.50 | |
| 225 | 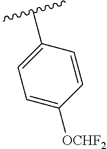 [SMILES: *c1ccc(OC(F)F)cc1] | (R)-N-(1-(4-amino-7-(4- (difluoromethoxy)phenyl) pyrrolo [2, 1- f][1,2,4]triazin-5- yl)piperidin-3-yl)-5- chlorothiophene-2- carboxamide, TFA | 519.00 | A: 2.24 B: 1.84 | (500 MHz, DMSO-d6) δ ppm 8.48 (d, J = 7.7 Hz, 1H), 8.09 (d, J = 8.7 Hz, 2H), 7.92 (s, 1H), 7.71 (d, J = 4.0 Hz, 1H), 7.31-7.25 (m, 2H), 7.18 (d, J = 4.0 Hz, 1H), 7.07 (s, 1H), 4.26-4.08 (m, 1H), 3.34-3.16 (m, 1H), 3.13- 3.01 (m, 1H), 2.87-2.63 (m, 2H), 2.01-1.76 (m, 3H), 1.62-1.42 (m, 1H) |
| 226 | 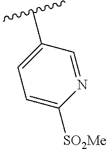 [SMILES: *c1ccc(S(C)(=O)=O)nc1] | N-[(3R)-1-[4-amino-7- (6- methanesulfonylpyridin- 3-yl)pyrrolo[2,1- f][1,2,4]triazin-5- yl]piperidin-3-yl]-5- chlorothiophene-2- carboxamide | 532.28 | A: 1.74 B: 1.49 | (500 MHz, DMSO-d6) δ ppm 9.41 (s, 1H), 8.86 (dd, J = 8.2, 2.1 Hz, 1H), 8.48 (d, J = 7.6 Hz, 1H), 8.10 (d, J = 8.5 Hz, 1H), 7.94 (s, 1H), 7.71 (d. J = 4.0 Hz, 1H), 7.35 (s, 1H), 7.17 (d, J = 4.0 Hz, 1H), 4.19-4.08 (m, 1H), 3.29 (s, 3H), 3.26-3.18 (m, 1H), 3.11-3.01 (m, 1H), 2.88-2.63 (m, 2H), 1.99-1.77 (m, 3H), 1.59- 1.46 (m, 1H) |
| 227 | 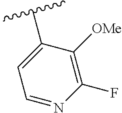 [SMILES: *c1ccnc(F)c1OC] | N-[(3R)-1-[4-amino-7- (2-fluoro-3- methoxypyridin-4- yl)pyrrolo[2,1- f][1,2,4]triazin-5- yllpiperidin-3-yl]-5- chlorothiophene-2- carboxamide, TFA | 502.26 | A: 1.89 B: 1.58 | (500 MHz, DMSO-d6) δ ppm 8.48 (d, J = 7.5 Hz, 1H), 8.02-7.92 (m, 2H), 7.87 (s, 1H), 7.70 (d, J = 3.9 Hz, 1H), 7.22-7.12 (m, 1H), 7.06 (s, 1H), 4.21-4.07 (m, 1H), 3.82 (d, J = 1.5 Hz, 2H), 3.28-3.14 (m, 1H), 3.11- 2.99 (m, 1H), 2.84-2.62 (m, 2H), 2.01-1.74 (m, 3H), 1.61-1.42 (m, 1H) |
| 228 | 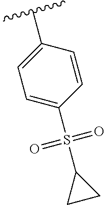 [SMILES: *c1ccc(S(=O)(=O)C2CC2)cc1] | N-[(3R)-1-{4-amino-7- [4- (cyclopropanesulfonyl)p henyl]pyrrolo[2,1- f][1,2,4]triazin-5- yl}piperidin-3-yl]-5- chlorothiophene-2- carboxamide, TFA | 556.98 | A: 1.97 B: 1.64 | (500 MHz, DMSO-d6) δ ppm 8.50 (d, J = 7.6 Hz, 1H), 8.35 (d, J = 8.9 Hz, 2H), 8.00 (s, 1H), 7.97 (d, J = 8.5 Hz, 2H), 7.74 (d, J = 4.0 Hz, 1H), 7.27 (s, 1H), 7.20 (d, J = 4.0 Hz, 1H), 4.27-4.10 (m, 1H), 3.33-3.23 (m, 1H), 3.16-3.05 (m, 1H), 2.95-2.86 (m, 1H), 2.83- 2.67 (m, 1H), 2.02-1.82 (m, 3H), 1.63-1.48 (m, 1H), 1.22-1.13 (m, 2H), 1.11-1.04 (m, 2H) |
| 229 | 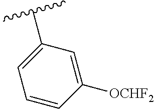 [SMILES: *c1cccc(OC(F)F)c1] | N-[(3R)-1-{4-amino-7- [3- (difluoromethoxy)phenyl Ipyrrolo [2,1- f][1,2,4]triazin-5- yl}piperidin-3-yl]-5- chlorothiophene-2- carboxamide, TFA | 519.28 | A: 2.22 B: 1.89 | (500 MHz, DMSO-d6) δ ppm 8.52 (br d, J = 7.6 Hz, 1H), 7.98 (s, 1H), 7.95 (br d, J = 7.9 Hz, 1H), 7.92 (s, 1H), 7.72 (d, J = 3.7 Hz, 1H), 7.54 (t, J = 8.1 Hz, 1H), 7.47-7.23 (m, 1H), 7.23-7.11 (m, 3H), 4.28-4.09 (m, 1H), 3.31-3.22 (m, 1H), 3.14-3.05 (m, 1H), 2.85- 2.65 (m, 2H), 2.02-1.79 (m, 3H), 1.60-1.47 (m, 1H) |
| 230 | 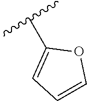 [SMILES: *c1ccco1] | N-[(3R)-1-[4-amino-7- (furan-2-yl)pyrrolo[2,1- f][1,2,4]triazin-5- yllpiperidin-3-yl]-5- chlorothiophene-2- carboxamide, TFA | 443.04 | A: 2.06 B: 1.67 | (500 MHz, DMSO-d6) δ ppm 8.49 (d, J = 7.5 Hz, 1H), 7.97 (s, 1H), 7.78 (s, 1H), 7.71 (dd, J = 4.0, 2.2 Hz, 1H), 7.18 (d, J = 2.2 Hz, 1H), 7.14 (d, J = 3.3 Hz, 1H), 6.92 (s, 1H), 6.73-6.61 (m, 1H), 4.28-4.06 (m, 1H), 3.28- 3.17 (m, 1H), 3.12-3.02 (m, 1H), 2.87-2.62 (m, 2H), 2.00-1.75 (m, 3H), 1.60-1.42 (m, 1H) |
| 231 | 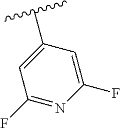 [SMILES: *c1cc(F)nc(F)c1] | N-[(3R)-1-[4-amino-7- (2,6-difluoropyridin-4- yl)pyrrolo[2,1- f][1,2,4]triazin-5- yl\|piperidin-3-yl]-5- chlorothiophene-2- carboxamide | 490.01 | A: 1.82 B: 2.17 | (500 MHz, DMSO-d6) δ ppm 8.51 (d, J = 7.6 Hz, 1H), 8.03 (s, 1H), 7.97 (s, 2H), 7.72 (d, J = 4.0 Hz, 1H), 7.54 (s, 1H), 7.19 (d, J = 4.3 Hz, 1H), 4,29-4.06 (m, 1H), 3.35-3.17 (m, 1H), 3.14-2.97 (m, 1H), 2.85-2.57 (m, 2H), 2.06-1.71 (m, 3H), 1.64-1.44 (m, 1H) |
| 232 | 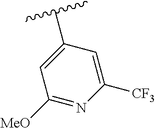 [SMILES: *c1cc(OC)nc(C(F)(F)F)c1] | N-[(3R)-1-{4-amino-7- [2-methoxy-6- (trifluoromethyl)pyridin- 4-yl]pyrrolo[2,1- f][1,2,4]triazin-5- yl}piperidin-3-yl]-5- chlorothiophene-2- carboxamide | 551.97 | A: 2.44 B: 2.10 | (500 MHz, DMSO-d6) δ ppm 8.50 (d, J = 7.6 Hz, 1H), 8.21 (s, 1H), 8.03 (s, 1H), 8.00 (s, 1H), 7.73 (d, J = 4.0 Hz, 1H), 7.55 (s, 1H), 7.19 (d, J = 4.0 Hz, 1H), 4.22- 4.07 (m, 1H), 3.95 (s, 3H), 3.31-3.16 (m, 1H), 3.13- 2.97 (m, 1H), 2.89-2.63 (m, 2H), 1.98-1.80 (m, 3H), 1.60-1.48 (m, 1H) |
| 233 | 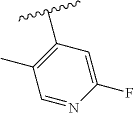 [SMILES: *c1cc(F)ncc1C] | N-[(3R)-1-[4-amino-7- (2-fluoro-5- methylpyridin-4- yl)pyrrolo[2,1- f][1,2,4]triazin-5- yllpiperidin-3-yl]-5- chlorothiophene-2- carboxamide, 2TFA | 486.31 | A: 1.98 B: 1.63 | (500 MHz, DMSO-d6) δ ppm 8.49 (d, J = 7.6 Hz, 1H), 8.22 (s, 1H), 7.88 (s, 1H), 7.73 (d, J = 3.7 Hz, 1H), 7.34 (s, 1H), 7.19 (d, J = 4.0 Hz, 1H), 6.95 (s, 1H), 4.25- 4.08 (m, 1H), 3.33-3.20 (m, 1H), 3.14-3.01 (m, 1H), 2.86-2.64 (m, 2H), 2.26 (s, 3H), 2.01-1.79 (m, 3H), 1.61-1.45 (m, 1H) |
| 234 | 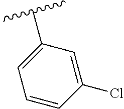 [SMILES: *c1cccc(Cl)c1] | N-[(3R)-1-[4-amino-7- (3- chlorophenyl)pyrrolo[2,1- f][1,2,4]triazin-5- yl\|piperidin-3-yl]-5- chlorothiophene-2- carboxamide | 487.07 | A: 2.34 B: 1.91 | (500 MHz, DMSO-d6) δ ppm 8.51 (d, J = 7.6 Hz, 1H), 8.24 (s, 1H), 8.04 (d, J = 8.5 Hz, 1H), 7.88 (s, 1H), 7.71 (d, J = 4.0 Hz, 1H), 7.48 (t, J = 7.8 Hz, 1H), 7.36 (d, J = 7.9 Hz, 1H), 7.18 (d, J = 4.0 Hz, 1H), 7.15 (s, 1H), 4.24-4.02 (m, 1H), 3.29-3.14 (m, 1H), 3.13-2.99 (m, 1H), 2.88-2.62 (m, 2H), 1.98-1.76 (m, 3H), 1.62- 1.43 (m, 1H) |
| 235 | 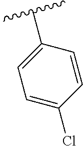 [SMILES: *c1ccc(Cl)cc1] | N-[(3R)-1-[4-amino-7- (4- chlorophenyl)pyrrolo[2,1- f][1,2,4]triazin-5- yllpiperidin-3-yl]-5- chlorothiophene-2- carboxamide | 487.09 | A: 2.31 B: 1.96 | (500 MHz, DMSO-d6) δ ppm 8.47 (d, J = 7.5 Hz, 1H), 8.13 (d, J = 8.5 Hz, 2H), 7.85 (s, 1H), 7.72 (d, J = 3.3 Hz, 1H), 7.53-7.45 (m, 2H), 7.18 (d, J = 3.9 Hz, 1H), 7.07 (s, 1H), 4.23-4.02 (m, 1H), 3.30-3.14 (m, 1H), 3.10- 2.97 (m, 1H), 2.97-2.92 (m, 1H), 2.90-2.55 (m, 2H), 1.98-1.73 (m, 3H), 1.59-1.45 (m, 1H) |
| 236 | 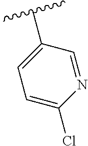 [SMILES: *c1ccc(Cl)nc1] | N-[(3R)-1-[4-amino-7- (6-chloropyridin-3- yl)pyrrolo[2,1- f][1,2,4]triazin-5- yllpiperidin-3-yl]-5- chlorothiophene-2- carboxamide | 488.15 | A: 1.68 B: 1.69 | (500 MHz, DMSO-d6) δ ppm 9.09 (d, J = 2.1 Hz, 1H), 8.59 (dd, J = 8.5, 2.4 Hz, 1H), 8.49 (d, J = 7.3 Hz, 1H), 7.90 (s, 1H), 7.73 (d, J = 3.7 Hz, 1H), 7.61 (d, J = 8.5 Hz, 1H), 7.23 (s, 1H), 7.19 (d. J = 4.0 Hz, 1H), 4.24-4.04 (m, 1H), 3.30-3.14 (m, 1H), 3.13-2.97 (m, 1H), 2.93- 2.58 (m, 2H), 2.00-1.76 (m, 3H), 1.64-1.46 (m, 1H) |
| 237 | 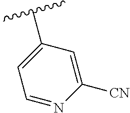 [SMILES: *c1ccnc(C#N)c1] | N-[(3R)-1-[4-amino-7- (2-cyanopyridin-4- yl)pyrrolo[2,1- f][1,2,4]triazin-5- yl]piperidin-3-yl]-5- chlorothiophene-2- carboxamide | 479.27 | A: 2.00 B: 1.60 | (500 MHz, DMSO-d6) δ ppm 9.45 (d, J = 1.2 Hz, 1H), 8.82 (dd, J = 8.4, 2.0 Hz, 1H), 8.50 (d, J = 7.6 Hz, 1H), 8.09 (d, J = 8.5 Hz, 1H), 7.96 (s, 1H), 7.74 (d, J = 3.7 Hz, 1H), 7.41 (s, 1H), 7.19 (d, J = 4.3 Hz, 1H), 4.28-4.03 (m, 1H), 3.32-3.15 (m, 1H), 3.13-2.99 (m, 1H), 2.93- 2.59 (m, 2H), 2.01-1.77 (m, 3H), 1.62-1.47 (m, 1H) |
| 238 | 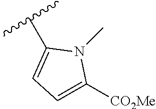 [SMILES: *c1ccc(C(=O)OC)n1C] | methyl 5-{4-amino-5- [(3R)-3-(5- chlorothiophene-2- amido)piperidin-1- yl]pyrrolo[2,1- f] [1,2,4]triazin-7-yl}-1- methyl-1H-pyrrole-2- carboxylate | 514.10 | A: 2.05 B: 1.74 | (500 MHz, DMSO-d6) δ ppm 8.49 (d, J = 7.6 Hz, 1H), 7.90 (s, 1H), 7.73 (d, J = 4.3 Hz, 1H), 7.19 (d, J = 4.0 Hz, 1H), 6.98 (d, J = 4.0 Hz, 1H), 6.88 (s, 1H), 6.47 (d, J = 4.3 Hz, 1H), 4.26-4.09 (m, 1H), 3.79 (s, 3H), 3.25 (s, 1H), 3.15-3.01 (m, 1H), 2.82-2.71 (m, 2H), 1.98- 1.94 (m, 1H), 1.91-1.84 (m, 2H), 1.55-1.50 (m, 1H) |
| 239 | 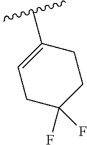 [SMILES: *C1=CCC(F)(F)CC1] | N-[(3R)-1-[4-amino-7- (4,4-difluorocyclohex-1- en-1-yl)pyrrolo[2,1- f][1,2,4]triazin-5- yllpiperidin-3-yl]-5- chlorothiophene-2- carboxamide | 493.11 | A: 2.15 B: 1.79 | (500 MHz, DMSO-d6) δ ppm 8.49 (br d, J = 7.6 Hz, 1H), 7.90 (s, 1H), 7.73 (d, J = 3.7 Hz, 1H), 7.19 (d, J = 4.0 Hz, 1H), 6.81 (br s, 1H), 6.74 (s, 1H), 4.22-4.08 (m, 1H), 3.24-3.15 (m, 1H), 3.08-2.98 (m, 1H), 2.81 (br t, J = 14.3 Hz, 2H), 2.77-2.72 (m, 2H), 2.72-2.59 (m, 2H), 2.25-2.13 (m, 2H), 1,98-1.77 (m, 3H), 1.58- 1.46 (m, 1H) |
| 240 | 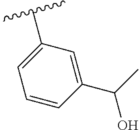 [SMILES: *c1cccc(C(C)O)c1] | N-[(3R)-1-{4-amino-7- [3-(1- hydroxyethyl)phenyl] pyrrolo[2,1- f][1,2,4]triazin- 5-yl}piperidin-3-yl]-5- chlorothiophene-2- carboxamide | 496.99 | A: 1.87 B: 1.59 | (500 MHz, DMSO-d6) δ ppm 8.48 (br d, J = 7.6 Hz, 1H), 7.96 (br s, 2H), 7.86 (s, 1H), 7.74 (d, J = 4.0 Hz, 1H), 7.46-7.36 (m, 1H), 7.31 (br d, J = 7.6 Hz, 1H), 7.20 (d, J = 4.3 Hz, 1H), 7.00 (s, 1H), 4.78 (q, J = 6.2 Hz, 1H), 4.21-4.07 (m, 1H), 3.31-3.18 (m, 1H), 3,14- 2.98 (m, 1H), 2.90-2.63 (m, 2H), 2.02-1.72 (m, 3H), 1.58-1.47 (m, 1H), 1.38 (d, J = 6.4 Hz, 3H) |
| 241 | 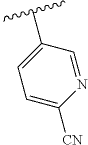 [SMILES: *c1ccc(C#N)nc1] | N-[(3R)-1-[4-amino-7- (6-cyanopyridin-3- yl)pyrrolo[2,1- f][1,2,4]triazin-5- chlorothiophene-2- carboxamide | 478.97 | A: 2.07 B: 1.60 | (500 MHz, DMSO-d6) δ ppm 9.45 (d, J = 1.2 Hz, 1H), 8.82 (dd, J = 8.4, 2.0 Hz, 1H), 8.51 (d, J = 7.9 Hz, 1H), 8.09 (d, J = 8.2 Hz, 1H), 7.96 (s, 1H), 7.73 (d, J = 4.3 Hz, 1H), 7.41 (s, 1H), 7.19 (d, J = 4.0 Hz, 1H), 4.21-4.06 (m, 1H), 3.31-3.16 (m, 1H), 3.15-2.98 (m, 1H), 2.86- 2.61 (m, 1H), 2.03-1.75 (m, 3H), 1.62-1.47 (m, 1H) |
| 242 | 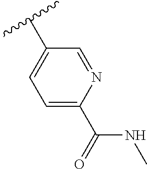 [SMILES: *c1ccc(C(=O)NC)nc1] | (R)-5-(4-amino-5-(3-(5- chlorothiophene-2- carboxamido)piperidin- 1-yl)pyrrolo[2,1- f][1,2,4]triazin-7-yl)-N- methylpicolinamide | 511.07 | A: 1.73 B: 1.47 | ¹H NMR (500 MHz, DMSO-d₆) δ 9.30-9.26 (m, 1H), 8.74 (br d, J = 4.9 Hz, 1H), 8.66 (dd, J = 8.2, 1.8 Hz, 1H), 8.49 (br d, J = 7.6 Hz, 1H), 8.06 (d, J = 8.5 Hz, 1H), 8.02 (br s, 1H), 7.90 (s, 1H), 7.70 (br d, J = 3.7 Hz, 1H), 7.26 (s, 1H), 7.16 (d, J = 4.0 Hz, 1H), 6.97 (br s, 1H), 4.12 (br s, 1H), 2.83 (d, J = 4.9 Hz, 3H), 2.79-2.64 (m, 2H), 2.01-1.73 (m. 3H), 1.53 (br s, 3H) |
| 243 | 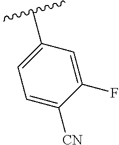 [SMILES: *c1ccc(C#N)c(F)c1] | (R)-N-(1-(4-amino-7-(4- cyano-3- fluorophenyl)pyrrolo[2,1- f][1,2,4]triazin-5- yl)piperidin-3-yl)-5- chlorothiophene-2- carboxamide, TFA | 496.29 | A: 2.04 B: 1.71 | ¹H NMR (500 MHz, DMSO-d6) δ 8.44 (br d, J = 7.9 Hz, 1H), 8.31 (br d, J = 11.9 Hz, 1H), 8.17 (br d, J = 8.2 Hz, 1H), 8.07 (br s, 1H), 7.93-7.87 (m, 2H), 7.67 (br d, J = 4.0 Hz, 1H), 7.32 (s, 1H), 7.13 (d, J = 4.0 Hz, 1H), 6.96 (br s, 1H), 4.08 (br d, J = 5.2 Hz, 1H), 3.21-3.10 (m, 1H), 3.00 (dt, J = 5.9, 2.7 Hz, 1H), 2.76-2.57 (m, 1H), 1.92-1.72 (m, 3H), 1.55-1.40 (m, 1H) |
| 244 | 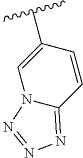 [SMILES: *c1ccc2nnnn2c1] | (R)-N-(1-(4-amino-7- (tetrazolo[1,5-a]pyridin- 6-yl)pyrrolo[2,1- f][1,2,4]triazin-5- yl)piperidin-3-yl)-5- chlorothiophene-2- carboxamide | 495.01 | A: 1.72 B: 1.53 | ¹H NMR (500 MHz, DMSO-d₆) δ 10.06 (s, 1H), 8.50- 8.41 (m, 2H), 8.22 (br d, J = 9.5 Hz, 1H), 8.09 (br s, 1H), 7.97 (s, 1H), 7.67 (br d, J = 4.0 Hz, 1H), 7.37 (S, 1H), 7.13 (d, J = 4.0 Hz, 1H), 6.97 (br s, 1H), 4.19- 4.03 (m, 1H), 3.45-3.29 (m, 1H), 3.23-3.14 (m, 1H), 3.02 (br dd, J = 7.9, 3.4 Hz, 1H), 2.73-2.64 (m, 1H), 1.96-1.76 (m, 3H), 1.57-1.38 (m, 1H) |
| 245 | 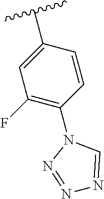 [SMILES: *c1ccc(-n2cnnn2)c(F)c1] | (R)-N-(1-(4-amino-7-(3- fluoro-4-(1H-tetrazol-1- yl)phenyl)pyrrolo[2,1- f][1,2,4]triazin-5- yl)piperidin-3-yl)-5- chlorothiophene-2- carboxamide | 539.11 | A: 1.94 B: 1.62 | ¹H NMR (500 MHz, DMSO-d₆) δ 9.94 (s, 1H), 8.49 (br d, J = 7.6 Hz, 1H), 8.43 (br d, J = 12.8 Hz, 1H), 8.23 (br d, J = 8.5 Hz, 1H), 8.04 (br s, 1H), 7.93 (s, 2H), 7.71 (br d, J = 3.7 Hz, 1H), 7.31 (s, 1H), 7.17 (d, J = 4.3 Hz, 1H), 7.06-6.86 (m, 1H), 4.19-4.03 (m, 1H), 3.29- 3.16 (m, 1H), 3.12-2.99 (m, 1H), 2.72 (br dd, J = 10.5, 2.3 Hz, 1H), 1.97-1.75 (m, 3H), 1.59-1.45 (m, 1H) |
| 246 | 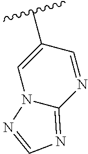 [SMILES: *c1cnc2ncnn2c1] | (R)-N-(1-(7- ([1,2,4]triazolo[1,5- alpyrimidin-6-yl)-4- aminopyrrolo[2,1- f][1,2,4]triazin-5- yl)piperidin-3-yl)-5- chlorothiophene-2- carboxamide, TFA | 495.24 | A: 1.46 B: 1.33 | ¹H NMR (500 MHz, DMSO-d₆) a 10.07 (d, J = 2.1 Hz, 1H), 9.52 (d, J = 2.4 Hz, 1H), 8.70 (s, 1H), 8.50 (brd, J = 7.6 Hz, 2H), 8.22 (br s, 1H), 8.00 (s, 1H), 7.72 (d, J = 4.0 Hz, 1H), 7.45 (s, 1H), 7.19 (d, J = 4.3 Hz, 1H), 7.06 (br s, 1H), 4.23-4.09 (m, 1H), 3.13-3.03 (m, 1H), 2.83-2.64 (m, 2H), 2.04-1.75 (m, 3H), 1,64- 1.46 (m, 1H) |
| 247 | 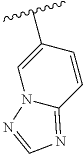 [SMILES: *c1ccc2ncnn2c1] | (R)-N-(1-(7- ([1,2,4]triazolo[1,5- alpyridin-6-yl)-4- aminopyrrolo[2,1- f][1,2,4]triazin-5- yl)piperidin-3-yl)-5- chlorothiophene-2- carboxamide | 494.29 | A: 1.60 B: 1.35 | ¹H NMR (500 MHz, DMSO-d₆) δ 9.82 (s, 1H), 8.52 (s, 1H), 8.48 (br d, J = 7.6 Hz, 1H), 8.29 (br d, J = 9.8 Hz, 1H), 8.05 (br s, 1H), 7.96 (s, 1H), 7.91 (d, J = 9.5 Hz, 1H), 7.76-7.67 (m, 1H), 7.31 (s, 1H), 7.18 (d, J = 4.0 Hz, 1H), 6.96 (br s, 1H), 4.23-4.07 (m, 1H), 3.12- 3.00 (m, 1H), 2.78-2.66 (m, 1H), 1.99-1.78 (m, 3H), 1.60-1.45 (m, 1H) |
| 248 | 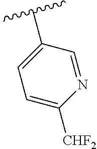 [SMILES: *c1ccc(C(F)F)nc1] | (R)-N-(1-(4-amino-7-(6- (difluoromethyl)pyridin- 3-yl)pyrrolo[2,1- f][1,2,4]triazin-5- yl)piperidin-3-yl)-5- chlorothiophene-2- carboxamide, TFA | 504.06 | A: 1.89 B: 1.60 | ¹H NMR (500 MHz, DMSO-d₆) δ 9.29 (s, 1H), 8.67 (dd, J = 8.1, 1.4 Hz, 1H), 8.49 (br d, J = 7.3 Hz, 1H), 7.98 (br s, 1H), 7.79 (d, J = 8.5 Hz, 1H), 7.70 (d, J = 4.0 Hz, 1H), 7.29 (s, 1H), 7.17 (d, J = 4.0 Hz, 1H), 7.11-6.84 (m, 1H), 4.23-4.09 (m, 1H), 3.28-3.19 (m, 1H), 3.13- 3.03 (m, 1H), 2.81-2.67 (m, 2H), 1.98-1.80 (m, 3H), 1.58-1.47 (m, 1H) |
| 249 | 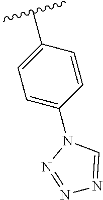 [SMILES: *c1ccc(-n2cnnn2)cc1] | (R)-N-(1-(7-(4-(1H- tetrazol-1-yl)phenyl)-4- aminopyrrolo[2,1- f][1,2,4]triazin-5- yl)piperidin-3-yl)-5- chlorothiophene-2- carboxamide, TFA | 521.33 | A: 1.85 B: 1.50 | ¹H NMR (500 MHz, DMSO-d₆) δ 10.12 (s, 1H), 8.49 (br d, J = 7.3 Hz, 1H), 8.36 (br d, J = 8.5 Hz, 2H), 7.98 (br d, J = 7.9 Hz, 2H), 7.71 (br d, J = 3.7 Hz, 1H), 7.24 (br s, 1H), 7.17 (br d, J = 4.0 Hz, 1H), 7.15 (s, 1H), 7.04 (s, 1H), 4.21-4.07 (m, 1H), 3.48-3.32 (m, 2H), 3,12- 3.02 (m, 1H), 2.81-2.65 (m, 2H), 1.99-1.76 (m, 3H), 1.58-1.45 (m, 1H) |
| 250 | 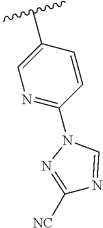 [SMILES: *c1ccc(-n2cnc(C#N)n2)nc1] | (R)-N-(1-(4-amino-7-(6- (3-cyano-1H-1,2,4- triazol-1-yl)pyridin-3- yl)pyrrolo[2,1- f][1,2,4]triazin-5- yl)piperidin-3-yl)-5- chlorothiophone-2- carboxamide, TFA | 546.08 | A: 2.01 B: 1.69 | ¹H NMR (500 MHz, DMSO-d₆) δ 9.72 (s, 1H), 9.26 (s, 1H), 8.83 (br d, J = 7.6 Hz, 1H), 8.50 (br d, J = 7.6 Hz, 1H), 8.03 (br s, 1H), 8.00 (br d, J = 8.8 Hz, 1H), 7.91 (s, 1H), 7.70 (br d, J = 3.4 Hz, 1H), 7.29 (s, 1H), 7.17 (br d, J = 4.0 Hz, 1H), 6.99 (br s, 1H), 4.13 (br dd, J = 4.3, 2.4 Hz, 1H). 3.50-3.34 (m, 2H), 2.72 (br dd, J = 10.4, 6.7 Hz, 2H), 1.97-1.77 (m, 3H), 1.59-1.46 (m, 1H) |
| 251 | 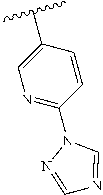 [SMILES: *c1ccc(-n2cncn2)nc1] | (R)-N-(1-(7-(6-(1H- 1,2,4-triazol-1- yl)pyridin-3-yl)-4- aminopyrrolo[2,1- f][1,2,4]triazin-5- yl)piperidin-3-yl)-5- chlorothiophene-2- carboxamide, TFA | 521.12 | A: 1.74 B: 1.45 | ¹H NMR (500 MHz, DMSO-d₆) δ 9.40 (s, 1H), 9.17 (s, 1H), 8.76-8.68 (m, 1H), 8.50 (br d, J = 7.6 Hz, 1H), 8.32 (s, 1H), 8.00 (s, 1H), 7.96 (d, J = 8.9 Hz, 1H), 7.72 (d, J = 4.0 Hz, 1H), 7.28 (s, 1H), 7.17 (d, J = 4.3 Hz, 1H), 4.22-4.11 (m, 1H), 3.26 (br d, J = 9.5 Hz, 1H), 3.09 (br d, J = 10.4 Hz, 1H), 2.80-2.68 (m, 2H), 1.97-1.83 (m, 3H), 1.59-1.46 (m, 1H) |
| 252 | 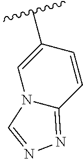 [SMILES: *c1ccc2nncn2c1] | (R)-N-(1-(7- ([1,2,4]triazolo[4,3- alpyridin-6-yl)-4- aminopyrrolo[2,1- f][1,2,4]triazin-5- yl)piperidin-3-yl)-5- chlorothiophene-2- carboxamide | 494.33 | A: 1.47 B: 1.23 | ¹H NMR (500 MHz, DMSO-d₆) δ 9.54 (s, 1H), 9.38 (s, 1H), 8.49 (br d, J = 7.3 Hz, 1H), 7.97-7.92 (m, 2H), 7.84 (d, J = 9.8 Hz, 1H), 7.71 (d, J = 4.0 Hz, 1H), 7.23 (s, 1H), 7.17 (d, J = 4.0 Hz, 1H), 4.19-4.08 (m, 1H), 3,48- 3.37 (m, 1H), 3.26-3.17 (m, 1H), 3.09-3.01 (m, 1H), 2.76-2.66 (m, 1H), 1.97-1.74 (m, 3H), 1.59-1.45 (m, 1H) |
| 253 | 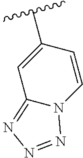 [SMILES: *c1ccn2nnnc2c1] | (R)-N-(1-(4-amino-7- (tetrazolo[ 1,5-a]pyridin- 7-yl)pyrrolo[2,1- f][1,2,4]triazin-5- yl)piperidin-3-yl)-5- chlorothiophene-2- carboxamide | 495,08 | A: 1.65 B: 1.42 | ¹H NMR (500 MHz, DMSO-d₆) δ 9.30 (br d, J = 7.0 Hz, 1H), 9.10 (s, 1H), 8.49 (br d, J = 7.0 Hz, 1H), 8.19 (br s, 1H), 8.12 (br d, J = 7.0 Hz, 1H), 8.03 (s, 1H), 7.72 (br d, J = 3.1 Hz, 1H), 7.54 (s, 1H), 7.17 (br d, J = 3.7 Hz, 1H), 7.04 (br s, 1H), 4.15 (br s, 1H), 3.06 (br s, 1H), 2.72 (br s, 1H), 1.99-1.77 (m, 3H), 1.62-1.47 (m, 1H) |
| 254 | 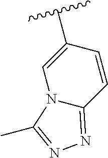 [SMILES: *c1ccc2nnc(C)n2c1] | (R)-N-(1-(4-amino-7-(3- methyl- [1,2,4]triazolo[4,3- alpyridin-6- yl)pyrrolo[2,1- f][1,2,4]triazin-5- yl)piperidin-3-yl)-5- chlorothiophene-2- carboxamide | 605.18 | A: 1.49 B: 1.29 | ¹H NMR (500 MHz, DMSO-d₆) δ 9.07 (s, 1H), 8.50 (br d, J = 7.0 Hz, 1H), 7.97 (br d, J = 9.8 Hz, 1H), 7.91 (s, 1H), 7.75 (d, J = 9.5 Hz, 1H), 7.69 (br d, J = 4.0 Hz, 1H), 7.20 (s, 1H), 7.16 (d, J = 4.3 Hz, 1H), 4,17-4.07 (m, 1H), 3.26-3.17 (m, 1H), 3.09-2.99 (m, 1H), 2.71 (s, 3H), 1.97-1.85 (m, 2H), 1.85-1.75 (m, 1H), 1.57- 1.45 (m, 1H) |

### Example 255

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methylthiophene-2-carboxamide

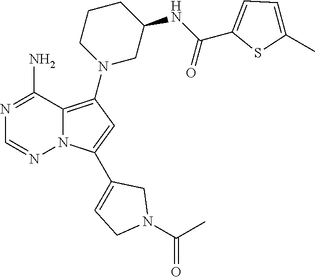 [SMILES: CC(=O)N1CC=C(c2cc(N3CCC[C@@H](NC(=O)c4ccc(C)s4)C3)c3c(N)ncnn23)C1]

To a solution of Intermediate 32 (15 mg, 0.026 mmol), 5-methylthiophene-2-carboxylic acid (4.9 mg, 0.034 mmol) and DIEA (0.023 mL, 0.13 mmol) in DMF (1.5 mL), was added HATU (13.0 mg, 0.034 mmol). The reaction mixture was stirred at it for 1 h. The reaction mixture was quenched with MeOH (0.1 mL), acidified with TFA (0.1 mL), diluted with DMF and purified by preparative HPLC to afford Example 255 (5.7 mg, 45% yield) was obtained. LC-MS Method A: RT=1.524 min, [M+H]⁺=466.1; LC-MS Method B: RT=1.294 min, [M+H]⁺=466.1; ¹H NMR (500 MHz, DMSO-d₆) δ ppm 8.22 (br d, J=7.6 Hz, 1H), 7.85 (d, J=1.2 Hz, 1H), 7.59 (br d, J=2.1 Hz, 1H), 6.82 (br s, 1H), 6.76 (br d, J=12.5 Hz, 1H), 6.69 (d, J=13.1 Hz, 1H), 4.67 (br s, 1H), 4.47 (br d, J=12.8 Hz, 2H), 4.25 (br s, 1H), 4.07 (br d, J=4.6 Hz, 1H), 3.14 (br d, J=10.1 Hz, 1H), 2.73-2.59 (m, 1H), 2.44 (s, 3H), 2.08-1.96 (m, 3H), 1.94-1.66 (m, 3H), 1.58-1.39 (m, 1H)

### Example 256

### (R)—N-(1-(7-(1-acetyl-2,5-dihydro-1H-pyrrol-3-yl)-4-aminopyrrolo[2,1-f][1,2,4]triazin-5-yl)piperidin-3-yl)-3-methoxyisothiazole-5-carboxamide

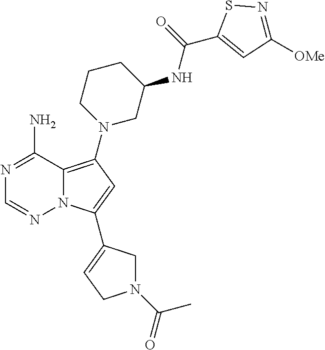 [SMILES: COc1cc(C(=O)N[C@@H]2CCCN(c3cc(C4=CCN(C(C)=O)C4)n4ncnc(N)c34)C2)sn1]

According to the synthesis of Example 255, coupling with 3-methoxyisothiazole-5-carboxylic acid afforded Example 256. LC-MS Method A: RT=1.49 min, [M+H]⁺=583.1; LC-MS Method B: RT=1.25 min. [M+H]⁺=483.3; ¹H NMR (500 MHz, DMSO-d₆) δ 8.68 (br d, J=7.6 Hz, 1H), 7.81 (d, J=1.5 Hz, 1H), 7.31 (s, 1H), 6.72 (br d, J=12.2 Hz, 1H), 6.66 (d, J=11.3 Hz, 1H), 4.63 (br s, 1H), 4.42 (br d, J=10.4 Hz, 2H), 4.22 (br s, 1H), 4.06 (dt, J=6.2, 4.8 Hz, 1H), 3.93-3.82 (m, 3H), 3.12 (br d, J=5.2 Hz, 1H), 2.94 (br d, J=3.1 Hz, 1H), 2.70-2.57 (m, 2H), 2.00 (s, 1.5H), 1.96 (s, 1.5H), 1.92-1.79 (m, 2H), 1.79-1.68 (m, 1H), 1.52-1.38 (m, 1H)

### Example 257

### (R)—N-(1-(7-(1-acetyl-2,5-dihydro-1H-pyrrol-3-yl)-4-aminopyrrolo[2,1-f][1,2,4]triazin-5-yl)piperidin-3-yl)-3-(4H-1,2,4-triazol-4-yl)benzamide, TFA

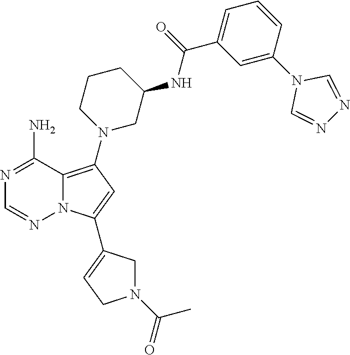 [SMILES: CC(=O)N1CC=C(c2cc(N3CCC[C@@H](NC(=O)c4cccc(-n5cnnc5)c4)C3)c3c(N)ncnn23)C1]

According to the synthesis of Example 255, coupling with 3-(4H-1,2,4-Triazol-4-yl)benzoic acid afforded Example 257. LC-MS Method A: RT=1.24 min, [M+H]⁺=512.94; LC-MS Method B: RT=1.04 min, [M+H]⁺=513.37; ¹H NMR (500 MHz, DMSO-d₆) δ 9.16 (s, 2H), 8.49 (br dd, J=7.6, 2.4 Hz, 1H), 8.09 (s, 1H), 7.97 (s, 1H), 7.87 (br dd, J=17.9, 7.8 Hz, 2H), 7.69-7.62 (m, 1H), 6.84-6.74 (m, 2H), 4.69 (br s, 1H), 4.48 (br d, J=14.6 Hz, 2H), 4.33-4.17 (m, 2H), 3.24 (br d, J=11.0 Hz, 1H), 3.11-2.98 (m, 1H), 2.81-2.64 (m, 2H), 2.04 (s, 1H), 2.00 (s, 2H), 1.98-1.91 (m, 1H), 1.92-1.79 (m, 2H), 1.62-1.48 (m, 1H)

### Example 258

### (R)—N-(1-(7-(1-acetyl-2,5-dihydro-1H-pyrrol-3-yl)-4-aminopyrrolo[2,1-f][1,2,4]triazin-5-yl)piperidin-3-yl)-3-methoxyisonicotinamide

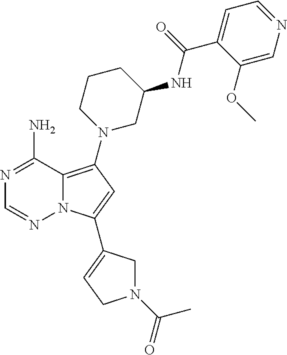 [SMILES: COc1cnccc1C(=O)N[C@@H]1CCCN(c2cc(C3=CCN(C(C)=O)C3)n3ncnc(N)c23)C1]

According to the synthesis of Example 255, coupling with 3-methoxyisonicotinic acid afforded Example 258. LC-MS Method A: RT=1.31 min, [M+H]⁺=477.1; LC-MS Method B: RT=1.02 min, [M+H]J=477.1 H NMR (500 MHz, DMSO-d₆) δ 8.51 (s, 1H), 8.37 (br s, 1H), 8.29 (d, J=4.6 Hz, 1H), 7.96 (br s, 1H), 7.86 (d, J=2.1 Hz, 1H), 7.48 (br d, J=4.6 Hz, 1H), 6.85 (br s, 1H), 6.77 (br d, J=13.7 Hz, 1H), 6.72 (d, J=12.2 Hz, 1H), 4.69 (br s, 1H), 4.53-4.41 (m, 2H), 4.26 (br s, 1H), 4.22-4.14 (m, 1H), 3.96 (s, 3H), 3.20-3.06 (m, 1H), 2.05 (s, 1.5H), 2.00 (s, 1.5H), 1.90-1.81 (m, 2H), 1.76 (br s, 1H), 1.61-1.44 (m, 1H)

### Example 259

### (R)—N-(1-(4-amino-7-(1-(2-methoxyacetyl)-2,5-dihydro-1H-pyrrol-3-yl)pyrrolo[2,1-f][1,2,4]triazin-5-yl)piperidin-3-yl)-5-chloro-3-(2-morpholinoethoxy)thiophene-2-carboxamide

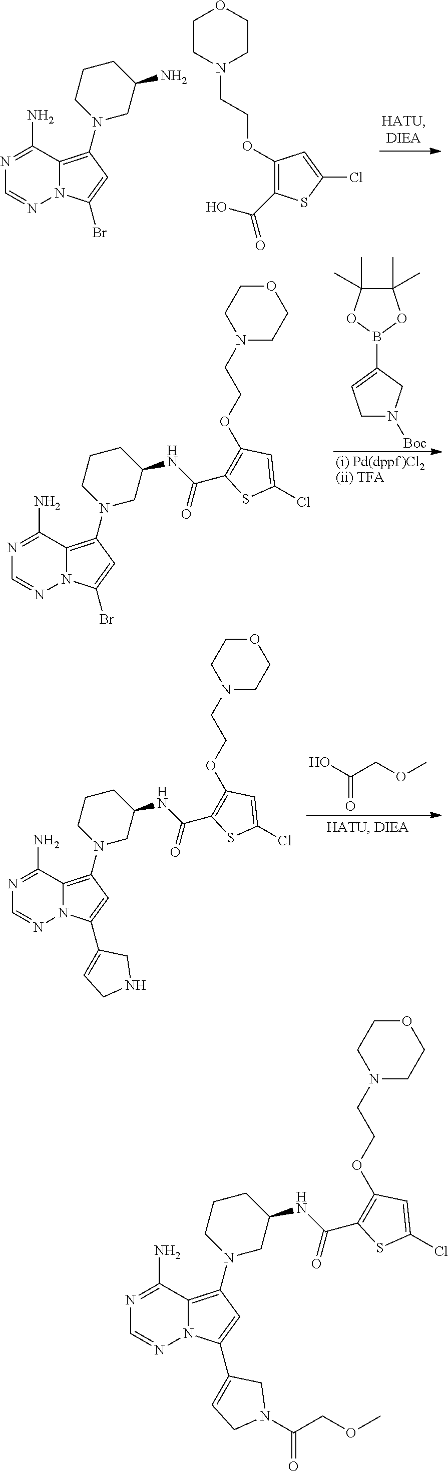

### Example 259A. (R)—N-(1-(4-amino-7-bromopyrrolo[2,1-f][1,2,4]triazin-5-yl)piperidin-3-yl)-5-chloro-3-(2-morpholinoethoxy)thiophene-2-carboxamide, 2 TFA

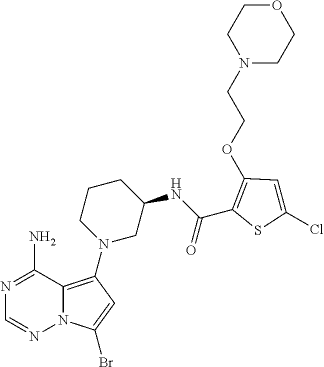 [SMILES: Nc1ncnn2c(Br)cc(N3CCC[C@@H](NC(=O)c4sc(Cl)cc4OCCN4CCOCC4)C3)c12]

To a solution of Example 171A (380 mg, 0.704 mmol), Example 142B (5-chloro-3-(2-morpholinoethoxy)thiophene-2-carboxylic acid, TFA) (300 mg, 0.739 mmol) and DIEA (0.738 mL, 4.22 mmol) in DMF (8.0 mL) at 0° C., was added HATU (281 mg, 0.739 mmol). The mixture was stirred at 0° C. for 1 h, then was quenched with MeOH (6.0 mL), acidified with TFA, and diluted with water. The mixture was purified by preparative HPLC (MeCN/H₂O/0.1% TFA) to afford Example 259A (480 mg, 84% yield) as an amber film. MS: [M+H]⁺=583.95; ¹H NMR (500 MHz, DMSO-d₆) δ ppm 8.98-8.69 (m, 1H), 7.97 (s, 1H), 7.37 (s, 1H), 7.21 (br d, J=7.9 Hz, 1H), 6.82 (s, 1H), 4.58 (t, J=4.6 Hz, 2H), 4.23-4.09 (m, 1H), 4.07-3.65 (m, 4H), 3.60 (br d, J=1.5 Hz, 3H), 3.19 (br dd, J=10.7, 3.2 Hz, 2H), 3.01 (br d, J=11.4 Hz, 1H), 2.79-2.62 (m, 2H), 1.95-1.74 (m, 3H), 1.62-1.44 (m, 1H).

### Example 259B. (R)—N-(1-(4-amino-7-(2,5-dihydro-1H-pyrrol-3-yl)pyrrolo[2,1-f][1,2,4]triazin-5-yl)piperidin-3-yl)-5-chloro-3-(2-morpholinoethoxy)thiophene-2-carboxamide, 3 TFA

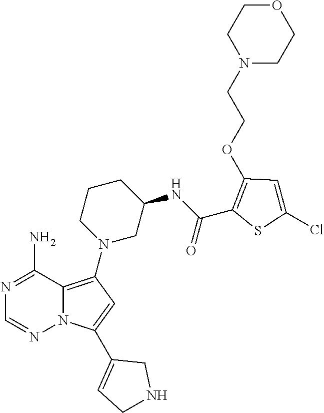 [SMILES: Nc1ncnn2c(C3=CCNC3)cc(N3CCC[C@@H](NC(=O)c4sc(Cl)cc4OCCN4CCOCC4)C3)c12]

Example 259A (480 mg, 0.590 mmol), tert-butyl 3-(4,4,5,5-tetramethyl-1,3,2-dioxaborolan-2-yl)-2,5-dihydro-1H-pyrrole-1-carboxylate (192 mg, 0.649 mmol) and PdCl₂(dppf)-CH₂Cl₂ adduct (145 mg, 0.177 mmol) were placed in a pressure vial. Then THF (10.5 mL), water (1.2 mL) and phosphoric acid, potassium salt (501 mg, 2.36 mmol) were added, and the reaction mixture was degassed (3×, vacuum/N₂). The vial was capped, and the reaction mixture was stirred at 75° C. for 14 h. The mixture was treated with Silicycle SiliaMetS DMT scavenger silica gel and was stirred for 60 min. Then the mixture was filtered, solid rinsed with THF (3×), and the mixture was concentrated. The reside was treated with TFA (8.0 mL), and stirred at it for 30 min, then concentrated. The crude material was purified by preparative HPLC (MeCN/H₂O/0.1% TFA) to afford Example 259B. (177 mg, 33% yield) as an amber glass. MS: [M+H]⁺=573.15.

### Example 259

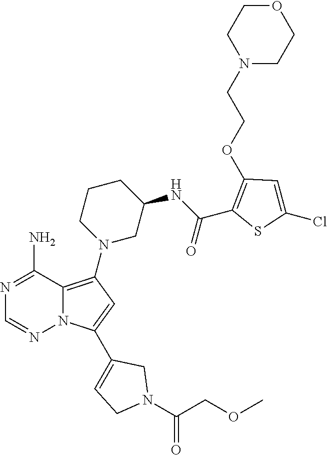 [SMILES: COCC(=O)N1CC=C(c2cc(N3CCC[C@@H](NC(=O)c4sc(Cl)cc4OCCN4CCOCC4)C3)c3c(N)ncnn23)C1]

To a mixture of Example 259B (20 mg, 0.022 mmol), 2-methoxyacetic acid (2.4 mg, 0.026 mmol) and DIEA (0.027 mL, 0.15 mmol) in DMF (1.5 mL, was added HATU (10.0 mg, 0.026 mmol). The mixture was stirred at rt for 1 h, then was quenched with MeOH and purified by preparative HPLC to afford Example 259 (7.4 mg, 51% yield). LC-MS Method A: RT=1.681 min, [M+H]⁺=645.1; LC-MS Method B: RT=1.145 min, [M+H]⁺=645.1; ¹H NMR (500 MHz, DMSO-d₆) δ ppm 7.84 (d, J=1.8 Hz, 2H), 7.56-7.43 (m, 2H), 7.28 (s, 2H), 6.78 (br d, J=1.2 Hz, 1H), 6.72 (br d, J=11.9 Hz, 2H), 6.65 (s, 1H), 4.61 (br s, 2H), 4.52 (br s, 2H), 4.40 (br s, 2H), 4.11 (s, 3H), 4.07 (s, 2H), 3.32 (d, J=6.1 Hz, 4H), 2.71-2.60 (m, 5H), 2.43-2.19 (m, 4H), 1.94-1.70 (m, 4H)

The following examples in Table 11 were prepared using the same procedure as shown in Example 259. Example 259B was coupled with the appropriate carboxylic acid. Various coupling reagents could be used other than the one described, such as BOP, PyBop, EDC/HOBt or T3P.

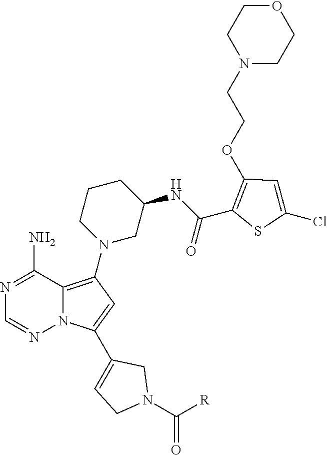

TABLE 11

| | | | | HPLC | |
|---|---|---|---|---|---|
| | | | LCMS | Method, | |
| Example | Structure (R) | Name | (M + H)⁺ | RT (min.) | ¹H NMR |
| 260 | 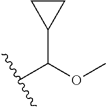 [SMILES: *C(OC)C1CC1] | N-((3R)-1-(4-amino-7-(1-(2- cyclopropyl-2-methoxyacetyl)- 2,5-dihydro-1H-pyrrol-3- yl)pyrrolo[2,1-f] [1,2,4]triazin-5- yl)piperidin-3-yl)-5-chloro-3-(2- morpholinoethoxy)thiophene-2- carboxamide | 685.2 | A: 1.818 B: 1.257 | (500 MHz, DMSO-d₆) δ ppm 7.97 (s, 1H), 7.36-7.25 (m, 3H), 6.88-6.73 (m, 2H), 4.67-4.50 (m, 5H), 4.43- 4.27 (m, 1H), 4.19-4.08 (m, 1H), 3.94-3.80 (m, 2H), 3.58 (br d, J = 3.4 Hz, 2H), 3.52-3.41 (m, 2H), 3.27- 3.23 (m, 4H), 3.23-3.13 (m, 2H), 3.02 (br d, J = 10.7 Hz, 1H), 2.83-2.66 (m, 3H), 1.97-1.74 (m, 4H), 1.58- 1.42 (m, 2H), 1.23-1.08 (m, 2H), 0.56 (br d, J = 4.3 Hz, |
| | | | | | 1H), 0.49-0.41 (m, 1H), 0.40-0.25 (m, 2H) |
| 261 | 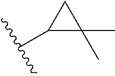 [SMILES: *C1CC1(C)C] | N-((3R)-1-(4-amino-7-(1-(2,2- dimethylcyclopropane-1- carbonyl)-2,5-dihydro-1H- pyrrol-3-yl)pyrrolo[2,1-f] [1,2,4]triazin-5-yl)piperidin-3- | 669.4 | A: 2.030 B: 1.381 | (500 MHz, DMSO-d₆) δ ppm 7.95 (d, J = 3.1 Hz, 1H), 7.34 (s, 2H), 6.82 (br d, J = 9.5 Hz, 1H), 6.76-6.68 (m, 1H), 4.71-4.61 (m, 1H), 4.56 (br s, 3H), 4.51-4.40 (m, 2H), 4.35-4.22 (m, 1H), 4.19-4.05 (m, 2H), 3.24-3.12 (m, 1H), 2.81-2.64 (m, 2H), 1.97-1.86 (m, 2H), 1.81 |
| | | yl)-5-chloro-3-(2- | | | (brs, 3H), 1.76-1.61 (m, 2H), 1.59-1.45 (m, 2H), 1.28- |
| | | morpholinocthoxy )thiophene-2- | | | 1.16 (m, 5H), 1.14-1.06 (m, 2H), 1.04-0.92 (m, 6H), |
| | | carboxamide | | | 0.76-0.64 (m, 2H) |
| 262 | 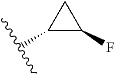 [SMILES: *[C@@H]1C[C@H]1F] | rel-N-((R)-1-(4-amino-7-(1- ((1R,2S)-2-fluorocyclopropane- 1-carbonyl)-2,5-dihydro-1H- pyrrol-3-yl)pyrrolo[2,1- f][1,2,4]triazin-5-yl)piperidin-3- | 659.1 | A: 1.857 B: 1.268 | (500 MHz, DMSO-d₆) δ ppm 7.93 (s, 1H), 7.33-7.27 (m, 1H), 7.25 (s, 1H), 6.82 (br s, 1H), 6.76 (d, J = 7.6 Hz, 1H), 4.87 (br s, 2H), 4.74 (br d, J = 6.4 Hz, 1H), 4.66 (br s, 1H), 4.56-4.45 (m, 3H), 4.28 (br s, 1H), 4.18-4.06 (m, 1H), 3.91-3.76 (m, 2H), 3.55 (br s, 2H), |
| | | yl)-5-chloro-3-(2- | | | 3.35-3.21 (m, 2H), 3.07-2.93 (m, 1H), 2.77-2.66 |
| | | morpholinoethoxy)thiophene-2- | | | (m, 2H), 2.44-2.30 (m, 1H), 1.88 (br dd, J = 11.6, 2.7 |
| | | carboxamide | | | Hz, 1H), 1.79 (br s, 2H), 1.58-1.42 (m, 2H), 1.29-1.13 |
| | | | | | (m, 1H) |
| 263 | 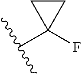 [SMILES: *C1(F)CC1] | (R)-N-(1-(4-amino-7-(1-(1- fluorocyclopropane-1-carbonyl)- 2,5-dihydro-1H-pyrrol-3- yl)pyrrolo [2,1-f][1,2,4]triazin-5- yl)piperidin-3-yl)-5-chloro-3-(2- morpholinoethoxy)thiopbene-2- | 658.9 | A: 1.967 B: 1.331 | (500 MHz, DMSO-d₆) δ ppm 7.95 (s, 1H), 7.34-7.27 (m, 1H), 7.25 (s, 1H), 6.82 (br s, 1H), 6.77 (br d, J = 19.2 Hz, 1H), 4.91 (br s, 1H), 4.70 (br s, 1H), 4.63 (br s, 1H), 4.51 (br d, J = 4.6 Hz, 2H), 4.41 (br s, 1H), 4.15-4.06 (m, 1H), 3.93-3.80 (m, 2H), 3.57 (br s, 2H), 3.38-3.23 (m, 2H), 3.21-3.11(m, 1H), 3.01 (br d, J = 11.0 Hz, 1H), |
| | | carboxamide | | | 2.80-2.66 (m, 2H), 1.95-1.86 (m, 1H), 1.83-1.74 (m, 2H), |
| | | | | | 1.60-1.43 (m, 1H), 1.38-1.26 (m, 2H), 1.22 (br d, J = 6.4 |
| | | | | | Hz, 3H) |
| 264 | 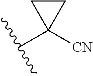 [SMILES: *C1(C#N)CC1] | (R)-N-(1-(4-amino-7-(1-(1- cyanocyclopropane-1-carbonyl)- 2,5-dihydro-1H-pyrrol-3- yl)pyrrolo[2,1-f][1,2,4]triazin-5- yl)piperidin-3-yl)-5-chloro-3-(2- | 666.1 | A: 1.844 B: 1.266 | (500 MHz, DMSO-d₆) δ ppm 7.96 (br d, J = 3.4 Hz, 1H), 7.35 (s, 1H), 7.24-7.20 (m, 1H), 6.84 (br d, J = 2.4 Hz, 1H), 6.78-6.70 (m, 1H), 4.99 (br s, 1H), 4.80 (br s, 1H), 4.66-4.52 (m, 4H), 4.38 (br d, J = 1.8 Hz, 1H), 4.20-4.08 (m, 1H), 3.23-3.15 (m, 1H), 3.06-2.96 (m, 1H), 2.81-2.67 |
| | | morpholinoethoxy)thiophene-2- | | | (m, 2H), 1.98-1.87 (m, 1H), 1.86-1.76 (m, 2H), 1.67-1.49 |
| | | carboxamide | | | (m, 6H) |
| 265 | 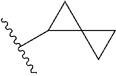 [SMILES: *C1CC12CC2] | N-((3R)-1-(4-amino-7-(1- (spiro[2.2]pentane-1-carbonyl)- 2,5-dihydro-1H-pyrrol-3- yl)pyrrolo[2, 1-f][1,2,4]triazin-5- yl)piperidin-3-yl)-5-chloro-3-(2- | 667.2 | A: 1.944 B: 1.341 | (500 MHz, DMSO-d₆) δ ppm 7.94 (s, 1H), 7.34 (s, 1H), 6.80 (br d, J = 13.7 Hz, 1H), 6.75-6.70 (m, 1H), 4.89-4.78 (m, 1H), 4.69-4.59 (m, 1H), 4.59-4.48 (m, 4H), 4.47-4.37 (m, 1H), 4.29 (br d, J = 2.1 Hz, 1H), 4.21-4.04 (m, 1H), 3.22-3.15 (m, 1H), 2.86-2.63 (m, 2H), 2.27-2.21 (m, 1H), |
| | | morpholinoethoxy)thiophene-2- | | | 2.18 (br dd, J = 7.2, 4.1 Hz, 1H), 1.99-1.86 (m, 1H), |
| | | carboxamide | | | 1.87-1.74 (m, 2H), 1.59-1.47 (m, 2H), 1.40 (br d, J = 3.4 |
| | | | | | Hz, 1H), 1.30-1.16 (m, 2H), 0.99-0.90 (m, 2H), 0.83 |
| | | | | | (br s, 3H), 0.74-0.65 (m, 1H) |
| 266 | 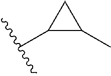 [SMILES: *C1CC1C] | N-((3R)-1-(4-amino-7-(1-(2- methylcyclopropane-1- carbonyl)-2,5-dihydro-1H- pyrrol-3-yl)pyrrolo[2,1- f][1,2,4]triazin-5-yl)piperidin-3- yl)-5-chloro-3-(2- | 655.1 | A: 1.931 B: 1.320 | (500 MHz, DMSO-d₆) δ ppm 7.96 (s, 1H), 7.34 (s, 1H), 7.24 (br d, J = 8.2 Hz, 1H), 6.82 (br s, 1H), 6.74 (d, J = 7.9 Hz, 1H), 4.84 (br s, 1H), 4.64 (br s, 1H), 4.57 (br s, 2H), 4.50 (br s, 1H), 4.28 (br d, J = 1.2 Hz, 1H), 4.19-4.08 (m, 1H), 3.88-3.69 (m, 1H), 3.22-3.12 (m, 1H), 3.06-2.95 (m, 1H), 2.81-2.64 (m, 2H), 1.94-1.75 |
| | | morpholinoethoxy)thiopbene-2- | | | (m, 4H), 1.63-1.49 (m, 2H), 1.30-1.14 (m, 2H), |
| | | carboxamide | | | 1.13-1.06 (m, 3H), 0.98 (br s, 2H), 0.67-0.59 (m, 1H) |

### Example 267

### (R)—N-(1-(4-amino-7-(2-(trifluoromethyl)pyridin-4-yl)pyrrolo[2,1-f][1,2,4]triazin-5-yl)piperidin-3-yl)-5-chloro-3-(2-morpholinoethoxy)thiophene-2-carboxamide

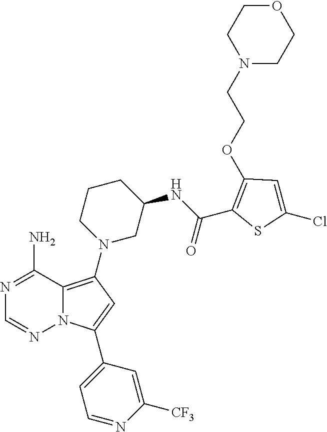 [SMILES: Nc1ncnn2c(-c3ccnc(C(F)(F)F)c3)cc(N3CCC[C@@H](NC(=O)c4sc(Cl)cc4OCCN4CCOCC4)C3)c12]

To a vial containing (2-(trifluoromethyl)pyridin-4-yl)boronic acid (5.4 mg, 0.028 mmol) and potassium phosphate (0.086 mL, 0.043 mmol), were added a solution of Example 259A (20 mg, 0.022 mmol) in THF (0.9 mL) and H₂O (0.1 mL). The mixture was degassed (evacuated and flushed with N₂, 3×). PdCl₂(dppf)-CH₂Cl₂ adduct (3.5 mg, 4.32 μmol) was added, then the mixture was degassed (3×) again. The vial was sealed and heated at 100° C. for 1 h via microwave irradiation. The mixture was treated with Pd-scavenger silica gel (~100 mg) and was stirred for 10 min. The mixture was filtered, rinsing with EtOAc, and concentrated. The mixture was concentrated and purified by preparative HPLC to afford Example 267 (7.1 mg, 33% yield). LC-MS Method B: RT=1.55 min, [M+H]⁺=651.1; 1H NMR (500 MHz, CDCl₃) δ ppm 11.91-11.71 (m, 1H), 8.86 (d, J=5.2 Hz, 1H), 8.22 (s, 1H), 8.11 (d, J=5.2 Hz, 1H), 8.08-7.95 (m, 1H), 7.84 (s, 1H), 7.06 (s, 1H), 6.73 (s, 1H), 4.74 (dt, J=11.6, 5.5 Hz, 1H), 4.68-4.60 (m, 1H), 4.38-4.28 (m, 1H), 4.02-3.98 (m, 4H), 3.56-3.50 (m, 3H), 3.35-3.24 (m, 2H), 3.21-3.16 (m, 1H), 3.14-3.07 (m, 2H), 3.06-3.01 (m, 1H), 2.03-1.96 (m, 1H), 1.95-1.89 (m, 2H), 1.87-1.81 (m, 1H), 1.05-0.71 (m, 1H).

### Example 268

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methyl-3-morpholinothiophene-2-carboxamide

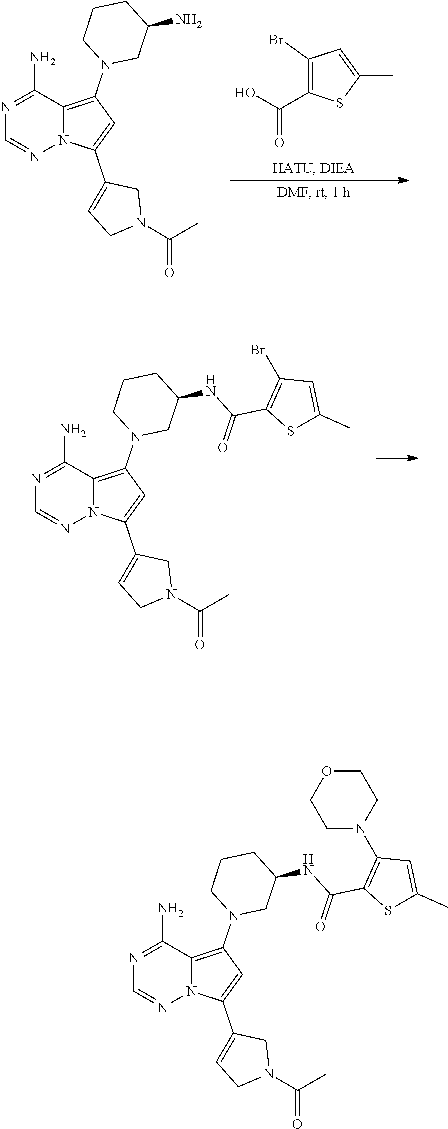

### Example 268A. (R)—N-(1-(7-(1-acetyl-2,5-dihydro-1H-pyrrol-3-yl)-4-aminopyrrolo[2,1-f][1,2,4]triazin-5-yl)piperidin-3-yl)-3-bromo-5-methylthiophene-2-carboxamide

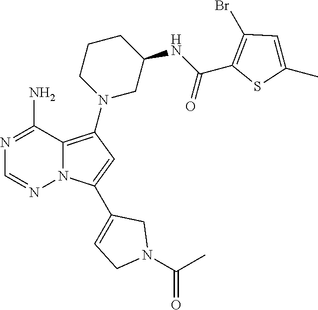 [SMILES: CC(=O)N1CC=C(c2cc(N3CCC[C@@H](NC(=O)c4sc(C)cc4Br)C3)c3c(N)ncnn23)C1]

To a mixture of Example 266B (300 mg, 0.527 mmol) and 3-bromo-5-methylthiophene-2-carboxylic acid (122 mg, 0.553 mmol) in DMF (8.0 mL, were added DIEA (0.552 mL, 3.16 mmol) and HATU (210 mg, 0.553 mmol). The mixture was stirred at rt for 1 h, then was diluted with water/(Na₂HPO₄ 1 M buffer) (80.0 mL). The resultant solid was collected, washed with water and dried to afford Example 268A (280 mg, 98% yield) as a yellow solid. MS: [M+H]⁺=544.0; ¹H NMR (500 MHz, DMSO-d₆) δ ppm 7.84 (s, 1H), 7.63 (br d, J=8.2 Hz, 1H), 7.61-7.52 (m, 1H), 7.51-7.44 (m, 1H), 7.16 (br s, 2H), 6.87 (s, 1H), 6.75 (br d, J=15.0 Hz, 1H), 6.69 (s, 1H), 4.71 (br s, 1H), 4.57-4.50 (m, 1H), 4.47 (br s, 1H), 4.29 (br d, J=1.6 Hz, 1H), 4.16 (qt, J=7.9, 3.8 Hz, 1H), 3.22 (br dd, J=1.0, 2.6 Hz, 1H), 2.89-2.80 (m, 2H), 2.46 (d, J=0.9 Hz, 3H), 2.09-2.00 (m, 2H), 1.92 (br d, J=8.0 Hz, 2H), 1.78 (br d, J=8.9 Hz, 1H), 1.68-1.58 (m, 1H)

### Example 268

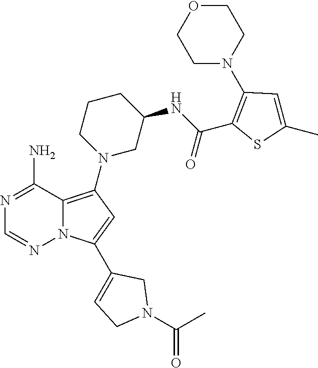 [SMILES: CC(=O)N1CC=C(c2cc(N3CCC[C@@H](NC(=O)c4sc(C)cc4N4CCOCC4)C3)c3c(N)ncnn23)C1]

Example 268A (30 mg, 0.055 mmol), Ir[dF(CF₃)ppy]₂(dtbbpy)PF₆ (1.2 mg, 1.2 μmol), morpholine (0.048 mL, 0.51 mmol) and DABCO (22 mg, 0.20 mmol) were placed in a pressure relief vial. The reaction mixture was degassed (3× vacuum/nitrogen), then a solution of NiBr₂-DME (3.4 mg, 0.011 mmol) in DMA (1.5 mL) was added. The reaction mixture was degassed again, capped and stirred under purple LED irradiation with fan cooling at rt for 9 days. SiliMetS DMT scavenger was added (30 mg; 0.64 mmol/g loading), the mixture was stirred for 15 min at rt, filtered, and the filtrate was purified by preparative HPLC to afford Example 268 (1.4 mg, 4% yield). LC-MS Method A: RT=1.515 min, [M+H]⁺=551.2. LC-MS Method B: RT=1.308 min, [M+H]⁺=551.2

### Example 269

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methyl-3-(4-methylpiperazin-1-yl)thiophene-2-carboxamide

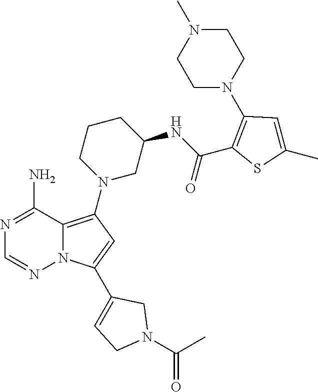 [SMILES: CC(=O)N1CC=C(c2cc(N3CCC[C@@H](NC(=O)c4sc(C)cc4N4CCN(C)CC4)C3)c3c(N)ncnn23)C1]

According to the procedure for the preparation of Example 268, coupling Example 268A (30 mg, 0.055 mmol) with 1-methylpiperazine afforded Example 269 (2.4 mg, 8% yield). LC-MS Method A: RT=1.397 min, [M+H]⁺=564.0; LC-MS Method B: RT=0.996 min, [M+H]⁺=564.0

### Example 270

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methyl-4-(4-methylpiperazin-1-yl)thiophene-2-carboxamide

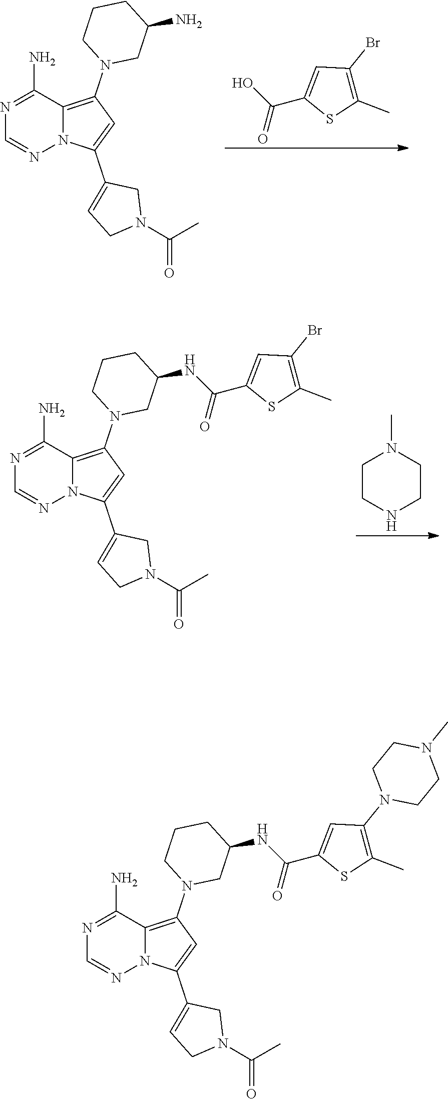

### Example 270A. (R)—N-(1-(7-(1-acetyl-2,5-dihydro-1H-pyrrol-3-yl)-4-aminopyrrolo[2,1-f][1,2,4]triazin-5-yl)piperidin-3-yl)-4-bromo-5-methylthiophene-2-carboxamide

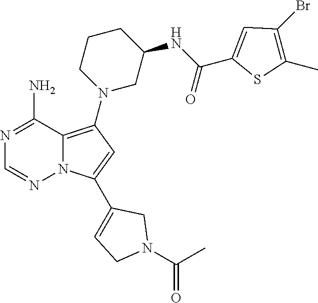 [SMILES: CC(=O)N1CC=C(c2cc(N3CCC[C@@H](NC(=O)c4cc(Br)c(C)s4)C3)c3c(N)ncnn23)C1]

To a solution of Example 255B (300 mg, 0.527 mmol) and 4-bromo-5-methylthiophene-2-carboxylic acid (122 mg, 0.553 mmol) in DMF (8.0 mL), were added DIEA (0.552 mL, 3.16 mmol) and HATU (210 mg, 0.553 mmol). The mixture was stirred at rt for 1 h. The reaction mixture was diluted with water/(Na₂HPO₄ 1 M buffer) and the resultant solid was collected, washed with water (3×10 mL) and dried to afford Example 270A (111 mg, 39% yield) as a yellow solid. MS: [M+H]⁺=543.9; ¹H NMR (500 MHz, DMSO-d₆) δ ppm 8.05 (br s, 1H), 7.83 (s, 1H), 7.73 (s, 1H), 7.66-7.53 (m, 1H), 7.51-7.43 (m, 1H), 7.16 (br s, 2H), 6.74 (br d, J=12.8 Hz, 1H), 6.67 (br s, 1H), 4.70 (br s, 1H), 4.52 (br s, 1H), 4.46 (br s, 1H), 4.29 (br s, 1H), 4.18-4.03 (m, 1H), 3.20 (br d, J=10.5 Hz, 1H), 2.81-2.68 (m, 2H), 2.39 (s, 3H), 2.10-1.99 (m, 3H), 1.98-1.86 (m, 2H), 1.78 (br d, J=10.9 Hz, 1H).

### Example 270

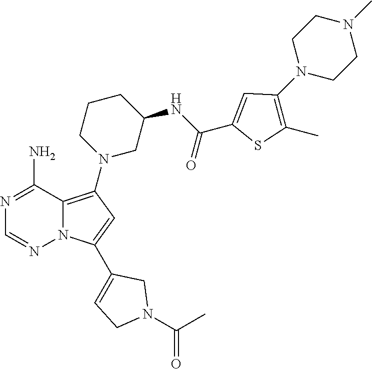 [SMILES: CC(=O)N1CC=C(c2cc(N3CCC[C@@H](NC(=O)c4cc(N5CCN(C)CC5)c(C)s4)C3)c3c(N)ncnn23)C1]

Example 270A (30 mg, 0.055 mmol), Ir[dF(CF₃)ppy]₂(dtbbpy)PF₆ (1.2 mg, 1.1 μmol), 1-methylpiperazine (0.061 mL, 0.55 mmol) and DABCO (22.3 mg, 0.198 mmol) were placed in a pressure relief vial. The reaction mixture was degassed (3× vacuum/nitrogen), then a solution of NiBr₂-DME (3.4 mg, 0.011 mmol) in DMA (1.5 mL) was added. The reaction mixture was degassed again, capped and stirred under purple LED irradiation with fan cooling at rt for 9 d. SiliMetS DMT scavenger was added, the mixture was stirred for 15 min at rt, filtered, and the filtrate was purified by preparative HPLC to afford Example 270 (1.8 mg, 5% yield).

LC-MS Method A: RT=1.300 min, [M+H]⁺=564.3; LC-MS Method B: RT=0.998 min, [M+H]⁺=564.3; ¹H NMR (500 MHz, DMSO-d₆) δ ppm 8.30-8.19 (m, 1H), 7.70 (br s, 1H), 6.77 (br d, J=8.9 Hz, 1H), 6.73 (br d, J=14.3 Hz, 1H), 4.68 (br s, 1H), 4.46 (br d, J=17.9 Hz, 2H), 4.24 (br s, 1H), 4.19-4.02 (m, 1H), 3.29-3.09 (m, 2H), 3.06-3.01 (m, 1H), 2.98 (s, 3H), 2.96-2.90 (m, 1H), 2.86 (s, 2H), 2.74-2.58 (m, 3H), 2.30 (s, 2H), 2.07-1.96 (m, 3H), 1.95-1.87 (m, 1H), 1.87-1.72 (m, 2H), 1.53-1.40 (m, 1H).

### Example 271

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methyl-4-morpholinothiophene-2-carboxamide

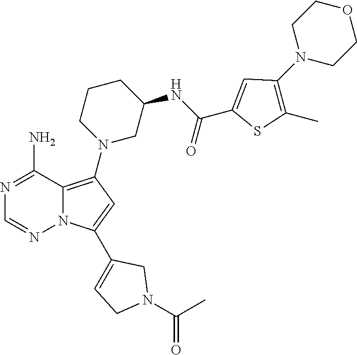 [SMILES: CC(=O)N1CC=C(c2cc(N3CCC[C@@H](NC(=O)c4cc(N5CCOCC5)c(C)s4)C3)c3c(N)ncnn23)C1]

According to the procedure for the preparation of Example 270, coupling Example 270A (30 mg, 0.055 mmol) with morpholine afforded Example 271 (0.9 mg, 3% yield). LC-MS Method A: RT=1.559 min, [M+H]⁺=551.2; LC-MS Method B: RT=1.297 min, [M+H]⁺=551.2.

### Example 272

### (R)—N-(1-(7-(1-acetyl-2,5-dihydro-1H-pyrrol-3-yl)-4-aminopyrrolo[2,1-f][1,2,4]triazin-5-yl)piperidin-3-yl)-4-morpholinothiophene-2-carboxamide

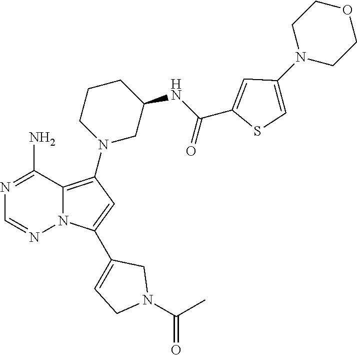 [SMILES: CC(=O)N1CC=C(c2cc(N3CCC[C@@H](NC(=O)c4cc(N5CCOCC5)cs4)C3)c3c(N)ncnn23)C1]

According to the procedure for the preparation of Example 271, amide formation between Example 255B and 4-bromothiophene-2-carboxylic acid, followed by coupling with morpholine afforded Example 272. LC-MS Method A: RT=1.352 min, [M+H]⁺=537.0; LC-MS Method B: RT=1.141 min, [M+H]⁺=537.0; ¹H NMR (500 MHz, DMSO-d₆) δ ppm 8.38 (br d, J=7.6 Hz, 1H), 8.24 (br dd, J=7.2, 2.3 Hz, 1H), 7.96 (s, 1H), 7.83-7.70 (m, 1H), 7.67 (s, 1H), 7.13 (br t, J=4.4 Hz, 1H), 6.81 (br s, 1H), 6.79-6.71 (m, 1H), 6.60 (s, 1H), 4.69 (br s, 1H), 4.50 (br s, 1H), 4.47 (br s, 1H), 4.27 (br s, 1H), 4.18-4.07 (m, 1H), 3.75-3.67 (m, 2H), 3.25-3.15 (m, 1H), 3.08-2.96 (m, 4H), 2.75-2.61 (m, 21H), 2.02 (d, J=19.8 Hz, 3H), 1.91 (br d, J=8.2 Hz, 1H), 1.84 (br s, 2H), 1.56-1.42 (m, 1H).

### Example 273

### Preparation of (R)—N-(1-(4-amino-7-(1-(methylsulfonyl)-2,5-dihydro-1H-pyrrol-3-yl)pyrrolo[2,1-f][1,2,4]triazin-5-yl)piperidin-3-yl)-5-chloro-3-(2-(4-methylpiperazin-1-yl)ethoxy)thiophene-2-carboxamide

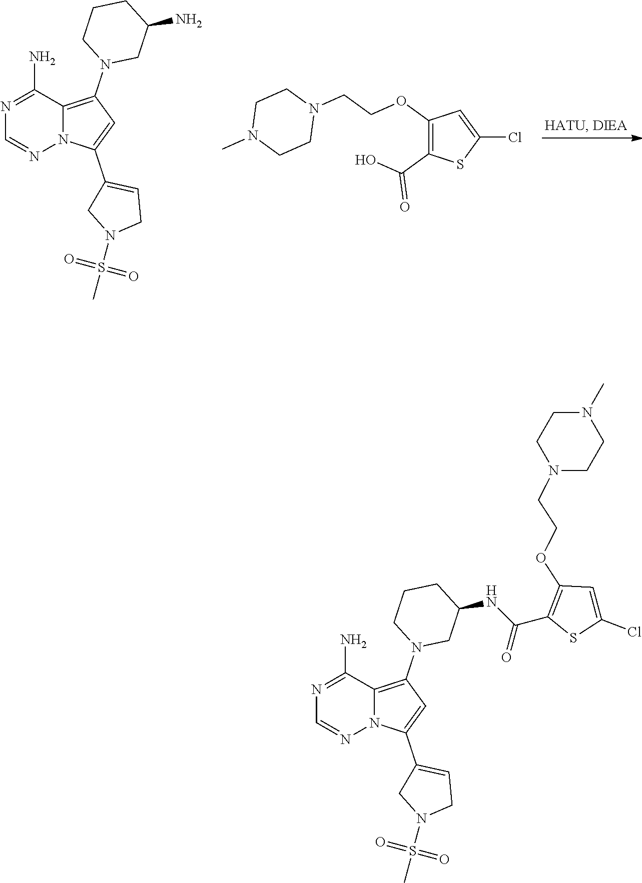 [SMILES: CS(=O)(=O)N1CC=C(c2cc(N3CCC[C@@H](N)C3)c3c(N)ncnn23)C1 ; CN1CCN(CCOc2cc(Cl)sc2C(=O)O)CC1 ; CN1CCN(CCOc2cc(Cl)sc2C(=O)N[C@@H]2CCCN(c3cc(C4=CCN(S(C)(=O)=O)C4)n4ncnc(N)c34)C2)CC1]

According to the procedure for the preparation of Example 112, reaction of Example 112G and 5-chloro-3-(2-(4-methylpiperazin-1-yl)ethoxy)thiophene-2-carboxylic acid afforded Example 273. LC-MS Method A: RT=1.640 min, [M+H]⁺=664.3; LC-MS Method B: RT=1.213 min, [M+H]⁺=664.1; ¹H NMR (500 MHz, DMSO-d₆) δ ppm 7.89 (s, 1H), 7.45-7.34 (m, 1H), 7.33 (s, 1H), 7.26 (s, 1H), 7.16 (s, 1H), 7.06 (s, 1H), 6.75 (s, 1H), 6.72 (br s, 1H), 4.53 (br s, 2H), 4.41-4.27 (m, 4H), 4.21-4.07 (m, 1H), 2.95 (s, 5H), 2.81 (br s, 3H), 2.70 (br s, 3H), 2.54 (s, 31H), 2.44-2.28 (m, 1H), 1.94-1.75 (m, 31H), 1.61-1.45 (m, 1H).

### Example 274

### Preparation of (R)—N-(1-(4-amino-7-(4-(methylsulfonyl)phenyl)pyrrolo[2,1-f][1,2,4]triazin-5-yl)piperidin-3-yl)benzenesulfonamide

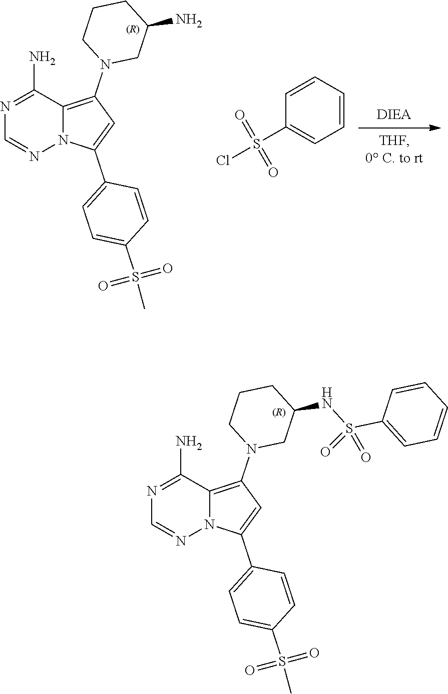

To a solution of Example 13A (10 mg, 0.024 mmol) and DIEA (0.021 mL, 0.118 mmol) in THF (1.5 mL) at 0° C., was added benzenesulfonyl chloride (3.9 μl, 0.031 mmol). The mixture was stirred at 0° C. for 15 min, and then at it for 1 h. The reaction was quenched with MeOH (0.1 mL), then was concentrated. The residue was purified by preparative HPLC to afford Example 274 (5.7 mg, 45% yield). LC-MS Method A: RT=1.58 min, [M+H]⁺=527.12; LC-MS Method B: RT=1.41 min, [M+H]⁺=527.05; ¹H NMR (500 MHz, DMSO-d₆) δ ppm 8.30 (d, J=8.5 Hz, 2H), 7.94 (d, J=8.6 Hz, 2H), 7.87 (s, 1H), 7.85-7.80 (m, 2H), 7.61-7.48 (m, 3H), 7.07 (br s, 1H), 3.47-3.33 (m, 1H), 2.97-2.82 (m, 1H), 1.84-1.69 (m, 1H), 1.68-1.50 (m, 2H), 1.35-1.20 (m, 1H).

### Example 275

### N-((3R)-1-(4-amino-7-(1-(tetrahydrofuran-3-yl)-1H-pyrazol-4-yl)pyrrolo[2,1-f][1,2,4]triazin-5-yl)piperidin-3-yl)-5-chloro-3-(2-(dimethylamino)ethoxy)thiophene-2-carboxamide

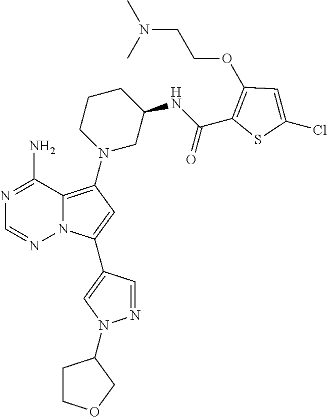 [SMILES: CN(C)CCOc1cc(Cl)sc1C(=O)N[C@@H]1CCCN(c2cc(-c3cnn(C4CCOC4)c3)n3ncnc(N)c23)C1]

According to the procedure for the preparation of Example 186, Example 171B (20 mg, 0.037 mmol) was coupled with 1-(oxolan-3-yl)-1H-pyrazole-4-boronic acid pinacol ester to afford Example 275. LC-MS Method A: RT=1.75 min, [M+H]⁺=600.2; LC-MS Method B: RT=1.14 min, [M+H]⁺=600.1.

### Example 276

### Preparation of (R)—N-(1-(4-amino-7-(4-formylphenyl)pyrrolo[2,1-f][1,2,4]triazin-5-yl)piperidin-3-yl)-5-chloro-3-(2-(dimethylamino)ethoxy)thiophene-2-carboxamide

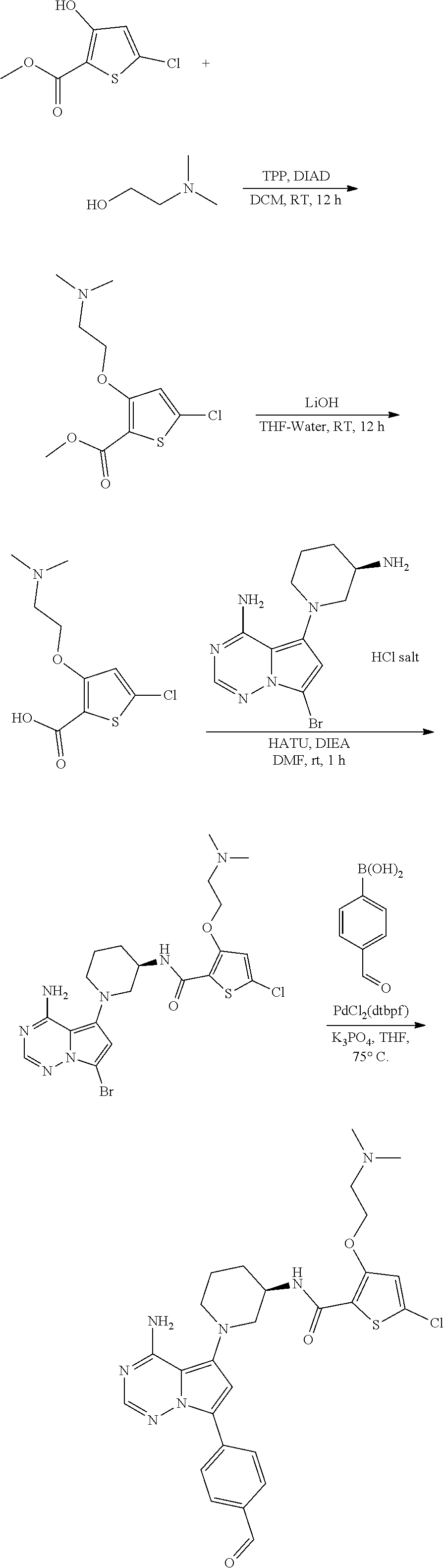

### Example 276A. Methyl 5-chloro-3-(2-(dimethylamino)ethoxy)thiophene-2-carboxylate

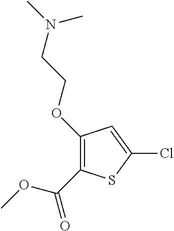 [SMILES: COC(=O)c1sc(Cl)cc1OCCN(C)C]

DIAD (1.67 mL, 8.57 mmol) was added dropwise to a solution of methyl 5-chloro-3-hydroxythiophene-2-carboxylate (1.5 g, 7.79 mmol), 2-(dimethylamino)ethan-1-ol (0.694 g, 7.79 mmol) and triphenylphosphine (2.451 g, 9.35 mmol) in DCM (30 mL) kept at 0° C. The mixture was stirred at rt for 12 h and evaporated under reduced pressure. The residue was purified by flash chromatography (5% DCM/MeOH) to afford Example 276A (1.00 g, 49% yield) as a yellow solid. MS: [M+H]⁺=264.2; ¹H NMR (500 MHz, DMSO-d₆) δ ppm 7.42 (s, 1H), 4.53-4.47 (m, 2H), 3.76-3.70 (m, 3H), 3.55-3.49 (m, 2H), 2.92 (s, 6H).

### Example 276B. 5-Chloro-3-(2-(dimethylamino)ethoxy)thiophene-2-carboxylic acid

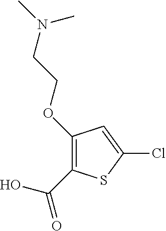

To a solution of methyl 5-chloro-3-(2-(dimethylamino)ethoxy)thiophene-2-carboxylate (460 mg, 1.744 mmol) in THF (5 mL), was added LiOH (3.5 mL of 1M aq. solution, 3.49 mmol). Reaction mixture was stirred at it for 8 h and concentrated under reduced pressure to afford Example 276B (420 mg, 96% yield) as an off-white solid. MS: [M+H]⁺=250.2.

### Example 276C. (R)—N-(1-(4-amino-7-bromopyrrolo[2,1-f][1,2,4]triazin-5-yl)piperidin-3-yl)-5-chloro-3-(2-(dimethylamino)ethoxy)thiophene-2-carboxamide

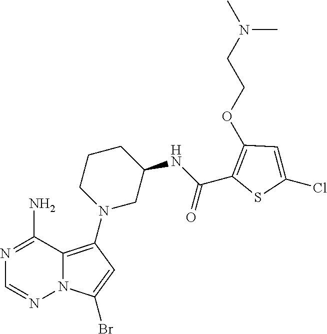 [SMILES: CN(C)CCOc1cc(Cl)sc1C(=O)N[C@@H]1CCCN(c2cc(Br)n3ncnc(N)c23)C1]

DIEA (2.021 mL, 11.57 mmol) was added to a solution of (R)-5-(3-aminopiperidin-1-yl)-7-bromopyrrolo[2,1-f][1,2,4]triazin-4-amine (1.2 g, 3.86 mmol), 5-chloro-3-(2-(dimethylamino)ethoxy)thiophene-2-carboxylic acid (0.963 g, 3.86 mmol) and HATU (1.466 g, 3.86 mmol) in DMF (15 mL). The mixture was stirred at rt for 2 h, diluted with ethyl acetate (100 mL) and washed with water (150 mL). The organic layer was separated, dried over sodium sulphate and concentrated under reduced pressure. The residue was purified by flash chromatography (7% DCM/MeOH) to afford Example 276C (1.2 g, 2.210 mmol, 57.3% yield) as a yellow solid. MS: [M+H]⁺=544.0.

### Example 276

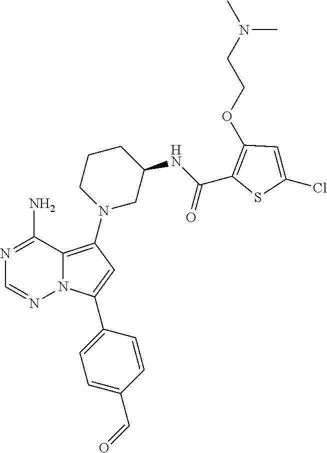 [SMILES: CN(C)CCOc1cc(Cl)sc1C(=O)N[C@@H]1CCCN(c2cc(-c3ccc(C=O)cc3)n3ncnc(N)c23)C1]

[1,1′-Bis(di-tert-butylphosphino)ferrocene]dichloropalladium(I) (6.00 mg, 9.21 μmol) was added to a mixture of (R)—N-(1-(4-amino-7-bromopyrrolo[2,1-f][1,2,4]triazin-5-yl)piperidin-3-yl)-5-chloro-3-(2-(dimethylamino)ethoxy)thiophene-2-carboxamide (50 mg, 0.092 mmol), 4-formylphenylboronic acid (13.81 mg, 0.092 mmol) and Na₂CO₃ (0.138 mL, 0.276 mmol) in THF (3 mL). The reaction mixture was heated to 75° C. for 6 h. Cooled to rt, filtered and the filtrate was concentrated and purified by preparative HPLC to afford Example 276 (2.5 mg, 4.6% yield). LCMS Method E: RT=2.00 min, [M+H]⁺=568.2; LC-MS Method F: RT=1.21 min, [M+H]⁺=568.2; ¹H-NMR (400 MHz, DMSO-d₆) δ ppm 10.06 (s, 1H), 8.41-8.30 (m, 2H), 8.21-8.01 (m, 1H), 7.96 (d, J=8.3 Hz, 2H), 7.91 (s, 1H), 7.74 d, J=6.3 Hz, 1H), (7.35 (s, 1H), 7.25 (s, 1H), 6.91 (bs, 1H), 4.33-4.26 (m, 2H), 4.23-4.12 (m, 1H), 3.93-3.89 (m, 1H), 3.22-3.11 (m, 1H), 3.11-2.70 (m, 4H), 2.18 (s, 6H), 2.03-1.69 (m, 3H), 1.62-1.40 (m, 1H).

The following examples in Table 12 were prepared using the same procedure as shown in Example 276. Example 276C was coupled with the appropriate boronic acid or boronate ester, using potassium phosphate tribasic monohydrate (2M aq. soln.) and [1,1′-bis(di-tert-butylphosphino)ferrocene]dichloropalladium(II)] in THF at 75° C. for 6 h.

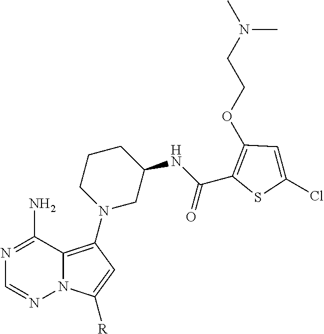

TABLE 12

| | | | | HPLC | |
|---|---|---|---|---|---|
| | | | LCMS | Method, | |
| Example | Structure (R) | Name | (M + H)⁺ | RT (min.) | ¹H NMR |
| 277 | 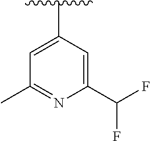 [SMILES: *c1cc(C)nc(C(F)F)c1] | (R)-N-(1-(4-amino-7-(2- (difluoromethyl)-6- methylpyridin-4-yl)pyrrolo[2,1- f][1,2,4]triazin-5-yl)piperidin-3- yl)-5-chloro-3-(2- (dimethylamino)ethoxy) thiophene-2-carboxamide | 605.20 | E: 2.07 F: 1.22 | (400 MHZ, DMSO-d₆) δ ppm 8.31 (s, 1H), 8.14 (s, 2H), 7.97 (s, 1H), 7.70 (br s, 1H), 7.42 (s, 1H), 7.35 (s, 1H), 6.91 (t, J = 54.5 Hz, 2H), 4.30 (br t, J = 5.1 Hz, 2H), 4.17 (br s, 1H), 3.17 (br s, 1H), 3.02 (br s, 1H), 2.81 (br s, 2H), 2.64-2.54 (m, 5H), 2.20 (br s, 6H), 1.87 (br s, 3H), 1.50 (br s, 1H) |
| 278 | 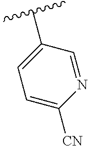 [SMILES: *c1ccc(C#N)nc1] | (R)-N-(1-(4-amino-7-(6- cyanopyridin-3-yl)pyrrolo[2,1- f][1,2,4]triazin-5-yl)piperidin-3- yl)-5-chloro-3-(2- (dimethy lamino)ethoxy) thiophene-2-carboxamide | 566.20 | G: 1.58 | (400 MHZ, DMSO-d₆) δ ppm 9.51-9.33 (m, 1H), 8.87-8.73 (m, 1H), 8.15-8.05 (m, 1H), 8.10-8.08 (m, 1H), 7.95 (s, 1H), 7.83-7.66 (m, 1H), 7.43-7.40 (m, 1H), 7.37-7.33 (m, 1H), 6.93 (br s, 1H), 4.39-4.25 (m, 2H), 4.25-4.10 (m, 2H), 3.21-2.95 (m, 5H), 2.18 (s, 6H), 2.00-1.74 (m, 3H), 1.57-1.41 (m, 1H) |
| 279 | 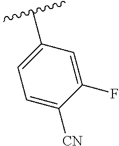 [SMILES: *c1ccc(C#N)c(F)c1] | (R)-N-(1-(4-amino-7-(4-cyano- 3-fluorophenyl)pyrrolo[2,1- f][1,2,4]triazin-5-yl)piperidin-3- yl)-5-chloro-3-(2- (dimethy lamino)ethoxy) thiophene-2-carboxamide | 583.10 | E: 2.20 F: 1.31 | (400 MHZ, DMSO-d₆) δ ppm 9.88-9.69 (m, 1H), 8.39-8.32 (m, 1H), 8.21 (br d, J = 1.5 Hz, 1H), 7.98 (s, 2H), 7.37 (d, J = 8.3 Hz, 2H), 7.23 (s, 1H), 6.93 (br s, 1H), 4.63-4.47 (m, 2H), 4.23-4.05 (m, 1H), 3.29-3.19 (m, 2H), 3.10-2.98 (m, 2H), 2.86 (br s, 8H), 2.03-1.77 (m, 3H), 1.69-1.49 (m, 1H) |
| 280 | 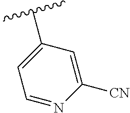 [SMILES: *c1ccnc(C#N)c1] | (R)-N-(1-(4-amino-7-(2- cyanopyridin-4-yl)pyrrolo[2,1- f][1,2,4]triazin-5-yl)piperidin-3- yl)-5-chloro-3-(2- (dimethylamino)ethoxy) thiophene-2-carboxamide | 566.10 | E: 1.94 F: 1.14 | (400 MHZ, DMSO-d₆) δ ppm 8.78-8.68 (m, 2H), 8.55-8.47 (m, 1H), 8.26 (br s, 1H), 8.01 (s, 1H), 7.73 (br d, J = 6.8 Hz, 1H), 7.53 (s, 1H), 7.35 (s, 1H), 6.96 (br s, 1H), 4.29 (t, J = 5.3 Hz, 2H), 4.23-4.07 (m, 1H), 3.23-3.12 (m, 1H), 3.01 (br s, 1H), 2.81 (br s, 2H), 2.60 (br s, 2H), 2.19 (br s, 6H), 1.88 (br s, 3H), 1.49 (br s, 1H) |
| 281 | 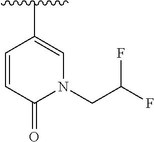 [SMILES: *c1ccc(=O)n(CC(F)F)c1] | (R)-N-(1-(4-amino-7-(1-(2,2- difluoroethyl)-6-oxo-1,6- dihydropyridin-3-yl)pyrrolo[2,1- f][1,2,4]triazin-5-yl)piperidin-3- yl)-5-chloro-3-(2- (dimethylamino)ethoxy) thiophene-2-carboxamide | 621.20 | E: 1.63 F: 1.02 | (400 MHZ, DMSO-d₆) δ ppm 8.50 (d, J = 2.3 Hz, 1H), 8.11 (dd, J = 2.5, 9.5 Hz, 1H), 7.82 (s, 1H), 7.69-7.72 (m, 2H), 7.36 (s, 1H), 6.92 (s, 1H), 6.80 (br s, 1H), 6.57 (d, J = 9.5 Hz, 1H), 6.51-6.21 (m, 1H), 4.46 (dt, J = 3.9, 14.7 Hz, 2H), 4.30 (br t, J = 5.1 Hz, 2H), 4.17 (br s, 1H), 3.17 (br s, 1H), 3.05-2.97 (m, 1H), 2.87-2.57 (m, 4H), 2.20 (br s, 6H), 1.88 (br s, 3H), 1.49 (br s, 1H) |
| 283 | 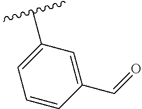 [SMILES: *c1cccc(C=O)c1] | (R)-N-(1-(4-amino-7-(3- formylphenyl)pyrrolo[2,1- f][1,2,4]triazin-5-yl)piperidin-3- yl)-5-chloro-3-(2- (dimethy lamino)ethoxy) thiophene-2-carboxamide | 568.20 | E: 1.94 F: 1.13 | (400 MHZ, DMSO-d₆) δ ppm 10.08 (s, 1H), 9.85 (br s, 1H), 8.60 (s, 1H), 8.43-8.37 (m, 1H), 7.95 (s, 1H), 7.88 (d, J = 7.8 Hz, 1H), 7.71 (t, J = 7.6 Hz, 1H), 7.38 (s, 1H), 7.25 (br d, J = 6.0 Hz, 1H), 7.16 (s, 1H), 6.95 (br s, 1H), 4.57 (br t, J = 4.5 Hz, 2H), 4.17 (br s, 1H), 3.57-3.54 (m, 2H), 3.28 (s, 1H), 3.15-3.06 (m, 1H), 2.86 (br s, 8H), 2.01-1.78 (m, |
| | | | | | 3H), 1.61 (br s, 1H) |

### Example 284

### Preparation of methyl (R)-3-(4-amino-5-(3-(5-chloro-3-(2-(4,4-difluoropiperidin-1-yl)ethoxy)thiophene-2-carboxamido)piperidin-1-yl)pyrrolo[2,1-f][1,2,4]triazin-7-yl)-2,5-dihydro-1H-pyrrole-1-carboxylate

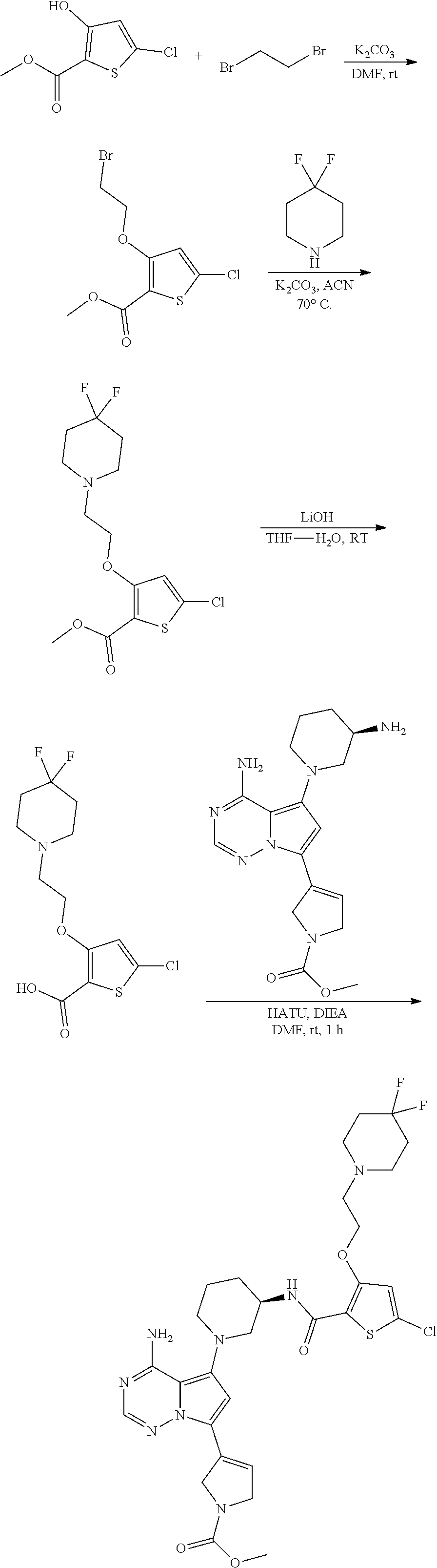

### Example 284A. Methyl 3-(2-bromoethoxy)-5-chlorothiophene-2-carboxylate

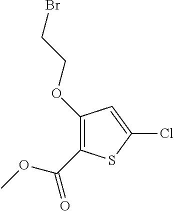

1,2-Dibromoethane (6.74 mL, 78 mmol) was added dropwise to the suspension of methyl 5-chloro-3-hydroxythiophene-2-carboxylate (3 g, 15.58 mmol) and K₂CO₃ (3.01 g, 21.81 mmol) in DMF (50 mL). The mixture was stirred at rt for 4 h, diluted with ethyl acetate (50 mL) and washed with water (100 mL). The organic layer was separated, dried over sodium sulphate and concentrated under reduced pressure. The residue was purified by flash chromatography (25% Pet. Ether/EtOAc) to afford Example 284A (2.30 g, 49.3% yield) as an off-white solid. MS: [M+H]⁺=301.0; ¹H NMR (400 MHz, CDCl₃) δ ppm 6.76 (s, 1H), 4.43-4.40 (t, 2H), 3.85 (s, 3H), 3.67-3.64 (t, 2H).

### Example 284B. methyl 5-chloro-3-(2-(4,4-difluoropiperidin-1-yl)ethoxy)thiophene-2-carboxylate

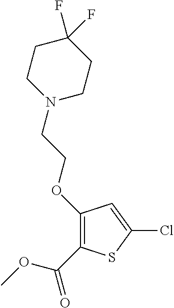 [SMILES: COC(=O)c1sc(Cl)cc1OCCN1CCC(F)(F)CC1]

K₂CO₃ (346 mg, 2.50 mmol) was added to a solution of methyl 3-(2-bromoethoxy)-5-chlorothiophene-2-carboxylate (150 mg, 0.501 mmol) and 4,4-difluoropiperidine (182 mg, 1.50 mmol) in acetonitrile (2 mL). It was heated at 70° C. for 6 h. The reaction mixture was partitioned between water (10 mL) and ethyl acetate (50 mL). The organic layer was separated, dried over sodium sulphate and concentrated under reduced pressure to afford Example 284B (160 mg, 94% yield) as a yellow oil. MS: [M+H]⁺=340.2.

### Example 284C. 5-chloro-3-(2-(4,4-difluoropiperidin-1-yl)ethoxy)thiophene-2-carboxylic acid

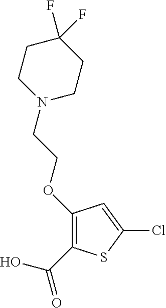 [SMILES: O=C(O)c1sc(Cl)cc1OCCN1CCC(F)(F)CC1]

LiOH (15.9 mg, 0.662 mmol) was added to a solution of methyl 5-chloro-3-(2-(4,4-difluoropiperidin-1-yl)ethoxy)thiophene-2-carboxylate (150 mg, 0.441 mmol) in a mixture of THF (3 mL)-Water (1 mL) and stirred at rt for 12 h. The reaction mixture was acidified with aq. 1N HCl till pH 5 and evaporated to dryness to afford Example 284C (130 mg, 90% yield) as an off-white solid. MS: [M+H]⁺=326.2.

### Example 284

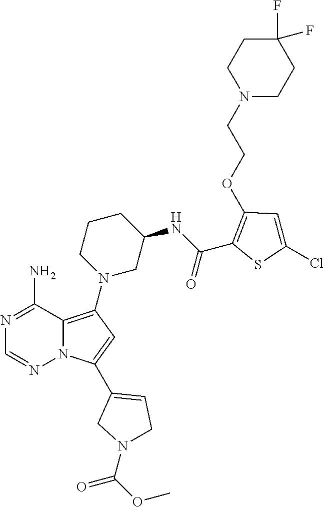 [SMILES: COC(=O)N1CC=C(c2cc(N3CCC[C@@H](NC(=O)c4sc(Cl)cc4OCCN4CCC(F)(F)CC4)C3)c3c(N)ncnn23)C1]

DIEA (0.073 mL, 0.420 mmol) was added to a solution of methyl (R)-3-(4-amino-5-(3-aminopiperidin-1-yl)pyrrolo[2,1-f][1,2,4]triazin-7-yl)-2,5-dihydro-1H-pyrrole-1-carboxylate (50 mg, 0.140 mmol), 5-chloro-3-(2-(4,4-difluoropiperidin-1-yl)ethoxy)thiophene-2-carboxylic acid (45.6 mg, 0.140 mmol) and HATU (53.2 mg, 0.140 mmol) in DMF (2 mL). The mixture was stirred at rt for 2 h, concentrated and the residue was purified by preparative HPLC to afford Example 284 (7.1 mg, 7.4% yield). LC-MS Method E: RT=1.94 min, [M+H]⁺=665.2; LC-MS Method F: RT=1.12 min, [M+H]⁺=665.2; ¹H-NMR (400 MHz, DMSO-d₆) δ ppm 8.29 (s, 1H), 7.93 (s, 1H), 7.37 (s, 1H), 7.31-7.21 (m, 1H), 7.14-6.86 (m, 1H), 6.78-6.72 (m, 2H), 4.53 (br d, J=5.4 Hz, 4H), 4.38-4.25 (m, 2H), 4.16 (br dd, J=1.5, 2.7 Hz, 1H), 3.66 (d, J=3.7 Hz, 3H), 3.29-3.15 (m, 4H), 3.04-2.88 (m, 2H), 2.80-2.68 (m, 2H), 2.28-2.10 (m, 4H), 1.96-1.72 (m, 5H), 1.61-1.44 (m, 1H).

The following examples in Table 13 were prepared using the same procedure as shown in Example 284. Methyl (R)-3-(4-amino-5-(3-aminopiperidin-1-yl)pyrrolo[2,1-f][1,2,4]triazin-7-yl)-2,5-dihydro-1H-pyrrole-1-carboxylate was coupled with the appropriate carboxylic acid using HATU and DIEA.

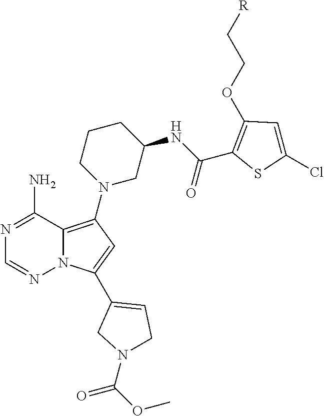

TABLE 13

| | | | | HPLC | |
|---|---|---|---|---|---|
| | | | LCMS | Method, | |
| Example | Structure (R) | Name | (M + H)⁺ | RT (min.) | ¹H NMR |
| 285 | 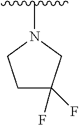 [SMILES: *N1CCC(F)(F)C1] | Methyl (R)-3-(4-amino-5- (3-(5-chloro-3-(2- (3,3-difluoropyrrolidin-1- yl)ethoxy)thiophene-2- carboxamido)piperidin-1-yl) pyrrolo[2,1-f][1,2,4]triazin-7- yl)-2,5-dihydro-1H-pyrrole- 1-carboxylate | 651.20 | E: 1.92 F: 1.22 | (400 MHZ, DMSO-d₆) δ ppm 7.86 (s, 2H), 7.57- 7.40 (m, 1H), 7.34 (s, 1H), 6.87-6.59 (m, 3H), 4.60- 4.46 (m, 2H), 4.39-4.22 (m, 4H), 4.19-4.06 (m, 1H), 3.66 (d, J = 3.9 Hz, 3H), 3.14-3.06 (m, 1H), 3.00-2.66 (m, 9H), 2.26-2.09 (m, 2H), 1.99-1.64 (m, 3H), 1.58-1.37 (m, 1H) |
| 286 | 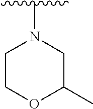 [SMILES: *N1CCOC(C)C1] | Methyl 3-(4-amino-5-((3R)- 3-(5-chloro-3-(2-(2- methylmorpholino)ethoxy) thiophene-2-carboxamido) piperidin-1-yl)pyrrolo[2,1-f] [1,2,4]triazin-7-yl)-2,5-dihydro- 1H-pyrrole-1-carboxylate | 645.20 | E: 1.76 F: 1.06 | (400 MHZ, DMSO-d₆) δ ppm 7.86 (s, 1H), 7.53- 7.39 (m, 1H), 7.35 (s, 1H), 6.76-6.67 (m, 2H), 4.53 (td, J = 4.2, 9.3 Hz, 2H), 4.38-4.22 (m, 4H), 4.20- 4.06 (m, 1H), 3.66 (d, J = 3.9 Hz, 4H), 3.13-2.75 (m, 6H), 2.71-2.61 (m, 4H), 1.99-1.74 (m, 4H), 1.68-1.48 (m, 2H), 1.00-0.91 (m, 3H) |
| 287 | 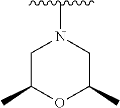 | Methyl 3-(4-amino-5-((R)-3-(5- chloro-3-(2-((2S,6R)-2,6- dimethylmorpholino)ethoxy) thiophene-2-carboxamido) piperidin-1-yl)pyrrolo[2,1-f] [1,2,4]triazin-7-yl)-2,5-dihydro- 1H-pyrrole-1-carboxylate | 659.20 | E: 1.81 F: 1.08 | (400 MHZ, DMSO-d₆) δ ppm 7.86 (s, 1H), 7.50 (br s, 1H), 7.35 (s, 1H), 6.78-6.66 (m, 2H), 4.51 (br s, 2H), 4.35-4.23 (m, 4H), 4.21-4.11 (m, 1H), 3.66 (d, J = 3.9Hz, 3H), 3.49-3.39 (m, 2H), 3.14-3.05 (m, 2H), 2.96-2.72 (m, 6H), 2.64 (br s, 1H), 1.89-1.69 (m, 3H), 1.63-1.43 (m, 2H), 0.96 (dd, J = 6.2, 12.3 Hz, 6H) |
| 288 | 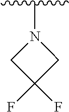 [SMILES: *N1CC(F)(F)C1] | Methyl (R)-3-(4-amino-5-(3- (5-chloro-3-(2-(3,3- difluoroazetidin-1- yl)ethoxy)thiophene-2- carboxamido)piperidin-1-yl) pyrrolo[2,1-f][1,2,4]triazin-7- yl)-2,5-dihydro-1H-pyrrole- | 637.20 | E: 1.86 F: 1.21 | (400 MHZ, DMSO-d₆) δ ppm 8.67 (br s, 1H), 7.97 (s, 1H), 7.48 (br d, J = 7.3 Hz, 1H), 7.34 (s, 1H), 7.28 (br s, 1H), 6.87-6.69 (m, 2H), 4.61-4.46 (m, 3H), 4.38-3.76 (m, 8H), 3.66 (d, J = 3.9 Hz, 3H), 3.27-2.97 (m, 4H), 2.83-2.65 (m, 2H), 2.01-1.72 (m, 3H), 1.62-1.43 (m, 1H) |
| | | 1-carboxylate | | | |
| 289 | 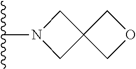 [SMILES: *N1CC2(COC2)C1] | Methyl (R)-3-(5-(3-(3-(2-(2- oxa-6-azaspiro[3.3]heptan-6-yl) ethoxy)-5-chlorothiophene-2- carboxamido)piperidin-1-yl)-4- aminopyrrolo[2,1-f][1,2,4]triazin- | 643.2 | K: 4.261 | (400 MHZ, DMSO-d₆) δ ppm 7.99 (br s, 1H), 7.90- 7.81 (m, 1H), 7.74-7.50 (m, 1H), 7.31 (s, 1H), 7.28 (br s, 1H), 6.98-6.59 (m, 2H), 4.69-4.41 (m, 6H), 4.36-4.24 (m, 2H), 4.21-4.03 (m, 3H), 3.66 (d, J = 4.5 Hz, 3H), 3.27-3.19 (m, 2H), 3.16-3.03 (m, |
| | | 7-yl)-2,5-dihydro-1H-pyrrole-1- | | | 1H), 2.99-2.87 (m, 2H), 2.74-2.59 (m, 4H), 1.93- |
| | | carboxylate | | | 1.75 (m, 3H), 1.66-1.41 (m, 1H) |
| 290 | 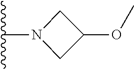 [SMILES: *N1CC(OC)C1] | Methyl (R)-3-(4-amino-5-(3-(5- chloro-3-(2-(3-methoxyazetidin- 1-yl)ethoxy)thiophene-2- carboxamido)piperidin-1-yl)pyrrolo [2,1-f][1,2,4]triazin-7-y])-2,5- | 631.3 | K: 8.586 | (400 MHZ, DMSO-d₆) δ ppm 8.13 (br s, 1H), 7.96- 7.80 (m, 1H), 7.34 (s, 1H), 7.21 (br s, 1H), 6.91- 6.55 (m, 3H), 4.62-4.48 (m, 3H), 4.41 (d, J = 4.4 Hz, 3H), 4.34-4.24 (m, 2H), 4.19-4.09 (m, 2H), 4.04-3.92 (m, 2H), 3.65 (d. J = 4.3 Hz, 6H), 3.04- |
| | | dihydro-1H-pyrrole-1-carboxylate | | | 2.91 (m, 2H), 2.79-2.63 (m, 3H), 1.96-1.77 (m, |
| | | | | | 3H), 1.61-1.45 (m, 1H) |
| 291 | 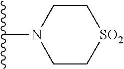 [SMILES: *N1CCS(=O)(=O)CC1] | Methyl (R)-3-(4-amino-5-(3-(5- chloro-3-(2-(1,1- dioxidothiomorpholino)ethoxy) thiophene-2-carboxamido) piperidin-1-yl)pyrrolo[2,1-f] | 679.1 | E: 1.572 F: 1.244 | (400 MHZ, DMSO-d₆) δ ppm 7.86 (s, 1H), 7.36 (s, 2H), 6.82-6.61 (m, 2H), 4.55 (br s, 2H), 4.39-4.24 (m, 4H), 4.21-4.07 (m, 1H), 3.66 (d, J = 4.2 Hz, 3H), 3.19-2.70 (m, 14H), 1.96-1.74 (m, 3H), 1.61- 1.42 (m, 1H) |
| | | [1,2,4]triazin-7-yl)-2,5-dihydro- | | | |
| | | 1H-pyrrole-1-carboxylate | | | |
| 292 | 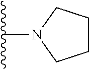 [SMILES: *N1CCCC1] | Methyl (R)-3-(4-amino-5-(3-(5- chloro-3-(2-(pyrrolidin-1-yl) ethoxy)thiophene-2-carboxamido) piperidin-1-yl)pyrrolo[2,1-f] [1,2,4]triazin-7-yl)-2,5-dihydro- | 615.2 | E: 1.742 F: 1.520 | (400 MHZ, DMSO-d₆) δ ppm 7.90 (s, 1H), 7.38 (s, 1H), 7.22 (s, 1H), 7.10 (s, 1H), 6.97 (s, 1H), 6.76- 6.74 (m, 1H), 6.72 (d, J = 2.7 Hz, 1H), 4.58-4.49 (m, 4H), 4.33-4.27 (m, 2H), 4.14 (s, 1H), 3.66 (d, J = 4.4 Hz, 3H), 3.62 (br d, J = 4.9 Hz, 4H), 3.18 (s, |
| | | 1H-pyrrole-1-carboxylate | | | 1H), 3.02 (br s, 4H), 2.78-2.72 (m, 1H), 2.00-1.88 |
| | | | | | (m, 3H), 1.82 (br dd, J = 1.8, 4.3 Hz, 4H) |

The following examples in Table 14 were prepared using the same procedure as shown in Example 284. (R)-5-(3-aminopiperidin-1-yl)-7-(2-(difluoromethyl)pyridin-4-yl)pyrrolo[2,1-f][1,2,4]triazin-4-amine Intermediate 33 was coupled with the appropriate carboxylic acid using HATU and DIEA.

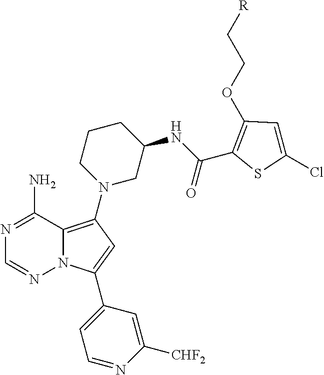

TABLE 14

| | | | | HPLC | |
|---|---|---|---|---|---|
| | | | LCMS | Method, | |
| Example | Structure (R) | Name | (M + H)⁺ | RT (min.) | ¹H NMR |
| 293 | 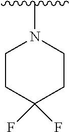 [SMILES: *N1CCC(F)(F)CC1] | (R)-N-(1-(4-amino-7-(2- (difluoromethyl)pyridin-4-yl) pyrrolo[2,1-f][1,2,4]triazin-5- yl)piperidin-3-yl)-5- chloro-3-(2-(4,4-difluoropiperidin-1- yl)ethoxy)thiophene-2-carboxamide | 667.20 | E: 2.01 F: 1.22 | (400 MHZ, DMSO-d₆) δ ppm 8.70 (d, J = 5.4 Hz, 1H), 8.46 (d, J = 1.0 Hz, 1H), 8.31-8.26 (m, 1H), 8.19 (s, 1H), 7.99 (s, 1H), 7.43 (s, 2H), 7.36 (s, 1H), 6.97 (t, J = 54.5 Hz, 2H), 4.38-4.29 (m, 2H), 4.24-4.09 (m, 1H), 3.18 (br dd, J = 3.5, 6.7 Hz, 1H), 3.01 (br d, J = 9.0 Hz, 2H), 2.93-2.70 (m, 6H), 1.97-1.77 (m, 7H), 1.56 (br s, 2H) |
| 294 | 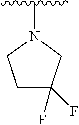 [SMILES: *N1CCC(F)(F)C1] | (R)-N-(1-(4-amino-7-(2- (difluoromethyl)pyridin-4-yl) pyrrolo[2,1-f][1,2,4]triazin-5-yl) piperidin-3-yl)-5-chloro-3-(2- (3,3-difluoropyrrolidin-1- yl)ethoxy)thiophene-2-carboxamide | 653.20 | E: 2.00 F: 1.31 | (400 MHZ, DMSO-d₆) δ ppm 8.70 (d, J = 5.1 Hz, 1H), 8.46 (s, 1H), 8.29 (d, J = 5.6 Hz, 1H), 8.24- 8.04 (m, 1H), 7.99 (s, 1H), 7.43 (s, 2H), 7.35 (s, 1H), 7.13-6.75 (m, 2H), 4.40-4.29 (m, 2H), 4.17 (br d, J = 9.3 Hz, 1H), 3.18 (br d, J = 5.1 Hz, 1H), 3.06-2.86 (m, 4H), 2.85-2.79 (m, 3H), 2.78-2.69 (m, 2H), 2.24-2.09 (m, 2H), 1.96-1.46 (m, 4H) |
| 295 | 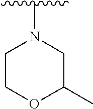 [SMILES: *N1CCOC(C)C1] | N-((R)-1-(4-amino-7-(2- (difluoromethyl)pyridin-4-yl) pyrrolo[2,1-f][1,2,4]triazin-5-yl) piperidin-3-yl)-5-chloro-3-(2-(2- methylmorpholino)ethoxy) thiophene-2-carboxamide | 647.20 | E: 1.47 F: 1.14 | (400 MHz, DMSO-d₆) δ ppm 8.70 (d, J = 5.4 Hz, 1H), 8.48 (s, 1H), 8.30 (d, J = 5.6 Hz, 1H), 8.26- 8.07 (m, 1H), 7.99 (s, 1H), 7.45 (d, J = 2.9 Hz, 2H), 7.35 (s, 1H), 7.13-6.78 (m, 2H), 4.39-4.02 (m, 3H), 3.66 (br d, J = 1.2 Hz, 1H), 3.37 (br d, J = 2.0 Hz, 1H), 3.18 (br d, J = 1.2 Hz, 2H), 3.05- 2.80 (m, 3H), 2.78-2.61 (m, 4H), 2.04-1.73 (m, |
| | | | | | 4H), 1.63 (br dd, J = 4.4, 5.6 Hz, 2H), 0.99-0.85 |
| | | | | | (m, 3H) |
| 296 | 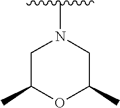 | N-((R)-1-(4-amino-7-(2- (difluoromethyl)pyridin-4-yl) pyrrolo[2,1-f][1,2,4]triazin-5-yl) piperidin-3-yl)-5-chloro-3-(2- ((25,6R)-2,6-dimethylmorpholino) ethoxy)thiophone-2-carboxamide | 661.20 | E: 1.96 F: 1.20 | (400 MHz, DMSO-d₆) δ ppm 8.70 (d, J = 5.4 Hz. 1H), 8.48 (s, 1H), 8.34-8.28 (m, 1H), 8.25-8.09 (m, 1H), 8.00 (s, 1H), 7.44 (s, 2H), 7.35(s, 1H), 7.13-6.78 (m, 2H), 4.39-4.25 (m, 2H), 4.24-4.12 (m, 1H), 3.48-3.38 (m, 2H), 3.04-2.81 (m, 3H), 2.70-2.57 (m, 5H), 1.97-1.78 (m, 3H), 1.67-1.44 (m, 3H), 0.92 (br dd, J = 6.0, 19.2 Hz, 6H) |
| 297 | 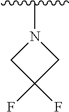 [SMILES: *N1CC(F)(F)C1] | (R)-N-(1-(4-amino-7-(2- (difluoromethyl)pyridin-4-yl) pyrrolo[2,1-f][1,2,4]triazin-5-yl) piperidin-3-yl)-5-chloro-3-(2-(3,3- difluoroazetidin-]-yl)ethoxy) thiophene-2-carboxamide | 639.10 | E: 1.94 F: 1.30 | (400 MHZ, DMSO-d₆) δ ppm 8.70 (d, J = 5.4 Hz, 1H), 8.48 (s, 1H), 8.34-8.28 (m, 1H), 8.25-8.09 (m, 1H), 8.00 (s, 1H), 7.44 (s, 2H), 7.35(s, 1H), 7.13-6.78 (m, 2H), 4.28-4.09 (m, 3H), 3.78-3.65 (m, 3H), 3.18 (s, 1H), 3.08-2.93 (m, 4H), 2.86- 2.75 (m, 1H), 1.88 (br d, J = 2.7 Hz, 3H), 1.30- 1.22 (m, 2H) |

The following examples in Table 15 were prepared using the same procedure as shown in Example 294. (R)-1-(3-(4-Amino-5-(3-aminopiperidin-1-yl)pyrrolo[2,1-f][1,2,4]triazin-7-yl)-2,5-dihydro-1H-pyrrol-1-yl)ethan-1-one (Intermediate 32) was coupled with the appropriate carboxylic acid using HATU and DIEA. Example 298 and 300 were obtained after deprotection of corresponding Boc-intermediates using excess 4N HCl in dioxane.

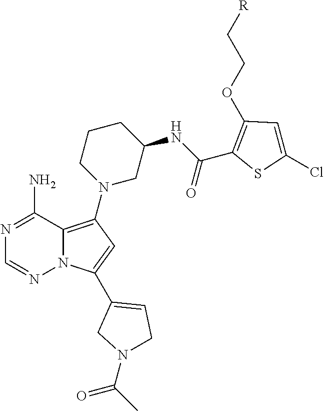

TABLE 15

| | | | | HPLC | |
|---|---|---|---|---|---|
| | | | LCMS | Method, | |
| Example | Structure (R) | Name | (M + H)⁺ | RT (min.) | ¹H NMR |
| 298 | 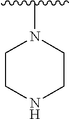 [SMILES: *N1CCNCC1] | (R)-N-(1-(7-(1-acetyl-2,5-dihydro- 1H-pyrrol-3-yl)-4- aminopyrrolo[2,1-f][1,2,4]triazin- 5-yl)piperidin-3-yl)-5-chloro-3-(2- (piperazin-1-yl)ethoxy)thiophene- 2-carboxamide | 614.20 | E: 1.18 F: 0.89 | (400 MHZ, DMSO-d₆) δ ppm 8.69-8.22 (m, 2H), 7.93 (s, 1H), 7.35 (d, J = 0.7 Hz, 2H), 7.26-6.90 (m, 1H), 6.83- 6.69 (m, 1H), 4.69 (br s,1H), 4.54-4.43 (m, 2H), 4.41- 4.26 (m, 3H), 4.21-4.13 (m, 1H), 3.16-2.96 (m, 6H), 2.88-2.64 (m, 8H), 2.04 (d, J = 15.4 Hz, 3H), 1.91-1.75 (m, 3H), 1.63-1.48 (m, 1H) |
| 299 | 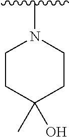 [SMILES: *N1CCC(C)(O)CC1] | (R)-N-(1-(7-(1-acetyl-2,5-dihydro- 1H-pyrrol-3-yl)-4- aminopyrrolo[2,1-f][1,2,4]triazin- 5-yl)piperidin-3-yl)-5-chloro-3-(2- (4-hydroxy-4-methylpiperidin-1- yl)ethoxy)thiophene-2- carboxamide | 643.30 | E: 1.36 F: 0.91 | (400 MHZ, DMSO-d₆) δ ppm 7.98 (br s, 1H), 7.87 (d, J = 1.7 Hz, 1H), 7.45 (s, 1H), 7.34 (s, 1H), 6.77 (br dd, J = 1.7, 12.5 Hz, 2H), 6.70 (d, J = 7.1 Hz, 1H), 4.68 (br d, J = 2.0 Hz, 1H), 4.48 (br d, J = 5.6 Hz, 2H), 4.39-4.24 (m, 3H), 4.19-4.05 (m, 2H), 3.15-2.92 (m, 3H), 2.86-2.57 (m, 5H), 2.39 (br d, J = 6.6 Hz, 1H), 2.03 (d, J = 16.4 Hz, 3H), 1.94-1.72 (m, 3H), 1.62-1.30 (m, SH), 1.01 (br d, J = 1.5 Hz, 3H) |
| 300 | 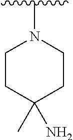 [SMILES: *N1CCC(C)(N)CC1] | (R)-N-(1-(7-(1-acetyl-2,5-dihydro- 1H-pyrrol-3-yl)-4- aminopyrrolo[2,1-f][1,2,4]triazin- 5-yl)piperidin-3-yl)-3-(2-(4- amino-4-methylpiperidin-1- yl)ethoxy)-5-chlorothiophene-2- carboxamide | 642.30 | E: 1.21 F: 0.89 | (400 MHZ, DMSO-d₆) δ ppm 7.98 (br s, 1H), 7.87 (d, J = 1.0 Hz, 1H), 7.48 (br d, J = 3.0 Hz, 1H), 7.33 (s, 1H), 6.81-6.74 (m, 2H), 6.70 (d, J = 8.8 Hz, 1H), 4.68 (br d, J = 1.0 Hz, 1H), 4.48 (br d, J = 4.8 Hz, 2H), 4.37-4.24 (m, 3H), 4.16 (br d, J = 2.5 Hz, 1H), 3.15-3.11 (m, 1H), 2.98- 2.91 (m, 1H), 2.88-2.72 (m, 2H), 2.70-2,65 (m, 2H), 2.47-2.26 (m, 4H), 2.03 (d, J = 16.0 Hz, 3H), 1.94-1.75 (m, 5H), 1.39 (br d, J = 4.0 Hz, SH), 1.02-0.92 (m, 3H) |
| 301 | 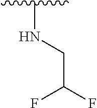 [SMILES: *NCC(F)F] | (R)-N-(1-(7-(1-acetyl-2,5-dihydro- 1H-pyrrol-3-yl)-4- aminopyrrolo[2,1-f][1,2,4]triazin- 5-yl)piperidin-3-yl)-5-chloro-3-(2- ((2,2-difluoroethyl)amino) ethoxy)thiophene-2-carboxamide | 609.20 | E: 1.53 F: 0.93 | (400 MHz, DMSO-d₆) δ ppm 7.87 (s, 1H), 7.49-7.38 (m, 1H). 7.35 (s, 1H), 6.81-6.74 (m, 1H), 6.73-6.66 (m, 1H), 6.09 (s, 1H), 4.71-4.65 (m, 1H), 4.52-4.44 (m, 2H), 4.37-4.30 (m, 2H), 4.29-4.24 (m, 1H), 4.19-4.09 (m, 1H), 3.21-3.04 (m, 4H), 3.01-2.92 (m, 1H), 2.82- 2.70 (m, 1H), 2.07-1.99 (m, 5H), 1.93-1.75 (m, 3H), 1.62-1.45 (m, 1H) |

### Example 302

### Preparation of (R)—N-(1-(4-amino-7-(4-carbamoyl-3-fluorophenyl)pyrrolo[2,1-f][1,2,4]triazin-5-yl)piperidin-3-yl)-5-(difluoromethyl)-3-(2-(dimethylamino)ethoxy)thiophene-2-carboxamide

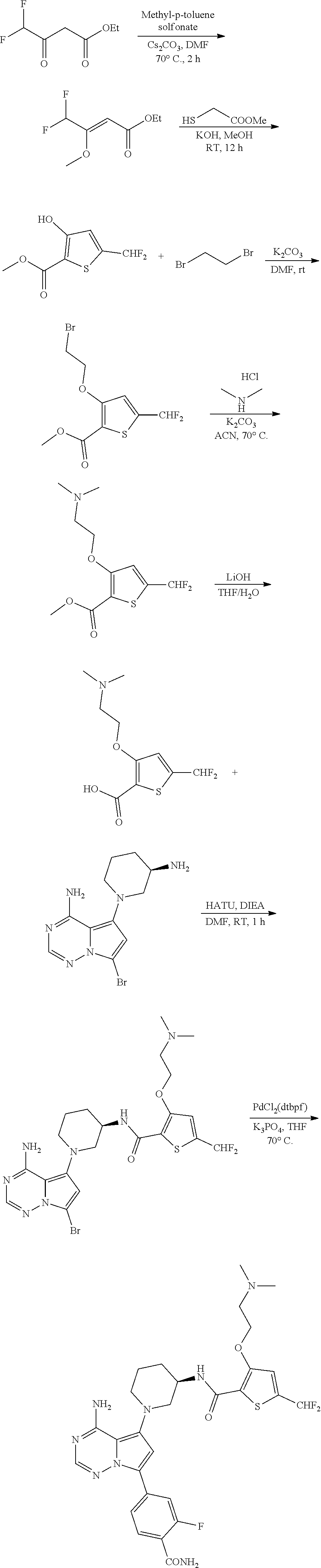

### Example 302A. (Z)-6,6-difluoro-5-methoxyhex-4-en-3-one

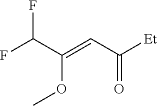

Cesium carbonate (29.4 g, 90 mmol) was added to a solution of ethyl 4,4-difluoro-3-oxobutanoate (11.81 mL, 90 mmol) in DMF (100 mL) at rt. The reaction mixture was heated to 70° C., and methyl-4-toluenesulfonate (16.40 mL, 108 mmol) in DMF (30 mL) was added dropwise for 30 min. Reaction was continued at 70° C. for 1 h. After cooling, reaction mixture was partitioned between water (500 mL) and ether (2×100 mL). Organic layer was separated, dried over sodium sulphate and concentrated under reduced pressure to afford Example 302A (130 mg, 90% yield) as red oil which was taken as such to the next step without purification.

### Example 302B. Methyl 5-(difluoromethyl)-3-hydroxythiophene-2-carboxylate

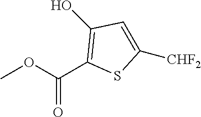

KOH (2.63 g, 46.9 mmol) in methanol (10 mL) was added dropwise to a solution of (Z)-6,6-difluoro-5-methoxyhex-4-en-3-one (6.5 g, 36.1 mmol) and methylthioglycolate (3.23 mL, 36.1 mmol) in methanol (10 mL). It was stirred at rt for 12 h. The reaction mixture was poured into ice water (100 mL) and acidified with con.H₂SO₄ till the pH 3. Extracted with ether (200 mL) and the organic layer was separated, dried over sodium sulphate and concentrated under reduced pressure. The residue was purified by flash chromatography (15% Pet.Ether/EtOAc) to afford Example 302B (5.50 g, 73% yield) as a yellow oil. MS: [M−H]⁺=207.1; ¹H NMR (400 MHz, CDCl₃) δ ppm 9.51 (s, 1H), 6.93 (s, 1H), 6.87-6.59 (t, 1H), 3.92 (s, 3H).

### Example 302C. Methyl 3-(2-bromoethoxy)-5-(difluoromethyl)thiophene-2-carboxylate

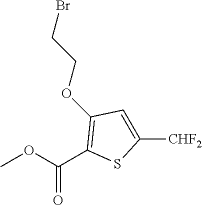 [SMILES: COC(=O)c1sc(C(F)F)cc1OCCBr]

1,2-Dibromoethane (4.16 mL, 48.0 mmol) was added dropwise to the suspension of methyl 5-(difluoromethyl)-3-hydroxythiophene-2-carboxylate (2 g, 9.61 mmol) and K₂CO₃ (1.328 g, 9.61 mmol) in DMF (30 mL). It was stirred at rt for 4 h and partitioned between water (500 mL) and ether (2×100 mL). The organic layer was separated, dried over sodium sulphate and concentrated under reduced pressure. The residue was purified by flash chromatography (30% Pet.Ether/EtOAc) to afford Example 302C (1.0 g, 33% yield) as an off-white solid. ¹H NMR (400 MHz, CDCl₃) δ ppm 7.04 (s, 1H), 6.87-6.59 (t, 1H), 4.45-4.42 (t, 2H) 3.86 (s, 3H), 3.67-3.63 (t, 2H).

### Example 302D. Methyl 5-(difluoromethyl)-3-(2-(dimethylamino)ethoxy)thiophene-2-carboxylate

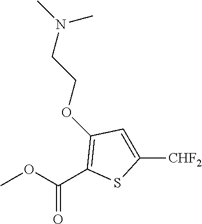 [SMILES: COC(=O)c1sc(C(F)F)cc1OCCN(C)C]

K₂CO₃ (658 mg, 4.76 mmol) was added to a solution of methyl 3-(2-bromoethoxy)-5-(difluoromethyl)thiophene-2-carboxylate (500 mg, 1.58 mmol) and dimethylamine hydrochloride (259 mg, 3.17 mmol) in acetonitrile (6 mL). Stirred at 70° C. for 9 h and partitioned between water (10 mL) and ethyl acetate (50 mL). The organic layer was separated, dried over sodium sulphate and concentrated under reduced pressure to afford Example 302D (400 mg, 90% yield) as a yellow oil. MS: [M+H]⁺=280.2; ¹H NMR (400 MHz, CDCl₃) δ ppm 7.04 (s, 1H), 6.86-6.58 (t, 1H), 4.24-4.21 (t, 2H), 3.84 (s, 3H), 2.78-2.75 (t, 2H), 2.35 (s, 6H).

### Example 302E. 5-(difluoromethyl)-3-(2-(dimethylamino)ethoxy)thiophene-2-carboxylic acid

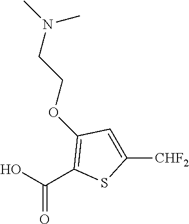 [SMILES: CN(C)CCOc1cc(C(F)F)sc1C(=O)O]

LiOH (68.6 mg, 2.86 mmol) was added to a solution of methyl 5-(difluoromethyl)-3-(2-(dimethylamino)ethoxy)thiophene-2-carboxylate (400 mg, 1.432 mmol) in a mixture of THF (4 mL)-Water (4 mL). It was stirred at rt for 12 h and acidified with 1N HCl till the pH 5. Upon evaporated to dryness, Example 302E (350 mg, 92% yield) was afforded as an off-white solid. MS: [M+H]⁺=266.1.

### Example 302F. (R)—N-(1-(4-amino-7-bromopyrrolo[2,1-f][1,2,4]triazin-5-yl)piperidin-3-yl)-5-(difluoromethyl)-3-(2-(dimethylamino)ethoxy)thiophene-2-carboxamide

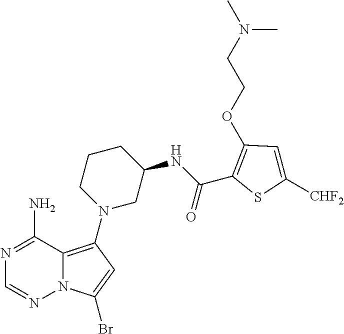 [SMILES: CN(C)CCOc1cc(C(F)F)sc1C(=O)N[C@@H]1CCCN(c2cc(Br)n3ncnc(N)c23)C1]

DIEA (0.898 mL, 5.14 mmol) was added to the suspension of (R)-5-(3-aminopiperidin-1-yl)-7-bromopyrrolo[2,1-f][1,2,4]triazin-4-amine (400 mg, 1.28 mmol), 5-(difluoromethyl)-3-(2-(dimethylamino)ethoxy)thiophene-2-carboxylic acid (341 mg, 1.28 mmol) and HATU (489 mg, 1.28 mmol) in DMF (10 mL). It was stirred at rt for 2 h and partitioned between water (10 mL) and ethyl acetate (50 mL). The organic layer was separated, dried over sodium sulphate and concentrated under reduced pressure. The resultant residue was purified by flash chromatography (8% DCM/MeOH) to afford Example 302F (400 mg, 56% yield) as a yellow solid. MS: [M+H]⁺=560.3.

### Example 302

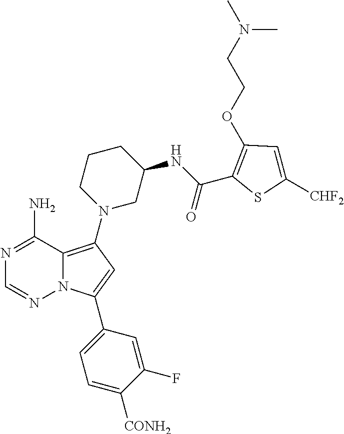 [SMILES: CN(C)CCOc1cc(C(F)F)sc1C(=O)N[C@@H]1CCCN(c2cc(-c3ccc(C(N)=O)c(F)c3)n3ncnc(N)c23)C1]

PdCl₂(dppf)-DCM adduct (3.66 mg, 4.48 μmol) was added to a solution of 2-fluoro-4-(4,4,5,5-tetramethyl-1,3,2-dioxaborolan-2-yl)benzamide (35.6 mg, 0.134 mmol), Example 302F (0.134 mL, 0.269 mmol) in THF (2 mL). The reaction mixture was heated to 75° C. for 6 h and filtered. The filtrate was concentrated and purified by preparative HPLC to afford Example 302 (28.5 mg, 52% yield). LC-MS Method E: RT=1.53 min, [M+H]⁺=617.2; LC-MS Method F: RT=0.90 min, [M+H]⁺=617.2; ¹H-NMR (400 MHz, DMSO-d₆) δ ppm 9.91-9.72 (m, 1H), 8.12 (dd, J=1.6, 12.8 Hz, 1H), 8.00 (dd, J=1.7, 8.1 Hz, 1H), 7.96 (s, 1H), 7.70-7.60 (m, 2H), 7.56 (s, 1H), 7.23 (d, J=1.5 Hz, 2H), 7.46-7.13 (m, 1H), 7.11 (s, 1H), 6.98 (s, 1H), 4.60 (t, J=4.9 Hz, 2H), 4.23-4.13 (m, 1H), 3.57 (br dd, J=2.2, 7.3 Hz, 2H), 3.27 (br d, J=7.8 Hz, 1H), 3.09-3.02 (m, 1H), 2.87-2.20 (m, 8H), 1.85 (td, J=1.5, 3.3 Hz, 3H), 1.67-1.53 (m, 1H).

The following Examples in Table 16 were prepared using the same procedure as shown in Example 302. Example 302F was coupled with the appropriate boronic acid or boronate ester, using potassium phosphate tribasic monohydrate (2M aq. soln.) and [1,1′-bis(di-tert-butylphosphino)ferrocene]dichloropalladium(II)] in THF at 75° C. for 6 h.

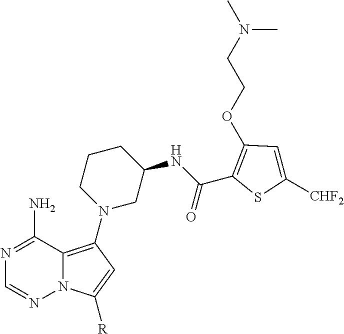

TABLE 16

| | | | | HPLC | |
|---|---|---|---|---|---|
| | | | | Method, | |
| Ex- | Structure | | LCMS | RT | |
| ample | (R) | Name | (M + H)⁺ | (min.) | ¹H NMR |
| 303 | 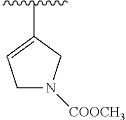 [SMILES: *C1=CCN(C(=O)OC)C1] | Methyl (R)-3-(4- amino-5-(3-(5- (difluoromethyl)- 3-(2- (dimethylamino) ethoxy) thiophene-2- carboxamido) piperidin-1 - yl)pyrrolo [2,1-f][1,2,4] triazin-7-yl)- 2,5-dihydro-1H- pyrrole-1- carboxylate | 605.20 | E: 1.65 F: 1.01 | (400 MHz, DMSO-d6) δ ppm 7.97 (bs, 1H), 7.86 (s, 2H), 7.55 (s, 1H), 7.25 (t, J = 55.8 Hz, 1H), 6.84 (bs, 1H), 6.78-6.69 (m, 2H), 4.53 (br d, J = 13.0 Hz, 2H), 4.37-4.26 (m, 4H), 4.17 (br s, 1H), 3.66 (d, J = 4.2 Hz, 3H), 3.14 (br d, J = 7.6 Hz, 1H), 3.02-2.93 (m, 1H), 2.86-2.69 (m, 2H), 2.65-2.53 (m, 2H), 2.19 (br s, 6H), 1.92 (s, 3H), 1.62-1.41 (m, 1H) |
| 304 | 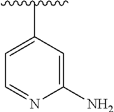 [SMILES: *c1ccnc(N)c1] | (R)-N-(1-(4- amino-7-(2- aminopyridin- 4-yl)pyrrolo [2,1-f][1,2,4] triazin-5- yl)piperidin-3-yl)-5- (difluoromethyl)- 3-(2- (dimethylamino) ethoxy) thiophene-2- carboxamide | 572.20 | E: 1.43 F: 0.80 | (400 MHz, DMSO-d₆) δ ppm 9.21 (bs, 2H), 7.92 (d,) = 5.4 Hz, 1H), 7.90-7.85 (m, 2H), 7.58-7.51 (m, 1H), 7.41- 7.22 (m, 1H), 7.14-7.05 (m, 2H), 6.92 (bs, 1H), 5.91(s, 2H), 4.33 (t, J = 5.3 Hz, 2H), 4.19 (br d, J = 0.7 Hz, 1H), 3.18 (br d, J = 3.7 Hz, 1H), 3.01 (br d, j = 1.2 Hz, 1H), 2.81 (s, 1H), 2.63-2.56 (m, 2H), 2.18 (s, 6H), 2.07 (s, 3H), 1.96-1.81 (m, 3H), 1.62-1.41 (m, 1H) |
| 305 | 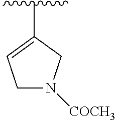 [SMILES: *C1=CCN(C(C)=O)C1] | (R)-N-(1-(7-(1- acetyl-2,5- dihydro-1H- pyrrol-3-yl)-4- aminopyrrolo[2,1- f][1,2,4]triazin-5-yl) piperidin-3-yl)-5- (difluoromethyl)-3- (2-(dimethylamino) ethoxy)thiophene-2- carboxamide | 589.20 | E: 1.44 F: 0.89 | (400 MHz, DMSO-d6) δ ppm 8.07-7.95 (m, 1H), 7.86 (s, 1H), 7.84-7.74 (m, 1H), 7.54 (d, J = 1.3 Hz, 1H), 7.25 (t, J = 55.0 Hz, 1H), 6.90 (bs, 1H), 6.80-6.65 (m, 2H), 4.71- 4.64 (m, 1H), 4.54-4.30 (m, 5H), 4.26 (br d, J = 2.8 Hz, 1H), 4.22-4.03 (m, 1H), 3.18-3.11 (m, 1H), 3.02-2.93 (m, 1H), 2.86-2.61 (m, 3H), 2.40-2.13 (m, 6H), 2.02 (d,) = 16.0 Hz, 3H), 1.94-1.68 (m, 3H), 1.58-1.42 (m, 1H) |
| 306 | 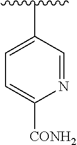 [SMILES: *c1ccc(C(N)=O)nc1] | (R)-5-(4-amino-5- (3-(5- (difluoromethyl)-3- (2-(dimethylamino) ethoxy)thiophene-2- carboxamido) piperidin-1- yl)pyrrolo[2,1-f] [1,2,4]triazin-7- yl)picolinamide | 600.20 | E: 1.46 F: 0.88 | (400 MHz, DMSO-d₆) δ ppm 9.33-9.26 (m, 1H), 8.68 (dd, J = 2.3, 8.5 Hz, 1H), 8.17-8.04 (m, 2H), 7.92 (s, 2H), 7.64 (br s, 1H), 7.56 (s, 1H), 7.41-7.06 (m, 2H), 7.01-6.89 (m, 1H), 6.52 (s, 1H), 4.36 (br s, 2H), 4.25-4.17 (m, 1H), 3.25-3.17 (m, 1H), 3.08-2.98 (m, 1H), 2.91-2.68 (m, 5H), 2.23 (br s, 6H), 1.97-1.76 (m, 2H), 1.62-1.41 (m, 1H) |
| 307 | 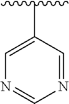 [SMILES: *c1cncnc1] | (R)-N-(1-(4-amino-7- (pyrimidin-5- yl)pyrrolo[2,1-f] [1,2,4]triazin-5- yl)piperidin-3-yl)-5- (difluoromethyl)- 3-(2- (dimethylamino) ethoxy)thiophene-2- carboxamide | 558.20 | E: 1.52 F: 0.89 | (400 MHz, DMSO-d₆) δ ppm 9.47 (s, 2H), 9.09 (s, 1H), 8.16 (br s, 1H), 7.92 (s, 2H), 7.56 (s, 1H), 7.32 (s, 1H), 7.26 (t, J = 55.0 Hz, 1H), 6.91 (s, 1H), 4.35 (br t, J = 4.6 Hz, 2H), 4.23 (br s, 1H), 3.23-3.15 (m, 1H), 3.04 (br d,) = 5.9 Hz, 1H), 2.84 (br s, 2H), 2.66-2.56 (m, 2H), 2.28-2.07 (m, 6H), 1.98-1.80 (m, 3H), 1.62-1.41 (m, 1H) |
| 308 | 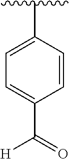 [SMILES: *c1ccc(C=O)cc1] | (R)-N-(1-(4-amino-7- (4-formylphenyl) pyrrolo[2,1- f][1,2,4]triazin-5-yl) piperidin-3-yl)-5- (difluoromethyl)-3- (2-(dimethylamino) ethoxy)thiophene-2- carboxamide | 584.20 | E: 1.95 F: 1.17 | (400 MHz, DMSO-d₆) δ ppm 10.01 (s, 1H), 8.37 (d, J = 8.3 Hz, 2H), 8.15 (bs, 1H), 7.97 (d, J = 8.6 Hz, 2H), 7.93- 7.85 (m, 2H), 7.56 (s, 1H), 7.27 (s, 1H), 7.26 (t, J = 59.4 Hz, 1H), 6.91 (bs, 1H), 4.39-4.29 (m, 2H), 4.21 (br s, 1H), 3.91 (s, 2H), 3.25-3.14 (m, 1H), 3.04 (br s, 2H), 2,65-2.57 (m, 2H), 2.19 (br s, 6H), 1.90 (m, 2H), 1.54 (m, 1H) |
| 309 | 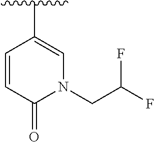 [SMILES: *c1ccc(=O)n(CC(F)F)c1] | (R)-N-(1-(4-amino-7- (1-(2,2- difluoroethyl)-6-oxo- 1,6-dihydropyridin-3- yl)pyrrolo[2,1- f][1,2,4]triazin-5-yl) piperidin-3-yl)-5- (difluoromethyl)-3- (2-(dimethylamino) ethoxy)thiophene-2- carboxamide | 637.20 | E: 1.64 F: 1.03 | (400 MHz, DMSO-d₆) δ ppm 9.73 (s, 1H), 8.50 (d, j = 2.5 Hz, 1H), 8.09 (dd, J = 2.5, 9.5 Hz, 1H), 7.87 (s, 1H), 7.55 (s, 1H), 7.44-7.13 (m, 2H), 7.10-6.94 (m, 1H), 6.91 (s, 1H), 6.58 (d, J = 9.5 Hz, 1H), 6.52-6.21 (m, 1H), 4.59 (t, J = 5.0 Hz, 2H), 4.50-4.40 (m, 2H), 4.19 (br s, 1H), 3.58-3.55 (m, 2H), 3.27-3.24 (m, 1H), 3.07-2.95 (m, 2H), 2.86 (br s, 6H), 2.77 (br s, 2H), 1.97-1.77 (m, 2H), 1.62- 1.41 (m, 1H) |
| 310 | 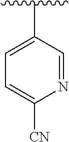 [SMILES: *c1ccc(C#N)nc1] | (R)-N-(1-(4-amino-7- (6-cyanopyridin- 3-yl)pyrrolo[2,1-f] [1,2,4]triazin-5- yl)piperidin-3-yl)-5- (difluoromethyl)- 3-(2- (dimethylamino) ethoxy)thiophene-2- carboxamide | 582.20 | E: 1.78 F: 1.07 | (400 MHz, DMSO-d6) δ ppm 9.44 (d, J = 2.2 Hz, 1H), 8.81 (dd, J = 2.3, 8.4 Hz, 1H), 8.09 (d, J = 8.3 Hz, 1H), 8.20 (bs, 1H), 7.96 (s, 1H), 7.92-7.86 (m, 1H), 7.55 (s, 1H), 7.43 (s, 1H), 7.25 (t, J = 56.2 Hz, 1H), 6.97 (br s, 1H), 4.34 (t, J = 5.4 Hz, 2H), 4.26-4.18 (m, 1H), 3.23-3.16 (m, 1H), 3.08-3.01 (m, 1H), 2.91-2.74 (m, 2H), 2.61 (br d, J = 6.1 Hz, 2H), 2.19 (s, 6H), 1.97-1.77 (m, 3H), 1.62-1.41(m, 1H) |
| 312 | 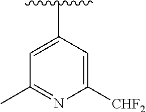 [SMILES: *c1cc(C)nc(C(F)F)c1] | (R)-N-(1-(4-amino- 7-(2-(difluoromethyl)- 6-methylpyridin-4- yl)pyrrolo[2,1-f] [1,2,4]triazin-5- yl)piperidin-3-yl)-5- (difluoromethyl)- 3-(2- (dimethylamino) ethoxy)thiophene-2- carboxamide | 621.20 | E: 1.92 F: 1.21 | (400 MHz, DMSO-d6) δ ppm 8.31 (s, 1H), 8.15 (s, 1H), 7.98 (s, 1H), 7.74 (s, 1H), 7.55 (s, 1H), 7.42 (s, 1H), 7.26 (t, J = 56.5 Hz, 1H), 6.91 (t, J = 55.5 Hz, 1H), 4.41 (br., s, 2H), 4.20 (br s, 1H), 3.24 (br., s, 2H), 3.09 - 2.99 (m, 1H), 2.82 (br., s, 3H), 2.55 (s, 4H), 2.44-2.26 (m, 4H), 1.87 (br., s, 3H), 1.55 (br., s, 1H), 1.23 (s, 4H) |
| 313 | 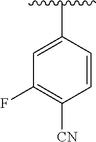 [SMILES: *c1ccc(C#N)c(F)c1] | (R)-N-(1-(4-amino- 7-(4-cyano-3- fluorophenyl)pyrrolo [2,1- [][1,2,4]triazin-5-yl) piperidin-3-yl)-5- (difluoromethyl)-3- (2-(dimethylamino) ethoxy)thiophene-2- carboxamide | 599.20 | E: 2.04 F: 1.29 | (400 MHz, DMSO-d6) δ ppm 8.36 (dd, J= 1.4, 11.9 Hz, 1H), 8.21 (dd, J= 1.6, 8.4 Hz, 2H), 8.01 - 7.90 (m, 2H), 7.73 (br s, 1H), 7.55 (s, 1H), 7.40 - 7.09 (m, 2H), 6.96 (br., s. 1H), 4.50 - 4.30 (m, 2H), 4.20 (br.,s, 1H), 3.20 (br., s, 1H), 3.08 - 2.98 (m, 1H), 2.80 (br., s, 3H), 2.40 (br., s, 4H), 2.44-2.26 (m, 4H), 1.86 (br., s, 3H), 1.53 (br., s, 1H), 1.23 (s, 4H) |

### Example 314

### Preparation of (R)—N-(1-(4-amino-7-(pyrimidin-5-yl)pyrrolo[2,1-f][1,2,4]triazin-5-yl)piperidin-3-yl)-5-(difluoromethyl)-3-(2-oxa-6-azaspiro[3.3]heptan-6-yl)thiophene-2-carboxamide

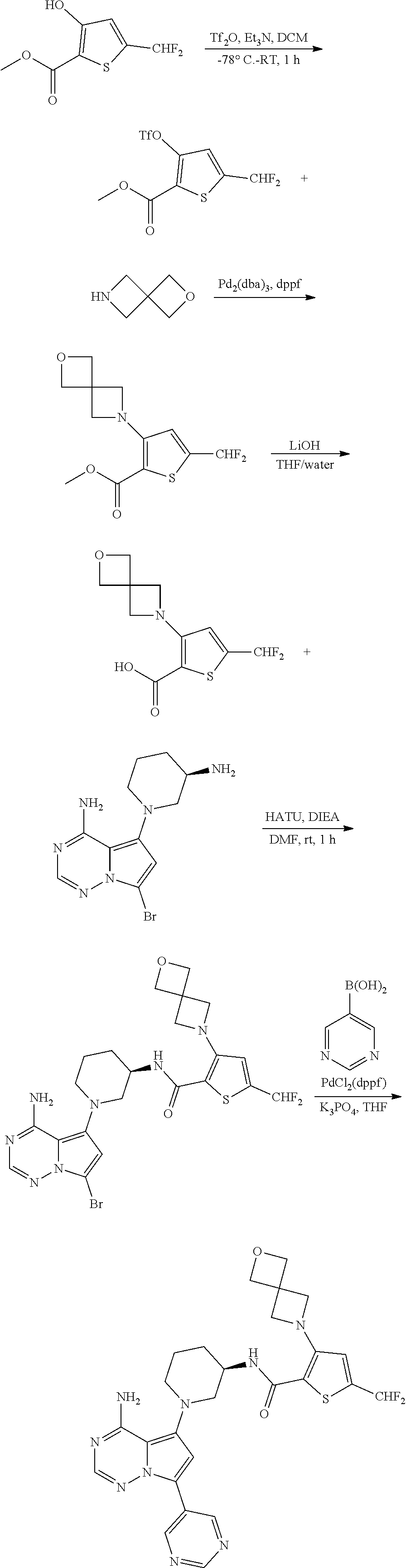

### Example 314A. Methyl 5-(difluoromethyl)-3-(((trifluoromethyl)sulfonyl)oxy)thiophene-2-carboxylate

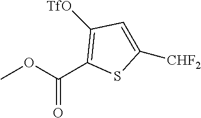 [SMILES: COC(=O)c1sc(C(F)F)cc1OS(=O)(=O)C(F)(F)F]

Triflic anhydride (1.582 mL, 9.37 mmol) was added dropwise to a solution of methyl 5-(difluoromethyl)-3-hydroxythiophene-2-carboxylate (1.5 g, 7.21 mmol) and triethylamine (2.009 mL, 14.41 mmol) in DCM (30 mL) at −78° C. The reaction mixture was stirred at −78° C. for 1 h, followed by 1 h at rt. The mixture was partitioned between water (100 mL) and chloroform (100 mL). The organic layer was separated, dried over sodium sulphate and concentrated under reduced pressure. The resultant residue was purified by flash chromatography (10% Pet.Ether/EtOAc) to afford Example 314A (2000 mg, 82% yield) as a yellow oil. MS: [M+H]⁺=341.3; ¹H NMR (300 MHz, CDCl₃) δ ppm 7.19 (s, 1H), 6.92-6.64 (t, 1H), 3.95 (s, 3H).

### Example 314B. Methyl 5-(difluoromethyl)-3-(2-oxa-6-azaspiro[3.3]heptan-6-yl)thiophene-2-carboxylate

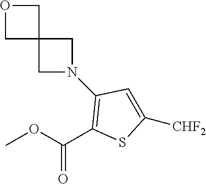 [SMILES: COC(=O)c1sc(C(F)F)cc1N1CC2(COC2)C1]

Argon was purged through the suspension of Example 314A (3 g, 8.82 mmol), 2-oxa-6-azaspiro[3.3]heptane (1.311 g, 13.23 mmol), dppf (0.489 g, 0.882 mmol) and cesium carbonate (5.75 g, 17.63 mmol) in toluene (50 mL). Pd₂(dba)₃ (0.404 g, 0.441 mmol) was added and heated at 110° C. for 12 h. The reaction mixture was partitioned between water (100 mL) and ethyl acetate (100 mL), organic layer was separated, dried over sodium sulphate and concentrated under reduced pressure. The resultant residue was purified by flash chromatography (10% DCM/MeOH) to afford Example 314B (1400 mg, 55% yield) as a yellow solid. MS: [M+H]⁺=290.2; ¹H NMR (300 MHz, CDCl₃) δ ppm 6.61 (s, 1H), 6.83-6.55 (t, 1H), 4.81 (s, 4H), 4.40 (s, 4H), 3.81 (s, 3H).

### Example 314C. 5-(difluoromethyl)-3-(2-oxa-6-azaspiro[3.3]heptan-6-yl)thiophene-2-carboxylic acid, lithium salt

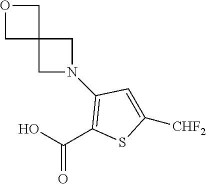 [SMILES: O=C(O)c1sc(C(F)F)cc1N1CC2(COC2)C1]

LiOH (116 mg, 4.84 mmol) was added to a solution of Example 314B (700 mg, 2.420 mmol) in a mixture of THF (7 mL)-Water (7 mL) and stirred at rt for 4 h. It was evaporated to dryness and azeotroped with acetonitrile to afford Example 314C (600 mg, 90% yield) as a pale brown solid (Lithium salt). MS: [M+H]⁺=276.2; ¹H NMR (300 MHz, DMSO-d₆) δ ppm 6.62 (s, 1H), 6.62-6.73 (t, 1H), 4.65 (s, 4H), 4.02 (s, 4H).

### Example 314D. (R)—N-(1-(4-amino-7-bromopyrrolo[2,1-f][1,2,4]triazin-5-yl)piperidin-3-yl)-5-(difluoromethyl)-3-(2-oxa-6-azaspiro[3.3]heptan-6-yl)thiophene-2-carboxamide

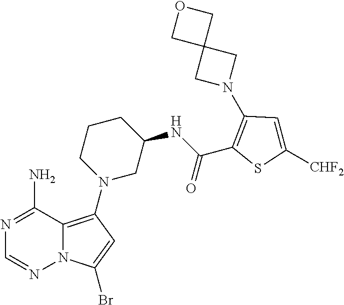 [SMILES: Nc1ncnn2c(Br)cc(N3CCC[C@@H](NC(=O)c4sc(C(F)F)cc4N4CC5(COC5)C4)C3)c12]

DIEA (1.010 mL, 5.78 mmol) was added to a solution of (R)-5-(3-aminopiperidin-1-yl)-7-bromopyrrolo[2,1-f][1,2,4]triazin-4-amine (600 mg, 1.928 mmol), Example 314C (531 mg, 1.928 mmol) and HATU (733 mg, 1.928 mmol) in DMF (10 mL). Stirred at rt for 1 h and partitioned between water (50 mL) and ethyl acetate (50 mL). The organic layer was separated, dried over sodium sulphate and concentrated under reduced pressure. The residue was purified by flash chromatography (8% DCM/MeOH) to afford Example 314D (400 mg, 55.7% yield) as a yellow solid. MS: [M+H]⁺=570.2.

### Examples 314

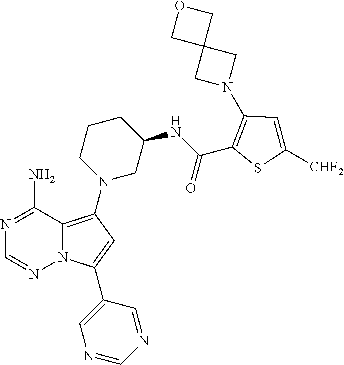 [SMILES: Nc1ncnn2c(-c3cncnc3)cc(N3CCC[C@@H](NC(=O)c4sc(C(F)F)cc4N4CC5(COC5)C4)C3)c12]

PdCl₂(dppf)-DCM adduct (7.18 mg, 8.80 μmol) was added to a solution of Example 314D (50 mg, 0.088 mmol), pyrimidin-5-ylboronic acid (21.80 mg, 0.176 mmol) and tripotassium phosphate (0.132 mL, 0.264 mmol) in THF (2 mL). The reaction mixture was heated to 75° C. for 6 h. After cooling, mixture was filtered, concentrated and purified by preparative HPLC to afford Example 314 (R)—N-(1-(4-amino-7-(pyrimidin-5-yl)pyrrolo[2,1-f][1,2,4]triazin-5-yl)piperidin-3-yl)-5-(difluoromethyl)-3-(2-oxa-6-azaspiro[3.3]heptan-6-yl)thiophene-2-carboxamide (6 mg, 11% yield). LC-MS Method E: RT=1.46 min, [M+H]⁺=568.2; LC-MS Method F: RT=1.22 min, [M+H]⁺=568.2; ¹H-NMR (400 MHz, DMSO-d₆) δ ppm 9.47 (s, 2H), 9.08 (s, 1H), 8.13 (br s, 1H), 7.95-7.88 (m, 2H), 7.35-7.04 (m, 2H), 6.99 (s, 2H), 4.68-4.54 (m, 4H), 4.17-4.10 (m, 1H), 4.03 (s, 4H), 3.23-3.19 (m, 1H), 2.97 (br s, 1H), 2.85-2.68 (m, 2H), 1.95-1.71 (m, 3H), 1.62-1.41 (m, 1H).

The following examples in Table 17 were prepared using the same procedure as shown in Example 314. Example 314D was coupled with the appropriate boronic acid or boronate ester, using potassium phosphate tribasic monohydrate (2M aq. soln.) and [1,1′-bis(di-tert-butylphosphino)ferroecene]dichloropalladium(II)] in THF at 75° C. for 6 h.

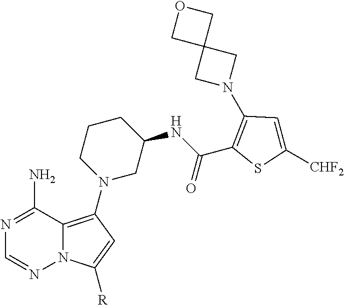

TABLE 17

| | | | | HPLC | |
|---|---|---|---|---|---|
| Ex- | Structure | | LCMS | Method, | |
| ample | (R) | Name | (M + H)⁺ | RT (min.) | ¹H NMR |
| 315 | 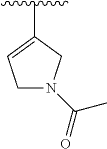 [SMILES: *C1=CCN(C(C)=O)C1] | (R)-N-(1-(7-(1-acetyl-2,5- dihydro-1H-pyrrol-3-yl)-4- aminopyrrolo[2,1- f][1,2,4]triazin-5- yl)piperidin-3-yl)-5- (difluoromethyl)-3-(2-oxa-6- azaspiro[3.3]heptan-6- yl)thiophene-2-carboxamide | 599.20 | E: 1.40 F: 1.22 | (400 MHz, DMSO-d₆) δ ppm 8.77 (s, 1H), 8.02 (bs, 1H), 7.87 (d, J = 2.0 Hz, 2H), 7.36-7.16 (m, 1H), 7.09-6.97 (m, 1H), 6.93- 6.67 (m, 2H), 4.74-4.57 (m, 4H), 4.48 (td, J = 1.4, 12.2 Hz, 2H), 4.30-4.24 (m, 1H), 4.15-3.97 (m, 4H), 3.20-3.11 (m, 2H), 2.93 (br s, 1H), 2.67 (br d, J = 1,8 Hz, 2H), 2.18-2.07 (m, 1H), 2.03 (d, J = 16.8 Hz, 3H), 1.92-1.66 (m, 3H), 1.62-1.41 (m, 1H) |
| 316 | 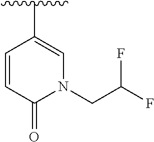 [SMILES: *c1ccc(=O)n(CC(F)F)c1] | (R)-N-(1-(4-amino-7-(1-(2,2- difluoroethyl)-6-oxo-1,6- dihydropyridin-3- yl)pyrrolo[2,1- f][1,2,4]triazin-5- yl)piperidin-3-yl)-5- (difluoromethyl)-3-(2-oxa-6- azaspiro[3.3]heptan-6- yl)thiophene-2-carboxamide | 647.20 | E: 1.56 F: 1.31 | (400 MHz, DMSO-d6) δ ppm 8.51 (s, 1H), 8.13 (dd, J = 2.1, 9.6 Hz, 1H), 8.05 (bs, 1H), 8.00-7.74 (m, 3H), 7.20 (t, J = 56.0 Hz, 1H), 7.00 (s, 1H), 6.92 (s, 1H), 6.57 (d, J = 9.3 Hz, 1H), 6.53- 6.18 (m, 1H), 4.70-4.55 (m, 4H), 4.50-4.42 (m, 2H), 4.15-3.99 (m, 5H), 3.20-3.17 (m, 1H), 3.01-2.91 (m, 1H), 2.84-2.69 (m, 2H), 1.96-1.71 (m, 3H), 1.54 (br s, 1H) |
| 317 | 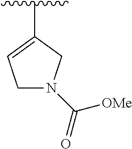 [SMILES: *C1=CCN(C(=O)OC)C1] | Methyl (R)-3-(4-amino-5-(3- (5-(difluoromethyl)-3-(2- oxa-6-azaspiro[3.3]heptan-6- yl)thiophene-2- carboxamido)piperidin-1- yl)pyrrolo[2,1- f][1,2,4]triazin-7-yl)-2,5- dihydro-1H-pyrrole-1- carboxylate | 615.20 | E: 1.72 F: 1.39 | (400 MHz, DMSO- d₆) δ ppm 8.53 (s, 1H), 7.96 (s, 1H), 7.88 (br dd, J = 0.8, 7.0 Hz, 1H), 7.20 (t, J = 54.5 Hz, 2H), 7.00 (s, 1H), 6.83 (d, J = 6.5 Hz, 1H), 6.78 (br d, J = 1.3 Hz, 1H), 4.68- 4.60 (m, 4H), 4.59-4.52 (m, 2H), 4.40 - 4.31 (m, J = 6.0, 8.0 Hz, 2H), 4.16-4.08 (m, 1H), 4.04 (s, 4H), 3.66 (d, J = 4.0 Hz, 3H), 3.19-3.17 (m, 1H), 3.00-2.91 (m, 1H), 2.79-2.68 (m, 2H), 1.92- 1.72 (m, 3H), 1.60-1.49 (m, 1H) |

### Example 318

### Preparation of (R)—N-(1-(4-amino-7-(6-cyanopyridin-3-yl)pyrrolo[2,1-f][1,2,4]triazin-5-yl)piperidin-3-yl)-5-(difluoromethyl)-3-morpholinothiophene-2-carboxamide

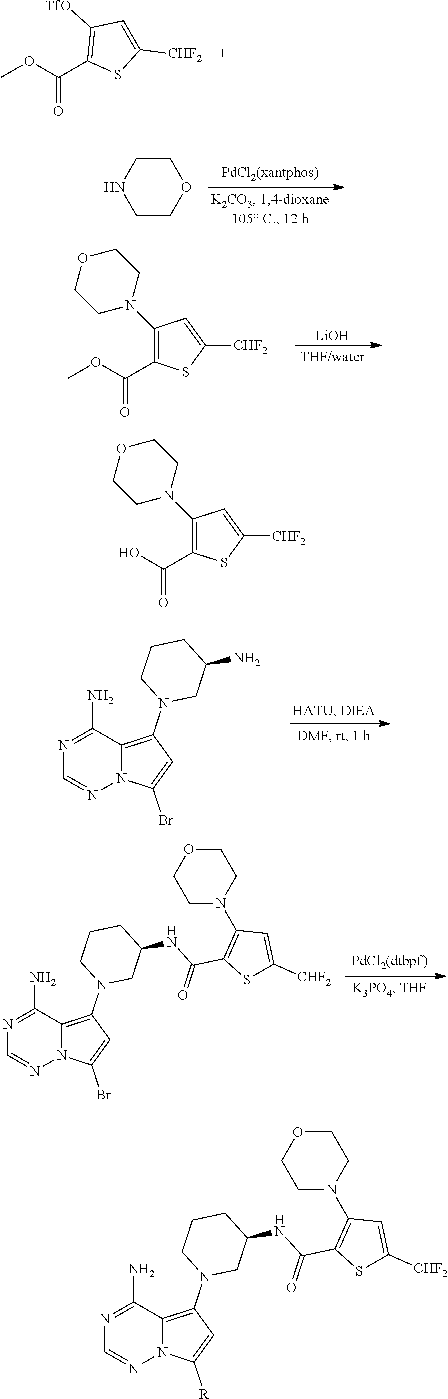

### Example 318A. Methyl 5-(difluoromethyl)-3-morpholinothiophene-2-carboxylate

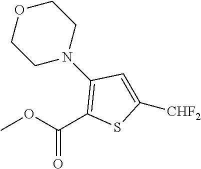 [SMILES: COC(=O)c1sc(C(F)F)cc1N1CCOCC1]

Dichloro[9,9-Dimethyl-4,5-bis(diphenylphosphino)xanthene]Palladium(II) (0.444 g, 0.588 mmol) was added to a suspension of methyl 5-(difluoromethyl)-3-(((trifluoromethyl)sulfonyl)oxy)thiophene-2-carboxylate (2 g, 5.88 mmol), morpholine (0.768 g, 8.82 mmol) and K₂CO₃ (1.625 g, 11.76 mmol) in dioxane (40 mL). The reaction mixture was heated at 105° C. for 12 h. Cooled and partitioned between water (50 mL) and ethyl acetate (50 mL). The organic layer was separated, dried over sodium sulphate and concentrated under reduced pressure. The resultant residue was purified by flash chromatography (8% DCM/MeOH) to afford Example 318A (1000 mg, 61% yield) as a yellow semisolid. MS: [M+H]⁺=570.2.

### Example 318B. 5-(difluoromethyl)-3-morpholinothiophene-2-carboxylic acid

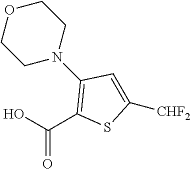 [SMILES: O=C(O)c1sc(C(F)F)cc1N1CCOCC1]

Analogous to the procedure for the preparation of Example 314C, reaction of methyl 5-(difluoromethyl)-3-morpholinothiophene-2-carboxylate (1 g, 3.61 mmol) and LiOH (0.173 g, 7.21 mmol) afforded Example 318B (900 mg, 95% yield) as a yellow solid. MS: [M+H]⁺=264.2.

### Example 318C. (R)—N-(1-(4-amino-7-bromopyrrolo[2,1-f][1,2,4]triazin-5-yl)piperidin-3-yl)-5-(difluoromethyl)-3-morpholinothiophene-2-carboxamide

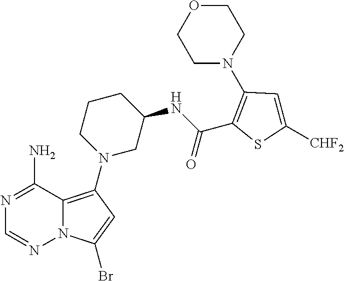 [SMILES: Nc1ncnn2c(Br)cc(N3CCC[C@@H](NC(=O)c4sc(C(F)F)cc4N4CCOCC4)C3)c12]

Analogous the procedure for the preparation of Example 314D, reaction of 5-(difluoromethyl)-3-morpholinothiophene-2-carboxylic acid (500 mg, 1.909 mmol) and (R)-5-(3-aminopiperidin-1-yl)-7-bromopyrrolo[2,1-f][1,2,4]triazin-4-amine (591 mg, 1.90 mmol) afforded Example 318C (700 mg, 66% yield) as a yellow solid. MS: [M+H]⁺=556.0.

### Examples 318

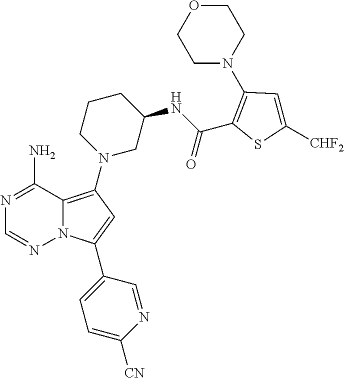 [SMILES: N#Cc1ccc(-c2cc(N3CCC[C@@H](NC(=O)c4sc(C(F)F)cc4N4CCOCC4)C3)c3c(N)ncnn23)cn1]

[1,1′-Bis(di-tert-butylphosphino)ferrocene]dichloropalladium(II)] (7.03 mg, 10.78 μmol) was added to a solution of Example 318C (60 mg, 0.108 mmol), 5-(4,4,5,5-tetramethyl-1,3,2-dioxaborolan-2-yl)picolinonitrile (37.2 mg, 0.162 mmol) and tripotassium phosphate (0.162 mL, 0.323 mmol) in THF (3 mL). The reaction mixture was heated at 75° C. for 6 h, cooled and filtered. The filtrate was concentrated and purified by preparative HPLC to afford Example 318 (12.9 mg, 20% yield). LC-MS Method E: RT=1.81 min, [M+H]⁺=580.2; LC-MS Method F: RT=1.51 min, [M+H]⁺=580.2; ¹H-NMR (400 MHz, DMSO-d₆) δ ppm 9.44 (d, J=2.0 Hz, 1H), 9.10-8.94 (m, 1H), 8.88-8.73 (m, 1H), 8.27-8.13 (m, 1H), 8.19-8.04 (m, 1H), 7.96 (s, 1H), 7.68 (s, 1H), 7.43 (s, 1H), 7.26 (s, 1H), 6.95 (bs, 1H), 4.27-4.15 (m, 1H), 3.91 (s, 1H), 3.76 (br t, J=4.3 Hz, 4H), 3.10-3.00 (m, 1H), 2.96 (br d, J=3.3 Hz, 4H), 2.89-2.65 (m, 2H), 2.07-1.96 (m, 1H), 1.94-1.84 (m, 2H), 1.65-1.49 (m, 1H).

The following Examples in Table 18 were prepared using the same procedure as shown in Example 318. Example 318C was coupled with the appropriate boronic acid or boronate ester, using potassium phosphate tribasic monohydrate (2M aq. soln.) and [1,1′-bis(di-tert-butylphosphino)ferrocene]dichloropalladium(II)] in THF at 75° C. for 6 h.

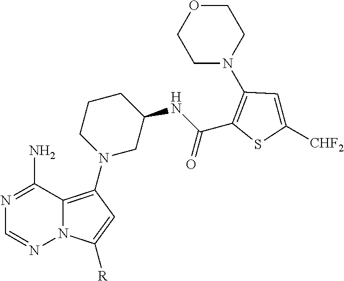

TABLE 18

| | | | | HPLC | |
|---|---|---|---|---|---|
| | Structure | | LCMS | Method, | |
| Example | (R) | Name | (M + H)⁺ | RT (min.) | ¹H NMR |
| 319 | 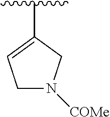 | (R)-N-(1-(7-(1-acetyl-2,5- dihydro-1H-pyrrol-3-yl)-4- aminopyrrolo[2,1- f][1,2,4]triazin-5-yl)piperidin- 3-yl)-5-(difluoromethyl)-3- morpholinothiophene-2- carboxamide | 587.20 | E: 1.51 F: 1.26 | (400 MHz, DMSO-d₆) δ ppm 9.07-8.92 (m, 1H), 8.55-8.17 (m, 1H), 8.02-7.90 (m, 1H), 7.72-7.62 (m, 1H), 7.44-7.11 (m, 1H), 7.13-7.07 (m, 1H), 7.01-6.93 (m, 1H), 6.88-6.70 (m, 1H), 4.75-4.65 (m, 1H), 4.56-4.43 (m, 2H), 4.31-4.15 (m, 2H), 3.97-3.86 (m, 1H), 3.75 (br s, 4H), 3.10-2.91 (m, 5H), 2.87-2.70 (m, 2H), 2.01 (s, 3H), 1.92-1.82 (m, 3H), 1.64-1,47 (m, 1H) |
| 320 | 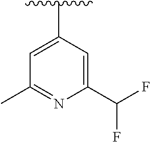 [SMILES: *c1cc(C)nc(C(F)F)c1] | Methyl (R)-3-(4-amino-5-(3- (5-(difluoromethyl)-3- morpholinothiophene-2- carboxamido)piperidin-1- yl)pyrrolo[2,1-f][1,2,4]triazin- 7-yl)-2,5-dihydro-1H-pyrrole- 1-carboxylate | 619.20 | E: 1.92 F: 1.61 | (400 MHz, DMSO-d₆) δ ppm 9.03 (br d, J = 6.8 Hz, 1H), 8.32 (s, 1H), 8.15 (s, 2H), 7.98 (s, 1H), 7.67 (s, 1H), 7.44 (s, 1H), 7.41-7.09 (m, 1H), 7.07-6.66 (m, 2H), 4.24-4.16 (m, 1H), 3.75 (br t, J = 4.3 Hz, 4H), 3.09-3.00 (m, 1H), 2.99-2.90 (m, 4H), 2.84 (br s, 2H), 2.55 (s, 3H), 1.99 (br s, 1H), 1.93-1.81 (m, 3H), 1.57 (br s, 1H) |
| 321 | 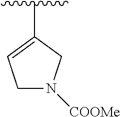 [SMILES: *C1=CCN(C(=O)OC)C1] | Methyl (R)-3-(4-amino-5-(3- (5-(difluoromethyl)-3- morpholinothiophene-2- carboxamido)piperidin-1- yl)pyrrolo[2,1-f][1,2,4]triazin- 7-yl)-2,5-dihydro-1H-pyrrole- 1-carboxylate | 603.20 | E: 1.76 F: 1.45 | (400 MHz, DMSO-d₆) δ ppm 9.06-8.91 (m, 1H), 7.95 (s, 1H), 7.68 (s, 1H), 7.47-7.02 (m, 2H), 6.89-6.79 (m, 1H), 6.78-6.72 (m, 2H), 4.63-4.49 (m, 2H), 4.37-4.25 (m, 1H), 4.36-4.08 (m, 4H), 3.75 (br s, 3H), 3.66 (brd, J = 3.5 Hz, 4H), 2.95 (brd, J = 3.3 Hz, 4H), 2.86-2.68 (m, 2H), 2.07- 1.94 (m, 1H), 1.92-1,80 (m, 2H), 1.65-1.44 (m, 1H) |

### Example 322

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(1-methyl-1,2,3,6-tetrahydropyridin-4-yl)thiophene-2-carboxamide

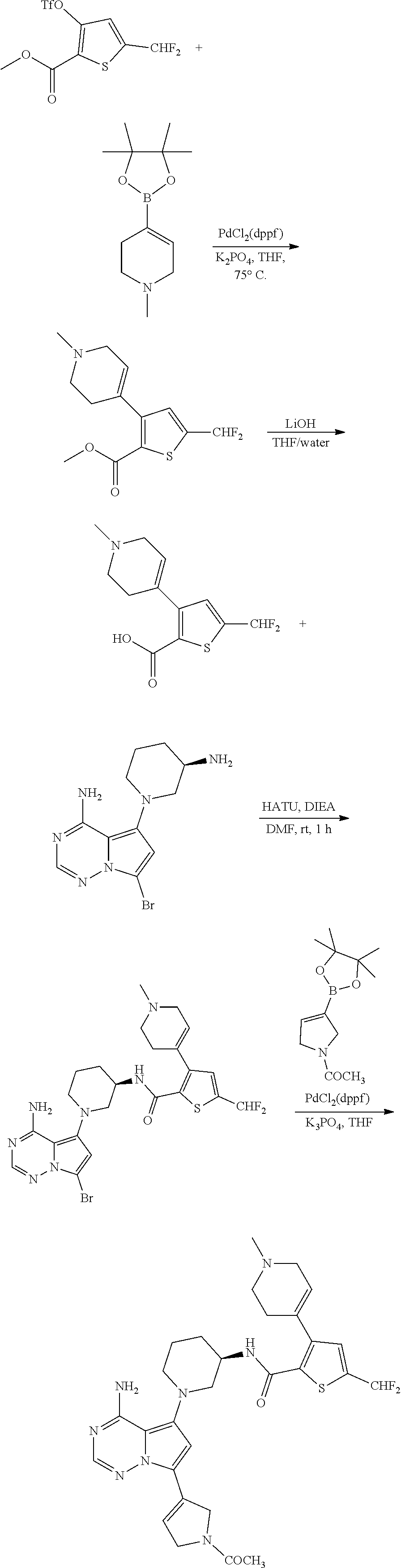

### Example 322A. Methyl 5-(difluoromethyl)-3-(1-methyl-1,2,3,6-tetrahydropyridin-4-yl)thiophene-2-carboxylate

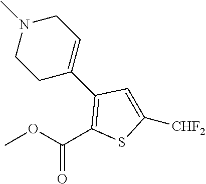 [SMILES: COC(=O)c1sc(C(F)F)cc1C1=CCN(C)CC1]

PdCl₂(dppf) (108 mg 0.147 mmol) was added to a solution of methyl 5-(difluoromethyl)-3 (((trifluoromethyl)sulfonyl)oxy)thiophene-2-carboxylate (500 mg, 1.47 mmol), 1-methyl-4-(4,4,5,5-tetramethyl-1,3,2-dioxaborolan-2-yl)-1,2,3,6-tetrahydropyridine (492 mg, 2.20 mmol) and tripotassium phosphate (2.20 mL 2M aq. solution, 4.41 mmol) in THF (10 mL). The reaction mixture was heated at 75° C. for 6 h, cooled and partitioned between water (50 mL) and ethyl acetate (50 mL). The organic layer was separated, dried over sodium sulphate and concentrated under reduced pressure. The resultant residue was purified by flash chromatography (10% DCM/MeOH) to afford Example 322A (300 mg, 70% yield) as a brown semisolid. MS: [M+H]⁺=288.2.

### Example 322B. 5-(difluoromethyl)-3-(1-methyl-1,2,3,6-tetrahydropyridin-4-yl)thiophene-2-carboxylic acid

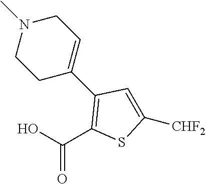 [SMILES: CN1CC=C(c2cc(C(F)F)sc2C(=O)O)CC1]

Analogous to the procedure for the preparation of Example 314C, reaction of methyl 5-(difluoromethyl)-3-(1-methyl-1,2,3,6-tetrahydropyridin-4-yl)thiophene-2-carboxylate (300 mg, 1.044 mmol) and LiOH (50.0 mg, 2.09 mmol) afforded Example 322B (270 mg, 95% yield) as a yellow solid. MS: [M+H]⁺=274.2.

### Example 322C. (R)—N-(1-(4-amino-7-bromopyrrolo[2,1-f][1,2,4]triazin-5-yl)piperidin-3-yl)-5-(difluoromethyl)-3-(1-methyl-1,2,3,6-tetrahydropyridin-4-yl)thiophene-2-carboxamide

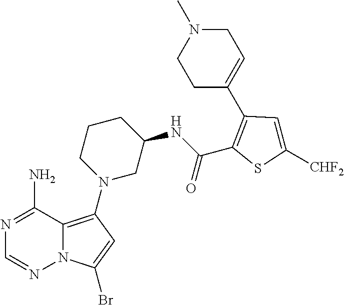 [SMILES: CN1CC=C(c2cc(C(F)F)sc2C(=O)N[C@@H]2CCCN(c3cc(Br)n4ncnc(N)c34)C2)CC1]

Analogous to the procedure for the preparation of Example 314D, reaction of 5-(difluoromethyl)-3-(1-methyl-1,2,3,6-tetrahydropyridin-4-yl)thiophene-2-carboxylic acid (176 mg, 0.643 mmol) and (R)-5-(3-aminopiperidin-1-yl)-7-bromopyrrolo[2,1-f][1,2,4]triazin-4-amine (200 mg, 0.643 mmol) afforded Example 322C (250 mg, 69% yield) as a yellow solid. MS: [M+H]⁺=568.3.

### Examples 322

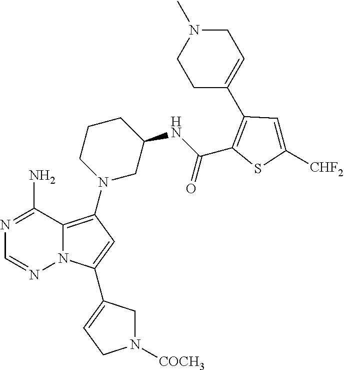 [SMILES: CC(=O)N1CC=C(c2cc(N3CCC[C@@H](NC(=O)c4sc(C(F)F)cc4C4=CCN(C)CC4)C3)c3c(N)ncnn23)C1]

PdCl₂(dppf)-DCM adduct (7.21 mg, 8.83 μmol) was added to a solution Example 322C (50 mg, 0.088 mmol), 1-(3-(4,4,5,5-tetramethyl-1,3,2-dioxaborolan-2-yl)-2,5-dihydro-1H-pyrrol-1-yl)ethan-1-one (31.4 mg, 0.132 mmol) and tripotassium phosphate (0.132 mL 2M solution, 0.265 mmol) in THF (2 mL). The reaction mixture was heated at 75° C. for 4 h, cooled and filtered. The filtrate was concentrated and purified by preparative HPLC to afford Example 322 (4.4 mg, 8% yield) LC-MS Method E: RT=1.30 min, [M+H]⁺=597.2; LC-MS Method F: RT=0.95 min, [M+H]⁺=597.2; ¹H-NMR (400 MHz, DMSO-d₆) δ ppm 8.90 (s, 1H), 8.33 (br s, 1H), 7.87 (d, J=2.0 Hz, 1H), 7.46-7.10 (m, 2H), 6.91-6.73 (m, 2H), 6.67 (d, J=10.0 Hz, 1H), 5.85 (br d, J=2.2 Hz, 1H), 4.74-4.67 (m, 1H), 4.54-4.45 (m, 3H), 4.29-4.24 (m, 1H), 4.19-4.06 (m, 1H), 3.14-3.09 (m, 1H), 2.93-2.69 (m, 5H), 2.38 (br s, 4H), 2.16-1.98 (m, 6H), 1.85 (s, 2H), 1.62-1.41 (m, 1H).

### Example 323

### Preparation of (R)—N-(1-(4-amino-7-(4-carbamoyl-3-fluorophenyl)pyrrolo[2,1-f][1,2,4]triazin-5-yl)piperidin-3-yl)-5-(difluoromethyl)-3-(1-yl)-5-(difluoromethyl)-3-(1-methyl-1,2,3,6-tetrahydropyridin-4-yl)thiophene-2-carboxamide

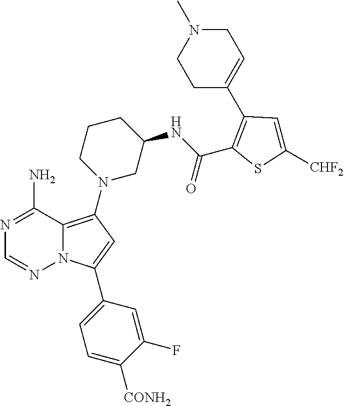 [SMILES: CN1CC=C(c2cc(C(F)F)sc2C(=O)N[C@@H]2CCCN(c3cc(-c4ccc(C(N)=O)c(F)c4)n4ncnc(N)c34)C2)CC1]

Analogous to the procedure for the preparation of Example 322, reaction of ((R)—N-(1-(4-amino-7-bromopyrrolo[2,1-f][1,2,4]triazin-5-yl)piperidin-3-yl)-5-(difluoromethyl)-3-(1-methyl-1,2,3,6-tetrahydropyridin-4-yl)thiophene-2-carboxamide (50 mg, 0.088 mmol), 2-fluoro-4-(4,4,5,5-tetramethyl-1,3,2-dioxaborolan-2-yl)benzamide (35.1 mg, 0.132 mmol) afforded Example 323 (8.2 mg, 14% yield). LC-MS Method E: RT=1.36 min, [M+H]⁺=625.2; LC-MS Method F: RT=0.97 min, [M+H]⁺=625.2; ¹H-NMR (400 MHz, DMSO-d₆) δ ppm 8.38 (br s, 1H), 8.13 (dd, J=1.5, 13.0 Hz, 1H), 8.07-7.98 (m, 2H), 7.92 (s, 1H), 7.74 (t, J=8.2 Hz, 1H), 7.65 (br dd, J=1.2, 13.0 Hz, 2H), 7.47-7.39 (m, 1H), 7.30-7.07 (m, 2H), 6.93 (br s, 1H), 5.88-5.77 (m, 1H), 4.24-4.15 (m, 1H), 3.20-3.16 (m, 1H), 2.99-2.63 (m, 6H), 2.38 (br d, J=0.7 Hz, 3H), 2.17-1.92 (m, 3H), 1.81 (s, 3H), 1.62-1.41 (m, 1H).

### Example 324

### Preparation of methyl (R)-3-(4-amino-5-(3-(3-(2-aminoethoxy)-5-(difluoromethyl)thiophene-2-carboxamido)piperidin-1-yl)pyrrolo[2,1-f][1,2,4]triazin-7-yl)-2,5-dihydro-1H-pyrrole-1-carboxylate

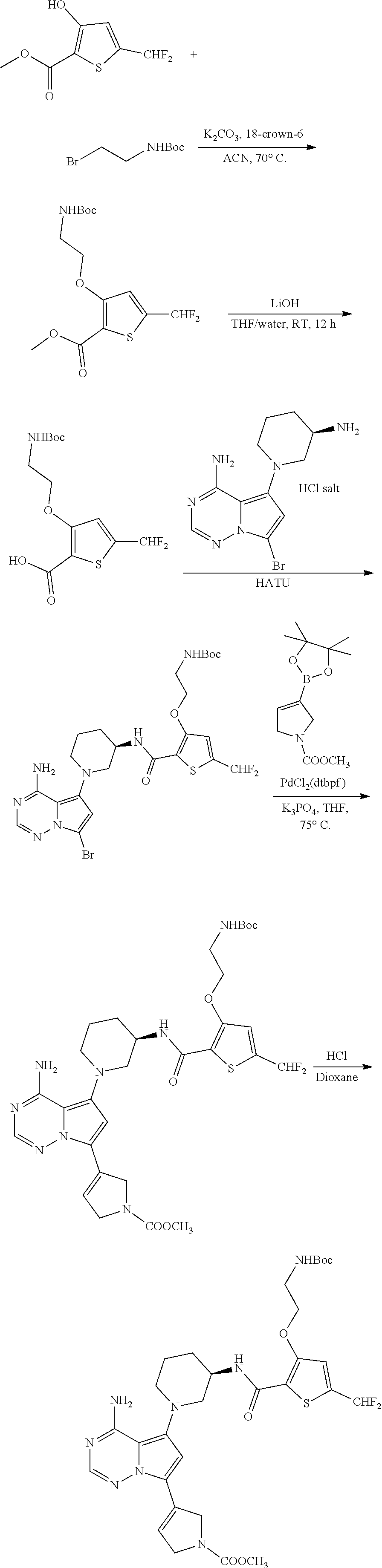

### Example 324A. Methyl 3-(2-((tert-butoxycarbonyl)amino)ethoxy)-5-(difluoromethyl)thiophene-2-carboxylate

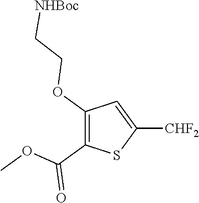 [SMILES: COC(=O)c1sc(C(F)F)cc1OCCNC(=O)OC(C)(C)C]

K₂CO₃ (996 mg, 7.21 mmol) was added to a solution of methyl 5-(difluoromethyl)-3-hydroxythiophene-2-carboxylate (500 mg, 2.402 mmol), tert-butyl (2-bromoethyl)carbamate (807 mg, 3.60 mmol) and 18-crown-6 (63 mg, 0.24 mmol) in acetonitrile (14 mL). It was stirred at 70° C. for 6 h, cooled and partitioned between water (50 mL) and ethyl acetate (50 mL). Organic layer was separated, dried over sodium sulphate and concentrated under reduced pressure to afford Example 324A (700 mg, 83% yield) as a yellow oil. MS: [M+Na]⁺=374.3.

### Example 324B. 3-(2-((tert-butoxycarbonyl)amino)ethoxy)-5-(difluoromethyl)thiophene-2-carboxylic acid. Li-salt

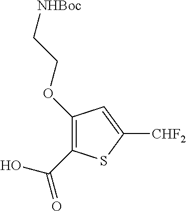 [SMILES: CC(C)(C)OC(=O)NCCOc1cc(C(F)F)sc1C(=O)O]

Analogous to the procedure for the preparation of Example 314C, reaction of methyl 3-(2-((tert-butoxycarbonyl)amino)ethoxy)-5-chlorothiophene-2-carboxylate) (1.5 g, 4.47 mmol) and LiOH (0.214 g, 8.93 mmol) afforded Example 324B (1300 mg, 90% yield) as a yellow solid. MS: [M+Na]⁺=360.3.

### Example 324C. tert-butyl (R)-(2-((2-((1-(4-amino-7-bromopyrrolo[2,1-f][1,2,4]triazin-5-yl)piperidin-3-yl)carbamoyl)-5-(difluoromethyl)thiophen-3-yl)oxy)ethyl)carbamate

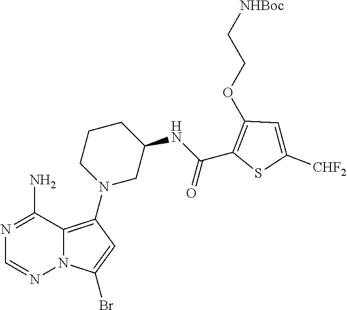 [SMILES: CC(C)(C)OC(=O)NCCOc1cc(C(F)F)sc1C(=O)N[C@@H]1CCCN(c2cc(Br)n3ncnc(N)c23)C1]

Analogous to the procedure for the preparation of Example 314D, reaction of Example 324B (434 mg, 1.285 mmol) and (R)-5-(3-aminopiperidin-1-yl)-7-bromopyrrolo[2,1-f][1,2,4]triazin-4-amine (400 mg, 1.285 mmol) afforded Example 324C (500 mg, 62% yield) as a yellow solid. MS: [M+H]⁺=632.4.

### Examples 324D. Methyl (R)-3-(4-amino-5-(3-(3-(2-((tert-butoxycarbonyl)amino)ethoxy)-5-(difluoromethyl)thiophene-2-carboxamido)piperidin-1-yl)pyrrolo[2,1-f][1,2,4]triazin-7-yl)-2,5-dihydro-1H-pyrrole-1-carboxylate

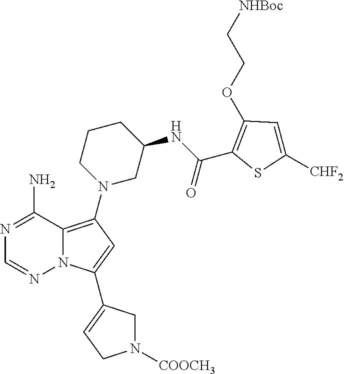 [SMILES: COC(=O)N1CC=C(c2cc(N3CCC[C@@H](NC(=O)c4sc(C(F)F)cc4OCCNC(=O)OC(C)(C)C)C3)c3c(N)ncnn23)C1]

[1,1′-Bis(di-tert-butylphosphino)ferrocene]dichloropalladium(II)] (10.34 mg, 0.016 mmol) was added to a solution of Example 324C (100 mg, 0.159 mmol), methyl 3-(4,4,5,5-tetramethyl-1,3,2-dioxaborolan-2-yl)-2,5-dihydro-1H-pyrrole-1-carboxylate (60.2 mg, 0.238 mmol) and tripotassium phosphate (0.159 mL 2M solution, 0.317 mmol) in THF (3 mL). The reaction mixture was heated at 70° C. for 4 h, cooled and partitioned between water (50 mL) and ethyl acetate (50 mL). The organic layer was separated, dried over sodium sulphate and concentrated under reduced pressure. The resultant residue was purified by flash chromatography (7% DCM/MeOH) to afford Example 324D (80 mg, 70% yield) as a yellow solid. MS: [M+H]⁺=677.5.

### Examples 324

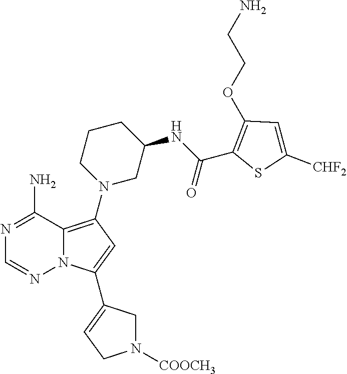 [SMILES: COC(=O)N1CC=C(c2cc(N3CCC[C@@H](NC(=O)c4sc(C(F)F)cc4OCCN)C3)c3c(N)ncnn23)C1]

A suspension of Example 324D (70 mg, 0.103 mmol) in HCl (1 mL, 4N) in dioxane was stirred at rt for 4 h. The reaction mixture was evaporated to dryness and purified by preparative HPLC to afford Examples 324 (15.6 mg, 25% yield). LC-MS Method E: RT=1.39 min, [M+H]⁺=577.2; LC-MS Method F: RT=1.06 min, [M+H]⁺=577.2; ¹H-NMR (400 MHz, DMSO-d₆) δ ppm 7.98-7.90 (m, 2H), 7.86 (s, 1H), 7.53 (s, 1H), 7.26 (t, J=55.0 Hz, 1H), 6.94 (bs, 1H), 6.82-6.68 (m, 2H), 4.60-4.49 (m, 2H), 4.35-4.24 (m, 4H), 4.20 (br d, J=5.1 Hz, 1H), 3.66 (d, J=4.4 Hz, 3H), 3.19-3.10 (m, 1H), 3.02-2.93 (m, 3H), 2.85-2.69 (m, 2H), 1.91-1.75 (m, 3H), 1.63-1.50 (m, 1H).

The following Examples in Table 19 were prepared using the same procedure as shown in Example 324. Example 324C was coupled with the appropriate boronic acid or boronate ester, using potassium phosphate tribasic monohydrate (2M aq. soln.) and [1,1′-bis(di-tert-butylphosphino)ferrocene]dichloropalladium(II)] in THF at 75° C. for 6 h. Then, deprotection of Boc was done using 4N HCl in dioxane to afford Example 325 and 326.

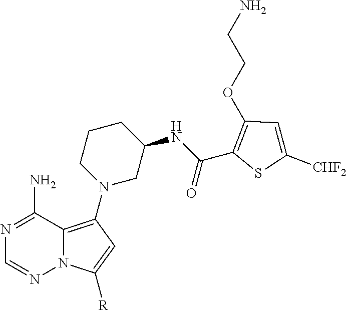

TABLE 19

| | | | | HPLC | |
|---|---|---|---|---|---|
| | Structure | | LCMS | Method, | |
| Example | (R) | Name | (M + H)⁺ | RT (min.) | ¹H NMR |
| 325 | 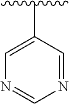 [SMILES: *c1cncnc1] | (R)-N-(1-(4-amino-7-(pyrimidin- 5-yl)pyrrolo[2,1-f][1,2,4]triazin- 5-yl)piperidin-3-yl)-3-(2- aminoethoxy)-5- (difluoromethyl)thiophene-2- carboxamide | 530.20 | E: 1.14 F: 0.89 | (400 MHz, DMSO-d₆) δ ppm 9.48 (s, 2H), 9.10 (s, 1H), 8.16 (s, 1H), 7.93 (s, 1H), 7.79-7.73 (m, 1H), 7.55 (s, 1H), 7.31 (s, 1H), 7.20 (t, J = 72.6 Hz, 1H), 6.92 (bs, 1H), 4.33 (br s, 2H), 4.24-4.19 (m, 1H), 3.10 (br d,) = 4.2 Hz, 4H), 2.90-2.76 (m, 3H), 1.98-1.77 (m, 4H), 1.66-1.54 (m, 1H) |
| 326 | 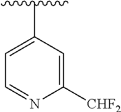 [SMILES: *c1ccnc(C(F)F)c1] | (R)-N-(1-(4-amino-7-(2- (difluoromethyl)pyridin-4- yl)pyrrolo[2,1-f][1,2,4]triazin-5- yl)piperidin-3-yl)-3-(2- aminoethoxy)-5- (difluoromethyl)thiophene-2- carboxamide | 579.20 | E: 1.45 F: 1.07 | (400 MHz, DMSO-d₆) δ ppm 8.70 (d, J = 5.3 Hz, 1H), 8.47 (s, 1H), 8.29 (dd, J = 1.8, 4.5 Hz, 1H), 8.25-8.15 (m, 1H), 8.05-7.92 (m, 3H), 7.54 (s, 1H), 7.47-7.36 (m, 1H), 7.28-7.18 (m, 1H), 7.14-7.06 (m, 1H), 6.97 (d, J = 4.5 Hz, 2H), 4.45-4.40 (m, 2H), 4.22-4.16 (m, 1H), 3.28-3.23 (m, 3H), 3.09-3.02 (m, 1H), 2.86-2.75 (m, 1H), 1.99-1.93 (m, 1H), 1.88-1.80 (m, 3H), 1,65-1.57 (m, 1H) |

### Example 327

### Preparation of (R)—N-(1-(4-amino-7-(pyrimidin-5-yl)pyrrolo[2,1-f][1,2,4]triazin-5-yl)piperidin-3-yl)-3-(2-aminoethoxy)-5-chlorothiophene-2-carboxamide

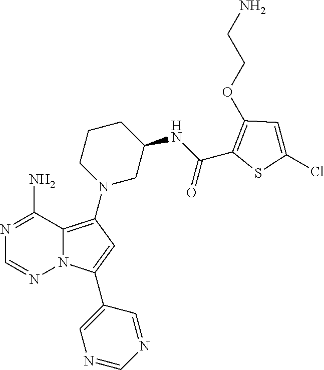 [SMILES: NCCOc1cc(Cl)sc1C(=O)N[C@@H]1CCCN(c2cc(-c3cncnc3)n3ncnc(N)c23)C1]

Analogous to the procedure for the preparation of Example 325, using methyl 5-chloro-3-hydroxythiophene-2-carboxylate afforded Example 327 (5.6 mg, 8% yield). LC-MS Method E: RT=1.137 min, [M+H]⁺=514.1; LC-MS Method F: RT=0.892 min, [M+H]⁺=514.1; ¹H NMR (400 MHz, DMSO-d₆) δ ppm 9.53-9.35 (m, 2H), 9.15-8.96 (m, 1H), 8.01-7.81 (m, 2H), 7.68-7.50 (m, 1H), 7.38-7.21 (m, 2H), 6.93 (bs, 1H), 4.32-4.25 (m, 2H), 4.21-4.11 (m, 1H), 3.14-2.93 (m, 5H), 2.90-2.73 (m, 3H), 2.02-1.77 (m, 3H), 1.69-1.49 (m, 1H).

### Example 328

### Preparation of (R)-3-(2-acetamidoethoxy)-N-(1-(7-(1-acetyl-2,5-dihydro-1H-pyrrol-3-yl)-4-aminopyrrolo[2,1-f][1,2,4]triazin-5-yl)piperidin-3-yl)-5-chlorothiophene-2-carboxamide

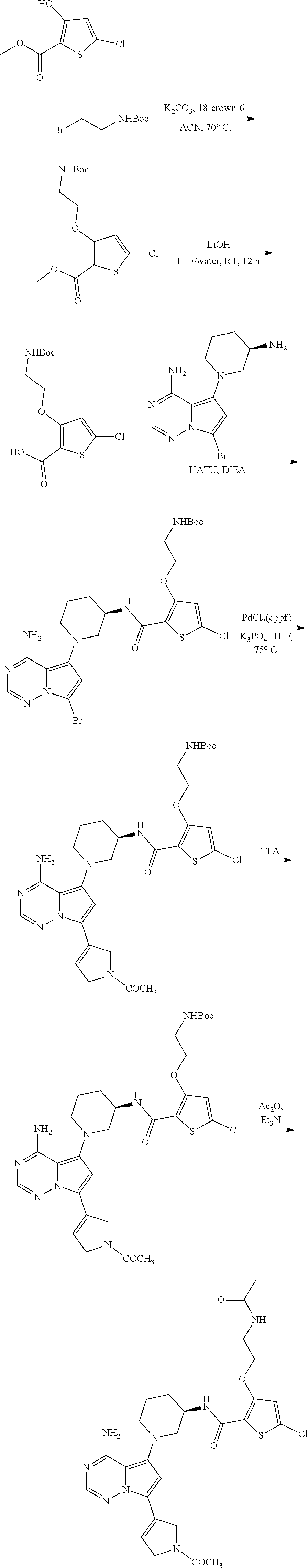

### Example 328A. Methyl 3-(2-((tert-butoxycarbonyl)amino)ethoxy)-5-chlorothiophene-2-carboxylate

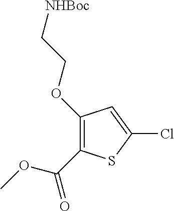 [SMILES: COC(=O)c1sc(Cl)cc1OCCNC(=O)OC(C)(C)C]

Analogous to the procedure for the preparation of Example 324A, reaction of methyl 5-chloro-3-hydroxythiophene-2-carboxylate (1 g, 5.19 mmol) and tert-butyl (2-bromoethyl)carbamate (1.745 g, 7.79 mmol) afforded Example 328A (1300 mg, 75% yield) as a yellow oil. MS: [M+Na]=358.2.

### Example 328B. 3-(2-((tert-butoxycarbonyl)amino)ethoxy)-5-chlorothiophene-2-carboxylic acid, Li-salt

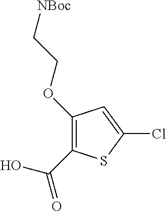

Analogous to the procedure for the preparation of Example 314C, reaction of methyl 3-(2-((tert-butoxycarbonyl)amino)ethoxy)-5-chlorothiophene-2-carboxylate (1.5 g, 4.47 mmol) and LiOH (0.214 g, 8.93 mmol) afforded Example 328B (1300 mg, 90% yield) as an off-white solid. MS: [M+Na]⁺=346.2.

### Example 328C. tert-butyl (R)-(2-((2-((1-(4-amino-7-bromopyrrolo[2,1-f][1,2,4]triazin-5-yl)piperidin-3-yl)carbamoyl)-5-chlorothiophen-3-yl)oxy)ethyl)carbamate

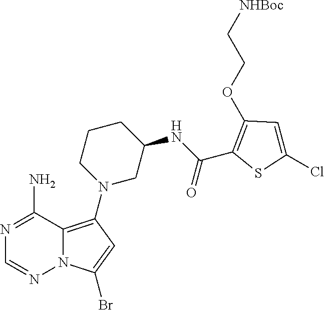 [SMILES: CC(C)(C)OC(=O)NCCOc1cc(Cl)sc1C(=O)N[C@@H]1CCCN(c2cc(Br)n3ncnc(N)c23)C1]

Analogous to the procedure for the preparation of Example 314D, reaction of 3-(2-((tert-butoxycarbonyl)amino)ethoxy)-5-chlorothiophene-2-carboxylic acid (993 mg, 3.08 mmol) and (R)-5-(3-aminopiperidin-1-yl)-7-bromopyrrolo[2,1-f][1,2,4]triazin-4-amine (800 mg, 2.57 mmol) afforded Example 328C (600 mg, 38% yield) as a yellow solid. MS: [M+H]⁺=616.3.

### Example 328D. tert-butyl (R)-(2-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)ethyl)carbamate

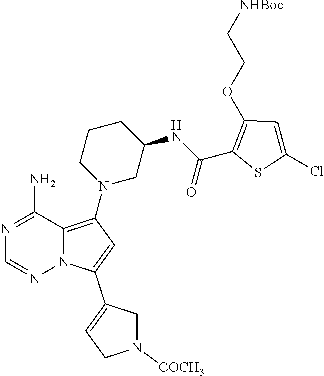 [SMILES: CC(=O)N1CC=C(c2cc(N3CCC[C@@H](NC(=O)c4sc(Cl)cc4OCCNC(=O)OC(C)(C)C)C3)c3c(N)ncnn23)C1]

PdCl₂(dppf)-DCM adduct (80 mg, 0.098 mmol) was added to the suspension of Example 328C (600 mg, 0.976 mmol), 1-(3-(4,4,5,5-tetramethyl-1,3,2-dioxaborolan-2-yl)-2,5-dihydro-1H-pyrrol-1-yl)ethan-1-one (347 mg, 1.46 mmol) and tripotassium phosphate (0.976 mL 2M aq. solution, 1.95 mmol) in THF (10 mL). The reaction mixture was heated at 70° C. for 4 h, cooled to rt and partitioned between water (50 mL) and ethyl acetate (50 mL). Organic layer was separated, dried over sodium sulphate and concentrated under reduced pressure. The resultant residue was purified by flash chromatography (7% DCM/MeOH) which afforded Example 328D (400 mg, 64% yield) as a yellow solid. MS: [M+H]⁺=645.4.

### Example 328E. (R)—N-(1-(7-(1-acetyl-2,5-dihydro-1H-pyrrol-3-yl)-4-aminopyrrolo[2,1-f][1,2,4]triazin-5-yl)piperidin-3-yl)-3-(2-aminoethoxy)-5-chlorothiophene-2-carboxamide

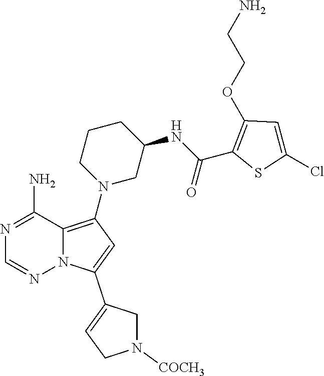 [SMILES: CC(=O)N1CC=C(c2cc(N3CCC[C@@H](NC(=O)c4sc(Cl)cc4OCCN)C3)c3c(N)ncnn23)C1]

TFA (0.955 mL, 12.40 mmol) was added to a solution of Example 328D (400 mg, 0.620 mmol) in DCM (8 mL) at rt and stirred at it for 2 h followed by evaporation to dryness to afforded Example 328E (300 mg, 89% yield) as a brown oil. MS: [M+H]⁺=545.2.

### Example 328

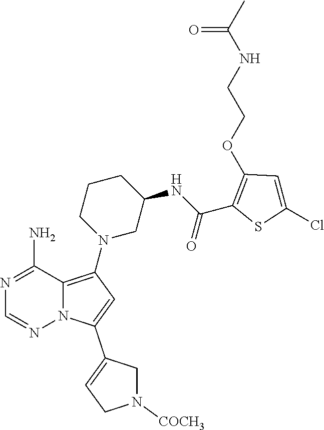 [SMILES: CC(=O)NCCOc1cc(Cl)sc1C(=O)N[C@@H]1CCCN(c2cc(C3=CCN(C(C)=O)C3)n3ncnc(N)c23)C1]

Acetic anhydride (8.66 μl, 0.092 mmol) in DCM (0.5 mL) was added dropwise to a solution of Example 328E (50 mg, 0.092 mmol) and triethylamine (0.038 mL, 0.275 mmol) in DCM (2 mL). The mixture was stirred at rt for 1 h and partitioned between water (10 mL) and DCM (10 mL). The organic layer was separated, dried over sodium sulphate and concentrated under reduced pressure. Residue was purified by HPLC to afford Example 328 (7.6 mg, 13% yield). LC-MS Method E: RT=1.352 min, [M+H]⁺=587.2; LC-MS Method F: RT=1.092 min, [M+H]⁺=587.2; ¹H NMR (400 MHz, DMSO-d₆) δ ppm 8.18-8.10 (m, 1H), 7.97-7.97 (m, 1H), 8.06-7.91 (m, 1H), 7.89-7.83 (m, 1H), 7.32 (s, 2H), 6.86-6.74 (m, 1H), 6.71 (d, J=13.2 Hz, 1H), 4.68 (br s, 1H), 4.55-4.43 (m, 2H), 4.31-4.10 (m, 4H), 3.45 (br dd, J=5.1, 10.0 Hz, 2H), 3.13-3.06 (m, 1H), 3.02-2.96 (m, 1H), 2.86-2.68 (m, 2H), 2.03 (d, J=16.1 Hz, 3H), 1.91-1.73 (m, 6H), 1.71-1.52 (m, 1H).

### Example 329

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-(2-(2,2-difluoroacetamido)ethoxy)thiophene-2-carboxamide

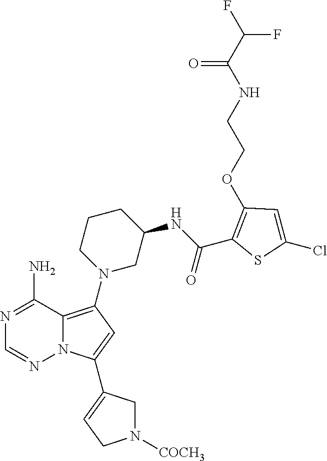 [SMILES: CC(=O)N1CC=C(c2cc(N3CCC[C@@H](NC(=O)c4sc(Cl)cc4OCCNC(=O)C(F)F)C3)c3c(N)ncnn23)C1]

Analogous to the procedure for the preparation of Example 328, reaction of Example 328D (50 mg, 0.092 mmol) and difluoroacetic anhydride (0.011 mL, 0.092 mmol) afforded Example 329 (13.7 mg, 24% yield). LC-MS Method E: RT=1.483 min, [M+H]⁺=624.2; LC-MS Method F: RT=1.233 min, [M+H]⁺=624.2; ¹H NMR (400 MHz, DMSO-d₆) δ ppm 9.11-9.02 (m, 1H), 8.04-7.94 (m, 1H), 7.87 (d, J=1.5 Hz, 1H), 7.33 (s, 1H), 7.21 (br dd, J=2.7, 6.6 Hz, 1H), 6.91 (bs, 1H), 6.89-6.74 (m, 1H), 6.68 (d, J=10.3 Hz, 1H), 6.44-6.11 (m, 1H), 4.69 (br s, 1H), 4.53-4.44 (m, 2H), 4.34-4.22 (m, 3H), 4.15 (br dd, J=4.8, 9.9 Hz, 1H), 3.64-3.52 (m, 2H), 3.14-3.07 (m, 1H), 3.02-2.94 (m, 1H), 2.82-2.75 (m, 1H), 2.68 (br d, J=1.7 Hz, 1H), 2.03 (d, J=15.4 Hz, 3H), 1.92-1.74 (m, 3H), 1.68-1.55 (m, 1H).

### Example 330

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-3-((S)-2-aminopropoxy)-5-chlorothiophene-2-carboxamide

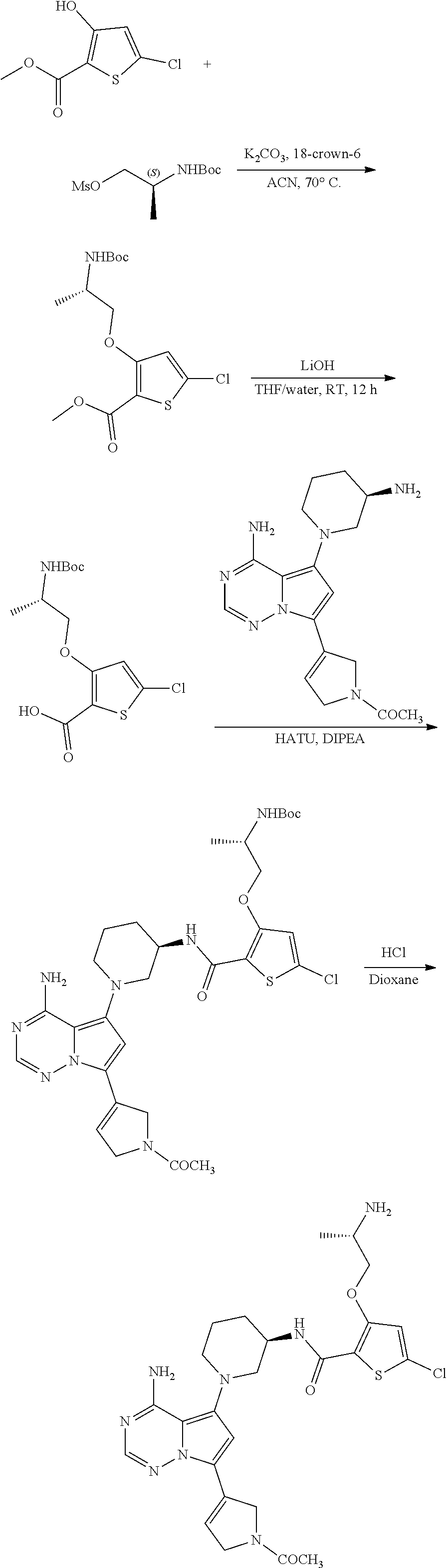

### Example 330A. Methyl (S)-3-(2-((tert-butoxycarbonyl)amino)propoxy)-5-chlorothiophene-2-carboxylate

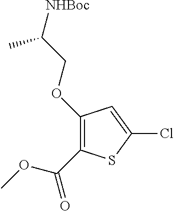 [SMILES: COC(=O)c1sc(Cl)cc1OC[C@H](C)NC(=O)OC(C)(C)C]

Analogous to the procedure for the preparation of Example 324A, reaction of (S)-2-((tert-butoxycarbonyl)amino)propyl methanesulfonate (526 mg, 2.077 mmol) and methyl 5-chloro-3-hydroxythiophene-2-carboxylate (250 mg, 1.30 mmol) afforded Example 330A (200 mg, 44% yield) as a yellow semisolid. MS: [M+Na]⁺=372.2.

### Example 330B. (S)-3-(2-((tert-butoxycarbonyl)amino)propoxy)-5-chlorothiophene-2-carboxylic acid

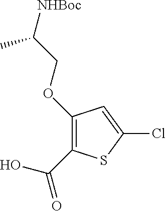 [SMILES: C[C@@H](COc1cc(Cl)sc1C(=O)O)NC(=O)OC(C)(C)C]

Analogous to the procedure for the preparation of Example 324C, reaction of methyl (S)-3-(2-((tert-butoxycarbonyl)amino)propoxy)-5-chlorothiophene-2-carboxylate (200 mg, 0.572 mmol) and LiOH (27.4 mg, 1.143 mmol) afforded Example 330B (170 mg, 89% yield) as an off-white solid. MS: [M−Boc+2H]⁺=236.1.

### Example 330C. tert-butyl ((S)-1-((2-(((R)-1-(4-amino-7-bromopyrrolo[2,1-f][1,2,4]triazin-5-yl)piperidin-3-yl)carbamoyl)-5-chlorothiophen-3-yl)oxy)propan-2-yl)carbamate

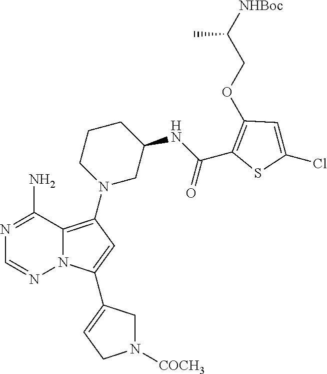 [SMILES: CC(=O)N1CC=C(c2cc(N3CCC[C@@H](NC(=O)c4sc(Cl)cc4OC[C@H](C)NC(=O)OC(C)(C)C)C3)c3c(N)ncnn23)C1]

Analogous to the procedure for the preparation of Example 324D, reaction of(S)-3-(2-((tert-butoxycarbonyl)amino)propoxy)-5-chlorothiophene-2-carboxylic acid (90 mg, 0.267 mmol) and ((R)-1-(3-(4-amino-5-(3-aminopiperidin-1-yl)pyrrolo[2,1-f][1,2,4]triazin-7-yl)-2,5-dihydro-1H-pyrrol-1-yl)ethan-1-one (70 mg, 0.205 mmol) afforded Example 330C (90 mg, 67% yield) as a dark red gummy solid. MS: [M+H]⁺=659.5.

### Example 330

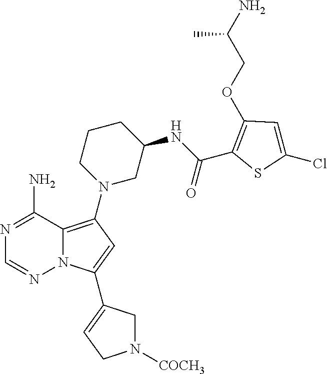 [SMILES: CC(=O)N1CC=C(c2cc(N3CCC[C@@H](NC(=O)c4sc(Cl)cc4OC[C@H](C)N)C3)c3c(N)ncnn23)C1]

A suspension of Example 330C (50 mg, 0.076 mmol) in 4N HCl in dioxane (758 μl, 3.03 mmol) was stirred at rt for 5 h. The reaction mixture was evaporated to dryness and purified by preparative HPLC to afford Example 330 (11.8 mg, 26% yield). LC-MS Method E: RT=1.269 min, [M+H]⁺=559.2; LC-MS Method F: RT=0.935 min, [M+H]⁺=559.3.

### Example 331

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-3-((1-aminocyclopropyl)methoxy)-5-chlorothiophene-2-carboxamide

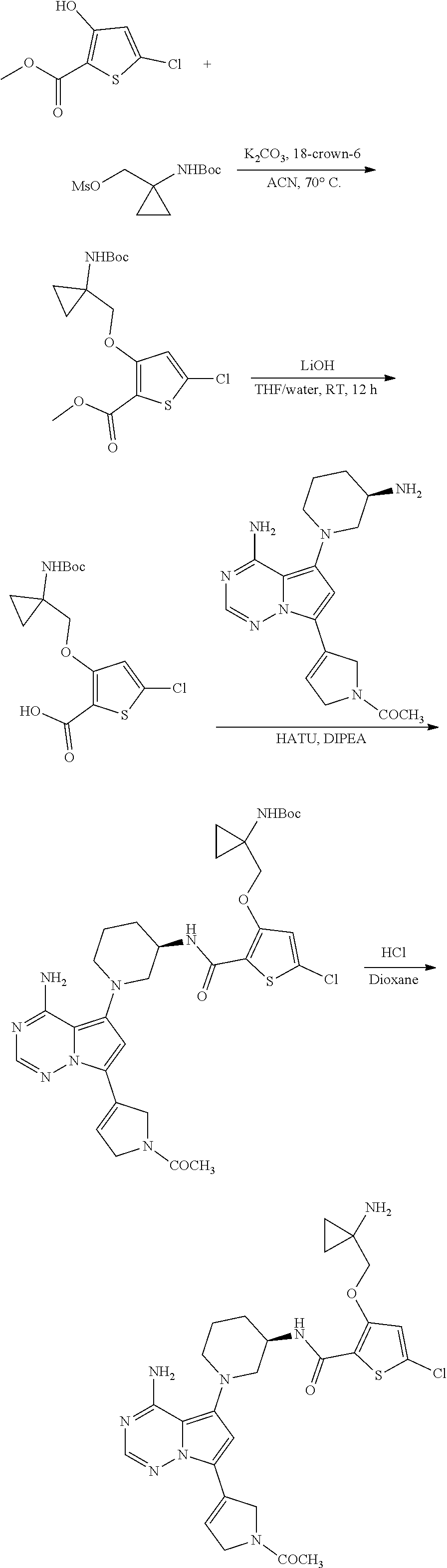

### Example 331A. Methyl 3-((1-((tert-butoxycarbonyl)amino)cyclopropyl)methoxy)-5-chlorothiophene-2-carboxylate

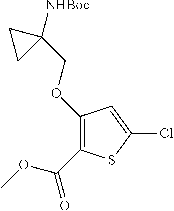 [SMILES: COC(=O)c1sc(Cl)cc1OCC1(NC(=O)OC(C)(C)C)CC1]

analogous to the procedure for the preparation fo the Example 324A, reaction of methyl 5-chloro-3-hydroxythiophene-2-carboxylate (250 mg, 1.298 mmol) and (1-((tert-butoxycarbonyl)amino)cyclopropyl)methyl methanesulfonate (55 mg, 2.077 mmol) afforded Example 331A (150 mg, 32% yield) as a yello gummy solid. MS: [M−Boc+2H]⁺=262.1.

### Example 331B. 3-((1-((tert-butoxycarbonyl)amino)cyclopropyl)methoxy)-5-chlorothiophene-2-carboxylic acid

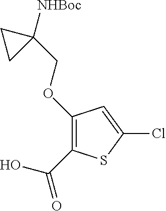 [SMILES: CC(C)(C)OC(=O)NC1(COc2cc(Cl)sc2C(=O)O)CC1]

Analogous to the procedure for the preparation of Example 314C, reaction of methyl 3-((1-((tert-butoxycarbonyl)amino)cyclopropyl)methoxy)-5-chlorothiophene-2-carboxylate (150 mg, 0.415 mmol) and LiOH (19.9 mg, 0.829 mmol) afforded Example 331B (130 mg, 90% yield) as an off-white solid. MS: [M+Na]⁺=370.3.

### Example 331C. tert-butyl (R)-(1-(((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)methyl)cyclopropyl)carbamate

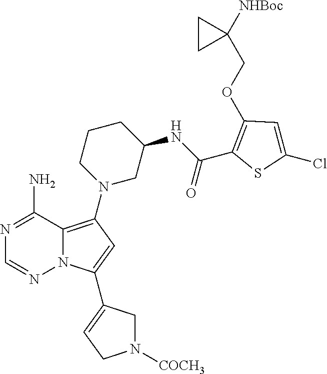 [SMILES: CC(=O)N1CC=C(c2cc(N3CCC[C@@H](NC(=O)c4sc(Cl)cc4OCC4(NC(=O)OC(C)(C)C)CC4)C3)c3c(N)ncnn23)C1]

Analogous to the procedure for the preparation of Example 314D, reaction of Example 331B (93 mg, 0.267 mmol) and (R)-1-(3-(4-amino-5-(3-aminopiperidin-1-yl)pyrrolo[2,1-f][1,2,4]triazin-7-yl)-2,5-dihydro-1H-pyrrol-1-yl)ethan-1-one (70 mg, 0.205 mmol) afforded Example 331C (90 mg, 65% yield) as a dark red gummy solid. MS: [M+H]⁺=671.5.

### Example 331

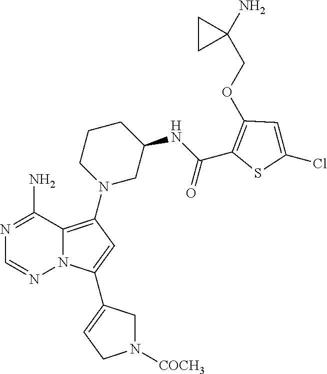 [SMILES: CC(=O)N1CC=C(c2cc(N3CCC[C@@H](NC(=O)c4sc(Cl)cc4OCC4(N)CC4)C3)c3c(N)ncnn23)C1]

Analogous to the procedure for the preparation of Example 330, reaction of Example 331C (40 mg, 0.060 mmol) and HCl (1 mL, 4N) afforded Example 331 (12.2 mg, 35% yield). LC-MS Method E: RT=1.450 min, [M+H]⁺=571.2; LC-MS Method F: RT=0.943 min, [M+H]⁺=571.3; ¹H NMR (400 MHz, DMSO-d₆) δ ppm 8.73-8.50 (m, 3H), 7.89 (s, 1H), 7.40-7.28 (m, 2H), 7.01 (s, 1H), 6.83-6.75 (m, 1H), 6.71 (d, J=8.1 Hz, 1H), 4.69 (br s, 1H), 4.56-4.44 (m, 3H), 4.42-4.24 (m, 1H), 4.17-4.06 (m, 1H), 3.24-3.17 (m, 1H), 3.07-2.98 (m, 1H), 2.83-2.69 (m, 2H), 2.07-1.91 (m, 5H), 1.89-1.78 (m, 2H), 1.63-1.47 (m, 1H), 1.11-1.00 (m, 4H).

### Example 332

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-(2-(N-methylacetamido)ethoxy)thiophene-2-carboxamide

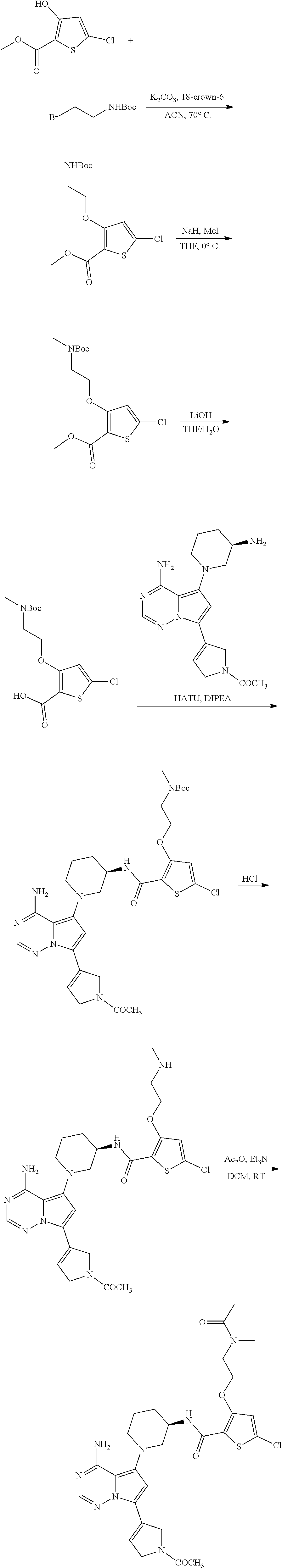

### Example 332A. Methyl 3-(2-((tert-butoxycarbonyl)(methyl)amino)ethoxy)-5-chlorothiophene-2-carboxylate

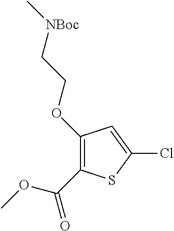 [SMILES: COC(=O)c1sc(Cl)cc1OCCN(C)C(=O)OC(C)(C)C]

NaH (0.268 g, 6.70 mmol) was added portionwise to a solution of methyl 3-(2-((tert-butoxycarbonyl)amino)ethoxy)-5-chlorothiophene-2-carboxylate (1.5 g, 4.47 mmol) and MeI (0.838 mL, 13.4 mmol) in THF (30 mL) kept at 0° C. The mixture was stirred at 0° C. for another 1 h. The reaction was quenched with ice water (10 mL) and diluted with ethyl acetate (50 mL). The organic layer was separated, dried over sodium sulphate and concentrated under reduced pressure. The resultant residue was purified by flash chromatography (10% DCM/MeOH) to afford Example 332A (520 mg, 33% yield) as a yellow semisolid. MS: [M−Boc+2H]⁺=250.2.

### Example 57B. 3-(2-((tert-butoxycarbonyl)(methyl)amino)ethoxy)-5-chlorothiophene-2-carboxylic acid

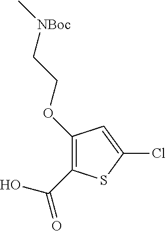 [SMILES: CN(CCOc1cc(Cl)sc1C(=O)O)C(=O)OC(C)(C)C]

Analogous to the procedure for the preparation of Example 314C, reaction of methyl 3-(2-((tert-butoxycarbonyl)(methyl)amino)ethoxy)-5-chlorothiophene-2-carboxylate (100 mg, 0.286 mmol) and LiOH (13.69 mg, 0.572 mmol) afforded Example 332B (80 mg, 83% yield) as a yellow solid. MS: [M−Boc+2H]⁺=236.2.

### Example 332C. tert-butyl (R)-(2-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)ethyl)(methyl)carbamate

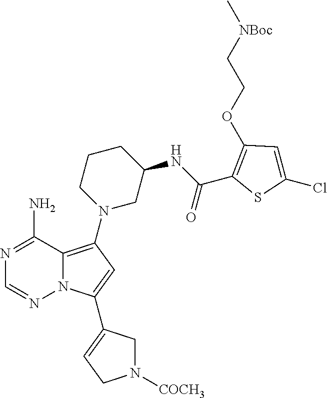 [SMILES: CC(=O)N1CC=C(c2cc(N3CCC[C@@H](NC(=O)c4sc(Cl)cc4OCCN(C)C(=O)OC(C)(C)C)C3)c3c(N)ncnn23)C1]

Analogous to the procedure for the preparation of Example 314D, reaction of Example 332B (68.8 mg, 0.205 mmol) and (R)-1-(3-(4-amino-5-(3-aminopiperidin-1-yl)pyrrolo[2,1-f][1,2,4]triazin-7-yl)-2,5-dihydro-1H-pyrrol-1-yl)ethan-1-one (70 mg, 0.205 mmol) afforded Example 332C (100 mg, 74% yield) as a yellow gummy solid. MS: [M+H]⁺=659.3.

### Example 332D

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-(2-(methylamino)ethoxy)thiophene-2-carboxamide

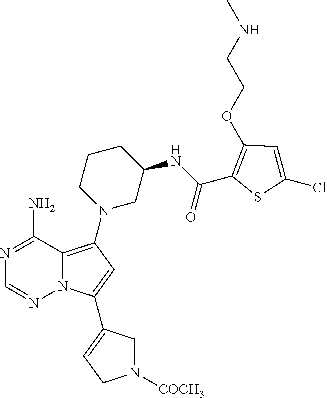 [SMILES: CNCCOc1cc(Cl)sc1C(=O)N[C@@H]1CCCN(c2cc(C3=CCN(C(C)=O)C3)n3ncnc(N)c23)C1]

A suspension of Example 332C (50 mg, 0.076 mmol) in 4N HCl in dioxane (379 μl, 1.517 mmol) was stirred at rt for 2 h. The reaction mixture was evaporated to dryness and purified by Preparative HPLC to afford Example 332D (7.5 mg, 17% yield). LC-MS Method E: RT=1.194 min, [M+H]⁺=559.2; LC-MS Method F: RT=0.911 min, [M+H]⁺=559.2; ¹H NMR (400 MHz, DMSO-d₆) δ ppm 8.74 (br s, 2H), 7.90 (s, 1H), 7.36 (s, 1H), 7.10 (s, 1H), 6.98 (s, 1H), 6.81-6.75 (m, 1H), 6.70 (d, J=9.3 Hz, 1H), 4.68 (br s, 1H), 4.70-4.65 (m, 1H), 4.51-4.42 (m, 5H), 4.28-4.24 (m, 1H), 4.13 (br s, 1H), 3.19-3.17 (m, 1H), 3.12-3.09 (m, 1H), 3.03-2.89 (m, 2H), 2.79-2.69 (m, 2H), 2.64 (br t, J=4.6 Hz, 3H), 2.03 (d, J=15.5 Hz, 3H), 1.82 (br s, 2H), 1.62-1.48 (m, 1H).

### Example 332

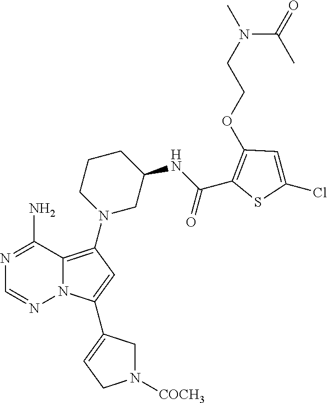 [SMILES: CC(=O)N(C)CCOc1cc(Cl)sc1C(=O)N[C@@H]1CCCN(c2cc(C3=CCN(C(C)=O)C3)n3ncnc(N)c23)C1]

Acetic anhydride (0.011 mL, 0.12 mmol) in DCM (0.5 mL) was added to a solution of Example 332D (60 mg, 0.107 mmol) and Et₃N (0.045 mL, 0.322 mmol) in DCM (2 mL). The mixture was stirred at rt for 1 h and partitioned between water (10 mL) and DCM (10 mL). Organic layer was separated, dried over sodium sulphate, concentrated and purified by Preparative HPLC to afford Example 332 (4.1 mg, 6% yield). LC-MS Method E: RT=1.389 min, [M+H]⁺=601.2; LC-MS Method F: RT=1.176 min, [M+H]⁺=601.3; ¹H NMR (400 MHz, DMSO-d₆) δ ppm 7.98 (br s, 1H), 7.86 (d, J=1.3 Hz, 1H), 7.28 (s, 2H), 6.88-6.73 (m, 2H), 6.69 (d, J=11.8 Hz, 1H), 4.68 (br d, J=2.3 Hz, 1H), 4.53-4.39 (m, 2H), 4.33-4.23 (m, 3H), 4.16 (br s, 1H), 3.80-3.60 (m, 2H), 3.14-3.07 (m, 1H), 3.00 (s, 3H), 2.82 (s, 3H), 2.06-1.96 (m, 6H), 1.90-1.68 (m, 4H).

### Example 333

### Methyl (R)-(2-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)ethyl)methyl)carbamate

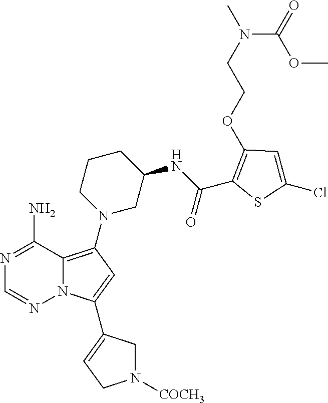 [SMILES: COC(=O)N(C)CCOc1cc(Cl)sc1C(=O)N[C@@H]1CCCN(c2cc(C3=CCN(C(C)=O)C3)n3ncnc(N)c23)C1]

Analogous to the procedure for the preparation of Example 332, reaction of Example 332D (60 mg, 0.107 mmol) and methyl carbonochloridate (9.1 μl, 0.12 mmol) afforded Example 333 (8 mg, 12% yield). LC-MS Method E: RT=1.617 min, [M+H]⁺=617.2.

### Example 334

### Methyl (R)-3-(4-amino-5-(3-(5-(difluoromethyl)-3-(2-(4-methylpiperazin-1-yl)ethoxy)thiophene-2-carboxamido)piperidin-1-yl)pyrrolo[2,1-f][1,2,4]triazin-7-yl)-2,5-dihydro-1H-pyrrole-1-carboxylate

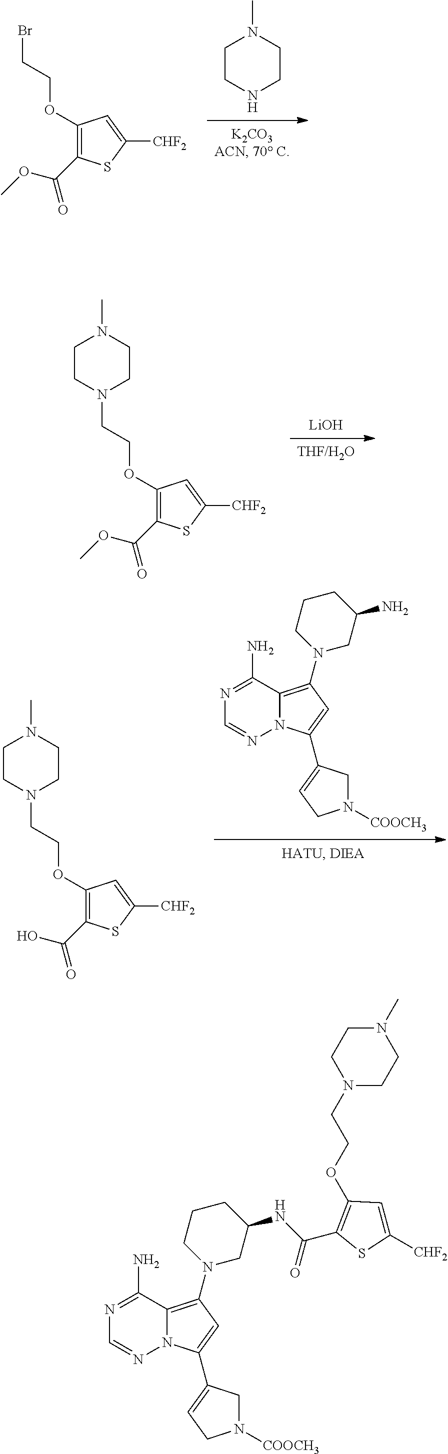

### Example 334A. Methyl 5-(difluoromethyl)-3-(2-(4-methylpiperazin-1-yl)ethoxy)thiophene-2-carboxylate

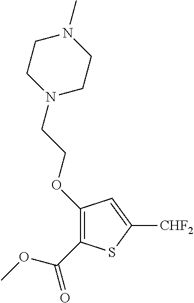 [SMILES: COC(=O)c1sc(C(F)F)cc1OCCN1CCN(C)CC1]

K₂CO₃ (197 mg, 1.43 mmol) was added to a solution of methyl 3-(2-bromoethoxy)-5-(difluoromethyl)thiophene-2-carboxylate (150 mg, 0.476 mmol) and 1-methylpiperazine (71.5 mg, 0.714 mmol) in acetonitrile (3 mL). It was stirred at 70° C. for 9 h, cooled to rt and partitioned between water (10 mL) and DCM (10 mL). Organic layer was separated, dried over sodium sulphate and concentrated under reduced pressure to afford Example 334A (140 mg, 88% yield) as a yellow oil. MS: [M+H]⁺=335.3.

### Example 334B. 5-(difluoromethyl)-3-(2-(4-methylpiperazin-1-yl)ethoxy)thiophene-2-carboxylic acid

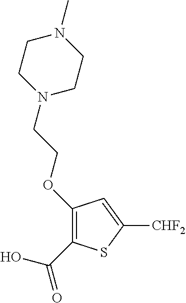 [SMILES: CN1CCN(CCOc2cc(C(F)F)sc2C(=O)O)CC1]

Analogous to the procedure for the preparation of Example 314C, reaction of Example 334A (120 mg, 0.359 mmol) and LiOH (17.2 mg, 0.718 mmol) afforded Example 334B (100 mg, 87% yield) as an off-white solid. MS: [M+H]⁺=321.3.

### Example 334

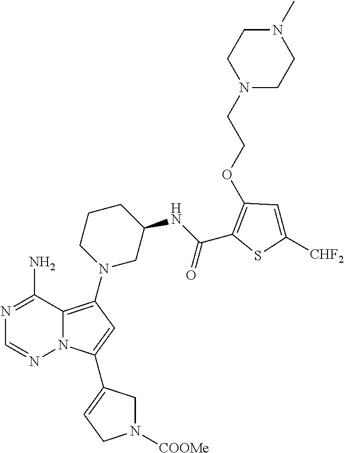 [SMILES: COC(=O)N1CC=C(c2cc(N3CCC[C@@H](NC(=O)c4sc(C(F)F)cc4OCCN4CCN(C)CC4)C3)c3c(N)ncnn23)C1]

DIEA (0.073 mL, 0.420 mmol) was added to a solution of Intermediate 31 (50 mg, 0.140 mmol), Example 334B (44.8 mg, 0.140 mmol) and HATU (53.2 mg, 0.140 mmol) in DMF (2 mL). The mixture was stirred at rt for 2 h and concentrated. The residue was purified by preparative HPLC to afford Example 334 (32.2 mg, 35% yield). LC-MS Method E: RT=1.452 min, [M+H]⁺=660.2; LC-MS Method F: RT=1.039 min, [M+H]⁺=660.3; ¹H NMR (400 MHz, DMSO-d₆) δ ppm 7.86 (s, 1H), 7.69-7.56 (m, 2H), 7.53 (d, J=1.5 Hz, 1H), 7.25 (t, J=57.2 Hz, 1H), 6.90 (bs, 1H), 6.80-6.64 (m, 2H), 4.60-4.48 (m, 2H), 4.43-4.25 (m, 4H), 4.25-4.11 (m, 1H), 3.66 (d, J=4.2 Hz, 3H), 3.18-3.12 (m, 1H), 2.97 (br s, 1H), 2.88-2.74 (m, 2H), 2.70-2.65 (m, 2H), 2.44-2.33 (m, 3H), 2.27-2.11 (m, 3H), 2.05 (s, 3H), 1.94-1.74 (m, 5H), 1.57 (br s, 1H).

### Example 335

### Preparation of (R)—N-(1-(4-amino-7-(2-(difluoromethyl)pyridin-4-yl)pyrrolo[2,1-f][1,2,4]triazin-5-yl)piperidin-3-yl)-5-(difluoromethyl)-3-(2-(4-methylpiperazin-1-yl)ethoxy)thiophene-2-carboxamide

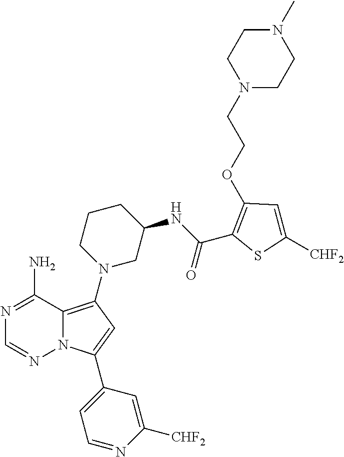 [SMILES: CN1CCN(CCOc2cc(C(F)F)sc2C(=O)N[C@@H]2CCCN(c3cc(-c4ccnc(C(F)F)c4)n4ncnc(N)c34)C2)CC1]

Analogous to the procedure for the preparation of Example 334, reaction of Example 334B (44.6 mg, 0.139 mmol) and Intermediate 27 afforded Example 335 (13.6 mg, 14% yield). LC-MS Method E: RT=1.588 min, [M+H]⁺=662.2; LC-MS Method F: RT=1.140 min, [M+H]⁺=662.2; ¹H NMR (400 MHz, DMSO-d₆) δ ppm 8.70 (d, J=5.5 Hz, 1H), 8.47 (s, 1H), 8.33-8.28 (m, 1H), 8.25-8.11 (m, 1H), 7.99 (s, 1H), 7.72-7.57 (m, 1H), 7.54 (s, 1H), 7.45 (s, 1H), 7.41-6.82 (m, 3H), 4.42-4.31 (m, 2H), 4.24 (br dd, J=2.5, 3.8 Hz, 1H), 3.23 (br s, 3H), 3.08-2.98 (m, 1H), 2.96-2.78 (m, 2H), 2.76-2.64 (m, 2H), 2.45-2.20 (m, 5H), 2.17-2.02 (m, 2H), 1.97-1.78 (m, 3H), 1.61 (br s, 2H), 1.70-1.43 (m, 1H).

### Example 336

### Preparation of methyl (R)-3-(4-amino-5-(3-(5-(difluoromethyl)-3-(2-morpholinoethoxy)thiophene-2-carboxamido)piperidin-1-yl)pyrrolo[2,1-f][1,2,4]triazin-7-yl)-2,5-dihydro-1H-pyrrole-1-carboxylate

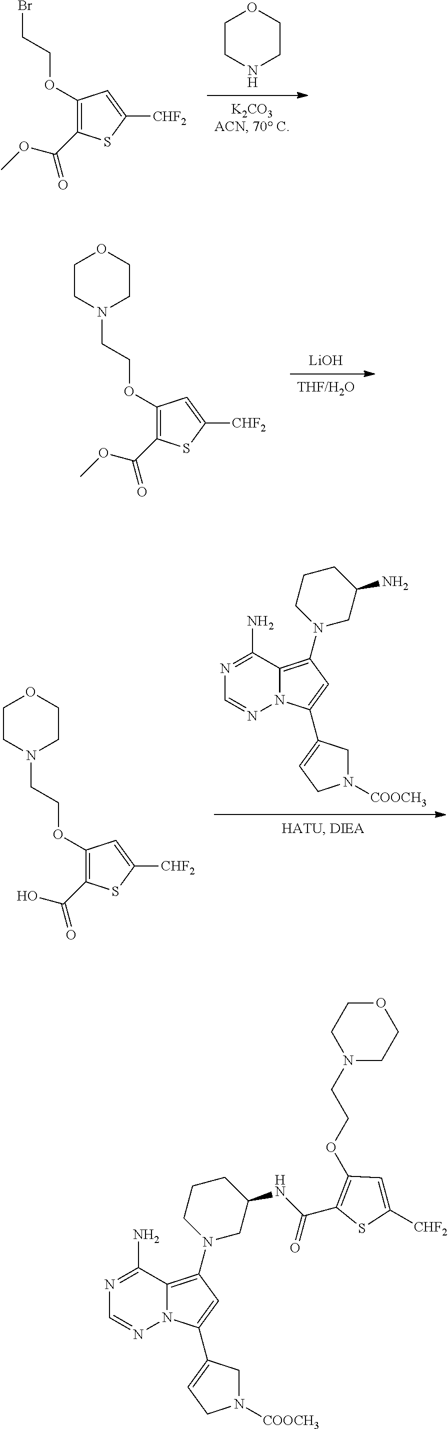

### Example 336A. Methyl 5-(difluoromethyl)-3-(2-morpholinoethoxy)thiophene-2-carboxylate

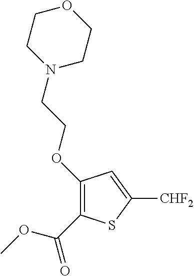 [SMILES: COC(=O)c1sc(C(F)F)cc1OCCN1CCOCC1]

Analogous to the procedure for the preparation of Example 334A, reaction of methyl 3-(2-bromoethoxy)-5-(difluoromethyl)thiophene-2-carboxylate (150 mg, 0.476 mmol) and morpholine (62.2 mg, 0.714 mmol) afforded Example 336A (140 mg, 92% yield) as a yellow oil. MS: [M+H]⁺=322.2.

### Example 336B. 5-(difluoromethyl)-3-(2-morpholinoethoxy)thiophene-2-carboxylic acid

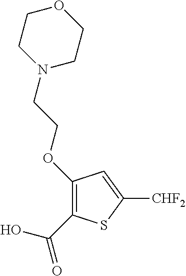 [SMILES: O=C(O)c1sc(C(F)F)cc1OCCN1CCOCC1]

Analogous to the procedure for the preparation of Example 334B, reaction of Example 336A (120 mg, 0.373 mmol) and LiOH (17.89 mg, 0.747 mmol) afforded Example 336B (110 mg, 96% yield) as an off-white solid. MS: [M+H]⁺=308.3.

### Example 336

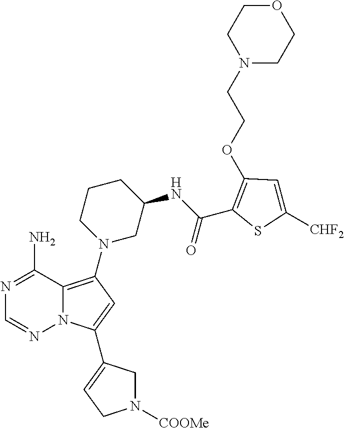 [SMILES: COC(=O)N1CC=C(c2cc(N3CCC[C@@H](NC(=O)c4sc(C(F)F)cc4OCCN4CCOCC4)C3)c3c(N)ncnn23)C1]

Analogous to the procedure for the preparation of Example 334, reaction of Example 336B (43.0 mg, 0.140 mmol) and Intermediate 31 (50 mg, 0.140 mmol) afforded Example 336 (31.3 mg, 35% yield). LC-MS Method E: RT=1.631 min, [M+H]⁺=647.2; LC-MS Method F: RT=1.033 min, [M+H]⁺=647.2; ¹H NMR (400 MHz, DMSO-d₆) δ ppm 8.00 (bs, 1H), 7.86 (s, 1H), 7.61 (br s, 1H), 7.54 (s, 1H), 7.25 (t, J=54.3 Hz, 1H), 6.90 (s, 1H), 6.74 (dd, J=1.3, 3.5 Hz, 2H), 4.59-4.47 (m, 2H), 4.43-4.25 (m, 4H), 4.25-4.09 (m, 1H), 3.66 (d, J=4.4 Hz, 4H), 3.56-3.45 (m, 4H), 3.18-3.07 (m, 1H), 3.01-2.76 (m, 4H), 2.73-2.65 (m, 2H), 2.43-2.34 (m, 3H), 1.95-1.73 (m, 3H).

### Example 337

### Preparation of (R)—N-(1-(4-amino-7-(2-(difluoromethyl)pyridin-4-yl)pyrrolo[2,1-f][1,2,4]triazin-5-yl)piperidin-3-yl)-5-(difluoromethyl)-3-(2-morpholinoethoxy)thiophene-2-carboxamide

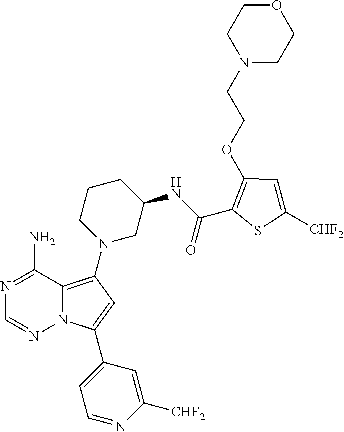 [SMILES: Nc1ncnn2c(-c3ccnc(C(F)F)c3)cc(N3CCC[C@@H](NC(=O)c4sc(C(F)F)cc4OCCN4CCOCC4)C3)c12]

Analogous to the procedure for the preparation of Example 334, reaction of Example (42.8 mg, 0.139 mmol) and Intermediate 33 (50 mg, 0.139 mmol) afforded Example 337 (7 mg, 8% yield). LC-MS Method G: RT=1.608 min, [M+H]⁺=649.4; ¹H NMR (400 MHz, DMSO-d₆) δ ppm 8.73-8.63 (m, 1H), 8.47 (s, 1H), 8.35-8.27 (m, 1H), 8.26-8.16 (m, 1H), 7.99 (s, 1H), 7.70-7.59 (m, 1H), 7.54 (s, 1H), 7.45 (s, 1H), 7.25 (s, 1H), 6.97 (s, 2H), 4.45-4.31 (m, 2H), 4.29-4.15 (m, 1H), 3.55-3.44 (m, 4H), 3.08-2.79 (m, 2H), 2.75-2.62 (m, 4H), 2.33 (br s, 4H), 1.99-1.81 (m, 3H), 1.67-1.53 (m, 1H).

### Example 339

### Preparation of (R)—N-(1-(4-amino-7-(2-(difluoromethyl)pyridin-4-yl)pyrrolo[2,1-f][1,2,4]triazin-5-yl)piperidin-3-yl)-5-chloro-3-(2-(dimethylamino)-2-oxoethoxy)thiophene-2-carboxamide

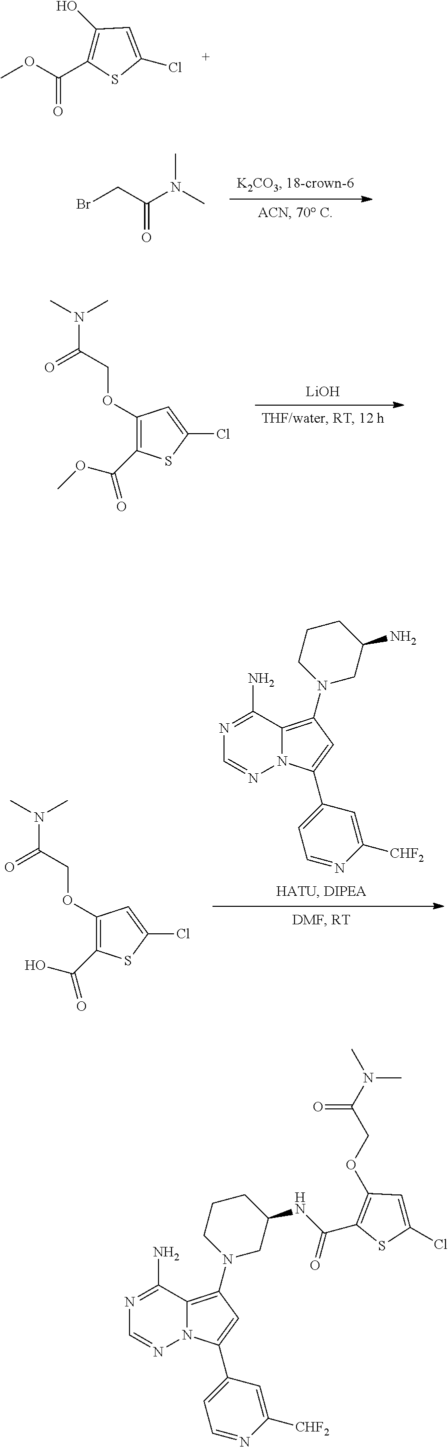

### Example 339A. Methyl 5-chloro-3-(2-(dimethylamino)-2-oxoethoxy)thiophene-2-carboxylate

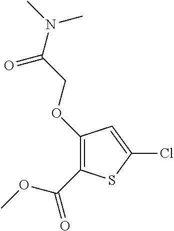 [SMILES: COC(=O)c1sc(Cl)cc1OCC(=O)N(C)C]

Analogous to the procedure for the preparation of Example 324A, reaction of methyl 5-chloro-3-hydroxythiophene-2-carboxylate (200 mg, 1.04 mmol) and 2-bromo-N,N-dimethylacetamide (207 mg, 1.246 mmol) afforded Example 339A (200 mg, 69% yield) as an off-white solid. MS: [M+H]⁺=278.1.

### Example 339B. 5-chloro-3-(2-(dimethylamino)-2-oxoethoxy)thiophene-2-carboxylic acid

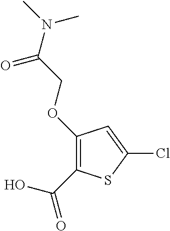 [SMILES: CN(C)C(=O)COc1cc(Cl)sc1C(=O)O]

Analogous to the procedure for the preparation of Example 324B, reaction of Example 339A (200 mg, 0.72 mmol) and LiOH (34.5 mg, 1.44 mmol) afforded Example 339B (200 mg, 69% yield) as an off-white solid. MS: [M+Na]⁺=286.1.

### Example 339

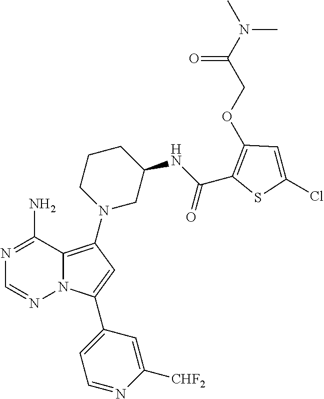 [SMILES: CN(C)C(=O)COc1cc(Cl)sc1C(=O)N[C@@H]1CCCN(c2cc(-c3ccnc(C(F)F)c3)n3ncnc(N)c23)C1]

Analogous to the procedure for the preparation of Example 284, reaction of (R)-5-(3-aminopiperidin-1-yl)-7-(2-(difluoromethyl)pyridin-4-yl)pyrrolo[2,1-f][1,2,4]triazin-4-amine (40 mg, 0.111 mmol) and Example 339B (29.3 mg, 0.111 mmol) Example 339 (7.1 mg, 10% yield). LC-MS Method E: RT=1.723 min, [M+H]⁺=605.2; LC-MS Method F: RT=1.445 min, [M+H]⁺=605.2; ¹H NMR (400 MHz, DMSO-d₆) δ ppm 8.68 (d, J=5.3 Hz, 1H), 8.47 (s, 2H), 8.30 (d, J=5.8 Hz, 1H), 8.12 (br s, 1H), 7.98 (s, 1H), 7.47 (s, 1H), 7.35 (s, 1H), 6.96 (t, J=55.5 Hz, 2H), 5.07 (s, 2H), 4.19 (br dd, J=4.8, 8.5 Hz, 1H), 3.90 (s, 1H), 3.24-3.17 (m, 1H), 2.91 (s, 6H), 2.80 (br s, 3H), 1.91 (s, 3H), 1.69-1.54 (m, 1H).

### Example 340

### Preparation of (R)—N-(1-(4-amino-7-(2-(difluoromethyl)pyridin-4-yl)pyrrolo[2,1-f][1,2,4]triazin-5-yl)piperidin-3-yl)-5-chloro-3-(2-(dimethylamino)ethoxy)thiophene-2-carboxamide

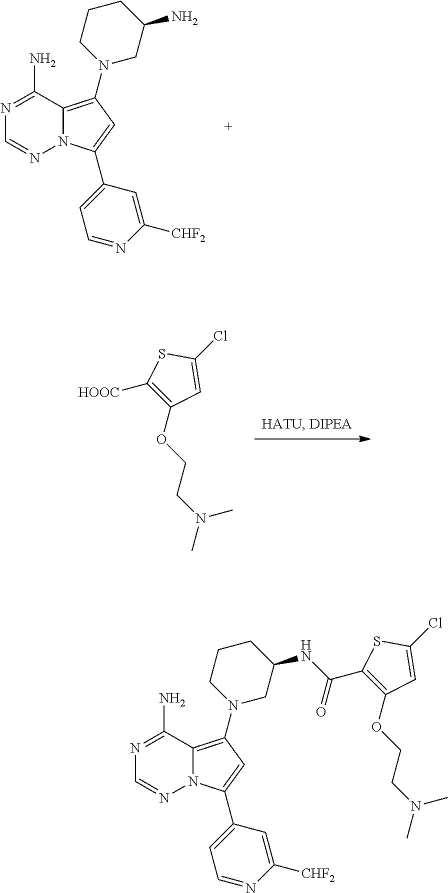 [SMILES: Nc1ncnn2c(-c3ccnc(C(F)F)c3)cc(N3CCC[C@@H](N)C3)c12 ; CN(C)CCOc1cc(Cl)sc1C(=O)O ; "HATU, DIPEA" ; CN(C)CCOc1cc(Cl)sc1C(=O)N[C@@H]1CCCN(c2cc(-c3ccnc(C(F)F)c3)n3ncnc(N)c23)C1]

Analogous to the procedure for the preparation of Example 284, reaction of Intermediate 33 (40 mg, 0.111 mmol) and Example 276B (27.8 mg, 0.111 mmol) afforded Example 340 (12.4 mg, 19% yield). LC-MS Method E: RT=1.867 min, [M+H]⁺=591.1; ¹H NMR (400 MHz, DMSO-d₆) δ ppm 8.69 (d, J=5.4 Hz, 1H), 8.48 (s, 1H), 8.31 (d, J=5.1 Hz, 1H), 8.26-8.11 (m, 1H), 7.99 (s, 1H), 7.74 (br d, J=6.8 Hz, 1H), 7.47 (s, 1H), 7.37 (s, 1H), 6.98 (t, J=56.0 Hz, 2H), 4.30 (t, J=5.1 Hz, 2H), 4.18 (br d, J=2.0 Hz, 1H), 3.17 (d, J=4.9 Hz, 2H), 3.08-2.95 (m, 1H), 2.76 (br s, 1H), 2.66-2.54 (m, 2H), 2.19 (br s, 6H), 1.91 (s, 3H), 1.50 (br s, 1H).

### Example 341

### Preparation of (R)—N-(1-(4-amino-7-(2-(difluoromethyl)pyridin-4-yl)pyrrolo[2,1-f][1,2,4]triazin-5-yl)piperidin-3-yl)-5-chloro-3-(2-morpholinoethoxy)thiophene-2-carboxamide

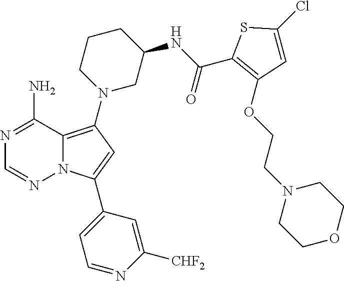 [SMILES: Nc1ncnn2c(-c3ccnc(C(F)F)c3)cc(N3CCC[C@@H](NC(=O)c4sc(Cl)cc4OCCN4CCOCC4)C3)c12]

Analogous to the procedure for the preparation of Example 284, reaction of 5-chloro-3-(2-morpholinoethoxy)thiophene-2-carboxylic acid (32.5 mg, 0.111 mmol) and Intermediate 33 (40 mg, 0.11 mmol) afforded Example 341 (8.8 mg, 11% yield). LC-MS Method E: RT=1.784 min, [M+H]⁺=633.1; ¹H NMR (400 MHz, DMSO-d₆) δ ppm 8.69 (d, J=5.3 Hz, 1H), 8.48 (s, 1H), 8.30 (d, J=5.3 Hz, 1H), 8.20 (br s, 1H), 7.99 (s, 1H), 7.45 (s, 2H), 7.35 (s, 1H), 6.97 (t, J=57.3 Hz, 2H), 4.40-4.28 (m, 2H), 4.20 (br d, J=3.8 Hz, 1H), 3.49 (br s, 3H), 3.18 (s, 2H), 3.02 (br dd, J=4.1, 6.6 Hz, 1H), 2.93-2.78 (m, 2H), 2.75 (s, 1H), 2.68 (br s, 2H), 2.44-2.28 (m, 4H), 1.96-1.79 (m, 2H), 1.73-1.50 (m, 1H).

### Example 342

### Preparation of (R)—N-(1-(4-amino-7-(2-(difluoromethyl)pyridin-4-yl)pyrrolo[2,1-f][1,2,4]triazin-5-yl)piperidin-3-yl)-5-chloro-3-(2-(4-methylpiperazin-1-yl)ethoxy)thiophene-2-carboxamide

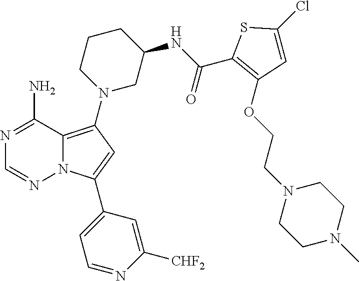 [SMILES: CN1CCN(CCOc2cc(Cl)sc2C(=O)N[C@@H]2CCCN(c3cc(-c4ccnc(C(F)F)c4)n4ncnc(N)c34)C2)CC1]

Analogous to the procedure for the preparation of Example 284, reaction of 5-chloro-3-(2-(4-methylpiperazin-1-yl)ethoxy)thiophene-2-carboxylic acid (33.9 mg, 0.111 mmol) and Intermediate 33 (40 mg, 0.11 mmol) afforded Example 342 (14.2 mg, 20% yield). LC-MS Method E: RT=1.559 min, [M+H]⁺=646.2; ¹H NMR (400 MHz, DMSO-d₆) δ ppm 8.70 (d, J=5.4 Hz, 1H), 8.46 (s, 1H), 8.30 (d, J=5.4 Hz, 1H), 8.19 (br s, 1H), 7.99 (s, 1H), 7.44 (s, 2H), 7.34 (s, 1H), 7.15-6.78 (m, 2H), 4.40-4.25 (m, 2H), 4.25-4.13 (m, 1H), 3.91 (s, 1H), 3.19 (br dd, J=3.8, 7.7 Hz, 1H), 3.07-2.77 (m, 3H), 2.73-2.59 (m, 3H), 2.47-2.09 (m, 8H), 2.01 (s, 3H), 1.95-1.80 (m, 2H), 1.57 (br s, 1H).

### Example 343

### Preparation of (R)—N-(1-(4-amino-7-(2-(difluoromethyl)pyridin-4-yl)pyrrolo[2,1-f][1,2,4]triazin-5-yl)piperidin-3-yl)-5-(difluoromethyl)-3-(2-(dimethylamino)ethoxy)thiophene-2-carboxamide

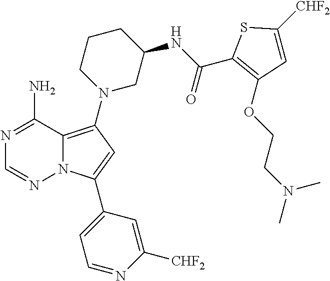 [SMILES: CN(C)CCOc1cc(C(F)F)sc1C(=O)N[C@@H]1CCCN(c2cc(-c3ccnc(C(F)F)c3)n3ncnc(N)c23)C1]

Analogous to the procedure for the preparation of Example 284, reaction of Example 280E (73.8 mg, 0.278 mmol) and Intermediate 33 (100 mg, 0.278 mmol) afforded Example 343 (44 mg, 25% yield). LC-MS Method G: RT=1.74 min, [M+H]⁺=607.4; ¹H NMR (400 MHz, DMSO-d₆) δ ppm 8.69 (d, J=5.4 Hz, 1H), 8.48 (s, 1H), 8.36-8.08 (m, 2H), 7.98 (s, 1H), 7.92-7.80 (m, 2H), 7.57-7.53 (m, 1H), 7.46 (s, 1H), 7.41-7.09 (m, 1H), 6.97 (s, 1H), 4.38-4.29 (m, 2H), 4.26-4.15 (m, 1H), 3.25-2.95 (m, 5H), 2.92-2.69 (m, 2H), 2.19 (br s, 6H), 1.90-1.79 (m, 2H), 1.62-1.40 (m, 1H).

### Example 344

### Preparation of (R)—N-(1-(4-amino-7-(2-(difluoromethyl)pyridin-4-yl)pyrrolo[2,1-f][1,2,4]triazin-5-yl)piperidin-3-yl)-5-(difluoromethyl)-3-morpholinothiophene-2-carboxamide

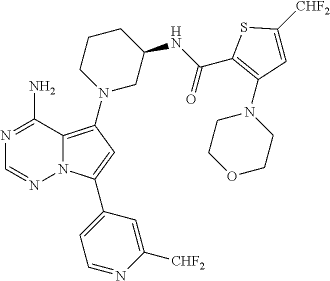 [SMILES: Nc1ncnn2c(-c3ccnc(C(F)F)c3)cc(N3CCC[C@@H](NC(=O)c4sc(C(F)F)cc4N4CCOCC4)C3)c12]

Analogous to the procedure for the preparation of Example 284, reaction of Example 318B (29.3 mg, 0.111 mmol) and Intermediate 33 (40 mg, 0.111 mmol) afforded Example 344 (6.5 mg, 10% yield). LC-MS Method E: RT=1.810 min, [M+H]⁺=605.2; LC-MS Method F: RT=1.517 min, [M+H]⁺=605.3; ¹H NMR (400 MHz, DMSO-d₆) δ ppm 9.08-8.95 (m, 1H), 8.69 (d, J=5.5 Hz, 1H), 8.48 (s, 1H), 8.30 (d, J=5.3 Hz, 1H), 8.26-8.08 (m, 1H), 7.99 (s, 1H), 7.67 (s, 1H), 7.48 (s, 1H), 7.26 (t, J=55.5 Hz, 1H), 6.97 (t, J=55.3 Hz, 2H), 4.28-4.13 (m, 1H), 3.75 (br t, J=4.1 Hz, 4H), 3.08-3.00 (m, 1H), 2.95 (br d, J=3.3 Hz, 4H), 2.89-2.72 (m, 2H), 2.06-1.95 (m, 1H), 1.94-1.83 (m, 3H), 1.65-1.47 (m, 1H).

### Example 345

### Preparation of (R)—N-(1-(4-amino-7-(2-(difluoromethyl)pyridin-4-yl)pyrrolo[2,1-f][1,2,4]triazin-5-yl)piperidin-3-yl)-5-(difluoromethyl)-3-(2-oxa-6-azaspiro[3.3]heptan-6-yl)thiophene-2-carboxamide

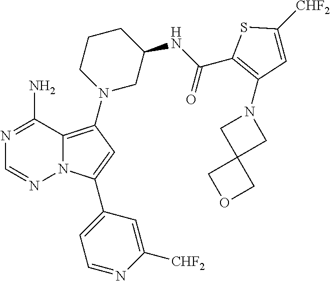 [SMILES: Nc1ncnn2c(-c3ccnc(C(F)F)c3)cc(N3CCC[C@@H](NC(=O)c4sc(C(F)F)cc4N4CC5(COC5)C4)C3)c12]

Analogous to the procedure for the preparation of Example 284, reaction of Example 3149C (30.6 mg, 0.111 mmol) and Intermediate (40 mg, 0.111 mmol) afforded Example 345 (7.9 mg, 11% yield). LC-MS Method E: RT=1.729 min, [M+H]⁺=617.2; LC-MS Method F: RT=1.430 min, [M+H]⁺=617.2; ¹H NMR (400 MHz, DMSO-d₆) δ ppm 8.70 (d, J=5.4 Hz, 1H), 8.50 (s, 1H), 8.32 (s, 1H), 8.24-8.06 (m, 1H), 8.00 (s, 1H), 7.92 (br d, J=3.2 Hz, 1H), 7.46 (s, 1H), 7.36-6.80 (m, 4H), 4.68-4.57 (m, 4H), 4.17-4.09 (m, 1H), 4.04 (s, 4H), 3.25-3.20 (m, 1H), 3.00 (br s, 1H), 2.86-2.69 (m, 2H), 1.94-1.72 (m, 3H), 1.65-1.50 (m, 1H).

### Example 346

### Preparation of (R)—N-(1-(4-amino-7-(4-((5-chloro-2-fluorophenyl)sulfonamido)phenyl)pyrrolo[2,1-f][1,2,4]triazin-5-yl)piperidin-3-yl)-5-chlorothiophene-2-carboxamide

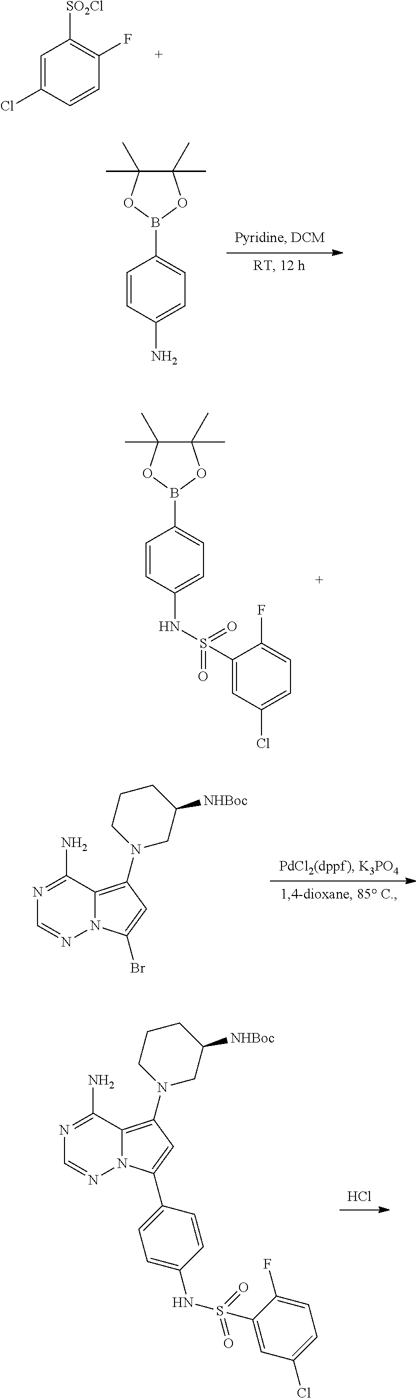

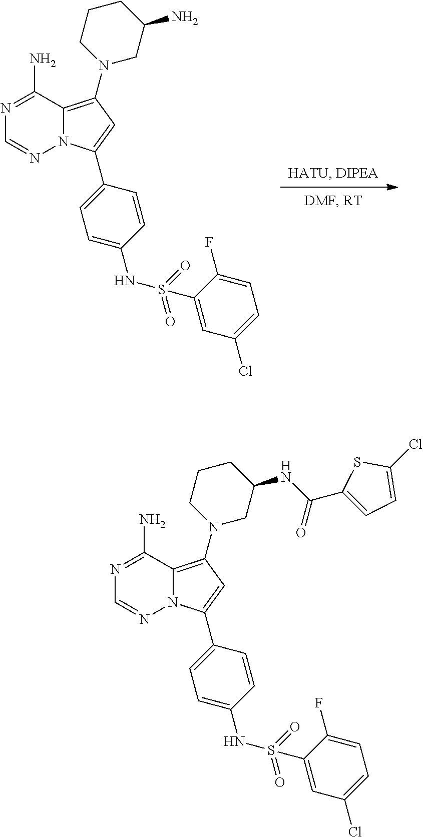

### Example 346A. 5-chloro-2-fluoro-N-(4-(4,4,5,5-tetramethyl-1,3,2-dioxaborolan-2-yl)phenyl)benzenesulfonamide

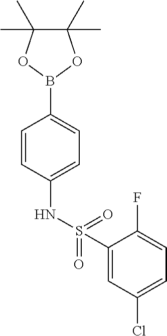 [SMILES: CC1(C)OB(c2ccc(NS(=O)(=O)c3cc(Cl)ccc3F)cc2)OC1(C)C]

Pyridine (0.318 mL, 3.93 mmol) was added to a solution of 5-chloro-2-fluorobenzenesulfonyl chloride (300 mg, 1.310 mmol) and 4-(4,4,5,5-tetramethyl-1,3,2-dioxaborolan-2-yl)aniline (287 mg, 1.310 mmol) in DCM (6 mL) at rt. After stirring at rt for 12 h, mixture was partitioned between water (50 mL) and chloroform (20 mL). The organic layer was separated, dried over sodium sulphate and concentrated under reduced pressure to afford Example 346A (500 mg, 93% yield) as an orange solid. MS: [M+H]⁺=410.4.

### Example 346B. tert-butyl (R)-(1-(4-amino-7-(4-((5-chloro-2-fluorophenyl)sulfonamido)phenyl)pyrrolo[2,1-f][1,2,4]triazin-5-yl)piperidin-3-yl)carbamate

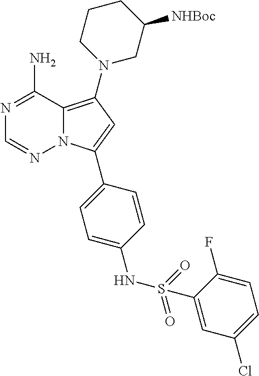 [SMILES: CC(C)(C)OC(=O)N[C@@H]1CCCN(c2cc(-c3ccc(NS(=O)(=O)c4cc(Cl)ccc4F)cc3)n3ncnc(N)c23)C1]

PdCl₂(dppf)-DCM adduct (59.6 mg, 0.073 mmol) was added to a solution of tert-butyl (R)-(1-(4-amino-7-bromopyrrolo[2,1-f][1,2,4]triazin-5-yl)piperidin-3-yl)carbamate (300 mg, 0.729 mmol), Example 346A (300 mg, 0.729 mmol) and tripotassium phosphate (2M, 1.09 mL, 2.19 mmol) in dioxane (6 mL). The reaction mixture was heated at 85° C. for 12 h, cooled to rt and partitioned between water (50 mL) and ethyl acetate (20 mL). The organic layer was separated, dried over sodium sulphate and concentrated under reduced pressure to afford Example 346B (400 mg, 89% yield) as a yellow solid. MS: [M+H]⁺=616.4.

### Example 346C. (R)—N-(4-(4-amino-5-(3-aminopiperidin-1-yl)pyrrolo[2,1-f][1,2,4]triazin-7-yl)phenyl)-5-chloro-2-fluorobenzenesulfonamide

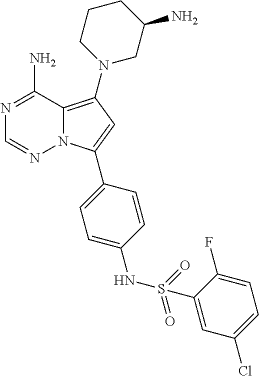 [SMILES: Nc1ncnn2c(-c3ccc(NS(=O)(=O)c4cc(Cl)ccc4F)cc3)cc(N3CCC[C@@H](N)C3)c12]

To a solution of Example 346B (300 mg, 0.487 mmol) in dioxane, was added HCl (5 mL, 4N) in dioxane. The reaction mixture was stirred for 2 h and concentrated to afford Example 346C (200 mg, 80% yield) as a grey solid. MS: [M+H]⁺=516.3.

### Example 346

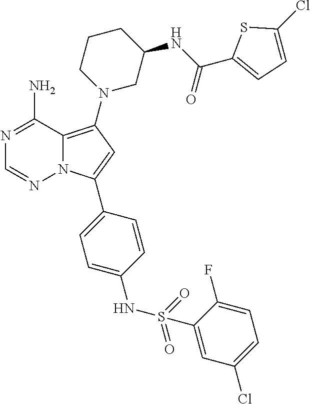 [SMILES: Nc1ncnn2c(-c3ccc(NS(=O)(=O)c4cc(Cl)ccc4F)cc3)cc(N3CCC[C@@H](NC(=O)c4ccc(Cl)s4)C3)c12]

DIEA (0.051 mL, 0.291 mmol) was added to a solution of Example 346C (50 mg, 0.097 mmol), 5-chlorothiophene-2-carboxylic acid (15.75 mg, 0.097 mmol) and HATU (36.8 mg, 0.097 mmol) in DMF (2 mL). The reaction mixture was stirred at rt for 2 h, evaporated under reduced pressure and purified by preparative HPLC to afford Example 346 (1.8 mg, 2.6% yield). LC-MS Method E: RT=2.011 min, [M+H]⁺=661.2; LC-MS Method F: RT=1.744 min, [M+H]⁺=661.2.

### Example 347

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(piperidin-4-yloxy)thiophene-2-carboxamide

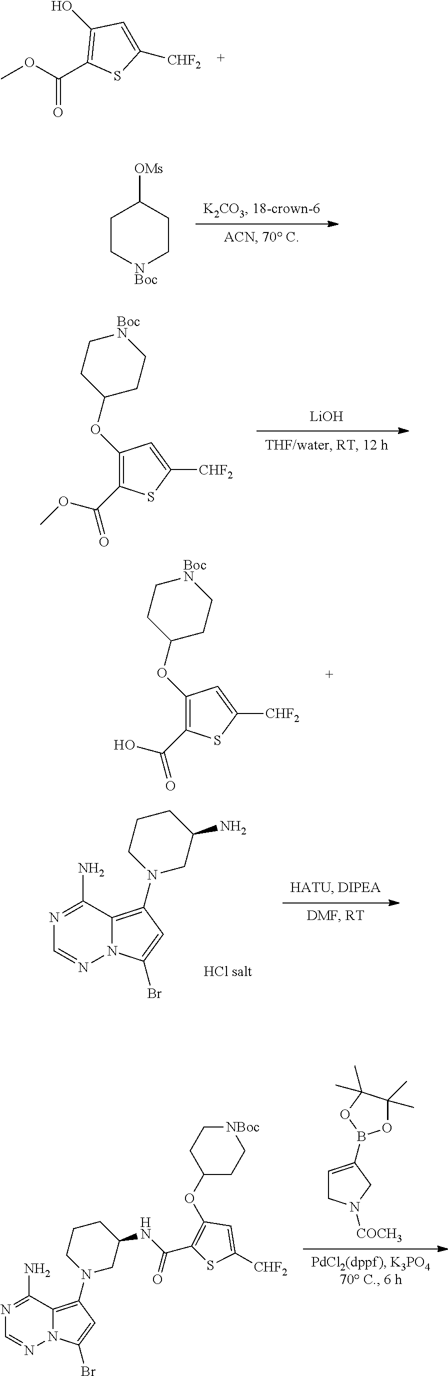

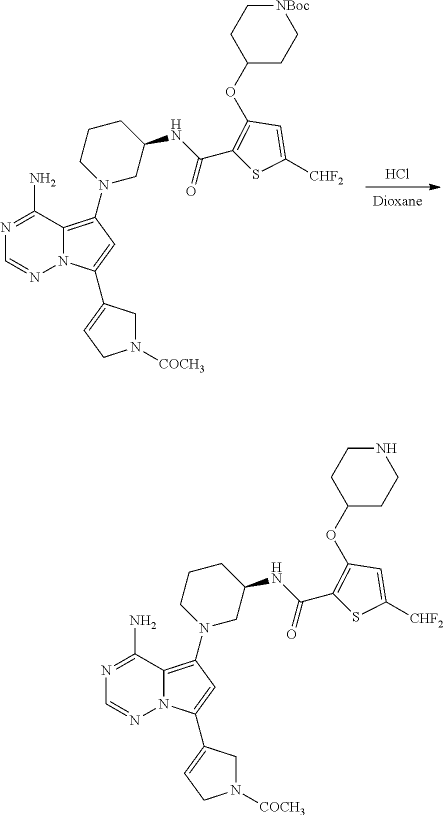

### Example 347A. tert-butyl 4-((5-(difluoromethyl)-2-(methoxycarbonyl)thiophen-3-yl)oxy)piperidine-1-carboxylate

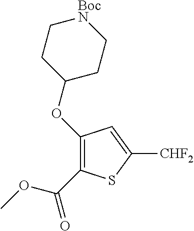 [SMILES: COC(=O)c1sc(C(F)F)cc1OC1CCN(C(=O)OC(C)(C)C)CC1]

A solution of methyl 5-(difluoromethyl)-3-hydroxythiophene-2-carboxylate (500 mg, 2.402 mmol), K₂CO₃ (1 g, 6.6 mmol), 18-crown-6 (63 mg, 0.24 mmol) and tert-butyl 4-((methylsulfonyl)oxy)piperidine-1-carboxylate (1342 mg, 4.80 mmol) were taken in acetonitrile (12 mL) and heated at 70° C. for 14 h. After cooling, reaction mixture was diluted with ethyl acetate (50 mL) and washed with water (50 mL). The organic layer was separated, dried over sodium sulphate and concentrated under reduced pressure. The residue was purified by flash chromatography to afford Example 347A (900 mg, 96% yield) as a yellow gummy solid. MS: [M−^{t}Bu]⁺=336.2.

### Example 347B. 3-((1-(tert-butoxycarbonyl)piperidin-4-yl)oxy)-5-(difluoromethyl)thiophene-2-carboxylic acid

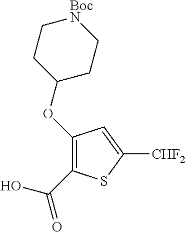 [SMILES: CC(C)(C)OC(=O)N1CCC(Oc2cc(C(F)F)sc2C(=O)O)CC1]

Analogous to the procedure for the preparation of Example 314C, reaction of Example 347A (700 mg, 1.788 mmol) and LiOH (86 mg, 3.58 mmol) afforded Example 347B (600 mg, 89% yield) as an off-white solid. MS: [M−Boc+2H]⁺=278.2.

### Example 347C. tert-butyl (R)-4-((2-((1-(4-amino-7-bromopyrrolo[2,1-f][1,2,4]triazin-5-yl)piperidin-3-yl)carbamoyl)-5-(difluoromethyl)thiophen-3-yl)oxy)piperidine-1-carboxylate

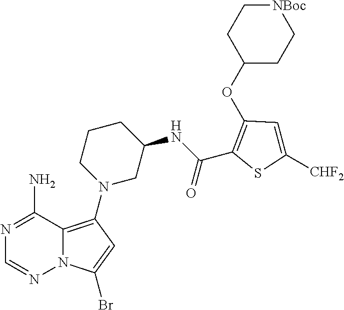 [SMILES: CC(C)(C)OC(=O)N1CCC(Oc2cc(C(F)F)sc2C(=O)N[C@@H]2CCCN(c3cc(Br)n4ncnc(N)c34)C2)CC1]

DIEA (0.674 mL, 3.86 mmol) was added to a solution of (R)-5-(3-aminopiperidin-1-yl)-7-bromopyrrolo[2,1-f][1,2,4]triazin-4-amine (400 mg, 1.29 mmol), Example 347B (485 mg, 1.29 mmol) and HATU (489 mg, 1.29 mmol) in DMF (1 mL). Stirred at rt for 2 h, diluted with ethyl acetate (50 mL) and washed with water (50 mL). The organic layer was separated, dried over sodium sulphate and concentrated under reduced pressure. The residue was purified by flash chromatography (0-7% DCM/MeOH gradient) to afford Example 347C (400 mg, 46% yield) as a yellow solid. MS: [M+H]⁺=672.4.

### Example 347D. tert-butyl (R)-4-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-(difluoromethyl)thiophen-3-yl)oxy)piperidine-1-carboxylate

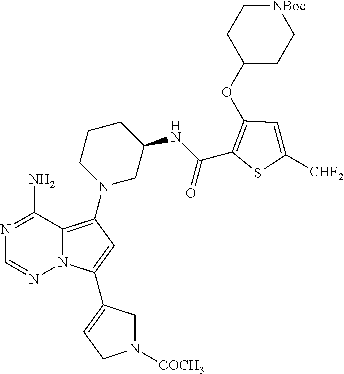 [SMILES: CC(=O)N1CC=C(c2cc(N3CCC[C@@H](NC(=O)c4sc(C(F)F)cc4OC4CCN(C(=O)OC(C)(C)C)CC4)C3)c3c(N)ncnn23)C1]

PdCl₂(dppf)-DCM adduct (8.52 mg, 10.44 μmol) was added to a solution of Example 347C (70 mg, 0.104 mmol), I-(3-(4,4,5,5-tetramethyl-1,3,2-dioxaborolan-2-yl)-2,5-dihydro-1H-pyrrol-1-yl)ethan-1-one (37.1 mg, 0.157 mmol) and tripotassium phosphate (0.157 mL 2M solution, 0.313 mmol) in THF (3 mL). The reaction mixture was heated at 75° C. for 4 h. After cooling, reaction mixture was partitioned between water (50 mL) and ethyl acetate (50 mL). The organic layer was separated, dried over sodium sulphate and concentrated under reduced pressure. The residue was purified by flash chromatography (0-10% DCM/MeOH) to afford Example 347D (60 mg, 82% yield) as a yellow semi solid. MS: [M+H]⁺=701.5.

### Example 347

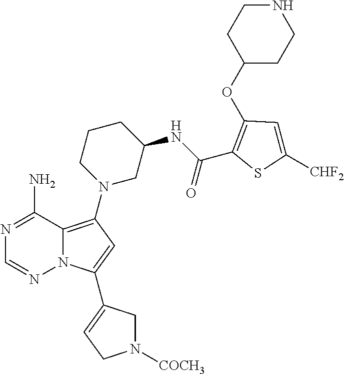 [SMILES: CC(=O)N1CC=C(c2cc(N3CCC[C@@H](NC(=O)c4sc(C(F)F)cc4OC4CCNCC4)C3)c3c(N)ncnn23)C1]

To a suspension of Example 347D (60 mg, 0.086 mmol) in dioxane, HCl (1 mL, 4N) in dioxane was added and stirred at rt for 4 h. The reaction mixture was evaporated to dryness and purified by preparative HPLC to afford Example 347 (7.9 mg, 11% yield). LC-MS Method E: RT=1.19 min, [M+H]⁺=601.2; LC-MS Method F: RT=0.92 min. [M+H]⁺=601.2; ¹H NMR (400 MHz, DMSO-d₆) δ ppm 7.86 (d, J=0.8 Hz, 1H), 7.58 (s, 2H), 7.23 (t, J=53.5 Hz, 1H), 6.93 (bs, 1H), 6.82-6.64 (m, 3H), 4.75-4.62 (m, 2H), 4.52-4.44 (m, 3H), 4.29-4.25 (m, 1H), 4.20 (br dd, J=1.9, 3.6 Hz, 1H), 3.07-2.98 (m, 4H), 2.88-2.73 (m, 4H), 2.15-1.98 (m, 4H), 1.95-1.77 (m, 5H), 1.72-1.50 (m, 1H).

The following Examples 348 and 349 in Table 20 were prepared using the same procedure as shown in Example 347. Example 347C was coupled with the appropriate boronic acid or boronate ester and followed by Boc-deprotection using 4N HCl in dioxane.

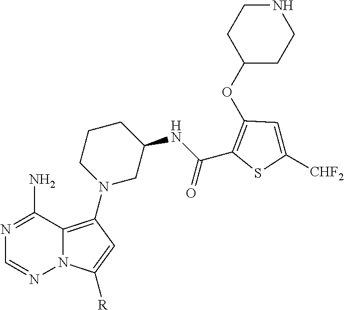

TABLE 20

| | | | | HPLC | |
|---|---|---|---|---|---|
| | Structure | | LCMS | Method, | |
| Example | (R) | Name | (M + H)⁺ | RT (min.) | ¹H NMR |
| 348 | 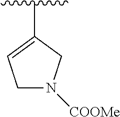 [SMILES: *C1=CCN(C(=O)OC)C1] | methyl (R)-3-(4-amino-5-(3-(5- (difluoromethyl)-3-(piperidin-4- yloxy)thiophene-2- carboxamido)piperidin-1- yl)pyrrolo[2,1-f][1,2,4]triazin-7- yl)-2,5-dihydro-1H-pyrrole-1- carboxylate | 617.30 | E: 1.36 F: 1.05 | (400 MHz, DMSO-d6) δ ppm 7.90 (s, 1H), 7.59 (s, 1H), 7.51-7.40 (m, 2H), 7.40-6.92 (m, 2H), 6.76 (dd, J = 2.1, 6.4 Hz, 2H), 4.83-4.76 (m, 1H), 4.59-4.47 (m, 2H), 4.25-4.09 (m, 1H), 3.68-3.63 (m, 4H), 3.29-3.17 (m, 3H), 3.12-2.96 (m, 4H), 2.87-2.73 (m, 2H), 2.25-2.10 (m, 2H), 1.99-1.72 (m, 5H), 1.60-1.44 (m, 1H) |
| 349 | 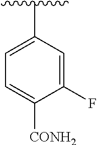 [SMILES: *c1ccc(C(N)=O)c(F)c1] | (R)-N-(1-(4-amino-7-(4- carbamoyl-3- fluorophenyl)pyrrolo[2,1- f][1,2,4]triazin-5-yl)piperidin-3- yl)-5-(difluoromethyl)-3- (piperidin-4-yloxy)thiophene-2- carboxamide | 629.2 | E: 1.20 F: 0.93 | (400 MHz, DMSO-d6) δ ppm 8.13 (dd, J = 1.5, 13.2 Hz, 1H), 8.01 (dd, J = 1.6, 8.2 Hz, 1H), 7.92 (s, 1H), 7.75 (t, J = 8.1 Hz, 1H), 7.69-7.54 (m, 4H), 7.39-7.04 (m, 2H), 6.88 (s, 1H), 4.67 (td, J = 4.3, 8.4 Hz, 1H), 4.23 (br dd, J = 3.4, 6.1 Hz, 1H), 3.09-2.80 (m, 6H), 2.72-2.66 (m, 2H), 2.08-1.98 (m, 2H), 1.95-1.78 (m, 4H), 1.70-1.49 (m, 3H) |

### Example 350

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(piperidin-4-ylmethoxy)thiophene-2-carboxamide

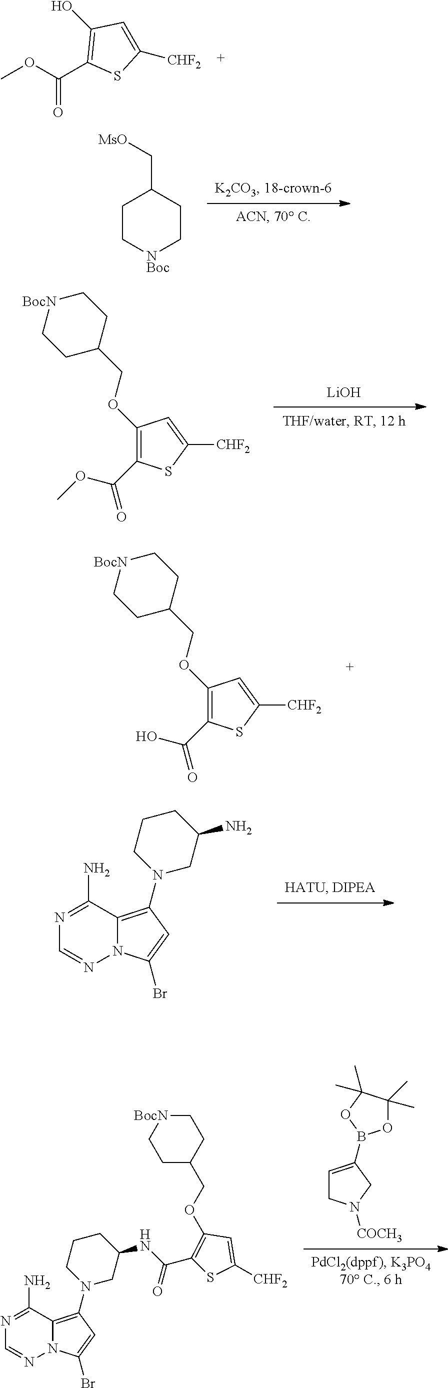

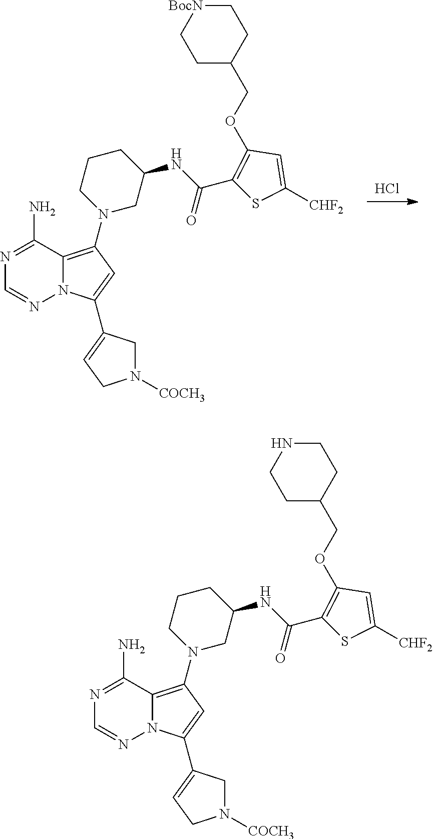 [SMILES: CC(=O)N1CC=C(c2cc(N3CCC[C@@H](NC(=O)c4sc(C(F)F)cc4OCC4CCN(C(=O)OC(C)(C)C)CC4)C3)c3c(N)ncnn23)C1 ; Cl ; CC(=O)N1CC=C(c2cc(N3CCC[C@@H](NC(=O)c4sc(C(F)F)cc4OCC4CCNCC4)C3)c3c(N)ncnn23)C1]

### Example 350A. tert-butyl 4-(((5-(difluoromethyl)-2-(methoxycarbonyl)thiophen-3-yl)oxy)methyl)piperidine-1-carboxylate

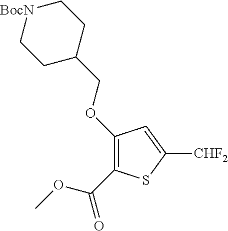 [SMILES: COC(=O)c1sc(C(F)F)cc1OCC1CCN(C(=O)OC(C)(C)C)CC1]

Analogous to the procedure for the preparation of Example 347A, reaction of methyl 5-(difluoromethyl)-3-hydroxythiophene-2-carboxylate (300 mg, 1.441 mmol) and tert-butyl 4-(((methylsulfonyl)oxy)methyl)piperidine-1-carboxylate (846 mg, 2.88 mmol) afforded Example 350A (500 mg, 89% yield) as a yellow gummy solid. MS: [M−Boc+2H]⁺=306.2.

### Example 350B. 3-((1-(tert-butoxycarbonyl)piperidin-4-yl)methoxy)-5-(difluoromethyl)thiophene-2-carboxylic acid

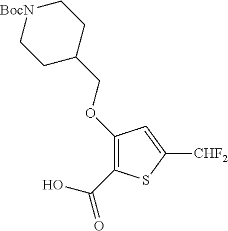 [SMILES: CC(C)(C)OC(=O)N1CCC(COc2cc(C(F)F)sc2C(=O)O)CC1]

Analogous to the procedure for the preparation of Example 314C, reaction of Example 350A (600 mg, 1.480 mmol) and LiOH (70.9 mg, 2.96 mmol) afforded Example 350B (520 mg, 90% yield) as an off-white solid. MS: [M+H]⁺=391.3.

### Example 350C. tert-butyl (R)-4-(((2-((1-(4-amino-7-bromopyrrolo[2,1-f][1,2,4]triazin-5-yl)piperidin-3-yl)carbamoyl)-5-(difluoromethyl)thiophen-3-yl)oxy)methyl)piperidine-1-carboxylate

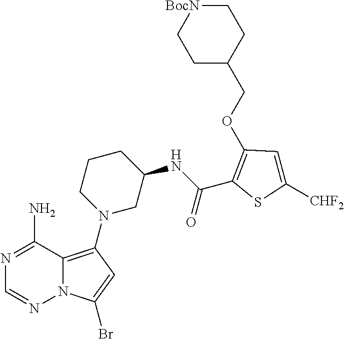 [SMILES: CC(C)(C)OC(=O)N1CCC(COc2cc(C(F)F)sc2C(=O)N[C@@H]2CCCN(c3cc(Br)n4ncnc(N)c34)C2)CC1]

Analogous to the procedure for the preparation of Example 347C, reaction of (R)-5-(3-aminopiperidin-1-yl)-7-bromopyrrolo[2,1-f][1,2,4]triazin-4-amine (400 mg, 1.29 mmol) and Example 350B (503 mg, 1.29 mmol) afforded Example 350C (700 mg, 80% yield) as a yellow solid. MS: [M+H]⁺=686.2.

### Example 350D. tert-butyl (R)-4-(((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-(difluoromethyl)thiophen-3-yl)oxy)methyl)piperidine-1-carboxylate

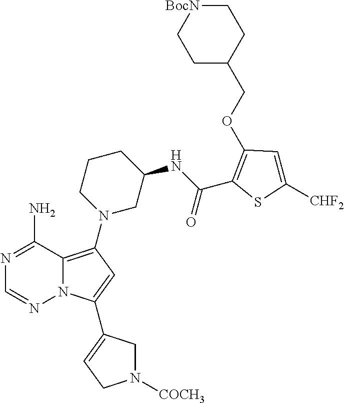 [SMILES: CC(=O)N1CC=C(c2cc(N3CCC[C@@H](NC(=O)c4sc(C(F)F)cc4OCC4CCN(C(=O)OC(C)(C)C)CC4)C3)c3c(N)ncnn23)C1]

Analogous to the procedure for the preparation of Example 347D, reaction of Example 350C and 1-(3-(4,4,5,5-tetramethyl-1,3,2-dioxaborolan-2-yl)-2,5-dihydro-1H-pyrrol-1-yl)ethan-1-one afforded Example 350D (80 mg, 77% yield) as a pale brown solid. MS: [M+H]⁺=715.5.

### Example 350

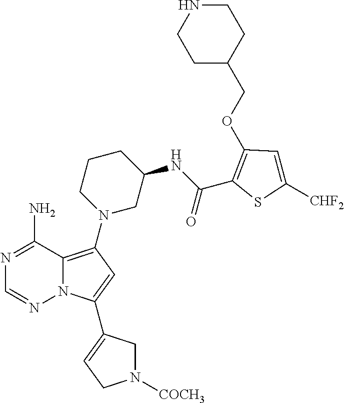 [SMILES: CC(=O)N1CC=C(c2cc(N3CCC[C@@H](NC(=O)c4sc(C(F)F)cc4OCC4CCNCC4)C3)c3c(N)ncnn23)C1]

To a solution of Example 350D (50 mg, 0.070 mmol) in dioxane, 4N HCl (350 μl, 1.40 mmol) in dioxane was added and stirred at rt for 6 h. The reaction mixture was evaporated under reduced pressure and purified by preparative HPLC to afford Example 350 (16 mg, 37% yield). LC-MS Method E: RT=1.19 min, [M+H]⁺=615.2; LC-MS Method F: RT=0.95 min, [M+H]⁺=615.2; ¹H NMR (400 MHz, DMSO-d₆) δ ppm 7.88 (d, J=1.5 Hz, 1H), 7.61-7.50 (m, 3H), 7.25 (t, J=55.0 Hz, 1H), 6.92 (bs, 1H), 6.82-6.74 (m, 1H), 6.72 (d, J=9.5 Hz, 1H), 4.72-4.67 (m, 1H), 4.53-4.46 (m, 2H), 4.29-4.25 (m, 1H), 4.22-4.06 (m, 4H), 3.16-3.10 (m, 1H), 2.99-2.79 (m, 5H), 2.44-2.25 (m, 2H), 2.04 (d, J=16.9 Hz, 3H), 1.92-1.73 (m, 5H), 1.72-1.58 (m, 3H), 1.22-1.11 (m, 1H).

### Example 351

### Preparation of methyl (R)-3-(4-amino-5-(3-(5-(difluoromethyl)-3-(piperidin-4-yl-methoxy)thiophene-2-carboxamido)piperidin-1-yl)pyrrolo[2,1-f][1,2,4]triazin-7-yl)-2,5-dihydro-1H-pyrrole-1-carboxylate

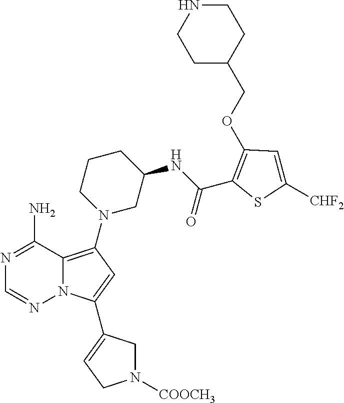 [SMILES: COC(=O)N1CC=C(c2cc(N3CCC[C@@H](NC(=O)c4sc(C(F)F)cc4OCC4CCNCC4)C3)c3c(N)ncnn23)C1]

Analogous to the procedure for the preparation of Example 350, reaction of Example 350C and methyl 3-(4,4,5,5-tetramethyl-1,3,2-dioxaborolan-2-yl)-2,5-dihydro-1H-pyrrole-1-carboxylate, followed by Boc-deprotection using 4N HCl afforded Example 351 (32.8 mg, 48% yield). LC-MS Method E: RT=1.35 min, [M+H]⁺=631.2; LC-MS Method F: RT=1.08 min, [M+H]⁺=631.2; ¹H NMR (400 MHz, DMSO-d₆) δ ppm 7.86 (s, 1H), 7.55 (s, 2H), 7.25 (t, J=53.8 Hz, 1H), 6.90 (bs, 1H), 6.80-6.66 (m, 3H), 4.60-4.49 (m, 2H), 4.35-4.25 (m, 2H), 4.24-4.06 (m, 3H), 3.66 (d, J=4.2 Hz, 3H), 3.17-3.08 (m, 1H), 2.97-2.76 (m, 5H), 2.34 (br d, J=1.7 Hz, 2H), 1.92-1.75 (m, 5H), 1.71-1.58 (m, 3H), 1.24-1.09 (m, 1H).

### Example 352

### (R)—N-(1-(7-(1-acetyl-2,5-dihydro-1H-pyrrol-3-yl)-4-aminopyrrolo[2,1-f][1,2,4]triazin-5-yl)piperidin-3-yl)-4-(2-(dimethylamino)ethoxy)-2-methylthiazole-5-carboxamide

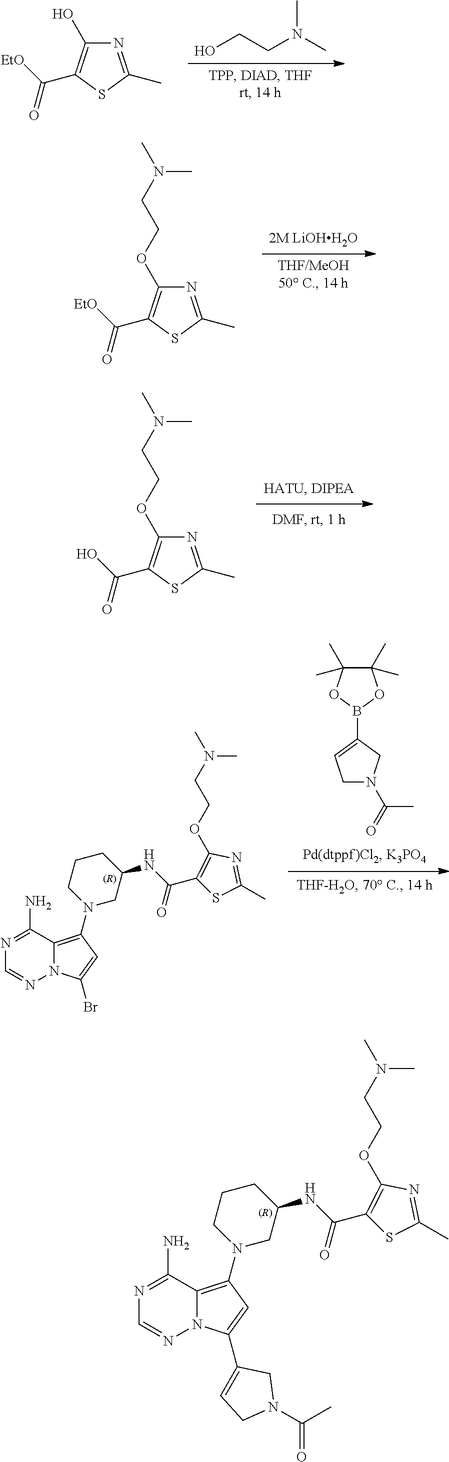

### Example 352A. Ethyl 4-(2-(dimethylamino)ethoxy)-2-methylthiazole-5-carboxylate

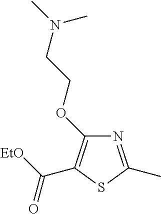 [SMILES: CCOC(=O)c1sc(C)nc1OCCN(C)C]

To a stirred solution of ethyl 4-hydroxy-2-methylthiazole-5-carboxylate (1 g, 5.54 mmol) and 2-(dimethylamino) ethan-1-ol (0.71 g, 8.01 mmol) in THF (10 mL), was added triphenylphosphine (4.2 g, 16.02 mmol). Reaction mixture was stirred for 10 min at rt and then DIAD (1.55 mL, 8.01 mmol) was added slowly. The mixture was allowed to stir at rt for 14 h followed by the addition of water (30 mL) and EtOAc (50 mL). Organic phase was separated, dried over Na₂SO₄, filtered and concentrated. The crude product was purified by silica gel chromatography (4% MeOH in DCM) which afforded Example 352A (1.0 g, 73% yield) as a gummy liquid. MS: [M+H]⁺=259.0; ¹H NMR (400 MHz, CDCl₃) δ ppm 4.57 (t, J=6.0 Hz, 2H), 4.29 (q, J=7.0 Hz, 2H), 2.78 (t, J=5.8 Hz, 2H), 2.67-2.59 (m, 3H), 2.53 (br d, J=8.0 Hz, 1H), 2.40-2.27 (m, 6H), 2.23-2.14 (m, 1H), 1.36-1.31 (m, 3H), 1.29-1.25 (m, 2H).

### Example 352B. 4-(2-(dimethylamino)ethoxy)-2-methylthiazole-5-carboxylic acid. Lithium salt

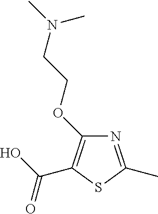

Example 352A (0.9 g, 3.48 mmol) was dissolved in THF (8 mL) and MeOH (1 mL). Lithium hydroxide monohydrate (2M 5 mL aq. solution, 10.45 mmol) was added. The reaction mixture was heated at 50° C. for 14 h. After cooling, mixture was concentrated to dryness, which afforded Example 352B (0.7 g, 87% yield) as an off-white solid. MS: [M+H]⁺=231.2; ¹H NMR (400 MHz, DMSO-d₆) δ ppm 4.29 (t, J=6.0 Hz, 2H), 2.57 (t, J=6.0 Hz, 2H), 2.49 (s, 3H), 2.18 (s, 6H).

### Example 352C. (R)—N-(1-(4-amino-7-bromopyrrolo[2,1-f][1,2,4]triazin-5-yl)piperidin-3-yl)-4-(2-(dimethylamino)ethoxy)-2-methylthiazole-5-carboxamide

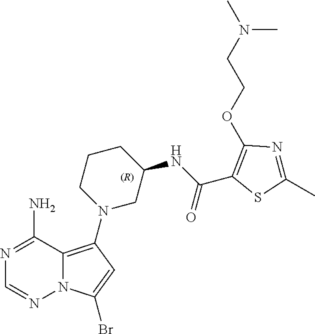

(R)-5-(3-aminopiperidin-1-yl)-7-bromopyrrolo[2,1-f][1,2,4]triazin-4-amine (0.6 g, 1.72 mmol), 4 Example 352B (0.46 g 1.72 mmol) and DIEA (0.9 mL, 5.18 mmol) were taken in anhydrous DMF (5 mL). HATU (0.72 g, 1.89 mmol) was added and the reaction mixture was stirred at rt for 1 h. Reaction mixture was diluted with water (25 mL) and EtOAc (50 mL). Organic phase was separated, dried over Na₂SO₄, filtered and concentrated. Resulted crude product was purified by silica gel chromatography (5% MeOH in DCM) which afforded Example 352C (0.8 g, 89% yield) as a yellow solid. MS: [M+H]⁺=525.2.

### Example 352

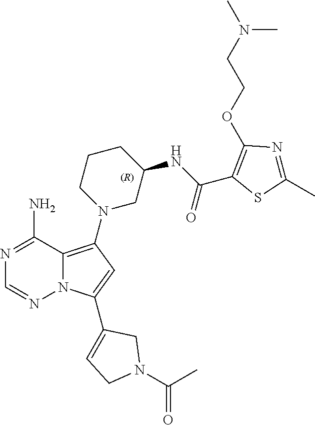

Example 352C (40 mg, 0.076 mmol), 1-(3-(4,4,5,5-tetramethyl-1,3,2-dioxaborolan-2-yl)-2,5-dihydro-1H-pyrrol-1-yl)ethan-1-one (27.2 mg, 0.11 mmol) and K₃PO₄ (32.4 mg, 0.153 mmol) were taken in THF (2 mL) and water (0.2 mL). The mixture was degassed with N₂ and then PdCl₂(dtppf) (3.49 mg, 5.35 μmol) was added followed by heating at 70° C. for 14 h. After cooling water (25 mL) and EtOAc (50 mL) were added. Organic phase was separated, dried over Na₂SO₄, filtered and concentrated. Resulted crude product was purified by HPLC, which afforded Example 352 (22 mg, 48% yield). LC-MS Method E: RT=1.30 min, [M+H]⁺=554.3; LC-MS Method F: RT=0.84 min, [M+H]⁺=554.3; ¹H NMR (400 MHz, DMSO-d₆) δ ppm 8.02 (br s, 1H), 7.87 (d, J=1.0 Hz, 1H), 7.62 (s, 1H), 6.80-6.69 (m, 2H), 4.71-4.66 (m, 1H), 4.52-4.45 (m, 4H), 4.27 (br s, 2H), 4.14 (br s, 1H), 3.15 (br s, 1H), 2.96 (s, 1H), 2.61 (s, 6H), 2.18 (br s, 6H), 2.03 (d, J=16.3 Hz, 3H), 1.86 (br s, 3H), 1.49 (br s, 1H).

The following Examples, 353-356 in Table 21, were prepared using the same procedure as shown in Example 352. Example 352C was coupled with the appropriate Boronic acids or Boronate esters.

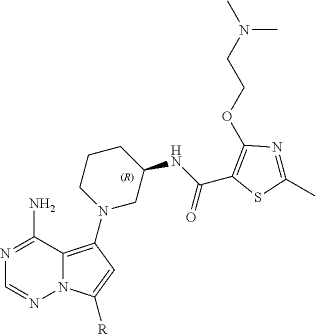

TABLE 21

| | | | | HPLC | |
|---|---|---|---|---|---|
| Ex- | Structure | | LCMS | Method, | |
| ample | (R) | Name | (M + H)⁺ | RT (min.) | ¹H NMR |
| 353 | 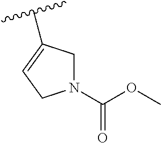 [SMILES: *C1=CCN(C(=O)OC)C1] | Methyl (R)-3-(4-amino-5-(3- (4-(2-(dimethylamino)ethoxy)- 2-methylthiazole-5- carboxamido)piperidin-1- yl)pyrrolo[2,1-f][1,2,4]triazin- 7-yl)-2,5-dihydro-1H-pyrrole- 1-carboxylate | 570.3 | E: 1.59 F: 0.98 | (400 MHz, DMSO-d₆) δ ppm 8.01 (br s, 1H), 7.85 (s, 1H), 7.62 (br d, J = 9.8 Hz, 1H), 6.85 - 6.77 (br s, 1H), 6.78-6.69 (m, 2H), 4.59-4.43 (m, 4H), 4.29 (br s, 2H), 4.12 (br s, 1H), 3.65 (d, J = 4.3 Hz, 3H), 3.10 (br s, 1H), 2.95 (br s, 1H), 2.80 (br s, 1H), 2.60 (s, 6H), 2.18 (s, 6H), 1.84 (br s, 3H), 1.47 (br s, 1H) |
| 354 | 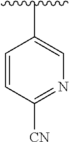 [SMILES: *c1ccc(C#N)nc1] | (R)-N-(1-(4-amino-7-(6- cyanopyridin-3-yl)pyrrolo[2,1- f][1,2,4]triazin-5-yl)piperidin- 3-yl)-4-(2- (dimethylamino)ethoxy)-2- methylthiazole-5-carboxamide | 547.2 | E: 1.65 F: 1.00 | (400 MHz, DMSO-d₆) δ ppm 9.44 (d, J = 2.0 Hz, 1H), 8.81 (dd, J = 2.3, 8.5 Hz, 1H), 8.32- 8.19 (m, 1H), 8.09 (d, J = 8.3 Hz, 1H), 7.96 (s, 1H), 7.65 (br d, J = 8.3 Hz, 1H), 7.42 (s, 1H), 7.02-6.88 (m, 1H), 4.50 (t, J = 5.4 Hz, 2H), 4.17 (br s, 1H), 3.17 (br d, J = 5.3 Hz, 4H), 3.02 (br s, 1H), 2.65-2.53 (m, 6H), 2.33-2.15 (m, 6H), 1.88 (br s, 3H), 1.60-1.42 (m, 1H) |
| 355 | 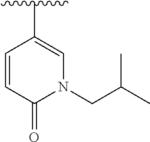 [SMILES: *c1ccc(=O)n(CC(C)C)c1] | (R)-N-(1-(4-amino-7-(1- isobutyl-6-oxo-1,6- dihydropyridin-3- yl)pyrrolo[2,1-f][ 1,2,4]triazin- 5-yl)piperidin-3-yl)-4-(2- (dimethylamino)ethoxy)-2- methylthiazole-5-carboxamide | 594.3 | E: 1.51 F: 0.99 | (400 MHz, DMSO-d₆) δ ppm 8.43 (d, J = 2.8 Hz, 1H), 8.03 (dd, J = 2.6, 9.6 Hz, 1H), 7.81 (s, 1H), 7.63 (s, 1H), 6.91 (s, 1H), 6.88 (m, 2H), 6.48 (d, J = 9.5 Hz, 1H), 4.54-4.44 (m, 2H), 4.13 (br s, 1H), 3.78 (d, J = 7.0 Hz, 2H), 3.17 (d, J = 5.3 Hz, 2H), 2.99 (br s, 2H), 2.82 (s, 1H), 2.60 (s, 4H), 2.29- 2.07 (m, 5H), 1.91 (s, 4H), 1.49 (s, 1H), 0.89 (d, J = 6.8 Hz, 6H) |
| 356 | 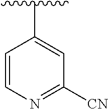 [SMILES: *c1ccnc(C#N)c1] | (R)-N-(1-(4-amino-7-(2- cyanopyridin-4-yl)pyrrolo[2,1- f][1,2,4]triazin-5-yl)piperidin- 3-yl)-4-(2- (dimethylamino)ethoxy)-2- methylthiazole-5-carboxamide | 547.3 | F: 1.001 | (400 MHz, DMSO-d₆) δ ppm 8.79-8.69 (m, 2H), 8.55-8.49 (m, 1H), 8.28 (br s, 1H), 8.01 (s, 1H), 7.69-7.57 (m, 1H), 7.53 (s, 1H), 6.96 (br s, 1H), 4.50 (t. J = 5.4 Hz, 2H), 4.17 (br s, 1H), 3.17 (br d, J = 5.3 Hz, 4H), 3.02 (br s, 1H), 2.65- 2.53 (m, 6H), 2.33-2.15 (m, 6H), 1.88 (br s, 3H), 1.60-1.42 (m, 1H) |

### Example 357

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-2-methyl-4-((1-methylpiperidin-4-yl)oxy)thiazole-5-carboxamide

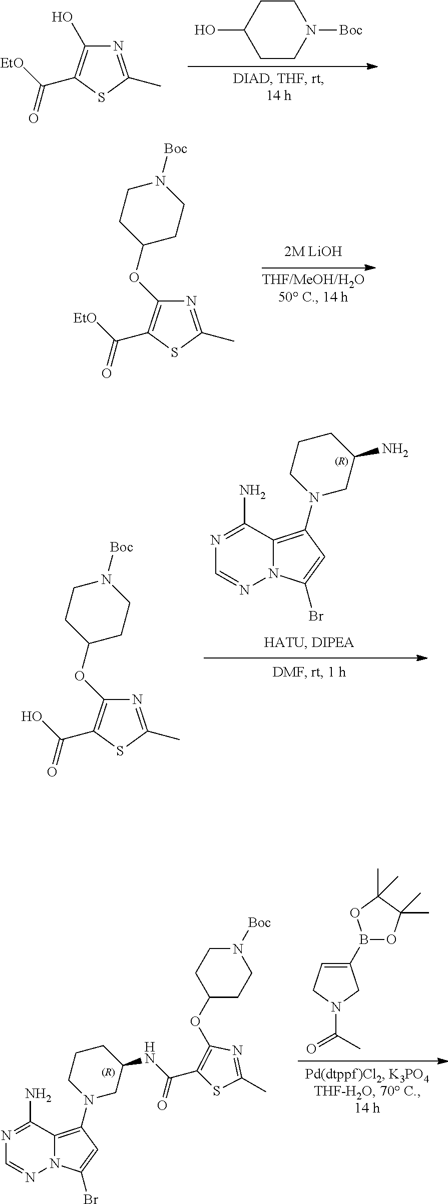

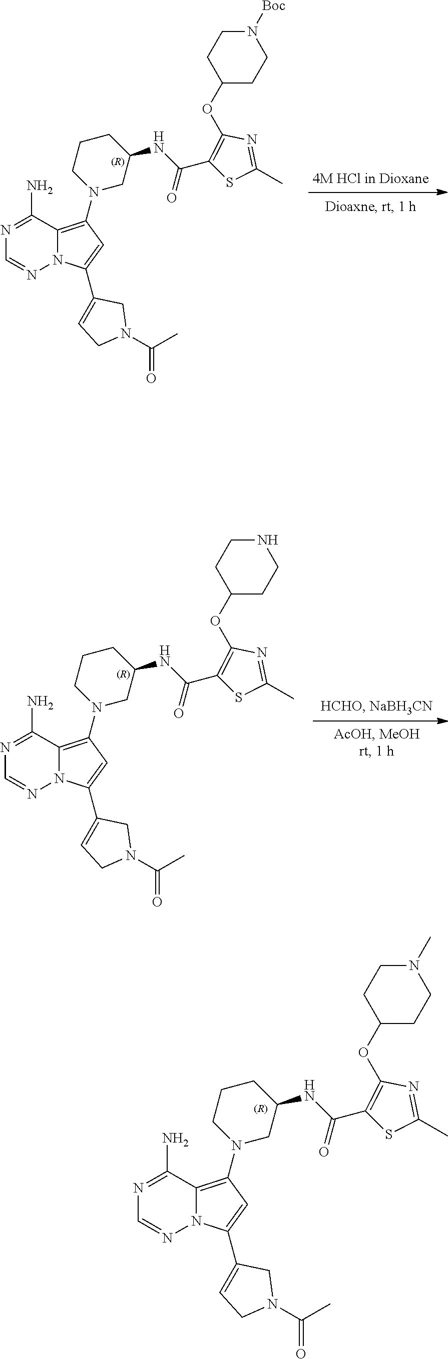

### Example 357A. Ethyl 4-(1-(tert-butoxycarbonyl)piperidin-4-yl)oxy)-2-methylthiazole-5-carboxylate

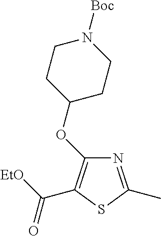 [SMILES: CCOC(=O)c1sc(C)nc1OC1CCN(C(=O)OC(C)(C)C)CC1]

To a stirred solution of ethyl 4-hydroxy-2-methylthiazole-5-carboxylate (1 g, 5.34 mmol) and tert-butyl 4-hydroxypiperidine-1-carboxylate (1.07 g, 5.34 mmol) in THF (10 mL), was added triphenylphosphine (1.82 g, 6.94 mmol). Reaction mixture was stirred at rt for 10 min and then DIAD (1.04 mL, 5.34 mmol) was added. Resulting mixture was allowed to stir at rt for 14 h followed by the addition of water (40 mL) and EtOAc (50 mL). Organic layer was separated, dried over Na₂SO₄, filtered and concentrated. The crude product was purified by silica gel chromatography (20% EtOAc in pet ether) which afforded Example 357A (1.2 g, 61% yield) as a gummy liquid. MS: [M+H]⁺=371.2; ¹H NMR (400 MHz, CDCl₃) δ ppm 5.20 (tt, J=3.4, 6.7 Hz, 1H), 4.40-4.26 (m, 2H), 3.67 (ddd, J=3.8, 8.7, 13.2 Hz, 2H), 3.56-3.45 (m, 2H), 2.63 (s, 3H), 2.06 (s, 1H), 1.98-1.81 (m, 2H), 1.49 (s, 9H), 1.40-1.26 (m, 3H).

### Example 357B. 4-((1-(tert-butoxycarbonyl) piperidin-4-yl) oxy)-2-methylthiazole-5-carboxylic acid, Li-salt

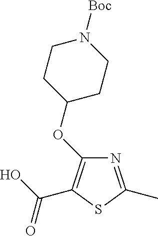 [SMILES: Cc1nc(OC2CCN(C(=O)OC(C)(C)C)CC2)c(C(=O)O)s1]

Example 357A (1.2 g, 3.24 mmol) was dissolved in THF (12 mL) and MeOH (2 mL). Lithium hydroxide monohydrate (2M 5 mL aq. solution, 9.72 mmol) was added. The reaction was heated at 50° C. for 14 h. Then, it was concentrated to dryness which afforded Example 357B (0.9 g, 81% Yield) as an off-white solid. MS: [M−H]⁺=341.2; ¹H NMR (400 MHz, DMSO-d₆) δ ppm 4.92 (td, J=4.0, 8.0 Hz, 1H), 3.75-3.64 (m, 2H), 3.32-3.08 (m, 2H), 2.46 (s, 3H), 1.87-1.72 (m, 2H), 1.68-1.49 (m, 6H), 1.41 (s, 9H).

### Example 357C. tert-butyl (R)-4-((5-((1-(4-amino-7-bromopyrrolo[2,1-f][1,2,4]triazin-5-yl)piperidin-3-yl)carbamoyl)-2-methylthiazol-4-yl)oxy)piperidine-1-carboxylate

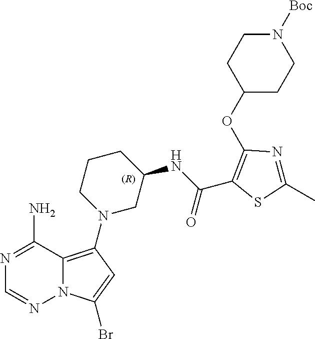

Example 357B (1.09 g, 2.88 mmol), (R)-5-(3-aminopiperidin-1-yl)-7-bromopyrrolo[2,1-f][1,2,4]triazin-4-amine (1 g, 3.1 mmol) and DIEA (1.5 mL, 1.11 mmol) were taken in DMF (10 mL). HATU (1.2 g, 3.16 mmol) was added and the reaction mixture was stirred at rt for 1 h. Reaction mixture was diluted with water (50 mL) and EtOAc (2×50 mL). Organic phase was separated, dried over Na₂SO₄, filtered and concentrated. The crude product was purified by silica-gel chromatography (5% MeOH in DCM) which afforded Example 357C (0.92 g, 52% Yield) as a yellow solid. MS: [M+H]⁺=637.2; ¹H NMR (400 MHz, DMSO-d₆) δ ppm 8.16 (d, J=8.5 Hz, 1H), 7.90-7.82 (m, 2H), 7.71-7.64 (m, 1H), 7.31-7.17 (m, 1H), 6.79-6.75 (m, 1H), 5.12 (td, J=4.0, 7.7 Hz, 1H), 4.13 (br s, 1H), 3.57 (br s, 2H), 3.50 (br s, 1H), 3.28-3.17 (m, 2H), 3.09 (br d, J=11.0 Hz, 1H), 2.91 (br s, 1H), 2.84-2.76 (m, 2H), 2.61 (s, 3H), 2.00-1.87 (m, 3H), 1.81 (br d, J=17.6 Hz, 4H), 1.66 (br d, J=7.5 Hz, 3H), 1.52 (br s, 1H), 1.42 (s, 9H), 1.30-1.12 (m, 2H).

### Example 357D. tert-butyl(R)-4-((5-((1-(7-(1-acetyl-2,5-dihydro-1H-pyrrol-3-yl)-4-aminopyrrolo[2,1-f][1,2,4]triazin-5-yl)piperidin-3-yl)carbamoyl)-2-methylthiazol-4-yl)oxy)piperidine-1-carboxylate

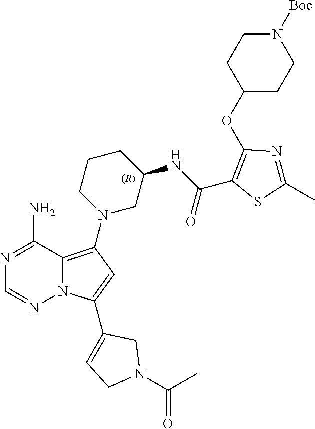

Example 357C (0.3 g, 0.472 mmol), 1-(3-(4,4,5,5-tetramethyl-1,3,2-dioxaborolan-2-yl)-2,5-dihydro-1H-pyrrol-1-yl)ethan-1-one (0.17 g, 0.708 mmol) and K₃PO₄ (0.2 g, 0.944 mmol), were taken in THF (4 mL)-water (0.5 mL). The mixture was degassed with N₂ and then PdCl₂(dtppf) (22 mg, 0.033 mmol) was added. It was heated at 70° C. for 14 h. After cooling, reaction mixture was diluted with water (25 mL) and EtOAc (2×25 mL). Organic phase was separated, dried over Na₂SO₄, filtered and concentrated. The crude product was purified by silica-gel chromatography (5% MeOH in DCM) which afforded Example 357D (0.25 g, 80% yield) as a yellow solid. MS: [M+H]⁺=666.4.

### Example 357E. (R)—N-(1-(7-(1-acetyl-2,5-dihydro-1H-pyrrol-3-yl)-4-aminopyrrolo[2,1-f][1,2,4]triazin-5-yl)piperidin-3-yl)-2-methyl-4-(piperidin-4-yloxy)thiazole-5-carboxamide

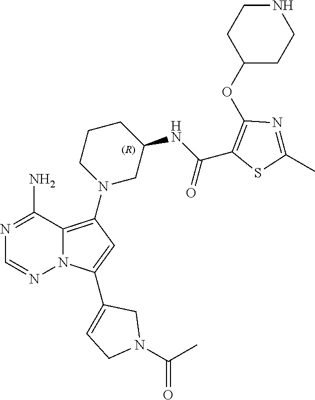

To a solution of Example 357D (40 mg, 0.060 mmol) in 1,4-dioxane (2 mL), was added HCl (4M in dioxane, 2 mL, 20.0 mmol). The reaction mixture was stirred at rt for 1 h. Solvent was removed under reduced pressure which afforded Example 357E (30 mg, 85% yield) as a yellow solid. LC-MS Method E: RT=1.07 min, [M+H]⁺=566.2; LC-MS Method F: RT=0.84 min, [M+H]J=566.2; ¹H NMR (400 MHz, DMSO-d₆) δ ppm 7.86 (d, J=1.8 Hz, 1H), 7.34 (br s, 1H), 6.79-6.68 (m, 2H), 5.06-4.93 (m, 1H), 4.68 (br s, 1H), 4.54-4.41 (m, 2H), 4.26 (br s, 1H), 4.15 (br s, 1H), 3.15-3.10 (m, 1H), 3.00-2.70 (m, 5H), 2.64-2.57 (m, 4H), 2.05-1.94 (m, 6H), 1.92-1.79 (m, 4H), 1.66-1.40 (m, 3H).

### Example 357

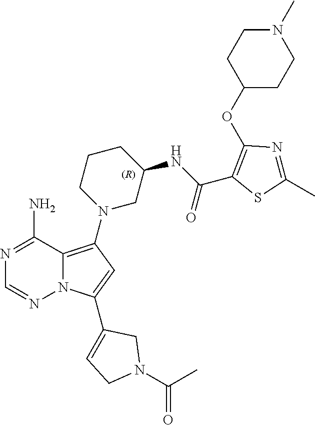

To a stirred solution of Example 357E (50 mg, 0.083 mmol) and acetic acid (0.475 μl, 8.30 μmol) in methanol (2 mL), was added formaldehyde (38% aq. solution 0.030 mL, 0.415 mmol). The mixture was stirred at rt for 10 min and then sodium cyanoborohydride (10.4 mg, 0.166 mmol) was added. The mixture was stirred for another 1 h, concentrated to dryness and residue was diluted with water (15 mL) and ethyl acetate (30 mL). Organic layer was separated, dried over Na₂SO₄, filtered and concentrated. Resultant residue was purified by preparative HPLC to afford Example 357. LC-MS Method E: RT=1.178 min, [M+H]⁺=580.3; LC-MS Method F: RT=0.848 min, [M+H]⁺=580.3; ¹H NMR (400 MHz, DMSO-d₆) δ ppm 8.02 (br s, 1H), 7.86 (d, J=1.5 Hz, 1H), 7.28 (br s, 1H), 6.80-6.73 (m, 1H), 6.71 (d, J=11.8 Hz, 1H), 5.07-4.99 (m, 1H), 4.68 (br d, J=2.0 Hz, 1H), 4.52-4.42 (m, 2H), 4.26 (br d, J=2.0 Hz, 1H), 4.16 (br s, 1H), 3.19-2.92 (m, 3H), 2.81 (br s, 2H), 2.60 (s, 4H), 2.56-2.52 (m, 2H), 2.42-2.32 (m, 2H), 2.19 (br d, J=0.8 Hz, 2H), 2.07-1.94 (m, 6H), 1.93-1.71 (m, 6H), 1.54 (br s, 1H).

The following examples in Table 22 were prepared using the same procedure as shown in Example 357. 357C was coupled with the appropriate boronic acids or boronate esters, followed by Boc removal and reductive amination.

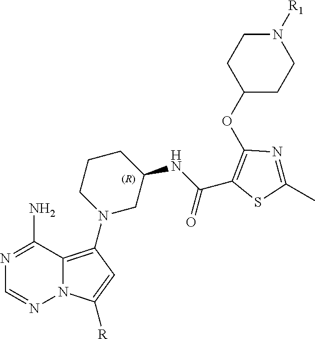

TABLE 22

| | | | | | HPLC | |
|---|---|---|---|---|---|---|
| | | | | LCMS | Method, | |
| Example | R | R1 | Name | (M + H)⁺ | RT (min.) | ¹H NMR |
| 358 | 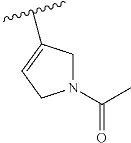 [SMILES: *C1=CCN(C(C)=O)C1] | Isopropyl | (R)-N-(1-(7-(1-acetyl-2,5- dihydro-1H-pyrrol-3-yl)-4- aminopyrrolo[2,1- f][1,2,4]triazin-5- yl)piperidin-3-yl)-4-((1- isopropylpiperidin-4- yl)oxy)-2-methylthiazole-5- carboxamide | 608.3 | E: 1.212 F: 0.898 | (400 MHz, DMSO-d₆) δ ppm 8.05 (br s, 1H), 7.87 (d, J = 1.0 Hz, 1H), 7.27-7.26 (m, 1H), 7.38-7.20 (m, 1H), 6.82-6.75 (m, 1H), 6.71 (d, J = 9.8 Hz, 1H), 5.05 (br s, 1H), 4.69 (br s, 1H), 4.48 (br d, J = 6.3 Hz, 2H), 4.27 (br s, 1H), 4.16 (br s, 1H), 3.60 (br s, 1H), 3.17 (br d, J = 5.0 Hz. 1H), 3.01-2.69 (m, 4H), 2.61 (s, 3H), 2.54 (s, 1H), 2.03 (d, J = 16.3 Hz, 4H), 1.81 (br s, 6H), 1.55 (br s, 2H), 0.98 (br s, 6H) |
| 359 | 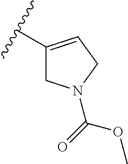 [SMILES: *C1=CCN(C(=O)OC)C1] | H | Methyl (R)-3-(4-amino-5- (3-(2-methyl-4-(piperidin-4- yloxy)thiazole-5- carboxamido)piperidin-1- yl)pyrrolo[2,1- f][1,2,4]triazin-7-yl)-2.5- dihydro-1H-pyrrole-1- carboxylate | 582.2 | E: 1.081 F: 0.962 | (400 MHz, DMSO-d₆) δ ppm 8.01 (br s, 1H), 7.85 (s, 1H), 7.31 (s, 1H), 6.78-6.65 (m, 3H), 5.06-4.98 (m, 1H), 4.58-4.47 (m, 2H), 4.29 (dt, J = 1.4, 4.6 Hz, 2H), 4.19- 4.09 (m, 1H), 3.67-3.60 (m, 4H), 3.16 (br d, J = 0.8 Hz, 1H), 3.01-2.93 (m, 3H), 2.69 (br s, 1H), 2.60 (s, 4H), 2.05-1.96 (m, 2H), 1.89-1.75 (m, 3H), 1,63 (dt, J = 1.9, 3.6 Hz, 3H) |
| 360 | 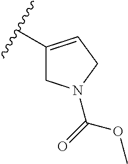 [SMILES: *C1=CCN(C(=O)OC)C1] | Me | Methyl (R)-3-(4-amino-5- (3-(2-methyl-4-((1- methylpiperidin-4- yl)oxy)thiazole-5- carboxamido)piperidin-1- yl)pyrrolo[2,1- f][1,2,4]triazin-7-yl)-2,5- dihydro-1H-pyrrole-1- carboxylate | 596.2 | E: 1.351 F: 0.979 | (400 MHz, DMSO-d₆) δ ppm 8.01 (br s, 1H), 7.85 (s, 1H), 7.24 (s, 1H), 6.76-6.66 (m, 3H), 5,07-4.94 (m, 1H), 4.58-4.47 (m, 1H), 4,34-4.24 (m, 2H), 4.14 (br s, 1H), 3.90 (s, 1H), 3.65 (d, J = 4.3 Hz, 3H), 3.16 (s, 1H), 2.82 (s, 2H), 2.60 (s, 4H), 2.46-2.41 (m, 1H), 2.31-2.23 (m, 1H), 2.15 (br d, J = 1.5 Hz, 3H), 2.02-1.91 (m, 3H), 1.86-1.70 (m, 5H), 1.52 (s, 1H) |
| 361 | 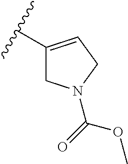 [SMILES: *C1=CCN(C(=O)OC)C1] | Isopropyl | Methyl (R)-3-(4-amino-5- (3-(4-((1- isopropylpiperidin-4- yl)oxy)-2-methylthiazole-5- carboxamido)piperidin-1- yl)pyrrolo[2,1- f][1,2,4]triazin-7-yl)-2,5- dihydro-1H-pyrrole-1- carboxylate | 624.3 | E: 1.403 F: 1.040 | (400 MHz, DMSO-d₆) δ ppm 8.01 (br s, 1H), 7.85 (s, 1H), 7.26 (s, 1H), 6.77-6.64 (m, 2H), 5.02 (br s, 1H), 4.58-4.45 (m. 2H), 4.35-4.24 (m, 2H), 4.21-4.05 (m, 1H), 3.65 (d, J = 4.0 Hz, 3H), 3.19-3.11 (m, 2H), 2.93 (br s, 2H), 2.81 (br s, 3H), 2.60 (s, 4H), 2.04-1.73 (m, 7H), 1.52 (s, 1H), 0.92 (br s, 6H) |
| 362 | 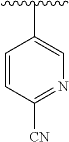 [SMILES: *c1ccc(C#N)nc1] | Me | (R)-N-(1-(4-amino-7-(6- cyanopyridin-3- yl)pyrrolo[2,1- f][1,2,4]triazin-5- yl)piperidin-3-yl)-2-methyl- 4-((1-methylpiperidin-4- yl)oxy)thiazole-5- carboxamide | 573.3 | E: 1.450 F: 1.011 | (400 MHz, DMSO-d₆) δ ppm 9.43 (d, J = 2.0 Hz, 1H), 8.80 (dd, J = 2.3, 8.3 Hz, 1H), 8.20 (s, 1H), 8.09 (d, J = 8.5 Hz, 1H), 7.95 (s, 1H), 7.41 (s, 1H), 7.29 (br s, 1H), 6.86 (s, 1H), 5.02 (br s, 1H), 4.17 (br s, 1H), 3.23-3.18 (m, 1H), 3.01 (br s, 1H), 2.85 (br s, 2H), 2.60 (s, 3H), 2.32-2.22, (m, 2H), 2.14 (br s, 4H), 2.03-1.73 (m, 8H), 1.56 (br s, 1H) |
| 363 | 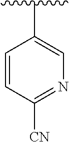 [SMILES: *c1ccc(C#N)nc1] | Isopropyl | (R)-N-(1-(4-amino-7-(6- cyanopyridin-3- yl)pyrrolo[2,1- f][1,2,4]triazin-5- yl)piperidin-3-yl)-4-((1- isopropylpiperidin-4- yl)oxy)-2-methylthiazole-5- carboxamide | 601.3 | E: 1.527 F: 1.074 | (400 MHz, DMSO-d₆) δ ppm 9.42 (s, 1H), 8.80 (dd, J = 2.0, 8.5 Hz, 1H), 8.09 (d, J = 8.5 Hz, 2H), 7.95 (s, 1H), 7.40 (s, 2H), 6.87 (br s, 1H), 5.05-4.93 (m, 1H), 4.23-4.10 (m, 1H), 3.23-3.17 (m, 1H), 3.05-2.72 (m, 2H), 2.60 (s, 6H), 2.43 (br s, 1H), 2.03-1.67 (m, 6H), 1.56 (br s, 1H), 0.89 (br s, 6H) |

### Example 364

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-3-(2-(dimethylamino)ethoxy)-1H-indole-2-carboxamide

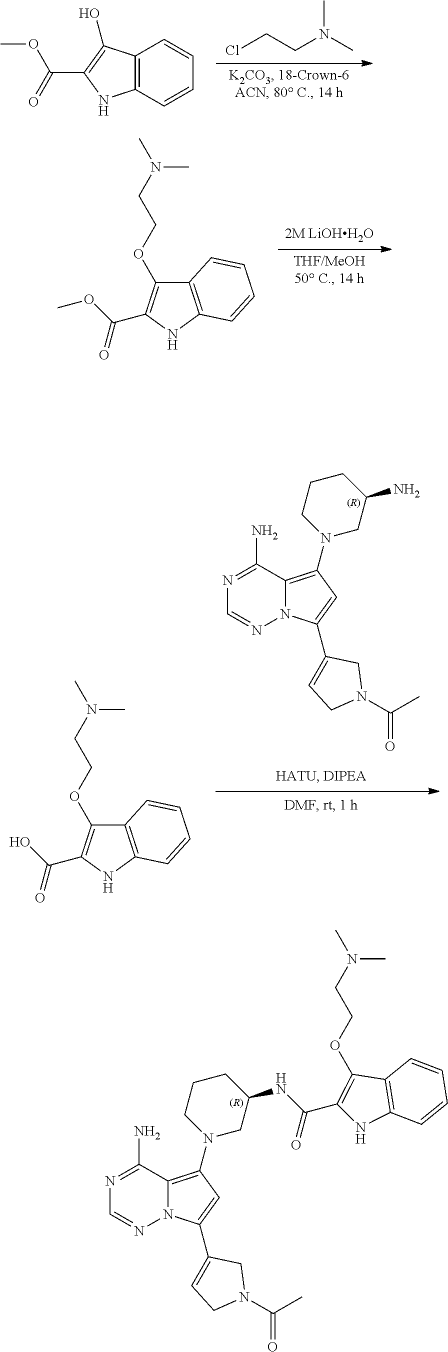

### Example 364A. Methyl 3-(2-(dimethylamino)ethoxy)-1H-indole-2-carboxylate

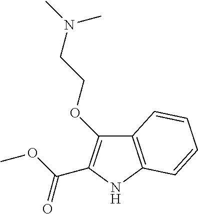 [SMILES: COC(=O)c1[nH]c2ccccc2c1OCCN(C)C]

To a stirred solution of methyl 3-hydroxy-1H-indole-2-carboxylate (0.5 g, 2.62 mmol), 2-chloro-N,N-dimethylethan-1-amine (0.33 g, 3.14 mmol) and K₂CO₃ (0.72 g, 5.23 mmol) in THF (5 mL), was added 18-crown-6 (69 mg, 0.26 mmol). Reaction mixture was heated at 80° C. for 14 h. After cooling, water (25 mL) and EtOAc (2×30 mL) were added. Organic phase was separated, dried over Na₂SO₄, filtered and concentrated which afforded Example 364A (0.6 g, 70% yield) as a yellow solid. MS: [M+H]⁺=263.0.

### Example 364B. 3-(2-(dimethylamino)ethoxy)-1H-indole-2-carboxylic acid, Lithium salt

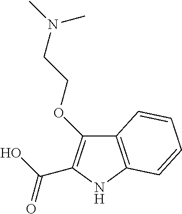 [SMILES: CN(C)CCOc1c(C(=O)O)[nH]c2ccccc12]

Lithium hydroxide monohydrate (2M aq. soln. 2 mL, 10.45 mol) was added to a solution of Example 364A (0.45 g, 1.71 mmol) in THF (8 mL)/MeOH (1 mL). Reaction mixture was heated at 50° C. for 14 h. It was concentrated to dryness which afforded Example 364B (0.7 g, 87% Yield) as an off-white solid. MS: [M+H]⁺=231.2.

### Example 364

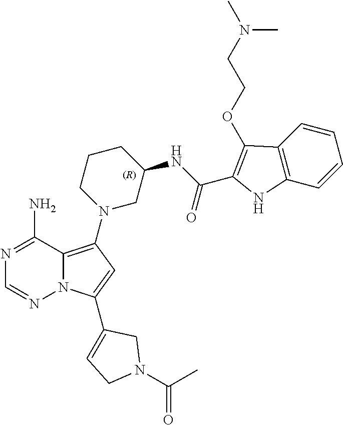

Example 364B (50 mg, 0.132 mmol), 3-(2-(dimethylamino)ethoxy)-1H-indole-2-carboxylic acid lithium salt (33 mg, 0.132 mmol) and DIEA (0.07 mL, 0.397 mmol) were suspended in anhydrous DMF (1 mL). HATU (0.59 mg, 0.132 mmol) was added and the reaction mixture was stirred at rt for 1 h. Diluted with water (15 mL) and EtOAc (2×15 mL) and organic phase was separated, dried over Na₂SO₄, filtered and concentrated. The crude product was purified by preparative HPLC to afford the Example 364 (26 mg, 89% yield) as a yellow solid. LC-MS Method E: RT=1.48 min, [M+H]⁺=572.3; LC-MS Method F: RT=0.93 min, [M+H]⁺=572.3; ¹H NMR (400 MHz, DMSO-d₆) δ ppm 11.16 (s, 1H), 8.15-8.06 (m, 1H), 8.02 (br s, 1H), 7.88 (s, 1H), 7.74 (d, J=8.5 Hz, 1H), 7.40-7.32 (m, 1H), 7.22-7.16 (m, 1H) 7.03 (d, J=7.5 Hz, 1H), 6.89 (br s, 1H), 6.83-6.68 (m, 2H), 4.69 (br d, J=3.3 Hz, 1H), 4.53-4.40 (m, 4H), 4.28-4.20 (m, 2H), 3.04 (br s, 2H), 2.72 (br s, 1H), 2.59 (br dd, J=2.8, 6.8 Hz, 2H), 2.21 (br s, 6H), 2.03 (d, J=15.5 Hz, 4H), 1.87 (br d, J=2.5 Hz, 2H), 1.50 (br s, 1H).

The following examples in Table 23 were prepared using the same procedure as shown in Example 364. Example 364B was coupled with respective amines which were synthesized as described in Intermediate 31.

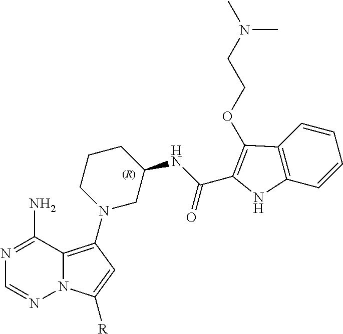

TABLE-12

| | | | | HPLC | |
|---|---|---|---|---|---|
| | | | LCMS | Method, | |
| Example | R | Name | (M + H)⁺ | RT (min.) | ¹H NMR |
| 365 | 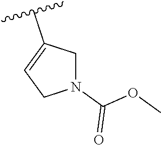 [SMILES: *C1=CCN(C(=O)OC)C1] | Methyl (R)-3- (4-amino-5-(3- (3-(2- (dimethylamino) ethoxy)-1H- indole-2- carboxamido) piperidin-1- yl)pyrrolo [2,1- f][1,2,4]triazin- 7-yl)-2,5- dihydro-1H- pyrrole-1- carboxylate | 588.3 | E: 1.77 F: 1.08 | (400 MHz, DMSO-d₆) δ ppm 11.30 (s, 1H), 9.86 (br s, 1H), 8.02 (br s, 1H), 7.91 (s, 1H), 7.74 (br d, J = 7.8 Hz. 2H), 7.42 (d, J = 8.3 Hz, 1H), 7.29-7.23 (m, 1H), 7.23-6.93 (m, 1H), 6.80-6.70 (m, 2H), 4.62-4.50 (m, 4H), 4.33-4.23 (m, 3H), 3.65 (d, J = 3.8 Hz, 3H), 3.54- 3.51 (m, 2H), 3.30-3.27 (m, 1H), 3.07-3.02 (m, 1H), 2.94 (br s, 6H), 2.73 (br s, 2H), 1.87 (br s, 3H), 1.57 (br s, 1H) |
| 366 | 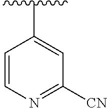 [SMILES: *c1ccnc(C#N)c1] | (R)-N-(1-(4- amino-7-(2- cyanopyridin-4- yl)pyrrolo[2,1- f][1,2,4]triazin-5- yl)piperidin-3- yl)-3-(2- (dimethylamino )ethoxy)-1H- indole-2- carboxamide | 565.3 | E: 1.843 F: 1.122 | (400 MHz, DMSO-d₆) δ ppm 11.31 (s, 1H), 9.86 (br s, 1H), 8.79-8.68 (m, 2H), 8.53 (dd, J = 1.8, 5.3 Hz, 1H), 8.32 (br s, 1H), 8.04 (s, 1H), 7.75 (d, J = 8.0 Hz, 2H), 7.54 (s, 1H), 7.43 (d, J = 8.3 Hz, 1H), 7.32-6.92 (m, 2H), 4.56 (br t, J = 5.0 Hz, 2H), 4.27 (br s, 1H), 3.54 (br d, J = 4.3 Hz, 2H), 3.08 (br m, 2H), 2.95 (br dd, J = 4.8, 7.0 Hz, 6H), 2.79 (br s, 2H), 2.00 (br s, 1H), 1.91 (br s, 2H), 1.60 (br s, 1H) |
| 367 | 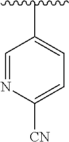 [SMILES: *c1ccc(C#N)nc1] | (R)-N-(1-(4- amino-7-(6- cyanopyridin-3- yl)pyrrolo[2,1- f][1,2,4]triazin-5- yl)piperidin-3-yl)- 3-(2- (dimethylamino) ethoxy)-1H- indole-2- carboxamide | 565.3 | E: 1.841 F: 1.113 | (400 MHz, DMSO-d₆) δ ppm 11.19 (s, 1H), 9.49-9.38 (m, 1H), 8.81 (dd, J = 2.3, 8.3 Hz, 1H), 8.09 (d, J = 8.3 Hz, 1H), 7.96 (s, 1H), 7.91-7.85 (m, 1H), 7.74 (d, J = 8.0 Hz, 1H), 7.42 (s, 1H), 7.40-7.32 (m, 1H), 7.28 (d, J = 8.8 Hz, 1H), 7.22-7.12 (m, 1H), 7.06-6.90 (m, 1H), 6.81 (d, J = 10.8 Hz, 1H), 4.55-4.36 (m, 2H), 4.32-3.97 (m, 1H), 3.81-3.62 (m, 2H), 3.12-3.02 (m, 1H), 2.89-2.70 (m, 2H), 2.43-2.09 (m, 6H), 2.03-1.79 (m, 4H), 1.52 (br s, 1H) |
| 368 | 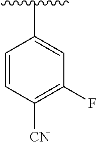 [SMILES: *c1ccc(C#N)c(F)c1] | (R)-N-(1-(4- amino-7-(4- cyano-3- fluorophenyl) pyrrolo[2,1- f][1,2,4]triazin-5- yl)piperidin-3- yl)-3-(2- (dimethylamino) ethoxy)-1H- indole-2- carboxamide | 582.3 | E: 2.087 F: 1.296 | (400 MHz, DMSO-d₆) δ ppm 11.19 (s. 1H), 8.37 (dd, J = 1.3, 12.0 Hz, 1H), 8.23 (dd, J = 1.4, 8.4 Hz, 3H), 7.99-7.90 (m, 2H), 7.74 (d, J = 8.3 Hz, 1H), 7.42-7.33 (m, 2H), 7.20 (t, J = 7.5 Hz, 1H), 7.03 (br t, J = 7.5 Hz, 2H), 4.55-4.42 (m, 2H), 4.28 (br s, 1H), 3.13-3.00 (m, 1H), 2.76 (br s, 4H), 2.30 (br s, 6H), 2.00 (br s, 1H), 1.89 (br s, 2H), 1.55 (br s, 1H) |
| 369 | 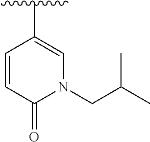 [SMILES: *c1ccc(=O)n(CC(C)C)c1] | (R)-N-(1-(4- amino-7-(1- isobutyl-6-oxo- 1,6- dihydropyridin-3- yl)pyrrolo[2,1- f][1,2,4]triazin-5- yl)piperidin-3- yl)-3-(2- (dimethylamino) ethoxy)-1H- indole-2- carboxamide | 612.3 | E: 1.742 F: 1.102 | (400 MHz, DMSO-d₆) δ ppm 11.17 (s, 1H), 8.44 (d, J = 1.8 Hz, 1H), 8.15-8.08 (m, 1H), 8.04 (br dd, J = 2.3, 9.5 Hz, 1H), 7.82 (s, 2H), 7.74 (br d, J = 8.3 Hz, 1H), 7.64-7.41 (m, 2H), 7.37 (br d, J = 8.3 Hz, 1H), 7.19 (br t, J = 7.4 Hz, 1H), 7.02 (br t,) = 7.5 Hz, 1H), 6.92 (s, 1H), 6.49 (d, J = 9.5 Hz, 1H), 4.54-4.41 (m, 3H), 4.23 (br s, 2H), 3.78 (br d, J = 7.3 Hz, 2H), 3.08-3.01 (m, 1H), 2.81-2.70 (m, 2H), 2.62 (br d, J = 1.8 Hz, 1H), 2.24 (br s, 6H), 2.12-2.04 (m, 1H), 2.02-1.95 (m, 1H), 1.88 (br s, 2H), 1.57-1.46 (m, 1H), 0.89 (br d, J = 6.8 Hz, 6H) |

### Example 370

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-cyclopropyl-3-(2-(dimethylamino)ethoxy)thiophene-2-carboxamide

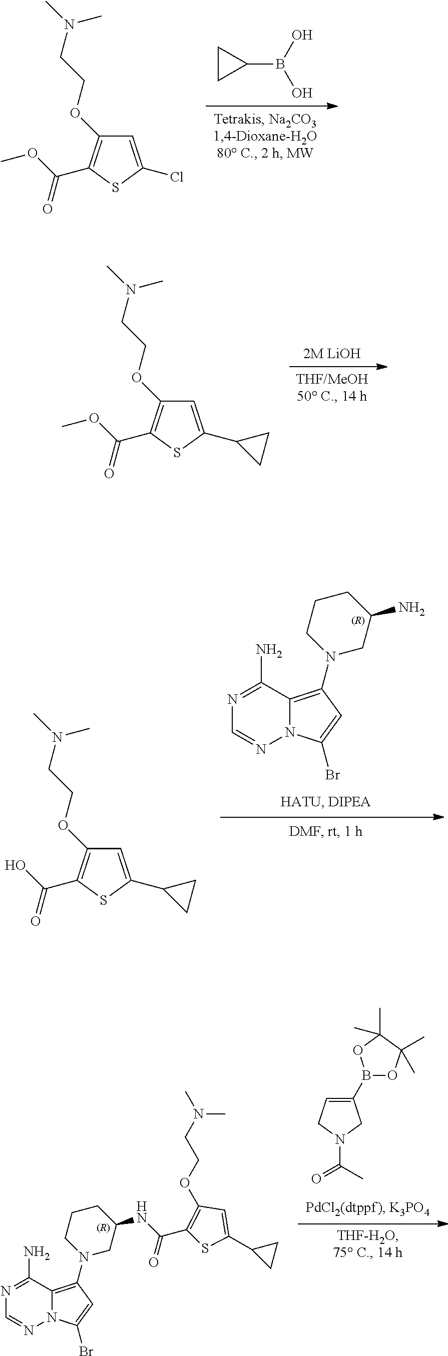

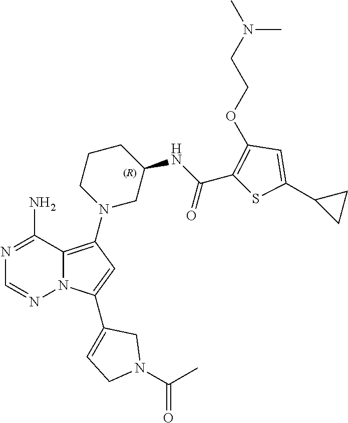

### Example 370A. Methyl 5-cyclopropyl-3-(2-(dimethylamino)ethoxy)thiophene-2-carboxylate

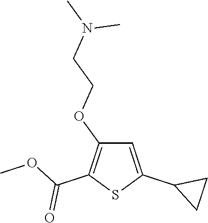 [SMILES: COC(=O)c1sc(C2CC2)cc1OCCN(C)C]

In a 30 mL microwave vial, methyl 5-chloro-3-(2-(dimethylamino)ethoxy)thiophene-2-carboxylate (0.36 g, 1.365 mmol), Na₂CO₃ (0.289 g, 2.73 mmol) and cyclopropylboronic acid (0.586 g, 6.83 mmol) were taken in 1,4-dioxane (5 mL)-Water (0.5 mL). The mixture was degassed with N₂ and Pd(PPh₃)₄ (0.095 g, 0.082 mmol) was added and the mixture was heated at 80° C. under microwave for 2 h. After cooling, water (50 mL) and ethyl acetate (2×50 mL) were added. Organic layer was separated, dried over Na₂SO₄, filtered and concentrated. The crude product was purified by silica-gel chromatography (5% MeOH in DCM) which afforded Example 370A (0.25 g, 69% yield) as a yellow gummy solid. MS: [M+H]⁺=270.2; ¹H NMR (400 MHz, CDCl₃) δ ppm 6.57 (s, 1H), 4.23-4.19 (m, 2H), 3.90-3.78 (m, 4H), 2.84-2.75 (m, 3H), 2.42-2.35 (m, 8H), 2.10-2.01 (m, 1H), 1.16-1.03 (m, 2H), 0.86-0.75 (m, 2H).

### Example 370B. 5-cyclopropyl-3-(2-(dimethylamino)ethoxy)thiophene-2-carboxylic acid

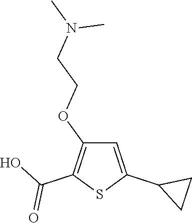 [SMILES: CN(C)CCOc1cc(C2CC2)sc1C(=O)O]

Analogous to the procedure for the preparation of Example 276B, reaction of Example 370A (0.4 g, 2.1 mmol) and Lithium hydroxide monohydrate (0.071 g, 2.97 mmol) afforded Example 370B (0.3 g, 80% yield) as an off-white solid. MS: [M+H]⁺=256.1; ¹H NMR (400 MHz, DMSO-d₆) δ ppm 6.75 (s, 1H), 4.31-4.18 (m, 2H), 2.90 (t, J=5.5 Hz, 2H), 2.08-2.01 (m, 1H), 1.04-0.95 (m, 2H), 0.71-0.66 (m, 2H).

### Example 370C. (R)—N-(1-(4-amino-7-bromopyrrolo[2,1-f][1,2,4]triazin-5-yl)piperidin-3-yl)-5-cyclopropyl-3-(2-(dimethylamino)ethoxy)thiophene-2-carboxamide

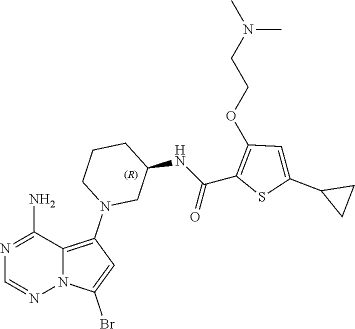

Analogous to the procedure described in Example 276C, reaction of (R)-5-(3-aminopiperidin-1-yl)-7-bromopyrrolo[2,1-f][1,2,4]triazin-4-amine (0.35 g, 1.01 mmol), Example 370B (0.386 g, 1.51 mmol) afforded Example 370C (0.30 g, 69% yield) as a yellow solid. MS: [M+H]⁺=550.2.

### Example 370

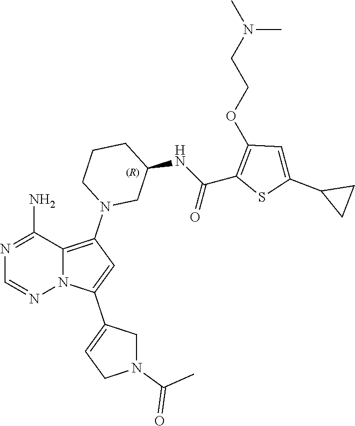

Analogous to the procedure described for Example 276, reaction of Example 370C (40 mg, 0.073 mmol), 1-(3-(4,4,5,5-tetramethyl-1,3,2-dioxaborolan-2-yl)-2,5-dihydro-1H-pyrrol-1-yl)ethan-1-one (25.9 mg, 0.109 mmol) afforded the Example 370 (11 mg, 30% yield). LCMS Method G: RT=1.48 min, [M+H]⁺=572.3; LCMS Method H; RT=0.93 min, [M+H]⁺=572.3.

The following Examples, 371-375 in Table 24 were prepared using the same procedure as shown in Example 276. Example 370C was coupled with the appropriate boronic acids or boronate esters.

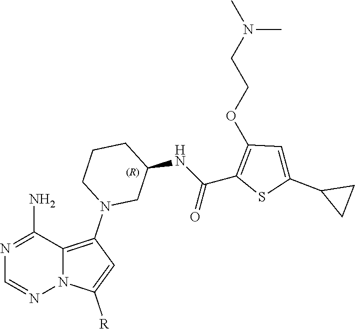

TABLE-24

| | | | | HPLC | |
|---|---|---|---|---|---|
| | | | LCMS | Method, | |
| Example | R | Name | (M + H)⁺ | RT (min.) | ¹H NMR |
| 371 | 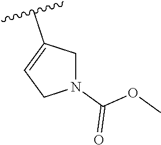 [SMILES: *C1=CCN(C(=O)OC)C1] | Methyl (R)-3-(4- amino-5-(3-(5- cyclopropyl-3-(2- (dimethylamino )ethoxy) thiophene-2- carboxamido) piperidin-1- yl)pyrrolo[2,1- f][1,2,4]triazin-7- yl)-2,5-dihydro- 1H-pyrrole-1- carboxylate | 595.4 | G: 4.41 K: 5.56 | (400 MHz, DMSO-d₆) δ ppm 8.02 (br s, 1H), 7.85 (s, 1H), 7.57 (br s, 1H), 7.35 (s, 1H), 6.90 (s, 1H), 6.73 (br s, 2H), 4.53 (br d, J = 10.6 Hz, 2H), 4.29 (br s, 4H), 4.13 (br s, 1H), 3.66 (d, J = 4.1 Hz, 3H), 3.27- 3.05 (m, 2H), 2.96 (br s, 2H), 2.48-2.30 (m, 4H), 2.26 (br s, 2H), 2.20-2.01 (m, 2H), 1.91 (br s, 1H), 1.83 (br s, 3H), 1.46 (br s, 1H), 1.09-1.02 (m, 2H), 0.76- 0.70 (m, 2H) |
| 372 | 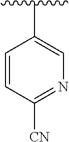 [SMILES: *c1ccc(C#N)nc1] | (R)-N-(1-(4- amino-7-(6- cyanopyridin-3- yl)pyrrolo[2,1- f][1,2,4]triazin- 5-yl)piperidin-3- yl)-5-cyclopropyl- 3-(2- (dimethylamino) ethoxy) thiophene-2- carboxamide | 572.2 | G: 4.62 K: 6.29 | (400 MHz, DMSO-d₆) δ ppm 9.43 (s, 1H), 8.81 (br d, J = 8.4 Hz, 1H), 8.20 (br s, 1H), 8.09 (d, J = 8.5 Hz, 1H), 7.95 (s, 1H), 7.67 (br d, J = 7.0 Hz, 1H), 7.41 (s, 1H), 6.94 (br s, 1H), 6.89 (s, 1H), 4.23 (br t, J = 4.9 Hz, 2H), 4.16 (br s, 1H), 3.01 (br s, 2H), 2.80 (br s, 1H), 2.65-2.54 (m, 3H), 2.25-2.07 (m, 6H), 1.85 (br d, J = 11.4 Hz, 4H), 1.46 (br s, 1H), 1.09-1.02 (m, 2H), 0.76-0.70 (m, 2H) |
| 373 | 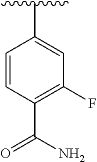 [SMILES: *c1ccc(C(N)=O)c(F)c1] | (R)-N-(1-(4-amino- 7-(4- carbamoyl-3- fluorophenyl) pyrrolo[2,1- f][1,2,4]triazin-5- yl)piperidin-3- yl)-5-cyclopropyl- 3-(2- (dimethylamino) ethoxy) thiophene-2- carboxamide | 607.3 | G: 4.79 K: 5.03 | (400 MHz, DMSO-d₆) δ ppm 8.12 (br d, J = 13.3 Hz, 1H), 8.03 (br s, 1H), 8.01 (br d, J = 8.4 Hz, 1H), 7.91 (s, 1H), 7.73 (br t. J = 7.9 Hz, 1H), 7.68-7.57 (m, 3H), 7.24 (s, 1H), 6.89 (s, 1H), 4.23 (br t, J = 5.2 Hz, 2H), 4.15 (br s, 1H), 3.01 (br s, 1H), 2.80 (br s, 2H), 2.65-2.54 (m, 3H), 2.17 (s, 6H), 2.12-2.07 (m, 1H), 1.84 (br s, 3H), 1.47 (br s, 1H), 1.08-1.00 (m, 2H), 0.76-0.69 (m, 2H) |
| 374 | 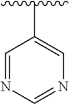 [SMILES: *c1cncnc1] | (R)-N-(1-(4- amino-7- (pyrimidin-5- yl)pyrrolo[2,1- f][1,2,4]triazin- 5-yl)piperidin-3- yl)-5-cyclopropyl- 3-(2- (dimethylamino) ethoxy) thiophene-2- carboxamide | 548.3 | E: 1.56 F: 0.96 | (400 MHz, DMSO-d₆) δ ppm 9.47 (d, J = 2.8 Hz, 2H), 9.09 (s, 1H), 8.14 (br s, 1H), 7.92 (s, 1H), 7.67- 7.57 (m, 1H), 7.31 (s, 1H), 6.90 (d, J = 2.4 Hz, 2H), 4.23 (brt, J = 5.2 Hz, 2H), 4.15 (br s, 1H), 3.01 (br s, 1H), 2.80 (br s, 2H), 2.65-2.54 (m, 3H), 2.17 (s, 6H), 2.12-2.07 (m, 1H), 1.84 (br s, 3H), 1.47 (br s, 1H), 1.08-1.00 (m, 2H), 0.76-0.69 (m, 2H) |
| 375 | 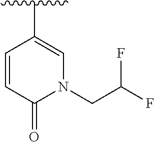 [SMILES: *c1ccc(=O)n(CC(F)F)c1] | (R)-N-(1-(4- amino-7-(1-(2,2- difluoroethyl)-6- oxo-1,6- dihydropyridin-3- yl)pyrrolo[2, 1-f] [1,2,4]triazin-5- yl)piperidin-3-yl)- 5- cyclopropyl-3-(2- (dimethylamino) ethoxy)thiophene- 2-carboxamide | 627.2 | E: 1.69 F: 1.06 | (400 MHz, DMSO-d₆) δ ppm 9.68 (s, 1H), 8.50 (s, 1H), 8.10 (dd, J = 2.7, 10.0 Hz, 1H), 7.86 (s, 1H), 7.13 (s, 1H), 6.91 (d, J = 7.8 Hz, 2H), 6.59 (d. J = 9.8 Hz, 1H), 6.36 (t, J = 52 Hz, 1H), 4.52-4.43 (m, 2H), 4.13 (br s, 1H), 3.55-3.52 (m, 1H), 3.25-3.22 (m, 1H), 3.05-2.96 (m, 2H), 2.89-2.83 (m, 4H), 2.78 (s, 6H), 2.67 - 2.53 (br s, 2H), 2.13 (s, 1H), 1.89-1.79 (m, 2H), 1.53 (br s, 1H), 1.08-1.00 (m, 2H), 0.76- 0.69 (m, 2H) |

### Example 376

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-cyclopropyl-3-((1-methylpiperidin-4-yl)oxy)thiophene-2-carboxamide

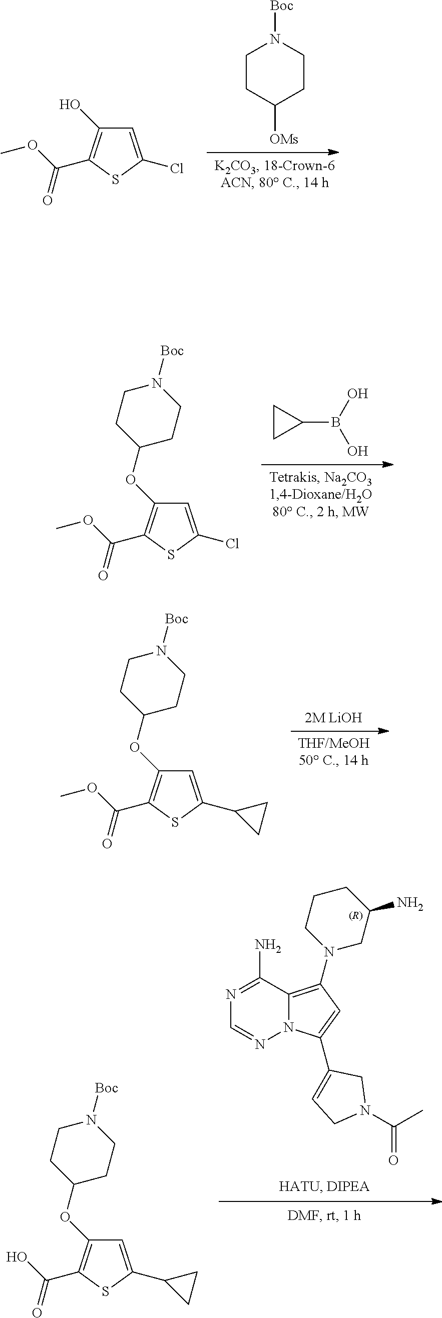

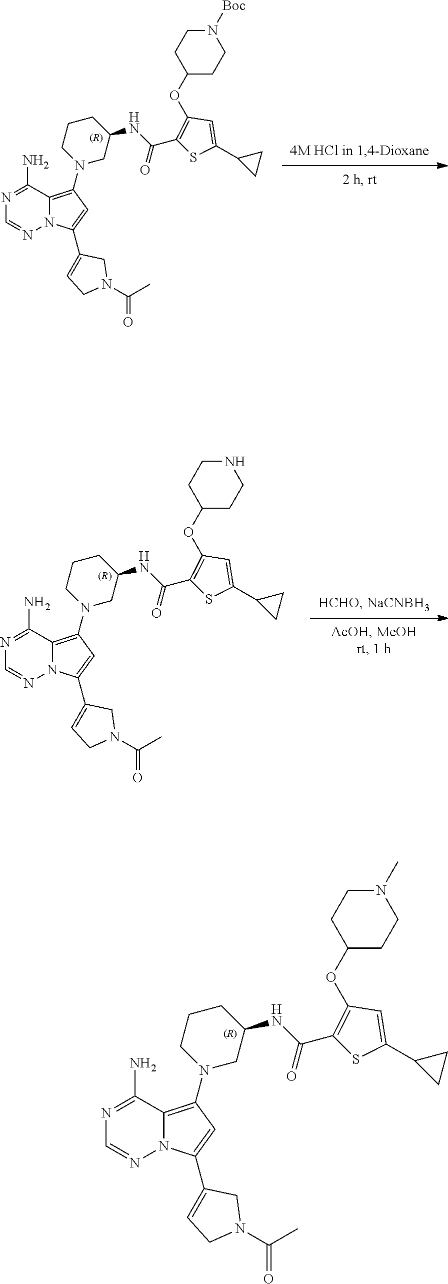

### Example 376A. tert-butyl 4-((5-chloro-2-(methoxycarbonyl)thiophen-3-yl)oxy)piperidine-1-carboxylate

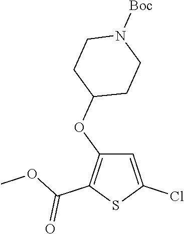 [SMILES: COC(=O)c1sc(Cl)cc1OC1CCN(C(=O)OC(C)(C)C)CC1]

Methyl 5-chloro-3-hydroxythiophene-2-carboxylate (1.0 g, 5.19 mmol), K₂CO₃ (1.44 g, 10.38 mmol), 8-crown-6 (0.069 g, 0.260 mmol) and tert-butyl 4-((methylsulfonyl)oxy) piperidine-1-carboxylate (2.90 g, 10.38 mmol) were taken in acetonitrile (10 mL). Reaction mixture was stirred at 80° C. for 14 h. After cooling, water (50 mL) and ethyl acetate (70 mL) were added. Organic phase was separated, dried over Na₂SO₄, filtered and concentrated. The crude product was purified by silica-gel chromatography (6% MeOH in DCM) which afforded Example 376A (1.2 g, 62% yield) as a yellow liquid. MS: [M−56]⁺=320; ¹H NMR (400 MHz, CDCl₃) δ ppm 6.74 (s, 1H), 4.54-4.47 (m, 1H), 3.94-3.83 (m, 4H), 3.64 (ddd, J=4.3, 8.3, 13.0 Hz, 2H), 3.56-3.46 (m, 2H), 2.07 (s, 1H), 1.97-1.78 (m, 5H), 1.49 (s, 9H).

### Example 376B. tert-butyl 4-((5-cyclopropyl-2-(methoxycarbonyl)thiophen-3-yl)oxy)piperidine-1-carboxylate

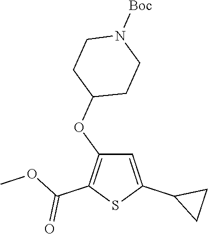 [SMILES: COC(=O)c1sc(C2CC2)cc1OC1CCN(C(=O)OC(C)(C)C)CC1]

Analogous to the procedure described in Example 370A, reaction of Example 376A (0.8 g, 2.13 mmol) and cyclopropylboronic acid (1.28 g, 14.9 mmol) afforded Example 376B (0.58 g, 71% yield) as a yellow gummy solid. MS: [M−Boc+2H]⁺=282; ¹H NMR (400 MHz, CDCl₃) δ ppm 6.55 (s, 1H), 4.53 (quin, J=4.8 Hz, 1H), 3.88-3.81 (m, 3H), 3.71-3.58 (m, 2H), 3.56-3.46 (m, 2H), 2.12-2.01 (m, 2H), 1.95-1.79 (m, 4H), 1.53-1.42 (m, 9H), 1.14-1.05 (m, 2H), 0.93-0.77 (m, 2H).

### Example 376C. 3-((1-(tert-butoxycarbonyl)piperidin-4-yl)oxy)-5-cyclopropylthiophene-2-carboxylic acid

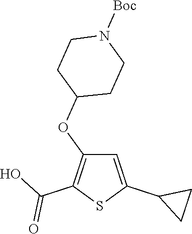 [SMILES: CC(C)(C)OC(=O)N1CCC(Oc2cc(C3CC3)sc2C(=O)O)CC1]

Analogous to the procedure described in Example 370B, reaction of Example 376B (0.58 g, 1.52 mmol) and lithium hydroxide monohydrate (0.109 g, 4.56 mmol) afforded Example 376C (0.45 g, 81% yield) as an off-white solid. MS: [M+H]⁺=368.2; ¹H NMR (400 MHz, DMSO-d₆) δ ppm 6.86 (s, 1H), 4.66-4.57 (m, 1H), 3.59-3.40 (m, 3H), 3.32-3.21 (m, 7H), 2.14-2.07 (m, 1H), 1.80 (dt, J=3.8, 8.6 Hz, 2H), 1.65-1.50 (m, 2H), 1.41 (s, 10H), 1.10-1.01 (m, 2H), 0.81-0.73 (m, 2H).

### Example 376D. tert-butyl (R)-4-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-cyclopropylthiophen-3-yl)oxy)piperidine-1-carboxylate

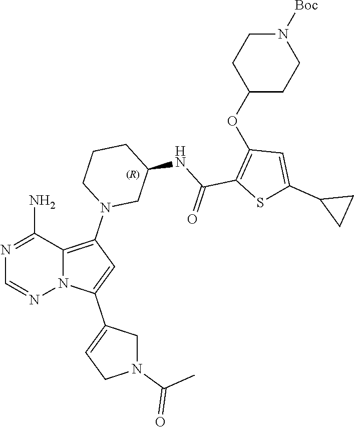

Example 376C (250 mg, 0.719 mmol), 3-((1-(tert-butoxycarbonyl)piperidin-4-yl)oxy)-5-cyclopropylthiophene-2-carboxylic acid lithium salt (264 mg, 0.719 mmol), HATU (44.3 mg, 0.116 mmol) and DIEA (0.092 mL, 0.53 mmol) were taken in DMF (3 mL). The reaction mixture was stirred at rt for 1 h followed by the addition of water (15 mL) and ethyl acetate (30 mL). Organic phase was separated, dried over Na₂SO₄, filtered and concentrated. The crude product was purified by silica-gel chromatography (5% MeOH in DCM) which afforded Example 376D (40 mg, 51% yield) as a yellow solid. MS: [M+H]=691.2.

### Example 376E. (R)—N-(1-(7-(1-acetyl-2,5-dihydro-1H-pyrrol-3-yl)-4-aminopyrrolo[2,1-f][1,2,4]triazin-5-yl)piperidin-3-yl)-5-cyclopropyl-3-(piperidin-4-yloxy)thiophene-2-carboxamide

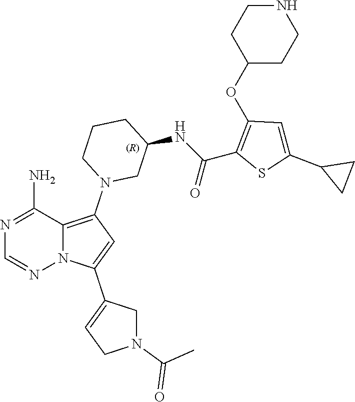

Treatment of Example 376D (40 mg, 0.058 mmol) with 4N HCl in 1,4-dioxane (0.145 mL, 0.579 mmol) afforded Example 376E. MS: [M+H]⁺=591.4, HPLC Method E: RT=7.17 min; HPLC Method F: RT=8.76 min; ¹H NMR (400 MHz, DMSO-d₆) δ ppm 8.03 (br s, 1H), 7.87 (d, J=2.0 Hz, 1H), 7.40 (br s, 1H), 6.99-6.83 (m, 1H), 6.83-6.67 (m, 3H), 4.69 (br s, 1H), 4.61-4.42 (m, 3H), 4.27 (br s, 1H), 4.14 (br s, 1H), 3.26-3.05 (m, 2H), 3.03-2.85 (m, 3H), 2.82 (br s, 1H), 2.76-2.62 (m, 2H), 2.47-2.23 (m, 2H), 2.14-1.96 (m, 4H), 1.92 (br s, 2H), 1.82 (br s, 2H), 1.54-1.44 (m, 2H), 1.09-1.00 (m, 2H), 0.78-0.70 (m, 2H).

### Example 376

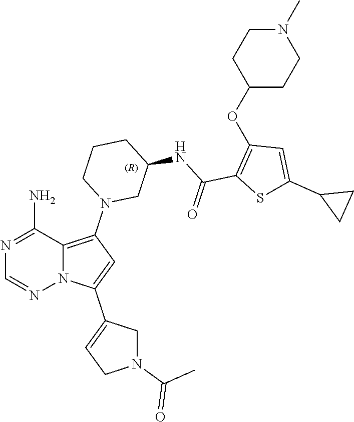

Formaldehyde (aq. 38% solution, 0.029 mL, 0.399 mmol) was added to a solution of Example 376E (50 mg, 0.08 mmol) and acetic acid (1 drop) in methanol (2 mL) at rt. After stirring for 10 min, sodium cyanoborohydride (10.0 mg, 0.159 mmol) was added and mixture was stirred for another 1 h followed by concentration to dryness. The residue was suspended in water (15 mL) and extracted with ethyl acetate (30 mL). Organic layer was separated, dried over Na₂SO₄, filtered and concentrated. Resulted residue was purified by silica-gel column chromatography to afford Example 376 (16 mg, 35% yield). LC-MS Method E: RT=1.39 min, [M+H]⁺=599.2; LC-MS Method F: RT=0.99 min, [M+H]⁺=599.2; ¹H NMR (400 MHz, DMSO-d₅) δ ppm 8.02 (br s, 1H), 7.86 (d, J=1.5 Hz, 1H), 7.33 (br s, 1H), 6.91 (s, 1H), 6.82-6.73 (m, 2H), 6.71 (d, J=12.5 Hz, 1H), 4.73-4.52 (m, 2H), 4.47 (br d, J=4.8 Hz, 2H), 4.31-4.21 (m, 1H), 4.14 (br s, 1H), 3.20-3.14 (m, 1H), 2.95 (br s, 1H), 2.78 (br s, 1H), 2.54 (br s, 1H), 2.33 (td, J=1.7, 3.6 Hz, 2H), 2.19 (s, 3H), 2.12-2.06 (m, 1H), 2.05-1.88 (m, 6H), 1.85-1.61 (m, 3H), 1.48-1.47 (m, 1H), 1.56-1.34 (m, 1H), 1.09-0.97 (m, 2H), 0.77-0.64 (m, 2H).

The following examples in Table 25 were prepared using the same procedure as shown in Example 376. Example 376C was coupled with the appropriate amines, followed by Boc removal and reductive amination.

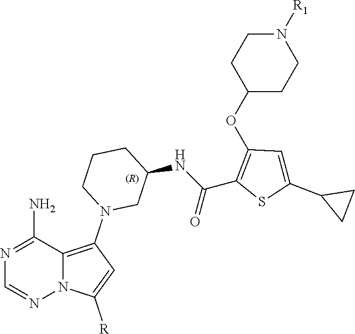

TABLE 25

| | | | | | HPLC | |
|---|---|---|---|---|---|---|
| | | | | | Method, | |
| Ex- | | | | LCMS | RT | |
| ample | R | R1 | Name | (M + H)⁺ | (min.) | ¹H NMR |
| 377 | 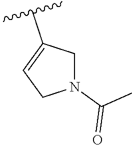 [SMILES: *C1=CCN(C(C)=O)C1] | Isopropyl | (R)-N-(1-(7-(1-acetyl- 2,5-dihydro-1H-pyrrol- 3-yl)-4- aminopyrrolo[2,1- f][1,2,4]triazin-5- yl)piperidin-3-yl)-5- cyclopropyl-3-((1- isopropylpiperidin-4- yl)oxy)thiophene-2- carboxamide | 633.3 | E: 1.42 F: 1.04 | (400 MHz, DMSO-d₆) δ ppm 8.02 (br s, 1H), 7.86 (d, J = 1.5 Hz, 1H), 7.33 (br s, 1H), 6.91 (s, 1H), 6.82-6,73 (m, 2H), 6.71 (d, J = 12.5 Hz, 1H), 4.81- 4.51 (m, 2H), 4.54-4.44 (m, 2H), 4.29-4. 11 (m, 3H), 3.45 (br d, J = 5.3 Hz, 1H), 3.09 (br s, 2H), 2.99 (br d, J = 13.3 Hz, 3H), 2.35 (br d, J = 4.0 Hz, 2H), 2.24-1.97 (m, 6H), 1.94-1.75 (m, 4H), 1.25 (d, J = 6.5 Hz, 6H), 1.18-0.98 (m, 2H), 1.09-0.97 (m, 2H), 0.77-0.64 (m, 2H). |
| 378 | 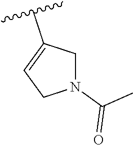 [SMILES: *C1=CCN(C(C)=O)C1] | Cyclo- propyl | (R)-N-(1-(7-(1-acetyl- 2,5-dihydro-1H-pyrrol- 3-yl)-4- aminopyrrolo[2,1- f][1,2,4]triazin-5- yl)piperidin-3-yl)-5- cyclopropyl-3-((1- cyclopropylpiperidin-4- yl)oxy)thiophene-2- carboxamide | 631.3 | E: 1.77 F: 1.03 | (400 MHz, DMSO-d₆) δ ppm 8.02 (br s, 1H), 7.86 (d, J = 1.5 Hz, 1H), 7.33 (br s, 1H), 6.91 (s, 1H), 6.82-6.73 (m, 2H), 6.71 (d, J = 12.5 Hz, 1H), 4.73- 4.42 (m, 3H), 4.26 (br d, J = 2.3 Hz, 1H), 4.13 (br s, 1H), 3.90 (s, 2H), 3.22-3.13 (m, 1H), 2.92 (br s, 1H), 2.84-2.65 (m, 3H), 2.45 (br dd, J = 4.6, 5.6 Hz, 1H), 2.14-1.96 (m, 4H), 1.93-1.74 (m, 5H), 1.70- 1.44 (m, 4H), 1.09-0.97 (m, 2H), 0.77-0.67 (m, 4H), 0.47-0.17 (m, 4H) |
| 379 | 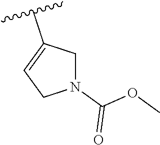 [SMILES: *C1=CCN(C(=O)OC)C1] | H | Methyl (R)-3-(4-amino-5- (3-(5-cyclopropyl-3- (piperidin-4- yloxy)thiophene-2- carboxamido)piperidin-1- yl)pyrrolo[2,1- f][1,2,4]triazin-7-yl)-2,5- dihydro-1H-pyrrole-1- carboxylate | 607.2 | E: 4.34 F: 5.81 | (400 MHz, DMSO-d₆) δ ppm 8.02 (br s, 1H), 7.86 (s, 1H), 7.39 (br s, 1H), 6.91 (s, 1H), 6.82-6.66 (m, 3H), 4.62-4.45 (m, 3H), 4.29 (br s, 2H), 4.14 (br s, 1H), 3.66 (d, J = 4.0 Hz, 3H), 3.17 (br s, 1H), 3.02- 2.84 (m, 4H), 2.81 (br s, 1H), 2.74-2.60 (m, 5H), 2.14-2.07 (m, 1H), 1.92 (br s, 2H), 1.86-1.75 (m, 2H), 1.48 (br s, 3H), 1.08-1.01 (m, 2H), 0.76-0.70 (m, 2H) |
| 380 | 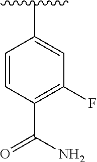 [SMILES: *c1ccc(C(N)=O)c(F)c1] | H | (R)-N-(1-(4-amino-7-(4- carbamoyl-3- fluorophenyl)pyrrolo[2,1- f][1,2,4]triazin-5- yl)piperidin-3-yl)-5- cyclopropyl-3-(piperidin-4- yloxy)thiophene-2- carboxamide | 619.2 | G: 8.17 K: 9.45 | (400 MHz, DMSO-d₆) δ ppm 8.15 (br d, J = 1.4 Hz, 1H), 8.01 (dd, J = 1.5, 8.3 Hz, 1H), 7.92 (s, 1H), 7.74 (t, J = 8.1 Hz, 1H), 7.65 (s, 1H), 7.62 (s, 1H), 7.41 (br s, 1H), 7.27 (s, 1H), 6.97-6.81 (m, 2H), 4.54 (td, J = 4.4, 8.4 Hz, 1H), 4.17 (br s, 1H), 3.00 (br s, 1H), 2.95-2.82 (m, 3H), 2.75 (br s, 1H), 2.71- 2.64 (m, 3H), 2.21-2.00 (m, 1H), 1.93 (br s, 5H), 1.49 (br s, 3H), 1.10-1.00 (m, 2H), 0.76-0.71 (m, 2H) |
| 381 | 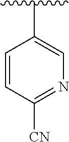 [SMILES: *c1ccc(C#N)nc1] | Me | (R)-N-(1-(4-amino-7-(6- cyanopyridin-3- yl)pyrrolo[2,1- f][1,2,4]triazin-5- yl)piperidin-3-yl)-5- cyclopropyl-3-((1- methylpiperidin-4- yl)oxy)thiophene-2- carboxamide | 598.3 | E: 1.68 F: 1.16 | (400 MHz, DMSO-d₆) δ ppm 9.61 (s, 1H), 9.42 (d, J = 1.8 Hz, 1H), 8.81 (dd, J = 2.3, 8.3 Hz, 1H), 8.24 (br d, J = 7.5 Hz, 2H), 8.10 (d, J = 8.3 Hz, 1H), 7.96 (s, 1H), 7.45-7.34 (m, 1H), 6.96-6.81 (m, 1H), 4.78-4.57 (m, 1H), 4.23-4.07 (m, 1H), 3.54-3.47 (m, 1H), 3.10-2.87 (m, 4H), 2.83-2.69 (m, 4H), 2.38- 2.33 (m, 1H), 2.19-1.67 (m, 6H), 1.54 (br s, 1H), 1.16 (t, J = 7.3 Hz, 3H), 1.10-1.00 (m, 2H), 0.76- 0.71 (m, 2H) |
| 382 | 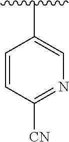 [SMILES: *c1ccc(C#N)nc1] | Isopropyl | (R)-N-(1-(4-amino-7-(6- cyanopyridin-3- yl)pyrrolo[2,1- f][1,2,4]triazin-5- yl)piperidin-3-yl)-5- cyclopropyl-3-((1- isopropylpiperidin-4- yl)oxy)thiophene-2- carboxamide | 626.3 | E: 1.23 F: 1.74 | (400 MHz, DMSO-d₆) δ ppm 9.42 (d, J = 1.8 Hz, 1H), 8.81 (dd, J = 2.1, 8.4 Hz, 1H), 8.23 (s, 1H), 8.09 (d. J = 8.3 Hz, 1H), 7.95 (s, 1H), 7.40 (s, 2H), 6.90 (s, 2H), 4.60 (br s, 1H), 4.27-4.06 (m, 1H), 3.25-3.17 (m, 1H), 3.08-2.99 (m, 2H), 2.74 (br s, 4H), 2.15-2.06 (m, 2H), 2.02-1.71(m, 6H), 1.48 (br s, 1H), 1.32-0.82 (m, 10H), 0,76-0.70 (m, 2H) |

### Example 383

### Preparation of (R)—N-(1-(4-amino-7-(6-cyanopyridin-3-yl)pyrrolo[2,1-f][1,2,4]triazin-5-yl)piperidin-3-yl)-5-chloro-3-((1-(2,2-difluoroethyl)azetidin-3-yl)oxy)thiophene-2-carboxamide

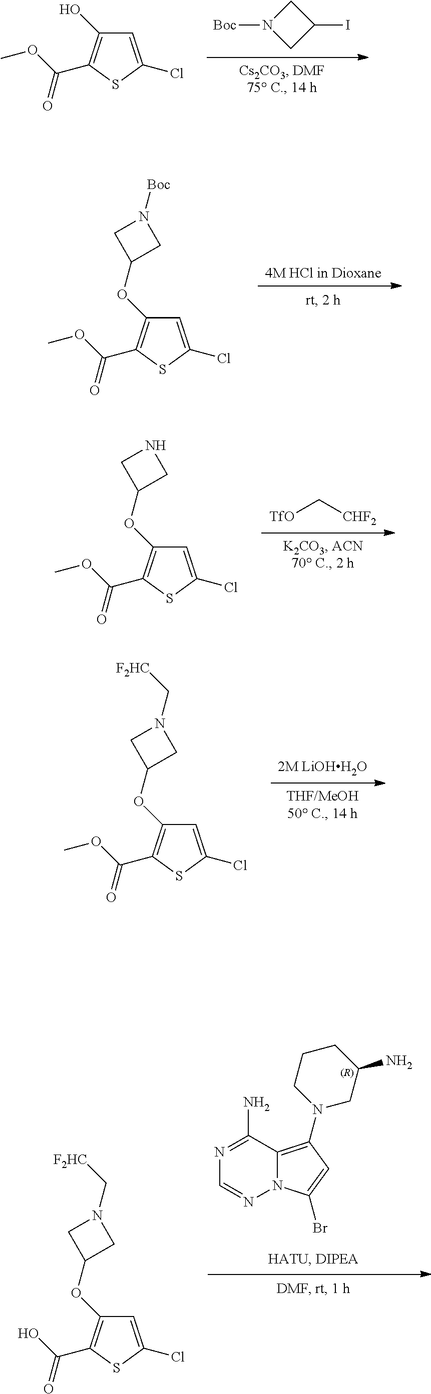

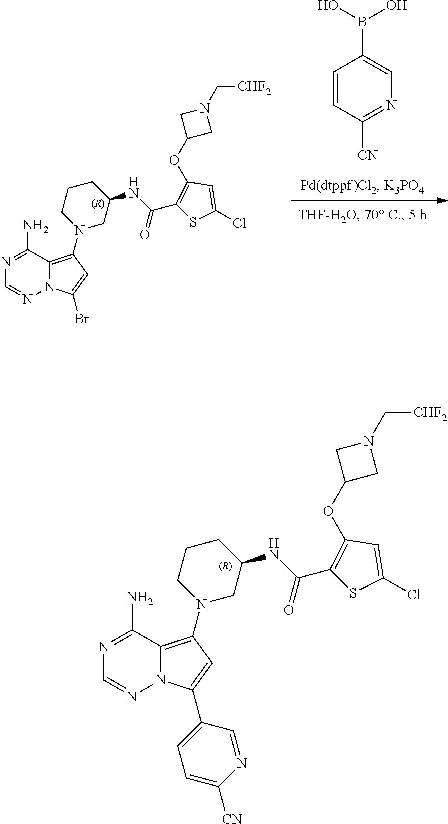

### Example 383A. tert-butyl 3-((5-chloro-2-(methoxycarbonyl)thiophen-3-yl)oxy)azetidine-1-carboxylate

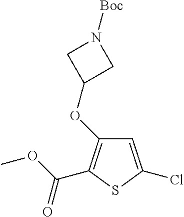 [SMILES: COC(=O)c1sc(Cl)cc1OC1CN(C(=O)OC(C)(C)C)C1]

To a solution of methyl 5-chloro-3-hydroxythiophene-2-carboxylate (1.0 g, 5.19 mmol) and tert-butyl 3-iodoazetidine-1-carboxylate (2.94 g, 10.38 mmol) in DMF (10 mL), was added Cs₂CO₃ (3.38 g, 10.4 mmol). The reaction mixture was stirred at 70° C. for 14 h. After cooling, water (50 mL) and ethyl acetate (50 mL) were added. Organic phase was separated, dried over Na₂SO₄, filtered and concentrated. The crude product was purified by silica-gel chromatography (5% MeOH in DCM) to afford Example 383A (1.5 g, 83% yield) as a colorless gummy liquid. MS: [M−56]⁺=292.2; ¹H NMR (400 MHz, CDCl₃) δ ppm 6.46 (s, 1H), 4.92 (tt, J=4.2, 6.3 Hz, 1H), 4.34-4.25 (m, 2H), 4.16-4.09 (m, 2H), 3.85 (s, 3H), 1.47 (s, 9H).

### Example 383B. Methyl 3-(azetidin-3-yloxy)-5-chlorothiophene-2-carboxylate

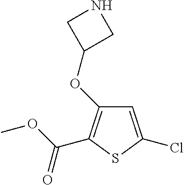

To a stirred solution of Example 383A (0.7 g, 2.013 mmol) in 1,4-dioxane (7 mL), was added 4N HCl in 1,4-dioxane (5.03 mL, 20.13 mmol). Reaction mixture was stirred at it for 2 h and concentrated to afford the Example 383B (0.35 g, 70% yield) as a fine yellow color solid. MS: [M−56]⁺=248.1; ¹H NMR (400 MHz, CDCl₃) δ ppm 6.62 (s, 1H), 4.34 (t, J=4.5 Hz, 1H), 3.84 (s, 3H), 3.73-3.61 (m, 4H).

### Example 383C. Ethyl 5-chloro-3-((1-(2,2-difluoroethyl)azetidin-3-yl)oxy)thiophene-2-carboxylate

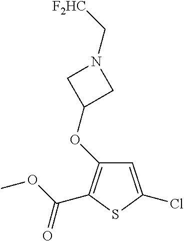 [SMILES: COC(=O)c1sc(Cl)cc1OC1CN(CC(F)F)C1]

To a solution of Example 383B (200 mg, 0.807 mmol and 2,2-difluoroethyl trifluoromethanesulfonate (0.216 mL, 1.62 mmol) in acetonitrile (4 mL), was added K₂CO₃ (223 mg, 1.62 mmol). Reaction mixture was stirred at 70° C. for 2 h followed by the addition of with water (20 mL) and ethyl acetate (30 mL). Organic phase was separated, dried over Na₂SO₄, filtered and concentrated. The crude product was purified by silica gel chromatography (3% MeOH in DCM) to afford Example 383C (0.15 g, 60% yield) as a colorless liquid. MS: [M+H]⁺=312.2; ¹H NMR (400 MHz, CDCl₃) δ ppm 6.50 (s, 1H), 5.93 (t, J=4.3 Hz, 1H), 0.85 (quin, J=5.9 Hz, 1H), 3.97-3.90 (m, 2H), 3.85 (s, 3H), 3.44-3.38 (m, 2H), 2.92 (dt, J=4.3, 15.1 Hz, 2H).

### Example 383D. 5-chloro-3-((1-(2,2-difluoroethyl)azetidin-3-yl)oxy)thiophene-2-carboxylic acid, lithium salt

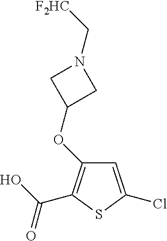 [SMILES: O=C(O)c1sc(Cl)cc1OC1CN(CC(F)F)C1]

Solution of Example 383C (0.55 g, 1.76 mmol) and 2M lithium hydroxide monohydrate (3 mL, 5.29 mmol) were heated at 50° C. for 14 h. Reaction mixture was concentrated to dryness to afford Example 383D (0.48 g, 89% yield) as an off-white solid. MS: [M+H]⁺=298.1; ¹H NMR (400 MHz, DMSO-d₆) δ ppm 6.73 (s, 1H), 6.18-6.00 (m, 1H), 6.18-5.96 (t, J=4.0 Hz, 1H), 4.92 (quin, J=5.6 Hz, 1H), 3.68 (dd, J=6.3, 8.3 Hz, 2H), 3.32-3.17 (m, 2H), 2.86 (dt, J=4.3, 16.1 Hz, 2H).

### Example 383E. (R)—N-(1-(4-amino-7-bromopyrrolo[2,1-f][1,2,4]triazin-5-yl)piperidin-3-yl)-5-chloro-3-((1-(2,2-difluoroethyl)azetidin-3-yl)oxy)thiophene-2-carboxamide

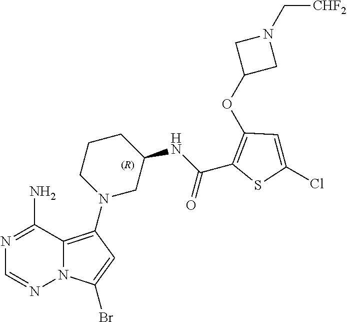

Example 383D, 5-chloro-3-((1-(2,2-difluoroethyl)azetidin-3-yl)oxy)thiophene-2-carboxylic acid lithium salt (0.5 g, 1.5 mmol), (R)-5-(3-aminopiperidin-1-yl)-7-bromopyrrolo[2,1-f][1,2,4]triazin-4-amine (0.52 g, 1.50 mmol), HATU (0.626 g, 1.65 mmol) and DIEA (0.784 mL, 4.49 mmol) were suspended in anhydrous DMF (5 mL). Reaction mixture was stirred at rt for 1 h followed by the addition of with water (25 mL) and ethyl acetate (50 mL). Organic phase was separated, dried over Na₂SO₄, filtered and concentrated. The crude product was purified by silica-gel chromatography (5% MeOH in DCM) to afford the Example 383E (0.5 g, 57% yield) as a yellow solid. MS: [M+H]⁺=592.2.

### Example 383

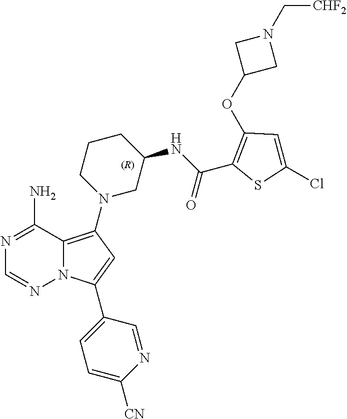

Analogous to the procedure described for Example 276, reaction of Example 383E (45 mg, 0.076 mmol) and 5-(4,4,5,5-tetramethyl-1,3,2-dioxaborolan-2-yl)picolinonitrile (22.8 mg, 0.099 mmol) afforded Example 383 (6.1 mg, 25% yield). LC-MS Method E: RT=1.98 min, [M+H]⁺=614.2; LC-MS Method F: RT=1.27 min, [M+H]⁺=614.1; ¹H NMR (400 MHz, DMSO-d₆) δ ppm 9.47-9.37 (m, 1H), 8.80 (dd, J=2.0, 8.3 Hz, 1H), 8.24 (br s, 1H), 8.10 (d, J=7.8 Hz, 1H), 7.96 (d, J=1.5 Hz, 1H), 7.46-7.27 (m, 2H), 7.15 (s, 1H), 6.20 (s, 1H), 5.13-5.03 (m, 1H), 4.83 (br d, J=3.3 Hz, 1H), 4.23-3.98 (m, 3H), 3.70-3.55 (m, 3H), 3.17 (br s, 1H), 2.93 (br dd, J=6.8, 12.8 Hz, 4H), 1.99-1.75 (m, 3H), 1.57 (s, 1H).

The following examples in Table 26 were prepared using the same procedure as shown in Example 383. Example 383E was coupled with the appropriate boronic acids or boronate esters to afford Examples 384-387.

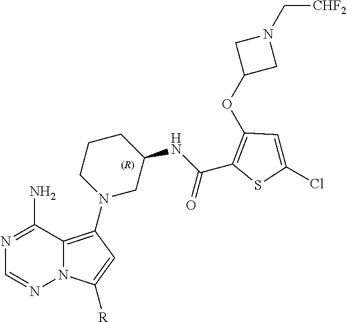

TABLE-26

| | | | LCMS | HPLC | |
|---|---|---|---|---|---|
| | | | (M + H)⁺ | Method, | |
| Example | R | Name | | RT (min.) | ¹H NMR |
| 384 | 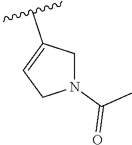 [SMILES: *C1=CCN(C(C)=O)C1] | (R)-N-(1-(7-(1-acetyl-2,5- dihydro-1H-pyrrol-3-yl)-4- aminopyrrolo[2,1- f][1,2,4]triazin-5-yl)piperidin-3- yl)-5-chloro-3-((1-(2,2- difluoroethyl)azetidin-3- yl)oxy)thiophene-2-carboxamide | 621.3 | E: 1.56 F: 0.96 | (400 MHz, DMSO-d₆) δ ppm 11.30 (s, 1H), 9.86 (br s, 1H), 7.91 (s, 1H), 7.74 (br d, J = 7.8 Hz, 2H), 7.42 (d, J = 8.3 Hz, 1H), 7.29-7.23 (m, 1H), 7.23-6.93 (m, 3H), 6.80-6.70 (m, 2H), 4.62-4.50 (m, 4H), 4.33-4.23 (m, 3H), 3.65 (d, J = 3.8 Hz, 3H), 3.54-3.51 (m, 2H), 3.30-3.27 (m, 1H), 3.07-3.02 (m, 1H), 2.94 (br s, 6H), 2.73 (br s, 2H), 1.87 (br s, 3H), 1.57 (br s, 1H) |
| 385 | 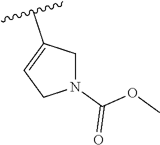 [SMILES: *C1=CCN(C(=O)OC)C1] | Methyl (R)-3-(4-amino-5-(3-(5- chloro-3-((1-(2,2- difluoroethyl)azetidin-3- yl)oxy)thiophene-2- carboxamido)piperidin-1- yl)pyrrolo[2,1-f][1,2,4]triazin-7- yl)-2,5-dihydro-1H-pyrrole-1- carboxylate | 637.3 | E: 1.81 F: 1.11 | (400 MHz, DMSO-d₆) δ ppm 7.92 (s, 1H), 7.35 (br d, J = 3.4 Hz, 1H), 7.25-7.07 (m, 2H), 6.97 (s, 1H), 6.81-6.70 (m, 2H), 5.09 (br d, J = 5.9 Hz, 1H), 4.57- 4.48 (m, 2H), 4.31 (br dd, J = 5.0, 6.7 Hz, 5H), 3.66 (d, J = 3.9 Hz, 3H), 3.18 (s, 1H), 3.04-2.97 (m, 2H), 2.88-2.76 (m, 2H), 1.95-1.78 (m, 3H) |
| 386 | 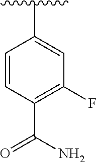 [SMILES: *c1ccc(C(N)=O)c(F)c1] | (R)-N-(1-(4-amino-7-(4- carbamoyl-3- fluorophenyl)pyrrolo[2,1- f][1,2,4]triazin-5-yl)piperidin-3- vl)-5-chloro-3-((1-(2,2- difluoroethyl)azetidin-3- yl)oxy)thiophene-2-carboxamide | 649.2 | E: 1.64 F: 1.00 | (400 MHz, DMSO-d₆) δ ppm 8.12 (dd, J = 1.2, 13.0 Hz, 1H), 8.01 (dd, J = 1.7, 8.3 Hz, 1H), 7.94 (s, 1H), 7.75 (t, J = 8.1 Hz, 1H), 7.70-7.61 (m, 2H), 7.27 (s, 1H), 7.16 (s, 1H), 6.14-5.84 (m, 1H), 5.06 (br d, J = 9.5 Hz, 1H), 4.20 (br s, 1H), 3.91 (s, 2H), 3.18 (s, 2H), 3.04-2.83 (m, 4H), 1.86 (br dd, J = 2.0, 3.2 Hz, 3H), 1.72-1.57 (m, 1H) |
| 387 | 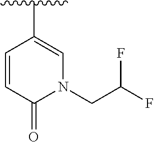 [SMILES: *c1ccc(=O)n(CC(F)F)c1] | (R)-N-(1-(4-amino-7-(1-(2,2- difluoroethyl)-6-oxo-1,6- dihydropyridin-3-yl)pyrrolo[2,1- f][1,2,4]triazin-5-yl)piperidin-3- yl)-5-chloro-3-((1-(2,2- difluoroethyl)azetidin-3- yl)oxy)thiophene-2-carboxamide | 669.2 | E: 1.76 F: 1.51 | (400 MHz, DMSO-d₆) δ ppm 8.49 (d, J = 2.5 Hz, 1H), 8.10 (dd, J = 2.5, 9.5 Hz, 1H), 7.82 (s, 1H), 7.49 (br s, 1H), 7.14 (s, 1H), 6.93 (s, 1H), 6.56 (d, J = 9.8 Hz, 1H), 6.51-6.21 (m, 1H), 6.07-5.76 (m, 1H), 5.06- 4.96 (m, 1H), 4.51-4.36 (m, 2H), 4.24-4.09 (m, 1H), 3.80-3.73 (m, 2H), 3.28-3.11 (m, 3H), 3.00-2.79 (m, 4H), 1.91-1.55 (m, 4H) |

### Example 388

### Preparation of methyl (R)-3-(4-amino-5-(3-(3-(azetidin-3-yloxy)-5-cyclopropylthiophene-2-carboxamido)piperidin-1-yl)pyrrolo[2,1-f][1,2,4]triazin-7-yl)-2,5-dihydro-1H-pyrrole-1-carboxylate

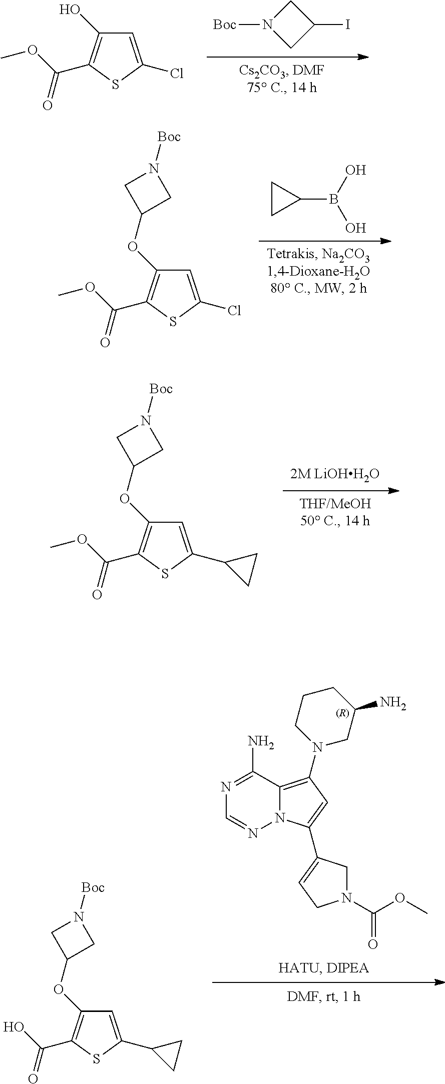

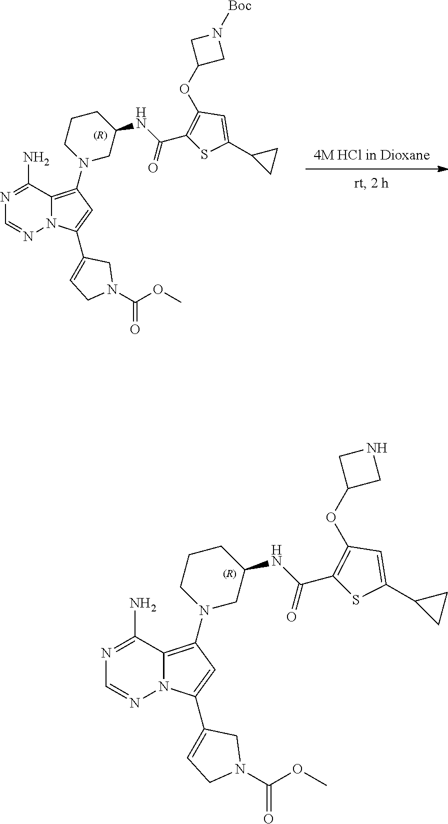

### Example 388A. tert-butyl 3-((5-chloro-2-(methoxycarbonyl)thiophen-3-yl)oxy)azetidine-1-carboxylate

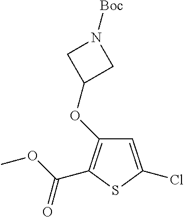 [SMILES: COC(=O)c1sc(Cl)cc1OC1CN(C(=O)OC(C)(C)C)C1]

Analogous to the procedure for the preparation of Example 385A, reaction of methyl 5-chloro-3-hydroxythiophene-2-carboxylate (1.0 g, 5.19 mmol) afforded Example 388A (1.5 g, 83% yield) as a colorless gummy liquid. MS: [M−56]=292.2.

### Example 388B. tert-butyl 3-((5-cyclopropyl-2-(methoxycarbonyl)thiophen-3-yl)oxy)azetidine-1-carboxylate

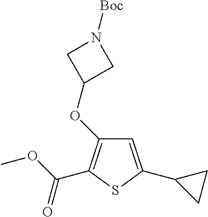 [SMILES: COC(=O)c1sc(C2CC2)cc1OC1CN(C(=O)OC(C)(C)C)C1]

Analogous to the procedure for the preparation of Example 370A, reaction of Example 388A (0.8 g, 2.31 mmol) and cyclopropylboronic acid afforded Example 388B (0.8 g, 87% yield) as a yellow gummy solid. MS: [M+H]⁺=298.0.

### Example 388C. 3-((1-(tert-butoxycarbonyl)azetidin-3-yl)oxy)-5-cyclopropylthiophene-2-carboxylic acid, Lithium salt

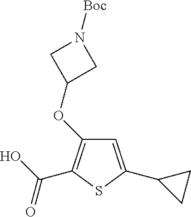 [SMILES: CC(C)(C)OC(=O)N1CC(Oc2cc(C3CC3)sc2C(=O)O)C1]

Analogous to the procedure for the preparation of Example 370B, reaction of Example 388B (0.8 g, 2.264 mmol) and Lithium hydroxide monohydrate (0.163 g, 6.79 mmol) afforded Example 388C (0.7 g, 91% yield) as an off-white solid. MS: [M−56]⁺=284.

### Example 388D. Methyl (R)-3-(4-amino-5-(3-(3-((1-(tert-butoxycarbonyl)azetidin-3-yl)oxy)-5-cyclopropylthiophene-2-carboxamido)piperidin-1-yl)pyrrolo[2,1-f][1,2,4]triazin-7-yl)-2,5-dihydro-1H-pyrrole-1-carboxylate

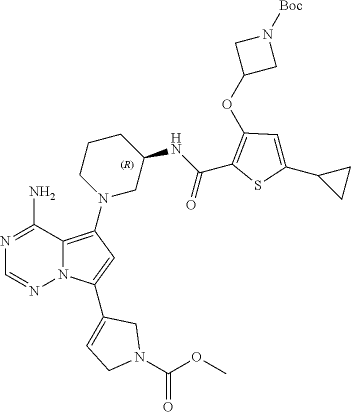

Analogous to the procedure for the preparation of Example 376D, reaction of Example 388C (200 mg, 0.508 mmol) and Intermediate 31 (172 mg, 0.508 mmol) afforded Example 388D (0.12 g, 55% yield) as a yellow solid. MS: [M+H]⁺=679.6.

### Example 388

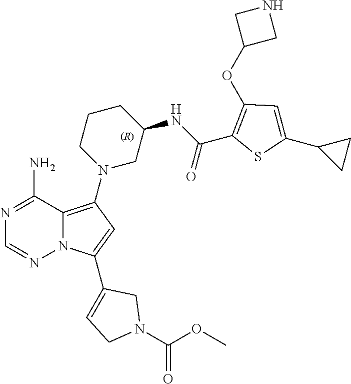

Example 388D (50 mg, 0.074 mmol) was treated with 4N HCl in 1,4-dioxane (0.092 mL, 0.368 mmol) to afford Example 388 (8.9 mg, 20% yield). MS: [M+H]⁺=579.3; HPLC Method E: RT=1.33 min; HPLC Method F: RT=1.05 min; ¹H NMR (400 MHz, DMSO-d₆) δ ppm 8.20 (br s, 1H), 7.86 (s, 1H), 7.41 (s, 1H), 6.80-6.71 (m, 2H), 6.63-6.61 (m, 2H), 5.14 (br t, J=5.9 Hz, 1H), 4.59-4.45 (m, 2H), 4.34-4.08 (m, 2H), 3.93-3.86 (m, 1H), 3.70-3.52 (m, 6H), 3.17-3.05 (m, 2H), 2.96-2.71 (m, 3H), 2.10 (td, J=4.4, 8.5 Hz, 1H), 1.81 (br s, 4H), 1.11-0.98 (m, 2H), 0.80-0.62 (m, 2H).

### Example 389A

### Preparation of tert-butyl (R)-3-((2-((1-(4-amino-7-(4-carbamoyl-3-fluorophenyl)pyrrolo[2,1-f][1,2,4]triazin-5-yl)piperidin-3-yl)carbamoyl)-5-cyclopropylthiophen-3-yl)oxy)azetidine-1-carboxylate

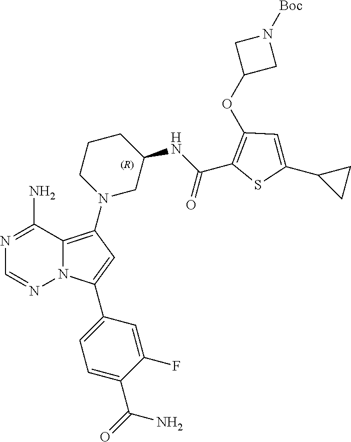

Analogous to the procedure for the preparation of Example 388D, reaction of Example 388B and corresponding amine afforded Example 389A (50 mg, 58% yield) as a yellow solid. MS: [M+H]⁺=691.5.

### Example 389

### Preparation of (R)—N-(1-(4-amino-7-(4-carbamoyl-3-fluorophenyl)pyrrolo[2,1-f][1,2,4]triazin-5-yl)piperidin-3-yl)-3-(azetidin-3-yloxy)-5-cyclopropylthiophene-2-carboxamide

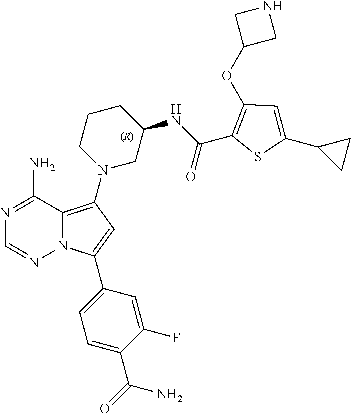

Analogous to the procedure for the preparation of Example 388, reaction of Example 389 and 4N HCl in dioxane afforded Example 390 (8 mg, 18% yield) as a yellow solid. MS: [M+H]⁺=591.2; HPLC Method E: RT=1.18 min; ¹H NMR (400 MHz, DMSO-d₆) δ ppm 8.13 (br d, J=13.2 Hz, 2H), 8.03-7.98 (m, 2H), 7.93 (s, 1H), 7.75 (t, J=7.7 Hz, 1H), 7.66 (td, J=1.5, 6.6 Hz, 2H), 7.47 (br s, 1H), 7.27 (s, 1H), 6.86 (br s, 1H), 6.66-6.57 (m, 1H), 5.18-5.11 (m, 1H), 4.31-4.15 (m, 2H), 4.11-4.02 (m, 1H), 3.72 (br dd, J=6.1, 7.6 Hz, 2H), 2.85 (s, 3H), 2.18-2.06 (m, 1H), 1.90-1.77 (m, 2H), 1.40 (s, 3H), 1.06-1.02 (m, 2H), 0.79-0.62 (m, 2H).

### Example 391

### N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpyrrolidin-2-yl)methoxy)thiophene-2-carboxamide

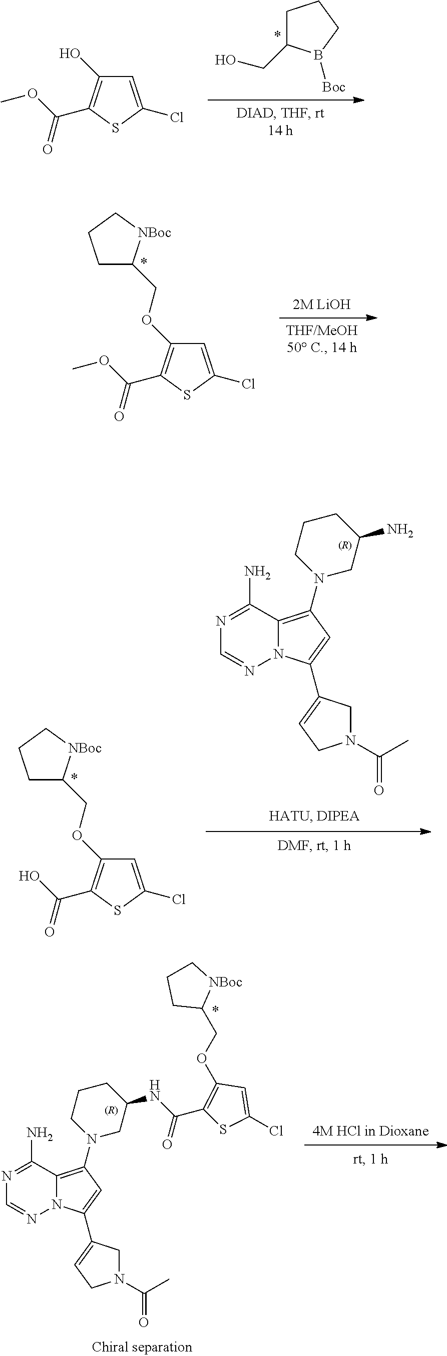

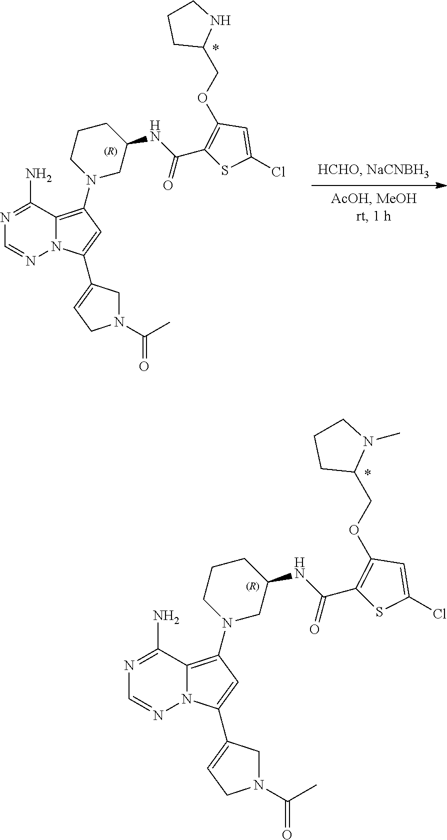

### Example 391A. tert-butyl 2-(((2-chloro-5-(methoxycarbonyl)thiophen-3-yl)oxy)methyl)pyrrolidine-1-carboxylate

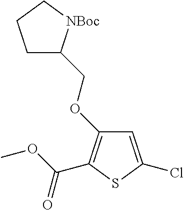 [SMILES: COC(=O)c1sc(Cl)cc1OCC1CCCN1C(=O)OC(C)(C)C]

Analogous to the procedure described for Example 276A, reaction of methyl 5-chloro-4-hydroxythiophene-2-carboxylate (1.0 g, 5.19 mmol) and tert-butyl 2-(hydroxymethyl)pyrrolidine-1-carboxylate (1.045 g, 5.19 mmol) afforded Example 391A (1.2 g, 61.5% yield) as a colorless gummy liquid. MS: [M−56]⁺=276; ¹H NMR (400 MHz, CDCl₃) δ ppm 6.92 (s, 1H), 6.81 (br s, 1H), 6.99-6.77 (m, 1H), 4.30-4.19 (m, 2H), 4.13-3.96 (m, 2H), 3.47-3.32 (m, 2H), 2.14 (br s, 2H), 2.06 (br d, J=7.5 Hz, 2H), 1.87 (br s, 1H), 1.54-1.45 (m, 1H).

### Example 391B. 3-((1-(tert-butoxycarbonyl)pyrrolidin-2-yl)methoxy)-5-chlorothiophene-2-carboxylic acid, Lithium salt

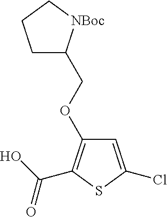 [SMILES: CC(C)(C)OC(=O)N1CCCC1COc1cc(Cl)sc1C(=O)O]

Analogous to the procedure described for Example 276B, reaction of Example 391A (1.2 g, 3.19 mmol) and lithium hydroxide monohydrate (0.4 mL, 9.58 mmol) afforded Example 116B (1.0 g, 91% yield) as an off-white solid. MS: [M−Boc+2H]⁺=262.2.

### Example 391C. tert-butyl 2-(((2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)methyl)pyrrolidine-1-carboxylate

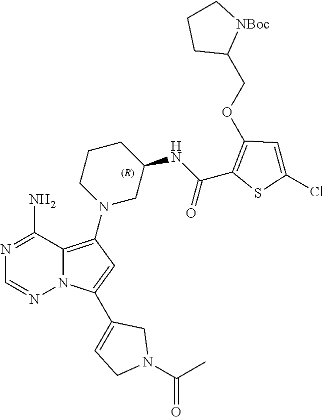

Analogous to the procedure for the preparation of Example 376D, reaction of Example 391B (250 mg, 0.662 mmol) and Intermediate 32 afforded Example 391C as diastereomeric mixture. Two diastereomers were separated by chiral chromatography.

Diastereomer-1: 150 mg, 30% Yield. MS: [M+H]⁺=685.3. Chiral SFC purity-100% at rt=4.67 min [Chiralcel ODH (250×4.6) mm, 5u; % CO₂: 60%; % Co solvent: 40% (5 mM NH₄OH in MeOH+MeCN (1:1)); Total Flow: 4 g/min; Back Pressure: 100 bars; Temperature: 35° C.; Detection: UV at 250 nm].

Diastereomer-2: 160 mg 40% Yield. MS: [M+H]⁺=685.3. Chiral SFC purity-99.4% at rt=6.47 min [Chiralcel ODH (250×4.6) mm, 5u; % CO₂: 60%; % Co solvent: 40% (5 mM NH₄OH in MeOH+MeCN (1:1)); Total Flow: 4 g/min; Back Pressure: 100 bars; Temperature: 35° C.; Detection: UV at 250 nm].

### Example 391D. N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-(pyrrolidin-2-ylmethoxy)thiophene-2-carboxamide, Diastereomer 1

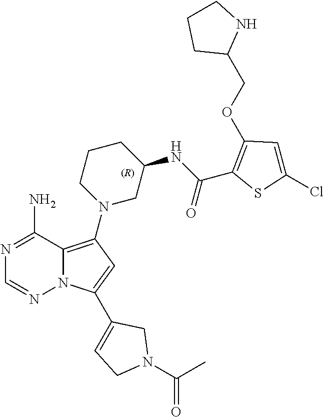

Reaction of Example 391C (Iso-1, 30 mg, 0.044 mmol) and 4N HCl in dioxane (0.055 mL, 0.219 mmol) afforded Example 391D (11 mg, 43% Yield). MS: [M+H]⁺=585.2; HPLC Method E: RT=1.29 min; HPLC Method F: RT=0.95 min; ¹H NMR (400 MHz, DMSO-d₆) δ ppm 8.02 (br s, 1H), 7.87 (d, J=0.8 Hz, 1H), 7.69 (br s, 1H), 7.34 (d, J=1.3 Hz, 1H), 6.77 (br d, J=9.5 Hz, 1H), 6.70 (d, J=11.8 Hz, 2H), 4.69 (br d, J=1.5 Hz, 1H), 4.53-4.42 (m, 2H), 4.27 (br d, J=1.8 Hz, 1H), 4.22-4.10 (m, 2H), 4.05 (br d, J=8.8 Hz, 1H), 3.49-3.45 (m, 1H), 3.19-3.07 (m, 3H), 3.01-2.92 (m, 2H), 2.88-2.71 (m, 3H), 2.03 (d, J=15.8 Hz, 3H), 1.94-1.50 (m, 7H), 1.41 (br s, 1H).

### Example 391E. N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-(pyrrolidin-2-ylmethoxy)thiophene-2-carboxamide, Diastereomer 2

Reaction of Example 391C (Iso-2) and 4N HCl in dioxane afforded Example 391E (8 mg, 35% Yield). MS: [M+H]⁺=585.2; HPLC Method E: RT=1.29 min; HPLC Method F: RT=0.98 min; ¹H NMR (400 MHz, DMSO-d₆) δ ppm 8.03 (s, 1H), 7.87 (s, 1H), 7.61 (br s, 1H), 7.35 (s, 1H), 6.81-6.74 (m, 1H), 6.70 (d, J=11.5 Hz, 2H), 4.69 (br s, 1H), 4.53-4.44 (m, 2H), 4.30-4.20 (m, 2H), 4.18-4.04 (m, 2H), 3.64-3.54 (m, 1H), 3.19-3.13 (m, 1H), 2.97-2.97 (m, 1H), 3.04-2.87 (m, 3H), 2.76 (br s, 1H), 2.03 (d, J=16.0 Hz, 3H), 1.97-1.65 (m, 7H), 1.50 (br s, 2H).

### Example 391. N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpyrrolidin-2-yl)methoxy)thiophene-2-carboxamide, Diastereomer 1

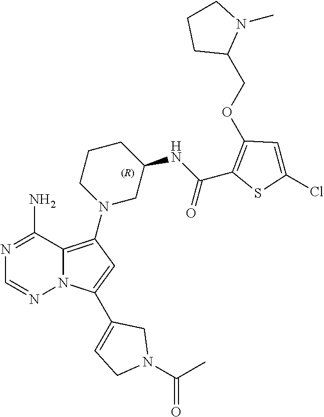

Analogous to the procedure for the preparation of Example 376, reaction formaldehyde (aq. 38% solution 0.029 mL, 0.399 mmol) and Example 391D (40 mg, 0.064 mmol) afforded the Example 391 (20 mg, 50% Yield). MS: [M+H]⁺=599.2; LC-MS Method E: RT=1.63 min, [M+H]⁺=599.2; LC-MS Method F: RT=0.97 min. [M+H]⁺=599.3; ¹H NMR (400 MHz, DMSO-d₆) δ ppm 8.02 (br s, 1H), 7.87 (d, J=1.3 Hz, 1H), 7.53 (br s, 1H), 7.37 (d, J=1.8 Hz, 1H), 6.82-6.75 (m, 1H), 6.70 (d, J=15.0 Hz, 2H), 4.72-4.64 (m, 1H), 4.47 (br dd, J=1.4, 7.4 Hz, 2H), 4.32-4.23 (m, 2H), 4.11 (br d, J=10.3 Hz, 2H), 3.18 (d, J=5.3 Hz, 1H), 2.96 (br s, 2H), 2.78 (br s, 2H), 2.55 (br s, 2H), 2.27 (br dd, J=2.3, 4.0 Hz, 2H), 2.03 (d, J=16.0 Hz, 3H), 1.82 (br s, 4H), 1.65 (br s, 4H).

### Example 392

### Preparation of (R)—N-(1-(4-amino-7-(6-cyanopyridin-3-yl)pyrrolo[2,1-f][1,2,4]triazin-5-yl)piperidin-3-yl)-4-(2-(dimethylamino)ethoxy)-1-(4-methoxyphenyl) pyrazole-5-carboxamide

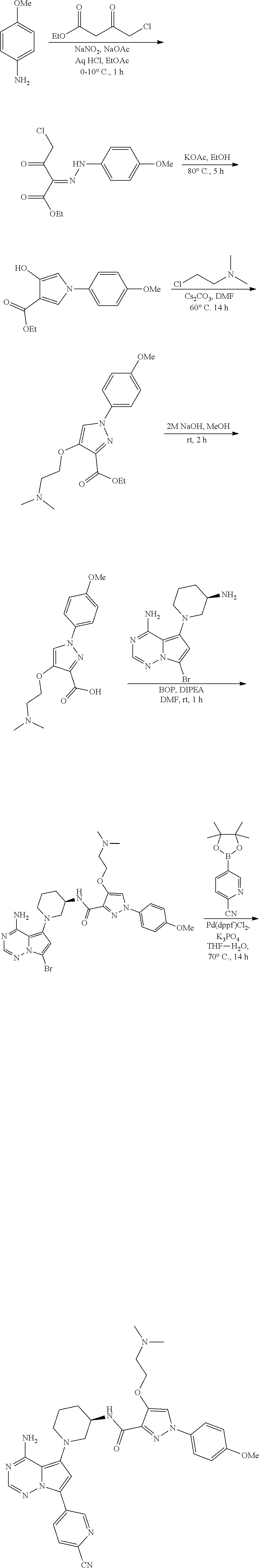

### Example 392A. Ethyl (Z)-4-chloro-2-(2-(4-methoxyphenyl)hydrazineylidene)-3-oxobutanoate

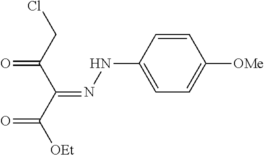 [SMILES: CCOC(=O)/C(=N/Nc1ccc(OC)cc1)C(=O)CCl]

4-methoxyaniline (5.0 g, 40.6 mmol) was taken in hydrochloric acid (37%, 45 mL) and cooled at 0° C. Then, sodium nitrite (2.94 g, 42.6 mmol) in water (20 mL) was added and after 10 min, solution of ethyl 4-chloro-3-oxobutanoate (16.7 mL, 122 mmol) and sodium acetate (50.0 g, 609 mmol) in water (20 mL)-ethanol (30 mL) was added dropwise at 0° C. The mixture was stirred at 0° C. for 1 h and the yellow powder precipitated was filtered, washed with water and dried to afford Example 392A (10 g, 82% yield) as a yellow solid. MS: [M+H]⁺=299.1.

### Example 392B. Ethyl 4-hydroxy-1-(4-methoxyphenyl)-1H-pyrazole-3-carboxylate

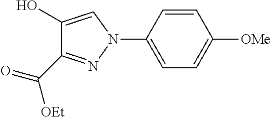 [SMILES: CCOC(=O)c1nn(-c2ccc(OC)cc2)cc1O]

Potassium acetate (6.57 g, 67.0 mmol) was added to a solution of Example 392A (10 g, 33.5 mmol) in ethanol (100 mL). Reaction mixture was stirred at 80° C. for 14 h. After cooling, it was concentrated under reduced pressure and upon addition of water (100 mL), the solids precipitated was filtered and dried to afford Example 392B (7 g, 80% yield) as light brown solid. MS: [M+H]⁺=263.1; ¹H NMR (400 MHz, DMSO-d₆) δ ppm 9.06 (s, 1H), 7.97 (s, 1H), 7.75-7.70 (m, 2H), 7.08-7.03 (m, 2H), 4.30 (q, J=7.0 Hz, 2H), 3.32 (s, 3H), 1.31 (t, J=7.0 Hz, 3H).

### Example 392C. Ethyl 4-(2-(dimethylamino)ethoxy)-1-(4-methoxyphenyl)-1H-pyrazole-3-carboxylate

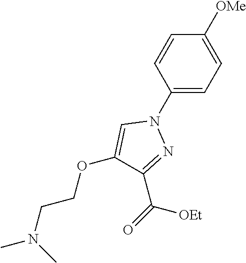 [SMILES: CCOC(=O)c1nn(-c2ccc(OC)cc2)cc1OCCN(C)C]

To a solution of Example 392B (0.5 g, 1.906 mmol) and 2-chloro-N, N-dimethylethan-1-amine (0.410 g, 3.81 mmol) in DMF (5 mL), was added Cs₂CO₃ (1.242 g, 3.81 mmol). Reaction mixture was stirred at 75° C. for 14 h. After cooling, water (25 mL) and ethyl acetate (50 mL) were added. Organic layer was separated, concentrated, dried over Na₂SO₄, filtered and concentrated to afford Example 392C (0.3 g, 50% yield) as a yellow solid. MS: [M+H]⁺=334.3.

### Example 392D. 4-(2-(dimethylamino)ethoxy)-1-(4-methoxyphenyl)-1H-pyrazole-3-carboxylic acid

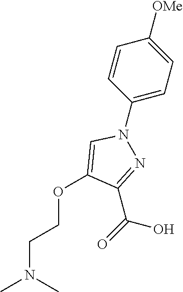 [SMILES: COc1ccc(-n2cc(OCCN(C)C)c(C(=O)O)n2)cc1]

Sodium hydroxide (2M solution, 0.900 ml, 1.800 mmol) was added to a solution of Example 392C (200 mg, 0.600 mmol) in ethanol (4 mL). Reaction mixture was stirred at rt for 2 h. It was concentrated to afford Example 392D (180 mg, 88% yield) as a brown gummy solid. MS: [M+H]⁺=306.1.

### Example 392E. (R)—N-(1-(4-amino-7-bromopyrrolo[2,1-f][1,2,4]triazin-5-yl)piperidin-3-yl)-4-(2-(dimethylamino)ethoxy)-1-(4-methoxyphenyl)-1H-pyrazole-5-carboxamide

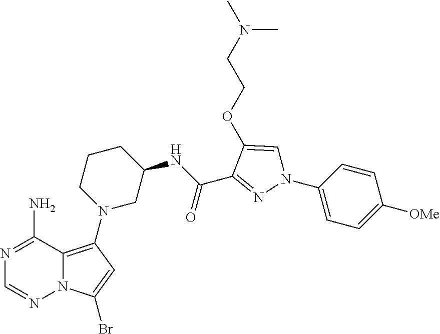 [SMILES: COc1ccc(-n2cc(OCCN(C)C)c(C(=O)N[C@@H]3CCCN(c4cc(Br)n5ncnc(N)c45)C3)n2)cc1]

Analogous to the procedure for the preparation of Example 370C, reaction of Example 392D (0.145 g, 0.475 mmol) and (R)-5-(3-aminopiperidin-1-yl)-7-bromopyrrolo[2,1-f][1,2,4]triazin-4-amine afforded Example 392E (0.14 g, 54% yield) as a yellow solid. MS: [M+2]⁺=600.3; ¹H NMR (400 MHz, DMSO-d₆) δ ppm 9.91-9.17 (m, 1H), 8.47 (s, 1H), 8.18-7.92 (m, 2H), 7.86-7.78 (m, 3H), 7.10 (d, J=9.0 Hz, 2H), 6.78 (s, 1H), 4.35 (br d, J=4.5 Hz, 2H), 4.17 (br s, 1H), 3.82 (s, 3H), 3.53-3.35 (m, 2H), 3.15 (br d, J=9.0 Hz, 1H), 2.99 (br d, J=11.5 Hz, 1H), 2.91 (s, 6H), 2.56-2.53 (m, 3H), 1.93-1.72 (m, 1H), 1.59 (br s, 1H).

### Example 392

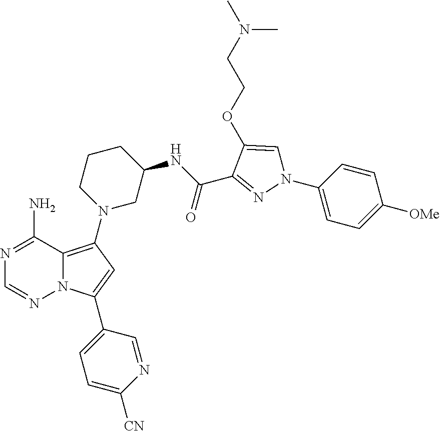 [SMILES: COc1ccc(-n2cc(OCCN(C)C)c(C(=O)N[C@@H]3CCCN(c4cc(-c5ccc(C#N)nc5)n5ncnc(N)c45)C3)n2)cc1]

Analogous to the procedure for the preparation of Example 376, reaction of Example 392E ((30 mg, 0.050 mmol) and 5-(4,4,5,5-tetramethyl-1,3,2-dioxaborolan-2-yl)picolinonitrile (17.3 mg, 0.075 mmol) afforded Example 392 (6.3 mg, 54% yield) as a yellow solid. MS: [M+H]⁺=622.3; LC-MS Method E: RT=1.62 min, [M+H]⁺=622.3; LC-MS Method F: RT=1.29 min, [M+H]⁺=622.3; ¹H NMR (400 MHz, DMSO-d₆) δ ppm 9.44 (d, J=2.2 Hz, 1H), 8.82 (dd, J=2.2, 8.3 Hz, 1H), 8.41 (s, 1H), 8.15 (d, J=0.7 Hz, 1H), 8.09 (d, J=8.3 Hz, 1H), 7.96 (s, 1H), 7.89-7.81 (m, 1H), 7.80-7.73 (m, 2H), 7.42 (s, 1H), 7.12-7.04 (m, 2H), 7.02-6.99 (br s, 1H), 4.30-4.09 (m, 3H), 3.81 (s, 3H), 3.25-3.16 (m, 2H), 3.08-2.98 (m, 1H), 2.91-2.70 (m, 4H), 2.39-2.26 (m, 6H), 1.98-1.78 (m, 2H), 1.67-1.48 (m, 1H).

### Example 393

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(piperidin-4-yl)thiophene-2-carboxamide

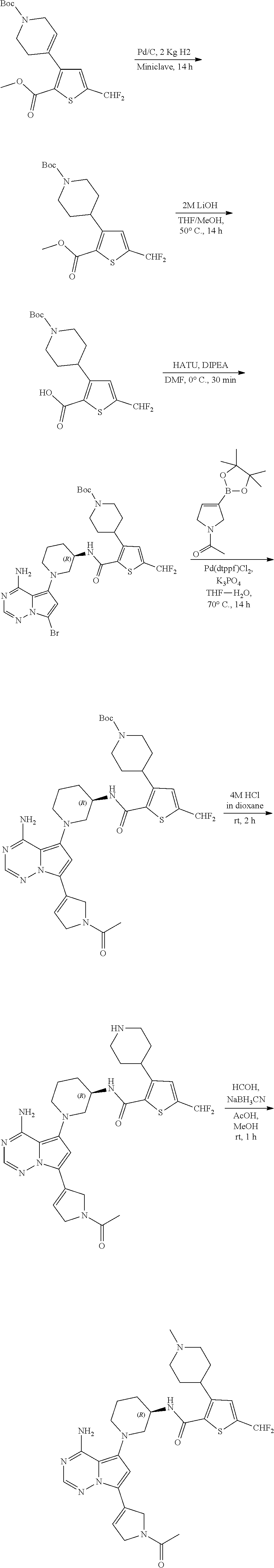

### Example 393A. tert-butyl 4-(5-(difluoromethyl)-2-(methoxycarbonyl)thiophen-3-yl)piperidine-1-carboxylate

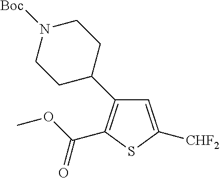 [SMILES: COC(=O)c1sc(C(F)F)cc1C1CCN(C(=O)OC(C)(C)C)CC1]

Example 322A, tert-butyl 4-(5-(difluoromethyl)-2-(methoxycarbonyl)thiophen-3-yl)-3,6-dihydropyridine-1(2H)-carboxylate (1.0 g, 2.68 mmol) in methanol (10 mL) was degassed with N₂. Then, Pd—C(60%) (0.285 g, 1.34 mmol) was added and stirred under 2 kg H₂ pressure for 14 h. Reaction mixture was diluted with excess methanol (50 mL) and filtered through Celite. Filtrate was concentrated to afford the Example 393A (0.8 g, 80% yield) as a gummy solid. MS: [M+H]⁺=276.1.

### Example 393B. 3-(1-(tert-butoxycarbonyl)piperidin-4-yl)-5-(difluoromethyl)thiophene-2-carboxylic acid, lithium salt

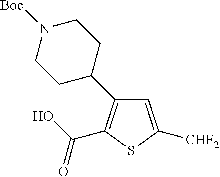 [SMILES: CC(C)(C)OC(=O)N1CCC(c2cc(C(F)F)sc2C(=O)O)CC1]

Analogous to the procedure for the preparation of Example 322B, reaction of Example 393A (0.8 g, 2.131 mmol) and 2M aq. solution of lithium hydroxide monohydrate (0.5 mL, 6.39 mmol) afforded Example 393B (0.7 g, 91% yield) as an off-white solid. MS: [M−H]⁺=360.1.

### Example 393C. tert-butyl (R)-4-(2-((1-(4-amino-7-bromopyrrolo[2,1-f][1,2,4]triazin-5-yl)piperidin-3-yl)carbamoyl)-5-(difluoromethyl)thiophen-3-yl)piperidine-1-carboxylate

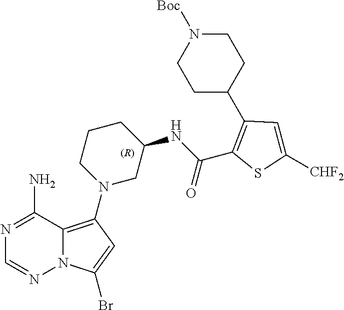

Analogous to the procedure for the preparation of Example 322C, reaction of Example 393B (0.65 g, 1.87 mmol) and (R)-5-(3-aminopiperidin-1-yl)-7-bromopyrrolo[2,1-f][1,2,4]triazin-4-amine (0.689 g, 1.87 mmol) afforded Example 393C (0.75 g, 61% yield) as a yellow solid. MS: [M+H]⁺=654.2; ¹H NMR (400 MHz, CDCl₃) δ ppm 7.93 (s, 1H), 7.22 (s, 1H), 6.86-6.75 (m, 1H), 6.99-6.63 (m, 1H), 6.73-6.62 (m, 1H), 6.62-6.51 (m, 1H), 5.95 (br s, 1H), 4.29 (br s, 1H), 4.23 (br s, 1H), 3.79-3.55 (m, 1H), 3.37 (br s, 1H), 3.12-3.02 (m, 1H), 2.90 (s, 1H), 2.82 (s, 5H), 2.16-2.01 (m, 3H), 2.01-1.77 (m, 6H), 1.55-1.43 (m, 13H), 1.36-1.23 (m, 2H).

### Example 393D. tert-butyl (R)-4-(2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-(difluoromethyl)thiophen-3-yl)piperidine-1-carboxylate

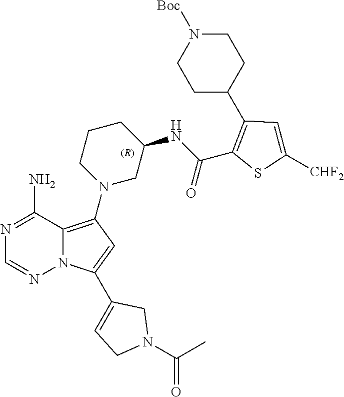

Analogous to the procedure for the preparation of Example 322D, reaction of Example 393C (300 mg, 0.458 mmol) and (3-(4,4,5,5-tetramethyl-1,3,2-dioxaborolan-2-yl)-2,5-dihydro-1H-pyrrol-1-yl) ethan-1-one (130 mg, 0.55 mmol) afforded Example 393D (220 mg, 70% yield) as a yellow solid. [M+2]⁺685.4.

### Example 393E. (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(piperidin-4-yl)thiophene-2-carboxamide

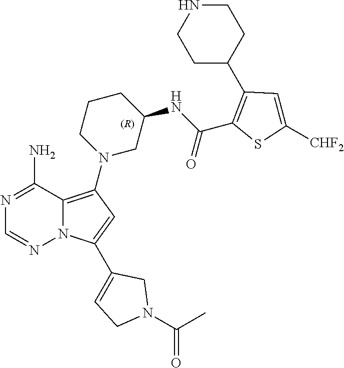

Analogous to the procedure for the preparation of Example 350, reaction of Example 393D (30 mg, 0.044 mmol) and 4N HCl in dioxane (0.055 mL, 0.219 mmol) afforded Example 393E (21.5 mg, 84% yield). LCMS Method E: RT=1.21 min; [M+H]⁺=585.3; HPLC Method F: RT=1.06 min, [M+H]⁺=585.3; ¹H NMR (400 MHz, DMSO-d₆) δ ppm 8.50 (br d, J=1.3 Hz, 2H), 8.20 (br s, 1H), 7.91 (s, 1H), 7.49-7.15 (m, 1H), 7.33 (t, J=55.2 Hz, 1H), 6.82-6.70 (m, 2H), 4.74-4.63 (m, 1H), 4.55-4.40 (m, 2H), 4.33-4.24 (m, 1H), 4.13 (br s, 1H), 3.90 (s, 1H), 3.29 (br d, J=1.3 Hz, 2H), 3.17 (s, 1H), 2.99-2.85 (m, 3H), 2.78-2.67 (m, 2H), 2.03 (d, J=16.8 Hz, 3H), 1.96-1.82 (m, 4H), 1.81-1.68 (m, 3H), 1.53 (br s, 1H).

### Example 393

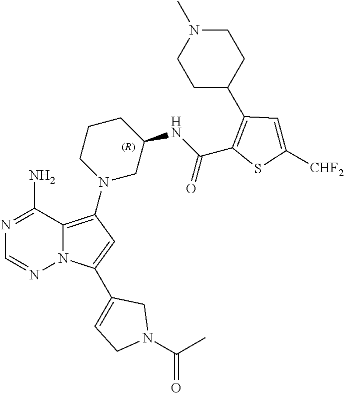

To a solution of Example 393E (50 mg, 0.080 mmol) and acetic acid (0.5 μl, 8 μmol) in methanol (2 mL), was added formaldehyde (38% solution, 0.029 mL, 0.402 mmol). Reaction mixture was stirred at rt for 10 min. Sodium cyanoborohydride (10.1 mg, 0.161 mmol) was added and the mixture was stirred for another 1 h, concentrated to dryness and resultant residue was suspended in water (15 mL) and extracted using ethyl acetate (30 mL). The organic layer dried over Na₂SO₄, filtered, concentrated. The crude product was purified by preparative HPLC to afford Example 393 (19.8 mg, 42% yield). LC-MS Method E: RT=1.22 min, [M+H]⁺=599.2; LC-MS Method F: RT=1.05 min, [M+H]⁺=599.2.

The following examples in Table 27 were prepared using the same procedure as shown in Example 393. Example 393C was coupled with the appropriate boronic acids or boronate esters followed by de-Boc and reductive amination with respective aldehydes/ketones.

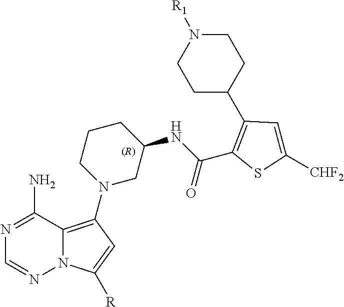

TABLE 27

| | | | | | HPLC | |
|---|---|---|---|---|---|---|
| | | | | LCMS | Method, | |
| Example | R | R1 | Name | (M + H)⁺ | RT (min.) | ¹H NMR |
| 394 | 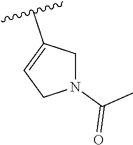 [SMILES: *C1=CCN(C(C)=O)C1] | Isopropyl | (R)-N-(1-(7-(1-acetyl-2,5- dihydro-1H-pyrrol-3-yl)-4- aminopyrrolo[2,1- f][1,2,4]triazin-5- yl)piperidin-3-yl)-5- (difluoromethyl)-3-(1- isopropylpiperidin-4- yl)thiophene-2- | 627.3 | E: 1.29 F: 1.05 | (400 MHz, DMSO-d₆) δ ppm 8.51 (s, 1H), 7.87 (d, J = 2.5 Hz, 1H), 7.43 (s, 1H), 7.34 (t, J = 55.5 Hz, 1H), 6.92 (br s, 1H), 6.77 (br d, J 13.3 Hz, 1H), 6.70 (d, J = 12.3 Hz, 2H), 4.70 (br s, 1H), 4.54-4.44 (m, 2H), 4.27 (br d, J = 2.0 Hz, 2H), 3.19-3.12 (m, 1H), 3.06-2.69 (m, 6H), 2.04 (d, J = 17.3 Hz, 4H), 1.91 (s, 6H), 1.61-1.41 (m, 4H), 0.85 (br s, 6H) |
| 395 | 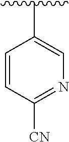 [SMILES: *c1ccc(C#N)nc1] | H | (R)-N-(1-(4-amino-7-(6- cyanopyridin-3- yl)pyrrolo[2,1- f][1,2,4]triazin-5- yl)piperidin-3-yl)-5- (difluoromethyl)-3- (piperidin-4-yl)thiophene- 2-carboxamide | 578.1 | E: 1.45 F: 1.27 | (400 MHz, DMSO-d6) o ppm 9.44 (d, J = 2.0 Hz, 1H), 8.82 (dd, J = 2.1, 8.4 Hz, 1H), 8.55 (br s, 1H), 8.26 (br s, 2H), 8.10 (d, J = 8.3 Hz, 1H), 7.98 (s, 1H), 7.49-7.28 (m, 1H), 7.34 (t, J = 55.5 Hz, 1H), 7.23-6.88 (m, 1H), 4.17 (br s, 1H), 3.34-3.21 (m, 5H), 3.03-2.72 (m, 5H), 2.01-1.84 (m, 4H), 1.82-1.65 (m, 3H), 1.56 (br s, 1H) |
| 396 | 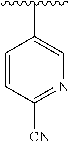 [SMILES: *c1ccc(C#N)nc1] | Me | (R)-N-(1-(4-amino-7-(6- cyanopyridin-3- yl)pyrrolo[2,1- f][1,2,4]triazin-5- yl)piperidin-3-yl)-5- (difluoromethyl)-3-(1- methylpiperidin-4- yl)thiophene-2- carboxamide | 592.2 | E: 1.48 F: 1.27 | (400 MHz, DMSO-d₆) δ ppm 9.45 (d, J = 1.8 Hz, 1H), 8.82 (dd, J = 2.1, 8.4 Hz, 1H), 8.56 (br s, 1H), 8.10 (d, J = 8.3 Hz, 2H), 7.96 (s, 1H), 7.49-7.42 (m, 2H), 7.34 (t, J = 55.5 Hz, 1H), 7.06 (br s, 1H), 4.22 (br d, J = 4.5 Hz, 1H), 3.23-3.17 (m, 2H), 3.07-2.99 (m, 1H), 2.94-2.69 (m, 5H), 2.06 (br d, J = 14.3 Hz, 4H), 1.88-1.83 (m, 1H), 1.80-1.48 (m, 8H) |
| 397 | 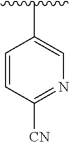 [SMILES: *c1ccc(C#N)nc1] | Isopropyl | (R)-N-(1-(4-amino-7-(6- cyanopyridin-3- yl)pyrrolo[2,1- f][1,2,4]triazin-5- yl)piperidin-3-yl)-5- (difluoromethyl)-3-(1- isopropylpiperidin-4- yl)thiophene-2- carboxamide | 620.3 | E: 1.56 F: 1.27 | |

### Example 398

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(1-methylpyrrolidin-3-yl)thiophene-2-carboxamide

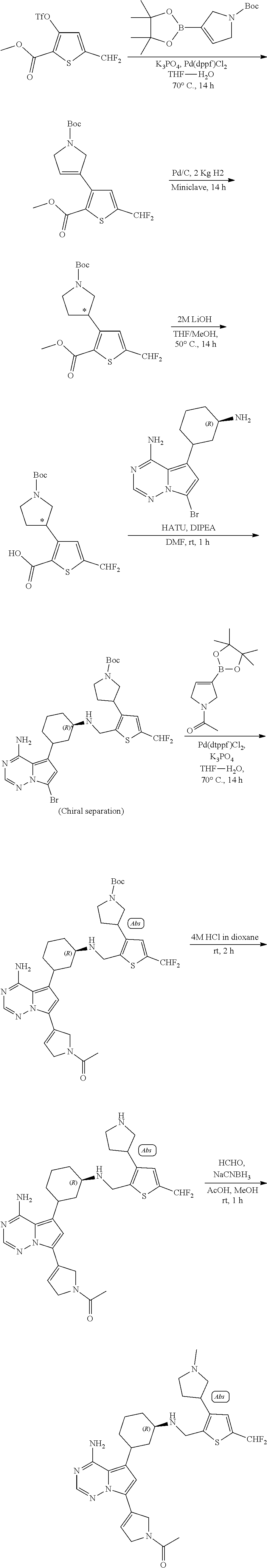

### Example 398A. tert-butyl 3-(5-(difluoromethyl)-2-(methoxycarbonyl)thiophen-3-yl)-2,5-dihydro-1H-pyrrole-1-carboxylate

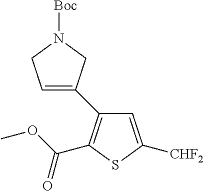 [SMILES: COC(=O)c1sc(C(F)F)cc1C1=CCN(C(=O)OC(C)(C)C)C1]

PdCl₂(dppf)-CH₂Cl₂ adduct (0.168 g, 0.206 mmol) was added to a stirred solution of methyl 5-(difluoromethyl)-3-(((trifluoromethyl)sulfonyl)oxy)thiophene-2-carboxylate (1.0 g, 2.94 mmol), tert-butyl 3-(4,4,5,5-tetramethyl-1,3,2-dioxaborolan-2-yl)-2,5-dihydro-1H-pyrrole-1-carboxylate (1.13 g, 3.82 mmol) and K₃PO₄ (1.2-48 g, 5.88 mmol) in THF (10 mL) and water (1 mL). The mixture was degassed and heated at 70° C. for 14 h. After cooling, water (50 mL) and ethyl acetate (50 mL) were added and the organic phase was separated, dried over Na₂SO₄, filtered and concentrated. The crude product was purified by flash chromatography (5% MeOH in DCM) to afford Example 398A (0.6 g, 56% yield) as a pale yellow solid. MS: [M−Boc+2H]⁺=260.2.

### Example 398B. tert-butyl 3-(5-(difluoromethyl)-2-(methoxycarbonyl)thiophen-3-yl)pyrrolidine-1-carboxylate

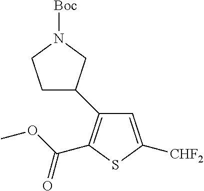 [SMILES: COC(=O)c1sc(C(F)F)cc1C1CCN(C(=O)OC(C)(C)C)C1]

Analogous to the procedure for the preparation of Example 393A, reaction of Example 398A (0.9 g, 2.50 mmol) and Pd—C(60%) (0.267 g, 1.25 mmol) under 2 kg H₂ pressure for 14 h afforded Example 398B (0.8 g, 88% yield) as a pale yellow gummy liquid. MS: [M+H]⁺=362.1.

### Example 398C. 3-(1-(tert-butoxycarbonyl)pyrrolidin-3-yl)-5-(difluoromethyl)thiophene-2-carboxylic acid

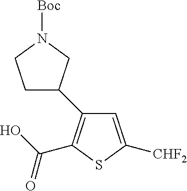 [SMILES: CC(C)(C)OC(=O)N1CCC(c2cc(C(F)F)sc2C(=O)O)C1]

Analogous to the procedure for the preparation of Example 393B, reaction of Example 398B (0.8 g, 2.21 mmol) and lithium hydroxide monohydrate (0.279 g, 6.64 mmol) afforded Example 398C (0.7 g, 91% yield) as an off-white solid. MS: [M−H]⁺=346.3.

### Example 398D. tert-butyl 3-(2-(((R)-1-(4-amino-7-bromopyrrolo[2,1-f][1,2,4]triazin-5-yl)piperidin-3-yl)carbamoyl)-5-(difluoromethyl)thiophen-3-yl)pyrrolidine-1-carboxylate

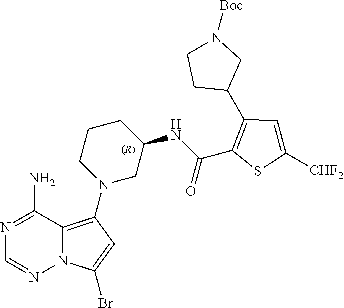

Analogous to the procedure for the preparation of Example 322C, reaction of Example 398C (0.718 g, 1.87 mmol) and (R)-5-(3-aminopiperidin-1-yl)-7-bromopyrrolo[2,1-f][1,2,4]triazin-4-amine (0.65 g, 1.87 mmol) afforded Example 398D (0.75 g, 61% yield) as diastereomeric mixture which was separated by chiral SFC purification.

Diastereomer-1: MS: [M−H]⁺=642.2; Chiral SFC purity-100% at rt=3.81 min [ChiralPak IC (250×4.6 mm, 5 um); % CO₂: 60%; % Co solvent: 40% (5 mM NH₄OH in MeOH+MeCN(1:1)); Total Flow: 4 g/min; Back Pressure: 100 bars; Temperature: 30° C.; Detection: UV at 250 nm].

Diastereomer-2: MS: [M−H]⁺=642.2; Chiral SFC purity-99.8% at rt=6.111 min [ChiralPak IC (250×4.6 mm, 5 um); % CO₂: 60%; % Co solvent: 40% (5 mM NH₄OH in MeOH+MeCN(1:1)); Total Flow: 4 g/min; Back Pressure: 100 bars; Temperature: 30° C.; Detection: UV at 250 nm].

### Example 398E (Diastereomer-1). tert-butyl 3-(2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-(difluoromethyl)thiophen-3-yl)pyrrolidine-1-carboxylate

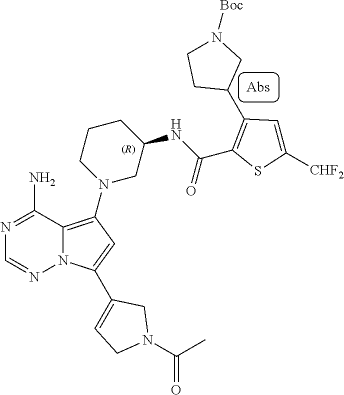

Analogous to the procedure for the preparation of Example 322D, reaction of Example 398D (Diastereomer-1) and (3-(4,4,5,5-tetramethyl-1,3,2-dioxaborolan-2-yl)-2,5-dihydro-1H-pyrrol-1-yl) ethan-1-one afforded (Example 398E, Iso-1) tert-butyl 3-(2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-(difluoromethyl)thiophen-3-yl)pyrrolidine-1-carboxylate (150 mg, 72% yield) as a yellow solid. MS: [M+H]⁺=671.5. Same reaction was performed with Diastereomer-2.

### Example 398F (Diastereomer-1). N—((R)-1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(pyrrolidin-3-yl)thiophene-2-carboxamide

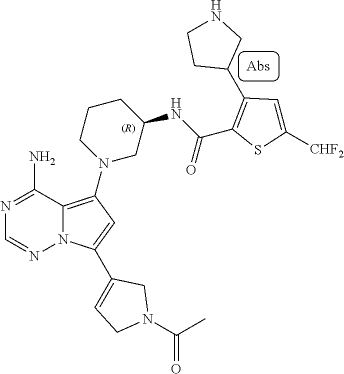

Reaction of Example 398E with 4N HCl in dioxane afforded Example 398F, N—((R)-1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(pyrrolidin-3-yl)thiophene-2-carboxamide (3.7 mg, 15% yield). LCMS Method E: RT=1.15 min, [M+H]⁺=571.3; LCMS Method F: RT=0.96 min, [M+H]⁺=571.3; ¹H NMR (400 MHz, DMSO-d₆) δ ppm 8.57-8.44 (m, 1H), 7.87 (s, 2H), 7.50 (s, 1H), 7.45-7.11 (m, 1H), 6.94 (br s, 1H), 6.78 (br d, J=10.5 Hz, 1H), 6.72 (d, J=11.8 Hz, 1H), 4.69 (br s, 1H), 4.49 (br d, J=10.3 Hz, 2H), 4.27 (br d, J=1.0 Hz, 1H), 4.12 (br s, 1H), 3.72 (br d, J=7.8 Hz, 1H), 3.24-3.13 (m, 2H), 3.04 (br s, 1H), 2.96-2.84 (m, 2H), 2.79-2.63 (m, 3H), 2.14 (br dd, J=4.8, 7.5 Hz, 1H), 2.03 (d, J=17.0 Hz, 3H), 1.88 (s, 3H), 1.81-1.64 (m, 1H), 1.53 (br s, 1H). Same reaction was performed with Diastereomer-2

### Example 398

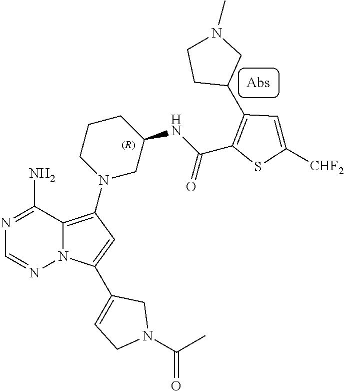

Analogous to the procedure for the preparation of Example 393, reaction of Example 398F and formaldehyde (38% solution) afforded Example 398. LCMS Method E: RT=1.19 min, [M+H]⁺=585.3; LCMS Method F: RT=0.96 min, [M+H]⁺=585.3; ¹H NMR (400 MHz, DMSO-d₆) δ ppm 8.55 (s, 1H), 7.88 (d, J=1.8 Hz, 1H), 7.48 (s, 1H), 7.34 (t, J=55.5 Hz, 1H), 7.26 (br s, 1H), 6.78 (br d, J=11.5 Hz, 1H), 6.72 (d, J=13.8 Hz, 1N), 4.70 (br s, 1H), 4.53-4.44 (m, 2H), 4.30-4.25 (m, 1H), 4.11-4.11 (m, 1H), 4.15-4.05 (m, 1H), 3.17 (br dd, J=1.5, 4.5 Hz, 2H), 2.94 (br s, 2H), 2.73 (br d, J=3.8 Hz, 4H), 2.29 (br s, 4H), 2.04 (d, J=16.8 Hz, 3H), 1.92-1.65 (m, 5H), 1.52 (br s, 1H). Same reaction was performed with Diastereomer-2

The following examples in Table 28 were prepared using the same procedure as shown in Example 398. Both the diastereomers of Example 398D were coupled separately with the appropriate boronic acids or boronate esters, followed by Boc-removal and reductive amination with respective aldehyde/ketone.

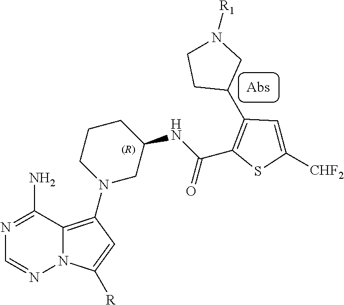

TABLE-28

| | | | | | HPLC | |
|---|---|---|---|---|---|---|
| | | | | LCMS | Method | |
| Example | R | R₁ | Name | (M + H)⁺ | RT (min.) | ¹H NMR |
| 399 | 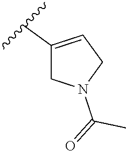 [SMILES: *C1=CCN(C(C)=O)C1] | Isopropyl | N-((R)-1-(7-(1-acetyl- 2,5-dihydro-1H-pyrrol-3- yl)-4-aminopyrrolo[2,1- f][1,2,4]triazin-5- yl)piperidin-3-yl)-5- (difluoromethyl)-3-(1- isopropylpyrrolidin-3- yl)thiophene-2- carboxamide | 613.3 | E: 1.26 F: 1.02 | (400 MHz, DMSO-d₆) δ ppm 8.53 (br d, J = 5.8 Hz, 1H), 8.42 (br s, 1H), 7.88 (s, 1H), 7.50 (br s, 1H), 7.30 (t, J = 53.3 Hz, 1H), 6.93 (br s, 1H), 6.78 (brd, J = 11.0 Hz, 1H), 6.72 (d, J = 12.3 Hz, 1H), 4.70 (br d, J = 2.3 Hz, 1H), 4.49 (br d, J = 10.8 Hz, 2H), 4.27 (br s, 1H), 4.18-4.09 (m, 2H), 3.80 (br s, 2H), 3.20-3.14 (m, 2H), 2.90 (br s, 2H), 2.79-2.69 (m, 2H), 2.42 (s, 1H), 2.17 (br s, 1H), 2.04 (d, J = 16.5 Hz, 3H), 1.88 (br s, 2H), 1.72 (br s, 2H), 1.58-1.45 (m, 1H), 1.02 (br s, 6H) |
| 400 | 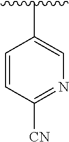 [SMILES: *c1ccc(C#N)nc1] | H | N-((R)-1-(4-amino-7-(6- cyanopyridin-3- yl)pyrrolo[2,1- f][1,2,4]triazin-5- yl)piperidin-3-yl)-5- (difluoromethyl)-3- (pyrrolidin-3- yl)thiophene-2- carboxamide | 564.2 | E: 1.39 F: 1.18 | (400 MHz, DMSO-d₆) δ ppm 9.45 (s, 1H), 8.87- 8.78 (m, 1H), 8.57 (br s, 1H), 8.10 (br d, J = 8.3 Hz, 3H), 7.97 (s, 1H), 7.62-6.99 (m, 4H), 4.21- 4.11 (m, 1H), 3.83-3.78 (m, 1H), 3.58-3.54 (m, 1H), 3.22-3.16 (m, 1H), 3.03-2.92 (m, 2H), 2.86-2.67 (m, 1H), 2.20 (br s, 2H), 1.90 (s, 6H), 1.56 (br s, 1H) |
| 401 | 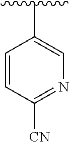 [SMILES: *c1ccc(C#N)nc1] | Me | N-((R)-1-(4-amino-7-(6- cyanopyridin-3- yl)pyrrolo [2,1- f][1,2,4]triazin-5- yl)piperidin-3-yl)-5- (difluoromethyl)-3-(1- methylpyrrolidin-3- yl)thiophene-2- carboxamide | 578.2 | E: 1.47 F: 1.18 | (400 MHz, DMSO-d₆) δ ppm 9.45 (d, J = 2.0 Hz, 1H), 8.86-8.78 (m, 1H), 8.59 (br d, J = 6.5 Hz, 1H), 8.18 (s, 1H), 8.10 (d, J = 8.5 Hz, 1H), 7.97 (s, 1H), 7.50 (d, J = 1.3 Hz, 1H), 7.30 (t, J 53.3 Hz, 1H), 7.20-7.16 (m, 1H), 7.05 (s, 1H), 4.17 (s, 1H), 3.86 (br s, 1H), 3.25-3.21 (m, 3H), 3.00 (br s, 1H), 2.75 (br s, 3H), 2.32-2.17 (m, 4H), 1.91 (s, 3H), 1.71 (s, 2H), 1.53 (br s, 1H) |
| 402 | 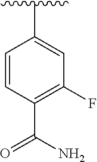 [SMILES: *c1ccc(C(N)=O)c(F)c1] | Me | 5-(4-amino-5-((3R)-3-(5- (difluoromethyl)-3-(1- methylpyrrolidin-3- yl)thiophene-2- carboxamido)piperidin- 1-yl)pyrrolo[2,1- f][1,2,4]triazin-7- yl)picolinamide | 596.3 | E: 1.19 F: 1.009 | (400 MHz, DMSO-d₆) δ ppm 9.31 (d,) = 2.0 Hz, 1H), 8.69 (dd, J = 2.3, 8.3 Hz, 1H), 8.58 (br dd, J = 1.0, 7.3 Hz, 1H), 8.11-8.05 (m, 2H), 7.92 (s, 1H), 7.63 (br s, 1H), 7.48 (s, 1H), 7.44- 7.13 (m, 2H), 7.02 (s, 1H), 4.21-4.08 (m, 1H), 3.83 (br s, 1H), 3.17 (d, J = 4.8 Hz, 1H), 3.00- 2.95 (m, 1H), 2.74 (br s, 3H), 2.29 (br s, 4H), 1.91 (s, 2H), 1.70 (br s, 2H), 1.52 (s, 1H) |
| 403 | 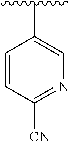 [SMILES: *c1ccc(C#N)nc1] | Isopropyl | N-((R)-1-(4-amino-7-(6- cyanopyridin-3- yl)pyrrolo[2,1- f][1,2,4]triazin-5- yl)piperidin-3-yl)-5- (difluoromethyl)-3-(1- isopropylpyrrolidin-3- yl)thiophene-2- carboxamide | 606.2 | E: 1.52 F: 1.25 | (400 MHz, DMSO-d₆) δ ppm 9.45 (br d, J = 1.5 Hz, 1H), 8.82 (dd, J = 2.0, 8.3 Hz, 1H), 8.51 (br s, 1H), 8.20 (br s, 1H), 8.10 (d, J = 8.3 Hz, 1H), 7.97 (s, 1H), 7.51 (br d, J = 2.3 Hz, 1H), 7.30 (t, J = 53.3 Hz, 1H), 7.20-7.16 (m, 1H), 7.07 (br s, 1H), 4.16 (br s, 1H), 3.84 (br s, 1H), 3.24-3.21 (m, 1H), 3.00 (br s, 1H), 2.97 (br s, 1H), 2.80 (br s, 2H), 2.23-2.16 (m, 2H), 1.91 (br s, 5H), 1.61-1.54 (m, 2H), 1.04 (br s, 6H) |
| 404 | 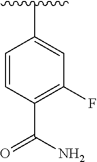 [SMILES: *c1ccc(C(N)=O)c(F)c1] | Isopropyl | 5-(4-amino-5-((3R)-3-(5- (difluoromethyl)-3-(1- isopropylpyrrolidin-3- yl)thiophene-2- carboxamido)piperidin- 1-yl)pyrrolo[2,1- f][1,2,4]triazin-7- yl)picolinamide | 624.3 | E: 1.25 F: 1.03 | (400 MHz, DMSO-d₆) δ ppm 9.31 (d, J = 1.5 Hz, 1H), 8.72-8.67 (m, 1H), 8.49 (s, 1H), 8.09 (d, J = 8.3 Hz, 3H), 7.93 (s, 1H), 7.63 (br d, J = 1.3 Hz, 1H), 7.54-7.50 (m, 1H), 7.42 (t, J = 53.3 Hz, 1H), 7.30 (s, 2H), 4.17 (br d, J = 5.0 Hz, 1H), 3.81 (br s, 1H), 3.12 (br s, 1H), 3.00-2.76 (m, 7H), 2.21-2.15 (m, 1H), 1.91 (s, 3H), 1.81- 1.50 (m, 3H), 1.01 (br d, J = 2.3 Hz, 6H) |
| 405 | 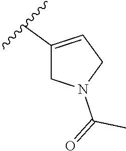 [SMILES: *C1=CCN(C(C)=O)C1] | H | N-((R)-1-(7-(1-acetyl- 2,5-dihydro-1H-pyrrol-3- yl)-4-aminopyrrolo[2,1- f][1,2,4]triazin-5- yl)piperidin-3-yl)-5- (difluoromethyl)-3- (pyrrolidin-3- yl)thiophene-2- carboxamide | 571.2 | E: 1.15 F: 0.96 | (400 MHz, DMSO-d₆) δ ppm 8.89-8.65 (m, 2H), 8.53 (br d, J = 5.8 Hz, 1H), 8.23 (br d, J = 1.0 Hz, 1H), 8.31-8.09 (m, 1H), 7.93 (s, 1H), 7.59 (d, J = 1.3 Hz, 1H), 7.42 (t, J = 53.3 Hz, 1H), 6.84-6.72 (m, 1H), 4.70 (dd, J = 2.1, 3.6 Hz, 1H), 4.55-4.42 (m, 2H), 4.28 (br d, J = 1.0 Hz, 1H), 4.18-4.08 (m, 1H), 4.01-3.92 (m, 1H), 3.58-3.53 (m, 2H), 3.23-3.17 (m, 3H), 3.12-2.97 (m, 3H), 2.73 (br dd, J = 2.5, 4.0 Hz, 1H), 2.33- 2.28 (m, 1H), 2.04 (d, J = 16.8 Hz, 3H), 1.96- 1.73 (m, 4H), 1.56 (s, 1H) |
| 406 | 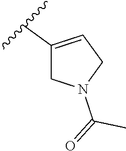 [SMILES: *C1=CCN(C(C)=O)C1] | Me | N-((R)-1-(7-(1-acetyl- 2,5-dihydro-1H-pyrrol-3- yl)-4-aminopyrrolo[2,1- f][1,2,4]triazin-5- yl)piperidin-3-yl)-5- (difluoromethyl)-3-(1- methylpyrrolidin-3- yl)thiophene-2- carboxamide | 585.3 | E: 1.19 F: 0.92 | 1H NMR (400 MHz, DMSO-d₆) δ ppm 8.55 (s, 1H), 7.88 (d, J = 1.8 Hz, 1H), 7.48 (s, 1H), 7.34 (t, J = 55.5 Hz, 1H), 7.26 (br s, 1H), 6.78 (br d, J = 11.5 Hz, 1H), 6.72 (d, J = 13.8 Hz, 1H), 4.70 (br s, 1H), 4.53-4.44 (m, 2H), 4.30-4.25 (m, 1H), 4.11-4.11 (m, 1H), 4.15-4.05 (m, 1H), 3.17 (br dd, J = 1.5, 4.5 Hz, 2H), 2.94 (br s, 2H), 2.73 (br d, J = 3.8 Hz, 4H), 2.29 (br s, 4H), 2.04 (d, J = 16.8 Hz, 3H), 1.92-1.65 (m, 5H), 1.52 (br s, 1H). |
| 407 | 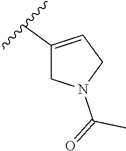 [SMILES: *C1=CCN(C(C)=O)C1] | Isopropyl | N-((R)-1-(7-(1-acetyl- 2,5-dihydro-1H-pyrrol-3- yl)-4-aminopyrrolo[2,1- f][1,2,4]triazin-5- yl)piperidin-3-yl)-5- (difluoromethyl)-3-(1- isopropylpyrrolidin-3- yl)thiophene-2- carboxamide | 613.3 | E: 1.25 F: 1.02 | (400 MHz, DMSO-d₆) δ ppm 8.53 (br d, J = 5.8 Hz, 1H), 8.42 (br s, 1H), 7.88 (s, 1H), 7.50 (br s, 1H), 7.30 (t, J = 53.3 Hz, 1H), 6.93 (br s, 1H), 6.78 (brd, J = 11.0 Hz, 1H), 6.72 (d, J = 12.3 Hz, 1H), 4.70 (br d, J = 2.3 Hz, 1H), 4.49 (br d, J = 10.8 Hz, 2H), 4.27 (br s, 1H), 4.18-4.09 (m, 2H), 3.80 (br s, 2H), 3.20-3.14 (m, 2H), 2.90 (br s, 2H), 2.79-2.69 (m, 2H), 2.42 (s, 1H), 2.17 (br s, 1H), 2.04 (d, J = 16.5 Hz, 3H), 1.88 (br s, 2H), 1.72 (br s, 2H), 1.58-1.45 (m, 1H), 1.02 (br S, 6H) |

### Example 408

### Preparation of methyl (R)-3-(4-amino-5-(3-(3-(2-(diethylamino)ethoxy)-5-(difluoromethyl)thiophene-2-carboxamido)piperidin-1-yl)pyrrolo[2,1-f][1,2,4]triazin-7-yl)-2,5-dihydro-1H-pyrrole-1-carboxylate

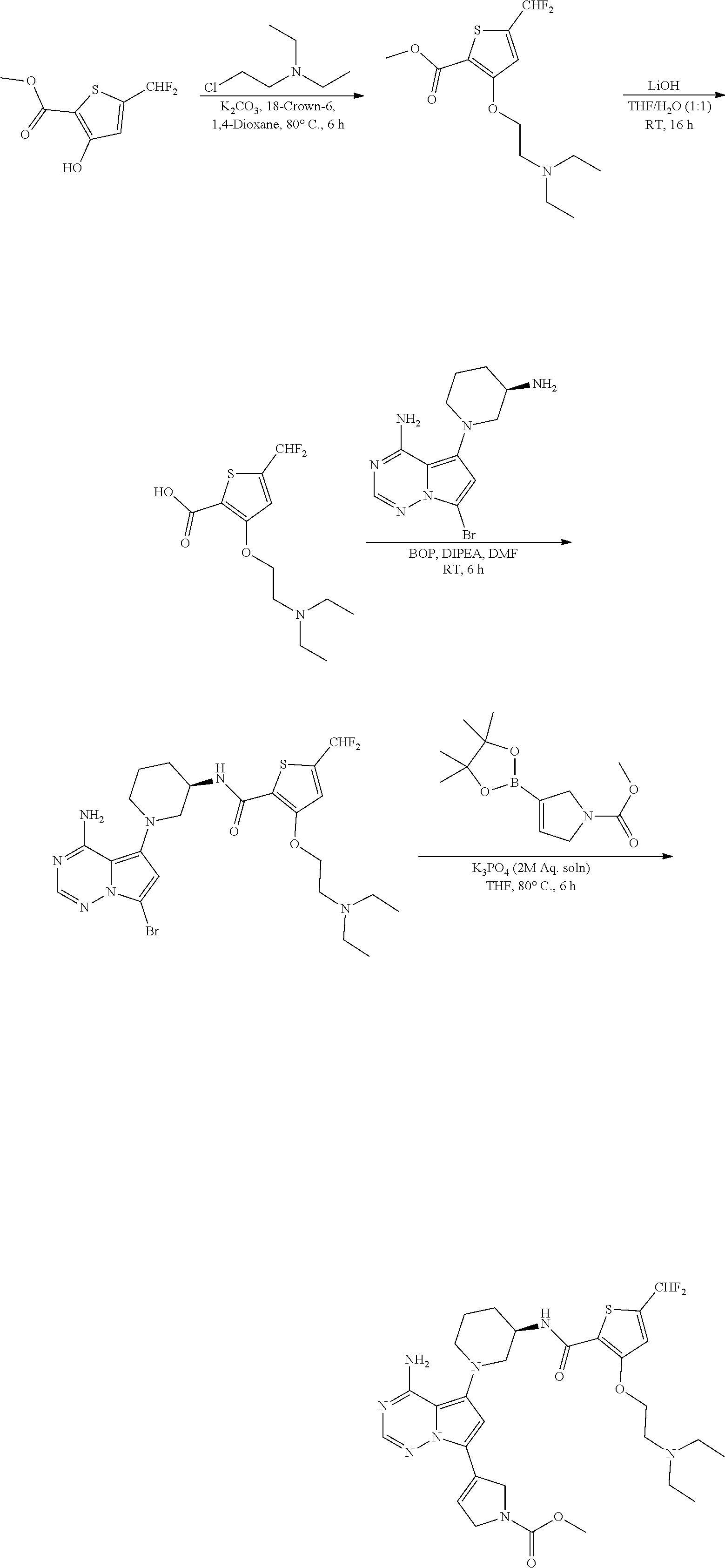

### Example 408A. Methyl 3-(2-(diethylamino)ethoxy)-5-(difluoromethyl)thiophene-2-carboxylate

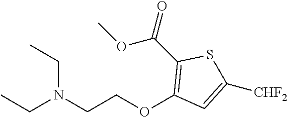 [SMILES: CCN(CC)CCOc1cc(C(F)F)sc1C(=O)OC]

To a mixture of methyl 5-(difluoromethyl)-3-hydroxythiophene-2-carboxylate (250 mg, 1.201 mmol) and K₂CO₃ (498 mg, 3.60 mmol) in 1,4-dioxane (5 mL), was added 2-chloro-N,N-diethylethan-1-amine (163 mg, 1.201 mmol) and 18-crown-6 (31.7 mg, 0.120 mmol). The mixture was stirred at 80° C. for 16 h. After cooling, reaction mixture was diluted with brine and ethyl acetate. Organic layer was separated, dried over anhydrous sodium sulfate and concentrated to get Example 408A (450 mg, 93% yield) as a colorless oil. MS: [M+H]⁺=308.3.

### Example 408B. 3-(2-(diethylamino) ethoxy)-5-(difluoromethyl) thiophene-2-carboxylic acid

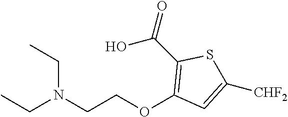 [SMILES: CCN(CC)CCOc1cc(C(F)F)sc1C(=O)O]

Analogous to the procedure for the preparation of Example 276B, reaction of Example 408A (350 mg, 1.139 mmol) and LiOH (82 mg, 3.42 mmol) afforded Example 408B (330 mg, 79% yield) as an off-white solid. MS: [M+H]⁺=294.3.

### Example 408C. (R)—N-(1-(4-amino-7-bromopyrrolo[2, 1-f][1,2,4]triazin-5-yl)piperidin-3-yl)-3-(2-(diethylamino)ethoxy)-5-(difluoromethyl)thiophene-2-carboxamide

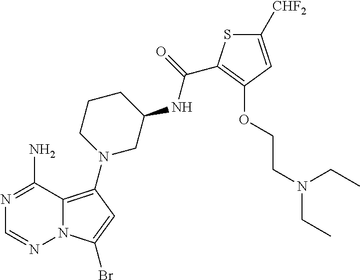 [SMILES: CCN(CC)CCOc1cc(C(F)F)sc1C(=O)N[C@@H]1CCCN(c2cc(Br)n3ncnc(N)c23)C1]

Analogous to the procedure for the preparation of Example 276C, reaction of (R)-5-(3-aminopiperidin-1-yl)-7-bromopyrrolo[2,1-f][1,2,4]triazin-4-amine (728 mg, 2.34 mmol) and Example 408B (350 mg, 1.19 mmol) afforded Example 408C (750 mg, 68% yield) as a brown semisolid. MS: [M+H]⁺=586.3.

### Example 408

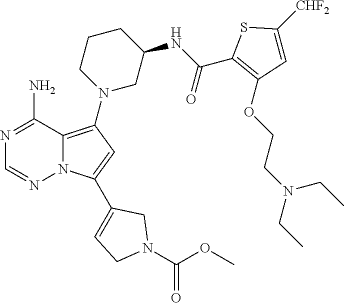 [SMILES: CCN(CC)CCOc1cc(C(F)F)sc1C(=O)N[C@@H]1CCCN(c2cc(C3=CCN(C(=O)OC)C3)n3ncnc(N)c23)C1]

Analogous to the procedure for the preparation of Example 276D, reaction of Example 408C (100 mg, 0.106 mmol) and methyl 3-(4,4,5,5-tetramethyl-1,3,2-dioxaborolan-2-yl)-2,5-dihydro-1H-pyrrole-1-carboxylate (53.5 mg, 0.211 mmol) afforded Example 408 (18.5 mg, 28% yield) as an off-white solid. LC-MS Method E: RT=1.920 min, [M+H]⁺=633.2; LC-MS Method F: RT=1.169 min, [M+H]⁺=633.3; ¹H NMR (400 MHz, DMSO-d₆) δ ppm 9.38 (br s, 1H), 7.91 (s, 1H), 7.56 (d, J=1.7 Hz, 1H), 7.42 (s, 1H), 7.30-7.20 (m, 1H), 7.10 (s, 1H), 6.97 (s, 1H), 6.73 (s, 1H), 4.61-4.49 (m, 3H), 4.34-4.28 (m, 2H), 4.19 (br s, 1H), 3.66 (d, J=4.6 Hz, 4H), 3.57 (br d, J=2.0 Hz, 2H), 3.25-3.17 (m, 4H), 3.00 (br s, 2H), 2.75 (br s, 2H), 1.97-1.90 (m, 2H), 1.61-1.51 (m, 2H), 1.31-1.07 (m, 6H).

The following Example 409 and 410 in Table 29 were prepared using the same procedure as shown in Example 408. Example 408C was coupled with the appropriate boronic acid or boronate ester.

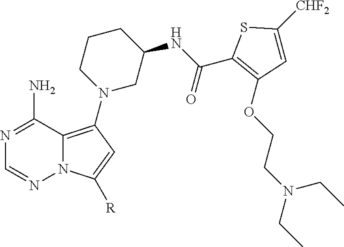

TABLE 29

| | | | | HPLC | |
|---|---|---|---|---|---|
| | | | LCMS | Method, | |
| Example | R | Name | (M + H)⁺ | RT (min.) | ¹H NMR |
| 409 | 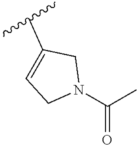 [SMILES: *C1=CCN(C(C)=O)C1] | (R)-N-(1-(7-(1-acetyl- 2,5-dihydro- 1H-pyrrol-3-yl)-4- aminopyrrolo[2,1- f][1,2,4]triazin-5-yl) piperidin-3-yl)-3- (2-(diethylamino) ethoxy)-5- (difluoromethyl) thiophene-2- carboxamide | 617.3 | E: 1.013 F: 1.595 | (400 MHz, DMSO-d₆) δ ppm 8.03 (s, 1H), 7.87 (d, J = 1.2 Hz, 1H), 7.65 (s, 1H), 7.56 (s, 1H), 7.25 (t, J = 54.3 Hz, 1H), 6.80-6.75 (m, 1H),6.71 (d, J = 13.2 Hz, 1H), 4.70 (br s, 1H), 4.55-4.44 (m, 2H), 4.37- 4.23 (m, 4H), 4.19 (m. 2H), 3.17 (m, 2H), 2.95 (m, 2H), 2.85-2.70 (m,4H), 2.03 (d, J = 16.1 Hz, 3H), 1.94-1.69 (m, 3H), 1.55 (s, 1H), 0.98-0.78 (m, 6H). |
| 410 | 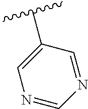 [SMILES: *c1cncnc1] | (R)-N-(1-(4-amino-7- (pyrimidin-5- yl)pyrrolo[2, 1-f] [1,2,4]triazin-5- yl)piperidin-3-yl)-3- (2- (diethylamino) ethoxy)-5- (difluoromethyl) thiophene-2- carboxamide | 586.2 | E: 0.982 F: 1.642 | (400 MHz, DMSO-d₆) δ ppm 9.47 (s, 1H), 9.38 (br s, 1H), 9.11 (s, 1H), 7.95 (s, 1H), 7.56 (s, 1H), 7.28 (s, 1H), 7.23 (s, 1H), 7.10 (s, 1H), 6.97 (s, 1H), 4.60-4.55 (m, 2H), 4.21 (br s, 1H), 3.59-3.55 (m, 3H), 3.27-3.16 (m, 4H), 3.06 (m, 2H), 2.80 (m, 2H), 1.96-1.80 (m, 3H), 1.24-1.11 (m, 6H) |

### Example 411

### Preparation of methyl (R)-3-(5-(3-(3-((1-acetylpiperidin-4-yl)oxy)-5-(difluoromethyl)thiophene-2-carboxamido)piperidin-1-yl)-4-aminopyrrolo[2,1-f][1,2,4]triazin-7-yl)-2,5-dihydro-1H-pyrrole-1-carboxylate

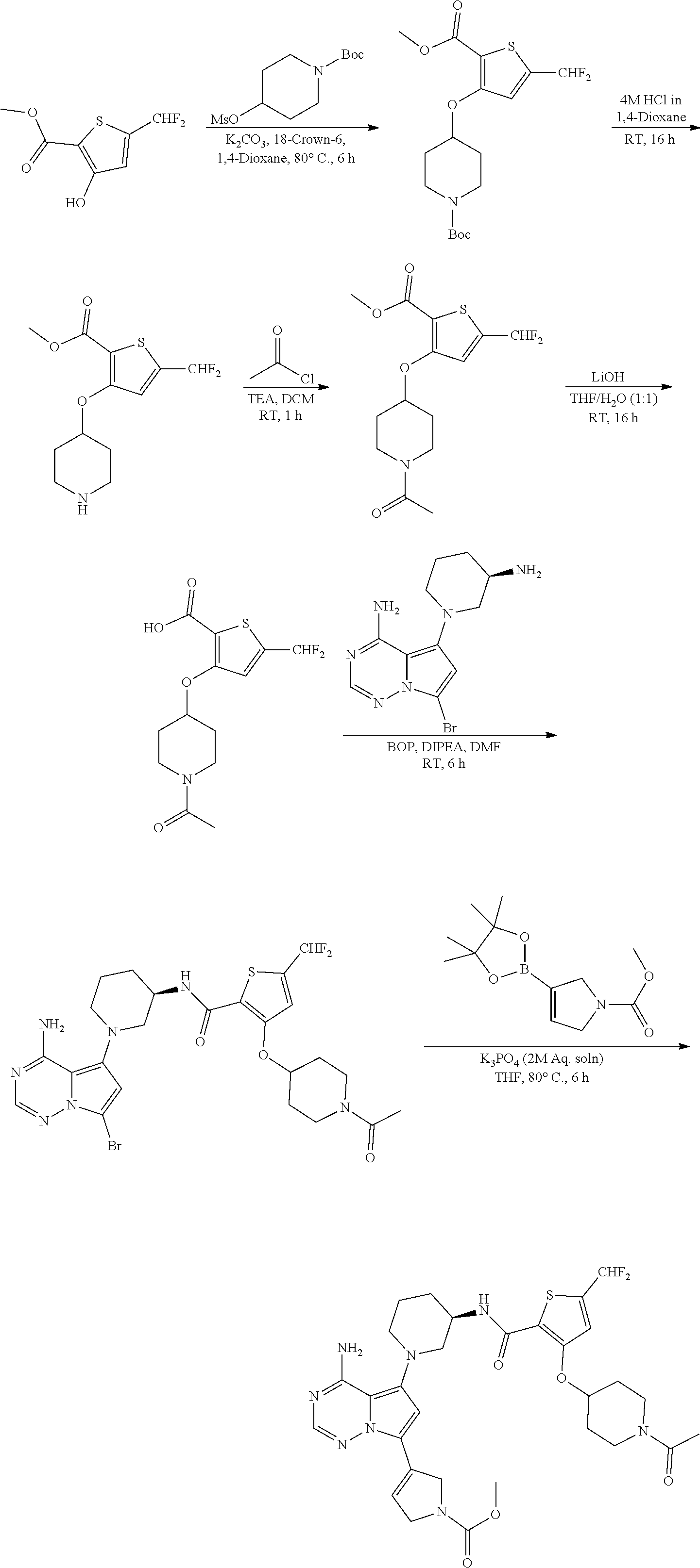

### Example 411A. tert-butyl 4-((5-(difluoromethyl)-2-(methoxycarbonyl) thiophen-3-yl)oxy)piperidine-1-carboxylate

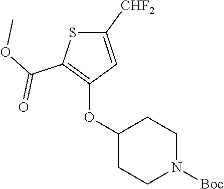 [SMILES: COC(=O)c1sc(C(F)F)cc1OC1CCN(C(=O)OC(C)(C)C)CC1]

To a mixture of methyl 5-(difluoromethyl)-3-hydroxythiophene-2-carboxylate (1 g, 4.80 mmol) and K₂CO₃ (1.33 g, 9.61 mmol) in 1,4-dioxane (30 mL), was added tert-butyl 4-((methylsulfonyl) oxy)piperidine-1-carboxylate (1.74 g, 6.24 mmol) and 18-crown-6 (0.127 g, 0.480 mmol). The mixture was heated at 80° C. for 6 h. After cooling, reaction mixture was diluted with brine water and ethyl acetate. Organic layer was separated, dried over anhydrous sodium sulfate and concentrated which afforded Example 411A as an off-white solid. MS: [M+H]⁺=392.2; ¹H NMR (400 MHz, DMSO-d₆) δ ppm 7.59 (s, 1H), 7.46-7.09 (m, 1H), 4.71 (br s, 1H), 3.78 (s, 3H), 3.61-3.47 (m, 2H), 3.39-3.27 (m, 2H), 1.90-1.80 (m, 2H), 1.69-1.55 (m, 2H), 1.41 (s, 9H).

### Example 411B. Methyl 5-(difluoromethyl)-3-(piperidin-4-yloxy) thiophene-2-carboxylate

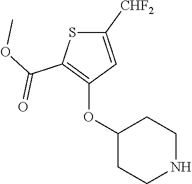 [SMILES: COC(=O)c1sc(C(F)F)cc1OC1CCNCC1]

Example 411A (1.2 g, 3.07 mmol) was treated with 4N HCl in 1,4-dioxane (2.3 mL, 9.20 mmol) which afforded Example 411B (800 mg, 90% yield) as an off-white solid. MS: [M+H]⁺=292.2; ¹H NMR (400 MHz, DMSO-d₆) δ ppm 9.24-9.02 (m, 1H), 7.62 (s, 1H), 7.46-7.09 (m, 1H), 4.83 (td, J=3.0, 6.0 Hz, 1H), 3.80 (s, 3H), 3.25-3.02 (m, 4H), 2.16-1.83 (m, 4H).

### Example 411C. Methyl 3-((1-acetylpiperidin-4-yl) oxy)-5-(difluoromethyl) thiophene-2-carboxylate

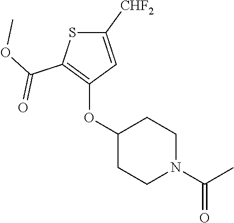 [SMILES: COC(=O)c1sc(C(F)F)cc1OC1CCN(C(C)=O)CC1]

To an ice cold mixture of Example 411B (300 mg, 1.03 mmol) and TEA (0.43 mL, 3.09 mmol) in chloroform (5 mL), was added acetyl chloride (0.095 mL, 1.34 mmol). It was stirred at rt for 1 h, diluted with ethyl acetate (30 mL) and washed with water. Organic layer was separated, dried over anhydrous sodium sulfate and concentrated to afford Example 411C (400 mg, 68% yield) as a brown semi solid. MS: [M+H]⁺=334.2.

### Example 411D. 3-((1-acetylpiperidin-4-yl) oxy)-5-(difluoromethyl) thiophene-2-carboxylic acid lithium salt

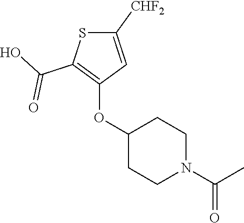 [SMILES: CC(=O)N1CCC(Oc2cc(C(F)F)sc2C(=O)O)CC1]

Analogous to the procedure for the preparation of Example 276B, reaction of Example 411C (600 mg, 1.39 mmol) and LiOH (66.4 mg, 2.77 mmol) afforded Example 411D (450 mg, 84% yield) as an off-white solid. MS: [M+H]⁺=320.2.

### Example 411E. (R)-3-((1-acetylpiperidin-4-yl) oxy)-N-(1-(4-amino-7-bromopyrrolo [2,1-f][1,2,4]triazin-5-yl)piperidin-3-yl)-5-(difluoromethyl)thiophene-2-carboxamide

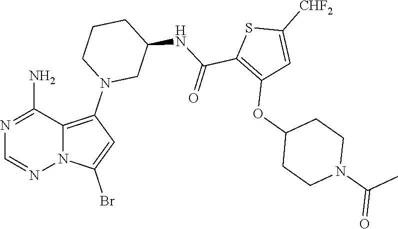 [SMILES: CC(=O)N1CCC(Oc2cc(C(F)F)sc2C(=O)N[C@@H]2CCCN(c3cc(Br)n4ncnc(N)c34)C2)CC1]

Analogous to the procedure for the preparation of Example 276C, reaction of (R)-5-(3-aminopiperidin-1-yl)-7-bromopyrrolo[2,1-f][1,2,4]triazin-4-amine (765 mg, 2.46 mmol) and Example 411D (400 mg, 1.23 mmol) afforded Example 411E (950 mg, 58% yield) as a brown semisolid. MS: [M+H]⁺=612.3.

### Example 411

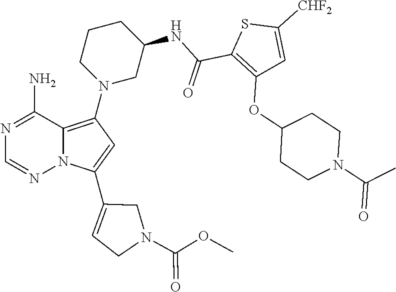 [SMILES: COC(=O)N1CC=C(c2cc(N3CCC[C@@H](NC(=O)c4sc(C(F)F)cc4OC4CCN(C(C)=O)CC4)C3)c3c(N)ncnn23)C1]

Analogous to the procedure for the preparation of Example 276D, reaction of Example 411E (100 mg, 0.073 mmol) and methyl 3-(4,4,5,5-tetramethyl-1,3,2-dioxaborolan-2-yl)-2,5-dihydro-1H-pyrrole-1-carboxylate (37.2 mg, 0.147 mmol) afforded Example 411 (17.9 mg, 36% yield) as an off-white solid. LC-MS Method E: RT=1.630 min, [M+H]⁺=659.2; LC-MS Method F: RT=1.345 min, [M+H]⁺=659.3; ¹H NMR (400 MHz, DMSO-d₆) δ ppm 8.05 (br s, 1H), 7.86 (s, 1H), 7.62 (s, 1H), 7.47 (br s, 1H, 7.25 (s, 1H), 6.76-6.68 (m, 2H), 4.81-4.76 (m, 1H), 4.60-4.49 (m, 2H), 4.34-4.26 (m, 2H), 4.17 (br dd, J=1.3, 3.8 Hz, 1H), 3.91 (s, 3H), 3.83 (br s, 2H), 3.66 (d, J=4.2 Hz, 2H), 3.22-3.10 (m, 3H), 2.97-2.72 (m, 3H), 2.07-1.89 (m, 4H), 1.84-1.74 (m, 2H), 1.71-1.50 (m, 3H).

The following examples in Table 30 were prepared using the same procedure as shown in Example 411. Example 411E was coupled with the appropriate boronic acid or boronate ester.

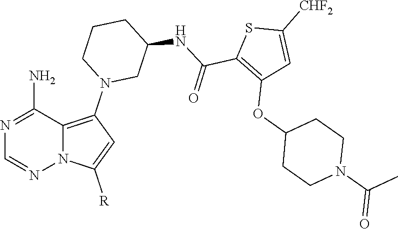

TABLE 30

| | | | | HPLC | |
|---|---|---|---|---|---|
| | | | LCMS | Method, | |
| Example | R | Name | (M + H)⁺ | RT (min.) | ¹H NMR |
| 412 | 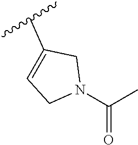 [SMILES: *C1=CCN(C(C)=O)C1] | (R)-N-(1-(7-(1-acetyl-2,5-dihydro- 1H-pyrrol-3-yl)-4- aminopyrrolo[2,1-f][1,2,4]triazin-5- yl)piperidin-3-yl)-3-((1- acetylpiperidin-4-yl)oxy)-5- (difluoromethyl)thiophene-2- carboxamide | 643.3 | E: 1.204 F: 1.427 | (400 MHz, DMSO-d₆) δ ppm 7.87 (d, J = 2.5 Hz, 1H), 7.62 (s, 1H), 7.54-7.43 (m, 1H), 7.24 (t, J = 53.8 Hz, 1H), 6.83-6.61 (m, 2H), 4.78 (br d, J = 2.8 Hz, 1H), 4.69 (br s, 1H), 4.48 (br d, J = 8.8 Hz, 2H), 4.27 (br s, 1H), 4.18 (br d. J = 3.0 Hz, 1H), 3.96-3.79 (m, 3H), 3.70-3.59 (m, 1H), 3.28- 3.09 (m, 3H), 3.05-2.69 (m, 4H), 2.10-1.77 (m, 8H), 1.73-1.43 (m, 3H) |
| 413 | 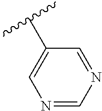 [SMILES: *c1cncnc1] | (R)-3-((1-acetylpiperidin-4-yl)oxy)- N-(1-(4-amino-7-(pyrimidin-5- yl)pyrrolo[2,1-f][1,2,4]triazin-5- yl)piperidin-3-yl)-5- (difluoromethyl)thiophene-2- carboxamide | 612.2 | E: 1.239 F: 1.417 | (400 MHz, DMSO-d₆) δ ppm 9.46 (s, 2H), 9.08 (s, 1H), 8.14 (s, 1H), 7.91 (s, 1H), 7.62 (s, 1H), 7.48 (br d, J = 3.0 Hz, 1H), 7.29 (s, 1H), 7.24 (t, J = 73.0 Hz, 1H), 6.82 (s, 1H), 4.83-4.72 (m, 1H), 4.24-4.16 (m, 1H), 3.90 (s, 3H), 3.63 (br s, 1H), 3.20 (br s, 2H), 3.03-2.96 (m, 1H), 2.85 (br s, 2H), 2.06- 1.77 (m, 6 H), 1.71-1.51 (m, 4H) |
| 414 | 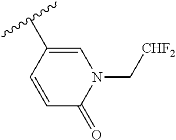 [SMILES: *c1ccc(=O)n(CC(F)F)c1] | (R)-3-((1-acetylpiperidin-4-yl)oxy)- N-(1-(4-amino-7-(1-(2,2- difluoroethyl)-6-oxo-1,6- dihydropyridin-3-yl)pyrrolo[2,1- f][1,2,4]triazin-5-yl)piperidin-3-yl)- 5-(difluoromethyl)thiophene-2- carboxamide | 691.2 | E: 1.345 F: 1.525 | (400 MHz, DMSO-d₆) δ ppm 8.49 (d, J = 2.3 Hz, 1H), 8.46 (s, 1H), 8.11 (dd, J = 1.96, 9.6 Hz, 1H), 7.81 (s, 1H), 7.62 (s, 1H), 7.50 (br s, 1H), 7.24 (t, J = 56.3 Hz, 1H), 6.89 (s, 1H), 6.56 (d, J = 9.5 Hz, 1H), 6.52-6.18 (m, 1H), 4.83-4.74 (m, 1H), 4.50-4.39 (m, 2H), 4.18 (br dd, J = 2.4, 4.9 Hz, 1H), 3.77 (s, 1H), 3.62 (br d, J = 4.0 Hz, 1H), 3.19 (dtd, J = 2.0, 4.6, 7.0 Hz, 2H), 3.02-2.73 (m, 3H), 2.10-1.99 (m, 4H), 1.98-1.87 (m, 4H), 1.86-1.77 (m, 2H), 1.70-1.47 (m, 2H) |
| 415 | 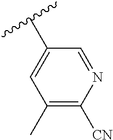 [SMILES: *c1cnc(C#N)c(C)c1] | (R)-3-((1-acetylpiperidin-4-yl)oxy)- N-(1-(4-amino-7-(6-cyano-5- methylpyridin-3-yl)pyrrolo[2,1- f][1,2,4]triazin-5-yl)piperidin-3-yl)- 5-(difluoromethyl)thiophene-2- carboxamide | 650.3 | E: 1.546 F: 1.778 | (400 MHz, DMSO-d₆) δ ppm 9.31 (d, J = 1.5 Hz, 1H), 8.66 (d, J = 1.0 Hz, 1H), 8.18 (br s, 1H), 7.96 (s, 1H), 7.63 (s, 1H), 7.49 (br s, 1H), 7.36 (s, 1H), 7.25 (t, J = 55.5 Hz, 1H), 6.86 (br s, 1H), 4.79 (dt, J = 3.5, 7.8 Hz, 1H), 4.29-4.14 (m, 1H), 3.94-3.75 (m, 1H), 3.73-3.59 (m, 1H), 3.27-3.16 (m, 2H), 3.06-2.72 (m, 3H), 2.54 (s, 3H), 2.07- 1.78 (m, 9H), 1.75-1.46 (m, 3H) |
| 416 | 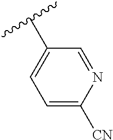 [SMILES: *c1ccc(C#N)nc1] | (R)-3-((1-acetylpiperidin-4-yl)oxy)- N-(1-(4-amino-7-(6-cyanopyridin-3- yl)pyrrolo[2, 1-f][1,2,4]triazin-5- yl)piperidin-3-yl)-5- (difluoromethyl)thiophene-2- carboxamide | 636.2 | E: 1.470 F: 1.703 | (400 MHz, DMSO-d₆) δ ppm 9.43 (d, J = 2.0 Hz, 1H), 8.80 (dd, J = 2.0, 8.3 Hz, 1H), 8.24 (br s, 1H), 8.09 (d, J = 8.5 Hz, 1H), 7.95 (s, 1H), 7.62 (s, 1H), 7.49 (br s, 1H), 7.41-7.06 (m, 2H), 6.96 (s, 1H), 4.83-4.73 (m, 1H), 4.24-4.13 (m, 1H), 3.91-3.76 (m, 1H), 3.69-3.60 (m, 1H), 3.17 (s, 2H), 3.05- 2.73 (m, 4H), 2.06-1.77 (m, 6H), 1.72-1.47 (m, 3H), 1.16 (t, J = 7.3 Hz, 2H) |

### Example 418

### Preparation of Methyl (R)-3-(4-amino-5-(3-(5-chloro-3-(2-oxo-2-(pyrrolidin-1-yl)ethoxy)thiophene-2-carboxamido)piperidin-1-yl)pyrrolo[2,1-f][1,2,4]triazin-7-yl)-2,5-dihydro-1H-pyrrole-1-carboxylate

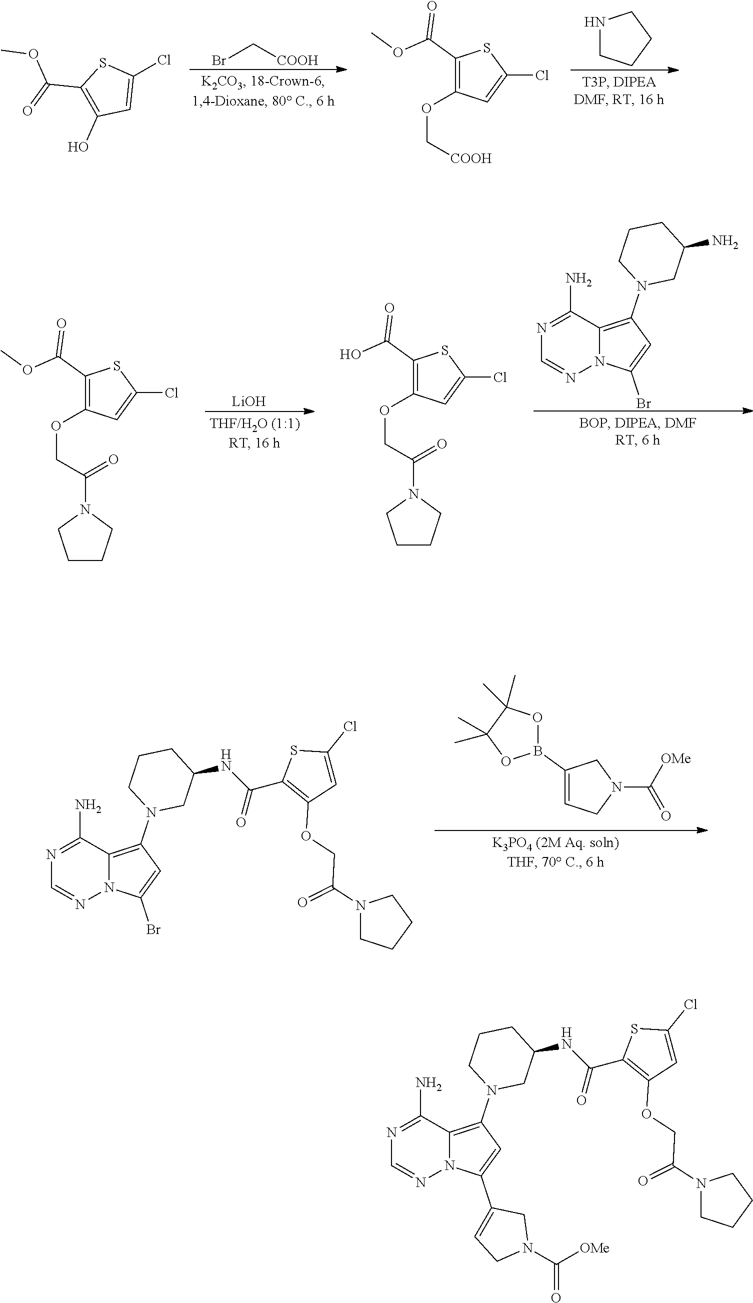

### Example 418A. 2-((5-chloro-2-(methoxycarbonyl)thiophen-3-yl)oxy)acetic acid

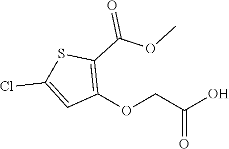

a mixture or methyl 3-chloro-3-hydroxythiophene-2-carboxylate (500 mg, 2.60 mmol) and K₂CO₃ (1.08 g, 7.79 mmol) in 1,4-dioxane (10 mL), was added 2-bromoacetic acid (541 mg, 3.89 mmol) and 18-crown-6 (68.6 mg, 0.260 mmol). The mixture was stirred at 80° C. for 16 h, cooled to rt and acidified to pH-4 using aq. 2N HCl solution. Product was extracted in ethyl acetate (40 mL), dried over anhydrous sodium sulfate and concentrated to afford Example 418A (1.2 g, 86% yield) as an off-white solid. MS: [M+H]⁺=251.1.

### Example 418B. Methyl 5-chloro-3-(2-oxo-2-(pyrrolidin-1-yl) ethoxy) thiophene-2-carboxylate

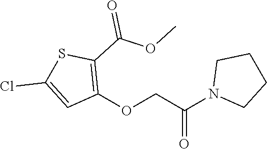 [SMILES: COC(=O)c1sc(Cl)cc1OCC(=O)N1CCCC1]

To a mixture of Example 418A (500 mg, 0.898 mmol), pyrrolidine (0.160 mL, 1.80 mmol) and propylphosphonic anhydride (50% solution in ethyl acetate, 1.59 mL, 2.69 mmol) in DMF (5 mL), was added DIEA (0.47 mL, 2.69 mmol). The mixture was stirred at rt for 6 h followed by the addition of water (40 mL) and ethyl acetate (30 mL). Organic layer was separated, dried over anhydrous sodium sulfate and concentrated to get the crude product. The crude product was purified by silica-gel chromatography using 12% ethyl acetate in petroleum-ether to afford Example 418B (200 mg, 72% yield) as an off-white solid. MS: [M+H]⁺=304.0.

### Example 418C. (R)—N-(1-(4-amino-7-bromopyrrolo [2,1-f][1,2,4]triazin-5-yl)piperidin-3-yl)-5-chloro-3-(2-oxo-2-(pyrrolidin-1-yl)ethoxy)thiophene-2-carboxamide, lithium salt

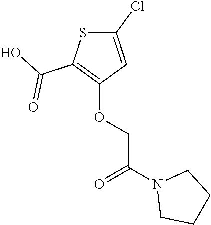 [SMILES: O=C(O)c1sc(Cl)cc1OCC(=O)N1CCCC1]

Analogous to the procedure for the preparation of Example 276B, reaction of Example 418B (200 mg, 0.658 mmol) and LiOH (47.3 mg, 1.98 mmol) afforded Example 418C (180 mg, 77% yield) as an off-white solid. MS: [M+H]⁺=290.2.

### Example 418D. (R)—N-(1-(4-amino-7-bromopyrrolo [2,1-f][1,2,4]triazin-5-yl)piperidin-3-yl)-5-chloro-3-(2-oxo-2-(pyrrolidin-1-yl)ethoxy)thiophene-2-carboxamide

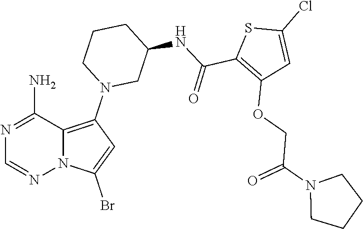 [SMILES: Nc1ncnn2c(Br)cc(N3CCC[C@@H](NC(=O)c4sc(Cl)cc4OCC(=O)N4CCCC4)C3)c12]

Analogous to the procedure for the preparation of Example 276C, reaction of (R)-5-(3-aminopiperidin-1-yl)-7-bromopyrrolo[2,1-f][1,2,4]triazin-4-amine (421 mg, 1.35 mmol) and Example 418C (200 mg, 0.676 mmol) afforded Example 418D (510 mg, 77% yield) as a brownish semisolid. MS: [M+H]⁺=582.2.

### Example 418

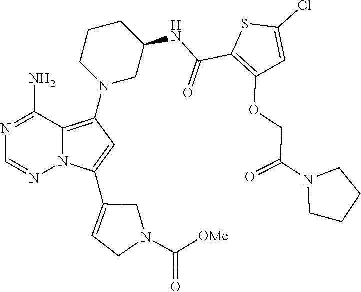 [SMILES: COC(=O)N1CC=C(c2cc(N3CCC[C@@H](NC(=O)c4sc(Cl)cc4OCC(=O)N4CCCC4)C3)c3c(N)ncnn23)C1]

Analogous to the procedure for the preparation of Example 276D, reaction of Example 418D, (65 mg, 0.066 mmol) and methyl 3-(4,4,5,5-tetramethyl-1,3,2-dioxaborolan-2-yl)-2,5-dihydro-1H-pyrrole-1-carboxylate (16.7 mg, 0.066 mmol) afforded Example 418 (7.2 mg, 17% yield). LC-MS Method E: RT=1.504 min, [M+H]⁺=629.3; LC-MS Method F: RT=1.773 min, [M+H]⁺=629.3; ¹H NMR (400 MHz, DMSO-d₆) δ ppm 8.45 (s, 1H), 7.88 (s, 1H), 7.34 (s, 1H), 6.80-6.70 (m, 2H), 4.98 (s, 2H), 4.60-4.47 (m, 2H), 4.30-4.20 (m, 2H), 3.66 (d, J=3.5 Hz, 2H), 3.13-3.08 (m, 4H), 2.97-2.60 (m, 4H), 1.88 (br d, J=6.3 Hz, 2H), 1.81-1.68 (m, 2H), 1.17 (q, J=7.2 Hz, 6H).

The following examples in Table 31 were prepared using the same procedure as shown in Example 418. Example 418D was coupled with the appropriate boronic acid or boronate ester.

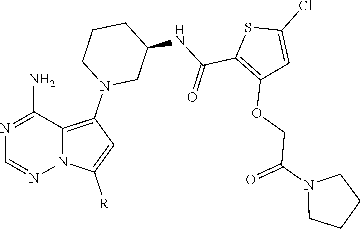

TABLE 31

| | | | | HPLC | |
|---|---|---|---|---|---|
| | | | LCMS | Method, | |
| Example | R | Name | (M + H)⁺ | RT (min.) | ¹H NMR |
| 419 | 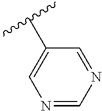 [SMILES: *c1cncnc1] | (R)-N-(1-(4- amino-7- (pyrimidin-5- yl)pyrrolo[2,1- f][1,2,4] triazin-5- yl)piperidin-3- yl)-5- chloro- 3-(2-oxo-2- (pyrrolidin-1- yl)ethoxy) thiophene-2- carboxamide | 582.2 | E: 1.379 F: 1.610 | (400 MHz, DMSO-d6) δ ppm 9.45 (s, 2H), 9.10 (s, 1H), 8.51-8.24 (m, 2H), 7.95 (s, 1H), 7.34 (d, J = 2.0 Hz, 2H), 7.23-6.90 (m, 1H), 4.97 (s, 2H), 4.20 (br s, 1H), 3.37-3.34 (m, 3H), 3.31-3.21 (m, 3H), 3.03-2.79 (m, 3H), 2.01-1.69 (m, 6H), 1.63 (br s, 1H) |
| 420 | 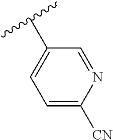 [SMILES: *c1ccc(C#N)nc1] | (R)-N-(1-(4- amino-7-(6- cyanopyridin-3- yl)pyrrolo[2,1- f][1,2,4]triazin-5- yl)piperidin-3- yl)-5-chloro- 3-(2-oxo-2- (pyrrolidin-1- yl)ethoxy) thiophene-2- carboxamide | 607.2 | E: 1.593 F: 1.909 | (400 MHz, DMSO-d6) δ ppm 9.44 (d, J = 2.0 Hz, 1H), 8.81 (dd, J = 2.3, 8.3 Hz, 1H), 8.52-8.41 (m, 1H), 8.09 (d,) = 8.5 Hz, 2H), 7.95 (s, 1H), 7.43 (s, 1H), 6.97 (m, 1H), 4.98 (s, 2H), 4.24-4.14 (m, 1H), 3.22 (m, 3H), 2.94 (m, 3H), 1.98-1.68 (m, 7H), 1.59 (s, 3H). |
| 421 | 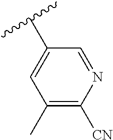 [SMILES: *c1cnc(C#N)c(C)c1] | (R)-N-(1-(4- amino-7-(6- cyano-5- methylpyridin-3- yl)pyrrolo[2,1- f][1,2,4]triazin-5- yl)piperidin-3- yl)-5-chloro- 3-(2-oxo-2- (pyrrolidin-1- yl)ethoxy) thiophene-2- carboxamide | 620.2 | E: 1.687 F: 1.990 | |
| 422 | 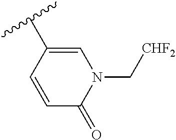 [SMILES: *c1ccc(=O)n(CC(F)F)c1] | (R)-N-(1-(4- amino-7-(1- (2,2- difluoroethyl)- 6-oxo- 1,6- dihydropyridin-3- yl)pyrrolo[2,1- f][1,2,4]triazin-5- yl)piperidin-3-yl)- 5-chloro- 3-(2-oxo-2- (pyrrolidin-1- yl)ethoxy) thiophene-2- carboxamide | 661.2 | E: 1.470 F: 1.681 | |

### Example 423

### Preparation of methyl (R)-3-(5-(3-(3-(2-acetamidoethoxy)-5-(difluoromethyl)thiophene-2-carboxamido)piperidin-1-yl)-4-aminopyrrolo[2,1-f][1,2,4]triazin-7-yl)-2,5-dihydro-1H-pyrrole-1-carboxylate

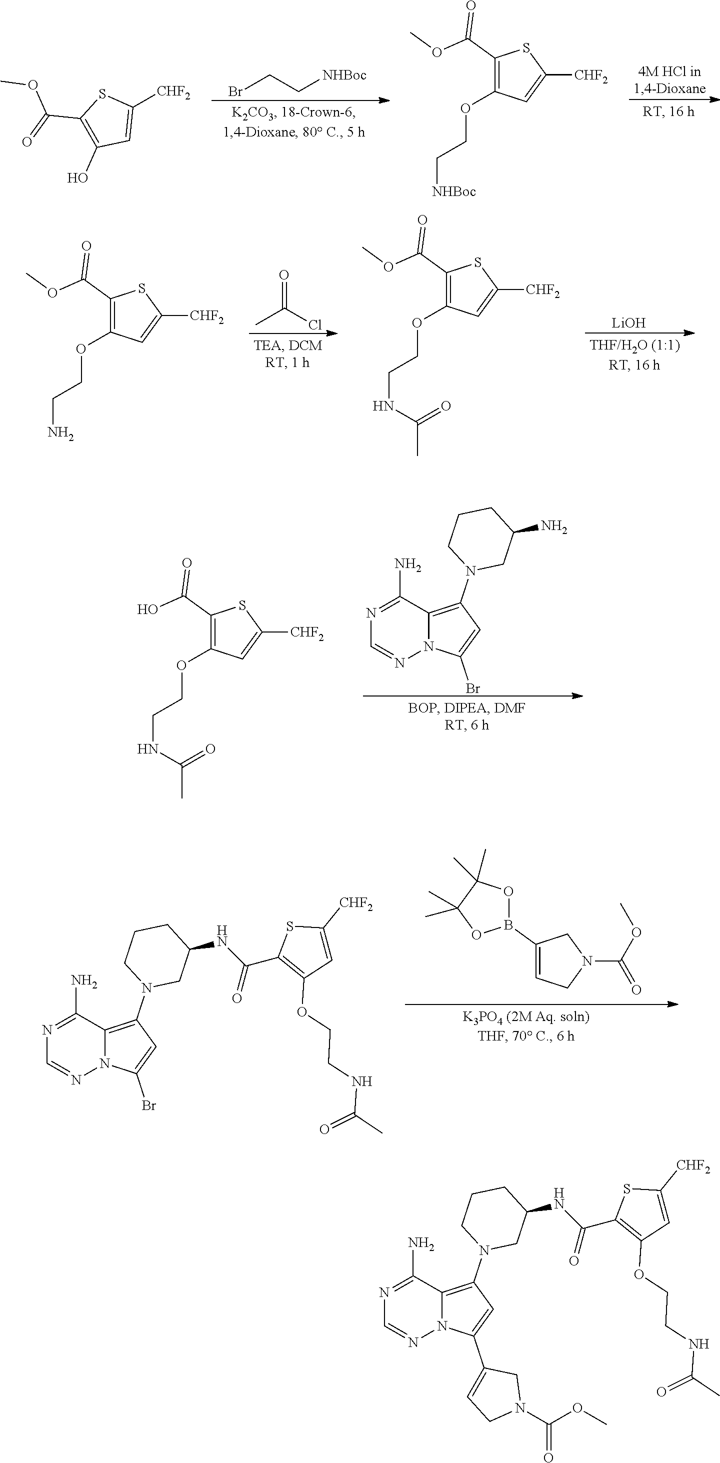

### Example 423A. Methyl 3-(2-((tert-butoxycarbonyl) amino) ethoxy)-5-(difluoromethyl) thiophene-2-carboxylate

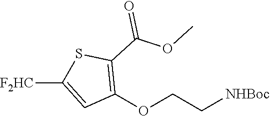 [SMILES: COC(=O)c1sc(C(F)F)cc1OCCNC(=O)OC(C)(C)C]

To a mixture of methyl 5-(difluoromethyl)-3-hydroxythiophene-2-carboxylate (2 g, 9.61 mmol) and K₂CO₃ (3.98 g, 28.8 mmol) in 1,4-dioxane (50 mL), was added tert-butyl (2-bromoethyl)carbamate (2.153 g, 9.61 mmol) and 18-crown-6 (0.254 g, 0.961 mmol). The mixture was stirred at 80° C. for 16 h followed by the addition of brine (100 mL) and ethyl acetate (60 mL). Organic layer was separated, dried over anhydrous sodium sulfate and concentrated to get the crude product. The crude product was purified on silica-gel chromatography using 3% Methanol in Chloroform to afford Example 423A (3 g, 89% yield) as an off-white solid. MS: [M+Na]⁺=374.0.

### Example 423B. Methyl 3-(2-aminoethoxy)-5-(difluoromethyl) thiophene-2-carboxylate

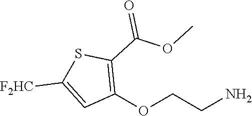 [SMILES: COC(=O)c1sc(C(F)F)cc1OCCN]

To an ice cold solution of Example 423A (2 g, 5.69 mmol) in 1,4-dioxane (30 mL), was added 4M HCl in 1,4-dioxane (7.12 mL, 28.5 mmol). The mixture was stirred at room temperature for 16 h and concentrated to afford Example 423B (1.4 g, 73% yield) as an off-white solid. MS: [M+H]⁺=252.2.

### Example 423C. 3-(2-acetamidoethoxy)-5-(difluoromethyl) thiophene-2-carboxylic acid

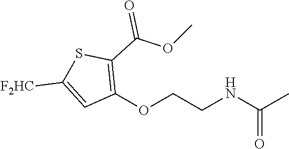 [SMILES: COC(=O)c1sc(C(F)F)cc1OCCNC(C)=O]

To a mixture of Example 423B (250 mg, 0.995 mmol) and TEA (0.416 mL, 2.99 mmol) in dichloromethane (2 mL), was added acetyl chloride (0.071 mL, 0.995 mmol). The mixture was stirred at rt for 1 h and quenched with ice water and extracted with DCM (40 mL). Organic layer was separated, dried over anhydrous sodium sulfate and concentrated to afford Example 423C (290 mg, 81% yield) as a colorless oil. MS: [M+H]⁺=294.2.

### Example 423D. 3-(2-acetamidoethoxy)-5-(difluoromethyl) thiophene-2-carboxylic acid

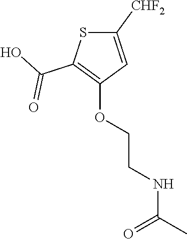 [SMILES: CC(=O)NCCOc1cc(C(F)F)sc1C(=O)O]

Analogous to the procedure for the preparation of Example 276B, reaction of Example 423C (300 mg, 0.829 mmol) and LiOH (59.5 mg, 2.49 mmol) afforded Example 423D (230 mg, 84% yield) as an off-white solid. MS: [M+H]⁺=280.2.

### Example 423E. (R)-3-(2-acetamidoethoxy)-N-(1-(4-amino-7-bromopyrrolo [2,1-f][1,2,4]triazin-5-yl)piperidin-3-yl)-5-(difluoromethyl)thiophene-2-carboxamide

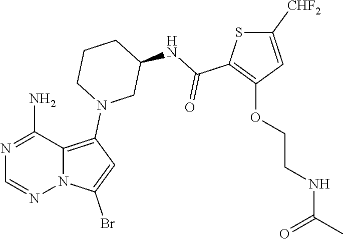 [SMILES: CC(=O)NCCOc1cc(C(F)F)sc1C(=O)N[C@@H]1CCCN(c2cc(Br)n3ncnc(N)c23)C1]

Analogous to the procedure for the preparation of Example 276C, reaction of (R)-5-(3-aminopiperidin-1-yl)-7-bromopyrrolo[2,1-f][1,2,4]triazin-4-amine (780 mg, 2.51 mmol) and Example 423D (350 mg, 1.25 mmol) afforded Example 423E, (670 mg, 58% yield) as a brown semisolid. MS: [M+H]⁺=572.2.

### Example 423

### Methyl (R)-3-(5-(3-(3-(2-acetamidoethoxy)-5-(difluoromethyl)thiophene-2-carboxamido)piperidin-1-yl)-4-aminopyrrolo[2,1-f][1,2,4]triazin-7-yl)-2,5-dihydro-1H-pyrrole-1-carboxylate

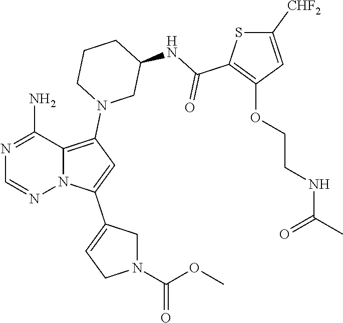 [SMILES: COC(=O)N1CC=C(c2cc(N3CCC[C@@H](NC(=O)c4sc(C(F)F)cc4OCCNC(C)=O)C3)c3c(N)ncnn23)C1]

Analogous to the procedure for the preparation of Example 276D, reaction of Example 423E (100 mg, 0.105 mmol) and methyl 3-(4,4,5,5-tetramethyl-1,3,2-dioxaborolan-2-yl)-2,5-dihydro-1H-pyrrole-1-carboxylate (26.5 mg, 0.105 mmol) afforded Example 423 (27.1 mg, 40% yield) as an off-white solid. LC-MS Method E: RT=1.573 min, [M+H]⁺=619.2; LC-MS Method F: RT=1.325 min, [M+H]⁺=619.2; ¹H NMR (400 MHz, DMSO-d₆) δ ppm 8.15-8.07 (m, 1H), 7.85 (s, 1H), 7.50 (s, 2H), 7.24 (t, J=55.3 Hz, 1H), 6.76-6.71 (m, 2H), 4.58-4.48 (m, 2H), 4.34-4.14 (m, 5H), 3.65 (d, J=4.3 Hz, 3H), 3.48-3.41 (m, 2H), 3.15-3.07 (m, 1H), 3.04-2.95 (m, 1H), 2.87-2.69 (m, 1H), 1.92-1.76 (m, 7H), 1.60-1.50 (m, 2H).

The following examples in Table 32 were prepared using the same procedure as shown in Example 423. Example 423E was coupled with the appropriate boronic acid or boronate ester.

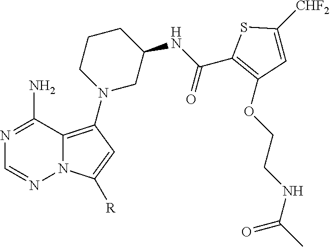

TABLE 32

| | | | | HPLC | |
|---|---|---|---|---|---|
| Ex- | | | LCMS | Method, | |
| ample | R | Name | (M + H)⁺ | RT (min.) | ¹H NMR |
| 424 | 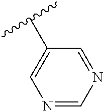 [SMILES: *c1cncnc1] | (R)-3-(2-acetamidocthoxy)-N- (1-(4-amino-7-(pyrimidin-5- yl)pyrrolo[2,1-f][1,2,4]triazin- 5-yl)piperidin-3-yl)-5- (difluoromethyl)thiophene-2- carboxamide | 572.2 | E: 1.075 F: 1.318 | (400 MHz, DMSO-d₆) δ ppm 9.47 (s, 2H), 9.08 (s, 1H), 8.12 (s, 1H), 7.91 (s, 1H), 7.50 (s, 2H), 7.38-7.11 (m, 2H), 4.26 (br t, J = 5.0 Hz, 3H), 3.49-3.20 (m, 4H), 3.19-3.15 (m, 1H), 3.03 (br s, 1H), 2.79 (s, 2H), 1.82 (s, 6H), 1.69 (br s, 1H) |
| 425 | 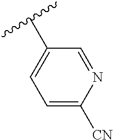 [SMILES: *c1ccc(C#N)nc1] | (R)-3-(2-acetamidocthoxy)-N- (1-(4-amino-7-(6- cyanopyridin-3- yl)pyrrolo[2,1-f][1,2,4]triazin- 5-yl)piperidin-3-yl)-5- (difluoromethyl)thiophene-2- carboxamide | 597.3 | E: 1.337 F: 1.601 | (400 MHz, DMSO-d₆) δ ppm 9.43 (d, J = 2.0 Hz, 1H), 8.81 (dd, J = 2.0, 8.5 Hz, 1H), 8.22- 8.05 (m, 3H), 7.95 (s, 1H), 7.54-7.39 (m, 2H), 7.38-7.08 (m, 1H), 6.93 (s, 1H), 4.29-4.20 (m, 4H), 3.49-3.46 (m, 2H), 3.19-3.16 (m, 1H), 3.06-3.01 (m, 1H), 2.86 (s, 2H), 1.92-1.77 (m, 6H), 1.70 (s, 1H) |
| 426 | 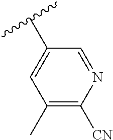 [SMILES: *c1cnc(C#N)c(C)c1] | (R)-3-(2-acetamidoethoxy)-N- (1-(4-amino-7-(6-cyano-5- methylpyridin-3- yl)pyrrolo[2,1-f][1,2,4]triazin- 5-yl)piperidin-3-yl)-5- (difluoromethyl)thiophene-2- carboxamide | 610.3 | E: 1.446 F: 1.694 | (400 MHz, DMSO-d₆) δ ppm 9.29 (d, J = 1.8 Hz, 1H), 8.64 (d, J = 1.5 Hz, 1H), 8.25 (br s, 1H), 8.13 (br t, J = 5.1 Hz, 1H), 7.96 (s, 1H), 7.50 (s, 2H), 7.37 (s, 1H), 7.28-6,91 (m, 1H), 4.31-4.17 (m, 3H), 3.52- 3.46 (m, 2H), 3.22-3.15 (m, 1H), 3.09-3.00 (m, 1H), 2.97- 2.68 (m, 3H), 2.53 (s, 3H), 1.82 (s, 7H) |
| 427 | 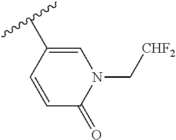 [SMILES: *c1ccc(=O)n(CC(F)F)c1] | (R)-3-(2-acetamidoethoxy)-N- (1-(4-amino-7-(1-(2,2- difluoroethyl)-6-oxo-1,6- dihydropyridin-3- yl)pyrrolo[2,1-f][1,2,4]triazin- 5-yl)piperidin-3-yl)-5- (difluoromethyl)thiophene-2- carboxamide | 651.2 | E: 1.224 F: 1.419 | (400 MHz, DMSO-d₆) δ ppm 8.49 (d, J = 2.5 Hz, 1H), 8.16- 8.04 (m, 2H), 7.92 (s, 1H), 7.51-7.42 (m, 1H), 7.39-7.05 (m, 2H), 6.97 (s, 1H), 6.59 (d, J = 9.8 Hz, 1H), 6.51-6.17 (m, 1H), 4.54-4.39 (m, 2H), 4.24 (br t, J = 5.0 Hz, 3H), 3.65-3.42 (m, 3H), 3.20-3.17 (m, 1H), 3.09-3.05 (m, 1H), 2.89-2.83 (m, 1H), 2.80-2.71 (m, 1H), 1.94-1.78 (m, 7H), 1.74-1.65 (m, 1H) |

### Example 428

### Preparation of methyl (R)-3-(4-amino-5-(3-(5-(difluoromethyl)-3-(2-((methoxycarbonyl)amino)ethoxy)thiophene-2-carboxamido)piperidin-1-yl)pyrrolo[2,1-f][1,2,4]triazin-7-yl)-2,5-dihydro-1H-pyrrole-1-carboxylate

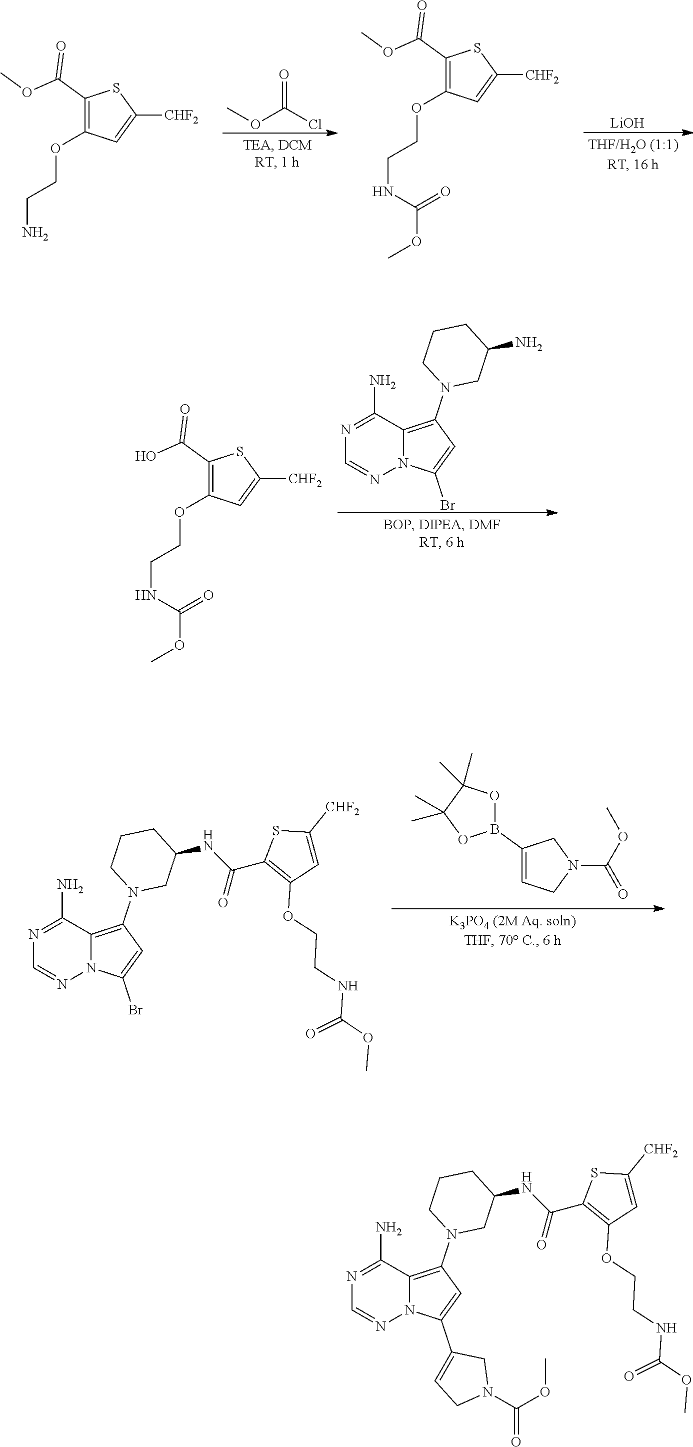

### Example 428A. Methyl 5-(difluoromethyl)-3-(2-((methoxycarbonyl) amino)ethoxy)thiophene-2-carboxylate

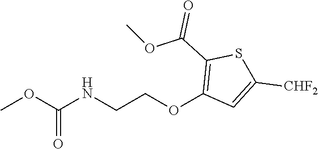 [SMILES: COC(=O)NCCOc1cc(C(F)F)sc1C(=O)OC]

Analogous to the procedure for the preparation of Example 423C, reaction of Example 423B (250 mg, 0.995 mmol) and methyl carbonochloridate (0.077 mL, 0.995 mmol) afforded Example 428A (300 mg, 64% yield) as a colorless oil. MS: [M+Na]⁺=332.2.

### Example 428B. 5-(difluoromethyl)-3-(2-((methoxycarbonyl) amino) ethoxy) thiophene-2-carboxylic acid

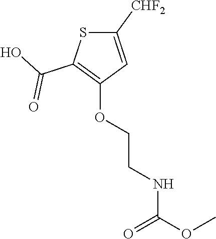 [SMILES: COC(=O)NCCOc1cc(C(F)F)sc1C(=O)O]

Analogous to the procedure for the preparation of Example 276B, reaction of Example 428A (300 mg, 0.630 mmol) and LiOH (45.3 mg, 1.89 mmol) afforded Example 428B (200 mg, 77% yield) as an off-white solid. MS: [M+H]⁺=296.23

### Example 428C. Methyl (R)-(2-((2-((1-(4-amino-7-(6-cyanopyridin-3-yl)pyrrolo[2,1-f][1,2,4]triazin-5-yl)piperidin-3-yl)carbamoyl)-5-(difluoromethyl)thiophen-3-yl)oxy)ethyl)carbamate

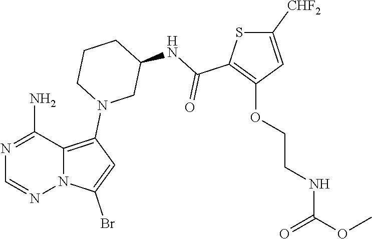 [SMILES: COC(=O)NCCOc1cc(C(F)F)sc1C(=O)N[C@@H]1CCCN(c2cc(Br)n3ncnc(N)c23)C1]

Analogous to the procedure for the preparation of Example 428E, reaction of Example 428B (200 mg, 0.677 mmol) and (R)-5-(3-aminopiperidin-1-yl)-7-bromopyrrolo[2,1-f][1,2,4]triazin-4-amine (422 mg, 1.355 mmol) afforded Example 428C (475 mg, 62% yield) as a brown semisolid. MS: [M+H]⁺=588.2.

### Example 428

### Methyl (R)-3-(4-amino-5-(3-(5-(difluoromethyl)-3-(2-((methoxycarbonyl)amino)ethoxy)thiophene-2-carboxamido)piperidin-1-yl)pyrrolo[2,1-f][1,2,4]triazin-7-yl)-2,5-dihydro-1H-pyrrole-1-carboxylate

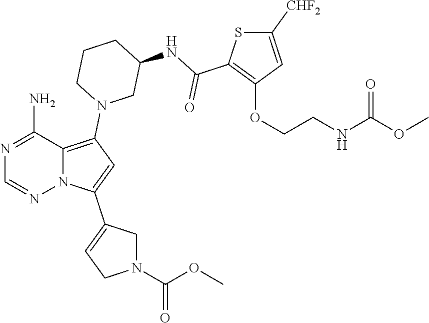 [SMILES: COC(=O)NCCOc1cc(C(F)F)sc1C(=O)N[C@@H]1CCCN(c2cc(C3=CCN(C(=O)OC)C3)n3ncnc(N)c23)C1]

Analogous to the procedure for the preparation of Example 276D, reaction of Example 428C (100 mg, 0.087 mmol) and methyl 3-(4,4,5,5-tetramethyl-1,3,2-dioxaborolan-2-yl)-2,5-dihydro-1H-pyrrole-1-carboxylate (21.94 mg, 0.087 mmol) afforded Example 428 (13.8 mg, 24% yield) as an off-white solid. LC-MS Method E: RT=1.651 min, [M+H]⁺=636.2; LC-MS Method F: RT=1.374 min, [M+H]⁺=635.3; ¹H NMR (400 MHz, DMSO-d₆) δ ppm 8.00 (br s, 1H), 7.86 (s, 1H), 7.54-7.40 (m, 3H), 7.24 (t, J=54.8 Hz, 1H), 6.78-6.63 (m, 2H), 4.53 (br d, J=11.0 Hz, 2H), 4.28-4.26 (m, 6H), 3.66 (d, J=4.8 Hz, 3H), 3.53 (s, 3H), 3.46-3.38 (m, 2H), 3.14 (br dd, J=1.5, 8.0 Hz, 1H), 3.05-2.94 (m, 1H), 2.77 (br s, 2H), 1.80 (br d, J=3.8 Hz, 3H), 1.62 (br s, 1H).

The following examples in Table 33 were prepared using the same procedure as shown in Example 428. Example 428C was coupled with the appropriate boronic acid or boronate ester.

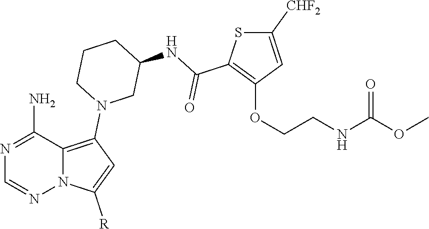

TABLE 33

| | | | | HPLC | |
|---|---|---|---|---|---|
| Ex- | | | LCMS | Method, | |
| ample | R | Name | (M + H)⁺ | RT (min.) | ¹H NMR |
| 429 | 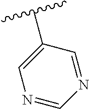 [SMILES: *c1cncnc1] | methyl (R)-(2-((2-((1-(4-amino- 7-(pyrimidin-5-yl)pyrrolo[2,1- f][1,2,4]triazin-5-yl)piperidin-3- yl)carbamoyl)-5- (difluoromethyl)thiophen-3- yl)oxy)ethyl)carbamate | 588.2 | E: 1.206 F: 1.453 | (400 MHz, DMSO-d₆) δ ppm 9.46 (s, 2H), 9.10 (s, 1H), 8.34 (s, 1H), 7.94 (s, 1H), 7.50 (s, 2H), 7.33-7.21 (m, 1H), 7.12-6.93 (m, 2H), 4.26 (br t, J = 5.3 Hz, 3H), 3.90 (s, 1H), 3.53 (s, 3H), 3.30-3.21 (m, 2H), 3.08 (brs, 1H), 2.79 (br s, 2H), 1.83 (br s, 4H), 1.65 (br s, 1H) |
| 431 | 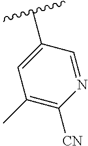 [SMILES: *c1cnc(C#N)c(C)c1] | methyl (R)-(2-((2-((1-(4-amino- 7-(6-cyano-5-methylpyridin-3- yl)pyrrolo[2,1-f] [1,2,4 ]triazin- 5-yl)piperidin-3-yl)carbamoyl)- 5-(difluoromethyl)thiophen-3- yl)oxy)ethyl)carbamate | 626.3 | E: 1.562 F: 1.837 | (400 MHz, DMSO-d₆) δ ppm 9.29 (d, J = 2.0 Hz,1H), 8.64 (d, J = 1.3 Hz, 1H), 8.27 (br s, 1H), 7.97 (s, 1H), 7.53-7.29 (m, 4H), 7.26-6.97 (m, 1H), 4.26 (brt, J = 4.5 Hz, 3H), 3.52 (s, 3H), 3.21 (m, 2H), 3.11-3.03 (m, 1H), 2.80 (br s, 2H), 2.54 (s, 3H), 1.97-1.78 (m, 4H), 1.63 (br s, 2H) |
| 432 | 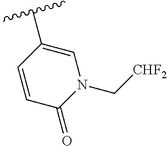 [SMILES: *c1ccc(=O)n(CC(F)F)c1] | (R)-3-(2-acetamidoethoxy)-N- (1-(4-amino-7-(1-(2,2-difluoro- ethyl)-6-oxo-1,6-dihydropyri- din-3-yl)pyrrolo[2,1-f] [1,2,4]triazin-5-yl)piperidin- 3-yl)-5-(difluoromethyl)thio- phene-2-carboxamide | 667.2 | E: 1.348 F: 1.561 | (400 MHz, DMSO-d₆) δ ppm 8.49 (d, J = 2.3 Hz, 1H), 8.10 (dd, J = 2.5, 9.5 Hz, 1H), 7.91 (s, 1H), 7.54-7.39 (m, 2H), 7.38-7.05 (m, 2H), 6.95 (s, 1H), 6.59 (d, J = 9.8 Hz, 1H), 6.52- 6.20 (m, 1H), 4.45 (dt, J = 4.0, 14.6 Hz, 2H), 4.25 (br t,J = 4.9 Hz, 3H), 3.53 (s, 3H), 3.4- 3.21 (m, 2H), 3.10-3.07 (m, 2H), 2.84-2.72 (m, 2H), 1.97-1.75 (m, 5H), 1.65 (br s, 1H) |

### Example 433

### Preparation of (R)—N-(1-(7-(1-acetyl-2, 5-dihydro-1H-pyrrol-3-yl)-4-aminopyrrolo [2,1-f][1,2,4]triazin-5-yl)piperidin-3-yl)-5-(difluoromethyl)-3-(oxetan-3-yloxy)thiophene-2-carboxamide

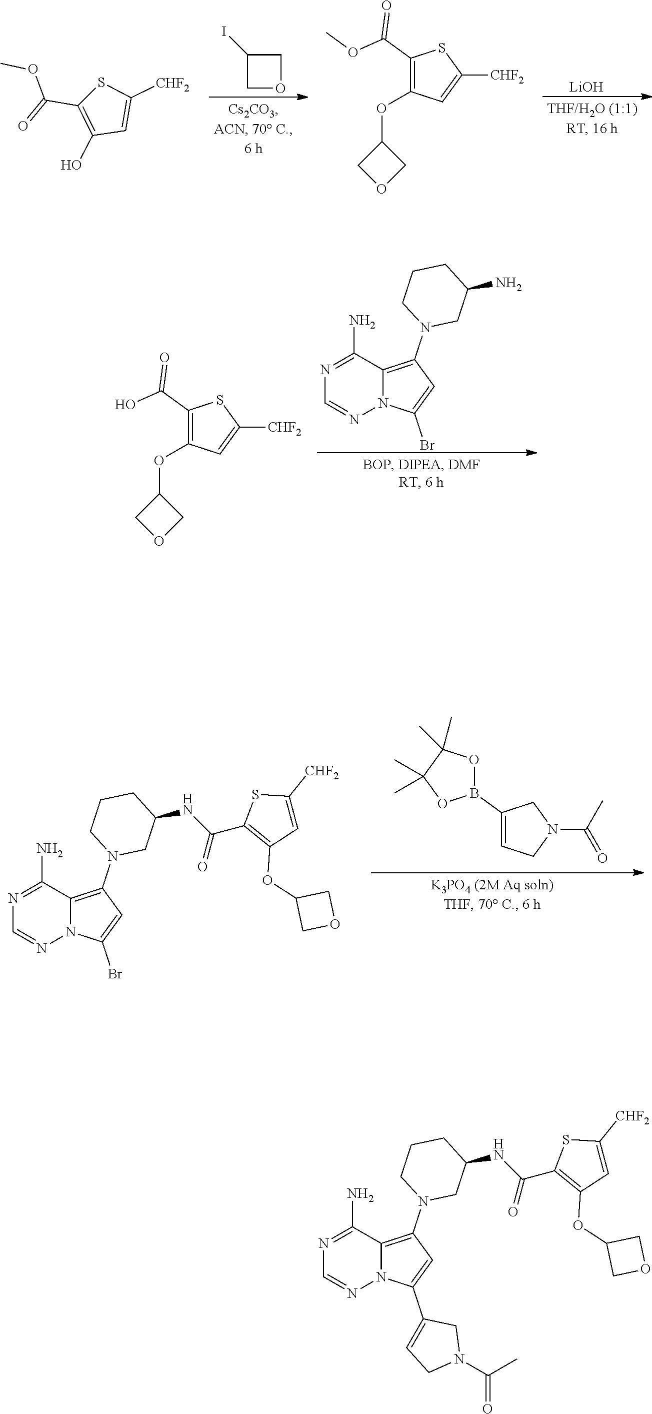

### Example 433A. Methyl 5-(difluoromethyl)-3-(oxetan-3-yloxy) thiophene-2-carboxylate

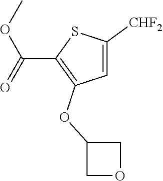 [SMILES: COC(=O)c1sc(C(F)F)cc1OC1COC1]

To a mixture of methyl 5-(difluoromethyl)-3-hydroxythiophene-2-carboxylate (1.5 g, 7.21 mmol) and cesium carbonate (7.04 g, 21.6 mmol) in acetonitrile (50 mL), was added 3-iodooxetane (3.17 mL, 36.0 mmol). The mixture was heated at 70° C. for 16 h followed by the addition of brine (80 mL) and ethyl acetate (50 mL). Organic layer was separated, dried over anhydrous sodium sulfate and concentrated to afford Example 433A (1.6 g, 69% yield) as an off-white solid. MS [M+H]⁺=265.2.

### Example 433B. 5-(difluoromethyl)-3-(oxetan-3-yloxy) thiophene-2-carboxylic acid

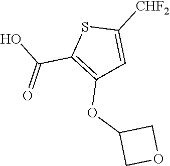 [SMILES: O=C(O)c1sc(C(F)F)cc1OC1COC1]

Analogous to the procedure for the preparation of Example 423D, reaction of Example 433A (1.3 g, 4.03 mmol) and LiOH (0.290 g, 12.1 mmol) afforded Example 433B (1.2 g) as an off-white solid. MS: [M−H]⁺=249.2.

### Example 433C. (R)—N-(1-(4-amino-7-bromopyrrolo [2,1-f][1,2,4]triazin-5-yl)piperidin-3-yl)-5-(difluoromethyl)-3-(oxetan-3-yloxy)thiophene-2-carboxamide

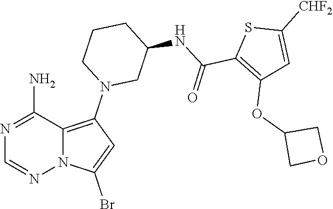 [SMILES: Nc1ncnn2c(Br)cc(N3CCC[C@@H](NC(=O)c4sc(C(F)F)cc4OC4COC4)C3)c12]

Analogous to the procedure for the preparation of Example 423E, reaction of (R)-5-(3-aminopiperidin-1-yl)-7-bromopyrrolo[2,1-f][1,2,4]triazin-4-amine (1.617 g, 5.20 mmol) and Example 433B (1.3 g, 5.20 mmol) afforded Example 433C (3.0 g, 60% yield) as a brown semisolid. MS: [M+H]⁺=543.2.

### Example 433

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-(oxetan-3-yloxy)thiophene-2-carboxamide

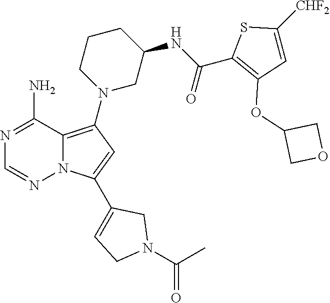 [SMILES: CC(=O)N1CC=C(c2cc(N3CCC[C@@H](NC(=O)c4sc(C(F)F)cc4OC4COC4)C3)c3c(N)ncnn23)C1]

Analogous to the procedure for the preparation of Example 276D, reaction of Example 433C (100 mg, 0.103 mmol) and 1-(3-(4,4,5,5-tetramethyl-1,3,2-dioxaborolan-2-yl)-2,5-dihydro-1H-pyrrol-1-yl)ethan-1-one (24.44 mg, 0.103 mmol) afforded Example 433 (6.8 mg, 12% yield) as an off-white solid. LC-MS Method E. RT=1.429 min, [M+H]⁺=574.2. LC-MS Method F: RT=1.176 min, [M+H]⁺=574.2; ¹H NMR (400 MHz, DMSO-d₆) δ ppm 8.01 (s, 1H), 7.87 (d, J=1.8 Hz, 1H), 7.68-7.49 (m, 1H), 7.37-7.06 (m, 2H), 6.81-6.69 (m, 2H), 5.49 (quin, J=5.3 Hz, 1H), 4.90 (q, J=6.7 Hz, 2H), 4.69 (br s, 1H), 4.65-4.55 (m, 2H), 4.53-4.42 (m, 2H), 4.31-4.14 (m, 2H), 3.18-3.08 (m, 1H), 3.00-2.80 (m, 3H), 2.03 (d, J=16.8 Hz, 3H), 1.92-1.75 (m, 3H), 1.66 (br s, 1H).

The following Examples in Table 34 were prepared using the same procedure as shown in Example 433. Example 433C was coupled with the appropriate boronic acid or boronate ester.

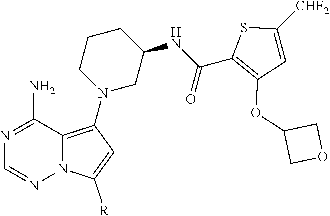

TABLE 34

| | | | | HPLC | |
|---|---|---|---|---|---|
| | | | LCMS | Method, | |
| | | | (M + | RT | |
| R | Structure | Name | H)⁺ | (min.) | ¹HNMR |
| 434 | 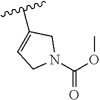 [SMILES: *C1=CCN(C(=O)OC)C1] | methyl (R)-3-(4-amino-5-(3-(5- (difluoromethyl)-3-(oxetan-3- yloxy)thiophene-2- carboxamido)piperidin-1- yl)pyrrolo[2,1-f][1,2,4]triazin- 7-yl)-2,5-dihydro-1H-pyrrole- 1-carboxylate | 590.2 | E: 1.372 F: 1.680 | (400 MHz, DMSO-d₆) δ ppm 7.86 (s, 2H), 7.61 (br s, 1H), 7.38-7.06 (m, 2H), 6.83-6.68 (m, 2H), 5.54-5.43 (m, 1H), 4.91 (q, J = 6.8 Hz, 2H), 4.66-4.48 (m, 4H), 4.35- 4.14 (m, 3H), 3.66 (d, J = 4.3 Hz, 3H), 3.17-3.10 (m, 1H), 2.87 (m, 3H), 1.94-1.78 (m, 3H), 1.73-1.59 (m, 1H) |
| 435 | 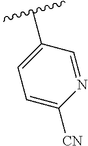 [SMILES: *c1ccc(C#N)nc1] | (R)-N-(1-(4-amino-7-(6- cyanopyridin-3-yl)pyrrolo[2,1- f][1,2,4]triazin-5-yl)piperidin- 3-yl)-5-(difluoromethyl)-3- (oxetan-3-yloxy)thiophene-2- carboxamide | 567.2 | E: 1.419 F: 1.720 | (400 MHz, DMSO-d₆) δ ppm 9.45 (s, 1H), 8.82 (dd, J = 2.1, 8.1 Hz, IH), 8.10 (d, J = 8.5 Hz, 1H), 7.96 (s, 1H), 7.63 (br s, IH), 7.45 (s, 1H), 7.23 (s, 2H), 5.54-5.44 (m, 1H), 4.97-4.86 (m, 2H), 4.67-4.58 (m, 2H), 4.25 (br s, 1H), 3.18 (s, 1H), 2.94 (br s, 3H), 1.88 (br s, 3H), 1.70 (br s, 1H) |
| 436 | 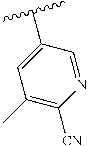 [SMILES: *c1cnc(C#N)c(C)c1] | (R)-N-(1-(4-amino-7-(6-cyano- 5-methylpyridin-3- yl)pyrrolo[2,1-f][1,2,4]triazin- 5-yl)piperidin-3-yl)-5- (difluoromethyl)-3-(oxetan-3- yloxy)thiophene-2- carboxamide | 582.2 | E: 1.795 F: 1.787 | (400 MHz, DMSO-d₆) δ ppm 9.30 (d, J = 1.5 Hz, 1H), 8.69-8.61 (m, 1H), 8.29-8.03 (m, 1H), 7.95 (s, IH), 7.61 (br s, 1H), 7.40 (s, 1H), 7.36-7.06 (m, 2H), 6.87 (s, 1H), 5.55-5.44 (m, 1H), 4.97-4.85 (m, 2H), 4.67-4.54 (m, 2H), 4.23 (br s, 1H), 3.17 (br d, J = 5.0 Hz, 1H), 3.07-2.79 (m, 3H), 2.53 (s, 3H), 1.96-1.79 (m, 3H), 1.67 (br s, 1H) |
| 437 | 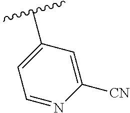 [SMILES: *c1ccnc(C#N)c1] | (R)-N-(1-(4-amino-7-(2- cyanopyridin-4-yl)pyrrolo[2,1- f][1,2,4]triazin-5-yl)piperidin- 3-yl)-5-(difluoromethyl)-3- (oxetan-3-yloxy)thiophene-2- carboxamide | 567.2 | E: 1.440 F: 1.727 | (400 MHz, DMSO-d₆) δ ppm 8.77-8.71 (m, 2H), 8.54- 8.48 (m, 1H), 8.32 (s. 1H), 8.02 (s, 1H), 7.57 (s, 2H), 7.37-7.07 (m, 2H), 5.53-5.46 (m, 1H), 4.93-4.87 (m, 2H), 4.66-4.57 (m, 2H), 4.24 (br s, 1H), 3.17 (s, 1H), 2.89 (br s, 3H), 1.86 (br s, 3H), 1.69 (br s, 1H) |

### Example 438

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-((1-methylpiperidin-4-yl)oxy)thiophene-2-carboxamide

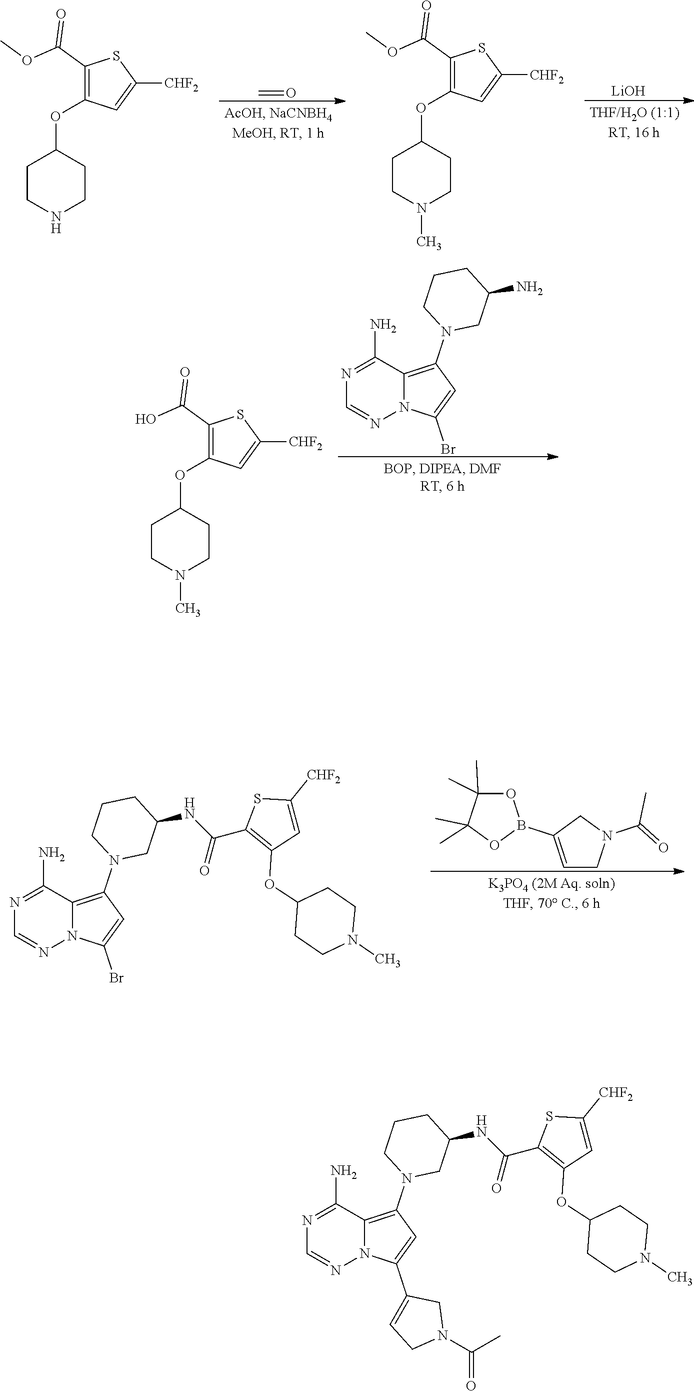

### Example 438A. 5-(difluoromethyl)-3-((1-methylpiperidin-4-yl) oxy) thiophene-2-carboxylic acid

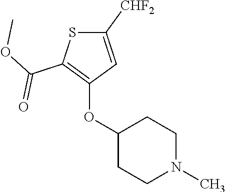 [SMILES: COC(=O)c1sc(C(F)F)cc1OC1CCN(C)CC1]

To an ice cold solution of methyl 5-(difluoromethyl)-3-(piperidin-4-yloxy)thiophene-2-carboxylate (1 g, 3.43 mmol) in methanol (2 mL), was added formaldehyde (36% in water, 0.263 mL, 3.43 mmol) and acetic acid (0.020 mL, 0.343 mmol). The mixture was stirred at room temperature for 20 min and then cooled to 0° C. Sodium cyanoborohydride (0.324 g, 5.15 mmol) was added and stirred at room temperature for 16 h. The reaction mixture was partitioned between ethyl acetate (50 mL) and ammonium chloride solution (50 mL). Organic layer was separated, dried over sodium sulphate and concentrated which afforded Example 438A (950 mg, 91% yield) as a brown semisolid. MS: [M+H]⁺=306.2.

### Example 438B. 5-(difluoromethyl)-3-((1-methylpiperidin-4-yl) oxy) thiophene-2-carboxylic acid

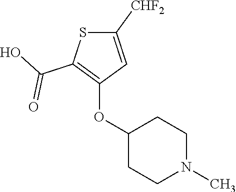 [SMILES: CN1CCC(Oc2cc(C(F)F)sc2C(=O)O)CC1]

Analogous to the procedure for the preparation of Example 423D, reaction of Example 438A (900 mg, 2.95 mmol) and LiOH (212 mg, 8.84 mmol) afforded Example 438B (800 mg, 82% yield) as an off-white solid. MS: [M+H]⁺=292.2.

### Example 438C. (R)—N-(1-(4-amino-7-bromopyrrolo [2,1-f][1,2,4]triazin-5-yl)piperidin-3-yl)-5-(difluoromethyl)-3-((1-methylpiperidin-4-yl)oxy)thiophene-2-carboxamide

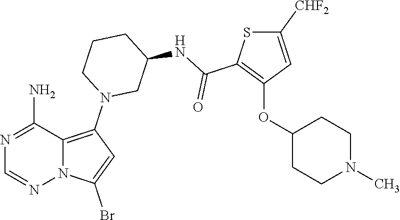 [SMILES: CN1CCC(Oc2cc(C(F)F)sc2C(=O)N[C@@H]2CCCN(c3cc(Br)n4ncnc(N)c34)C2)CC1]

Analogous to the procedure for the preparation of Example 423E, reaction of (R)-5-(3-aminopiperidin-1-yl)-7-bromopyrrolo[2,1-f][1,2,4]triazin-4-amine (855 mg, 2.75 mmol) and Example 438B (800 mg, 2.75 mmol) afforded Example 438C (1.8 g, 44% yield) as an off-white solid. MS: [M+H]⁺=584.3.

### Example 438

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(difluoromethyl)-3-((1-methylpiperidin-4-yl)oxy)thiophene-2-carboxamide

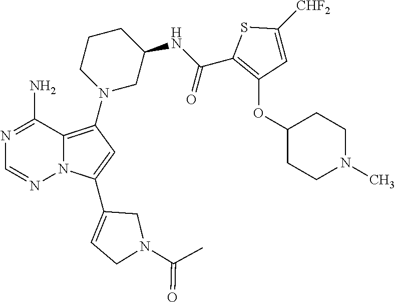 [SMILES: CC(=O)N1CC=C(c2cc(N3CCC[C@@H](NC(=O)c4sc(C(F)F)cc4OC4CCN(C)CC4)C3)c3c(N)ncnn23)C1]

Analogous to the procedure for the preparation of Example 276D, reaction of Example 438C (100 mg, 0.067 mmol) and 1-(3-(4,4,5,5-tetramethyl-1,3,2-dioxaborolan-2-yl)-2,5-dihydro-1H-pyrrol-1-yl)ethan-1-one (15.8 mg, 0.067 mmol) afforded Example 438 (10.9 mg, 25% yield) as an off-white solid. LC-MS Method E: RT=1.340 min, [M+H]⁺=615.2; LC-MS Method F: RT=0.960 min, [M+H]⁺=615.3; ¹H NMR (400 MHz, DMSO-d₆) δ ppm 7.86 (d, J=1.5 Hz, 1H), 7.58 (s, 2H), 7.23 (t, J=54.3 Hz, 1H), 6.82-6.65 (m, 2H), 4.74-4.58 (m, 2H), 4.53-4.41 (m, 2H), 4.33-4.09 (m, 2H), 3.17 (br d, J=5.0 Hz, 1H), 2.95 (br s, 1H), 2.80 (br s, 2H), 2.61-2.53 (m, 2H), 2.33 (td, J=1.8, 3.6 Hz, 2H), 2.18 (br d, J=1.5 Hz, 3H), 2.07-1.88 (m, 6H), 1.86-1.69 (m, 4H), 1.54 (br s, 1H).

The following examples in Table 35 were prepared using the same procedure as shown in Example 438. Example 438C was coupled with the appropriate boronic acid or boronate ester.

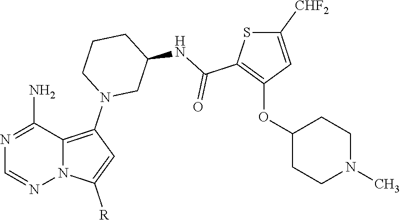

TABLE 35

| | | | | HPLC | |
|---|---|---|---|---|---|
| | | | LCMS | Method, | |
| Example | R | Name | (M + H)⁺ | RT (min.) | ¹H NMR |
| 439 | 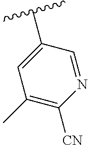 [SMILES: *c1cnc(C#N)c(C)c1] | (R)-N-(1-(4-amino-7-(6- cyano-5-methylpyridin-3- yl)pyrrolo[2,1- f][1,2,4]triazin-5- yl)piperidin-3-yl)-5- (difluoromethyl)-3-((1- methylpiperidin-4- yl)oxy)thiophene-2- carboxamide | 622.3 | E: 1.214 F: 1.715 | (400 MHz, DMSO-d₆) δ ppm 9.29 (d, J = 1.8 Hz, 1H), 8.65 (d, J = 1.5 Hz, 1H), 8.32-8.04 (m, 1H), 7.95 (s, 1H), 7.66-7.19 (m, 4H), 6.84 (s, 1H), 4.69-4.54 (m, 1H), 4.27-4.15 (m, 1H), 3.25- 3.19 (m, 1H), 3.05-2.99 (m, 1H), 2.88 (br s, 2H), 2.53 (s, 3H), 2.42-2.09 (m, 6H), 2.03-1.82 (m, 6H), 1.80-1.69 (m, 2H), 1.57 (br s, 1H) |
| 440 | 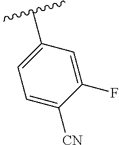 [SMILES: *c1ccc(C#N)c(F)c1] | (R)-N-(1-(4-amino-7-(4- cyano-3- fluorophenyl)pyrrolo[2,1- f][1,2,4]triazin-5- yl)piperidin-3-yl)-5- (difluoromethyl)-3-((1- methylpiperidin-4- yl)oxy)thiophenc-2- carboxamide | 625.3 | E: 1.320 F: 1.793 | (400 MHz, DMSO-d₆) δ ppm 9.63 (br s, 1H), 8.40-8.14 (m, 3H), 8.03-7.92 (m, 2H), 7.64-7.43 (m, 2H), 7.42-7.20 (m, 1H), 7.14- 6.94 (m, 1H), 4.86-4.65 (m, 1H), 4.28-4.13 (m, 1H), 3.39 (br dd, J = 2.4, 4.1 Hz, 3H), 3.14-2.98 (m, 4H), 2.93-2.70 (m, 4H), 2.41- 2.34 (m, 1H), 2.22-2.13 (m, 1H), 2.08-2.01 (m, 1H), 1.95-1.48 (m, 5H) |

### Example 441

### Preparation of Methyl (R)-3-(4-amino-5-(3-(5-(difluoromethyl)-3-((1-isopropylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

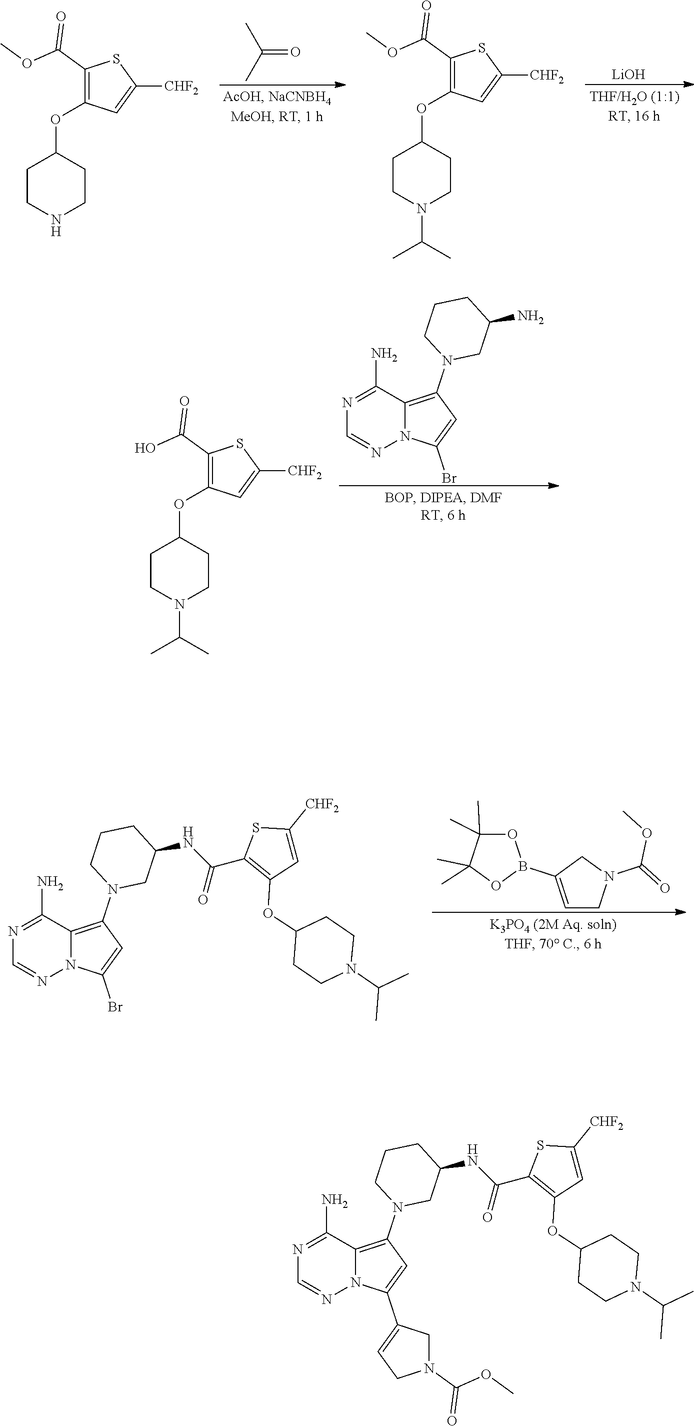

### Example 166A. Methyl 5-(difluoromethyl)-3-((1-isopropylpiperidin-4-yl)oxy)thiophene-2-carboxylate

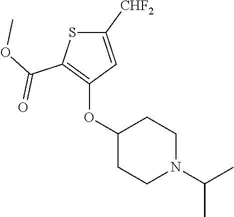 [SMILES: COC(=O)c1sc(C(F)F)cc1OC1CCN(C(C)C)CC1]

Analogous to the procedure for the preparation of Example 438A, reaction of methyl 5-(difluoromethyl)-3-(piperidin-4-yloxy)thiophene-2-carboxylate (250 mg, 0.858 mmol) and acetone (0.076 mL, 1.03 mmol) afforded Example 441A (360 mg, 76% yield) as an off-white semisolid. MS: [M+H]⁺=334.3.

### Example 441B. 5-(difluoromethyl)-3-((1-isopropylpiperidin-4-yl) oxy) thiophene-2-carboxylic acid

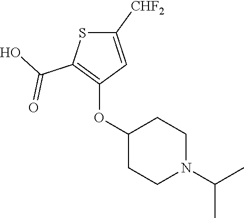 [SMILES: CC(C)N1CCC(Oc2cc(C(F)F)sc2C(=O)O)CC1]

Analogous to the procedure for the preparation of Example 423D, reaction of Example 441A (320 mg, 0.576 mmol) and LiOH (41.4 mg, 1.73 mmol) afforded Example 441B (190 mg, 75% yield) as an off-white solid. MS: [M+H]⁺=320.3.

### Example 441C. (R)—N-(1-(4-amino-7-bromopyrrolo [2,1-f][1,2,4]triazin-5-yl)piperidin-3-yl)-5-(difluoromethyl)-3-((1-isopropylpiperidin-4-yl)oxy)thiophene-2-carboxamide

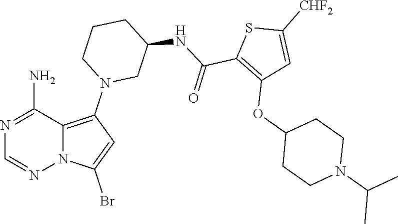 [SMILES: CC(C)N1CCC(Oc2cc(C(F)F)sc2C(=O)N[C@@H]2CCCN(c3cc(Br)n4ncnc(N)c34)C2)CC1]

Analogous to the procedure for the preparation of Example 423E, reaction of (R)-5-(3-aminopiperidin-1-yl)-7-bromopyrrolo[2,1-f][1,2,4]triazin-4-amine (341 mg, 1.10 mmol) and Example 441B (175 mg, 0.548 mmol) afforded Example 441C (800 mg, 62% yield) as a brown solid. MS: [M+H]⁺=612.3.

### Example 441

### Methyl (R)-3-(4-amino-5-(3-(5-(difluoromethyl)-3-((1-isopropylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

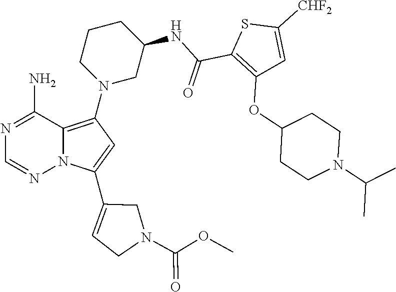 [SMILES: COC(=O)N1CC=C(c2cc(N3CCC[C@@H](NC(=O)c4sc(C(F)F)cc4OC4CCN(C(C)C)CC4)C3)c3c(N)ncnn23)C1]

Analogous to the procedure for the preparation of Example 276D, reaction of Example 441C (100 mg, 0.041 mmol) and methyl 3-(4,4,5,5-tetramethyl-1,3,2-dioxaborolan-2-yl)-2,5-dihydro-1H-pyrrole-1-carboxylate (10.3 mg, 0.041 mmol) afforded Example 441 (11.8 mg, 41% yield) as an off-white solid. LC-MS Method E: RT=1.636 min, [M+H]⁺=659.3; LC-MS Method F: RT=1.156 min, [M+H]⁺=659.3.

The following example in Table 36 was prepared using the same procedure as shown in Example 441. Example 441C was coupled with the p appropriate boronic acid or boronate ester.

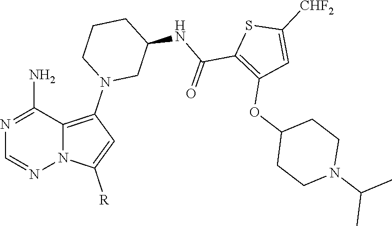

TABLE 36

| Ex- | | | LCMS | HPLC | |
|---|---|---|---|---|---|
| am- | | | (M + | Method, | |
| ple | R | Name | H)⁺ | RT (min.) | ¹H NMR |
| 442 | 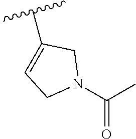 [SMILES: *C1=CCN(C(C)=O)C1] | (R)-N-(1-(7-(1-acetyl-2,5- dihydro-1H-pyrrol-3-yl)-4- aminopyrrolo[2,1- f][1,2,4]triazin-5- yl)piperidin-3-yl)-5- (difluoromethyl)-3-((1- isopropylpiperidin-4- yl)oxy)thiophone-2- carboxamide | 643.3 | E: 1.435 F: 1.032 | (400 MHz, DMSO-d₆) δ ppm 7.86 (d, J = 1.3 Hz, 1H), 7.57 (d, J = 1.3 Hz, 1H), 7.23 (t, J = 53.0 Hz, 1H), 6.77 (dd, J = 1.5, 10.3 Hz, 1H), 6.71 (d. J = 11.5 Hz, 1H), 4.68 (br d, J = 2.3 Hz, 1H), 4.53- 4.41 (m, 1H), 4.31-4.13 (m, 2H), 3.30-3.17 (m, 3H), 3.01-2.71 (m, 3H), 2.64-2.56 (m, 3H), 2.44-2.23 (m, 2H), 2.06-1.87 (m, 4H), 1.85-1.65 (m, 5H), 1.60-1.50 m, 2H), 0.93 (m, 6H). |

The following examples in Table 37 were prepared by treating Intermediate 32 with respective acids through acid-amine coupling.

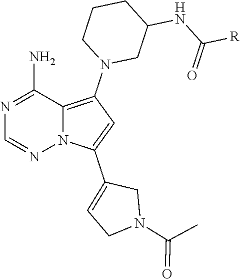

TABLE 37

| | | | | HPLC | |
|---|---|---|---|---|---|
| Ex- | | | LCMS | Method, | |
| am- | | | (M + | RT | |
| ple | R | Name | H)⁺ | (min.) | ¹H NMR |
| 443 | 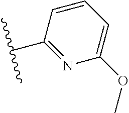 [SMILES: *c1cccc(OC)n1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-6- methoxypicolinamide | 477.3 | E: 1.570 F: 1.327 | (400 MHz, DMSO-d₆) δ ppm 8.29 (br d, J = 7.5 Hz, 1H), 8.08-7.72 (m, 3H), 7.62 (d, J = 7.3 Hz, 1H), 7.03 (d, J = 8.3 Hz, IH), 6.84-6.62 (m, 3H), 4.69 (br d, J = 1.5 Hz, 1H), 4.48 (br d, J = 8.5 Hz, 2H), 4.33- 4.15 (m, 2H), 3.97 (s, 3H), 3.19-3.15 (m, 1H), 3.03- 2.96 (m, 1H), 2.89-2.72 (m, 2H), 2.03 (d, J = 16.0 Hz, 3H), 1.84 (br s, 3H), 1.65 (br s, 1H) |
| 444 | 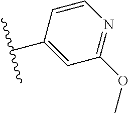 [SMILES: *c1ccnc(OC)c1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-2- methoxyisonicotinamide | 477.2 | E: 1.290 F: 1.033 | (400 MHz, DMSO-d₆) δ ppm 8.58 (br d, J = 7.8 Hz, 2H), 8.28 (d, J = 5.3 Hz, 1H), 7.97 (s, 1H), 7.33 (d, J = 5.5 Hz, 2H), 7.18 (s, 1H), 6.87-6.71 (m, 2H), 4.70 (br s, 1H), 4.50 (br d, J = 11.8 Hz, 2H), 4.28 (br s, 1H), 4.23-4.16 (m, 1H), 3.89 (s, 3H), 3.21 (br d, J = 10.8 Hz, 1H), 3.07- 3.00 (m, 1H), 2.75-2.67 (m, 2H), 2.03 (d, J = 15.5 |
| | | | | | Hz, 3H), 1.94-1.80 (m, 3H), 1.54 (brd, J = |
| | | | | | 5.5 Hz, 1H) |
| 445 | 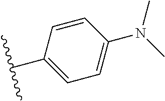 [SMILES: *c1ccc(N(C)C)cc1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-4- dimethylamino)benz- amide | 489.3 | E: 1.454 F: 1.131 | (400 MHz, DMSO-d₆) δ ppm 7.94 (br d, J = 8.0 Hz, 2H), 7.88 (d, J = 1.3 Hz, IH), 7.73 (d, J = 8.8 Hz, 2H), 6.95 (s, 1H), 6.81-6.75 (m, 1H), 6.74-6.63 (m, 3H), 4.69 (br s, 1H), 4.54-4.43 (m, 2H), 4.27 (br d, J = 2.3 Hz, 1H), 4.13 (br s, 1H), 3.18 (br s, 1H), 3.02 (br d, J = 1.8 Hz, 1H), 2.96 (s, 6H), 2.69- 2.62 (m, 2H), 2.03 (d, J = 15.8 Hz, 3H), 1.96-1.73 |
| | | | | | (m, 3H), 1.52 (br d, J = 9.5 Hz, 1H) |
| 446 | 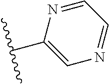 [SMILES: *c1cnccn1] | (R)-N-(1-(7-(1-acetyl- 2,5-dihydro-1H-pyrrol- 3-yl)-4-aminopyrrolo [2,1-][1,2,4]triazin-5- yl)piperidin-3- yl)pyrazine-2- | 448.3 | E: 1.206 F: 1.017 | (400 MHz, DMSO-d₆) δ ppm 9.18 (s, 1H), 8.94-8.86 (m, 1H), 8.83-8.68 (m, 2H), 7.89 (s, 1H), 6.82-6.67 (m, 2H), 4.69 (br s, 1H), 4.49 (brd, J = 10.0 Hz, 2H), 4.27 (br d, J = 1.0 Hz, 2H), 3.18-3.14 (m, 1H), 3.04-2.97 (m, 1H), 2.84-2.70 (m, 2H), 2.03 (d, J = 16.0 Hz, 3H), 1.86 (br d, J = 1.3 Hz, 3H), 1.72-1.61 |
| | | carboxamide | | | (m, 1H) |
| 447 | 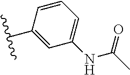 [SMILES: *c1cccc(NC(C)=O)c1] | (R)-3-acetamido-N-(1- (7-(1-acetyl-2,5-di- hydro-1H-pyrrol-3- yl)-4-aminopyrrolo [2,1-f][1,2,4]triazin- | 503.3 | E: 1.198 F: 1.044 | (400 MHz, DMSO-d₆) δ ppm 10.05 (s, 1H), 8.33 (br d, J = 7.5 Hz, 1H), 8.13-7.82 (m, 3H), 7.75 (br d, J = 8.0 Hz, 1H), 7.49 (d, J = 7.8 Hz, IH), 7.40-7.30 (m, 1H), 6.97 (s, 1H), 6.78 (br d, J = 10.5 Hz, 1H), 6.72 (d, J = 13.8 Hz, |
| | | 5-yl)piperidin-3- | | | 1H), 4.69 (br s, 1H), 4.48 (br d, J = 11.3 Hz, 2H), |
| | | yl)benzamide | | | 4.27 (br s, 1H), 4.21-4.13 (m, 1H), 3.21-3.16 (m, |
| | | | | | 1H), 3.00 (br d, J = 2.3 Hz, 1H), 2.68 (br s, 2H), |
| | | | | | 2.10-1.96 (m, 6H), 1.95-1.73 (m, 3H), 1.54 (br s, |
| | | | | | 1H) |
| 448 | 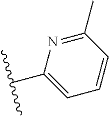 [SMILES: *c1cccc(C)n1] | (R)-N-(1-(7-(1-acetyl- 2,5-dihydro-1H-pyrrol- 3-yl)-4-aminopyrrolo [2,1-][1,2,4]triazin-5- yl)piperidin-3-yl)-6- methylpicolinamide | 461.3 | E: 1.447 F: 1.171 | (400 MHz, DMSO-d₆) δ ppm 8.49 (br d, J = 8.0 Hz, 1H), 8.14 (br s, 1H), 7.90 (s, 1H), 7.89-7.77 (m, 2H), 7.47 (d, J = 7.3 Hz, 1H), 6.97 (s, 1H), 6.84- 6.68 (m, 2H), 4.69 (br s, 1H), 4.49 (br d, J = 10.5 Hz, 2H), 4.27 (br d, J = 2.5 Hz, 1H), 4.21 (br dd, J = 3.0, 4.3 Hz, 1H), 3.19 (br d, J = 10.5 Hz, 1H), 3.00 (br d, J = 6.5 Hz, 1H), 2.86-2.70 (m, 2H), 2.57 |
| | | | | | (s, 3H), 2.03 (d, J = 15.8 Hz, 3H), 1.83 (br d, J = |
| | | | | | 1.8 Hz, 3H), 1.71-1.59 (m, 1H) |
| 449 | 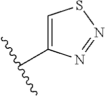 [SMILES: *c1csnn1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-1,2,3- thiadiazole-4- | 454.2 | E: 1.232 F: 1.041 | (400 MHz, DMSO-d₆) δ ppm 9.66 (s, 1H), 9.00 (dd, J = 5.0, 7.8 Hz, 1H), 8.08 (br s, 1H), 7.89 (s, 1H), 6.96 (s, 1H), 6.82-6.68 (m, 2H), 4.73 (s, IH), 4.49 (br d, J = 10.8 Hz, 2H), 4.27 (br s, 2H), 3.21 (br d, J = 11.0 Hz, 1H), 3.02 (br dd, J = 4.3, 10.3 Hz, 1H), 2.84-2.69 (m, 2H), 2.03 (d, J = 16.0 Hz, 3H), 1.97-1.76 (m, 4H), 1.72-1.59 (m, 1H) |
| 450 | 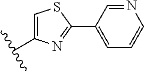 [SMILES: *c1csc(-c2cccnc2)n1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-2- | 530.2 | E: 1.363 F: 0.996 | (400 MHz, DMSO-d₆) δ ppm 9.31 (s, 1H), 8.78-8.69 (m, 1H), 8.44 (br t, J = 8.4 Hz, 2H), 8.39 (s, 1H), 7.97 (s, 1H), 7.60 (br dd, J = 4.9, 7.8 Hz, 1H), 7.25 (s, 1H), 6.87-6.74 (m, 1H), 4.70 (br d, J = 2.0 Hz, 1H), 4.49 (br d, J = 13.4 Hz, 2H), 4.27 (br s, 2H), |
| | | (pyridin-3-yl)thiazole-4- | | | 3.25-3.20 (m, 1H), 3.11-3.04 (m, 1H), 2.88-2.71 |
| | | carboxamide | | | (m, 2H), 2.07-1.80 (m, 4H), 1.70-1.61 (m, 1H) |
| 451 | 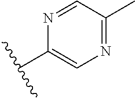 [SMILES: *c1cnc(C)cn1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-5- methylpyrazine-2- carboxamide | 462.3 | E: 1.341 F: 1.125 | (400 MHz, DMSO-d₆) δ ppm 9.04 (s, 1H), 8.69 (dd, J = 4.5, 8.0 Hz, 1H), 8.63 (s, 1H), 7.87 (d, J = 1.8 Hz, 1H), 6.77 (br d, J = 11.0 Hz, 1H),6.71 (d, J = 11.5 Hz, 1H), 4.69 (br s, 1H), 4.48 (br d, J = 9.3 Hz, 2H), 4.29-4.13 (m, 2H), 3.14 (br d, J = 10.3 Hz, 1H), 3.05-2.96 (m, 1H), 2.88-2.70 (m, 2H), 2.60 (s, 3H), 2.03 (d, |
| | | | | | J = 16.0 Hz, 3H), 1.85 (br s, 3H), 1.69 (br s, 1H) |
| 452 | 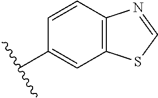 [SMILES: *c1ccc2ncsc2c1] | (R)-N-(1-(7-(1-acetyl- 2,5-dihydro-1H-pyrrol- 3-yl)-4-aminopyrrolo [2,1-f][1,2,4]triazin- 5-yl)piperidin-3- yl)benzo[d]thiazole-6- | 503.2 | E: 1.414 F: 1.189 | (400 MHz, DMSO-d₆) δ ppm 9.53 (s, 1H), 8.65 (s, 1H), 8.53 (br d, J = 7.8 Hz, IH), 8.38 (br s, 1H), 8.15 (dd, J = 1.0, 8.5 Hz, 1H), 8.01 (dd, J = 1.3, 10.0 Hz, 1H), 7.94 (s, 1H), 7.23 (s, 1H), 6.84-6.73 (m, 2H), 4.70 (br d, J = 1.8 Hz, 1H), 4.55-4.43 (m, 2H), 4.33-4.21 (m, 2H), 3.24 (br d, J = 5.0 Hz, 1H), |
| | | carboxamide | | | 3.04 (br dd, J = 3.9, 4.9 Hz, 1H), 2.72 (br d, J = |
| | | | | | 7.5 Hz, 2H), 2.03 (d, J = 15.3 Hz, 3H), 1.97- |
| | | | | | 1.77 (m, 3H), 1.64-1.52 (m, 1H) |
| 453 | 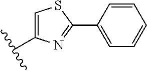 [SMILES: *c1csc(-c2ccccc2)n1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-2- | 529.2 | B: 1.790 F: 1.499 | (400 MHz, DMSO-d₆) δ ppm 8.36-8.23 (m, 2H), 8.13-8.02 (m, 2H), 7.88 (d, J = 1.5 Hz, 1H), 7.61-7.46 (m, 3H), 6.78 (br d, J = 10.3 Hz, 1H), 6.72 (d, J = 13.8 Hz, 1H), 4.69 (br s, 1H), 4.48 (br d, J = 12.0 Hz, 2H), 4.31- |
| | | phenylthiazole-4- | | | 4.16 (m, 2H), 3.19 (br dd, J = 4.9, 10.1 Hz, 1H), |
| | | carboxamide | | | 3.07-3.00 (m, 1H), 2.85-2.77 (m, 1H), 2.74-2.68 (m, |
| | | | | | 1H), 2.03 (d, J = 16.0 Hz, 3H), 1.98-1,90 (m, 1H), |
| | | | | | 1.85 (br d, J = 2.3 Hz, 2H), 1.65 (br s, IH) |
| 454 | 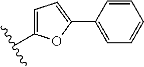 [SMILES: *c1ccc(-c2ccccc2)o1] | (R)-N-(1-(7-(1-acetyl- 2,5-dihydro-1H-pyrrol- 3-yl)-4-aminopyrrolo [2,1-f][1,2,4]triazin-5- yl)pipridin-3-yl)-5- | 512.2 | E: 1.722 F: 1.482 | (400 MHz, DMSO-d₆) δ ppm 8.31 (d, J = 8.3 Hz, 1H), 7.92 (d, J = 7.5 Hz, 3H), 7.87 (d, J = 1.3 Hz, 1H), 7.53-7.44 (m, 2H), 7.43-7.31 (m, 1H), 7.20 (dd, J = 1.6, 3.4 Hz, 1H), 7.09 (d. J = 3.5 Hz, 1H), 6.89 (br s, 1H), 6.78 (br d, J = 9.0 Hz, 1H), 6.72 (d, |
| | | phenylfuran-2- | | | J = 14.5 Hz, 1H), 4.69 |
| | | carboxamide | | | (br d, J = 1.0 Hz, 1H), 4.48 (br d, J = 11.3 Hz, 2H), |
| | | | | | 4.27 (brs, 1H), 4.23-4.11 (m, 1H), 3.20 (br dd, J = |
| | | | | | 5.4, 10.6 Hz, 1H), 3.10-3.03 (m, IH), 2.76-2.64 (m, |
| | | | | | 2H), 2.03 (d, J = 16.0 Hz, 3H), 1.97-1.90 (m, |
| | | | | | 1H), 1.85 (br s, 2H), 1.64-1.49 (m, 1H) |
| 455 | 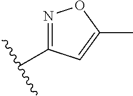 [SMILES: *c1cc(C)on1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-5- methylisoxazole-3- | Issue with ioniza- tion | E: 1.422 F: 1.190 | (400 MHz, DMSO-d₆) δ ppm 8.69 (dd, J = 2.6, 7.9 Hz, 1H), 8.40 (br s, IH), 7.95 (s, 1H), 7.22 (s, 1H), 6.84-6.66 (m, 2H), 6.52 (s, 1H), 4.70 (br s, 1H), 4.56-4.42 (m, 2H), 4.28 (br s, 1H), 4.23-4.15 (m, 1H), 3.18-3.13 (m, 1H), 3.03- 2.96 (m, 1H), 2.75-2.67 (m, 2H), 2.46 (s, 3H), 2.03 |
| | | | | | (d, J = 16.0 Hz, 3H), 1.85 (br s, 3H), 1.56 (br s, 1H) |
| 456 | 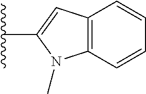 [SMILES: *c1cc2ccccc2n1C] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-1- methyl-1H-indole-2- | 499.3 | E: 1.696 F: 1.453 | (400 MHz, DMSO-d₆) δ ppm 8.43 (br d, J = 7.3 Hz, 1H), 7.99 (br s, 1H), 7.88 (d, J = 1.8 Hz, 1H), 7.64 (d, J = 8.0 Hz, 1H), 7.51 (d, J = 8.5 Hz, 1H), 7.27 (t, J = 7.6 Hz, 1H), 7.16-7.06 (m, 2H), 6.95 (br s, 1H), 6.81-6.67 (m, 2H), 4.70 (br s, 1H), 4.49 (br d, J = 12.3 Hz, 2H), 4.27 (br s, 1H), |
| | | carboxamide | | | 4.23-4.11 (m, 1H), 3.96 (s, 3H), |
| | | | | | 3.21 (br dd, J = 3.8, 5.0 Hz, 1H), 3.06-2.94 (m, |
| | | | | | 1H), 2.80-2.68 (m, 2H), 2.03 (d, J = 16.5 Hz, 3H), |
| | | | | | 1.91 (br s, 3H), 1.64-1.47 (m, 1H) |
| 457 | 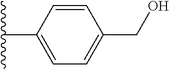 [SMILES: *c1ccc(CO)cc1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-4- | 476.2 | B: 1.163 F: 1.023 | (400 MHz, DMSO-d₆) δ ppm 8.46 (br s, 1H), 8.29 (d, J = 7.5 Hz, 1H), 7.95 (s, 1H), 7.81 (d, J = 8.3 Hz, 2H), 7.39 (d, J = 8.0 Hz, 2H), 7.23 (br s, 1H), 6.86-6.72 (m, 2H), 4.70 (br s, 1H), 4.55 (s, 2H), 4.50 (br d, J = 11.3 Hz, 2H), 4.28 (br s, 1H), 4.21 |
| | | (hydroxymethyl)benz- | | | (br s, 1H), 3.24-3.21 (m, 1H), 3.08 -3.02 (m, 1H), |
| | | amide | | | 2.74-2.68 (m, 2H), 2.03 (d, J = 15.3 Hz, 3H), |
| | | | | | 1.95-1.79 (m, 3H), 1.55 (br s, 1H) |
| 458 | 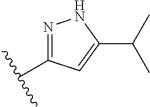 [SMILES: *c1cc(C(C)C)[nH]n1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-5- isopropyl-1H-pyrazole- 3-carboxamide | 478.3 | E: 1.408 F: 1.209 | (400 MHz, DMSO-d₆) δ ppm 12.89 (br s, 1H), 8.06-7.64 (m, 3H), 6.97-6.55 (m, 3H), 6.39 (s, 1H), 4.69 (br d, J = 2.0 Hz, 1H), 4.48 (br d, J = 8.5 Hz, 2H), 4.27 (br s, 1H), 4.13 (br s, 1H), 3.17-3.09 (m, 1H), 3.02-2.90 (m, 2H), 2.73- 2.66 (m, 2H), 2.03 (d, J = 16.0 Hz, 3H), 1.91-1.70 (m, 3H), 1.54 (br s, 1H), 1.22 (d, J = 6.8 Hz, 7H) |
| 459 | 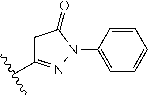 [SMILES: *C1=NN(c2ccccc2)C(=O)C1] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-5- oxo-1-phenyl-4,5- | 528.2 | E: 1.331 F: 1.373 | (400 MHz, DMSO-d₆) δ ppm 11.96 (br s, 1H), 7.94 (br dd, J = 3.4, 8.1 Hz, 2H), 7.88 (d, J = 1.8 Hz, 1H), 7.79 (d, J = 7.8 Hz, 2H), 7.50 (t, J = 7.9 Hz, 2H), 7.38-7.30 (m, 1H), 6.80-6.74 (m, 1H), 6.71 (d, J = 15.0 Hz, 1H), 5.89 (s, 1H), 4.69 (br d, J = 1.8 Hz, 1H), |
| | | dihydro-1H-pyrazole-3- | | | 4.48 (br d, J = 10.5 Hz, 2H), 4.27 (br d, J = |
| | | carboxamide | | | 1.5 Hz, 1H), 4.16 (br s, 1H), 3.16 (br dd, |
| | | | | | J = 4.5, 10.0 Hz, 1H), 3.02 (br d, J = |
| | | | | | 6.8 Hz, 1H), 2.76-2.64 (m, 2H), 2.03 (d, J = |
| | | | | | 15.8 Hz, 3H), 1.82 (br s, 3H), 1.58 (br s, 1H) |
| 460 | 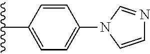 [SMILES: *c1ccc(-n2ccnc2)cc1] | (R)-N-(1-(7-(1-acetyl- 2,5-dihydro-1H-pyrrol- 3-yl)-4-aminopyrrolo [2,1-f][1,2,4]triazin-5- | 512.3 | E: 1.342 F: 1.026 | (400 MHz, DMSO-d₆) δ ppm 8.43 (br d, J = 7.5 Hz, 1H), 8.39 (s, 1H), 7.99 (d, J = 8.5 Hz, 2H), 7.89- 7.84 (m, 2H), 7.78 (dd, J = 1.0, 8.8 Hz, 2H), 7.15 (s, 1H), 6.91 (br s, 1H), 6.81-6.75 (m, 1H), 6.72 (d, |
| | | yl)piperidin-3-yl)-4- | | | J =13.3 Hz, 1H), 4.70 (br d, J = 1.3 Hz, 1H), 4.48 |
| | | (1H-imidazol-1-yl) | | | (br d, J = 11.0 Hz, 2H), 4.27 (br d, J = 2.8 Hz, 1H), |
| | | benzamide | | | 4.19 (dt, J = 1.9, 3.1 Hz, 1H), 3.23-3.17 (m, 1H), |
| | | | | | 3.08-2.97 (m, 1H), 2.77-2.66 (m, 2H), 2.03 (d, |
| | | | | | J = 16.3 Hz, 3H), 1.98-1.78 (m, 3H), 1.64-1.49 |
| | | | | | (m, 1H), 1.15 (br s, 1H) |
| 461 | 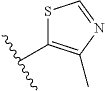 [SMILES: *c1scnc1C] | (R)-N-(1-(7-(1-acetyl- 2,5-dihydro-1H-pyrrol- 3-yl)-4-aminopyrrolo [2,1-f][1,2,4]triazin-5- yl)piperidin-3-yl)-4- methylthiazole-5- | 467.2 | E: 1.298 F: 1.097 | (400 MHz, DMSO-d₆) δ ppm 9.04 (d, J = 1.0 Hz, 1H), 8.27 (br d, J = 7.8 Hz, 1H), 7.97 (br s, 1H), 7.88 (d, J = 1.8 Hz, 1H), 6.93 (br s, 1H), 6.78 (br d, J = 10.3 Hz, 1H), 6.72 (d, J = 13.3 Hz, 1H), 4.70 (br s, 1H), 4.49 (br d, J = 10.0 Hz, 2H), 4.27 (br d, J = 2.0 Hz, 1H), |
| | | carboxamide | | | 4.14 (br s, 1H),3.20-3.16 (m, 1H), 2.96 (br s, |
| | | | | | 1H), 2.77-2.67 (m, 2H), 2.55 (s, 3H), 2.03 (d, J = |
| | | | | | 16.8 Hz, 3H), 1.95-1.68 (m, 3H), 1.52 (br s, 1H) |
| 462 | 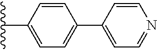 [SMILES: *c1ccc(-c2ccncc2)cc1] | (R)-N-(1-(7-(1-acetyl- 2,5-dihydro-1H-pyrrol- 3-yl)-4-aminopyrrolo [2,1-f][1,2,4]triazin- | 523.3 | E: 1.453 F: 1.055 | (400 MHz, DMSO-d₆) δ ppm 8.67 (d, J = 6.0 Hz, 2H), 8.48 (br d, J = 7.8 Hz, 1H), 8.09-7.84 (m, 6H), 7.80-7.71 (m, 2H), 6.93 (br s, 1H),6.81-6.75 (m, 1H), 6.72 (d, J = 12.0 Hz, 1H), 4.69 (br d, J = 1.5 |
| | | 5-yl)piperidin-3- | | | Hz, 1H), 4.48 (br d, J = 11.0 Hz, 2H), 4.27 (br d, |
| | | yl)-4-(pyridin-4- | | | J = 2.5 Hz, 1H), 4.20 (br dd, J = 2.3. 4.5 Hz, |
| | | yl)benzamide | | | 1H), 3.24-3.19 (m, 1H), 3.05-2.96 (m, 1H), |
| | | | | | 2.76-2.66 (m, 2H), 2.03 (d, J = 16.8 Hz, 3H), |
| | | | | | 1.98-1.77 (m, 3H), 1.56 (br d, J = 7.3 Hz, |
| 463 | 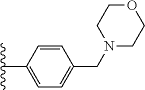 [SMILES: *c1ccc(CN2CCOCC2)cc1] | (R)-N-(1-(7-(1-acetyl- 2,5-dihydro-1H-pyrrol- 3-yl)-4-aminopyrrolo [2,1-f][1,2,4]triazin- 5-yl)piperidin-3- yl)-4-(morpholino- | 545.2 | E: 1.408 | (400 MHz, DMSO-d₆) δ ppm 8.31 (br d, J = 7.5 Hz, 1H), 7.87 (d, J = 1.8 Hz, 2H), 7.79 (d, J = 8.3 Hz, 2H), 7.38 (d, J = 7.3 Hz, 2H), 6.93-6.93 (m, 1H), 7.08-6.83 (m, 1H), 6.77 (td, J = 1.8, 10.3 Hz, 1H), 6.71 (d, J = 12.0 Hz, 1H), 4.69 (br d, J = 1.8 Hz, 1H), 4.48 (br d, J = 9.8 Hz, |
| | | methyl)benza- | | | 2H), 4.26 (br d, J = 2.8 Hz, 1H), 4.16 (br s, |
| | | mide | | | 1H), 3.60-3.55 (m, 4H), 3.51 (s, 2H), 3.21- |
| | | | | | 3.14 (m, 1H), 3.04-2.94 (m, 1H), |
| | | | | | 2.68 (td, J = 1.8, 3.7 Hz, 2H), 2.34 (br d, |
| | | | | | J = 4.3 Hz, 4H), 2.03 (d, J = 16.3 Hz, 3H), |
| | | | | | 1.90 (br d, J = 10.0 Hz, 3H), 1.57-1.46 (m, 1H) |
| 464 | 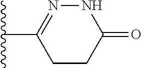 [SMILES: *C1=NNC(=O)CC1] | (R)-N-(1-(7-(1-acetyl- 2,5-dihydro-1H-pyrrol- 3-yl)-4-aminopyrrolo [2,1-f][1,2,4]triazin- 5-yl)piperidin- | 466.3 | E: 1.208 F: 1.052 | (400 MHz, DMSO-d₆) δ ppm 11.05 (s, 1H), 8.39 (br s, 1H), 7.98 (dd, J = 2.6, 7.9 Hz, 1H), 7.94 (s, 1H), 6.83-6.71 (m, 2H), 4.70 (br s, IH), 4.49 (br d, J = 10.0 Hz, 2H), 4.28 (br s, 1H), 4.12-4.03 (m, 1H), 3.06 (br s, 1H), 2.98 (br d, J = 7.8 Hz, 1H), 2.77- |
| | | 3-yl)-6-oxo-1,4,5,6- | | | 2.69 (m, 4H), 2.42-2.36 (m, 2H), 2.03 (d, J = 16.5 |
| | | tetrahydropyridazine- | | | Hz, 3H), 1.80 (br s, 3H), 1.64-1.52 (m, 1H) |
| | | 3-carboxamide | | | |
| 465 | 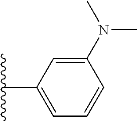 [SMILES: *c1cccc(N(C)C)c1] | (R)-N-(1-(7-(1-acetyl- 2,5-dihydro-1H-pyrrol- 3-yl)-4-aminopyrrolo [2,1-f][1,2,4]triazin- 5-yl)piperidin- 3-yl)-3-(dimethyl- amino)benza- mide | 489.3 | B: 1.477 F: 0.975 | (400 MHz, DMSO-d₆) δ ppm 8.21 (br d, J = 7.5 Hz, 1H), 7.97 (br s, 1H), 7.87 (d, J = 1.5 Hz, 1H), 7.30-7.16 (m, 1H), 7.14-7.05 (m, 2H), 6.98-6.81 (m, 2H), 6.78 (br d, J = 9.8 Hz, 1H), 6.71 (d, J = 14.0 Hz, 1H), 4.69 (br s, 1H), 4.48 (br d, J = 10.8 Hz, 2H), 4.27 (br s, 1H), 4.21-4.03 (m, 1H), 3.22-3.15 (m, 1H), 3.01 (br s, 1H), 2.93 (s, 6H), 2.71-2.64 (m, |
| | | | | | 2H), 2.03 (d, J = 16.0 Hz, 3H), 1.93-1.75 |
| | | | | | (m, 3H), 1.59-1.45 (m, 1H) |
| 466 | 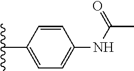 [SMILES: *c1ccc(NC(C)=O)cc1] | (R)-4-acetamido-N-(1- (7-(1-acetyl-2,5-di- hydro-1H-pyrrol-3- yl)-4-aminopyrrolo[2,1- f][1,2,4]triazin-5- | 503.3 | E: 1.187 F: 1.010 | (400 MHz, DMSO-d₆) δ ppm 10.13 (s, 1H), 8.25-8.17 (m, 1H), 8.10 (s, 1H), 7.89 (s, 1H), 7.79 (d, J = 8.5 Hz, 2H), 7.64 (d, J = 8.8 Hz, 2H), 6.96 (s, IH), 6.82-6.68 (m, 2H), 4.74-4.64 (m, 1H), 4.53-4.43 (m, 2H), |
| | | yl)piperidin-3- | | | 4.30-4.24 (m, 1H), 4.16 (dt, J = 1.9, 2.8 |
| | | yl)benzamide | | | Hz, 1H), 3.21-3.18 (m, 1H), 3.04-2.99 (m, |
| | | | | | 1H), 2.72-2.64 (m, 2H), 2.11-2.00 (m, 6H), |
| | | | | | 1.95-1.77 (m, 3H), 1.58-1.46 (m, 1H) |
| 467 | 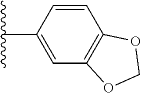 [SMILES: *c1ccc2c(c1)OCO2] | (R)-N-(1-(7-(1-acetyl- 2,5-dihydro-1H-pyrrol- 3-yl)-4-aminopyrrolo [2,1-f][1,2,4]triazin- 5-yl)piperidin-3- yl)benzo[d][1,3] | 490.3 | E: 1.362 F: 1.130 | (400 MHz, DMSO-d₆) δ ppm 8.17 (br d, J = 7.5 Hz, 1H), 7.96 (br s, 1H), 7.87 (s, 1H), 7.45 (d, J = 8.3 Hz, 1H), 7.39 (s, 1H), 6.98 (d, J = 8.0 Hz, 1H), 6.89 (br s, 1H), 6.77 (br d, J = 10.0 Hz, 1H), 6.71 (d, J = 13.8 Hz, 1H), 6.09 (s, 2H), 4.69 (br d, J = 1.3 Hz, 1H), 4.48 (br d, J = |
| | | dioxole-5- | | | 9.5 Hz, 2H), 4.26 (br s, 1H), 4.13 (br s, 1H), |
| | | carboxamide | | | 3.17 (br s, 1H), 3.01 (br s, 1H), |
| | | | | | 2.71-2.63 (m, 2H), 2.03 (d, J = 16.0 Hz, |
| | | | | | 3H), 1.95-1.70 (m, 3H), 1.58-1.45 (m, 1H) |
| 468 | 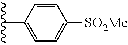 [SMILES: *c1ccc(S(C)(=O)=O)cc1] | f][1,2,4]triazin-5- (R)-N-(1-(7-(1-acetyl- 2,5-dihydro-1H-pyrrol- 3-yl)-4-aminopyrrolo | 524.3 | E: 1.217 F: 1.055 | (400 MHz, DMSO-d₆) δ ppm 8.64 (br d, J = 7.8 Hz, 1H), 8.44 (br s, 1H), 8.10-8.00 (m, 4H), 7.95 (s, 1H), 7.23 (s, 1H), 6.85-6.69 (m, 2H), 4.70 (br s, 1H), 4.50 (br d, J = 11.3 Hz, 2H), 4.32-4.16 (m, |
| | | [2,1-yl)piperidin- | | | 2H), 3.28-3.22 (m, 5H), 3.03 (br dd, J = 1.0, 9.5 |
| | | 3-yl)-4-(methyl- | | | Hz, 1H), 2.78-2.70 (m, 2H), 2.03 (d, J = 16.0 |
| | | sulfonyl)benzamide | | | Hz, 3H), 1.98-1.77 (m, 3H), 1.62-1.50 (m, 1H) |
| 470 | 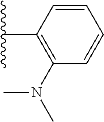 [SMILES: *c1ccccc1N(C)C] | (R)-N-(1-(7-(1-acetyl- 2,5-dihydro-1H-pyrrol- 3-yl)-4-aminopyrrolo [2,1-f][1,2,4]triazin- 5-yl)piperidin- 3-yl)-2-(dimethyl- amino)benzamide | 489.3 | E: 1.541 F: 0.928 | (400 MHz, DMSO-d₆) δ ppm 9.17 (br d, J = 6.3 Hz, 1H), 8.43 (br s, 1H), 7.95 (s, 1H), 7.67 (br d, J = 7.8 Hz, 1H), 7.53-7.43 (m, 1H), 7.31 (br dd, J = 1.5, 6.0 Hz, 1H), 7.25-7.07 (m, 2H), 6.85-6.74 (m, 2H), 4.71 (br s, 1H), 4.50 (br d, J = 11.0 Hz, 2H), 4.30-4.20 (m, 2H), 3.23 (br d, J = 2.0 Hz, 1H), 2.96 (br d, J = 3.8 Hz, 1H), 2.80 (br d, J = 2.3 Hz, 8H), 2.04 (d, J = 16.0 Hz, |
| | | | | | 3H), 1.96-1.77 (m, 3H), 1.54 (br s, 1H) |
| 471 | 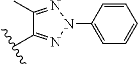 [SMILES: *c1nn(-c2ccccc2)nc1C] | (R)-N-(1-(7-(1-acetyl- 2,5-dihydro-1H-pyrrol- 3-yl)-4-aminopyrro- lo[2,1-f][1,2,4]tri- azin-5-yl)piperidin- | 527.3 | E: 1.795 F: 1.524 | (400 MHz, DMSO-d₆) δ ppm 8.43 (br dd, J = 3.4, 8.1 Hz, 2H), 8.11-8.02 (m, 2H), 7.94 (s, 1H), 7.60 (t, J = 7.9 Hz, 2H), 7.50-7.40 (m, 1H), 6.84-6.74 (m, 2H), 4.70 (br d, J = 2.0 Hz, 1H), 4.50 (br d, J = 13.3 Hz, 2H), 4.30-4.16 (m, 2H), 3.21 (brd, J = 11.3 Hz, |
| | | 3-yl)-5-methyl-2- | | | 1H), 3.06-3.01 (m, 1H), 2.82-2.70 (m, 2H), 2.03 (d, |
| | | phenyl-2H-1,2,3-tri- | | | J = 16.3 Hz, 3H), 1.97-1.81 (m, 3H), 1.64 (br s, 1H) |
| | | cazole-4-arboxamide | | | |
| 472 | 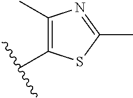 [SMILES: *c1sc(C)nc1C] | (R)-N-(1-(7-(1-acetyl- 2,5-dihydro-1H-pyrrol- 3-yl)-4-aminopyrrolo [2,1-f][1,2,4]triazin- 5-yl)piperidin-3- yl)-2,4-dimethylthia- | 481.2 | E: 1.242 F: 0.990 | (400 MHz, DMSO-d₆) δ ppm 8.52 (br s, 1H), 8.10 (br d, J = 7.8 Hz, IH), 7.96 (s, 1H), 7.30 (br s, 1H), 6.86-6.72 (m, 2H), 4.71 (br s, 1H), 4.50 (br d, J = 11.3 Hz, 2H), 4.28 (br d, J = 1.5 Hz, 1H), 4.14 (br dd, J = 3.1, 4.4 Hz, 1H), 3.19 (br d, J = 11.0 Hz, 1H), 3.00-2.93 (m, 1H), 2.77-2.67 (m, |
| | | zole-5-carboxamide | | | 2H), 2.61 (s, 3H), 2.47 (s, 3H), 2.04 (d, J = |
| | | | | | 16.3 Hz, 3H), 1.85 (br s, 3H), 1.53 (br s, 1H) |
| 473 | 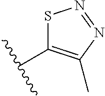 [SMILES: *c1snnc1C] | (R)-N-(1-(7-(1-acetyl- 2,5-dihydro-1H-pyrrol- 3-yl)-4-aminopyrrolo [2,1-f][1,2,4]triazin- 5-yl)piperidin-3- yl)-4-methyl-1,2,3-thia- | 468.2 | E: 1.247 F: 0.994 | (400 MHz, DMSO-d₆) δ ppm 8.86 (br d, J = 7.5 Hz, 1H), 8.43 (br s, 1H), 7.96 (s, IH), 7.22 (br s, 1H), 6.86-6.71 (m, 2H), 4.71 (br d, J = 1.3 Hz, 1H), 4.50 (br d, J = 10.5 Hz, 2H), 4.32-4.14 (m, 2H), 3.21 (br d, J = 8.5 Hz, 1H), 3.01-2.93 (m, 1H), 2.82-2.69 (m, 5H), 2.04 (d, J = 16.5 Hz, 3H), |
| | | diazole-5-carboxamide | | | 1.90 (br s, 3H), 1.53 (br s, 1H) |
| 474 | 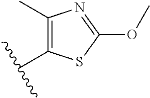 [SMILES: *c1sc(OC)nc1C] | (R)-N-(1-(7-(1-acetyl- 2,5-dihydro-1H-pyrrol- 3-yl)-4-aminopyrrolo [2,1-f][1,2,4]triazin- 5-yl)piperidin- 3-yl)-2-methoxy-4- | 497.2 | E: 1.322 F: 1.096 | (400 MHz, DMSO-d₆) δ ppm 8.50 (br s, 1H), 7.96 (s, 1H), 7.92 (br d, J = 7.5 Hz, 1H), 7.23 (s, 1H), 6.89-6.72 (m, 2H), 4.71 (br d, J = 1.8 Hz, 1H), 4.55- 4.45 (m, 2H), 4.28 (br s, 1H), 4.17-4.05 (m, 1H), 4.02 (s, 3H), 3.18 (br d, J = 10.5 Hz, 1H), 2.98 (br d, J = 8.5 Hz, 1H), 2.75-2.65 (m, 2H), 2.41 (s, |
| | | methylthiazole-5- | | | 3H), 2.03 (d, J = 16.3 Hz, 3H), 1.84 (br s, 3H), |
| | | carboxamide | | | 1.53 (br s, 1H) |
| 478 | 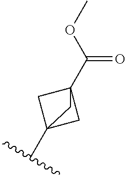 [SMILES: *C12CC(C(=O)OC)(C1)C2] | Methyl(R)-3-((1-(7-(1- acetyl-2,5-dihydro-1H- pyrrol-3-yl)-4- aminopyrrolo[2,1- f][1,2,4]triazin-5- yl)piperidin-3-yl) carbamoyl)bicyclo [1.1.1]pentane-1- carboxylate | 494.2 | E: 1.149 F: 0.925 | (400 MHz, DMSO-d₆) δ ppm 7.87 (d, J = 1.7 Hz, 2H), 7.76 (br d, J = 7.1 Hz, 6.81-6.74 (m, 1H), 6.70 (d, J = 14.4 Hz, 1H), 4.69 (br d, J = 2.4 Hz, 1H), 4.54-4.43 (m, 3H), 4.27 (br d, J = 2.2 Hz, 1H), 3.95-3.85 (m, 1H), 3.61 (s, 3H), 3.07-2.93 (m, 2H), 2.65-2.54 (m, 2H), 2.22 -2.13 (m, 6H), 2.04 (d, J = 17.1 Hz, 3H), 1.90-1.64 (m, 3H), 1.42 (br d, J = 2.4 Hz, 1H) |

### Example 480

### Preparation Methyl (R)-3-(4-amino-5-(3-(5-(difluoromethyl)thiophene-2-carboxamido)piperidin-1-yl)pyrrolo[2,1-f][1,2,4]triazin-7-yl)-2,5-dihydro-1H-pyrrole-1-carboxylate

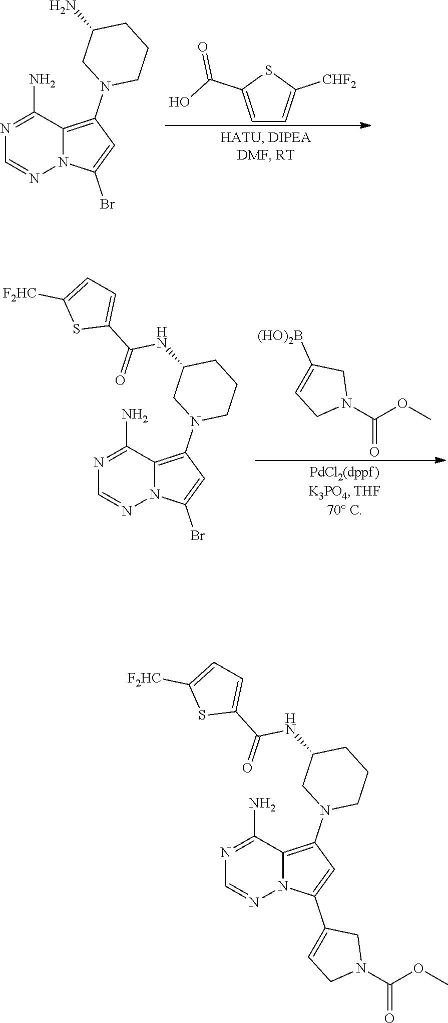 [SMILES: Nc1ncnn2c(Br)cc(N3CCC[C@@H](N)C3)c12 ; O=C(O)c1ccc(C(F)F)s1 ; Nc1ncnn2c(Br)cc(N3CCC[C@@H](NC(=O)c4ccc(C(F)F)s4)C3)c12 ; COC(=O)N1CC=C(B(O)O)C1 ; COC(=O)N1CC=C(c2cc(N3CCC[C@@H](NC(=O)c4ccc(C(F)F)s4)C3)c3c(N)ncnn23)C1]

### Example 480A. (R)—N-(1-(4-amino-7-bromopyrrolo[2,1-f][1,2,4]triazin-5-yl)piperidin-3-yl)-5-(difluoromethyl)thiophene-2-carboxamide

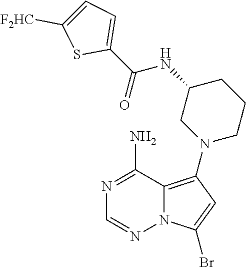 [SMILES: Nc1ncnn2c(Br)cc(N3CCC[C@@H](NC(=O)c4ccc(C(F)F)s4)C3)c12]

(R)-5-(3-aminopiperidin-1-yl)-7-bromopyrrolo[2,1-f][1,2,4]triazin-4-amine (300 mg, 0.964 mmol), 5-(difluoromethyl)thiophene-2-carboxylic acid (172 mg, 0.964 mmol), HATU (440 mg, 1.16 mmol) and DIEA (0.505 mL, 2.89 mmol) were taken in DMF (5 mL). The mixture was stirred at 25° C. for 2 h followed by the addition of water (50 mL) and ethyl acetate (40 mL). Ethyl acetate layer was separated, dried over Na₂SO₄ and concentrated. The residue was purified by silica-gel column chromatography which afforded off-white solid as Example 480A (420 mg, 92%). LC-MS Method E: RT=1.42 min, [M+H]⁺=473.1.

### Example 480

### Methyl (R)-3-(4-amino-5-(3-(5-(difluoromethyl)thiophene-2-carboxamido)piperidin-1-yl)pyrrolo[2,1-f][1,2,4]triazin-7-yl)-2,5-dihydro-1H-pyrrole-1-carboxylate

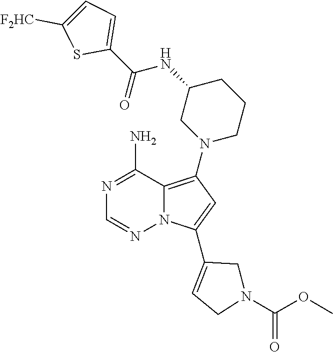 [SMILES: COC(=O)N1CC=C(c2cc(N3CCC[C@@H](NC(=O)c4ccc(C(F)F)s4)C3)c3c(N)ncnn23)C1]

Example 480A, (1-(methoxycarbonyl)-2,5-dihydro-1H-pyrrol-3-yl)boronic acid (43.5 mg, 0.255 mmol), K₃PO₄ (100 mg, 0.637 mmol) and PdCl₂(dppf) (7.8 mg, 10.1 μmol) were taken THF (2 mL)-water (1 mL). Reaction mixture was degassed and stirred at 70° C. for 2 h. The mixture was concentrated and water (20 mL) and ethyl acetate (30 mL) were added. Ethyl acetate layer was separated, dried over Na₂SO₄ and concentrated. The crude product was purified by flash chromatography to afford Example 480 (4.4 mg, 4% yield) as an off-white solid. LC-MS Method E: [M+H]⁺=518.1; ¹H NMR (400 MHz, DMSO-d₆) δ ppm 8.56 (d, J=7.8 Hz, 1H), 8.08-7.89 (m, 1H), 7.86 (s, 1H), 7.84-7.79 (m, 1H), 7.51-7.46 (m, 1H), 7.31 (t, J=56.0 Hz, 1H), 6.88 (br s, 1H), 6.76-6.69 (m, 2H), 4.60-4.48 (m, 2H), 4.35-4.24 (m, 2H), 4.18-4.07 (m, 1H), 3.66 (d, J=4.2 Hz, 3H), 3.21-3.15 (m, 1H), 3.04-2.95 (m, 1H), 2.76-2.60 (m, 2H), 1.98-1.72 (m, 3H), 1.57-1.46 (m, 1H).

The following examples in Table 38 were prepared using the procedures described for Example 479, using corresponding aromatic acids and (R)-5-(3-aminopiperidin-1-yl)-7-bromopyrrolo[2,1-f][1,2,4]triazin-4-amine followed by Suzuki coupling using appropriate boronic acids or boronate esters.

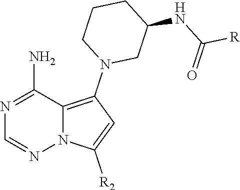

TABLE 38

| | | | | | HPLC | |
|---|---|---|---|---|---|---|
| Ex | | | | LCMS | Method, | |
| No. | R | R₂ | Name | (M + H)⁺ | RT (min.) | ¹H NMR |
| 481 | 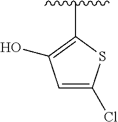 [SMILES: *c1sc(Cl)cc1O] | 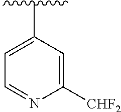 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 5-chloro-3- hydroxythiophene- 2-carboxamide | 520.1 | E: 1.404 F: 1.422 | (400 MHz, DMSO-d₆) δ ppm 12.04 (br s, 1H), 8.70 (d, J = 5.4 Hz, 1H), 8.48 (s, 1H), 8.30 (d, J = 5.1 Hz, 1H), 8.02 (s, 1H), 7.49 (s, 1H), 7.23 (s, 1H), 7.10 (s, 1H), 6.98 (s, 2H), 6.83 (s, 1H), 4.23-4.15 (m, 1H), 3.25-3.20 (m, 1H), 2.99 (br, dd, J = 1.8, 2.8 Hz, 1H), 2.90-2.76 (m, 2H), 1.94-1.75 (m, 3H), 1.61-1.50 (m, 1H) |
| 482 | 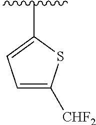 [SMILES: *c1ccc(C(F)F)s1] | 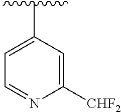 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 5- (difluoromethyl) thiophene-2- carboxamide | 520.1 | E: 1.690 | (400 MHz, DMSO-d₆) δ ppm 8.71 (d, J = 5.4 Hz, 1H), 8.60 (d, J = 7.8 Hz, 1H), 8.48 (s, 2H), 8.30 (d, J = 5.1 Hz. 1H), 8.03 (s, 1H), 7.87-7.77 (m, 1H), 7.52- 7.46 (m, 2H), 7.32 (t, J = 54.5 Hz, 2H), 6.98 (t, J = 54.0 Hz, 1H), 4.25-4.15 (m, 1H), 3.28 (br d, J = 1.0 Hz, 1H), 3.11-3.06 (m, 1H), 2.81-2.69 (m, 2H), 2.00- 1.78 (m, 3H), 1.62-1.49 (m, 1H) |
| 483 | 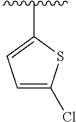 [SMILES: *c1ccc(Cl)s1] | 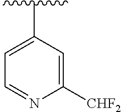 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 5-chlorothiophene- 2-carboxamide | 504.1 | F: 1.449 | (400 MHz, DMSO-d₆) δ ppm (d, J = 5.4 Hz, 1H), 8.57-8.42 (m, 3H), 8.29 (d, J = 5.1 Hz, 1H), 8.04 (s, 1H), 7.73 (d, J = 4.2 Hz, 1H), 7.48 (s, 1H), 7.38-7.14 (m, 2H), 6.98 (t, J = 54.0 Hz, 1H), 4.20-4.13 (m, 1H), 3.31-3.21 (m, 1H), 3.11-2.94 (m, 1H), 2.81-2.66 (m, 2H), 2.01-1.78 (m, 3H), 1.61- 1.46 (m, 1H) |
| 484 | 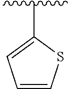 [SMILES: *c1cccs1] | 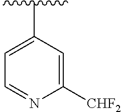 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3- yl)thiophene-2- carboxamide | 470.2 | E: 1.571 F: 1.263 | (400 MHz, DMSO-d₆) δ ppm 8.69 (d, J = 5.4 Hz, 1H), 8.50 (s, 1H), 8.38 (d, J = 7.8 Hz, 1H), 8.31 (d, J = 5.6 Hz, 1H), 8.16 (s, 1H), 7.99 (s, 1H), 7.83 (d, J = 3.9 Hz, 1H), 7.75 (d, J = 4.9 Hz, 1H), 7.67-7.49 (m, 1H), 7.46 (s, 1H), 7.18-7.12 (m, 1H), 6.97 (t, J = 55.0 Hz, 1H), 4.18 (br s, 1H), 3.27 (br s, 1H), 3.11-3.01 (m, 1H), 2.81-2.69 (m, 2H), 1.99-1.75 (m, 3H), 1.60-1.48 (m, 1H) |
| 485 | 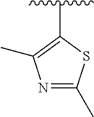 | 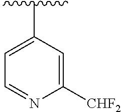 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 2,4- dimethylthiazole-5- carboxamide | 499.1 | E: 1.409 F: 1.140 | (400 MHz, DMSO-d₆) δ ppm 8.69 (d, J = 5.3 Hz, 1H), 8.50 (s, 1H), 8.32 (d, J = 5.3 Hz, 1H), 8.13 (br d, J = 7.8 Hz, 2H), 7.99 (s, 1H), 7.46 (s, 1H), 7.14-6.75 (m, 2H), 4.14 (br s, 1H), 3.22 (br d, J = 8.5 Hz, 1H), 2.98 (br s, 1H), 2.85-2.69 (m, 2H), 2.61 (s, 3H), 2.47 (s, 3H), 1.87 (br s, 3H), 1.57 (br s, 1H) |
| 486 | 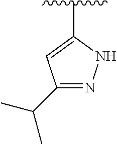 [SMILES: *c1cc(C(C)C)n[nH]1] | 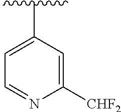 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 3-isopropyl-1H- pyrazole-5- carboxamide | 496.2 | E: 1.489 F: 1.266 | (400 MHz, DMSO-d₆) δ ppm 12.90 (br s, 1H), 8.69 (d, J = 5.1 Hz, 1H), 8.50 (s, 1H), 8.31 (d, J = 6.1 Hz, 1H), 8.16 (br s, 1H), 7.99 (s, 1H), 7.89 (s, 1H), 7.44 (s, 1H), 7.13-6.78 (m, 2H), 6.39 (s, 1H), 4.23-4.09 (m, 1H), 3.22-3.16 (m, 1H), 3.08-2.89 (m, 2H), 2.81-2.68 (m, 2H), 1.96-1.75 (m, 3H), 1.59 (br d, J = 11.7 Hz, 1H), 1.23 (d, J = 6.8 Hz, 6H) |
| 487 | 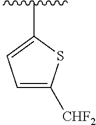 [SMILES: *c1ccc(C(F)F)s1] | 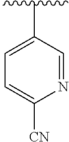 [SMILES: *c1ccc(C#N)nc1] | (R)-N-(1-(4-amino- 7-(6-cyanopyridin- 3-yl)pyrrolo[2,1- f][1,2,4]triazin-5- yl)piperidin-3-yl)- 5- (difluoromethyl)thi ophenc-2- carboxamide | 495.1 | E: 1.678 F: 1.375 | (400 MHz, DMSO-d₆) δ ppm 9.45 (d, J = 2.0 Hz, 1H), 8.83 (dd, J = 2.1, 8.2 Hz, 1H), 8.61 (d, J = 7.8 Hz, 1H), 8.20 (br d, J = 4.6 Hz, 1H), 8.10 (d, J = 8.6 Hz, 1H), 7.96 (s, 1H), 7.83 (br d, J = 2.4 Hz, 1H), 7.51-7.14 (m, 3H), 7.09-6.99 (m, 1H), 4.23- 4.16 (m, 1H), 3.24 (br d, J = 9.3 Hz, 1H), 3.09-3.02 (m, 1H), 2.79-2.69 (m, 2H), 1.99-1.79 (m, 3H), 1.61-1.49 (m, 1H) |
| 488 | 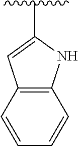 [SMILES: *c1cc2ccccc2[nH]1] | 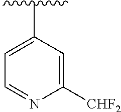 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 1H-indole-2- carboxamide | 503.2 | E: 1.790 F: 1.515 | (400 MHz, DMSO-d₆) δ ppm 11.53 (d, J = 1.3 Hz, 1H), 8.68 (d, J = 5.3 Hz, 1H), 8.50 (d, J = 1.0 Hz, 1H), 8.35 (d, J = 8.0 Hz, 1H), 8.31 (td, J = 0.8, 5.3 Hz, 1H), 8.23- 8.10 (m, 1H), 7.99 (s, 1H), 7.61 (dd, J = 0.9, 7.9 Hz, 1H), 7.47 (s, 1H), 7.42 (dd, J = 0.8, 8.3 Hz, 1H), 7.22- 7.13 (m, 2H), 7.11-6.79 (m, 3H), 4.28-4.17 (m, 1H), 3.30-3.24 (m, 1H), 3.11-3.03 (m, 1H), 2.86-2.70 (m, 2H), 2.01-1.81 (m, 3H), 1.67- 1.51 (m, 1H) |
| 489 | 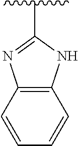 [SMILES: *c1nc2ccccc2[nH]1] | 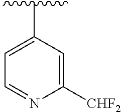 [SMILES: *c1ccnc(C(F)F)c1] | (R)-N-(1-(4-amino- 7-(2- (difluoromethyl) pyridin-4- yl)pyrrolo[2,1- f][1,2,4]triazin-5- yl)piperidin-3-yl)- 1H- benzo[d]imidazole- 2-carboxamide | 504.2 | E: 1.638 F: 1.308 | (400 MHz, DMSO-d₆) δ ppm 8.95 (d, J = 8.3 Hz, 1H), 8.71 (d, J = 5.4 Hz, 1H), 8.49 (s, 2H), 8.31 (d, J = 5.4 Hz, 1H), 8.05 (s, 1H), 7.64 (dd, J = 3.3, 6.0 Hz, 2H), 7.49 (s, 1H), 7.34-7.23 (m, 3H), 6.98 (t, J = 56.2 Hz, 2H), 4.34-4.28 (m, 1H), 3.26 (br d, J = 7.3 Hz, 1H), 3.12-3.06 (m, 1H), 2.92-2.85 (m, 1H), 2.80-2.71 (m, 1H), 1.96-1.85 (m, 3H), 1.81- 1.65 (m, 1H) |
| 490 | 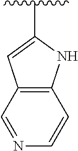 [SMILES: *c1cc2cnccc2[nH]1] | 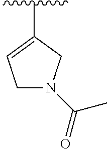 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 1H-pyrrolo[3,2- c]pyridine-2- carboxamide | 486.3 | F: 0.750 | (400 MHz, DMSO-d₆) δ ppm 12.00 (br s, 1H), 8.94 (s, 1H), 8.52 (d, J = 7.3 Hz, 1H), 8.22 (d, J = 5.9 Hz, 1H), 8.08-7.90 (m, 1H), 7.88 (d, J = 1.5 Hz, 1H), 7.41-7.29 (m, 2H), 6.96 (s, 1H), 6.81- 6.68 (m, 2H), 4.71-4.66 (m, 1H), 4.49 (br d, J = 10.8 Hz, 2H), 4.28-4.17 (m, 2H), 3.24-3.18 (m, 1H), 3.06-3.00 (m, 1H), 2.76-2.62 (m, 2H), 2.06-1.75 (m, 6H), 1.64- 1.49 (m, 1H) |
| 491 | 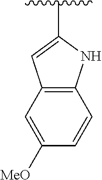 [SMILES: *c1cc2cc(OC)ccc2[nH]1] | 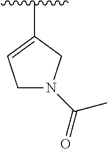 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 5-methoxy-1H- indole-2- carboxamide | 515.2 | E: 1.350 F: 1.145 | (400 MHz, DMSO-d₆) δ ppm 11.39 (br s, 1H), 8.35- 8.23 (m, 1H), 7.88 (d, J = 1.5 Hz, 2H), 7.31 (d, J = 8.8 Hz, 1H), 7.15-7.03 (m, 2H), 6.96-6.63 (m, 4H), 4.75- 4.65 (m, 1H), 4.57-4.42 (m, 2H), 4.34-4.13 (m, 2H), 3.76 (s, 3H), 3.26-3.20 (m, 1H), 3.10-3.00 (m, 1H), 2.79-2.68 (m, 2H), 2.03 (d, J = 15.4 Hz, 3H), 1.95-1.76 (m, 3H), 1.60-1.48 (m, 1H) |
| 492 | 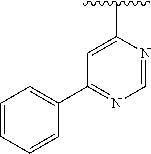 [SMILES: *c1cc(-c2ccccc2)ncn1] | 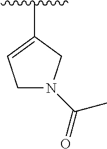 [SMILES: *C1=CCN(C(C)=O)C1] | (R)-N-(1-(7-(1- acetyl-2,5-dihydro- 1H-pyrrol-3-yl)-4- aminopyrrolo [2,1- f][1,2,4]triazin-5- yl)piperidin-3-yl)- 6- phenylpyrimidine- 4-carboxamide | 524.2 | E: 1.592 F: 1.299 | (400 MHz, DMSO-d₆) δ ppm 9.39 (d, J = 0.7 Hz, 1H), 8.94 (br dd, J = 2.6, 4.3 Hz, 1H), 8.48 (d, J = 1.0 Hz, 1H), 8.35-8.22 (m, 3H), 7.92 (s, 1H), 7.65-7.53 (m, 3H), 6.81-6.71 (m, 2H), 4.70 (br d, J = 2.0 Hz, 1H), 4.54-4.43 (m, 2H), 4.33-4.22 (m, 2H), 3.21-3.16 (m, 1H), 3.07-3.00 (m, 1H), 2.90- 2.83 (m, 1H), 2.77 (br d, J = 4.6 Hz, 1H), 2.03 (d, J = 16.1 Hz, 3H), 1.94-1.82 (m, 3H), 1.76-1.64 (m, 1H) |
| 493 | 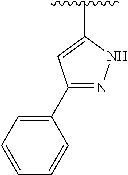 [SMILES: *c1cc(-c2ccccc2)n[nH]1] | 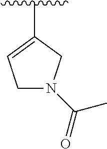 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 3-phenyl-1H- pyrazole-5- carboxamide | 512.2 | E: 1.357 | (400 MHz, DMSO-d6) δ ppm 8.16 (br s, 1H), 7.93 (s, 1H), 7.80 (d, J = 7.5 Hz, 2H), 7.52-7.40 (m, 3H), 7.38- 7.32 (m, 1H), 7.17 (br s, 2H), 6.84-6.67 (m, 2H), 4.70 (br s, 1H), 4.50 (br d, J = 13.0 Hz, 2H), 4.27 (br d, J = 2.3 Hz, 1H), 4.25-4.12 (m, 1H), 3.21 (br d, J = 5.0 Hz, 1H), 3.05-3.00 (m, 1H), 2.72 (br d, J = 10.3 Hz, 2H), 2.03 (d, J = 15.8 Hz, 3H), 1.86 (br d, J = 6.3 Hz, 3H), 1.60 (br s, 1H) |
| 494 | 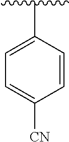 [SMILES: *c1ccc(C#N)cc1] | 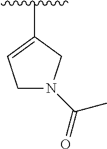 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 4-cyanobenzamide | 471.2 | E: 1.248 | (400 MHz, DMSO-d6) δ ppm 8.63 (d, J = 7.5 Hz, 2H), 8.03-7.83 (m, 5H), 7.32 (br s, 1H), 6.88-6.68 (m, 2H), 4.70 (br s, 1H), 4.50 (br d, J = 10.5 Hz, 2H), 4.32-4.14 (m, 2H), 3.25-3.20 (m, 1H), 3.04 (br s, 1H), 2.76-2.68 (m, 2H), 2.03 (d, J = 15.5 Hz, 3H), 1.86 (br s, 3H), 1.56 (br s, 1H) |
| 495 | 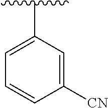 [SMILES: *c1cccc(C#N)c1] | 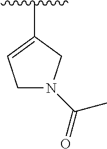 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 3-cyanobenzamide | 471.1 | E: 1.216 F: 0.988 | (400 MHz, DMSO-d₆) δ ppm 8.57 (d, J = 8.0 Hz, 1H), 8.29 (d, J = 1.5 Hz, 1H), 8.19- 8.13 (m, 1H), 8.07-7.97 (m, 1H), 7.88 (d, J = 1.5 Hz, 1H), 7.74-7.64 (m, 1H), 6.83-6.64 (m, 2H), 4.69 (br d, J = 1.5 Hz, 1H), 4.48 (br d, J = 9.0 Hz, 2H), 4.29-4.25 (m, 1H), 4.23-4.11 (m, 1H), 3.19 (br dd, J = 3.3, 8.3 Hz, 1H), 3.06-2.94 (m, 1H), 2.76-2.69 (m, 1H), 2.60 (br d, J = 4.5 Hz, 1H), 2.03 (d, J = 16.1 Hz, 3H), 1.96-1.71 (m, 3H), 1.60-1.47 (m, 1H) |
| 496 | 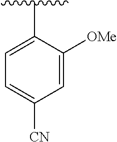 [SMILES: *c1ccc(C#N)cc1OC] | 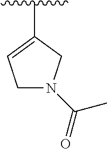 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 4-cyano-2- methoxybenzamide | 501.3 | E: 1.354 F: 1.353 | (400 MHz, DMSO-d₆) δ ppm 8.37 (s, 1H), 7.88 (d, J = 1.5 Hz, 1H), 7.69 (d, J = 7.6 Hz, 1H), 7.64 (s, 1H), 7.58 (s, 1H), 7.52-7.43 (m, 2H), 7.38-7.29 (m, 1H), 6.80- 6.67 (m, 1H), 4.70 (br d, J = 1.5 Hz, 1H), 4.53-4.45 (m, 2H), 3.85 (m, 2H), 3.90 (s, 3H), 3.49 (d, J = 6.8 Hz, 1H), 3.18 (s, 1H), 2.95-2.71 (m, 3H), 2.04 (d, J = 16.9 Hz, 3H), 1.90-1.69 (m, 3H) |
| 497 | 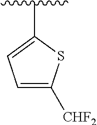 [SMILES: *c1ccc(C(F)F)s1] | 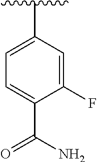 [SMILES: *c1ccc(C(N)=O)c(F)c1] | (R)-N-(1-(4-amino- 7-(4-carbamoyl-3- fluorophenyl)pyrrol o[2,1- f][1,2,4]triazin-5- yl)piperidin-3-yl)- 5- (difluoromethyl) thiophene-2- carboxamide | 530.1 | E: 1.447 F: 1.189 | (400 MHz, DMSO-d₆) δ ppm 8.60 (d, J = 7.3 Hz, 1H), 8.14 (d, J = 13.0 Hz, 1H), 8.02 (d, J = 8.1 Hz, 1H), 7.93 (s, 1H), 7.86-7.83 (m, 1H), 7.77-7.71 (m, 1H), 7.65 (br d, J = 13.0 Hz, 2H), 7.49 (br d, J = 2.4 Hz, 1H), 7.34- 7.17 (m, 2H), 4.20-4.16 (m, 1H), 3.25-3.19 (m, 1H), 3.08-3.04 (m, 1H), 2.73 (br dd, J = 2.1, 9.9 Hz, 2H), 1.97-1.84 (m, 3H), 1.57 (br d, J = 3.2 Hz, 1H) |
| 498 | 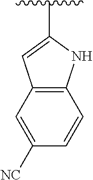 [SMILES: *c1cc2cc(C#N)ccc2[nH]1] | 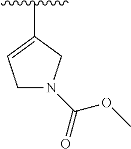 [SMILES: *C1=CCN(C(=O)OC)C1] | Methyl (R)-3-(4- amino-5-(3-(5- cyano-1H-indole-2- carboxamido) piperidin-1- yl)pyrrolo[2,1- f][1,2,4]triazin-7- yl)-2,5-dihydro- 1H-pyrrole-1- carboxylate | 526.1 | E: 1.661 F: 1.347 | (400 MHz, DMSO-d₆) δ ppm 12.15 (s, 1H), 8.57 (br d, J = 7.6 Hz, 2H), 8.26 (s, 1H), 7.95 (s, 1H), 7.62-7.47 (m, 2H), 7.37 (s, 2H), 6.84 (d, J = 5.9 Hz, 1H), 6.77 (br d, J = 1.7 Hz, 1H), 4.61-4.47 (m, 2H), 4.36-4.20 (m, 3H), 3.66 (d, J = 3.2 Hz, 3H), 3.24 (br dd, J = 2.4, 9.0 Hz, 1H), 3.09-3.03 (m, 1H), 2.80- 2.67 (m, 2H), 2.02-1.78 (m, 3H), 1.64-1.46 (m, 1H) |
| 499 | 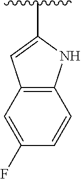 [SMILES: *c1cc2cc(F)ccc2[nH]1] | 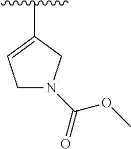 [SMILES: *C1=CCN(C(=O)OC)C1] | methyl (R)-3-(4- amino-5-(3-(5- fluoro-1H-indole-2- carboxamido) piperidin-1- yl)pyrrolo[2,1- f][1,2,4]triazin-7- yl)-2,5-dihydro- 1H-pyrrole-1- carboxylate | 519.2 | E: 1.733 F: 1.450 | (400 MHz, DMSO-d₆) δ ppm 11.65 (s, 1H), 8.38 (br d, J = 7.5 Hz, 1H), 7.86 (s, 1H), 7.46-7.37 (m, 2H), 7.19 (s, 1H), 7.04 (dt, J = 2.3, 9.1 Hz, 1H), 6.81-6.69 (m, 2H), 4.54 (br d, J = 12.0 Hz, 2H), 4.36-4.16 (m, 3H), 3.65 (d, J = 3.0 Hz, 3H), 3.23-3.17 (m, 1H), 3.05-2.96 (m, 1H), 1.98-1.71 (m, 4H), 1.61-1.47 (m, 1H), 1.20-1.10 (m, 2H) |
| 500 | 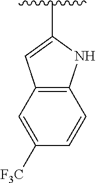 [SMILES: *c1cc2cc(C(F)(F)F)ccc2[nH]1] | 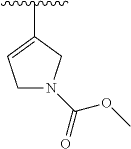 [SMILES: *C1=CCN(C(=O)OC)C1] | methyl (R)-3-(4- amino-5-(3-(6- (trifluoromethyl)- 1H-indole-2- carboxamido) piperidin-1- yl)pyrrolo[2,1- f][1,2,4]triazin-7- yl)-2,5-dihydro- 1H-pyrrole-1- carboxylate | 569.2 | E: 2.005 F: 1.688 | 1H NMR (400 MHz, DMSO- d₆) δ ppm 12.01 (s, 1H), 8.52 (d, J = 8.0 Hz, 1H), 8.08 (s, 2H), 7.87 (s, 1H), 7.61 (d, J = 8.5 Hz, 1H), 7.46 (dd, J = 1.5, 8.5 Hz, 1H), 7.37 (d, J = 1.5 Hz, 1H), 6.95 (br s, 1H), 6.78-6.63 (m, 2H), 4.61-4.46 (m, 2H), 4.37-4.15 (m, 3H), 3.66 (d, J = 3.5 Hz, 3H), 3.22 (br d, J = 12.0 Hz, 1H), 3.07-3.00 (m, 1H), 2.80-2.69 (m, 2H), 2.01- 1.76 (m, 3H), 1.65-1.49 (m, 1H) |
| 501 | 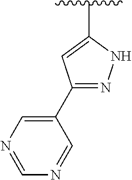 [SMILES: *c1cc(-c2cncnc2)n[nH]1] | 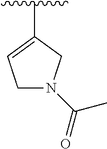 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 3-(pyrimidin-5-yl)- 1H-pyrazole-5- carboxamide | 514.0 | E: 1.063 F: 0.855 | (400 MHz, DMSO-d₆) δ ppm 13.93 (s, 1H), 9.16 (s, 3H), 8.48 (s, 1H), 8.15 (br s, 1H), 7.87 (d, J = 1.5 Hz, 1H), 7.46 (br s, 1H), 6.83-6.59 (m, 2H), 4.73-4.64 (m, 1H), 4.54-4.43 (m, 2H), 4.33- 4.08 (m, 2H), 3.20-3.06 (m, 2H), 3.03-2.89 (m, 2H), 2.78-2.70 (m, 1H), 2.02 (d, J = 16.3 Hz, 3H), 1.89 (br s, 2H), 1.55 (br s, 1H) |
| 502 | 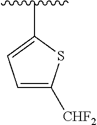 [SMILES: *c1ccc(C(F)F)s1] | 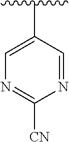 [SMILES: *c1cnc(C#N)nc1] | (R)-N-(1-(4-amino- 7-(2- cyanopyrimidin-5- yl)pyrrolo [2,1- f][1,2,4]triazin-5- yl)piperidin-3-yl)- 5- (difluoromethyl) thiophene-2- carboxamide | 496.1 | E: 1.829 F: 1.521 | |
| 503 | 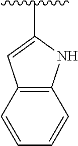 [SMILES: *c1cc2ccccc2[nH]1] | 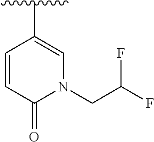 [SMILES: *c1ccc(=O)n(CC(F)F)c1] | (R)-N-(1-(4-amino- 7-(1-(2,2- difluoroethyl)-6- oxo-1,6- dihydropyridin-3- yl)pyrrolo[2,1- f][1,2,4]triazin-5- yl)piperidin-3-yl)- 1H-indole-2- carboxamide | 533.3 | E: 1.622 F: 1.364 | (400 MHz, DMSO-d₆) δ ppm 11.53 (d, J = 1.3 Hz, 1H), 8.50 (d, J = 2.0 Hz, 1H), 8.34 (d, J = 7.8 Hz, 1H), 8.12 (dd, J = 2.5, 9.5 Hz, 1H), 7.86 (s, 1H), 7.61 (d, J = 8.5 Hz, 1H), 7.42 (d, J = 8.3 Hz, 1H), 7.24-7.14 (m, 2H), 7.07-7.00 (m, 1H), 6.95 (s, 1H), 6.57 (d, J = 9.5 Hz, 1H), 6.51-6.19 (m, 1H), 4.45 (dt, J = 3.8, 14.5 Hz, 2H), 4.24 (br s, 1H), 3.25 (br s, 2H), 3.07 (br dd, J = 1.6, 9.6 Hz, 1H), 2.72 (ddd, J = 1.4, 3.5, 4.9 Hz, 2H), 2.00-1.80 (m, 3H), 1.58 (br s, 1H) |
| 504 | 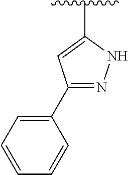 [SMILES: *c1cc(-c2ccccc2)n[nH]1] | 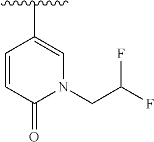 [SMILES: *c1ccc(=O)n(CC(F)F)c1] | (R)-N-(1-(4-amino- 7-(1-(2,2- difluoroethyl)-6- oxo-1,6- dihydropyridin-3- yl)pyrrolo[2,1- f][1,2,4]triazin-5- yl)piperidin-3-yl)- 3-phenyl-1H- pyrazole-5- carboxamide | 560.2 | E: 1.535 F: 1.334 | (400 MHz, DMSO-d₆) δ ppm 8.50 (d, J = 2.5 Hz, 1H), 8.19 (br s, 1H), 8.12 (dd, J = 2.6, 9.6 Hz, 1H), 7.92 (s, 1H), 7.81-7.74 (m, 2H), 7.50- 7.41 (m, 2H), 7.40-7.31 (m, 1H), 7.20-7.10 (m, 1H), 6.97 (s, 1H), 6.58 (d, J = 9.8 Hz, 1H), 6.52-6.18 (m, 1H), 4.52-4.38 (m, 2H), 4.30-4.16 (m, 1H), 3.26-3.23 (m, 2H), 3.11-3.05 (m, 2H), 2.81-2.70 (m, 2H), 1.99- 1.79 (m, 3H), 1.60 (br dd, J = 1.3, 3.5 Hz, 1H) |
| 505 | 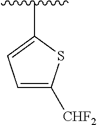 [SMILES: *c1ccc(C(F)F)s1] | 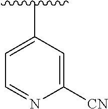 [SMILES: *c1ccnc(C#N)c1] | (R)-N-(1-(4-amino- 7-(2-cyanopyridin- 4-yl)pyrrolo[2,1- f][1,2,4]triazin-5- yl)piperidin-3-yl)- 5- (difluoromethyl) thiophene-2- carboxamide | 495.2 | E: 1.756 F: 1.459 | (400 MHz, DMSO-d₆) δ ppm 8.78-8.69 (m, 2H), 8.59 (d, J = 7.8 Hz, 1H), 8.56-8.48 (m, 1H), 8.24 (br s, 1H), 8.01 (s, 1H), 7.85-7.78 (m, 1H), 7.53 (s, 1H), 7.51-7.46 (m, 1H), 7.31 (t, ) = 57.5 Hz, 1H), 7.05 (br s, 1H), 4.17 (br s, 1H), 3.27-3.18 (m, 1H), 3.09-3.01 (m, 1H), 2.78- 2.65 (m, 2H), 1.99-1.80 (m, 3H), 1.62-1.49 (m, 1H) |
| 506 | 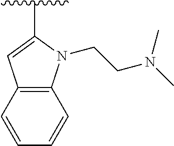 [SMILES: *c1cc2ccccc2n1CCN(C)C] | 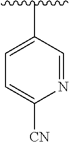 [SMILES: *c1ccc(C#N)nc1] | (R)-N-(1-(4-amino- 7-(6-cyanopyridin- 3-yl)pyrrolo[2,1- f][1,2,4]triazin-5- yl)piperidin-3-yl)- 1-(2- (dimethylamino) ethyl)-1H-indole-2- carboxamide | 549.2 | E: 1.662 F: 1.227 | (400 MHz, DMSO-d6) δ ppm 9.66 (br s, 1H), 9.44 (dd, J = 0.8, 2.3 Hz, 1H), 8.82 (dd, J = 2.3, 8.5 Hz, 1H), 8.64 (br d, J = 8.3 Hz, 1H), 8.23 (br s, 1H), 8.10 (dd, J = 0.8, 8.5 Hz, 1H), 7.97 (s, 1H), 7.70 (d, J = 7.8 Hz, 1H), 7.64 (d, J = 8.5 Hz, 1H), 7.44 (s, 1H), 7.39-7.32 (m, 1H), 7.30 (s, 1H), 7.23-6.93 (m, 2H), 4.80 (br t, J = 7.1 Hz, 2H), 4.24 (br s, 1H), 3.28-3.25 (m, 1H), 3.10 (dq, ) = 4.8, 7.3 Hz, 3H), 2.96-2.85 (m, 7H), 2.79 (br s, 1H), 1.92 (br s, 3H), 1.61 (br s, 1H) |
| 507 | 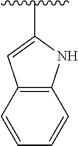 [SMILES: *c1cc2ccccc2[nH]1] | 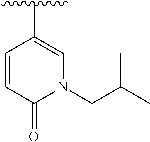 [SMILES: *c1ccc(=O)n(CC(C)C)c1] | (R)-N-(1-(4-amino- 7-(1-isobutyl-6- ox0-1,6- dihydropyridin-3- yl)pyrrolo [2,1- f][1,2,4]triazin-5- yl)piperidin-3-yl)- 1H-indole-2- carboxamide | 525.2 | E: 1.670 F: 1.429 | (400 MHz, DMSO-d6) δ ppm 11.54 (s, 1H), 8.44 (d, J = 1.3 Hz, 1H), 8.37-8.30 (m, 1H), 8.11-8.02 (m, 1H), 7.92-7.86 (m, 1H), 7.62 (d, J = 7.5 Hz, 1H), 7.43 (dd, J = 0.9, 7.6 Hz, 1H), 7.21-7.14 (m, 3H), 7.06-7.02 (m, 2H), 6.99 (s, 1H), 6.51 (d, J = 10.0 Hz, 1H), 4.27 (s, 1H), 3.80-3.77 (m, 2H), 3.12- 3.08 (m, 2H), 2.77-2.69 (m, 3H), 2.08 (s, 2H), 1.90 (br s, 2H), 1.58 (s, 1H), 0.89 (d, J = 6.5 Hz, 6H) |
| 508 | 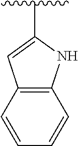 [SMILES: *c1cc2ccccc2[nH]1] | 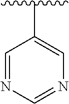 [SMILES: *c1cncnc1] | (R)-N-(1-(4-amino- 7-(pyrimidin-5- yl)pyrrolo[2,1- f][1,2,4]triazin-5- yl)piperidin-3-yl)- 1H-indole-2- carboxamide | 454.2 | E: 1.458 F: 1.214 | (400 MHz, DMSO-d₆) δ ppm 11.53 (s, 1H), 9.46 (s, 2H), 9.10 (s, 1H), 8.36 (d, J = 7.8 Hz, 2H), 7.95 (s, 1H), 7.61 (d, J = 8.3 Hz, 1H), 7.46- 7.39 (m, 1H), 7.34 (s, 1H), 7.23-7.13 (m, 3H), 7.03 (t, J = 8.1 Hz, 1H), 4.26 (br s, 1H), 3.27 (br d, J = 5.0 Hz, 1H), 3.12-3.05 (m, 1H), 2.81-2.69 (m, 2H), 1.90 (br s, 3H), 1.58 (br s, 1H) |
| 509 | 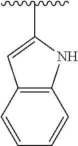 [SMILES: *c1cc2ccccc2[nH]1] | 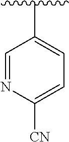 [SMILES: *c1ccc(C#N)nc1] | (R)-N-(1-(4-amino- 7-(6-cyanopyridin- 3-yl)pyrrolo[2,1- f][1,2,4]triazin-5- yl)piperidin-3-yl)- 1H-indole-2- carboxamide | 478.1 | E: 1.742 F: 1.455 | (400 MHz, DMSO-d₆) δ ppm 11.53 (d, J = 1.3 Hz, 1H), 9.44 (d, J = 2.0 Hz, 1H), 8.81 (dd, J = 2.3, 8.3 Hz, 1H), 8.36 (d, J = 8.0 Hz, 1H), 8.25 (br s, 1H), 8.09 (d, J = 8.5 Hz, 1H), 7.97 (s, 1H), 7.61 (d, J = 7.8 Hz, 1H), 7,47- 7.38 (m, 2H), 7.26-7.06 (m, 3H), 7.06-6.99 (m, 1H), 4.24 (br s, 1H), 3.28-3.24 (m, 1H), 3.11-3.04 (m, 1H), 2.76 (br s, 2H), 1.90 (br s, 3H), 1.58 (br s, 1H) |
| 510 | 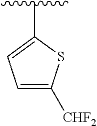 [SMILES: *c1ccc(C(F)F)s1] | 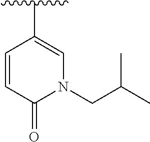 [SMILES: *c1ccc(=O)n(CC(C)C)c1] | (R)-N-(1-(4-amino- 7-(1-isobutyl-6- oxo-1,6- dihydropyridin-3- yl)pyrrolo [2,1- f][1,2,4]triazin-5- yl)piperidin-3-yl)- 5- (difluoromethyl) thiophene-2- carboxamide | 542.2 | E: 1.644 F: 1.379 | (400 MHz, DMSO-d6) δ ppm 8.57 (d, J = 7.5 Hz, 1H), 8.43 (d, J = 2.5 Hz, 1H), 8.04 (dd, J = 2.8, 9.5 Hz, 1H), 7.84-7.75 (m, 2H), 7.51-7.13 (m, 4H), 6.91 (s, 1H), 6.48 (d, J = 9.5 Hz, 1H), 4.20- 4.09 (m, 1H), 3.77 (d, J = 7.3 Hz, 2H), 3.23-3.17 (m, 1H), 3.08-2.99 (m, 1H), 2.75-2.68 (m, 1H), 2.14-2.07 (m, 1H), 1.99-1.75 (m, 3H), 1.68-1.45 (m, 2H), 0.89 (d, J = 6.8 Hz, 6H) |
| 511 | 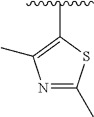 | 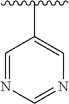 [SMILES: *c1cncnc1] | (R)-N-(1-(4-amino- 7-(pyrimidin-5- yl)pyrrolo [2,1- f][1,2,4]triazin-5- yl)piperidin-3-yl)- 2,4- dimethylthiazole-5- carboxamide | 450.1 | E: 1.168 F: 0.890 | (400 MHz, DMSO-d₆) δ ppm 9.47 (s, 2H), 9.08 (s, 1H), 8.12 (br d, J = 6.8 Hz, 2H), 7.91 (s, 1H), 7.30 (s, 1H), 6.97 (s, 1H), 4.14 (br s, 1H), 3.23-3.17 (m, 1H), 2.98 (br s, 1H), 2.81-2.71 (m, 2H), 2.61 (s, 3H), 2.47 (s, 3H), 1.87 (br s, 3H), 1.55 (br s, 1H) |
| 512 | 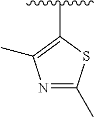 | 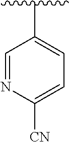 [SMILES: *c1ccc(C#N)nc1] | (R)-N-(1-(4-amino- 7-(6-cyanopyridin- 3-yl)pyrrolo[2,1- f][1,2,4]triazin-5- yl)piperidin-3-yl)- 2.4- dimethylthiazole-5- carboxamide | 474.2 | E: 1.473 F: 1.133 | (400 MHz, DMSO-d₆) δ ppm 9.45 (d, J = 1.5 Hz, 1H), 8.82 (dd, J = 2.1, 8.4 Hz, 1H), 8.09 (d, J = 8.5 Hz, 3H), 7.96 (s, 1H), 7.41 (s, 1H), 7.05 (br s, 1H), 4.20-4.09 (m, 1H), 3.22 (br d, J = 9.0 Hz, 1H), 3.07-2.90 (m, 1H), 2.74 (br s, 2H), 2.61 (s, 3H), 2.47 (s, 3H), 1.80 (br s, 3H), 1.56 (br s, 1H) |
| 513 | 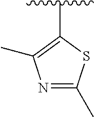 | 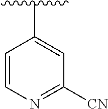 [SMILES: *c1ccnc(C#N)c1] | (R)-N-(1-(4-amino- 7-(2-cyanopyridin- 4-yl)pyrrolo[2,1- f][1,2,4]triazin-5- yl)piperidin-3-yl)- 2.4- dimethylthiazole-5- carboxamide | 474.1 | E: 1.476 F: 1.152 | (400 MHz, DMSO-d6) δ ppm 8.81-8.70 (m, 2H), 8.54 (dd, J = 1.7, 5.4 Hz, 1H), 8.16 (br dd, J = 2.4, 4.9 Hz, 2H), 8.02 (s, 1H), 7.54 (s, 1H), 7.06 (br s, 1H), 4.22- 4.09 (m, 1H), 3.25-3.17 (m, 1H), 2.96 (br s, 1H), 2.81- 2.67 (m, 2H), 2.61 (s, 3H), 2.47 (s, 3H), 1.95-1.73 (m, 3H), 1.56 (br s, 1H) |
| 514 | 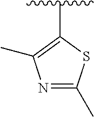 | 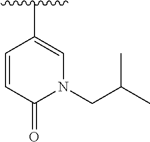 [SMILES: *c1ccc(=O)n(CC(C)C)c1] | (R)-N-(1-(4-amino- 7-(1-isobutyl-6- oxo-1,6- dihydropyridin-3- yl)pyrrolo [2,1- f][1,2,4]triazin-5- yl)piperidin-3-yl)- 2,4- dimethylthiazole-5- carboxamide | 521.2 | E: 1.420 F: 1.140 | (400 MHz, DMSO-d₆) δ ppm 8.44 (d, J = 2.3 Hz, 1H), 8.11 (br d, J = 8.0 Hz, 1H), 8.05 (dd, J = 2.6, 9.4 Hz, 2H), 7.83 (s, 1H), 6.92 (s, 2H), 6.49 (d, J = 9.5 Hz, 1H), 4.17-4.07 (m, 1H), 3.79 (d, J = 7.3 Hz, 2H), 3.21-3.18 (m, 1H), 3.01-2.94 (m, 1H), 2.81- 2.66 (m, 2H), 2.61 (s, 3H), 2.47 (s, 3H), 2.16-2.08 (m, 1H), 1.94-1.71 (m, 3H), 1.55 (br s, 1H), 0.90 (d, J = 6.5 Hz, 6H) |
| 515 | 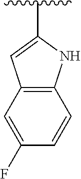 [SMILES: *c1cc2cc(F)ccc2[nH]1] | 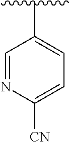 [SMILES: *c1ccc(C#N)nc1] | (R)-N-(1-(4-amino- 7-(6-cyanopyridin- 3-yl)pyrrolo[2,1- f][1,2,4]triazin-5- yl)piperidin-3-yl)- 5-fluoro-1H-indole- 2-carboxamide | 496.1 | E: 1.789 F: 1.504 | (400 MHz, DMSO-d₆) δ ppm 11.65 (d, J = 1.3 Hz, 1H), 9.43 (d, J = 1.8 Hz, 1H), 8.80 (dd, J = 2.1, 8.4 Hz, 1H), 8.42 (br d, J = 7.8 Hz, 2H), 8.10 (d, J = 8.5 Hz, 1H), 7.99 (s, 1H), 7.47-7.34 (m, 3H), 7.19 (d, J = 1.8 Hz, 2H), 7.06-6.98 (m, 1H), 4.27 (br s, 1H), 3.29-3.26 (m, 1H), 3.09 (br s, 1H), 2.73 (br s, 2H), 1.90 (br s, 3H), 1.59 (br s, 1H) |
| 516 | 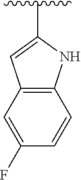 [SMILES: *c1cc2cc(F)ccc2[nH]1] | 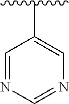 [SMILES: *c1cncnc1] | (R)-N-(1-(4-amino- 7-(pyrimidin-5- yl)pyrrolo[2,1- f][1,2,4]triazin-5- yl)piperidin-3-yl)- 5-fluoro-1H-indole- 2-carboxamide | 472.1 | E: 1.518 F: 1.270 | (400 MHz, DMSO-d₆) δ ppm 11.65 (s, 1H), 9.47 (s, 2H), 9.08 (s, 1H), 8.41 (d, J = 7.8 Hz, 1H), 8.13 (br s, 1H), 7.92 (s, 1H), 7.45-7.35 (m, 2H), 7.32 (s, 1H), 7.19 (d, J = 1.5 Hz, 1H), 7.03 (dt, J = 2.6, 9.2 Hz, 2H), 4.32- 4.19 (m, 1H), 3.28-3.24 (m, 1H), 3.12-3.02 (m, 1H), 2.82-2.68 (m, 2H), 2.04-1.80 (m, 3H), 1.64-1.47 (m, 1H) |
| 517 | 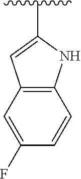 [SMILES: *c1cc2cc(F)ccc2[nH]1] | 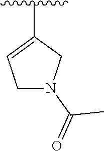 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 5-fluoro-1H-indole- 2-carboxamide | 503.2 | E: 1.497 F: 1.263 | (400 MHz, DMSO-d₆) δ ppm 11.65 (s, 1H), 8.39 (d, J = 7.8 Hz, 1H), 8.12 (s, 1H), 7.89 (s, 1H), 7.62 (s, 1H), 7.46-7.36 (m, 2H), 7.19 (s, 1H), 7.10-6.88 (m, 2H), 6.81-6.68 (m, 2H), 4.75-4.65 (m, 1H), 4.49 (br dd, J = 1.6, 13.4 Hz, 2H), 4.26 (br s, 2H), 3.25-3.20 (m, 1H), 3.08-3.00 (m, 1H), 2.77-2.67 (m, 2H), 2.03 (d, J = 15.5 Hz, 3H), 1.88 (br d, J = 1.5 Hz, 3H), 1.56 (br s, 1H) |
| 518 | 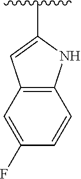 [SMILES: *c1cc2cc(F)ccc2[nH]1] | 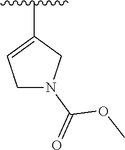 [SMILES: *C1=CCN(C(=O)OC)C1] | Methyl (R)-3-(4- amino-5-(3-(5- fluoro-1H-indole-2- carboxamido) piperidin-1- yl)pyrrolo[2,1- f][1,2,4]triazin-7- yl)-2,5-dihydro- 1H-pyrrole-1- carboxylate | 519.2 | E: 1.736 F: 1.454 | (400 MHz, DMSO-d₆) δ ppm 11.65 (s, 1H), 8.38 (d, J = 8.3 Hz, 1H), 7.85 (s, 1H), 7.47- 7.35 (m, 2H), 7.18 (d, J = 1.3 Hz, 1H), 7.03 (dt, J = 2.6, 9.2 Hz, 2H), 6.78-6.68 (m, 2H), 4.58-4.48 (m, 1H), 4.29 (br dd, J = 3.0, 4.5 Hz, 2H), 3.65 (d, J = 3.5 Hz, 2H), 3.22-3.18 (m, 2H), 3.04- 2.99 (m, 1H), 2.74-2.67 (m, 1H), 2.01-1.72 (m, 2H), 1.62-1.39 (m, 3H), 0.99-0.83 (m, 2H) |
| 519 | 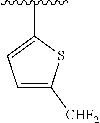 [SMILES: *c1ccc(C(F)F)s1] | 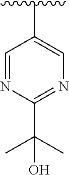 [SMILES: *c1cnc(C(C)(C)O)nc1] | (R)-N-(1-(4-amino- 7-12-(2- hydroxypropan-2- yl)pyrimidin-5- yl)pyrrolo[2,1- f][1,2,4]triazin-5- yl)piperidin-3-yl)- 5- (difluoromethyl) thiophene-2- carboxamide | 528.2 | E: 1.601 F: 1.283 | (400 MHz, DMSO-d₆) δ ppm 9.42 (br s, 2H), 8.61 (d, J = 7.3 Hz, 1H), 7.90 (br s, 2H), 7.85-7.79 (m, 1H), 7.67-6.91 (m, 4H), 4.23-4.15 (m, 1H), 3.27-3.20 (m, 2H), 3.07 (br dd, J = 3.3, 11.6 Hz, 1H), 2.82-2.70 (m, 2H), 2.01-1.79 (m, 4H), 1.65- 1.40 (m, 6H) |
| 520 | 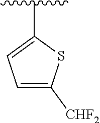 [SMILES: *c1ccc(C(F)F)s1] | 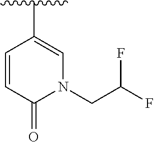 [SMILES: *c1ccc(=O)n(CC(F)F)c1] | (R)-N-(1-(4-amino- 7-(1-(2,2- difluoroethyl)-6- oxo-1,6- dihydropyridin-3- yl)pyrrolo [2,1- f][1,2,4]triazin-5- yl)piperidin-3-yl)- 5-chlorothiophene- 2-carboxamide | 550.1 | E: 1.554 F: 1.283 | (400 MHz, DMSO-d₆) δ ppm 8.49 (d, J = 2.5 Hz, 1H), 8.46 (d, J = 8.0 Hz, 1H), 8.11 (dd, J = 2.6, 9.6 Hz, 1H), 7.90 (s, 1H), 7.71 (d. J = 4.0 Hz, 1H), 7.18 (d, J = 4.3 Hz, 2H), 6.95 (s, 1H), 6.58 (d, J = 9.5 Hz, 1H), 6.35 (tt, J = 1.3, 52.0 Hz, 1H), 4.45 (dt, J = 3.9, 14.8 Hz, 2H), 4.20-4.07(m, 1H), 3.24- 3.21 (m, 2H), 3.08-3.03 (m, 2H), 2.73-2.65 (m, 2H), 1.98-1.77 (m, 3H), 1.58- 1.42 (m, 1H) |
| 521 | 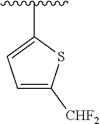 [SMILES: *c1ccc(C(F)F)s1] | 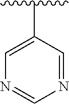 [SMILES: *c1cncnc1] | (R)-N-(1-(4-amino- 7-(pyrimidin-5- yl)pyrrolo[2,1- f][1,2,4]triazin-5- yl)piperidin-3-yl)- 5- (difluoromethyl) thiophene-2- carboxamide | 471.1 | E: 1.336 F: 1.127 | (400 MHz, DMSO-d₆) δ ppm 9.48 (s, 2H), 9.09 (s, 1H), 8.49 (d, J = 7.8 Hz, 1H), 8.23-8.04 (m, 1H), 7.92 (s, 1H), 7.73 (d, J = 4.2 Hz, 1H), 7.31 (s, 1H), 7.19 (d, J = 3.9 Hz, 1H), 6.97 (br s, 1H), 6.47 (d, J = 9.5 Hz, 1H), 4.19-4.10 (m, 1H), 3.25-3.21 (m, 1H), 3.10-3.02 (m, 1H), 2.79-2.64 (m, 2H), 2.01- 1.74(m, 3H), 1.60-1.43 (m, 1H) |
| 522 | 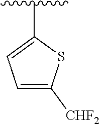 [SMILES: *c1ccc(C(F)F)s1] | 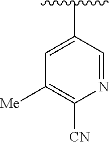 [SMILES: *c1cnc(C#N)c(C)c1] | ((R)-N-(1-(4- amino-7-(6-cyano- 5-methylpyridin-3- yl)pyrrolo[2,1- f][1,2,4]triazin-5- yl)piperidin-3-yl)- 5- (difluoromethyl) thiophene-2- carboxamide | 509.1 | E: 1.745 F: 1.448 | (400 MHz, DMSO-d₆) δ ppm 9.31 (d, J = 2.0 Hz, 1H), 8.67 (d, J = 1.3 Hz, 1H), 8.59 (d, J = 7.8 Hz, 1H), 8.22- 8.07 (m, 1H), 7.96 (s, 1H), 7.87-7.77 (m, 2H), 7.58- 7.13 (m, 5H), 7.09-6.90 (m, 1H), 4.18 (br d, J = 7.0 Hz, 1H), 3.24 (br s, 1H), 3.12- 3.02 (m, 1H), 2.79-2.69 (m, 2H), 2.00-1.72 (m, 3H), 1.62-1.46 (m, 1H) |
| 523 | 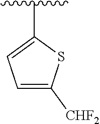 [SMILES: *c1ccc(C(F)F)s1] | 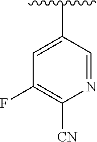 [SMILES: *c1cnc(C#N)c(F)c1] | (R)-N-(1-(4-amino- 7-(6-cyano-5- fluoropyridin-3- yl)pyrrolo[2,1- f][1,2,4]triazin-5- yl)piperidin-3-yl)- 5- (difluoromethyl) thiophene-2- carboxamide | 513.0 | E: 1.862 | (400 MHz, DMSO-d₆) δ ppm 9.37 (t, J = 1.7 Hz, 1H), 8.85 (d, J = 12.7 Hz, 1H), 8.60 (d, J = 9.5 Hz, 1H), 8.02 (s, 1H), 7.83 (d, J = 3.7 Hz, 1H), 7.54 (s, 1H), 7.48 (s, 1H), 7.32 (t, J = 56.0 Hz, 1H), 6.52 (s, 2H), 4.18 (br d. J = 7.0 Hz, 1H), 3.24 (br s, 1H), 3.12-3.02 (m, 1H), 2.79-2.69 (m, 2H), 2.00-1.72 (m, 3H), 1.62-1.46 (m, 1H) |
| 524 | 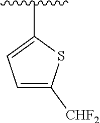 [SMILES: *c1ccc(C(F)F)s1] | 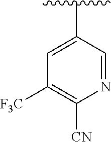 [SMILES: *c1cnc(C#N)c(C(F)(F)F)c1] | (R)-N-(1-(4-amino- 7-(6-cyano-5- (trifluoromethyl)py ridin-3- yl)pyrrolo[2,1- f][1,2,4]triazin-5- yl)piperidin-3-yl)- 5- (difluoromethyl) thiophene-2- carboxamide. | 563.1 | E: 2.032 F: 1.746 | (400 MHz, DMSO-d₆) δ ppm 9.79-9.66 (m, 1H), 9.28- 9.12 (m, 1H), 8.68-8.48 (m, 1H), 8.41-8.21 (m, 1H), 8.12-7.90 (m, 1H), 7.85-7.79 (m, 1H), 7.68-7.61 (m, 1H), 7.50-7.10 (m, 2H), 4.30- 4.07 (m, 1H), 3,25-3.01 (m, 2H), 2.85-2.63 (m, 2H), 2.09-1.74 (m, 2H), 1.78- 1.40 (m, 2H), 1.34-1.12 (m, 1H) |
| 525 | 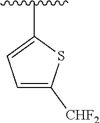 [SMILES: *c1ccc(C(F)F)s1] | 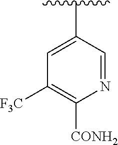 [SMILES: *c1cnc(C(N)=O)c(C(F)(F)F)c1] | R)-5-(4-amino-5- (3-(5- (difluoromethyl) thiophene-2- carboxamido) piperidin-1- yl)pyrrolo[2,1- f][1,2,4]triazin-7- yl)-3- (trifluoromethyl) picolinamide. | 581.0 | E: 1.514 F: 1.263 | (400 MHz, DMSO-d₆) δ ppm 9.51 (d, J = 1.8 Hz, 1H), 9.01 (d, J = 1.8 Hz, 1H), 8.60 (d, J = 7.8 Hz, 1H), 8.09 (s, 2H), 7.97 (s, 1H), 7.86-7.81 (m, 1H), 7.75 (s, 1H), 7.53-7.15 (m, 4H), 7.10-6.84 (m, 1H), 4.22-4.14 (m, 1H), 3.25-3.19 (m, 1H), 3.11- 3.02 (m, 1H), 2.75 (br s, 2H), 2.01-1.78 (m, 2H), 1.56 (br d. J = 9.8 Hz, 1H) |
| 526 | 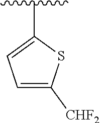 [SMILES: *c1ccc(C(F)F)s1] | 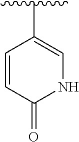 [SMILES: *c1ccc(=O)[nH]c1] | (R)-N-(1-(4-amino- 7-(6-oxo-1,6- dihydropyridin-3- yl)pyrrolo[2,1- f][1,2,4]triazin-5- yl)piperidin-3-yl)- 5- (difluoromethyl) thiophene-2- carboxamide. | 486.1 | E: 1.299 F: 1.089 | |
| 527 | 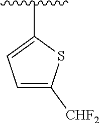 [SMILES: *c1ccc(C(F)F)s1] | 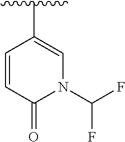 [SMILES: *c1ccc(=O)n(C(F)F)c1] | (R)-N-(1-(4-amino- 7-(1- (difluoromethyl)-6- oxo-1,6- dihydropyridin-3- yl)pyrrolo[2,1- f][1,2,4]triazin-5- yl)piperidin-3-yl)- 5- (difluoromethyl) thiophene-2- carboxamide. | 536.1 | E: 1.702 F: 1.393 | (400 MHz, DMSO-d₆) δ ppm 8.74 (d, J = 2.4 Hz, 1H), 8.62-8.51 (m, 1H), 8.19 (dd, J = 2.4, 9.8 Hz, 1H), 8.00- 7.77 (m, 3H), 7.51-7.47 (m, 2H), 7.47-7.12 (m, 2H), 6.84 (s, 1H), 4.16 (br dd, J = 2.8, 4.6 Hz, 2H), 3,18- 3.00 (m, 2H), 2.76-2.68 (m, 2H), 1.97-1.77 (m, 3H), 1.59-1.43 (m, 1H) |
| 528 | 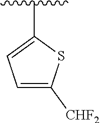 [SMILES: *c1ccc(C(F)F)s1] | 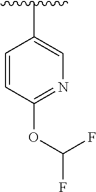 [SMILES: *c1ccc(OC(F)F)nc1] | (R)-N-(1-(4-amino- 7-(6- (difluoromethoxy) pyridin-3- yl)pyrrolo[2,1- f][1,2,4]triazin-5- yl)piperidin-3-yl)- 5- (difluoromethyl) thiophene-2- carboxamide. | 536.1 | E: 2.026 F: 1.694 | (400 MHz, DMSO-d₆) δ ppm 8.90 (d, J = 2.3 Hz, 1H), 8.62-8.49 (m, 2H), 7.94 (s, 1H), 7.85-7.79 (m, 2H), 7.48 (td, J = 1.7, 3.8 Hz, 1H), 7.34-7.15 (m, 4H), 4.23- 4.16 (m, 1H), 3.28-3.23 (m, 1H), 3.12-3.04 (m, 2H), 2.79-2.68 (m, 2H), 2.00-1.81 (m, 3H), 1.52 (br s, 1H) |
| 529 | 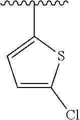 [SMILES: *c1ccc(Cl)s1] | 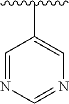 [SMILES: *c1cncnc1] | (R)-N-(1-(4-amino- 7-(pyrimidin-5- yl)pyrrolo[2,1- f][1,2,4]triazin-5- yl)piperidin-3-yl)- 5-chlorothiophene- 2-carboxamide | 455.0 | E: 1.532 | (400 MHz, DMSO-d₆) δ ppm 9.48 (s, 2H), 9.09 (s, 1H). 8.49 (d, J = 7.8 Hz, 1H), 8.23- 8.04 (m, 1H), 7.92 (s, 1H), 7.73 (d, J = 4.2 Hz, 1H), 7.31 (s, 1H), 7.19 (d, J = 3.9 Hz, 1H), 6.97 (br s, 1H), 4.19-4.10 (m, 1H), 3.25- 3.21 (m, 1H), 3.10-3.02 (m, 1H), 2.79-2.64 (m, 2H), 2.01-1.74(m, 3H), 1.60-1.43 (m, 1H) |
| 530 | 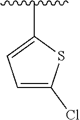 [SMILES: *c1ccc(Cl)s1] | 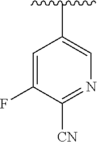 [SMILES: *c1cnc(C#N)c(F)c1] | (R)-N-(1-(4-amino- 7-(6-cyano-5- fluoropyridin-3- yl)pyrrolo[2,1- f][1,2,4]triazin-5- yl)piperidin-3-yl)- 5-chlorothiophene- 2-carboxamide | 497.1 | F: 1.592 | (400 MHz, DMSO-d₆) δ ppm 9.37 (t, J = 1.5 Hz, 1H), 8.85 (dd, J = 1.6, 11.4 Hz, 1H), 8.50 (d, J = 7.8 Hz, 1H), 8.25 (br d, J = 19.6 Hz, 1H),8.01 (s, 1H), 7.73 (d, J = 4.2 Hz, 1H), 7.53 (s, 1H), 7.19 (d, J = 3.9 Hz, 1H), 7.06-6.98 (m, 1H), 4.19-4.10 (m, 1H), 3.25-3.21 (m, 1H), 3.10-3.02 (m, 1H), 2.79- 2.64 (m, 2H), 2.01- 1.74(m, 3H), 1.60-1.43 (m, 1H) |
| 531 | 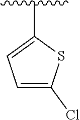 [SMILES: *c1ccc(Cl)s1] | 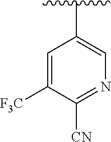 [SMILES: *c1cnc(C#N)c(C(F)(F)F)c1] | (R)-N-(1-(4-amino- 7-(6-cyano-5- (trifluoromethyl)py ridin-3- yl)pyrrolo[2,1- f][1,2,4]triazin-5- yl)piperidin-3-yl)- 5-chlorothiophene- 2-carboxamide | 547.1 | E: 2.081 F: 1.780 | (400 MHz, DMSO-d₆) δ ppm 8.59 (d, J = 7.0 Hz, 1H), 8.13 (s, 1H), 7.99 (d, J = 0.8 Hz, 1H), 7.76 (d, J = 2.5 Hz, 1H), 7.60 (dd, J = 2.5, 8.5 Hz, 1H), 7.35 (s, 1H), 7.28 (d, J = 8.8 Hz, 1H), 6.87- 6.66 (m, 2H), 4.19-4.10 (m, 1H), 3.25-3.21 (m, 1H), 3.10-3.02 (m, 1H), 2.79- 2.64 (m, 2H), 2.01-1.74(m, 3H), 1.60-1.43 (m, 1H) |
| 532 | 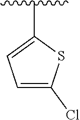 [SMILES: *c1ccc(Cl)s1] | 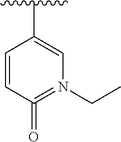 | (R)-N-(1-(4-amino- 7-(1-ethyl-6-oxo- 1,6-dihydropyridin- 3-yl)pyrrolo[2,1- f][1,2,4]triazin-5- yl)piperidin-3-yl)- 5-chlorothiophene- 2-carboxamide | 499.1 | E: 1.483 F: 1.231 | (400 MHz, DMSO-d₆) δ ppm 8.53-8.40 (m, 2H), 8.05 (dd, J = 2.6, 9.7 Hz, 1H), 7.94 (s, 1H), 7.72 (d, J = 3.9 Hz, 1H), 7.19 (d, J = 3.9 Hz, 1H), 7.00 (s, 1H), 6.50 (d, J = 9.8 Hz, 1H), 4.22-4.13 (m, 1H), 3.99 (q, J = 7.1 Hz, 4H), 3.27 (br s, 1H), 3.11- 3.06 (m, 1H), 2.76-2.67 (m, 2H),2.00-1.78 (m, 3H), 1.60-1.48 (m, 1H), 1.27 (t, J = 7.1 Hz, 3H) |
| 533 | 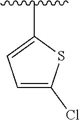 [SMILES: *c1ccc(Cl)s1] | 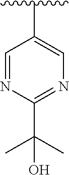 [SMILES: *c1cnc(C(C)(C)O)nc1] | (R)-N-(1-(4-amino- 7-(2-(2- hydroxypropan-2- yl)pyrimidin-5- yl)pyrrolo[2,1- f][1,2,4]triazin-5- yl)piperidin-3-yl)- 5-chlorothiophene- 2-carboxamide | 514.2 | E: 1.684 F: 1.329 | (400 MHz, DMSO-d₆) δ ppm 9.40 (s, 2H), 8.47 (d, J = 7.5 Hz, 1H), 8.07 (br s, 1H), 7.89 (s, 1H), 7.72 (d, J = 4.0 Hz, 1H), 7.23 (s, 1H), 7.18 (d, J = 4.0 Hz, 1H), 7.07- 6.83 (m, 1H), 5.21-4.92 (m, 1H), 4.21-4.08 (m, 1H), 3.25-3.18 (m, 1H), 3.11-3.02 (m, 1H), 2.78-2.67 (m, 2H), 2.00-1.76 (m, 3H), 1.52 (s, 7H). |
| 534 | 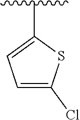 [SMILES: *c1ccc(Cl)s1] | 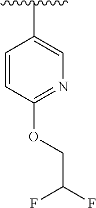 [SMILES: *c1ccc(OCC(F)F)nc1] | (R)-N-(1-(4-amino- 7-(6-(2,2- difluoroethoxy)pyri din-3- yl)pyrrolo[2,1- f][1,2,4]triazin-5- yl)piperidin-3-yl)- 5-chlorothiophene- 2-carboxamide | 534.1 | E: 2.084 F: 1.691 | (400 MHz, DMSO-d₆) δ ppm 8.84 (d, J = 2.7 Hz, 1H), 8.49 (d, J = 7.8 Hz, 1H), 8.41 (dd, J = 2.2, 8.8 Hz, 1H), 7.94 (s, 1H), 7.72 (d, .) = 4.2 Hz, 1H), 7.67-7.55 (m, 2H), 7.19 (d, J = 3.9 Hz, 1H), 7.13 (s, 1H), 7.06 (d, J = 8.8 Hz, 1H), 6.59-6.24 (m, 1H), 4.63 (dt, J = 3.2, 14.9 Hz, 2H),4.20-4.13 (m, 1H), 3.27-3.23 (m, 1H), 3.12-3.06 (m, 1H), 2.79-2.68 (m, 2H), 1.99-1.77 (m, 3H), 1.60-1.45 (m, 1H) |
| 535 | 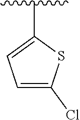 [SMILES: *c1ccc(Cl)s1] | 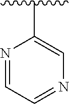 [SMILES: *c1cnccn1] | (R)-N-(1-(4-amino- 7-(pyrazin-2- yl)pyrrolo[2,1- f][1,2,4]triazin-5- yl)piperidin-3-yl)- 5-chlorothiophene- 2-carboxamide | 455.1 | E: 1.629 F: 1.337 | (400 MHz, DMSO-d₆) δ ppm 8.49 (d, J = 2.5 Hz, 1H), 8.46 (d, J = 8.0 Hz, 1H), 8.11 (dd, J = 2.6, 9.6 Hz, 1H), 7.90 (s, 1H), 7.71 (d, J = 4.0 Hz, 1H), 7.18 (d, J = 4.3 Hz, 2H), 6.95 (s, 1H), 6.58 (d, J = 9.5 Hz, 1H), 6.35 (tt, J = 1.3, 52.0 Hz, 1H), 4.45 (dt, J = 3.9, 14.8 Hz, 2H), 4.20-4.07(m, 1H), 3.24- 3.21 (m, 1H), 3.08-3.03 (m, 1H), 2.73-2.65 (m, 3H), 1.98-1.77 (m, 3H), 1.58- 1.42 (m, 1H) |
| 536 | 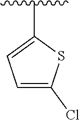 [SMILES: *c1ccc(Cl)s1] | 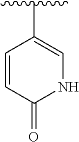 [SMILES: *c1ccc(=O)[nH]c1] | (R)-N-(1-(4-amino- 7-(6-oxo-1,6- dihydropyridin-3- yl)pyrrolo[2,1- f][1,2,4]triazin-5- yl)piperidin-3-yl)- 5-chlorothiophene- 2-carboxamide | 470.1 | E: 1.371 | (400 MHz, DMSO-d₆) δ ppm 11.90 (s, 1H), 8.47 (d, J = 7.3 Hz, 1H), 8.33 (d, J = 2.2 Hz, 1H), 8.02 (dd, J = 2.7, 9.8 Hz, 1H), 7.92 (s, 1H), 7.71 (d, J = 3.9 Hz, 1H), 7.19 (d, J = 3.9 Hz, 2H), 7.01 (s, 1H), 6.45 (d, J = 9.5 Hz, 1H), 4.15 (br dd, J = 2.6, 7.7 Hz, 1H), 3.23 (br s, 1H), 3.05 (br d, J = 3.7 Hz, 1H), 2.75-2.68 (m, 2H), 1.87 (br d, J = 7.3 Hz, 4H), 1.57-1.47 (m, 1H) |
| 537 | 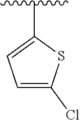 [SMILES: *c1ccc(Cl)s1] | 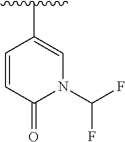 [SMILES: *c1ccc(=O)n(C(F)F)c1] | (R)-N-(1-(4-amino- 7-(1- (difluoromethyl)-6- oxo-1,6- dihydropyridin-3- yl)pyrrolo[2,1- f][1,2,4]triazin-5- yl)piperidin-3-yl)- 5-chlorothiophene- 2-carboxamide. | 520.1 | E: 1.767 F: 1.430 | (400 MHz, DMSO-d₆) δ ppm 8.78 (d, J = 2.3 Hz, 1H), 8.46 (d, J = 7.5 Hz, 1H), 8.19 (dd, J = 2.6, 9.9 Hz, 1H), 7.90 (s, 1H), 7.91 (t, J = 75.3 Hz, 1H), 7.71 (d, J = 4.0 Hz, 1H), 7.18 (d, J = 4.0 Hz, 1H), 7.11 (s, 1H), 7.00- 6.80 (m, 1H), 6.65 (d, J = 9.8 Hz, 1H), 4.10 (br s, 1H), 3.22-3.17 (m, 1H), 3.07-3.01 (m, 1H), 2.74-2.66 (m, 2H), 1.98-1.74 (m, 4H), 1.58- 1.38 (m, 1H) |
| 538 | 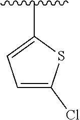 [SMILES: *c1ccc(Cl)s1] | 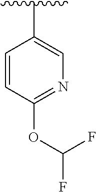 [SMILES: *c1ccc(OC(F)F)nc1] | (R)-N-(1-(4-amino- 7-(6- (difluoromethoxy) pyridin-3- yl)pyrrolo[2,1- f][1,2,4]triazin-5- yl)piperidin-3-yl)- 5-chlorothiophene- 2-carboxamide | 520.1 | E: 2.142 F: 1.740 | (400 MHz, DMSO-d₆) δ ppm 8.90 (d, J = 2.4 Hz, 1H), 8.56 (dd, J = 2.4, 8.8 Hz, 1H), 8.48 (d, J = 7.8 Hz, 1H), 7.98-7.54 (m, 3H), 7.25 -7.16 (m, 3H), 6.65 (d, J = 9.8 Hz, 1H), 4.22-4.15 (m, 1H), 3.24 (brd, J = 2.9 Hz, 1H), 3.07 (br s, 1H), 2.78-2.69 (m, 2H), 1.97-1.78 (m, 4H), 1.52 (br s, 1H) |
| 539 | 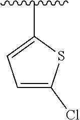 [SMILES: *c1ccc(Cl)s1] | 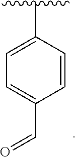 [SMILES: *c1ccc(C=O)cc1] | (R)-N-(1-(4-amino- 7-14- formylphenyl)pyrrolo [2,1- f][1,2,4]triazin-5- vl)piperidin-3-yl)-5- chlorothiophene-2- carboxamide | 481.1 | E: 1.914 F: 1.535 | (400 MHz, DMSO-d6) δ ppm 10.00 (s, 1H), 8.54-8.31 (m, 3H), 8.14-7.86 (m, 4H), 7.81-7.63 (m, 2H), 7.31- 6.95 (m, 2H), 4.24-4.09 (m, 1H), 3.23 (br dd, J = 2.0, 10.8 Hz, 1H), 3.13-3.03 (m, 1H), 2.84-2.65 (m, 2H), 2.00-1.76 (m, 3H), 1.62-1.40 (m, 1H) |
| 540 | 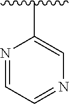 [SMILES: *c1cnccn1] | 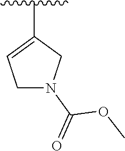 [SMILES: *C1=CCN(C(=O)OC)C1] | (R)-3-(4-amino-5- (3-(pyrazine-2- carboxamido) piperidin-1- yl)pyrrolo[2,1- f][ 1,2,4]triazin-7- yl)-2,5-dihydro- 1H-pyrrole-1- carboxylate | 464.2 | E: 1.416 F: 1.092 | (400 MHz, DMSO-d₆) δ ppm 9.17 (d, J = 1.5 Hz, 1H), 8.88 (d, J = 2.3 Hz, 1H), 8.81- 8.70 (m, 2H), 7.88 (s, 1H), 6.78-6.70 (m, 2H), 4.58- 4.49 (m, 2H), 4.31-4.21 (m, 3H) 3.65 (d, J = 3.8 Hz, 3H), 3.17-3.13 (m, 2H), 3.03-2.92 (m, 2H),2.84-2.68 (m, 2H), 1.93-1.76 (m, 3H), 1.71-1.60 (m, 1H). |

### Example 541

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(2-(difluoromethyl)pyridin-4-yl)thiophene-2-carboxamide

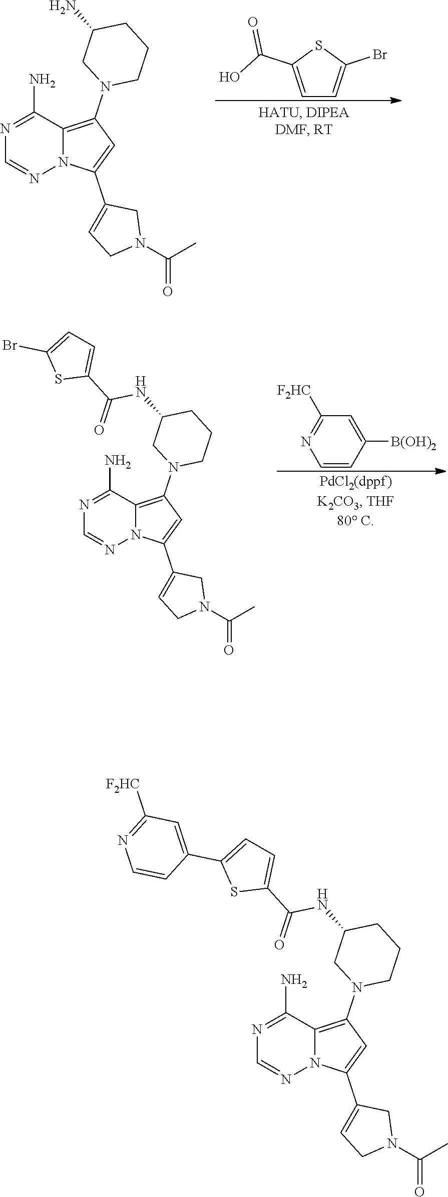 [SMILES: CC(=O)N1CC=C(c2cc(N3CCC[C@@H](N)C3)c3c(N)ncnn23)C1 ; O=C(O)c1ccc(Br)s1 ; CC(=O)N1CC=C(c2cc(N3CCC[C@@H](NC(=O)c4ccc(Br)s4)C3)c3c(N)ncnn23)C1 ; OB(O)c1ccnc(C(F)F)c1 ; CC(=O)N1CC=C(c2cc(N3CCC[C@@H](NC(=O)c4ccc(-c5ccnc(C(F)F)c5)s4)C3)c3c(N)ncnn23)C1]

### Example 541A. (R)—N-(1-(7-(1-acetyl-2,5-dihydro-1H-pyrrol-3-yl)-4-aminopyrrolo[2,1-f][1,2,4]triazin-5-yl)piperidin-3-yl)-5-bromothiophene-2-carboxamide

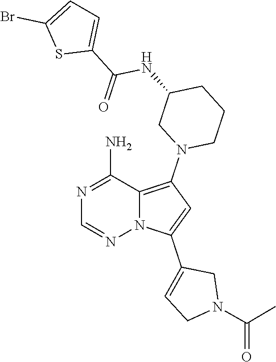 [SMILES: CC(=O)N1CC=C(c2cc(N3CCC[C@@H](NC(=O)c4ccc(Br)s4)C3)c3c(N)ncnn23)C1]

Intermediate 32 (100 mg, 0.293 mmol), 5-bromothiophene-2-carboxylic acid (60.6 mg, 0.293 mmol), BOP (194 mg, 0.439 mmol) and DIEA (0.153 mL, 0.879 mmol) were dissolved in DMF (4 mL). The mixture was stirred at 25° C. for 1 h, then was partitioned between ethyl acetate and water (50 mL). The organic layer was dried over Na₂SO₄ and concentrated. Residue was purified by flash chromatography to afforded Example 541A as an off-white solid (150 mg, 97% yield). LC-MS Method E: RT=1.30 min, [M]⁺ & [M+2H]⁺=530.3 & 532.3.

### Example 541

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-(2-(difluoromethyl)pyridin-4-yl)thiophene-2-carboxamide

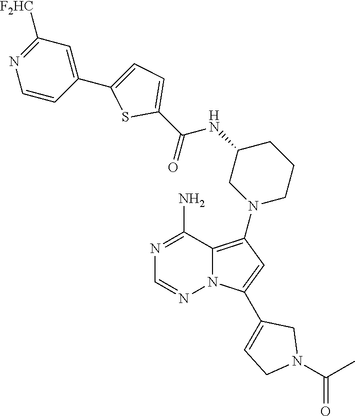 [SMILES: CC(=O)N1CC=C(c2cc(N3CCC[C@@H](NC(=O)c4ccc(-c5ccnc(C(F)F)c5)s4)C3)c3c(N)ncnn23)C1]

A mixture of Example 541A (40 mg, 0.075 mmol), (2-(difluoromethyl)pyridin-4-yl)boronic acid (15.7 mg, 0.090 mmol), PdCl₂(dppf) (2.8 mg, 3.8 μmol) and K₂CO₃ (31.3 mg, 0.226 mmol) in dioxane (2 mL)-water (1 mL), was stirred at 80° C. for 18 h. The mixture was partitioned between ethyl acetate and water. The organic phase was dried over Na₂SO₄ and concentrated. The residue was purified by flash chromatography which afforded Example 541 (5.6 mg, 12% yield) as an off-white solid. MS: [M+H]⁺=579.2; ¹H NMR (400 MHz, DMSO-d₆) δ ppm 8.72 (d, J=4.9 Hz, 1H), 8.54 (br d, J=6.8 Hz, 1H), 8.03-7.81 (m, 9H), 7.15-6.85 (m, 3H), 6.82-6.66 (m, 2H), 4.73-4.66 (m, 1H), 4.54-4.42 (m, 2H), 4.27 (br s, 1H), 4.22-4.07 (m, 2H), 3.91 (s, 1H), 3.18 (s, 1H), 3.03 (br dd, J=2.4, 8.3 Hz, 1H), 2.78-2.65 (m, 3H), 2.03 (d, J=16.1 Hz, 3H), 1.98-1.78 (m, 5H), 1.58-1.46 (m, 1H), 1.27-1.19 (m, 2H).

The following examples in Table 39 were prepared using the same procedures as described in Example 543. In step 1, Intermediate 32 and 33 were coupled with 5-bromothiophene-2-carboxylic acid through acid-amine coupling. In step-2, products obtained from step 1 were treated with appropriate boronic acid or boronate ester to get Examples 542-554 through Suzuki coupling. Examples 553 and 554 were synthesized from Examples 545 and 546 respectively by treating with 4N HCl in dioxane.

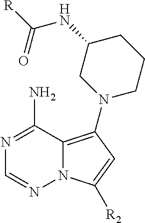

TABLE 39

| | | | | | HPLC | |
|---|---|---|---|---|---|---|
| Ex | | | | LCMS | Method, | |
| No. | R | R₂ | Name | (M + H)⁺ | RT (min.) | ¹H NMR |
| 542 | 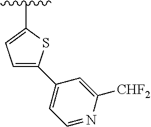 [SMILES: *c1ccc(-c2ccnc(C(F)F)c2)s1] | 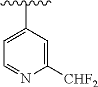 [SMILES: *c1ccnc(C(F)F)c1] | (R)-N-(1-(4- amino-7-(2- (difluoromethyl) pyridin-4-yl) pyrrolo[2,1-f] [1,2,4]triazin-5- yl)piperidin-3- yl)-5-(2- | 597.1 | E: 1.688 F: 1.433 | (400 MHz, DMSO-d₆) δ ppm 8.71 (dd, J = 5.3, 11.1 Hz, 2H), 8.56 (d, J = 7.3 Hz, 1H), 8.50 (s, 1H), 8.32 (br d, J = 5.4 Hz. 1H), 8.18 (br s, 1H), 8.01-7.91 (m, 4H), 7.88 (br d, J = 5.1 Hz, 1H), 7.47 (s, 1H), 6.98 (dt, J = |
| | | | (difluoromethyl) | | | 11.0, 53.3 Hz, 3H), 4.25- |
| | | | pyridin-4-yl) | | | 4.13 (m, 1H), 3.28-3.24 (m, |
| | | | thiophene- | | | 1H), 3.13-3.02 (m, 1H), |
| | | | 2-carboxamide | | | 2.84-2.69 (m, 2H), 2.01- |
| | | | | | | 1.78 (m, 3H), 1.65-1.44 |
| | | | | | | (m, 1H) |
| 543 | 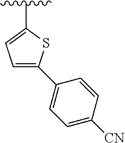 [SMILES: *c1ccc(-c2ccc(C#N)cc2)s1] | 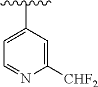 [SMILES: *c1ccnc(C(F)F)c1] | (R)-N-(1-(4- amino-7-(2- (difluoromethyl) pyridin-4-yl) pyrrolo[2,1-f] [1,2,4]triazin-5- yl)piperidin-3- yl)-5-(4-cyano- phenyl) | 571.2 | E: 1.855 | |
| | | | thiophene- | | | |
| | | | 2-carboxamide | | | |
| 544 | 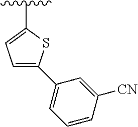 [SMILES: *c1ccc(-c2cccc(C#N)c2)s1] | 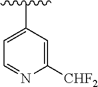 [SMILES: *c1ccnc(C(F)F)c1] | (R)-N-(1-(4- amino-7-(2- (difluoromethyl) pyridin-4-yl) pyrrolo[2,1-f] [1,2,4]triazin- 5-yl)piperidin- 3-yl)-5-(3- | 571.1 | E: 1.854 F: 1.567 | (400 MHz, DMSO-d₆) δ ppm 8.71 (d, J = 5.6 Hz, 1H), 8.54- 8.40 (m, 3H), 8.31 (d, J = 5.1 Hz, 1H), 8.23 (t, J = 1.5 Hz, 1H), 8.07-7.99 (m, 2H), 7.87 (d, J = 3.9 Hz, 1H), 7.82 (d, J = 7.8 Hz, 1H), 7.72 (d, J = 3.9 Hz, 1H), 7.68- |
| | | | cyanophenyl) | | | 7.62 (m, 1H), 7.49 (s, 1H), |
| | | | thiophene-2- | | | 7.25 (br s, 1H), 7.13-6.81 |
| | | | carboxamide | | | (m, 1H), 4.26-4.17 (m, 1H), |
| | | | | | | 3.31-3.26 (m, 1H), 3.09 (br s, |
| | | | | | | 1H), 2.84-2.71 (m, 2H), |
| | | | | | | 2.02-1.81 (m, 3H), 1.64- |
| | | | | | | 1.49 (m, 1H) |
| 545 | 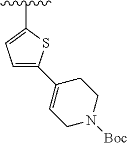 [SMILES: *c1ccc(C2=CCN(C(=O)OC(C)(C)C)CC2)s1] | 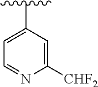 [SMILES: *c1ccnc(C(F)F)c1] | tert-butyl (R)-4-(5- ((1-(4-amino-7-(2- (difluoromethyl) pyridin-4-yl) pyrrolo[2,1-f] [1,2,4]triazin-5- yl)piperidin-3- yl)carbamoyl) thiophen-2-yl)- | 651.3 | E: 2.106 F: 1.817 | (400 MHz, DMSO-d₆) δ 8.69 (d, J = 5.1 Hz, 1H), 8.50 (s, 1H), 8.37 (d, J = 8.1 Hz, 1H), 8.33-8.29 (m, 1H), 8.22-8.10 (m, 1H), 7.99 (s, 1H), 7.74 (d, J = 3.9 Hz, 1H), 7.46 (s, 1H), 7.12-6.78 (m, 3H), 6.37 (br s, 1H), 4.19 (br d, J = 1.5 Hz, 3H), 3.46 (br t, J = |
| | | | 3,6-dihydro- | | | 5.5 Hz, 2H), 3.28-3.23 (m, |
| | | | pyridine-1(2H)- | | | 1H), 3.12-3.02 (m, 1H), 2.81- |
| | | | carboxylate | | | 2.67 (m, 2H), 2.29-2.16 (m, |
| | | | | | | 2H), 1.99-1.74 (m, 3H), 1.61- |
| | | | | | | 1.51 (m, 1H), 1.43 (s, 9H) |
| 546 | 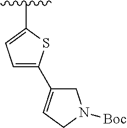 [SMILES: *c1ccc(C2=CCN(C(=O)OC(C)(C)C)C2)s1] | 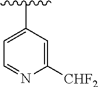 [SMILES: *c1ccnc(C(F)F)c1] | tert-butyl (R)-3-(5- ((1-(4-amino-7-(2- (difluoromethyl) pyridin-4-yl) pyrrolo[2,1-f] [1,2,4]triazin-5- yl)piperidin-3- yl)carbamoyl) thiophen-2-yl)- 2,5-dihydro- 1H-pyrrole-1- carboxylate | 637.3 | E: 2.019 F: 1.742 | |
| 548 | 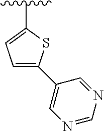 [SMILES: *c1ccc(-c2cncnc2)s1] | 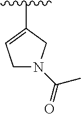 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 5-(pyrimidin-5- yl)thiophene-2- carboxamide | 530.2 | E: 1.122 F: 0.946 | (400 MHz, DMSO-d6) δ ppm 9.20-9.11 (m, 3H), 8.51 (d, J = 7.5 Hz, 1H), 8.33 (br s, 1H), 7.94 (s, 1H), 7.90 (d, J = 4.0 Hz, 1H), 7.78 (d, J = 4.0 Hz, 1H), 7.32-7.00 (m, 1H), 6.86-6.72 (m, 2H), 4.70 (br d, J = 1.5 Hz, 1H), 4.54-4.43 (m, 2H), 4.28 (br d, J = 2.5 Hz, |
| | | | | | | 1H), 4.19 (br s, 1H), 3.24- |
| | | | | | | 3.21 (m, 1H), 3.08-3.03 (m, |
| | | | | | | 1H), 2.75-2.68 (m, 1H), 2.03 |
| | | | | | | (d, J = 15.8 Hz, 3H), 1.87 (br d, |
| | | | | | | J = 3.0 Hz, 3H), 1.56 (br s, 1H) |
| 549 | 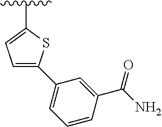 [SMILES: *c1ccc(-c2cccc(C(N)=O)c2)s1] | 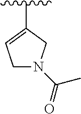 [SMILES: *C1=CCN(C(C)=O)C1] | (R)-N-(1-(7-(1- acetyl-2,5~dihydro- 1H-pyrrol-3-yl)-4- aminopyrrolo[2,1- f][1,2,4]triazin-5- yl)piperidin-3- yl)-5-(3- carbamoylphenyl) | 571.2 | E: 1.183 F: 1.002 | (400 MHz, DMSO-d6) δ ppm 8.42 (d, J = 7.8 Hz, 1H), 8.19 (s, 1H), 8.12 (br s, 2H), 7.92- 7.77 (m, 4H), 7.62 (dd, J = 1.0, 4.0 Hz, 1H), 7.53 (t, J = 7.8 Hz, 1H), 7.47 (br s, 1H), 7.01 (br s, 1H), 6.83- 6.69 (m, 2H), 4.70 (br s, 1H), |
| | | | thiophene-2- | | | 4.49 (br d, J = 11.0 Hz, 2H), |
| | | | carboxamide | | | 4.27 (br d, J = 2.0 Hz, 1H), |
| | | | | | | 4.19-4.09 (m, 1H), 3.23-3.19 |
| | | | | | | (m, 1H), 3.07-2.98 (m, 1H), |
| | | | | | | 2.74-2.65 (m, 2H), 2.03 (d, J = |
| | | | | | | 16.5 Hz, 3H), 1.97-1.78 (m, |
| | | | | | | 3H), 1.55 (br s, 1H) |
| 550 | 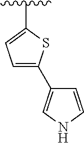 [SMILES: *c1ccc(-c2cc[nH]c2)s1] | 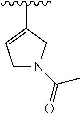 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 5-(1H-pyrazol-4- yl)thiophene-2- carboxamide | 518.2 | E: 1.229 F: 0.965 | (400 MHz, DMSO-d6) δ ppm 13.10 (s, 1H), 8.29 (br d, J = 7.6 Hz, 1H), 8.18-7.81 (m, 4H), 7.74 (br d, J = 3.4 Hz, 1H), 7.22 (br d, J = 3.2 Hz, 1H), 7.06-6.59 (m, 3H), 4.76-4.65 (m, 1H), 4.49 (br d, J = 11.2 Hz, 2H), 4.27 (br dd, J = 1.0, 2.0 Hz, 1H), 4.20- 4.01 (m, 1H), 3.20 (br dd, J = 4.5, |
| | | | | | | 5.5 Hz, 1H), 3.05-2.98 (m, 1H), |
| | | | | | | 2.75-2.62 (m, 2H), 2.03 (br d, J = |
| | | | | | | 16.4 Hz, 3H), 1.96-1.73 (m, 3H), |
| | | | | | | 1.60-1.37 (m, 1H) |
| 551 | 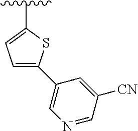 [SMILES: *c1ccc(-c2cncc(C#N)c2)s1] | 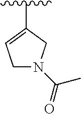 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)-5- (5-cyanopyridin-3- yl)thiophene-2- carboxamide | 554.2 | E: 1.439 F: 1.147 | (400 MHz, DMSO-d₆) δ ppm 9.21 (d, J = 2.2 Hz, 1H), 8.98 (d, J = 1.5 Hz, 1H), 8.70 (s, 1H), 8.51 (d, J = 7.6 Hz, 1H), 8.17-7.95 (m, 1H), 7.93-7.87 (m, 2H), 7.81 (d, J = 3.9 Hz, 1H), 6.93 (br s, 1H), 6.81-6.70 (m, 2H), 4.69 (br s, 1H), 4.49 (br d, J = 11.0 Hz, 2H), 4.30- |
| | | | | | | 4.24 (m, 1H), 4.16 (br dd, J = |
| | | | | | | 1.5, 3.9 Hz, 1H), 3.21 (br d, |
| | | | | | | J = 4.2 Hz, 1H), 3.06-2.96 (m, |
| | | | | | | 1H), 2.76-2.65 (m, 2H), 2.03 |
| | | | | | | (d, J = 16.1 Hz, 3H), 1.98-1.77 |
| | | | | | | (m, 3H), 1.60-1.49 (m, 1H) |
| 552 | 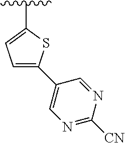 [SMILES: *c1ccc(-c2cnc(C#N)nc2)s1] | 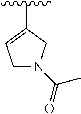 [SMILES: *C1=CCN(C(C)=O)C1] | (R)-N-(1-(7-(1- acetyl-2,5-dihydro- 1H-pyrrol-3-yl)-4- aminopyrrolo[2,1- f][1,2,4]triazin-5- yl)piperidin-3-yl)- 5-(2-cyano- pyrimidin-5- yl)thiophene-2- | 555.2 | E: 1.502 F: 1.234 | (400 MHz, DMSO-d₆) δ ppm 9.38 (s, 2H), 8.56 (d, J = 7.8 Hz, 1H), 7.99-7.92 (m, 2H), 7.90 (s, 1H), 7.58 (br d, J = 7.3 Hz, 1H), 6.81-6.70 (m, 2H), 4.69 (br d, J = 1.0 Hz, 1H), 4.54-4.42 (m, 2H), 4.26 (br d, J = 2.8 Hz, 1H), 4.22- 4.12 (m, 1H), 3.23-3.19 (m, |
| | | | carboxamide | | | 1H), 3.07-2.99 (m, 1H), |
| | | | | | | 2.75-2.66 (m, 2H), 2.02 (d, |
| | | | | | | J = 15.8 Hz, 3H), 1.95-1.78 |
| | | | | | | (m, 3H), 1.58-1.47 (m, 1H) |
| 553 | 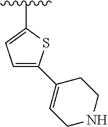 [SMILES: *c1ccc(C2=CCNCC2)s1] | 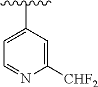 [SMILES: *c1ccnc(C(F)F)c1] | (R)-N-(1-(4- amino-7-(2- (difluoromethyl) pyridin-4- yl)pyrrolo[2,1- f][1,2,4]triazin-5- yl)piperidin-3-yl)- 5-(1,2,3,6-tetra- | 551.2 | E: 1.329 F: 1.016 | (400 MHz, DMSO-d₆) δ ppm 8.69 (d, J = 5.4 Hz, 1H), 8.49 (s, 1H), 8.41 (d, J = 7.6 Hz, 1H), 8.35-8.29 (m, 1H), 8.24-8.07 (m, 1H), 7.99 (s, 1H), 7.76 (d, J = 3.9 Hz, 1H), 7.46 (s, 1H), 7.16 (d, J = 3.7 Hz, 1H), 6.97 (t, J = |
| | | | hydropyridin-4- | | | 55.5 Hz, 2H), 6.45-6.37 (m, |
| | | | yl)thiophene-2- | | | 1H), 4.21-4.11 (m, 1H), 3.96 |
| | | | carboxamide | | | (br s, 2H), 3.25-3.19 (m, 3H), |
| | | | | | | 3.09-3.04 (m, 1H), 2.73 (ddd, |
| | | | | | | J = 2.0, 4.6, 10.5 Hz, 2H), |
| | | | | | | 2.47-2.41 (m, 2H), 1.99-1.77 |
| | | | | | | (m, 4H), 1.59-1.48 (m, 1H) |
| 554 | 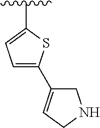 [SMILES: *c1ccc(C2=CCNC2)s1] | 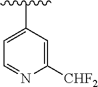 [SMILES: *c1ccnc(C(F)F)c1] | (R)-N-(1-(4- amino-7-(2- (difluoromethyl) pyridin-4-yl)pyrrolo [2,1-f][1,2,4] triazin-5-yl) piperidin-3-yl)- 5-(2,5-dihydro- | 537.2 | E: 1.292 F: 1.001 | (400 MHz, DMSO-d₆) δ ppm 9.36 (br d, J = 5.1 Hz, 1H), 8.69 (d, J = 5.1 Hz, 1H), 8.54- 8.44 (m, 2H), 8.32 (s, 1H), 8.24-8.06 (m, 1H), 8.00 (s, 1H), 7.80 (d, J = 3.7 Hz, 1H), 7.46 (s, 1H), 7.24 (d, J = 3.9 Hz, 1H), 6.97 (t, J = 55.0 Hz, 1H), |
| | | | 1H-pyrrol-3- | | | 6.31 (s, 1H), 4.31 (br d, J = |
| | | | yl)thiophene-2- | | | 1.2 Hz, 2H), 4.14 (br s, 3H), |
| | | | carboxamide | | | 3.26-3.20 (m, 1H), 3.12-3.03 |
| | | | | | | (m, 1H), 2.78-2.69 (m, 2H), |
| | | | | | | 2.00-1.71 (m, 4H), 1.62-1.46 |
| | | | | | | (m, 1H) |

The following examples in Table 40 were prepared from Example 282 through Suzuki coupling using appropriate boronate or boronic acid.

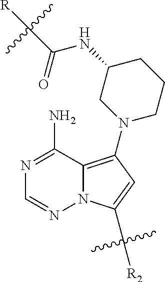

TABLE 40

| | | | | | HPLC | |
|---|---|---|---|---|---|---|
| | | | | | Method, | |
| | | | | LCMS | RT | |
| Example | R | R₂ | Name | (M + H)⁺ | (min.) | ¹H NMR |
| 555 | 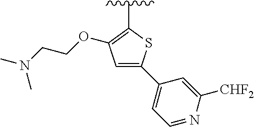 [SMILES: *c1sc(-c2ccnc(C(F)F)c2)cc1OCCN(C)C] | 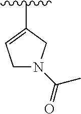 [SMILES: *C1=CCN(C(C)=O)C1] | (R)-N-(1-(7-(1- acetyl-2,5- dihydro-1H- pyrrol-3-yl)-4- aminopyrrolo [2,1-f][1,2,4] triazin-5- yl)piperidin- | 666.2 | E: 1.625 | (400 MHz, DMSO-d₆) δ ppm 8.74 (d, J = 5.3 Hz, 1H), 8.08 (s, 1H), 8.04 (s, 1H), 7.87 (s, 2H), 7.67-7.55 (m, 2H), 7.16- 6.85 (m, 1H), 6.82-6.69 (m, 2H), 4.72-4.64 (m, 1H), 4.53- 4.42 (m, 4H), 4.30-4.24 (m, 1H), 4.19 (br s, 1H), 3.20- |
| | | | 3-yl)-5-(2- | | | 3.14 (m, 2H), 2.98 (br s, |
| | | | (difluoromethyl) | | | 2H), 2.77 (br s, 3H), 2.28 |
| | | | pyridin-4-yl)- | | | (br s, 4H), 2.03 (d, J = |
| | | | 3-(2-(dimethyl- | | | 16.3 Hz, 4H), 1.86 (br s, |
| | | | amino)ethoxy) | | | 4H), 1.53 (s, 1H) |
| | | | thiophene-2- | | | |
| | | | carboxamide | | | |
| 556 | 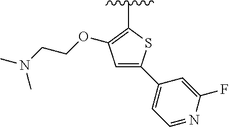 [SMILES: *c1sc(-c2ccnc(F)c2)cc1OCCN(C)C] | 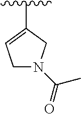 [SMILES: *C1=CCN(C(C)=O)C1] | (R)-N-(1-(7-(1- acetyl-2,5- dihydro-1H- pyrrol-3-yl)-4- aminopyrrolo [2,1-f][1,2,4] triazin-5- yl)piperidin- | 634.2 | E: 1.590 F: 1.021 | (400 MHz, DMSO-d₆) δ ppm 8.30 (d, J = 5.3 Hz, 1H), 8.02 (s, 2H), 7.87 (s, 1H), 7.79 (br s, 1H), 7.68 (br d, J = 5.0 Hz, 1H), 7.58 (s, 1H), 6.81-6.69 (m, 2H), 4.69 (br d, J = 0.8 Hz, 1H), 4.56- 4.38 (m, 4H), 4.30-4.24 (m, |
| | | | 3-yl)-3-(2- | | | 1H), 4.23-4.14 (m, 1H), |
| | | | (dimethylamino) | | | 3.18 (br d, J = 1.3 Hz, 1H), |
| | | | ethoxy)-5-(2- | | | 3.03-2.94 (m, 1H), 2.77 |
| | | | fluoropyridin-4- | | | (br s, 3H), 2.30 (br d, J = |
| | | | yl)thiophene-2- | | | 2.5 Hz, 6H), 2.03 (d, J = |
| | | | carboxamide | | | 16.3 Hz, 3H), 1.96-1.78 |
| | | | | | | (m, 4H), 1.52 (br s, 1H) |

### Example 557

### Preparation of (R)—N-(1-(4-amino-6-cyano-7-(4-(methylsulfonyl)phenyl)pyrrolo[2,1-f][1,2,4]triazin-5-yl)piperidin-3-yl)-5-methylthiophene-2-carboxamide

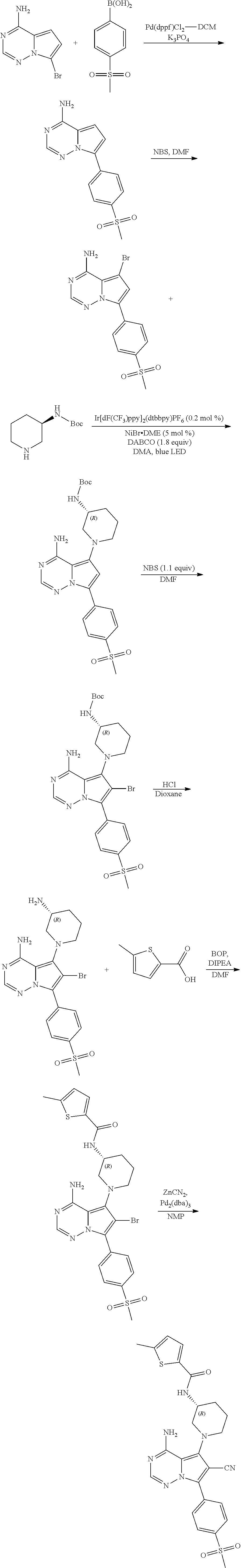

### Example 557A. 7-(4-(Methylsulfonyl)phenyl)pyrrolo[2,1-f][1,2,4]triazin-4-amine

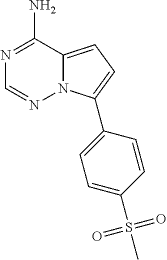 [SMILES: CS(=O)(=O)c1ccc(-c2ccc3c(N)ncnn23)cc1]

7-Bromopyrrolo[2,1-f][1,2,4]triazin-4-amine (2.00 g, 9.39 mmol), (4-(methylsulfonyl)phenyl)boronic acid (1.97 g, 9.86 mmol) and PdCl₂(dppf)-CH₂Cl₂ adduct (383 mg, 0.469 mmol) and phosphoric acid potassium salt (3.99 g, 18.8 mmol) were taken up in THF (60 mL) and water (37.6 mL). The reaction mixture was degassed with Ar and heated at 100° C. for 4 h. After cooling, it was diluted with EtOAc (100 mL) and water (50 mL) was added. Organic layer was separated, concentrated and the residue was purified by flash chromatography to give Example 557A (2.015 g, 74% yield) as an off-white solid. MS: [M+H]⁺=289.0; ¹H NMR (500 MHz, DMSO-d₆) δ ppm 8.38 (d, J=8.8 Hz, 2H), 8.00 (s, 1H), 7.98 (d, J=8.8 Hz, 2H), 7.89 (br s, 2H), 7.25 (d, J=4.4 Hz, 1H), 7.06 (d, J=4.7 Hz, 1H), 3.24 (s, 3H).

### Example 557B. 5-Bromo-7-(4-(methylsulfonyl)phenyl)pyrrolo[2,1-f][1,2,4]triazin-4-amine

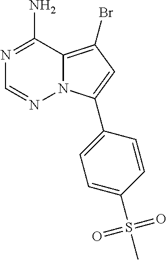 [SMILES: CS(=O)(=O)c1ccc(-c2cc(Br)c3c(N)ncnn23)cc1]

To a solution of Example 557A (500 mg, 1.73 mmol) in DMF (16 mL) at rt, NBS (340 mg, 1.91 mmol) was added. The mixture was stirred at rt for 1 h and 150 mL 50% aq. NaHCO₃ and 10 mL sat. Na₂SO₃ were added. The suspension was stirred at rt for 2 h and filtered. The solids separated were rinsed with H₂O (3×5 mL) and Et₂O (1×5 mL) and dried under vacuum to afford Example 557B (564 mg, 89% yield) as a grey solid. MS: [M+H]⁺=367.0; ¹H NMR (500 MHz, DMSO-d₆) δ ppm 8.36-8.30 (m, 2H), 8.02 (s, 1H), 8.01-7.98 (m, 2H), 7.45 (s, 1H), 3.25 (s, 3H).

### Example 557C. tert-butyl (R)-(1-(4-amino-7-(4-(methylsulfonyl)phenyl)pyrrolo[2,1-f][1,2,4]triazin-5-yl)piperidin-3-yl)carbamate

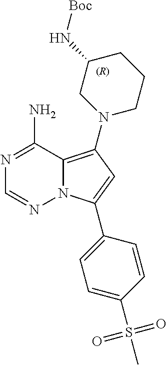

Example 557B (150 mg, 0.408 mmol), Ir[dF(CF₃)ppy]₂(dtbbpy)PF₆ (9.2 mg, 8.2 μmol), NiBr₂-DME (12.6 mg, 0.041 mmol), DABCO (165 mg, 1.47 mmol) and tert-butyl piperidin-3-ylcarbamate (491 mg, 2.45 mmol) were taken in DMA (5 mL). The reaction mixture was degassed with N₂ and stirred under blue LED irradiation at 50° C. for 18 h. The reaction mixture was diluted with EtOAc (200 mL) brine (1×50 mL) solution was added. Organic layer was separated, dried over Na₂SO₄, concentrated and the residue was purified by silica-gel flash chromatography (20-100% EtOAc/DCM) to give Example 557C (97 mg, 49% yield) as a white solid. MS: [M+H]⁺=487.0; ¹H NMR (500 MHz, DMSO-d₆) δ ppm 8.33 (br d, J=8.2 Hz, 2H), 7.94 (br d, J=8.5 Hz, 2H), 7.87 (s, 1H), 7.16 (s, 1H), 7.10-6.92 (m, 1H), 3.68 (br d, J=4.6 Hz, 2H), 3.20 (s, 3H), 3.13-3.02 (m, 1H), 2.95-2.84 (m, 1H), 1.89-1.73 (m, 2H), 1.71-1.56 (m, 1H), 1.35 (s, 9H).

### Example 557D. tert-butyl (R)-(1-(4-amino-6-bromo-7-(4-(methylsulfonyl)phenyl)pyrrolo[2,1-f][1,2,4]triazin-5-yl)piperidin-3-yl)carbamate

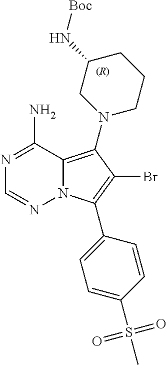

To a solution of Example 557C (200 mg, 0.411 mmol) in DCM (5 mL), was added NBS (80 mg, 0.452 mmol) at rt. Reaction was allowed stir at rt for 2 h. The mixture was diluted with DCM, washed with water and brine solution. Organic layer was separated, dried over Na₂SO₄, filtered and concentrated to afford brown solid, which was purified by silica-gel chromatography (0-100% EtOAc in DCM) which afforded example 557D (80 mg, 34% yield) as a yellow solid. MS: [M+H]⁺=567.0; ¹H NMR (400 MHz, DMSO-d₆) δ ppm 8.26-8.19 (m, 1H), 8.03 (d, J=8.0 Hz, 2H), 7.90 (d, J=8.5 Hz, 2H), 7.80 (s, 1H), 7.49-7.43 (m, 1H), 6.99-6.89 (m, 1H), 3.59-3.35 (m, 6H), 3.21-3.07 (m, 1H), 3.03-2.94 (m, 1H), 1.92-1.66 (m, 2H), 1.36 (s, 10H), 1.28-1.19 (m, 1H).

### Example 557E. (R)-5-(3-aminopiperidin-1-yl)-6-bromo-7-(4-(methylsulfonyl)phenyl)pyrrolo[2,1-f][1,2,4]triazin-4-amine.HCl

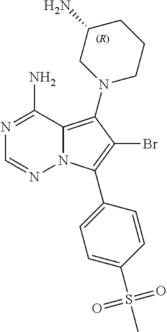

To a solution of Example 557D (80 mg, 0.141 mmol) in dioxane (1 mL), was added 4N HCl in dioxane (0.043 mL, 1.42 mmol). The mixture was stirred at rt for 2 h and concentrated to afford Example 557E (85 mg, 98% yield) as a yellow solid. MS: [M+H]⁺=567.0; ¹H NMR (400 MHz, DMSO-d₆) δ ppm 8.25-8.15 (m, 2H), 8.06 (s, 2H), 7.91 (s, 2H), 7.66-7.54 (m, 1H), 3.80-3.61 (m, 4H), 3.53-3.38 (m, 2H), 3.31 (s, 3H), 3.12-2.97 (m, 1H), 2.13-2.02 (m, 1H), 1.88-1.75 (m, 1H), 1.48-1.28 (m, 2H).

### Example 557F

### Preparation of (R)—N-(1-(4-amino-6-bromo-7-(4-(methylsulfonyl)phenyl)pyrrolo[2,1-f][1,2,4]triazin-5-yl)piperidin-3-yl)-5-methylthiophene-2-carboxamide

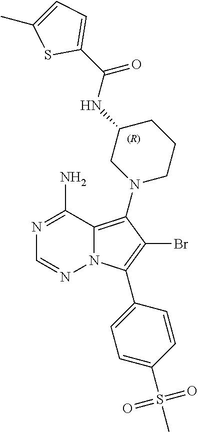

To a solution of Example 557E (86 mg, 0.141 mmol) and 5-methylthiophene-2-carboxylic acid (20 mg, 0.141 mmol) in DMF (2 mL), were added BOP (93 mg, 0.211 mmol) and DIEA (0.123 mL, 0.703 mmol). The mixture was stirred at rt for 2 h and quenched with ice cold water and stirred gently for 5 minutes. Precipitated solid was filtered, washed with water and dried under vacuum. Crude solid was purified by preparative HPLC to afford Example 557F (8.8 mg, 10% yield) as a pale yellow solid. LC-MS Method E: RT=1.750 min, [M+H]⁺=589.1; LC-MS Method F: RT=1.589 min, [M+H]⁺=589.2; ¹H NMR (400 MHz, DMSO-d₆) δ ppm 8.31-8.16 (m, 2H), 8.03 (d, J=8.6 Hz, 2H), 7.91 (d, J=8.6 Hz, 2H), 7.81 (s, 1H), 7.59 (d, J=3.7 Hz, 1H), 7.56-7.43 (m, 1H), 6.82 (d, J=2.9 Hz, 1H), 4.10-3.98 (m, 1H), 3.28 (s, 3H), 3.20-3.16 (m, 1H), 3.08-2.98 (m, 2H), 2.44 (s, 3H), 1.99-1.91 (m, 2H), 1.87-1.70 (m, 2H), 1.51-1.36 (m, 1H).

### Example 557

### Preparation of (R)—N-(1-(4-amino-6-cyano-7-(4-(methylsulfonyl)phenyl)pyrrolo[2,1-f][1,2,4]triazin-5-yl)piperidin-3-yl)-5-methylthiophene-2-carboxamide

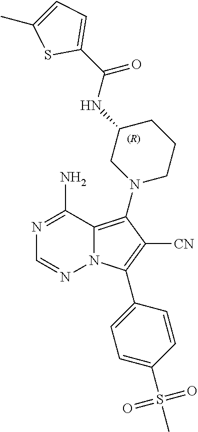

To a suspension of Example 282F (40 mg, 0.068 mmol), dppf (18.8 mg, 0.034 mmol) and Zinc Cyanide (15.9 mg, 0.136 mmol) in NMP (2 mL), was added Pd₂(dba)₃ (18.6 mg, 0.020 mmol). The mixture was heated at 120° C. for 14 h. After cooling to rt, it was diluted with ethyl acetate (20 mL) followed by the addition of water (30 mL). Organic layer was separated, dried over Na₂SO₄, filtered, concentrated and the residue was purified by preparative HPLC to afford Example 557 (7 mg, 19% yield) as a pale yellow solid. LC-MS Method G: RT=7.718 min, Method H: RT=8.753 min; LC-MS: [M+H]⁺=536; ¹H NMR (400 MHz, DMSO-d₆) δ ppm 8.60 (br s, 1H), 8.30 (d, J=8.0 Hz, 1H), 8.16-8.01 (m, 4H), 7.96 (s, 1H), 7.61 (br s, 1H), 7.44 (br s, 1H), 6.83 (dd, J=3.5, 1.0 Hz, 1H), 4.24-4.09 (m, 1H), 3.30 (s, 3H), 3.23-3.05 (m, 3H), 2.45 (s, 3H), 2.02-1.80 (m, 3H), 1.63-1.36 (m, 2H).

The following examples in Table 41 were prepared using the same procedure as shown in Example 557 (through steps: D, E & F).

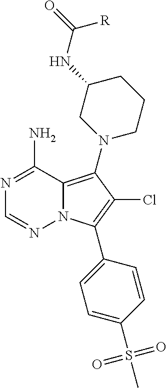

TABLE 41

| | | | | HPLC | |
|---|---|---|---|---|---|
| | | | LCMS | Method, | |
| Example | R | Name | (M + H)⁺ | RT (min.) | ¹H NMR |
| 558 | 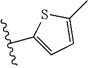 [SMILES: *c1ccc(C)s1] | (R)-N-(1-(4-amino-6-chloro-7-(4- (methylsulfonyl)phenyl)pyrrolo[2,1- f][1,2,4]triazin-5-yl)piperidin-3-yl)- 5-methylthiopbene-2-carboxamide | 545.1 | E: 1.520 F: 1.742 | (400 MHz, DMSO-d6) δ 8.29-8.16 (m, 2H), 8.08-8.01 (m, 2H), 7.99-7.92 (m, 2H), 7.86 (s, 1H), 7.59 (d, J = 3.7 Hz, 1H), 7.46 (dd, J = 2.2, 1,2 Hz, 1H), 6.82 (d, J = 3.2 Hz, 1H), 4.11- |
| | | | | | 4.00 (m, 1H), 3.29-3.24 (m, 4H), |
| | | | | | 3.22-3.18 (m, 1H), 3.12-3.03 (m, |
| | | | | | 1H), 2.44 (s, 3H), 1.99-1.92 (m, 1H), |
| | | | | | 1.86-1.69 (m, 2H), 1.51-1.39 (m, 2H) |
| 559 | 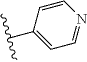 [SMILES: *c1ccncc1] | (R)-N-(1-(4-amino-6-chloro-7-(4- (methylsulfonyl)phenyl)pyrrolo[2,1- f][1,2,4]triazin-5-yl)piperidin-3- yl)isonicotinamide | 526.1 | E: 1.414 F: 1.069 | (400 MHz, DMSO-d6) δ 8.76-8.68 (m, 2H), 8.68-8.58 (m, 1H), 8.26 (br s, 1H), 8.08-8.01 (m, 2H), 8.00- 7.93 (m, 2H), 7.86 (s, 1H), 7.74 (d, |
| | | | | | J = 5.9 Hz, 2H), 7.48 (dt, J = 3.8, |
| | | | | | 1.8 Hz, 1H), 4.20-4.10 (m, 1H), 3.90 |
| | | | | | (s, 1H), 3.28-3.24 (m, 5H), 3.11-3.05 |
| | | | | | (m, 1H), 2.02-1.95 (m, 1H), 1.88- |
| | | | | | 1.73 (m, 2H), 1.53-1.42 (m, 1H) |

### Example 561

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpiperidin-4-yl)oxy)thiophene-2-carboxamide

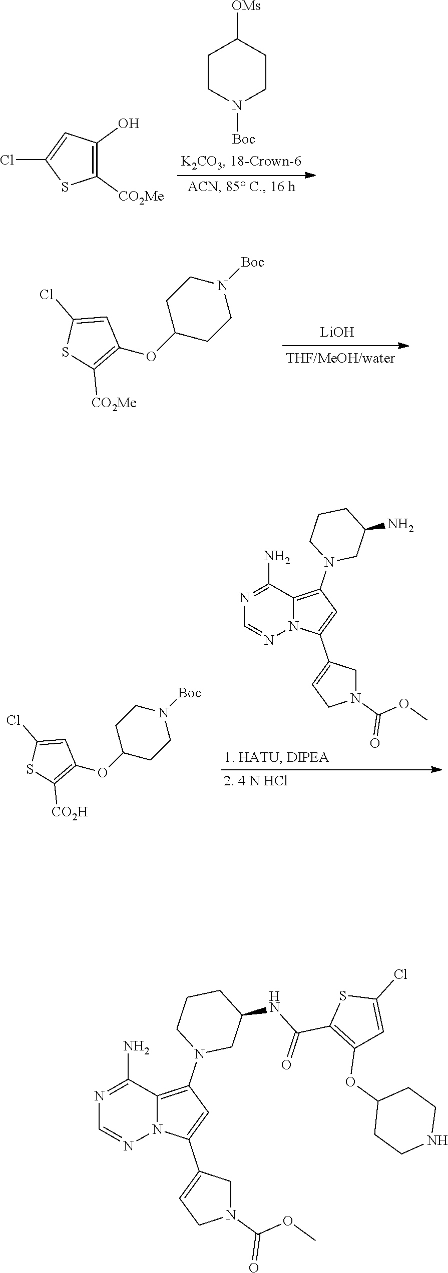

### Example 561A. tert-butyl 4-((5-chloro-2-(methoxycarbonyl)thiophen-3-yl)oxy)piperidine-1-carboxylate

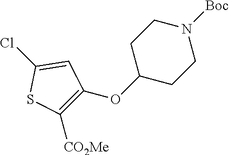 [SMILES: COC(=O)c1sc(Cl)cc1OC1CCN(C(=O)OC(C)(C)C)CC1]

Analogous to the procedure for the preparation of Example 346A, reaction of methyl 5-chloro-3-hydroxythiophene-2-carboxylate (1.1 g, 5.71 mmol) and tert-butyl 4-((methylsulfonyl)oxy)piperidine-1-carboxylate (3.99 g, 14.3 mmol) afforded Example 561A (1.26 g, 59% yield) as a yellow thick liquid. MS: [M+H-t-Bu]⁺=320.0; ¹H NMR (400 MHz, DMSO-d₆) δ ppm 7.42 (s, 1H), 4.69 (td, J=3.3, 6.9 Hz, 1H), 3.73 (s, 3H), 3.57-3.45 (m, 2H), 3.37-3.33 (m, 2H), 1.83 (dt, J=3.8, 8.6 Hz, 2H), 1.66-1.53 (m, 2H), 1.41 (s, 9H).

### Example 561B. 3-((1-(tert-butoxycarbonyl)piperidin-4-yl)oxy)-5-chlorothiophene-2-carboxylate.Lithium salt

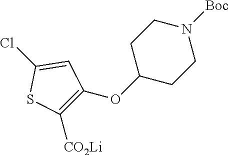

Analogous to the procedure for the preparation of Example 346B, reaction of Example 561A (1.26 g, 3.35 mmol) and lithium hydroxide monohydrate (0.281 g, 6.70 mmol) afforded Example 561B (1.1 g, 89% yield) as a yellow solid. MS: [M+H-t-Bu]⁺=306.0; ¹H NMR (400 MHz, DMSO-d₆) δ ppm 6.85 (s, 1H), 4.70-4.63 (m, 1H), 3.69-3.58 (m, 2H), 3.22-3.08 (m, 2H), 1.80-1.68 (m, 2H), 1.57-1.44 (m, 2H), 1.40 (s, 9H).

### Example 560C. tert-butyl (R)-4-((2-((1-(4-amino-7-(1-(methoxycarbonyl)-2,5-dihydro-1H-pyrrol-3-yl)pyrrolo[2,1-f][1,2,4]triazin-5-yl)piperidin-3-yl)carbamoyl)-5-chlorothiophen-3-yl)oxy)piperidine-1-carboxylate

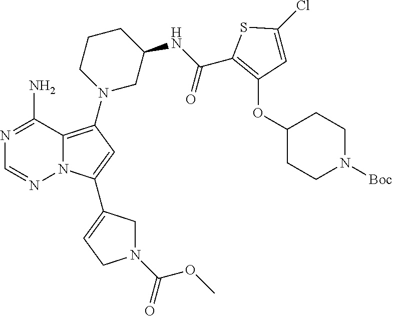 [SMILES: COC(=O)N1CC=C(c2cc(N3CCC[C@@H](NC(=O)c4sc(Cl)cc4OC4CCN(C(=O)OC(C)(C)C)CC4)C3)c3c(N)ncnn23)C1]

To a solution of Example 561B (321 mg, 0.816 mmol) and Intermediate 31 (300 mg, 0.816 mmol) in DMF (10 mL). HATU (372 mg, 0.979 mmol) and DIEA (0.427 mL, 2.45 mmol) were added. The mixture was stirred at rt for 2 h and cold water was added followed by stirring for 5 min. Precipitated solid was filtered, washed with water and dried under vacuum to afford Example 561C (500 mg, 87% yield) as a yellow solid. MS: [M+H]⁺=701.2 ¹H NMR (400 MHz, DMSO-d₆) δ ppm 7.85-7.82 (m, 2H), 7.43 (s, 1H), 7.39-7.26 (m, 1H), 6.73 (s, 2H), 4.77-4.66 (m, 1H), 4.58-4.49 (m, 2H), 4.37-4.23 (m, 2H), 4.19-4.08 (m, 1H), 3.65 (d, J=3.5 Hz, 4H), 3.46-3.38 (m, 3H), 3.25-2.88 (m, 5H), 2.84-2.71 (m, 1H), 2.04-1.70 (m, 4H), 1.65-1.45 (m, 3H), 1.35 (s, 9H).

### Example 561

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpiperidin-4-yl)oxy)thiophene-2-carboxamide

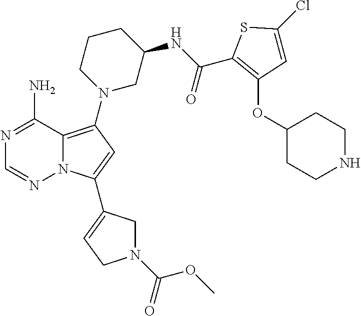 [SMILES: COC(=O)N1CC=C(c2cc(N3CCC[C@@H](NC(=O)c4sc(Cl)cc4OC4CCNCC4)C3)c3c(N)ncnn23)C1]

To a solution of Example 561C (500 mg, 0.713 mmol) in dioxane (8 mL), was added 4N HCl in dioxane (0.891 mL, 3.57 mmol). The reaction mixture was stirred at rt for 4 h, concentrated and the residue was purified by preparative HPLC to afford Example 561 (220 mg, 49% yield) as a white solid. LCMS Method G: RT=4.317 min; Method H: RT=5.398 min; MS: [M+H]⁺=601.3; ¹H NMR (400 MHz, DMSO-d₆) δ ppm 8.05 (br s, 1H), 7.86 (s, 1H), 7.51-7.32 (m, 2H), 6.85-6.66 (m, 3H), 4.65-4.46 (m, 3H), 4.29 (dd, J=6.8, 3.3 Hz, 2H), 4.24-4.10 (m, 2H), 3.66 (d, J=4.5 Hz, 3H), 3.20-3.07 (m, 1H), 2.98-2.83 (m, 4H), 2.64-2.54 (m, 2H), 2.14-1.75 (m, 5H), 1.59-1.38 (m, 4H).

### Example 562

### Preparation of methyl (R)-3-(4-amino-5-(3-(5-chloro-3-((1-methylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

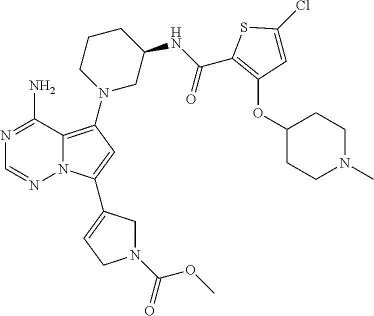 [SMILES: COC(=O)N1CC=C(c2cc(N3CCC[C@@H](NC(=O)c4sc(Cl)cc4OC4CCN(C)CC4)C3)c3c(N)ncnn23)C1]

Analogous to the procedure for the preparation of Example 375, reaction of Example 51 (25 mg, 0.042 mmol) and formaldehyde solution (38%, 0.008 mL, 0.100 mmol) afforded Example 562 (5.1 mg, 20% yield) as a pale yellow solid. LC-MS Method E: RT=1.627 min, [M+H]⁺=615.2; LC-MS Method F: RT=1.065 min, [M+H]⁺=615.2; ¹H NMR (400 MHz, DMSO-d₆) δ ppm 8.06 (br s, 1H), 7.86 (s, 1H), 7.40 (s, 2H), 6.77-6.68 (m, 3H), 4.66-4.57 (m, 1H), 4.57-4.45 (m, 2H), 4.36-4.26 (m, 2H), 4.22-4.09 (m, 1H), 3.66 (d, J=4.2 Hz, 3H), 3.19-2.91 (m, 3H), 2.86-2.58 (m, 4H), 2.46 (s, 3H), 2.32-2.19 (m, 3H), 2.07-1.96 (m, 2H), 1.94-1.69 (m, 3H), 1.53 (br s, 1H).

### Example 563

### Preparation of methyl (R)-3-(4-amino-5-(3-(5-chloro-3-((1-isopropylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

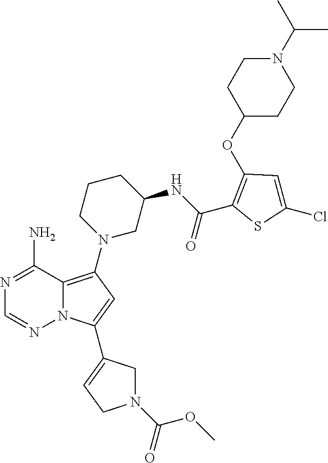 [SMILES: COC(=O)N1CC=C(c2cc(N3CCC[C@@H](NC(=O)c4sc(Cl)cc4OC4CCN(C(C)C)CC4)C3)c3c(N)ncnn23)C1]

Analogous to the procedure for the preparation of Example 437A, reaction of Example 561 (25 mg, 0.042 mmol) and acetone (5 μl, 0.06 mmol) afforded Example 563 (12.6 mg, 47% yield). LC-MS Method E: RT=1.761 min, [M+H]⁺=643.2; LC-MS Method F: RT=1.177 min, [M+H]⁺=643.3; ¹H NMR (400 MHz, DMSO-d₆) δ ppm 8.03 (br s, 1H), 7.86 (s, 1H), 7.38 (s, 2H), 6.77-6.70 (m, 3H), 4.66-4.50 (m, 3H), 4.30 (br s, 2H), 4.16 (br s, 1H), 3.66 (d, J=3.8 Hz, 4H), 3.18 (br d, J=5.3 Hz, 2H), 2.93 (br s, 2H), 2.79-2.64 (m, 3H), 2.85-2.58 (m, 1H), 2.01-1.86 (m, 3H), 1.81 (br s, 4H), 1.53 (br s, 1H), 0.94 (br s, 6H).

### Example 564

### Preparation of methyl (R)-3-(4-amino-5-(3-(5-chloro-3-((1-(2-hydroxyethyl)piperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

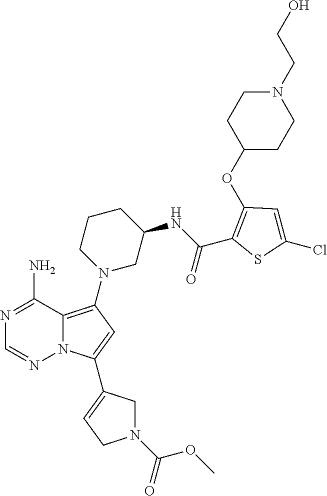 [SMILES: COC(=O)N1CC=C(c2cc(N3CCC[C@@H](NC(=O)c4sc(Cl)cc4OC4CCN(CCO)CC4)C3)c3c(N)ncnn23)C1]

To a solution of Example 561 (25 mg, 0.042 mmol) in DCM (1 mL), were added TEA (0.017 mL, 0.125 mmol) and 2-bromoethan-1-ol (6.8 mg, 0.054 mmol). Reaction mixture was stirred at 50° C. for 14 h, concentrated and the residue was purified by preparative HPLC to afford Example 564 (14.4 mg, 54% yield). LC-MS Method E: RT=1.619 min, [M+H]⁺=645.2; LC-MS Method F: RT=1.109 min, [M+H]⁺=645.3; ¹H NMR (400 MHz, DMSO-d₆) δ ppm 8.03 (br s, 1H), 7.86 (s, 1H), 7.40 (s, 2H), 6.77-6.66 (m, 3H), 4.65-4.41 (m, 4H), 4.34-4.13 (m, 3H), 3.66 (d, J=4.2 Hz, 3H), 3.51-3.43 (m, 2H), 3.19-3.12 (m, 1H), 2.98-2.73 (m, 3H), 2.65-2.59 (m, 2H), 2.46-2.33 (m, 2H), 2.04-1.78 (m, 6H), 1.76-1.64 (m, 2H), 1.59-1.47 (m, 2H).

### Example 565

### Preparation of methyl (R)-3-(5-(3-(3-((1-acetylpiperidin-4-yl)oxy)-5-chlorothiophene-2-carboxamido)piperidin-1-yl)-4-aminopyrrolo[2,1-f][1,2,4]triazin-7-yl)-2,5-dihydro-1H-pyrrole-1-carboxylate

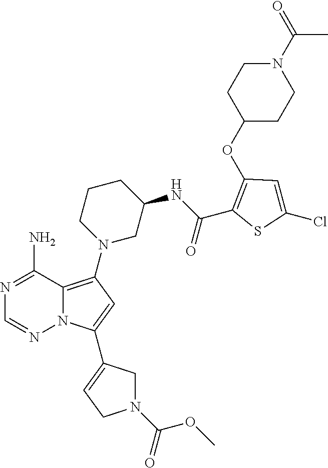 [SMILES: COC(=O)N1CC=C(c2cc(N3CCC[C@@H](NC(=O)c4sc(Cl)cc4OC4CCN(C(C)=O)CC4)C3)c3c(N)ncnn23)C1]

To a solution of Example 561 (30 mg, 0.050 mmol) in DCM (2 mL), were added TEA (0.014 mL, 0.100 mmol) and acetic anhydride (4 μl, 0.04 mmol). The mixture was stirred at rt for 30 min. It was quenched with sat. NaHCO₃ and extracted with DCM. DCM layer was concentrated, dried over Na₂SO₄, filtered and concentrated. The residue was purified by preparative HPLC to afford Example 565 (18 mg, 56% yield) as a white solid. LCMS Method G: RT=5.588 min; Method H: RT=8.150 min; MS: [M+H]⁺=643.2; ¹H NMR (400 MHz, DMSO-d₆) δ ppm 8.03 (br s, 1H), 7.84 (s, 1H), 7.40 (s, 2H), 6.71 (d, J=4.5 Hz, 3H), 4.79-4.64 (m, 1H), 4.52 (br d, J=12.0 Hz, 2H), 4.34-4.24 (m, 2H), 4.16-4.06 (m, 1H), 3.89-3.74 (m, 1H), 3.69-3.62 (m, 3H), 3.34-3.04 (m, 3H), 2.97-2.72 (m, 3H), 2.53-2.49 (m, 3H), 2.07-1.86 (m, 5H), 1.82-1.47 (m, 4H).

### Example 566

### Preparation of methyl (R)-3-(4-amino-5-(3-(5-chloro-3-((1-(2,2-difluoroethyl)piperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

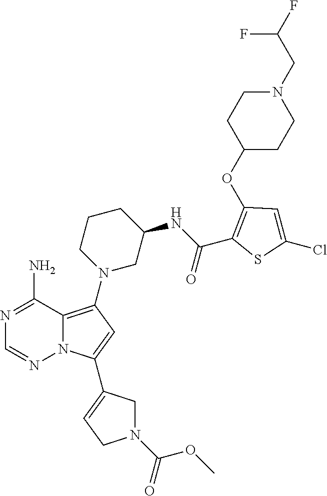 [SMILES: COC(=O)N1CC=C(c2cc(N3CCC[C@@H](NC(=O)c4sc(Cl)cc4OC4CCN(CC(F)F)CC4)C3)c3c(N)ncnn23)C1]

To a solution of Example 561 (30 mg, 0.050 mmol) in acetonitrile (2 mL), were added K₂COS (13.8 mg, 0.100 mmol) and 1,1-difluoro-2-iodoethane (7 μl, 0.08 mmol). Reaction mixture was heated at 80° C. for 16 h. After cooling, it was diluted with ethyl acetate followed by the addition of water. Organic layer was separated, concentrated and the residue was purified by preparative HPLC to afford Example 566 (15.6 mg, 47% yield) as a pale yellow solid. LC-MS Method E: RT=1.957 min, [M+H]⁺=665.2; LC-MS Method F: RT=1.141 min, [M+H]⁺=665.3; ¹H NMR (400 MHz, DMSO-d₆) δ ppm 8.15-7.91 (m, 2H), 7.86 (s, 1H), 7.41 (s, 1H), 6.86-6.61 (m, 3H), 6.25-5.93 (m, 1H), 4.66-4.45 (m, 4H), 4.30 (br dd, J=2.3, 6.2 Hz, 2H), 4.22-4.11 (m, 1H), 3.66 (d, J=3.7 Hz, 4H), 3.15 (br d, J=2.4 Hz, 1H), 2.96-2.89 (m, 1H), 2.86-2.67 (m, 6H) 2.03-1.61 (m, 7H), 1.57-1.43 (m, 1H). ¹⁹F NMR (400 MHz, DMSO-d₆) δ ppm=−118.8.

The following examples in Table 42 were prepared using the same procedures as shown in Example 561 and 562.

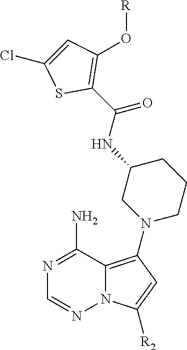

TABLE 42

| | | | | | HPLC | |
|---|---|---|---|---|---|---|
| | | | | LCMS | Method, | |
| Example | R | R₂ | Name | (M + H)⁺ | RT (min.) | ¹H NMR |
| 567 | 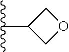 [SMILES: *C1COC1] | 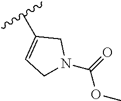 [SMILES: *C1=CCN(C(=O)OC)C1] | Methyl (R)-3-(4- amino-5-(3-(5- chloro-3-(oxetan- 3-yloxy)thiophene- 2-carboxamido) piperidin-1-yl) pyrrolo[2,1-f] | 574.2 | G: 6.453 K: 7.598 | (400 MHz, DMSO-d₆) δ ppm 8.04- 7.92 (m, 1H), 7.85 (s, 1H), 7.44 (br s, 1H), 7.04 (s, 1H), 6.84-6.60 (m, 3H), 5.44 (quin, J = 5.2 Hz, 1H), 4.89 (q, J = 6.6 Hz. 2H), 4.66-4.48 (m, 4H), 4.29 (br s, 2H), 4.17 (br s, 1H), 3.65 (d, |
| | | | [1,2,4]triazin-7- | | | J = 4.3 Hz, 3H), 3.52-3.34 (m, |
| | | | yl)-2,5-dihydro- | | | 4H), 3.09 (br s, 1H), 2.98-2.77 |
| | | | 1H-pyrrole-1- | | | (m, 3H), 2.67-2.61 (m, 4H), |
| | | | carboxylate | | | 1.93-1.56 (m, 5H) |
| 568 | 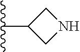 [SMILES: *C1CNC1] | 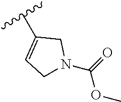 [SMILES: *C1=CCN(C(=O)OC)C1] | Methyl (R)-3-(4- amino-5-(3-(3- (azetidin-3- yloxy)-5-chloro- thiophene-2- carboxamido) piperidin-1-yl) | 573.2 | G: 4.161 K: 5.319 | (400 MHz, DMSO-d₆) δ ppm 8.24- 8.02 (m, 1H), 7.85 (s, 1H), 7.62- 7.35 (m, 1H), 7.07 (s, 1H), 6.89- 6.52 (m, 3H), 5.25-5.10 (m, 1H), 4.53 (br d, J = 11.4 Hz, 2H), 4.18 (br s, 4H), 3.86-3.74 (m, 2H), 3.65 (d, J = 4.5 Hz, 4H), 3.58-3.41 (m, |
| | | | pyrrolo[2,1-f] | | | 3H), 3.20-2.97 (m, 2H), 2.96-2.68 |
| | | | [1,2,4]triazin-7- | | | (m, 3H), 2.59-2.53 (m, 4H), 1.83 |
| | | | yl)-2,5-dihydro- | | | (br d, J = 10.1 Hz, 5H) |
| | | | 1H-pyrrole-1- | | | |
| | | | carboxylate | | | |
| 569 | 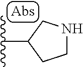 [SMILES: *C1CCNC1] | 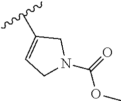 [SMILES: *C1=CCN(C(=O)OC)C1] | Methyl 3-(4- amino-5-((3R)- 3-(5-chloro-3- (pyrrolidin-3- yloxy)thiophene- 2-carboxamido) piperidin-1-yl) | 587.2 | G: 4.274 K: 5.455 | (400 MHz, DMSO-d₆) δ ppm 8.04 (br s, 1H), 7.85 (s, 1H), 7.57 (br d, J = 7.5 Hz, 1H), 7.32 (s, 1H), 6.86- 6.62 (m, 3H), 5.12-4.98 (m, 1H), 4.53 (br d, J = 11.1 Hz, 2H), 4.29 (br s, 2H), 4.12 (br s, 1H), 3.65 (d, J = 4.3 Hz, 3H), 3.58-3.42 (m, 1H), |
| | | | pyrrolo[2,1- | | | 3.10 (br s, 2H), 3.00-2.72 (m, 6H), |
| | | | f][1,2,4]triazin- | | | 2.56-2.52 (m, 3H), 2.33-2.45 (m, |
| | | | 7-yl)-2,5-dihydro- | | | 3H), 2.21-2.31 (m, 1H), 2.03 (dt, |
| | | | 1H-pyrrole-1- | | | J = 7.1, 14.3 Hz, 1H), 1.93-1.73 |
| | | | carboxylate | | | (m, 6H), 1.52 (br s, 1H) |
| 570 | 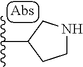 [SMILES: *C1CCNC1] | 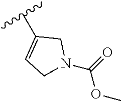 [SMILES: *C1=CCN(C(=O)OC)C1] | Methyl 3-(4- amino-5-((3R)- 3-(5-chloro-3- (pyrrolidin-3- yloxy)thiophene- 2-carboxamido) piperidin-1-yl) | 587.2 | G: 4.272 K: 5.460 | (400 MHz, DMSO-d₆) δ ppm 8.12- 7.91 (m, 1H), 7.85 (s, 1H), 7.54 (br s, 1H), 7.32 (s, 1H), 6.89-6.57 (m, 3H), 5.07-5.02 (m, 1H), 4.54 (br d, J = 11.5 Hz, 2H), 4.36-4.23 (m, 2H), 4.13 (br s, 1H), 3.66 (d, J = 4.4 Hz, 3H), 3.58-3.49 (m, 3H), |
| | | | pyrrolo[2,1- | | | 3.09 (br s, 2H), 3.04-2.84 (m, 5H), |
| | | | f][1,2,4]triazin- | | | 2.84-2.73 (m, 2H), 2.59-2.53 (m, |
| | | | 7-yl)-2,5- | | | 3H), 2.22-1.93 (m, 2H), 1.92-1.74 |
| | | | dihydro- | | | (m, 6H), 1.53 (br s, 1H) |
| | | | 1H-pyrrole-1- | | | |
| | | | carboxylate | | | |
| 571 | 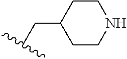 [SMILES: *CC1CCNCC1] | 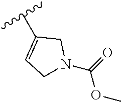 [SMILES: *C1=CCN(C(=O)OC)C1] | Methyl (R)-3- (4-amino-5- (3-(5-chloro- 3-(piperidin-4- ylmethoxy) thiophene-2- carboxamido) | 615.2 | E: 1.386 F: 1.105 | (400 MHz, DMSO-d6) δ ppm 8.12- 7.91 (m, 1H),7.85 (s, 1H), 7.46 (br s, 1H), 7.35 (s, 1H), 6.73 (br d, J = 2.0 Hz, 3H), 4.59-4.40 (m, 2H), 4.35-4.00 (m, 4H), 3.65 (d, J = 4.3 Hz, 3H), 3.17-3.12 (m, 3H), 2.98-2.78 (m, 5H), 2.39-2.29 (m, |
| | | | piperidin-1-yl) | | | 2H), 1.86-1.75 (m, 4H), 1.69-1.53 |
| | | | pyrrolo[2,1-f] | | | (m, 3H), 1.20-1.05 (m, 2H) |
| | | | [1,2,4]triazin-7- | | | |
| | | | yl)-2,5-dihydro- | | | |
| | | | 1H-pyrrole-1- | | | |
| | | | carboxylate | | | |
| 572 | 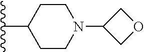 [SMILES: *C1CCN(C2COC2)CC1] | 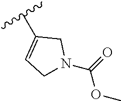 [SMILES: *C1=CCN(C(=O)OC)C1] | Methyl (R)-3- (4-amino-5-(3- (5-chloro-3-((1- (oxetan-3-yl) piperidin-4-yl) oxy)thiophene- 2-carboxamido) | 657.2 | E: 1.774 F: 1.107 | (400 MHz, DMSO-d6) δ ppm 8.07- 7.93 (m, 1H), 7.86 (s, 1H), 7.40 (s, 1H), 7.37-7.28 (m, 1H), 6.74 (br s, 3H), 4.65-4.45 (m, 4H), 4.42-4.24 (m, 4H), 4.19-4.08 (m, 1H), 3.66 (d, J = 3.9 Hz, 3H), 3.23-3.07 (m, 3H), 3.00-2.72 (m, 3H), 2.34 (br s, |
| | | | piperidin-1-yl) | | | 3H), 2.19-2.01 (m, 3H), 1.99-1.88 |
| | | | pyrrolo[2,1-f] | | | (m, 3H), 1.85-1.65 (m, 3H) |
| | | | [1,2,4]triazin-7- | | | |
| | | | yl)-2,5-dihydro- | | | |
| | | | 1H-pyrrole-1- | | | |
| | | | carboxylate | | | |
| 573 | 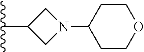 [SMILES: *C1CN(C2CCOCC2)C1] | 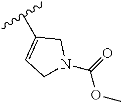 [SMILES: *C1=CCN(C(=O)OC)C1] | Methyl (R)-3- (4-amino-5-(3- (5-chloro-3-((1- (tetrahydro-2H- pyran-4-yl) azetidin-3-yl) oxy)thiophene- | 657.2 | E: 1.619 F: 1.049 | (400 MHz, DMSO-d₆) δ ppm 8.07- 7.93 (m, 1H), 7.91-7.82 (m, 1H), 7.24-7.14 (m, 1H), 6.80-6.68 (m, 3H), 4.60-4.47 (m, 4H), 4.35-4.27 (m, 2H), 4.22-4.03 (m, 3H), 3.84- 3.71 (m, 4H), 3.66 (d, J = 4.6 Hz, 3H), 3.20-3.03 (m, 6H), 3.01-2.81 |
| | | | 2-carboxamido) | | | (m, 4H), 1.93-1.65 (m, 5H) |
| | | | piperidin-1-yl) | | | |
| | | | pyrrolo[2,1-f] | | | |
| | | | [1,2,4]triazin-7- | | | |
| | | | yl)-2,5-dihydro- | | | |
| | | | 1H-pyrrole-1- | | | |
| | | | carboxylate | | | |
| 575 | 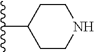 [SMILES: *C1CCNCC1] | 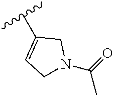 [SMILES: *C1=CCN(C(C)=O)C1] | (R)-N-(1-(7- (1-acetyl-2,5- dihydro-1H- pyrrol-3-yl)-4- aminopyrrolo [2,1-f][1,2,4] | 643.2 | I: 6.944 J: 8.370 | (400 MHz, DMSO-d₆) δ ppm 8.31 (br s, 1H), 7.83 (d, J = 1.5 Hz, 1H), 7.33 (s, 1H), 6.75 (br d, J = 9.0 Hz, 1H), 6.71-6.69 (m, 3H), 4.69-4.53 (m, 2H), 4.46 (br d, J = 5.0 Hz, 2H), 4.26 (br s, 1H), 4.11 (br s, 1H), 3.11 |
| | | | triazin-5-yl) | | | (br s, 1H), 2.94 (br s, 4H), 2.81-2.63 |
| | | | piperidin-3-yl)- | | | (m, 4H), 2.05 (s, 2H), 2.00 (s, 3H), |
| | | | 5-chloro-3- | | | 1.84-1.78 (m, 3H), 1.60 (br s, 3H) |
| | | | (piperidin-4- | | | |
| | | | yloxy)thiophene- | | | |
| | | | 2-carboxamide | | | |
| 576 | 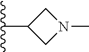 [SMILES: *C1CN(C)C1] | 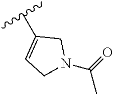 [SMILES: *C1=CCN(C(C)=O)C1] | (R)-N-(1-(7-(1- acetyl-2,5-dihydro- 1H-pyrrol-3-yl)- 4-aminopyrrolo [2,1-f][1,2,4] triazin-5-yl) | 571.2 | E: 1.398 F: 0.932 | (400 MHz, DMSO-d₆) δ ppm 8.31 (br s, 1H), 7.87 (s, 1H), 7.56-7.43 (m, 1H), 7.15 (s, 1H), 6.81-6.72 (m, 3H), 4.98 (br d, J = 5.4 Hz, 1H), 4.73-4.66 (m, 1H), 4.53-4.45 (m, 2H), 4.27 (br s, 1H), 4.21-4.16 |
| | | | piperidin-3-yl)- | | | (m, 1H), 3.72-3.66 (m, 2H), 3.20- |
| | | | 5-chloro-3-((1- | | | 3.08 (m, 3H), 3.00-2.80 (m, 3H), |
| | | | methylazetidin- | | | 2.27 (br d, J = 1.2 Hz, 3H), 2.03 |
| | | | 3-yl)oxy) | | | (d, J = 16,4 Hz, 3H), 1.90-1.57 |
| | | | thiophene-2- | | | (m, 4H) |
| | | | carboxamide | | | |
| 577 | 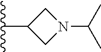 [SMILES: *C1CN(C(C)C)C1] | 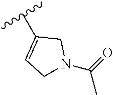 [SMILES: *C1=CCN(C(C)=O)C1] | (R)-N-(1-(7-(1- acetyl-2,5- dihydro-1H- pyrrol-3- yl)-4-amino- pyrrolo[2,1- | 599.2 | E: 1.601 F: 0.984 | (400 MHz, DMSO-d6) δ ppm 8.10 (s, 1H), 7.87 (d, J = 1.7 Hz, 1H), 7.52 (br s, 1H), 7.18 (s, 1H), 6.83- 6.65 (m, 2H), 4.95 (br d, J = 5.4 Hz, 1H), 4.74-4.64 (m, 1H), 4.54-4.40 (m, 2H), 4.27 (br d, J = 2.2 Hz, |
| | | | f][1,2,4]triazin- | | | 1H), 4.23-4.13 (m, 1H), 3.66-3.50 |
| | | | 5-yl)piperidin- | | | (m, 2H), 3.14-2.79 (m, 6H), 2.26- |
| | | | 3-yl)-5-chloro- | | | 2.12 (m, 1H), 2.03 (d, J = 16.6 Hz, |
| | | | 3-((1-isopropyl- | | | 3H), 1.90-1.57 (m, 4H), 0.78 (br d, |
| | | | azetidin-3-yl) | | | J = 2.2 Hz, 6H) |
| | | | oxy)thiophene- | | | |
| | | | 2-carboxamide | | | |
| 578 | 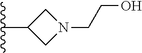 [SMILES: *C1CN(CCO)C1] | 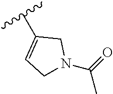 [SMILES: *C1=CCN(C(C)=O)C1] | (R)-N-(1-(7-(1- acetyl-2,5- dihydro-1H- pyrrol-3-yl)- 4-aminopyrrolo [2,1-f][1,2,4] | 601.2 | B: 1.312 F: 0.927 | (400 MHz, DMSO-d₆) δ ppm 8.05 (br s, 1H), 7.86 (s, 1H), 7.59-7.33 (m, 1H), 7.12 (s, 1H), 6.81-6.70 (m, 3H), 5.20-4.92 (m, 1H), 4.69 (br s, 2H), 4.48 (br d, J = 7.1 Hz, 2H), 4.26 (br s, 3H), 3.90-3.82 |
| | | | triazin-5-yl) | | | (m, 3H), 3.68-3.55 (m, 2H), 3.33- |
| | | | piperidin-3-yl)- | | | 3.22 (m, 1H), 3.33-3.20 (m, 1H), |
| | | | 5-chloro-3-((1- | | | 3.17 (br d, J = 4.9 Hz, 2H), 2.99- |
| | | | (2-hydroxyethyl) | | | 2.77 (m, 3H), 2.68 (br s, 1H), |
| | | | azetidin-3-yl) | | | 2.03 (d, J = 16.6 Hz, 3H), 1.83 |
| | | | oxy)thiophene- | | | (br s, 2H) |
| | | | 2-carboxamide | | | |
| 579 | 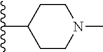 [SMILES: *C1CCN(C)CC1] | 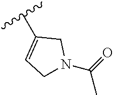 [SMILES: *C1=CCN(C(C)=O)C1] | (R)-N-(1-(7-(1- acetyl-2,5- dihydro-1H- pyrrol-3-yl)-4- aminopyrrolo [2,1-f][1,2,4] | 599.2 | E: 1.421 F: 0.966 | (400 MHz, DMSO-d₆) δ ppm 8.05 (br s, 1H), 7.86 (s, 1H), 7.38 (s, 1H), 6.98-6.87 (m, 1H), 6.82-6.58 (m, 2H), 4.74-4.41 (m, 4H), 4.32-4.08 (m, 2H), 3.71-3.51 (m, 1H), 3.69- 3.50 (m, 6H), 3.34-3.23 (m, 2H), |
| | | | triazin-5-yl) | | | 3.20-3.06 (m, 2H), 3.00-2.88 (m, |
| | | | piperidin-3-yl)- | | | 1H), 2.83-2.70 (m, 1H), 2.40-2.16 |
| | | | 5-chloro-3-((1- | | | (m, 4H), 2.09-2.03 (m, 2H), 1.91- |
| | | | methylpiperidin- | | | 1.73 (m, 4H) |
| | | | 4-yl)oxy) | | | |
| | | | thiophene-2- | | | |
| | | | carboxamide | | | |
| 580 | 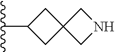 [SMILES: *C1CC2(CNC2)C1] | 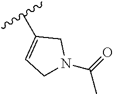 [SMILES: *C1=CCN(C(C)=O)C1] | (R)-3-((2- azaspiro[3.3] heptan-6- yloxy)-N-(1- (7-(1-acetyl- 2,5-dihydro- | 597.2 | E: 1.202 F: 1.020 | (400 MHz, DMSO-d₆) δ ppm 8.55- 8.45 (m, 1H), 7.92-7.86 (m, 1H), 7.15-7.13 (m, 1H), 7.11-7.06 (m, 1H), 6.99-6.94 (m, 1H), 6.84-6.72 (m, 2H), 4.84-4.66 (m, 3H), 4.54- 4.42 (m, 3H), 4.30-4.11 (m, 3H), |
| | | | 1H-pyrrol-3- | | | 4.01-3.87 (m, 5H), 2.53-2.52 (m, |
| | | | yl)-4-amino- | | | 4H), 2.33-2.27 (m, 4H), 2.04 (d, |
| | | | pyrrolo[2,1-f] | | | J = 16.4 Hz, 3H), 1.87-1.77 (m, |
| | | | [1,2,4]triazin- | | | 3H) |
| | | | 5-yl)piperidin- | | | |
| | | | 3-yl)-5-chloro- | | | |
| | | | thiophene-2- | | | |
| | | | carboxamide | | | |
| 581 | 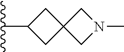 [SMILES: *C1CC2(C1)CN(C)C2] | 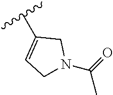 [SMILES: *C1=CCN(C(C)=O)C1] | (R)-N-(1-(7-(1- acetyl-2,5- dihydro-1H- pyrrol-3-yl)-4- aminopyrrolo [2,1-f][1,2,4] | 611.2 | E: 1.245 F: 1.031 | (400 MHz, DMSO-d₆) δ ppm 8.05 (br s, 1H), 7.90 (s, 1H), 7.13 (s, 1H), 7.09 (s, 1H), 6.98-6.90 (m, 1H), 6.84-6.69 (m, 2H), 4.86-4.65 (m, 3H), 4.56-4.39 (m, 3H), 4.31- 4.15 (m, 3H), 4.10-3.91 (m, 3H), |
| | | | triazin-5-yl) | | | 3.18-2.92 (m, 2H), 2.78 (br d, J = |
| | | | piperidin-3-yl)- | | | 4.9 Hz, 5H), 2.55-2.53 (m, 3H), |
| | | | 5-chloro-3-((2- | | | 2.06-2.01 (m, 3H), 1.89-1.76 (m, |
| | | | methyl-2- | | | 3H) |
| | | | azaspiro[3.3] | | | |
| | | | heptan-6-yl) | | | |
| | | | oxy)thiophene- | | | |
| | | | 2-carboxamide | | | |
| 582 | 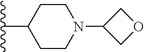 [SMILES: *C1CCN(C2COC2)CC1] | 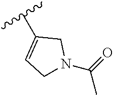 [SMILES: *C1=CCN(C(C)=O)C1] | (R)-N-(1-(7-(1- acetyl-2,5- dihydro-1H- pyrrol-3- yl)-4-amino- pyrrolo[2,1-f] | 641.2 | E: 1.517 F: 0.996 | (400 MHz, DMSO-d₆) δ ppm 8.05 (br s, 1H), 7.90 (s, 1H), 7.48-7.40 (m, 1H), 7.22 (s, 1H), 6.96 (s, 1H), 6.85-6.69 (m, 2H), 4.80-4.61 (m, 6H), 4.55-4.43 (m, 3H), 4.34-4.10 (m, 5H), 3.15-2.78 (m, 8H), 2.53 |
| | | | [1,2,4]triazin- | | | (br s, 2H), 2.06-2.01 (m, 3H), |
| | | | 5-yl)piperidin- | | | 1.91-1.76 (m, 3H) |
| | | | 3-yl)-5-chloro- | | | |
| | | | 3-((1-(oxetan-3- | | | |
| | | | yl)piperidin-4- | | | |
| | | | yloxy)thiophene- | | | |
| | | | 2-carboxamide | | | |
| 583 | 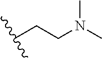 [SMILES: *CCN(C)C] | 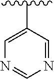 [SMILES: *c1cncnc1] | (R)-N-(1-(4- amino-7- (pyrimidin-5- yl)pyrrolo [2,1-f][1,2,4] | 542.1 | E: 1.581 F: 0.888 | (400 MHz, DMSO-d₆) δ ppm 9.46 (s, 2H), 9.08 (s, 1H), 8.22-8.06 (m, 1H), 7.92 (s, 1H), 7.75-7.57 (m, 1H), 7.35 (s, 1H), 7.30 (s, 1H), 7.05-6.80 (m, 1H), 4.34 (br s, 3H), 4.24-4.09 |
| | | | triazin-5-yl) | | | (m, 2H), 3.68-3.55 (m, 2H), 3.17 |
| | | | piperidin-3- | | | (br d, J = 4.6 Hz, 2H), 2.68 (br s, |
| | | | yl)-5-chloro- | | | 3H), 2.34 (br s, 3H), 2.07 (s, 1H), |
| | | | 3-(2-(dimethyl- | | | 1.95-1.77 (m, 3H) |
| | | | amino)ethoxy) | | | |
| | | | thiophene-2- | | | |
| | | | carboxamide | | | |
| 584 | 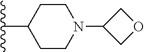 [SMILES: *C1CCN(C2COC2)CC1] | 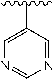 [SMILES: *c1cncnc1] | (R)-N-(1-(4- amino-7- (pyrimidin-5- yl)pyrrolo[2,1- f][1,2,4]triazin- | 610.2 | E: 1.528 F: 0.969 | (400 MHz, DMSO-d6) δ ppm 9.48 (s, 2H), 9.09 (s, 1H), 8.05 (br s, 1H), 7.92 (s, 1H), 7.40 (s, 2H), 7.32 (s, 1H), 4.67-4.60 (m, 1H), 4.46 (br t, J = 6.4 Hz, 1H), 4.39- |
| | | | 5-yl)piperidin- | | | 4.28 (m, 3H), 4.24-4.17 (m, 1H), |
| | | | 3-yl)-5-chloro- | | | 2.98 (br d, J = 1.7 Hz, 5H), 2.38 |
| | | | 3-((1-(oxetan-3- | | | (br s, 1H), 2.08 (s, 2H), 2.00- |
| | | | yl)piperidin-4- | | | 1.53 (m, 8H) |
| | | | yloxy)thiophene- | | | |
| | | | 2-carboxamide | | | |
| 585 | 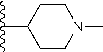 [SMILES: *C1CCN(C)CC1] | 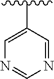 [SMILES: *c1cncnc1] | (R)-N-(1-(4- amino-7- (pyrimidin-5- yl)pyrrolo[2,1- f][1,2,4]triazin- | 568.1 | E: 1.464 F: 1.017 | (400 MHz, DMSO-d6) δ ppm 9.46 (s, 2H), 9.08 (s, 1H), 7.91 (s, 1H), 7.40 (s, 2H), 7.30 (s, 1H), 4.63- 4.56 (m, 1H), 4.22-4.15 (m, 1H), 3.22 (br d, J = 1.3 Hz, 1H), 3.00 |
| | | | 5-yl)piperidin- | | | (br d, J = 3.3 Hz, 1H), 2.86 (br s, |
| | | | 3-yl)-5-chloro- | | | 2H), 2.47-2.41 (m, 1H), 2.13 (s, |
| | | | 3-((1-methyl- | | | 6H), 2.00-1.79 (m, 6H), 1.76-1.63 |
| | | | piperidin-4-yl) | | | (m, 2H) |
| | | | oxy)thiophene- | | | |
| | | | 2-carboxamide | | | |
| 586 | 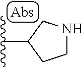 [SMILES: *C1CCNC1] | 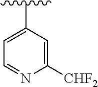 [SMILES: *c1ccnc(C(F)F)c1] | N-((R)-1-(4- amino-7-(2- (difluoromethyl) pyridin-4-yl) pyrrolo[2,1-f] [1,2,4]triazin-5- | 589.2 | E: 1.497 F: 1.117 | (400 MHz, DMSO-d6) δ ppm 8.70 (d, J = 5.4 Hz, 1H), 8.49 (s, 1H), 8.31 (d, J = 3.9 Hz, 1H), 7.99 (s, 1H), 7.58-7.51 (m, 1H), 7.47 (s, 1H), 7.34 (s, 1H), 6.98 (t, J = 55.0 Hz, 1H), 5.10-5.03 (m, 1H), |
| | | | yl)piperidin-3- | | | 4.20-4.11 (m, 1H), 3.08-2.99 |
| | | | yl)-5-chloro-3- | | | (m, 4H), 2.93-2.79 (m, 3H), |
| | | | (pyrrolidin-3- | | | 2.06 (s, 1H), 1.94-1.79 (m, 6H) |
| | | | yloxy)thiophene- | | | |
| | | | 2-carboxamide | | | |
| 587 | 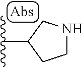 [SMILES: *C1CCNC1] | 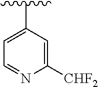 [SMILES: *c1ccnc(C(F)F)c1] | N-((R)-1-(4- amino-7-(2- (difluoromethyl) pyridin-4-yl) pyrrolo[2,1-f [1,2,4]triazin-5- | 589.1 | E: 1.461 F: 1.112 | |
| | | | yl)piperidin-3- | | | |
| | | | yl)-5-chloro-3- | | | |
| | | | (pyrrolidin-3- | | | |
| | | | yloxy)thiophene- | | | |
| | | | 2-carboxamide | | | |
| 588 | 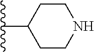 [SMILES: *C1CCNCC1] | 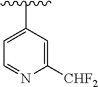 [SMILES: *c1ccnc(C(F)F)c1] | (R)-N-(1-(4- amino-7-(2 (difluoromethyl) pyridin-4-yl) pyrrolo[2,1-f] [1,2,4]triazin-5- | 603.2 | E: 1.475 F: 1.143 | (400 MHz, DMSO-d6) δ ppm 8.69 (d, J = 5.1 Hz, 1H), 8.49 (s, 1H), 8.30 (s, 1H), 7.99 (s, 1H), 7.49 (s, 1H), 7.41 (s, 1H), 6.98 (t, J = 54.3 Hz, 1H), 4.64-4.58 (m, 1H), 4.23-4.14 (m, 1H), 3.05-2.99 |
| | | | yl)piperidin-3- | | | (m, 1H), 2.97-2.89 (m, 3H), |
| | | | yl)-5-chloro-3- | | | 2.85 (br d, J = 5.1 Hz, 1H), |
| | | | (piperidin-4- | | | 2.78-2.76 (m, 1H), 2.65-2.56 |
| | | | yloxy)thiophene- | | | (m, 2H), 2.01-1.80 (m, 7H), |
| | | | 2-carboxamide | | | 1.60-1.45 (m, 3H) |
| 589 | 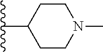 [SMILES: *C1CCN(C)CC1] | 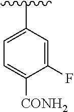 [SMILES: *c1ccc(C(N)=O)c(F)c1] | (R)-N-(1-(4- amino-7-(4- carbamoyl-3- fluorophenyl) pyrrolo[2,1-f] [1,2,4]triazin-5- yl)piperidin-3- | 627.2 | E: 1.487 F: 1.005 | (400 MHz, DMSO-d6) δ ppm 8.12 (dd, J = 1.4, 12.9 Hz, 1H), 8.00 (dd, J = 1.5, 8.3 Hz, 1H), 7.91 (s, 1H), 7.73 (t. J = 8.1 Hz, 1H), 7.68-7.55 (m, 2H), 7.40 (s, 2H), 7.24 (s, 1H), 6.82 (s, 1H), 4.65- 4.52 (m, 1H), 4.19 (br s, 1H), |
| | | | yl)-5-chloro-3- | | | 3.19 (br d, J = 15.5 Hz, 2H), |
| | | | ((1-methyl- | | | 3.07-2.96 (m, 1H), 2.86 (br s, |
| | | | piperidin-4-yl) | | | 2H), 2.36-2.26 (m, 2H), 2.22- |
| | | | oxy)thiophene- | | | 2.11 (m, 3H), 2.04-1.63 (m, 8H) |
| | | | 2-carboxamide | | | |
| 590 | 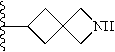 [SMILES: *C1CC2(CNC2)C1] | 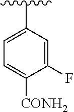 [SMILES: *c1ccc(C(N)=O)c(F)c1] | (R)-3-((2- azaspiro[3.3] heptan-6-yl) oxy)-N-(1-(4- amino-7-(4- carbamoyl-3- fluorophenyl) | 625.2 | E: 1.207 F: 0.993 | (400 MHz, DMSO-d6) δ ppm 8.14 (dd, J = 1.5, 13.2 Hz, 1H), 8.02 (dd, J = 1.5, 8.3 Hz, 1H), 7.93 (s, 1H), 7.75 (t, J = 8.2 Hz, 1H), 7.70- 7.60 (m, 2H), 7.28 (s, 1H), 7.17 (s, 1H), 4.78 (br d, J = 7.1 Hz, 1H), 4.20 (br s, 1H), 3.61 (s, 2H), 3.51- |
| | | | pyrrolo[2,1-f] | | | 3.50 (m, 2H), 3.15 (br s, 1H), |
| | | | [1,2,4]triazin-5- | | | 3.05-2.86 (m, 4H), 2.75-2.68 (m, |
| | | | yl)piperidin-3- | | | 2H), 2.26-2.17 (m, 2H), 1.91-1.76 |
| | | | yl)-5-chloro- | | | (m, 5H), 1.63 (br s, 2H) |
| | | | thiophene-2- | | | |
| | | | carboxamide | | | |
| 591 | 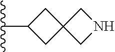 [SMILES: *C1CC2(CNC2)C1] | 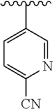 [SMILES: *c1ccc(C#N)nc1] | (R)-3-((2- azaspiro[3.3] heptan-6-yloxy)- N-(1-(4-amino- 7-(6-cyano- pyridin-3- yl)pyrrolo[2,1- | 590.2 | E: 1.363 F: 1.149 | (400 MHz, DMSO-d₆) δ ppm 9.52- 9.34 (m, 2H), 8.86-8.80 (m, 1H), 7.97 (s, 1H), 7.44 (s, 1H), 7.16 (s, 1H), 4.91-4.68 (m, 3H), 4.26-4.09 (m, 4H), 3.03-2.82 (m, 4H), 2.68 (br s, 3H), 1.84 (br s, 4H), 1.68- 1.49 (m, 2H) |
| | | | f][1,2,4]triazin- | | | |
| | | | 5-yl)piperidin- | | | |
| | | | 3-yl)-5-chloro- | | | |
| | | | thiophene-2- | | | |
| | | | carboxamide | | | |
| 592 | 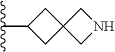 [SMILES: *C1CC2(CNC2)C1] | 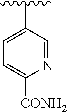 [SMILES: *c1ccc(C(N)=O)nc1] | (R)-5-(5-(3-(3- ((2-azaspiro[3.3] heptan-6-yl) oxy)-5-chloro- thiophene-2- carboxamido) piperidin-1- | 608.2 | E: 1.149 F: 0.953 | (400 MHz, DMSO-d6) δ ppm 9.31 (d, J = 1.7 Hz, 1H), 8.69 (dd, J = 2.1, 8.2 Hz, 1H), 8.10 (d, J = 8.3 Hz, 3H), 7.93 (s, 1H), 7.65 (br d, J = 1.5 Hz, 1H), 7.33 (s, 1H), 7.16 (s, 1H), 4.77 (br t, J = 6.8 Hz, 1H), 4.22-4.15 (m, 1H), |
| | | | yl)-4-amino- | | | 3.70 (br s, 2H), 3.61-3.58 (m, |
| | | | pyrrolo[2,1-f] | | | 2H), 3.04-2.87 (m, 5H), 2.73 |
| | | | [1,2,4]triazin-7- | | | (br dd, J = 1.0, 7.3 Hz, 2H), |
| | | | yl)picolinamide | | | 2.30-2.21 (m, 2H), 1.95-1.76 |
| | | | | | | (m, 5H), 1.64 (br s, 1H) |
| 593 | 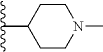 [SMILES: *C1CCN(C)CC1] | 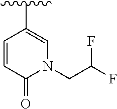 [SMILES: *c1ccc(=O)n(CC(F)F)c1] | (R)-N-(1-(4- amino-7-(1- (2,2-difluoro- ethyl)-6-oxo- 1,6-dihydro- pyridin-3-yl) pyrrolo[2,1-f] | 647.2 | E: 1.570 F: 1.058 | (400 MHz, DMSO-d6) δ ppm 8.49 (d, J = 2.5 Hz, 1H), 8.10 (dd, J = 2.5, 9.5 Hz, 1H), 7.82 (s, 1H), 7.40 (s, 2H), 6.91 (s, 1H), 6.57 (d, J = 9.5 Hz, 2H), 6.33 (tt, J = 4.0, 56.5 Hz, 1H), 4.65-4.53 (m, 1H), 4.45 (dt, J = 3.9, 14.7 Hz, |
| | | | [1,2,4]triazin- | | | 2H), 4.24-4.10 (m, 2H), 3.21-3.13 |
| | | | 5-yl)piperidin- | | | (m, 1H), 3.03-2.95 (m, 1H), 2.82 |
| | | | 3-yl)-5-chloro- | | | (br s, 3H), 2.60-2.54 (m, 1H), |
| | | | 3-((1-methyl- | | | 2.30-2.25 (m, 1H), 2.17 (br s, |
| | | | piperidin-4-yl) | | | 3H), 2.02-1.78 (m, 5H), 1.78- |
| | | | oxy)thiophene- | | | 1.64 (m, 2H) |
| | | | 2-carboxamide | | | |
| 594 | 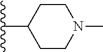 [SMILES: *C1CCN(C)CC1] | 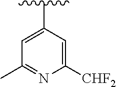 [SMILES: *c1cc(C)nc(C(F)F)c1] | (R)-N-(1-(4- amino-7-(4- carbamoyl-3- fluorophenyl) pyrrolo[2,1-f] [1,2,4]triazin-5- | 627.2 | E: 1.487 F: 1.005 | (400 MHz, DMSO-d6) δ ppm 8.12 (dd, J = 1.4, 12.9 Hz, 1H), 8.00 (dd, J = 1.5, 8.3 Hz, 1H), 7.91 (s, 1H), 7.73 (t, J = 8.1 Hz, 1H), 7.68-7.55 (m, 2H), 7.40 (s, 2H), 7.24 (s, 1H), 6.82 (s, 1H), 4.65-4.52 |
| | | | yl)piperidin-3- | | | (m, 1H), 4.19 (br s, 1H), 3.19 (br d, |
| | | | yl)-5-chloro- | | | J = 15,5 Hz, 2H), 3.07-2.96 (m, 1H), |
| | | | 3-((1-methyl- | | | 2.86 (s, 3H), 2.36-2.26 (m, 2H), |
| | | | piperidin-4-yl) | | | 2.22-2.11 (m, 3H), 2.04-1.63 (m, |
| | | | oxy)thiophene- | | | 8H) |
| | | | 2-carboxamide | | | |

### Example 595

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpiperidin-3-yl)oxy)thiophene-2-carboxamide

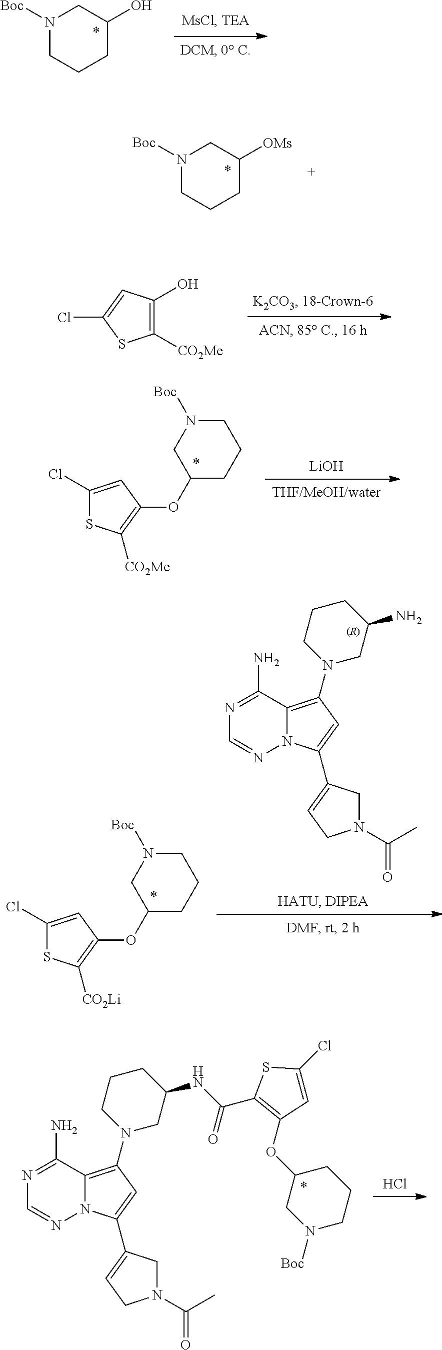

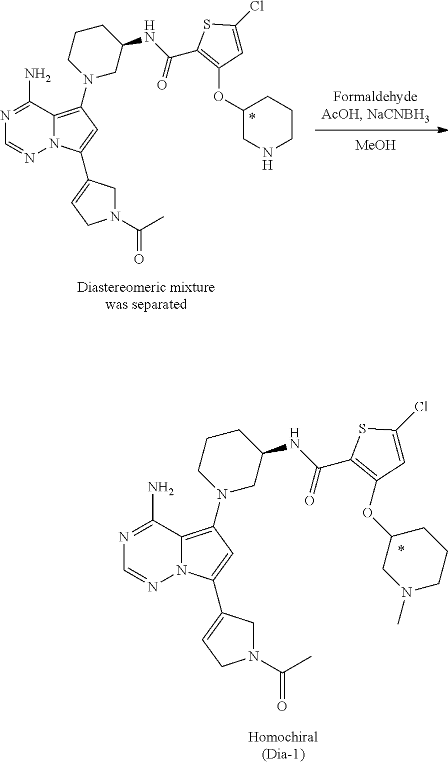 [SMILES: "MeOH" ; CC(=O)N1CC=C(c2cc(N3CCC[C@@H](NC(=O)c4sc(Cl)cc4OC4CCCNC4)C3)c3c(N)ncnn23)C1 ; "FormaldehydeAcOH, NaCNBH3" ; CC(=O)N1CC=C(c2cc(N3CCC[C@@H](NC(=O)c4sc(Cl)cc4OC4CCCN(C)C4)C3)c3c(N)ncnn23)C1]

### Example 595A. tert-butyl 3-((methylsulfonyl)oxy)piperidine-1-carboxylate

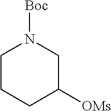 [SMILES: CC(C)(C)OC(=O)N1CCCC(OS(C)(=O)=O)C1]

To a solution of tert-butyl 3-hydroxypiperidine-1-carboxylate (2.5 g, 12.4 mmol) in DCM (25 mL), were added TEA (3.46 mL, 24.8 mmol) and MsCl (1.16 mL, 14.9 mmol) at 0° C. and stirred at rt for 3 h. The reaction was diluted with DCM and washed with Sat. NaHCO₃ solution and brine. Organic layer was separated, dried over Na₂SO₄, filtered and concentrated to afford Example 595A (3.3 g, 95% yield) as a yellow solid; ¹H NMR (400 MHz, CDCl₃) δ ppm 4.72 (br s, 1H), 3.71-3.56 (m, 2H), 3.50-3.27 (m, 2H), 3.06 (s, 3H), 2.05-1.73 (m, 3H), 1.56 (br d, J=7.0 Hz, 1H), 1.47 (s, 9H).

### Example 595B. tert-butyl 3-((5-chloro-2-(methoxycarbonyl)thiophen-3-yl)oxy)piperidine-1-carboxylate

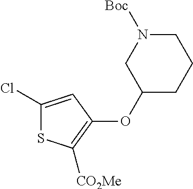 [SMILES: COC(=O)c1sc(Cl)cc1OC1CCCN(C(=O)OC(C)(C)C)C1]

Analogous to the procedure for the preparation of Example 346A, reaction of methyl 5-chloro-3-hydroxythiophene-2-carboxylate (300 mg, 1.56 mmol) and Example 595A (1.09 g, 3.89 mmol) afforded Example 595B (390 mg, 67% yield) as a yellow thick liquid. MS: [M+H-t-Bu]⁺=320.0; ¹H NMR (400 MHz, CDCl₃) δ ppm 6.80 (s, 1H), 4.25-4.19 (m, 1H), 3.94-3.71 (m, 4H), 3.57 (br s, 1H), 3.29 (br s, 2H), 2.07 (s, 1H), 1.90 (br s, 2H), 1.51 (s, 1H), 1.48-1.41 (m, 9H).

### Example 595C. 3-((1-(tert-butoxycarbonyl)piperidin-3-yl)oxy)-5-chlorothiophene-2-carboxylic acid

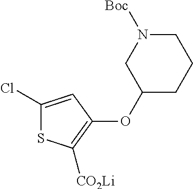

Analogous to the procedure for the preparation of Example 346B, reaction of Example 595B (390 mg, 1.04 mmol) and lithium hydroxide (87 mg, 2.08 mmol) afforded Example 595C (330 mg, 88% yield) as a white solid. MS: [M+H-t-Bu]⁺=306.0; ¹H NMR (400 MHz, DMSO-d₆) δ ppm 6.71 (br s, 1H), 4.81-4.36 (m, 1H), 3.65 (br s, 2H), 3.47 (br s, 2H), 3.24-3.00 (m, 1H), 1.96-1.62 (m, 3H), 1.37 (br s, 9H).

### Example 595D. tert-butyl 3-((2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)piperidine-1-carboxylate

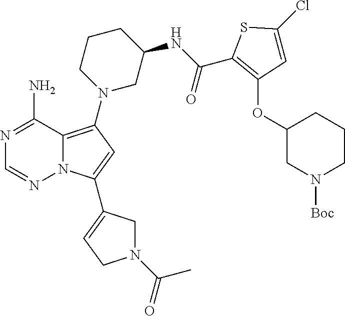 [SMILES: CC(=O)N1CC=C(c2cc(N3CCC[C@@H](NC(=O)c4sc(Cl)cc4OC4CCCN(C(=O)OC(C)(C)C)C4)C3)c3c(N)ncnn23)C1]

Analogous to the procedure for the preparation of Example 561C, reaction of Intermediate 32 and Example 595C (400 mg, 1.09 mmol) afforded Example 595D (280 mg, 38% yield) as a yellow solid. MS: [M+H-t-Bu]⁺=685.2; ¹H NMR (400 MHz, DMSO-d₆) δ ppm 8.08-7.82 (m, 2H), 7.49-7.37 (m, 1H), 7.24-7.10 (m, 1H), 6.83-6.61 (m, 3H), 4.83-4.60 (m, 2H), 4.54-4.39 (m, 3H), 4.34-4.20 (m, 1H), 4.15-3.85 (m, 2H), 3.73-3.54 (m, 1H), 3.27-3.09 (m, 2H), 3.07-2.96 (m, 1H), 2.92-2.79 (m, 2H), 2.09-1.97 (m, 4H), 1.90-1.51 (m, 7H), 1.43-1.33 (m, 9H).

### Example 595E. N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-(piperidin-3-yloxy)thiophene-2-carboxamide

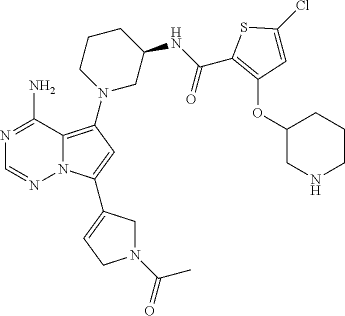 [SMILES: CC(=O)N1CC=C(c2cc(N3CCC[C@@H](NC(=O)c4sc(Cl)cc4OC4CCCNC4)C3)c3c(N)ncnn23)C1]

Analogous to the procedure for the preparation of Example 561, reaction of Example 595D (280 mg, 0.409 mmol) and 4N HCl in dioxane (2.0 mL) afforded Example 595E (150 mg, 61% yield) as a pale yellow solid. LCMS Method I: RT=6.609 min; Method J: RT=8.524 min; MS: [M+H]⁺=585.2; ¹H NMR (400 MHz, DMSO-d₆) δ ppm 8.08-7.86 (m, 2H), 7.83 (s, 1H), 7.25 (s, 1H), 6.81-6.67 (m, 3H), 4.72-4.64 (m, 1H), 4.52-4.41 (m, 3H), 4.31-4.23 (m, 1H), 4.13-4.03 (m, 1H), 3.18-3.06 (m, 1H), 2.98-2.81 (m, 3H), 2.72-2.60 (m, 5H), 2.03 (d, J=17.1 Hz, 3H), 1.91-1.75 (m, 4H), 1.69-1.35 (m, 4H).

Diastereomeric mixture of Example 595E was separated by chiral SFC [Preparative SFC Conditions: Column: Chiralcel-ODH (250*4.6 mm), 5μ, Co-solvent: 0.2% DEA in IPA]

Diastereomer-1: (60 mg, 24% yield) as a pale yellow solid. HP-LC Method I: RT=6.969 min; Method J: RT=8.352 min; MS:[M+H]⁺=585.2; 100% de (Chiral Purity); ¹H NMR (400 MHz, DMSO-d₆) δ ppm 8.37 (br s, 1H), 7.96 (br s, 1H), 7.87 (s, 1H), 7.35 (d, J=1.0 Hz, 1H), 6.85-6.66 (m, 3H), 4.68 (br s, 1H), 4.55 (br s, 1H), 4.48 (br d, J=5.0 Hz, 2H), 4.26 (br s, 1H), 4.12 (br s, 1H), 3.15 (br s, 1H), 2.98 (br s, 1H), 2.79 (br s, 2H), 2.74 (s, 1H), 2.71-2.58 (m, 3H), 2.57-2.52 (m, 2H), 2.06-1.86 (m, 4H), 1.86-1.73 (m, 4H), 1.69 (br s, 1H), 1.47 (br s, 1H)

Diastereomer-2: (450 mg, 17% yield) as a pale yellow solid. HP-LC Method I: RT=6.714 min; Method K: RT=13.662 min; MS:[M+H]⁺=585.2; 95% de (Chiral Purity); ¹H NMR (400 MHz, DMSO-d₆) δ ppm 8.29-8.16 (m, 1H), 8.08-7.94 (m, 1H), 7.87 (d, J=1.0 Hz, 1H), 7.35 (d, J=1.0 Hz, 1H), 6.81-6.69 (m, 3H), 4.69 (br d, J=2.0 Hz, 1H), 4.48 (br d, J=6.5 Hz, 3H), 4.30-4.23 (m, 1H), 4.17-4.04 (m, 1H), 3.23-3.13 (m, 1H), 3.03-2.95 (m, 1H), 2.85-2.59 (m, 6H), 2.03 (d, J=16.1 Hz, 3H), 1.87-1.60 (m, 5H), 1.55-1.38 (m, 2H), 1.29-1.20 (m, 1H), 1.18-1.06 (m, 1H).

### Example 595

### N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-methylpiperidin-3-yl)oxy)thiophene-2-carboxamide

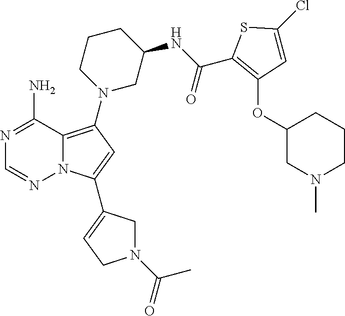 [SMILES: CC(=O)N1CC=C(c2cc(N3CCC[C@@H](NC(=O)c4sc(Cl)cc4OC4CCCN(C)C4)C3)c3c(N)ncnn23)C1]

Analogous to the procedure for the preparation of Example 375, reaction of Example 595E (Dia-1: 30 mg, 0.051 mmol) and formaldehyde solution (aq. 38%, 0.020 mL, 0.26 mmol) afforded Example 595 (Dia-1: 23.6 mg, 77% yield) as a pale yellow solid. LC-MS Method E: RT=1.751 min, [M+H]⁺=599.2; LC-MS Method F: RT=1.002 min, [M+H]⁺=599.3; ¹H NMR (400 MHz, DMSO-d₆) δ ppm 8.29-8.16 (m, 1H), 7.91-7.85 (m, 1H), 7.40-7.35 (m, 1H), 6.80-6.67 (m, 3H), 4.72 (br s, 2H), 4.52-4.41 (m, 2H), 4.28 (br s, 1H), 4.17 (br s, 2H), 3.25-3.13 (m, 3H), 3.09-2.86 (m, 5H), 2.29-2.19 (m, 2H), 2.03 (d, J=15.4 Hz, 3H), 1.96-1.66 (m, 5H), 1.63-1.39 (m, 2H), 1.29-1.18 (m, 1H).

The following examples in Table 43 were prepared using the same procedures as shown in Example 596 (diastereomer-1).

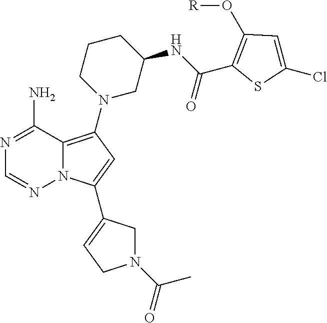

TABLE 43

| | | | | HPLC | |
|---|---|---|---|---|---|
| | | | LCMS | Method, | |
| Example | R | Name | (M + H) | RT (min.) | ¹H NMR |
| 597 (Diasteromer-2) | 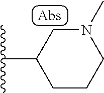 [SMILES: *C1CCCN(C)C1] | N-((R)-1-(7-(1-acetyl- 2,5-dihydro-1H- pyrrol-3-yl)-4- aminopyrrolo[2,1- f\|[1,2,4]triazin-5- yl)piperidin-3-y1)-5- | 599.2 | E: 1.713 F: 0.994 | (400 MHz, DMSO-d₆) δ ppm 8.29-8.16 (m, 1H), 7.87 (s, 1H), 7.45-7.34 (m, 1H), 6.81-6.66 (m, 3H), 4.73-4.63 (m, 2H), 4.54-4.42 (m, 3H), 4.31-4.23 (m, 1H), 4.17-4.04 (m, 1H), 3.24-3.08 (m, 1H), 3.23-3.07 (m, 1H), 3.06-2.89 (m, 3H), 2.84-2.72 (m, 2H), 2.53-2.53 (m, 1H), 2.53-2.52 (m, 1H), 2.24-2.10 (m, 2H), 2.03 (d. J = 15.7 Hz, 4H), 1.96- |
| | | chloro-3-((1- | | | 1.88 (m, 2H), 1.94-1.87 (m, 1H), 1.87-1.77 (m, 3H) |
| | | methylpiperidin-3- | | | |
| | | yl)oxy)thiophene-2- | | | |
| | | carboxamide | | | |
| 598 (Diastereomer-1) | 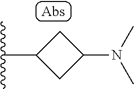 [SMILES: *C1CC(N(C)C)C1] | (R)-N-(1-(7-(1-acetyl- 2,5-dihydro-1H- pyrrol-3-yl)-4- aminopyrrolo[2,1- f][1,2,4]triazin-5- y1)piperidin-3-y1)-5- | 599.2 | I: 4.164 J: 4.291 | (400 MHz, DMSO-d₆) δ ppm 8.12-7.95 (m, 1H), 7.86 (d, J = 1.8 Hz, 1H), 7.64-7.39 (m, 1H), 7.13 (s, 1H), 6.75 (s, 2H), 5.00-4.87 (m, 1H), 4.76-4.62 (m, 1H), 4.56-4.41 (m, 2H), 4.33-4.10 (m, 2H), 3.11-3.02 (m, 2H), 2.99-2.86 (m, 4H), 2.83-2.73 (m, 2H), 2.23-2.12 (m, 2H), 2.03 (d, J = 16.4 Hz, 3H), 1.95 (br s, 7H), 1.85-1.75 (m, 3H), 1.67- |
| | | chloro-3-(3- | | | 1.56 (m, 1H) |
| | | (dimethylamino) | | | |
| | | cyclobutoxy) | | | |
| | | thiophenc-2- | | | |
| | | carboxamide | | | |
| 599 (Diasteromer-2) | 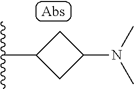 [SMILES: *C1CC(N(C)C)C1] | (R)-N-(1-(7-(1-acetyl- 2,5-dihydro-1H- pyrrol-3-yl)-4- aminopyrrolo[2,1- f][1,2,4]triazin-5- yl)piperidin-3-y1)-5- | 599.2 | 1: 4.078 J: 4.311 | (400 MHz, DMSO-d₆) δ ppm 7.87 (s, 1H), 7.57-7.33 (m, 2H), 7.18 (s, 1H), 6.75 (s, 2H), 4.76-4.61 (m, 2H), 4.53- 4.41 (m, 2H), 4.31-4.12 (m, 2H), 3.15-3.05 (m, 2H), 2.90 (br s, 2H), 2.67 (br d, J = 1.8 Hz, 3H), 2.36-2.28 (m, 2H), 1.98 (m, 8H), 1.88-1.69 (m, 3H), 1.65-1.57 (m, 1H), 1.46-1.18 (m, 1H) |
| | | chloro-3-(3- | | | |
| | | (dimethylamino)cycl | | | |
| | | obutoxy)thiophene-2- | | | |
| | | carboxamide | | | |

### Example 600

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((3-fluoro-1-methylpiperidin-4-yl)oxy)thiophene-2-carboxamide

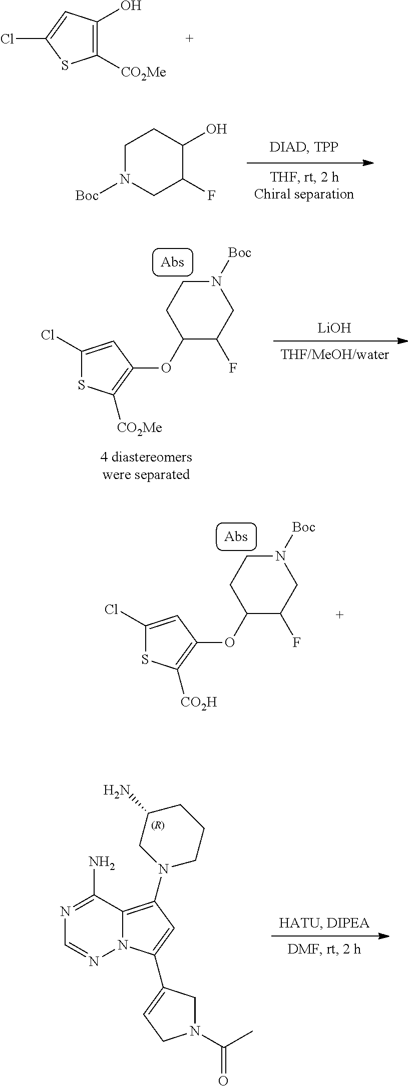

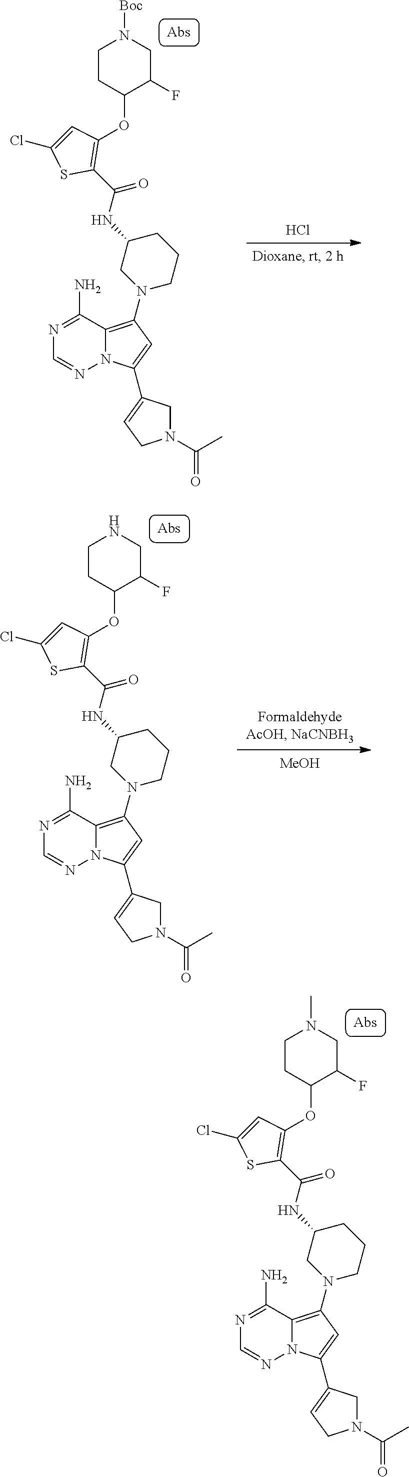

### Example 600A. tert-butyl 4-((5-chloro-2-(methoxycarbonyl)thiophen-3-yl)oxy)-3-fluoropiperidine-1-carboxylate

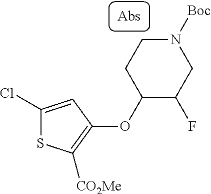 [SMILES: COC(=O)c1sc(Cl)cc1OC1CCN(C(=O)OC(C)(C)C)CC1F]

Analogous to the procedure for the preparation of Example 356A, reaction of methyl 5-chloro-3-hydroxythiophene-2-carboxylate (1.0 g, 5.19 mmol) and tert-butyl 3-fluoro-4-hydroxypiperidine-1-carboxylate (1.25 g, 5.71 mmol) afforded Example 600A (mixture of 4 isomers) as a yellow thick liquid which was separated by SFC to get two sets of diastereomeric mixtures.

Diastereomeric mixture-1 was separated by chiral SFC [Preparative SFC Conditions: Column/dimensions: Chiralpak IC-2(250×4.6) mm, 5 μm; % CO₂: 55% % Co solvent: 15% OF 5 mM 0.2% DEA in IPA, Total Flow: 4 g/min; Back Pressure: 100bar; Temperature: 35° C.; UV: 280 nm] to afford Dia-1 (280 mg, 13.7% yield) as a white solid, MS: [M+H-t-Bu]⁺=338.0, 100% de; ¹H NMR (400 MHz, CDCl₃) δ ppm 6.78 (s, 1H), 4.74-4.40 (m, 2H), 3.84 (s, 5H), 3.61-3.37 (m, 2H), 2.22-2.06 (m, 1H), 1.92-1.76 (m, 1H), 1.49 (s, 9H); ¹⁹F NMR (400 MHz, CDCl₃) δ ppm −191.501 and Dia-2 (270 mg, 13.2% yield) as a white solid, MS: [M+H-t-Bu]⁺=338.0; 100% de; ¹H NMR (400 MHz, CDCl₃) δ ppm 6.78 (s, 1H), 4.75-4.42 (m, 2H), 3.94-3.68 (m, 5H), 3.63-3.41 (m, 2H), 2.21-2.07 (m, 1H), 1.91-1.77 (m, 1H), 1.49 (s, 9H); ¹⁹F NMR (400 MHz, CDCl₃) δ ppm −191.502.

Diastereomeric mixture-2 was separated by chiral SFC [Chiralpak IG-2(250×4.6 mm), 5 μm;% CO₂: 55%, % Co solvent: 15% OF 5 mM 0.2% DEA in IPA; Total Flow: 4 g/min; Back Pressure: 100 bar; Temperature: 25° C.; UV: 280 nm] to afford Dia-3 (240 mg, 11.74% yield) as a white solid, MS: [M+H-t-Bu]⁺=338.0; 100% de; ¹H NMR (400 MHz, CDCl₃) δ ppm 6.77 (s, 1H), 4.93-4.58 (m, 2H), 3.91-3.77 (m, 5H), 3.67-3.46 (m, 2H), 2.18-2.04 (m, 1H), 1.93-1.76 (m, 1H), 1.49 (s, 9H); ¹⁹F NMR (400 MHz, CDCl₃) δ ppm −195.921 and Dia-4 (180 mg, 8.8% yield) as a white solid, MS: [M+H-t-Bu]⁺=338.0; 97.22% de; ¹H NMR (400 MHz, CDCl₃) δ ppm 6.77 (s, 1H), 4.78-4.56 (m, 2H), 3.92-3.72 (m, 5H), 3.66-3.45 (m, 2H), 2.17-2.05 (m, 1H), 1.90-1.75 (m, 1H), 1.49 (s, 9H); ¹⁹F NMR (400 MHz, CDCl₃) δ ppm −195.964.

### Example 600B. 3-((1-(tert-butoxycarbonyl)-3-fluoropiperidin-4-yl)oxy)-5-chlorothiophene-2-carboxylic acid (Dia-1)

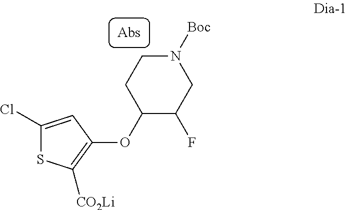

Analogous to the procedure for the preparation of Example 356B, reaction of Example 600A (270 mg, 0.686 mmol) and lithium hydroxide (57.6 mg, 1.37 mmol) afforded as Example 600B as a white solid. MS: [M+H-t-Bu]⁺=324.0; ¹H NMR (400 MHz, DMSO-d₆) δ ppm 6.88 (s, 1H), 4.83-4.53 (m, 2H), 3.76-3.39 (m, 4H), 1.89-1.58 (m, 2H), 1.41 (s, 9H). ¹⁹F NMR (400 MHz, CDCl₃) δ ppm −191.089.

### Example 600C. tert-butyl 4-((2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)-3-fluoropiperidine-1-carboxylate (Dia-1)

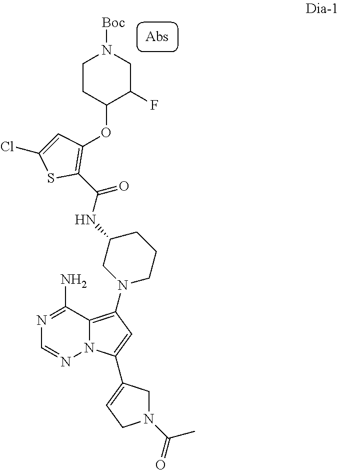 [SMILES: CC(=O)N1CC=C(c2cc(N3CCC[C@@H](NC(=O)c4sc(Cl)cc4OC4CCN(C(=O)OC(C)(C)C)CC4F)C3)c3c(N)ncnn23)C1]

Analogous to the procedure for the preparation of Example 356C, reaction of Example 600B (100 mg, 0.259 mmol) and (R)-1-(3-(4-amino-5-(3-aminopiperidin-1-yl)pyrrolo[2,1-f][1,2,4]triazin-7-yl)-2,5-dihydro-1H-pyrrol-1-yl)ethan-1-one.HCl (98 mg, 0.259 mmol) afforded Example 600C (150 mg, 82% yield) as a yellow solid. MS: [M+H]⁺=703.3; ¹H NMR (400 MHz, DMSO-d₆) δ ppm 8.06-7.91 (m, 1H), 7.86 (d, J=2.0 Hz, 1H), 7.43 (s, 1H), 7.28 (br d, J=8.5 Hz, 1H), 6.81-6.67 (m, 3H), 4.79 (br s, 1H), 4.69 (br s, 2H), 4.48 (br d, J=8.5 Hz, 2H), 4.27 (br s, 1H), 4.15 (br s, 1H), 4.04-3.83 (m, 1H), 3.62 (br s, 1H), 3.30-3.06 (m, 3H), 2.97 (br s, 2H), 2.86-2.69 (m, 1H), 2.15-1.94 (m, 4H), 1.80 (br s, 3H), 1.67 (br s, 1H), 1.56 (br s, 1H), 1.43-1.29 (m, 9H).

### Example 600D

### N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((3-fluoropiperidin-4-yl)oxy)thiophene-2-carboxamide (Dia-1)

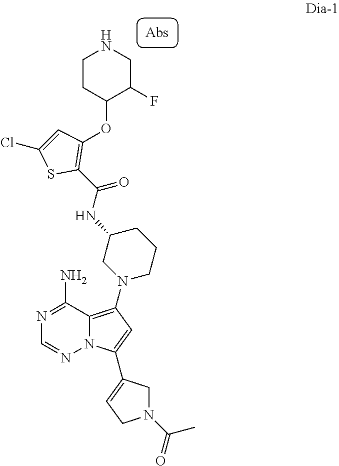 [SMILES: CC(=O)N1CC=C(c2cc(N3CCC[C@@H](NC(=O)c4sc(Cl)cc4OC4CCNCC4F)C3)c3c(N)ncnn23)C1]

Analogous to the procedure for the preparation of Example 561C, reaction of Example 600C (150 mg, 0.213 mmol) and 4N HCl in dioxane (1.07 mL, 4.27 mmol) afforded Example 600D (21.5 mg, 17% yield) as a pale yellow solid. LC-MS Method E: RT=1.499 min, [M+H]⁺=603.2; LC-MS Method F: RT=1.057 min, [M+H]⁺=603.3; ¹H NMR (400 MHz, DMSO-d₆) δ ppm 8.08-7.92 (m, 1H), 7.87 (d, J=1.8 Hz, 1H), 7.40 (s, 2H), 6.87-6.63 (m, 3H), 4.69 (br s, 1H), 4.63 (br s, 2H), 4.48 (br d, J=7.8 Hz, 3H), 4.27 (br s, 1H), 4.16 (br s, 1H), 3.16-3.08 (m, 1H), 2.96 (br s, 2H), 2.90-2.69 (m, 3H), 2.57 (br s, 1H), 2.20-2.11 (m, 1H), 2.09-1.97 (m, 4H), 1.82 (br s, 3H), 1.53 (br d, J=7.3 Hz, 2H); ¹⁹F NMR (400 MHz, CDCl₃) δ ppm −187.096.

### Example 600

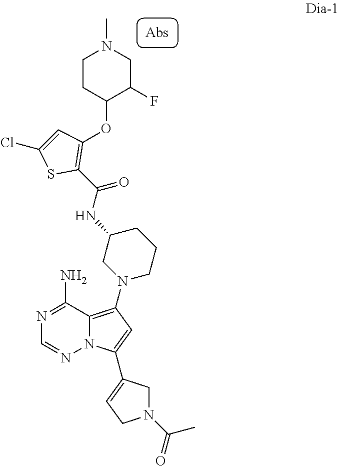 [SMILES: CC(=O)N1CC=C(c2cc(N3CCC[C@@H](NC(=O)c4sc(Cl)cc4OC4CCN(C)CC4F)C3)c3c(N)ncnn23)C1]

Analogous to the procedure for the preparation of Example 375, reaction of Example 600D (70 mg, 0.109 mmol) and formaldehyde solution (aq. 38%, 0.043 mL, 0.55 mmol) afforded Example 600 (20.9 mg, 31% yield) as a pale yellow solid. LC-MS Method E: RT=1.670 min, [M+H]⁺=617.2; LC-MS Method F: RT=1.010 min, [M+H]⁺=617.3; ¹H NMR (400 MHz, DMSO-d₆) δ ppm 8.08-7.92 (m, 1H), 7.87 (d, J=1.5 Hz, 1H), 7.41 (s, 1H), 6.80-6.70 (m, 3H), 4.75-4.40 (m, 5H), 4.30-4.21 (m, 1H), 3.20-3.14 (m, 2H), 3.05-2.75 (m, 6H), 2.26-2.14 (m, 5H), 2.06-1.99 (m, 3H), 1.89-1.64 (m, 5H); ¹⁹F NMR (400 MHz, CDCl₃) δ ppm −188.074.

The following examples in Table 44 were prepared using the same procedure as shown in Example 600, using the appropriate amino alcohol, followed by optional reductive amination.

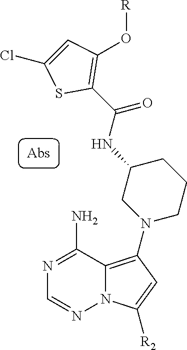

TABLE 44

| | | | | | HPLC | |
|---|---|---|---|---|---|---|
| | | | | | Method, | |
| | | | | LCMS | RT | |
| Example | R | R₂ | Name | (M + H)⁺ | (min.) | ¹H NMR |
| 601 | 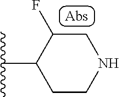 [SMILES: *C1CCNCC1F] | 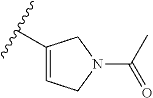 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- yl)piperidin-3-y1)-5- chloro-3-((3- | 603.2 | E: 1.443 F: 0.994 | (400 MHz, DMSO-d₆) δ ppm 8.08-7.90 (m, 1H), 7.87 (d, J = 1.8 Hz, 1H)7.91 (s, 1H), 7.40 (s, 1H), 6.84-6.66 (m, 3H), 5.18-4.82 (m, 2H), 4.74- 4.06 (m, 5H), 3.23-2.97 (m, 8H), 2.81-2.67 (m, 2H), 2.03 (d, J = 15.0 Hz, 3H), 1.94-1.67 (m, 4H), 1.59-1.45 (m, 1H); 19F NMR (400 MHz, |
| | | | fluoropiperidin-4- | | | CDCl₃) δ ppm −191.425 |
| | | | yl)oxy)thiophene-2- | | | |
| | | | carboxamide, | | | |
| | | | Diastereomer-2 | | | |
| 602 | 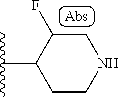 [SMILES: *C1CCNCC1F] | 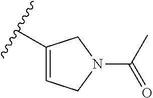 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- y1)piperidin-3-y1)-5- chloro-3-((3- | 603.2 | E: 1.353 F: 0.957 | (400 MHz, DMSO-d₆) δ ppm 9.17 (br s, 1H), 8.74 (br s, 1H), 8.30 (s, 1H), 7.91 (s, 1H), 7.43 (s, 1H), 6.82-6.65 (m, 2H), 5.37-5.19 (m, 1H), 4.94- 4.80 (m, 1H), 4.68 (br d, J = 2.0 Hz, 1H), 4.48 (br d, J = 5.3 Hz, 2H), 4.27 (br d, J = 2.8 Hz, 1H), 4.22-4.13 (m, 1H), 3.75-3.68 (m, 1H), 3.36 |
| | | | fluoropiperidin-4- | | | (br s, 2H), 3.29-3.00 (m, 4H), 2.95 (br s, 1H), |
| | | | yl)oxy)thiophone-2- | | | 2.82 (br s, 2H), 2.29-2.20 (m, 1H), 2.03 (d, J = |
| | | | carboxamide, | | | 15.8 Hz, 4H), 1.94-1.72 (m,3H) |
| | | | Diastereomer-3 | | | |
| 603 | 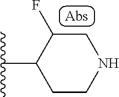 [SMILES: *C1CCNCC1F] | 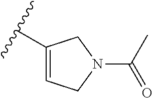 [SMILES: *C1=CCN(C(C)=O)C1] | N-((R)-1-(7-(1-acetyl-2,5- dihydro-1H-pyrrol-3-y1)- 4-aminopyrrolo[2,1- f][1,2,4]triazin-5- yl)piperidin-3-y1)-5- chloro-3-((3- | 603.2 | E: 1.387 F: 0.988 | (400 MHz, DMSO-d₆) δ ppm 8.74 (br s, 1H),7.87 (d, J = 1.8 Hz, 1H), 7.44 (s, 2H), 6.82-6.69 (m, 3H), 5.01-4.63 (m, 3H), 4.48 (br d, J = 6.5 Hz. 2H), 4.30-4.09 (m, 2H), 3.20-3.09 (m, 2H), 2.97 (br s, 3H), 2.89-2.72 (m, 2H), 2.64 (br s, 2H), 2.09-1.96 (m, 4H), 1.96-1.86 (m, 2H), 1.83 (br s, |
| | | | fluoropiperidin-4- | | | 3H). |
| | | | yl)oxy)thiophene~2- | | | |
| | | | carboxamide, | | | |
| | | | Diastereomer-4 | | | |
| 604 | 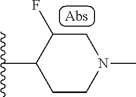 [SMILES: *C1CCN(C)CC1F] | 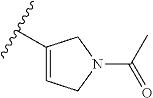 [SMILES: *C1=CCN(C(C)=O)C1] | N-((R)-1-(7-(1-acetyl-2,5- dihydro-1H-pyrrol-3-y1)- 4-aminopyrrolo[2,1- f][1,2,4]triazin-5- yl)piperidin-3-y1)-5- chloro-3-((3-fluoro-1- | 617.2 | E: 1.338 F: 1.053 | (400 MHz, DMSO-d₆) δ ppm 8.74 (br s, 1H), 7.86 (d, J = 1.8 Hz, 1H), 7.39 (s, 1H), 7.26 (br d, J = 5.0 Hz, 1H), 6.80-6.74 (m, 1H), 6.70 (d, J = 13.3 Hz,2H), 4.82-4.54 (m, 3H), 4.51- 4.43 (m, 2H), 4.26 (br d, J = 2.0 Hz, 1H), 4.16- 4.06 (m, 1H), 3.17 (br d, J = 5.3 Hz, 2H), 3.03- |
| | | | methylpiperidin-4- | | | 2.90 (m, 2H), 2.86- 2.73 (m, 2H), 2.60 (dt, J = |
| | | | yl)oxy)thiophene-2- | | | 1.8, 3.4 Hz, 1H), 2.24-2.17 (m, 3H), 2.13-2.05 |
| | | | carboxamide, | | | (m, 2H), 2.02 (d, J = 16.3 Hz, 3H), 1.91 (s, 1H), |
| | | | Diastereomer-2 | | | 1.85-1.77 (m, 2H), 1.71 (br d, J = 3.0 Hz, 1H), |
| | | | | | | 1.52 (br s, 1H); 19F NMR (400 MHz, CDC13) 8 |
| | | | | | | ppm −191.425 |
| 605 | 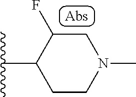 [SMILES: *C1CCN(C)CC1F] | 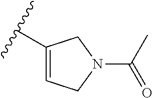 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- yl)piperidin-3-y1)-5- chloro-3-((3-fluoro-1- | 617.2 | E: 1.569 F: 0.985 | (400 MHz, DMSO-d₆) δ ppm 7.87 (d, J = 2.0 Hz, 2H), 7.45 (s, 2H), 6.87-6.70 (m, 3H), 5.08- 4.82 (m, 1H), 4.69 (br s, 2H), 4.48 (br d, J = 6.5 Hz, 2H), 4.27 (br s, 1H), 4.15 (br s, 1H), 3.20- 3.07 (m, 2H), 3.03-2.74 (m, 4H), 2.25-2.15 (m, 4H), 2.06-1.99 (m, 4H), 1.91 (br s, 3H), 1.84 (br |
| | | | methylpiperidin-4- | | | s, 3H) |
| | | | yl)oxy)thiophene-2- | | | |
| | | | carboxamide, | | | |
| | | | Diastereomer-4 | | | |
| 606 | 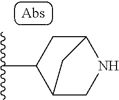 [SMILES: *C1CC2CC1CN2] | 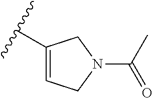 [SMILES: *C1=CCN(C(C)=O)C1] | 3-((2- azabicyclo[2.2.1]heptan- 5-yl)oxy)-N-((R)-1-(7-(1- acetyl-2,5-dihydro-1H- pyrrol-3-y1)-4- aminopyrrolo[2,1- | 597.2 | E: 1.153 F: 0.959 | (400 MHz, DMSO-d₆) δ ppm 8.60 (br s, 1H), 7.86 (s, 1H), 7.32 (s, 2H), 6.80-6.66 (m, 3H), 4.68 (br d, J = 1.0 Hz, 1H), 4.63 (br dd, J = 2.3, 4.8 Hz, 1H), 4.48 (br d, J = 7.0 Hz, 2H), 4.26 (br s, 1H), 4.15 (br s, 1H), 3.08 (br s, 1H), 2.99-2.90 (m, 2H), 2.76 (dt, J = 3.6, 5.1 Hz, 3H), 2.61 (br |
| | | | f][1,2,4]triazin-5~ | | | s, 1H), 2.03 (d, J = 16.5 Hz, 4H), 1.88 (s, 3H), |
| | | | yl)piperidin-3-y1)-5- | | | 1.81 (br s, 3H), 1,65-1,46 (m, 3H), 1.33 (br s, |
| | | | chlorothiophene-2- | | | 1H) |
| | | | carboxamide, | | | |
| | | | Diastereomer-3 | | | |
| 607 | 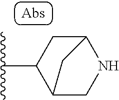 [SMILES: *C1CC2CC1CN2] | 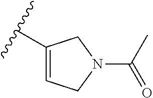 [SMILES: *C1=CCN(C(C)=O)C1] | 3-((2- azabicyclo[2.2.1]heptan- 5-yl)oxy)-N-((R)-1-(7-(1- acetyl-2,5-dihydro-1H- pyrrol-3-y1)-4- aminopyrrolo[2,1- | 597.2 | E: 1.153 F: 0.953 | (400 MHz, DMSO-d₆) δ ppm 8.62 (br s, 1H), 8.32 (br s, 1H), 7.90 (s, 1H), 7.38 (s, 1H), 7.22 (s, 1H), 6.81-6.66 (m, 2H), 4.76-4.67 (m, 2H), 4.53- 4.45 (m, 2H), 4.29-4.24 (m, 1H), 4.18-4.10 (m, 1H), 4.07-4.01 (m, 1H), 3.15-3.04 (m, 3H), 2.97- 2.76 (m, 4H), 2.35-2.31 (m, 2H), 2.03 (d, J = |
| | | | f][1,2,4]triazin-5- | | | 16.0 Hz, 3H), 1.89-1.76 (m, 5H), 1.64-1.51 (m, |
| | | | y1)piperidin-3-y1)-5- | | | 2H) |
| | | | chlorothiophene-2- | | | |
| | | | carboxamide, | | | |
| | | | Diastereomer-4 | | | |
| 608 | 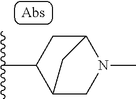 [SMILES: *C1CC2CC1CN2C] | 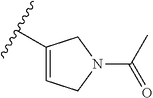 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- y1)piperidin-3-y1)-5- chloro-3-((2-methyl-2- | 611.2 | E: 1.194 F: 0.964 | (400 MHz, DMSO-d₆) δ ppm 9.28 (s, 1H), 7.89 (s, 1H), 7.43-7.33 (m, 1H), 7.24-6.94 (m, 1H), 6.84-6.67 (m, 3H), 4.78-4.65 (m, 2H), 4.53-4.43 (m, 2H), 4.29-3.97 (m, 3H), 3.13-2.71 (m, 9H), 2.16-1.94 (m, 4H), 1.91-1.71 (m, 7H), 1.57 (s, 1H) |
| | | | azabicyclo[2.2.1]heptan- | | | |
| | | | 5-yl)oxy)thiophene-2- | | | |
| | | | carboxamide, | | | |
| | | | Diastereomer-3 | | | |
| 609 | 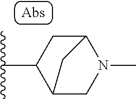 [SMILES: *C1CC2CC1CN2C] | 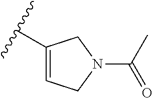 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- yl)piperidin-3-y1)-5- chloro-3-((2-methyl-2- | 611.2 | E: 1.197 F: 0.963 | (400 MHz, DMSO-d₆) δ ppm 10.00-9.30 (m, 1H), 8.23 (s, 1H), 7.91 (s, 1H), 7.44-7.30 (m, 1H), 7.27-6.93 (m, 1H), 6.83-6.66 (m, 2H), 4.79~ 4.61 (m, 2H), 4.55-4.23 (m, 3H), 4.18-4.12 (m, 1H), 4.08-3.97 (m, 1H), 3.35 (br s, 1H), 3.14- 2.72 (m, 8H), 2.43-2.36 (m, 1H), 2.14 -1.97 (m, |
| | | | azabicyclo[2.2.1]heptan- | | | 3H), 1.94-1.70 (m, 7H), 1.58 (br s, 1H) |
| | | | 5-yl)oxy)thiophene-2- | | | |
| | | | carboxamide, | | | |
| | | | Diastereomer-4 | | | |
| 610 | 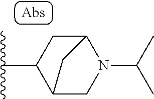 [SMILES: *C1CC2CC1CN2C(C)C] | 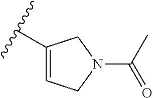 [SMILES: *C1=CCN(C(C)=O)C1] | N-((R)-1-(7-(1-acetyl-2,5- dihydro-1H-pyrrol-3-y1)- 4-aminopyrrolo[2,1- f][1,2,4]triazin-5- y1)piperidin-3-y1)-5- chloro-3-((2-isopropyl-2- | 639.2 | E: 1,288 F: 1.026 | (400 MHz, DMSO-d₆) δ ppm 8.02 (s, 1H), 7.86 (d. J = 2.0 Hz, 1H), 7.35 (d, J = 1.3 Hz, 2H), 6.81-6.51 (m, 3H), 4.68 (br d, J = 2.0 Hz, 1H), 4.58-4.52 (m, 1H), 4.52-4.40 (m, 3H), 4.26 (br d, J = 2.0 Hz, 1H), 4.14 (br s, 1H), 3.10 (br s, 1H), 2.92 (br s, 4H), 2.75 (br s, 1H), 2.42 (br s, 1H), |
| | | | azabicyclo[2.2.1]heptan- | | | 2.03 (d, J = 16.5 Hz, 4H), 1.79 (br s, 5H), 1.51 |
| | | | 5-yl)oxy)thiophene-2- | | | (br s, 3H), 0.95 (br dd, J = 6.4, 11.1 Hz, 6H) |
| | | | carboxamide, | | | |
| | | | Diastereomer-3 | | | |
| 611 | 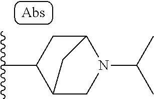 [SMILES: *C1CC2CC1CN2C(C)C] | 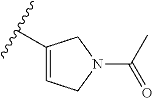 [SMILES: *C1=CCN(C(C)=O)C1] | N-((R)-1-(7-(1-acetyl-2,5- dihydro-1H-pyrrol-3-yl)- 4-aminopyrrolo[2,1- f][1,2,4]triazin-5- yl)piperidin-3-y1)-5- chloro-3-((2-isopropyl-2- | 639.2 | E: 1.290 F: 1.020 | (400 MHz, DMSO-d₆) δ ppm 8.01 (s, 1H), 7.86 (d, J = 2.0 Hz, 1H), 7.35 (d, J = 1.3 Hz, 2H), 6.80-6.65 (m, 3H), 4.71-4.65 (m, 1H), 4.60-4.53 (m, 1H), 4.47 (br d, J = 7.5 Hz, 2H), 4.26 (br d, J = 2.3 Hz, 1H), 4.13 (br s, 1H), 3.11 (br s, 1H), 3.02-2.69 (m, 4H), 2.56 (br s, 1H), 2.39 (br s, |
| | | | azabicyclo[2.2.1]heptan- | | | 2H), 2.03 (d, J = 16.3 Hz, 4H), 1.81 (br s, 4H), |
| | | | 5-yl)oxy)thiophene-2- | | | 1.55 (br s, 4H), 1.03-0.91 (m, 6H) |
| | | | carboxamide, | | | |
| | | | Diastereomer-4 | | | |

### Example 612

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((8-methyl-8-azabicyclo[3.2.1]octan-3-yl)oxy)thiophene-2-carboxamide

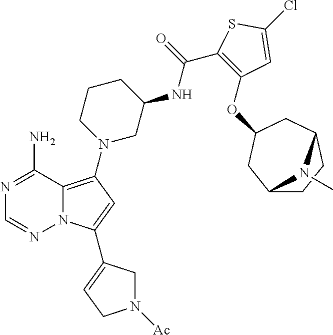 [SMILES: CC(=O)N1CC=C(c2cc(N3CCC[C@@H](NC(=O)c4sc(Cl)cc4O[C@@H]4C[C@H]5CC[C@@H](C4)N5C)C3)c3c(N)ncnn23)C1]

To a solution of Intermediate 32 (61.4 mg, 0.162 mmol) and Intermediate 60 (50 mg. 0.162 mmol) in DMF (2 mL), were added HATU (74.1 mg, 0.195 mmol) and DIEA (0.085 mL, 0.49 mmol). The reaction was stirred at rt for 2 h. The mixture was purified by preparative HPLC to afford Example 612 (16 mg, 15% yield) as a white solid. MS: [M+H]⁺=625.2; HPLC Method I: RT=4.304 min, Method J: RT=4.479 min; ¹H NMR (400 MHz, DMSO-d₆) δ ppm 8.05 (br s, 1H), 7.85 (d, J=2.0 Hz, 1H), 7.41 (d, J=1.5 Hz, 1H), 6.78-6.66 (m, 3H), 4.67 (br s, 2H), 4.47 (br d, J=4.5 Hz, 3H), 4.26 (br s, 3H), 4.18-4.09 (m, 1H), 3.11 (br s, 4H), 2.85 (br s, 3H), 2.13 (s, 3H), 1.99-1.87 (m, 4H), 1.81-1.59 (m, 8H).

### Example 613

### Preparation of methyl 3-(4-amino-5-((R)-3-(5-chloro-3-(((1R,3s,5S)-8-methyl-8-azabicyclo[3.2.1]octan-3-yl)oxy)thiophene-2-carboxamido)piperidin-1-yl)pyrrolo[2,1-f][1,2,4]triazin-7-yl)-2,5-dihydro-1H-pyrrole-1-carboxylate

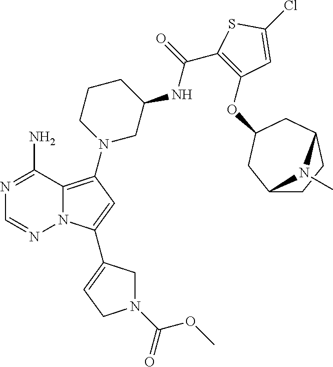 [SMILES: COC(=O)N1CC=C(c2cc(N3CCC[C@@H](NC(=O)c4sc(Cl)cc4O[C@@H]4C[C@H]5CC[C@@H](C4)N5C)C3)c3c(N)ncnn23)C1]

According to the procedure for the preparation of Example 612, coupling Intermediate 31 (64 mg, 0.162 mmol) and Intermediate 60 (50 mg, 0.162 mmol) afforded Example 613 (14 mg, 13% yield) as a white solid. MS: [M+H]⁺=641.2; HPLC Method G: RT=4.221 min, Method J: RT=5.238 min; ¹H NMR (400 MHz, DMSO-d₆) δ ppm 8.31-8.08 (m, 1H), 7.87 (s, 1H), 7.48 (s, 1H), 7.41 (br s, 1H), 6.80-6.69 (m, 3H), 4.74-4.64 (m, 1H), 4.54 (br d, J=11.0 Hz, 2H), 4.29 (br s, 2H), 4.18 (br s, 1H), 3.66 (d, J=4.5 Hz, 3H), 3.29-3.02 (m, 4H), 2.88 (br s, 3H), 2.17 (s, 3H), 1.98 (br d, J=7.0 Hz, 2H), 1.95-1.85 (m, 3H), 1.83-1.76 (m, 2H), 1.70 (br d, J=8.0 Hz, 4H).

### Example 614

### Preparation of N—((R)-1-(4-amino-7-(pyrimidin-5-yl)pyrrolo[2,1-f][1,2,4]triazin-5-yl)piperidin-3-yl)-5-chloro-3-((8-methyl-8-azabicyclo[3.2.1]octan-3-yl)oxy)thiophene-2-carboxamide

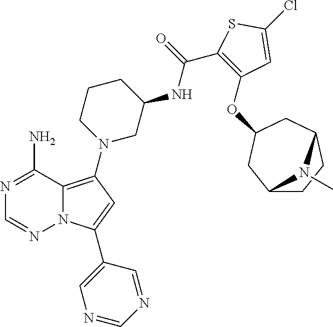 [SMILES: CN1[C@@H]2CC[C@H]1C[C@H](Oc1cc(Cl)sc1C(=O)N[C@@H]1CCCN(c3cc(-c4cncnc4)n4ncnc(N)c34)C1)C2]

According to the procedure for the preparation of Example 612, coupling Intermediate 34A (56.4 mg, 0.162 mmol) and Intermediate 60 (50 mg, 0.162 mmol) afforded Example 614 (26 mg, 25% yield) as a white solid. MS: [M+H]⁺=594.2; HPLC Method G: RT=6.744 min,

Method K: RT=8.330 min, [M+H]⁺=594.2; ¹H NMR (400 MHz, DMSO-d₆) δ ppm 9.47 (s, 2H), 9.09 (s, 1H), 7.93 (s, 1H), 7.48 (s, 1H), 7.33 (s, 1H), 4.74-4.65 (m, 1H), 4.22 (br s, 1H), 3.16-3.09 (m, 2H), 3.01-2.89 (m, 2H), 2.56-2.52 (m, 3H), 2.16 (s, 3H), 2.06-1.95 (m, 2H), 1.95-1.87 (m, 3H), 1.94-1.78 (m, 1H), 1.78-1.55 (m, 6H).

### Example 615

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-cyclopropylpiperidin-4-yl)oxy)thiophene-2-carboxamide

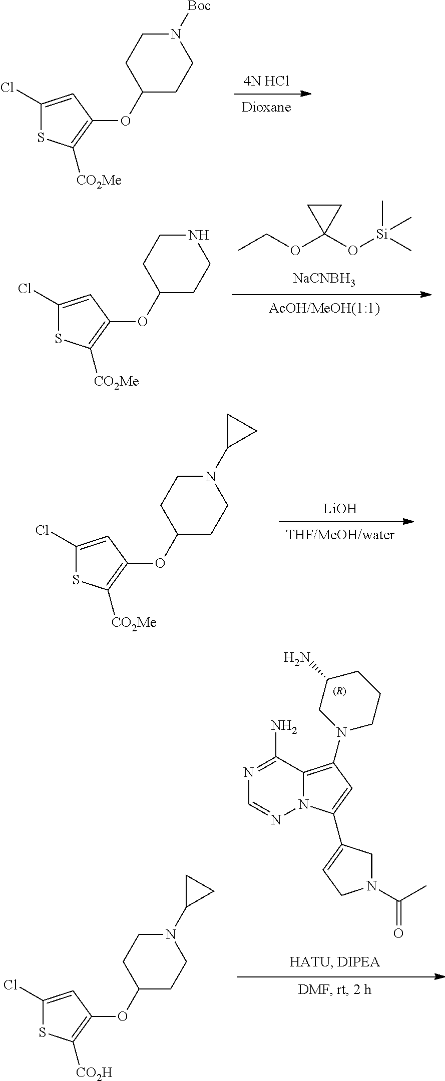

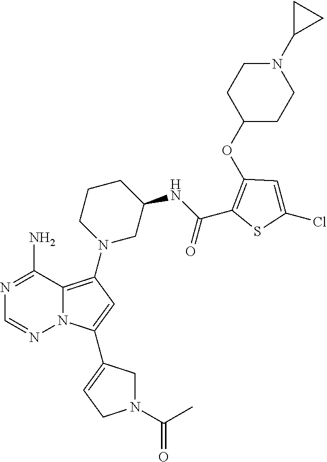 [SMILES: CC(=O)N1CC=C(c2cc(N3CCC[C@@H](NC(=O)c4sc(Cl)cc4OC4CCN(C5CC5)CC4)C3)c3c(N)ncnn23)C1]

### Example 615A. Methyl 5-chloro-3-(piperidin-4-yloxy)thiophene-2-carboxylate

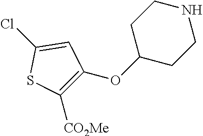 [SMILES: COC(=O)c1sc(Cl)cc1OC1CCNCC1]

To a solution of Example 561A (1.0 g, 2.66 mmol) in dioxane (5 mL), was added 4 N HCl in dioxane (6.65 mL, 26.6 mmol). The mixture was stirred at rt for 4 h and concentrated. The residue was washed with diethyl ether (2×20 mL) and dried under vacuum to afford Example 615A (0.7 g, 84% yield) as a white solid. MS: [M+H]⁺=276.0; ¹H NMR (400 MHz, DMSO-d₆) δ ppm 7.44 (s, 1H), 4.85-4.71 (m, 1H), 3.76 (s, 3H), 3.23-2.99 (m, 4H), 2.13-1.84 (m, 4H).

### Example 615B. Methyl 5-chloro-3-((1-cyclopropylpiperidin-4-yl)oxy)thiophene-2-carboxylate

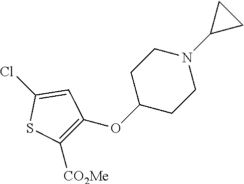 [SMILES: COC(=O)c1sc(Cl)cc1OC1CCN(C2CC2)CC1]

To a solution of Example 615A (250 mg, 0.801 mmol) and (1-ethoxycyclopropoxy)-trimethylsilane, (698 mg, 4.00 mmol) in methanol (2 mL) and acetic acid (2 mL), was added sodium cyanoborohydride (101 mg, 1.60 mmol). It was heated at 70° C. for 14 h and concentrated. The residue was diluted with DCM and washed with brine. The organic layer was dried over Na₂SO₄, filtered and concentrated to afford Example 615B (200 mg, 79% yield) as a yellow thick liquid. MS: [M+H]⁺=316.1; ¹H NMR (400 MHz, CDCl₃) δ ppm 6.77-6.71 (m, 1H), 4.41-4.29 (m, 1H), 3.92 (s, 3H), 3.04-2.84 (m, 2H), 2.71-2.49 (m, 3H), 2.03-1.82 (m, 4H), 0.53-0.37 (m, 4H).

### Example 615C. 5-chloro-3-((1-cyclopropylpiperidin-4-yl)oxy)thiophene-2-carboxylate, lithium salt

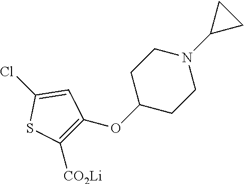

Analogous to the procedure for the preparation of Example 346B, reaction of Example 615B (200 mg, 0.633 mmol) and lithium hydroxide monohydrate (53.1 mg, 1.27 mmol) afforded Example 615C (160 mg, 82% yield) as a white solid. MS: [M+H]⁺=302.2; ¹H NMR (400 MHz, DMSO-d₆) δ ppm 6.77 (s, 1H), 4.64-4.43 (m, 1H), 2.83 (br dd, J=5.0, 10.5 Hz, 2H), 2.46-2.21 (m, 2H), 1.89-1.72 (m, 2H), 1.64-1.46 (m, 3H), 0.44-0.21 (m, 4H).

### Example 615

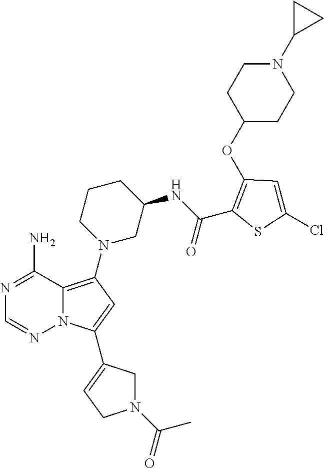 [SMILES: CC(=O)N1CC=C(c2cc(N3CCC[C@@H](NC(=O)c4sc(Cl)cc4OC4CCN(C5CC5)CC4)C3)c3c(N)ncnn23)C1]

To a solution of Intermediate 32 (61.4 mg, 0.162 mmol) and Example 615C (50 mg, 0.162 mmol) in DMF (2 mL), were added HATU (74.1 mg, 0.195 mmol) and DIEA (0.085 mL, 0.49 mmol). The reaction was stirred at rt for 2 h and water was added. The precipitate was collected and purified by preparative HPLC to afford Example 615 (25 mg, 25% yield) as a pale yellow solid. LC-MS Method E: RT=1.845 min, [M+H]⁺=625.2; LC-MS Method F: RT=1.044 min, [M+H]⁺=625.3; ¹H NMR (400 MHz, DMSO-d₆) δ ppm 8.16-8.05 (m, 1H), 7.89 (s, 1H), 7.50-7.42 (m, 1H), 7.37-7.27 (m, 1H), 6.84-6.64 (m, 3H), 4.78-4.61 (m, 2H), 4.53-4.39 (m, 3H), 4.32-4.10 (m, 2H), 3.69-3.56 (m, 2H), 3.21-2.93 (m, 6H), 2.89-2.72 (m, 3H), 2.42-2.37 (m, 1H), 2.24-2.13 (m, 1H), 2.06-2.00 (m, 3H), 1.93-1.71 (m, 5H), 1.58-1.28 (m, 2H).

The following examples in Table 45 were prepared using the same procedure as shown for Example 615.

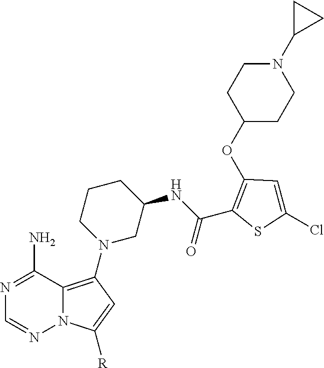

TABLE 45

| | | | | HPLC | |
|---|---|---|---|---|---|
| | | | LCMS | Method, | |
| Example | R | Name | (M + H)⁺ | RT (min.) | ¹H NMR |
| 616 | 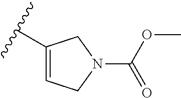 [SMILES: *C1=CCN(C(=O)OC)C1] | methyl (R)-3-(4-amino-5- (3-(5-chloro-3-((1- cyclopropylpiperidin-4- yl)oxy)thiophene-2- carboxamido)piperidin-1- yl)pyrrolo[2,1- | 641.2 | E: 2.136 F: 1.231 | (400 MHz, DMSO-d₆) δ ppm 7.85 (s, 2H), 7.39 (s, 2H), 6.80-6.56 (m, 3H), 4.66-4.41 (m, 3H), 4.37-4.22 (m, 2H), 4.22-4.03 (m, 1H), 3.65 (d, J = 4.3 Hz, 3H), 3.19-3.08 (m, 2H), 2.97-2.61 (m, 5H), 1.99-1.74 (m, 5H), 1.71-1.33 (m, 4H), 0.45-0.10 (m, 5H) |
| | | f][1,2,4]triazin-7-yl)-2,5- | | | |
| | | dihydro-1H-pyrrole-1- | | | |
| | | carboxylate | | | |
| 617 | 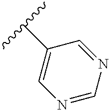 [SMILES: *c1cncnc1] | (R)-N-(1-(4-amino-7- (pyrimidin-5- y1)pyrrolo[2,1- f][1,2,4]triazin-5- yl)piperidin-3-y1)-5- chloro-3-((1- cyclopropylpiperidin-4- | 594.2 | E: 1.916 F: 1.028 | (400 MHz, DMSO-dc) 8 ppm 9.47 (s, 2H), 9.18-9.03 (m, 1H), 7.99-7.87 (m, 1H), 7.52- 7.26 (m, 3H), 4.90-4.60 (m, 1H), 4.33-4.08 (m, 1H), 3.71-3.57 (m, 2H), 3.27-3.11 (m, 1H), 3.12-2.78 (m, 6H), 2.26-2.13 (m, 1H), 2.09-1.70 (m, 6H), 1.66-1.45 (m, 1H), 1.01-0.72 (m, 5H) |
| | | yl)oxy)thiophene-2- | | | |
| | | carboxamide | | | |

### Example 618

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1-(2-hydroxy-2-methylpropyl)piperidin-4-yl)oxy)thiophene-2-carboxamide

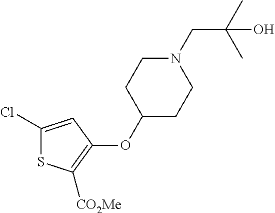 [SMILES: COC(=O)c1sc(Cl)cc1OC1CCN(CC(C)(C)O)CC1]

### Example 618A. Methyl 5-chloro-3-((1-(2-hydroxy-2-methylpropyl)piperidin-4-yl)oxy)thiophene-2-carboxylate

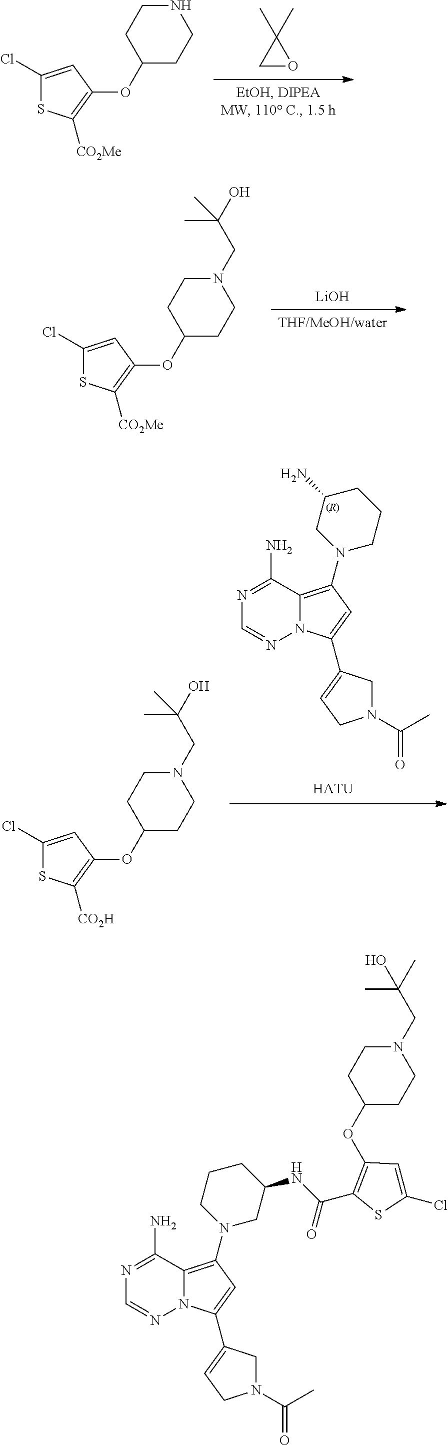

### Example 618B. 5-chloro-3-((1-(2-hydroxy-2-methylpropyl)piperidin-4-yl)oxy)thiophene-2-carboxylic acid

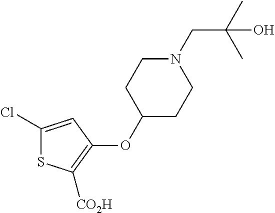 [SMILES: CC(C)(O)CN1CCC(Oc2cc(Cl)sc2C(=O)O)CC1]

Analogous to the procedure for the preparation of Example 346B, reaction of Example 618A and 2 equiv. LiOH afforded Example 618B.

### Example 618

According to the procedure for the preparation of Example 616, coupling of Example 618B with Intermediate 32 afforded Example 619. LC-MS Method E: RT=1.546 min, [M+H]⁺=657.3; LC-MS Method F: RT=0.992 min, [M+H]⁺=657.3; (400 MHz, DMSO-d₆) δ ppm 7.86 (d, J=2.3 Hz, 2H), 7.38 (d, J=1.0 Hz, 2H), 6.82-6.54 (m, 3H), 4.68 (br d, J=1.5 Hz, 1H), 4.63-4.54 (m, 1H), 4.47 (br d, J=10.3 Hz, 2H), 4.26 (br s, 1H), 4.19-3.95 (m, 2H), 2.99-2.71 (m, 3H), 2.68-2.61 (m, 2H), 2.46-2.34 (m, 2H), 2.17-2.06 (m, 2H), 2.02 (d, J=15.8 Hz, 3H), 1.95-1.77 (m, 6H), 1.75-1.45 (m, 3H), 1.01 (br s, 6H).

### Example 619

### Methyl (R)-3-(4-amino-5-(3-(5-chloro-3-((1-(2-hydroxy-2-methylpropyl)piperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

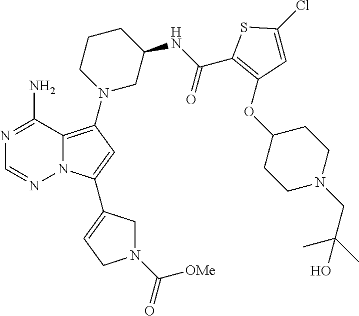 [SMILES: COC(=O)N1CC=C(c2cc(N3CCC[C@@H](NC(=O)c4sc(Cl)cc4OC4CCN(CC(C)(C)O)CC4)C3)c3c(N)ncnn23)C1]

According to the procedure for the preparation of Example 616, coupling of Example 618B with Intermediate 31 afforded Example 619. LC-MS Method E: RT=1.813 min, [M+H]⁺=672.2; LC-MS Method F: RT=1.135 min, [M+H]⁺=672.2; (400 MHz, DMSO-d₆) δ ppm 7.86 (d, J=2.3 Hz, 2H), 7.38 (d, J=1.0 Hz, 2H), 6.82-6.54 (m, 3H), 4.68 (br d, J=1.5 Hz, 1H), 4.63-4.54 (m, 1H), 4.47 (br d, J=10.3 Hz, 2H), 4.26 (br s, 1H), 4.19-3.95 (m, 2H), 2.99-2.71 (m, 3H), 2.68-2.61 (m, 2H), 2.46-2.34 (m, 2H), 2.17-2.06 (m, 2H), 2.02 (d, J=15.8 Hz, 3H), 1.95-1.77 (m, 6H), 1.75-1.45 (m, 3H), 1.01 (br s, 6H).

### Example 621

### Preparation of methyl 3-(4-amino-5-((3R)-3-(5-chloro-3-((2-methylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate

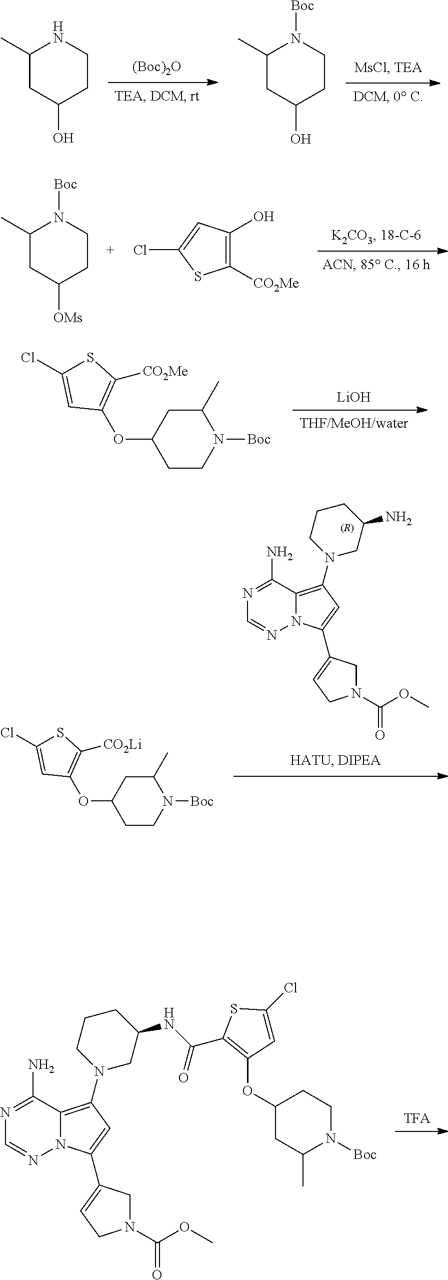

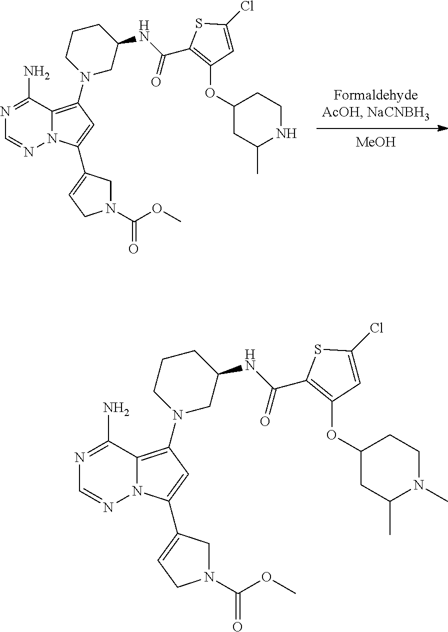 [SMILES: "MeOH" ; COC(=O)N1CC=C(c2cc(N3CCC[C@@H](NC(=O)c4sc(Cl)cc4OC4CCNC(C)C4)C3)c3c(N)ncnn23)C1 ; "FormaldehydeAcOH, NaCNBH3" ; COC(=O)N1CC=C(c2cc(N3CCC[C@@H](NC(=O)c4sc(Cl)cc4OC4CCN(C)C(C)C4)C3)c3c(N)ncnn23)C1]

### Example 621A. tert-butyl 4-hydroxy-2-methylpiperidine-1-carboxylate

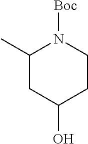

To a solution of 2-methylpiperidin-4-ol (1.0 g, 8.68 mmol) in DCM (10 mL) at 0° C., were added TEA (2.42 mL, 17.4 mmol) and Boc-anhydride (2.02 mL, 8.68 mmol) dropwise and the mixture was stirred at it for 3 b. The reaction was quenched with sat. NH₄Cl solution and extracted in DCM. DCM layer was washed with brine solution, dried over Na₂SO₄, filtered and concentrated to afford Example 621A (1.8 g, 96% yield) as a yellow liquid; ¹H NMR (400 MHz, CDCl₃) δ ppm 4.54-4.44 (m, 1H), 4.08-3.90 (m, 2H), 2.92-2.82 (m, 1H), 2.67-2.58 (m, 2H), 1.97-1.77 (m, 2H), 1.71-1.62 (m, 1H), 1.46 (s, 9H), 1.14 (d, J=7.3 Hz, 3H).

### Example 621B. tert-butyl 2-methyl-4-((methylsulfonyl)oxy)piperidine-1-carboxylate

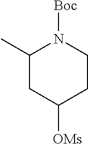 [SMILES: CC1CC(OS(C)(=O)=O)CCN1C(=O)OC(C)(C)C]

To a solution of Example 621A (1.8 g 8.36 mmol) in DCM (20 mL), was added TEA (2.33 mL, 16.7 mmol) and Ms-Cl (0.782 mL, 10.0 mmol) at 0° C. The mixture was stirred at rt for 3 h, diluted with DCM (20 mL) and washed with sat. NaHCO₃ solution (50 mL). DCM layer was separated, dried over Na₂SO₄, filtered and concentrated to afford Example 621B (2.3 g, 94% yield) as a yellow thick liquid; ¹H NMR (300 MHz, CDCl₃) δ ppm 5.02-4.86 (m, 1H), 4.62-4.47 (m, 1H), 4.17-4.02 (m, 1H), 3.03 (s, 3H), 2.99-2.85 (m, 1H), 2.19-1.93 (m, 2H), 1.88-1.58 (m, 2H), 1.46 (s, 9H), 1.18 (d, J=7.3 Hz, 3H).

### Example 621C. tert-butyl 4-((5-chloro-2-(methoxycarbonyl)thiophen-3-yl)oxy)-2-methylpiperidine-1-carboxylate

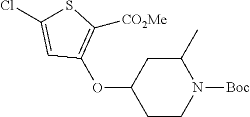 [SMILES: COC(=O)c1sc(Cl)cc1OC1CCN(C(=O)OC(C)(C)C)C(C)C1]

Analogous to the procedure for the preparation of Example 347A, reaction of methyl 5-chloro-3-hydroxythiophene-2-carboxylate (500 mg, 2.60 mmol) and Example 621B (190 mg, 6.49 mmol) afforded Example 621C (550 mg, 54% yield) as a yellow liquid. MS: [M+H-t-Bu]⁺=334.2; ¹H NMR (400 MHz, CDCl₃) δ ppm 6.73 (s, 1H), 4.68-4.60 (m, 1H), 4.44-4.30 (m, 1H), 3.96-3.88 (m, 1H), 3.83 (s, 3H), 3.42-3.29 (m, 1H), 2.02-1.91 (m, 3H), 1.81-1.64 (m, 1H), 1.49 (s, 9H), 1.44-1.36 (m, 3H).

### Example 621D. 3-((1-(tert-butoxycarbonyl)-2-methylpiperidin-4-yl)oxy)-5-chlorothiophene-2-carboxylic acid, lithium salt

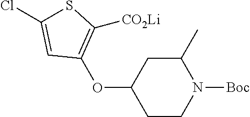

To a solution of Example 621C (550 mg, 1.411 mmol) in THF (1 mL) and methanol (1 mL), were added lithium hydroxide (118 mg, 2.82 mmol) and water (1 mL). The mixture was stirred at rt for 14 h and concentrated to afford Example 621D (950 mg, 97% yield) as a yellow solid. MS: [M+H]⁺=376.0; ¹H NMR (400 MHz, DMSO-d₆) δ ppm 6.84 (s, 1H), 4.84-4.77 (m, 1H), 4.22-4.11 (m, 1H), 3.69-3.57 (m, 1H), 1.91-1.69 (m, 3H), 1.56-1.43 (m, 1H), 1.40 (s, 9H), 1.38-1.38 (m, 1H), 1.32 (d, J=7.0 Hz, 3H).

### Example 621E, tert-butyl 4-((2-(((R)-1-(4-amino-7-(1-(methoxycarbonyl)-2,5-dihydro-1H-pyrrol-3-yl)pyrrolo[2,1-f][1,2,4]triazin-5-yl)piperidin-3-yl)carbamoyl)-5-chlorothiophen-3-yl)oxy)-2-methylpiperidine-1-carboxylate (Diastereomeric mixture)

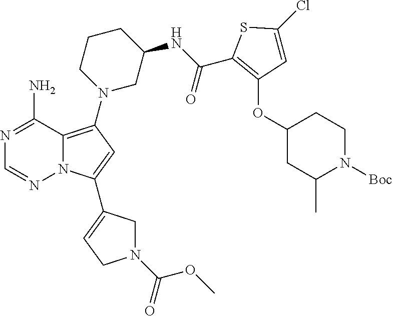 [SMILES: COC(=O)N1CC=C(c2cc(N3CCC[C@@H](NC(=O)c4sc(Cl)cc4OC4CCN(C(=O)OC(C)(C)C)C(C)C4)C3)c3c(N)ncnn23)C1]

To a solution of Intermediate 31 (155 mg, 0.393 mmol) and Example 621D (150 mg, 0.393 mmol) in DMF (5 mL), were added HATU (179 mg, 0.471 mmol) and DIEA (0.206 mL, 1.179 mmol). The mixture was stirred at it for 2 h followed by the addition of cold water. Precipitated solid was filtered, washed with water, dried by vacuum to afford Example 621E (Diastereomeric mixture) as a yellow solid (200 mg). It was purified by preparative HPLC to afford two diastereomers.

Diastereomer-1: Example 621F (30 mg, 11% yield) as a white solid, MS: [M+H-t-Bu]⁺=715.3; ¹H NMR (400 MHz, DMSO-d₆) δ ppm 7.99 (br s, 1H), 7.86 (s, 1H), 7.34 (s, 1H), 6.75-6.68 (m, 3H), 4.79 (br s, 1H), 4.55 (br s, 2H), 4.29 (br s, 2H), 4.13 (br s, 2H), 3.70-3.63 (m, 3H), 3.45-3.34 (m, 4H), 3.22-2.86 (m, 2H), 2.33-2.21 (m, 1H), 1.93 (br s, 1H), 1.85 (br s, 4H), 1.77 (br s, 2H), 1.33 (s, 9H), 1.10 (br d, J=6.5 Hz, 3H).

Diastereomer-2: Example 621G (30 mg, 11% yield) as an off-white solid, MS: [M+H-t-Bu]⁺=715.3; ¹H NMR (400 MHz, DMSO-d₆) δ ppm 8.01 (br s, 1H), 7.86 (s, 1H), 7.35 (s, 1H), 6.78-6.66 (m, 3H), 4.91-4.74 (m, 1H), 4.60-4.48 (m, 2H), 4.34-4.10 (m, 4H), 3.86-3.75 (m, 3H), 3.70-3.61 (m, 3H), 3.44-3.34 (m, 1H), 3.22-2.91 (m, 3H), 2.02-1.74 (m, 7H), 1.40 (s, 9H), 1.10 (br d, J=6.5 Hz, 3H).

### Example 621H. Methyl 3-(4-amino-5-((3R)-3-(5-chloro-3-((2-methylpiperidin-4-yl)oxy)thiophene-2-carboxamido)piperidin-1-yl)pyrrolo[2,1-f][1,2,4]triazin-7-yl)-2,5-dihydro-1H-pyrrole-1-carboxylate (Diastereomeric mixture)

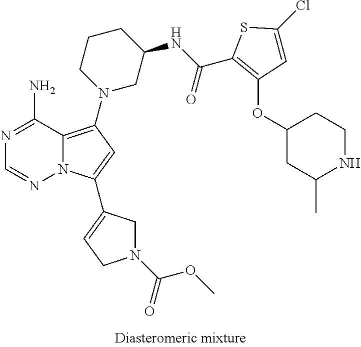 [SMILES: COC(=O)N1CC=C(c2cc(N3CCC[C@@H](NC(=O)c4sc(Cl)cc4OC4CCNC(C)C4)C3)c3c(N)ncnn23)C1]

Reaction of Example 621E (Diastereomeric mixture) (100 mg, 0.140 mmol) and TFA (0.215 mL, 2.80 mmol) afforded Example 621H (diastereomeric mixture) as a yellow solid. MS: [M+H]⁺=615.2; ¹H NMR (400 MHz, DMSO-d₆) δ ppm 8.42 (br s, 2H), 7.43 (s, 1H), 7.28 (s, 1H), 6.85-6.75 (m, 1H), 6.86-6.72 (m, 1H), 4.66 (br s, 3H), 4.30 (br s, 2H), 4.16 (br s, 3H), 3.46-3.31 (m, 2H), 3.25 (br s, 1H), 3.21-3.05 (m, 2H), 2.94 (br d, J=11.0 Hz, 4H), 2.83-2.71 (m, 1H), 2.42-2.17 (m, 2H), 1.81 (br s, 3H), 1.67 (br s, 3H), 1.28-1.22 (m, 3H).

### Example 621

Analogous to the procedure for the preparation of Example 376, reaction of Example 621H (diastereomeric mixture) (100 mg, 0.137 mmol) and formaldehyde solution (0.054 mL, 0.686 mmol) afforded Example 621 (diastereomeric mixture) as a white solid. LCMS Method G: RT=4.605 min; Method H: RT=5.644 min; LC-MS [M+H]⁺=629.2; ¹H NMR (400 MHz, DMSO-d₆) δ ppm 8.03 (br s, 1H), 7.86 (d, J=1.5 Hz, 1H), 7.43 (d, J=2.8 Hz, 2H), 6.82-6.64 (m, 3H), 4.58-4.40 (m, 3H), 4.29 (br s, 2H), 4.15 (br s, 1H), 3.66 (d, J=4.3 Hz, 3H), 3.10 (br d, J=10.0 Hz, 2H), 2.90 (br s, 2H), 2.79 (br d, J=6.0 Hz, 2H), 2.13 (s, 4H), 1.80 (s, 2H), 1.57 (br d, J=10.8 Hz, 3H), 1.44-1.16 (m, 2H), 1.06-0.84 (m, 4H).

The following examples in Table 47 were prepared using the same procedure as shown in Example 621.

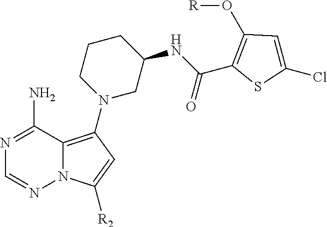

TABLE 47

| | | | | | | HPLC | |
|---|---|---|---|---|---|---|---|
| | | | | | LCMS | Method, | |
| Example | Stercochemistry | R | R₂ | Name | (M + H)⁺ | RT (min.) | ¹H NMR |
| 622 | Homochiral (Diastereomer-1) | 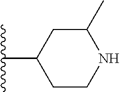 [SMILES: *C1CCNC(C)C1] | 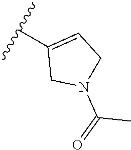 [SMILES: *C1=CCN(C(C)=O)C1] | N-((R)-1-(7-(1-acetyl- 2,5-dihydro-1H-pyrrol-3- y1)-4-aminopyrrolo[2,1- f\|[1,2,4]triazin-5- y1)piperidin-3-y1)-5- chloro-3-((2- methylpiperidin-4- yl)oxy)thiophone-2- carboxamide | 599.2 | E: 1.288 F: 1.013 | (400 MHz, DMSO-d₆) δ ppm 8.03 (br s, 1H), 7.87 (s, 1H), 7.41-7.36 (m, 2H), 6.74 (d, J = 13.3 Hz, 3H), 4.69 (br d, J = 3.0 Hz, 1H), 4.54- 4.42 (m, 3H), 4.27 (br s, 1H), 4.16 (br s, 1H), 3.16-3.11 (m, 1H), 3.01- 2.88 (m, 3H), 2.57 (br d, J = 1.5 Hz, 3H), 2.03 (d, J = 16.0 Hz, 6H), 1.91 (s, 1H), 1.82 (br s, 1H), 1.86- |
| | | | | | | | 1.76 (m, 1H), 1.38 (s, 1H), 1.12- |
| | | | | | | | 1.01 (m, 1H), 0.95 (br d, J = 5.3 |
| | | | | | | | Hz, 3H) |
| 623 | Homochiral (Diastereomer-2) | 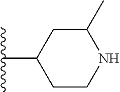 [SMILES: *C1CCNC(C)C1] | 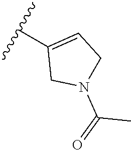 [SMILES: *C1=CCN(C(C)=O)C1] | N-((R)-1-(7-(1-acetyl- 2,5-dihydro-1H-pyrrol-3- yl)-4-aminopyrrolo[2,1- f][1,2,4]triazin-5- yl)piperidin-3-y1)-5- chloro-3-((2- methylpiperidin-4- yl)oxy)thiophene-2- carboxamide | 599.2 | E: 1.274 F: 1.015 | (400 MHz, DMSO-d₆) δ ppm 8.03 (br s, 1H), 7.87 (d, J = 2.3 Hz, 1H), 7.42-7.30 (m, 2H), 6.80-6.67 (m, 3H), 4.69 (br s, 1H), 4.54-4.42 (m, 3H), 4.27 (br d, J = 1.5 Hz, 1H), 4.15 (br s, 1H), 3.15-3.09 (m, 1H), 2.97-2.77 (m, 4H), 2.64-2.55 (m, 2H), 2.03 (d, J = 16.5 Hz, 6H), 1.91 (s, 1H), 1.82 (br dd, J = 1.8, 3.5 Hz, |
| | | | | | | | 3H), 1.60 (s, 1H), 1.35 (br d, J = |
| | | | | | | | 1.8 Hz, 1H), 0,97-0.88 (m, 3H) |
| 624 | Homochiral (Diastercomer-1) | 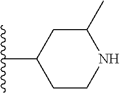 [SMILES: *C1CCNC(C)C1] | 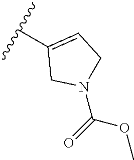 [SMILES: *C1=CCN(C(=O)OC)C1] | Methyl 3-(4-amino-5- ((3R)-3-(5-chloro-3-((2- methylpiperidin-4- yl)oxy)thiophene-2- carboxamido)piperidin- 1-yl)pyrrolo[2,1- f][1,2,4]triazin-7-y1)-2,5- dihydro-1H-pyrrole-1- carboxylate | 615.3 | E: 1.474 F: 1.161 | (400 MHz, DMSO-d₆) δ ppm 8.59 (br s, 1H), 8.34-8.22 (m, 1H), 6.79- 6.68 (m, 3H), 4.64 (s, 1H), 4.57- 4.48 (m, 2H), 4.35-4.25 (m, 2H), 4.16 (br s, 1H), 4.20-4.09 (m, 1H), 3.65 (d, J = 4.5 Hz, 3H), 3.28-3.22 (m, 2H), 3.13 (br s, 1H), 3.03-2.92 (m, 2H), 2.82 (br s, 2H), 2.32-2.25 (m, 2H), 1.93-1.75 (m, 3H), 1.66 (br dd, J = 2.6, 11.9 Hz, 1H), 1.57- |
| | | | | | | | 1.44 (m, 3H), 1.25-1.18 (m, 4H) |
| 625 | Homochiral (Diastereomer-1) | 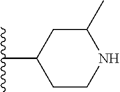 [SMILES: *C1CCNC(C)C1] | 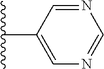 [SMILES: *c1cncnc1] | N-((R)-1-(4-amino-7- (pyrimidin-5- y1)pyrrolo[2,1- f][1,2,4]triazin-5- y1)piperidin-3-y1)-5- chloro-3-((2- | 568.2 | E: 1.237 F: 0.999 | (400 MHz, DMSO-d₆) δ ppm 9.46 (s, 2H), 9.08 (s, 1H), 7.91 (s, 1H), 7.41-7.36 (m, 2H), 7.31 (s, 1H), 4.50-4.46 (m, 1H), 4.23-4.16 (m, 1H), 3.01-2.82 (m, 5H), 2.64-2.55 (m, 2H), 2.07 (s, 2H), 1.93-1.77 (m, |
| | | | | methylpiperidin-4- | | | 4H), 1.64-1.48 (m, 1H), 1.38 (br d, |
| | | | | yl)oxy)thiophene-2- | | | J = 11.0 Hz, 1H), 1.07 (br d, J = 12.0 |
| | | | | carboxamide | | | Hz, 1H). 0.94 (br d, J = 6.3 Hz, 3H) |
| 626 | Homochiral (Diastereomer-2) | 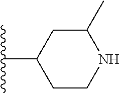 [SMILES: *C1CCNC(C)C1] | 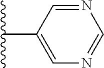 [SMILES: *c1cncnc1] | N-((R)-1-(4-amino-7- (pyrimidin-5- yl)pyrrolo[2,1- f][1,2,4]triazin-5- y1)piperidin-3-y1)-5- chloro-3-((2- | 568.2 | E: 1.225 F: 1.002 | (400 MHz, DMSO-d₆) δ ppm 9.46 (s, 2H), 9.08 (s, 1H), 7.91 (s, 1H), 7.41-7.38 (m, 2H), 7.31 (s, 1H), 4.53-4.44 (m, 1H), 4.22-4.10 (m, 1H), 3.17 (br s, 1H), 3.00-2.80 (m, 5H), 2.63-2.56 (m, 2H), 2.07-2.00 |
| | | | | methylpiperidin-4- | | | (m, 2H), 1.89-1.73 (m, 3H), 1.69- |
| | | | | yl)oxy)thiophene-2- | | | 1.46 (m, 1H), 1.41-1.27 (m, 1H), |
| | | | | carboxamide | | | 1.07 (br d, J = 12.0 Hz, 1H), 0.94 |
| | | | | | | | (br d, J = 6.0 Hz, 3H) |
| 627 | Diastereomer mixture | 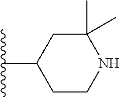 [SMILES: *C1CCNC(C)(C)C1] | 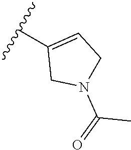 [SMILES: *C1=CCN(C(C)=O)C1] | N-((R)-1-(7-(1-acetyl- 2,5-dihydro-1H-pyrrol-3- y1)-4-aminopyrrolo[2,1- f][1,2,4]triazin-5- yl)piperidin-3-y1)-5- chloro-3-((2,2- dimethylpiperidin-4- yl)oxy)thiophene-2- carboxamide | 613.2 | I: 6.646 J: 5.796 | (400 MHz, DMSO-d₆) δ ppm 8.03 (br s, 1H), 7.87 (s, 1H), 7.40 (s, 2H), 6.86-6.65 (m, 3H), 4.73-4.59 (m, 2H), 4.48 (br d, J = 8.3 Hz, 2H), 4.32-4.06 (m, 2H), 3.18-3.10 (m, 1H), 2.96-2.66 (m, 6H), 2.03 (d, J = 16.1 Hz, 4H), 1.93-1.74 (m, 4H), 1.66-1.41 (m, 1H), 1.34-1.13 (m, 2H), 1.09-0.84 (m, 6H) |
| 628 | diastereomer mixture | 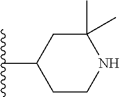 [SMILES: *C1CCNC(C)(C)C1] | 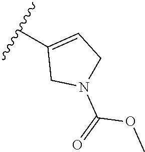 [SMILES: *C1=CCN(C(=O)OC)C1] | methyl 3-(4-amino-5- ((3R)-3-(5-chloro-3- ((2,2-dimethylpiperidin- 4-yl)oxy)thiophene-2- carboxamido)piperidin- 1-yl)pyrrolo[2,1- f][1,2,4]triazin-7-y1)-2,5- dihydro-1H-pyrrole-1- carboxylate | 629.2 | G: 5.123 K: 5.585 | (400 MHz, DMSO-d₆) δ ppm 8.08 (br s, 1H),7.90-7.82 (m, 1H), 7.45 (br s, 2H), 6.81-6.69 (m, 3H), 4.72- 4.49 (m, 3H), 4.36-4.09 (m, 3H), 3.70-3.63 (m, 3H), 3.18-3.09 (m, 1H), 2.97-2.70 (m, 5H), 2.07-1.76 (m, 5H), 1.67-1,45 (m, 2H), 1.36- 1.11 (m, 2H), 1.07-0.90 (m, 6H) |
| 629 | Homochiral (Diastereomer-1) | 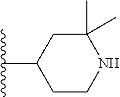 [SMILES: *C1CCNC(C)(C)C1] | 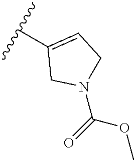 [SMILES: *C1=CCN(C(=O)OC)C1] | methyl 3-(4-amino-5- ((3R)-3-(5-chloro-3- ((2,2-dimethylpiperidin- 4-yl)oxy)thiophene-2- carboxamido)piperidin- 1-y1)pyrrolo[2,1- f][1,2,4]triazin-7-yl)-2,5- dihydro-1H-pyrrole-1- carboxylate | 629.2 | G: 4.689 K: 5.733 | (400 MHz, DMSO-d₆) δ ppm 8.05 (br s, 1H), 7.91-7.82 (m, 1H), 7.55- 7.34 (m, 2H), 6.81-6.68 (m, 3H), 4.66 (s, 3H), 4.37-4.09 (m, 3H), 3.72-3.62 (m, 3H), 2.76 (br s, 4H), 1.95-1.82 (m, 8H), 1.30-1.15 (m, 3H), 1.05 (s, 6H) |
| 630 | Homochiral (Diastereomer-2) | 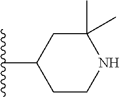 [SMILES: *C1CCNC(C)(C)C1] | 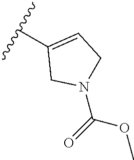 [SMILES: *C1=CCN(C(=O)OC)C1] | methyl 3-(4-amino-5- ((3R)-3-(5-chloro-3- ((2,2-dimethylpiperidin- 4-yl)oxy)thiophene-2- carboxamido)piperidin- 1-yl)pyrrolo[2,1- f][1,2,4]triazin-7-y1)-2,5- dihydro-1H-pyrrole-1- carboxylate | 629.2 | G: 4.640 K: 5.711 | (400 MHz, DMSO-d₆) δ ppm 8.06 (br s, 1H), 7.86 (s, 1H), 7.41 (s, 2H), 6.83-6.68 (m, 3H), 4.72-4.48 (m, 3H), 4.37-4.08 (m, 3H), 3.66 (d, J = 3.9 Hz, 3H), 2.94-2.70 (m, 4H), 1.95-183 (m, 8H), 1.24-118 (s, 3H), 1.04 (s, 6H) |

### Example 631

### Preparation of (R)—N-(1-(4-amino-7-(4-cyano-3-fluorophenyl)pyrrolo[2,1-f][1,2,4]triazin-5-yl)piperidin-3-yl)-5-chloro-3-((2-isopropyl-2-azaspiro[3.3]heptan-6-yl)oxy)thiophene-2-carboxamide

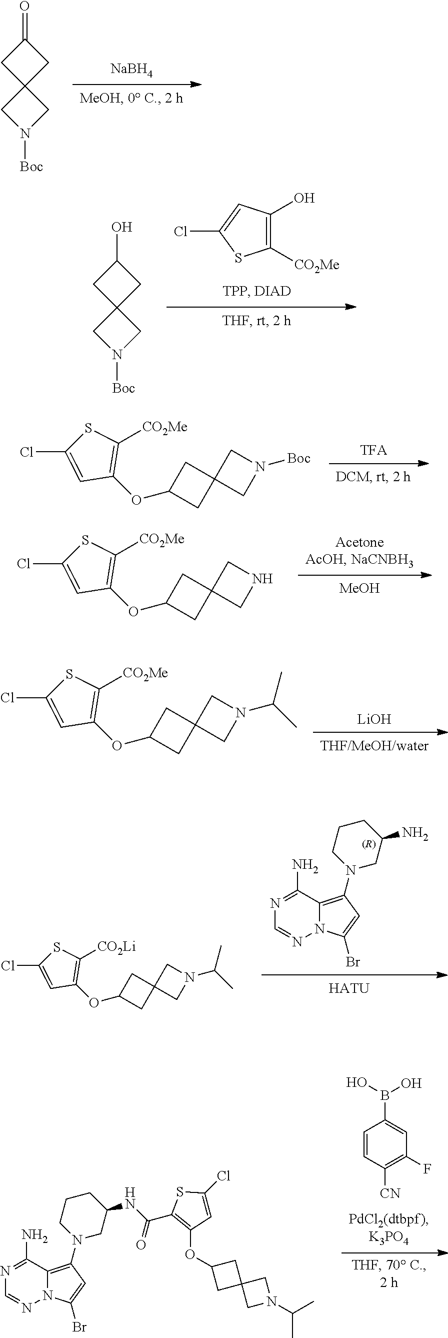

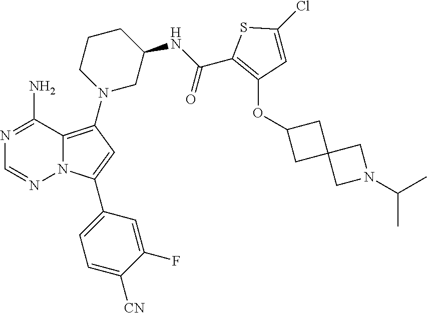 [SMILES: CC(C)N1CC2(CC(Oc3cc(Cl)sc3C(=O)N[C@@H]3CCCN(c4cc(-c5ccc(C#N)c(F)c5)n5ncnc(N)c45)C3)C2)C1]

### Example 631A. tert-butyl 6-hydroxy-2-azaspiro[3.3]heptane-2-carboxylate

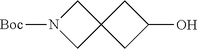 [SMILES: CC(C)(C)OC(=O)N1CC2(CC(O)C2)C1]

To a solution of tert-butyl 6-oxo-2-azaspiro[3.3]heptane-2-carboxylate (5 g, 23.67 mmol) in methanol (50 ml), was added NaBH₄ (1.791 g, 47.3 mmol) at 0° C. The mixture was stirred at rt for 2 h and concentrated. The residue was suspended in DCM (120 mL). followed by the addition of aq. sat. NaHCO₃ (100 mL). DCM layer was separated, dried over Na₂SO₄, filtered and concentrated to afford Example 631A (4.4 g, 87% yield) as a white solid; ¹H NMR (400 MHz, DMSO-d₆) δ ppm 5.01 (d, J=6.0 Hz, 1H), 3.92 (qd, J=7.0, 14.1 Hz, 1H), 3.79 (br s, 2H), 3.73 (br s, 2H), 2.41-2.35 (m, 2H), 1.95-1.88 (m, 2H), 1.36 (s, 9H).

### Example 631B. tert-butyl 6-((5-chloro-2-(methoxycarbonyl)thiophen-3-yl)oxy)-2-azaspiro[3.3]heptane-2-carboxylate

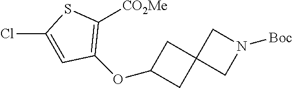 [SMILES: COC(=O)c1sc(Cl)cc1OC1CC2(C1)CN(C(=O)OC(C)(C)C)C2]

To a suspension of methyl 5-chloro-3-hydroxythiophene-2-carboxylate (2.0 g, 10.38 mmol), Example 631A (2.436 g, 11.42 mmol), triphenylphosphine (4.09 g, 15.58 mmol) in THF (25 mL), DIAD (3.03 mL, 15.58 mmol) was added. The mixture was stirred at rt for 3 h and diluted with ethyl acetate (60 mL) followed by the addition of water (100 mL). Ethyl acetate layer was separated, dried over Na₂SO₄, filtered and concentrated to afford yellow gummy solid, which was purified by silica gel chromatography (0-20% EtOAc in hexane) which afforded Example 631B (3.5 g, 87% yield) as a colorless thick liquid. MS: [M+H-Boc]⁺=288.0; ¹H NMR (400 MHz, CDCl₃) δ ppm 6.57 (s, 1H), 4.61 (quin, J=6.8 Hz, 1H), 3.96 (d, J=8.5 Hz, 4H), 3.83 (s, 3H), 2.76-2.67 (m, 2H), 2.54-2.41 (m, 2H), 1.46 (s, 9H).

### Example 631C. Methyl 3-((2-azaspiro[3.3]heptan-6-yl)oxy)-5-chlorothiophene-2-carboxylate

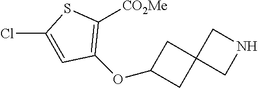 [SMILES: COC(=O)c1sc(Cl)cc1OC1CC2(CNC2)C1]

Reaction of Example 631B (1.0 g, 2.58 mmol) in b& (10 mL) and TFA (3.97 mL, 51.6 mmol) afforded Example 631C (600 mg, 58% yield) as a white solid. MS: [M+H]⁺=288.2 ¹H NMR (400 MHz, DMSO-d₆) δ ppm 7.17 (s, 1H), 4.70 (quin, J=6.8 Hz, 1H), 3.99 (br d, J=12.0 Hz, 4H), 3.72 (s, 3H), 2.82-2.74 (m, 2H), 2.34-2.26 (m, 2H).

### Example 631D. Methyl 5-chloro-3-((2-isopropyl-2-azaspiro[3.3]heptan-6-yl)oxy)thiophene-2-carboxylate

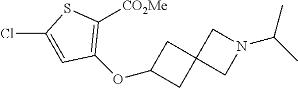 [SMILES: COC(=O)c1sc(Cl)cc1OC1CC2(C1)CN(C(C)C)C2]

Analogous to the procedure or the preparation of Example 438A, reaction of Example 631C (600 mg, 1.49 mmol) and acetone (0.164 mL, 2.24 mmol) afforded Example 631D (360 mg, 73% yield) as an off-white solid. MS: [M+H]⁺=330.2; ¹H NMR (400 MHz, DMSO-d₆) δ ppm 7.17 (s, 1H), 4.77-4.70 (m, 1H), 4.00 (br s, 4H), 3.72 (s, 3H), 2.91-2.66 (m, 2H), 2.39-2.25 (m, 2H), 1.07 (d, J=6.5 Hz, 6H).

### Example 356E. Lithium 5-chloro-3-((2-isopropyl-2-azaspiro[3.3]heptan-6-yl)oxy)thiophene-2-carboxylate

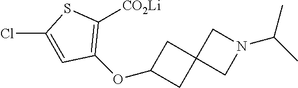

Analogous to the procedure for the preparation of Example 438B, reaction of Example 631D (360 mg, 1.09 mmol) and lithium hydroxide monohydrate (92 mg, 2.18 mmol) afforded Example 631E (350 mg, 100% yield) as a yellow solid. MS: [M+H]⁺=316.1; ¹H NMR (400 MHz, DMSO-d₆) δ ppm 6.77 (s, 1H), 4.67 (quin, J=7.0 Hz, 1H), 3.03 (d, J=17.5 Hz, 4H), 2.49-2.42 (m, 2H), 2.18-2.06 (m, 3H), 0.80 (d, J=6.0 Hz, 6H).

### Example 631F. (R)—N-(1-(4-amino-7-bromopyrrolo[2,1-f][1,2,4]triazin-5-yl)piperidin-3-yl)-5-chloro-3-((2-isopropyl-2-azaspiro[3.3]heptan-6-yl)oxy)thiophene-2-carboxamide

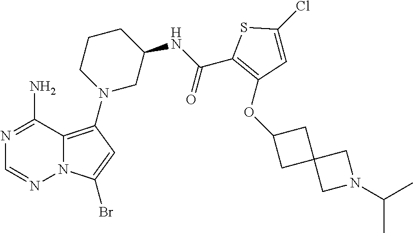 [SMILES: CC(C)N1CC2(CC(Oc3cc(Cl)sc3C(=O)N[C@@H]3CCCN(c4cc(Br)n5ncnc(N)c45)C3)C2)C1]

Analogous to the procedure for the preparation of Example 438C, reaction of (R)-5-(3-aminopiperidin-1-yl)-7-bromopyrrolo[2,1-f][1,2,4]triazin-4-amine (216 mg, 0.622 mmol) and Example 631E (200 mg, 0.622 mmol) afforded Example 631F (270 mg, 71% yield) as a yellow solid. MS: [M+H]⁺=608.2; ¹H NMR (400 MHz, DMSO-d₆) δ ppm 8.21-8.00 (m, 1H), 7.83 (s, 1H), 7.15 (s, 1H), 6.79 (s, 1H), 6.71-6.49 (m, 1H), 4.81 (t, J=6.5 Hz, 1H), 4.24-4.09 (m, 1H), 3.02-2.92 (m, 2H), 2.59 (br d, J=6.0 Hz, 2H), 2.44 (br s, 2H), 2.19-2.05 (m, 3H), 0.95 (br d, J=6.0 Hz, 9H), 0.75 (dd, J=2.5, 6.0 Hz, 6H).

### Example 631

To a solution of 631F (50 mg, 0.082 mmol) and (4-cyano-3-fluorophenyl)boronic acid (13.5 mg, 0.082 mmol) in THF (3 mL)-water (2 mL), were added potassium phosphate (0.082 mL, 0.164 mmol, 2M) and [1,1′-bis(di-tert butylphosphino)ferrocene]dichloropalladium(II) (3.2 mg, 4.9 μmol). The mixture was degassed with N₂ and heated at 70° C. for 2 h. After cooling, reaction mixture was filtrated and concentrated. Crude residue was purified by preparative HPLC to afford Example 631 as a white solid. LC-MS Method E: RT=1.760 min, [M+H]⁺=649.2; LC-MS Method F: RT=1.427 min, [M+H]⁺=649.3; ¹H NMR (400 MHz, DMSO-d₆) δ ppm 8.36 (dd, J=1.4, 11.9 Hz, 1H), 8.22 (dd, J=1.5, 8.3 Hz, 1H), 8.02-7.90 (m, 2H), 7.40 (s, 1H), 7.15 (s, 1H), 6.82 (s, 1H), 4.84-4.74 (m, 2H), 4.25-4.15 (m, 2H), 3.18-3.08 (m, 2H), 3.04-2.77 (m, 4H), 2.65-2.57 (m, 3H), 2.25-2.12 (m, 2H), 1.83 (br d, J=1.3 Hz, 3H), 1.63 (br s, 3H), 0.79 (s, 6H). ¹⁹F NMR (400 MHz, DMSO-d₆) δ ppm −108.483.

The following examples in Table 48 were prepared using the same procedure as shown in Example 631.

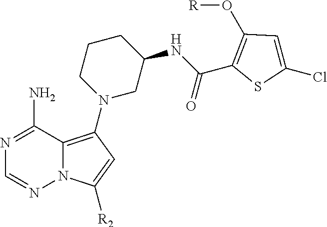

TABLE 48

| | | | | | HPLC | |
|---|---|---|---|---|---|---|
| | | | | LCMS | Method, | |
| Example | R | R₂ | Name | (M + H)⁺ | RT (min.) | ¹H NMR |
| 632 | 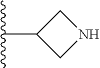 [SMILES: *C1CNC1] | 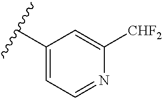 [SMILES: *c1ccnc(C(F)F)c1] | (R)-N-(1-(4-amino-7-(2- (difluoromethyl)pyridin-4- yl)pyrrolo[2,1- f][1,2,4]triazin-5- y1)piperidin-3-y1)-3- (azetidin-3-yloxy)-5- | 575.1 | E: 1.378 F: 1.092 | (400 MHz, DMSO-d6) δ ppm 8.69 (d, J = 5.1 Hz, 1H), 8.49 (s, 1H), 8.30 (br d, J = 5.4 Hz, 1H), 7.99 (s, 1H), 7.48 (s, 1H), 7.13-6.82 (m, 4H), 5.22-5.14 (m, 1H), 4.25-4.15 (m, 1H), 3.97-3.90 (m, 2H), 3.66-3.59 (m, 2H), 3.18 (s, |
| | | | chlorothiophene-2- | | | 2H), 3.03-2.73 (m, 4H), 1.89-1.52 (m, |
| | | | carboxamide | | | 4H); ¹⁹F NMR (400 MHz, CDCl₃) δ |
| | | | | | | ppm −115.316 |
| 633 | 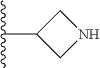 [SMILES: *C1CNC1] | 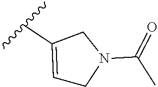 [SMILES: *C1=CCN(C(C)=O)C1] | (R)-N-(1-(7-(1-acetyl-2,5- dihydro-1H-pyrrol-3-yl)-4- aminopyrrolo[2,1- f][1,2,4]triazin-5- yl)piperidin-3-yl)-3- (azetidin-3-yloxy)-5- | 557.2 | E: 1.179 F: 0.883 | (400 MHz, DMSO-d₆) δ ppm 7.88 (s, 1H), 7.18-7.10 (m, 1H), 6.75 (dd, J = 19.6, 8.1 Hz, 3H), 6.52 (s, 2H), 5.25-5.13 (m. 1H), 4.71-4.63 (m, 1H), 4.48 (d, J = 6.0 Hz, 2H), 4.30- 4.11 (m, 4H), 3.96-3.79 (m, 2H), |
| | | | chlorothiophene-2- | | | 3.22-3.08 (m, 2H), 2.99-2.77 (m, |
| | | | carboxamide | | | 3H), 2.55 (s, 3H), 2.09-1.96 (m, 4H) |
| 634 | 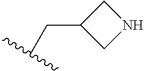 [SMILES: *CC1CNC1] | 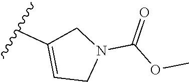 [SMILES: *C1=CCN(C(=O)OC)C1] | Methyl (R)-3-(4-amino-5- (3-(3-(azetidin-3~ ylmethoxy)-5- chlorothiophene-2- carboxamido)piperidin-1- y1)pyrrolo[2,1- | 587.2 | E: 1.351 F: 1.051 | (400 MHz, DMSO-d6) δ ppm 8.59 (br s, 2H), 7.87 (s, 1H), 7.35 (s, 1H), 7.22 (s, 1H), 7.09 (s, 1H), 6.97 (s, 1H), 6.75 (s, 2H), 4.54 (br dd, ) = 1.5, 11.5 Hz, 2H), 4.42 (br dd, J = 4.3, 6.8 Hz, 2H), 4.30 (br dd, J = 2.5, |
| | | | f][1,2,4]triazin-7-y1)-2,5- | | | 7.0 Hz, 4H), 4.06-3.93 (m, 3H), 3.89- |
| | | | dihydro-\H-pyrrole-1- | | | 3.74 (m, 3H), 3.66 (d, J = 4.5 Hz, |
| | | | carboxylate | | | 3H), 1.89-1.73 (m, 3H), 1.59-1.45 |
| | | | | | | (m, 2H) |
| 635 | 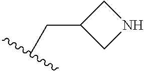 [SMILES: *CC1CNC1] | 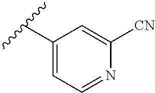 [SMILES: *c1ccnc(C#N)c1] | (R)-N-(1-(4-amino-7-(6- cyanopyridin-3- yl)pyrrolo[2,1- f\|[1,2,4]triazin-5- y1)piperidin-3-y1)-3- (azetidin-3-ylmethoxy)-5- | 564.1 | E: 1.370 F: 1.084 | (400 MHz, DMSO-d6) δ ppm 9.43 (d, J = 1.7 Hz, 1H), 8.83 (dd, J = 2.2, 8.3 Hz, 1H), 8.66-8.52 (m, 1H), 8.31-8.17 (m, 1H), 8.11 (d, J = 8.6 Hz, 1H), 7.97 (s, 1H), 7.42 (s, 1H), 7.35 (s, 1H), 4.46-4.39 (m, 2H), |
| | | | chlorothiophene-2- | | | 4.22-4.16 (m, 1H), 4.08-3.96 (m, |
| | | | carboxamide | | | 3H), 3.88-3.79 (m, 2H), 3.14-3.02 |
| | | | | | | (m, 2H), 2.98-2.80 (m, 3H), 1.92- |
| | | | | | | 1.73 (m, 3H), 1.56 (br d, J = 11.2 |
| | | | | | | Hz. 2H) |
| 636 | 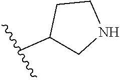 [SMILES: *C1CCNC1] | 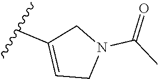 [SMILES: *C1=CCN(C(C)=O)C1] | N-((R)-1-(7-(1-acetyl-2,5- dihydro-1H-pyrrol-3-yl)-4- aminopyrrolo[2,1- f][1,2,4]triazin-5- yl)piperidin-3-y1)-5-chloro- | 571.1 | I: 7.029 J: 8.112 | (400 MHz, DMSO-d6) 8 ppm 8.05 (br s, 1H), 7.90-7.83 (m, 1H), 7.60- 7.50 (m, 1H), 7.35-7.29 (m, 1H), 6.82-6.69 (m, 3H), 5.11-4.98 (m, 1H), 4.75-4.64 (m, 1H), 4.55-4.42 |
| | | | 3-(pyrrolidin-3- | | | (m, 2H), 4.31-4.07 (m, 2H), 3.02- |
| | | | yloxy)thiophene-2- | | | 2.73 (m, 8H), 2.03 (br d, J = 16.4 |
| | | | carboxamide | | | Hz, 4H), 1.87-182 (m, 6H) |
| 637 | 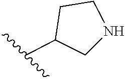 [SMILES: *C1CCNC1] | 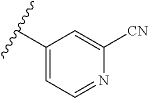 [SMILES: *c1ccnc(C#N)c1] | N-((R)-1-(4-amino-7-(6- cyanopyridin-3- yl)pyrrolo[2,1- f][1,2,4]triazin-5- yl)piperidin-3-y1)-5-chloro- 3-(pyrrolidin-3- | 564.1 | E: 1.423 F: 1.066 | (400 MHz, DMSO-d6) δ ppm 9.48- 9.37 (m, 1H), 8.80 (dd, J = 2.3. 8.5 Hz, 1H), 8.08 (dd, J = 0.8, 8.5 Hz, 1H), 7.98-7.89 (m, 1H), 7.55 (s, 1H), 7.44-7.37 (m, 1H), 7.31 (s, 1H), 6.96-6.74 (m, 2H), 5.08-4,99 (m, |
| | | | yloxy)thiophene-2- | | | 1H), 4.21-4.09 (m, 1H), 3.04-2.96 |
| | | | carboxamide | | | (m, 3H), 2.93-2.76 (m, 5H), 2.03- |
| | | | | | | 1.97 (m, 2H), 1.91-1.69 (m, 6H) |
| 638 | 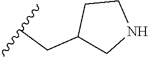 [SMILES: *CC1CCNC1] | 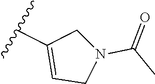 [SMILES: *C1=CCN(C(C)=O)C1] | N-((R)-1-(7-(1-acetyl-2,5- dihydro-1H-pyrrol-3-yl)-4- aminopyrrolo[2,1- f][1,2,4]triazin-5- y1)piperidin-3-yl)-5-chloro- | 585.2 | E: 1.178 F: 0.937 | (400 MHz, DMSO-d6) δ ppm 8.37- 8.14 (m, 1H), 7.90-7.85 (m, 1H), 7.34 (s, 2H), 6.78 (td, J = 2.0, 10.0 Hz, 1H), 6.73 (d, J = 11.0 Hz, 1H), 4.70 (br s, 1H), 4.52- 4.45 (m, 2H), |
| | | | 3-(pyrrolidin-3- | | | 4.29-4.25 (m, 1H), 4.19 (brd, J = 7.1 |
| | diastereomer 1 | | ylmethoxy)thiophone-2- | | | Hz. 4H), 3.14-2.94 (m, 5H), 2.89- |
| | | | carboxamide | | | 2.72 (m, 4H), 2.64-2.56 (m, 1H), |
| | | | | | | 2.04 (d. J = 16,4 Hz, 3H), 1.95-1.74 |
| | | | | | | (m, 4H), 1.54 (br s, 2H) |
| 639 | 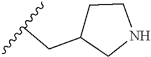 [SMILES: *CC1CCNC1] | 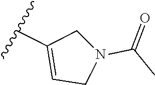 [SMILES: *C1=CCN(C(C)=O)C1] | N-((R)-1-(7-(1-acetyl-2,5- dihydro-1H-pyrrol-3-yl)-4- aminopyrrolo[2,1- f][1,2,4]triazin-5- yl)piperidin-3-y1)-5-chloro- 3-(pyrrolidin-3- | 585.2 | E: 1.186 F: 0.941 | (400 MHz, DMSO-d6) δ ppm 8.37- 8.14 (m, 1H),7.87 (s, 1H), 7.34 (s, 2H), 6.81-6.67 (m, 3H), 4.72-4.67 (m, 1H), 4.53-4.43 (m, 2H), 4.27 (br d, J = 0.7 Hz, 1H), 4.22-4.13 (m, 3H), 3.13-2.92 (m, 5H), 2.85-2.71 |
| | | | ylmethoxy)thiophene-2- | | | (m, 4H), 2.62-2.55 (m, 1H), 2.04 (d, |
| | diastereomer 2 | | carboxamide | | | J = 16,1 Hz, 4H), 1.96-1.77 (m, 4H), |
| | | | | | | 1.52 (br s, 1H) |
| 640 | 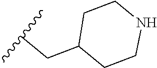 [SMILES: *CC1CCNCC1] | 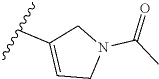 [SMILES: *C1=CCN(C(C)=O)C1] | (R)-N-(1-(7-(1-acetyl-2,5- dihydro-1H-pyrrol-3-yl)-4- aminopyrrolo[2,1- f][1,2,4]triazin-5- y1)piperidin-3-y1)-5-chloro- | 599.2 | E: 1.207 F: 0.975 | (400 MHz, DMSO-d6) δ ppm 8.05- 8.07 (m, 1H), 7.91-7.84 (m, 1H), 7.41-7.32 (m, 2H), 6.83-6.68 (m, 3H), 4.72-4.43 (m, 4H), 4.31-4.02 (m, 5H), 3.59-3.42 (m, 5H), 3.24- |
| | | | 3-(piperidin-4- | | | 3.11 (m, 2H), 3.10-2.75 (m, 4H), |
| | | | ylmethoxy)thiophene~2- | | | 2.09-1.96 (m, 3H), 1.86-1.68 (m, |
| | | | carboxamide | | | 4H), 1.95-1.63 (m, 1H) |
| 641 | 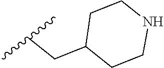 [SMILES: *CC1CCNCC1] | 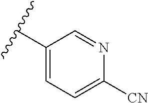 [SMILES: *c1ccc(C#N)nc1] | (R)-N-(1-(4-amino-7-(6~ cyanopyridin-3- y1)pyrrolo[2,1- f][1,2,4]triazin-5- y1)piperidin-3-yl)-5-chloro- 3-(piperidin-4- | 592.2 | G: 5.015 K: 6.197 | (400 MHz, DMSO-d6) δ ppm 9.52- 9.37 (m, 1H), 8.90-8.70 (m, 1H), 8.32-8.16 (m, 2H), 8.14-8.02 (m, 1H), 8.01-7.91 (m, 1H), 7.57-7.27 (m, 3H), 4.24-3.99 (m, 4H), 3.03- 2.74 (m, SH), 1.91-1.70 (m, 7H), |
| | | | ylmethoxy)thiophene-2- | | | 1.67-1.52 (m, 3H), 1.17-1.00 (m, 2H) |
| | | | carboxamide | | | |
| 642 | 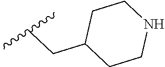 [SMILES: *CC1CCNCC1] | 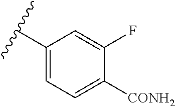 [SMILES: *c1ccc(C(N)=O)c(F)c1] | (R)-N-(1-(4-amino-7-(4- carbamoyl-3- fluorophenyl)pyrrolo[2,1- f][1,2,4]triazin-5- y1)piperidin-3-y1)-5-chloro- 3-(piperidin-4- ylmethoxy )thiophene-2- | 627.2 | E: 1.256 F: 1.003 | (400 MHz, DMSO-d6) δ ppm 8.16- 8.10 (m, 1H), 8.00 (dd, J = 1.6, 8.2 Hz, 1H), 7.93 (s, 1H), 7.75 (t, J = 8.1 Hz. 1H), 7.68-7.57 (m, 2H), 7.45-7.16 (m, 2H), 6.79 (s, 1H), 4.18 (br d, J = 3.9 Hz, 1H), 4.13-4.05 (m, 2H), 3.18 (br s, 1H), 3.02-2.83 (m, 5H), 2.45- |
| | | | carboxamide | | | 2.33 (m, 3H), 1.97-1.76 (m, 6H), |
| | | | | | | 1.73-1.61 (m, 2H), 1.21-1.04 (m, |
| | | | | | | 2H); ¹⁹F NMR (400 MHz, CDCl₃) |
| | | | | | | δ = −112.962 |
| 643 | 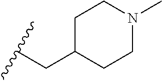 [SMILES: *CC1CCN(C)CC1] | 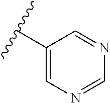 [SMILES: *c1cncnc1] | (R)-N-(1-(4-amino-7- (pyrimidin-5- yl)pyrrolo[2,1- f][1,2,4]triazin-5- y1)piperidin-3-y1)-5-chloro- 3-((1-methylpiperidin-4- yl)methoxy)thiophene-2- | 582.2 | E: 1.202 F: 0.954 | (400 MHz, DMSO-d6) δ ppm 9.48 (s, 3H), 9.10 (s, 1H), 7.94 (s, 1H), 7.37 (s, 3H), 7.31 (s, 1H), 4.22-4.07 (m, 4H), 3.21-3.13 (m, 3H), 3.01- 2.81 (m, 4H), 2.00-1.71 (m, 9H), 1.40 (br d, J = 9.3 Hz, 3H) |
| | | | carboxamide | | | |
| 644 | 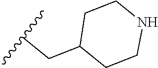 [SMILES: *CC1CCNCC1] | 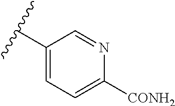 [SMILES: *c1ccc(C(N)=O)nc1] | (R)-5-(4-amino-5-(3-(5- chloro-3-(piperidin-4- ylmethoxy)thiophene-2- carboxamido)piperidin-1- y1)pyrrolo[2,1- f][1,2,4]triazin-7- y1)picolinamide | 610.2 | E: 1.155 F: 0.963 | (400 MHz, DMSO-d6) δ ppm 9.30 (d, J = 1.5 Hz, 1H), 8.68 (dd, J = 2.3, 8.2 Hz, 1H), 8.10 (d, J = 8.3 Hz, 3H), 7.93 (s, 1H), 7.65 (br s, 1H), 7.37 (s, 1H), 7.29 (s, 1H), 4.24-4.19 (m, 1H), 4.09 (br t, J = 6.5 Hz, 2H), 3.02-2.83 (m, 6H), 2.34 (s, 2H), |
| | | | | | | 1.93-1.77 (m, 7H), 1.72-1.59 (m, |
| | | | | | | 3H), 1.23-1.10 (m, 2H) |

The following examples in Table 49 were prepared using the same procedures as shown in Example 479.

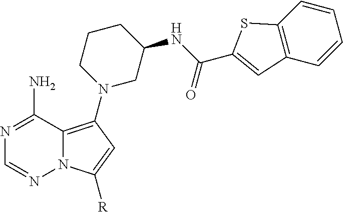

TABLE 49

| | | | | HPLC | |
|---|---|---|---|---|---|
| | | | LCMS | Method, | |
| Example | R | Name | (M + H)⁺ | RT (min.) | ¹H NMR |
| 645 | 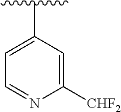 [SMILES: *c1ccnc(C(F)F)c1] | (R)-N-(1-(4-amino-7-(2- (difluoromethyl)pyridin-4- y1)pyrrolo[2,1- f][1,2,4]triazin-5- y1)piperidin-3- yl)benzo[b]thiophene-2- carboxamide | 520.1 | A. 1.845 B; 1.555 | (400 MHz, DMSO-d₆) δ ppm 8.74-8.62 (m, 2H), 8.50 (s, 1H), 8.32 (d, J = 5.4 Hz, 1H), 8.18 (s, 2H), 8.05- 7.98 (m, 2H), 7.98-7.90 (m, 1H), 7.50-7.40 (m, 3H), 7.14-6.76 (m, 2H), 4.25-4.16 (m, 1H), 3.29-3.22 (m, 1H), 3.12-3.01 (m, 1H), 2.87-2.70 (m, 2H), 2.03-1.79 (m, 3H), 1.68-1.48 (m, 1H) |
| 646 | 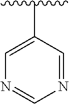 [SMILES: *c1cncnc1] | (R)-N-(1-(4-amino-7- (pyrimidin-5-yl)pyrrolo[2,1- f][1,2,4]triazin-5- yl)piperidin-3- yl)benzo[b]thiophene-2- carboxamide. | 471.2 | A. 1.606 B. 1.315 | (400 MHz, DMSO-d6) 8 ppm 9.48 (s, 2H), 9.09 (s, 1H), 8.70 (d, J = 7.8 Hz, 1H), 8.21-8.11 (m, 1H), 8.04-7.89 (m, 3H), 7.49-7.43 (m, 2H), 7.33 (s, 1H), 7.02 (s, 2H), 4.25-4.18 (m, 1H), 3.28-3.25 (m. 1H), 3.11-3.02 (m, 1H), 2.82-2.72 (m, 2H), 1.99-1.82 (m, 3H), 1.64-1.54 (m, 1H). |

The following examples in Table 50 were prepared by coupling Intermediate 34 (R)-5-(4-amino-5-(3-aminopiperidin-1-yl)pyrrolo[2,1-f][1,2,4]triazin-7-yl)picolinonitrile with respective acids.

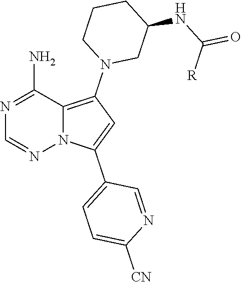

TABLE 50

| | | | | HPLC | |
|---|---|---|---|---|---|
| | | | LCMS | Method, | |
| Example | R | Name | (M + H)⁺ | RT (min.) | ¹H NMR |
| 647 | 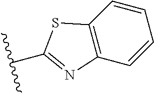 [SMILES: *c1nc2ccccc2s1] | (R)-N-(1-(4-amino-7- (6-cyanopyridin-3- yl)pyrrolo[2,1- f][1,2,4]triazin-5- yl)piperidin-3- yl)benzo[d]thiazole-2- | 496.2 | A. 1.864 B. 1.510 | (400 MHz, DMSO-d₆) δ ppm 9.45 (d, J = 2.0 Hz, 1H), 9.17 (d, J = 7.8 Hz, 1H), 8.86-8.78 (m, 1H), 8.23 (d, J = 7.8 Hz, 1H), 8.17 (d, J = 7.6 Hz, 1H), 8.09 (d, J = 8.3 Hz, 1H), 7.96 (s, 1H), 7.68-7.51 (m, 2H), 7.42 (d, 2H), 4.26 (d, 2H), 3.10-3.03 (m, 1H), 2.90-2.84 (m, 1H), 2.75 (br d, J = 2.2 Hz, 2H), 1.97-1.81 (m, 3H), 1.77-1.68 (m, 1H) |
| | | carboxamide | | | |
| 648 | 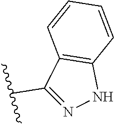 [SMILES: *c1n[nH]c2ccccc12] | (R)-N-(1-(4-amino-7- (6-cyanopyridin-3- yl)pyrrolo[2,1- f][1,2,4]triazin-5- yl)piperidin-3-y1)-1H- indazole-3- carboxamide. | 479.2 | A. 1.556 B. 1.240 | (400 MHz, DMSO-d₆) δ ppm 9.50-9.43 (m, 1H), 9.32 (d, J = 7.8 Hz, 1H), 8.86-8.78 (m, 1H), 8.40-8.14 (m, 1H), 8.10 (dd, J = 0.7, 8.3 Hz, 1H), 7.98 (s, 1H), 7.94-7.83 (m, 2H), 7.61-7.46 (m, 2H), 7.42 (s, 1H), 7.26-6.97 (m, 1H), 4.33-4.26 (m, 2H), 3.25-3.20 (m, 1H), 3.09-2.99 (m, 1H), 2.92-2.70 (m, 2H), 1.98-1.79 (m, 3H), 1.75~1.60 (m, 1H) |
| 649 | 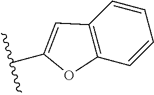 [SMILES: *c1cc2ccccc2o1] | (R)-N-(1-(4-amino-7- (6-cyanopyridin-3- yl)pyrrolo[2,1- f\|[1,2,4]triazin-5- y1)piperidin-3- yl)benzofuran-2- | 479.2 | B.1.412 | (400 MHz, DMSO-d₆) δ ppm 9.45 (d, J = 1.5 Hz, 1H), 8.87-8.77 (m, 1H), 8.67 (d, J = 7.8 Hz, 1H), 8.30-8.15 (m, 1H), 8.09 (d, J = 8.3 Hz, 1H), 7.96 (s, 1H), 7.78 (d, J = 7.6 Hz. 1H), 7.67 (d, ) = 8.6 Hz, 1H), 7.58 (s, 1H), 7.47 (t, J = 7.8 Hz, 1H), 7.42 (s, 1H), 7.36-7.28 (m, 1H), 7.05 (s, 1H), 4.31-4.20 (m, 1H), 3.27-3.23 (m, 1H), 3.12-3.01 (m, 1H), |
| | | carboxamide | | | 2.87-2.70 (m, 2H), 2.00-1.79 (m, 3H), 1,71-1.59 (m, 1H) |
| 650 | 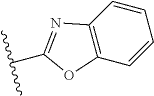 [SMILES: *c1nc2ccccc2o1] | (R)-N-(1-(4-amino-7- (6-cyanopyridin-3- yl)pyrrolo[2,1- f][1,2,4]triazin-5- yl)piperidin-3- yl)benzo[d]oxazole-2- | 480.2 | A. 1.645 B. 1.316 | (400 MHz, DMSO-d₆) δ ppm 9.50-9.43 (m, 1H), 9.32 (d, J = 7.8 Hz, 1H), 8.86-8.78 (m, 1H), 8.40-8.14 (m, 1H), 8.10 (dd, J = 0.7, 8.3 Hz, 1H), 7.98 (s, 1H), 7.94-7.83 (m, 2H), 7.61-7.46 (m, 2H), 7.42 (s, 1H), 7.26-6.97 (m, 1H), 4.33- 4.26 (m, 1H), 3.25-3.20 (m, 1H), 3.09-2.99 (m, 1H), 2.92- 2.70 (m, 2H), 1.98-1.79 (m, 3H), 1.75-1.60 (m, 1H) |
| | | carboxamide. | | | |

### Example 651

### Preparation of ter-butyl (R)-4-((2-((1-(4-amino-7-(1-(methoxycarbonyl)-2,5-dihydro-1H-pyrrol-3-yl)pyrrolo[2,1-f][1,2,4]triazin-5-yl)piperidin-3-yl)carbamoyl)benzo[b]thiophen-3-yl)oxy)piperidine-1-carboxylate

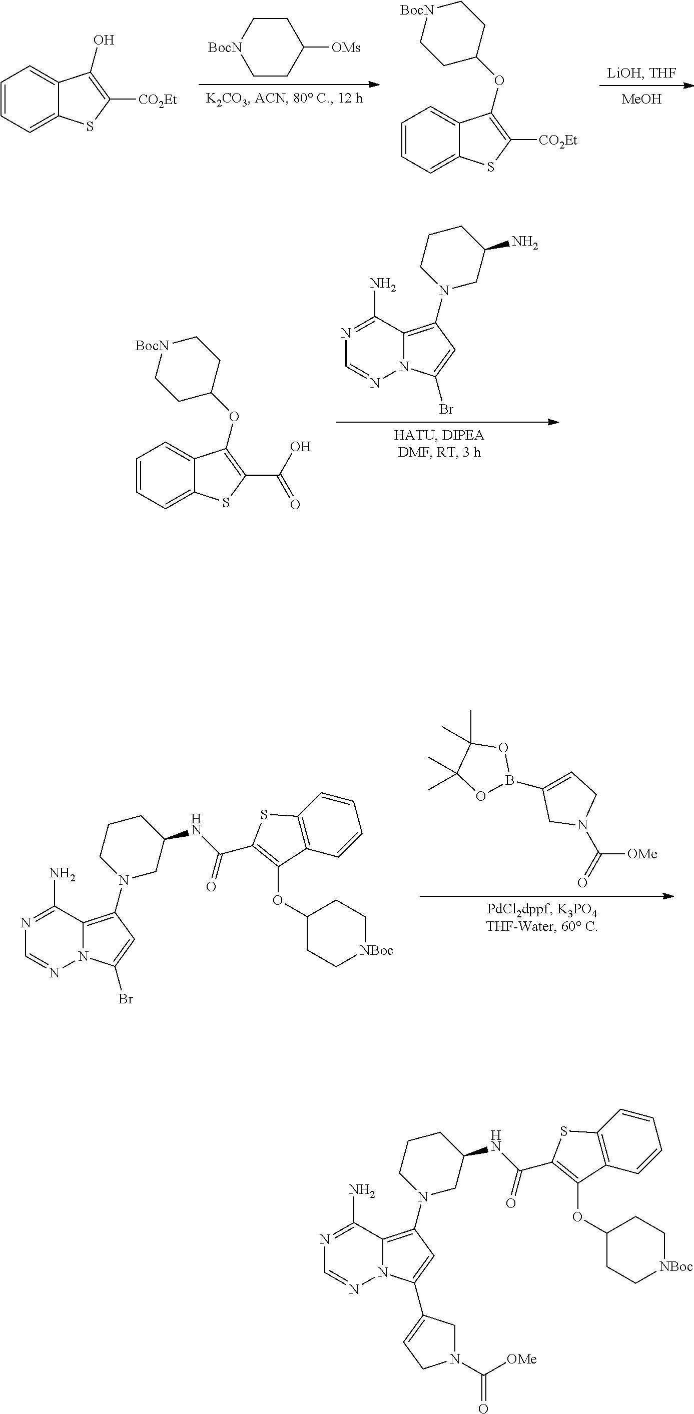

### Example 651A. tert-butyl 4-((2-(methoxycarbonyl) benzo[b]thiophen-3-yl)oxy)piperidine-1-carboxylate

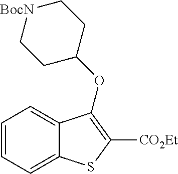 [SMILES: CCOC(=O)c1sc2ccccc2c1OC1CCN(C(=O)OC(C)(C)C)CC1]

To a stirred solution of ethyl 3-hydroxybenzo[b]thiophene-2-carboxylate (400 mg, 1.800 mmol) in DMF (10 mL), tert-butyl 4-((methylsulfonyl)oxy) piperidine-1-carboxylate (754 mg, 2.70 mmol) and K₂CO₃ (497 mg, 3.60 mmol) were added. Reaction mixture was allowed to stir at 80° C. for 12 h. After cooling, it was diluted with ethyl acetate (60 ml), followed by the addition of water (50 mL). Ethyl acetate layer was separated, dried over Na₂SO₄ and concentrated. The residue was purified by flash chromatography (ethyl acetate and pet-ether as eluents) to afford Example 651A. (500 mg, 1.233 mmol, 68.5% yield). MS: [M−56+H]⁺=351.3; ¹H NMR (400 MHz, DMSO-d₆) δ ppm 8.01-7.95 (m, 1H), 7.93 (d, J=8.0 Hz, 1H), 7.60-7.53 (m, 1H), 7.53-7.46 (m, 1H), 4.69 (td, J=4.5, 8.7 Hz, 1H), 4.33 (q, J=7.4 Hz, 2H), 3.88-3.75 (m, 3H), 3.05 (br s, 1H), 1.96-1.83 (m, 2H), 1.80-1.53 (m, 2H), 1.42 (s, 9H), 1.38-1.26 (m, 3H).

### Example 651B. Preparation of 3-((1-(tert-butoxycarbonyl) piperidin-4-yl)oxy)benzo[b]thiophene-2-carboxylic acid

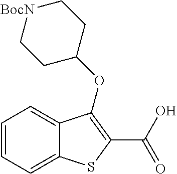 [SMILES: CC(C)(C)OC(=O)N1CCC(Oc2c(C(=O)O)sc3ccccc23)CC1]

Example 651A (300 mg, 0.740 mmol) was dissolved in a mixture of THF (5 mL), MeOH (2 mL) and H₂O (3 mL). Then, LiOH monohydrate (17.7 mg, 0.74 mmol) was added and stirred at rt for 12 h. The reaction mixture was evaporated to dryness and suspended in water (20 mL). It was cooled to 10° C. and acidified with 1N HCl till the pH 2. The organic compound was extracted with ethyl acetate (15 mL×3) and the combined organic layer was washed with water, brine dried on sodium sulphate, filtered, and evaporated to afford Example 651B. (200 mg, 0.530 mmol, 72% yield). MS. [M−56+H]⁺=322.2; ¹H NMR (400 MHz, DMSO-d₆) δ ppm 7.96 (d, J=8.0 Hz, 1H), 7.90 (d, J=8.0 Hz, 1H), 7.57-7.50 (m, 1H), 7.50-7.45 (m, 1H), 4.70 (td, J=4.4, 8.3 Hz, 1H), 3.82-3.74 (m, 2H), 3.27-3.01 (m, 3H), 1.95-1.87 (m, 1H), 1.73-1.55 (m, 2H), 1.44-1.38 (m, 9H).

### Example 651C. tert-butyl (R)-4-((2-((1-(4-amino-7-bromopyrrolo[2,1-f][1,2,4]triazin-5-yl)piperidin-3 yl)carbamoyl)benzo[b]thiophen-3-yl)oxy)piperidine-1-carboxylate

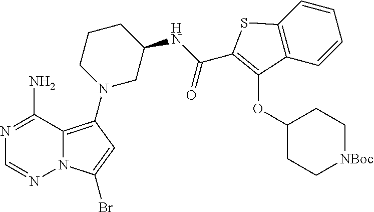 [SMILES: CC(C)(C)OC(=O)N1CCC(Oc2c(C(=O)N[C@@H]3CCCN(c4cc(Br)n5ncnc(N)c45)C3)sc3ccccc23)CC1]

Analogous to the procedure for the preparation of Example 276C, reaction of (R)-5-(3-aminopiperidin-1-yl)-7-bromopyrrolo[2,1-f][1,2,4]triazin-4-amine (500 mg, 1.18 mmol) with Example 651B to afford Example 651C (300 mg, 38% yield). MS. [M+H]⁺=670.2.

### Example 651

Analogous to the procedure for the preparation of Example 276, reaction of Example 651C (200 mg, 0.298 mmol) with Intermediate 28, afforded Example 651 (150 mg, 70% yield). LC-MS Method E: RT=2.181 min, [M+H]⁺=717.2; LC-MS Method F: RT=1.868 min; ¹H NMR (400 MHz, DMSO-d₆) δ ppm 8.01-7.90 (m, 3H), 7.87 (s, 2H), 7.49 (dquin, J=1.2, 7.3 Hz, 2H), 6.77-6.69 (m, 2H), 4.70-4.60 (m, 1H), 4.58-4.51 (m, 2H), 4.33-4.18 (m, 3H), 3.87-3.72 (m, 2H), 3.65 (d, J=3.2 Hz, 4H), 3.25-3.20 (m, 1H), 3.00-2.70 (m, 5H), 2.07-1.76 (m, 4H), 1.66-1.48 (m, 4H), 1.34 (s, 9H).

### Example 652

### Preparation of methyl (R)-3-(4-amino-5-(3-(3-(piperidin-4-yloxy)benzo[b]thiophene-2-carboxamido)piperidin-1-yl)pyrrolo[2,1-f][1,2,4]triazin-7-yl)-2,5-dihydro-1H-pyrrole-1-carboxylate

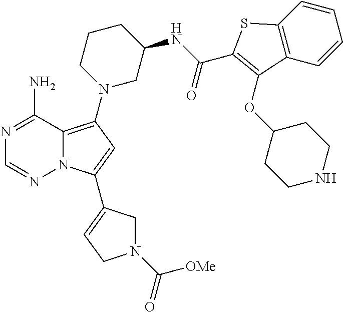 [SMILES: COC(=O)N1CC=C(c2cc(N3CCC[C@@H](NC(=O)c4sc5ccccc5c4OC4CCNCC4)C3)c3c(N)ncnn23)C1]

To a stirred solution of Example 651 (100 mg, 0.139 mmol) in DCM (5 mL), was added TFA (1 mL, 0.139 mmol) at rt and the reaction mass was allowed to stir for 2 h. Volatiles were removed under vacuo to afford Example 652 (80 mg, 93% yield). LC-MS Method E: RT=1.472 min, [M+H]+=617.3; LC-MS Method F: RT=1.188 min; 1H NMR (400 MHz, DMSO-d6) δ ppm 8.01-7.90 (m, 3H), 7.87 (s, 2H), 7.49 (dquin, J=1.2, 7.3 Hz, 2H), 6.77-6.69 (m, 2H), 4.70-4.60 (m, 1H), 4.58-4.51 (m, 2H), 4.33-4.18 (m, 3H), 3.87-3.72 (m, 3H), 3.65 (d, J=3.2 Hz, 4H), 3.25-3.20 (m, 1H), 3.00-2.70 (m, 5H), 2.07-1.76 (m, 4H), 1.66-1.48 (m, 4H)

### Example 653

### Preparation of tert-butyl (R)-4-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)benzo[b]thiophen-3-yl)oxy)piperidine-1-carboxylate

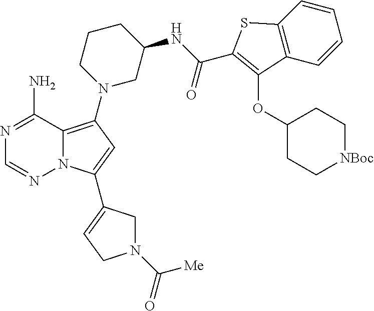 [SMILES: CC(=O)N1CC=C(c2cc(N3CCC[C@@H](NC(=O)c4sc5ccccc5c4OC4CCN(C(=O)OC(C)(C)C)CC4)C3)c3c(N)ncnn23)C1]

Analogous to the procedure for the preparation of Example 651, reaction of Example 651C and Intermediate 27 afforded Example 653 (3.9 mg, 4% yield). LC-MS Method E: RT=1.935 min, [M+H]⁺=701.3; LC-MS Method F: RT=1.681 min, [M+H]⁺=701.2; ¹H NMR (400 MHz, DMSO-d₆) ppm 8.02-7.89 (m, 3H), 7.88 (d, J=2.4 Hz, 1H), 7.56-7.43 (m, 2H), 6.82-6.67 (m, 2H), 4.72-4.61 (m, 2H), 4.55-4.41 (m, 2H), 4.30-4.15 (m, 2H), 3.88-3.67 (m, 2H), 3.26-3.13 (m, 3H), 3.02-2.75 (m, 5H), 2.07-1.73 (m, 7H), 1.67-1.45 (m, 4H), 1.34 (d, J=2.9 Hz, 9H).

### Example 654

### Preparation of tert-butyl (R)-4-((2-((1-(4-amino-7-(6-cyanopyridin-3-yl)pyrrolo[2,1-f][1,2,4]triazin-5-yl)piperidin-3-yl)carbamoyl)benzo[b]thiophen-3-yl)oxy)piperidine-1-carboxylate

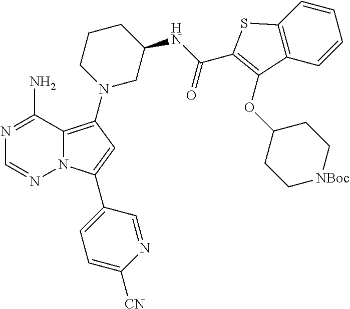 [SMILES: CC(C)(C)OC(=O)N1CCC(Oc2c(C(=O)N[C@@H]3CCCN(c4cc(-c5ccc(C#N)nc5)n5ncnc(N)c45)C3)sc3ccccc23)CC1]

Analogous to the procedure for the preparation of Example 651, reaction of Example 651C and 5-(4,4,5,5-tetramethyl-1,3,2-dioxaborolan-2-yl) picolinonitrile afforded Example 654 (9.6 mg, 9% yield). LC-MS Method E: RT=2.234 min, [M+H]⁺=694.2; LC-MS Method F: RT=1.957 min, [M+H]⁺=694.3; ¹H NMR (400 MHz, DMSO-d₆) δ ppm 9.45 (s, 1H), 8.83 (dd, J=2.2, 8.3 Hz, 1H), 8.21 (br d, J=5.6 Hz, 1H), 8.10 (d, J=8.3 Hz, 1H), 8.04-7.86 (m, 4H), 7.57-7.44 (m, 2H), 7.44-7.28 (m, 1H), 6.97 (br s, 1H), 4.69 (br s, 1H), 4.28 (dt, J=4.2, 6.4 Hz, 1H), 3.86-3.68 (m, 2H), 3.31-3.24 (m, 1H), 3.07-2.74 (m, 5H), 2.08-1.77 (m, 5H), 1.65-1.49 (m, 3H), 1.29 (br s, 9H).

### Example 655

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-3-(piperidin-4-yloxy)benzo[b]thiophene-2-carboxamide

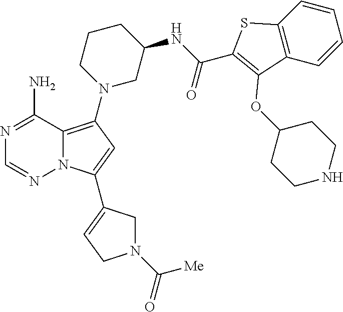 [SMILES: CC(=O)N1CC=C(c2cc(N3CCC[C@@H](NC(=O)c4sc5ccccc5c4OC4CCNCC4)C3)c3c(N)ncnn23)C1]

Analogous to the procedure for the preparation of Example 652, reaction of Example 653 and TFA afforded Example 655 (0.8 mg, 2% yield). LC-MS Method E: RT=1.283 min, [M+H]⁺=601.2; LC-MS Method F: RT=1.045 min, [M+H]⁺=601.3; ¹H NMR (400 MHz, DMSO-d₆) δ ppm 8.01-7.85 (m, 6H), 7.55-7.45 (m, 3H), 6.82-6.63 (m, 2H), 4.72-4.65 (m, 1H), 4.61-4.43 (m, 4H), 4.27-4.18 (m, 2H), 3.08-3.01 (m, 4H), 2.83-2.74 (m, 2H), 2.62-2.56 (m, 2H), 2.09-1.80 (m, 7H), 1.72-1.57 (m, 3H).

### Example 656

### Preparation of (R)—N-(1-(4-amino-7-(6-cyanopyridin-3-yl)pyrrolo[2,1-f][1,2,4]triazin-5-yl)piperidin-3-yl)-3-(piperidin-4-yloxy)benzo[b]thiophene-2-carboxamide

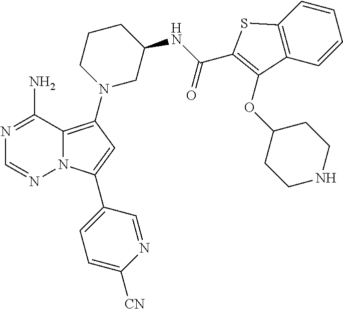 [SMILES: N#Cc1ccc(-c2cc(N3CCC[C@@H](NC(=O)c4sc5ccccc5c4OC4CCNCC4)C3)c3c(N)ncnn23)cn1]

Analogous to the procedure for the preparation of Example 652, reaction of Example 654 and TFA afforded Example 656 (17.3 mg, 47% yield). LC-MS Method E: RT=1.501 min. [M+H]⁺=594.2; LC-MS Method F: RT=1.180 min, [M+H]⁺=594.2; ¹H NMR (400 MHz, DMSO-d₆) δ ppm 9.48-9.18 (m, 1H), 8.83 (dd, J=2.3, 8.4 Hz, 1H), 8.59-8.16 (m, 3H), 8.14-7.95 (m, 4H), 7.91 (d, J=6.8 Hz, 1H), 7.58-7.45 (m, 2H), 7.42 (s, 1H), 7.09-6.93 (m, 1H), 4.74-4.63 (m, 1H), 4.30-4.20 (m, 1H), 3.35-3.25 (m, 4H), 3.11-2.91 (m, 3H), 2.86-2.71 (m, 2H), 2.27-2.18 (m, 1H), 2.16-2.06 (m, 1H), 2.03-1.79 (m, 3H), 1.71-1.54 (m, 1H).

### Example 657

### Preparation of (R)—N-(1-(4-amino-7-(pyrimidin-5-yl)pyrrolo[2,1-f][1,2,4]triazin-5-yl)piperidin-3-yl)-3-(piperidin-4-yloxy)benzo[b]thiophene-2-carboxamide

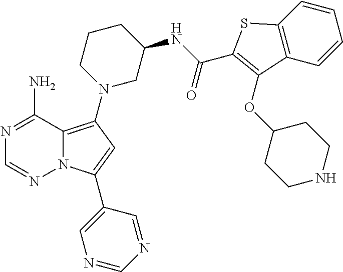 [SMILES: Nc1ncnn2c(-c3cncnc3)cc(N3CCC[C@@H](NC(=O)c4sc5ccccc5c4OC4CCNCC4)C3)c12]

### Example 657A. tert-butyl (R)-4-((2-((1-(4-amino-7-(pyrimidin-5-yl)pyrrolo[2,1-f][1,2,4]triazin-5-yl)piperidin-3-yl)carbamoyl)benzo[b]thiophen-3-yl)oxy)piperidine-1-carboxylate

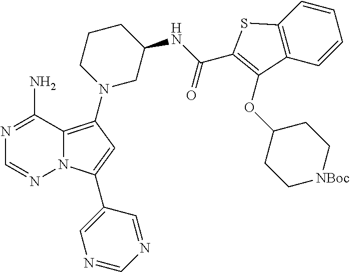 [SMILES: CC(C)(C)OC(=O)N1CCC(Oc2c(C(=O)N[C@@H]3CCCN(c4cc(-c5cncnc5)n5ncnc(N)c45)C3)sc3ccccc23)CC1]

Analogous to the procedure for the preparation of Example 651, reaction of Example 651C and 5-(4,4,5,5-tetramethyl-1,3,2-dioxaborolan-2-yl)pyrimidine (32.0 mg, 0.155 mmol) afforded Example 657A (40 mg, 50% yield). LC-MS Method E: RT=2.234 min, [M+H]⁺=694.2; LC-MS Method F: RT=1.957 min.

### Example 657

Analogous to the procedure for the preparation of Example 656, reaction of Example 657A and TFA afforded Example 657 (14.4 mg, 17% yield). LC-MS Method E: RT=1.290 min, [M+H]⁺=570.2; LC-MS Method F: RT=1.078 min, [M+H]⁺=570.3; ¹H NMR (400 MHz, DMSO-d₆) δ ppm 9.48 (s, 2H), 9.09 (s, 2H), 7.98 (br d, J=6.7 Hz, 2H), 7.91 (br d, J=7.3 Hz, 1H), 7.54-7.44 (m, 3H), 7.32 (s, 1H), 4.61-4.51 (m, 1H), 4.31-4.22 (m, 4H), 3.06-2.77 (m, 4H), 1.91-1.80 (m, 7H), 1.63 (br s, 4H).

### Example 658

### Preparation of methyl (R)-3-(4-amino-5-(3-(3-((1-methylpiperidin-4-yl) oxy) benzo[b]thiophene-2-carboxamido) piperidin-1-yl) pyrrolo[2,1-f][1,2,4] triazin-7-yl)-2,5-dihydro-1H-pyrrole-1-carboxylate

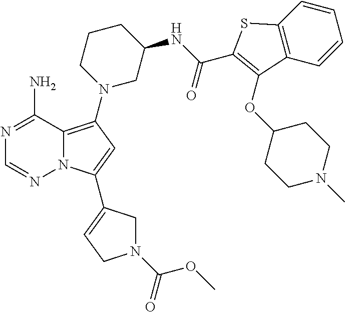 [SMILES: COC(=O)N1CC=C(c2cc(N3CCC[C@@H](NC(=O)c4sc5ccccc5c4OC4CCN(C)CC4)C3)c3c(N)ncnn23)C1]

To a stirred solution of Example 652 (50 mg, 0.081 mmol) in MeOH (2 mL), formaldehyde (2.233 μl, 0.081 mmol) and sodium cyanoborohydride (5.1 mg, 0.081 mmol) were added and the mixture was allowed to stir at rt for 3 h. The reaction mixture was concentrated and purified by preparative HPLC to afford Example 658 (8.7 mg, 16% yield). LC-MS Method E: RT=1.773 min, [M+H]+=631.2; LC-MS Method F: RT=1.199 min, [M+H]+=631.3; 1H NMR (400 MHz, DMSO-d₆) δ ppm 8.02-7.93 (m, 2H), 7.93-7.89 (m, 1H), 7.87 (s, 1H), 7.54-7.43 (m, 2H), 6.77-6.69 (m, 2H), 4.58-4.50 (m, 2H), 4.48 (br d, J=8.6 Hz, 1H), 4.33-4.26 (m, 2H), 4.23 (br dd, J=2.3, 5.0 Hz, 1H), 3.91 (s, 1H), 3.66 (d, J=3.2 Hz, 3H), 3.27 (br s, 1H), 3.02-2.88 (m, 2H), 2.84-2.68 (m, 3H), 2.22-2.12 (m, 3H), 2.08-1.93 (m, 4H), 1.89-1.71 (m, 4H), 1.58 (br s, 3H).

### Example 659

### Preparation of methyl (R)-3-(4-amino-5-(3-(3-((1-isopropylpiperidin-4-yl)oxy)benzo[b]thiophene-2-carboxamido)piperidin-1-yl)pyrrolo[2,1-f][1,2,4]triazin-7-yl)-2,5-dihydro-1H-pyrrole-1-carboxylate

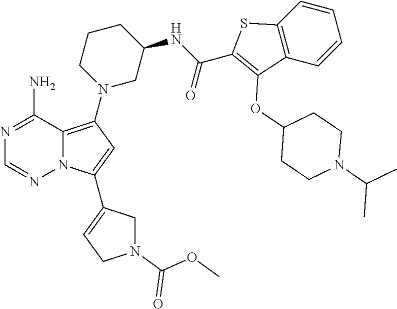 [SMILES: COC(=O)N1CC=C(c2cc(N3CCC[C@@H](NC(=O)c4sc5ccccc5c4OC4CCN(C(C)C)CC4)C3)c3c(N)ncnn23)C1]

Analogous to the procedure for the preparation of Example 658, reaction of Example 652 and acetone afforded Example 659 (7.3 mg, 16% yield). LC-MS Method E: RT=1.846 min, [M+H]⁺=659.3; LC-MS Method F: RT=1.259 min, [M+H]⁺=659.4; ¹H NMR (400 MHz, DMSO-d₆) δ ppm 9.10 (br s, 1H), 8.02-7.96 (m, 1H), 7.94-7.85 (m, 3H), 7.56-7.43 (m, 2H), 6.74 (s, 2H), 4.71-4.62 (m, 1H), 4.58-4.47 (m, 2H), 4.36-4.25 (m, 2H), 4.21 (br dd, J=3.8, 5.0 Hz, 1H), 3.65 (d, J=3.5 Hz, 3H), 3.47 (br d, J=1.8 Hz, 1H), 3.28 (br dd, J=2.3, 8.3 Hz, 2H), 3.09-2.92 (m, 1H), 2.82-2.69 (m, 2H), 2.36 (br dd, J=2.3, 13.3 Hz, 1H), 2.29-2.13 (m, 3H), 2.06-1.77 (m, 3H), 1.60 (m, 5H), 1.26-1.15 (m, 7H).

### Example 660

### Preparation of methyl (R)-3-(4-amino-5-(3-(3-((1-(2-hydroxyethyl)piperidin-4-yl)oxy)benzo[b]thiophene-2-carboxamido)piperidin-1-yl)pyrrolo[2,1-f][1,2,4]triazin-7-yl)-2,5-dihydro-1H-pyrrole-1-carboxylate

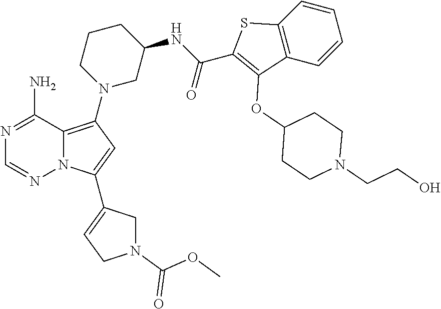 [SMILES: COC(=O)N1CC=C(c2cc(N3CCC[C@@H](NC(=O)c4sc5ccccc5c4OC4CCN(CCO)CC4)C3)c3c(N)ncnn23)C1]

To a stirred solution of Example 652 (30 mg, 0.049 mmol) in DCM (2 mL), 2-bromoethan-1-ol (6.08 mg, 0.049 mmol) was added and allowed to stir at 50° C. for 16 h. The reaction mixture was concentrated and purified by preparative HPLC to afford Example 660 (3.4 mg, 10% yield). LC-MS Method E: RT=1.703 min, [M+H]⁺=661.3; LC-MS Method F: 1.195 min, [M+H]⁺=661.3; ¹H NMR (400 MHz, DMSO-d₆) δ ppm 9.38 (br s, 1H), 8.00 (br d, J=6.6 Hz, 1H), 7.92 (br d, J=2.2 Hz, 1H), 7.88 (s, 1H), 7.56-7.46 (m, 2H), 6.74 (s, 2H), 5.36 (s, 1H), 4.65 (br s, 1H), 4.58-4.47 (m, 2H), 4.36-4.26 (m, 2H), 4.21 (br dd, J=3.1, 6.0 Hz, 1H), 3.75-3.63 (m, 5H), 3.58-3.48 (m, 1H), 3.28-3.23 (m, 1H), 3.02 (br s, 5H), 2.83-2.70 (m, 2H), 2.30-2.16 (m, 3H), 2.13-1.94 (m, 3H), 1.90-1.74 (m, 4H), 1.61 (br s, 4H).

### Example 661

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-3-(2-(dimethylamino)ethoxy)benzo[b]thiophene-2-carboxamide

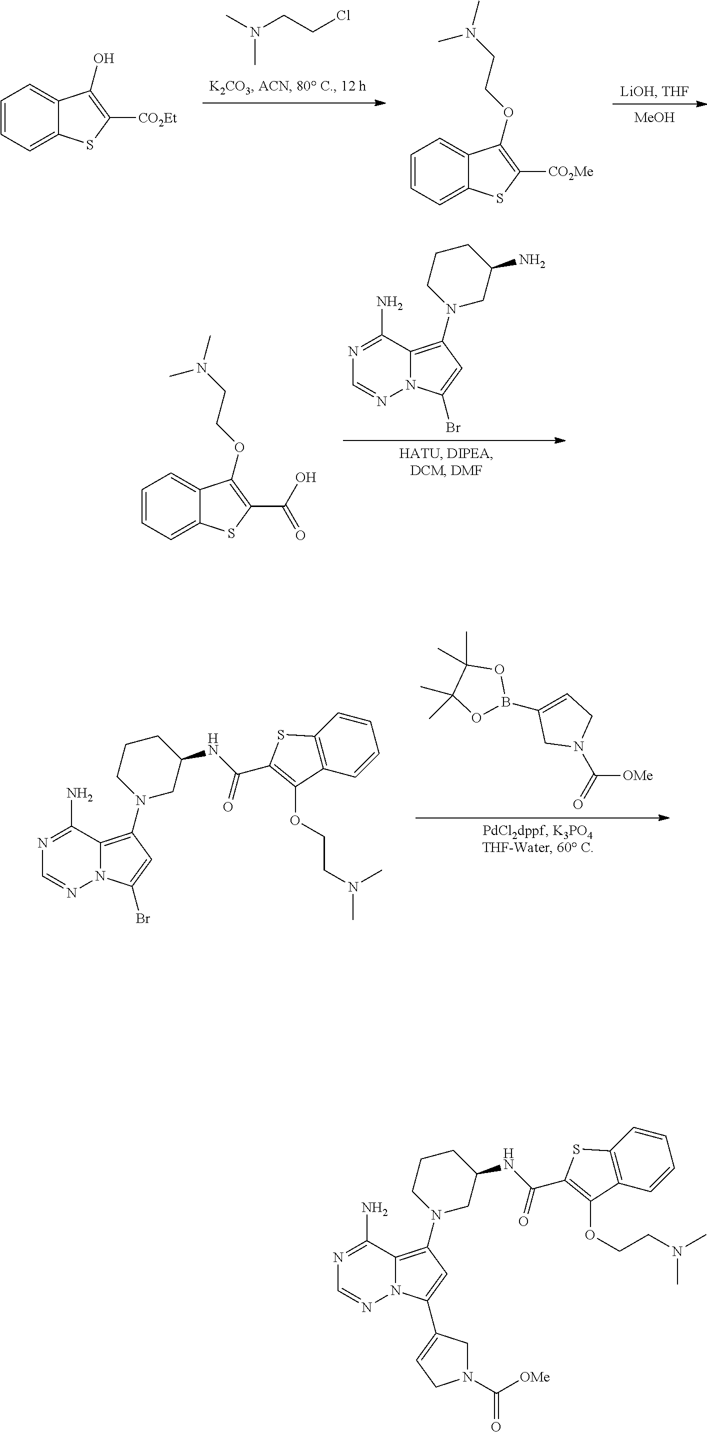

### Example 661A. Methyl 3-(2-(dimethylamino)ethoxy)benzo[b]thiophene-2-carboxylate

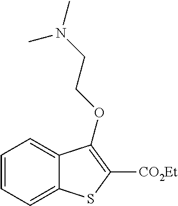 [SMILES: CCOC(=O)c1sc2ccccc2c1OCCN(C)C]

Analogous to the procedure for the preparation of Example 651A, reaction of ethyl 3-hydroxybenzo[b]thiophene-2-carboxylate and 2-chloro-N,N-dimethylethan-1-amine. HCl salt (194 mg, 1.350 mmol) afforded Example 661A (150 mg, 38% yield). MS. [M+H]⁺=294.1.

### Example 661B. 3-(2-(dimethylamino)ethoxy)benzo[b]thiophene-2-carboxylic acid

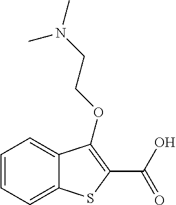 [SMILES: CN(C)CCOc1c(C(=O)O)sc2ccccc12]

Analogous to the procedure for the preparation of Example 376B, reaction of Intermediate 386A with LiOH afforded Example 386B. MS. [M+H]⁺=266.3.

### Example 661C. (R)—N-(1-(4-amino-7-bromopyrrolo[2,1-f][1,2,4]triazin-5-yl)piperidin-3-yl)-3-(2 (dimethylamino)ethoxy)benzo[b]thiophene-2-carboxamide

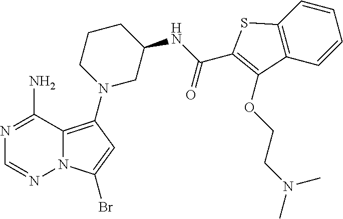 [SMILES: CN(C)CCOc1c(C(=O)N[C@@H]2CCCN(c3cc(Br)n4ncnc(N)c34)C2)sc2ccccc12]

Analogous to the procedure for the preparation of Example 276C, reaction of Intermediate 661B and (R)-5-(3-aminopiperidin-1-yl)-7-bromopyrrolo[2,1-f][1,2,4]triazin-4-amine afforded Example 661C.

The following examples in Table 51 were prepared using the same procedure as shown in Example 276 by coupling Example 661C with respective boronic acids.

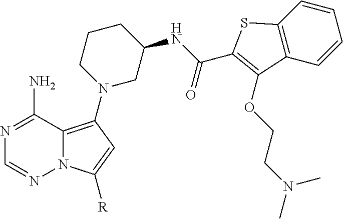

TABLE 51

| | | | | HPLC | |
|---|---|---|---|---|---|
| | | | | Method, | |
| | | | LCMS | RT | |
| Example | R | Name | (M + H)⁺ | (min.) | ¹H NMR |
| 661 | 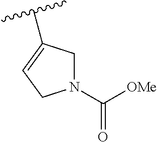 [SMILES: *C1=CCN(C(=O)OC)C1] | Methyl (R)-3-(4-amino-5-(3-(3-(2- (dimethylamino)ethoxy)benzo[b] thiophene-2- carboxamido)piperidin-1- yl)pyrrolo[2,1-f][1,2,4]triazin-7- yl)-2,5-dihydro-1H-pyrrole-1- carboxylate. | 605.3 | A. 1.879 B. 1.137 | (400 MHz, DMSO-d₆) δ ppm 8.27 (br s, 1H), 7.98 (br d, J = 8.0 Hz, 2H), 7.86 (s, 1H), 7.55- 7.45 (m, 2H), 6.77-6.70 (m, 2H), 4.59-4.43 (m, 4H), 4.35-4.25 (m, 6H), 4.24-4.12 (m, 2H), 3.65 (d, J = 4.3 Hz, 3H), 3.26-3.20 (m, 2H), 3.04-2.96 (m, 1H), 2.90-2.68 (m, 4H), 2.01- 1.71 (m, 5H), 1.61-1.47 (m, 1H) |
| 662 | 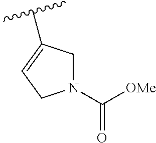 [SMILES: *C1=CCN(C(=O)OC)C1] | (R)-N-(1-(7-(1-acetyl-2,5-dihydro- 1H-pyrrol-3-yl)-4- aminopyrrolo[2,1-f][1,2,4]triazin- 5-yl)piperidin-3-yl)-3-(2- (dimethylamino)ethoxy)benzo[b] thiophene-2-carboxamide. | 589.3 | A. 1.590 B. 0.979 | (400 MHz, DMSO-d₆) δ ppm 8.36-8.25 (m, 1H), 8.03-7.94 (m, 2H), 7.88 (s, 1H), 7.55-7.43 (m, 2H), 6.77 (s, 1H), 6.73 (d, J = 14.2 Hz, 1H), 4.70 (br s, 1H), 4.56-4.44 (m, 7H), 4.30- 4.17 (m, 6H), 3.26 (brd, J = 3.2 Hz, 1H), 3.06- 2.92 (m, 2H), 2.86-2.69 (m, 4H), 2.03 (d, J = 16.1 Hz, 4H), 1.90-1.77 (m, 2H), 1.63-1.46 (m, 2H) |
| 663 | 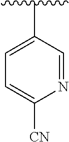 [SMILES: *c1ccc(C#N)nc1] | (R)-N-(1-(4-amino-7-(6- cyanopyridin-3-yl)pyrrolo[2,1- f][1,2,4]triazin-5-yl)piperidin-3- yl)-3-(2- (dimethylamino)ethoxy)benzo[b] thiophene-2-carboxamide. | 582.3 | A. 1.945 B. 1.175 | |
| 664 | 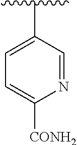 [SMILES: *c1ccc(C(N)=O)nc1] | (R)-5-(4-amino-5-(3-(3-(2- (dimethylamino)ethoxy)benzo[b] thiophene-2- carboxamido)piperidin-1- yl)pyrrolo[2,1-f][1,2,4]triazin-7- yl)picolinamide. | 600.3 | A. 1.595 B. 0.972 | (400 MHz, DMSO-d₆) δ ppm 9.32 (d, J = 1.7 Hz, 1H), 8.69 (dd, J = 2.2, 8.3 Hz, 1H), 8.41- 8.29 (m, 1H), 8.16-8.04 (m, 3H), 8.04-7.96 (m, 3H), 7.93 (s, 1H), 7.64 (br s, 1H), 7.55- 7.43 (m, 3H), 7.31 (s, 1H), 6.98 (s, 1H), 4.58- 4.47 (m, 3H), 4.29-4.18 (m, 1H), 3.11-3.05 (m, 1H), 2.93-2.71 (m, 4H), 2.46-2.35 (m, 3H), 2.04-1.82 (m, 4H), 1.66-1.50 (m, 1H). |

### Example 666

### Preparation of (R)—N-(1-(4-amino-7-(6-cyanopyridin-3-yl)pyrrolo[2,1-f][1,2,4]triazin-5-yl)piperidin-3-yl)-3-(2-(dimethylamino)ethoxy)thieno[3,2-c]pyridine-2-carboxamide

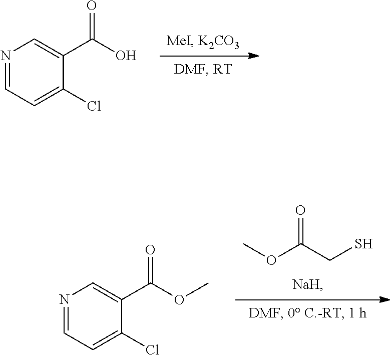

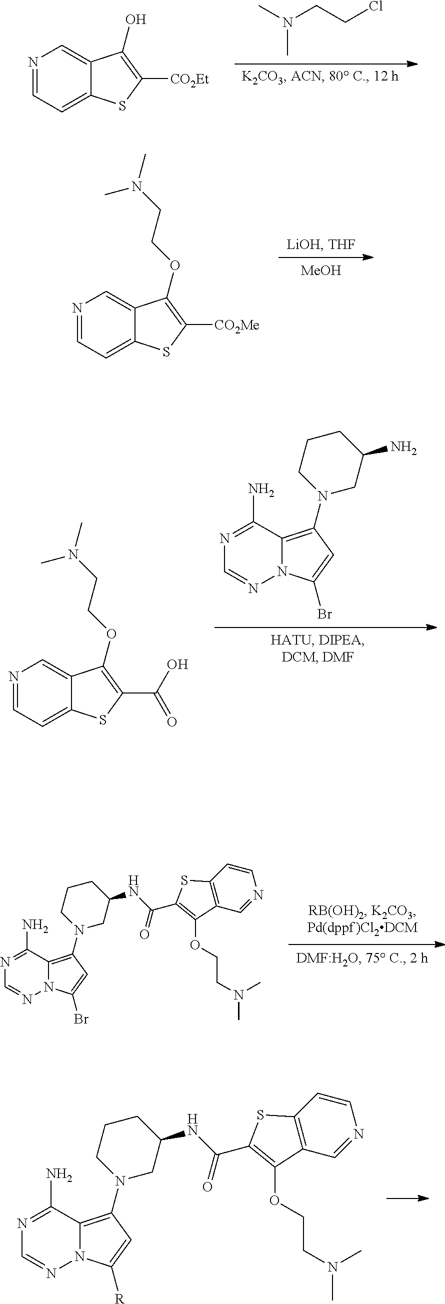

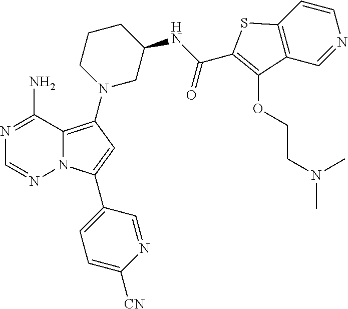 [SMILES: CN(C)CCOc1c(C(=O)N[C@@H]2CCCN(c3cc(-c4ccc(C#N)nc4)n4ncnc(N)c34)C2)sc2ccncc12]

### Example 666A. Preparation of methyl 4-chloronicotinate

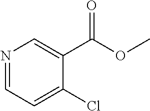

To a solution of 1, 4-chloronicotinic acid (4.0 g, 25.4 mmol) in DMF (40 mL), were added K₂CO₃ (7.02 g, 50.8 mmol) and methyl iodide (3.49 mL, 55.9 mmol). The reaction mixture was stirred for 16 h at rt and filtered through Celite. The filtrate was evaporated under vacuo and the residue was purified by flash chromatography to afford Example 666A (2.6 g, 57% yield). MS: [M+H]⁺=172.1.

### Example 666B. Preparation of methyl 3-hydroxythieno[3,2-c]pyridine-2-carboxylate

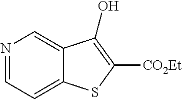 [SMILES: CCOC(=O)c1sc2ccncc2c1O]

To a stirred solution of Example 666A (2.6 g, 13.4 mmol) in DMF (5 mL) at 0° C., methyl thioglycolate (2.86 mL, 30.2 mmol) was added followed by the addition of NaH (1.34 g, 33.5 mmol) in portionwise, maintaining the temperature at 0° C. The temperature of the reaction was raised to RT and stirred for 1 h. The reaction was quenched by the addition of water and the resulting solid was filtered and dried to afford Example 666B (1.5 g, 80% yield). MS: [M−H]⁺=210.0.

### Example 666C. Methyl 3-(2-(dimethylamino)ethoxy)thieno[3,2-c]pyridine-2-carboxylate

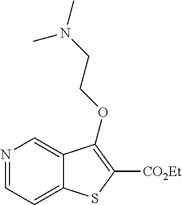 [SMILES: CCOC(=O)c1sc2ccncc2c1OCCN(C)C]

To a solution of Example 666B (1.5 g, 4.78 mmol) in DMF (15 mL), 2-chloro-N, N-dimethylethan-1-amine (1.03 g, 9.56 mmol) and K₂CO₃ (1.98 g, 14.3 mmol) were added and stirred for 16 h at 85° C. Volatiles were evaporated under vacuo and the residue was suspended in cold water and stirred gently for 5 min. Precipitated solid was filtered, washed with water and dried under vacuum to afford Example 666C (0.9 g, 74% yield). MS: [M+H]⁺=281.0.

### Example 666D. Preparation 3-(2-(dimethylamino)ethoxy)thieno[3,2-c]pyridine-2-carboxylic acid

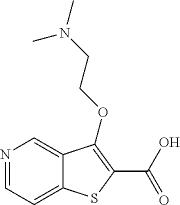 [SMILES: CN(C)CCOc1c(C(=O)O)sc2ccncc12]

To a stirred solution of Example 666C (0.9 g, 3.21 mmol) in THF (5 mL) and water (10.3 mL), was added LiOH monohydrate (0.231 g, 9.63 mmol) at rt. The reaction mass was stirred for 1 h at rt and evaporated under high vacuum to afford Example 666D (0.9 g, 94% yield). MS: [M+H]⁺=267.1.

### Example 666E. Preparation (R)—N-(1-(4-amino-7-bromopyrrolo[2,1-f][1,2,4]triazin-5-yl)piperidin-3-yl)-3-(2-(dimethylamino)ethoxy)thieno[3,2-c]pyridine-2-carboxamide

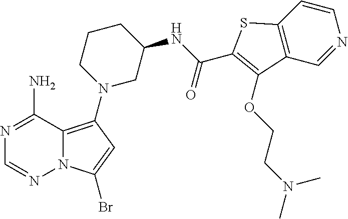 [SMILES: CN(C)CCOc1c(C(=O)N[C@@H]2CCCN(c3cc(Br)n4ncnc(N)c34)C2)sc2ccncc12]

Analogous to the procedure for the preparation of Example 276C, reaction of Example 666D (0.5 g, 3.21 mmol) and (R)-5-(3-aminopiperidin-1-yl)-7-bromopyrrolo[2,1-f][1,2,4]triazin-4-amine afforded Example 666E (0.360 g, 65% yield). MS: [M+H]⁺=560.2.

### Example 666

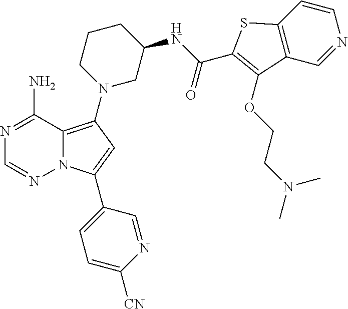 [SMILES: CN(C)CCOc1c(C(=O)N[C@@H]2CCCN(c3cc(-c4ccc(C#N)nc4)n4ncnc(N)c34)C2)sc2ccncc12]

According to the procedure for the preparation of Example 276, coupling of Example 666E (0.150 g) with (6-cyanopyridin-3-yl)boronic acid afforded Example 666 (19 mg, 12% yield). LC-MS Method E: RT=1.585 min, [M+H]⁺=583.2; LC-MS Method F: 0.841 min, [M+H]⁺=583.2; (400 MHz, DMSO-d₆) δ ppm 9.48-9.40 (m, 1H), 9.34 (s, 1H), 8.82 (dd, J=1.8, 8.9 Hz, 1H), 8.53 (d, J=5.6 Hz, 1H), 8.47 (br d, J=7.1 Hz, 2H), 8.30-8.14 (m, 1H), 8.10 (d, J=8.6 Hz, 2H), 8.05 (d, J=5.6 Hz, 1H), 7.97 (s, 1H), 7.43 (s, 1H), 7.02 (s, 1H), 4.28-4.18 (m, 2H), 3.91 (d, J=0.7 Hz, 1H), 3.12-3.04 (m, 1H), 2.90-2.67 (m, 4H), 2.42-2.19 (m, 5H), 2.03-1.82 (m, 3H), 1.57 (br s, 1H)

The following examples in Table 52 were prepared using the same procedure as shown in Example 276. Example 666E was coupled with the appropriate boronic acid or boronate ester.

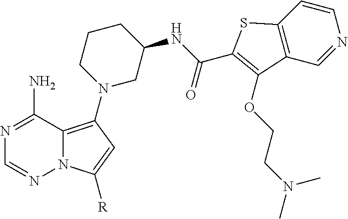

TABLE 52

| | | | | HPLC | |
|---|---|---|---|---|---|
| | | | LCMS | Method, | |
| Example | R | Name | (M + H)⁺ | RT (min.) | ¹H NMR |
| 668 | 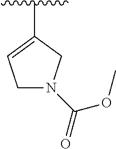 [SMILES: *C1=CCN(C(=O)OC)C1] | methyl (R)-3-(4- amino-5-(3-(3-(2- (dimethylamino) ethoxy)thieno [3,2-c]pyridine-2- carboxamido)piperidin- 1-yl)pyrrolo[2,1-f] [1,2,4]triazin-7- yl)-2,5-dihydro-1H- pyrrole-1-carboxylate | 506.2 | E: 1.539 F: 0.829 | (400 MHz, DMSO-d₆) δ ppm 9.77 (br s, 1H), 9.31 (s, 1H), 8.59 (d, J = 5.9 Hz, 1H), 8.38-8.28 (m, 1H), 8.13 (d, J = 5.9 Hz, 1H), 7.94 (s, 1H), 7.25-6.91 (m, 2H), 6.86-6.71 (m, 2H), 4.66 (t, J = 5.0 Hz, 3H), 4,59- 4.51 (m, 2H), 4.35-4.14 (m,3H), 3.66 (d, J = 4.2 Hz, 3H), 3.63-3.60(m, 3H), 3.28-3.25 (m, 1H), 3.00 (br s, 1H), 2.94 (s, 6H), 2.82-2.71 (m, 3H), 1.63-1.53 (m, 1H) |
| 669 | 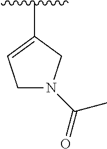 [SMILES: *C1=CCN(C(C)=O)C1] | (R)-N-(1-(7-(1-acetyl- 2,5-dihydro- 1H-pyrrol-3-yl)-4- aminopyrrolo[2,1- f][1,2,4]triazin- 5-yl)piperidin-3-yl)-3-(2- (dimethylamino) ethoxy)thieno [3,2-c]pyridine-2- carboxamide | 590.3 | E: 1.278 F: 0.693 | (400 MHz, DMSO-d₆) δ ppm 9.34 (s, 1H), 8.52 (d, J = 5.6 Hz, 1H), 8.44 (d, J = 7.8 Hz, 1H), 8.04 (d, J = 5.6 Hz, 1H), 7.88 (s, 1H), 7.25- 6.91 (m, 1H), 6.81-6.68 (m, 2H), 4.72- 4.59 (m, 3H), 4.53-4.42 (m, 3H), 4.26 (br s, 2H), 4.22 (br d, J = 5.1 Hz, 2H), 3.24 (m, 1H), 3.02 (br s, 1H), 2.81-2.63 (m,4H), 2.23 (s, 5H), 2.03 (d, J = 16.4 Hz, 3H), 1.87 (m, 2H), 1.52 (br s, 1H) |
| 670 | 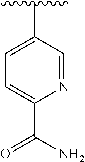 [SMILES: *c1ccc(C(N)=O)nc1] | (R)-N-(1-(4-amino-7-(6- carbamoylpyridin-3- yl)pyrrolo[2,1-f] [1,2,4]triazin-5- yl)piperidin-3-yl)-3-(2- (dimethylamino) ethoxy)thieno [3,2-c]pyridine- 2-carboxamide | 601.7 | E: 1.293 F: 0.678 | (400 MHz, DMSO-d₆) δ ppm 9.34 (s, 1H), 9.32 (d, J = 2.0 Hz, 1H), 8.69 (dd, J = 2.2, 8.3 Hz, 1H), 8.52 (d, J = 5.6 Hz, 1H), 8.47 (d, J = 7.8Hz, 1H), 8.21-8.00 (m, 4H), 7.93 (s, 1H), 7.64 (br s, 1H), 7.32 (s, 1H), 6.98 (br s, 1H), 4.73-4.55 (m, 2H), 4.29-4.17 (m, 1H), 3.30 (br d, J = 1.5Hz, 1H), 3.13-3.04 (m, 1H), 2.92- 2.60 (m, 4H), 2.27 (br s, 6H), 2.04-1.75 (m, 3H), 1.67-1.47 (m, 1H). |

### Example 671

### Preparation of methyl 3-(4-amino-5-((R)-3-(3-((1-(tert-butoxycarbonyl)azetidin-3-yl)oxy)benzo[b]thiophene-2-carboxamido)piperidin-1-yl)pyrrolo[2,1-f][1,2,4]triazin-7-yl)pyrrolidine-1-carboxylate

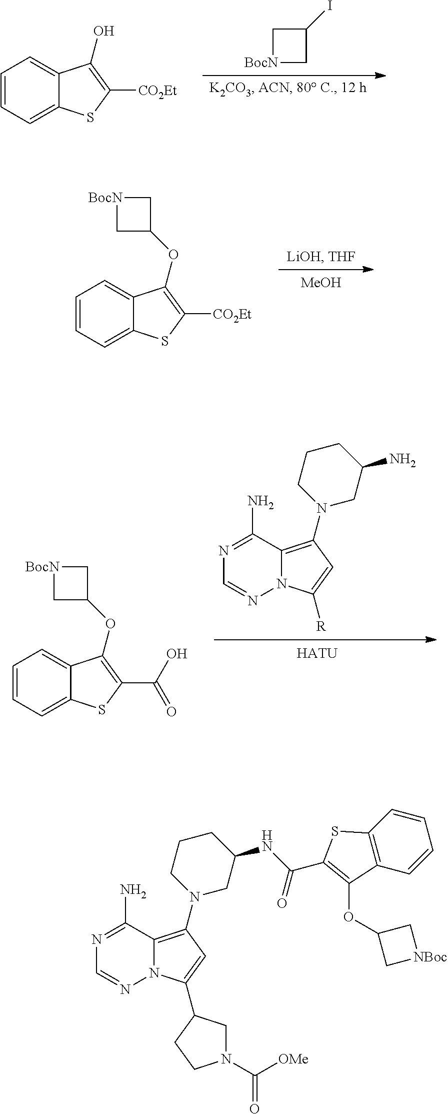

### Example 671A. Preparation of tert-butyl 3-((2-(ethoxycarbonyl)benzo[b]thiophen-3-yl)oxy)azetidine-1-carboxylate

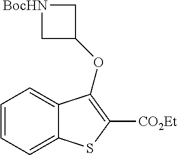

Analogous to the procedure for the preparation of Example 671A, reaction of ethyl 3-hydroxybenzo[b]thiophene-2-carboxylate (300 mg, 1.35 mmol) and tert-butyl 3-iodoazetidine-1-carboxylate (0.234 mL, 1.35 mmol) afforded Example 671A (0.9 g, 74% yield). MS: [M−56+H]⁺=322.2.

### Example 671B. Preparation of 3-((1-(tert-butoxycarbonyl)azetidin-3-yl)oxy)benzo[b]thiophene-2-carboxylic acid

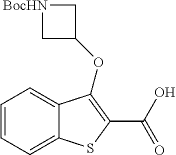

Analogous to the procedure for the preparation of Example 651B, reaction of Example 671A with LiOH afforded Example 671B. MS: [M−56+H]⁺=294.2

### Example 671

Reaction of Intermediate 31 and Example 671B in presence of HATU and DIEA afforded Example 671. LC-MS Method E: RT=2.063 min, [M+H]⁺=689.3; LC-MS Method F: RT=1.752 min, [M+H]⁺=689.3; ¹H NMR (400 MHz, DMSO-d₆) δ ppm 8.15-8.02 (m, 1H), 8.02-7.92 (m, 1H), 7.88 (s, 1H), 7.85 (dd, J=1.3, 7.0 Hz, 1H), 7.60-7.37 (m, 2H), 6.82-6.64 (m, 2H), 5.34-5.23 (m, 1H), 4.60-4.48 (m, 2H), 4.35-4.20 (m, 3H), 4.17 (br dd, J=6.2, 9.4 Hz, 3H), 4.05-3.95 (m, 2H), 3.65 (d, J=2.7 Hz, 3H), 3.23 (br dd, J=2.3, 9.2 Hz, 1H), 3.01-2.94 (m, 1H), 2.88-2.69 (m, 2H), 1.98-1.74 (m, 3H), 1.67-1.50 (m, 1H), 1.32 (d, J=2.2 Hz, 10H).

The following examples in Table 53 were prepared using the same procedure as shown in Example 671. Example 671B was coupled with the appropriate amines in presence of HATU and DIEA.

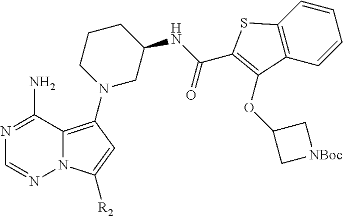

TABLE 53

| | | | | HPLC | |
|---|---|---|---|---|---|
| | | | LCMS | Method, | |
| Example | R₂ | Name | (M + H)⁺ | RT (min.) | ¹H NMR |
| 672 | 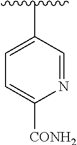 [SMILES: *c1ccc(C(N)=O)nc1] | tert-butyl (R)-3- ((2-((1-(4-amino- 7-(6-cyanopyridin-3- yl)pyrrolo[2,1-f] [1,2,4]triazin-5- yl)piperidin-3- yl)carbamoyl) benzo[b]thiophen- 3-yl)oxy)azetidine- 1-carboxylate | 684.3 | E: 1.818 F: 1.587 | (400 MHz, DMSO-d₆) δ ppm 9.32 (d, J = 2.0 Hz, 1H), 8.70 (dd, J = 2.2, 8.3 Hz, 1H), 8.17- 8.06 (m, 3H), 8.01 (s, 1H), 7.93 (s, 1H), 7.89- 7.85 (m, 1H), 7.64 (br d, J = 2.2 Hz, 1H), 7.55-7.47 (m, 2H), 7.32 (s, 1H), 5.34-5.27 (m, 1H), 4.29 (br d, J = 5.1 Hz, 2H), 4.20-4.15 (m, 3H), 4.04-3.99 (m, 2H), 3.03 (s, 1H), 2.92- 2.75 (m, 2H), 1.89 (br d, J = 2.0 Hz, 3H), 1.70-1.57 (m, 1H), 1.28 (s, 10H). |
| 673 | 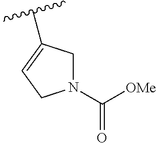 [SMILES: *C1=CCN(C(=O)OC)C1] | tert-butyl (R)- 3-((2-((1-(7-(1- acetyl-2,5-dihydro- 1H-pyrrol-3- yl)-4-aminopyrrolo [2,1-f][1,2,4]triazin- 5-yl)piperidin-3- yl)carbamoyl) benzo[b]thiophen- 3-yl)oxy)azetidine- 1-carboxylate | 673.3 | E: 1.817 F: 1.574 | (400 MHz, DMSO-d₆) δ ppm 8.12-7.96 (m, 1H), 7.88-7.80 (m, 1H), 7.58-7.45 (m, 2H), 7.25 (s, 2H), 7.12 (s, 2H), 7.00 (s, 2H), 6.83-6.72 (m, 1H), 4.70 (br d, J = 1.7 Hz, 1H), 5.30-5.20 (m, 1H) 4.54-4.44 (m, 2H), 4.39-4.11 (m, 5H), 4.00 (br s, 2H), 3.24 (br d, J = 9.0 Hz, 1H), 3.06-2.74 (m, 3H), 2.10-2.01 (m, 3H), 1.97- 1.77 (m, 1H), 1.67-1.53 (m, 1H), 1.32 (d, J = 3.7 Hz, 9H) |

### Example 674

### Preparation of methyl (R)-3-(4-amino-5-(3-(3-(azetidin-3-yloxy)benzo[b]thiophene-2-carboxamido)piperidin-1-yl)pyrrolo[2,1-f][1,2,4]triazin-7-yl)-2,5-dihydro-1H-pyrrole-1-carboxylate

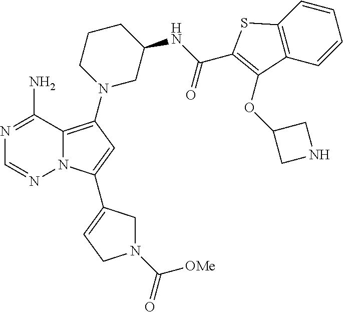 [SMILES: COC(=O)N1CC=C(c2cc(N3CCC[C@@H](NC(=O)c4sc5ccccc5c4OC4CNC4)C3)c3c(N)ncnn23)C1]

Reaction of Example 671 (40 mg, 0.058 mmol) with TFA afforded Example 674. LC-MS Method E: RT=1.433 min, [M+H]⁺=589.2; LC-MS Method F: 1.109 min, [M+H]⁺=589.3.

### Example 675

### Preparation of (R)-5-(4-amino-5-(3-(3-(azetidin-3-yloxy)benzo[b]thiophene-2-carboxamido)piperidin-1-yl)pyrrolo[2,1-f][1,2,4]triazin-7-yl)picolinamide

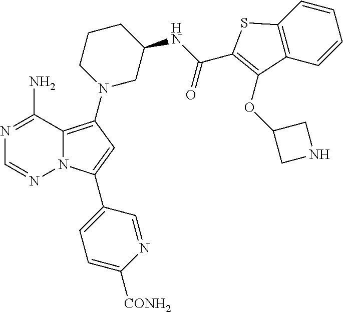 [SMILES: NC(=O)c1ccc(-c2cc(N3CCC[C@@H](NC(=O)c4sc5ccccc5c4OC4CNC4)C3)c3c(N)ncnn23)cn1]

Analogous to the procedure for the preparation of Example 651, reaction of Example 672 with TFA afforded Example 675. LC-MS Method E: RT=1.235 min, [M+H]⁺=589.2; LC-MS Method F. 0.943 min, [M+H]⁺=584.2; ¹H NMR (400 MHz, DMSO-d₆) δ ppm 9.31 (d, J=1.5 Hz, 1H), 8.69 (dd, J=2.3, 8.5 Hz, 1H), 8.26-8.19 (m, 1H), 8.15-8.02 (m, 3H), 8.01-7.95 (m, 2H), 7.95-7.91 (m, 1H), 7.85 (d, J=7.3 Hz, 1H), 7.69-7.60 (m, 2H), 7.58-7.46 (m, 2H), 7.31 (s, 1H), 5.30-5.20 (m, 1H), 4.33-4.17 (m, 2H), 4.16-4.07 (m, 2H), 4.06-3.95 (m, 1H), 3.06-3.00 (m, 1H), 2.95-2.74 (m, 3H), 2.02-1.76 (m, 3H), 1.70-1.59 (m, 1H).

### Example 676

### Preparation of tert-butyl (R)-4-((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-cyanobenzo[b]thiophen-3-yl)oxy)piperidine-1-carboxylate

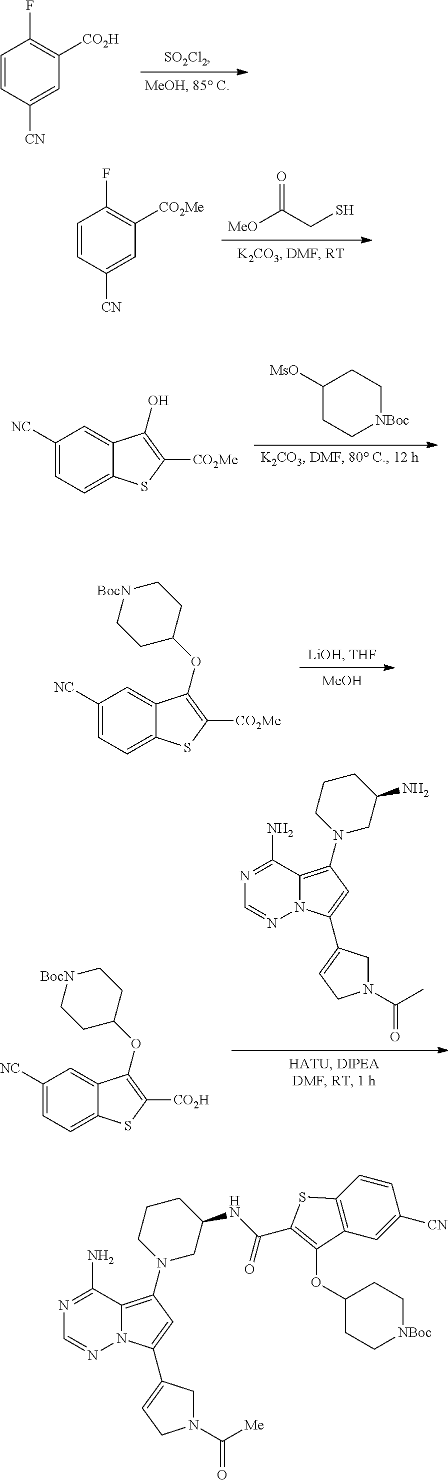

### Example 676A. Preparation of methyl 5-cyano-2-fluorobenzoate

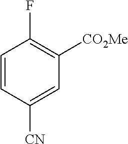 [SMILES: COC(=O)c1cc(C#N)ccc1F]

To a solution of 5-cyano-2-fluorobenzoic acid (5 g, 30.3 mmol) in MeOH (20 mL), was added thionyl chloride (1.11 mL, 15.1 mmol) at rt. The mixture was stirred for 4 h at 75° C. and evaporated under vacuo to afford Example 676A (5.0 g, 92% yield) as a white solid. ¹H NMR (400 MHz, DMSO-d₆) δ ppm 8.35 (dd, J=2.0, 7.0 Hz, 1H), 8.20 (ddd, J=2.3, 4.5, 8.8 Hz, 1H), 7.63 (dd, J=8.5, 10.5 Hz, 1H), 3.91-3.88 (m, 3H). ¹⁹F NMR δ ppm −101.24 ppm.

### Example 676B. Preparation of methyl 5-cyano-3-hydroxybenzo[b]thiophene-2-carboxylate

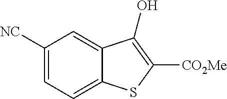 [SMILES: COC(=O)c1sc2ccc(C#N)cc2c1O]

To a stirred solution of Example 676A in DMF (5 mL), K₂CO₃ (9.64 g, 69.8 mmol) and methyl thioglycolate. (5.95 mL, 62.8 mmol) were added at it and stirred for 2 h. Water was added solids formed was filtered and dried to afford Example 676B (5 g, 77% yield). MS: [M−H]⁺=232.0; ¹H NMR (400 MHz, DMSO-d₆) δ ppm 7.96 (s, 1H), 7.71 (d, J=8.0 Hz, 1H), 7.60-7.56 (m, 1H), 3.58 (s, 3H).

### Example 401C. Preparation of tert-butyl 4-((5-cyano-2-(methoxycarbonyl)benzo[b]thiophen-3-yl)oxy)piperidine-1-carboxylate

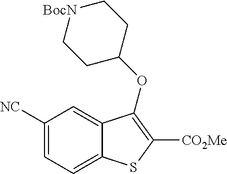 [SMILES: COC(=O)c1sc2ccc(C#N)cc2c1OC1CCN(C(=O)OC(C)(C)C)CC1]

Analogous to the procedure for the preparation of Example 651, reaction of Example 676B (651 mg, 2.79 mmol) and tert-butyl 4-((methylsulfonyl)oxy)piperidine-1-carboxylate (1170 mg, 4.19 mmol) afforded Example 676C (800 mg, 69% yield). MS: [M−56+H]⁺=362.3; ¹H NMR (400 MHz, DMSO-d₆) δ ppm 8.49 (dd, J=1.0, 7.5 Hz, 1H), 8.24 (dd, J=2.5, 8.5 Hz, 1H), 7.93 (d, J=8.6 Hz, 1H), 4.04 (q, J=7.2 Hz, 1H), 3.84 (s, 3H), 3.90-3.81 (m, 2H), 3.70-3.57 (m, 2H), 3.30-2.55-2.52 (m, 2H), 1.97-1.88 (m, 1H), 1.77-1.54 (m, 1H), 1.44-1.39 (m, 9H).

### Example 676D. Preparation of 3-((1-(tert-butoxycarbonyl)piperidin-4-yl)oxy)-5-cyanobenzo[b]thiophene-2-carboxylic acid

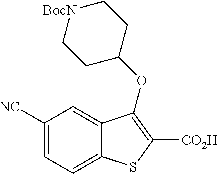 [SMILES: CC(C)(C)OC(=O)N1CCC(Oc2c(C(=O)O)sc3ccc(C#N)cc23)CC1]

Example 676C (100 mg, 0.24 mmol) was added to a solution of LiOH (5.8 mg, 0.24 mmol) in THF (3 mL), MeOH (5 mL) and water (3 mL). The mixture was stirred at rt for 2 h. The reaction mixture was evaporated to dryness, suspended in water (10 mL). and acidified at 10° C. with 1N HCl to pH 2. Extracted with ethyl acetate (3×) and the organic layer was dried over sodium sulphate, filtered and evaporated under vacuo to afford Example 676D (80 mg, 83% yield). MS: [M−H]⁺=401.2; ¹H NMR (400 MHz, DMSO-d₆) δ ppm 13.4 (bs, 1H), 7.89 (dd, J=1.5, 8.5 Hz, 1H), 4.12-3.98 (m, 1H), 3.88-3.75 (m, 1H), 3.69-3.54 (m, 1H), 3.52-3.37 (m, 1H), 3.29-3.18 (m, 2H), 3.03 (br s, 1H), 2.63-2.53 (m, 1H), 1.95-1.87 (m, 1H), 1.76-1.54 (m, 2H), 1.44-1.37 (m, 9H).

### Example 676

Analogous to the procedure for the preparation of Example 561C, Reaction of Example 676D and Intermediate 32 afforded Example 676. LC-MS Method E: RT=1.853 min, [M+H]⁺=726.3; LC-MS Method F: 1.610 min, [M+H]⁺=726.4; ¹H NMR (400 MHz, DMSO-d₆) δ ppm 8.51 (s, 1H), 8.23 (d, J=8.3 Hz, 1H), 8.09 (dt, J=1.3, 2.9 Hz, 1H), 7.97-7.91 (m, 1H), 7.86 (d, J=8.3 Hz, 1H), 7.29-6.91 (m, 2H), 6.84-6.62 (m, 2H), 4.70 (br s, 2H), 4.56-4.44 (m, 2H), 4.33-4.17 (m, 2H), 3.82 (dt, J=3.2, 6.0 Hz, 2H), 3.23 (br dd, J=2.0, 10.5 Hz, 1H), 3.01-2.71 (m, 5H), 2.03 (d, J=15.4 Hz, 4H), 1.97-1.78 (m, 4H), 1.65-1.50 (m, 3H), 1.35 (d, J=2.4 Hz, 9H).

### Example 677

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-cyano-3-(piperidin-4-yloxy)benzo[b]thiophene-2-carboxamide

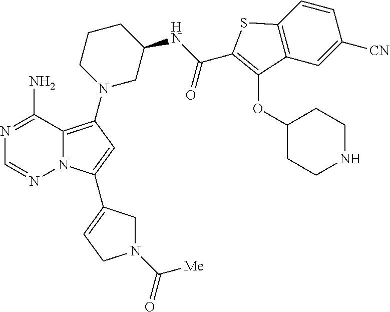 [SMILES: CC(=O)N1CC=C(c2cc(N3CCC[C@@H](NC(=O)c4sc5ccc(C#N)cc5c4OC4CCNCC4)C3)c3c(N)ncnn23)C1]

Reaction of Example 676 with TFA afforded Example 677. LC-MS Method A. RT=1.261 min, [M+H]⁺=626.2; LC-MS Method F. 1.023 min, [M+H]⁺=626.3; ¹H NMR (400 MHz, DMSO-d) δ ppm 8.95 (s, 1H), 8.76-8.71 (m, 2H), 8.61-8.56 (m, 1H), 8.40-8.32 (m, 5H), 7.30-7.19 (m, 3H), 5.25-5.08 (m, 1H), 5.02-4.90 (m, 1H), 4.79-4.71 (m, 2H), 3.55 (td, J=1.3, 11.0 Hz, 5H), 3.34-3.27 (m, 4H), 2.58-2.37 (m, 5H), 2.17 (m, 4H), 1.35 (s, 1H).

### Example 678

### Preparation of tert-butyl (R)-4-((2-((1-(4-amino-7-(1-(methoxycarbonyl)-2,5-dihydro-1H-pyrrol-3-yl) pyrrolo[2,1-f][1,2,4]triazin-5-yl)piperidin-3-yl)carbamoyl)-5-cyanobenzo[b]thiophen-3-yl)oxy)piperidine-1-carboxylate

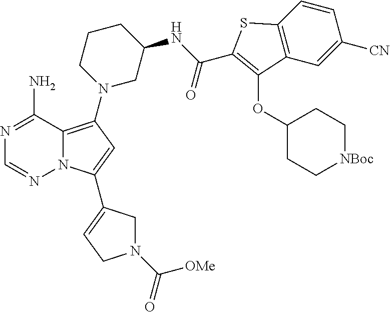 [SMILES: COC(=O)N1CC=C(c2cc(N3CCC[C@@H](NC(=O)c4sc5ccc(C#N)cc5c4OC4CCN(C(=O)OC(C)(C)C)CC4)C3)c3c(N)ncnn23)C1]

Analogous to the procedure for the preparation of Example 561C, reaction of Example 676D and Intermediate 31 afforded Example 678. LC-MS Method E: RT=2.093 min, [M+H]⁺=742.3; LC-MS Method F. 1.779 min, [M+H]⁺=742.4; ¹H NMR (400 MHz, DMSO-d₆) δ ppm 8.51 (s, 1H), 8.23 (d, J=8.6 Hz, 1H), 8.09 (br s, 2H), 7.90-7.79 (m, 2H), 6.74 (br s, 2H), 4.78-4.66 (m, 1H), 4.59-4.45 (m, 2H), 4.34-4.17 (m, 3H), 3.87-3.73 (m, 3H), 3.66 (d, J=2.7 Hz, 3H), 3.23-3.16 (m, 1H), 3.05-2.70 (m, 5H), 2.07-1.73 (m, 5H), 1.65-1.46 (m, 3H), 1.35 (s, 9H).

### Example 679

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-7-cyanoimidazo[1,2-a]pyridine-2-carboxamide

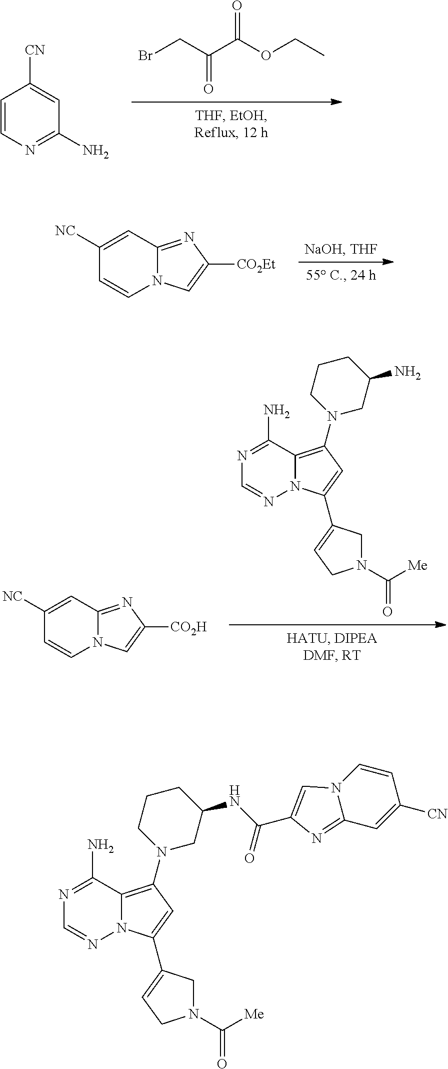

### Example 679A. Preparation of ethyl 7-cyanoimidazo[1,2-a]pyridine-2-carboxylate

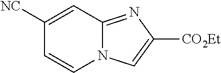 [SMILES: CCOC(=O)c1cn2ccc(C#N)cc2n1]

To a solution of 2-aminoisonicotinonitrile (1 g, 8.39 mmol) in EtOH (10 mL), ethyl bromopyruvate (1.06 mL, 8.39 mmol) was added and reaction mixture was stirred at reflux conditions. The mixture was concentrated under high vacuo to afford Example 679A (1.8 g, 100% yield). MS: [M−H]⁺=216.2.

### Example 679B. Preparation of 7-cyanoimidazo[1,2-a]pyridine-2-carboxylic acid

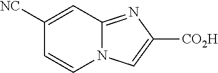 [SMILES: N#Cc1ccn2cc(C(=O)O)nc2c1]

To a suspension of Example 679A (1.5 g, 6.97 mmol) in a mixture of dioxane (15 mL) and water (5 mL), sodium hydroxide (0.362 g, 9.06 mmol) was added and stirred at 50° C. overnight. Reaction was concentrated, acidified with 4N HCl in dioxane and concentrated to afford Example 679 (1.0 g, 77% yield). MS. [M−H]⁺=188.1.

### Example 679

Analogous to the procedure for the preparation of Example 561C, reaction of Intermediate 32 and Example 679B afforded Example 679. LC-MS Method E: RT=1.208 min, [M+H]⁺=511.3; LC-MS Method F: 0.968 min, [M+H]⁺=511.3; ¹H NMR (400 MHz, DMSO-d₆) δ ppm 8.79-8.70 (m, 1H), 8.57 (s, 1H), 8.46 (dd, J=5.4, 8.1 Hz, 1H), 8.41 (s, 1H), 7.94 (s, 1H), 7.31 (dd, J=1.2, 7.1 Hz, 1H), 7.25-6.94 (m, 1H), 6.83-6.74 (m, 2H), 4.70 (br d, J=2.4 Hz, 2H), 4.51 (br d, J=2.7 Hz, 1H), 4.48 (br s, 1H), 4.31-4.19 (m, 2H), 3.19-3.15 (m, 1H), 3.01 (br d, J=14.4 Hz, 1H), 2.85-2.69 (m, 2H), 2.03 (d, J=15.7 Hz, 3H), 1.93-1.60 (m, 4H).

### Example 682

### Preparation of (R)—N-(1-(4-amino-7-(1-ethyl-6-oxo-1,6-dihydropyridin-3-yl)pyrrolo[2,1-f][1,2,4]triazin-5-yl)piperidin-3-yl)-5-chloro-3-(2-(dimethylamino)ethoxy)thiophene-2-carboxamide

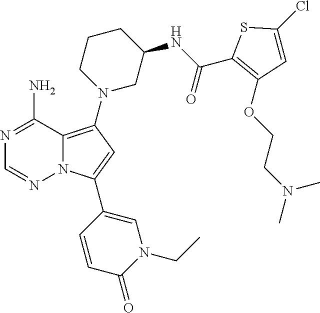 [SMILES: CCn1cc(-c2cc(N3CCC[C@@H](NC(=O)c4sc(Cl)cc4OCCN(C)C)C3)c3c(N)ncnn23)ccc1=O]

Analogous to the procedure for the preparation of Example 276, reaction of Example 276C and Intermediate 10 afforded Example 682. LCMS Method E: RT=1.833 min, [M+H]⁺=587.1; LCMS Method F: RT=1.827 min, [M+H]⁺=587.1; ¹H NMR (400 MHz, DMSO-d₆) δ ppm 7.88-7.81 (m, 2H), 7.61-7.35 (m, 2H), 7.24-6.90 (m, 2H), 6.19 (dd, J=3.0, 5.5 Hz, 1H), 5.29 (s, 1H), 4.49 (br s, 1H), 4.23 (br s, 1H), 4.09 (t, 2H), 4.05-3.96 (m, 4H), 3.13-3.04 (m, 5H), 1.78-1.55 (m, 3H), 1.18 (t, J=7.1 Hz, 6H), 0.96 (d, 3H).

### Example 683

### Preparation of (R)—N-(1-(4-amino-7-(1-(difluoromethyl)-6-oxo-1,6-dihydropyridin-3-yl)pyrrolo[2,1-f][1,2,4]triazin-5-yl)piperidin-3-yl)-5-chlorothiophene-2-carboxamide

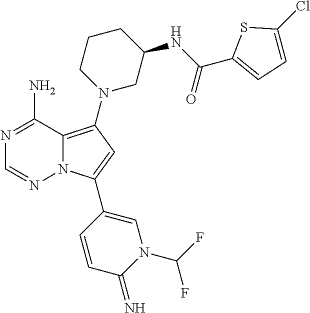 [SMILES: N=c1ccc(-c2cc(N3CCC[C@@H](NC(=O)c4ccc(Cl)s4)C3)c3c(N)ncnn23)cn1C(F)F]

Analogous to the procedure for the preparation of Example 479, reaction of Example 479A and 1-(difluoromethyl)-5-(4,4,5,5-tetramethyl-1,3,2-dioxaborolan-2-yl) pyridin-2(1H)-imine (62.2 mg, 0.230 mmol) afforded Example 683. LCMS Method E: RT=1.474 min, [M+H]⁺=519.1; LCMS Method F: RT=1.474 min, [M+H]⁺=519.1; ¹H NMR (400 MHz, DMSO-d₆) δ ppm 8.92 (br s, 1H), 8.47 (d, J=7.8 Hz, 1H), 8.30-8.23 (m, 1H), 8.11-7.78 (m, 1H), 7.72 (d, J=4.2 Hz, 1H), 7.21-7.16 (m, 1H), 7.14 (s, 1H), 7.03 (s, 1H), 4.17-4.10 (m, 1H), 3.03 (br d, J=1.2 Hz, 1H), 2.74-2.66 (m, 2H), 1.90 (br d, J=14.4 Hz, 3H), 1.57-1.50 (m, 1H).

### Example 684

### Preparation of methyl (R)-3-(4-amino-5-(3-(5-cyano-1H-indazole-3-carboxamido)piperidin-1-yl)pyrrolo[2,1-f][1,2,4]triazin-7-yl)-2,5-dihydro-1H-pyrrole-1-carboxylate

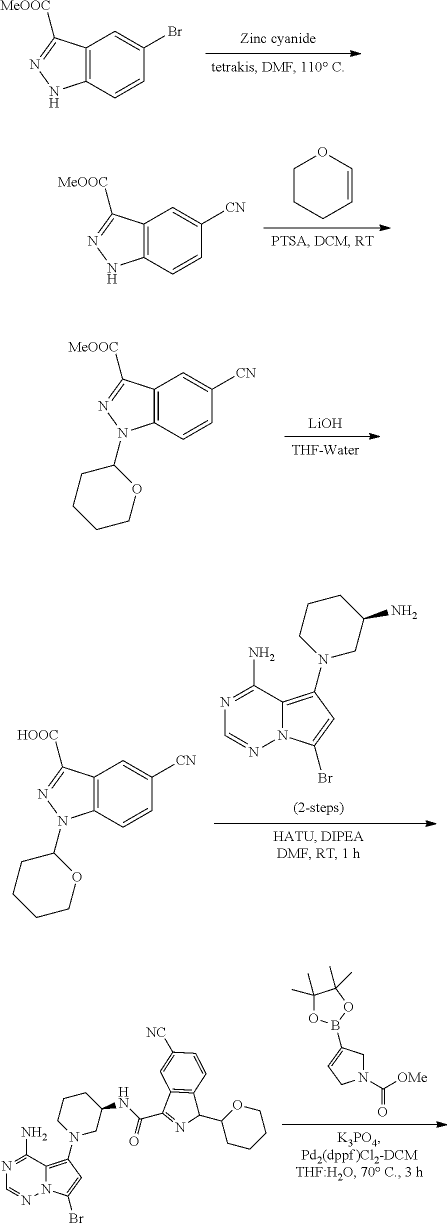

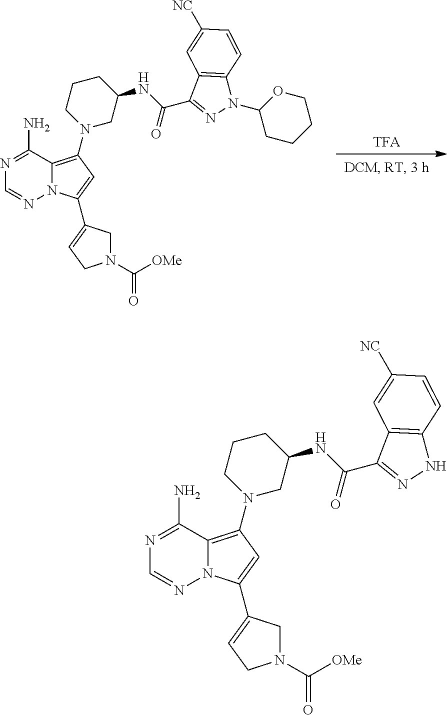 [SMILES: COC(=O)N1CC=C(c2cc(N3CCC[C@@H](NC(=O)c4nn(C5CCCCO5)c5ccc(C#N)cc45)C3)c3c(N)ncnn23)C1 ; "DCM, RT, 3 h" ; COC(=O)N1CC=C(c2cc(N3CCC[C@@H](NC(=O)c4n[nH]c5ccc(C#N)cc45)C3)c3c(N)ncnn23)C1 ; "TFA"]

### Example 684A. Preparation of methyl 6-cyano-1H-indazole-3-carboxylate

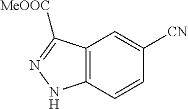 [SMILES: COC(=O)c1n[nH]c2ccc(C#N)cc12]

To a stirred solution of methyl 6-bromo-1H-indazole-3-carboxylate (3.0 g, 11.8 mmol) in DMF (30 mL), was added zinc cyanide (2.76 g, 23.5 mmol). The reaction mixture was purged with N₂ for 15 mini followed by the addition of Pd(PPh₃)₄ (1.359 g, 1.18 mmol) at rt. Then, it was heated at 100° C. for 16 h. After cooling to rt, ethyl acetate (80 mL) and water (100 mL) were added. Organic layer was separated, dried over anhydrous sodium sulphate, filtered and concentrated. The crude product was purified by flash chromatography (ethyl acetate in pet ether) to get Example 684A (2.1 g, 89% yield) as a brown solid. MS: [M−H]⁺=200.1.

### Example 684B. Preparation of methyl 5-cyano-1-(tetrahydro-2H-pyran-2-yl)-1H-indazole-3-carboxylate

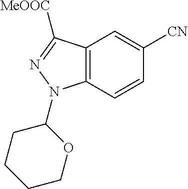 [SMILES: COC(=O)c1nn(C2CCCCO2)c2ccc(C#N)cc12]

To a stirred solution of 3,4-dihydro-2H-pyran (0.627 g, 7.46 mmol) in DCM (20 mL), were added Example 684A (1 g, 4.97 mmol) and PTSA (0.095 g, 0.497 mmol) at rt. The reaction mixture was stirred at 80° C. for 16 h. Then, it was diluted with dichloromethane (50 mL) followed by the addition of aq. 10% NaHCO₃ solution (100 mL). Organic layer was separated, dried over anhydrous sodium sulphate, filtered and concentrated. The crude product was purified by flash chromatography (4% ethyl acetate in pet-ether) to get Example 684B (1.1 g, 78% yield) as an off-white solid. It was a mixture of two regioisomers. MS: [M+H]⁺=286.6.

### Example 684C. Preparation of 5-cyano-1-(tetrahydro-2H-pyran-2-yl)-1H-indazole-3-carboxylic acid, lithium salt

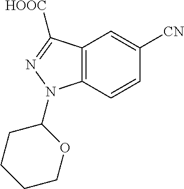 [SMILES: N#Cc1ccc2c(c1)c(C(=O)O)nn2C1CCCCO1]

To a stirred solution of Example 684B (0.1 g, 0.351 mmol) in THF (2 mL) and water (2 mL), was added LiOH (0.017 g, 0.701 mmol) at rt. The reaction mixture was stirred at rt for 12 h and concentrated to get Example 684C as lithium salt (0.095 g, 97% yield) as an off-white solid. MS: [M−H]⁺=270.1.

### Example 684D. Preparation of N—((R)-1-(4-amino-7-bromopyrrolo[2,1-f][1,2,4]triazin-5-yl)piperidin-3-yl)-5-cyano-1-(tetrahydro-2H-pyran-2-yl)-1H-indazole-3-carboxamide

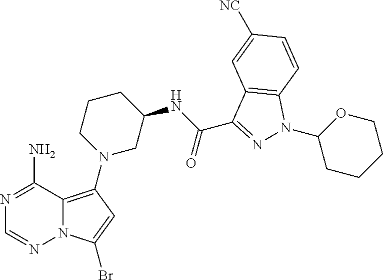 [SMILES: N#Cc1ccc2c(c1)c(C(=O)N[C@@H]1CCCN(c3cc(Br)n4ncnc(N)c34)C1)nn2C1CCCCO1]

Analogous to the procedure for the preparation of Example 276C, reaction of Example 684C and tert-butyl (R)-(1-(4-amino-7-bromopyrrolo[2,1-f][1,2,4]triazin-5-yl)piperidin-3-yl)carbamate (0.574 g, 1.84 mmol) afforded Example 684D (150 mg, 14% yield) as a brown solid. MS: [M+H]⁺=564.2

### Example 684E. Preparation of methyl 3-(4-amino-5-((3R)-3-(5-cyano-1-(tetrahydro-2H-pyran-2-yl)-1H-indazole-3-carboxamido) piperidin-1-yl) pyrrolo[2,1-f][1,2,4] triazin-7-yl)-2,5-dihydro-1H-pyrrole-1-carboxylate

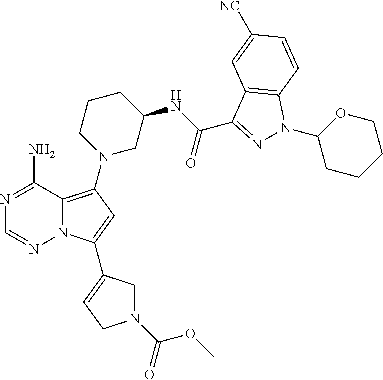 [SMILES: COC(=O)N1CC=C(c2cc(N3CCC[C@@H](NC(=O)c4nn(C5CCCCO5)c5ccc(C#N)cc45)C3)c3c(N)ncnn23)C1]

Analogous to the procedure for the preparation of Example 276, reaction of Example 684D and Intermediate 28 afforded to get Example 684E as a brown gummy solid. MS: [M+H]⁺=611.3.

### Example 684

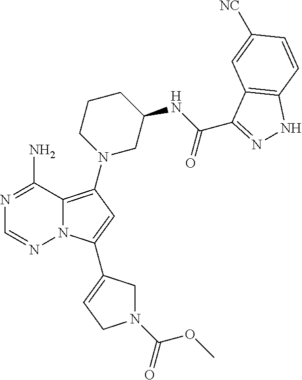 [SMILES: COC(=O)N1CC=C(c2cc(N3CCC[C@@H](NC(=O)c4n[nH]c5ccc(C#N)cc45)C3)c3c(N)ncnn23)C1]

To a stirred solution of Example 684E (0.28 g, 0.138 mmol) in DCM (10 mL), was added TFA (10.60 μl, 0.138 mmol) at rt. The reaction mixture was stirred at rt for 2 b and concentrated. The crude product was purified by preparative HPLC to afford Example 684. LC-MS Method E: RT=1.645 min, [M+H]⁺=527.2; LC-MS Method F: RT-1.32 min, [M+H]⁺=527.3; ¹H NMR (400 MHz, DMSO-d₆) δ ppm 14.19 (br s, 1H), 8.94-8.80 (m, 1H), 8.49 (d, J=8.3 Hz, 1H), 8.45-8.36 (m, 1H), 8.35-8.29 (m, 1H), 8.28 (s, 1H), 7.94 (s, 1H), 7.57 (dd, J=1.2, 8.6 Hz, 1H), 7.27 (br s, 1H), 6.81 (d, J=4.9 Hz, 1H), 6.76 (br s, 1H), 4.60-4.49 (m, 2H), 4.37-4.23 (m, 3H), 3.65 (br s, 3H), 3.24-3.19 (m, 1H), 3.03 (br d, J=12.2 Hz, 1H), 2.82-2.69 (m, 2H), 2.11 (s, 1H), 1.96-1.80 (m, 3H), 1.67 (br s, 1H).

### Example 685

### Preparation of (R)—N-(1-(4-amino-7-(pyrimidin-5-yl)pyrrolo[2,1-f][1,2,4]triazin-5-yl)piperidin-3-yl)-5-cyano-1H-indazole-3-carboxamide

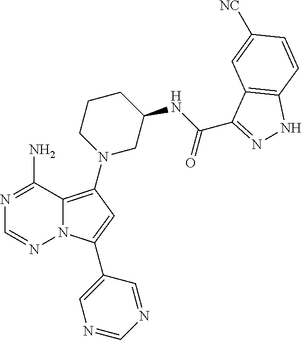 [SMILES: N#Cc1ccc2[nH]nc(C(=O)N[C@@H]3CCCN(c4cc(-c5cncnc5)n5ncnc(N)c45)C3)c2c1]

### Example 685A. N—((R)-1-(4-amino-7-(pyrimidin-5-yl)pyrrolo[2,1-f][1,2,4]triazin-5-yl)piperidin-3-yl)-5-cyano-1-(tetrahydro-2H-pyran-2-yl)-1H-indazole-3-carboxamide

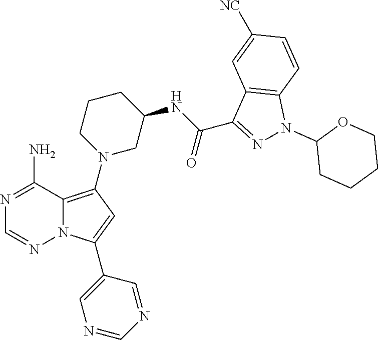 [SMILES: N#Cc1ccc2c(c1)c(C(=O)N[C@@H]1CCCN(c3cc(-c4cncnc4)n4ncnc(N)c34)C1)nn2C1CCCCO1]

Analogous to the procedure for the preparation of Example 276, reaction of Example 684D and Intermediate 12 to get crude 0.09 g of Example 685A as a brown gummy solid. MS: [M+H]⁺=564.2.

### Example 685

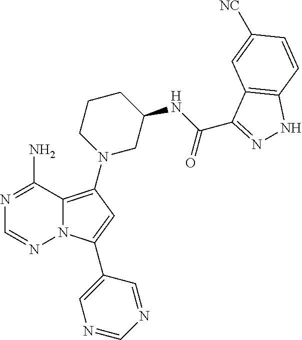 [SMILES: N#Cc1ccc2[nH]nc(C(=O)N[C@@H]3CCCN(c4cc(-c5cncnc5)n5ncnc(N)c45)C3)c2c1]

Analogous to the procedure for the preparation of Example 684, reaction of Example 685A and TFA afforded Example 685 (1.4 mg, 4% yield). LCMS Method E: RT=1.595 min, [M+H]⁺=480.2; LCMS Method F: RT=1.333 min, [M+H]⁺=480.2; ¹H NMR (400 MHz, DMSO-d₆) δ ppm 13.39 (s, 1H), 10.42 (s, 1H), 8.34 (d, J=1.8 Hz, 1H), 8.27-8.17 (m, 1H), 8.08-7.98 (m, 1H), 7.85 (s, 1H), 7.16 (dd, J=1.3, 8.5 Hz, 1H), 6.76-6.68 (m, 2H), 6.60-6.45 (m, 1H), 4.61-4.48 (m, 2H), 4.33-4.14 (m, 3H), 1.99-1.74 (m, 2H), 1.69-1.47 (m, 2H).

### Example 686

### Preparation of methyl (R)-3-(4-amino-5-(3-(5-fluoro-3-(piperidin-4-yloxy)benzo[b]thiophene-2-carboxamido)piperidin-1-yl)pyrrolo[2,1-f][1,2,4]triazin-7-yl)-2,5-dihydro-1H-pyrrole-1-carboxylate

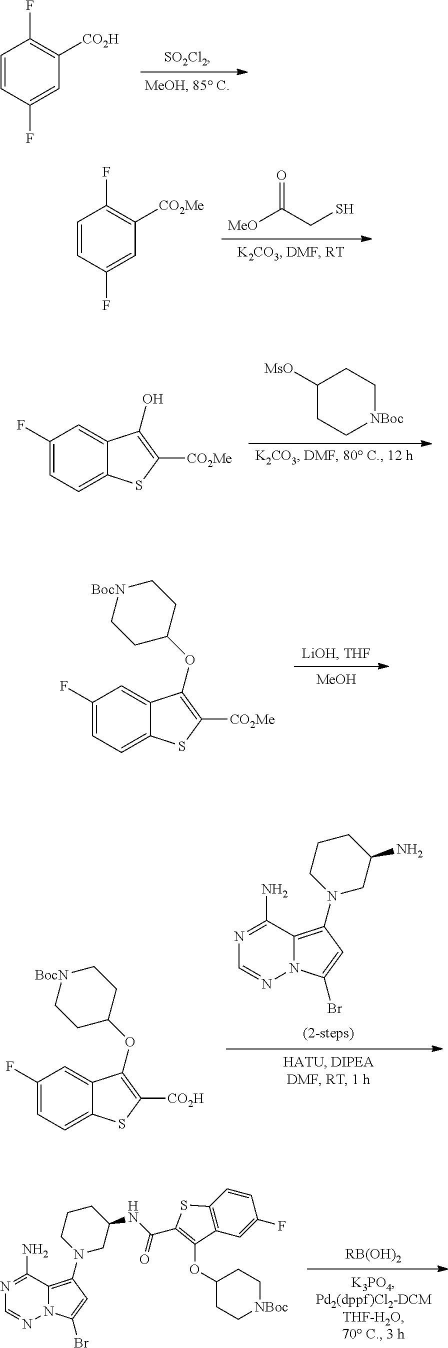

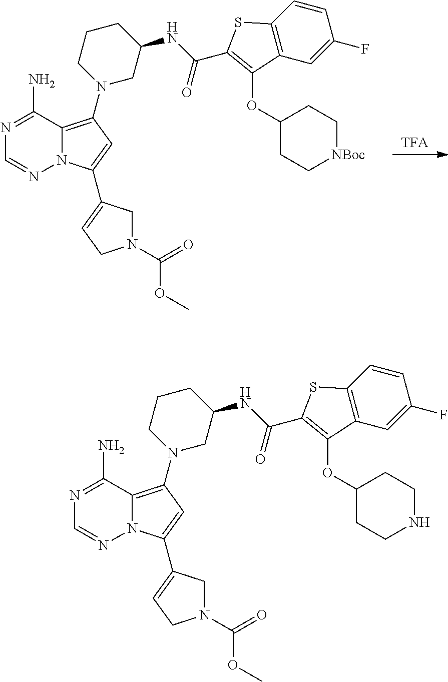 [SMILES: COC(=O)N1CC=C(c2cc(N3CCC[C@@H](NC(=O)c4sc5ccc(F)cc5c4OC4CCN(C(=O)OC(C)(C)C)CC4)C3)c3c(N)ncnn23)C1 ; COC(=O)N1CC=C(c2cc(N3CCC[C@@H](NC(=O)c4sc5ccc(F)cc5c4OC4CCNCC4)C3)c3c(N)ncnn23)C1 ; "TFA"]

### Example 686A. Methyl 5-fluoro-3-hydroxybenzo[b]thiophene-2-carboxylate

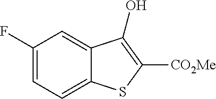 [SMILES: COC(=O)c1sc2ccc(F)cc2c1O]

To a stirred solution of methyl 2,5-difluorobenzoate (0.9 g, 5.23 mmol) in DMF (10 mL), was added K₂CO₃ (1.445 g, 10.46 mmol) at 0° C. The reaction mixture was stirred at it for 15 minutes and then methyl 2-mercaptoacetate (0.701 mL, 7.84 mmol) was added. The mixture was stirred at it for 16 h. Upon addition of ice water, the solid precipitated was filtered and dried to get Example 686A (0.5 g, 42% yield) as an off-white solid. MS: [M−H]⁺=225.0.

### Example 686B. tert-butyl 4-((5-fluoro-2-(methoxycarbonyl)benzo[b]thiophen-3-yl)oxy)piperidine-1-carboxylate

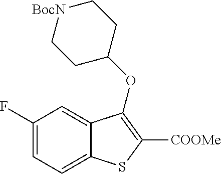 [SMILES: COC(=O)c1sc2ccc(F)cc2c1OC1CCN(C(=O)OC(C)(C)C)CC1]

Analogous to the procedure for the preparation of Example 651A, reaction of Example 686A (651 mg, 2.79 mmol) and tert-butyl 4-((methylsulfonyl)oxy)piperidine-1-carboxylate (0.617 g, 2.21 mmol) afforded Example 686B (0.6 g, 66% yield) as an off-white solid. MS: [M−Boc+2H]⁺=310.0; ¹H NMR (300 MHz, CDCl₃) δ ppm 7.64-7.72 (m, 1H), 7.46-7.55 (m, 1H), 7.23-7.25 (m, 1H), 4.66-4.75 (m, 1H), 3.91-3.94 (s, 3H), 3.07-3.29 (m, 4H), 1.73-1.91 (m, 4H), 1.43-1.48 ppm (m, 9H).

### Example 686C. 3-((1-(tert-butoxycarbonyl)piperidin-4-yl)oxy)-5-fluorobenzo[b]thiophene-2-carboxylic acid, lithium salt

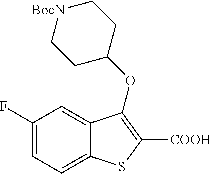 [SMILES: CC(C)(C)OC(=O)N1CCC(Oc2c(C(=O)O)sc3ccc(F)cc23)CC1]

To a stirred solution of Example 686B (0.1 g, 0.244 mmol) in THF (2 mL) and water (2 mL), was added LiOH (0.023 g, 0.977 mmol) at rt. The reaction mixture was stirred at rt for 6 h and concentrated to afford Example 686C as lithium salt (95 mg, 97% yield). MS: [M−Boc+2H]⁺=296.0.

### Example 686D. Preparation of tert-butyl (R)-4-((2-((1-(4-amino-7-bromopyrrolo[2,1-f][1,2,4]triazin-5-yl)piperidin-3-yl)carbamoyl)-5-fluorobenzo[b]thiophen-3-yl)oxy)piperidine-1-carboxylate

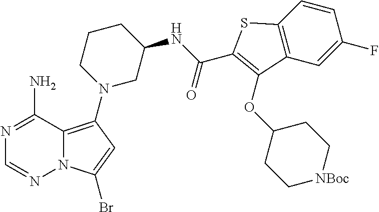 [SMILES: CC(C)(C)OC(=O)N1CCC(Oc2c(C(=O)N[C@@H]3CCCN(c4cc(Br)n5ncnc(N)c45)C3)sc3ccc(F)cc23)CC1]

Analogous to the procedure for the preparation of Example 276C, reaction of Example 686C and (R)-5-(3-aminopiperidin-1-yl)-7-bromopyrrolo[2,1-f][1,2,4]triazin-4-amine (354 mg, 1.14 mmol) afforded Example 686D (300 mg, 38% yield) as a pale yellow solid. MS: [M+H]⁺=688.2.

### Example 686E. Preparation of tert-butyl (R)-4-((2-((1-(4-amino-7-(1-(methoxycarbonyl)-2,5-dihydro-1H-pyrrol-3-yl)pyrrolo[2,1-f][1,2,4]triazin-5-yl)piperidin-3-yl)carbamoyl)-5-fluorobenzo[b]thiophen-3-yl)oxy)piperidine-1-carboxylate

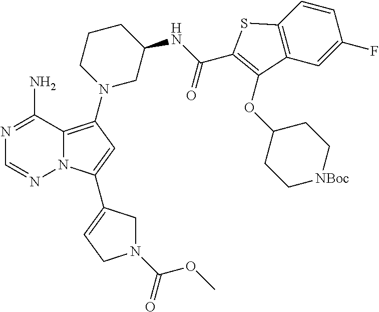 [SMILES: COC(=O)N1CC=C(c2cc(N3CCC[C@@H](NC(=O)c4sc5ccc(F)cc5c4OC4CCN(C(=O)OC(C)(C)C)CC4)C3)c3c(N)ncnn23)C1]

Analogous to the procedure for the preparation of Example 276, reaction of Example 686D and Intermediate 28 afforded Example 686E as a brown gummy solid. MS: [M+H]⁺=735.4.

### Example 686

### Preparation of methyl (R)-3-(4-amino-5-(3-(5-fluoro-3-(piperidin-4-yloxy)benzo[b]thiophene-2-carboxamido)piperidin-1-yl)pyrrolo[2,1-f][1,2,4]triazin-7-yl)-2,5-dihydro-1H-pyrrole-1-carboxylate

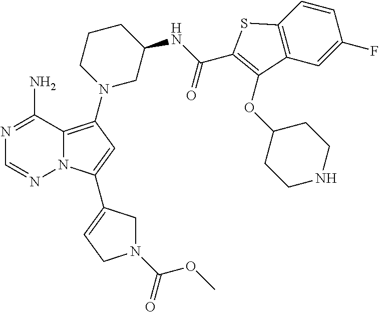 [SMILES: COC(=O)N1CC=C(c2cc(N3CCC[C@@H](NC(=O)c4sc5ccc(F)cc5c4OC4CCNCC4)C3)c3c(N)ncnn23)C1]

To a stirred solution of Example 686E (0.15 g, 0.20 mmol) in CH₂Cl₂ (5 mL), was added TFA (1 mL) at rt. The reaction mixture was stirred at rt for 12 h and concentrated to afford the crude product which was purified by preparative HPLC to get Example 686 (12 mg, 9% yield). LCMS Method E: RT=1.501 min. [M+H]⁺=635.3; LCMS Method F: RT=1.219 min, [M+H]⁺=635.3; ¹H NMR (400 MHz, DMSO-d₆) δ ppm 8.12-7.93 (m, 2H), 7.87 (s, 1H), 7.70 (dd, J=2.4, 9.2 Hz, 1H), 7.41 (dt, J=2.4, 8.9 Hz, 1H), 6.74 (d, J=2.4 Hz, 2H), 4.65-4.47 (m, 2H), 4.35-4.15 (m, 3H), 3.76 (s, 3H), 3.66 (d, J=3.7 Hz, 2H), 3.13-2.90 (m, 1H), 2.86-2.72 (m, 1H), 2.62 (br d, J=8.6 Hz, 1H), 2.13-2.04 (m, 2H), 2.02-1.80 (m, 4H), 1.74-1.47 (m, 4H).

### Example 688

### Preparation of (R)—N-(1-(4-amino-7-(pyrimidin-5-yl) pyrrolo[2,1-f][1,2,4]triazin-5-yl)piperidin-3-yl)-5-fluoro-3-(piperidin-4-yloxy)benzo[b]thiophene-2-carboxamide

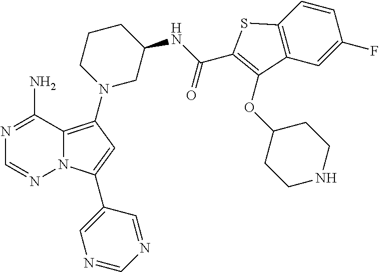 [SMILES: Nc1ncnn2c(-c3cncnc3)cc(N3CCC[C@@H](NC(=O)c4sc5ccc(F)cc5c4OC4CCNCC4)C3)c12]

### Example 688A. tert-butyl (R)-4-((2-((1-(4-amino-7-(pyrimidin-5-yl)pyrrolo[2,1-f][1,2,4]triazin-5-yl)piperidin-3-yl)carbamoyl)-5-fluorobenzo[b]thiophen-3-yl)oxy)piperidine-1-carboxylate

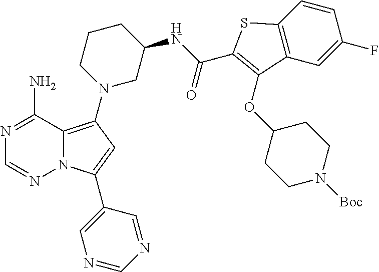 [SMILES: CC(C)(C)OC(=O)N1CCC(Oc2c(C(=O)N[C@@H]3CCCN(c4cc(-c5cncnc5)n5ncnc(N)c45)C3)sc3ccc(F)cc23)CC1]

Analogous to the procedure for the preparation of Example 276, reaction of Example 686D (100 mg, 0.145 mmol) and Intermediate 12 afforded 0.15 g of Example 688A as a brown gummy solid. MS: [M+H]⁺=688.3. Example 688 Reaction of Example 688A (0.15 g, 0.218 mmol) and TFA afforded Example 688 (11.6 mg, 8% yield). LCMS Method E: RT=1.290 min, [M+H]⁺=588.2; LCMS Method F: RT=1.290 min, [M+H]⁺=588.2; ¹H NMR (400 MHz, DMSO-d₆) δ ppm 9.48 (s, 2H), 9.10 (s, 1H), 8.62-8.35 (m, 1H), 8.06 (dd, J=4.9, 9.2 Hz, 1H), 7.94 (s, 1H), 7.72 (dd, J=2.4, 9.8 Hz, 1H), 7.42 (dt, J=2.4, 8.9 Hz, 1H), 7.31 (s, 1H), 7.24 (s, 1H), 7.12 (s, 1H), 6.99 (s, 1H), 4.73-4.65 (m, 4H), 4.24 (br s, 1H), 3.05-2.75 (m, 4H), 2.27-2.07 (m, 4H), 2.03-1.79 (m, 4H), 1.64 (br s, 1H).

### Example 689

### Preparation of methyl (R)-3-(4-amino-5-(3-(5-amino-1H-indazole-3-carboxamido)piperidin-1-yl)pyrrolo[2,1-f][1,2,4]triazin-7-yl)-2,5-dihydro-1H-pyrrole-1-carboxylate

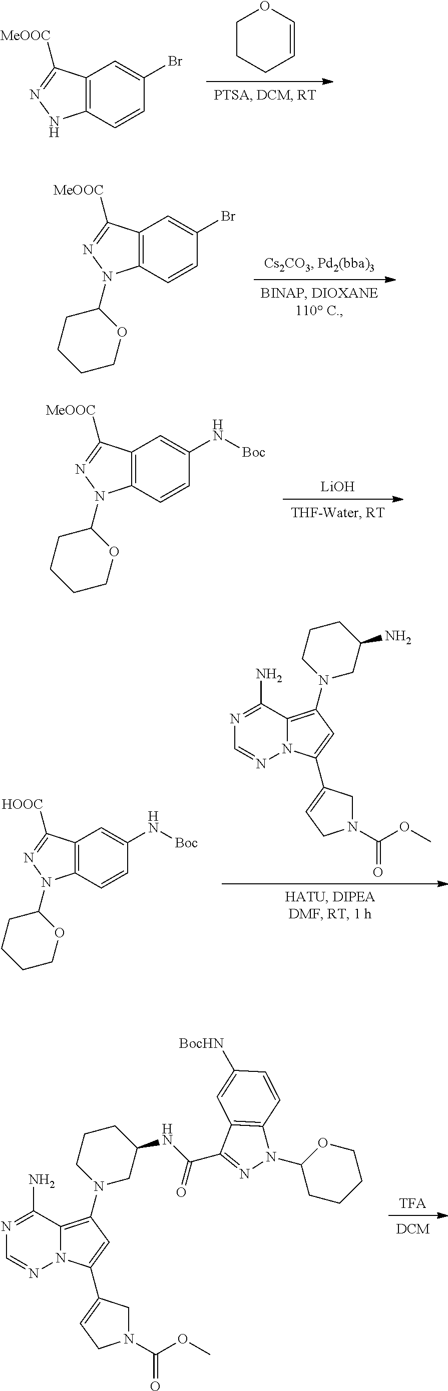

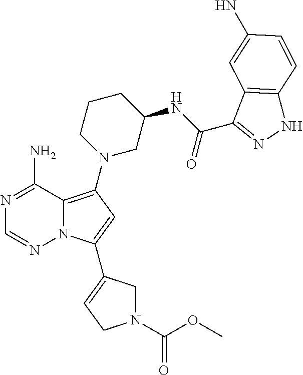 [SMILES: COC(=O)N1CC=C(c2cc(N3CCC[C@@H](NC(=O)c4n[nH]c5ccc(N)cc45)C3)c3c(N)ncnn23)C1]

### Example 689A. Preparation of methyl 5-bromo-1-(tetrahydro-2H-pyran-2-yl)-1H-indazole-3-carboxylate

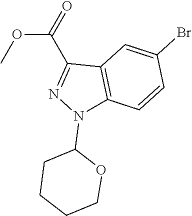 [SMILES: COC(=O)c1nn(C2CCCCO2)c2ccc(Br)cc12]

To a stirred solution of methyl 5-bromo-1H-indazole-3-carboxylate (5 g, 19.6 mmol) in DCM (20 mL), were added PTSA (0.373 g, 1.96 mmol) and 3,4-dihydro-2H-pyran (2.68 mL, 29.4 mmol) at rt. The reaction mixture was stirred at rt for 16 h. The reaction mixture was diluted with dichloromethane (100 mL) followed by the addition of 10% aq. solution of NaHCO₃ (50) and water (100 mL). The organic layer was separated, dried over anhydrous sodium sulphate, filtered and concentrated to get crude product. The crude product was purified by flash chromatography (6% ethyl acetate in pet ether) to get Example 689A (6 g, 90% yield) as a pale yellow solid. MS: [M−84+H]⁺=254.9.

### Example 689B. Methyl 5-((tert-butoxycarbonyl)amino)-1-(tetrahydro-2H-pyran-2-yl)-1H-indazole-3-carboxylate

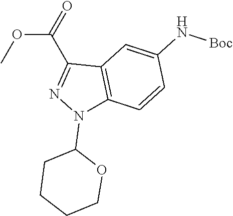 [SMILES: COC(=O)c1nn(C2CCCCO2)c2ccc(NC(=O)OC(C)(C)C)cc12]

To a stirred solution of Example 689A (0.25 g, 0.737 mmol) in 1,4-dioxane (10 mL), were added tert-butyl carbamate (0.086 g, 0.737 mmol), Cs₂CO₃ (0.720 g, 2.211 mmol), xantphos (0.021 g, 0.037 mmol) and Pd₂(dba)₃ (0.067 g, 0.074 mmol) at rt. The reaction mixture was heated at 110° C. for 16 h. After cooling, it was filtered through Celite and the filtrate was concentrated. The crude product was purified by silica gel column using 35% ethyl acetate in pet ether to get Example 689B (0.15 g, 54% yield) as a brown solid. MS: [M−H]⁺=374.2.

### Example 689C. 5-((tert-butoxycarbonyl)amino)-1-(tetrahydro-2H-pyran-2-yl)-1H-indazole-3-carboxylic acid. lithium salt

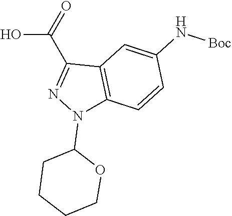 [SMILES: CC(C)(C)OC(=O)Nc1ccc2c(c1)c(C(=O)O)nn2C1CCCCO1]

To a stirred solution of Example 689B (0.09 g, 0.240 mmol) in THF (2 mL), methanol (2 mL) and water (1 mL), was added LiOH (0.011 g, 0.479 mmol) at rt. The reaction mixture was stirred at rt for 2 h and concentrated to afford Example 689C as the lithium salt (0.08 g, 91% yield) as an off-white solid. MS: [M−84+H]⁺=277.8.

### Example 689D. Methyl 3-(4-amino-5-((3R)-3-(5-((tert-butoxycarbonyl)amino)-1-(tetrahydro-2H-pyran-2-yl)-1H-indazole-3-carboxamido)piperidin-1-yl)pyrrolo[2,1-f][1,2,4]triazin-7-yl)-2,5-dihydro-1H-pyrrole-1-carboxylate

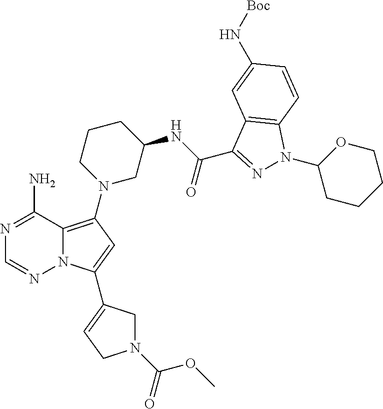 [SMILES: COC(=O)N1CC=C(c2cc(N3CCC[C@@H](NC(=O)c4nn(C5CCCCO5)c5ccc(NC(=O)OC(C)(C)C)cc45)C3)c3c(N)ncnn23)C1]

Analogous to the procedure for the preparation of Example 561C, reaction of Intermediate 31 (65.2 mg, 0.138 mmol) and Example 689C (50 mg, 0.138 mmol) gave Example 689D as a pale yellow solid. The crude compound was taken up for next the step without further purification. MS: [M+H]⁺=701.4.

### Example 689

Analogous to the procedure for the preparation of Example 684, reaction of Example 689D (0.05 g, 0.071 mmol) and TFA gave Example 689 (1.4 mg, 4% yield). LCMS Method E: RT=1.427 min, [M+H]⁺=517.2; ¹H NMR (400 MHz, DMSO-d₆) δ ppm 13.39 (s, 1H), 10.42 (s, 1H), 8.34 (d, J=1.8 Hz, 1H), 8.27-8.17 (m, 1H), 8.08-7.98 (m, 1H), 7.85 (s, 1H), 7.16 (dd, J=1.3, 8.5 Hz, 1H), 6.76-6.68 (m, 2H), 6.60-6.45 (m, 1H), 4.61-4.48 (m, 2H), 4.33-4.14 (m, 3H), 3.64 (d, J=3.5 Hz, 3H), 3.21-3.16 (m, 1H), 3.04-2.93 (m, 1H), 2.83-2.66 (m, 2H), 1.99-1.74 (m, 3H), 1.69-1.47 (m, 1H).

### Example 690

### Preparation of (R)-5-amino-N-(1-(4-amino-7-(pyrimidin-5-yl)pyrrolo[2,1-f][1,2,4]triazin-5-yl)piperidin-3-yl)-1H-indazole-3-carboxamide

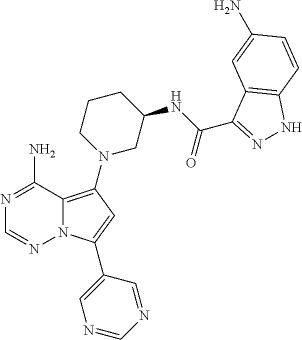 [SMILES: Nc1ccc2[nH]nc(C(=O)N[C@@H]3CCCN(c4cc(-c5cncnc5)n5ncnc(N)c45)C3)c2c1]

### Example 690A. tert-butyl (3-(((R)-1-(4-amino-7-(pyrimidin-5-yl)pyrrolo[2,1-f][1,2,4]triazin-5-yl)piperidin-3-yl)carbamoyl)-1-(tetrahydro-2H-pyran-2-yl)-1H-indazol-5-yl)carbamate

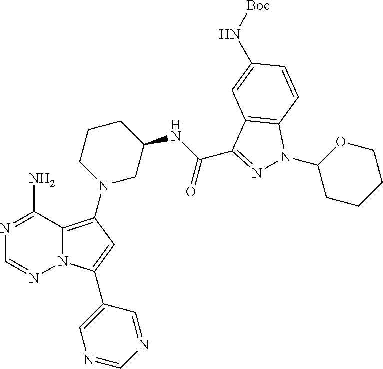 [SMILES: CC(C)(C)OC(=O)Nc1ccc2c(c1)c(C(=O)N[C@@H]1CCCN(c3cc(-c4cncnc4)n4ncnc(N)c34)C1)nn2C1CCCCO1]

Analogous to the procedure for the preparation of Example 561C, reaction of Example 689C (50 mg, 0.138 mmol) and (R)-5-(3-aminopiperidin-1-yl)-7-(pyrimidin-5-yl) pyrrolo[2,1-f][1,2,4]triazin-4-amine (58.7 mg, 0.138 mmol) afforded Example 690A (0.07 g, 77% yield) as a brown solid. MS: [M+H]⁺=654.4.

### Example 690

Analogous to the procedure for the preparation of Example 684, reaction of Example 690A and TFA gave Example 690 (15.9 mg, 52% yield). LCMS Method E: RT=1.173 min, [M+H]⁺=470.2; LCMS Method F: RT=0.860 min, [M+H]⁺=470.2; ¹H NMR (400 MHz, DMSO-d₆) δ ppm 12.78 (s, 1H), 9.47 (s, 2H), 9.08 (s, 1H), 8.07 (d, J=8.0 Hz, 2H), 7.92 (s, 1H), 7.77 (d, J=8.5 Hz, 1H), 7.30 (s, 1H), 7.04 (br s, 1H), 6.65-6.53 (m, 2H), 4.24 (br s, 1H), 4.29-4.15 (m, 1H), 3.26 (br s, 3H), 3.06 (br s, 1H), 2.84-2.68 (m, 2H), 1.96-1.76 (m, 3H), 1.66-1.65 (m, 1H), 1.70-1.55 (m, 1H).

### Example 691

### Preparation of (R)—N-(1-(4-amino-7-(1,5-dimethyl-6-oxo-1,6-dihydropyridin-3-yl)pyrrolo[2,1-f][1,2,4]triazin-5-yl)piperidin-3-yl)-5-chloro-3-(2-(dimethylamino)ethoxy)thiophene-2-carboxamide

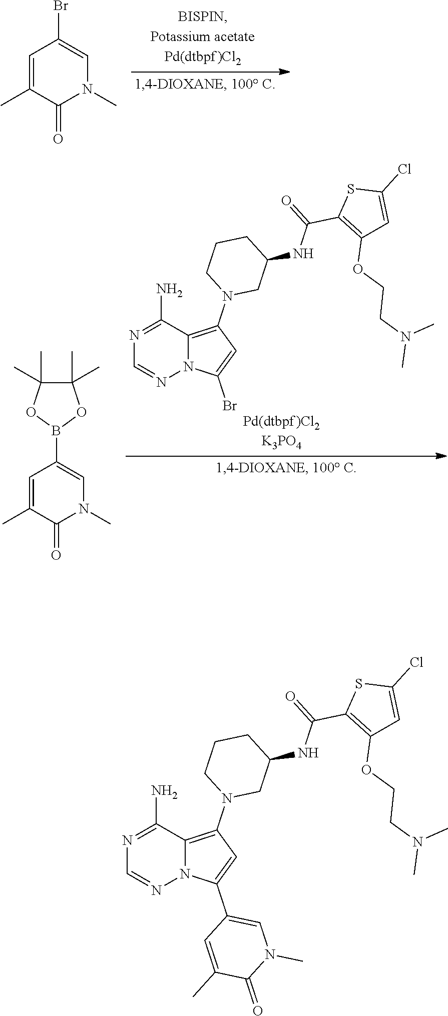

### Example 691A. 1,3-dimethyl-5-(4,4,5,5-tetramethyl-1,3,2-dioxaborolan-2-yl)pyridin-2(1H)-one

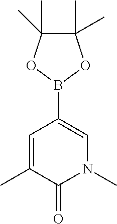

To a stirred solution of 5-bromo-1,3-dimethylpyridin-2(1H)-one (250 mg, 1.247 mmol) in 1,4-dioxane (15 mL), were added potassium acetate (304 mg, 3.09 mmol) and bis(pinacolato)diboron (377 mg, 1.495 mmol) and PdCl₂(dppf)-CH₂Cl₂ adduct (109 mg, 0.133 mmol) at it. The reaction mixture was stirred at 100° C. for 2 b. The reaction mixture was diluted with ethyl acetate (50 mL) and water (50 mL) was added. Organic layer was separated, dried over anhydrous sodium sulphate, filtered and concentrated to get crude 0.360 g of Example 691A as a brown gummy solid. MS: [M+H]⁺=250.1.

### Example 691

To a stirred solution of Example 276C (0.075 g, 0.138 mmol) in THF (10 mL) and water (2 mL), Example 691A (0.052 g, 0.207 mmol), potassium phosphate dibasic (0.060 g, 0.345 mmol) and PdCl₂(dppf)-CH₂Cl₂ adduct (5.6 mg, 6.91 μmol) were added at rt. The reaction mixture was heated at 75° C. for 18 h. The crude compound was concentrated and purified by preparative HPLC to get Example 691. LCMS Method E: RT=1.603 min, [M+H]⁺=585.2; LCMS Method F: RT=0.951 min, [M+H]⁺=585.2; ¹H NMR (400 MHz, DMSO-d₆) δ ppm 14.19 (br s, 1H), 8.94-8.80 (m, 1H), 8.49 (d, J=8.3 Hz, 1H), 7.57 (dd, J=1.2, 8.6 Hz, 1H), 7.27 (br s, 1H), 6.81 (d, J=4.9 Hz, 1H), 6.76 (br s, 2H), 4.60-4.49 (m, 3H), 4.37-4.23 (m, 3H), 3.65 (br s, 3H), 3.24-3.19 (m, 3H), 3.03 (br d, J=12.2 Hz, 1H), 2.82-2.69 (m, 3H), 2.11 (s, 3H), 1.96-1.80 (m, 3H), 1.67 (br s, 3H).

### Example 692

### Preparation of (R)—N-(1-(4-amino-7-(1-ethyl-5-methyl-6-oxo-1,6-dihydropyridin-3-yl)pyrrolo[2,1-f][1,2,4]triazin-5-yl)piperidin-3-yl)-5-chloro-3-(2-(dimethylamino)ethoxy)thiophene-2-carboxamide

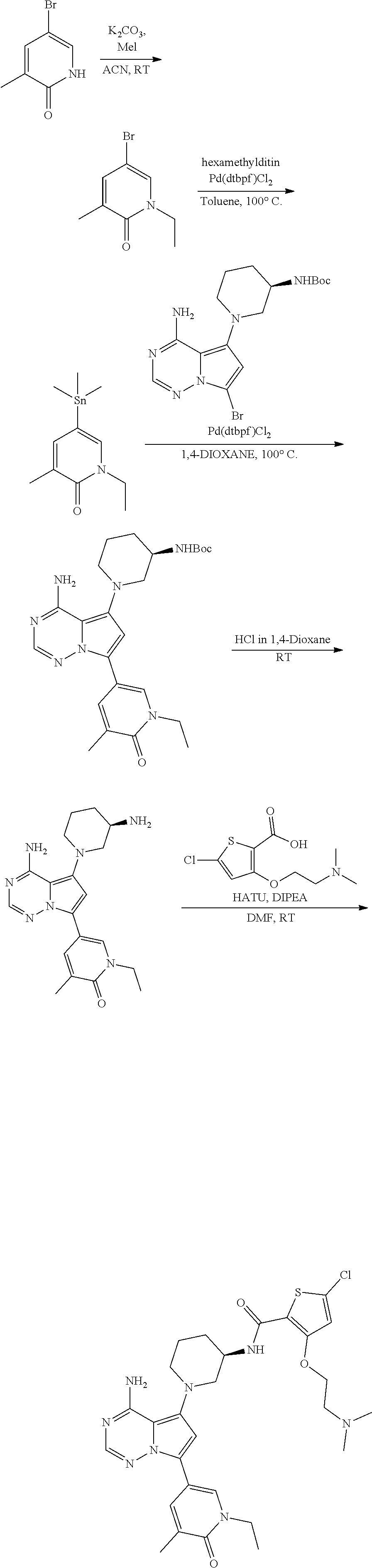

### Example 692A. 5-bromo-1-ethyl-3-methylpyridin-2(1H)-one

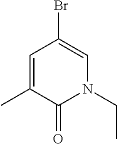

To a stirred solution of 5-bromo-3-methylpyridin-2(1H)-one (1.0 g, 5.32 mmol) in acetonitrile (20 mL), were added K₂CO₃ (1.103 g, 7.98 mmol) and iodoethane (0.830 g, 5.32 mmol) at rt. The reaction mixture was stirred at 80° C. for 16 h. The reaction mixture was filtered through Celite and filtrate was diluted with ethyl acetate (50 mL) and water (40 mL). Organic layer was separated, dried over anhydrous sodium sulphate, filtered and concentrated. The crude product was purified by flash chromatography (38% ethyl acetate in pet. ether) to get Example 692A (500 mg, 44% yield) as an off-white solid. MS: [M+H]⁺=215.9; ¹H NMR (400 MHz, DMSO-d₆, 27° C.) δ ppm 7.88 (d, J=3.5 Hz, 1H), 7.42-7.43 (m, 1H), 3.90 (q, J=7.4 Hz, 2H), 2.00 (s, 3H), 1.20 (t, J=7.0 Hz, 3H).

### Example 692B. 1-ethyl-3-methyl-5-(trimethylstannyl)pyridin-2(1H)-one

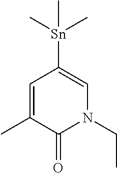

To a stirred solution of Example 692A (150 mg, 0.694 mmol) in toluene (4 mL), was added hexamethylditin (0.158 mL, 0.764 mmol) and 1 1′-bis(di-tert-butylphosphino)ferrocene dichloropalladium(II) (5.2 mg, 0.069 mmol) at rt. The reaction mixture was stirred at 100° C. for 2 h, filtered through Celite and the filtrate was concentrated to get crude Example 692B (0.2 g, 96% yield) as a brown gummy solid. MS: [M+H]⁺=302.

### Example 692C. tert-butyl (R)-(1-(4-amino-7-(1-ethyl-5-methyl-6-oxo-1,6-dihydropyridin-3-yl)pyrrolo[2,1-f][1,2,4]triazin-5-yl)piperidin-3-yl)carbamate

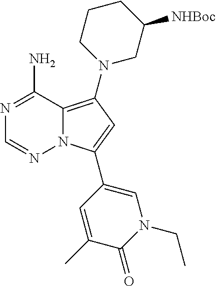 [SMILES: CCn1cc(-c2cc(N3CCC[C@@H](NC(=O)OC(C)(C)C)C3)c3c(N)ncnn23)cc(C)c1=O]

To a stirred solution of Example 692B (0.192 g, 0.467 mmol) and 1-ethyl-3-methyl-5-(trimethylstannyl)pyridin-2(1H)-one (0.200 g, 0.667 mmol) in 1,4-dioxane (4 mL), 1V′-bis(di-tert-butylphosphino)ferrocene dichloropalladium(II) (0.067 mmol) was added and reaction mixture was heated at 100° C. for 18 h. The reaction mixture was filtered through Celite and filtrate was concentrated to get 0.37 g of crude product which was purified by preparative HPLC to get Example 692C (0.09 g, 29% yield) as a brown gummy solid. MS: [M+H]⁺=468.4.

### Example 692D. (R)-5-(4-amino-5-(3-aminopiperidin-1-yl)pyrrolo[2,1-f][1,2,4]triazin-7-yl)-1-ethyl-3-methylpyridin-2(1H)-one

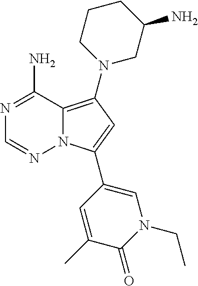 [SMILES: CCn1cc(-c2cc(N3CCC[C@@H](N)C3)c3c(N)ncnn23)cc(C)c1=O]

To a stirred solution of Example 692C in CH₂Cl₂ (5 mL), was added TFA (1 mL) at rt. The reaction mixture was stirred at rt for 2 h and concentrated to get 0.12 g of Example 692D as a brown gummy solid. MS: [M+H]⁺=368.2.

### Example 692

To a stirred solution of Example 276B in DMF (2 mL), were added Example 692D (0.05 g, 0.136 mmol), HATU (0.062 g, 0.163 mmol) and DIEA (0.071 mL, 0.408 mmol) at rt. The reaction mixture was stirred at rt for 2 h, concentrated and purified by preparative HPLC to get Example 692. LCMS Method E: RT=1.693 min, [M+H]⁺=599.2; LCMS Method F: RT=1.016 min, [M+H]⁺=599.2; ¹H NMR (400 MHz, DMSO-d₆) δ ppm 14.19 (br s, 1H), 8.94-8.80 (m, 1H), 8.49 (d, J=8.3 Hz, 1H), 7.57 (dd, J=1.2, 8.6 Hz, 1H), 7.27 (br s, 1H), 6.81 (d, J=4.9 Hz, 1H), 6.76 (br s, 2H), 4.60-4.49 (m, 3H), 4.37-4.23 (m, 3H), 3.90 (q, J=7.4 Hz, 2H), 3.24-3.19 (m, 3H), 3.03 (br d, J=12.2 Hz, 1H), 2.82-2.69 (m, 3H), 2.11 (s, 3H), 1.96-1.80 (m, 3H), 1.67 (br s, 3H).

### Example 693

### Preparation of (R)—N-(1-(4-amino-7-(1-isopropyl-5-methyl-6-oxo-1,6-dihydropyridin-3-yl)pyrrolo[2,1-f][1,2,4]triazin-5-yl)piperidin-3-yl)-5-chloro-3-(2-(dimethylamino)ethoxy)thiophene-2-carboxamide

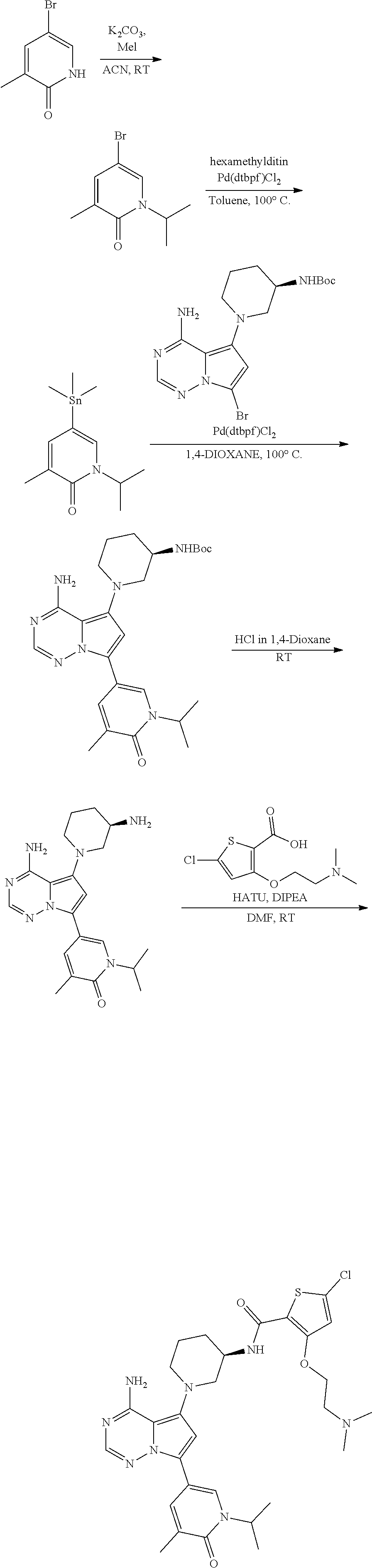

### Example 693A. 5-bromo-1-isopropyl-3-methylpyridin-2(1H)-one

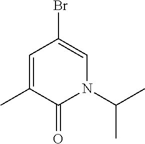

To a stirred solution of 5-bromo-3-methylpyridin-2(1H)-one (1.0 g, 5.32 mmol) in acetonitrile (20 mL), were added K₂CO₃ (1.103 g, 7.98 mmol) and 2-bromopropane (0.654 g, 5.32 mmol) at rt. The reaction mixture was stirred at 80° C. for 16 h. After cooling, mixture was diluted with ethyl acetate and water. Organic layer was separated, dried over anhydrous sodium sulphate, filtered and concentrated. The crude product was purified by silica gel column (40% ethyl acetate in pet ether) to get Example 693A (400 mg, 33% yield) as an off-white solid. MS: [M+H]⁺=232.0; ¹H NMR (400 MHz, DMSO-d₆,) δ ppm 7.81 (d, J=2.5 Hz, 1H), 7.41 (dd, J=2.5, 1.0 Hz, 1H), 5.04 (quin, J=6.8 Hz, 1H), 2.01 (s, 3H), 1.29 ppm (d, J=6.5 Hz, 6H).

### Example 693B. 1-isopropyl-3-methyl-5-(trimethylstannyl)pyridin-2(1H)-one

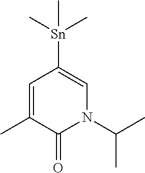

Analogous to the procedure for the preparation of Example 692B, reaction of Example 693A gave Example 693B as a brown gummy solid. MS: [M+H]⁺=316.0.

### Example 693C. tert-butyl (R)-(1-(4-amino-7-(1-isopropyl-5-methyl-6-oxo-1,6-dihydropyridin-3-yl)pyrrolo[2,1-f][1,2,4]triazin-5-yl)piperidin-3-yl)carbamate

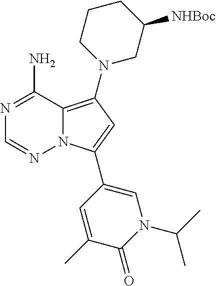 [SMILES: Cc1cc(-c2cc(N3CCC[C@@H](NC(=O)OC(C)(C)C)C3)c3c(N)ncnn23)cn(C(C)C)c1=O]

Analogous to the procedure for the preparation of Example 692C, reaction of Example 693B (150 mg, 0.365 mmol) and equivalent amount (R)-5-(3-aminopiperidin-1-yl)-7-bromopyrrolo[2,1-f][1,2,4]triazin-4-amine tert-butyl afforded Example 693C (80 mg, 46% yield) as a brown solid. MS: [M+H]⁺=482.3.

### Example 693D. Preparation of (R)-5-(4-amino-5-(3-aminopiperidin-1-yl)pyrrolo[2,1-f][1,2,4]triazin-7-yl)-1-isopropyl-3-methylpyridin-2(1H)-one, TFA

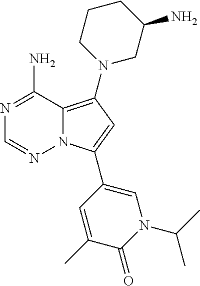 [SMILES: Cc1cc(-c2cc(N3CCC[C@@H](N)C3)c3c(N)ncnn23)cn(C(C)C)c1=O]

Analogous to the procedure for the preparation of Example 692D, reaction of Example 693C (0.08 g, 0.166 mmol) and TFA (0.128 mL, 1.66 mmol) afforded Example 693D (0.07 g, 85% yield) as a brown solid. MS: [M+H]⁺=382.3.

### Example 693

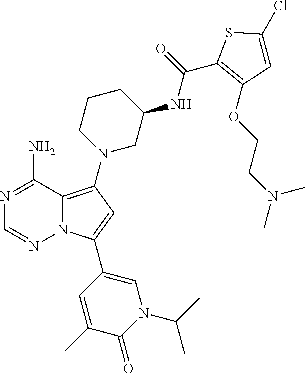 [SMILES: Cc1cc(-c2cc(N3CCC[C@@H](NC(=O)c4sc(Cl)cc4OCCN(C)C)C3)c3c(N)ncnn23)cn(C(C)C)c1=O]

Analogous to the procedure for the preparation of Example 692, reaction of Example 693D (0.05 g, 0.131 mmol) and equivalent amount of Example 276B afforded Example 693. LCMS Method E: RT=1.807 min, [M+H]⁺=613.2; LCMS Method F: RT=1.086 min, [M+H]⁺=613.3; ¹H NMR (400 MHz, DMSO-d₆) δ ppm 14.19 (br s, 1H), 8.94-8.80 (m, 1H), 8.49 (d, J=8.3 Hz, 1H), 7.57 (dd, J=1.2, 8.6 Hz, 1H), 7.27 (br s, 1H), 6.81 (d, J=4.9 Hz, 1H), 6.76 (br s, 2H), 5.04 (quin, J=6.8 Hz, 1H), 4.60-4.49 (m, 3H), 4.37-4.23 (m, 3H), 3.24-3.19 (m, 3H), 3.03 (br d, J=12.2 Hz, 1H), 2.82-2.69 (m, 3H), 2.11 (s, 3H), 1.96-1.80 (m, 3H), 1.29 ppm (d, J=6.5 Hz, 6H).

### Example 694

### Preparation of (R)—N-(1-(4-amino-7-(1-isobutyl-5-methyl-6-oxo-1,6-dihydropyridin-3-yl)pyrrolo[2,1-f][1,2,4]triazin-5-yl)piperidin-3-yl)-5-chloro-3-(2-(dimethylamino)ethoxy)thiophene-2-carboxamide

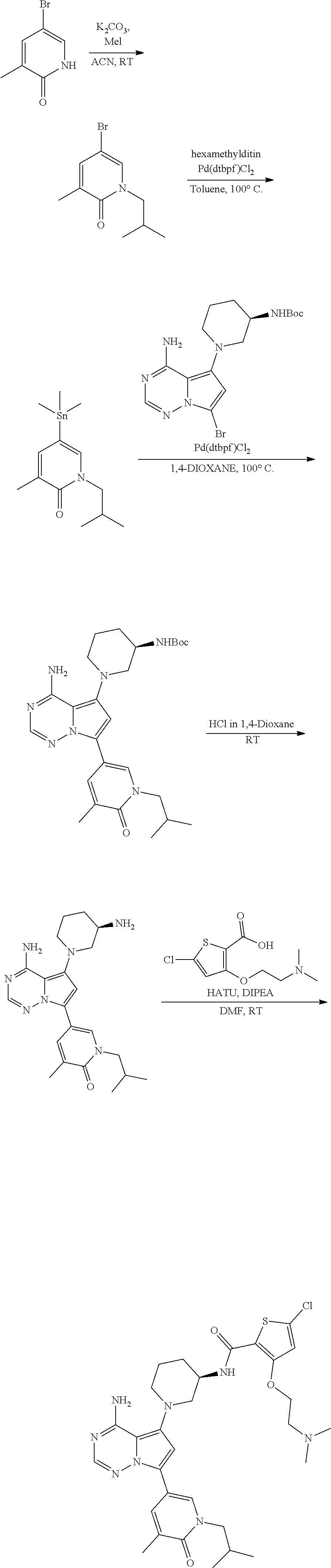

### Example 694A. 5-bromo-1-isobutyl-3-methylpyridin-2(1H)-one

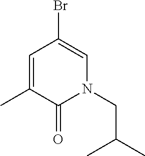

To a stirred solution of 5-bromo-3-methylpyridin-2(1H)-one (1.0 g, 5.32 mmol) in acetonitrile (20 mL), were added K₂CO₃ (1.103 g, 7.98 mmol) and 1-iodo-2-methylpropane (0.979 g, 5.32 mmol) at it. The reaction mixture was stirred at 80° C. for 16 h. After cooling, mixture was diluted with ethyl acetate (40 mL) followed by the addition of water (60 mL). Organic layer was separated, dried over anhydrous sodium sulphate, filtered and concentrated. Residue was purified by flash chromatography (45% ethyl acetate in pet-ether) to get Example 694A (250 mg, 19% yield) as a pale yellow liquid. MS: [M+H]⁺=244.0; ¹H NMR (400 MHz, DMSO-d₆, 27° C.). δ ppm 7.82 (s, 1H), 7.44 (dd, J=2.8, 1.3 Hz, 1H), 3.70 (d, J=7.5 Hz, 2H), 1.99-2.12 (m, 4H), 0.84 ppm (d, J=7.0 Hz, 6H).

### Example 694B. 1-isobutyl-3-methyl-5-(trimethylstannyl) pyridin-2(1H)-one

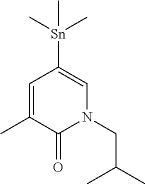 [SMILES: Cc1c[c]([Sn]([CH3])([CH3])[CH3])cn(CC(C)C)c1=O]

Analogous to the procedure for the preparation of Example 692B, reaction of Example 694A (150 mg, 0.614 mmol) afford Example 694B (180 mg, 0.549 mmol, 89% yield) as a brown gummy solid. MS: [M+H]⁺=330.0.

### Example 694C. tert-butyl (R)-(1-(4-amino-7-(1-isobutyl-5-methyl-6-oxo-1,6-dihydropyridin-3-yl)pyrrolo[2,1-f][1,2,4]triazin-5-yl)piperidin-3-yl)carbamate

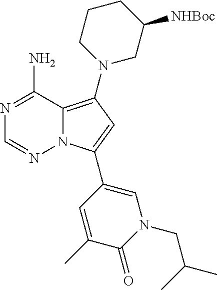 [SMILES: Cc1cc(-c2cc(N3CCC[C@@H](NC(=O)OC(C)(C)C)C3)c3c(N)ncnn23)cn(CC(C)C)c1=O]

Analogous to the procedure for the preparation of Example 692C, reaction of tert-butyl (R)-(1-(4-amino-7-bromopyrrolo[2,1-f][1,2,4]triazin-5-yl)piperidin-3-yl)carbamate (0.15 g, 0.365 mmol) and Example 694B (0.120 g, 0.365 mmol) to give Example 694C (0.09 g, 50% yield) as a brown solid. MS: [M+H]⁺=496.3.

### Example 694D. (R)-5-(4-amino-5-(3-aminopiperidin-1-yl)pyrrolo[2,1-f][1,2,4]triazin-7-yl)-1-isobutyl-3-methylpyridin-2(1H)-one.TFA

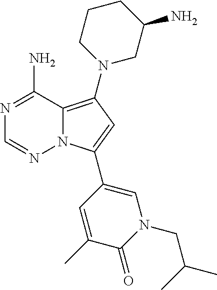 [SMILES: Cc1cc(-c2cc(N3CCC[C@@H](N)C3)c3c(N)ncnn23)cn(CC(C)C)c1=O]

To a stirred solution of Example 694C (0.08 g, 0.161 mmol) in CH₂Cl₂ (5 mL), was added TFA (1 mL) at it. The reaction mixture was stirred at it for 2 h and concentrated to get Example 694D (0.06 g, 73% yield) as a brown solid. MS: [M+H]⁺=396.3.

### Example 694

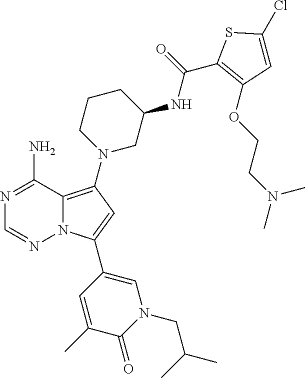 [SMILES: Cc1cc(-c2cc(N3CCC[C@@H](NC(=O)c4sc(Cl)cc4OCCN(C)C)C3)c3c(N)ncnn23)cn(CC(C)C)c1=O]

To a stirred solution of 5-chloro-3-(2-(dimethylamino)ethoxy)thiophene-2-carboxylic acid (0.038 g, 0.152 mmol) in DMF (2 mL), were added Example 694D (0.05 g, 0.126 mmol) and HATU (0.058 g, 0.152 mmol) at rt. The reaction mixture was stirred for 30 minutes, followed by the addition of DIEA (0.066 mL, 0.379 mmol) at rt and continued stirring at rt for 12 h. The mixture was concentrated and the residue was purified by preparative HPLC to get Example 694 (31.0 mg, 35% yield). LCMS Method E: RT=1.923 min, [M+H]⁺=627.2; LCMS Method F: RT=1.170 min, [M+H]⁺=627.2; ¹H NMR (400 MHz, DMSO-d₆) δ ppm 14.19 (br s, 1H), 8.94-8.80 (m, 1H), 8.49 (d, J=8.3 Hz, 1H), 7.57 (dd, J=1.2, 8.6 Hz, 1H), 7.27 (br s, 1H), 6.81 (d, J=4.9 Hz, 1H), 6.76 (br s, 2H), 4.60-4.49 (m, 3H), 4.37-4.23 (m, 3H), 3.70 (d, J=7.5 Hz, 2H), 3.24-3.19 (m, 3H), 3.03 (br d, J=12.2 Hz, 1H), 2.82-2.69 (m, 3H), 2.11 (s, 3H), 1.96-1.80 (m, 4H), 0.84 ppm (d, J=7.0 Hz, 6H).

### Example 695

### (R)—N-(1-(4-amino-7-(pyridin-4-ylethynyl)pyrrolo[2,1-f][1,2,4]triazin-5-yl)piperidin-3-yl)-5-chlorothiophene-2-carboxamide, TFA salt

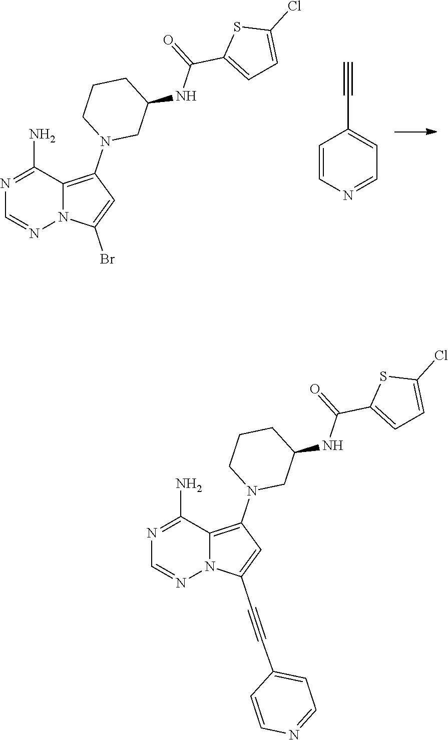

Into the reaction vessel containing Example 203C (12 mg, 0.021 mmol) was added copper(I) iodide (0.802 mg, 4.21 μmol), bis(triphenylphosphine)palladium(II) chloride (3.0 mg, 4.2 μmol), triphenylphosphine (2.2 mg, 8.4 μmol), 4-ethynylpyridine (21.7 mg, 0.211 mmol), DMF (0.5 mL) and diethylamine (0.50 mL, 4.79 mmol). The reaction mixture was degassed by bubbling N2 for 10 min, sealed, and stirred at 120° C. for 25 min under microwave. After cooled to rt, the reaction was concentrated and subjected to preparative HPLC purification to produce Example 695. LCMS Method B: RT=1.38 min, [M+H]⁺=478; ¹H NMR (DMSO-d₆, 500 MHz) δ 8.48 (br d, 1H, J=7.9 Hz), 8.01 (br s, 1H), 7.7-7.8 (m, 3H), 7.1-7.3 (m, 2H), 7.09 (s, 1H), 4.17 (br d, 1, J=8.5 Hz), 3.1-3.3 (m, 1H), 3.0-3.1 (m, 1H), 2.6-2.8 (m, 2H), 1.8-2.0 (m, 3H), 1.4-1.6 (m, 1H).

### Example 696

### (R)—N-(1-(4-amino-7-cyanopyrrolo[2,1-f][1,2,4]triazin-5-yl)piperidin-3-yl)-5-cyanothiophene-2-carboxamide

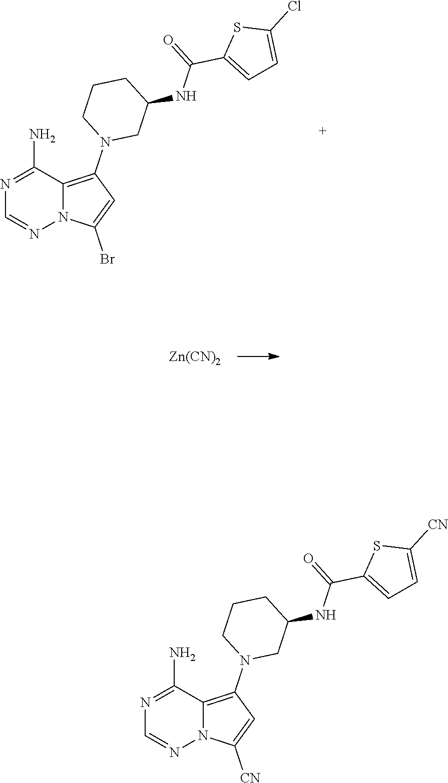 [SMILES: Nc1ncnn2c(Br)cc(N3CCC[C@@H](NC(=O)c4ccc(Cl)s4)C3)c12 ; "Zn(CN)2" ; N#Cc1ccc(C(=O)N[C@@H]2CCCN(c3cc(C#N)n4ncnc(N)c34)C2)s1]

Into the reaction vessel was added dicyanozinc (30.9 mg, 0.263 mmol), Example 203C (15 mg, 0.026 mmol), Pd₂(dba)₃ (4.8 mg, 5.3 μmol), RUPHOS (4.9 mg, 10.5 μmol), and DMF (2 mL). After bubbling N₂ for 5 min, the mixture was heated at 100° C. for 40 min, diluted with EtOAc and wash with Sat NaHCO₃. The organic phase was dried over Na₂SO₄, concentrated and subjected to prep HPLC purification to produce Example 6%. LCMS Method B: RT=1.49 min, [M+H]⁺=393; ¹H NMR (DMSO-d₆, 500 MHz) δ 8.79 (br d, 1H, J=7.6 Hz), 8.4-8.5 (m, 1H), 7.9-8.0 (m, 2H), 7.8-7.9 (m, 1H), 7.33 (s, 1H), 7.10 (br s, 1H), 4.1-4.2 (m, 1H), 3.1-3.2 (m, 1H), 2.9-3.0 (m, 1H), 2.6-2.8 (m, 2H), 1.7-2.0 (m, 3H), 1.4-1.6 (m, 1H).

### Example 700

### (R)—N-(1-(7-(1-acetyl-2,5-dihydro-1H-pyrrol-3-yl)-4-aminopyrrolo[2,1-f][1,2,4]triazin-5-yl)piperidin-3-yl)-6-(2-morpholinoethoxy)picolinamide

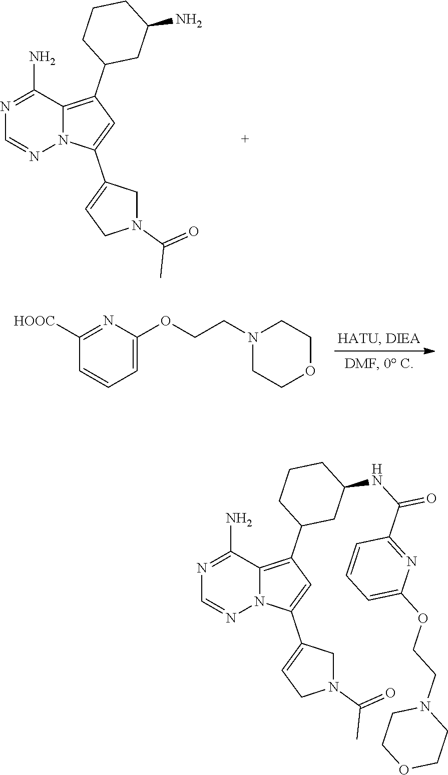

To a solution of Intermediate 32 (15 mg, 0.026 mmol) in DMF (1 mL) was added Intermediate 35 (13 mg, 0.026 mmol) followed by the addition of DIEA (0.046 mL, 0.26 mmol) and HATU (15 mg, 0.040 mmol) at 0° C. The reaction was stirred under N₂ at 0° C. for 1 h. Reverse phase chromatography provided Example 700 (6.3 mg, 41%). LC-MS Method B: ESI m/z 576 (M+H)⁺, RT=1.04 min; ¹H NMR (500 MHz, DMSO-d₆) δ 8.30 (br s, 1H), 7.91-7.83 (m, 2H), 7.62 (br d, J=7.0 Hz, 1H), 7.02 (br d, J=8.5 Hz, 1H), 6.77 (br d, J=13.7 Hz, 1H), 6.71 (br d, J=11.6 Hz, 1H), 4.69 (br s, 1H), 4.60-4.41 (m, 4H), 4.27 (br s, 1H), 4.23-4.13 (m, 1H), 3.46 (br d, J=1.8 Hz, 1H), 3.21-3.12 (m, 1H), 3.02-2.93 (m, 1H), 2.90-2.74 (m, 1H), 2.68 (br t, J=5.2 Hz, 2H), 2.55 (s, 5H), 2.45 (br s, 3H), 2.13-1.98 (m, 3H), 1.95-1.76 (m, 3H), 1.72-1.57 (m, 1H).

The following Examples in Table 54 were prepared using the similar procedure as described in Example 700 by coupling Intermediate 32 with the appropriate acids, prepared as intermediates described above, or obtained from commercial sources.

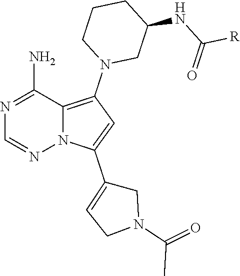

TABLE 54

| | | | | HPLC | |
|---|---|---|---|---|---|
| | | | | Meth- | |
| Ex- | | | LCMS | od, | |
| am- | | | (M + | RT | |
| ple | R | Name | H)⁺ | (min.) | ¹H NMR |
| 701 | 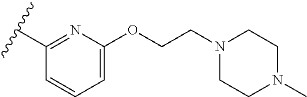 [SMILES: *c1cccc(OCCN2CCN(C)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-(2-(4- methylpiperazin-1- yl)ethoxy)picolinamide | 589 | B: 0.94 | (500 MHz, DMSO-d₆) δ 8.31- 8.16 (m, 1H), 7.99 (d, J = 1.7 Hz, 1H), 7.90 (t, J = 7.8 Hz, 1H), 7.65 (d, J = 7.2 Hz, 1H), 7.04 (d, J = 8.3 Hz, 1H), 6.86-6.74 (m, 2H), 4.70 (br s, 1H), 4.67-4.61 (m, 1H), 4.60-4.54 (m, 1H), 4.50 |
| | | | | | (br d, J = 11.1 Hz, 2H), 4.34- |
| | | | | | 4.10 (m, 3H), 3.25-3.19 (m, 1H), |
| | | | | | 3.18 (s, 4H), 3.10-2.94 (m, |
| | | | | | 3H), 2.86-2.63 (m, 6H), 2.02 (s, |
| | | | | | 3H), 1.97-1.80 (m, 4H), 1.63 |
| | | | | | (br d, J = 4.2 Hz, 1H) |
| 702 | 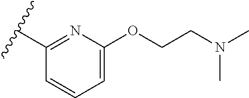 [SMILES: *c1cccc(OCCN(C)C)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-(2- (dimethylamino)ethoxy) picolinamide | 534 | B: 0.96 | (500 MHz, DMSO-d₆) δ 9.65 (br s, 1H), 8.32-8.25 (m, 1H), 8.00-7.91 (m, 2H), 7.70 (d, J = 7.4 Hz, 1H), 7.11-7.04 (m, 1H), 6.83-6.78 (m, 1H), 6.76 (d, J = 15.0 Hz, 1H), 4.83-4,67 (m, 3H), 4.53-4.46 (m, 2H), 4.29- |
| | | | | | 4.22 (m, 2H), 3.54 (br d, J = 3.7 |
| | | | | | Hz, 2H), 3.18 (s, 3H), 2.89 (br d, |
| | | | | | J = 2.6 Hz, 6H), 2.82-2.71 (m, |
| | | | | | 2H), 2.01 (s, 3H), 1.94-1.82 |
| | | | | | (m, 3H), 1.68-1.58 (m, 1H) |
| 703 | 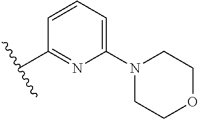 [SMILES: *c1cccc(N2CCOCC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6- morpholinopicolinamide | 532 | B: 1.37 | (500 MHz, DMSO-d₆) δ 8.23 (br s, 1H), 7.87 (d, J = 1.9 Hz, 1H), 7.71 (t. J = 7.8 Hz, 1H), 7.34 (d, J = 7.2 Hz, 1H), 7.03 (d, J = 8.5 Hz, 1H), 6.77 (br d, J = 12.5 Hz, 1H), 6.71 (d, J = 12.4 Hz, 1H), 4.68 (br d, J = 0.9 Hz, 1H), 4.48 (br d, J = 12.2 Hz, 2H), 4.27 (br d, |
| | | | | | J = 1.5 Hz, 1H), 4.22-4.12 (m, |
| | | | | | 1H), 3.72 (br t, J = 4.4 Hz, 3H), |
| | | | | | 3.52 (br d, J = 3.4 Hz, 1H), 3.15 (br |
| | | | | | dd, J = 10.2, 4.2 Hz, 1H), 3,03- |
| | | | | | 2.92 (m, 1H), 2.89-2.53 (m, |
| | | | | | 6H), 2.05, 2.01 (s, 3H), 1.95- |
| | | | | | 1.76 (m, 3H), 1.73-1.57 (m, 1H) |
| 704 | 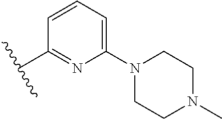 [SMILES: *c1cccc(N2CCN(C)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-(4- methylpiperazin-1- yl)picolinamide | 545 | B: 1.00 | (500 MHz, DMSO-d₆) δ 9.76 (br d, J = 2.8 Hz, 1H), 8.54 (br s, 1H), 8.23 (br dd, J = 8.3, 2.6 Hz, 1H), 7.98 (d, J = 1.7 Hz, 1H), 7.82-7.73 (m, 1H), 7.40 (d, J = 7.4 Hz, 1H), 7.15 (d, J = 8.6 Hz, 1H), 6.85-6.75 (m, 2H), 4.70 (br s, 1H), 4.61-4.45 (m, 4H), |
| | | | | | 4.33-4.18 (m, 2H), 3.54 (br d, J = |
| | | | | | 11.0 Hz, 2H), 3.21-3.03 (m, |
| | | | | | 6H), 2.88 (br s, 3H), 2.84-2.68 |
| | | | | | (m, 2H), 2.05, 2.01 (singlets, |
| | | | | | 3H), 1.96-1.79 (m, 3H), 1.71-1.56 |
| | | | | | (m, 1H) |
| 705 | 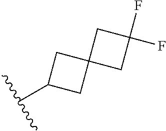 [SMILES: *C1CC2(C1)CC(F)(F)C2] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6,6- difluorospiro[3.3]heptane- 2-carboxamide | 500 | B: 1.30 | (500 MHz, DMSO-d₆) δ 8.44 (br s, 1H), 7.95 (s, 1H), 7.81 (br d, J = 4.6 Hz, 1H), 7.19 (br s, 1H), 6.84-6.72 (m, 2H), 4.70 (br d, J = 1.8 Hz, 1H), 4.49 (br dd, J = 14.3, 2.2 Hz, 2H), 4.28 (br d, J = 2.6 Hz, 1H), 4.02-3.92 (m, 1H), 3.09-3.01 (m, 1H), 2.99- 2.89 (m, 2H), 2.72 (br dd, J = 11.4, |
| | | | | | 9.0 Hz, 1H), 2.66-2.59 (m, |
| | | | | | 2H), 2.48 (m, 3H), 2.27-2.20 (m, |
| | | | | | 2H), 2.19-2.12 (m, 2H), 2.05 |
| | | | | | and 2.01 (s, 3H), 1.91-1.71 |
| | | | | | (m, 3H), 1.35 (m, 1H) |
| 706 | 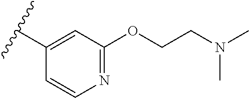 [SMILES: *c1ccnc(OCCN(C)C)c1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2-(2- (dimethylamino)ethoxy) isonicotinamide | 534 | B: 0.99 | (500 MHz, DMSO-d₆) δ 9.55 (br s, 1H), 8.63 (br d, J = 7.6 Hz, 1H), 8.53 (br s, 1H), 8.32 (d, J = 5.3 Hz, 1H), 7.97 (d, J = 1.4 Hz, 1H), 7.43 (dd, J = 5.3, 1.0 Hz, 1H), 7.25 (s, 1H), 6.84-6.75 (m, 2H), 4.70 (br s, 1H), 4.65-4.59 (m, |
| | | | | | 2H), 4.50 (br d, J = 12.8 Hz, |
| | | | | | 2H), 4.28 (br s, 1H), 4.25-4.15 (m, |
| | | | | | 1H), 3.53 (br d, J = 4.4 Hz, |
| | | | | | 2H), 3.24-3.17 (m, 1H), 3.07- |
| | | | | | 2.99 (m, 1H), 2.88 and 2.87 (s, |
| | | | | | 6H), 2.75-2.63 (m, 2H), 2.05 and |
| | | | | | 2.02 (s, 3H), 1.95-1.81 (m, |
| | | | | | 3H), 1.59-1.47 (m, 1H) |
| 707 | 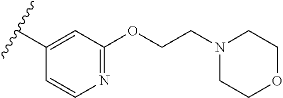 [SMILES: *c1ccnc(OCCN2CCOCC2)c1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2-(2- morpholinoethoxy) isonicotinamide | 576 | B: 0.99 | (500 MHz, DMSO-d₆) δ 9.88 (br s, 1H), 8.63 (br d, J = 7.8 Hz, 1H), 8.32 (d, J = 5.3 Hz, 1H), 7.96 (d, J = 1.0 Hz, 1H), 7.46-7.40 (m, 1H), 7.26 (s, 1H), 6.84-6.73 (m, 2H), 4.70 (br s, 1H), 4.67- 4.62 (m, 2H), 4.54-4.46 (m, 2H), |
| | | | | | 4.28 (br s, 1H), 4.25-4.15 (m, |
| | | | | | 1H), 3.98 (br s, 2H), 3.75-3.64 |
| | | | | | (m, 2H), 3.63-3.47 (m, 4H), |
| | | | | | 3.28-3.17 (m, 3H), 3.07-3.00 |
| | | | | | (m, 1H), 2.75-2.62 (m, 2H), |
| | | | | | 2.05, 2.02 (s, 3H), 1.96-1.81 |
| | | | | | (m, 3H), 1.60-1.50 (m, 1H) |
| 708 | 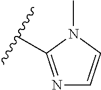 [SMILES: *c1nccn1C] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-1- methyl-1H-imidazole-2- carboxamide | 450 | B: 1.05 | (500 MHz, DMSO-d₆) δ 8.27 (br t, J = 7.9 Hz, 1H), 7.87 (d, J = 2.7 Hz, 1H), 7.34 (s, 1H), 6,98 (s, 1H), 6.77 (br d, J = 14,6 Hz, 1H), 6.71 (d, J = 13.4 Hz, 1H), 4.69 (br s, 1H), 4.48 (br d, J = 14.0 Hz, 2H), 4.31-4.22 (m, 1H), 4.11 |
| | | | | | (br s, 1H), 3.93 (s, 3H), 3.19- |
| | | | | | 3.08 (m, 1H), 3.01-2.91 (m, 1H), |
| | | | | | 2.85-2.59 (m, 2H), 2.06 and |
| | | | | | 2.01 (s, 3H), 1.90-1.71 |
| | | | | | (m, 3H), 1.61 (br s, 1H) |
| 709 | 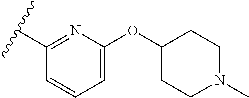 [SMILES: *c1cccc(OC2CCN(C)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-((1- methylpiperidin-4- yl)oxy)picolinamide | 560 | B: 1.07 | (500 MHz, DMSO-d₆) δ 8.26- 8.11 (m, 1H), 8.00-7.85 (m, 2H), 7.65 (br d, J = 7.0 Hz, 1H), 7.06 (br d, J = 8.9 Hz, 1H), 6.80 (br d, J = 8.5 Hz, 1H), 6.75 (br d, J = 10.7 Hz, 1H), 5.57 (br s, 1H), 4.70 (br s, 1H), 4.49 (br d, J = |
| | | | | | 10.7 Hz, 2H), 4.28 (br d, J = 0.6 Hz, |
| | | | | | 1H), 4.22 (br d, J = 12.2 Hz, 1H), |
| | | | | | 3.49-3.30 (m, 1H), 3.28-3.10 |
| | | | | | (m, 2H), 3.07-2.94 (m, 1H), 2.84 |
| | | | | | (br s, 4H), 2.34 (br dd, J = 12.4, |
| | | | | | 2.9 Hz, 1H), 2.22-2.09 (m, 1H), |
| | | | | | 2.04 (br d, J = 19.5 Hz, 3H), |
| | | | | | 1.97-1.77 (m, 3H), 1.75-1.59 (m, 1H) |
| 710 | 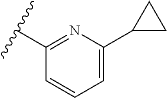 [SMILES: *c1cccc(C2CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6- cyclopropylpicolinamide, | 487 | B: 1.45 | (500 MHz, DMSO-d₆) δ 8.31 (br d, J = 7.9 Hz, 1H), 7.99 (s, 1H), 7.86-7.79 (m, 1H), 7.79-7.72 (m, 1H), 7.44 (d, J = 7.6 Hz, 1H), 6,89-6.77 (m, 2H), 4.70 (br s, 1H), 4.51 (br s, 1H), 4.48 (br s, 1H), 4.28 (br s, 1H), 4.26-4.16 (m, |
| | | | | | 1H), 3.20 (br d, J = 11.0 Hz, |
| | | | | | 1H), 3.04 (br dd, J = 8.2, 0.9 Hz, |
| | | | | | 1H), 2.88-2.81 (m, 1H), 2.80- |
| | | | | | 2.71 (m, 1H), 2.18 (quin, J = 6.3 Hz, |
| | | | | | 1H), 2.05, 2.01 (s, 3H), 1.96- |
| | | | | | 1.76 (m, 3H), 1.69-1.55 (m, |
| | | | | | 1H), 1.09-0.95 (m, 4H) |
| 711 | 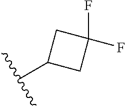 [SMILES: *C1CC(F)(F)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-3,3- difluorocyclobutane-1- carboxamide | 460 | B: 1.19 | (500 MHz, DMSO-d₆) δ 8.08 (br s, 1H), 7.80 (d, J = 1.2 Hz, 1H), 6.70 (br d, J = 13.7 Hz, 1H), 6.63 (d, J = 12.5 Hz, 1H), 4.62 (br s, 1H), 4.42 (br s, 1H), 4.40 (br s, 1H), 4.20 (br s, 1H), 3.96-3.81 (m, 1H), 3,40-2.90 (m, 4H), 2.88- |
| | | | | | 2.77 (m, 1H), 2.71-2.55 (m, |
| | | | | | 4H), 1.99, 1.94 (s, 3H), 1.87- |
| | | | | | 1.56 (m, 3H), 1.42-1.19 (m, 1H) |
| 712 | 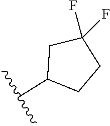 [SMILES: *C1CCC(F)(F)C1] | N-((R)-1-(7-(1-acetyl-2,5- dihydro-1H-pyrrol-3-yl)- 4-aminopyrrolo[2,1- f][1,2,4]triazin-5- yl)piperidin-3-yl)-3,3- difluorocyclopentane-1- carboxamide | 474 | B: 1.23 | (500 MHz, DMSO-d₆) δ 8.06 (br s, 1H), 7.86 (s, 1H), 6.77 (br d, J = 13.4 Hz, 1H), 6.69 (br dd, J = 7.2, 3.2 Hz, 1H), 4.68 (br s, 1H), 4.49 (br s, 1H), 4.47 (br s, 1H), 4.27 (br s, 1H), 4.01-3.86 (m, 1H), 3.12-2.96 (m, 1H), 2.89 (br dd, J = 5.8, 2.4 Hz, 3H), 2.30- 2.11 (m, 3H), 2.08-2.01 (m, 3H), 2.01-1.92 (m, 2H), 1.91- 1.60 (m, 4H), 1.46-1.31 (m, 1H) |
| 713 | 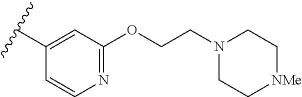 [SMILES: *c1ccnc(OCCN2CCN(C)CC2)c1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-2-(2-(4- methylpiperazin-1- yl)ethoxy)isonicotinamide | 589 | B: 0.99 | (500 MHz, DMSO-d₆) δ 8.60 (br d, J = 7.8 Hz, 1H), 8.29 (br d, J = 5.1 Hz, 1H), 8.01 (br s, 1H), 7.39 (br d, J = 5.1 Hz, 1H), 7.22 (s, 1H), 6.88-6.77 (m, 2H), 4.71 (br s, 1H), 4.57-4.45 (m, 3H), 4.29 (br s, 1H), 4.25-3.93 (m, |
| | | | | | 4H), 3.71-3.59 (m, 1H), 3.53- |
| | | | | | 3.28 (m, 2H), 3.22 (br d, J = |
| | | | | | 10.4 Hz, 1H), 3.18-3.09 (m, 1H), |
| | | | | | 3.05 (br d, J = 9.6 Hz, 1H), 2.81 |
| | | | | | (br s, 3H), 2.76-2.60 (m, 3H), |
| | | | | | 2.05, 2.01 (s, 3H), 1.95-1.82 |
| | | | | | (m, 3H), 1.53 (br s, 1H) |
| 714 | 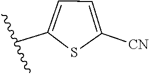 [SMILES: *c1ccc(C#N)s1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-5- | 477 | B: 1.17 | (500 MHz, DMSO-d₆) δ 8.77 (br d, J = 7.9 Hz, 1H), 8.02-7.96 (m, 1H), 7.92 (br d, J = 2.7 Hz, 1H), 7.87 (d, J = 1.5 Hz, 1H), 6.77 (br d, J = 11.9 Hz, 1H), 6.72 (d, |
| | | cyanothiophone-2- | | | J = 15.0 Hz, 1H), 4.69 (br s, 1H), |
| | | carboxamide | | | 4.49 (br s, 1H), 4.47 (br s, 1H), |
| | | | | | 4.27 (br s, 1H), 4.14 (br d, J = 5.2 |
| | | | | | Hz, 1H), 3.24-2.93 (m, 2H), 2.69 |
| | | | | | (br d, J = 0.9 Hz, 2H), 2.05, |
| | | | | | 2.01 (m, 3H), 1.96-1.76 (m, 3H), |
| | | | | | 1.52 (td, J = 7.7, 3.2 Hz, 1H) |
| 715 | 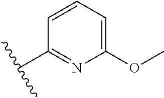 [SMILES: *c1cccc(OC)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6- methoxypicolinamide | 477 | B: 1.36 | (500 MHz, DMSO-d₆) δ 8.34 (br d, J = 6.4 Hz, 1H), 7.89-7.84 (m, 2H), 7.62 (d, J = 7.3 Hz, 1H), 7.02 (br d, J = 8.5 Hz, 1H), 6.76 (br d, J = 12.8 Hz, 1H), 6.71 (d, J = 4.6 Hz, 1H), 4.68 (br s, 1H), 4.48 (br s, 1H), 4.46 (br s, 1H), |
| | | | | | 4.26 (br s, 1H), 4.23-4.13 (m, |
| | | | | | 1H), 3.95 (s, 3H), 3.19-3.11 (m, |
| | | | | | 1H), 3.04-2.92 (m, 1H), 2.90- |
| | | | | | 2.69 (m, 2H), 2.05 and 2.01 (s, |
| | | | | | 3H), 1.94-1.77 (m, 3H), 1.72- |
| | | | | | 1.57 (m, 1H) |
| 716 | 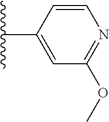 [SMILES: *c1ccnc(OC)c1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2- methoxyisonicotinamide | 477 | B: 1.13 | (500 MHz, DMSO-d₆) δ 8.61 (br d, J = 7.6 Hz, 1H), 8.27 (br d, J = 5.2 Hz, 1H), 7.86 (s, 1H), 7.32 (br d, J = 5.2 Hz, 1H), 7.16 (s, 1H), 6.77 (br d, J = 12.8 Hz, 1H), 6.70 (d, J = 9.2 Hz, 1H), 4.69 (br s, 1H), 4.49 (br s, 1H), 4.46 (br d, J = 0.6 Hz, 1H), 4.27 (br s, 1H), |
| | | | | | 4.16 (br s, 1H), 3.89 (s, 3H), 3.16 |
| | | | | | (br dd, J = 8.7, 1.7 Hz, 1H), |
| | | | | | 3.05-2.92 (m, 1H), 2.78-2.59 (m, |
| | | | | | 2H), 2.05, 2.01 (m, 3H), 1.96- |
| | | | | | 1.72 (m, 3H), 1.53 (br dd, |
| | | | | | J = 11.7, 6.9 Hz, 1H) |
| 717 | 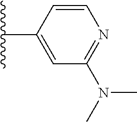 [SMILES: *c1ccnc(N(C)C)c1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-2- (dimethylamino) isonicotinamide | 490 | B: 0.99 | (500 MHz, DMSO-d₆) δ 8.48 (br d, J = 7.3 Hz, 1H), 8.14 (br d, J = 4.3 Hz, 1H), 7.86 (br d, J = 1.2 Hz, 1H), 6.92 (s, 1H), 6.88 (br d, J = 4.9 Hz, 1H), 6.77 (br d, J = 12.8 Hz, 1H), 6.70 (d, J = 6.7 Hz, 1H), 4.68 (br s, 1H), 4.48 (br s, 1H), 4.46 (br s, 1H), 4.27 (br s, |
| | | | | | 1H), 4.20-4.07 (m, 1H), 3.21- |
| | | | | | 3.13 (m, 1H), 3.04 (s, 6H), 3.01- |
| | | | | | 2.93 (m, 1H), 2.75-2.60 (m, 2H), |
| | | | | | 2.05, 2.01 (s, 3H), 1.95-1.73 |
| | | | | | (m, 3H), 1.59-1.43 (m, 1H) |
| 718 | 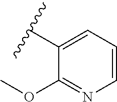 [SMILES: *c1cccnc1OC] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2- methoxynicotinamide | 477 | B: 1.21 | (500 MHz, DMSO-d₆) δ 8.30 (br s, 2H), 8.08 (br d, J = 6.7 Hz, 1H), 7.96 (s, 1H), 7.13 (br d, J = 4.3 Hz, 1H), 6.88-6.76 (m, 2H), 4.70 (br s, 1H), 4.51 (br s, 1H), 4.48 (br d, J = 1.2 Hz, 1H), 4.28 (br s, 1H), 4.25-4.15 (m, 1H), 3.97 |
| | | | | | (s, 3H), 3.20 (br d, J = 13.1 |
| | | | | | Hz, 1H), 2.95 (br s, 1H), 2.90- |
| | | | | | 2.73 (m, 2H), 2.06, 2.02 (s, 3H), |
| | | | | | 1.94-1.74 (m, 3H), 1.67-1.49 (m, 1H) |
| 719 | 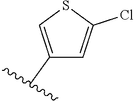 [SMILES: *c1csc(Cl)c1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-5- chlorothiophene-3- carboxamide | 486 | B: 1.32 | (500 MHz, DMSO-d₆) δ 8.27 (br d, J = 7.6 Hz, 1H), 8.00 (s, 1H), 7.95 (s, 1H), 7.49 (s, 1H), 6.84- 6.75 (m, 2H), 4.69 (br s, 1H), 4.50 (br s, 1H), 4.47 (br s, 1H), 4.27 (br s, 1H), 4.19-4.06 (m, 1H), 3.22-3.13 (m, 1H), 3.07- |
| | | | | | 2.98 (m, 1H), 2.75-2.60 (m, |
| | | | | | 2H), 2.10-1.99 (m, 3H), 1.93- |
| | | | | | 1.78 (m, 3H), 1.54-1.44 (m, |
| | | | | | 1H) |
| 720 | 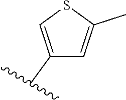 [SMILES: *c1csc(C)c1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-5- methylthiophene-3- carboxamide | 466 | B: 1.27 | (500 MHz, DMSO-d₆) δ 8.09 (br d, J = 7.6 Hz, 1H), 7.86 (s, 2H), 7.19 (s, 1H), 6.77 (br d, J = 12.2 Hz, 1H), 6.70 (d, J = 11.3 Hz, 1H), 4.69 (br s, 1H), 4.49 (br s, 1H), 4.47 (br s, 1H), 4.27 (br s, 1H), 4.17-4.05 (m, 1H), 3.20-3.11 |
| | | | | | (m, 1H), 3.00 (br dd, J = 4.4, |
| | | | | | 3.2 Hz, 1H), 2.73-2.59 (m, 2H), |
| | | | | | 2.44 (s, 3H), 2.05, 2.01 (s, 3H), |
| | | | | | 1.95-1.74 (m, 3H), 1.54-1.43 (m, 1H) |
| 721 | 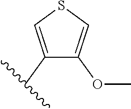 [SMILES: *c1cscc1OC] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- methoxythiophene-3- carboxamide | 482 | B: 1.25 | (500 MHz, DMSO-d₆) δ 8.01 (d, J = 3.4 Hz, 1H), 7.97 (s, 1H), 7.80-7.70 (m, 1H), 6.86-6.75 (m, 3H), 4.69 (br s, 1H), 4.51 (br s, 1H), 4.48 (br s, 1H), 4.28 (br d, J = 0.6 Hz, 1H), 4.24-4.14 (m, 1H), 3.88 (s, 3H), 3.19 (br d, J = |
| | | | | | 12.8 Hz, 1H), 2.98-2.90 (m, |
| | | | | | 1H), 2.87-2.72 (m, 2H), 2.05, |
| | | | | | 2.01 (m, 3H), 1.90-1.78 (m, 3H), |
| | | | | | 1.61-1.45 (m, 1H) |
| 722 | 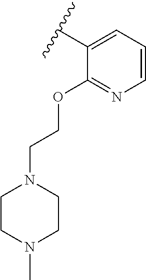 [SMILES: *c1cccnc1OCCN1CCN(C)CC1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2-(2-(4- methylpiperazin-1- yl)ethoxy)nicotinamide | 589 | B: 1.00 | (500 MHz, DMSO-d₆) δ 8.30 (br d, J = 4.0 Hz, 2H), 8.18 (dd, J = 7.5, 1.7 Hz, 1H), 7.87 (s, 1H), 7.14 (dd, J = 6.9, 5.3 Hz, 1H), 6.78 (br d, J = 13.4 Hz, 1H), 6.70 (br d, J = 10.1 Hz, 1H), 4.69 (br s, 1H), 4.48 (br d, J = 9.2 Hz, 4H), 4.27 (br s, 1H), 4.25-4.18 (m, 1H), 3.49-3.02 (m, 6H), 3.02- 2.90 (m, 1H), 2.89-2.75 (m, 1H), 2.75-2.61 (m, 2H), 2.44- 2.30 (m, 2H), 2.26-2.13 (m, 2H), 2.10-1.99 (m, 6H), 1.97- 1.78 (m, 3H), 1.62-1.48 (m, 1H) |
| 723 | 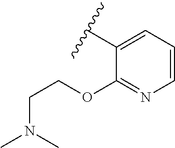 [SMILES: *c1cccnc1OCCN(C)C] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2-(2- (dimethylamino)ethoxy) nicotinamide | 534 | B: 0.98 | (500 MHz, DMSO-d₆) δ 8.49 (br d, J = 6.4 Hz, 1H), 8.29 (br d, J = 4.6 Hz, 1H), 8.18 (br d, J = 7.3 Hz, 1H), 7.85 (s, 1H), 7.18- 7.11 (m, 1H), 6.76 (br d, J = 12.2 Hz, 1H), 6.70 (br d, J = 5.2 Hz, 1H), 4.68 (br s, 1H), 4.52-4.42 (m, 4H), 4.29-4.24 (m, 1H), 4.20 (br s, 1H), 3.20-2.67 (m, 4H), 2.61 (br s, 2H), 2.14 (br s, 6H), 2.05, 2.01 (s, 3H), 1.96- 1.84 (m, 2H), 1.79 (br d, J = 9.5 Hz, 1H), 1.59-1.43 (m, 1H) |
| 724 | 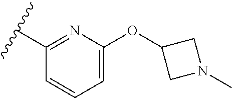 [SMILES: *c1cccc(OC2CN(C)C2)n1] | (R)-2-(1-(7-(1-acetyl-2,5- dihydro-1H-pyrrol-3-yl)- 4-aminopyrrolo[2,1- f][1,2,4]triazin-5- yl)piperidin-3-yl)-1,1,3,3- tetramethylguanidine | 532 | B: 1.01 | (500 MHz, DMSO-d₆) δ 8.08 (br dd, J = 5.9, 1.3 Hz, 1H), 7.98 (br t, J = 7.7 Hz, 1H), 7.91 (s, 1H), 7.70 (br d, J = 6.4 Hz, 1H), 7.16 (br t, J = 7.3 Hz. 1H), 6.79 (br d. J = 6.9 Hz, 1H), 6.73 (br d, J = 9.7 Hz, 1H), 5.72-5.46 (m, 1H), 4.70 (br s, 1H), 4.49 (br d, J = 9.7 Hz, |
| | | | | | 2H), 4.44-4.32 (m, 1H), 4.27 |
| | | | | | (br s, 1H), 4.25-4.18 (m, 1H), |
| | | | | | 4.17-4.09 (m, 1H), 3.22-3.15 |
| | | | | | (m, 1H), 3.11 (s, 3H), 3.05 (br d, |
| | | | | | J = 7.4 Hz, 1H), 2.91 (br d, J = 16.0 |
| | | | | | Hz, 3H), 2.85-2.72 (m, 1H), |
| | | | | | 2.05, 2.01 (s. 3H), 1.94-1.81 |
| | | | | | (m, 3H), 1.69-1.57 (m, 1H) |
| 725 | 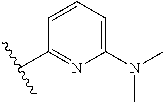 [SMILES: *c1cccc(N(C)C)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6- (dimethylamino) picolinamide | 490 | B: 1.35 | (500 MHz, DMSO-d₆) δ 8.27 (br s, 1H), 7.86 (br d, J = 1.8 Hz, 1H), 7.65 (br t, J = 7.8 Hz, 1H), 7.22 (d, J = 7.3 Hz, 1H), 6.84 (br d, J = 8.5 Hz, 1H), 6.77 (br d, J = 13.1 Hz, 1H), 6.72 (br d, J = 9.2 Hz, 1H), 4.69 (br s, 1H), 4.49 (br s, |
| | | | | | 1H), 4.47 (br s, 1H), 4.26 (br s, |
| | | | | | 1H), 4.21-4.10 (m, 1H), 3.20- |
| | | | | | 3.13 (m, 1H), 3.07 (s, 6H), 3.00- |
| | | | | | 2.91 (m, 1H), 2.89-2.63 (m, |
| | | | | | 2H), 2.05 & 2.01 (s, 3H), 1.94- |
| | | | | | 1.77 (m, 3H), 1.71-1.54 (m, 1H) |
| 726 | 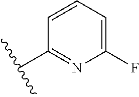 [SMILES: *c1cccc(F)n1] | :(R)-N-(1-(7-(1-acetyl- 2,5-dihydro-1H-pyrrol-3- yl)-4-aminopyrrolo[2,1- f][1,2,4]triazin-5- yl)piperidin-3-yl)-6- fluoropicolinamide | 465 | B: 1.21 | (500 MHz, DMSO-d₆) δ 8.53 (br dd, J = 8.1, 4.4 Hz, 1H), 8.19 (q, J = 7.9 Hz, 1H), 7.97 (br d, J = 7.4 Hz, 1H), 7.94 (s, 1H), 7.43 (br d, J = 8.0 Hz, 1H), 6.81 (br s, 1H), 6.77 (br d, J = 16.8 Hz, 1H), 4.70 (br s, 1H), 4.51 (br s, 1H), |
| | | | | | 4.48 (br s, 1H), 4.27 (br s, 1H), |
| | | | | | 4.24-4.17 (m, 1H), 3.17 (br d, J = |
| | | | | | 10.5 Hz, 1H), 3.04 (br d, J = 5.1 |
| | | | | | Hz, 1H), 2.80 (br t, J = 9.6 Hz, |
| | | | | | 1H), 2.70 (br d, J = 6.7 Hz, 1H), |
| | | | | | 2.03 (d, J = 19.3 Hz, 3H), 1.92- |
| | | | | | 1.82 (m, 3H), 1.67 (br d, J = 7.8 |
| | | | | | Hz, 1H) |
| 727 | 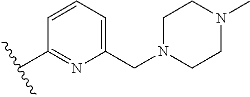 [SMILES: *c1cccc(CN2CCN(C)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-((4- methylpiperazin-1- yl)methyl)picolinamide | 559 | B: 0.91 | (500 MHz, METHANOL-d₄) δ 8.08-8.03 (m, 1H), 8.02-7.98 (m, 1H), 7.96 (d, J = 2.4 Hz, 1H), 7.71 (ddd, J = 7.6, 2.6, 1.1 Hz, 1H), 6.96 (dt, J = 4.2, 2.1 Hz, 1H), 6.90 (d, J = 9.0 Hz, 1H), 4.82- 4.78 (m, 1H), 4.69-4.65 (m, |
| | | | | | 1H), 4.61-4.58 (m, 1H), 4.47- |
| | | | | | 4.41 (m, 1H), 4.40-4.31 (m, |
| | | | | | 1H), 3.97-3.87 (m, 2H), 3.45- |
| | | | | | 3.36 (m, 4H), 3.30-3.19 (m, 3H), |
| | | | | | 3.03-2.78 (m, 8H), 2.17 (s, |
| | | | | | 3H), 2.11 (ddd, J = 4.8, 3.1, 1.7 Hz, |
| | | | | | 1H), 2.04-1.95 (m, 2H), 1.79- |
| | | | | | 1.67 (m, 1H) |
| 728 | 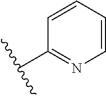 [SMILES: *c1ccccn1] | (R)-N-(1-(7-(1-acetyl-2,5- dihydro-1H-pyrrol-3-yl)- 4-aminopyrrolo[2,1- f][1,2,4]triazin-5- yl)piperidin-3- yl)picolinamide | 447 | B: 1.11 | (500 MHz, DMSO-d₆) δ 8.81- 8.55 (m, 2H), 8.14-7.94 (m, 3H), 7.63 (br d, J = 4.9 Hz, 1H), 6.87-6.77 (m, 2H), 4.70 (br s, 1H), 4.51 (br s, 1H), 4.48 (br s, 1H), 4.28 (br s, 1H), 4.26-4.19 |
| | | | | | (m, 1H), 3.25-3.15 (m, 1H), 3.09- |
| | | | | | 2.96 (m, 1H), 2.88-2.69 (m, |
| | | | | | 2H), 2.05 and 2.01(s, 3H), 1.93- |
| | | | | | 1.79 (m, 3H), 1.74-1.60 (m, |
| | | | | | 1H) |
| 729 | 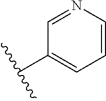 [SMILES: *c1cccnc1] | (R)-N-(1-(7-(1-acetyl-2,5- dihydro-1H-pyrrol-3-yl)- 4-aminopyrrolo[2,1- f][1,2,4]triazin-5~ yl)piperidin-3- yl)nicotinamide | 447 | B: 0.99 | (500 MHz, DMSO-d₆) δ 9.13 (br s, 1H), 8.85 (br s, 1H), 8.64 (br d, J = 7.9 Hz, 1H), 8.25 (br d, J = 7.9 Hz, 1H), 8.02 (br s, 1H), 7.60 (br d, J = 0.9 Hz, 1H), 6.89-6.77 (m, 2H), 4.71 (br s, 1H), 4.52 (br s, 1H), 4.49 (br s, 1H), 4.28 |
| | | | | | (br s, 1H), 4.27-4.18 (m, 1H), |
| | | | | | 3.25 (br d, J = 10.7 Hz, 1H), 3.04 |
| | | | | | (br d, J = 9.2 Hz, 1H), 2.73 (br d, |
| | | | | | J = 9.2 Hz, 2H), 2.05 and 2.01 |
| | | | | | (s, 3H), 1,98-1.79 (m, 3H), 1.55 |
| | | | | | (br s, 1H) |
| 730 | 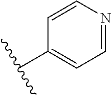 [SMILES: *c1ccncc1] | (R)-N-(1-(7-(1-acetyl-2,5- dihydro-1H-pyrrol-3-yl)- 4-aminopyrrolo[2,1- f][1,2,4]triazin-5- yl)piperidin-3- yl)isonicotinamide | 447 | B: 0.99 | (500 MHz, DMSO-d₆) δ 8.76 (br s, 2H), 8.72 (br d, J = 7.3 Hz, 1H), 7.99 (s, 1H), 7.80 (br d, J = 4.9 Hz, 2H), 6.87-6.77 (m, 2H), 4.71 (br s, 1H), 4.52 (br d, J = 1.2 Hz, 1H), 4.49 (br s, 1H), 4.28 (br s, 1H), 4.26-4.18 (m, 1H), |
| | | | | | 3.23 (br d, J = 10.7 Hz, 1H), 3.04 |
| | | | | | (br dd, J = 7.2, 2.9 Hz, 1H), 2.81- |
| | | | | | 2.66 (m, 2H), 2.06 and 2.02 (s, |
| | | | | | 3H), 1.96-1.82 (m, 3H), 1.62-1.51 |
| | | | | | (m, 1H) |
| 731 | 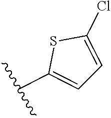 [SMILES: *c1ccc(Cl)s1] | (R)-N-(1-(7-(1-acctyl-2,5- dihydro-1H-pyrrol-3-yl)- 4-aminopyrrolo[2,1- f][1,2,4]triazin-5- yl)piperidin-3-yl)-5- chlorothiophene-2- carboxamide | 486 | B: 1.39 | (500 MHz, DMSO-d₆) δ 8.48 (br d, J = 7.3 Hz, 1H), 7.97 (s, 1H), 7.75-7.68 (m, 1H), 7.21-7.14 (m, 1H), 6.85-6.74 (m, 2H), 4,70 (br d, J = 1.2 Hz, 1H), 4.51 (br d, J = 1.8 Hz, 1H), 4.48 (br s, 1H), 4.28 (br s, 1H), 4.20-4.08 (m, 1H), 3.24-3.15 (m, 1H), |
| | | | | | 3.08-3.01 (m, 1H), 2.77-2.61 (m, |
| | | | | | 2H), 2.05 and 2.02 (s, 3H), |
| | | | | | 1.96-1.78 (m, 3H), 1.58-1.42 |
| | | | | | (m, 1H) |
| 732 | 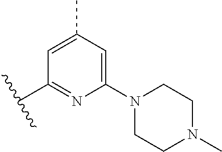 | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- methyl-6-(4- methylpiperazin-1- yl)picolinamide | 559 | B: 1.05 | (500 MHz, DMSO-d₆) δ 8.21 (br s, 1H), 7.87 (br d, J = 1.5 Hz, 1H), 7.16 (s, 1H), 6.86 (s, 1H), 6.77 (br d, J = 12.5 Hz, 1H), 6.71 (br d, J = 10.1 Hz, 1H), 4.69 (br s, 1H), 4.49 (br s, 1H), 4.47 (br s, 1H), 4.27 (br s, 1H), 4.21-4.10 (m, 1H), 3.55-3.40 (m, 4H), 3.20-3.10 (m, 1H), 3.03-2.92 (m, 1H), 2.88-2.67 (m, 2H), 2.41 (br s, 4H), 2.28 (s, 3H), 2.22 (s, 3H), 2.05 & 2.01 (s, 3H), 1.93-1.76 (m, 3H), 1.69-1.57 (m, 1H) |
| 733 | 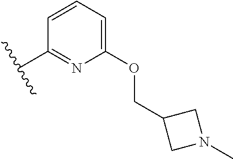 [SMILES: *c1cccc(OCC2CN(C)C2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-((1- methylazetidin-3- yl)methoxy)picolinamide | 546 | B: 1.01 | (500 MHz, DMSO-d₆) δ 8.34 (br d, J = 8.2 Hz, 1H), 7.90-7.81 (m, 2H), 7.62 (d, J = 7.3 Hz, 1H), .02 (br d, J = 8.2 Hz, 1H), 6.76 (br d, J = 12.2 Hz, 1H), 6.71 (br d, J = 3.4 Hz, 1H), 4.67 (br s, 1H), 4.54-4.41 (m, 4H), 4.26 (br s, 1H), 4.23-4.14 (m, 1H), 3.39 (br d, J = 5.2 Hz, 1H), 3.16 (br d, J = 13.7 Hz, 3H), 2.98-2.73 (m, 3H), 2.28 (br d, J = 2.7 Hz, 3H), 2.05 & 2.01 (s, 3H), 1.91-1.77 (m, 4H), 1.74-1.61 (m, 1H) |
| 734 | 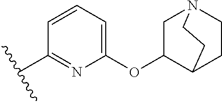 [SMILES: *c1cccc(OC2CN3CCC2CC3)n1] | N-((R)-1-(7-(1-acetyl-2,5- dihydro-1H-pyrrol-3-yl)- 4-aminopyrrolo[2,1- f][1,2,4]triazin-5- yl)piperidin-3-yl)-6- (quinuclidin-3- yloxy)picolinamide | 572 | B: 1.08 | (500 MHz, DMSO-d₆) δ 8.23- 8.12 (m, 1H), 8.00-7.89 (m, 2H). 7.68 (d, J = 7.3 Hz, 1H), 7.09 (br d, J = 8.2 Hz, 1H), 6.84- 6.72 (m, 2H), 5.47 (br d, J = 4.3 Hz, 1H), 4.70 (br s, 1H), 4.50 (br s, 1H), 4.48 (br s, 1H), 4.28 (br s, 1H), 4.25-4.17 (m, 1H), 3.84 |
| | | | | | (br dd, J = 12.8, 8.5 Hz, 1H), 3.36- |
| | | | | | 3.14 (m, 4H), 3.10-2.99 (m, |
| | | | | | 1H), 2.91-2.70 (m, 2H), 2.45 (br |
| | | | | | d, J = 7.9 Hz, 1H), 2.21-2.10 |
| | | | | | (m, 1H), 2.05 & 2.01 (s, 3H), 1.99- |
| | | | | | 1.76 (m, 6H), 1.74-1.59 (m, |
| | | | | | 1H) |
| 735 | 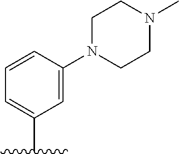 [SMILES: *c1cccc(N2CCN(C)CC2)c1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-3-(4- methylpiperazin-1- yl)benzamide | 544 | B: 0.99 | (500 MHz, DMSO-d₆) δ 8.25 (br d, J = 7.1 Hz, 1H), 7.87 (s, 1H), 7.33 (br s, 1H), 7.30-7.21 (m, 2H), 7.07 (br d, J = 8.4 Hz, 1H), 6.77 (br d, J = 12.4 Hz, 1H), 6.73- 6.67 (m, 1H), 4.69 (br s, 1H), 4.49 (br s, 1H), 4.47 (br s, 1H), 4.26 (br s, 1H), 4.21-4.09 (m, 1H), 3.47 (br d, J = 5.6 Hz, 4H), 3.17 (br s, 2H), 3.07-2.95 (m, |
| | | | | | 1H), 2.71-2.60 (m, 1H), 2.49-2.43 |
| | | | | | (m, 4H), 2.23 (s, 3H), 2.05 |
| | | | | | & 2.01 (s, 3H), 1.95-1.73 (m, |
| | | | | | 3H), 1.61-1.46 (m, 1H) |
| 736 | 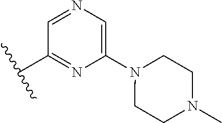 [SMILES: *c1cncc(N2CCN(C)CC2)n1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-6-(4- methylpiperazin-1- yl)pyrazine-2- carboxamide | 546 | B: 1.19 | (500 MHz, DMSO-d₆) δ 8.48 (s, 1H), 8.34 (s, 1H), 8.31 (br d, J = 8.0 Hz, 1H), 7.86 (d, J = 1.7 Hz, 1H), 6.76 (br d, J = 12.0 Hz, 1H), 6.70 (d, J = 9.2 Hz, 1H), 4.68 (br s, 1H), 4.48 (br s, 1H), 4.46 (br s, 1H), 4.26 (br s, 1H), 4.22- 4.13 (m, 1H), 3.72-3.56 (m, 4H), 3.16-3.07 (m, 1H), 3.03- |
| | | | | | 2.94 (m, 1H), 2.88-2.78 (m, |
| | | | | | 1H), 2.77-2.66 (m, 1H), 2.45 (br |
| | | | | | t, J = 4.2 Hz, 4H), 2.24 (s, 3H), |
| | | | | | 2.05 & 2.01 (s, 3H), 1.89-1.76 |
| | | | | | (m, 3H), 1.70-1.59 (m, 1H) |
| 737 | 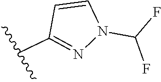 [SMILES: *c1ccn(C(F)F)n1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-1- (difluoromethyl)-1H- pyrazole-3-carboxamide | 486 | B: 1.17 | (500 MHz, DMSO-d₆) δ 8.32 (br d, J = 1.6 Hz, 2H), 8.02-7.72 (m, 2H), 6.88 (d, J = 2.7 Hz, 1H), 6.77 (br d, J = 13.2 Hz, 1H), 6.70 (d, J = 13.4 Hz, 1H), 4.69 (br s, 1H), 4.49 (br s, 1H), 4.46 (br d, J = 0.9 Hz, 1H), 4.26 (br s, 1H), |
| | | | | | 4.22-4.10 (m, 1H), 3.19-3.10 |
| | | | | | (m, 1H), 3.04-2.93 (m, 1H), 2.83- |
| | | | | | 2.59 (m, 2H), 2.05 & 2.01 (s, |
| | | | | | 3H), 1.93-1.73 (m, 3H), 1.66-1.52 |
| | | | | | (m, 1H) |
| 738 | 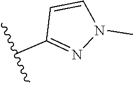 [SMILES: *c1ccn(C)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-1- methyl-1H-pyrazole-3- carboxamide | 450 | B: 1.28 | (500 MHz, DMSO-d₆) δ 7.93 (br dd, J = 7.8, 3.9 Hz, 1H), 7.85 (d, J = 1.7 Hz, 1H), 7.73 (s, 1H), 6.76 (br d, J = 13.8 Hz, 1H), 6.69 (d, J = 8.1 Hz, 1H), 6.61 (d, J = 1.4 Hz, 1H), 4.68 (br s, 1H), 4.48 (br s, 1H), 4.46 (br s, 1H), 4.30-4.22 |
| | | | | | (m, 1H), 4.18-4.05 (m, 1H), |
| | | | | | 3.88 (s, 3H), 3.16-3.06 (m, 1H), |
| | | | | | 3.02-2.89 (m, 1H), 2.81-2.59 |
| | | | | | (m, 2H), 2.09-1.97 (m, 3H), 1.90- |
| | | | | | 1.71 (m, 3H), 1.62-1.47 (m, |
| | | | | | 1H) |
| 739 | 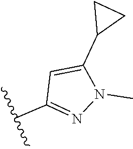 [SMILES: *c1cc(C2CC2)n(C)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-5- cyclopropyl-1-methyl-1H- pyrazole-3-carboxamide | 490 | B: 1.40 | (500 MHz, DMSO-d₆) δ 8.25 (br d, J = 7.7 Hz, 1H), 7.85 (d, J = 1.3 Hz, 1H), 6.76 (br d, J = 12.8 Hz, 1H), 6.69 (d, J = 7.7 Hz. 1H), 6.52 (d, J = 1.1 Hz, 1H), 4.68 (br s, 1H), 4.48 (br s, 1H), 4.46 (br s, 1H), 4.26 (br s, 1H), 4.08 (br dd, J = 6.5, 2.2 Hz, 1H), 3.90 (s, 3H), 3.17-3.08 (m, 1H), 3.01- 2.92 (m, 1H), 2.73-2.58 (m, |
| | | | | | 2H), 2.08-1.98 (m, 3H), 1,92- |
| | | | | | 1.80 (m, 3H), 1.79-1.68 (m, |
| | | | | | 1H), 1.54-1.40 (m, 1H), 0.90- |
| | | | | | 0.81 (m, 2H), 0.65-0.55 (m, |
| | | | | | 2H) |
| 740 | 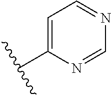 [SMILES: *c1ccncn1] | (R)-N-(1-(7-(1-acetyl-2,5- dihydro-1H-pyrrol-3-yl)- 4-aminopyrrolo[2,1- f][1,2,4]triazin-5- yl)piperidin-3- yl)pyrimidine-4- carboxamide | 448 | B: 1.20 | (500 MHz, DMSO-d₆) δ 9.35 (s, 1H), 9.07 (d, J = 5.2 Hz, 1H), 8.90 (br dd, J = 7.9, 4.9 Hz, 1H), 8.02 (br d, J = 4.9 Hz, 1H), 7.87 (d, J = 2.1 Hz, 1H), 6.77 (br d, J = 13.7 Hz, 1H), 6.72 (br d, J = 11.9 Hz, 1H), 4.69 (br s, 1H), 4.49 (br s, |
| | | | | | 1H), 4.47 (br s, 1H), 4.27 (br s, |
| | | | | | 1H), 4.22 (br dd, J = 5.5, 2.4 Hz, |
| | | | | | 1H), 3.14 (br d, J = 10.4 Hz, |
| | | | | | 1H), 3.05-2.92 (m, 1H), 2.89- |
| | | | | | 2.65 (m, 2H), 2.09-1.98 (m, |
| | | | | | 3H), 1.93-1.77 (m, 3H), |
| | | | | | 1.72-1.63 (m, 1H) |
| 741 | 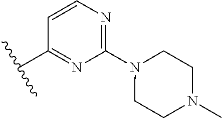 [SMILES: *c1ccnc(N2CCN(C)CC2)n1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-2-(4- methylpiperazin-1- yl)pyrimidine-4- carboxamide | 546 | B: 1.08 | (500 MHz, DMSO-d₆) δ 8.66 (d, J = 4.9 Hz, 1H), 8.55 (br d, J = 8.9 Hz, 1H), 7.95 (s, 1H), 7.24 (d, J = 4.6 Hz, 1H), 6.80 (br d, J = 8.9 Hz, 1H), 6.76 (d, J = 12.2 Hz, 1H), 4.70 (br s, 1H), 4.50 (br s, 1H), 4.48 (br s, 1H), 4.28 (br s, 1H), 4.25-4.17 (m, 1H), 3.90 (br s, 4H), 3.22-3.10 (m, 2H), 3.05 |
| | | | | | (br d, J = 9.8 Hz, 1H), 2.86 (s, |
| | | | | | 4H), 2.76-2.66 (m, 1H), 2.55 (s, |
| | | | | | 3H), 2.05 & 2.01 (s, 3H), 1.94- |
| | | | | | 1.79 (m, 3H), 1.72-1.60 (m, 1H) |
| 742 | 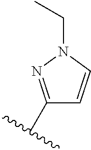 [SMILES: *c1ccn(CC)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-1-ethyl- 1H-pyrazole-3- carboxamide | 464 | B: 1.11 | (500 MHz, DMSO-d₆) δ 7.95- 7.84 (m, 3H), 7.81 (s, 1H), 6.77 (br d, J = 13.7 Hz, 1H), 6.70 (d, J = 14.0 Hz, 1H), 6.62 (d, J = 1.2 Hz, 1H), 4.69 (br s, 1H), 4.50 (br s, 1H), 4.47 (br s, 1H), 4.27 (br s, 1H), 4.19 (q, J = 7.4 Hz, 2H), 4.16-4.08 (m, 1H), 3.19-3.11 (m, 1H), 3.04-2.94 (m, 1H), 2.80- 2.60 (m, 2H), 2.08-1.99 (m, 3H), 1.91-1.74 (m, 3H), 1.65- 1.50 (m, 1H), 1.40 (t, J = 7.2 Hz, 3H) |
| 743 | 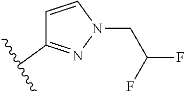 [SMILES: *c1ccn(CC(F)F)n1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-1-(2,2- difluoroethyl)-1H- pyrazole-3-carboxamide | 500 | B: 1.17 | (500 MHz, DMSO-d₆) δ 8.03 (br dd, J = 8.2, 3.1 Hz, 1H), 7.98 (s. 1H), 7.86 (d, J = 1.2 Hz, 1H), 6.83 (s, 1H), 6.80-6.76 (m, 1H), 6.71 (d, J = 2.4 Hz, 1H), 6.54- 6.27 (m, 1H), 4.70 (td, J = 15.2, 3.2 Hz, 3H), 4.51 (br s, 1H), 4.48 |
| | | | | | (br s, 1H), 4.28 (br s, 1H), 4.24- |
| | | | | | 4.14 (m, 1H), 3.18 (br d, J = |
| | | | | | 8.2 Hz, 1H), 3.08-2.98 (m, 1H), |
| | | | | | 2.78-2.63 (m, 2H), 2.05 & 2.01 |
| | | | | | (s, 3H), 1.92-1.80 (m, 3H), |
| | | | | | 1.63-1.52 (m, 1H) |
| 744 | 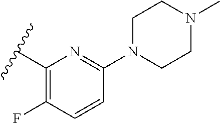 [SMILES: *c1nc(N2CCN(C)CC2)ccc1F] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-3- fluoro-6-(4- methylpiperazin-1- yl)picolinamide | 563 | B: 1.01 | (500 MHz, DMSO-d₆) δ 8.38- 8.26 (m, 1H), 7.99 (s, 1H), 7.68 (t, J = 9.6 Hz, 1H), 7.18 (dd, J = 9.2, 2.1 Hz, 1H), 6.86-6.75 (m, 2H), 4.70 (br s, 1H), 4.51 (br s, 1H), 4.48 (br s, 1H), 4.28 (br s, 1H), 4.24-4.14 (m, 1H), 3.23- 3.14 (m, 1H), 3.06-2.96 (m, 1H), 2.86 (s, 3H), 2.77 (br d, J = 7.9 Hz, 2H), 2.55 (s, 4H), 2.09- 1.98 (m, 3H), 1.95-1.78 (m, 3H), 1.58 (br s, 1H). |
| 745 | 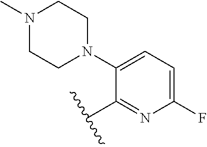 [SMILES: *c1nc(F)ccc1N1CCN(C)CC1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6- fluoro-3-(4- methylpiperazin-1- yl)picolinamide | 563 | B: 1.03 | (500 MHz, DMSO-d₆) δ 8.62 (br d, J = 6.4 Hz, 1H), 8.00 (d, J = 1.2 Hz, 1H), 7.88 (br t, J = 7.8 Hz, 1H), 7.32-7.25 (m, 1H), 6.86-6.76 (m, 2H), 4.71 (br s, 1H), 4.52 (br s, 1H), 4.49 (br s, 1H), 4.28 (br s, 1H), 4.19-4.19 (m, 1H), 4.24-4.18 (m, 1H), 3.37 (br d, J = 11.3 Hz, 1H), 3.24- 3.16 (m, 1H), 3.13-2.89 (m, 2H), 2.78 (br s, 4H), 2.55 (s, 3H), 2.06 & 2.02 (s, 3H), 1.96- 1.73 (m, 3H), 1.62-1.48 (m, 1H). |
| 746 | 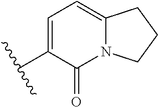 [SMILES: *c1ccc2n(c1=O)CCC2] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-5-oxo- 1,2,3,5- tetrahydroindolizine-6- carboxamide | 503 | B: 1.15 | (500 MHz, DMSO-d₆) δ 10.31 (br s, 1H), 8.28 (d, J = 7.6 Hz, 1H), 7.98 (s, 1H), 6.94-6.85 (m, 1H), 6.81 (br d, J = 15.0 Hz, 1H), 6.51 (d, J = 7.3 Hz, 1H), 4.70 (br s, 1H), 4.51 (br s, 1H), 4.48 (br d, J = 1.5 Hz, 1H), 4.32-4.21 (m, 2H), 4.21-4.10 (m, 2H), 3.25- |
| | | | | | 3.07 (m, 2H), 2.98-2.78 (m, 2H), |
| | | | | | 2.18 (quin, J = 7.5 Hz, 2H), |
| | | | | | 2.11-1.98 (m, 3H), 1.91-1.72 |
| | | | | | (m, 3H), 1.67-1.49 (m, 1H) |
| 747 | 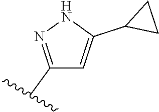 [SMILES: *c1cc(C2CC2)[nH]n1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-3- cyclopropyl-1H-pyrazole- 5-carboxamide | 476 | B: 1.21 | (500 MHz, DMSO-d₆) δ 7.87 (br d, J = 2.4 Hz, 1H), 6.77 (br d, J = 13.7 Hz. 1H), 6.69 (d, J = 14.0 Hz, 1H), 6.27 (br s, 1H), 4.69 (br s, 1H), 4.49 (br s, 1H), 4.47 (br s, 1H), 4.27 (br d, J = 2.1 Hz, 1H), 4.17-4.05 (m, 1H), 3.21- 3.07 (m, 1H), 3.03-2.91 (m, |
| | | | | | 1H), 2.78-2.57 (m, 2H), 2.05 & |
| | | | | | 2.01 (s, 3H), 1.97-1.71 (m, |
| | | | | | 4H), 1.64-1,44 (m, 1H), 0.94 (br |
| | | | | | dd, J = 3.1, 2.1 Hz, 2H), 0.69 |
| | | | | | (br d, J = 1.2 Hz, 2H) |
| 748 | 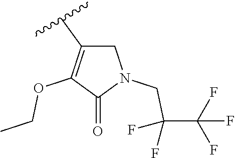 [SMILES: *C1=C(OCC)C(=O)N(CC(F)(F)C(F)(F)F)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)~4- ethoxy-5-oxo-1- (2,2,3,3,3- pentafluoropropyl)-2,5- dihydro-1H-pyrrole-3- carboxamide | 627 | B: 1.55 | (500 MHz, DMSO-d₆) δ 7.75 (br s, 1H), 6.77 (br d, J = 13.4 Hz, 1H), 6.73 (br d, J = 10.1 Hz, 1H), 4.69 (br s, 1H), 4.61-4.52 (m, 2H), 4.49 (br s, 1H), 4.47 (br s, 1H), 4.39-4.22 (m, 3H), 4.15 (s, 2H), 3.50-3.38 (m, 1H), 3.13- 3.00 (m, 1H), 2.98-2.68 (m, 2H), 2.06 & 2.01 (s, 3H), 1.81 (br s, 3H), 1.55 (br dd, J = 12.4, 2.3 Hz, 1H), 1.25 (br t, J = 6.9 Hz, 3H) |
| 749 | 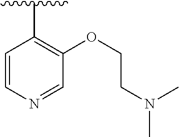 [SMILES: *c1ccncc1OCCN(C)C] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-3-(2- (dimethylamino)ethoxy) isonicotinamide | 534 | B: 0.90 | (500 MHz, DMSO-d₆) δ 8.58 (s, 1H), 8.52 (br d, J = 7.0 Hz, 1H). 8.32 (d, J = 4.9 Hz, 1H), 7.87 (s, 1H), 7.64 (d, J = 4.9 Hz, 1H), 6.78 (br d, J = 12.5 Hz, 1H), 6.71 (d, J = 15.6 Hz, 1H), 4.69 (br s, 1H), 4.49 (br s, 1H), 4.47 (br s, 1H), 4.33 (br t, J = 5.2 Hz, 2H), 4.27 (br s, 1H), 4.25-4.17 (m, 1H), |
| | | | | | 3.18 (s, 2H), 3.04-2.89 (m, 1H), |
| | | | | | 2.85-2.84 (m, 1H), 2.85-2.68 (m, |
| | | | | | 1H), 2.62 (br d, J = 3.4 Hz, |
| | | | | | 2H), 2.16 (br s, 6H), 2.06 & 2.02 |
| | | | | | (s, 3H), 1.92-1.75 (m, 3H), |
| | | | | | 1.57-1.42 (m, 1H) |
| 750 | 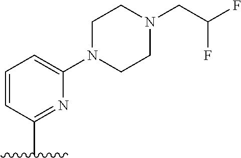 [SMILES: *c1cccc(N2CCN(CC(F)F)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-(4- (2,2- difluoroethyl)piperazin-1- yl)picolinamide | 595 | B: 1.08 | (500 MHz, DMSO-d₆) δ 8.21 (br d, J = 7.3 Hz, 1H), 7.87 (d, J = 2.1 Hz, 1H), 7.69 (t, J = 7.9 Hz, 1H), 7.30 (d, J = 7.3 Hz, 1H), 7.04 (br d, J = 8.5 Hz, 1H), 6.77 (br d, J = 12.2 Hz, 1H), 6.72 (d, J = 14.6 Hz, 1H), 6.19 (tt, J = 56.2, 4.0 Hz, 1H), 4.69 (br s, 1H), 4.50 (br s, 1H), 4.47 (br s, 1H), 4.27 (br s, 1H), 4.22-4.11 (m, 1H), 3.56 (br s, 4H), 3.21-3.11 |
| | | | | | (m, 1H), 3.06-2.92 (m, 1H), |
| | | | | | 2.80 (td, J = 15.7, 4.0 Hz, 4H), 2.64 |
| | | | | | (br t, J = 4.3 Hz, 4H), 2.05 & |
| | | | | | 2.01 (s, 3H), 1.95-1.86 (m, 1H), 1.83 |
| | | | | | (br d, J = 4.0 Hz, 2H), 1.73- |
| | | | | | 1.55 (m, 1H) |
| 751 | 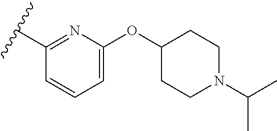 [SMILES: *c1cccc(OC2CCN(C(C)C)CC2)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-6-((1- isopropylpiperidin-4- yl)oxy)picolinamide | 588 | B: 1.11 | (500 MHz, DMSO-d₆) δ 7.92-7.84 (m, 2H), 7.60 (d, J = 7.0 Hz, 1H), 7.01 (br d, J = 8.9 Hz, 1H), 6.77 (br d, J = 14.6 Hz, 1H), 6.73 (d, J = 14.6 Hz, 1H), 5.18-4.96 (m, 1H), 4.69 (br s, 1H), 4.50 (br s, 1H), 4.47 (br s, 1H), 4.27 (br s, 1H), 4.23-4.13 (m, 1H), 3.18- 3.06 (m, 1H), 2.94 (br dd, J = |
| | | | | | 5.5, 2.7 Hz, 2H), 2.76-2.64 (m, |
| | | | | | 2H), 2.37-2.11 (m, 2H), 2.11- |
| | | | | | 1.96 (m, 5H), 1.92 (s, 3H), 1.88- |
| | | | | | 1.73 (m, 3H), 1.70-1.55 (m, 2H), |
| | | | | | 0.87 (br s, 6H) |
| 752 | 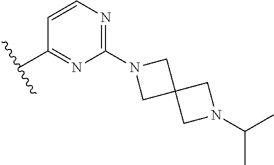 [SMILES: *c1ccnc(N2CC3(C2)CN(C(C)C)C3)n1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-2-(6- isopropyl-2,6- diazaspiro[3.3]heptan-2- yl)pyrimidine-4- carboxamide | 586 | B: 1.08 | (500 MHz, DMSO-d₆) δ 8.56 (d, J = 4.6 Hz, 1H), 8.35 (br s, 1H), 7.88 (d, J = 1.5 Hz, 1H), 7.14 (d, J = 4.9 Hz, 1H), 6.78 (br d, J = 13.4 Hz, 1H), 6.73 (br d, J = 15.9 Hz, 1H), 4.70 (br s, 1H), 4.50 (br s, 1H), 4.47 (br s, 1H), 4.31- 4.24 (m, 1H), 4.23-4.07 (m, 5H), 3.18-3.08 (m, 1H), 3.04- 2.89 (m, 2H), 2.88-2.72 (m, 1H), 2.40-2.23 (m, 1H), 2.10- |
| | | | | | 1.99 (m, 3H), 1.92-1.78 (m, |
| | | | | | 3H), 1.70-1.58 (m, 1H), 0.87 |
| | | | | | (br d, J = 5.8 Hz, 6H) |
| | | | | | (4 Hs were absent due |
| | | | | | to saturation.) |
| 753 | 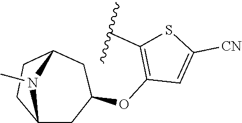 [SMILES: *c1sc(C#N)cc1O[C@@H]1C[C@H]2CC[C@@H](C1)N2C] | N-((R)-1-(7-(1-acetyl-2,5- dihydro-1H-pyrrol-3-yl)- 4-aminopyrrolo[2,1- f\|[1,2,4]triazin-5- yl)piperidin-3-yl)-5- cyano-3-(((1R,3s,5S)-8- methyl-8- azabicyclo[3.2.1]octan-3- yl)oxy)thiophene-2- | 616 | B: 1.31 | (500 MHz, DMSO-d₆) δ 8.28 (s, 1H), 7.96 (s, 1H), 7.94 (s, 1H), 7.47-7.35 (m, 1H), 6.83-6.73 (m, 2H), 5.01-4.85 (m, 1H), 4.69 (br s, 1H), 4.49 (br d, J = 5.8 Hz, 2H), 4.28 (br s, 1H), 4.24- 4.15 (m, 1H), 3.99 (br s, 2H), 3.17-3.12 (m, 1H), 3.07-3.00 (m, 1H), 2.85-2.76 (m, 2H), 2.70 |
| | | carboxamide | | | (br s, 3H), 2.44-2.37 (m, 2H), |
| | | | | | 2.26-2.08 (m, 6H), 2.04 (d, J = |
| | | | | | 19.3 Hz, 3H), 1.98-1.85 (m, |
| | | | | | 2H), 1.83-1.74 (m, 1H), 1.60-1.47 |
| | | | | | (m, 1H) |
| 754 | 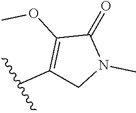 | (R)-N-(1-(7-(1-acetyl-2,5- dihydro-1H-pyrrol-3-yl)- 4-aminopyrrolo[2,1- f][1,2,4 ]triazin-5- yl)piperidin-3-yl)-4- methoxy-1-methyl-5-oxo- 2,5-dihydro-1H-pyrrole-3- carboxamide | 495 | B: 1.05 | (500 MHz, DMSO-d₆) δ 7.87 (d, J = 2.2 Hz, 1H), 6.77 (br d, J = 14.0 Hz, 1H), 6.72 (d, J = 10.2 Hz, 1H), 4.69 (br s, 1H), 4.49 (br s, 1H), 4.47 (br s, 1H), 4.27 (br s, 1H), 4.13 (s, 4H), 3.97 (s, 2H), 3.35 (br d, J = 9.6 Hz, 1H), 3.07 (br d, J = 8.3 Hz, 1H), 2.93 (s, 3H), 2.90-2.70 (m, 2H), 2.09- 1.96 (m, 3H), 1.88-1.70 (m, 3H), 1.64-1.43 (m, 1H) |
| 755 | 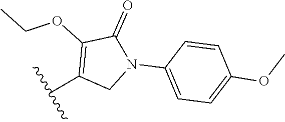 [SMILES: *C1=C(OCC)C(=O)N(c2ccc(OC)cc2)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- ethoxy-1-(4- methoxyphenyl)-5-oxo- 2,5-dihydro-1H-pyrrole-3- carboxamide | 601 | B: 1.44 | (500 MHz, DMSO-d₆) δ 7.87 (d, J = 2.1 Hz, 1H), 7.69 (br d, J = 9.2 Hz, 2H), 6.99 (br d, J = 8.9 Hz, 2H), 6.81-6.72 (m, 2H), 4.70 (br s, 1H), 4.67-4.58 (m, 2H), 4.51 (br s, 1H), 4.47 (s, 3H), 4.27 (br s, 1H), 4.19 (br dd, J = 5.8, 4.3 Hz, 1H), 3.77 (s, 3H), 3.11-3.01 (m, 1H), 2.98-2.79 (m, 3H), 2.09-1.99 (m, 3H), 1.83 (br s, 3H), 1.69-1.55 (m, 1H), 1.30 (t, J = 7.0 Hz, 3H) |
| 756 | 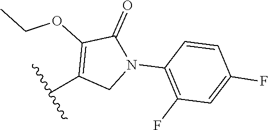 [SMILES: *C1=C(OCC)C(=O)N(c2ccc(F)cc2F)C1] | (R)-N-(1-(7-(1-acetyl-2,5- dihydro-1H-pyrrol-3-yl)- 4-aminopyrrolo[2,1- f\|[1,2,4]triazin-5- yl)piperidin-3-yl)-1-(2,4- difluorophenyl)-4-ethoxy- 5-oxo-2,5-dihydro-1H- pyrrole-3-carboxamide | 607 | B: 1.44 | (500 MHz, DMSO-d₆) δ 7.87 (d, J = 1.8 Hz, 1H), 7.67-7.60 (m, 1H), 7.47-7.40 (m, 1H), 7.20 (br t, J = 8.2 Hz, 1H), 6,78 (br d, J = 13.7 Hz, 1H), 6.73 (br d, J = 11.6 Hz, 1H), 4.70 (br s, 1H), 4.63- 4.55 (m, 2H), 4.50 (br s, 1H), 4.48-4.38 (m, 3H), 4.27 (br s, 1H), 4.17 (br d, J = 4.9 Hz, 1H), |
| | | | | | 3.36-3.29 (m, 1H), 3.12-3.02 |
| | | | | | (m, 1H), 2.97- 2.74 (m, 2H), 2.06 & |
| | | | | | 2.02 (s, 3H), 1.90-1.73 (m, |
| | | | | | 3H), 1.65-1.50 (m, 1H), 1.33-1.24 |
| | | | | | (m, 3H) |
| 757 | 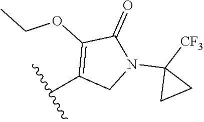 [SMILES: *C1=C(OCC)C(=O)N(C2(C(F)(F)F)CC2)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- ethoxy-5-oxo-1-(1- (trifluoromethyl)cyclopro- pyl)-2,5-dihydro-1H- pyrrole-3-carboxamide | 603 | B: 1.39 | (500 MHz, DMSO-d₆) δ 7.76 (br s, 1H), 6.78 (br d, J = 13.4 Hz, 1H), 6.73 (br d, J = 14.0 Hz, 1H), 4.69 (br s, 1H), 4.57-4.45 (m, 4H), 4.27 (br s, 1H), 4.17-4.07 (m, 3H), 3.28 (br d, J = 7.3 Hz, 1H), 3.11-3.01 (m, 1H), 2.96- 2.78 (m, 2H), 2.06 & 2.02 (s, 3H), 1.80 (br s, 3H), 1.63-1.48 (m, 1H), 1.36 (br s, 4H), 1.24 (br t, J = 6.4 Hz, 3H) |
| 758 | 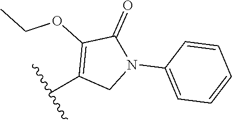 [SMILES: *C1=C(OCC)C(=O)N(c2ccccc2)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- ethoxy-5-oxo-1-phenyl- 2,5-dihydro-1H-pyrrole-3- carboxamide, TFA salt | 571 | B: 1.50 | (500 MHz, DMSO-d₆) δ 7.93 (s, 1H), 7.79 (br d, J = 8.2 Hz, 3H), 7.42 (t, J = 7.9 Hz, 2H), 7.19 (t, J = 7.5 Hz, 1H), 6.81 (s, 1H), 6.79- 6.76 (m, 1H), 4.70 (br s, 1H), 4.61 (br d, J = 6.7 Hz, 2H), 4.54- 4.46 (m, 4H), 4.28 (br s, 1H), 4.20 (br dd, J = 4.6, 2.4 Hz, 1H), |
| | | | | | 3.13-3.06 (m, 1H), 2.96 (br dd, |
| | | | | | J = 8.2, 3.4 Hz, 1H), 2.91-2.79 |
| | | | | | (m, 2H), 2.06 & 2.01 (s, 3H), 1.89- |
| | | | | | 1.78 (m, 3H), 1.59 (br dd, |
| | | | | | J = 8.1, 2.3 Hz, 1H), 1.30 |
| | | | | | (t, J = 7.0 Hz, 3H) |
| 759 | 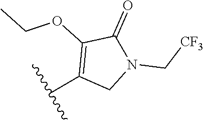 [SMILES: *C1=C(OCC)C(=O)N(CC(F)(F)F)C1] | (R)-N-(1-(7-(1-acetyl-2,5- dihydro-1H-pyrrol-3-yl)- 4-aminopyrrolo[2,1- f][1,2,4]triazin-5- yl)piperidin-3-yl)-4- ethoxy-5-oxo-1-(2,2,2- trifluoroethyl)-2,5- dihydro-1H-pyrrole-3- carboxamide | 577 | B: 1.32 | (500 MHz, DMSO-d₆) δ 7.86 (d, J = 1.8 Hz, 1H), 6.77 (br d, J = 13.7 Hz, 1H), 6.72 (d, J = 8.5 Hz, 1H), 4.69 (br s, 1H), 4.56 (q, J = 6.3 Hz, 2H), 4.49 (br s, 1H), 4.47 (br s, 1H), 4.30-4.20 (m, 3H), 4.18-4.13 (m, 1H), 4.12 (s, 2H), 3.11-3.00 (m, 1H), 2.97- 2.72 (m, 3H), 2.06 & 2.01 (m, 3H), 1.80 (br s, 3H), 1.64-1.47 (m, 1H), 1.25 (t, J = 7.0 Hz, 3H) |

### Example 760

### N—((R)-1-(7-(1-acetyl-2,5-dihydro-1H-pyrrol-3-yl)-4-aminopyrrolo[2,1-f][1,2,4]triazin-5-yl)piperidin-3-yl)-6-(((R)-1-methylpyrrolidin-3-yl)amino)picolinamide

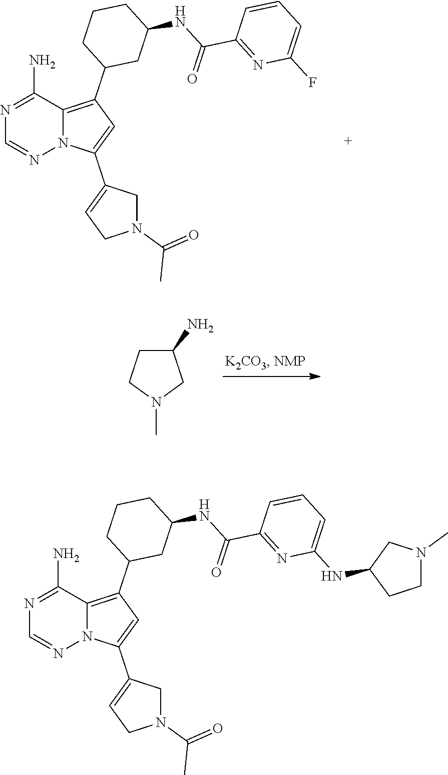

To a solution of Example 726 (30 mg, 0.065 mmol) in NMP (1 mL) was added (R)-1-methylpyrrolidin-3-amine (26 mg, 0.26 mmol) followed by addition of K₂CO₃ (27 mg, 0.19 mmol) at rt. The reaction was stirred under N₂ at 80° C. for 16 h. Reverse phase purification afforded Example 760 (10.1 mg, 28%). LCMS ESI m/z 545 (M+H)⁺, RT=0.98 min (Method B); ¹H NMR (500 MHz, DMSO-d₆) δ 7.84 (d, J=2.1 Hz, 1H), 7.51 (t, J=7.8 Hz, 1H), 7.15 (d, J=7.2 Hz, 1H), 6.90 (br d, J=3.0 Hz, 1H), 6.75 (br d, J=14.4 Hz, 1H), 6.70 (t, J=4.5 Hz, 1H), 6.65 (d, J=8.4 Hz, 1H), 4.67 (br s, 1H), 4.47 (br s, 1H), 4.45 (br s, 1H), 4.33 (br d, J=5.2 Hz, 1H), 4.25 (br d, J=1.7 Hz, 1H), 4.14 (br s, 1H), 3.18-3.11 (m, 1H), 2.98-2.73 (m, 3H), 2.56 (br s, 1H), 2.55 (s, 3H), 2.44-2.33 (m, 2H), 2.22-2.07 (m, 2H), 2.06-1.98 (m, 3H), 1.92-1.75 (m, 4H), 1.67-1.55 (m, 2H).

The following examples in Table 55 were prepared using a similar procedure as described in Example 760 by using the appropriate amines, prepared as intermediates described above, or obtained from commercial sources. Bases other than K₂CO₃, or solvents other than NMP may alternatively be used.

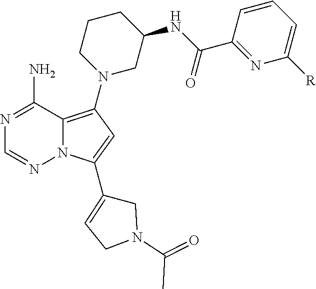

TABLE 55

| | | | | HPLC | |
|---|---|---|---|---|---|
| | | | | Meth- | |
| Ex- | | | LCMS | od | |
| am- | | | (M + | RT | |
| ple | R | Name | H)⁺ | (min.) | ¹H NMR |
| 761 | 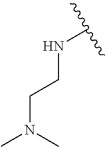 [SMILES: *NCCN(C)C] | (R)-N-(1-(7-(1- acetyl-2,5-dihydro- 1H-pyrrol-3- yl)-4-aminopyrrolo [2,1-f][1,2,4] triazin-5-yl)piperidin- 3-yl)-6-((2-(dimethyl- amino)ethyl)amino) picolinamide | 533 | B: 0.97 | (500 MHz, DMSO-d₆) δ 8.22 (br d, J = 6.1 Hz, 1H), 7.94 (s, 1H), 7.60 (t, J = 7.8 Hz, 1H), 7.25 (d, J = 7.2 Hz, 1H), 7.12 (br s, 1H), 6.80 (br, d, J = 7.2 Hz, 1H), 6.75 (br d, J = 8.9 Hz, 1H), 6.72 (br s, 1H), 4.69 (br d, J = 1.7 Hz, 1H), 4.49 (br d, J = 6.8 Hz, 2H), 4.28 (br s, 1H), 4.23-4.13 (m, 1H), 3.69 (br d, J = 4.0 Hz, 2H), 3.33-3.28 (m, 2H), 3.23-3.16 (m, 1H), 3.14-2.96 (m, 4H), 2.74 (br s, 3H), 2.03 (br d, J = 18.4 Hz, 3H), 1.96-1.82 (m, 7H), 1.65- 1.51 (m, 1H) |
| 763 | 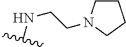 [SMILES: *NCCN1CCCC1] | (R)-N-(1-(7-(1-acetyl- 2,5-dihydro-1H- pyrrol-3-yl)-4-amino- pyrrolo[2,1-f][1,2,4] | 559 | B: 0.95 | (500 MHz, DMSO-d₆) δ 8.22 (br d, J = 6.1 Hz, 1H), 7.94 (s, 1H), 7.60 (t, J = 7.8 Hz, 1H), 7.25 (d, J = 7.2 Hz, 1H), 7.12 (br s, 1H), 6.80 (br d, J = 7.2 Hz, 1H), 6.75 (br d, J = 8.9 Hz, 1H), 6.72 (br s, 1H), 4.69 (br d, J = 1.7 Hz, 1H), 4.49 (br d, |
| | | triazin-5-yl)piperidin- | | | J = 6.8 Hz, 2H), 4.28 (br s, 1H), 4.23-4.13 (m, 1H), 3.69 (br |
| | | 3-yl)-6-((2- | | | d, J = 4.0 Hz, 2H), 3.33-3.28 (m, 2H), 3.23-3.16 |
| | | (pyrrolidin-1-yl)ethyl) | | | (m, 1H), 3.14-2.96 (m, 4H), 2.74 (br s, 3H), 2.03 (br d, |
| | | amino)picolinamide | | | 18.4 Hz, 3H), 1.96-1.82 (m, 7H), 1.65-1.51 (m, 1H) |
| 764 | 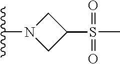 [SMILES: *N1CC(S(C)(=O)=O)C1] | (R)-N-(1-(7-(1-acetyl- 2,5-dihydro-1H- pyrrol-3-yl)-4- aminopyrrolo[2,1-f] [1,2,4]triazin-5-yl) | 580 | B: 1.36 | 500 MHz, DMSO-d₆) δ 8.28 (br s, 1H), 7.86 (s, 1H), 7.72 (br t, J = 7.8 Hz, 1H), 7.35 (br d, J = 7.1 Hz, 1H), 6.76 (br d, J = 12.6 Hz, 1H), 6.71 (br d, J = 8.9 Hz, 1H), 6.68 (br d, J = 8.1 Hz, 1H), 4.68 (br s, 1H), 4.48 (br s, 1H), 4.46 (br s, 1H), 4.42 (br d, J = 4.6 Hz, 1H), 4.32 (br t, J = 8.5 Hz, |
| | | piperidin-3-yl)-6-(3- | | | 2H), 4.28-4.20 (m, 3H), 4.16 (br d, J = 4.3 Hz, 1H), 3.21- |
| | | (methylsulfonyl) | | | 3.11 (m, 1H), 3.05 (s, 3H), 2.98 (br dd, J = 7.8, 3.0 Hz, 1H), |
| | | azetidin-1-yl) | | | 2.90-2.60 (m, 2H), 2.08-1.98 (m, 3H), 1.95- |
| | | picolinamide | | | 1.76 (m, 3H), 1.71-1.53 (m, 1H) |
| 765 | 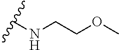 [SMILES: *NCCOC] | (R)-N-(1-(7-(1-acetyl- 2,5-dihydro-1H- pyrrol-3-yl)-4-amino- pyrrolo[2,1-f][1,2,4] | 520 | B: 1.45 | (500 MHz, DMSO-d₆) δ 8.24 (br s, 1H), 7.88 (d, J = 2.1 Hz, 1H), 7.51 (br t, J = 7.8 Hz, 1H), 7.15 (d, J = 6.7 Hz, 1H), 6.87-6.68 (m, 4H), 4.69 (br d, J = 1.2 Hz, 1H), 4.50 (br s, 1H), 4.47 (br d, J = 1.2 Hz, 1H), 4.27 (br d, J = 1.2 Hz, 1H), |
| | | triazin-5-yl)piperidin- | | | 4.22-4.14 (m, 1H), 3.91 (s, 3H), 3.85-3.71 (m, 2H), 3.50 (br |
| | | 3-yl)-6-((2-methoxy- | | | s, 3H), 3.23-3.09 (m, 1H), 3.05-2.93 (m, 1H), 2.88-2.64 |
| | | ethyl)amino) | | | (m, 2H), 2.05, 2.01 (s, 3H), 1.95-1.77 (m, 3H), 1.63-1.51 |
| | | picolinamide | | | (m, 1H) |
| 766 | 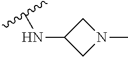 [SMILES: *NC1CN(C)C1] | (R)-N-(1-(7-(1- acetyl-2,5-dihydro- 1H-pyrrol-3-yl)-4- aminopyrrolo[2,1- | 531 | B: 0.91 | |
| | | f][1,2,4]triazin-5-yl) | | | |
| | | piperidin-3-yl)-6- | | | |
| | | ((1-methylazetidin-3- | | | |
| | | yl)amino)picolinamide | | | |
| 767 | 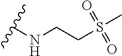 [SMILES: *NCCS(C)(=O)=O] | (R)-N-(1-(7-(1-acetyl- 2,5-dihydro-1H- pyrrol-3-yl)-4- aminopyrrolo[2,1-f] [1,2,4]triazin-5-yl) | 568 | B: 1.08 | (500 MHz, DMSO-d₆) δ 8.27 (br d, J = 7.4 Hz, 1H), 7.96 (s, 1H), 7.56 (t, J = 7.8 Hz, 1H), 7.22 (br d, J = 7.0 Hz, 1H), 6.81 (br s, 1H), 6.78 (br d, J = 8.0 Hz, 1H), 6.71 (d, J = 8.4 Hz, 1H), 4.69 (br s, 1H), 4.50 (br s, 1H), 4.47 (br s, 1H), 4.27 (br s, 1H), 4.23-4.11 (m, 1H), 3.78 (br |
| | | piperidin-3-yl)-6-((2- | | | s, 1H), 3.62-3.56 (m, 3H), 3.41-3.31 (m, 1H), 3.26- |
| | | (methylsulfonyl)ethyl) | | | 3.16 (m, 1H), 3.09-2.97 (m, 4H), 2.84-2.63 (m, |
| | | amino)picolinamide | | | 2H), 2.04 & 2.00 (s, 3H), 1.98-1.75 (m, 3H), |
| | | | | | 1.63-1.49 (m, 1H) |
| 768 | 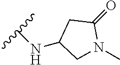 [SMILES: *NC1CC(=O)N(C)C1] | N-((R)-1-(7-(1-acetyl- 2,5-dihydro-1H- pyrrol-3-yl)-4-amino- pyrrolo[2,1-f][1,2,4] triazin-5-yl) | 559 | B: 1.19 | (500 MHz, DMSO-d₆) δ 8.30-8.13 (m, 1H), 7.95 (br d, J = 6.1 Hz, 1H), 7.56 (br t, J = 7.8 Hz, 1H), 7.22 (br d, J = 7.3 Hz, 1H), 6.84-6.75 (m, 2H), 6.68 (br d, J = 8.5 Hz, 1H), 4.70 (br s, 1H), 4.57 (br s, 1H), 4.51 (br s, 1H), 4.48 (br s, 1H), 4.28 (br s, 1H), 4.18 (br d, J = 2.4 Hz, |
| | | piperidin-3-yl)-6-((1- | | | 1H), 3.90 (s, 2H), 3.22 (br dd, J = 11.6, 8.5 Hz, 1H), |
| | | methyl-5-oxo- | | | 3.06-2.94 (m, 1H), 2.91-2.76 (m, 2H), 2.74 (s, |
| | | pyrrolidin-3-yl)amino) | | | 2H), 2.55 (s, 3H), 2.29-2.16 (m, 1H), 2.05 & 2.01 |
| | | picolinamide | | | (s,, 3H), 1.95-1.77 (m, 3H), 1.70-1.56 (m, 1H) |
| 769 | 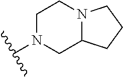 [SMILES: *N1CCN2CCCC2C1] | N-((R)-1-(7-(1-acetyl- 2,5-dihydro-1H- pyrrol-3-yl)-4-amino- pyrrolo[2,1-f][1,2,4] triazin-5-yl) piperidin-3-yl)-6- | 571 | B: 1.16 | (500 MHz, DMSO-d₆) δ 8.31-8.18 (m, 1H), 7.98 (s, 1H), 7.85-7.71 (m, 1H), 7.39 (br d, J = 5.2 Hz, 1H), 7.21-7.03 (m, 1H), 6.85-6.73 (m, 2H), 4.91-4.74 (m, 1H), 4.70 (br s, 1H), 4.51 (br s, 1H), 4.48 (br s, 1H), 4.28 (br s, 1H), 4.26-4.17 (m, 1H), 4.09-3.94 (m, 1H), 3.82-3.63 (m, 1H), 3.22-3.15 (m, 1H), 3.05 |
| | | (hexahydropyrrolo | | | (br d, J = 10.4 Hz, 1H), 2.89-2.71 (m, 2H), 2.55 (s, 4H), |
| | | [1,2-a]pyrazin-2(1H)- | | | 2.25-2.07 (m, 2H), 2.05 & 2.02 (s, 3H), |
| | | yl)picolinamide | | | 1.99-1.76 (m, 5H), 1.73-1.58 (m, 2H) |
| 770 | 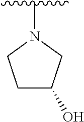 [SMILES: *N1CC[C@@H](O)C1] | N-((R)-1-(7-(1-acetyl- 2,5-dihydro-1H- pyrrol-3-yl)-4-amino- pyrrolo[2,1-f][1,2,4] triazin-5-yl) piperidin-3-yl)-6-((R)- 3-hydroxypyrrolidin- 1-yl)picolinamide | 532 | B: 1.20 | (500 MHz, DMSO-d₆) δ 7.88 (d, J = 2.4 Hz, 1H), 7.63 (t, J = 7.8 Hz, 1H), 7.20 (d, J = 7.0 Hz, 1H), 6.82-6.71 (m, 2H), 6.64 (br, d, J = 8.5 Hz, 1H), 4.69 (br s, 1H), 4.50 (br s, 1H), 4.47 (br s, 1H), 4.42 (br s, 1H), 4.27 (br s, 1H), 4.22-4.13 (m, 1H), 3.62--3.43 (m, 4H), 3.23- 3.12 (m, 1H), 3.06-2.93 (m, 1H), 2.89-2.63 (m, 2H), 2.16-1.98 (m, 4H), 1.91 (br d, J = 4.0 Hz, 2H), 1.84 (br d, J = 2.7 Hz, 2H), 1.68-1.53 (m, 1H) |
| 771 | 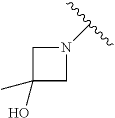 [SMILES: *N1CC(C)(O)C1] | (R)-N-(1-(7-(1-acetyl- 2,5-dihydro-1H- pyrrol-3-yl)-4-amino- pyrrolo[2,1-f][1,2,4] triazin-5-yl)piperidin- 3-yl)-6-(3-hydroxy- 3-methylazetidin-1- yl)picolinamide | 532 | B: 1.14 | (500 MHz, DMSO-d₆) δ 8.23 (br s, 2H), 7.87 (d, J = 2.1 Hz, 1H), 7.66 (t, J = 7.9 Hz, 1H), 7.28 (d, J = 7.3 Hz, 1H), 6.77 (br d, J = 13.4 Hz, 1H), 6.73 (br d, J = 13.4 Hz, 1H), 6.59 (br d, J = 8.2 Hz, 1H), 5.61 (s, 1H), 4.69 (br s, 1H), 4.50 (br s, 1H), 4.47 (br s, 1H), 4.27 (br s, 1H), 4.17 (dt, J = 5.4, 2.9 Hz, 1H), 3.95-3.87 (m, 2H), 3.83 (br dd, J = 7.9, 2.4 Hz, 2H), 3.21-3.10 (m, 1H), 3.04-2.93 (m, 1H), 2.88-2.63 (m, 2H), 2.05 & 2.01 (m, 3H), 1.91-1.75 |
| | | | | | (m, 3H), 1.68-1.53 (m, 1H), 1.46 (s, 3H) |
| 772 | 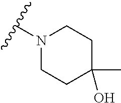 [SMILES: *N1CCC(C)(O)CC1] | (R)-N-(1-(7-(1-acetyl- 2,5-dihydro-1H- pyrrol-3-yl)-4-amino- pyrrolo[2,1-f][1,2,4] triazin-5-yl)piperidin- 3-yl)-6-(4-hydroxy-4- methylpiperidin-1- | 560 | B: 1.20 | (500 MHz, DMSO-d₆) δ 8.20 (br s, 1H), 7.87 (d, J = 2.1 Hz, 1H), 7.64 (t, J = 7.8 Hz, 1H), 7.23 (d, J = 7.0 Hz, 1H), 7.02 (br d, J = 8.5 Hz, 1H), 6.77 (br d, J = 12.8 Hz, 1H), 6.72 (d, J = 11.9 Hz, 1H), 4.69 (br s, 1H), 4.49 (br, s, 1H), 4.47 (br s, 1H), 4.26 (br s, 1H), 4.21-4.10 (m, 1H), 3.85 (br dd, J = 12.2, 4.3 Hz, 2H), 3.49-3.29 (m, 2H), 3.20-3.10 (m, 1H), 3.05-2.94 (m, 1H), 2.92-2.60 (m, 2H), 2.05 & 2.01 (s, 3H), |
| | | yl)picolinamide | | | 1.93-1.77 (m, 3H), 1.69-1.59 (m, 1H), 1.58-1.43 (m, 4H), |
| | | | | | 1.16 (s, 3H) |
| 773 | 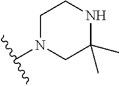 [SMILES: *N1CCNC(C)(C)C1] | (R)-N-(1-(7-(1-acetyl- 2,5-dihydro-1H- pyrrol-3-yl)-4- aminopyrrolo[2,1-f] [1,2,4]triazin-5-yl) piperidin-3-yl)-6-(3,3- | 559 | B: 1.16 | (500 MHz, DMSO-d₆) δ 9.08 (br s, 2H), 8.21 (br d, J = 7.6 Hz, 1H), 7.96 (s, 1H), 7.74 (t, J = 7.9 Hz, 1H), 7.35 (d, J = 7.3 Hz, 1H), 7.15 (br d, J = 8.5 Hz, 1H), 6.86-6.73 (m, 2H), 4.70 (br s, 1H), 4.51 (br s, 1H), 4.48 (br s, 1H), 4.28 (br s, 1H), 4.25-4.16 (m, 1H), 3.98-3.77 (m, 2H), 3.26 (br s, 2H), 3.21-3.14 (m, 1H), 3.04 (br d, J = 8.5 Hz, 1H), 2.91-2.71 (m, |
| | | dimethylpiperazin-1- | | | 2H), 2.05 & 2.01 (s, 3H), 1.94-1.80 (m, 3H), 1.66 (br |
| | | yl)picolinamide | | | s, 1H), 1.34 (br d, J = 4.0 Hz, 6H) |
| 774 | 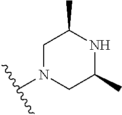 [SMILES: *N1C[C@@H](C)N[C@@H](C)C1] | N-((R)-1-(7-(1- acetyl-2,5-dihydro- 1H-pyrrol-3-yl)-4- aminopyrrolo[2,1-f] [1,2,4]triazin-5-yl) piperidin-3-yl)-6- ((3S,5R)-3,5- dimethylpiperazin-1- | 559 | B: 1.10 | (500 MHz, DMSO-d₆) δ 8.23 (br s, 1H), 7.87 (d, J = 2.1 Hz, 1H), 7.66 (t, J = 7.8 Hz, 1H), 7.25 (d, J = 7.3 Hz, 1H), 7.02 (br d, J = 8.2 Hz, 1H), 6.77 (br d, J = 12.8 Hz, 1H), 6.72 (d, J = 13.4 Hz, 1H), 4.69 (br s, 1H), 4.49 (br s, 1H), 4.47 (br s, 1H), 4.27 (br s, 1H), 4.18 (br d, J = 11.9 Hz, 3H), 3.05-2.92 (m, 1H), 2.83-2.80 (m, 1H), 2.91-2.80 (m, 1H), 2.79-2.69 (m, 2H), 2.30 (br t, J = 11.4 Hz, 2H), 2.05 & 2.01 (s, 3H), 1.90 (s, 2H), 1.87-1.75 (m, 3H), 1.74-1.60 (m, 1H), |
| | | yl)picolinamide | | | 1.10-0.89 (m, 6H) |
| 775 | 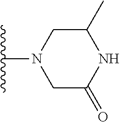 [SMILES: *N1CC(=O)NC(C)C1] | Desired product, N- ((R)-1-(7-(1-acetyl- 2,5-dihydro-1H- pyrrol-3-yl)-4-amino- pyrrolo[2,1-f][1,2,4] triazin-5-yl)piperidin- 3-yl)-6-(3-methyl-5- oxopiperazin-1-yl) | 559 | B: 1.12 | (500 MHz, DMSO-d₆) δ 8.29 (br d, J = 6.4 Hz, 1H), 8.19 (br s, 1H), 7.95 (s, 1H), 7.72 (br t, J = 7.9 Hz, 1H), 7.33 (d, J = 7.3 Hz, 1H), 7.07 (br d, J = 8.5 Hz, 1H), 6.86-6.76 (m, 2H), 4.70 (br s, 1H), 4.51 (br s, 1H), 4.48 (br d, J = 1.8 Hz, 1H), 4.36-4.19 (m, 3H), 4.11-3.88 (m, 2H), 3.60 (br dd, J = 7.3, 3.1 Hz, 1H), 3.28-3.14 (m, 2H), 3.03-2.95 (m, 1H), 2.93- 2.74 (m, 2H), 2.05 & 2.01 (s, 3H), 1.87 (br s, 3H), 1.74-1.62 (m, 1H), 1.15 (br d, J = 5.2 Hz, 3H) |
| | | picolinamide, 2 TFA | | | |
| 776 | 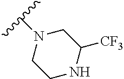 [SMILES: *N1CCNC(C(F)(F)F)C1] | N-((R)-1-(7-(1- acetyl-2,5-dihydro- 1H-pyrrol-3-yl)-4- aminopyrrolo[2,1-f] [1,2,4]triazin-5-yl) piperidin-3-yl)-6-(3- | 599 | B: 1.19 | (500 MHz, DMSO-d₆) δ 8.23 (br d, J = 8.5 Hz, 1H), 7.97 (s, 1H), 7.79 (t, J = 7.8 Hz, 1H), 7.40 (d, J = 7.3 Hz, 1H), 7.21 (br d, J = 8.2 Hz, 1H), 6.85-6.73 (m, 2H), 4.74-4.59 (m, 2H), 4.51 (br s, 1H), 4.48 (br s, 1H), 4.36 (br d, J = 11.0 Hz, 1H), 4.31-4.14 (m, 3H), 3.36-3.15 (m, 3H), 3.06 (br d, J = 1.8 Hz, 2H), 2.90-2.71 (m, 2H), 2.05 & 2.01 (s, 3H), 1.96-1.78 (m, 3H), 1.69-1.54 (m, |
| | | (trifluoromethyl) | | | 1H) |
| | | piperazin-1-yl) | | | |
| | | picolinamide | | | |
| 777 | 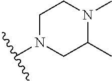 [SMILES: *N1CCN(C)C(C)C1] | N-((R)-1-(7-(1-acetyl- 2,5-dihydro-1H- pyrrol-3-yl)-4- aminopyrrolo[2,1-f] [1,2,4]triazin-5-yl) piperidin-3-yl)-6-(3,4- | 559 | B: 0.98 | (500 MHz, DMSO-d₆) δ 8.27 (br s, 1H), 7.85 (s, 1H), 7.68 (br t, J = 7.9 Hz, 1H), 7.28 (br d, J = 7.0 Hz, 1H), 7.03 (br d, J = 8.5 Hz, 1H), 6.77 (br d, J = 12.2 Hz, 1H), 6.71 (br d, J = 5.8 Hz, 1H), 4.68 (br s, 1H), 4.49 (br s, 1H), 4.47 (br s, 1H), 4.27 (br d, J = 1.2 Hz, 1H), 4.22-4.09 (m, 2H), 4.09-4.00 (m, 1H), 3.74-3.68 (m, 2H), 3.18-3.08 (m, 1H), 3.01-2.90 (m, 2H), 2.85- |
| | | dimethylpiperazin-1- | | | 2.73 (m, 2H), 2.59 (br s, 2H), 2.19 (s, 3H), 2.05 & 2.01 |
| | | yl)picolinamide | | | (s, 3H), 1.83-1.76 (m, 3H), 1.71-1.60 (m, 1H), |
| | | | | | 1.02 (br d, J = 5.5 Hz, 3H) |
| 778 | 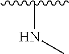 [SMILES: *NC] | (R)-N-(1-(7-(1-acetyl- 2,5-dihydro-1H- pyrrol-3-yl)-4-amino- pyrrolo[2,1-f][1,2,4] | 476 | B: 1.48 | (500 MHz, DMSO-d₆) δ 8.30 (br s, 1H), 7.86 (d, J = 2.1 Hz, 1H), 7.52 (br t, J = 7.6 Hz, 1H), 7.15 (d, J = 7.0 Hz, 1H), 6.77 (br d, J = 13.7 Hz, 1H), 6.73 (br d, J = 10.7 Hz, 1H), 6.64 (br d, J = 8.2 Hz, 1H), 4.69 (br s, 1H), 4.49 (br s, 1H), 4.47 (br s, 1H), 4.27 (br s, |
| | | triazin-5-yl)piperidin- | | | 1H), 4.21-4.08 (m, 1H), 3.18 (br s, 1H), 3.04-2.91 (m, 1H), 2.83 |
| | | 3-yl)-6- | | | (br d, J = 4.3 Hz, 2H), 2.78 (m, 21H), 2.05 & 2.01 (s, 3H), |
| | | (methylamino) | | | 1.94-1.75 (m, 3H), 1.67-1.50 (m, 1H) |
| | | picolinamide | | | |
| 779 | 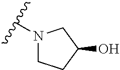 [SMILES: *N1CC[C@H](O)C1] | N-((R)-1-(7-(1-acetyl- 2,5-dihydro-1H- pyrrol-3-yl)-4- aminopyrrolo[2,1-f] [1,2,4]triazin-5-yl) | 532 | B: 1.06 | (500 MHz, DMSO-d₆) δ 7.86 (d, J = 2.1 Hz, 1H), 7.63 (t, J = 7.8 Hz, 1H), 7.20 (d, J = 7.0 Hz, 1H), 6.77 (br d, J = 13.7 Hz, 1H), 6.73 (d, J = 9.5 Hz, 1H), 6.63 (br d, J = 8.5 Hz, 1H), 4.68 (br s, 1H), 4.49 (br s, 1H, 4.46 (br s, 1H), 4.41 (br d, J = 1.2 Hz, 1H), 4.27 (br s, 1H), 4.21-4.11 (m, 1H), 3.49 (m, 4H), 3.17 (br dd, |
| | | piperidin-3-yl)-6-((S)- | | | J = 9.8, 5.8 Hz, 1H), 3.04-2.89 (m, 1H), 2.88-2.61 (m, 2H), |
| | | 3-hydroxypyrrolidin- | | | 2.10-1.97 (m, 4H), 1.97-1.87 (m, 2H), |
| | | 1-yl)picolinamide | | | 1.82 (br d, J = 3.4 Hz, 2H), 1.69-1.53 (m, 1H) |
| 780 | 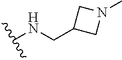 [SMILES: *NCC1CN(C)C1] | (R)-N-(1-(7-(1-acetyl- 2,5-dihydro-1H-pyrrol- 3-yl)-4-amino- pyrrolo[2,1-f][1,2,4] triazin-5-yl)piperidin- | 545 | B: 1.03 | (500 MHz, DMSO-d₆) δ 7.88 (s, 1H), 7.55 (br t, J = 8.1 Hz, 1H), 7.20 (br d, J = 6.7 Hz, 1H), 6.78 (br d, J = 8.9 Hz, 1H), 6.70 (br dd, J = 10.4, 7.9 Hz, 2H), 4.69 (br s, 1H), 4.49 (br s, 1H), 4.48 (br s, 1H), 4.27 (br s, 1H), 4.17 (br s, 2H), 4.06-3.98 (m, 1H), 3.97-3.88 (m, 1H), 3.81-3.72 (m, 1H), 3.45-3.36 (m, 1H), 3.22- |
| | | 3-yl)-6-(((1-methyl- | | | 3.14 (m, 1H), 3.06 (br s, 1H), 3.00 (s, 3H), 2.86-2.73 (m, 4H), |
| | | azetidin-3-yl)methyl) | | | 2.03 (br d, J = 19.8 Hz, 3H), 1.95-1.89 (m, 1H), |
| | | amino)picolinamide | | | 1.83 (br s, 2H), 1.66-1.59 (m, 1H) |
| 781 | 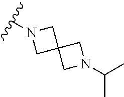 [SMILES: *N1CC2(C1)CN(C(C)C)C2] | (R)-N-(1-(7-(1-acetyl- 2,5-dihydro-1H- pyrrol-3-yl)-4-amino- pyrrolo[2,1-f][1,2,4] triazin-5-yl) piperidin-3-yl)-6-(6- isopropyl-2,6- | 585 | B: 1.06 | (500 MHz, DMSO-d₆) δ 8.24 (br dd, J = 6.1, 2.1 Hz, 1H), 7.98 (s, 1H), 7.70 (t, J = 7.9 Hz, 1H), 7.32 (d, J = 7.3 Hz, 1H), 6.82 (s, 1H), 6.80 (s, 1H), 6.64 (d, J = 7.9 Hz, 1H), 4.69 (br s, 1H), 4.50 (br s, 1H), 4.48 (br s, 1H), 4.35-4.16 (m, 8H), 4.16-4.07 (m, 2H), 3.45- 3.35 (m, 1H), 3.24-3.14 (m, 1H), 3.02 (dt, J = 5.9, 3.0 Hz, 1H), 2.88-2.71 (m, 2H), 2.03 (d, J = 19.5 Hz, 3H), 1.95-1.76 (m, 3H), 1.59 (br s, 1H), 1.13 (br d, J = 6.4 Hz, 6H) |
| | | diazaspiro[3.3]heptan- | | | |
| | | 2-yl)picolinamide | | | |
| 782 | 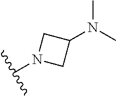 [SMILES: *N1CC(N(C)C)C1] | (R)-N-(1-(7-(1-acetyl- 2,5-dihydro-1H- pyrrol-3-yl)-4- aminopyrrolo[2,1-f] [1,2,4]triazin-5-yl) piperidin-3-yl)-6-(3- (dimethylamino) | 545 | B: 1.02 | (500 MHz, DMSO-d₆) δ 8.23 (br s, 1H), 7.87 (d, J = 2.1 Hz, 1H), 7.66 (t, J = 7.5 Hz, 1H), 7.27 (d, J = 7.3 Hz, 1H), 6.77 (br d, J = 13.1 Hz, 1H), 6.72 (d, J = 13.1 Hz, 1H), 6.58 (br d, J = 8.2 Hz, 1H), 4.69 (br s, 1H), 4.49 (br s, 1H), 4.47 (br s, 1H), 4.26 (br s, 1H), 4.21-4.11 (m, 1H), 4.09-4.00 (m, 2H), 3.84-3.73 (m, 2H), 3.25-3.12 (m, 2H), 3.05-2.91 (m, 1H), 2.88-2.65 (m, 2H), 2.13 (s, 6H), 2.07-1.98 (m, 3H), 1.90-1.76 (m, 3H), 1.68-1.53 (m, 1H) |
| | | azetidin-1-yl) | | | |
| | | picolinamide | | | |
| 783 | 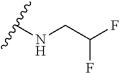 [SMILES: *NCC(F)F] | (R)-N-(1-(7-(1- acetyl-2,5-dihydro- 1H-pyrrol-3-yl)-4- aminopyrrolo[2,1-f] [1,2,4]triazin-5-yl) piperidin-3-yl)-6- | 526 | B: 1.24 | (500 MHz, DMSO-d₆) δ 8.23 (br s, 1H), 7.87 (d, J = 1.8 Hz, 1H), 7.58 (br t, J = 7.6 Hz, 1H), 7.23 (br d, J = 7.3 Hz, 1H), 7.17 (br s, 1H), 6.78 (br d, J = 8.9 Hz, 2H), 6.72 (d, J = 13.4 Hz, 1H), 6.29- 6.01 (m, 1H), 4.69 (br s, 1H), 4.49 (br s, 1H), 4.47 (br s, 1H), 4.27 (br d, J = 1.2 Hz, 1H), 4.16 (br d, J = 2.7 Hz, 1H), 3.93- 3.74 (m, 2H), 3.22-3.11 (m, 1H), 3.00 (s, 1H), 2.90-2.61 (m, |
| | | ((2,2- | | | 2H), 2.09-1.97 (m, 3H), 1.95-1.76 (m, 3H), 1.68-1.52 (m, 1H) |
| | | difluoroethyl) | | | |
| | | amino)picolinamide | | | |
| 784 | 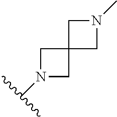 [SMILES: *N1CC2(C1)CN(C)C2] | (R)-N-(1-(7-(1-acetyl- 2,5-dihydro-1H- pyrrol-3-yl)-4- aminopyrrolo[2,1-f] [1,2,4]triazin-5-yl) piperidin-3-yl)-6- (6-methyl-2,6- diazaspiro[3.3]heptan- | 557 | B: 1.00 | (500 MHz, DMSO-d₆) δ 8.26 (br s, 1H), 7.86 (d, J = 1.5 Hz, 1H), 7.65 (br t, J = 7.6 Hz, 1H), 7.27 (d, J = 7.0 Hz, 1H), 6.77 (br d, J = 13.4 Hz, 1H), 6.72 (d, J = 7.9 Hz, 1H), 6.57 (br d, J = 8.2 Hz, 1H), 4.68 (br s, 1H), 4.49 (br s, 1H), 4.47 (br s, 1H), 4.27 (br s, 1H), 4.22-4.11 (m, 1H), 4.05 (s, 4H), 3.35 (br s, 4H), 3.17-3.12 (m, 1H), 3.03-2.91 (m, 1H), 2.90-2.65 (m, 2H), 2.23 (s, 3H), 2.09-1.99 (m, 3H), 1.82 (br s, 3H), 1.70-1.53 (m, 1H) |
| | | 2-yl)picolinamide | | | |

### Example 785

### (R)—N-(1-(4-amino-7-(6-cyanopyridin-3-yl)pyrrolo[2,1-f][1,2,4]triazin-5-yl)piperidin-3-yl)-6-(4-methylpiperazin-1-yl)picolinamide

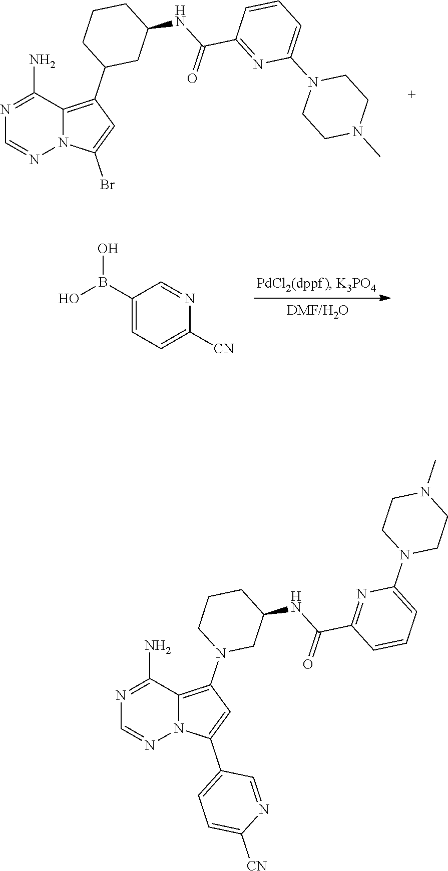 [SMILES: CN1CCN(c2cccc(C(=O)N[C@@H]3CCCC(c4cc(Br)n5ncnc(N)c45)C3)n2)CC1 ; N#Cc1ccc(B(O)O)cn1 ; CN1CCN(c2cccc(C(=O)N[C@@H]3CCCN(c4cc(-c5ccc(C#N)nc5)n5ncnc(N)c45)C3)n2)CC1]

A pressure vial charged with Intermediate 62 (30 mg, 0.058 mmol), (6-cyanopyridin-3-yl)boronic acid (17 mg, 0.117 mmol), K₃PO₄ (25 mg, 117 mmol) and PdCl₂(dppf) (9 mg, 0.012 mmol) was degassed and filled with N₂, and then DMF (1 mL) and H₂O (0.2 mL) were added. The reaction was heated at 70° C. for 1 h. Reverse phase HPLC purification gave Example 785 (17.3 mg, 55%). LCMS ESI m/z 538 (M+H)⁺. RT=1.10 min (Method B); ¹H NMR (500 MHz, DMSO-d₆) δ 9.41 (s, 1H), 8.83-8.73 (m, 1H), 8.32-8.20 (m, 1H), 8.06 (br d, J=8.2 Hz, 1H), 7.94 (s, 1H), 7.69 (t, J=7.9 Hz, 1H), 7.37 (s, 1H), 7.30 (d, J=7.0 Hz, 1H), 7.02 (br d, J=8.5 Hz, 1H), 4.19 (br d, J=4.0 Hz, 1H), 3.54 (br s, 4H), 3.24-3.15 (m, 1H), 3.09-2.98 (m, 1H), 2.95-2.72 (m, 2H), 2.44 (br s, 4H), 2.24 (s, 3H), 1.96-1.80 (m, 3H), 1.74-1.60 (m, 1H).

The following examples in Table 56 were prepared using a similar procedure as described in Example 785 by coupling Intermediate 62 with the appropriate boronic acids or boronates, prepared as intermediates described above, or obtained from commercial sources. Palladium catalysts other than PdCl₂(dppf) and solvents, such as dioxane/H₂O, THF/H₂O, can alternatively be used.

TABLE 56

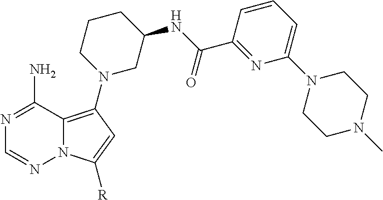

| | | | | HPLC | |
|---|---|---|---|---|---|
| | | | | Method, | |
| Ex- | | | LCMS | RT | |
| ample | R | Name | (M + H)⁺ | (min.) | ¹H NMR |
| 786 | 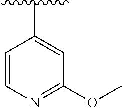 [SMILES: *c1ccnc(OC)c1] | (R)-N-(1-(4-amino-7- (2-methoxypyridin-4- yl)pyrrolo[2,1-f][1,2, 4]triazin-5-yl)piperidin- 3-yl)-6-(4- methylpiperazin-1-yl) picolinamide | 543 | B: 120 | (500 MHz, DMSO-d₆) δ 8.29 (br d, J = 8.2 Hz, 1H), 8.21 (d, J = 5.2 Hz, 1H), 8.02 (s, 1H), 7.78 (t, J = 7.9 Hz, 1H), 7.66-7.58 (m, 2H), 7.41 (d, J = 7.3 Hz, 1H), 7.32 (s, 1H), 7.14 (d, J = 8.9 Hz, 1H), 4.54 (br s, 1H), 4.32-4.16 (m, 1H), 3.89 (s, 3H), 3.63 (br dd, J = 3.7, 1.8 Hz, 1H), 3.22 (br d, J = 8.5 Hz, 1H), 3.13-3.04 (m, 1H), 2.93 (br s, 1H), 2.86 (s, 3H), 2.83-2.73 (m, 1H), 2.55 (s, 6H), 1.98-1.80 (m, 3H), 1.74-1.61 (m, 1H) |
| 787 | 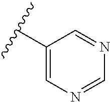 [SMILES: *c1cncnc1] | (R)-N-(1-(4-amino-7- (pyrimidin-5-yl) pyrrolo[2,1-f][1,2,4] triazin-5-yl)piperidin- 3-yl)-6-(4-methyl- piperazin-1-yl) picolinamide | 514 | B: 0.98 | (500 MHz, DMSO-d₆) δ 9.43 (s, 2H), 9.11 (s, 1H), 8.30 (br d, J = 8.5 Hz, 1H), 7.97 (s, 1H), 7.78 (t, J = 7.9 Hz, 1H), 7.41 (d, J = 7.3 Hz, 1H), 7.31 (s, 1H), 7.14 (d, J = 8.5 Hz, 1H), 4.31-4.15 (m, 1H), 3.69-3.47 (m, 2H), 3.22 (br d, J = 7.9 Hz, 1H), 3.13-3.05 (m, 1H), 2.95- 2.74 (m, 5H), 3.2-2.5 9m, 6H), 1.98-1.80 (m, 3H), 1.74-1.57 (m, 1H) |
| 788 | 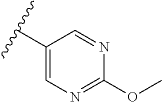 [SMILES: *c1cnc(OC)nc1] | (R)-N-(1-(4-amino-7- (2-methoxypyrimidin- 5-yl)pyrrolo[2,1-f] [1,2,4]triazin-5-yl) piperidin-3-yl)-6-(4- methylpiperazin-1- yl)picolinamide | 544 | B: 1.10 | (500 MHz, DMSO-d₆) δ 9.21 (s, 2H), 8.32-8.20 (m, 1H), 7.86 (s, 1H), 7.68 (t, J = 7.8 Hz, 1H), 7.30 (d, J = 7.3 Hz, 1H), 7.13 (s, 1H), 7.02 (d, J = 8.5 Hz, 1H), 4.28-4.12 (m, 1H), 3.97 (s, 3H), 3.53 (br s, 4H), 3.25-3.15 (m, 1H), 3.08-2.96 (m, 1H), 2.94-2.72 (m, 2H), 2.41 (br s, 4H), 2.22 (s, 3H), 1.92-1.82 (m, 3H), 1.74-1.60 (m, 1H) |
| 789 | 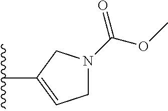 [SMILES: *C1=CCN(C(=O)OC)C1] | methyl (R)-3-(4-amino- 5-(3-(6-(4-methyl- piperazin-1-yl) picolinamido)piperidin- 1-yl)pyrrolo[2,1-f] [1,2,4]triazin-7-yl)-2,5- dihydro-1H-pyrrole-1- carboxylate | 560 | B: 1.10 | (500 MHz, DMSO-d₆) δ 8.23 (br s, 1H), 7.85 (s, 1H), 7.68 (t, J = 7.8 Hz, 1H), 7.29 (d, J = 7.0 Hz, 1H), 7.02 (br d, J = 8.5 Hz, 1H), 6.72 (br s, 2H), 4.54 (br s, 1H), 4.51 (br s, 1H), 4.34-4.23 (m, 2H), 4.15 (br dd, J = 7.3, 4.3 Hz, 1H), 3.65 (s, 3H), 3.55 (br s, 4H), 3.17-3.10 (m, 1H), 3.02-2.92 (m, 1H), 2.88-2.67 (m, 2H), 2.42 (br s, 4H), 2.23 (s, 3H), 1.90-1.75 (m, 3H), 1.71-1.56 (m, 1H) |
| 790 | 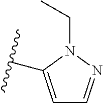 [SMILES: *c1ccnn1CC] | (R)-N-(1-(4-amino-7- (1-ethyl-1H-pyrazol- 5-yl)pyrrolo[2,1-f][1,2, 4]triazin-5-yl) piperidin-3-yl)-6-(4- methylpiperazin-1- yl)picolinamide | 530 | B: 1.12 | (500 MHz, DMSO-d₆) δ 8.29-8.17 (m, 1H), 7.77 (s, 1H), 7.68 (t, J = 8.0 Hz, 1H), 7.53 (d, J = 1.7 Hz, 1H), 7.29 (d, J = 7.1 Hz, 1H), 7.01 (d, J = 8.7 Hz, 1H), 6.79 (s, 1H), 6.53 (d, J = 1.7 Hz, 1H), 4.24-4.14 (m, 1H), 4.06 (q, J = 7.1 Hz, 2H), 3.75-3.65 (m, 4H), 3.18 (br d, J = 11.9 Hz, 1H), 3.06-2.96 (m, 1H), 2.94-2.72 (m, 2H), 2.41 (br t, J = 4.6 Hz, 4H), 2.21 (s, 3H), 1.90-1.75 (m, |
| | | | | | 3H), 1.71-1.58 (m, 1H), 1.25 (t, J = 7.2 Hz, 3H) |
| 791 | 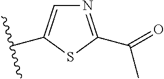 [SMILES: *c1cnc(C(C)=O)s1] | (R)-N-(1-(7-(2-acetyl- thiazol-5-yl)-4-amino- pyrrolo[2,1-f][1,2,4] triazin-5-yl)piperidin- 3-yl)-6-(4-methyl- piperazin-1-yl) picolinamide | 561 | B: 1.25 | (500 MHz, DMSO-d₆) δ 8.69-8.62 (m, 1H), 8.25 (br d, J = 7.5 Hz, 1H), 8.03 (s, 1H), 7.69 (t, J = 7.9 Hz, 1H), 7.38 (s, 1H), 7.31 (d, J = 7.2 Hz, 1H), 7.03 (br d, J = 8.7 Hz, 1H), 4.28-4.13 (m, 1H), 3.54 (br s, 4H), 3.26-3.17 (m, 1H), 3.10-3.01 (m, 1H), 2.92-2.84 (m, 1H), 2.83- 2.74 (m, 1H), 2.63 (s, 3H), 2.51 (br s, 4H), 2.27 (s, 3H), 1.97-1.79 (m, 3H), 1.75-1.60 (m, 1H) |
| 792 | 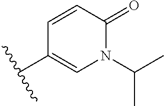 [SMILES: *c1ccc(=O)n(C(C)C)c1] | (R)-N-(1-(4-amino-7- (1-isopropyl-6-oxo-1,6- dihydropyridin-3-yl) pyrrolo[2,1-f][1,2,4] triazin-5-yl)piperidin- 3-yl)-6-(4-methyl- piperazin-1-yl) | 571 | B: 1.04 | (500 MHz, DMSO-d₆) δ 8.54 (d, J = 2.5 Hz, 1H), 8.31- 8.18 (m, 1H), 7.98 (dd, J = 9.3, 2.2 Hz, 1H), 7.83 (s, 1H), 7.68 (dd, J = 8.4, 7.4 Hz, 1H), 7.30 (d, J = 7.2 Hz, 1H), 7.02 (br d, J = 8.5 Hz, 1H), 6.96 (s, 1H), 6.49 (d, J = 9.5 Hz, 1H), 5.17-5.04 (m, 1H), 4.25-4.11 (m, 1H), 3.57-3.55 (m, 4H), 3.22-3.14 (m, 1H), 3.06-2.96 (m, 1H), 2.93-2.68 (m, 2H), 2.45 (br s, 4H), 2.24 (s, 3H), |
| | | picolinamide | | | 1.91-1.76 (m, 3H), 1.72-1.60 (m, 1H), 1.34 (br d, J = 6.7 |
| | | | | | Hz, 6H) |
| 793 | 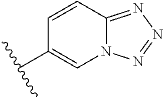 [SMILES: *c1ccc2nnnn2c1] | (R)-N-(1-(4-amino-7- (tetrazolo[1,5-a] pyridin-6-yl)pyrrolo [2,1-f][1,2,4]triazin-5- yl)piperidin-3-yl)-6-(4- methylpiperazin-1- yl)picolinamide | 554 | B: 1.02 | (500 MHz, DMSO-d₆) δ 10.10 (s, 1H), 8.49 (br d, J = 9.5 Hz, 1H), 8.35-8.23 (m, 2H), 8.05 (s, 1H), 7.78 (t, J = 7.9 Hz, 1H), 7.44 (s, 1H), 7.43-7.37 (m, 1H), 7.15 (d, J = 8.9 Hz, 1H), 4.32-4.17 (m, 1H), 3.47 (m, 4H, integration reduced by saturation), 3.28-3.21 (m, 1H), 3.15-3.04 (m, 1H), 2.87 (s, 3H), 2.97-2.73 (m, 2H), 2.55 (s, 4H), 1.99-1.83 (m, 3H), 1.77-1.64 (m, 1H) |
| 794 | 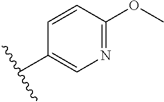 [SMILES: *c1ccc(OC)nc1] | (R)-N-(1-(4-amino-7- (6-methoxypyridin-3- yl)pyrrolo[2,1-f][1,2,4] triazin-5-yl)piperidin- 3-yl)-6-(4-methyl- piperazine-1-yl) picolinamide | 543 | B: 1.15 | (500 MHz, DMSO-d₆) δ 8.81 (d, J = 1.5 Hz, 1H), 8.35 (dd, J = 8.5, 2.4 Hz, 1H), 8.29-8.20 (m, 1H), 7.83 (s, 1H), 7.69 (t, J = 7.9 Hz, 1H), 7.31 (d, J = 7.3 Hz, 1H), 7.07-6.98 (m, 2H), 6.92 (d, J = 8.5 Hz, 1H), 4.25-4.13 (m, 1H), 3.90 (s, 3H), 3.56 (br s, 4H), 3.25-3.14 (m, 1H), 3.08-2.98 (m, 1H), 2.94-2.66 (m, 2H), 2.43 (br s, 4H), 2.24 (s, 3H), 1.93-1.78 (m, 3H), 1.74-1.59 (m, 1H) |
| 795 | 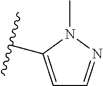 [SMILES: *c1ccnn1C] | (R)-N-(1-(4-amino-7- (1-methyl-1H-pyrazol- 5-yl)pyrrolo[2,1-f] [1,2,4]triazin-5-yl) piperidin-3-yl)-6-(4- methylpiperazin-1- yl)picolinamide | 516 | B: 1.00 | (500 MHz, DMSO-d₆) δ 8.29-8.13 (m, 1H), 7.81 (s, 1H), 7.70 (t, J = 7.9 Hz, 1H), 7.50 (d, J = 1.6 Hz, 1H), 7.31 (d, J = 7.1 Hz, 1H), 7.04 (d, J = 8.6 Hz, 1H), 6.87 (s, 1H), 6.63 (d, J = 1.6 Hz, 1H), 4.28-4.09 (m, 1H), 3.83 (s, 3H), 3.55-3.50 (m, 4H), 3.24-3.16 (m, 1H), 3.08-2.98 (m, 1H), 2.95-2.69 (m, 2H), 2.60-2.55 (m, 4H), 2.32 (br s, 3H), 1.91-1.78 (m, 3H), 1.73-1.59 (m, 1H) |
| 796 | 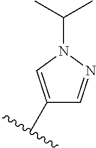 [SMILES: *c1cnn(C(C)C)c1] | (R)-N-(1-(4-amino-7- (1-isopropyl-1H- pyrazol-5-yl)pyrrolo [2,1-f][1,2,4]triazin-5- yl)piperidin-3-yl)-6-(4- methylpiperazin-1-yl) picolinamide | 544 | B: 1.51 | (500 MHz, DMSO-d₆) δ 8.35 (s, 1H), 8.25 (br d, J = 7.9 Hz, 1H), 8.04 (s, 1H), 7.82 (s, 1H), 7.69 (t, J = 7.9 Hz, 1H), 7.30 (d, J = 7.3 Hz, 1H), 7.03 (d, J = 8.5 Hz, 1H), 6.87 (s, 1H), 4.56 (dt, J = 13.3, 6.5 Hz, 1H), 4.25-4.09 (m, 1H), 3.64-3.35 (m, 4H), 3.23-3.11 (m, 1H), 3.07- 2.95 (m, 1H), 2.91-2.70 (m, 2H), 2.44 (br s, 4H), 2.24 (s, 3H), 1.90-1.77 (m, 3H), 1.73-1.60 (m, 1H), 1.44 (d, J = 6.7 Hz, 6H) |
| 797 | 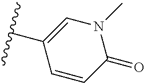 [SMILES: *c1ccc(=O)n(C)c1] | (R)-N-(1-(4-amino-7- (1-methyl-6-oxo-1,6- dihydropyridin-3-yl) pyrrolo[2,1-f][1,2,4] triazin-5-yl)piperidin- 3-yl)-6-(4-methyl- | 543 | B: 1.26 | (500 MHz, DMSO-d₆) δ 8.50 (d, J = 2.1 Hz, 1H), 8.28 (br d, J = 8.5 Hz, 1H), 8.04 (dd, J = 9.3, 2.6 Hz, 1H), 7.96 (s, 1H), 7.78 (t, J = 7.9 Hz, 1H), 7.41 (d, J = 7.3 Hz, 1H), 7.15 (d, J = 8.5 Hz, 1H), 6.98 (s, 1H), 6.52 (d, J = 9.5 Hz, 1H), 4.34-4.17 (m, 1H), 3.22 (br d, J = 7.6 Hz, 1H), 3.10 (br d, J = 10.7 Hz, 1H), 2.92-2.81 (m, 4H), |
| | | piperazin-1-yl) | | | 2.81-2.71 (m, 1H), 2.51 (br s, 4H), 1.98-1.80 (m, 3H), |
| | | picolinamide | | | 1.74-1.62 (m, 1H). signals of N—Me on pyridone and |
| | | | | | 4H on piperazine were invisible due to water |
| | | | | | suppression. |
| 798 | 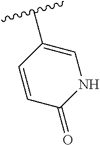 [SMILES: *c1ccc(=O)[nH]c1] | (R)-N-(1-(4-amino-7- (6-oxo-1,6-dihydro- pyridin-3-yl)pyrrolo [2,1-f][1,2,4]triazin-5- yl)piperidin-3-yl)-6-(4- methylpiperazin-1- yl)picolinamide | 529 | B: 0.98 | (500 MHz, DMSO-d₆) δ 8.35 (br s, 1H), 8.25 (br s, 1H), 8.01 (dd, J = 9.6, 2.4 Hz, 1H), 7.81 (br s, 1H), 7.68 (t, J = 7.8 Hz, 1H), 7.29 (d, J = 7.4 Hz, 1H), 7.01 (br d, J = 8.4 Hz, 1H), 6.93 (s, 1H), 6.46 (br d, J = 9.5 Hz, 1H), 4.24-4.07 (m, 1H), 3.62 (br s, 4H), 3.16 (br d, J = 7.5 Hz, 1H), 3.05-2.94 (m, 1H), 2.91-2.63 (m, 2H), 2.41 (br s, 4H), 2.21 (s, 3H), 1.95-1.75 (m, 3H), 1.72-1.59 (m, 1H) |
| 799 | 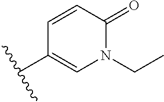 [SMILES: *c1ccc(=O)n(CC)c1] | (R)-N-(1-(4-amino-7- (1-ethyl-6-oxo-1,6- dihydropyridin-3-yl) pyrrolo[2,1-f][1,2,4] triazin-5-yl)piperidin- 3-yl)-6-(4-methyl- piperazine-1-yl) | 557 | B: 0.98 | (500 MHz, DMSO-d₆) δ 8.47 (d, J = 2.1 Hz, 1H), 8.25 (br d, J = 7.9 Hz, 1H), 8.03 (dd, J = 9.5, 2.4 Hz, 1H), 7.81 (s, 1H), 7.68 (dd, J = 8.5, 7.3 Hz, 1H), 7.30 (d, J = 7.0 Hz, 1H), 7.02 (d, J = 8.5 Hz, 1H), 6.91 (s, 1H), 6.50 (d, J = 9.5 Hz, 1H), 4.25-4.11 (m, 1H), 3.98 (q, J = 7.1 Hz, 2H), 3.64-3.48 (m, 4H), 3.18 (br d, J = 7.0 Hz, 1H), 3.06-2.96 (m, 1H), 2.91-2.68 (m, |
| | | picolinamide | | | 2H), 2.43 (br s, 4H), 2.23 (s, 3H), 1.91-1.76 (m, |
| | | | | | 3H), 1.72-1.59 (m, 1H), 1.26 (t, J = 7.0 Hz, 3H) |
| 800 | 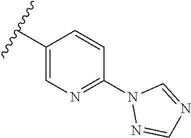 [SMILES: *c1ccc(-n2cncn2)nc1] | (R)-N-(1-(7-(6-(1H- 1,2,4-triazol-1-yl) pyridin-3-yl)-4-amino- pyrrolo[2,1-f][1,2,4] triazin-5-yl)piperidin- 3-yl)-6-(4-methyl- piperazin-1-yl) picolinamide | 580 | B: 1.06 | (500 MHz, DMSO-d₆) δ 9.40 (s, 1H), 9.22 (s, 1H), 8.78 (br dd, J = 8.5, 2.1 Hz, 1H), 8.33 (s, 1H), 8.29- 8.19 (m, 1H), 7.96 (br d, J = 8.5 Hz, 1H), 7.92 (s, 1H), 7.69 (br t, J = 7.9 Hz, 1H), 7.31 (br d, J = 7.3 Hz, 1H), 7.26 (s, 1H), 7.04 (br d, J = 7.9 Hz, 1H), 4.22 (ddd, J = 10.5, 4.3, 2.0 Hz, 1H), 3.43 (br s, 4H), 3.27-3.19 (m, 1H), 3.11-3.01 (m, 1H), 2.96- 2.77 (m, 2H), 2.46 (br d, J = 4.0 Hz, 4H), 2.26 (br s, 2H), 1.97-1.80 (m, 3H), 1.76-1.62 (m, 1H) |
| 801 | 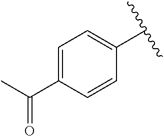 [SMILES: *c1ccc(C(C)=O)cc1] | (R)-N-(1-(7-(4- acetylphenyl)-4-amino- pyrrolo[2,1-f][1,2,4] triazin-5-yl)piperidin- 3-yl)-6-(4-methyl- piperazin-1-yl) picolinamide | 554 | B: 1.12 | (500 MHz, DMSO-d₆) δ 8.31-8.21 (m, 3H), 8.04 (d, J = 8.5 Hz, 2H), 7.99 (s, 1H), 7.78 (t, J = 7.9 Hz, 1H), 7.41 (d, J = 7.3 Hz, 1H), 7.23 (s, 1H), 7.16 (br d, J = 8.5 Hz, 1H), 4.32-4.18 (m, 1H), 3.24 (br d, J = 8.9 Hz, 1H), 3.15-3.07 (m, 1H), 2.87 (s, 4H), 2.85-2.74 (m, 1H), 2.61 (s, 3H), 2.55 (s, 3H), 2.55-2.54 (m, 1H), 2.51 (br s, 4H), 1.99-1.83 (m, 3H), 1.77-1.62 (m, 1H) |
| 802 | 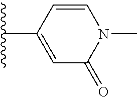 [SMILES: *c1ccn(C)c(=O)c1] | (R)-N-(1-(4-amino-7- (1-methyl-2-oxo-1,2- dihydropyridin-4-yl) pyrrolo[2,1-f][1,2,4] triazin-5-yl)piperidin- 3-yl)-6-(4-methyl- piperazin-1-yl) | 543 | B: 1.00 | (500 MHz, DMSO-d₆) δ 7.96 (s, 1H), 7.78 (t, J = 7.9 Hz, 1H), 7.70 (d, J = 7.0 Hz, 1H), 7.46-7.38 (m, 2H), 7.25 (d, J = 2.7 Hz, 2H), 7.19-7.12 (m, 2H), 7.05 (s, 1H), 6.89 (dd, J = 7.0, 1.8 Hz, 1H), 4.30-4.15 (m, 1H), 3.44-3.36 (m, 4H), 3.25-3.12 (m, 2H), 3.09- 3.02 (m, 1H), 2.87 (s, 3H), 2.82-2.73 (m, 1H), 2.55 (s, 3H), 2.51 (br s, 4H), 1.95-1.82 (m, 3H), 1.74- |
| | | picolinamide | | | 1.62 (m, 1H) |
| 803 | 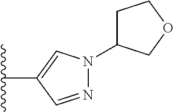 [SMILES: *c1cnn(C2CCOC2)c1] | N-((3R)-1-(4-amino- 7-(1-(tetrahydrofuran- 3-yl)-1H-pyrazol-4- yl)pyrrolo[2,1-f] [1,2,4]triazin-5-yl) piperidin-3-yl)-6-(4- methylpiperazin-1- yl)picolinamide | 572 | B: 1.07 | (500 MHz, DMSO-d₆) δ 8.40 (s, 1H), 8.24 (br d, J = 7.0 Hz, 1H), 8.08 (s, 1H), 7.84 (s, 1H), 7.70-7.66 (m, 1H), 7.31 (d, J = 7.0 Hz, 1H), 7.04 (d, J = 8.9 Hz, 1H), 6.90 (s, 1H), 5.17-5.07 (m, 1H), 5.07- 4.95 (m, 1H), 4.26-4.12 (m, 1H), 4.03-3.91 (m, 4H), 3.87-3.78 (m, 2H), 3.56 (br s, 4H), 3.24-3.13 (m, 1H), 3.02 (br dd, J = 10.5, 2.9 Hz, 1H), 2.93- 2.72 (m, 2H), 2.48-2.33 (m, 5H), 2.30-2.15 (m, |
| | | | | | 3H), 1.91-1.79 (m, 3H), 1.73-1.60 (m, 1H) |
| 804 | 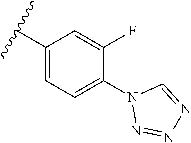 [SMILES: *c1ccc(-n2cnnn2)c(F)c1] | (R)-N-(1-(4-amino- 7-(3-fluoro-4-(1H- tetrazol-1-yl)phenyl) pyrrolo[2,1-f][1,2,4] triazin-5-yl)piperidin- 3-yl)-6-(4-methyl- piperazin-1-yl) picolinamide, TFA salt | 598 | B: 1.11 | (500 MHz, DMSO-d₆) δ 9.96 (d, J = 1.1 Hz, 1H), 8.46-8.37 (m, 1H), 8.29 (br d, J = 8.3 Hz, 1H), 8.25- 8.19 (m, 1H), 8.00-7.92 (m, 2H), 7.78 (t, J = 8.0 Hz, 1H), 7.41 (d, J = 7.2 Hz, 1H), 7.33 (s, 1H), 7.15 (d, J = 8.5 Hz, 1H), 4.29-4.17 (m, 1H), 3.44-3.39 (m, 4H), 3.23 (br d, J = 8.8 Hz, 1H), 3.16-3.05 (m, 4H), 2.87 (s, 3H), 2.83-2.74 (m, 1H), 1.95-1.83 (m, 3H), 1.76-1.62 (m, 1H) |
| 805 | 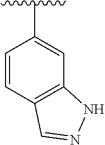 [SMILES: *c1ccc2cn[nH]c2c1] | (R)-N-(1-(4-amino- 7-(1H-indazol-6-yl) pyrrolo[2,1-f][1,2,4] triazin-5-yl)piperidin- 3-yl)-6-(4-methyl- piperazin-1-yl) picolinamide | 552 | B: 1.33 | (500 MHz, DMSO-d₆) δ 8.53 (s, 1H), 8.24 (br s, 1H), 8.07 (s, 1H), 7.90 (s, 1H), 7.80 (br d, J = 8.5 Hz, 1H), 7.72-7.65 (m, 2H), 7.31 (d, J = 7.0 Hz, 1H), 7.14 (s, 1H), 7.03 (br d, J = 8.5 Hz, 1H), 4.26- 4.17 (m, 1H), 3.56 (br s, 4H), 3.27-3.20 (m, 1H), 3.11-3.01 (m, 1H), 2.95-2.77 (m, 2H), 2.48-2.36 (m, 4H), 2.23 (s, 3H), 1.96-1.84 (m, 3H), 1.73-1.63 (m, 1H) |
| 806 | 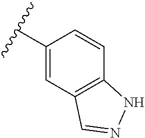 [SMILES: *c1ccc2[nH]ncc2c1] | (R)-N-(1-(4-amino- 7-(1H-indazol-5-yl) pyrrolo[2,1-f][1,2,4] triazin-5-yl)piperidin- 3-yl)-6-(4-methyl- piperazin-1-yl) picolinamide | 552 | B: 0.99 | (500 MHz, DMSO-d₆) δ 8.53 (s, 1H), 8.28 (br d, J = 8.9 Hz, 1H), 8.17 (s, 1H), 7.98 (s, 1H), 7.97-7.93 (m, 1H), 7.78 (t, J = 7.9 Hz, 1H), 7.63 (d, J = 8.9 Hz, 1H), 7.42 (d, J = 7.6 Hz, 1H), 7.17 (s, 1H), 7.08 (s, 1H), 4.64-4.47 (m, 1H), 4.34-4.21 (m, 1H), 3.91 (s, 4H), 3.30-3.23 (m, 1H), 3.02-3.09 (m, 1H), 3.00-2.77 (m, 4H), 2.51 (br s, 4H), 1.99- 1.83 (m, 3H), 1.76-1.61 (m, 1H) |
| 807 | 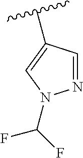 [SMILES: *c1cnn(C(F)F)c1] | (R)-N-(1-(4-amino-7- (1-(difluoromethyl)- 1H-pyrazol-4-yl) pyrrolo[2,1-f][1,2,4] triazin-5-yl)piperidin- 3-yl)-6-(4-methyl- piperazin-1-yl) picolinamide, TFA salt | 552 | B: 1.29 | (500 MHz, DMSO-d₆) δ 8.85 (s, 1H), 8.44 (s, 1H), 8.28 (br d, J = 8.3 Hz, 1H), 8.06-7.98 (m, 1H), 8.07- 7.73 (m, 2H), 7.41 (d, J = 7.2 Hz, 1H), 7.15 (br d, J = 8.5 Hz, 1H), 7.12 (s, 1H), 4.37-4.18 (m, 1H), 3.75 (br s, 4H), 3.22 (br d, J = 8.0 Hz, 1H), 3.13- 3.06 (m, 1H), 2.91-2.82 (m, 4H), 2.81-2.72 (m, 1H), 2.55 (s, 4H), 2.01-1.81 (m, 3H), 1.75-1.60 (m, 1H) |
| 808 | 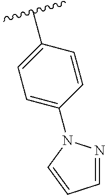 [SMILES: *c1ccc(-n2cccn2)cc1] | (R)-N-(1-(7-(4-(1H- pyrazol-1-yl)phenyl)- 4-aminopyrrolo [2,1-f][1,2,4]triazin- 5-yl)piperidin-3-yl)- 6-(4-methylpiperazin- 1-yl)picolinamide | 578 | B: 1.43 | (500 MHz, DMSO-d₆) δ 8.54 (d, J = 1.9 Hz, 1H), 8.23 (br d, J = 8.5 Hz, 3H), 7.92 (br d, J = 8.5 Hz, 2H), 7.88 (s, 1H), 7.77 (s, 1H), 7.69 (t, J = 8.0 Hz, 1H), 7.32 (d, J = 7.2 Hz, 1H), 7.10 (s, 1H), 7.04 (br d, J = 8.5 Hz, 1H), 6.57 (s, 1H), 4.28-4.15 (m, 1H), 3.57 (br s, 4H), 3.23 (br d, J = 8.5 Hz, 1H), 3.10-3.01 (m, 1H), 2.95-2.73 (m, 2H), 2.47 (br s, 4H), 2.26 (s, 3H), 1.96-1.81 (m, 3H), 1.75-1.60 (m, 1H) |
| 809 | 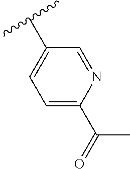 [SMILES: *c1ccc(C(C)=O)nc1] | (R)-N-(1-(7-(6- acetylpyridin-3-yl)-4- aminopyrrolo[2,1-f] [1,2,4]triazin-5-yl) piperidin-3-yl)-6-(4- methylpiperazin-1- yl)picolinamide | 555 | B: 1.19 | (500 MHz, DMSO-d₆) δ 9.40 (s, 1H), 8.74 (dd, J = 8.3, 1.6 Hz, 1H), 8.29-8.14 (m, 2H), 8.03 (d, J = 8.3 Hz, 1H), 7.94 (s, 1H), 7.68 (br t, J = 8.0 Hz, 1H), 7.34 (s, 1H), 7.30 (br d, J = 7.2 Hz, 1H), 7.03 (br d, J = 8.5 Hz, 1H), 4.24-4.18 (m, 1H), 3.55 (br s, 4H), 3.25-3.21 (m, 1H), 3.09-3.03 (m, 1H), 2.95-2.79 (m, 2H), 2.46-2.37 (m, 4H), 2.23 (s, 3H), 1.95-1.84 (m, 3H), 1.83 (s, 3H), 1.72-1.62 (m, 1H) |
| 810 | 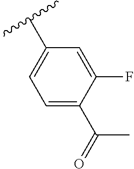 [SMILES: *c1ccc(C(C)=O)c(F)c1] | (R)-N-(1-(7-(4-acetyl- 3-fluorophenyl)-4- aminopyrrolo[2,1-f] [1,2,4]triazin-5-yl) piperidin-3-yl)-6-(4- methylpiperazin-1- yl)picolinamide | 572 | B: 1.31 | (500 MHz, DMSO-d₆) δ 8.23 (br d, J = 12.4 Hz, 2H), 8.09 (dd, J = 8.3, 1.4 Hz, 1H), 7.96 (s, 1H), 7.88 (t, J = 8.3 Hz, 1H), 7.68 (t, J = 7.8 Hz, 1H), 7.35-7.27 (m, 2H), 7.03 (d, J = 8.5 Hz, 1H), 4.27-4.15 (m, 1H), 3.56 (br s, 4H), 3.22 (br d, J = 8.0 Hz, 1H), 3.09- 3.00 (m, 1H), 2.93-2.73 (m, 2H), 2.60 (d, J = 4.1 Hz, 3H), 2.43 (br s, 4H), 2.24 (s, 3H), 1.94-1.80 (m, 3H), 1.69 (br d, J = 5.2 Hz, 1H) |
| 811 | 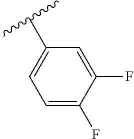 [SMILES: *c1ccc(F)c(F)c1] | (R)-N-(1-(4-amino-7- (3,4-difluorophenyl) pyrrolo[2,1-f][1,2,4] triazin-5-yl)piperidin- 3-yl)-6-(4- methylpiperazin-1- yl)picolinamide | 548 | B: 1.40 | (500 MHz, DMSO-d₆) δ 8.28 (br d, J = 8.0 Hz, 1H), 8.23-8.14 (m, 1H), 7.95 (s, 2H), 7.78 (br t, J = 7.9 Hz, 1H), 7.58-7.48 (m, 1H), 7.41 (br d, J = 7.4 Hz, 1H), 7.21-7.12 (m, 2H), 4.30-4.16 (m, 1H), 3.55 (br s, 4H), 3.21 (br d, J = 9.7 Hz, 1H), 3.11-3.02 (m, 2H), 2.86 (s, 4H), 2.81-2.75 (m, 1H), 2.51 (br s, 3H), 1.97-1.81 (m, 3H), 1.74-1.60 (m, 1H) |

### Example 812

### 4-methoxyphenyl (R)-(1-(7-(1-acetyl-2,5-dihydro-1H-pyrrol-3-yl)-4-aminopyrrolo[2,1-f][1,2,4]triazin-5-yl)piperidin-3-yl)carbamate TFA salt

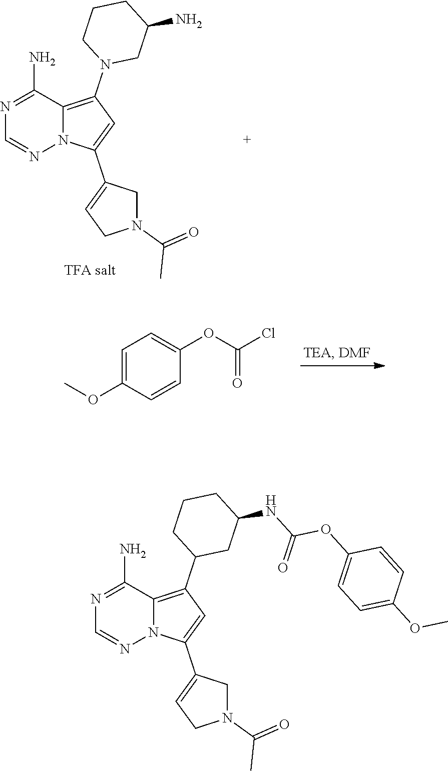 [SMILES: CC(=O)N1CC=C(c2cc(N3CCC[C@@H](N)C3)c3c(N)ncnn23)C1 ; COc1ccc(OC(=O)Cl)cc1 ; COc1ccc(OC(=O)N[C@@H]2CCCC(c3cc(C4=CCN(C(C)=O)C4)n4ncnc(N)c34)C2)cc1]

To a solution of Intermediate 32 (30 mg, 0.044 mmol) in DMF (1 mL) was added 4-methoxyphenyl chlorformate (9.8 mg, 0.053 mmol) followed by addition of TEA (0.061 mL, 0.44 mmol) at 0° C. The reaction was stirred under N₂ at 0° C. for 30 min. The reaction was quenched with H₂O. Purification by reverse phase chromatography provided Example 812 (15.7 mg, 55%). LCMS ESI m/z 492 (M+H)⁺, RT=1.34 min (Method B); ¹H NMR (500 MHz, DMSO-d₆) δ 8.04-7.91 (m, 2H), 7.03 (br d, J=8.9 Hz, 2H), 6.91 (br d, J=8.5 Hz, 2H), 6.84 (br s, 1H), 6.80 (br s, 1H), 4.71 (br s, 1H), 4.53 (br s, 1H), 4.49 (br s, 1H), 4.29 (br s, 1H), 3.83 (br d, J=1.8 Hz, 1H), 3.74 (s, 3H), 3.19-3.09 (m, 1H), 2.94-2.84 (m, 1H), 2.83-2.64 (m, 2H), 2.06 & 2.03 (s, 3H), 1.97-1.81 (m, 2H), 1.81-1.69 (m, 1H), 1.55-1.40 (m, 1H).

### Example 813

### (R)—N-(1-(7-(1-acetyl-2,5-dihydro-1H-pyrrol-3-yl)-4-aminopyrrolo[2,1-f][1,2,4]triazin-5-yl)piperidin-3-yl)-6-(piperazin-1-yl)picolinamide

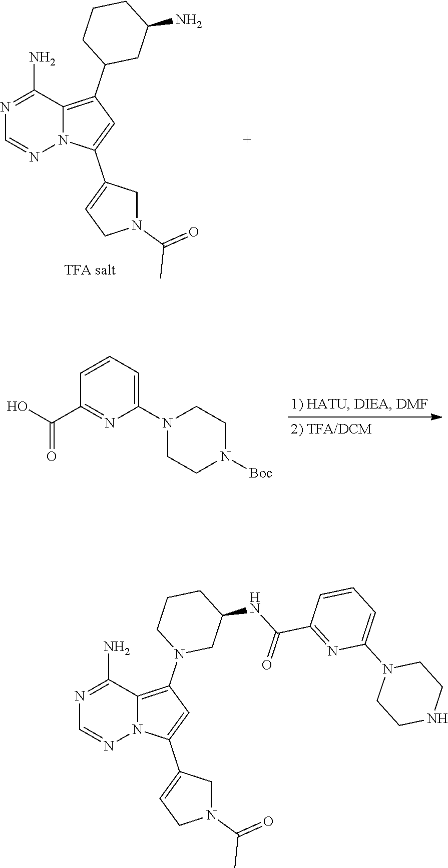 [SMILES: CC(=O)N1CC=C(c2cc(C3CCC[C@@H](N)C3)c3c(N)ncnn23)C1 ; CC(C)(C)OC(=O)N1CCN(c2cccc(C(=O)O)n2)CC1 ; CC(=O)N1CC=C(c2cc(N3CCC[C@@H](NC(=O)c4cccc(N5CCNCC5)n4)C3)c3c(N)ncnn23)C1]

To a solution of Intermediate 32 (30 mg, 0.044 mmol) in DMF (1 mL) was added Intermediate 54 (13.49 mg, 0.044 mmol) followed by addition of DIEA (0.077 mL, 0.439 mmol) and HATU (33.4 mg, 0.088 mmol) at 0° C. The reaction was stirred under N₂ at 0° C. for 1 h. The crude product was purified by reverse phase chromatography. The obtained product was dissolved in DCM (1 mL), and TFA (0.5 mL) was added at rt. The reaction was stirred at it for 1 h. Reverse phase chromatography provided Example 813 (5.9 mg, 35%). Analytical LCMS ESI m/z 531 (M+H)⁺, RT=0.95 min (Method B); ¹H NMR (500 MHz, DMSO-d₆) δ 8.21 (br s, 1H), 7.87 (d, J=1.8 Hz, 1H), 7.67 (br t, J=7.9 Hz, 1H), 7.28 (d, J=7.0 Hz, 1H), 7.00 (br d, J=8.9 Hz, 1H), 6.77 (br d, J=12.5 Hz, 1H), 6.72 (d, J=12.5 Hz, 1H), 4.69 (br s, 1H), 4.50 (br s, 1H), 4.47 (br s, 1H), 4.27 (br s, 1H), 4.21-4.12 (m, 1H), 3.51-2.93 (m, 8H), 2.85-2.75 (br s, 4H), 2.05, 2.01 (s, 3H), 1.82 (br s, 3H), 1.72-1.57 (m, 1H).

### Example 814

### N—((R)-1-(7-(1-acetyl-2,5-dihydro-1H-pyrrol-3-yl)-4-aminopyrrolo[2,1-f][1,2,4]triazin-5-yl)piperidin-3-yl)-6-((1R,5S)-3,8-diazabicyclo[3.2.1]octan-3-yl)picolinamide

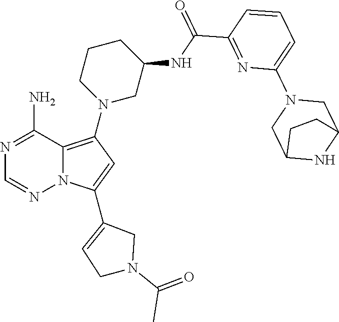 [SMILES: CC(=O)N1CC=C(c2cc(N3CCC[C@@H](NC(=O)c4cccc(N5CC6CCC(C5)N6)n4)C3)c3c(N)ncnn23)C1]

Example 814 was prepared by following a similar procedure as that described in Example 813 by using intermediate 55. LCMS ESI m/z 557 (M+H)⁺, RT=1.08 min (Method B); ¹H NMR (500 MHz, DMSO-d₆) δ 9.08 (br d, J=3.7 Hz, 1H), 9.02 (br s, 1H), 8.20 (br d, J=7.9 Hz, 1H), 7.98 (s, 1H), 7.76 (t, J=7.9 Hz, 1H), 7.39 (d, J=7.3 Hz, 1H), 7.04 (br d, J=8.5 Hz, 1H), 6.84-6.75 (m, 2H), 4.70 (br s, 1H), 4.51 (br s, 1H), 4.49 (br s, 1H), 4.32-4.14 (m, 6H), 3.05 br d, J=7.6 Hz, 1H), 2.98-2.89 (m, 1H), 2.86-2.70 (m, 2H), 2.03 (br d, J=18.9 Hz, 3H), 2.01-1.83 (m, 7H), 1.70-1.56 (m, 1H).

### Example 815

### (R)—N-(1-(7-(1-acetyl-2,5-dihydro-1H-pyrrol-3-yl)-4-aminopyrrolo[2,1-f][1,2,4]triazin-5-yl)piperidin-3-yl)-4,5,6,7-tetrahydrothieno[3,2-c]pyridine-2-carboxamide TFA salt

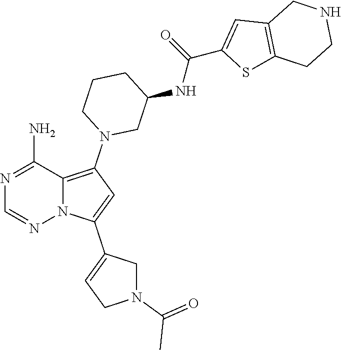 [SMILES: CC(=O)N1CC=C(c2cc(N3CCC[C@@H](NC(=O)c4cc5c(s4)CCNC5)C3)c3c(N)ncnn23)C1]

Example 815 was prepared by following a similar procedure as described in Example 813 by using 5-(tert-butoxycarbonyl)-4,5,6,7-tetrahydrothieno[3,2-c]pyridine-2-carboxylic acid. LCMS ESI m/z 507 (M+H)⁺, RT=0.94 min (Method B); ¹H NMR (500 MHz, DMSO-d₆) δ 9.17 (br s, 2H), 8.39 (dd, J=7.7, 1.7 Hz, 1H), 7.97 (d, J=1.7 Hz, 1H), 7.60 (s, 1H), 6.86-6.75 (m, 2H), 4.70 (br s, 1H), 4.51 (br d, J=2.5 Hz, 1H), 4.48 (br d, J=1.8 Hz, 1H), 4.28 (br d, J=2.4 Hz, 1H), 4.21 (br s, 2H), 4.17-4.08 (m, 1H), 3.44 (br d, J=4.7 Hz, 2H), 3.23-3.18 (m, 1H), 3.06 (br t, J=5.9 Hz, 3H), 2.75-2.59 (m, 2H), 2.03 (d, J=19.6 Hz, 3H), 1.94-1.79 (m, 3H), 1.58-1.45 (m, 1H).

The following examples in Table 57 were prepared using a similar procedure as described in Example 785 by coupling Intermediate 63 with catalysts other than PdCl₂(dppf) and solvents, such as dioxane/H₂O, THF/H₂O, or DMF/H₂O were alternatively used.

TABLE 57

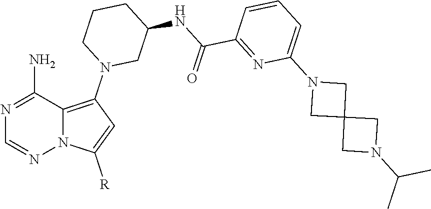

| Example | R | Name | LCMS (M + H)⁺ | HPLC Method, RT (min.) | ¹H NMR |
|---|---|---|---|---|---|
| 816 | 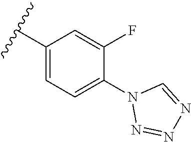 [SMILES: *c1ccc(-n2cnnn2)c(F)c1] | (R)-N-(1-(4-amino-7-(3- fluoro-4-(1H-tetrazol-1- yl)phenyl)pyrrolo[2,1- f][1,2,4]triazin-5-yl)piperidin- 3-yl)-6-(6-isopropyl-2,6- diazaspiro[3.3]heptan-2- yl)picolinamide | 638 | 1.28 | (500 MHz, DMSO-d₆) δ 9.97 (s, 1H), 8.44 (br d, J = 12.9 Hz, 1H), 8.25 (br d, J = 8.3 Hz, 1H), 8.00-7.91 (m, 2H), 7.67 (br t, J = 7.8 Hz, 1H), 7.35 (s, 1H), 7.29 (br d, J = 7.3 Hz, 1H), 6.59 (br d, J = 7.9 Hz, 1H), 4.27-4.16 (m, 1H), 4.08 (br s, 4H), 3.30-3.18 (m, 1H), 3.12-3.03 (m, 1H), 3.00 (s, 4H), 2.93-2.77 (m, 2H), 2.47-2.34 (m, 1H), 2.00-1.79 (m, 3H), 1.72-1.57 (m, 1H), 0.89 (br d, J = 5.4 Hz, 6H) |
| 817 | 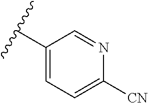 [SMILES: *c1ccc(C#N)nc1] | (R)-N-(1-(4-amino-7-(6- cyanopyridin-3- yl)pyrrolo[2,1- f][1,2,4]triazin-5-yl)piperidin- 3-yl)-6-(6-isopropyl-2,6- diazaspiro[3.3]heptan-2- yl)picolinamide | 578 | 1.35 | (500 MHz, DMSO-d₆) δ 9.44 (d, J = 1.5 Hz, 1H), 8.81 (dd, J = 8.2, 1.8 Hz, IH), 8.24 (br s, 2H), 8.09 (d, J = 8.2 Hz, 1H), 7.96 (s, 1H), 7.66 (t, J = 7.8 Hz, 1H), 7.43 (s, 1H), 7.28 (d, J = 7.0 Hz, 1H), 6.94 (br s, 1H), 6.58 (d, J = 8.2 Hz, 1H), 4.28-4.14 (m, 1H), 4.06 (s, 4H), 3.32-3.16 (m, 1H), 3.11-2.93 (m, 2H), 2.90-2.71 (m, 1H), 2.32-2.18 (m, 1H), 1.96-1.79 (m, 3H), 1.74-1.54 (m, 1H), 0.85 (br d, J = 6.1 Hz, 6H) |
| 818 | 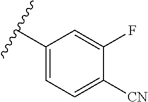 [SMILES: *c1ccc(C#N)c(F)c1] | (R)-N-(1-(4-amino-7-(4- cyano-3- fluorophenyl)pyrrolo[2,1- f][1,2,4]triazin-5-yl)piperidin- 3-yl)-6-(6-isopropyl-2,6- diazaspiro[3.3]heptan-2- yl)picolinamide | 595 | 1.35 | (500 MHz, DMSO-d₆) δ 8.36 (br d, J = 11.3 Hz, 1H), 8.21 (br d, J = 8.5 Hz, 1H), 7.99-7.92 (m, 2H), 7.66 (t, J = 7.8 Hz, 1H), 7.39 (s, 1H), 7.28 (d, J = 7.3 Hz, 1H), 6.57 (d, J = 8.2 Hz, 1H), 4.25-4.14 (m, 1H), 4.11-3.99 (m, 4H), 3.22-3.16 (m, 1H), 3.09-2.96 (m, 2H), 2.93-2.71 (m, 1H), 2.26-2.12 (m, 1H), 1.98-1.79 (m, 3H), 1.70-1.55 (m, 1H), 0.84 (d, J = 6.1 Hz, 6H) |
| 819 | 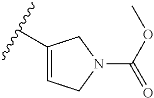 [SMILES: *C1=CCN(C(=O)OC)C1] | methyl (R)-3-(4-amino-5-(3- (6-(6-isopropyl-2,6- diazaspiro[3.3]heptan-2- yl)picolinamido)piperidin-1- yl)pyrrolo[2,1- f][1,2,4]triazin-7-yl)-2,5- | 601 | 1.31 | (500 MHz, DMSO-d₆) δ 8.23 (br s, 1H), 7.86 (s, 1H), 7.66 (t, J = 7.8 Hz, 1H), 7.28 (d, J = 7.2 Hz, IH), 6.79-6.70 (m, 2H), 6.57 (d. J = 8.5 Hz, IH), 4.61-4.47 (m, 2H), 4.35-4.24 (m, 2H), 4.21- 4.11 (m, 1H), 4.05 (s, 4H), 3.66 and 3.65 (s, 3H), 3.23 (s, 4H), 3.15 (br d, J = 8.0 Hz, 1H), 3.02-2.93 (m, 1H), 2.86-2.71 (m, 2H), 2.26- 2.15 (m, 1H), 1.95-1.82 (m, 3H), 1.66-1.52 (m, 1H), 0.84 (d, |
| | | dihydro-1H-pyrrole-1- | | | J = 6.3 Hz, 6H) |
| | | carboxylate | | | |
| 820 | 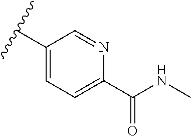 [SMILES: *c1ccc(C(=O)NC)nc1] | (R)-5-(4-amino-5-(3-(6-(6- isopropyl-2,6- diazaspiro[3.3]heptan-2- yl)picolinamido)piperidin-1- yl)pyrrolo[2,1- f][1,2,4]triazin-7-y])-N- methylpicolinamide | 610 | B: 1.23 | (500 MHz, DMSO-d₆) δ 9.30 (br s, 1H), 8.75 (br d, J = 7.7 Hz, 1H), 8.70 (br d, J = 8.0 Hz, 1H), 8.12-8.05 (m, 1H), 7.95 (br s, 1H), 7.74-7.66 (m, 1H), 7.39-7.24 (m, 2H), 6.63 (br d, J = 6.9 Hz, 1H), 4.27-4.19 (m, 1H), 4.13 (br s, 4H), 3.22-3.16 (m, 1H), 3.09- 3.01 (m, 1H), 2.95-2.65 (m, 5H), 1.98-1.81 (m, 3H), 1.69-1.57 (m, 1H), 1.17-0.79 (m, 6H) |
| 821 | 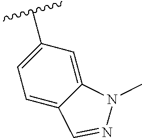 [SMILES: *c1ccc2cnn(C)c2c1] | (R)-N-(1-(4-amino-7-(1- methyl-1H-indazol-6- yl)pyrrolo[2,1- f][1,2,4]triazin-5-yl)piperidin- 3-yl)-6-(6-isopropyl-2,6- diazaspiro[3.3]heptan-2- yl)picolinamide | 606 | B: 1.22 | (500 MHz, DMSO-d₆) δ 8.34 (s, 1H), 8.04 (s, 1H), 7.89 (s, 1H), 7.87 (br d, J = 8.5 Hz, IH), 7.80 (d, J = 8.0 Hz, 1H), 7.67 (t, J = 7.8 Hz, 1H), 7.29 (d, J = 7.2 Hz, 1H), 7.17 (s, 1H), 6.58 (d, J = 8.3 Hz, 1H), 4.24-4.17 (m. 1H), 4.07 (d, J = 8.0 Hz, 7H), 3.28 (br s, 4H), 3.23 (br s, IH), 3.10-3.02 (m, 1H), 2.96-2.80 (m, 2H), 2.33- 2.19 (m, 1H), 1.93-1.80 (m, 3H), 1.71-1.58 (m, 1H), 0.86 (br d, J = 6.1 Hz, 6H) |
| 822 | 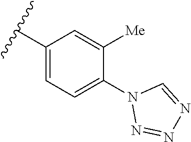 [SMILES: *c1ccc(-n2cnnn2)c(C)c1] | (R)-N-(1-(4-amino-7-(3- methyl-4-(1H-tetrazol-1- yl)phenyl)pyrrolo[2,1- f\|[1,2,4]triazin-5-yl)piperidin- 3-yl)-6-(6-isopropyl-2,6- diazaspiro[3.3]heptan-2- yl)picolinamide | 634 | B: 1.44 | (500 MHz, DMSO-d₆) δ 9.87 (s, 1H), 8.23-8.16 (m, 2H), 8.03- 7.92 (m, 2H), 7.71 (t, J = 7.8 Hz, 1H), 7.63 (d, J = 8.2 Hz, 1H), 7.33 (br d, J = 7.2 Hz, 1H), 7.24 (s, 1H), 6.65 (br d, J = 8.5 Hz, 1H), 4.35- 4.19 (m, 7H), 4.18-4.09 (m, 2H), 3.64-3.60 (m, 1H), 3.43-3.38 (m, 1H), 3.31-3.22 (m, 1H), 3.13-3.05 (m, 1H), 2.87 (br d, J = 7.0 Hz, 1H), 2.22 (s, 3H), 1.99-1.82 (m, 3H), 1.70-1.57 (m, 1H), 1.13 (br d, J = 6.1 Hz, 6H) |
| 823 | 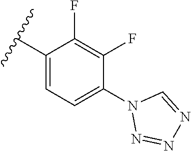 [SMILES: *c1ccc(-n2cnnn2)c(F)c1F] | (R)-N-(1-(4-amino-7-(2,3- difluoro-4-(1H-tetrazol-1- yl)phenyl)pyrrolo[2,1- f][1,2,4]triazin-5-yl)piperidin- 3-yl)-6-(6-isopropyl-2,6- diazaspiro[3.3]heptan-2- yl)picolinamide | 656 | B: 1.31 | (500 MHz, DMSO-d₆) δ 10.02 (s, 1H), 8.13 (br t, J = 7.0 Hz, 1H), 7.88 (s, 1H), 7.85 (brt, J = 7.9 Hz, 1H), 7.66 (t, J = 7.8 Hz, 1H), 7.33- 7.26 (m, 1H), 7.04 (s, 1H), 6.58 (br d, J = 8.5 Hz, 1H), 4.20 (br d, J = 6.7 Hz, 1H), 4.07 (s, 4H), 3.34 (br s, 2H), 3.21 (br d, J = 11.3 Hz, 1H), 3.04 (br s, 1H), 3.00 (s, 2H), 2.96-2.76 (m, 2H), 2.33 (br d, J = 3.7 Hz, 1H), 1.95-1.81 (m, 3H), 1.61 (br d, J = 14.0 Hz, 1H), 0.87 (br d, J = 6.1 Hz, 6H) |
| 824 | 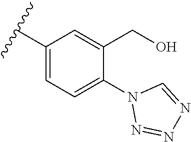 [SMILES: *c1ccc(-n2cnnn2)c(CO)c1] | (R)-N-(1-(4-amino-7-(3- (hydroxymethyl)-4-(1H- tetrazol-1- yl)phenyl)pyrrolo[2,1- f][1,2,4]triazin-5-yl)piperidin- 3-yl)-6-(6-isopropyl-2,6- diazaspiro[3.3]beptan-2- yl)picolinamide | 650 | B: 1.09 | (500 MHz, DMSO-d₆) δ 9.83 (s, 1H), 8.39 (s, 1H), 8.32-8.26 (m, 1H), 7.91 (s, 1H), 7.74-7.61 (m, 2H), 7.30 (d, J = 7.3 Hz, 1H), 7.20 (s, 1H), 6.60 (d, J = 8.5 Hz, 1H), 4.44 (s, 2H), 4.24-4.18 (m, 1H), 4.13-4.06 (m, 4H), 3.52-3.50 (m, 4H), 3.25-3.24 (m, 1H), 3.07- 3.02 (m, 1H), 2.96-2.87 (m, 2H), 1,92-1.82 (m, 4H), 1.67- 1.59 (m, 1H), 0.92 (br d, J = 6.1 Hz, 6H) |
| 825 | 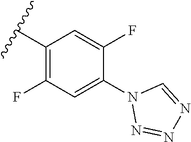 [SMILES: *c1cc(F)c(-n2cnnn2)cc1F] | (R)-N-(1-(4-amino-7-(2,5- difluoro-4-(1H-tetrazol-1- yl)phenyl)pyrrolo[2,1- f][1,2,4]triazin-5-yl)piperidin- 3-yl)-6-(6-isopropyl-2,6- diazaspiro[3.3]heptan-2- yl)picolinamide | 656 | B: 1.27 | (500 MHz, DMSO-d₆) δ 10.01 (s, 1H), 8.45 (br dd, J = 11.6, 6.1 Hz, 1H), 8.07 (br dd, J = 10.1, 6.1 Hz, 1H), 7.92 (s, IH), 7.67 (t, J = 7.9 Hz, 1H), 7.29 (d, J = 7.3 Hz, 1H), 7.07 (d, J = 2.4 Hz, 1H), 6.58 (d, J = 8.2 Hz, 1H), 4.24-4.18 (m, 1H), 4.07 (s, 4H), 3.27 (br s, 4H), 3.22 (br d, J = 1.2 Hz, 1H), 3.08-3.02 (m, 1H), 2.90-2.81 (m, 2H), 2.30-2.21 (m, 1H), 1.91-1.81 (m, 3H), 1.67-1.58 (m, 1H), 0.85 (d, J = 6.1 Hz, 6H) |
| 826 | 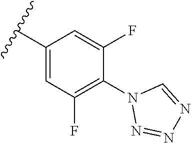 [SMILES: *c1cc(F)c(-n2cnnn2)c(F)c1] | (R)-N-(1-(4-amino-7-(3,5- difluoro-4-(1H-tetrazol-1- yl)phenyl)pyrrolo[2,1- f][1,2,4]triazin-5-yl)piperidin- 3-yl)-6-(6-isopropyl-2,6- diazaspiro[3.3]heptan-2- yl)picolinamide | 656 | B: 1.31 | (500 MHz, DMSO-d₆) δ 10.01 (s, 1H), 8.36 (br d, J = 10.4 Hz, 2H), 8.00 (s, 1H), 7.66 (t, J = 7.8 Hz, 1H), 7.47 (s, 1H), 7.28 (d, J = 7.3 Hz, 1H), 6.57 (d, J = 8.2 Hz, 1H), 4.20 (br d, J = 5.2 Hz, 1H), 4.11- 3.99 (m, 4H), 3.33-3.12 (m, 5H), 3.05 (br dd, J = 6.9, 2.9 Hz, 1H), 2.95-2.72 (m, 2H), 2.21 (dt, J = 12.2, 6.1 Hz, 1H), 1.99-1.81 (m, 3H), 1.72-1.53 (m, 1H), 0.84 (d, J = 6.1 Hz, 6H) |
| 827 | 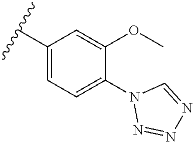 [SMILES: *c1ccc(-n2cnnn2)c(OC)c1] | (R)-N-(1-(4-amino-7-(3- methoxy-4-(1H-tetrazol-1- yl)phenyl)pyrrolo[2,1- f][1,2,4]triazin-5-y1)piperidin- 3-yl)-6-(6-isopropyl-2,6- diazaspiro[3.3]heptan-2- yl)picolinamide | 650 | B: 1.23 | (500 MHz, DMSO-d₆) δ 9.82 (s, 1H), 8.04-7.88 (m, 3H), 7.76 (d, J = 8.2 Hz, 1H), 7.67 (t, J = 7.8 Hz, 1H), 7.33-7.25 (m, 2H), 6.58 (d, J = 8.2 Hz, 1H), 4.24-4.17 (m, 1H), 4.11-4.02 (m, 4H), 3.96 (s, 3H), 3.29 (s, 4H), 3.25-3.21 (m, 1H), 3.08-3.02 (m, 1H), 2.94- 2.82 (m, 2H), 2.34-2.21 (m, 1H), 1.86 (br s, 3H), 1.70-1.57 (m, 1H), 0.86 (d, J = 6.1 Hz, 6H) |
| 828 | 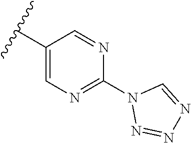 [SMILES: *c1cnc(-n2cnnn2)nc1] | (R)-N-(1-(7-(2-(1H-tetrazol- 1-yl)pyrimidin-5-yl)-4- aminopyrrolo[2,1- f][1,2,4]triazin-5-yl)piperidin- 3-yl)-6-(6-isopropyl-2,6- diazaspiro[3.3]heptan-2- yl)picolinamide | 622 | B: 1.31 | (500 MHz, DMSO-d₆) δ 10.25 (s, 1H), 9.70 (s, 2H), 7.98 (s, 1H), 7.67 (t, J = 7.8 Hz, 1H), 7.45 (s, 1H), 7.29 (d, J = 7.2 Hz, 1H), 6.59 (d. J = 8.4 Hz, 1H), 4.22 (br dd, J = 7.7, 3.7 Hz, 1H), 4.13-4.01 (m, 4H), 3.40 (br s, 4H), 3.25-3.23 (m, 1H), 3.09-3.03 (m, 1H), 2.92- 2.81 (m, 2H), 2.42-2.33 (m, 1H), 1.92-1.84 (m, 3H), 1.69- 1.59 (m, 1H), 0.88 (d, J = 6.2 Hz, 6H) |
| 829 | 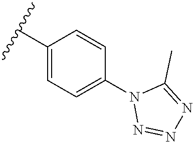 [SMILES: *c1ccc(-n2nnnc2C)cc1] | (R)-N-(1-(4-amino-7-(4-(5- methyl-1H-tetrazol-1- yl)phenyl)pyrrolo[2,1- f][1,2,4]triazin-5-yl)piperidin- 3-yl)-6-(6-isopropyl-2,6- diazaspiro[3.3]heptan-2- yl)picolinamide | 634 | B: 1.19 | (500 MHz, DMSO-d₆) δ 8.37 (d, J = 8.5 Hz, 2H), 7.91 (s, IH), 7.76 (d, J = 8.5 Hz, 2H), 7.67 (t, J = 7.8 Hz, 1H), 7.29 (d, J = 7.0 Hz, 1H), 7.22 (s, 1H), 6.58 (d, J = 8.2 Hz, 1H), 4.25-4.16 (m, 1H), 4.06 (s, 4H), 3.25 (s, 4H), 3.23-3.17 (m, 1H), 3.06 (br d, J = 9.2 Hz, 1H), 2.97-2.76 (m, 2H), 2.61 (s, 3H), 2.28-2.16 (m, 1H), 1.97-1.81 (m, 3H), 1.72-1.56 (m, 1H), 0.84 (d, J = 6.1 Hz, 6H) |
| 830 | 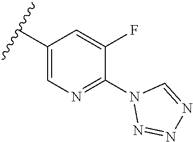 [SMILES: *c1cnc(-n2cnnn2)c(F)c1] | (R)-N-(1-(4-amino-7-(5- fluoro-6-(1H-tetrazol-1- yl)pyridin-3-yl)pyrrolo[2,1- f][1,2,4]triazin-5-yl)piperidin- 3-yl)-6-(6-isopropyl-2,6- diazaspiro[3.3]heptan-2- yl)picolinamide | 639 | B: 1.20 | (500 MHz, DMSO-d₆) δ 10.11 (s, 1H), 9.25 (s, 1H), 8.93 (dd, J = 12.1, 1.4 Hz, 1H), 8.25 (br s, 2H), 7.99 (s, 1H), 7.67 (t, J =7.8 Hz, 1H), 7.48 (s, 1H), 7.29 (d, J = 7.3 Hz, 1H), 6.94 (br s, 1H), 6.58 (d. J = 8.2 Hz, 1H), 4.27-4.16 (m, 1H), 4.11-4.02 (m, 4H), 3.32- 3.27 (m, 4H), 3.23 (br s, 1H), 3.10-3.02 (m, 1H), 2.96-2.76 (m, 2H), 2.31-2.20 (m, 1H), 1.97-1.83 (m, 3H), 1.64 (td, J = 8.8, 5.3 Hz, 1H), 0.85 (d, J = 6.1 Hz, 6H) |
| 831 | 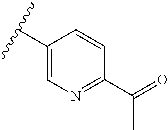 [SMILES: *c1ccc(C(C)=O)nc1] | (R)-N-(1-(7-(6-acetylpyridin- 3-yl)-4-aminopyrrolo[2,1- f][1,2,4]triazin-5-yl)piperidin- 3-yl)-6-(6-isopropyl-2,6- diazaspiro[3.3]heptan-2- yl)picolinamide | 595 | B: 1.22 | (500 MHz, DMSO-d₆) δ 9.40 (d, J = 1.8 Hz, 1H), 8.73 (dd, J = 8.4, 2.0 Hz, 1H), 8.35-8.15 (m, 1H), 8.06-7.92 (m, 1H), 7.70-7.63 (m, 1H), 7.37-7.24 (m, 2H), 6.58 (br d, J = 8.2 Hz, 1H), 4.28-4.13 (m, 1H), 4.06 (s, 4H), 3.32-3.20 (m, 1H), 3.12-2.99 (m, 1H), 2.98-2.67 (m, 2H), 2.55 (s, 3H), 2.26 (br dd, J = 11.9, 6.7 Hz, 1H), 1.86 (br s, 3H), 1.68-1.54 (m, 1H), 0.85 (br s, 6H). Four protons from diazaspiroheptane did not show due to water suppression |
| 832 | 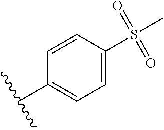 [SMILES: *c1ccc(S(C)(=O)=O)cc1] | (R)-N-(1-(4-amino-7-(4- (methylsulfonyl)phenyl)pyrro- lo[2,1-f][1,2,4]triazin-5- yl)piperidin-3-yl)-6-(6- isopropyl-2,6- diazaspiro[3.3]heptan-2- yl)picolinamide | 630 | B: 1.17 | (500 MHz, DMSO-d₆) δ 8.38 (d, J = 8.5 Hz, 2H), 7.97 (d, J = 8.5 Hz, 2H), 7.92 (s, 1H), 7.67 (t, J = 7.8 Hz, 1H), 7.29 (d, J = 7.3 Hz, 1H), 7.25 (s, 1H), 6.58 (d, J=8.5 Hz, IH), 4.24 - 4.17 (m, 1H), 4.06 (s, 4H), 3.24 (s, 7H), 3.22 (br s, 1H), 3.08-3.02 (m, 1H), 2.93-2.77 (m, 2H), 2.26-2.18 (m, 1H), 1.95-1.82 (m, 3H), 1.67-1.58 (m, 1H), 0.85 (d, J = 6.1 Hz, 6H) |
| 833 | 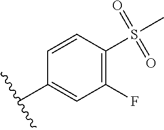 [SMILES: *c1ccc(S(C)(=O)=O)c(F)c1] | (R)-N-(1-(4-amino-7-(3- fluoro-4- (methylsulfonyl)phenyl)pyrro- lo[2,1-f][1,2,4]triazin-5- yl)piperidin-3-yl)-6-(6- isopropyl-2,6- diazaspiro[3.3]heptan-2- yl)picolinamide | 648 | B: 1.21 | (500 MHz, DMSO-d₆) δ 8.34 (br d, J = 12.5 Hz, 1H), 8.23-8.19 (m, 1H), 7.97 (s, 1H), 7.89 (t, J = 8.1 Hz, 1H), 7.67 (t, J = 7.8 Hz, 1H), 7.36 (s, 1H), 7.29 (d, J = 7.3 Hz, 1H), 6.59 (d, J = 8.2 Hz, 1H), 4.20 (br s, 1H), 4.12-4.05 (m, 4H), 3.49-3.48 (m, 4H), 3.35 (s, 3H), 3.22 (br d, J = 11.6 Hz, 1H), 3.08-3.00 (m, 1H), 2.93-2.76 (m, 2H), 2.47 (br s, 1H), 1.86 (br s, 3H), 1.69-1.58 (m, 1H), 0.91 (br d, J = 6.1 Hz, 6H) |
| 834 | 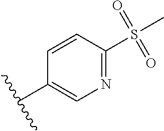 [SMILES: *c1ccc(S(C)(=O)=O)nc1] | (R)-N-(1-(4-amino-7-(6- (methylsulfonyl)pyridin-3- yl)pyrrolo[2,1- f][1,2,4]triazin-5-yl)piperidin- 3-yl)-6-(6-isopropyl-2,6- diazaspiro[3.3]heptan-2- yl)picolinamide | 631 | B: 1.12 | (500 MHz, DMSO-d₆) δ 9.42 (d, J = 1.5 Hz, 1H), 8.87 (dd, J = 8.4, 2.0 Hz, 1H), 8.12 (d, J = 8.2 Hz, 1H), 7.96 (s, 1H), 7.68 (t, J = 7.8 Hz, 1H), 7.39 (s, 1H), 7.30 (d, J = 7.0 Hz, 1H), 6.60 (d, J = 8.5 Hz, 1H), 4.26-4.18 (m, 1H), 4.13-4.08 (m, 4H), 3.60 (br s, 4H), 3.31 (s, 3H), 3.23 (br d, J = 8.2 Hz, 1H), 3.07-3.02 (m, 1H), 2.93-2.78 (m, 3H), 2.67-2.59 (m, 1H), 1.87 (br s, 3H), 1.69-1.58 (m, 1H), 0.94 (br d, J = 6.1 Hz, 6H) |

### Example 835

### (R)-5-(4-amino-5-(3-(5-chloro-3-(2-(dimethylamino)ethoxy)thiophene-2-carboxamido)piperidin-1-yl)pyrrolo[2,1-f][1,2,4]triazin-7-yl)-N-methylpicolinamide

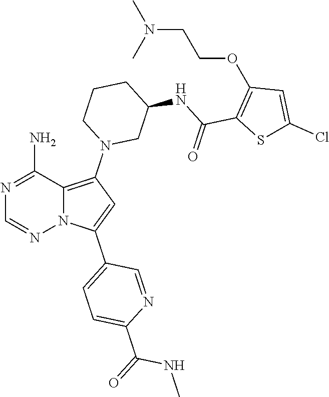 [SMILES: CNC(=O)c1ccc(-c2cc(N3CCC[C@@H](NC(=O)c4sc(Cl)cc4OCCN(C)C)C3)c3c(N)ncnn23)cn1]

Example 835 was prepared using a similar procedure as described in Example 785 by coupling Example 171A with N-methyl-5-(4,4,5,5-tetramethyl-1,3,2-dioxaborolan-2-yl)picolinamide. LC-MS Method B. RT=1.26 min, [M+H]⁺=598; ¹H NMR (500 MHz, DMSO-d₆) δ ppm (500 MHz, DMSO-d₆) δ 9.28 (d, J=1.4 Hz, 1H), 8.78-8.71 (m, 1H), 8.68 (dd, J=8.3, 2.0 Hz, 1H), 8.08 (d, J=8.5 Hz, 1H), 7.91 (s, 1H), 7.78-7.72 (m, 1H), 7.35 (s, 1H), 7.30 (s, 1H), 4.29 (br t, J=5.0 Hz, 2H), 4.17 (br d, J=2.0 Hz, 1H), 3.24-3.18 (m, 2H), 3.07-3.00 (m, 2H), 2.85 (d, J=4.7 Hz, 3H), 2.63-2.57 (m, 2H), 2.18 (br s, 6H), 1.95-1.71 (m, 4H)

The following Examples in Table 58 were prepared using a similar procedure as described in Example 785 by using Intermediate 60, and then reacted with the appropriate boronic acids or boronates, prepared as intermediates described above, or obtained from commercial sources. Different palladium catalysts other than PdCl₂(dppf) and solvents, such as dioxane/H₂O. THF/H₂O, or DMF/H₂O were alternatively used.

TABLE 58

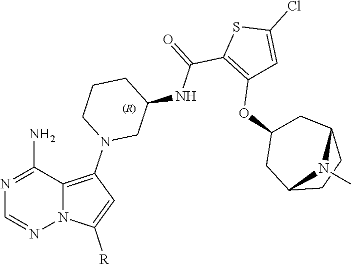

| Example | R | Name | LCMS (M + H)⁺ | HPLC Method, RT (min.) | ¹H NMR |
|---|---|---|---|---|---|
| 836 | 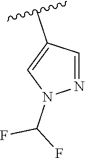 [SMILES: *c1cnn(C(F)F)c1] | N-((R)-1-(4-amino-7-(1- (difluoromethyl)-1H-pyrazol-4- yl)pyrrolo[2,1-f][1,2,4]triazin- 5-yl)piperidin-3-yl)-5-chloro-3- (((1R,3s,5S)-8-methyl-8- azabicyclo[3.2.1]octan-3- yl)oxy)thiophene-2- carboxamide | 632 | B: 1.33 | (500 MHz, DMSO-d₆) δ 8.83 (s, 1H), 8.41 (s, 1H), 7.97 (s, 1H), 7.90 (t, J = 58.5 Hz, 1H), 7.55 (s, 1H), 7.19 (br s, 1H), 7.11 (s. 1H), 4.96-4.79 (m, 1H), 4.23-4.12 (m, 1H), 3.96 (br s, 2H), 3.26-3.19 (m, 1H), 3.05 (br d, J = 3.0 Hz, 1H), 2.88-2.73 (m, 2H), 2.69 (br s, 3H), 2.43-2.32 (m, 2H), 2.26-2.11 (m, 4H), 2.09-2.00 (m, 2H), 1.99-1.86 (m, 2H), 1.85-1.76 (m, 1H), 1.59-1.44 (m, 1H) |
| 837 | 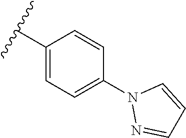 [SMILES: *c1ccc(-n2cccn2)cc1] | N-((R)-1-(7-(4-(1H-pyrazol-1- yl)phenyl)-4-aminopyrrolo[2,1- f][1,2,4]triazin-5-yl)piperidin- 3-yl)-5-chloro-3-(((1R,3s,5S)- 8-methyl-8- azabicyclo[3.2.1]octan-3- yl)oxy)thiophene-2- carboxamide | 658 | B: 1.45 | (500 MHz, DMSO-d₆) δ 8.56 (d, J = 2.5 Hz, 1H), 8.20 (d, J = 8.8 Hz, 2H), 7.97-7.89 (m, 3H), 7.78 (d, J = 1.4 Hz, 1H), 7.57 (s, 1H), 7.13 (s, 1H), 6.58 (d, J = 1.9 Hz, IH), 4.88 (td, J = 10.5, 5.5 Hz, 1H), 4.19 (br d, J = 5.0 Hz, 1H), 3.97 (br s, 2H), 3.23 (br dd, J = 5.1, 2.3 Hz, 1H), 3.12-3.04 (m, 1H), 2.91-2.76 (m, 2H), 2.70 (br s, 3H), 2.44-2.34 (m, 2H), 2.28-2.12 (m, 4H), 2.10- 2.01 (m, 2H), 1.99-1.87 (m, 2H), 1.85-1.76 (m, 1H), 1.59- 1.45 (m, 1H) |
| 838 | 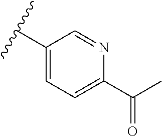 [SMILES: *c1ccc(C(C)=O)nc1] | N-((R)-1-(7-(6-acetylpyridin-3- yl)-4-aminopyrrolo[2,1- f][1,2,4]triazin-5-yl)piperidin- 3-yl)-5-chloro-3-(((1R,3s,5S)- 8-methyl-8- azabicyclo[3.2.1]octan-3- yl)oxy)thiophene-2- carboxamide | 635 | B: 1.35 | (500 MHz, DMSO-d₆) δ 9.35 (d, J = 1.7 Hz, 1H), 8.68 (dd, J = 8.4, 2.1 Hz, 1H), 7.98 (d, J = 8.3 Hz, 1H), 7.89 (s, 1H), 7.43 (s, 1H), 7.30 (s, 1H), 4.71-4.59 (m, 1H), 4.21-4.14 (m, 1H), 3.23- 3.12 (m, 2H), 3.10 (br s, 2H), 2.94-2.87 (m, 2H), 2.61 (s, 3H), 2.13 (s, 3H), 1.99-1.92 (m, 2H), 1.90-1.86 (m, 2H), 1.83- 1.74 (m, 3H), 1.70-1.61 (m, 4H), 1.58-1.45 (m, 1H) |
| 839 | 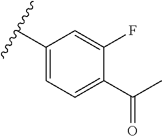 [SMILES: *c1ccc(C(C)=O)c(F)c1] | N-((R)-1-(7-(4-acetyl-3- fluorophenyl)-4- aminopyrrolo[2,1- f][1,2,4]triazin-5-yl)piperidin- 3-yl)-5-chloro-3-(((1R,3s,5S)- 8-methyl-8- azabicyclo[3.2.1]octan-3- yl)oxy)thiophene-2- carboxamide | 652 | B: 1.61 | (500 MHz, DMSO-d₆) δ 10.01-9.85 (m, 1H), 8.18 (d, J = 13.5 Hz, 1H), 8.06 (dd, J = 8.3, 1.4 Hz, 1H), 8.02 (s, 1H), 7.90 (t, J = 8.3 Hz, 1H), 7.56 (s, 1H), 7.34 (s, 1H), 7.22-7.15 (m, 1H), 4.94-4.82 (m, 1H), 4.23-4.13 (m, 1H), 3.97 (br s, 2H), 3.28- 3.20 (m, 1H), 3.12-3.05 (m, 1H), 2.91-2.77 (m, 2H), 2.70 (br d, J = 4.1 Hz, 4H), 2.61 (d, J = 3.9 Hz, 3H), 2.44-2.36 (m, 2H), 2.24-2.13 (m, 4H), 2.03 (br d, J = 12.9 Hz, 2H), 1.99-1.88 (m, 2H), 1.85-1.77 (m, 1H), 1.56-1.47 (m, 1H) |
| 840 | 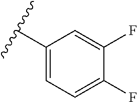 [SMILES: *c1ccc(F)c(F)c1] | N-((R)-1-(4-amino-7-(3,4- difluorophenyl)pyrrolo[2,1- f][1,2,4]triazin-5-yl)piperidin- 3-yl)-5-chloro-3-(((1R,3s,5S)- 8-methyl-8- azabicyclo[3.2.1]octan-3- yl)oxy)thiophene-2- | 628 | B: 1.51 | (500 MHz, DMSO-d₆) δ 8.23-8.14 (m, 1H), 7.96 (s, 2H), 7.61- 7.47 (m, 2H), 7.17 (s, 2H), 4.93-4.82 (m, 1H), 4.24-4.13 (m, 1H), 3.97 (br s, 2H), 3.27-3.18 (m, 1H), 3.12-3.03 (m, IH), 2.98-2.90 (m, 1H), 2.88-2.74 (m, 2H), 2.74-2.65 (m, 3H), 2.44-2.33 (m, 2H), 2.29-2.09 (m, 3H), 2.04 (q, J = 12.6 Hz, 2H), 1.99-1.86 (m, 2H), 1.84 -1.75 (m, 1H), 1.58-1.44 (m, 1H) |
| | | carboxamide | | | |
| 841 | 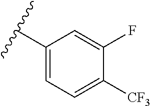 [SMILES: *c1ccc(C(F)(F)F)c(F)c1] | N-((R)-1-(4-amino-7-(3-fluoro- 4- (trifluoromethyl)phenyl)pyrrolo [2,1-f][1,2,4]triazin-5- yl)piperidin-3-yl)-5-chloro-3- (((1R,3s,5S)-8-methyl-8- azabicyclo[3.2.1]octan-3- | 678 | B: 1.80 | (500 MHz, DMSO-d₆) δ 8.31 (br d, J = 13.5 Hz, 1H), 8.18 (br d, J = 8.0 Hz, 1H), 7.95 (s, 1H), 7.83 (t, J = 8.3 Hz, 1H), 7.47 (s, 1H), 7.34 (s, 1H), 4.76-4.63 (m, 1H), 4.26-4.17 (m, 1H), 3.26- 3.17 (m, 2H), 3.13 (br s, 2H), 3.00-2.87 (m, 2H), 2.17 (s, 3H), 2.04-1.95 (m, 2H), 1.94-1.91 (m, 2H), 1.83 (br s, 3H), 1.74- 1.63 (m, 4H), 1.62-1.49 (m, 1H) |
| | | yl)oxy)thiophene-2- | | | |
| | | carboxamide | | | |
| 842 | 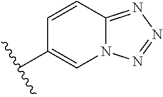 [SMILES: *c1ccc2nnnn2c1] | N-((R)-1-(4-amino-7- (tetrazolo[1,5-a]pyridin-6- yl)pyrrolo[2,1-f][1,2,4]triazin- 5-yl)piperidin-3-yl)-5-chloro-3- (((1R,3s,5S)-8-methyl-8- azabicyclo[3.2.1]octan-3- | 634 | B: 1.19 | (500 MHz, DMSO-d₆) δ 10.12 (s, IH), 8.49 (d. J = 9.5 Hz, 1H), 8.29 (d, J = 9.5 Hz, 1H), 8.03 (s, 1H), 7.57 (s, 1H), 7.18 (s, 1H), 4.95-4.84 (m, 1H), 4.23-4.14 (m, 1H), 3.97 (br s, 2H), 3.30- 3.14 (m, 1H), 3.13-3.03 (m, 1H), 2.98-2.76 (m, 2H), 2.70 (br d, J = 4.3 Hz, 3H), 2.39 (br d, J = 10.1 Hz, 2H), 2.25-2.04 (m, 6H), 1.99-1.87 (m, 2H), 1.85-1.77 (m, 1H), 1.62-1.46 (m, |
| | | yl)oxy)thiophene-2- | | | 1H) |
| | | carboxamide, TFA salt | | | |
| 843 | 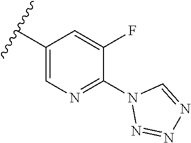 [SMILES: *c1cnc(-n2cnnn2)c(F)c1] | N-((R)-1-(4-amino-7-(5-fluoro- 6-(1H-tetrazol-1-yl)pyridin-3- yl)pyrrolo[2,1-f][1,2,4]triazin- 5-yl)piperidin-3-yl)-5-chloro-3- (((1R,3s,5S)-8-methyl-8- azabicyclo[3.2.1]octan-3- yl)oxy)thiophene-2- carboxamide | 679 | B: 1.32 | (500 MHz, DMSO-d₆) δ 10.10 (s, 1H), 9.24 (s, 1H), 8.93 (br d, J = 12.2 Hz, 1H), 7.99 (s, 1H), 7.48 (s, 2H), 4.79-4.67 (m, 1H), 4.27-4.16 (m, 1H), 3.26 (br s, 2H), 3.21-3.15 (m, 1H), 3.04- 2.77 (m, 3H), 2.25 (br s, 3H), 2.11-2.01 (m, 2H), 1.95 (br s, 2H), 1.91-1.81 (m, 3H), 1.80-1.68 (m, 4H), 1.65-1.53 (m, 1H) |

The following Examples in Table 59 were prepared using a similar procedure as described in Example 785 by using Intermediate 61 and Intermediate 66, and then reacted with the appropriate boronic acids or boronates, prepared as intermediates described above, or obtained from commercial sources. Palladium catalysts other than PdCl₂(dppf) and solvents, such as dioxane/H₂O, THF/H₂O, or DMF/H₂O can alternatively be used.

TABLE 59

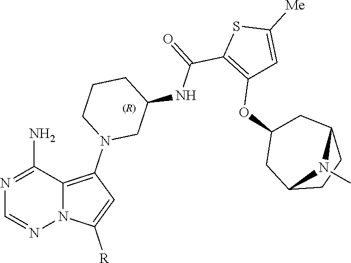

| Example | R | Name | LCMS (M + H)⁺ | HPLC Method, RT (min.) | ¹H NMR |
|---|---|---|---|---|---|
| 844 | 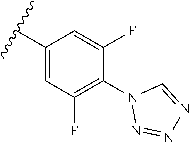 [SMILES: *c1cc(F)c(-n2cnnn2)c(F)c1] | N-((R)-1-(4-amino-7-(3,5- difluoro-4-(1H-tetrazol-1- yl)phenyl)pyrrolo[2,1- f][1,2,4]triazin-5-yl)piperidin- 3-yl)-5-methyl-3- (((1R,3s,5S)-8-methyl-8- azabicyclo[3.2.1]octan-3- yl)oxy)thiophene-2- carboxamide | 676 | B: 1.33 | (500 MHz, DMSO-d₆) δ 10.02 (s, IH), 8.37 (br d, J = 10.7 Hz, 2H), 8.01 (s, 1H), 7.48 (s, 1H), 7.02 (s, 1H), 4.68-4.59 (m, 1H), 4.25-4.18 (m, 1H), 3.16-3.13 (m, 2H), 3.02-2.78 (m, 4H), 2.42 (s, 3H), 2.18 (s, 3H), 2.01 (br d, J = 5.5 Hz, 2H), 1.96-1.91 (m, 2H), 1.87-1.79 (m, 3H), 1.71-1.63 (m, 4H), 1.60-1.49 (m, 1H) |
| 845 | 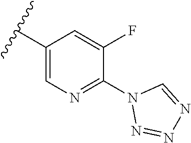 [SMILES: *c1cnc(-n2cnnn2)c(F)c1] | N-((R)-1-(4-amino-7-(5- fluoro-6-(1H-tetrazol-1- yl)pyridin-3-yl)pyrrolo[2,1- f][1,2,4]triazin-5-yl)piperidin- 3-yl)-5-methyl-3- (((1R,3s,5S)-8-methyl-8- azabicyclo[3.2.1]octan-3- yl)oxy)thiophene-2- carboxamide, TFA salt | 659 | B: 1.24 | (500 MHz, DMSO-d₆) δ 10.09 (s, 1H), 10.00 (br s, 1H), 9.22 (s, 1H), 8.95-8.86 (m, IH), 8.00 (s, 1H), 7.46 (s, 1H), 7.10 (s, 1H), 4.89-4.80 (m, 1H), 4.21-4.12 (m, 1H), 3.97 (br s, 2H), 3.29-3.22 (m, 1H), 3.11- 3.02 (m, 1H), 2.89-2.73 (m, 2H), 2.70 (br d, J = 3.4 Hz, 3H), 2.42 (s, 3H), 2.38 (br d, J = 8.9 Hz, 2H), 2.23 (br d, J = 4.3 Hz, 2H), 2.15-1.99 (m, 4H), 1.99-1.78 (m, 3H), 1.58-1.41 (m, 1H) |
| 846 | 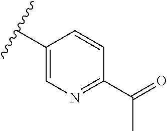 [SMILES: *c1ccc(C(C)=O)nc1] | N-((R)-1-(7-(6-acetylpyridin- 3-yl)-4-aminopyrrolo[2,1- f][1,2,4]triazin-5-yl)piperidin- 3-yl)-5-methyl-3- (((1R,3s,5S)-8-methyl-8- azabicyclo[3.2.1]octan-3- yl)oxy)thiophene-2- carboxamide | 615 | B: 1.25 | (500 MHz, DMSO-d₆) δ 9.40 (d, J = 1.2 Hz, 1H), 8.73 (dd, J = 8.5, 2.1 Hz, 1H), 8.03 (d, J = 8.2 Hz, IH), 7.94 (s, IH), 7.36 (s, 1H), 7.01 (s, 1H), 4.71-4.61 (m, 1H), 4.28-4.15 (m, IH), 3.16 (br d, J = 1.5 Hz, 1H), 3.07-2.84 (m, 3H), 2.66 (s, 3H), 2.42 (s, 3H), 2.19 (s, 3H), 2.07- 1.98 (m, 2H), 1.96-1.91 (m, 2H), 1.90-1.76 (m, 3H), 1.75-1.64 (m, 4H), 1.61-1.49 (m, 1H). |
| 847 | 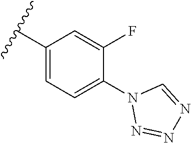 [SMILES: *c1ccc(-n2cnnn2)c(F)c1] | N-((R)-1-(4-amino-7-(3- fluoro-4-(1H-tetrazol-1- yl)phenyl)pyrrolo[2,1- f][1,2,4]triazin-5-yl)piperidin- 3-yl)-5-methyl-3- (((1R,3s,5S)-8-methyl-8- azabicyclo[3.2.1]octan-3- yl)oxy)thiophene-2- carboxamide, TFA salt | 658 | B: 1.28 | (500 MHz, DMSO-d₆) δ 9.98 (br s, 1H), 8.42 (br d, J = 12.5 Hz, 1H), 8.24 (br d, J = 8.2 Hz, 1H), 8.02-7.94 (m, 2H), 7.35 (br s, 1H), 7.12 (br s, 1H), 4.92-4.79 (m, 1H), 4.22-4.11 (m, 1H), 3.98 (br d, J = 2.1 Hz, 2H), 3.27-3.18 (m, 1H), 3.12-3.04 (m, 1H), 2.93-2.78 (m, 2H), 2.71 (br s, 3H), 2.43 (br s, 3H), 2.41-2.33 (m, 2H), 2.27-2.21 (m, 2H), 2.13 (td, J = 7.1, 1.7 Hz, 2H), 2.09-2.01 (m, 2H), 2.00-1.88 (m, 2H), 1.85-1.79 (m, 1H), 1.55-1.44 (m, 1H) |
| 848 | 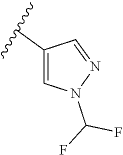 [SMILES: *c1cnn(C(F)F)c1] | N-((R)-1-(4-amino-7-(1- (difluoromethyl)- 1H-pyrazol- 4-yl)pyrrolo[2,1- f][1,2,4]triazin-5-yl)piperidin- 3-yl)-5-methyl-3- (((1R,3s,5S)-8-methyl-8- azabicyclo[3.2.1]octan-3- yl)oxy)thiophene-2- carboxamide, TFA salt | 612 | B: 1.25 | (500 MHz, DMSO-d₆) δ 10.02 (br s, 1H), 8.88-8.80 (m, 1H), 8.42 (s, 1H), 8.01 (s, 1H), 7.19-7.09 (m, 4H), 4.89-4.78 (m, 1H), 4.21-4.11 (m, 1H), 3.97 (br s, 2H), 3.30-3.25 (m, 1H), 3.12-3.05 (m, 1H), 2.84- 2.69 (m, 5H), 2.43 (br s, 3H), 2.40-2.33 (m, 2H), 2.23 (br d, J = 1.8 Hz, 2H), 2.17-2.10 (m, 2H), 2.07-1.92 (m, 4H), 1.86-1.76 (m, 1H), 1.52-1.42 (m, 1H) |

The following Examples in Table 60 were prepared using a similar procedure as described in Example 785 by using Intermediate 58 and Intermediate 61, and then reacted with the appropriate boronic acids or boronates, prepared as intermediates described above, or obtained from commercial sources. Palladium catalysts other than PdCl₂(dppf) and solvents, such as dioxane/H₂O, THF/1H₂O, or DMF/H₂O can alternatively be used

TABLE 60

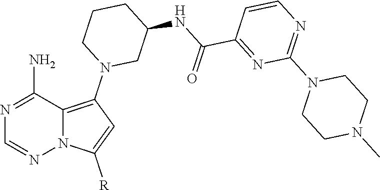

| Example | R | Name | LCMS (M + H)⁺ | HPLC Method, RT (min.) | ¹HNMR |
|---|---|---|---|---|---|
| 849 | 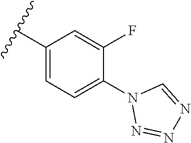 [SMILES: *c1ccc(-n2cnnn2)c(F)c1] | (R)-N-(1-(4-amino-7-(3- fluoro-4-(1H-tetrazol-1- yl)phenyl)pyrrolo[2,1- f][1,2,4]triazin-5- yl)piperidin-3-yl)-2-(4- methylpiperazin-1- yl)pyrimidine-4- carboxamide | 599 | B: 1.15 | (500 MHz, DMSO-d₆) δ 9.96 (s, 1H), 8.57 (d, J = 4.9 Hz, 1H), 8.51-8.41 (m, 2H), 8.25 (br d, J = 8.2 Hz, 1H), 8.00-7.93 (m, 2H), 7.33 (s, 1H), 7.11 (d, J = 4.9 Hz, 1H), 4.27-4.19 (m, 1H), 3.82 (br s, 4H), 3.21-3.17 (m, 1H), 3.10-3.03 (m, 1H), 2.96- 2.77 (m. 2H), 2.40 (br d. J = 4.3 Hz, 4H), 2.23 (s, 3H), 1.92-1.80 (m, 3H), 1.75-1.64 (m, 1H) |
| 850 | 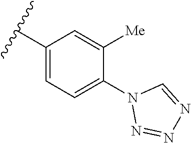 [SMILES: *c1ccc(-n2cnnn2)c(C)c1] | (R)-N-(1-(4-amino-7-(3- methyl-4-(1H-tetrazol-1- yl)phenyl)pyrrolo[2,1- f][1,2,4]triazin-5- yl)piperidin-3-yl)-2-(4- methylpiperazin-1- yl)pyrimidine-4- carboxamide | 595 | B: 1.14 | (500 MHz, DMSO-d₆) δ 9.87 (s, 1H), 8.57 (d, J = 4.9 Hz, 1H), 8.46 (br d, J = 8.2 Hz, 1H), 8.27-8.18 (m, 2H), 7.91 (s, 1H), 7.61 (d. J = 8.2 Hz, IH), 7.20 (s, 1H), 7.12 (d, J = 4.9 Hz, 1H), 4.27- 4.19 (m, 1H), 3.82 (br s, 4H), 3.23-3.18 (m, 1H), 3.11-3.04 (m, 1H), 2.96-2.88 (m, 1H), 2.84-2.76 (m, 1H), 2.39 (br t, J = 4.6 Hz, 4H), 2.24 (s, 3H), 2.21 (s, 3H), 1.96-1.84 (m, 3H), 1.75- 1.64 (m, 1H) |
| 851 | 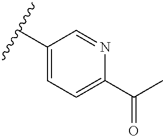 [SMILES: *c1ccc(C(C)=O)nc1] | (R)-N-(1-(7-(6- acetylpyridin-3-yl)-4- aminopyrrolo[2,1- f][1,2,4]triazin-5- yl)piperidin-3-yl)-2-(4- methylpiperazin-1- yl)pyrimidine-4- carboxamide | 556 | B: 1.13 | (500 MHz, DMSO-d₆) δ 9.40 (s, 1H), 8.72 (dd, J = 8.5, 1.8 Hz, 1H), 8.56 (d, J = 4.6 Hz, IH), 8.47 (br d, J = 8.5 Hz, 1H), 8.03 (d, J = 8.5 Hz, IH), 7.94 (s, 1H), 7.33 (s, 1H), 7.11 (d, J = 4.9 Hz, 1H), 4.27-4.18 (m, 1H), 3.81 (br s, 4H), 3.20 (br s, 1H), 3.10-3.02 (m, 1H), 2.96-2.87 (m, 1H), 2.84-2.75 (m, 1H), 2.66 (s, 3H), 2.39 (br d, J = 4.0 Hz, 4H), 2.23 (s, 3H), 1.92-1.83 (m, 3H), 1.76- 1.65 (m, 1H) |
| 852 | 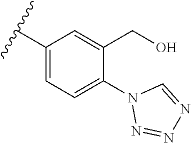 [SMILES: *c1ccc(-n2cnnn2)c(CO)c1] | (R)-N-(1-(4-amino-7-(3- (hydroxymethyl)-4-(1H- tetrazol-1- yl)phenyl)pyrrolo[2,1- f][1,2,4]triazin-5- yl)piperidin-3-yl)-2-(4- methylpiperazin-1- yl)pyrimidine-4- carboxamide, TFA salt | 611 | B: 1.01 | (500 MHz, DMSO-d₆) δ 9.84 (s, 1H), 8.67 (d, J = 4.9 Hz, 1H), 8.56 (br d, J = 8.5 Hz, 1H), 8.38 (s, 1H), 8.30-8.24 (m, 1H), 7.99 (s, 1H), 7.67 (d, J = 8.5 Hz, 1H), 7.26 (d, J = 4.6 Hz, 1H), 7.20 (s, 1H), 4.45 (s, 2H), 4.34-4.23 (m, 1H), 3.55 (br s, 4H), 3.34- 3.29 (m, 1H), 3.23 (br d, J = 7.9 Hz, 1H), 3.15-3.08 (m, 2H), 2.86 (s, 3H), 2.51 (br s, 4H), 1.96-1.85 (m, 3H), 1.75-1.64 (m, 1H) |
| 853 | 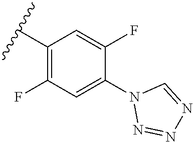 [SMILES: *c1cc(F)c(-n2cnnn2)cc1F] | (R)-N-(1-(4-amino-7-(2,5- difluoro-4-(1H-tetrazol-1- yl)phenyl)pyrrolo[2,1- f][1,2,4]triazin-5- yl)piperidin-3-y1)-2-(4- methylpiperazin-1- yl)pyrimidine-4- carboxamide | 617 | B: 1.21 | (500 MHz, DMSO-d₆) δ 9.99 (s, 1H), 8.56 (d, J = 4.9 Hz, 1H), 8.49-8.41 (m, 2H), 8.05 (dd, J = 10.5, 6.3 Hz, 1H), 7.91 (s, 1H), 7.11 (d, J = 4.9 Hz, 1H), 7.05 (d. J = 2.7 Hz, 1H), 4.27-4.17 (m, 1H), 3.81 (br s, 4H), 3.22-3.15 (m, 1H), 3.10-3.02 (m, 1H), 2.96-2.86 (m, 1H), 2.84-2.70 (m, 1H), 2.38 (br t, J = 4.7 Hz, 4H), 2.22 (s, 3H), 1.95-1.79 (m, 3H), 1.75-1.62 (m, 1H) |
| 854 | 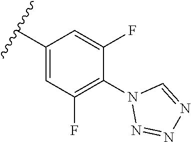 [SMILES: *c1cc(F)c(-n2cnnn2)c(F)c1] | (R)-N-(1-(4-amino-7-(3,5- difluoro-4-(1H-tetrazol-1- yl)phenyl)pyrrolo[2,1- f\|[1,2,4]triazin-5- yl)piperidin-3-yl)-2-(4- methylpiperazin-1- yl)pyrimidine-4- carboxamide | 617 | B: 1.22 | (500 MHz, DMSO-d₆) δ 10.02 (s, 1H), 8.57 (d, J = 4.9 Hz, 1H), 8.48 (br d, J = 8.2 Hz, 1H), 8.38 (br d, J = 10.7 Hz, 2H), 8.01 (s, 1H), 7.47 (s, 1H), 7.12 (d, J = 4.9 Hz, IH), 4.27-4.20 (m, 1H), 3.82 (br s, 4H), 3.22-3.16 (m, 1H), 3.11-3.04 (m, 1H), 2.93- 2.84 (m, 1H), 2.80-2.73 (m, 1H), 2.40 (br s, 4H), 2.24 (s, 3H), 1.93-1.84 (m, 3H), 1.76-1.69 (m, 1H) |
| 855 | 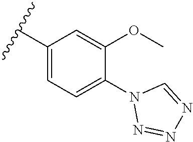 [SMILES: *c1ccc(-n2cnnn2)c(OC)c1] | (R)-N-(1-(4-amino-7-(3- methoxy-4-(1H-tetrazol-1- yl)phenyl)pyrrolo[2,1- f][1,2,4]triazin-5- yl)piperidin-3-yl)-2-(4- methylpiperazin-1- yl)pyrimidine-4- carboxamide | 611 | B: 1.16 | (500 MHz, DMSO-d₆) δ 9.83 (s, 1H), 8.57 (d, J = 4.6 Hz, 1H), 8.47 (br d, J = 8.2 Hz, 1H), 8.03-7.98 (m, 2H), 7.92 (s, 1H), 7.76 (d, J = 8.9 Hz, 1H), 7.27 (s, 1H), 7.12 (d, J = 4.9 Hz, 1H), 4.27- 4.20 (m, 1H), 3.96 (s, 3H), 3.82 (br s, 4H), 3.25-3.19 (m, 1H), 3.13-3.03 (m, 1H), 2.97-2.87 (m, 1H), 2.85-2.74 (m, 1H), 2.39 (br s, 4H), 2.23 (s, 3H), 1.94-1.83 (m, 3H), 1.76-1.67 (m, 1H) |
| 856 | 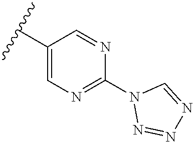 [SMILES: *c1cnc(-n2cnnn2)nc1] | (R)-N-(1-(7-(2-(1H- tetrazol-1-yl)pyrimidin-5- yl)-4-aminopyrrolo[2,1- f][1,2,4]triazin-5- yl)piperidin-3-yl)-2-(4- methylpiperazin-1- yl)pyrimidine-4- carboxamide | 583 | B: 1.03 | (500 MHz, DMSO-d₆) δ 10.26 (s, 1H), 9.71 (s, 2H), 8.57 (d, J = 4.9 Hz, 1H), 8.48 (br d, J = 8.2 Hz, 1H), 7.98 (s, IH), 7.44 (s, 1H), 7.11 (d, J = 4.6 Hz, 1H), 4.28-4.20 (m, 1H), 3.82 (br s, 4H), 3.22 (br d, J = 7.9 Hz, 1H), 3.08 (br d, J = 8.9 Hz, 1H), 2.91 (br dd, J = 10.7, 8.2 Hz, 1H), 2.82-2.73 (m, 1H), 2.39 (br d, J = 4.6 Hz, 4H), 2.24 (s, 3H), 1.95-1.83 (m, 3H), 1.71 (br d, J = 8.9 Hz, 1H) |
| 857 | 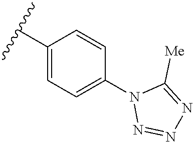 [SMILES: *c1ccc(-n2nnnc2C)cc1] | (R)-N-(1-(4-amino-7-(4-(5- methyl-1H-tetrazol-1- yl)phenyl)pyrrolo[2,1- f][1,2,4]triazin-5- yl)piperidin-3-yl)-2-(4- methylpiperazin-1- yl)pyrimidine-4- carboxamide | 595 | B: 1.14 | (500 MHz, DMSO-d₆) δ 8.57 (d, J = 4.6 Hz, 1H), 8.47 (br d, J = 8.2 Hz, 1H), 8.37 (d, J = 8.5 Hz, 2H), 7.91 (s, 1H), 7.76 (d, J = 8.5 Hz, 2H), 7.20 (s, 1H), 7.11 (d, J = 4.6 Hz, 1H), 4.23 (br dd, J = 8.5, 4.6 Hz, 1H), 3.81 (br s, 4H), 3.25-3.16 (m, 1H), 3.12- 3.03 (m, 1H), 2.95-2.86 (m, 1H), 2.84-2.75 (m, 1H), 2.61 (s, 3H), 2.39 (br t, J = 4.7 Hz, 4H), 2.23 (s, 3H), 1.94-1.82 (m, 3H), 1.76-1.64 (m, 1H) |
| 858 | 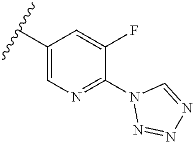 [SMILES: *c1cnc(-n2cnnn2)c(F)c1] | (R)-N-(1-(4-amino-7-(5- fluoro-6-(1H-tetrazol-]- yl)pyridin-3-yl)pyrrolo[2,1- f][1,2,4]triazin-5- yl)piperidin-3-yl)-2-(4- methylpiperazin-1- yl)pyrimidine-4- carboxamide | 600 | B: 1.12 | (500 MHz, DMSO-d₆) δ 10.11 (s, 1H), 9.26 (s, IH), 8.94 (br d, J = 12.2 Hz, 1H), 8.57 (d, J = 4.9 Hz, 1H), 8.48 (br d, J = 8.5 Hz, 1H), 8.00 (s, 1H), 7.47 (s, IH), 7.12 (d, J = 4.6 Hz, 1H), 4.29- 4.19 (m, 1H), 3.83 (br s, 4H), 3.22 (br d, J = 8.2 Hz, 1H), 3.13- 3.04 (m, 1H), 2.94-2.73 (m, 2H), 2.42 (br s, 4H), 2.26 (s, 3H), 1.96-1.83 (m, 3H), 1.78-1.66 (m, 1H) |
| 859 | 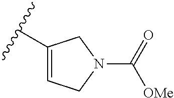 [SMILES: *C1=CCN(C(=O)OC)C1] | methyl (R)-3-(4-amino-5- (3-(2-(4-methylpiperazin-1- yl)pyrimidine-4- carboxamido)piperidin-1- yl)pyrrolo[2,1- f][1,2,4]triazin-7-yl)-2,5- dihydro-1H-pyrrole-1- | 562 | B: 1.15 | (500 MHz, DMSO-d₆) δ 8.55 (d, J = 4.9 Hz, 1H), 8.44 (br d, J = 8.5 Hz, 1H), 7.85 (s, 1H), 7.10 (d, J = 4.9 Hz, 1H), 6.74-6.69 (m, 2H), 4.54 (br s, 1H), 4.51 (br s, 1H), 4.33-4.27 (m, 2H), 4.17 (br d, J = 5.2 Hz, 1H), 3.80 (br s, 4H), 3.65 (br d, J = 5.2 Hz, 3H), 3.14-3.08 (m, 1H), 3.01-2.96 (m, 1H), 2.87-2.64 (m, 2H), 2.38 (br t, J = 4.6 Hz, 4H), 2.22 (s, 3H), 1.90-1.75 (m, 3H), 1.70-1.60 (m, 1H) |
| | | carboxylate | | | |

The following Examples in Table 61 were prepared using a similar procedure as described in Example 785 by using Intermediate 61 and 5-cyanothiophene-2-carboxylic acid, and then reacted with the appropriate boronic acids or boronates, prepared as intermediates described above, or obtained from commercial sources. Palladium catalysts other than PdCl₂(dppf) and solvents, such as dioxane/H₂O, THF/H₂O, or DMF/H₂O can alternatively be used.

TABLE 61

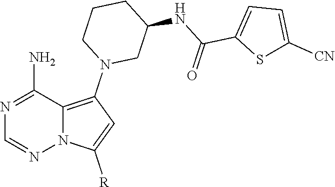

| | | | | HPLC | |
|---|---|---|---|---|---|
| | | | LCMS | Method, | |
| Ex- | | | (M + | RT | |
| ample | R | Name | H)⁺ | (min.) | ¹H NMR |
| 860 | 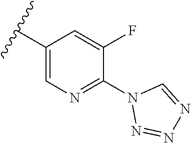 [SMILES: *c1cnc(-n2cnnn2)c(F)c1] | (R)-N-(1-(4-amino-7-(5- fluoro-6-(1H-tetrazol-1- yl)pyridin-3-yl)pyrrolo [2,1-f][1,2,4]triazin-5- yl)piperidin-3-yl)-5- cyanothiophene-2- carboxamide | 531 | B: 1.42 | (500 MHz, DMSO-d₆) δ 10.10 (br s, 1H), 9.25 (br s, 1H), 8.98-8.88 (m, 1H), 8.81 (br d, J = 7.2 Hz, 1H), 7.99 (br d, J = 1.8 Hz, 2H), 7.93 (br d, J = 2.9 Hz, 1H), 7.50-7.43 (m, 1H), 4.25-4.16 (m, 1H), 3.29-3.22 (m, 3.12-3.05 (m, 1H), 2.82-2.67 (m, 2H), 1H), 2.02-1.80 (m, 3H), 1.63-1.52 (m, 1H) |
| 861 | 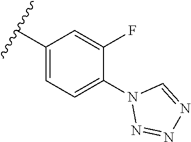 [SMILES: *c1ccc(-n2cnnn2)c(F)c1] | (R)-N-(1-(4-amino-7-(3- fluoro-4-(1H-tetrazol-1- yl)phenyl)pyrrolo[2,1- f][1,2,4]triazin-5- yl)piperidin-3-yl)-5- cyanothiophene-2- carboxamide | 530 | B: 1.39 | (500 MHz, DMSO-d₆) δ 9.98 (br d, J = 0.6 Hz, 1H), 8.87-8.76( m, 1H), 8.43 (br d, J = 12.2 Hz, 1H), 8.27-8.22 (m, 1H), 8.09- 7.91 (m, 4H), 7.36 (br s, 1H), 4.27-4.16 (m, 1H), 3.33-3.21 (m, 1H), 3.16-3.05 (m, 1H), 2.98-2.89 (m, 1H), 2.81-2.71 (m, 1H), 2.03-1.81 (m, 3H), 1.65-1.51 (m, 1H) |

### Example 862

### Preparation of (R)—N-(1-(4-amino-7-ethynylpyrrolo[2,1-f][1,2,4]triazin-5-yl)piperidin-3-yl)-5-chlorothiophene-2-carboxamide

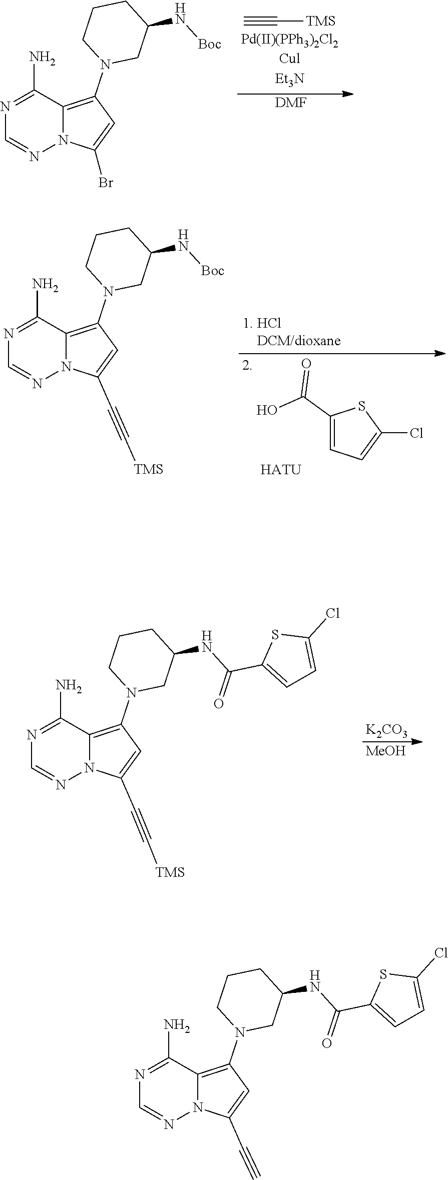

### Example 862. tert-butyl (R)-(1-(4-amino-7-((trimethylsilyl)ethynyl)pyrrolo[2,1-f][1,2,4]triazin-5-yl)piperidin-3-yl)carbamate

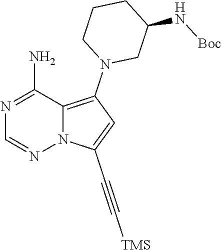 [SMILES: CC(C)(C)OC(=O)N[C@@H]1CCCN(c2cc(C#C[Si](C)(C)C)n3ncnc(N)c23)C1]

A mixture of tert-butyl (R)-(1-(4-amino-7-bromopyrrolo[2,1-f][1,2,4]triazin-5-yl)piperidin-3-yl)carbamate (530 mg, 1.29 mmol), ethynyltrimethylsilane (0.71 mL, 5.15 mmol), bis(triphenylphosphine)palladium(II) chloride (145 mg, 0.206 mmol), and copper(I) iodide (82 mg, 0.431 mmol), in 2:1 triethylamine/DMF (6 mL) was degassed by sparging with nitrogen for 15 minutes. The vial was capped, and the reaction was stirred at 80° C., for 18 hours. The mixture was diluted with ethyl acetate (50 mL) and filtered, and the filtrate was washed with water, 10% aq. LiCl (2×), and brine, then dried (sodium sulfate) and concentrated. The residue was purified by flash chromatography (5% to 100% ethyl acetate/hexanes gradient) to afford the title compound (208 mg, 38% yield). LC-MS [M+H]⁺=429.6; ¹H NMR (500 MHz, CDCl₃) δ 8.02-7.82 (m, 1H), 6.70 (s, 1H), 1.46 (s, 9H), 0.33-0.29 (m, 9H).

### Example 862. (R)—N-(1-(4-amino-7-((trimethylsilyl)ethynyl)pyrrolo[2,1-f][1,2,4]triazin-5-yl)piperidin-3-yl)-5-chlorothiophene-2-carboxamide

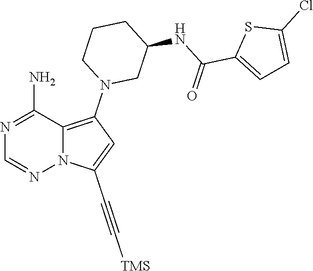 [SMILES: C[Si](C)(C)C#Cc1cc(N2CCC[C@@H](NC(=O)c3ccc(Cl)s3)C2)c2c(N)ncnn12]

A solution of tert-butyl (R)-(1-(4-amino-7-((trimethylsilyl)ethynyl)pyrrolo[2,1-f][1,2,4]triazin-5-yl)piperidin-3-yl)carbamate (758 mg, 1.77 mmol) in dichloromethane (5 mL) was treated with 4M HCl in dioxane (3 mL), and the reaction was stirred at rt for 1 h. The mixture was concentrated, and the residue was concentrated twice from dichloromethane to remove residual HCl. The residue was taken up in dichloromethane, (5 mL), and the solution was treated with 5-chlorothiophene-2-carboxylic acid (288 mg, 1.77 mmol). The mixture was cooled to 0° C., and treated with triethylamine (1.48 mL, 10.6 mmol) and HATU (672 mg, 1.77 mmol). The reaction was stirred at rt for 18 hours. The mixture was diluted with ethyl acetate, then washed with water and brine, dried (sodium sulfate) and concentrated in vacuo. The residue was purified by flash chromatography (5% to 100% EtOAc/hexanes gradient) to yield the title compound (695 mg, 83% yield). MS: [M+H]⁺=473.0; ¹H NMR (500 MHz, CDCl₃) δ 7.95 (s, 1H), 7.28 (d, J=4.1 Hz, 1H), 6.92 (d, J=3.9 Hz, 1H), 6.71 (s, 1H), 6.14-5.65 (m, 2H), 4.29 (br d, J=1.2 Hz, 1H), 3.54-3.28 (m, 1H), 3.17-2.98 (m, 1H), 2.92-2.60 (m, 2H), 2.13-2.00 (m, 1H), 1.92 (br dd, J=9.7, 4.7 Hz, 1H), 1.87-1.78 (m, 1H), 0.32-0.30 (m, 9H).

### Example 862

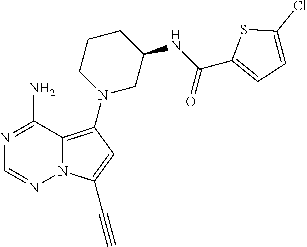 [SMILES: C#Cc1cc(N2CCC[C@@H](NC(=O)c3ccc(Cl)s3)C2)c2c(N)ncnn12]

A solution of (R)—N-(1-(4-amino-7-((trimethylsilyl)ethynyl)pyrrolo[2,1-f][1,2,4]triazin-5-yl)piperidin-3-yl)-5-chlorothiophene-2-carboxamide (695 mg, 1.47 mmol) in methanol (5 mL) at 0° C. was treated with potassium carbonate (102 mg, 0.735 mmol). The reaction was stirred at 0° C. for 20 minutes. The mixture was diluted with ethyl acetate (30 mL) and filtered, and the filtrate was concentrated in vacuo. The residue was purified by flash chromatography (0% to 10% methanol/dichloromethane gradient) to yield the title compound (470 mg, 80% yield). A portion of the material was further purified via preparative LC/MS. LC-MS Method A: RT=1.75 min, [M+H]⁺=400.9; LC-MS Method B: RT=1.52 min, [M+H]⁺=401.0; ¹H NMR (500 MHz, DMSO-d₆) δ 8.47 (br d, J=7.6 Hz, 1H), 8.15-7.93 (m, 1H), 7.89-7.77 (m, 1H), 7.68 (br d, J=2.6 Hz, 1H), 7.16 (dt, J=4.1, 2.2 Hz, 1H), 7.00-6.86 (m, 1H), 6.84 (s, 1H), 4.72-4.53 (m, 1H), 4.09 (br dd, J=6.3, 2.4 Hz, 1H), 3.82-3.59 (m, 1H), 3.22-3.04 (m, 1H), 3.00-2.86 (m, 1H), 2.73-2.57 (m, 1H), 1.95-1.70 (m, 3H), 1.56-1.40 (m, 1H).

### Example 863 and Example 864

### Preparation of tert-butyl (R)-3-(4-(4-amino-5-(3-(5-chlorothiophene-2-carboxamido)piperidin-1-yl)pyrrolo[2,1-f][1,2,4]triazin-7-yl)-1H-1,2,3-triazol-1-yl)propanoate and (R)-3-(4-(4-amino-5-(3-(5-chlorothiophene-2-carboxamido)piperidin-1-yl)pyrrolo[2,1-f][1,2,4]triazin-7-yl)-1H-1,2,3-triazol-1-yl)propanoic acid

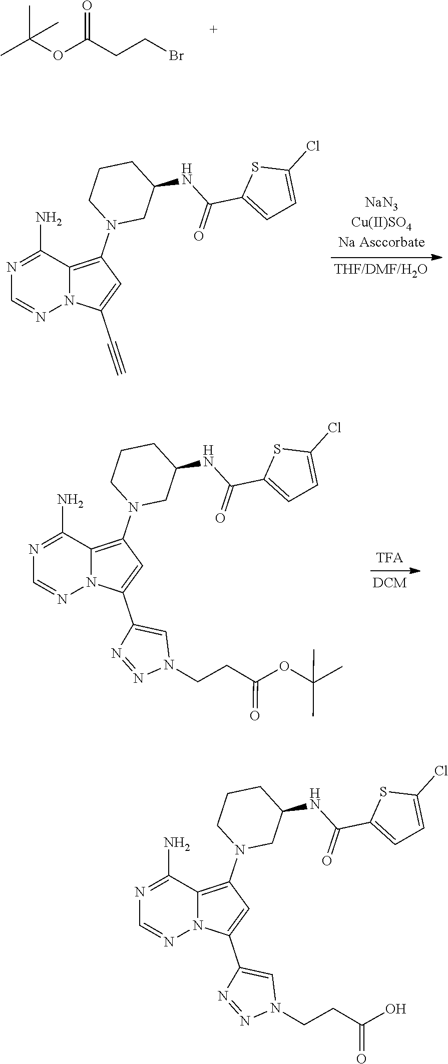

A mixture of tert-butyl 3-bromopropanoate (56.3 mg, 0.269 mmol) and sodium azide (17.0 mg, 0.262 mmol) in DMF (0.1 mL) was stirred at 85° C. for 18 h. The mixture was transferred to a vial containing a solution of (R)—N-(1-(4-amino-7-ethynylpyrrolo[2,1-f][1,2,4]triazin-5-yl)piperidin-3-yl)-5-chlorothiophene-2-carboxamide (30 mg, 0.075 mmol) in 2:1 THF/water (0.3 mL). The solution was treated with copper(II) sulfate (0.51M aq.) (0.085 mL, 0.045 mmol), and sodium ascorbate (0.62M aq.) (0.024 mL, 0.015 mmol), the vial was sealed, and the reaction was stirred at 50° C. for 3 h. The mixture was diluted with THF (1 mL), then treated with 0.5M EDTA (pH 8) solution. The heterogeneous mixture was stirred vigorously for 30 minutes, then poured into ethyl acetate (10 mL). The layers were separated, and the organic phase was washed sequentially with EDTA solution and brine, then dried (sodium sulfate) and concentrated. Half of the residue was purified via preparative LC/MS to yield Example 863. Method A: RT=2.02 min, [M+H]⁺=572.2; LC-MS Method B: RT=1.56 min, [M+H]⁺=572.3; ¹H NMR (500 MHz, DMSO-d₆) δ 8.64 (s, 1H), 8.49 (br d, J=7.9 Hz, 1H), 7.99 (s, 1H), 7.72 (d, J=4.0 Hz, 1H), 7.19 (d, J=4.0 Hz, 1H), 7.14-7.09 (m, 1H), 4.67 (br t, J=6.4 Hz, 2H), 4.18 (br d, J=3.7 Hz, 1H), 3.29-3.14 (m, 1H), 3.13-3.03 (m, 1H), 2.92 (br t, J=6.4 Hz, 2H), 2.85-2.65 (m, 2H), 2.01-1.81 (m, 3H), 1.62-1.45 (m, 1H), 1.41-1.29 (m, 9H).

### Example 864

The remainder of the crude material from Example 863 was taken up in dichloromethane (2 mL). The stirring solution was treated with TFA (1 mL), and the reaction was stirred at rt for 2 h. The mixture was concentrated, and the residue was purified via preparative LC/MS to yield Example 864. LC-MS Method A: RT=1.27 min, [M+H]⁺=516.2; LC-MS Method B: RT=1.33 min, [M+H]⁺=516.4; ¹H NMR (500 MHz, DMSO-d₆) δ 8.63 (s, 1H), 8.49 (br d, J=7.3 Hz, 1H), 7.98 (s, 1H), 7.72 (d, J=4.0 Hz, 1H), 7.19 (d, J=4.3 Hz, 1H), 7.10 (s, 1H), 4.67 (br t, J=6.4 Hz, 2H), 4.27-4.08 (m, 1H), 3.30-3.20 (m, 1H), 3.15-3.05 (m, 1H), 2.95 (br t, J=6.6 Hz, 2H), 2.85-2.69 (m, 2H), 2.05-1.79 (m, 4H), 1.60-1.42 (m, 1H).

### Example 865

### Preparation of (R)—N-(1-(4-amino-7-(1-(2-amino-2-oxoethyl)-1H-1,2,3-triazol-4-yl)pyrrolo[2,1-f][1,2,4]triazin-5-yl)piperidin-3-yl)-5-chlorothiophene-2-carboxamide

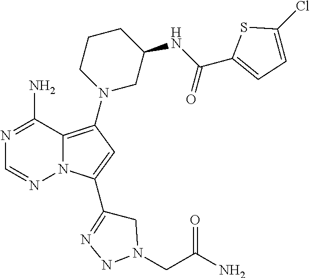 [SMILES: NC(=O)CN1CC(c2cc(N3CCC[C@@H](NC(=O)c4ccc(Cl)s4)C3)c3c(N)ncnn23)=NN1]

According to the procedure for the preparation of Example 863, reaction of Example 862 and 2-bromoacetamide afforded Example 865. LC-MS Method A: RT=1.47 min, [M+H]⁺=501.1; LC-MS Method B: RT=1.25 min, [M+H]⁺=501.1; ¹H NMR (500 MHz, DMSO-d₆) δ 8.60 (s, 1H), 8.48 (br d, J=7.9 Hz, 1H), 7.91 (s, 1H), 7.76 (br s, 1H), 7.72 (br d, J=4.0 Hz, 1H), 7.40 (br s, 1H), 7.18 (d, J=4.0 Hz, 1H), 7.08 (s, 1H), 5.22-5.14 (m, 2H), 4.23-4.09 (m, 1H), 3.42-3.29 (m, 1H), 3.26-3.16 (m, 1H), 3.13-3.02 (m, 1H), 2.84-2.67 (m, 2H), 1.98-1.73 (m, 3H), 1.63-1.43 (m, 1H).

### Example 866

### Preparation of N-((3R)-1-(4-amino-7-(1-(pyrrolidin-3-yl)-1H-1,2,3-triazol-4-yl)pyrrolo[2,1-f][1,2,4]triazin-5-yl)piperidin-3-yl)-5-chlorothiophene-2-carboxamide 7%

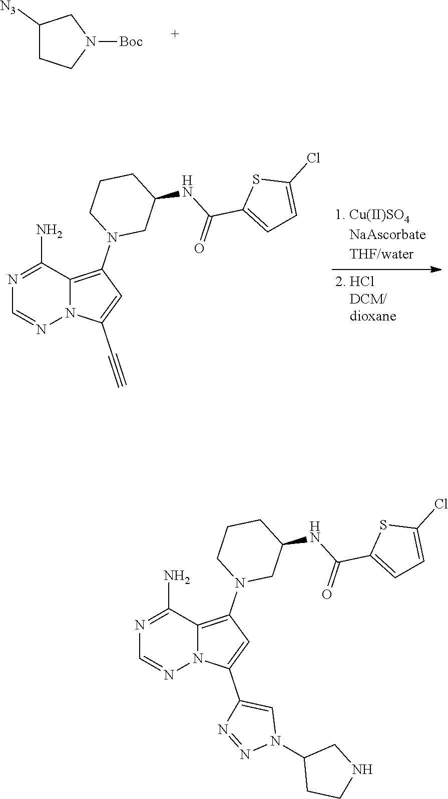 [SMILES: CC(C)(C)OC(=O)N1CCC(N=[N+]=[N-])C1 ; C#Cc1cc(N2CCC[C@@H](NC(=O)c3ccc(Cl)s3)C2)c2c(N)ncnn12 ; Nc1ncnn2c(-c3cn(C4CCNC4)nn3)cc(N3CCC[C@@H](NC(=O)c4ccc(Cl)s4)C3)c12]

In a 2-dram vial, a solution of (R)—N-(1-(4-amino-7-ethynylpyrrolo[2,1-f][1,2,4]triazin-5-yl)piperidin-3-yl)-5-chlorothiophene-2-carboxamide (30 mg, 0.075 mmol) in 9:1 THF/water (0.3 mL) was treated with tert-butyl 3-azidopyrrolidine-1-carboxylate (17.5 mg, 0.082 mmol), copper(II) sulfate (0.51 M aq.) (0.042 mL, 0.022 mmol), and aq. sodium ascorbate (0.62 M) (0.012 mL, 7.5 μmol). The vial was sealed, and the reaction was stirred at 50° C. The mixture was diluted with THF (1 mL), then treated with 0.5M EDTA (pH 8) solution. The heterogeneous mixture was stirred vigorously for 30 minutes, then poured into ethyl acetate (10 mL). The layers were separated, and the organic phase was washed sequentially with EDTA solution and brine, then dried (sodium sulfate) and concentrated. The residue was taken up in dichloromethane (2 mL), and the mixture was treated with TFA (1 mL). The reaction was stirred at rt for 1 h, then concentrated. The crude material was purified via preparative HPLC to yield the title compound. LC-MS Method A: RT=1.34 min, [M+H]⁺=513.3; LC-MS Method B: RT=1.17 min, [M+H]⁺=512.9; ¹H NMR (500 MHz, DMSO-d₆) δ 8.62 (s, 1H), 8.48 (br d, J=7.9 Hz, 1H), 8.07-7.94 (m, 1H), 7.90 (s, 1H), 7.72 (d, J=4.0 Hz, 1H), 7.18 (d, J=4.0 Hz, 1H), 7.06 (s, 1H), 6.97-6.71 (m, 1H), 5.34-5.18 (m, 1H), 4.25-4.06 (m, 1H), 3.49-3.26 (m, 1H), 3.25-3.18 (m, 1H), 3.15-3.08 (m, 1H), 3.08-3.02 (m, 1H), 2.99-2.91 (m, 1H), 2.83-2.65 (m, 2H), 2.40-2.30 (m, 1H), 2.20-2.07 (m, 1H), 1.99-1.75 (m, 3H), 1.61-1.43 (m, 1H).

### Example 867

### Preparation of (R)-2-(4-(4-amino-5-(3-(5-chlorothiophene-2-carboxamido)piperidin-1-yl)pyrrolo[2,1-f][1,2,4]triazin-7-yl)-1H-1,2,3-triazol-1-yl)acetic acid

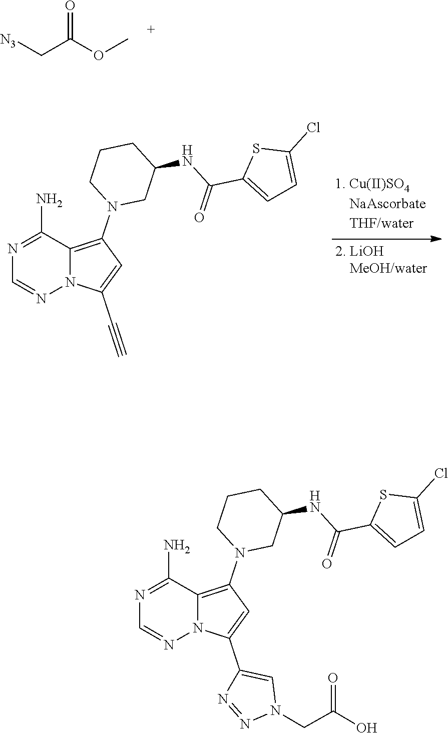

A solution of (R)—N-(1-(4-amino-7-ethynylpyrrolo[2,1-f][1,2,4]triazin-5-yl)piperidin-3-yl)-5-chlorothiophene-2-carboxamide (30 mg, 0.075 mmol) in 9:1 THF/water (0.3 mL) was treated with methyl 2-azidoacetate (8.8 μl, 0.090 mmol), copper(II) sulfate (0.51M aq.) (0.042 mL, 0.022 mmol), and aq. sodium ascorbate (0.62M) (0.012 mL, 7.5 μmol). The vial was scaled, and the reaction was stirred at 50° C. The mixture was diluted with THF (1 mL), then treated with 0.5M EDTA (pH 8) solution. The heterogeneous mixture was stirred vigorously for 30 minutes, then poured into ethyl acetate (10 mL). The layers were separated, and the organic phase was washed sequentially with EDTA solution and brine, then dried (sodium sulfate) and concentrated. In a 2-dram vial, half of the residue was taken up in methanol (1 mL). The mixture was treated with 0.5 M lithium hydroxide (0.228 mL, 0.114 mmol). The vial was sealed, and the reaction was stirred at 50° C. for 2 h. The mixture was adjusted to pH 3 with 1M HCl, then extracted 3× with ethyl acetate. The organic phases were combined, dried over sodium sulfate, and concentrated. The crude material was purified via preparative HPLC to yield the title compound. LC-MS Method A: RT=1.31 min, [M+H]⁺=502.0; LC-MS Method B: RT=1.38 min, [M+H]⁺=502.2; ¹H NMR (500 MHz, DMSO-d₆) δ 8.65 (s, 1H), 8.49 (br d, J=7.6 Hz, 1H), 7.89 (s, 1H), 7.71 (br d, J=3.9 Hz, 1H), 7.17 (d, J=4.0 Hz, 1H), 7.09 (s, 1H), 5.36 (s, 2H), 4.22-4.07 (m, 1H), 3.57-3.16 (m, 1H), 3.10-3.00 (m, 1H), 2.86-2.67 (m, 2H), 2.00-1.76 (m, 3H), 1.61-1.46 (m, 1H).

### Example 868

### Preparation of (R)—N-(1-(4-amino-7-(1-methyl-1H-1,2,3-triazol-4-yl)pyrrolo[2,1-f][1,2,4]triazin-5-yl)piperidin-3-yl)-5-chlorothiophene-2-carboxamide

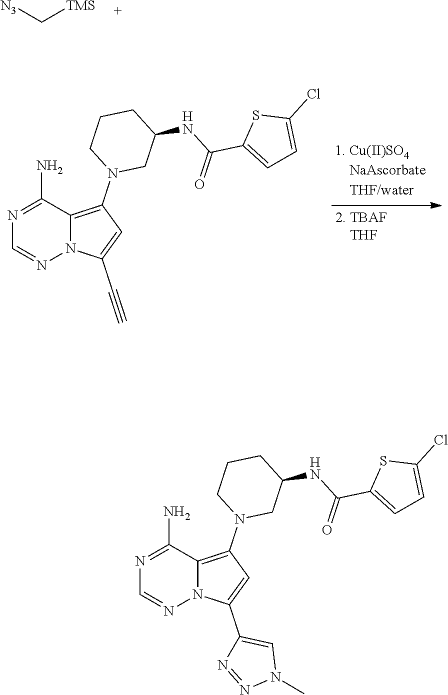 [SMILES: C[Si](C)(C)CN=[N+]=[N-] ; C#Cc1cc(N2CCC[C@@H](NC(=O)c3ccc(Cl)s3)C2)c2c(N)ncnn12 ; Cn1cc(-c2cc(N3CCC[C@@H](NC(=O)c4ccc(Cl)s4)C3)c3c(N)ncnn23)nn1]

In a 2-dram vial, a solution of (R)—N-(1-(4-amino-7-ethynylpyrrolo[2,1-f][1,2,4]triazin-5-yl)piperidin-3-yl)-5-chlorothiophene-2-carboxamide (20 mg, 0.050 mmol) in 9:1 THF/water (0.2 mL) was treated with (azidomethyl)trimethylsilane (8.89 μl, 0.060 mmol), copper(II) sulfate (0.51M aq.) (0.028 mL, 0.015 mmol), and sodium ascorbate (0.62M aq.) (8.1 μl, 5.0 μmol). The vial was scaled, and the reaction was stirred at 50° C. The mixture was diluted with THF (0.5 mL), then treated with 0.5M EDTA (pH 8) solution. The heterogeneous mixture was stirred vigorously for 30 minutes, then poured into ethyl acetate (5 mL). The layers were separated, and the organic phase was washed sequentially with EDTA solution and brine, then dried (sodium sulfate) and concentrated. The residue was taken up in THF (0.2 mL), and treated with TBAF (0.100 mL, 0.100 mmol). The reaction was stirred at rt for 2 h. The mixture was diluted with ethyl acetate (5 mL), and the solution was washed with water (3×) and brine, dried (sodium sulfate) and concentrated. The crude material was purified via preparative HPLC to yield the title compound (12 mg, 52% yield). LC-MS Method A: RT=1.54 min, [M+H]⁺=458.1; LC-MS Method B: RT=1.41 min, [M+H]⁺=458.2; ¹H NMR (500 MHz, DMSO-d) δ 8.60 (s, 1H), 8.51 (br d, J=7.9 Hz, 1H), 7.98 (s, 1H), 7.70 (d, J=4.3 Hz, 1H), 7.17 (d, J=4.0 Hz, 1H), 7.12 (s, 1H), 4.22-4.08 (m, 4H), 3.55-3.44 (m, 1H), 3.27-3.20 (m, 1H), 3.12-3.04 (m, 1H), 2.84-2.69 (m, 2H), 1.99-1.78 (m, 3H), 1.52 (dt, J=6.3, 3.0 Hz, 1H).

### Example 869

### Preparation of (R)-3-((1-(7-(1-acetyl-2,5-dihydro-1H-pyrrol-3-yl)-4-aminopyrrolo[2,1-f][1,2,4]triazin-5-yl)piperidin-3-yl)carbamoyl)benzenesulfonyl fluoride

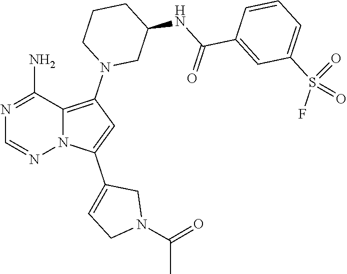 [SMILES: CC(=O)N1CC=C(c2cc(N3CCC[C@@H](NC(=O)c4cccc(S(=O)(=O)F)c4)C3)c3c(N)ncnn23)C1]

According to the procedure for the preparation of Example 255, reaction of Example 255C with 3-(fluorosulfonyl)benzoic acid afforded Example 869. LC-MS Method A: RT=1.62 min, [M+H]⁺=528.3; LC-MS Method B: RT=1.38 min, [M+H]⁺=528.1; ¹H NMR (500 MHz, DMSO-d₆) δ 8.81 (br d, J=8.0 Hz, 1H), 8.54 (br s, 1H), 8.39 (br d, J=7.4 Hz, 1H), 8.30 (br d, J=7.9 Hz, 1H), 7.95 (s, 1H), 7.91 (br t, J=7.8 Hz, 1H), 6.86-6.73 (m, 2H), 4.70 (br s, 1H), 4.56-4.44 (m, 21H), 4.32-4.17 (m, 2H), 3.30-3.20 (m, 1H), 3.11-3.00 (m, 1H), 2.81-2.66 (m, 2H), 2.10-1.79 (m, 6H), 1.65-1.48 (m, 1H).

### Example 870

### Preparation of (R)-4-((1-(7-(1-acetyl-2,5-dihydro-1H-pyrrol-3-yl)-4-aminopyrrolo[2,1-f][1,2,4]triazin-5-yl)piperidin-3-yl)carbamoyl)benzenesulfonyl fluoride

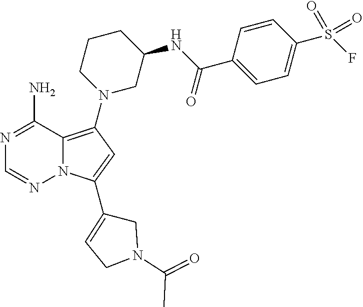 [SMILES: CC(=O)N1CC=C(c2cc(N3CCC[C@@H](NC(=O)c4ccc(S(=O)(=O)F)cc4)C3)c3c(N)ncnn23)C1]

According to the procedure for the preparation of Example 255, reaction of Example 255C with 4-(fluorosulfonyl)benzoic acid afforded Example 870. LC-MS Method A: RT=1.59 min, [M+H]⁺=528.2; LC-MS Method B: RT=1.36 min, [M+H]⁺=528.1; 1H NMR (500 MHz, DMSO-d6) δ 8.80 (br d, J=8.0 Hz, 1H), 8.23 (br d, J=7.4 Hz, 2H), 8.14 (br d, J=7.7 Hz, 2H), 7.96 (s, 1H), 6.88-6.73 (m, 2H), 4.69 (br s, 1H), 4.49 (br d, J=14.7 Hz, 2H), 4.34-4.17 (m, 2H), 3.79-3.64 (m, 1H), 3.29-3.19 (m, 1H), 3.09-2.97 (m, 1H), 2.82-2.67 (m, 2H), 2.08-1.98 (m, 3H), 1.97-1.78 (m, 3H), 1.63-1.49 (m, 1H).

### Example 871

### Preparation of (R)—N-(1-(4-amino-7-(benzo[d]isoxazol-6-yl)pyrrolo[2,1-f][1,2,4]triazin-5-yl)piperidin-3-yl)-5-chlorothiophene-2-carboxamide

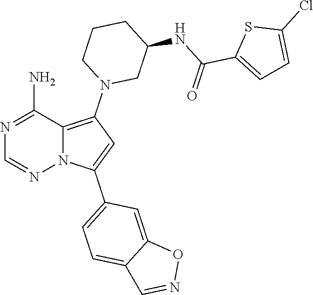 [SMILES: Nc1ncnn2c(-c3ccc4cnoc4c3)cc(N3CCC[C@@H](NC(=O)c4ccc(Cl)s4)C3)c12]

The title compound can be prepared using the procedures described in Example 255, substituting 6-(4,4,5,5-tetramethyl-1,3,2-dioxaborolan-2-yl)benzo[d]isoxazole (prepared from 6-bromobenzo[d]isoxazole via the procedure used for Intermediate 1) for Example 255A in step B, and 5-chlorothiophene-2-carboxylic acid for 5-methylthiophene-2-carboxylic acid in the final step. LC-MS Method A: RT=1.78 min, [M+H]⁺=494.2; LC-MS Method B: RT=1.58 min, [M+H]⁺=494.2; 1H NMR (500 MHz, DMSO-d₆) δ 8.49 (br d, J=7.9 Hz, 1H), 7.96 (d, J=9.2 Hz, 2H), 7.71 (d, J=4.0 Hz, 1H), 7.66 (d, J=8.2 Hz, 1H), 7.54 (br d, J=7.6 Hz, 1H), 7.18 (d, J=4.0 Hz, 1H), 7.15 (s, 1H), 4.23-4.07 (m, 1H), 3.30-3.04 (m, 1H), 2.82-2.62 (m, 2H), 2.02-1.77 (m, 4H), 1.62-1.44 (m, 1H).

### Example 872

### Preparation of (R)—N-(1-(4-amino-7-(benzo[d]isoxazol-6-yl)pyrrolo[2,1-f][1,2,4]triazin-5-yl)piperidin-3-yl)-5-chloro-3-(2-(dimethylamino)ethoxy)thiophene-2-carboxamide

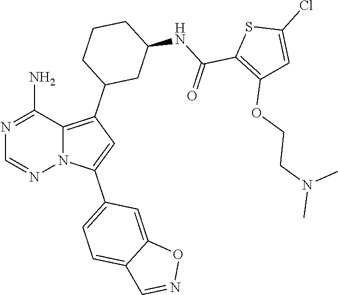 [SMILES: CN(C)CCOc1cc(Cl)sc1C(=O)N[C@@H]1CCCC(c2cc(-c3ccc4cnoc4c3)n3ncnc(N)c23)C1]

The title compound can be prepared using the procedures described in Example 871, substituting Example 141B for 5-chlorothiophene-2-carboxylic acid in the final step. LC-MS Method A: RT=1.67 min, [M+H]⁺=581.0; LC-MS Method B: RT=1.23 min, [M+H]⁺=581.0; ¹H NMR (500 MHz, DMSO-d₆) δ 8.00 (s, 1H), 7.90 (s, 1H), 7.70 (br d, J=7.3 Hz, 1H), 7.66-7.60 (m, 1H), 7.55 (d, J=8.5 Hz, 1H), 7.35 (s, 1H), 7.12 (s, 1H), 4.31 (br d, J=4.6 Hz, 2H), 4.26-4.10 (m, 1H), 3.37 (br d, J=5.8 Hz, 2H), 2.55 (s, 8H), 2.33-2.16 (m, 4H), 1.95-1.76 (m, 4H).

### Example 873

### Preparation of phenyl (R)-(1-(7-(1-acetyl-2,5-dihydro-1H-pyrrol-3-yl)-4-aminopyrrolo[2,1-f][1,2,4]triazin-5-yl)piperidin-3-yl)carbamate

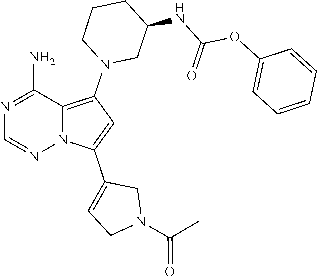 [SMILES: CC(=O)N1CC=C(c2cc(N3CCC[C@@H](NC(=O)Oc4ccccc4)C3)c3c(N)ncnn23)C1]

A solution of Example 255C (210 mg, 0.556 mmol) and DIEA (0.243 mL, 1.39 mmol) in dichloromethane (3 mL) was treated with phenyl carbonochloridate (0.077 mL, 0.61 mmol). The reaction was stirred at rt for 18 h. The mixture was treated with methanol and concentrated. The residue was purified by flash chromatography (0% to 15% methanol/dichloromethane gradient) to yield the title compound. LC-MS Method A: RT=1.57 min, [M+H]⁺=462.1; LC-MS Method B: RT=1.28 min, [M+H]⁺=462.2; ¹H NMR (500 MHz, DMSO-d₆) δ 8.12-7.99 (m, 1H), 7.94 (s, 1H), 7.42-7.34 (m, 2H), 7.27-7.17 (m, 2H), 7.15-7.02 (m, 3H), 6.84-6.73 (m, 2H), 4.71 (br s, 1H), 4.50 (br d, J=18.3 Hz, 2H), 4.28 (br s, 1H), 3.90-3.76 (m, 1H), 3.27-3.10 (m, 1H), 3.01-2.63 (m, 2H), 2.14-2.00 (m, 3H), 1.97-1.83 (m, 2H), 1.80-1.65 (m, 1H), 1.54-1.33 (m, 1H).

### Example 874

### Preparation of (R)—N—((R)-1-(7-(1-acetyl-2,5-dihydro-1H-pyrrol-3-yl)-4-aminopyrrolo[2,1-f][1,2,4]triazin-5-yl)piperidin-3-yl)-3-hydroxypyrrolidine-1-carboxamide

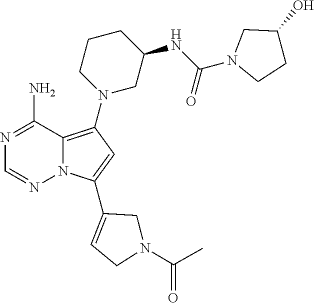 [SMILES: CC(=O)N1CC=C(c2cc(N3CCC[C@@H](NC(=O)N4CC[C@@H](O)C4)C3)c3c(N)ncnn23)C1]

A mixture of Example 873 and (R)-pyrrolidin-3-ol (11.3 mg, 0.130 mmol) in DMF (0.2 mL) was stirred at 150° C. for 1 h. The mixture was purified via preparative LC/MS to yield the title compound (6.1 mg, 52% yield). LC-MS Method A: RT=1.13 min, [M+H]⁺=455.2; LC-MS Method B: RT=0.99 min, [M+H]⁺=455.1 ¹H NMR (500 MHz, DMSO-d₆) δ 8.03-7.73 (m, 2H), 6.91-6.71 (m, 2H), 6.67 (br d, J=14.6 Hz, 1H), 5.83 (br d, J=7.0 Hz, 1H), 4.69 (br s, 1H), 4.58-4.40 (m, 2H), 4.25 (br d, J=16.5 Hz, 2H), 3.79 (br s, 1H), 3.46-3.06 (m, 2H), 3.00-2.92 (m, 1H), 2.77-2.58 (m, 1H), 2.51 (br d, J=1.5 Hz, 2H), 2.11-1.99 (m, 3H), 1.92-1.65 (m, 5H), 1.50-1.36 (m, 1H).

The following examples in Table 62 were prepared using the same procedure as shown in Example 874. Example 873 was reacted with the appropriate amin.

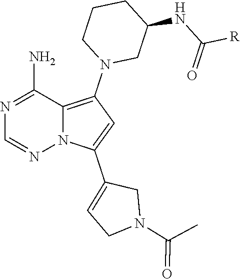

TABLE 62

| | | | | HPLC | |
|---|---|---|---|---|---|
| Ex- | | | LCMS | Method, | |
| am- | | | (M + | RT | |
| ple | R | Name | H)⁺ | (min.) | ¹H NMR |
| 875 | 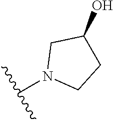 [SMILES: *N1CC[C@H](O)C1] | (S)-N-((R)-1-(7-(1-acetyl-2,5- dihydro-1H-pyrrol-3-yl)-4- aminopyrrolo[2,1- f][1,2,4]triazin-5-yl)piperidin- 3-yl)-3-hydroxypyrrolidine-1- carboxamide | 455.1 | A: 1.12 B: 0.99 | (500 MHz, DMSO-d₆) δ 7.95 (s, 1H), 6.88-6.66 (m, 2H), 5.86 (br d, J = 7.0 Hz, 1H), 4.71 (br s, 1H), 4.62-4.47 (m, 2H), 4.26 (br d, J = 18.6 Hz, 2H), 3.92-3.73 (m, 1H), 3.41-3.22 (m, 2H), 3.13 (br d, J = 10.7 Hz, 2H), 3.03-2.93 (m, 1H), 2.70-2.57 (m, 1H), 2.55 (s, 5H), 2.11-1.97 (m, 3H), 1.92-1.68 (m, 5H), 1.49-1.36 (m, 1H). |
| 876 | 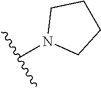 [SMILES: *N1CCCC1] | (R)-N-(1-(7-(1-acctyl-2,5- dihydro-1H-pyrrol-3-yl)-4- aminopyrrolo[2,1- f][1,2,4]triazin-5-yl)piperidin- 3-yl)pyrrolidine-1- carboxamide | | A: 1.30 B: 1.11 | (500 MHz, DMSO-d₆) δ 7.86 (s, IH), 6.77 (br d, J = 12.9 Hz, 2H), 6.66 (d. J = 12.1 Hz, 1H), 5.81 (br d, J = 7.0 Hz, 1H), 4.69 (br s, 1H), 4.48 (br d, J = 11.8 Hz, 2H), 4.26 (br s, 1H), 3.87-3.71 (m, 1H), 3.51- 3.39 (m, 1H), 3.20 (br s, 3H), 3.14-3.05 (m, 1H), 3.01-2.91 (m, 1H), 2.70-2.57 (m, 1H), 2.10-1.98 (m, |
| | | | | | 3H), 1.88-1.67 (m, 7H), 1.53-1.32 (m, 1H). |

### Example 879

### (R)—N-(1-(7-(1-acetyl-2,5-dihydro-1H-pyrrol-3-yl)-4-aminopyrrolo[2,1-f][1,2,4]triazin-5-yl)piperidin-3-yl)isoxazole-3-carboxamide

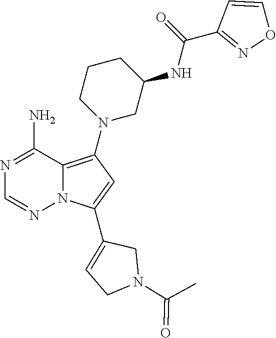 [SMILES: CC(=O)N1CC=C(c2cc(N3CCC[C@@H](NC(=O)c4ccon4)C3)c3c(N)ncnn23)C1]

According to the synthesis or Example 255, coupling with isoxazole-3-carboxylic acid afforded the title compound. LC-MS Method A: RT=1.35 min, [M+H]⁺=437.1; LC-MS Method B: RT=1.12 min, [M+H]⁺=437.1; ¹H NMR (500 MHz, DMSO-d₆) δ 9.04 (s, 1H), 8.78 (br dd, J=7.6, 4.6 Hz, 1H), 7.86 (d, J=2.4 Hz, 1H), 6.88 (s, 1H), 6.77 (br d, J=13.4 Hz, 1H), 6.70 (d, J=11.3 Hz, 1H), 4.69 (br s, 1H), 4.54-4.42 (m, 2H), 4.27 (br s, 1H), 4.18 (br d, J=6.1 Hz, 1H), 3.25-3.11 (m, 1H), 3.07-2.89 (m, 1H), 2.84-2.61 (m, 2H), 2.09-2.00 (m, 3H), 1.92-1.72 (m, 3H), 1.64-1.49 (m, 1H).

### Example 880

### Preparation of (R)—N-(1-(4-amino-7-(1-(thiazole-2-carbonyl)-2,5-dihydro-1H-pyrrol-3-yl)pyrrolo[2,1-f][1,2,4]triazin-5-yl)piperidin-3-yl)-5-chlorothiophene-2-carboxamide

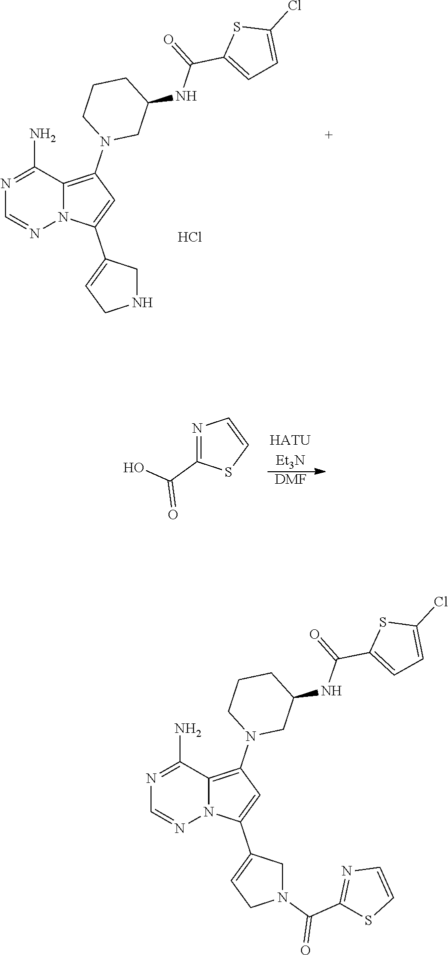

A solution of (R)—N-(1-(4-amino-7-(2,5-dihydro-1H-pyrrol-3-yl)pyrrolo[2,1-f][1,2,4]triazin-5-yl)piperidin-3-yl)-5-chlorothiophene-2-carboxamide hydrochloride (15 mg, 0.031 mmol) (prepared from Example 203C and tert-butyl 3-(4,4,5,5-tetramethyl-1,3,2-dioxaborolan-2-yl)-2,5-dihydro-1H-pyrrole-1-carboxylate using the conditions described for Example 203, followed by HCl-mediated removal of the Boc-group), thiazole-2-carboxylic acid (4.4 mg, 0.034 mmol), and triethylamine (0.022 mL, 0.156 mmol) in DMF (1 mL) was treated with HATU (13.1 mg, 0.034 mmol). The reaction was stirred at rt for 2 h. The reaction mixture was purified via preparative LC/MS to yield the title compound. LC-MS Method A: RT=1.99 min, [M+H]⁺=555.0; LC-MS Method B: RT=1.66 min, [M+H]⁺=555.1; ¹H NMR (500 MHz, DMSO-d₆) δ 8.46 (br dd, J=7.2, 3.8 Hz, 1H), 8.18-8.05 (m, 2H), 7.93 (s, 1H), 7.73 (d, J=4.0 Hz, 1H), 7.19 (d, J=4.0 Hz, 1H), 6.88 (br d, J=2.1 Hz, 1H), 6.86-6.64 (m, 1H), 5.29-5.01 (m, 2H), 4.87-4.54 (m, 2H), 4.13 (br d, J=4.3 Hz, 1H), 3.25-2.98 (m, 1H), 2.79-2.60 (m, 2H), 2.03-1.73 (m, 3H), 1.60-1.41 (m, 1H).

The following Examples in Table 63 were prepared using the procedure as shown in Example 880, replacing thiazole-2-carboxylic acid with the appropriate carboxylic acid. Various coupling reagents could be used other than the one described, such as BOP, PyBop, EDC/HOBt or T3P. In cases where the final product contains a basic primary or secondary amine, a Boc-protected amino acid was coupled, and the coupling step was followed by HCl or TFA-mediated cleavage of the Hoc-group.

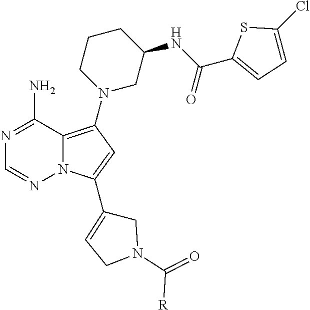

TABLE 63

| | | | | HPLC | |
|---|---|---|---|---|---|
| | | | LCMS | Method, | |
| Example | R | Name | (M + H)⁺ | RT (min.) | ¹H NMR |
| 881 | 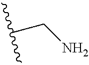 [SMILES: *CN] | (R)-N-(1-(4-amino-7-(1- glycyl-2,5-dihydro-1H- pyrrol-3-y1)pyrrolo[2,1- f][1,2,4]triazin-5- yl)piperidin-3-y1)-5- chlorothiophene-2- carboxamide | 501.2 | A: 1.38 B: 1.18 | (500 MHz, DMSO-d₆) δ 8.49 (br d, J=7.6 Hz, 1H), 7.86 (d, J=1.5 Hz, 1H), 7.69 (br d, J=3.7 Hz, 1H), 7.17 (d, J=3.1 Hz, 1H), 6.83 - 6.76 (m, 1H), 6.75 - 6.69 (m, 1H), 4.69 - 4.49 (m, 2H), 4.46 - 4.27 (m, 2H), 4.20 - 4.01 (m, 1H), 3.20 - 3.10 (m, 1H), 3.06 - 2,94 (m, IH), 2.79 - 2.58 (m, 2H), 2.00 - 1.70 (m, 6H), 1.59 - 1.46 (m, 1H) |
| 882 | 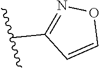 [SMILES: *c1ccon1] | (R)-N-(1-(4-amino-7-(1- (isoxazole-3-carbonyl)-2,5- dihydro-1H-pyrrol-3- yl)pyrrolo[2,1- f][1,2,4]triazin-5- y1)piperidin-3-y1)-5- chlorothiophene-2- carboxamide | 539.3 | A: 1.76 B: 1.53 | (500 MHz, DMSO-d₆) δ 9.10 (dd, J=7.2, 1.2 Hz, 1H), 8.56 - 8.41 (m, 1H), 7.96 (d, J-5.2 Hz, 1H), 7.70 (t, J=3.6 Hz, 1H), 7.17 (dd, J=3.6, 2.5 Hz, 1H), 6.93 (dd, J=2.8, 1.4 Hz, 1H), 6.87 (s, 1H), 6.83 (s, 1H), 6.73 (s, 1H), 4.96 (br s, 1H), 4.76 (br d, J=10.9 Hz, 2H), 4.54 (br d, J=2.2 Hz, 1H), 4.21 - 4.08 (m, 1H), 3.18 (brd, J=9.1 Hz, 1H), 3.09 - 3.00 (m, 1H), 2.76 - 2.62 (m, 2H), 1.99 - 1.77 (m, 3H), 1.58 - 1.43 (m, 1H). |
| 883 | 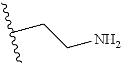 [SMILES: *CCN] | (R)-N-(1-(4-amino-7-(1-(3- aminopropanoy1)-2,5- dihydro-1H-pyrrol-3- yl)pyrrolo[2,1- f][1,2,4]triazin-5- y1)piperidin-3-y1)-5- chlorothiophene-2- carboxamide | 515.4 | A: 1.31 B: 1.17 | (500 MHz, DMSO-d₆) δ 8.51 (br dd, J=7.3, 3.4 Hz, 1H), 7.85 (s, 1H), 7.68 (br s, 1H), 7.15 (d, J-4.0 Hz, 1H), 6.80 - 6.75 (m, 1H), 6.70 (d, J=7.9 Hz, 1H), 4.68 (br s, 1H), 4.52 (br s, 1H), 4.45 (br s, 1H), 4.30 (br d, J=1.8 Hz, 1H), 4.15 - 4.02 (m, 1H), 3.20 - 3.12 (m, 1H), 3.05 - 2.96 (m, 1H), 2.93 (br d, J=4.0 Hz, 2H), 2.72 - 2.62 (m, 2H), 2.59 (br t, J=6.7 Hz, 1H), 1.97 - 1.82 (m, 2H), 1.58 - 1.42 (m), 1H). |
| 884 | 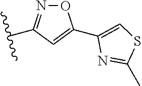 [SMILES: *c1cc(-c2csc(C)n2)on1] | (R)-N-(1-(4-amino-7-(1-(5- (2-methylthiazol-4- yl)isoxazole-3-carbony1)-2,5- dihydro-1H-pyrrol-3- yl)pyrrolo[2,1- f][1,2,4]triazin-5- y1)piperidin-3-y1)-5- chlorothiophene-2- carboxamide | 636.0 | A: 2.11 B: 1.75 | (500 MHz, DMSO-d₆) δ 8.55 - 8.39 (m, 1H), 8.29 (s, 1H), 7.95 (d, J=5.2 Hz, 1H), 7.72 (dd, J=7.2, 3.8 Hz, 1H), 7.22 - 7.17 (m, 1H), 7.12 (d. J=4.6 Hz, 1H), 6.87 (s, 1H), 6.85 - 6.70 (m, 1H), 4.99 (br s, 1H), 4.79 (s, 2H), 4.57 (br s, 1H), 4.22 - 4.07 (m, 1H), 3.26 - 3.14 (m, 1H), 3.10 - 2.99 (m, 1H), 2.79 - 2.64 (m, 5H), 1.98 - 1.77 (m, 3H), 1.59 - 1.43 (m, 1H). |
| 885 | 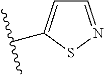 [SMILES: *c1ccns1] | (R)-N-(1-(4-amino-7-(1- (isothiazole-5-carbonyl)-2,5- dihydro-1H-pyrrol-3- yl)pyrrolo[2,1- f][1,2,4]triazin-5- y1)piperidin-3-y1)-5- chlorothiophene-2- carboxamide | 555.5 | A: 1.76 B: 1.50 | (500 MHz, DMSO-d₆) δ 8.73 (dd, J=18.0, 1.2 Hz, 1H), 8.55 - 8.38 (m, 1H), 8.10 - 7.85 (m, 2H), 7.73 (br d, J-4.0 Hz, 1H), 7.20 (br d, J = 4.0 Hz, 1H), 6.98 - 6.82 (m, 2H), 4.99 (br d, J=1.2 Hz, 1H), 4.79 (s, 2H), 4.56 (br d, J=1.8 Hz, 1H), 4.22 - 4.08 (m, 1H), 3.28 - 3.15 (m, IH), 3.12 - 3.01 (m, IH), 2.77 - 2.62 (m, 2H), 2.00 - 1.79 (m, 3H), 1.52 (br dd, J=7.8, 2.0 Hz, 1H). |
| 886 | 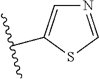 [SMILES: *c1cncs1] | (R)-N-(1-(4-amino-7-(1- (thiazole-5-carbonyl)-2,5- dihydro-1H-pyrrol-3- yl)pyrrolo[2,1- f][1,2,4]triazin-5- yl)piperidin-3-y1)-5- chlorothiophene-2- carboxamide | 555.4 | A: 1.64 B: 1.40 | (500 MHz, DMSO-d₆) δ 9.30 (d, J=5.5 Hz, 1H), 8.65 (s, 1H), 8.54 - 8.43 (m, 2H), 7.99 (s, 1H), 7.73 (d, J=3.7 Hz, IH), 7.19 (d, J=4.0 Hz, IH), 7.02 - 6.85 (m, 2H), 5.02 (br s, IH), 4.89 - 4.74 (m, 2H), 4.55 (br d, J=0.6 Hz, IH), 4.24 - 4.09 (m, 1H), 3,21 (br d, J-2.1 Hz, 1H), 3.05 (br d, J=10.7 Hz, IH), 2.69 (br d, J-7.9 Hz, 2H), 1.97 - 1.81 (m, 3H), 1.62 - 1.46 (m, 1H). |
| 887 | 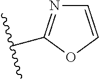 [SMILES: *c1ncco1] | (R)-N-(1-(4-amino-7-(1- (oxazole-2-carbonyl)-2,5- dihydro-1H-pyrrol-3- y1)pyrrolo[2,1- f][1,2,4]triazin-5- y1)piperidin-3-y1)-5- chlorothiophene-2- carboxamide | 539.1 | A: 1.78 B: 1.48 | (500 MHz, DMSO-d₆) δ 8.50 - 8.42 (m, 1H), 8.36 (d, J=7.3 Hz, IH), 7.92 (s, 1H), 7.72 (br d. J=4.0 Hz, 1H), 7.54 (d. J=18.0 Hz, 1H), 7.19 (d, J-4.0 Hz, 1H), 6.86 (br d, J-8.5 Hz, 1H), 6.84 - 6.64 (m, 1H), 5.16 (br s, 1H), 4.95 (br s, 1H), 4.78 (br s, IH), 4.55 (br s, IH), 4.19 - 4.07 (m, 1H), 3.39 - 3.13 (m, 1H), 3.08 - 2.93 (m, 1H), 2.75 - 2.62 (m, 2H), 2.00 - 1.77 (m, 3H), 1.62 - 1,46 (m, 1H). |
| 888 | 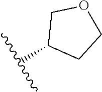 [SMILES: *[C@H]1CCOC1] | N-((R)-1-(4-amino-7-(1-((S)- tetrahydrofuran-3-carbonyl)- 2,5-dihydro-1H-pyrrol-3- yl)pyrrolo[2,1- f][1,2,4]triazin-5- yl)piperidin-3-y1)-5- chlorothiophene-2- carboxamide | 542.2 | A: 1.65 B: 1.39 | (500 MHz, DMSO-d₆) δ 8.47 (br dd, J=7.3, 4.0 Hz, 1H), 8.05 - 7.84 (m, 2H), 7.77 - 7.65 (m, IH), 7.25 - 7.15 (m, IH), 7.04 - 6.65 (m, 3H), 4.82 - 4.69 (m, 1H), 4.53 (br s, 2H), 4.31 (br s, IH), 4.10 (br d, J=4.3 Hz, 1H), 4.02 - 3.87 (m, 1H), 3.83 - 3.67 (m, 2H), 3.43 - 2.95 (m, 2H), 2.81 - 2.60 (m, 2H), 2.21 - 2.07 (m, 1H), 2.06 - 1.97 (m, IH), 1.96 - 1.74 (m, 3H), 1.59 - 1.44 (m, 1H). |
| 889 | 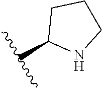 [SMILES: *[C@H]1CCCN1] | N-((R)-1-(7-(1-(D-proly1)- 2,5-dihydro-1H-pyrrol-3-y1)- 4-aminopyrrolo[2,1- f][1,2,4]triazin-5- yl)piperidin-3-y1)-5- chlorothiopbene-2- carboxamide, TFA | 541.5 | A: 1.37 B: 1.17 | (500 MHZ, DMSO-d₆) δ 9.65 - 9.37 (m, IH), 8.70 - 8.55 (m, 1H), 8.49 (br t, J-7.8 Hz, IH), 8.14 - 7.95 (m, 1H), 7.75 - 7.68 (m, IH), 7.30 - 7.05 (m, 5H), 6.86 - 6.71 (m, 2H), 4.87 - 4.31 (m, 5H), 4.12 (br d, J-4.0 Hz, 1H), 3.39 - 3.13 (m, 2H), 3.09 - 2.96 (m, 1H), 2.76- 2.61 (m, 2H), 2.05 - 1.77 (m, 6H), 1.64 - 1.45 (m, 1H). |
| 890 | 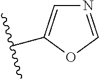 [SMILES: *c1cnco1] | (R)-N-(1-(4-amino-7-(1- (oxazole-5-carbony1)-2,5- dihydro-1H-pyrrol-3- y1)pyrrolo[2,1- f][1,2,4]triazin-5- yl)piperidin-3-y1)-5- chlorothiophene-2- carboxamide | 539.4 | A: 1.56 B: 1.34 | (500 MHZ, DMSO-d₆) δ 8.63 (br d, J=5.5 Hz, IH), 8.53 - 8.40 (m, 1H), 8.10 - 7.85 (m, 2H), 7.73 (br s, 1H), 7.19 (br d, J=3.7 Hz, IH), 6.99 - 6.81 (m, 2H), 4.95 (br d, J=1.2 Hz, 1H), 4.78 (br d, J= 13.4 Hz, 2H), 4.53 (br s, 1H), 4.24 - 4.11 (m, IH), 3.22 (br s, 1H), 3.04 (br s, 1H), 2.79 - 2.60 (m, 2H), 1.99 - 1.77 (m, 3H), 1.60 - 1.44 (m, 1H) |
| 891 | 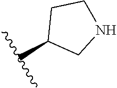 [SMILES: *[C@H]1CCNC1] | N-((R)-1-(4-amino-7-(1-((S)- pyrrolidine-3-carbonyl)-2,5- dihydro-1H-pyrrol-3- y1)pyrrolo[2,1- f][1,2,4]triazin-5- y1)piperidin-3-y1)-5- chlorothiophene-2- carboxamide | 541.1 | A: 1.30 B: 1.19 | (500 MHZ, DMSO-d₆) δ 8.50 - 8.41 (m, 1H), 7.88 (s, 1H), 7.72 (br s, 1H), 7.19 (dd, J=4.0, 2.1 Hz, 1H), 6.80 (br d, J=4.9 Hz, 1H), 6.76- 6.69 (m, 1H), 4.78 (br s, 1H), 4.63 - 4.50 (m, 2H), 4.33 (br s, IH), 4.20 - 4.04 (m, 1H), 3.91 (s, IH), 3.23 - 3.09 (m, 2H), 3.08 - 2.95 (m, 1H), 2.77 - 2.59 (m, 2H), 2.35 - 2.09 (m, 2H), 2.05 - 1,74 (m, 6H), 1.61 - 1.44 (m, 2H). |
| 892 | 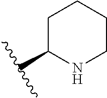 [SMILES: *[C@H]1CCCCN1] | N-((R)-1-(4-amino-7-(1-((R)- piperidine-2-carbonyl)-2,5- dihydro-1H-pyrrol-3- y1)pyrrolo[2,1- f][1,2,4]triazin-5- yl)piperidin-3-y1)-5- chlorothiophene-2- carboxamide, TFA | 555.1 | A: 1.42 B: 1.22 | (500 MHz, DMSO-d₆) δ 9.06 - 8.92 (m, IH), 8.82 - 8.64 (m, 1H), 8.49 (br dd, J=11.1, 7.8 Hz, 1H), 7.97 (s, IH), 7.76 - 7.69 (m, 1H), 7.28 - 7.04 (m, 2H), 6.87 (s, IH), 6.84 - 6.70 (m, 1H), 4.89 - 4.70 (m, 1H), 4.70 - 4.61 (m, 1H), 4.60 - 4.51 (m, 1H), 4.51 - 4.30 (m, 1H), 4.24 - 4.08 (m, 2H), 3.33 - 3.15 (m, 1H), 3.12 - 2.88 (m, 2H), 2.74 - 2.61 (m, 2H), 2.26 - 2.12 (m, 1H), 1.99 - 1.46 (m, 9H). |
| 893 | 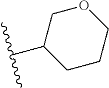 [SMILES: *C1CCCOC1] | N-((3R)-1-(4-amino-7-(1- (tetrahydro-2H-pyran-3- carbony1)-2,5-dihydro-1H- pyrrol-3-yl)pyrrolo[2,1- f][1,2,4]triazin-5- y1)piperidin-3-y1)-5- chlorothiophene-2- carboxamide | 556.2 | A: 1.74 B: 1.46 | (500 MHz, DMSO-d₆) δ 8.46 (br t, J=6.6 Hz, 1H), 8.04 - 7.82 (m, 2H), 7.72 (br t, J=3.7 Hz, 1H), 7.18 (dd, J=3.8, 2.6 Hz, IH), 7.02 - 6.82 (m, 1H), 6.81 - 6.70 (m, 2H), 4.89 - 4.71 (m, 1H), 4.64 - 4.40 (m, 2H), 4.27 (br s, 1H), 4.17 - 4.04 (m, 1H), 3.99 - 3.77 (m, 2H), 3.42 - 3.10 (m, 1H), 3.06 - 2.92 (m, 1H), 2.87 - 2.59 (m, 3H), 2.04 - 1.34 (m, 9H). |
| 894 | 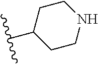 [SMILES: *C1CCNCC1] | (R)-N-(1-(4-amino-7-(1- (piperidine-4-carbonyl)-2,5- dihydro-1H-pyrrol-3- yl)pyrrolo[2,1- f][1,2,4]triazin-5- yl)piperidin-3-y1)-5- chlorothiophene-2- carboxamide | 555.6 | A: 1.32 B: 1.16 | (500 MHZ, DMSO-d₆) δ 8.47 (br t, J=6.6 Hz, 1H), 7.87 (d, J= 4.3 Hz, IH), 7.72 (br t, J=4.0 Hz, IH), 7.18 (t, J=3.5 Hz, IH), 6.80 (br d, J=15.9 Hz, IH), 6.73 - 6.65 (m, IH), 4.76 (br s, 1H), 4.62 - 4.46 (m, 2H), 4.29 (br s, 1H), 4.16 - 4.04 (m, 1H), 3.29 - 3.11 (m, 2H), 3.08 - 2.92 (m, 1H), 2.89 - 2.77 (m, 2H), 2.76 - 2.59 (m, 2H), 1.98 -1.76 (m, 6H), 1.75 - 1.62 (m, 2H), 1.59 - 1.48 (m, 1H). |
| 895 | 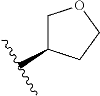 [SMILES: *[C@@H]1CCOC1] | N-((R)-1-(4-amino-7-(1-((R)- tetrahydrofuran-3-carbonyl)- 2,5-dihydro-1H-pyrrol-3- yl)pyrrolo[2,1- f][1,2,4]triazin-5- yl)piperidin-3-y1)-5~ chlorothiophene-2- carboxamide | | A: 1.65 B: 1.39 | (500 MHz, DMSO-d₆) δ 8.47 (br dd, J = 7.2, 4.1 Hz, 1H), 8.01-7.89 (m, 1H), 7.87 (d, J=2.7 Hz, 1H), 7.75 - 7.67 (m, 1H), 7.18 (dd, J-3.7, 1.8 Hz, 1H), 6.96 - 6.83 (m, 1H), 6.79 (br d, J=15.6 Hz, IH), 6.73 (d, J=3.1 Hz, IH), 4.75 (br d, J-1.2 Hz, IH), 4.62 - 4.45 (m, 2H), 4.31 (br s, 1H), 4.18 - 4.06 (m, 1H), 4.02 - 3.88 (m, 1H), 3.83 - 3.66 (m, 2H), 3.43 - 3.12 (m, 2H), 3.09 - 2.96 (m, 1H), 2.76 - 2.58 (m, 2H), 2.20 - 2.07 (m, 1H), 2.06 - 1.97 (m, IH), 1.97 - 1.73 (m, 3H), 1.58 - 1.44 (m, 1H).542.1 |
| 896 | 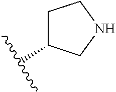 [SMILES: *[C@@H]1CCNC1] | N-((R)-1-(4-amino-7-(1-((R)- pyrrolidine-3-carbonyl)-2,5- dihydro-1H-pyrrol-3- yl)pyrrolo[2,1- f][1,2,4]triazin-5- yl)piperidin-3-y1)-5- chlorothiophene-2- carboxamide, TFA | 541.5 | A: 1.32 B: 1.15 | (500 MHz, DMSO-d₆) δ 9.06 - 8.85 (m, 2H), 8.48 (br t, J=7.6 Hz, 1H), 7.94 (d. J=2.1 Hz, IH), 7.73 (t, J=3.1 Hz, 1H), 7.31 - 7.05 (m, 2H), 6.88 - 6.71 (m, 2H), 4.89 - 4.71 (m, 1H), 4,67 - 4.52 (m, 2H), 4.34 (br s, 1H), 4.23 - 4.06 (m, IH), 3.48 - 3.33 (m, 1H), 3.30 - 3.15 (m, 2H), 3.08 - 2.95 (m, IH), 2.76 - 2.60 (m, 2H), 2.37 - 2.21 (m, 1H), 2.08 - 1.77 (m, 4H), 1.63 - 1.45 (m, 1H). |
| 897 | 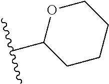 [SMILES: *C1CCCCO1] | N-((3R)-1-(4-amino-7-(1- (tetrahydro-2H-pyran-2- carbony1)-2,5-dihydro-1H- pyrrol-3-y1)pyrrolo[2,1- f][1,2,4]triazin-5- yl)piperidin-3-y1)-5- chlorothiophene-2- carboxamide | 556.2 | A: 1.81 B: 1.53 | (500 MHz, DMSO-d₆) δ 8.52-8.45 (m, IH), 7.98 (s, 1H), 7.75-7.68 (m, 1H), 7.20-7.17 (m, 1H), 6.84 (s, 1H), 6.82-6.76 (m, 1H), 4.85- 4.71 (m, 1H), 4.67-4.48 (m, 2H), 4.31 (br s, 1H), 4.22-4.04 (m, 2H), 3.98-3.87 (m, 1H), 3.27 - 3.15 (m, 1H), 3.04 (br dd, J=7.0, 2.4 Hz, IH), 2.78 - 2.61 (m, 2H), 2.00 - 1.75 (m, 4H), 1.73-1.42 (m, 7H) |
| 898 | 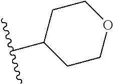 [SMILES: *C1CCOCC1] | (R)-N-(1-(4-amino-7-(1- (tetrahydro-2H-pyran-4- carbony1)-2,5-dihydro-1H- pyrrol-3-yl)pyrrolo[2,1- f][1,2,4]triazin-5- y1)piperidin-3-y1)-5- chlorothiophene-2- carboxamide | 556.1 | A: 1.67 B: 1.41 | (500 MHz, DMSO-d₆) δ 8.47 (br t, J=6.6 Hz, 1H), 8.01 - 7.84 (m, 2H), 7.72 (br t, J=3.8 Hz, IH), 7.18 (t, J=3.7 Hz, IH), 6.98 - 6.76 (m, 2H), 6.72 (s, 1H), 4.76 (br s, IH), 4.57 (brs, 1H), 4.51 (br s, IH), 4.29 (br s, IH), 4.20 - 4.05 (m, 1H), 3.96 - 3.85 (m, 2H), 3.22-3.12 (m, 1H), 3.07 - 2.93 (m, IH), 2.89 - 2.78 (m, IH), 2.77 - 2.60 (m, 2H), 2.02 - 1.73 (m, 3H), 1.63 (brd, J=4.0 Hz, 4H), 1.56 - 1.41 (m, 1H). |
| 899 | 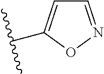 [SMILES: *c1ccno1] | (R)-N-(1-(4-amino-7-(1- (isoxazole-5-carbony1)-2,5- dihydro-1H-pyrrol-3- y1)pyrrolo[2,1- f][1,2,4]triazin-5- y1)piperidin-3-y1)-5- chlorothiophene-2- carboxamide | 539.1 | A: 1.75 B: 1.48 | (500 MHZ, DMSO-d₆) δ 8.82 (dd, J=15.3, 1.5 Hz, 1H), 8.54 - 8.42 (m, 1H), 8.12 - 7.94 (m, 1H), 7.90 (s, 1H), 7.73 (br d, J-3.1 Hz, 1H), 7.30 - 7.12 (m, 2H), 7.01 - 6.76 (m, 3H), 5.00 (br s, IH), 4,79 (br d, J=14.0 Hz, 2H), 4.55 (br s, IH), 4.24 - 4.02 (m, IH), 2.78 - 2.59 (m, 2H), 1.94 - 1.77 (m, 3H), 1.55 - 1.45 (m, 1H). |
| 900 | 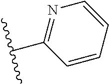 [SMILES: *c1ccccn1] | (R)-N-(1-(4-amino-7-(1- picolinoy1-2,5-dihydro-1H- pyrrol-3-yl)pyrrolo[2,1- f][1,2,4]triazin-5- y1)piperidin-3-y1)-5- chlorothiophene-2- carboxamide | 549.5 | A: 1.71 B: 1.45 | (500 MHz, DMSO-d₆) δ 8.74 - 8.61 (m, 1H), 8.45 (br dd, J=19.4, 7.8 Hz, IH), 8.01 - 7.95 (m, IH), 7.92 - 7.81 (m, 2H), 7.72 (br dd, J=8.7, 3.8 Hz, 1H), 7.62 - 7.46 (m, 1H), 7.19 (t, J=4.0 Hz, 1H), 6.92- 6.57 (m, 2H), 4.94 (br s, 1H), 4.82 - 4.68 (m, 2H), 4.57 (br s, 1H), 4.26 - 4.03 (m, 1H), 3.23 - 2.94 (m, 1H), 2.80 - 2.58 (m, 1H), 2.00 - 1.73 (m, 3H), 1.63 - 1.36 (m, 1H). |
| 901 | 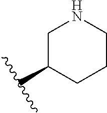 [SMILES: *[C@@H]1CCCNC1] | N-((R)-1-(4-amino-7-(1-((R)- piperidine-3-carbonyl)-2,5- dihydro-1H-pyrrol-3- y1)pyrrolo[2,1- f][1,2,4]triazin-5- y1)piperidin-3-y1)-5- chlorothiophene-2- carboxamide | 555.2 | A: 1.34 B: 1.22 | (500 MHZ, DMSO-d₆) δ 8.52 - 8.42 (m, 1H), 7.89 - 7.83 (m, 1H), 7.75 - 7.68 (m, 1H), 7.21 - 7.16 (m, 1H), 6.79 (br d, J-6.4 Hz, 1H), 6.72 (s, 1H), 4.76 (br s, 1H), 4.54 (brs, 1H), 4.50 (br s, 1H), 4.28 (br s, IH), 4.16 - 4.02 (m, 1H), 3.23 - 3.11 (m, 1H), 3.10 - 2.89 (m, 2H), 2.77 - 2.59 (m, 3H), 1.97 - 1.76 (m, 8H), 1.70 - 1.61 (m, 1H), 1.60 - 1.41 (m, 3H). |
| 902 | 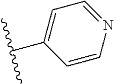 [SMILES: *c1ccncc1] | (R)-N-(1-(4-amino-7-(1- isonicotinoy1-2,5-dihydro- 1H-pyrrol-3-yl)pyrrolo[2,1- f][1,2,4]triazin-5- yl)piperidin-3-y1)-5- chlorothiophene-2- carboxamide | 549.5 | A: 1.56 B: 1.23 | (500 MHz, DMSO-d₆) δ 8.76 (br dd, J=10.7, 5.5 Hz, 2H), 8.54-8.40 (m, 1H), 7.99 (d. J=14.3 Hz, IH), 7.71 (dd, J=12.4, 3.8 Hz, IH), 7.66 (br dd, J=15.7, 5.6 Hz, 2H), 7.22 - 7.17 (m, 1H), 6.92 (s, 1H), 6.81 - 6.66 (m, IH), 4.78 (br s, 1H), 4.64 (br s, 1H), 4.55 (br s, IH), 4.42 (br s, 1H), 4.23 - 4.05 (m, 1H), 3,26 - 3.12 (m, 1H), 3.11 - 2.95 (m, 1H), 2.77 - 2.58 (m, 2H), 1.97 - 1.78 (m, 3H), 1.60 - 1.39 (m, 1H) |
| 903 | 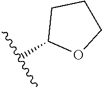 [SMILES: *[C@@H]1CCCO1] | N-((R)-1-(4-amino-7-(1-((S)- tetrahydrofuran-2-carbony1)- 2,5-dihydro-1H-pyrrol-3- yl)pyrrolo[2,1- f][1,2,4]triazin-5- yl)piperidin-3-y1)-5- chlorothiophene-2- carboxamide | 542.6 | A: 1.62 B: 1.39 | (500 MHz, DMSO-d₆) δ 8.45 (br d, J=7.0 Hz, IH), 8.07 - 7.91 (m, 1H), 7.88 (s, 1H), 7.72 (br d, J=2.7 Hz, IH), 7.22 - 7.14 (m, 1H), 6.97 - 6.84 (m, 1H), 6.79 (br d, J=12.8 Hz, IH), 6.76 - 6.69 (m, 1H), 4.88 - 4.68 (m, 1H), 4.66 - 4.56 (m, 1H), 4.55 - 4.49 (m, 1H), 4.31 (br d, J=1.5 Hz, 1H), 4.18 - 4.05 (m, 1H), 3.89 - 3.74 (m, 2H), 3.21- 2.99 (m, 1H), 2.76 - 2.59 (m, 1H), 2.16 - 2.00 (m, 2H), 1.98 - 1.72 (m, 5H), 1.58 - 1.43 (m, 1H). |
| 904 | 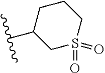 [SMILES: *C1CCCS(=O)(=O)C1] | N-((3R)-1-(4-amino-7-(1- (1,1-dioxidotetrahydro-2H- thiopyran-3-carbonyl)-2,5- dihydro-1H-pyrrol-3- yl)pyrrolo[2,1- f][1,2,4]triazin-5- y1)piperidin-3-y1)-5- chlorothiophene-2- carboxamide | 604.1 | A: 1.63 B: 1.38 | (500 MHZ, DMSO-d₆) δ 8.45 (br d, J=7.6 Hz, IH), 8.07 - 7.93 (m, IH), 7.88 (d. J=4.6 Hz, IH), 7.72 (br t, J=3.7 Hz, IH), 7.19 (br d, J=2.4 Hz, 1H), 7.01 - 6.85 (m, 1H), 6.85 - 6.74 (m, 2H), 4.86 - 4.72 (m, IH), 4.66 - 4.45 (m, 2H), 4.30 (br s, IH), 4.17 - 4.05 (m, 1H), 3.24 - 3.00 (m, 3H), 2.76 - 2.59 (m, 2H), 2.16 - 2.07 (m, 1H), 2.04 - 1.73 (m, 5H), 1.65 - 1.42 (m, 2H). |
| 905 | 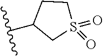 [SMILES: *C1CCS(=O)(=O)C1] | N-((3R)-1-(4-amino-7-(1- (1,1- dioxidotetrahydrothiophene- 3-carbonyl)-2,5-dihydro-1H- pyrrol-3-yl)pyrrolo[2,1- f][1,2,4]triazin-5- yl)piperidin-3-y1)-5- chlorothiophene-2- carboxamide | 590.0 | A: 1.61 B: 1.37 | (500 MHZ, DMSO-d₆) δ 8.52 - 8.41 (m, 1H), 7.98 (s, 1H), 7.72 (br d, J=1.8 Hz, 1H), 7.20 (br s, 1H), 6.90 - 6.71 (m, 2H), 4.90 - 4.72 (m, IH), 4.65 - 4.54 (m, 1H), 4.35 (br s, 1H), 4.25 - 4.08 (m, 1H), 3.49 - 3.00 (m, 5H), 2.78 - 2.60 (m, 2H), 2.49 - 2.37 (m, 1H), 2.23 - 2.06 (m, IH), 2.01 - 1.77 (m, 3H), 1.52 (br dd, J=7.0, 3.4 Hz, 1H). |

The following Examples in Table 64 were prepared using the procedure as shown in Example 880, replacing (R)—N-(1-(4-amino-7-(2,5-dihydro-1H-pyrrol-3-yl)pyrrolo[2,1-f][1,2,4]triazin-1-yl)piperidin-3-yl)-5-chlorothiophene-2-carboxamide hydrochloride with Example 171, and thiazole-2-carboxylic acid with the appropriate carboxylic acid. Various coupling reagents could be used other than the one described, such as BOP, PyBop. EDC/HOBt or T3P.

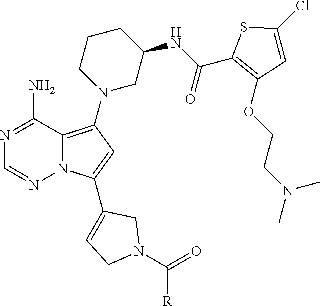

TABLE 64

| | | | | HPLC | |
|---|---|---|---|---|---|
| | | | | Method, | |
| | | | LCMS | RT | |
| Example | R | Name | (M+H) | (min.) | IHNMR |
| 906 | N-O | (R)-N-(1-(4-amino-7-(1- | 648.0 | A: 1.78 | (500 MHz, DMSO-d6) 8 9.16 - 9.09 (m, IH), 8.13 - 7.96 (m, IH), |
| | | (isoxazole-3-carbonyl)- | | B: 1.20 | 7.88 (d, J=2.7 Hz, 1H), 7.78 - 7.65 (m, IH), 7.35 (d, J=1.8 Hz, 1H), |
| | | 2,5-dihydro-1H-pyrrol-3- | | | 6.94 (s, 1H), 6.89 - 6.62 (m, 2H), 4.95 (br s, 1H), 4.76 (br d, J=6.1 |
| | | yl)pyrrolo[2,1- | | | Hz, 2H), 4.55 (br s, 1H), 4.29 (br s, 2H), 4.20 - 4.08 (m, 1H), 3.91 (s, |
| | | f\|[1,2,4]triazin-5- | | | 1H), 3.21 - 3.08 (m, IH), 3.05 - 2.93 (m, 1H), 2.65 - 2.56 (m, 2H), |
| | | yl)piperidin-3-y1)-5- | | | 2.18 (br d, J=4.3 Hz, 6H), 1.98 - 1.78 (m, 3H), 1.57 - 1.34 (m, IH). |
| | | chloro-3-(2- | | | |
| | | (dimethylamino)ethoxy)t | | | |
| | | hiophene-2-carboxamide | | | |
| Example | R | Name | LCMS | HPLC | IHNMR |
| | | | (M+H) | Method, | |
| | | | + | RT | |
| | | | | (min.) | |
| 907 | | (R)-N-(1-(4-amino-7-(1- | 626.1 | A: 1.67 | (500 MHz, DMSO-d6) o 8.61 (d, J=6.7 Hz, IH), 8.10 - 7.83 (m, 3H), |
| | | (oxazole-5-carbonyl)- | | B: 1.01 | 7.73 (br d, J=5.2 Hz, IH), 7.34 (s, 1H), 6.89 (s, IH), 6.81 (brs, IH), |
| | | 2,5-dihydro-1H-pyrrol-3- | | | 4.93 (br s, IH), 4.76 (br d, J=16.5 Hz, 2H), 4.51 (br d, J=1.5 Hz, |
| | | yl)pyrrolo[2,1- | | | IH), 4.35 - 4.24 (m, 2H), 4.14 (br s, IH), 3.06 - 2.92 (m, 1H), 2.71 - |
| | | f][1,2,4]triazin-5- | | | 2.56 (m, 2H), 2.17 (br s, 6H), 2.01 - 1.74 (m, 3H), 1.59 - 1.38 (m, |
| | | yI)piperidin~3-y1)-5- | | | 1H). |
| | | chloro-3-(2- | | | |
| | | (dimethylamino)ethoxy)t | | | |
| | | hiophene-2-carboxamide | | | |
| 908 | | (R)-N-(1-(4-amino-7-(1- | 626.0 | A: 1.84 | (500 MHz, DMSO-d6) 8 8.34 (d, J=7.3 Hz, 1H), 7.88 (s, 1H), 7.79 - |
| | | (oxazole-2-carbonyl)- | | B: 1.26 | 7.68 (m, IH), 7.53 (d, J=14.6 Hz, IH), 7.33 (s, IH), 6.84 (br d, |
| | | 2,5-dihydro-1H-pyrrol-3- | | | J=10.7 Hz, 1H), 6.81 - 6.61 (m, IH), 5.14 (br s, IH), 4.93 (br s, 1H), |
| | | yl)pyrrolo[2,1- | | | 4.76 (br s, IH), 4.54 (br s, IH), 4.28 (brt, J=5.0 Hz, 2H), 4.20 ~ 4.09 |
| | | f][1,2,4]triazin-5- | | | (m, 1H), 3.02 - 2.91 (m, IH), 2.87 - 2.68 (m, IH), 2.65 - 2.56 (m, |
| | | yl)piperidin-3-y1)-5- | | | 2H), 2.17 (br s, 6H), 1.96 - 1.74 (m, 3H), 1.55 - 1.39 (m, 1H). |
| | | chloro-3-(2- | | | |
| | | (dimethylamino)ethoxy)t | | | |
| | | hiophene-2-carboxamide | | | |
| 909 | | N-((3R)-1-(4-amino-7- | 643.2 | A: 1.66 | (500 MHz, DMSO-d6) 8 7.87 (d, J=4.0 Hz, IH), 7.71 (br d, J=6.7 |
| | | (1-(tetrahydro-2H-pyran- | | B: 1.16 | Hz, IH), 7.36 (d, J=2.7 Hz, 1H), 6.84 - 6.72 (m, 2H), 4.78 (br d, |
| | | 3-carbonyl)-2,5-dihydro- | | | J=1.5 Hz, IH), 4.58 (br s, 1H), 4.48 (br s, 1H), 4.28 (br dd, J=9.6, |
| | | 1H-pyrrol-3~ | | | 3.8 Hz, 3H), 4.20 - 4.11 (m, 1H), 3,99 - 3.90 (m, 1H), 3.84 (brt, J=9,6 |
| | | yl)pyrrolo[2,1- | | | Hz, IH), 3.02 - 2.92 (m, IH), 2.88 - 2.78 (m, 1H), 2.77 - 2.67 (m, |
| | | f][1,2,4]triazin-5- | | | 1H), 2.65 ~ 2.56 (m, 2H), 2.18 (br s, 6H), 1.97 - 1.75 (m, 5H), 1.73 - |
| | | y1)piperidin-3-y1)-5- | | | 1.40 (m, 5H). |
| | | chloro-3-(2- | | | |
| | | (dimethylamino)ethoxy)t | | | |
| | | hiophene-2-carboxamide | | | |

### Example 910

### Preparation of (R)-5-(1-acetyl-2,5-dihydro-1H-pyrrol-3-yl)-N-(1-(7-(1-acetyl-2,5-dihydro-1H-pyrrol-3-yl)-4-aminopyrrolo[2,1-f][1,2,4]triazin-5-yl)piperidin-3-yl)-3-(2-(dimethylamino)ethoxy)thiophene-2-carboxamide

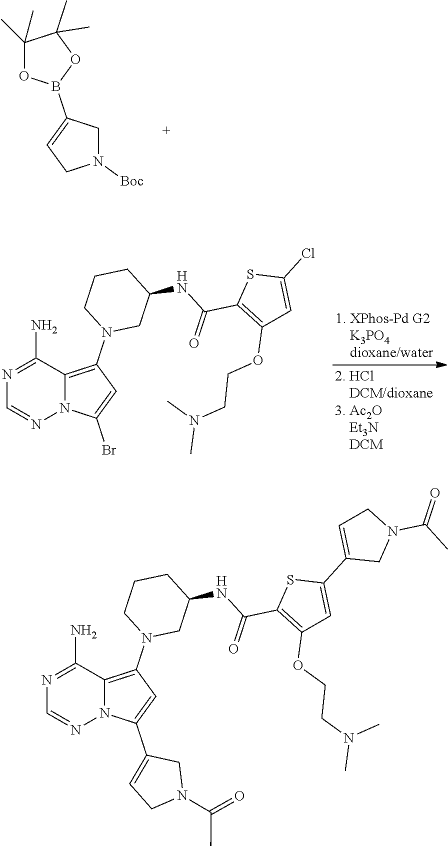 [SMILES: CC(C)(C)OC(=O)N1CC=C(B2OC(C)(C)C(C)(C)O2)C1 ; CN(C)CCOc1cc(Cl)sc1C(=O)N[C@@H]1CCCN(c2cc(Br)n3ncnc(N)c23)C1 ; CC(=O)N1CC=C(c2cc(OCCN(C)C)c(C(=O)N[C@@H]3CCCN(c4cc(C5=CCN(C(C)=O)C5)n5ncnc(N)c45)C3)s2)C1]

A mixture of (R)—N-(1-(4-amino-7-bromopyrrolo[2,1-f][1,2,4]triazin-5-yl)piperidin-3-yl)-5-chloro-3-(2-(dimethylamino)ethoxy)thiophene-2-carboxamide (247 mg, 0.455 mmol), tert-butyl 3-(4,4,5,5-tetramethyl-1,3,2-dioxaborolan-2-yl)-2,5-dihydro-1H-pyrrole-1-carboxylate (161 mg, 0.546 mmol), potassium phosphate, tribasic (2 M) (0.682 mL, 1.365 mmol), and 2nd generation XPhos precatalyst (17.5 mg, 0.023 mmol) in dioxane (5 mL) was placed in a sonicator and degassed with bubbling nitrogen for 5 minutes. The vial was sealed, and the reaction was stirred at 40° C. for 30 minutes. The mixture was diluted with ethyl acetate (5 mL), and the turbid solution was washed with water (2×) and brine. The organic phase was dried over sodium sulfate and concentrated in vacuo. The residue was purified by flash chromatography (0% to 18% methanol/dichloromethane gradient) The product was taken up in dichloromethane (5 mL) and was treated with 4M HCl in dioxane. The reaction was stirred at rt for 1 h, then was concentrated. The crude material was taken up in dichloromethane (1 mL), then was treated with triethylamine (0.024 mL, 0.17 mmol) followed by acetic anhydride (5.3 μl, 0.056 mmol). The reaction was stirred at rt for 1 h, then was concentrated. The crude material was purified via preparative HPLC to yield the title compound (13.7 mg, 74% yield). LC-MS Method A: RT=1.33 min, [M+H]⁺=648.7; LC-MS Method B: RT=0.99 min, [M+H]⁺=648.7; ¹H NMR (500 MHz, DMSO-d₆) δ 8.08-7.92 (m, 1H), 7.87 (s, 1H), 7.77 (br d, J=6.1 Hz, 1H), 7.27 (br d, J=12.2 Hz, 1H), 6.92-6.64 (m, 3H), 6.34 (br d, J=1.2 Hz, 1H), 4.69 (br s, 1H), 4.60 (br s, 1H), 4.48 (br d, J=11.3 Hz, 2H), 4.41 (br d, J=11.0 Hz, 2H), 4.34-4.20 (m, 4H), 4.15 (br d, J=4.6 Hz, 1H), 3.45-3.32 (m, 1H), 3.04-2.91 (m, 1H), 2.85-2.58 (m, 3H), 2.18 (br s, 6H), 2.11-1.97 (m, 6H), 1.93-1.75 (m, 3H), 1.57-1.41 (m, 1H).

### Example 911

### Preparation of methyl (R)-2-(3-(4-amino-5-(3-(5-chlorothiophene-2-carboxamido)piperidin-1-yl)pyrrolo[2,1-f][1,2,4]triazin-7-yl)-2,5-dihydro-1H-pyrrol-1-yl)acetate

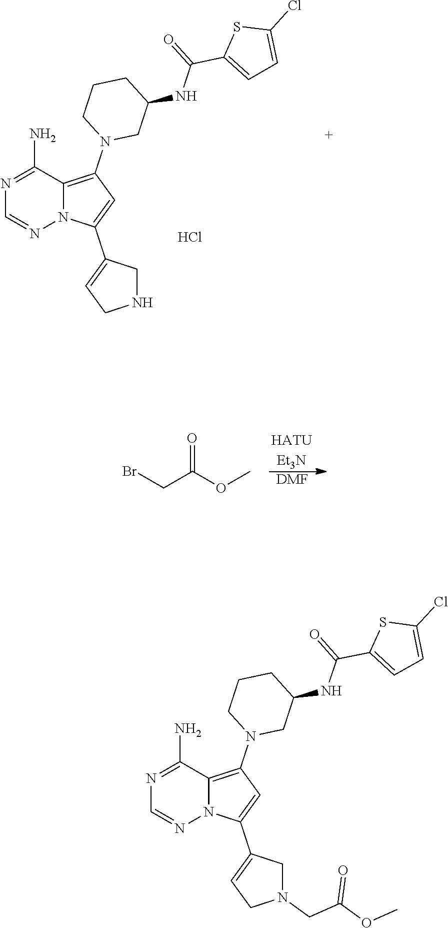

A stirring solution of (R)—N-(1-(4-amino-7-(2,5-dihydro-1H-pyrrol-3-yl)pyrrolo[2,1-f][1,2,4]triazin-5-yl)piperidin-3-yl)-5-chlorothiophene-2-carboxamide hydrochloride (30 mg, 0.062 mmol) and triethylamine (0.044 mL, 0.312 mmol) in dichloromethane (1 mL) was treated with methyl 2-bromoacetate (6.3 μl, 0.069 mmol). The reaction was stirred at rt for 6 h. The mixture was treated with methyl 2-bromoacetate (6.3 μl, 0.069 mmol), and the reaction was stirred at it for 18 h. The mixture was treated with methanol, then concentrated in vacuo. Half of the residue was purified via preparative HPLC to yield the title compound (6.0 mg, 18% yield). LC-MS Method A: RT=1.69 min, [M+H]⁺=516.1; LC-MS Method B: RT=1.17 min, [M+H]⁺=516.9; ¹H NMR (500 MHz, DMSO-d₆) δ 8.45 (d, J=7.7 Hz, 1H), 7.84-7.77 (m, 1H), 7.72-7.69 (m, 1H), 7.17 (d, J=4.1 Hz, 1H), 6.76-6.67 (m, 1H), 6.60-6.52 (m, 1H), 4.16-4.03 (m, 1H), 3.98 (br s, 2H), 3.83-3.74 (m, 2H), 3.67-3.53 (m, 3H), 3.14 (br dd, J=10.0, 2.0 Hz, 1H), 3.06-2.92 (m, 1H), 2.74-2.57 (m, 2H), 2.01-1.69 (m, 3H), 1.60-1.40 (m, 1H).

### Example 912

### Preparation of (R)-2-(3-(4-amino-5-(3-(5-chlorothiophene-2-carboxamido)piperidin-1-yl)pyrrolo[2,1-f][1,2,4]triazin-7-yl)-2,5-dihydro-1H-pyrrol-1-yl)acetic acid

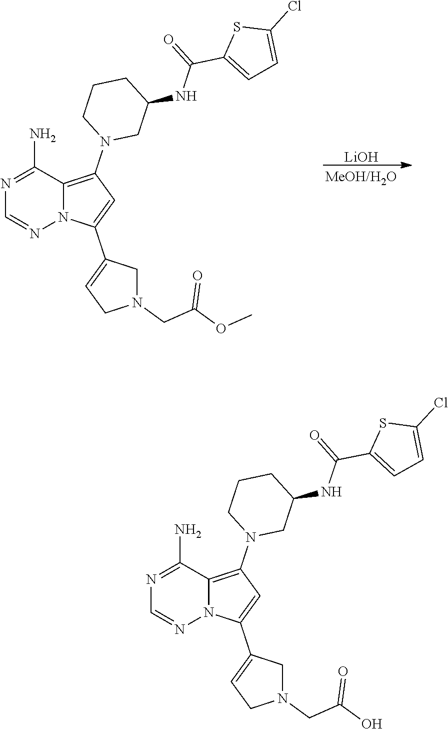 [SMILES: COC(=O)CN1CC=C(c2cc(N3CCC[C@@H](NC(=O)c4ccc(Cl)s4)C3)c3c(N)ncnn23)C1 ; "LiOH" ; Nc1ncnn2c(C3=CCN(CC(=O)O)C3)cc(N3CCC[C@@H](NC(=O)c4ccc(Cl)s4)C3)c12]

A suspension of Example 911 (16.0 mg, 0.031 mmol) in methanol (1 mL) was treated with 0.5 M LiOH (0.248 mL, 0.124 mmol). The reaction was stirred at 60° C. for 4 hours, then the pH was adjusted to 3 with 1M HCl, then concentrated in vacuo. The crude material was purified via preparative LC/MS to yield the title compound (4.5 mg, 28% yield). LC-MS Method A: RT=1.29 min, [M+H]⁺=502.2; LC-MS Method B: RT=1.09 min, [M+H]⁺=502.2; ¹H NMR (500 MHz, DMSO-d₆) δ 8.49 (br d, J=7.9 Hz, 1H), 7.88-7.75 (m, 1H), 7.69 (br d, J=4.0 Hz, 1H), 7.16 (d, J=4.0 Hz, 1H), 6.75-6.64 (m, 2H), 4.25 (br s, 2H), 4.15-4.05 (m, 1H), 4.03 (br s, 2H), 3.62-3.44 (m, 1H), 3.22-3.06 (m, 1H), 3.04-2.91 (m, 1H), 2.79-2.59 (m, 2H), 1.96-1.70 (m, 4H), 1.54-1.43 (m, 1H).

### Example 913

### Preparation of (R)-3-(4-amino-5-(3-(5-chlorothiophene-2-carboxamido)piperidin-1-yl)pyrrolo[2,1-f][1,2,4]triazin-7-yl)-2,5-dihydro-1H-pyrrole-1-carboxamide

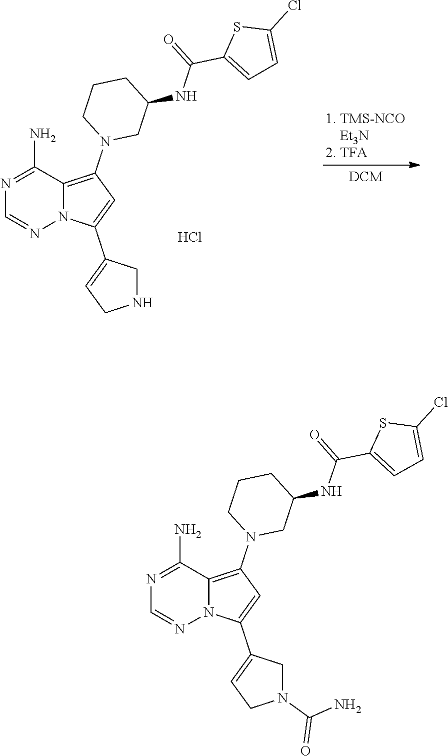 [SMILES: Nc1ncnn2c(C3=CCNC3)cc(N3CCC[C@@H](NC(=O)c4ccc(Cl)s4)C3)c12 ; Cl ; NC(=O)N1CC=C(c2cc(N3CCC[C@@H](NC(=O)c4ccc(Cl)s4)C3)c3c(N)ncnn23)C1]

A solution of (R)—N-(1-(4-amino-7-(2,5-dihydro-1H-pyrrol-3-yl)pyrrolo[2,1-f][1,2,4]triazin-5-yl)piperidin-3-yl)-5-chlorothiophene-2-carboxamide hydrochloride (20 mg, 0.042 mmol) and triethylamine (0.029 mL, 0.208 mmol) in dichloromethane (1 mL) was treated with isocyanatotrimethylsilane (7.3 μl, 0.046 mmol). The reaction was stirred at rt for 2 h. The mixture was treated with TFA (1 mL), and the reaction was stirred at rt for 1 h. The mixture was concentrated. The crude material was purified via preparative HPLC to yield the title compound (3.1 mg, 15% yield). LC-MS Method A: RT=1.51 min, [M+H]⁺=487.1; LC-MS Method B: RT=1.33 min, [M+H]⁺=486.9; ¹H NMR (500 MHz, DMSO-d₆) δ 8.45 (br d, J=7.3 Hz, 1H), 8.04-7.90 (m, 1H), 7.86 (s, 1H), 7.72 (d, J=4.0 Hz, 1H), 7.18 (d, J=4.0 Hz, 1H), 6.95-6.81 (m, 1H), 6.75 (br s, 1H), 6.60 (br s, 1H), 5.84 (s, 2H), 4.46 (br s, 2H), 4.24 (br s, 2H), 4.17-4.05 (m, 1H), 3.39-3.27 (m, 1H), 3.17 (br dd, J=11.7, 3.8 Hz, 1H), 3.07-2.98 (m, 1H), 2.74-2.58 (m, 2H), 1.96-1.74 (m, 3H), 1.56-1.44 (m, 1H).

### Example 914

### Preparation of (R)—N-(1-(7-(1-(acetylglycyl)-2,5-dihydro-1H-pyrrol-3-yl)-4-aminopyrrolo[2,1-f][1,2,4]triazin-5-yl)piperidin-3-yl)-5-chlorothiophene-2-carboxamide

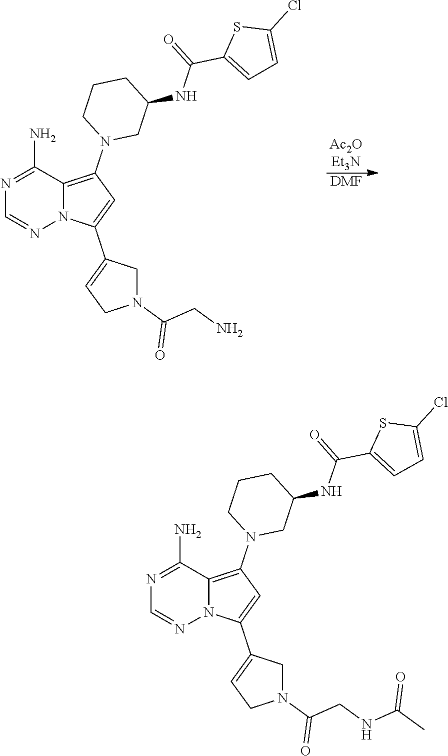

A solution of Example 881 (0.016 g, 0.031 mmol) and triethylamine (0.022 mL, 0.155 mmol) in DMF (2 mL) was treated with acetic anhydride (3.51 μl, 0.037 mmol). The reaction was stirred at rt for 2 h. The mixture was filtered, and the filtrate was purified via preparative HPLC to yield the title compound. LC-MS Method A: RT=1.54 min, [M+H]⁺=543.1; LC-MS Method B: RT=1.32 min, [M+H]⁺=543.1; ¹H NMR (500 MHz, DMSO-d₆) δ 8.46 (br dd, J=7.3, 3.4 Hz, 1H), 8.09-8.01 (m, 1H), 8.00-7.90 (m, 1H), 7.88 (d, J=3.4 Hz, 1H), 7.71 (br d, J=3.7 Hz, 1H), 7.18 (d, J=4.0 Hz, 1H), 7.01-6.83 (m, 1H), 6.79 (br d, J=15.6 Hz, 1H), 6.75 (d, J=10.1 Hz, 1H), 4.71 (br s, 1H), 4.54 (br s, 1H), 4.49 (br s, 1H), 4.32 (br s, 1H), 4.18-4.06 (m, 1H), 4.00-3.89 (m, 2H), 3.25-3.13 (m, 1H), 3.08-2.97 (m, 1H), 2.76-2.57 (m, 2H), 2.01-1.73 (m, 6H), 1.56-1.43 (m, 1H).

### Example 915

### Preparation of (R)—N-(1-(7-(1-(3-acetamidopropanoyl)-2,5-dihydro-1H-pyrrol-3-yl)-4-aminopyrrolo[2,1-f][1,2,4]triazin-5-yl)piperidin-3-yl)-5-chlorothiophene-2-carboxamide

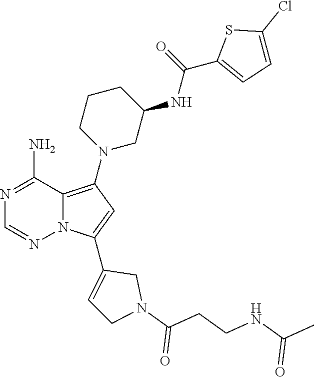 [SMILES: CC(=O)NCCC(=O)N1CC=C(c2cc(N3CCC[C@@H](NC(=O)c4ccc(Cl)s4)C3)c3c(N)ncnn23)C1]

The title compound was prepared from Example 883 using the procedure described for the preparation of Example 914. LC-MS Method A: RT=1.55 min, [M+H]⁺=557.1; LC-MS Method B: RT=1.27 min, [M+H]⁺=557.2; ¹H NMR (500 MHz, DMSO-d₆) δ 8.48 (br d, J=7.6 Hz, 1H), 7.96-7.88 (m, 1H), 7.86 (s, 1H), 7.69 (br d, J=3.4 Hz, 1H), 7.19-7.14 (m, 1H), 6.99-6.82 (m, 1H), 6.77 (br d, J=8.9 Hz, 1H), 6.71 (d, J=5.8 Hz, 1H), 4.66 (br s, 1H), 4.51 (br s, 1H), 4.44 (br s, 1H), 4.29 (br s, 1H), 4.14-4.04 (m, 1H), 3.55-3.47 (m, 1H), 3.35-3.27 (m, 1H), 3.22-3.13 (m, 2H), 3.07-2.95 (m, 1H), 2.75-2.59 (m, 2H), 2.47 (br t, J=6.9 Hz, 1H), 1.97-1.75 (m, 6H), 1.57-1.44 (m, 1H).

### Example 916

### Preparation of (cis)-N-[(3R)-1-[7-(1-acetyl-2,5-dihydro-1H-pyrrol-3-yl)-4-aminopyrrolo[2,1-f][1,2,4]triazin-5-yl]piperidin-3-yl]bicyclo[3.1.0]hexane-1-carboxamide

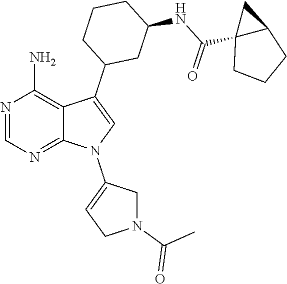 [SMILES: CC(=O)N1CC=C(n2cc(C3CCC[C@@H](NC(=O)[C@@]45CCC[C@@H]4C5)C3)c3c(N)ncnc32)C1]

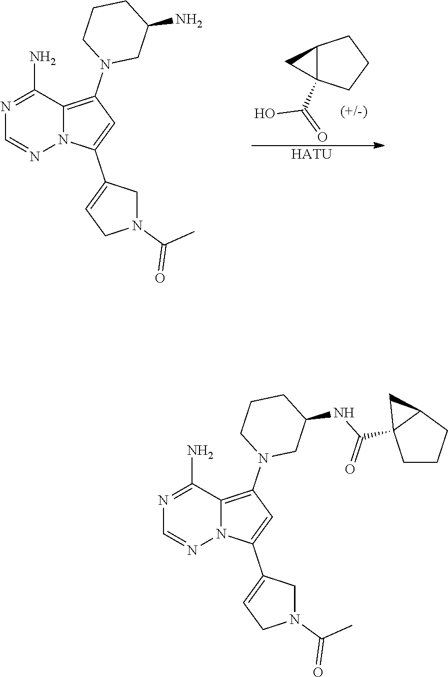

To a solution of Intermediate 32, HCl salt (18 mg, 0.032 mmol) and rac-(1R,5R)-bicyclo[3.1.0]hexane-1-carboxylic acid (4.2 mg, 0.033 mmol) in DMF (0.5 mL), were added DIEA (0.028 mL, 0.16 mmol) and HATU (13.2 mg, 0.035 mmol). The mixture was stirred at rt for 2.5 h. The mixture was quenched with MeOH (0.5 mL), then was acidified with a few drops of TFA. The mixture was filtered, then was purified by preparative HPLC to afford Example 916 (6.8 mg, 48% yield). LC-MS Method A: RT=1.45 min, [M+H]⁺=450.30; LC-MS Method B: RT=1.22 min, [M+H]⁺=450.20; ¹H NMR (500 MHz, DMSO-d₆) δ 7.86 (d, J=1.2 Hz, 1H), 7.19 (br d, J=7.0 Hz, 1H), 6.76 (br d, J=12.5 Hz, 1H), 6.67 (d, J=16.5 Hz, 1H), 4.73-4.21 (m, 4H), 3.98-3.90 (m, 1H), 3.10-2.93 (m, 1H), 2.08-1.98 (m, 3H), 1.96-1.86 (m, 1H), 1.84-1.56 (m, 8H), 1.51-1.38 (m, 1H), 1.27-1.15 (m, 1H), 1.02-0.98 (m, 1H), 0.66 (br s, 1H).

### Example 917

### Preparation of N-[(3R)-1-[7-(1-acetyl-2,5-dihydro-1H-pyrrol-3-yl)-4-aminopyrrolo[2,1-f][1,2,4]triazin-5-yl]piperidin-3-yl]-5-chloro-3-[(hexahydro-1H-pyrrolizin-7a-yl)methoxy]thiophene-2-carboxamide

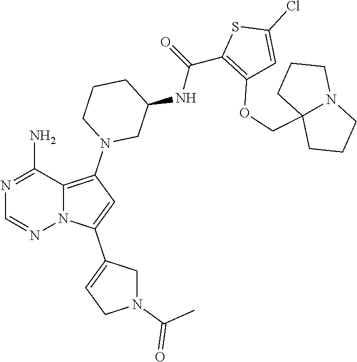 [SMILES: CC(=O)N1CC=C(c2cc(N3CCC[C@@H](NC(=O)c4sc(Cl)cc4OCC45CCCN4CCC5)C3)c3c(N)ncnn23)C1]

### Example 917A. methyl 5-chloro-3-((tetrahydro-1H-pyrrolizin-7a(5H)-yl)methoxy)thiophene-2-carboxylate

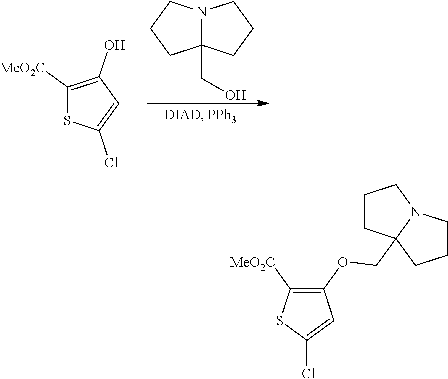

To a solution of methyl 5-chloro-3-hydroxythiophene-2-carboxylate (300 mg, 1.56 mmol), (tetrahydro-1H-pyrrolizin-7a(5H)-yl)methanol (0.238 mL, 1.71 mmol), and triphenylphosphine (613 mg, 2.34 mmol) in THF (4 mL) at 0° C., was added DIAD (0.459 mL, 2.34 mmol), dropwise. The mixture was stirred at 0° C. for 15 min, then was removed from the ice bath and stirred at rt for 2.5 h. MeOH (0.1 mL) was added, then the mixture was concentrated. The crude product was purified by flash chromatography (1 to 20% MeOH/DCM gradient) to afford Example 917A (420 mg, 85% yield) as a colorless oil. MS: [M+H]⁺=315.9.

### Example 917B. 5-chloro-3-((tetrahydro-1H-pyrrolizin-7a(5H)-yl)methoxy)thiophene-2-carboxylic acid, TFA salt

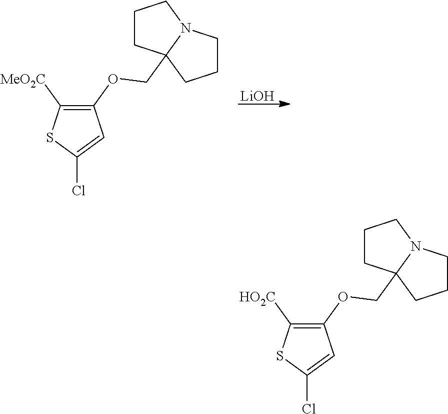

To a solution Example 917A (415 mg, 1.31 mmol) in THF (4 mL), were added LiOH (63 mg, 2.63 mmol) and Water (1 mL). MeOH (0.5 mL) was added. The mixture was stirred at rt for 20 h. 1N aq. HCl (2.63 mL, 2.63 mmol) was added, then the volatiles were evaporated. The crude product was purified by preparative HPLC to afford Example 917B (318 mg, 58% yield) as colorless needles. MS: [M+H]⁺=301.9; ¹H NMR (500 MHz, METHANOL-d₄) δ 7.08 (s, 1H), 4.30 (s, 2H), 3.73 (dt, J=11.5, 6.8 Hz, 2H), 3.28 (dt, J=11.4, 6.3 Hz, 2H), 2.35-2.26 (m, 2H), 2.25-2.16 (m, 4H), 2.11-2.04 (m, 2H).

### Example 917

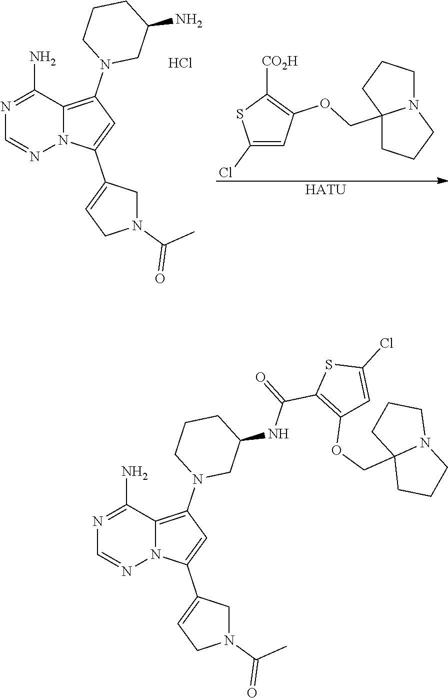

To a solution of Intermediate 32, HCl salt (20 mg, 0.053 mmol), Example 917B (24.2 mg, 0.058 mmol), and IEA (0.046 mL, 0.265 mmol) in DMF (1 mL), was added HATU (22.1 mg, 0.058 mmol). The mixture was stirred at rt for 2 h. The mixture was quenched with MeOH (0.5 mL), then filtered. The filtrate was purified by preparative HPLC to afford Example 917 (15.1 mg, 45% yield). LC-MS Method A: RT=1.57 min, [M+H]⁺=625.10; LC-MS Method B: RT=1.12 min, [M+H]⁺=625.20; ¹H NMR (500 MHz, DMSO-d₆) δ 7.85 (s, 1H), 7.41 (br s, 1H), 7.32 (s, 1H), 6.76 (br d, J=13.1 Hz, 1H), 6.68 (br d, J=13.4 Hz, 1H), 4.69-4.23 (m, 5H), 4.11 (br s, 1H), 3.95-3.86 (m, 2H), 2.93-2.84 (m, 2H), 2.04 (s, 15H), 2.00 (s, 1.5H), 1.92-1.73 (m, 6H), 1.72-1.62 (m, 2H), 1.56 (br d, J=3.1 Hz, 2H).

### Example 918

### Preparation of 4-{[(3aR,5R,7aS)-1-methyl-octahydro-1H-indol-5-yl]oxy}-N-[(3R)-1-[4-amino-7-(4-carbamoyl-3-fluorophenyl)pyrrolo[2,1-f][1,2,4]triazin-5-yl]piperidin-3-yl]-2-methyl-1,3-thiazole-5-carboxamide

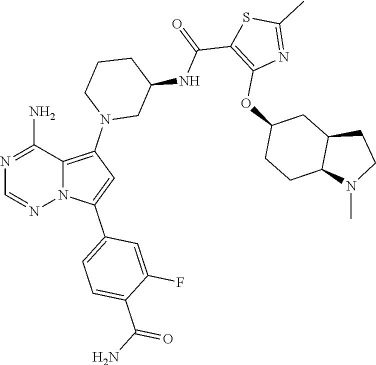 [SMILES: Cc1nc(O[C@@H]2CC[C@H]3[C@H](CCN3C)C2)c(C(=O)N[C@@H]2CCCN(c3cc(-c4ccc(C(N)=O)c(F)c4)n4ncnc(N)c34)C2)s1]

### Example 918A. rac-ethyl 4-(((3aR,5R,7aS)-1-(tert-butoxycarbonyl)octahydro-1H-indol-5-yl)oxy)-2-methylthiazole-5-carboxylate

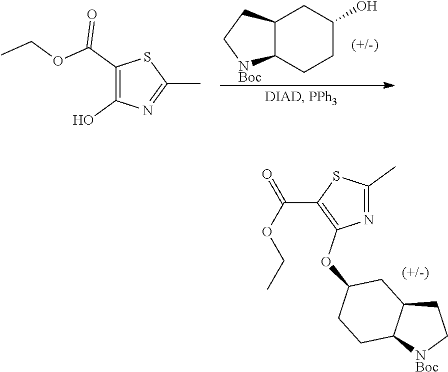

To a solution of rac-tert-butyl (3aS,5R,7aR)-5-hydroxyoctahydro-1H-indole-1-carboxylate (0.037 g, 0.153 mmol) and ethyl 4-hydroxy-2-methylthiazole-5-carboxylate (0.029 g, 0.153 mmol) in anhydrous THF (0.77 mL) at 0° C. was added triphenylphosphine (0.044 g, 0.169 mmol), followed by dropwise addition of DIAD (0.033 mL, 0.169 mmol). The reaction was stirred for 15 min, then was allowed to come to it and stirred for 3 h. The mixture was concentrated in vacuo, and the residue was purified by flash chromatography (0% to 45% EtOAc/hexanes gradient) to yield Example 918A (43 mg, 68% yield) as a clear colorless glass. MS: [M+H]⁺=411.1.

### Example 918B. rac-4-(((3aR,5R,7aS)-1-(tert-butoxycarbonyl)octahydro-1H-indol-5-yl)oxy)-2-methylthiazole-5-carboxylic acid

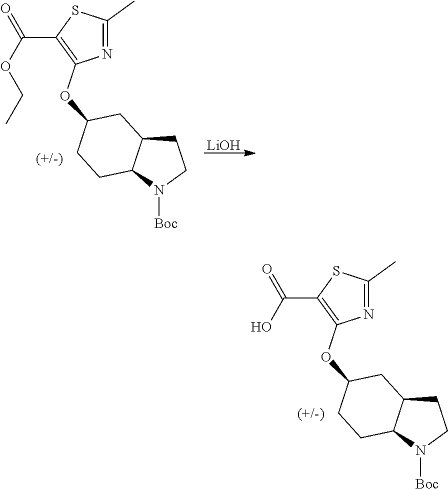

To a solution of Example 918A (43 mg, 0.105 mmol) in a mixture of THF (0.3 mL) and Methanol (0.1 mL) was added 2M aq. lithium hydroxide (0.16 mL, 0.32 mmol). The mixture was stirred at 60° C. for 1 h, then was evaporated to remove organic solvents. The residue was partitioned between water and Et₂O. The organic phase was extracted with water. The combined aqueous phase was acidified (pH 3) with saturated NH₄Cl/1N HCl, then was extracted with EtOAc (3×). The combined organic phase was dried (Na₂SO₄), filtered, and concentrated to afford Example 918B (39 mg, 97% yield) as a white solid. MS: [M+H]⁺=383.0.

### Example 918C. tert-butyl (3aR,5R,7aS)-5-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)octahydro-1H-indole-1-carboxylate

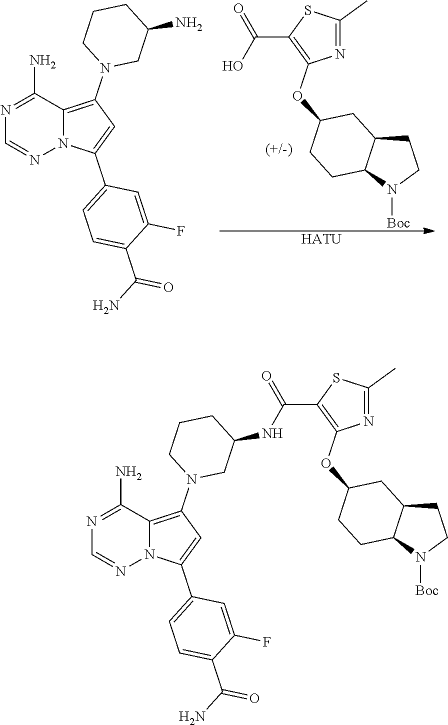 [SMILES: NC(=O)c1ccc(-c2cc(N3CCC[C@@H](N)C3)c3c(N)ncnn23)cc1F ; Cc1nc(O[C@@H]2CC[C@H]3[C@H](CCN3C(=O)OC(C)(C)C)C2)c(C(=O)O)s1 ; "HATU" ; Cc1nc(O[C@@H]2CC[C@H]3[C@H](CCN3C(=O)OC(C)(C)C)C2)c(C(=O)N[C@@H]2CCCN(c3cc(-c4ccc(C(N)=O)c(F)c4)n4ncnc(N)c34)C2)s1]

To a solution of Example 139D, 2 HCl (47.4 mg, 0.107 mmol) and Example 918B (39 mg, 0.102 mmol) in DMF (1.0 mL) at 0° C., was added DIEA (80 μL, 0.459 mmol), followed by HATU (46.5 mg, 0.122 mmol). The mixture stirred 5 min. The mixture was diluted with MeOH and purified by preparative HPLC to afford Example 918C (60 mg, 80% yield) as a white solid. MS: [M+H]⁺=734.5.

### Example 918D. N—((R)-1-(4-amino-7-(4-carbamoyl-3-fluorophenyl)pyrrolo[2,1-f][1,2,4]triazin-5-yl)piperidin-3-yl)-2-methyl-4-(((3aR,5R,7aS)-octahydro-1H-indol-5-yl)oxy)thiazole-5-carboxamide 2HCl

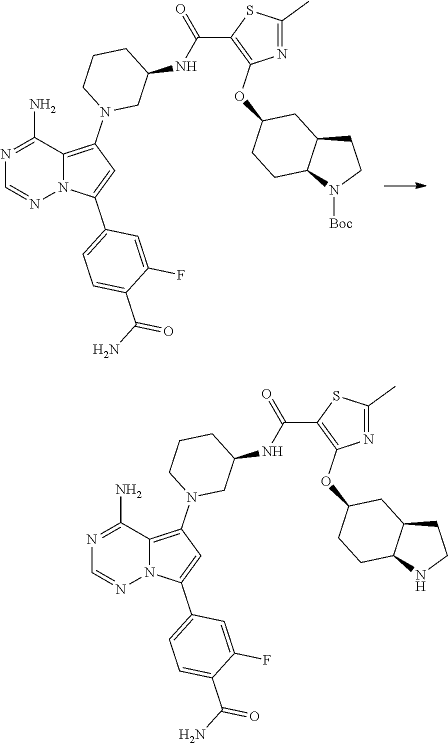 [SMILES: Cc1nc(O[C@@H]2CC[C@H]3[C@H](CCN3C(=O)OC(C)(C)C)C2)c(C(=O)N[C@@H]2CCCN(c3cc(-c4ccc(C(N)=O)c(F)c4)n4ncnc(N)c34)C2)s1 ; Cc1nc(O[C@@H]2CC[C@@H]3NCC[C@@H]3C2)c(C(=O)N[C@@H]2CCCN(c3cc(-c4ccc(C(N)=O)c(F)c4)n4ncnc(N)c34)C2)s1]

To a solution of Example 918C (60 mg, 0.082 mmol) in DCM (0.8 mL) was added 4N HCl in dioxane (0.41 mL, 1.6 mmol). The mixture was stirred for 2 h, then was evaporated. The residue was co-evaporated with DCM/MeOH to afford Example 918B (51 mg, 88% yield), which was used without further purification. MS: [M+H]⁺=634.0.

### Example 918

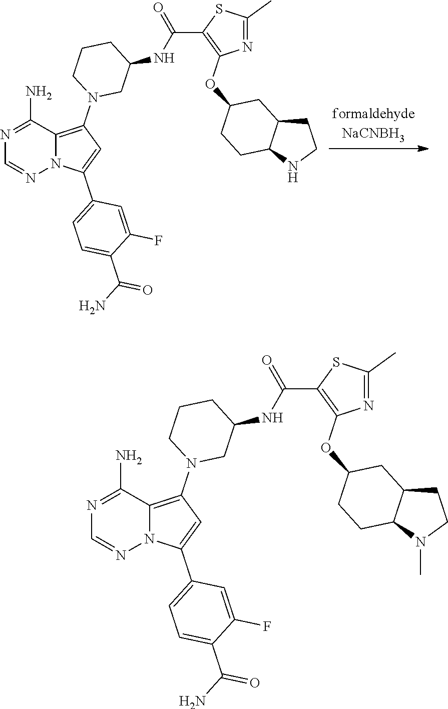 [SMILES: Cc1nc(O[C@@H]2CC[C@@H]3NCC[C@@H]3C2)c(C(=O)N[C@@H]2CCCN(c3cc(-c4ccc(C(N)=O)c(F)c4)n4ncnc(N)c34)C2)s1 ; Cc1nc(O[C@@H]2CC[C@H]3[C@H](CCN3C)C2)c(C(=O)N[C@@H]2CCCN(c3cc(-c4ccc(C(N)=O)c(F)c4)n4ncnc(N)c34)C2)s1]

To a stirred solution of Example 918D (31 mg, 0.044 mmol), formaldehyde (37 wt. % solution in H₂O, 13 μL, 0.175 mmol) and acetic acid (10 μl, 0.175 mmol) in MeOH (0.6 mL) at 0° C., was added sodium cyanoborohydride (6.9 mg, 0.11 mmol). The reaction mixture was stirred at rt for 4 h. The reaction mixture was evaporated, then partitioned between DCM and 1.5M K₂HPO₄. The aqueous phase was extracted with DCM (3×). The combined organic phase was dried (Na₂SO₄), filtered and concentrated. The residue was purified by preparative HPLC to afford Example 918 (7.3 mg, 26% yield). LC-MS Method A: RT=1.54 min, [M+H]⁺+=648.2; LC-MS Method B: RT=1.15 min, [M+H]⁺=648.2; 1H NMR (500 MHz, DMSO-d₆) 8.11 (d, J=12.8 Hz, 1H), 8.00 (dd, J=8.3, 1.4 Hz, 1H), 7.93 (s, 1H), 7.73 (t, J=8.2 Hz, 1H), 7.66 (br s, 1H), 7.62 (br s, 1H), 7.24 (s, 1H), 4.91-4.82 (m, 1H), 4.32-4.22 (m, 1H), 3.40-3.34 (m, 1H), 3.25-3.16 (m, 1H), 2.98-2.93 (m, 1H), 2.60 (s, 3H), 2.16 (s, 3H), 2.15-2.02 (m, 3H), 2.00-1.74 (m, 7H), 1.67-1.51 (m, 3H), 1.45-1.35 (m, 1H), 1.24-1.11 (m, 1H).

### Example 919

### Preparation of N-[(3R)-1-{4-amino-7-[5-fluoro-6-(1H-1,2,3,4-tetrazol-1-yl)pyridin-3-yl]pyrrolo[2,1-f][1,2,4]triazin-5-yl}piperidin-3-yl]-4-[3-(dimethylamino)propoxy]-2-methyl-1,3-thiazole-5-carboxamide

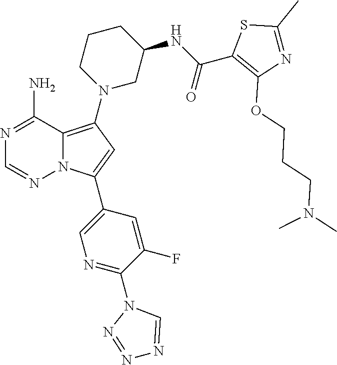 [SMILES: Cc1nc(OCCCN(C)C)c(C(=O)N[C@@H]2CCCN(c3cc(-c4cnc(-n5cnnn5)c(F)c4)n4ncnc(N)c34)C2)s1]

### Example 919A. ethyl 4-(3-(dimethylamino)propoxy)-2-methylthiazole-5-carboxylate

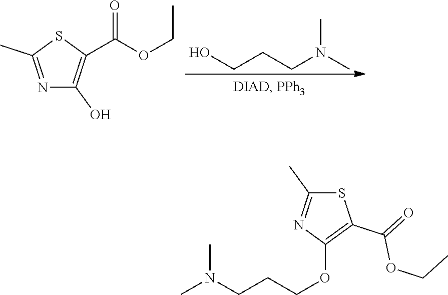

To a solution of ethyl 4-hydroxy-2-methylthiazole-5-carboxylate (556 mg, 2.97 mmol) in THF (10 mL), was added 3-(dimethylamino)propan-1-ol (306 mg, 2.97 mmol), followed by addition of triphenylphosphine (1168 mg, 4.45 mmol) and DIAD (0.866 mL, 4.45 mmol). The mixture was stirred at rt for 6 days. The reaction mixture was diluted with EtOAc, washed with H₂O (2×) and brine. The organic phase was dried over Na₂SO₄, filtered and concentrated. The crude product was purified by flash chromatography (0% to 20% MeOH/DCM gradient) to afford Example 919A (640 mg, 79% yield). MS: [M+H]⁺=273.2; ¹H NMR (500 MHz, CDCl₃) δ 4.48 (t, J=6.5 Hz, 2H), 4.27 (q, J=7.1 Hz, 2H), 2.60 (s, 3H), 2.52-2.40 (m, 2H), 2.25 (s, 6H), 2.04-1.87 (m, 2H), 1.33 (t, J=7.1 Hz, 3H).

### Example 919B. Sodium 4-(3-(dimethylamino)propoxy)-2-methylthiazole-5-carboxylate

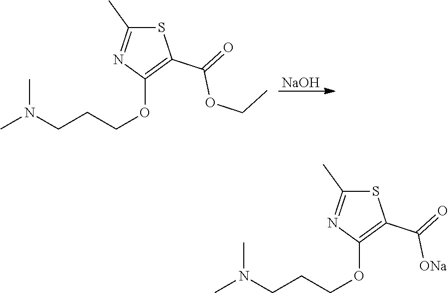

To a solution of Example 919A (640 mg, 2.35 mmol) in THF/MeOH (3:1) (12 mL) was added NaOH (1N) (3.5 mL, 3.5 mmol). The mixture was stirred at room temperature overnight. The mixture was concentrated to give Example 919B (743 mg) which was used directly in the next step. MS: [M+H]⁺=245.1

### Example 919C. (R)—N-(1-(4-amino-7-bromopyrrolo[2,1-f][1,2,4]triazin-5-yl)piperidin-3-yl)-4-(3-(dimethylamino)propoxy)-2-methylthiazole-5-carboxamide, 2TFA

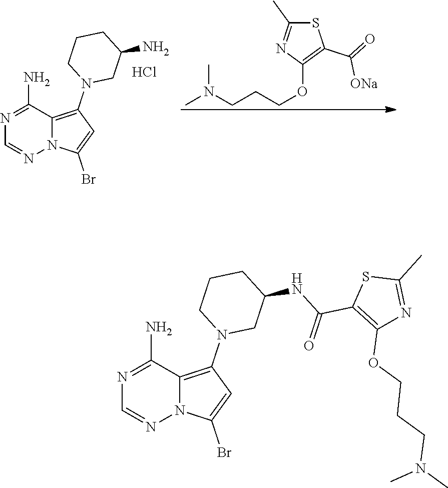

To a solution of Example 919B (460 mg, 1.73 mmol) in DMF (11.5 mL) was added Intermediate 61. HCl (400 mg, 1.15 mmol), DIEA (1.01 mL, 5.75 mmol) and HATU (656 mg, 1.73 mmol), sequentially. The mixture was stirred at rt 24 h. The mixture was diluted with EtOAc, washed with water and brine. The organic layer was separated, dried (Na₂SO₄), filtered and concentrated. The reside was purified by preparative HPLC to give Example 919C (388 mg, 53% yield) as a yellow solid. MS: [M+H]⁺=536.9

### Example 919

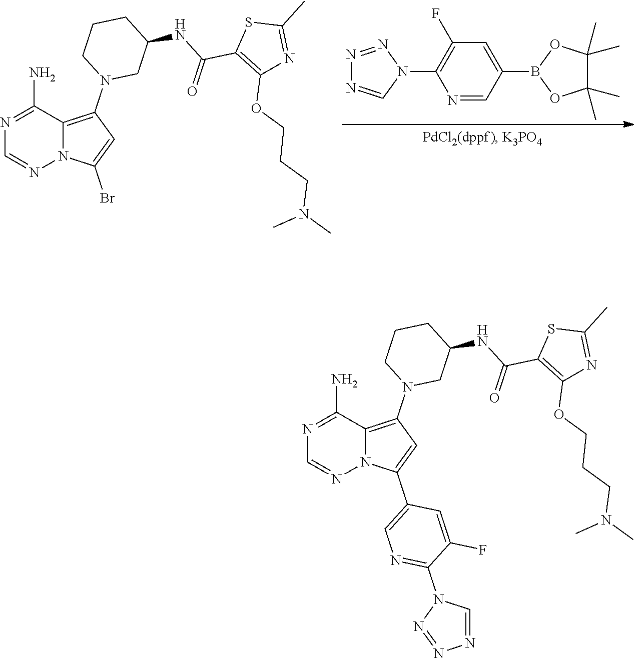 [SMILES: Cc1nc(OCCCN(C)C)c(C(=O)N[C@@H]2CCCN(c3cc(Br)n4ncnc(N)c34)C2)s1 ; CC1(C)OB(c2cnc(-n3cnnn3)c(F)c2)OC1(C)C ; Cc1nc(OCCCN(C)C)c(C(=O)N[C@@H]2CCCN(c3cc(-c4cnc(-n5cnnn5)c(F)c4)n4ncnc(N)c34)C2)s1]

To a solution of 3-fluoro-5-(4,4,5,5-tetramethyl-1,3,2-dioxaborolan-2-yl)-2-(1H-tetrazol-1-yl)pyridine (21.7 mg, 0.074 mmol) in THF (0.3 mL) and H₂O (0.6 mL) was added Example 919C (20 mg, 0.037 mmol) followed by addition of K₃PO₄ (15.8 mg, 0.074 mmol) and PdCl₂(dppf) (5.5 mg, 7.4 μmol) at it. The reaction was stirred under N2 at 70° C. for 2 h. The mixture was diluted with DMF, filtered and purified by preparative HPLC to afford Example 919 (8.9 mg, 36% yield). LC-MS Method A: RT=1.47 min, [M+H]⁺=622.2; LC-MS Method B: RT=1.13 min, [M+H]⁺=622.3; ¹H NMR (500 MHz, DMSO-d₆) δ 10.09 (s, 1H), 9.22 (s, 1H), 8.91 (dd, J=12.0, 1.6 Hz, 1H), 7.99 (s, 1H), 7.45 (s, 1H), 7.30 (br s, 1H), 4.50 (br t, J=6.1 Hz, 2H), 4.16 (br s, 1H), 3.49-3.36 (m, 1H), 3.22-3.12 (m, 2H), 3.03 (br s, 1H), 2.75 (br s, 6H), 2.60 (s, 3H), 2.19-2.08 (m, 2H), 1.97-1.88 (m, 1H), 1.84 (br s, 2H), 1.60 (br s, 1H).

### Example 920

### N—((R)-1-(7-(1-acetyl-2,5-dihydro-1H-pyrrol-3-yl)-4-aminopyrrolo[2,1-f][1,2,4]triazin-5-yl)piperidin-3-yl)-5-cyano-3-(((1R,3s,5S)-8-methyl-8-azabicyclo[3.2.1]octan-3-yl)oxy)thiophene-2-carboxamide

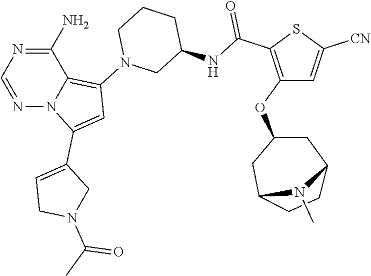 [SMILES: CC(=O)N1CC=C(c2cc(N3CCC[C@@H](NC(=O)c4sc(C#N)cc4O[C@@H]4C[C@H]5CC[C@@H](C4)N5C)C3)c3c(N)ncnn23)C1]

Coupling of Intermediate 32, TFA salt with Intermediate 65 using HATU afforded Example 920. LC-MS Method B: ESI m/z 616 (M+H)⁺. RT=1.31 min; ¹H NMR (500 MHz, DMSO-d₆) δ 8.28 (s, 1H), 7.96 (s, 1H), 7.94 (s, 1H), 7.47-7.35 (m, 1H), 6.83-6.73 (m, 2H), 5.01-4.85 (m, 1H), 4.69 (br s, 1H), 4.49 (br d, J=5.8 Hz, 2H), 4.28 (br s, 1H), 4.24-4.15 (m, 1H), 3.99 (br s, 2H), 3.17-3.12 (m, 1H), 3.07-3.00 (m, 1H), 2.85-2.76 (m, 2H), 2.70 (br s, 3H), 2.44-2.37 (m, 2H), 2.26-2.08 (m, 6H), 2.04 (d, J=19.3 Hz, 3H), 1.98-1.85 (m, 2H), 1.83-1.74 (m, 1H), 1.60-1.47 (m, 1H).

### Example 921

### (R)—N-(1-(7-(1-acetyl-2,5-dihydro-1H-pyrrol-3-yl)-4-aminopyrrolo[2,1-f][1,2,4]triazin-5-yl)piperidin-3-yl)-5-chloro-3-(2-hydroxy-2-methylpropoxythiophene-2-carboxamide TFA salt

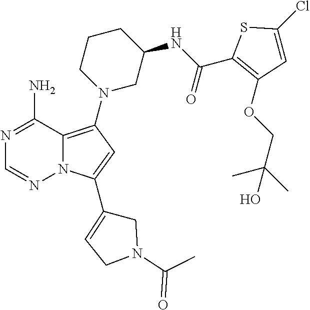 [SMILES: CC(=O)N1CC=C(c2cc(N3CCC[C@@H](NC(=O)c4sc(Cl)cc4OCC(C)(C)O)C3)c3c(N)ncnn23)C1]

Example 921 was synthesized by following the procedure analogous as described in previous examples. LC-MS Method B: ESI m/z 574 (M+H)⁺, RT=1.39 min; ¹H NMR (500 MHz, DMSO-d₆) δ 7.96 (s, 1H), 7.66 (br d, J=7.7 Hz, 1H), 7.31 (s, 1H), 6.83-6.71 (m, 2H), 4.69 (br d, J=1.2 Hz, 1H), 4.49 (br d, J=11.6 Hz, 2H), 4.28 (br s, 1H), 4.21-4.10 (m, 1H), 3.99 (s, 2H), 3.25-3.18 (m, 1H), 3.05-2.98 (m, 1H), 2.77-2.60 (m, 2H), 2.03 (d, J=19.4 Hz, 3H), 1.97-1.90 (m, 1H), 1.85 (br d, J=2.0 Hz, 2H), 1.52-1.38 (m, 1H), 1.20 (s, 6H).

### Example 922

### N—((R)-1-(7-(4-(1H-1,2,3-triazol-1-yl)phenyl)-4-aminopyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide, TFA salt

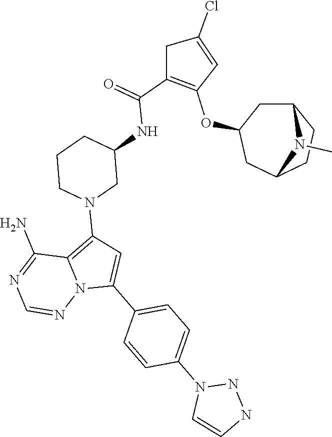 [SMILES: CN1[C@@H]2CC[C@H]1C[C@H](OC1=C(C(=O)N[C@@H]3CCCN(c4cc(-c5ccc(N6C=CNN6)cc5)n5ncnc(N)c45)C3)CC(Cl)=C1)C2]

Example 922 was synthesized by following the procedure analogous as described in previous examples. LC-MS Method B: ESI m/z 659 (M+H)⁺, RT=1.22 min; ¹H NMR (500 MHz, DMSO-d₆) δ 10.00 (br d, J=3.7 Hz, 1H), 8.84 (s, 1H), 8.28 (d, J=8.9 Hz, 2H), 8.04-7.98 (m, 3H), 7.95 (s, 1H), 7.53 (s, 1H), 7.19 (br s, 1H), 4.93-4.78 (m, 1H), 4.24-4.11 (m, 1H), 3.95 (br s, 2H), 3.27-3.21 (m, 1H), 3.08 (br d, J=10.7 Hz, 1H), 2.95-2.74 (m, 2H), 2.69 (br s, 3H), 2.43-2.34 (m, 2H), 2.21 (br d, J=10.4 Hz, 2H), 2.13 (br d, J=18.3 Hz, 2H), 2.08-2.01 (m, 2H), 1.99-1.86 (m, 2H), 1.78 (s, 1H), 1.58-1.46 (m, 1H).

### Example 923

### (R)—N-(1-(7-(1-acetyl-2,5-dihydro-1H-pyrrol-3-yl)-4-aminopyrrolo[2,1-f][1,2,4]triazin-5-yl)piperidin-3-yl)-4H-thieno[3,2-b]pyrrole-2-carboxamide

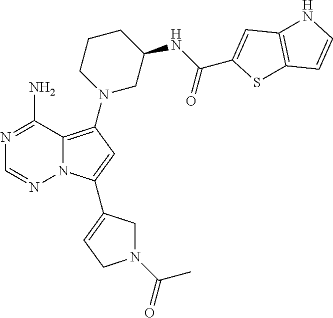 [SMILES: CC(=O)N1CC=C(c2cc(N3CCC[C@@H](NC(=O)c4cc5[nH]ccc5s4)C3)c3c(N)ncnn23)C1]

Example 923 was synthesized by following the procedure analogous as described in previous examples. LC-MS Method B: ESI m/z 491 (M+H)⁺, RT=1.16 min; ¹H NMR (500 MHz, DMSO-d₆) δ 11.38 (br s, 1H), 8.23 (br d, J=7.6 Hz, 1H), 7.86 (s, 1H), 7.80 (s, 1H), 7.23 (br s, 1H), 6.81-6.75 (m, 1H), 6.70 (d, J=9.8 Hz, 1H), 6.39 (s, 1H), 4.69 (br s, 1H), 4.48 (br d, J=12.8 Hz, 2H), 4.27 (br s, 1H), 4.16-4.05 (m, 1H), 3.23-3.18 (m, 1H), 3.03-2.95 (m, 1H), 2.73-2.60 (m, 2H), 2.05 & 2.01 (s, 3H), 1.96-1.73 (m, 3H), 1.58-1.44 (m, 1H).

### Example 924

### N—((R)-1-(4-amino-7-(1-oxo-1,3-dihydroisobenzofuran-5-yl)pyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide

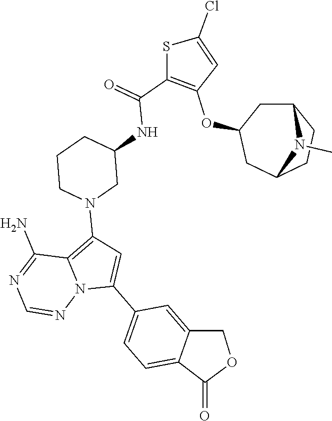 [SMILES: CN1[C@@H]2CC[C@H]1C[C@H](Oc1cc(Cl)sc1C(=O)N[C@@H]1CCCN(c3cc(-c4ccc5c(c4)COC5=O)n4ncnc(N)c34)C1)C2]

Example 924 was synthesized by following the procedure analogous as described in previous examples. LC-MS Method B: ESI m/z 648 (M+H)⁺, RT=1.28 min; ¹H NMR (500 MHz, DMSO-d₆) δ 8.40 (s, 1H), 8.26 (br d, J=8.5 Hz, 1H), 7.96-7.84 (m, 2H), 7.39 (s, 1H), 7.22 (s, 1H), 5.44 (s, 2H), 4.71-4.65 (m, 1H), 4.21-4.16 (m, 1H), 3.86 (br s, 3H), 3.16 (br s, 2H), 2.95-2.86 (m, 2H), 2.15 (br s, 3H), 2.03-1.96 (m, 2H), 1.95-1.88 (m, 2H), 1.82 (br d, J=5.5 Hz, 2H), 1.74-1.52 (m, 6H).

### Example 925

### N-((3R,5R)-1-(4-amino-7-(3-fluoro-4-(1H-tetrazol-1-yl)phenyl)pyrrolo[2,1-f][1,2,4]triazin-5-yl)-5-fluoropiperidin-3-yl)-5-chloro-3-(((1R,3s,5S)-8-methyl-8-azabicyclo[3.2.1]octan-3-yl)oxy)thiophene-2-carboxamide, TFA salt

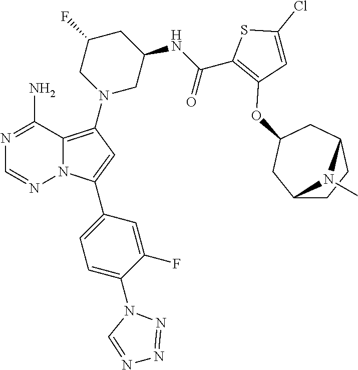 [SMILES: CN1[C@@H]2CC[C@H]1C[C@H](Oc1cc(Cl)sc1C(=O)N[C@@H]1C[C@@H](F)CN(c3cc(-c4ccc(-n5cnnn5)c(F)c4)n4ncnc(N)c34)C1)C2]

Example 925 was synthesized by following the procedure analogous as described in previous examples. LC-MS Method B: ESI m/z 696 (M+H)⁺, RT=1.32 min; ¹H NMR (500 MHz, DMSO-d₆) δ 9.98 (d, J=1.5 Hz, 1H), 9.86 (br s, 1H), 8.42 (dd, J=12.6, 1.8 Hz, 1H), 8.24 (dd, J=8.5, 1.5 Hz, 1H), 8.03-7.95 (m, 2H), 7.57 (s, 1H), 7.36 (s, 1H), 7.18 (br d, J=7.6 Hz, 1H), 5.25-5.07 (m, 1H), 4.95-4.83 (m, 1H), 4.41-4.27 (m, 1H), 3.48-3.32 (m, 2H), 3.01-2.83 (m, 2H), 2.71 (d, J=5.0 Hz, 3H), 2.42-2.32 (m, 3H), 2.26-2.17 (m, 3H), 2.15-2.01 (m, 4H).

### Example 926

### N—((R)-1-(4-amino-7-(2,5-dihydrofuran-3-yl)pyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide, TFA salt

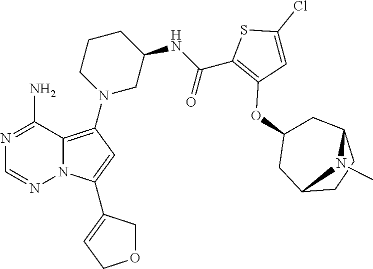 [SMILES: CN1[C@@H]2CC[C@H]1C[C@H](Oc1cc(Cl)sc1C(=O)N[C@@H]1CCCN(c3cc(C4=CCOC4)n4ncnc(N)c34)C1)C2]

Example 926 was synthesized by following the procedure analogous as described in previous examples. LC-MS Method B: ESI m/z 584 (M+H)⁺, RT=1.17 min; ¹H NMR (500 MHz, DMSO-d₆) δ 10.14 (br s, 1H), 9.06 (br s, 1H), 8.03 (s, 1H), 7.58 (s, 1H), 7.13 (br d, J=6.1 Hz, 1H), 6.86 (t, J=2.0 Hz, 1H), 6.74 (s, 1H), 4.94 (td, J=4.8, 1.9 Hz, 2H), 4.91-4.83 (m, 1H), 4.77 (td, J=4.7, 1.9 Hz, 2H), 4.25-4.11 (m, 1H), 3.97 (br s, 2H), 3.22 (br d, J=9.4 Hz, 1H), 3.05 (br d, J=11.2 Hz, 1H), 2.81-2.64 (m, 5H), 2.43-2.33 (m, 2H), 2.26-2.11 (m, 4H), 2.04 (q, J=10.7 Hz, 2H), 1.98-1.84 (m, 2H), 1.81-1.72 (m, 1H), 1.46 (br d, J=9.8 Hz, 1H).

### Example 927

### N—((R)-1-(4-amino-7-(5-oxo-2,5-dihydrofuran-3-yl)pyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide, TFA salt

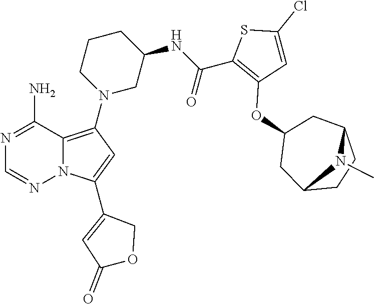 [SMILES: CN1[C@@H]2CC[C@H]1C[C@H](Oc1cc(Cl)sc1C(=O)N[C@@H]1CCCN(c3cc(C4=CC(=O)OC4)n4ncnc(N)c34)C1)C2]

### Example 927A: 4-(4,4,5,5-tetramethyl-1,3,2-dioxaborolan-2-yl)furan-2(5H)-one

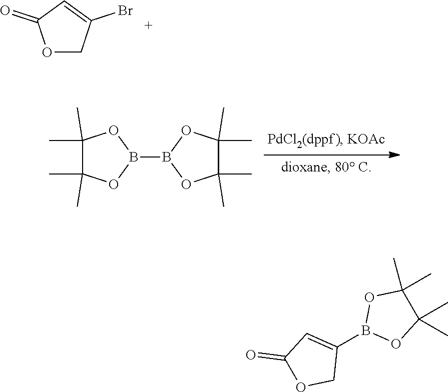

To a solution of 4-bromofuran-2-one (600 mg, 3.68 mmol) in dioxane (15 mL) was added bis(pinacolato)diboron (1.12 g, 4.42 mmol) followed by addition of potassium acetate (723 mg, 7.36 mmol) and PdCl₂(dppf) (269 mg, 0.368 mmol) at rt. The reaction was stirred under N₂ at 80° C. for 4 h. The crude solution was used in the next step.

### Example 927B: tert-butyl (R)-(1-(4-amino-7-(5-oxo-2,5-dihydrofuran-3-yl)pyrrolo[2,1-f][1,2,4]triazin-5-yl)piperidin-3-yl)carbamate

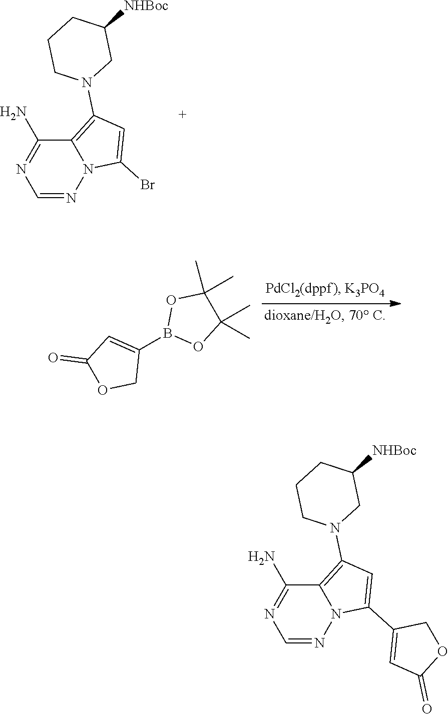 [SMILES: CC(C)(C)OC(=O)N[C@@H]1CCCN(c2cc(Br)n3ncnc(N)c23)C1 ; "+" ; CC1(C)OB(C2=CC(=O)OC2)OC1(C)C ; CC(C)(C)OC(=O)N[C@@H]1CCCN(c2cc(C3=CC(=O)OC3)n3ncnc(N)c23)C1]

To a solution of Intermediate 29 (200 mg, 0.486 mmol) in dioxane (10 mL) was added Example 927A (204 mg, 0.973 mmol) (4 mL reaction solution from step 1) followed by addition of K₃PO₄ (206 mg, 0.973 mmol) and PdCl₂(dppf) (35.6 mg, 0.049 mmol) at rt. The reaction was stirred under N₂ at 70° C. for 4 h. The reaction was cooled and diluted with EtOAc, washed with H₂O and brine. The organic phase was dried over Na₂SO₄, filtered and concentrated. The crude product was purified by normal phase chromatography to afford product (95 mg, 47%). MS (ESI) m/z 415 [M+H]⁺; ¹H NMR (500 MHz, CDCl₃) δ 7.95 (s, 1H), 7.93-7.86 (m, 1H), 7.56-7.47 (m, 1H), 7.45-7.36 (m, 1H), 6.74 (s, 1H), 6.61 (s, 1H), 5.37 (d, J=1.7 Hz, 2H), 3.81 (br s, 1H), 3.42-3.17 (m, 1H), 3.04 (br s, 1H), 2.91-2.23 (m, 2H), 2.05-1.96 (m, 1H), 1.93-1.85 (m, 1H), 1.82-1.73 (m, 1H), 1.57 (br s, 1H), 1.45 (s, 9H).

### Example 927

The example was synthesized from Example 927B following procedures analogous to those described in previous examples using the appropriate intermediates. LC-MS Method B: ESI m/z 598 (M+H)⁺, RT=1.15 min; ¹H NMR (500 MHz, DMSO-d₆) δ 10.02 (br s, 1H), 8.55 (br s, 1H), 8.04 (s, 1H), 7.57 (s, 1H), 7.18 (s, 1H), 6.67 (t, J=1.7 Hz, 1H), 5.40 (d, J=1.1 Hz, 2H), 4.95-4.80 (m, 1H), 4.26-4.11 (m, 1H), 3.97 (br s, 2H), 3.18 (br d, J=7.8 Hz, 1H), 3.08-2.99 (m, 1H), 2.70 (d, J=5.0 Hz, 3H), 2.82-2.65 (m, 2H), 2.42-2.34 (m, 2H), 2.22-2.10 (m, 4H), 2.08-1.99 (m, 2H), 1.96-1.85 (m, 2H), 1.83-1.74 (m, 1H), 1.49 (br d, J=1.0 Hz, 1H).

### Example 928

### N-((3R)-1-(4-amino-7-(5-(1-hydroxyethyl)thiophen-3-yl)pyrrolo[2,1-f][1,2,4]triazin-5-yl)piperidin-3-yl)-2-methyl-4-(((1R,3s,5S)-8-methyl-8-azabicyclo[3.2.1]octan-3-yl)oxy)thiazole-5-carboxamide, TFA salt

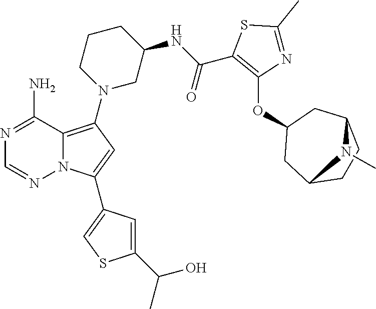 [SMILES: Cc1nc(O[C@@H]2C[C@H]3CC[C@@H](C2)N3C)c(C(=O)N[C@@H]2CCCN(c3cc(-c4csc(C(C)O)c4)n4ncnc(N)c34)C2)s1]

### Example 928A: tert-butyl (R)-(1-(7-(5-acetylthiophen-3-yl)-4-aminopyrrolo[2,1-f][1,2,4]triazin-5-yl)piperidin-3-yl)carbamate

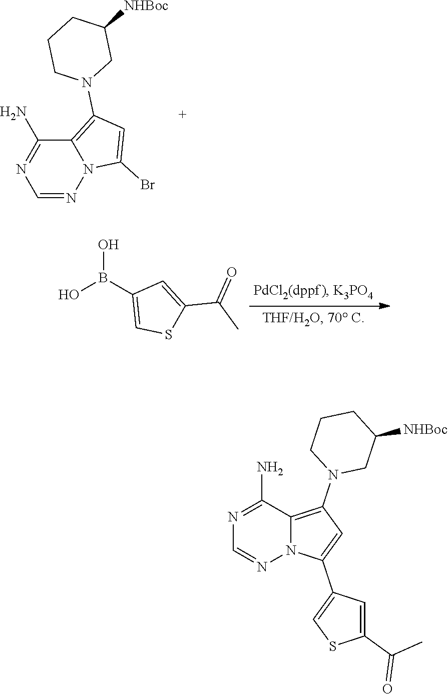 [SMILES: CC(C)(C)OC(=O)N[C@@H]1CCCN(c2cc(Br)n3ncnc(N)c23)C1 ; "+" ; CC(=O)c1cc(B(O)O)cs1 ; CC(=O)c1cc(-c2cc(N3CCC[C@@H](NC(=O)OC(C)(C)C)C3)c3c(N)ncnn23)cs1]

To a solution of Intermediate 29 (200 mg, 0.486 mmol) in THF (10 mL) and H₂O (1 mL) was added 5-acetyl-3-thienylboronic acid (99 mg, 0.584 mmol) followed by addition of K₃PO₄ (258 mg, 1.216 mmol) and PdCl₂(dppf) (35.6 mg, 0.049 mmol) at rt. The reaction was stirred under N₂ at 85° C. for 3 h. The reaction was diluted with EtOAc. The organic phase was separated and filtered through Celite. The solvent was removed. The crude product was purified by normal phase chromatography to afford the product (212 mg, 95%). MS (ESI) m/z 457 [M+H]; ¹H NMR (500 MHz, CDCl₃) δ 8.44 (d, J=1.4 Hz, 1H), 8.20 (d, J=1.4 Hz, 1H), 7.91 (s, 1H), 6.74 (s, 1H), 4.66 (br s, 1H), 3.84 (br s, 1H), 3.49-3.19 (m, 1H), 3.16-2.94 (m, 1H), 2.90-2.69 (m, 1H), 2.63 (s, 3H), 2.02-1.69 (m, 4H), 1.45 (s, 9H).

### Example 928B: tert-butyl ((3R)-1-(4-amino-7-(5-(1-hydroxyethyl)thiophen-3-yl)pyrrolo[2,1-f][1,2,4]triazin-5-yl)piperidin-3-yl)carbamate

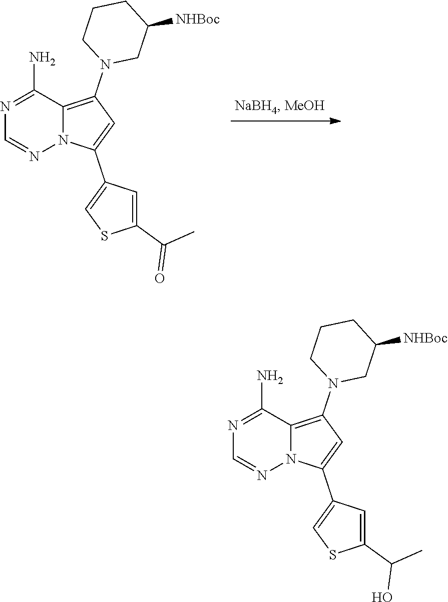 [SMILES: CC(=O)c1cc(-c2cc(N3CCC[C@@H](NC(=O)OC(C)(C)C)C3)c3c(N)ncnn23)cs1 ; CC(O)c1cc(-c2cc(N3CCC[C@@H](NC(=O)OC(C)(C)C)C3)c3c(N)ncnn23)cs1]

To a solution of Example 928A (170 mg, 0.372 mmol) in MeOH (5 mL) was added NaBH₄ (42.3 mg, 1.12 mmol) at 0° C. The reaction was stirred under N₂ at 0° C. for 2 h. The solvent was removed. The residue was suspended in EtOAc and filtered through a plug of silica gel. The solvent was removed from filtrate to give desired product (152 mg, 89%), which was used without further purification. MS (ESI) m/z 459 [M+H]⁺.

### Example 928

Example 928 was synthesized from Example 928B following the procedure analogous to those described in previous examples. LC-MS Method B: ESI m/z 623 (M+H)⁺, RT=1.06 min; ¹H NMR (500 MHz, CD₃OD) δ 8.23 (t, J=1.2 Hz, 1H), 7.96 (d, J=0.6 Hz, 1H), 7.59 (s, 1H), 7.12 (d, J=1.1 Hz, 1H), 5.50-5.38 (m, 1H), 5.12 (q, J=6.5 Hz, 1H), 4.30-4.21 (m, 1H), 4.05 (br s, 2H), 3.45-3.37 (m, 1H), 3.27-3.21 (m, 1H), 3.09-3.00 (m, 1H), 2.98-2.89 (m, 1H), 2.84 (s, 3H), 2.66 (s, 3H), 2.64-2.57 (m, 2H), 2.47-2.39 (m, 2H), 2.26-2.15 (m, 4H), 2.10 (br dd, J=12.5, 4.4 Hz, 1H), 2.02-1.93 (m, 2H), 1.69-1.61 (m, 1H), 1.60 (d, J=6.5 Hz, 3H).

### Example 929

### N—((R)-1-(4-amino-7-(3-fluoro-4-(1H-tetrazol-1-yl)phenyl)pyrrolo[2,1-f][11,2,4]triazin-5-yl)piperidin-3-yl)-4-(((1R,3s,5S)-8-(3-hydroxycyclobutyl)-8-azabicyclo[3.2.1]octan-3-yl)oxy)-2-methylthiazole-5-carboxamide TFA salt

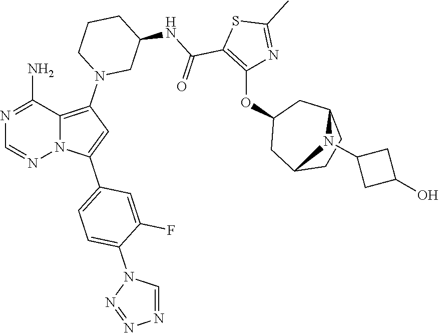 [SMILES: Cc1nc(O[C@@H]2C[C@H]3CC[C@@H](C2)N3C2CC(O)C2)c(C(=O)N[C@@H]2CCCN(c3cc(-c4ccc(-n5cnnn5)c(F)c4)n4ncnc(N)c34)C2)s1]

Example 929 was synthesized by following the procedure analogous as described in previous examples. LC-MS Method B: ESI m/z 715 (M+H)⁺, RT=0.71 min; ¹H NMR (500 MHz, CD₃CN) δ 9.37 (d, J=2.1 Hz, 1H), 8.20 (dd, J=12.5, 1.8 Hz, 1H), 8.11-8.05 (m, 1H), 8.03-7.97 (m, 1H), 7.90 (s, 1H), 7.68 (br s, 1H), 7.19 (s, 1H), 7.11 (br d, J=5.7 Hz, 1H), 5.44-5.29 (m, 1H), 4.26 (dt, J=7.3, 3.6 Hz, 1H), 4.02 (br s, 2H), 3.27 (br d, J=8.7 Hz, 1H), 3.22-3.12 (m, 3H), 3.04 (br t, J=6.8 Hz, 4H), 2.62-2.60 (m, 3H), 2.53-2.47 (m, 2H), 2.38-2.30 (m, 4H), 2.17 (s, 3H), 2.07 (br d, J=9.3 Hz, 2H), 1.96-1.90 (m, 2H), 1.70 (br d, J=7.1 Hz, 1H).

### Example 930

### 4-(((5S,8r)-1-azaspiro[4.5]decan-8-yl)oxy)-N—((R)-1-(4-amino-7-(4-carbamoyl-3-fluorophenyl)pyrrolo[2,1-f][1,2,4]triazin-5-yl)piperidin-3-yl)-2-methylthiazole-5-carboxamide, TFA salt

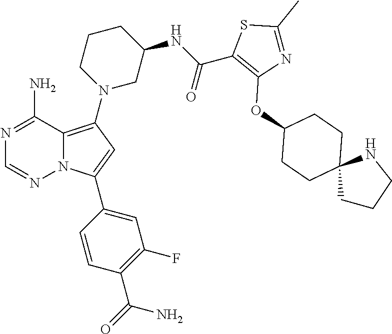 [SMILES: Cc1nc(O[C@H]2CC[C@]3(CCCN3)CC2)c(C(=O)N[C@@H]2CCCN(c3cc(-c4ccc(C(N)=O)c(F)c4)n4ncnc(N)c34)C2)s1]

### Example 930A: tert-butyl (5r,8s)-8-hydroxy-1-azaspiro[4.5]decane-1-carboxylate

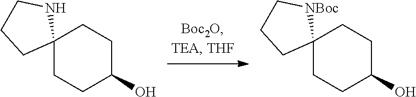 [SMILES: O[C@H]1CC[C@@]2(CCCN2)CC1 ; CC(C)(C)OC(=O)N1CCC[C@]12CC[C@@H](O)CC2]

To a suspension of trans-1-azaspiro[4.5]decan-8-ol (100 mg, 0.522 mmol) in THF (2 mL) was added TEA (0.109 mL, 0.782 mmol) followed by the addition of Boc₂O (0.145 mL, 0.626 mmol) at rt. Then 0.3 mL MeOH was added to increase solubility. The reaction was stirred under N₂ at rt for 4 h. The solvent was removed. The crude product was purified by normal phase chromatography to provide the product (138 mg, 100%). MS (ESI) m/z 200 [M−55]⁺; ¹H NMR (500 MHz, CDCl₃) δ 3.83-3.53 (m, 1H), 3.50-3.30 (m, 2H), 2.63 (br s, 1H), 1.97-1.82 (m, 4H), 1.80-1.65 (m, 4H), 1.45 (br s, 9H), 1.40-1.30 (m, 4H).

### Example 930B: ethyl 4-(((5s,8r)-1-(tert-butoxycarbonyl)-1-azaspiro[4.5]decan-8-yl)oxy)-2-methylthiazole-5-carboxylate

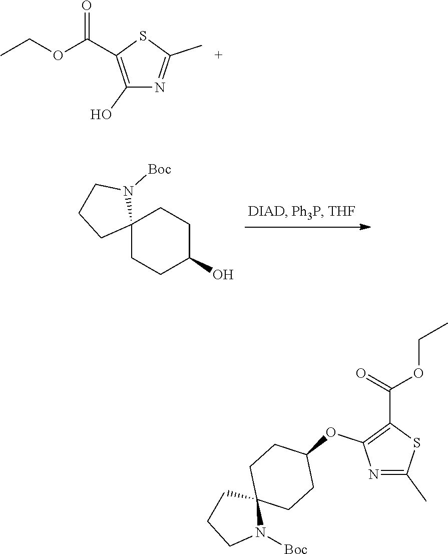

To a solution of Example 930A (138 mg, 0.540 mmol) in THF (5 mL) was added ethyl 4-hydroxy-2-methylthiazole-5-carboxylate (84 mg, 0.450 mmol) followed by addition of Ph₃P (177 mg, 0.676 mmol) and DIAD (0.131 mL, 0.676 mmol) at rt. The reaction was stirred under N₂ at rt for 3.5 h. The solvent was removed. The crude product was purified by normal phase chromatography to provide the product (190 mg, 99%). MS (ESI) m/z 425 [M+H]⁺; ¹H NMR (500 MHz, CDCl₃) δ 5.22-4.70 (m, 1H), 4.48-4.13 (m, 2H), 3.57-3.28 (m, 2H), 3.14-2.67 (m, 2H), 2.55 (s, 3H), 2.37-2.02 (m, 2H), 1.87 (br s, 2H), 1.69 (quin, J=6.8 Hz, 2H), 1.60 (br t, J=14.1 Hz, 2H), 1.42 (br s, 9H), 1.36-1.26 (m, 3H), 1.25-1.15 (m, 2H).

### Example 930C: 4-(((5s,8r)-1-(tert-butoxycarbonyl)-1-azaspiro[4.5]decan-8-yl)oxy)-2-methylthiazole-5-carboxylic acid, Li salt

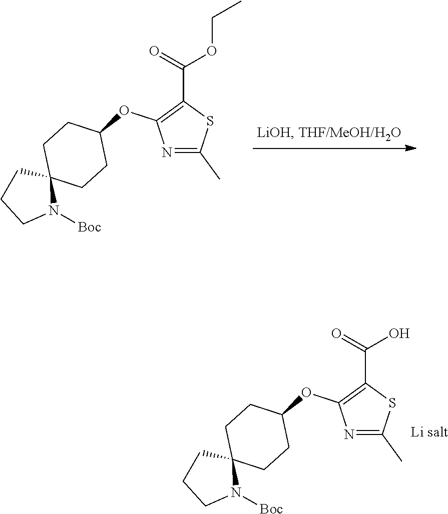 [SMILES: CCOC(=O)c1sc(C)nc1O[C@H]1CC[C@]2(CCCN2C(=O)OC(C)(C)C)CC1 ; Cc1nc(O[C@H]2CC[C@]3(CCCN3C(=O)OC(C)(C)C)CC2)c(C(=O)O)s1]

To a solution of Example 930B (190 mg, 0.448 mmol) in THF (2 mL)/MeOH (1 mL)/H₂O (1 mL) was added LiOH (32.2 mg, 1.34 mmol) at rt. The reaction was stirred under N₂ at rt for 5 h. The solvent was removed to leave a white solid, which was used as is in the following step (185 mg, 103%). MS (ESI) m/z 397 [M+H]⁺.

### Example 930D: tert-butyl (5S,8r)-8-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)-1-azaspiro[4.5]decane-1-carboxylate

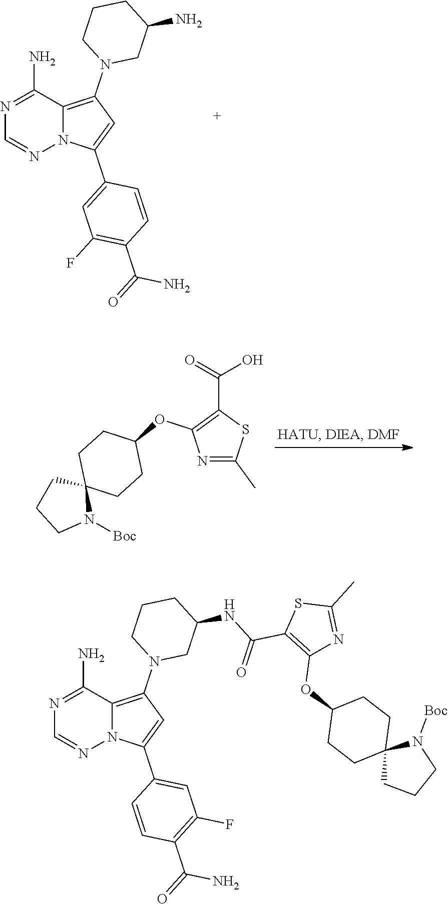 [SMILES: NC(=O)c1ccc(-c2cc(N3CCC[C@@H](N)C3)c3c(N)ncnn23)cc1F ; "+" ; Cc1nc(O[C@H]2CC[C@]3(CCCN3C(=O)OC(C)(C)C)CC2)c(C(=O)O)s1 ; Cc1nc(O[C@H]2CC[C@]3(CCCN3C(=O)OC(C)(C)C)CC2)c(C(=O)N[C@@H]2CCCN(c3cc(-c4ccc(C(N)=O)c(F)c4)n4ncnc(N)c34)C2)s1]

To a solution of Example 139D, HCl salt (50 mg, 0.113 mmol) in DMF (3 mL) was added Example 930C (44.8 mg, 0.113 mmol) followed by the addition of DIEA (0.099 mL, 0.565 mmol) and HATU (51.6 mg, 0.136 mmol) at it. The reaction was stirred under N₂ at rt for 30 min. The crude product was purified by reverse phase chromatography to give desired product (42 mg, 50%). MS (ESI) m/z 748[M+H]⁺.

### Example 930

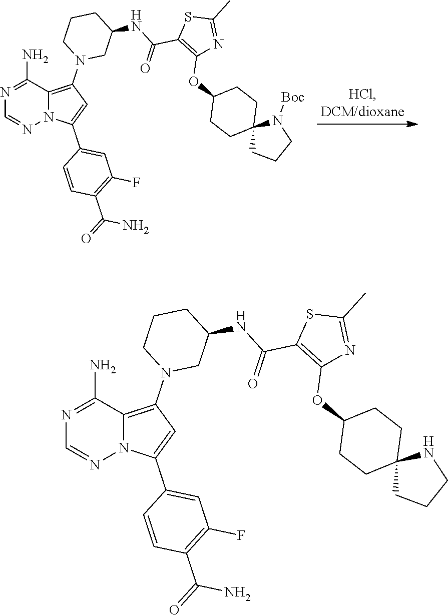 [SMILES: Cc1nc(O[C@H]2CC[C@]3(CCCN3C(=O)OC(C)(C)C)CC2)c(C(=O)N[C@@H]2CCCN(c3cc(-c4ccc(C(N)=O)c(F)c4)n4ncnc(N)c34)C2)s1 ; Cc1nc(O[C@H]2CC[C@]3(CCCN3)CC2)c(C(=O)N[C@@H]2CCCN(c3cc(-c4ccc(C(N)=O)c(F)c4)n4ncnc(N)c34)C2)s1]

To a solution of Example 930D (42 mg, 0.056 mmol) in DCM (2 mL) was added 4 M HCl in dioxane (0.28 mL, 1.12 mmol) at rt. A white suspension was formed. The reaction was stirred under N₂ at rt for 30 min. The solvent was removed. The solid was dissolved in DCM/MeOH and evaporated twice. Purification by reverse phase chromatography provide the product (41 mg, 100%). LC-MS Method B: ESI m/z 648 (M+H)⁺, RT=1.18 min; ¹H NMR (500 MHz, DMSO-d₆) δ 8.85 (br s, 2H), 8.08 (br d, J=12.1 Hz, 1H), 8.00-7.92 (m, 2H), 7.83-7.75 (m, 1H), 7.69-7.58 (m, 2H), 7.21 (s, 2H), 5.02 (br s, 1H), 4.20-4.13 (m, 1H), 3.38-3.23 (m, 3H), 3.17-3.04 (m, 1H), 2.94-2.69 (m, 2H), 2.61 (s, 3H), 2.03-1.75 (m, 15H), 1.60-1.45 (m, 1H).

### Example 931

### N—((R)-1-(4-amino-7-(4-carbamoyl-3-fluorophenyl)pyrrolo[2,1-f][1,2,4]triazin-5-yl)piperidin-3-yl)-2-methyl-4-(((5S,8r)-1-methyl-1-azaspiro[4.5]decan-8-yl)oxy)thiazole-5-carboxamide, TFA salt

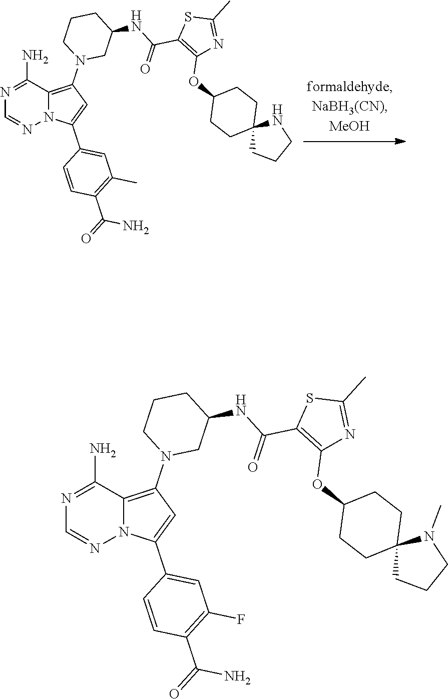 [SMILES: Cc1nc(O[C@H]2CC[C@]3(CCCN3)CC2)c(C(=O)N[C@@H]2CCCN(c3cc(-c4ccc(C(N)=O)c(C)c4)n4ncnc(N)c34)C2)s1 ; Cc1nc(O[C@H]2CC[C@]3(CCCN3C)CC2)c(C(=O)N[C@@H]2CCCN(c3cc(-c4ccc(C(N)=O)c(F)c4)n4ncnc(N)c34)C2)s1]

To a solution of Example 930 (30 mg, 0.042 mmol) in MeOH (1 mL) was added formaldehyde (0.015 mL, 0.208 mmol) followed by addition of acetic acid (0.024 mL, 0.416 mmol) and sodium cyanoborohydride (7.85 mg, 0.125 mmol) at rt. The reaction was stirred under N₂ at rt for 1 h, 1.5 M K₂HPO₄ solution (0.2 mL) was added, and most solvent was removed. Purification by reverse phase chromatography provided Example 931 (13.5 mg, 35.0%). LC-MS Method B: ESI m/z 662 (M+H)⁺, RT=1.11 min; ¹H NMR (500 MHz, DMSO-d₆) δ 8.09 (br d, J=12.0 Hz, 1H), 7.99-7.92 (m, 2H), 7.79 (t, J=7.9 Hz, 1H), 7.69-7.58 (m, 2H), 7.20 (s, 1H), 5.11 (br s, 1H), 4.17 (br dd, J=8.6, 4.2 Hz, 1H), 3.36-3.26 (m, 1H), 3.16-3.05 (m, 1H), 2.95-2.72 (m, 2H), 2.66 (br s, 2H), 2.61 (s, 3H), 2.54 (br d, J=1.5 Hz, 3H), 2.37-2.17 (m, 3H), 2.16-1.70 (m, 10H), 1.68-1.43 (m, 3H).

### Example 932

### N—((R)-1-(4-amino-7-(3-fluoro-4-(methylcarbamoyl)phenyl)pyrrolo[2,1-f][1,2,4]triazin-5-yl)piperidin-3-yl)-4-(((1s,4S)-4-(dimethylamino)cyclohexyl)oxy)-2-methylthiazole-5-carboxamide

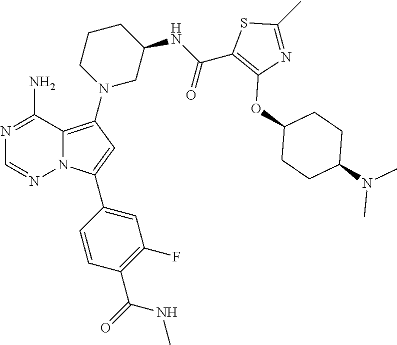 [SMILES: CNC(=O)c1ccc(-c2cc(N3CCC[C@@H](NC(=O)c4sc(C)nc4O[C@H]4CC[C@@H](N(C)C)CC4)C3)c3c(N)ncnn23)cc1F]

Example 932 was synthesized by following the procedure analogous as described in previous examples. LC-MS Method B: ESI m/z 650 (M+H)⁺, RT=1.11 min; ¹H NMR (500 MHz, DMSO-d₆) δ 8.25-8.17 (m, 1H), 8.12 (dd, J=12.8, 1.3 Hz, 1H), 7.99 (dd, J=8.2, 1.5 Hz, 1H), 7.92 (s, 1H), 7.70 (t, J=8.2 Hz, 1H), 7.20 (s, 1H), 5.12 (br d, J=1.0 Hz, 1H), 4.22-4.16 (m, 1H), 3.24-3.19 (m, 21H), 3.02-2.84 (m, 3H), 2.80 (d, J=4.6 Hz, 3H), 2.60 (s, 3H), 2.15-2.08 (m, 2H), 2.02-1.95 (m, 2H), 1.91 (s, 6H), 1.86-1.79 (m, 2H), 1.69-1.54 (m, 4H), 1.50-1.41 (m, 2H).

### Example 933

### Preparation of N—((R)-1-(4-Amino-7-(4-(4-(2-cyanoethyl)-1H-1,2,3-triazol-1-yl)-3-fluorophenyl)pyrrolo[2,1-f][1,2,4]triazin-5-yl)piperidin-3-yl)-5-methyl-3-(((1R,3s,5S)-8-methyl-8-azabicyclo[3.2.1]octan-3-yl)oxy)thiophene-2-carboxamide

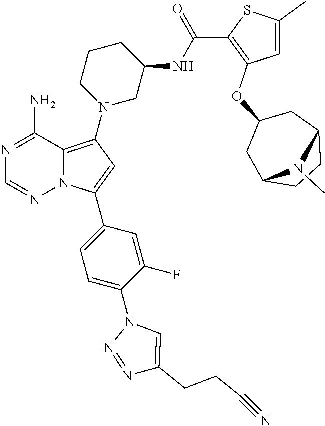 [SMILES: Cc1cc(O[C@@H]2C[C@H]3CC[C@@H](C2)N3C)c(C(=O)N[C@@H]2CCCN(c3cc(-c4ccc(-n5cc(CCC#N)nn5)c(F)c4)n4ncnc(N)c34)C2)s1]

### Example 933A. N—((R)-1-(4-Amino-7-bromopyrrolo[2,1-f][1,2,4]triazin-5-yl)piperidin-3-yl)-5-methyl-3-(((1R,3s,5S)-8-methyl-8-azabicyclo[3.2.1]octan-3-yl)oxy)thiophene-2-carboxamide

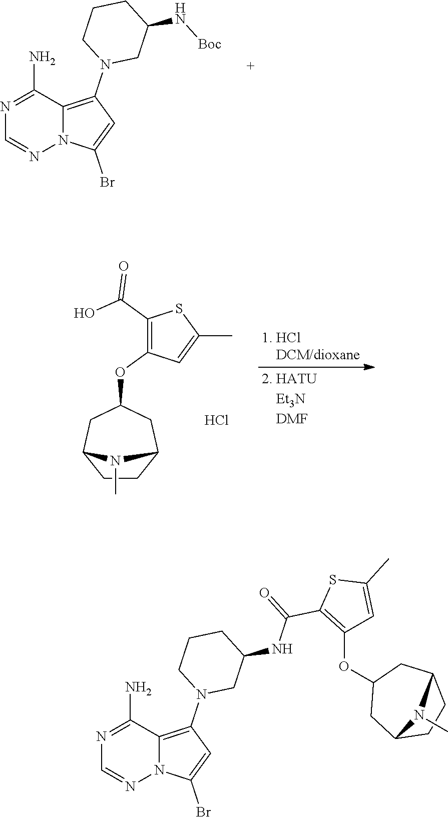 [SMILES: CC(C)(C)OC(=O)N[C@@H]1CCCN(c2cc(Br)n3ncnc(N)c23)C1 ; Cc1cc(O[C@@H]2C[C@H]3CC[C@@H](C2)N3C)c(C(=O)O)s1 ; Cl ; Cc1cc(OC2C[C@H]3CC[C@@H](C2)N3C)c(C(=O)N[C@@H]2CCCN(c3cc(Br)n4ncnc(N)c34)C2)s1]

A stirring solution of Intermediate 29 (534 mg, 1.298 mmol) in dichloromethane (5 mL) was treated with 4M HCl in dioxane (3 mL). The reaction was stirred at rt for 1 h, at which point it was judged to be complete by LCMS. The mixture was concentrated 3× from dichloromethane, then the residue was taken up in DMF (2 mL). The solution was treated with Intermediate 66, HCl salt (578 mg, 1.818 mmol), triethylamine (1.1 mL, 7.79 mmol), and then HATU (543 mg, 1.43 mmol), and the reaction was stirred at rt for 3 h. The mixture was taken up in ethyl acetate (30 mL), and the turbid solution was washed once with water, twice with 10% lithium chloride, and once with brine, then dried over sodium sulfate and concentrated in vacuo. The residue was chromatographed via MPLC (silica gel column, 0% to 15% [7M ammonia in methanol]/dichloromethane gradient) to afford Example 933A (136 mg, 18% yield). MS: [M+H]⁺=575.9.

### Example 933B. N—((R)-1-(4-Amino-7-(4-bromo-3-fluorophenyl)pyrrolo[2,1-f][1,2,4]triazin-5-yl)piperidin-3-yl)-5-methyl-3-(((1R,3s,5S)-8-methyl-8-azabicyclo[3.2.1]octan-3-yl)oxy)thiophene-2-carboxamide

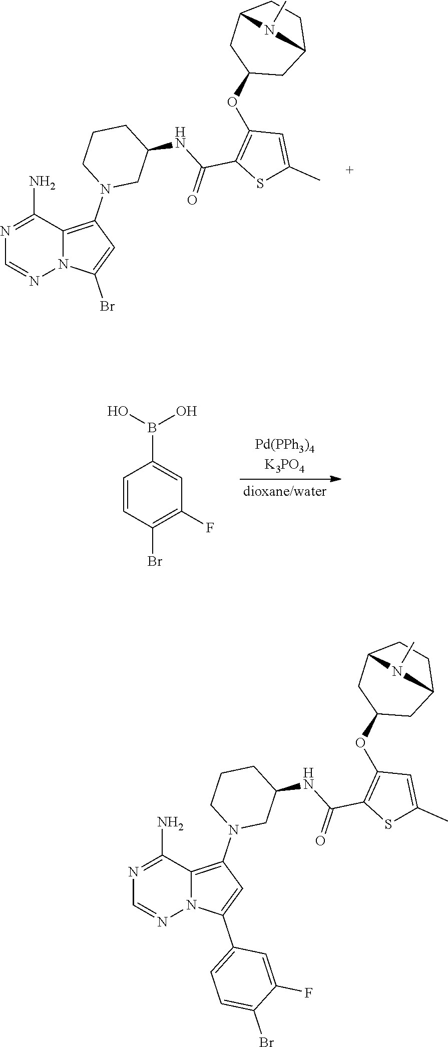 [SMILES: Cc1cc(O[C@@H]2C[C@H]3CC[C@@H](C2)N3C)c(C(=O)N[C@@H]2CCCN(c3cc(Br)n4ncnc(N)c34)C2)s1 ; OB(O)c1ccc(Br)c(F)c1 ; Cc1cc(O[C@@H]2C[C@H]3CC[C@@H](C2)N3C)c(C(=O)N[C@@H]2CCCN(c3cc(-c4ccc(Br)c(F)c4)n4ncnc(N)c34)C2)s1]

In a 40 mL vial, a mixture of Example 933A (136 mg, 0.237 mmol), (4-bromo-3-fluorophenyl)boronic acid (51.8 mg, 0.24 mmol), tetrakis(triphenylphosphine)palladium(0) (41.0 mg, 0.04 mmol), potassium phosphate, tribasic (201 mg, 0.95 mmol) in 2:1dioxane/water (3 mL) was degassed in a sonicator with bubbling nitrogen for 5 minutes. The vial was sealed, and the reaction was stirred at 110° C. for 1.5 h. The mixture was diluted with ethyl acetate (25 mL). The turbid solution was washed twice with water and once with brine, and the organic phase was dried over sodium sulfate and concentrated in vacuo. The residue was chromatographed via MPLC (silica gel column, 0-5% [7M ammonia in methanol]/dichloromethane gradient) to yield Example 933B (110 mg, 70% yield). MS: [M+H]⁺=668.2/669.9.

### Example 933

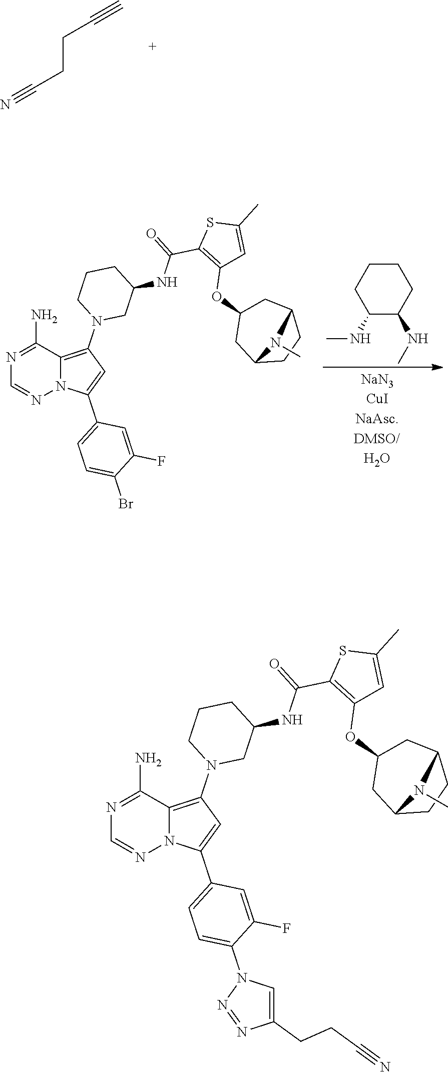

In a scintillation vial, a mixture of pent-4-ynenitrile (4.68 mg, 0.059 mmol), Example 933B (33 mg, 0.05 mmol), (1R,2R)—N1,N2-dimethylcyclohexane-1,2-diamine (0.02 mL, 0.1 mmol), and sodium azide (3.85 mg, 0.06 mmol) in DMSO/water (4:1) (1 mL) was treated with copper(I) iodide (11 mg, 0.06 mmol) and sodium ascorbate (12 mg, 0.06 mmol). The vial was sealed, and the reaction was stirred at 80° C. for 18 h. The mixture was diluted with DMF, filtered and purified by preparative HPLC to afford Example 933 (7.4 mg, 21% yield). LCMS Method B: RT=1.26 min, [M+H]⁺=710.6; ¹H NMR (500 MHz, DMSO-d₆) δ 8.51 (d, J=1.8 Hz, 1H), 8.37-8.31 (m, 1H), 8.16 (dd, J=8.4, 1.1 Hz, 1H), 8.01 (s, 1H), 7.94 (t, J=8.4 Hz, 1H), 7.32 (s, 1H), 7.09 (s, 1H), 4.93-4.76 (m, 1H), 4.21-4.12 (m, 1H), 3.96 (br d, J=1.4 Hz, 2H), 3.57-3.48 (m, 1H), 3.32-3.22 (m, 1H), 3.11-3.06 (m, 2H), 2.97-2.92 (m, 2H), 2.90-2.80 (m, 1H), 2.70 (br d, J=2.9 Hz, 3H), 2.42 (s, 3H), 2.41-2.33 (m, 2H), 2.27-1.77 (m, 10H), 1.57-1.40 (m, 1H).

### Example 934

### Preparation of 4-(((1s,4S)-4-Amino-4-methylcyclohexyl)oxy)-N—((R)-1-(4-amino-7-(3-fluoro-4-(1H-tetrazol-1-yl)phenyl)pyrrolo[2,1-f][1,2,4]triazin-5-yl)piperidin-3-yl)-2-methylthiazole-5-carboxamide

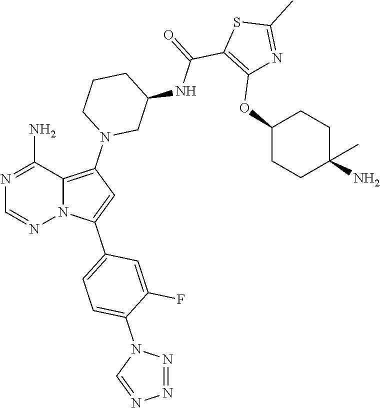 [SMILES: Cc1nc(O[C@H]2CC[C@@](C)(N)CC2)c(C(=O)N[C@@H]2CCCN(c3cc(-c4ccc(-n5cnnn5)c(F)c4)n4ncnc(N)c34)C2)s1]

### Example 934A. Ethyl 4-(((1s,4s)-4-((tert-butoxycarbonyl)amino)-4-methylcyclohexyl)oxy)-2-methylthiazole-5-carboxylate

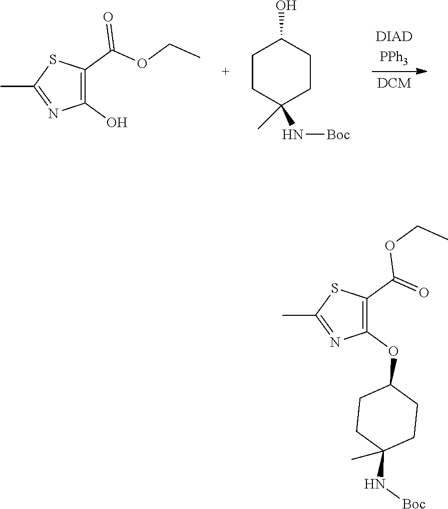 [SMILES: CCOC(=O)c1sc(C)nc1O ; CC(C)(C)OC(=O)N[C@]1(C)CC[C@@H](O)CC1 ; CCOC(=O)c1sc(C)nc1O[C@H]1CC[C@@](C)(NC(=O)OC(C)(C)C)CC1]

In a dried 40 mL vial, a stirring mixture of ethyl 4-hydroxy-2-methylthiazole-5-carboxylate (367 mg, 1.96 mmol) and tert-butyl ((1r,4r)-4-hydroxy-1-methylcyclohexyl)carbamate (540 mg, 2.36 mmol) in dichloromethane (10 mL) was treated with triphenylphosphine (1.54 g, 5.89 mmol) then DIAD (0.6 mL, 3.0 mmol). The vial was sealed, and the reaction was stirred at rt for 18 h. The mixture was concentrated in vacuo, and the residue was chromatographed via MPLC (silica gel column, 10% to 100% ethyl acetate/hexanes gradient) to yield Example 934A (562 mg, 72% yield). MS: [M+H]⁺=399.1; ¹H NMR (500 MHz, CDCl₃) δ 5.16 (br s, 1H), 4.43-4.35 (m, 1H), 4.30 (q, J=7.1 Hz, 2H), 2.62 (s, 3H), 1.97-1.75 (m, 8H), 1.47 (s, 9H), 1.40-1.31 (m, 6H).

### Example 934B 4-(((1s,4s)-4-((tert-Butoxycarbonyl)amino)-4-methylcyclohexyl)oxy)-2-methylthiazole-5-carboxylic acid

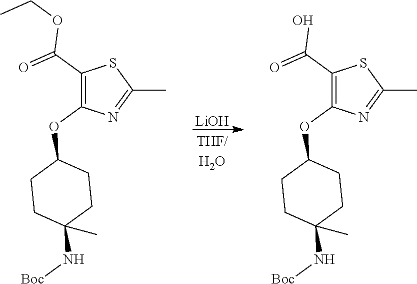

A stirring solution of Example 934A (562 mg, 1.41 mmol) in THF (5 mL) was treated with 0.5 M lithium hydroxide (4.2 mL, 2.1 mmol). The reaction was stirred at 50° C. for 3 hours, then at it for 3 d. The mixture was cooled to 0° C. and treated with 1M HCl (2.2 mL). The mixture was extracted 3× with ethyl acetate, then the combined organic phases were washed once with brine, dried over sodium sulfate, and concentrated in vacuo to yield Example 934B (509 mg, 97% yield) as a colorless solid; ¹H NMR (500 MHz, CDCl₃) δ 5.25-5.20 (m, 1H), 4.38 (br d, J=7.2 Hz, 1H), 2.66 (s, 3H), 2.05-1.86 (m, 6H), 1.82-1.71 (m, 2H), 1.51-1.44 (m, 10H), 1.39 (s, 3H).

### Example 934C. tert-Butyl ((1S,4s)-4-((5-(((R)-1-(4-amino-7-bromopyrrolo[2,1-f][1,2,4]triazin-5-yl)piperidin-3-yl)carbamoyl)-2-methylthiazol-4-yl)oxy)-1-methylcyclohexyl)carbamate

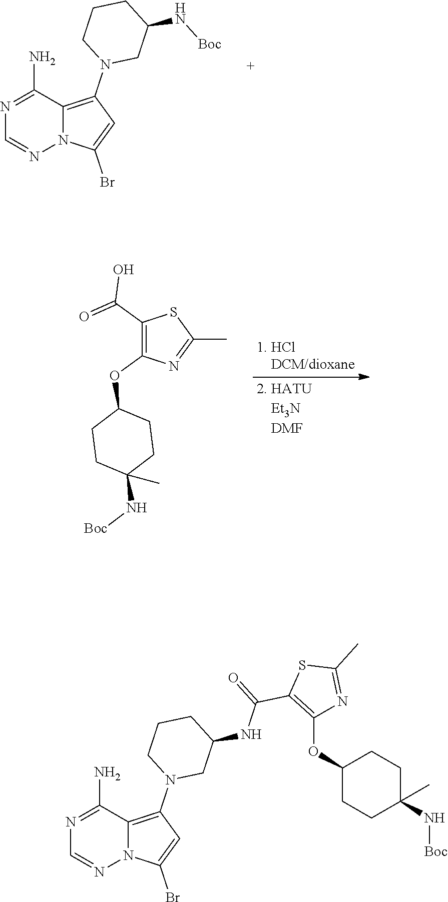 [SMILES: CC(C)(C)OC(=O)N[C@@H]1CCCN(c2cc(Br)n3ncnc(N)c23)C1 ; Cc1nc(O[C@H]2CC[C@@](C)(NC(=O)OC(C)(C)C)CC2)c(C(=O)O)s1 ; Cc1nc(O[C@H]2CC[C@@](C)(NC(=O)OC(C)(C)C)CC2)c(C(=O)N[C@@H]2CCCN(c3cc(Br)n4ncnc(N)c34)C2)s1]

A stirring solution of Intermediate 29 (473 mg, 1.15 mmol) in dichloromethane (5 mL) was treated with 4M HCl in dioxane (3 mL). The reaction was stirred at rt for 1 h. The mixture was concentrated 3× from dichloromethane, then the residue was taken up in DMF (2 mL). The solution was treated with Example 934B (426 mg, 1.15 mmol), triethylamine (0.96 mL, 6.90 mmol), and then HATU (481 mg, 1.27 mmol), and the reaction was stirred at rt for 3 h. The mixture was taken up in ethyl acetate (30 mL), and the turbid solution was washed once with water, twice with 10% lithium chloride, and once with brine, then dried over sodium sulfate and concentrated in vacuo. The residue was chromatographed via MPLC (silica gel column, 30% to 100% ethyl acetate/hexanes gradient) to yield Example 934C (595 mg, 78% yield). MS: [M+H]⁺=663.2/665.3.

### Example 934

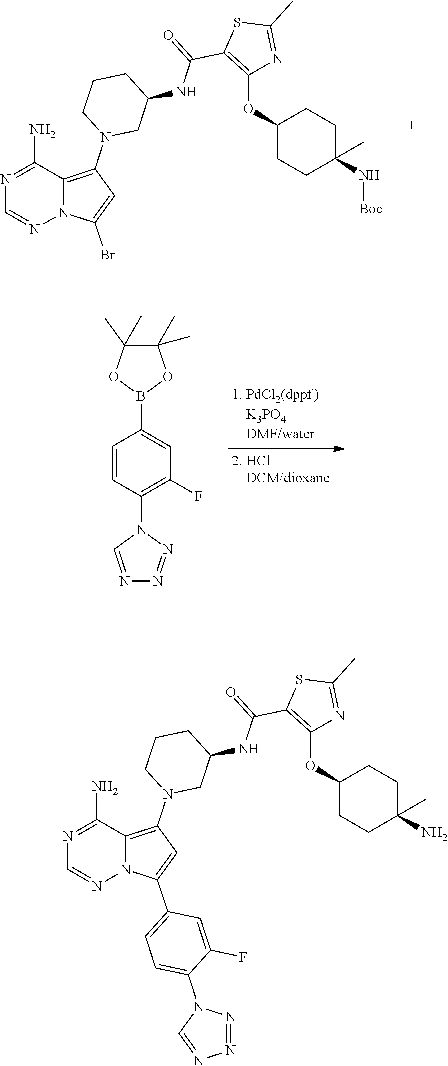 [SMILES: Cc1nc(O[C@H]2CC[C@@](C)(NC(=O)OC(C)(C)C)CC2)c(C(=O)N[C@@H]2CCCN(c3cc(Br)n4ncnc(N)c34)C2)s1 ; CC1(C)OB(c2ccc(-n3cnnn3)c(F)c2)OC1(C)C ; Cc1nc(O[C@H]2CC[C@@](C)(N)CC2)c(C(=O)N[C@@H]2CCCN(c3cc(-c4ccc(-n5cnnn5)c(F)c4)n4ncnc(N)c34)C2)s1]

In a 2-dram vial, a mixture of Example 934C (179 mg, 0.27 mmol), Intermediate 2 (156 mg, 0.54 mmol), PdCl₂(dppf) (20 mg, 0.03 mmol), and potassium phosphate, tribasic (115 mg, 0.54 mmol) in DMF-water (9:1) (5 mL) was degassed in a sonicator with bubbling nitrogen for 5 minutes. The vial was sealed, and the reaction was stirred at 80° C. for 1.5 h. The mixture was filtered, and the filtrate was diluted with ethyl acetate. The turbid solution was washed once with water, twice with 10% lithium chloride, and once with brine, then dried over sodium sulfate and concentrated in vacuo. The residue was purified by flash chromatograph (25% to 100% ethyl acetate/hexanes gradient) to yield tert-butyl ((1S,4s)-4-((5-(((R)-1-(4-amino-7-(3-fluoro-4-(1H-tetrazol-1-yl)phenyl)pyrrolo[2,1-f][1,2,4]triazin-5-yl)piperidin-3-yl)carbamoyl)-2-methylthiazol-4-yl)oxy)-1-methylcyclohexyl)carbamate (138 mg, 0.14 mmol, 53% yield). MS: [M+H]⁺=747.4. The material was taken up in dichloromethane (2 mL), and the solution was treated with 4M HCl in dioxane (1 mL). The mixture was stirred for 1 h, then concentrated in vacuo. The residue was concentrated 3× from dichloromethane, and the residue was dried under vacuum. A 20 mg portion of the material was purified by preparative HPLC to yield Example 934 (5.3 mg, 3% yield); ¹H NMR (500 MHz, DMSO-d6) δ 9.97 (s, 1H), 8.44 (d, J=12.7 Hz, 1H), 8.24 (d, J=8.4 Hz, 1H), 8.00-7.91 (m, 2H), 7.31 (s, 2H), 5.03 (br d, J=6.3 Hz, 1H), 4.25-4.11 (m, 1H), 3.91 (s, 1H), 3.00 (br s, 1H), 2.61 (s, 3H), 2.07-1.93 (m, 3H), 1.91-1.82 (m, 5H), 1.74-1.63 (m, 2H), 1.59-1.45 (m, 3H), 1.44-1.29 (m, 2H), 0.97 (br d, J=1.4 Hz, 3H). LCMS Method B: RT=1.24 min, [M+H]⁺=647.1.

### Example 935

### Preparation of (R)—N-(1-(4-Amino-7-(3-fluoro-4-(1H-tetrazol-1-yl)phenyl)pyrrolo[2,1-f][1,2,4]triazin-5-yl)piperidin-3-yl)-5-chloro-3-((4-hydroxycyclohexyl)oxy)thiophene-2-carboxamide, isomer 2

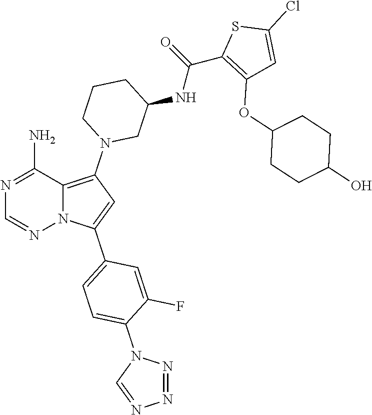 [SMILES: Nc1ncnn2c(-c3ccc(-n4cnnn4)c(F)c3)cc(N3CCC[C@@H](NC(=O)c4sc(Cl)cc4OC4CCC(O)CC4)C3)c12]

### Example 935A. Methyl 5-chloro-3-(4-hydroxycyclohexyl)oxy)thiophene-2-carboxylate

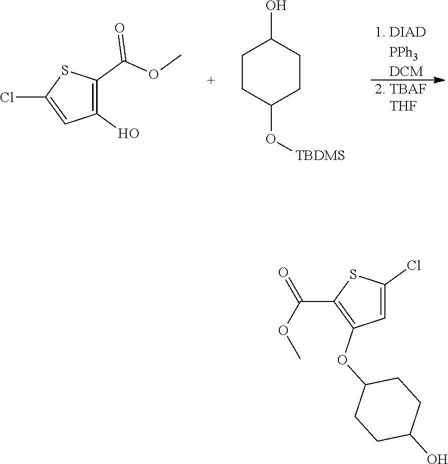

In a dried 40 mL vial, a stirring mixture of methyl 5-chloro-3-hydroxythiophene-2-carboxylate (0.697 g, 3.62 mmol) and 4-((tert-butyldimethylsilyl)oxy)cyclohexan-1-ol (1 g, 4.34 mmol) in dichloromethane (10 mL) was treated with triphenylphosphine (2.85 g, 10.85 mmol), then DIAD (1.1 mL, 5.42 mmol). The vial was sealed, and the reaction was stirred at room temperature for 18 h. The mixture was poured into ethyl acetate, and the solution was washed 3× with water, and once with brine. The organic phase was dried over sodium sulfate and concentrated in vacuo. The residue was chromatographed via MPLC (silica gel column, 0% to 50% ethyl acetate/hexanes gradient). The material was dissolved in THF (5 mL) and treated with TBAF (1M in THF) (5.42 mL, 5.42 mmol). The reaction was stirred at rt for 18 h, then was diluted with ethyl acetate (50 mL), and the turbid solution was washed 3× with water and once with brine, then dried over sodium sulfate and concentrated in vacuo. The residue was chromatographed via MPLC (silica gel column, 0% to 100% ethyl acetate/hexanes gradient to yield Example 935A (0.76 g, 72% yield). MS: [M+H]⁺=290.9.

### Example 935B. 5-Chloro-4-((4-hydroxycyclohexyl)oxy)thiophene-2-carboxylic acid

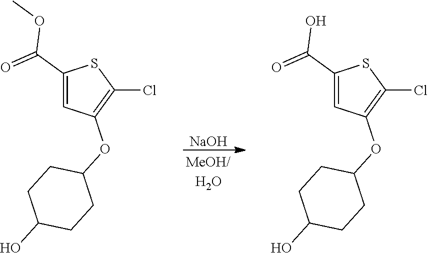 [SMILES: COC(=O)c1cc(OC2CCC(O)CC2)c(Cl)s1 ; "NaOH" ; O=C(O)c1cc(OC2CCC(O)CC2)c(Cl)s1]

A stirring solution of Example 935A (0.76 g, 2.61 mmol) in methanol (10 mL) was treated with 1M NaOH (5 mL, 5.0 mmol). The reaction was stirred at rt for 6 h. Additional 1 M NaOH (1.57 mL, 1.57 mmol) was added, and the reaction was stirred at rt for 2 d. The reaction was heated to 50° C. and stirred for 6 h. The mixture was treated with 1M HCl (1.1 mL, 1.1 mmol), and the mixture was concentrated in vacuo 3× from isopropanol to yield Example 935B. MS: [M+H]⁺=276.9.

### Example 935C. (R)—N-(1-(4-Amino-7-bromopyrrolo[2,1-f][1,2,4]triazin-5-yl)piperidin-3-yl)-5-chloro-3-((4-hydroxycyclohexyl)oxy)thiophene-2-carboxamide

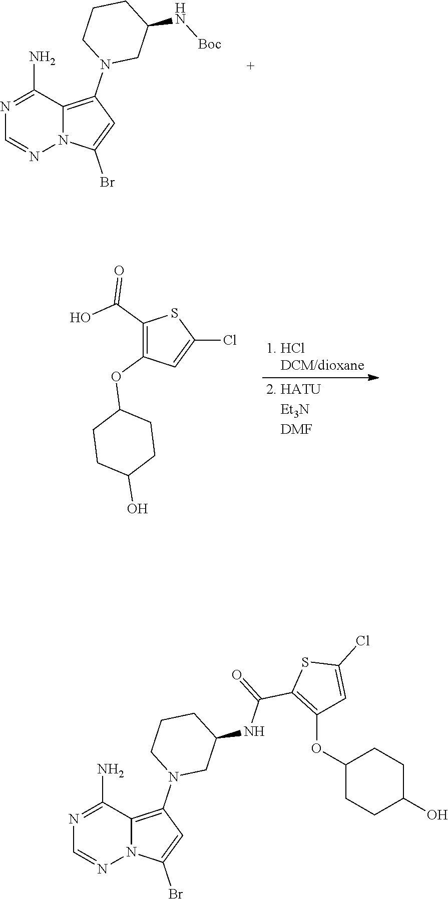 [SMILES: CC(C)(C)OC(=O)N[C@@H]1CCCN(c2cc(Br)n3ncnc(N)c23)C1 ; O=C(O)c1sc(Cl)cc1OC1CCC(O)CC1 ; Nc1ncnn2c(Br)cc(N3CCC[C@@H](NC(=O)c4sc(Cl)cc4OC4CCC(O)CC4)C3)c12]

A stirring solution of Intermediate 29 (871 mg, 2.12 mmol) in dichloromethane (5 mL) was treated with 4M HC in dioxane (3 mL). The reaction was stirred at rt for 1 h, at which point it was judged to be complete by LCMS. The mixture was concentrated 3× from dichloromethane, then the residue was taken up in DMF (2 mL). The solution was treated with Example 935B, triethylamine (1.8 mL, 12.7 mmol), and then HA TU (886 mg, 2.33 mmol), and the reaction was stirred at rt for 3 h. The mixture was taken up in ethyl acetate (30 mL), and the turbid solution was washed once with water, twice with 10% lithium chloride, and once with brine, then dried over sodium sulfate and concentrated in vacuo. The residue was chromatographed via MPLC (silica gel column, 40% to 100% ethyl acetate/hexanes gradient) to yield Example 935C (1.04 g, 86% yield). MS: [M+H]⁺=571.0.

### Example 935

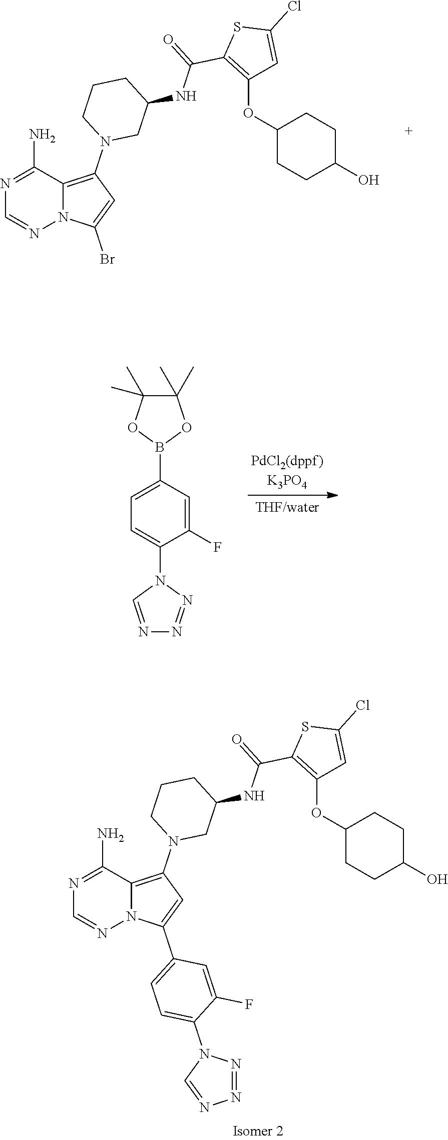 [SMILES: Nc1ncnn2c(Br)cc(N3CCC[C@@H](NC(=O)c4sc(Cl)cc4OC4CCC(O)CC4)C3)c12 ; CC1(C)OB(c2ccc(-n3cnnn3)c(F)c2)OC1(C)C ; Nc1ncnn2c(-c3ccc(-n4cnnn4)c(F)c3)cc(N3CCC[C@@H](NC(=O)c4sc(Cl)cc4OC4CCC(O)CC4)C3)c12]

In a 2-dram vial, a mixture of Example 935C (82 mg, 0.14 mmol), Intermediate 2 (50.1 mg, 0.17 mmol), PdCl₂(dppf) (11 mg, 0.02 mmol), potassium phosphate, tribasic (61.1 mg, 0.29 mmol) in DMF-water (9:1) (2 mL) was degassed with bubbling nitrogen for 10 minutes. The vial was sealed, and the reaction was stirred at 80° C. for 1.5 h. The mixture was diluted with DMF, filtered and purified by preparative HPLC to afford Example 935 (4.5 mg, 4% yield); ¹H NMR (500 MHz, DMSO-d₆) δ 9.97 (s, 1H), 8.45 (dd, J=12.8, 1.7 Hz, 1H), 8.25 (br d, J=8.1 Hz, 1H), 8.21-8.06 (m, 1H), 8.02-7.90 (m, 2H), 7.46-7.36 (m, 2H), 7.35 (s, 1H), 7.02-6.63 (m, 1H), 4.61 (d, J=4.0 Hz, 1H), 4.54-4.44 (m, 1H), 4.30-4.07 (m, 1H), 3.10-2.92 (m, 1H), 2.04 (br dd, J=8.4, 4.2 Hz, 2H), 1.95-1.74 (m, 5H), 1.73-1.43 (m, 3H), 1.40-1.26 (m, 2H). LCMS Method B: RT=1.59 min, [M+H]⁺=653.1.

### Example 936

### Preparation of (R)—N-(1-(7-(1-Acetyl-2,5-dihydro-1H-pyrrol-3-yl)-4-aminopyrrolo[2,1-f][1,2,4]triazin-5-yl)piperidin-3-yl)-5-chloro-3-((4-morpholinocyclohexyl)oxy)thiophene-2-carboxamide, isomer 1

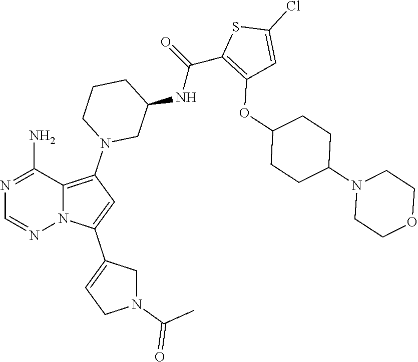 [SMILES: CC(=O)N1CC=C(c2cc(N3CCC[C@@H](NC(=O)c4sc(Cl)cc4OC4CCC(N5CCOCC5)CC4)C3)c3c(N)ncnn23)C1]

### Example 936A. (R)—N-(1-(4-Amino-7-bromopyrrolo[2,1-f][1,2,4]triazin-5-yl)piperidin-3-yl)-5-chloro-3-((4-oxocyclohexyl)oxy)thiophene-2-carboxamide

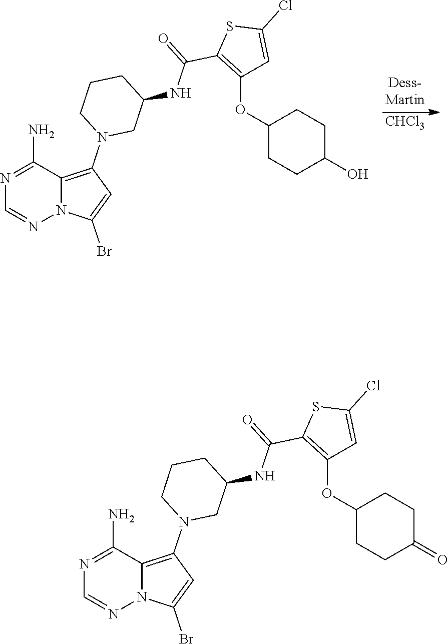 [SMILES: Nc1ncnn2c(Br)cc(N3CCC[C@@H](NC(=O)c4sc(Cl)cc4OC4CCC(O)CC4)C3)c12 ; "CHCl3" ; Nc1ncnn2c(Br)cc(N3CCC[C@@H](NC(=O)c4sc(Cl)cc4OC4CCC(=O)CC4)C3)c12]

A stirring solution of Example 935C (0.96 g, 1.68 mmol) in chloroform (5 mL) was cooled to 0° C. and treated with Dess-Martin periodinane (0.77 g, 1.85 mmol). The reaction was allowed to slowly come to rt and stirred for 18 h. The mixture was diluted with chloroform and filtered, and the solids were washed with chloroform. The combined filtrate was concentrated in vacuo, and the residue was purified via MPLC (silica gel column, 30-100% ethyl acetate/hexanes gradient) to yield Example 936A (416 mg, 44% yield) as an amber solid. MS: [M+H]⁺=567.1/569.0.

### Example 936B. (R)—N-(1-(7-(1-Acetyl-2,5-dihydro-1H-pyrrol-3-yl)-4-aminopyrrolo[2,1-f][1,2,4]triazin-5-yl)piperidin-3-yl)-5-chloro-3-((4-oxocyclohexyl)oxy)thiophene-2-carboxamide

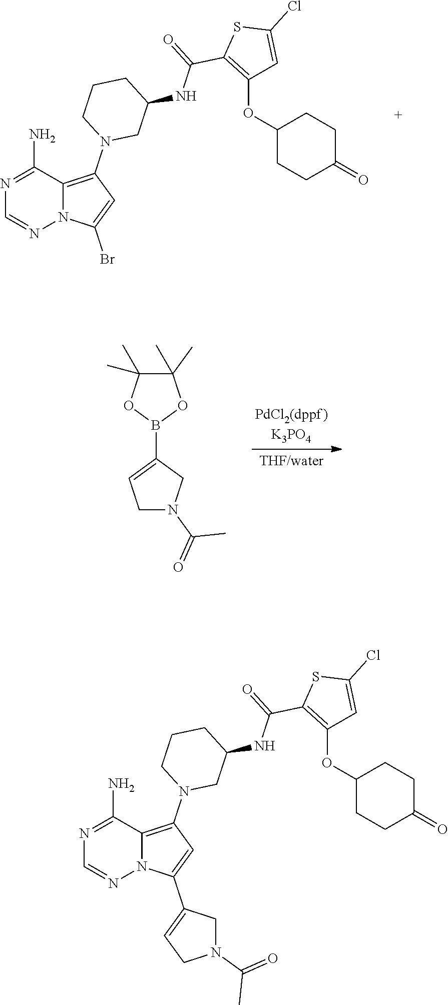 [SMILES: Nc1ncnn2c(Br)cc(N3CCC[C@@H](NC(=O)c4sc(Cl)cc4OC4CCC(=O)CC4)C3)c12 ; CC(=O)N1CC=C(B2OC(C)(C)C(C)(C)O2)C1 ; CC(=O)N1CC=C(c2cc(N3CCC[C@@H](NC(=O)c4sc(Cl)cc4OC4CCC(=O)CC4)C3)c3c(N)ncnn23)C1]

In a 2-dram vial, a mixture of Example 936A (363 mg, 0.64 mmol), Intermediate 27 (182 mg, 0.77 mmol), PdCl₂(dppf) (47 mg, 0.06 mmol), potassium phosphate, tribasic (299 mg, 1.41 mmol) in DMF-water (9:1) (5 mL) was degassed with bubbling nitrogen for 10 minutes. The vial was sealed, and the reaction was stirred at 80° C. for 2 h. The mixture was diluted with ethyl acetate and filtered. The filtrate was washed once with water, twice with 10% lithium chloride and once with brine, then dried over sodium sulfate and concentrated in vacuo. The residue was chromatographed via MPLC (silica gel column, 0% to 15% methanol/dichloromethane gradient) to Example 936B (158 mg, 41% yield). MS: [M+H]⁺=598.2.

### Example 936

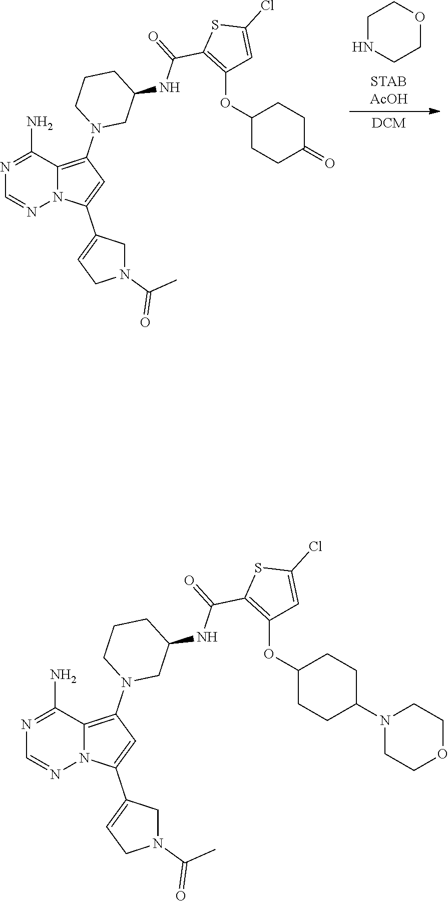 [SMILES: CC(=O)N1CC=C(c2cc(N3CCC[C@@H](NC(=O)c4sc(Cl)cc4OC4CCC(=O)CC4)C3)c3c(N)ncnn23)C1 ; C1COCCN1 ; CC(=O)N1CC=C(c2cc(N3CCC[C@@H](NC(=O)c4sc(Cl)cc4OC4CCC(N5CCOCC5)CC4)C3)c3c(N)ncnn23)C1]

A solution of Example 936B (26 mg, 0.04 mmol) and morpholine (3.8 μl, 0.04 mmol) in dichloromethane (1 mL) was treated with sodium triacetoxyborohydride (13.82 mg, 0.07 mmol) and acetic acid (2.5 ml, 0.04 mmol), and the reaction was stirred at rt for 18 h. The reaction was quenched with 1M NaOH (2 mL) and the mixture was stirred for 1 hour, then extracted 3× with dichloromethane. The combined organic phases were washed with brine, dried over sodium sulfate, and concentrated in vacuo. The mixture was diluted with DMF, filtered and purified by preparative HPLC to afford (R)—N-(1-(7-(1-Acetyl-2,5-dihydro-1H-pyrrol-3-yl)-4-aminopyrrolo[2,1-f][1,2,4]triazin-5-yl)piperidin-3-yl)-5-chloro-3-((4-morpholinocyclohexyl)oxy)thiophene-2-carboxamide, (cis/trans mixture) (16.8 mg, 0.03 mmol, 57% yield). The isomers were resolved by preparative SFC with the following conditions: Column: Chiralcel OD-H, 30 mm×250 mm, 5 μm particles; Flow Rate: 100.00 ml/min; Column Temperature: 50° C. Fractions containing the first-eluting peak were pooled and concentrated to afford Example 936 (1.7 mg, 6% yield). ¹H NMR (500 MHz, DMSO-d₆) δ 8.12-7.91 (m, 1H), 7.87 (d, J=1.9 Hz, 1H), 7.54-7.40 (m, 1H), 7.37 (s, 1H), 6.77 (br d, J=13.6 Hz, 1H), 6.72 (d, J=13.9 Hz, 1H), 4.69 (br s, 1H), 4.48 (br d, J=13.0 Hz, 2H), 4.43-4.34 (m, 1H), 4.27 (br s, 1H), 4.18-4.07 (m, 1H), 3.60-3.51 (m, 1H), 3.44-3.34 (m, 1H), 3.22-2.58 (m, 3H), 2.47-2.35 (m, 2H), 2.17-2.06 (m, 3H), 2.06-2.00 (m, 3H), 1.91-1.68 (m, 5H), 1.66-1.49 (m, 1H), 1.48-1.23 (m, 5H), 0.94-0.73 (m, 1H). LCMS Method B: RT=1.04 min, [M+H]⁺=669.2.

### Example 937

### Preparation of N—((R)-1-(4-Amino-7-(4-carbamoyl-3-fluorophenyl)pyrrolo[2,1-f][1,2,4]triazin-5-yl)piperidin-3-yl)-4-(((1s,4S)-4-(dimethylamino)cyclohexyl)oxy)-2-methylthiazole-5-carboxamide

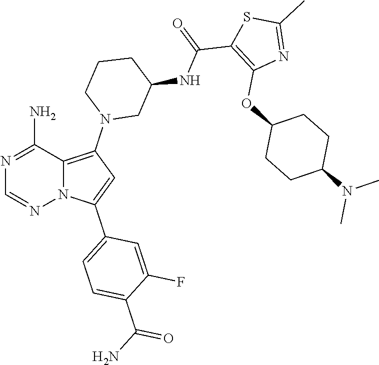 [SMILES: Cc1nc(O[C@H]2CC[C@@H](N(C)C)CC2)c(C(=O)N[C@@H]2CCCN(c3cc(-c4ccc(C(N)=O)c(F)c4)n4ncnc(N)c34)C2)s1]

### Example 937A. Ethyl 4-(((1s,4s)-4-(dimethylamino)cyclohexyl)oxy)-2-methylthiazole-5-carboxylate

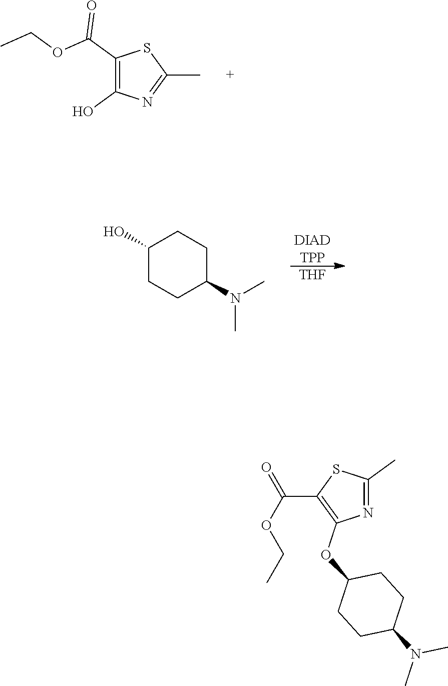

In a dried 40 mL vial, a stirring solution of triphenylphosphine (419 mg, 1.60 mmol) in anhydrous THF (5 mL) was cooled to 0° C. and treated dropwise with DIAD (0.311 mL, 1.60 mmol) at a rate of ~1 drop/5 seconds. The mixture was stirred at 0° C. for 30 minutes, during which time a colorless precipitate was observed. The mixture was treated with the slow dropwise addition of a solution of ethyl 4-hydroxy-2-methylthiazole-5-carboxylate (249 mg, 1.33 mmol) and (1r,4r)-4-(dimethylamino)cyclohexan-1-ol (229 mg, 1.6 mmol) in anhydrous THF (3 mL). The reaction was stirred for 20 minutes, at which point a homogeneous solution was observed, then allowed to come to room temperature and stirred for one hour. The mixture was concentrated in vacuo, and the residue was chromatographed via MPLC over a 40 g silica gel column, eluting at 40 mL/min with a 0% to 25% [3M ammonia in methanol]/dichloromethane gradient. Fractions containing the desired product were pooled and concentrated in vacuo to yield Example 937A (242 mg, 58% yield) as a colorless oil. MS: (m/z)=313.0 (M+H)⁺.

### Example 937B. 4-(((1s,4s)-4-(Dimethylamino)cyclohexyl)oxy)-2-methylthiazole-5-carboxylic acid

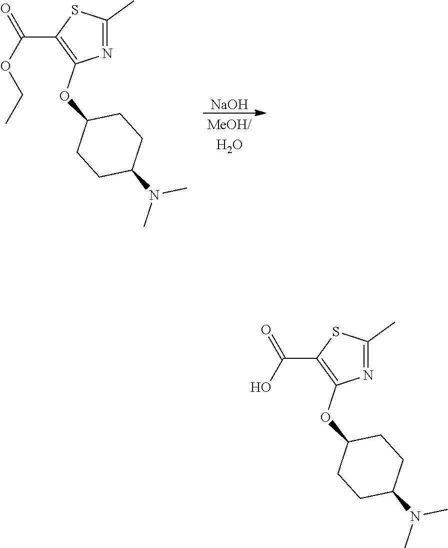 [SMILES: CCOC(=O)c1sc(C)nc1O[C@H]1CC[C@@H](N(C)C)CC1 ; "NaOH" ; Cc1nc(O[C@H]2CC[C@@H](N(C)C)CC2)c(C(=O)O)s1]

A stirring solution of Example 937A (242 mg, 0.78 mmol) in methanol (3 mL) was treated with 1M NaOH (1.2 mL, 1.2 mmol). The reaction was heated to 70° C. and stirred for 16 hours, at which point it was judged to be complete by LCMS. The mixture was diluted with water, and the methanol was evaporated. The remaining aqueous solution was washed 3× with diethyl ether, then treated with 1M HCl (1.3 mL, 1.3 mmol). The turbid solution was concentrated in vacuo to yield Example 937B. MS: (M+H)⁺=284.9.

### Example 937

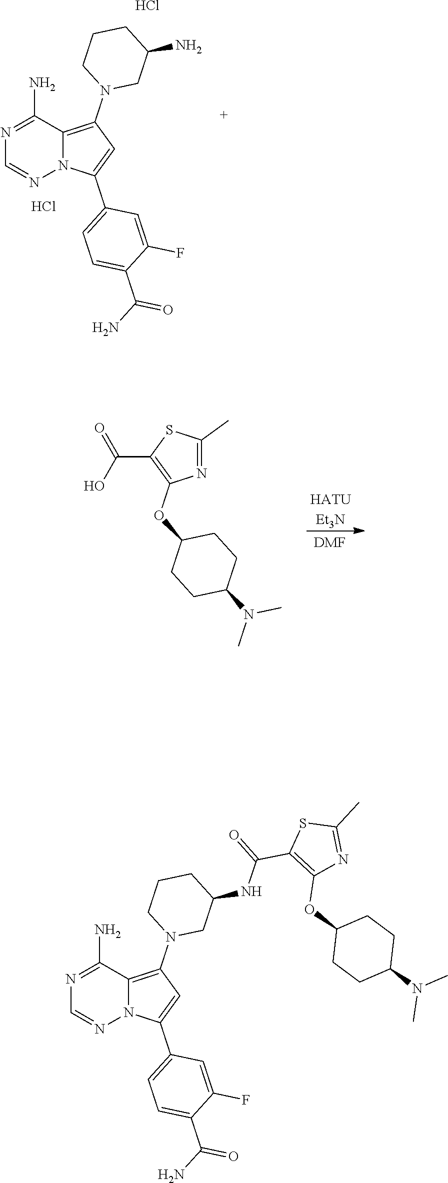

A stirring solution of Example 139D, HCl salt (19 mg, 0.05 mmol), 937C (16 mg, 0.06 mmol), and triethylamine (0.03 mL, 0.23 mmol) in DMF (2 mL) was treated with HATU (20 mg, 0.06 mmol). The reaction was stirred at rt for 3 h. The mixture was filtered and purified by preparative HPLC to provide Example 937 (10.1 mg, 34% yield); ¹H NMR (500 MHz, DMSO-d₆) δ 8.10 (br d, J=13.0 Hz, 1H), 7.97 (br d, J=8.2 Hz, 1H), 7.90 (s, 1H), 7.74 (t, J=8.2 Hz, 1H), 7.72-7.67 (m, 1H), 7.62 (br s, 1H), 7.41-7.23 (m, 1H), 7.18 (s, 1H), 5.11 (br s, 1H), 4.25-4.09 (m, 1H), 3.31-3.17 (m, 1H), 3.04-2.76 (m, 1H), 2.59 (s, 3H), 2.23-1.96 (m, 9H), 1.90 (s, 2H), 1.86-1.75 (m, 2H), 1.72-1.37 (m, 7H). LCMS Method B: RT=1.13 min, [M+H]⁺=636.1.

### Example 938

### Preparation of N—((R)-1-(4-Amino-7-(4-carbamoyl-3-fluorophenyl)pyrrolo[2,1-f][1,2,4]triazin-5-yl)piperidin-3-yl)-4-(((1s,4S)-4-(isopropylamino)cyclohexyl)oxy)-2-methylthiazole-5-carboxamide

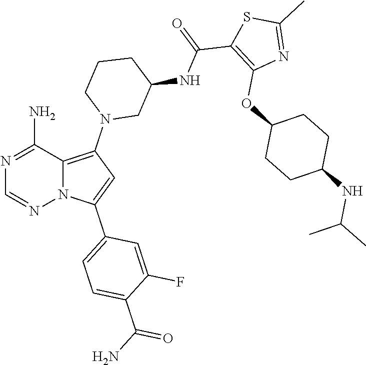 [SMILES: Cc1nc(O[C@H]2CC[C@@H](NC(C)C)CC2)c(C(=O)N[C@@H]2CCCN(c3cc(-c4ccc(C(N)=O)c(F)c4)n4ncnc(N)c34)C2)s1]

### Example 938A. Ethyl 4-(((1s,4s)-4-((tert-butoxycarbonyl)amino)cyclohexyl)oxy)-2-methylthiazole-5-carboxylate

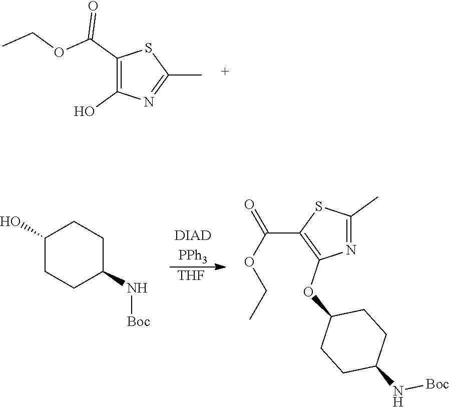 [SMILES: CCOC(=O)c1sc(C)nc1O ; CC(C)(C)OC(=O)N[C@H]1CC[C@H](O)CC1 ; CCOC(=O)c1sc(C)nc1O[C@H]1CC[C@@H](NC(=O)OC(C)(C)C)CC1 ; "DIADPPh3" ; "THF"]

In a dried 40 mL vial, a stirring solution of triphenylphosphine (697 mg, 2.66 mmol) in anhydrous THF (10 mL) was cooled to 0° C. and treated dropwise with DIAD (0.52 mL, 2.66 mmol) at a rate of ~1 drop/5 seconds. The mixture was stirred at 0° C. for 30 minutes, during which time a colorless precipitate was observed. The mixture was treated with the slow dropwise addition of a solution of ethyl 4-hydroxy-2-methylthiazole-5-carboxylate (414 mg, 2.21 mmol) and tert-butyl ((1r,4r)-4-hydroxycyclohexyl)carbamate (572 mg, 2.66 mmol) in anhydrous THF (5 mL). The reaction was stirred for 20 min, at which point a homogeneous solution was observed, then allowed to come rt and stirred for 4 d. A second portion of betaine was prepared as described above from triphenylphosphine (697 mg, 2.66 mmol) and DIAD (0.52 mL, 2.66 mmol) in anhydrous THF (5 mL). tert-butyl ((1r,4r)-4-hydroxycyclohexyl)carbamate (572 mg, 2.66 mmol) was added to the original reaction mixture, and this solution was added dropwise to the new lot of betaine at 0° C. Once the addition was complete, the mixture was allowed to slowly come to rt and stirred for 3 h. The reaction mixture was concentrated onto Celite and chromatographed via MPLC (solid loading, 0% to 100% ethyl acetate/hexanes gradient) to yield Example 938A (616 mg, 72% yield). MS: (M+H)⁺=385.0; ¹H NMR (500 MHz, CDCl₃) δ 5.25-5.09 (m, 1H), 4.58 (br d, J=5.4 Hz, 1H), 4.30 (q, J=7.1 Hz, 2H), 3.57 (br s, 1H), 2.62 (s, 3H), 2.09-1.99 (m, 2H), 1.87-1.77 (m, 21H), 1.76-1.65 (m, 4H), 1.47 (s, 9H), 1.36 (t, J=7.1 Hz, 3H).

### Example 938B. 4-(((1s,4s)-4-((tert-Butoxycarbonyl)amino)cyclohexyl)oxy)-2-methylthiazole-5-carboxylic acid

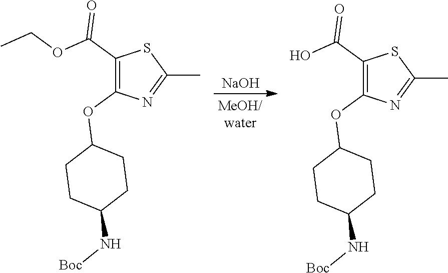

A solution of Example 938A (616 mg, 1.60 mmol) in methanol (5 mL) was treated with 1M sodium hydroxide (2.4 mL, 2.4 mmol), and the reaction was stirred at 50° C. for 6 hours, at which point it was judged to be complete by LCMS. The mixture was diluted with water (3 mL), and the methanol was evaporated. The remaining turbid solution was washed 3× with diethyl ether, then treated with 1 M HCl (2.5 mL, 2.5 mmol). The mixture was extracted 4× with ethyl acetate, then the combined organic phases were washed once with brine, dried over sodium sulfate, and concentrated in vacuo to yield Example 938B. MS: (m/z)=357.0 (M+H)⁺.

### Example 938C. N—((R)-1-(4-Amino-7-(4-carbamoyl-3-fluorophenyl)pyrrolo[2,1-f][1,2,4]triazin-5-yl)piperidin-3-yl)-4-(((1s,4S)-4-aminocyclohexyl)oxy)-2-methylthiazole-5-carboxamide dihydrochloride

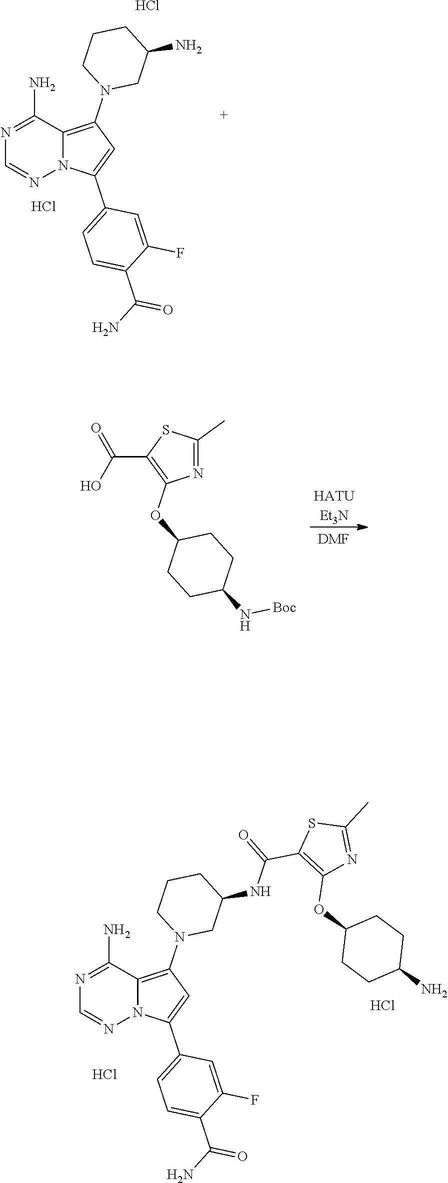

A stirring solution of Example 139D, HCl salt (135 mg, 0.33 mmol), Example 938B (142 mg, 0.40 mmol), and triethylamine (0.23 mL, 1.66 mmol) in DMF (2 mL) was treated with HATU (139 mg, 0.37 mmol). The reaction was stirred at room temperature for 3 h. The mixture was treated with water (2 mL) and stirred for 20 minutes. The resulting precipitate was collected by filtration, rinsed 3× with water and 2× with diethyl ether, and dried under vacuum to yield tert-butyl ((1s,4S)-4-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)cyclohexyl)carbamate (209 mg, 89% yield). MS: (M+H)⁺=708.5; ¹H NMR (500 MHz, DMSO-d₆) δ 8.20-8.03 (m, 2H), 8.00 (dd, J=8.3, 1.6 Hz, 1H), 7.92 (s, 1H), 7.74 (t, J=8.2 Hz, 1H), 7.63 (br d, J=11.8 Hz, 2H), 7.23 (s, 1H), 7.21-7.12 (m, 1H), 6.93-6.68 (m, 1H), 5.04 (br s, 1H), 4.20-4.07 (m, 1H), 3.38-3.32 (m, 1H), 3.12-2.99 (m, 1H), 2.94-2.68 (m, 2H), 2.60 (s, 3H), 2.08-1.80 (m, 5H), 1.77-1.44 (m, 7H), 1.43-1.23 (m, 10H). The material was taken up in dichloromethane (5 mL), and the solution was treated with 4M HCl in dioxane (5 mL). The reaction was stirred at rt for 2 h. The mixture was concentrated in vacuo, and the residue was concentrated twice from dichloromethane then dried under vacuum to yield Example 938C (191 mg, 89% yield). MS: (m/z)=608.3 (M+H)⁺.

### Example 938

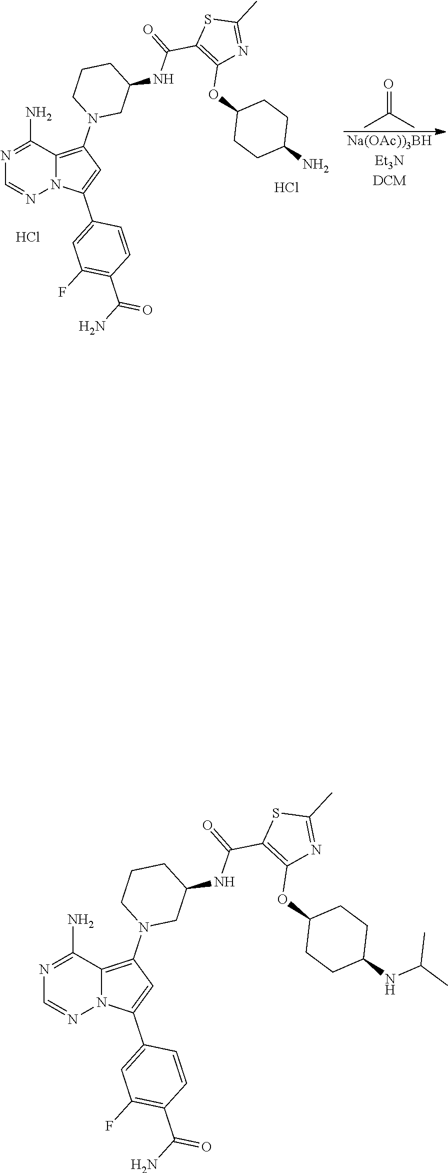

A stirring mixture Example 938C (22 mg, 0.03 mmol), triethylamine (19 μl, 0.14 mmol), and acetone (55 μl, 0.75 mmol) in dichloromethane (1 mL) was treated with sodium triacetoxyborohydride (11 mg, 0.05 mmol). The reaction was stirred at rt for 48 h. The mixture was diluted with dichloromethane (5 mL) and washed twice with 1M NaOH. The combined aqueous phases were extracted once with dichloromethane, and the combined organic phases were washed with brine, dried over sodium sulfate, and concentrated in vacuo. The residue was purified by preparative HPLC to yield Example 938 (16 mg, 71% yield); ¹H NMR (500 MHz, DMSO-d₆) δ 8.43-8.34 (m, 1H), 8.34-8.24 (m, 1H), 8.11 (br d, J=12.8 Hz, 1H), 8.00 (br d, J=7.5 Hz, 1H), 7.97 (s, 1H), 7.76 (t, J=8.1 Hz, 1H), 7.71-7.62 (m, 2H), 7.23 (s, 1H), 4.99 (br s, 1H), 4.18-4.08 (m, 1H), 3.38-3.12 (m, 1H), 3.02-2.84 (m, 2H), 2.60 (s, 3H), 2.29-2.17 (m, 2H), 2.05-1.91 (m, 1H), 1.85 (br d, J=4.2 Hz, 3H), 1.77-1.68 (m, 2H), 1.66-1.55 (m, 2H), 1.53-1.43 (m, 1H), 1.16-1.01 (m, 6H). LCMS Method B: RT=1.15 min, [M+H]⁺=650.2.

### Example 939

### Preparation of N—((R)-1-(4-Amino-7-(4-carbamoyl-3-fluorophenyl)pyrrolo[2,1-f][1,2,4]triazin-5-yl)piperidin-3-yl)-4-((cis-3-(dimethylamino)cyclohexyl)oxy)-2-methylthiazole-5-carboxamide, isomer 1

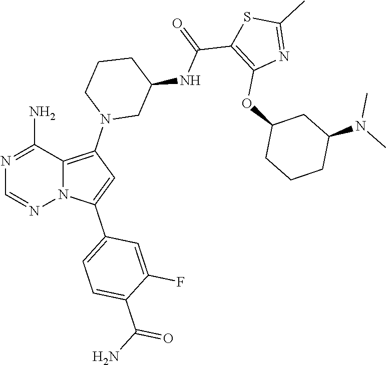 [SMILES: Cc1nc(O[C@@H]2CCC[C@H](N(C)C)C2)c(C(=O)N[C@@H]2CCCN(c3cc(-c4ccc(C(N)=O)c(F)c4)n4ncnc(N)c34)C2)s1]

### Example 939A. rel-Ethyl 4-((cis-3-((tert-butoxycarbonyl)amino)cyclohexyl)oxy)-2-methylthiazole-5-carboxylate

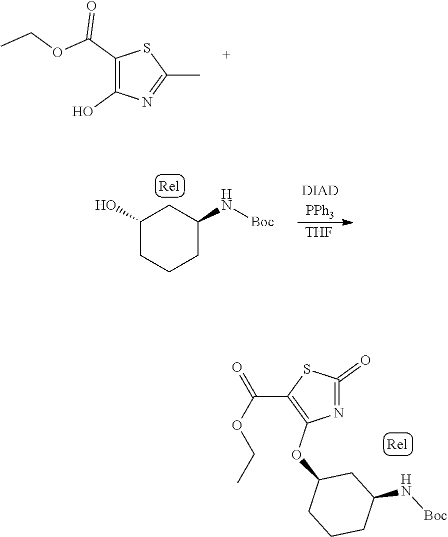

According to the preparation of Example 938A, coupling of ethyl 4-hydroxy-2-methylthiazole-5-carboxylate (267 mg, 1.42 mmol) and tert-butyl (rel-trans-3-hydroxycyclohexyl)carbamate (prepared as described in WO2012145569A1) afforded Example 939A (281 mg, 51% yield). MS: (m/z)=385.0 (M+H)⁺; ¹H NMR (500 MHz, CDCl₃) δ 5.24 (br s, 1H), 4.39-4.26 (m, 2H), 3.94 (br s, 1H), 2.63 (s, 3H), 2.09-1.98 (m, 1H), 1.95-1.84 (m, 3H), 1.81-1.67 (m, 2H), 1.46 (s, 1H), 1.36 (t, J=7.1 Hz, 3H).

### Example 939B. rel-4-((cis-3-((tert-Butoxycarbonyl)amino)cyclohexyl)oxy)-2-methylthiazole-5-carboxylic acid

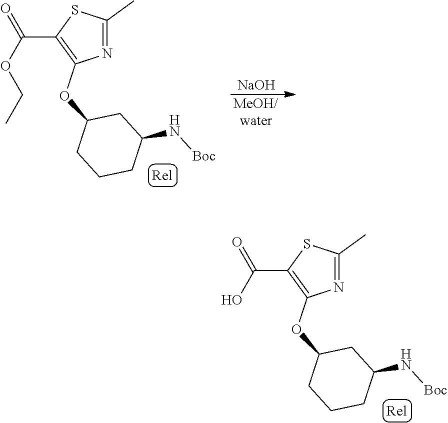 [SMILES: CCOC(=O)c1sc(C)nc1O[C@@H]1CCC[C@H](NC(=O)OC(C)(C)C)C1 ; "NaOH" ; "MeOH/water" ; Cc1nc(O[C@@H]2CCC[C@H](NC(=O)OC(C)(C)C)C2)c(C(=O)O)s1]

According to the preparation of Example 938B, hydrolysis of Example 939A (281 mg, 0.73 mmol) afforded Example 939B (222 mg, 0.62 mmol, 85% yield). MS: (m/z)=357.0 (M+H); ¹H NMR (500 MHz, CDCl₃) δ 5.20 (dt, J=7.5, 3.9 Hz, 1H), 3.87-3.73 (m, 1H), 2.67 (s, 3H), 2.37-2.19 (m, 1H), 2.03-1.77 (m, 3H), 1.47 (s, 12H).

### Example 939C. rel-N—((R)-1-(4-Amino-7-(4-carbamoyl-3-fluorophenyl)pyrrolo[2,1-f][1,2,4]triazin-5-yl)piperidin-3-yl)-4-((cis-3-aminocyclohexyl)oxy)-2-methylthiazole-5-carboxamide dihydrochloride

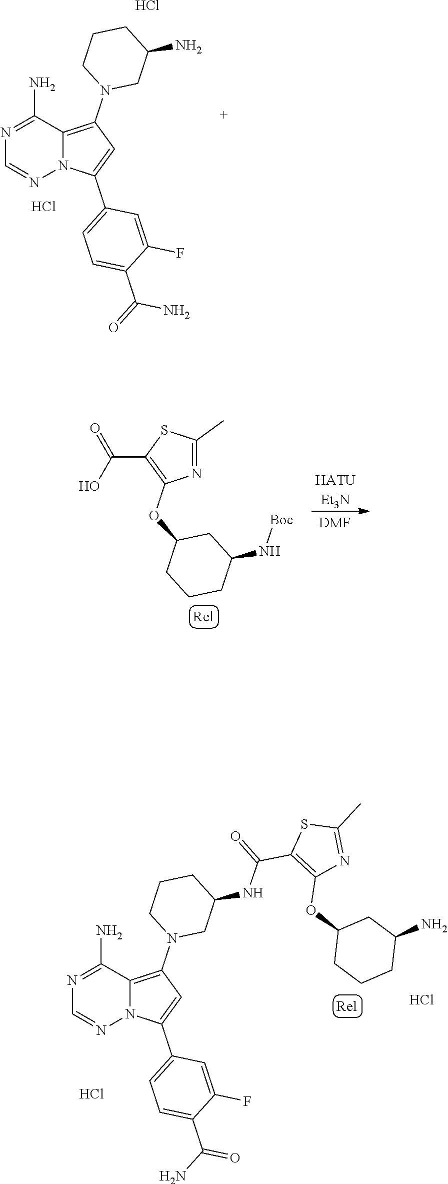

A stirring solution of Example 139D, HCl salt (80 mg, 0.20 mmol), Example 939B (70 mg, 0.20 mmol), and triethylamine (0.14 mL, 0.98 mmol) in DMF (2 mL) was treated with HATU (82 mg, 0.22 mmol). The reaction was stirred at room temperature for 16 h. The reaction mixture was concentrated in vacuo, and the residue was chromatographed via MPLC (silica gel column, 0% to 5% methanol/dichloromethane gradient), to yield rel-tert-butyl (cis-3-((5-(((S)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)cyclohexyl)carbamate (86 mg, 0.12 mmol, 62% yield); ¹H NMR (500 MHz, CDCl₃) δ 8.18 (t, J=8.4 Hz, 1H), 8.15-8.10 (m, 1H), 7.92 (s, 1H), 7.82 (d, J=8.3 Hz, 1H), 6.88 (d, J=3.1 Hz, 1H), 6.81-6.69 (m, 1H), 5.93 (br s, 1H), 5.02-4.92 (m, 1H), 4.56 (br d, J=7.4 Hz, 1H), 4.33 (br s, 1H), 3.61 (br s, 1H), 3.24 (q, J=7.3 Hz, 1H), 3.17-3.02 (m, 1H), 2.64 (s, 3H), 2.57 (br d, J=6.1 Hz, 1H), 2.40-2.16 (m, 4H), 2.04-1.97 (m, 2H), 1.95-1.80 (m, 3H), 1.51-1.37 (m, 1H), 1.36-1.29 (m, 2H), 1.10 (br d, J=10.0 Hz, 1H). MS: (m/z)=708.4 (M+H)⁺. The material was taken up in dichloromethane (2 mL), and the mixture was treated with 4M HCl in dioxane (1 mL). The reaction was stirred for 2 h. The mixture was concentrated in vacuo 3× from dichloromethane then dried under vacuum to yield Example 939C (74 mg, 0.12 mmol, 62% yield). MS: (m/z)=608.4 (M+H)⁺.

### Example 939

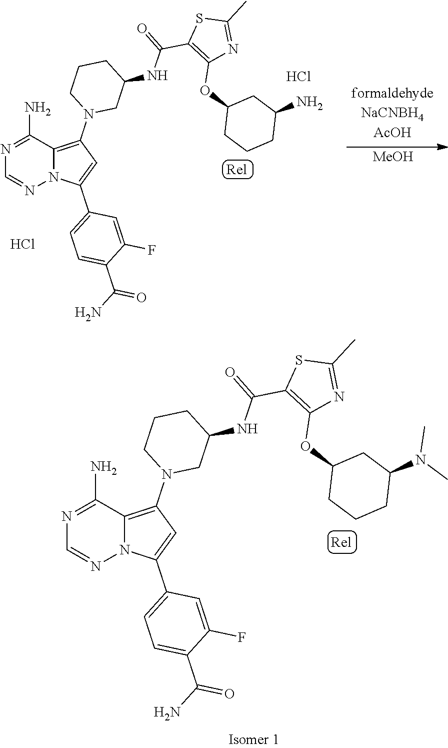

A stirring solution of Example 939C, formaldehyde (0.04 mL, 0.57 mmol), and acetic acid (0.04 mL, 0.69 mmol) in methanol (2 mL) was treated with sodium cyanoborohydride (36 mg, 0.57 mmol). The reaction was stirred at rt for 18 h. Most of the methanol was evaporated, and the remaining solution was diluted with dichloromethane (10 mL). The turbid solution was washed with 1.5 M K₂HPO₄, the layers were separated, and the aqueous phase was extracted with dichloromethane. The combined organic phases were dried over sodium sulfate and concentrated in vacuo. The residue was purified via preparative HPLC to afford rel-N—((R)-1-(4-amino-7-(4-carbamoyl-3-fluorophenyl)pyrrolo[2,1-f][1,2,4]triazin-5-yl)piperidin-3-yl)-4-((cis-3-(dimethylamino)cyclohexyl)oxy)-2-methylthiazole-5-carboxamide (69 mg, 0.11 mmol, 94% yield). The isomers were resolved via preparative SFC with the following conditions: Column: Chiralpak AS-H, 30 mm×250 mm, 5 μm particles; Flow Rate:100.00 mL/min; Column Temperature: 50° C. Fractions containing the first-eluting isomer were combined and dried via centrifugal evaporation to yield Example 939 (8.5 mg, 11% yield); ¹H NMR (500 MHz, DMSO-d₆) δ 8.19 (d, J=2.6 Hz, 1H), 8.08 (br d, J=13.0 Hz, 1H), 7.93 (br d, J=8.2 Hz, 1H), 7.85 (d, J=2.5 Hz, 1H), 7.75 (td, J=8.2, 2.4 Hz, 1H), 7.58 (br d, J=10.4 Hz, 2H), 7.49-7.31 (m, 1H), 7.15 (d, J=2.3 Hz, 1H), 4.86-4.75 (m, 1H), 4.19-4.08 (m, 1H), 3.65-3.57 (m, 2H), 3.09-2.78 (m, 2H), 2.59 (d, J=2.4 Hz, 3H), 2.43-2.33 (m, 1H), 2.29 (br d, J=11.4 Hz, 1H), 2.19-2.05 (m, 7H), 1.91-1.78 (m, 3H), 1.75-1.67 (m, 2H), 1.64-1.48 (m, 1H), 1.37-1.18 (m, 3H), 1.13-1.01 (m, 1H). LCMS Method B: RT=1.07 min, [M+H]⁺=636.1.

### Example 940

### Preparation of N—((R)-1-(4-Amino-7-(4-carbamoyl-3-fluorophenyl)pyrrolo[2,1-f][1,2,4]triazin-5-yl)piperidin-3-yl)-4-(((1S,3R)-3-(isopropylamino)cyclopentyl)oxy)-2-methylthiazole-5-carboxamide

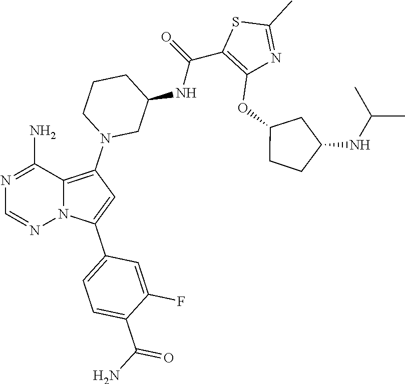 [SMILES: Cc1nc(O[C@H]2CC[C@@H](NC(C)C)C2)c(C(=O)N[C@@H]2CCCN(c3cc(-c4ccc(C(N)=O)c(F)c4)n4ncnc(N)c34)C2)s1]

### Example 940A. Ethyl 4-(((1S,3R)-3-((tert-butoxycarbonyl)amino)cyclopentyl)oxy)-2-methylthiazole-5-carboxylate

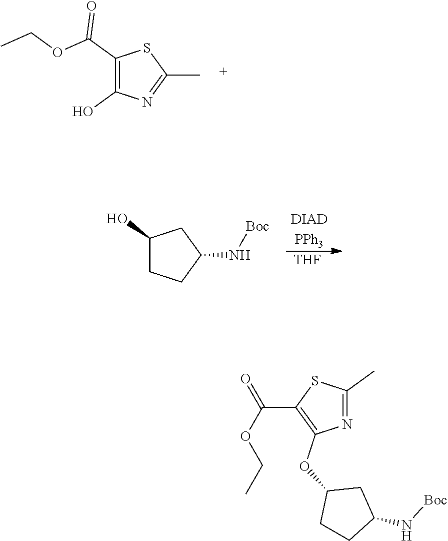 [SMILES: CCOC(=O)c1sc(C)nc1O ; CC(C)(C)OC(=O)N[C@@H]1CC[C@@H](O)C1 ; CCOC(=O)c1sc(C)nc1O[C@H]1CC[C@@H](NC(=O)OC(C)(C)C)C1 ; "DIADPPh3" ; "THF"]

According to the preparation of Example 938A, coupling of ethyl 4-hydroxy-2-methylthiazole-5-carboxylate (227 mg, 1.21 mmol) and tert-butyl ((1R,3R)-3-hydroxycyclopentyl)carbamate (244 mg, 1.21 mmol) afforded after purification Example 940A (456 mg, 100% yield). MS: (m/z)=370.9 (M+H)⁺; ¹H NMR (500 MHz, CDCl₃) δ 6.14-6.01 (m, 1H), 5.53 (br s, 1H), 4.43-4.25 (m, 3H), 2.64 (s, 3H), 2.27-1.75 (m, 6H), 1.46 (s, 9H), 1.36 (t, J=7.1 Hz, 3H).

### Example 940B. 4-(((1S,3R)-3-((tert-Butoxycarbonyl)amino)cyclopentyl)oxy)-2-methylthiazole-5-carboxylic acid

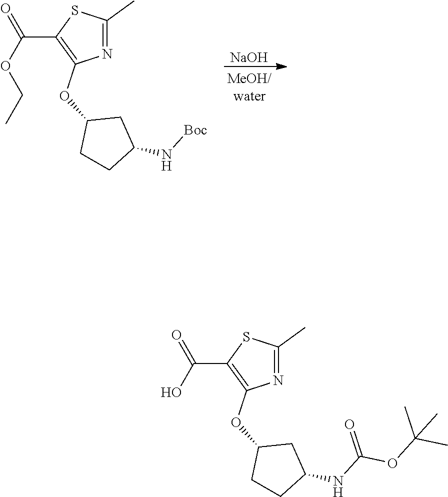 [SMILES: CCOC(=O)c1sc(C)nc1O[C@H]1CC[C@@H](NC(=O)OC(C)(C)C)C1 ; "NaOH" ; Cc1nc(O[C@H]2CC[C@@H](NC(=O)OC(C)(C)C)C2)c(C(=O)O)s1]

According to the preparation of Example 938B, hydrolysis of Example 940A (456 mg, 1.23 mmol) afforded Example 940B (312 mg, 74.0% yield). MS: (m/z)=342.9 (M+H)⁺. H NMR (400 MHz, CDCl₃) δ 5.83-5.69 (m, 1H), 5.56 (br s, 1H), 4.31 (br s, 1H), 2.67 (s, 3H), 2.25-2.03 (m, 3H), 2.00-1.86 (m, 2H), 1.80 (ddd, J=12.7, 8.5, 4.1 Hz, 1H), 1.47 (s, 9H).

### Example 940C. N—((R)-1-(4-Amino-7-(4-carbamoyl-3-fluorophenyl)pyrrolo[2,1-f][1,2,4]triazin-5-yl)piperidin-3-yl)-4-(((1S,3R)-3-aminocyclopentyl)oxy)-2-methylthiazole-5-carboxamide dihydrochloride

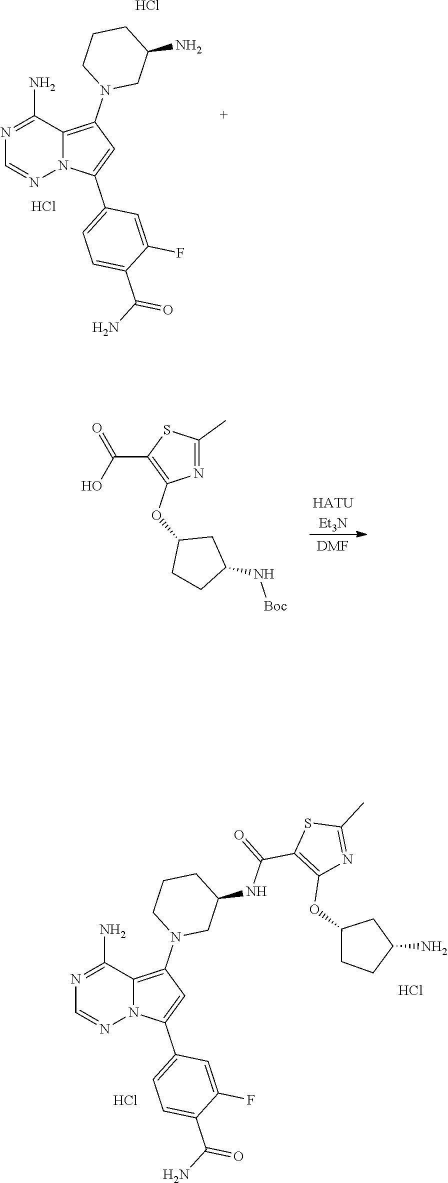

According to the procedure for the preparation of Example 939C, coupling of Example 139D, HCl salt (84 mg, 0.21 mmol) with Example 940B (71 mg, 0.21 mmol), followed by treatment with 4M HCl afforded Example 940C (72 mg, 55% yield). MS: (m/z)=594.3 (M+H)⁺.

### Example 940

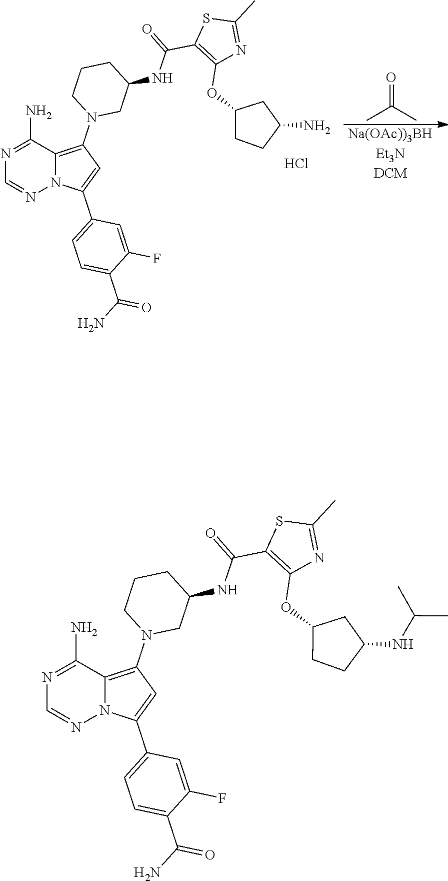

A stirring mixture of Example 940C (24 mg, 0.04 mmol), triethylamine (0.03 mL, 0.19 mmol), and acetone (0.03 mL, 0.38 mmol) in dichloromethane (1 mL) was treated with sodium triacetoxyborohydride (14 mg, 0.07 mmol). The reaction was stirred at rt for 48 h. The mixture was diluted with dichloromethane (5 mL), and the solution was washed twice with 1M NaOH and once with brine, then dried over sodium sulfate and concentrated in vacuo. The residue was purified by preparative HPLC to afford Example 940 (15.5 mg, 64% yield); ¹H NMR (500 MHz, DMSO-d₆) δ 8.22 (s, 1H), 8.10 (br d, J=12.8 Hz, 1H), 7.95 (br d, J=7.8 Hz, 1H), 7.87 (s, 1H), 7.75 (t, J=8.2 Hz, 1H), 7.59 (br d, J=6.6 Hz, 2H), 7.44-7.28 (m, 1H), 7.17 (s, 1H), 5.29 (br d, J=1.6 Hz, 1H), 4.21-4.08 (m, 1H), 3.59-3.43 (m, 1H), 3.26-3.18 (m, 1H), 3.06-2.92 (m, 1H), 2.81-2.71 (m, 1H), 2.59 (s, 3H), 2.47-2.34 (m, 2H), 1.97-1.88 (m, 4H), 1.87-1.80 (m, 7H), 1.64-1.48 (m, 3H), 0.96 (br t, J=7.2 Hz, 6H). LCMS Method B: RT=1.22 min, [M+H]⁺=636.2.

### Example 941

### Preparation of (R)—N-(1-(4-amino-7-(4-carbamoyl-3-fluorophenyl)pyrrolo[2,1-f][1,2,4]triazin-5-yl)piperidin-3-yl)-4-((4-(cyclopropylamino)cyclohexyl)oxy)-2-methylthiazole-5-carboxamide, isomer 2

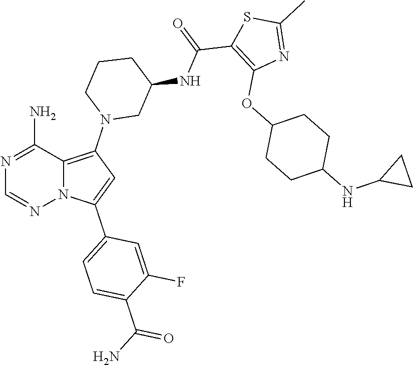 [SMILES: Cc1nc(OC2CCC(NC3CC3)CC2)c(C(=O)N[C@@H]2CCCN(c3cc(-c4ccc(C(N)=O)c(F)c4)n4ncnc(N)c34)C2)s1]

### Example 941A. Ethyl 4-((4-hydroxycyclohexyl)oxy)-2-methylthiazole-5-carboxylate

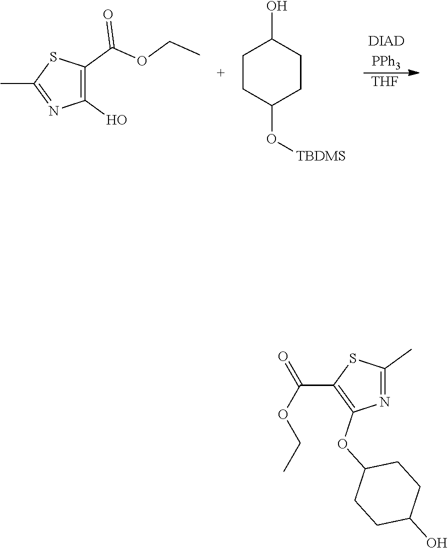

A stirred solution of triphenylphosphine (3.23 g, 12.30 mmol) in anhydrous THF (60 mL) at 0° C. was treated dropwise with DIAD (2.4 mL, 12.30 mmol) at a rate of ~1 drop/5 seconds. The mixture was stirred at 0° C. for 30 minutes, during which time a colorless precipitate was observed. The mixture was treated with the slow dropwise addition of a solution of ethyl 4-hydroxy-2-methylthiazole-5-carboxylate (0.677 g, 3.62 mmol) and 4-((tert-butyldimethylsilyl)oxy)cyclohexan-1-ol (1 g, 4.34 mmol) in anhydrous THF (15 mL). The reaction was stirred for 20 min, then allowed to come to rt and stirred for 2 d. The reaction mixture was concentrated in vacuo. The residue was chromatographed via MPLC (silica gel column, 0% to 25% ethyl acetate/hexanes gradient) to yield ethyl 4-((4-((tert-butyldimethylsilyl)oxy)cyclohexyl)oxy)-2-methylthiazole-5-carboxylate (2.5 g, 86% yield). MS: (m/z)=400.2 (M+H)⁺. The material was dissolved in THF (10 mL), and the solution was treated with TBAF (1M in THF) (21.7 mL, 21.7 mmol). The reaction was stirred at room temperature for 18 h. The mixture was diluted with ethyl acetate (100 mL), and the turbid solution was washed 3× with water and once with brine, then dried over sodium sulfate and concentrated in vacuo. The residue was chromatographed via MPLC (25% to 60% ethyl acetate/hexanes gradient) to yield Example 941A (1.13 g, 55% Yield). MS: (m/z)=286.1 (M+H)⁺.

### Example 941B. Ethyl 2-methyl-4-((4-oxocyclohexyl)oxy)thiazole-5-carboxylate

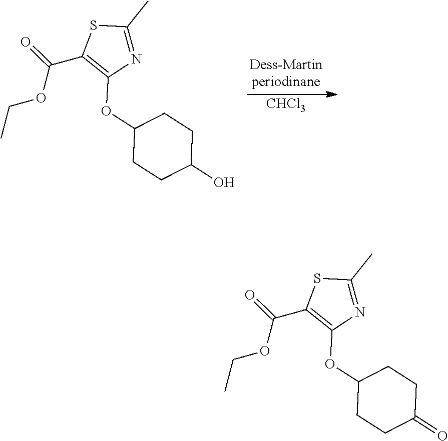 [SMILES: CCOC(=O)c1sc(C)nc1OC1CCC(O)CC1 ; "CHCl3" ; CCOC(=O)c1sc(C)nc1OC1CCC(=O)CC1]

A stirred solution of Example 941A (1.13 g, 3.96 mmol) in chloroform (5 mL) at 0° C., was treated with Dess-Martin periodinane (1.85 g, 4.36 mmol). The reaction was allowed to slowly come to rt and stir for 18 h. The mixture was treated with additional Dess-Martin periodinane (0.6 g, 1.42 mmol), and the reaction was stirred for 7 h. The mixture was treated with half saturated sodium thiosulfate and half saturated sodium bicarbonate and stirred for 30 min. Solids were removed by filtration, the layers were separated, and the aqueous phase was extracted twice with dichloromethane. The combined organic phases were washed with brine, dried over sodium sulfate, and concentrated in vacuo. The residue was chromatographed via MPLC (15% to 100% ethyl acetate/hexanes gradient) to yield Example 941B (900 mg, 80% yield). MS: (m/z)=284.0 (M+H)⁺; ¹H NMR (500 MHz, CDCl₃) δ 5.45-5.38 (m, 1H), 4.29 (q, J=7.1 Hz, 2H), 2.89-2.75 (m, 2H), 2.65 (s, 3H), 2.43-2.30 (m, 4H), 2.15-2.02 (m, 2H), 1.33 (t, J=7.1 Hz, 3H).

### Example 941C. 2-Methyl-4-((4-oxocyclohexyl)oxy)thiazole-5-carboxylic acid

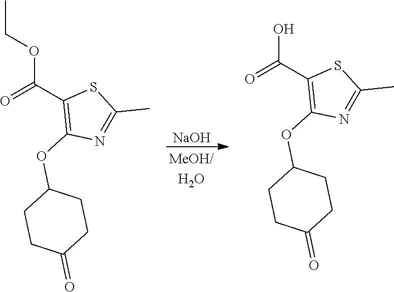

A stirring solution of Example 941B (495 mg, 1.75 mmol) in methanol (5 mL) was treated with 1M NaOH (2.6 mL, 2.6 mmol). The reaction was heated to 70° C. and stirred for 2 hours. The mixture was diluted with water and the methanol was evaporated. The aqueous mixture was washed twice with ethyl acetate, then treated with HCl (2.7 mL, 2.7 mmol). The turbid solution was extracted 3× with ethyl acetate, then the combined organic phases were washed once with brine, dried over sodium sulfate, and concentrated to yield Example 941C (0.43 g, 96% yield). MS: (m/z)=256.0 (M+H)⁺.

### Example 941D. (R)—N-(1-(4-Amino-7-(4-carbamoyl-3-fluorophenyl)pyrrolo[2,1-f][1,2,4]triazin-5-yl)piperidin-3-yl)-2-methyl-4-((4-oxocyclohexyl)oxy)thiazole-5-carboxamide

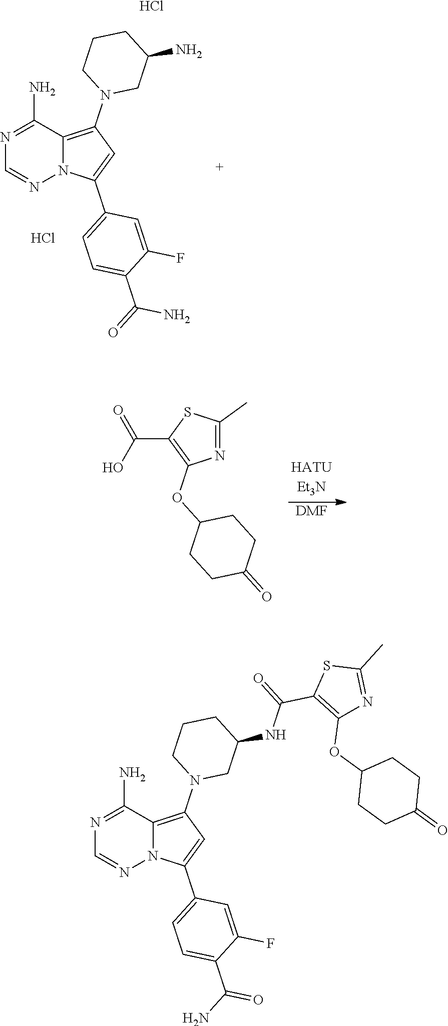

A stirring solution of Example 139D, HCl salt (278 mg, 0.69 mmol), Example 941C (210 mg, 0.82 mmol), and triethylamine (0.48 mL, 3.42 mmol) in DMF (2 mL) was treated with HATU (286 mg, 0.75 mmol). The reaction was stirred at rt for 3 h. The mixture was diluted with ethyl acetate (20 mL), and the turbid solution was washed once with water, twice with 10% lithium chloride, and once with brine. The organic phase was dried over sodium sulfate and concentrated. The residue was chromatographed via MPLC (0% to 10% methanol/dichloromethane gradient) to yield Example 941D (283 mg, 68.1% yield); ¹H NMR (500 MHz, CDCl₃) δ 8.06-8.00 (m, 2H), 7.82-7.73 (m, 2H), 6.81 (s, 1H), 5.45-5.34 (m, 1H), 4.30-4.10 (m, 1H), 3.33-3.31 (m, 2H), 3.14-3.03 (m, 1H), 2.59 (s, 3H), 2.52-2.39 (m, 4H), 2.27 (br dd, J=8.5, 4.3 Hz, 2H), 2.22-2.12 (m, 2H), 1.90-1.77 (m, 3H). LC-MS: (Waters Acquity BEH C18 1.7 mM, 2.1×50 mm; A: 95% water/5% acetonitrile+0.05% TFA; B: 95% acetonitrile/5% water+0.05% TFA; wavelength 220 nm; flow rate 1 mL/min; 1 min gradient, 0.5 min hold; 0 to 100% B) rt=0.75 min, (m/z)=607.3 (M+H)⁺.

### Example 941

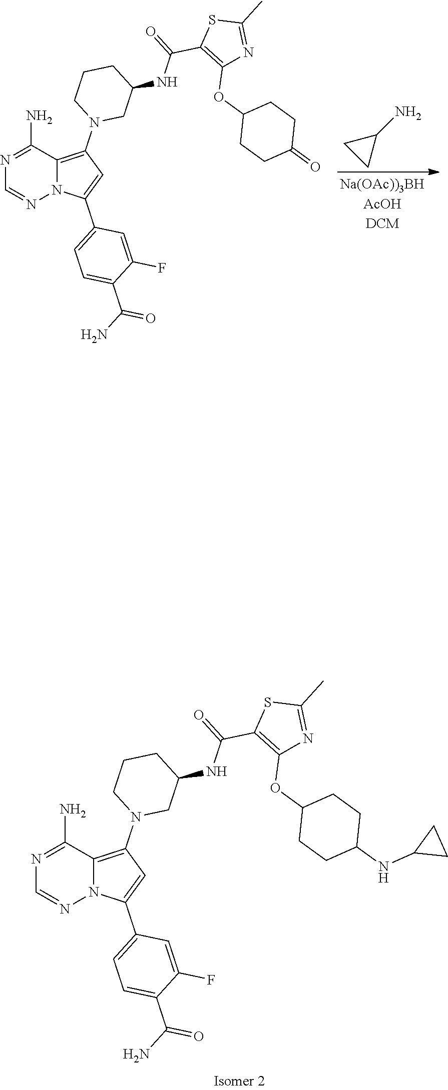

A stirring mixture of Example 941D (25 mg, 0.04 mmol), cyclopropanamine (6.3 μl, 0.09 mmol), and acetic acid (7.1 μl, 0.12 mmol) in dichloromethane (1 mL) was treated with sodium triacetoxyborohydride (26 mg, 0.12 mmol). The reaction was stirred at rt for 18 h. The solvent was evaporated, and the residue was purified by preparative HPLC to yield (R)—N-(1-(4-amino-7-(4-carbamoyl-3-fluorophenyl)pyrrolo[2,1-f][1,2,4]triazin-5-yl)piperidin-3-yl)-4-((4-(cyclopropylamino)cyclohexyl)oxy)-2-methylthiazole-5-carboxamide, cis,trans-mixture (30 mg). LCMS Method B: RT=1.12 min, [M+H]⁺=648.1. The isomers were resolved via preparative SFC (Chiralpak AS-H). Fractions containing the second-eluting isomer were combined and dried via centrifugal evaporation to yield Example 941 (4.1 mg, 14% yield); ¹H NMR (500 MHz, DMSO-d₆) δ 8.10-8.00 (m, 1H), 7.93 (br d, J=8.2 Hz, 1H), 7.85 (s, 1H), 7.69 (t, J=8.2 Hz, 1H), 7.62 (br d, J=1.1 Hz, 1H), 7.57 (br s, 1H), 7.34-7.20 (m, 1H), 7.14 (s, 1H), 5.07 (br s, 1H), 4.20-4.03 (m, 1H), 3.00-2.68 (m, 1H), 2.54 (s, 3H), 2.44-2.39 (m, 1H), 1.94-1.82 (m, 3H), 1.81-1.71 (m, 2H), 1.70-1.53 (m, 4H), 1.38-1.24 (m, 2H), 0.22-0.11 (m, 2H), 0.01 (br s, 2H). LCMS Method B: RT=1.19 min, [M+H]⁺=647.9.

### Example 942

### Preparation of (R)—N-(1-(4-Amino-7-(4-carbamoyl-3-fluorophenyl)pyrrolo[2,1-f][1,2,4]triazin-5-yl)piperidin-3-yl)-4-((5-(isopropylamino)cyclooctyl)oxy)-2-methylthiazole-5-carboxamide

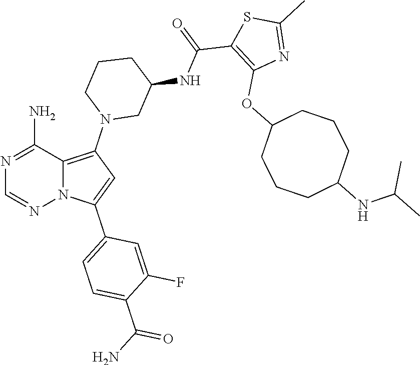 [SMILES: Cc1nc(OC2CCCC(NC(C)C)CCC2)c(C(=O)N[C@@H]2CCCN(c3cc(-c4ccc(C(N)=O)c(F)c4)n4ncnc(N)c34)C2)s1]

### Example 942A. tert-Butyl (5-hydroxycyclooctyl)carbamate

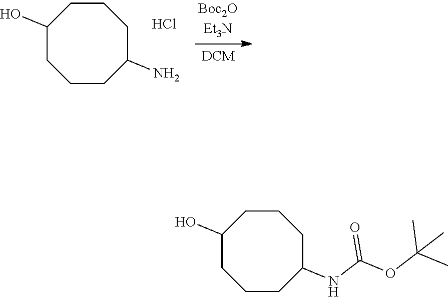 [SMILES: NC1CCCC(O)CCC1 ; Cl ; CC(C)(C)OC(=O)NC1CCCC(O)CCC1]

A suspension of 5-aminocyclooctan-1-ol hydrochloride (250 mg, 1.39 mmol) and triethylamine (0.4 mL, 2.80 mmol) in dichloromethane (5 mL) was treated dropwise with Boc-anhydride (0.388 mL, 1.67 mmol). The mixture was stirred at it for 19 h. The mixture was diluted with ethyl acetate (20 mL), and the turbid solution was washed twice with water and once with brine. The organic phase was dried over sodium sulfate and concentrated in vacuo. The residue was chromatographed via MPLC (0% to 100% ethyl acetate/hexanes gradient, ELSD detection) to yield Example 942A (340 mg, 100% yield) as a colorless solid; ¹H NMR (500 MHz, CDCl₃) δ 4.59-4.37 (m, 1H), 3.97-3.78 (m, 1H), 3.72-3.51 (m, 1H), 1.96-1.81 (m, 5H), 1.78-1.64 (m, 3H), 1.61-1.43 (m, 13H), 1.38-1.29 (m, 1H).

### Example 942B. Ethyl 4-((5-((tert-butoxycarbonyl)amino)cyclooctyl)oxy)-2-methylthiazole-5-carboxylate

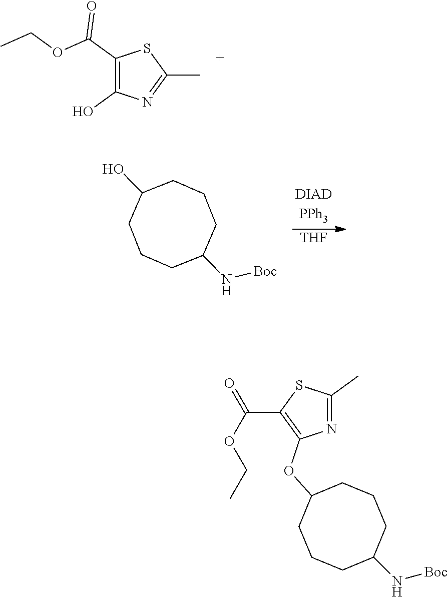 [SMILES: CCOC(=O)c1sc(C)nc1O ; CC(C)(C)OC(=O)NC1CCCC(O)CCC1 ; CCOC(=O)c1sc(C)nc1OC1CCCC(NC(=O)OC(C)(C)C)CCC1 ; "THF"]

According to the preparation of Example 938A, coupling of ethyl 4-hydroxy-2-methylthiazole-5-carboxylate (227 mg, 1.21 mmol) and Example 942A (340 mg, 1.40 mmol) afforded after purification Example 942B (541 mg, 113% yield), which was used as is without further purification. MS: (M+H)⁺=413.0; ¹H NMR (500 MHz, CDCl₃) δ 5.07 (dt, J=12.6, 6.2 Hz, 2H), 4.68-4.43 (m, 1H), 4.28 (q, J=7.1 Hz, 2H), 3.69 (br s, 1H), 2.62 (d, J=1.9 Hz, 3H), 2.04-1.87 (m, 7H), 1.86-1.76 (m, 1H), 1.75-1.62 (m, 2H), 1.61-1.52 (m, 2H), 1.46 (s, 9H), 1.35 (br d, J=1.9 Hz, 3H).

### Example 942C. 4-((5-((tert-Butoxycarbonyl)amino)cyclooctyl)oxy)-2-methylthiazole-5-carboxylic acid

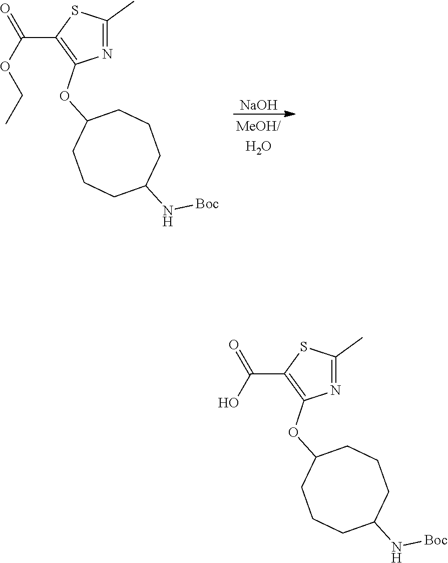 [SMILES: CCOC(=O)c1sc(C)nc1OC1CCCC(NC(=O)OC(C)(C)C)CCC1 ; "NaOH" ; Cc1nc(OC2CCCC(NC(=O)OC(C)(C)C)CCC2)c(C(=O)O)s1]

A stirring solution of Example 942B (540 mg, 1.31 mmol) in methanol (5 mL) was treated with 1M NaOH (1.9 mL, 1.9 mmol). The reaction was heated to 60° C. and stirred for 2 hours, 14 h at rt, then 60° C. for 3 hours. The mixture was diluted with water and the methanol was evaporated. The aqueous mixture was washed twice with ethyl acetate, then treated with 1M HCl (2.0 mL, 2.0 mmol). The turbid solution was extracted 3× with ethyl acetate, then the combined organic phases were washed once with brine, dried over sodium sulfate, and concentrated to yield Example 942C (280 mg, 0.73 mmol, 56% yield); ¹H NMR (500 MHz, CDCl₃) δ 5.31-5.13 (m, 1H), 4.63-4.40 (m, 1H), 3.81-3.58 (m, 1H), 2.71-2.63 (m, 3H), 2.16-2.05 (m, 3H), 2.02-1.87 (m, 6H), 1.85-1.63 (m, 4H), 1.62-1.51 (m, 3H), 1.51-1.43 (m, 9H). MS: (m/z)=385.0 (M+H)⁺.

### Example 942D. (R)—N-(1-(4-Amino-7-(4-carbamoyl-3-fluorophenyl)pyrrolo[2,1-f][1,2,4]triazin-5-yl)piperidin-3-yl)-4-((5-aminocyclooctyl)oxy)-2-methylthiazole-5-carboxamide dihydrochloride

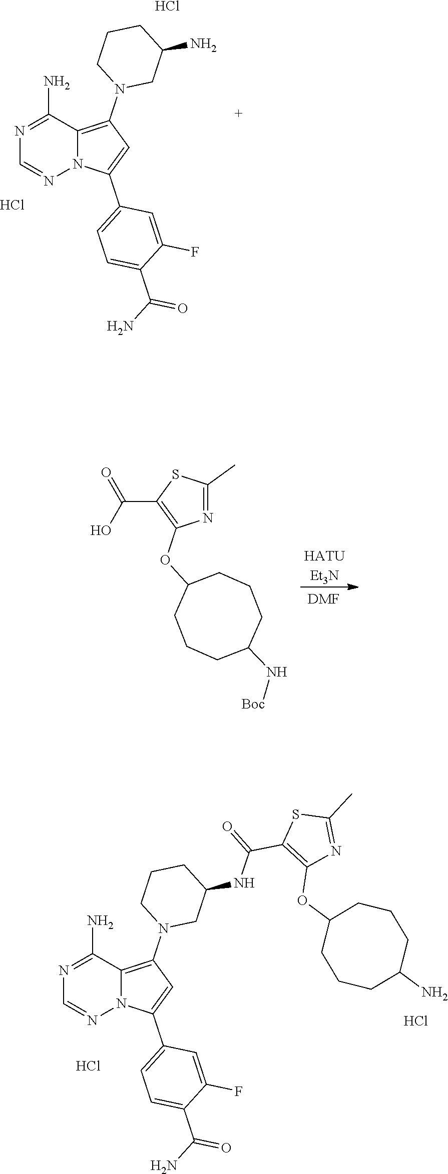

According to the procedure for the preparation of Example 939C, coupling of Example 139D, HCl salt (90 mg, 0.20 mmol) and Example 942C (86 mg, 0.22 mmol), followed by treatment with 4M HCl (104 mg, 0.14 mmol) to yield Example 942D (100 mg, 70% yield for two steps). MS: (m/z)=636.4 (M+H)⁺.

### Example 942

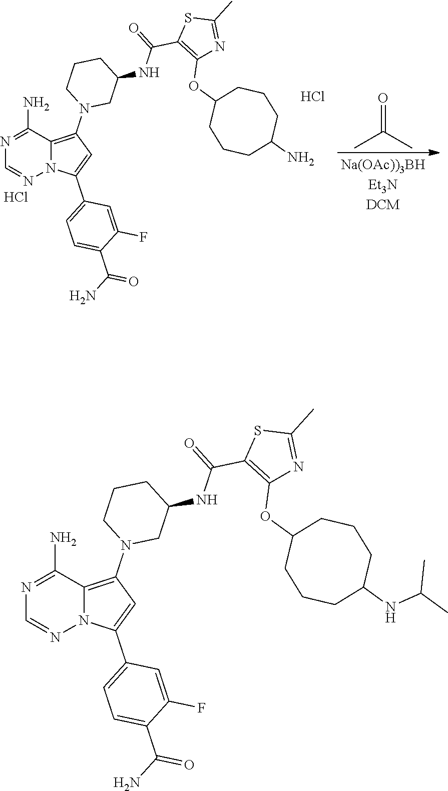

A mixture of Example 942D (50 mg, 0.07 mmol), triethylamine (0.05 mL, 0.35 mmol), and acetone (0.05 mL, 0.70 mmol) in dichloromethane (1 mL) was treated with sodium triacetoxyborohydride (22 mg, 0.11 mmol). The reaction was stirred at room temperature for 18 h. The solvent was evaporated, and the crude material purified by preparative HPLC to afford Example 942 (28.3 mg, 53% yield); ¹H NMR (500 MHz, DMSO-d₆) δ 8.16-8.04 (m, 1H), 7.98 (br d, J=8.1 Hz, 1H), 7.90 (s, 1H), 7.74 (br t, J=8.2 Hz, 1H), 7.68 (br s, 1H), 7.65-7.58 (m, 1H), 7.44-7.29 (m, 1H), 7.22 (s, 1H), 5.12-4.99 (m, 1H), 4.14 (br dd, J=7.6, 5.3 Hz, 1H), 3.67-3.51 (m, 1H), 3.22-3.11 (m, 1H), 2.98-2.89 (m, 2H), 2.83-2.72 (m, 1H), 2.59 (s, 3H), 2.04-1.68 (m, 13H), 1.67-1.29 (m, 6H), 1.08-0.87 (m, 6H). LCMS Method B: RT=1.4 min, [M+H]⁺=678.3.

### Example 943

### Preparation of (R)—N-(1-(4-Amino-7-(4-carbamoyl-3-fluorophenyl)pyrrolo[2,1-f][1,2,4]triazin-5-yl)piperidin-3-yl)-4-((4-(azetidin-1-yl)cyclohexyl)oxy)-2-methylthiazole-5-carboxamide

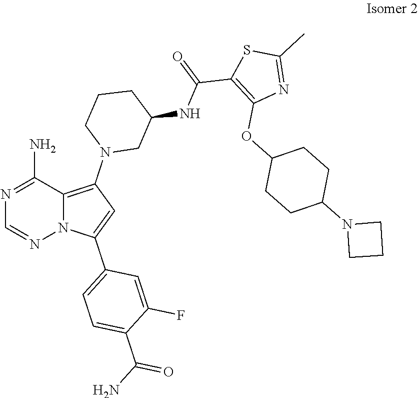 [SMILES: Cc1nc(OC2CCC(N3CCC3)CC2)c(C(=O)N[C@@H]2CCCN(c3cc(-c4ccc(C(N)=O)c(F)c4)n4ncnc(N)c34)C2)s1]

Example 943 was prepared from Example 941D (52 mg, 0.09 mmol) and azetidine hydrochloride (24 mg, 0.26 mmol) using the procedure described in the final step of Example 941. The isomers were resolved via preparative SFC (Chiralpak AS-H). Fractions containing the second-eluting isomer was evaporated to yield Example 943 (11.1 mg, 20% yield); ¹H NMR (500 MHz, DMSO-d₆) δ 8.11 (br d, J=13.1 Hz, 1H), 7.98 (d, J=8.4 Hz, 1H), 7.92 (s, 1H), 7.74 (t, J=8.1 Hz, 1H), 7.68-7.59 (m, 2H), 7.49-7.28 (m, 1H), 7.23 (s, 1H), 6.99-6.58 (m, 1H), 5.04 (br s, 1H), 4.31-4.14 (m, 1H), 3.08-2.89 (m, 4H), 2.59 (s, 3H), 2.02 (br d, J=3.7 Hz, 1H), 1.94-1.74 (m, 7H), 1.71-1.55 (m, 3H), 1.52-1.38 (m, 2H), 1.34-1.23 (m, 2H). LCMS Method B: RT=1.17 min, [M+H]⁺=648.4.

### Example 944

### Preparation of (R)—N-(1-(4-Amino-7-(4-carbamoyl-3-fluorophenyl)pyrrolo[2,1-f][1,2,4]triazin-5-yl)piperidin-3-yl)-5-chloro-3-((6-(dimethylamino)spiro[3.3]heptan-2-yl)oxy)thiophene-2-carboxamide

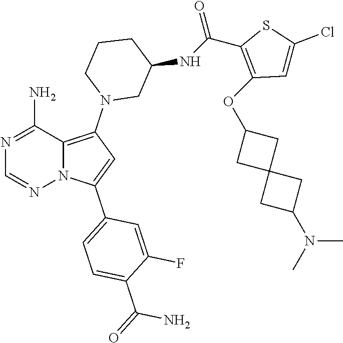 [SMILES: CN(C)C1CC2(CC(Oc3cc(Cl)sc3C(=O)N[C@@H]3CCCN(c4cc(-c5ccc(C(N)=O)c(F)c5)n5ncnc(N)c45)C3)C2)C1]

### Example 944A. Methyl 3-((6-((tert-butoxycarbonyl)amino)spiro[3.3]heptan-2-yl)oxy)-5-chlorothiophene-2-carboxylate

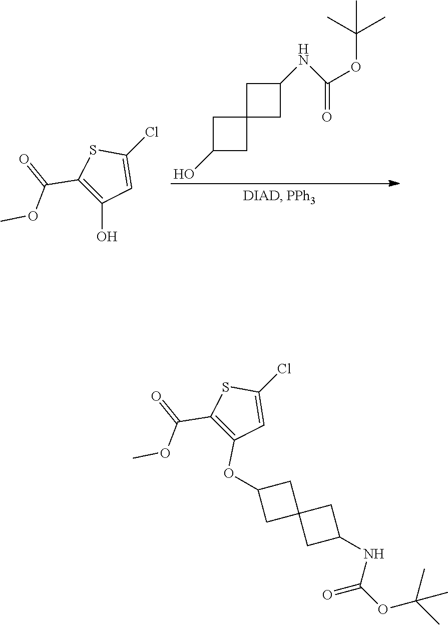 [SMILES: COC(=O)c1sc(Cl)cc1O ; CC(C)(C)OC(=O)NC1CC2(CC(O)C2)C1 ; COC(=O)c1sc(Cl)cc1OC1CC2(CC(NC(=O)OC(C)(C)C)C2)C1]

To a solution of methyl 5-chloro-3-hydroxythiophene-2-carboxylate (0.512 g, 2.66 mmol) in THF (20 mL), were added tert-butyl (6-hydroxyspiro[3.3]heptan-2-yl)carbamate (0.665 g, 2.92 mmol), triphenylphosphine (1.05 g, 3.99 mmol), and DIAD (0.775 mL, 3.99 mmol). The mixture was stirred at rt overnight. The reaction was evaporated. The compound was purified by flash chromatography (4:1 hexanes/EtOAc) to yield 1.46 g (>100%) of Example 944A as a clear orange oil, which was used without further purification. MS: (M+Na)⁺=424.2.

### Example 944B. 3-((6-((tert-Butoxycarbonyl)amino)spiro[3.3]heptan-2-yl)oxy)-5-chlorothiophene-2-carboxylic acid

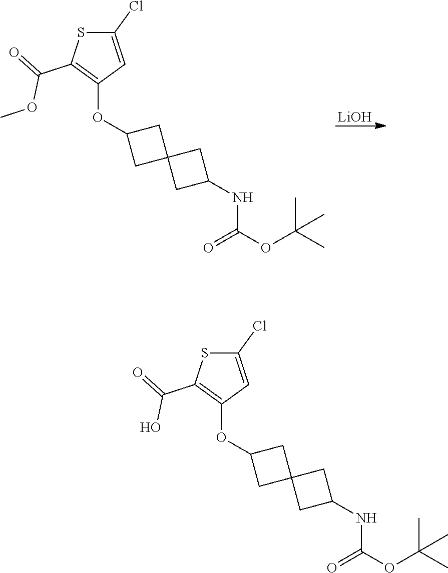 [SMILES: COC(=O)c1sc(Cl)cc1OC1CC2(CC(NC(=O)OC(C)(C)C)C2)C1 ; "LiOH" ; CC(C)(C)OC(=O)NC1CC2(C1)CC(Oc1cc(Cl)sc1C(=O)O)C2]

Example 944A (entire amount from previous step, 2.66 mmol) was dissolved in 5 mL MeOH and to it was added 2N lithium hydroxide (3.32 mL, 6.65 mmol). The mixture was stirred at rt overnight. The pH was adjusted to 4 with 1N HCl and the solvent was evaporated. EtOAc was added. The layers were separated, and the EtOAc layer washed with brine, dried (sodium sulfate) and evaporated to yield 940 mg of Example 944B as a white solid. MS: (M+Na)⁺=410.0

### Example 944C. (R)-(6-((2-((1-(4-Amino-7-(4-carbamoyl-3-fluorophenyl)pyrrolo[2,1-f][1,2,4]triazin-5-yl)piperidin-3-yl)carbamoyl)-5-chlorothiophen-3-yl)oxy)spiro[3.3]heptan-2-yl)carbamate, 2HCl salt

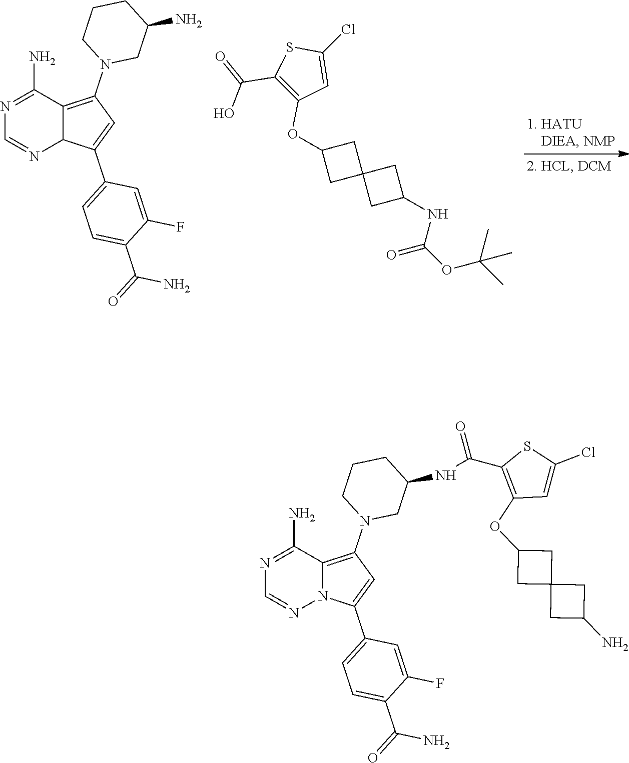 [SMILES: NC(=O)c1ccc(C2=CC(N3CCC[C@@H](N)C3)=C3C(N)=NC=NC23)cc1F ; CC(C)(C)OC(=O)NC1CC2(C1)CC(Oc1cc(Cl)sc1C(=O)O)C2 ; NC(=O)c1ccc(-c2cc(N3CCC[C@@H](NC(=O)c4sc(Cl)cc4OC4CC5(CC(N)C5)C4)C3)c3c(N)ncnn23)cc1F]

Example 139D, HCl salt (200 mg, 0.453 mmol) was dissolved in NMP (1 mL) and to this mixture was added DIEA (0.396 mL, 2.27 mmol), Example 944B (176 mg, 0.453 mmol), and HATU (190 mg, 0.498 mmol). The mixture was stirred overnight at rt. The reaction mixture was worked up by adding EtOAc and washing with 10% LiCl (3×), brine (1×), drying (sodium sulfate) and evaporating. The crude product was purified by flash chromatography (20-100% EtOAc/hexanes gradient) to yield 128.8 mg of the Boc-protected intermediate as a yellow orange solid. MS: (M+H)⁺=739.4. The intermediate was dissolved in DCM (2 mL) and to it was added 4N HCl in dioxane (1.13 mL, 4.53 mmol) and a minimum amount of MeOH to dissolve the formed precipitate. The mixture was stirred at rt overnight. The reaction was evaporated and then re-evaporated three times from to yield 169.1 mg of Example 944C as a yellow brown gummy solid. MS: (M+H)⁺=639.4.

### Example 944

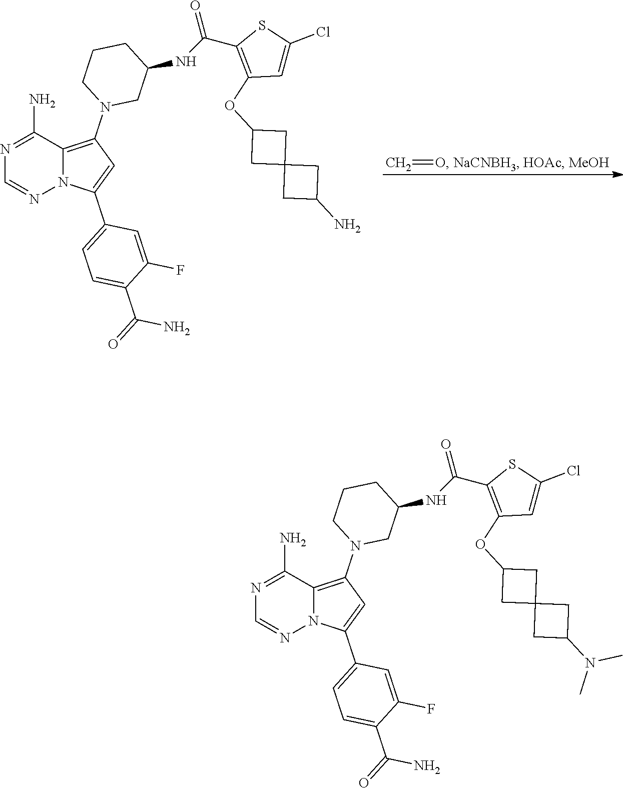

Example 944C (30 mg, 0.042 mmol) was dissolved in MeOH (500 μL) and to this mixture was added formaldehyde solution (37 wt. % in H₂O, containing 10-15% Methanol as stabilizer, 8.2 mg, 0.10 mmol), sodium cyanoborohydride (9.3 mg, 0.15 mmol), and acetic acid (9.7 μL, 0.17 mmol). The mixture was stirred overnight at rt. The reaction was worked up by evaporating most of the methanol and partitioned between EtOAc and 1:1 water/brine. The organic layer was evaporated, and the resultant crude product was purified by preparative HPLC to afford Example 944 (18.9 mg, 67% yield). LC-MS Method B: RT=1.23 min, [M+H]⁺=667.2, LC-MS Method A: RT=1.59 min. [M+H]⁺=667.1; ¹H NMR (500 MHz, DMSO-d₆) δ 8.08 (br d, J=13.0 Hz, 1H), 8.01-7.89 (m, 2H), 7.79-7.73 (m, 1H), 7.72-7.67 (m, 1H), 7.66-7.61 (m, 1H), 7.52-7.32 (m, 1H), 7.28-7.23 (m, 2H), 7.18-7.03 (m, 2H), 4.94-4.73 (m, 1H), 4.33-4.08 (m, 1H), 3.64-3.45 (m, 2H), 3.24-3.09 (m, 1H), 3.05-2.82 (m, 2H), 2.69-2.52 (m, 8H), 2.40-2.30 (m, 1H), 2.24-2.06 (m, 5H), 1.92-1.75 (m, 3H), 1.69-1.54 (m, 1H).

### Example 945

### Preparation of N—((R)-1-(4-Amino-7-(4-carbamoyl-3-fluorophenyl)pyrrolo[2,1-f][1,2,4]triazin-5-yl)piperidin-3-yl)-2-methyl-4-(((cis)-4-((neopentylamino)methyl)cyclohexyl)oxy)thiazole-5-carboxamide

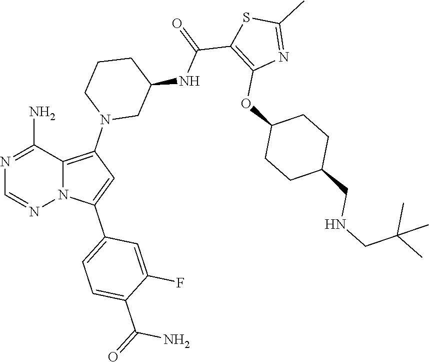 [SMILES: Cc1nc(O[C@H]2CC[C@@H](CNCC(C)(C)C)CC2)c(C(=O)N[C@@H]2CCCN(c3cc(-c4ccc(C(N)=O)c(F)c4)n4ncnc(N)c34)C2)s1]

### Example 945A. Ethyl 4-(((cis)-4-(((tert-butoxycarbonyl)amino)methyl)cyclohexyl)oxy)-2-methylthiazole-5-carboxylate

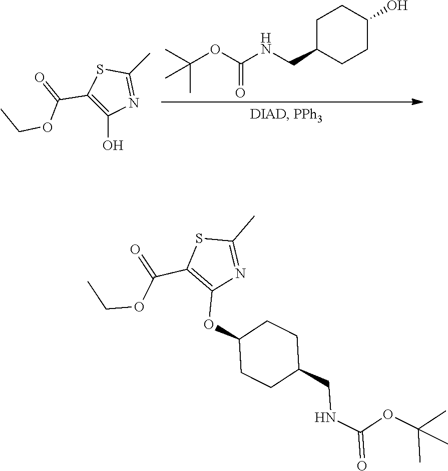

Triphenylphosphine (1.05 g, 4.01 mmol), and DIAD (0.779 mL, 4.01 mmol) were mixed and stirred in in THF (20 mL) for 0.5 h. Methyl 5-chloro-3-hydroxythiophene-2-carboxylate (0.500 g, 2.67 mmol) and tert-butyl (((trans)-4-hydroxycyclohexyl)methyl)carbamate (0.612 g, 2.67 mmol), were then added and the mixture was stirred at rt overnight. The following day, more triphenylphosphine (1.051 g, 4.01 mmol), and DIAD (0.779 mL, 4.01 mmol) were added. The following day, the reaction was evaporated, and the residue purified by flash chromatography (20-25% EtOAc/hexanes gradient), followed by a second column (10-20% EtOAc/hexanes gradient) to yield 0.629 g of Example 945A as a yellow oil. MS: (M+H)⁺=399.1.

### Example 945B. 4-(((cis)-4-(((tert-butoxycarbonyl)amino)methyl)cyclohexyl)oxy)-2-methylthiazole-5-carboxylic acid

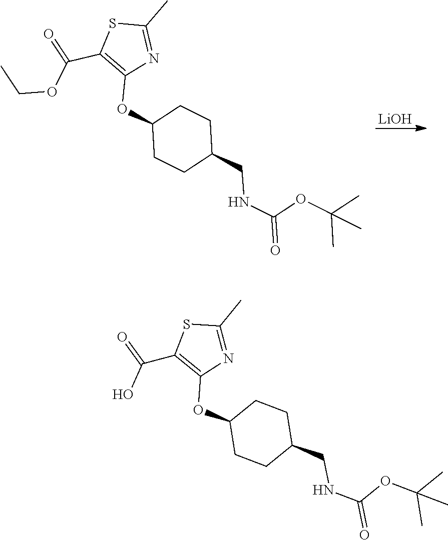 [SMILES: CCOC(=O)c1sc(C)nc1O[C@H]1CC[C@@H](CNC(=O)OC(C)(C)C)CC1 ; "LiOH" ; Cc1nc(O[C@H]2CC[C@@H](CNC(=O)OC(C)(C)C)CC2)c(C(=O)O)s1]

Example 945A (the entire amount from the previous step) was dissolved in MeOH (2 mL) and to it was added 2N lithium hydroxide (3.34 mL, 6.68 mmol). The mixture was stirred at rt for 4 h, LC-MS. The MeOH was evaporated and the aqueous acidified to pH=1. The aqueous was extracted with EtOAc (3×), the organic layers were collected and washed with brine, dried (sodium sulfate) and evaporated to yield 450.5 mg of Example 945B as a light pink solid. MS: (M+Na)⁺=393.1; (M+K)⁺=409.0.

### Example 945C. N—((R)-1-(4-Amino-7-(4-carbamoyl-3-fluorophenyl)pyrrolo[2,1-f][1,2,4]triazin-5-yl)piperidin-3-yl)-4-(((cis)-4-(aminomethyl)cyclohexyl)oxy)-2-methylthiazole-5-carboxamide, 2HCl salt

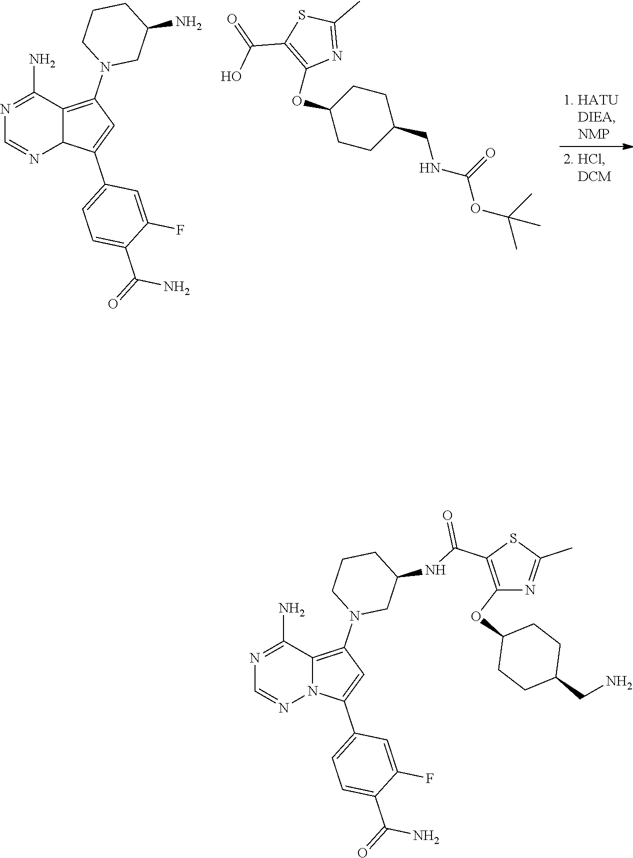 [SMILES: NC(=O)c1ccc(C2=CC(N3CCC[C@@H](N)C3)=C3C(N)=NC=NC23)cc1F ; Cc1nc(O[C@H]2CC[C@@H](CNC(=O)OC(C)(C)C)CC2)c(C(=O)O)s1 ; Cc1nc(O[C@H]2CC[C@@H](CN)CC2)c(C(=O)N[C@@H]2CCCN(c3cc(-c4ccc(C(N)=O)c(F)c4)n4ncnc(N)c34)C2)s1]

To a solution of Example 139D, HCl salt (426 mg, 0.964 mmol). Example 945B (357.2 mg, 0.964 mmol), and DIEA (0.842 mL, 4.82 mmol) in DMF, was added HATU (440 mg, 1.157 mmol). The mixture was stirred at rt for 4 d, then was diluted with EtOAc. The organic phase was washed with 10% LiCl (3×) and brine. The organic phase was dried (sodium sulfate) and evaporated. The crude product was dried onto Celite from MeOH, then was purified by flash chromatography (solid loading from Celite, 50-100% EtOAc/hexanes gradient, followed by 10% MeOH in DCM to yield 554.2 mg of a viscous brown oil. MS: (M+H)⁺=722.2. The Boc-protected intermediate was dissolved in DCM (5 mL) and 4M HCl in dioxane (2.41 mL, 9.64 mmol) was added. A minimum amount of MeOH (approx. 5 mL) was added to dissolve the precipitate that had formed. The mixture was stirred at rt overnight, then was evaporated. The residue was co-evaporated 3× from MeOH to yield Example 945C (520.7 mg) as a yellow brown amorphous solid. MS: (M+H)⁺=622.2.

### Example 945

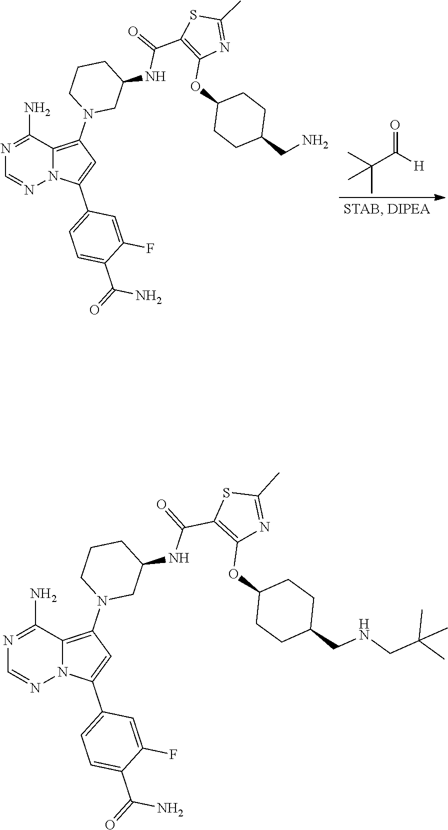

Example 945C (19.7 mg, 0.028 mmol) was stirred in DCM (1 mL) and to this mixture was added DIEA (0.020 mL, 0.113 mmol), trimethylacetaldehyde (2.443 mg, 0.028 mmol) and lastly sodium triacetoxyborohydride (9.02 mg, 0.043 mmol). The mixture was stirred at it overnight. The reaction was evaporated, then the residue was purified by preparative HPLC to afford Example 945 (3.8 mg, 19% yield). LC-MS Method B: RT=1.29 min, [M+H]⁺=692.0; LC-MS Method A: RT=1.58 min, [M+H]⁺=692.1; ¹H NMR (500 MHz, DMSO-d6) 8.14-8.09 (m, 1H), 8.03-7.96 (m, 1H), 7.93-7.87 (m, 1H), 7.79-7.70 (m, 1H), 7.68-7.58 (m, 2H), 7.47-7.24 (m, 1H), 7.23-7.16 (m, 1H), 5.29-5.18 (m, 1H), 4.28-4.17 (m, 1H), 2.64-2.57 (m, 3H), 2.29-2.09 (m, 2H), 2.05-1.78 (m, 12H), 1.67-1.36 (m, 7H), 1.24-1.09 (m, 3H), 0.77-0.66 (m, 9H).

### Example 946

### Preparation of N—((R)-1-(7-(1-acetyl-2,5-dihydro-1H-pyrrol-3-yl)-4-aminopyrrolo[2,1-f][1,2,4]triazin-5-yl)piperidin-3-yl)-4-((1-ethyl-2,2-dimethylpiperidin-4-yl)oxy)-2-methylthiazole-5-carboxamide

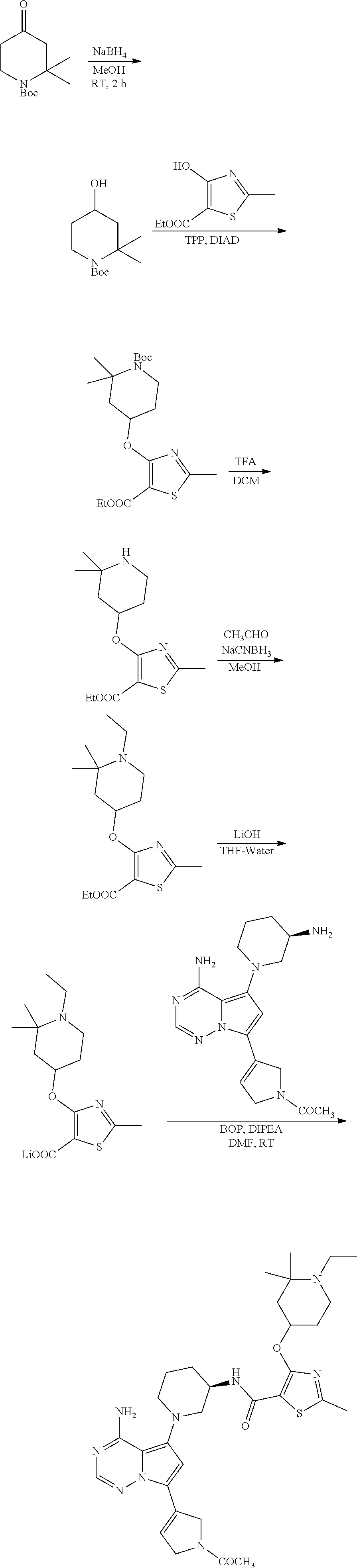

### Example 946A. tert-butyl 4-hydroxy-2,2-dimethylpiperidine-1-carboxylate

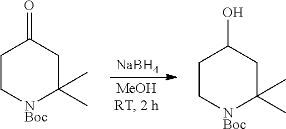

To a stirred solution of tert-butyl 2,2-dimethyl-4-oxopiperidine-1-carboxylate (20 g, 88 mmol) in MeOH (200 mL) was added NaBH₄ (6.66 g, 176 mmol) at 0° C. and the mixture was allowed to stir for 1 h. It was concentrated and partitioned between DCM and aq. NH₄Cl solution. Organic layer was collected, dried over sodium sulphate, filtered, and concentrated to afford Example 946A (19.83 g, 98%) as gummy liquid; ¹H NMR (400 MHz, DMSO-d₆) δ ppm 4.63 (d, J=4.50 Hz, 1H), 3.78-3.57 (m, 2H), 3.15-3.00 (m, 1H), 1.89-1.76 (m, 1H), 1.68-1.57 (m, 1H), 1.43 (s, 3H), 1.41-1.35 (m, 9H), 1.32-1.25 (m, 1H), 1.23 (s, 3H).

### Example 946B. Ethyl 4-((1-(tert-butoxycarbonyl)-2,2-dimethylpiperidin-4-yl)oxy)-2-methylthiazole-5-carboxylate

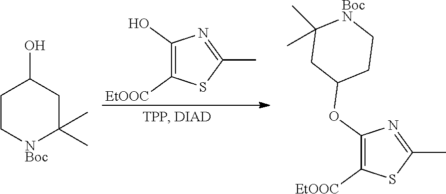

To a stirred solution of ethyl 4-hydroxy-2-methylthiazole-5-carboxylate (12 g, 64.1 mmol), Example 946A (19.11 g, 83 mmol) and triphenylphosphine (25.2 g, 96 mmol) in THF (110 mL) was added DIAD (18.7 mL, 96 mmol) and the mixture was stirred at RT for 4 h. It was concentrated in vacuum and the residue obtained was purified by flash chromatography (0% to 20% EtOAc/hexanes gradient) to yield Example 946B as racemate. The racemate was purified by preparative SFC (Chiralcel OJ-H) to get First eluting isomer (Iso-1) (8.56 g, 34%, pale yellow liquid: SFC Method: RT=4.72 min; 100% cc: MS: [M+H]⁺=399.35) and Second eluting isomer (Iso-2) (9.61 g, 38%, pale yellow liquid; SFC Method: RT=5.28 min; 94.2% ee; MS: [M+H]⁺=399.35). The first eluting isomer (Iso-1) was taken to next step.

### Example 946C. Ethyl 4-((2,2-dimethylpiperidin-4-yl)oxy)-2-methylthiazole-5-carboxylate

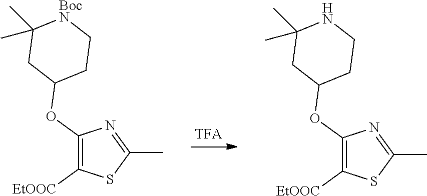

To a stirred solution of Example 946B (2 g, 5.02 mmol) in DCM (20 mL) was added TFA (5.80 mL, 75 mmol) and the reaction mixture was stirred at RT for 4 h. Concentrated in vacuum to yield Example 946C (1.2 g, 80%) as a yellow solid. MS: [M+H]⁺=299.2.

### Example 946D. Ethyl 4-((1-ethyl-2,2-dimethylpiperidin-4-yl)oxy)-2-methylthiazole-5-carboxylate

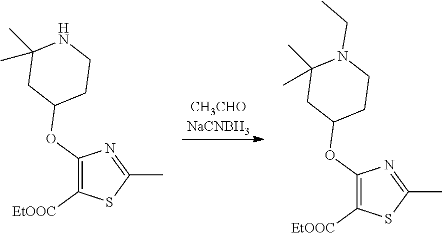

To a stirred solution of Example 946C, TFA (1.5 g, 3.64 mmol), acetic acid (0.416 mL, 7.27 mmol) and acetaldehyde (0.411 mL, 7.27 mmol) in MeOH (16 mL) was added sodium cyanoborohydride (0.457 g, 7.27 mmol) and the reaction mixture was stirred at RT for 4 h. It was concentrated and partitioned between DCM and saturated ammonium chloride. The organic phase was separated then the aqueous phase was extracted with DCM (3×15 mL). The combined organic phase was dried over Na₂SO₄, filtered and concentrated. The residue was purified by flash chromatography (0% to 5% MeOH/DCM gradient) to yield Example 946D (1 g, 84%) as a brown oil. MS: [M+H]⁺=326.2.

### Example 946E. Lithium 4-((1-ethyl-2,2-dimethylpiperidin-4-yl)oxy)-2-methylthiazole-5-carboxylate

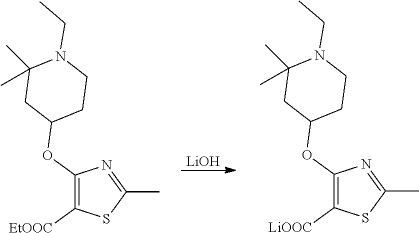

To a stirred solution of Example 946D (2 g, 6.13 mmol) in a mixture of THF (9 mL), MeOH (7 mL) and water (3 mL) was added LiOH·H₂O (0.293 g, 12.25 mmol) and the reaction mixture was stirred at RT for 6 h. The mixture was concentrated to afford Example 946E (1.5 g, 80%) which was used directly for the next step. MS: [M+H]⁺=299.2.

### Example 946

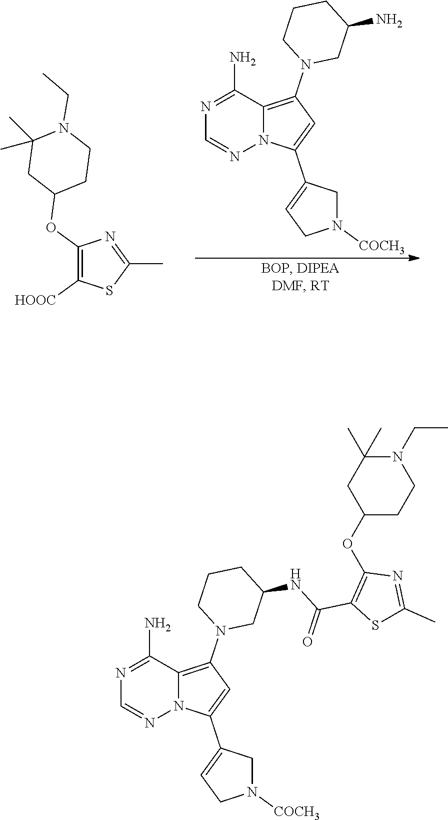

To a stirred solution of Intermediate 32 and Example 946E (4.2 mg, 0.033 mmol) in DMF (0.5 mL) were added DIEA (0.028 mL, 0.16 mmol) and HATU (13.2 mg, 0.035 mmol) and the mixture was stirred at RT for 2.5 h. Filtered and the filtrate was concentrated and purified by preparative HPLC to yield Example 946 (93 mg, 17%). LC-MS Method E: RT=1.32 min, [M+H]⁺=622.6; ¹H NMR (400 MHz, DMSO-d₆) δ ppm 7.87 (d, J=1.9 Hz, 2H), 7.33 (br s, 1H), 6.85-6.61 (m, 3H), 5.13-4.95 (m, 1H), 4.69 (br d, J=1.8 Hz, 1H), 4.48 (br d, J=8.1 Hz, 2H), 4.27 (br d, J=2.3 Hz, 1H), 4.21-4.04 (m, 1H), 3.13 (br s, 1H), 2.90 (br s, 4H), 2.61 (s, 3H), 2.20-1.98 (m, 5H), 1.95-1.72 (m, 4H), 1.60 (br s, 2H), 1.21-0.79 (m, 1H).

### Example 947

### Preparation of N—((R)-1-(4-amino-7-(4-carbamoyl-3-fluorophenyl)pyrrolo[2,1-f][1,2,4]triazin-5-yl)piperidin-3-yl)-5-(difluoromethyl)-3-(((R)-1-ethyl-2,2-dimethylpiperidin-4-yl)oxy)thiophene-2-carboxamide

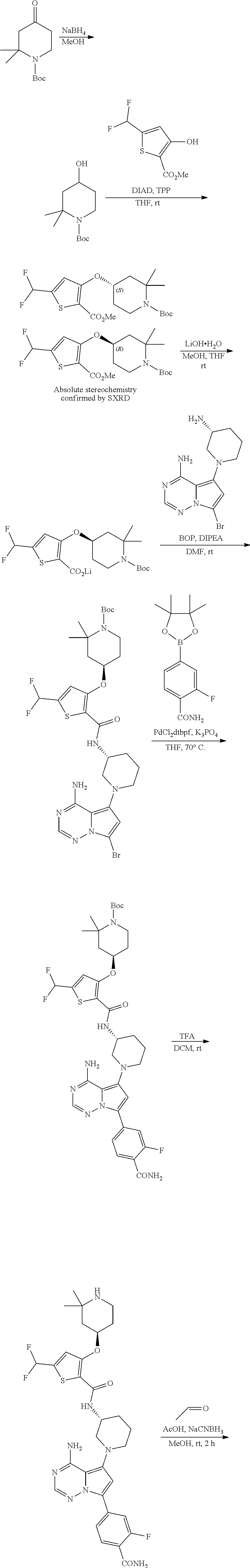

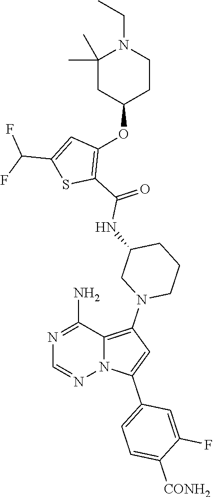 [SMILES: CCN1CC[C@@H](Oc2cc(C(F)F)sc2C(=O)N[C@@H]2CCCN(c3cc(-c4ccc(C(N)=O)c(F)c4)n4ncnc(N)c34)C2)CC1(C)C]

### Example 947A. tert-butyl 4-hydroxy-2,2-dimethylpiperidine-1-carboxylate

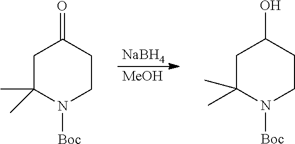

Analogous to the preparation of Example 946A, reaction of tert-buty 2,2-dimethyl-4-oxopiperidine-1-carboxylate (20 g, 88 mmol) and NaBH₄ (6.66 g, 176 mmol) afforded Example 947A (19.83 g, 98%) as transparent gummy liquid. ¹H NMR (400 MHz, DMSO-d₆) δ ppm 4.63 (d, J=4.5 Hz, 1H), 3.76-3.60 (m, 2H), 3.08 (ddd, J=3.8, 10.1, 13.6 Hz, 1H), 1.83 (m, J=1.5, 3.6, 11.3, 13.1 Hz, 1H), 1.69-1.49 (m, 1H), 1.45-1.35 (m, 12H), 1.32-1.17 (m, 4H).

### Example 947B. tert-butyl (R)-4-((5-(difluoromethyl)-2-(methoxycarbonyl)thiophen-3-yl)oxy)-2,2-dimethylpiperidine-1-carboxylate

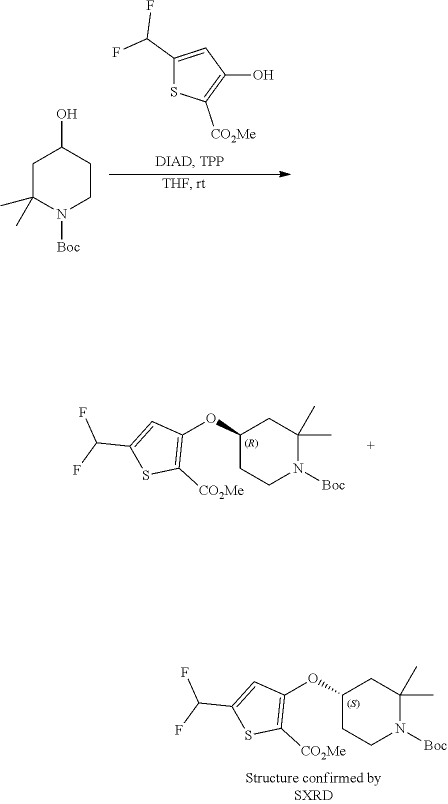

Analogous to the preparation of Example 946B, reaction of methyl 5-(difluoro methyl)-3-hydroxythiophene-2-carboxylate (1.0 g, 4.80 mmol), Example 947A (1.43 g, 6.24 mmol), triphenylphosphine (1.890 g, 7.21 mmol) and DIAD (1.401 mL, 7.21 mmol) in THF (10 mL) afforded crude, Example 947B which was purified by preparative SFC (Chiralcel OJ-H) to get two isomers.

First eluting isomer (Iso-1): 480 mg, 24%, as a white crystal solid; SFC Method: RT=5.15 min; 99.4% ee: MS: [M+H]⁺=420.20; ¹H NMR (400 MHz, DMSO-d₆) δ ppm 7.55 (s, 1H), 7.43-7.10 (m, 1H), 4.80-4.73 (m, 1H), 3.76 (s, 3H), 3.62-3.55 (m, 1H), 3.42-3.36 (m, 1H), 2.13-2.05 (m, 1H), 1.92 (dd, J=4.5, 13.5 Hz, 1H), 1.79 (dd, J=8.3, 13.8 Hz, 1H), 1.65-1.56 (m, 1H), 1.47 (s, 3H), 1.43-1.35 (m, 12H).

Second eluting isomer (Iso-2): 527 mg, 26% as a white crystal solid; SFC Method: RT=5.92 min; 94.2% ee; MS: [M+H]⁺=420.20; ¹H NMR (400 MHz, DMSO-d₆) δ ppm 7.55 (s, 1H), 7.42-7.06 (m, 1H), 4.80-4.73 (m, 1H), 3.76 (s, 3H), 3.66-3.48 (m, 1H), 3.43-3.38 (m, 1H), 2.09 (tdd, J=4.6, 8.7, 13.1 Hz, 1H), 1.92 (dd, J=4.5, 13.5 Hz, 1H), 1.79 (dd, J=8.3, 13.8 Hz, 1H), 1.65-1.53 (m, 1H), 1.47 (s, 3H), 1.43-1.36 (m, 12H). Absolute stereochemistry of the isomers was confirmed by single crystal X-ray diffraction (SXRD). First eluting isomer (Iso-1) was taken for next step.

### Example 947C. Lithium (R)-3-((1-(tert-butoxycarbonyl)-2,2-dimethylpiperidin-4-yl)oxy)-5-(difluoromethyl)thiophene-2-carboxylate

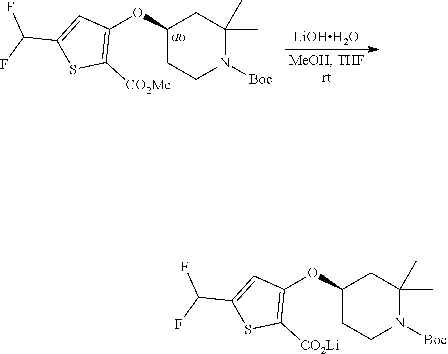

Analogous to the preparation of Example 946E, starting with Example 947B (380 mg, 0.906 mmol) and LiOH·H₂O (43.4 mg, 1.81 mmol) in THF, MeOH and water afforded Example 947C (350 mg, 94%) as a light orange solid. MS: [M+H]⁺=306.15; ¹H NMR (400 MHz, DMSO-d₆) δ ppm 7.28-7.13 (m, 1H), 7.05 (d, J=2.0 Hz, 1H), 4.83-4.76 (m, 1H), 3.62 (ddd, J=4.3, 7.6, 13.6 Hz, 1H), 3.28-3.21 (m, 1H), 1.96 (br d, J=4.5 Hz, 1H), 1.83 (dd, J=4.8, 13.3 Hz, 1H), 1.69 (dd, J=9.3, 13.3 Hz, 1H), 1.59-1.49 (m, 1H), 1.46 (s, 3H), 1.40 (s, 9H), 1.29 (s, 3H).

### Example 947D. tert-butyl (R)-4-((2-(((R)-1-(4-amino-7-bromopyrrolo[2,1-f][1,2,4]triazin-5-yl)piperidin-3-yl)carbamoyl)-5-(difluoromethyl)thiophen-3-yl)oxy)-2,2-dimethylpiperidine-1-carboxylate

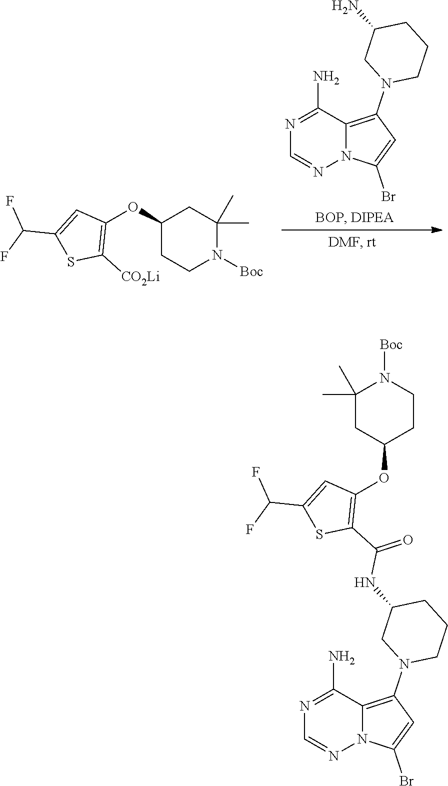

Analogous to the preparation of Example 946, coupling Intermediate 61 with Example 947C (450 mg, 1.09 mmol) using BOP (581 mg, 1.31 mmol) and DIEA (0.955 mL, 5.47 mmol) afforded Example 947D (600 mg, 79%) as a brown gummy liquid. MS: [M+H]⁺=698.00; ¹H NMR (400 MHz, CHCl₃-d) δ ppm 8.04 (s, 1H), 7.05-6.73 (m, 1H), 6.62-6.55 (m, 1H), 4.69-4.52 (m, 1H), 3.91-3.66 (m, 1H), 3.37 (ddd, J=3.8, 9.5, 13.8 Hz, 1H), 2.97 (s, 3H), 2.90 (s, 3H), 2.66 (d, J=9.0 Hz, 9H), 2.01 (br dd, J=3.5, 13.5 Hz, 1H), 1.89 (br d, J=6.0 Hz, 1H), 1.86-1.69 (m, 2H), 1.50-1.28 (m, 9H), 0.95-0.81 (m, 1H).

### Example 947E. tert-butyl (R)-4-((2-(((R)-1-(4-amino-7-(4-carbamoyl-3-fluorophenyl)pyrrolo[2,1-f][1,2,4]triazin-5-yl)piperidin-3-yl)carbamoyl)-5-(difluoromethyl)thiophen-3-yl)oxy)-2,2-dimethylpiperidine-1-carboxylate

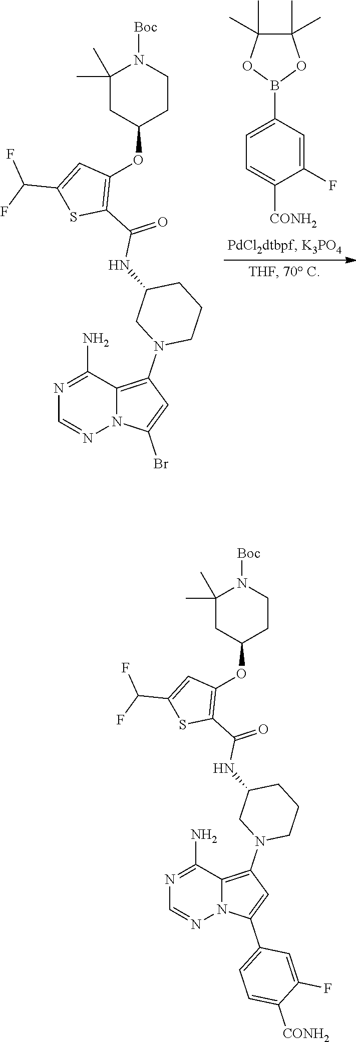 [SMILES: CC(C)(C)OC(=O)N1CC[C@@H](Oc2cc(C(F)F)sc2C(=O)N[C@@H]2CCCN(c3cc(Br)n4ncnc(N)c34)C2)CC1(C)C ; CC1(C)OB(c2ccc(C(N)=O)c(F)c2)OC1(C)C ; CC(C)(C)OC(=O)N1CC[C@@H](Oc2cc(C(F)F)sc2C(=O)N[C@@H]2CCCN(c3cc(-c4ccc(C(N)=O)c(F)c4)n4ncnc(N)c34)C2)CC1(C)C]

To a stirred solution of Example 947D (200 mg, 0.286 mmol), 2-fluoro-4-(4,4,5,5-tetramethyl-1,3,2-dioxaborolan-2-yl)benzamide (114 mg, 0.429 mmol) and Potassium phosphate, tribasic (182 mg, 0.859 mmol) in THF/Water (3:1) was added [1,1′-bis(di-tert-butylphosphino)ferrocene]dichloropalladium(II) (11.2 mg, 0.017 mmol) and the mixture was heated at 70° C. for 6 h. The reaction mixture was cooled to rt, concentrated and partitioned between ethyl acetate (15 mL) and brine (20 mL). Organic layer was collected, dried over sodium sulphate, filtered and concentrated to afford crude product which was purified by silica-gel column to afford Example 947E (119 mg, 55%). MS: [M+H]⁺=757.25; ¹H NMR (400 MHz, CHCl₃-d) δ ppm 8.23-8.09 (m, 2H), 7.94 (s, 1H), 7.86-7.79 (m, 1H), 6.89 (m, 1H), 5.83-5.73 (m, 1H), 4.70-4.57 (m, 1H), 4.42-4.31 (m, 1H), 3.92-3.79 (m, 1H), 3.44-3.33 (m, 1H), 3.19-3.08 (m, 1H), 2.35-2.26 (m, 1H), 2.07-1.75 (m, 8H), 1.49-1.46 (m, 3H), 1.43-1.39 (m, 4H), 1.28-1.24 (m, 13H).

### Example 947F. N—((R)-1-(4-amino-7-(4-carbamoyl-3-fluorophenyl)pyrrolo[2,1-f][1,2,4]triazin-5-yl)piperidin-3-yl)-5-(difluoromethyl)-3-(((R)-2,2-dimethylpiperidin-4-yl)oxy)thiophene-2-carboxamide

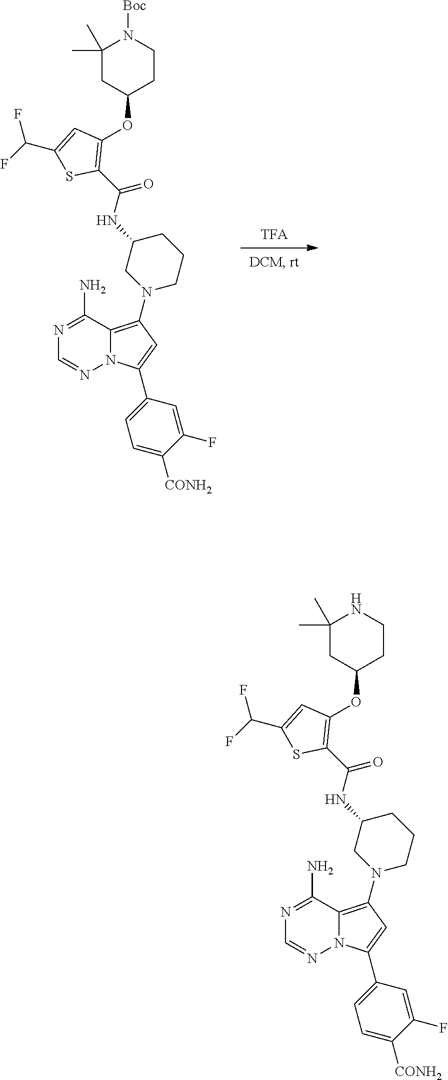 [SMILES: CC(C)(C)OC(=O)N1CC[C@@H](Oc2cc(C(F)F)sc2C(=O)N[C@@H]2CCCN(c3cc(-c4ccc(C(N)=O)c(F)c4)n4ncnc(N)c34)C2)CC1(C)C ; "DCM, rt" ; "TFA" ; CC1(C)C[C@H](Oc2cc(C(F)F)sc2C(=O)N[C@@H]2CCCN(c3cc(-c4ccc(C(N)=O)c(F)c4)n4ncnc(N)c34)C2)CCN1]

Analogous to the preparation of Example 946C, starting with Example 947E (100 mg, 0.132 mmol) and TFA (0.153 mL, 1.98 mmol) in DCM (1.5 mL) afforded Example 947F (70 mg, 81%) as light brown solid. MS: [M+H]⁺=657.25.

### Example 947

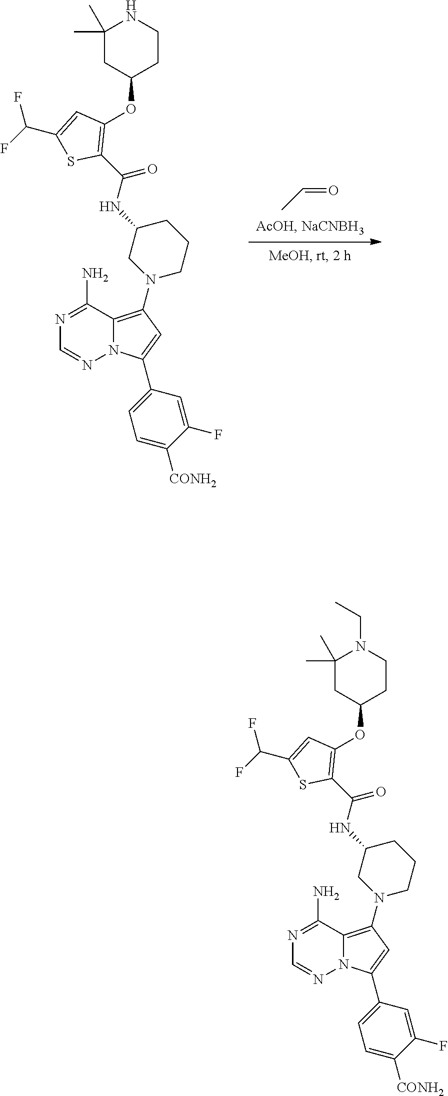

Analogous to the preparation of Example 946D, reaction of Example 947F (40 mg, 0.061 mmol), sodium cyanoborohydride (7.7 mg, 0.12 mmol), acetaldehyde (6.9 μl, 0.12 mmol) and acetic acid (3.5 μl, 0.061 mmol) in MeOH (1 mL) afforded crude product which was purified by preparative HPLC to get Example 947 (9.7 mg, 24%) as pale-yellow solid. LC-MS Method E: RT=1.53 min, [M+H]⁺=685.3; LC-MS Method F: RT=1.08 min, [M+H]-=685.3; ¹H NMR (400 MHz, DMSO-d₆) δ ppm 8.18-8.10 (m, 2H), 8.03-7.95 (m, 2H), 7.93-7.83 (m, 1H), 7.66 (s, 2H), 7.63 (s, 2H), 7.26 (s, 2H), 4.89-4.60 (m, 2H), 4.29-4.14 (m, 2H), 3.31-3.30 (m, 3H), 3.06-2.88 (m, 1H), 2.82-2.71 (m, 1H), 2.56-2.53 (m, 3H), 1.85 (br s, 4H), 1.24 (s, 6H), 0.97 (br s, 6H).

### Example 948

### N—((R)-1-(7-(1-acetyl-2,5-dihydro-1H-pyrrol-3-yl)-4-aminopyrrolo[2,1-f][1,2,4]triazin-5-yl)piperidin-3-yl)-5-chloro-3-((1,2,2-trimethylpiperidin-4-yl)oxy)thiophene-2-carboxamide

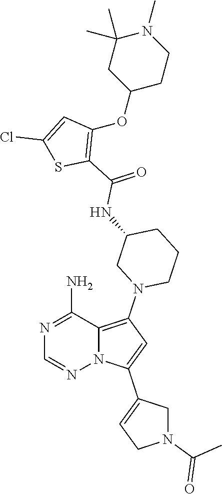 [SMILES: CC(=O)N1CC=C(c2cc(N3CCC[C@@H](NC(=O)c4sc(Cl)cc4OC4CCN(C)C(C)(C)C4)C3)c3c(N)ncnn23)C1]

Example 948 was prepared using the same procedure as shown in Example 947. Analogous to synthesis of Example 947B, two stereoisomers (Intermediate 1) were separated in second step and isomer 1 (Iso-1) was taken for remaining steps to get Example 948.

### Intermediate 1. tert-Butyl 4-((2-(methoxycarbonyl)-5-methylthiophen-3-yl)oxy)-2,2-dimethylpiperidine-1-carboxylate

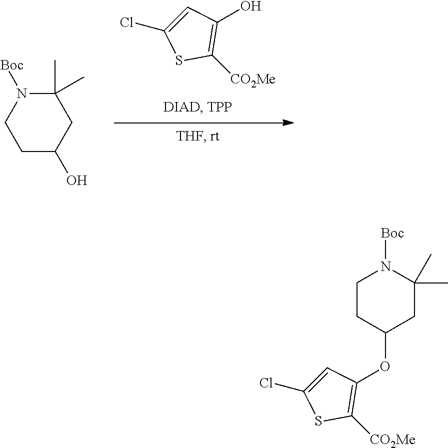

Analogous to the preparation of Example 946D, reaction of methyl 5-chloro-3-hydroxythiophene-2-carboxylate (0.8 g, 4.15 mmol). tert-butyl 4-hydroxy-2,2-dimethylpiperidine-1-carboxylate (0.952 g, 4.15 mmol), triphenylphosphine (1.416 g, 5.40 mmol) and DIAD (0.808 mL, 4.15 mmol) afforded racemate (MS: [M-Boc+2H]⁺=304.0) as a yellow thick liquid, which was purified by preparative SFC (Chiralcel ODH) to get two chiral isomers.

First eluting isomer (Iso-1): 500 mg, 30%, yellow thick liquid: SFC Method: RT=4.51 min; 100% cc. MS: [M+H]⁺=403.8; ¹H NMR (400 MHz, CHCl₃-d) δ ppm 6.71 (s, 1H), 4.56-4.49 (m, 1H), 3.83 (s, 3H), 3.79-3.69 (m, 1H), 3.55-3.47 (m, 1H), 2.29-2.04 (m, 1H), 2.00-1.81 (m, 3H), 1.57 (d, J=5.0 Hz, 6H), 1.52-1.48 (m, 9H), 145-1.41 (m, 3H).

Second eluting Isomer (Iso-2): 400 mg, 24%, yellow thick liquid, SFC Method: RT=5.12 min; 95.5% cc; [M+H]⁺=403.8; ¹H NMR (400 MHz, CHCl₃-d) δ ppm 6.71 (s, 1H), 4.56-4.49 (m, 1H), 3.83 (s, 3H), 3.79-3.69 (m, 1H), 3.55-3.47 (m, 1H), 2.29-2.04 (m, 1H), 2.00-1.81 (m, 3H), 1.57 (d, J=5.0 Hz, 6H), 1.52-1.48 (m, 9H), 1.45-1.41 (m, 3H).

Example 948. LC-MS Method E: RT=1.37 min, [M+H]⁺=627.3; LC-MS Method F: RT=1.03 min, [M+H]⁺=627.3; ¹H NMR (400 MHz, DMSO-d₆) δ ppm 7.87 (d, J=2.0 Hz, 1H), 7.41 (d, J=1.5 Hz, 1H), 6.73 (d, J=13.3 Hz, 2H), 4.71-4.58 (m, 2H), 4.52-4.44 (m, 2H), 4.29-4.25 (m, 1H), 4.14 (br s, 2H), 3.14-3.09 (m, 1H), 2.89 (br s, 3H), 2.11 (br dd, J=2.9, 4.9 Hz, 2H), 2.03 (d, J=16.3 Hz, 4H), 1.90-1.73 (m, 4H), 1.53 (br s, 3H), 0.96 (br s, 6H).

### Example 949

### Preparation of N—((R)-1-(4-amino-7-(3-fluoro-4-(1H-tetrazol-1-yl)phenyl)pyrrolo[2,1-f][1,2,4]triazin-5-yl)piperidin-3-yl)-4-((2-isopropyl-2-azabicyclo[2.2.1]heptan-5-yl)oxy)-2-methylthiazole-5-carboxamide

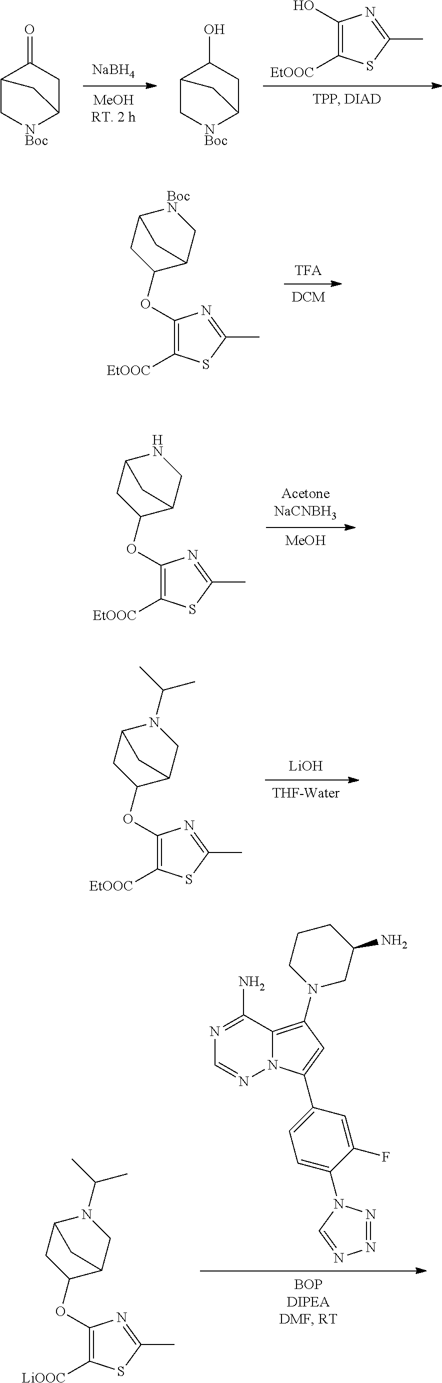

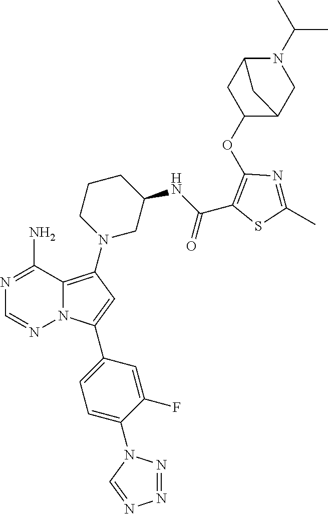 [SMILES: Cc1nc(OC2CC3CC2CN3C(C)C)c(C(=O)N[C@@H]2CCCN(c3cc(-c4ccc(-n5cnnn5)c(F)c4)n4ncnc(N)c34)C2)s1]

### Example 949A. tert-Butyl 5-hydroxy-2-azabicyclo[2.2.1]heptane-2-carboxylate

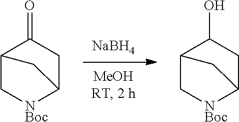 [SMILES: CC(C)(C)OC(=O)N1CC2CC1CC2=O ; "MeOHRT, 2 h" ; "NaBH4" ; CC(C)(C)OC(=O)N1CC2CC1CC2O]

To a stirred solution of tert-butyl 5-oxo-2-azabicyclo[2.2.1]heptane-2-carboxylate (10 g, 47.3 mmol) in MeOH (200 mL) at 0° C. was added NaBH₄ (3.58 g, 95 mmol) and the reaction mixture was stirred at 0° C. for 2 h. The reaction was quenched with saturated ammonium chloride solution (100 mL), concentrated and partitioned between water (500 mL) and ethyl acetate (500 mL). The organic layer was separated, dried over sodium sulphate and concentrated under reduced pressure to afford Example 949A (10 g, 99%) as a yellow solid.

### Example 949B. Ethyl 4-((2-(tert-butoxycarbonyl)-2-azabicyclo[2.2.1]heptan-5-yl)oxy)-2-methylthiazole-5-carboxylate

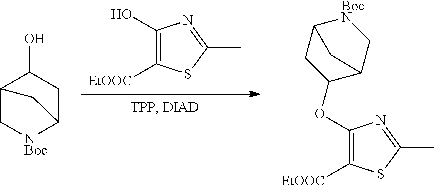

To a stirred solution of ethyl 4-hydroxy-2-methylthiazole-5-carboxylate (5.5 g, 29.4 mmol), tert-butyl 5-hydroxy-2-azabicyclo[2.2.1]heptane-2-carboxylate (6.27 g, 29.4 mmol) and triphenylphosphine (15.41 g, 58.8 mmol) in THF (120 mL) was added DIAD (9.71 mL, 49.9 mmol) and it was stirred for 4 h at rt. The reaction mixture was concentrated in vacuum, and the residue was purified by flash column chromatography (0% to 20% EtOAc/hexanes gradient) to afford Example 949B as racemate. The racemate was purified by preparative SFC (Lux cellulose C2). The second eluting isomer (Iso-2) was taken to next step. First eluting isomer (Iso-1): 3.2 g, 28.5%, pale yellow liquid; SFC Method: RT=5.49 min; 94.4% ee; MS: [M−^{t}Bu]⁺=327.0. Second eluting isomer (Iso-2): 3.3 g, 29.4%, yellow oil; SFC Method: RT=6.34 min; 98.5% ee; MS: [M−^{t}Bu]⁺=327.0.

### Example 949C. Ethyl 4-((2-azabicyclo[2.2.1]heptan-5-yl)oxy)-2-methylthiazole-5-carboxylate

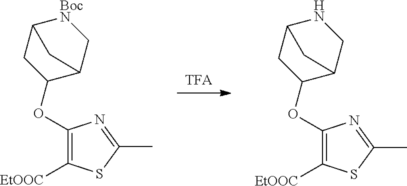 [SMILES: CCOC(=O)c1sc(C)nc1OC1CC2CC1CN2C(=O)OC(C)(C)C ; CCOC(=O)c1sc(C)nc1OC1CC2CC1CN2 ; "TFA"]

To a stirred solution of ethyl 4-((2-tert-butoxycarbonyl)-2-azabicyclo[2.2.1]heptan-5-yl)oxy)-2-methylthiazole-5-carboxylate (Iso-2, 3 g, 7.84 mmol) in DCM (30 mL) was added TFA (9.06 mL, 118 mmol) and the reaction mixture was stirred at RT for 2 h. The residue was co-evaporated with DCM/MeOH to afford Example 949C (2.1 g, 95%) as a yellow semisolid, which was taken up for next step without further purification. MS: [M+H]⁺=283.0.

### Example 949D. Ethyl 4-((2-isopropyl-2-azabicyclo[2.2.1]heptan-5-yl)oxy)-2-methylthiazole-5-carboxylate

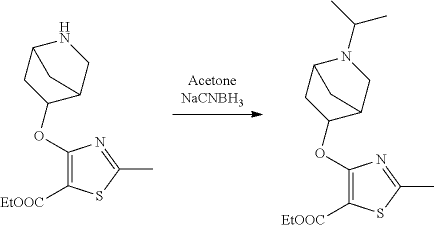 [SMILES: CCOC(=O)c1sc(C)nc1OC1CC2CC1CN2 ; "AcetoneNaCNBH3" ; CCOC(=O)c1sc(C)nc1OC1CC2CC1CN2C(C)C]

To a stirred solution of ethyl 4-((2-azabicyclo[2.2.1]heptan-5-yl)oxy)-2-methylthiazole-5-carboxylate (1.3 g, 4.60 mmol) in acetic acid (0.527 mL, 9.21 mmol), propan-2-one (5.35 g, 92 mmol) in MeOH (10 mL) was added followed by sodium cyanoborohydride (0.579 g, 9.21 mmol) and the reaction mixture was stirred at RT for 4 h. The mixture was concentrated and partitioned between DCM and saturated ammonium chloride. The aqueous phase was extracted with DCM (3×10 mL). The combined organic phase was dried over Na₂SO₄, filtered and concentrated. The residue was purified by flash chromatography (0% to 5% MeOH/DCM gradient) to yield Example 949D (1.4 g, 94%) as a brown oil. MS: [M+H]⁺=325.1.

### Example 949E. Lithium 4-((2-isopropyl-2-azabicyclo[2.2.1]heptan-5-yl)oxy)-2-methylthiazole-5-carboxylate

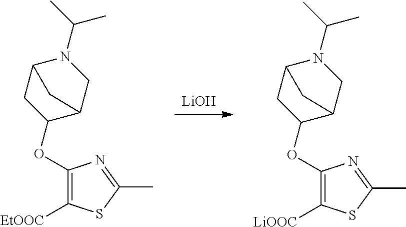

Analogous to the preparation of Example 946E, starting with ethyl 4-((2-isopropyl-2-azabicyclo[2.2.1]heptan-5-yl)oxy)-2-methylthiazole-5-carboxylate (1.4 g, 4.32 mmol) and LiOH (0.310 g, 12.95 mmol) yielded Example 949E (1.2 g, 94%) as an off-white solid. MS: [M+H]⁺=297.1.

### Example 949

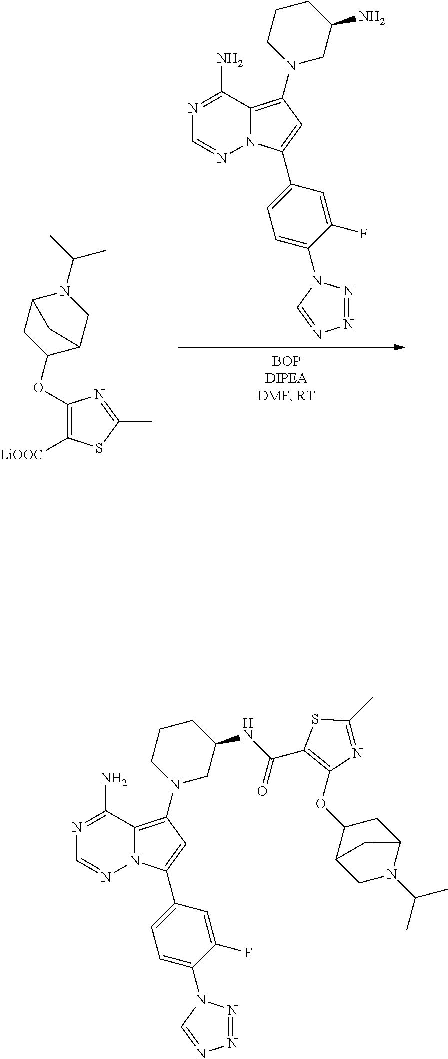

Analogous to the preparation of Example 946, reaction of Example 949E (676 mg, 2.28 mmol), (R)-5-(3-aminopiperidin-1-yl)-7-(3-fluoro-4-(1H-tetrazol-1-yl)phenyl)pyrrolo[2,1-f][1,2,4]triazin-4-amine (900 mg, 2.28 mmol), DIEA (1.12 mL, 6.85 mmol) and BOP (1 g, 2.28 mmol) in DMF (15 mL) yielded Example 949 (250 mg, 16%) as a pale yellow solid. LC-MS Method E: RT=1.88 min, [M+H]⁺=673.1; HPLC Method G: RT=4.59 min, Method H: RT=5.97 min; ¹H NMR (400 MHz, DMSO-d₆) δ ppm 10.04-9.91 (m, 1H), 8.51-8.40 (m, 1H), 8.31-8.24 (m, 1H), 8.22-8.04 (m, 1H), 8.01-7.90 (m, 2H), 7.34 (s, 2H), 6.95-6.64 (m, 1H), 4.96-4.85 (m, 1H), 4.22-4.08 (m, 1H), 3.25-3.14 (m, 1H), 3.05-2.78 (m, 4H), 2.70-2.65 (m, 1H), 2.62 (s, 3H), 2.38-2.21 (m, 3H), 1.84 (br s, 4H), 1.64-1.52 (m, 2H), 1.50-1.44 (m, 1H), 1.44-1.37 (m, 1H), 0.91 (dd, J=9.63, 6.13 Hz, 6H).

### Example 950

### Preparation of N—((R)-1-(4-Amino-7-(6-cyanopyridin-3-yl)pyrrolo[2,1-f][1,2,4]triazin-5-yl)piperidin-3-yl)-5-chloro-3-(((S)-quinuclidin-3-yl)oxy)thiophene-2-carboxamide

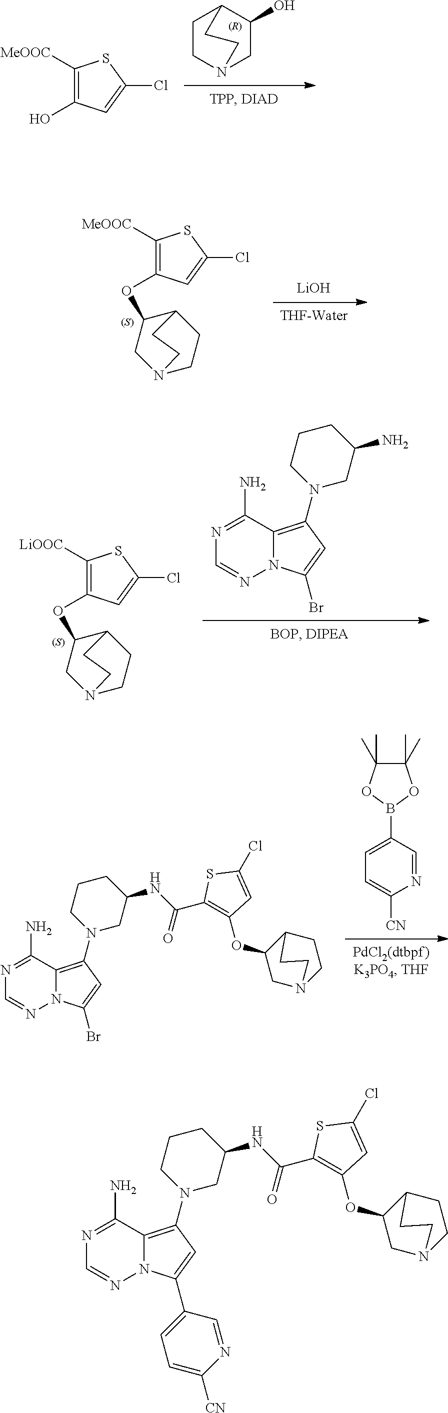

### Example 950A. Methyl (S)-5-chloro-3-(quinuclidin-3-yloxy)thiophene-2-carboxylate

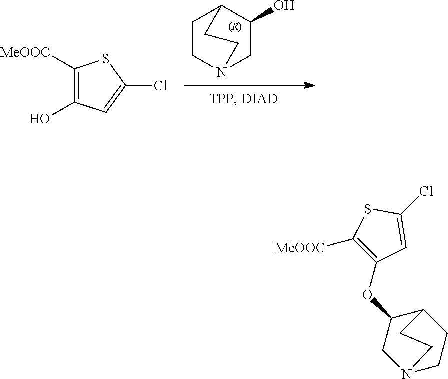

Analogous to the preparation of Example 946B, reaction of methyl 5-chloro-3-hydroxythiophene-2-carboxylate (600 mg, 3.12 mmol), (R)-quinuclidin-3-ol (396 mg, 3.12 mmol), triphenylphosphine (1.23 g, 4.67 mmol) and DIAD (0.666 mL, 3.43 mmol) afforded Example 950A (3.3 g, 29%) as a yellow oil. MS: [M+H]⁺=302.0.

### Example 950B. Lithium (S)-5-chloro-3-(quinuclidin-3-yloxy)thiophene-2-carboxylate

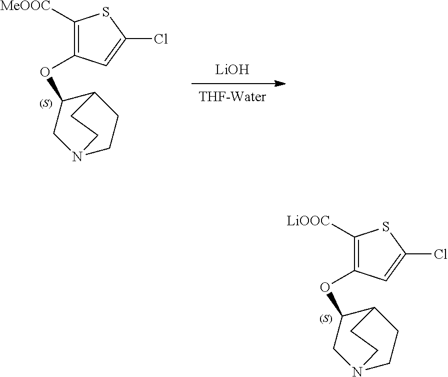

Analogous to the preparation of Example 946E, reaction of methyl (S)-5-chloro-3-(quinuclidin-3-yloxy)thiophene-2-carboxylate (900 mg, 2.98 mmol) and LiOH (143 mg, 5.96 mmol) afforded Example 950B (700 mg, 82%). MS: [M+H]⁺=288.2.

### Example 950C. N—((R)-1-(4-Amino-7-bromopyrrolo[2,1-f][1,2,4]triazin-5-yl)piperidin-3-yl)-5-chloro-3-(((S)-quinuclidin-3-yl)oxy)thiophene-2-carboxamide

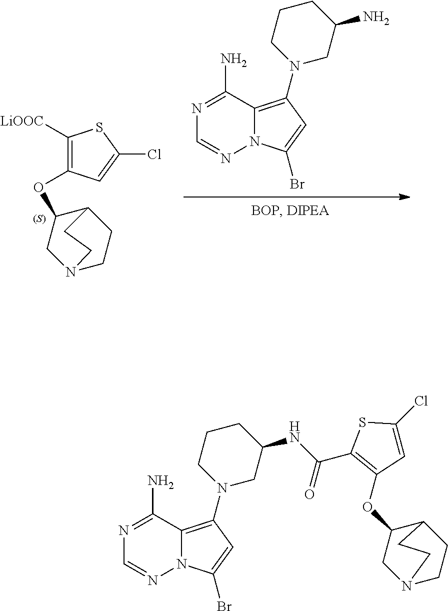

Analogous to the preparation of Example 946, reaction of Intermediate 61 (500 mg, 1.61 mmol), Example 950B (462 mg, 1.61 mmol), BOP (711 mg, 1.61 mmol) and DIEA (0.842 mL, 4.82 mmol) in DMF (10 mL) afforded Example 950C (400 mg, 43%). MS: [M+H]⁺=582.1.

### Example 950

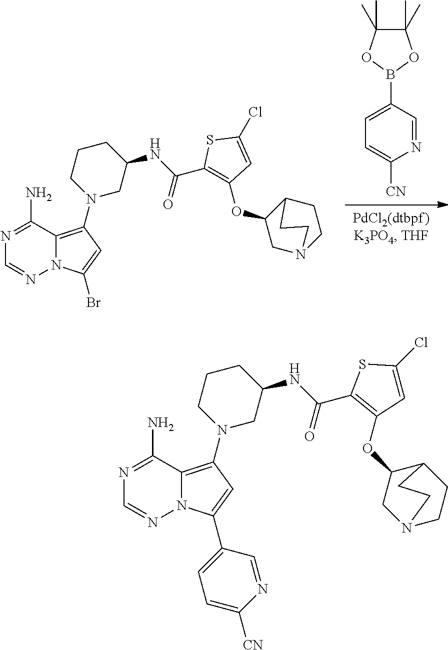

To a stirred solution of 5-(4,4,5,5-tetramethyl-1,3,2-dioxaborolan-2-yl)picolinonitrile (26.1 mg, 0.114 mmol), Example 950C (60 mg, 0.103 mmol) and 2M tripotassium phosphate (0.129 mL, 0.258 mmol) in THF (4 mL) was added 1,1′-bis(di-tert-butylphosphino)ferrocene dichloropalladium(II) (6.7 mg, 10.3 mmol) and the reaction mixture was heated at 70° C. for 5 h. The mixture was filtered, concentrated and purified by preparative HPLC to yield Example 950 (8.1 mg, 12.5%). LC-MS Method E: RT=1.58 min, [M+H]⁺=604.2; LC-MS Method F: RT=1.12 min, [M+H]⁺=604.2; ¹H NMR (400 MHz, DMSO-d₆) δ ppm 9.43 (s, 1H), 8.90-8.73 (m, 1H), 8.45-8.20 (m, 1H), 8.09 (d, J=8.5 Hz, 1H), 7.95 (s, 1H), 7.48-7.36 (m, 2H), 7.29 (s, 1H), 6.97-6.78 (m, 1H), 4.69 (br dd, J=2.1, 4.6 Hz, 1H), 4.29-4.15 (m, 1H), 3.09-2.98 (m, 1H), 2.89-2.75 (m, 5H), 2.26-2.20 (m, 1H), 2.15 (br d, J=3.5 Hz, 1H), 1.96-1.49 (m, 8H), 1.44-1.21 (m, 3H).

### Example 951

### Preparation of N—((R)-1-(4-Amino-7-(1-(2,2-difluoroethyl)-6-oxo-1,6-dihydropyridin-3-yl)pyrrolo[2,1-f][1,2,4]triazin-5-yl)piperidin-3-yl)-2-isopropyl-4-(((1R,3s,5S)-8-methyl-8-azabicyclo[3.2.1]octan-3-yl)oxy)thiazole-5-carboxamide

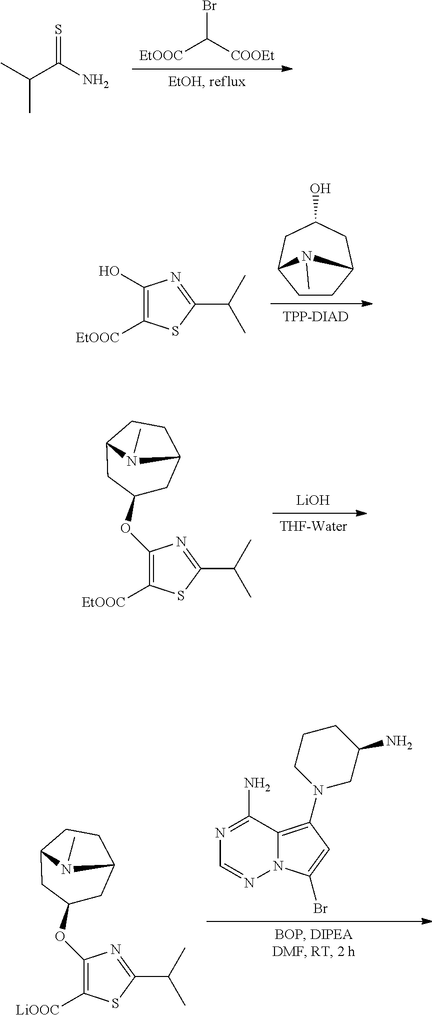

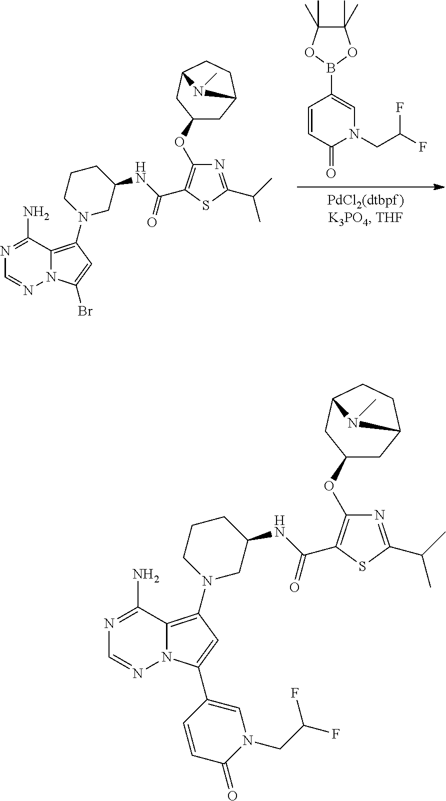 [SMILES: CC(C)c1nc(O[C@@H]2C[C@H]3CC[C@@H](C2)N3C)c(C(=O)N[C@@H]2CCCN(c3cc(Br)n4ncnc(N)c34)C2)s1 ; CC1(C)OB(c2ccc(=O)n(CC(F)F)c2)OC1(C)C ; "PdCl2(dtbpf)K3PO4, THF" ; CC(C)c1nc(O[C@@H]2C[C@H]3CC[C@@H](C2)N3C)c(C(=O)N[C@@H]2CCCN(c3cc(-c4ccc(=O)n(CC(F)F)c4)n4ncnc(N)c34)C2)s1]

### Example 951A. Ethyl 4-hydroxy-2-isopropylthiazole-5-carboxylate

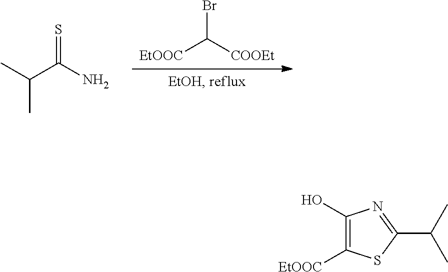

To a stirred solution of 2-methylpropanethioamide (5 g, 48.5 mmol) in ethanol (75 mL) was added diethyl 2-bromomalonate (8.27 mL, 48.5 mmol) and the reaction mixture was heated at 80° C. for 6 h. The mixture was cooled to RT and concentrated. Upon addition of water (100 mL), the precipitate formed was filtered and dried under vacuum to yield Example 951A (6.0 g, 57%). MS: [M+H]⁺=216.2.

### Example 951B. Ethyl 2-isopropyl-4-(((1R,3s,5S)-8-methyl-8-azabicyclo[3.2.1]octan-3-yl)oxy)thiazole-5-carboxylate

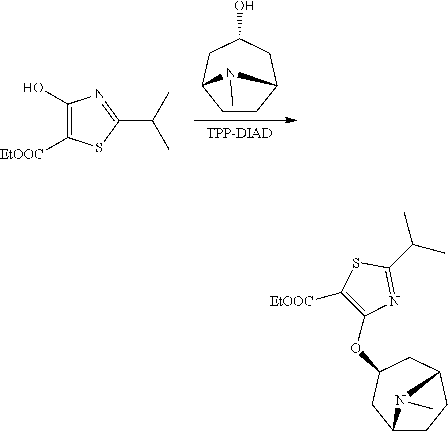

Analogous to the preparation of Example 946B, reaction of Example 951A (1.0 g, 4.65 mmol), (1R,3r,5S)-8-methyl-8-azabicyclo[3.2.1]octan-3-ol (0.656 g, 4.65 mmol), triphenylphosphine (1.83 g, 6.97 mmol) and DIAD (0.994 mL, 5.11 mmol) afforded Example 951B (1.2 g, 76%). MS: [M+H]⁺=339.1.

### Example 951C. Lithium 2-isopropyl-4-(((1R,3s,5S)-8-methyl-8-azabicyclo[3.2.1]octan-3-yl)oxy)thiazole-5-carboxylate

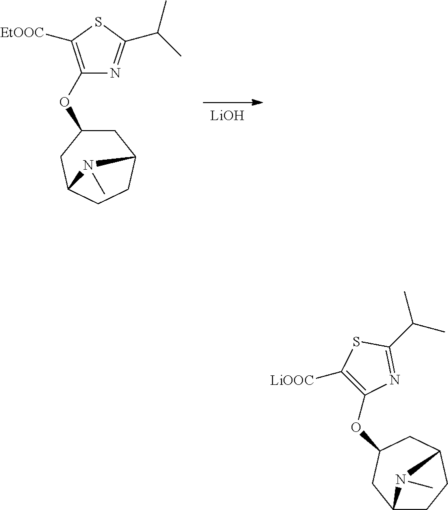

Analogous to the preparation of Example 946E, reaction of Example 951B (750 mg, 2.22 mmol) and LiOH (159 mg, 6.65 mmol) yielded Example 951C (600 mg, 87%). MS: [M+H]⁺=611.3.

### Example 951D. N—((R)-1-(4-amino-7-bromopyrrolo[2,1-f][1,2,4]triazin-5-yl)piperidin-3-yl)-2-isopropyl-4-(((1R,3s,5S)-8-methyl-8-azabicyclo[3.2.1]octan-3-yl)oxy)thiazole-5-carboxamide

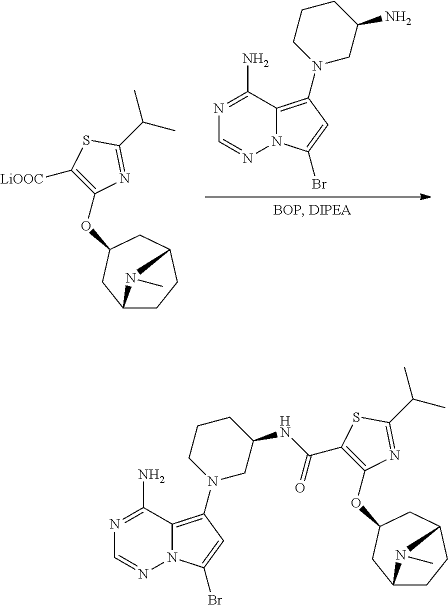

Analogous to the preparation of Example 1, reaction of Intermediate 61 (350 mg, 1.13 mmol), Example 951C (349 mg, 1.13 mmol), BOP (497 mg, 1.13 mmol) and DIEA (0.589 mL, 3.37 mmol) afforded Example 951D (550 mg, 81%). MS: [M+H]⁺=605.5.

### Example 951

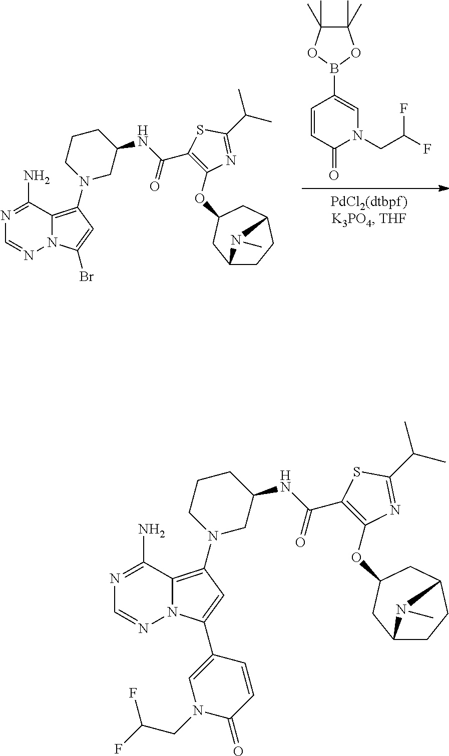 [SMILES: CC(C)c1nc(O[C@@H]2C[C@H]3CC[C@@H](C2)N3C)c(C(=O)N[C@@H]2CCCN(c3cc(Br)n4ncnc(N)c34)C2)s1 ; CC1(C)OB(c2ccc(=O)n(CC(F)F)c2)OC1(C)C ; "PdCl2(dtbpf)K3PO4, THF" ; CC(C)c1nc(O[C@@H]2C[C@H]3CC[C@@H](C2)N3C)c(C(=O)N[C@@H]2CCCN(c3cc(-c4ccc(=O)n(CC(F)F)c4)n4ncnc(N)c34)C2)s1]

Analogous to the preparation of Example 950, reaction of Example 951D (50 mg, 0.083 mmol), 1-(2,2-difluoroethyl)-5-(4,4,5,5-tetramethyl-1,3,2-dioxaborolan-2-yl)pyridin-2(1H)-one (30.7 mg, 0.108 mmol), 1,1′-bis(di-tert-butylphosphino)ferrocene dichloropalladium(II) (5.4 mg, 8.3 mmol) and 2M tripotassium phosphate (0.124 mL, 0.249 mmol) yielded Example 951 (9.9 mg, 17%). LC-MS Method E: RT=1.91 min, [M+H]⁺=682.3; LC-MS Method F: RT=1.03 min, [M+H]⁺=682.3; ¹H NMR (400 MHz, DMSO-d₆) δ ppm 8.50 (d, J=2.3 Hz, 1H), 8.11 (dd, J=2.5, 9.5 Hz, 1H), 7.83 (s, 1H), 7.48-7.36 (m, 2H), 7.32 (br s, 1H), 6.94 (s, 1H), 6.58 (d, J=9.6 Hz, 1H), 6.51-6.17 (m, 1H), 5.24-5.08 (m, 1H), 4.57-4.37 (m, 2H), 4.20 (br d, J=2.9 Hz, 1H), 3.23-3.09 (m, 3H), 2.94 (br s, 2H), 2.31-1.96 (m, 4H), 1.90-1.47 (m, 7H), 1.30 (d, J=6.8 Hz, 8H).

### Example 952

### Preparation of N—((R)-1-(7-(1-Acetyl-2,5-dihydro-1H-pyrrol-3-yl)-4-aminopyrrolo[2,1-f][1,2,4]triazin-5-yl)piperidin-3-yl)-5-chloro-3-((6-methyl-6-azabicyclo[3.2.1]octan-3-yl)oxy)thiophene-2-carboxamide

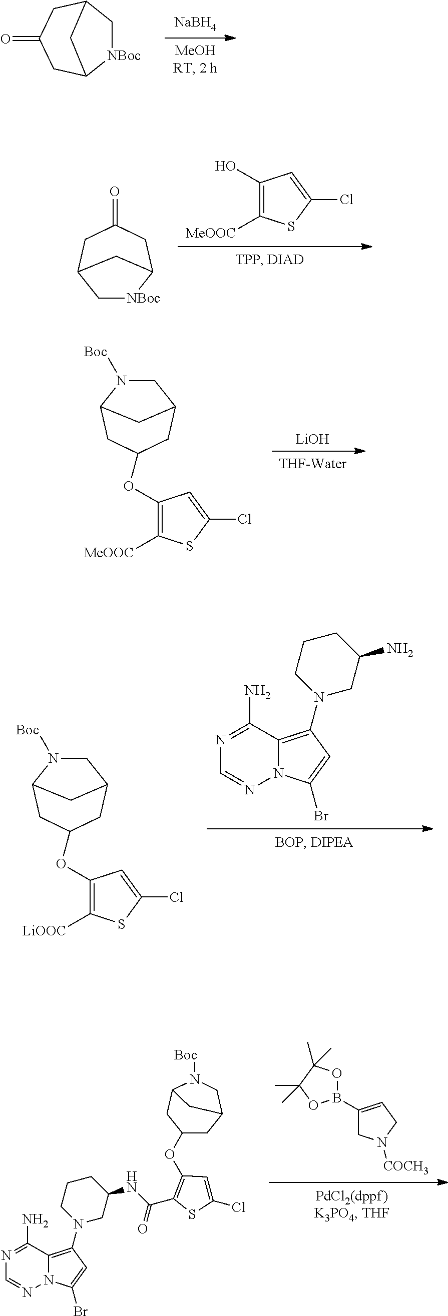

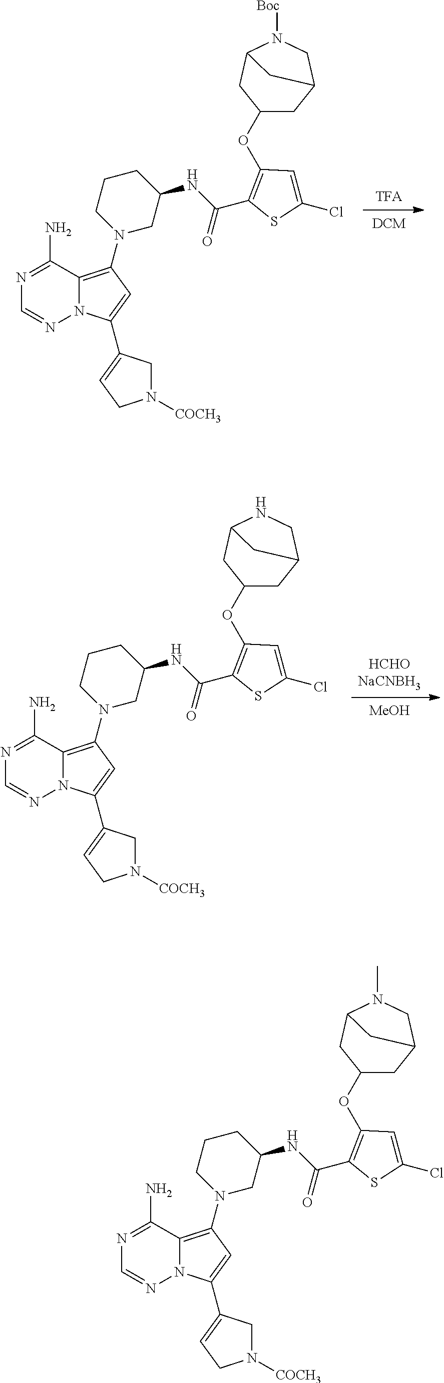

### Example 952A. tert-Butyl 3-hydroxy-6-azabicyclo[3.2.1]octane-6-carboxylate

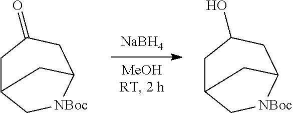 [SMILES: CC(C)(C)OC(=O)N1CC2CC(=O)CC1C2 ; "MeOHRT, 2 h" ; "NaBH4" ; CC(C)(C)OC(=O)N1CC2CC(O)CC1C2]

To a stirred solution of tert-butyl 3-oxo-6-azabicyclo[3.2.1]octane-6-carboxylate (1.0 g, 4.44 mmol) in MeOH (10 mL) at 0° C. was added NaBH₄ (0.202 g, 5.33 mmol) and the reaction mixture was stirred at 0° C. for 1 h. Mixture was concentrated and partitioned between EtOAc and saturated NH₄Cl solution. The organic phase was dried over Na₂SO₄, filtered and concentrated to yield Example 952A (0.7 g, 69%) as a colorless liquid.

### Example 952B. tert-Butyl 3-((5-chloro-2-(methoxycarbonyl)thiophen-3-yl)oxy)-6-azabicyclo[3.2.1]octane-6-carboxylate

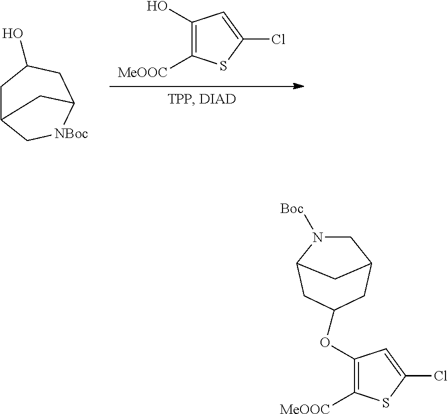

Analogous to the preparation of Example 946B, reaction of methyl 5-chloro-3-hydroxythiophene-2-carboxylate (0.7 g, 3.63 mmol), Example 952A (0.826 g, 3.63 mmol), triphenylphosphine (1.24 g, 4.72 mmol) and DIAD (0.707 mL, 3.63 mmol) afforded Example 952B (0.6 g, 41%) as a colorless liquid. MS: [M+H]⁺=402.15.

### Example 952C. Lithium 3-((6-(tert-butoxycarbonyl)-6-azabicyclo[3.2.1]octan-3-yl)oxy)-5-chlorothiophene-2-carboxylate

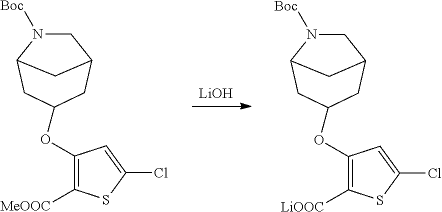

Analogous to the preparation of Example 946E, reaction of Example 952B (0. g, 1.49 mmol) and lithium hydroxide monohydrate (2M solution) (2.24 mL, 4.48 mmol) afforded Example 952C (0.7 g, 69%) as an off-white solid. MS: [M+H]⁺=386.3.

### Example 952D. tert-Butyl 3-((2-(((R)-1-(4-amino-7-bromopyrrolo[2,1-f][1,2,4]triazin-5-yl)piperidin-3-yl)carbamoyl)-5-chlorothiophen-3-yl)oxy)-6-azabicyclo[3.2.1]octane-6-carboxylate

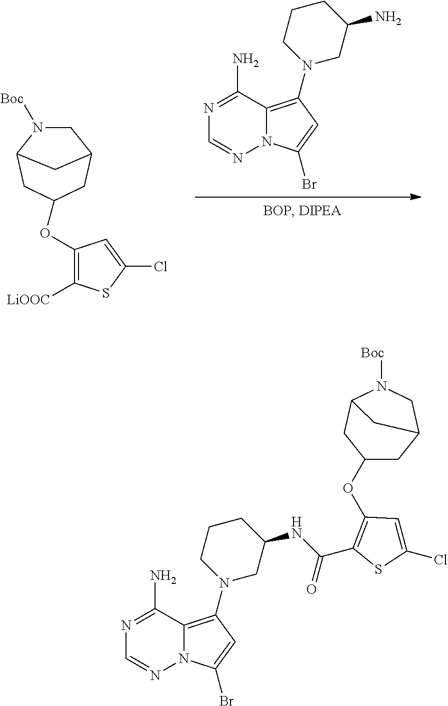

Analogous to the preparation of Example 946, reaction of Intermediate 61, Example 952C (0.409 g, 1.04 mmol), BOP (0.560 g, 1.27 mmol) and DIEA (0.603 mL, 3.45 mmol) afforded crude product, which was purified by silica gel chromatography (eluting with 0-5% MeOH in DCM). The diastereomers were separated by Preparative SFC [Preparative SFC Conditions: Whelk-01(R,R) (250*4.6 mm, 5u): % CO₂: 50%; % Cosolvent: 50% (5 mM NH₄OAc in MeOH+MeCN (1:1)); Total Flow: 4 mL/min; Back Pressure: 100 bars; Temperature: 35° C.; Detection: UV at 250 nm] to yield First eluting isomer (Iso-1): 150 mg, 19%, as a yellow gummy solid; SFC Method: RT=2.93 min; 100% ee; MS: [M+H]⁺=682.15 and Second eluting isomer (Iso-2): 150 mg, 19% as a yellow gummy solid: SFC Method: RT=3.99 min; 99% ee; MS: [M+H]⁺=682.15. The first eluting isomer (Iso-1) was taken to next step.

### Example 952E. tert-Butyl 3-((2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)-6-azabicyclo[3.2.1]octane-6-carboxylate

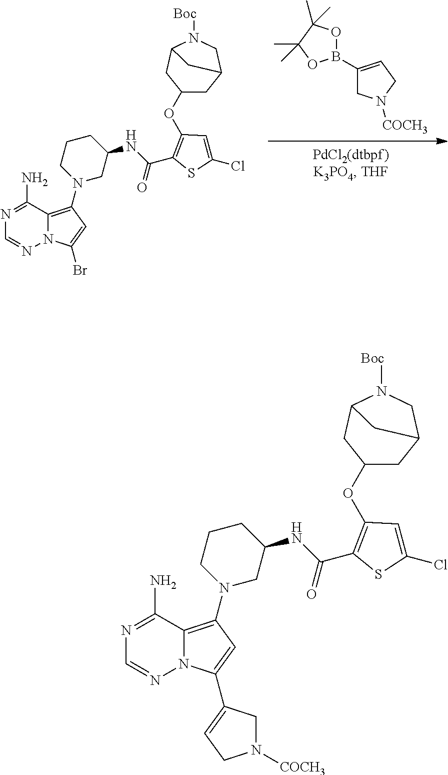 [SMILES: CC(C)(C)OC(=O)N1CC2CC(Oc3cc(Cl)sc3C(=O)N[C@@H]3CCCN(c4cc(Br)n5ncnc(N)c45)C3)CC1C2 ; CC(=O)N1CC=C(B2OC(C)(C)C(C)(C)O2)C1 ; "PdCl2(dtbpf)K3PO4, THF" ; CC(=O)N1CC=C(c2cc(N3CCC[C@@H](NC(=O)c4sc(Cl)cc4OC4CC5CC(C4)N(C(=O)OC(C)(C)C)C5)C3)c3c(N)ncnn23)C1]

Analogous to the preparation of Example 947, reaction of Example 952D (120 mg, 0.176 mmol), 1-(3-(4,4,5,5-tetramethyl-1,3,2-dioxaborolan-2-yl)-2,5-dihydro-1H-pyrrol1-yl)ethan-1-one (66.8 mg, 0.282 mmol), 1,1′-bis(di-tert-butylphosphino)ferrocene dichloropalladium(II) (8.0 mg, 0.012 mmol) and potassium phosphate tribasic (74.8 mg, 0.352 mmol) afforded Example 952E (100 mg, 80%) as a yellow gummy solid. MS: [M+H]⁺=711.5.

### Example 952F. 3-((6-Azabicyclo[3.2.1]octan-3-yl)oxy)-N—((R)-1-(7-(1-acetyl-2,5-dihydro-1M-pyrrol-3-yl)-4-aminopyrrolo[2,1-f][1,2,4]triazin-5-yl)piperidin-3-yl)-5-chlorothiophene-2-carboxamide

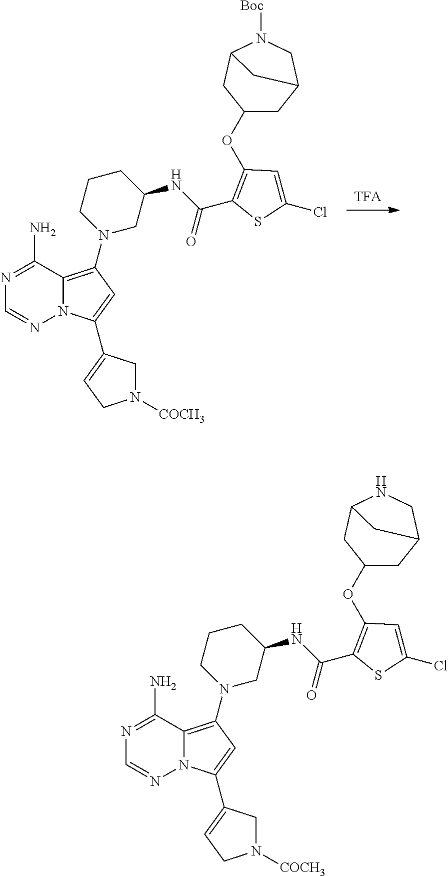 [SMILES: CC(=O)N1CC=C(c2cc(N3CCC[C@@H](NC(=O)c4sc(Cl)cc4OC4CC5CC(C4)N(C(=O)OC(C)(C)C)C5)C3)c3c(N)ncnn23)C1 ; CC(=O)N1CC=C(c2cc(N3CCC[C@@H](NC(=O)c4sc(Cl)cc4OC4CC5CNC(C5)C4)C3)c3c(N)ncnn23)C1 ; "TFA"]

Analogous to the preparation of Example 946C, reaction of Example 952E (30 mg, 0.042 mmol) with TFA afforded Example 952F as a brown gummy solid. MS: [M+H]⁺=612.3.

### Example 952

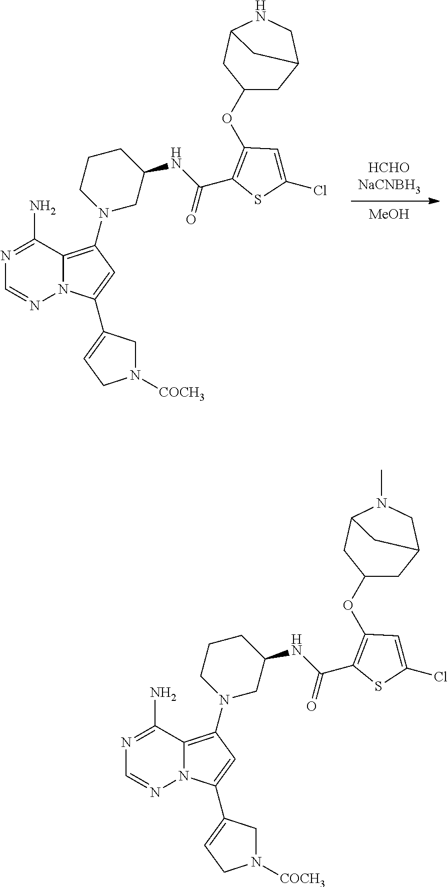 [SMILES: "MeOH" ; CC(=O)N1CC=C(c2cc(N3CCC[C@@H](NC(=O)c4sc(Cl)cc4OC4CC5CNC(C5)C4)C3)c3c(N)ncnn23)C1 ; "HCHONaCNBH3" ; CC(=O)N1CC=C(c2cc(N3CCC[C@@H](NC(=O)c4sc(Cl)cc4OC4CC5CC(C4)N(C)C5)C3)c3c(N)ncnn23)C1]

To a stirred solution of Example 952F (40 mg, 0.055 mmol), formaldehyde (38% solution, 7.6 μl, 0.276 mmol) and acetic acid (0.3 μl, 5.5 mmol) in methanol (2 mL) was added sodium cyanoborohydride (6.9 mg, 0.11 mmol) and the reaction mixture was stirred at rt for 2 h. The mixture was filtered, concentrated and purified by preparative HPLC to yield Example 952 (17.9 mg, 52%). LC-MS Method F: RT=1.21 min, [M+H]⁺=625.3; LC-MS Method E: RT=1.32 min, [M+H]⁺=625.2; ¹H NMR (400 MHz, DMSO-d₆) δ ppm 9.38 (br s, 1H), 7.90 (s, 1H), 7.85-7.75 (m, 1H), 7.34 (s, 1H), 6.78 (td, J=1.7, 7.9 Hz, 1H), 6.73 (d, J=14.3 Hz, 1H), 4.82-4.65 (m, 2H), 4.53-4.39 (m, 2H), 4.30-4.09 (m, 2H), 3.96-3.75 (m, 4H), 3.21-3.18 (m, 2H), 3.14-3.08 (m, 2H), 3.00 (br s, 2H), 2.86 (d, J=5.3 Hz, 3H), 2.77 (s, 2H), 2.65 (br d, J=4.8 Hz, 3H), 2.32-2.23 (m, 3H), 2.14-2.00 (m, 6H), 1.95 (br s, 2H), 1.83 (br dd, J=1.1, 12.9 Hz, 3H), 1.56 (br s, 1H).

### Example 953

### Methyl 3-(4-amino-5-((3R)-3-(4-((6-isopropyl-6-azabicyclo[3.2.1]octan-3-yl)oxy)-2-methylthiazole-5-carboxamido)piperidin-1-yl)pyrrolo[2,1-f][1,2,4]triazin-7-yl)-2,5-dihydro-1H-pyrrole-1-carboxylate

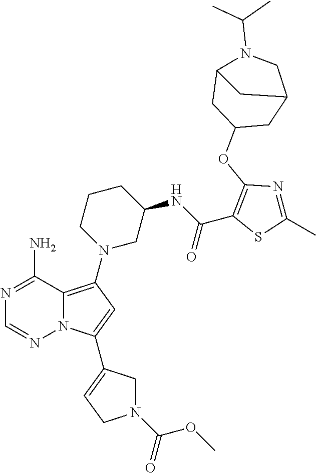 [SMILES: COC(=O)N1CC=C(c2cc(N3CCC[C@@H](NC(=O)c4sc(C)nc4OC4CC5CC(C4)N(C(C)C)C5)C3)c3c(N)ncnn23)C1]

Example 953 was prepared using the same procedure as shown in Example 952. Analogous to the synthesis of Example 952B, intermediate 2 was synthesized and was separated by silica-gel column chromatography to obtain diastereomeric mixture-1 & diastereomeric mixture-2. Diasteromeric mixture-1 afforded Iso-1 and Iso-2, whereas diastereomeric mixture-2 afforded Iso-3 and Iso-4 after SFC purification. Purification details are described below. The second eluting isomer (Iso-2) was taken to next step to make Example 953.

### Intermediate 2. Ethyl 4-((6-(tert-butoxycarbonyl)-6-azabicyclo[3.2.1]octan-3-yl)oxy)-2-methylthiazole-5-carboxylate

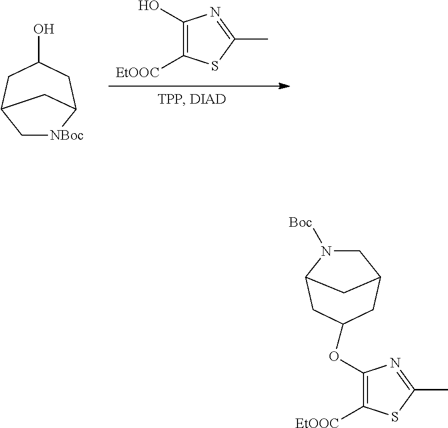

Diastereomeric mixture-1 was purified by SFC [Preparative SFC Conditions: Chiralpak IC (250*4.6 mm; 5u); % CO₂: 85%; % Cosolvent: 15% (5 mM NH₄OAc in MeOH+MeCN(1:1)); Total Flow: 3 mL/min; Back Pressure: 100 bars: Temperature: 35° C.; Detection: UV at 250 nm] to get two chiral isomers (Iso-1 and Iso-2). First eluting isomer (Iso-1): 600 mg, 19%, yellow thick liquid: SFC Method: RT=3.42 min; 99.6% cc; MS: [M+H]⁺=397.2. Second eluting Isomer (Iso-2): 600 mg, 19%, yellow thick liquid: SFC Method: RT=3.82 min; 99.5% cc; MS: [M+H]⁺=397.2.

Diastereomeric mixture-2 was purified by SFC [Preparative SFC Conditions: Whelk-01(R,R)(250*4.6 mm, 5u); % CO₂: 50%; % Cosolvent: 50% (5 mM NH₄OAc in MeOH+MeCN(1:1)); Total Flow: 4 mL/min; Back Pressure: 100 bars; Temperature: 35° C.; Detection: UV at 250 nm] to get two chiral isomers (Iso-3 and Iso-4). First eluting isomer (Iso-3): 500 mg, 15.7%, yellow thick liquid. SFC Method: RT=2.97 min; 99.8% cc: MS: [M+H]⁺=397.2. Second eluting Isomer (Iso-4): 400 mg, 12.6%, yellow thick liquid, SFC Method: RT=3.33 min; 98.7% ee: MS: [M+H]⁺=397.2.

Example 953. LC-MS Method E: RT=1.45 min, [M+H]⁺=650.3; LC-MS Method F: RT=0.91 min, [M+H]⁺=650.3; ¹H NMR (400 MHz, DMSO-d₆) δ ppm 7.85 (s, 1H), 7.31 (br s, 1H), 6.76-6.67 (m, 2H), 5.21-5.12 (m, 1H), 4.53 (ddd, J=1.8, 2.7, 13.3 Hz, 2H), 4.34-4.24 (m, 2H), 4.10 (br s, 1H), 3.65 (d, J=4.3 Hz, 3H), 3.09 (br s, 1H), 2.89 (br s, 5H), 2.61 (s, 3H), 2.33 (td, J=1.9, 3.6 Hz, 2H), 2.19 (br dd, J=2.1, 3.9 Hz, 1H), 1.87-1.71 (m, 3H), 1.49 (br s, 4H), 1.08-0.90 (m, 6H).

### Example 954

### Preparation of 4-((1-oxa-8-azaspiro[4.5]decan-3-yl)oxy)-N—((R)-1-(4-amino-7-(4-carbamoyl-3-fluorophenyl)pyrrolo[2,1-f][1,2,4]triazin-5-yl)piperidin-3-yl)-2-methylthiazole-5-carboxamide

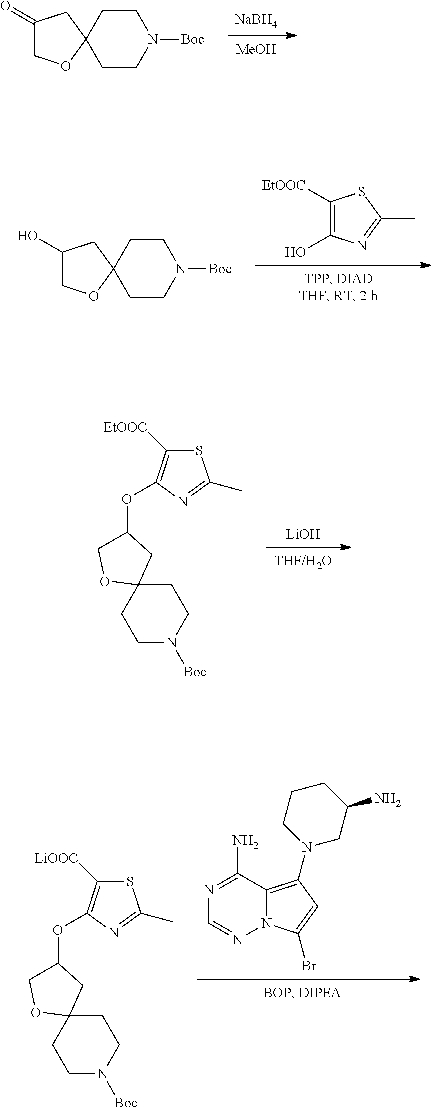

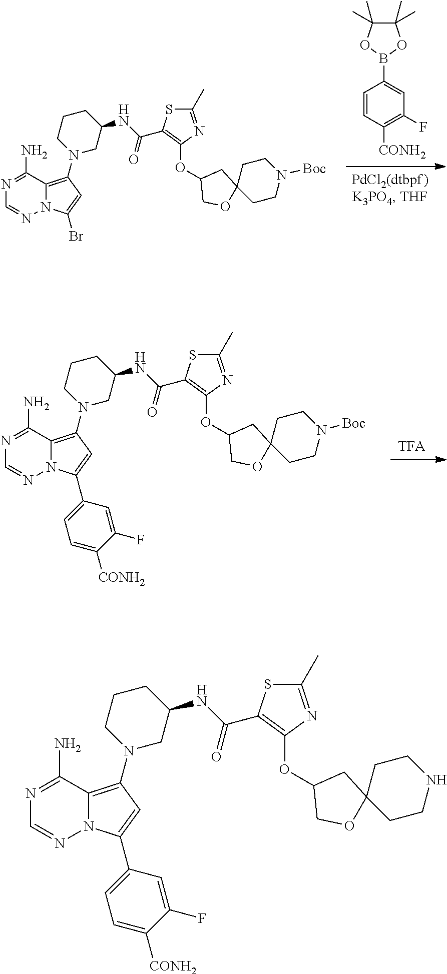 [SMILES: Cc1nc(OC2COC3(CCN(C(=O)OC(C)(C)C)CC3)C2)c(C(=O)N[C@@H]2CCCN(c3cc(Br)n4ncnc(N)c34)C2)s1 ; CC1(C)OB(c2ccc(C(N)=O)c(F)c2)OC1(C)C ; "PdCl2(dtbpf)K3PO4, THF" ; Cc1nc(OC2COC3(CCN(C(=O)OC(C)(C)C)CC3)C2)c(C(=O)N[C@@H]2CCCN(c3cc(-c4ccc(C(N)=O)c(F)c4)n4ncnc(N)c34)C2)s1 ; "TFA" ; Cc1nc(OC2COC3(CCNCC3)C2)c(C(=O)N[C@@H]2CCCN(c3cc(-c4ccc(C(N)=O)c(F)c4)n4ncnc(N)c34)C2)s1]

Example 954A. tert-Butyl 3-hydroxy-1-oxa-8-azaspiro[4.5]decane-8-carboxylate

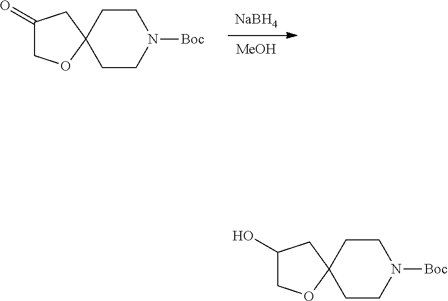 [SMILES: "MeOH" ; CC(C)(C)OC(=O)N1CCC2(CC1)CC(=O)CO2 ; "NaBH4" ; CC(C)(C)OC(=O)N1CCC2(CC1)CC(O)CO2]

Analogous to the preparation of Example 946A, reaction of tert-butyl 3-oxo-1-oxa-8-azaspiro[4.5]decane-8-carboxylate (2 g, 7.83 mmol) and NaBH₄ (0.593 g, 15.7 mmol) yielded Example 954A (2 g, 99%) as colorless oil; ¹H NMR (300 MHz, DMSO-d6) ppm 1.38 (s, 10H) 1.77-1.50 (m, 3H) 1.95-1.75 (m, 1H) 3.64-3.11 (m, 5H) 3.31-3.11 (m, 2H) 3.30-3.14 (m, 1H) 3.50-3.30 (m, 3H) 3.60-3.53 (m, 1H) 3.89-3.72 (m, 1H) 4.37-4.21 (m, 1H) 4.96-4.80 (m, 1H).

### Example 954B. tert-Butyl 3-((5-(ethoxycarbonyl)-2-methylthiazol-4-yl)oxy)-1-oxa-8-azaspiro[4.5]decane-8-carboxylate

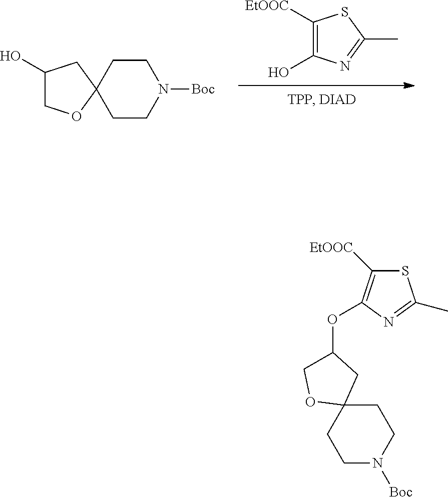

Analogous to the preparation of Example 1B, reaction of tert-butyl 3-hydroxy-1-oxa-8-azaspiro[4.5]decane-8-carboxylate (1.9 g, 7.38 mmol), ethyl 4-hydroxy-2-methylthiazole-5-carboxylate (1.38 g, 7.38 mmol), triphenylphosphine (3.29 g, 12.6 mmol) and DIAD (1.94 g, 9.60 mmol) afforded crude product, which was purified by silica-gel chromatography (0% to 20% EtOAc/hexanes gradient). The racemate was resolved by preparative SFC [Conditions: Column/dimensions: Chiralpak IG (250×30 mm, 5u); % CO₂: 70%, % Cosolvent: 30% of 5 mM Ammonium acetate in MeCN/MeOH (1:1); Total Flow: 130 g/min; Back Pressure: 100 bar; Temperature: 30° C.; UV: 240 nm] to yield First eluting isomer (Iso-1): (1 g, 32% as a yellow syrup), SFC Method: RT=2.20 min; 100% ee; MS: [M−Boc+2H]⁺=327.2 and Second eluting isomer (Iso-2): (1 g, 32% as a yellow syrup), SFC Method: RT=3.01 min; 99% ee: MS: [M−Boc+2H]⁺=327.2. The first eluting isomer (Iso-1) was taken to the next step as Example 954B.

### Example 954C. Lithium 4-((8-(tert-butoxycarbonyl)-1-oxa-8-azaspiro[4.5]decan-3-yl)oxy)-2-methylthiazole-5-carboxylate

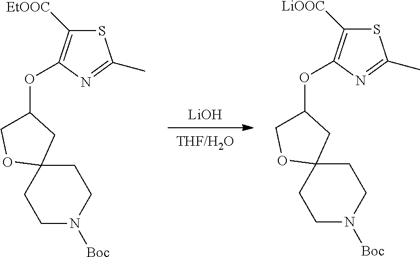

Analogous to the preparation of Example 946E, reaction of Example 954B (1.0 g, 2.35 mmol) and lithium hydroxide (0.140 g, 5.86 mmol) yielded Example 954C (900 mg, 96%) as an off-white solid. MS: [M+H]⁺=299.2.

### Example 954D. tert-Butyl 3-((5-(((R)-1-(4-amino-7-bromopyrrolo[2,1-f][1,2,4]triazin-5-yl)piperidin-3-yl)carbamoyl)-2-methylthiazol-4-yl)oxy)-1-oxa-8-azaspiro[4.5]decane-8-carboxylate

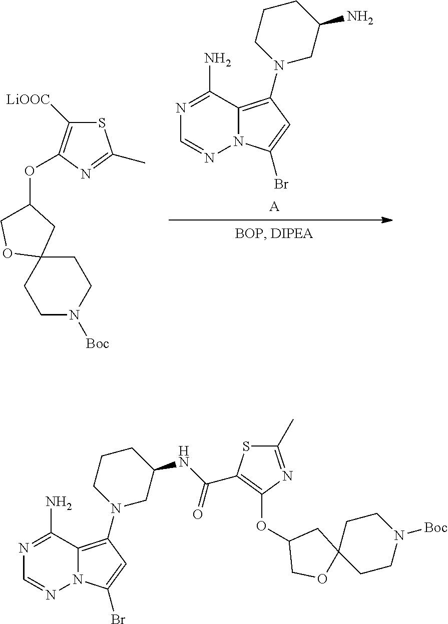

Analogous to the preparation of Example 946, reaction of Example 954C (900 mg, 2.26 mmol), Intermediate 61 (703 mg, 2.26 mmol), BOP (999 mg, 2.26 mmol) and DIEA (1.18 mL, 6.78 mmol) yielded Example 954D (1.5 g, 96%) as a brown paste. MS: [M+H]⁺=691.5.

### Example 954E. tert-Butyl 3-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)-1-oxa-8-azaspiro[4.5]decane-8-carboxylate

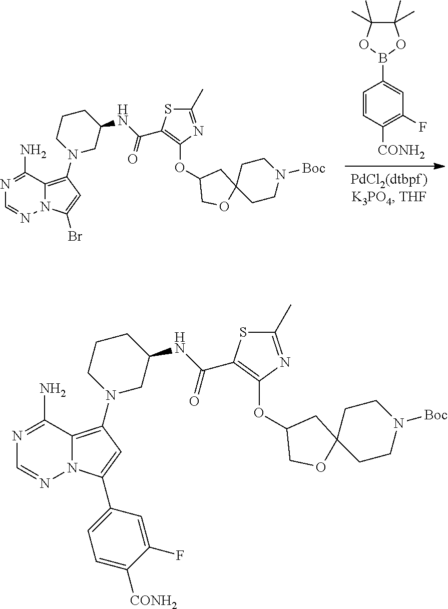 [SMILES: Cc1nc(OC2COC3(CCN(C(=O)OC(C)(C)C)CC3)C2)c(C(=O)N[C@@H]2CCCN(c3cc(Br)n4ncnc(N)c34)C2)s1 ; CC1(C)OB(c2ccc(C(N)=O)c(F)c2)OC1(C)C ; "PdCl2(dtbpf)K3PO4, THF" ; Cc1nc(OC2COC3(CCN(C(=O)OC(C)(C)C)CC3)C2)c(C(=O)N[C@@H]2CCCN(c3cc(-c4ccc(C(N)=O)c(F)c4)n4ncnc(N)c34)C2)s1]

Analogous to the preparation of Example 947, reaction of Example 954D (50 mg, 0.072 mmol), 2-fluoro-4-(4,4,5,5-tetramethyl-1,3,2-dioxaborolan-2-yl)benzamide (38.3 mg, 0.145 mmol), 1,1′-Bis(di-tert-butylphosphino)ferrocene]dichloropalladium(II)(2.4 mg, 3.6 mmol) and 2M tripotassium phosphate (0.108 mL, 0.217 mmol) afforded Example 954E (50 mg, 92%) as a brown oil. MS: [M+H]⁺=750.6.

### Example 954

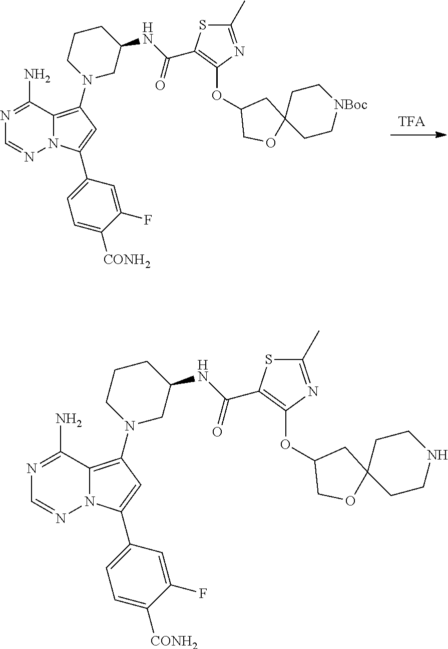 [SMILES: Cc1nc(OC2COC3(CCN(C(=O)OC(C)(C)C)CC3)C2)c(C(=O)N[C@@H]2CCCN(c3cc(-c4ccc(C(N)=O)c(F)c4)n4ncnc(N)c34)C2)s1 ; "TFA" ; Cc1nc(OC2COC3(CCNCC3)C2)c(C(=O)N[C@@H]2CCCN(c3cc(-c4ccc(C(N)=O)c(F)c4)n4ncnc(N)c34)C2)s1]

Analogous to the preparation of Example 946C, Example 954E (50 mg, 0.067 mmol) and TFA afforded Example 954 (17.5 mg, 36%). LC-MS Method E: RT=1.09 min, [M+H]⁺=650.3; LC-MS Method F: RT=0.858 min, [M+H]⁺=650.3; ¹H NMR (400 MHz, DMSO-d₆) δ ppm 8.33 (s, 2H), 8.13 (dd, J=1.4, 12.6 Hz, 1H), 8.00 (dd, J=1.8, 8.3 Hz, 1H), 7.93 (s, 1H), 7.78-7.63 (m, 7H), 7.25 (s, 1H), 5.59-5.53 (m, 1H), 4.20 (s, 1H), 4.09-4.03 (m, 2H), 3.03 (br s, 4H), 2.62 (s, 4H), 2.30-2.22 (m, 1H), 1.84 (br s, 8H).

### Example 955

### (R)—N-(1-(4-amino-7-(6-cyanopyridin-3-yl)pyrrolo[2,1-f][1,2,4]triazin-5-yl)piperidin-3-yl)-2-methyl-4-((7-methyl-7-azaspiro[3.5]nonan-2-yl)oxy)thiazole-5-carboxamide

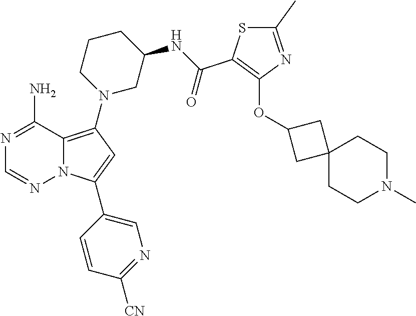 [SMILES: Cc1nc(OC2CC3(CCN(C)CC3)C2)c(C(=O)N[C@@H]2CCCN(c3cc(-c4ccc(C#N)nc4)n4ncnc(N)c34)C2)s1]

Example 955 was prepared using the same procedure as shown in Example 954.

LC-MS Method E: RT=1.36 min, [M+H]⁺=613.3; LC-MS Method F: RT=0.95 min, [M+H]⁺=613.3; ¹H NMR (400 MHz, DMSO-d₆) δ ppm 9.44 (d, J=1.5 Hz, 1H), 8.82 (dd, J=2.4, 8.4 Hz, 1H), 8.10 (d, J=8.3 Hz, 1H), 7.97 (s, 1H), 7.42 (s, 1H), 5.31-5.14 (m, 1H), 4.25-4.10 (m, 1H), 3.21-3.14 (m, 1H), 3.07-2.90 (m, 2H), 2.60 (s, 3H), 2.32-2.25 (m, 2H), 2.02 (br s, 3H), 1.91 (s, 1H), 1.88-1.76 (m, 5H), 1.72-1.60 (m, 1H), 1.50 (br t, J=5.5 Hz, 2H), 1.46-1.38 (m, 2H)

### Example 956

### Preparation of N—((R)-1-(4-Amino-7-(5-carbamoyl-6-(2,2-difluoroethoxy)pyridin-2-yl)pyrrolo[2,1-f][1,2,4]triazin-5-yl)piperidin-3-yl)-4-(((1R,3s,5S)-8-isopropyl-8-azabicyclo[3.2.1]octan-3-yl)oxy)-2-methylthiazole-5-carboxamide

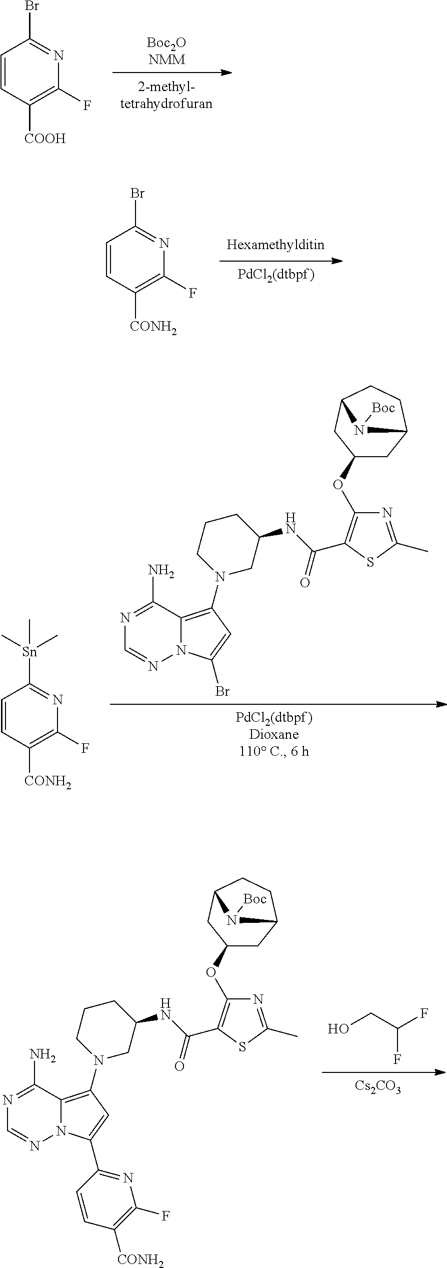

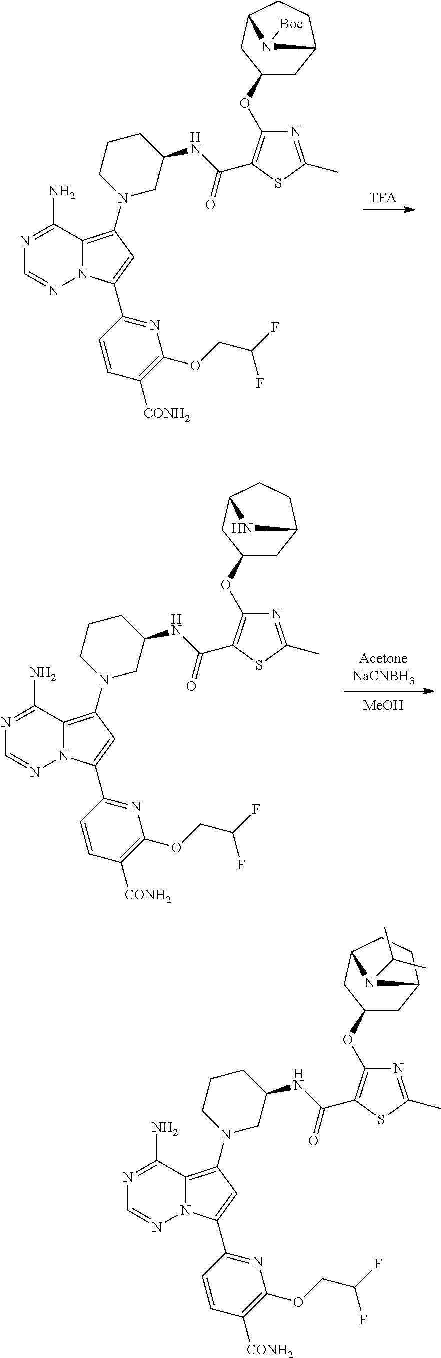

### Example 956A. 6-Bromo-2-fluoronicotinic acid

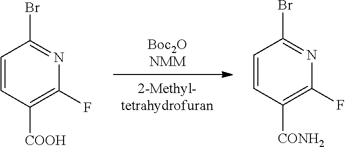

To a stirred solution of 6-bromo-2-fluoronicotinic acid (2.5 g, 11.4 mmol), N-methylmorpholine (1.62 mL, 14.8 mmol) and Boc₂ (3.69 mL, 15.9 mmol) in 2-methyltetrahydrofuran (25 mL, 11.4 mmol) was added ammonium bicarbonate (1.71 g, 21.6 mmol) portion wise and mixture was stirred at RT for 16 h. The reaction mixture was evaporated and partitioned between EtOAc and saturated NH₄Cl solution. The organic phase was dried over Na₂SO₄, filtered and concentrated to yield Example 956A (2 g, 80%) as a yellow solid. MS: [M−H]⁺=219.0.

### Example 956B. 2-Fluoro-6-(trimethylstannyl)nicotinamide

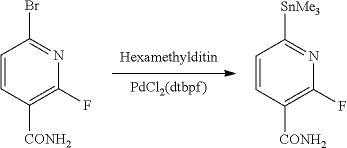

To a mixture 6-bromo-2-fluoronicotinamide (1 g, 4.57 mmol) in 1,2-dimethoxyethane (5 mL) and hexamethylditin (1.04 mL, 5.02 mmol in 1,4-dioxane (2 mL) was added 1,1′-bis(di-tert-butylphosphino)ferrocene dichloropalladium(II) (0.298 g, 0.457 mmol) and the reaction mixture was heated at 85° C. for 12 h. It was concentrated in vacuum to yield Example 956B (1.2 g, 87%) as a brown gummy oil and taken up for the next step as such. MS: [M+H]⁺=305.0.

### Example 956C. tert-Butyl (1R,3s,5S)-3-((5-(((R)-1-(4-amino-7-(5-carbamoyl-6-fluoropyridin-2-yl)pyrrolo[2,1-f][1,2,4]triazin-5-yl)piperidin-3-yl)carbamoyl)-2-methylthiazol-4-yl)oxy)-8-azabicyclo[3.2.1]octane-8-carboxylate

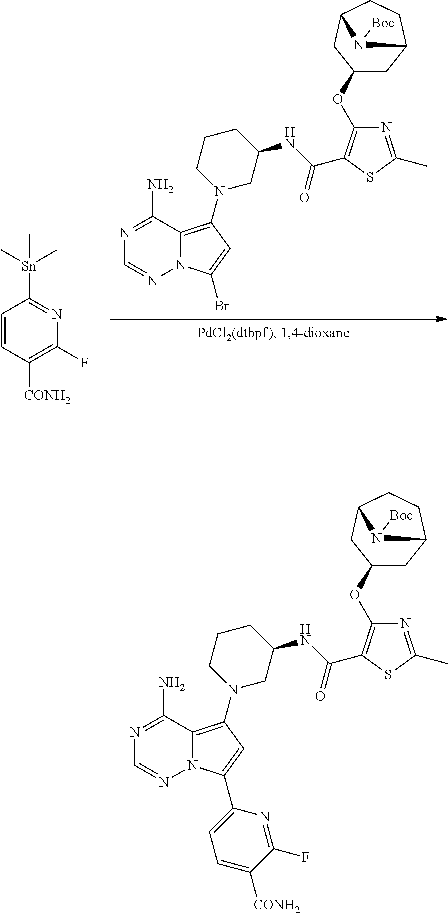 [SMILES: [CH3][Sn]([CH3])([CH3])[c]1ccc(C(N)=O)c(F)n1 ; Cc1nc(O[C@@H]2C[C@H]3CC[C@@H](C2)N3C(=O)OC(C)(C)C)c(C(=O)N[C@@H]2CCCN(c3cc(Br)n4ncnc(N)c34)C2)s1 ; "PdCl2(dtbpf), 1,4-dioxane" ; Cc1nc(O[C@@H]2C[C@H]3CC[C@@H](C2)N3C(=O)OC(C)(C)C)c(C(=O)N[C@@H]2CCCN(c3cc(-c4ccc(C(N)=O)c(F)n4)n4ncnc(N)c34)C2)s1]

To a mixture of tert-butyl (1R,3s,5S)-3-((5-(((R)-1-(4-amino-7-bromopyrrolo[2,1-f][1,2,4]triazin-5-yl)piperidin-3-yl)carbamoyl)-2-methylthiazol-4-yl)oxy)-8-azabicyclo[3.2.1]octane-8-carboxylate (800 mg, 1.21 mmol) and 2-fluoro-6-(trimethylstannyl)nicotinamide (733 mg, 2.42 mmol) in 1,4-dioxane (15 mL) was added 1,1′-bis(di-tert-butylphosphino)ferrocene dichloropalladium(II) (79 mg, 0.121 mmol) and the reaction mixture was heated to 85° C. for 12 h. The reaction mixture was concentrated in vacuo and residue obtained was purified by flash chromatography (0% to 10% MeOH/DCM gradient) to yield Example 956C (250 mg, 29%) as a yellow solid. MS: [M+H]⁺=721.6.

### Example 956D. tert-Butyl (1R,3s,5S)-3-((5-(((R)-1-(4-amino-7-(5-carbamoyl-6-(2,2-difluoroethoxy)pyridin-2-yl)pyrrolo[2,1-f][1,2,4]triazin-5-yl)piperidin-3-yl)carbamoyl)-2-methylthiazol-4-yl)oxy)-8-azabicyclo[3.2.1]octane-8-carboxylate

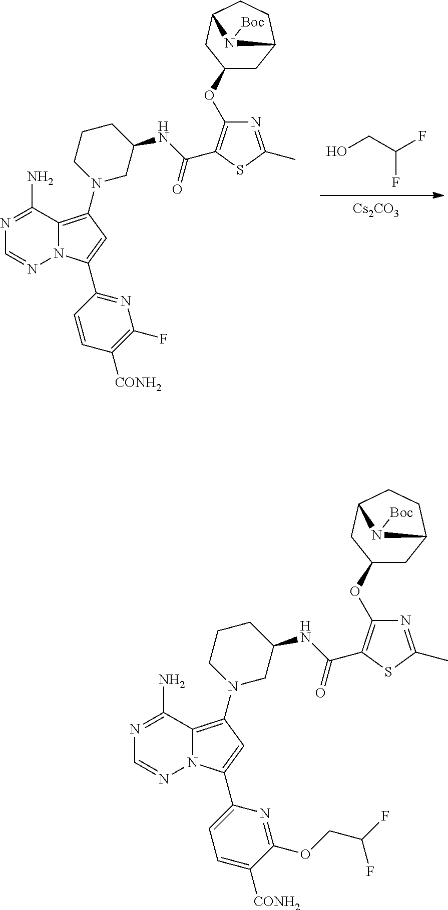 [SMILES: Cc1nc(O[C@@H]2C[C@H]3CC[C@@H](C2)N3C(=O)OC(C)(C)C)c(C(=O)N[C@@H]2CCCN(c3cc(-c4ccc(C(N)=O)c(F)n4)n4ncnc(N)c34)C2)s1 ; "Cs2CO3" ; OCC(F)F ; Cc1nc(O[C@@H]2C[C@H]3CC[C@@H](C2)N3C(=O)OC(C)(C)C)c(C(=O)N[C@@H]2CCCN(c3cc(-c4ccc(C(N)=O)c(OCC(F)F)n4)n4ncnc(N)c34)C2)s1]

To a stirred solution of 3,3-difluoropropan-1-ol (53.3 mg, 0.555 mmol) and Example 956C (100 mg, 0.139 mmol) in DMF (1 mL) was added cesium carbonate (181 mg, 0.555 mmol) and the reaction mixture was heated at 50° C. for 4 h. The reaction mixture was filtered and concentrated in vacuo to yield Example 956D (120 mg, 110%) as a gummy brown solid, which was used without further purification. MS: [M+H]⁺=783.5.

### Example 956E. 4-(((1R,3s,5S)-8-Azabicyclo[3.2.1]octan-3-yl)oxy)-N—((R)-1-(4-amino-7-(5-carbamoyl-6-(2,2-difluoroethoxy)pyridin-2-yl)pyrrolo[2,1-f][1,2,4]triazin-5-yl)piperidin-3-yl)-2-methylthiazole-5-carboxamide

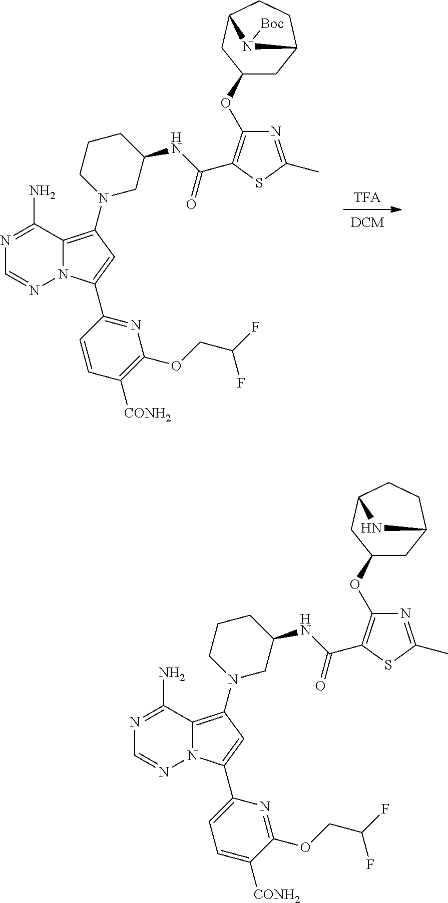

To a stirred solution of Example 956D (120 mg, 0.153 mmol) in DCM was added TFA (0.177 mL, 2.30 mmol). The reaction mixture was stirred at RT for 4 h and concentrated under vacuum to yield Example 956E (100 mg, 96%) as a gummy brown solid. MS: [M+H]⁺=683.6.

### Example 956

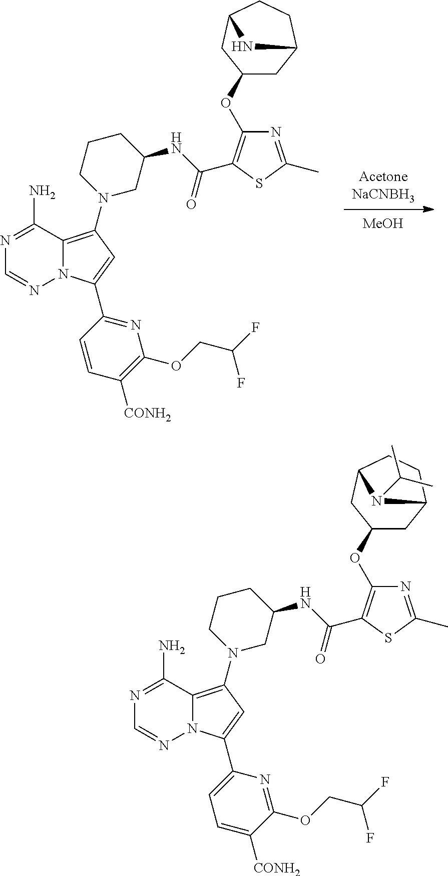 [SMILES: "MeOH" ; Cc1nc(O[C@@H]2C[C@H]3CC[C@@H](C2)N3)c(C(=O)N[C@@H]2CCCN(c3cc(-c4ccc(C(N)=O)c(OCC(F)F)n4)n4ncnc(N)c34)C2)s1 ; "AcetoneNaCNBH3" ; Cc1nc(O[C@@H]2C[C@H]3CC[C@@H](C2)N3C(C)C)c(C(=O)N[C@@H]2CCCN(c3cc(-c4ccc(C(N)=O)c(OCC(F)F)n4)n4ncnc(N)c34)C2)s1]

To a stirred solution of Example 956E (50 mg, 0.073 mmol), acetic acid (0.042 mL, 0.73 mmol) and acetone (0.054 mL, 0.73 mmol) in MeOH (1 mL) at 0° C. was added sodium cyanoborohydride (23.0 mg, 0.366 mmol) and the reaction mixture was stirred at rt for 4 h. Then, it was filtered, concentrated and purified by preparative HPLC to yield Example 956 (21 mg, 39%). LC-MS Method E: RT=1.33 min, [M+H]⁺=725.3; LC-MS Method F: RT=1.06 min, [M+H]⁺=725.3; ¹H NMR (400 MHz, DMSO-d₆) δ ppm 8.45 (d, J=8.0 Hz, 1H), 8.33 (d, J=8.0 Hz, 1H), 7.99 (s, 1H), 7.78 (br s, 1H), 7.52 (s, 1H), 7.39 (s, 1H), 6.79-6.37 (m, 1H), 5.31-5.11 (m, 1H), 4.85 (dt, J=3.1, 14.9 Hz, 2H), 4.31-4.10 (m, 1H), 3.15-2.88 (m, 3H), 2.63 (s, 3H), 1.89-1.79 (m, 4H), 1.04-0.57 (m, 3H).

### Example 957

### Preparation of (R)—N-(1-(7-(1-acetyl-2,5-dihydro-1H-pyrrol-3-yl)-4-aminopyrrolo[2,1-f][1,2,4]triazin-5-yl)piperidin-3-yl)-5-methyl-4-(piperidin-4-yloxy)thiazole-2-carboxamide

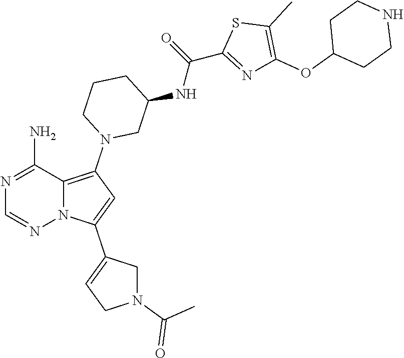 [SMILES: CC(=O)N1CC=C(c2cc(N3CCC[C@@H](NC(=O)c4nc(OC5CCNCC5)c(C)s4)C3)c3c(N)ncnn23)C1]

### Example 957A. Ethyl 4-hydroxy-5-methylthiazole-2-carboxylate

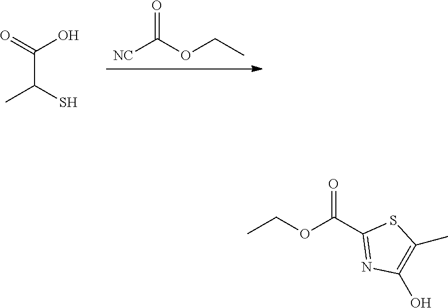

To an ice-cold mixture of 2-mercaptopropanoic acid (4.18 mL, 47.1 mmol) and ethyl carbonocyanidate (4.65 mL, 47.1 mmol) was added pyridine (1.14 mL, 14.1 mmol) dropwise and the mixture was stirred at room temperature for 1 h and then heated at 100° C. for 2 h. After cooling, ethanol (50 mL) was added and stirred at RT for 1 h which led to precipitation of product as solid. The solid was filtered, washed with diethyl ether (2×25 mL) and dried which afforded Example 957A (8.0 g, 83%) as an off-white solid. MS: [M+H]⁺=188.1; ¹H NMR (300 MHz, DMSO-d₆) δ ppm 11.01 (br s, 1H), 4.31 (q, J=6.9 Hz, 2H), 2.26 (s, 3H), 1.30 (t, J=7.2 Hz, 3H).

### Example 957B. Ethyl 4-((1-(tert-butoxycarbonyl) piperidin-4-yl) oxy)-5-methylthiazole-2-carboxylate

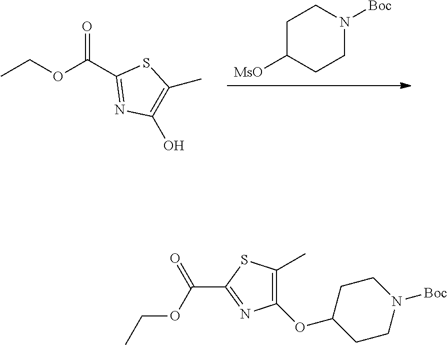 [SMILES: CCOC(=O)c1nc(O)c(C)s1 ; CC(C)(C)OC(=O)N1CCC(OS(C)(=O)=O)CC1 ; CCOC(=O)c1nc(OC2CCN(C(=O)OC(C)(C)C)CC2)c(C)s1]

To a mixture of ethyl 4-hydroxy-5-methylthiazole-2-carboxylate. Example 957A (2.5 g, 13.4 mmol) and K₂CO₃ (4.61 g, 33.4 mmol) in 1,4-dioxane (50 mL) was added tert-butyl 4-((methyl sulfonyl)oxy) piperidine-1-carboxylate (3.73 g, 13.4 mmol) followed by 18-crown-6 (0.353 g, 1.34 mmol) and the mixture was heated at 80° C. for 16 h. After cooling, it was quenched with ice water and diluted with ethyl acetate (50 mL). The organic layer was separated, dried over sodium sulphate and concentrated. The product was purified by flash chromatography (18% ethyl acetate in petroleum ether) to afford Example 957B (4.0 g, 74%) as an off-white solid. MS: [M+H]⁺=371.2; ¹H NMR (300 MHz, CDCl₃) δ ppm 5.03 (td, J=3.9, 7.4 Hz, 1H), 4.42 (q, J=7.2 Hz, 2H), 3.79-3.65 (m, 2H), 3.37-3.23 (m, 2H), 2.33 (s, 3H), 2.01-1.85 (m, 2H), 1.77-1.66 (m, 2H), 1.51-1.44 (m, 9H), 1.44-1.37 (m, 3H).

### Example 957C. 4-((1-(tert-Butoxycarbonyl) piperidin-4-yl) oxy)-5-methylthiazole-2-carboxylic acid

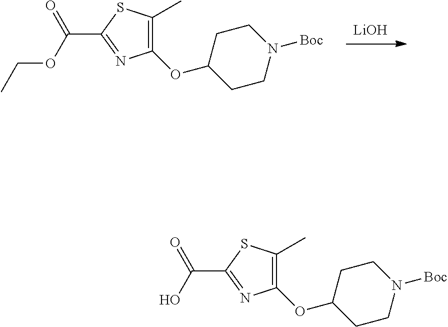 [SMILES: CCOC(=O)c1nc(OC2CCN(C(=O)OC(C)(C)C)CC2)c(C)s1 ; "LiOH" ; Cc1sc(C(=O)O)nc1OC1CCN(C(=O)OC(C)(C)C)CC1]

To a mixture of Example 957B (750 mg, 2.024 mmol) in THF (10 mL) and water (2.5 mL) was added LiOH (145 mg, 6.07 mmol) and it was stirred at rt for 16 h. THF was removed under vacuum and the aqueous layer was washed with diethyl ether (3×5 mL). The aqueous phase was acidified to pH ~2 using 10% aq. citric acid solution and extracted with ethyl acetate (3×10 mL). The combined ethyl acetate layer was washed with brine (10 mL), dried over anhydrous sodium sulphate and concentrated in vacuum to obtain Example 957C (620 mg, 85%) as an off-white solid. MS: [M−H]⁺=341.4.

### Example 957D. tert-Butyl (R)-4-((2-((1-(4-amino-7-bromopyrrolo[2,1-f][1,2,4]triazin-5-yl) piperidin-3-yl) carbamoyl)-5-methylthiazol-4-yl) oxy) piperidine-1-carboxylate

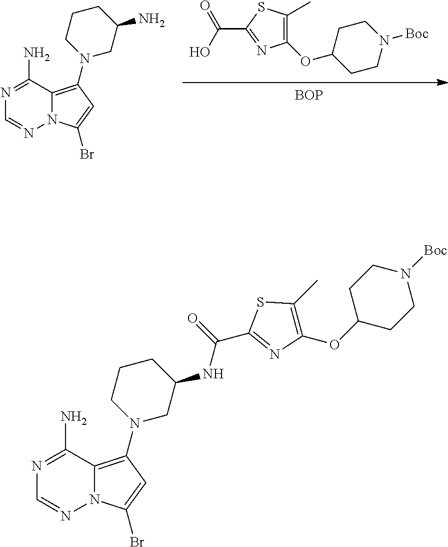

To a mixture of Intermediate 61 (545 mg, 1.75 mmol), Example 957C (600 mg, 1.75 mmol) and BOP (1163 mg, 2.63 mmol) in DMF (10 mL) was added DIEA (0.918 mL, 5.26 mmol) and it was stirred at it for 6 h. The reaction mixture was quenched with ice water and diluted with ethyl acetate (30 mL). The organic layer was separated, washed with water (3×10 mL), dried over anhydrous sodium sulphate, filtered and concentrated to obtain the crude Example 957D (1.1 g, 77%) as a brown semi solid. MS: [M+H]⁺=635.4.

### Example 957E. (R)—N-(1-(4-Amino-7-bromopyrrolo[2,1-f][1,2,4] triazin-5-yl) piperidin-3-yl)-5-methyl-4-(piperidin-4-yloxy) thiazole-2-carboxamide

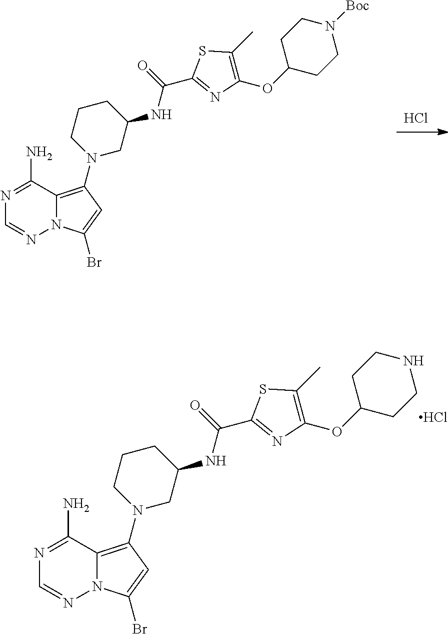 [SMILES: Cc1sc(C(=O)N[C@@H]2CCCN(c3cc(Br)n4ncnc(N)c34)C2)nc1OC1CCN(C(=O)OC(C)(C)C)CC1 ; Cl ; Cc1sc(C(=O)N[C@@H]2CCCN(c3cc(Br)n4ncnc(N)c34)C2)nc1OC1CCNCC1 ; Cl]

To an ice-cold mixture of Example 957D (250 mg, 0.393 mmol) in dichloromethane (4 mL) was added HCl (4M in 1,4-dioxane, 0.036 mL, 1.4 mmol) and the mixture was stirred at room temperature for 6 h. It was concentrated to get the crude product which was triturated with diethyl ether (2×10 mL) and resulting solid was filtered and dried to obtain Example 957E as HCl salt (200 mg, 70%) as an off-white solid. MS: [M+H]⁺=535.3.

### Example 957

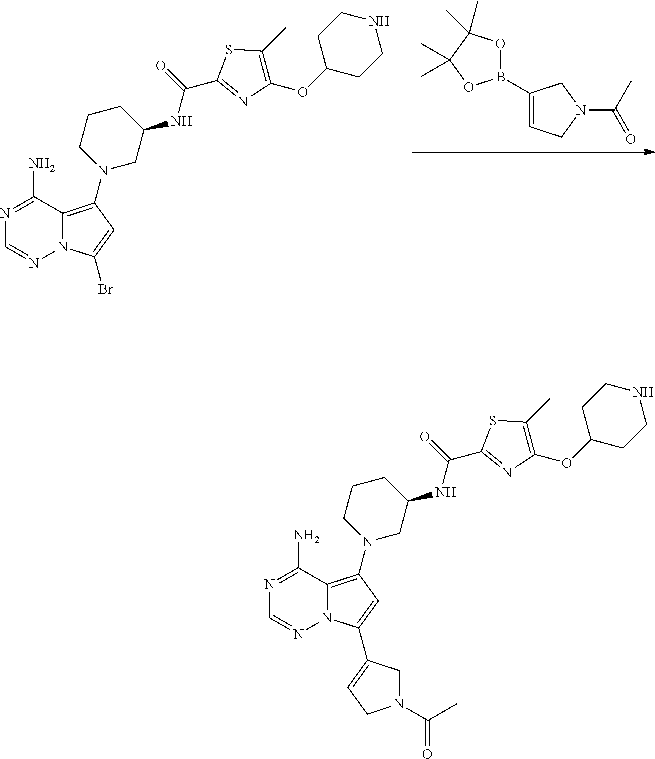

The mixture of Example 957E (100 mg, 0.187 mmol), I-(3-(4,4,5,5-tetramethyl-1,3,2-dioxaborolan-2-yl)-2,5-dihydro-1H-pyrrol-1-yl) ethan-1-one (44.3 mg, 0.187 mmol), potassium phosphate tribasic monohydrate (2M aq. Soln.) (0.233 mL, 0.467 mmol) and XPhos Pd G2 (7.4 mg, 9.3 mmol) in THF (3 mL) was degassed by purging N₂ gas for 5 min. Mixture was heated at 70° C. for 6 h, cooled and diluted with ethyl acetate (10 mL). Water (15 mL) was added, and the organic layer was collected and concentrated in vacuo to get the crude product which was purified by preparative HPLC to obtain Example 957 (18.5 mg, 17%) as an off-white solid. LC-MS Method E: RT=1.13 min, [M+H]⁺=566.3; LC-MS Method F: RT=0.88 min, [M+H]⁺=566.3; ¹H NMR (400 MHz, DMSO-d₆) δ ppm 8.36 (br dd, J=4.9, 8.1 Hz, 1H), 7.87 (d, J=1.8 Hz, 2H), 6.82-6.74 (m, 1H), 6.70 (d, J=14.0 Hz, 1H), 4.79-4.62 (m, 2H), 4.48 (br d, J=9.5 Hz, 2H), 4.26 (br s, 1H), 4.19-4.06 (m, 1H), 3.17-3.14 (m, 1H), 3.04-2.94 (m, 3H), 2.81-2.74 (m, 1H), 2.67-2.58 (m, 3H), 2.28 (s, 3H), 2.03 (d, J=16.0 Hz, 3H), 1.96-1.73 (m, 6H), 1.68-1.48 (m, 3H).

### Example 958

### Preparation of N—((R)-1-(7-(1-acetyl-2,5-dihydro-1H-pyrrol-3-yl)-4-aminopyrrolo[2,1-f][1,2,4]triazin-5-yl) piperidin-3-yl)-5-chloro-3-(((3aR,6aS)-2-methyloctahydrocyclopenta[c]pyrrol-5-yl) oxy) thiophene-2-carboxamide

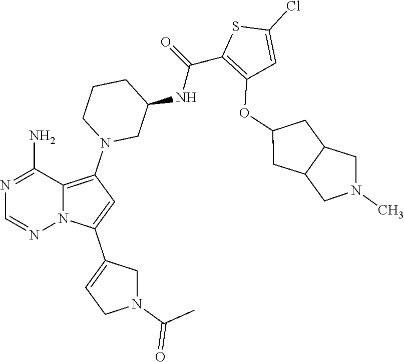 [SMILES: CC(=O)N1CC=C(c2cc(N3CCC[C@@H](NC(=O)c4sc(Cl)cc4OC4CC5CN(C)CC5C4)C3)c3c(N)ncnn23)C1]

### Example 958A. tert-Butyl (3aR,6aS)-5-hydroxyhexahydrocyclopenta[c]pyrrole-2(1H)-carboxylate

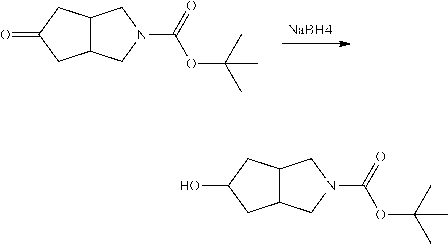 [SMILES: CC(C)(C)OC(=O)N1CC2CC(=O)CC2C1 ; "NaBH4" ; CC(C)(C)OC(=O)N1CC2CC(O)CC2C1]

Analogous to the preparation of Example 946A, reaction of tert-butyl (3aR,6aS)-5-oxohexahydrocyclopenta[c]pyrrole-2(1H)-carboxylate (3 g, 13.3 mmol) and sodium borohydride (1.01 g, 26.6 mmol) afforded Example 958A (2.95 g, 97%) as an off-white solid. MS: [M+H]⁺=228.2; ¹H NMR (300 MHz, CDCl₃) δ ppm 4.36-4.26 (m, 1H), 3.58-3.43 (m, 2H), 3.40-3.28 (m, 2H), 2.69-2.54 (m, 2H), 2.26-2.10 (m, 2H), 1.76-1.59 (m, 2H), 1.56-1.39 (m, 10H).

### Example 958B. tert-Butyl (3aR,6aS)-5-((5-chloro-2-(methoxycarbonyl) thiophen-3-yl) oxy) hexahydrocyclopenta[c]pyrrole-2(1H)-carboxylate

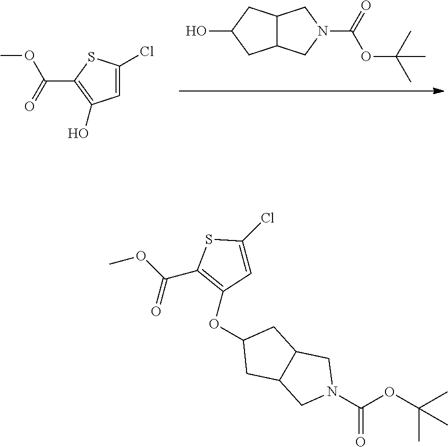 [SMILES: COC(=O)c1sc(Cl)cc1O ; CC(C)(C)OC(=O)N1CC2CC(O)CC2C1 ; COC(=O)c1sc(Cl)cc1OC1CC2CN(C(=O)OC(C)(C)C)CC2C1]

Analogous to the preparation of Example 946B, reaction of methyl 5-chloro-3-hydroxythiophene-2-carboxylate (1.5 g, 5.45 mmol), Example 958A (1.24 g, 5.45 mmol), triphenylphosphine (2.15 g, 8.18 mmol) and DIAD (1.17 mL, 6 mmol) afforded Example 958B (2 g, 88%) as an off-white solid. [M−Boc+2H]⁺=302.0.

### Example 958C. Methyl 5-chloro-3-(((3aR,6aS)-octahydrocyclopenta[c]pyrrol-5-yl) oxy) thiophene-2-carboxylate hydrochloride

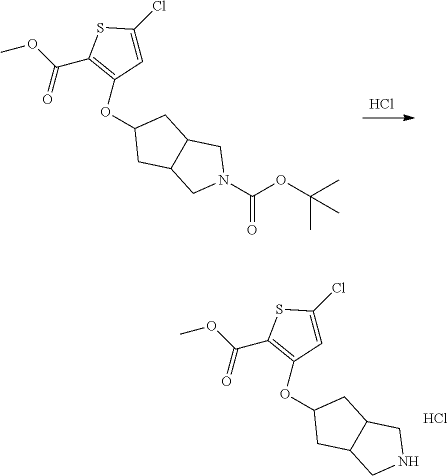

Analogous to the preparation of Example 946C. reaction of Example 958B (2.0 g, 5.0 mmol) and 4M HCl in 1,4-dioxane (3.73 mL, 14.9 mmol) afforded Example 958C as an HCl salt (1.4 g, 82%) as an off-white solid. MS: [M+H]⁺=302.2.

### Example 958D. Methyl 5-chloro-3-(((3aR,6aS)-2-methyloctahydrocyclopenta[c]pyrrol-5-yl) oxy) thiophene-2-carboxylate

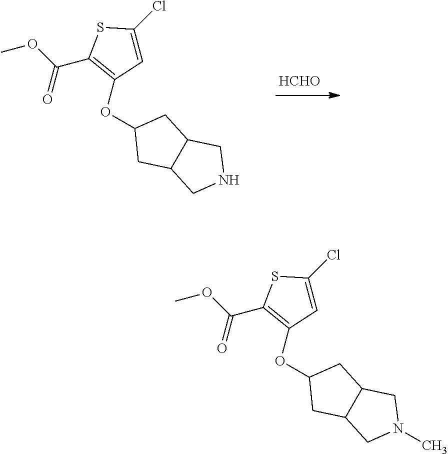

Analogous to the preparation of Example 946D, reaction of Example 958C (600 mg, 1.99 mmol), formaldehyde (36% in water) (0.152 mL, 1.99 mmol), acetic acid (0.011 mL, 0.199 mmol) and sodium cyanoborohydride (187 mg, 2.98 mmol) afforded Example 958D (600 mg, 71%) as a colorless gummy oil. MS: [M+H]⁺=316.0.

### Example 958E. 5-Chloro-3-(((3aR,6aS)-2-methyloctahydrocyclopenta[c]pyrrol-5-yl) oxy) thiophene-2-carboxylic acid

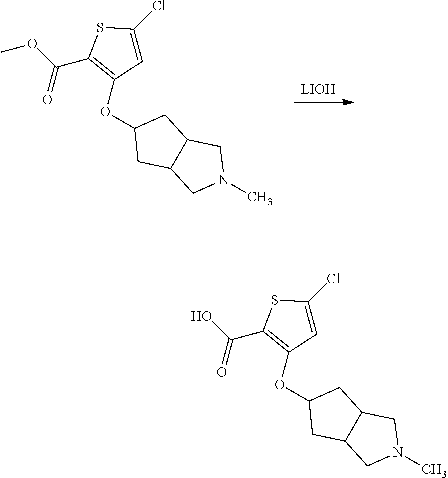

Analogous to the preparation of Example 946E, reaction of Example 958D (400 mg, 1.267 mmol) and LiOH (91 mg, 3.80 mmol) afforded Example 958E (310 mg, 64%) as an off-white solid. MS: [M+H]⁺=302.0.

### Example 958F. N—((R)-1-(4-Amino-7-bromopyrrolo[2,1-f][1,2,4] triazin-5-yl) piperidin-3-yl)-5-chloro-3-(((3aR,6aS)-2-methyloctahydrocyclopenta[c]pyrrol-5-yl) oxy) thiophene-2-carboxamide

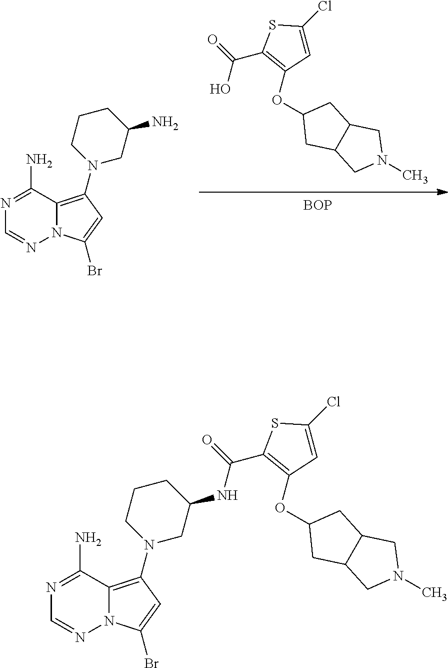 [SMILES: Nc1ncnn2c(Br)cc(N3CCC[C@@H](N)C3)c12 ; CN1CC2CC(Oc3cc(Cl)sc3C(=O)O)CC2C1 ; "BOP" ; CN1CC2CC(Oc3cc(Cl)sc3C(=O)N[C@@H]3CCCN(c4cc(Br)n5ncnc(N)c45)C3)CC2C1]

Analogous to the preparation of Example 946E, reaction of Intermediate 61 (928 mg, 2.98 mmol), Example 958E (900 mg, 2.98 mmol), BOP (1.98 g, 4.47 mmol) and DIEA (1.56 mL, 8.95 mmol) afforded Example 958F (1.5 g, 85%) as a brown semi solid. MS: [M+H]⁺=594.0.

### Example 958

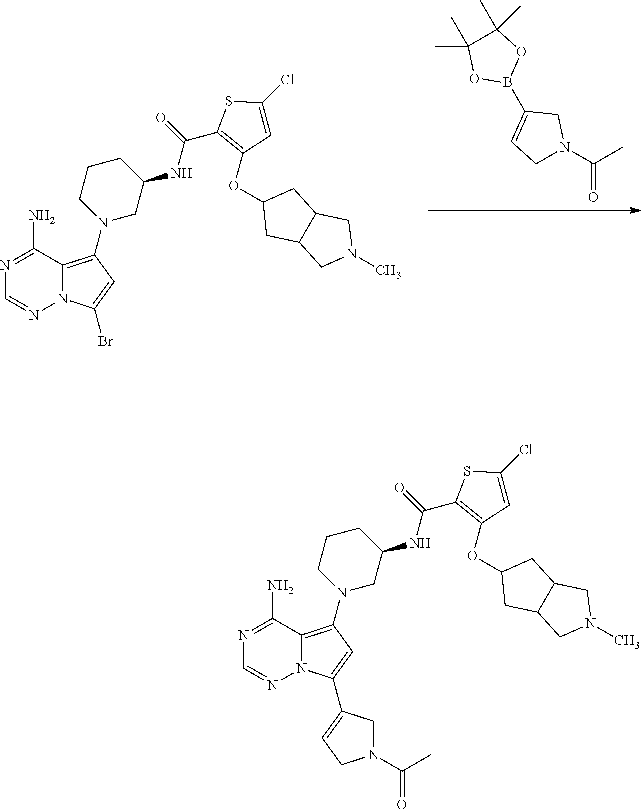 [SMILES: CN1CC2CC(Oc3cc(Cl)sc3C(=O)N[C@@H]3CCCN(c4cc(Br)n5ncnc(N)c45)C3)CC2C1 ; CC(=O)N1CC=C(B2OC(C)(C)C(C)(C)O2)C1 ; CC(=O)N1CC=C(c2cc(N3CCC[C@@H](NC(=O)c4sc(Cl)cc4OC4CC5CN(C)CC5C4)C3)c3c(N)ncnn23)C1]

To a stirred solution of Example 958F (100 mg, 0.094 mmol), 1-(3-(4,4,5,5-tetramethyl-1,3,2-dioxaborolan-2-yl)-2,5-dihydro-1H-pyrrol-1-yl)ethan-1-one (22.3 mg, 0.094 mmol) and potassium phosphate tribasic monohydrate (2M aq. Soln.) (0.118 mL, 0.235 mmol) in THF (5 mL) was added [1,1′-Bis(di-tert-butylphosphino) ferrocene] dichloropalladium(II) (3.1 mg, 4.7 mmol) and the mixture was heated at 70° C. for 6 h. After cooling, the mixture was diluted with ethyl acetate (10 mL) and water (15 mL) was added. Organic layer was collected and concentrated in vacuo to get the crude product which was purified by preparative HPLC to afford Example 958 (16.2 mg, 24.8%) as an off-white solid. LC-MS Method E: RT=1.35 min, [M+H]⁺=625.3, LC-MS Method F: RT=1.01 min, [M+H]⁺=626.3.

### Example 959

### N—((R)-1-(7-(6-(1H-tetrazol-1-yl) pyridin-3-yl)-4-aminopyrrolo[2,1-f][1,2,4] triazin-5-yl) piperidin-3-yl)-5-chloro-3-(((3aR,6aS)-2-isopropyloctahydrocyclopenta[c]pyrrol-5-yl) oxy) thiophene-2-carboxamide

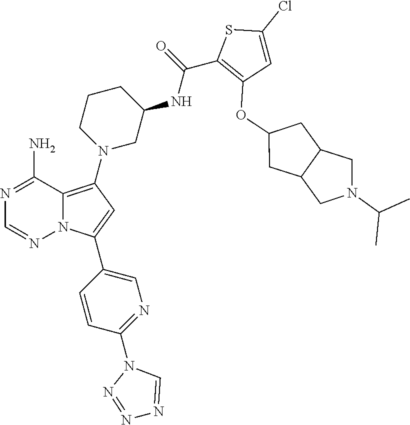 [SMILES: CC(C)N1CC2CC(Oc3cc(Cl)sc3C(=O)N[C@@H]3CCCN(c4cc(-c5ccc(-n6cnnn6)nc5)n5ncnc(N)c45)C3)CC2C1]

Analogous to the preparation of Example 958, Example 959 was prepared. LC-MS Method E: RT=1.64 min, [M+H]⁺=689.2; LC-MS Method F: RT=1.16 min, [M+H]⁺=689.2; ¹H NMR (400 MHz, DMSO-d₆) δ ppm 10.20 (s, 1H), 9.31 (s, 2H), 8.90 (dd, J=1.7, 8.6 Hz, 1H), 8.17 (d, J=8.9 Hz, 2H), 7.96 (s, 1H), 7.41 (d, J=3.6 Hz, 1H), 7.37-6.75 (m, 2H), 3.74-3.58 (m, 1H), 3.27-3.16 (m, 4H), 3.08-2.79 (m, 6H), 2.27-2.19 (m, 1H), 2.02 (br s, 2H), 1.85 (br d, J=4.9 Hz, 6H), 1.64 (br s, 1H), 1.22 (br t, J=7.2 Hz, 6H).

### Example 960

### Preparation of N—((R)-1-(4-amino-7-(3-fluoro-4-(1H-tetrazol-1-yl) phenyl) pyrrolo[2,1-f][1,2,4] triazin-5-yl) piperidin-3-yl)-4-(((1R,3s,5S)-8-(2-hydroxyethyl)-8-azabicyclo [3.2.1] octan-3-yl) oxy)-2-methylthiazole-5-carboxamide

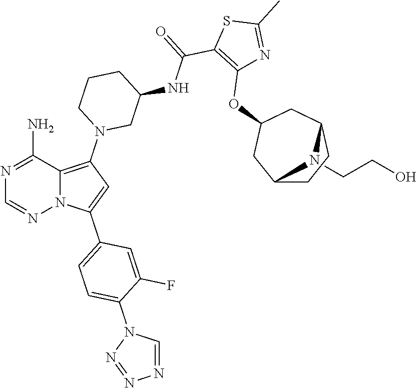 [SMILES: Cc1nc(O[C@@H]2C[C@H]3CC[C@@H](C2)N3CCO)c(C(=O)N[C@@H]2CCCN(c3cc(-c4ccc(-n5cnnn5)c(F)c4)n4ncnc(N)c34)C2)s1]

### Example 960A. Ethyl 4-(((1R,3s,5S)-8-(tert-butoxycarbonyl)-8-azabicyclo [3.2.1] octan-3-yl) oxy)-2-methylthiazole-5-carboxylate

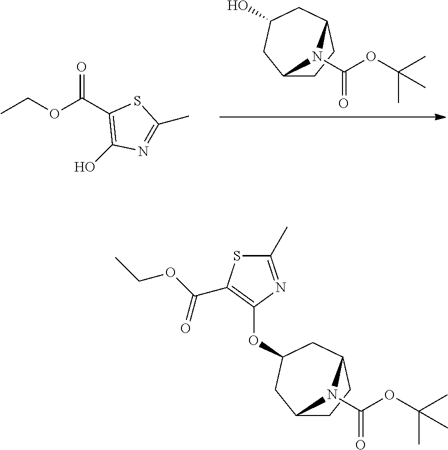 [SMILES: CCOC(=O)c1sc(C)nc1O ; CC(C)(C)OC(=O)N1[C@@H]2CC[C@H]1C[C@@H](O)C2 ; CCOC(=O)c1sc(C)nc1O[C@@H]1C[C@H]2CC[C@@H](C1)N2C(=O)OC(C)(C)C]

To a mixture of 5-thiazolecarboxylic acid 4-hydroxy-2-methyl-ethyl ester (5 g, 26.7 mmol), tert-Butyl 3-endo-3-hydroxy-8-azabicyclo [3.2.1]octane-8-carboxylate (6.07 g, 26.7 mmol) and triphenylphosphine (9.11 g, 34.7 mmol) in THF (100 mL) at room temperature was added dropwise DIAD (6.75 mL, 34.7 mmol) and stirred at room temperature for 16 h. Mixture was concentrated and residue obtained was purified using silica-gel column chromatography (17 to 25% ethyl acetate gradient in petroleum ether) to afford Example 960A (10 g, 80%) as an off-white solid. MS: [M−Boc+2H]⁺=297.1; ¹H NMR (300 MHz, CHCl₃-d) δ ppm 5.26-5.40 (m, 1H) 4.20-4.40 (m, 4H) 2.58-2.63 (m, 3H) 2.12-2.22 (m, 2H) 1.95-2.06 (m, 2H) 1.70-1.80 (m, 2H) 1.51-1.63 (m, 2H) 1.45-1.51 (m, 9H) 1.27-1.35 (m, 3H).

### Example 960B. 4-(((1R,3s,5S)-8-(tert-Butoxycarbonyl)-8-azabicyclo [3.2.1]octan-3-yl) oxy)-2-methylthiazole-5-carboxylic acid

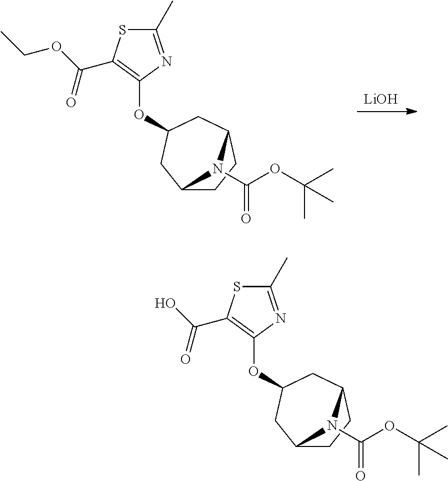

To an ice-cold mixture of Example 960A (14 g, 35.3 mmol) in THF (250 mL) and water (125 mL) was added LiOH (5.07 g, 212 mmol) and the mixture was stirred at room temperature for 16 h. THF was removed under vacuum, aqueous residue obtained was washed with diethyl ether (3×5 mL) and acidified to pH~2 using aq. 10% citric acid solution. The solid precipitated out was filtered and dried to afford Example 960B (12 g, 91%) as an off-white solid. MS: [M+H]⁺=369.35; ¹H NMR (400 MHz, DMSO-d6) δ ppm 12.60-12.30 (m, 1H), 5.35-5.22 (m, 1H), 4.19-4.10 (m, 2H), 2.64-2.57 (m, 3H), 2.18-2.05 (m, 2H), 1.97-1.84 (m, 2H), 1.73-1.66 (m, 2H), 1.65-1.51 (m, 2H), 1.47-1.38 (m, 9H).

### Example 960C. tert-Butyl (1R,3s,5S)-3-((5-(((R)-1-(4-amino-7-bromopyrrolo[2,1-f][1,2,4]triazin-5-yl) piperidin-3-yl) carbamoyl)-2-methylthiazol-4-yl) oxy)-8-azabicyclo [3.2.1] octane-8-carboxylate

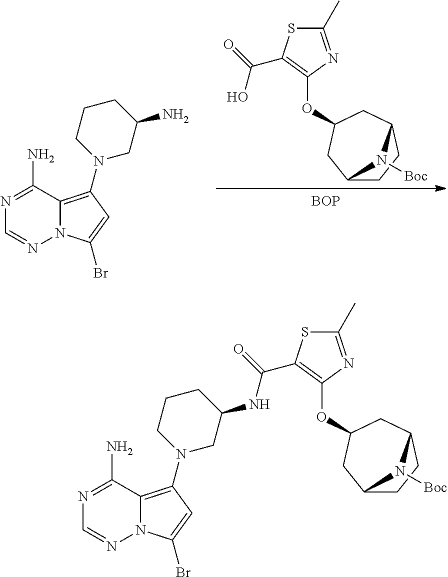 [SMILES: Nc1ncnn2c(Br)cc(N3CCC[C@@H](N)C3)c12 ; Cc1nc(O[C@@H]2C[C@H]3CC[C@@H](C2)N3C(=O)OC(C)(C)C)c(C(=O)O)s1 ; "BOP" ; Cc1nc(O[C@@H]2C[C@H]3CC[C@@H](C2)N3C(=O)OC(C)(C)C)c(C(=O)N[C@@H]2CCCN(c3cc(Br)n4ncnc(N)c34)C2)s1]

To the mixture of Example 960B (2 g, 6.43 mmol), Intermediate 61, HCl salt (2.37 g, 6.43 mmol) and BOP (3.70 g, 8.36 mmol) in DMF (20 mL) was added DIEA (3.37 mL, 19.3 mmol) and it was stirred at room temperature for 1 h. The reaction mixture was diluted with ethyl acetate (60 mL) and water (50 mL) was added. The organic layer was separated, dried over anhydrous sodium sulfate and concentrated in vacuo to get crude product which was purified using silica-gel chromatography (3% MeOH in CHCl₃) to obtain Example 960C (3.6 g, 80%) as an off-white solid. MS: [M+H]⁺=661.25.

### Example 960D. 4-(((1R,3s,5S)-8-Azabicyclo [3.2.1]octan-3-yl) oxy)-N—((R)-1-(4-amino-7-bromopyrrolo[2,1-f][1,2,4]triazin-5-yl) piperidin-3-yl)-2-methylthiazole-5-carboxamide

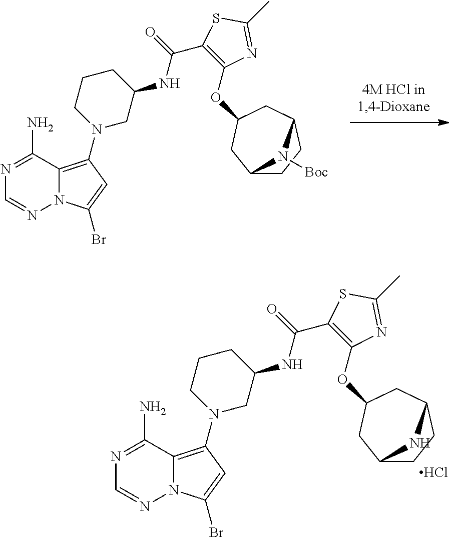 [SMILES: Cc1nc(O[C@@H]2C[C@H]3CC[C@@H](C2)N3C(=O)OC(C)(C)C)c(C(=O)N[C@@H]2CCCN(c3cc(Br)n4ncnc(N)c34)C2)s1 ; "4M HCl in1,4-Dioxane" ; Cc1nc(O[C@@H]2C[C@H]3CC[C@@H](C2)N3)c(C(=O)N[C@@H]2CCCN(c3cc(Br)n4ncnc(N)c34)C2)s1 ; Cl]

To an ice-cold mixture of Example 960C (1.2 g, 0.345 mmol) in dichloromethane (5 mL) was added 4M HCl in 1,4-dioxane (0.26 mL, 1.04 mmol) and the mixture was stirred at room temperature for 6 h. The mixture was concentrated under vacuo to obtain crude product which was triturated with diethyl ether (2×10 mL). The resulting solid was filtered and dried to obtained Example 960D (400 mg, 98%) as pale yellow solid. MS: [M+H]⁺=561.2.

### Example 960E. N—((R)-1-(4-Amino-7-bromopyrrolo[2,1-f][1,2,4] triazin-5-yl) piperidin-3-yl)-4-(((1R,3s,5S)-8-(2-hydroxyethyl)-8-azabicyclo [3.2.1] octan-3-yl) oxy)-2-methylthiazole-5-carboxamide

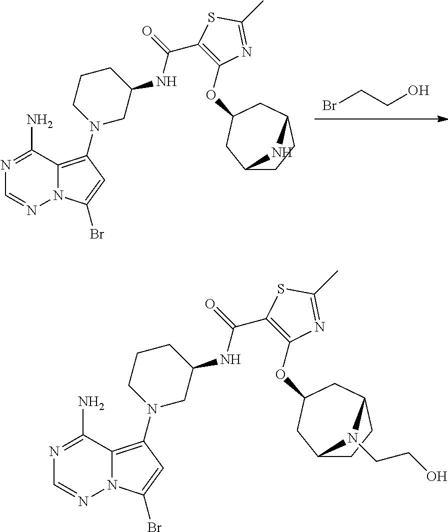

To a mixture of Example 960D (600 mg, 0.506 mmol) and TEA (0.212 mL, 1.52 mmol) in acetonitrile (10 mL) was added 2-bromoethan-1-ol (0.047 mL, 0.66 mmol) and it was stirred at 80° C. for 16 h. After cooling, the reaction mixture was diluted with ethyl acetate (40 mL) and water (30 mL). Organic layer was collected, dried over anhydrous sodium sulphate and concentrated in vacuo to get product Example 960E (300 mg, 65%) as a brown semi solid. MS: [M+H]⁺=605.3.

### Example 960

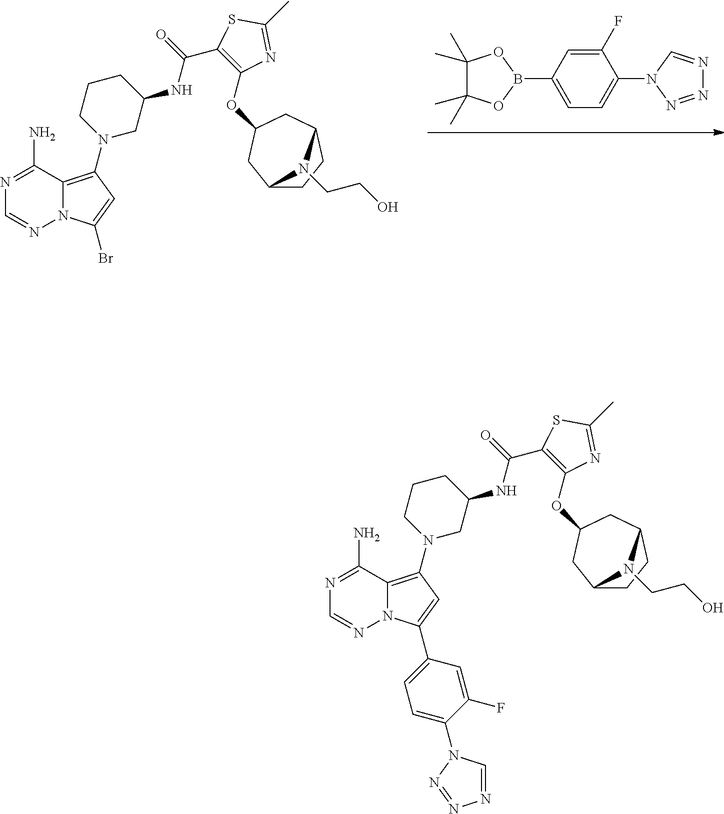 [SMILES: Cc1nc(O[C@@H]2C[C@H]3CC[C@@H](C2)N3CCO)c(C(=O)N[C@@H]2CCCN(c3cc(Br)n4ncnc(N)c34)C2)s1 ; CC1(C)OB(c2ccc(-n3cnnn3)c(F)c2)OC1(C)C ; Cc1nc(O[C@@H]2C[C@H]3CC[C@@H](C2)N3CCO)c(C(=O)N[C@@H]2CCCN(c3cc(-c4ccc(-n5cnnn5)c(F)c4)n4ncnc(N)c34)C2)s1]

To a stirred solution of Example 960E (60 mg, 0.099 mmol), 1-(2-fluoro-4-(4,4,5,5-tetramethyl-1,3,2-dioxaborolan-2-yl) phenyl)-1H-tetrazole (28.7 mg, 0.099 mmol) and potassium phosphate tribasic monohydrate (2M aq., 0.124 mL, 0.248 mmol) in THF (3 mL) was added [1,1′-Bis(di-tert-butylphosphino) ferrocene] dichloropalladium(II) (6.5 mg, 9.9 mmol) and the mixture was degassed with N₂ and heated at 70° C. for 16 h. After cooling, it was diluted with ethyl acetate (10 mL) and water (15 mL) was added. Organic layer was collected and concentrated in vacuo to get crude product which was purified by preparative HPLC to afford Example 960 (3.4 mg, 5%) as an off-white solid. LC-MS Method E: RT=1.60 min, [M+H]⁺=690.2; LC-MS Method F: RT=1.51 min, [M+H]⁺=689.3; ¹H NMR (400 MHz, DMSO-d₆) δ ppm 9.98 (d, J=1.5 Hz, 1H), 9.54 (br s, 1H), 8.43 (dd, J=1.6, 12.8 Hz, 1H), 8.29-8.13 (m, 1H), 8.01-7.87 (m, 1H), 7.36 (s, 1H), 7.23 (s, 1H), 7.10 (s, 1H), 6.98 (s, 1H), 5.50-5.04 (m, 1H), 4.18 (br s, 3H), 3.79-3.73 (m, 2H), 3.28-3.16 (m, 2H), 3.08 (br d, J=4.5 Hz, 3H), 2.90 (br s, 1H), 2.64 (s, 3H), 2.47-2.38 (m, 3H), 2.28-2.10 (m, 4H), 2.04-1.75 (m, 5H), 1.55 (br s, 1H).

The following Examples %61-964 in Table 65 were prepared using the same procedure as shown in Example 960.

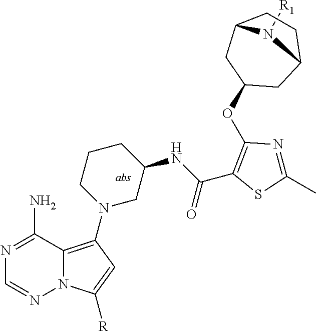

TABLE 65

| | | | | | HPLC | |
|---|---|---|---|---|---|---|
| | | | | LCMS | Method, | |
| Example | R | R1 | Name | (M+H)+ | RT (min.) | 1H NMR |
| 961 | | Me | N-((R)-1-(4-amino-7-(1-oxoisoindolin-5- | 629.3 | E: 1.174 | (400 MHZ, DMSO-d6) 8 8.56 ppm (s, 1H), 8.36 (s, |
| | | | y1)pyrrolo[2,1-f][1,2,4]triazin-5- | | F: 0.759 | 1H), 8.20-8.11 (m, 1H), 7.90 (s, 1H), 7.72 (d, J = 8.0 |
| | | | y1)piperidin-3-y1)-2-methy1-4- | | | Hz, 1H), 7.19 (s, 1H), 6.90 (s, 1H), 5.29-5.14 (m, IH), |
| | | | (((IR,3s,5S)-8-methyl-8- | | | 4.43 (s, 2H), 4.22-4.16 (m, 1H), 3.20-3.14 (m, 1H), |
| | | | azabicyclo[3.2.1 ]octan-3-yl)oxy)thiazole- | | | 3.00 (br s, 4H), 2.63 (s, 3H), 2.19 (s, 3H), 2.11 (s, 2H), |
| | | | 5-carboxamide | | | 1.91 (s, 8H), 1.58 (br s, 3H) |
| 962 | F | iPr | N-((R)-1-(4-amino-7-(4-carbamoyl-3- | 662.3 | E: 1.276 | (400 MHZ, DMSO-d6) 8 ppm 8.13 (dd, J = 1.4, 13.1 |
| | NH2 | | fluorophenyl )pyrrolo[2, 1-f][ 1,2,4]triazin- | | F: 0.974 | Hz, 1H), 8.01 (dd, J = 1.6, 8.1 Hz, 1H), 7.92 (s, IH), |
| | | | 5-y1)piperidin-3-y1)-4-(((1R,3s,5S)-8- | | | 7.74 (t, J = 8.1 Hz, 1H), 7.65 (br d, ) = 15.5 Hz, 2H), |
| | | | isopropyl-8-azabicyclo[3.2.1 ]octan-3- | | | 7.25 (s, 1H), 5.21-5.09 (m, 1H), 4.22-4.62 (br s, 2H), |
| | | | y1)oxy)-2-methylthiazole-5-carboxamide | | | 3.15-2.84 (m, 4H), 2.62 (s, 4H), 1.91 (s, 10H), 1.66- |
| | | | | | | 1.47 (m, 3H), 0.80 (br s, 6H) |
| 963 | N | Me | N-((R)-1-(7-(6-(1/-tetrazol-1-y1)pyridin- | 642.3 | E- 1.202 | H NMR (400 MHZ, DMSO-d.) 6 ppm 10.21 (s, 1 H), |
| | N-N | | 3-y1)-4-aminopyrrolo[2,1-1][1,2,4]triazin- | | F: 1.003 | 9.26-9.35 (m, 1 H), 8.84-8.95 (m, 1 H), 8.65-8.21 (m, |
| | | | 5-y1)piperidin-3-y1)-2-methyl-4- | | | 1 H), 8.21-8.12 (m, 1 H), 7.94 (s, 1 H), 7.36 (3, 2 H), |
| | | | (((IR,35,5S)-8-methy1-8- | | | 7.07-6.57 (m, 1 H), 5.22-5.05 (m, 1 H) 4.14-4.28 (m, |
| | | | azabicyclo[ 3.2 1 ]octan-3-yl )oxy )thiazole- | | | 1 H) 3.21-3.10 (m, 3 H) 3.05-2.78 (m, 4 H) 2.62 (s, 3 |
| | | | 5-carboxamide | | | H) 2.19 (s. 3 H), 2.10-1.93 (m, 4 H), 1.78-1.68 (m, 2 |
| | | | | | | H), 1.63-1.54 (m, 2 H), 1.65-1.45 (m. 3 H). |
| 964 | N | Ethyl | N-((R)-1-(4-amino-7-(6-cyanopyridin-3- | 613.3 | E: 1.42 | (400 MHZ, DMSO-d6) 8 ppm 9.46-9.40 (m, 1H), 8.81 |
| | CN | | yl)pyrrolo[2, 1-f][1,2,4]triazin-5- | | F: 1.04 | (dd, J = 2.3, 8.5 Hz, 1H), 8.51-8.26 (m, 1H), 8.12-8.06 |
| | | | y1)piperidin-3-y1)-4-(((1R,3s,5S)-8-ethyl- | | | (m, 1H), 7.96 (s, 1H), 7.43 (s, 1H), 7.38 (br s, 1H). |
| | | | 8-azabicyclo[3.2.]]octan-3-y])oxy)-2- | | | 6.95-6.71 (m, 1H), 5.26-5.06 (m, 1H), 4.29-4.13 (m, |
| | | | methylthiazole-5-carboxamide | | | 1H), 3.22-3.13 (m, 1H), 3.04-2.84 (m, 3H), 2.61 (s, |
| | | | | | | 3H), 2.40 (br dd, J = 2.6, 5.1 Hz, 1H), 2.10-1.99 (m, |
| | | | | | | 2H), 1.93-1.81 (m, 5H), 1.79-1.68 (m, 2H), 1.59 (br d, |
| | | | | | | J = 5.8 Hz, 3H), 1.02-0.89 (m, 3H) |

### Example 965

### Preparation of (R)—N-(1-(4-amino-7-(3-fluoro-4-(1H-tetrazol-1-yl) phenyl) pyrrolo[2,1-f][1,2,4] triazin-5-yl) piperidin-3-yl)-2-methyl-4-((1,2,2,6,6-pentamethylpiperidin-4-yl) oxy) thiazole-5-carboxamide

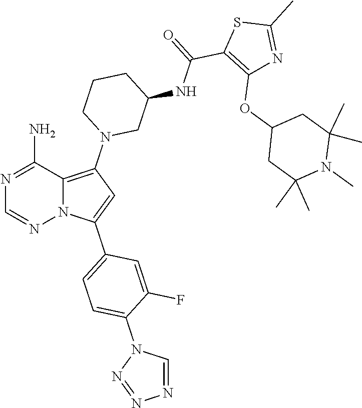 [SMILES: Cc1nc(OC2CC(C)(C)N(C)C(C)(C)C2)c(C(=O)N[C@@H]2CCCN(c3cc(-c4ccc(-n5cnnn5)c(F)c4)n4ncnc(N)c34)C2)s1]

### Example 965A. Ethyl 2-methyl-4-((1,2,2,6,6-pentamethylpiperidin-4-yl) oxy) thiazole-5-carboxylate

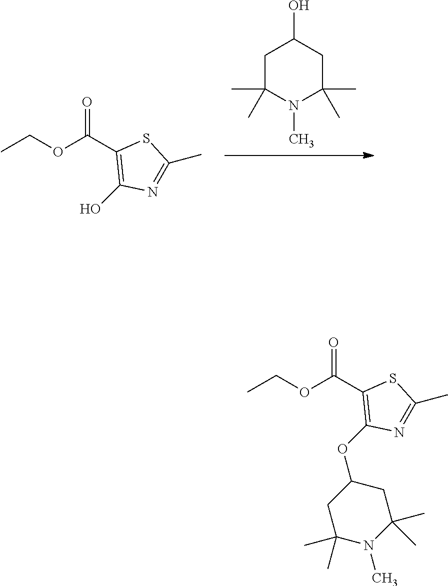

Analogous to the preparation of Example 946B, reaction of 5-thiazolecarboxylic acid 4-hydroxy-2-methyl-ethyl ester (5 g, 26.7 mmol), 1,2,2,6,6-pentamethylpiperidin-4-ol (4.57 g, 26.7 mmol), triphenylphosphine (10.51 g, 40.1 mmol) and DIAD (6.75 mL, 34.7 mmol) afforded Example 965A (5 g, 55%) as an off-white solid. MS: [M+H]⁺=341.25.

### Example 965B. 2-Methyl-4-((1,2,2,6,6-pentamethylpiperidin-4-yl) oxy) thiazole-5-carboxylic acid

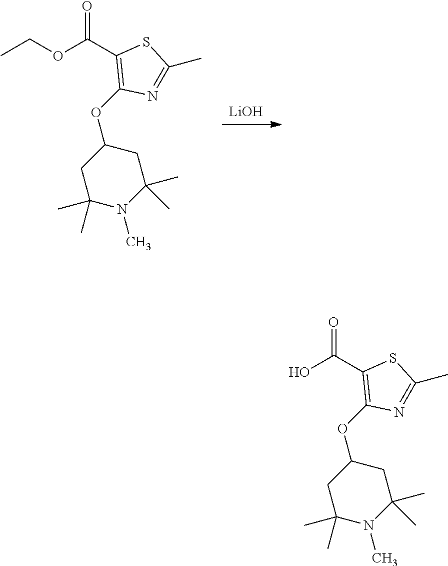

Analogous to the preparation of Example 946E, reaction of Example 965A (500 mg, 1.47 mmol) and LiOH·H₂O (105 mg, 4.41 mmol) afforded Example 965B (600 mg, 98%) as an off-white solid. MS: [M+H]⁺=313.1.

### Example 965C. (R)—N-(1-(4-Amino-7-bromopyrrolo[2,1-f][1,2,4] triazin-5-yl) piperidin-3-yl)-2-methyl-4-((1,2,2,6,6-pentamethylpiperidin-4-yl) oxy) thiazole-5-carboxamide

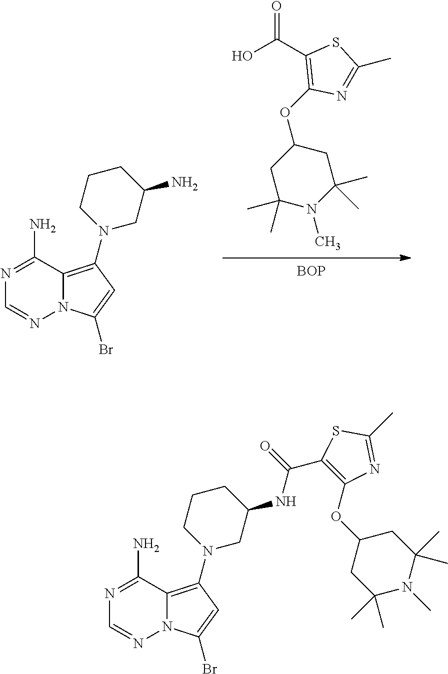

Analogous to the preparation of Example 946, reaction of Intermediate 61 (725 mg, 2.33 mmol), Example 965B (600 mg, 1.44 mmol) and BOP (1340 mg, 3.03 mmol) and DIEA (1.22 mL, 6.99 mmol) afforded Example 965C (1.5 g, 81%) as a brown semi solid. MS: [M−H]⁺=603.4.

### Example 965

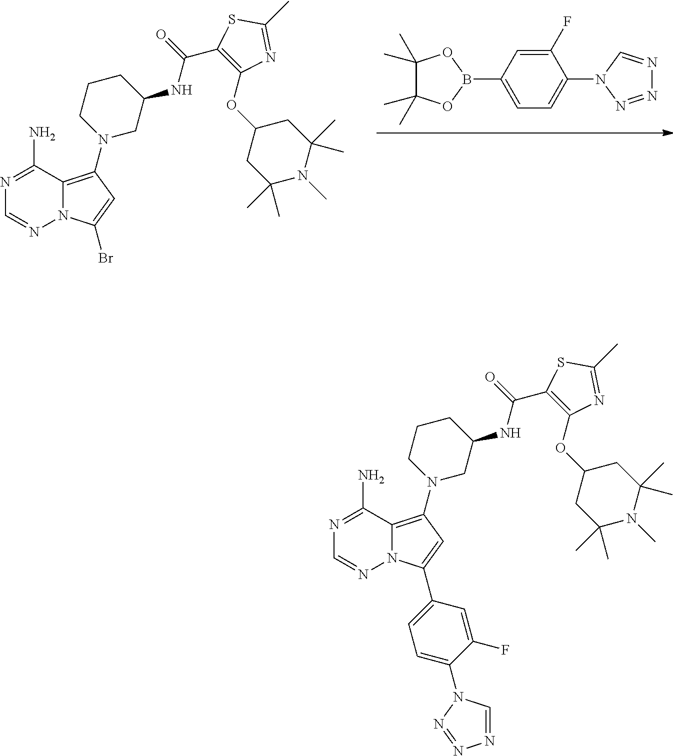 [SMILES: Cc1nc(OC2CC(C)(C)N(C)C(C)(C)C2)c(C(=O)N[C@@H]2CCCN(c3cc(Br)n4ncnc(N)c34)C2)s1 ; CC1(C)OB(c2ccc(-n3cnnn3)c(F)c2)OC1(C)C ; Cc1nc(OC2CC(C)(C)N(C)C(C)(C)C2)c(C(=O)N[C@@H]2CCCN(c3cc(-c4ccc(-n5cnnn5)c(F)c4)n4ncnc(N)c34)C2)s1]

Analogous to the preparation of Example 946, reaction of Example 965C (75 mg, 0.124 mmol), 1-(2-fluoro-4-(4,4,5,5-tetramethyl-1,3,2-dioxaborolan-2-yl) phenyl)-1H-tetrazole (35.9 mg, 0.124 mmol), [1,1′-Bis(di-tert-butylphosphino)ferrocene] dichloropalladium(II) (8.1 mg, 0.012 mmol) and potassium phosphate tribasic (2M aq. soln.) (0.155 mL, 0.310 mmol) afforded Example 965 (4.3 mg, 5%) as an off-white solid. LC-MS Method E: RT=1.79 min, [M+H]⁺=689.3; LC-MS Method F: RT=1.18 min, [M+H]⁺=689.3; ¹H NMR (400 MHz, DMSO-d6) δ ppm 9.97 (s, 1H), 8.44 (br d, J=13.5 Hz, 1H), 8.25 (br d, J=8.0 Hz, 1H), 8.02-7.88 (m, 2H), 7.34 (s, 2H), 5.28-5.05 (m, 1H), 4.16 (br s, 1H), 3.22-3.17 (m, 1H), 2.96 (br s, 2H), 2.62 (s, 3H), 2.31-1.99 (m, 5H), 1.94-1.74 (m, 3H), 1.71-1.39 (m, 2H), 1.18 (br d, J=13.0 Hz, 2H), 1.08 (br s, 12H).

### Example 966

### Preparation of 4-(((1R,3s,5S)-8-azabicyclo [3.2.1]octan-3-yl) oxy)-N—((R)-1-(4-amino-7-(5-oxo-2,3,4,5-tetrahydrobenzo[f][1,4]oxazepin-8-yl) pyrrolo[2,1-f][1,2,4]triazin-5-yl) piperidin-3-yl)-2-methylthiazole-5-carboxamide

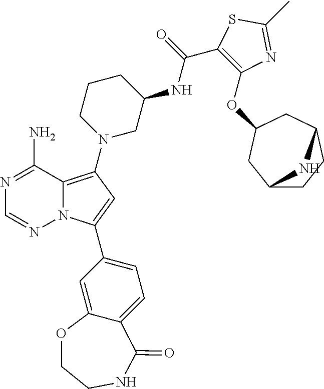 [SMILES: Cc1nc(O[C@@H]2C[C@H]3CC[C@@H](C2)N3)c(C(=O)N[C@@H]2CCCN(c3cc(-c4ccc5c(c4)OCCNC5=O)n4ncnc(N)c34)C2)s1]

### Example 966A. Methyl 4-bromo-2-(2-((tert-butoxycarbonyl) amino) ethoxy) benzoate

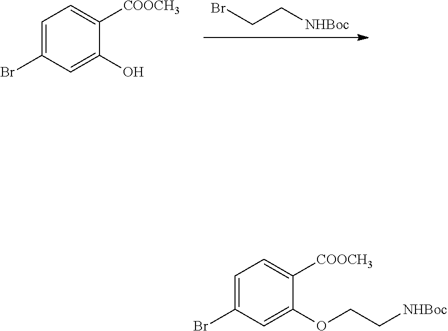 [SMILES: COC(=O)c1ccc(Br)cc1O ; CC(C)(C)OC(=O)NCCBr ; COC(=O)c1ccc(Br)cc1OCCNC(=O)OC(C)(C)C]

To a mixture of methyl 4-bromo-2-hydroxybenzoate (1.0 g, 4.33 mmol) and K₂CO₃ (1.79 g, 13.0 mmol) in 1,4-dioxane (20 mL), was added tert-butyl (2-bromoethyl) carbamate (1.16 g, 5.19 mmol) followed by 18-crown-6 (0.114 g, 0.433 mmol) and the mixture was heated at 70° C. for 16 h. After cooling, it was quenched with water (30 mL) and ethyl acetate (50 mL) was added. Organic layer was separated, dried over anhydrous sodium sulfate and concentrated in vacuo to get the crude product Example 966A (1.5 g, 87%) as an off-white solid. MS: [M−Boc+2H]⁺=274.0.

### Example 966B. 4-Bromo-2-(2-((tert-butoxycarbonyl) amino) ethoxy) benzoic acid

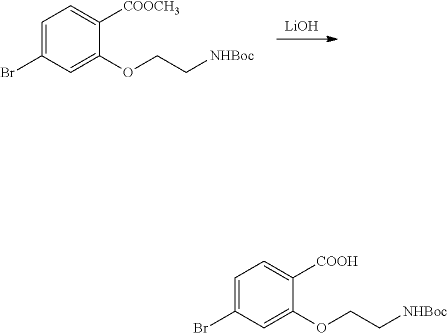 [SMILES: COC(=O)c1ccc(Br)cc1OCCNC(=O)OC(C)(C)C ; "LiOH" ; CC(C)(C)OC(=O)NCCOc1cc(Br)ccc1C(=O)O]

To an ice-cold mixture of Example 966A (1.6 g, 4.28 mmol) in THF (30 mL) and water (10 mL) was added LiOH (0.307 g, 12.8 mmol) and it was stirred at rt for 16 h. THF was removed under vacuum and the aqueous layer was washed with diethyl ether (3×5 mL) followed by acidification to pH~2 using aq. 10% citric acid solution. The solid precipitated out were filtered to afforded Example 966B (1.6 g, 67%) as an off-white solid. MS: [M−H]⁺=360.1.

### Example 966C. 2-(2-Aminoethoxy)-4-bromobenzoic acid hydrochloride

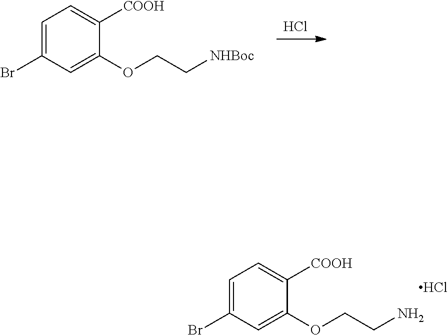 [SMILES: CC(C)(C)OC(=O)NCCOc1cc(Br)ccc1C(=O)O ; Cl ; NCCOc1cc(Br)ccc1C(=O)O ; Cl]

To an ice-cold mixture of Example 966B (500 mg, 1.39 mmol) in 1,4-dioxane (5 mL) was added HCl (4M in 1,4-dioxane) (1.041 mL, 4.16 mmol) and stirred at room temperature for 16 h. The mixture was concentrated under vacuo and triturated with diethyl ether (2×10 mL). The resulting solid was filtered and dried which afforded Example 966C as HCl salt (350 mg, 76%) as an off-white solid. MS: [M+H]⁺+=262.1.

### Example 966D. 8-Bromo-3,4-dihydrobenzo[f][1,4] oxazepin-5(2H)-one

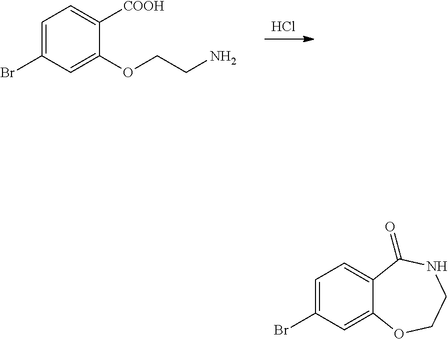 [SMILES: NCCOc1cc(Br)ccc1C(=O)O ; Cl ; O=C1NCCOc2cc(Br)ccc21]

To a mixture of Example 966C (350 mg, 1.346 mmol), BOP (893 mg, 2.02 mmol) in DMF (15 mL) was added DIEA (0.705 mL, 4.04 mmol) and it was stirred at room temperature for 16 h. The reaction mixture was quenched with water (30 mL) and diluted with ethyl acetate (50 mL). The organic layer was collected, dried over anhydrous sodium sulfate and concentrated under vacuo to get Example 966D (300 mg, 76%) as an off-white solid. MS: [M−H]⁺=242.0.

### Example 966E. 8-(4,4,5,5-Tetramethyl-1,3,2-dioxaborolan-2-yl)-3,4-dihydrobenzo[f][1,4] oxazepin-5(2H)-one

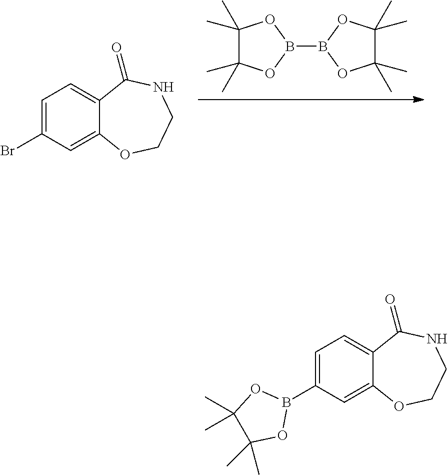

To a mixture of Example 966D (250 mg, 1.03 mmol), bis(pinacolato)diboron (393 mg, 1.55 mmol) and potassium acetate (253 mg, 2.58 mmol) in 1,4-dioxane (20 mL) was added PdCl₂(dppf)-CH₂Cl₂ adduct (84 mg, 0.103 mmol) and it was heated at 90° C. for 6 h. After cooling, the mixture was diluted with ethyl acetate (15 mL) and water (20 mL) was added. Organic layer collected, dried over Na₂SO₄ and concentrated to get Example 966E (500 mg, 38%) as a brown semi solid. MS: [M+H]⁺=290.2.

### Example 966F. tert-Butyl (1R,3s,5S)-3-((5-(((R)-1-(4-amino-7-(5-oxo-2,3,4,5-tetrahydrobenzo[f] [1,4] oxazepin-8-yl) pyrrolo[2,1-f][1,2,4] triazin-5-yl) piperidin-3-yl) carbamoyl)-2-methylthiazol-4-yl) oxy)-8-azabicyclo [3.2.1] octane-8-carboxylate

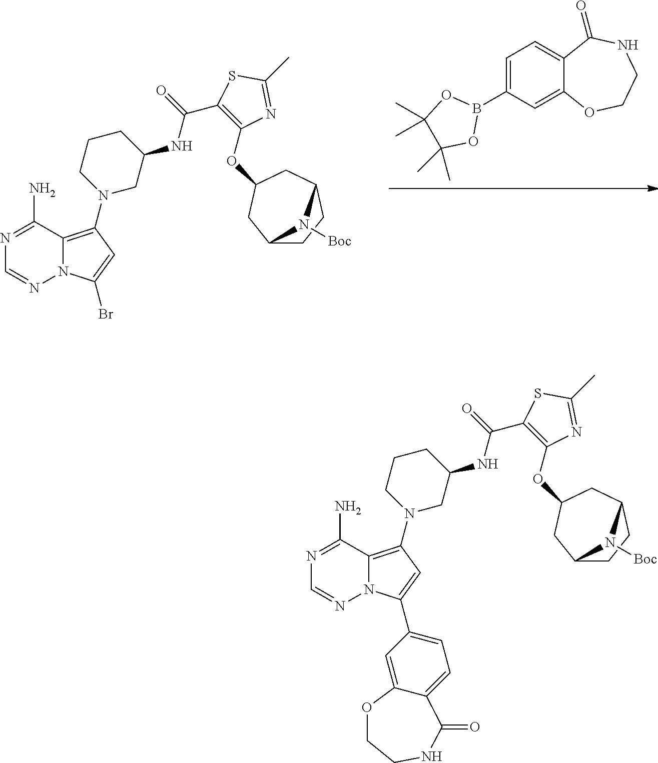 [SMILES: Cc1nc(O[C@@H]2C[C@H]3CC[C@@H](C2)N3C(=O)OC(C)(C)C)c(C(=O)N[C@@H]2CCCN(c3cc(Br)n4ncnc(N)c34)C2)s1 ; CC1(C)OB(c2ccc3c(c2)OCCNC3=O)OC1(C)C ; Cc1nc(O[C@@H]2C[C@H]3CC[C@@H](C2)N3C(=O)OC(C)(C)C)c(C(=O)N[C@@H]2CCCN(c3cc(-c4ccc5c(c4)OCCNC5=O)n4ncnc(N)c34)C2)s1]

Analogous to the preparation of Example 947, reaction of tert-butyl (1R,3s,5S)-3-((5-(((R)-1-(4-amino-7-bromopyrrolo[2,1-f][1,2,4]triazin-5-yl)piperidin-3-yl)carbamoyl)-2-methylthiazol-4-yl)oxy)-8-azabicyclo[3.2.1]octane-8-carboxylate (75 mg, 0.113 mmol), Example 966E (32.8 mg, 0.113 mmol), potassium phosphate tribasic (2M aq. Soln.) (0.142 mL, 0.283 mmol) and [1,1′-Bis(di-tert-butylphosphino) ferrocene]dichloropalladium(II) (7.4 mg, 0.011 mmol) afforded Example 966F (125 mg, 92%) as a brown solid. MS: [M+H]⁺=744.3.

### Example 966

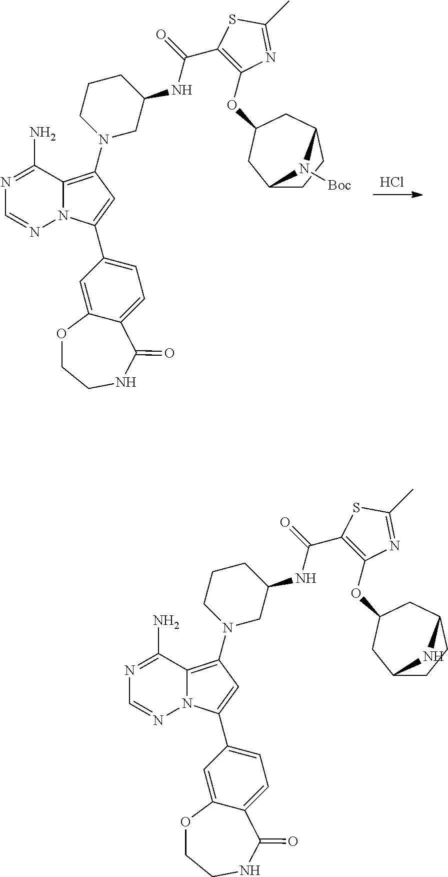 [SMILES: Cc1nc(O[C@@H]2C[C@H]3CC[C@@H](C2)N3C(=O)OC(C)(C)C)c(C(=O)N[C@@H]2CCCN(c3cc(-c4ccc5c(c4)OCCNC5=O)n4ncnc(N)c34)C2)s1 ; Cl ; Cc1nc(O[C@@H]2C[C@H]3CC[C@@H](C2)N3)c(C(=O)N[C@@H]2CCCN(c3cc(-c4ccc5c(c4)OCCNC5=O)n4ncnc(N)c34)C2)s1]

To an ice-cold mixture of Example 966F (100 mg, 0.134 mmol) in dichloromethane (5 mL) at 0° C. was added trifluoroacetic acid (0.031 mL, 0.403 mmol) and stirred at room temperature for 3 h. The reaction mixture was concentrated and purified by preparative HPLC to afford Example 966 (33.9 mg, 37%) as an off-white solid. LC-MS Method E: RT=1.17 min, [M+H]⁺=644.3; LC-MS Method F: RT=0.92 min, [M+H]⁺=644.3; ¹H NMR (400 MHz, DMSO-d₆) δ ppm 8.32 (t, J=5.4 Hz, 1H), 7.92 (d, J=1.5 Hz, 1H), 7.90 (s, 1H), 7.87-7.83 (m, 1H), 7.82-7.75 (m, 1H), 7.19 (s, 1H), 5.20 (br s, 1H), 4.37-4.29 (m, 2H), 4.19 (br s, 1H), 3.85 (br s, 2H), 3.15 (s, 1H), 2.98 (br s, 1H), 2.62 (s, 3H), 2.28 (br s, 2H), 1.91 (s, 10H).

### Example 967

### Preparation of N—((R)-1-(4-amino-7-(2-(tert-butyl)-1,1-dioxido-2,3-dihydrobenzo[d]isothiazol-5-yl)pyrrolo[2,1-f][1,2,4]triazin-5-yl)piperidin-3-yl)-5-chloro-3-(((1R,3s,5S)-8-methyl-8-azabicyclo [3.2.1]octan-3-yl)oxy)thiophene-2-carboxamide

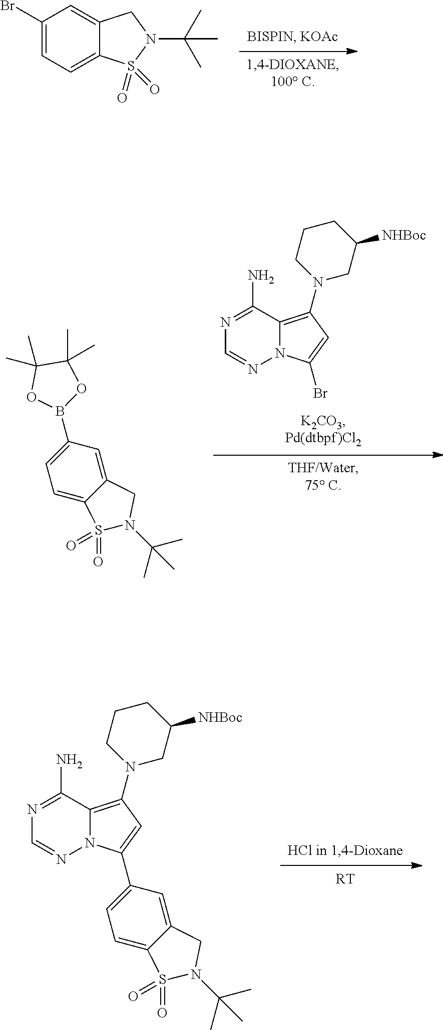

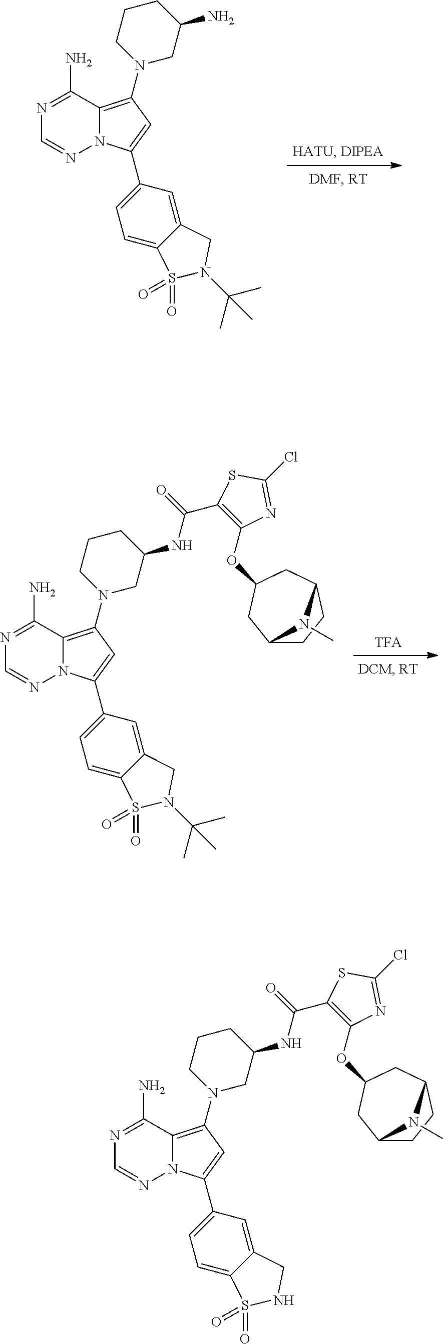

### Example 967A. 2-(tert-butyl)-5-(4,4,5,5-tetramethyl-1,3,2-dioxaborolan-2-yl)-2,3-dihydrobenzo [d]isothiazole 1,1-dioxide

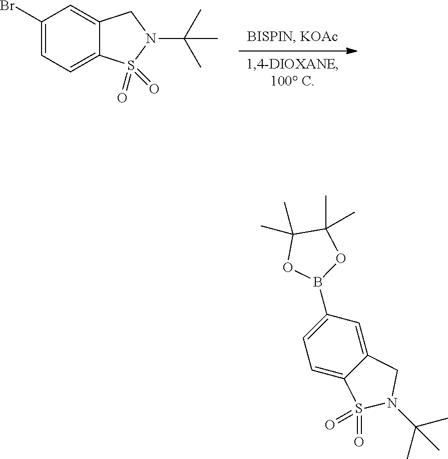

A mixture of 5-bromo-2-(tert-butyl)-2,3-dihydrobenzo[d]isothiazole 1,1-dioxide [PCT Int. Appl., 2016064935, 28 Apr. 2016](0.20 g, 0.657 mmol), bis(pinacolato)diboron (0.200 g, 0.789 mmol) and potassium acetate (0.097 g, 0.986 mmol) in DME (4 mL) was degassed by purging N₂. Then, PdCl₂(dppf)-CH₂Cl₂ adduct (0.027 g, 0.033 mmol) was added and the mixture was heated at 100° C. for 2 h. After cooling, the reaction mixture was diluted with ethyl acetate and filtered through Celite. Ethyl acetate layer was collected, washed with water and concentrated in vacuo to afford 0.3 g of Example 967A as a brown gummy solid. This crude product was taken up as such for the next step without further purification. MS: [M+H]⁺=352.4.

### Example 967B. tert-Butyl (R)-(1-(4-amino-7-(2-(tert-butyl)-1,1-dioxido-2,3-dihydrobenzo[d]isothiazol-5-yl)pyrrolo[2,1-f][1,2,4]triazin-5-yl)piperidin-3-yl) carbamate

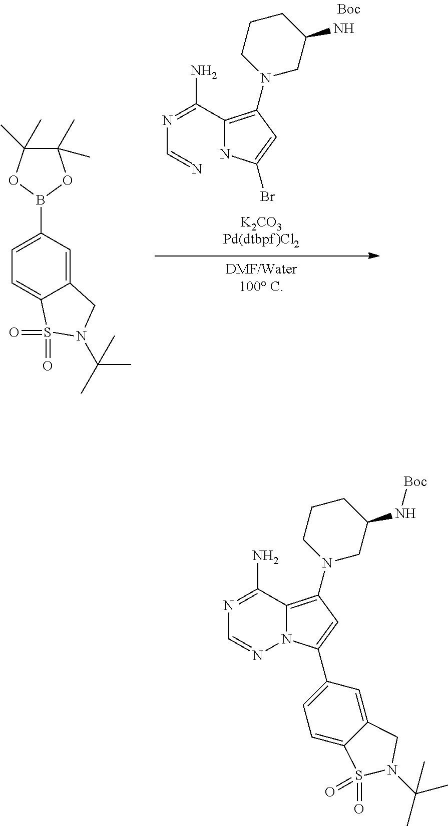

A mixture of Intermediate 29 (0.263 g, 0.641 mmol), K₂CO₃ (0.197 g, 1.42 mmol) and Example 967A (0.25 g, 0.712 mmol) in DMF (4 mL) and water (0.25 mL) was purged with N₂ followed by the addition of PdCl₂(dppf)-CH₂Cl₂ adduct (0.029 g, 0.036 mmol) at rt. Then, the reaction mixture was heated at 100° C. for 18 h. Filtered through Celite after cooling to rt and washed with ethyl acetate 50 mL. Ethyl acetate layer was collected, washed with water and concentrated to get crude product which was purified by flash chromatography (40% to 100% EtOAc/Pet ether gradient) to afford Example 967B (150 mg, 38%) as a brown gummy solid. MS: [M+H]⁺=556.5.

### Example 967C. (R)-5-(4-Amino-5-(3-aminopiperidin-1-yl)pyrrolo[2,1-f][1,2,4]triazin-7-yl)-2-tert-butyl)-2,3-dihydrobenzo[d]isothiazole 1,1-dioxide hydrochloride

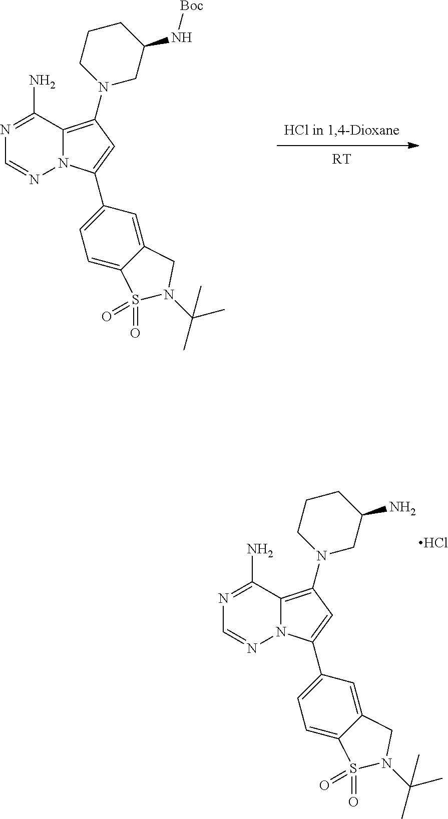 [SMILES: CC(C)(C)OC(=O)N[C@@H]1CCCN(c2cc(-c3ccc4c(c3)CN(C(C)(C)C)S4(=O)=O)n3ncnc(N)c23)C1 ; "RT" ; "HCl in 1,4-Dioxane" ; CC(C)(C)N1Cc2cc(-c3cc(N4CCC[C@@H](N)C4)c4c(N)ncnn34)ccc2S1(=O)=O ; Cl]

To a solution of Example 967B (0.25 g, 0.45 mmol) in 1,4-dioxane (10 mL) was added HCl in 1,4-dioxane (4M, 1.13 mL, 4.50 mmol) at rt and mixture was stirred for 2 h. The reaction mixture was concentrated in vacuo to afford Example 967C as an HCl salt (0.15 g, 73%). MS: [M+H]⁺=456.4.

### Example 967D. N—((R)-1-(4-Amino-7-(2-(tert-butyl)-1,1-dioxido-2,3-dihydrobenzo[d]isothiazol-5-yl)pyrrolo[2,1-f][1,2,4]triazin-5-yl)piperidin-3-yl)-5-methyl-3-(((1R,3s,5S)-8-methyl-8-azabicyclo[3.2.1]octan-3-yl)oxy)thiophene-2-carboxamide

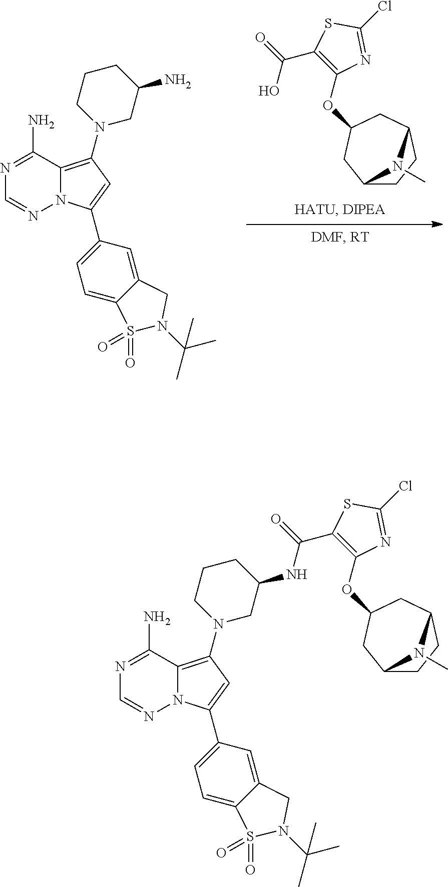

To a stirred solution of 5-chloro-3-(((1R,3s,5S)-8-methyl-8-azabicyclo[3.2.1]octan-3-yl)oxy)thiophene-2-carboxylic acid (0.099 g, 0.329 mmol), HATU (0.150 g, 0.395 mmol) and DIEA (0.144 mL, 0.823 mmol) in DMF (10 mL) was added Example 967C (0.15 g, 0.329 mmol) and it was stirred at rt for 12 h. Water (15 mL) was added and the precipitated solid was filtered which was purified by preparative HPLC to afford Example 967D (0.200 g, 82%) as a brown solid. MS: [M+H]⁺=720.4.

### Example 967

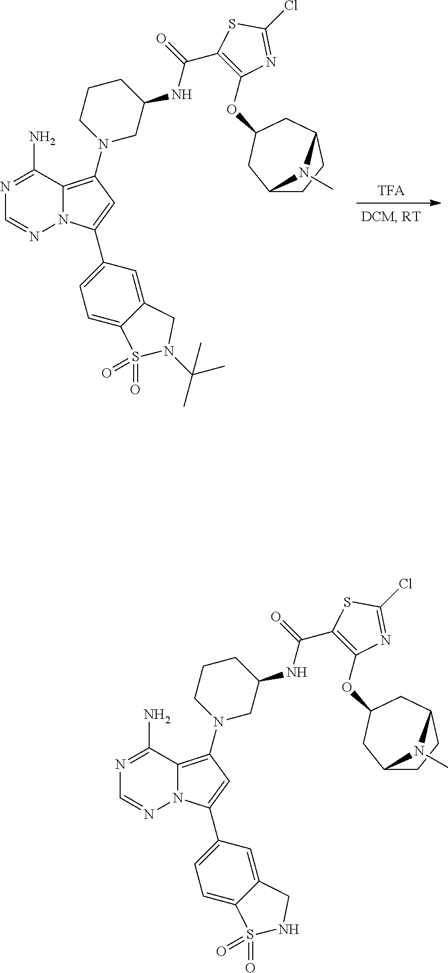

To a solution of Example 967D (0.05 g, 0.068 mmol) in DCM (2 mL) at 0° C. was added TFA (0.052 mL, 0.676 mmol) and mixture was stirred at rt for 12 h. Then, the reaction mixture was concentrated and purified by preparative HPLC to afford Example 967 (4.2 mg, 9%). LCMS: Method F: RT=0.96 min, [M+H]⁺=684.2; ¹H NMR (400 MHz, DMSO-d6) δ ppm 9.85 (br s, 1H), 8.35-8.23 (m, 2H), 8.13 (s, 1H), 7.91 (s, 1H), 7.87 (d, J=8.3 Hz, 1H), 7.84-7.79 (m, 1H), 7.57 (s, 1H), 7.22 (s, 2H), 6.98 (s, 1H), 4.96-4.79 (m, 1H), 4.45 (d, J=5.0 Hz, 2H), 4.17 (br s, 1H), 3.96 (br s, 2H), 3.17 (s, 1H), 3.06 (br s, 1H), 2.86 (s, 1H), 2.69 (d, J=5.0 Hz, 3H), 2.42-2.33 (m, 2H), 2.27-2.09 (m, 3H), 2.01 (br dd, J=1.6, 10.4 Hz, 5H), 1.52 (s, 1H).

### Example 968

### Preparation of N—((R)-1-(4-amino-7-(1-(2,2-difluoroethyl)-5-fluoro-6-oxo-1,6-dihydropyridin-3-yl)pyrrolo[2,1-f][1,2,4]triazin-5-yl)piperidin-3-yl)-5-chloro-3-(((1R,3s,5S)-9-isopropyl-9-azabicyclo[3.3.1]nonan-3-yl)oxy)thiophene-2-carboxamide

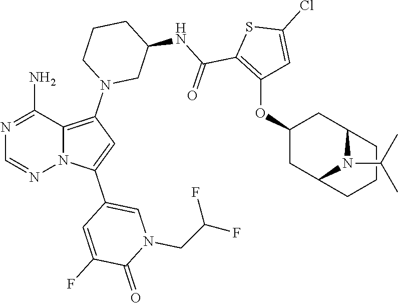 [SMILES: CC(C)N1[C@@H]2CCC[C@H]1C[C@H](Oc1cc(Cl)sc1C(=O)N[C@@H]1CCCN(c3cc(-c4cc(F)c(=O)n(CC(F)F)c4)n4ncnc(N)c34)C1)C2]

### Example 968A. 5-Bromo-1-(2,2-difluoroethyl)-3-fluoropyridin-2(1H)-one

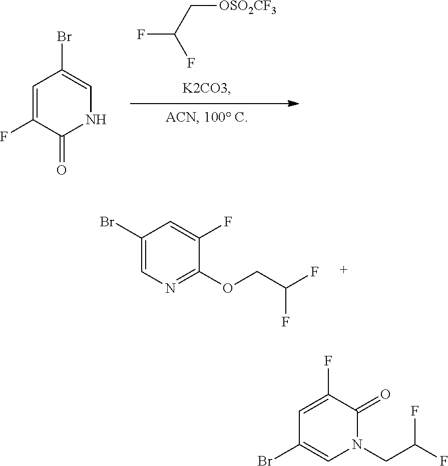

To a mixture of 5-bromo-3-fluoropyridin-2(1H)-one (2.0 g, 10.4 mmol) and K₂CO₃ (2.16 g, 15.6 mmol) in acetonitrile (20 mL) was added 2,2-difluoroethyl trifluoromethanesulfonate (2.23 g, 10.4 mmol) and stirred at 80° C. for 16 h. The reaction mixture was filtered through Celite, and the filtrate was concentrated. The product was purified by silica-gel chromatography to get 5-bromo-2-(2,2-difluoroethoxy)-3-fluoropyridine (400 mg, 15%) and Example 968A (800 mg, 30%) as an off-white solid and colorless liquid, respectively. MS: [M+H]⁺=255.9.

### Example 968B. 1-(2,2-Difluoroethyl)-3-fluoro-5-(4,4,5,5-tetramethyl-1,3,2-dioxaborolan-2-yl)pyridin-2(1H)-one

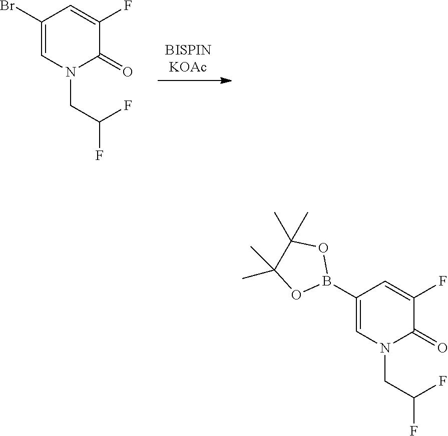

The mixture of Example 968A (500 mg, 2.60 mmol), bis(pinacolato)diboron (794 mg, 3.13 mmol) and potassium acetate (383 mg, 3.91 mmol) in DME (20 mL) was purged with N₂ for 15 min. Then, PdCl₂(dppf)-CH₂Cl₂ adduct (106 mg, 0.130 mmol) was added and heated at 100° C. for 18 h. The reaction mixture was filtered through Celite and concentrated in vacuo to get the crude 0.840 g of Example 968B as a brown gummy solid. MS: [M+H]⁺=304.3.

### Example 698C. tert-Butyl (R)-(1-(4-amino-7-(1-(2,2-difluoroethyl)-5-fluoro-6-oxo-1,6-dihydropyridin-3-yl)pyrrolo[2,1-f][1,2,4]triazin-5-yl)piperidin-3-yl)carbamate

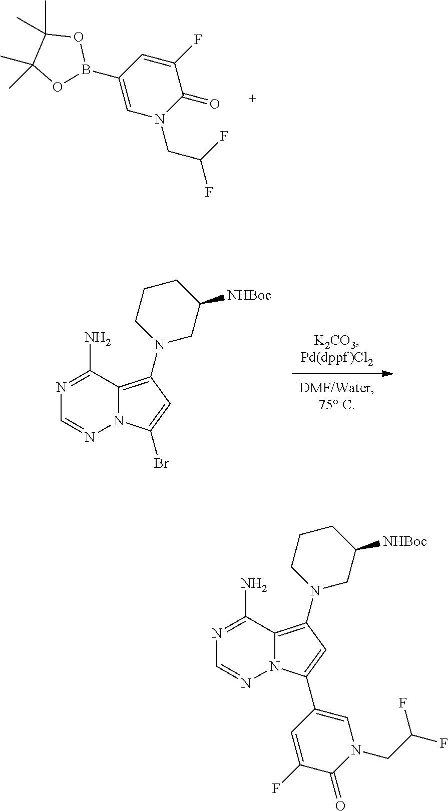 [SMILES: CC1(C)OB(c2cc(F)c(=O)n(CC(F)F)c2)OC1(C)C ; CC(C)(C)OC(=O)N[C@@H]1CCCN(c2cc(Br)n3ncnc(N)c23)C1 ; "DMF/Water,75° C." ; "K2CO3,Pd(dppf)Cl2" ; CC(C)(C)OC(=O)N[C@@H]1CCCN(c2cc(-c3cc(F)c(=O)n(CC(F)F)c3)n3ncnc(N)c23)C1]

Analogous to the preparation of Example 947, reaction of Intermediate 29 (0.45 g, 1.10 mmol), Example 698B (0.5 g, 1.65 mmol), K₂CO₃ (0.304 g, 2.20 mmol) and PdCl₂(dppf)-CH₂Cl₂ adduct (0.045 g, 0.055 mmol) afforded Example 968C (400 mg, 72%) as a brown solid. MS: [M+H]⁺=508.4.

### Example 968D. (R)-5-(4-Amino-5-(3-aminopiperidin-1-yl)pyrrolo[2,1-f][1,2,4]triazin-7-yl)-1-(2,2-difluoroethyl)-3-fluoropyridin-2(1H)-one

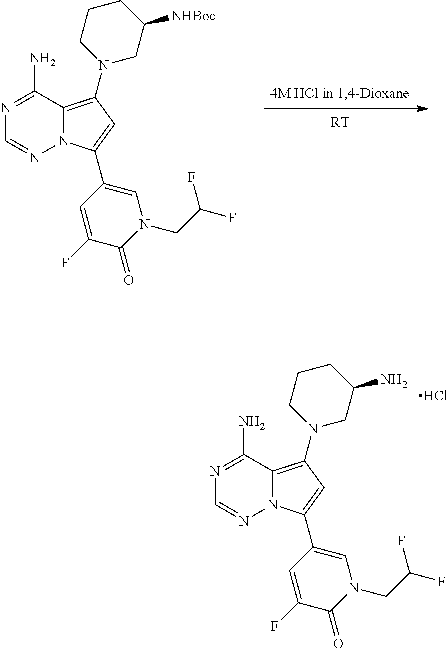 [SMILES: CC(C)(C)OC(=O)N[C@@H]1CCCN(c2cc(-c3cc(F)c(=O)n(CC(F)F)c3)n3ncnc(N)c23)C1 ; "RT" ; "4M HCl in 1,4-Dioxane" ; Nc1ncnn2c(-c3cc(F)c(=O)n(CC(F)F)c3)cc(N3CCC[C@@H](N)C3)c12 ; Cl]

Analogous to the preparation of Example 946C, reaction of Example 968C (1.0 g, 1.97 mmol) and 4M HCl in 1,4-dioxane afforded Example 968D as an off-white solid. MS: [M+H]⁺=408.4.

### Example 968E. tert-Butyl (1R,3s,5S)-3-((5-chloro-2-(methoxycarbonyl)thiophen-3-yl)oxy)-9-azabicyclo[3.3.1]nonane-9-carboxylate

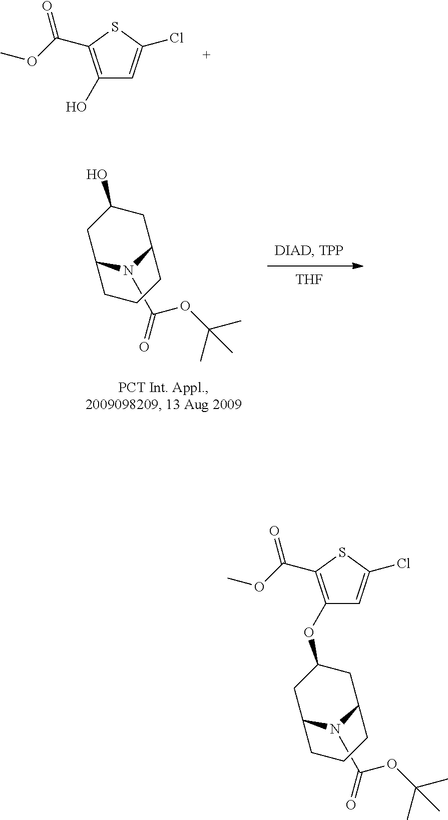

Analogous to the preparation of Example 946, reaction of tert-butyl 3-hydroxy-9-azabicyclo[3.3.1]nonane-9-carboxylate (1.0 g, 4.14 mmol), methyl 5-chloro-3-hydroxythiophene-2-carboxylate (0.798 g, 4.14 mmol), triphenylphosphine (1.30 g, 4.97 mmol) and DIAD (0.967 mL, 4.97 mmol) afforded Example 968E (1.5 g, 87%) as an off-white solid. MS: [M−H]⁺=416.2.

### Example 968F. 3-(((1R,3s,5S)-9-(tert-Butoxycarbonyl)-9-azabicyclo[3.3.1]nonan-3-yl)oxy-5-chlorothiophene-2-carboxylic acid

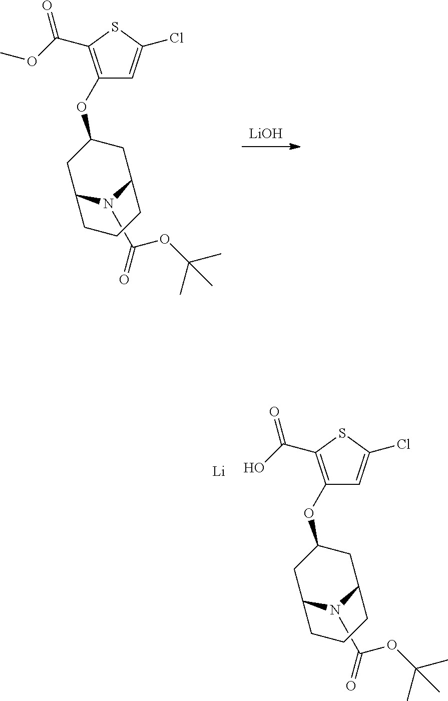

Analogous to the preparation of Example 946E, reaction of Example 968E (1.0 g, 2.40 mmol) and LiOH (0.115 g, 4.81 mmol) afforded Lithium salt of Example 968F (800 mg, 83%) as an off-white solid. MS: [M−H]⁺=400.2.

### Example 968G. tert-Butyl (1R,3s,5S)-3-((2-(((R)-1-(4-amino-7-(1-(2,2-difluoroethyl)-5-fluoro-6-oxo-1,6-dihydropyridin-3-yl)pyrrolo[2,1-f][1,2,4]triazin-5-yl)piperidin-3-yl)carbamoyl)-5-chlorothiophen-3-yl)oxy)-9-azabicyclo[3.3.1]nonane-9-carboxylate

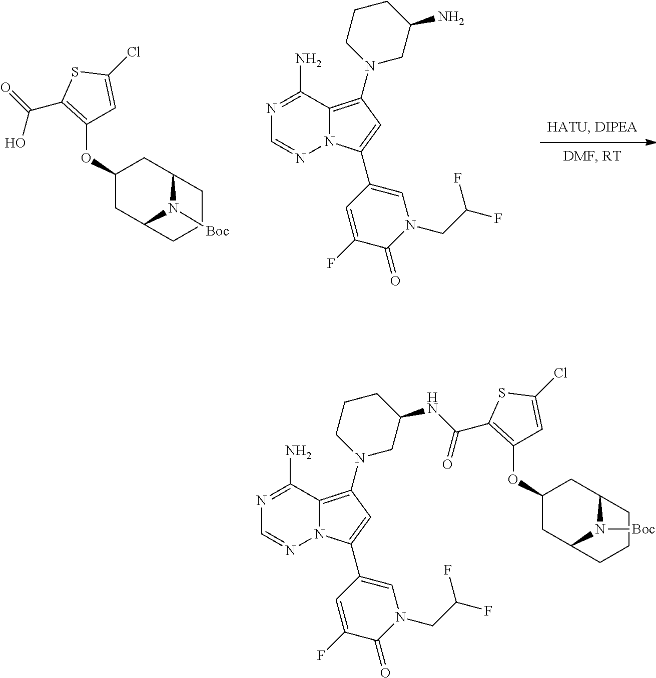

Analogous to the preparation of Example 946, reaction of Example 968F (0.15 g, 0.373 mmol), Example 968D (0.152 g, 0.373 mmol), HATU (0.142 g, 0.373 mmol) and DIEA (0.065 mL, 0.373 mmol) afforded Example 968G (0.21 g, 71%) as a brown solid. MS: [M+H]⁺=791.5.

### Example 968H. 3-(((1R,3s,5S)-9-Azabicyclo[3.3.1]nonan-3-yl)oxy)-N—((R)-1-(4-amino-7-(1-(2,2-difluoroethyl)-5-fluoro-6-oxo-1,6-dihydropyridin-3-yl)pyrrolo[2,1-f][1,2,4]triazin-5-yl)piperidin-3-yl)-5-chlorothiophene-2-carboxamide

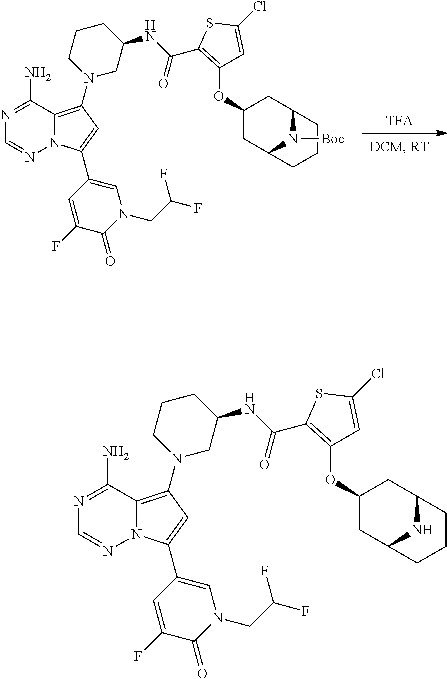

Analogous to the preparation of Example 946C, reaction of Example 968G (0.20 g, 0.253 mmol) and TFA (0.195 mL, 2.53 mmol) afforded Example 968H (15.3 mg, 8%). LC-MS Method E: RT=1.787 min, [M+H]⁺=691.1; 1H NMR (400 MHz, DMSO-d6) δ ppm 8.44 (s, 1H), 8.15 (dd, J=2.3, 11.8 Hz, 1H), 7.85 (s, 1H), 7.50 (s, 2H), 7.01 (s, 1H), 6.78 (s, 1H), 6.55-6.23 (m, 1H), 5.28-5.15 (m, 1H), 4.55 (dt, J=3.3, 15.0 Hz, 2H), 4.19 (br s, 1H), 3.18 (br s, 1H), 2.93 (br s, 3H), 2.22 (dt, J=6.6, 11.8 Hz, 1H), 1.96-1.50 (m, 12H).

### Example 968

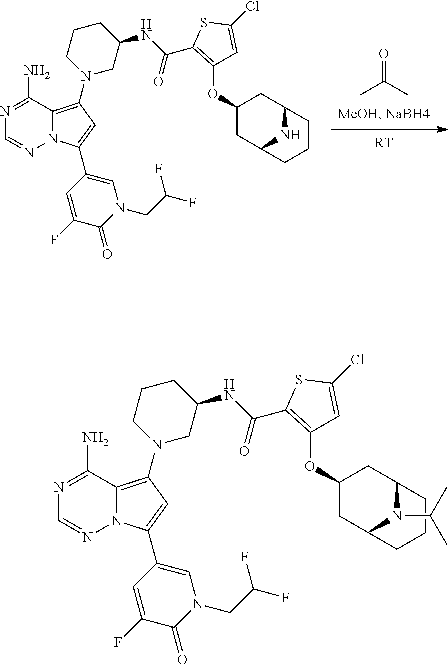

Analogous to the preparation of Example 946D, reaction of Example 968H (0.05 g, 0.072 mmol), acetic acid (2.1 μl, 0.036 mmol), propan-2-one (8.4 mg, 0.145 mmol) and sodium cyanoborohydride (6.8 mg, 0.109 mmol) afforded Example 968 (11.8 mg, 21%). LC-MS Method E: RT=1.762 min, [M+H]⁺=733.2; LC-MS Method F: RT=1.247 min. [M+H]⁺=733.2; ¹H NMR (400 MHz, DMSO-d₆) δ ppm 8.40-8.54 (m, 1H), 8.11-8.23 (m, 1H), 7.81-7.92 (m, 1H), 7.45-7.63 (m, 2H), 6.95-7.08 (m, 1H), 6.61-6.68 (m, 1H), 5.09-5.32 (m, 1H), 4.44-4.67 (m, 2H), 4.14-4.29 (m, 1H), 2.82-3.21 (m, 7H), 1.47-2.02 (m, 14H), 0.64-0.87 (m, 6H).

### Example 969

### Preparation of N-[(3R)-1-{4-amino-7-[6-(propane-2-sulfonyl)pyridin-3-yl]pyrrolo[2,1-f][1,2,4]triazin-5-yl}piperidin-3-yl]-5-chloro-3-{[(R,3R,5S)-8-methyl-8-azabicyclo[3.2.1]octan-3-yl]oxy}thiophene-2-carboxamide

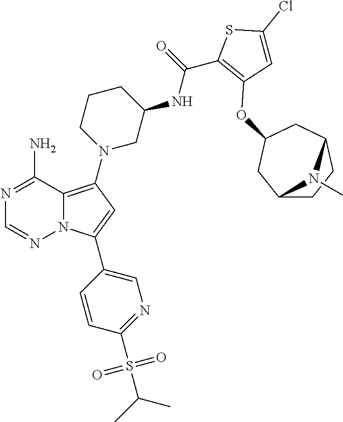 [SMILES: CC(C)S(=O)(=O)c1ccc(-c2cc(N3CCC[C@@H](NC(=O)c4sc(Cl)cc4O[C@@H]4C[C@H]5CC[C@@H](C4)N5C)C3)c3c(N)ncnn23)cn1]

### Example 969A. 5-Bromo-2-(isopropylthio)pyridine

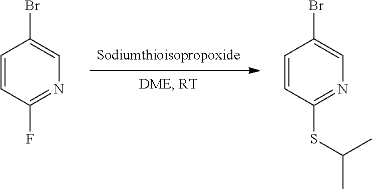

To a solution of 5-bromo-2-fluoropyridine (2.0 g, 11.4 mmol) in DME (40 mL) was added sodium propane-2-thiolate (2.23 g, 22.7 mmol) at rt and the reaction mixture was stirred at rt for 16 h. Then, it was diluted with water (50 mL) and the product was extracted with ethyl acetate (2×50 mL). The combined organic layer was dried over anhydrous sodium sulphate, filtered and concentrated to afford Example 969A (1.8 g, 68%) as an off-white solid. MS: [M+H]⁺=231.9.

### Example 969B. 5-Bromo-2-(isopropylsulfonyl)pyridine

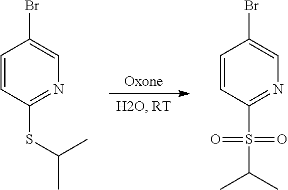

To a solution of Example 969A (2.0 g, 8.62 mmol) in DME (40 mL) was added oxone (15.89 g, 25.8 mmol) at rt and the mixture was stirred at rt for 16 h. Water (50 mL) was added to the reaction mixture and the solid precipitated out was filtered which afforded Example 969B (1.5 g, 66%) as an off-white solid. MS: [M+H]⁺=264.1.

### Example 969C. 2-(Isopropylsulfonyl)-4-(4,4,5,5-tetramethyl-1,3,2-dioxaborolan-2-yl)pyridine

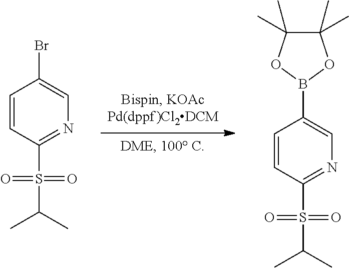

To a solution of Example 969B (0.40 g, 1.51 mmol) in DME (20 mL) was added bis(pinacolato)diboron (0.461 g, 1.82 mmol), potassium acetate (0.223 g, 2.27 mmol) and PdCl₂(dppf)-CH₂Cl₂ adduct (0.062 g, 0.076 mmol) at rt and the mixture was heated at 95° C. for 2 h. The reaction was cooled to it, and water (50 mL) was added followed by ethyl acetate (100 mL). The organic layer was collected, dried over anhydrous sodium sulphate, filtered and concentrated to afford Example 969C (450 mg, 95%). MS: [M+H]⁺=230.1.

### Example 969D. tert-Butyl (R)-(1-(4-amino-7-(6-(isopropylsulfonyl)pyridin-3-yl)pyrrolo[2,1-f][1,2,4]triazin-5-yl)piperidin-3-yl)carbamate

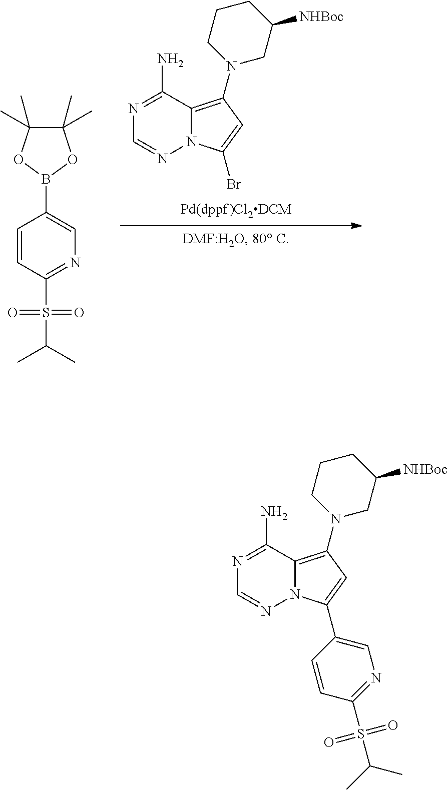

Analogous to the preparation of Example 947, reaction of Intermediate 29 (0.500 g, 1.22 mmol), Example 969C (0.378 g, 1.22 mmol), K₂CO₃ (0.336 g, 2.43 mmol) and PdCl₂(dppf)-CH₂Cl₂ adduct (0.050 g, 0.061 mmol) afforded 0.75 g of Example 969D as a brown gummy solid. MS: [M+H]⁺=516.1.

### Example 969E. (R)-5-(3-Aminopiperidin-1-yl)-7-(6-(isopropylsulfonyl)pyridin-3-yl)pyrrolo[2,1-f][1,2,4]triazin-4-amine

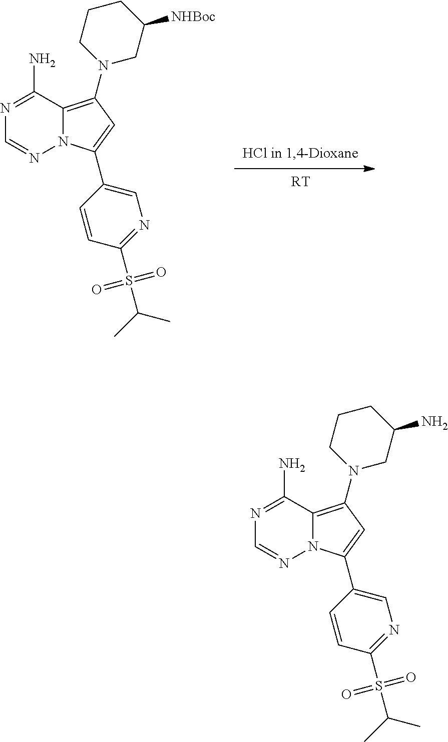 [SMILES: CC(C)S(=O)(=O)c1ccc(-c2cc(N3CCC[C@@H](NC(=O)OC(C)(C)C)C3)c3c(N)ncnn23)cn1 ; "RT" ; "HCl in 1,4-Dioxane" ; CC(C)S(=O)(=O)c1ccc(-c2cc(N3CCC[C@@H](N)C3)c3c(N)ncnn23)cn1]

To a solution of Example 969D (500 mg, 0.970 mmol) in DCM (10 mL) was added HCl in 1,4-dioxane (2.42 mL, 9.70 mmol) at rt and the reaction mixture was stirred at rt for 4 h. Upon concentration, 0.35 g of Example 969E was isolated as a brown gummy solid. MS: [M+H]⁺=416.1.

### Example 969F. tert-butyl (1R,3s,5S)-3-((2-(((R)-1-(4-amino-7-(6-(isopropylsulfonyl)pyridin-3-yl)pyrrolo[2,1-f][1,2,4]triazin-5-yl)piperidin-3-yl)carbamoyl)-5-chlorothiophen-3-yl)oxy)-8-azabicyclo[3.2.1]octane-8-carboxylate

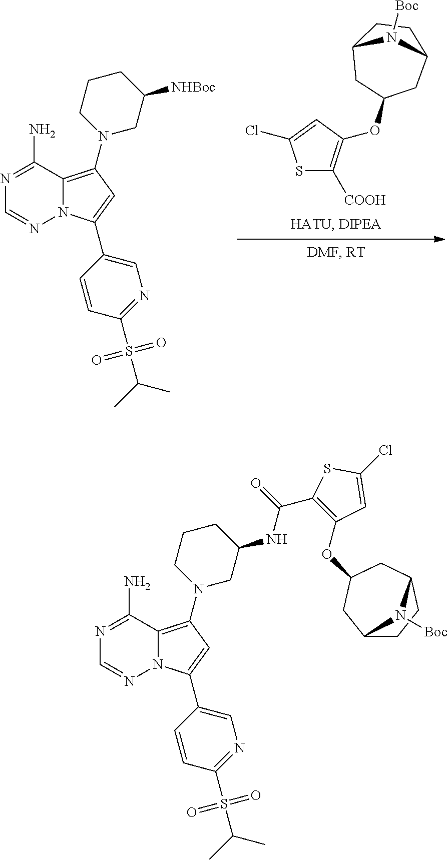

Analogous to the preparation of Example 946, reaction of Example 969E (0.20 g, 0.481 mmol), 3-(((1R,3s,5S)-8-(tert-butoxycarbonyl)-8-azabicyclo[3.2.1]octan-3-yl)oxy)-5-chlorothiophene-2-carboxylic acid (0.266 g, 0.722 mmol), BOP (0.532 g, 1.20 mmol) and DIEA (0.252 mL, 1.44 mmol) afforded Example 969F. MS: [M+H]⁺=785.2.

### Example 969G. N-[(3R)-1-{4-Amino-7-[6-(propane-2-sulfonyl)pyridin-3-yl]pyrrolo[2,1-f][1,2,4]triazin-5-yl}piperidin-3-yl]-3-[(1R,3R,5S)-8-azabicyclo[3.2.1]octan-3-yloxy]-5-chlorothiophene-2-carboxamide

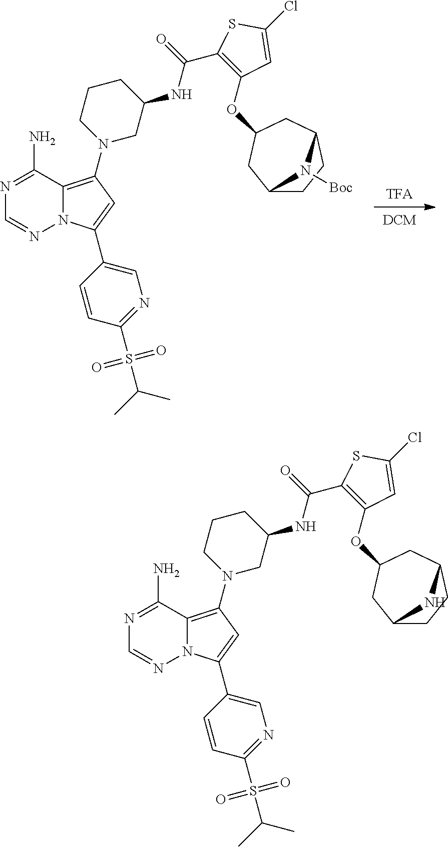

To a stirred solution of Example 969F (0.200 g, 0.255 mmol) in DCM (5 mL) was added TFA (0.196 mL, 2.55 mmol) at it and the reaction mixture was stirred at rt for 12 h. Then, it was concentrated to afford Example 969C (0.15 g, 83%). MS: [M+H]⁺=685.3

### Example 969

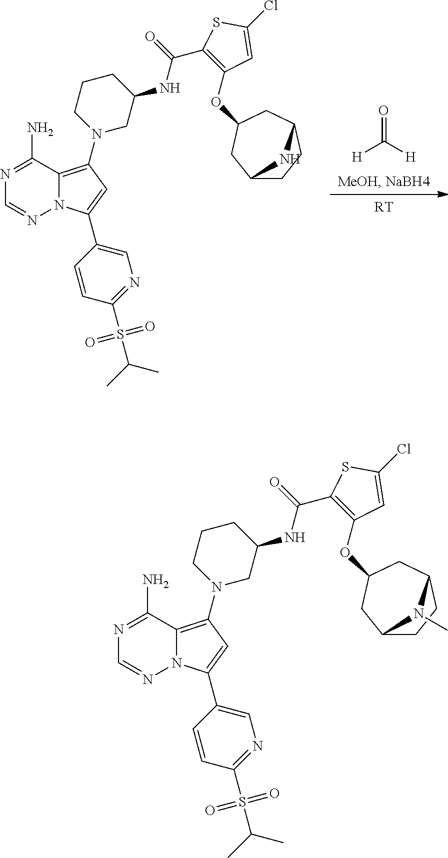

To a stirred solution of Example 969G (0.05 g, 0.073 mmol) in methanol (2 mL) was added acetic acid (4.2 μl, 0.073 mmol), formaldehyde (2.2 mg, 0.073 mmol) and sodium borohydride (2.8 mg, 0.073 mmol) at 0° C. and the reaction mixture was stirred at rt for 2 h. The reaction mixture was diluted with water (20 mL) and extracted with ethyl acetate (220 mL). The combined organic layers were dried over sodium sulfate and evaporated under reduced pressure to afford crude product which was purified by preparative HPLC to obtain Example 969 (10.9 mg, 21%). LC-MS Method E: RT=1.60 min, [M+H]⁺=699.2; LC-MS Method F: RT=1.22 min, [M+H]⁺=699.2; ¹H NMR (400 MHz, DMSO-d₆) δ ppm 9.48-9.36 (m, 1H), 8.89 (dd, J=2.1, 8.4 Hz, 1H), 8.12 (d, J=8.5 Hz, 1H), 7.96 (s, 1H), 7.49 (s, 1H), 7.41 (s, 1H), 4.72-4.68 (m, 1H), 4.20 (br s, 1H), 3.76-3.69 (m, 1H), 3.46 (br d, J=2.0 Hz, 4H), 3.22-3.16 (m, 2H), 3.05-2.79 (m, 1H), 2.63 (br dd, J=1.3, 3.0 Hz, 2H), 2.31-2.14 (m, 2H), 1.91 (s, 10H), 1.22 (d, J=7.0 Hz, 6H).

### Example 970

### Preparation of N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1R,3s,5S)-9-ethyl-9-azabicyclo[3.3.1]nonan-3-yl)oxy)-2-methylthiazole-5-carboxamide

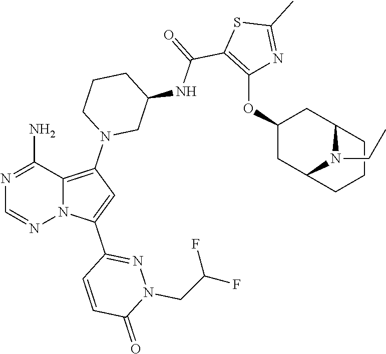 [SMILES: CCN1[C@@H]2CCC[C@H]1C[C@H](Oc1nc(C)sc1C(=O)N[C@@H]1CCCN(c3cc(-c4ccc(=O)n(CC(F)F)n4)n4ncnc(N)c34)C1)C2]

### Example 970A. Ethyl 4-(((1R,3s,5S)-9-(tert-butoxycarbonyl)-9-azabicyclo[3.3.1]nonan-3-yl)oxy)-2-methylthiazole-5-carboxylate

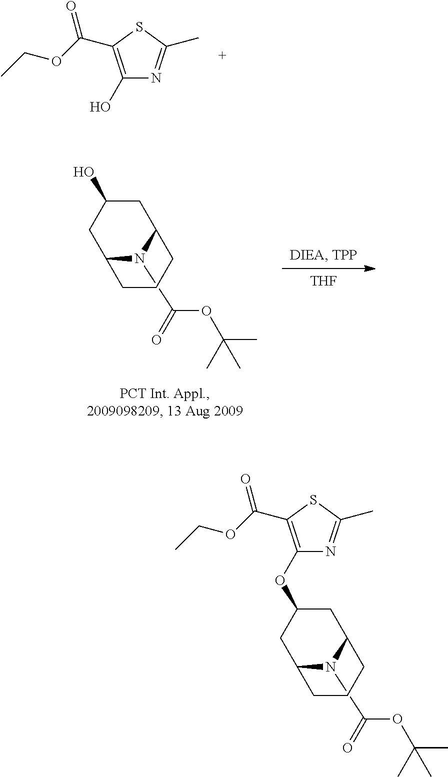 [SMILES: CCOC(=O)c1sc(C)nc1O ; CC(C)(C)OC(=O)N1[C@@H]2CCC[C@H]1C[C@H](O)C2 ; "DIEA, TPP" ; CCOC(=O)c1sc(C)nc1O[C@@H]1C[C@H]2CCC[C@@H](C1)N2C(=O)OC(C)(C)C ; "THF"]

To a solution of tert-butyl 3-hydroxy-9-azabicyclo[3.3.1]nonane-9-carboxylate (1.0 g, 4.14 mmol) and ethyl 4-hydroxy-2-methylthiazole-5-carboxylate (0.776 g, 4.14 mmol) in THF (20 mL) was added triphenylphosphine (1.30 g, 4.97 mmol) and DIAD (0.967 mL, 4.97 mmol) at rt and the reaction mixture was stirred at rt for 12 h. The reaction mixture was concentrated to get crude residue which was purified by flash chromatography (28% ethyl acetate in pet ether) to get Example 970A (1.3 g, 76%) as an off-white solid. MS: [M−H]⁺=411.2.

### Example 970B. Ethyl 4-(((1R,3s,5S)-9-(tert-butoxycarbonyl)-9-azabicyclo[3.3.1]nonan-3-yl)oxy)-2-methylthiazole-carboxylic acid

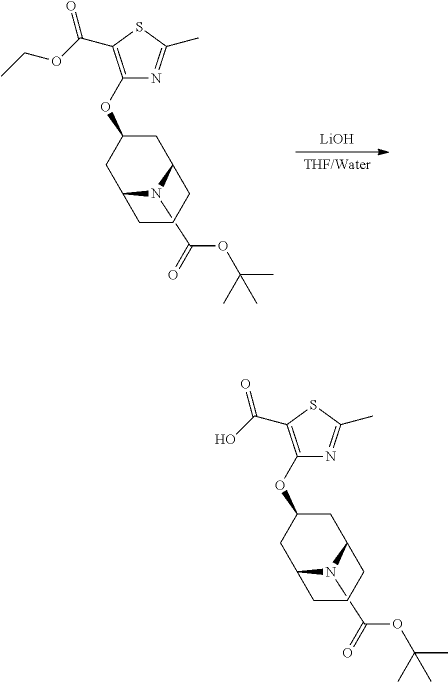 [SMILES: CCOC(=O)c1sc(C)nc1O[C@@H]1C[C@H]2CCC[C@@H](C1)N2C(=O)OC(C)(C)C ; "LiOH" ; "THF/Water" ; Cc1nc(O[C@@H]2C[C@H]3CCC[C@@H](C2)N3C(=O)OC(C)(C)C)c(C(=O)O)s1]

To a stirred solution of Example 970A (1.0 g, 2.52 mmol) in THF (10 mL), methanol (10 mL) and water (5 mL) was added LiOH (0.121 g, 5.04 mmol), stirred at rt for 12 h and concentrated to get Example 970B (800 mg, 83%). MS: [M−H]⁺=381.2.

### Example 970C. 6-Chloro-2-(2,2-difluoroethyl)pyridazin-3(2H)-one

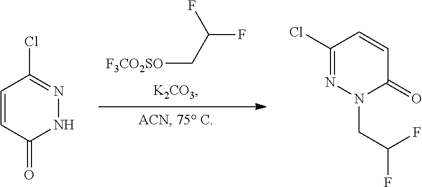

To a stirred solution of 6-chloropyridazin-3(2H)-one (1.0 g, 7.66 mmol) in acetonitrile (20 mL) was added K₂CO₃ (1.59 g, 11.5 mmol) and 2,2-difluoroethyl trifluoromethanesulfonate (1.64 g, 7.66 mmol) at rt and the reaction mixture was stirred at 80° C. for 16 h. The reaction mixture was diluted with water (20 mL) and extracted with ethyl acetate (2×20 mL). The combined organic layers were dried over sodium sulfate and evaporated under reduced pressure to afford crude product which was purified by using 24 g silica gel column (45% ethyl acetate in pet ether) to afford Example 970C (600 mg, 40%) as an off-white solid. MS: [M+H]⁺=195.0; ¹H NMR (300 MHz, CDCl₃) δ ppm 8.27-8.49 (m, 2H), 9.84-10.34 (m, 1H), 10.86-10.97 (m, 1H), 11.19-11.30 (m, 1H).

### Example 970D. 2-(2,2-Difluoroethyl)-6-(4,4,5,5-tetramethyl-1,3,2-dioxaborolan-2-yl)pyridazin-3(2H)-one

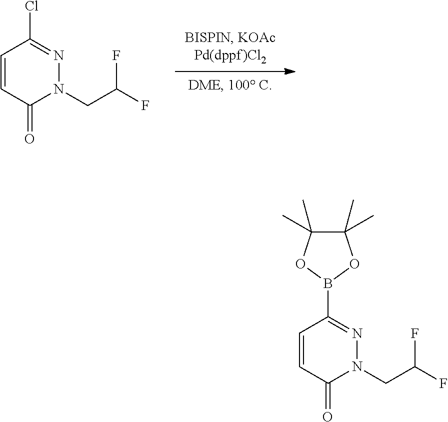 [SMILES: O=c1ccc(Cl)nn1CC(F)F ; "BISPIN, KOAcPd(dppf)Cl2" ; "DME, 100° C." ; CC1(C)OB(c2ccc(=O)n(CC(F)F)n2)OC1(C)C]

To a stirred solution of Example 970C (1.000 g, 5.14 mmol) in DME (20 mL) was added bis(pinacolato)diboron (1.44 g, 5.65 mmol), potassium acetate (0.757 g, 7.71 mmol) and PdCl₂(dppf)-CH₂Cl₂ adduct (0.21 g, 0.257 mmol) at rt and the reaction mixture was heated at 95° C. for 2 h. The mixture was concentrated, then ethyl acetate (100 mL) was added, and the mixture was filtered through Celite. The filtrate was concentrated to afford 1.3 g crude Example 970D. MS: [M+H]⁺=205.1.

### Example 970E. tert-Butyl (R)-(1-(4-amino-7-(1-(2,2-difluoroethyl)-6-oxo-1,6-dihydropyridazin-3-yl)pyrrolo[2,1-f][1,2,4]triazin-5-yl)piperidin-3-yl)carbamate

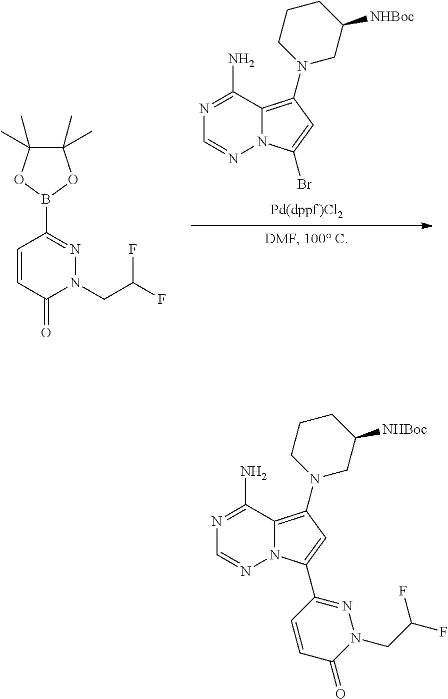

To a solution of Intermediate 29 (0.500 g, 1.22 mmol) and Example 970D (0.522 g, 1.82 mmol) in DMF (10 mL) and water (0.5 mL) was added K₂CO₃ (0.336 g, 2.43 mmol) at rt and the reaction mixture was purged with N₂ for 15 min followed by the addition of PdCl₂(dppf)-CH₂Cl₂ adduct (0.050 g, 0.061 mmol). The reaction mixture was heated at 75° C. for 16 h. The reaction mixture was diluted with water (10 mL) and extracted with ethyl acetate (2×20 mL). The combined organic layers were dried over sodium sulfate and evaporated under reduced pressure to afford crude product which was purified by flash chromatography (75% ethyl acetate in pet ether) to afford Example 970E (0.20 g, 33.5%) as a brown solid. MS: [M+H]⁺=491.4.

### Example 970F. (R)-6-(4-Amino-5-(3-aminopiperidin-1-yl)pyrrolo[2,1-f][1,2,4]triazin-7-yl)-2-(2,2-difluoroethyl)pyridazin-3(2H)-one hydrochloride

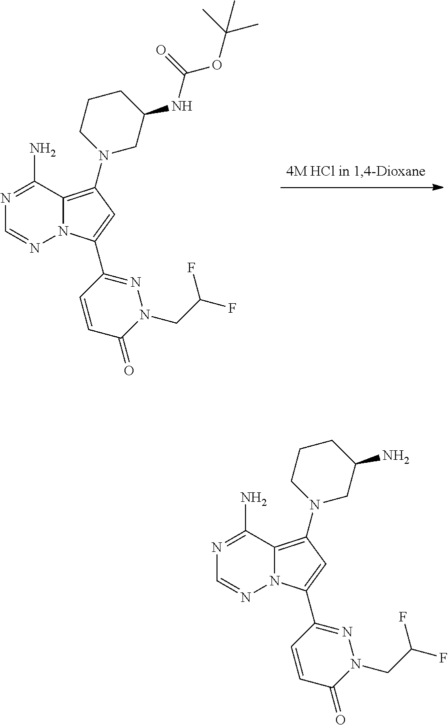 [SMILES: CC(C)(C)OC(=O)N[C@@H]1CCCN(c2cc(-c3ccc(=O)n(CC(F)F)n3)n3ncnc(N)c23)C1 ; "4M HCl in 1,4-Dioxane" ; Nc1ncnn2c(-c3ccc(=O)n(CC(F)F)n3)cc(N3CCC[C@@H](N)C3)c12]

To a solution of Example 970E (0.450 g, 0.917 mmol) in DCM (10 mL) was added HCl in 1,4-dioxane (9.17 mmol) at rt and the reaction mixture was stirred at rt for 16 h. It was concentrated and co-evaporated with toluene (2×50 mL) to get Example 970F (300 mg, 77%) as a brown solid. MS: [M+H]⁺=391.1.

### Example 970G. tert-Butyl (1R,3s,5S)-3-((5-(((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)carbamoyl)-2-methylthiazol-4-yl)oxy)-9-azabicyclo[3.3.1]nonane-9-carboxylate

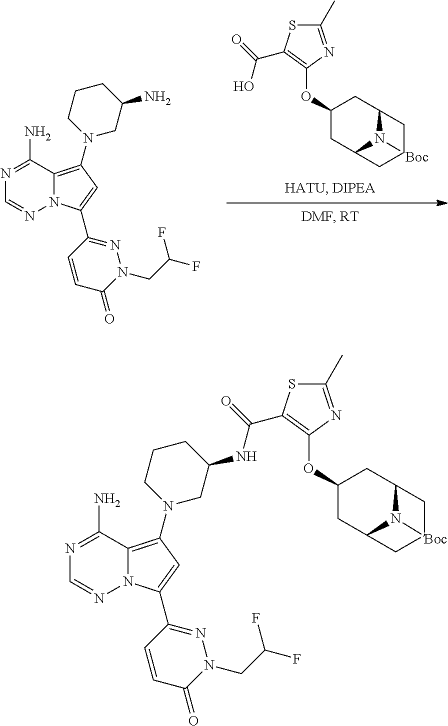

To a solution of Example 970B (0.147 g, 0.384 mmol) in DMF (2 mL) was added Example 970F (0.150 g, 0.384 mmol), BOP (0.170 g, 0.384 mmol) and DIEA (0.067 mL, 0.384 mmol) at rt and the reaction mixture was stirred at rt for 12 h. Water (100 mL) was added, and the product was extracted with ethyl acetate (2×20 mL). The combined organic layer was dried over anhydrous sodium sulphate, filtered and concentrated to afford crude Example 970G (250 mg, 86%) as a brown gummy solid. MS: [M+H]⁺=755.6.

### Example 970H. 4-(((1R,3s,5S)-9-Azabicyclo[3.3.1]nonan-3-yl)oxy)-N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-2-methylthiazole-5-carboxamide

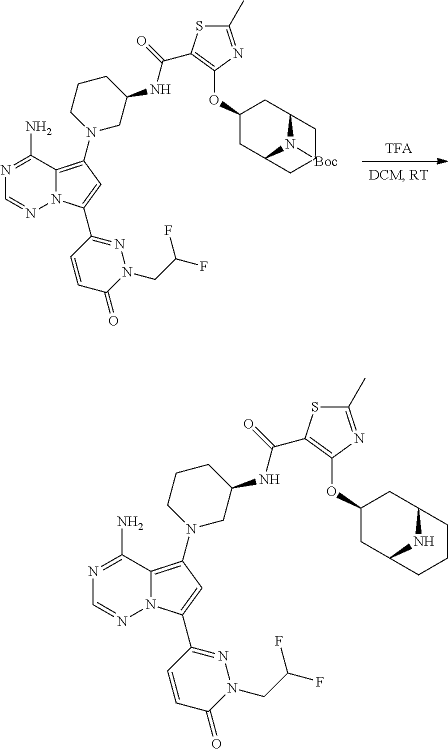

To a solution of Example 970G (0.25 g, 0.331 mmol) in DCM (5 mL) was added TFA (0.26 mL, 3.31 mmol) at rt and the reaction mixture was stirred at rt for 12 h. It was concentrated in vacuo to get crude compound which was purified by preparative HPLC to afford Example 970H (3.4 mg, 1.5%). LC-MS Method E: RT=1.306 min, [M+H]⁺=655.3; LC-MS Method F: RT=1.072 min, [M+H]⁺=655.3; ¹H NMR (400 MHz, DMSO-d₆) δ ppm 8.45 (d, J=9.8 Hz, 1H), 8.23 (br s, 1H), 7.89 (s, 1H), 7.24 (br s, 1H), 7.14 (d, J=10.0 Hz, 1H), 7.03 (s, 1H), 6.88 (s, 1H), 6.66-6.25 (m, 1H), 5.80-5.66 (m, 1H), 4.57 (dt, J=4.4, 14.3 Hz, 2H), 4.24-4.05 (m, 1H), 3.70-3.64 (m, 2H), 3.17 (br d, J=6.0 Hz, 1H), 3.07-2.96 (m, 1H), 2.87 (s, 1H), 2.63 (s, 3H), 2.46-2.36 (m, 2H), 2.06-1.97 (m, 2H), 1.93-1.47 (m, 9H).

### Example 970

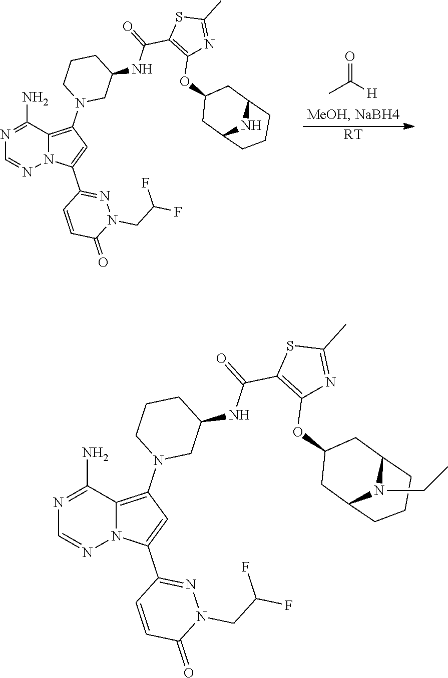

Analogous to the preparation of Example 946D, reaction of Example 970H (0.05 g, 0.076 mmol), acetaldehyde (6.7 mg, 0.15 mmol), acetic acid (0.017 mL, 0.305 mmol) and sodium cyanoborohydride (0.012 g, 0.191 mmol) afforded Example 970 (8 mg). LC-MS Method E: RT=1.472 min, [M+H]⁺=683.3; LC-MS Method F: RT=1.105 min, [M+H]⁺=683.3; ¹H NMR (400 MHz, DMSO-d₆) δ ppm 8.38-8.50 (m, 1H), 8.07-8.35 (m, 1H), 7.85-7.96 (m, 1H), 7.34-7.65 (m, 1H), 7.08-7.19 (m, 1H), 6.94-7.05 (m, 1H), 6.29-6.66 (m, 1H), 5.56-5.79 (m, 1H), 4.46-4.69 (m, 2H), 4.06-4.25 (m, 1H), 2.77-3.30 (m, 7H), 2.56-2.66 (m, 3H), 2.28-2.46 (m, 2H), 1.73-2.08 (m, 9H), 1.34-1.68 (m, 5H), 0.69-1.00 (m, 3H).

### Example 971

### Preparation of N—((R)-1-(4-Amino-7-(1-(2-methoxyethyl)-6-oxo-1,6-dihydropyridazin-3-yl)pyrrolo[2,1-f][1,2,4]triazin-5-yl)piperidin-3-yl)-5-chloro-3-(((1R,3s,5S)-9-methyl-9-azabicyclo[3.3.1]nonan-3-yl)oxy)thiophene-2-carboxamide

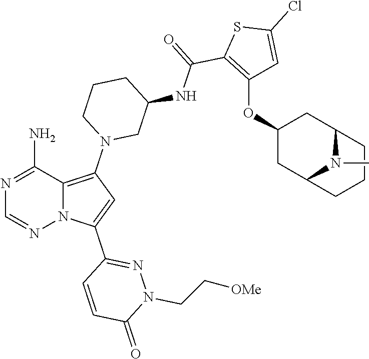 [SMILES: COCCn1nc(-c2cc(N3CCC[C@@H](NC(=O)c4sc(Cl)cc4O[C@@H]4C[C@H]5CCC[C@@H](C4)N5C)C3)c3c(N)ncnn23)ccc1=O]

### Example 971A. 6-Chloro-2-(2-methoxyethyl)pyridazin-3(2H)-one

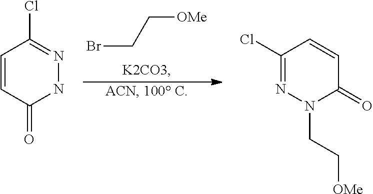

To a solution of 6-chloropyridazin-3(2H)-one (1.0 g, 7.66 mmol) in acetonitrile (20 mL) was added K₂CO₃ (1.59 g, 11.5 mmol) and 1-bromo-2-methoxyethane (1.065 g, 7.66 mmol) at rt and the reaction mixture was stirred at 80° C. for 16 h. The reaction was cooled to rt, concentrated and the residue was dissolved in ethyl acetate (100 mL) and washed with water (1×50 mL) and brine (1×50 mL). Organic layer was collected, dried over magnesium sulfate and concentrated under reduced pressure to get crude product which was purified by flash chromatography (45% ethyl acetate in pet ether) to afford Example 971A (750 mg, 52%) as a brown solid. MS: [M+H]⁺=189.1.

### Example 971B. 2-(2-Methoxyethyl)-6-(4,4,5,5-tetramethyl-1,3,2-dioxaborolan-2-yl)pyridazin-3(2H)-one

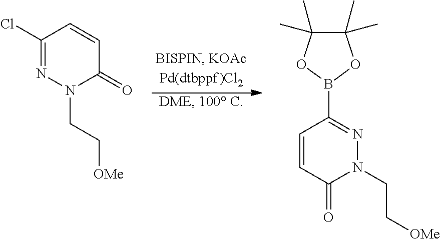

To a solution of Example 971A (0.600 g, 3.18 mmol) in DME (20 mL) was added bis(pinacolato)diboron (0.889 g, 3.50 mmol), potassium acetate (0.468 g, 4.77 mmol) and PdCl₂(dppf)-CH₂Cl₂ adduct (0.130 g, 0.159 mmol) at rt and the reaction mixture was heated at 95° C. for 2 h. The reaction mixture was concentrated and was added ethyl acetate 100 mL and was filtered through Celite. The filtrate was concentrated to afford crude Example 971B (0.8 g) as a brown gummy solid. MS: [M+H]⁺=199.1 (Boronic acid mass).

### Example 971C. tert-Butyl (R)-(1-(4-amino-7-(1-(2-methoxyethyl)-6-oxo-1,6-dihydropyridazin-3-yl)pyrrolo[2,1-f][1,2,4]triazin-5-yl)piperidin-3-yl)carbamate

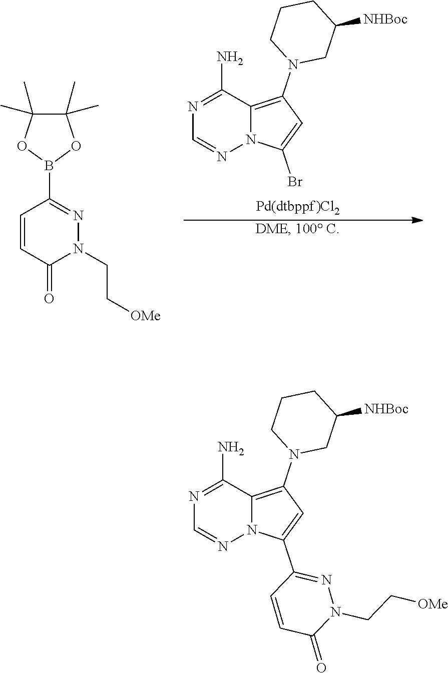 [SMILES: COCCn1nc(B2OC(C)(C)C(C)(C)O2)ccc1=O ; CC(C)(C)OC(=O)N[C@@H]1CCCN(c2cc(Br)n3ncnc(N)c23)C1 ; "DME, 100° C." ; COCCn1nc(-c2cc(N3CCC[C@@H](NC(=O)OC(C)(C)C)C3)c3c(N)ncnn23)ccc1=O]

Analogous to the preparation of Example 970E, reaction of Intermediate 29 (1.00 g, 2.43 mmol) and Example 971B (1.02 g, 3.65 mmol), K₂CO₃ (0.672 g, 4.86 mmol) and PdCl₂(dppf)-CH₂Cl₂ adduct (0.099 g, 0.122 mmol) afforded Example 971C (750 mg, 64%) as a brown solid. MS: [M+H]⁺=485.2.

### Example 971D. (R)-6-(4-Amino-5-(3-aminopiperidin-1-yl)pyrrolo[2,1-f][1,2,4]triazin-7-yl)-2-(2-methoxyethyl)pyridazin-3(2H)-one hydrochloride

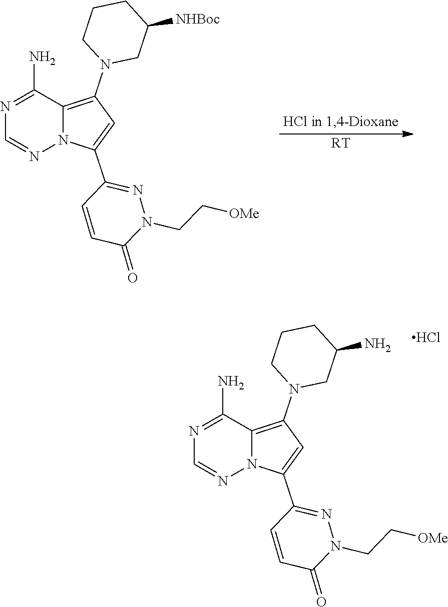 [SMILES: COCCn1nc(-c2cc(N3CCC[C@@H](NC(=O)OC(C)(C)C)C3)c3c(N)ncnn23)ccc1=O ; "RT" ; COCCn1nc(-c2cc(N3CCC[C@@H](N)C3)c3c(N)ncnn23)ccc1=O ; Cl]

Analogous to the preparation of Example 970H, reaction of Example 971C (0.42 g, 0.867 mmol) and HCl in 1,4-dioxane (4M, 8.67 mmol) afforded Example 971D (0.32 g, 88%) as a brown solid. MS: [M+H]⁺=385.3.

### Example 971E. tert-Butyl (1R,3s,5S)-3-((2-(((R)-1-(4-amino-7-(1-(2-methoxyethyl)-6-oxo-1,6-dihydropyridazin-3-yl)pyrrolo[2,1-f][1,2,4]triazin-5-yl)piperidin-3-yl)carbamoyl)-5-chlorothiophen-3-yl)oxy)-9-azabicyclo[3.3.1]nonane-9-carboxylate

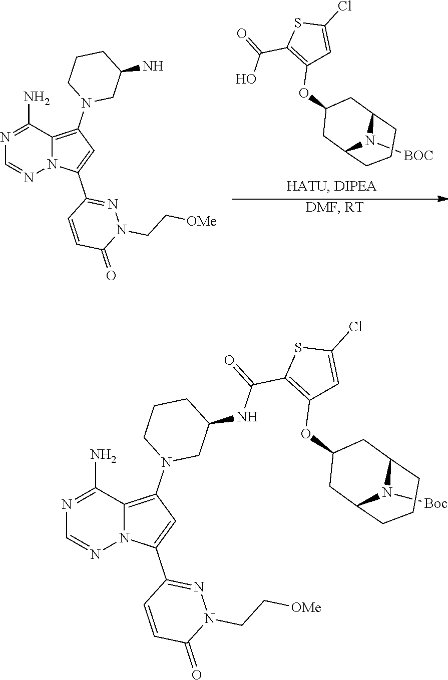

Analogous to the preparation of Example 970G, reaction of Example 971D (0.25 g, 0.650 mmol), 3-(((1R,3s,5S)-9-(tert-butoxycarbonyl)-9-azabicyclo[3.3.1]nonan-3-yl)oxy)-5-chlorothiophene-2-carboxylic acid (0.261 g, 0.650 mmol), BOP (0.431 g, 0.975 mmol) and DIEA (0.284 mL, 1.626 mmol) afforded Example 971E (0.30 g, 60%) as a brown solid. MS: [M+H]⁺=768.3.

### Example 971F. 3-(((1R,3s,5S)-9-Azabicyclo[3.3.1]nonan-3-yl)oxy)-N—((R)-1-(4-amino-7-(1-(2-methoxyethyl)-6-oxo-1,6-dihydropyridazin-3-yl)pyrrolo[2,1-f][1,2,4]triazin-5-yl)piperidin-3-yl)-5-chlorothiophene-2-carboxamide

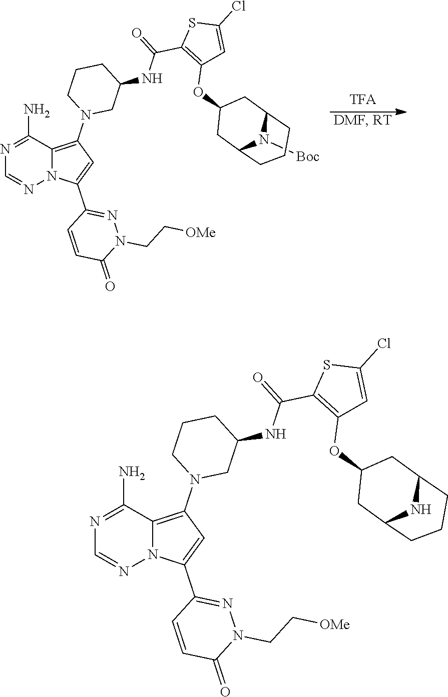

Analogous to the preparation of Example 970H, reaction of Example 971E (0.25 g, 0.325 mmol) and 4M HCl in 1,4-dioxane (0.813 mL, 3.25 mmol) afforded Example 971F (0.19 g, 87%). LC-MS Method E: RT=1.398 min, [M+H]⁺=668.3; ¹H NMR (400 MHz, DMSO-d₆) δ ppm 8.67 (br dd, J=0.8, 2.3 Hz, 1H), 8.62-8.54 (m, 1H), 8.38 (d, J=9.8 Hz, 1H), 8.19 (s, 1H), 7.88 (s, 1H), 7.60 (s, 1H), 7.05 (d, J=9.8 Hz, 1H), 6.99 (s, 1H), 5.31-5.17 (m, 1H), 4.30 (t, J=5.8 Hz, 2H), 4.18 (br s, 1H), 3.82-3.73 (m, 4H), 3.26 (s, 3H), 3.18 (s, 1H), 3.04 (br s, 1H), 2.86 (s, 1H), 2.43 (br dd, J=5.5, 8.3 Hz, 3H), 2.05-1.79 (m, 1H), 1.65 (br s, 2H).

### Example 971

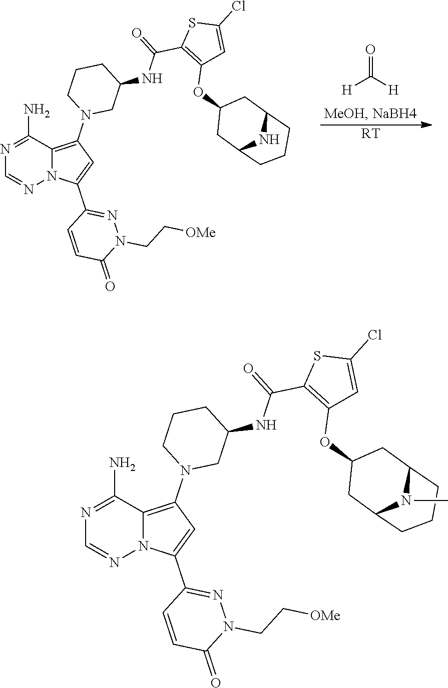

Analogous to the preparation of Example 970, reaction of Example 971F (0.05 g, 0.075 mmol), formaldehyde (0.030 g, 0.374 mmol), acetic acid (0.017 mL, 0.299 mmol) and sodium cyanoborohydride (9.4 mg, 0.150 mmol) afforded Example 971 (11.6 mg, 22%). LC-MS Method E: RT=1.537 min, [M+H]⁺=682.3; LC-MS Method F: RT=1.107 min, [M+H]⁺=682.3; ¹H NMR (400 MHz, DMSO-d₆) δ ppm 8.30-8.53 (m, 1H), 7.80-7.96 (m, 1H), 7.44-7.67 (m, 2H), 7.00-7.12 (m, 1H), 6.95-6.99 (m, 1H), 5.12-5.26 (m, 1H), 4.26-4.34 (m, 2H), 4.14-4.23 (m, 1H), 3.70-3.79 (m, 3H), 3.11-3.25 (m, 4H), 2.63-2.74 (m, 3H), 2.28-2.39 (m, 3H), 2.05-2.09 (m, 1H), 1.73-1.95 (m, 7H), 1.50-1.69 (m, 4H), 1.17-1.31 (m, 1H).

### Example 972

### Preparation of N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-5-chloro-3-(((1R,3s,5S)-9-(2-hydroxyethyl)-9-azabicyclo[3.3.1]nonan-3-yl)oxy)thiophene-2-carboxamide

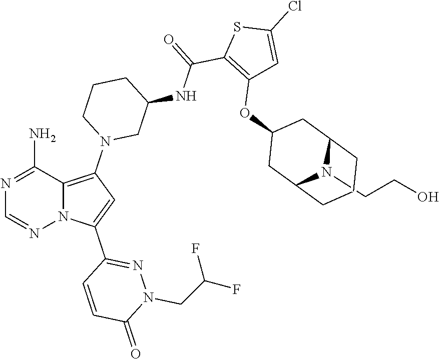 [SMILES: Nc1ncnn2c(-c3ccc(=O)n(CC(F)F)n3)cc(N3CCC[C@@H](NC(=O)c4sc(Cl)cc4O[C@@H]4C[C@H]5CCC[C@@H](C4)N5CCO)C3)c12]

### Example 972A. tert-Butyl (1R,3s,5S)-3-((2-(((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)carbamoyl)-5-chlorothiophen-3-yl)oxy)-9-azabicyclo[3.3.1]nonane-9-carboxylate

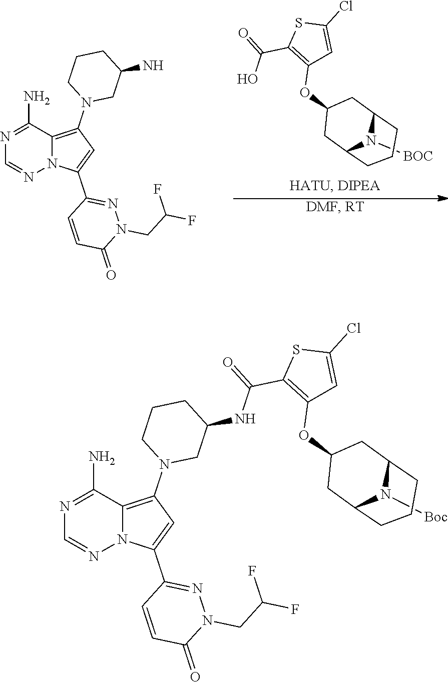

Analogous to the preparation of Example 970G, reaction of 3-(((1R,3s,5S)-9-(tert-butoxycarbonyl)-9-azabicyclo[3.3.1]nonan-3-yl)oxy)-5-chlorothiophene-2-carboxylic acid (0.154 g, 0.384 mmol), (R)-6-(4-amino-5-(3-aminopiperidin-1-yl)pyrrolo[2,1-f][1,2,4]triazin-7-yl)-2-(2,2-difluoroethyl)pyridazin-3(2H)-one (0.150 g, 0.384 mmol), BOP (0.170 g, 0.384 mmol) and DIEA (0.067 mL, 0.384 mmol) afforded Example 972A (250 mg, 84%) as a brown gummy solid. MS: [M+H]⁺=774.5.

### Example 972B. 3-(((1R,3s,5S)-9-Azabicyclo[3.3.1]nonan-3-yl)oxy)-N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-5-chlorothiophene-2-carboxamide

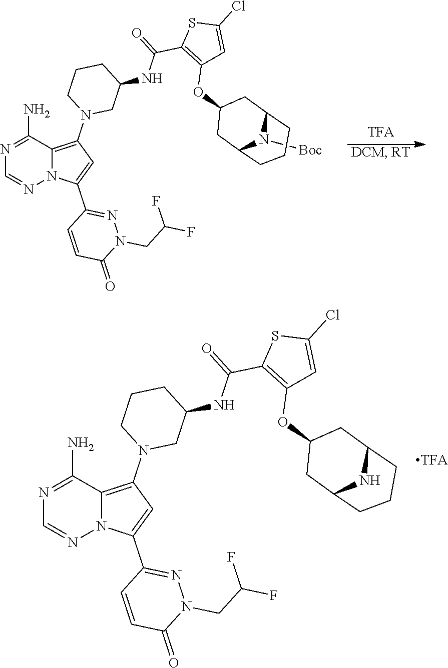

Analogous to the preparation of Example 970H, reaction of Example 972A (0.25 g, 0.323 mmol) and TFA (0.249 mL, 3.23 mmol) afforded Example 972B as TFA salt as a brown gummy solid. MS: [M+H]⁺=674.5.

### Example 972

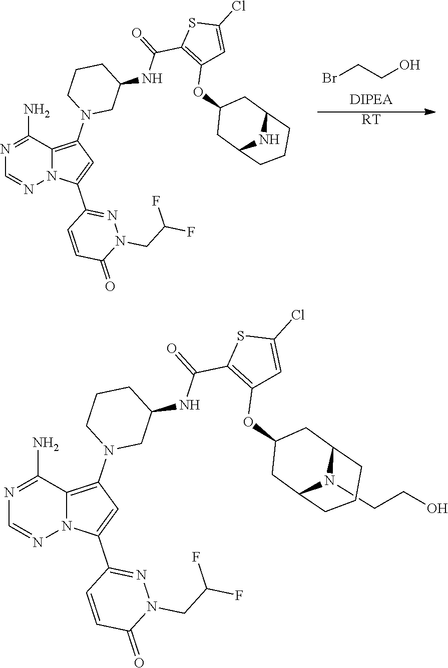

To a solution of Example 972B (0.05 g, 0.074 mmol) in DCM (5 mL) was added DIEA (0.013 ml, 0.074 mmol) and 2-bromoethan-1-ol (9.3 mg, 0.074 mmol) at rt and the reaction mixture was stirred at 60° C. for 12 h. The mixture was concentrated and purified by preparative HPLC to afford Example 972 (16.4 mg, 30%). LC-MS Method E: RT=1.747 min, [M+H]⁺=718.3; LC-MS Method F: RT=1.185 min, [M+H]⁺=718.3; ¹H NMR (400 MHz, DMSO-d₆) δ ppm 8.39-8.50 (m, 1H), 8.08-8.30 (m, 1H), 7.83-7.93 (m, 1H), 7.43-7.60 (m, 2H), 7.09-7.21 (m, 1H), 6.97-7.05 (m, 1H), 6.28-6.65 (m, 1H), 5.12-5.29 (m, 1H), 4.47-4.68 (m, 2H), 4.11-4.28 (m, 1H), 3.54-4.10 (m, 4H), 2.78-3.29 (m, 5H), 2.62-2.75 (m, 2H), 2.31-2.37 (m, 1H), 1.77-2.02 (m, 9H), 1.49-1.65 (m, 3H), 1.20-1.29 (m, 2H).

### Example 973

### Preparation of N—((R)-1-(4-amino-7-(6-oxo-1-(2,2,2-trifluoroethyl)-1,6-dihydropyridazin-3-yl)pyrrolo[2,1-f][1,2,4]triazin-5-yl)piperidin-3-yl)-4-(((1s,4S)-4-(dimethylamino)cyclohexyl)oxy)-2-methylthiazole-5-carboxamide

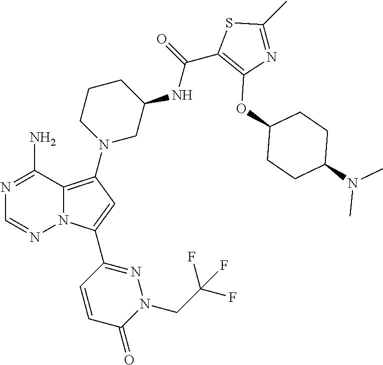 [SMILES: Cc1nc(O[C@H]2CC[C@@H](N(C)C)CC2)c(C(=O)N[C@@H]2CCCN(c3cc(-c4ccc(=O)n(CC(F)(F)F)n4)n4ncnc(N)c34)C2)s1]

### Example 973A. 6-Chloro-2-(2,2,2-trifluoroethyl)pyridazin-3(2H)-one

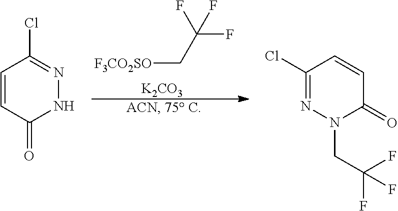

Analogous to the preparation of Example 970C, reaction of 6-chloropyridazin-3(2H)-one (1.0 g, 7.66 mmol), K₂CO₃ (1.59 g, 11.5 mmol) and 2,2,2-trifluoroethyl trifluoromethanesulfonate (1.78 g, 7.66 mmol) at 80° C. afforded Example 973A (1.3 g, 80%) as an off-white solid. MS: [M+H]⁺=213.1.

### Example 973B. 2-(2,2,2-Trifluoroethyl)-6-(trimethylstannyl)pyridazin-3(2H)-one

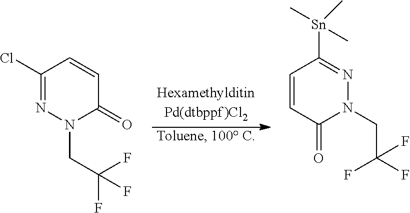

To a stirred solution of Example 973A (650 mg, 3.06 mmol) in toluene (20 mL) was added hexamethylditin (0.70 mL, 3.36 mmol) and [1,1′-Bis(di-tert-butylphosphino)ferrocene]dichloropalladium(II) (199 mg, 0.306 mmol) at it and the reaction mixture was stirred at 100° C. for 2 h. Reaction mixture was filtered through Celite, washed with ethyl acetate and the filtrate was concentrated to afford crude Example 973B (0.85 g, 82%) as a brown gummy solid. MS: [M+H]⁺=343.1.

### Example 973C. tert-Butyl (R)-(1-(4-amino-7-(6-oxo-1-(2,2,2-trifluoroethyl)-1,6-dihydropyridazin-3-yl)pyrrolo[2,1-f][1,2,4]triazin-5-yl)piperidin-3-yl)carbamate

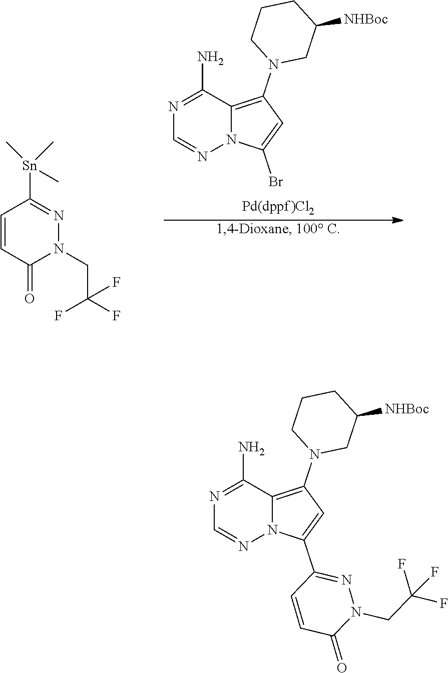 [SMILES: [CH3][Sn]([CH3])([CH3])[c]1ccc(=O)n(CC(F)(F)F)n1 ; CC(C)(C)OC(=O)N[C@@H]1CCCN(c2cc(Br)n3ncnc(N)c23)C1 ; "1,4-Dioxane, 100° C." ; CC(C)(C)OC(=O)N[C@@H]1CCCN(c2cc(-c3ccc(=O)n(CC(F)(F)F)n3)n3ncnc(N)c23)C1]

To a solution of Intermediate 29 (650 mg, 1.58 mmol) and Example 973B (808 mg, 2.37 mmol) in 1,4-dioxane (20 mL) was purged with N₂ for 15 min followed by the addition of [1,1′-Bis(di-tert-butylphosphino)ferrocene]dichloropalladium(II) (129 mg, 0.158 mmol) at rt and the reaction mixture was heated at 105° C. for 18 h. The reaction mixture was filtered through Celite, washed with ethyl acetate (50 mL) and the filtrate was concentrated to get crude residue. Crude residue was suspended in water (50 mL) and extracted with ethyl acetate (2×50 mL). The combined organic layer was dried over anhydrous sodium sulphate, filtered, concentrated, then purified by flash chromatography (80% to 100% ethyl acetate in pet ether) to afford Example 973C (320 mg, 40%) as a brown solid. MS: [M+H]⁺=509.4.

### Example 973D. (R)-6-(4-Amino-5-(3-aminopiperidin-1-yl)pyrrolo[2,1-f][1,2,4]triazin-7-yl)-2-(2,2,2-trifluoroethyl)pyridazin-3(2H)-one hydrochloride

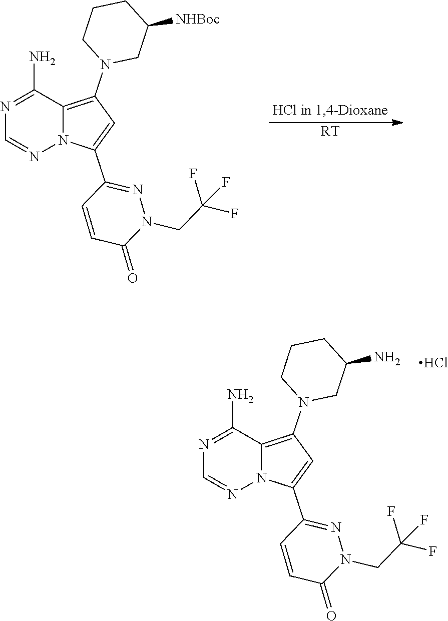 [SMILES: CC(C)(C)OC(=O)N[C@@H]1CCCN(c2cc(-c3ccc(=O)n(CC(F)(F)F)n3)n3ncnc(N)c23)C1 ; "RT" ; Nc1ncnn2c(-c3ccc(=O)n(CC(F)(F)F)n3)cc(N3CCC[C@@H](N)C3)c12 ; Cl]

Analogous to the preparation of Example 970H, reaction of Example 973C (0.5 g, 0.983 mmol) and 4N HCl in 1,4-dioxane (9.83 mmol) afforded Example 973D as HCl salt (0.40 g, 91%) as a brown solid. MS: [M+H]⁺=409.2.

### Example 973E. tert-Butyl ((1S,4s)-4-((5-(((R)-1-(4-amino-7-(6-oxo-1-(2,2,2-trifluoroethyl)-1,6-dihydropyridazin-3-yl)pyrrolo[2,1-f][1,2,4]triazin-5-yl)piperidin-3-yl)carbamoyl)-2-methylthiazol-4-yl)oxy)cyclohexyl)carbamate

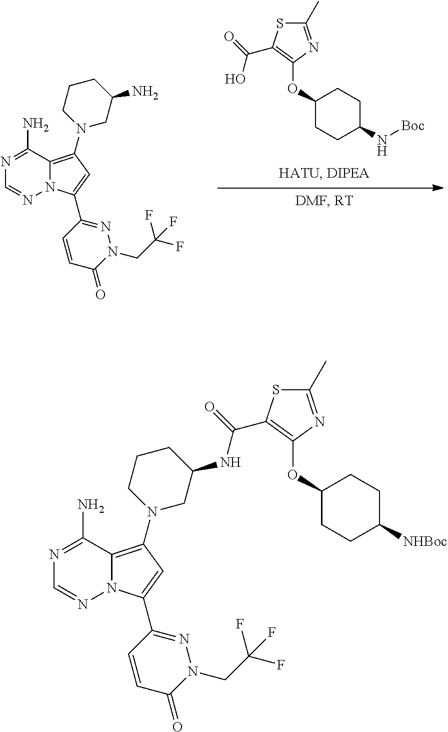

Analogous to the preparation of Example 970G, reaction of 4-(((1s,4s)-4-((tert-butoxycarbonyl)amino)cyclohexyl)oxy)-2-methylthiazole-5-carboxylic acid (0.131 g, 0.367 mmol), Example 973D (0.150 g, 0.367 mmol), BOP (0.162 g, 0.367 mmol) and DIEA (0.064 mL, 0.367 mmol) afforded Example 973E (0.25 g, 91%) as a brown gummy solid. MS: [M−H]⁺=745.5.

### Example 973F. N—((R)-1-(4-Amino-7-(6-oxo-1-(2,2,2-trifluoroethyl)-1,6-dihydropyridazin-3-yl)pyrrolo[2,1-f][1,2,4]triazin-5-yl)piperidin-3-yl)-4-(((1s,4S)-4-aminocyclohexyl)oxy)-2-methylthiazole-5-carboxamide

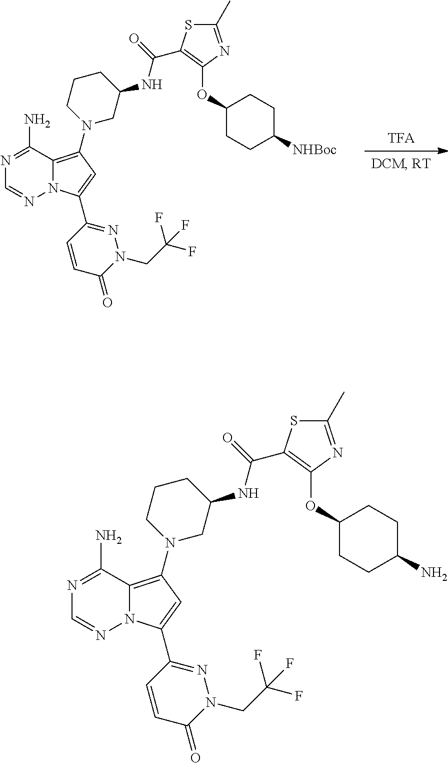

Analogous to the preparation of Example 970H reaction of Example 973E (0.280 g, 0.375 mmol) and TFA (0.289 mL, 3.75 mmol) afforded Example 973F as TFA salt (0.22 g, 91%) as a brown solid. MS: [M+H]⁺=647.3.

### Example 973

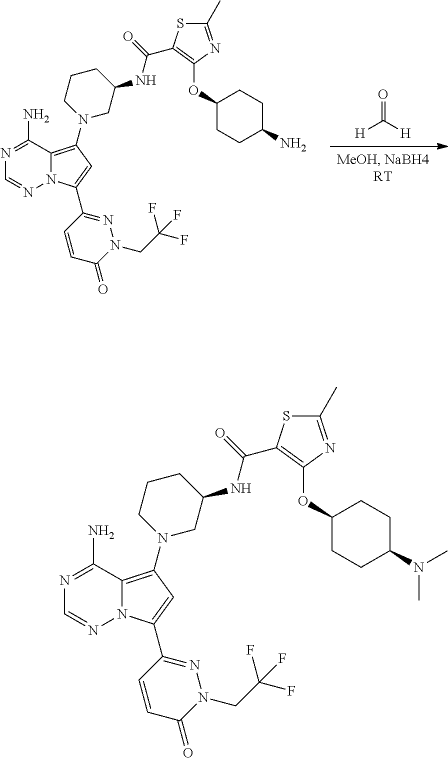 [SMILES: Cc1nc(O[C@H]2CC[C@@H](N)CC2)c(C(=O)N[C@@H]2CCCN(c3cc(-c4ccc(=O)n(CC(F)(F)F)n4)n4ncnc(N)c34)C2)s1 ; C=O ; "MeOH, NaBH4RT" ; Cc1nc(O[C@H]2CC[C@@H](N(C)C)CC2)c(C(=O)N[C@@H]2CCCN(c3cc(-c4ccc(=O)n(CC(F)(F)F)n4)n4ncnc(N)c34)C2)s1]

Analogous to the preparation of Example 969, reaction of Example 973F (0.05 g, 0.077 mmol), acetic acid (8.9 μl, 0.155 mmol) and formaldehyde (5.3 μl, 0.193 mmol) and sodium cyanoborohydride (0.015 g, 0.232 mmol) in MeOH afforded Example 973 (8.9 mg, 17%). LC-MS Method E: RT=1.407 min, [M−H]⁺=675.3; LC-MS Method F: RT=1.140 min, [M−H]⁺=675.3; ¹H NMR (400 MHz, DMSO-d₆) δ ppm 8.39-8.53 (m, 1H), 8.12-8.31 (m, 1H), 7.85-7.95 (m, 1H), 7.12-7.24 (m, 1H), 6.85-6.94 (m, 1H), 5.07-5.14 (m, 1H), 4.91-5.05 (m, 2H), 4.54-4.61 (m, 1H), 4.08-4.22 (m, 1H), 2.73-3.21 (m, 6H), 2.56-2.71 (m, 3H), 2.03-2.39 (m, 6H), 1.44-1.97 (m, 9H), 1.14-1.30 (m, 1H).

### Example 974

### Preparation of N—((R)-1-(4-amino-7-(5-fluoro-1-(2-methoxyethyl)-6-oxo-1,6-dihydropyridin-3-yl)pyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide

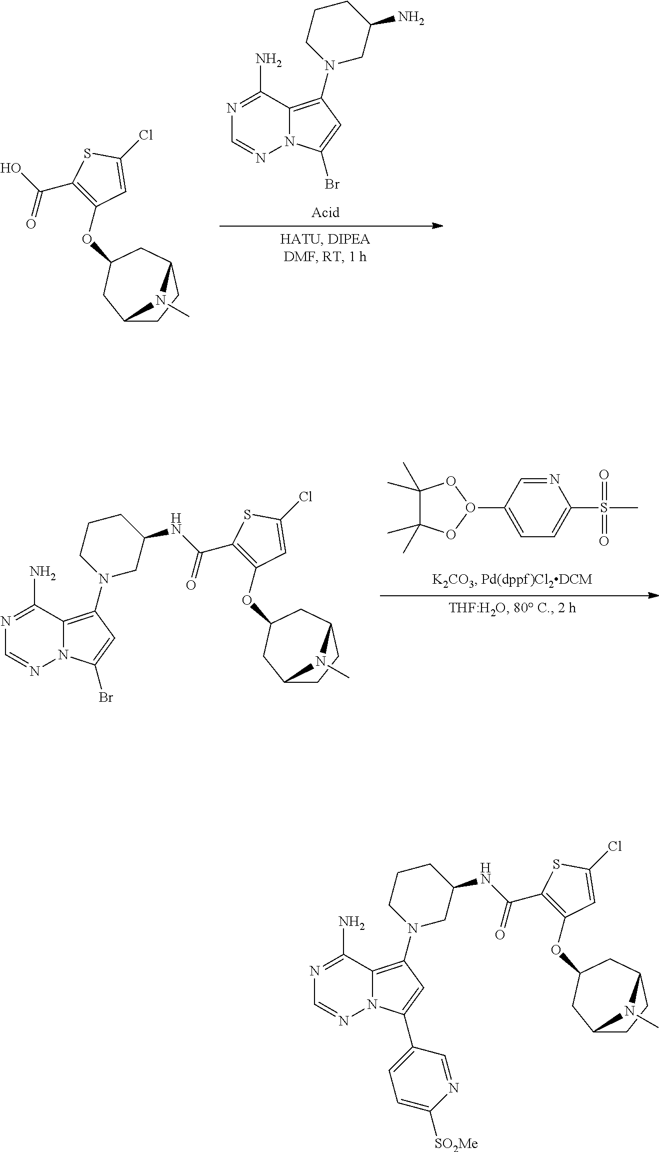

### Example 974A. Preparation of N—((R)-1-(4-amino-7-bromopyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide

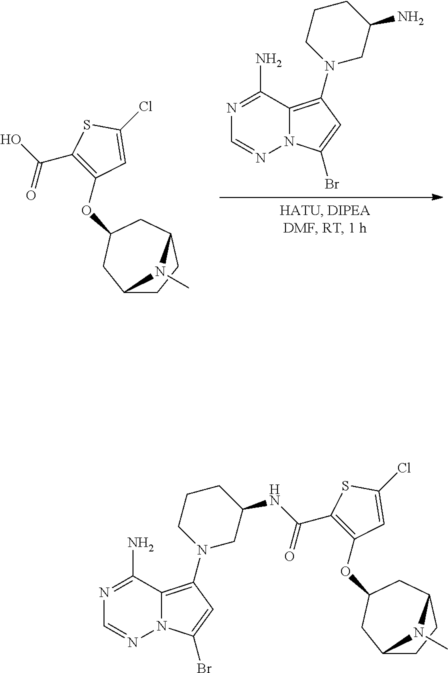

Analogous to the preparation of Example 970G, reaction of Intermediate 60 (0.5 g, 1.66 mmol) and Intermediate 61 (0.58 g, 1.99 mmol) afforded Example 974A (0.7 g, 77%) as a brown solid. LCMS Method E: RT=1.30 min; MS: [M+H]⁺=594.3.

### Example 974

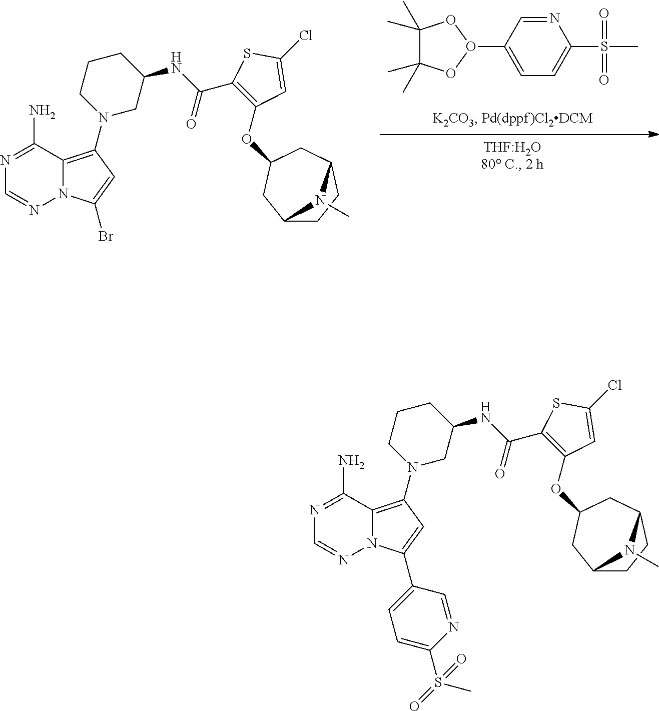

Analogous to the preparation of Example 970E, reaction of Example 974A (200 mg, 0.336 mmol), 2-(methylsulfonyl)-5-(4,4,5,5-tetramethyl-1,3,2-dioxaborolan-2-yl)pyridine (95 mg, 0.34 mmol), K₂CO₃ (0.336 mL, 1.01 mmol) and PdCl₂(dppf)-CH₂C2 adduct (13.7 mg, 0.017 mmol) afforded Example 974 (15 mg, 6.6%). LC-MS Method E: RT=1.473 min, [M−H]⁺=671.2; LC-MS Method F: RT=0.920 min, [M−H]⁺=671.2; ¹H NMR (400 MHz, DMSO-d₆) δ ppm 9.75 (br d, J=1.8 Hz, 1H), 9.41 (d, J=1.8 Hz, 1H), 8.87 (dd, J=2.0, 8.4 Hz, 1H), 8.12 (d, J=8.4 Hz, 2H), 7.95 (s, 1H), 7.57 (s, 1H), 7.38 (s, 1H), 7.29-7.10 (m, 1H), 6.92 (s, 1H), 4.94-4.82 (m, 1H), 4.24-4.15 (m, 1H), 3.97 (br s, 3H), 3.31 (s, 3H), 3.17 (s, 1H), 3.07 (br dd, J=2.4, 10.5 Hz, 1H), 2.97-2.82 (m, 3H), 2.70 (br d, J=4.6 Hz, 6H), 2.39 (br dd, J=3.3, 11.0 Hz, 3H), 2.26-1.69 (m, 2H), 1.52 (br s, 1H).

### Example 975 Preparation of N—((R)-1-(4-amino-7-(1-methyl-1H-pyrazol-5-yl)pyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide

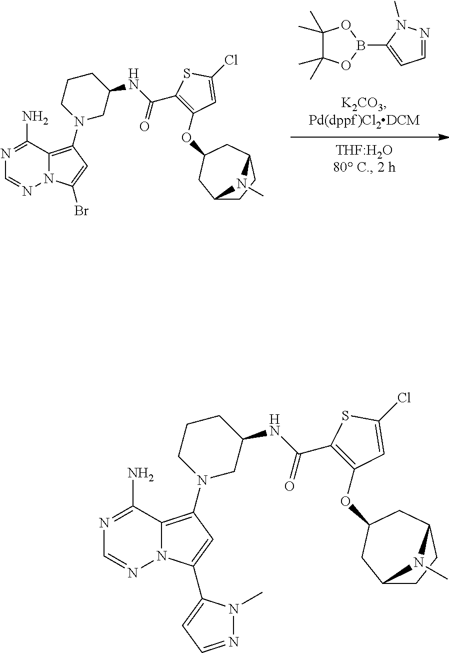 [SMILES: CN1[C@@H]2CC[C@H]1C[C@H](Oc1cc(Cl)sc1C(=O)N[C@@H]1CCCN(c3cc(Br)n4ncnc(N)c34)C1)C2 ; Cn1nccc1B1OC(C)(C)C(C)(C)O1 ; "K2CO3,Pd(dppf)Cl2·DCM" ; "THF:H2O80° C., 2 h" ; CN1[C@@H]2CC[C@H]1C[C@H](Oc1cc(Cl)sc1C(=O)N[C@@H]1CCCN(c3cc(-c4ccnn4C)n4ncnc(N)c34)C1)C2]

Analogous to the preparation of Example 970E, reaction of Example 974A (50 mg, 0.084 mmol). (1-methyl-1H-pyrazol-5-yl)boronic acid (10.6 mg, 0.084 mmol), K₂CO₃ (46.5 mg, 0.336 mmol) and PdCl₂(dppf)-CH₂Cl₂ adduct (3.1 mg, 4.2 mmol) afforded Example 975 (11.4 mg, 22%). LC-MS Method E: RT=1.344 min, [M−H]⁺=596.2; LC-MS Method F: RT=1.005 min, [M−H]⁺=596.1; ¹H NMR (400 MHz, DMSO-d₆) δ ppm 10.01-9.33 (m, 1H), 7.92 (s, 1H), 7.79 (d, J=2.3 Hz, 1H), 7.56 (s, 1H), 7.20-7.13 (m, 1H), 7.00 (d, J=2.3 Hz, 1H), 6.94 (s, 1H), 4.96-4.77 (m, 1H), 4.27-4.12 (m, 1H), 3.97 (br d, J=4.0 Hz, 2H), 3.90 (s, 3H), 3.24-3.19 (m, 2H), 3.11-3.02 (m, 2H), 2.92-2.82 (m, 2H), 2.70 (br d, J=4.5 Hz, 5H), 2.44-2.35 (m, 2H), 2.26-2.11 (m, 5H), 2.06-1.66 (m, 1H).

### Example 976

### Preparation of N—((R)-1-(4-amino-7-(2,5-dihydrofuran-3-yl)-6-fluoropyrrolo[2,1-f][1,2,4]triazin-5-yl)piperidin-3-yl)-5-chloro-3-(((1R,3s,5S)-8-methyl-8-azabicyclo[3.2.1]octan-3-yl)oxy)thiophene-2-carboxamide

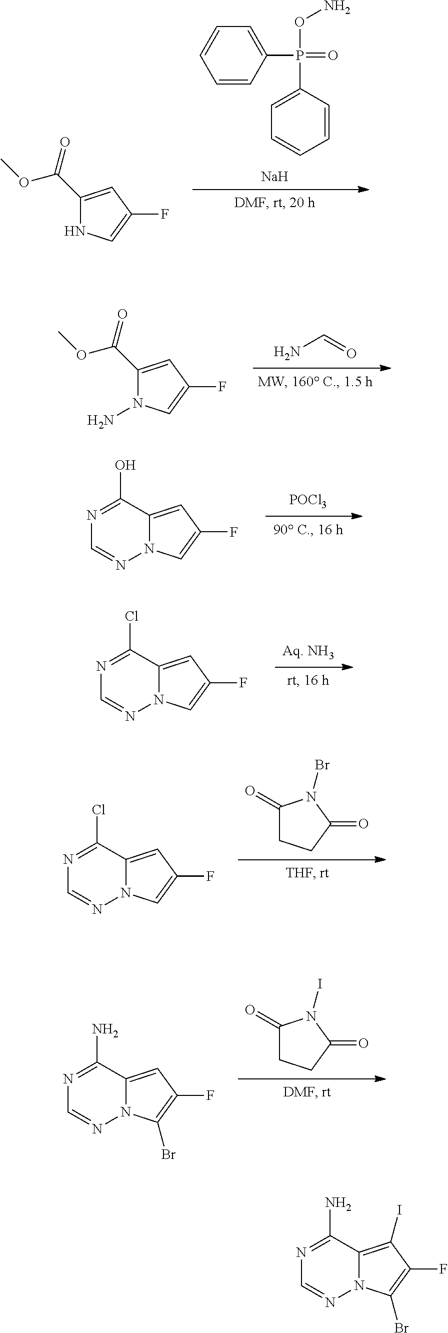

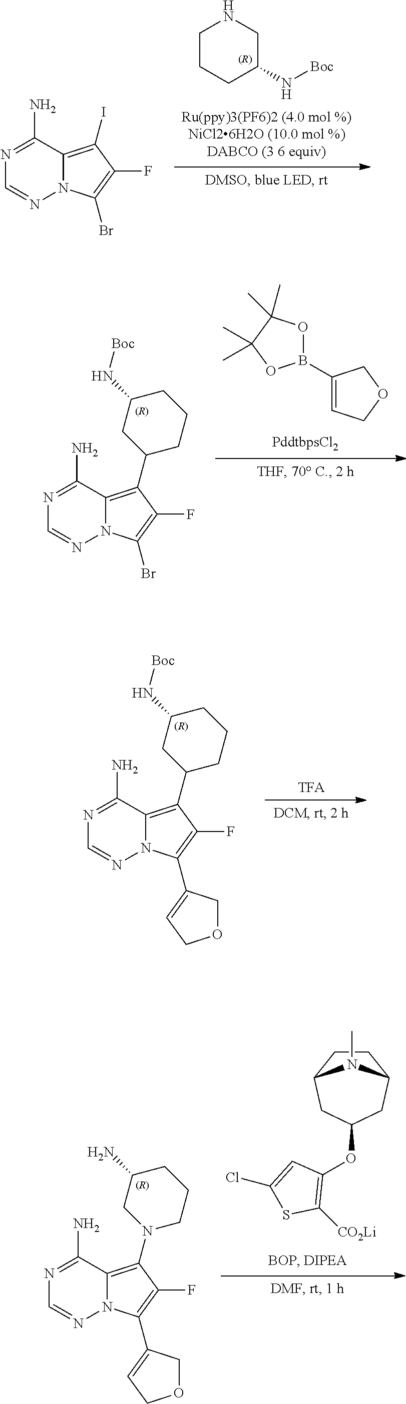

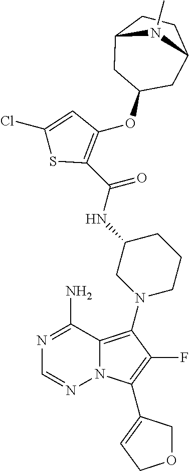 [SMILES: CN1[C@@H]2CC[C@H]1C[C@H](Oc1cc(Cl)sc1C(=O)N[C@@H]1CCCN(c3c(F)c(C4=CCOC4)n4ncnc(N)c34)C1)C2]

### Example 976A. Methyl 1-amino-4-fluoro-1H-pyrrole-2-carboxylate

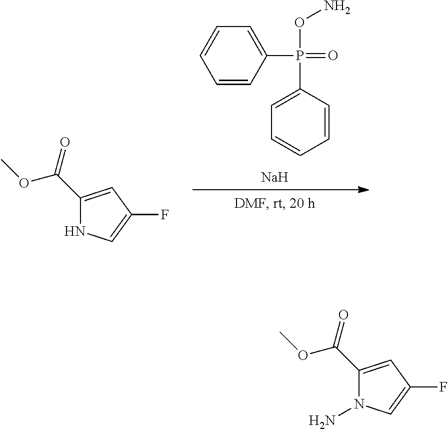

To a stirred suspension of NaH (3.25 g, 81 mmol) in DMF (200 mL) at 0° C. was added the solution of methyl 4-fluoro-1H-pyrrole-2-carboxylate (9.7 g, 67.8 mmol) in DMF (200 mL) and continued stirring for 30 minutes and then added 0-(diphenylphosphinyl)hydroxylamine (28.5 g, 122 mmol) in one portion. The reaction was stirred at rt for 16 h, concentrated and partitioned between DCM and NH₄Cl solution. The organic layer was washed with brine solution and dried over sodium sulphate, filtered, concentrated to afford Example 976A which was used directly to the next step.

### Example 976B. 6-Fluoropyrrolo[2,1-f][1,2,4]triazin-4(3H)-one

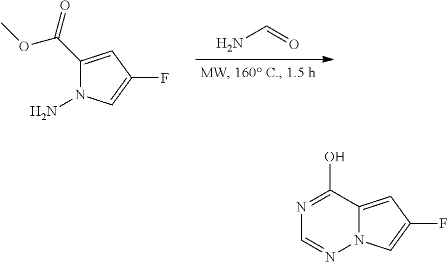

To a well dried microwave vial was added Example 976A (10 g, 58.1 mmol) and formamide (46.3 ml, 1162 mmol) and heated at 160° C. for 1.5 h under microwave irradiation. The reaction mixture was evaporated and partitioned between THF, EtOAc and water. The organic phase was collected, dried over Na₂SO₄, filtered and concentrated. The crude was purified by flash chromatography (0 to 10% MeOH/DCM gradient) to afford Example 976B (3.5 g, 39%) as a brown solid. MS: [M+H]⁺=154.3; ¹H NMR (300 MHz, DMSO-d₆) δ ppm 11.89 (br s, 1H), 8.04-7.86 (m, 1H), 7.79-7.65 (m, 1H), 6.77 (d, J==2.3 Hz, 1H).

### Example 976C. 4-Chloro-6-fluoropyrrolo[2,1-f][1,2,4]triazine

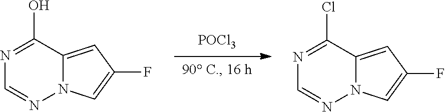 [SMILES: Oc1ncnn2cc(F)cc12 ; "POCl3" ; "90° C., 16 h" ; Fc1cc2c(Cl)ncnn2c1]

A suspension of Example 976B (3.5 g, 22.9 mmol) in POCl₃ (20 mL) was heated at 100° C. for 6 h. The reaction mixture was concentrated in vacuo. It was partitioned between ice water and DCM. The organic phase was separated and washed with water, dried over Na₂SO₄, concentrated under reduced pressure to afford Example 976C (3 g, 76%) as a brown solid. MS: [M+H]⁺=172.3; ¹H NMR (400 MHz, DMSO-d₆) δ ppm 8.54 (s, 1H), 8.45 (dd, J=2.0, 3.0 Hz, 1H), 7.06 (d, J=2.0 Hz, 1H).

### Example 976D. 6-Fluoropyrrolo[2,1-f][1,2,4]triazin-4-amine

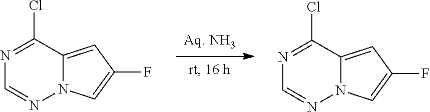 [SMILES: Fc1cc2c(Cl)ncnn2c1 ; "Aq. NH3" ; "rt, 16 h" ; Fc1cc2c(Cl)ncnn2c1]

To a stirred solution of Example 976C (3.0 g, 17.5 mmol) in dioxane (4 mL) was added dropwise 25% aq. ammonia (80 mL) at 10° C. and the reaction mixture was warmed to rt and stirred for 16 h. Reaction was diluted with ice cold water and the precipitated solid was filtered, dried under vacuum to afford Example 976D (2.0 g, 75%). MS: [M+H]⁺=153.0; ¹H NMR (400 MHz, DMSO-d₆) δ ppm 7.85 (s, 1H), 7.73-7.68 (m, 2H), 7.68-7.65 (m, 1H), 6.66 (d, J=2.0 Hz, 1H).

### Example 976E. 7-Bromo-6-fluoropyrrolo[2,1-f][1,2,4]triazin-4-amine

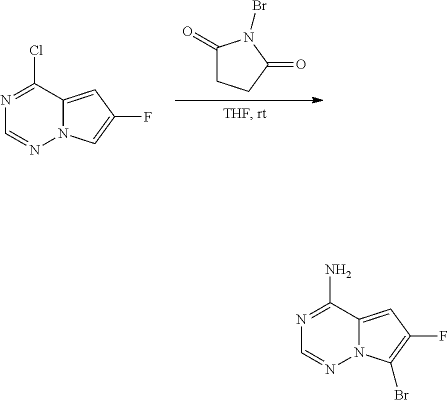

To a stirred solution of Example 976D (1.68 g, 11.0 mmol) in THF (20 mL) was added solution of NBS (1.96 g, 11.0 mmol) in THF (20 mL) at 0° C. and the reaction mixture was stirred at rt for 4 h. Evaporated to dryness and partitioned between EtOAc and water. The organic phase was separated and washed with water, dried over Na₂SO₄, concentrated under reduced pressure. The crude was purified by flash chromatography (0 to 10% MeOH/DCM gradient) to afford Example 976E (1.61 g, 63%) as a yellow solid. MS: [M+H]⁺=231.10; ¹H NMR (400 MHz, DMSO-d₆) δ ppm 7.99 (s, 1H), 7.88 (br s, 2H), 6.89 (s, 1H).

### Example 976F. 7-Bromo-6-fluoro-5-iodopyrrolo[2,1-f][1,2,4]triazin-4-amine

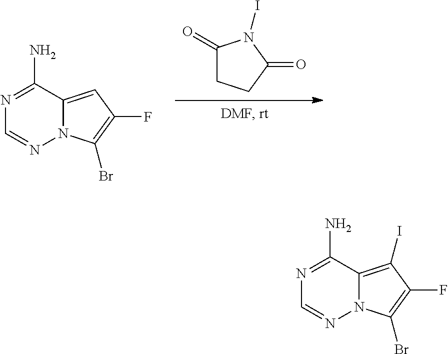

To a stirred solution of Example 976E (1.05 g, 4.54 mmol) in DMF (5 mL) was added NIS (1.02 g, 4.54 mmol) and the reaction mixture was stirred at rt for 16 h and quenched with water (10 mL). The solid precipitate was filtered, washed with water, dried under vacuum to afford Example 976F (1.6 g, 90%) as an off-white solid. MS: [M+H]⁺=356.95; ¹H NMR (400 MHz, DMSO-d₆) δ ppm 8.07 (s, 1H).

### Example 976G. tert-Butyl (R)-(1-(4-amino-7-bromo-6-fluoropyrrolo[2,1-f][1,2,4]triazin-5-yl)piperidin-3-yl)carbamate

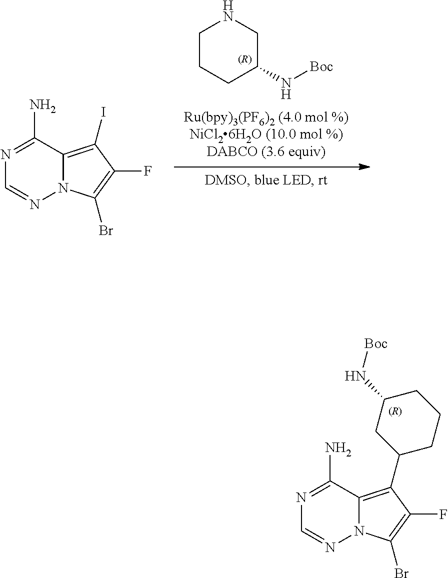

To a mixture of Example 976F (2.15 g, 6.02 mmol), tert-butyl (R)-piperidin-3-ylcarbamate (4.83 g, 24.1 mmol), nickel(II) chloride hexahydrate (0.286 g, 1.21 mmol), tris(2,2′-bipyridine)ruthenium(II) hexafluorophosphate (0.104 g, 0.120 mmol) and DABCO (2.16 g, 19.3 mmol) was added DMSO (30 mL) and the reaction mixture was degassed with N₂, capped and stirred under blue LED irradiation at rt for 16 h. Reaction mixture was partitioned between EtOAc and brine solution. The organic layer was separated, dried over sodium sulphate, concentrated and purified by flash chromatography (0 to 10% MeOH/DCM gradient) to afford Example 976G (200 mg, 4%) as a yellow solid. MS: [M+H]⁺=356.95; ¹H NMR (400 MHz, DMSO-d₆) δ ppm 8.12-7.99 (m, 1H), 7.84 (br s, 2H), 3.19-3.11 (m, 3H), 3.14-2.69 (m, 3H), 1.91-1.51 (m, 4H), 1.35 (s, 9H).

### Example 976H. tert-Butyl (R)-(1-(4-amino-7-(2,5-dihydrofuran-3-yl)-6-fluoropyrrolo[2,1-f][1,2,4]triazin-5-yl)piperidin-3-yl)carbamate

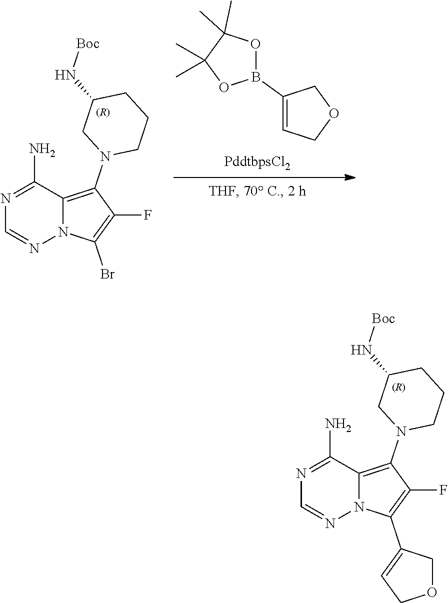

[1,1′-bis(di-tert-butylphosphino)ferrocene]dichloropalladium(II) (8.2 mg, 0.013 mmol) was added to the stirred solution of Example 976G (180 mg, 0.210 mmol), 2-(2,5-dihydrofuran-3-yl)-4,4,5,5-tetramethyl-1,3,2-dioxaborolane (45.2 mg, 0.231 mmol) and aq. potassium phosphate tribasic (2M, 0.21 mL, 0.42 mmol) in THF (3 mL) and the reaction mixture was heated at 70° C. for 2 h. After cooling, the mixture was partitioned between EtOAc and brine solution. The organic layer was separated, dried over sodium sulphate, concentrated and purified by silica flash chromatography (0-80% EtOAc in DCM) to afford Example 976H (40 mg, 19%) as a yellow solid. MS: [M+H]⁺=419.35; ¹H NMR (400 MHz, DMSO-d₆) δ ppm 7.93-7.90 (m, 1H), 6.74-6.71 (m, 1H), 6.70-6.66 (m, 1H), 4.99 (br s, 2H), 4.75-4.68 (m, 2H), 3.17-3.05 (m, 1H), 3.03-2.93 (m, 1H), 1.44 (s, 2H), 1.28-1.18 (m, 4H), 1.05 (s, 9H).

### Example 9761. (R)-5-(3-Aminopiperidin-1-yl)-7-(2,5-dihydrofuran-3-yl)-6-fluoropyrrolo[2,1-f][1,2,4]triazin-4-amine

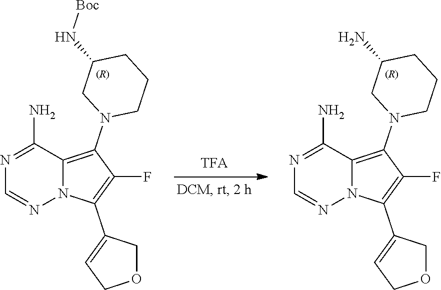

To a stirred solution of Example 976H (40 mg, 0.096 mmol) in DCM (2 mL) was added TFA (0.110 mL, 1.43 mmol) and stirred at rt for 2 h. The residue was co-evaporated with DCM/MeOH to afford Example 9761 (25 mg, 61%) as a yellow solid. MS: [M+H]⁺=319.25; ¹H NMR (400 MHz, DMSO-d₆) δ ppm 7.96 (s, 1H), 6.77-6.68 (m, 1H), 5.09-4.97 (m, 2H), 4.79-4.70 (m, 2H), 3.16-2.82 (m, 4H), 2.12-1.94 (m, 2H), 1.86-1.48 (m, 3H).

### Example 976

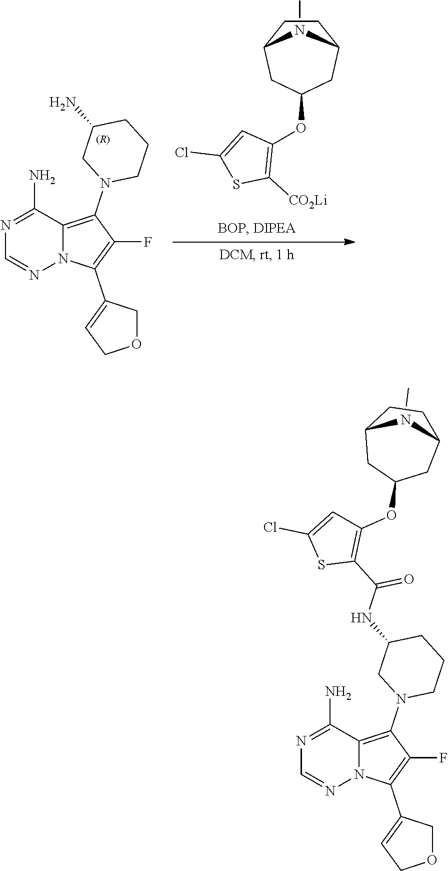

To a stirred solution of Example 9761 (42.2 mg, 0.097 mmol), lithium 5-chloro-3-(((1R,3s,5S)-8-methyl-8-azabicyclo[3.2.1]octan-3-yl)oxy)thiophene-2-carboxylate (30 mg, 0.097 mmol) and BOP (51.7 mg, 0.117 mmol) in DMF (2 mL), was added DIEA (0.051 mL, 0.292 mmol) and the reaction mixture was stirred at RT for 2 h. Mixture was concentrated and purified by preparative HPLC to afford Example 976 (15.4 mg, 25%). LC-MS Method E: RT=1.69 min, [M+H]⁺=602.2; LC-MS Method F: RT=1.21 min, [M+H]⁺=602.2; ¹H NMR (400 MHz, DMSO-d₆) δ ppm 7.94 (s, 1H), 7.48 (s, 1H), 6.71 (t, J=2.1 Hz, 1H), 5.01 (br s, 2H), 4.73 (br s, 3H), 4.22-4.01 (m, 1H), 2.55-2.53 (m, 2H), 2.30-2.24 (m, 2H), 2.08 (s, 3H), 2.01-1.84 (m, 5H), 1.76 (br s, 8H), 1.24 (s, 2H).

### Example 977

### Preparation of N—((R)-1-(4-amino-7-(5-oxo-2,5-dihydrofuran-3-yl)pyrrolo[2,1-f][1,2,4]triazin-5-yl)piperidin-3-yl)-4-(((1s,4S)-4-(isopropylamino)cyclohexyl)oxy)-2-methylthiazole-5-carboxamide

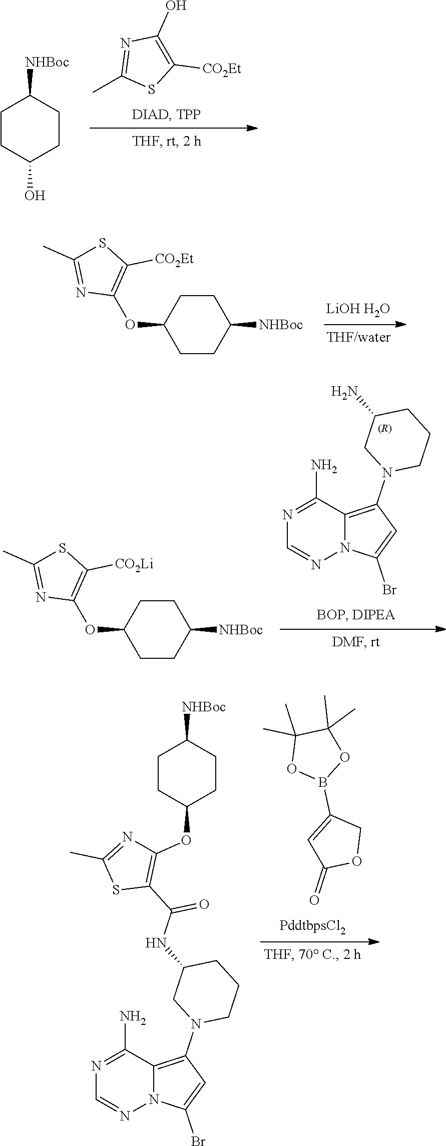

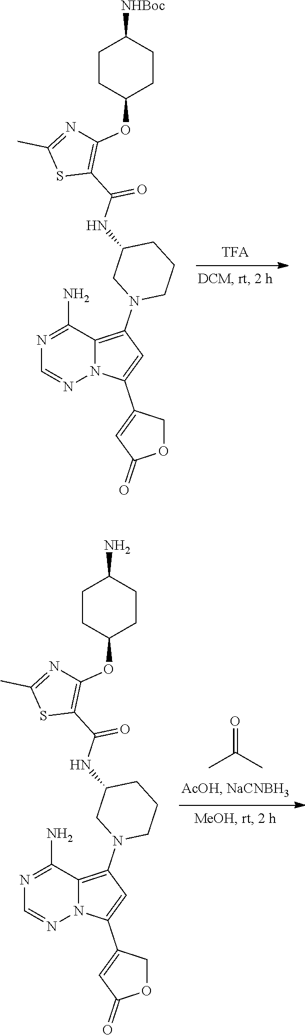

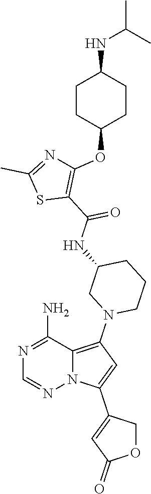 [SMILES: Cc1nc(O[C@H]2CC[C@@H](NC(C)C)CC2)c(C(=O)N[C@@H]2CCCN(c3cc(C4=CC(=O)OC4)n4ncnc(N)c34)C2)s1]

### Example 977A. Ethyl 4-(((1s,4s)-4-((tertbutoxycarbonyl)amino)cyclohexyl)oxy)-2-methylthiazole-5-carboxylate

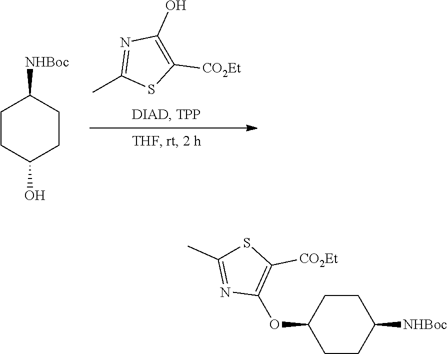

To a stirred solution of ethyl 4-hydroxy-2-methylthiazole-5-carboxylate (10 g, 53.4 mmol), ter-butyl ((1r,4r)-4-hydroxycyclohexyl)carbamate (11.50 g, 53.4 mmol) and triphenylphosphine (21.02 g, 80 mmol) in THF (100 mL) was added DIAD (23.89 mL, 123 mmol) dropwise and the reaction mixture was stirred at RT for 4 h. The mixture was concentrated in vacuo and purified by flash chromatography (0-20% EtOAc in Pet Ether) to afford Example 977A (13.5 g, 65%) as a yellow solid. MS: [M+H]⁺=385.15; ¹H NMR (400 MHz, CHCl₃-d) δ ppm 5.19-5.14 (m, 1H), 4.33-4.26 (m, 2H), 3.63-3.51 (m, 1H), 2.62 (s, 3H), 2.09-2.00 (m, 2H), 1.85-1.77 (m, 2H), 1.75-1.67 (m, 4H), 1.46 (s, 9H), 1.35-1.31 (m, 4H).

### Example 977B. Lithium 4-(((1s,4s)-4-((tertbutoxycarbonyl)amino)cyclohexyl)oxy)-2-methylthiazole-5-carboxylate

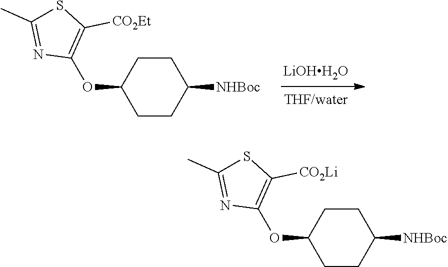

To a stirred solution of Example 977A (6 g, 15.61 mmol) in ethanol (20 mL), 30 mL) and water (10 mL) was added LiOH·H₂O (1.12 g, 46.8 mmol) and stirred at room temperature for 16 h. The mixture was concentrated in vacuo to afford Example 977B (6.7 g, 95%) as a white solid. MS: [M+H]⁺=355.05; ¹H NMR (400 MHz, CDCl₃) δ ppm 5.20-5.05 (m, 1H), 3.91-3.73 (m, 1H), 2.54 (s, 3H), 2.08-1.84 (m, 5H), 1.42 (s, 9H), 1.29 (d, J=6.5 Hz, 3H).

### Example 977C. tert-Butyl ((1S,4s)-4-((5-(((R)-1-(4-amino-7-bromopyrrolo[2,1-f][1,2,4]triazin-5-yl)piperidin-3-yl)carbamoyl)-2-methylthiazol-4-yl)oxy)cyclohexyl)carbamate

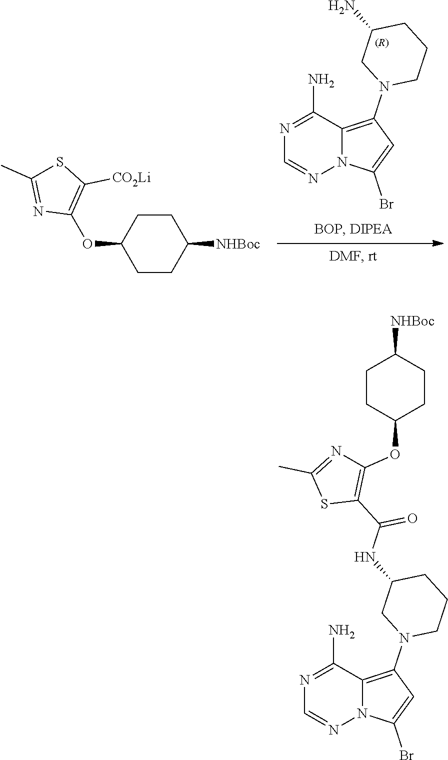

To a stirred solution of Intermediate 61, HCl salt (4.0 g, 11.5 mmol), Example 977B (4.17 g, 11.5 mmol) and BOP (6.11 g, 13.8 mmol) in DMF (30 mL) was added DIEA (10.05 mL, 57.5 mmol) and the reaction mixture was stirred at RT for 4 h. The reaction mixture was partitioned between EtOAc and brine solution. The organic layer was separated, dried over sodium sulphate, concentrated and the residue was purified by flash chromatography (0-20% MeOH in DCM) to afford Example 977C (14.4 g, 82%). MS: [M+H]⁺=651.20; ¹H NMR (400 MHz, DMSO-d₆) δ ppm 7.83 (s, 1H), 7.22-7.06 (m, 1H), 6.76 (s, 2H), 5.09-5.00 (m, 1H), 4.17-4.06 (m, 1H), 3.70-3.53 (m, 1H), 3.22-3.05 (m, 3H), 2.99-2.90 (m, 1H), 2.82-2.69 (m, 2H), 2.62-2.57 (m, 3H), 1.87-1.75 (m, 2H), 1.70 (br s, 6H), 1.38 (s, 9H), 1.29-1.21 (m, 4H).

### Example 977D. 4-(4,4,5,5-Tetramethyl-1,3,2-dioxaborolan-2-yl)furan-2(5H)-one

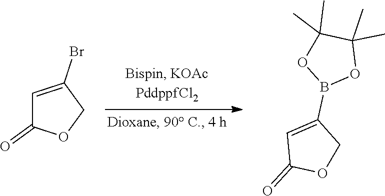

PdCl₂(dppf)-CH₂Cl₂ adduct (120 mg, 0.147 mmol) was added to the stirred solution of 4-bromofuran-2(5H)-one (400 mg, 2.454 mmol), bis(pinacolato)diboron (935 mg, 3.68 mmol) and potassium acetate (361 mg, 3.68 mmol) in dioxane (10 mL) and the reaction mixture was heated at 90° C. for 3 h. The mixture was cooled to RT, diluted with THF, filtered through Celite bed and the filtrate was concentrated in vacuo to afford Example 977D as a brown gummy solid; ¹H NMR (400 MHz, DMSO-d₆) δ ppm 6.39 (s, 1H), 4.95 (d, J=2.0 Hz, 2H), 1.15 (s, 12H).

### Example 977E. tert-Butyl ((1S,4s)-4-((5-(((R)-1-(4-amino-7-(5-oxo-2,5-dihydrofuran-3-yl)pyrrolo[2,1-f][1,2,4]triazin-5-yl)piperidin-3-yl)carbamoyl)-2-methylthiazol-4-yl)oxy)cyclohexyl)carbamate

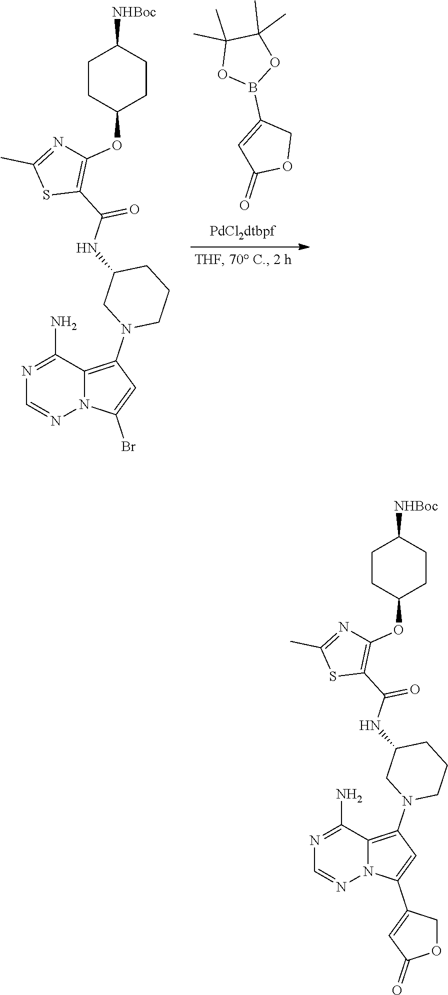 [SMILES: Cc1nc(O[C@H]2CC[C@@H](NC(=O)OC(C)(C)C)CC2)c(C(=O)N[C@@H]2CCCN(c3cc(Br)n4ncnc(N)c34)C2)s1 ; CC1(C)OB(C2=CC(=O)OC2)OC1(C)C ; Cc1nc(O[C@H]2CC[C@@H](NC(=O)OC(C)(C)C)CC2)c(C(=O)N[C@@H]2CCCN(c3cc(C4=CC(=O)OC4)n4ncnc(N)c34)C2)s1]

PdCl₂(dppf)-CH₂Cl₂ adduct (31.4 mg, 0.038 mmol) was added to a stirred solution of Example 977C (250 mg, 0.385 mmol), Example 977D (323 mg, 0.770 mmol) and 2M potassium phosphate, tribasic (0.385 mL, 0.770 mmol) in THF (5 mL) and the reaction mixture was heated at 70° C. for 3 h. Mixture was partitioned between EtOAc and brine solution. The organic layer was dried over Na₂SO₄, filtered, and concentrated. The crude residue was purified by flash chromatography (0-20% MeOH in DCM) to afford Example 977E (70 mg, 22%) as a yellow solid. MS: [M+H]⁺=653.3.

### Example 977F. N—((R)-1-(4-Amino-7-(5-oxo-2,5-dihydrofuran-3-yl)pyrrolo[2,1-f][1,2,4]triazin-5-yl)piperidin-3-yl)-4-(((1s,4S)-4-aminocyclohexyl)oxy)-2-methylthiazole-5-carboxamide,TFA

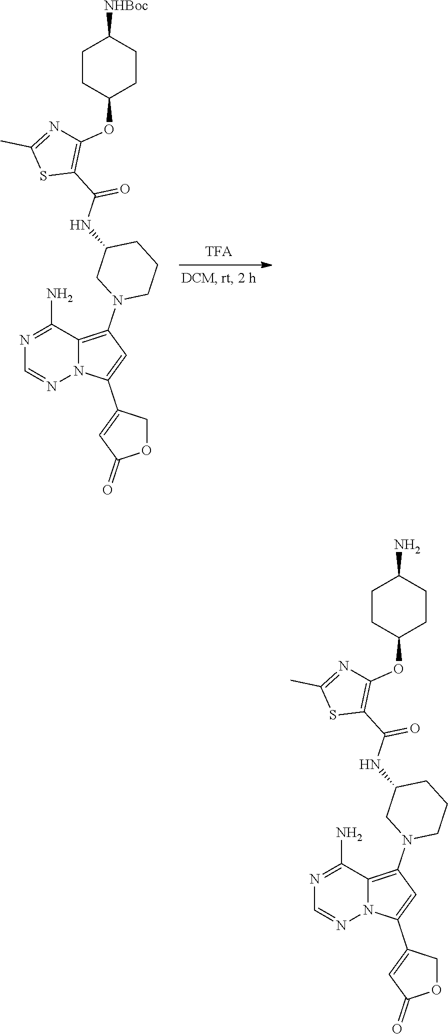 [SMILES: Cc1nc(O[C@H]2CC[C@@H](NC(=O)OC(C)(C)C)CC2)c(C(=O)N[C@@H]2CCCN(c3cc(C4=CC(=O)OC4)n4ncnc(N)c34)C2)s1 ; Cc1nc(O[C@H]2CC[C@@H](N)CC2)c(C(=O)N[C@@H]2CCCN(c3cc(C4=CC(=O)OC4)n4ncnc(N)c34)C2)s1]

To a stirred solution of Example 977E (70 mg, 0.107 mmol) in DCM (2 mL) was added TFA (0.124 mL, 1.61 mmol) and the reaction mixture was stirred at rt for 2 h. Then, mixture was concentrated in vacuo to afford Example 977F (60 mg, 68%) as a yellow solid. [M+H]⁺=553.2.

### Example 977

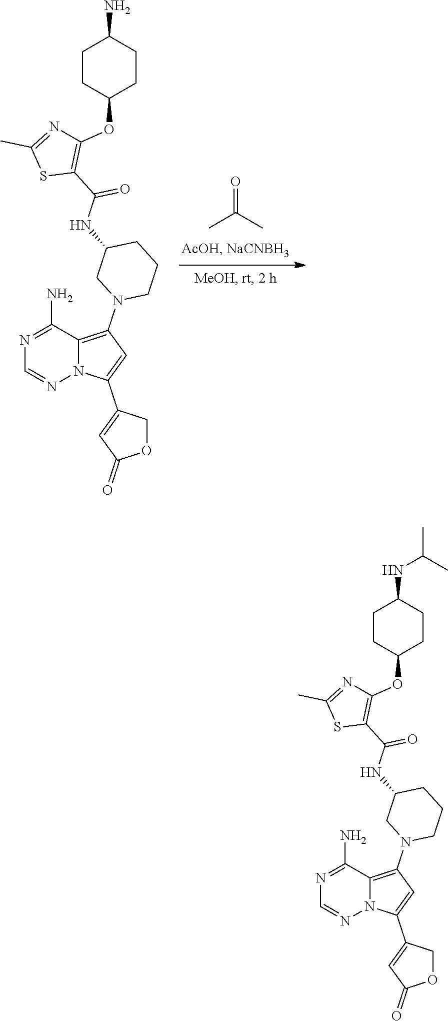

To a solution of Example 977F (30 mg, 0.045 mmol), acetic acid (2.6 μl, 0.045 mmol) and propan-2-one (39.2 mg, 0.675 mmol) in methanol (2 mL) was added sodium cyanoborohydride (5.7 mg, 0.090 mmol) and the reaction mixture was stirred at rt for 2 h. Mixture was diluted with ethyl acetate and filtered. The filtrate was concentrated and purified by preparative HPLC to afford Example 977 (7.5 mg, 27%) as pale yellow solid. LC-MS Method E: RT=1.21 min, [M+H]⁺=593.3; LC-MS Method F: RT=1.04 min, [M+H]⁺=595.3; ¹H NMR (400 MHz, DMSO-d₆) δ ppm 8.47-8.21 (m, 1H), 8.00 (s, 1H), 7.36-7.24 (m, 1H), 7.14 (s, 1H), 6.91-6.74 (m, 1H), 6.64 (s, 1H), 5.39 (s, 2H), 5.11 (br s, 1H), 4.29-4.12 (m, 1H), 3.04-2.90 (m, 1H), 2.68 (s, 4H), 2.60 (s, 3H), 1.99 (br s, 3H), 1.84 (br s, 2H), 1.73-1.47 (m, 6H), 1.24 (s, 3H), 0.97-0.89 (m, 6H).

### Example 978

### Preparation of N—((R)-1-(4-Amino-7-(4-carbamoyl-3-fluorophenyl)pyrrolo[2,1-f][1,2,4]triazin-5-yl)piperidin-3-yl)-4-(((1s,4S)-4-((2-hydroxy-2-methylpropyl)amino)cyclohexyl)oxy)-2-methylthiazole-5-carboxamide

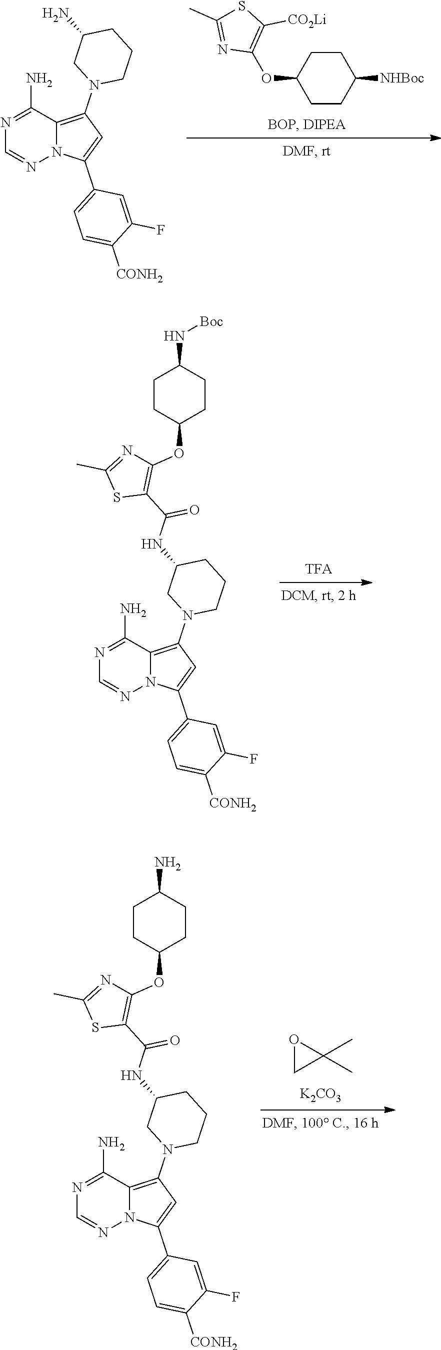

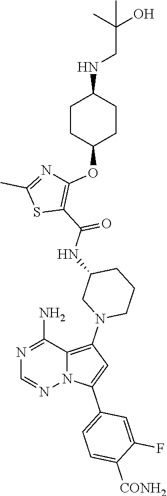 [SMILES: Cc1nc(O[C@H]2CC[C@@H](NCC(C)(C)O)CC2)c(C(=O)N[C@@H]2CCCN(c3cc(-c4ccc(C(N)=O)c(F)c4)n4ncnc(N)c34)C2)s1]

### Example 978A. tert-Butyl ((1S,4s)-4-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)cyclohexyl)carbamate

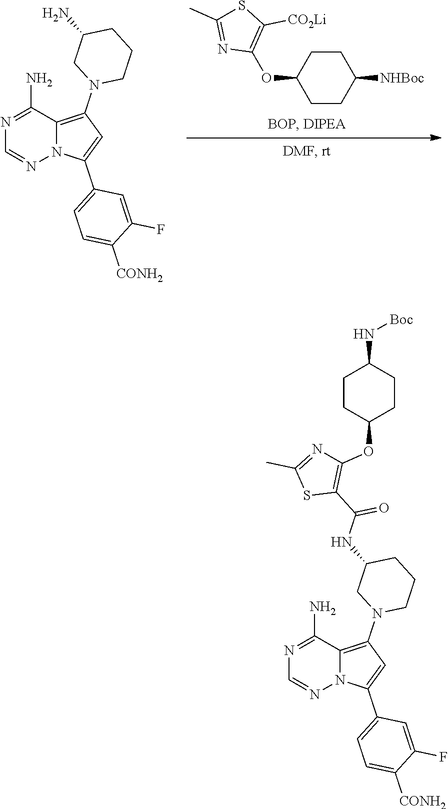

Analogous to the preparation of Example 970G, Example 977B (250 mg, 0.690 mmol), (R)-4-(4-amino-5-(3-aminopiperidin-1-yl)pyrrolo[2,1-f][1,2,4]triazin-7-yl)-2-fluorobenzamide (255 mg, 0.690 mmol), BOP (366 mg, 0.828 mmol) and DIEA (0.361 mL, 2.07 mmol) in DMF (6 mL) afforded Example 978A (370 mg, 64%) as a yellow solid. MS: [M+H]⁺=708.2; ¹H NMR (400 MHz, DMSO-d₆) δ ppm 8.16-8.09 (m, 1H), 8.00 (d, J=8.0 Hz, 1H), 7.92 (s, 1H), 7.74 (s, 1H), 7.68-7.59 (m, 2H), 7.22 (s, 1H), 5.08-4.99 (m, 1H), 4.18-4.08 (m, 1H), 3.11-2.92 (m, 3H), 2.78 (s, 2H), 2.60 (s, 3H), 2.07-1.93 (m, 3H), 1.90-1.79 (m, 2H), 1.78-1.65 (m, 3H), 1.63-1.43 (m, 4H), 1.38 (s, 1H), 1.34 (s, 9H).

### Example 978B. N—((R)-1-(4-Amino-7-(4-carbamoyl-3-fluorophenyl)pyrrolo[2,1-f][1,2,4]triazin-5-yl)piperidin-3-yl)-4-(((1s,4S)-4-aminocyclohexyl)oxy)-2-methylthiazole-5-carboxamide.TFA

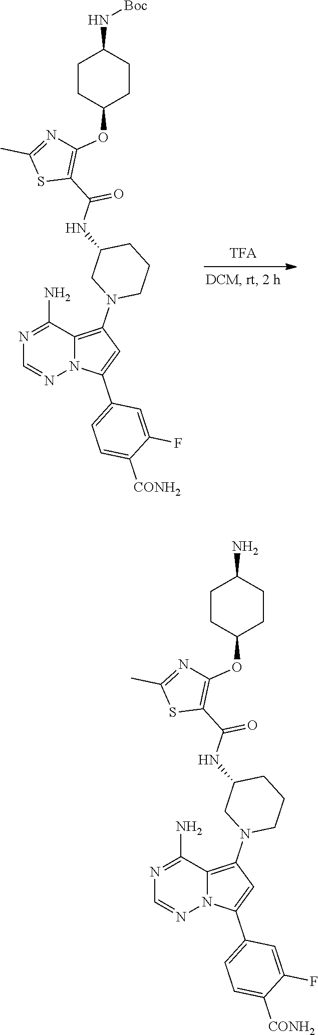 [SMILES: Cc1nc(O[C@H]2CC[C@@H](NC(=O)OC(C)(C)C)CC2)c(C(=O)N[C@@H]2CCCN(c3cc(-c4ccc(C(N)=O)c(F)c4)n4ncnc(N)c34)C2)s1 ; Cc1nc(O[C@H]2CC[C@@H](N)CC2)c(C(=O)N[C@@H]2CCCN(c3cc(-c4ccc(C(N)=O)c(F)c4)n4ncnc(N)c34)C2)s1]

Analogous to the preparation of Example 970H, starting with Example 978A (370 mg, 0.523 mmol) and TFA (0.604 mL, 7.84 mmol) to afford Example 978B (300 mg, 72%) as a yellow solid. MS: [M+H]⁺=608.35; ¹H NMR (400 MHz, DMSO-d₆) δ ppm 8.10 (dd, J=1.5, 13.0 Hz, 1H), 7.99 (d, J=8.5 Hz, 1H), 7.97 (s, 1H), 7.85 (br s, 3H), 7.76 (t, J=8.0 Hz, 2H), 7.66 (br d, J=7.5 Hz, 2H), 7.23 (s, 1H), 7.12 (br s, 1H), 5.01 (br s, 1H), 4.14 (br s, 1H), 3.20-3.10 (m, 2H), 2.91-2.69 (m, 2H), 2.61 (s, 3H), 2.16 (br d, J=14.5 Hz, 2H), 2.00 (br s, 1H), 1.85 (br s, 3H), 1.77 (br d, J=12.0 Hz, 3H), 1.70-1.47 (m, 4H).

### Example 978

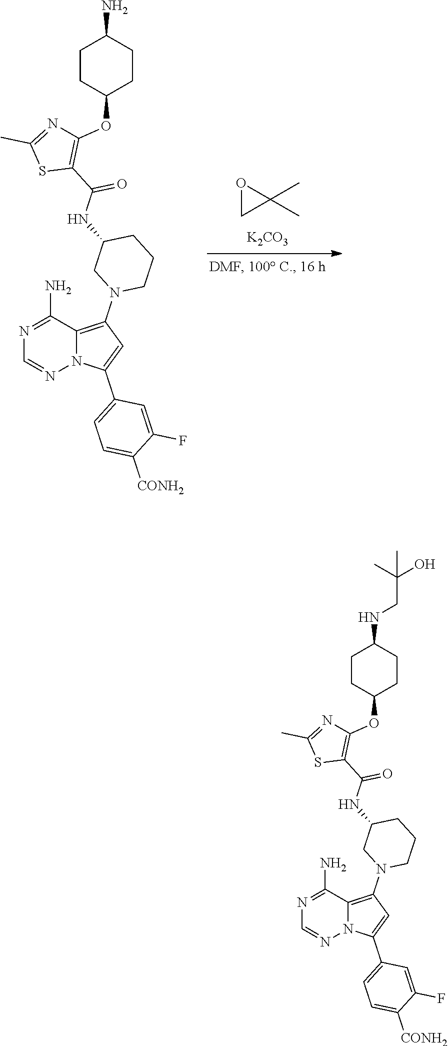

To a stirred solution of Example 978B (50 mg, 0.069 mmol) in DMF (2 mL) was added K₂CO₃ (19.2 mg, 0.139 mmol) and 2,2-dimethyloxirane (15 mg, 0.208 mmol). The reaction mixture was heated at 100° C. for 16 h. It was filtered, concentrated, and purified by preparative HPLC to afford Example 978 (9 mg, 19%) as a pale-yellow solid. LC-MS Method E: RT=1.28 min, [M+H]⁺=680.3; LC-MS Method F: RT=0.96 min, [M+H]⁺=680.3; ¹H NMR (400 MHz, DMSO-d₆) δ ppm 8.12 (dd, J=1.4, 13.1 Hz, 2H), 8.00 (dd, J=1.5, 8.3 Hz, 1H), 7.92 (s, 1H), 7.74 (t, J=8.1 Hz, 1H), 7.64 (br d, J=9.6 Hz, 2H), 7.28 (br s, 1H), 7.21 (s, 1H), 6.93-6.71 (m, 1H), 5.14 (br s, 1H), 4.17 (br s, 2H), 3.01 (br s, 2H), 2.79 (br d, J=17.5 Hz, 2H), 2.61 (s, 3H), 2.42-2.39 (m, 1H), 1.99 (br s, 2H), 1.91 (s, 1H), 1.84 (br s, 3H), 1.66 (br d, J=10.4 Hz, 5H), 1.43 (br s, 3H), 1.19-1.06 (m, 1H), 1.02 (br s, 6H).

### Example 979

### Preparation of N—((R)-1-(4-Amino-7-(4-carbamoyl-3-fluorophenyl)pyrrolo[2,1-f][1,2,4]triazin-5-yl)piperidin-3-yl)-4-(((1s,4S)-4-((2-cyanoethyl)amino)cyclohexyl)oxy)-2-methylthiazole-5-carboxamide

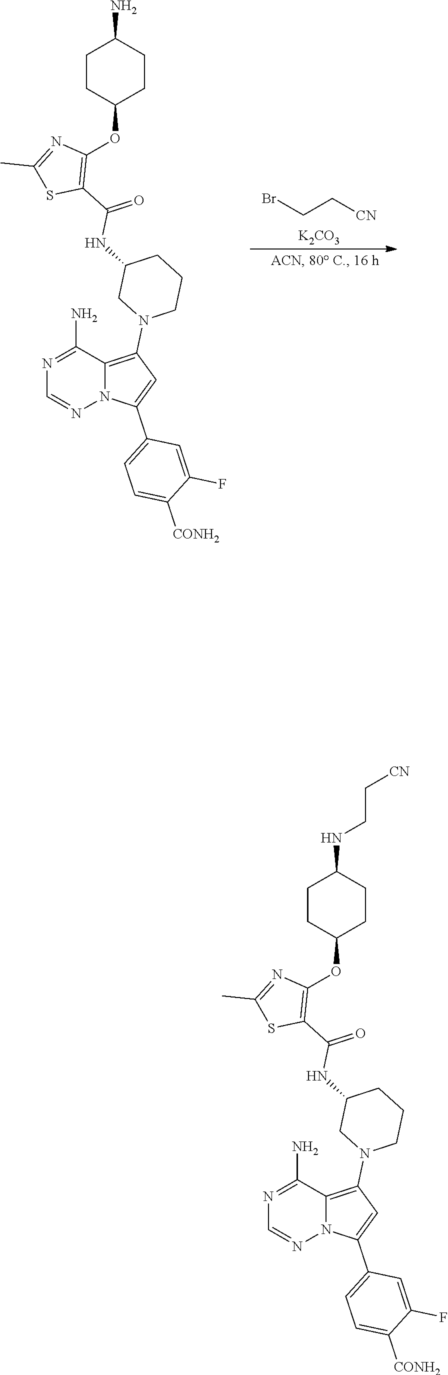

To a stirred solution of Example 978B (50 mg, 0.069 mmol) and K₂CO₃ (9.6 mg, 0.069 mmol) in acetonitrile (2 mL) was added 3-bromopropanenitrile (27.8 mg, 0.208 mmol) and the reaction mixture was heated at 80° C. for 16 h. The mixture was filtered, and filtrate was concentrated and purified by preparative HPLC to afford Example 979 (4.9 mg, 11%) as a pale-yellow solid. LC-MS Method E: RT=1.51 min, [M+H]⁺=661.3; LC-MS Method F: RT=0.94 min, [M+H]⁺=661.3; ¹H NMR (400 MHz, DMSO-d₆) δ ppm 8.14 (br d, J=1.4 Hz, 1H), 8.11 (br d, J=1.4 Hz, 1H), 8.00 (dd, J=1.5, 8.3 Hz, 1H), 7.92 (s, 1H), 7.75 (t, J=8.1 Hz, 1H), 7.64 (br d, J=7.3 Hz, 2H), 7.23 (s, 2H), 6.93-6.74 (m, 1H), 5.12 (br s, 1H), 4.16 (br s, 1H), 2.99 (br s, 2H), 2.77 (br s, 3H), 2.61 (s, 4H), 2.58-2.53 (m, 1H), 1.97 (br s, 3H), 1.86 (br s, 3H), 1.68 (br t, J=11.3 Hz, 6H), 1.41 (br s, 3H).

### Example 980

### Preparation of N—((R)-1-(4-Amino-7-(1-(2,2-difluoroethyl)-6-oxo-1,6-dihydropyridazin-3-yl)pyrrolo[2,1-f][1,2,4]triazin-5-yl)piperidin-3-yl)-2-methyl-4-(((1s,4S)-4-((tetrahydrofuran-3-yl)amino)cyclohexyl)oxy)thiazole-5-carboxamide

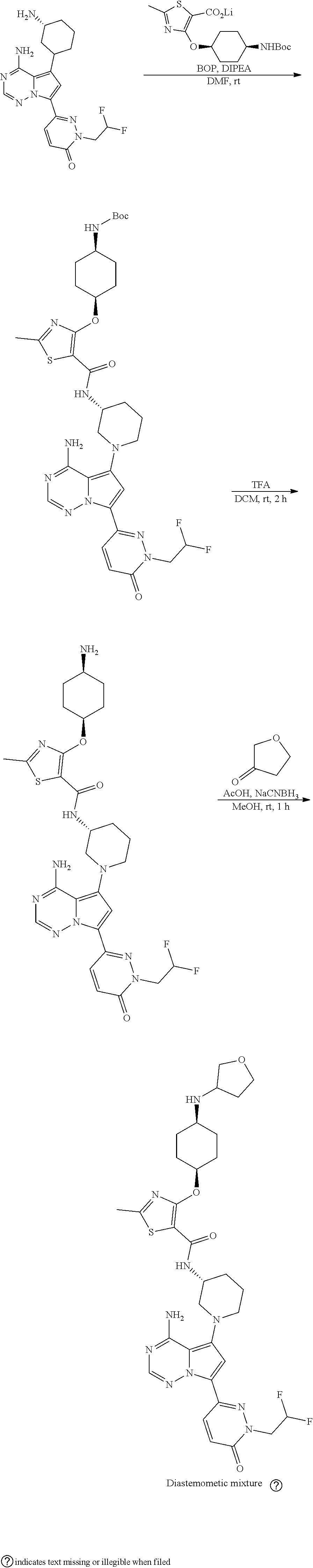

### Example 980A. tert-Butyl ((1S,4s)-4-((5-(((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)carbamoyl)-2-methylthiazol-4-yl)oxy)cyclohexyl)carbamate

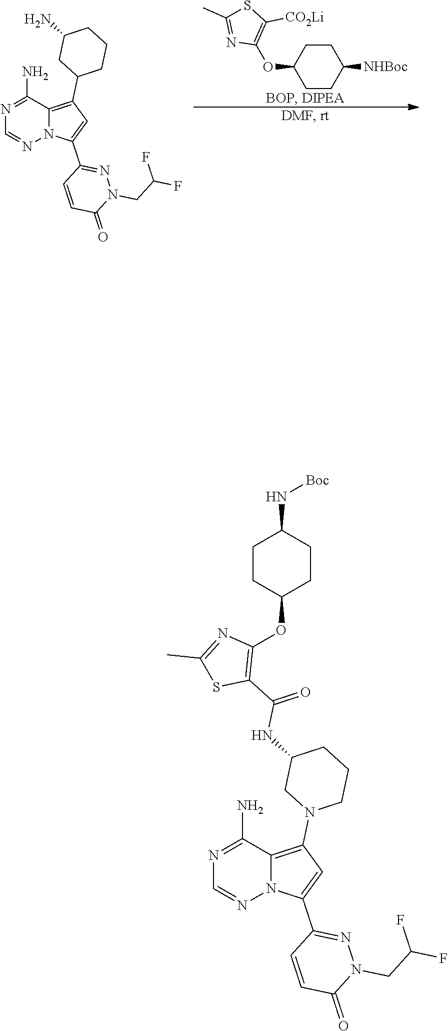

Prepared as described in Example 970, to afford title compound Example 980A (300 mg, 51%) as a yellow solid. MS: [M+H]⁺=729.30; ¹H NMR (400 MHz, DMSO-d₆) δ ppm 8.45 (d, J=10.0 Hz, 1H), 8.16 (br d, J=8.5 Hz, 1H), 7.91-7.85 (m, 1H), 7.69 (br d, J=7.0 Hz, 1H), 7.22-7.15 (m, 1H), 7.01 (s, 1H), 6.77 (br s, 1H), 6.65-6.29 (m, 1H), 5.04 (br s, 1H), 4.56 (dt, J=4.0, 14.3 Hz, 2H), 4.13 (br s, 1H), 2.62-2.58 (m, 3H), 1.99 (br s, 3H), 1.84 (br s, 2H), 1.68 (br d, J=13.5 Hz, 4H), 1.59 (br s, 2H), 1.52 (br d, J=14.5 Hz, 3H), 1.35-1.30 (m, 9H), 1.07-0.95 (m, 4H).

### Example 980B. N—((R)-1-(4-Amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1s,4S)-4-aminocyclohexyl)oxy)-2-methylthiazole-5-carboxamide,TFA

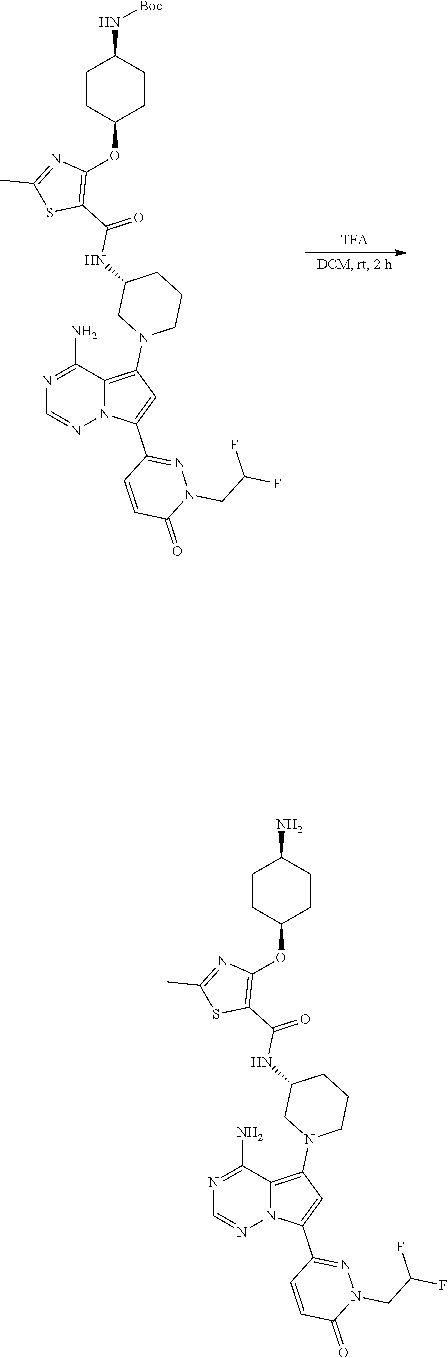 [SMILES: Cc1nc(O[C@H]2CC[C@@H](NC(=O)OC(C)(C)C)CC2)c(C(=O)N[C@@H]2CCCN(c3cc(-c4ccc(=O)n(CC(F)F)n4)n4ncnc(N)c34)C2)s1 ; "TFA" ; Cc1nc(O[C@H]2CC[C@@H](N)CC2)c(C(=O)N[C@@H]2CCCN(c3cc(-c4ccc(=O)n(CC(F)F)n4)n4ncnc(N)c34)C2)s1]

Prepared as described in Example 969G, to afford title compound Example 980B (280 mg, 83%) as a yellow solid. MS: [M+H]⁺=629.30; ¹H NMR (400 MHz, DMSO-d₆) δ ppm 8.47-8.42 (m, 1H), 7.91 (s, 1H), 7.85-7.79 (m, 3H), 7.14 (d, J=10.0 Hz, 1H), 7.00 (s, 2H), 6.63-6.31 (m, 1H), 5.01 (br s, 1H), 4.62-4.53 (m, 2H), 4.16-4.08 (m, 1H), 3.19-3.11 (m, 2H), 3.04-3.00 (m, 1H), 2.62-2.59 (m, 3H), 2.21-2.11 (m, 2H), 2.04-1.94 (m, 2H), 1.88-1.81 (m, 3H), 1.74 (br s, 4H), 1.65-1.53 (m, 4H).

### Example 980

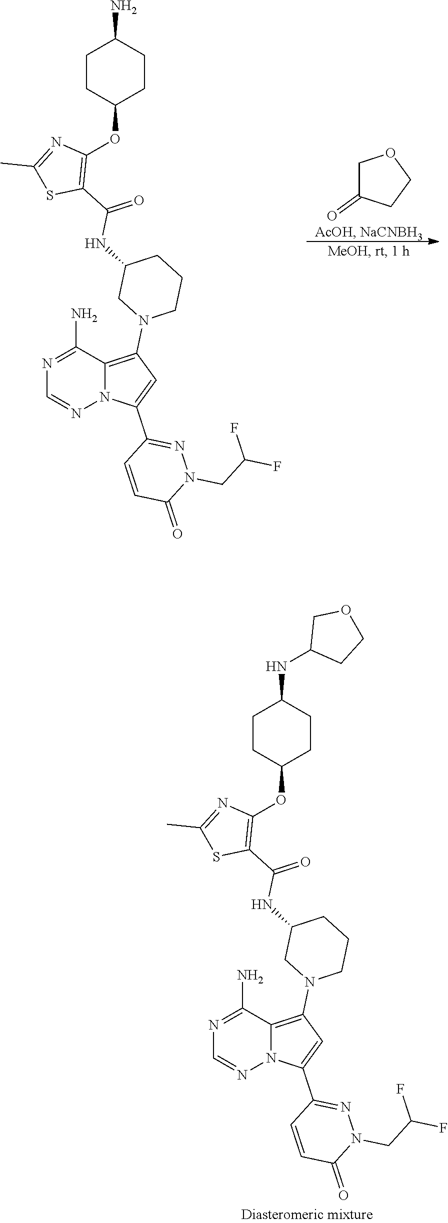 [SMILES: Cc1nc(O[C@H]2CC[C@@H](N)CC2)c(C(=O)N[C@@H]2CCCN(c3cc(-c4ccc(=O)n(CC(F)F)n4)n4ncnc(N)c34)C2)s1 ; O=C1CCOC1 ; Cc1nc(O[C@H]2CC[C@@H](NC3CCOC3)CC2)c(C(=O)N[C@@H]2CCCN(c3cc(-c4ccc(=O)n(CC(F)F)n4)n4ncnc(N)c34)C2)s1]

Analogous to the preparation of Example 969, starting with Example 980B (50 mg, 0.067 mmol), acetic acid (3.9 μl, 0.067 mmol), dihydrofuran-3(2H)-one (29.0 mg, 0.337 mmol) and sodium cyanoborohydride (8.5 mg, 0.135 mmol) in methanol (2 mL) afforded Example 980 (18.1 mg, 38%) as pale-yellow solid. LC-MS Method E: RT=1.53 min, [M+H]⁺=699.3; LC-MS Method F: RT=1.06 min, [M+H]⁺=699.3; ¹H NMR (400 MHz, DMSO-d₆) δ ppm 8.52-8.40 (m, 1H), 8.29-8.08 (m, 1H), 7.89 (s, 1H), 7.34-7.23 (m, 1H), 7.17-7.10 (m, 1H), 7.00 (d, J=1.4 Hz, 1H), 6.85-6.71 (m, 1H), 6.62-6.30 (m, 1H), 5.18-5.02 (m, 1H), 4.62-4.49 (m, 2H), 4.20-4.07 (m, 1H), 3.73-3.48 (m, 3H), 3.23-3.11 (m, 2H), 3.02-2.72 (m, 4H), 2.62-2.58 (m, 3H), 2.06-1.77 (m, 7H), 1.72-1.48 (m, 6H), 1.41-1.16 (m, 3H).

### Example 981

### Preparation of N—((R)-1-(4-Amino-7-(2-(2-hydroxyethoxy)pyrimidin-4-yl)pyrrolo[2,1-f][1,2,4]triazin-5-yl)piperidin-3-yl)-4-(((1s,4S)-4-(dimethylamino)cyclohexyl)oxy)-2-methylthiazole-5-carboxamide

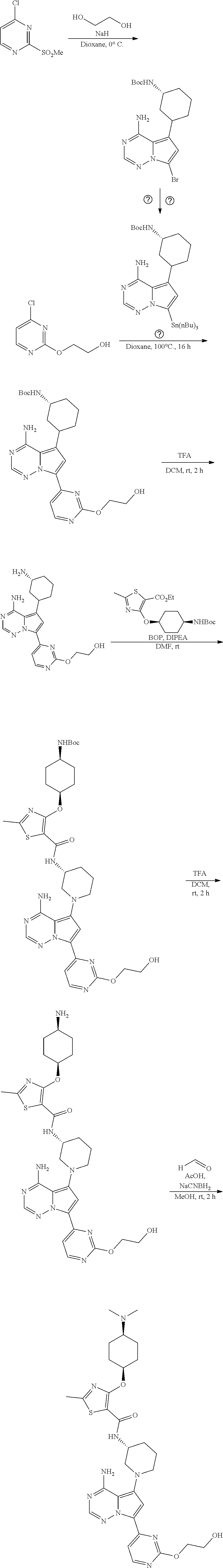

### Example 981A. 2-((4-Chloropyrimidin-2-yl)oxy)ethan-2-ol

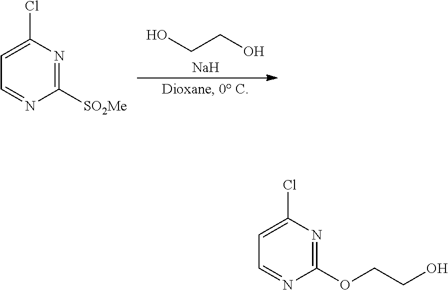

To a stirred solution of ethane-1,2-diol (242 mg, 3.89 mmol) in dioxane (5 mL) was added NaH (156 mg, 3.89 mmol) at 0° C. and the reaction mixture was stirred at same temperature for 15 min followed by the addition of 4-chloro-2-(methylsulfonyl) pyrimidine (500 mg, 2.60 mmol). The reaction mixture was stirred at rt for 16 h, concentrated and partitioned between EtOAc and brine solution. The organic layer was washed with brine solution and dried over sodium sulphate, filtered and concentrated. The residue was purified by flash chromatography (0% to 100% EtOAc/hexanes gradient) to afford Example 981A (210 mg, 43%) as a yellow liquid. MS: [M+H]⁺=175.0; ¹H NMR (400 MHz, DMSO-d₆) δ ppm 8.65-8.55 (m, 1H), 7.35-7.26 (m, 1H), 4.91 (t, J=5.5 Hz, 1H), 4.37-4.30 (m, 2H), 3.72 (d, J=4.5 Hz, 2H).

### Example 981B. tert-Butyl (R)-(1-(4-amino-7-(tributylstannyl)pyrrolo[2,1-f][1,2,4]triazin-5-yl)piperidin-3-yl)carbamate

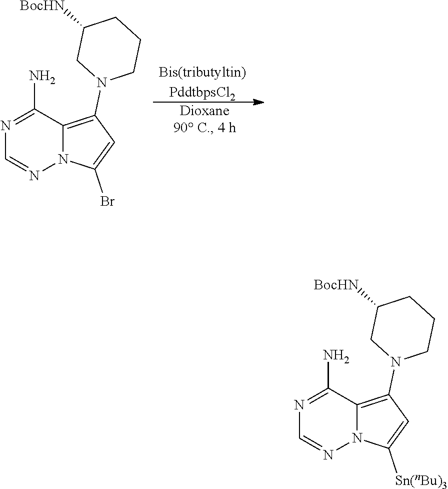

[1,1′-Bis(di-tert-butylphosphino)ferrocene]dichloropalladium(II) (0.317 g, 0.486 mmol) was added to a stirred solution of Intermediate 29 (2.0 g, 4.86 mmol) and bis(tributyltin) (6.14 mL, 12.2 mmol) in dioxane (20 mL). The reaction mixture was heated at 100° C. for 4 h, cooled, diluted with THF, filtered through Celite bed and concentrated in vacuo. The residue was purified by flash chromatography (0% to 60% EtOAc/hexanes gradient) to afford Example 981B (850 mg, 28%) as a yellow gummy solid. MS: [M+H]⁺=625.25; ¹H NMR (400 MHz, DMSO-d₆) δ ppm 7.67 (s, 2H), 7.15-6.91 (m, 1H), 6.80-6.63 (m, 1H), 6.50 (s, 1H), 3.74-3.54 (m, 1H), 3.11-2.97 (m, 1H), 2.90-2.79 (m, 1H), 1.87-1.74 (m, 2H), 1.59-1.47 (m, 6H), 1.39-1.35 (m, 9H), 1.28 (d, J=7.0 Hz, 8H), 1.14-1.06 (m, 6H), 0.83 (t, J=7.3 Hz, 1H).

### Example 981C. tert-Butyl (R)-(1-(4-amino-7-(2-(2-hydroxyethoxy)pyrimidin-4-yl)pyrrolo[2,1-f][1,2,4]triazin-5-yl)piperidin-3-yl)carbamate

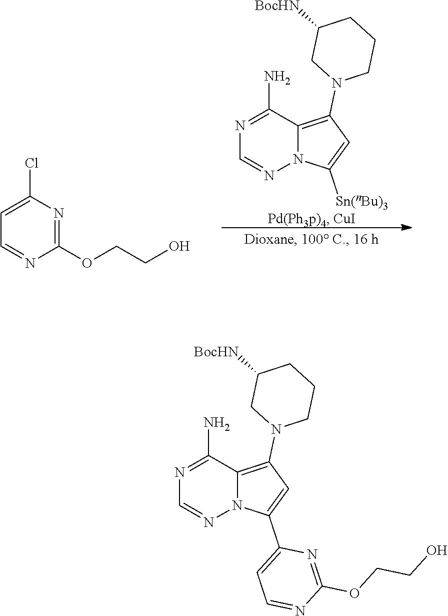

Pd(PPh₃)₄ (93 mg, 0.080 mmol) was added to a solution of Example 981B (500 mg, 0.805 mmol), Example 36A (169 mg, 0.965 mmol) and copper(I) iodide (15.3 mg, 0.080 mmol) in dioxane (10 mL). The reaction mixture was heated at 100° C. for 16 h. After cooling, it was diluted with THF, filtered through Celite bed and concentrated in vacuo. The residue was purified by flash chromatography (0% to 100% EtOAc/hexanes gradient) to afford Example 981C (180 mg, 48%) as a yellow solid. MS: [M+H]⁺=471.25; ¹H NMR (400 MHz, DMSO-d₆) δ ppm 8.63 (d, J=5.0 Hz, 1H), 8.36-8.24 (m, 1H), 8.17 (br d, J=11.5 Hz, 1H), 8.07-7.95 (m, 1H), 7.44-7.33 (m, 1H), 7.18-6.91 (m, 2H), 4.89 (t, J=5.8 Hz, 1H), 4.47-4.30 (m, 2H), 3.81-3.73 (m, 2H), 3.72-3.61 (m, 1H), 3.09 (br d, J=7.5 Hz, 1H), 2.93 (br s, 1H), 1.89-1.58 (m, 3H), 1.39 (s, 12H).

### Example 981D. (R)-2-((4-(4-Amino-5-(3-aminopiperidin-1-yl)pyrrolo[2,1-f][1,2,4]triazin-7-yl)pyrimidin-2-yl)oxy)ethan-1-ol.TFA

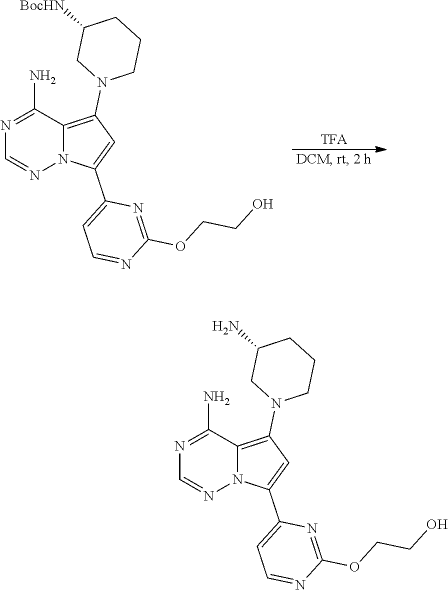 [SMILES: CC(C)(C)OC(=O)N[C@@H]1CCCN(c2cc(-c3ccnc(OCCO)n3)n3ncnc(N)c23)C1 ; Nc1ncnn2c(-c3ccnc(OCCO)n3)cc(N3CCC[C@@H](N)C3)c12]

Analogous to the preparation of Example 970F, starting with Example 981C (180 mg, 0.383 mmol) and TFA (0.44 mL, 5.74 mmol) in DCM (3 mL) afforded Example 981D (150 mg, 56%) as a yellow solid. MS: [M+H]⁺=371.1; ¹H NMR (400 MHz, DMSO-d₆) δ ppm 8.69-8.65 (m, 1H), 8.33-8.28 (m, 1H), 8.08-7.99 (m, 2H), 7.43-7.39 (m, 1H), 7.22-7.13 (m, 1H), 4.83-4.68 (m, 1H), 4.43-4.36 (m, 2H), 3.79-3.74 (m, 2H), 3.29-3.21 (m, 1H), 3.05-2.74 (m, 3H), 2.07-1.56 (m, 5H).

### Example 981E. tert-Butyl ((1S,4s)-4-((5-(((R)-1-(4-amino-7-(2-(2-hydroxyethoxy)pyrimidin-4-yl)pyrrolo[2,1-f][1,2,4]triazin-5-yl)piperidin-3-yl)carbamoyl)-2-methylthiazol-4-yl)oxy)cyclohexyl)carbamate

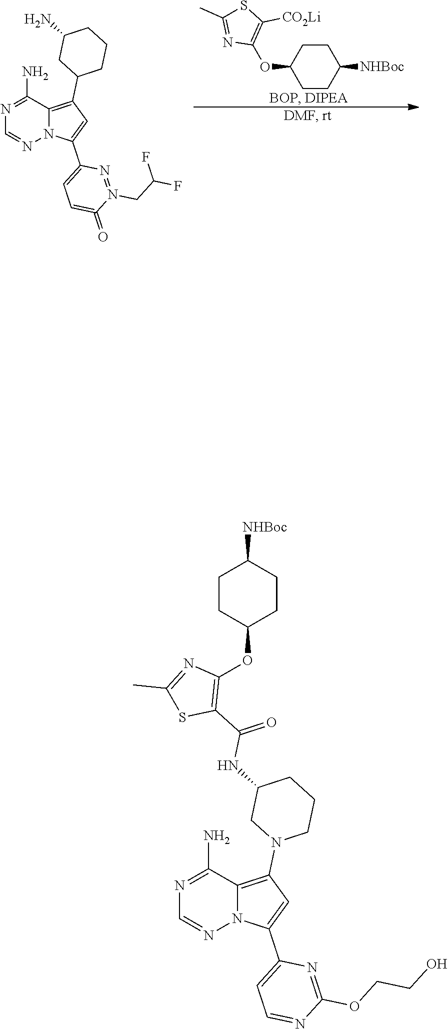

Analogous to the preparation of Example 977C, starting with Example 981D (100 mg, 0.225 mmol) and Example 977B (80 mg, 0.221 mmol), BOP (117 mg, 0.265 mmol) and DIEA (0.116 mL, 0.662 mmol) in DMF (2 mL) afforded Example 981E (150 mg, 53%) as a yellow solid. MS: [M+H]⁺=709.30; ¹H NMR (400 MHz, DMSO-d₆) δ ppm 8.66-8.59 (m, 1H), 8.34-8.22 (m, 2H), 8.02 (s, 1H), 7.40 (s, 1H), 7.26-7.09 (m, 1H), 6.82-6.64 (m, 1H), 5.09-5.00 (m, 1H), 4.91-4.82 (m, 1H), 4.44-4.34 (m, 2H), 4.20-4.06 (m, 1H), 3.82-3.67 (m, 2H), 3.10-2.90 (m, 6H), 2.60 (s, 3H), 2.06-1.98 (m, 3H), 1.88-1.82 (m, 2H), 1.72-1.65 (m, 4H), 1.35-1.27 (m, 12H).

### Example 981F. N—((R)-1-(4-Amino-7-(2-(2-hydroxyethoxy)pyrimidin-4-yl)pyrrolo[2,1-f][1,2,4]triazin-5-yl)piperidin-3-yl)-4-(((1s,4S)-4-aminocyclohexyl)oxy)-2-methylthiazole-5-carboxamide.TFA

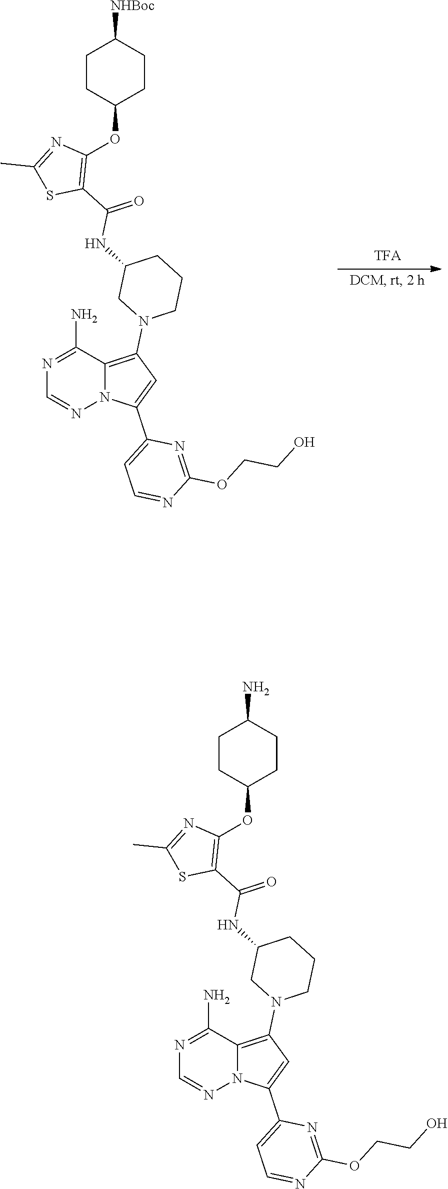 [SMILES: Cc1nc(O[C@H]2CC[C@@H](NC(=O)OC(C)(C)C)CC2)c(C(=O)N[C@@H]2CCCN(c3cc(-c4ccnc(OCCO)n4)n4ncnc(N)c34)C2)s1 ; "TFA" ; Cc1nc(O[C@H]2CC[C@@H](N)CC2)c(C(=O)N[C@@H]2CCCN(c3cc(-c4ccnc(OCCO)n4)n4ncnc(N)c34)C2)s1]

Analogous to the preparation of Example 977F, treatment of Example 981E (150 mg, 0.212 mmol) with TFA afforded Example 981F (120 mg, 55%) as a yellow solid. MS: [M+H]⁺=609.35; ¹H NMR (400 MHz, DMSO-d₆) δ ppm 8.64 (d, J=5.0 Hz, 1H), 8.33-8.29 (m, 1H), 8.04 (s, 1H), 7.86-7.79 (m, 4H), 7.40 (s, 1H), 5.01 (br s, 1H), 4.41-4.35 (m, 2H), 4.17-4.10 (m, 1H), 3.78-3.74 (m, 3H), 3.18-3.12 (m, 2H), 2.62-2.59 (m, 4H), 2.21-2.13 (m, 3H), 1.90-1.81 (m, 4H), 1.79-1.72 (m, 4H), 1.65-1.52 (m, 4H).

### Example 981

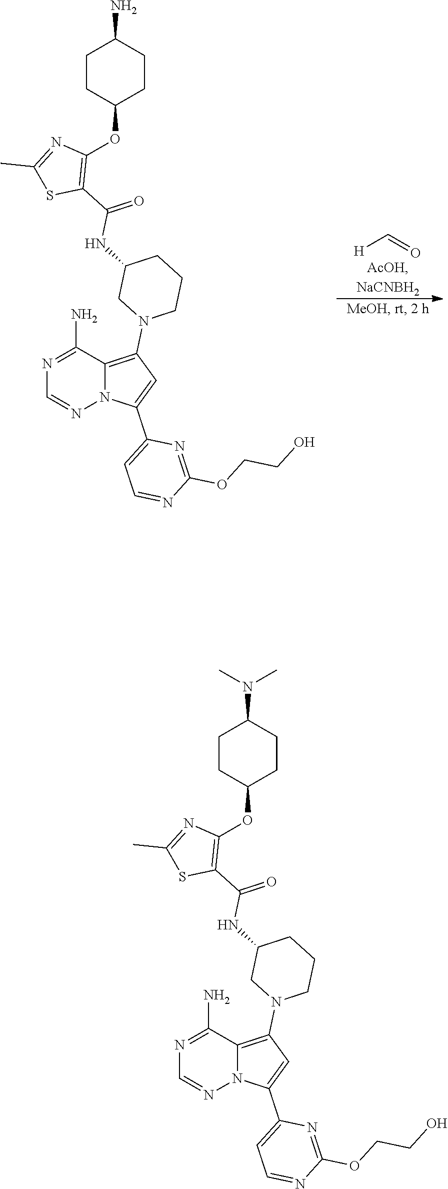 [SMILES: Cc1nc(O[C@H]2CC[C@@H](N)CC2)c(C(=O)N[C@@H]2CCCN(c3cc(-c4ccnc(OCCO)n4)n4ncnc(N)c34)C2)s1 ; C=O ; Cc1nc(O[C@H]2CC[C@@H](N(C)C)CC2)c(C(=O)N[C@@H]2CCCN(c3cc(-c4ccnc(OCCO)n4)n4ncnc(N)c34)C2)s1]

Analogous to the preparation of Example 977, starting with Example 981F (45 mg, 0.062 mmol), acetic acid (7.1 μl, 0.125 mmol), formaldehyde (0.014 mL, 0.187 mmol) and sodium cyanoborohydride (3.9 mg, 0.062 mmol) in methanol (2 mL) afforded Example 981 (8.6 mg, 22%) as pale-yellow solid. LC-MS Method E: RT=1.15 min, [M+H]⁺=637.3; LC-MS Method F: RT=0.95 min, [M+H]⁺=637.3; ¹H NMR (400 MHz, DMSO-d₆) δ ppm 8.63 (d, J=5.3 Hz, 1H), 8.30 (d, J=5.4 Hz, 2H), 8.02 (s, 1H), 7.38 (s, 1H), 7.27-7.14 (m, 1H), 7.04-6.87 (m, 1H), 5.12 (br s, 1H), 4.38 (t, J=5.1 Hz, 2H), 4.19 (br s, 1H), 3.76 (t, J=5.0 Hz, 2H), 2.98 (br s, 2H), 2.60 (s, 3H), 2.15 (br s, 5H), 2.06 (br d, J=16.5 Hz, 4H), 1.84 (br s, 3H), 1.66 (br d, J=10.3 Hz, 5H), 1.48 (br d, J=10.6 Hz, 4H).

### Example 982

### Preparation of N—((R)-1-(4-Amino-7-(4-carbamoyl-3-fluorophenyl)pyrrolo[2,1-f][1,2,4]triazin-5-yl)piperidin-3-yl-4-((5-ethyl-5-azaspiro[3.5]nonan-8-yl)oxy)-2-methylthiazole-5-carboxamide

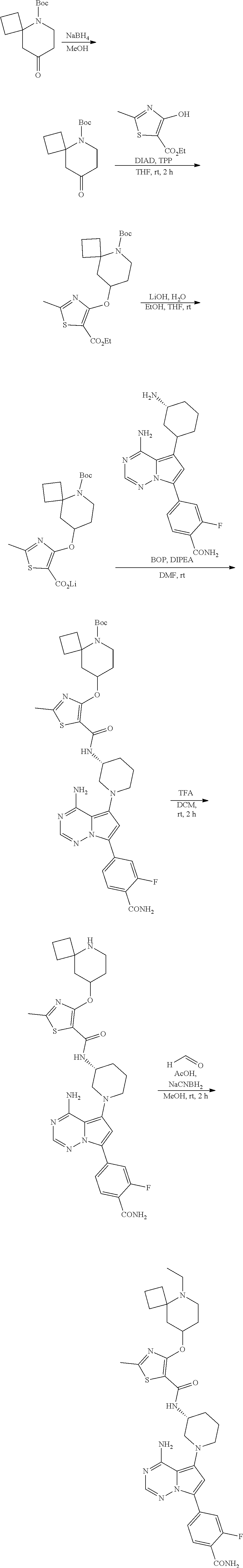

### Example 982A. tert-butyl 8-hydroxy-5-azaspiro[3.5]nonane-5-carboxylate

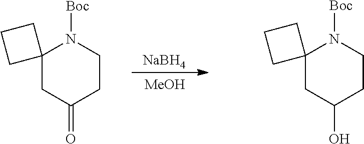 [SMILES: "MeOH" ; CC(C)(C)OC(=O)N1CCC(=O)CC12CCC2 ; "NaBH4" ; CC(C)(C)OC(=O)N1CCC(O)CC12CCC2]

To a stirred solution of tert-butyl 8-oxo-5-azaspiro[3.5]nonane-5-carboxylate (200 mg, 0.836 mmol) in methanol (3 mL) at 0° C. was added NaBH₄ (63.2 mg, 1.67 mmol), stirred for 6 h at rt, concentrated and partitioned between DCM and NH₄Cl solution. The organic layer was washed with brine solution, dried over sodium sulphate, filtered, concentrated to afford Example 982A (195 mg, 97%) as a yellow thick liquid. ¹H NMR (400 MHz, CHCl₃-d) δ ppm 3.98-3.78 (m, 2H), 2.69-2.57 (m, 2H), 2.27-2.20 (m, 1H), 2.17-1.91 (m, 3H), 1.82 (br dd, J=2.3, 12.8 Hz, 1H), 1.75-1.63 (m, 2H), 1.53-1.40 (m, 1H), 1.32-1.22 (m, 1H).

### Example 982B. Ethyl 4-((5-(tert-butoxycarbonyl)-5-azaspiro[3.5]nonan-8-yl)oxy)-2-methylthiazole-5-carboxylate

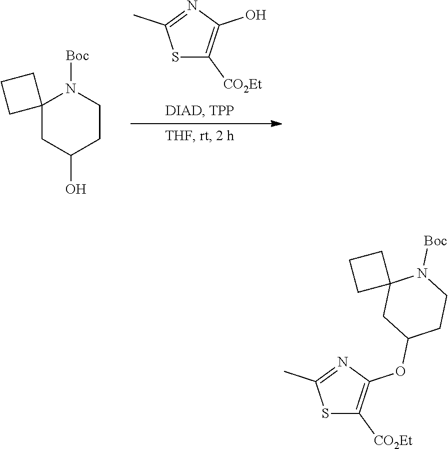 [SMILES: CC(C)(C)OC(=O)N1CCC(O)CC12CCC2 ; CCOC(=O)c1sc(C)nc1O ; CCOC(=O)c1sc(C)nc1OC1CCN(C(=O)OC(C)(C)C)C2(CCC2)C1]

Analogous to the preparation of Example 946B, starting with ethyl 4-hydroxy-2-methylthiazole-5-carboxylate (550 mg, 2.94 mmol), Example 982A (780 mg, 3.23 mmol), triphenylphosphine (1156 mg, 4.41 mmol) and DIAD (0.857 mL, 4.41 mmol) in THF (8 mL) afforded Example 982B (1.2 g, 99%) as racemate which was resolved by SFC (Preparative SFC Conditions: Column/dimensions: Chiralcel OJ-H-2(250×4.6) mm, 5 μm; Cosolvent: 10% of Methanolic ammonia in Methanol; Total Flow: 2 g/min; Back Pressure: 100 bar; Temperature: 40° C.; UV: 254 nm) to get two chiral isomers, mentioned below. First eluting isomer (Iso-1) was taken for next step.

First eluting isomer (Iso-1): 500 mg, 41%, as a yellow gummy solid: SFC Method: RT=2.93 min; 100% cc: [M+H]⁺=411.25; ¹H NMR (400 MHz, CDCl₃) δ ppm 5.23 (tt, J=4.5, 9.0 Hz, 1H), 4.27 (q, J=7.0 Hz, 2H), 3.76 (td, J=4.4, 13.6 Hz, 1H), 2.96 (ddd, J=3.0, 10.4, 13.6 Hz, 1H), 2.61 (s, 3H), 2.58-2.46 (m, 1H), 2.33 (br dd, J=4.8, 12.3 Hz, 2H), 2.19-2.06 (m, 2H), 2.04-1.91 (m, 2H), 1.70 (br d, J=5.5 Hz, 3H), 1.46 (s, 9H), 1.32 (t, J=7.0 Hz, 3H).

Second eluting isomer (Iso-2): 510 mg, 42%, as a yellow gummy solid; SFC Method: RT=3.74 min; 99% ee; [M+H]⁺=411.25; ¹H NMR (400 MHz, CDCl₃) δ ppm 5.30-5.19 (m, 1H), 4.27 (q, J=7.0 Hz, 2H), 3.80-3.71 (m, 1H), 3.01-2.92 (m, 1H), 2.61 (s, 3H), 2.57-2.44 (m, 1H), 2.33 (br dd, J=4.8, 12.3 Hz, 2H), 2.19-1.92 (m, 4H), 1.74-1.51 (m, 3H), 1.46 (s, 9H), 1.31 (t, J=7.3 Hz, 3H).

### Example 982C. Lithium 4-((5-(tert-butoxycarbonyl)-5-azaspiro[3.5]nonan-8-yl)oxy)-2-methylthiazole-5-carboxylate

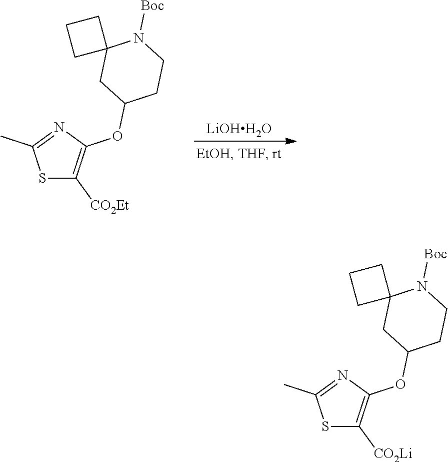

Analogous to the preparation of Example 970B, starting with Example 982B (Iso-1, 500 mg, 1.22 mmol) and LiOH·H₂O (153 mg, 3.65 mmol) afforded Example 982C (450 mg, 91%) as a white solid. MS: [M+H]⁺=383.25; ¹H NMR (400 MHz, DMSO-d₆) δ ppm 5.11-4.98 (m, 1H), 3.72-3.58 (m, 1H), 2.76-2.65 (m, 1H), 2.46 (s, 4H), 2.29-2.22 (m, 1H), 2.18-2.08 (m, 1H), 2.06-1.82 (m, 3H), 1.62 (br s, 3H), 1.40 (s, 9H).

### Example 982D. tert-Butyl 8-((5-(((R)-1-(4-amino-7-(4-carbamoyl-3-fluorophenyl)pyrrolo[2,1-f][1,2,4]triazin-5-yl)piperidin-3-yl)carbamoyl)-2-methylthiazol-4-yl)oxy)-5-azaspiro[3.5]nonane-5-carboxylate

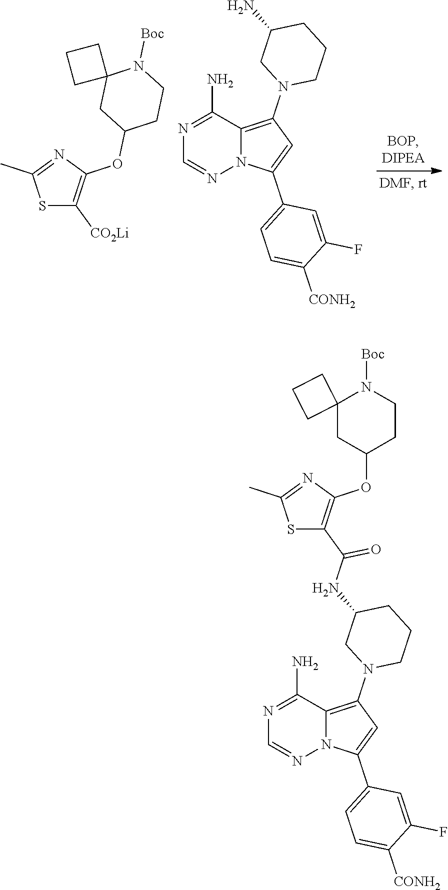

Analogous to the preparation of Example 977C, starting with (R)-4-(4-amino-5-(3-aminopiperidin-1-yl)pyrrolo[2,1-f][1,2,4]triazin-7-yl)-2-fluorobenzamide (157 mg, 0.425 mmol), Example 982C (150 mg, 0.386 mmol), BOP (222 mg, 0.502 mmol) and DIEA (0.202 mL, 1.16 mmol) afforded Example 982D (140 mg, 45%) as a yellow solid. MS: [M+H]⁺=734.40; ¹H NMR (500 MHz, DMSO-d₆) δ ppm 8.18-8.10 (m, 2H), 8.01 (d, J=8.5 Hz, 1H), 7.91 (s, 1H), 7.74 (t, J=7.9 Hz, 1H), 7.69-7.61 (m, 2H), 7.25 (s, 1H), 5.26-5.17 (m, 1H), 4.24-4.12 (m, 1H), 3.74-3.62 (m, 1H), 3.29-3.26 (m, 2H), 2.97-2.94 (m, 1H), 2.64 (s, 4H), 2.44 (br d, J=10.4 Hz, 3H), 2.04 (br s, 3H), 1.93 (br d, J=11.0 Hz, 3H), 1.84 (br s, 3H), 1.62 (br s, 5H), 1.26 (s, 9H).

### Example 982E. 4-((5-Azaspiro[3.5]nonan-8-yl)oxy)-N—((R)-1-(4-amino-7-(4-carbamoyl-3-fluorophenyl)pyrrolo[2,1-f][1,2,4]triazin-5-yl)piperidin-3-yl)-2-methylthiazole-5-carboxamide

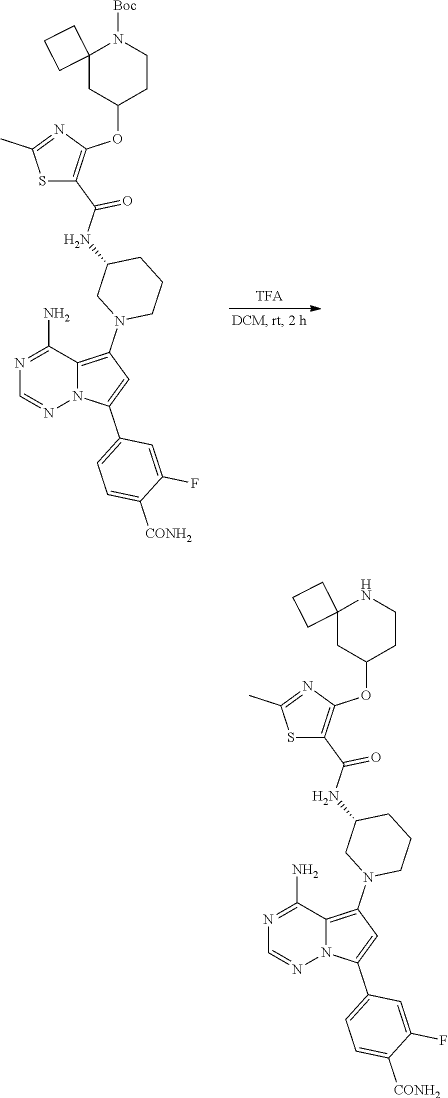 [SMILES: Cc1nc(OC2CCN(C(=O)OC(C)(C)C)C3(CCC3)C2)c(C(=O)N[C@@H]2CCCN(c3cc(-c4ccc(C(N)=O)c(F)c4)n4ncnc(N)c34)C2)s1 ; Cc1nc(OC2CCNC3(CCC3)C2)c(C(=O)N[C@@H]2CCCN(c3cc(-c4ccc(C(N)=O)c(F)c4)n4ncnc(N)c34)C2)s1]

Analogous to the preparation of Example 970H, starting with Example 982D (140 mg, 0.191 mmol) and TFA (0.220 mL, 2.86 mmol) in DCM (2 mL) afforded Example 982E (120 mg, 84%) as a yellow solid. LC-MS Method E: RT=1.27 min, [M+H]⁺=634.3; LC-MS Method F: RT=0.94 min, [M+H]⁺=634.3; 1H NMR (400 MHz, DMSO-d₆) δ ppm 8.13 (dd, J=1.5, 13.0 Hz, 1H), 8.01 (dd, J=1.5, 8.3 Hz, 1H), 7.92 (s, 1H), 7.75 (t, J=8.1 Hz, 1H), 7.65 (br d, J=10.8 Hz, 2H), 7.25 (s, 2H), 5.06-4.96 (m, 1H), 4.18 (br s, 1H), 3.21-3.16 (m, 1H), 3.08-2.70 (m, 4H), 2.62 (s, 3H), 2.38-2.32 (m, 1H), 2.17-2.08 (m, 1H), 2.06-1.95 (m, 4H), 1.90-1.48 (m, 8H).

### Example 982

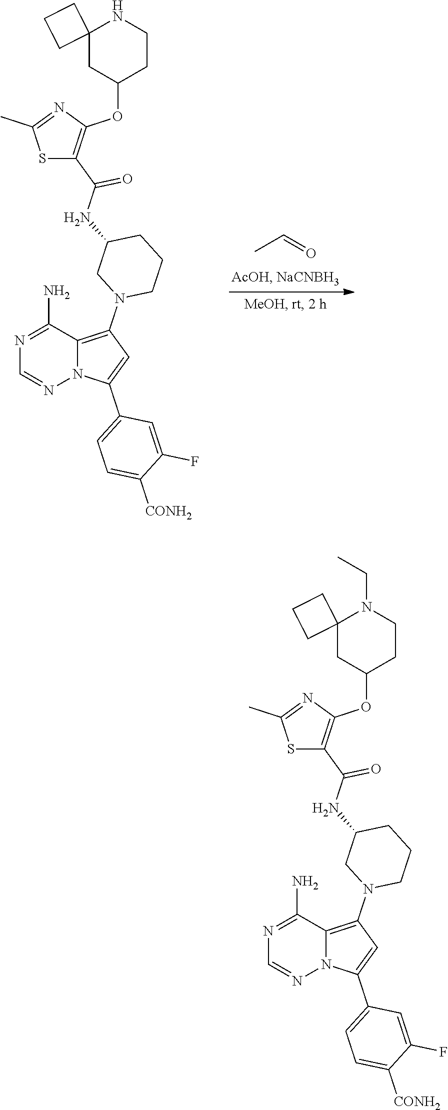

Analogous to the preparation of Example 977, starting with Example 982E (40 mg, 0.053 mmol), acetic acid (3.1 μl, 0.053 mmol), acetaldehyde (23.6 mg, 0.535 mmol) and sodium cyanoborohydride (6.7 mg, 0.107 mmol) in methanol (2 mL) afforded Example 982 (19 mg, 54%) as a pale yellow solid. LC-MS Method E: RT=1.44 min, [M+H]⁺=662.3; LC-MS Method F: RT=0.98 min, [M+H]⁺=662.3; ¹H NMR (400 MHz, DMSO-d₆) δ ppm 8.18-8.10 (m, 1H), 8.01 (dd, J=1.6, 8.3 Hz, 1H), 7.93 (s, 1H), 7.78-7.72 (m, 1H), 7.69-7.62 (m, 2H), 7.24 (s, 1H), 5.01-4.92 (m, 1H), 4.21-4.10 (m, 1H), 3.47-3.39 (m, 1H), 3.29-3.11 (m, 4H), 2.95 (br s, 3H), 2.63 (s, 3H), 2.46-2.36 (m, 3H), 2.30-2.20 (m, 2H), 1.92-1.78 (m, 5H), 1.72-1.56 (m, 4H), 1.33-1.10 (m, 2H), 0.98-0.78 (m, 2H).

### Example 983

### Preparation of N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1r,4R)-4-(dimethylamino)cyclohexyl)methoxy)-2-methylthiazole-5-carboxamide

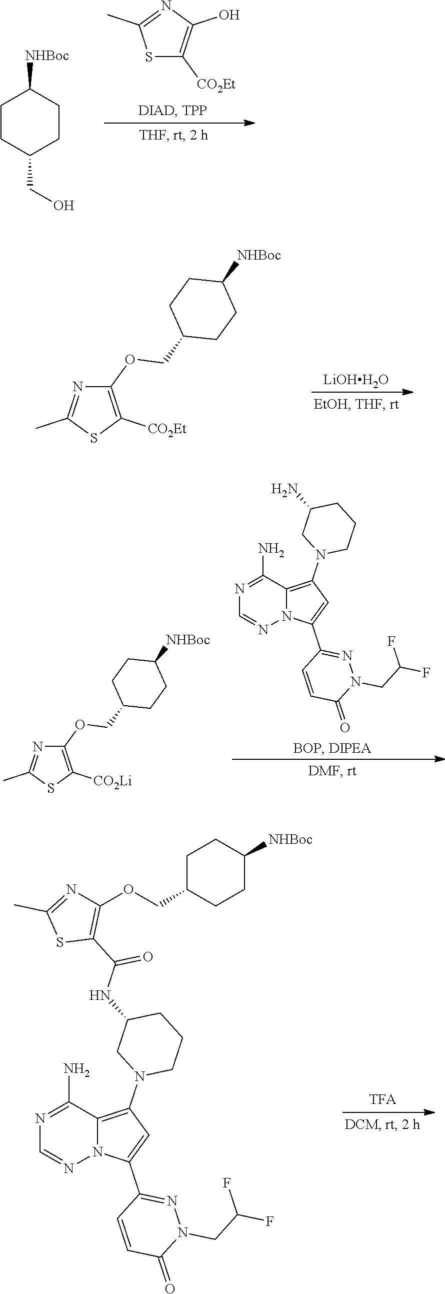

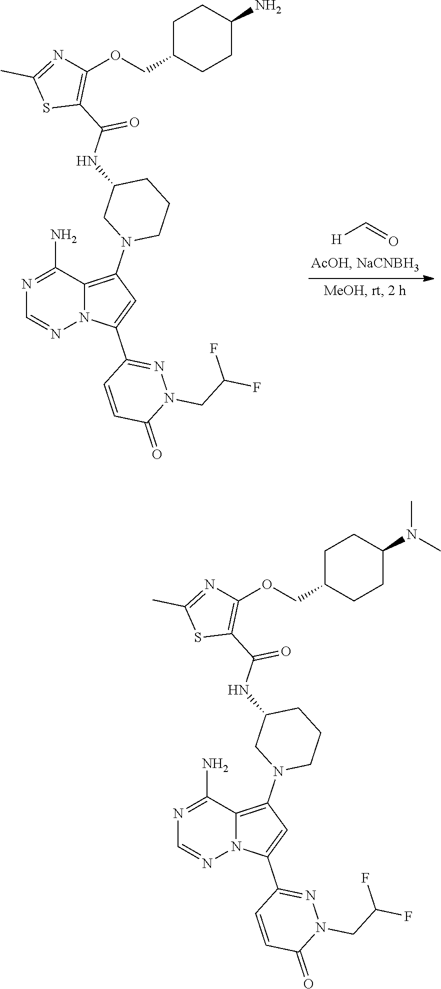

### Example 983A. tert-Butyl 8-hydroxy-5-azaspiro[3.5]nonane-5-carboxylate

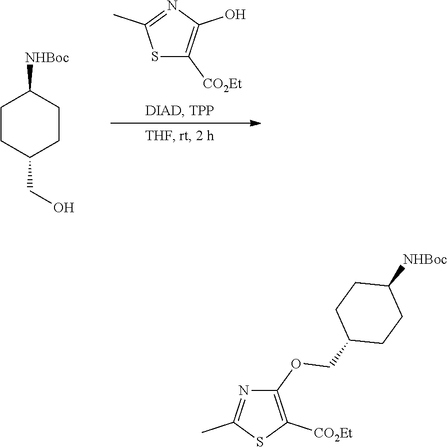 [SMILES: CC(C)(C)OC(=O)N[C@H]1CC[C@H](CO)CC1 ; CCOC(=O)c1sc(C)nc1O ; CCOC(=O)c1sc(C)nc1OC[C@H]1CC[C@H](NC(=O)OC(C)(C)C)CC1]

Analogous to the preparation of Example 970A, starting with ethyl 4-hydroxy-2-methylthiazole-5-carboxylate (2.0 g, 10.7 mmol), tert-butyl ((1r,4r)-4-(hydroxymethyl)cyclohexyl)carbamate (2.94 g, 12.8 mmol), triphenylphosphine (4.20 g, 16.0 mmol) and DIAD (3.12 mL, 16.0 mmol) in THF (100 mL) afforded Example 983A (5.0 g, 86%) as a yellow gummy solid. MS: [M+H]⁺=399.10; ¹H NMR (400 MHz, CDCl₃) δ ppm 4.33-4.20 (m, 4H), 2.66-2.59 (m, 3H), 2.11-2.02 (m, 2H), 1.86-1.74 (m, 1H), 1.52-1.41 (m, 9H), 1.37-1.25 (m, 7H), 1.22-1.07 (m, 4H).

### Example 983B. Lithium 4-(((1r,4r)-4-((tert-butoxycarbonyl)amino)cyclohexyl)methoxy)-2-methylthiazole-5-carboxylate

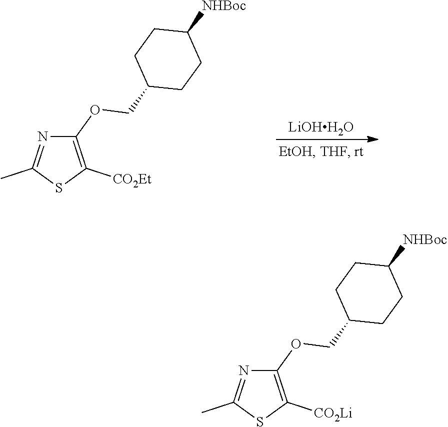

Analogous to the preparation of Example 970B, starting with Example 983A (2.0 g, 5.02 mmol) and lithium hydroxide hydrate (0.632 g, 15.1 mmol) afforded Example 983B (1.6 g, 82%) as a white solid. MS: [M+H]⁺=371.15; ¹H NMR (400 MHz, DMSO-d₆) δ ppm 4.02 (d, J=6.5 Hz, 2H), 3.23-3.08 (m, 1H), 2.45 (s, 3H), 1.80 (br t, J=13.8 Hz, 3H), 1.69-1.56 (m, 1H), 1.38 (s, 9H), 1.17-1.08 (m, 4H), 1.03-0.92 (m, 2H).

### Example 983C. tert-Butyl ((1R,4r)-4-(((5-(((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)carbamoyl)-2-methylthiazol-4-yl)oxy)methyl)cyclohexyl)carbamate

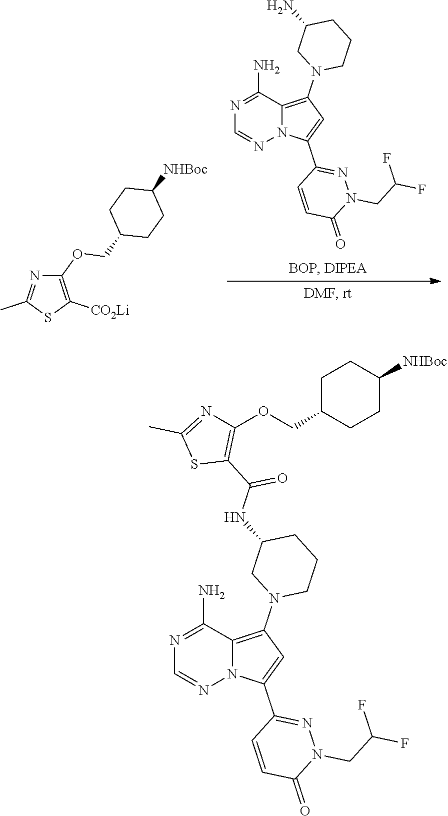

Analogous to the preparation of Example 977C, starting with Example 983B (60 mg, 0.159 mmol), (R)-6-(4-amino-5-(3-aminopiperidin-1-yl)pyrrolo[2,1-f][1,2,4]triazin-7-yl)-2-(2,2-difluoroethyl)pyridazin-3(2H)-one (62.2 mg, 0.159 mmol), BOP (85 mg, 0.191 mmol) and DIEA (0.084 mL, 0.478 mmol) in DMF (2 mL) afforded Example 983C (70 mg, 59%) as a yellow solid. MS: [M+H]⁺=743.4; ¹H NMR (400 MHz, DMSO-d₆) δ ppm 8.53-8.38 (m, 1H), 8.31-8.13 (m, 1H), 7.93-7.83 (m, 1H), 7.43-7.28 (m, 1H), 7.19-7.08 (m, 1H), 7.04-6.97 (m, 1H), 6.64-6.30 (m, 1H), 4.65-4.51 (m, 2H), 4.36-4.09 (m, 4H), 3.21-3.04 (m, 2H), 3.00-2.78 (m, 2H), 2.62-2.60 (m, 3H), 1.90-1.60 (m, 9H), 1.35-1.27 (m, 9H), 1.09-0.97 (m, 6H).

### Example 983D. N—((R)-1-(4-Amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1r,4R)-4-aminocyclohexyl)methoxy)-2-methylthiazole-5-carboxamide

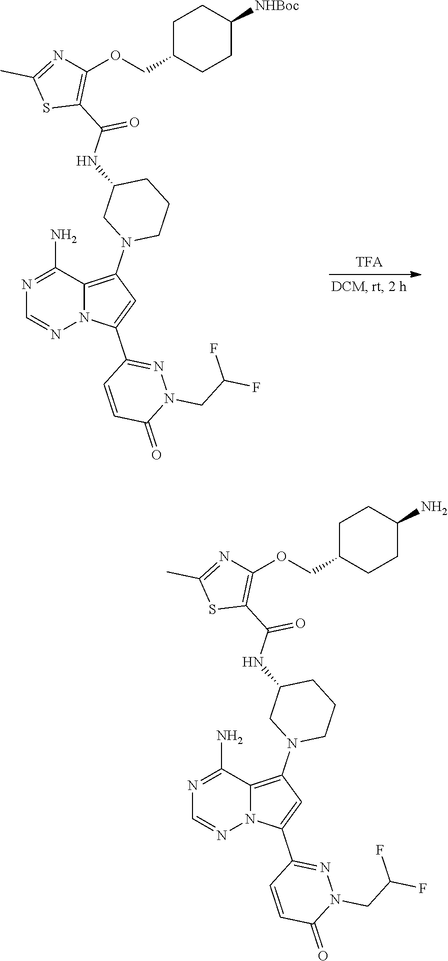 [SMILES: Cc1nc(OC[C@H]2CC[C@H](NC(=O)OC(C)(C)C)CC2)c(C(=O)N[C@@H]2CCCN(c3cc(-c4ccc(=O)n(CC(F)F)n4)n4ncnc(N)c34)C2)s1 ; Cc1nc(OC[C@H]2CC[C@H](N)CC2)c(C(=O)N[C@@H]2CCCN(c3cc(-c4ccc(=O)n(CC(F)F)n4)n4ncnc(N)c34)C2)s1]

Analogous to the preparation of Example 977F, starting with Example 983C (75 mg, 0.101 mmol) and TFA (0.156 mL, 2.019 mmol) in DCM (2 mL) afforded Example 983D (60 mg, 83%) as a yellow solid. MS: [M+H]⁺=643.30.

### Example 983

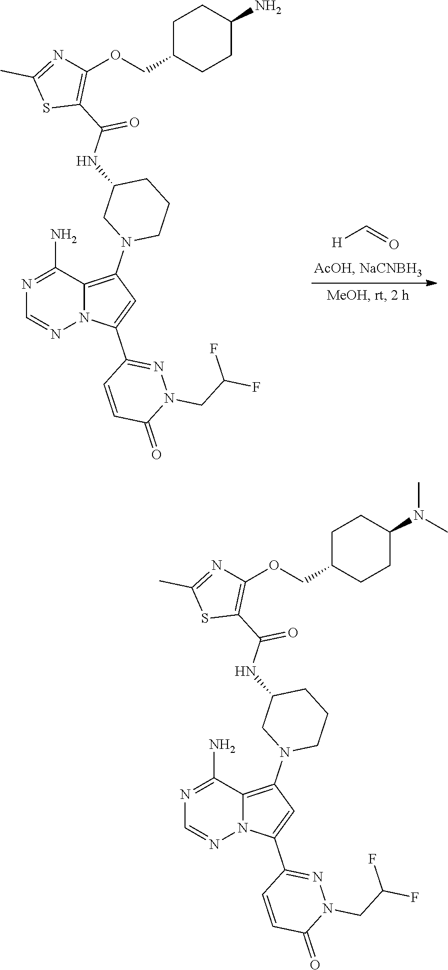

Analogous to the preparation of Example 977, starting with Example 983D (30 mg, 0.040 mmol), formaldehyde (37% aqueous solution, 8.9 μl, 0.119 mmol), acetic acid (2.3 μl, 0.040 mmol) and sodium cyanoborohydride (5.0 mg, 0.079 mmol) afforded Example 983 (6.7 mg, 25%) as a pale-yellow solid. LC-MS Method E: RT=1.28 min, [M+H]⁺=671.3; LC-MS Method F: RT=1.10 min, [M+H]⁺=671.3; ¹H NMR (400 MHz, DMSO-d₆) δ ppm 8.47 (d, J=9.9 Hz, 1H), 8.21 (br s, 1H), 7.90 (s, 1H), 7.43 (br s, 1H), 7.13 (d, J=9.9 Hz, 1H), 7.01 (s, 1H), 6.85-6.69 (m, 1H), 6.63-6.30 (m, 1H), 4.65-4.52 (m, 2H), 4.34-4.13 (m, 3H), 3.29-3.15 (m, 1H), 2.90 (br s, 2H), 2.60 (s, 3H), 2.07 (br s, 6H), 1.96-1.83 (m, 1H), 1.78 (br d, J=12.3 Hz, 5H), 1.65 (br s, 3H), 1.10-0.69 (m, 4H).

### Example 984

### N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-4-((1r,3R)-3-((dimethylamino)methyl)cyclobutoxy)-2-methylthiazole-5-carboxamide

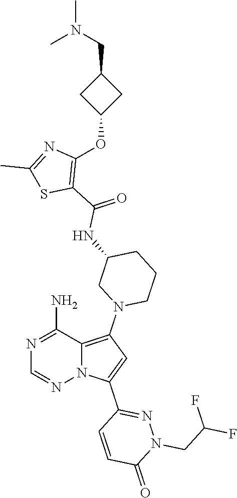 [SMILES: Cc1nc(O[C@H]2C[C@H](CN(C)C)C2)c(C(=O)N[C@@H]2CCCN(c3cc(-c4ccc(=O)n(CC(F)F)n4)n4ncnc(N)c34)C2)s1]

Example 984 was prepared using the same procedure as shown in Example 983. LC-MS Method E: RT=1.27 min, [M+H]⁺=643.3; LC-MS Method F: RT=1.04 min, [M+H]⁺=643.3; ¹H NMR (400 MHz, DMSO-d₆) δ=8.44 (d, J=10.0 Hz, 1H), 8.22 (br s, 1H), 7.88 (s, 1H), 7.42 (br s, 1H), 7.13 (d, J=10.0 Hz, 1H), 7.03 (s, 1H), 6.83 (br s, 1H), 6.48 (tt, J=4.0, 55.8 Hz, 1H), 5.27 (quin, J=6.3 Hz, 1H), 4.57 (dt, J=4.3, 14.4 Hz, 2H), 4.18 (br s, 1H), 3.15 (br s, 1H), 3.03-2.69 (m, 4H), 2.59 (s, 4H), 2.43 (br s, 5H), 2.35-2.15 (m, 4H), 1.84 (br s, 3H), 1.63 (br s, 1H); ¹⁹F NMR (400 MHz, CDCl₃) δ ppm −122.21.

### Example 985

### Preparation of N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1r,4R)-4-(dimethylamino)cyclohexyl)methoxy)-2-methylthiazole-5-carboxamide

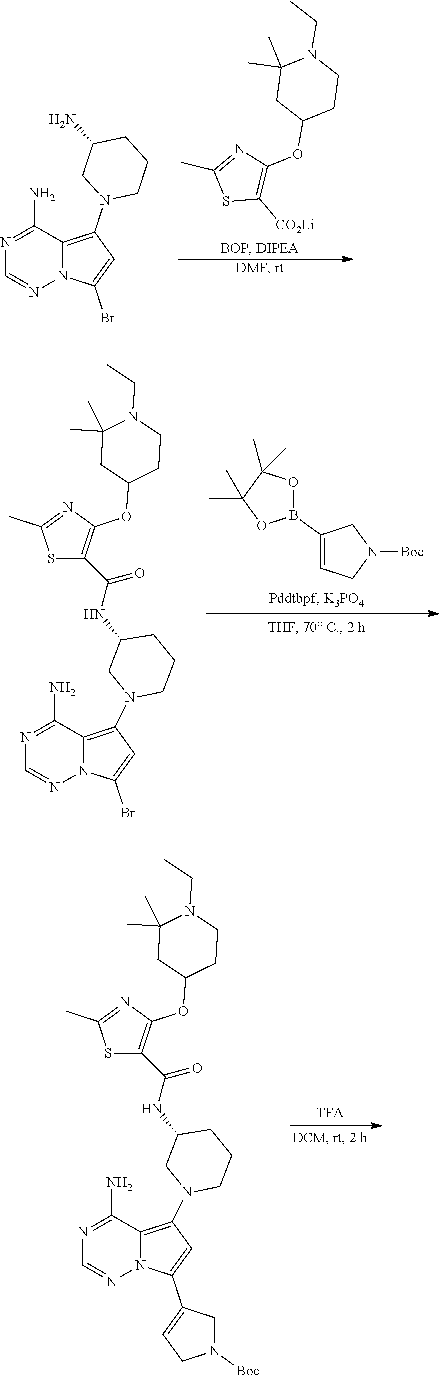

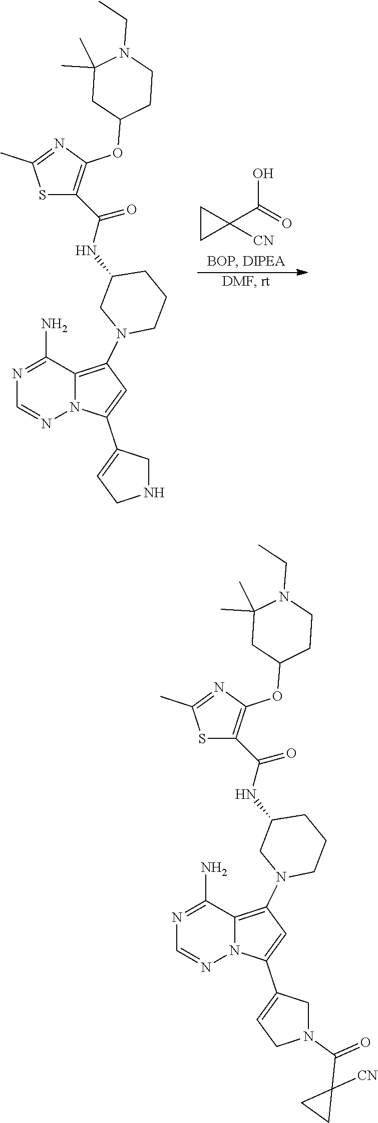

### Example 985A. N—((R)-1-(4-Amino-7-bromopyrrolo[2,1-f][1,2,4]triazin-5-yl)piperidin-3-yl)-4-((1-ethyl-2,2-dimethylpiperidin-4-yl)oxy)-2-methylthiazole-5-carboxamide

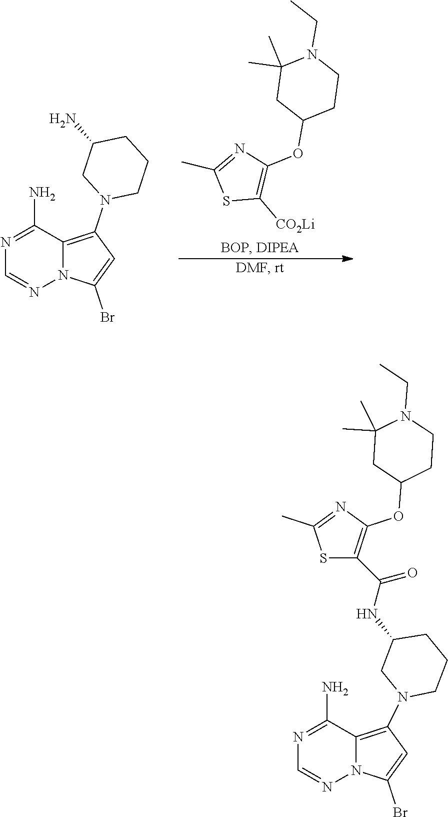

Analogous to the preparation of Example 977C, starting with Intermediate 61, TFA salt (1076 mg, 2.53 mmol), Example 946E (700 mg, 2.30 mmol), BOP (1.22 g, 2.76 mmol) and DIEA (1.21 mL, 6.90 mmol) afforded Example 985A (560 mg, 40%) as a yellow solid. MS: [M+H]⁺=593.10; ¹H NMR (400 MHz, DMSO-d₆) δ ppm 8.83-8.59 (m, 1H), 8.09 (br dd, J=2.0, 7.0 Hz, 1H), 7.86-7.72 (m, 1H), 7.35-7.14 (m, 1H), 6.86-6.71 (m, 1H), 5.80-5.65 (m, 1H), 5.30-5.06 (m, 1H), 4.20-4.06 (m, 1H), 3.70-3.60 (m, 1H), 3.20-3.03 (m, 2H), 2.96-2.71 (m, 4H), 2.64-2.57 (m, 3H), 2.40-2.20 (m, 2H), 1.91-1.73 (m, 4H), 1.65-1.51 (m, 1H), 1.42-1.16 (m, 8H), 1.08-0.86 (m, 2H).

### Example 985B. tert-Butyl 3-(4-amino-5-((3R)-3-(4-((1-ethyl-2,2-dimethylpiperidin-4-yl)oxy)-2-methylthiazole-5-carboxamido)piperidin-1-yl)pyrrolo[2,1-f][1,2,4]triazin-7-yl)-2,5-dihydro-1H-pyrrole-1-carboxylate

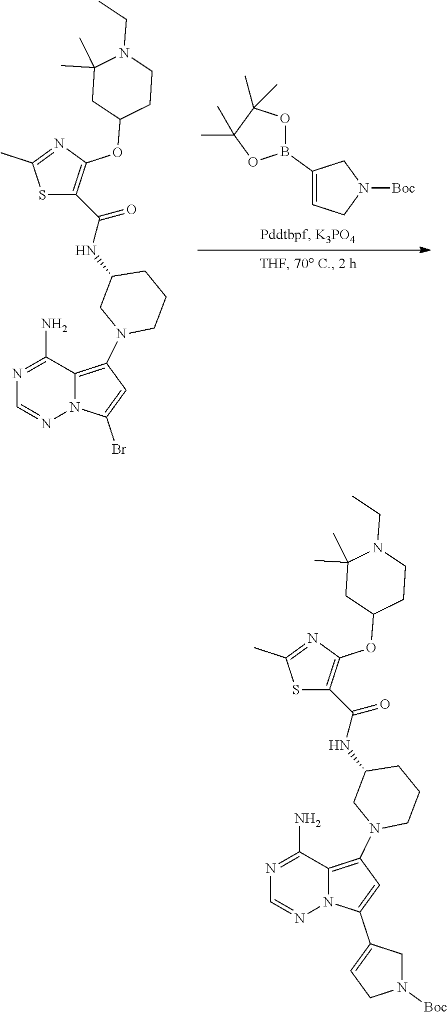 [SMILES: CCN1CCC(Oc2nc(C)sc2C(=O)N[C@@H]2CCCN(c3cc(Br)n4ncnc(N)c34)C2)CC1(C)C ; CC(C)(C)OC(=O)N1CC=C(B2OC(C)(C)C(C)(C)O2)C1 ; CCN1CCC(Oc2nc(C)sc2C(=O)N[C@@H]2CCCN(c3cc(C4=CCN(C(=O)OC(C)(C)C)C4)n4ncnc(N)c34)C2)CC1(C)C]

[1,1′-Bis(di-tert-butylphosphino)ferrocene]dichloropalladium(II) (37.0 mg, 0.057 mmol) was added to a stirred solution of Example 985A (560 mg, 0.947 mmol), tert-butyl 3-(4,4,5,5-tetramethyl-1,3,2-dioxaborolan-2-yl)-2,5-dihydro-1H-pyrrole-1-carboxylate (419 mg, 1.42 mmol) and potassium phosphate, tribasic (2M, 0.947 mL, 1.89 mmol) in THF (5 mL). Reaction mixture was concentrated under reduced pressure to afford crude product which was purified by preparative HPLC to afford Example 985B (500 mg, 74%) as a yellow solid. MS: [M+H]⁺=680.40; ¹H NMR (400 MHz, DMSO-d₆) δ ppm 8.78-8.67 (m, 1H), 8.13-7.91 (m, 1H), 7.90-7.79 (m, 1H), 7.42-7.16 (m, 1H), 6.82-6.61 (m, 2H), 5.78-5.71 (m, 2H), 5.10-4.95 (m, 1H), 4.48-4.40 (m, 2H), 4.28-4.02 (m, 3H), 3.21-3.01 (m, 1H), 2.96-2.73 (m, 3H), 2.64-2.56 (m, 3H), 2.37-2.19 (m, 1H), 2.14-1.96 (m, 1H), 1.92-1.71 (m, 4H), 1.66-1.11 (m, 17H), 1.05-0.87 (m, 5H).

### Example 985C. N—((R)-1-(4-Amino-7-(2,5-dihydro-1H-pyrrol-3-yl)pyrrolo[2,1-f][1,2,4]triazin-5-yl)piperidin-3-yl)-4-((1-ethyl-2,2-dimethylpiperidin-4-yl)oxy)-2-methylthiazole-5-carboxamide.TFA

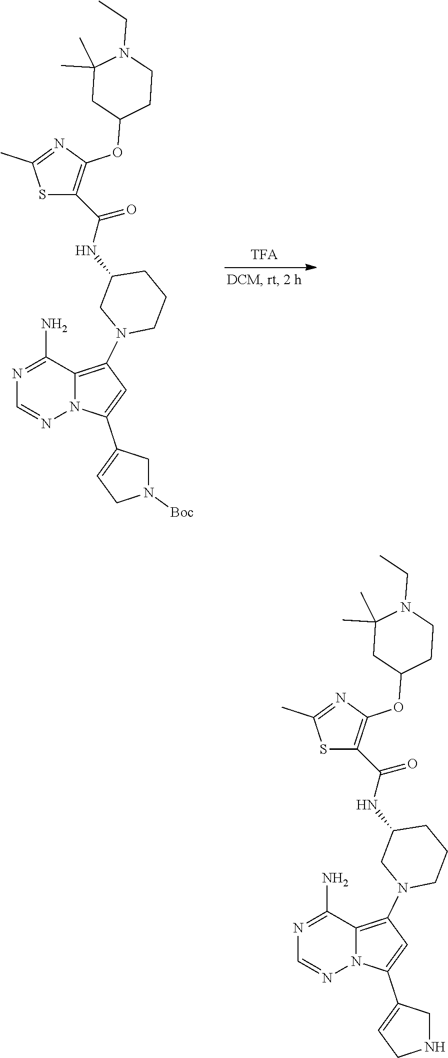 [SMILES: CCN1CCC(Oc2nc(C)sc2C(=O)N[C@@H]2CCCN(c3cc(C4=CCN(C(=O)OC(C)(C)C)C4)n4ncnc(N)c34)C2)CC1(C)C ; CCN1CCC(Oc2nc(C)sc2C(=O)N[C@@H]2CCCN(c3cc(C4=CCNC4)n4ncnc(N)c34)C2)CC1(C)C]

Analogous to the preparation of Example 977F, starting with Example 985B (500 mg, 0.735 mmol) and TFA (0.85 mL, 11.0 mmol) in DCM (10 mL) afforded Example 985C (350 mg, 66%) as a yellow solid. MS: [M+H]⁺=580.50; ¹H NMR (400 MHz, DMSO-d₆) δ ppm 9.35 (br s, 2H), 9.10 (br s, 1H), 8.36-8.19 (m, 1H), 7.92 (s, 1H), 7.21 (br s, 1H), 6.85-6.80 (m, 1H), 6.73 (d, J=2.0 Hz, 1H), 5.25-5.17 (m, 1H), 4.40 (br s, 2H), 4.19 (br s, 3H), 3.69-3.61 (m, 2H), 3.24-3.10 (m, 3H), 2.93 (br s, 2H), 2.64-2.59 (m, 3H), 2.42-2.16 (m, 3H), 1.96-1.74 (m, 5H), 1.41-1.31 (m, 6H), 1.29-1.22 (m, 3H).

### Example 985

### N—((R)-1-(4-Amino-7-(1-(1-cyanocyclopropane-1-carbonyl)-2,5-dihydro-1H-pyrrol-3-yl)pyrrolo[2,1-f][1,2,4]triazin-5-yl)piperidin-3-yl)-4-((1-ethyl-2,2-dimethylpiperidin-4-yl)oxy)-2-methylthiazole-5-carboxamide

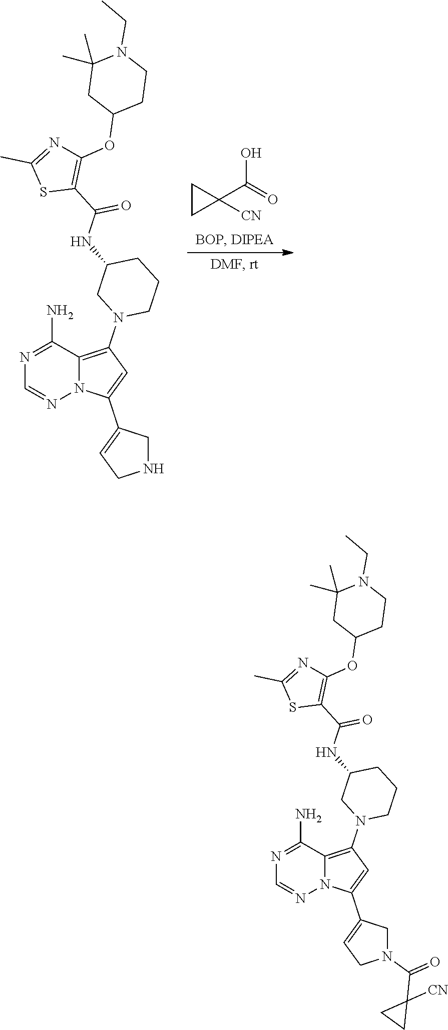 [SMILES: CCN1CCC(Oc2nc(C)sc2C(=O)N[C@@H]2CCCN(c3cc(C4=CCNC4)n4ncnc(N)c34)C2)CC1(C)C ; N#CC1(C(=O)O)CC1 ; CCN1CCC(Oc2nc(C)sc2C(=O)N[C@@H]2CCCN(c3cc(C4=CCN(C(=O)C5(C#N)CC5)C4)n4ncnc(N)c34)C2)CC1(C)C]

Reaction of Example 985C (40 mg, 0.058 mmol), 1-cyanocyclopropane-1-carboxylic acid (6.4 mg, 0.058 mmol), BOP (38.2 mg, 0.086 mmol) and DIEA (0.030 mL, 0.173 mmol) in DMF (1 mL) afforded after HPLC purification Example 985 (27.8 mg, 72%) as a pale-yellow solid. LC-MS Method E: RT=1.52 min, [M+H]⁺=673.3; LC-MS Method F: RT=1.07 min, [M+H]⁺=673.3; ¹H NMR (400 MHz, DMSO-d₆) δ ppm 8.84-8.75 (m, 1H), 8.18-8.01 (m, 1H), 7.90 (s, 1H), 6.86-6.71 (m, 3H), 5.19 (br s, 1H), 5.00 (br s, 1H), 4.80 (br s, 1H), 4.61 (br s, 1H), 4.39 (br s, 1H), 4.21-4.08 (m, 1H), 3.70-3.61 (m, 1H), 2.99-2.91 (m, 2H), 2.67-2.58 (m, 4H), 2.57-2.53 (m, 2H), 1.82 (br d, J=11.1 Hz, 5H), 1.69-1.45 (m, 6H), 1.40-1.30 (m, 7H), 1.29-1.16 (m, 5H).

### Example 986

### Preparation of N—((R)-1-(4-amino-7-(1-(2,2-difluoroethyl)-6-oxo-1,6-dihydropyridazin-3-yl)pyrrolo[2,1-f][1,2,4]triazin-5-yl)piperidin-3-yl)-4-(((1r,4R)-4-(dimethylamino)cyclohexyl)methoxy)-2-methylthiazole-5-carboxamide

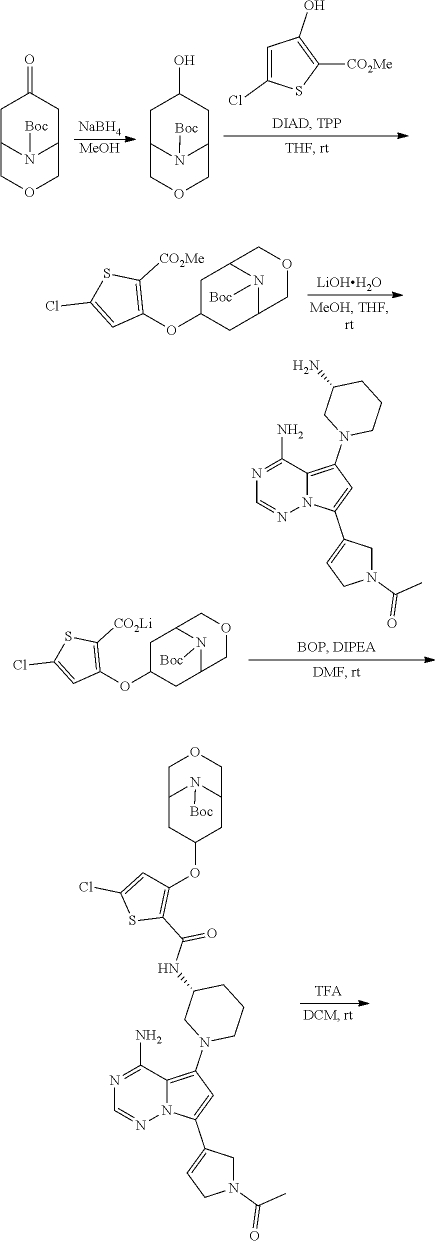

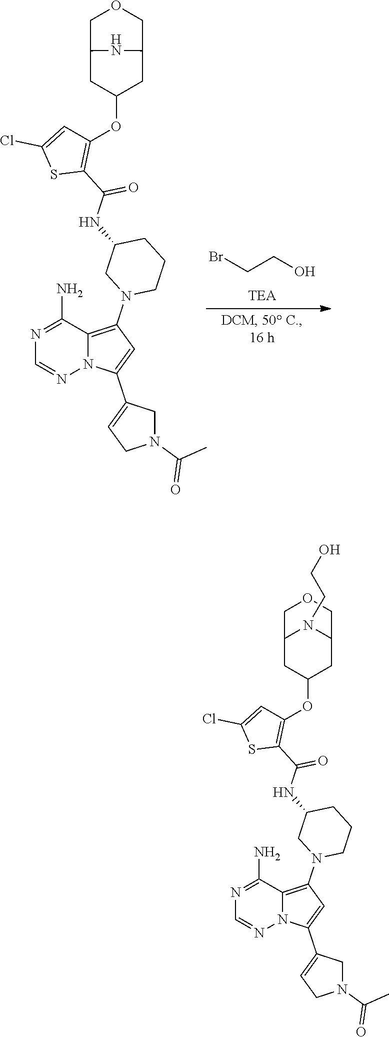

### Example 986A. tert-butyl 7-hydroxy-3-oxa-9-azabicyclo[3.3.1]nonane-9-carboxylate

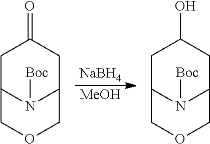

Analogous to the preparation of Example 946A, starting with tert-butyl 7-oxo-3-oxa-9-azabicyclo[3.3.1]nonane-9-carboxylate (1.0 g, 4.14 mmol) and NaBH₄ (0.314 g, 8.29 mmol) in methanol (10 ml) afforded Example 946A (940 mg, 93%) as a white solid; ¹H NMR (400 MHz, CDCl₃) δ ppm 5.51 (d, J=12.6 Hz, 1H), 4.16 (br s, 1H), 4.06-4.00 (m, 1H), 3.91-3.72 (m, 5H), 2.24-2.10 (m, 2H), 1.84 (br d, J=15.3 Hz, 2H), 1.49 (s, 9H).

### Example 986B. tert-Butyl 7-((5-chloro-2-(methoxycarbonyl)thiophen-3-yl)oxy)-3-oxa-9-azabicyclo[3.3.1]nonane-9-carboxylate

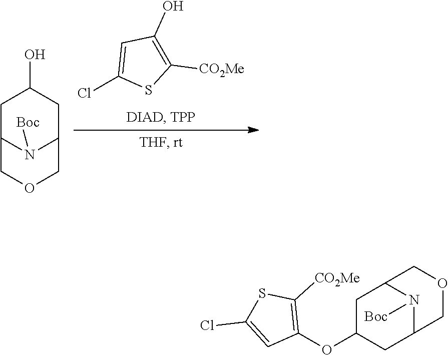

Analogous to the preparation of Example 946B, starting with methyl 5-chloro-3-hydroxythiophene-2-carboxylate (650 mg, 3.37 mmol), Example 986A (821 mg, 3.37 mmol), triphenylphosphine (1.33 g, 5.06 mmol) and DIAD (0.984 mL, 5.06 mmol) in THF (8 mL) afforded Example 986B (1.0 g, 71%). MS: [M+H]⁺=418.0; ¹H NMR (500 MHz, CDCl₃) δ ppm 7.75 (s, 1H), 5.94-5.86 (m, 1H), 4.77-4.71 (m, 1H), 4.65-4.56 (m, 1H), 4.37-4.13 (m, 6H), 2.77-2.69 (m, 1H), 2.51-2.36 (m, 1H), 2.08-2.04 (m, 1H), 2.00-1.90 (m, 8H), 1.83-1.70 (m, 3H).

### Example 986C. Lithium 3-((9-(tert-butoxycarbonyl)-3-oxa-9-azabicyclo[3.3.1]nonan-7-yl)oxy)-5-chlorothiophene-2-carboxylate

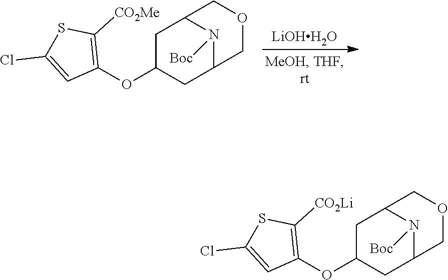

Analogous to the preparation of Example 946E, starting with Example 986B (1.0 g, 2.39 mmol) and lithium hydroxide monohydrate (0.201 g, 4.79 mmol) in THF/Methanol/water afforded Example 986C (850 mg, 87%) as a white solid. MS: [M+H]⁺=404.15; ¹H NMR (400 MHz, DMSO-d₆) δ ppm 6.77 (s, 1H), 4.08-3.92 (m, 2H), 3.82-3.71 (m, 2H), 3.59-3.42 (m, 3H), 2.27-2.14 (m, 2H), 1.69-1.55 (m, 2H), 1.46-1.41 (m, 9H).

### Example 986D. tert-Butyl 7-((2-(((R)-1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)-3-oxa-9-azabicyclo[3.3.1]nonane-9-carboxylate

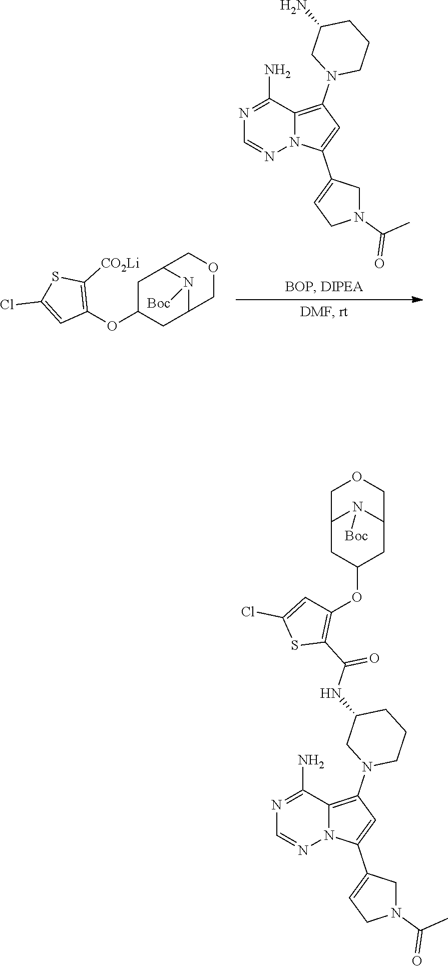

Analogous to the preparation of Example 977C, starting with Intermediate 32, HCl salt (323 mg, 0.854 mmol), Example 986C (350 mg, 0.854 mmol), BOP (453 mg, 1.03 mmol) and DIEA (0.448 mL, 2.56 mmol) in DMF (3 mL) afforded Example 986D as a yellow solid (290 mg, 47%). MS: [M+H]⁺=727.25; ¹H NMR (500 MHz, DMSO-d₆) δ ppm 8.01 (br s, 1H), 7.86 (d, J=1=2.7 Hz, H), 7.65-7.44 (m, 1H), 7.39 (br d, J=3.2 Hz, 1H), 6.80-6.62 (m, 3H), 5.76 (s, 1H), 5.42 (br s, 1H), 4.68 (br s, 1H), 4.48 (br d, J=9.5 Hz, 2H), 4.26 (br s, 1H), 4.16 (br s, 1H), 4.02 (br d, J=19.7 Hz, 2H), 3.88 (br dd, J=11.4, 19.1 Hz, 2H), 3.52 (br d, J=11.3 Hz, 2H), 3.06-2.91 (m, 2H), 2.46-2.26 (nm, 3H), 2.03 (d, J=19.4 Hz, 3H), 1.82 (br s, 2H), 1.76 (br s, 1H), 1.63 (br s, 3H), 1.33 (br s, 9H).

### Example 986E

### 3-((3-Oxa-9-azabicyclo[3.3.1]nonan-7-yl)oxy)-N—((R)-1-(7-(1-acetyl-2,5-dihydro-1H-pyrrol-3-yl)-4-aminopyrrolo[2,1-f][1,2,4]triazin-5-yl)piperidin-3-yl)-5-chlorothiophene-2-carboxamide

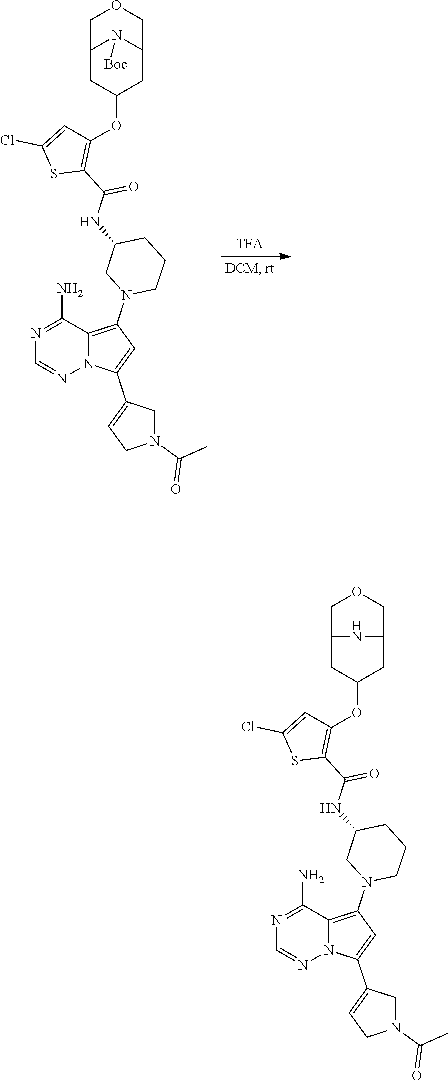 [SMILES: CC(=O)N1CC=C(c2cc(N3CCC[C@@H](NC(=O)c4sc(Cl)cc4OC4CC5COCC(C4)N5C(=O)OC(C)(C)C)C3)c3c(N)ncnn23)C1 ; CC(=O)N1CC=C(c2cc(N3CCC[C@@H](NC(=O)c4sc(Cl)cc4OC4CC5COCC(C4)N5)C3)c3c(N)ncnn23)C1]

Analogous to the preparation of Example 977F, reaction of Example 986D (290 mg, 0.399 mmol) and TFA (0.614 mL, 7.97 mmol) in DCM (5 mL) afforded Example 986E (250 mg, 100%) as a pale-yellow solid. LC-MS Method E: RT=1.34 min, [M+H]⁺=627.2; LC-MS Method F: RT=0.99 min, [M+H]⁺=627.3; ¹H NMR (400 MHz, DMSO-d₆) δ ppm 8.08-7.95 (m, 1H), 7.89-7.84 (m, 1H), 7.55-7.42 (m, 1H), 7.34-7.27 (m, 1H), 6.84-6.64 (m, 3H), 5.41-5.30 (m, 1H), 4.73-4.64 (m, 1H), 4.55-4.43 (m, 2H), 4.30-4.22 (m, 1H), 4.20-4.12 (m, 1H), 3.80-3.71 (m, 2H), 3.67-3.59 (m, 2H), 3.19-3.11 (m, 2H), 3.04-2.90 (m, 4H), 2.37-2.26 (m, 4H), 2.03 (d, J=16.0 Hz, 3H), 1.89-1.70 (m, 5H).

### Example 986

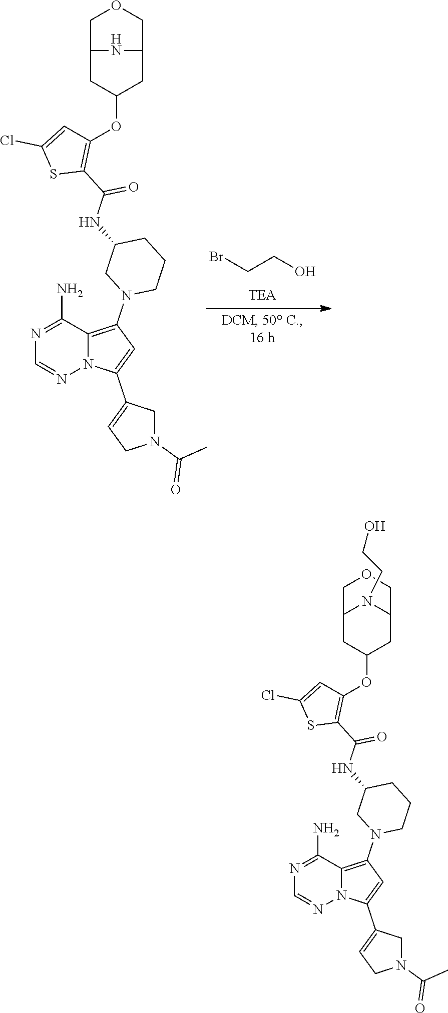

To a stirred solution of Example 986E (50 mg, 0.067 mmol) in DCM (3 mL) was added TEA (0.028 mL, 0.202 mmol) and 2-bromoethan-1-ol (11.0 mg, 0.088 mmol). The reaction mixture was heated at 50° C. for 16 h. The mixture was filtered, concentrated and the residue was purified by preparative HPLC to afford Example 986 (14.3 mg, 32%) as a pale-yellow solid. LC-MS Method E: RT=1.39 min, [M+H]⁺=671.2; LC-MS Method F: RT=0.97 min, [M+H]⁺=671.3; ¹H NMR (400 MHz, DMSO-d₆) δ ppm 7.86 (d, J=2.1 Hz, 1H), 7.66-7.49 (m, 1H), 7.32 (d, J=1.5 Hz, 1H), 6.80-6.65 (m, 2H), 5.30 (s, 1H), 4.69 (br s, 1H), 4.49 (br d, J=7.8 Hz, 2H), 4.33-4.24 (m, 2H), 4.16 (br s, 1H), 3.75-3.62 (m, 4H), 3.22-3.07 (m, 3H), 2.92 (br s, 3H), 2.88-2.68 (m, 5H), 2.08-1.99 (m, 6H), 1.96-1.78 (m, 6H).

Examples 987-988 in Table 66 were prepared using the same procedure as shown in Example 986.

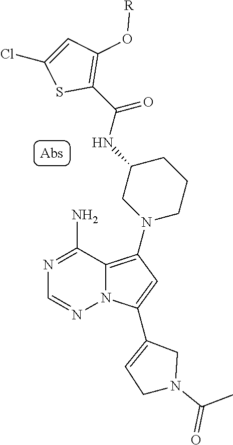

TABLE 66

| | | | | LCMS | |
|---|---|---|---|---|---|
| | | | LCMS | Method, | |
| Example | R | Name | (M+H)+ | RT (min.) | 1H NMR |
| 987 | | N-((R)-1-(7-(1-acetyl-2,5-dihydro-1H- | 641.3 | E: 1.38 | (400 MHZ, DMSO-d6) 8 ppm 8.09-7.94 (m, 1H), 7.87 (d, ] = 1.6 |
| | | pyrrol-3-y1)-4-aminopyrrolo[2,1- | | F: 1.03 | Hz, 1H), 7.54-7.38 (m, 1H), 7.32 (s, 1H), 6.71 (br d, J = 12.1 Hz, |
| | | [][1,2,4]triazin-5-yl)piperidin-3-y1)-5- | | | 3H), 4.77-4.65 (m, 2H), 4.54-4.43 (m, 3H), 4.32-4.22 (m, 1H), |
| | | chloro-3-((1-isopropylazepan-4- | | | 4.19-4.04 (m, 1H), 3.20-3.06 (m, 4H), 2.98-2.74 (m, 5H), 2.03 |
| | | yl)oxy)thiophene-2-carboxamide | | | (d, J = 16.0 Hz, 3H), 1.89-1.74 (m, 5H), 1.71-1.45 (m, 4H), 0.92- |
| | | | | | 0.81 (m, 6H). |
| Example | R | Name | LCMS | LCMS | IH NMR |
| | | | (M+H)+ | Method, | |
| | | | | RT (min.) | |
| 988 | | 3-(((IR,3s.5S)-8-oxabicyclo[3.2.1]octan-3- | 612.3 | E: 1.68 | (400 MHZ, DMSO-d6) 8 ppm 7.87 (d, J = 2.0 Hz, 1H), 7.50 (d, J |
| | | y1)oxy)-N-((R)-1-(7-(1-acetyl-2,5-dihydro- | | F: 1.38 | = 1.0 Hz, 1H), 7.39 (s, 1H), 6.80-6.72 (m, 2H), 4.82-4.75 (m, |
| | | 1H-pyrrol-3-y1)-4-aminopyrrolo[2,1- | | | 1H), 4.68 (br s, 1H), 4.50-4.45 (m, 2H), 4.38 (br s, 2H), 4.29- |
| | | f][1,2,4]triazin-5-y1)piperidin-3-y1)-5- | | | 4.25 (m, 1H), 4.14 (br s, IH), 3.10 (br s, 1H), 2.95 (br s, IH), |
| | | chlorothiophene-2-carboxamide | | | 2.81 (s, 1H), 2.08 (s, 3H), 2.03 (d. J = 16.3 Hz, 3H), 1.91-1.77 |
| | | | | | (m, 7H), 1.69-1.48 (m, 3H). |

### Example 989

### Preparation of N—((R)-1-(7-(6-(1H-tetrazol-1-yl)pyridin-3-yl)-4-aminopyrrolo[2,1-f][1,2,4]triazin-5-yl)piperidin-3-yl)-5-chloro-3-((2-isopropyl-2-azabicyclo[2.2.2]octan-5-yl)oxy)thiophene-2-carboxamide

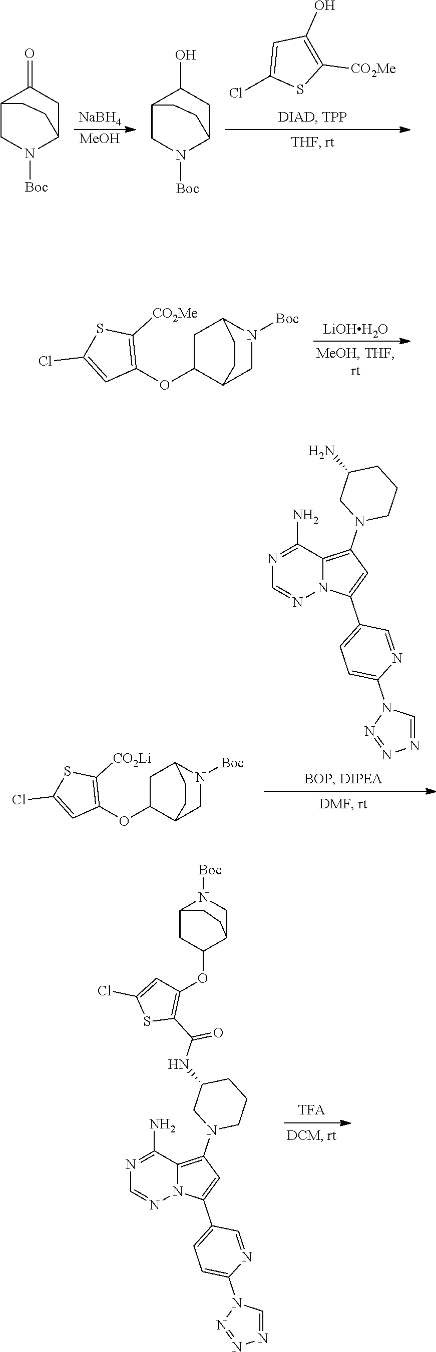

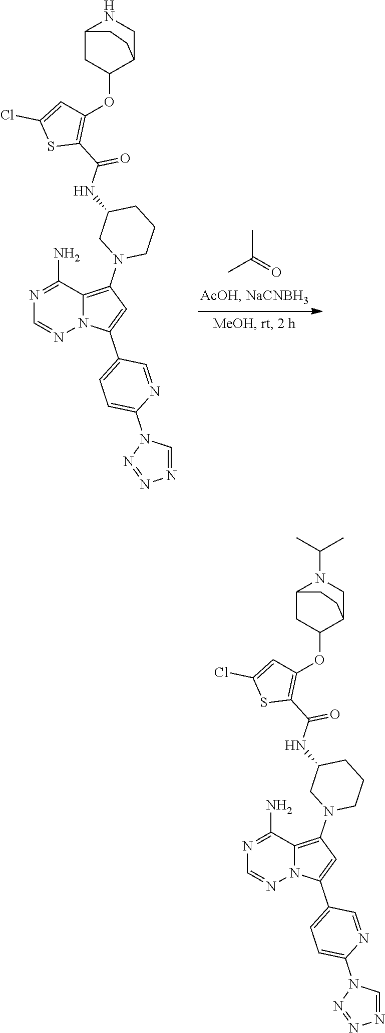

### Example 989A. tert-Butyl 5-hydroxy-2-azabicyclo[2.2.2]octane-2-carboxylate

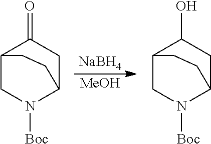

Analogous to the preparation of Example 946A, starting with tert-butyl 5-oxo-2-azabicyclo[2.2.2]octane-2-carboxylate (5.0 g, 22.2 mmol) and NaBH₄ (1.68 g, 44.4 mmol) in methanol (60 mL) afforded Example 989A (5.0 g, 99%) as a colorless viscous liquid. ¹H NMR (400 MHz, CHCl₃-d) δ ppm 4.22-3.90 (m, 2H), 3.77-3.65 (m, 1H), 3.46-3.16 (m, 2H), 2.44-2.24 (m, 1H), 2.18-2.03 (m, 1H), 1.99-1.88 (m, 1H), 1.87-1.78 (m, 1H), 1.78-1.67 (m, 1H), 1.60-1.58 (m, 1H), 1.56-1.50 (m, 1H), 1.50-1.45 (m, 9H).

### Example 989B. tert-Butyl 5-((5-chloro-2-(methoxycarbonyl)thiophen-3-yl)oxy)-2-azabicyclo[2.2.2]octane-2-carboxylate

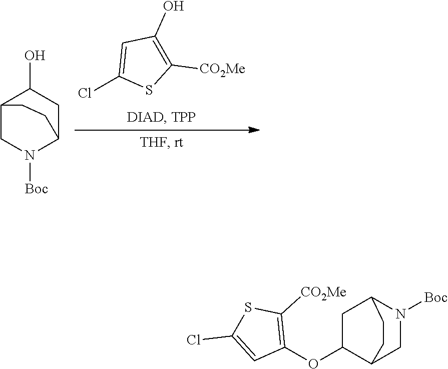

Analogous to the preparation of Example 946B, starting with methyl 5-chloro-3-hydroxythiophene-2-carboxylate (3.85 g, 20.0 mmol), Example 989A (5.00 g, 22.0 mmol), triphenylphosphine (7.86 g, 30.0 mmol) and DIAD (5.83 mL, 30.0 mmol) in THF (50 mL) afforded Example 989B as diastereomeric mixture which was purified by column chromatography to obtain two diastereomeric mixtures (1&2). Diastereomeric mixture 2 was separated by SFC to obtain two chiral isomers Iso-1 and Iso-2, mentioned below. Iso-2 was taken for the next step. Diastereomeric Mixture-1: Yellow thick liquid; MS: [M−^{t}Bu]⁺=346.15; ¹H NMR (400 MHz, CHCl₃-d) δ ppm 6.73-6.60 (m, 1H), 4.67-4.52 (m, 1H), 4.22-3.94 (m, 1H), 3.85-3.76 (m, 3H), 3.60-3.41 (m, 1H), 3.39-3.19 (m, 1H), 2.45-2.18 (m, 3H), 1.96-1.72 (m, 3H), 1.51-1.44 (m, 9H), 1.35-1.23 (m, 1H). Diastereomeric Mixture-2: Yellow thick liquid; [M−^{t}Bu]⁺=346.10. Diastereomeric mixture-2 was separated by SFC [Preparative SFC Conditions: Column/dimensions: Chiralcel ADH-2(250×4.6) mm, 5 μm; % CO₂: 85%, % Cosolvent: 15% of NH₄OAc in ACN:MeOH; Total Flow: 4 g/min; Back Pressure: 100bar; Temperature: 40° C.; U V: 254 nm] to get two chiral isomers (Iso-1 and Iso-2). First eluting isomer (Iso-1): 1.7 g, 21%, yellow thick liquid; SFC Method: RT=2.54 min; 100% ee; MS: [M−^{t}Bu]⁺=346.15; ¹H NMR (400 MHz, CHCl₃-d) δ ppm 6.78-6.57 (m, 1H), 4.58-4.36 (m, 1H), 4.25-3.99 (m, 1H), 3.91-3.75 (m, 4H), 3.36-3.20 (m, 1H), 2.32-2.10 (m, 3H), 2.04-1.76 (m, 3H), 1.58-1.40 (m, 10H). Second eluting isomer (Iso-2): 2.0 g, 25%, yellow thick liquid; SFC Method: RT=3.03 min; 95.6% ee; MS: [M−^{t}Bu]⁺=346.10; ¹H NMR (400 MHz, CHCl₃-d) δ ppm 6.68 (s, 1H), 4.53-4.47 (m, 1H), 4.20 (br s, 1H), 3.90-3.81 (m, 3H), 3.34-3.25 (m, 1H), 2.27-2.10 (m, 3H), 2.05-1.76 (m, 3H), 1.65 (br s, 1H), 1.60-1.47 (m, 8H), 1.38-1.24 (m, 2H).

### Example 989C. Lithium 3-((2-(tert-butoxycarbonyl)-2-azabicyclo[2.2.2]octan-5-yl)oxy)-5-chlorothiophene-2-carboxylate

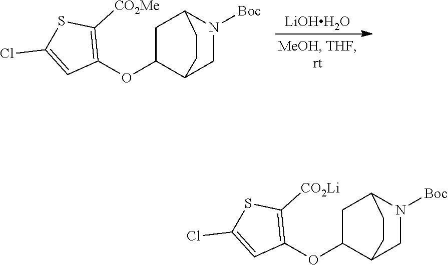

Analogous to the preparation of Example 946E, starting with Example 989B (Iso-2, 2.0 g, 4.98 mmol) and lithium hydroxide monohydrate (0.418 g, 9.95 mmol) in THF/methanol/water afforded Example 989C (1.8 g, 92%) as a white solid. MS: [M−^{t}Bu]⁺=330.15; ¹H NMR (400 MHz, DMSO-d₆) δ ppm 6.77 (s, 1H), 4.84-4.65 (m, 1H), 3.93 (br s, 1H), 3.70-3.58 (m, 1H), 3.10 (br t, J=12.3 Hz, 1H), 2.17-1.97 (m, 3H), 1.70-1.56 (m, 3H), 1.44-1.38 (m, 9H).

### Example 989D. tert-Butyl 5-((2-(((R)-1-(7-(6-(1H-tetrazol-1-yl)pyridin-3-yl)-4-aminopyrrolo[2,1-f][1,2,4]triazin-5-yl)piperidin-3-yl)carbamoyl)-5-chlorothiophen-3-yl)oxy)-2-azabicyclo[2.2.2]octane-2-carboxylate

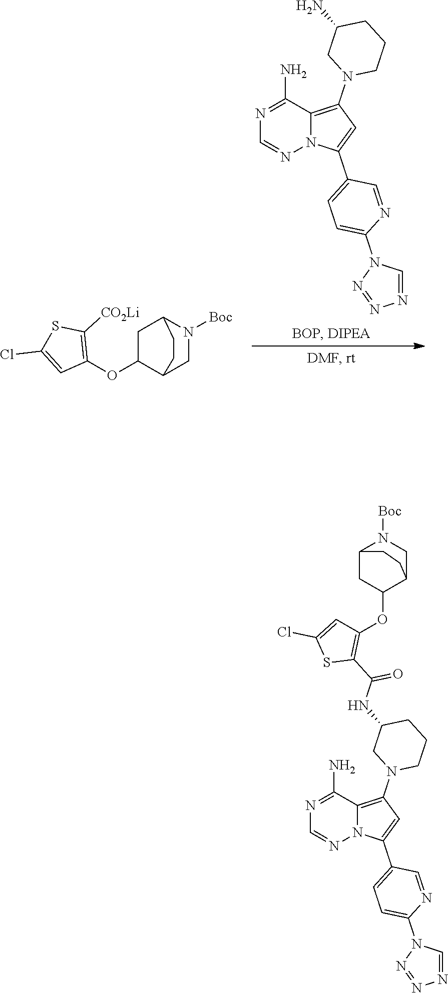

Analogous to the preparation of Example 977C, starting with (R)-7-(6-(1H-tetrazol-1-yl)pyridin-3-yl)-5-(3-aminopiperidin-1-yl)pyrrolo[2,1-f][1,2,4]triazin-4-amine.HCl (2.10 g, 5.08 mmol), Example 989C (2.0 g, 5.08 mmol), BOP (2.70 g, 6.09 mmol) and DIEA (2.66 mL, 15.2 mmol) in DMF (25 mL) afforded Example 989D (1.8 g, 47%) yellow solid. MS: [M+H]⁺=747.05; ¹H NMR (400 MHz, DMSO-d₆) δ ppm 10.23-10.13 (m, 1H), 9.33-9.27 (m, 1H), 8.95-8.84 (m, 1H), 8.15 (br d, J=9.0 Hz, 2H), 7.43-7.32 (m, 2H), 4.93-4.75 (m, 2H), 4.17 (br s, 1H), 3.98 (br s, 2H), 3.28-3.16 (m, 2H), 3.09 (br d, J=11.5 Hz, 2H), 2.48-2.23 (m, 4H), 2.03-1.83 (m, 3H), 1.65-1.63 (m, 3H), 1.32-1.20 (m, 13H).

### Example 989E. N—((R)-1-(7-(6-(1H-Tetrazol-1-yl)pyridin-3-yl)-4-aminopyrrolo[2,1-f][1,2,4]triazin-5-yl)piperidin-3-yl)-3-((2-azabicyclo[2.2.2]octan-5-yl)oxy)-5-chlorothiophene-2-carboxamide.TFA

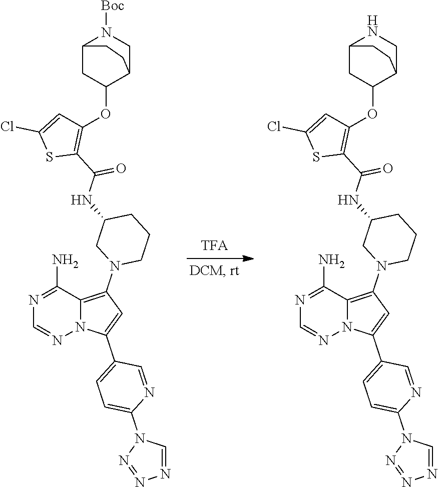 [SMILES: CC(C)(C)OC(=O)N1CC2CCC1CC2Oc1cc(Cl)sc1C(=O)N[C@@H]1CCCN(c2cc(-c3ccc(-n4cnnn4)nc3)n3ncnc(N)c23)C1 ; Nc1ncnn2c(-c3ccc(-n4cnnn4)nc3)cc(N3CCC[C@@H](NC(=O)c4sc(Cl)cc4OC4CC5CCC4CN5)C3)c12]

Analogous to the preparation of Example 977F, starting with Example 989D (1.6 g, 2.14 mmol) and TFA (2.47 mL, 32.1 mmol) in DCM (15 mL) afforded Example 989E (1.6 g, 98%) as a brown solid. MS: [M+H]⁺=647.40; ¹H NMR (400 MHz, DMSO-d₆) δ ppm 10.22-10.19 (m, 1H), 9.30-9.25 (m, 1H), 8.90-8.84 (m, 2H), 8.21-8.16 (m, 1H), 8.06-8.00 (m, 1H), 4.82-4.77 (m, 1H), 4.22-4.15 (m, 2H), 3.36-3.25 (m, 3H), 3.22-3.10 (m, 3H), 2.87-2.75 (m, 2H), 2.02-1.96 (m, 1H), 1.87-1.72 (m, 8H), 1.63-1.50 (m, 4H).

### Example 989

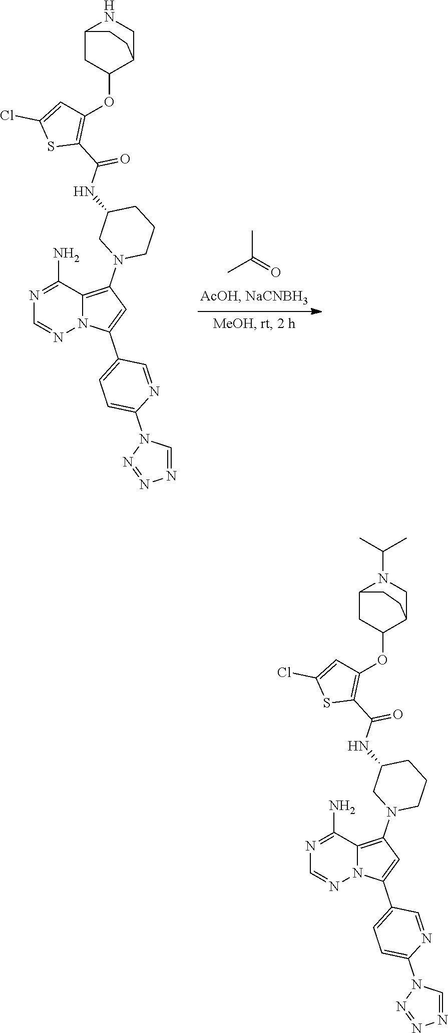

Analogous to the preparation of Example 977, starting with Example 44E (500 mg, 0.657 mmol), acetone (191 mg, 3.28 mmol), acetic acid (0.056 mL, 0.985 mmol) and sodium cyanoborohydride (83 mg, 1.31 mmol) in methanol afforded the crude product, which was purified by SFC [Preparative SFC Conditions: Column/dimensions: Princeton SFC Diol (250*4.6 mm 5μ); % CO₂: 70%, % Cosolvent: 30% of 0.2% Ammonia in methanol, Flow: 3 mL/min; Back Pressure: 100 bar; Temperature 30° C.: UV: 220 nm] to afford Example 989 (290 mg, 20%) as an off-white solid. MAS: [M+¹H]⁺=689.5; HPLC Method G: RT=6.28 min; HPLC Method H: RT=5.18 min; ¹H NMR (500 MHz, DMSO-d₆) δ ppm 10.21 (s, 1H), 9.30 (d, J=2.0 Hz, 1H), 8.89 (dd, J=2.3, 8.7 Hz, 1H), 8.16 (d, J=8.7 Hz, 2H), 7.93 (s, 1H), 7.43 (br s, 1H), 7.38-7.27 (m, 2H), 6.85 (br s, 1H), 4.72 (br d, J=9.0 Hz, 1H), 4.19 (br s, 1H), 3.03 (br s, 1H), 2.95-2.76 (m, 4H), 2.59-2.53 (m, 2H), 2.16-2.02 (m, 1H), 1.96 (br s, 2H), 1.87 (br s, 2H), 1.80-1.58 (m, 4H), 1.56 (br s, 2H), 1.30 (br t, J=9.3 Hz, 1H), 0.82 (br s, 6H).

### Example 990

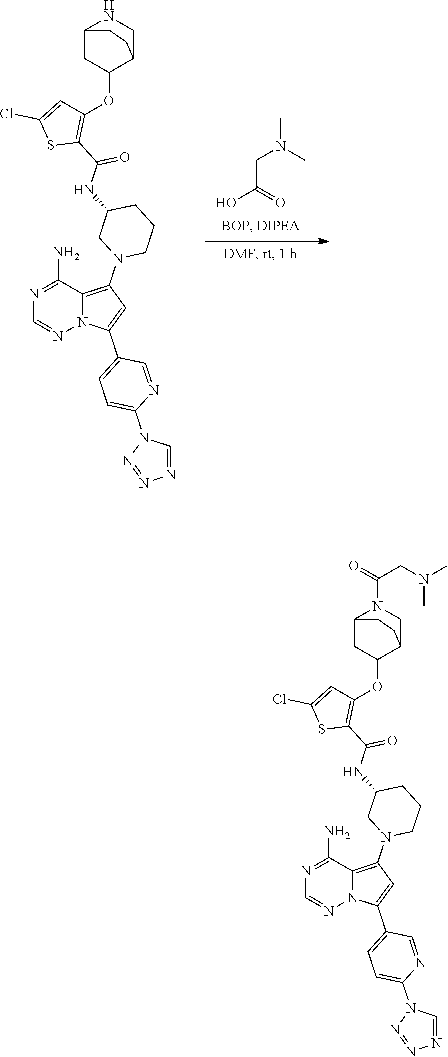

Analogous to the preparation of Example 978A, starting with Example 989E (35 mg, 0.054 mmol), dimethylglycine (5.6 mg, 0.054 mmol), BOP (28.7 mg, 0.065 mmol) and DIEA (0.028 mL, 0.162 mmol) in DMF (1 mL) afforded Example 990 (14.8 mg, 37%) as a pale-yellow solid. LC-MS Method E: RT=1.66 min, [M+H]⁺=732.2, LC-MS Method F: RT=1.10 min, [M+H]⁺=732.3; ¹H NMR (400 MHz, DMSO-d₆) δ ppm 10.24-10.12 (m, 1H), 9.37-9.27 (m, 1H), 8.94-8.85 (m, 1H), 8.21-8.14 (m, 1H), 7.99-7.81 (m, 1H), 7.40-7.22 (m, 2H), 4.89-4.79 (m, 1H), 4.30-4.07 (m, 4H), 3.84-3.67 (m, 2H), 2.84-2.75 (m, 9H), 1.99-1.44 (m, 10H), 1.32-1.20 (m, 2H).

### Example 991

### Preparation of N—((R)-1-(4-amino-7-(1-(2,2-difluoroethyl)-6-oxo-1,6-dihydropyridin-3-yl)pyrrolo[2,1-f][1,2,4]triazin-5-yl)piperidin-3-yl)-5-chloro-3-((2-methyl-2-azabicyclo[2.2.2]octan-5-yl)oxy)thiophene-2-carboxamide

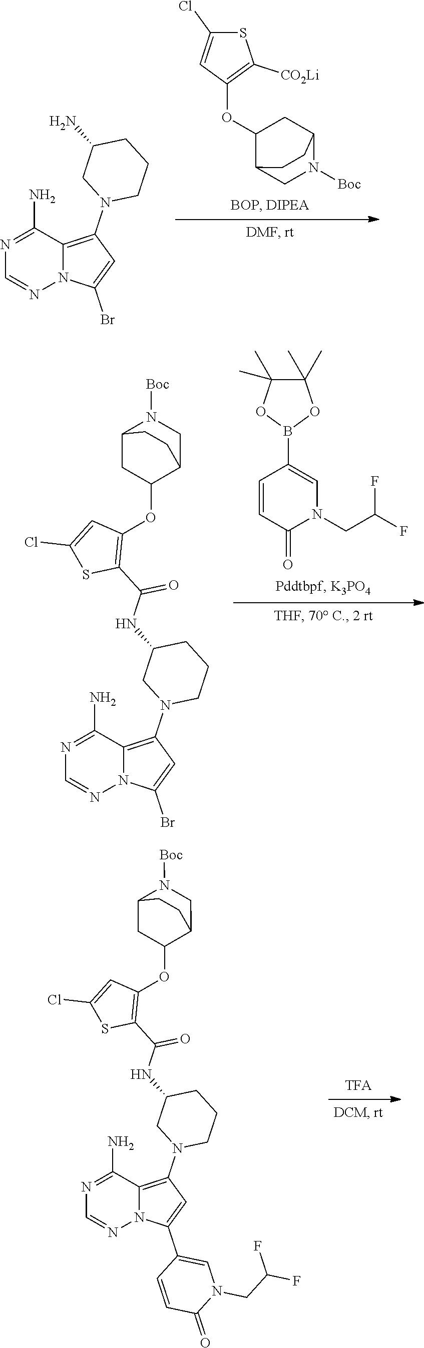

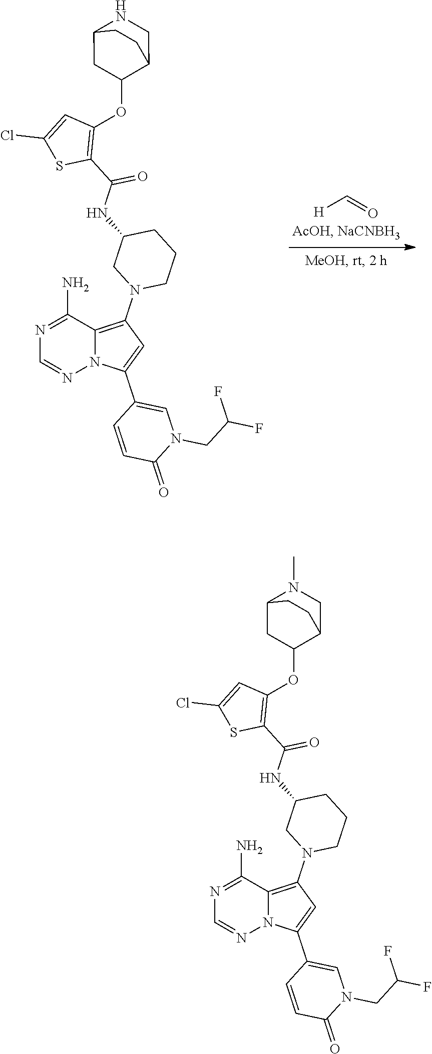 [SMILES: Nc1ncnn2c(-c3ccc(=O)n(CC(F)F)c3)cc(N3CCC[C@@H](NC(=O)c4sc(Cl)cc4OC4CC5CCC4CN5)C3)c12 ; C=O ; CN1CC2CCC1CC2Oc1cc(Cl)sc1C(=O)N[C@@H]1CCCN(c2cc(-c3ccc(=O)n(CC(F)F)c3)n3ncnc(N)c23)C1]

### Example 991A. tert-Butyl 5-((2-(((R)-1-(4-amino-7-bromopyrrolo[2,1-f][1,2,4]triazin-5-yl)piperidin-3-yl)carbamoyl)-5-chlorothiophen-3-yl)oxy)-2-azabicyclo[2.2.2]octane-2-carboxylate

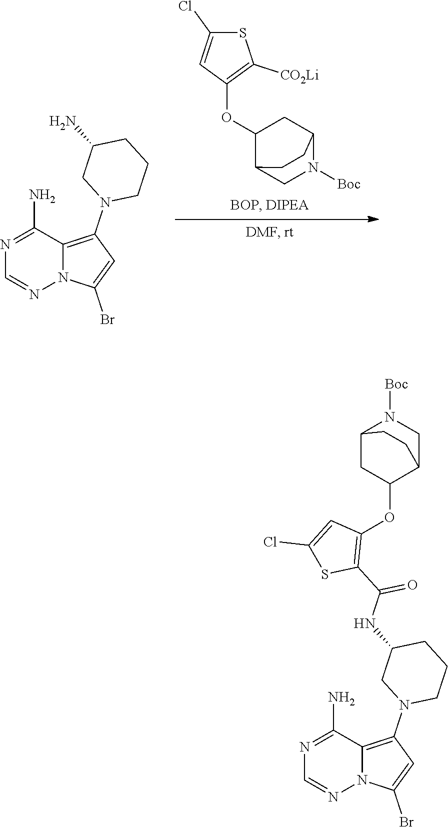

Analogous to the preparation of Example 977C, starting with Intermediate 61, TFA (572 mg, 1.35 mmol), Example 985C (530 mg, 1.35 mmol), BOP (714 mg, 1.62 mmol) and DIEA (0.705 mL, 4.04 mmol) in DMF (5 mL) afforded Example 991A (350 mg, 38%) as a yellow solid. MS: [M+H]⁺=682.00; ¹H NMR (400 MHz, DMSO-d₆) δ ppm 8.12-7.99 (m, 1H), 7.82 (s, 1H), 7.35 (d, J=15.0 Hz, 1H), 7.25-7.11 (m, 1H), 6.82 (s, 1H), 6.74 (s, 1H), 4.86-4.77 (m, 1H), 4.13 (br s, 1H), 3.97 (br d, J=14.0 Hz, 1H), 3.49 (br s, 1H), 3.27-3.15 (m, 1H), 2.98 (br s, 1H), 2.41-2.36 (m, 1H), 2.24 (br s, 1H), 1.93 (br s, 1H), 1.77-1.61 (m, 5H), 1.55-1.46 (m, 2H), 1.43-1.24 (m, 1H), 0.89-0.81 (m, 2H).

### Example 991B. tert-Butyl 5-((2-(((R)-1-(4-amino-7-(1-(2,2-difluoroethyl)-6-oxo-1,6-dihydropyridin-3-yl)pyrrolo[2,1-f][1,2,4]triazin-5-yl)piperidin-3-yl)carbamoyl)-5-chlorothiophen-3-yl)oxy)-2-azabicyclo[2.2.2]octane-2-carboxylate

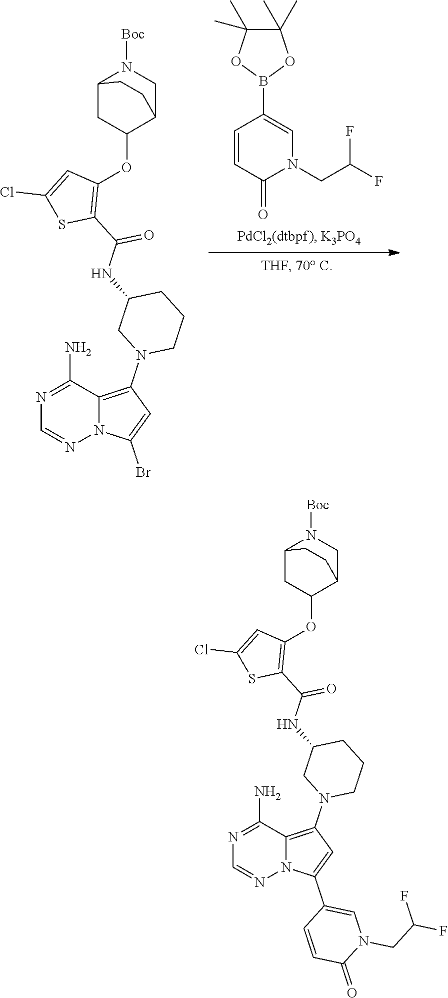 [SMILES: CC(C)(C)OC(=O)N1CC2CCC1CC2Oc1cc(Cl)sc1C(=O)N[C@@H]1CCCN(c2cc(Br)n3ncnc(N)c23)C1 ; CC1(C)OB(c2ccc(=O)n(CC(F)F)c2)OC1(C)C ; CC(C)(C)OC(=O)N1CC2CCC1CC2Oc1cc(Cl)sc1C(=O)N[C@@H]1CCCN(c2cc(-c3ccc(=O)n(CC(F)F)c3)n3ncnc(N)c23)C1]

Analogous to the preparation of Example 970E, starting with Example 991A (120 mg, 0.176 mmol), 1-(2,2-difluoroethyl)-5-(4,4,5,5-tetramethyl-1,3,2-dioxaborolan-2-yl)pyridin-2(1H)-one (100 mg, 0.352 mmol), 2M potassium phosphate (0.176 mL, 0.352 mmol) and [1,1′-bis(di-tert-butylphosphino)ferrocene]dichloropalladium(II) (6.9 mg, 10.6 mmol) in THF (3 mL) afforded Example 991B (120 mg, 90%) as a yellow solid. MS: [M+H]⁺=759.0; ¹H NMR (500 MHz, DMSO-d₆) δ ppm 7.82 (d, J=4.3 Hz, 1H), 7.44-7.32 (m, 1H), 6.99-6.97 (m, 1H), 6.90-6.86 (m, 1H), 6.63-6.53 (m, 1H), 6.36 (br s, 1H), 4.44 (br d, J=11.0 Hz, 2H), 4.37 (br d, J=4.0 Hz, 1H), 3.94 (s, 1H), 2.65 (br d, J=1.8 Hz, 1H), 2.56-2.52 (m, 2H), 2.48-2.29 (m, 2H), 1.99 (s, 1H), 1.69 (br s, 5H), 1.49 (br s, 2H), 1.43-1.27 (m, 9H), 1.25 (br s, 1H), 1.22-1.12 (m, 1H), 1.07 (s, 2H), 0.88-0.81 (m, 1H).

### Example 991C

### Preparation of 3-((2-azabicyclo[2.2.2]octan-5-yl)oxy)-N—((R)-1-(4-amino-7-(1-(2,2-difluoroethyl)-6-oxo-1,6-dihydropyridin-3-yl)pyrrolo[2,1-f][1,2,4]triazin-5-yl)piperidin-3-yl)-5-chlorothiophene-2-carboxamide.TFA

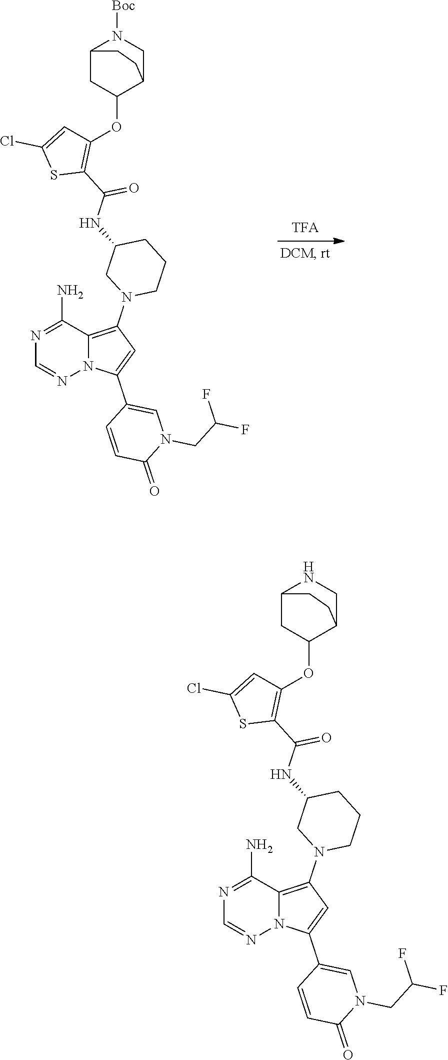 [SMILES: CC(C)(C)OC(=O)N1CC2CCC1CC2Oc1cc(Cl)sc1C(=O)N[C@@H]1CCCN(c2cc(-c3ccc(=O)n(CC(F)F)c3)n3ncnc(N)c23)C1 ; Nc1ncnn2c(-c3ccc(=O)n(CC(F)F)c3)cc(N3CCC[C@@H](NC(=O)c4sc(Cl)cc4OC4CC5CCC4CN5)C3)c12]

Analogous to the preparation of Example 977F, starting with Example 991B (120 mg, 0.158 mmol) and TFA (0.244 mL, 3.16 mmol) in DCM (2 mL) afforded Example 991C (80 mg, 72%) as a pale-yellow solid. LC-MS Method E: RT=1.64 min, [M+H]⁺=659.2; LC-MS Method F: RT=0.95 min, [M+H]⁺=659.3; ¹H NMR (400 MHz, DMSO-d₆) δ ppm 8.50 (d, J=2.3 Hz, 1H), 8.10 (dd, J=2.5, 9.6 Hz, 1H), 7.82 (s, 1H), 7.41-7.25 (m, 2H), 6.90 (s, 1H), 6.57 (d, J=9.5 Hz, 1H), 6.36 (tt, J=16.9, 62.4 Hz, 1H), 4.84-4.74 (m, 2H), 4.46 (dt, J=3.6, 14.8 Hz, 3H), 4.16 (br s, 1H), 3.19 (br d, J=10.8 Hz, 3H), 3.05-2.96 (m, 2H), 2.77 (br s, 1H), 2.31-2.08 (m, 2H), 2.00-1.43 (m, 8H).

### Example 991

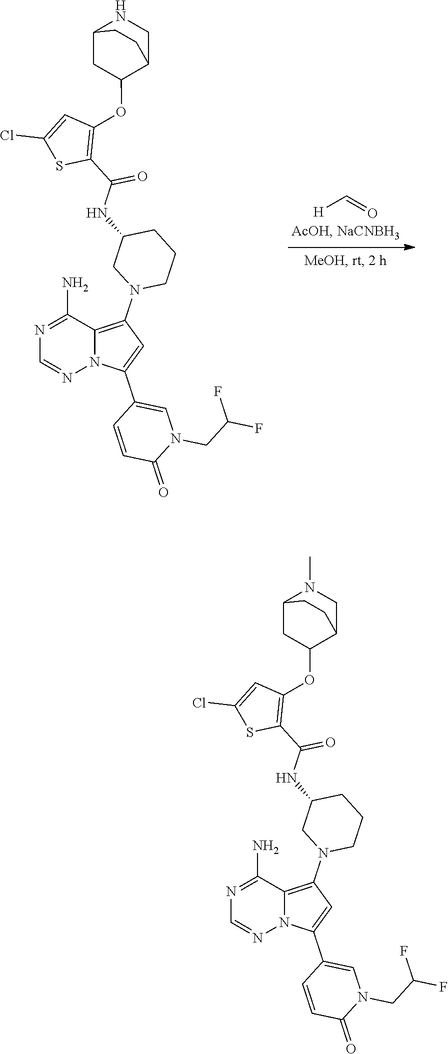 [SMILES: Nc1ncnn2c(-c3ccc(=O)n(CC(F)F)c3)cc(N3CCC[C@@H](NC(=O)c4sc(Cl)cc4OC4CC5CCC4CN5)C3)c12 ; C=O ; CN1CC2CCC1CC2Oc1cc(Cl)sc1C(=O)N[C@@H]1CCCN(c2cc(-c3ccc(=O)n(CC(F)F)c3)n3ncnc(N)c23)C1]

Analogous to the preparation of Example 977, starting with Example 991C (50 mg, 0.065 mmol), formaldehyde (26.2 mg, 0.323 mmol), acetic acid (5.6 μl, 0.097 mmol) and sodium cyanoborohydride (8.1 mg, 0.13 mmol) in methanol (2 mL) afforded Example 991 (12.6 mg, 29%) as pale-yellow solid. LC-MS Method E: RT=1.78 min, [M+H]⁺=673.2; LC-MS Method F: RT=0.96 min, [M+H]⁺=673.2; ¹H NMR (400 MHz, DMSO-d₆) δ ppm 8.49 (s, 1H), 8.10 (dd, J=2.4, 9.6 Hz, 1H), 7.82 (s, 1H), 7.55-7.45 (m, 1H), 7.36 (s, 1H), 6.89 (s, 1H), 6.57 (d, J=9.5 Hz, 1H), 6.52-6.20 (m, 1H), 4.75 (br d, J=9.4 Hz, 1H), 4.51-4.39 (m, 2H), 4.14 (br s, 1H), 3.06-2.90 (m, 3H), 2.82-2.72 (m, 1H), 2.32-2.16 (m, 4H), 1.98 (br s, 2H), 1.91 (s, 2H), 1.85 (br s, 4H), 1.65 (br s, 2H), 1.50 (br d, J=6.8 Hz, 2H), 1.39-1.12 (m, 2H).

Example 992-993 in Table 67 were prepared using the same procedure as shown in Example 991. Analogous to the synthesis of Example 989B, total 4 stereoisomers (Intermediate 3) were separated in second step. Isomer 3 (Iso-3) was used to synthesize Example 992 and Isomer 1 (Iso-1) was used to synthesize Example 993. Chiral separation details of the isomers are described below.

### Intermediate 3. Ethyl 4-((2-(tert-butoxycarbonyl)-2-azabicyclo[2.2.2]octan-5-yl)oxy)-2-methylthiazole-5-carboxylate

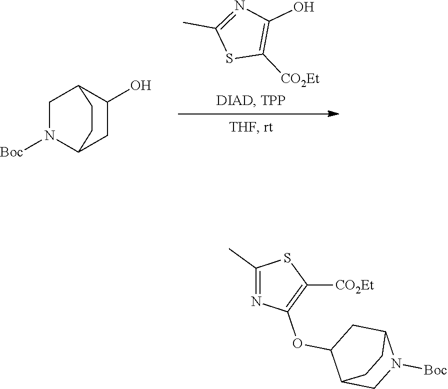 [SMILES: CCOC(=O)c1sc(C)nc1O ; CC(C)(C)OC(=O)N1CC2CCC1CC2O ; CCOC(=O)c1sc(C)nc1OC1CC2CCC1CN2C(=O)OC(C)(C)C]

Analogous to the preparation of Example 44B. Diastereomeric mixture-1 as a yellow thick liquid: MS: [M−tBu]⁺=341.15 and Diastereomeric mixture-2 as a yellow thick liquid; MS: [M−tBu]⁺=341.15 were obtained after silica-gel column chromatography. After that,

Diastereomeric mixture-1 was purified by SFC [Preparative SFC Conditions: Column/dimensions: CHIRALCEL OJ-H-2(250×4.6) mm, 5 μm; % CO₂: 90%, % Cosolvent: 10% of 0.2% DEA in MeOH: Total Flow: 3 mL/min; Back Pressure: 100 bar; Temperature: 35° C.; UV: 254 nm] to get two chiral isomers (Iso-1 and Iso-2).

First eluting isomer (Iso-1): 700 mg, 12%, yellow liquid; SFC Method: RT=3.06 min; 100% ee; MS: [M−tBu]⁺=341.15; ¹H NMR (400 MHz, CHCl₃-d) δ ppm 5.28-5.23 (m, 1H), 4.30 (q, J=7.3 Hz, 2H), 4.18-4.10 (m, 1H), 4.01 (br s, 1H), 3.50-3.36 (m, 2H), 2.63 (s, 3H), 2.49-2.31 (m, 1H), 2.30-2.18 (m, 2H), 1.96-1.70 (m, 3H), 1.49 (d, J=3.5 Hz, 9H), 1.36 (t, J=7.0 Hz, 3H).

Second eluting Isomer (Iso-2): 600 mg, 10%, yellow liquid; SFC Method: RT=4.39 min; 98.2% ee; MS. [M−tBu]⁺=341.15; ¹H NMR (400 MHz, CHCl₃-d) δ ppm 5.28-5.23 (m, 1H), 4.30 (q, J=7.21 Hz, 2H), 4.15 (br s, 1H), 4.01 (br s, 1H), 3.51-3.36 (m, 2H), 2.63 (s, 3H), 2.49-2.31 (m, 1H), 2.30-2.18 (m, 2H), 1.96-1.71 (m, 3H), 1.49 (d, J=3.5 Hz, 9H), 1.36 (t, J=7.0 Hz, 3H).

Diastereomeric mixture-2 was purified by SFC [Preparative SFC Conditions: Column/dimensions Chiralpak IG-2(250×4.6) mm, 5 μm: % CO₂: 90%, % Cosolvent 10% of 0.2% ammonia in MeOH, Total Flow: 3 mL/min; Back Pressure: 100 bar: Temperature 35° C.; UV: 254 nm] to get two chiral isomers (Iso-3 and Iso-4).

First eluting isomer (Iso-3): 750 mg, 13%, yellow liquid; SFC Method: RT=6.7 min; 100% ee, MS: [M−tBu]⁺=341.20; ¹H NMR (400 MHz, CHCl₃-d) δ ppm 5.18-5.12 (m, 1H), 5.00 (td, J=6.3, 12.5 Hz, 1H), 4.30 (q, J=7.0 Hz, 2H), 4.18 (br s, 1H), 3.90-3.79 (m, 1H), 3.36-3.26 (m, 1H), 2.63 (s, 3H), 2.33-2.21 (m, 2H), 1.98-1.85 (m, 2H), 1.83-1.66 (m, 2H), 1.51-1.46 (m, 9H), 1.39-1.33 (m, 3H).

Second eluting Isomer (Iso-4): 800 mg, 14%, yellow thick liquid; SFC Method: RT=7.41 min; 97.2% ee; MS: [M−tBu]=341.15; ¹H NMR (400 MHz, CHCl₃-d) δ ppm 5.15 (br d, J=8.5 Hz, 1H), 4.30 (q, J=7.2 Hz, 2H), 4.18 (br s, 1H), 3.90-3.79 (m, 1H), 3.35-3.26 (m, 1H), 2.63 (s, 3H), 2.33-2.21 (m, 2H), 1.98-1.85 (m, 2H), 1.84-1.65 (m, 3H), 1.51-1.46 (m, 9H), 1.39-1.33 (m, 3H).

TABLE 67

| | | | | | HPLC | |
|---|---|---|---|---|---|---|
| | | | | LCMS | Method, | |
| Example | R | R1 | Name | (M+H)+ | RT (min.) | 1HNMR |
| 992 | | Methyl | N-((R)-1-(7-(6-(1H-tetrazol-1- | 642.3 | E: 1.41 | (400 MHZ, DMSO-d6) 8 ppm 10.21 (s, IH), 9.31 (d, J = |
| (Iso-3) | | | y1)pyridin-3-y1)-4-aminopyrrolo[2,1- | | F: 0.91 | 2.3 Hz, 1H), 8.90 (dd, J = 2.4, 8.6 Hz, 1H), 8.16 (d, J = |
| | | | f][1,2,4]triazin-5-y1)piperidin-3-y1)-2- | | | 8.6 Hz, 2H), 7.94 (s, IH), 7.45 (br s, IH), 7.33 (s, 1H), |
| | | | methyl-4-((2-methyl-2- | | | 6.91 (s, 1H), 5.14 (br d, J = 9.3 Hz, 1H), 4.19 (br s, 1H), |
| | | | azabicyclo[2.2.2]octan-5- | | | 3.22 (br d, J = 3.9 Hz, 1H), 3.08-2.74 (m, 5H), 2.61 (s, |
| | | | y1)oxy )thiazole-5-carboxamide | | | 3H), 2.27 (br s, 2H), 2.08 (s, 2H), 1.91 (s, 6H), 1.69-1.28 |
| | | | | | | (m, 5H) |
| 993 | N | OH | N-((R)- 1-(4-amino-7-(6-cyano-5 .. | 647.2 | E: 1.56 | (400 MHZ, DMSO-d6) 8 ppm 9.36 (t, J = 1.5 Hz, 1H), |
| (Iso-1) | CN | | fluoropyridin-3-y1)pyrrolo[2,1- | | F: 1.18 | 8.84 (dd, J = 1.6, 11.4 Hz, 1H), 8.37 (br s, 1H), 8.01 (s, |
| | | | [][1,2,4]triazin-S-y])piperidin-3-y1)-4- | | | 1H), 7.53 (s, 1H), 7.30 (br s, 1H), 6.91 (br s, IH), 5.28- |
| | | | ((2-(2-hydroxyethyl)-2- | | | 5.13 (m, 1H), 4.38 (br s, 1H), 4.20 (br d, J = 3.1 Hz, 1H), |
| | | | azabicyclo[2.2.2]octan-5-yl)oxy)-2- | | | 3.46-3.43 (m, 2H), 3.18 (br s, IH), 3.08-3.00 (m, 1H), |
| | | | methylthiazole-5-carboxamide | | | 2.94-2.67 (m, 4H), 2.63-2.54 (m, 4H), 1.96-1.69 (m, |
| | | | | | | 6H), 1.62-1.31 (m, 3H) |

### Example 994

### Preparation of N—((R)-1-(4-amino-7-(3-fluoro-4-(1H-tetrazol-1-yl)phenyl)pyrrolo[2,1-f][1,2,4]triazin-5-yl)piperidin-3-yl)-5-chloro-3-((2-(oxetan-3-yl)-2-azabicyclo[2.2.1]heptan-5-yl)oxy)thiophene-2-carboxamide

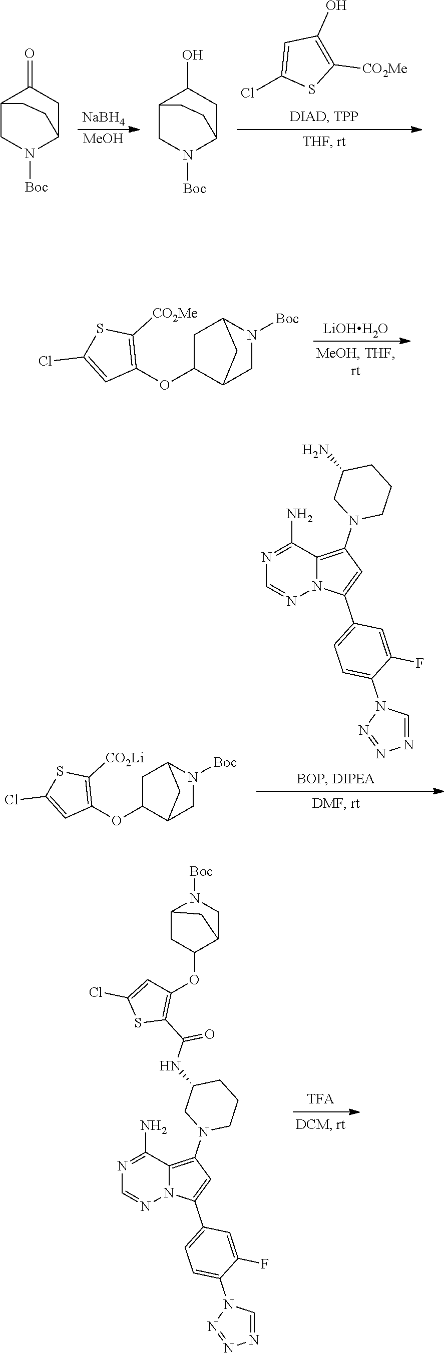

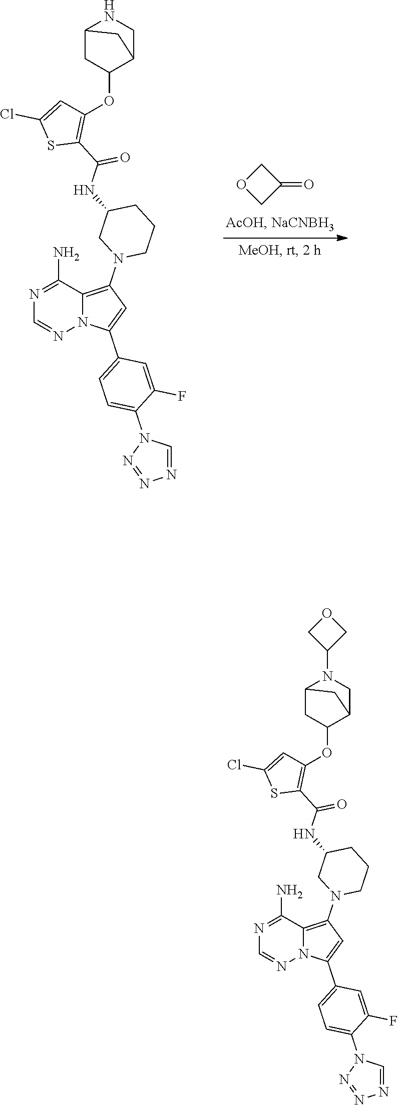 [SMILES: Nc1ncnn2c(-c3ccc(-n4cnnn4)c(F)c3)cc(N3CCC[C@@H](NC(=O)c4sc(Cl)cc4OC4CC5CC4CN5)C3)c12 ; O=C1COC1 ; Nc1ncnn2c(-c3ccc(-n4cnnn4)c(F)c3)cc(N3CCC[C@@H](NC(=O)c4sc(Cl)cc4OC4CC5CC4CN5C4COC4)C3)c12]

### Example 994A. tert-Butyl 5-hydroxy-2-azabicyclo[2.2.1]heptane-2-carboxylate

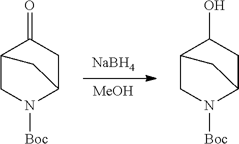 [SMILES: "MeOH" ; CC(C)(C)OC(=O)N1CC2CC1CC2=O ; "NaBH4" ; CC(C)(C)OC(=O)N1CC2CC1CC2O]

Analogous to the preparation of Example 946A, starting with tert-butyl 5-oxo-2-azabicyclo[2.2.1]heptane-2-carboxylate (2.3 g, 10.9 mmol) and NaBH₄ (0.618 g, 16.3 mmol) in methanol (20 ml) afforded Example 994A (2.2 g, 95%) as a white solid; ¹H NMR (400 MHz, CHCl₃-d) δ ppm 4.41 (br s, 1H), 4.23 (br s, 1H), 4.10 (br s, 1H), 3.70 (br t, J=10.8 Hz, 1H), 3.25-3.13 (m, 1H), 2.61 (br s, 1H), 2.07 (ddd, J=2.8, 10.0, 13.3 Hz, 1H), 1.76-1.71 (m, 1H), 1.53-1.44 (m, 1H).

### Example 994B. tert-Butyl 5-((5-chloro-2-(methoxycarbonyl)thiophen-3-yl)oxy)-2-azabicyclo[2.2.1]heptane-2-carboxylate

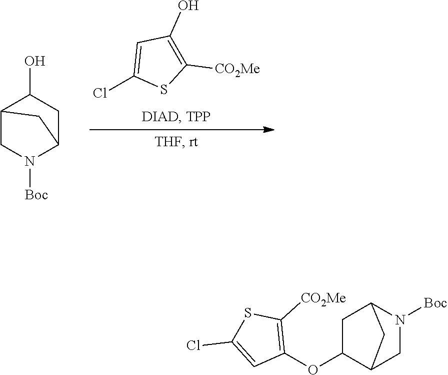 [SMILES: CC(C)(C)OC(=O)N1CC2CC1CC2O ; COC(=O)c1sc(Cl)cc1O ; COC(=O)c1sc(Cl)cc1OC1CC2CC1CN2C(=O)OC(C)(C)C]

Analogous to the preparation of Example 946B, starting with methyl 5-chloro-3-hydroxythiophene-2-carboxylate (1.0 g, 5.19 mmol), Example 994A (1.22 g, 5.71 mmol), triphenylphosphine (2.04 g, 7.79 mmol) and DIAD (1.51 mL, 7.79 mmol) in THF (10 mL) afforded Example 994B as a diastereomeric mixture. The residue was purified by SFC [Preparative SFC Conditions: Column/dimensions: Chiralpak IC-2 (250×4.6) mm, 5 μm; % CO₂: 85% % Cosolvent: 15% of 5 mM 0.2% DEA in IPA: Total Flow: 4 g/min, Back Pressure: 100 bar; Temperature: 35° C.; UV: 280 nm] to get first eluting isomer (Iso-1) (540 mg, 27%, yellow thick liquid; SFC Method: RT=5.77 min; 100% ee: MS: [M−^{t}Bu]⁺=332.10; ¹H NMR (400 MHz, DMSO-d₆) δ ppm 7.40 (s, 1H), 4.68 (br d, J=6.0 Hz, 1H), 4.13 (br d, J=13.0 Hz, 1H), 3.72 (s, 3H), 3.25-3.11 (m, 1H), 2.94 (br t, J=8.8 Hz, 1H), 2.70-2.61 (m, 1H), 2.12 (ddd, J=2.3, 6.8, 13.5 Hz, 1H), 1.76 (br t, J=8.3 Hz, 1H), 1.58 (br d, J=14.0 Hz, 2H), 1.40 (s, 9H)) and second eluting isomer (Iso-2) (640 mg, 32%, yellow liquid: SFC Method: RT=6.56 min; 99% ee; MS: [M−^{t}Bu]⁺=332.15; ¹H NMR (400 MHz, DMSO-d₆) δ ppm 7.40 (s, 1H), 4.68 (br d, J=6.0 Hz, 1H), 4.13 (br d, J=11.5 Hz, 1H), 3.72 (s, 3H), 3.14 (br d, J=9.5 Hz, 1H), 2.94 (br t, J=9.0 Hz, 1H), 2.67-2.63 (m, 1H), 2.11 (br dd, J=6.5, 13.0 Hz, 1H), 1.76 (br s, 1H), 1.58 (br d, J=14.0 Hz, 2H), 1.40 (s, 9H)). Iso-2 was taken for next step.

### Example 994C. Lithium 3-((2-(tert-butoxycarbonyl)-2-azabicyclo[2.2.1]heptan-5-yl)oxy)-5-chlorothiophene-2-carboxylate

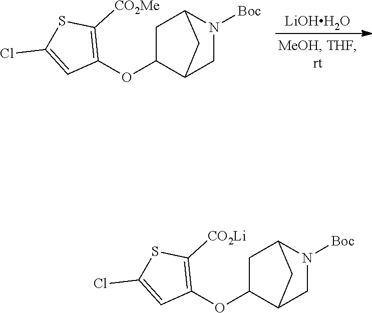

Analogous to the preparation of Example 946E, starting with Example 994B (Iso-2, 640 mg, 1.65 mmol) and lithium hydroxide monohydrate (138 mg, 3.30 mmol) in THF/methanol/water afforded Example 994C (600 mg, 96%) as a white solid. [M−tBu]⁺=318.1; ¹H NMR (400 MHz, DMSO-d₆) δ ppm 6.84 (s, 1H), 4.73 (br d, J=6.5 Hz, 1H), 4.06 (br d, J=12.5 Hz, 1H), 3.16-3.06 (m, 1H), 2.87-2.72 (m, 1H), 2.59-2.53 (m, 1H), 1.96-1.82 (m, 2H), 1.65-1.57 (m, 1H), 1.54-1.36 (m, 101H).

### Example 994D. tert-Butyl 5-((2-(((R)-1-(4-amino-7-(3-fluoro-4-(1H-tetrazol-1-yl)phenyl)pyrrolo[2,1-f][1,2,4]triazin-5-yl)piperidin-3-yl)carbamoyl)-5-chlorothiophen-3-yl)oxy)-2-azabicyclo[2.2.1]heptane-2-carboxylate

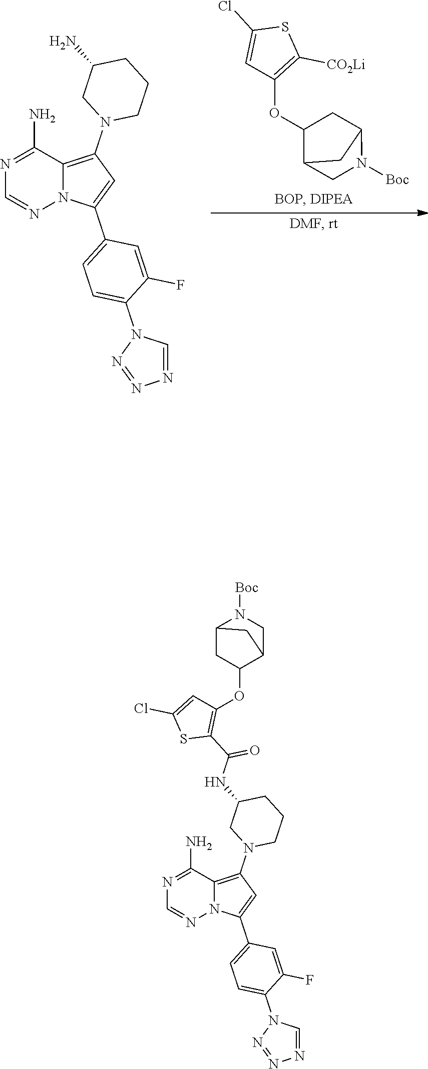

Analogous to the preparation of Example 977C, starting with (R)-5-(3-aminopiperidin-1-yl)-7-(3-fluoro-4-(1H-tetrazol-1-yl)phenyl)pyrrolo[2,1-f][1,2,4]triazin-4-amine. HCl (227 mg, 0.527 mmol), Example 994C (200 mg, 0.527 mmol), BOP (280 mg, 0.632 mmol) and DIEA (0.276 mL, 1.580 mmol) in DMF (5 mL) afforded Example 994D (340 mg, 86%) yellow solid. MS: [M+H]⁺=750.50; ¹H NMR (400 MHz, DMSO-d₆) δ ppm 9.97-9.85 (m, 1H), 8.48-8.34 (m, 1H), 8.30-8.18 (m, 1H), 8.02-7.89 (m, 2H), 7.38-7.31 (m, 1H), 4.11 (br d, J=15.0 Hz, 1H), 3.23-3.10 (m, 2H), 2.92 (br s, 3H), 2.77-2.71 (m, 1H), 2.21-2.11 (m, 1H), 1.87-1.78 (m, 2H), 1.75-1.49 (m, 4H), 1.37 (s, 9H), 0.98 (br d, J=5.5 Hz, 7H).

### Example 994E. 3-((2-Azabicyclo[2.2.1]heptan-5-yl)oxy)-N—((R)-1-(4-amino-7-(3-fluoro-4-(1H-tetrazol-1-yl)phenyl)pyrrolo[2,1-f][1,2,4]triazin-5-yl)piperidin-3-yl)-5-chlorothiophene-2-carboxamide

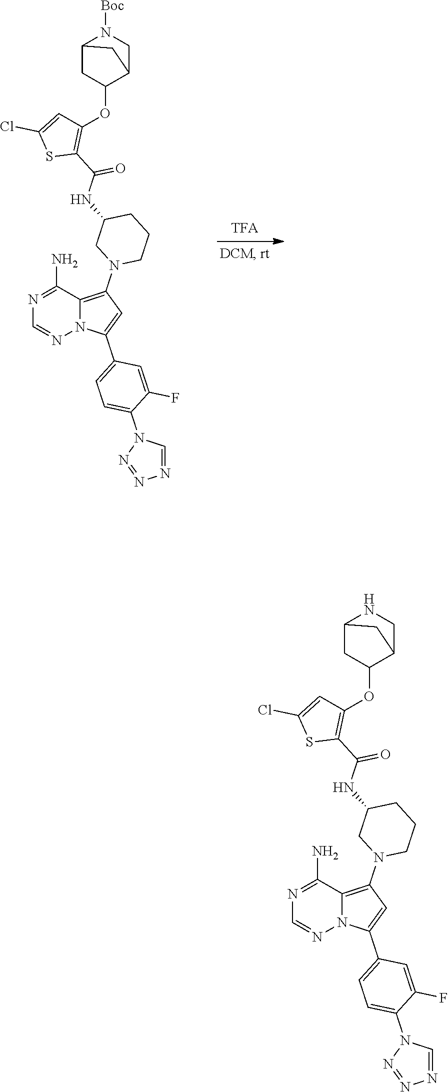 [SMILES: CC(C)(C)OC(=O)N1CC2CC1CC2Oc1cc(Cl)sc1C(=O)N[C@@H]1CCCN(c2cc(-c3ccc(-n4cnnn4)c(F)c3)n3ncnc(N)c23)C1 ; Nc1ncnn2c(-c3ccc(-n4cnnn4)c(F)c3)cc(N3CCC[C@@H](NC(=O)c4sc(Cl)cc4OC4CC5CC4CN5)C3)c12]

Analogous to the preparation of Example 977F, starting with Example 994D (340 mg, 0.453 mmol) and TFA (0.524 mL, 6.80 mmol) in DCM (5 mL) afforded Example 994E (250 mg, 84%) as a brown solid. LC-MS Method E: RT=1.55 min, [M+H]⁺=650.2; LC-MS Method F: RT=1.16 min, [M+H]⁺=650.2; ¹H NMR (400 MHz, DMSO-d₆) δ ppm 9.98 (d, J=1.5 Hz, 1H), 8.61 (br s, 1H), 8.44 (dd, J=1.7, 12.8 Hz, 1H), 8.36-8.12 (m, 3H), 7.23 (br s, 1H), 4.75 (br d, J=5.8 Hz, 1H), 4.17 (br s, 1H), 4.05 (br s, 1H), 3.10 (br d, J=8.0 Hz, 2H), 3.02 (br s, 2H), 2.96-2.80 (m, 4H), 2.60-2.53 (m, 2H), 2.38-2.28 (m, 2H), 1.95-1.74 (m, 5H), 1.63 (br d, J=10.8 Hz, 2H).

### Example 994

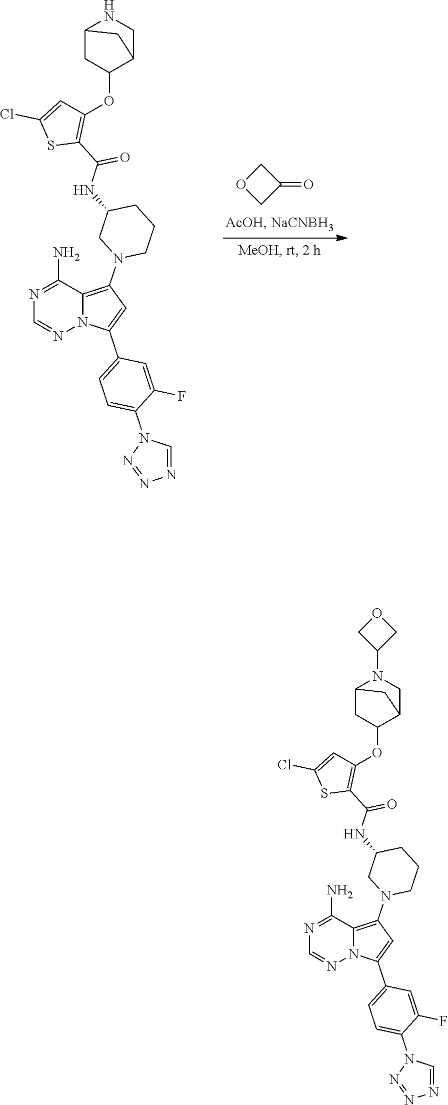 [SMILES: Nc1ncnn2c(-c3ccc(-n4cnnn4)c(F)c3)cc(N3CCC[C@@H](NC(=O)c4sc(Cl)cc4OC4CC5CC4CN5)C3)c12 ; O=C1COC1 ; Nc1ncnn2c(-c3ccc(-n4cnnn4)c(F)c3)cc(N3CCC[C@@H](NC(=O)c4sc(Cl)cc4OC4CC5CC4CN5C4COC4)C3)c12]

Analogous to the preparation of Example 977, starting with Example 994E (40 mg, 0.052 mmol), oxetan-3-one (18.9 mg, 0.262 mmol), acetic acid (4.5 μl, 0.079 mmol) and sodium cyanoborohydride (6.6 mg, 0.105 mmol) in methanol (3 mL) afforded Example 994 (9.5 mg, 25%) as pale-yellow solid. LC-MS Method E: RT=1.76 min, [M+H]⁺=706.2; LC-MS Method F: RT=1.16 min, [M+H]⁺=706.2; ¹H NMR (400 MHz, DMSO-d₆) δ ppm 10.00-9.94 (m, 1H), 8.47-8.40 (m, 1H), 8.33-8.19 (m, 2H), 8.00-7.93 (m, 2H), 7.45-7.38 (m, 1H), 7.28-7.15 (m, 2H), 4.81-4.68 (m, 3H), 4.67-4.59 (m, 1H), 4.57-4.38 (m, 2H), 4.30-3.93 (m, 2H), 3.24-2.76 (m, 8H), 2.15-1.73 (m, 7H), 1.66-1.52 (m, 1H).

### Example 995

### Preparation of N—((R)-1-(4-amino-7-(3-fluoro-4-(1H-tetrazol-1-yl)phenyl)pyrrolo[2,1-f][1,2,4]triazin-5-yl)piperidin-3-yl)-5-chloro-3-((2-(oxetan-3-yl)-2-azabicyclo[2.2.1]heptan-5-yl)oxy)thiophene-2-carboxamide

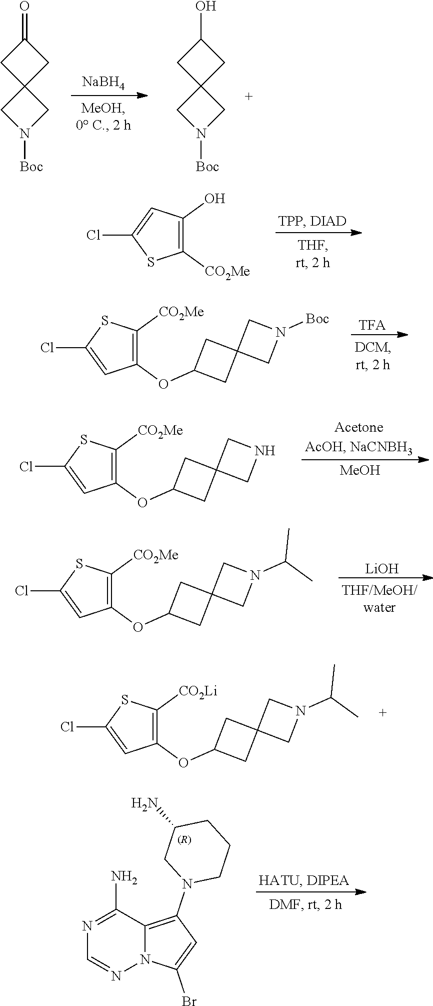

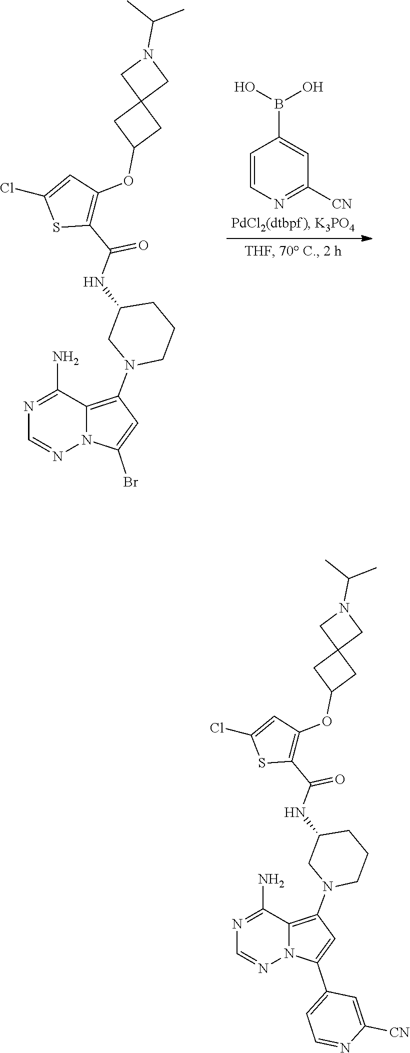 [SMILES: CC(C)N1CC2(CC(Oc3cc(Cl)sc3C(=O)N[C@@H]3CCCN(c4cc(Br)n5ncnc(N)c45)C3)C2)C1 ; N#Cc1cc(B(O)O)ccn1 ; CC(C)N1CC2(CC(Oc3cc(Cl)sc3C(=O)N[C@@H]3CCCN(c4cc(-c5ccnc(C#N)c5)n5ncnc(N)c45)C3)C2)C1]

### Example 995A. tert-Butyl 6-hydroxy-2-azaspiro[3.3]heptane-2-carboxylate

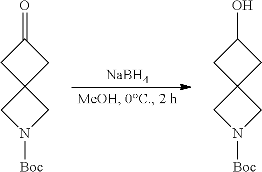 [SMILES: CC(C)(C)OC(=O)N1CC2(CC(=O)C2)C1 ; "NaBH4" ; CC(C)(C)OC(=O)N1CC2(CC(O)C2)C1]

Analogous to the preparation of Example 946A, starting with tert-butyl 6-oxo-2-azaspiro[3.3]heptane-2-carboxylate (5.0 g, 23.7 mmol) and NaBH₄ (1.79 g, 47.3 mmol) in Methanol (50 ml) afforded Example 995A (4.4 g, 87%) as a white solid; ¹H NMR (400 MHz, DMSO-d₆) δ ppm 5.01 (d, J=6.0 Hz, 1H), 3.92 (qd, J=7.0, 14.1 Hz, 1H), 3.79 (br s, 2H), 3.73 (br s, 2H), 2.41-2.35 (m, 2H), 1.95-1.88 (m, 2H), 1.36 (s, 9H).

### Example 995B. tert-Butyl 6-((5-chloro-2-(methoxycarbonyl)thiophen-3-yl)oxy)-2-azaspiro[3.3]heptane-2-carboxylate

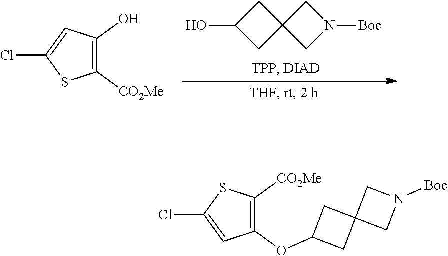 [SMILES: COC(=O)c1sc(Cl)cc1O ; CC(C)(C)OC(=O)N1CC2(CC(O)C2)C1 ; COC(=O)c1sc(Cl)cc1OC1CC2(C1)CN(C(=O)OC(C)(C)C)C2]

Analogous to the preparation of Example 946B, starting with methyl 5-chloro-3-hydroxythiophene-2-carboxylate (2.0 g, 10.4 mmol), Example 995A (2.436 g, 11.4 mmol), triphenylphosphine (4.09 g, 15.6 mmol) and DIAD (3.03 mL, 15.6 mmol) in THF (25 mL) afforded Example 995B (3.5 g, 87%) as a colorless thick liquid. MS: [M+2H-Boc]⁺=288.0; ¹H NMR (400 MHz, CHCl₃-d) δ ppm 6.57 (s, 1H), 4.61 (quin, J=6.8 Hz, 1H), 3.96 (d, J=8.5 Hz, 4H), 3.83 (s, 3H), 2.76-2.67 (m, 2H), 2.54-2.41 (m, 2H), 1.46 (s, 9H).

### Example 995C. Methyl 3-((2-azaspiro[3.3]heptan-6-yl)oxy)-5-chlorothiophene-2-carboxylate, TFA

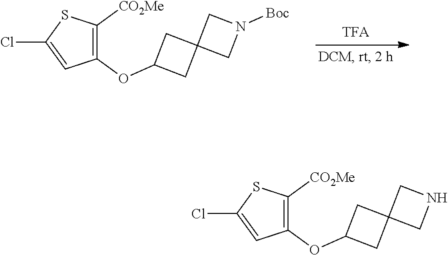 [SMILES: COC(=O)c1sc(Cl)cc1OC1CC2(C1)CN(C(=O)OC(C)(C)C)C2 ; COC(=O)c1sc(Cl)cc1OC1CC2(CNC2)C1]

Analogous to the preparation of Example 946C, starting with Example 995B (1.0 g, 2.58 mmol) and TFA (3.97 mL, 51.6 mmol) in DCM (10 mL) afforded Example 995C (600 mg, 58%) as a white solid. MS: [M+H]⁺=288.2; ¹H NMR (400 MHz, DMSO-d₆) δ ppm 7.17 (s, 1H), 4.70 (quin, J=6.8 Hz, 1H), 3.99 (br d, J=12.0 Hz, 4H), 3.72 (s, 3H), 2.82-2.74 (m, 2H), 2.34-2.26 (m, 2H).

### Example 995D. Methyl 5-chloro-3-((2-isopropyl-2-azaspiro[3.3]heptan-6-yl)oxy)thiophene-2-carboxylate

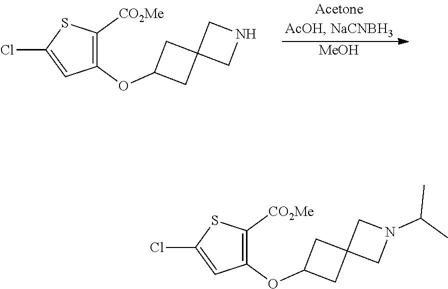 [SMILES: "MeOH" ; COC(=O)c1sc(Cl)cc1OC1CC2(CNC2)C1 ; COC(=O)c1sc(Cl)cc1OC1CC2(C1)CN(C(C)C)C2]

Analogous to the preparation of Example 946D, starting with Example 995C (600 mg, 1.49 mmol), acetone (0.164 mL, 2.24 mmol), acetic acid (0.128 mL, 2.24 mmol) and sodium cyanoborohydride (188 mg, 2.99 mmol) in methanol (5 mL) to afford Example 995D (360 mg, 73%) as an off-white solid. MS: [M+H]⁺=330.2; ¹H NMR (400 MHz, DMSO-d₆) δ ppm 7.17 (s, 1H), 4.77-4.70 (m, 1H), 4.00 (br s, 4H), 3.72 (s, 3H), 2.91-2.66 (m, 2H), 2.39-2.25 (m, 2H), 1.07 (d, J=6.5 Hz, 6H).

### Example 995E. lithium 5-chloro-3-((2-isopropyl-2-azaspiro[3.3]heptan-6-yl)oxy)thiophene-2-carboxylate

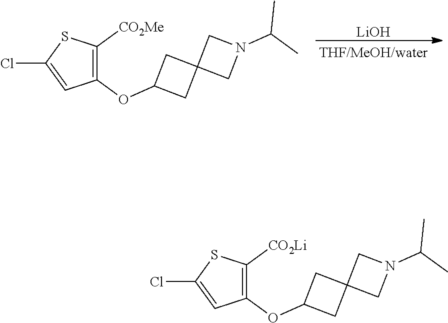

Analogous to the preparation of Example 946E, starting with Example 995D (360 mg, 1.09 mmol) and lithium hydroxide monohydrate (92 mg, 2.18 mmol) in THF/methanol/water (3 mL) to afford Example 995E (350 mg, 100%) as a yellow solid. MS: [M+H]⁺=316.1; ¹H NMR (400 MHz, DMSO-d₆) δ ppm 6.77 (s, 1H), 4.67 (quin, J=7.0 Hz, 1H), 3.03 (d, J=17.5 Hz, 4H), 2.49-2.42 (m, 2H), 2.18-2.06 (m, 3H), 0.80 (d, J=6.0 Hz, 6H).

### Example 995F. (R)—N-(1-(4-Amino-7-bromopyrrolo[2,1-f][1,2,4]triazin-5-yl)piperidin-3-yl)-5-chloro-3-((2-isopropyl-2-azaspiro[3.3]heptan-6-yl)oxy)thiophene-2-carboxamide

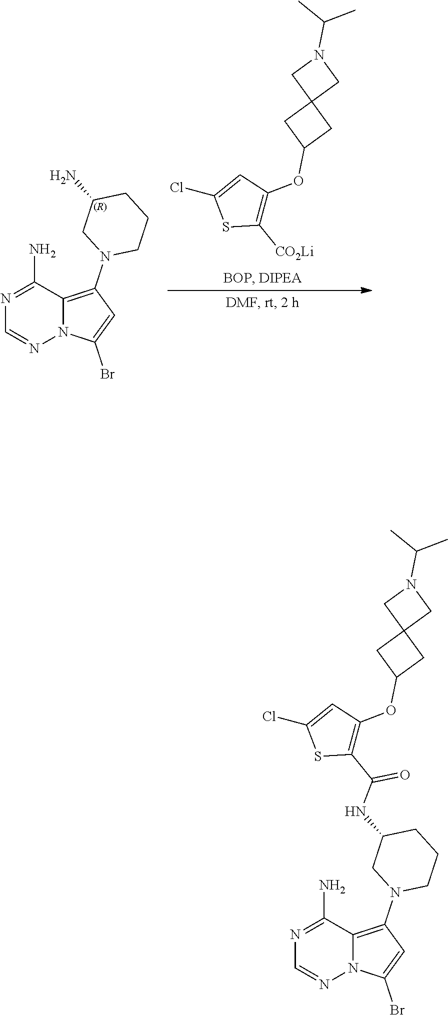

Analogous to the preparation of Example 977C, starting with Intermediate 61, HCl salt (216 mg, 0.622 mmol), Example 995E (200 mg, 0.622 mmol), BOP (275 mg, 0.622 mmol) and DIEA (0.326 mL, 1.86 mmol) in DMF (3 mL) afforded Example 995F (270 mg, 71%) as a yellow solid. MS: [M+H]⁺=608.2; ¹H NMR (400 MHz, DMSO-d₆) δ ppm 8.21-8.00 (m, 1H), 7.83 (s, 1H), 7.15 (s, 1H), 6.79 (s, 1H), 6.71-6.49 (m, 1H), 4.81 (t, J=6.5 Hz, 1H), 4.24-4.09 (m, 1H), 3.02-2.92 (m, 2H), 2.59 (br d, J=6.0 Hz, 2H), 2.44 (br s, 2H), 2.19-2.05 (m, 3H), 0.95 (br d, J=6.0 Hz, 9H), 0.75 (dd, J=2.5, 6.0 Hz, 6H).

### Example 995

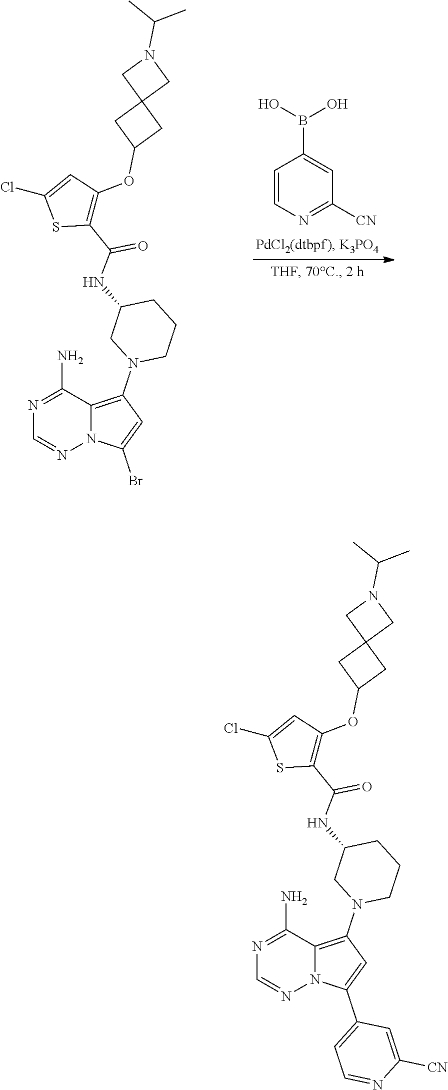 [SMILES: CC(C)N1CC2(CC(Oc3cc(Cl)sc3C(=O)N[C@@H]3CCCN(c4cc(Br)n5ncnc(N)c45)C3)C2)C1 ; N#Cc1cc(B(O)O)ccn1 ; CC(C)N1CC2(CC(Oc3cc(Cl)sc3C(=O)N[C@@H]3CCCN(c4cc(-c5ccnc(C#N)c5)n5ncnc(N)c45)C3)C2)C1]

Analogous to the preparation of Example 970E, starting with Example 995E (70 mg, 0.115 mmol), (2-cyanopyridin-4-yl)boronic acid (17.0 mg, 0.115 mmol), 2M potassium phosphate tribasic (0.115 mL, 0.230 mmol) and [1,1′-bis(di-tert-butylphosphino)ferrocene]dichloropalladium(II) (4.5 mg, 6.9 mmol) in THF (3 mL) afforded Example 995 (13.3 mg, 18%) as a pale-yellow solid. LC-MS Method E: RT=1.46 min, [M+H]⁺=632.2; LC-MS Method F: RT=1.27 min, [M+H]⁺=632.3; ¹H NMR (400 MHz, DMSO-d₆) δ ppm 8.77-8.74 (m, 2H), 8.52 (d, J=5.3 Hz, 1H), 8.31 (br s, 1H), 8.02 (s, 1H), 7.55 (s, 1H), 7.49 (br s, 1H), 7.16 (s, 1H), 4.81 (quin, J=6.5 Hz, 1H), 4.21 (br s, 1H), 3.08-3.04 (m, 2H), 3.01-2.86 (m, 5H), 2.64-2.55 (m, 2H), 2.20-2.07 (m, 3H), 1.91 (s, 1H), 1.84 (br s, 3H), 1.68-1.61 (m, 1H), 1.25-1.12 (m, 1H), 0.73 (br d, J=6.0 Hz, 6H).

### Example 996

### Preparation of N—((R)-1-(4-amino-7-(5-oxo-2,5-dihydrofuran-3-yl) pyrrolo[2,1-f][1,2,4] triazin-5-yl) piperidin-3-yl)-4-((4-(((R)-1-methoxypropan-2-yl) amino)cyclohexyl)oxy)-2-methylthiazole-5-carboxamide

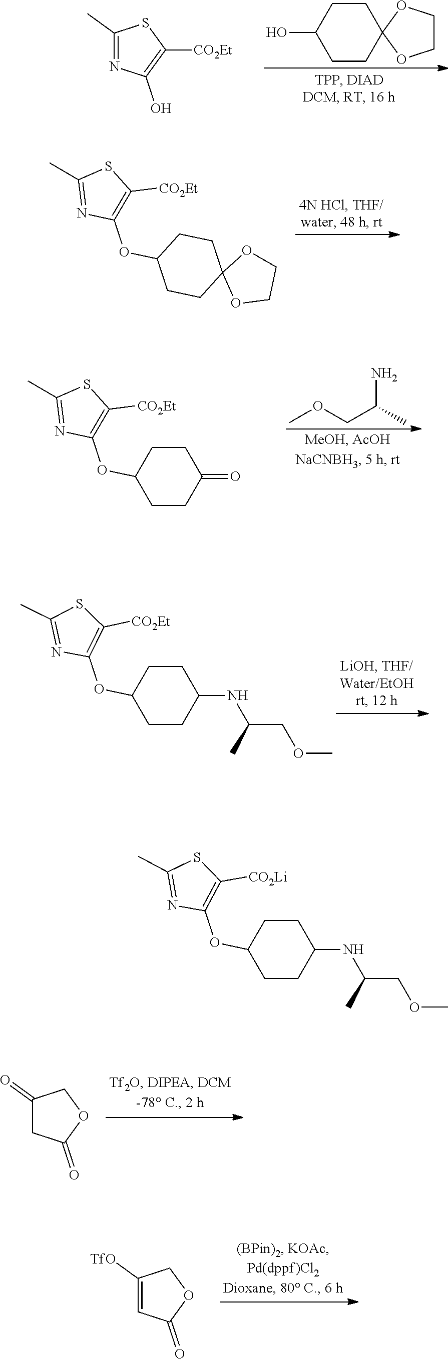

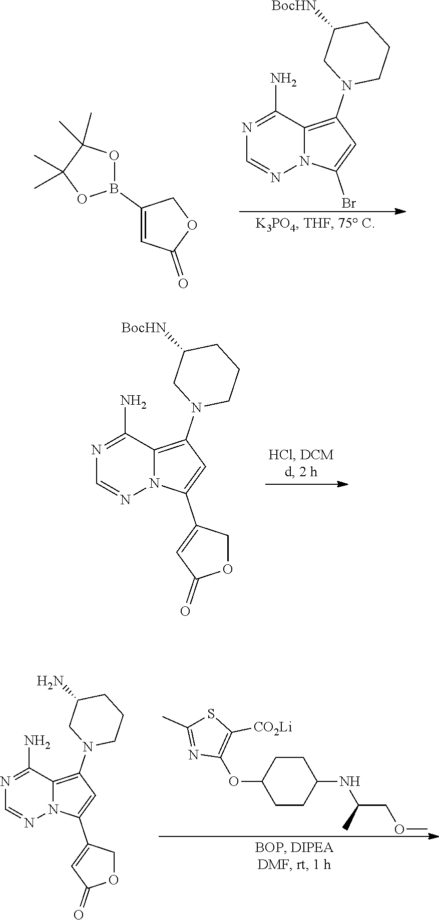

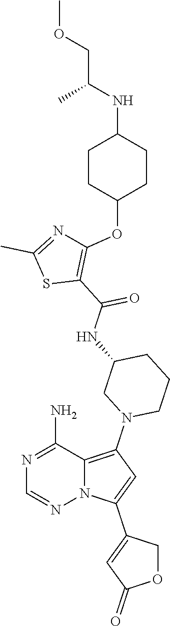 [SMILES: COC[C@@H](C)NC1CCC(Oc2nc(C)sc2C(=O)N[C@@H]2CCCN(c3cc(C4=CC(=O)OC4)n4ncnc(N)c34)C2)CC1]

### Example 996A. Preparation of ethyl 4-((1,4-dioxaspiro [4.5]decan-8-yl) oxy)-2-methylthiazole-5-carboxylate

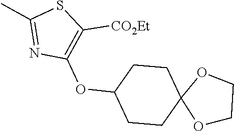 [SMILES: CCOC(=O)c1sc(C)nc1OC1CCC2(CC1)OCCO2]

To a suspension of ethyl 4-hydroxy-2-methylthiazole-5-carboxylate (5.0 g, 26.7 mmol), 1,4-dioxaspiro[4.5]decan-8-ol (5.1 g, 32.0 mmol) and triphenylphosphine (10.5 g, 40.1 mmol) in THF (50 mL) was added DIAD (9.3 mL, 48.1 mmol) dropwise and the mixture was stirred at rt for 16 h. Reaction was diluted with water followed by extraction with ethyl acetate (30 mL×2). The combined ethyl acetate layer was washed with brine (30 mL), dried over anhydrous Na₂SO₄, filtered and concentrated in vacuo to afford yellow gummy solid, which was purified by silica gel chromatography (120 g Redisep® column, eluting with 0-60% EtOAc in Hexane, compound eluted at 18-22%) to afford Example 996A (6.8 g, 71% yield) as yellow gummy solid. MS. [M+H]⁺=328.0; ¹H NMR (300 MHz, CHLOROFORM-d) δ=5.13 (br s, 1H), 4.28 (q, J=6.9 Hz, 2H), 3.96 (br s, 4H), 2.61 (s, 3H), 2.10-1.86 (m, 6H), 1.47-1.21 (m, 5H).

### Example 941B. Preparation of ethyl 2-methyl-4-((4-oxocyclohexyl) oxy) thiazole-5-carboxylate

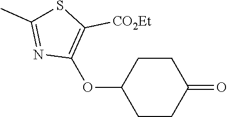 [SMILES: CCOC(=O)c1sc(C)nc1OC1CCC(=O)CC1]

To a stirred solution of the Example 996A (48 g, 127 mmol) in THF (50 mL) and water (50 mL) was added 4N HCl in 1, 4-dioxane (100 mL, 400 mmol). The mixture was stirred at rt for 48 h. Reaction mixture was diluted with THF/EtOAc (1:1, 20 mL) and washed with water (20 mL) and brine solution (20 mL). The organic portion was dried over anhydrous Na₂SO₄, filtered, and concentrated to yield a gummy mass. To this mass diethyl ether (20 mL) was added and the mixture was sonicated, and further concentrated under vacuum to get the white solid. This process again repeated to obtain Example 941B (42 g, 94% yield) as white solid. MS. [M+H]⁺=284.1; ¹H NMR (300 MHz, CHLOROFORM-d) δ=5.39 (br s, 1H), 4.28 (q, J=7.2 Hz, 2H), 2.88-2.75 (m, 2H), 2.63 (s, 3H), 2.42-2.29 (m, 4H), 2.14-1.95 (m, 2H), 1.36-1.30 (m, 3H).

### Example 996B. Preparation of ethyl (R)-4-((4-((1-methoxypropan-2-yl) amino) cyclohexyl) oxy)-2-methylthiazole-5-carboxylate

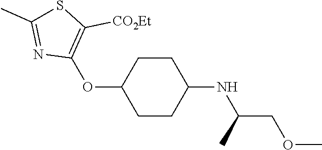

To a stirred solution of Example 941B (800 mg, 2.82 mmol) in MeOH (8 mL) was added (R)-1-methoxypropan-2-amine (579 mg, 6.49 mmol), acetic acid (0.08 mL, 1.41 mmol) and sodium cyanoborohydride (355 mg, 5.65 mmol), and the mixture was allowed to stir for 5 h at it. Reaction was concentrated under reduced pressure. The residue was quenched with saturated solution of sodium bicarbonate (20 mL) followed by extraction with THF/EtOAc (1:1). The combined organic layers were washed with brine (30 mL), dried over anhydrous Na₂SO₄, filtered and concentrated to afford yellow thick mass, which was purified by SFC (Column Name: Lux Cellulose C4 (250*4.6) mm 5μ: % CO₂: 70%: % Co solvent: 30% (5 mM Ammonium acetate in Cameo (1:1); Total Flow: 4 mL/min; Back Pressure: 100bars: Temperature: 35° C.; Detection: UV at 240 nm) to afford first eluting isomer (Iso-1): (350 mg, 35% yield). MS. [M+H]⁺=357.15. 100% ee; ¹H NMR (400 MHz, DMSO-d₆) δ=4.88-4.73 (m, 1H), 4.22-4.12 (m, 4H), 3.26-3.24 (m, 3H), 3.23-3.15 (m, 2H), 2.98-2.90 (m, 1H), 2.69-2.64 (m, 1H), 2.63-2.60 (m, 3H), 2.09-2.00 (m, 2H), 1.54-1.39 (m, 2H), 1.27-1.20 (m, 4H), 1.18-1.09 (m, 1H), 1.00-0.94 (m, 3H) and second eluting isomer (Example 996B, Iso-2): (600 mg, 59% yield). MS. [M+H]⁺=357.15. 97.91% ee; ¹H NMR (400 MHz, DMSO-d₆) δ=5.16-5.03 (m, 1H), 4.19 (d, J=7.1 Hz, 4H), 3.26 (s, 3H), 3.24-3.21 (m, 2H), 3.06-2.99 (m, 1H), 2.75-2.66 (m, 1H), 2.61 (s, 3H), 1.76-1.38 (m, 6H), 1.27 (s, 3H), 0.98 (d, J=6.4 Hz, 3H). The second eluting isomer (Example 996B, Iso-2) was taken to next step.

### Example 996C. Preparation of lithium (R)-4-((4-((1-methoxypropan-2-yl) amino) cyclohexyl) oxy)-2-methylthiazole-5-carboxylate

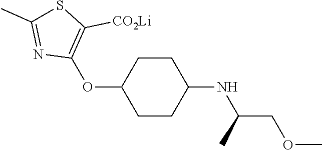

To a stirred solution of Example 996B (600 mg, 1.68 mmol) in ethanol (1 mL)/THF (1 mL)/water (0.5 mL) was added LiOH·H₂O (121 mg, 5.05 mmol) and reaction was stirred at rt for 12 h. The reaction mixture was concentrated, azeotroped with toluene to afford Example 996C (450 mg, 65% yield) as white solid. MS. [M+H]⁺=329.3.

### Example 996D. Preparation of 5-oxo-2,5-dihydrofuran-3-yl trifluoromethanesulfonate

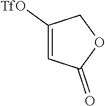 [SMILES: O=C1C=C(OS(=O)(=O)C(F)(F)F)CO1]

To a mixture of 4-hydroxyfuran-2(5H)-one (5.0 g, 50.0 mmol) in DCM (50 mL) at −78° C. was added DIPEA (8.73 mL, 50.0 mmol) followed by addition of trifluoromethanesulfonic anhydride (8.44 mL, 50.0 mmol) drop-wise and the mixture stirred at the same temperature for 2 h. The reaction mixture was diluted with DCM (50 mL) and then quenched with cold water (100 mL). The organic layer was separated, washed with brine (1×30 mL), dried over anhydrous sodium sulfate and concentrated in vacuo to afford the crude material which was purified by silica gel chromatography (40 g Redisep® column, 0-50% EtOAc in hexane to afford the Example 996D (8 g, 69% yield) as yellow liquid; ¹H NMR (300 MHz, CHLOROFORM-d) δ=6.15-5.96 (m, 1H), 4.91 (d, J=1.9 Hz, 2H). ¹⁹F NMR (300 MHz, CHLOROFORM-d) δ=−72.48.

### Example 996E. Preparation of 2-(2,2-difluoroethoxy)-5-(4,4,5,5-tetramethyl-1,3,2-dioxaborolan-2-yl) pyridine

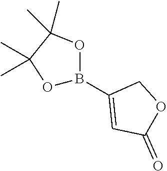

The mixture of Example 996D (9.5 g, 40.9 mmol), bispin (11.43 g, 45.0 mmol) and potassium acetate (4.8 g, 49.1 mmol) in dioxane (150 mL) was degassed by purging argon for 10 min. and then PdCl₂(dppf)·DCM complex (2.0 g, 2.45 mmol) was added. The mixture was stirred at 80° C. for 6 h. Reaction temperature was allowed to come to rt and then diluted with ethyl acetate (100 mL) and filtered through celite. The organic portion was washed with water (3×25 mL) followed by brine (1×30 mL), dried over anhydrous sodium sulfate and concentrated in vacuo to afford Example 996E (8.6 g, 100% yield) as brown gummy solid; ¹H NMR (400 MHz, CHLOROFORM-d) δ=6.59-6.49 (m, 1H), 4.99-4.94 (m, 2H), 1.35-1.31 (m, 12H).

### Example 996F. Preparation of tert-butyl (R)-(1-(4-amino-7-(5-oxo-2,5-dihydrofuran-3-yl) pyrrole[2,1-f][1,2,4]triazin-5-yl)piperidin-3-yl)carbamate

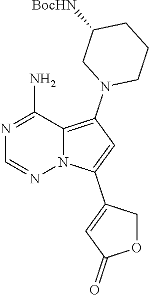 [SMILES: CC(C)(C)OC(=O)N[C@@H]1CCCN(c2cc(C3=CC(=O)OC3)n3ncnc(N)c23)C1]

To a stirred solution of tert-butyl (R)-(1-(4-amino-7-bromopyrrolo[2,1-f][1,2,4]triazin-5-yl)piperidin-3-yl)carbamate (5.5 g, 13.3 mmol) and Example 996E (16.8 g, 40.1 mmol) in THF (60 mL) was added aqueous potassium phosphate solution (2M, 10.0 mL, 20.0 mmol). The mixture was purged with argon for 5 min. and subsequently PdCl₂(dppf)·DCM complex (1.1 g, 1.34 mmol) was added, and then the reaction mixture was stirred at 70° C. for 3 h. The reaction was cooled to rt and then diluted with ethyl acetate/THF (1:1, 100 mL). The organic portion was washed with water (50 mL 2) and brine solution (50 mL) solution. The organic portion was dried over anhydrous Na₂SO₄, filtered, and concentrated to afford brown solid, which was purified by silica gel chromatography (120 g Redisep® column, 0-100% EtOAc in DCM, compound eluted at 70-90%) to afford the Example 996F (4.6 g, 82% yield) as yellow solid. MS. [M+H]⁺=415.20; ¹H NMR (400 MHz, DMSO-d₆) δ=8.32-8.14 (m, 1H), 8.05-7.94 (m, 1H), 7.20-6.95 (m, 3H), 6.70-6.58 (m, 1H), 5.47-5.29 (m, 2H), 3.92 (s, 2H), 3.76-3.57 (m, 1H), 3.09-2.61 (m, 3H), 1.89-1.57 (m, 3H), 1.44-1.27 (m, 9H).

### Example 996G. Preparation of (R)-4-(4-amino-5-(3-aminopiperidin-1-yl) pyrrole[2,1-f][1,2,4]triazin-7-yl) furan-2(5H)-one. HCl

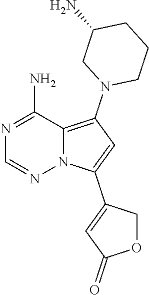 [SMILES: Nc1ncnn2c(C3=CC(=O)OC3)cc(N3CCC[C@@H](N)C3)c12]

To a stirred solution of Example 996F (4.6 g, 11.1 mmol) in DCM (40 mL) was added 4N HCl in dioxane (27.7 mL, 111 mmol) at 0° C. and stirred at rt for 2 h. Reaction was concentrated in vacuo and the residue was triturated with diethyl ether (3×10 mL). The solid was filtered, washed with diethyl ether (20 mL) and dried to afford Example 996G (3.2 g, 73% yield) as an off-white solid. MS. [M+H]⁺=286.9; ¹H NMR (400 MHz, DMSO-d₆) δ=8.47 (br s, 2H), 8.12 (s, 1H), 7.60 (br s, 1H), 7.20-7.12 (m, 2H), 6.76-6.72 (m, 1H), 5.45-5.38 (m, 2H), 3.18 (br d, J=9.5 Hz, 1H), 3.01 (br s, 2H), 2.68 (br s, 2H), 2.16-1.96 (m, 1H), 1.85 (br s, 1H), 1.71 (br s, 2H).

### Example 996

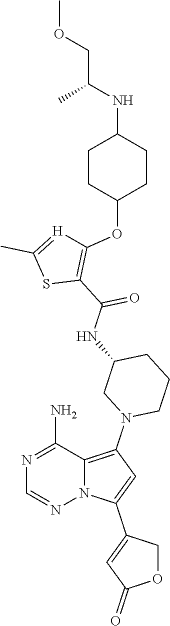

To an ice cold mixture of Example 996G (210 mg, 0.59 mmol), Example 996C (200 mg, 0.59 mmol) and BOP (317 mg, 0.72 mmol) in DMF (3 mL) was added DIPEA (0.31 mL, 1.79 mmol) and the mixture was stirred at rt for 1 h. The reaction mixture was quenched by adding cold water (5 mL) and diluted with ethyl acetate (10 mL). The organic layer was separated and washed with water (3×5 mL) and brine (1-5 mL). The organic portion concentrated in vacuo to get the crude which was purified by reverse phase purification (Column: Gemini NX C18 (19 mm×250 mm*5μ); Mobile phase A: 10 mm Ammonium Bicarbonate in Water pH-9.5; Mobile phase B: ACN: Flow:20 ml/min; Gradient Condition:0/30, 20/80, 21/100) to afford Example 996 (95 mg, 25% yield) as an off white solid. MS. [M+H]⁺=625.35; HPLC Method G: RT=6.42 min, Method H: RT=8.32 min; ¹H NMR (400 MHz, DMSO-d₆) δ=8.33 (br s, 1H), 7.99 (s, 1H), 7.27 (br s, 1H), 7.13 (s, 1H), 6.82 (br s, 1H), 6.64 (s, 1H), 5.39 (s, 2H), 5.15 (br s, 1H), 4.18 (br s, 1H), 3.25-3.16 (m, 3H), 3.15-3.01 (m, 2H), 2.98 (br s, 1H), 2.81 (br s, 2H), 2.67 (br s, 1H), 2.63-2.52 (m, 6H), 1.92 (br s, 3H), 1.84 (br s, 2H), 1.65 (br s, 4H), 1.39-1.12 (m, 3H), 0.87 (br d, J=6.1 Hz, 3H).

The following examples in Table 68 were prepared using the same procedure as described for Example 1. Lithium salt of the acids were prepared using the procedures described for Example 996A-996C.

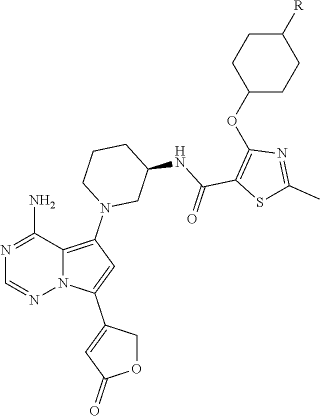

TABLE 1

| | | | | HPLC | |
|---|---|---|---|---|---|
| | | | | Method, | |
| | Structure | | LCMS | RT | |
| Example | (R) | Name | (M+H)+ | (min.) | HNMR |
| 997 | -OH | (R)-N-(1-(4-amino-7-(5-oxo- | 609.3 | A: 1.26 | (400 MHZ, DMSO-d6) 8 ppm 8.44 (s, 1H), 8.01 (s, |
| | | 2,5-dihydrofuran-3-y1) | | B: 0.96 | 1H), 7.30 (br s, 1H), 7.14 (s, IH), 6.79 (s, IH), 6.65 |
| | | pyrrolo[2,1-f] [1,2,4]triazin-5- | | | (t, J = 1.6 Hz, IH), 5.40 (d, J = 1.5 Hz, 2H), 5.28 (s, |
| | | y1)piperidin-3-y1)-4-((4~(3- | | | IH), 5.06 (br s, IH), 4.18 (br s, IH), 4.09 (br s, 1H), |
| | | hydroxyazetidin-1- | | | 3.47 (br s, 3H), 3.18 (s, 2H), 2.92 (br s, 2H), 2.60 (s, |
| | | yl)cyclohexyl)oxy)-2- | | | 3H), 1.98 - 1.81 (m, 6H), 1.71 - 1.40 (m, 4H), 1.37 |
| | | methylthiazole-5-carboxamide | | | - 1.26 (m, 2H). |
| Example | Structure | Name | LCMS | HPLC | IHNMR |
| | (R) | | (M+H) | Method, | |
| | | | + | RT | |
| | | | | (min.) | |
| 998 | N. | (R)-N-(1-(4-amino-7-(5-oxo- | 593.3 | A: 1.27 | (400 MHz, DMSO-d6) 8 = 8.50 (br s, 1H), 8.00 (s, |
| | | 2,5-dihydrofuran-3-y1) | | B: 0.98 | IH), 7.33 (br s, IH), 7.16 (s, IH), 6.80 (br s, 1H), |
| | | pyrrolo[2,1-f] [1,2,4]triazin-5- | | | 6.64 (s, IH), 5.42 - 5.36 (m, 2H), 5.04 (br s, IH), |
| | | yl)piperidin-3-y1)-4-((4- | | | 4.21 (br s, IH), 3.23 - 3.12 (m, IH), 3.08 - 2.85 (m, |
| | | (azetidin-1- | | | 6H), 2.62 - 2.52 (m, 5H), 2.05 (br s, 1H), 1.93 - 1.79 |
| | | yl)cyclohexyl)oxy)-2- | | | (m, 6H), 1.64 (br d, J = 4.6 Hz, 3H), 1.44 (br s, 2H), |
| | | methylthiazole-5-carboxamide | | | 1.29 (br d, J = 9.5 Hz, 2H) |
| Example | Structure | Name | LCMS | HPLC | IHNMR |
| | (R) | | (M+H) | Method, | |
| | | | + | RT | |
| | | | | (min.) | |
| 999 | | (R)-N-(1-(4-amino-7-(5-oxo- | 607.3 | A: 1.38 | (400 MHZ, DMSO-d6) 8 = 8.43 (br s, IH), 8.01 (s, |
| | | 2,5-dihydrofuran-3-y1) | | B: 1.02 | IH), 7.29 (br s, IH), 7.16 (s, 1H), 6.82 (br s, IH), |
| | | pyrrolo[2,1-f] [1,2,4]triazin-5- | | | 6.64 (t, J = 1.6 Hz, IH), 5.39 (d, J =1.5 Hz, 2H), |
| | | yl)piperidin-3-y1)-2-methyl-4- | | | 5.06 (br dd, J = 1.5, 3.0 Hz, IH), 4.21 (br s, IH), |
| | | ((4-(3-methylazetidin-1- | | | 3.20 (br s, 4H), 2.95 (br s, 2H), 2.60 (s, 4H), 2.33 (S, |
| | | yl)cyclohexyl)oxy)thiazole-5- | | | 1H), 2.08 (s, IH), 1.98 - 1.73 (m, 6H), 1.70 - 1.54 |
| | | carboxamide | | | (m, 3H), 1.49 (br s, 2H), 1.28 (br dd, J = 5.3, 8.5 Hz, |
| | | | | | 2H), 1.02 (d, J = 6.8 Hz, 3H) |

### Example 1000

### Preparation of (R)—N-(1-(4-amino-7-(5-oxo-2,5-dihydrofuran-3-yl) pyrrolo[2,1-f][1,2,4]triazin-5-yl)piperidin-3-yl)-4-((4-((1-methoxy-2-methylpropan-2-yl)amino)cyclohexyl) oxy)-2-methylthiazole-5-carboxamide

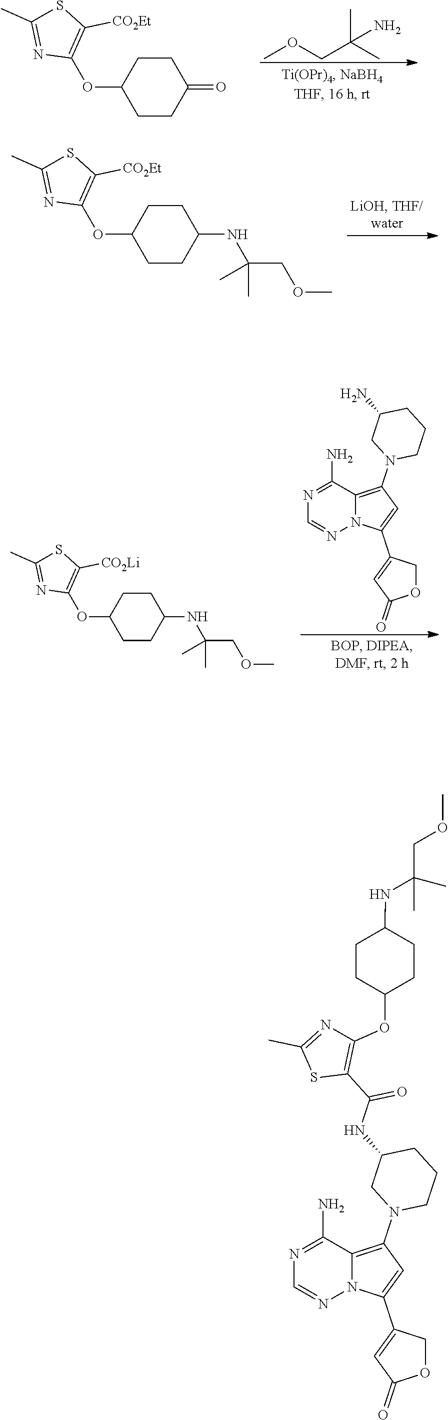

### Example 1000A. Preparation of ethyl 4-((4-((1-methoxy-2-methylpropan-2-yl) amino) cyclohexyl)oxy)-2-methylthiazole-5-carboxylate

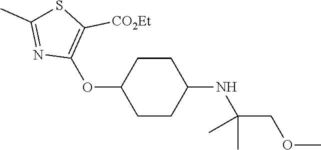

To a stirred solution of Example 941B (1.0 g, 3.53 mmol) in THF (5 mL) was added 1-methoxy-2-methylpropan-2-amine (1.82 g, 17.65 mmol) and stirred at rt for 5 min. and subsequently titanium(IV) isopropoxide (2.09 mL, 7.06 mmol) was added to the reaction mixture drop-wise and the resulting solution was stirred at rt for 14 h. NaBH₄ (0.2 g, 5.29 mmol) was added and further stirred at rt for 2 h. Reaction was diluted with methanol (30 mL)/THF (30 mL) and filtered through celite bed. The filtrate was concentrated and purified by silica gel chromatography (120 g Redisep® column, eluting with 0-20% MeOH in DCM to afford Example 1000A (400 mg, 31% yield) as yellow gummy solid. MS. [M+H]⁺=286.9; ¹H NMR (400 MHz, CHLOROFORM-d) δ=5.15-5.09 (m, 1H), 3.37 (s, 4H), 3.21-3.15 (m, 2H), 2.62 (s, 5H), 2.13-2.02 (m, 2H), 1.93-1.69 (m, 3H), 1.64-1.59 (m, 2H), 1.37 (t, J=7.3 Hz, 4H), 1.10 (s, 6H).

### Example 1000B. Preparation of lithium 4-((4-((1-methoxy-2-methylpropan-2-yl)amino)cyclohexyl)oxy)-2-methylthiazole-5-carboxylate

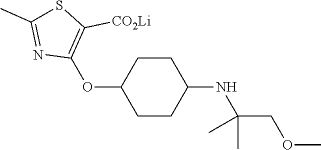

Analogous to the preparation of Example 996C, reaction of Example 1000A (400 mg, 1.08 mmol) and lithium hydroxide hydrate (91 mg, 2.16 mmol) in THF (2.5 mL)/ethanol (2.5 mL)/water (1.5 mL) at rt for 16 h afforded Example 1000B (340 mg, 90% yield) as white solid. MS. [M+H]⁺=343.2

### Example 1000

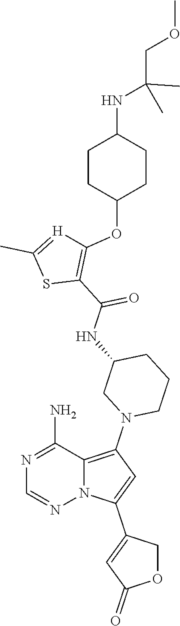

Analogous to the preparation of Example 996, reaction Example 996C (60.4 mg, 0.17 mmol), Example 1000B (60 mg, 0.17 mmol) BOP (91 mg, 0.21 mmol) and DIPEA (0.09 mL, 0.52 mmol) in DMF (3 mL) at rt for 2 h afforded the crude which was purified via reverse phase chromatography to afford Example 5 (28.9 mg, 26% yield) as pale-yellow solid. LC-MS Method E: RT=1.50 min, [M+H]⁺=639.3; LC-MS Method B: RT=1.11 min, [M+H]⁺=639.3; ¹H NMR (400 MHz, DMSO-d₆) δ=8.32 (br s, 1H), 8.12 (br d, J=3.5 Hz, 2H), 8.01 (s, 1H), 7.25 (s, 1H), 7.13 (d, J=4.5 Hz, 3H), 6.99 (s, 1H), 6.94 (br s, 1H), 6.67-6.57 (m, 1H), 5.39 (s, 2H), 5.05 (br s, 1H), 4.14 (br s, 1H), 3.33 (s, 3H), 3.04 (br s, 1H), 2.67 (br s, 2H), 2.61 (s, 3H), 2.17 (br d, J=12.0 Hz, 2H), 2.04 (br s, 1H), 1.93-1.60 (m, 8H), 1.50 (br s, 1H), 1.28 (s, 6H).

### Example 1001

### Preparation of (R)—N-(1-(4-amino-7-(5-oxo-2,5-dihydrofuran-3-yl)pyrrolo[2,1-f][1,2,4]triazin-5-yl)piperidin-3-yl)-2-methyl-4-((4-(tert-pentylamino)cyclohexyl)oxy)thiazole-5-carboxamide

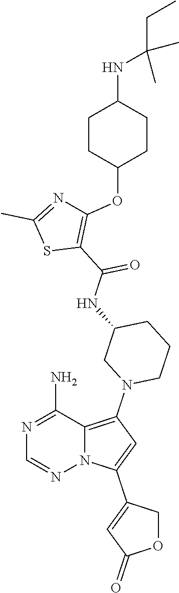 [SMILES: CCC(C)(C)NC1CCC(Oc2nc(C)sc2C(=O)N[C@@H]2CCCN(c3cc(C4=CC(=O)OC4)n4ncnc(N)c34)C2)CC1]

Analogous to the preparation of Example 996, reaction Example 996C (84 mg, 0.24 mmol), lithium 2-methyl-4-((4-(tert-pentylamino)cyclohexyl)oxy)thiazole-5-carboxylate (80 mg, 0.24 mmol), BOP (128 mg, 0.29 mmol) and DIPEA (0.12 mL, 0.72 mmol) in DMF (3 mL) at rt for 2 h afforded the crude which was purified via reverse phase chromatography to afford Example 1001 (54.5 mg, 36% yield) as pale yellow solid. LC-MS Method E: RT=1.48 min, [M+H]⁺=623.3; LC-MS Method F: RT=1.12 min, [M+H]⁺=623.3. ¹H NMR (400 MHz, DMSO-d₆) δ=8.34 (br s, 1H), 8.00 (s, 1H), 7.32 (br s, 1H), 7.15 (s, 1H), 6.80 (br s, 1H), 6.64 (s, 1H), 5.38 (s, 2H), 5.12 (br s, 1H), 4.21 (br s, 1H), 3.23 (br s, 3H), 2.95 (br s, 3H), 2.60 (s, 3H), 2.02-1.77 (m, 5H), 1.74-1.38 (m, 4H), 1.36-1.12 (m, 4H), 0.85 (br s, 6H), 0.76-0.61 (m, 3H).

### Example 1002

### Preparation of N—((R)-1-(4-amino-7-(5-oxo-2,5-dihydrofuran-3-yl)pyrrolo[2,1-f][1,2,4]triazin-5-yl)piperidin-3-yl)-2-methyl-4-(((1S,4S)-4-((tetrahydro-2H-pyran-4-yl)amino)cyclohexyl)oxy)thiazole-5-carboxamide

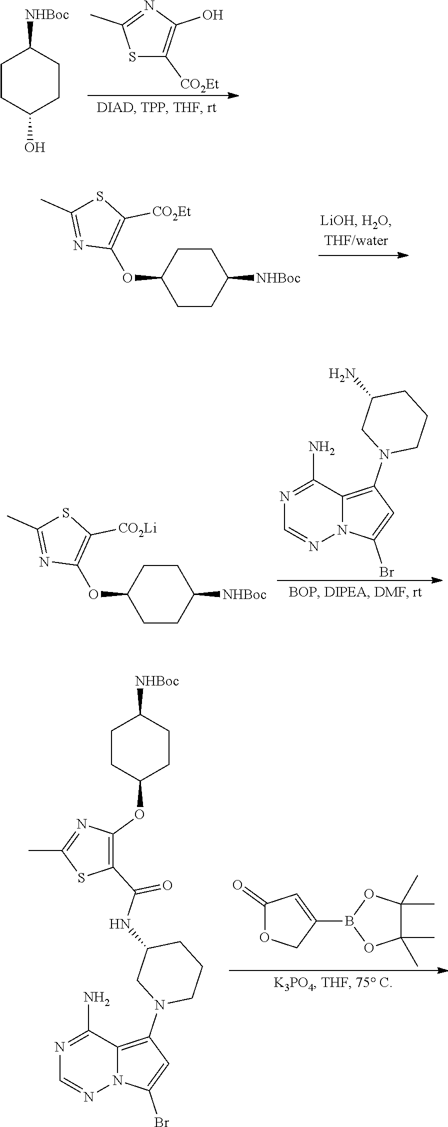

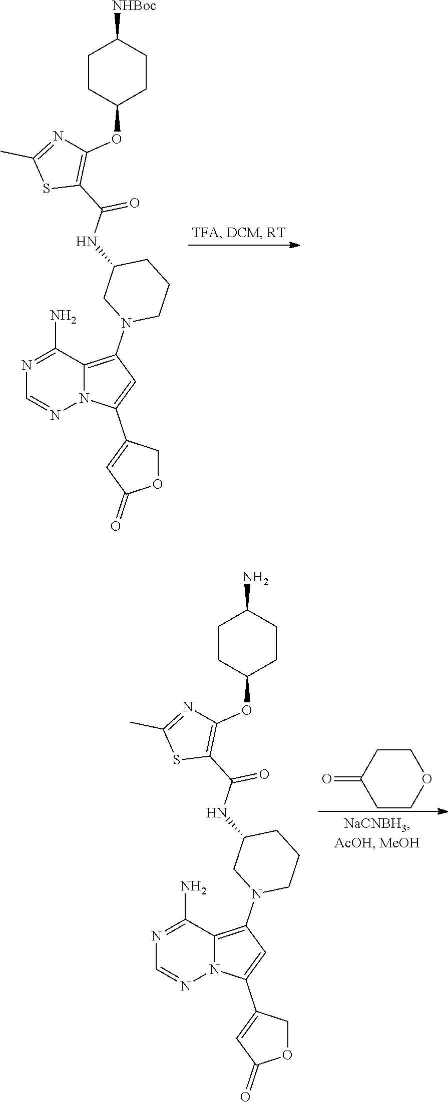 [SMILES: Cc1nc(O[C@H]2CC[C@@H](NC(=O)OC(C)(C)C)CC2)c(C(=O)N[C@@H]2CCCN(c3cc(C4=CC(=O)OC4)n4ncnc(N)c34)C2)s1 ; Cc1nc(O[C@H]2CC[C@@H](N)CC2)c(C(=O)N[C@@H]2CCCN(c3cc(C4=CC(=O)OC4)n4ncnc(N)c34)C2)s1 ; O=C1CCOCC1]

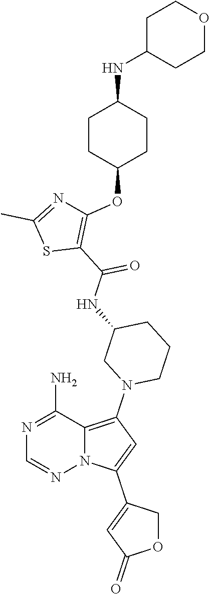 [SMILES: Cc1nc(O[C@H]2CC[C@@H](NC3CCOCC3)CC2)c(C(=O)N[C@@H]2CCCN(c3cc(C4=CC(=O)OC4)n4ncnc(N)c34)C2)s1]

### Example 1002A. Preparation of ethyl 4-(((1S,4S)-4-((tert-butoxy carbonyl) amino) cyclohexyl) oxy)-2-methylthiazole-5-carboxylate

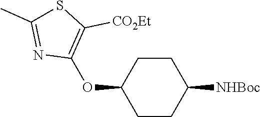 [SMILES: CCOC(=O)c1sc(C)nc1O[C@H]1CC[C@@H](NC(=O)OC(C)(C)C)CC1]

Analogous to the preparation of Example 996A, reaction of ethyl 4-hydroxy-2-methylthiazole-5-carboxylate (10 g, 53.4 mmol), tert-butyl ((1R,4R)-4-hydroxycyclohexyl)carbamate (11.5 g, 53.4 mmol), triphenylphosphine (21.02 g, 80 mmol) and DIAD (23.89 mL, 123 mmol) in THF (100 mL) afforded the crude which was purified by silica gel chromatography (330 g Redisep® column, eluting with 0-100% EA in Pet.Ether, compound eluted at 28-30%) to afford Example 1002A (13.5 g, 42% yield) as white solid. MS: [M+H]⁺=385.15; ¹H NMR (400 MHz, CHLOROFORM-d) δ=5.19-5.14 (m, 1H), 4.32-4.25 (m, 2H), 2.62 (s, 3H), 2.10-1.98 (m, 2H), 1.84-1.78 (m, 2H), 1.72-1.65 (m, 3H), 1.46 (s, 9H), 1.35 (d, J=7.0 Hz, 6H).

### Example 1002B. Preparation of lithium 4-(((1S,4S)-4-((tert-butoxycarbonyl)amino)cyclohexyl)oxy)-2-methylthiazole-5-carboxylate

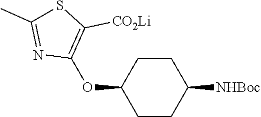

Analogous to the preparation of Example 996C, reaction of Example 1002A (6 g, 15.61 mmol) and LiOH·H₂O (1.12 g, 46.8 mmol) in ethanol (20 mL)/THF (30 mL)/water (10 mL) afforded Example 1002B (6.7 g, 95% yield). MS. [M−H]⁺=355.05. ¹H NMR (400 MHz, CHLOROFORM-d) δ=5.03-4.94 (m, 1H), 3.95-3.73 (m, 1H), 2.60-2.51 (m, 3H), 2.64-2.42 (m, 3H), 2.09-1.83 (m, 6H), 1.42 (s, 9H).

### Example 1002C. Preparation of tert-butyl ((1S,4S)-4-((5-(((R)-1-(4-amino-7-bromopyrrolo[2,1-f][1,2,4]triazin-5-yl)piperidin-3-yl)carbamoyl)-2-methylthiazol-4-yl)oxy)cyclohexyl)carbamate

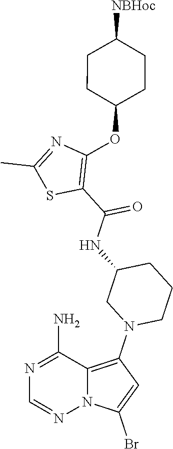

Analogous to the preparation of Example 996, reaction of (R)-5-(3-aminopiperidin-1-yl)-7-bromopyrrolo[2,1-f][1,2,4]triazin-4-amine. HCl (4 g, 11.51 mmol), Example 1002B (4.17 g, 11.51 mmol), BOP (6.11 g, 13.81 mmol), DIPEA (10.0 mL, 57.5 mmol) in DMF (30 mL) afforded crude product which was purified by silica gel chromatography (330 g Redisep® column, eluting with 0-20% MeOH in DCM) to afford the Example 1002C (14.37 g, 82% yield) as off-white solid. MS. [M−H]⁺=651.20. ¹H NMR (400 MHz, DMSO-d₆) δ=7.83 (s, 1H), 7.22-7.06 (m, 1H), 6.86-6.66 (m, 2H), 5.13-4.96 (m, 1H), 4.19-4.02 (m, 1H), 3.67-3.56 (m, 1H), 3.27-3.21 (m, 2H), 3.22-3.02 (m, 3H), 3.03-2.89 (m, 1H), 2.60 (s, 3H), 2.05-1.94 (m, 3H), 1.88-1.78 (m, 2H), 1.77-1.63 (m, 4H), 1.56-1.45 (m, 3H), 1.40-1.36 (m, 9H).

### Example 1002D. Preparation of tert-butyl ((1S,4S)-4-((5-(((R)-1-(4-amino-7-(5-oxo-2,5-dihydrofuran-3-yl)pyrrolo[2,1-f][1,2,4]triazin-5-yl)piperidin-3-yl)carbamoyl)-2-methylthiazol-4-yl)oxy)cyclohexyl)carbamate

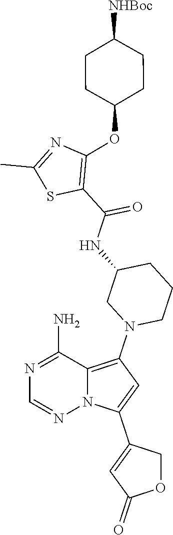 [SMILES: Cc1nc(O[C@H]2CC[C@@H](NC(=O)OC(C)(C)C)CC2)c(C(=O)N[C@@H]2CCCN(c3cc(C4=CC(=O)OC4)n4ncnc(N)c34)C2)s1]

Analogous to the preparation of Example 9% F, reaction of Example 1002C (950 mg, 1.46 mmol), Example 996E (1843 mg, 4.39 mmol), aqueous potassium phosphate solution (2M, 1.09 mL, 2.19 mmol) and Pd(dppf)Cl₂·DCM complex (119 mg, 0.14 mmol) in THF (15 mL) afforded crude product which was purified by silica gel chromatography (40 g Redisep® column, eluting with 0-20% MeOH in DCM) to afford the Example 1002D (550 mg, 39% yield) as yellow solid. MS. [M−H]⁺=651.20. ¹H NMR (400 MHz, DMSO-d₆) δ=7.96 (s, 1H), 7.09 (s, 1H), 6.63 (s, 1H), 5.36 (s, 2H), 5.02 (br s, 1H), 4.14-4.00 (m, 2H), 3.31-3.16 (m, 1H), 3.00-2.91 (m, 1H), 2.59-2.53 (m, 4H), 1.95 (br s, 3H), 1.81 (br s, 2H), 1.67 (br s, 4H), 1.45 (br d, J=17.5 Hz, 3H), 1.37-1.25 (m, 10H).

### Example 1002E. Preparation of N—((R)-1-(4-amino-7-(5-oxo-2,5-dihydrofuran-3-yl)pyrrolo[2,1-f][1,2,4]triazin-5-yl)piperidin-3-yl)-4-(((1S,4S)-4-aminocyclohexyl)oxy)-2-methylthiazole-5-carboxamide. TFA

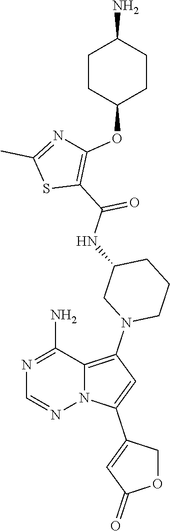 [SMILES: Cc1nc(O[C@H]2CC[C@@H](N)CC2)c(C(=O)N[C@@H]2CCCN(c3cc(C4=CC(=O)OC4)n4ncnc(N)c34)C2)s1]

To a solution of Example 1002D (550 mg, 0.84 mmol) in DCM (5 mL) was added TFA (0.97 mL, 12.64 mmol) and stirred at rt for 2 h. The reaction mixture was concentrated, followed by the addition of diethyl ether (30 mL). After trituration, the ether layer was decanted, and the sediment was further concentrated to yield Example 1002E (450 mg, 80% yield) as yellow solid. MS. [M−H]⁺=651.20; ¹H NMR (400 MHz, DMSO-d₆) δ=8.50-8.31 (m, 1H), 8.06-7.99 (m, 1H), 7.89-7.79 (m, 2H), 7.17-7.08 (m, 2H), 7.04-6.93 (m, 1H), 6.70-6.62 (m, 1H), 5.45-5.34 (m, 2H), 5.06-4.96 (m, 1H), 3.32-2.91 (m, 6H), 2.60 (s, 3H), 2.21-2.09 (m, 2H), 1.94-1.67 (m, 6H), 1.63-1.45 (m, 4H).

### Example 1002

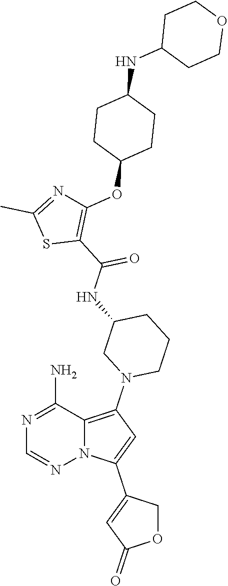 [SMILES: Cc1nc(O[C@H]2CC[C@@H](NC3CCOCC3)CC2)c(C(=O)N[C@@H]2CCCN(c3cc(C4=CC(=O)OC4)n4ncnc(N)c34)C2)s1]

Analogous to the preparation of Example 996B, reaction of Example 1002E (40 mg, 0.06 mmol), acetic acid (3.43 μl, 0.06 mmol), sodium cyanoborohydride (7.54 mg, 0.12 mmol) and tetrahydro-4H-pyran-4-one (18.02 mg, 0.18 mmol) in methanol (1.5 mL) afforded crude material which was purified via preparative LC/MS with the following conditions: Column: Waters XBridge C18, 150 mm×19 mm, 5-μm particles; Mobile Phase A: 5:95 acetonitrile: water with 10-mM ammonium acetate; Mobile Phase B: 95:5 acetonitrile:water with 10-mM ammonium acetate; Gradient: 12-32% B over 25 minutes, then a 5-minute hold at 100% B: Flow Rate: 20 mL/min; Column Temperature: 25 C. Fraction collection was triggered by signals. Fractions containing the desired product were combined and dried via centrifugal evaporation to afford Example 1002 (13.9 mg, 36% yield) as pale-yellow solid. LC-MS Method E: RT=1.31 min, [M+H]⁺=637.3; LC-MS Method F: RT=1.02 min, [M+H]⁺=637.3; ¹H NMR (400 MHz, DMSO-d₆) δ=8.34 (br s, 1H), 8.00 (s, 1H), 7.28 (br s, 1H), 7.13 (s, 1H), 6.81 (br s, 1H), 6.67-6.60 (m, 1H), 5.39 (d, J=1.3 Hz, 2H), 5.11 (br s, 1H), 4.19 (br s, 1H), 3.82-3.74 (m, 3H), 3.24-3.20 (m, 6H), 3.02-2.69 (m, 4H), 2.60 (s, 3H), 2.06-1.91 (m, 3H), 1.83 (br s, 2H), 1.74-1.45 (m, 6H), 1.42-1.29 (m, 2H), 1.16 (br s, 2H).

### Example 1003

### N—((R)-1-(4-amino-7-(5-oxo-2,5-dihydrofuran-3-yl)pyrrolo[2,1-f][1,2,4]triazin-5-yl)piperidin-3-yl)-2-methyl-4-(((1S,4S)-4-(pentan-3-ylamino)cyclohexyl)oxy)thiazole-5-carboxamide

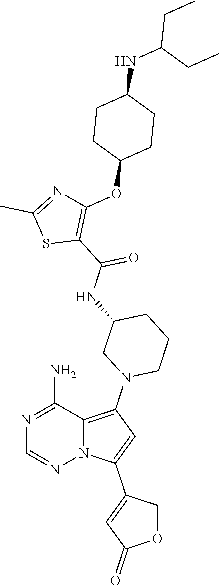 [SMILES: CCC(CC)N[C@H]1CC[C@@H](Oc2nc(C)sc2C(=O)N[C@@H]2CCCN(c3cc(C4=CC(=O)OC4)n4ncnc(N)c34)C2)CC1]

Analogous to the preparation of Example 996B, reaction of Example 1002E (40 mg, 0.06 mmol), sodium cyanoborohydride (7.54 mg, 0.12 mmol), acetic acid (3.43 μl, 0.06 mmol) and pentan-3-one (15.50 mg, 0.18 mmol) in methanol (1.5 mL) at rt for 1 h afforded the crude which was purified via reverse phase chromatography to afford Example 1003 (24.7 mg, 64% yield) as pale yellow solid. LC-MS Method E: RT=1.55 min, [M+H]⁺=623.3; LC-MS Method F: RT=1.17 min, [M+H]⁺=623.3; ¹H NMR (400 MHz, DMSO-d₆) δ=8.33 (br s, 1H), 7.99 (s, 1H), 7.29 (br s, 1H), 7.13 (s, 1H), 6.83 (br s, 1H), 6.64 (s, 1H), 6.53 (br s, 1H), 5.38 (s, 2H), 5.14 (br s, 1H), 4.18 (br s, 1H), 2.98 (br s, 1H), 2.78 (br s, 2H), 2.63-2.58 (m, 4H), 2.01-1.87 (m, 4H), 1.83 (br s, 2H), 1.74-1.49 (m, 5H), 1.41-1.14 (m, 7H), 0.75 (br t, J=6.9 Hz, 6H).

### Example 1004

### Preparation of N—((R)-1-(4-amino-7-(5-oxo-2,5-dihydrofuran-3-yl)pyrrolo[2,1-f][1,2,4]triazin-5-yl)piperidin-3-yl)-4-(((1S,4S)-4-(bis(methyl-d3)amino)cyclohexyl)oxy)-2-methylthiazole-5-carboxamide

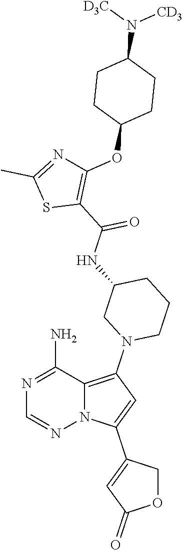 [SMILES: [2H]C([2H])([2H])N([C@H]1CC[C@@H](Oc2nc(C)sc2C(=O)N[C@@H]2CCCN(c3cc(C4=CC(=O)OC4)n4ncnc(N)c34)C2)CC1)C([2H])([2H])[2H]]

Analogous to the preparation of Example 996b, reaction of Example 1002E (100 mg, 0.18 mmol), formaldehyde-d₂ (145 mg, 0.90 mmol) and sodium cyanodeuteride (23.83 mg, 0.36 mmol) in CD₃OD (3 mL) afforded the crude which then purified via preparative.HPLC using Method 0 to afford Example 1004 (24 mg, 22% yield) as pale yellow solid. LC-MS Method K: RT=1.54 min, MS:[M−H]⁺=586.25; HPLC Method G: RT=6.12 min, Method H: RT=7.75 min; ¹H NMR (400 MHz, DMSO-d₆) δ=9.40-9.29 (m, 1H), 8.44-8.31 (m, 1H), 8.03-7.96 (m, 1H), 7.21-7.08 (m, 2H), 6.99-6.84 (m, 1H), 5.42-5.31 (m, 1H), 5.14-5.03 (m, 1H), 4.20-4.05 (m, 1H), 3.31-3.17 (m, 3H), 3.03-2.88 (m, 2H), 2.86-2.70 (m, 2H), 2.64-2.58 (m, 3H), 2.28-2.17 (m, 2H), 1.98-1.80 (m, 4H), 1.77-1.63 (m, 3H), 1.62-1.43 (m, 3H).

### Example 1005

### Preparation of (R)—N-(1-(4-amino-7-(4-methyl-5-oxo-2,5-dihydrofuran-3-yl)pyrrolo[2,1-f][1,2,4]triazin-5-yl)piperidin-3-yl)-2-methyl-4-((4-(tert-pentylamino)cyclohexyl)oxy)thiazole-5-carboxamide

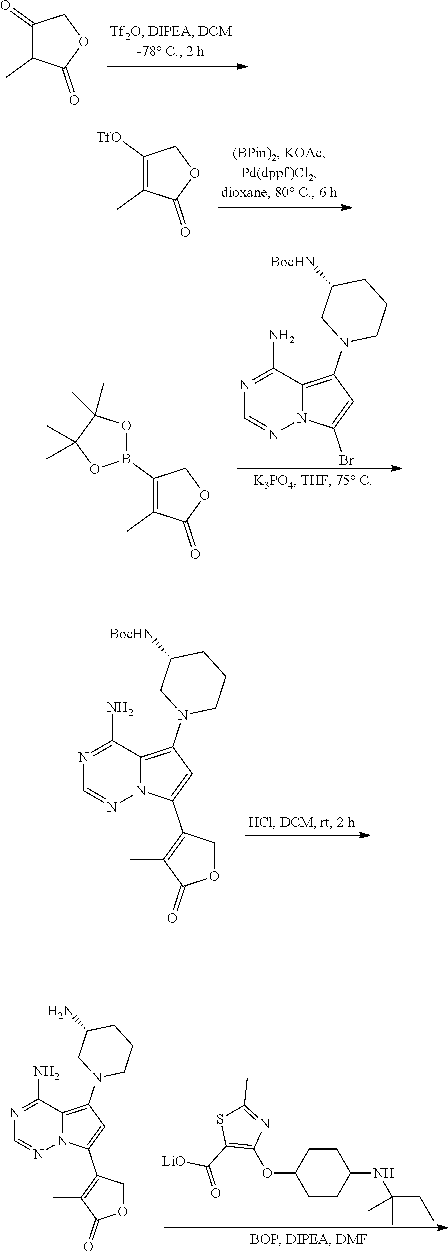

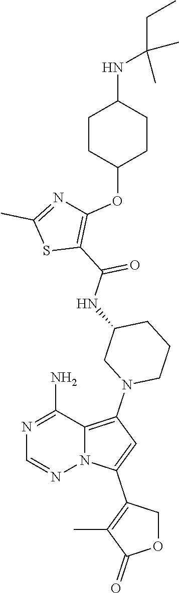 [SMILES: CCC(C)(C)NC1CCC(Oc2nc(C)sc2C(=O)N[C@@H]2CCCN(c3cc(C4=C(C)C(=O)OC4)n4ncnc(N)c34)C2)CC1]

### Example 1005A. Preparation of 4-methyl-5-oxo-2,5-dihydrofuran-3-yl trifluoromethanesulfonate

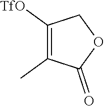

Analogous to the preparation of Example 996d, reaction of 4-hydroxy-3-methylfuran-2(5H)-one (2 g, 17.53 mmol), DIPEA (3.06 mL, 17.53 mmol) and trifluoromethanesulfonic anhydride (3.55 mL, 21.03 mmol) in DCM (50 mL) at −78° C. for 3 h to afforded Example 1005A (2 g, 46% yield) as yellow liquid; ¹H NMR (300 MHz, CHLOROFORM-d) δ ppm 4.90 (q, J=2.03 Hz, 2H), 1.95 (t, J=1.97 Hz, 3H).

### Example 1005B. Preparation of 3-methyl-4-(4,4,5,5-tetramethyl-1,3,2-dioxaborolan-2-yl)furan-2(5H)-one

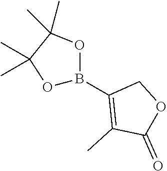 [SMILES: CC1=C(B2OC(C)(C)C(C)(C)O2)COC1=O]

Analogous to the preparation of Example 996e, reaction of Example 1005A (7.0 g, 28.4 mmol), bispin (7.94 g, 31.3 mmol), potassium acetate (3.35 g, 34.1 mmol), [1,1-bis(diphenylphosphino)ferrocene]dichloropalladium(II) (1.24 g, 1.70 mmol) in 1,4-dioxane (40 mL) at 80° C. for 6 h to afforded Example 1005B (12 g, 100% yield) as brown gummy solid, which was taken as such for next step. MS. [M+H]⁺=142.9 (Corresponding boronic acid mass).

### Example 1005C. Preparation of tert-butyl (R)-(1-(4-amino-7-(4-methyl-5-oxo-2,5-dihydrofuran-3-yl)pyrrolo[2,1-f][1,2,4]triazin-5-yl)piperidin-3-yl)carbamates

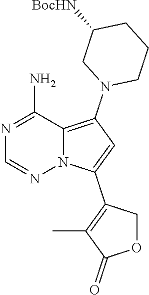 [SMILES: CC1=C(c2cc(N3CCC[C@@H](NC(=O)OC(C)(C)C)C3)c3c(N)ncnn23)COC1=O]

Analogous to the preparation of Example 996f, reaction of Example 1005B (12.26 g, 54.7 mmol), tert-butyl (R)-(1-(4-amino-7-bromopyrrolo[2,1-f][1,2,4]triazin-5-yl)piperidin-3-yl)carbamate (4.5 g, 10.94 mmol), potassium phosphate tribasic (10.94 mL, 21.88 mmol) and PdCl₂(dppf)-CH₂Cl₂ (0.89 g, 1.09 mmol) in THF (40 mL) at 75° C. for 6 h afforded the crude which was purified in column to afford Example 1005C (1.2 g, 26% yield) as yellow solid. MS. [M+H]⁺=429.5.

### Example 1005D. Preparation of (R)-4-(4-amino-5-(3-aminopiperidin-1-yl)pyrrolo[2,1-f][1,2,4]triazin-7-yl)-3-methylfuran-2(5H)-one. HCl

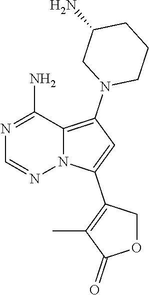 [SMILES: CC1=C(c2cc(N3CCC[C@@H](N)C3)c3c(N)ncnn23)COC1=O]

Analogous to the preparation of Example 996G, reaction of Example 1005C (3.0 g, 7.0 mmol) and HCl (4 M in dioxane) (17.50 mL, 70.0 mmol) in dioxane (10 mL) at it for 16 h afforded Example 1005D (2.5 g, 98% yield) as yellow solid. MS. [M+H]⁺=329.1; ¹H NMR (400 MHz, DMSO-d₆) δ ppm 8.64-8.40 (br s, 2H), 8.11 (s, 1H), 7.14 (s, 1H), 5.42 (br d, J=1.5 Hz, 2H), 5.09 (br s, 2H), 3.28 (br d, J=10.0 Hz, 1H), 3.19-2.95 (m, 2H), 2.81-2.67 (m, 2H), 2.09 (s, 3H), 1.90-1.64 (m, 4H).

### Example 1005

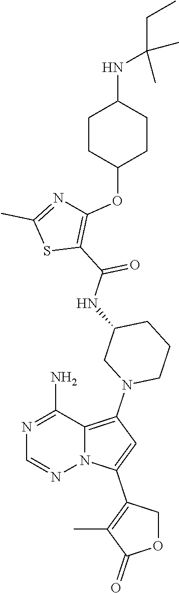 [SMILES: CCC(C)(C)NC1CCC(Oc2nc(C)sc2C(=O)N[C@@H]2CCCN(c3cc(C4=C(C)C(=O)OC4)n4ncnc(N)c34)C2)CC1]

Analogous to the preparation of Example 996, reaction of Example 1005D (77 mg, 0.21 mmol), lithium 2-methyl-4-((4-(tert-pentylamino)cyclohexyl)oxy)thiazole-5-carboxylate (70 mg, 0.21 mmol), BOP (112 mg, 0.25 mmol) and DIPEA (0.11 mL, 0.63 mmol) in DMF (3 mL) at RT for 2 h afforded Example 1005 (25 mg, 19% yield) as pale yellow solid. LC-MS Method E: RT=1.59 min, [M+H]+=637.3; LC-MS Method F: RT=1.11 min, [M+H]+=637.3. 1H NMR (400 MHz, DMSO-d6) δ=8.24 (s, 1H), 7.92 (s, 1H), 7.28 (br s, 1H), 7.07 (s, 1H), 6.87 (br s, 1H), 5.45 (d, J=1.8 Hz, 2H), 5.11 (br s, 1H), 4.20 (br s, 1H), 2.91 (br s, 2H), 2.60 (s, 3H), 2.07 (s, 3H), 2.02-1.89 (m, 4H), 1.85 (br d, J=3.8 Hz, 2H), 1.75-1.25 (m, 6H), 0.91 (br s, 6H), 0.71 (br t, J=6.5 Hz, 3H).

The following examples in Table 69 were prepared using the same procedure as shown in Example 1005.

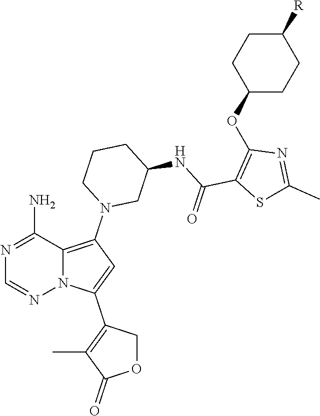

TABLE 69

| | | | | HPLC | |
|---|---|---|---|---|---|
| | Structure | | LCMS | Method, | |
| Example | R | Name | (M+H)+ | RT (min.) | HNMR |
| 1006 | | (R)-N-(1-(4-amino-7-(4- | 607.3 | A: 1.40 | (400 MHz, DMSO-d6) 8 = 8.51 - 8.36 (m, 1H), 7.92 (S, |
| | | methyl-5-oxo-2,5- | | B: 1.02 | IH), 7.38 - 7.22 (m, IH), 7.07 (s, 1H), 6.94 - 6.76 (m, |
| | | dihydrofuran-3- | | | IH), 5.46 (d, J = 2.0 Hz, 2H), 5.04 (br s, IH), 4.21 (br s, |
| | | yl)pyrrolo[2,1-f][ 1,2,4]triazin- | | | 1H), 3.13 - 2.77 (m, 5H), 2.60 (s, 3H), 2.11 - 2.02 (m, |
| | | 5-y1)piperidin-3-y1)-4-((4- | | | 4H), 1.99 - 1.79 (m, 8H), 1.65 (br s, 4H), 1.49 (br s, 2H), |
| | | (azetidin-1- | | | 1.38 - 1.22 (m, 3H) |
| | | yl)cyclohexyl)oxy)-2- | | | |
| | | methylthiazole-5-carboxamide | | | |
| 1007 | HN | N-((R)-1-(4-amino-7-(4- | 639.3 | A: 1.53 | (400 MHz, DMSO-d6) 8 = 8.18 (s, IH), 7.91 (s, 1H), 7.25 |
| | | methyl-5-oxo-2.5- | | B: 1.09 | (br s, IH), 7.06 (s, IH), 6.91 (br s, IH), 5.53 - 5.39 (m, |
| | | dihydrofuran-3- | | | 2H), 5.14 (br s, 1H), 4.18 (br s, 1H), 3.27 - 3.22 (m, 3H), |
| | | yl)pyrrolo[2,1-f][1,2,4]triazin- | | | 3.18 (s, 3H), 3.15 - 2.95 (m, 2H), 2.93 - 2.64 (m, 3H), |
| | | 5-y1)piperidin-3-y1)-4-((4- | | | 2.60 (s, 3H), 2.07 (t, J = 1.8 Hz, 3H), 2.02 - 1.78 (m,6H), |
| | | (((R)-1-methoxypropan-2- | | | 1.75 - 1.46 (m, 4H), 1.43 - 1.20 (m, 2H), 0.89 (br d, J = |
| | | yl)amino)cyclohexyl)oxy)-2~ | | | 6.3 Hz, 3H) |
| | | methylthiazole-5-carboxamide | | | |
| Example | Structure | Name | LCMS | HPLC | HNMR |
| | R | | (M+H)+ | Method, | |
| | | | | RT (min.) | |
| 1008 | OH | N-((R)-1-(4-amino-7-(4- | 639.3 | A: 1.53 | (400 MHz, DMSO-d6) 8 = 8.42 (br s, IH), 7.92 (s, 1H), |
| | | methyl-5-oxo-2,5- | | B: 1.09 | 7.36 (s, IH), 7.07 (s, IH), 6.85 (s, IH), 5.52 - 5.40 (m, |
| | | dihydrofuran-3- | | | 2H), 5.06 (br d, J = 2.8 Hz, IH), 4.27 (br s, 2H), 3.24 (br |
| | | y1)pyrrolo[2,1-f][ 1,2,4]triazin- | | | s, 2H), 2.92 (br s, 4H), 2.60 (s, 3H), 2.23 (br dd, J = 6.1, |
| | | 5-yl)piperidin-3-y1)-4-((4- | | | 6.9 Hz, 4H), 2.08 (t, J = 1.8 Hz, 3H), 2.01 - 1.73 (m, |
| | | (((R)-1-methoxypropan-2- | | | 6H), 1.69 - 1.32 (m, 3H), 1.32 - 1.18 (m, 2H), 0.96 - 0.81 |
| | | yl)amino)cyclohexyl)oxy)-2- | | | (m, 6H) |
| | | methylthiazole-5-carboxamide | | | |
| 1009 | OH | N-((R)-1-(4-amino-7-(4- | 639.3 | A: 1.53 | (400 MHz, DMSO-d6) 8 = 8.34 - 8.10 (m, 1H), 7.91 (s, |
| | HNS | methyl-5-oxo-2,5- | | B: 1.09 | IH), 7.34 - 7.20 (m, 1H), 7.06 (s, IH), 6.98 - 6.83 (m, |
| | | dihydrofuran-3- | | | IH), 5.46 (s, 2H), 5.14 (br s, 1H), 4.25 - 4.10 (m, 1H), |
| | | yl)pyrrolo[2, 1-f][1,2,4]triazin- | | | 3.27 - 3.11 (m, 2H), 3.08 - 2.95 (m, IH), 2.87 - 2.77 (m, |
| | | 5-y1)piperidin-3-y1)-4-((4- | | | IH), 2.72 - 2.64 (m, 2H), 2.60 (s, 3H), 2.07 (t, J = 1.8 Hz, |
| | | (((S)-1-hydroxybutan-2- | | | 4H), 1.96 (br s, 3H), 1.91 (s, 1H), 1.84 (br s, 2H), 1.76 - |
| | | yl)amino)cyclohexyl)oxy)-2- | | | 1.43 (m, 6H), 1.34 (br d, J = 6.0 Hz, 3H), 1.24 (s, 2H), |
| | | methylthiazole-5-carboxamide | | | 0.79 (br t, J = 7.3 Hz, 3H) |
| Example | Structure | Name | LCMS | HPLC | IHNMR |
| | R | | (M+H)+ | Method, | |
| | | | | RT (min.) | |
| 1010 | OH | N-((R)-1-(4-amino-7-(4- | 639.3 | A: 1.41 | (400 MHZ, DMSO-d6) ¿ = 8.35 - 8.20 (m, 1H), 7.91 (s, |
| | HN | methyl-5-oxo-2,5~ | | B: 1.06 | IH), 7.35 - 7.18 (m, 1H), 7.06 (s, 1H), 6.95 - 6.75 (m, |
| | | dihydrofuran~3- | | | IH), 5.46 (s, 2H), 5.15 (br s, IH), 4.22 - 4.07 (m, 1H), |
| | | yl)pyrrolo[2,1-f][ 1,2,4]triazin- | | | 2.99 (br s, IH), 2.90 - 2.79 (m, 1H), 2.68 - 2.63 (m, IH), |
| | | 5-y1)piperidin-3-y1)-4-((4- | | | 2.60 (s, 3H), 2.57 - 2.53 (m, 1H), 2.40 (br s, 1H), 2.07 (t, |
| | | (((R)-1-hydroxybutan-2- | | | J = 1.8 Hz, 3H), 1.94 (br d, J = 19.5 Hz, 4H), 1.84 (br s, |
| | | yl)amino)cyclohexyl)oxy)-2- | | | 2H), 1.67 (br s, 7H), 1.39 - 1.27 (m, 4H), 1.27 - 1.13 (m, |
| | | methylthiazole-5-carboxamide | | | 2H), 0.82 - 0.73 (m, 3H) |

### Example 1011

### Preparation of (R)—N-(1-(4-amino-7-(4-methyl-5-oxo-2,5-dihydrofuran-3-yl)pyrrolo[2,1-f][1,2,4]triazin-5-yl)piperidin-3-yl)-4-((4-(ethylamino)cyclohexyl)oxy)-2-methylthiazole-5-carboxamide

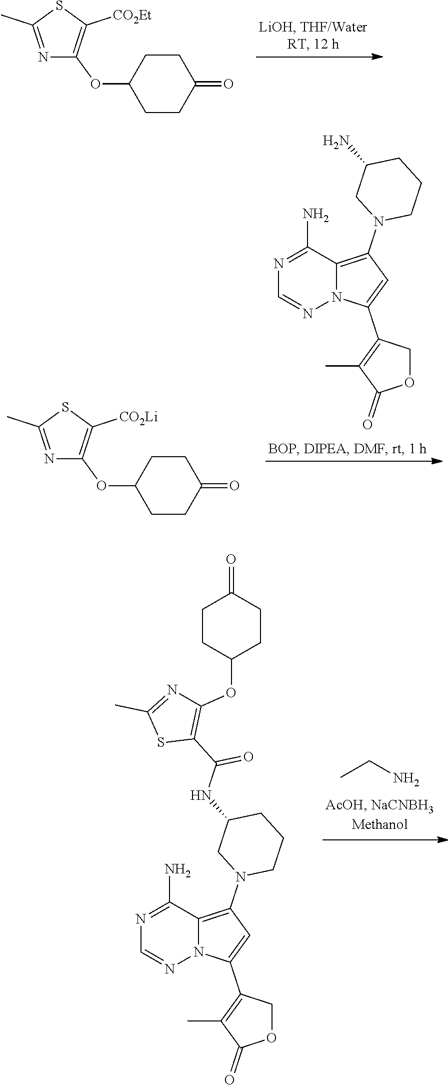

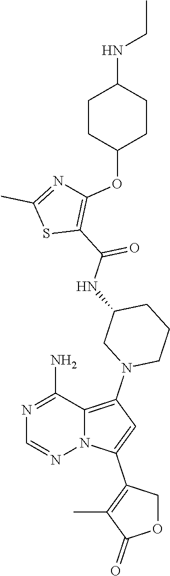 [SMILES: CCNC1CCC(Oc2nc(C)sc2C(=O)N[C@@H]2CCCN(c3cc(C4=C(C)C(=O)OC4)n4ncnc(N)c34)C2)CC1]

### Example 1011A. Preparation of lithium 2-methyl-4-((4-oxocyclohexyl)oxy)thiazole-5-carboxylate

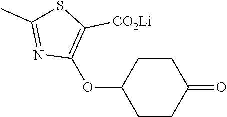

Analogous to the preparation of Example 996C, reaction of Example 941B (1 g, 3.53 mmol) and LiOH·1H₂O (0.25 g, 10.59 mmol) in ethanol (4 mL)\THF (5 mL)\Water (1 mL) at RT for 12 h to afforded Example 1011A (450 mg, 65% yield) as white solid. MS. [M+H]⁺=255.2.

### Example 1011B. Preparation of (R)—N-(1-(4-amino-7-(4-methyl-5-oxo-2,5-dihydrofuran-3-yl)pyrrolo[2,1-f][1,2,4]triazin-5-yl)piperidin-3-yl)-2-methyl-4-((4-oxocyclohexyl)oxy)thiazole-5-carboxamide

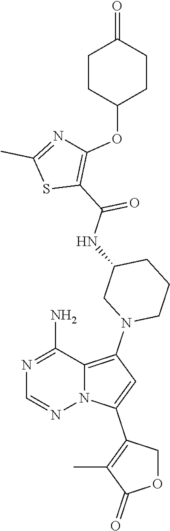 [SMILES: CC1=C(c2cc(N3CCC[C@@H](NC(=O)c4sc(C)nc4OC4CCC(=O)CC4)C3)c3c(N)ncnn23)COC1=O]

Analogous to the preparation of Example 9%, reaction of Example 1005D (400 mg, 0.90 mmol), Example 1011A (236 mg, 0.90 mmol), BOP (480 mg, 1.08 mmol) and DIPEA (0.79 mL, 4.52 mmol) in DMF (4 mL) at rt for 16 h afforded Example 1011B (350 mg, 68% yield) as yellow solid. MS. [M+H]⁺=566.10.

### Example 1011

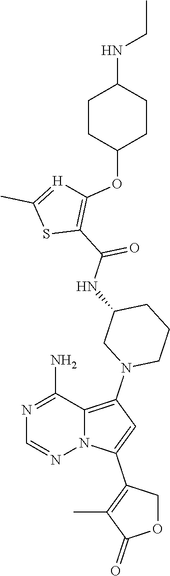

Analogous to the preparation of Example 996B, reaction of Example 1011B (150 mg, 0.26 mmol), ethanamine (23.91 mg, 0.53 mmol), acetic acid (7.59 μl, 0.13 mmol) and sodium cyanoborohydride (33.3 mg, 0.53 mmol) in MeOH (1 mL) at rt for 1 h afforded the crude which was purified via reverse phase chromatography to afford Example 1011 (4.1 mg, 2% yield) as pale yellow solid. LC-MS Method E: RT=1.31 min, [M+H]⁺=595.3; LC-MS Method F: RT=1.04 min, [M+H]⁺=595.3; ¹H NMR (400 MHz, DMSO-d₆) δ=7.91 (s, 1H), 7.32 (br s, 1H), 7.06 (s, 1H), 6.89 (s, 1H), 5.46 (d, J=1.8 Hz, 2H), 5.11 (br s, 1H), 4.20 (br s, 1H), 2.96 (br s, 3H), 2.63-2.58 (m, 3H), 2.46 (br s, 2H), 2.07 (s, 5H), 2.00-1.73 (m, 7H), 1.70-1.50 (m, 3H), 1.43-1.29 (m, 2H), 0.91 (t, J=7.1 Hz, 3H).

### Example 1012

### Preparation of N—((R)-1-(4-amino-7-(4-methyl-5-oxo-2,5-dihydrofuran-3-yl)pyrrolo[2,1-f][1,2,4]triazin-5-yl)piperidin-3-yl)-4-(((1S,4S)-4-((2,2-dimethyltetrahydrofuran-3-yl)amino)cyclohexyl)oxy)-2-methylthiazole-5-carboxamide

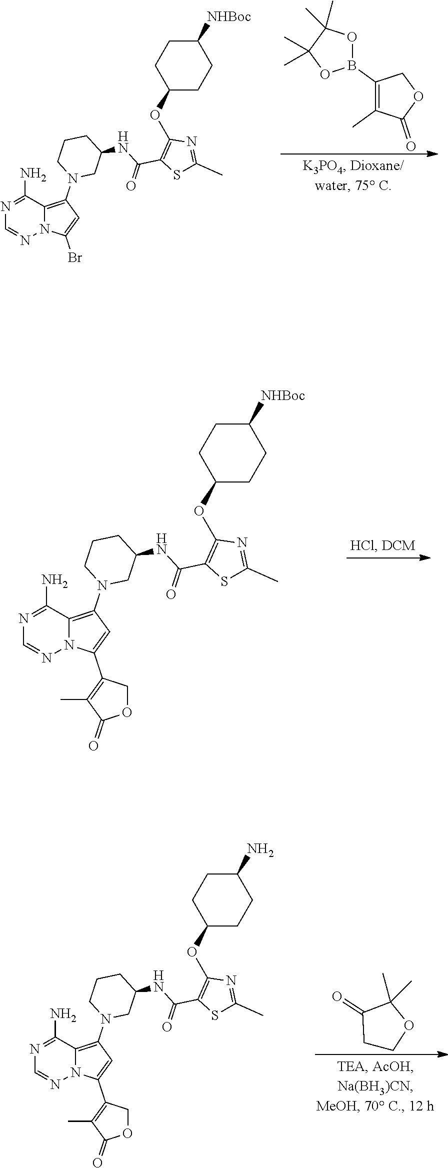

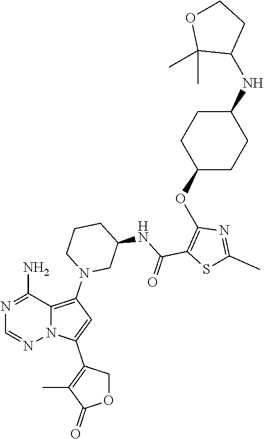 [SMILES: CC1=C(c2cc(N3CCC[C@@H](NC(=O)c4sc(C)nc4O[C@H]4CC[C@@H](NC5CCOC5(C)C)CC4)C3)c3c(N)ncnn23)COC1=O]

### Example 1012A. Preparation of tert-butyl ((1S,4S)-4-((5-(((R)-1-(4-amino-7-(4-methyl-5-oxo-2,5-dihydrofuran-3-yl)pyrrolo[2,1-f][1,2,4]triazin-5-yl)piperidin-3-yl)carbamoyl)-2-methylthiazol-4-yl)oxy)cyclohexyl)carbamate

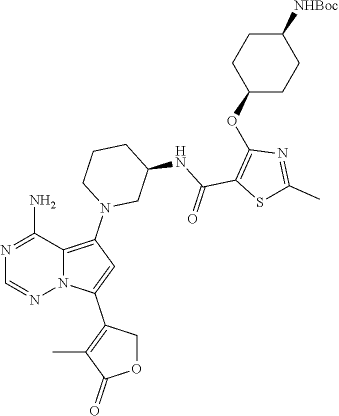

Analogous to the preparation of Example 996F, reaction of Example 1002C (1.5 g, 2.30 mmol), Example 1005B (2.59 g, 11.55 mmol), PdCl2(dppf)-CH2Cl2 adduct (0.37 g, 0.46 mmol) and potassium phosphate tribasic (1.47 g, 6.93 mmol) in dioxane (30 mL)/water (1 mL) at 85° C. for 6 h afforded the crude which was purified by column chromatography to get Example 1012A (1.2 g, 1.80 mmol, 78% yield) as an off-white solid. MS [M+H]⁺=667.2

### Example 1012B. Preparation of N—((R)-1-(4-amino-7-(4-methyl-5-oxo-2,5-dihydrofuran-3-yl)pyrrolo[2,1-f][1,2,4]triazin-5-yl)piperidin-3-yl)-4-(((1S,4S)-4-aminocyclohexyl)oxy)-2-methylthiazole-5-carboxamide. HCl

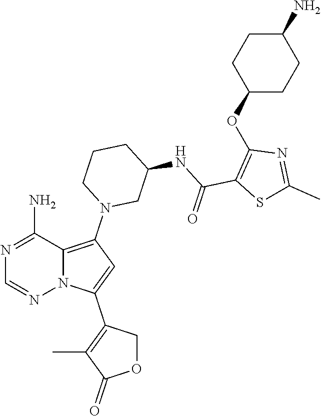 [SMILES: CC1=C(c2cc(N3CCC[C@@H](NC(=O)c4sc(C)nc4O[C@H]4CC[C@@H](N)CC4)C3)c3c(N)ncnn23)COC1=O]

Analogous to the preparation of Example 996G, reaction of Example 1012A (1.2 g, 1.80 mmol) and 4N HCl in dioxane (11.25 mL, 45.0 mmol) in DCM (20 mL) at rt for 3 h afforded the crude Example 1012B (950 mg, 88% yield) which was used in the next step without further purification. MS=567.2.

### Example 1012

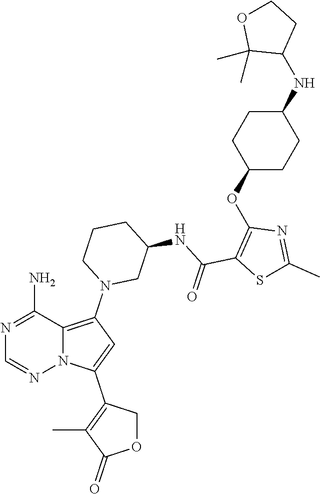 [SMILES: CC1=C(c2cc(N3CCC[C@@H](NC(=O)c4sc(C)nc4O[C@H]4CC[C@@H](NC5CCOC5(C)C)CC4)C3)c3c(N)ncnn23)COC1=O]

Analogous to the preparation of Example 996B, reaction of Example 1012B (0.1 g, 0.17 mmol), 2,2-dimethyldihydrofuran-3(2H)-one (0.04 g, 0.35 mmol), acetic acid (0.05 mL, 0.88 mmol), TEA (0.05 mL, 0.35 mmol) and sodium cyanoborohydride (0.03 g, 0.53 mmol) at 70° C. for 12 h afforded the crude diastereomer mixture which was separated by chiral PO method using method V to afford Example 1012 (Iso-1) (3.34 mg, 3% yield). LC-MS Method K: RT=1.26 min, [M+H]⁺=665.35; ¹H NMR (400 MHz, DMSO-d₆) δ=8.38-8.17 (m, 1H), 7.92-7.91 (m, 1H), 7.94-7.81 (m, 1H), 7.43-7.18 (m, 1H), 7.12-7.01 (m, 1H), 6.98-6.74 (m, 1H), 5.51-5.34 (m, 2H), 5.18-5.10 (m, 1H), 4.24-4.10 (m, 1H), 3.65-3.57 (m, 2H), 3.55-3.49 (m, 2H), 3.08-2.97 (m, 3H), 2.81-2.72 (m, 3H), 2.63-2.59 (m, 3H), 2.34 (br s, 1H), 2.11-2.03 (m, 3H), 2.01-1.91 (m, 2H), 1.88-1.79 (m, 2H), 1.75-1.57 (m, 3H), 1.55-1.46 (m, 1H), 1.39 (br s, 3H), 1.06-1.00 (m, 3H), 0.86-0.79 (m, 3H).

### Example 1013

### Preparation of N—((R)-1-(4-amino-7-(4-methyl-5-oxo-2,5-dihydrofuran-3-yl)pyrrolo[2,1-f][1,2,4]triazin-5-yl)piperidin-3-yl)-4-(((1S,4S)-4-(bis(methyl-d3)amino)cyclohexyl)oxy)-2-methylthiazole-5-carboxamide

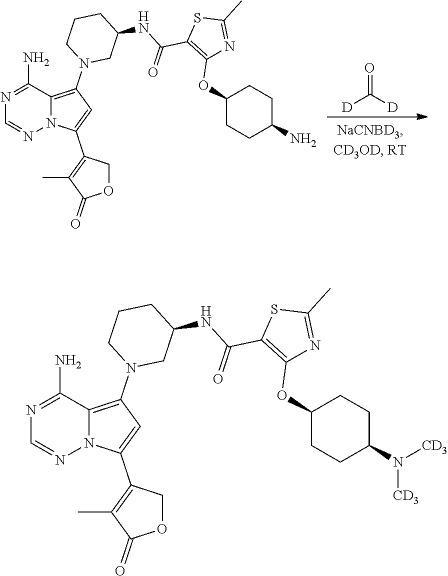

Analogous to the preparation of Example 996B, reaction of Example 1012B (100 mg, 0.17 mmol), formaldehyde-d₂ (141 mg, 0.88 mmol) and sodium cyanodeuteride (23.24 mg, 0.35 mmol) in CD₃OD (3 mL) at rt afforded the crude which purified via prep.HPLC using Method 0 yielded Example 1013 (24 mg, 22% yield) as pale yellow solid. LC-MS Method K: RT=1.68 min, [M+H]⁺=601.25; HPLC Method G: RT=6.62 min, Method H: RT=8.21 min; ¹H NMR (400 MHz, DMSO-d₆) δ 9.53-9.36 (m, 1H), 8.50-8.33 (m, 1H), 8.01-7.83 (m, 1H), 7.32-6.92 (m, 3H), 5.50-5.43 (m, 2H), 5.14-5.06 (m, 1H), 4.22-4.12 (m, 1H), 3.34-3.17 (m, 2H), 3.07-2.75 (m, 3H), 2.63-2.57 (m, 3H), 2.29-2.18 (m, 2H), 2.12-2.03 (m, 3H), 1.98-1.77 (m, 4H), 1.76-1.43 (m, 6H).

### Example 1014

### Preparation of N—((R)-1-(4-amino-7-(5-oxo-2,5-dihydrofuran-3-yl)pyrrolo[2,1-f][1,2,4]triazin-5-yl)piperidin-3-yl)-4-(((1S,4S)-4-(bis(methyl-d₃)amino)cyclohexyl)oxy)-2-(methyl-d3)thiazole-5-carboxamide

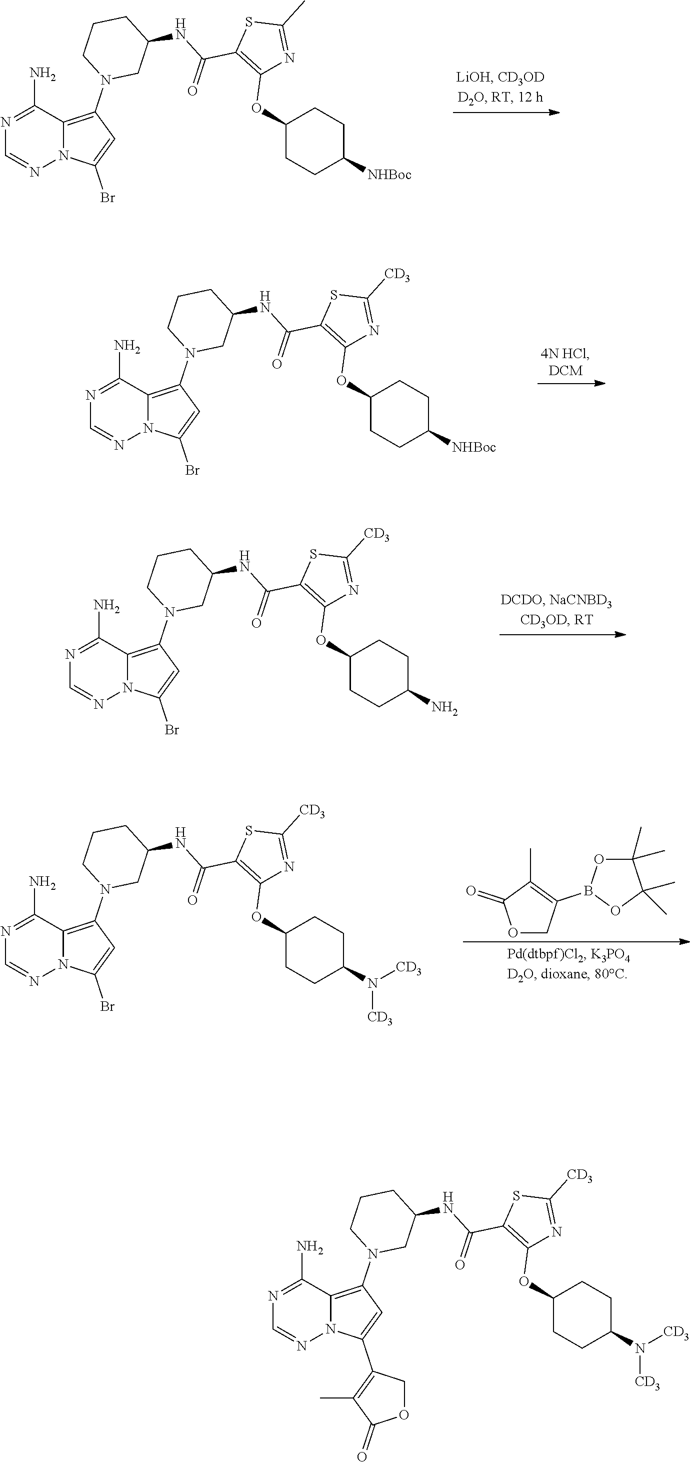

### Example 1014A. Preparation of tert-Butyl ((1S,4S)-4-((5-(((R)-1-(4-amino-7-bromopyrrolo[2,1-f][1,2,4]triazin-5-yl)piperidin-3-yl)carbamoyl)-2-(methyl-d₃)thiazol-4-yl)oxy)cyclohexyl)carbamate

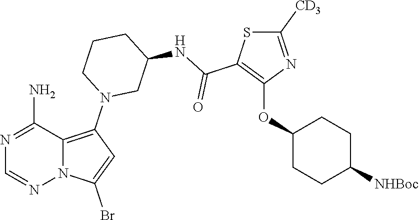 [SMILES: [2H]C([2H])([2H])c1nc(O[C@H]2CC[C@@H](NC(=O)OC(C)(C)C)CC2)c(C(=O)N[C@@H]2CCCN(c3cc(Br)n4ncnc(N)c34)C2)s1]

To a stirred solution of tert-butyl ((1S,4S)-4-((5-(((R)-1-(4-amino-7-bromopyrrolo[2,1-f][1,2,4]triazin-5-yl)piperidin-3-yl)carbamoyl)-2-methylthiazol-4-yl)oxy)cyclohexyl)carbamate (1.0 g, 1.54 mmol) in CD₃OD (5 mL)/THF (10 mL) was added a solution of LiOH (0.11 g, 4.62 mmol) in D₂O (5 mL) and the reaction mixture was stirred at rt for 12 h. The reaction mixture was concentrated in vacuo and the residue was diluted with EtOAc (20 mL), then washed with brine (1×20 mL) and dried to afford Example 1014A (920 mg, 92% yield) as a light-yellow solid. LCMS Method: MS: [M+H]₌652.2; ¹H NMR (400 MHz, METHANOL-d₄) δ=7.79-7.70 (m, 1H), 6.76-6.72 (m, 1H), 5.25-5.18 (m, 1H), 4.31-4.16 (m, 1H), 3.88-3.70 (m, 1H), 3.52-3.37 (m, 2H), 3.14-2.85 (m, 2H), 2.20-2.00 (m, 2H), 1.99-1.76 (m, 5H), 1.73-1.57 (m, 5H), 1.49-1.43 (m, 9H).

### Example 1014B. Preparation of N—((R)-1-(4-amino-7-bromopyrrolo[2,1-f][1,2,4]triazin-5-yl)piperidin-3-yl)-4-(((1S,4S)-4-aminocyclohexyl)oxy)-2-(methyl-d₃)thiazole-5-carboxamide. HCl

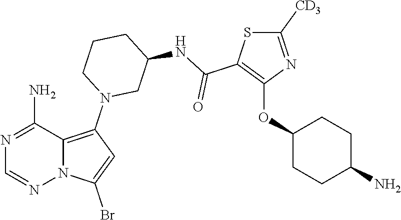 [SMILES: [2H]C([2H])([2H])c1nc(O[C@H]2CC[C@@H](N)CC2)c(C(=O)N[C@@H]2CCCN(c3cc(Br)n4ncnc(N)c34)C2)s1]

Analogous to the preparation of Example 996G, reaction of Example 1014A (500 mg, 0.76 mmol) and 4N HCl in dioxane (4.79 mL, 19.15 mmol) in DCM (10 mL) afforded Example 1014B (402 mg, 89% yield). MS: [M+H]⁺=552.2; ¹H NMR (400 MHz, METHANOL-d₄) δ=7.80-7.72 (m, 1H), 6.76-6.70 (m, 1H), 5.25-5.17 (m, 1H), 4.30-4.18 (m, 1H), 3.39-3.36 (m, 1H), 3.11-3.02 (m, 1H), 3.00-2.80 (m, 3H), 2.21-2.12 (m, 2H), 2.11-2.01 (m, 1H), 2.01-1.86 (m, 3H), 1.85-1.70 (m, 4H), 1.58-1.44 (m, 2H).

### Example 1014C. Preparation of N—((R)-1-(4-amino-7-bromopyrrolo[2,1-f][1,2,4]triazin-5-yl)piperidin-3-yl)-4-(((1S,4S)-4-(bis(methyl-d₃)amino)cyclohexyl)oxy)-2-(methyl-d3)thiazole-5-carboxamide

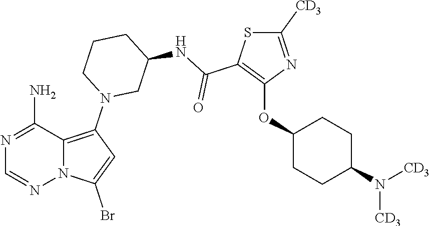 [SMILES: [2H]C([2H])([2H])c1nc(O[C@H]2CC[C@@H](N(C([2H])([2H])[2H])C([2H])([2H])[2H])CC2)c(C(=O)N[C@@H]2CCCN(c3cc(Br)n4ncnc(N)c34)C2)s1]

Analogous to the preparation of Example 996B, reaction of Example 1014B (250 mg, 0.42 mmol), formaldehyde-d₂ (340 mg, 2.12 mmol) and sodium cyanoborodeuteride (55.9 mg, 0.85 mmol) in CD₃OD (5 mL) afforded the crude which was diluted with saturated sodium bicarbonate solution and extracted with DCM (3×20 mL). The combined organic layer was washed with brine, dried over Na₂SO₄, filtered and concentrated under vacuum to obtain Example 1014C (210 mg, 0.36 mmol, 84% yield). MS: [M+H]⁺=586.3; ¹H NMR (400 MHz, METHANOL-d₄) δ=7.81-7.75 (m, 1H), 6.75-6.66 (m, 1H), 5.27-5.17 (m, 1H), 4.31-4.19 (m, 1H), 3.42-3.36 (m, 1H), 3.14-2.81 (m, 3H), 2.63-2.57 (m, 1H), 2.43-2.28 (m, 1H), 2.28-2.15 (m, 2H), 2.00-1.86 (m, 2H), 1.82-1.64 (m, 5H), 1.63-1.50 (m, 2H).

### Example 1014

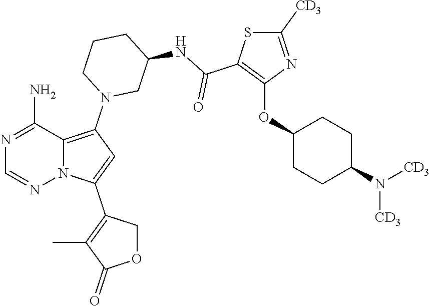 [SMILES: [2H]C([2H])([2H])c1nc(O[C@H]2CC[C@@H](N(C([2H])([2H])[2H])C([2H])([2H])[2H])CC2)c(C(=O)N[C@@H]2CCCN(c3cc(C4=C(C)C(=O)OC4)n4ncnc(N)c34)C2)s1]

Analogous to the preparation of Example 996F, reaction of Example 1014C (80 mg, 0.13 mmol), Example 1005B (122 mg, 0.54 mmol), potassium phosphate tribasic (87 mg, 0.41 mmol) and [1,1′-bis(di-tert-butylphosphino)ferrocene]dichloropalladium(II) (10.67 mg, 0.01 mmol) in dioxane (5 mL)/D₂O (0.5 mL) afforded the crude which subsequently purified using preparative LC/MS using Method L to afford Example 1014 (22.7 mg, 27% yield). LC-MS Method E: RT=1.33 min, MS: [M+H]⁺=604.3; LC-MS Method F: RT=1.00 min, MS: [M+H]⁺=604.3; ¹H NMR (400 MHz, DMSO-d₆) δ=8.33 (br s, 1H), 7.92 (s, 1H), 7.19 (br s, 1H), 7.05 (s, 1H), 6.93 (br s, 1H), 5.47 (d, J=1.5 Hz, 2H), 5.11 (br s, 1H), 4.18 (br s, 1H), 3.25 (br s, 2H), 2.98 (br s, 2H), 2.45 (br s, 1H), 2.15 (br s, 2H), 2.07 (s, 3H), 1.98-1.75 (m, 4H), 1.73-1.45 (m, 6H).

### Example 1015

### Preparation of N—((R)-1-(4-amino-7-(5-oxo-2,5-dihydrofuran-3-yl)pyrrolo[2,1-f][1,2,4]triazin-5-yl)piperidin-3-yl)-4-(((1S,4S)-4-(bis(methyl-d₃)amino)cyclohexyl)oxy)-2-(methyl-d₃)thiazole-5-carboxamide

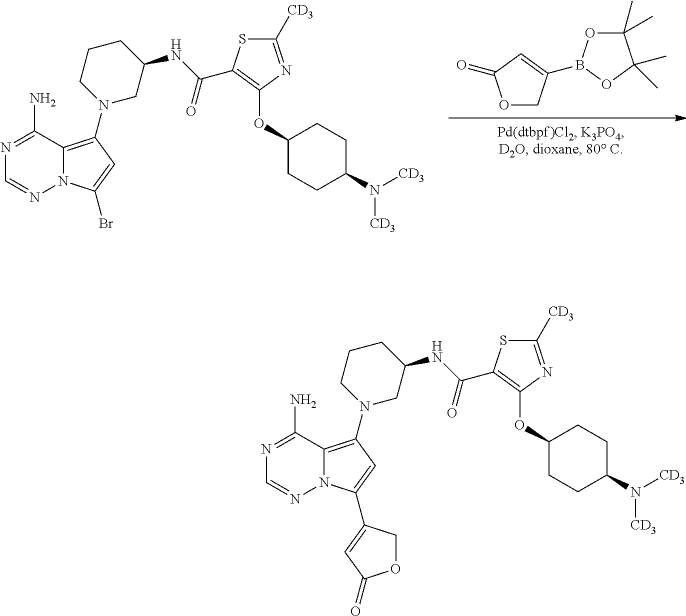

Analogous to the preparation of Example 996F, reaction of Example 1014C (80 mg, 0.13 mmol), Example 996E (115 mg, 0.54 mmol), potassium phosphate tribasic (87 mg, 0.41 mmol) and [1,1′-bis(di-tert-butylphosphino)ferrocene]dichloropalladium(II) (10.67 mg, 0.02 mmol) in dioxane (5 mL)/D₂O (0.5 mL), afforded the crude which was purified via preparative LC/MS using Method L yielded Example 1015 (12 mg, 14% yield). LC-MS Method E: RT=1.22 min, MS: [M+H]⁺=590.3; LC-MS Method F: RT=0.95 min, MS: [M+H]⁺=590.3; ¹H NMR (400 MHz, DMSO-d₆) δ=8.38 (br s, 1H), 8.01 (s, 1H), 7.22 (br s, 1H), 7.13 (s, 1H), 6.87 (br s, 1H), 6.65 (t, J=1.5 Hz, 1H), 5.40 (d, J=1.3 Hz, 2H), 5.12 (br s, 1H), 4.17 (br s, 1H), 3.23 (br s, 3H), 2.98 (br s, 1H), 2.68 (s, 1H), 2.15 (br s, 2H), 1.97-1.80 (m, 3H), 1.77-1.60 (m, 4H), 1.60-1.43 (m, 3H).

### Example 1016

### Preparation of N—((R)-1-(4-amino-7-(4-methyl-5-oxo-2,5-dihydrofuran-3-yl)pyrrolo[2,1-f][1,2,4]triazin-5-yl)piperidin-3-yl)-4-(((1S,4S)-4-(dimethylamino)cyclohexyl)oxy)-2-(methyl-d₃)thiazole-5-carboxamide

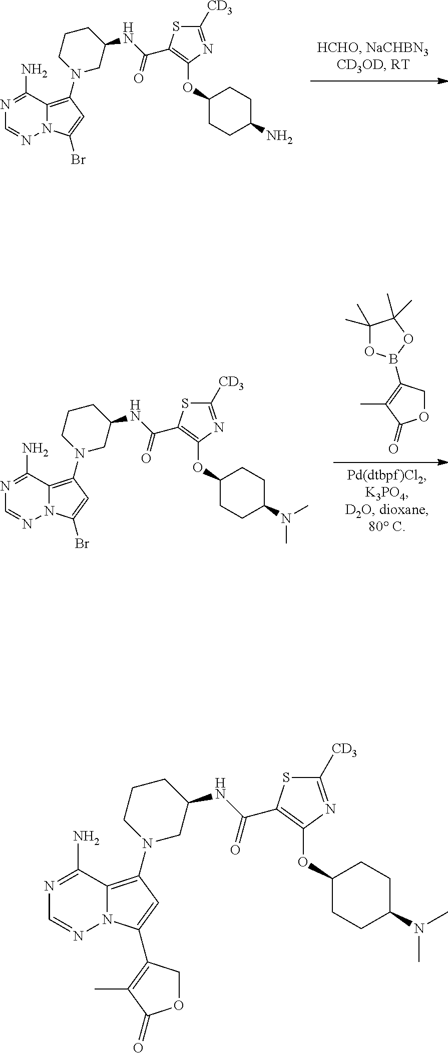

### Example 1016A. Preparation of N—((R)-1-(4-amino-7-bromopyrrolo[2,1-f][1,2,4]triazin-5-yl)piperidin-3-yl)-4-(((1S,4S)-4-(dimethylamino)cyclohexyl)oxy)-2-(methyl-d₃)thiazole-5-carboxamide

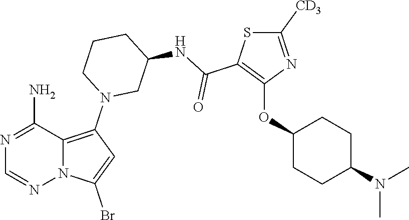 [SMILES: [2H]C([2H])([2H])c1nc(O[C@H]2CC[C@@H](N(C)C)CC2)c(C(=O)N[C@@H]2CCCN(c3cc(Br)n4ncnc(N)c34)C2)s1]

Analogous to the preparation of Example 996B, reaction of Example 1014B (300 mg, 0.51 mmol), formaldehyde (201 mg, 2.55 mmol) and sodium cyanoborohydride (96 mg, 1.53 mmol) in CD₃OD (10 mL) afforded the crude which was diluted with saturated sodium bicarbonate solution and extracted with DCM (3×20 mL). The combined organic layer was washed with brine, dried over Na₂SO₄, filtered and concentrated under vacuum to obtain Example 1016A (240 mg, 81% yield). MS: [M−H]⁺=578.3.

### Example 1016

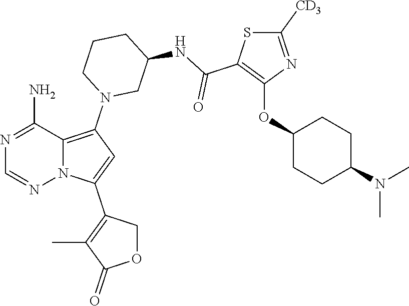 [SMILES: [2H]C([2H])([2H])c1nc(O[C@H]2CC[C@@H](N(C)C)CC2)c(C(=O)N[C@@H]2CCCN(c3cc(C4=C(C)C(=O)OC4)n4ncnc(N)c34)C2)s1]

Analogous to the preparation of Example 996F, reaction of Example 1016A (100 mg, 0.17 mmol), Example 1005B (154 mg, 0.69 mmol), potassium phosphate tribasic (110 mg, 0.52 mmol) and [1,1′-bis(di-tert-butylphosphino)ferrocene]dichloropalladium(II) (13.47 mg, 0.02 mmol) in dioxane (5 mL)/D₂O (0.5 mL) afforded the crude which was purified via preparative LC/MS using Method L yielded Example 1016 (10.4 mg, 10% yield). LC-MS Method E: RT=1.34 min, MS: [M+H]⁺=598.3; LC-MS Method F: RT=1.0 min, MS: [M+H]⁺=598.3; ¹H NMR (400 MHz, DMSO-d₆) δ=8.36 (br s, 1H), 7.92 (s, 1H), 7.24 (br s, 1H), 7.05 (s, 1H), 6.92 (br s, 1H), 5.46 (br s, 2H), 5.12 (br s, 1H), 4.19 (br s, 1H), 3.25 (br s, 2H), 2.98 (br s, 2H), 2.30-1.95 (m, 1H), 1.84 (br s, 2H), 1.73-1.45 (m, 6H), 1.32-1.00 (m, 3H).

### Example 1017

### Preparation of N—((R)-1-(4-amino-7-(4-methyl-5-oxo-2,5-dihydrofuran-3-yl) pyrrolo[2,1-f][1,2,4]triazin-5-yl)piperidin-3-yl)-4-(((1S,4S)-4-(dimethylamino)cyclohexyl)oxy)-2-(methyl-d₃)thiazole-5-carboxamide

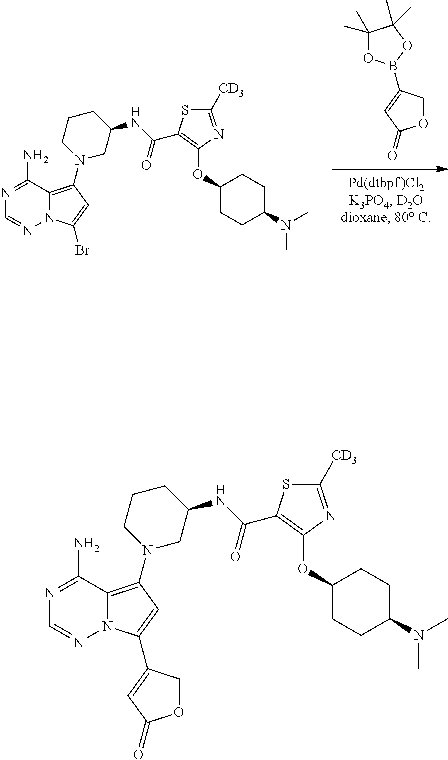 [SMILES: [2H]C([2H])([2H])c1nc(O[C@H]2CC[C@@H](N(C)C)CC2)c(C(=O)N[C@@H]2CCCN(c3cc(Br)n4ncnc(N)c34)C2)s1 ; CC1(C)OB(C2=CC(=O)OC2)OC1(C)C ; "Pd(dtbpf)Cl2K3PO4, D2Odioxane, 80° C." ; [2H]C([2H])([2H])c1nc(O[C@H]2CC[C@@H](N(C)C)CC2)c(C(=O)N[C@@H]2CCCN(c3cc(C4=CC(=O)OC4)n4ncnc(N)c34)C2)s1]

Analogous to the preparation of Example 996F, Example 1016A (120 mg, 0.21 mmol), Example 996E (174 mg, 0.83 mmol), potassium phosphate tribasic (132 mg, 0.62 mmol) and [1,1′-bis(di-tert-butylphosphino)ferrocene]dichloropalladium(II) (16.17 mg, 0.02 mmol) in a mixture of dioxane (6 mL) and D₂O (1 mL) afforded the crude which then purified via preparative LC/MS using Method L yielded Example 1017 (30.6 mg, 25% yield). LC-MS Method E: RT=1.23 min, MS: [M+H]⁺=585.3; LC-MS Method F: RT=0.94 min, MS: [M+H]⁺=585.3; ¹H NMR (400 MHz, DMSO-d₆) δ=8.35 (br s, 1H), 8.01 (s, 1H), 7.18 (br s, 1H), 7.13 (s, 1H), 6.90 (br s, 1H), 6.65 (d, J=1.3 Hz, 1H), 5.48-5.32 (m, 2H), 5.11 (br s, 1H), 4.30-3.99 (m, 1H), 3.24 (br d, J=9.5 Hz, 2H), 3.02-2.90 (m, 1H), 2.86-2.55 (m, 8H), 2.31-2.15 (m, 2H), 2.00-1.81 (m, 4H), 1.77-1.49 (m, 6H).

### Example 1018

### Preparation of N—((R)-1-(4-amino-7-(4-ethyl-5-oxo-2,5-dihydrofuran-3-yl)pyrrolo[2,1-f][1,2,4]triazin-5-yl)piperidin-3-yl)-4-(((1S,4S)-4-(dimethylamino)cyclohexyl)oxy)-2-methylthiazole-5-carboxamide

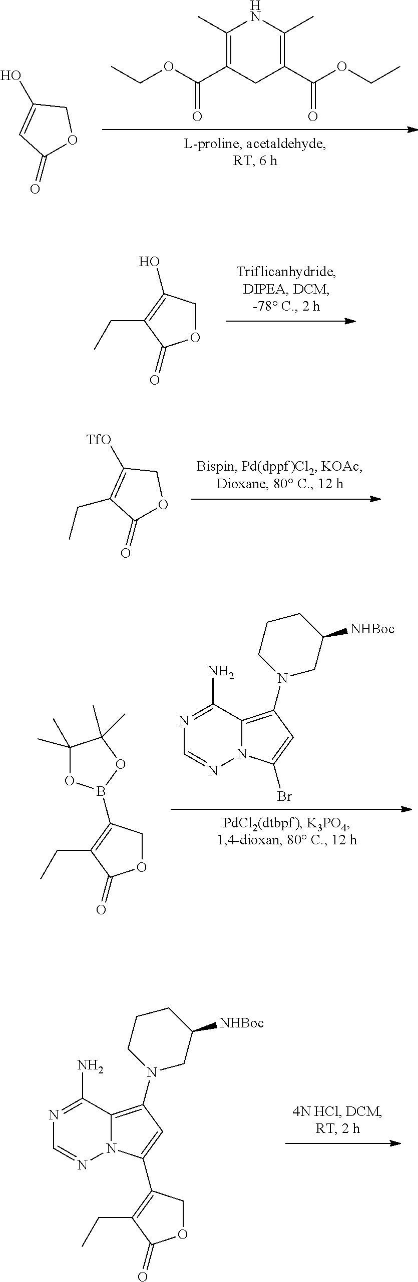

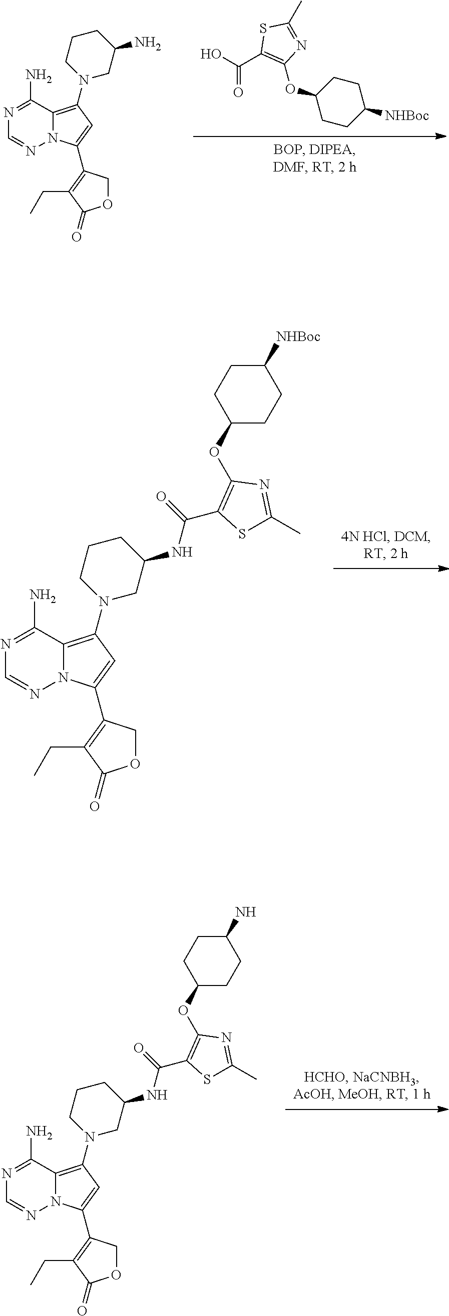

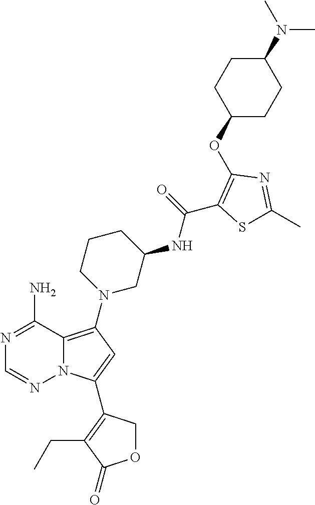 [SMILES: CCC1=C(c2cc(N3CCC[C@@H](NC(=O)c4sc(C)nc4O[C@H]4CC[C@@H](N(C)C)CC4)C3)c3c(N)ncnn23)COC1=O]

### Example 1018A. Preparation of 3-Ethyl-4-hydroxyfuran-2(5H)-one

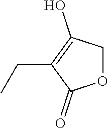

To a stirred solution of 4-hydroxyfuran-2(5H)-one (5 g, 50.0 mmol) in acetaldehyde (20 ml, 177 mmol), diethyl 1,4-dihydro-2,6-dimethyl-3,5-pyridinedicarboxylate (12.6 g, 50.0 mmol) was added L-Proline (0.06 g, 0.50 mmol) at rt and stirred for 6 h. The reaction mixture was concentrated under vacuum to get the crude which was purified by using CombiFlash (RedisepRf silica gel 80 g column; 0-15% ethyl acetate in pet-ether as an eluent) to afford Example 1018A (3.5 g, 55% yield) as yellow solid; MS: [M+H]⁺=129.1.

### Example 1019B. Preparation of 2-Ethyl-5-oxo-2,5-dihydrofuran-3-yl trifluoromethanesulfonate

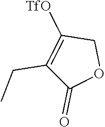 [SMILES: CCC1=C(OS(=O)(=O)C(F)(F)F)COC1=O]

To a stirred solution of Example 1018A (3.2 g, 12.30 mmol) in DCM (40 mL) was added DIPEA (7.16 mL, 41.0 mmol) followed by the addition of triflicanhydride (5.5 mL, 32.8 mmol) dropwise at −78° C. The reaction mixture was stirred at same temperature for 2 h and cold water was added dropwise over 10 min and stirred for another 30 min. The reaction suspension was extracted with DCM (2×50 mL). The combined organic layer was washed with brine (1-30 mL), dried over Na₂SO₄, filtered and concentrated under vacuum to get crude product which was triturated with hexane to get Example 1018B (3.2 g, 45% yield); ¹H NMR (300 MHz, CHLOROFORM-d) δ=4.93-4.85 (m, 2H), 2.46-2.30 (m, 3H), 1.22-1.15 (m, 3H).

### Example 1018C. Preparation of 3-Ethyl-4-(4,4,5,5-tetramethyl-1,3,2-dioxaborolan-2-yl) furan-2(5H)-one

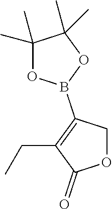 [SMILES: CCC1=C(B2OC(C)(C)C(C)(C)O2)COC1=O]

Analogous to the preparation of Example 996E, reaction of Example 1018B (2.5 g, 9.61 mmol), bispin (3.66 g, 14.41 mmol), potassium acetate (2.83 g, 28.8 mmol) and Pd(dppf)Cl₂·DCM (0.39 g, 0.48 mmol) in 1,4-dioxane (40 mL) afforded Example 1018C (2.2 g, 96% yield). The crude was used in the next step without further purification (2.2 g, 96% yield); ¹H NMR (300 MHz, CHLOROFORM-d) δ=4.99-4.95 (m, 2H), 2.44-2.39 (m, 2H), 1.24-1.23 (m, 9H), 1.06-1.03 (m, 3H).

### Example 1018D. Preparation of tert-butyl (R)-(1-(4-amino-7-(4-ethyl-5-oxo-2,5-dihydrofuran-3-yl) pyrrolo[2,1-f][1,2,4]triazin-5-yl)piperidin-3-yl)carbamate

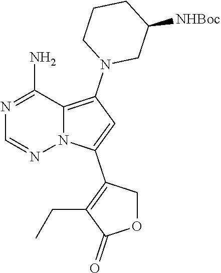 [SMILES: CCC1=C(c2cc(N3CCC[C@@H](NC(=O)OC(C)(C)C)C3)c3c(N)ncnn23)COC1=O]

Analogous to the preparation of Example 996F, reaction of tert-butyl (R)-(1-(4-amino-7-bromopyrrolo[2,1-f][1,2,4]triazin-5-yl)piperidin-3-yl)carbamate (2 g, 4.86 mmol), Example 1018C (10.42 g, 43.8 mmol), potassium phosphate tribasic (3.10 g, 14.59 mmol) and Pd(dppf)Cl₂·DCM (0.39 g, 0.48 mmol) in dioxane (40 mL)/water (4.44 mL) at 80° C. for 12 h afforded crude which was purified by using Combi Flash (RedisepRf silica gel 80 g column; 0-40% ethyl acetate in pet-ether as an eluent) to Example 1018D (0.85 g, 40% yield) as yellow solid: MS: [M+H]⁺=443.5.

### Example 1018E. Preparation of (R)-4-(4-Amino-5-(3-aminopiperidin-1-yl) pyrrolo[2,1-f][1,2,4]triazin-7-yl)-3-ethylfuran-2(5H)-one. HCl

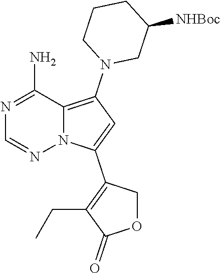 [SMILES: CCC1=C(c2cc(N3CCC[C@@H](NC(=O)OC(C)(C)C)C3)c3c(N)ncnn23)COC1=O]

Analogous to the preparation of Example 96G, reaction of Example 1018D (0.85 g, 1.92 mmol)) with 4M HCl in dioxane (2.40 ml, 9.60 mmol) in DCM (10 mL) at rt for 2 h afforded Example 1018E (0.71 g, 98% yield); MS: [M+H]⁺=343.3.

### Example 1018F. Preparation of tert-Butyl (R)-(4-((5-((1-(4-amino-7-(4-ethyl-5-oxo-2,5-dihydrofuran-3-yl) pyrrolo[2,1-f][1,2,4]triazin-5-yl)piperidin-3-yl)carbamoyl)-2-methylthiazol-4-yl)oxy)cyclohexyl)carbamate

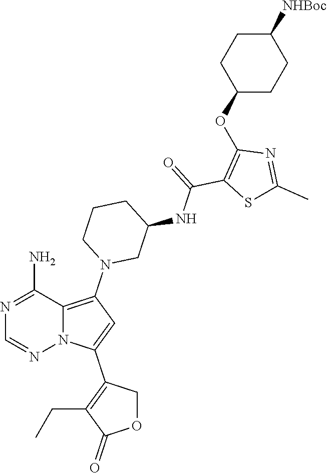

Analogous to the preparation of Example 996, reaction of Example 1002B (0.3 g, 0.84 mmol), Example 1018E (0.32 g, 0.84 mmol), BOP (0.41 g, 0.93 mmol) and DIPEA (0.59 mL, 3.37 mmol) in DMF (2 mL) at rt for 2 h resulted the crude which was washed with diethyl ether and dried to obtain Example 1018F (0.45 g, 79% yield); MS: [M+H]⁺=681.6

### Example 1018G. Preparation of N—((R)-1-(4-amino-7-(4-ethyl-5-oxo-2,5-dihydrofuran-3-yl) pyrrolo[2,1-f][1,2,4]triazin-5-yl)piperidin-3-yl)-4-(((1S,4S)-4-aminocyclohexyl)oxy)-2-methylthiazole-5-carboxamide. HCl

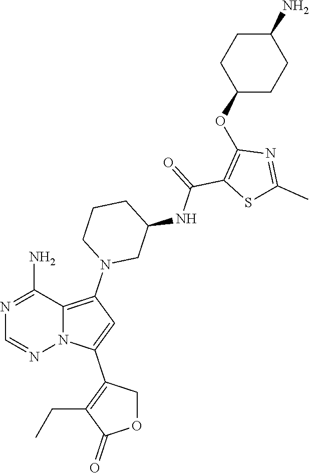 [SMILES: CCC1=C(c2cc(N3CCC[C@@H](NC(=O)c4sc(C)nc4O[C@H]4CC[C@@H](N)CC4)C3)c3c(N)ncnn23)COC1=O]

Analogous to the preparation of Example 996G, reaction of Example 1018F (1.75 g, 2.57 mmol) with 4M HCl in dioxane (3.21 mL, 12.85 mmol) in DCM (15 mL) are rt for 2 h afforded Example 1018G (1.3 g, 82% yield); MS: [M+H]⁺=581.5.

### Example 1018

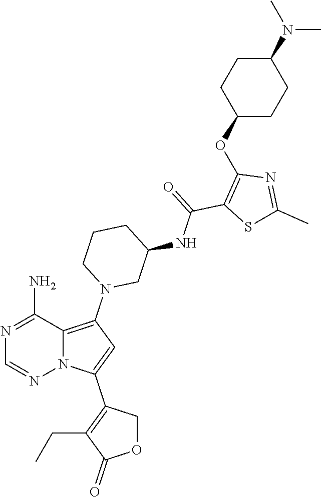 [SMILES: CCC1=C(c2cc(N3CCC[C@@H](NC(=O)c4sc(C)nc4O[C@H]4CC[C@@H](N(C)C)CC4)C3)c3c(N)ncnn23)COC1=O]

Analogous to the preparation of Example 996B, reaction of Example 1018G (0.07 g, 0.13 mmol), acetic acid (7.39 μl, 0.13 mmol), formaldehyde (0.02 mL, 0.90 mmol) and sodium cyanoborohydride (0.04 g, 0.64 mmol) in MeOH (2 mL) afforded crude which was purified via preparative LC/MS using Method L to afford Example 1018 (26.8 mg, 34% yield). LC-MS Method E: RT=1.50 min, MS: [M+H]⁺=609.3; LC-MS Method F: RT=1.14 min, MS: [M+H]⁺=609.3; ¹H NMR (400 MHz, DMSO-d₆) δ=8.35 (br s, 1H), 7.91 (s, 1H), 7.25 (br s, 1H), 6.99 (s, 1H), 6.89 (br s, 1H), 5.44 (s, 2H), 5.12 (br s, 1H), 4.20 (br s, 1H), 3.24 (br s, 2H), 2.97 (br s, 2H), 2.63-2.53 (m, 5H), 2.29-1.94 (m, 10H), 1.83 (br d, J=3.3 Hz, 2H), 1.73-1.43 (m, 7H), 1.10 (t, J=7.5 Hz, 3H).

Analogous to the preparation of Example 1018, Examples 1019 and 1020 were synthesized following chiral separation using method W (Table 70). Examples 1021, 1022, 1023, and 1024 were prepared by the acid amine coupling between Example 1018E and corresponding acids following the procedure described for the synthesis of Example 996.

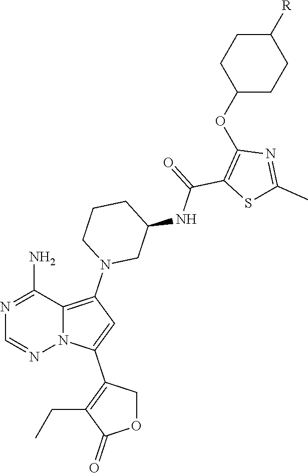

TABLE 70

| | | | | LCMS | |
|---|---|---|---|---|---|
| | Structure | | LCMS | Method, | |
| Example | (R) | Name | (M+H) + | RT (min.) | IHNMR |
| 1019 | OH | N-((R)-1-(4-amino-7-(4-ethyl-5- | 639.3 | B: 1.15 | 1H NMR (400 MHZ, DMSO-d6) § = 8.36 - |
| | SO-1 | oxo-2,5-dihydrofuran-3- | | | 8.21 (m, 1H), 7.93 - 7.88 (m, 1H), 7.38 - 7.19 |
| | | yl)pyrrolo[2,1-f][ 1,2,4]triazin-5- | | | (m, 1H), 7.05 - 6.83 (m, 2H), 5.49 - 5.48 (m, |
| | | y1)piperidin-3-y1)-4-(((1S, 4S)-4- | | | 1H), 5.46 - 5.42 (m, 2H), 5.15 - 5.10 (m, 1H), |
| | | ((1-hydroxypropan-2- | | | 4.22 - 4.10 (m, 2H), 3.22 - 3.12 (m, 3H), 3.07 |
| | | yl)amino)cyclohexyl)oxy)-2- | | | ~ 2.96 (m, 2H), 2.91 - 2.66 (m, 4H), 2.63 - |
| | | methylthiazole-5-carboxamide | | | 2.54 (m, 4H), 2.06 - 1.94 (m, 3H), 1.92 - 1.90 |
| | | (Method W) | | | (m, IH), 1.88 - 1.78 (m, IH), 1.77 - 1.61 (m, |
| | | | | | 3H), 1.49 - 1.38 (m, 2H), 1.37 - 1.28 (m, 2H), |
| | | | | | 1.11 (t, J = 7.5 Hz, 3H), 0.91 (br d, J = 6.0 |
| | | | | | Hz, 3H). |
| 1020 | OH | N-((R)-1-(4-amino-7-(4-ethyl-5- | 639.3 | B:1.15 | (400 MHz, DMSO- d6 ) 8 = 8.30 (br s, 1H), |
| | Iso-2 | oxo-2,5-dihydrofuran-3- | | | 7.91 (s, IH), 7.25 (br s, IH), 7.01 (s, 1H), |
| | | yl)pyrrolo[2,1-f][1,2,4]triazin-5- | | | 6.93 (br s, 1H), 5.45 (s, 2H), 5.08 (br s, 1H), |
| | | yl)piperidin-3-y1)-4-(((1S,4S)-4- | | | 4.16 (br s, 2H), 3.52 - 3.48 (m, 1H), 3.20 - |
| | | ((1-hydroxypropan-2- | | | 3.12 (m, 1H), 2.86 (br s, 6H), 2.64 - 2.54 (m, |
| | | yl)amino)cyclohexyl)oxy)-2- | | | 6H), 2.17 - 1.92 (m, 3H), 1.89 - 1.76 (m, 4H), |
| | | methylthiazole-5-carboxamide | | | 1.74 - 1.60 (m, 3H), 1.56 - 1.32 (m, 2H), 1.11 |
| | | (Method W) | | | (t, J = 7.5 Hz, 3H), 0.99 (br s, 3H). |
| Example | Structure | Name | LCMS | LCMS | IHNMR |
| | (R) | | (M+H) + | Method, | |
| | | | | RT (min.) | |
| 1021 | N | (R)-N-(1-(4-amino-7-(4-ethyl-5- | 637.2 | B: 1.13 | (400 MHz, DMSO-d6 ) ¿ = 8.40 (br s, IH), |
| | OH | oxo-2,5-dihydrofuran-3- | | | 7.91 (s, IH), 7.33 (br s, IH), 7.01 (s, IH), |
| | (Iso-2) | yl)pyrrolo[2, 1-f][ 1,2,4]triazin-5- | | | 6.86 (br s, IH), 5.45 (s, 2H), 5.30 (br s, 1H), |
| | | yl)piperidin-3-y1)-4-((4-(3- | | | 5.05 (br s, IH), 4.28- 4.01 (m, 2H), 3.69- 3.47 |
| | | hydroxyazetidin-1- | | | (m, 5H), 3.23 (br s, 2H), 2.95 (br s, 2H), 2.63 |
| | | yl)cyclohexyl)oxy)-2- | | | - 2.55 (m, 5H), 2.01 - 1.74 (m, 5H), 1.72 - |
| | | methylthiazole-5-carboxamide | | | 1.40 (m, 4H), 1.39 - 1.25 (m, 3H), 1.11 (t, J = |
| | | (Method M) | | | 7.4 Hz, 3H). |
| 1022 | INH | (R)-N-(1-(4-amino-7-(4-ethyl-5- | 621.3 | A:1.57 | (400 MHZ, DMSO- d6 ) o = 8.47 (s, 1H), 7.92 |
| | | oxo-2,5-dihydrofuran-3- | | | (s, IH), 7.32 (br s, IH), 7.02 (s, 1H), 6.89 (br |
| | | yl)pyrrolo[2,1-f][1,2,4]triazin-5- | | | s, IH), 5.44 (s, 2H), 5.05 (br s, 1H), 4.21 (br |
| | | yl)piperidin-3-y1)-4-((4- | | | s, IH), 3.24 (br s, 2H), 2.97 (br s, 5H), 2.63 - |
| | | (cyclopropylamino)cyclohexyl)o | | | 2.54 (m, 5H), 2.08 (s, IH), 1.96 - 1.73 (m, |
| | | xy)-2-methylthiazole-5- | | | 8H), 1.72 - 1.37 (m, 5H), 1.35 - 1.24 (m, 2H), |
| | | carboxamide | | | 1.10 (t, J = 7.4 Hz, 3H). |
| | | (Method N) | | | |
| Example | Structure | Name | LCMS | LCMS | 1HNMR |
| | (R) | | (M+H) + | Method, | |
| | | | | RT (min.) | |
| 1023 | NH | N-((R)-1-(4-amino-7-(4-ethyl-5- | 653.3 | A:1.70 | (400 MHZ, DMSO- d6 ) 8 = 8.28 (br s, 1H), |
| | (Iso-2) | oxo-2,5-dihydrofuran-3- | | B:1.23 | 7.91 (s, IH), 7.27 (br s, IH), 7.01 (s, 1H), |
| | | yl)pyrrolo[2, 1-f][1,2,4]triazin-5- | | | 6.89 (br s, IH), 5.55 - 5.37 (m, 2H), 5.13 (br |
| | | yl)piperidin-3-y1)-4-((4-(((S)-1- | | | s, IH), 4.19 (br s, IH), 3.22 - 3.07 (m, 6H), |
| | | methoxypropan-2- | | | 2.85 (br s, 6H), 2.64 - 2.53 (m, 4H), 2.05 - |
| | | yl)amino)cyclohexyl)oxy)-2- | | | 1.79 (m, 5H), 1.77 - 1.45 (m, 5H), 1.43 - 1.26 |
| | | methylthiazole-5-carboxamide | | | (m, 2H), 1.11 (t, J = 7.5 Hz, 3H), 0.89 (br s, |
| | | (Method N) | | | 3H). |
| 1024 | NH | N-((R)-1-(4-amino-7-(4-ethyl-5- | 653.3 | A:1.71 | (400 MHZ, DMSO- d6 ) 8 = 8.28 (br s, 1H), |
| | (Iso-2) | oxo-2,5-dihydrofuran-3- | | B:1.24 | 7.91 (s, 1H), 7.30 (br s, 1H), 7.00 (s, 1H), |
| | | yl)pyrrolo[2, 1-f][1,2,4]triazin-5- | | | 6.90 (br s, 1H), 5.44 (d, J = 3.3 Hz, 2H), 5.13 |
| | | yl)piperidin-3-y1)-4-((4-(((R)-1- | | | (br s, IH), 4.19 (br s, 1H), 3.28 - 3.22 (m, |
| | | methoxypropan-2- | | | 1H), 3.19 (s, 6H), 3.15 - 2.96 (m, 2H), 2.68 |
| | | yl)amino)cyclohexyl)oxy)-2- | | | (br s, 3H), 2.64 - 2.54 (m, 5H), 2.06 - 1.76 (m, |
| | | methylthiazole-5-carboxamide | | | 5H), 1.74 - 1.47 (m, 5H), 1.44 - 1.24 (m, 2H), |
| | | (Method N) | | | 1.11 (t, J = 7.5 Hz, 3H), 0.91 (br s, 3H). |

### Example 1025

### Preparation of N—((R)-1-(4-amino-7-(3-oxocyclopent-1-en-1-yl) pyrrolo[2,1-f] [1,2,4] triazin-5-yl) piperidin-3-yl)-4-(((1s,4S)-4-(dimethyl amino) cyclohexyl) oxy)-2-methylthiazole-5-carboxamide

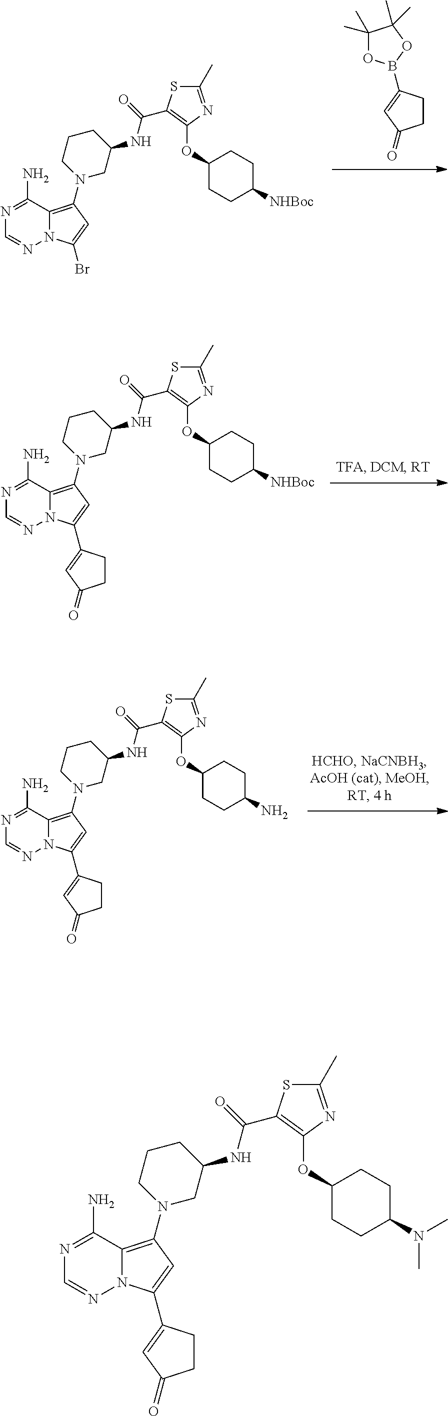

### Example 1025A. Preparation of tert-butyl ((1S,4s)-4-((5-(((R)-1-(4-amino-7-(3-oxocyclopent-1-en-1-yl) pyrrolo[2,1-f][1,2,4] triazin-5-yl) piperidin-3-yl) carbamoyl)-2-methylthiazol-4-yl) oxy) cyclohexyl) carbamate

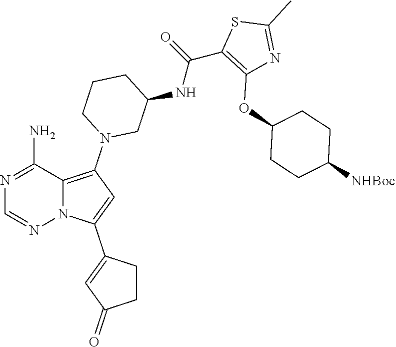 [SMILES: Cc1nc(O[C@H]2CC[C@@H](NC(=O)OC(C)(C)C)CC2)c(C(=O)N[C@@H]2CCCN(c3cc(C4=CC(=O)CC4)n4ncnc(N)c34)C2)s1]

Analogous to the preparation of Example 996F, reaction of Example 1002C (300 mg, 0.46 mmol), 3-(4,4,5,5-tetramethyl-1,3,2-dioxaborolan-2-yl) cyclopent-2-en-1-one (144 mg, 0.69 mmol), potassium phosphate tribasic (2M aq. solution in water) (0.57 mL, 1.15 mmol) and PdCl₂(dppf)·DCM complex (37.7 mg, 0.05 mmol) in THF (5 mL) at 70° C. for 6 h afforded the crude which was purified using Combi Flash instrument (24 g silica gel column; 2.3% MeOH in CHCl₃) to afford Example 1025A (240 mg, 51% yield) as an off white solid. [M+H]⁺=651.4.

### Example 1025B. Preparation of N—((R)-1-(4-amino-7-(3-oxocyclopent-1-en-1-yl) pyrrolo[2,1-f] [1,2,4] triazin-5-yl) piperidin-3-yl)-4-(((1S,4S)-4-aminocyclohexyl) oxy)-2-methylthiazole-5-carboxamide. TFA

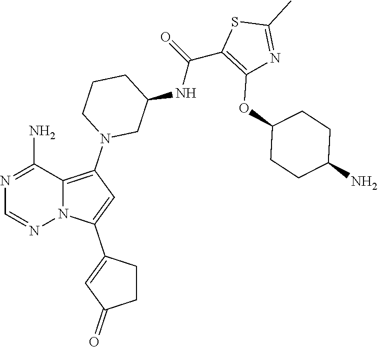 [SMILES: Cc1nc(O[C@H]2CC[C@@H](N)CC2)c(C(=O)N[C@@H]2CCCN(c3cc(C4=CC(=O)CC4)n4ncnc(N)c34)C2)s1]

Analogous to the preparation of Example 1002E, reaction of Example 1025A (240 mg, 0.36 mmol) with TFA (0.14 mL, 1.84 mmol) in DCM (5 mL) at rt for 16 h afforded the crude material which was triturated with diethyl ether (2×10 mL). The resulting solids were filtered, rinsed with n-pentane (10 mL), dried and collected to afford Example 1025B (200 mg, 67% yield) as an off-white solid. [M+H]⁺=551.4

### Example 1025

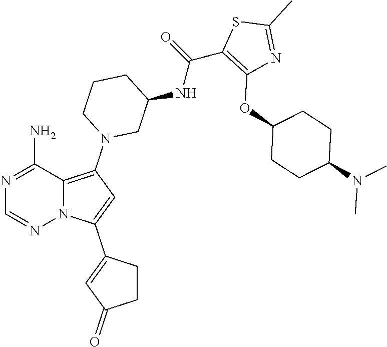 [SMILES: Cc1nc(O[C@H]2CC[C@@H](N(C)C)CC2)c(C(=O)N[C@@H]2CCCN(c3cc(C4=CC(=O)CC4)n4ncnc(N)c34)C2)s1]

Analogous to the preparation of Example 996B, reaction of Example 1025B (200 mg, 0.36 mmol), formaldehyde (37% in water) (0.13 mL, 1.81 mmol), AcOH (2.08 μl, 0.03 mmol) and sodium cyanoborohydride (45.6 mg, 0.72 mmol) in methanol (5 mL) at rt for 4 h afforded the crude material was purified via preparative LCMS/HPLC Method L to afford Example 1025 (31 mg, 14% yield) as an off-white solid. LC-MS Method E: RT=1.23 min, [M+H]⁺=579.3; LC-MS Method F: RT=0.95 min, [M+H]⁺=579.3; ¹H NMR (400 MHz, DMSO-d₆) δ=8.32 (br s, 1H), 8.02 (s, 1H), 7.25 (br s, 1H), 7.09 (s, 1H), 7.01 (t, J=1.5 Hz, 1H), 6.92 (br s, 1H), 5.11 (br s, 1H), 4.17 (br s, 1H), 3.24 (br s, 2H), 3.12-3.05 (m, 2H), 2.99 (br s, 1H), 2.83 (br s, 1H), 2.61 (s, 3H), 2.59-2.39 (m, J=2.6, 4.9 Hz, 9H), 2.17 (br s, 2H), 1.91 (s, 1H), 1.88-1.63 (m, 6H), 1.61-1.44 (m, 3H).

### Example 1026

### Preparation of (R)—N-(1-(4-amino-7-(5-(methylsulfonyl)pyridin-2-yl)pyrrolo[2,1-f][1,2,4]triazin-5-yl)piperidin-3-yl)-4-((4-(tert-butylamino)cyclohexyl)oxy)-2-methylthiazole-5-carboxamide

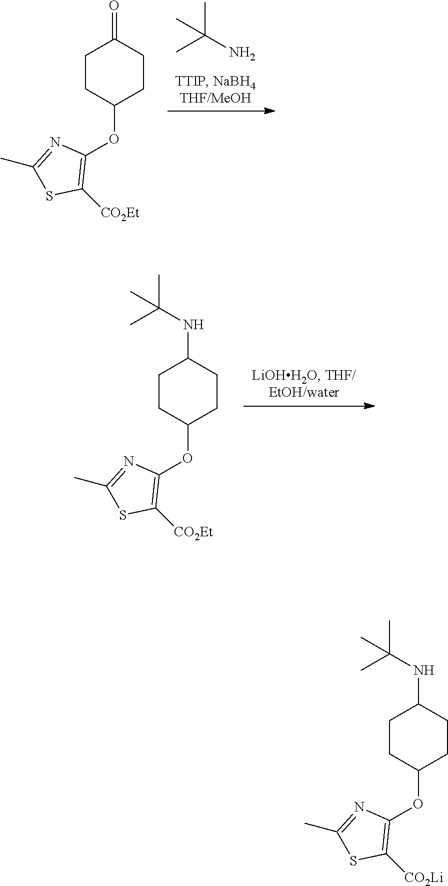

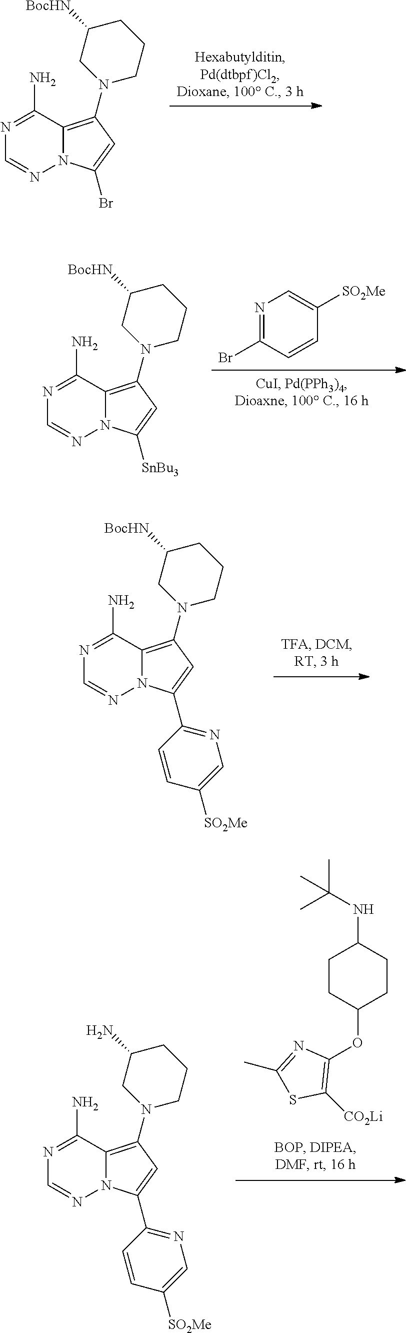

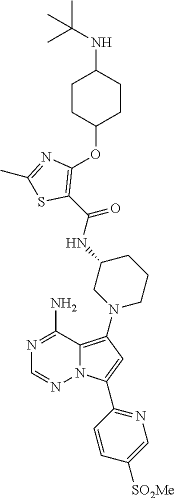 [SMILES: Cc1nc(OC2CCC(NC(C)(C)C)CC2)c(C(=O)N[C@@H]2CCCN(c3cc(-c4ccc(S(C)(=O)=O)cn4)n4ncnc(N)c34)C2)s1]

### Example 1026A. Preparation of ethyl 4-((4-(tert-butylamino)cyclohexyl)oxy)-2-methylthiazole-5-carboxylate

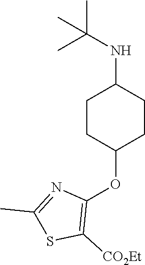 [SMILES: CCOC(=O)c1sc(C)nc1OC1CCC(NC(C)(C)C)CC1]

Analogous preparation of Example 1000A, reaction of Example 941B (500 mg, 1.76 mmol), 2-methylpropan-2-amine (323 mg, 4.41 mmol) titanium(IV) isopropoxide (0.52 mL, 1.76 mmol) and NaBH₄ (134 mg, 3.53 mmol) in THF (5 mL) afford crude yellow thick mass which was purified by silica gel chromatography (40 g Redisep® column, eluting with 0-20% MeOH in DCM) to afford the Example 1026A (50 mg, 8% yield) as yellow thick liquid. MS: [M+H]⁺=341.2.

### Example 1026B. Preparation of 4-((4-(tert-butylamino)cyclohexyl)oxy)-2-methylthiazole-5-carboxylic acid

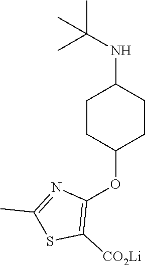

Analogous to the preparation of Example 996C, reaction of Example 1026A (50 mg, 0.14 mmol) and lithium hydroxide hydrate (18.49 mg, 0.44 mmol) in THF (2 mL)/ethanol (2 mL) afforded Example 1026B (40 mg, 87% yield) as off-white gummy solid. MS: [M+H]⁺=313.2.

### Example 1026C. Preparation of tert-butyl (R)-(1-(4-amino-7-(tributylstannyl)pyrrolo[2,1-f][1,2,4]triazin-5-yl)piperidin-3-yl)carbamate

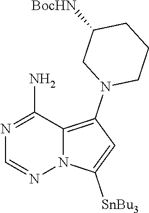

A stirred solution of tert-butyl (R)-(1-(4-amino-7-bromopyrrolo[2,1-f][1,2,4]triazin-5-yl)piperidin-3-yl)carbamate (2.0 g, 4.86 mmol) in dioxane (20 mL) was subjected to argon purging for 10 min. and then charged with hexabutylditin (6.14 mL, 12.16 mmol), and [1,1′-bis(di-tert butylphosphino)ferrocene]dichloropalladium(II) (0.31 g, 0.48 mmol). The mixture was stirred for 3 h at 100° C. The reaction mixture was cooled to rt and diluted with ethyl acetate (30 mL). The mixture was filtered through celite bed and washed with ethyl acetate (2×20 mL). The combined filtrate was concentrated, and the residue was purified by short column (40 g, 15 min. run 0-100%, compound eluted at 65-70%) to afford Example 1026C (1.1 g, 36% yield) as yellow gummy solid. MS: [M+H]⁺=621.20; ¹H NMR (400 MHz, DMSO-d₆) δ=7.67 (s, 2H), 6.99 (br s, 1H), 6.77-6.64 (m, 1H), 6.50 (s, 1H), 3.62 (br s, 2H), 3.30-3.18 (m, 1H), 3.03 (br d, J=8.0 Hz, 1H), 2.85 (br s, 1H), 2.74-2.55 (m, 2H), 1.79 (br s, 2H), 1.60-1.45 (m, 6H), 1.38 (s, 9H), 1.28 (sxt, J=7.3 Hz, 7H), 1.14-1.07 (m, 5H), 0.83 (t, J=7.3 Hz, 9H).

### Example 1026D. Preparation of tert-butyl (R)-(1-(4-amino-7-(5-(methylsulfonyl)pyridin-2-yl)pyrrolo[2,1-f][1,2,4]triazin-5-yl)piperidin-3-yl)carbamate

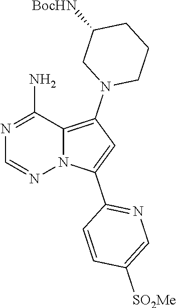 [SMILES: CC(C)(C)OC(=O)N[C@@H]1CCCN(c2cc(-c3ccc(S(C)(=O)=O)cn3)n3ncnc(N)c23)C1]

A suspension of Example 1026C (500 mg, 0.805 mmol) and 2-bromo-5-(methylsulfonyl)pyridine (209 mg, 0.88 mmol) in dioxane (5 mL) was purged with argon for 10 min. followed by the addition of copper(I) iodide (15.3 mg, 0.08 mmol) and Pd(Ph₃P)₄ (93 mg, 0.08 mmol). The mixture was stirred for 16 h at 100° C. After cooling to rt, the reaction mass was filtered through celite bed and then washed with ethyl acetate (30 mL). The filtrate was concentrated, and the residue was purified by silica gel chromatography (40 g Redisep® column, eluting with 0-100% EtOAc in hexane) to afford the Example 1026D (290 mg, 46% yield) as yellow solid. MS: [M+H]⁺=488.25.

### Example 1026E. Preparation of (R)-5-(3-aminopiperidin-1-yl)-7-(5-(methylsulfonyl)pyridin-2-yl)pyrrolo[2,1-f][1,2,4]triazin-4-amine. TFA

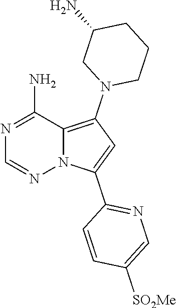 [SMILES: CS(=O)(=O)c1ccc(-c2cc(N3CCC[C@@H](N)C3)c3c(N)ncnn23)nc1]

Analogous to the preparation of Example 1002E, reaction of Example 1026D (290 mg, 0.59 mmol) and TFA (0.68 mL, 8.92 mmol) in DCM (3 mL) afforded Example 1026E (250 mg, 55% yield) as yellow solid. MS: [M+H]⁺=388.15; ¹H NMR (400 MHz, CHLOROFORM-d) δ=9.21 (s, 1H), 8.77 (d, J=8.0 Hz, 1H), 8.70-8.50 (m, 2H), 8.39-8.27 (m, 1H), 7.86 (s, 1H), 7.64 (s, 1H), 3.86-3.68 (m, 1H), 3.52 (d, J=7.0 Hz, 1H), 3.39-3.30 (m, 1H), 3.19 (s, 3H), 2.98-2.88 (m, 1H), 2.68-2.50 (m, 1H), 2.26-2.14 (m, 2H), 1.46 (s, 1H), 1.24 (t, J=7.0 Hz, 2H), 0.95-0.72 (m, 1H).

### Example 1026

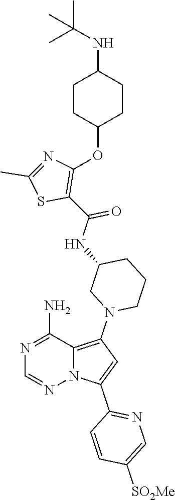 [SMILES: Cc1nc(OC2CCC(NC(C)(C)C)CC2)c(C(=O)N[C@@H]2CCCN(c3cc(-c4ccc(S(C)(=O)=O)cn4)n4ncnc(N)c34)C2)s1]

Analogous to the preparation of Example 996, reaction of Example 1026E (80 mg, 0.16 mmol), Example 1026B (50 mg, 0.16 mmol), BOP (85 mg, 0.19 mmol) and DIPEA (0.08 mL, 0.48 mmol) in DMF (2 mL) afforded Example 1026 (20.5 mg, 18% yield) as pale-yellow solid. LC-MS Method E: RT=1.36 min, [M+H]⁺=682.2; LC-MS Method F: RT=1.10 min, [M+H]⁺=682.2; ¹H NMR (400 MHz, DMSO-d₆) δ ppm 9.03 (d, J=0.8 Hz, 1H), 8.93-8.85 (m, 1H), 8.39 (dd, J=2.5, 8.5 Hz, 1H), 8.02 (s, 1H), 7.42 (s, 1H), 7.35-7.14 (m, 1H), 5.17-5.03 (m, 1H), 4.24-4.13 (m, 1H), 3.06-2.86 (m, 4H), 2.60 (s, 3H), 2.08-1.95 (m, 3H), 1.89-1.80 (m, 2H), 1.80-1.42 (m, 10H), 1.10-0.98 (m, 9H), 0.93-0.85 (m, 3H).

### Example 1027

### Preparation of N—((R)-1-(4-amino-7-(4-(methylsulfonyl) phenyl) pyrrolo[2,1-f][1,2,4] triazin-5-yl) piperidin-3-yl)-4-((4-(3-hydroxycyclobutyl) cyclohexyl) oxy)-2-methylthiazole-5-carboxamide

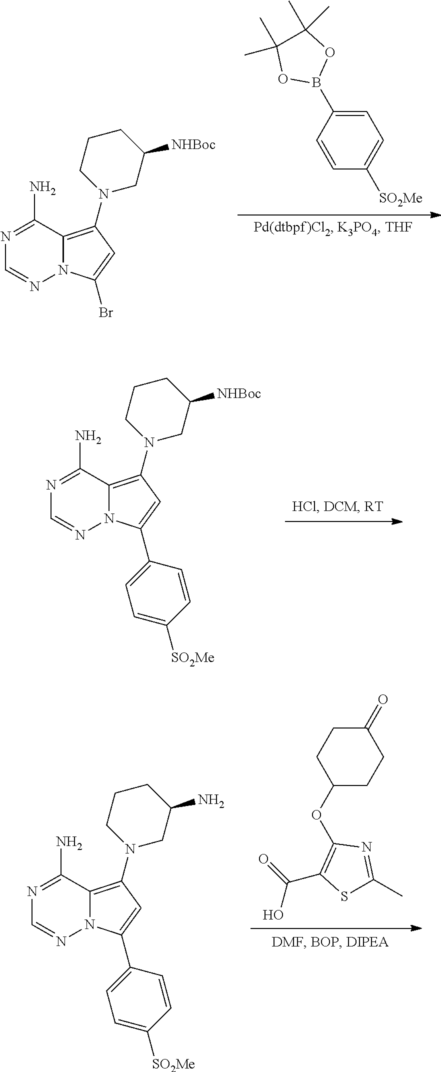

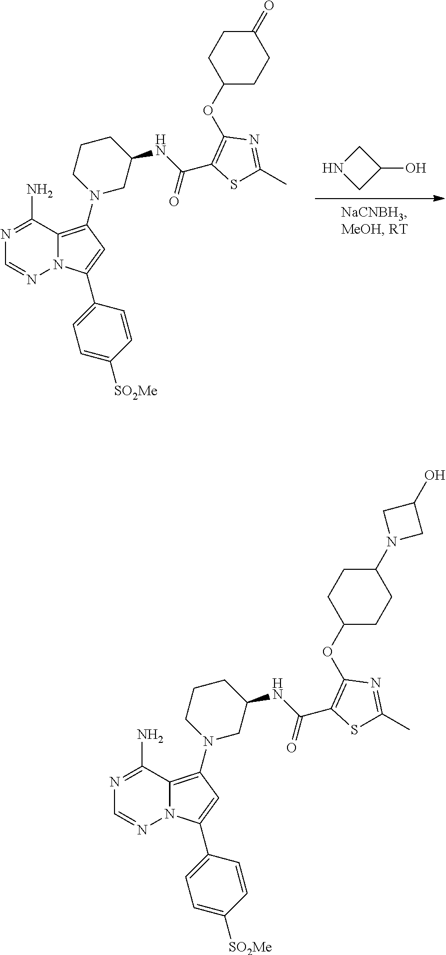

### Example 1027A: Preparation of tert-butyl (R)-(1-(4-amino-7-(4-(methylsulfonyl) phenyl) pyrrolo[2,1-f][1,2,4] triazin-5-yl) piperidin-3-yl) carbamate

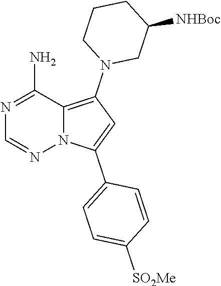 [SMILES: CC(C)(C)OC(=O)N[C@@H]1CCCN(c2cc(-c3ccc(S(C)(=O)=O)cc3)n3ncnc(N)c23)C1]

Alternatively, analogous to the preparation of Example 996F, reaction of tert-butyl (1R)-(1-(4-amino-7-bromopyrrolo[2,1-f] [1,2,4] triazin-5-yl) piperidin-3-yl) carbamate (1.5 g, 3.65 mmol), (4-methanesulfonylphenyl) boronic acid (1.09 g, 5.47 mmol), potassium phosphate, tribasic, (1.54 g, 7.29 mmol) and [1,1′-bis(di-tert-butylphosphino) ferrocene] dichloropalladium(II) (0.24 g, 0.36 mmol) in THF (10 mL)/water (0.2 mL) at 70° C. for 4 h afforded Example 1027A (950 mg, 87% yield). MS: [M+H]⁺=685.6.

### Example 1027B: Preparation of (R)-5-(3-aminopiperidin-1-yl)-7-(4-(methylsulfonyl) phenyl) pyrrolo[2,1-f][1,2,4]triazin-4-amine. HCl

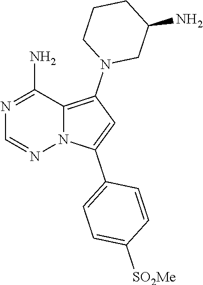 [SMILES: CS(=O)(=O)c1ccc(-c2cc(N3CCC[C@@H](N)C3)c3c(N)ncnn23)cc1]

Analogous to the preparation of Example 996G, reaction of Example 1027A (1.5 g, 3.08 mmol) with HCl in 1,4 dioxane (0.67 g, 18.50 mmol) in DCM (15 mL) at rt for 4 h afforded Example 1027B (1 g, 84% yield) as a yellow solid. MS: [M+H]⁺387.3.

### Example 1027C: Preparation of (R)—N-(1-(4-amino-7-(4-(methylsulfonyl) phenyl) pyrrolo[2,1-f][1,2,4]triazin-5-yl) piperidin-3-yl)-2-methyl-4-((4-oxocyclohexyl) oxy) thiazole-5-carboxamide

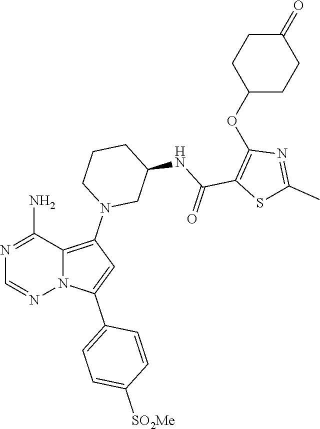 [SMILES: Cc1nc(OC2CCC(=O)CC2)c(C(=O)N[C@@H]2CCCN(c3cc(-c4ccc(S(C)(=O)=O)cc4)n4ncnc(N)c34)C2)s1]

Analogous to the preparation of Example 996, reaction of Example 1027B (Example 13A) (500 mg, 1.29 mmol), Example 1011A (495 mg, 1.94 mmol), DIPEA (0.90 mL, 5.17 mmol) and BOP (858 mg, 1.94 mmol) in DMF (0.5 mL) at it for 2 h afforded crude Example 1027C (350 mg, 0.56 mmol, 43% yield). MS: [M+H]⁺=624.2; ¹H NMR (400 MHz, DMSO-d₆) δ ppm 7.87 (d, J=1.9 Hz, 2H), 7.33 (br s, 1H), 6.85-6.61 (m, 3H), 5.13-4.95 (m, 1H), 4.69 (br d, J=1.8 Hz, 1H), 4.48 (br d, J=8.1 Hz, 2H), 4.27 (br d, J=2.3 Hz, 1H), 4.21-4.04 (m, 1H), 2.90 (br s, 4H), 2.61 (s, 3H), 2.20-1.98 (m, 5H), 1.95-1.72 (m, 2H),), 1.21-0.79 (m, 8H).

### Example 1027

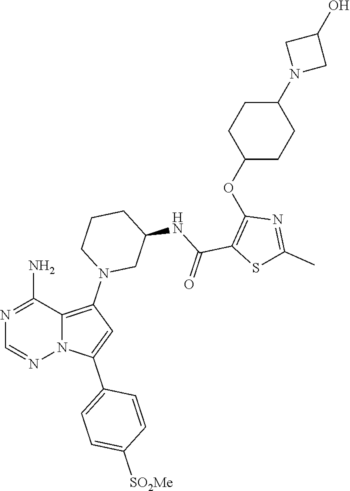 [SMILES: Cc1nc(OC2CCC(N3CC(O)C3)CC2)c(C(=O)N[C@@H]2CCCN(c3cc(-c4ccc(S(C)(=O)=O)cc4)n4ncnc(N)c34)C2)s1]

Analogous to the preparation of Example 996B, reaction of Example 1027C (125 mg, 0.20 mmol), azetidin-3-ol (29.3 mg, 0.40 mmol) and sodium cyanoborohydride (76 mg, 1.20 mmol) in MeOH (2 mL) at rt for 4 h afforded the crude which was purified by preparative HPLC to yield two isomers. Iso-2 (Example 1027); LC-MS Method F: RT=1.00 min [M+H]⁺=681.2; ¹H NMR (400 MHz, DMSO-d₆) δ=8.36 (d, J=8.5 Hz, 3H), 7.97 (d, J=8.5 Hz, 2H), 7.92 (s, 1H), 7.32 (br s, 1H), 7.21 (s, 1H), 6.84 (br s, 1H), 5.27 (br s, 1H), 5.06 (br s, 1H), 4.20 (br s, 1H), 4.14-4.04 (m, 1H), 3.54 (br s, 1H), 3.24 (s, 3H), 3.06-2.64 (m, 3H), 2.60 (s, 3H), 2.18 (br s, 3H), 1.96-1.77 (m, 5H), 1.72-1.43 (m, 4H), 1.41-1.26 (m, 9H).

### Example 1028

### Preparation of N—((R)-1-(4-amino-7-(2-methoxypyrimidin-4-yl)pyrrolo[2,1-f][1,2,4]triazin-5-yl)piperidin-3-yl)-4-(((1S,4S)-4-(dimethylamino)cyclohexyl)oxy)-2-methylthiazole-5-carboxamide

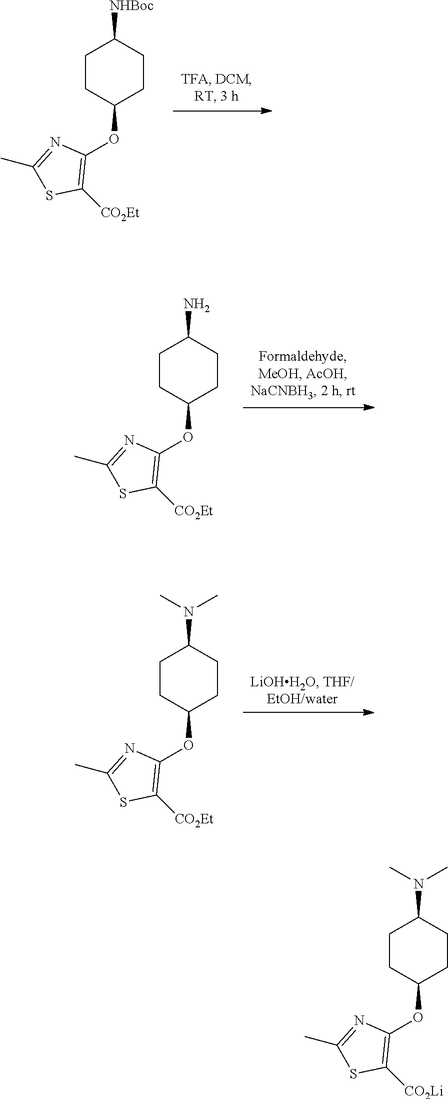

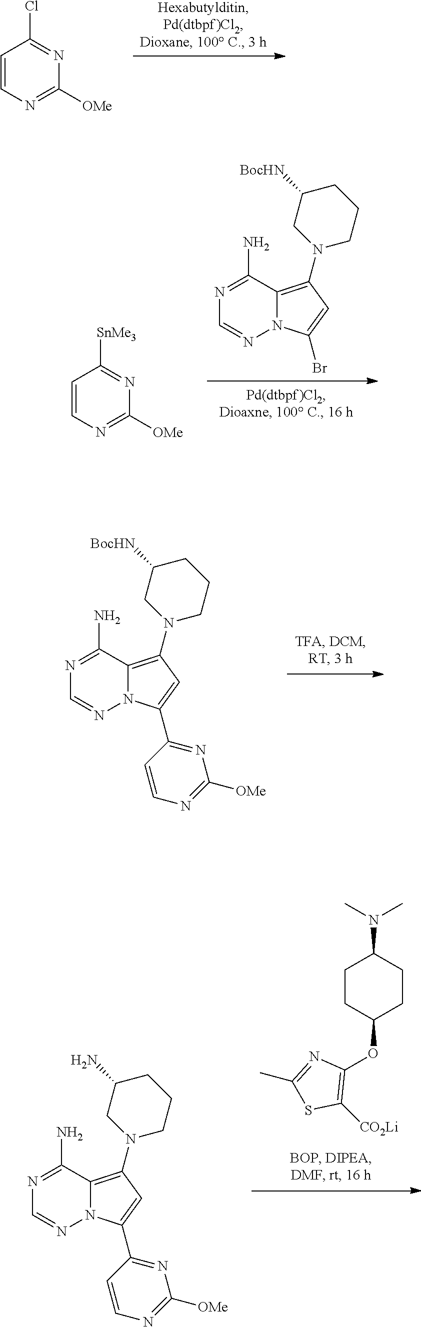

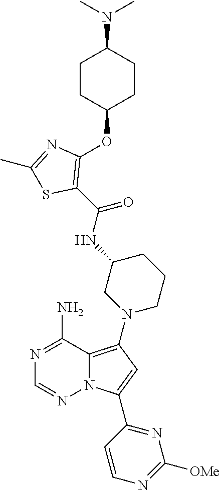 [SMILES: COc1nccc(-c2cc(N3CCC[C@@H](NC(=O)c4sc(C)nc4O[C@H]4CC[C@@H](N(C)C)CC4)C3)c3c(N)ncnn23)n1]

### Example 1028A. Preparation of ethyl 4-(((1S,4S)-4-aminocyclohexyl)oxy)-2-methylthiazole-5-carboxylate. TFA

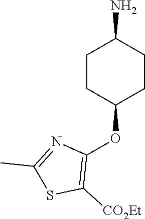 [SMILES: CCOC(=O)c1sc(C)nc1O[C@H]1CC[C@@H](N)CC1]

Analogous to the preparation of Example 1002E, reaction of Example 1002A (3.0 g, 7.80 mmol) and TFA (9.02 mL, 117 mmol) in DCM (30 mL) afforded Example 1028A (2.5 g, 76% yield) as yellow solid. MS: [M+H]⁺=285.15; ¹H NMR (400 MHz, DMSO-d₆) δ=5.08 (br s, 1H), 4.77 (td, J=6.3, 12.5 Hz, 3H), 4.23-4.15 (m, 2H), 3.39 (q, J=7.0 Hz, 1H), 2.63-2.57 (m, 3H), 2.01 (br d, J=9.0 Hz, 2H), 1.81-1.65 (m, 5H), 1.29-1.24 (m, 3H).

### Example 1028B. Preparation of ethyl 4-(((1S,4S)-4-(dimethylamino)cyclohexyl)oxy)-2-methylthiazole-5-carboxylate

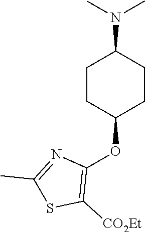

Analogous to the preparation of Example 99B, reaction of Example 1028A (2.5 g, 6.28 mmol), acetic acid (0.35 mL, 6.28 mmol), sodium cyanoborohydride (0.78 g, 12.55 mmol) and formaldehyde (1.53 g, 18.83 mmol) in methanol (25 mL) afforded the crude which was purified by silica gel chromatography (40 g Redisep® column, eluting with 0-20% MeOH in DCM) to obtain the Example 1028B (450 mg, 23% yield) as yellow thick liquid. MS: [M+H]⁺=313.10; ¹H NMR (400 MHz, CHLOROFORM-d) δ=5.31 (br d, J=10.5 Hz, 1H), 4.31-4.24 (m, 2H), 3.35-3.26 (m, 1H), 2.89 (s, 6H), 2.65 (s, 3H), 2.32-2.11 (m, 4H), 1.93-1.86 (m, 2H), 1.75-1.72 (m, 2H), 1.46-1.24 (m, 3H).

### Example 1028C. Preparation of 4-(((1S,4S)-4-(dimethylamino)cyclohexyl)oxy)-2-methylthiazole-5-carboxylic acid

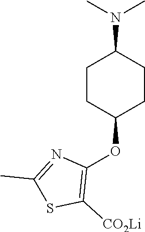

Analogous to the preparation of Example 996C, reaction of Example 1028B (450 mg, 1.44 mmol) and lithium hydroxide hydrate (151 mg, 3.60 mmol) in THF (2 mL)/ethanol (2 mL)/water (0.5 mL) afforded Example 1028C (350 mg, 81% yield) as off white solid. MS: [M+H]⁺=285.10; ¹H NMR (400 MHz, DMSO-d₆) δ=7.25 (s, 1H), 5.11-5.00 (m, 1H), 2.70-2.58 (m, 9H), 2.35-2.27 (m, 1H), 2.01 (br d, J=13.0 Hz, 2H), 1.93-1.74 (m, 3H), 1.63 (br s, 2H).

### Example 1028D. Preparation of 2-methoxy-4-(trimethylstannyl)pyrimidine

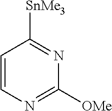

Analogous to the preparation of Example 1028C, reaction of 4-chloro-2-methoxypyrimidine (1.0 g, 6.92 mmol), hexamethylditin (1.57 mL, 7.61 mmol) and [1,1′-bis(di-tert-butylphosphino)ferrocene]dichloropalladium(II)(0.45 g, 0.69 mmol) in dioxane (5 mL) to afforded Example 1028D (1.0 g, 3.66 mmol, 53% yield) as brown gummy solid. MS: [M+H]⁺=275.0.

### Example 1028E. Preparation of tert-butyl (R)-(1-(4-amino-7-(2-methoxypyrimidin-4-yl)pyrrolo[2,1-f][1,2,4]triazin-5-yl)piperidin-3-yl)carbamate

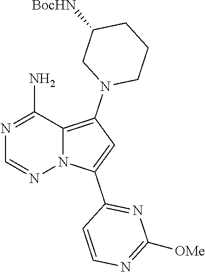 [SMILES: COc1nccc(-c2cc(N3CCC[C@@H](NC(=O)OC(C)(C)C)C3)c3c(N)ncnn23)n1]

To a stirred solution of tert-butyl (R)-(1-(4-amino-7-bromopyrrolo[2,1-f][1,2,4]triazin-5-yl)piperidin-3-yl)carbamate (2.2 g, 5.35 mmol) in dioxane (30 mL) was added Example 1028D (1.9 g, 6.95 mmol) and then purged with argon for 10 min. To this mixture was added [1,1′-bis(di-tert butylphosphino)ferrocene]dichloropalladium(II) (0.35 g, 0.53 mmol) and stirred for 16 h at 100° C. Reaction was cooled to rt, filtered through celite bed and the celite bed was washed with dioxane (30 mL). The filtrate was concentrated to afford brown gummy solid, which was purified by silica gel chromatography (120 g Redisep® column, eluting with 0-100% EtOAc in DCM) to afford the Example 1028E (2.3 g, 98% yield) as brown solid. MS: [M+H]⁺=441.2; ¹H NMR (300 MHz, DMSO-d₆) δ=8.64 (d, J=5.0 Hz, 1H), 8.20 (br s, 1H), 7.69 (s, 1H), 7.46 (d, J=2.9 Hz, 1H), 7.17-6.95 (m, 2H), 6.53 (d, J=2.9 Hz, 1H), 4.03 (q, J=7.2 Hz, 3H), 3.97 (s, 3H), 3.73-3.59 (m, 2H), 3.10-2.98 (m, 2H), 1.86-1.66 (m, 3H), 1.38 (s, 9H).

### Example 1028F. Preparation of (R)-5-(3-aminopiperidin-1-yl)-7-(2-methoxypyrimidin-4-yl)pyrrolo[2,1-f][1,2,4]triazin-4-amine. TFA

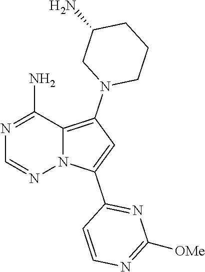 [SMILES: COc1nccc(-c2cc(N3CCC[C@@H](N)C3)c3c(N)ncnn23)n1]

Analogous to the preparation of Example 1002E, reaction of Example 1028E (2.3 g, 5.22 mmol) and TFA (6.03 mL, 78 mmol) in DCM (25 mL) afforded Example 1028F (2.0 g. 84% yield) as yellow solid. MS: [M+H]⁺=341.10; ¹H NMR (400 MHz, DMSO-d₆) δ=8.48 (br s, 1H), 7.41 (s, 1H), 7.28 (br s, 1H), 6.73 (br s, 1H), 3.98 (s, 3H), 3.39 (q, J=7.0 Hz, 1H), 3.29-3.15 (m, 2H), 2.93 (br s, 2H), 2.00-1.82 (m, 3H), 1.72 (br s, 4H), 1.09 (br t, J=7.0 Hz, 1H).

### Example 1028

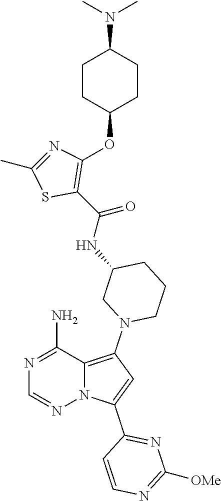 [SMILES: COc1nccc(-c2cc(N3CCC[C@@H](NC(=O)c4sc(C)nc4O[C@H]4CC[C@@H](N(C)C)CC4)C3)c3c(N)ncnn23)n1]

Analogous to the preparation or Example 99, reaction of Example 1028F (313 mg, 0.69 mmol), Example 1028C (200 mg, 0.69 mmol), BOP (366 mg, 0.82 mmol) and DIPEA (0.36 mL, 2.07 mmol) in DMF (5 mL) at rt for 16 h afforded the crude which was purified by reverse phase purification to afford Example 1028 (85 mg, 20% yield) as off white solid. MS. [M+H]⁺=607.30; HPLC Method E: RT=6.79 min, Method J: RT=8.29 min, ¹H NMR (400 MHz, DMSO-d₆) δ=8.64 (d, J=5.3 Hz, 1H), 8.31 (d, J=5.3 Hz, 2H), 8.02 (s, 1H), 7.39 (s, 1H), 7.24 (br s, 1H), 6.97 (br s, 1H), 5.12 (br s, 1H), 4.20 (br s, 1H), 3.96 (s, 3H), 3.27-3.13 (m, 1H), 2.98 (br s, 1H), 2.82 (br s, 1H), 2.62-2.53 (m, 4H), 2.33 (br s, 1H), 2.07 (br s, 6H), 1.98 (br d, J=14.1 Hz, 3H), 1.83 (br s, 2H), 1.63 (br t, J=12.3 Hz, 4H), 1.55-1.32 (m, 3H).

### Example 1029

### Preparation of N—((R)-1-(4-amino-7-(2-methoxypyrimidin-4-yl)pyrrolo[2,1-f][1,2,4]triazin-5-yl)piperidin-3-yl)-2-methyl-4-(((1s,4S)-4-(pyrrolidin-1-yl)cyclohexyl)oxy)thiazole-S-carboxamidemorpholinothiophene-2-carboxamide

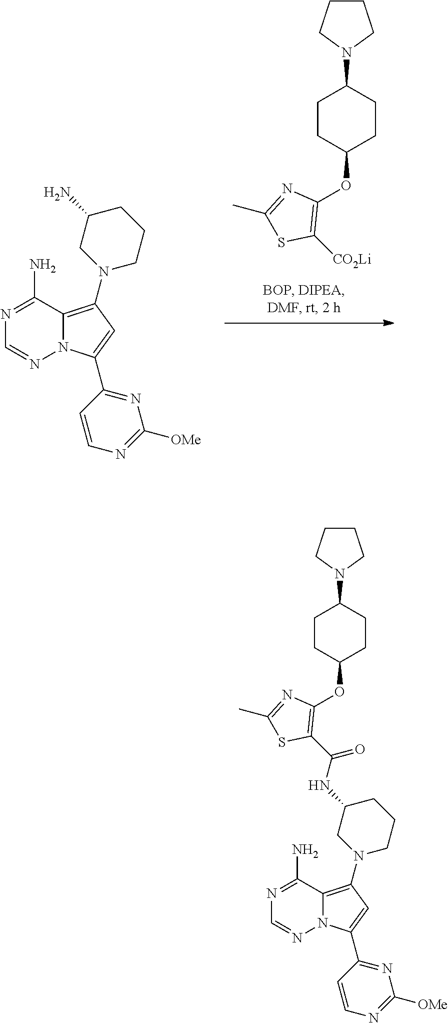

Analogous to the preparation of Example 996, reaction of Example 1028F (108 mg, 0.32 mmol), lithium 2-methyl-4-(((1S,4S)-4-(pyrrolidin-1-yl)cyclohexyl)oxy)thiazole-5-carboxylate (100 mg, 0.32 mmol). DIPEA (0.07 mL, 0.41 mmol) and BOP (168 mg, 0.38 mmol) in DMF (3 mL) at rt for 2 h afforded the crude which was purified via reverse phase chromatography to afford Example 1029 (27.3 mg, 14% yield) as pale yellow solid. LC-MS Method E: RT=1.45 min, [M+H]⁺=633.3; LC-MS Method F: RT=1.11 min, [M+H]⁺=633.3; ¹H NMR (400 MHz, DMSO-d₆) δ=8.64 (d, J=53 Hz, 1H), 8.32 (d, J=5.3 Hz, 2H), 8.02 (s, 1H), 7.38 (s, 1H), 7.04-6.75 (m, 1H), 5.11 (br s, 1H), 4.20 (br s, 1H), 3.97 (s, 3H), 3.24-3.09 (m, 1H), 2.96 (br s, 2H), 2.82-2.64 (m, 1H), 2.61 (s, 4H), 2.57-2.53 (m, 1H), 2.39 (br s, 1H), 2.36-2.21 (m, 1H), 1.97 (br s, 2H), 1.91 (s, 2H), 1.84 (br s, 3H), 1.74-1.55 (m, 5H), 1.50 (br s, 6H).

### Example 1030

### Preparation of (R)—N-(1-(4-amino-7-(2-methoxypyrimidin-4-yl)pyrrolo[2,1-f][1,2,4]triazin-5-yl)piperidin-3-yl)-2-methyl-4-((4-(tert-pentylamino)cyclohexyl)oxy)thiazole-5-carboxamide

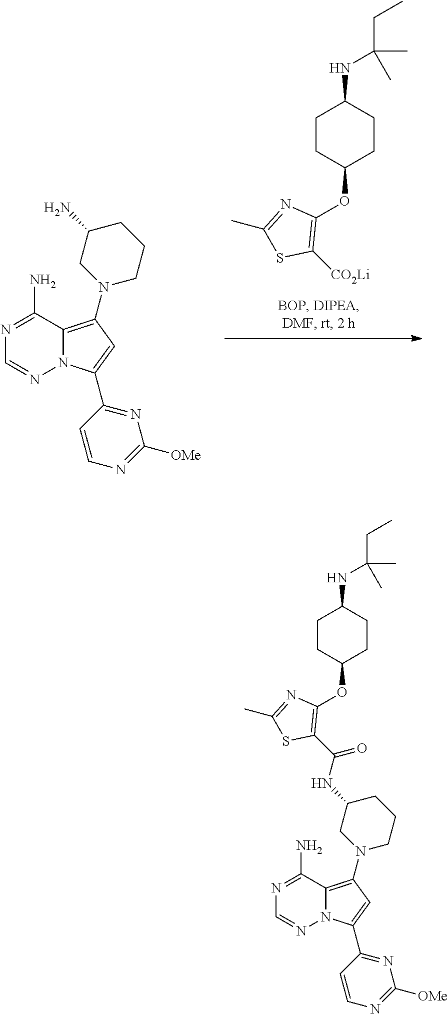

Analogous to the preparation of Example 996, reaction of Example 1028F (113 mg, 0.30 mmol), lithium 2-methyl-4-((4-(tert-pentylamino)cyclohexyl)oxy)thiazole-5-carboxylate (100 mg, 0.30 mmol). BOP (160 mg, 0.36 mmol) and DIPEA (0.15 mL, 0.90 mmol) in DMF (3 mL) at rt for 2 h afforded the crude which was purified via reverse phase chromatography to afford Example 1030 (Iso-1)(17.2 mg, 9% yield) as pale yellow solid. LC-MS Method E: RT=1.63 min, [M+H]⁺=649.3; LC-MS Method F: RT=1.21 min, [M+H]⁺=649.3; ¹H NMR (400 MHz, DMSO-d₆) δ=8.64 (d, J=5.3 Hz, 1H), 8.32 (d, J=5.5 Hz, 2H), 8.02 (s, 1H), 7.41 (s, 1H), 7.38-7.27 (m, 1H), 5.12 (br s, 1H), 4.21 (br s, 1H), 3.96 (s, 3H), 3.19-3.11 (m, 1H), 2.96 (br s, 3H), 2.60 (s, 3H), 2.57-2.53 (m, 1H), 1.98-1.76 (m, 6H), 1.75-1.54 (m, 3H), 1.51 (br s, 1H), 1.39-1.18 (m, 3H), 1.13 (br s, 2H), 0.86 (br s, 1H), 0.78 (br s, 5H), 0.60 (br s, 4H).

### Example 1031

### Preparation of (R)—N-(1-(4-amino-7-(2-methoxypyrimidin-4-yl)pyrrolo[2,1-f][1,2,4]triazin-5-yl)piperidin-3-yl)-4-((4-(bis(methyl-d3)amino)cyclohexyl)oxy)-2-methylthiazole-5-carboxamide

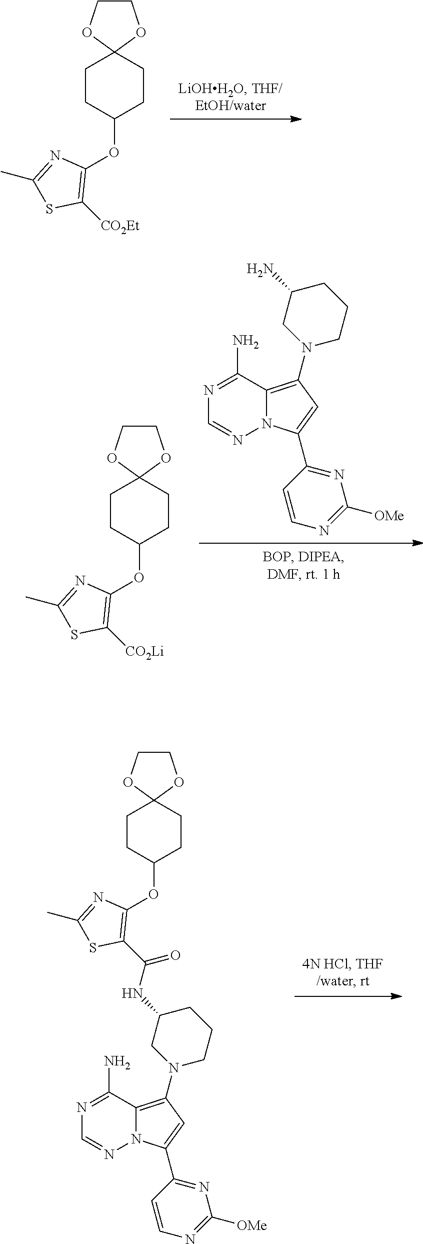

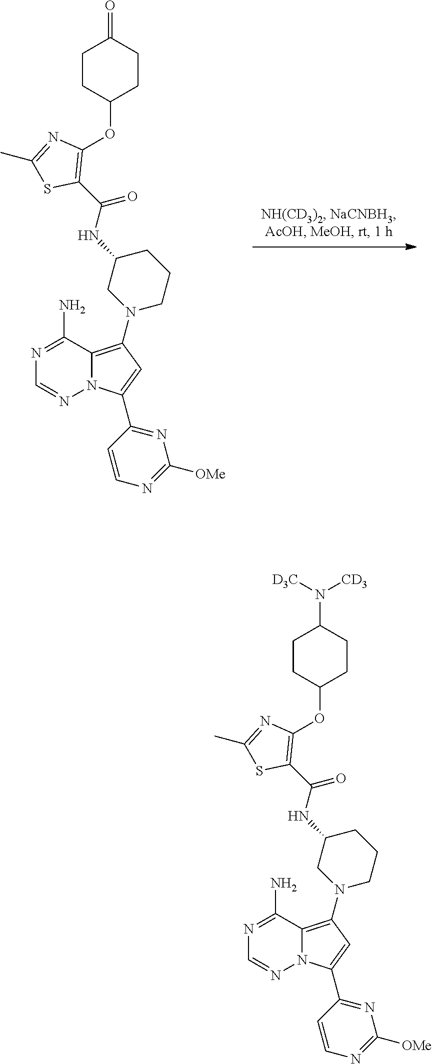 [SMILES: COc1nccc(-c2cc(N3CCC[C@@H](NC(=O)c4sc(C)nc4OC4CCC(=O)CC4)C3)c3c(N)ncnn23)n1 ; "NH(CD3)2, NaCNBH3,AcOH, MeOH, rt, 1 h" ; [2H]C([2H])([2H])N(C1CCC(Oc2nc(C)sc2C(=O)N[C@@H]2CCCN(c3cc(-c4ccnc(OC)n4)n4ncnc(N)c34)C2)CC1)C([2H])([2H])[2H]]

### Example 1031A. Preparation of lithium 4-((1,4-dioxaspiro[4.5]decan-8-yl)oxy)-2-methylthiazole-5-carboxylate

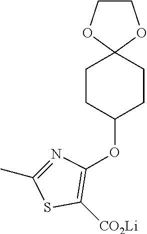

Analogous to the preparation of Example 996C, reaction of Example 996A (5 g, 15.3 mmol) and LiOH·H₂O (1.09 g, 45.8 mmol) in ethanol (20 mL)/THF (30 mL)/water (10 mL) afforded Example 1031A (4.2 g, 90% yield) as white solid. MS: [M+H]⁺=300.05; ¹H NMR (400 MHz, CHLOROFORM-d) δ=5.22-5.16 (m, 1H), 3.99 (br s, 4H), 2.67 (s, 3H), 2.12-1.85 (m, 6H), 1.71 (ddd, J=5.0, 8.1, 12.9 Hz, 2H).

### Example 1031B. Preparation of (R)-4-((1,4-dioxaspiro[4.5]decan-8-yl)oxy)-N-(1-(4-amino-7-(2-methoxypyrimidin-4-yl)pyrrolo[2,1-f][1,2,4]triazin-5-yl)piperidin-3-yl)-2-methylthiazole-5-carboxamide

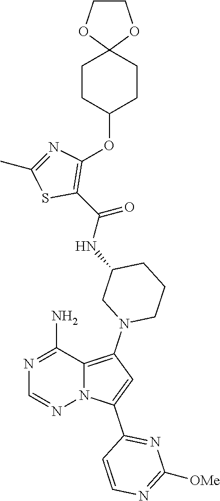 [SMILES: COc1nccc(-c2cc(N3CCC[C@@H](NC(=O)c4sc(C)nc4OC4CCC5(CC4)OCCO5)C3)c3c(N)ncnn23)n1]

Analogous to the preparation of Example 996, reaction of Example 1028F (222 mg, 0.59 mmol) and Example 1031A (180 mg, 0.59 mmol), BOP (286 mg, 0.65 mmol) and DIPEA (0.2 mL, 1.2 mmol) in DMF (5 mL) afforded the crude which was purified through silica gel column to get Example 1031B (170 mg, 46% yield) as yellow solid. MS: [M+H]⁺=622.25. ¹H NMR (400 MHz, DMSO-d₆) δ=8.64 (d, J=5.5 Hz, 1H), 8.32 (d, J=5.0 Hz, 1H), 8.02 (s, 1H), 7.43 (s, 1H), 7.24 (br s, 1H), 6.97 (br s, 1H), 5.10 (br s, 1H), 4.15 (br s, 1H), 3.97 (s, 2H), 3.85 (br d, J 8.5 Hz, 4H), 3.58-3.36 (m, 2H), 3.00 (br s, 1H), 2.85 (br s, 1H), 2.69-2.53 (m, 4H), 1.97-1.79 (m, 7H), 1.79-1.67 (m, 2H), 1.60 (br s, 3H).

### Example 1031C. Preparation of (R)—N-(1-(4-amino-7-(2-methoxypyrimidin-4-yl)pyrrolo[2,1-f][1,2,4]triazin-5-yl)piperidin-3-yl)-2-methyl-4-((4-oxocyclohexyl)oxy)thiazole-5-carboxamide. HCl

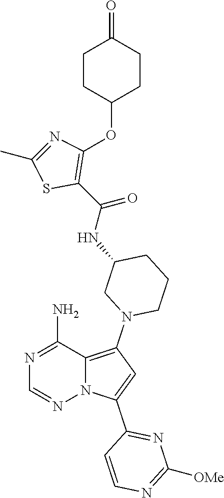 [SMILES: COc1nccc(-c2cc(N3CCC[C@@H](NC(=O)c4sc(C)nc4OC4CCC(=O)CC4)C3)c3c(N)ncnn23)n1]

Analogous to the preparation of Example 941B, reaction of Example 1031B (170 mg, 0.27 mmol) and HCl (4M in dioxane) (1.02 mL, 4.10 mmol) in THF (1 mL)/water (1 mL) afforded Example 1031C (150 mg, 95% yield) as yellow solid. MS: [M+H]⁺=659.5; ¹H NMR (400 MHz, DMSO-d₆) δ=8.61 (d, J=5.5 Hz, 1H), 8.28 (d, J=5.5 Hz, 1H), 7.97 (s, 1H), 7.39 (s, 1H), 5.30 (br s, 1H), 4.11 (br d, J=3.5 Hz, 1H), 3.55 (s, 3H), 3.17-2.75 (m, 3H), 2.63-2.53 (m, 3H), 2.49-2.27 (m, 5H), 2.23-2.06 (m, 4H), 1.97 (s, 1H), 1.78 (br s, 3H), 1.53 (br s, 1H).

### Example 1031 and Example 1032

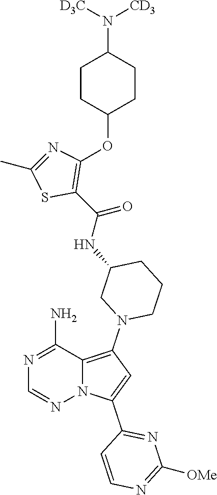 [SMILES: [2H]C([2H])([2H])N(C1CCC(Oc2nc(C)sc2C(=O)N[C@@H]2CCCN(c3cc(-c4ccnc(OC)n4)n4ncnc(N)c34)C2)CC1)C([2H])([2H])[2H]]

Analogous to the preparation of Example 996B, reaction of bis(methyl-d3)amine.HCl (99 mg, 1.12 mmol), Example 1031C (130 mg, 0.22 mmol), TEA (0.3 mL, 2.15 mmol). AcOH (0.5 mL, 8.73 mmol) and sodium cyanoborohydride (28.3 mg, 0.45 mmol) in CD₃OD (1 mL) at 0° C. afforded the crude which was purified by preparative HPLC to afford yellow gummy solid. Isomers were separated by preparative SFC (Chiralpak AD-H) to afford two isomers.

Example 1031 (Iso-1): (27.1 mg, 20% yield) as pale-yellow solid. LC-MS Method E: RT=1.39 min, [M+H]⁺=613.3; LC-MS Method F: RT=1.05 min, [M+H]⁺=613.3. ¹H NMR (400 MHz, DMSO-d₆) δ=8.65 (d, J=5.3 Hz, 1H), 8.32 (d, J=5.5 Hz, 2H), 8.03 (s, 1H), 7.39 (s, 1H), 7.22 (s, 1H), 6.93 (s, 1H), 5.11 (br s, 1H), 4.18 (br s, 1H), 3.97 (s, 3H), 2.99 (br s, 1H), 2.84 (br s, 1H), 2.77 (br s, 1H), 2.61 (s, 3H), 2.14 (br s, 2H), 1.91 (s, 1H), 1.84 (br s, 2H), 1.76 (br s, 1H), 1.69 (br s, 3H), 1.63-1.39 (m, 3H), 1.27-1.18 (m, 1H).

Example 1032 (Iso-2): (22.9 mg, 17% yield) as pale-yellow solid. LC-MS Method E: RT=1.38 min, [M+H]⁺=613.3; LC-MS Method F: RT=1.04 min, [M+H]⁺=613.3. ¹H NMR (400 MHz, DMSO-d₆) δ=8.65 (d, J=5.5 Hz, 1H), 8.32 (d, J=5.3 Hz, 2H), 8.03 (s, 1H), 7.43 (s, 1H), 7.33 (br s, 1H), 6.94 (br s, 1H), 4.82 (br s, 1H), 4.16 (br s, 1H), 3.97 (s, 3H), 3.18 (br d, J=4.8 Hz, 3H), 2.98 (br s, 3H), 2.61 (s, 3H), 2.21 (br s, 2H), 1.94-1.84 (m, 3H), 1.54-1.43 (m, 4H), 1.24 (s, 1H), 1.17-1.05 (m, 1H).

### Example 1033

### Preparation of N—((R)-1-(4-amino-7-(2-(methoxy-d3)pyrimidin-4-yl)pyrrolo[2,1-f][1,2,4]triazin-5-yl)piperidin-3-yl)-4-(((1S,4S)-4-(dimethylamino)cyclohexyl)oxy)-2-methylthiazole-5-carboxamide

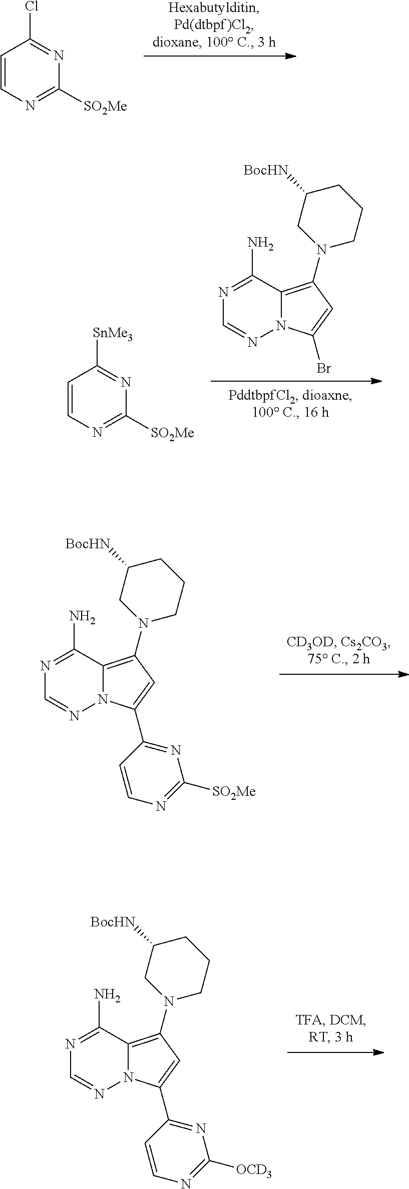

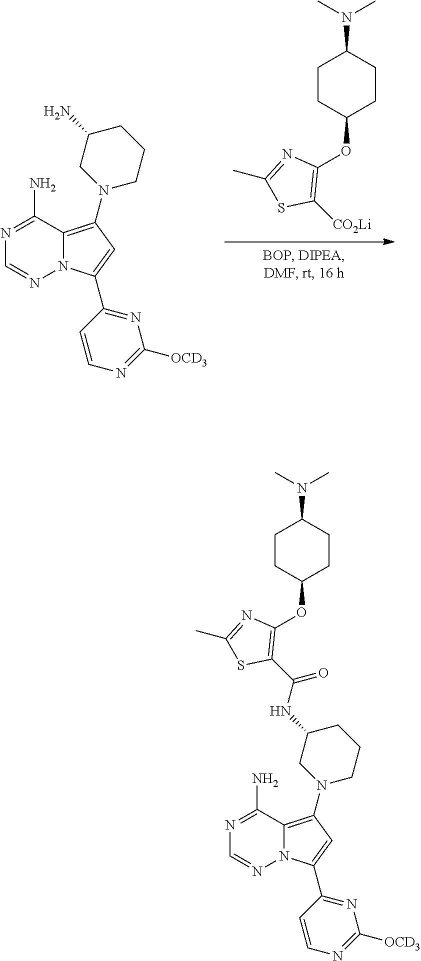

### Example 1033A. Preparation of 2-(methylsulfonyl)-4-(trimethylstannyl)pyrimidine

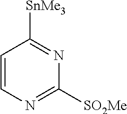

Analogous to the preparation of Example 1026C, reaction of 4-chloro-2-(methylsulfonyl)pyrimidine (1.0 g, 5.19 mmol), hexamethylditin (1.40 mL, 6.75 mmol) and Pd(dtbpf)Cl₂ in dioxane (20 mL) afforded Example 1033A (1.5 g, 90% yield) as brown gummy solid which was used as such for next step. MS: [M+H]⁺=323.0.

### Example 1033B. Preparation of tert-butyl (R)-(1-(4-amino-7-(2-(methylsulfonyl)pyrimidin-4-yl)pyrrolo[2,1-f][1,2,4]triazin-5-yl)piperidin-3-yl)carbamate

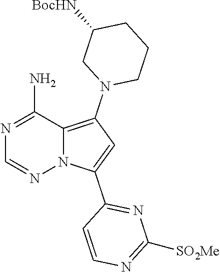 [SMILES: CC(C)(C)OC(=O)N[C@@H]1CCCN(c2cc(-c3ccnc(S(C)(=O)=O)n3)n3ncnc(N)c23)C1]

Analogous to the preparation of Example 1028E, reaction of tert-butyl (R)-(1-(4-amino-7-bromopyrrolo[2,1-f][1,2,4]triazin-5-yl)piperidin-3-yl)carbamate (2.0 g, 4.86 mmol), Example 1033A (2.03 g, 6.32 mmol) and Pd(dtbpf)Cl₂ in dioxane (16 mL) afforded Example 1033B (1.4 g, 59% yield) as brown solid. MS: [M−H]⁺=487.2.

### Example 1033C. Preparation of tert-butyl (R)-(1-(4-amino-7-(2-(methoxy-d3)pyrimidin-4-yl)pyrrolo[2,1-f][1,2,4]triazin-5-yl)piperidin-3-yl)carbamate

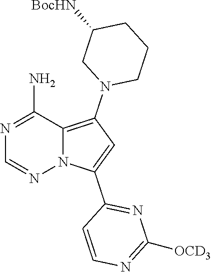

To a solution of Example 1033B (1.0 g, 1.02 mmol) in CD₃OD (5 mL) was added Cs₂CO₃ (0.66 g, 2.04 mmol) and heated at 75° C. for 2 h. The reaction was diluted with water and extracted with THF/EtOAc. The combined organic layers were washed with water, brine and dried over anhydrous Na₂SO₄. The resultant organic portion then filtered and concentrated to afford brown gummy solid, which was purified by silica gel chromatography (40 g Redisep® column, eluting with 0-100% EtOAc in hexane) to afford the Example 1033C (180 mg, 40% yield) as yellow gummy solid. MS: [M+H]⁺=445.15. ¹H NMR (400 MHz, DMSO-d₆) δ=8.64 (d, J=5.0 Hz, 1H), 8.31 (d, J=5.0 Hz, 1H), 8.19 (br d, J=2.5 Hz, 1H), 8.01 (s, 1H), 7.38 (s, 1H), 4.04 (q, J=7.3 Hz, 1H), 3.68 (br s, 2H), 3.10 (br d, J=9.0 Hz, 1H), 2.68 (br s, 2H), 1.81 (br s, 3H), 1.68 (br s, 1H), 1.39 (s, 9H).

### Example 1033D. Preparation of (R)-5-(3-aminopiperidin-1-yl)-7-(2-(methoxy-d3)pyrimidin-4-yl)pyrrolo[2,1-f][1,2,4]triazin-4-amine. TFA

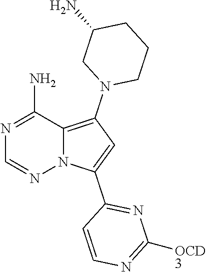

Analogous to the procedure for the preparation of Example 1002E, reaction of Example 1033C (180 mg, 0.41 mmol) and TFA (0.47 mL, 6.09 mmol) afforded Example 1033D (150 mg, 81% yield) as yellow solid. MS: [M+H]⁺=344.3; ¹H NMR (400 MHz, DMSO-d₆) δ=8.66 (d, J=5.5 Hz, 1H), 8.40-8.21 (m, 2H), 8.07-8.00 (m, 3H), 7.41 (s, 1H), 7.19 (br d, J=17.5 Hz, 1H), 3.34-3.22 (m, 1H), 3.15 (br s, 1H), 2.89 (br s, 2H), 2.74 (br s, 1H), 1.95 (br s, 1H), 1.88 (br s, 1H), 1.71 (br s, 2H).

### Example 1033

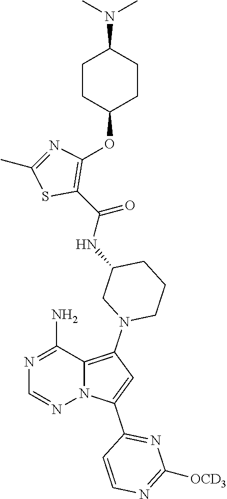

Analogous to the preparation of Example 996, reaction of Example 1033D (92 mg, 0.24 mmol), Example 1028C (70 mg, 0.24 mmol), BOP (128 mg, 0.29 mmol) and DIPEA (0.13 mL, 0.72 mmol) in DMF (3 mL) at rt for 16 h afforded the crude which was purified via preparative LC/MS with the following conditions: Column: Waters XBridge C18, 150 mm×19 mm, 5-μm particles: Mobile Phase A: 5:95 acetonitrile: water with 10-mM ammonium acetate; Mobile Phase B: 95:5 acetonitrile: water with 10-mM ammonium acetate; Gradient: a 0-minute hold at 12% B, 12-45% B over 25 minutes, then a 5-minute hold at 100% B; Flow Rate: 20 mL/min; Column Temperature: 25 C. Fraction collection was triggered by signals. Fractions containing the desired product were combined and dried via centrifugal evaporation to afford Example 1033 (32.9 mg, 21% yield) as pale-yellow solid. LC-MS Method E: RT=1.40 min, [M+H]⁺=610.3 LC-MS Method F: RT=1.05 m, [M+H]⁺=610.3; ¹H NMR (400 MHz, DMSO-d₆) δ=8.64 (d, J=5.3 Hz, 1H), 8.31 (d, J=5.3 Hz, 2H), 8.03 (s, 1H), 7.39 (s, 1H), 7.24 (br s, 1H), 6.95 (s, 1H), 5.12 (br s, 1H), 4.19 (br s, 1H), 3.23-3.13 (m, 1H), 2.99 (br s, 1H), 2.84 (br s, 1H), 2.77 (br s, 1H), 2.72-2.64 (m, 1H), 2.63-2.53 (m, 4H), 2.38-2.29 (m, 2H), 2.26 (br s, 2H), 2.08 (br s, 3H), 1.98-1.90 (m, 1H), 1.84 (br s, 2H), 1.75-1.57 (m, 5H), 1.52 (br s, 3H), 1.24 (s, 1H).

### Example 1034

### Preparation of N—((R)-1-(4-amino-7-(2-isopropoxypyrimidin-4-yl)pyrrolo[2,1-f][1,2,4]triazin-5-yl)piperidin-3-yl)-4-((1S,4S)-4-(dimethylamino)cyclohexyl)oxy)-2-methylthiazole-5-carboxamide

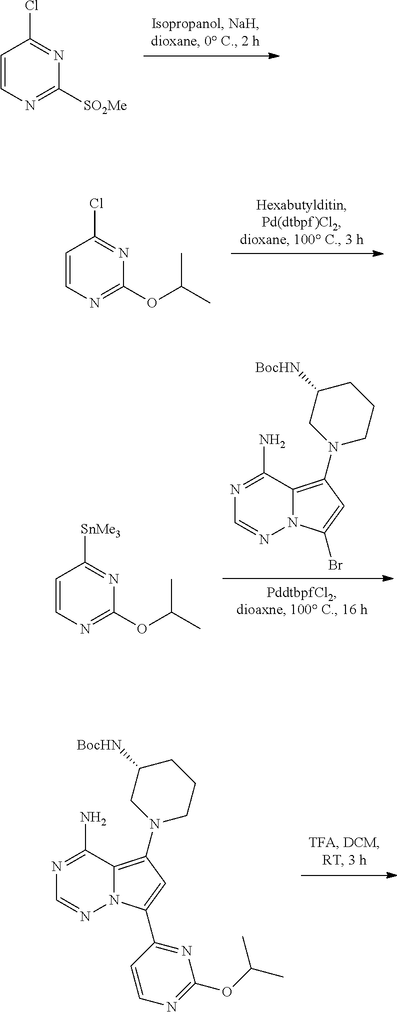

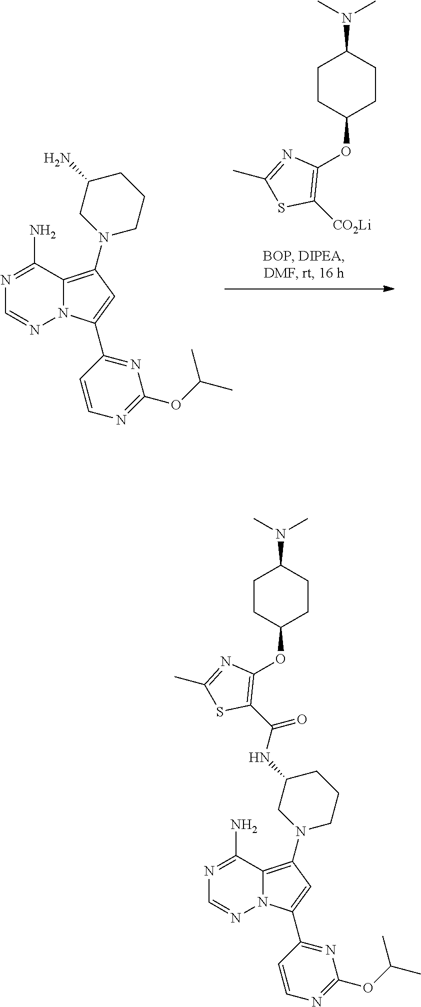

### Example 1034A. Preparation of 4-chloro-2-isopropoxypyrimidine

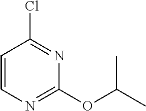

Sodium hydride (0.45 g, 18.69 mmol) was added to a solution of 2-propanol (2.40 mL, 31.1 mmol) in dioxane (30 mL) at 0° C. and stirred for 30 min. followed by the addition of 4-chloro-2-(methylsulfonyl)pyrimidine (1.4 g, 52% yield). The mixture was stirred at rt for 2 h under nitrogen. Then the reaction was quenched with ice cold water and partitioned between water (50 mL) and ethyl acetate (50 mL). The organic layer was separated, dried over sodium sulphate and concentrated under reduced pressure. The residue was purified by silica gel chromatography (120 g Redisep® column, eluting with 0-100% EA in hexane, compound eluted at 20-30%). to afford Example 1034A (1.4 g, 52% yield) as yellowish liquid. MS: [M+H]⁺=173.1. ¹H NMR (300 MHz, CHLOROFORM-d) δ=8.29 (d, J=5.2 Hz, 1H), 6.86 (d, J=5.1 Hz, 1H), 5.33-5.13 (m, 1H), 1.35-1.31 (m, 6H).

### Example 1034B. Preparation of 5-(difluoromethyl)-3-(1-methyl-1,2,3,6-tetrahydropyridin-4-yl)thiophene-2-carboxylic acid

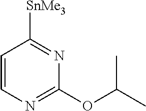

Analogous to the preparation of Example 1016C, reaction of Example 1034A (1.4 g, 8.11 mmol) and hexamethylditin (2.19 mL, 10.54 mmol) in dioxane (15 mL) afforded Example 1034B (2.0 g, 82% yield) as brown gummy solid (which was used as such for next step). MS: [M+H]⁺=303.0.

### Example 1034C. Preparation of ten-butyl (R)-(1-(4-amino-7-(2-isopropoxypyrimidin-4-yl)pyrrolo[2,1-f][1,2,4]triazin-5-yl)piperidin-3-yl)carbamate

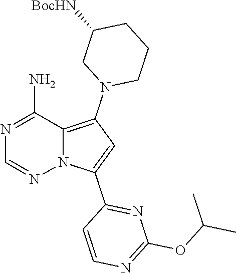 [SMILES: CC(C)Oc1nccc(-c2cc(N3CCC[C@@H](NC(=O)OC(C)(C)C)C3)c3c(N)ncnn23)n1]

Analogous to the preparation of Example 1028E, reaction of tert-butyl (R)-(1-(4-amino-7-bromopyrrolo[2,1-f][1,2,4]triazin-5-yl)piperidin-3-yl)carbamate (1.5 g, 3.65 mmol), Example 1034B (1.43 g, 4.74 mmol) and [1,1′-bis(di-tert-butylphosphino)ferrocene]dichloropalladium(II) (0.24 g, 0.36 mmol) in dioxane (20 mL) afforded residue which was purified by silica gel chromatography (40 g Redisep® column, eluting with 0-100% EtOAc in DCM) to afford Example 1034C (460 mg, 27% yield) as yellow solid. MS: [M+H]⁺=469.20; ¹H NMR (400 MHz, DMSO-d₆) δ=8.61 (d, J=5.0 Hz, 1H), 8.27 (d, J=5.0 Hz, 1H), 8.19 (br s, 1H), 8.01 (s, 1H), 7.33 (s, 1H), 7.12 (br s, 2H), 5.30 (quin, J=6.3 Hz, 1H), 4.44-4.33 (m, 2H), 3.10 (br d, J=8.5 Hz, 1H), 2.90 (br s, 1H), 2.74 (br s, 1H), 2.68 (br s, 1H), 1.81 (br s, 1H), 1.68 (br s, 1H), 1.94-1.60 (m, 1H), 1.99 (s, 1H), 1.39 (s, 9H), 1.35 (d, J=6.0 Hz, 6H).

### Example 1034D. Preparation of (R)-5-(3-aminopiperidin-1-yl)-7-(2-isopropoxypyrimidin-4-yl)pyrrolo[2,1-f][1,2,4]triazin-4-amine. TFA

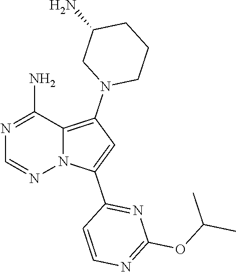 [SMILES: CC(C)Oc1nccc(-c2cc(N3CCC[C@@H](N)C3)c3c(N)ncnn23)n1]

Analogous to the preparation of Example 1002E, reaction of Example 1034C (460 mg, 0.98 mmol) and TFA (1.13 mL, 15.73 mmol) in DCM (5 mL) afforded Example 1034D (420 mg, 89% yield) as yellow solid. MS: [M+H]⁺=369.20; ¹H NMR (400 MHz, DMSO-d₆) δ=8.64 (d, J=5.0 Hz, 1H), 8.36-8.30 (m, 1H), 8.27 (d, J=5.5 Hz, 1H), 8.09-7.97 (m, 4H), 7.38 (s, 1H), 7.23-7.10 (m, 1H), 5.30 (t, J=6.0 Hz, 1H), 3.25 (br d, J=12.0 Hz, 1H), 2.93 (br s, 2H), 1.72 (br s, 2H), 1.36 (d, J=6.0 Hz, 7H), 1.27 (br d, J=13.5 Hz, 2H).

### Example 1034

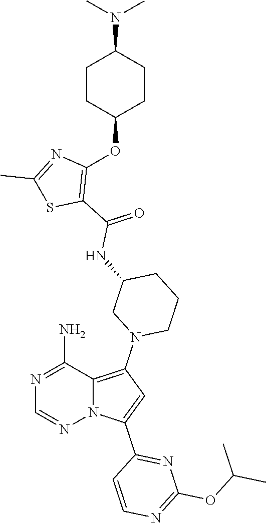 [SMILES: Cc1nc(O[C@H]2CC[C@@H](N(C)C)CC2)c(C(=O)N[C@@H]2CCCN(c3cc(-c4ccnc(OC(C)C)n4)n4ncnc(N)c34)C2)s1]

Analogous to the preparation of Example 996, reaction of Example 1034D (112 mg, 0.27 mmol), Example 1028C (80 mg, 0.27 mmol), BOP (146 mg, 0.33 mmol) and DIPEA (0.14 mL, 0.83 mmol) in DMF (3 mL) afforded the residue which was purified Va preparative LC/MS with the following conditions: Column: Xbridge C18, 0×0 mm, 0-μm particles; Mobile Phase A: 10 mM Ammonium Bicarbonate PH-9.5; Mobile Phase B: acetonitrile; Gradient: 10-40% B over 10 minutes, then a 5-minute hold at 40% B; Flow: mL/min. Fractions containing the desired product were combined and dried via centrifugal evaporation to afford Example 1034 (27.1 mg, 15% yield) as pale yellow solid. LC-MS Method E: RT=1.67 min, [M+H]⁺=635.3; LC-MS Method F: RT=1.25 min, [M+H]⁺=635.3; ¹H NMR (400 MHz, DMSO-d₆) δ=8.61 (d, J=5.3 Hz, 1H), 8.26 (d, J=5.3 Hz, 2H), 8.01 (s, 1H), 7.35 (s, 1H), 7.26 (br s, 1H), 6.97 (br s, 1H), 5.29 (quin, J=6.2 Hz, 1H), 5.13 (br s, 1H), 4.19 (br s, 1H), 3.23 (br s, 1H), 2.91 (br s, 2H), 2.60 (s, 3H), 2.29-1.90 (m, 9H), 1.84 (br s, 2H), 1.73-1.41 (m, 6H), 1.35 (d, J=6.3 Hz, 6H).

### Example 1035

### Preparation of N—((R)-1-(4-amino-7-(2-methylpyrimidin-4-yl)pyrrolo[2,1-f][1,2,4]triazin-5-yl)piperidin-3-yl)-4-(((1S,4S)-4-(dimethylamino)cyclohexyl)oxy)-2-methylthiazole-5-carboxamide

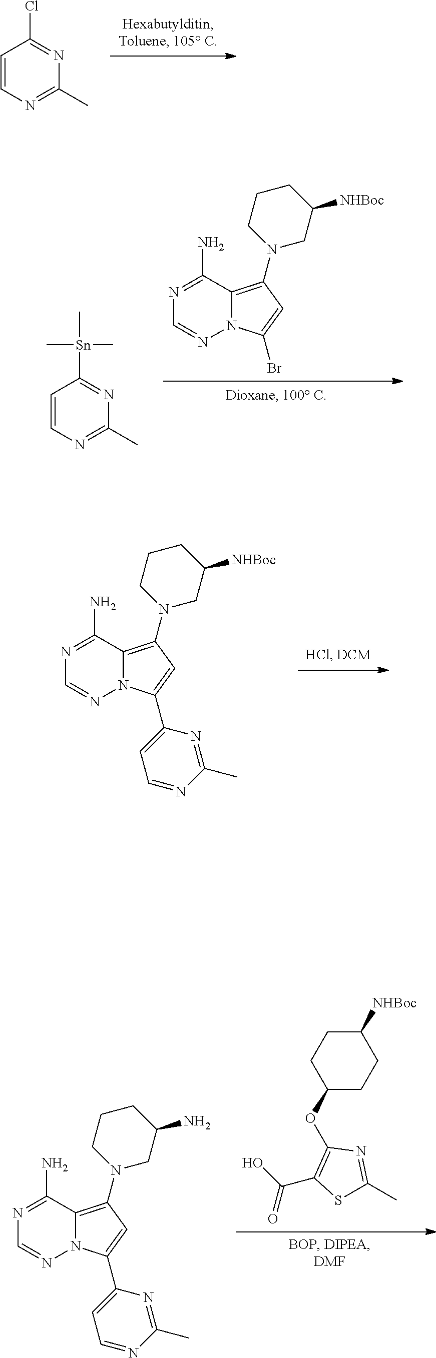

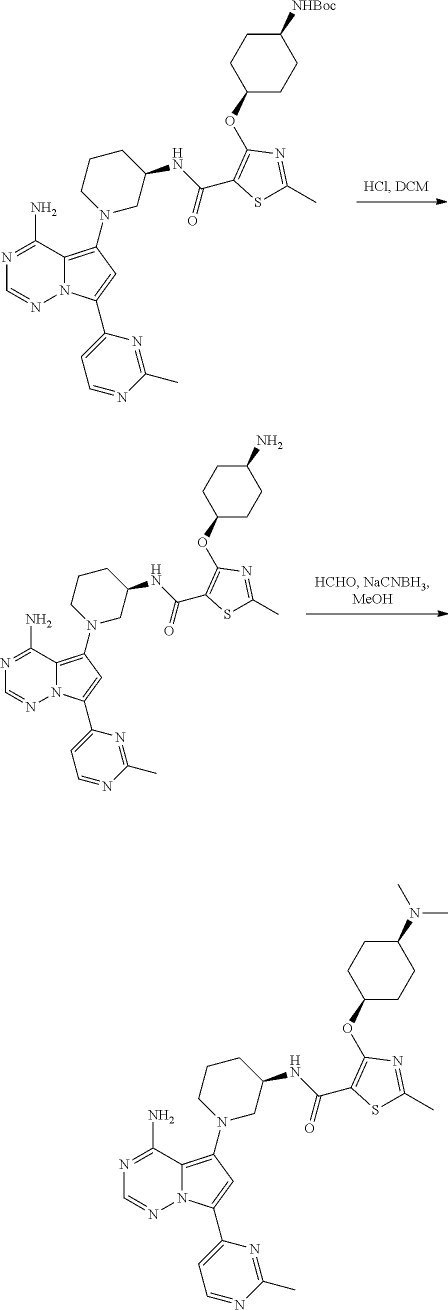

### Example 1035A: Preparation of 2-methyl-4-(trimethylstannyl)pyrimidine

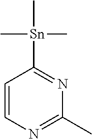

Analogous to the preparation of Example 1028C, reaction of 4-chloro-2-methylpyrimidine (500 mg, 3.89 mmol), 1,1,1,2,2,2-hexamethyldistannane (1657 mg, 5.06 mmol) and 1′-bis(di-tert-butylphosphino)ferrocene-paladium dichloride (253 mg, 0.39 mmol) in anhydrous toluene (20 mL) at 105° C. for 3 h afforded Example 1035A (999 mg, 100% yield) product as brown oil. MS: [M+H]⁺=258.8

### Example 1035B: Preparation of tert-butyl (R)-(1-(4-amino-7-(2-methylpyrimidin-4-yl)pyrrolo[2,1f][1,4]triazin-5-yl)piperidin-3-yl)carbamate

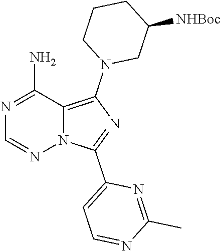 [SMILES: Cc1nccc(-c2nc(N3CCC[C@@H](NC(=O)OC(C)(C)C)C3)c3c(N)ncnn23)n1]

Analogous to the preparation of Example 1028E, reaction of Example 1035A (999 mg, 3.89 mmol), tert-butyl (R)-(1-(4-amino-7-bromopyrrolo[2,1-f][1,2,4]triazin-5-yl)piperidin-3-yl)carbamate (1439 mg, 3.50 mmol) and [1,1-bis(di-tert-butylphosphino)ferrocene]dichloropalladium(II) (253 mg, 0.39 mmol) in anhydrous dioxane (15 mL) at 100° C. for 16 h afforded the residue was purified using CombiFlash (silica gel 60-120 mesh; 4% methanol in DCM as eluent) to afford Example 1035B (310 mg, 19% yield) as a brown solid; ¹H NMR (300 MHz, DMSO-d₆) δ=8.72 (d, J=5.7 Hz, 1H), 8.46 (d, J=5.7 Hz, 1H), 8.24-8.10 (m, 1H), 8.01 (s, 1H), 7.35 (s, 1H), 6.83 (ddd, J=1.3, 3.5, 4.9 Hz, 1H), 6.74-6.66 (m, 1H), 3.76-3.59 (m, 1H), 3.23-3.03 (m, 2H), 2.98-2.69 (m, 2H), 2.64 (s, 3H), 1.96-1.55 (m, 4H), 1.39 (s, 9H). MS: [M+H]⁺=425.3

### Example 1035C: Preparation of (R)-5-(3-aminopiperidin-1-yl)-7-(2-methylpyrimidin-4-yl)pyrrolo[2,1-f][1,2,4]triazin-4-amine. HCl

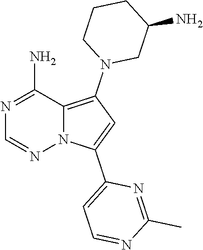 [SMILES: Cc1nccc(-c2cc(N3CCC[C@@H](N)C3)c3c(N)ncnn23)n1]

Analogous to the preparation of Example 996G, reaction of Example 1035B (300 mg, 0.71 mmol) with 4M dioxane HCl (2 mL, 8.0 mmol) in anhydrous DCM (2 mL) at rt for 1 h afforded Example 1035C (230 mg, 90% yield) as a brown solid. MS: [M+H]⁺=325.0.

### Example 1035D: Preparation of tert-butyl ((1S,4S)-4-((5-(((R)-1-(4-amino-7-(2-methylpyrimidin-4-yl)pyrrolo[2,1-f][1,2,4]triazin-5-yl)piperidin-3-yl)carbamoyl)-2-methylthiazol-4-yl)oxy)cyclohexyl)carbamate

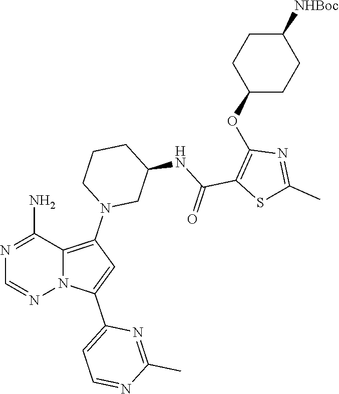 [SMILES: Cc1nccc(-c2cc(N3CCC[C@@H](NC(=O)c4sc(C)nc4O[C@H]4CC[C@@H](NC(=O)OC(C)(C)C)CC4)C3)c3c(N)ncnn23)n1]

Analogous to the preparation of Example 996, reaction of Example 1035C (230 mg, 0.71 mmol), BOP (533 mg, 1.2 mmol), Example 1002B (329 mg, 0.92 mmol) and DIPEA (0.74 mL, 4.25 mmol) in anhydrous DMF (6 mL) at rt afforded Example 1035D (420 mg, 63% yield) as a pale yellow solid. MS: [M+H]⁺=663.6.

### Example 1035E: Preparation of N—((R)-1-(4-amino-7-(2-methylpyrimidin-4-yl)pyrrolo[2,1-f][1,2,4]triazin-5-yl)piperidin-3-yl)-4-(((1S,4S)-4-aminocyclohexyl)oxy)-2-methylthiazole-5-carboxamide

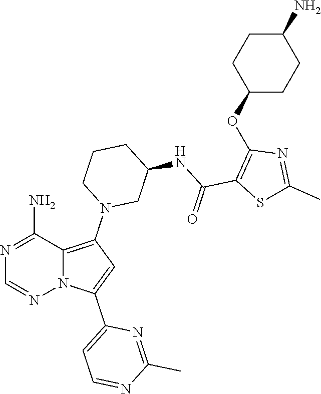 [SMILES: Cc1nccc(-c2cc(N3CCC[C@@H](NC(=O)c4sc(C)nc4O[C@H]4CC[C@@H](N)CC4)C3)c3c(N)ncnn23)n1]

Analogous to the preparation of Example 996G, reaction of Example 1035D (420 mg, 0.63 mmol) with 4M HCl in dioxane (1.58 mL, 6.34 mmol) in anhydrous DCM (3 mL) at rt afforded Example 1035E (350 mg, 59% yield) as a pale yellow solid. MS: [M+H]⁺=563.4.

### Example 1035

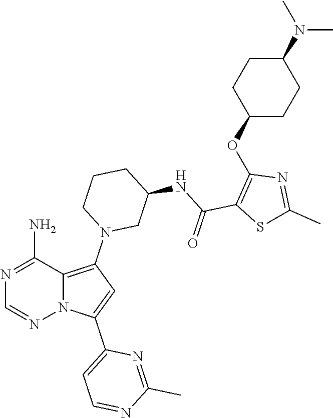 [SMILES: Cc1nccc(-c2cc(N3CCC[C@@H](NC(=O)c4sc(C)nc4O[C@H]4CC[C@@H](N(C)C)CC4)C3)c3c(N)ncnn23)n1]

Analogous to the preparation of Example 996B, reaction of Example 1035E (350 mg, 0.62 mmol), 37% formaldehyde (505 mg, 6.22 mmol) and sodium cyanoborohydride (195 mg, 3.11 mmol) in anhydrous methanol (6 mL) at rt for 2 h afforded the crude which was purified by preparative HPLC to afford Example 1035 (75 mg, 18% yield) as a yellow solid. LC-MS Method E: RT=1.60 min, [M+H]⁺=591.3; ¹H NMR (400 MHz, DMSO-d₆) δ=9.33 (br d, J=3.1 Hz, 1H), 8.74 (d, J=5.5 Hz, 1H), 8.47 (d, J=5.4 Hz, 1H), 8.36-8.15 (m, 1H), 8.02 (s, 1H), 7.38 (s, 1H), 7.26-6.83 (m, 2H), 5.10 (br s, 1H), 4.20-4.07 (m, 1H), 3.09-2.80 (m, 4H), 2.64 (s, 3H), 2.60 (s, 6H), 2.25 (br d, J=14.4 Hz, 2H), 1.98-1.38 (m, 12H).

### Example 1036

### Preparation of N—((R)-1-(4-amino-7-(2-methylpyrimidin-4-yl)pyrrolo[2,1-f][1,2,4]triazin-5-yl)piperidin-3-yl)-4-(((1S,4S)-4 (isopropylamino)cyclohexyl)oxy)-2-methylthiazole-5-carboxamide

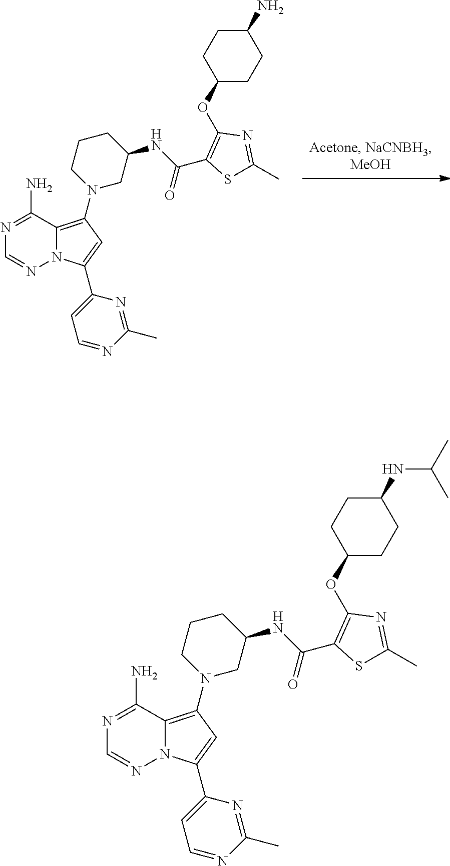 [SMILES: Cc1nccc(-c2cc(N3CCC[C@@H](NC(=O)c4sc(C)nc4O[C@H]4CC[C@@H](N)CC4)C3)c3c(N)ncnn23)n1 ; "Acetone, NaCNBH3,MeOH" ; Cc1nccc(-c2cc(N3CCC[C@@H](NC(=O)c4sc(C)nc4O[C@H]4CC[C@@H](NC(C)C)CC4)C3)c3c(N)ncnn23)n1]

Analogous to the preparation of Example 996B, reaction of Example 1035E (50 mg, 0.09 mmol), acetone (0.03 mL, 0.44 mmol) and sodium cyanoborohydride (27.9 mg, 0.44 mmol) in anhydrous methanol (3 mL) at rt for 2 h afforded the crude which was purified by preparative HPLC to afford Example 1036 (8.2 mg, 15% yield) as a yellow solid. LC-MS Method F: RT=1.02 min, [M+H]⁺=605.0; ¹H NMR (400 MHz, DMSO-d₆) δ=8.73 (d, J=5.5 Hz, 1H), 8.47 (d, J=5.3 Hz, 1H), 8.28 (br s, 1H), 8.02 (s, 1H), 7.39 (s, 1H), 7.28 (br s, 1H), 6.94 (s, 1H), 5.07 (br s, 1H), 4.17 (br s, 1H), 2.94 (br s, 3H), 2.63 (s, 3H), 2.61 (s, 3H), 2.08 (s, 2H), 2.00-1.77 (m, 3H), 1.76-1.52 (m, 101H), 1.42 (br s, 1H), 0.95 (br s, 6H).

### Example 1037

### Preparation of N—((R)-1-(4-amino-7-(2-methylpyrimidin-4-yl)pyrrolo[2,1-f][1,2,4]triazin-5-yl)piperidin-3-yl)-4-(((1S,4S)-4-(azetidin-1-yl)cyclohexyl)oxy)-2-methylthiazole-5-carboxamide

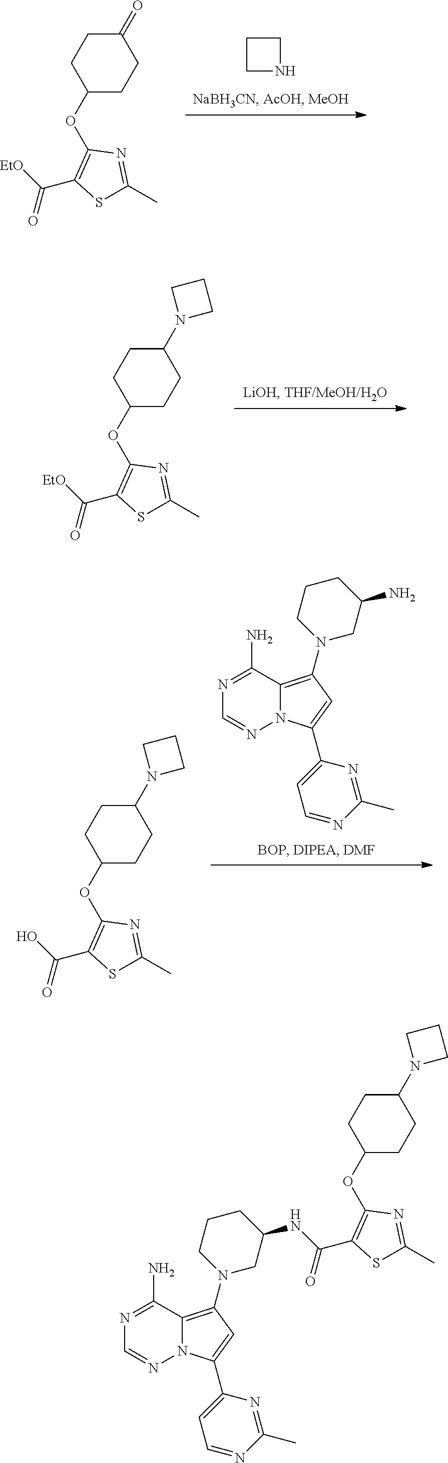

### Example 1037A and Example 1037B. Preparation of ethyl 4-((4-(azetidin-1-yl)cyclohexyl)oxy)-2-methylthiazole-5-carboxylate

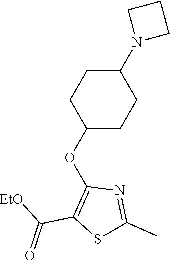 [SMILES: CCOC(=O)c1sc(C)nc1OC1CCC(N2CCC2)CC1]

Analogous to the preparation of Example 1000A, reaction of Example 941B (2.0 g, 7.06 mmol), azetidine (2.37 mL, 35.3 mmol), titanium(IV) isopropoxide (4.18 mL, 14.12 mmol) and NaBH₄ (0.40 g, 10.59 mmol) in THF (5 mL) at rt for 16 h afforded the crude which was purified by column chromatography (120 g silica gel column, 0-20% of methanol in DCM, compound eluted at 15-16%) to afford yellow gummy solid, which was purified by SFC (Preparative SFC Conditions; Column/dimensions: Lux Cellulose-1(250×30) mm, 5u; % CO₂: 85%; % Co solvent: 15% of 0.2% Methanolic ammonia in methanol; Total Flow: 125.0 g/min; Back; Pressure: 120bar; Temperature: 35° C.; UV: 240 nm) to afford Example 1037A (Iso-1, 500 mg, 22% yield) as yellow gummy solid MS. [M+H]⁺=325.20. 100% ee; ¹H NMR (400 MHz, CHLOROFORM-d) δ=5.11-5.05 (m, 1H), 4.29 (q, J=7.3 Hz, 2H), 3.18 (t, J=6.8 Hz, 4H), 2.61 (s, 3H), 2.43-2.17 (m, 1H), 2.14-1.99 (m, 5H), 1.60-1.51 (m, 5H), 1.36 (t, J=7.3 Hz, 3H) and Example 1037B (Iso-2, 120 mg, 5% yield) as yellow gummy solid. MS. [M+H]⁺=325.25. 99% ee; ¹H NMR (400 MHz, CHLOROFORM-d) δ=4.96-4.86 (m, 1H), 4.28 (q, J=7.0 Hz, 2H), 3.24 (br t, J=6.8 Hz, 4H), 2.63 (s, 3H), 2.21-2.04 (m, 5H), 1.92-1.82 (m, 2H), 1.65-1.53 (m, 2H), 1.34 (t, J=7.0 Hz, 3H), 1.28-1.13 (m, 2H).

### Example 1037C. Preparation of lithium 4-((4-(azetidin-1-yl)cyclohexyl)oxy)-2-methylthiazole-5-carboxylic acid

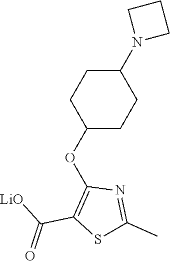

Analogous to the preparation of Example 996C, reaction of Example 1037A (500 mg, 1.54 mmol) with LiOH·H₂O (129 mg, 3.08 mmol) in ethanol (2.5 mL)/THF (2.5 mL)/water (1.5 mL) at rt for 16 h afforded the crude which was azeotroped with toluene to afford Example 1037C (420 mg, 90% yield) as white solid. MS. [M+H]⁺=297.10; ¹H NMR (400 MHz, DMSO-d₆) δ=4.78 (br t, J=7.3 Hz, 1H), 3.03 (t, J=6.8 Hz, 4H), 2.49-2.41 (m, 3H), 2.04 (br s, 1H), 1.94-1.73 (m, 4H), 1.51 (br d, J=7.0 Hz, 4H), 1.33 (br d, J=11.0 Hz, 2H).

### Example 1037

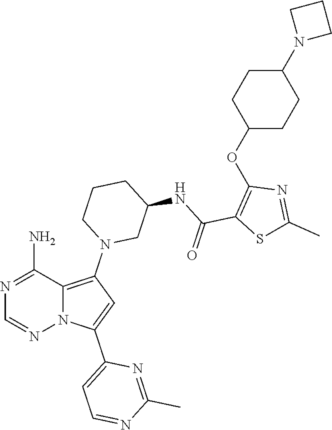 [SMILES: Cc1nccc(-c2cc(N3CCC[C@@H](NC(=O)c4sc(C)nc4OC4CCC(N5CCC5)CC4)C3)c3c(N)ncnn23)n1]

Analogous to the preparation of Example 996, reaction of Example 1035C (100 mg, 0.31 mmol), Example 1037C (91 mg, 0.31 mmol), DIPEA (0.16 mL, 0.92 mmol) and BOP (232 mg, 0.52 mmol) in anhydrous DMF (2 mL) at rt for 2 h afforded the crude which was purified by preparative HPLC to afford Example 1037 (48 mg, 26% yield) as a yellow solid. LC-MS Method E: RT=1.50 min, [M+H]⁺=603.25; ¹H NMR (400 MHz, DMSO-d₆) δ=8.72 (d, J=5.0 Hz, 1H), 8.47 (d, J=5.5 Hz, 1H), 8.02 (s, 1H), 7.44 (s, 1H), 7.40-7.24 (m, 1H), 7.07-6.87 (m, 1H), 5.08 (br s, 1H), 4.22 (br s, 1H), 3.07-2.95 (m, 5H), 2.61 (br s, 3H), 2.60 (br s, 3H), 2.05 (br d, J=8.0 Hz, 1H), 1.94-1.77 (m, 10H), 1.72-1.57 (m, 3H), 1.49 (br dd, J=6.5, 8.0 Hz, 2H), 1.37-1.21 (m, 2H).

### Example 1038

### Preparation of (R)—N-(1-(4-amino-7-(2-methoxypyridin-4-yl)pyrrolo[2,1-f][1,2,4]triazin-5-yl)piperidin-3-yl)-4-((4-(azetidin-1-yl)cyclohexyloxy)-2-methylthiazole-5-carboxamide

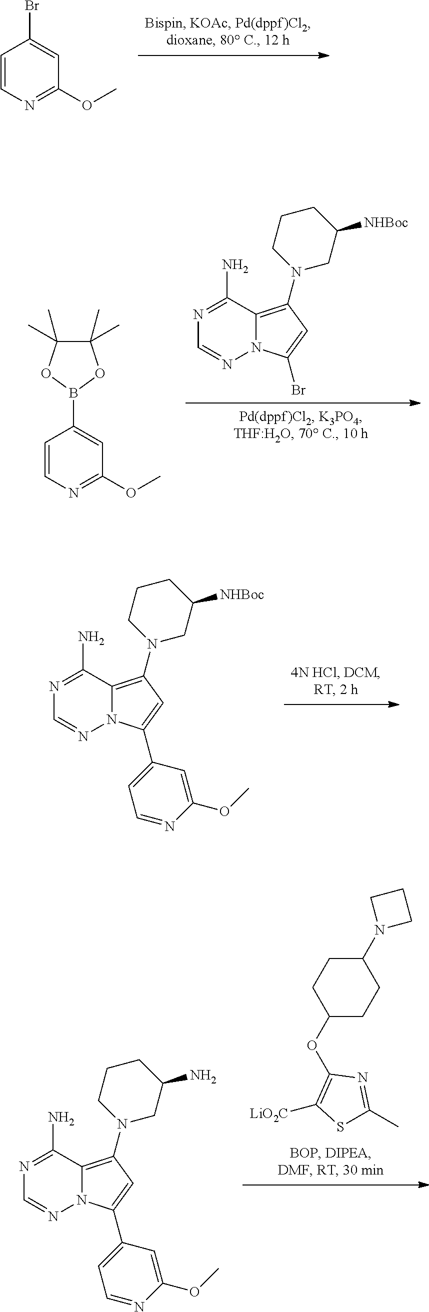

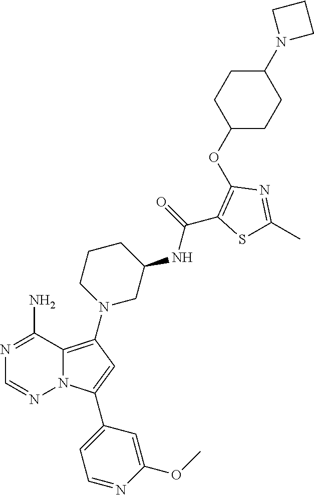 [SMILES: COc1cc(-c2cc(N3CCC[C@@H](NC(=O)c4sc(C)nc4OC4CCC(N5CCC5)CC4)C3)c3c(N)ncnn23)ccn1]

### Example 1038A: Preparation of 2-Methoxy-4-(4,4,5,5-tetramethyl-1,3,2-dioxaborolan-2-yl)pyridine

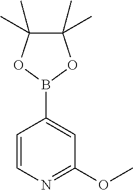

Analogous to the preparation of Example 9% E, reaction of 4-bromo-2-methoxypyridine (1.5 g, 7.98 mmol), bispin (2.431 g, 9.57 mmol), potassium acetate (2.35 g, 23.93 mmol) and Pd(dppf)Cl₂·DCM (0.32 g, 0.39 mmol) for 12 h at 80° C. in 1,4-dioxane (20 mL) afforded Example 1038A (1.75 g, 93% yield), MS: [M+H]⁺=236.2.

### Example 1038B. Preparation of tert-Butyl (R)-(1-(4-amino-7-(2-methoxypyridin-4-yl)pyrrolo[2,1-f][1,2,4]triazin-5-yl)piperidin-3-yl)carbamate

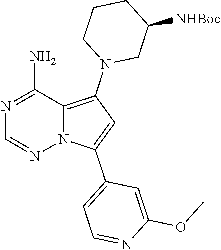 [SMILES: COc1cc(-c2cc(N3CCC[C@@H](NC(=O)OC(C)(C)C)C3)c3c(N)ncnn23)ccn1]

Analogous to the preparation of Example 9% F, reaction of tert-butyl (R)-(1-(4-amino-7-bromopyrrolo[2,1-f][1,2,4]triazin-5-yl)piperidin-3-yl)carbamate (1.5 g, 3.65 mmol), Example 1038A (1.71 g, 7.29 mmol), potassium phosphate tribasic (5.47 mL, 10.94 mmol) and Pd(dppf)Cl₂·DCM (0.29 g, 0.36 mmol) for 10 h at 70° C. in THF (20 mL)/water (2 mL) afforded the crude which was purified by using CombiFlash (RedisepRf silica gel 40 g column: 0-40% ethyl acetate in pet-ether as an eluent) to Example 1038B (1.2 g, 75% yield); MS: [M+H]⁺=440.2.

### Example 1038C. Preparation of (R)-5-(3-aminopiperidin-1-yl)-7-(2-methoxypyridin-4-yl)pyrrolo[2,1-f][1,2,4]triazin-4-amine. HCl

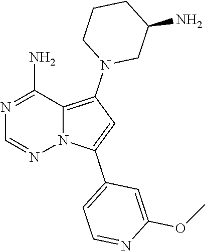 [SMILES: COc1cc(-c2cc(N3CCC[C@@H](N)C3)c3c(N)ncnn23)ccn1]

Analogous to the preparation of Example 996G, reaction of Example 1038B (1.5 g, 3.41 mmol) with 4M HCl in dioxane (4.27 ml, 17.06 mmol) in DCM (15 mL) afforded the crude which was triturated with pet-ether to afford Example 1038C (1.2 g, 94% yield) as yellow solid and taken as such without further purification; MS: [M+H]⁺=340.3.

### Example 1038

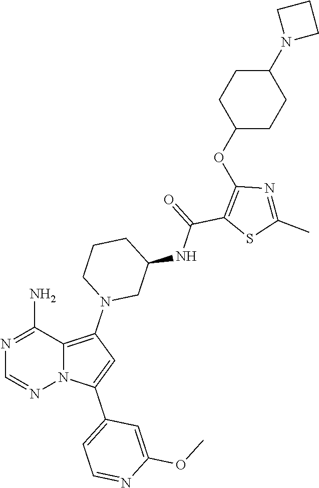 [SMILES: COc1cc(-c2cc(N3CCC[C@@H](NC(=O)c4sc(C)nc4OC4CCC(N5CCC5)CC4)C3)c3c(N)ncnn23)ccn1]

Analogous to the preparation of Example 996, reaction of Example 1037C (0.04 g, 0.15 mmol), BOP (0.07 g, 0.16 mmol), Example 1038C (0.05 g, 0.15 mmol) and DIPEA (0.10 mL, 0.61 mmol) in DMF (2 mL) at rt for 30 min. afforded the crude which was purified via preparative LC/MS using Method N to afford Example 1038 (17.3 mg, 18% yield). LC-MS Method E: RT=1.64 min, [M+H]⁺=618.3; LC-MS Method F: RT=1.11 min, MS: [M+H]⁺=618.3; ¹H NMR (400 MHz, DMSO-d₆) δ=8.18 (d, J=5.5 Hz, 2H), 7.94 (s, 1H), 7.73-7.59 (m, 2H), 7.28 (s, 2H), 6.86 (br s, 1H), 5.04 (br s, 1H), 4.19 (br s, 1H), 3.88 (s, 3H), 3.25 (br s, 2H), 2.96 (br s, 3H), 2.60 (s, 3H), 2.19-1.74 (m, 3H), 1.71-1.39 (m, 8H), 1.38-1.25 (m, 7H).

### Example 1039

### Preparation of (R)—N-(1-(4-amino-7-(4-carbamoyl-3-fluorophenyl)pyrrolo[2,1-f][1,2,4]triazin-5-yl)piperidin-3-yl)-4-((4-(3-hydroxyazetidin-1-yl)cyclohexyl)oxy)-2-methylthiazole-5-carboxamide

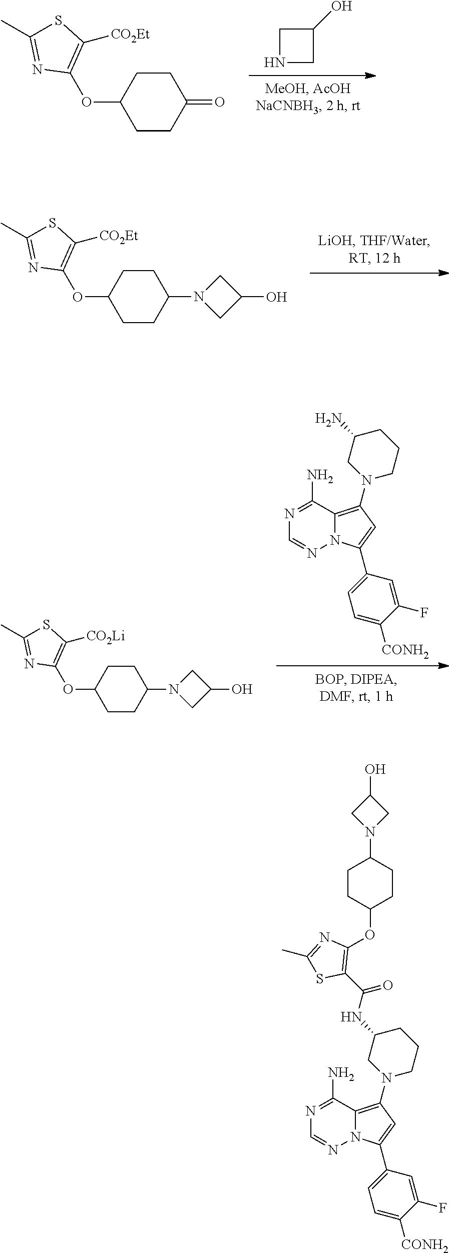

### Example 1039A. Preparation of tert-butyl 4-(((5-(difluoromethyl)-2-(methoxycarbonyl)thiophen-3-yl)oxy)methyl)piperidine-1-carboxylate

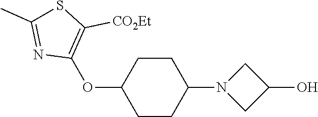 [SMILES: CCOC(=O)c1sc(C)nc1OC1CCC(N2CC(O)C2)CC1]

Analogous to the preparation of Example 996B, reaction of azetidin-3-ol (1.03 g, 14.12 mmol), Example 941B (2 g, 7.06 mmol), TEA (1.96 mL, 14.12 mmol), acetic acid (2.02 mL, 35.3 mmol) and sodium cyanoborohydride (0.88 g, 14.12 mmol) in MeOH (20 mL) afforded yellow thick residue, which was purified by SFC (Preparative SFC Conditions: Column/dimensions: Chiralpak IC (250×30) mm, 5u; % CO2: 50%, % Co solvent: 50% of 0.2% DEA in IPA; Total Flow: 150.0 g/min; Back Pressure: 100 bar; Temperature: 40° C.; UV: 240 nm) to afford two isomers.

Example 1039A (Iso-I) (526 mg, 22% yield) MS. [M+H]⁺=341.15. 100% ee; ¹H NMR (400 MHz, CHLOROFORM-d) δ=5.11 (br s, 1H), 4.47 (quin, J=5.8 Hz, 1H), 4.29 (q, J=7.0 Hz, 2H), 3.70 (dd, J=6.3, 8.3 Hz, 2H), 3.07 (br s, 1H), 3.03-2.93 (m, 2H), 2.62 (s, 3H), 2.21 (br s, 1H), 2.15-2.03 (m, 4H), 1.66-1.61 (m, 4H), 1.35 (t, J=7.0 Hz, 3H).

Example 1039B (Iso-II) (332 mg, 14% yield). MS. [M+H]⁺=341.15. 99% ee; ¹H NMR (400 MHz, CHLOROFORM-d) δ=4.96-4.88 (m, 1H), 4.44 (quin, J=5.8 Hz, 1H), 4.28 (q, J=7.2 Hz, 2H), 3.70-3.63 (m, 2H), 2.94 (dd, J=6.0, 8.5 Hz, 2H), 2.63 (s, 3H), 2.21-2.07 (m, 4H), 1.88 (br dd, J=3.3, 13.3 Hz, 3H), 1.34 (t, J=7.3 Hz, 3H).

### Example 1039C. Preparation of 3-((1-(tert-butoxycarbonyl)piperidin-4-yl)methoxy)-5-(difluoromethyl)thiophene-2-carboxylic acid

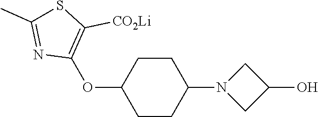

Analogous to the preparation of Example 996C, reaction of Example 1039A (400 mg, 1.17 mmol) and LiOH·H₂O (169 mg, 7.05 mmol) afforded Example 1039C (360 mg, 94% yield) as an off-white solid. MS: [M+H]⁺=313.10.

### Example 1039

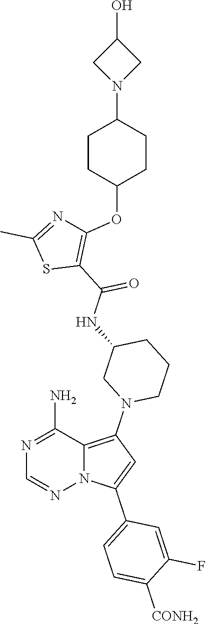 [SMILES: Cc1nc(OC2CCC(N3CC(O)C3)CC2)c(C(=O)N[C@@H]2CCCN(c3cc(-c4ccc(C(N)=O)c(F)c4)n4ncnc(N)c34)C2)s1]

Analogous to the preparation of Example 1, reaction of Example 139D (273 mg, 0.56 mmol), Example 1039C (180 mg, 0.56 mmol), BOP (297 mg, 0.67 mmol) and DIPEA (0.2 mL, 1.1 mmol) in DMF (2 mL) afforded Example 1039 (96 mg, 25% yield) as pale-yellow solid. MS: [M+H]⁺=664.15; HPLC Method G: RT=5.64 min, Method H: RT=6.91 min, ¹H NMR (400 MHz, DMSO-d₆) δ ppm 8.33-8.07 (m, 1H), 7.99 (dd, J=1.6, 8.3 Hz, 1H), 7.92 (s, 1H), 7.74 (t, J=8.1 Hz, 1H), 7.66 (s, 1H), 7.63 (s, 1H), 7.34 (br s, 1H), 7.22 (s, 1H), 6.80 (br s, 1H), 5.19 (br s, 1H), 5.06 (br s, 1H), 4.21 (br s, 1H), 4.08 (br t, J=6.1 Hz, 1H), 2.95 (br s, 2H), 2.63-2.53 (m, 6H), 2.05 (br s, 1H), 1.94-1.81 (m, 8H), 1.66 (br s, 2H), 1.49 (br s, 2H), 1.35 (br s, 2H), −0.14 (s, 1H). ¹⁹F NMR (400 MHz, DMSO-d₆) δ ppm −112.963.

### Example 1040

### Preparation of N—((R)-1-(4-amino-7-(5-oxo-2,5-dihydrofuran-3-yl)pyrrolo[2,1-f][1,2,4]triazin-5-yl)piperidin-3-yl)-4-((4-(((R)-1-methoxypropan-2-yl)amino)cyclohexyl)oxy)-2-methylthiazole-5-carboxamide

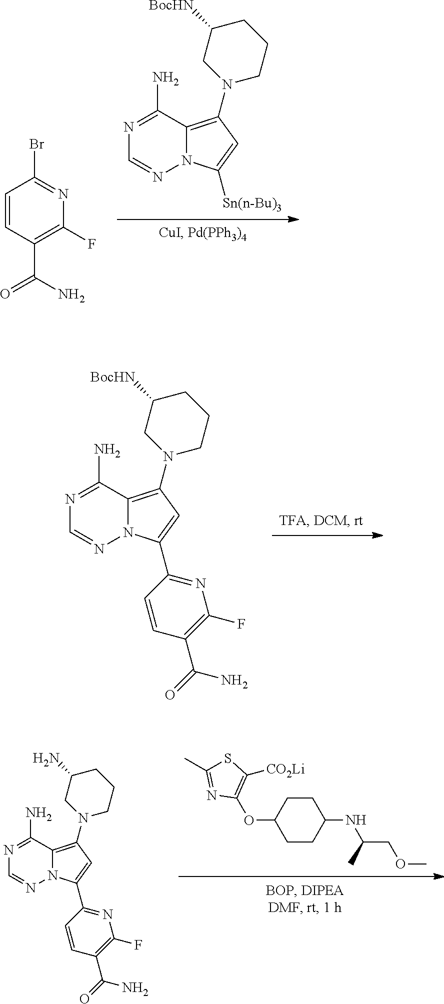

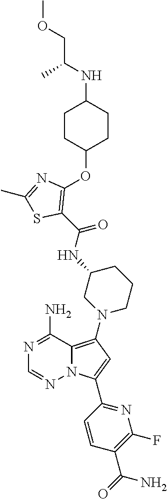 [SMILES: COC[C@@H](C)NC1CCC(Oc2nc(C)sc2C(=O)N[C@@H]2CCCN(c3cc(-c4ccc(C(N)=O)c(F)n4)n4ncnc(N)c34)C2)CC1]

### Example 1040A, Preparation of tert-butyl (R)-(1-(4-amino-7-(5-carbamoyl-6-fluoropyridin-2-yl)pyrrolo[2,1-f][1,2,4]triazin-5-yl)piperidin-3-yl)carbamate

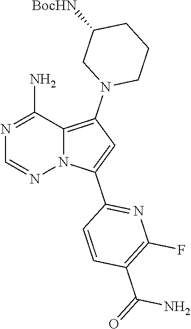 [SMILES: CC(C)(C)OC(=O)N[C@@H]1CCCN(c2cc(-c3ccc(C(N)=O)c(F)n3)n3ncnc(N)c23)C1]

Analogous to the preparation of Example 1026D, reaction of Example 956A (463 mg, 2.15 mmol), Example 1026C (1.1 g, 1.77 mmol), copper(I) iodide (33 mg, 0.17 mmol) and Pd(Ph₃P)₄ (196 mg, 0.17) in dioxane (8 mL) afforded Example 45B (700 mg, 69% yield) as yellow solid. MS: [M+H]⁺=471.25; ¹H NMR (400 MHz, DMSO-d₆) δ=8.66 (br d, J=8.0 Hz, 1H), 8.30 (dd, J=8.3, 9.8 Hz, 1H), 8.01 (br s, 1H), 7.54 (br s, 2H), 7.22 (s, 1H), 3.68 (br s, 2H), 3.48-3.39 (m, 2H), 3.09 (br s, 2H), 2.92 (br s, 1H), 2.74 (br s, 1H), 2.68 (br s, 1H), 1.84 (br d, J=18.5 Hz, 2H), 1.68 (br s, 1H), 1.39 (s, 9H).

### Example 1040B. Preparation of tert-butyl (R)-(1-(((2-((1-(7-(1-acetyl-2,5-dihydro-1H-pyrrol-3-yl)-4-aminopyrrolo[2,1-f][1,2,4]triazin-5-yl)piperidin-3-yl)carbamoyl)-5-chlorothiophen-3-yl)oxy)methyl)cyclopropyl)carbamate. TFA

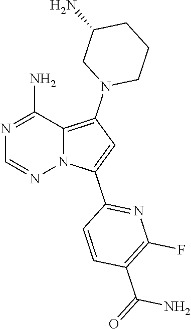 [SMILES: NC(=O)c1ccc(-c2cc(N3CCC[C@@H](N)C3)c3c(N)ncnn23)nc1F]

Analogous to the preparation of Example 1002E, reaction of Example 1040A (700 mg, 1.48 mmol) and TFA (1.71 mL, 22.32 mmol) afforded Example 1040B (640 mg, 89% yield) as yellow solid. MS: [M+H]⁺=371.30; ¹H NMR (400 MHz, DMSO-d₆) δ=8.66 (br d, J=6.5 Hz, 1H), 7.83 (br s, 1H), 7.80-7.71 (m, 1H), 7.54 (br s, 1H), 7.43 (br s, 1H), 7.28 (s, 1H), 7.20 (br s, 1H), 4.25-4.09 (m, 4H), 3.26 (br d, J=10.0 Hz, 1H), 3.01-2.74 (m, 2H), 1.96 (br s, 1H), 1.87 (br s, 1H), 1.70 (br s, 2H).

### Example 1040

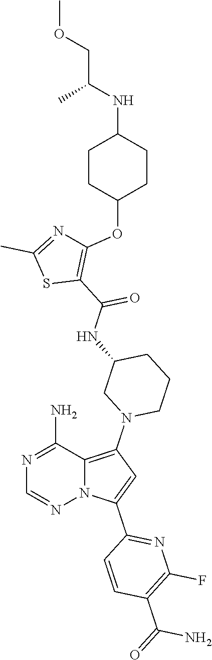 [SMILES: COC[C@@H](C)NC1CCC(Oc2nc(C)sc2C(=O)N[C@@H]2CCCN(c3cc(-c4ccc(C(N)=O)c(F)n4)n4ncnc(N)c34)C2)CC1]

Analogous to the preparation of Example 996, reaction of Example 1040B (183 mg, 0.44 mmol), Example 996C (150 mg, 0.44 mmol), BOP (238 mg, 0.53 mmol) and DIPEA (0.15 mL, 0.9 mmol) in DMF (2 mL) afforded Example 1040 (88 mg, 29% yield) as yellow solid. MS: [M+H]⁺=681.30; HPLC Method G: RT=6.52 min, Method H: RT=7.84 min, ¹H NMR (400 MHz, DMSO-d₆) δ=8.67 (dd, J=1.8, 7.9 Hz, 1H), 8.39-8.13 (m, 2H), 8.01 (s, 1H), 7.81 (br s, 1H), 7.75 (br s, 1H), 7.26 (s, 2H), 6.93 (br s, 1H), 5.11 (br s, 1H), 4.17 (br s, 1H), 3.25-3.10 (m, 5H), 3.01 (br s, 2H), 2.86 (br s, 1H), 2.76 (br s, 1H), 2.67 (br s, 1H), 2.63-2.52 (m, 5H), 1.97 (br s, 2H), 1.84 (br s, 3H), 1.75-1.50 (m, 5H), 1.47-1.17 (m, 2H), 1.09-0.80 (m, 3H), ¹⁹F NMR (400 MHz, DMSO-d₆) δ ppm −65.531.

### Example 1041

### Preparation of (R)—N-(1-(4-amino-7-(5-carbamoyl-6-fluoropyridin-2-yl)pyrrolo[2,1-f][1,2,4]triazin-5-yl)piperidin-3-yl)-4-((4-(azetidin-1-yl)cyclohexyl)oxy)-2-methylthiazole-5-carboxamide

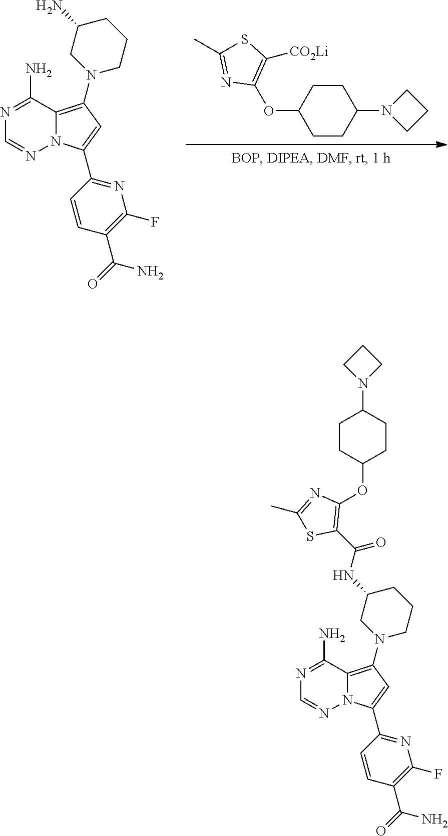

Analogous to the preparation of Example 996, reaction Example 1040B (67.3 mg, 0.16 mmol), lithium 4-((4-(azetidin-1-yl)cyclohexyl)oxy)-2-methylthiazole-5-carboxylate (50 mg, 0.16 mmol), BOP (88 mg, 0.19 mmol) and DIPEA (0.08 mL, 0.49 mmol) in DMF (3 mL) at rt 1 h afforded the crude which was purified by the reverse phase column to afford Example 1041 (16.9 mg, 15% yield) as pale yellow solid. LC-MS Method E: RT=1.26 min, [M+H]⁺=649.3; LC-MS Method F: RT=0.94 min, [M+H]⁺=649.3; ¹H NMR (400 MHz, DMSO-d₆) δ=8.66 (br d, J=7.6 Hz, 1H), 8.30 (s, 1H), 8.02 (s, 1H), 7.80 (br s, 1H), 7.74 (br s, 1H), 7.30 (s, 2H), 5.07 (br s, 1H), 4.22 (br s, 1H), 3.08-2.90 (m, 6H), 2.61-2.57 (m, 3H), 2.03 (br s, 2H), 1.94-1.75 (m, 7H), 1.63 (br d, J=12.5 Hz, 3H), 1.45 (br s, 2H), 1.29 (br s, 2H), 1.24 (br s, 1H); ¹⁹F NMR (400 MHz, DMSO-d₆) δ ppm −65.678.

### Example 1042

### Preparation of N—((R)-1-(4-amino-7-(5-oxo-2,5-dihydrofuran-3-yl)pyrrolo[2,1-f][1,2,4]triazin-5-yl)piperidin-3-yl)-2-methyl-4-((1-methyloctahydro-1H-indol-5-yl)oxy)thiazole-5-carboxamide

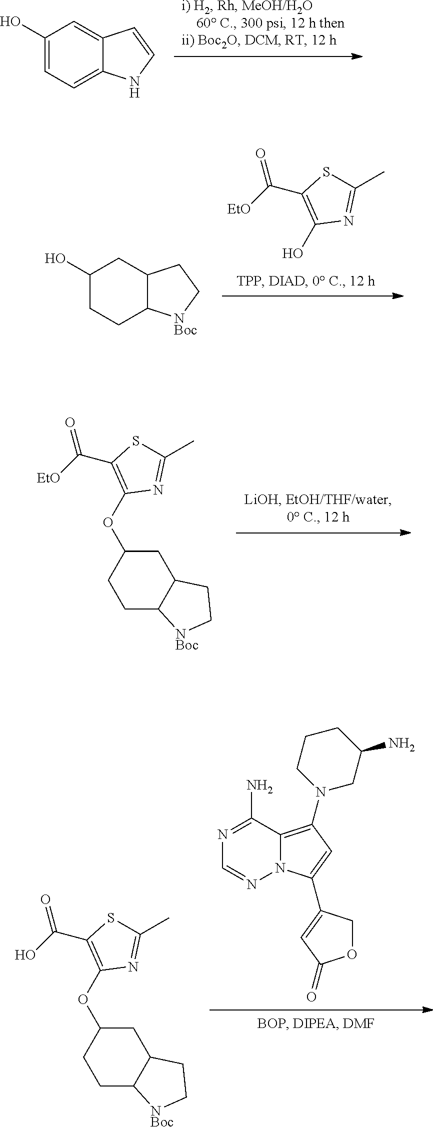

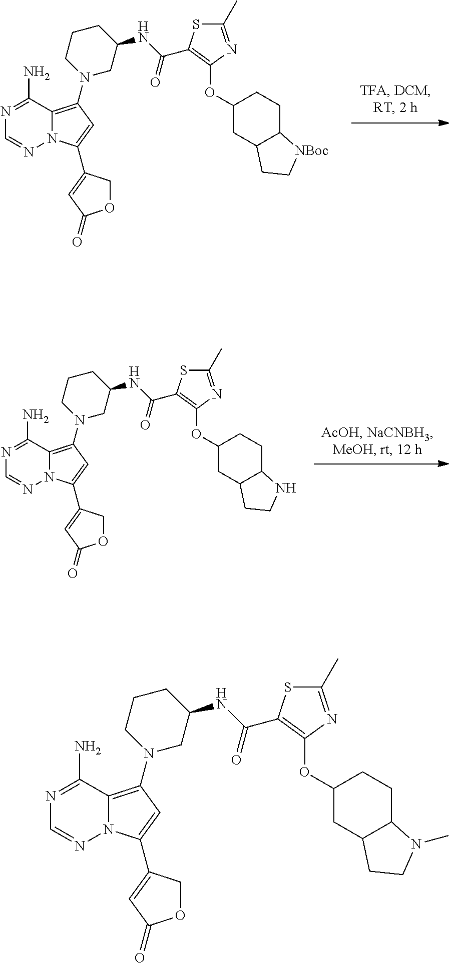 [SMILES: Cc1nc(OC2CCC3C(CCN3C(=O)OC(C)(C)C)C2)c(C(=O)N[C@@H]2CCCN(c3cc(C4=CC(=O)OC4)n4ncnc(N)c34)C2)s1 ; "TFA, DCM,RT, 2 h" ; Cc1nc(OC2CCC3NCCC3C2)c(C(=O)N[C@@H]2CCCN(c3cc(C4=CC(=O)OC4)n4ncnc(N)c34)C2)s1 ; "AcOH, NaCNBH3,MeOH, rt, 12 h" ; Cc1nc(OC2CCC3C(CCN3C)C2)c(C(=O)N[C@@H]2CCCN(c3cc(C4=CC(=O)OC4)n4ncnc(N)c34)C2)s1]

### Example 1042A: Preparation of tert-butyl (5R)-5-hydroxyoctahydro-1H-indole-1-carboxylate

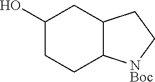 [SMILES: CC(C)(C)OC(=O)N1CCC2CC(O)CCC21]

To a stirring suspension of 5-hydroxyindole (577 mg, 4.33 mmol) in H₂O (4 mL)/MeOH (3 mL) was added 5 wt. % Rh on alumina (460 mg, 2.17 mmol) and mixture was stirred under H₂ at 300 psi for 12 h at 60° C. The reaction mixture was cooled to rt followed by release of H₂ pressure. MeOH (10 mL) was added to the reaction mixture which was subsequently filtered. The filter cake was washed with MeOH (5 mL) and the solvent from the combined filtrates was evaporated to afford a yellow oil that was taken forward without purification. To a solution of this crude in H₂O (5 mL)/THF (5 mL) was added di-tert-butyl dicarbonate (1 g, 4.33 mmol). The resulting biphasic system was stirred for 12 h and then extracted with CH₂Cl₂ (3×10 mL) and washed with saturated brine (1×10 mL). The organic portion was dried over anhydrous MgSO4 and concentrated in vacuo to get the crude. This crude product was purified by Silica gel chromatography (EtOAc in hexanes) to afford Example 1042A (500 mg, 49% yield) as a colourless oil; ¹H NMR (500 MHz, CDCl₃,) δ=3.86 (m, 1H); 3.65 (q, J=6.0 Hz, 1H); 3.42 (m, 2H); 2.22 (m, 1H); 2.18 (m, 1H); 1.94 (m, 1H); 1.85 (m, 2H); 1.72 (ddd, J=17.7, 8.4, 4.3 Hz, 1H); 1.44-1.59 (m, 3H); 1.48 (s, 9H).

### Example 1042B and Example 1042C. Preparation of ethyl 4-((1-(tert-butoxycarbonyl) octahydro-1H-indol-5-yl) oxy)-2-methylthiazole-5-carboxylate

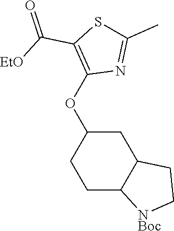 [SMILES: CCOC(=O)c1sc(C)nc1OC1CCC2C(CCN2C(=O)OC(C)(C)C)C1]

Analogous to the preparation of Example 996A, reaction of ethyl 4-hydroxy-2-methylthiazole-5-carboxylate (10 g, 53.4 mmol), Example 1042A (11.50 g, 53.4 mmol), triphenylphosphine (21.02 g, 80 mmol) and DIAD (23.89 mL, 123 mmol) in THF (100 mL) at rt for 12 h afforded the crude which was purified by flash chromatography (120 g silica gel column; 0-20% EtOAc in Pet Ether) to get the diastereomeric mixture. The two diastereomers were separated by SFC (Chiralpak IG) to afforded two isomers.

Example 1042B (Iso-1) (1.48 g, 7%) colourless oil. LC-MS Method E: RT=3.48 min, LC-MS Method E: RT=3.47 min; [M+H]⁺=411.10; Chiral SFC purity-100% at RT=2.55 min ¹H NMR (400 MHz, DMSO-d₆) δ ppm 4.98-5.08 (m, 1H) 4.74-4.82 (m, 1H) 4.15-4.23 (m, 2H) 4.02-4.14 (m, 2H) 3.18 (d, J=5.50 Hz, 5H) 2.62 (s, 3H) 1.96-2.06 (m, 2H) 1.75-1.86 (m, 4H) 1.40 (s, 9H) 1.21-1.28 (m, 3H)

Example 1042C (Iso-2) (1.2 g, 2%) colourless oil. LC-MS Method E: RT=3.47 min; [M+H]⁺=411.10. Chiral SFC purity-99% at RT=3.12 min; ¹H NMR (DMSO-d₆, 400 MHz) δ 5.0-5.1 (m, 1H), 4.1-4.2 (m, 2H), 3.6-3.7 (m, 1H), 3.2-3.3 (m, 2H), 2.6-2.7 (m, 3H), 2.3-2.5 (m, 1H), 1.9-2.1 (m, 3H), 1.7-1.9 (m, 5H), 1.40 (s, 9H), 1.24 (t, 3H, J=7.3 Hz)

### Example 1042D. Preparation of lithium 4-((1-(tert-butoxycarbonyl) octahydro-1H-indol-5-yl) oxy)-2-methylthiazole-5-carboxylate

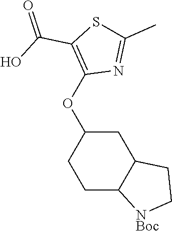 [SMILES: Cc1nc(OC2CCC3C(CCN3C(=O)OC(C)(C)C)C2)c(C(=O)O)s1]

Analogous to the preparation of Example 996C, reaction of Example 1042B (1.48 g, 3.61 mmol) and LiOH (0.26 g, 10.82 mmol) in THF (5 mL)/ethanol (5 mL)/water (5 mL) at rt for 12 h afforded Example 1042D (1.4 g, 100% yield) as a white solid. LC-MS Method J: RT=0.90 min; [M−H]⁺=381.3.

Synthesis of Example 1042E (Iso-2): Analogous to the preparation of Example 996C, reaction of Example 1042C (1.2 g, 2.92 mmol) with LiOH (0.21 g, 8.77 mmol) in THF (5 mL)/ethanol (5 mL)/water (5 mL) at rt for 12 h afforded Example 1042E (1.12 g, 98% yield) as a white solid. LC-MS Method J: RT=0.91 min; [M−H]⁺=381.2.

### Example 1042F. Preparation of tert-butyl 5-((5-(((R)-1-(4-amino-7-(5-oxo-2,5-dihydrofuran-3-yl) pyrrolo[2,1-f][1,2,4] triazin-5-yl) piperidin-3-yl) carbamoyl)-2-methylthiazol-4-yl) oxy) octahydro-1H-indole-1-carboxylate

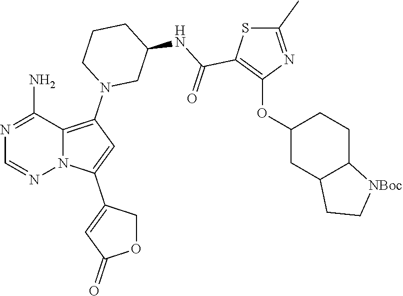 [SMILES: Cc1nc(OC2CCC3C(CCN3C(=O)OC(C)(C)C)C2)c(C(=O)N[C@@H]2CCCN(c3cc(C4=CC(=O)OC4)n4ncnc(N)c34)C2)s1]

Analogous to the preparation of Example 996, reaction of Example 996G (150 mg, 0.35 mmol), Example 1042D (136 mg, 0.35 mmol), BOP (310 mg, 0.70 mmol) and DIPEA (0.18 mL, 1.05 mmol) in DMF (2 mL) at rt for 5 h afforded crude Example 1042F (130 mg, 55% yield) [M+H]⁺=679.4

### Example 1042G. Preparation of N—((R)-1-(4-amino-7-(5-oxo-2,5-dihydrofuran-3-yl) pyrrolo[2,1-f][1,2,4] triazin-5-yl) piperidin-3-yl)-2-methyl-4-((octahydro-1H-indol-5-yl) oxy) thiazole-5-carboxamide. TFA

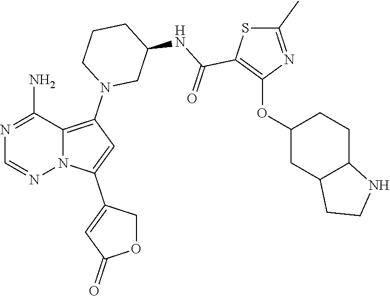 [SMILES: Cc1nc(OC2CCC3NCCC3C2)c(C(=O)N[C@@H]2CCCN(c3cc(C4=CC(=O)OC4)n4ncnc(N)c34)C2)s1]

Analogous to the preparation of Example 996G, reaction of Example 1042F (150 mg, 0.22 mmol) with TFA (0.5 mL, 6.49 mmol) in DCM (3 mL) rt for 2 h afforded Example 1042G (100 mg, 65% yield) as a light brown solid. LC-MS [M+H]⁺=579.3.

### Example 1042

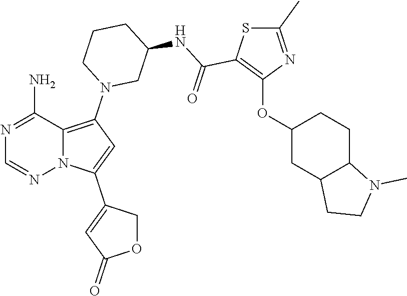 [SMILES: Cc1nc(OC2CCC3C(CCN3C)C2)c(C(=O)N[C@@H]2CCCN(c3cc(C4=CC(=O)OC4)n4ncnc(N)c34)C2)s1]

Analogous to the preparation of Example 996B, reaction of Example 1042G (150 mg, (0.22 mmol), formaldehyde (6.50 mg, 0.22 mmol), acetic acid (0.12 ml, 0.22 mmol) and sodium cyanoborohydride (13.61 mg, 0.22 mmol) in MeOH (2 mL) yielded Example 1042 (1.50 mg, 2% yield) as a white solid. LC-MS Method E: RT=1.35 min, [M+H]⁺=593.3; LC-MS Method F: RT=0.96 min, [M+H]⁺=593.3. 1H NMR (400 MHz, DMSO-d6) δ=8.53 (br s, 1H), 8.01 (s, 1H), 7.42 (s, 1H), 7.17 (s, 1H), 6.81 (br s, 1H), 6.65 (s, 1H), 6.51 (s, 1H), 5.40 (d, J=1.0 Hz, 2H), 4.85 (br s, 1H), 4.19 (br s, 1H), 3.17 (s, 1H), 2.87 (br s, 3H), 2.61 (s, 3H), 2.14 (br s, 6H), 1.84 (br s, 5H), 1.59 (br s, 2H), 1.46-1.32 (m, 1H), 1.24 (s, 1H).

### Example 1043

### Preparation of N—((R)-1-(4-amino-7-(5-carbamoyl-6-methoxypyridin-2-yl) pyrrolo[2,1-f] [1,2,4] triazin-5-yl) piperidin-3-yl)-2-methyl-4-((4-((S)-2-methylpyrrolidin-1-yl) cyclohexyl) oxy) thiazole-5-carboxamide

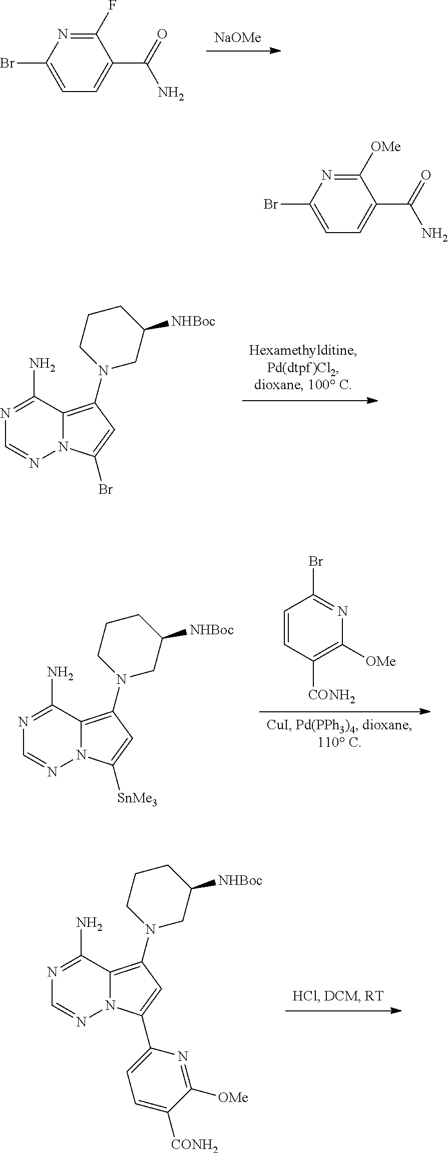

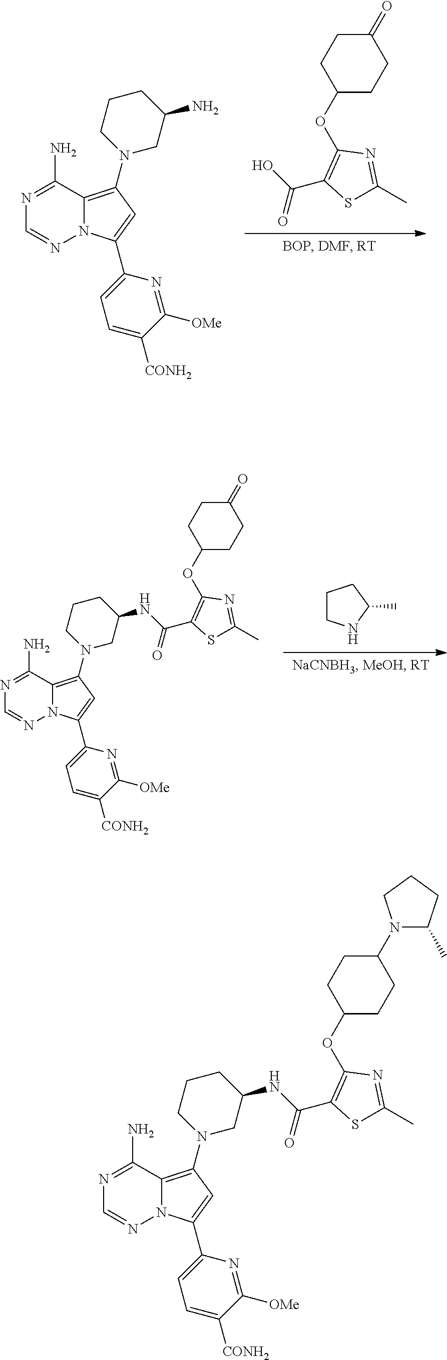

### Example 1043A. Preparation of 6-bromo-2-methoxynicotinamide

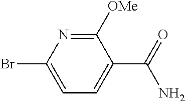 [SMILES: COc1nc(Br)ccc1C(N)=O]

To a stirred solution of 6-bromo-2-fluoronicotinamide (1.7 g, 7.76 mmol) in MeOH (2 mL) was added sodium methoxide (1.49 g, 7.76 mmol) at room temperature. After being stirred for 4 h, the reaction was quenched with 1.5M HCl solution, concentrated and partitioned between water and ethyl acetate. The organic layer was separated, dried over anhydrous sodium sulphate and concentrated under reduced pressure to afford Example 1043A (1.5 g, 84% yield) as pale-yellow solid. MS: [M+H]⁺=231.3.

### Example 10431B: Preparation of tert-butyl (R)-(1-(4-amino-7-(trimethylstannyl) pyrrolo[2,1-f][1,2,4] triazin-5-yl) piperidin-3-yl) carbamate

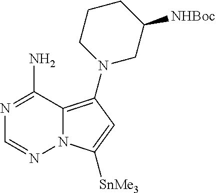

To a stirred solution of tert-butyl (R)-(1-(4-amino-7-bromopyrrolo[2,1-f][1,2,4]triazin-5-yl) piperidin-3-yl) carbamate (3 g, 7.29 mmol) and hexamethylditin (2.8 g, 8.7 mmol) in 1,4-dioxane (20 mL), was added [1,1′-bis(di-tert-butylphosphino) ferrocene]dichloropalladium(II) (0.47 g, 0.73 mmol) at room temperature. The reaction mixture was purged with argon and then heated to 100° C. After being stirred for 3 h, the suspension was filtered through celite bed and the bed was washed with ethyl acetate. The filtrate was concentrated under reduced pressure to afford the crude product. The residue was purified using CombiFlash (celite slurry, 80 g column 40-50% ethyl acetate in pet. ether as eluent) to afford title compound Example 1043B (2.1 g, 68% yield) as brown oil. MS: [M+H]⁺=497.2.

### Example 1043C. Preparation of tert-butyl (R)-(1-(4-amino-7-(5-carbamoyl-6-ethoxypyridin-2-1) pyrrolo[2,1-f] [1,2,4]triazin-5-yl) piperidin-3-yl) carbamate

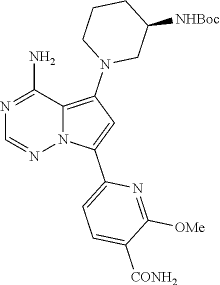 [SMILES: COc1nc(-c2cc(N3CCC[C@@H](NC(=O)OC(C)(C)C)C3)c3c(N)ncnn23)ccc1C(N)=O]

Analogous to the preparation of Example 1026D, reaction of Example 1043B (2.1 g, 4.24 mmol), Copper(I) iodide (0.081 g, 0.42 mmol), Example 1043A (1.96 g, 8.48 mmol) and tetrakis(triphenylphosphine)palladium (0.49 g, 0.42 mmol) in dioxane (10 mL) at 100° C. for 16 h afforded crude Example 1043C (1 g, 48% yield) as brown semi solid. MS: [M+H]⁺=483.3.

### Example 1043D: Preparation of (R)-6-(4-amino-5-(3-aminopiperidin-1-yl) pyrrolo[2,1-f] [1,2,4]triazin-7-yl)-2-methoxynicotinamide. HCl

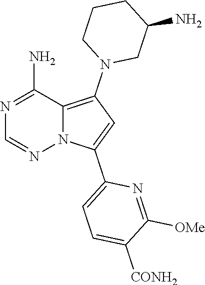 [SMILES: COc1nc(-c2cc(N3CCC[C@@H](N)C3)c3c(N)ncnn23)ccc1C(N)=O]

Analogous to the preparation of Example 996G, reaction of Example 1043C (750 mg, 0.83 mmol) with 4M hydrogen chloride in 1,4 dioxane (181 mg, 4.97 mmol) in DCM (3 mL) at rt for 4 h afforded Example 1043D (500 mg, 84% yield) as a yellow solid. MS: [M+H]⁺=383.1.

### Example 1043E: Preparation of (R)—N-(1-(4-amino-7-(5-carbamoyl-6-methoxypyridin-2-yl) pyrrolo[2,1-f][1,2,4]triazin-5-yl) piperidin-3-yl)-2-methyl-4-((4-oxocyclohexyl) oxy) thiazole-5-carboxamide

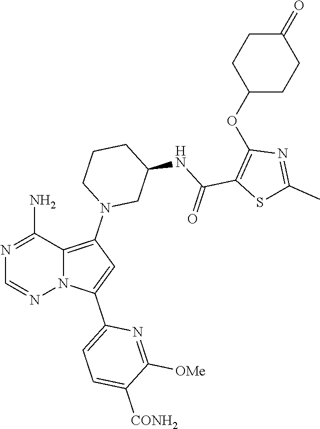 [SMILES: COc1nc(-c2cc(N3CCC[C@@H](NC(=O)c4sc(C)nc4OC4CCC(=O)CC4)C3)c3c(N)ncnn23)ccc1C(N)=O]

Analogous to the preparation of Example 996, reaction of Example 1043D (500 mg, 0.65 mmol), Example 1011A (194 mg, 0.65 mmol), DIPEA (0.45 mL, 2.61 mmol) and BOP (434 mg, 0.98 mmol) in DMF (5 mL) at rt for 4 h afforded the crude Example 1043E (650 mg, 80% yield) as a dark yellow solid. MS: [M+H]⁻=618.2.

### Example 1043

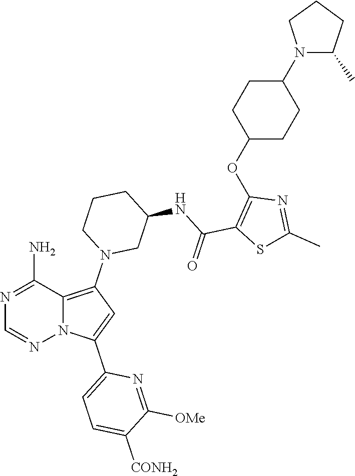 [SMILES: COc1nc(-c2cc(N3CCC[C@@H](NC(=O)c4sc(C)nc4OC4CCC(N5CCC[C@@H]5C)CC4)C3)c3c(N)ncnn23)ccc1C(N)=O]

Analogous to the preparation of Example 996B, reaction of the Example 1043E (100 mg, 0.16 mmol), (2S)-2-methylpyrrolidine (20.61 mg, 0.24 mmol) and sodium cyanoborohydride (60.8 mg, 0.97 mmol)) in MeOH (2 mL) at rt 2 h afforded the crude which was purified by preparative HPLC to afford Example 1043 (11.5 mg, 10% yield). LC-MS Method E: RT=1.39 min, [M+H]⁺=689.3. 1H NMR (400 MHz, DMSO-d₆) δ=8.44-8.38 (m, 1H), 8.30 (d, J=8.0 Hz, 1H), 8.19 (br s, 1H), 7.99 (s, 1H), 7.68 (br d, J=4.3 Hz, 2H), 7.35 (d, J=16.3 Hz, 1H), 6.91 (br s, 1H), 5.20-4.74 (m, 1H), 4.17 (br s, 1H), 4.09 (d, J=3.5 Hz, 3H), 3.2 (m, 7H), 2.99 (br s, 3H), 2.61 (d, J=2.8 Hz, 3H), 2.21 (br s, 2H), 2.00-1.40 (m, 12H), 1.24 (s, 1H), 1.03 (br s, 2H).

### Example 1044

### Preparation of N—((R)-1-(4-amino-7-(5-carbamoyl-6-methoxypyridin-2-yl)pyrrolo[2,1-f][1,2,4]triazin-5-yl)piperidin-3-yl)-2-methyl-4-((4-((R)-2-methylpyrrolidin-1-yl)cyclohexyl)oxy)thiazole-5-carboxamide

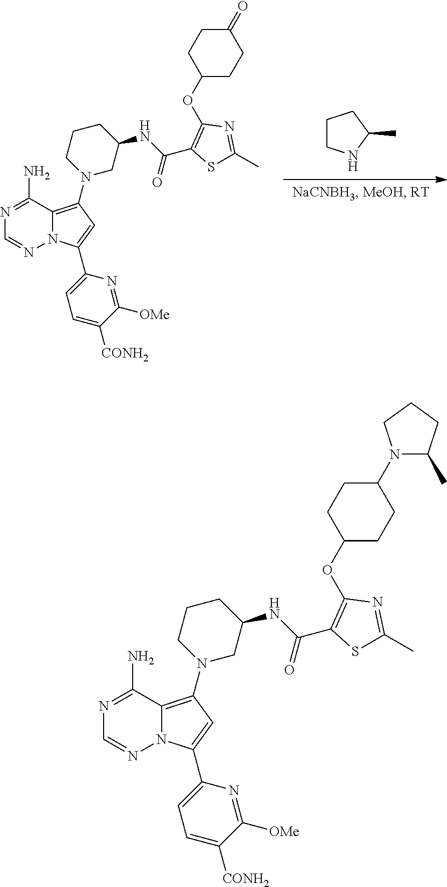

Analogous to the preparation of Example 996B, reaction of Example 1043E (100 mg, 0.16 mmol), (2R)-2-methylpyrrolidine (20.61 mg, 0.24 mmol) and sodium cyanoborohydride (60.8 mg, 0.97 mmol) in MeOH (3 mL) at rt for 2 h afforded the crude which was purified by preparative HPLC to afford Example 1044 (16.5 mg, 15% yield). LC-MS Method E: RT=1.36 min, [M+H]⁺=689.3. 1H NMR (400 MHz, DMSO-d₆) δ=8.47-8.37 (m, 1H), 8.30 (dd, J=2.4, 7.9 Hz, 1H), 8.20 (br s, 1H), 7.99 (d, J=3.0 Hz, 1H), 7.68 (br d, J=5.8 Hz, 2H), 7.35 (d, J=14.5 Hz, 1H), 6.90 (br s, 1H), 5.23-4.70 (m, 1H), 4.22 (br s, 1H), 4.09 (d, J=2.5 Hz, 3H), 3.22 (m, 6H), 2.99 (br s, 2H), 2.65-2.57 (m, 3H), 2.30-2.05 (m, 2H), 2.00-1.73 (m, 4H), 1.72-1.27 (m, 14H).

### Example 1045

### Preparation of (R)—N-(1-(4-amino-7-(5-carbamoyl-6-methoxypyridin-2-yl)pyrrolo[2,1-f][1,2,4]triazin-5-yl)piperidin-3-yl)-4-((4-(azetidin-1-yl)cyclohexyl)oxy)-2-methylthiazole-5-carboxamide

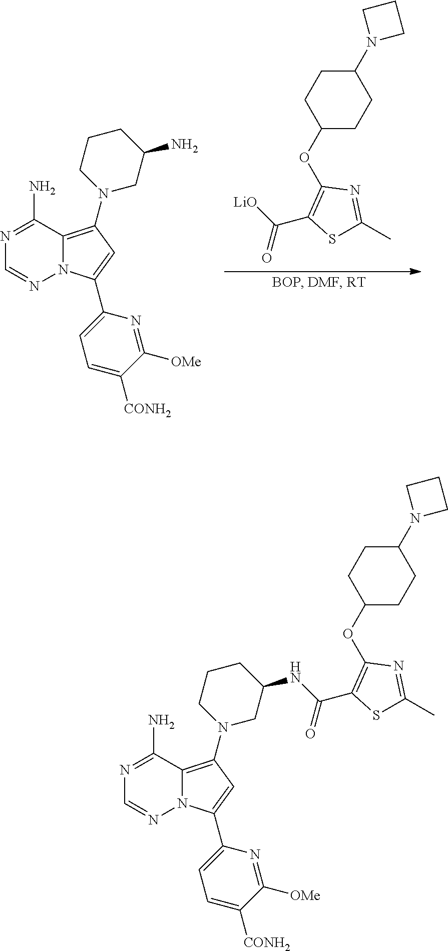

Analogous to the preparation of Example 996, reaction of the Example 1043D (250 mg, 0.65 mmol), Example 1037C (194 mg, 0.65 mmol), BOP (434 mg, 0.98 mmol) and DIPEA (0.45 mL, 2.61 mmol) in DMF (5 mL) at rt 4 h afforded the crude which was purified by preparative HPLC to afford Example 1045 (1046 mg, 11% yield). MS Method E: RT=1.67 min, [M+H]⁺=661.3; ¹H NMR (400 MHz, DMSO-d₆) δ ppm 7.87 (d, J=1.9 Hz, 2H), 7.33 (br s, 1H), 6.85-6.61 (m, 3H), 5.13-4.95 (m, 1H), 4.69 (br d, J=1.8 Hz, 1H), 4.48 (br d, J=8.1 Hz, 2H), 4.27 (br d, J=2.3 Hz, 1H), 4.21-4.04 (m, 1H), 3.13 (br s, 1H), 2.90 (br s, 4H), 2.61 (s, 3H), 2.20-1.98 (m, 5H), 1.95-1.72 (m, 4H), 1.60 (br s, 2H), 1.21-0.79 (m, 9H).

### Example 1046

### Preparation of N—((R)-1-(4-amino-7-(5-carbamoyl-6-ethoxypyridin-2-yl) pyrrolo[2,1-f] [1,2,4] triazin-5-yl) piperidin-3-yl)-2-methyl-4-((1S,4S)-4-(pentan-3-ylamino)

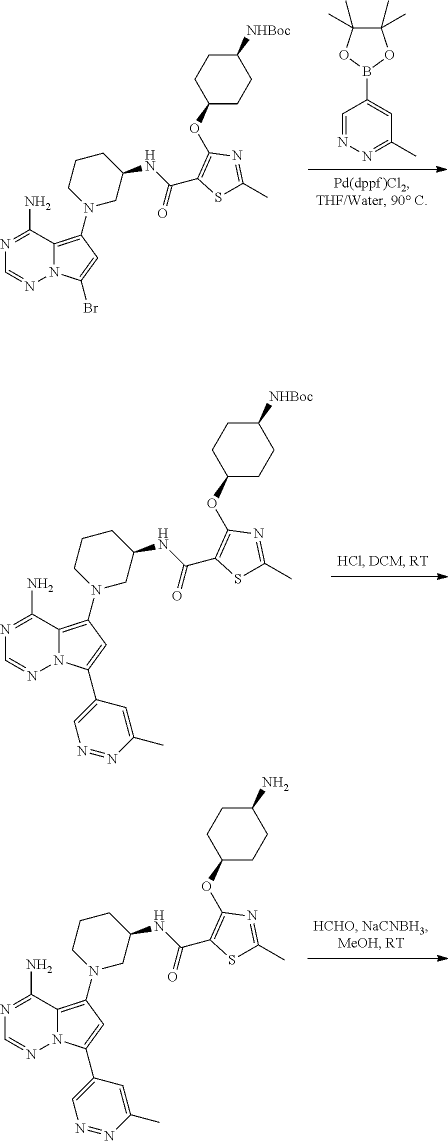

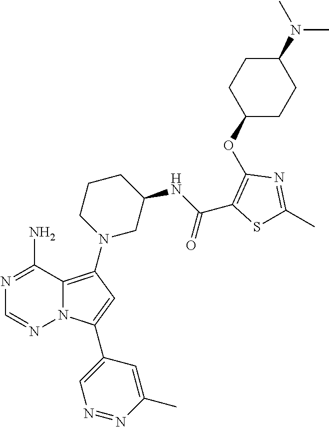 [SMILES: Cc1cc(-c2cc(N3CCC[C@@H](NC(=O)c4sc(C)nc4O[C@H]4CC[C@@H](N(C)C)CC4)C3)c3c(N)ncnn23)cnn1]

### Example 1046A. Preparation of tert-butyl ((1S,4S)-4-((5-(((R)-1-(4-amino-7-(6-methylpyridazin-4-yl) pyrrolo[2,1-f][1,2,4] triazin-5-yl) piperidin-3-yl) carbamoyl)-2-methylthiazol-4-yl) oxy) cyclohexyl) carbamate

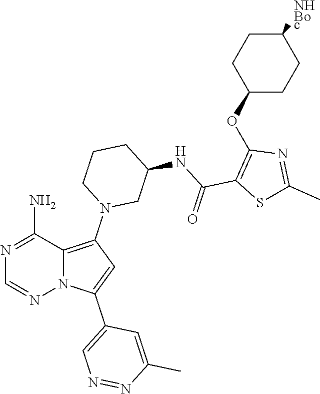 [SMILES: Cc1cc(-c2cc(N3CCC[C@@H](NC(=O)c4sc(C)nc4O[C@H]4CC[C@@H](NC(=O)OC(C)(C)C)CC4)C3)c3c(N)ncnn23)cnn1]

Analogous to the preparation of Example 996F, reaction of Example 1002C (500 mg, 0.77 mmol), 3-methyl-5-(4,4,5,5-tetramethyl-1,3,2-dioxaborolan-2-yl) pyridazine (678 mg, 3.08 mmol) and potassium phosphate, tribasic, (408 mg, 1.92 mmol) and [1,1-bis(diphenylphosphino)ferrocene]dichloropalladium(II) (113 mg, 0.15 mmol) in THF (5 mL)/H₂O (0.55 mL) at 90° C. in microwave for 2 h afforded the crude Example 1046A (450 mg, 88% yield) MS: [M+H]⁺=663.6.

### Example 1046B: Preparation of N—((R)-1-(4-amino-7-(6-methylpyridazin-4-yl) pyrrolo[2,1-f] [1,2,4] triazin-5-yl) piperidin-3-yl)-4-(((1S,4S)-4-aminocyclohexyl) oxy)-2-methylthiazole-5-carboxamide. HCl

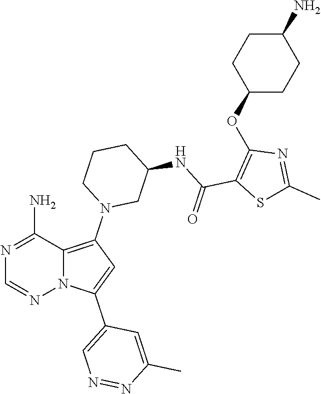 [SMILES: Cc1cc(-c2cc(N3CCC[C@@H](NC(=O)c4sc(C)nc4O[C@H]4CC[C@@H](N)CC4)C3)c3c(N)ncnn23)cnn1]

Analogous to the preparation of Example 996G, reaction of Example 1046A (450 mg, 0.68 mmol) with 4M HCl in 1,4 dioxane (149 mg, 4.07 mmol) in DCM (3 mL) at rt for 4 h afforded crude Example 1046B (340 g, 89% yield) as a yellow solid. MS: [M+H]⁺=563.4.

### Example 1046

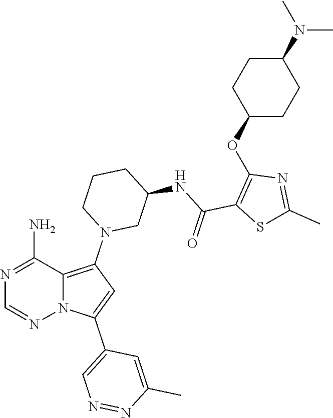 [SMILES: Cc1cc(-c2cc(N3CCC[C@@H](NC(=O)c4sc(C)nc4O[C@H]4CC[C@@H](N(C)C)CC4)C3)c3c(N)ncnn23)cnn1]

Analogous to the preparation of Example 996, reaction of the Example 1046B (200 mg, 0.35 mmol), formaldehyde (10.67 mg, 0.35 mmol) and sodium cyanoborohydride (134 mg, 2.13 mmol) in MeOH (3 mL) at rt for 4 h afforded the crude which was purified by preparative HPLC to yield Example 1046 (12 mg, 6% yield). LC-MS Method E: RT=1.29 min, [M+H]⁺=591.2; ¹H NMR (400 MHz, DMSO-d₆) δ ppm 7.87 (d, J=1.9 Hz, 2H), 7.33 (br s, 1H), 6.85-6.61 (m, 3H), 5.13-4.95 (m, 1H), 4.69 (br d, J=1.8 Hz, 1H), 4.48 (br d, J=8.1 Hz, 2H), 4.27 (br d, J=2.3 Hz, 1H), 4.21-4.04 (m, 1H), 3.13 (br s, 1H), 2.90 (br s, 4H), 2.61 (s, 3H), 2.20-1.98 (m, 5H), 1.95-1.72 (m, 4H), 1.60 (br s, 2H), 1.21-0.79 (m, 9H).

### Example 1047

### Preparation of N—((R)-1-(4-amino-7-(6-methylpyridazin-4-yl)pyrrolo[2,1-f][1,2,4]triazin-5-yl)piperidin-3-yl)-4-((4-(azetidin-1-yl)cyclohexyl)oxy)-2-methylthiazole-5-carboxamide

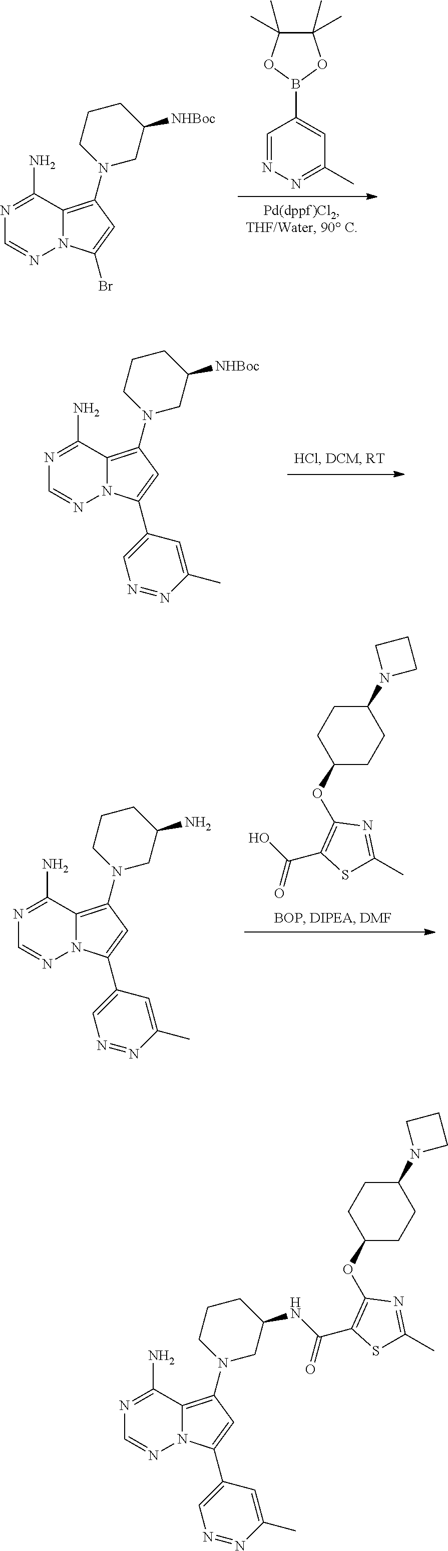

### Example 1047A. Preparation of tert-butyl (R)-(1-(4-amino-7-(6-methylpyridazin-4-yl)pyrrolo[2,1-f][1,2,4]triazin-5-yl)piperidin-3-yl)carbamate

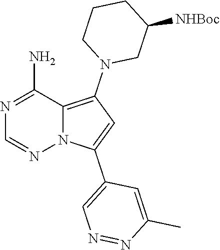 [SMILES: Cc1cc(-c2cc(N3CCC[C@@H](NC(=O)OC(C)(C)C)C3)c3c(N)ncnn23)cnn1]

Analogous to the preparation of Example 996F, reaction of the tert-butyl (R)-(1-(4-amino-7-bromopyrrolo[2,1-f][1,2,4]triazin-5-yl) piperidin-3-yl) carbamate (400 mg, 0.97 mmol), 3-methyl-5-(4,4,5,5-tetramethyl-1,3,2-dioxaborolan-2-yl) pyridazine (642 mg, 2.92 mmol), potassium phosphate, tribasic, 97%, pure, anhydrous (516 mg, 2.43 mmol) and 1,1′-bis(di-tert-butylphosphino)ferrocene-palladium dichloride (63.4 mg, 0.1 mmol) in dioxane (5 mL)/water (0.556 mL) at 90° C. for 3 h afforded the crude Example 1047A (164 mg, 40% yield) which was taken to the next step without further purification. MS: [M+H]⁺=423.2.

### Example 1047B. Preparation of (R)-5-(3-aminopiperidin-1-yl)-7-(6-methylpyridazin-4-yl)pyrrolo[2,1-f][1,2,4]triazin-4-amine. HCl

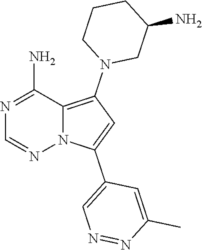 [SMILES: Cc1cc(-c2cc(N3CCC[C@@H](N)C3)c3c(N)ncnn23)cnn1]

Analogous to the preparation of Example 996G, reaction of Example 1047A (150 mg, 0.35 mmol) and HCl in 1,4 dioxane (77 mg, 2.12 mmol) in DCM (3 mL) for 4 h afforded Example 1047B (118 mg, 95% yield) which was used in the next step without further purification. MS: [M+H]⁺=323.2.

### Example 1047

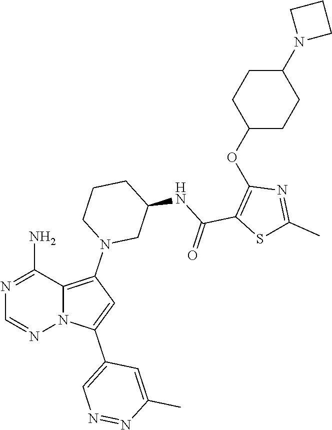 [SMILES: Cc1cc(-c2cc(N3CCC[C@@H](NC(=O)c4sc(C)nc4OC4CCC(N5CCC5)CC4)C3)c3c(N)ncnn23)cnn1]

Analogous to the preparation of Example 996, reaction of Example 1047B (100 mg, 0.31 mmol), Example 1037C (92 mg, 0.46 mmol), BOP (273 mg, 0.62 mmol) and DIPEA (0.05 mL, 0.31 mmol) in DMF (1.0 mL) at rt for 4 h afforded the crude which was purified by preparative HPLC to yield Example 1047 (12 mg, 6% yield) as an off-white solid. LC-MS Method E: RT=1.33 min, [M+H]⁺=603.3; ¹H NMR (400 MHz, DMSO-d₆) δ=8.47-8.37 (m, 1H), 8.30 (dd, J=2.4, 7.9 Hz, 1H), 8.20 (br s, 1H), 7.99 (d, J=3.0 Hz, 1H), 7.68 (br d, J=5.8 Hz, 2H), 7.35 (d, J=14.5 Hz, 1H), 6.90 (br s, 1H), 5.23-4.70 (m, 1H), 4.22 (br s, 1H), 4.09 (d, J=2.5 Hz, 3H), 3.22 (m, 6H), 2.99 (br s, 2H), 2.65-2.57 (m, 3H), 2.30-2.05 (m, 2H), 2.00-1.73 (m, 4H), 1.72-1.27 (m, 14H).

### Example 1048

### Preparation of (R)—N-(1-(4-amino-7-cyanopyrrolo[2,1-f][1,2,4] triazin-5-yl) piperidin-3-yl)-4-((4-(3-hydroxyazetidin-1-yl) cyclohexyl) oxy)-2-methylthiazole-5-carboxamide

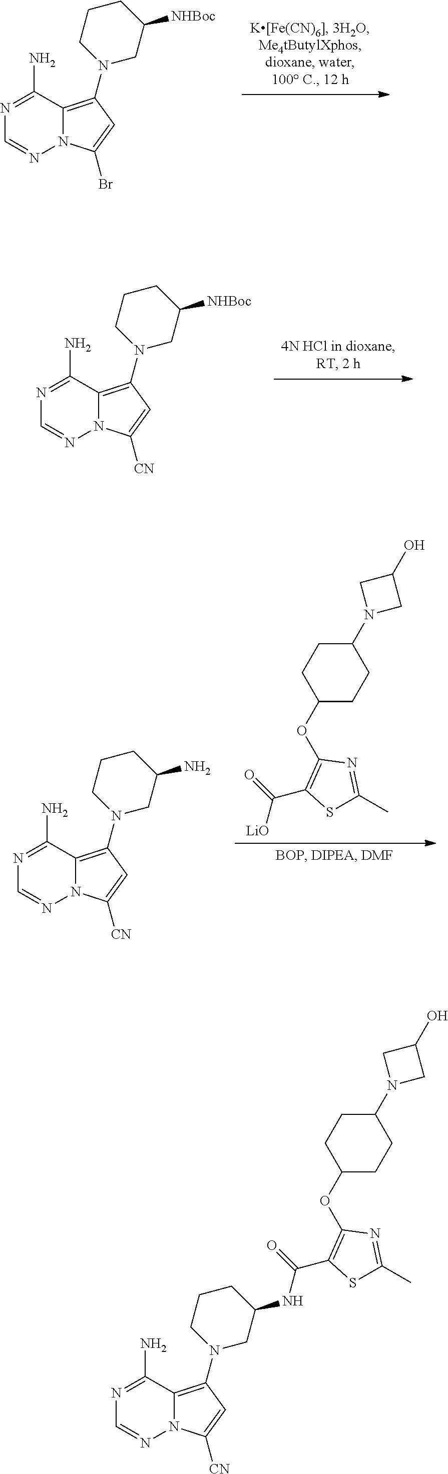

### Example 1048A, Intermediate 29: Preparation of tert-butyl (R)-(1-(4-amino-7-cyanopyrrolo[2,1-f][1,2,4]triazin-5-yl) piperidin-3-yl) carbamate

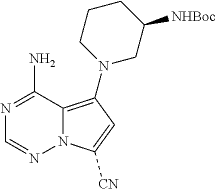

A mixture of tert-butyl (R)-(1-(4-amino-7-bromopyrrolo[2,1-f][1,2,4]triazin-5-yl) piperidin-3-yl) carbamate (2 g, 4.86 mmol), potassium hex cyanoferrate(II) trihydrate (2.05 g, 4.86 mmol) and di-tert-butyl(2′,4′,6′-triisopropyl-[1,1′-biphenyl]-2-yl) phosphine (0.413 g, 0.973 mmol) in 1,4-dioxane (5 mL) and water (5 mL) was degassed by purging with argon for 5 min. ^{t}BuXPhos-Pd-G3 (0.86 g, 0.97 mmol) was added to the mixture and heated at 100° C. 12 h. The reaction mixture was filtered through celite, diluted with ethyl acetate (50 mL), washed with water (3×20 mL) followed by brine (1×25 mL) and concentrated in vacuo to get the crude. The crude product was purified by flash chromatography (2-10% MeOH in DCM as eluent) to Example 1048A (1.5 g, 86% yield) as a light brown solid. LC-MS Method J: RT=1.47 min, [M+H]⁺=358.3.

### Example 1048B: Preparation of (R)-4-amino-5-(3-aminopiperidin-1-yl) pyrrolo[2,1-f][1,2,4]triazine-7-carbonitrile. HCl

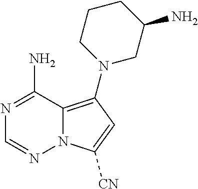

Analogous to the preparation of Example 996G, reaction of Example 1048A (1.4 g, 3.92 mmol) with 4N HCl in dioxane (9.79 mL, 39.2 mmol) in DCM (3 mL) at rt for 3 h afforded Example 1048B (1.1 g, 96% yield) as a light-yellow solid. LC-MS Method J: RT=0.63 min, [M+H]⁺=258.1.

### Example 1048

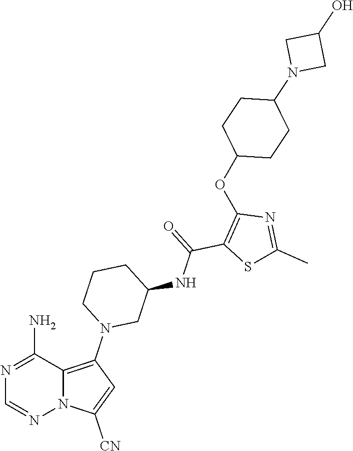 [SMILES: Cc1nc(OC2CCC(N3CC(O)C3)CC2)c(C(=O)N[C@@H]2CCCN(c3cc(C#N)n4ncnc(N)c34)C2)s1]

Analogous to the preparation of Example 996, reaction of Example 1048B (100 mg, 0.34 mmol), 4-((4-(3-hydroxyazetidin-1-yl) cyclohexyl) oxy)-2-methylthiazole-5-carboxylic acid, lithium salt (Iso-1) (109 mg, 0.34 mmol), BOP (226 mg, 0.51 mmol) and DIPEA (0.178 mL, 1.02 mmol) in DMF (2 mL) at rt for 5 h afforded crude which was purified by preparative LCMS/HPLC Method V to afford Example 1048 (66 mg, 35% yield) as an off-white solid. LC-MS Method E: RT=1.27 min, [M+H]⁺=552.2; ¹H NMR (400 MHz, DMSO-d₆) δ=8.69 (br s, 1H), 8.01 (s, 1H), 7.33 (s, 2H), 6.90 (br s, 1H), 5.24 (br s, 1H), 5.06 (br s, 1H), 4.26-4.00 (m, 2H), 3.47 (br dd, J=3.1, 5.9 Hz, 1H), 3.17-3.11 (m, 1H), 2.82 (br s, 2H), 2.60 (s, 5H), 2.08 (s, 1H), 1.95-1.75 (m, 6H), 1.69-1.37 (m, 6H), 1.36-1.26 (m, 2H).

### Example 1049

### Preparation of (R)—N-(1-(4-amino-7-cyanopyrrolo[2,1-f][1,2,4]triazin-5-yl)piperidin-3-yl)-4-((4-(azetidin-1-yl)cyclohexyl)oxy)-2-methylthiazole-5-carboxamide

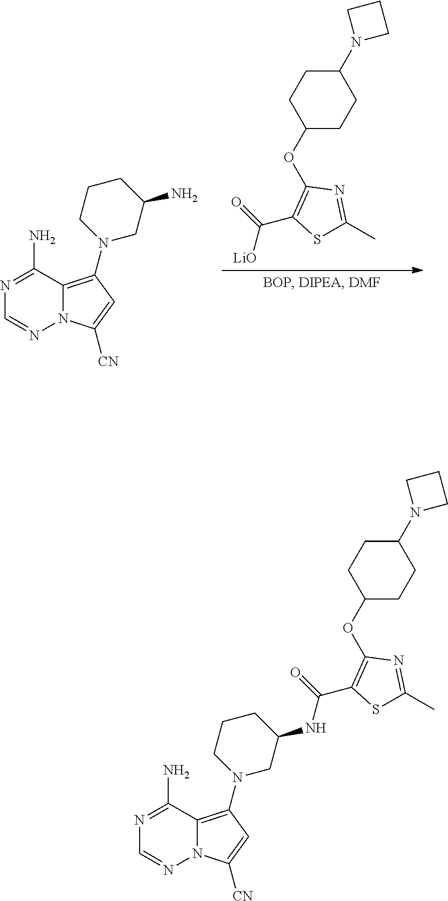

Analogous to the preparation of Example 996, reaction of Example 1048B (120 mg, 0.41 mmol), 4-((4-(azetidin-1-yl)cyclohexyl)oxy)-2-methylthiazole-5-carboxylic acid, lithium salt (149 mg, 0.49 mmol), BOP (271 mg, 0.613 mmol) and DIPEA (0.21 mL, 1.22 mmol) in DMF (2 mL) afforded the crude which was purified by the reverse phase chromatography to afford Example 1049 (64 mg, 29% yield) as an off white solid. LC-MS Method E: RT=1.33 min, [M+H]⁺=632.2. 1H NMR (400 MHz, DMSO-d6) δ=8.68 (br s, 1H), 8.02 (s, 1H), 7.35 (s, 1H), 7.28 (br s, 1H), 6.92 (br s, 1H), 5.05 (br s, 1H), 4.19 (br s, 1H), 3.18 (br d, J=8.0 Hz, 1H), 2.99-2.69 (m, 3H), 2.60 (s, 3H), 2.05-1.76 (m, 8H), 1.71-1.38 (m, 5H), 1.35-1.21 (m, 2H)

### Example 1050

### Preparation of (R)-4-(4-amino-5-(3-(4-((4-(azetidin-1-yl)cyclohexyl)oxy)-2-methylthiazole-5-carboxamido)piperidin-1-yl)pyrrolo[2,1-f][1,2,4]triazin-7-yl)thiazole-2-carboxamide

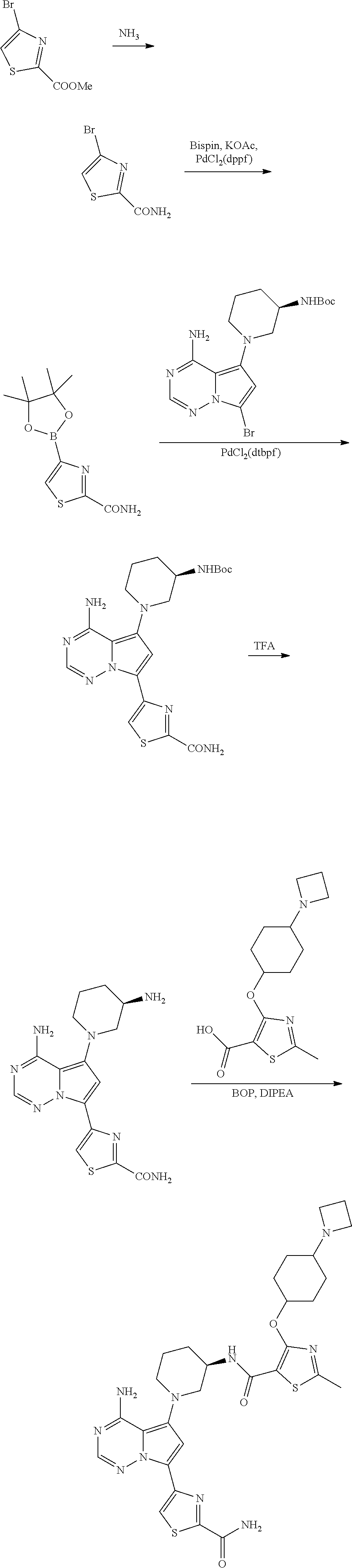

### Example 1050A. Preparation of 4-(4,4,5,5-tetramethyl-1,3,2-dioxaborolan-2-yl)thiazole-2-carboxamide

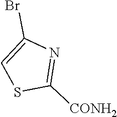

A mixture of methyl 4-bromothiazole-2-carboxylate (500 mg, 2.25 mmol) and 7N ammonia solution in methanol (7.1 mL, 49.5 mmol) was taken in a sealed tub- and was stirred at rt for 12 h. The reaction mixture was evaporated under reduced pressure to afford Example 1050A (460 mg, 96% yield) pale yellow solid. MS: [M+H]⁺=329.2.

### Example 1050B: Preparation of 4-(4,4,5,5-tetramethyl-1,3,2-dioxaborolan-2-yl)thiazole-2-carboxamide

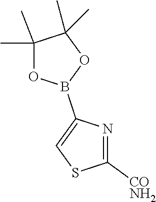 [SMILES: CC1(C)OB(c2csc(C(N)=O)n2)OC1(C)C]

Analogous to the preparation of Example 996E, reaction of the Example 1050A (3 g, 14.49 mmol), bis(pinacolato)diboron (4.78 g, 18.84 mmol) potassium acetate (2.84 g, 29.0 mmol) and PdCl₂(dppf)·DCM (1.183 g, 1.45 mmol) in dioxane (50 mL) at 90° C. for 6 h afforded crude Example 1050B (3.5 g, 95% yield) as a brown solid which was taken to next step without further purification. LCMS Method E: MS [M+H]⁺=173.0 (boronic acid).

### Example 1050C: Preparation of tert-butyl (R)-(1-(4-amino-7-(2-carbamoylthiazol-4-yl)pyrrolo[2,1-f][1,2,4]triazin-5 yl)piperidin-3-yl)carbamate

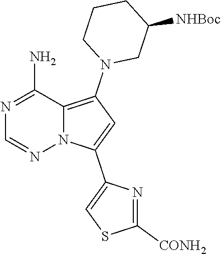 [SMILES: CC(C)(C)OC(=O)N[C@@H]1CCCN(c2cc(-c3csc(C(N)=O)n3)n3ncnc(N)c23)C1]

Analogous to the preparation of Example 996F, reaction of tert-butyl (R)-(1-(4-amino-7-bromopyrrolo[2,1-f][1,2,4]triazin-5-yl)piperidin-3-yl)carbamate (3 g, 7.29 mmol), Example 1050B (2.78 g, 10.94 mmol), potassium phosphate tribasic anhydrous (7.29 mL, 2M, 14.59 mmol) and [1,1′-bis(di-tert-butylphosphino)ferrocene]dichloropalladium(II) (0.47 g, 0.73 mmol) in THF (45 mL) at 70° C. for 5 h afforded Example 1050C (3 g, 90% yield) as brown solid. MS: [M+H]⁺=459.2.

### Example 1050D. Preparation of (R)-4-(4-amino-5-(3-aminopiperidin-1-yl)pyrrolo[2,1-f][1,2,4]triazin-7-yl)thiazole-2-carboxamide. HCl

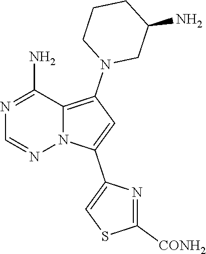 [SMILES: NC(=O)c1nc(-c2cc(N3CCC[C@@H](N)C3)c3c(N)ncnn23)cs1]

Analogous to the preparation of Example 1002E, reaction of the Example 1050C (2 g, 4.36 mmol) and HCl in 1,4-dioxane (10.9 mL, 4M, 43.6 mmol) in 1,4-dioxane (20 mL) at rt for 2 h afforded Example 1050D (1.5 g, 96% yield) as yellow solid. MS: [M+H]⁺=359.1.

### Example 1050

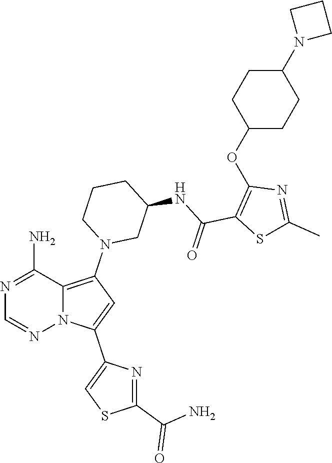 [SMILES: Cc1nc(OC2CCC(N3CCC3)CC2)c(C(=O)N[C@@H]2CCCN(c3cc(-c4csc(C(N)=O)n4)n4ncnc(N)c34)C2)s1]

Analogous to the preparation of Example 996, reaction Example 1050D (40 mg, 0.11 mmol), Example 1037C (33.1 mg, 0.11 mmol), BOP (49.4 mg, 0.11 mmol) and DIPEA (0.05 mL, 0.33 mmol) in DMF afforded Example 1050 (19.4 mg, 27% yield) LC-MS Method E: RT=1.26 min, [M+H]⁺=637.2; LC-MS Method F: RT=0.87 min, [M+H]⁺=637.3; 1H NMR δ ppm 8.55 (s, 1H), 8.17 (br s, 1H), 7.97 (s, 1H), 7.94 (s, 1H), 7.32 (br s, 1H), 7.25 (s, 1H), 5.04 (td, J=2.9, 5.4 Hz, 1H), 4.21 (br s, 1H), 2.97 (br s, 5H), 2.60 (s, 4H), 2.20-1.97 (m, 3H), 1.99-1.76 (m, 7H), 1.73-1.38 (m, 5H), 1.37-1.25 (m, 2H).

### Example 1051

### Preparation of 4-(4-amino-5-((R)-3-(4-(((1S,4S)-4-(dimethylamino)cyclohexyl)oxy)-2-methylthiazole-5-carboxamido)piperidin-1-yl)pyrrolo[2,1-f][1,2,4]triazin-7-yl)thiazole-2-carboxamide

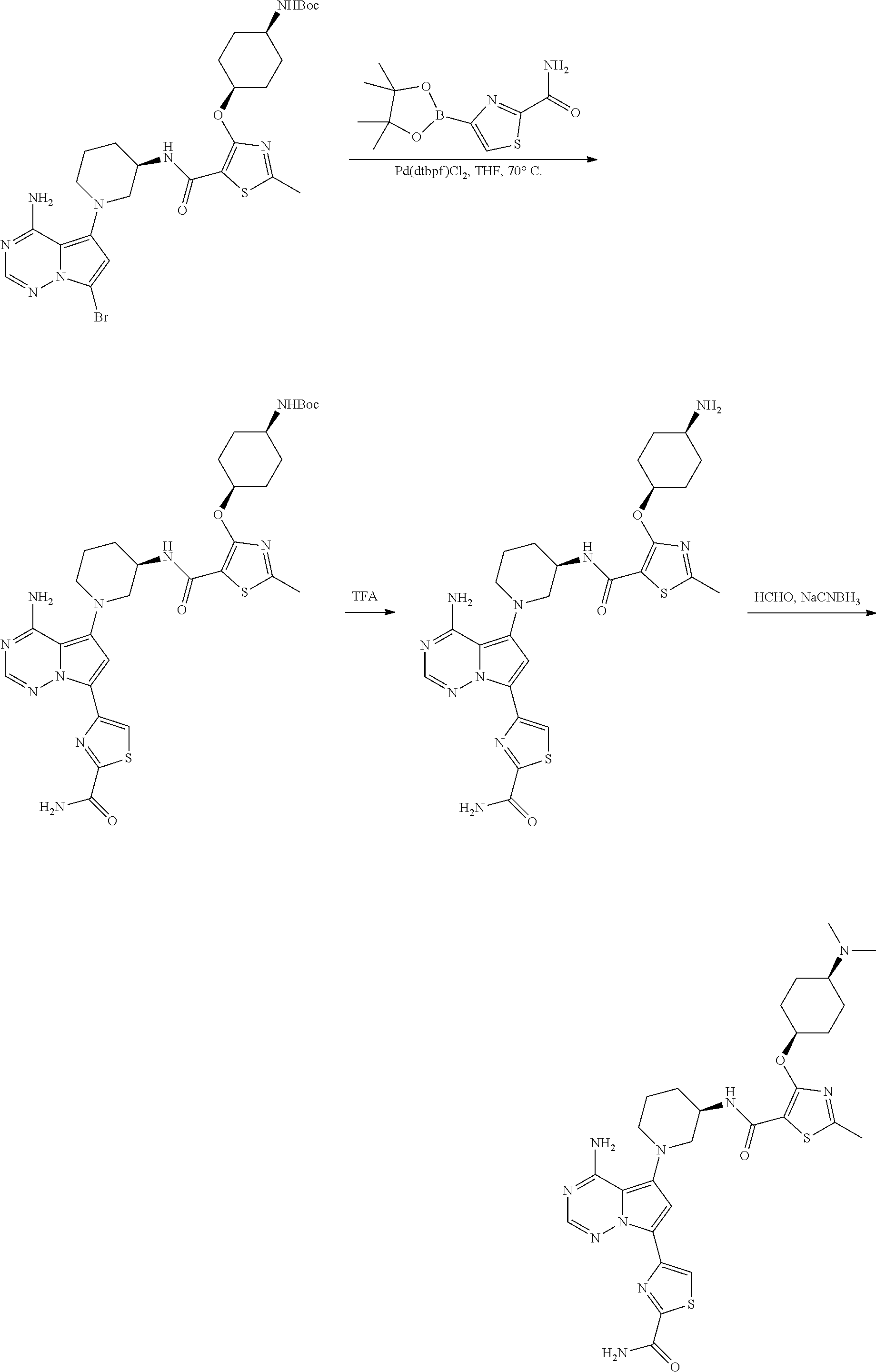

### Example 1051A: Preparation of tert-butyl ((1S,4S)-4-((5-(((R)-1-(4-amino-7-(2-carbamoylthiazol-4-yl)pyrrolo[2,1-f][1,2,4]triazin-5-yl)piperidin-3-yl)carbamoyl)-2-methylthiazol-4-yl)oxy)cyclohexyl)carbamate

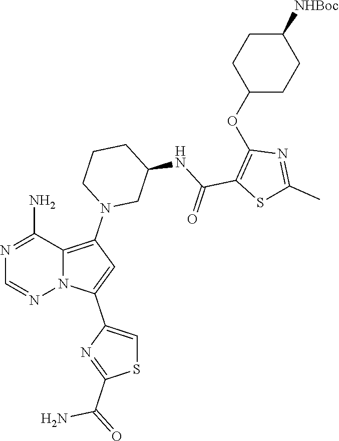

Analogous to the preparation of Example 996F reaction of Example 1002C (300 mg, 0.46 mmol), Example 1050B (235 mg, 0.92 mmol), tripotassium phosphate (0.69 mL, 1.38 mmol) and [1,1′-bis(di-tert-butylphosphino)ferrocene]dichloropalladium(II) (15.05 mg, 0.023 mmol) in THF/water afforded Example 1051A (300 mg, 93% yield) as yellow solid. MS: [M−H]⁺=695.3.

### Example 1051B: Preparation of 4-(4-amino-5-((R)-3-(4-(((1S,4S)-4-aminocyclohexyl)oxy)-2-methylthiazole-5-carboxamido)piperidin-1-yl)pyrrolo[2,1-f][1,2,4]triazin-7-yl)thiazole-2-carboxamide. TFA

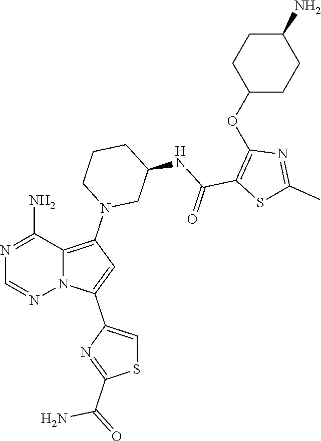

Analogous to the preparation of Example 1002E, reaction of Example 1051A (300 mg, 0.46 mmol) with TFA (0.66 mL, 8.61 mmol) afforded Example 1051B (300 mg, 93% yield) as yellow solid. MS: [M−H]⁺=595.3.

### Example 1051

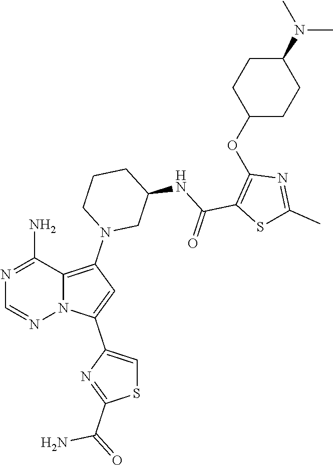

Analogous to the preparation of Example 996, reaction of Example 1051B (50 mg, 0.08 mmol), sodium cyanoborohydride (10.5 mg, 0.17 mmol), acetic acid (0.05 mL, 0.84 mmol) and formaldehyde (2.31 μl, 0.08 mmol) afforded Example 1051 (24.1 mg, 45% yield), LC-MS Method E: RT=1.18 min, [M+H]⁺=625.2; LC-MS Method F: RT=0.85 min, [M+H]⁺=625.2; 1H NMR (400 MHz, DMSO-d6) δ=9.48 (br s, 1H), 8.56 (s, 1H), 8.20 (s, 1H), 7.99 (s, 1H), 7.96 (s, 1H), 7.23 (s, 2H), 5.10 (br s, 1H), 4.17 (br s, 1H), 3.26 (br s, 3H), 3.01 (br s, 1H), 2.85 (br s, 1H), 2.66-2.57 (m, 6H), 2.31-2.18 (m, 5H), 1.87 (br d, J=6.3 Hz, 4H), 1.78-1.47 (m, 6H).

### Example 1052

### Preparation of (R)-2-(4-amino-5-(3-(4-((4-(azetidin-1-yl)cyclohexyl)oxy)-2-methylthiazole-5-carboxamido)piperidin-1-yl)pyrrolo[2,1-f][1,2,4]triazin-7-yl)thiazole-4-carboxamide

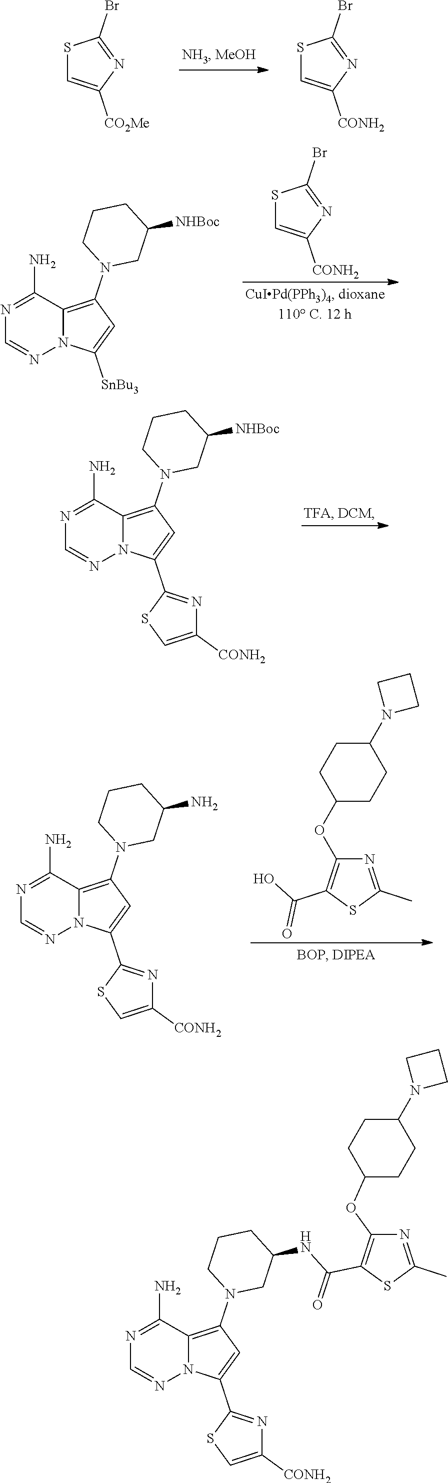

### Example 1052A. Preparation of 2-bromothiazole-4-carboxamide

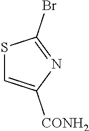

Analogous to the preparation of Example 1050A, reaction of the methyl 2-bromothiazole-4-carboxylate (3 g, 13.51 mmol) with ammonia in methanol (42.5 ml, 7M, 297 mmol) at rt for 12 h afforded crude Example 1053A (2.7 g, 97% yield) as yellow solid. MS [M+H]⁺=207.1.

### Example 1052B: Preparation of tert-butyl (R)-(1-(4-amino-7-(4-carbamoylthiazol-2-yl)pyrrolo[2,1-f][1,2,4]triazin-5-yl)piperidin-3-yl)carbamate

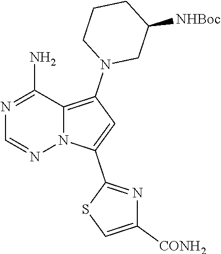 [SMILES: CC(C)(C)OC(=O)N[C@@H]1CCCN(c2cc(-c3nc(C(N)=O)cs3)n3ncnc(N)c23)C1]

Analogous to the preparation of Example 1026D, reaction of Example 1026C (1.75 g, 2.82 mmol), Example 1052A (5.78 g, 2.82 mmol), copper(I) iodide (0.054 g, 0.282 mmol) and Pd(PPh₃)₄ (0.32 g, 0.28 mmol) at 110° C. for 12 h afforded Example 1052B (250 mg, 19% yield) as a yellow solid. MS [M+H]⁺=459.2.

### Example 1052C: Preparation of (R)-2-(4-amino-5-(3-aminopiperidin-1-yl)pyrrolo[2,1-f][1,2,4]triazin-7-yl)thiazole-4-carboxamide. TFA

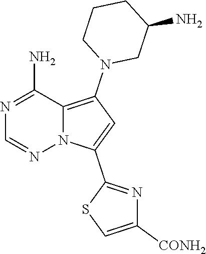 [SMILES: NC(=O)c1csc(-c2cc(N3CCC[C@@H](N)C3)c3c(N)ncnn23)n1]

Analogous to the preparation of Example 1002E, reaction of Example 1051B (250 mg, 0.54 mmol) and TFA (0.42 mL, 5.45 mmol) afforded Example 1052C (190 mg, 90% yield) as a brown solid. MS [M+H]⁺=359.1.

### Example 1052

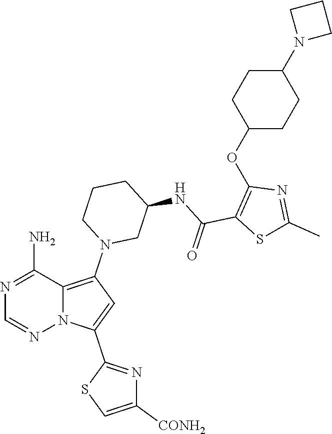 [SMILES: Cc1nc(OC2CCC(N3CCC3)CC2)c(C(=O)N[C@@H]2CCCN(c3cc(-c4nc(C(N)=O)cs4)n4ncnc(N)c34)C2)s1]

Analogous to the preparation of Example 996, reaction of Example 1052C (42.3 mg, 0.12 mmol), Example 1037C (35 mg, 0.12 mmol), BOP (52.2 mg, 0.12 mmol) and DIPEA (0.06 mL, 0.35 mmol) afforded Example 1052 (22.8 mg, 30% yield), LC-MS Method E: RT=1.19 min, [M+H]⁺=637.3; LC-MS Method F: RT=0.88 min, [M+H]⁺=637.2; ¹H NMR (400 MHz, DMSO-d₆) δ ppm 8.24 (s, 1H), 8.09 (s, 1H), 7.79 (br s, 1H), 7.64 (br d, J=0.8 Hz, 1H), 7.37 (s, 2H), 6.82 (s, 1H), 5.04 (br s, 1H), 4.22 (br s, 1H), 3.46-3.33 (m, 5H), 3.26 (br s, 1H), 2.96 (br s, 6H), 2.60 (s, 3H), 2.02-1.77 (m, 6H), 1.70-1.39 (m, 5H), 1.36-1.25 (m, 2H).

### Example 1053

### Preparation of 4-(4-amino-5-((R)-3-(4-((4-(((R)-1-hydroxybutan-2-yl)amino)cyclohexyl)oxy)-2-methylthiazole-5-carboxamido)piperidin-1-yl)pyrrolo[2,1-f][1,2,4]triazin-7-yl)-N-methylthiazole-2-carboxamide

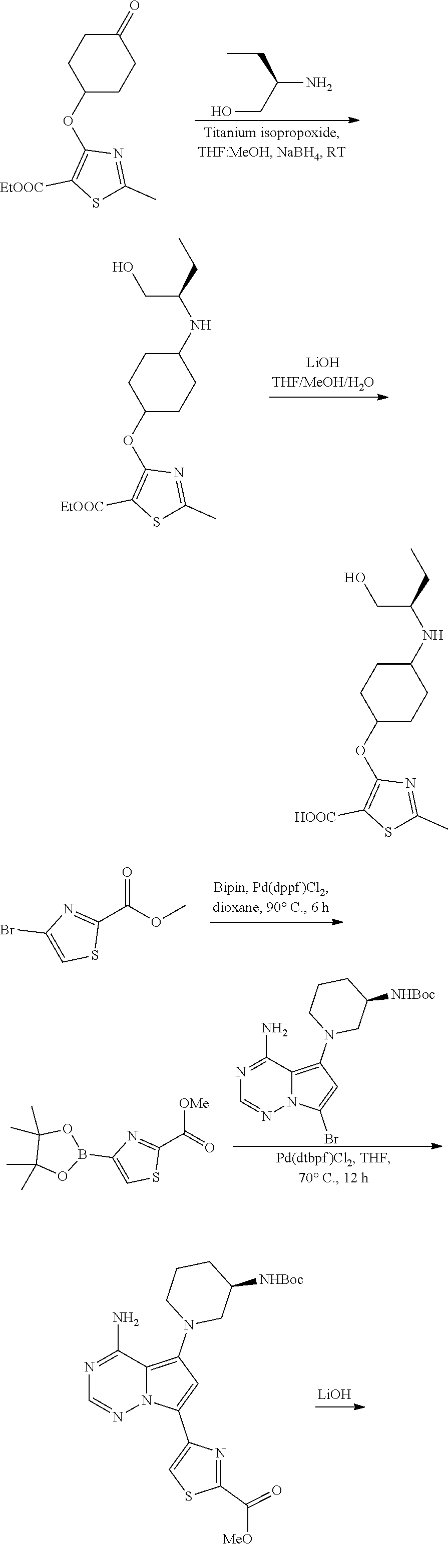

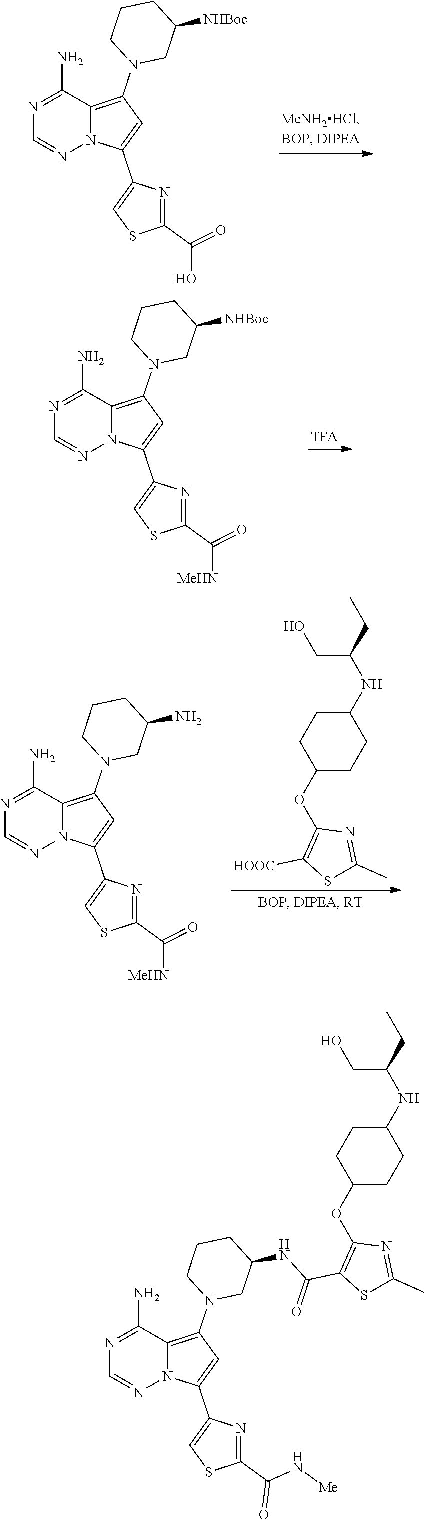

### Example 1053A. Preparation of ethyl (R)-4-((4-((1-hydroxybutan-2-yl)amino)cyclohexyl)oxy)-2-methylthiazole-5-carboxylate

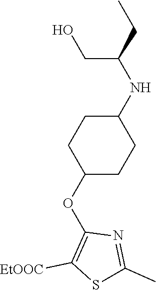 [SMILES: CCOC(=O)c1sc(C)nc1OC1CCC(N[C@H](CC)CO)CC1]

Analogous to the preparation of Example 1000A, reaction of Example 941B (4 g, 14.12 mmol), titanium (IV) isopropoxide (8.02 g, 28.2 mmol), (R)-2-aminobutan-1-ol (7.55 g, 85 mmol) and sodium borohydride (1.06 g, 28.2 mmol) afforded Example 1053A (1.9 g, 38% yield) as brown solid. MS: [M+H]⁺=357.2.

### Example 1053B. Preparation of (R)-4-((4-((1-hydroxybutan-2-yl)amino)cyclohexyl)oxy)-2-methylthiazole-5-carboxylic acid

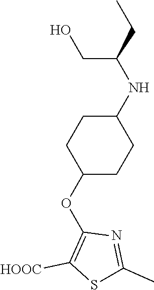 [SMILES: CC[C@H](CO)NC1CCC(Oc2nc(C)sc2C(=O)O)CC1]

Analogous to the preparation of Example 996C, reaction of Example 1053A (1.9 g, 5.33 mmol) and LiOH (0.32 g, 13.32 mmol) afforded Example 1053B (1.4 g, 80% yield) as yellow solid. MS: [M+H]⁺=329.2.

### Example 1053C. Preparation of methyl 4-(4,4,5,5-tetramethyl-1,3,2-dioxaborolan-2-yl)thiazole-2-carboxylate

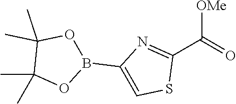 [SMILES: COC(=O)c1nc(B2OC(C)(C)C(C)(C)O2)cs1]

Analogous to the preparation of Example 996E, reaction of methyl 4-bromothiazole-2-carboxylate (3 g, 13.5 mmol), bispin (5.15 g, 20.27 mmol), potassium acetate (2.65 g, 27.0 mmol) and PdCl₂(dppf) (0.99 g, 1.35 mmol) afforded Example 1053C (3.6 g, 99% yield) as a brown paste. MS [M+H]⁺=188.1.

### Example 1053D. Preparation of methyl (R)-4-(4-amino-5-(3-((tert-butoxycarbonyl)amino)piperidin-1-yl)pyrrolo[2,1-f][1,2,4]triazin-7-yl)thiazole-2-carboxylate

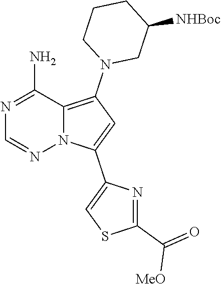 [SMILES: COC(=O)c1nc(-c2cc(N3CCC[C@@H](NC(=O)OC(C)(C)C)C3)c3c(N)ncnn23)cs1]

Analogous to the preparation of Example 996F, reaction of tert-butyl (R)-(1-(4-amino-7-bromopyrrolo[2,1-f][1,2,4]triazin-5-yl)piperidin-3-yl)carbamate (2.5 g, 6.08 mmol), Example 1053C (3.5 g, 13.01 mmol), tripotassium phosphate (9.12 mL, 18.23 mmol, 2M) and [1,1′-bis(di-tert-butylphosphino)ferrocene]dichloropalladium(II) (0.198 g, 0.304 mmol) afforded Example 1053D (2.5 g, 87% yield) as a brown solid. MS [M+H]⁺=474.2.

### Example 1053E. Preparation of (R)-4-(4-amino-5-(3-((tert-butoxycarbonyl)amino)piperidin-1-yl)pyrrolo[2,1-f][1,2,4]triazin-7-yl)thiazole-2-carboxylic acid

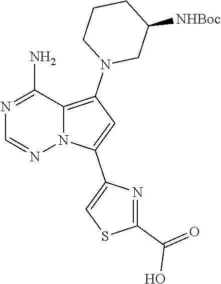 [SMILES: CC(C)(C)OC(=O)N[C@@H]1CCCN(c2cc(-c3csc(C(=O)O)n3)n3ncnc(N)c23)C1]

Analogous to the preparation of Example 996C, reaction of Example 1053D (2.5 g, 5.28 mmol) and LiOH (0.316 g, 13.20 mmol) afforded Example 1053E (2.3 g, 95% yield) as a brown solid. MS [M+H]⁺=460.2.

### Example 1053F. Preparation of tert-butyl (R)-(1-(4-amino-7-(2-(methylcarbamoyl)thiazol-4-yl)pyrrolo[2,1-f][1,2,4]triazin-5-yl)piperidin-3-yl)carbamate

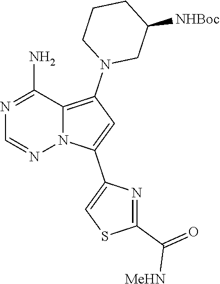 [SMILES: CNC(=O)c1nc(-c2cc(N3CCC[C@@H](NC(=O)OC(C)(C)C)C3)c3c(N)ncnn23)cs1]

Analogous to the preparation of Example 996, reaction of Example 1053E (2.1 g, 4.57 mmol), methylamine hydrochloride (0.617 g, 9.14 mmol), BOP (2.02 g, 4.57 mmol) and DIPEA (2.39 mL, 13.71 mmol) afforded Example 1053F (2.0 g, 91% yield) as yellow solid. MS [M+H]⁺=473.2.

### Example 1053G. Preparation of (R)-4-(4-amino-5-(3-aminopiperidin-1-yl)pyrrolo[2,1-f][1,2,4]triazin-7-yl)-N-methylthiazole-2-carboxamide. TFA

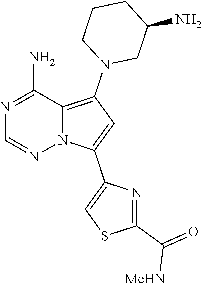 [SMILES: CNC(=O)c1nc(-c2cc(N3CCC[C@@H](N)C3)c3c(N)ncnn23)cs1]

Analogous to the preparation of Example 1002E, reaction of Example 1053F (2.1 g, 4.57 mmol) and TFA (3.59 mL, 46.6 mmol) afforded Example 1053G (1.7 g, 98% yield) as brown solid. MS [M+H]⁺=373.2.

### Example 1053

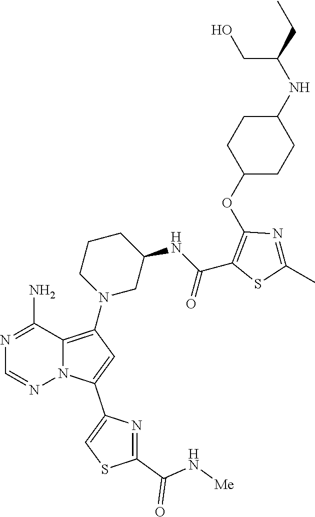 [SMILES: CC[C@H](CO)NC1CCC(Oc2nc(C)sc2C(=O)N[C@@H]2CCCN(c3cc(-c4csc(C(=O)NC)n4)n4ncnc(N)c34)C2)CC1]

Analogous to the preparation of Example 996, reaction of Example 1053B (88 mg, 0.26 mmol), Example 1053G (100 mg, 0.26 mmol), BOP (119 mg, 0.26 mmol) and DIPEA (0.14 mL, 0.80 mmol) afforded Example 1053 (27.7 mg, 15% yield), LC-MS Method E: RT=1.33 min, [M+H]⁺=683.2; LC-MS Method F: RT=0.99 min, [M+H]⁺=683.2; 1H NMR (400 MHz, DMSO-d₆) δ=8.82-8.71 (m, 1H), 8.54 (s, 1H), 8.09 (s, 1H), 7.96 (s, 1H), 7.29 (br s, 1H), 7.20 (s, 1H), 6.74 (s, 1H), 5.16 (br s, 1H), 4.19 (br s, 1H), 3.28-3.20 (m, 4H), 3.05 (br s, 1H), 2.85 (d, J=5.0 Hz, 4H), 2.61 (s, 4H), 2.43 (br s, 1H), 2.04-1.92 (m, 3H), 1.90-1.80 (m, 2H), 1.74-1.49 (m, 5H), 1.40-1.15 (m, 5H), 0.74 (t, J=7.5 Hz, 3H).

### Example 1054

### Preparation of 4-(4-amino-5-((R)-3-(4-(((1S,4S)-4-(tert-butylamino)cyclohexyl)oxy)-2-methylthiazole-5-carboxamido)piperidin-1-yl)pyrrolo[2,1-f][1,2,4]triazin-7-yl)-N-methylthiazole-2-carboxamide

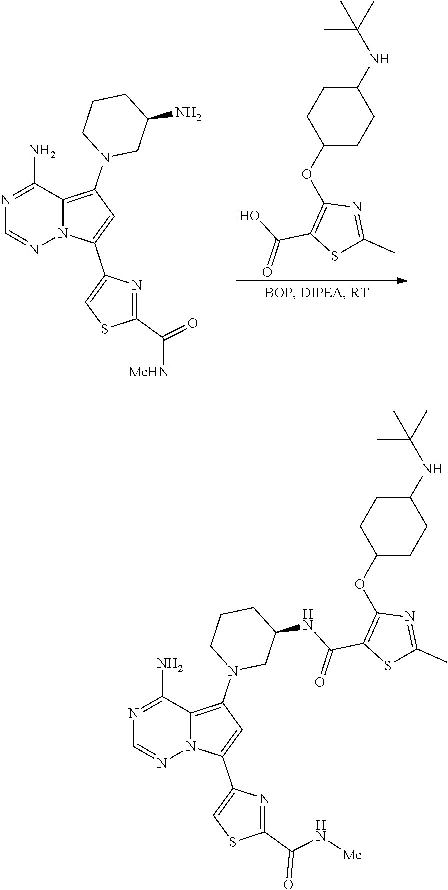 [SMILES: CNC(=O)c1nc(-c2cc(N3CCC[C@@H](N)C3)c3c(N)ncnn23)cs1 ; Cc1nc(OC2CCC(NC(C)(C)C)CC2)c(C(=O)O)s1 ; CNC(=O)c1nc(-c2cc(N3CCC[C@@H](NC(=O)c4sc(C)nc4OC4CCC(NC(C)(C)C)CC4)C3)c3c(N)ncnn23)cs1]

Analogous to the preparation of Example 996, reaction of Example 1026B (84 mg, 0.26 mmol), Example 1053G (100 mg, 0.26 mmol), BOP (119 mg, 0.26 mmol) and DIPEA (0.14 mL, 0.80 mmol) afforded Example 1054 (0.6 mg, 0.33% yield), LC-MS Method E: RT=1.38 min, [M+H]⁺=667.3; LC-MS Method F: RT=1.08 min, [M+H]⁺=667.3; ¹H NMR (400 MHz, DMSO-d₆) δ ppm 8.75 (br d, J=4.8 Hz, 1H), 8.54 (s, 1H), 8.09 (s, 1H), 7.96 (s, 1H), 7.29 (br s, 1H), 7.21 (s, 1H), 6.73 (s, 1H), 5.10 (br s, 1H), 4.21 (br s, 1H), 3.51 (s, 2H), 3.03 (br s, 1H), 2.85 (d, J=4.8 Hz, 5H), 2.61 (s, 3H), 2.07-1.77 (m, 7H), 1.62 (br s, 3H), 1.03 (br dd, J=5.8, 11.8 Hz, 1H).

### Example 1055

### Preparation of (R)—N-(1-(4-amino-7-(5-carbamoyl-6-fluoropyridin-2-yl)pyrrolo[2,1-f][1,2,4]triazin-5-yl)piperidin-3-yl)-2-methyl-4-((4-(tert-pentylamino)cyclohexyl)oxy)thiazole-5-carboxamide

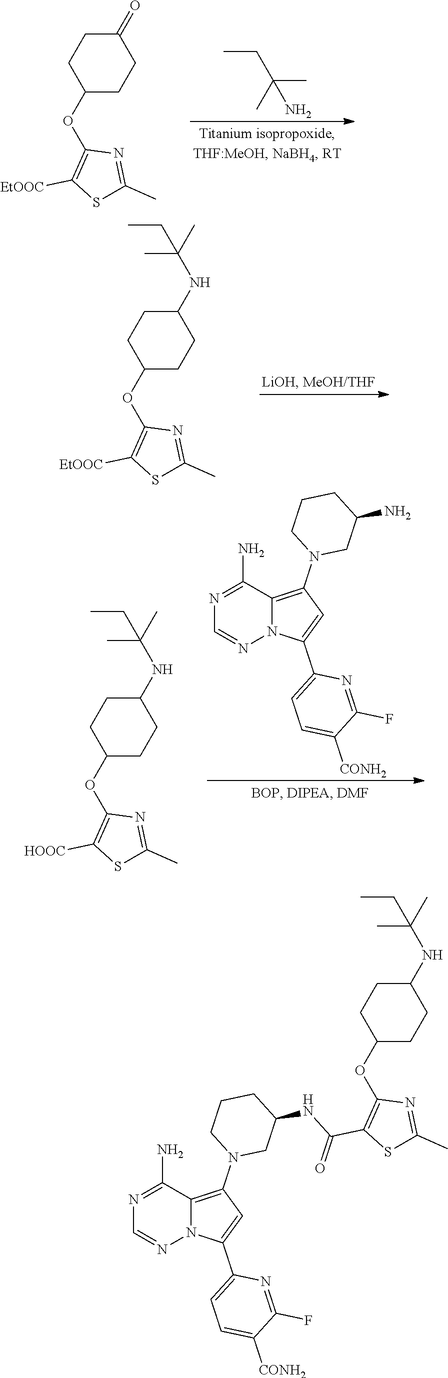

### Example 1055A. Preparation of ethyl 2-methyl-4-((4-(tert-pentylamino)cyclohexyl)oxy)thiazole-5-carboxylate

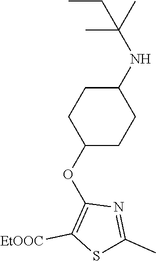 [SMILES: CCOC(=O)c1sc(C)nc1OC1CCC(NC(C)(C)CC)CC1]

Analogous to the preparation of Example 1000, reaction of Example 941B (4 g, 14.12 mmol), titanium(IV) isopropoxide (8.02 g, 28.2 mmol), 2-methylbutan-2-amine (7.38 g, 85 mmol) and sodium borohydride (1.06 g, 28.2 mmol) afforded Example 1055A (900 mg, 18% yield) as brown solid. MS: [M+H]⁺=355.3

### Example 1055B. Preparation of 2-methyl-4-((4-(tert-pentylamino)cyclohexyl)oxy)thiazole-5-carboxylic acid

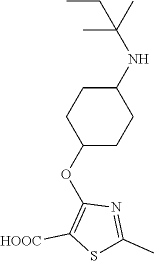 [SMILES: CCC(C)(C)NC1CCC(Oc2nc(C)sc2C(=O)O)CC1]

Analogous to the preparation of Example 996G, reaction of Example 1055A (900 mg. 2.54 mmol) and LiOH (152 mg, 6.35 mmol) afforded Example 1055B (700 mg, 84% yield) as brown solid. MS: [M+H]⁺=327.1.

### Example 1055

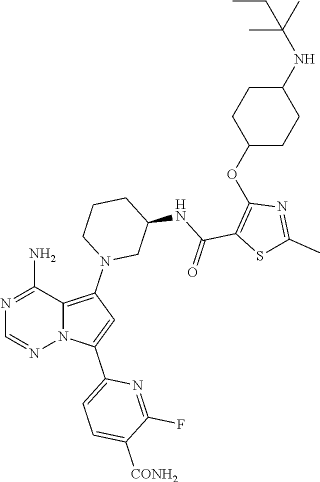 [SMILES: CCC(C)(C)NC1CCC(Oc2nc(C)sc2C(=O)N[C@@H]2CCCN(c3cc(-c4ccc(C(N)=O)c(F)n4)n4ncnc(N)c34)C2)CC1]

Analogous to the preparation of Example 996, reaction of Example 1040B (100 mg, 0.27 mmol), Example 1055B (88 mg, 0.27 mmol), BOP (119 mg, 0.27 mmol) and DIPEA (0.19 mL, 1.08 mmol) afforded Example 1055 (24.2 mg, 13% yield). LC-MS Method E: RT=1.42 min, [M+H]⁺=679.3; LC-MS Method F: RT=1.08 min, [M+H]⁺=679.3

### Example 1056

### Preparation of (R)—N-(1-(4-amino-7-(5-carbamoyl-6-fluoropyridin-2-yl)pyrrolo[2,1-f][1,2,4]triazin-5-yl)piperidin-3-yl)-4-((4-(tert-butylamino)cyclohexyl)oxy)-2-methylthiazole-5-carboxamide

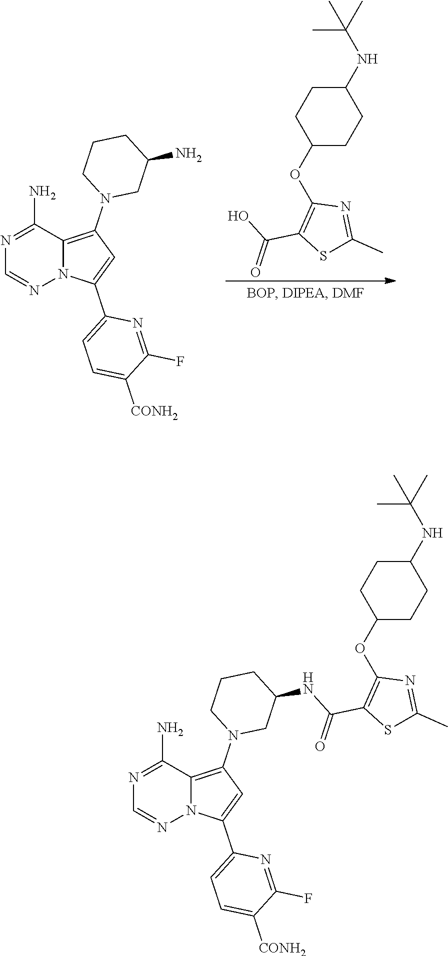 [SMILES: NC(=O)c1ccc(-c2cc(N3CCC[C@@H](N)C3)c3c(N)ncnn23)nc1F ; Cc1nc(OC2CCC(NC(C)(C)C)CC2)c(C(=O)O)s1 ; Cc1nc(OC2CCC(NC(C)(C)C)CC2)c(C(=O)N[C@@H]2CCCN(c3cc(-c4ccc(C(N)=O)c(F)n4)n4ncnc(N)c34)C2)s1]

Analogous to the preparation of Example 996, reaction of Example 1040B (100 mg, 0.27 mmol), Example 1026B (84 mg, 0.27 mmol), BOP (119 mg, 0.27 mmol) and DIPEA (0.19 mL, 1.08 mmol) afforded Example 1056 (25.7 mg, 14% yield). LC-MS Method E: RT=1.30 min, [M+H]⁺=665.3; LC-MS Method F: RT=1.03 min, [M+H]⁺=665.3; ₁H NMR (400 MHz, DMSO-d₆) δ=9.09-8.79 (m, 1H), 8.66 (dd, J=2.3, 8.0 Hz, 1H), 8.49 (br d, J=3.5 Hz, 1H), 8.43-8.16 (m, 1H), 8.10 (br s, 1H), 8.02 (s, 1H), 7.82 (br s, 1H), 7.29 (s, 1H), 7.13 (br s, 2H), 5.16-4.92 (m, 1H), 4.15 (br s, 1H), 3.45 (br d, J=7.3 Hz, 2H), 3.22-3.17 (m, 1H), 3.07 (br s, 1H), 2.84 (br s, 1H), 2.26 (s, 3H), 2.20-1.96 (m, 2H), 1.94-1.63 (m, 5H), 1.52 (br s, 1H), 1.30 (s, 9H).

### Example 1057

### N—((R)-1-(4-amino-7-(5-carbamoyl-6-methoxypyridin-2-yl)pyrrolo[2,1-f][1,2,4]triazin-5-yl)piperidin-3-yl)-4-(((1S,4S)-4 (dimethylamino)cyclohexyl)oxy)-2-methylthiazole5-carboxamide

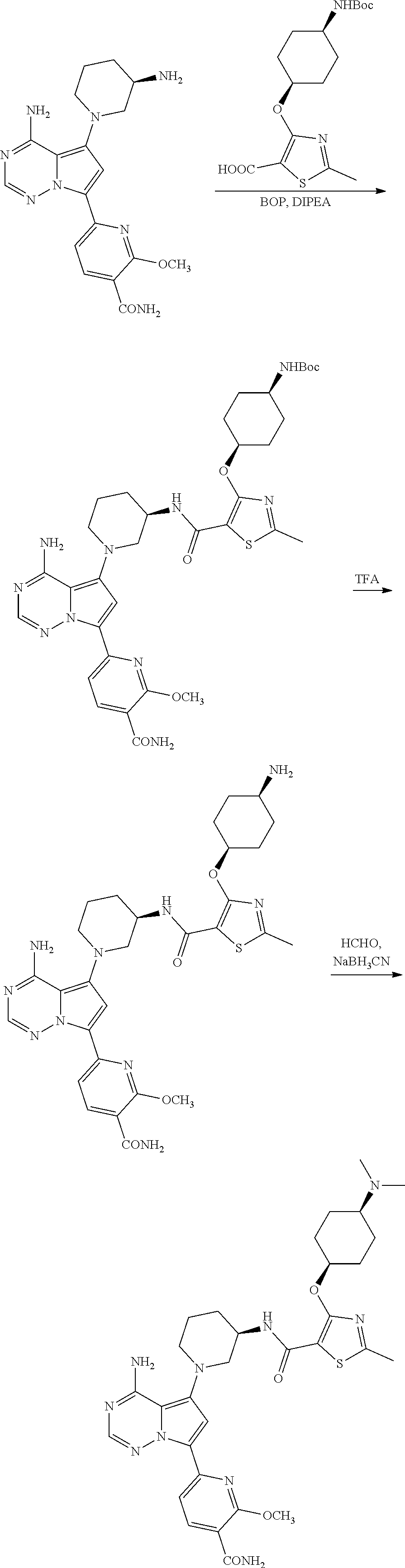 [SMILES: COc1nc(-c2cc(N3CCC[C@@H](N)C3)c3c(N)ncnn23)ccc1C(N)=O ; Cc1nc(O[C@H]2CC[C@@H](NC(=O)OC(C)(C)C)CC2)c(C(=O)O)s1 ; COc1nc(-c2cc(N3CCC[C@@H](NC(=O)c4sc(C)nc4O[C@H]4CC[C@@H](NC(=O)OC(C)(C)C)CC4)C3)c3c(N)ncnn23)ccc1C(N)=O ; COc1nc(-c2cc(N3CCC[C@@H](NC(=O)c4sc(C)nc4O[C@H]4CC[C@@H](N)CC4)C3)c3c(N)ncnn23)ccc1C(N)=O ; COc1nc(-c2cc(N3CCC[C@@H](NC(=O)c4sc(C)nc4O[C@H]4CC[C@@H](N(C)C)CC4)C3)c3c(N)ncnn23)ccc1C(N)=O]

### Example 1057A. Preparation of tert-butyl ((1S,4S)-4-((5-(((R)-1-(4-amino-7-(5-carbamoyl-6-methoxypyridin-2-yl)pyrrolo[2,1-f][1,2,4]triazin-5-yl)piperidin-3-yl)carbamoyl)-2-methylthiazol-4-yl)oxy)cyclohexyl)carbamate

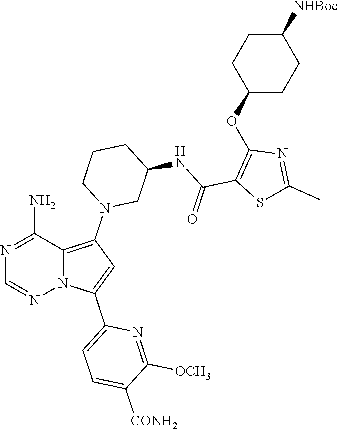 [SMILES: COc1nc(-c2cc(N3CCC[C@@H](NC(=O)c4sc(C)nc4O[C@H]4CC[C@@H](NC(=O)OC(C)(C)C)CC4)C3)c3c(N)ncnn23)ccc1C(N)=O]

Analogous to the preparation of Example 996, reaction of Example 1043D (250 mg, 0.65 mmol), Example 1002B (233 mg, 0.65 mmol). BOP (289 mg, 0.65 mmol) and DIPEA (0.34 mL, 1.96 mmol) afforded Example 1057A (300 mg, 64% yield) as yellow solid. MS [M+H]⁺=721.4.

### Example 1059B. Preparation of N—((R)-1-(4-amino-7-(5-carbamoyl-6-methoxypyridin-2-yl)pyrrolo[2,1-f][1,2,4]triazin-5-yl)piperidin-3-yl)-4-(((1S,4S)-4-aminocyclohexyl)oxy)-2-methylthiazole-5-carboxamide. TFA

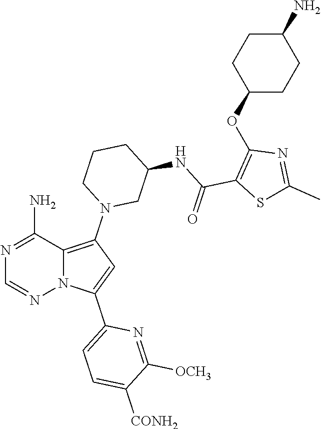 [SMILES: COc1nc(-c2cc(N3CCC[C@@H](NC(=O)c4sc(C)nc4O[C@H]4CC[C@@H](N)CC4)C3)c3c(N)ncnn23)ccc1C(N)=O]

Analogous to the preparation of Example 1002E, reaction of Example 1057A (300 mg, 0.42 mmol) and TFA (0.48 mL, 6.24 mmol) afforded Example 1057B (240 mg, 93% yield) as yellow solid. MS [M+H]⁺=621.4.

### Example 1057

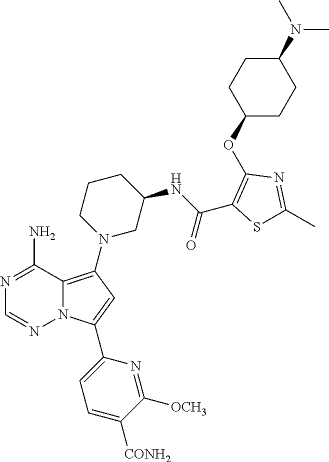 [SMILES: COc1nc(-c2cc(N3CCC[C@@H](NC(=O)c4sc(C)nc4O[C@H]4CC[C@@H](N(C)C)CC4)C3)c3c(N)ncnn23)ccc1C(N)=O]

Analogous to the preparation of Example 996B, reaction of Example 1057B (150 mg, 0.24 mmol), acetic acid (0.07 mL, 1.21 mmol), formaldehyde (0.03 mL, 1.21 mmol) and sodium cyanoborohydride (76 mg, 1.21 mmol) afforded Example 1057 (35 mg, 22% yield). LC-MS Method X: RT=1.57 min, [M+H]⁺=649.3; ¹H NMR (400 MHz, DMSO-d₆) δ ppm ¹H NMR (400 MHz, DMSO-d₆) δ ppm 8.40 (d, J=8.0 Hz, 1H), 8.29 (d, J=8.0 Hz, 1H), 8.25-8.10 (m, 1H), 7.99 (s, 1H), 7.68 (br s, 2H), 7.33 (s, 1H), 7.29-7.09 (m, 1H), 7.06-6.68 (m, 1H), 5.23-4.98 (m, 1H), 4.26-4.15 (m, 1H), 4.08 (s, 3H), 3.08-2.77 (m, 3H), 2.70-2.64 (m, 1H), 2.61 (s, 3H), 2.36-2.30 (m, 1H), 2.19-1.94 (m, 10H), 1.84 (br s, 2H), 1.73-1.42 (m, 6H).

### Example 1058

### Preparation of N—((R)-1-(4-amino-7-(6-methoxy-5-(methylcarbamoyl)pyridin-2-yl)pyrrolo[2,1-f][1,2,4]triazin-5-yl)piperidin-3-yl)-4-(((1S,4S)-4-(dimethylamino)cyclohexyl)oxy)-2-methylthiazole-5-carboxamide

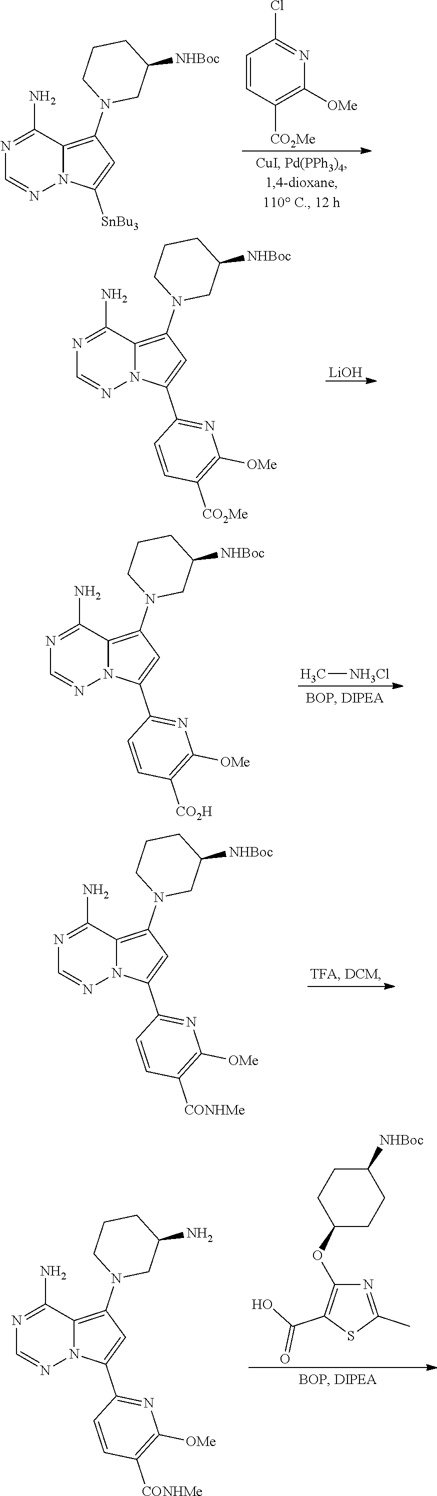

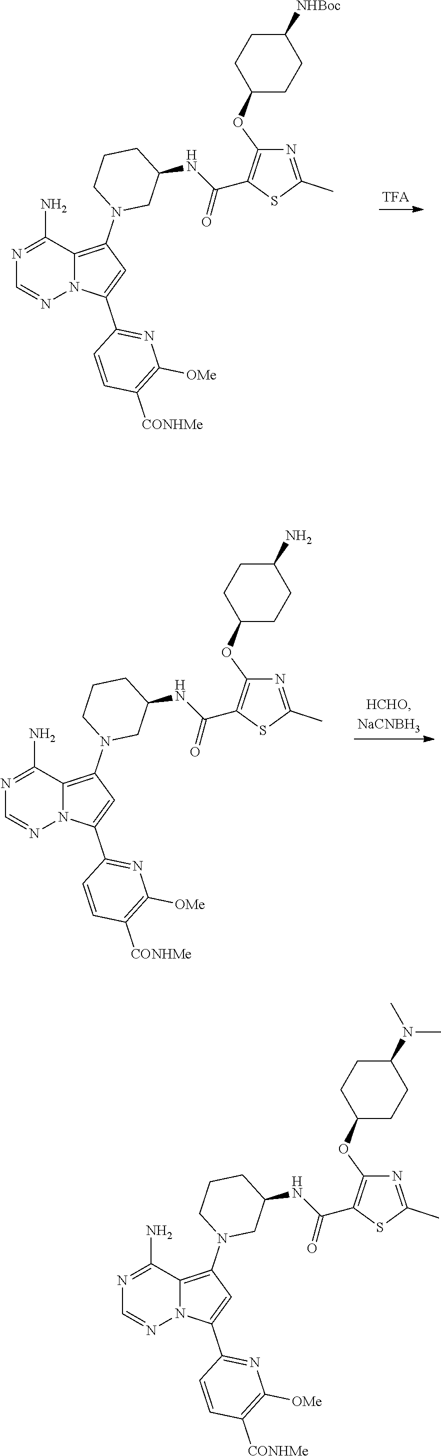

### Example 1058A. Preparation of methyl (R)-6-(4-amino-5-(3-((tert-butoxycarbonyl)amino)piperidin-1-yl)pyrrolo[2,1-f][1,2,4]triazin-7-yl)-2-methoxynicotinate

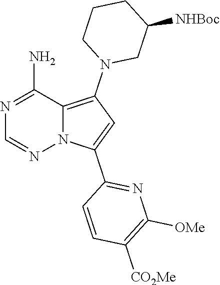 [SMILES: COC(=O)c1ccc(-c2cc(N3CCC[C@@H](NC(=O)OC(C)(C)C)C3)c3c(N)ncnn23)nc1OC]

Analogous to the preparation of Example 1026D, reaction of Example 1026C (1 g, 1.61 mmol), methyl 6-chloro-2-methoxynicotinate (0.324 g, 1.61 mmol), copper(I) iodide (0.03 g, 0.16 mmol) and Pd(PPh₃)₄ (0.18 g, 0.16 mmol) afforded Example 1058A (600 mg, 75% yield) as brown solid. MS [M+H]⁺=498.3.

### Example 1058B. Preparation of (R)-6-(4-amino-5-(3-((tert-butoxycarbonyl)amino)piperidin-1-yl)pyrrolo[2,1-f][1,2,4]triazin-7-yl)-2-methoxynicotinic acid

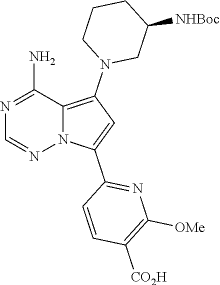 [SMILES: COc1nc(-c2cc(N3CCC[C@@H](NC(=O)OC(C)(C)C)C3)c3c(N)ncnn23)ccc1C(=O)O]

Analogous to the preparation of Example 9% C, reaction of Example 1058A (600 mg, 1.21 mmol) and LiOH (72.2 mg, 3.01 mmol) afforded Example 1059B (500 mg, 80% yield). MS [M+H]⁺=498.3.

### Example 1058C. Preparation of tert-butyl (R)-(1-(4-amino-7-(6-methoxy-5-(methylcarbamoyl)pyridin-2-yl)pyrrolo[2,1-f][1,2,4]triazin-5-yl)piperidin-3-yl)carbamate

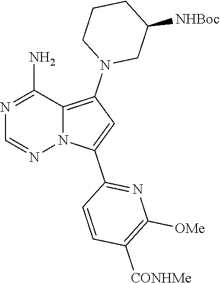

Analogous to the preparation of Example 996, reaction of Example 1058B (880 mg, 1.82 mmol), methylamine hydrochloride (369 mg, 5.46 mmol), BOP (805 mg, 1.82 mmol) and DIPEA (0.95 mL, 5.46 mmol) afforded Example 1058C (590 mg, 65% yield) as a brown solid. MS: [M+H]⁺=497.3.

### Example 1058D. Preparation of (R)-6-(4-amino-5-(3-aminopiperidin-1-yl)pyrrolo[2,1-f][1,2,4]triazin-7-yl)-2-methoxy-N-methylnicotinamide

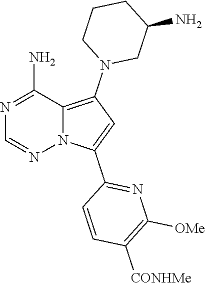

Analogous to the preparation of Example 1002E, reaction of Example 1058C (590 mg, 1.18 mmol) and TFA (0.91 mL, 11.88 mmol) afforded Example 1058D (450 mg, 90% yield) as yellow solid. MS: [M+H]⁺=397.2.

### Example 1058E. Preparation of tert-butyl ((1S,4S)-4-((5-(((R)-1-(4-amino-7-(6-methoxy-5-(methylcarbamoyl)pyridin-2-yl)pyrrolo[2,1-f][1,2,4]triazin-5-yl)piperidin-3-yl)carbamoyl)-2-methylthiazol-4-yl)oxy)cyclohexyl)carbamate

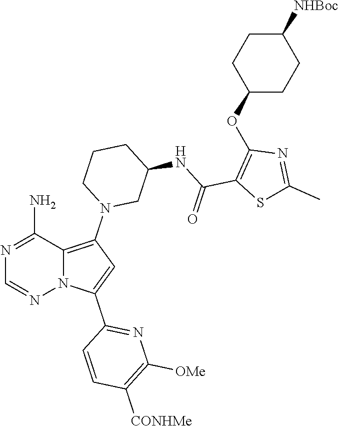

Analogous to the preparation of Example 996, reaction of Example 1002B (270 mg, 0.75 mmol), Example 1058D (300 mg, 0.75 mmol), BOP (335 mg, 0.75 mmol) and DIPEA (0.39 mL, 2.27 mmol) afforded Example 1058E (324 mg, 58% yield) as yellow solid. MS: [M+H]⁺=735.4.

### Example 1058F. Preparation of tert-butyl ((1S,4S)-4-((5-(((R)-1-(4-amino-7-(6-methoxy-5-(methylcarbamoyl)pyridin-2-yl)pyrrolo[2,1-f][1,2,4]triazin-5-yl)piperidin-3-yl)carbamoyl)-2-methylthiazol-4-yl)oxy)cyclohexyl)carbamate. TFA

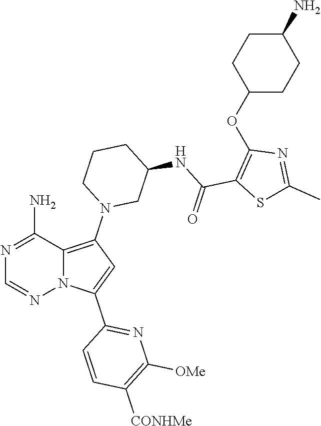

Analogous to the preparation of Example 1002E, reaction of Example 1058E (324 mg, 0.44 mmol) and TFA (0.34 mL, 4.41 mmol) afforded Example 1058F (280 mg, 100% yield) as yellow solid. MS: [M+H]⁺=635.3

### Example 1058

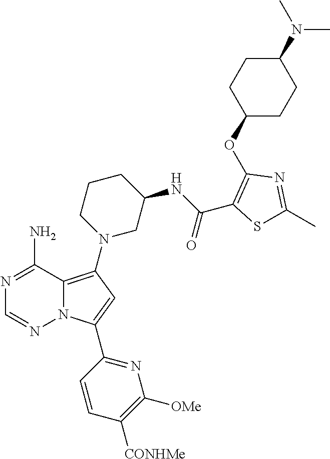

Analogous to the preparation of Example 996, reaction of Example 1058F (45 mg, 0.07 mmol), sodium cyanoborohydride (22.28 mg, 0.35 mmol), acetic acid (0.04 mL, 0.71 mmol) and formaldehyde (0.02 mL, 0.71 mmol) afforded Example 1058 (2.6 mg, 6% yield). LC-MS Method E: RT=1.38 min, [M+H]⁺=663.0; LC-MS Method F: RT=1.04 min. [M+H]⁺=663.0; ¹H NMR (400 MHz, DMSO-d₆) δ ppm 8.39 (d, J=8.0 Hz, 1H), 8.28-8.24 (m, 1H), 8.21 (br d, J=5.3 Hz, 2H), 7.98 (s, 1H), 7.32 (s, 1H), 7.26-6.83 (m, 1H), 5.13 (br s, 1H), 4.20 (br s, 1H), 4.08 (s, 3H), 3.46-3.34 (m, 4H) 3.02 (br s, 1H), 2.83 (d, J=4.8 Hz, 5H), 2.60 (s, 3H), 2.28-1.94 (m, 9H), 1.84 (td, J=1.3, 2.9 Hz, 2H), 1.75-1.40 (m, 5H).

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

1. A compound according to Formula (I): 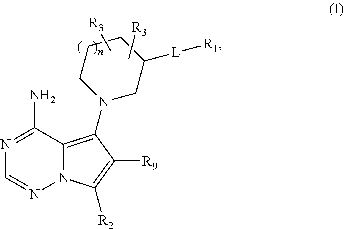
or a pharmaceutically acceptable salt thereof, wherein:
L is selected from the group consisting of —C(═O)NR₈—, —C(═O)O—, —NR₈C(═O)—, —NR₈C(═O)NR₈—, —NR₈C(═O)O—, and —NR₈S(═O)ₚ—;
R₁ is selected from the group consisting of C₁₋₅ alkyl, —(CR_{d}R_{d})ᵣ—C₃₋₁₀ carbocyclyl substituted with 1-5 R₄, and 4- to 10-membered heterocyclyl comprising carbon atoms and 1-5 heteroatoms selected from N, NR₄ₐ, O, and S(═O)ₚ, and substituted with 1-5 R₄;
R₂ is selected from the group consisting of CN, C(═O)NHR₉, C₃₋₁₀ carbocyclyl substituted with 1-5 R₆, 3- to 10-membered heterocyclyl comprising carbon atoms and 1-5 heteroatoms selected from N, NR₆ₐ, O, and S(═O)ₚ, and substituted with 1-5 R₆, and C₂₋₃ alkynyl substituted with 0-1 C₃₋₁₀ carbocyclyl substituted with 1-5 R₆ or 3- to 10-membered heterocyclyl comprising carbon atoms and 1-5 heteroatoms selected from N, NR₆ₐ, O, and S(═O)ₚ, and substituted with 1-5 R₆;
R₃ is selected from the group consisting of H, halo, —OR_{b}, —NRₐRₐ and C₁₋₃ alkyl substituted with 0-4 halo, OR_{b}, or NRₐRₐ substituents; alternatively, two adjacent R₃ groups are taken together with the carbon atoms to which they are attached to form a C₃₋₆ cycloalkyl;
R₄, at each occurrence, is independently selected from the group consisting of H, F, Cl, Br, ═O, CN, NO₂, —OR_{b}, —(CR_{d}R_{d})NRₐRₐ, —O(CR_{d}R_{d})₁₋₅OR_{b}, —O(CR_{d}R_{d})ᵣC(═O)NRₐRₐ, —O(CR_{d}R_{d})₁₋₅NRₐC(═O)R_{b}, —O(CR_{d}R_{d})₁₋₅NRₐC(═O)OR_{b}, —O(CR_{d}R_{d})₁₋₅NRₐRₐ, —C(═O)NRₐRₐ, —C(═O)R_{b}, —NRₐC(═O)OR_{b}, —NRₐC(═O)(CR_{d}R_{d})ᵣNRₐRₐ, S(═O)R_{c}, S(═O)NRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₁₂ carbocyclyl substituted with 1-5 R₅, —(CR_{d}R_{d})ᵣ—O—(CR_{d}R_{d})ᵣ—C₃₋₁₂ carbocyclyl substituted with 1-5 R₅, —(CR_{d}R_{d})ᵣ-3- to 18-membered heterocyclyl comprising carbon atoms and 1-4 heteroatoms selected from N, NR₅ₐ, O, and S(═O)ₚ, and substituted with 1-5 R₅; and —(CR_{d}R_{d})ᵣ—O—(CR_{d}R_{d})ᵣ-3- to 18-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣ—C₃₋₁₀ carbocyclyl substituted with 1-5 R₅, and —(CR_{d}R_{d})ᵣ-3- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CR_{d}R_{d})ᵣ—NR₁₀R₁₀, C₁₋₆ alkyl substituted with 1-5 Rₑ, C₂₋₆ alkenyl substituted with 1-5 Rₑ, C₂₋₄ alkynyl substituted with 1-5 Rₑ, C₃₋₁₀ carbocyclyl with 1-5 Rₑ, and 3- to 10-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₆ alkyl substituted with 1-5 Rₑ, C(═O)R_{b}, C(═O)OR_{b}, C(═O)NRₐRₐ, S(═O)ₚR_{c}, S(═O)ₚNRₐRₐ, C₃₋₁₀ carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₆ alkyl substituted with 1-5 Rₑ;
R₆, at each occurrence, is independently selected from the group consisting of H, F, Cl, Br, CN, ═O, —(CR_{d}R_{d})NRₐRₐ, NO₂, —OR_{b}, —C(═O)NRₐRₐ, —C(═O)NRₐ(CR_{d}R_{d})ᵣOR_{b}, —C(═O)R_{b}, —C(═O)(CR_{d}R_{d})ᵣOR_{b}, —NRₐC(═O)OR_{b}, —NRₐC(═O)(CR_{d}R_{d})ᵣNRₐRₐ, —S(═O)_{P}R_{c}, —NRₐS(═O)_{P}R_{c}, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣ—C₃₋₁₀ carbocyclyl substituted with 1-5 R₇, and —(CR_{d}R_{d})ᵣ-3- to 10-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —(CR_{d}R_{d})ᵣC(═O)NRₐRₐ, —(CR_{d}R_{d})ᵣC(═O)R_{b}, —(CR_{d}R_{d})C(═O)(CR_{d}R_{d})OR_{b}, —(CR_{d}R_{d})ᵣC(═O)(CR_{d}R_{d})ᵣNRₐC(═O)R_{b}, —(CR_{d}R_{d})ᵣS(═O)ₚR_{c}, —(CR_{d}R_{d})ᵣS(═O)NRₐRₐ, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₁₀ carbocyclyl substituted with 1-5 R₇, and —(CR_{d}R_{d})ᵣ-3- to 10-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₆ alkyl substituted with 1-5 Rₑ, C₂₋₆ alkenyl substituted with 1-5 Rₑ, and C₂₋₆ alkynyl substituted with 1-5 Rₑ;
R₇ₐ, at each occurrence, is independently selected from the group consisting of H and C₁₋₆ alkyl substituted with 1-5 Rₑ;
R₈, at each occurrence, is independently selected from the group consisting of H and C₁₋₃ alkyl substituted with 0-4 halo, OR_{b}, or NRₐRₐ substituents;
R₉ is selected from the group consisting of H and C₁₋₃ alkyl;
R₁₀, at each occurrence, is independently selected from the group consisting of H, C₁₋₇ alkyl substituted with 1-5 Rₑ, —(CR_{d}R_{d})ᵣ—C₃₋₁₀ carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
Rₐ, at each occurrence, is independently selected from the group consisting of H, —C(═O)OR_{b}, C₁₋₆ alkyl substituted with 1-5 Rₑ, C₂₋₆ alkenyl substituted with 1-5 Rₑ, C₂₋₄ alkynyl substituted with 1-5 Rₑ, —(CR_{d}R_{d})ᵣ—C₃₋₁₀ carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or Rₐ and Rₐ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
R_{b}, at each occurrence, is independently selected from the group consisting of H, C₁₋₆ alkyl substituted with 1-5 Rₑ, C₂₋₆ alkenyl substituted with 1-5 Rₑ, C₂₋₆ alkynyl substituted with 1-5 Rₑ, —(CR_{d}R_{d})ᵣC₃₋₁₀carbocyclyl substituted with 1-5 Rₑ, and —(CR_{d}R_{d})ᵣ-3- to 10-membered heterocyclyl comprising carbon atoms and 1-4 heteroatoms selected from the group consisting of N, NR_{f}, O, and S(═O)ₚ, and substituted with 1-5 Rₑ;
R_{c}, at each occurrence, is independently selected from the group consisting of F, Cl, C₁₋₆ alkyl substituted with 1-5 Rₑ, C₂₋₆alkenyl substituted with 1-5 Rₑ, C₂₋₆alkynyl substituted with 1-5 Rₑ, C₃₋₁₀ carbocyclyl substituted with 1-5 Rₑ, and 3- to 10-membered heterocyclyl comprising carbon atoms and 1-4 heteroatoms selected from the group consisting of N, NR_{f}, O, and S(═O)ₚ, and substituted with 1-5 Rₑ;
R_{d}, at each occurrence, is independently selected from the group consisting of H, C₁₋₆ alkyl substituted with 1-5 Rₑ, and C₃₋₆ cycloalkyl substituted with 1-5 Rₑ;
Rₑ is independently selected from the group consisting of H, F, Cl, Br, CN, ═O, C₁₋₆alkyl substituted with 1-5 R_{g}, C₂₋₆alkenyl substituted with 1-5 R_{g}, C₂₋₆alkynyl substituted with 1-5 R_{g}, —(CH₂)ᵣ—C₃₋₁₀ carbocyclyl substituted with 1-5 R_{g}, —(CH₂)ᵣ-3- to 10-membered heterocyclyl comprising carbon atoms and 1-4 heteroatoms selected from the group consisting of N, NR_{f}, O, and S(═O)ₚ, and substituted with 1-5 R_{g}, —(CH₂)ᵣOR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₆alkyl substituted with 1-5 R_{g}, C₃₋₁₀ carbocyclyl substituted with 1-5 R_{g}, a 3- to 10-membered heterocyclyl comprising carbon atoms and 1-4 heteroatoms selected from the group consisting of N, NC₁₋₄ alkyl, O, and S(═O)ₚ, and substituted with 1-3 R_{g}; or R_{f} and R_{f} together with the nitrogen atom to which they are both attached form a 3- to 10-membered heterocyclyl comprising carbon atoms and 1-4 heteroatoms selected from the group consisting of N, NC₁₋₄ alkyl, O, and S(═O)ₚ, and substituted with 1-5 R_{g};
R_{g}, at each occurrence, is independently selected from the group consisting of H, F, Cl, Br, CN, —OH, —O(C₁₋₅ alkyl), NH₂, NH(C₁₋₅ alkyl), NH(C₁₋₅ alkyl)₂, C₁₋₅ alkyl, C₃₋₁₀ carbocyclyl, and a 3- to 10-membered heterocyclyl comprising carbon atoms and 1-4 heteroatoms selected from the group consisting of N, NC₁₋₄ alkyl, O, and S(═O)ₚ;
n is an integer of zero, 1, or 2;
p is an integer of zero, 1, or 2; and
r is an integer of zero, 1, 2, 3, 4, or 5.

2. The compound of claim 1, having Formula (II): 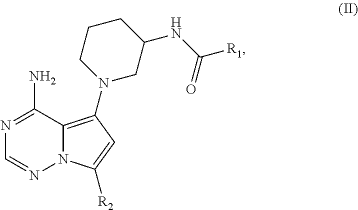
or a pharmaceutically acceptable salt thereof, wherein:
R₁ is selected from the group consisting of 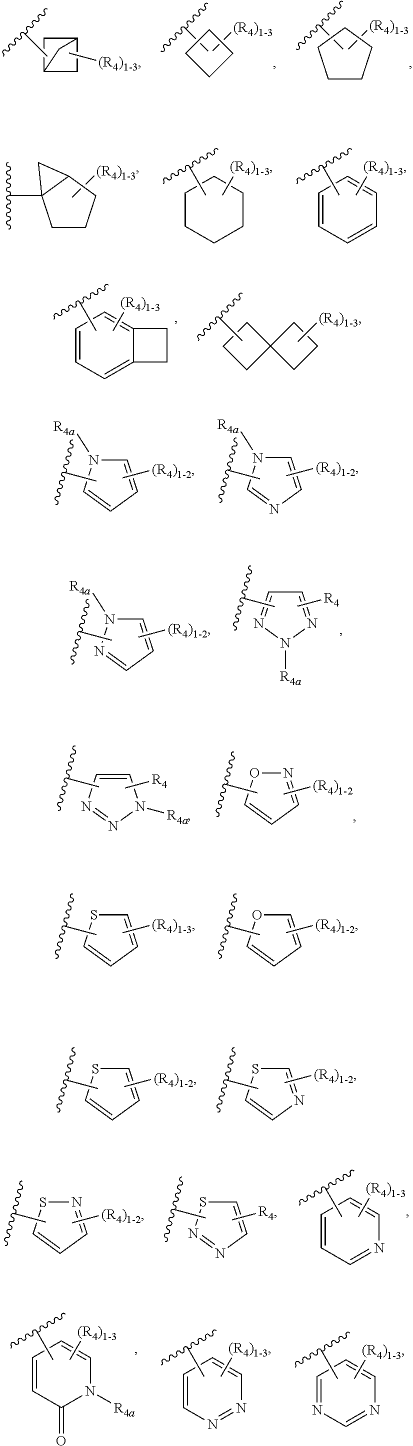 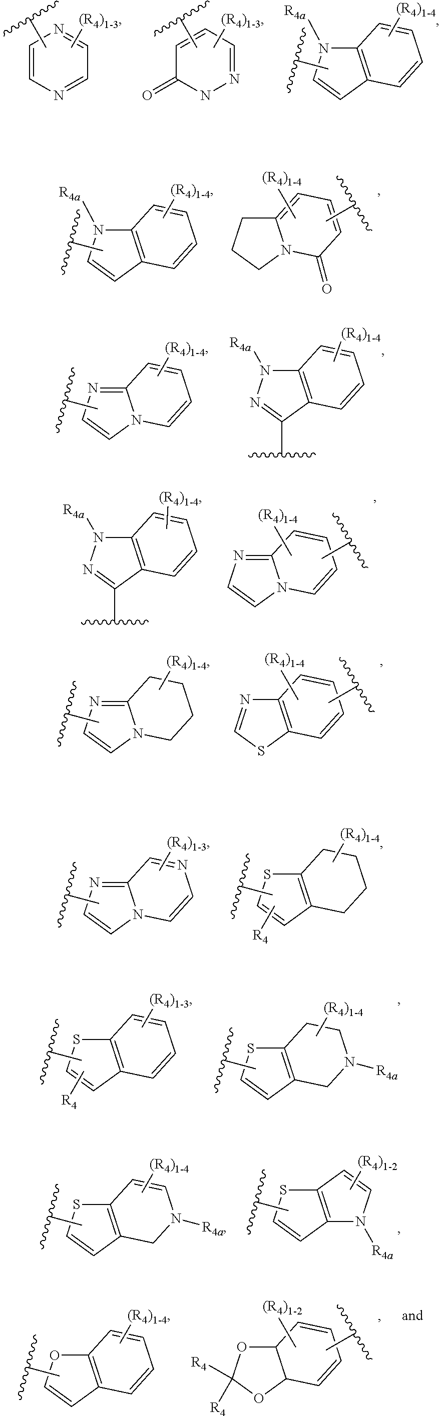 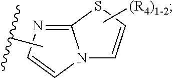
R₂ is selected from the group consisting of 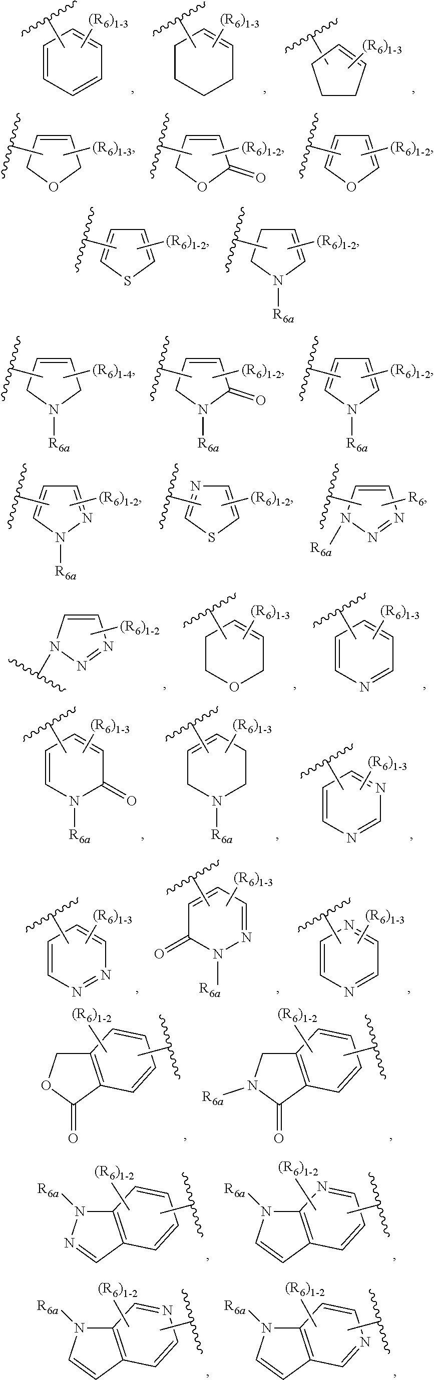 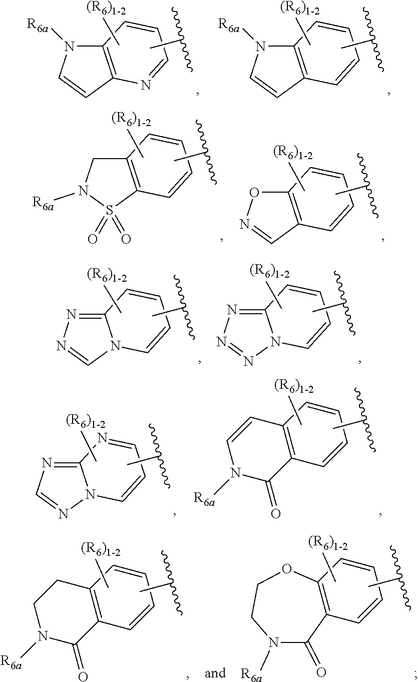
R₄, at each occurrence, is independently selected from the group consisting of H, F, Cl, Br, ═O, CN, —(CH₂)ᵣNRₐRₐ, —OR_{b}, —O(CH₂)₁₋₄OR_{b}, —O(CH₂)ᵣC(═O)NRₐRₐ, —O(CH₂)₁₋₄NRₐC(═O)R_{b}, —O(CH₂)₁₋₄NRₐC(═O)OR_{b}, —O(CH₂)₁₋₄NRₐRₐ, —C(═O)NRₐRₐ, —C(═O)R_{b}, —NRₐC(═O)OR_{b}, —NRₐC(═O)(CH₂)ᵣNRₐRₐ, C₁₋₅ alkyl substituted with 1-5 Rₑ, C₂₋₅ alkenyl substituted with 1-5 Rₑ, C₂₋₅ alkynyl substituted with 1-5 Rₑ; —(CH₂)ᵣ—C₃₋₁₀ carbocyclyl substituted with 1-5 R₅, —(CH₂)ᵣ—O—(CH₂)ᵣ—C₃₋₁₀ carbocyclyl substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, C₁₋₅ alkyl substituted with 1-5 Rₑ, C₂₋₅ alkenyl substituted with 1-5 Rₑ, —(CH₂)ᵣ—C₃₋₆ carbocyclyl substituted with 1-5 R₅, and —(CH₂)ᵣ-5- to 9-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, —OR_{b}, ═O, —(CH₂)ᵣ—NR₁₀R₁₀, C₁₋₅ alkyl substituted with 1-5 Rₑ, C₂₋₅ alkenyl substituted with 1-5 Rₑ, C₂₋₅ alkynyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₅ alkyl substituted with 1-5 Rₑ, C(═O)R_{b}, C(═O)OR_{b}, C(═O)NRₐRₐ, S(═O)ₚR_{c}, S(═O)NRₐRₐ, C₃₋₆carbocyclyl substituted with 1-5 Rₑ, and 5- to 9-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₅ alkyl substituted with 1-5 Rₑ;
R₆, at each occurrence, is independently selected from the group consisting of H, F, Cl, Br, CN, ═O, —(CH₂)ᵣNRₐRₐ, —OR_{b}, —C(═O)NRₐRₐ, —C(═O)NRₐ(CH₂)OR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)_{P}R_{c}, —NRₐS(═O)_{P}R_{c}, C₁₋₅ alkyl substituted with 1-5 Rₑ, C₂₋₅ alkenyl substituted with 1-5 Rₑ, —(CH₂)ᵣ—C₃₋₆carbocyclyl substituted with 1-5 R₇, and —(CH₂)ᵣ-5- to 9-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —(CH₂)C(═O)NRₐRₐ, —(CH₂)ᵣC(═O)R_{b}, —(CH₂)ᵣC(═O)(CHR_{d})ᵣOR_{b}, —(CH₂)ᵣS(═O)ₚR_{c}, —(CH₂)ᵣS(═O)ₚNRₐRₐ, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)ᵣ—C₃₋₆ carbocyclyl substituted with 1-5 R₇, and —(CH₂)ᵣ-4- to 9-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₅ alkyl substituted with 1-5 Rₑ, C₂₋₅ alkenyl substituted with 1-5 Rₑ, and C₂₋₅ alkynyl substituted with 1-5 Rₑ;
R₇ₐ, at each occurrence, is independently selected from the group consisting of H and C₁₋₅ alkyl substituted with 1-5 Rₑ;
R₁₀, at each occurrence, is independently selected from the group consisting of H, C₁₋₅ alkyl substituted with 1-5 Rₑ, —(CH₂)ᵣ—C₃₋₁₀ carbocyclyl substituted with 1-5 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-5 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-5 Rₑ;
Rₐ is independently selected from the group consisting of H, C(═O)OR_{b}, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)ᵣ—C₃₋₁₀ carbocyclyl substituted with 1-5 Rₑ, and —(CH₂)ᵣ-3- to 9-membered heterocyclyl comprising carbon atoms and 1-4 heteroatoms selected from the group consisting of N, NR_{f}, O, and S(═O)ₚ, and substituted with 1-5 Rₑ; or Rₐ and Rₐ together with the nitrogen atom to which they are both attached form a 4- to 9-membered heterocyclyl comprising carbon atoms and 1-4 heteroatoms selected from the group consisting of N, NR_{f}, O, and S(═O)ₚ, and substituted with 1-5 Rₑ;
R_{b}, at each occurrence, is independently selected from the group consisting of H, C₁₋₅ alkyl substituted with 1-5 Rₑ, C₂₋₅ alkenyl substituted with 1-5 Rₑ, C₂₋₅ alkynyl substituted with 1-5 Rₑ, —(CH₂)—C₃₋₁₀ carbocyclyl substituted with 1-5 Rₑ, and —(CH₂)ᵣ-4- to 9-membered heterocyclyl comprising carbon atoms and 1-4 heteroatoms selected from the group consisting of N, NR_{f}, O, and S(═O)ₚ, and substituted with 1-5 Rₑ;
R_{c}, at each occurrence, is independently selected from the group consisting of C₁₋₅ alkyl substituted with 1-5 Rₑ, C₂₋₅ alkenyl substituted with 1-5 Rₑ, C₂₋₅ alkynyl substituted with 1-5 Rₑ, C₃₋₆carbocyclyl substituted with 1-5 Rₑ, and 4- to 9-membered heterocyclyl comprising carbon atoms and 1-4 heteroatoms selected from the group consisting of N, NR_{f}, O, and S(═O)ₚ, and substituted with 1-5 Rₑ;
R_{d}, at each occurrence, is independently selected from the group consisting of H, C₁₋₅ alkyl substituted with 1-4 Rₑ, and C₃₋₆ cycloalkyl substituted with 1-4 Rₑ;
Rₑ, at each occurrence, is independently selected from the group consisting of H, F, Cl, Br, CN, ═O, C₁₋₅ alkyl substituted with 1-5 R_{g}, C₂₋₅ alkenyl substituted with 14 R_{g}, C₂₋₅ alkynyl substituted with 1-5 R_{g}, —(CH₂)ᵣC₃₄ cycloalkyl substituted with 1-5 R_{g}, —(CH₂)ᵣ-4- to 9-membered heterocyclyl comprising carbon atoms and 1-4 heteroatoms selected from the group consisting of N, NR_{f}, O, and S(═O)ₚ, and substituted with 1-5 R_{g}, —(CH₂)ᵣOR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a 4- to 9-membered heterocyclyl;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4 or 5.

3. The compound of claim 2, or a pharmaceutically acceptable salt thereof, wherein:
R₁ is selected from the group consisting of 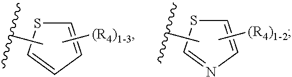
R₂ is selected from the group consisting of 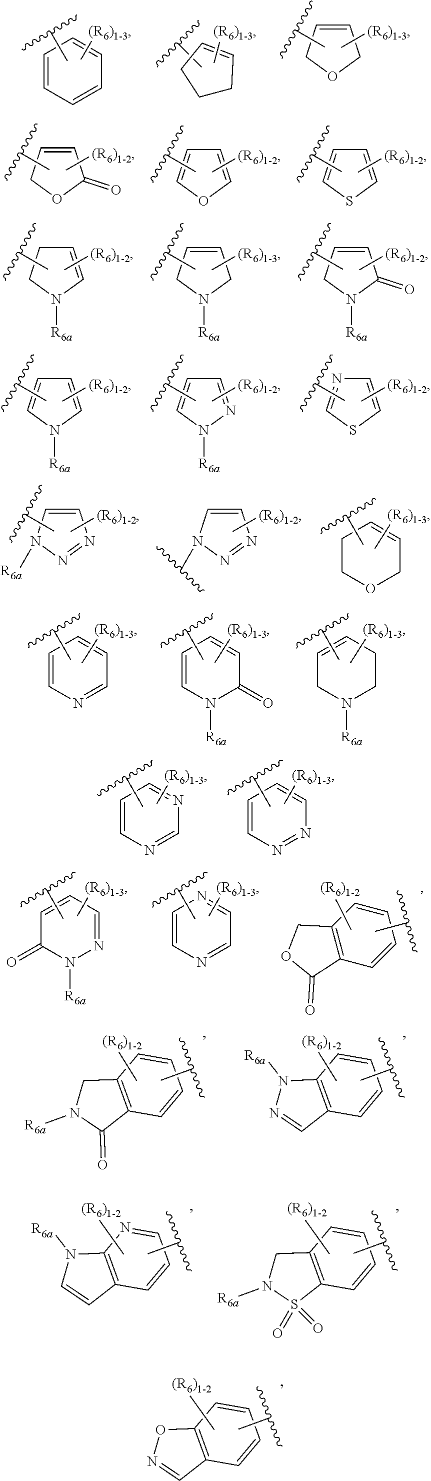 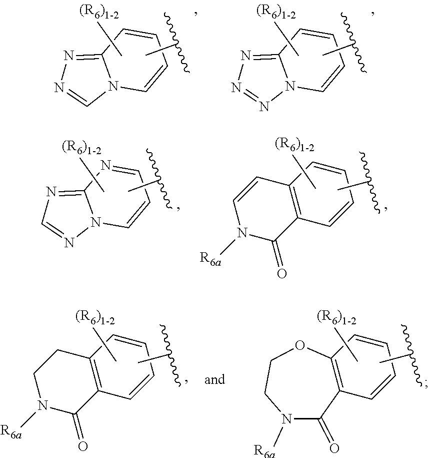
R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₁₀ carbocyclyl substituted with 1-5 R₅, —(CH₂)ᵣ—O—(CH₂)ᵣ—C₃₋₁₀ carbocyclyl substituted with 1-5 R₅, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CH₂)ᵣNR₁₀R₁₀, C₁₋₅ alkyl substituted with 1-4 Rₑ, C₃₋₆ carbocyclyl with 1-4 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H and C₁₋₅ alkyl substituted with 1-4 Rₑ, C(═O)R_{b}, C(═O)OR_{b}, C₃₋₆carbocyclyl substituted with 1-4 Rₑ, and 5- to 9-membered heterocyclyl comprising carbon atoms and 1-4 heteroatoms selected from the group consisting of N, NR_{5b}, O, and S(═O)ₚ, and substituted with 1-4 Rₑ;
R_{5b}, at each occurrence, is independently selected from the group consisting of H and C₁₋₄ alkyl substituted with 1-4 Rₑ;
R₆, at each occurrence, is independently selected from the group consisting of H, F, Cl, Br, CN, ═O, —(CH₂)ᵣNRₐRₐ, NO₂, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂Rₑ, —NRₐS(═O)₂Rₑ, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₄ alkynyl substituted with 1-5 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ, at each occurrence, is independently selected from the group consisting of H and C14 alkyl substituted with 1-5 Rₑ;
R₁₀, at each occurrence, is independently selected from the group consisting of H, C₁₋₅ alkyl substituted with 1-4 Rₑ, —(CH₂)ᵣ—C₃₋₁₀carbocyclyl substituted with 1-4 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-4 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-4 Rₑ;
Rₐ, at each occurrence, is independently selected from the group consisting of H, C₁₋₅ alkyl substituted with 1-4 Rₑ, C₃₋₁₀ carbocyclyl substituted with 1-4 Rₑ, and —(CH₂)ᵣ-4- to 9-membered heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a 4- to 9-membered heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 1-4 Rₑ, C₂₋₅ alkynyl substituted with 1-4 Rₑ, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c}, at each occurrence, is independently selected from the group consisting of C₁₋₅ alkyl substituted with 1-4 Rₑ, C₃₋₆carbocyclyl substituted with 1-4 Rₑ, and 4- to 8-membered heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H, C₁₋₄ alkyl, and C₃₋₆ cycloalkyl;
Rₑ, at each occurrence, is independently selected from the group consisting of H, F, Cl, Br, CN, ═O, C₁₋₅ alkyl substituted with 1-4 R_{g}, C₂₋₅ alkenyl substituted with 14 R_{g}, C₂₋₅ alkynyl substituted with 14 R_{g}, —(CH₂)ᵣC₃₋₆ cycloalkyl, —(CH₂)ᵣ-4- to 8-membered heterocyclyl comprising carbon atoms and 1-4 heteroatoms selected from the group consisting of N, NR_{f}, O, and S(═O)ₚ, and substituted with 1-4 R_{g}, —OR_{f}, —(CH₂)ᵣS(═O)₂C₁₋₅ alkyl, —(CH₂)ᵣNR_{f}R_{f}, —(CH₂)C(═O)R_{f}, and —(CH₂)C(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a 4- to 8-membered heterocyclic ring;
R_{g}, at each occurrence, is independently selected from the group consisting of H, F, Cl, Br, OH, CN, NH₂, C₁₋₄ alkyl, and C₃₋₆ cycloalkyl;
p is an integer of zero, 1, or 2; and
r is an integer of zero, 1, 2, 3, 4, or 5.

4. The compound of claim 3, or a pharmaceutically acceptable salt thereof, wherein:
R₁ is selected from the group consisting of 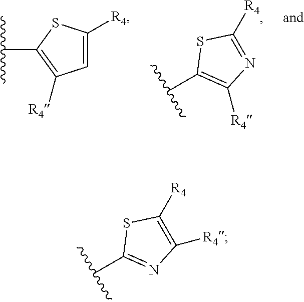
R₂ is selected from the group consisting, 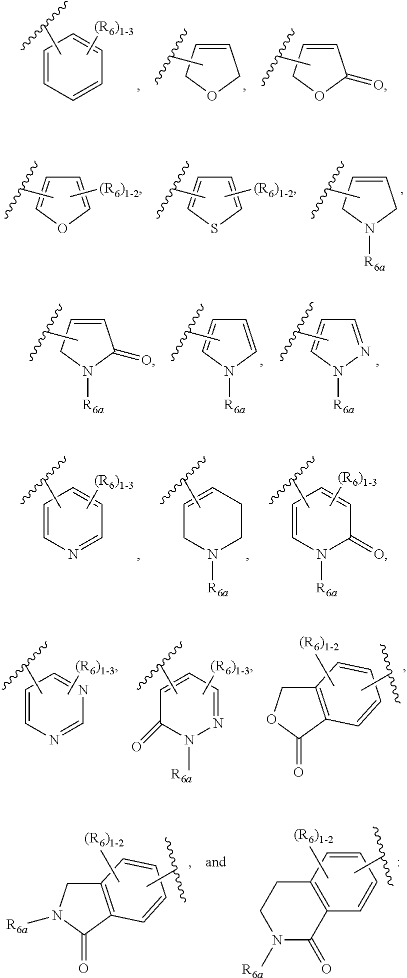
R₄′ is selected from the group consisting of H, F, Cl, CN, and C₁₋₄ alkyl substituted with 1-3 Rₑ;
R₄″ is selected from the group consisting of H, F, —O(CH₂)₁₋₃OR_{b}, —O(CH₂)₁₋₃C(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, C₃₋₁₀ carbocyclyl substituted with 1-4 R₅, —(CH₂)₀₋₄—O—(CH₂)₀₋₄ C₃₋₁₀ carbocyclyl substituted with 1-4 R₅, —(CH₂)₀₋₄-4- to 10-membered heterocyclyl comprising carbon atoms and 1-4 heteroatoms selected from N, NR₅ₐ, O, and S(═O)ₚ, and substituted with 1-4 R₅, and —(CH₂)₄—O—(CH₂)₀₋₅-4- to 10-membered heterocyclyl comprising carbon atoms and 1-4 heteroatoms selected from N, NR₅ₐ, O, and S(═O)ₚ, and substituted with 1-4 R₅;
R₅, at each occurrence, is independently selected from the group consisting of H, F, Cl, Br, CN, OR_{b}, ═O, —(CH₂)₀₋₂—NR₁₀R₁₀, C₁₋₅ alkyl substituted with 1-3 Rₑ, C₃₋₆ carbocyclyl with 1-3 Rₑ, and 3- to 6-membered heterocyclyl comprising carbon atoms and 1-4 heteroatoms selected from N, NR_{5b}, O, and S(═O)ₚ, and substituted with 1-4 Rₑ;
R₅ₐ, at each occurrence, is independently selected from the group consisting of H, C₁₋₅ alkyl substituted with 1-4 Rₑ, C(═O)R_{b}, C(═O)OR_{b}, C₃₋₆carbocyclyl substituted with 1-3 Rₑ, and 5- to 8-membered heterocyclyl comprising carbon atoms and 1-4 heteroatoms selected from the group consisting of N, NR_{5b}, O, and S(═O)ₚ, and substituted with 1-3 Rₑ;
R_{5b}, at each occurrence, is independently selected from the group consisting of H and C₁₋₃ alkyl substituted with 1-3 Rₑ;
R₆, at each occurrence, is independently selected from the group consisting of H, F, Cl, Br, CN, —NRₐRₐ, —OR_{b}, —C(═O)NRₐRₐ, —C(═O)NRₐOR_{b}, —C(═O)R_{b}, —S(═O)₂Rₑ, —NRS(═O)₂Rₑ, C₁₋₄ alkyl substituted with 1-5 Rₑ, —(CH₂)C₃₋₆carbocyclyl substituted with 1-5 R₇, and 5- to 6-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-4 Rₑ, C₂₋₄ alkenyl substituted with 1-5 Rₑ, C₂₋₄ alkynyl substituted with 1-4 Rₑ, —(CH₂)ᵣC₃₋₆ carbocyclyl substituted with 1-4 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-4 R₇;
R₇, at each occurrence, is independently selected from the group consisting of H, F, Cl, Br, CN, OR_{b}, ═O, and C₁₋₃ alkyl;
R₇ₐ, at each occurrence, is independently selected from the group consisting of H and C₁₋₃ alkyl;
R₁₀, at each occurrence, is independently selected from the group consisting of H, C₁₋₄ alkyl substituted with 1-3 Rₑ, —(CH₂)ᵣ—C₃₋₁₀carbocyclyl substituted with 1-3 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H and C₁₋₃ alkyl;
R_{b}, at each occurrence, is independently selected from the group consisting of H, C₁₋₅ alkyl substituted with 1-5 Rₑ, C₃₋₁₀ carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-3 Rₑ;
R_{c}, at each occurrence, is independently selected from the group consisting of C₁₋₃ alkyl and C₃₋₆ cycloalkyl;
Rₑ, at each occurrence, is independently selected from the group consisting of H, F, Cl, Br, CN, ═O, C₁₋₃ alkyl substituted with 1-4 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, Br, OH, and CN.

5. The compound of claim 4, or a pharmaceutically acceptable salt thereof, wherein:
R₂ is selected from the group consisting 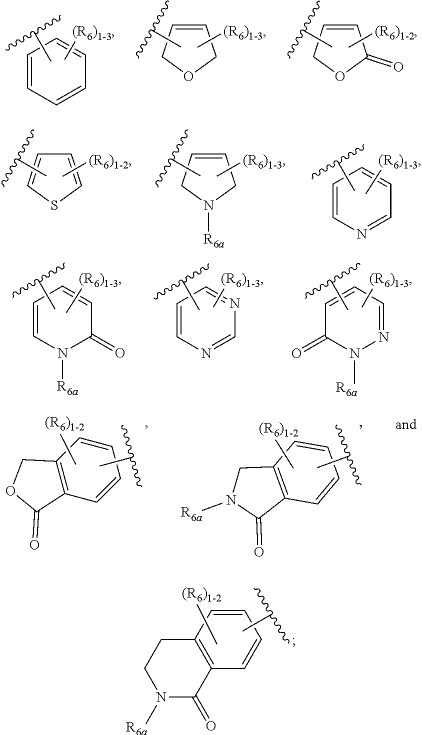
R₄′ is selected from the group consisting of F, Cl, CN, and C₁₋₃ alkyl;
R₄″ is selected from the group consisting of —O(CH₂)₁₋₃OR_{b}, —O(CH₂)₁₋₃C(═O)NRₐRₐ, —O(CH₂)₁₋₃NRₐC(═O)R_{b}, —O(CH₂)₁₋₃NRₐC(═O)R_{b}, —O(CH₂)₁₋₃NRₐRₐ, 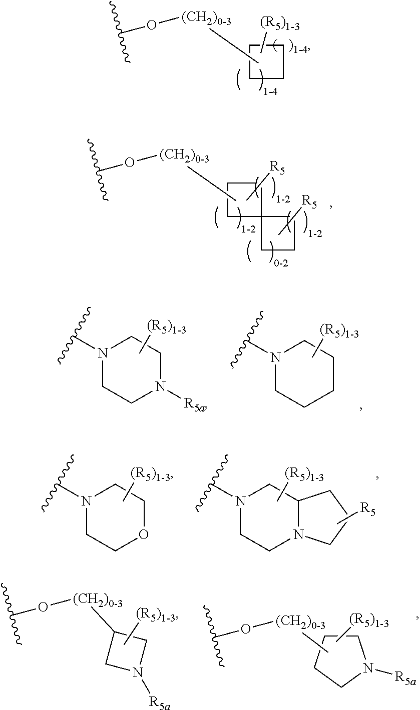 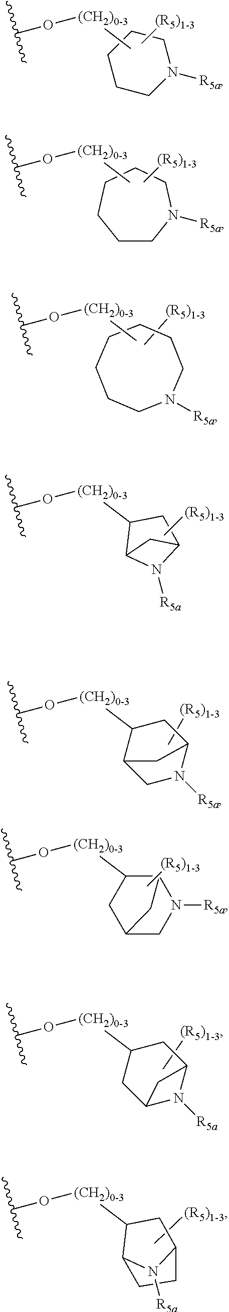 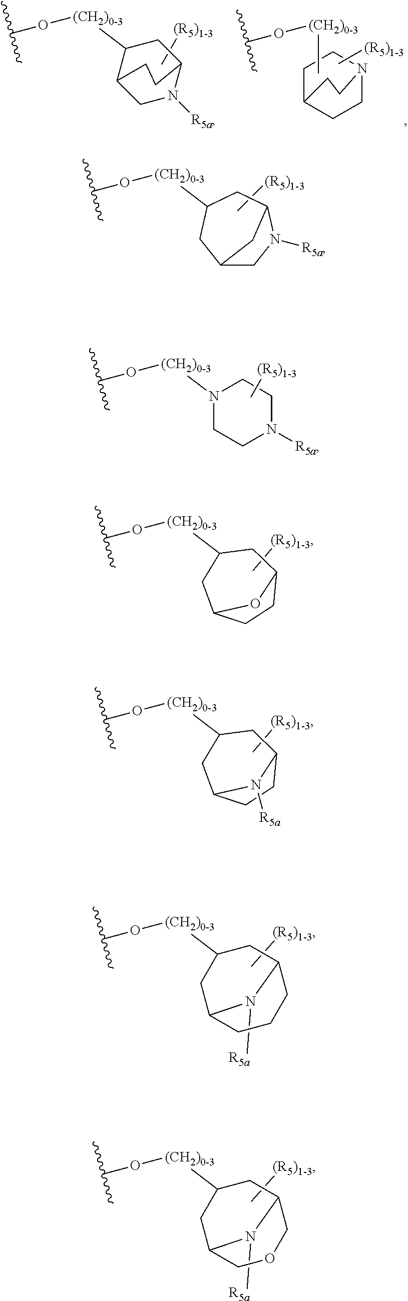 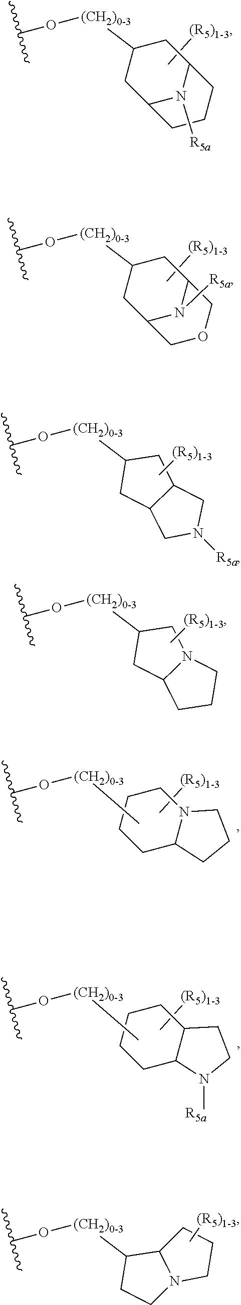 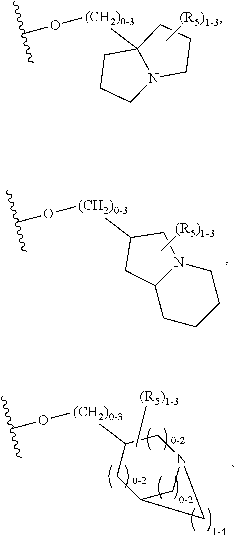 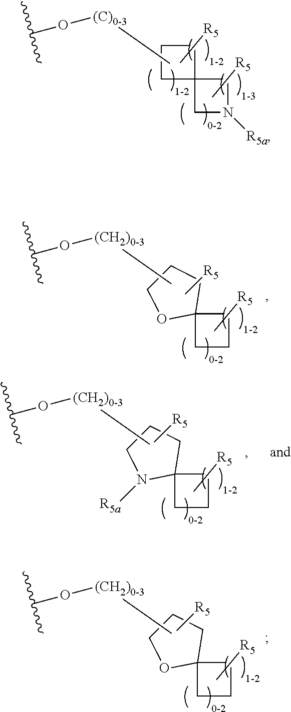
R₅, at each occurrence, is independently selected from the group consisting of H, F, Cl, Br, CN, OR_{b}, ═O, —(CH₂)₀₋₁—NR₁₀R₁₀, C₁₋₃ alkyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₄ alkyl substituted with 1-5 Rₑ;
R₆, at each occurrence, is independently selected from the group consisting of H, F, Cl, Br, CN, OR_{b}, C(═O)R_{b}, —C(═O)NRₐRₐ, —S(═O)₂Rₑ, C₁₋₃ alkyl substituted with 1-5 Rₑ, 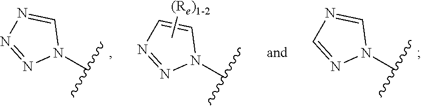
R₆ₐ, at each occurrence, is independently selected from the group consisting of —C(═O)R_{b}, —C(═O)OR_{b}, —S(═O)ₚR_{c}, C₁₋₃ alkyl substituted with 1-3 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₁₀, at each occurrence, is independently selected from the group consisting of H, C₁₋₅ alkyl substituted with 1-3 Rₑ, —(CH₂)ᵣ—C₃₋₆ carbocyclyl substituted with 1-3 Rₑ, and —(CH₂)ᵣ-3- to 6-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H, C(═O)OR_{b}, and C₁₋₃ alkyl;
R_{b}, at each occurrence, is independently selected from the group consisting of H, C₁₋₃ alky substituted with 1-4 Rₑ, C₃₋₆ carbocyclyl substituted with 1-3 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
R_{c} is C₁₋₃ alky substituted with 1-3 Rₑ;
Rₑ, at each occurrence, is independently selected from the group consisting of H, F, Cl, CN, C₁₋₃ alkyl substituted with 14 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, Br, OH, and CN.

6. The compound of claim 5, or a pharmaceutically acceptable salt thereof, wherein:
R₂ is selected from the group consisting 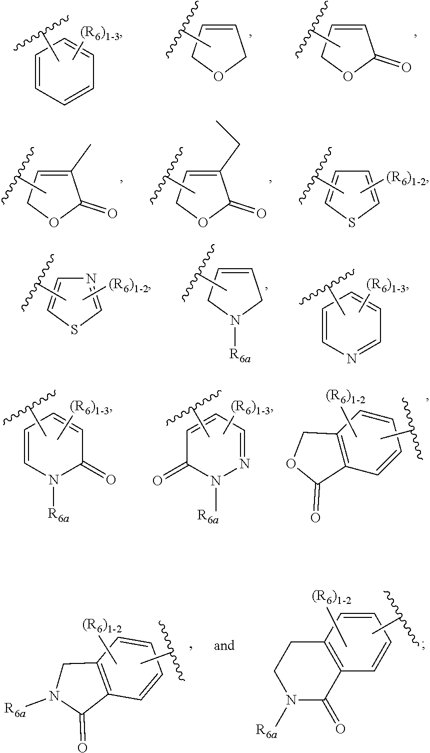
R₄′ is selected from the group consisting of F, Cl, CN, and C₁₋₃ alkyl;
R₄″ is selected from the group consisting of 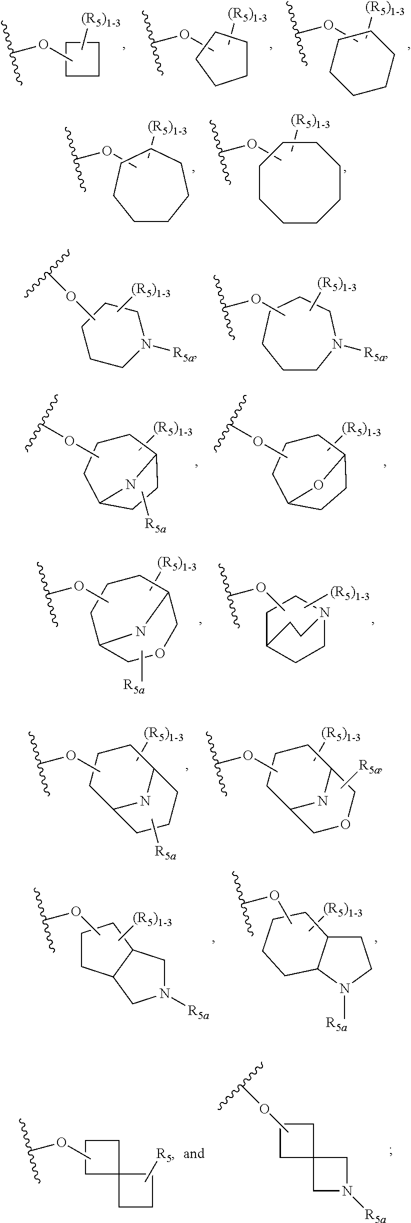
R₅, at each occurrence, is independently selected from the group consisting of H, F, Cl, Br, CN, OR_{b}, —NR₁₀R₁₀, C₁₋₃ alkyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₃ alkyl substituted with 1-4 Rₑ, and C(═O)R_{b};
R₆, at each occurrence, is independently selected from the group consisting of H, F, Cl, Br, CN, —C(═O)NRₐRₐ, —S(═O)₂Rₑ, C₁₋₃ alkyl substituted with 1-4 Rₑ, 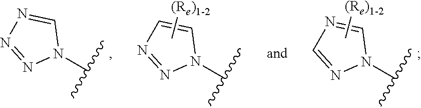
R₆ₐ, at each occurrence, is independently selected from the group consisting of —C(═O)R_{b}, —S(═O)ₚR_{c}, and C₁₋₃ alkyl substituted with 1-3 Rₑ;
R₁₀, at each occurrence, is independently selected from the group consisting of H, C₁₋₅ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H and C₁₋₃ alkyl;
R_{b}, at each occurrence, is independently selected from the group consisting of H, C₁₋₃ alky substituted with 1-3 Rₑ, and 5- to 6-membered heterocyclyl comprising carbon atoms and 1-4 heteroatoms selected from the group consisting of N, NR_{f}, O, and S(═O)ₚ, and substituted with 1-5 Rₑ;
R_{c} is C₁₋₃ alky;
Rₑ, at each occurrence, is independently selected from the group consisting of H, F, Cl, CN, C₁₋₃ alkyl substituted with 1-3 R_{g}, NR_{f}R_{f}, and —OR_{f};
R_{f} is independently selected from the group consisting of H and C₁₋₃ alkyl; and
R_{g} is independently selected from the group consisting of H, F, Cl, Br, OH, and CN.

7. The compound of claim 6, or a pharmaceutically acceptable salt thereof, wherein:
R₄″ is selected from the group consisting of 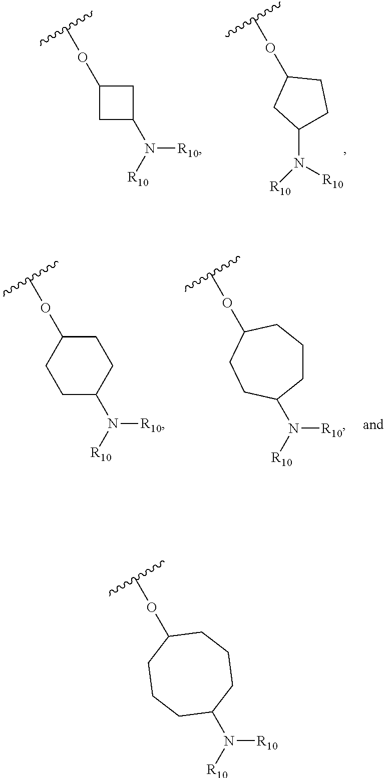
R₁₀, at each occurrence, is independently selected from the group consisting of H, C₁₋₅ alkyl substituted with 1-3 Rₑ, —(CH₂)₀₋₁—C₃₋₆ cycloalkyl substituted with 1-3 Rₑ, and 3- to 6-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a —(CH₂)₀₋₁3- to 6-membered heterocyclyl comprising carbon atoms and 1-4 heteroatoms selected from the group consisting of N, NR_{f}, and O, and substituted with 1-3 Rₑ;
Rₑ, at each occurrence, is independently selected from the group consisting of H, F, Cl, CN, C₁₋₃ alkyl substituted with 1-3 R_{g}, and —OR_{f};
R_{f} is selected from the group consisting of H and C₁₋₃ alkyl; and
R_{g}, at each occurrence, is independently selected from the group consisting of H, F, Cl, —OH, and CN.

8. The compound of claim 7, or a pharmaceutically acceptable salt thereof, wherein:
R₁₀, at each occurrence, is independently selected from the group consisting of H, CH₃, CD₃, CH₂CH₃, 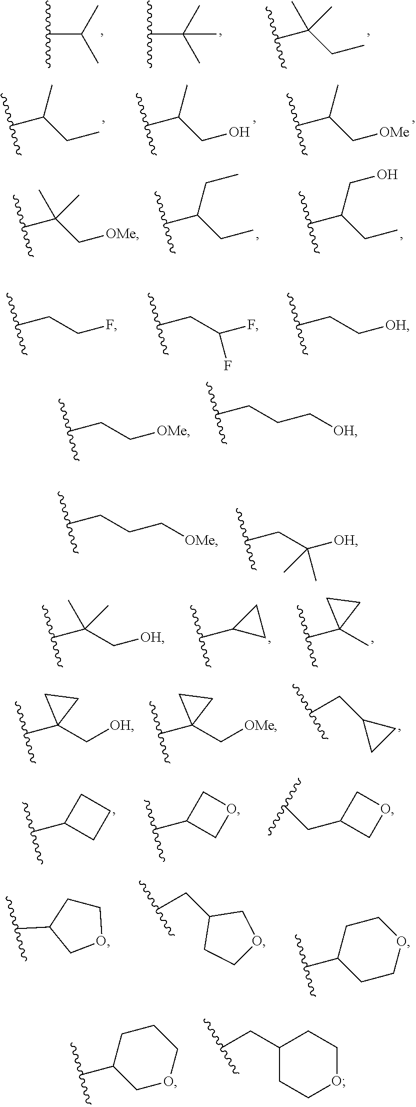
or R₁₀ and R₁₀ together with the nitrogen atom to which they are both attached form a heterocycyl selected from the group consisting of

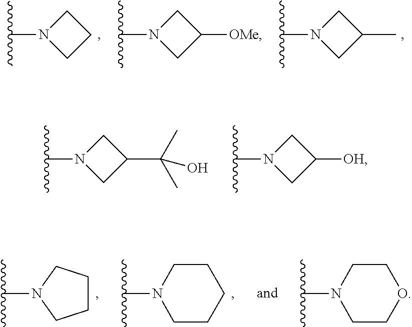

9. The compound of claim 2, or a pharmaceutically acceptable salt thereof, wherein:
R₁ is selected from the group consisting of 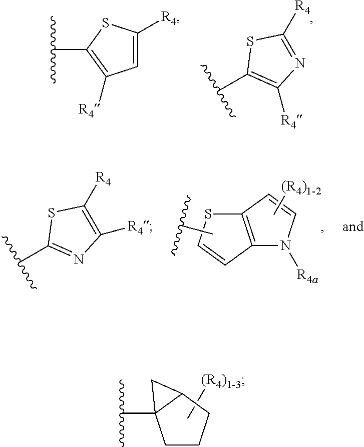
R₂ is selected from the group consisting of 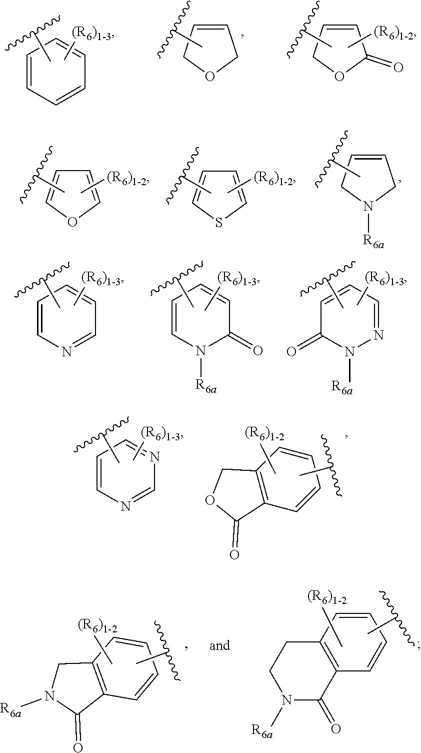
R₄, at each occurrence, is independently selected from the group consisting of H, F, Cl, Br, ═O and CN;
R₄ₐ, at each occurrence, is independently selected from the group consisting of H, —C(═O)NRₐRₐ, —C(═O)R_{b}, —S(═O)ₚR_{c}, —S(═O)ₚNRₐRₐ, and C₁₋₅ alkyl substituted with 1-5 Rₑ;
R₄′ is selected from the group consisting of F, Cl, CN, and C₁₋₃ alkyl substituted with 1-3 Rₑ;
R₄″ is selected from the group consisting of —OC₁₋₄ alkyl substituted with 1-2 Rₑ, —O(CH₂)₁₋₃NRₐRₐ, 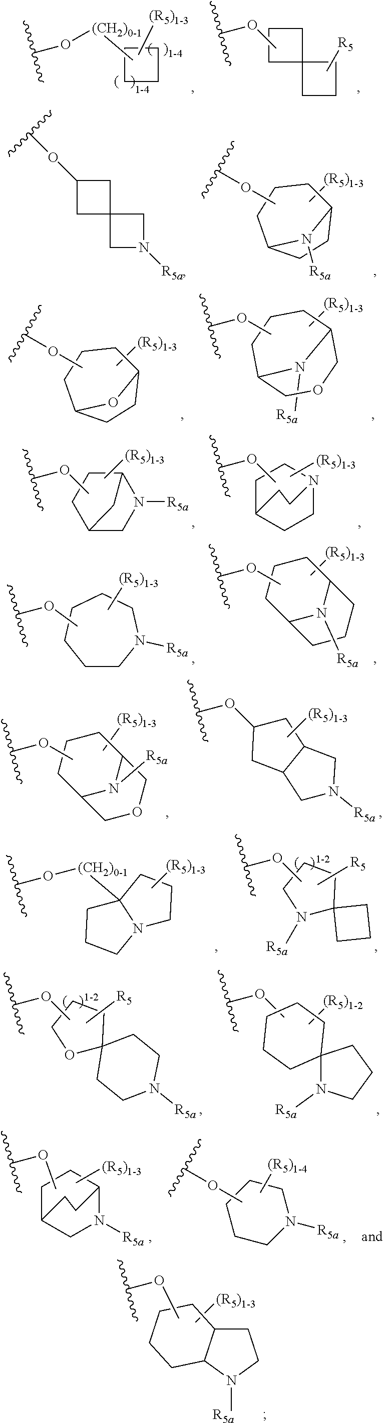
R₅, at each occurrence, is independently selected from the group consisting of H, F, Cl, Br, CN, OR_{b}, —(CH₂)₀₋₁—NR₁₀R₁₀, C₁₋₃ alkyl substituted with 1-5 Rₑ, C₃₋₆ carbocyclyl with 1-5 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H, C₁₋₃ alkyl substituted with 1-4 Rₑ, C(═O)R_{b}, and C₃₋₆ cycloalkyl;
R₆, at each occurrence, is independently selected from the group consisting of H, F, Cl, Br, CN, OR_{b}, —S(═O)₂Rₑ, C₁₋₃ alkyl substituted with 1-5 Rₑ, 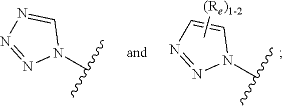
R₆ₐ, at each occurrence, is independently selected from the group consisting of —C(═O)R_{b}, —S(═O)ₚR_{c}, and C₁₋₃ alkyl substituted with 1-3 Rₑ;
R₁₀, at each occurrence, is independently selected from the group consisting of H, C₁₋₅ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, and O, and substituted with 1-3 Rₑ;
Rₐ, at each occurrence, is independently selected from the group consisting of H, C₁₋₄ alkyl substituted with 1-2 Rₑ, C₃₋₆ carbocyclyl, and 3- to 6-membered heterocyclyl comprising carbon atoms and 1-4 heteroatoms selected from the group consisting of N, NR_{f}, O, and S(═O)ₚ;
R_{b}, at each occurrence, is independently selected from the group consisting of H, C₁₋₃ alky substituted with 1-3 Rₑ, C₃₋₆ carbocyclyl substituted with 1-2 Rₑ; and 5- to 6-membered heterocyclyl comprising carbon atoms and 1-4 heteroatoms selected from the group consisting of N, NR_{f}, O, and S(═O)ₚ, and substituted with 1-2 Rₑ;
R_{c} is C₁₋₃ alky;
Rₑ, at each occurrence, is independently selected from the group consisting of H, F, Cl, CN, C₁₋₃ alkyl substituted with 1-3 R_{g}, NR_{f}R_{f}, and —OR_{f};
R_{f}, at each occurrence, is independently selected from the group consisting of H and C₁₋₃ alkyl; and
R_{g}, at each occurrence, is independently selected from the group consisting of H, F, Cl, OH, and CN.

10. The compound of claim 2, having Formula (III): 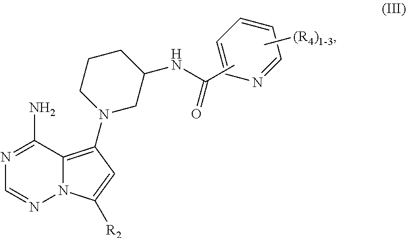
or a pharmaceutically acceptable salt thereof, wherein:
R₂ is selected from the group consisting of 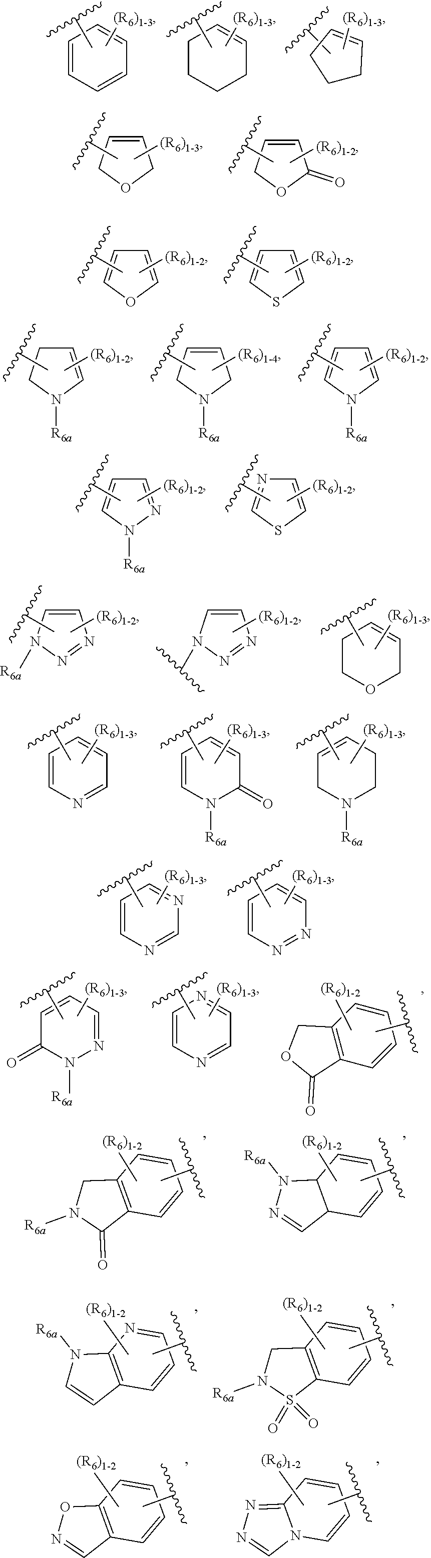 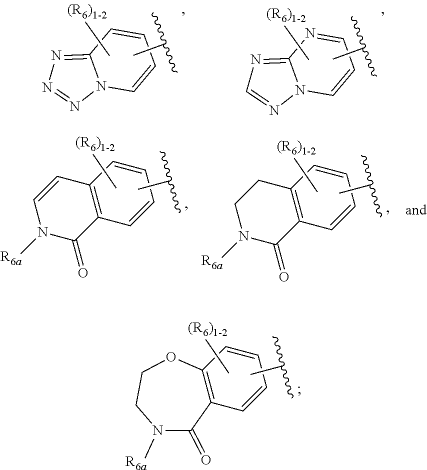
R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₁₀ carbocyclyl substituted with 1-5 R₅, —(CH₂)ᵣ—O—(CH₂)—C₃₋₁₀ carbocyclyl, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₅, at each occurrence, is independently selected from the group consisting of H, F, Cl, Br, CN, OR_{b}, ═O, —(CH₂)ᵣ—NR₁₀R₁₀, C₁₋₅ alkyl substituted with 1-4 Rₑ, C₃₋₆ carbocyclyl with 1-4 Rₑ, and 3- to 6-membered heterocyclyl comprising carbon atoms and 1-4 heteroatoms selected from N, NR_{5b}, O, and S(═O)ₚ, and substituted with 1-5 Rₑ;
R₅ₐ, at each occurrence, is independently selected from the group consisting of H and C₁₋₅ alkyl substituted with 1-4 Rₑ, C(═O)R_{b}, C(═O)OR_{b}, C₃₋₆carbocyclyl substituted with 1-4 Rₑ, and heterocyclyl comprising carbon atoms and 1-4 heteroatoms selected from the group consisting of N, NR_{5b}, O, and S(═O)ₚ, and substituted with 1-4 Rₑ;
R_{5b}, at each occurrence, is independently selected from the group consisting of H and C₁₋₄ alkyl substituted with 1-4 Rₑ;
R₆, at each occurrence, is independently selected from the group consisting of H, F, Cl, Br, CN, ═O, —(CH₂)ᵣNRₐRₐ, NO₂, —OR_{b}, —C(═O)NRₐRₐ, —C(═O)NRₐ(CH₂)ᵣOR_{b}, —C(═O)R_{b}, —C(═O)(CH₂)ᵣOR_{b}, —NRₐC(═O)R_{b}, —NRₐC(═O)OR_{b}, —NRₐC(═O)(CH₂)ᵣNRₐRₐ, —S(═O)₂Rₑ, —NRₐS(═O)₂Rₑ, C₁₋₄ alkyl substituted with 1-5 Rₑ, C₂₋₄ alkenyl substituted with 1-5 Rₑ, —(CH₂)ᵣ—C₃₋₆carbocyclyl substituted with 1-5 R₇, and —(CH₂)ᵣ-5- to 8-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-5 Rₑ, C₂₋₄ alkenyl substituted with 1-5 Rₑ, C₂₋₆ alkynyl substituted with 1-5 Rₑ, —(CH₂)ᵣC₃₋₆ carbocyclyl substituted with 1-5 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-5 R₇;
R₇, at each occurrence, is independently selected from the group consisting of H, F, Cl, Br, CN, OR_{b}, ═O, C₁₋₄ alkyl substituted with 1-4 Rₑ, C₂₋₄ alkenyl substituted with 1-4 Rₑ, and C₂₋₄ alkynyl substituted with 1-4 Rₑ;
R₇ₐ, at each occurrence, is independently selected from the group consisting of H and C₁₋₄ alkyl substituted with 1-5 Rₑ;
R₁₀, at each occurrence, is independently selected from the group consisting of H, C₁₋₅ alkyl substituted with 1-4 Rₑ, —(CH₂)ᵣ—C₃₋₁₀carbocyclyl substituted with 1-4 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-4 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-4 Rₑ;
Rₐ, at each occurrence, is independently selected from the group consisting of H, C₁₋₅ alkyl substituted with 1-4 Rₑ, C₃₋₁₀ carbocyclyl substituted with 1-4 Rₑ, and —(CH₂)ᵣ-heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 1-4 Rₑ, C₂₋₅ alkynyl substituted with 1-4 Rₑ, C₃₋₁₀ carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c}, at each occurrence, is independently selected from the group consisting of C₁₋₅ alkyl substituted with 1-4 Rₑ, C₃₋₆carbocyclyl substituted with 1-4 Rₑ, and heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H, C₁₋₄ alkyl, and C₃₋₆ cycloalkyl;
Rₑ, at each occurrence, is independently selected from the group consisting of H, F, Cl, Br, CN, ═O, C₁₋₅ alkyl substituted with 1-4 R_{g}, C₂₋₅ alkenyl substituted with 1-4 R_{g}, C₂₋₅ alkynyl substituted with 1-4 R_{g}, —(CH₂)ᵣC₃₋₆ cycloalkyl, —(CH₂)ᵣ-heterocyclyl comprising carbon atoms and 1-4 heteroatoms selected from the group consisting of N, NR_{f}, O, and S(═O)_{P}, and substituted with 1-4 R_{g}, —OR_{f}, —(CH₂)ᵣS(═O)₂C₁₋₅ alkyl, —(CH₂)ᵣNR_{f}R_{f}, —(CH₂)C(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a heterocyclic ring;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4, or 5.

11. The compound of claim 2, or a pharmaceutically acceptable salt thereof, wherein:
R₁ is selected from the group consisting of 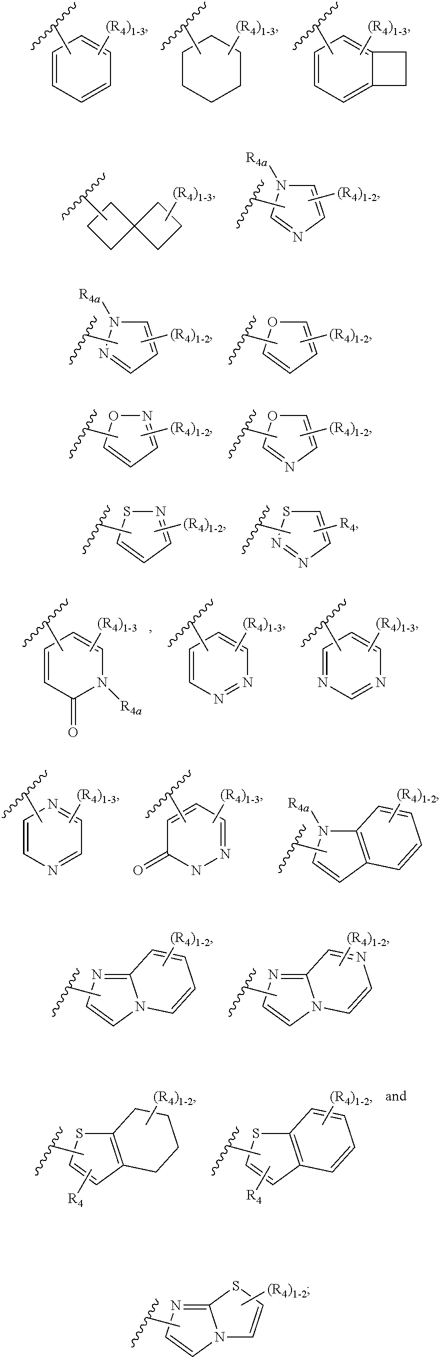
R₂ is selected from the group consisting of 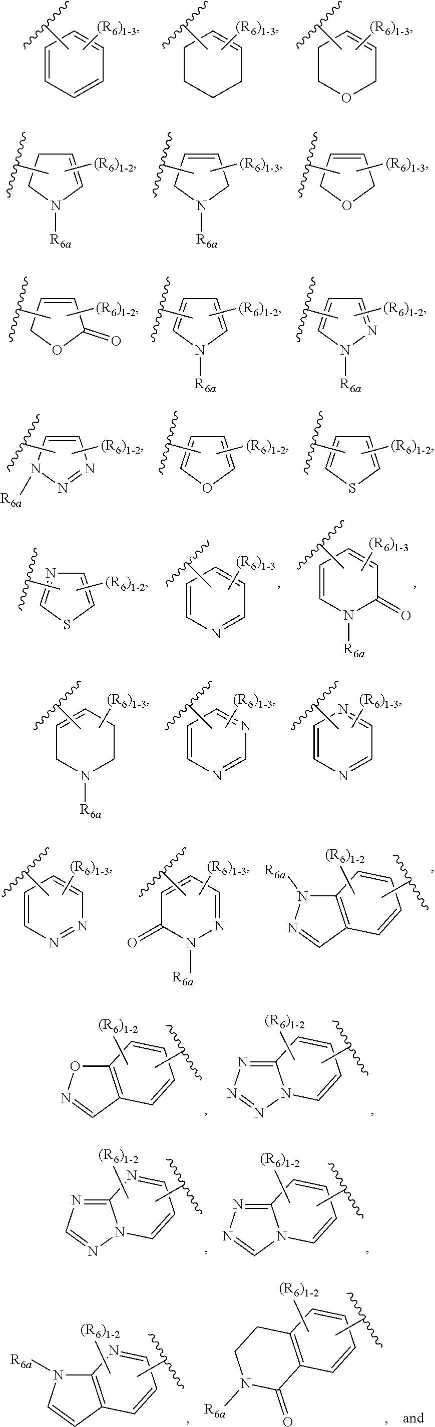 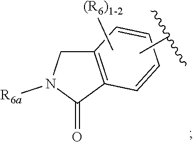
R₄, at each occurrence, is independently selected from the group consisting of H, F, Cl, Br, ═O, CN, —(CH₂)ᵣNRₐRₐ, —OR_{b}, —O(CH₂)₁₋₃OR_{b}, —O(CH₂)ᵣC(═O)NRₐRₐ, —O(CH₂)₁₋₃NRₐC(═O)R_{b}, —O(CH₂)₁₋₃NRₐC(═O)OR_{b}, —O(CH₂)₁₋₃NRₐRₐ, —C(═O)NRₐRₐ, —C(═O)R_{b}, —NRₐC(═O)OR_{b}, —NRₐC(═O)(CH₂)ᵣNRₐRₐ, C₁₋₄ alkyl substituted with 1-5 Rₑ, C₂₋₄ alkenyl substituted with 1-5 Rₑ, and C₂₋₄ alkynyl substituted with 1-5 Rₑ, —(CH₂)ᵣ—C₃₋₁₀ carbocyclyl substituted with 1-5 R₅, —(CH₂)ᵣ—O—(CH₂)—C₃₋₁₀ carbocyclyl, —(CH₂)ᵣ-4- to 10-membered heterocyclyl comprising carbon atoms and 1-4 heteroatoms selected from N, NR₅ₐ, O, and S(═O)ₚ, and substituted with 1-5 R₅, and —(CH₂)ᵣ—O—(CH₂)ᵣ-4- to 10-membered heterocyclyl comprising carbon atoms and 1-4 heteroatoms selected from N, NR₅ₐ, O, and S(═O)ₚ, and substituted with 1-5 R₅;
R₄ₐ, at each occurrence, is independently selected from the group consisting of H and C₁₋₄ alkyl substituted with 1-5 Rₑ;
R₅, at each occurrence, is independently selected from the group consisting of H, F, Cl, Br, CN, OR_{b}, ═O, —(CH₂)₀₋₂—NR₁₀R₁₀, C₁₋₅ alkyl substituted with 1-3 Rₑ, C₂₋₆ alkenyl substituted with 1-5 Rₑ, C₂₋₆ alkynyl substituted with 1-3 Rₑ, C₃₋₆ carbocyclyl with 1-3 Rₑ, and 3- to 6-membered heterocyclyl comprising carbon atoms and 1-4 heteroatoms selected from N, NR_{5b}, O, and S(═O)ₚ, and substituted with 1-4 Rₑ;
R₅ₐ, at each occurrence, is independently selected from the group consisting of H, C₁₋₃ alkyl substituted with 1-4 Rₑ, C(═O)R_{b}, C(═O)OR_{b}, C₃₋₆carbocyclyl substituted with 1-5 Rₑ, and 3- to 10-membered heterocyclyl comprising carbon atoms and 1-4 heteroatoms selected from the group consisting of N, NR_{5b}, O, and S(═O)ₚ, and substituted with 1-5 Rₑ;
R_{5b}, at each occurrence, is independently selected from the group consisting of H and C₁₋₆ alkyl substituted with 1-5 Rₑ;
R₆, at each occurrence, is independently selected from the group consisting of H, F, Cl, CN, OR_{b}, C(═O)R_{b}, —C(═O)NRₐRₐ, —S(═O)₂Rₑ, and 5- to 6-membered heterocyclyl comprising carbon atoms and 1-4 heteroatoms selected from the group consisting of N, NR₇ₐ, O, and S(═O)ₚ, and substituted with 1-5 R₇;
R₆ₐ, at each occurrence, is independently selected from the group consisting of H, —C(═O)NRₐRₐ, —C(═O)R_{b}, —C(═O)(CHR_{d})ᵣOR_{b}, —S(═O)ₚR_{c}, —S(═O)ₚNRₐRₐ, C₁₋₄ alkyl substituted with 1-4 Rₑ, C₂₋₄ alkenyl substituted with 1-5 Rₑ, C₂₋₄ alkynyl substituted with 1-4 Rₑ, —(CH₂)ᵣC₃₋₆ carbocyclyl substituted with 1-4 R₇, and —(CH₂)ᵣ-4- to 8-membered heterocyclyl comprising carbon atoms and 1-4 heteroatoms selected from the group consisting of N, NR₇ₐ, O, and S(═O)ₚ, and substituted with 1-4 R₇;
R₇, at each occurrence, is independently selected from the group consisting of H, F, Cl, Br, OR_{b}, ═O, CN, and C₁₋₃ alkyl;
R₇ₐ, at each occurrence, is independently selected from the group consisting of H and C₁₋₃ alkyl;
R₁₀, at each occurrence, is independently selected from the group consisting of H, C₁₋₄ alkyl substituted with 1-3 Rₑ, —(CH₂)ᵣ—C₃₋₁₀carbocyclyl substituted with 1-3 Rₑ, and —(CH₂)ᵣ-3- to 10-membered heterocyclyl comprising carbon atoms and 1-4 heteroatoms selected from the group consisting of N, NR_{f}, O, and S(═O)ₚ, and substituted with 1-3 Rₑ; or R₁₀ and R₁₀ together with the nitrogen atom to which they are both attached form a 3- to 10-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H, C(═O)OR_{b}, C₁₋₅ alkyl substituted with 1-4 Rₑ, C₃₋₁₀ carbocyclyl substituted with 1-4 Rₑ, and —(CH₂)ᵣ-heterocyclyl comprising carbon atoms and 1-4 heteroatoms selected from the group consisting of N, NR_{f}, O, and S(═O)ₚ, and substituted with 1-4 Rₑ; or Rₐ and Rₐ together with the nitrogen atom to which they are both attached form a heterocyclyl comprising carbon atoms and 1-4 heteroatoms selected from the group consisting of N, NR_{f}, O, and S(═O)ₚ, and substituted with 1-4 Rₑ;
R_{b}, at each occurrence, is independently selected from the group consisting of H, C₁₋₅ alkyl substituted with 1-5 Rₑ, C₂₋₅ alkenyl substituted with 1-4 Rₑ, C₂₋₅ alkynyl substituted with 1-4 Rₑ, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c}, at each occurrence, is independently selected from the group consisting of C₁₋₅ alkyl substituted with 1-4 Rₑ, C₃₋₆carbocyclyl substituted with 1-4 Rₑ, and heterocyclyl comprising carbon atoms and 1-4 heteroatoms selected from the group consisting of N, NR_{f}, O, and S(═O)ₚ, and substituted with 1-4 Rₑ;
R_{d}, at each occurrence, is independently selected from the group consisting of H, C₁₋₄ alkyl, and C₃₋₆ cycloalkyl;
Rₑ, at each occurrence, is independently selected from the group consisting of H, F, Cl, Br, CN, ═O, C₁₋₅ alkyl substituted with 1-4 R_{g}, C₂₋₅ alkenyl substituted with 1-4 R_{g}, C₂₋₅ alkynyl substituted with 14 R_{g}, —(CH₂)ᵣC₃₋₆ cycloalkyl, —(CH₂)ᵣ-heterocyclyl comprising carbon atoms and 1-4 heteroatoms selected from the group consisting of N, NR_{f}, O, and S(═O)_{P}, and substituted with 1-4 R_{g}, —OR_{f}, —(CH₂)ᵣS(═O)₂C₁₋₅ alkyl, —(CH₂)ᵣNR_{f}R_{f}, —(CH₂)ᵣC(═O)R_{f}, and —(CH₂)ᵣC(═O)OR_{f};
R_{f}, at each occurrence, is independently selected from the group consisting of H, C₁₋₅ alkyl substituted with 1-2 R_{g}, C₃₋₆ cycloalkyl; or R_{f} and R_{f} together with the nitrogen atom to which they are both attached form a heterocyclic ring;
R_{g}, at each occurrence, is independently selected from the group consisting of H, F, Cl, Br, OH, CN, NH₂, C₁₋₅ alkyl, and C₃₋₆ cycloalkyl;
p is an integer of zero, 1, or 2; and
r is an integer of zero, 1, 2, 3, 4, or 5.

12. The compound of claim 11, or a pharmaceutically acceptable salt thereof, wherein:
R₂ is selected from the group consisting of 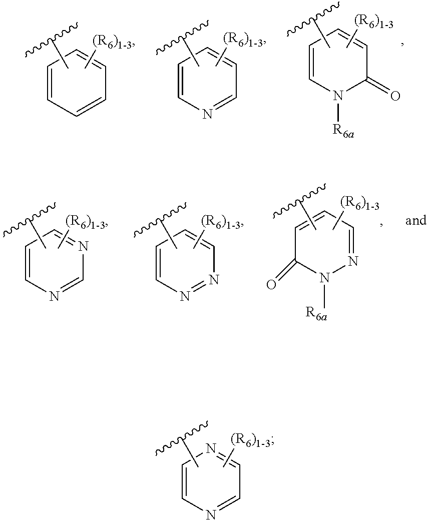
R₆, at each occurrence, is independently selected from the group consisting of H, F, Cl, CN, OR_{b}, C(═O)R_{b}, —C(═O)NRₐRₐ, —S(═O)₂Rₑ, C₁₋₃ alkyl substituted with 1-5 Rₑ, 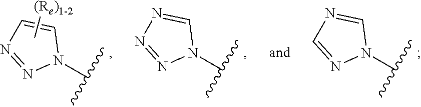
R₆ₐ, at each occurrence, is independently selected from the group consisting of H, C(═O)R_{b}, C(═O)OR_{b}, and C₁₋₃ alkyl substituted with 1-5 Rₑ;
Rₐ, at each occurrence, is independently selected from the group consisting of H, C₁₋₃ alkyl, and C(═O)OR_{b};
R_{b}, at each occurrence, is independently selected from the group consisting of H, C₁₋₃ alkyl substituted with 1-5 Rₑ, C₃₋₁₀ carbocyclyl substituted with 1-5 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c} is C₁₋₃ alkyl;
Rₑ, at each occurrence, is independently selected from the group consisting of H, F, Cl, CN, and OR_{f}; and
R_{f}, at each occurrence, is independently selected from the group consisting of H and C₁₋₃ alkyl.

13. The compound of claim 11, or a pharmaceutically acceptable salt thereof, wherein:
R₂ is selected from the group consisting of 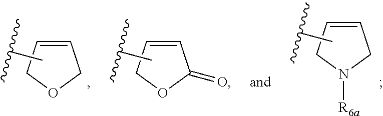
R₆ₐ, is selected from the group consisting of H, C(═O)R_{b}, C(═O)OR_{b}, —S(═O)₂Rₑ, and C₁₋₃ alkyl substituted with 1-5 Rₑ;
R_{b}, at each occurrence, is independently selected from the group consisting of H, C₁₋₃ alkyl, C₃₋₁₀carbocyclyl substituted with 1-4 Rₑ, and 5- to 6-membered heterocyclyl comprising carbon atoms and 1-4 heteroatoms selected from the group consisting of N, NR_{f}, O, and S(═O)ₚ, and substituted with 1-4 Rₑ;
R_{c} is C₁₋₃ alkyl;
Rₑ, at each occurrence, is independently selected from the group consisting of H, F, Cl, CN, and OR_{f}; and
R_{f}, at each occurrence, is independently selected from the group consisting of H and C₁₋₃ alkyl.

14. The compound according to claim 11, or a pharmaceutically acceptable salt thereof, wherein;
R₁ is selected from the group consisting of 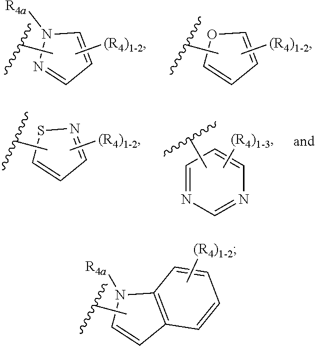
R₁ is selected from the group consisting of 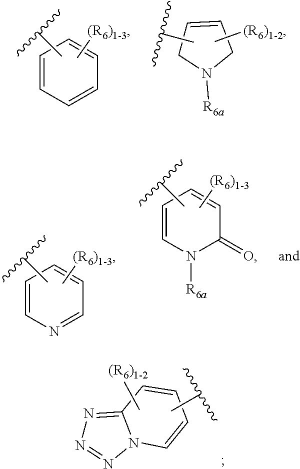
R₄, at each occurrence, is independently selected from the group consisting of H, F, Cl, —OR_{b}, —O(CH₂)₁₋₄NRₐRₐ, C₁₋₃ alkyl substituted with 1-5 Rₑ, C₃₋₆ carbocyclyl substituted with 1-5 R₅, —O—(CH₂)ᵣC₃₋₆ carbocyclyl substituted with 1-5 R₅, 4- to 10-membered heterocyclyl substituted with 1-5 R₅, and —O—(CH₂)ᵣ-4- to 10-membered heterocyclyl substituted with 1-5 R₅, wherein the C₃₋₆ carbocyclyl is selected from the group consisting of C₃₋₆ cycloalkyl and phenyl and the heterocyclyl is selected from the group consisting of 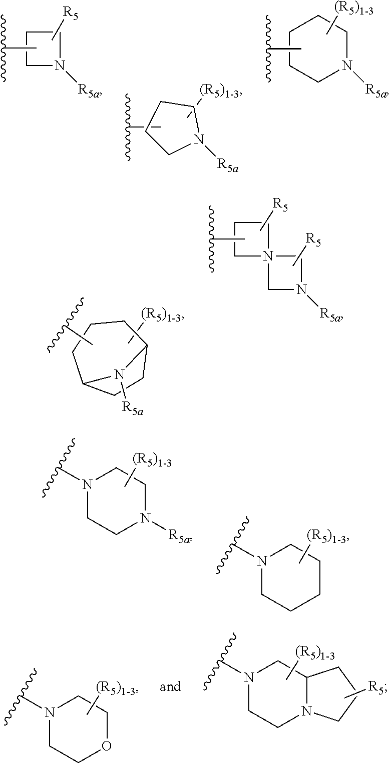
R₄ₐ, at each occurrence, is independently selected from the group consisting of H and C₁₋₃ alkyl;
R₅, at each occurrence, is independently selected from the group consisting of H, F, Cl, Br, CN, OH, ═O, —NR₁₀R₁₀, C₁₋₃ alkyl substituted with 1-5 Rₑ, —OC₁₋₅ alkyl substituted with 1-5 Rₑ, and C₃₋₆ cycloalkyl substituted with 1-5 Rₑ;
R₅ₐ, at each occurrence, is independently selected from the group consisting of H, C₁₋₃ alkyl substituted with 1-4 Rₑ, C(═O)R_{b}, and C(═O)OR_{b};
R_{5b}, at each occurrence, is independently selected from the group consisting of H and C₁₋₆ alkyl substituted with 1-5 Rₑ;
R₆, at each occurrence, is independently selected from the group consisting of H, F, Cl, Br, CN, OR_{b}, C(═O)R_{b}, —C(═O)NRₐRₐ, —S(═O)₂Rₑ, C₁₋₃ alkyl substituted with 1-5 Rₑ, 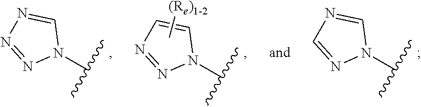
R₆ₐ, at each occurrence, is independently selected from the group consisting of H, C(═O)R_{b}, C(═O)OR_{b}, and C₁₋₃ alkyl substituted with 1-5 Rₑ;
R₁₀, at each occurrence, is independently selected from the group consisting of H and C₁₋₄ alkyl substituted with 1-3 Rₑ; or R₁₀ and R₁₀ together with the nitrogen atom to which they are both attached form a 3- to 6-membered heterocyclyl comprising carbon atoms and 1-4 heteroatoms selected from the group consisting of N, NR_{f}, O, and S(═O)ₚ, and substituted with 1-3 Rₑ;
Rₐ, at each occurrence, is independently selected from the group consisting of H, C₁₋₃ alkyl, and C(═O)OR_{b};
R_{b}, at each occurrence, is independently selected from the group consisting of H, C₁₋₃ alkyl, and 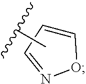
R_{c}, at each occurrence, is C₁₋₃ alkyl substituted with 1-5 Rₑ;
Rₑ, at each occurrence, is independently selected from the group consisting of H, F, Cl, CN, and OR_{f};
R_{f}, at each occurrence, is independently selected from the group consisting of H and C₁₋₃ alkyl; and
r is an integer of zero or 1.

15. The compound of claim 1, having Formula (VII): 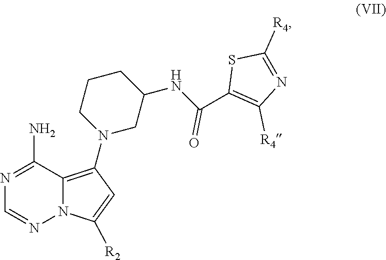
or a pharmaceutically acceptable salt thereof, wherein:
R₂ is selected from the group consisting of CN, 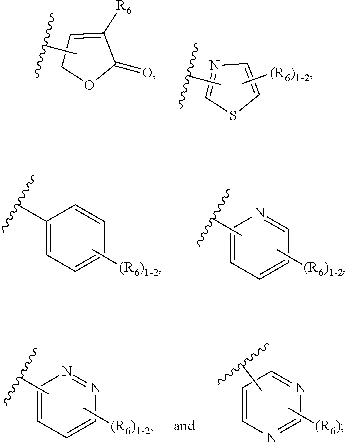
R₄′ is selected from the group consisting of CH₃ and CD₃;
R₄″ is selected from the group consisting of 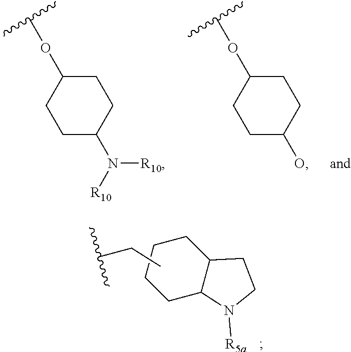
R₅ₐ is C₁₋₃ alkyl;
R₆, at each occurrence, is independently selected from the group consisting of H, OR_{b}, —C(═O)OR_{b}, —C(═O)NRₐRₐ, —S(═O)₂Rₑ, and C₁₋₃ alkyl,
R₁₀, at each occurrence, is independently selected from the group consisting of H, CH₃, CD₃, CH₂CH₃, 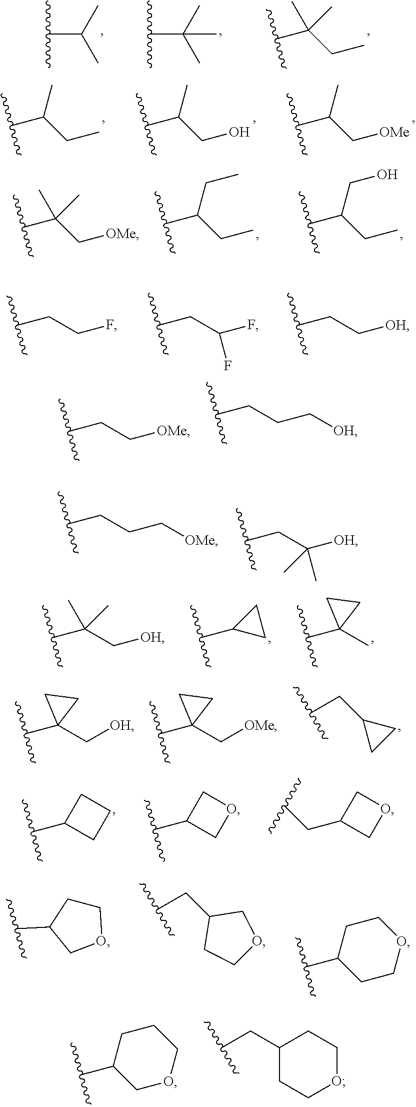
or R₁₀ and R₁₀ together with the nitrogen atom to which they are both attached form a heterocyclyl selected from the group consisting of 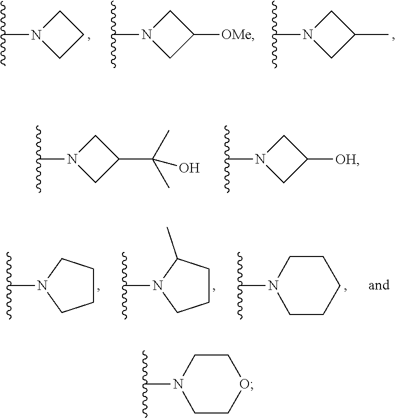
and
R_{b} is selected from the group consisting CH₃ and CD₃.

16. The compound of claim 15, having Formula (X): 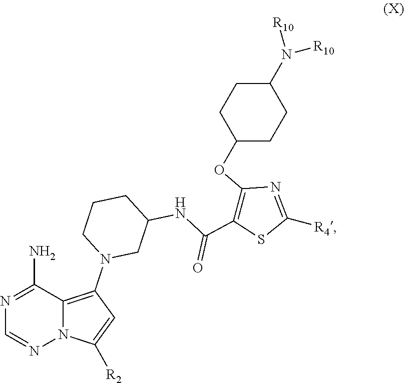
or a pharmaceutically acceptable salt thereof, wherein:
R₂ is selected from the group consisting of 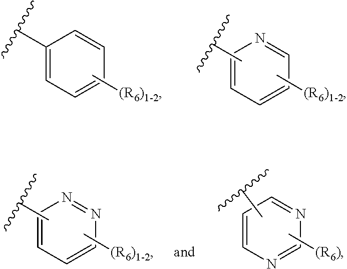
R₄′ is selected from the group consisting of CH₃ and CD₃;
R₆, at each occurrence, is independently selected from the group consisting of OR_{b}, C(═O)R_{b}, —C(═O)NRₐRₐ, —S(═O)₂Rₑ, C₁₋₃ alkyl substituted with 1-5 Rₑ,
R₁₀, at each occurrence, is independently selected from the group consisting of H, CH₃, CD₃, CH₂CH₃, 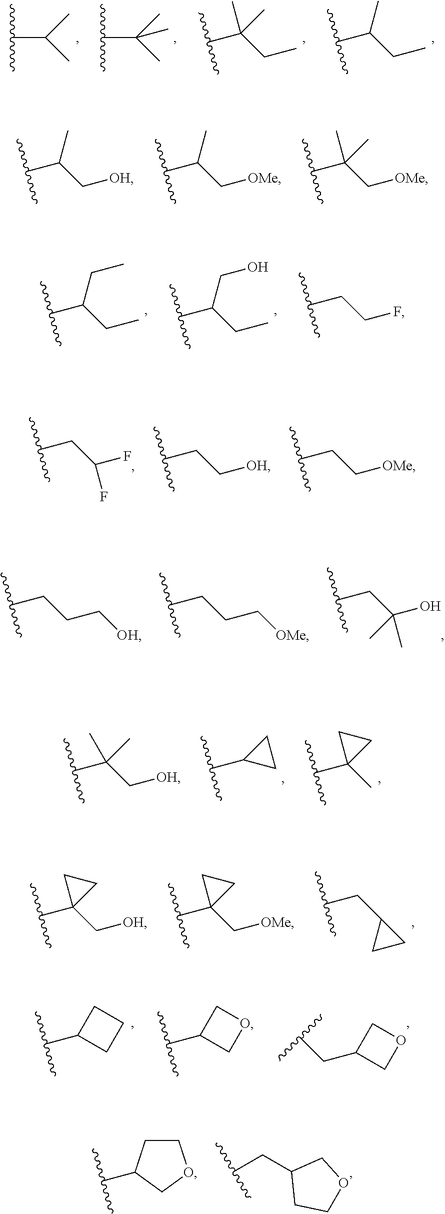 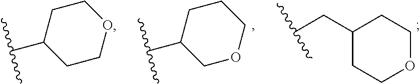 [SMILES: *C1CCOCC1 ; *C1CCCOC1 ; *CC1CCOCC1]
or R₁₀ and R₁₀ together with the nitrogen atom to which they are both attached form a heterocyclyl selected from the group consisting of

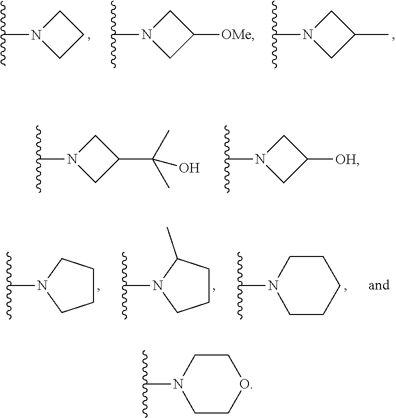

17. The compound of claim 16, or a pharmaceutically acceptable salt thereof, wherein:
R₂ is selected from the group consisting of

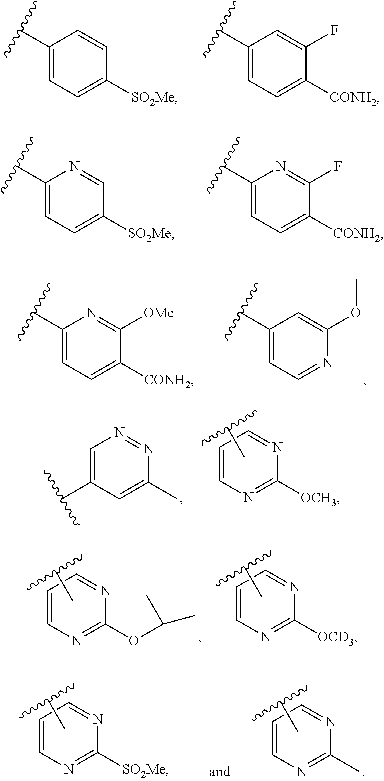

18. The compound of claim 15, having Formula (X): 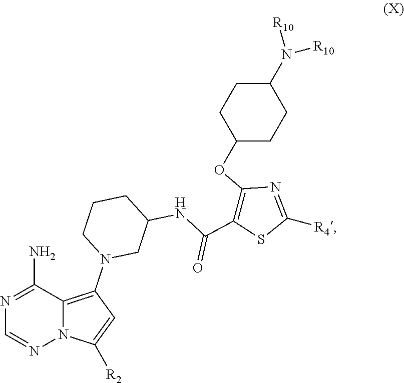
or a pharmaceutically acceptable salt thereof, wherein:
R₂ is selected from the group consisting of 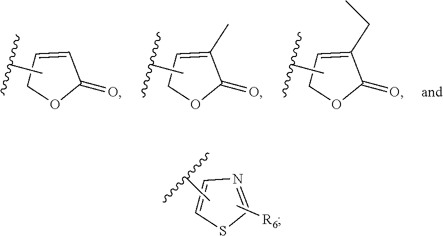
R₄′ is selected from the group consisting of CH₃ and CD₃;
R₆ is selected from the group consisting of —C(═O)NH₂, —C(═O)NHCH₃, and —C(═O)OH;
R₁₀, at each occurrence, is independently selected from the group consisting of H, CH₃, CD₃, CH₂CH₃, 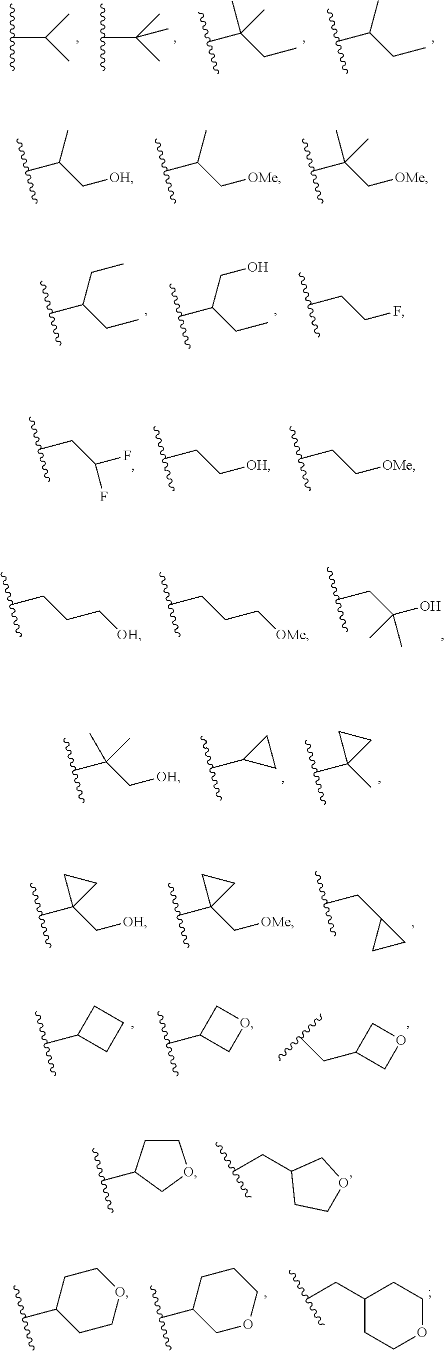
or R₁₀ and R₁₀ together with the nitrogen atom to which they are both attached form a heterocyclyl selected from the group consisting of

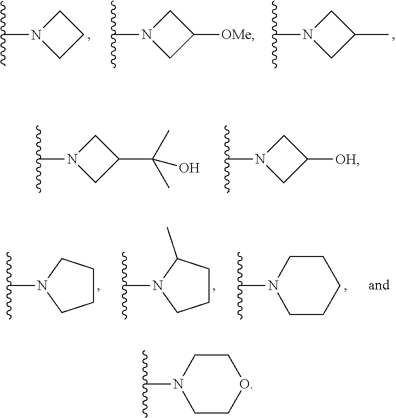

19. The compound of claim 1, which is selected from any one of the examples as described in the specification, or a pharmaceutically acceptable salt thereof.

20. A pharmaceutical composition comprising one or more compounds according to claim 1 and a pharmaceutically acceptable carrier or diluent.

21. (canceled)

22. A method for prophylaxis and/or treatment of a disorder associated with serum- and glucocorticoid-regulated kinase 1 (SGK1) activity, comprising administering to a patient in need thereof a therapeutically effective amount of the pharmaceutical composition of claim 20.

23. The method of claim 22, wherein said disorder is selected from cardiovascular and cerebrovascular diseases (including hypertension, heart failure, coronary artery disease, myocardial infarction, peripheral vascular disease, stroke and arrhythmia), fibrotic diseases (including diabetic nephropathy, glomerulonephritis, experimental nephrotic syndrome, obstructive nephropathy, lung fibrosis, liver cirrhosis, fibrotic pancreatitis, peritoneal fibrosis, Crohn's disease and coeliac disease), metabolic disorders and complications (including diabetes, obesity, metabolic syndrome), immune/inflammatory diseases (including rheumatoid arthritis and osteoarthritis), neuronal diseases (including Alzheimer, Parkinson, seizure, and Lafora progressive myoclonic epilepsy) and cancer.
